(19) United States
(12) Patent Application Publication
(10) Pub. No.: US 2024/0023436 A1
(43) Pub. Date: Jan. 18, 2024

(54) ORGANIC ELECTROLUMINESCENT ELEMENT AND ELECTRONIC DEVICE

(71) Applicant: IDEMITSU KOSAN CO.,LTD., Tokyo (JP)

(72) Inventors: Satomi TASAKI, Tokyo (JP); Hiroaki ITOI, Tokyo (JP); Taro YAMAKI, Tokyo (JP); Maiko IIDA, Tokyo (JP); Kazuki NISHIMURA, Tokyo (JP); Yuki NAKANO, Tokyo (JP)

(73) Assignee: IDEMITSU KOSAN CO.,LTD., Tokyo (JP)

(21) Appl. No.: 17/918,728

(22) PCT Filed: Mar. 11, 2021
(86) PCT No.: PCT/JP2021/009835

§ 371 Date: Oct. 13, 2022

(30) Foreign Application Priority Data
Apr. 15, 2020 (JP) 2020-073089
Jun. 29, 2020 (JP) 2020-111889

(51) Int. Cl. H10K 85/60; C07D 307/91; C09K 11/06; C07D 307/77; C07C 15/38

(52) U.S. Cl. CPC H10K 85/6574; C07D 307/91; C09K 11/06; H10K 85/615; H10K 50/11; C07C 15/38; H10K 85/626; H10K 85/622; C07D 307/77

(57) ABSTRACT

There is provided an organic electroluminescence device including: an anode; a cathode; a first emitting layer provided between the anode and the cathode and containing a first compound; and a second emitting layer provided between the first emitting layer and the cathode and containing a second compound, in which at least one of the first emitting layer or the second emitting layer contains a compound having at least one deuterium atom, at least one of the first emitting layer or the second emitting layer contains a compound having a fused ring that includes four or more rings, and the first emitting layer and the second emitting layer are in direct contact with each other.

### TECHNICAL FIELD

The present invention relates to an organic electroluminescence device and an electronic device.

### BACKGROUND ART

An organic electroluminescence device (hereinafter, occasionally referred to as “organic EL device”) has found its application in a full-color display for mobile phones, televisions and the like. When a voltage is applied to an organic EL device, holes and electrons are injected from an anode and a cathode, respectively, into an emitting layer. The injected holes and electrons are recombined in the emitting layer to form excitons. Specifically, according to the electron spin statistics theory, singlet excitons and triplet excitons are generated at a ratio of 25%:75%.

Various studies have been made for compounds to be used for the organic EL device in order to enhance the performance of the organic EL device (see, for instance, Patent Literature 1). The performance of the organic EL device is evaluable in terms of, for instance, luminance, emission wavelength, chromaticity, luminous efficiency, drive voltage, and lifetime.

### CITATION LIST

### Patent Literature(s)

Patent Literature 1: JP 2019-161218 A

### SUMMARY OF THE INVENTION

### Problems to be Solved by the Invention

An object of the invention is to provide an organic electroluminescence device with improved performance. Another object of the invention is to provide an organic electroluminescence device with improved luminous efficiency. Still another object of the invention is to provide an organic electroluminescence device emitting light with a long lifetime. A further object of the invention is to provide an electronic device including the organic electroluminescence device.

### Means for Solving the Problem(s)

According to an aspect of the invention, there is provided an organic electroluminescence device including: an anode; a cathode; a first emitting layer provided between the anode and the cathode and containing a first compound; and a second emitting layer provided between the anode and the cathode and containing a second compound, in which at least one of the first emitting layer or the second emitting layer contains a compound having at least one deuterium atom, and at least one of the first emitting layer or the second emitting layer contains a compound having a fused ring that includes four or more rings.

According to another aspect of the invention, an electronic device including the organic electroluminescence device according to the above aspect of the invention is provided.

According to the above aspect of the invention, an organic electroluminescence device with enhanced performance can be provided. Further, according to the above aspect of the invention, an organic electroluminescence device with enhanced luminous efficiency can be provided. Furthermore, according to the above aspect of the invention, an organic electroluminescence device emitting light with a long lifetime can be provided. Moreover, according to the above aspect of the invention, an electronic device including the organic electroluminescence device can be provided.

### BRIEF EXPLANATION OF DRAWING(S)

FIG. 1 schematically shows an exemplary arrangement of an organic electroluminescence device according to an exemplary embodiment of the invention.

### DESCRIPTION OF EMBODIMENT(S)

### Definitions

Herein, a hydrogen atom includes isotope having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a deuterium.

Herein, the ring carbon atoms refer to the number of carbon atoms among atoms forming a ring of a compound (e.g., a monocyclic compound, fused-ring compound, cross-linking compound, carbon ring compound, and heterocyclic compound) in which the atoms are bonded to each other to form the ring. When the ring is substituted by a substituent(s), carbon atom(s) contained in the substituent(s) is not counted in the ring carbon atoms. Unless otherwise specified, the same applies to the “ring carbon atoms” described later. For instance, a benzene ring has 6 ring carbon atoms, a naphthalene ring has 10 ring carbon atoms, a pyridine ring has 5 ring carbon atoms, and a furan ring has 4 ring carbon atoms. Further, for instance, 9,9-diphenylfluorenyl group has 13 ring carbon atoms and 9,9′-spirobifluorenyl group has 25 ring carbon atoms.

When a benzene ring is substituted by a substituent in a form of, for instance, an alkyl group, the number of carbon atoms of the alkyl group is not counted in the number of the ring carbon atoms of the benzene ring. Accordingly, the benzene ring substituted by an alkyl group has 6 ring carbon atoms. When a naphthalene ring is substituted by a substituent in a form of, for instance, an alkyl group, the number of carbon atoms of the alkyl group is not counted in the number of the ring carbon atoms of the naphthalene ring. Accordingly, the naphthalene ring substituted by an alkyl group has 10 ring carbon atoms.

Herein, the ring atoms refer to the number of atoms forming a ring of a compound (e.g., a monocyclic compound, fused-ring compound, cross-linking compound, carbon ring compound, and heterocyclic compound) in which the atoms are bonded to each other to form the ring (e.g., monocyclic ring, fused ring, and ring assembly). Atom(s) not forming the ring (e.g., hydrogen atom(s) for saturating the valence of the atom which forms the ring) and atom(s) in a substituent by which the ring is substituted are not counted as the ring atoms. Unless otherwise specified, the same applies to the “ring atoms” described later. For instance, a pyridine ring has 6 ring atoms, a quinazoline ring has 10 ring atoms, and a furan ring has 5 ring atoms. For instance, the number of hydrogen atom(s) bonded to a pyridine ring or the number of atoms forming a substituent are not counted as the pyridine ring atoms. Accordingly, a pyridine ring bonded to a hydrogen atom(s) or a substituent(s) has 6 ring atoms. For instance, the hydrogen atom(s) bonded to carbon atom(s) of a quinazoline ring or the atoms forming a substituent are not counted as the quinazoline ring atoms. Accordingly, a quinazoline ring bonded to hydrogen atom(s) or a substituent(s) has 10 ring atoms.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group. Herein, “YY” is larger than “XX,” “XX” representing an integer of 1 or more and “YY” representing an integer of 2 or more.

Herein, “XX to YY atoms” in the description of “substituted or unsubstituted ZZ group having XX to YY atoms” represent atoms of an unsubstituted ZZ group and does not include atoms of a substituent(s) of the substituted ZZ group. Herein, “YY” is larger than “XX,” “XX” representing an integer of 1 or more and “YY” representing an integer of 2 or more.

Herein, an unsubstituted ZZ group refers to an “unsubstituted ZZ group” in a “substituted or unsubstituted ZZ group,” and a substituted ZZ group refers to a “substituted ZZ group” in a “substituted or unsubstituted ZZ group.”

Herein, the term “unsubstituted” used in a “substituted or unsubstituted ZZ group” means that a hydrogen atom(s) in the ZZ group is not substituted with a substituent(s). The hydrogen atom(s) in the “unsubstituted ZZ group” is protium, deuterium, or tritium.

Herein, the term “substituted” used in a “substituted or unsubstituted ZZ group” means that at least one hydrogen atom in the ZZ group is substituted with a substituent. Similarly, the term “substituted” used in a “BB group substituted by AA group” means that at least one hydrogen atom in the BB group is substituted with the AA group.

### Substituents Mentioned Herein

Substituents mentioned herein will be described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50, preferably 5 to 30, more preferably 5 to 18 ring atoms.

An “unsubstituted alkyl group” mentioned herein has, unless otherwise specified herein, 1 to 50, preferably 1 to 20, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50, preferably 2 to 20, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50, preferably 6 to 30, more preferably 6 to 18 ring carbon atoms.

An “unsubstituted divalent heterocyclic group” mentioned herein has, unless otherwise specified herein, 5 to 50, preferably 5 to 30, more preferably 5 to 18 ring atoms.

An “unsubstituted alkylene group” mentioned herein has, unless otherwise specified herein, 1 to 50, preferably 1 to 20, more preferably 1 to 6 carbon atoms.

### Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G11B). (Herein, an unsubstituted aryl group refers to an “unsubstituted aryl group” in a “substituted or unsubstituted aryl group”, and a substituted aryl group refers to a “substituted aryl group” in a “substituted or unsubstituted aryl group.”) A simply termed “aryl group” herein includes both of an “unsubstituted aryl group” and a “substituted aryl group.”

The “substituted aryl group” refers to a group derived by substituting at least one hydrogen atom in an “unsubstituted aryl group” with a substituent. Examples of the “substituted aryl group” include a group derived by substituting at least one hydrogen atom in the “unsubstituted aryl group” in the specific example group G1A below with a substituent, and examples of the substituted aryl group in the specific example group G1B below. It should be noted that the examples of the “unsubstituted aryl group” and the “substituted aryl group” mentioned herein are merely exemplary, and the “substituted aryl group” mentioned herein includes a group derived by further substituting a hydrogen atom bonded to a carbon atom of a skeleton of a “substituted aryl group” in the specific example group G1B below, and a group derived by further substituting a hydrogen atom of a substituent of the “substituted aryl group” in the specific example group G1B below.

### Unsubstituted Aryl Group (Specific Example Group G1A):

phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl group, benzofluoranthenyl group, perylenyl group, and a monovalent aryl group derived by removing one hydrogen atom from cyclic structures represented by formulae (TEMP-1) to (TEMP-15) below.

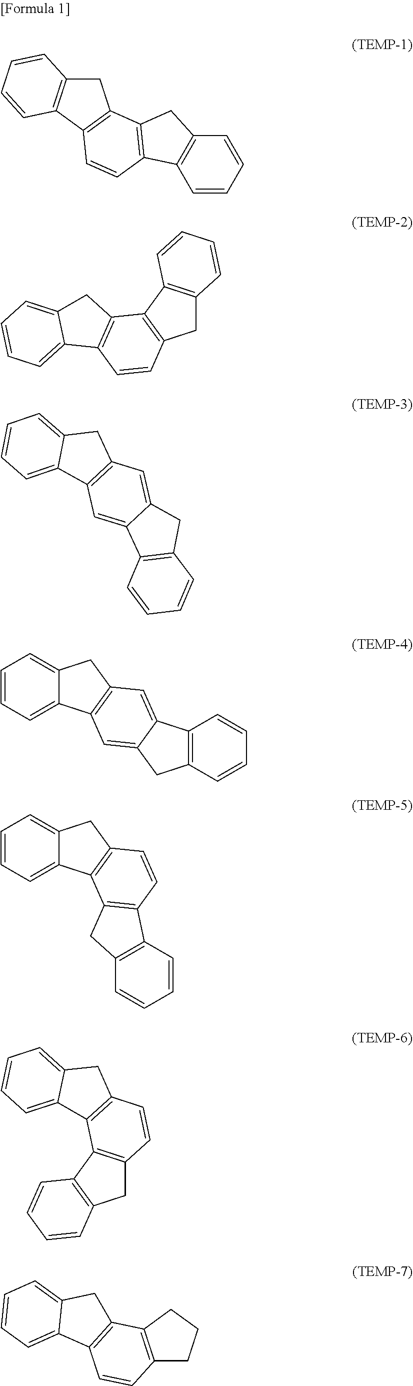

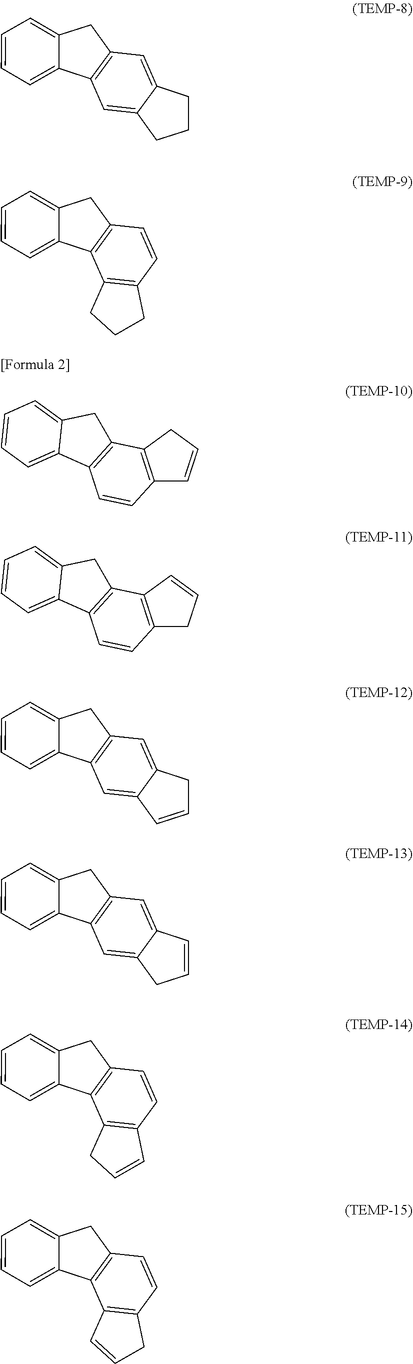

### Substituted Aryl Group (Specific Example Group G1B):

o-tolyl group, m-tolyl group, p-tolyl group, para-xylyl group, meta-xylyl group, ortho-xylyl group, para-isopropylphenyl group, meta-isopropylphenyl group, ortho-isopropylphenyl group, para-t-butylphenyl group, meta-t-butylphenyl group, ortho-t-butylphenyl group, 3,4,5-trimethylphenyl group, 9,9-dimethylfluorenyl group, 9,9-diphenylfluorenyl group, 9,9-bis(4-methylphenyl)fluorenyl group, 9,9-bis(4-isopropylphenyl)fluorenyl group, 9,9-bis(4-t-butylphenyl)fluorenyl group, cyanophenyl group, triphenylsilylphenyl group, trimethylsilylphenyl group, phenylnaphthyl group, naphthylphenyl group, and a group derived by substituting at least one hydrogen atom of a monovalent group derived from one of the cyclic structures represented by the formulae (TEMP-1) to (TEMP-15) with a substituent.

### Substituted or Unsubstituted Heterocyclic Group

The “heterocyclic group” mentioned herein refers to a cyclic group having at least one hetero atom in the ring atoms. Specific examples of the hetero atom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B). (Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.”) A simply termed “heterocyclic group” herein includes both of “unsubstituted heterocyclic group” and “substituted heterocyclic group.”

The “substituted heterocyclic group” refers to a group derived by substituting at least one hydrogen atom in an “unsubstituted heterocyclic group” with a substituent. Specific examples of the “substituted heterocyclic group” include a group derived by substituting at least one hydrogen atom in the “unsubstituted heterocyclic group” in the specific example group G2A below with a substituent, and examples of the substituted heterocyclic group in the specific example group G2B below. It should be noted that the examples of the “unsubstituted heterocyclic group” and the “substituted heterocyclic group” mentioned herein are merely exemplary, and the “substituted heterocyclic group” mentioned herein includes a group derived by further substituting a hydrogen atom bonded to a ring atom of a skeleton of a “substituted heterocyclic group” in the specific example group G2B below, and a group derived by further substituting a hydrogen atom of a substituent of the “substituted heterocyclic group” in the specific example group G2B below.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4) derived from the cyclic structures represented by formulae (TEMP-16) to (TEMP-33) below.

### Unsubstituted Heterocyclic Groups Including Nitrogen Atom (Specific Example Group G2A1):

pyrrolyl group, imidazolyl group, pyrazolyl group, triazolyl group, tetrazolyl group, oxazolyl group, isoxazolyl group, oxadiazolyl group, thiazolyl group, isothiazolyl group, thiadiazolyl group, pyridyl group, pyridazynyl group, pyrimidinyl group, pyrazinyl group, triazinyl group, indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, and diazacarbazolyl group.

### Unsubstituted Heterocyclic Groups Including Oxygen Atom (Specific Example Group G2A2):

furyl group, oxazolyl group, isoxazolyl group, oxadiazolyl group, xanthenyl group, benzofuranyl group, isobenzofuranyl group, dibenzofuranyl group, naphthobenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

### Unsubstituted Heterocyclic Groups Including Sulfur Atom (Specific Example Group G2A3):

thienyl group, thiazolyl group, isothiazolyl group, thiadiazolyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), and diazanaphthobenzothiophenyl group (diazanaphthobenzothienyl group).
   Monovalent Heterocyclic Groups Derived by Removing One Hydrogen Atom from Cyclic Structures Represented by Formulae (TEMP-16) to (TEMP-33) (Specific Example Group G2A4):

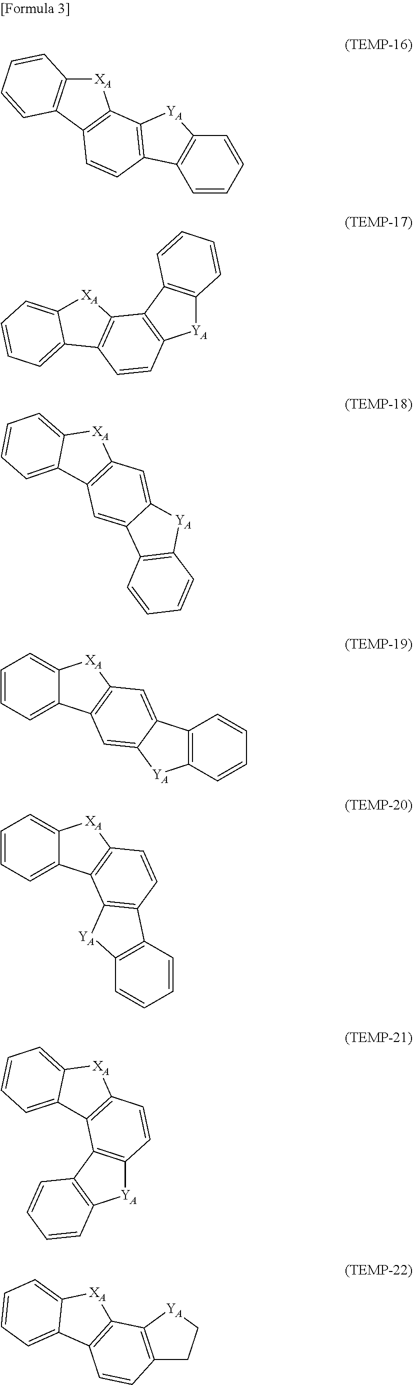

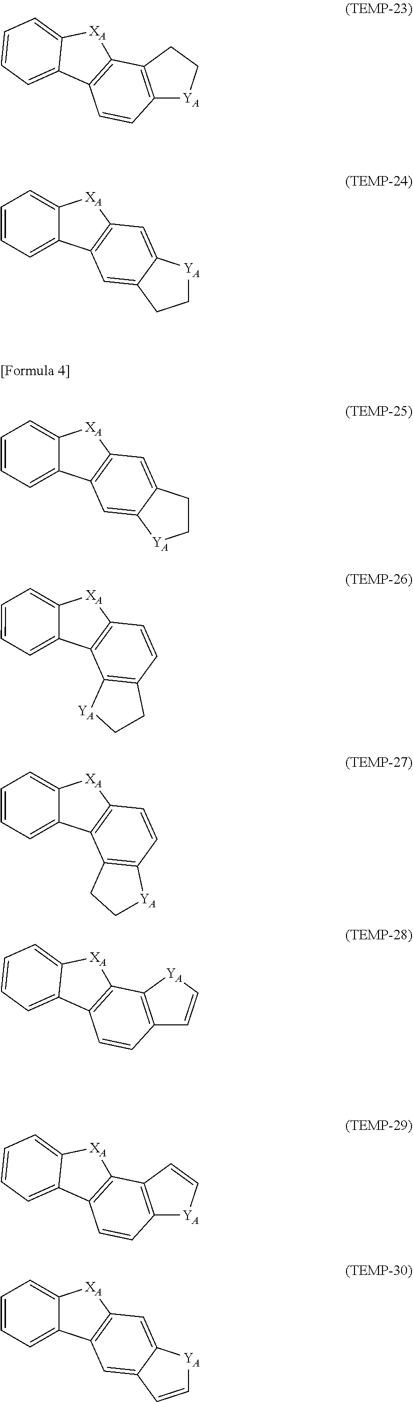

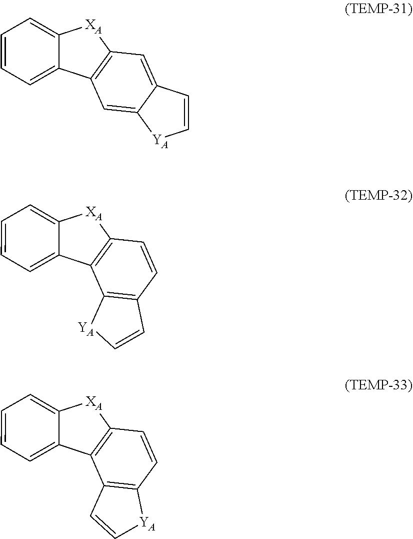

In the formulae (TEMP-16) to (TEMP-33), X_{A} and Y_{A} are each independently an oxygen atom, a sulfur atom, NH or CH₂, with a proviso that at least one of X_{A} or Y_{A} is an oxygen atom, a sulfur atom, or NH.

When at least one of X_{A} or Y_{A} in the formulae (TEMP-16) to (TEMP-33) is NH or CH₂, the monovalent heterocyclic groups derived from the cyclic structures represented by the formulae (TEMP-16) to (TEMP-33) include a monovalent group derived by removing one hydrogen atom from NH or CH₂.

### Substituted Heterocyclic Groups Including Nitrogen Atom (Specific Example Group G2B1):

(9-phenyl)carbazolyl group, (9-biphenylyl)carbazolyl group, (9-phenyl)phenylcarbazolyl group, (9-naphthyl)carbazolyl group, diphenylcarbazole-9-yl group, phenylcarbazole-9-yl group, methylbenzimidazolyl group, ethylbenzimidazolyl group, phenyltriazinyl group, biphenylyltriazinyl group, diphenyltriazinyl group, phenylquinazolinyl group, and biphenylquinazolinyl group.

### Substituted Heterocyclic Groups Including Oxygen Atom (Specific Example Group G2B2):

phenyldibenzofuranyl group, methyldibenzofuranyl group, t-butyldibenzofuranyl group, and monovalent residue of spiro[9H-xanthene-9,9′-[9H]fluorene].

### Substituted Heterocyclic Groups Including Sulfur Atom (Specific Example Group G2B3):

phenyldibenzothiophenyl group, methyldibenzothiophenyl group, t-butyldibenzothiophenyl group, and monovalent residue of spiro[9H-thioxanthene-9,9′-[9H]fluorene].
   Groups Obtained by Substituting at Least One Hydrogen Atom of Monovalent Heterocyclic Group Derived from Cyclic Structures Represented by Formulae (TEMP-16) to (TEMP-33) with Substituent (Specific Example Group G2B4):

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of X_{A} or Y_{A} in a form of NH, and a hydrogen atom of one of X_{A} and Y_{A} in a form of a methylene group (CH₂).

### Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B) below. (Herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.”) A simply termed “alkyl group” herein includes both of “unsubstituted alkyl group” and “substituted alkyl group.”

The “substituted alkyl group” refers to a group derived by substituting at least one hydrogen atom in an “unsubstituted alkyl group” with a substituent. Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below. Herein, the alkyl group for the “unsubstituted alkyl group” refers to a chain alkyl group. Accordingly, the “unsubstituted alkyl group” include linear “unsubstituted alkyl group” and branched “unsubstituted alkyl group.” It should be noted that the examples of the “unsubstituted alkyl group” and the “substituted alkyl group” mentioned herein are merely exemplary, and the “substituted alkyl group” mentioned herein includes a group derived by further substituting a hydrogen atom of a skeleton of the “substituted alkyl group” in the specific example group G3B, and a group derived by further substituting a hydrogen atom of a substituent of the “substituted alkyl group” in the specific example group G3B.

### Unsubstituted Alkyl Group (Specific Example Group G3A):

methyl group, ethyl group, n-propyl group, isopropyl group, n-butyl group, isobutyl group, s-butyl group, and t-butyl group.

### Substituted Alkyl Group (Specific Example Group G3B):

heptafluoropropyl group (including isomer thereof), pentafluoroethyl group, 2,2,2-trifluoroethyl group, and trifluoromethyl group.

### Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B). (Herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.”) A simply termed “alkenyl group” herein includes both of “unsubstituted alkenyl group” and “substituted alkenyl group.”

The “substituted alkenyl group” refers to a group derived by substituting at least one hydrogen atom in an “unsubstituted alkenyl group” with a substituent. Specific examples of the “substituted alkenyl group” include an “unsubstituted alkenyl group” (specific example group G4A) substituted by a substituent, and examples of the substituted alkenyl group (specific example group G4B) below. It should be noted that the examples of the “unsubstituted alkenyl group” and the “substituted alkenyl group” mentioned herein are merely exemplary, and the “substituted alkenyl group” mentioned herein includes a group derived by further substituting a hydrogen atom of a skeleton of the “substituted alkenyl group” in the specific example group G4B with a substituent, and a group derived by further substituting a hydrogen atom of a substituent of the “substituted alkenyl group” in the specific example group G4B with a substituent.

### Unsubstituted Alkenyl Group (Specific Example Group G4A):

vinyl group, allyl group, 1-butenyl group, 2-butenyl group, and 3-butenyl group.

### Substituted Alkenyl Group (Specific Example Group G4B):

1,3-butanedienyl group, 1-methylvinyl group, 1-methylallyl group, 1,1-dimethylallyl group, 2-methylallyl group, and 1,2-dimethylallyl group.

### Substituted or Unsubstituted Alkynyl Group

Specific examples (specific example group G5) of the “substituted or unsubstituted alkynyl group” mentioned herein include unsubstituted alkynyl groups (specific example group G5A) below. (Herein, an unsubstituted alkynyl group refers to an “unsubstituted alkynyl group” in a “substituted or unsubstituted alkynyl group.”) A simply termed “alkynyl group” herein includes both of “unsubstituted alkynyl group” and “substituted alkynyl group.”

The “substituted alkynyl group” refers to a group derived by substituting at least one hydrogen atom in an “unsubstituted alkynyl group” with a substituent. Specific examples of the “substituted alkynyl group” include a group derived by substituting at least one hydrogen atom of the “unsubstituted alkynyl group” (specific example group G5A) below with a substituent.

Unsubstituted Alkynyl Group (Specific Example Group G5A): ethynyl group.

### Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B). (Herein, an unsubstituted cycloalkyl group refers to an “unsubstituted cycloalkyl group” in a “substituted or unsubstituted cycloalkyl group,” and a substituted cycloalkyl group refers to a “substituted cycloalkyl group” in a “substituted or unsubstituted cycloalkyl group.”) A simply termed “cycloalkyl group” herein includes both of “unsubstituted cycloalkyl group” and “substituted cycloalkyl group.”

The “substituted cycloalkyl group” refers to a group derived by substituting at least one hydrogen atom of an “unsubstituted cycloalkyl group” with a substituent. Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below. It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by further substituting a hydrogen atom of a substituent of the “substituted cycloalkyl group” in the specific example group G6B with a substituent.

### Unsubstituted Cycloalkyl Group (Specific Example Group G6A):

cyclopropyl group, cyclobutyl group, cyclopentyl group, cyclohexyl group, 1-adamantyl group, 2-adamantyl group, 1-norbornyl group, and 2-norbornyl group.
   Substituted Cycloalkyl Group (Specific Example Group G6B): 4-methylcyclohexyl group.
   Group Represented by —Si(R₉₀₁)(R₉₀₂)(R₉₀₃)

Specific examples (specific example group G7) of the group represented herein by —Si(R₉₀₁)(R₉₀₂)(R₉₀₃) include: —Si(G1)(G1)(G1); —Si(G1)(G2)(G2); —Si(G1)(G1)(G2); —Si(G2)(G2)(G2); —Si(G3)(G3)(G3); and —Si(G6)(G6)(G6), where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6;
a plurality of G1 in —Si(G1)(G1)(G1) are mutually the same or different;
a plurality of G2 in —Si(G1)(G2)(G2) are mutually the same or different;
a plurality of G1 in —Si(G1)(G1)(G2) are mutually the same or different;
a plurality of G2 in —Si(G2)(G2)(G2) are mutually the same or different;
a plurality of G3 in —Si(G3)(G3)(G3) are mutually the same or different; and
a plurality of G6 in —Si(G6)(G6)(G6) are mutually the same or different.

### Group Represented by —O—(R₉₀₄)

Specific examples (specific example group G8) of a group represented by —O—(R₉₀₄) herein include: —O(G1); —O(G2); —O(G3); and —O(G6), where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.

### Group Represented by —S—(R₉₀₅)

Specific examples (specific example group G9) of a group represented herein by —S—(R₉₀₅) include: —S(G1); —S(G2); —S(G3); and —S(G6), where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.
   Group Represented by —N(R₉₀₆)(R₉₀₇)

Specific examples (specific example group G10) of a group represented herein by —N(R₉₀₆)(R₉₀₇) include: —N(G1)(G1); —N(G2)(G2); —N(G1)(G2); —N(G3)(G3); and —N(G6)(G6), where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6;
a plurality of G1 in —N(G1)(G1) are mutually the same or different;
a plurality of G2 in —N(G2)(G2) are mutually the same or different;
a plurality of G3 in —N(G3)(G3) are mutually the same or different; and
a plurality of G6 in —N(G6)(G6) are mutually the same or different.

### Halogen Atom

Specific examples (specific example group G11) of “halogen atom” mentioned herein include a fluorine atom, chlorine atom, bromine atom, and iodine atom.

### Substituted or Unsubstituted Fluoroalkyl Group

The “substituted or unsubstituted fluoroalkyl group” mentioned herein refers to a group derived by substituting at least one hydrogen atom bonded to at least one of carbon atoms forming an alkyl group in the “substituted or unsubstituted alkyl group” with a fluorine atom, and also includes a group (perfluoro group) derived by substituting all of hydrogen atoms bonded to carbon atoms forming the alkyl group in the “substituted or unsubstituted alkyl group” with fluorine atoms. An “unsubstituted fluoroalkyl group” has, unless otherwise specified herein, 1 to 50, preferably 1 to 30, more preferably 1 to 18 carbon atoms. The “substituted fluoroalkyl group” refers to a group derived by substituting at least one hydrogen atom in a “fluoroalkyl group” with a substituent. It should be noted that the examples of the “substituted fluoroalkyl group” mentioned herein include a group derived by further substituting at least one hydrogen atom bonded to a carbon atom of an alkyl chain of a “substituted fluoroalkyl group” with a substituent, and a group derived by further substituting at least one hydrogen atom of a substituent of the “substituted fluoroalkyl group” with a substituent. Specific examples of the “substituted fluoroalkyl group” include a group derived by substituting at least one hydrogen atom of the “alkyl group” (specific example group G3) with a fluorine atom.

### Substituted or Unsubstituted Haloalkyl Group

The “substituted or unsubstituted haloalkyl group” mentioned herein refers to a group derived by substituting at least one hydrogen atom bonded to carbon atoms forming the alkyl group in the “substituted or unsubstituted alkyl group” with a halogen atom, and also includes a group derived by substituting all hydrogen atoms bonded to carbon atoms forming the alkyl group in the “substituted or unsubstituted alkyl group” with halogen atoms. An “unsubstituted haloalkyl group” has, unless otherwise specified herein, 1 to 50, preferably 1 to 30, and more preferably 1 to 18 carbon atoms. The “substituted haloalkyl group” refers to a group derived by substituting at least one hydrogen atom in a “haloalkyl group” with a substituent. It should be noted that the examples of the “substituted haloalkyl group” mentioned herein include a group derived by further substituting at least one hydrogen atom bonded to a carbon atom of an alkyl chain of a “substituted haloalkyl group” with a substituent, and a group derived by further substituting at least one hydrogen atom of a substituent of the “substituted haloalkyl group” with a substituent. Specific examples of the “substituted haloalkyl group” include a group derived by substituting at least one hydrogen atom of the “alkyl group” (specific example group G3) with a halogen atom. The haloalkyl group is sometimes referred to as a halogenated alkyl group.

### Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3. An “unsubstituted alkoxy group” has, unless otherwise specified herein, 1 to 50, preferably 1 to 30, more preferably 1 to 18 carbon atoms.

### Substituted or Unsubstituted Alkylthio Group

Specific examples of a “substituted or unsubstituted alkylthio group” mentioned herein include a group represented by —S(G3), G3 being the “substituted or unsubstituted alkyl group” in the specific example group G3. An “unsubstituted alkylthio group” has, unless otherwise specified herein, 1 to 50, preferably 1 to 30, more preferably 1 to 18 carbon atoms.

### Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1. An “unsubstituted aryloxy group” has, unless otherwise specified herein, 6 to 50, preferably 6 to 30, more preferably 6 to 18 ring carbon atoms.

### Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1. An “unsubstituted arylthio group” has, unless otherwise specified herein, 6 to 50, preferably 6 to 30, more preferably 6 to 18 ring carbon atoms.

### Substituted or Unsubstituted Trialkylsilyl Group

Specific examples of a “trialkylsilyl group” mentioned herein include a group represented by —Si(G3)(G3)(G3), G3 being the “substituted or unsubstituted alkyl group” in the specific example group G3. The plurality of G3 in —Si(G3)(G3)(G3) are mutually the same or different. Each of the alkyl groups in the “trialkylsilyl group” has, unless otherwise specified herein, 1 to 50, preferably 1 to 20, more preferably 1 to 6 carbon atoms.

### Substituted or Unsubstituted Aralkyl Group

Specific examples of a “substituted or unsubstituted aralkyl group” mentioned herein include a group represented by (G3)-(G1), G3 being the “substituted or unsubstituted alkyl group” in the specific example group G3, G1 being the “substituted or unsubstituted aryl group” in the specific example group G1. Accordingly, the “aralkyl group” is a group derived by substituting a hydrogen atom of the “alkyl group” with a substituent in a form of the “aryl group,” which is an example of the “substituted alkyl group.” An “unsubstituted aralkyl group,” which is an “unsubstituted alkyl group” substituted by an “unsubstituted aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenylfluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl)carbazolyl group ((9-phenyl)carbazole-1-yl group, (9-phenyl)carbazole-2-yl group, (9-phenyl)carbazole-3-yl group, or (9-phenyl)carbazole-4-yl group), (9-biphenylyl)carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

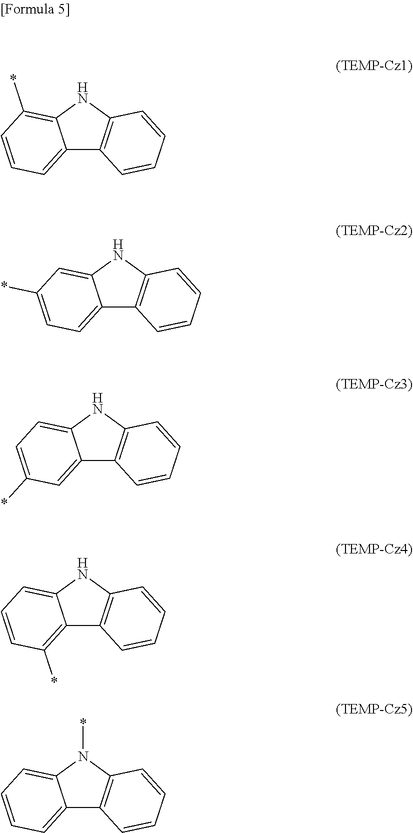 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

The (9-phenyl)carbazolyl group mentioned herein is, unless otherwise specified herein, specifically a group represented by one of formulae below.

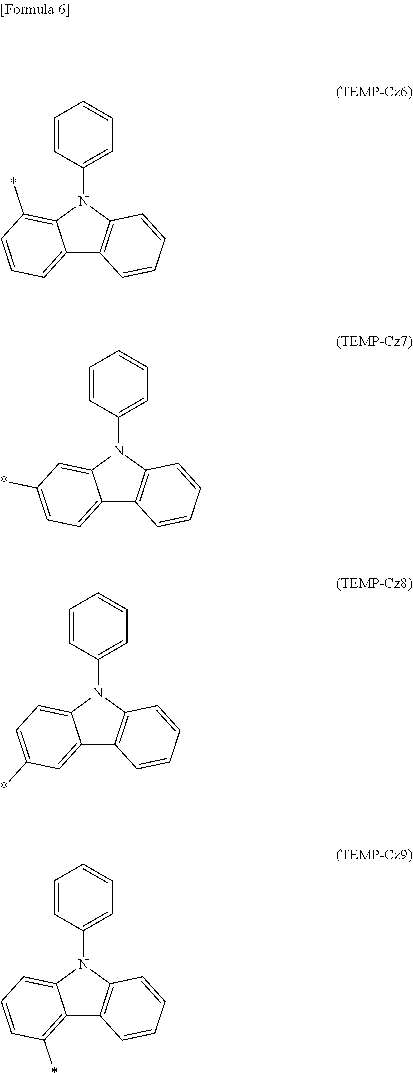 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the formulae (TEMP-Cz1) to (TEMP-Cz9), * represents a bonding position.

The dibenzofuranyl group and dibenzothiophenyl group mentioned herein are, unless otherwise specified herein, each specifically represented by one of formulae below.

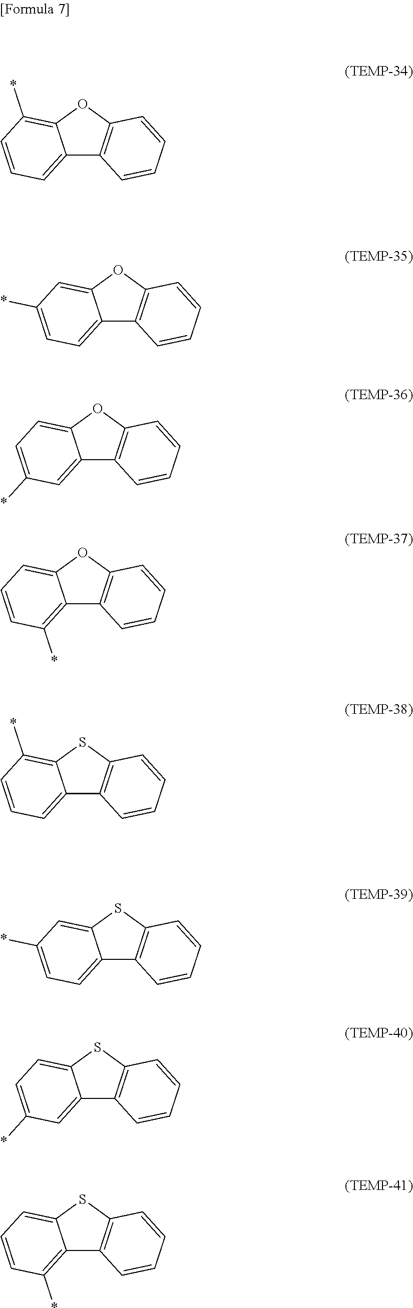

In the formulae (TEMP-34) to (TEMP-41), * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

### Substituted or Unsubstituted Arylene Group

The “substituted or unsubstituted arylene group” mentioned herein is, unless otherwise specified herein, a divalent group derived by removing one hydrogen atom on an aryl ring of the “substituted or unsubstituted aryl group.” Specific examples of the “substituted or unsubstituted arylene group” (specific example group G12) include a divalent group derived by removing one hydrogen atom on an aryl ring of the “substituted or unsubstituted aryl group” in the specific example group G1.

### Substituted or Unsubstituted Divalent Heterocyclic Group

The “substituted or unsubstituted divalent heterocyclic group” mentioned herein is, unless otherwise specified herein, a divalent group derived by removing one hydrogen atom on a heterocycle of the “substituted or unsubstituted heterocyclic group.” Specific examples of the “substituted or unsubstituted divalent heterocyclic group” (specific example group G13) include a divalent group derived by removing one hydrogen atom on a heterocyclic ring of the “substituted or unsubstituted heterocyclic group” in the specific example group G2.

### Substituted or Unsubstituted Alkylene Group

The “substituted or unsubstituted alkylene group” mentioned herein is, unless otherwise specified herein, a divalent group derived by removing one hydrogen atom on an alkyl chain of the “substituted or unsubstituted alkyl group.” Specific examples of the “substituted or unsubstituted alkylene group” (specific example group G14) include a divalent group derived by removing one hydrogen atom on an alkyl chain of the “substituted or unsubstituted alkyl group” in the specific example group G3.

The substituted or unsubstituted arylene group mentioned herein is, unless otherwise specified herein, preferably any one of groups represented by formulae (TEMP-42) to (TEMP-68) below.

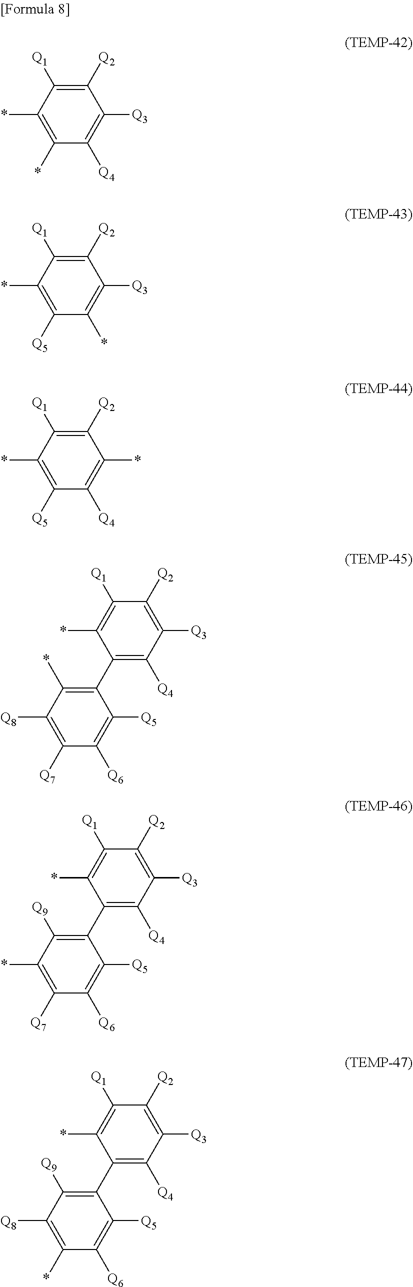

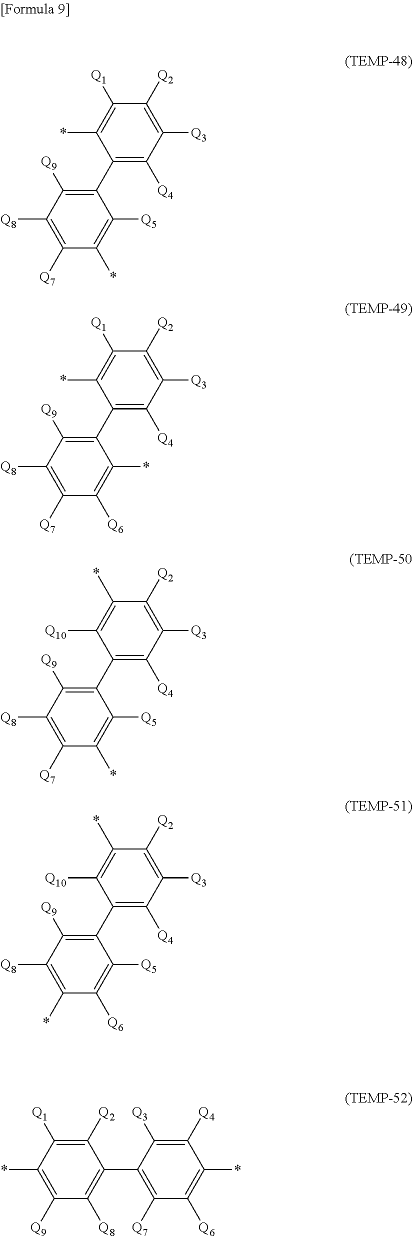

In the formulae (TEMP-42) to (TEMP-52), Q₁ to Q₁₀ each independently are a hydrogen atom or a substituent.

In the formulae (TEMP-42) to (TEMP-52), * represents a bonding position.

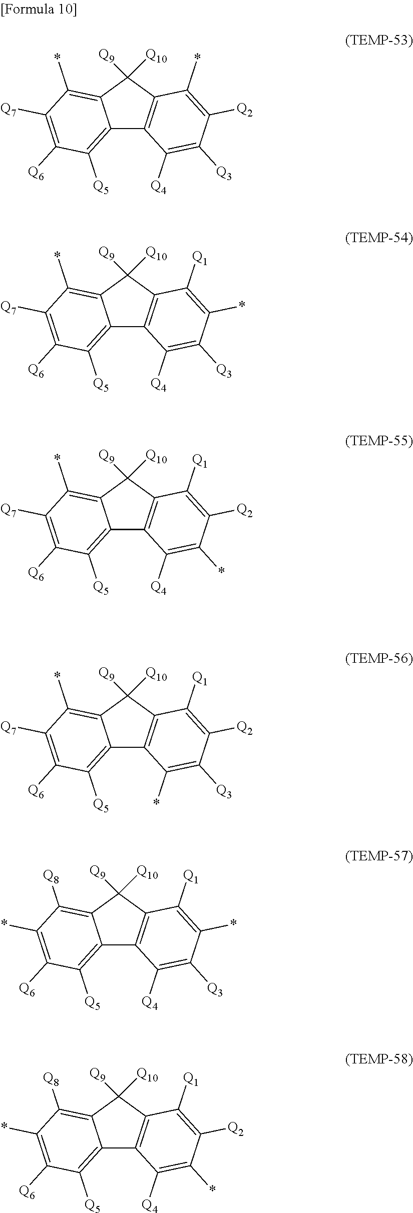

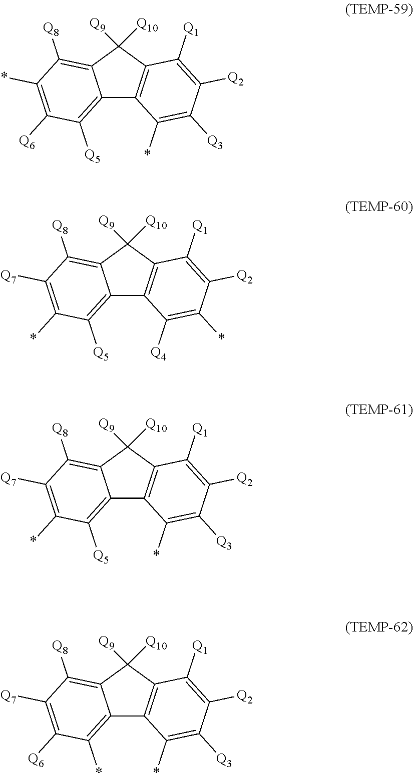

In the formulae (TEMP-53) to (TEMP-62), Q₁ to Q₁₀ each independently are a hydrogen atom or a substituent.

In the formulae, Q₉ and Q₁₀ may be mutually bonded through a single bond to form a ring.

In the formulae (TEMP-53) to (TEMP-62), * represents a bonding position.

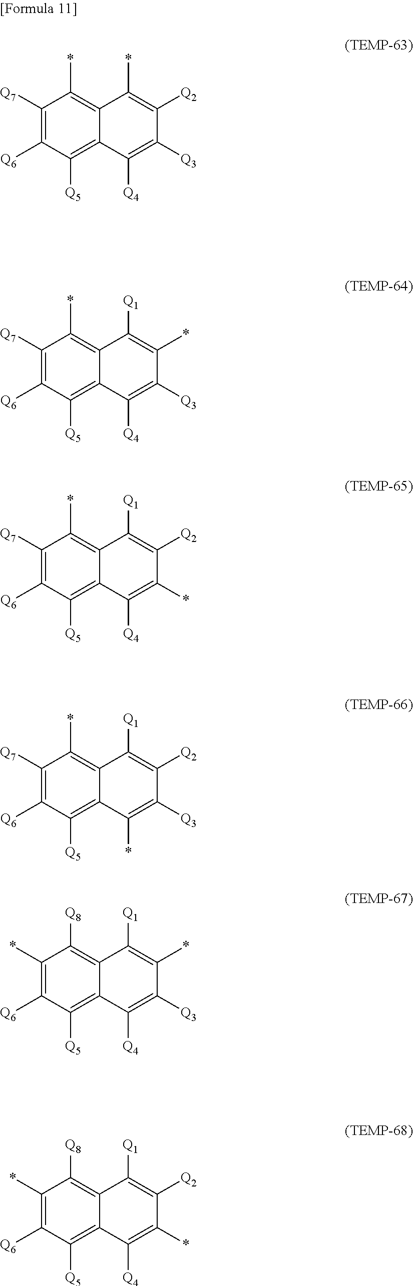

In the formulae (TEMP-63) to (TEMP-68), Q₁ to Q₈ each independently are a hydrogen atom or a substituent.

In the formulae (TEMP-63) to (TEMP-68), * represents a bonding position.

The substituted or unsubstituted divalent heterocyclic group mentioned herein is, unless otherwise specified herein, preferably a group represented by any one of formulae (TEMP-69) to (TEMP-102) below.

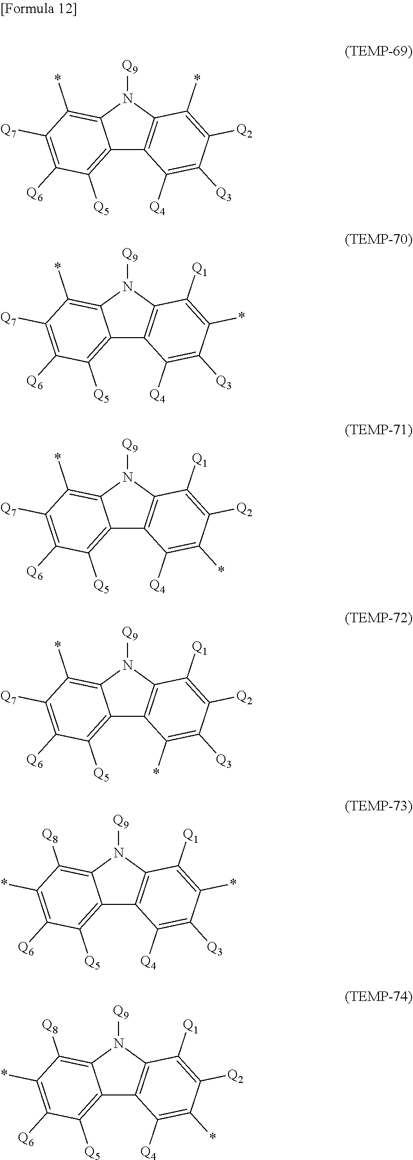

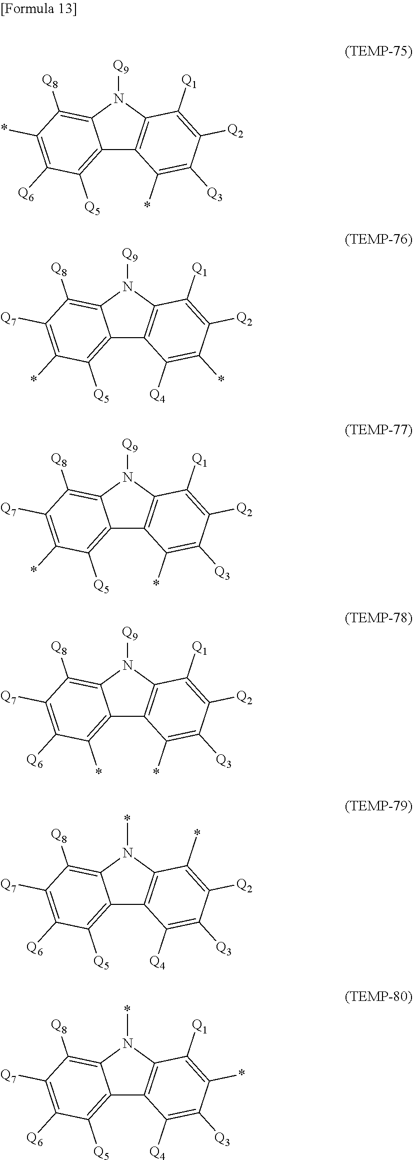

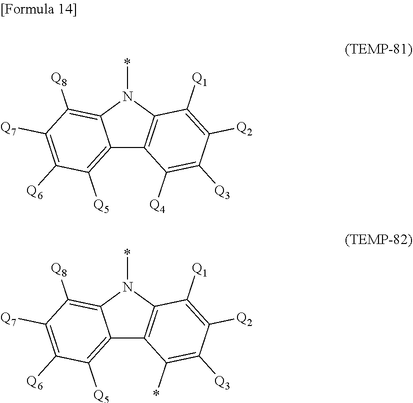

In the formulae (TEMP-69) to (TEMP-82), Q₁ to Q₉ each independently are a hydrogen atom or a substituent.

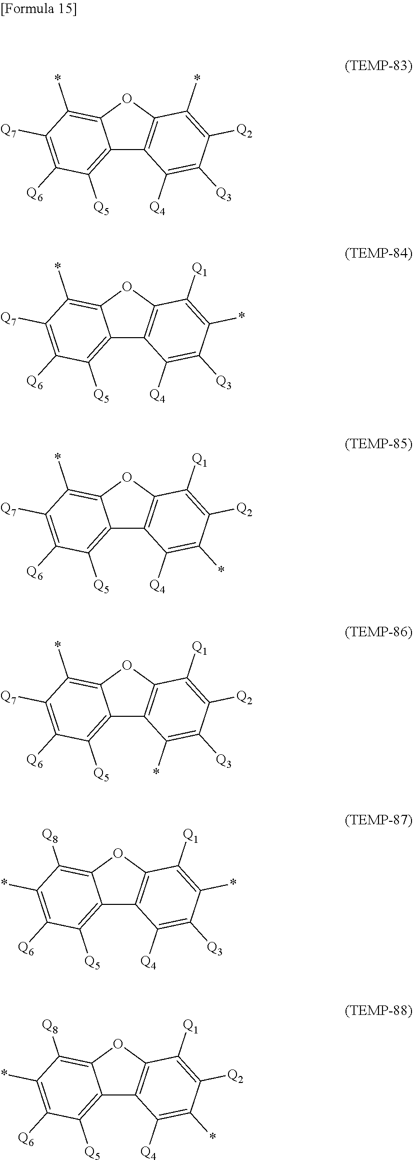

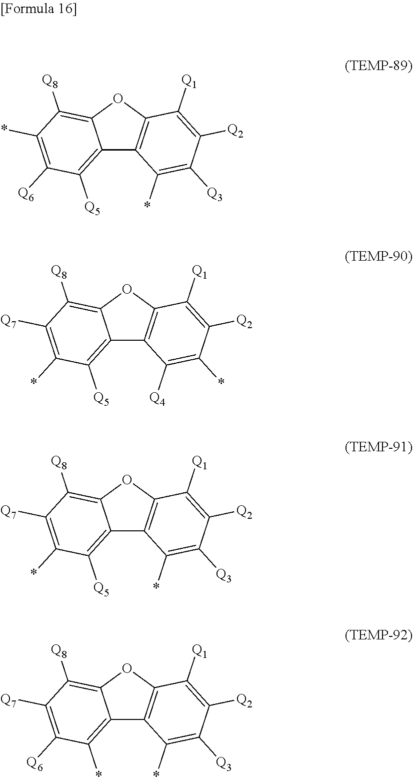

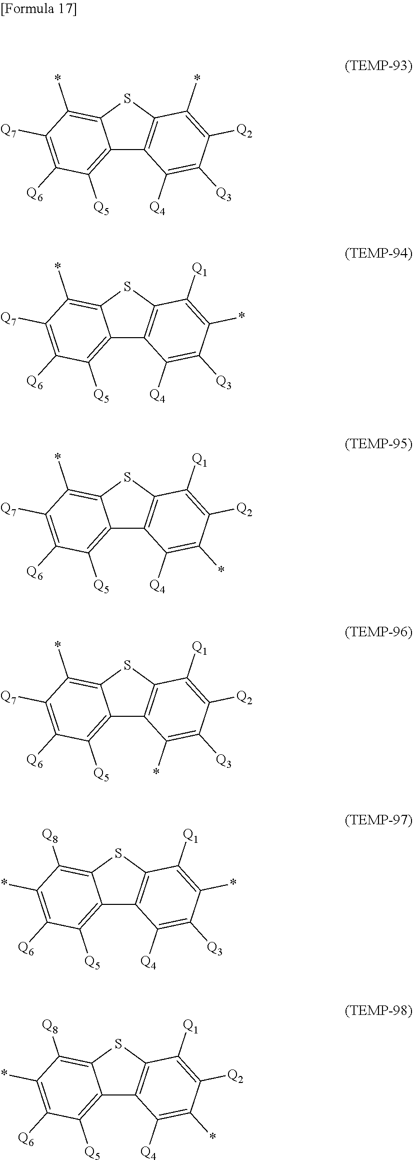

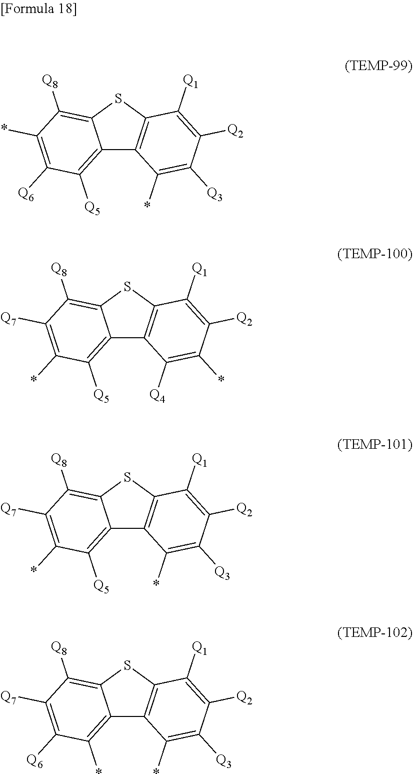

In the formulae (TEMP-83) to (TEMP-102), Q₁ to Qs each independently are a hydrogen atom or a substituent.

The substituent mentioned herein has been described above.

### Instance of “Bonded to Form Ring”

Instances where “at least one combination of adjacent two or more (of . . . ) are mutually bonded to form a substituted or unsubstituted monocyclic ring, mutually bonded to form a substituted or unsubstituted fused ring, or not mutually bonded” mentioned herein refer to instances where “at least one combination of adjacent two or more (of . . . ) are mutually bonded to form a substituted or unsubstituted monocyclic ring, “at least one combination of adjacent two or more (of . . . ) are mutually bonded to form a substituted or unsubstituted fused ring,” and “at least one combination of adjacent two or more (of . . . ) are not mutually bonded.”

Instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (these instances will be sometimes collectively referred to as an instance of “bonded to form a ring” hereinafter) will be described below. An anthracene compound having a basic skeleton in a form of an anthracene ring and represented by a formula (TEMP-103) below will be used as an example for the description.

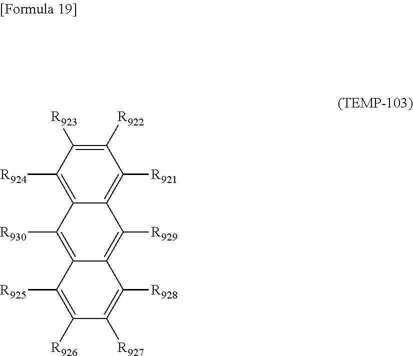

For instance, when “at least one combination of adjacent two or more of R₉₂₁ to R₉₃₀ are mutually bonded to form a ring,” the combination of adjacent ones of R₉₂₁ to R₉₃₀ (i.e. the combination at issue) is a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, or a combination of R₉₂₉ and R₉₂₁.

The term “at least one combination” means that two or more of the above combinations of adjacent two or more of R₉₂₁ to R₉₃₀ may simultaneously form rings. For instance, when R₉₂₁ and R₉₂₂ are mutually bonded to form a ring Q_{A} and R₉₂₅ and R₉₂₆ are simultaneously mutually bonded to form a ring Q_{B}, the anthracene compound represented by the formula (TEMP-103) is represented by a formula (TEMP-104) below.

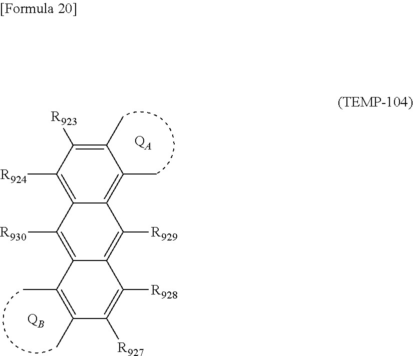

The instance where the “combination of adjacent two or more” form a ring means not only an instance where the “two” adjacent components are bonded but also an instance where adjacent “three or more” are bonded. For instance, R₉₂₁ and R₉₂₂ are mutually bonded to form a ring Q_{A} and R₉₂₂ and R₉₂₃ are mutually bonded to form a ring Qc, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below. In the formula (TEMP-105) below, the ring Q_{A} and the ring QC share R₉₂₂.

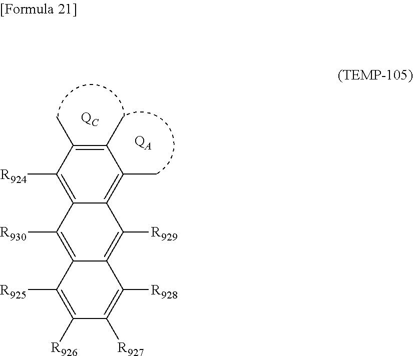

The formed “monocyclic ring” or “fused ring” may be, in terms of the formed ring in itself, a saturated ring or an unsaturated ring. When the “combination of adjacent two” form a “monocyclic ring” or a “fused ring,” the “monocyclic ring” or “fused ring” may be a saturated ring or an unsaturated ring. For instance, the ring Q_{A} and the ring Q_{B} formed in the formula (TEMP-104) are each independently a “monocyclic ring” or a “fused ring.” Further, the ring Q_{A} and the ring Qc formed in the formula (TEMP-105) are each a “fused ring.” The ring Q_{A} and the ring Qc in the formula (TEMP-105) are fused to form a fused ring. When the ring Q_{A} in the formula (TEMP-104) is a benzene ring, the ring Q_{A} is a monocyclic ring. When the ring Q_{A} in the formula (TEMP-104) is a naphthalene ring, the ring Q_{A} is a fused ring.

The “unsaturated ring” represents an aromatic hydrocarbon ring or an aromatic heterocycle. The “saturated ring” represents an aliphatic hydrocarbon ring or a non-aromatic heterocycle.

Specific examples of the aromatic hydrocarbon ring include a ring formed by terminating a bond of a group in the specific example of the specific example group G1 with a hydrogen atom.

Specific examples of the aromatic heterocycle include a ring formed by terminating a bond of an aromatic heterocyclic group in the specific example of the specific example group G2 with a hydrogen atom.

Specific examples of the aliphatic hydrocarbon ring include a ring formed by terminating a bond of a group in the specific example of the specific example group G6 with a hydrogen atom.

The phrase “to form a ring” herein means that a ring is formed only by a plurality of atoms of a basic skeleton, or by a combination of a plurality of atoms of the basic skeleton and one or more optional atoms. For instance, the ring Q_{A} formed by mutually bonding R₉₂₁ and R₉₂₂ shown in the formula (TEMP-104) is a ring formed by a carbon atom of the anthracene skeleton bonded to R₉₂₁, a carbon atom of the anthracene skeleton bonded to R₉₂₂, and one or more optional atoms. Specifically, when the ring Q_{A} is a monocyclic unsaturated ring formed by R₉₂₁ and R₉₂₂, the ring formed by a carbon atom of the anthracene skeleton bonded to R₉₂₁, a carbon atom of the anthracene skeleton bonded to R₉₂₂, and four carbon atoms is a benzene ring.

The “optional atom” is, unless otherwise specified herein, preferably at least one atom selected from the group consisting of a carbon atom, nitrogen atom, oxygen atom, and sulfur atom. A bond of the optional atom (e.g. a carbon atom and a nitrogen atom) not forming a ring may be terminated by a hydrogen atom or the like or may be substituted by an “optional substituent” described later. When the ring includes an optional element other than carbon atom, the resultant ring is a heterocycle.

The number of “one or more optional atoms” forming the monocyclic ring or fused ring is, unless otherwise specified herein, preferably in a range from 2 to 15, more preferably in a range from 3 to 12, further preferably in a range from 3 to 5.

Unless otherwise specified herein, the ring, which may be a “monocyclic ring” or “fused ring,” is preferably a “monocyclic ring.”

Unless otherwise specified herein, the ring, which may be a “saturated ring” or “unsaturated ring,” is preferably an “unsaturated ring.”

Unless otherwise specified herein, the “monocyclic ring” is preferably a benzene ring.

Unless otherwise specified herein, the “unsaturated ring” is preferably a benzene ring.

When “at least one combination of adjacent two or more” (of . . . ) are “mutually bonded to form a substituted or unsubstituted monocyclic ring” or “mutually bonded to form a substituted or unsubstituted fused ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “monocyclic ring” or the “fused ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

When the “saturated ring” or the “unsaturated ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

The above is the description for the instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (sometimes referred to as an instance of “bonded to form a ring”).

### Substituent for Substituted or Unsubstituted Group

In an exemplary embodiment herein, a substituent for the substituted or unsubstituted group (sometimes referred to as an “optional substituent” hereinafter) is, for instance, a group selected from the group consisting of an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, an unsubstituted aryl group having 6 to 50 ring carbon atoms, and an unsubstituted heterocyclic group having 5 to 50 ring atoms; R₉₀₁ to R₉₀₇ each independently are a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more R₉₀₃ are mutually the same or different;
when two or more R₉₀₄ are present, the two or more R₉₀₄ are mutually the same or different;
when two or more R₉₀₅ are present, the two or more R₉₀₅ are mutually the same or different;
when two or more R₉₀₆ are present, the two or more R₉₀₆ are mutually the same or different; and
when two or more R₉₀₇ are present, the two or more R₉₀₇ are mutually the same or different.

In an exemplary embodiment, a substituent for the substituted or unsubstituted group is selected from the group consisting of an alkyl group having 1 to 50 carbon atoms, an aryl group having 6 to 50 ring carbon atoms, and a heterocyclic group having 5 to 50 ring atoms.

In an exemplary embodiment, a substituent for the substituted or unsubstituted group is selected from the group consisting of an alkyl group having 1 to 18 carbon atoms, an aryl group having 6 to 18 ring carbon atoms, and a heterocyclic group having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted unsaturated five-membered ring, or a substituted or unsubstituted unsaturated six-membered ring, more preferably a benzene ring.

Unless otherwise specified herein, the optional substituent may further include a substituent. Examples of the substituent for the optional substituent are the same as the examples of the optional substituent.

Herein, numerical ranges represented by “AA to BB” represent a range whose lower limit is the value (AA) recited before “to” and whose upper limit is the value (BB) recited after “to.”

### First Exemplary Embodiment

### Organic Electroluminescence Device

An organic electroluminescence device according to a first exemplary embodiment includes: an anode; a cathode; a first emitting layer provided between the anode and the cathode and containing a first compound; and a second emitting layer provided between the anode and the cathode and containing a second compound, in which at least one of the first emitting layer or the second emitting layer contains a compound having at least one deuterium atom, and at least one of the first emitting layer or the second emitting layer contains a compound having a fused ring that includes four or more rings.

The fused ring that includes four or more rings means that the number of rings forming the fused ring is four or more. For example, phenyl-substituted anthracene has an anthracene ring having three rings and a benzene ring having one ring. The number of rings forming the fused ring, however, is three. Thus, phenyl-substituted anthracene does not have a fused ring that includes four or more rings.

When the organic EL device according to the exemplary embodiment contains a plurality of compounds each of which has a fused ring including four or more rings, the fused rings in the plurality of compounds are mutually the same or different.

The fused ring that includes four or more rings is preferably a substituted or unsubstituted aromatic hydrocarbon ring or a substituted or unsubstituted heterocycle, and more preferably a substituted or unsubstituted aromatic hydrocarbon ring. The four or more rings included in the fused ring are mutually the same or different. The four or more rings included in the fused ring are preferably each independently a five-membered ring or a six-membered ring. The fused ring that includes four or more rings may include both a five-membered ring and a six-membered ring. The fused ring that includes four or more rings includes, for instance, fluoranthene.

In the organic electroluminescence device according to the exemplary embodiment, at least one of the first emitting layer or the second emitting layer preferably contains a compound having a fused ring that includes 4 or more and 14 or less rings, and more preferably contains a compound having a fused ring that includes 4 or more and 10 or less rings.

In the organic electroluminescence device according to the exemplary embodiment, a structure of the first compound is the same as or different from a structure of the second compound, preferably the first compound and the second compound are different in structure.

In the organic electroluminescence device according to the exemplary embodiment, at least one of the first emitting layer or the second emitting layer preferably contains a compound having at least one deuterium atom and having a fused ring that includes four or more rings.

In the organic electroluminescence device according to the exemplary embodiment, at least the first emitting layer preferably contains a compound having at least one deuterium atom.

In the organic electroluminescence device according to the exemplary embodiment, the first compound also preferably has at least one deuterium atom.

In the organic electroluminescence device according to the exemplary embodiment, the first compound also preferably has a fused ring that includes four or more rings.

In the organic electroluminescence device according to the exemplary embodiment, the first compound also preferably has at least one deuterium atom and a fused ring that includes four or more rings.

In the organic electroluminescence device according to the exemplary embodiment, the second compound also preferably has at least one deuterium atom.

In the organic electroluminescence device according to the exemplary embodiment, the second compound also preferably has a fused ring that includes four or more rings.

In the organic electroluminescence device according to the exemplary embodiment, the second compound also preferably has at least one deuterium atom and a fused ring that includes four or more rings.

In the organic electroluminescence device according to the exemplary embodiment, the first compound in the first emitting layer and the second compound in the second emitting layer preferably satisfy any of conditions for Device A1 to Device A9 shown in Table 1 below.

TABLE 1

| | First emitting layer | | Second emitting layer | |
|---|---|---|---|---|
| | First compound | | Second compound | |
| | Having fused ring | | Having fused ring | |
| Organic EL | that includes four | Having at least one | that includes four | Having at least one |
| device | or more rings | deuterium atom | or more rings | deuterium atom |
| Device A1 | Y | Y | Y | Y |
| Device A2 | Y | Y | Y | N |
| Device A3 | Y | Y | N | Y |
| Device A4 | Y | Y | N | N |
| Device A5 | Y | N | Y | Y |
| Device A6 | Y | N | N | Y |
| Device A7 | N | Y | Y | Y |
| Device A8 | N | Y | Y | N |
| Device A9 | N | N | Y | Y |

In columns of “Having fused ring that includes four or more rings” of Table 1, “Y” means that a compound has a fused ring that includes four or more rings, and “N” means that a compound does not have a fused ring that includes four or more rings.

In columns of “Having at least one deuterium atom” of Table 1, “Y” means that a compound has at least one deuterium atom, and “N” means that a compound has no deuterium atom.

For instance, Device A1 of Table 1 means that the first emitting layer contains the first compound having at least one deuterium atom and a fused ring that includes four or more rings, and the second emitting layer contains the second compound having at least one deuterium atom and a fused ring that includes four or more rings.

For instance, Device A3 of Table 1 means that the first emitting layer contains the first compound having at least one deuterium atom and a fused ring that includes four or more rings, and the second emitting layer contains the second compound not having a fused ring that includes four or more rings and having at least one deuterium atom.

In the organic electroluminescence device according to the exemplary embodiment, a compound not having a fused ring that includes four or more rings is at least one of a compound having a fused ring that includes two or three rings or a compound having a monocyclic ring formed by one ring.

The fused ring that includes two or three rings and the monocyclic ring are preferably each independently a substituted or unsubstituted aromatic hydrocarbon ring or a substituted or unsubstituted heterocycle, and more preferably each independently a substituted or unsubstituted aromatic hydrocarbon ring. The two or three rings included in the fused ring are mutually the same or different. The two or three rings included in the fused ring and the monocyclic ring are preferably each independently a five-membered ring or a six-membered ring.

In the organic electroluminescence device according to the exemplary embodiment, a compound not having a fused ring that includes four or more rings is preferably a compound having a fused ring that includes two or three rings, and also preferably a compound having a monocyclic ring and a fused ring that includes two or three rings.

In the organic electroluminescence device according to the exemplary embodiment, it is also preferable that one of the first compound and the second compound substantially has no deuterium atom.

In an exemplary embodiment, only one of the first emitting layer and the second emitting layer contains a compound having at least one deuterium atom, and the other of the first emitting layer and the second emitting layer does not substantially contain a compound having a deuterium atom.

Here, “emitting layer does not substantially contain a compound having a deuterium atom” means that the emitting layer contains no deuterium atom or that the emitting layer is allowed to contain a deuterium atom approximately at the natural abundance ratio. The natural abundance ratio of the deuterium atom (mole fraction or atomic fraction) is, for instance, 0.015% or less.

That is, “emitting layer contains a compound having at least one deuterium atom” means that the emitting layer contains a compound having a deuterium atom at a content exceeding the natural abundance ratio.

Whether the compound has a deuterium atom is verified by mass spectrometry or ¹H-NMR spectrometry. A bonding position of a deuterium atom in the compound is specified by the ¹H-NMR spectrometry. Details are described below.

Mass spectrometry is performed on a target compound. When a molecular weight of the target compound is increased by, for example, one as compared with a related compound in which all the hydrogen atoms in the target compound are replaced by protium atoms, it can be determined that the target compound has a deuterium atom. Further, since a signal of a deuterium atom does not appear in ¹H-NMR spectrometry, the number of deuterium atoms in a molecule can be determined by an integral value obtained by performing ¹H-NMR spectrometry on the target compound. Furthermore, a bonding position of a deuterium atom can be determined by conducting ¹H-NMR spectrometry on the target compound to perform signal assignment.

In the organic electroluminescence device according to the exemplary embodiment, the first compound preferably has at least one skeleton selected from the group consisting of a pyrene skeleton, benzanthracene skeleton, xanthene skeleton, chrysene skeleton, fluoranthene skeleton, benzofluoranthene skeleton, triphenylene skeleton, benzoxanthene skeleton, benzophenanthrene skeleton, and benzochrysene skeleton. Each of the above skeletons may have a substituent.

In the organic electroluminescence device according to the exemplary embodiment, the second compound preferably has at least one skeleton selected from the group consisting of a pyrene skeleton, benzanthracene skeleton, xanthene skeleton, chrysene skeleton, fluoranthene skeleton, benzofluoranthene skeleton, triphenylene skeleton, benzoxanthene skeleton, benzophenanthrene skeleton, and benzochrysene skeleton. Each of the above skeletons may have a substituent.

In an exemplary embodiment, the first compound and the second compound have or do not have the same skeleton.

In the organic electroluminescence device according to the exemplary embodiment, the compound having the fused ring that includes four or more rings preferably has no anthracene skeleton.

### Compound Represented by Formula (1)

In an exemplary embodiment, the compound having the fused ring that includes four or more rings is a compound represented by a formula (1) below and having at least one group represented by a formula (11) below.

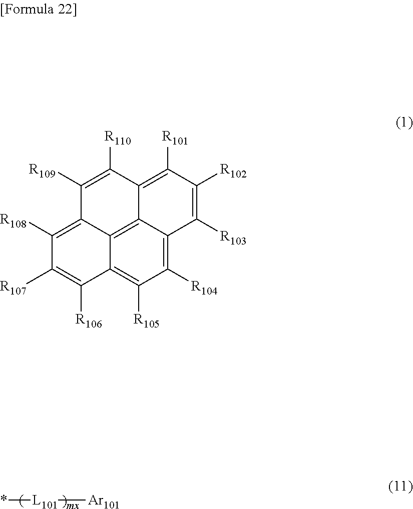

In the formula (1)
R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);
at least one of R₁₀₁ to R₁₁₀ is a group represented by the formula (11);
when a plurality of groups represented by the formula (11) are present, the plurality of groups represented by the formula (11) are mutually the same or different;
L₁₀₁ is a single bond, a substituted or unsubstituted arylene group having 6 to 50 ring carbon atoms, or a substituted or unsubstituted divalent heterocyclic group having 5 to 50 ring atoms,
Ar₁₀₁ is a substituted or unsubstituted aryl group having 6 to 50 ring carbon atoms, or a substituted or unsubstituted heterocyclic group having 5 to 50 ring atoms;
mx is 0, 1, 2, 3, 4, or 5;
when two or more L₁₀₁ are present, the two or more L₁₀₁ are mutually the same or different;
when two or more Ar₁₀₁ are present, the two or more Ar₁₀₁ are mutually the same or different; and
* in the formula (11) represents a bonding position to a pyrene ring in the formula (1).

In the compound represented by the formula (1), R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In the organic electroluminescence device according to the exemplary embodiment, at least one of R₁₀₁ to R₁₁₀ not being the group represented by the formula (11) is also preferably a deuterium atom.

In the organic electroluminescence device according to the exemplary embodiment, L₁₀₁ also preferably has at least one deuterium atom.

In the organic electroluminescence device according to the exemplary embodiment, Ar₁₀₁ also preferably has at least one deuterium atom.

In the organic EL device according to the exemplary embodiment, the group represented by the formula (11) is preferably a group represented by a formula (111) below.

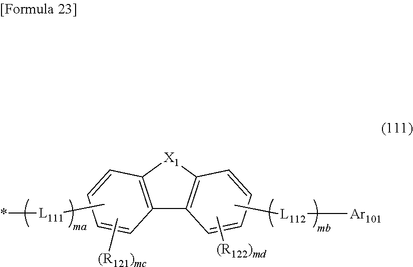

In the formula (111):
X₁ is CR₁₂₃R₁₂₄, an oxygen atom, a sulfur atom, or NR₁₂₅;
L₁₁₁ and L₁₁₂ are each independently a single bond, a substituted or unsubstituted arylene group having 6 to 50 ring carbon atoms, or a substituted or unsubstituted divalent heterocyclic group having 5 to 50 ring atoms;
ma is 0, 1, 2, 3, or 4;
mb is 0, 1, 2, 3, or 4;
ma+mb is 0, 1, 2, 3, or 4;
Ar₁₀₁ represents the same as Ar₁₀₁ in the formula (11);
R₁₂₁, R₁₂₂, R₁₂₃, R₁₂₄ and R₁₂₅ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
mc is 3;
three R₁₂₁ are mutually the same or different;
md is 3; and
three R₁₂₂ are mutually the same or different.

Among positions *1 to *8 of carbon atoms in a cyclic structure represented by a formula (111a) below in a group represented by the formula (111), L₁₁₁ is bonded to one of the positions *1 to *4, R₁₂₁ is bonded to each of three positions of the rest of *1 to *4, L₁₁₂ is bonded to one of the positions *5 to *8, and R₁₂₂ is bonded to each of three positions of the rest of *5 to *8.

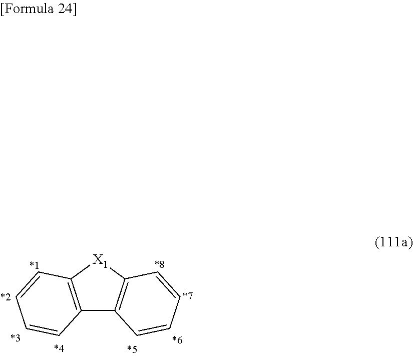

For instance, in the group represented by the formula (111), when L₁₁₁ is bonded to a carbon atom at a position *2 in the cyclic structure represented by the formula (111a) and L₁₁₂ is bonded to a carbon atom at a position *7 in the cyclic structure represented by the formula (111a), the group represented by the formula (111) is represented by a formula (111b) below.

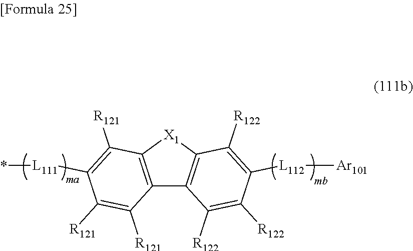

In the formula (111b):
X₁, L₁₁₁, L₁₁₂, ma, mb, Ar₁₀₁, R₁₂₁, R₁₂₂, R₁₂₃, R₁₂₄, and R₁₂₅ each independently represent the same as X₁, L₁₁₁, L₁₁₂, ma, mb, Ar₁₀₁, R₁₂₁, R₁₂₂, R₁₂₃, R₁₂₄, and R₁₂₅ in the formula (111);
a plurality of R₁₂₁ are mutually the same or different; and
a plurality of R₁₂₂ are mutually the same or different.

In the organic EL device according to the exemplary embodiment, the group represented by the formula (111) is preferably a group represented by the formula (111b).

In the organic EL device according to the exemplary embodiment, it is preferable that ma is 0, 1, or 2 and mb is 0, 1, or 2.

In the organic EL device according to the exemplary embodiment, it is preferable that ma is 0 or 1 and mb is 0 or 1.

In the organic EL device according to the exemplary embodiment, Ar₁₀₁ is preferably a substituted or unsubstituted aryl group having 6 to 50 ring carbon atoms.

In the organic EL device according to the exemplary embodiment, Ar₁₀₁ is preferably a substituted or unsubstituted phenyl group, a substituted or unsubstituted naphthyl group, a substituted or unsubstituted biphenyl group, a substituted or unsubstituted terphenyl group, a substituted or unsubstituted pyrenyl group, a substituted or unsubstituted phenanthryl group, or a substituted or unsubstituted fluorenyl group.

In the organic EL device according to the exemplary embodiment, Ar₁₀₁ is also preferably a group represented by a formula (12), a formula (13), or a formula (14) below.

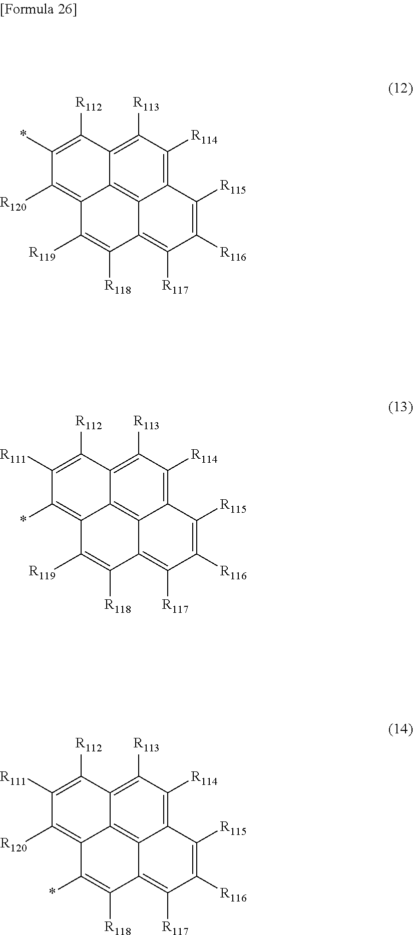

In the formulae (12), (13), and (14):
R₁₁₁ to R₁₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
* in the formulae (12), (13) and (14) represents a bonding position to L₁₀₁ in the formula (11), or a bonding position to L₁₁₂ in the formula (111) or (111 b).

In the organic EL device according to the exemplary embodiment, the compound represented by the formula (1) is preferably represented by a formula (101) below.

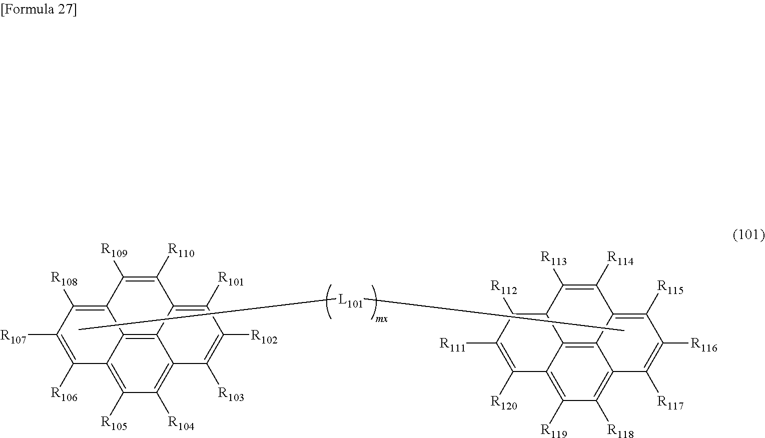

In the formula (101):
R₁₀₁ to R₁₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
one of R₁₀₁ to R₁₁₀ represents a bonding position to L₁₀₁, and one of R₁₁₁ to R₁₂₀ represents a bonding position to L₁₀₁;
L₁₀₁ is a single bond, a substituted or unsubstituted arylene group having 6 to 50 ring carbon atoms, or a substituted or unsubstituted divalent heterocyclic group having 5 to 50 ring atoms;
mx is 0, 1, 2, 3, 4, or 5; and
when two or more L₁₀₁ are present, the two or more L₁₀₁ are mutually the same or different.

In a compound represented by the formula (101), it is preferable that:
R₁₀₁ to R₁₁₀ and R₁₁₁ to R₁₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
one of R₁₀₁ to R₁₁₀ represents a bonding position to L₁₀₁, and one of R₁₁₁ to R₁₂₀ represents a bonding position to L₁₀₁;
L₁₀₁ is a single bond, a substituted or unsubstituted arylene group having 6 to 24 ring carbon atoms, or a substituted or unsubstituted divalent heterocyclic group having 5 to 24 ring atoms;
mx is 1, 2, 3, 4, or 5; and
when two or more L₁₀₁ are present, the two or more L₁₀₁ are mutually the same or different.

In the organic EL device according to the exemplary embodiment, the compound represented by the formula (1) is preferably represented by a formula (1010), a formula (1011), a formula (1012), a formula (1013), a formula (1014), or a formula (1015) below.

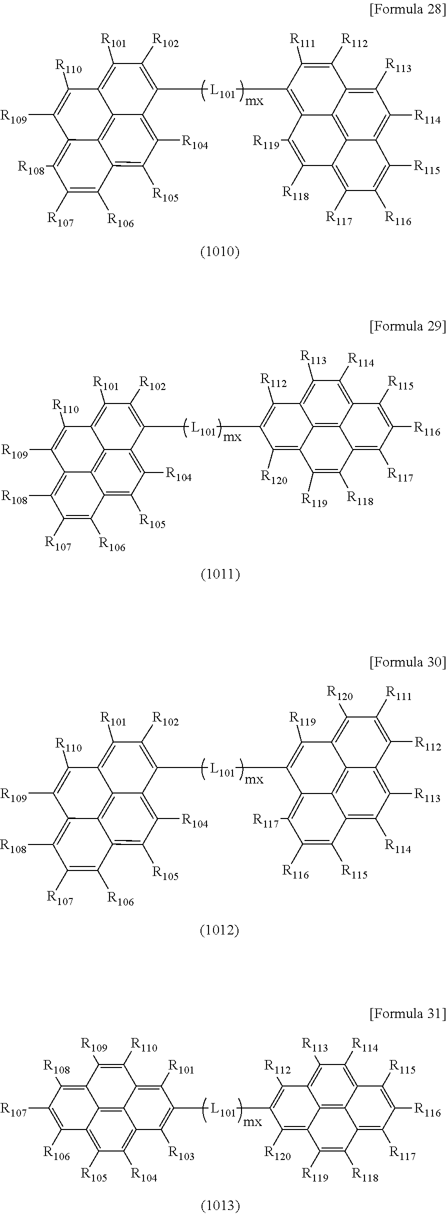

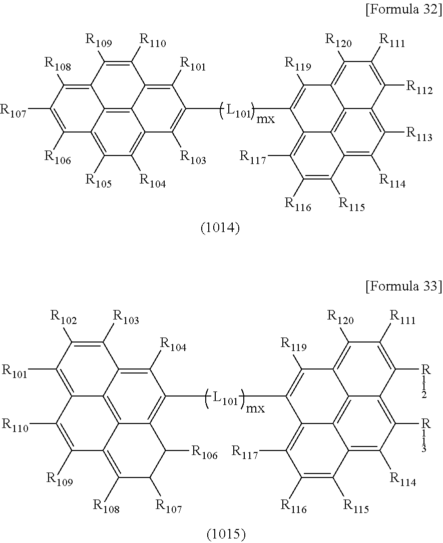

In the formulae (1010) to (1015):
R₁₀₁ to R₁₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms,
L₁₀₁ is a single bond, a substituted or unsubstituted arylene group having 6 to 50 ring carbon atoms, or a substituted or unsubstituted divalent heterocyclic group having 5 to 50 ring atoms;
mx is 0, 1, 2, 3, 4, or 5; and
when two or more L₁₀₁ are present, the two or more L₁₀₁ are mutually the same or different.

The compound represented by the formula (1010) corresponds to a compound, in which R₁₀₃ represents a bonding position to L₁₀₁ and R₁₂₀ represents a bonding position to L₁₀₁.

The compound represented by the formula (1011) corresponds to a compound, in which R₁₀₃ represents a bonding position to L₁₀₁ and R₁₁₁ represents a bonding position to L₁₀₁.

The compound represented by the formula (1012) corresponds to a compound, in which R₁₀₃ represents a bonding position to L₁₀₁ and R₁₁₈ represents a bonding position to L₁₀₁.

The compound represented by the formula (1013) corresponds to a compound, in which R₁₀₂ represents a bonding position to L₁₀₁ and R₁₁₁ represents a bonding position to L₁₀₁.

The compound represented by the formula (1014) corresponds to a compound, in which R₁₀₂ represents a bonding position to L₁₀₁ and R₁₁₈ represents a bonding position to L₁₀₁.

The compound represented by the formula (1015) corresponds to a compound, in which R₁₀₅ represents a bonding position to L₁₀₁ and R₁₁₈ represents a bonding position to L₁₀₁.

In the organic EL device according to the exemplary embodiment, the compound represented by the formula (1) is preferably represented by the formula (1010).

In the organic EL device according to the exemplary embodiment, R₁₀₁ to R₁₁₀ not being the bonding position to L₁₀₁ are preferably each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the organic EL device according to the exemplary embodiment, R₁₀₁ to R₁₁₀ not being the bonding position to L₁₀₁ are preferably each independently a hydrogen atom, a substituted or unsubstituted alkyl group having 1 to 50 carbon atoms, or a substituted or unsubstituted cycloalkyl group having 3 to 50 ring carbon atoms.

In the organic EL device according to the exemplary embodiment, R₁₀₁ to R₁₁₀ not being the bonding position to L₁₀₁ are each preferably a hydrogen atom.

In the organic EL device according to the exemplary embodiment, R₁₁₁ to R₁₂₀ not being the bonding position to L₁₀₁ are preferably each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the organic EL device according to the exemplary embodiment, R₁₁₁ to R₁₂₀ not being the bonding position to L₁₀₁ are preferably each independently a hydrogen atom, a substituted or unsubstituted alkyl group having 1 to 50 carbon atoms, or a substituted or unsubstituted cycloalkyl group having 3 to 50 ring carbon atoms.

In the organic EL device according to the exemplary embodiment, R₁₁₁ to R₁₂₀ not being the bonding position to L₁₀₁ are each preferably a hydrogen atom.

In the organic EL device according to the exemplary embodiment, L₁₀₁ is preferably a single bond, or a substituted or unsubstituted arylene group having 6 to 50 ring carbon atoms.

In the organic EL device according to the exemplary embodiment, L₁₀₁ is also preferably a single bond, a substituted or unsubstituted arylene group having 6 to 18 ring carbon atoms, or a substituted or unsubstituted divalent heterocyclic group having 5 to 18 ring atoms.

In the organic EL device according to the exemplary embodiment, L₁₀₁ is also preferably a single bond, or a substituted or unsubstituted arylene group having 6 to 18 ring carbon atoms.

In the organic EL device according to the exemplary embodiment, L₁₀₁ is also preferably a substituted or unsubstituted arylene group having 6 to 18 ring carbon atoms.

In the organic EL device according to the exemplary embodiment, L₁₀₁ is also preferably a single bond, a substituted or unsubstituted arylene group having 6 to 13 ring carbon atoms, or a substituted or unsubstituted divalent heterocyclic group having 5 to 13 ring atoms.

In the organic EL device according to the exemplary embodiment, L₁₀₁ is also preferably a single bond, or a substituted or unsubstituted arylene group having 6 to 13 ring carbon atoms.

In the organic EL device according to the exemplary embodiment, L₁₀₁ is also preferably a substituted or unsubstituted arylene group having 6 to 13 ring carbon atoms.

In the organic EL device according to the exemplary embodiment, mx is also preferably 1, 2, or 3.

In the organic EL device according to the exemplary embodiment, mx is also preferably 1 or 2.

In the organic EL device according to the exemplary embodiment, it is also preferable that mx is 1, 2, or 3; and L₁₀₁ is a substituted or unsubstituted arylene group having 6 to 18 ring carbon atoms, or a substituted or unsubstituted divalent heterocyclic group having 5 to 18 ring atoms.

In the organic EL device according to the exemplary embodiment, it is also preferable that mx is 1 or 2; and L₁₀₁ is a substituted or unsubstituted arylene group having 6 to 18 ring carbon atoms, or a substituted or unsubstituted divalent heterocyclic group having 5 to 18 ring atoms.

In the organic EL device according to the exemplary embodiment, it is also preferable that mx is 1 or 2; and L₁₀₁ is a substituted or unsubstituted arylene group having 6 to 18 ring carbon atoms.

In the organic electroluminescence device according to the exemplary embodiment, it is preferable that at least one of R₁₀₁ to R₁₁₀ not being the bonding position to L₁₁₁ is a deuterium atom; and at least one of R₁₁₁ to R₁₂₀ not being the bonding position to L₁₁₂ is a deuterium atom.

In the organic EL device according to the exemplary embodiment, the compound represented by the formula (1) is also preferably represented by a formula (102) below.

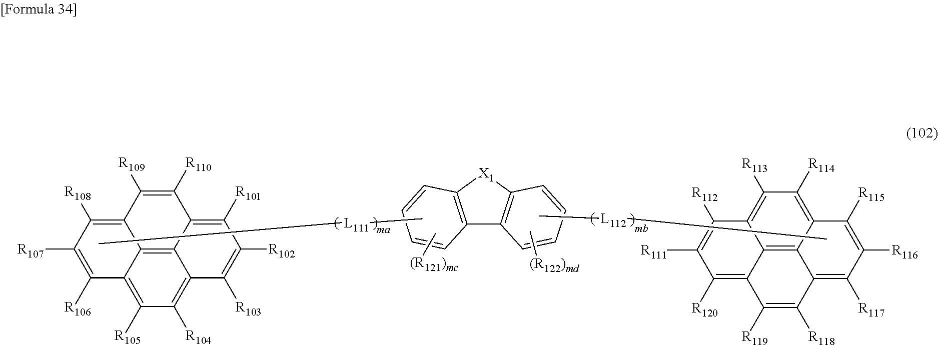

In the formula (102):
R₁₀₁ to R₁₂₀ each independently represent the same as R₁₀₁ to R₁₂₀ in the formula (101);
one of R₁₀₁ to R₁₁₀ represents a bonding position to L₁₁₁, and one of R₁₁₁ to R₁₂₀ represents a bonding position to L₁₁₂;
X₁ is CR₁₂₃R₁₂₄, an oxygen atom, a sulfur atom, or NR₁₂₅;
L₁₁₁ and L₁₁₂ are each independently a single bond, a substituted or unsubstituted arylene group having 6 to 50 ring carbon atoms, or a substituted or unsubstituted divalent heterocyclic group having 5 to 50 ring atoms;
ma is 0, 1, 2, 3, or 4;
mb is 0, 1, 2, 3, or 4;
ma+mb is 0, 1, 2, 3, or 4;
R₁₂₁, R₁₂₂, R₁₂₃, R₁₂₄ and R₁₂₅ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
mc is 3;
three R₁₂₁ are mutually the same or different;
md is 3; and
three R₁₂₂ are mutually the same or different.

In the compound represented by the formula (102), it is preferable that ma is 0, 1, or 2; and mb is 0, 1, or 2.

In the compound represented by the formula (102), it is preferable that ma is 0 or 1; and mb is 0 or 1.

In the compound represented by the formula (102), L₁₁₁ and L₁₁₂ are preferably each independently a single bond, a substituted or unsubstituted arylene group having 6 to 24 ring carbon atoms, or a substituted or unsubstituted divalent heterocyclic group having 5 to 24 ring atoms.

In the compound represented by the formula (102), ma is 1, 2, or 3; mb is 1, 2, or 3; and ma+mb is 2, 3, or 4.

In the compound represented by the formula (102), it is preferable that ma is 1 or 2; and mb is 1 or 2.

In the compound represented by the formula (102), it is preferable that ma is 1; and mb is 1.

In the organic EL device according to the exemplary embodiment, R₁₀₁ to R₁₁₀ not being the bonding position to L₁₁₁ are preferably each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the organic EL device according to the exemplary embodiment, R₁₀₁ to R₁₁₀ not being the bonding position to L₁₁₁ are preferably each independently a hydrogen atom, a substituted or unsubstituted alkyl group having 1 to 50 carbon atoms, or a substituted or unsubstituted cycloalkyl group having 3 to 50 ring carbon atoms.

In the organic EL device according to the exemplary embodiment, R₁₀₁ to R₁₁₀ not being the bonding position to L₁₁₁ are each preferably a hydrogen atom.

In the organic EL device according to the exemplary embodiment, R₁₁₁ to R₁₂₀ not being the bonding position to L₁₁₂ are preferably each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the organic EL device according to the exemplary embodiment, R₁₁₁ to R₁₂₀ not being the bonding position to L₁₁₂ are preferably each independently a hydrogen atom, a substituted or unsubstituted alkyl group having 1 to 50 carbon atoms, or a substituted or unsubstituted cycloalkyl group having 3 to 50 ring carbon atoms.

In the organic EL device according to the exemplary embodiment, R₁₁₁ to R₁₂₀ not being the bonding position to L₁₁₂ are each preferably a hydrogen atom.

In the organic EL device of the exemplary embodiment, two or more of R₁₀₁ to R₁₁₀ are each preferably a group represented by the formula (11).

In the organic EL device according to the exemplary embodiment, it is preferable that two or more of R₁₀₁ to R₁₁₀ are each a group represented by the formula (11); and Ar₁₀₁ is a substituted or unsubstituted aryl group having 6 to 50 ring carbon atoms.

In the organic EL device according to the exemplary embodiment, it is also preferable that:
Ar₁₀₁ is not a substituted or unsubstituted pyrenyl group;
L₁₀₁ is not a substituted or unsubstituted pyrenylene group; and
the substituted or unsubstituted aryl group having 6 to 50 ring carbon atoms as R₁₀₁ to R₁₁₀ not being the group represented by the formula (11) is not a substituted or unsubstituted pyrenyl group.

In the organic EL device according to the exemplary embodiment, it is preferable that R₁₀₁ to R₁₁₀ not being the group represented by the formula (11) are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the organic EL device according to the exemplary embodiment, it is preferable that R₁₀₁ to R₁₁₀ not being the group represented by the formula (11) are each independently a hydrogen atom, a substituted or unsubstituted alkyl group having 1 to 50 carbon atoms, or a substituted or unsubstituted cycloalkyl group having 3 to 50 ring carbon atoms.

In the organic EL device according to the exemplary embodiment, R₁₀₁ to R₁₁₀ not being the group represented by the formula (11) are each preferably a hydrogen atom.

In the organic EL device according to the exemplary embodiment, for instance, two of R₁₀₁ to R₁₁₀ in the compound represented by the formula (1) are each a group represented by the formula (11).

In the organic EL device according to the exemplary embodiment, for instance, three of R₁₀₁ to R₁₁₀ in the compound represented by the formula (1) are each a group represented by the formula (11).

In the organic EL device according to the exemplary embodiment, for instance, four of R₁₀₁ to R₁₁₀ in the compound represented by the formula (1) are each a group represented by the formula (11).

In the organic EL device according to the exemplary embodiment, for instance, one of R₁₀₁ to R₁₁₀ in the compound represented by the formula (1) is a group represented by the formula (11), and mx is 1 or more.

In the organic EL device according to the exemplary embodiment, for instance, one of R₁₀₁ to R₁₁₀ in the compound represented by the formula (1) is a group represented by the formula (11), mx is 0, and Ar₁₀₁ is a substituted or unsubstituted aryl group.

In the organic EL device according to the exemplary embodiment, for instance, one of R₁₀₁ to R₁₁₀ in the compound represented by the formula (1) is a group represented by the formula (11), mx is 0, and Ar₁₀₁ is a substituted or unsubstituted heterocyclic group including a nitrogen atom.

In the organic EL device according to the exemplary embodiment, for instance, one of R₁₀₁ to R₁₁₀ in the compound represented by the formula (1) is a group represented by the formula (11), mx is 0, and Ar₁₀₁ is a substituted or unsubstituted heterocyclic group including a sulfur atom.

In the organic EL device according to the exemplary embodiment, for instance, one of R₁₀₁ to R₁₁₀ in the compound represented by the formula (1) is a group represented by the formula (11), mx is 0, and Ar₁₀₁ is a substituted or unsubstituted furyl group, oxazolyl group, isoxazolyl group, oxadiazolyl group, xanthenyl group, benzofuranyl group, isobenzofuranyl group, dibenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

In the organic EL device according to the exemplary embodiment, for instance, one of R₁₀₁ to R₁₁₀ in the compound represented by the formula (1) is a group represented by the formula (11), mx is 0, and Ar₁₀₁ is at least one group selected from the group consisting of unsubstituted furyl group, oxazolyl group, isoxazolyl group, oxadiazolyl group, xanthenyl group, benzofuranyl group, isobenzofuranyl group, dibenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

In the organic EL device according to the exemplary embodiment, for instance, one of R₁₀₁ to R₁₁₀ in the compound represented by the formula (1) is a group represented by the formula (11), mx is 0, and Ar₁₀₁ is a substituted or unsubstituted dibenzofuranyl group.

In the organic EL device according to the exemplary embodiment, for instance, one of R₁₀₁ to R₁₁₀ in the compound represented by the formula (1) is a group represented by the formula (11), mx is 0, and Ar₁₀₁ is an unsubstituted dibenzofuranyl group.

In the organic EL device according to the exemplary embodiment, for instance, mx in the compound represented by the formula (101) is 2 or more.

In the organic EL device according to the exemplary embodiment, for instance, mx in the compound represented by the formula (101) is 1 or more, and L₁₀₁ is an arylene group having 6 to 24 ring carbon atoms or a divalent heterocyclic group having 5 to 24 ring atoms.

In the organic EL device according to the exemplary embodiment, for instance, mx in the compound represented by the formula (101) is 1 or more, and L₁₀₁ is an arylene group having 6 to 18 ring carbon atoms or a divalent heterocyclic group having 5 to 18 ring atoms.

### Method of Manufacturing Compound Represented by Formula (1)

The compound represented by the formula (1) can be manufactured by a known method. The compound represented by the formula (1) can also be manufactured based on a known method through a known alternative reaction using a known material(s) tailored for the target compound.

### Specific Examples of Compound Represented by Formula (1)

Specific examples of the compound represented by the formula (1) include the following compounds. It should however be noted that the invention is not limited to the specific examples.

In the specific examples of the compound herein, D represents a deuterium atom, Me represents a methyl group, and tBu represents a tert-butyl group.

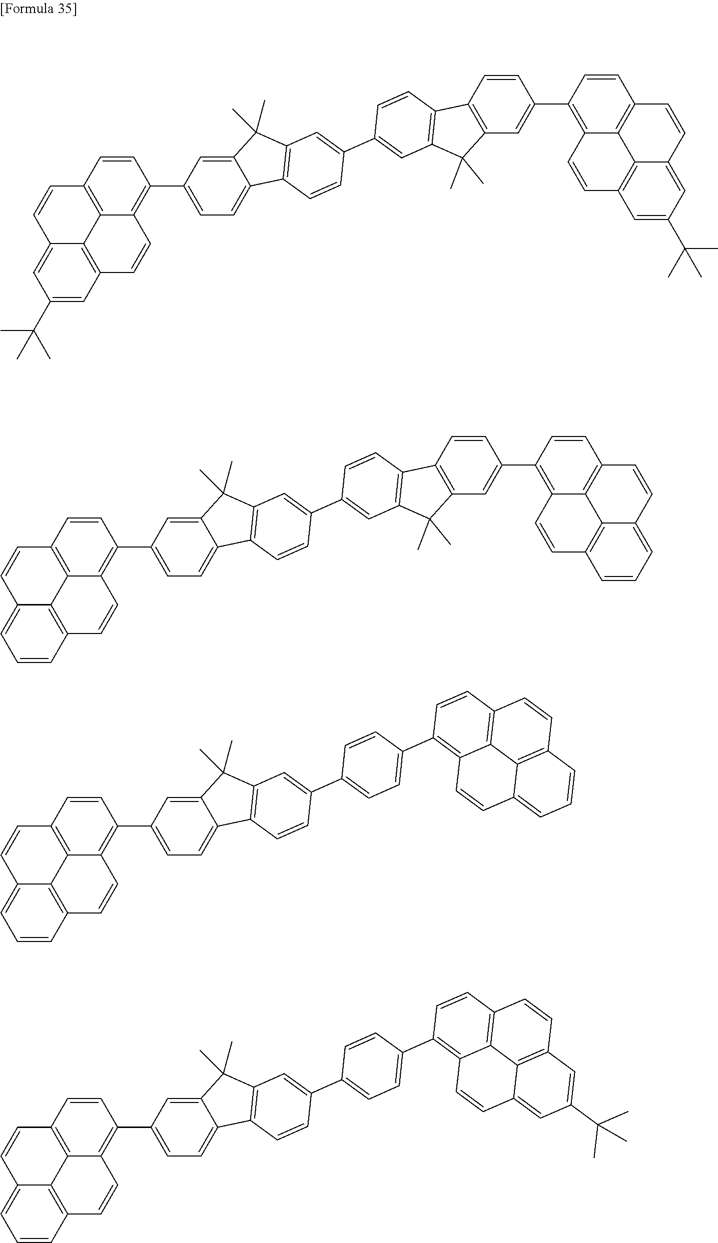

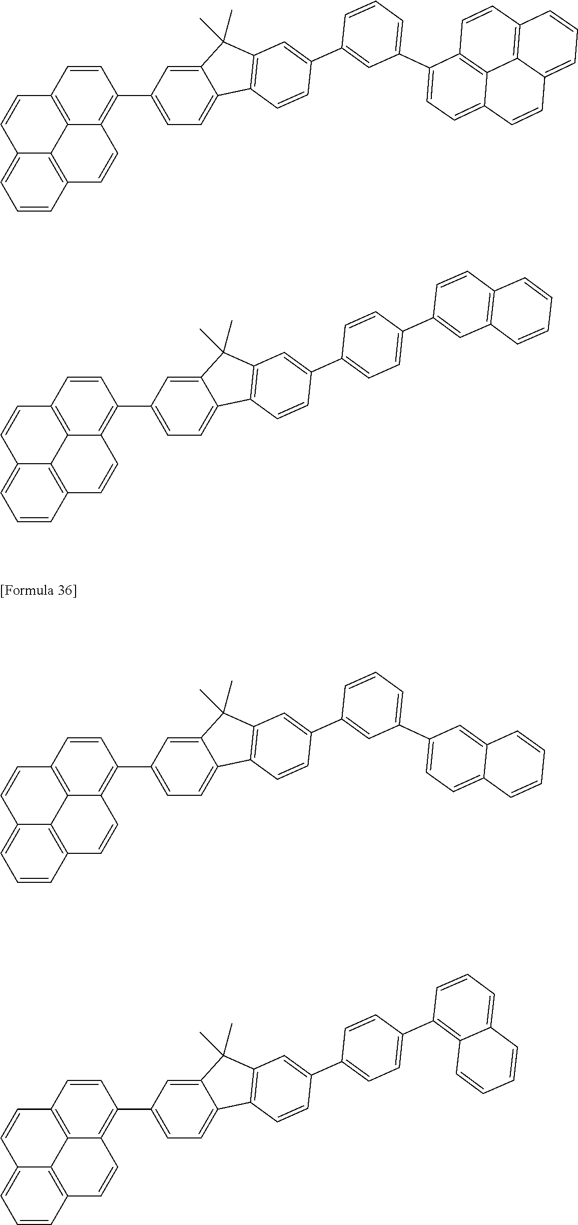

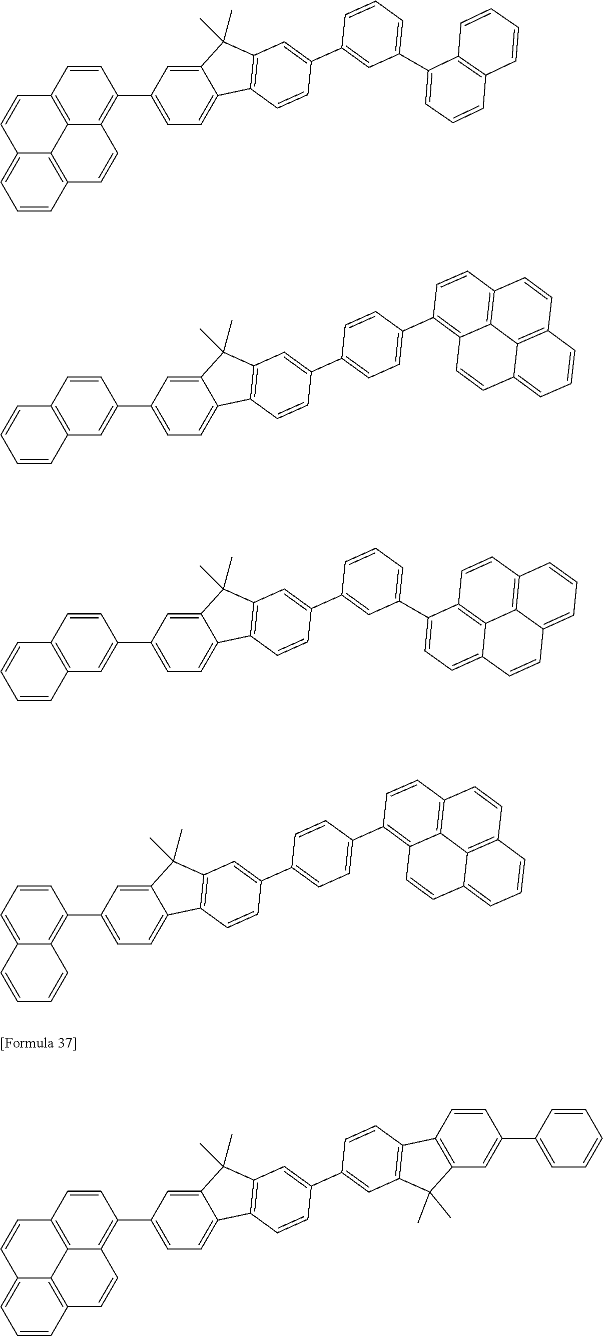

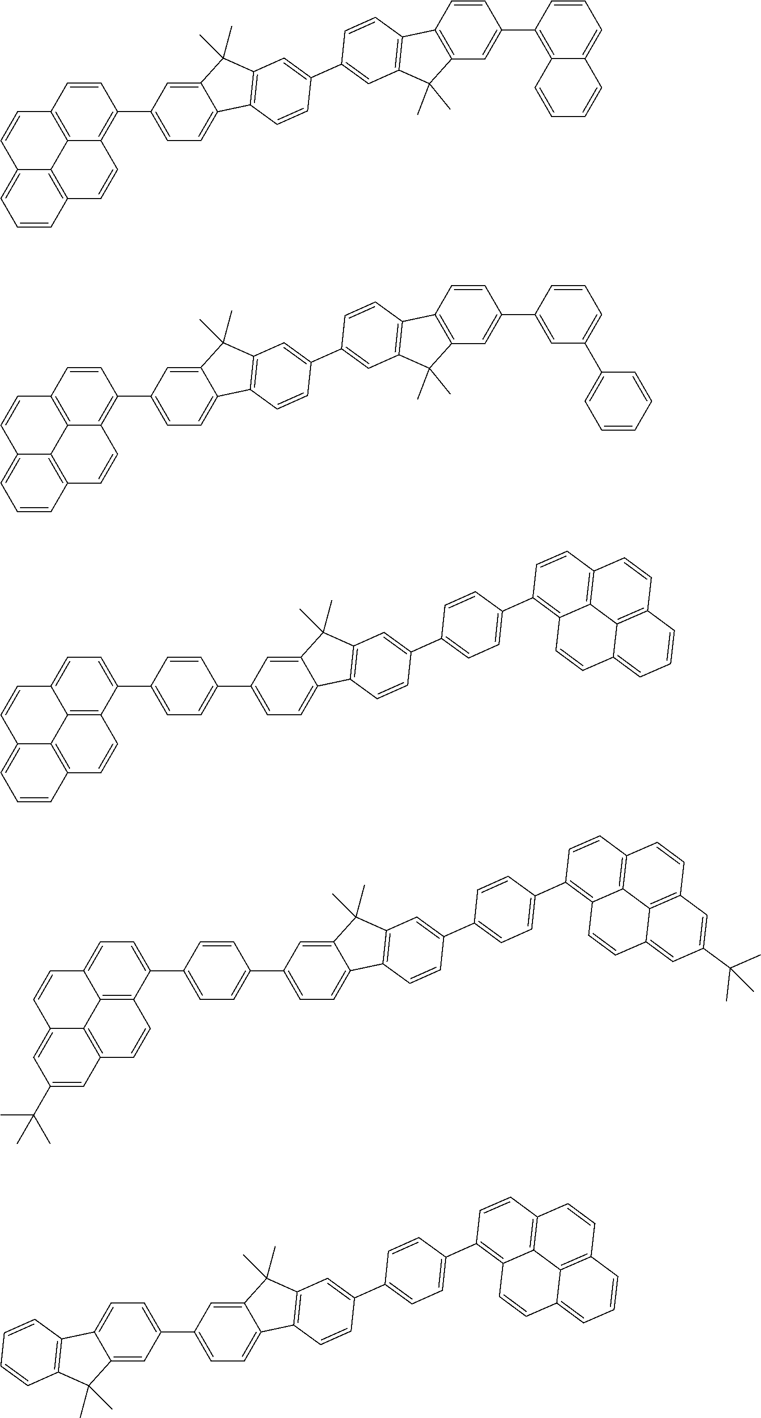

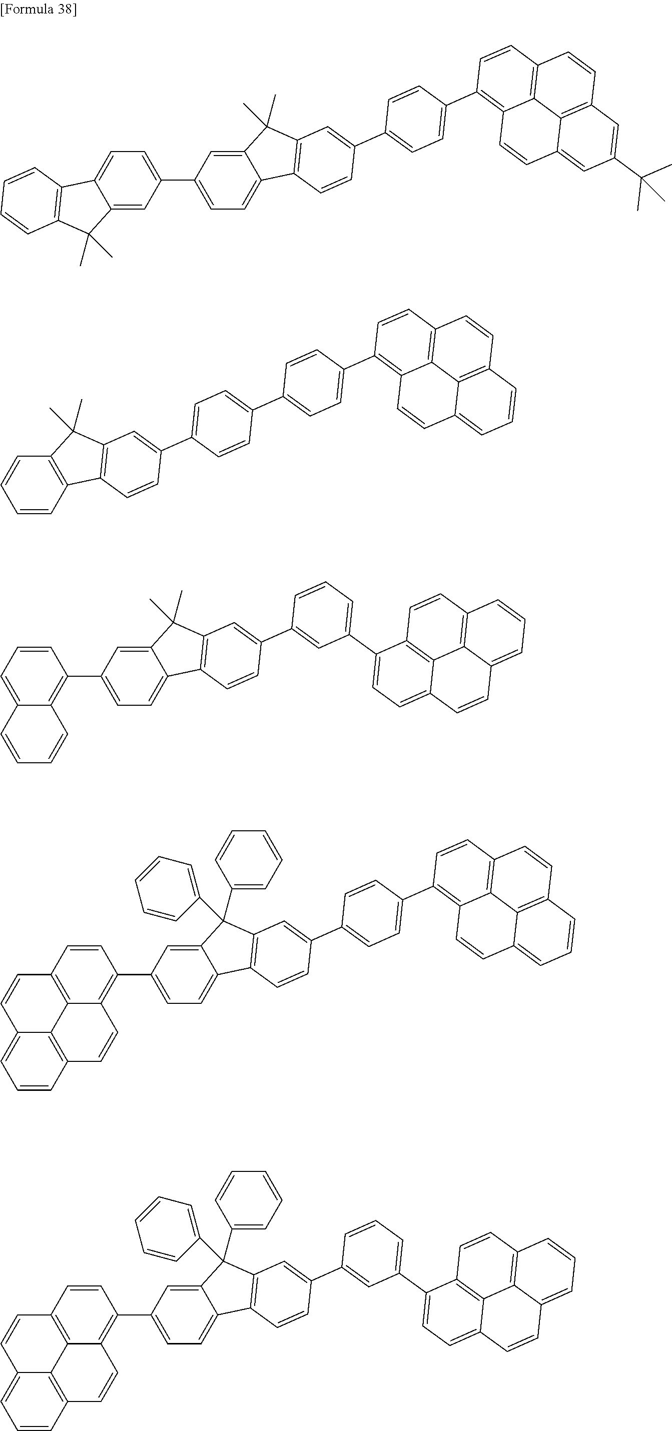

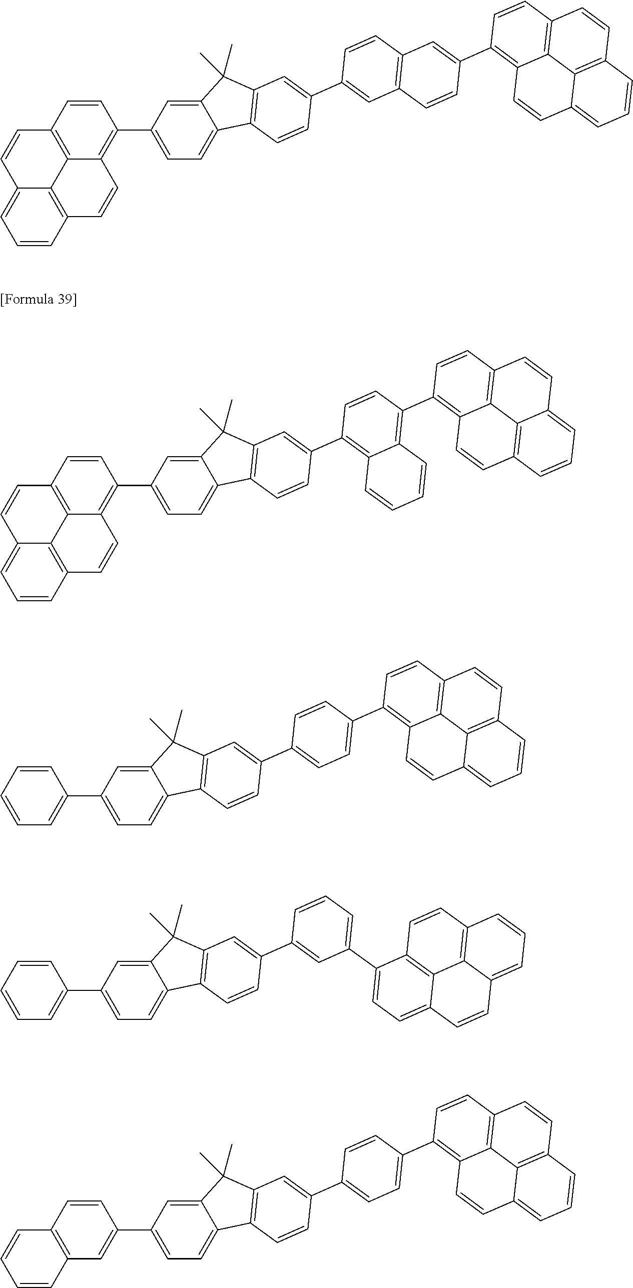

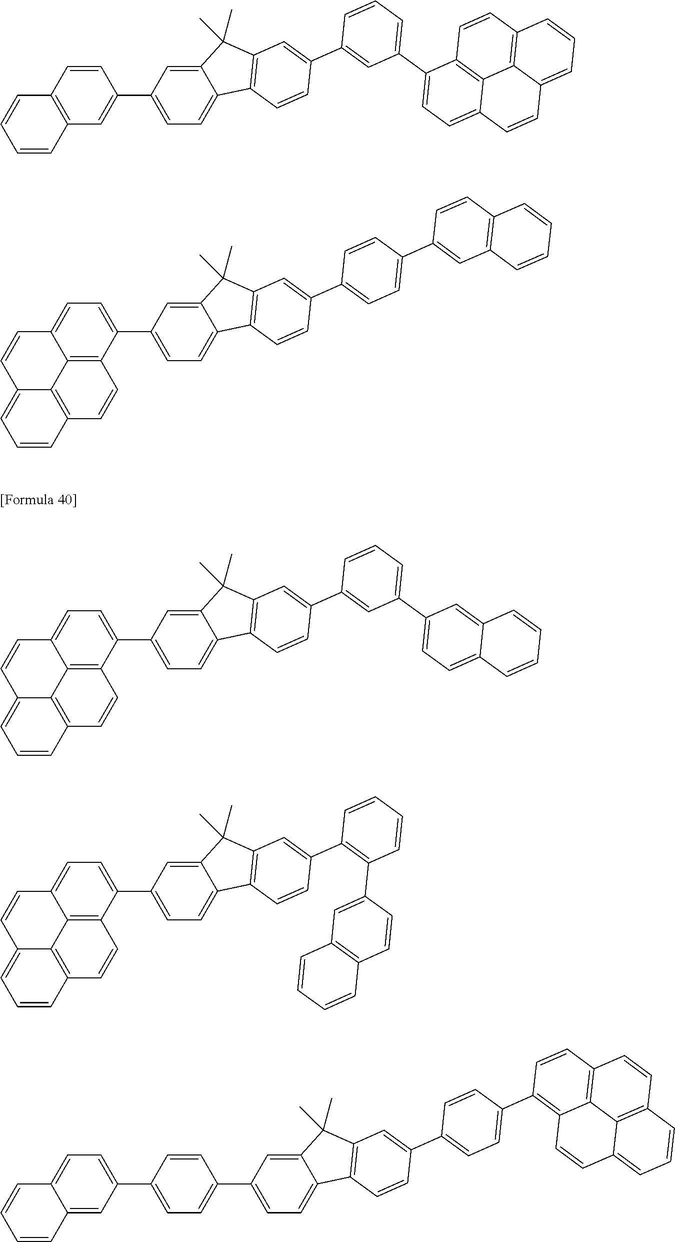

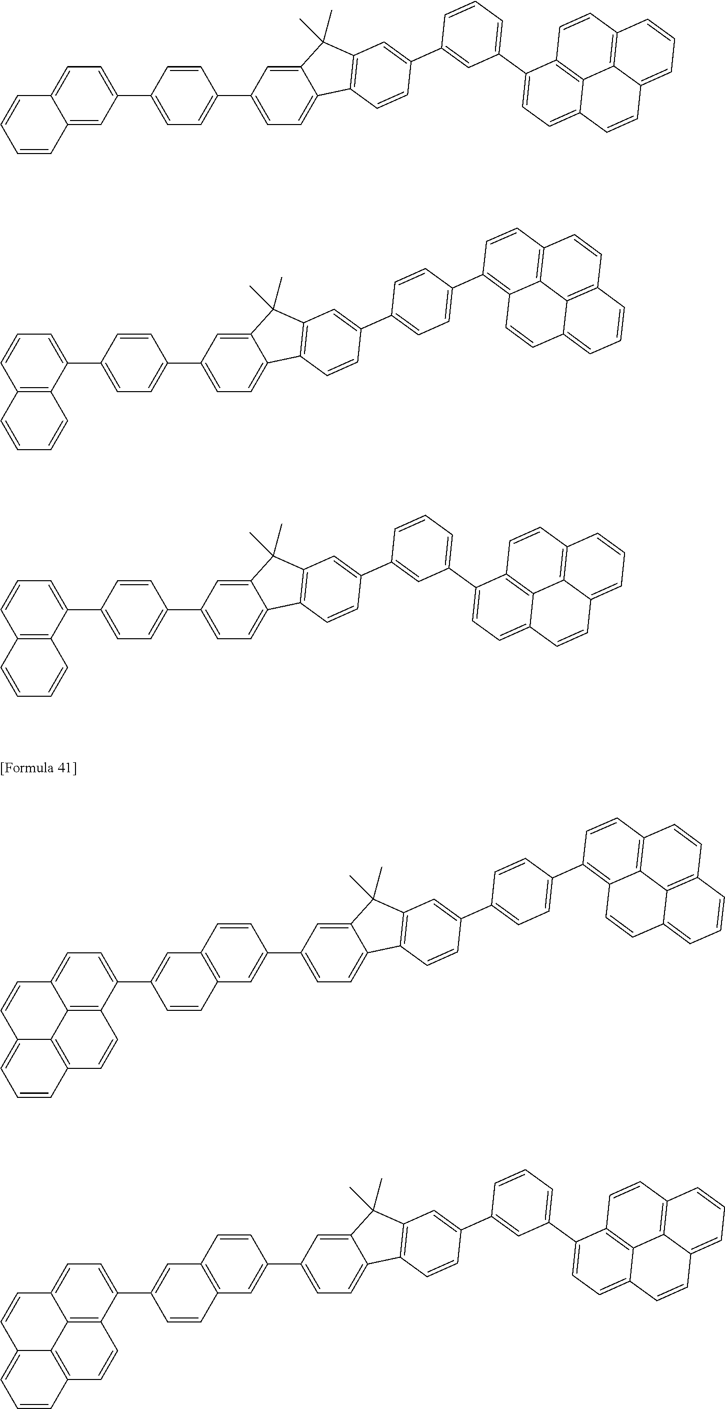

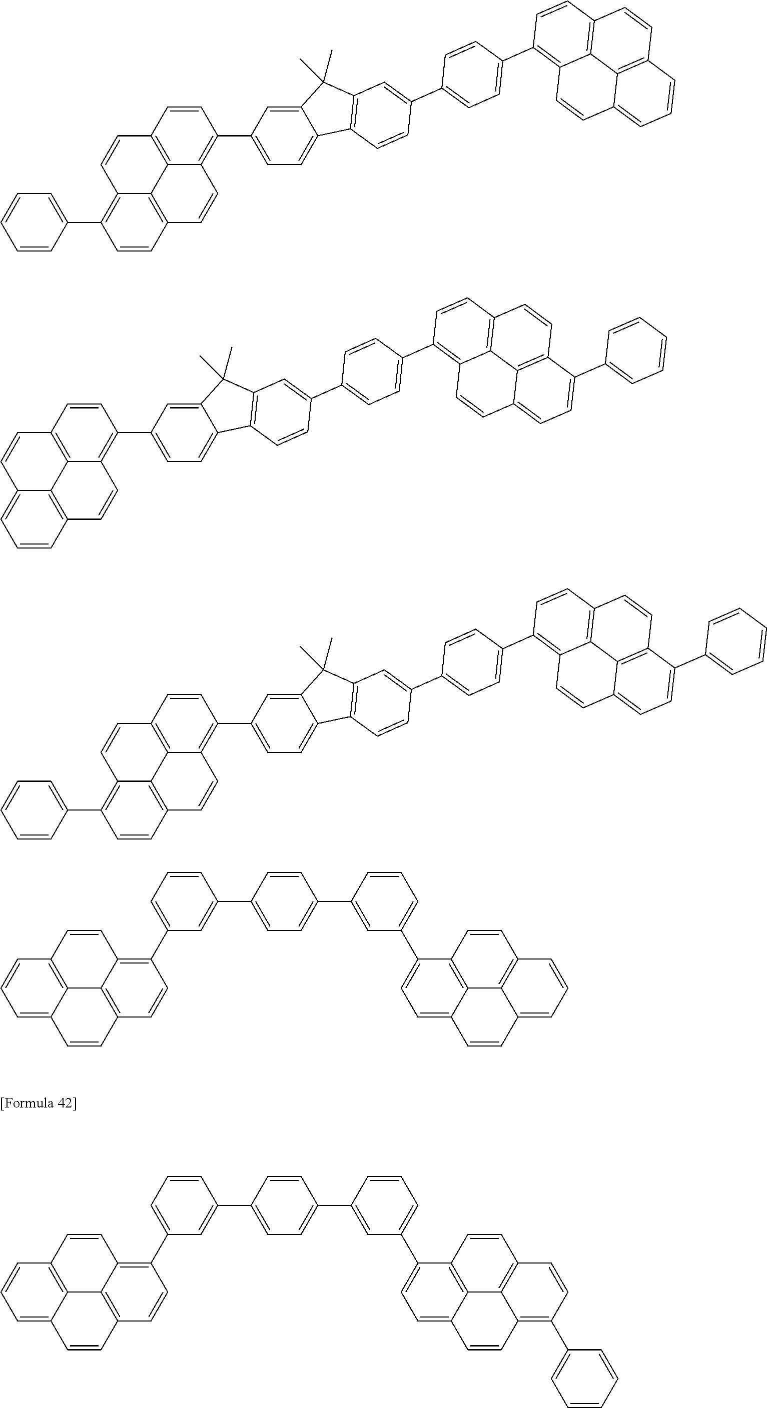

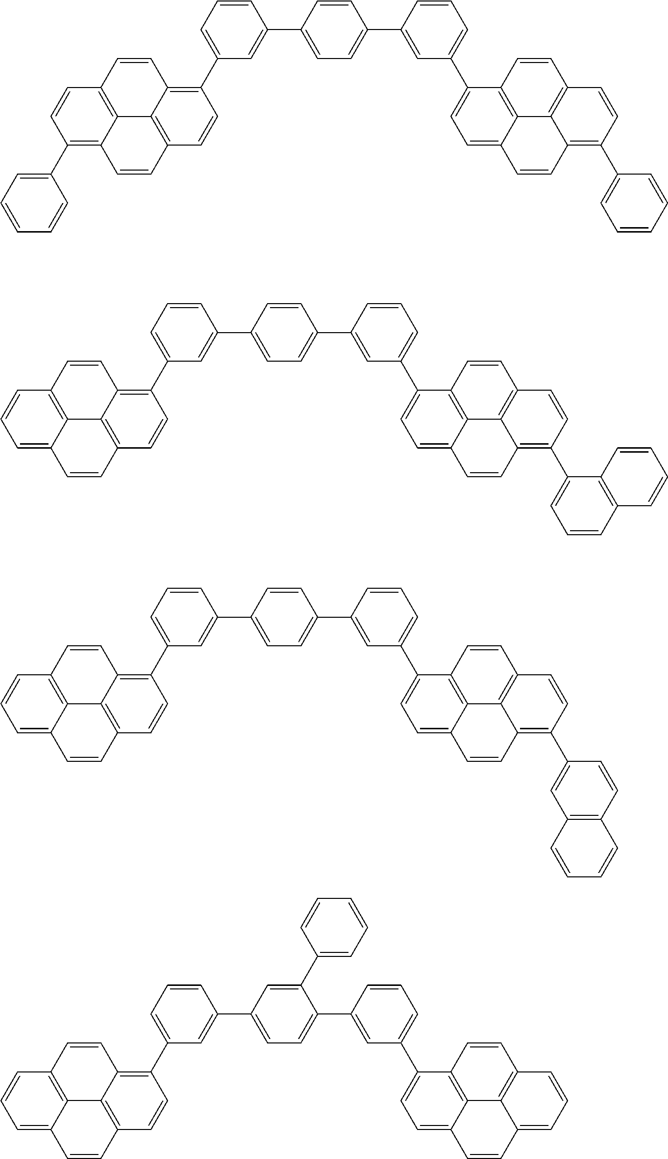

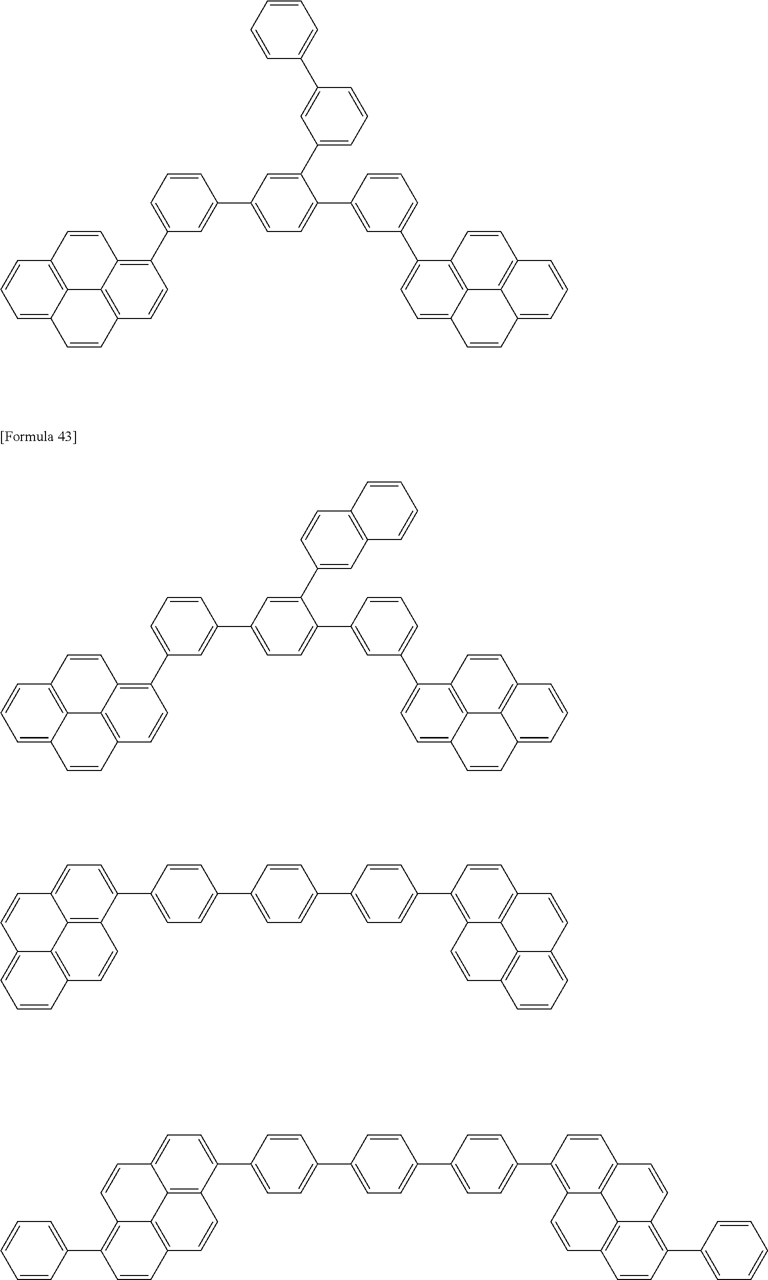

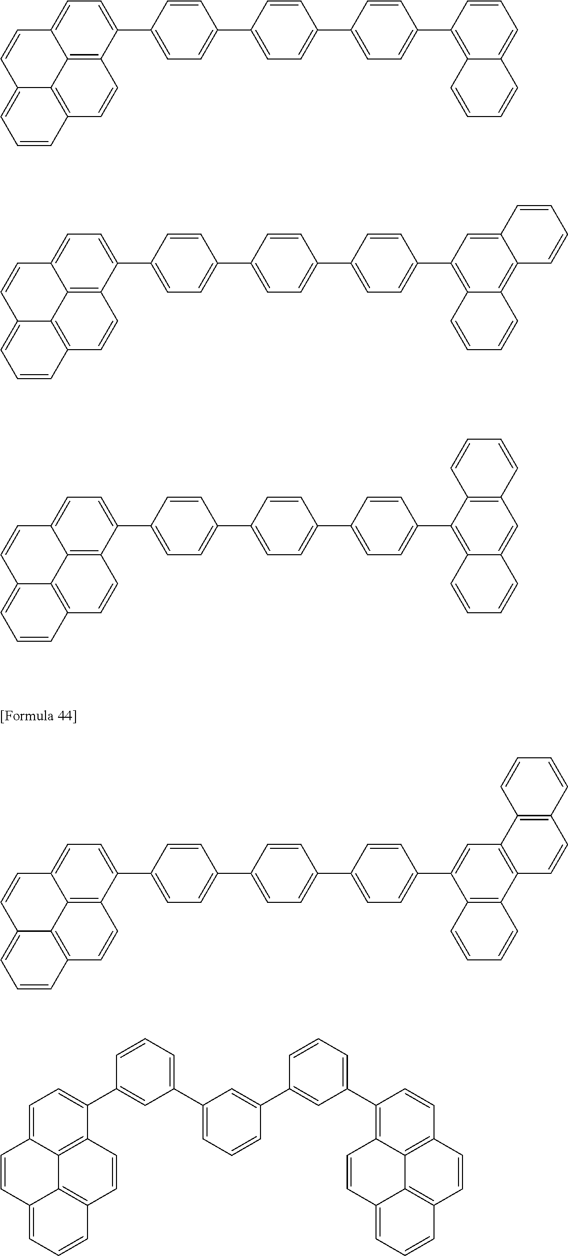

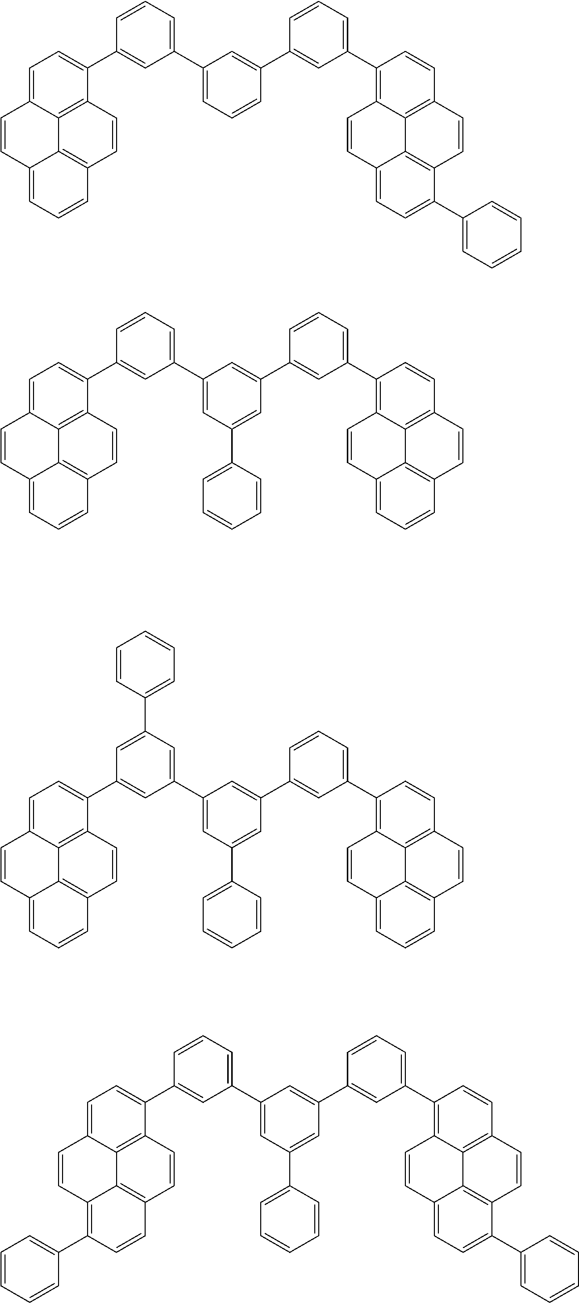

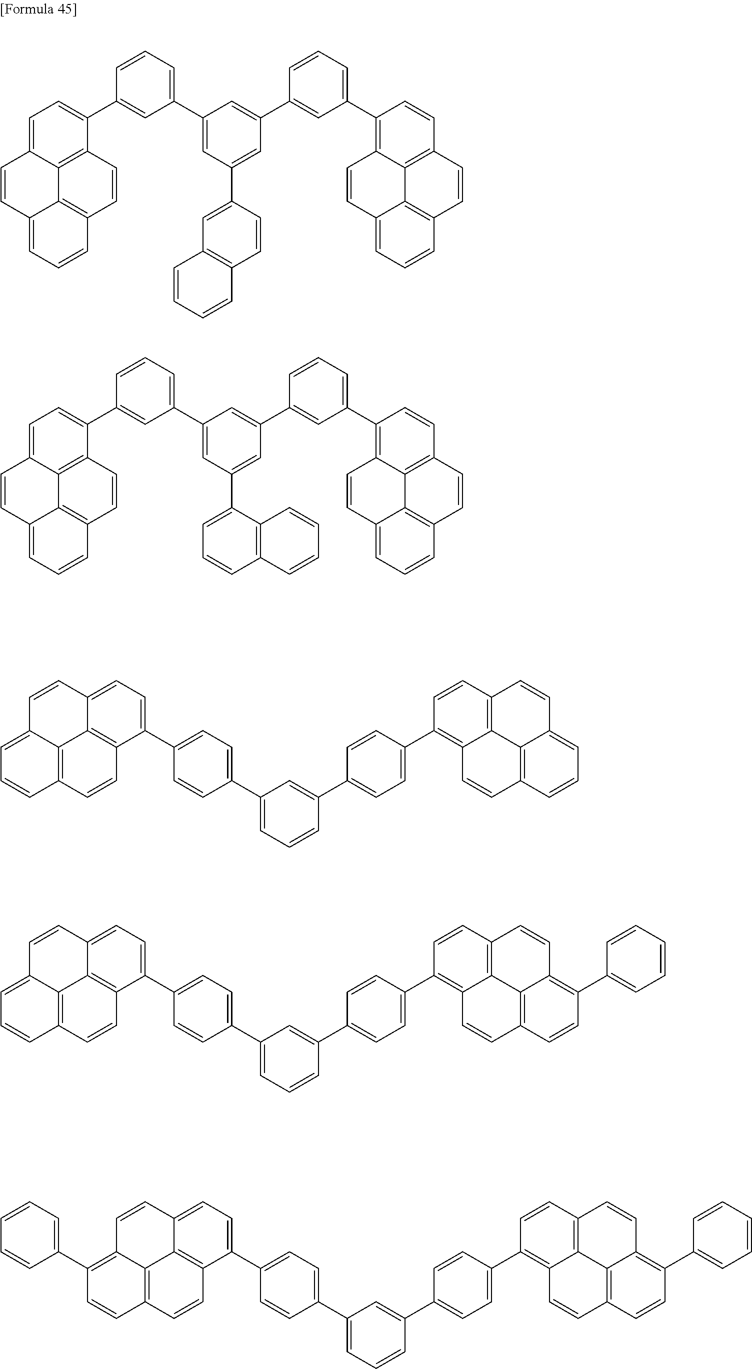

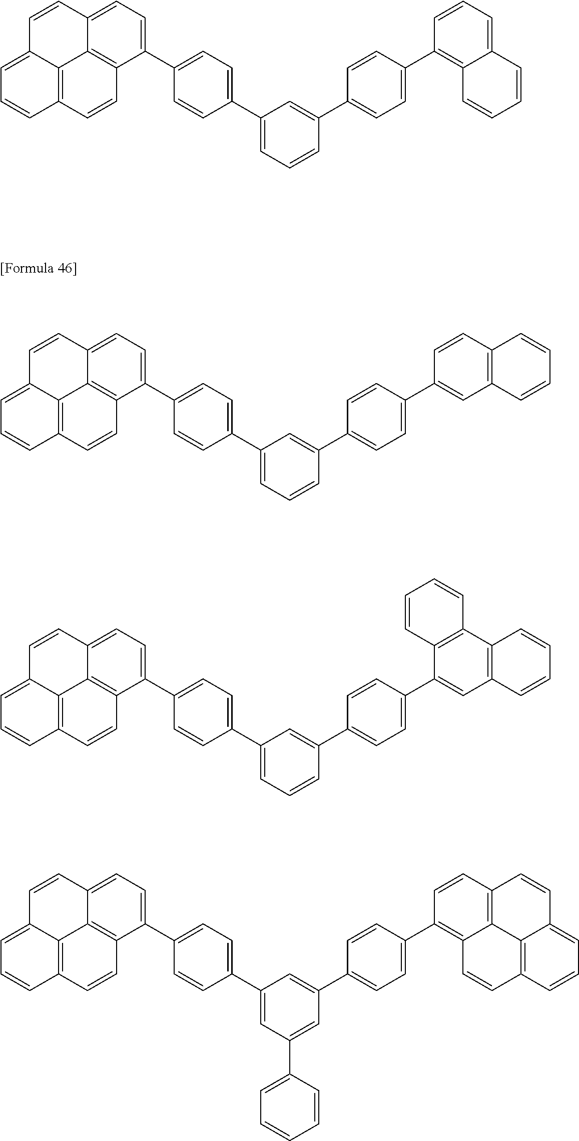

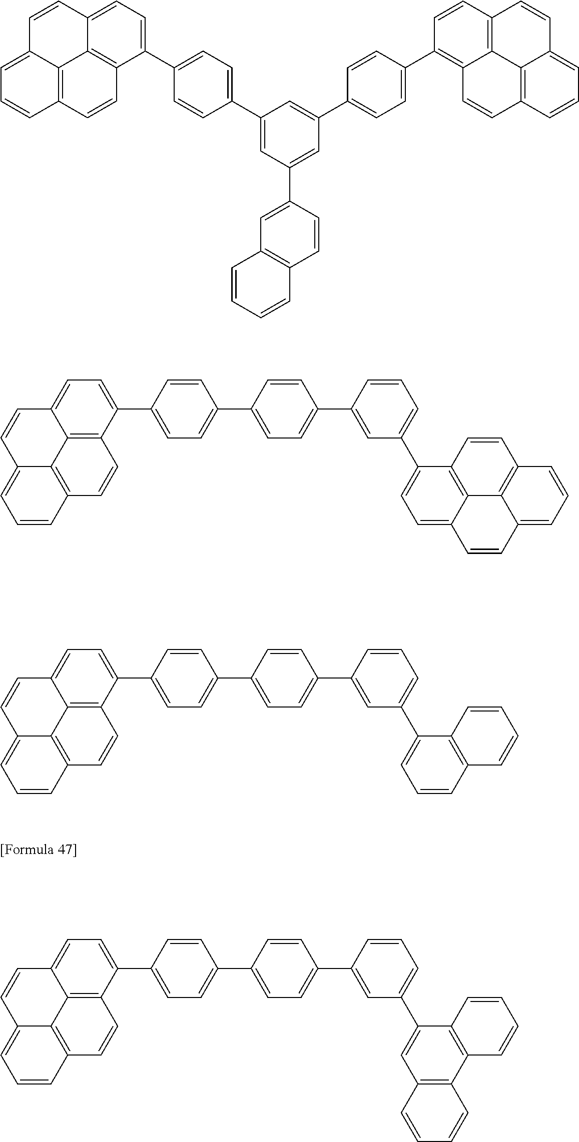

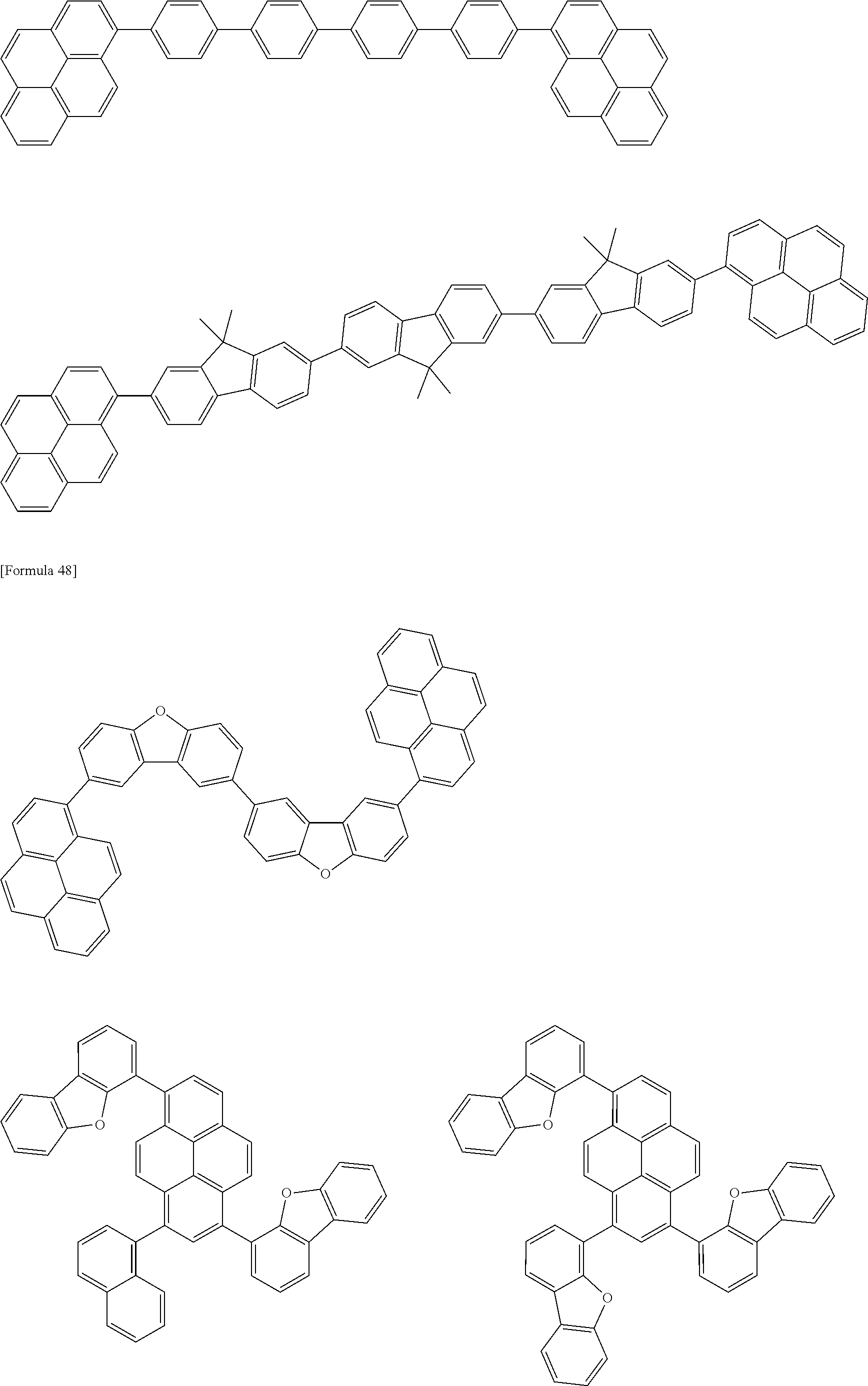

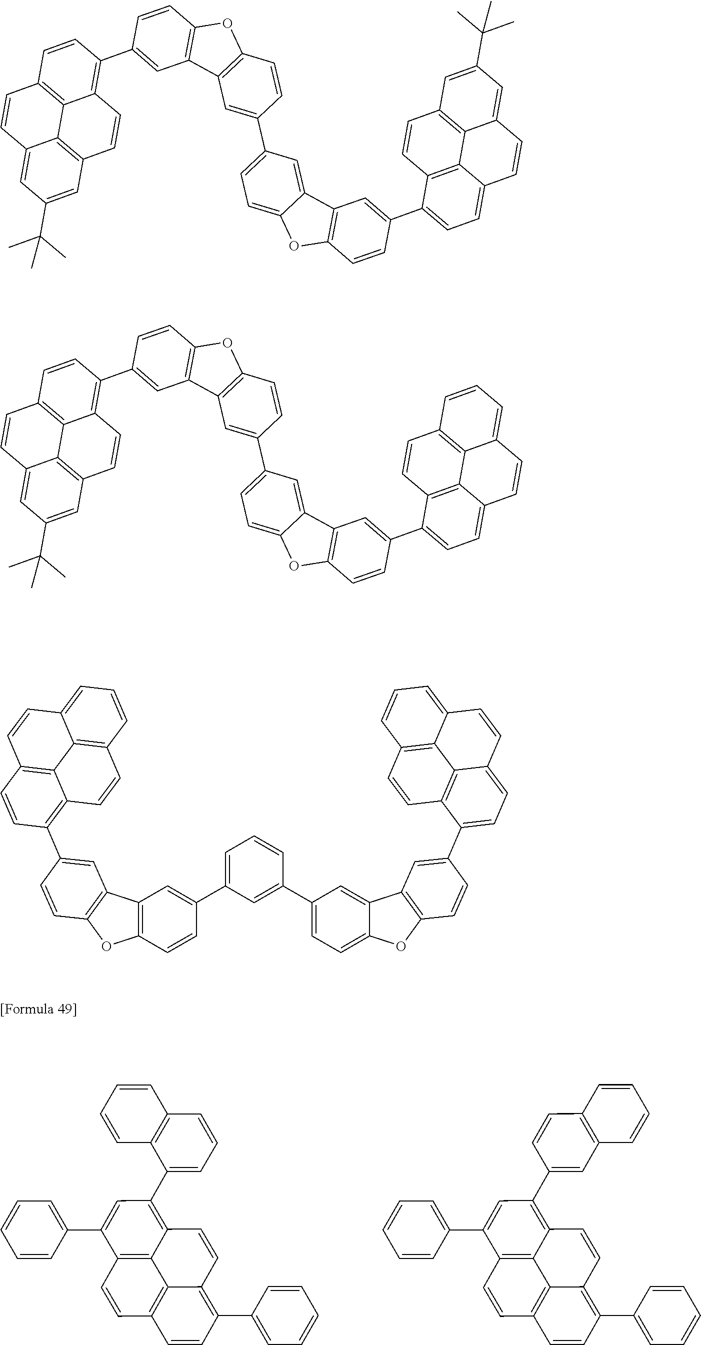

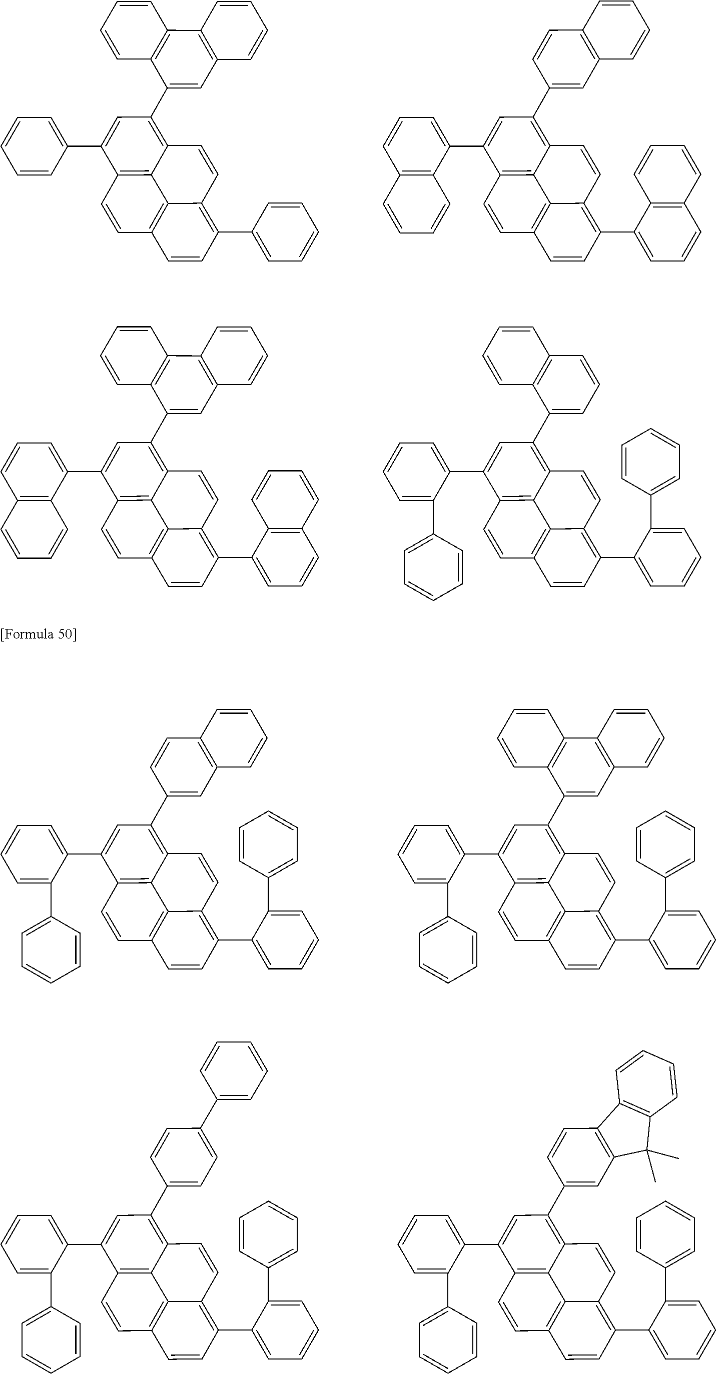

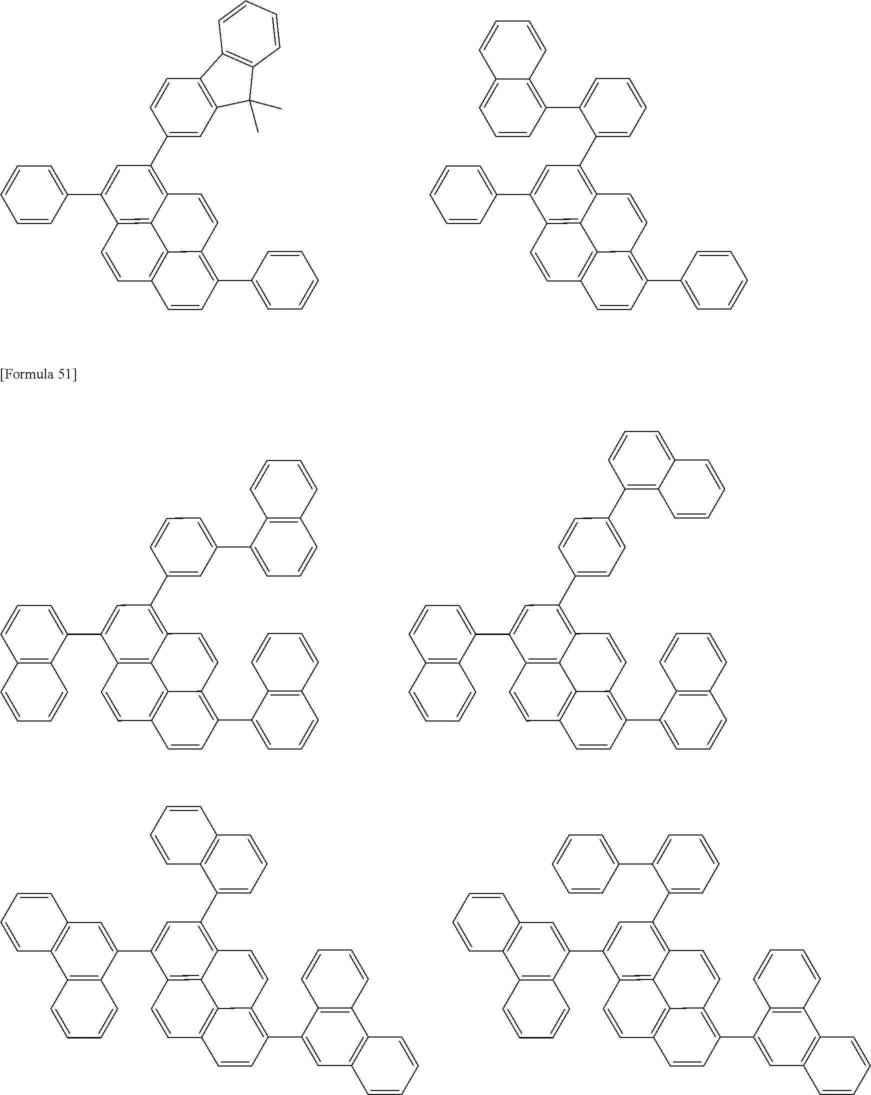

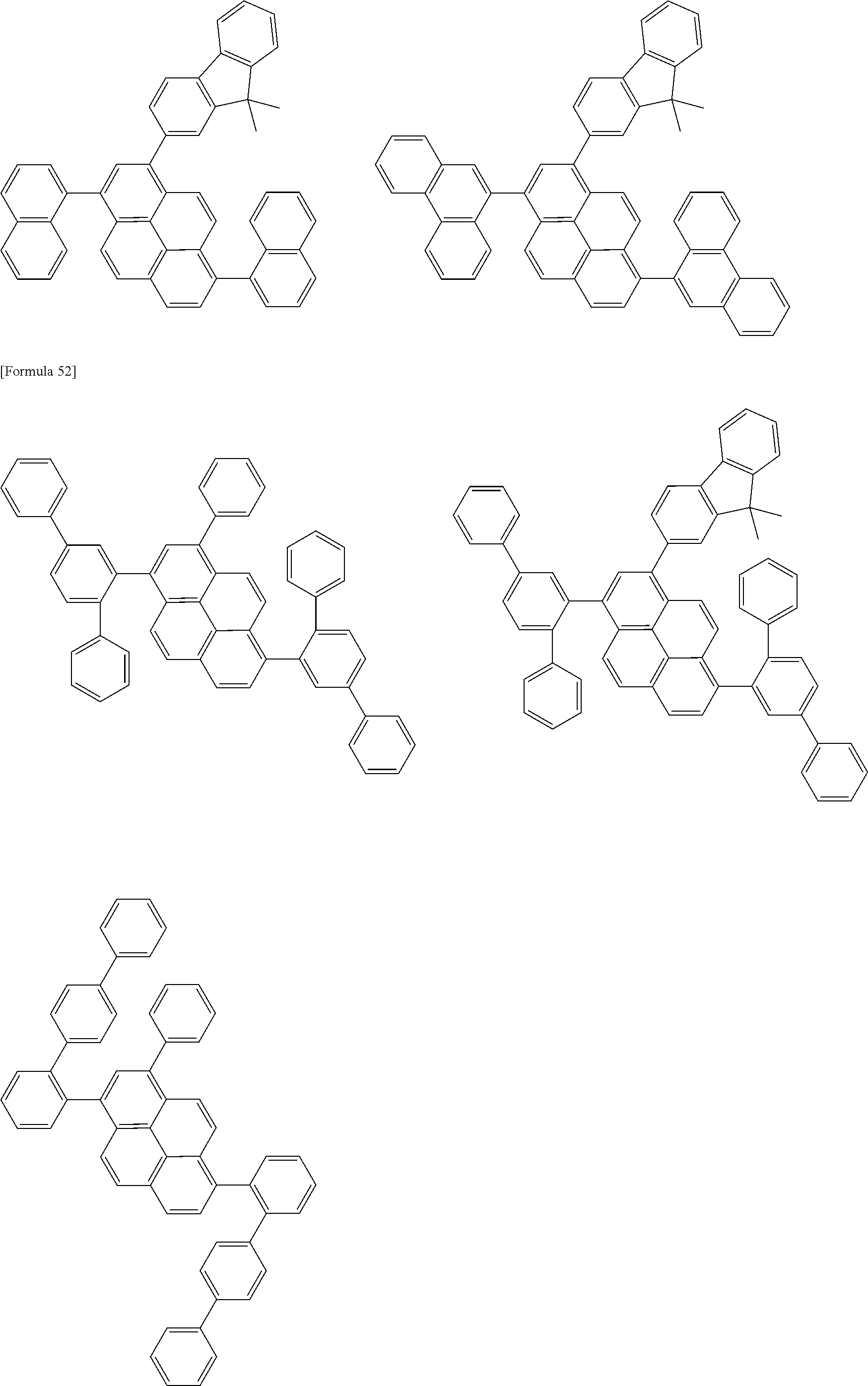

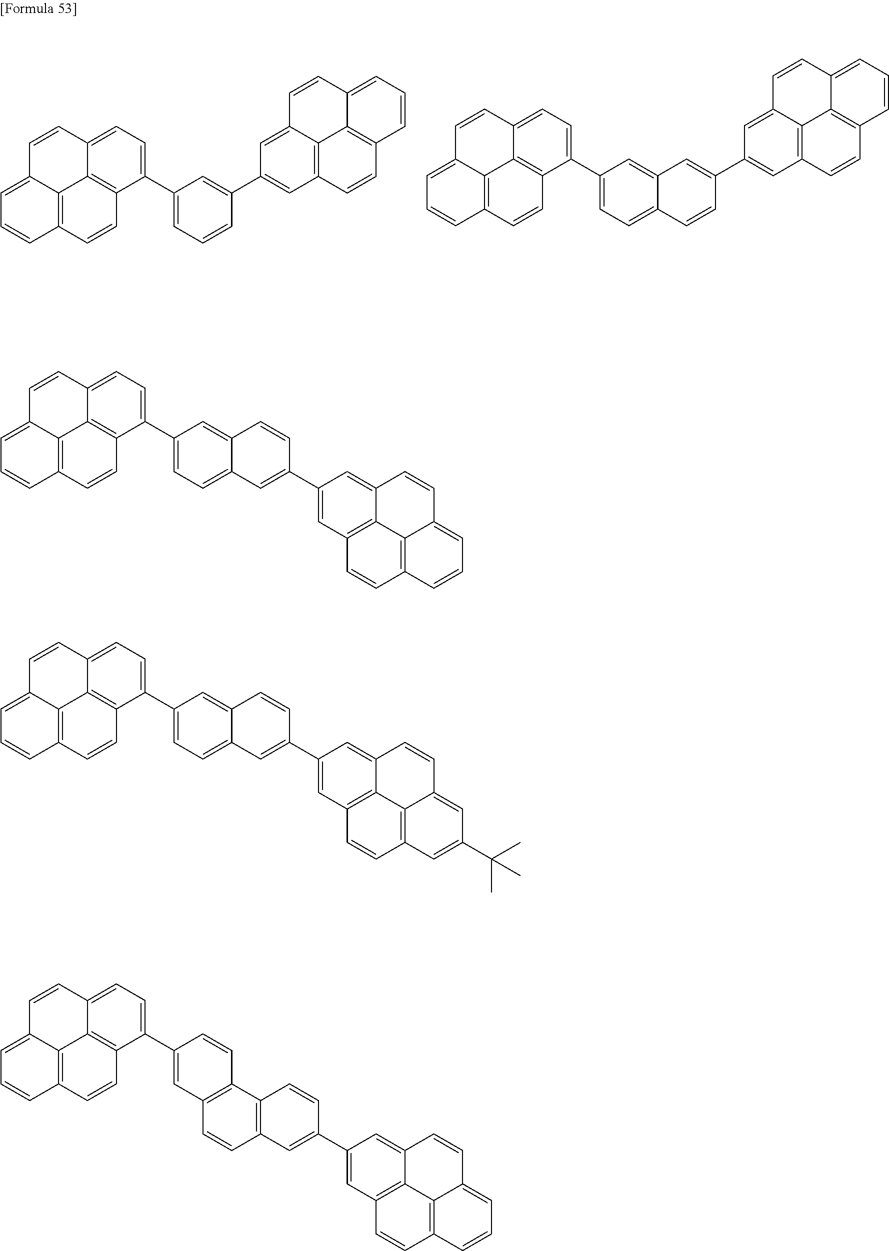

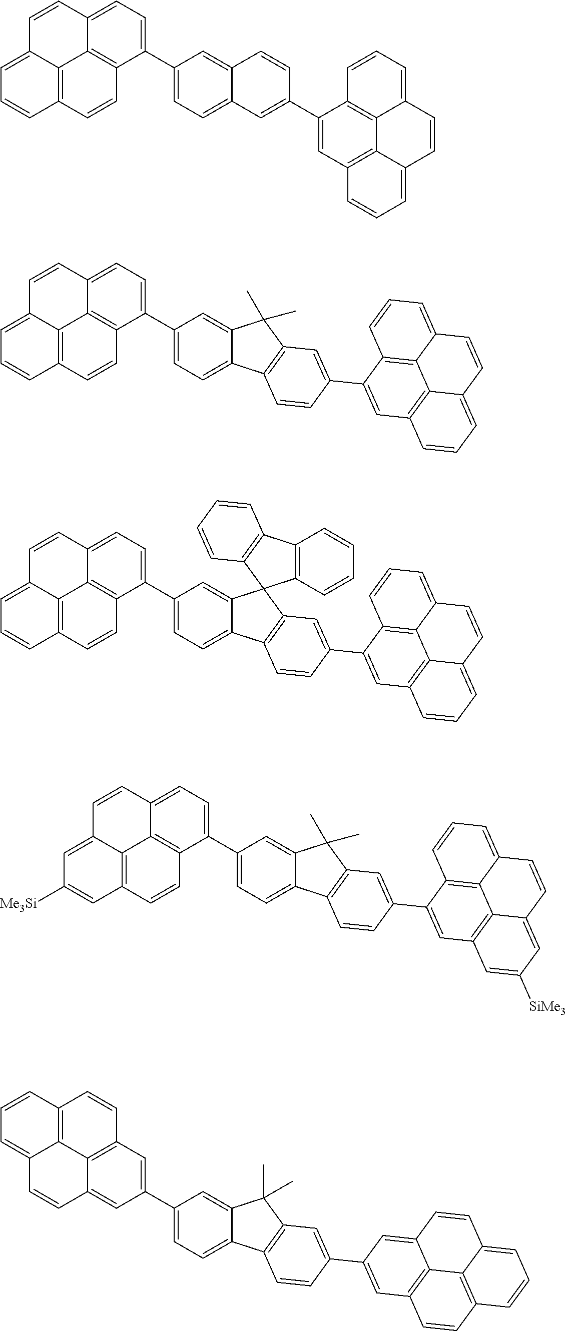

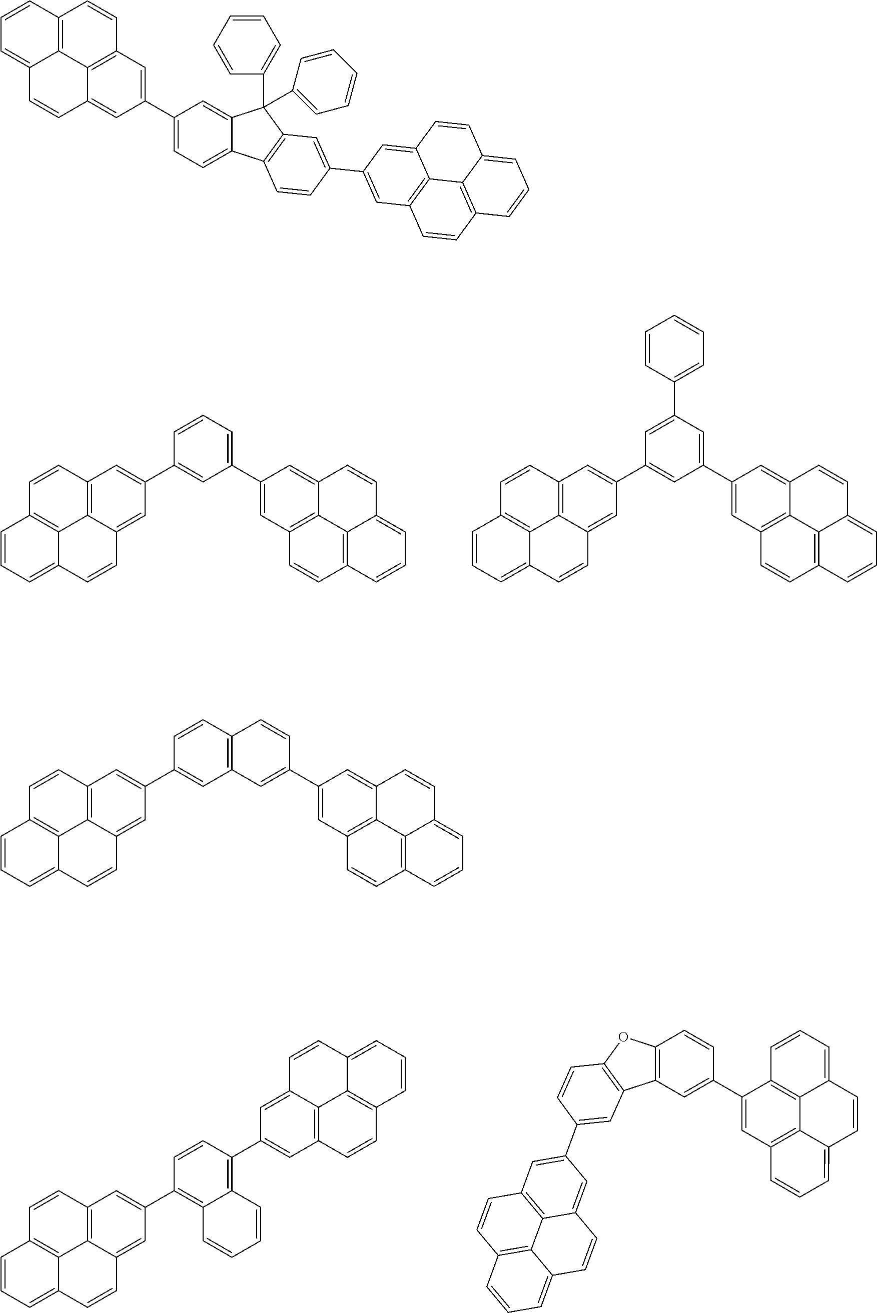

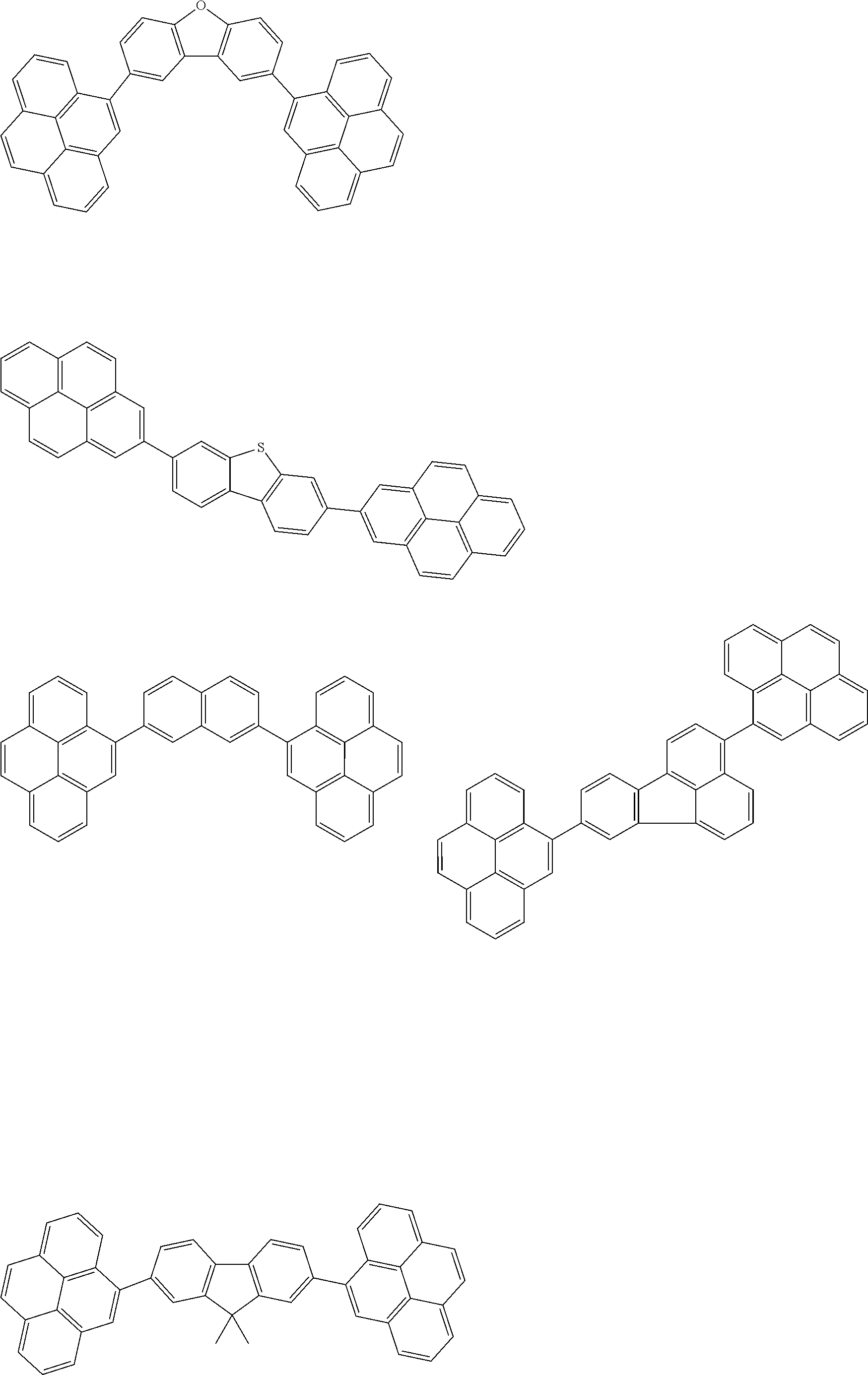

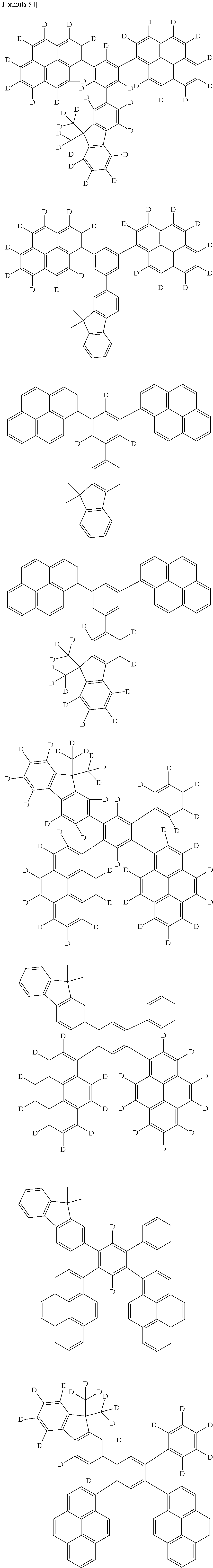

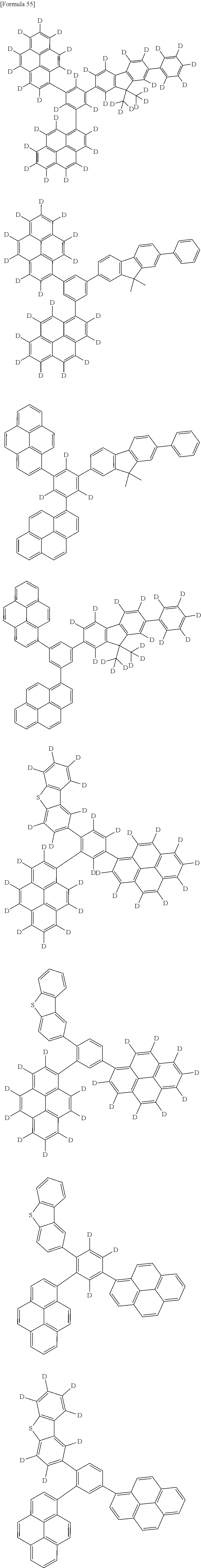

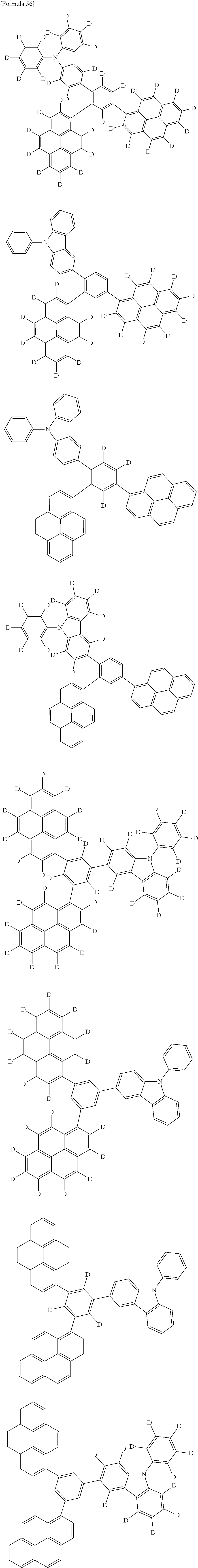

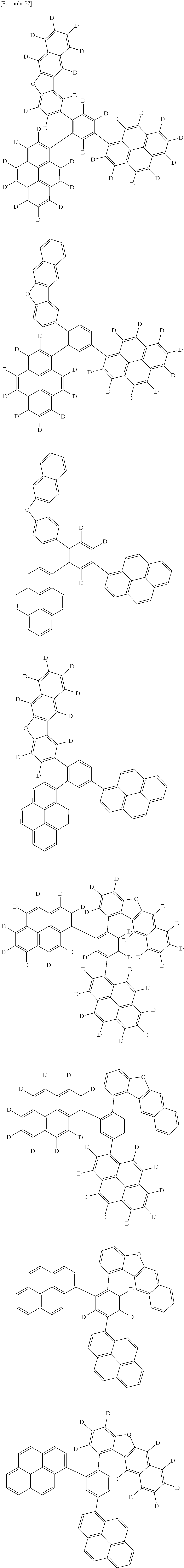

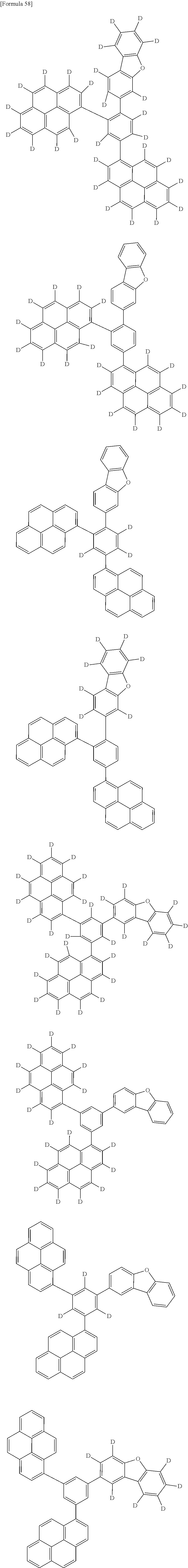

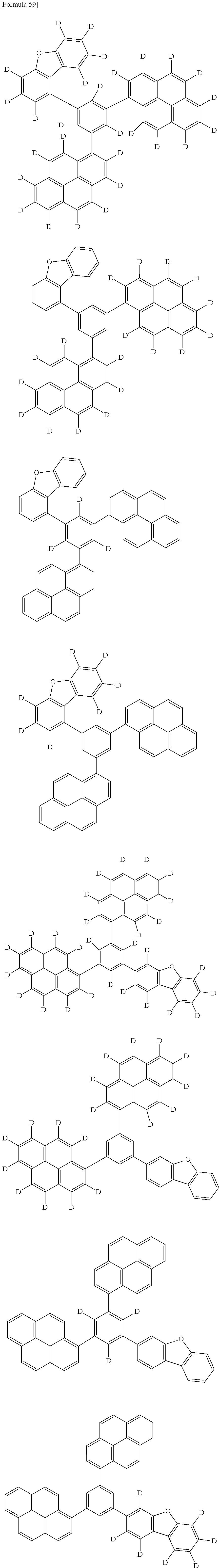

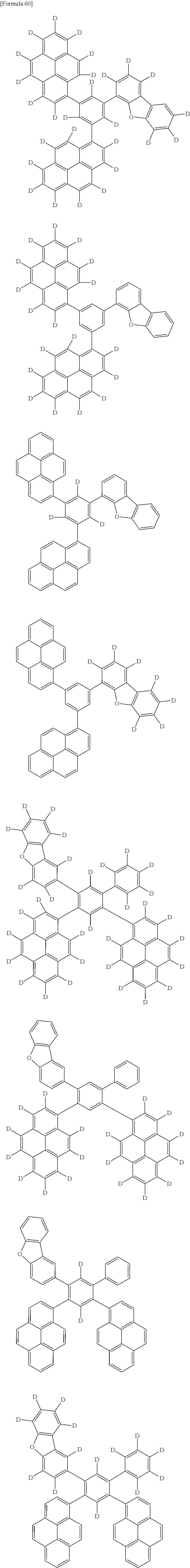

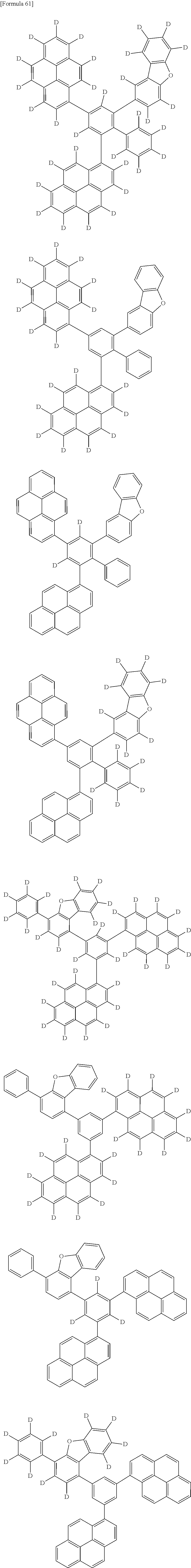

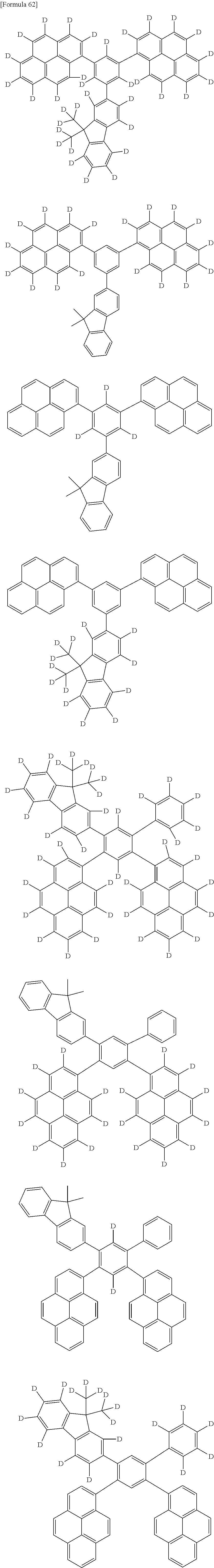

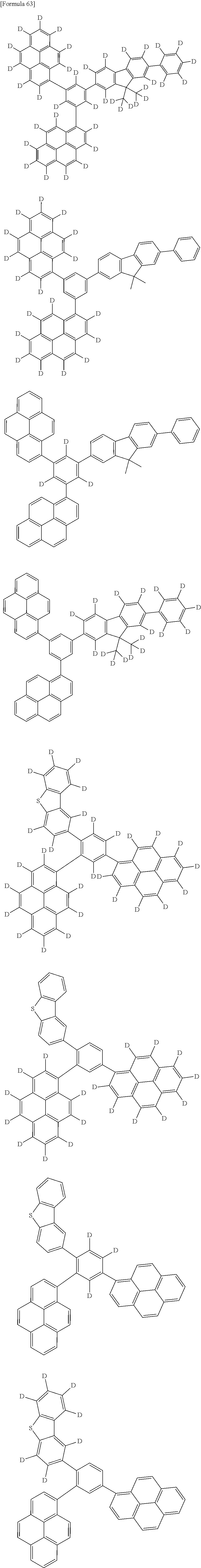

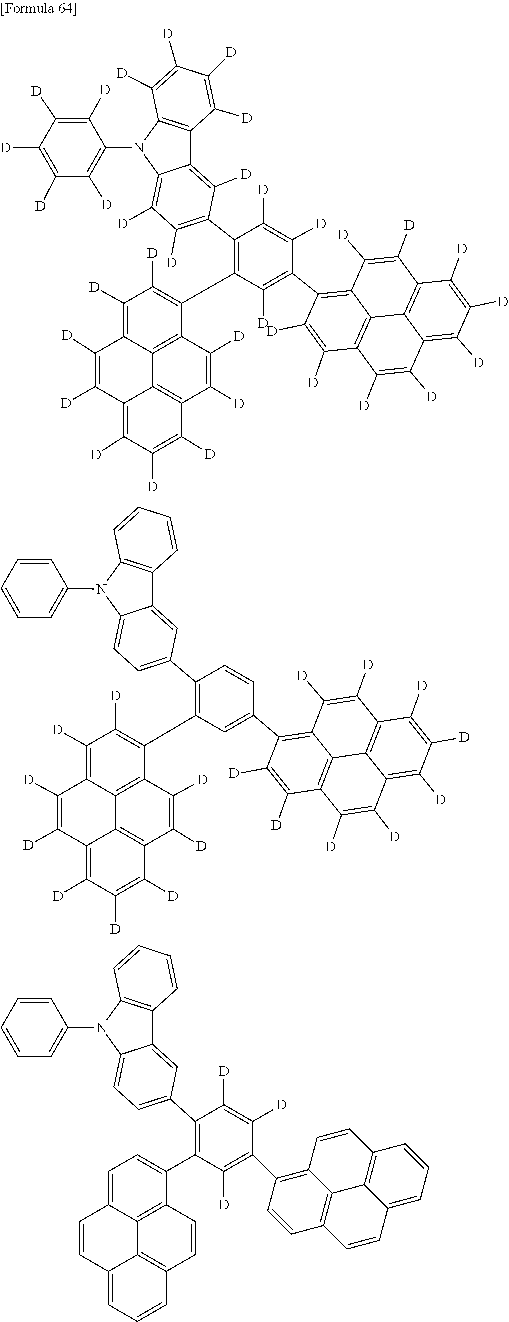

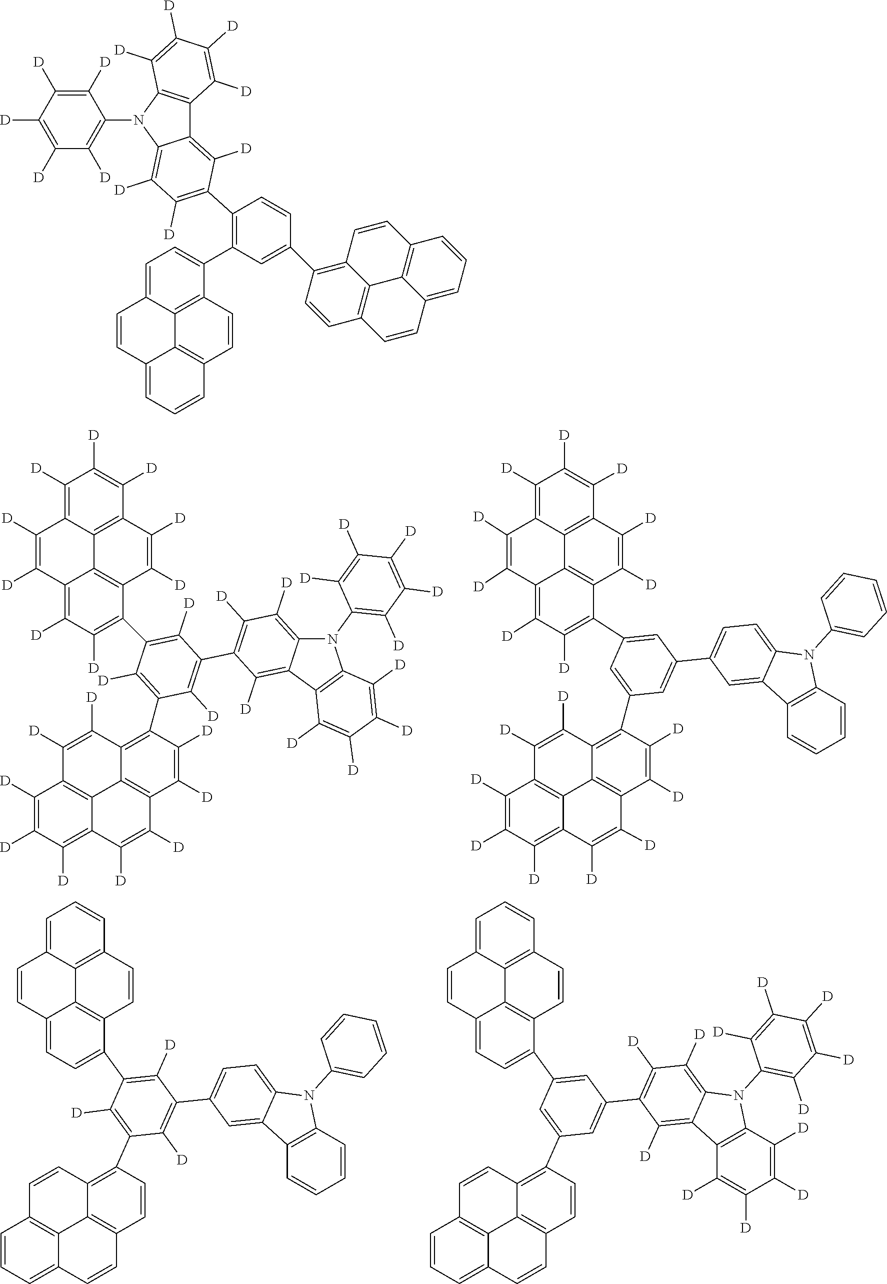

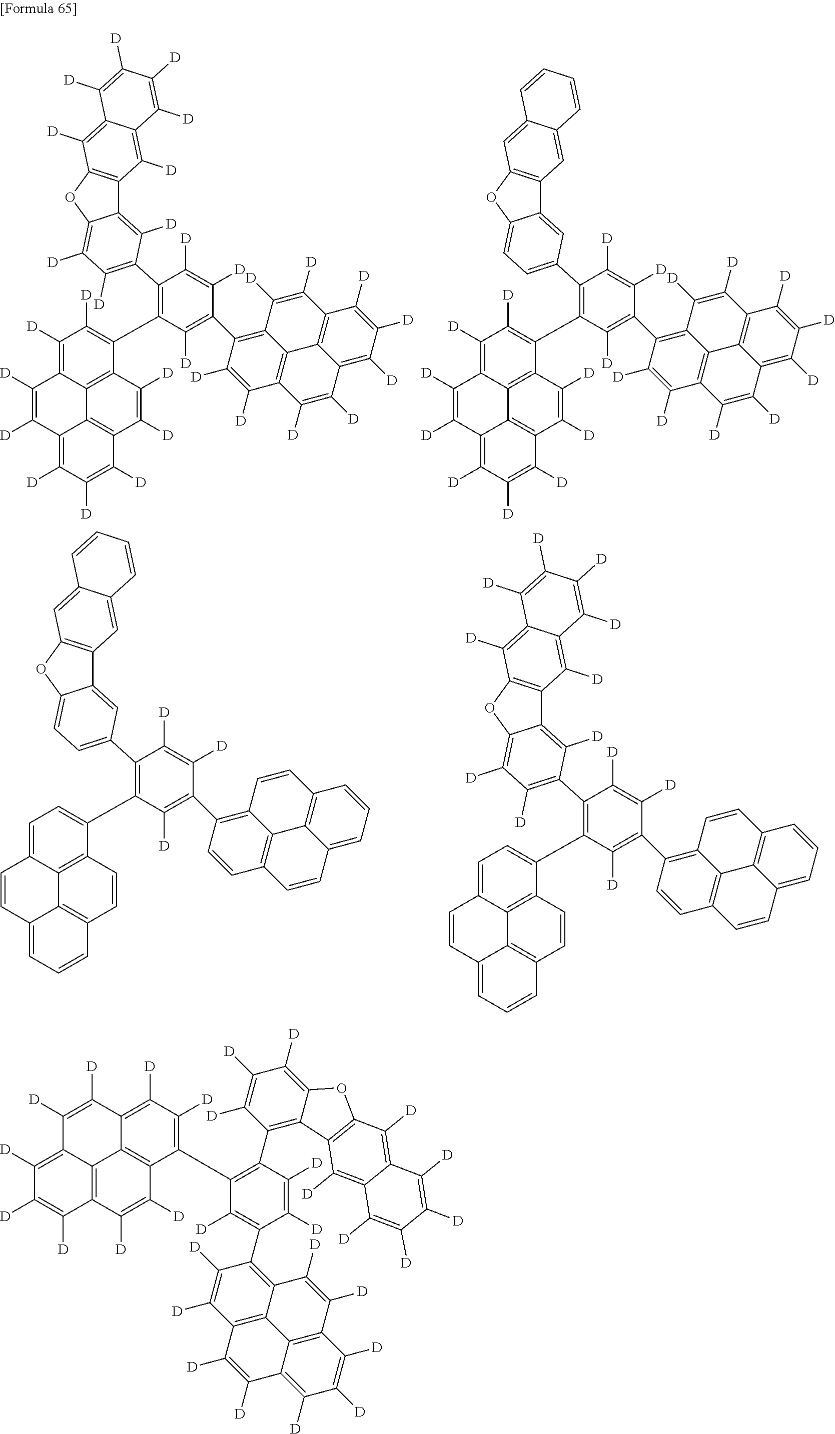

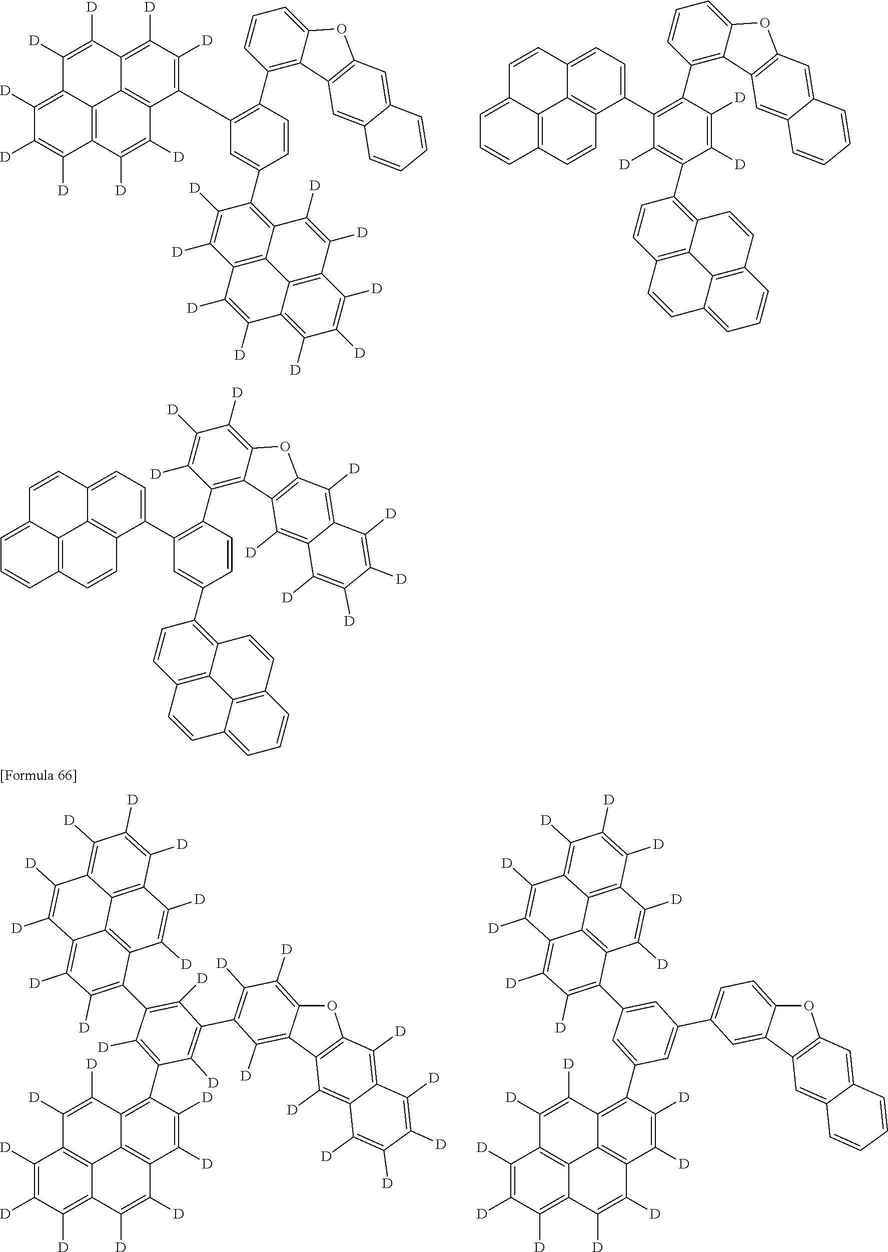

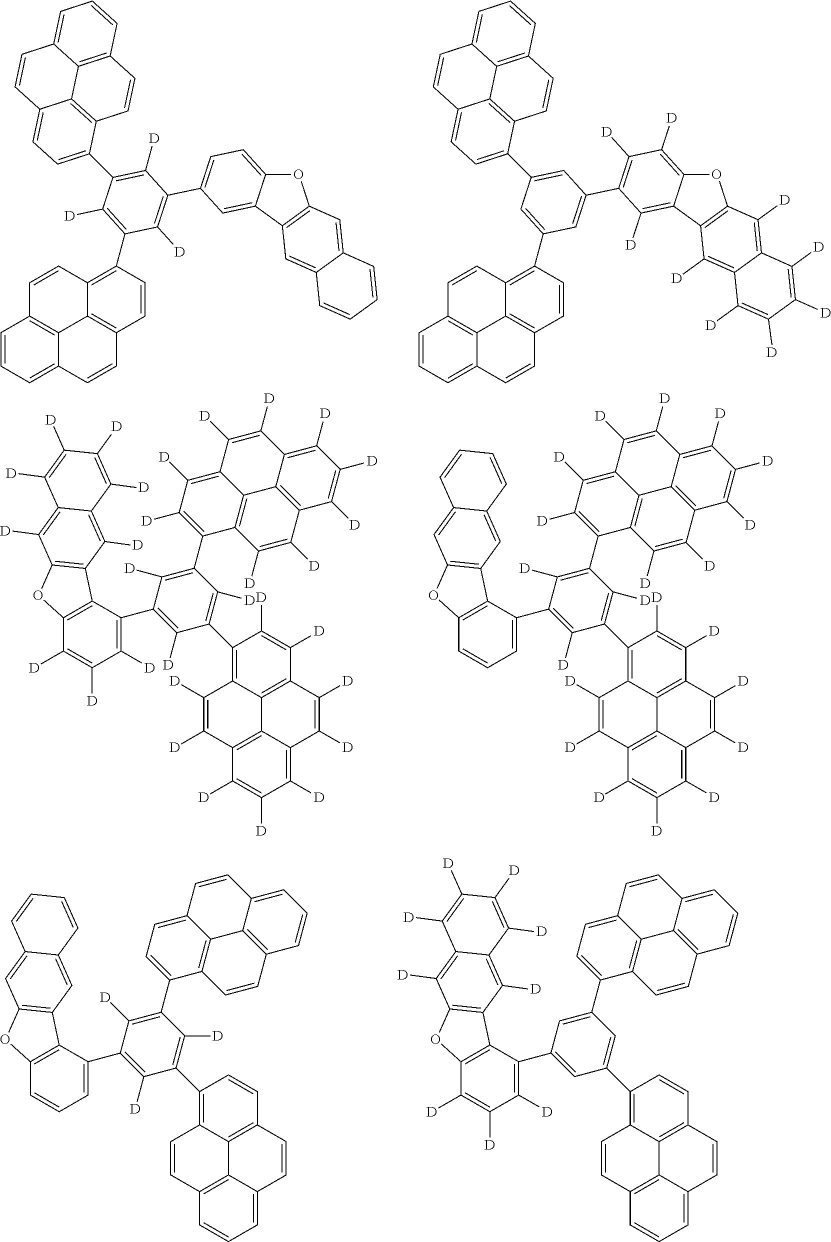

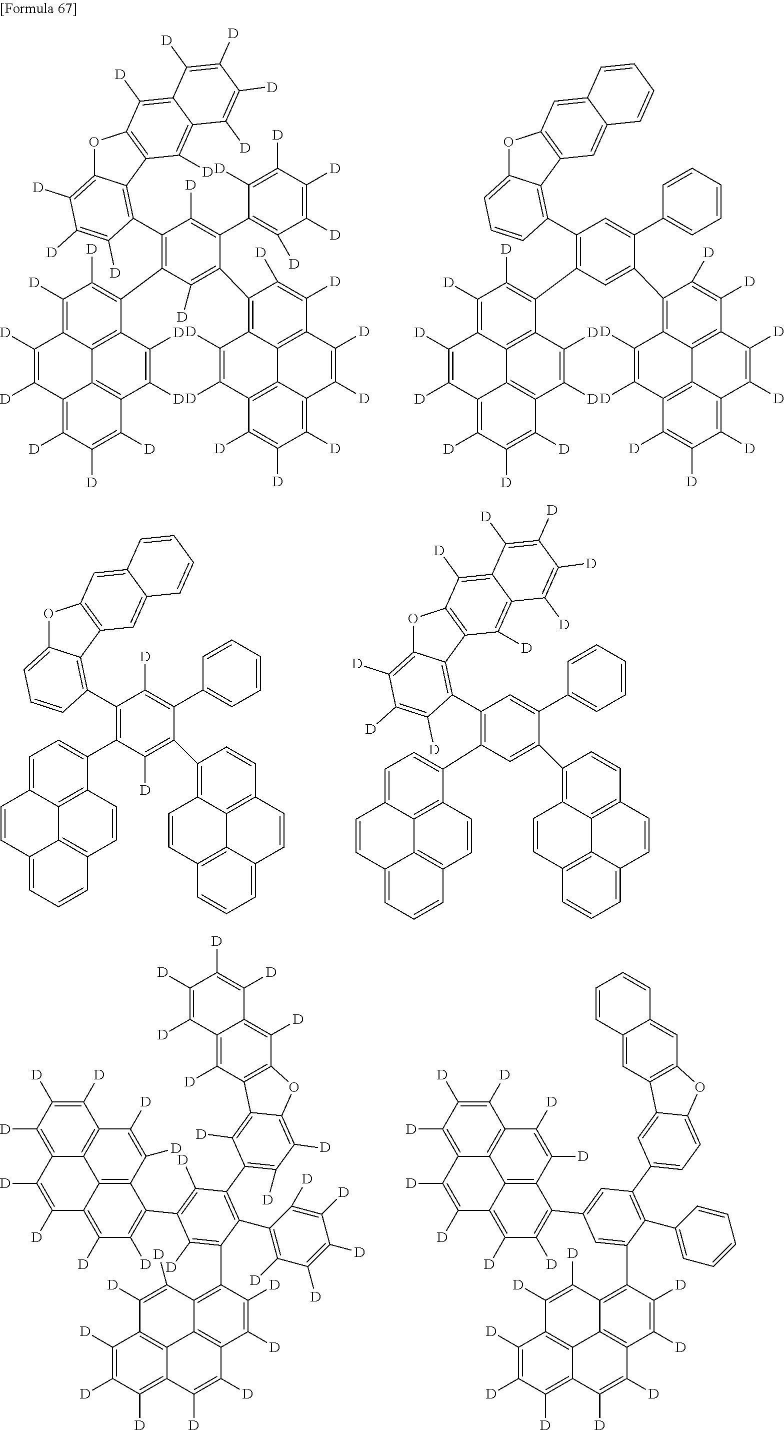

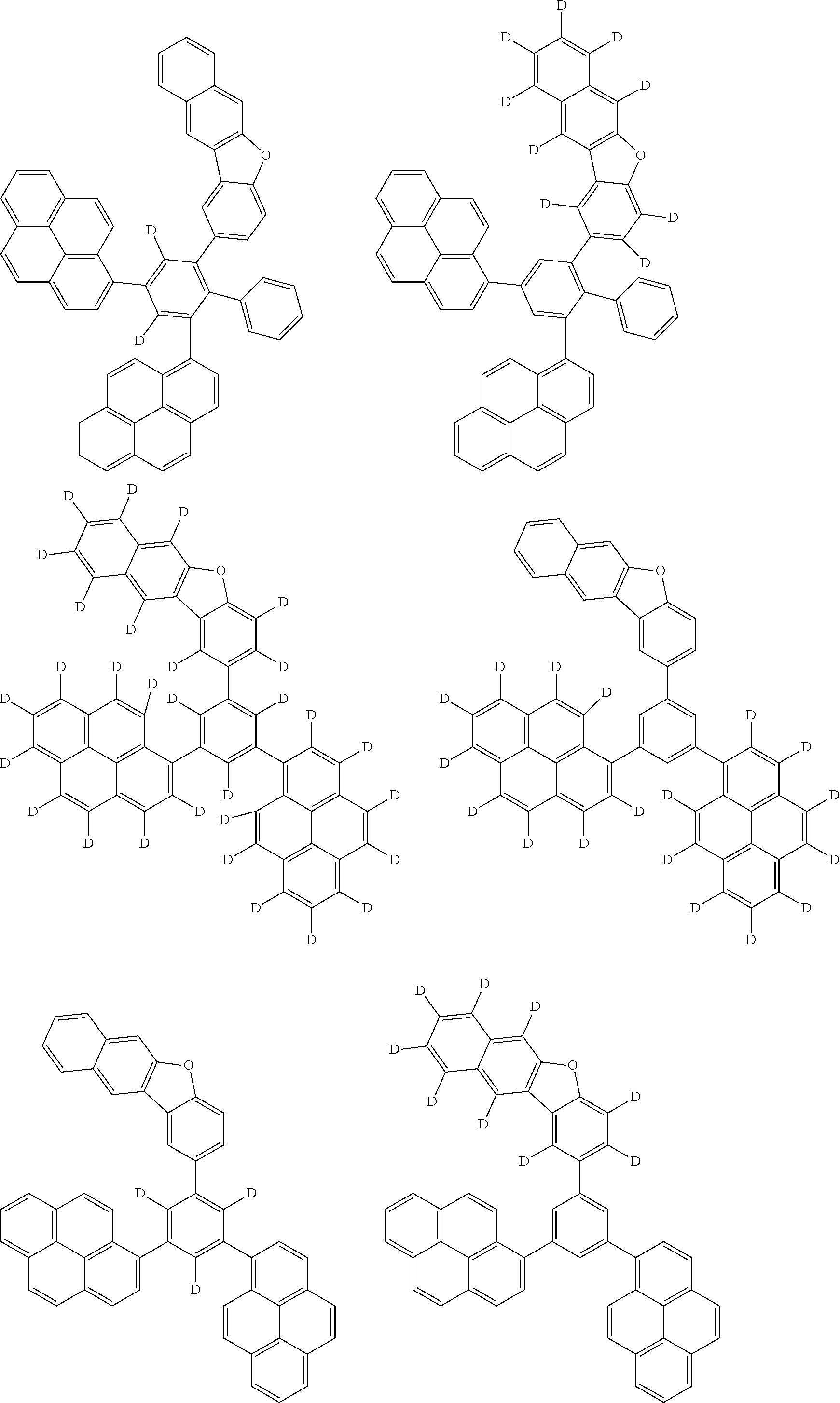

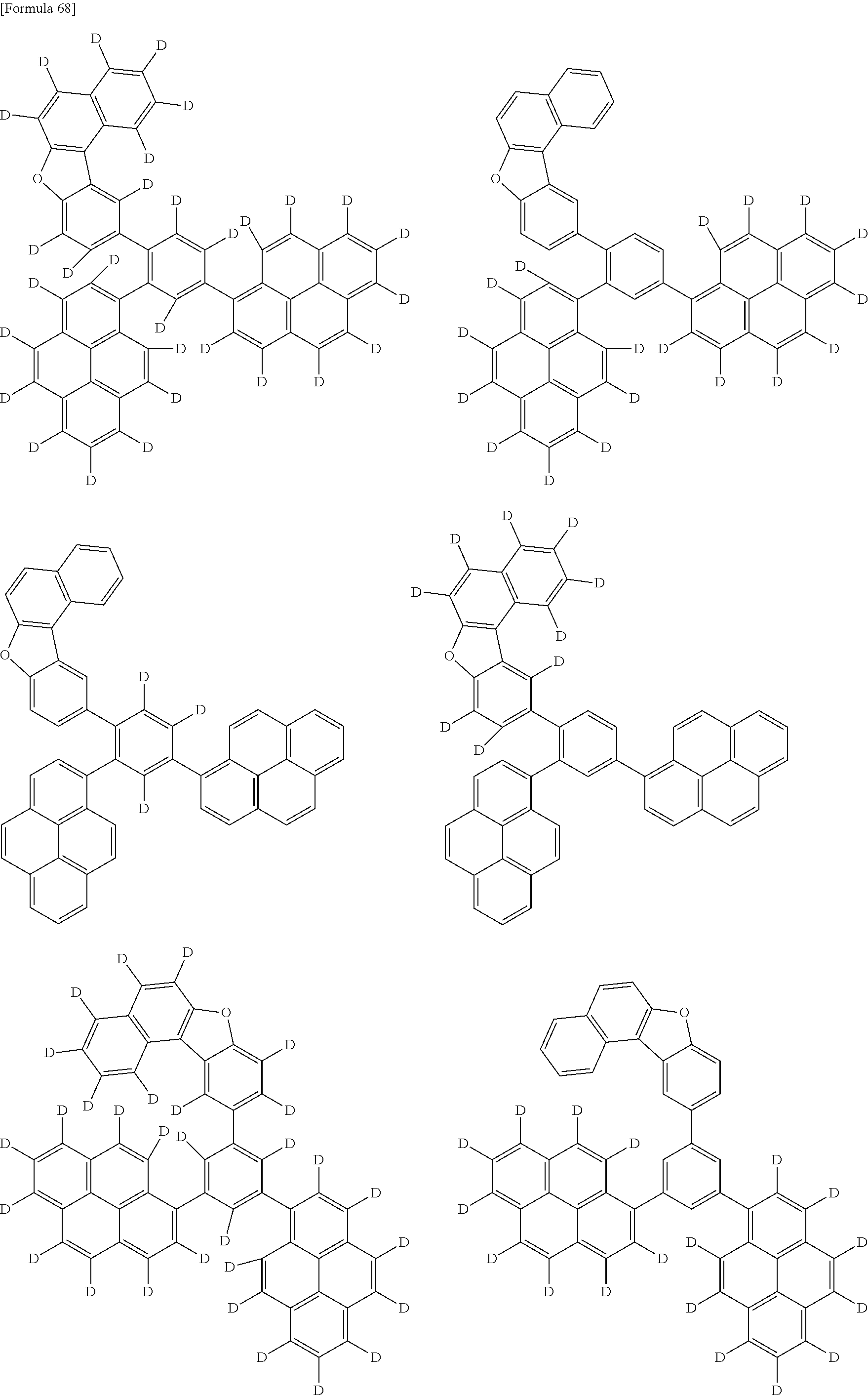

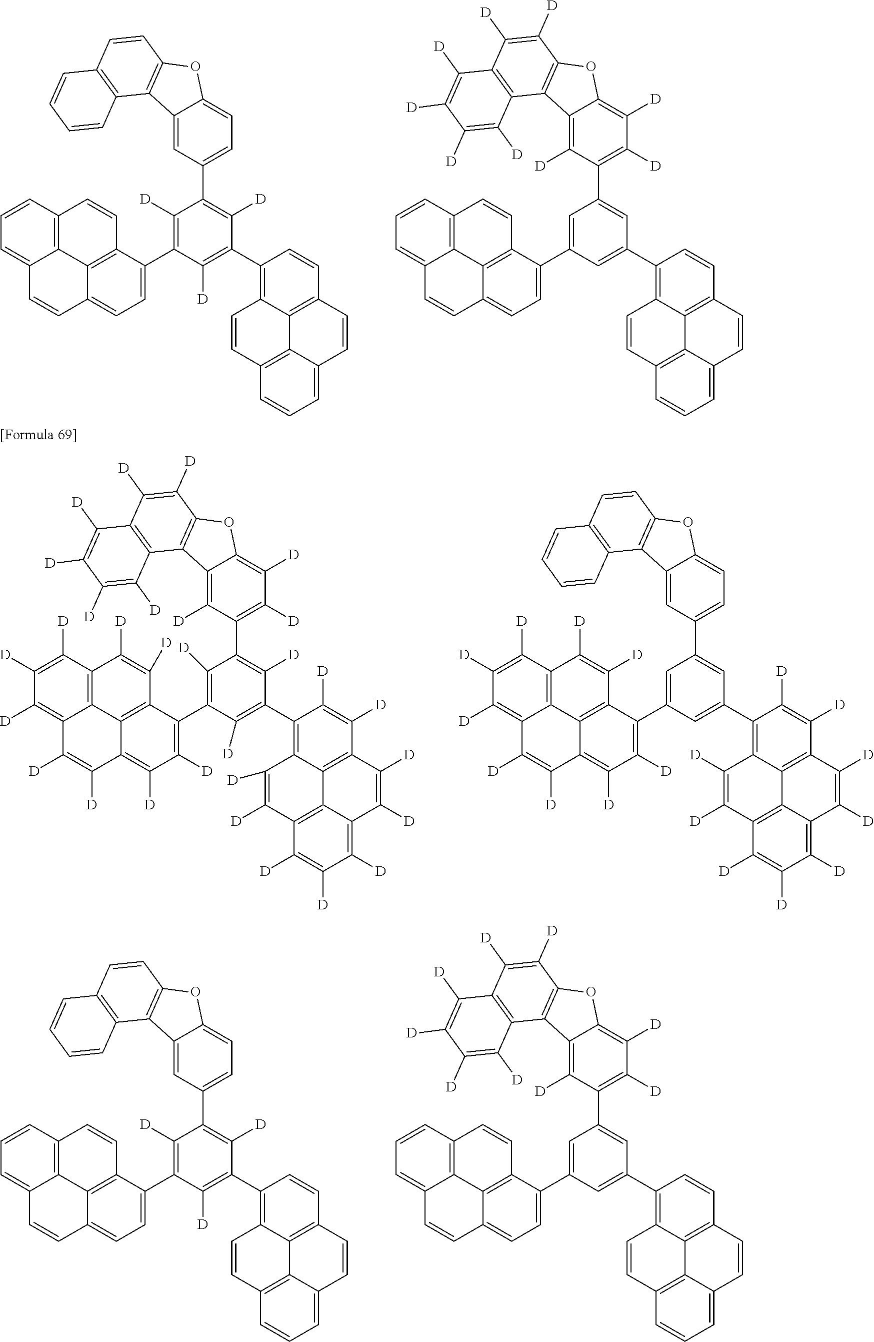

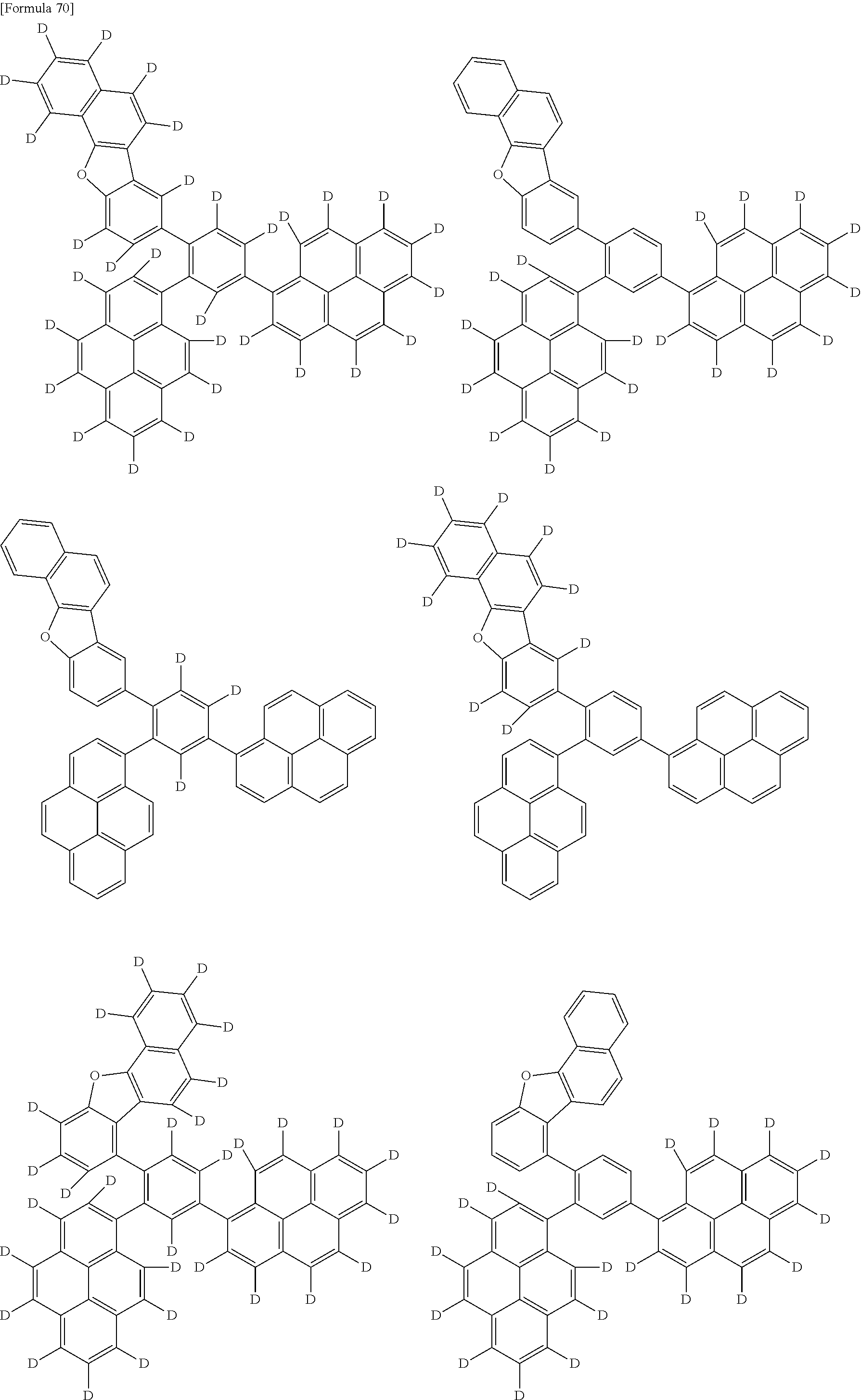

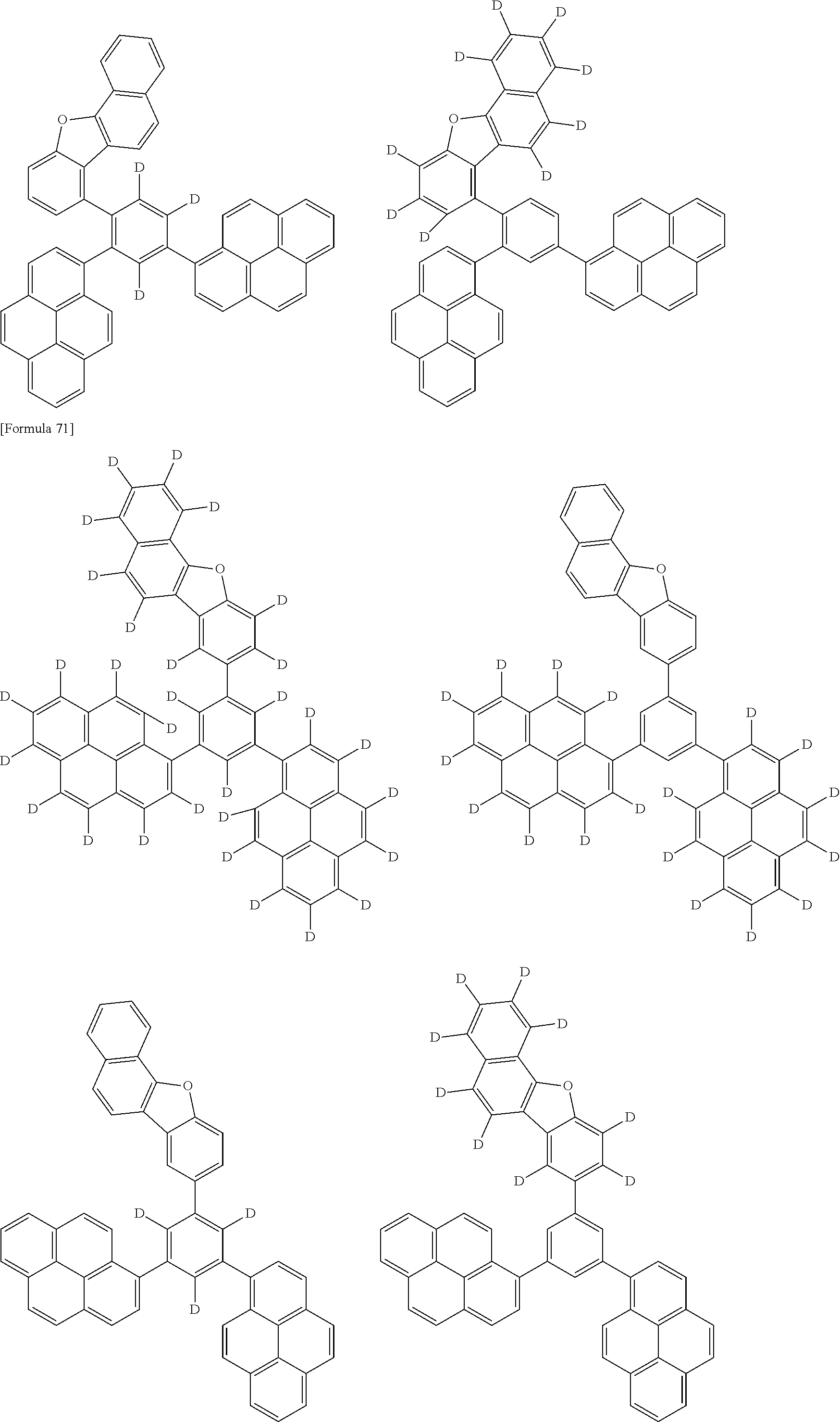

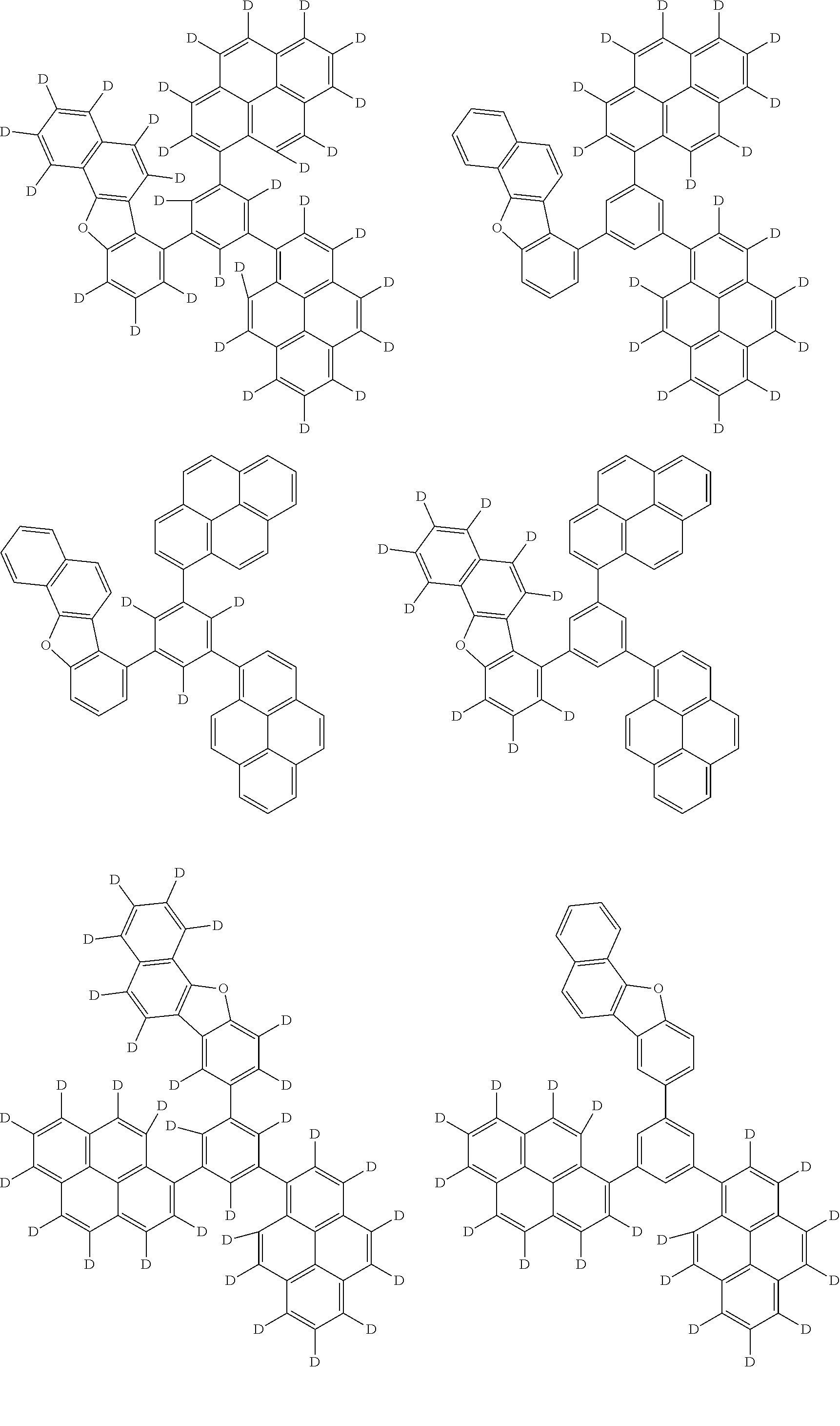

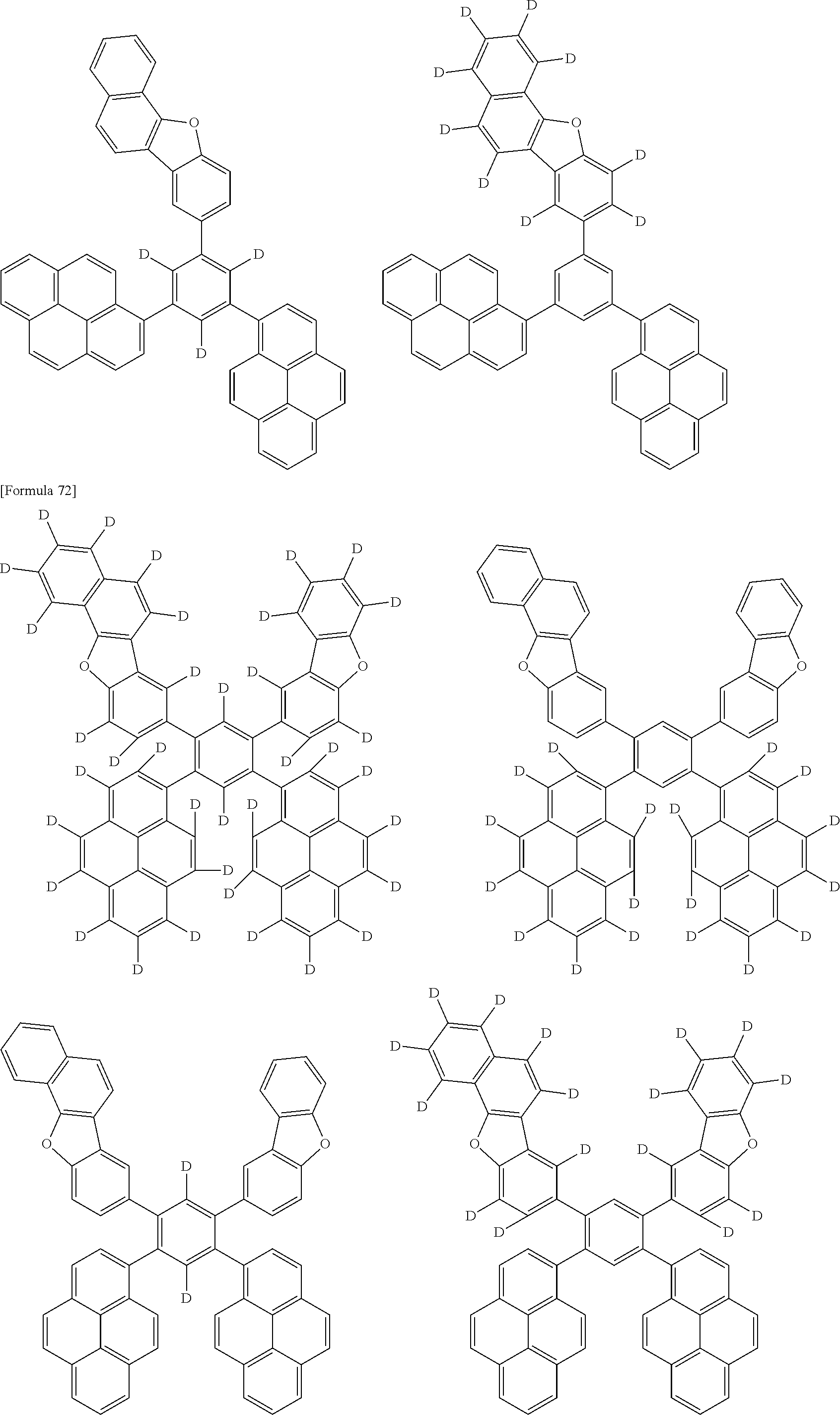

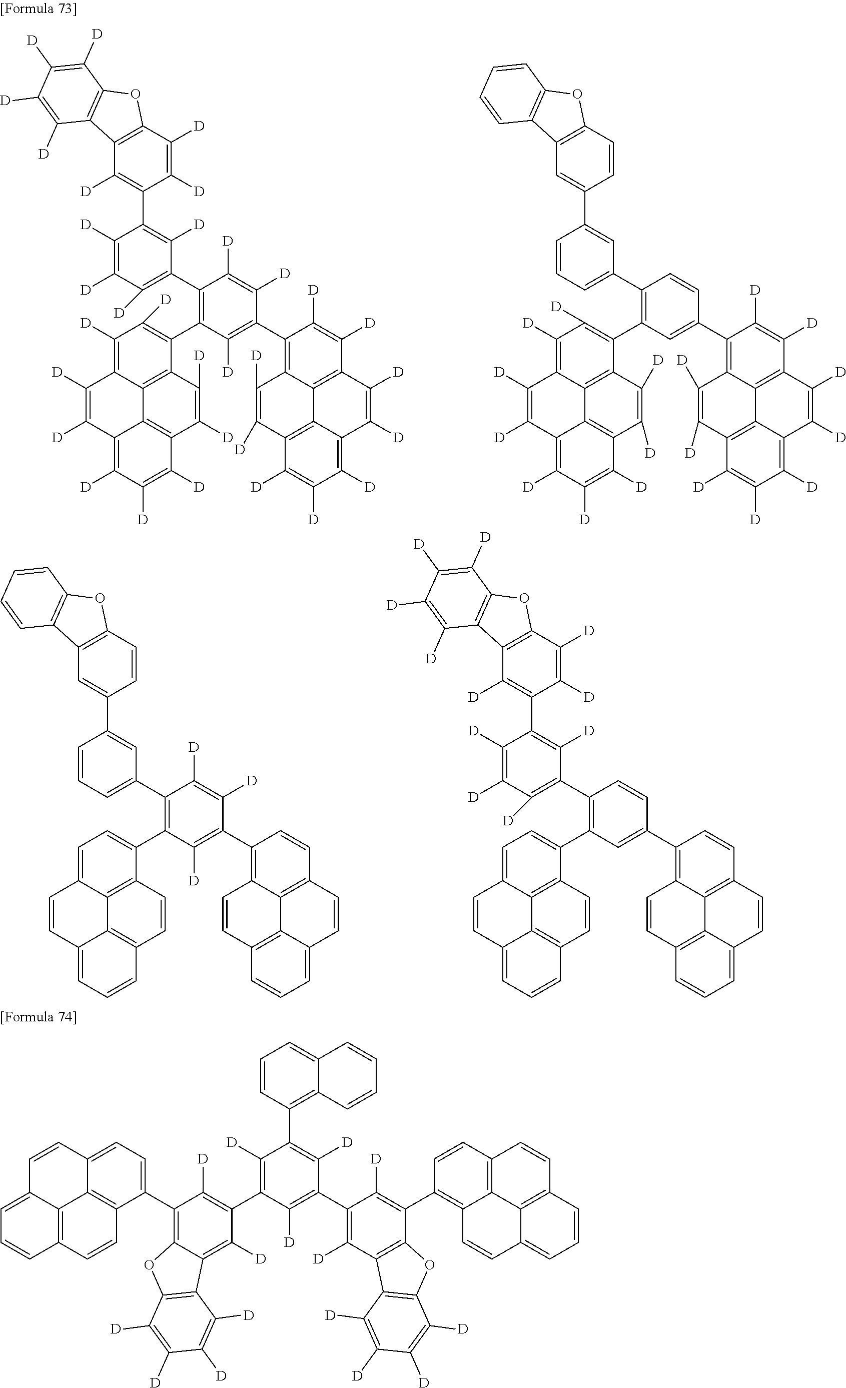

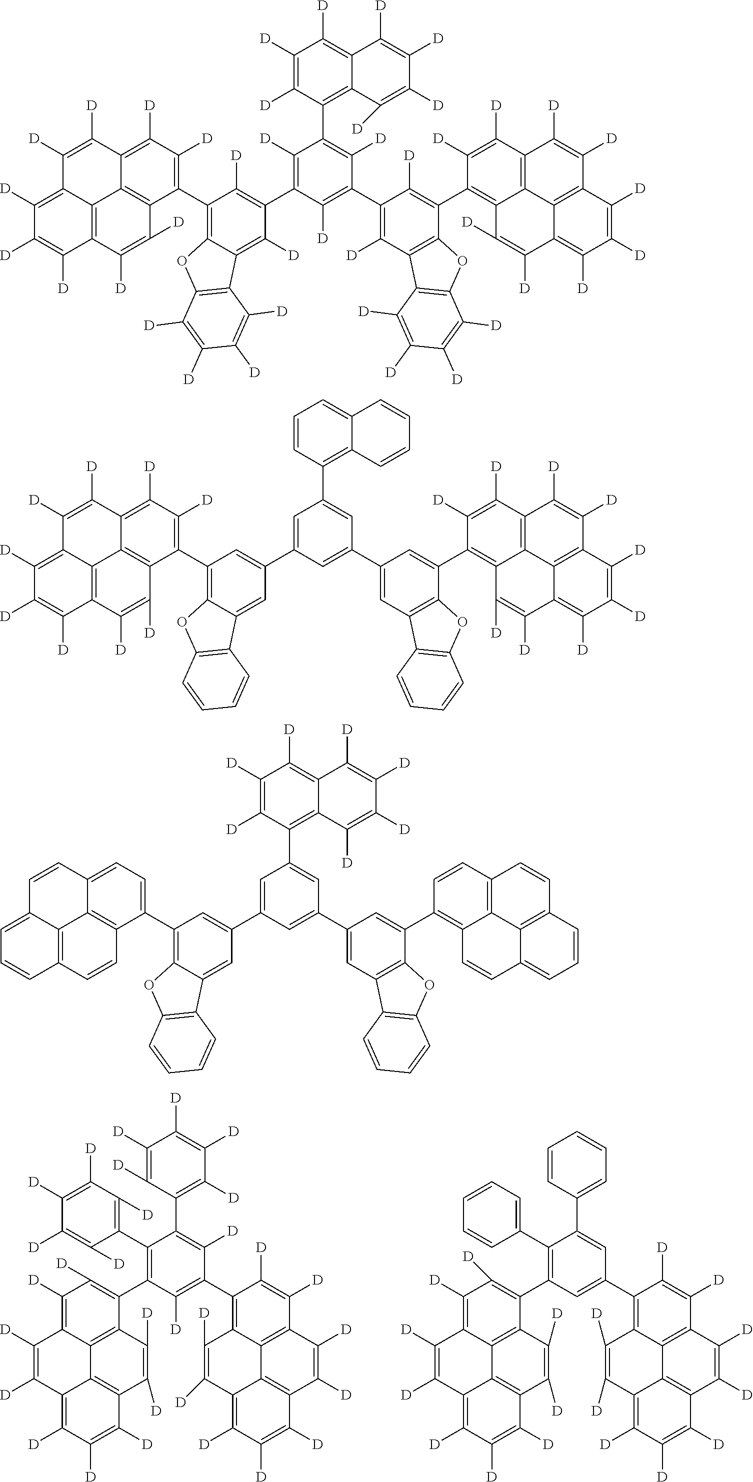

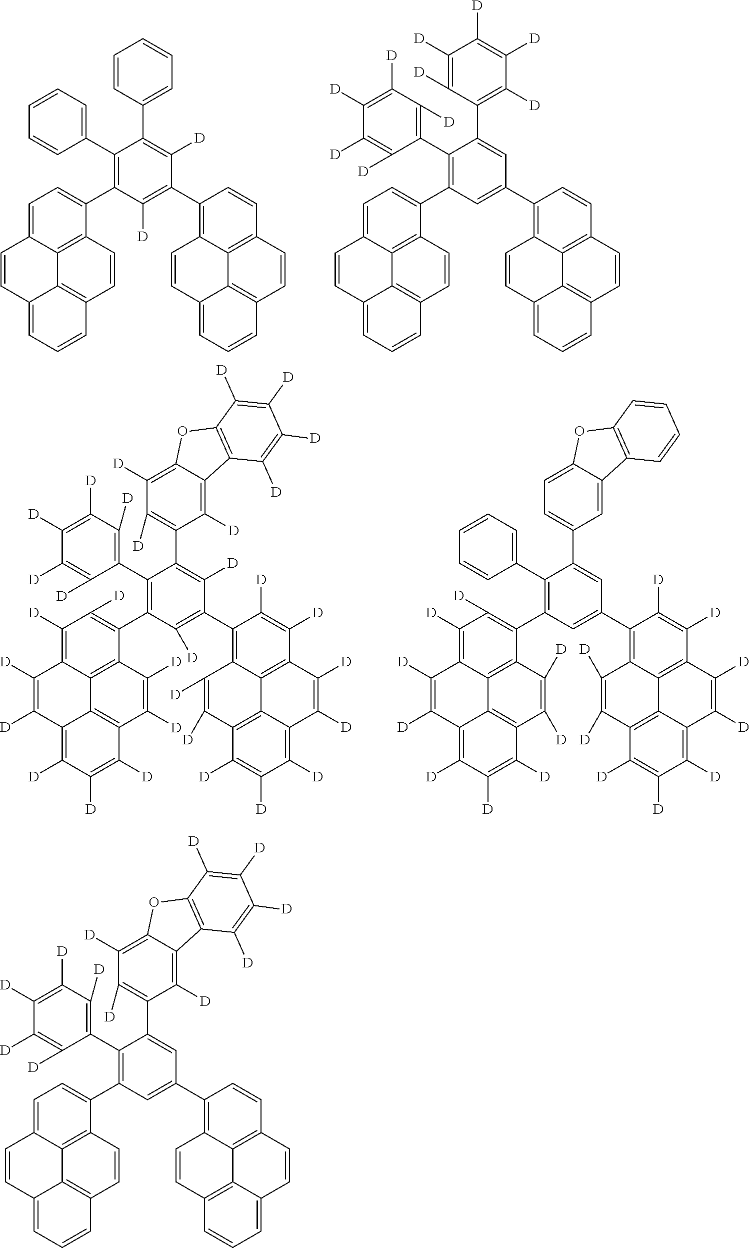

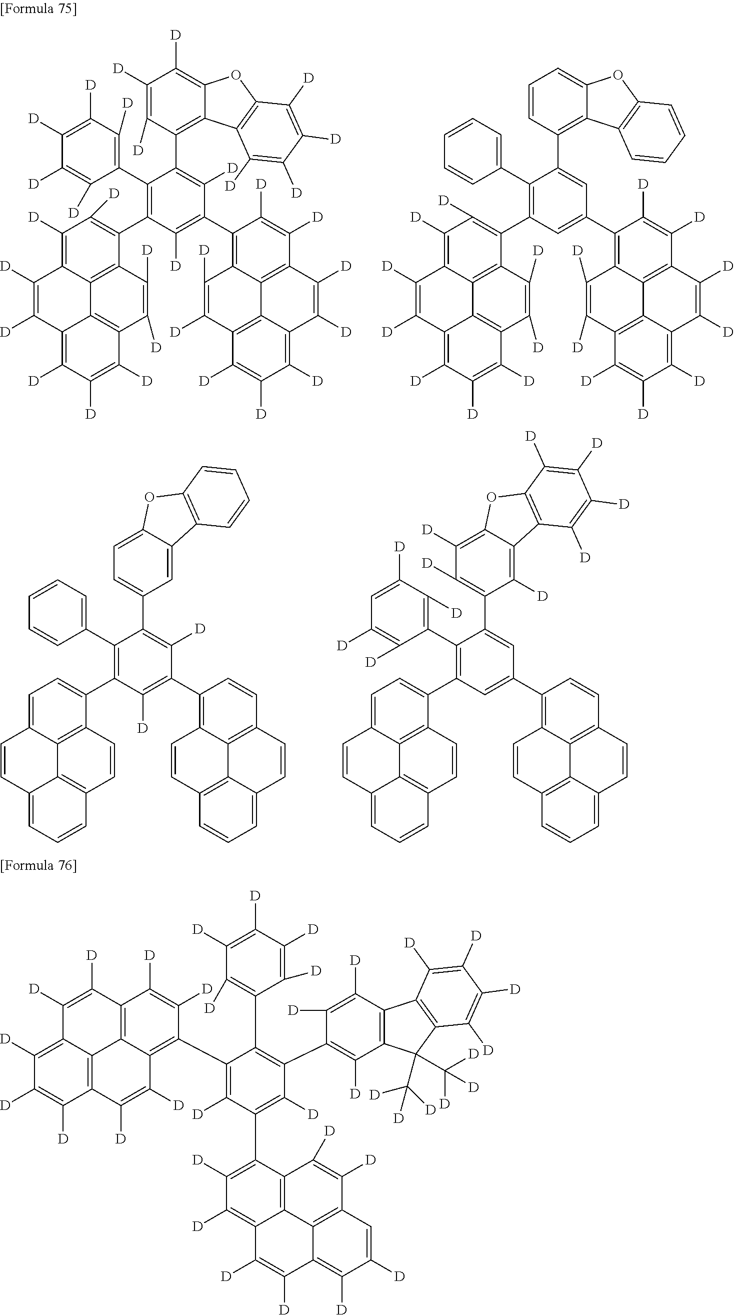

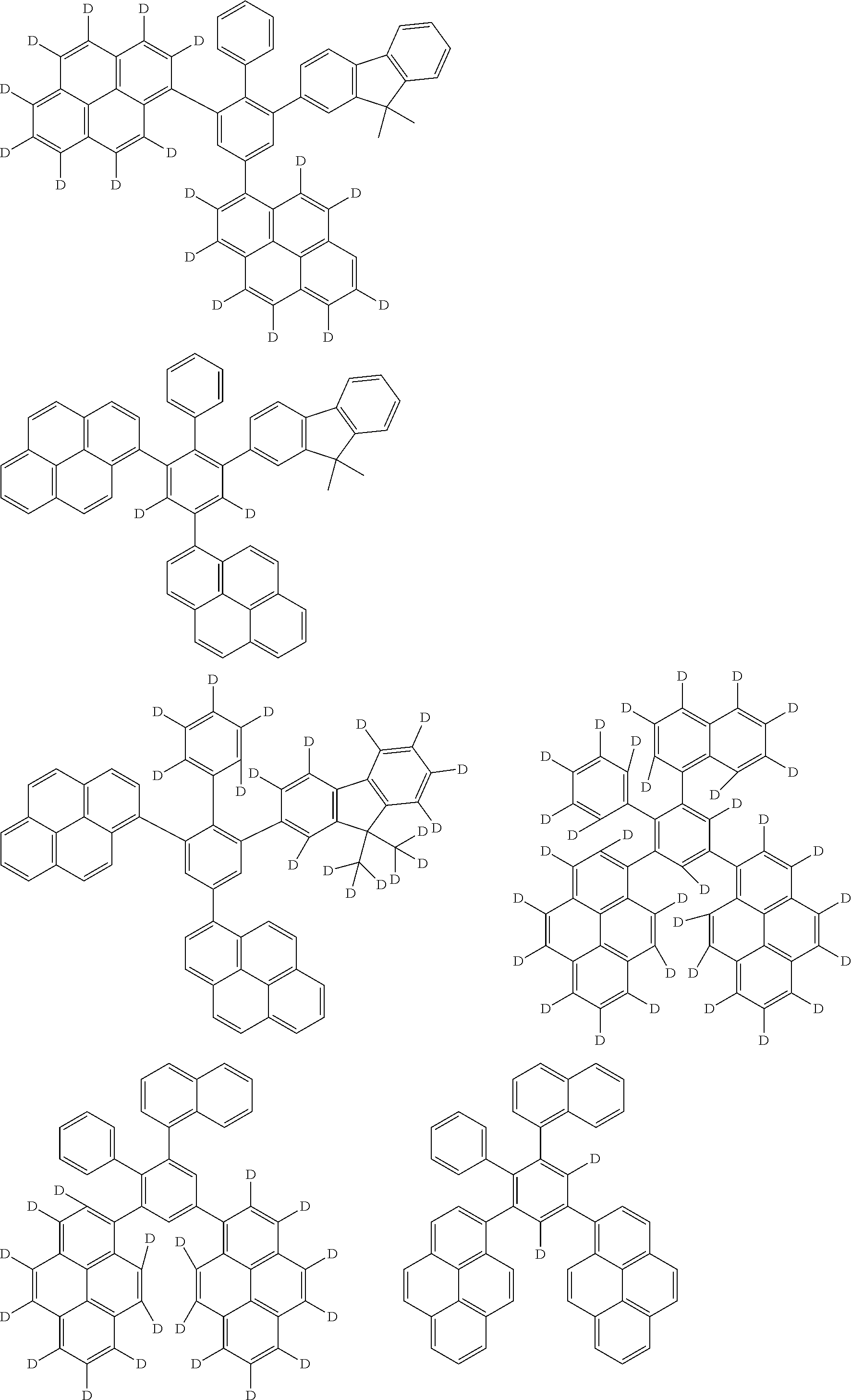

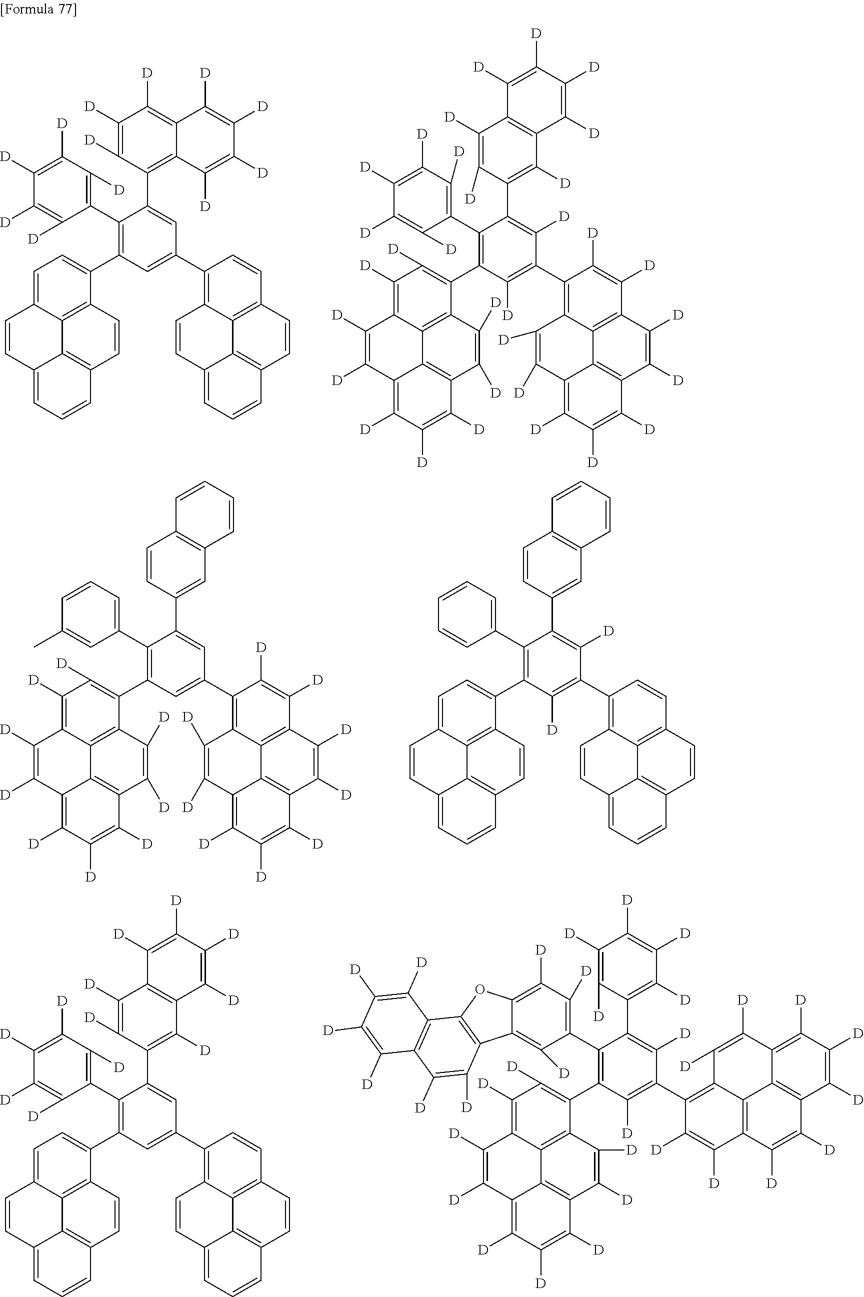

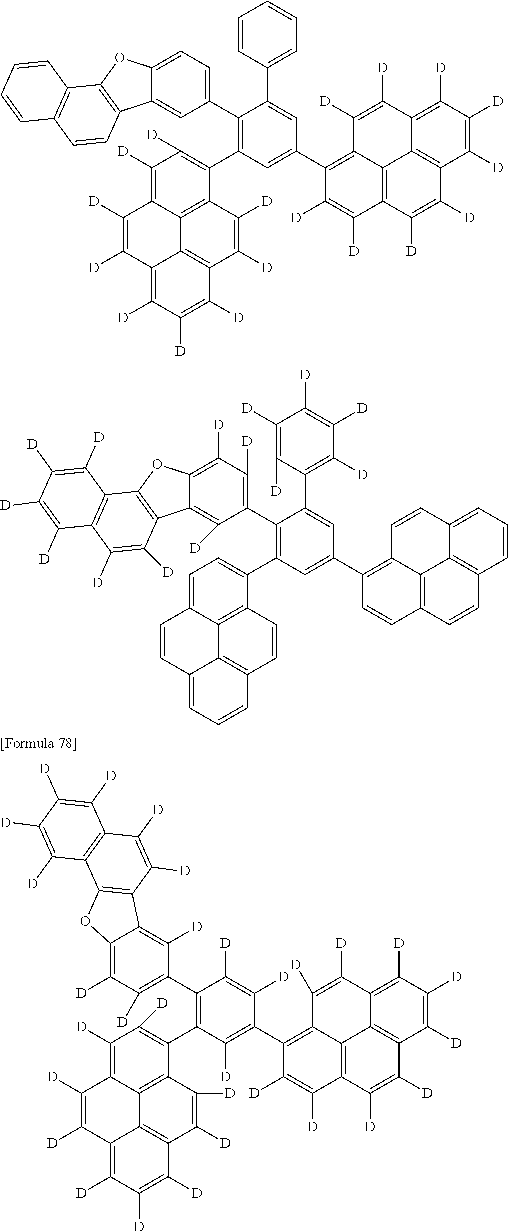

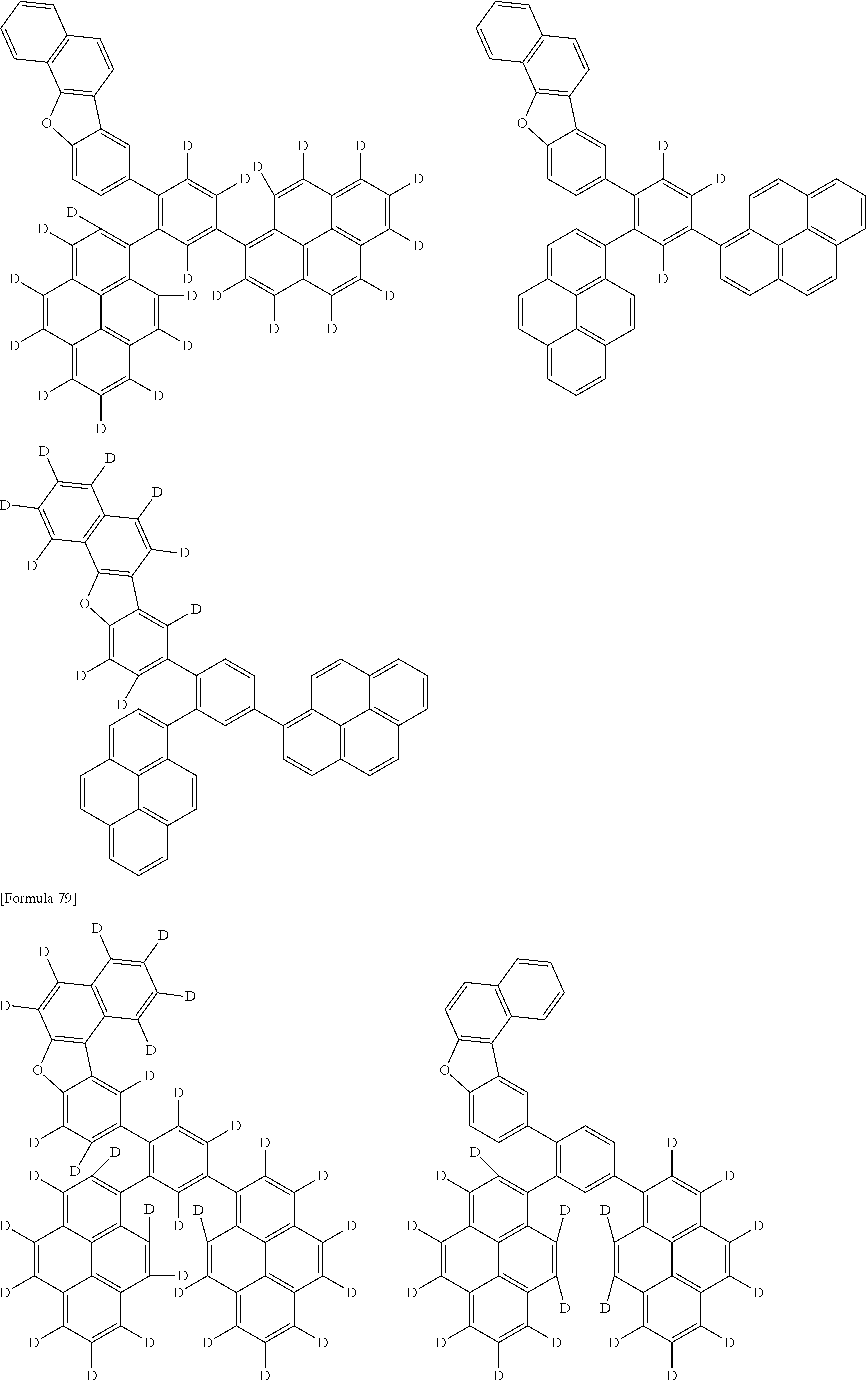

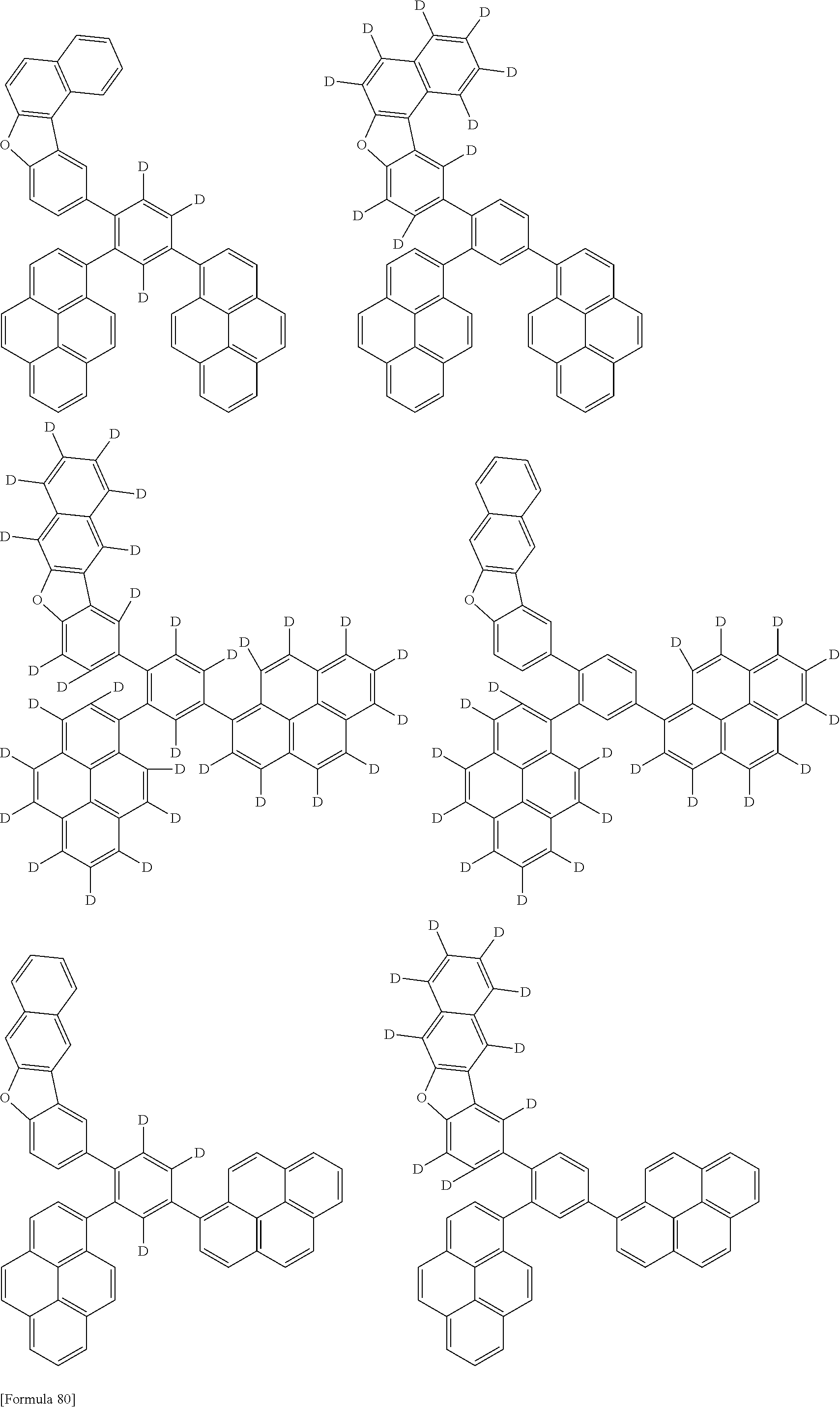

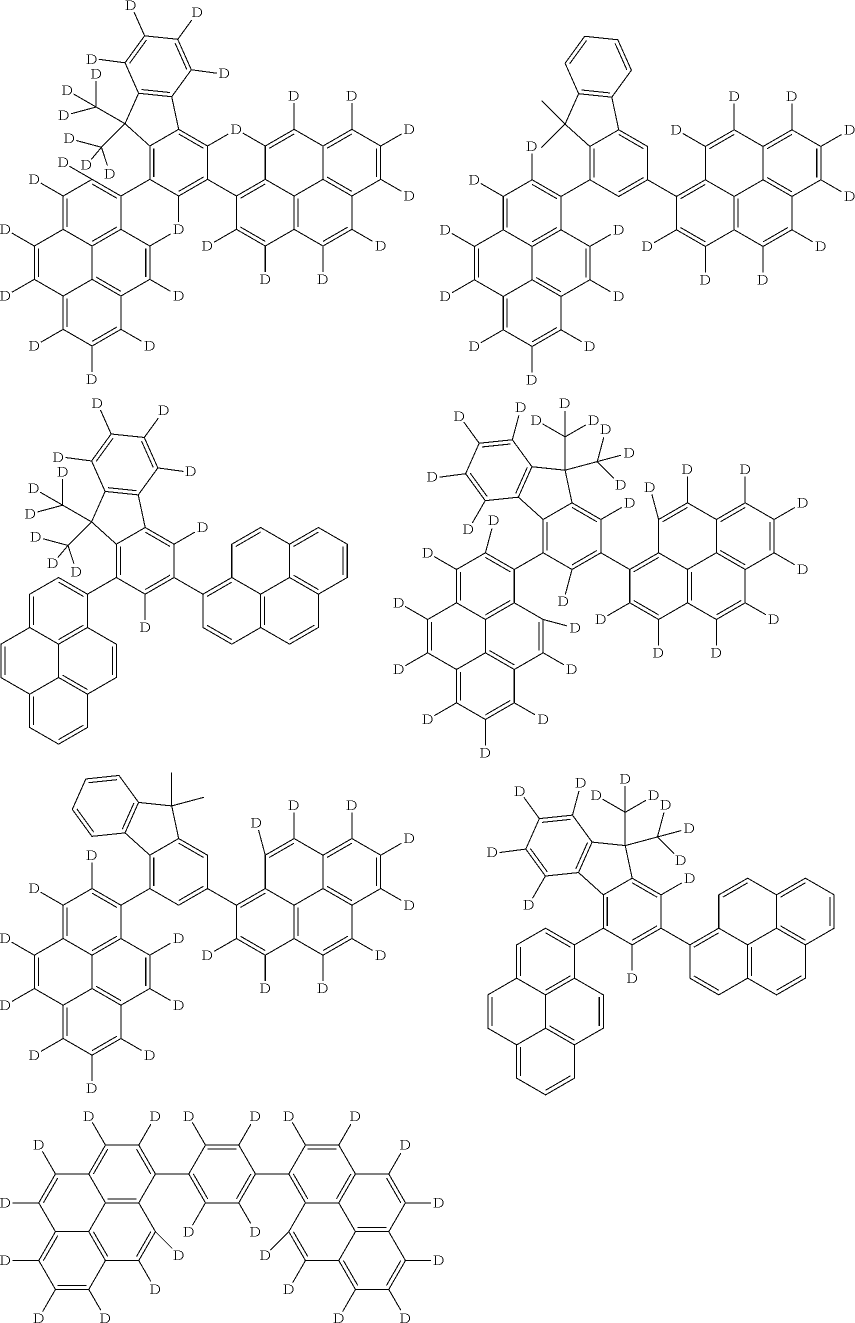

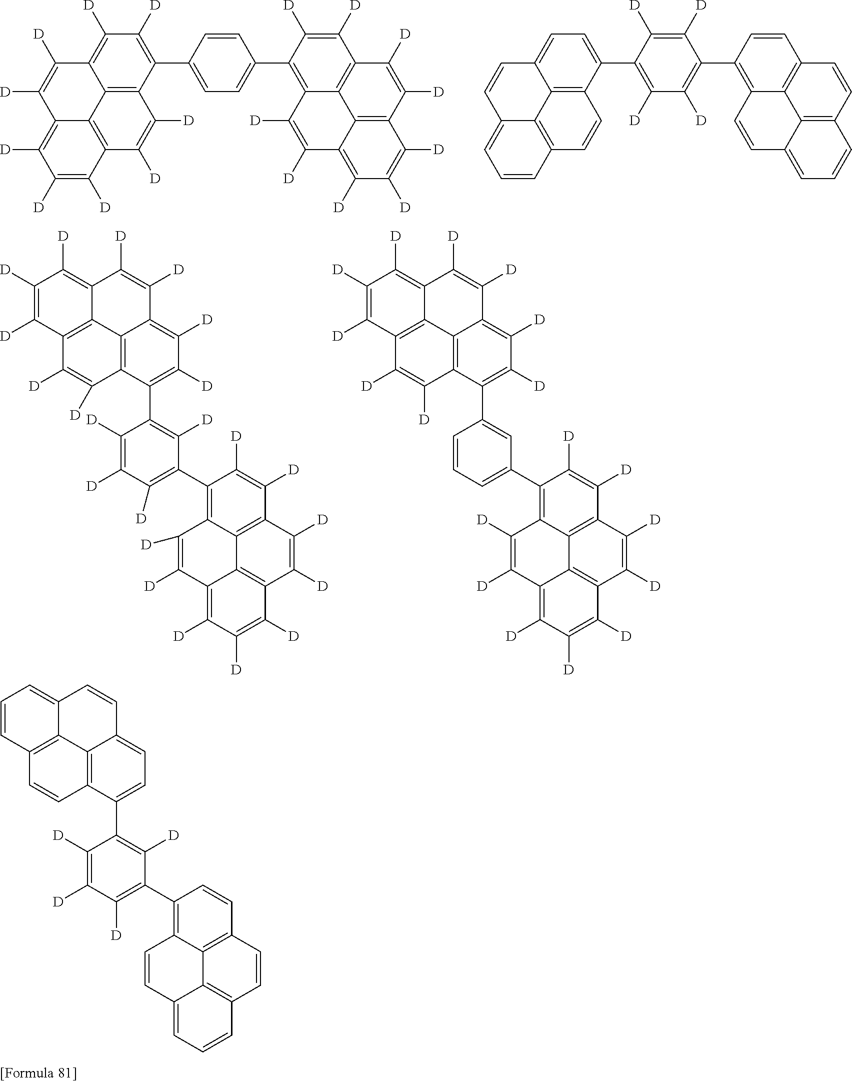

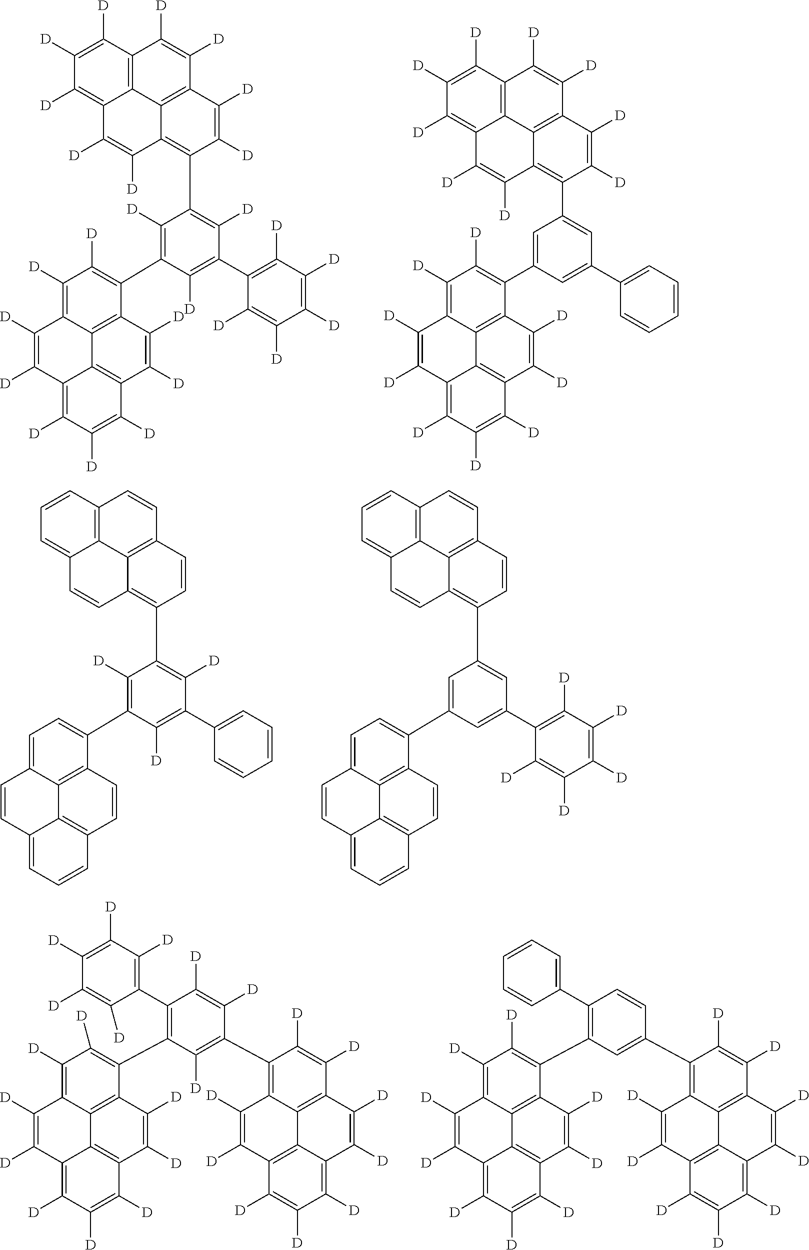

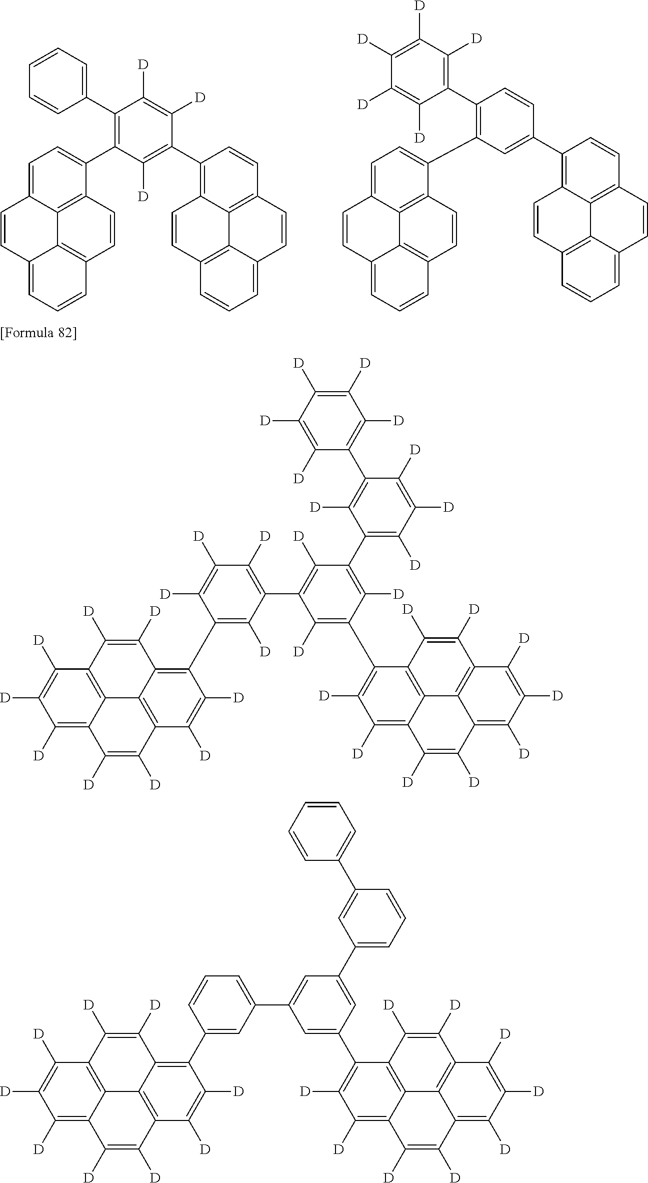

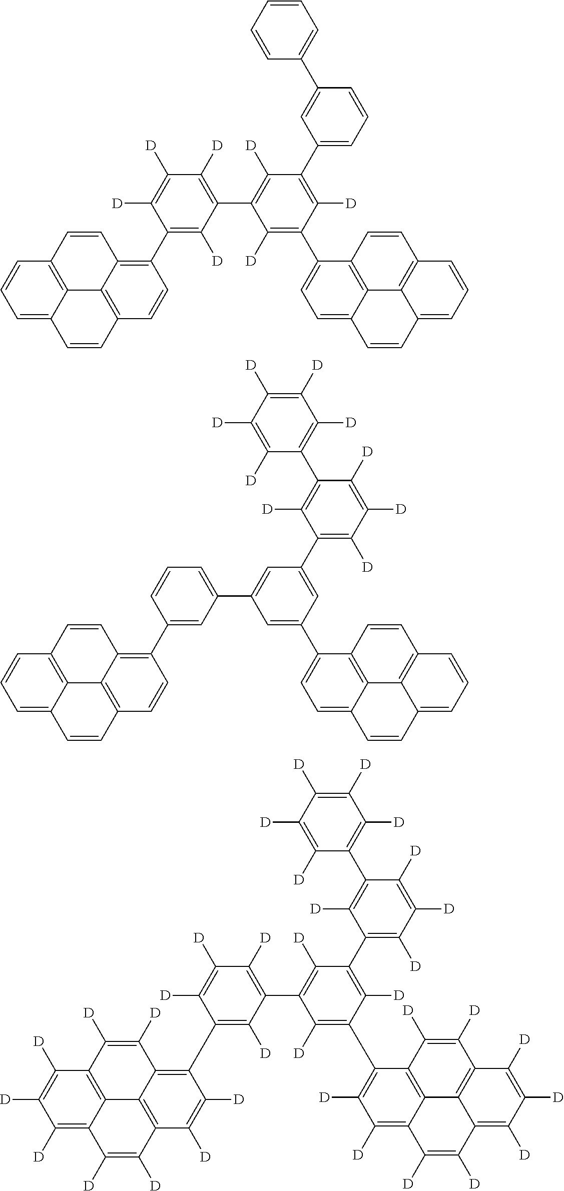

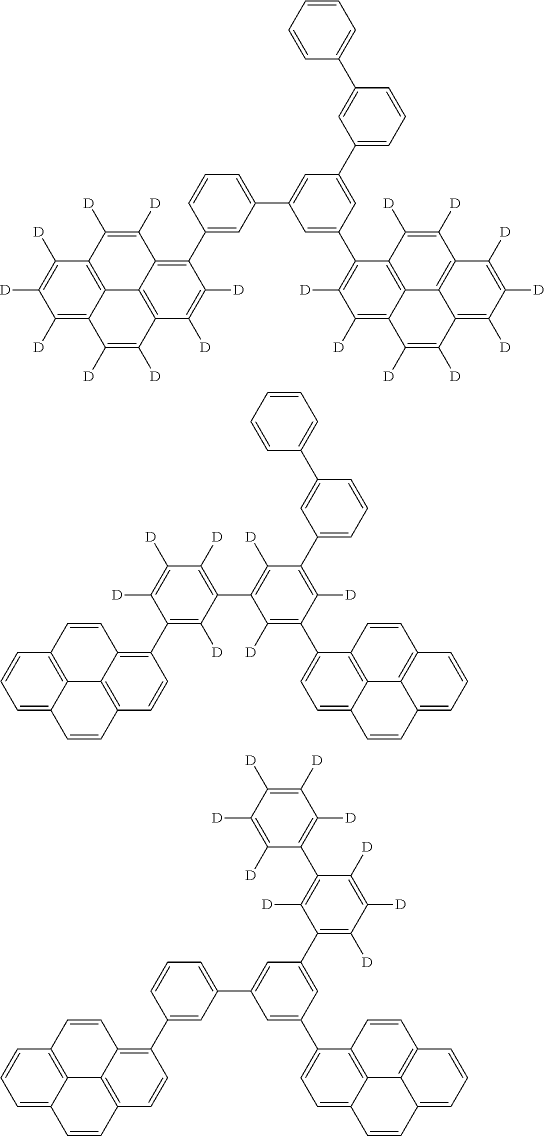 [SMILES: [2H]c1c([2H])c2c([2H])c([2H])c3c([2H])c([2H])c(-c4cccc(-c5cc(-c6cccc(-c7ccccc7)c6)cc(-c6c([2H])c([2H])c7c([2H])c([2H])c8c([2H])c([2H])c([2H])c9c([2H])c([2H])c6c7c89)c5)c4)c4c([2H])c([2H])c(c1[2H])c2c34 ; [2H]c1c([2H])c(-c2c([2H])c(-c3cccc(-c4ccccc4)c3)c([2H])c(-c3ccc4ccc5cccc6ccc3c4c56)c2[2H])c([2H])c(-c2ccc3ccc4cccc5ccc2c3c45)c1[2H] ; [2H]c1c([2H])c([2H])c(-c2c([2H])c([2H])c([2H])c(-c3cc(-c4cccc(-c5ccc6ccc7cccc8ccc5c6c78)c4)cc(-c4ccc5ccc6cccc7ccc4c5c67)c3)c2[2H])c([2H])c1[2H]]

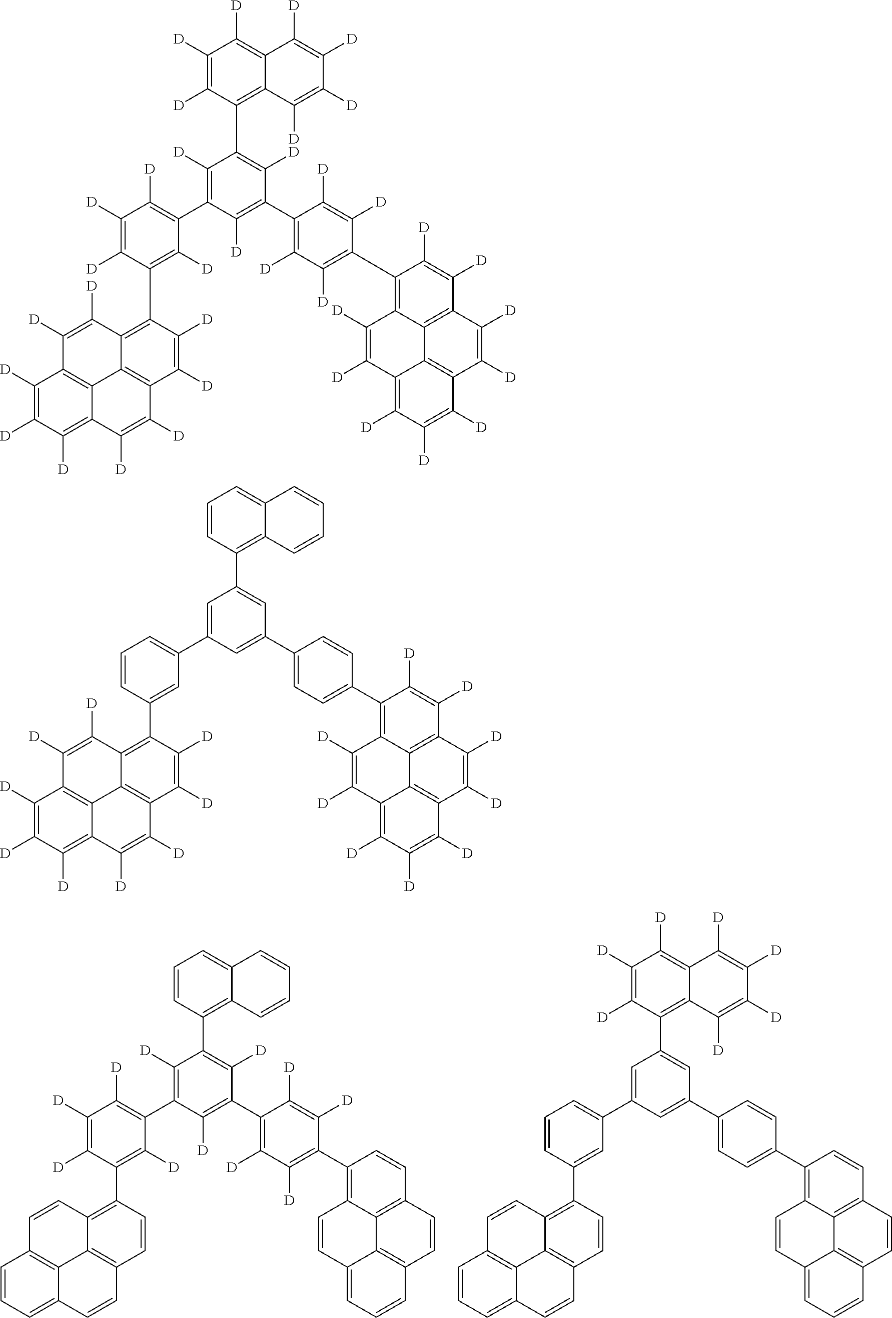

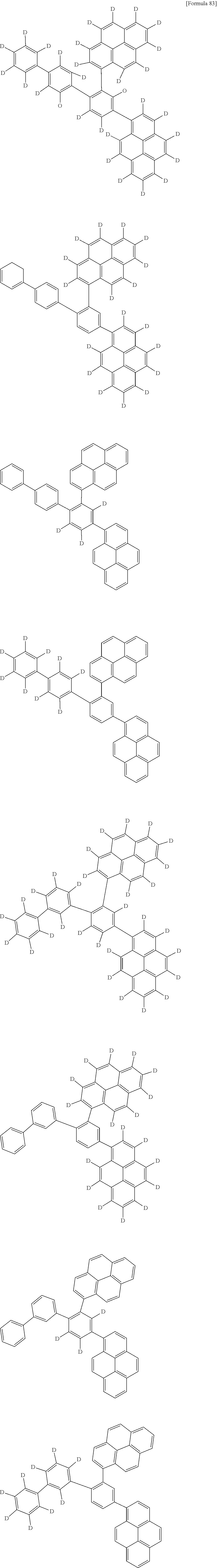

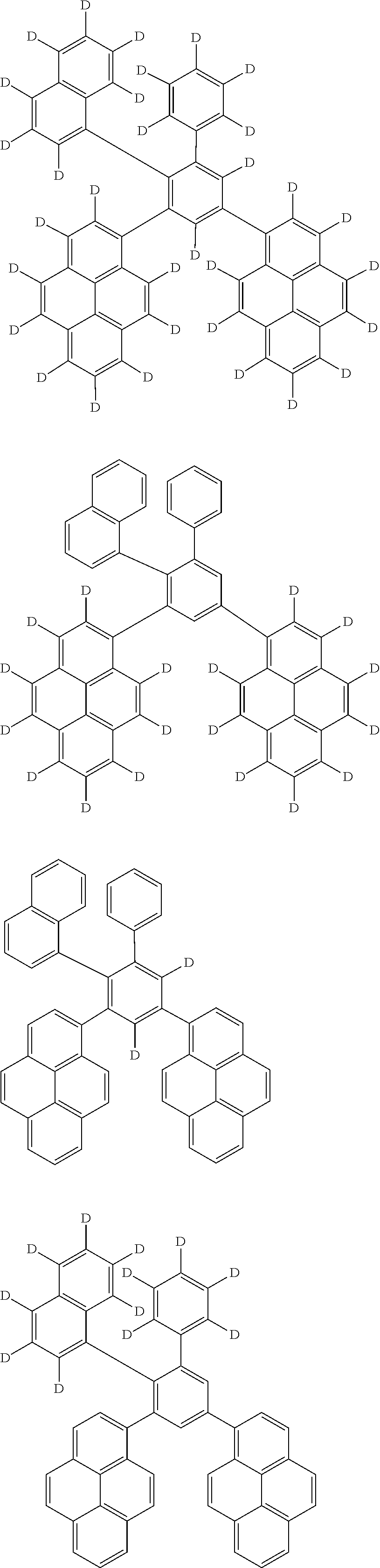

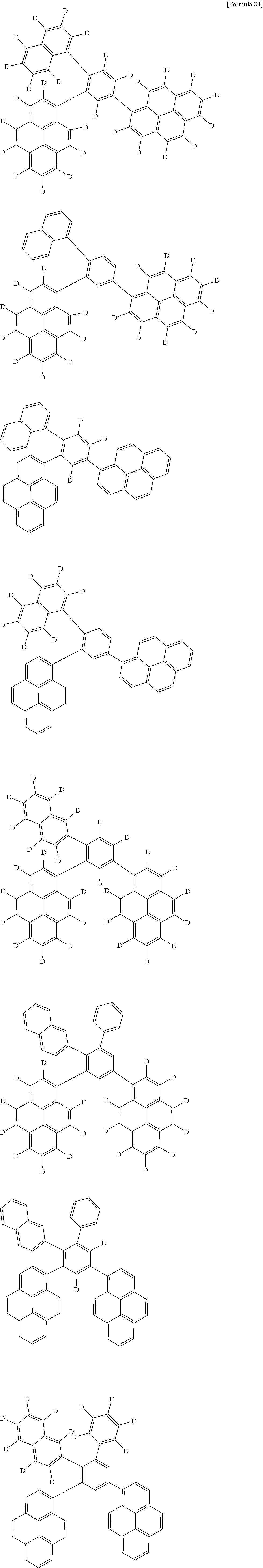

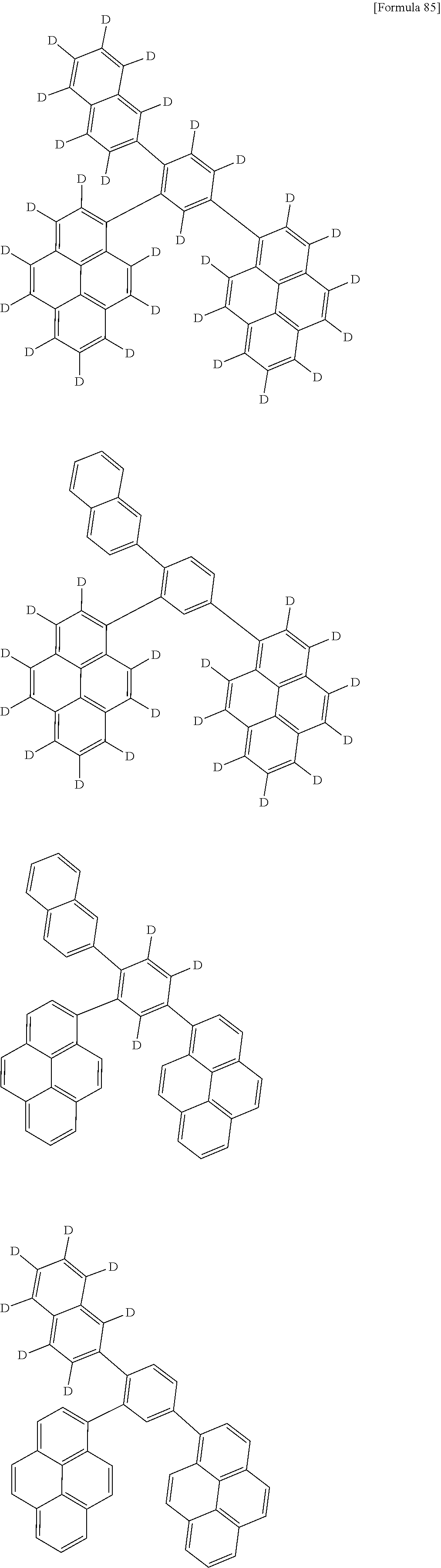

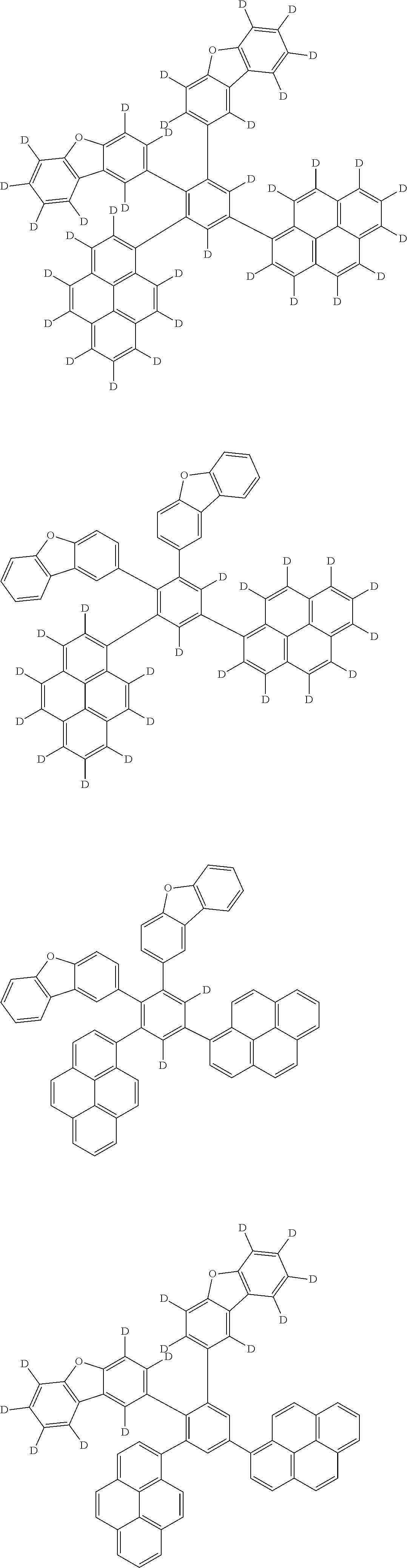

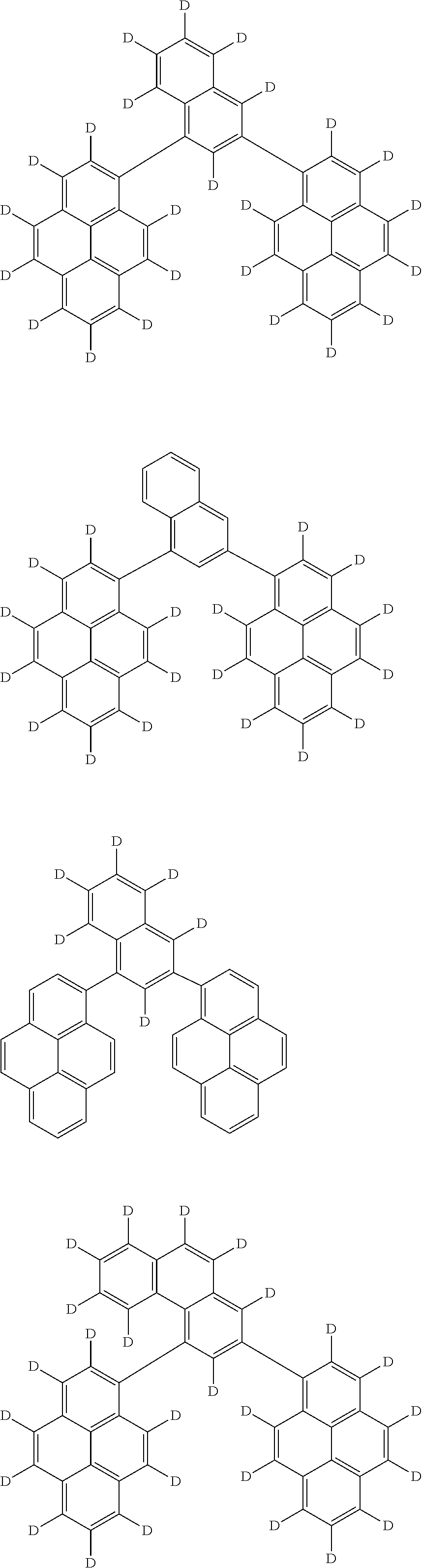

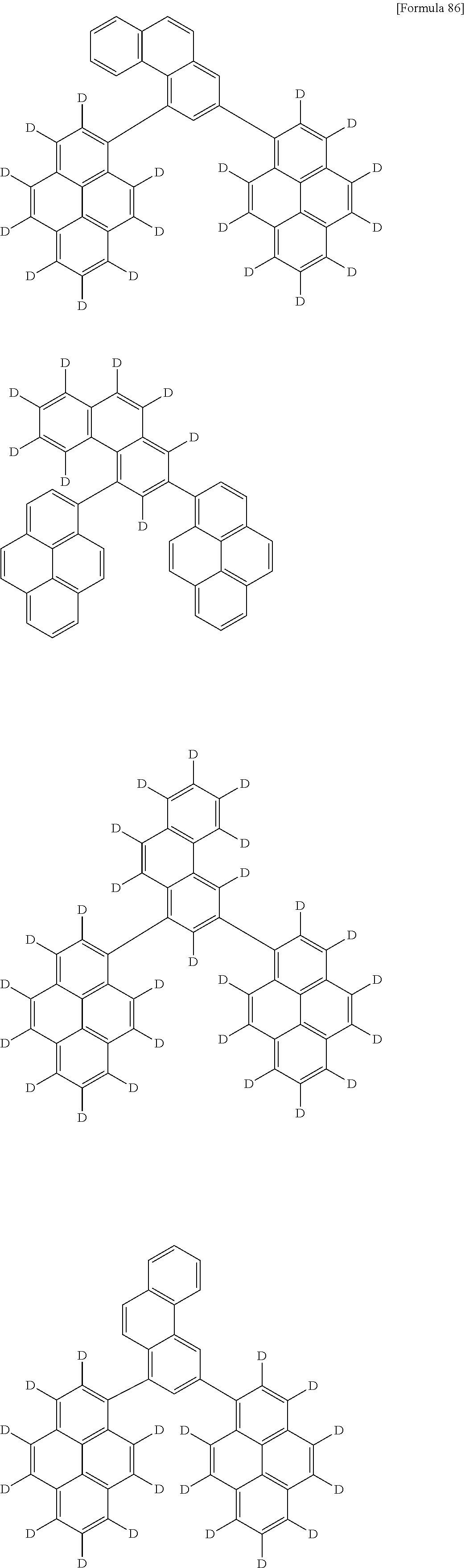

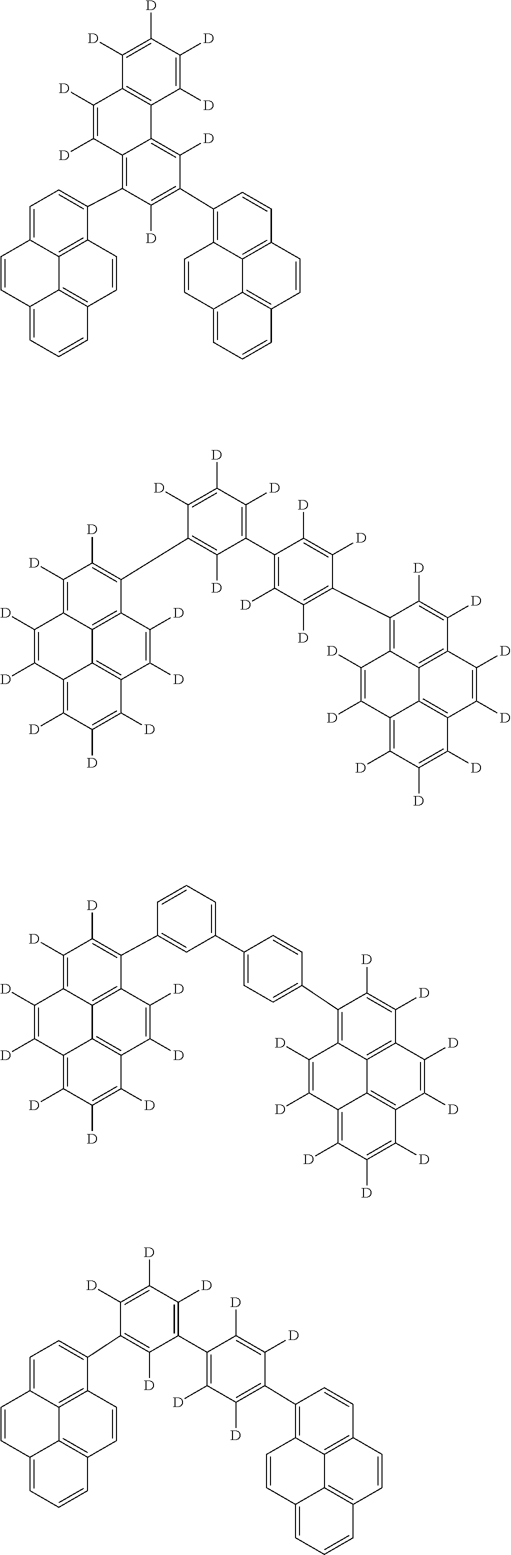

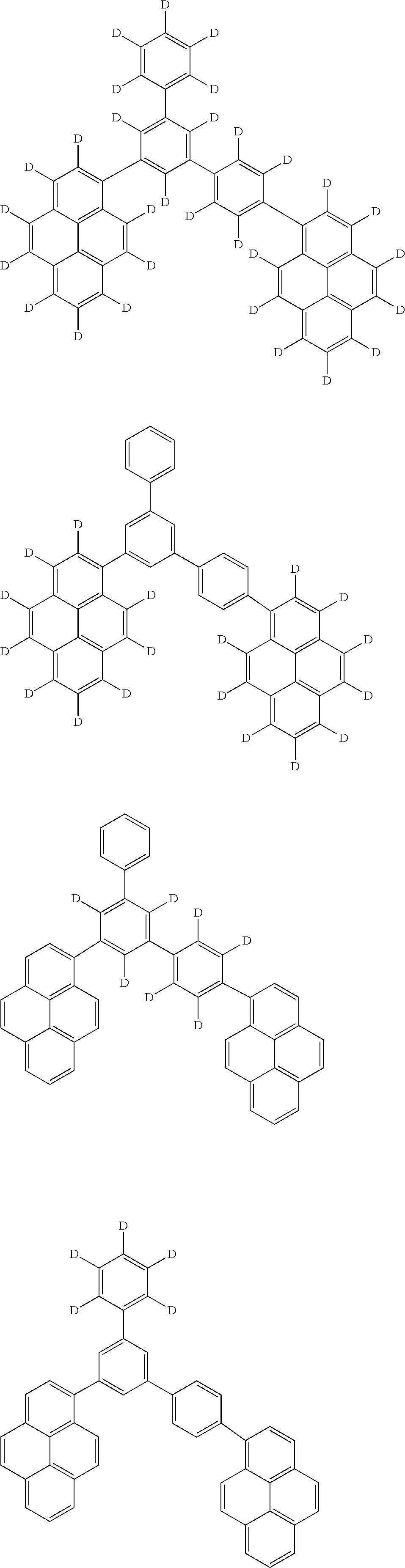

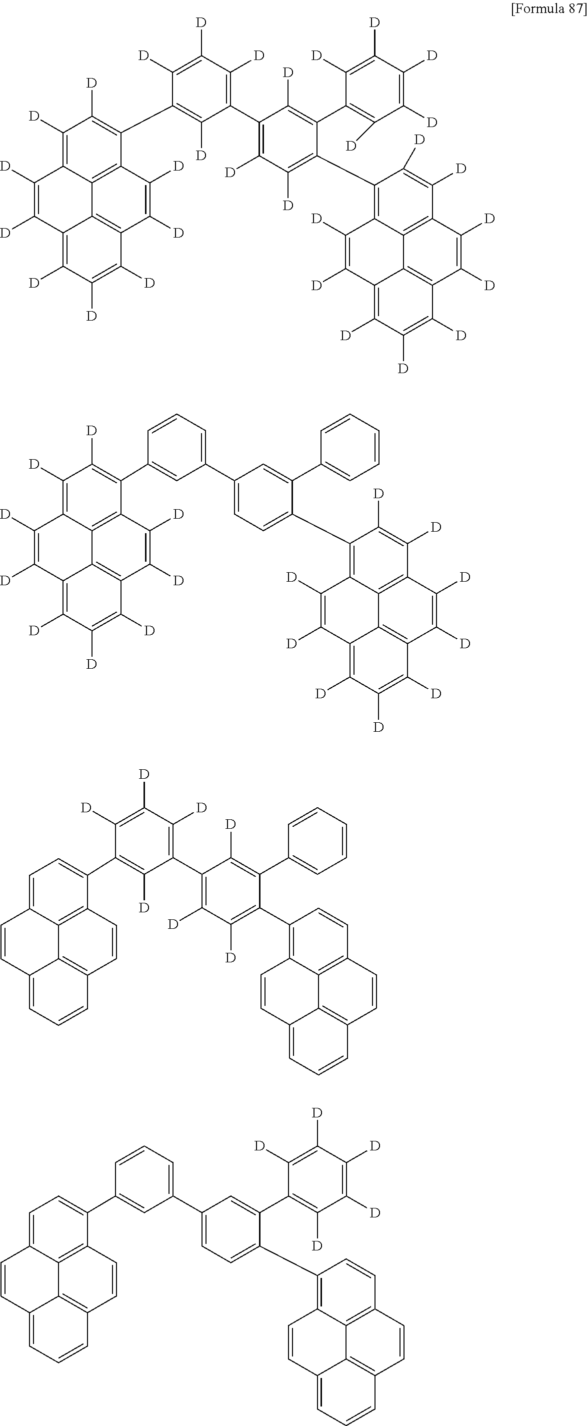

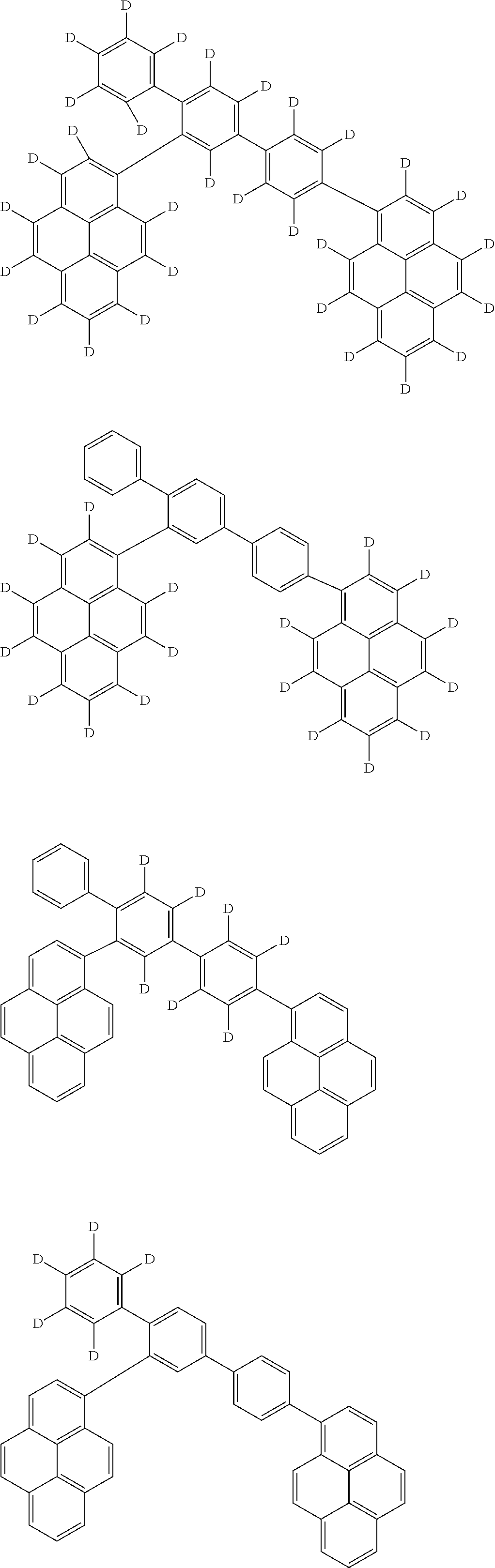

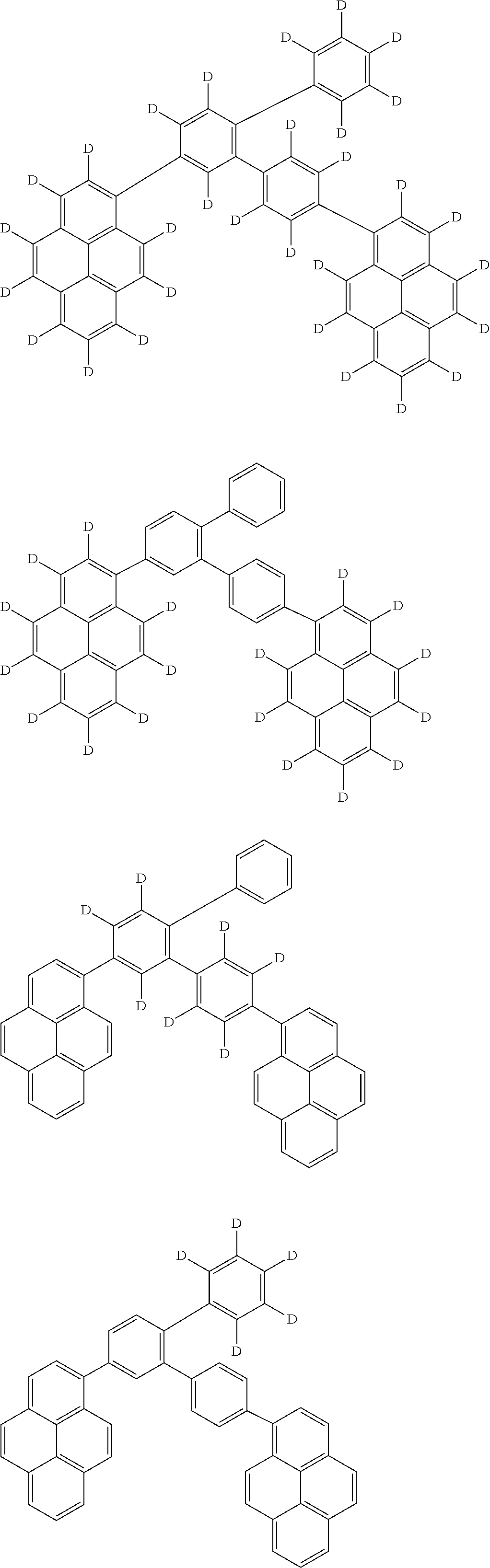

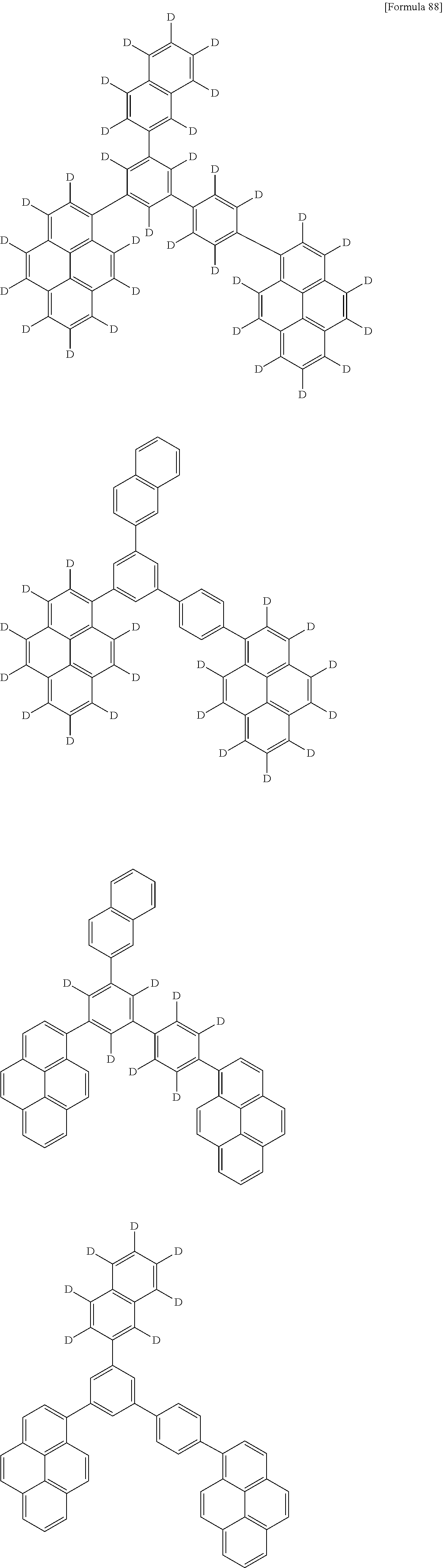

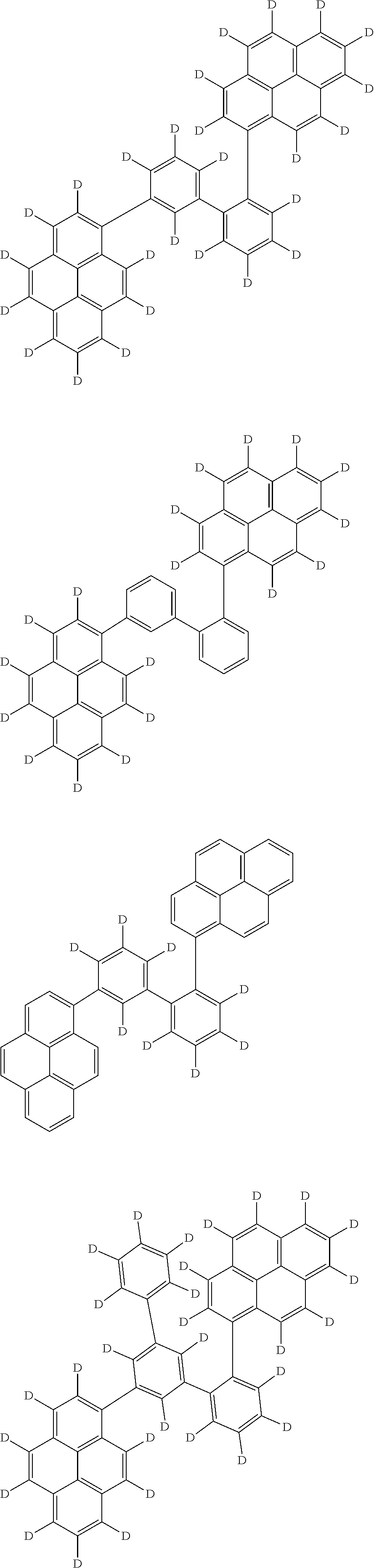

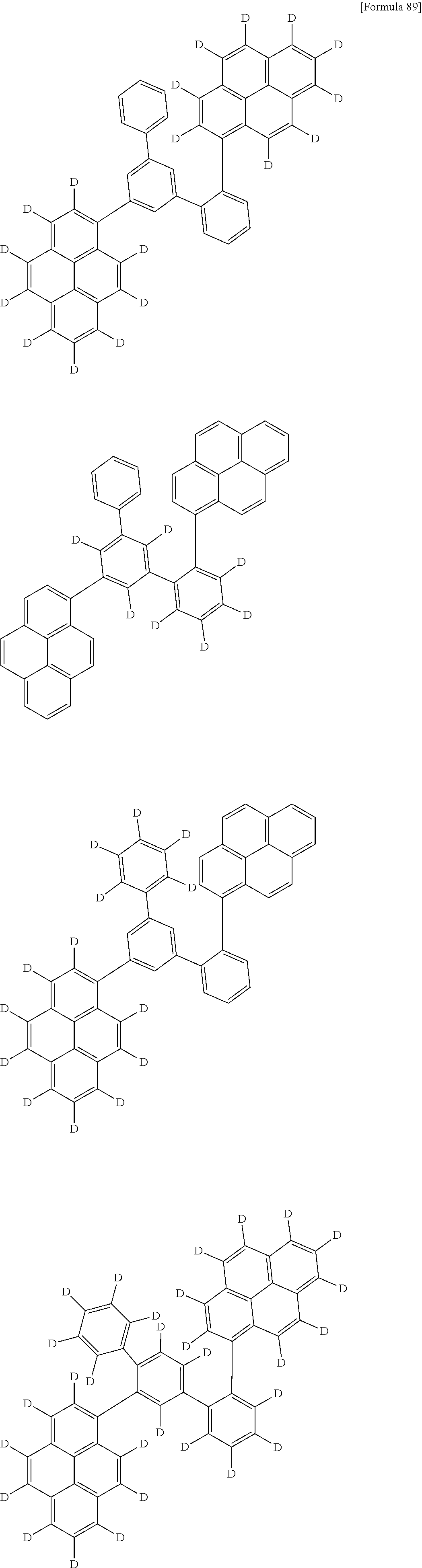

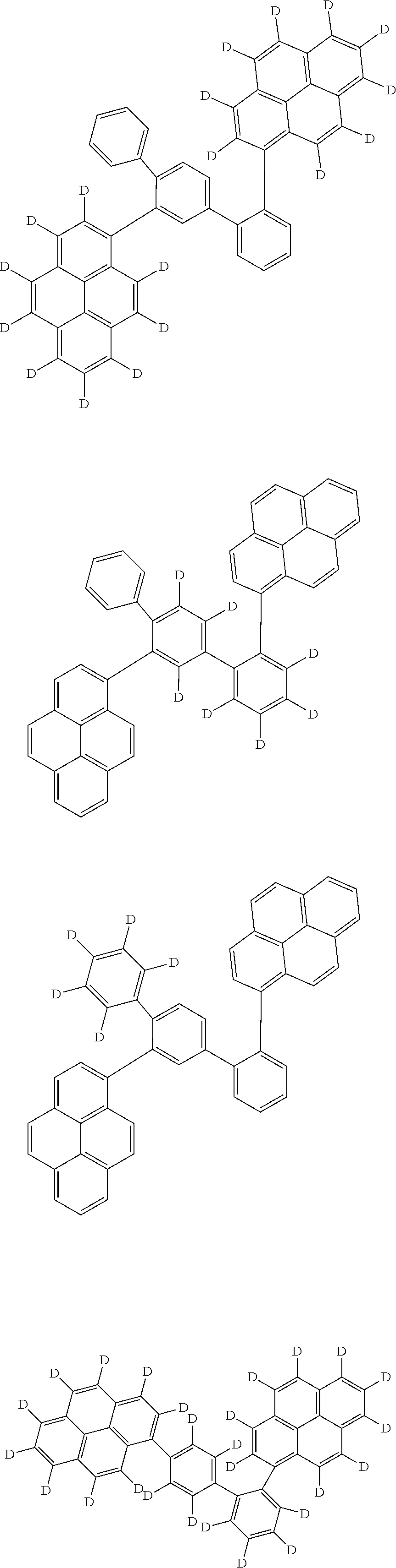

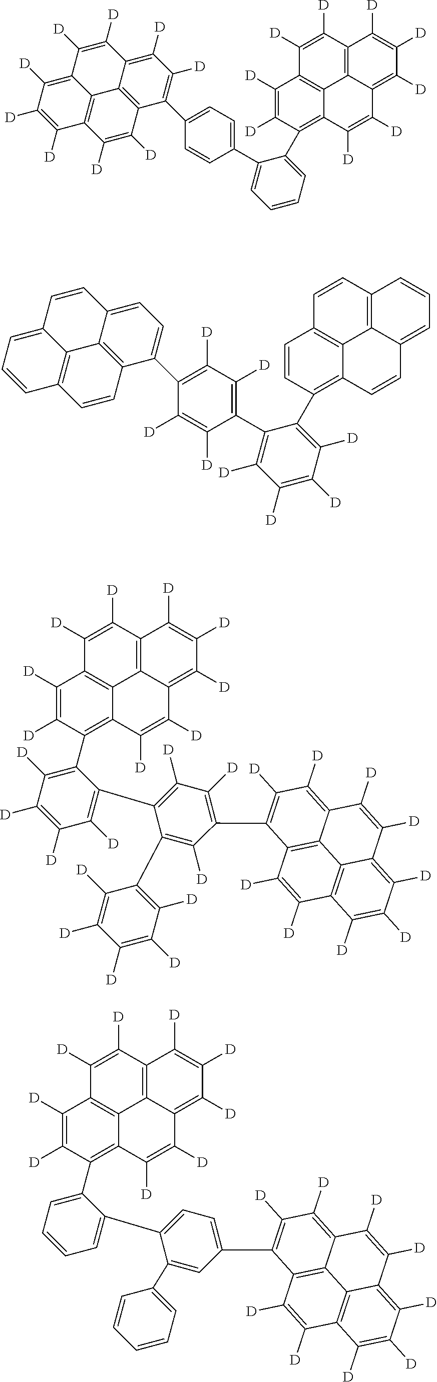

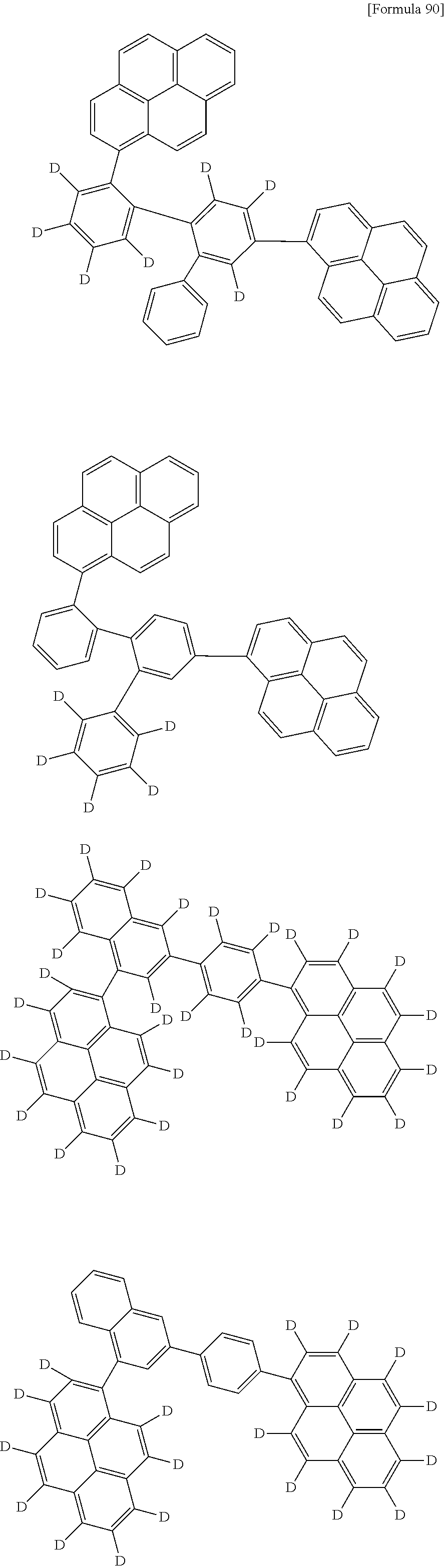

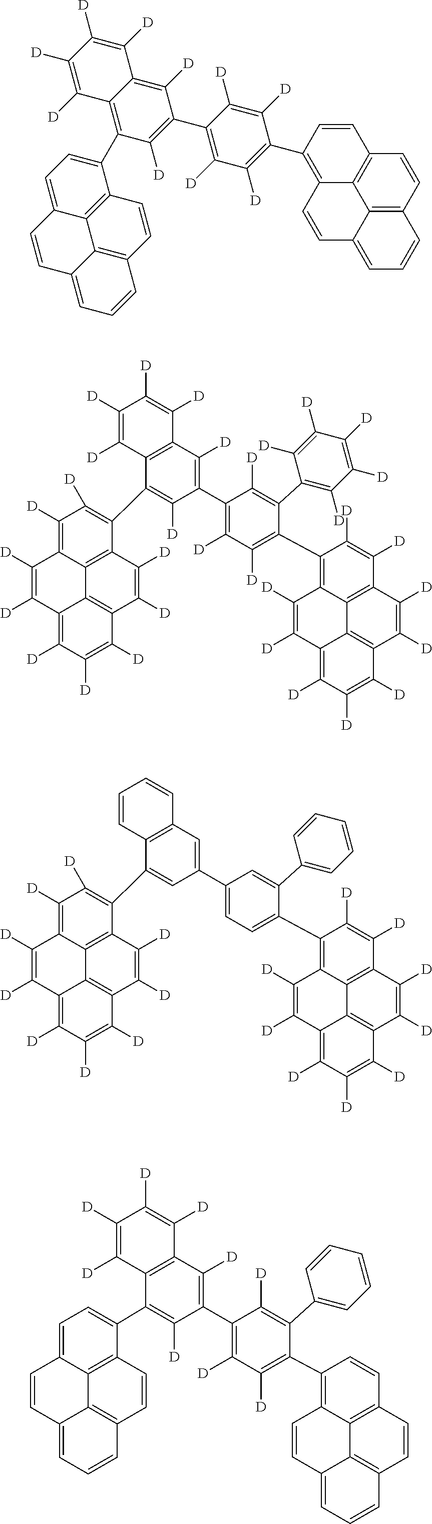

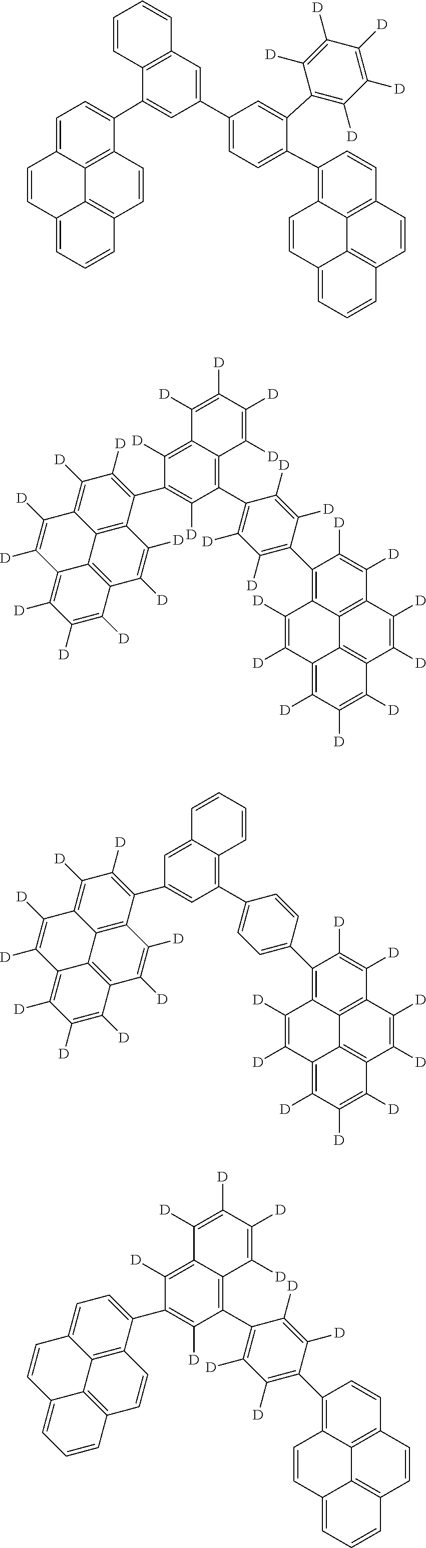

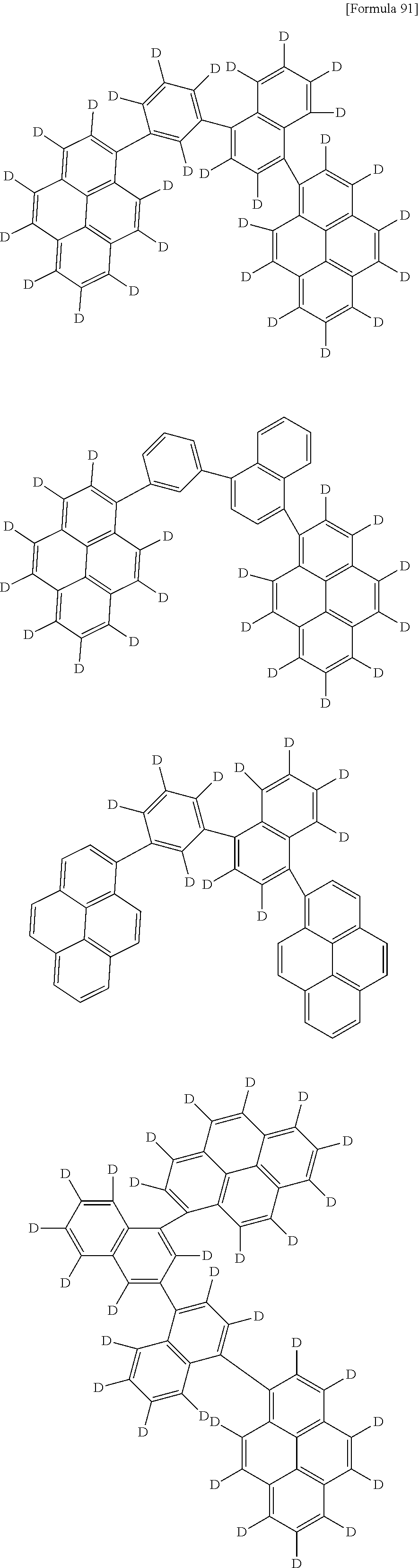

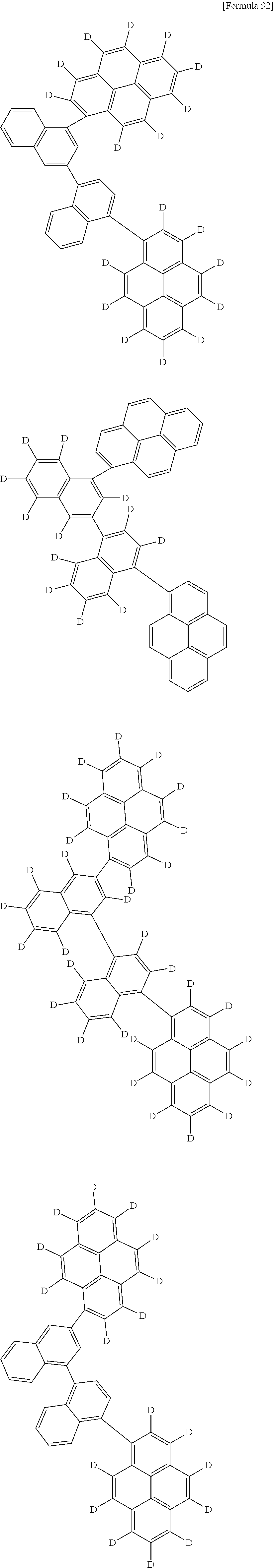

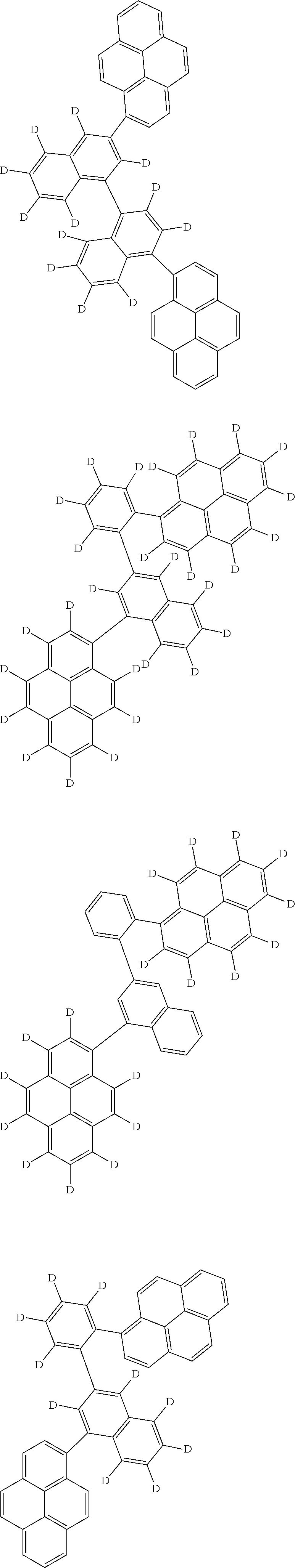

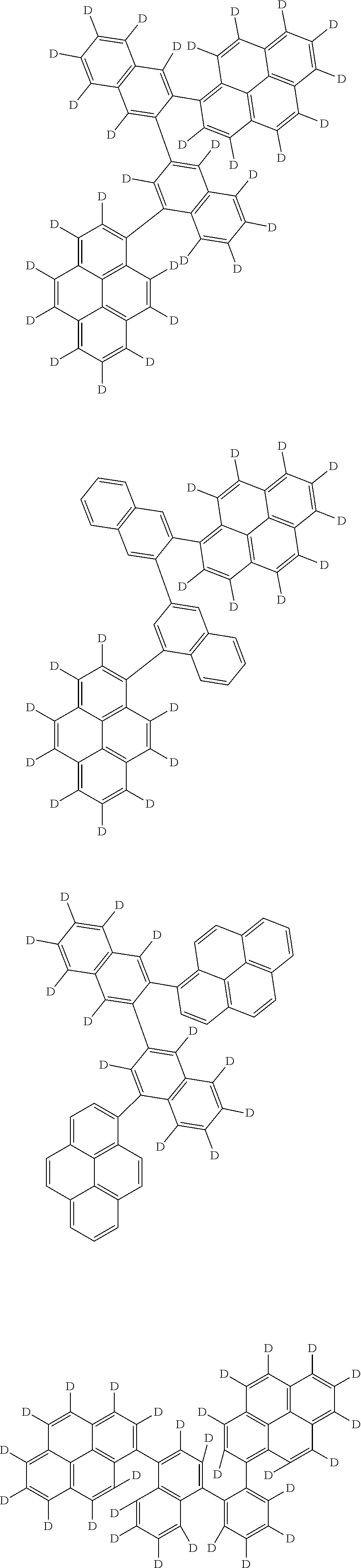

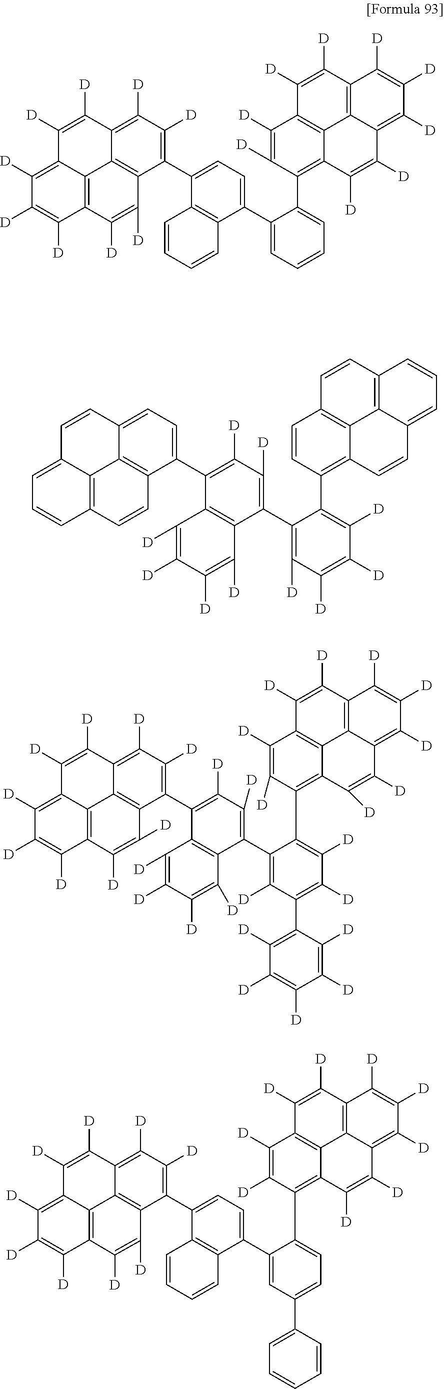

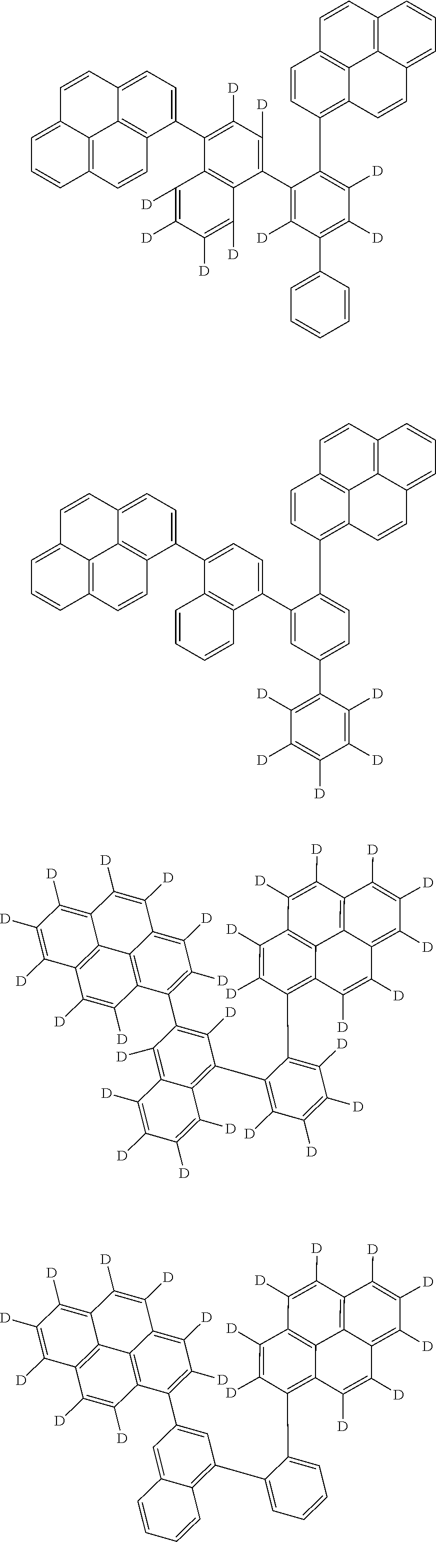

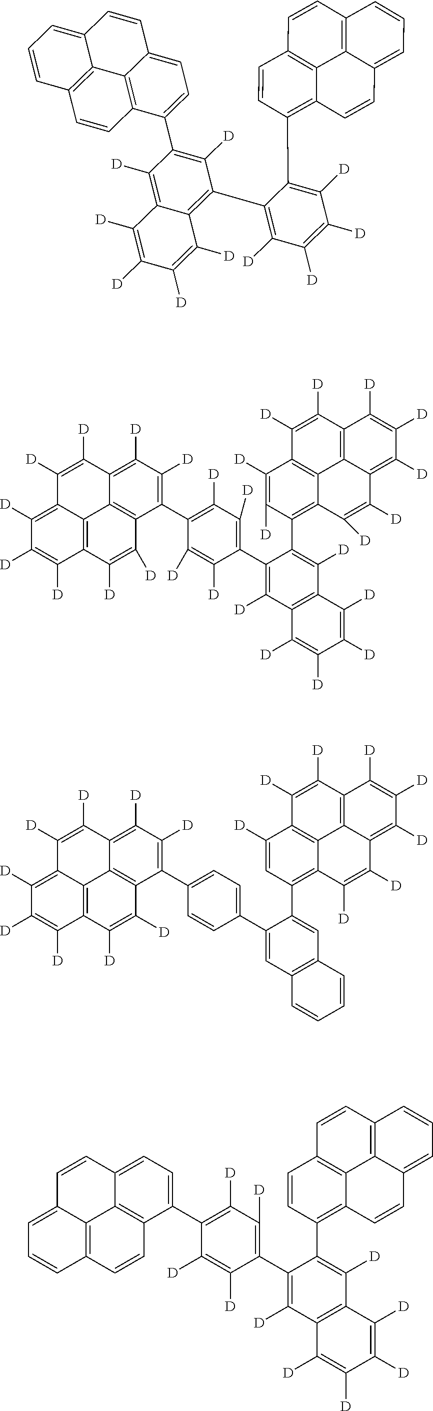

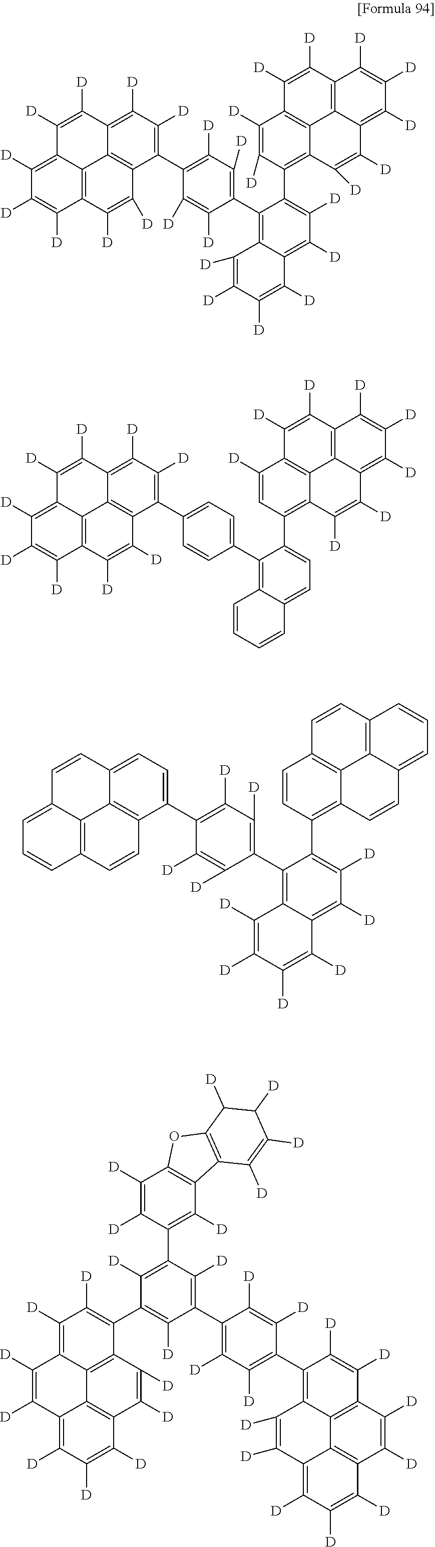

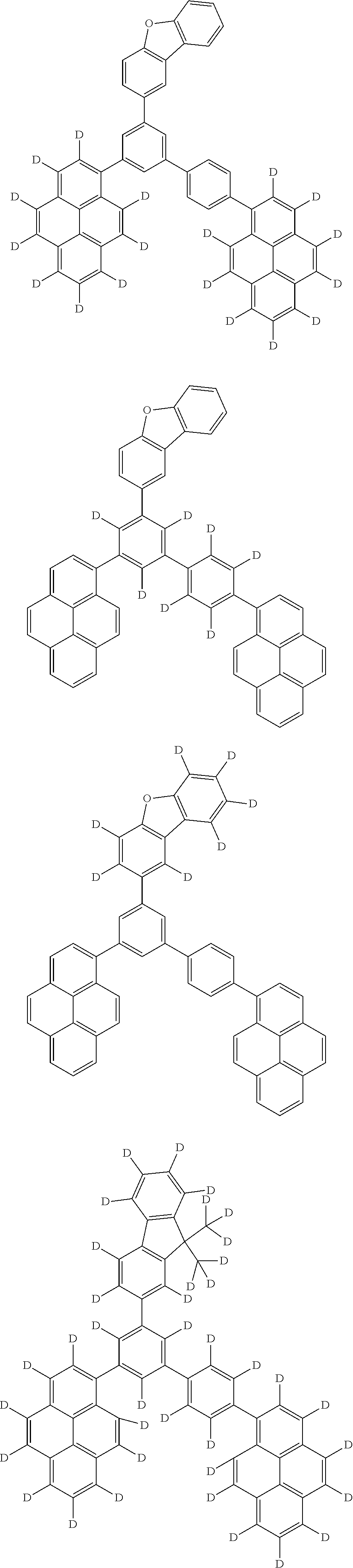

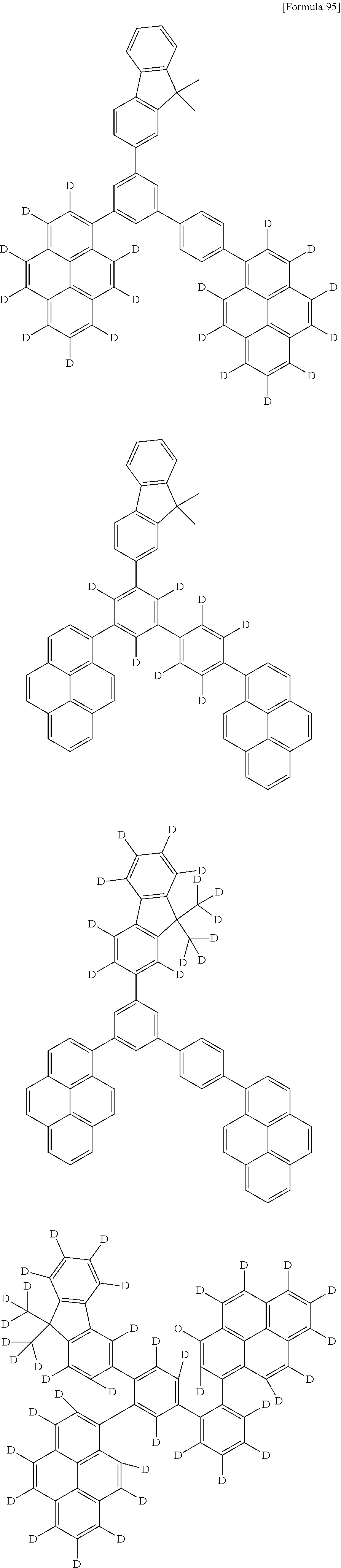

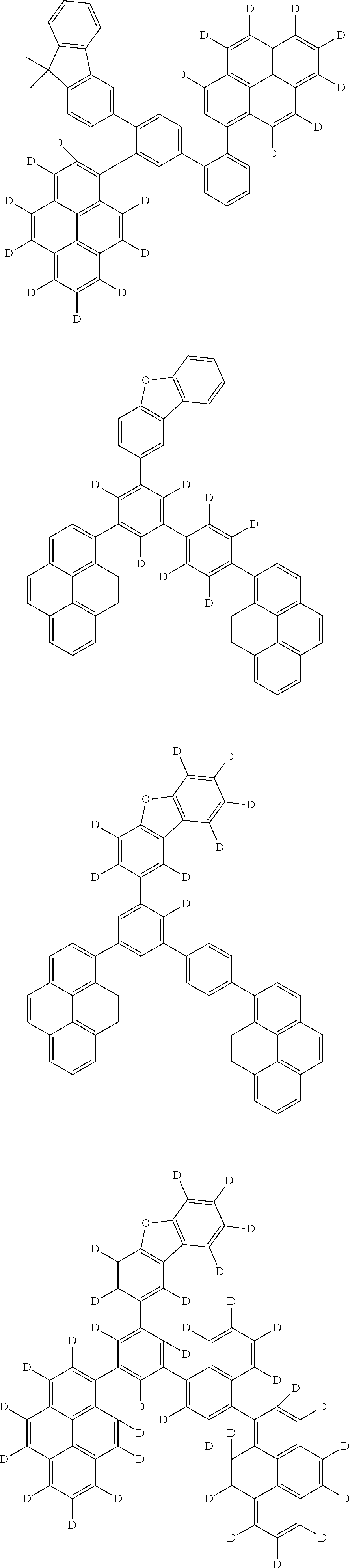

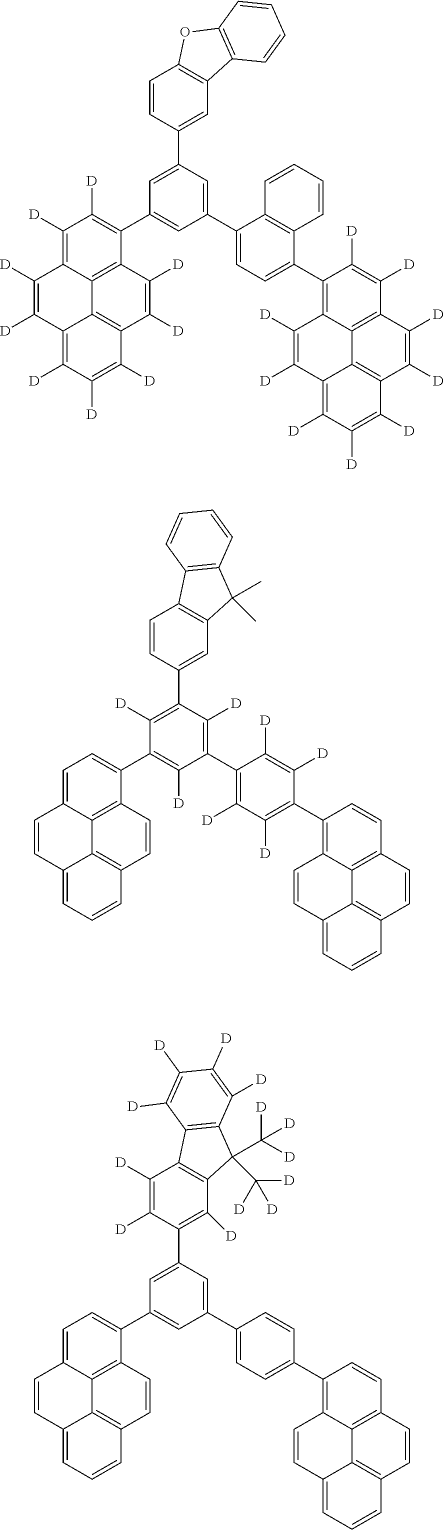

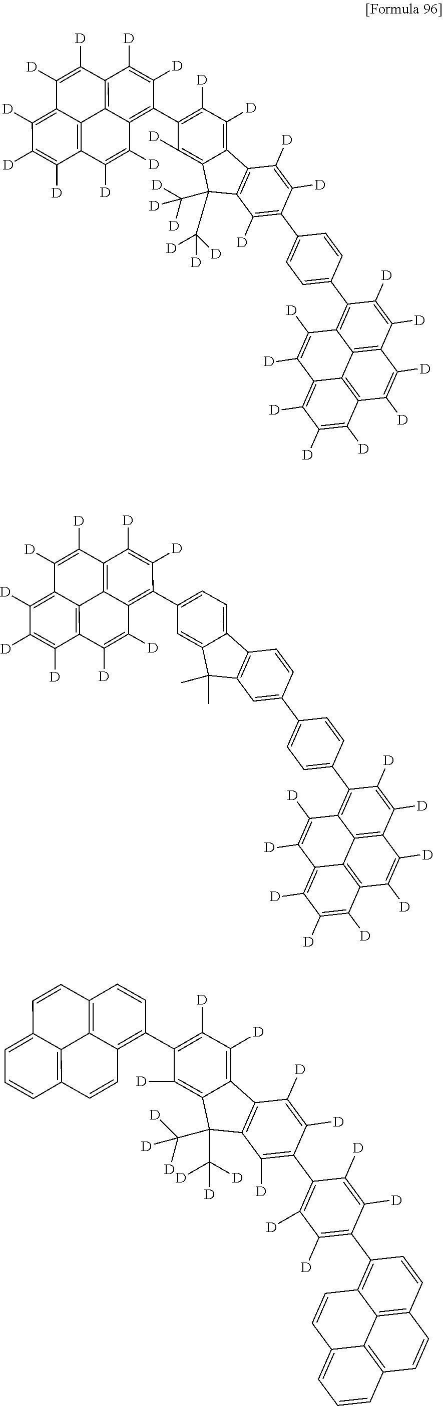

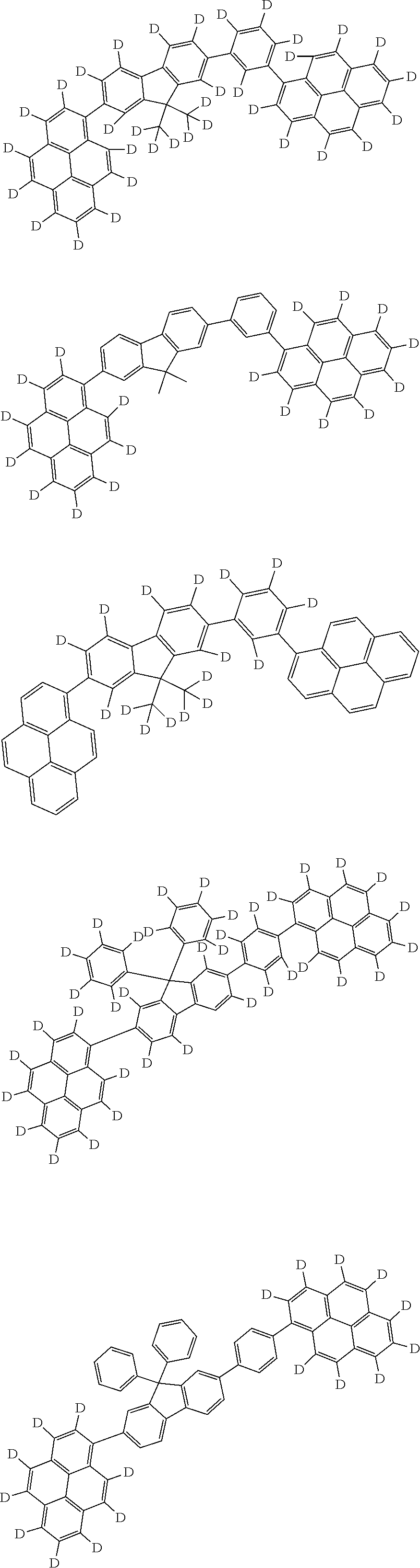

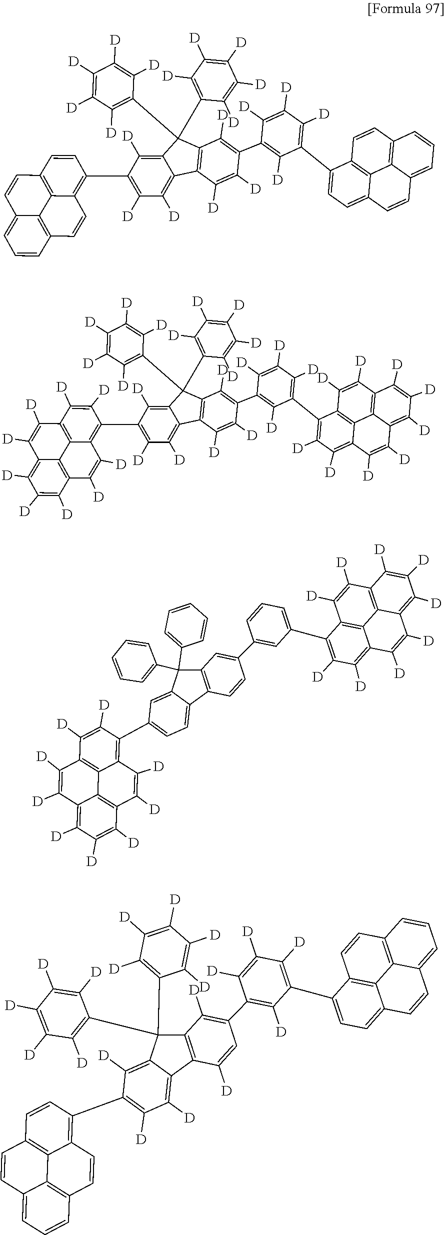

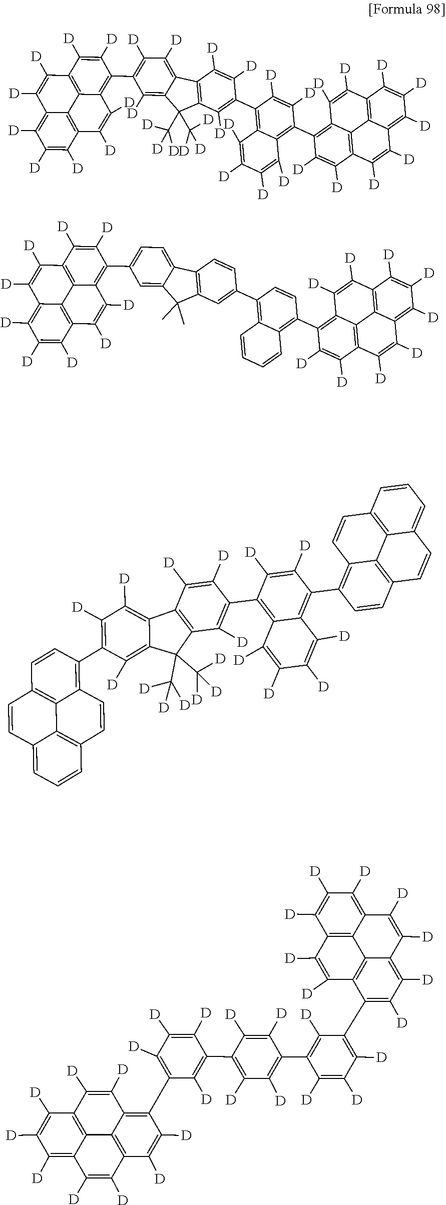

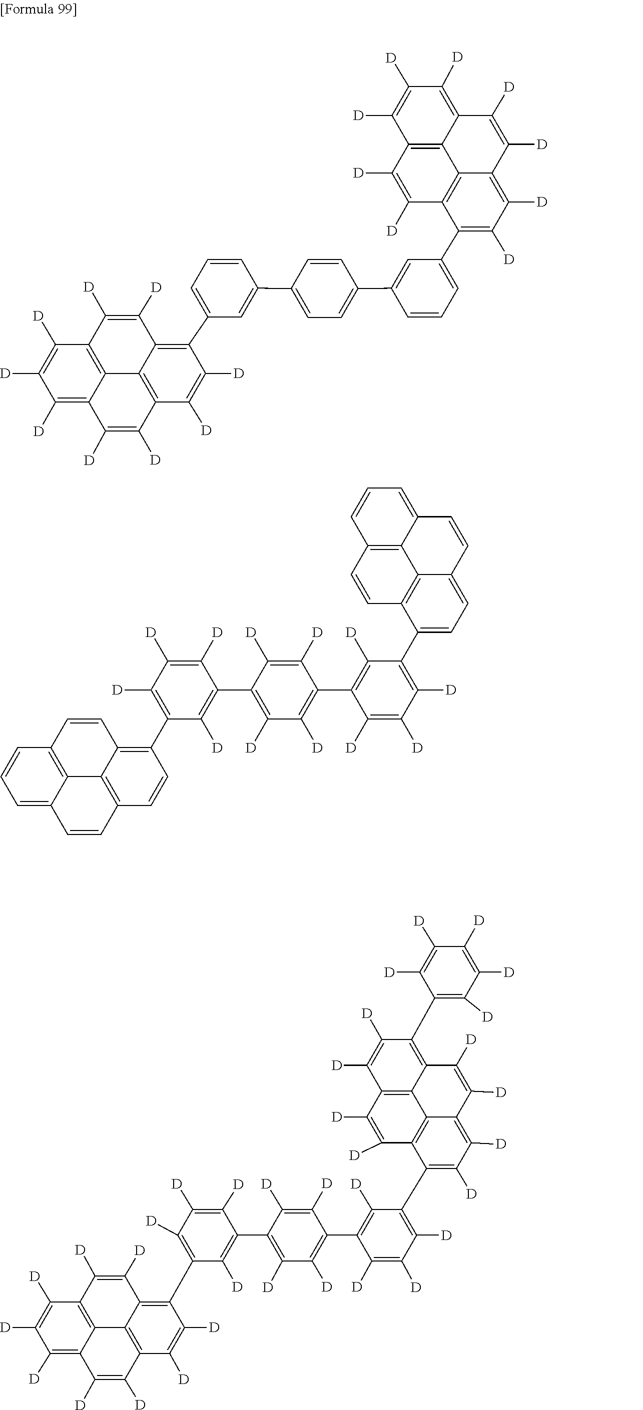

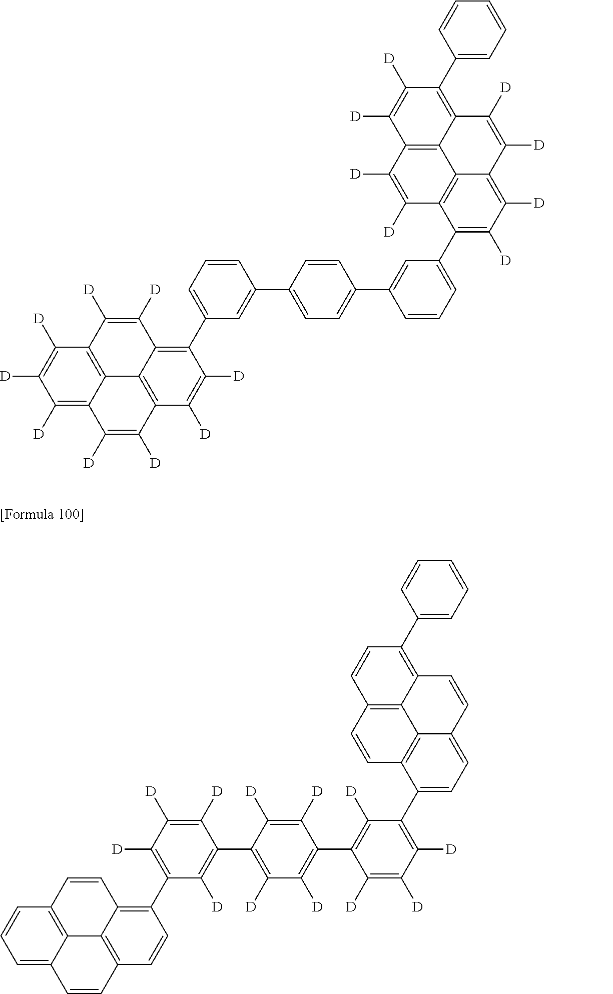 [SMILES: [2H]c1c([2H])c2c([2H])c([2H])c3c([2H])c([2H])c(-c4cccc(-c5ccc(-c6cccc(-c7c([2H])c([2H])c8c([2H])c([2H])c9c(-c%10ccccc%10)c([2H])c([2H])c%10c([2H])c([2H])c7c8c%109)c6)cc5)c4)c4c([2H])c([2H])c(c1[2H])c2c34 ; [2H]c1c([2H])c(-c2c([2H])c([2H])c(-c3c([2H])c([2H])c([2H])c(-c4ccc5ccc6c(-c7ccccc7)ccc7ccc4c5c76)c3[2H])c([2H])c2[2H])c([2H])c(-c2ccc3ccc4cccc5ccc2c3c45)c1[2H]]

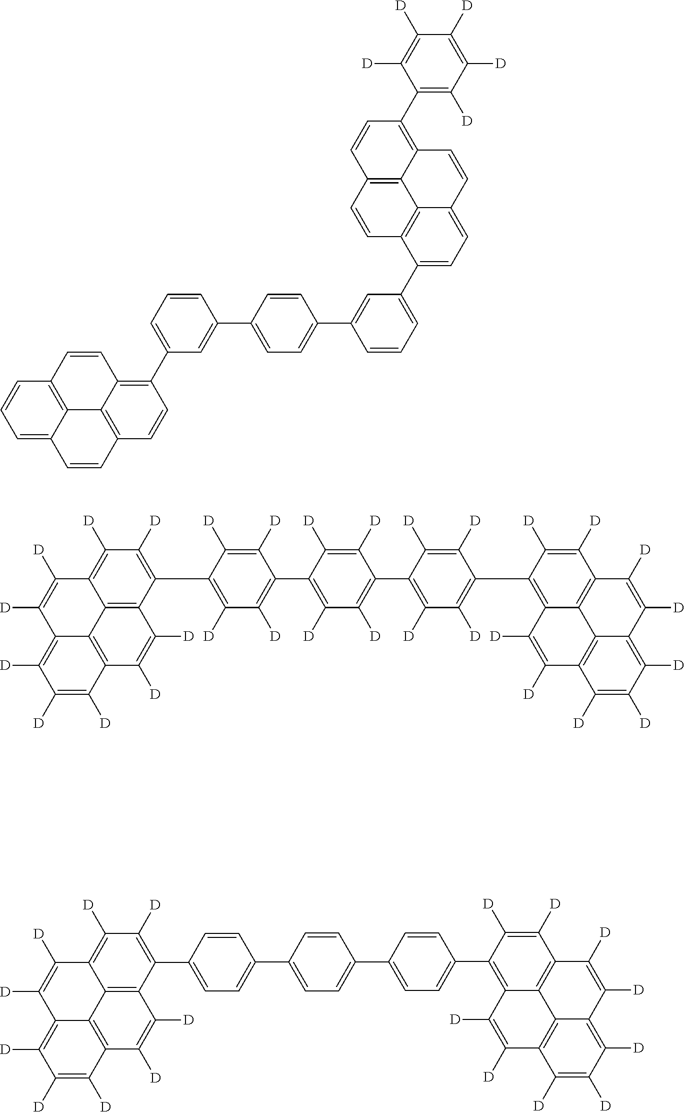

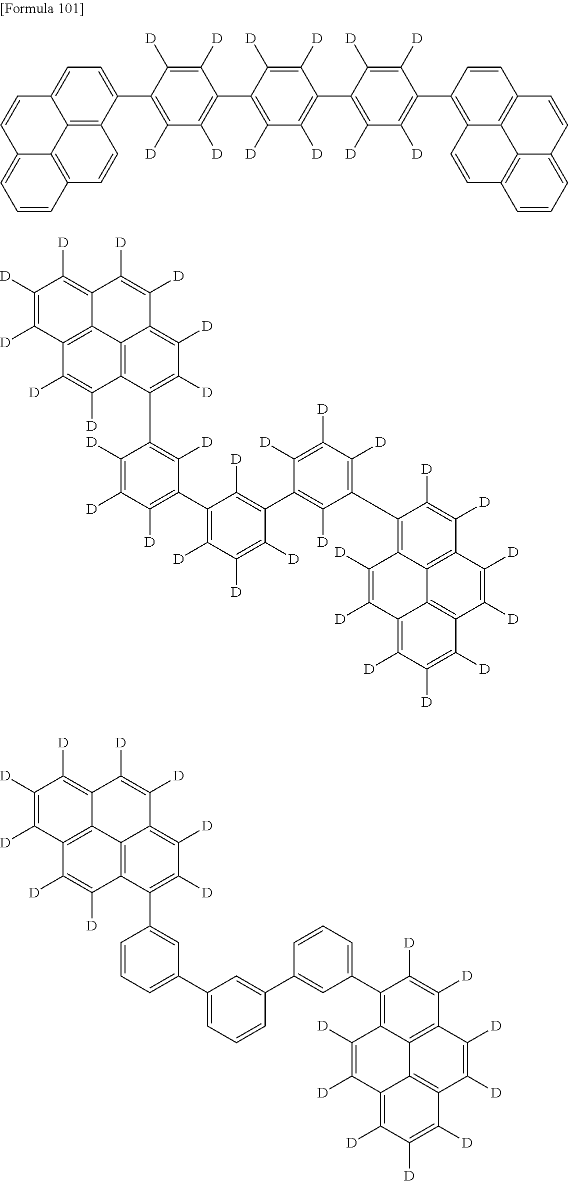

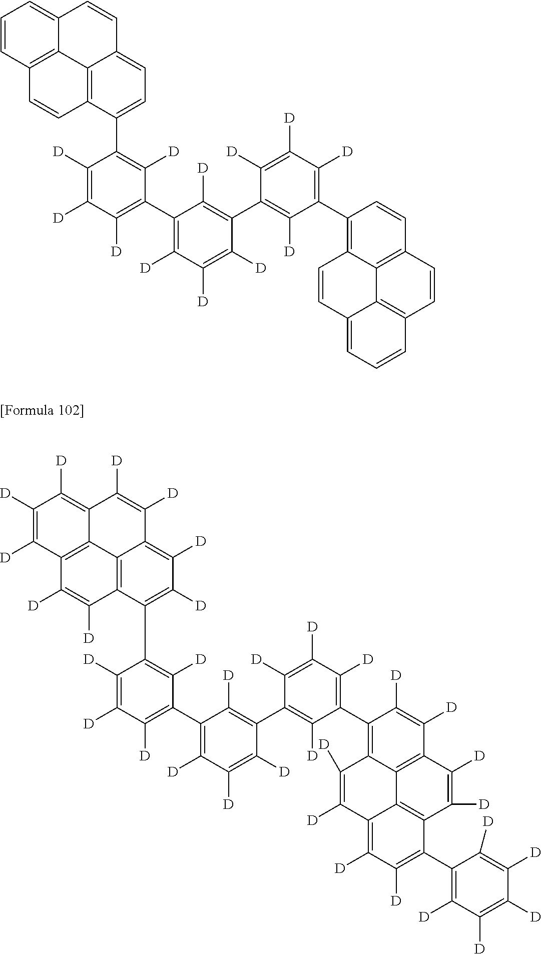 [SMILES: [2H]c1c([2H])c(-c2c([2H])c([2H])c([2H])c(-c3ccc4ccc5cccc6ccc3c4c56)c2[2H])c([2H])c(-c2c([2H])c([2H])c([2H])c(-c3ccc4ccc5cccc6ccc3c4c56)c2[2H])c1[2H] ; [2H]c1c([2H])c([2H])c(-c2c([2H])c([2H])c3c([2H])c([2H])c4c(-c5c([2H])c([2H])c([2H])c(-c6c([2H])c([2H])c([2H])c(-c7c([2H])c([2H])c([2H])c(-c8c([2H])c([2H])c9c([2H])c([2H])c%10c([2H])c([2H])c([2H])c%11c([2H])c([2H])c8c9c%10%11)c7[2H])c6[2H])c5[2H])c([2H])c([2H])c5c([2H])c([2H])c2c3c54)c([2H])c1[2H]]

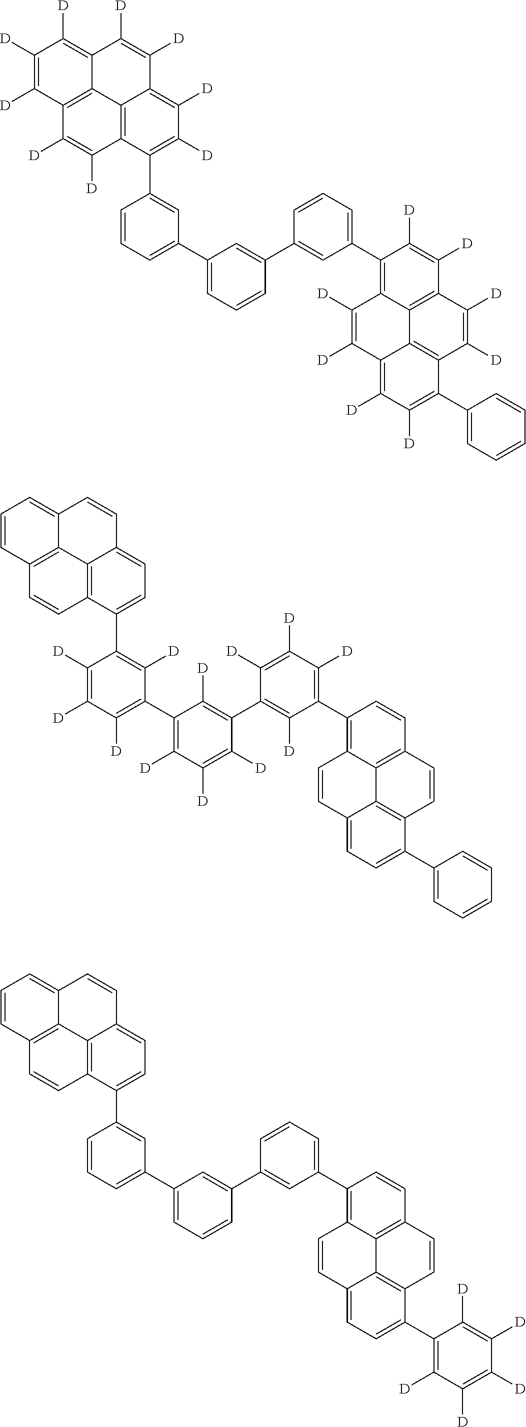 [SMILES: [2H]c1c([2H])c2c([2H])c([2H])c3c([2H])c([2H])c(-c4cccc(-c5cccc(-c6cccc(-c7c([2H])c([2H])c8c([2H])c([2H])c9c(-c%10ccccc%10)c([2H])c([2H])c%10c([2H])c([2H])c7c8c%109)c6)c5)c4)c4c([2H])c([2H])c(c1[2H])c2c34 ; [2H]c1c([2H])c(-c2c([2H])c([2H])c([2H])c(-c3ccc4ccc5cccc6ccc3c4c56)c2[2H])c([2H])c(-c2c([2H])c([2H])c([2H])c(-c3ccc4ccc5c(-c6ccccc6)ccc6ccc3c4c65)c2[2H])c1[2H] ; [2H]c1c([2H])c([2H])c(-c2ccc3ccc4c(-c5cccc(-c6cccc(-c7cccc(-c8ccc9ccc%10cccc%11ccc8c9c%10%11)c7)c6)c5)ccc5ccc2c3c54)c([2H])c1[2H]]

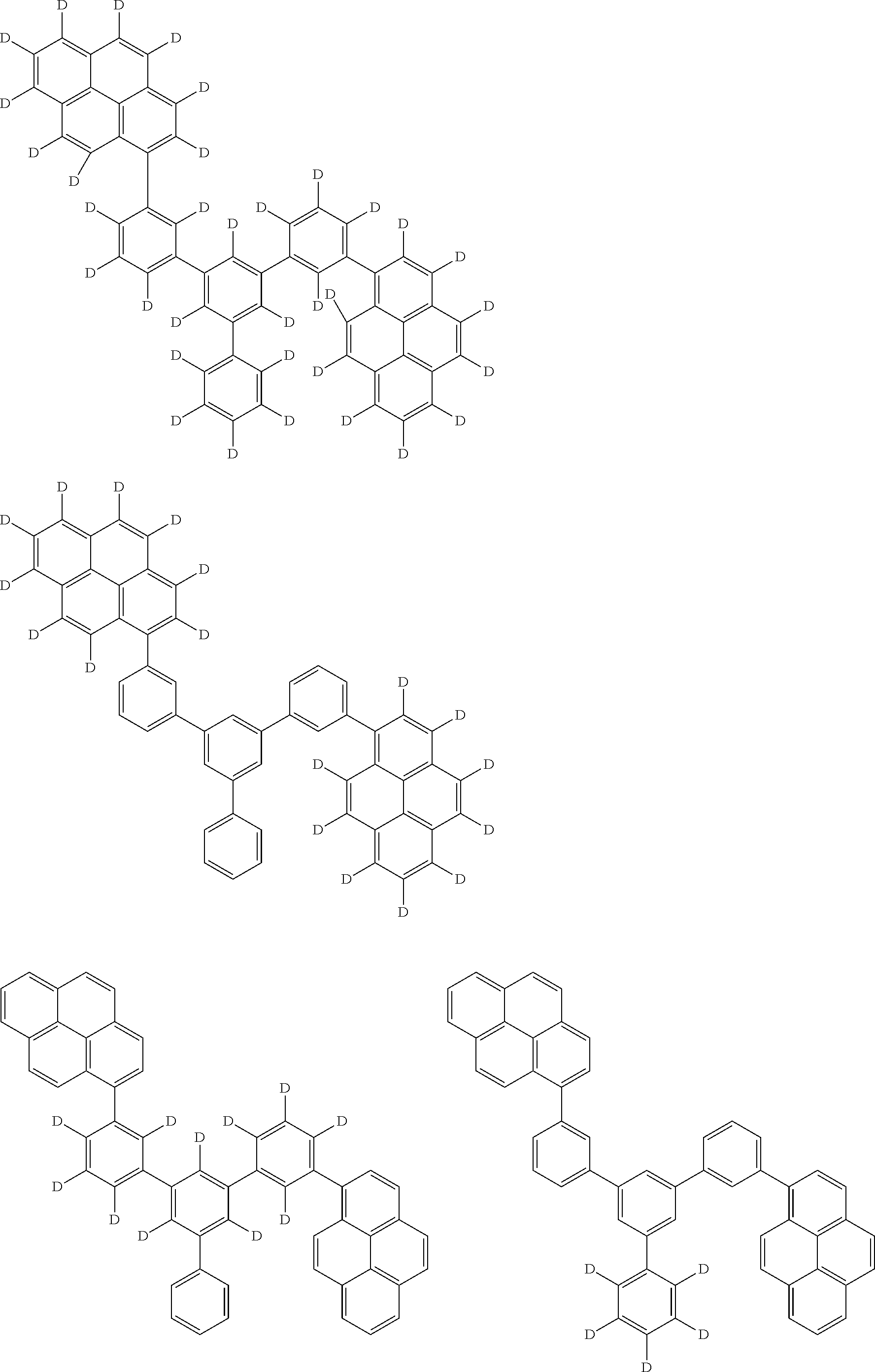

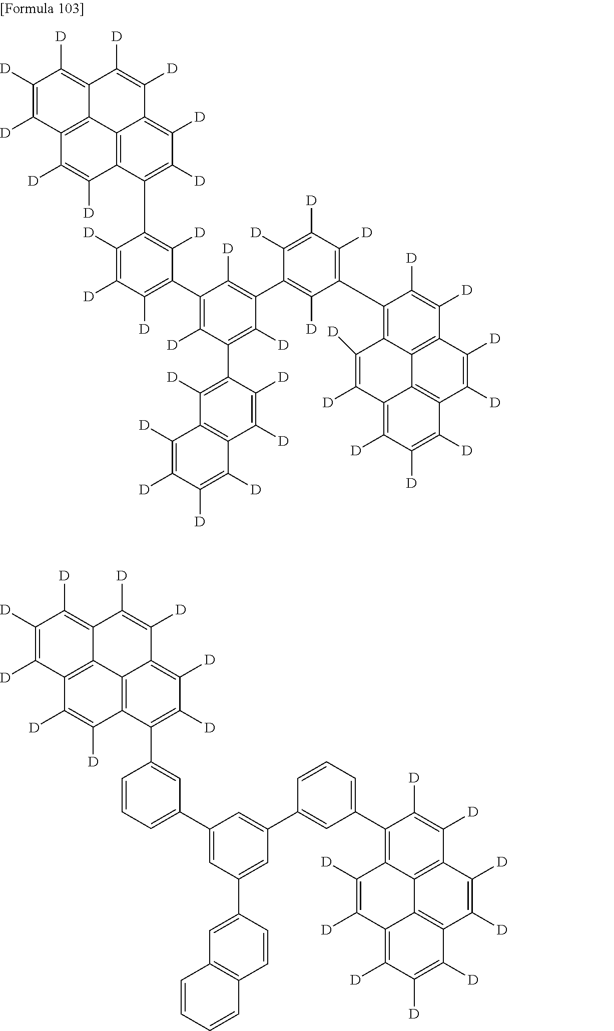

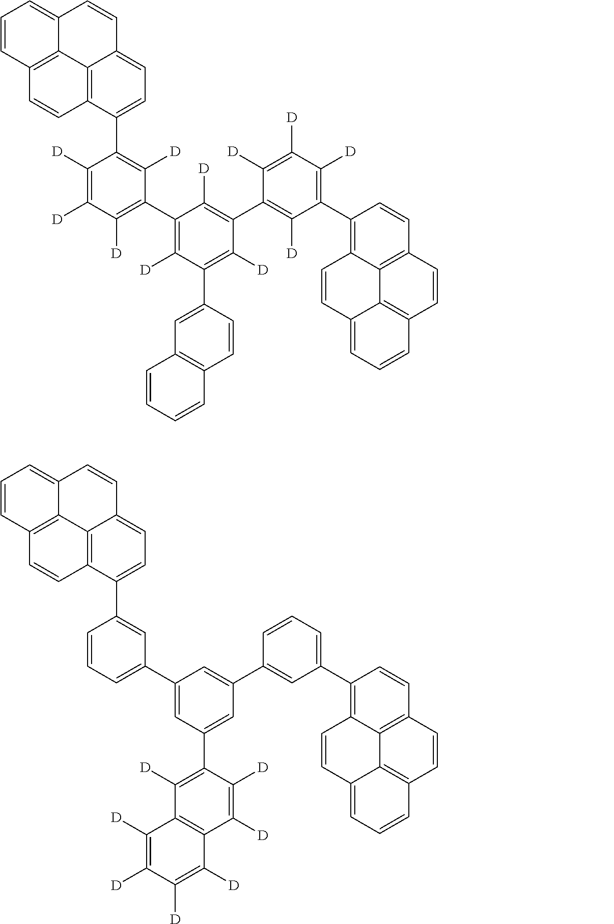 [SMILES: [2H]c1c([2H])c(-c2c([2H])c(-c3ccc4ccccc4c3)c([2H])c(-c3c([2H])c([2H])c([2H])c(-c4ccc5ccc6cccc7ccc4c5c67)c3[2H])c2[2H])c([2H])c(-c2ccc3ccc4cccc5ccc2c3c45)c1[2H] ; [2H]c1c([2H])c([2H])c2c([2H])c(-c3cc(-c4cccc(-c5ccc6ccc7cccc8ccc5c6c78)c4)cc(-c4cccc(-c5ccc6ccc7cccc8ccc5c6c78)c4)c3)c([2H])c([2H])c2c1[2H]]

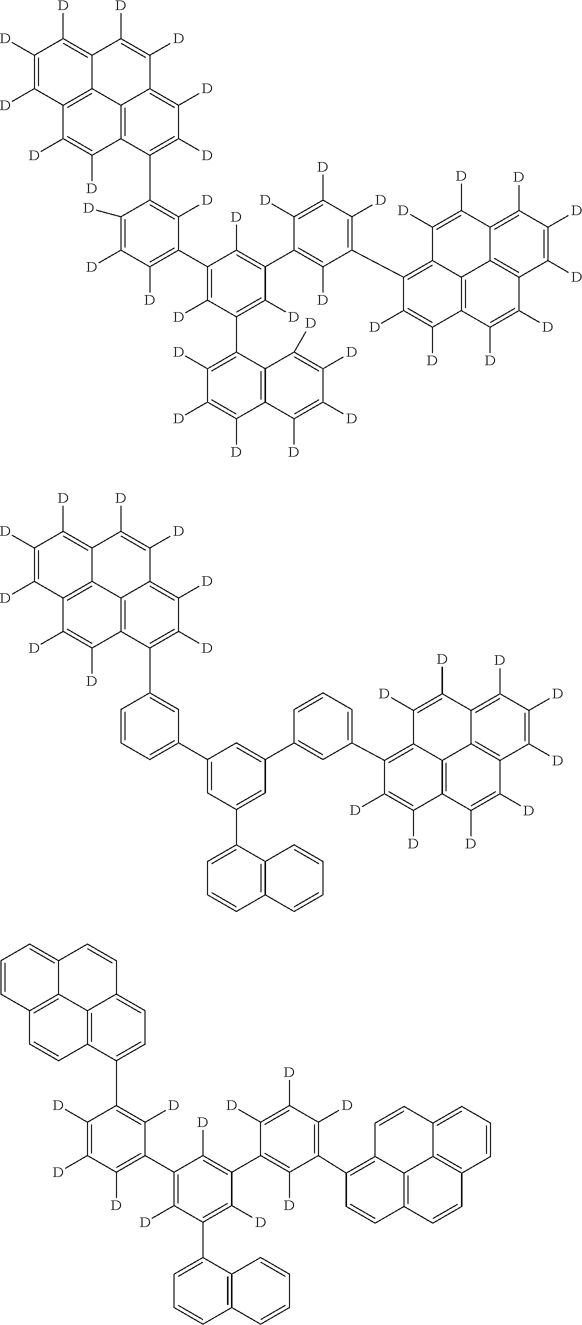

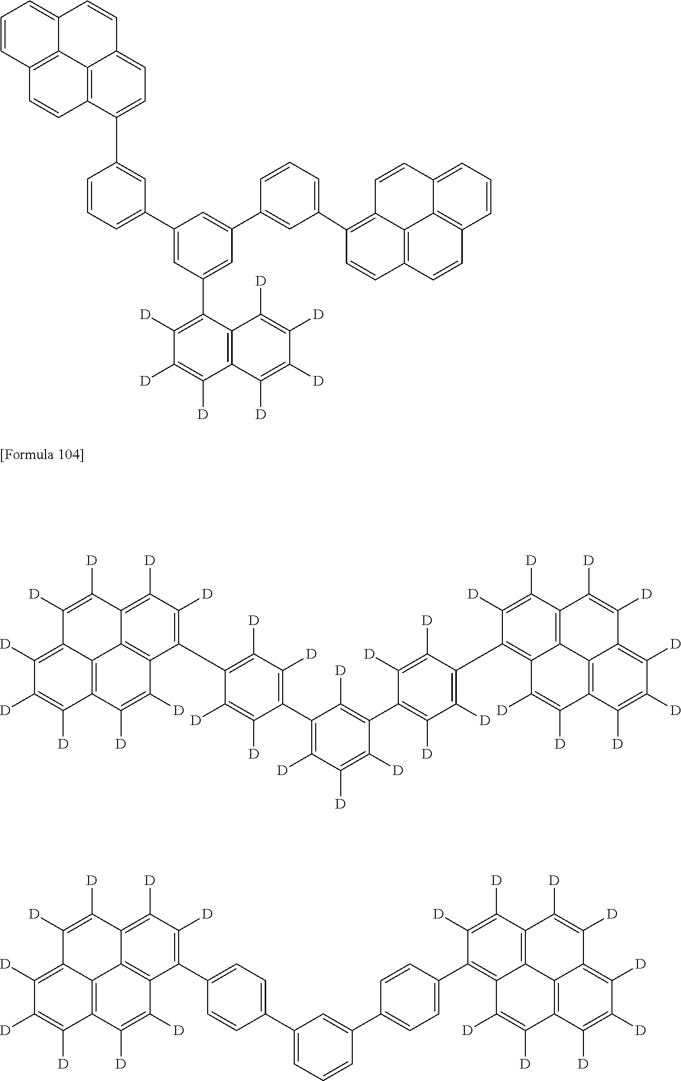

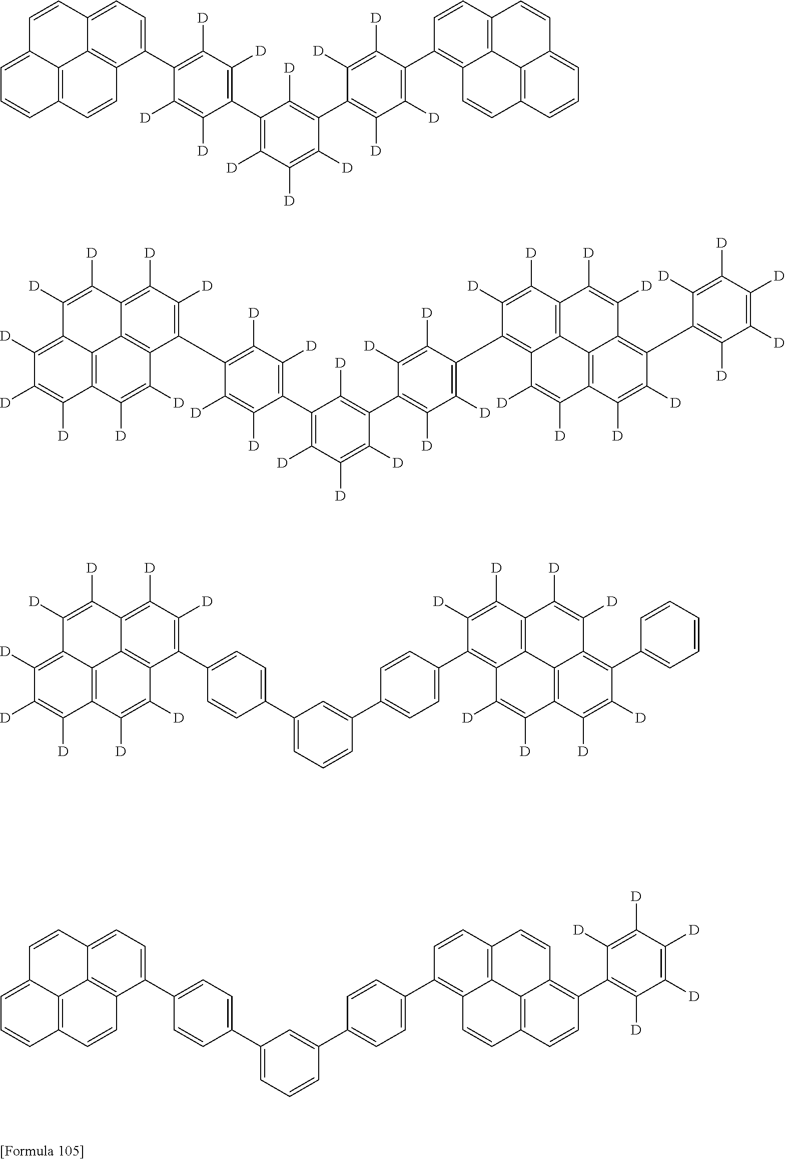

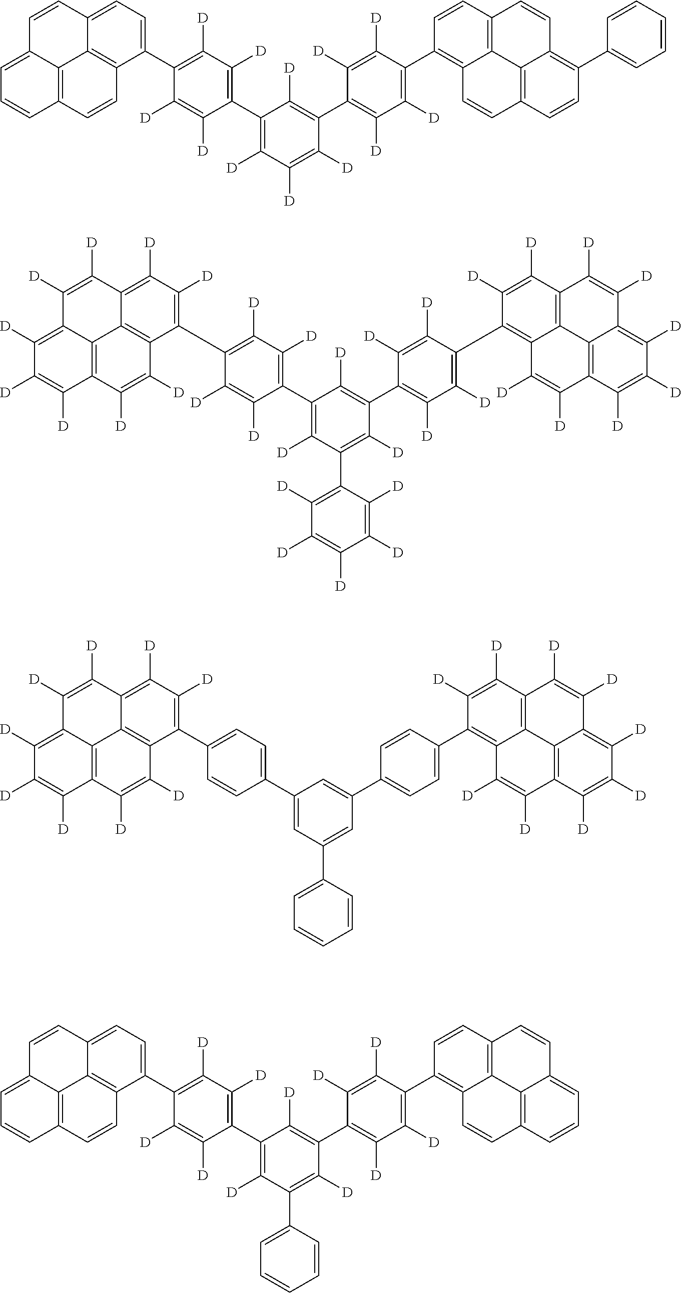

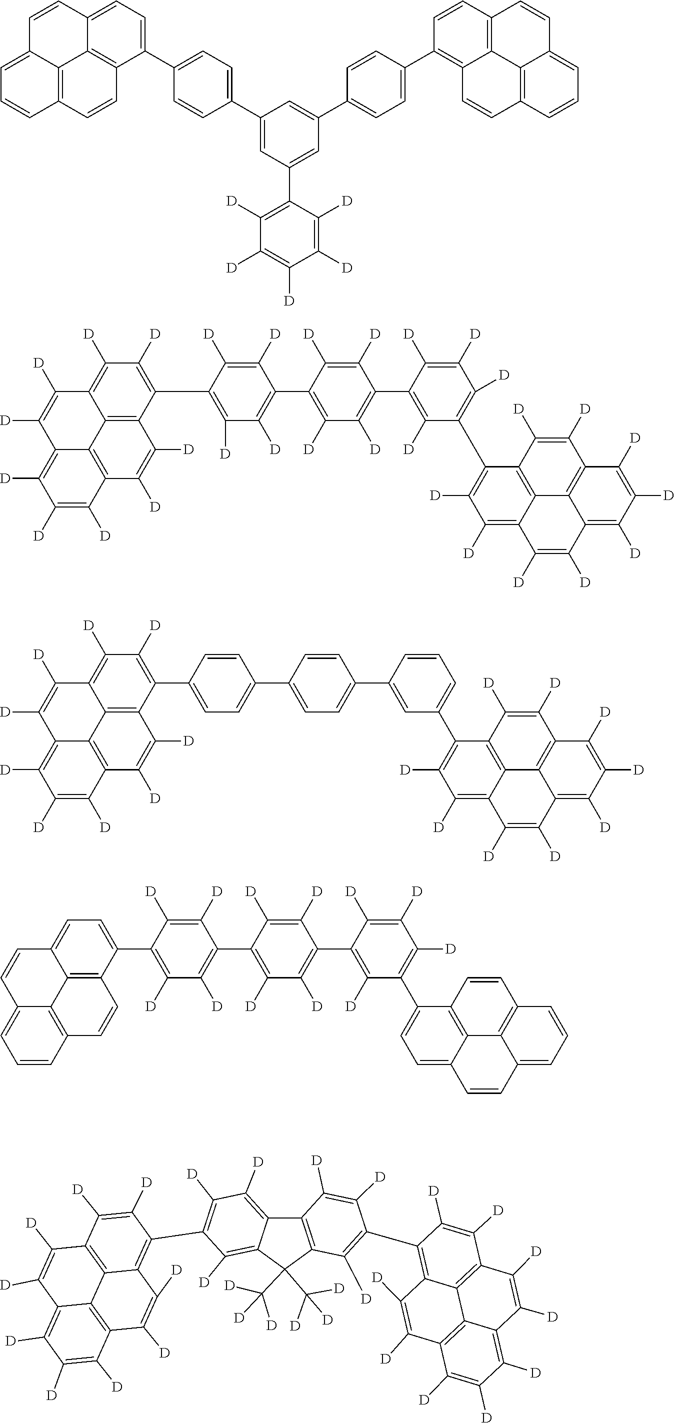

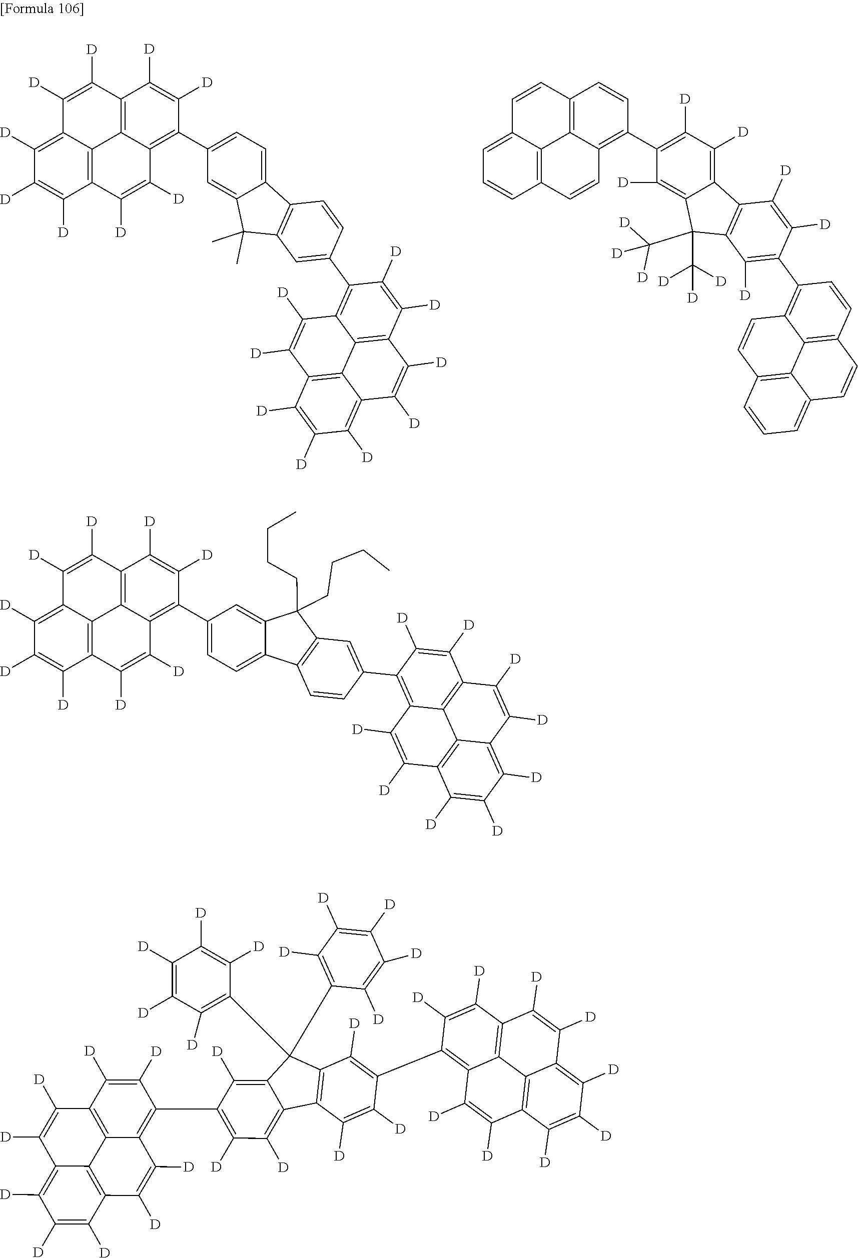

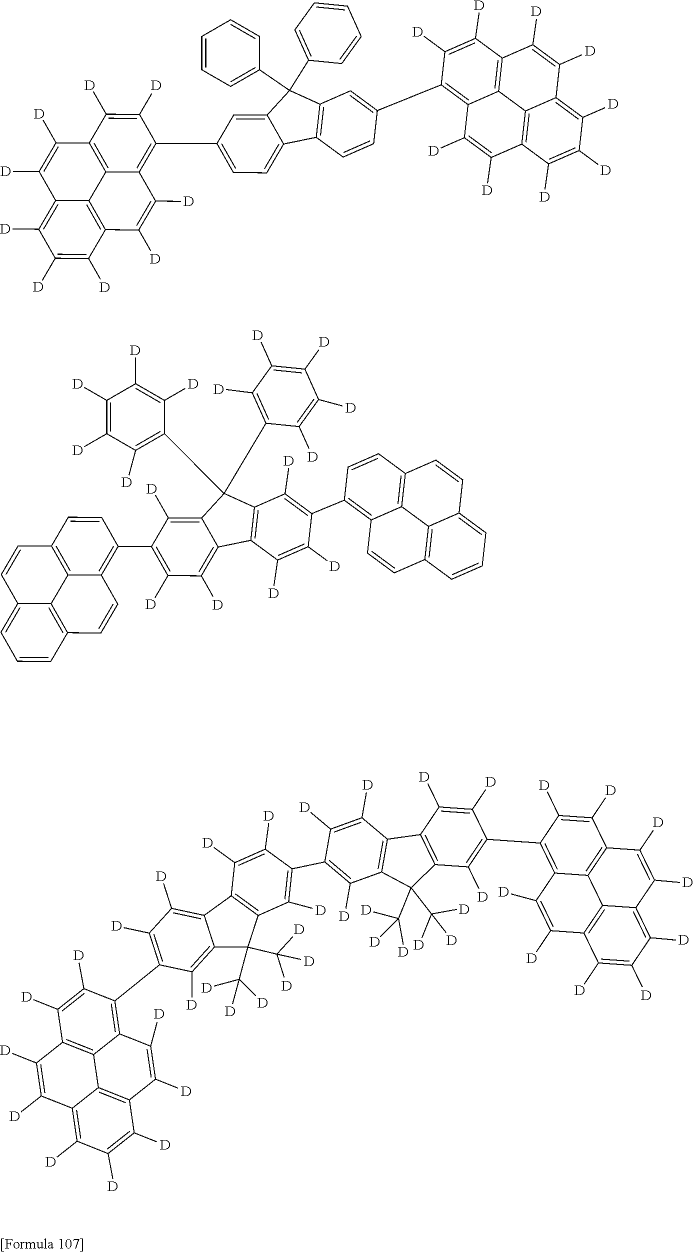

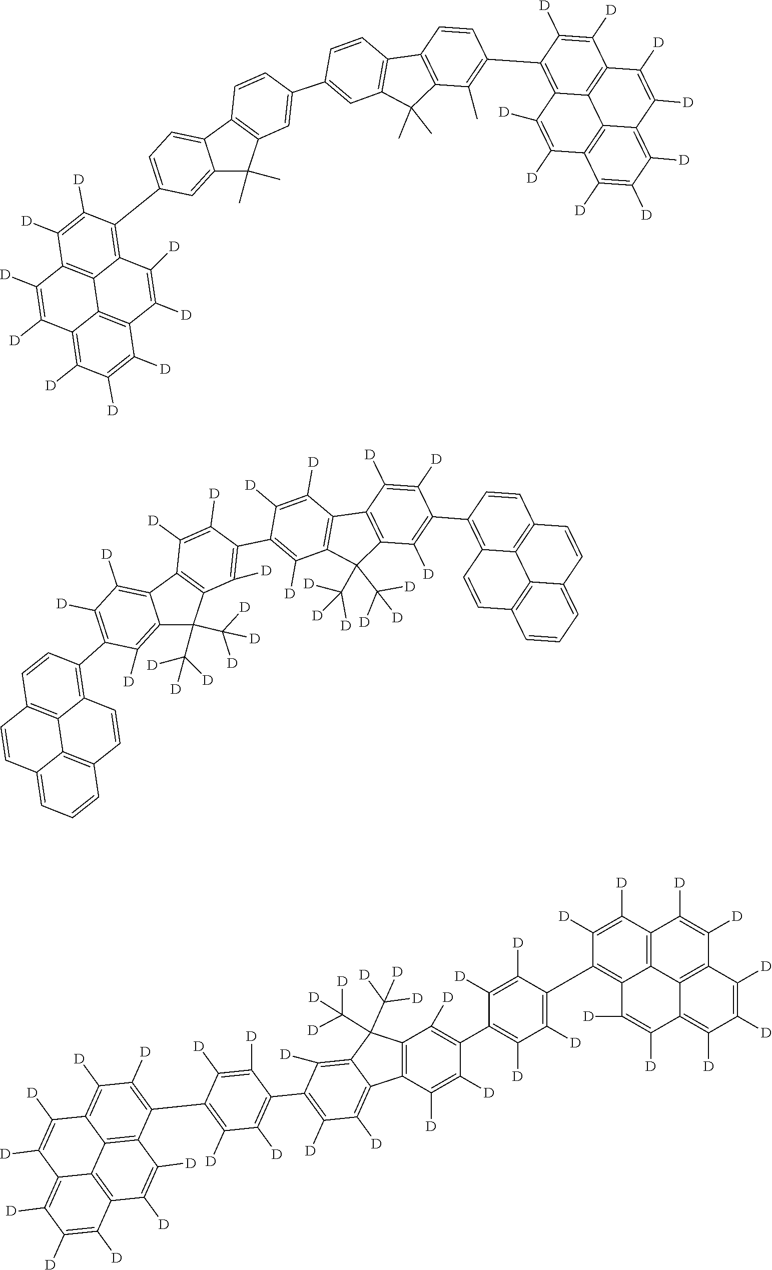

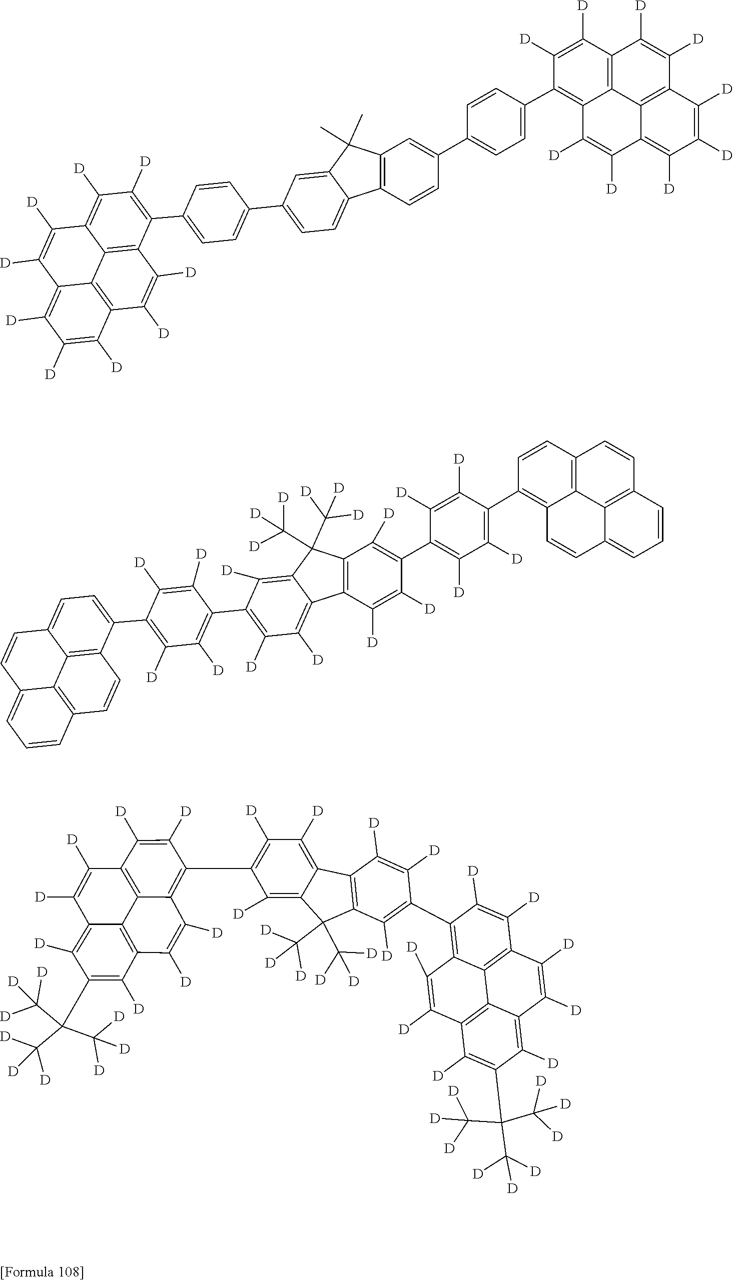

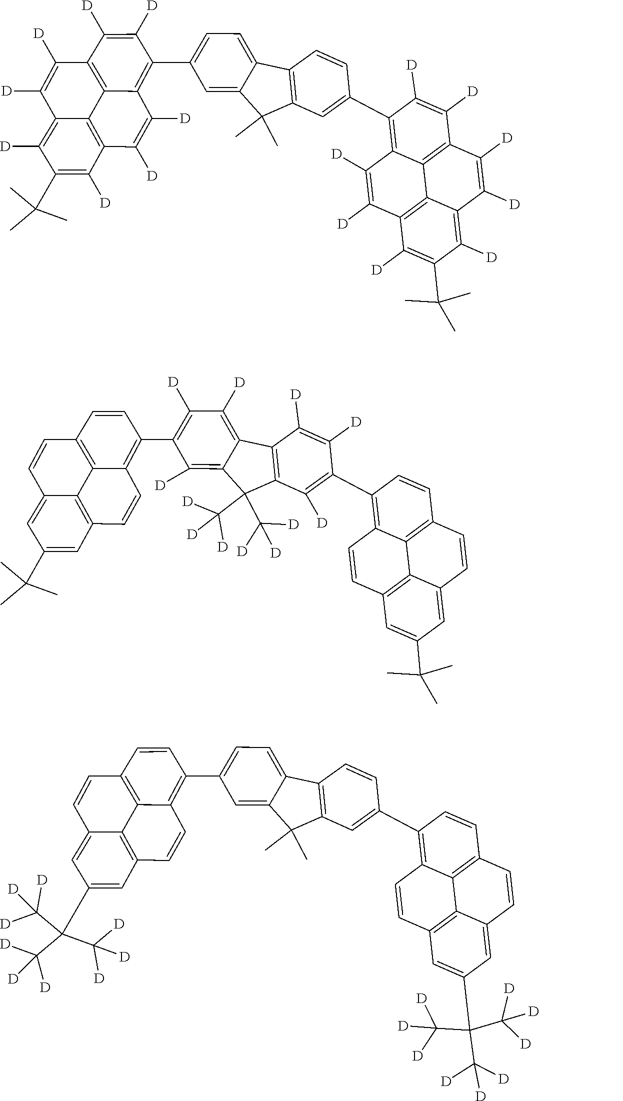

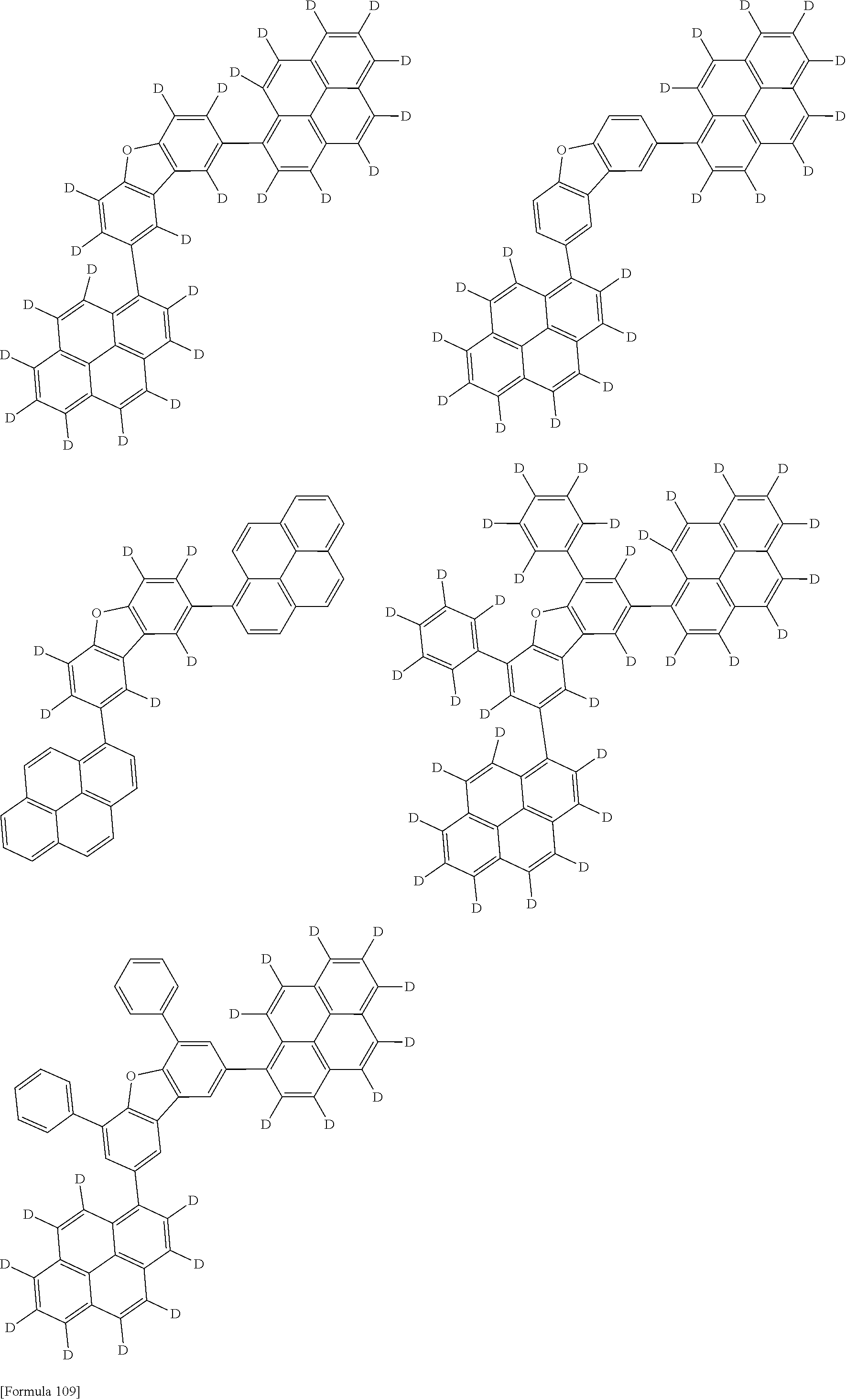

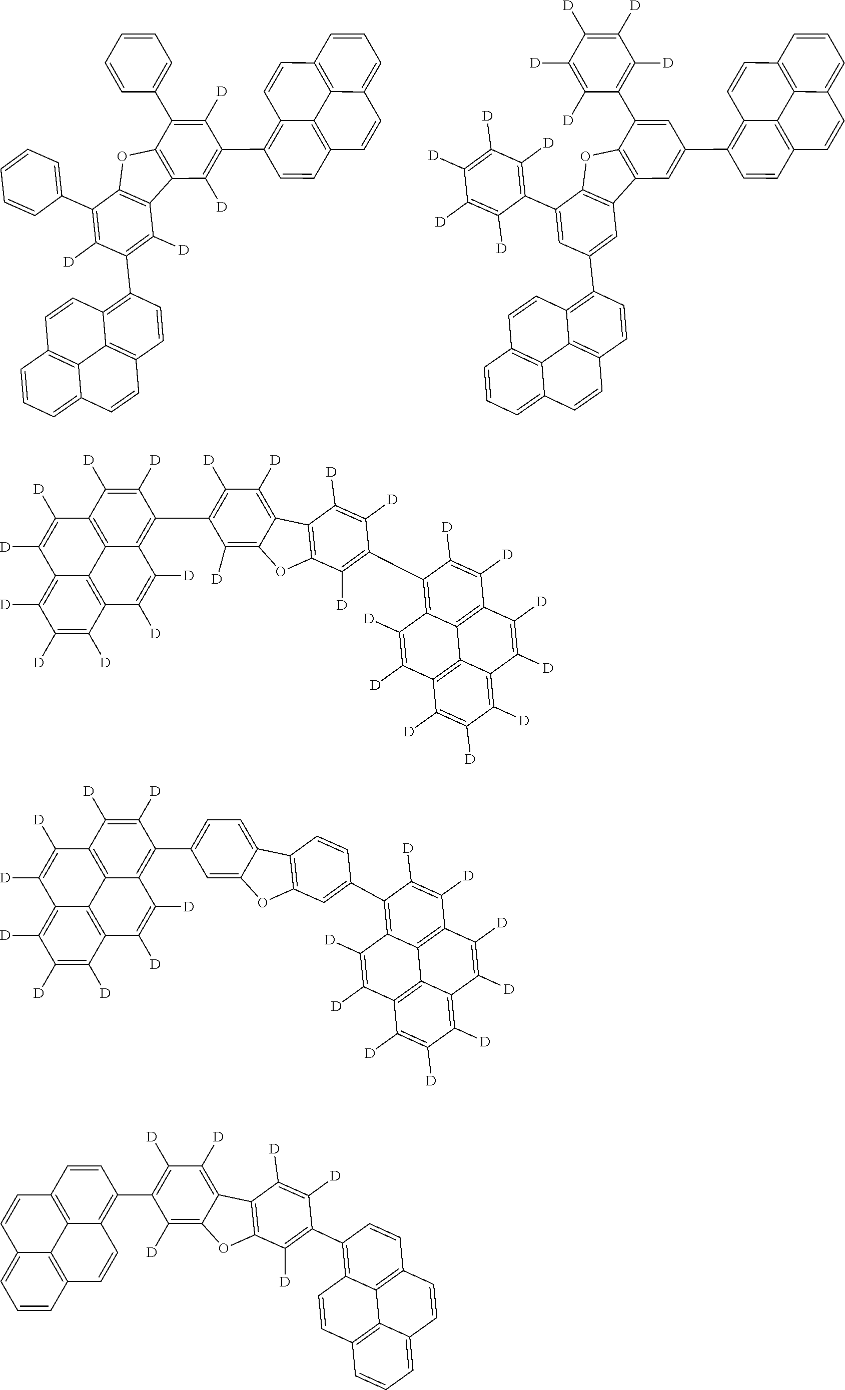

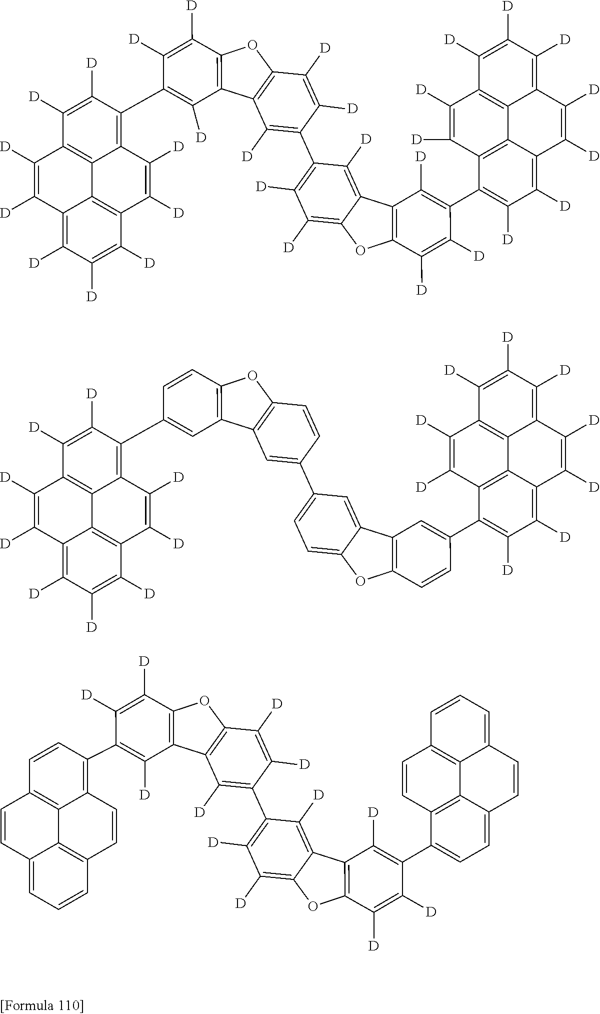

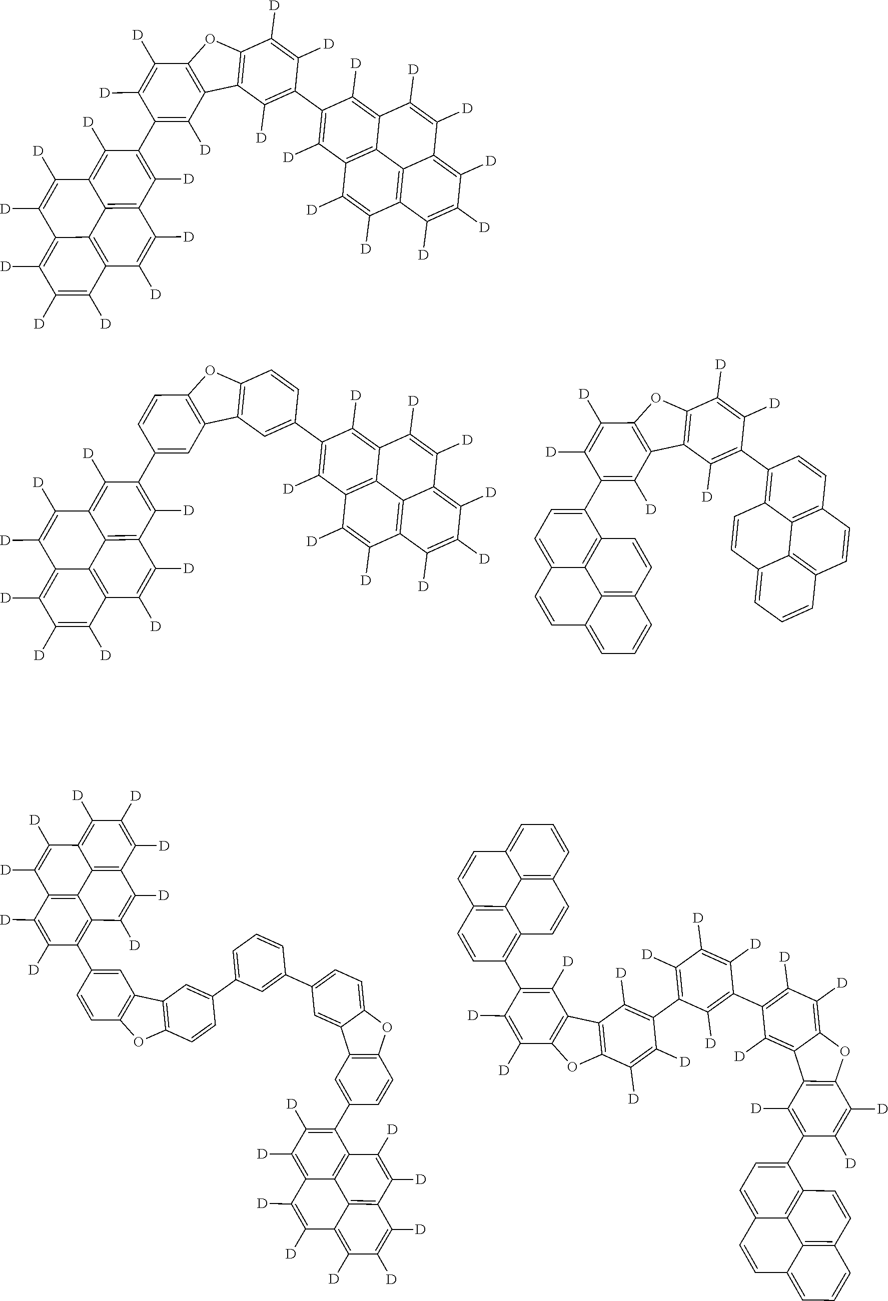

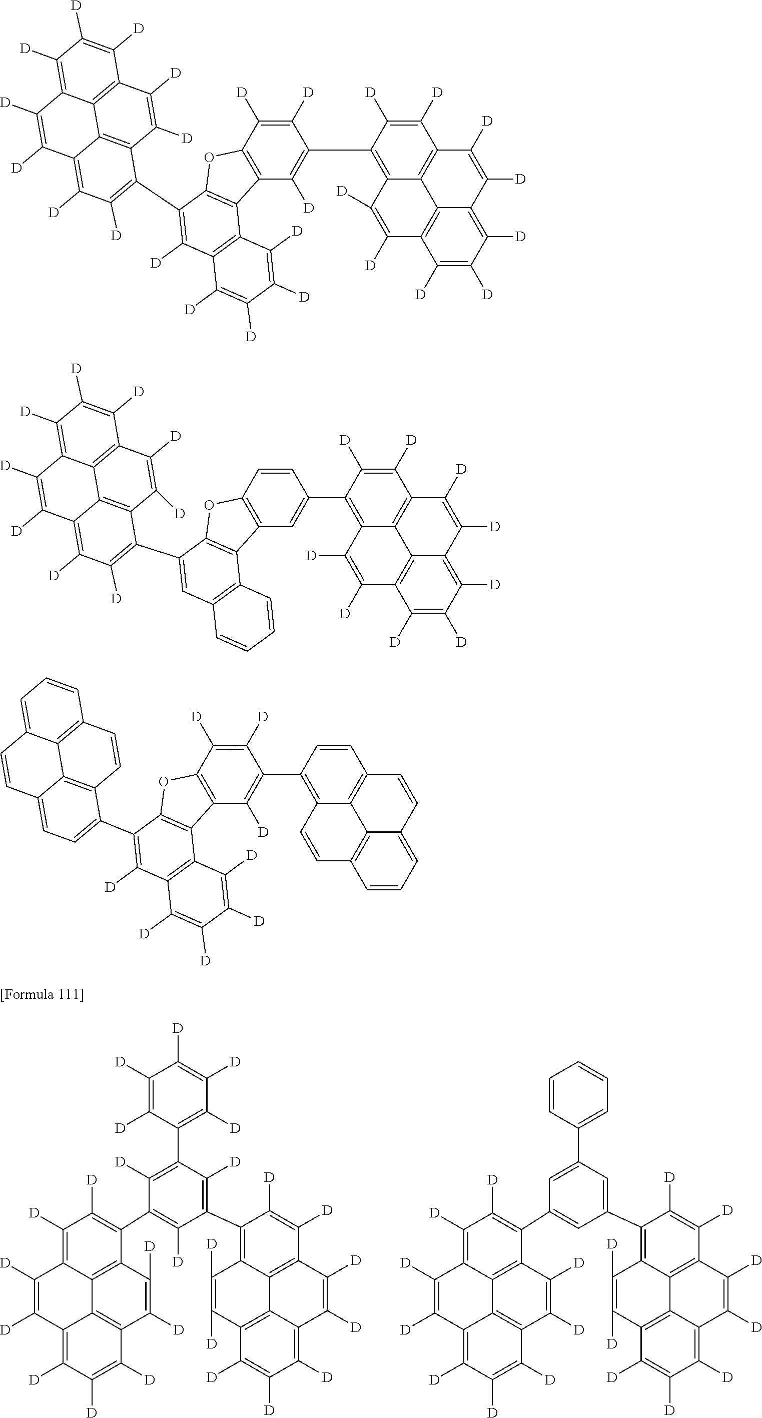

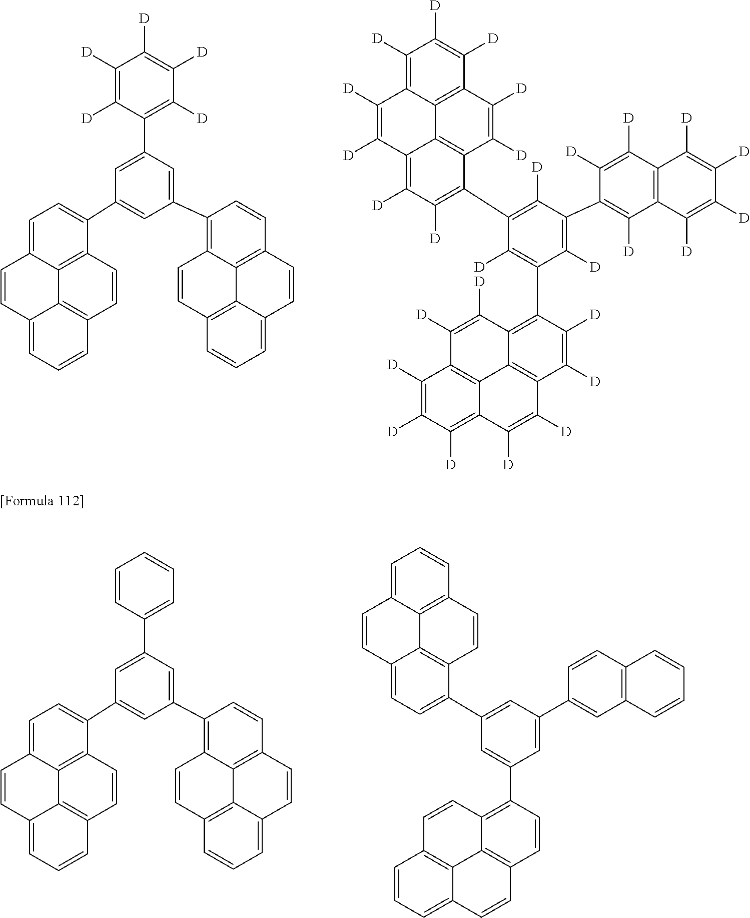

### Compound Represented by Formula (1X)

In an exemplary embodiment, the compound having the fused ring that includes four or more rings is a compound represented by a formula (1X) below and having at least one group represented by a formula (11X) below.

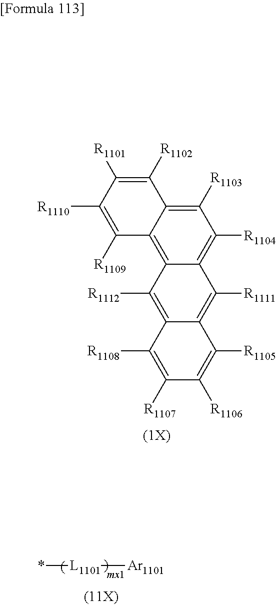

In the formula (1X):
R₁₁₀₁ to R₁₁₁₂ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11X);
at least one of R₁₁₀₁ to R₁₁₁₂ is a group represented by the formula (11X);
when a plurality of groups represented by the formula (11X) are present, the plurality of groups represented by the formula (11X) are mutually the same or different;
L₁₁₀₁ is a single bond, a substituted or unsubstituted arylene group having 6 to 50 ring carbon atoms, or a substituted or unsubstituted divalent heterocyclic group having 5 to 50 ring atoms;
Ar₁₁₀₁ is a substituted or unsubstituted aryl group having 6 to 50 ring carbon atoms, or a substituted or unsubstituted heterocyclic group having 5 to 50 ring atoms;
mx1 is 1, 2, 3, 4, or 5;
when two or more L₁₁₀₁ are present, the two or more L₁₁₀₁ are mutually the same or different;
when two or more Ar₁₁₀₁ are present, the two or more Ar₁₁₀₁ are mutually the same or different; and
* in the formula (11X) represents a bonding position to a benz[a]anthracene ring in the formula (1X).

In the compound represented by the formula (1X), R₉₀₁, R₉₀₂, R₉₀₃, R₉₀₄, R₉₀₅, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In the compound represented by the formula (1X), at least one of R₁₁₀₁ to R₁₁₁₂ not being the group represented by the formula (11X) is also preferably a deuterium atom.

In the compound represented by the formula (1X), L₁₁₀₁ also preferably has at least one deuterium atom.

In the compound represented by the formula (1X), Ar₁₁₀₁ also preferably has at least one deuterium atom.

In the compound represented by the formula (1X), Ar₁₁₀₁ is preferably a substituted or unsubstituted aryl group having 6 to 50 ring carbon atoms.

In the compound represented by the formula (1X), Ar₁₁₀₁ is preferably a substituted or unsubstituted phenyl group, a substituted or unsubstituted naphthyl group, a substituted or unsubstituted biphenyl group, a substituted or unsubstituted terphenyl group, a substituted or unsubstituted benz[a]anthryl group, a substituted or unsubstituted pyrenyl group, a substituted or unsubstituted phenanthryl group, or a substituted or unsubstituted fluorenyl group.

The compound represented by the formula (1X) is also preferably represented by a formula (101X) below.

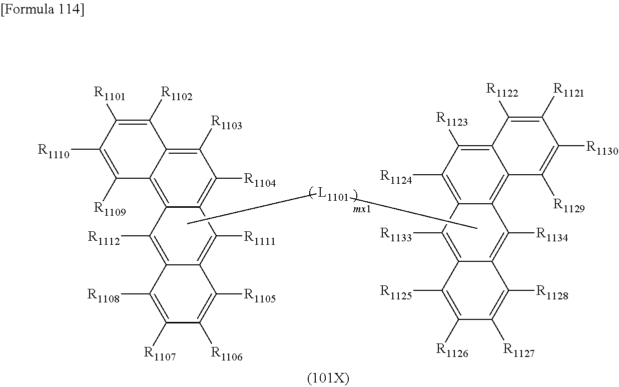

In the formula (101X):
one of R₁₁₁₁ and R₁₁₁₂ represents a bonding position to L₁₁₀₁ and one of R₁₁₃₃ and R₁₁₃₄ represents a bonding position to L₁₁₀₁;
R₁₁₀₁ to R₁₁₁₀, R₁₁₂₁ to R₁₁₃₀, R₁₁₁₁ or R₁₁₁₂ not being the bonding position to L₁₁₀₁, and R₁₁₃₃ or R₁₁₃₄ not being the bonding position to L₁₁₀₁ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₁₁₀₁ is a single bond, a substituted or unsubstituted arylene group having 6 to 50 ring carbon atoms, or a substituted or unsubstituted divalent heterocyclic group having 5 to 50 ring atoms;
mx1 is 1, 2, 3, 4, or 5; and
when two or more L₁₁₀₁ are present, the two or more L₁₁₀₁ are mutually the same or different.

In the compound represented by the formula (1X), L₁₁₀₁ is preferably a single bond, or a substituted or unsubstituted arylene group having 6 to 50 ring carbon atoms.

In the compound represented by the formula (1X), the group represented by the formula (11X) is also preferably a group represented by a formula (11AX) below or a group represented by a formula (11 BX) below.

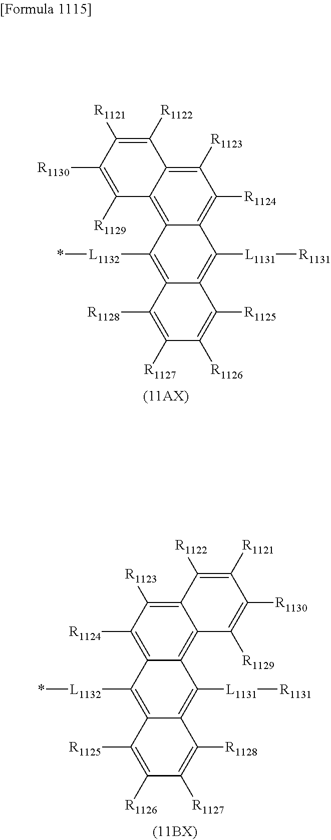

In the formulae (11AX) and (11BX):
R₁₁₂₁ to R₁₁₃₁ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
when a plurality of groups represented by the formula (11 AX) are present, the plurality of groups represented by the formula (11AX) are mutually the same or different;
when a plurality of groups represented by the formula (11 BX) are present, the plurality of groups represented by the formula (11 BX) are mutually the same or different;
L₁₁₃₁ and L₁₁₃₂ are each independently a single bond, a substituted or unsubstituted arylene group having 6 to 50 ring carbon atoms, or a substituted or unsubstituted divalent heterocyclic group having 5 to 50 ring atoms; and
* in each of the formulae (11 AX) and (11 BX) represents a bonding position to a benz[a]anthracene ring in the formula (1X).

The compound represented by the formula (1X) is also preferably represented by a formula (103X) below.

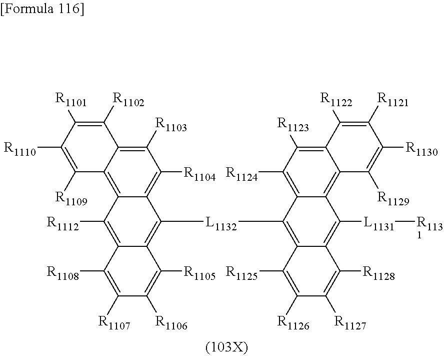

In the formula (103X):
R₁₁₀₁ to R₁₁₁₀ and R₁₁₁₂ respectively represent the same as R₁₁₀₁ to R₁₁₁₀ and R₁₁₁₂ in the formula (1X); and
R₁₁₂₁ to R₁₁₃₁, L₁₁₃₁, and L₁₁₃₂ respectively represent the same as R₁₁₂₁ to R₁₁₃₁, L₁₁₃₁, and L₁₁₃₂ in the formula (11 BX).

In the compound represented by the formula (1X), L₁₁₃₁ is also preferably a substituted or unsubstituted arylene group having 6 to 50 ring carbon atoms.

In the compound represented by the formula (1X), L₁₁₃₂ is also preferably a substituted or unsubstituted arylene group having 6 to 50 ring carbon atoms.

In the compound represented by the formula (1X), it is also preferable that two or more of R₁₁₀₁ to R₁₁₁₂ are each a group represented by the formula (11X).

In the compound represented by the formula (1X), it is preferable that two or more of R₁₁₀₁ to R₁₁₁₂ are each a group represented by the formula (11X); and Ar₁₁₀₁ in the formula (11X) is a substituted or unsubstituted aryl group having 6 to 50 ring carbon atoms.

In the compound represented by the formula (1X), it is also preferable that R₁₁₁₁ and R₁₁₁₂ are each a group represented by the formula (11X).

The compound represented by the formula (1X) is also preferably represented by a formula (1101X) below.

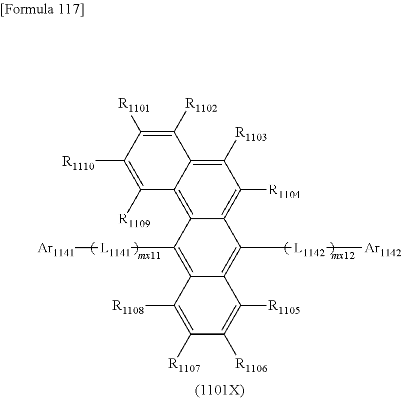

In the formula (1101X), R₁₁₀₁ to R₁₁₁₀ each independently represent the same as R₁₁₀₁ to R₁₁₁₀ in the formula (1X); Ar₁₁₄₁ and Ar₁₁₄₂ each independently represent the same as Ar₁₁₀₁ in the formula (11X); L₁₁₄₁ and L₁₁₄₂ each independently represent the same as L₁₁₀₁ in the formula (11X); and mx11 and mx12 each independently represent the same as mx1 in the formula (11X).

In the compound represented by the formula (1X), it is also preferable that Ar₁₁₀₁ is not a substituted or unsubstituted benz[a]anthryl group; L₁₁₀₁ is not a substituted or unsubstituted benz[a]anthrylene group; and the substituted or unsubstituted aryl group having 6 to 50 ring carbon atoms as R₁₁₀₁ to R₁₁₁₀ not being the group represented by the formula (11X) is not a substituted or unsubstituted benz[a]anthryl group.

In the compound represented by the formula (1X), R₁₁₀₁ to R₁₁₁₂ not being the group represented by the formula (11X) are preferably each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the compound represented by the formula (1X), R₁₁₀₁ to R₁₁₁₂ not being the group represented by the formula (11X) are each preferably a hydrogen atom, a substituted or unsubstituted alkyl group having 1 to 50 carbon atoms, or a substituted or unsubstituted cycloalkyl group having 3 to 50 ring carbon atoms.

In the compound represented by the formula (1X), R₁₁₀₁ to R₁₁₁₂ not being the group represented by the formula (11X) are each preferably a hydrogen atom.

### Method of Manufacturing Compound Represented by Formula (1X)

The compound represented by the formula (1X) can be manufactured by a known method. The compound represented by the formula (1X) can also be manufactured based on a known method through a known alternative reaction using a known material(s) tailored for the target compound.

### Specific Examples of Compound Represented by Formula (1X)

Specific examples of the compound represented by the formula (1X) include the following compounds. It should however be noted that the invention is not limited to the specific examples.

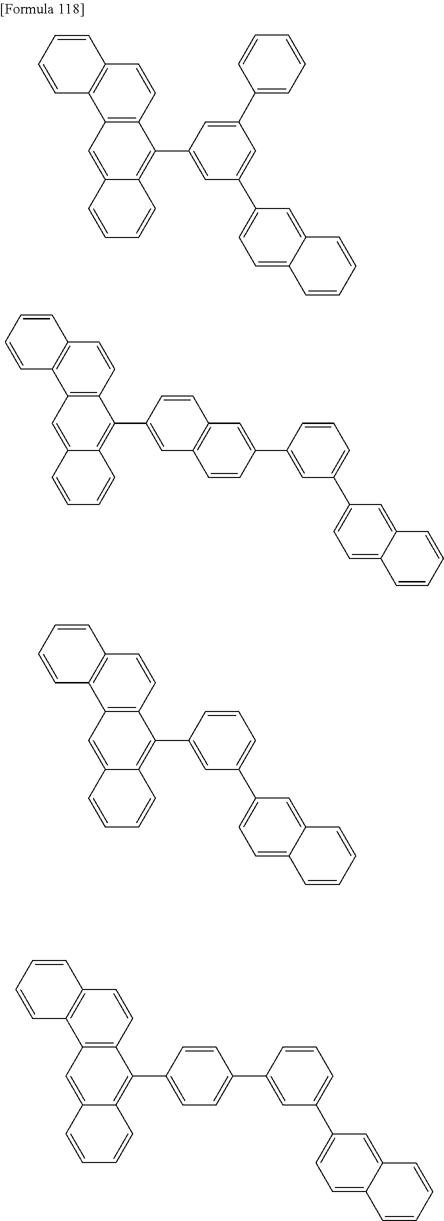

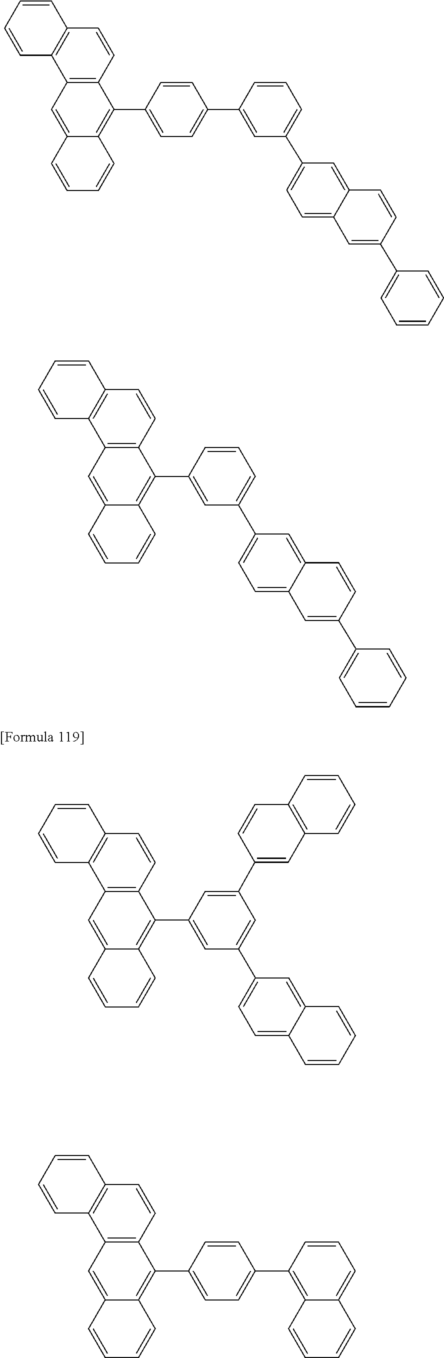

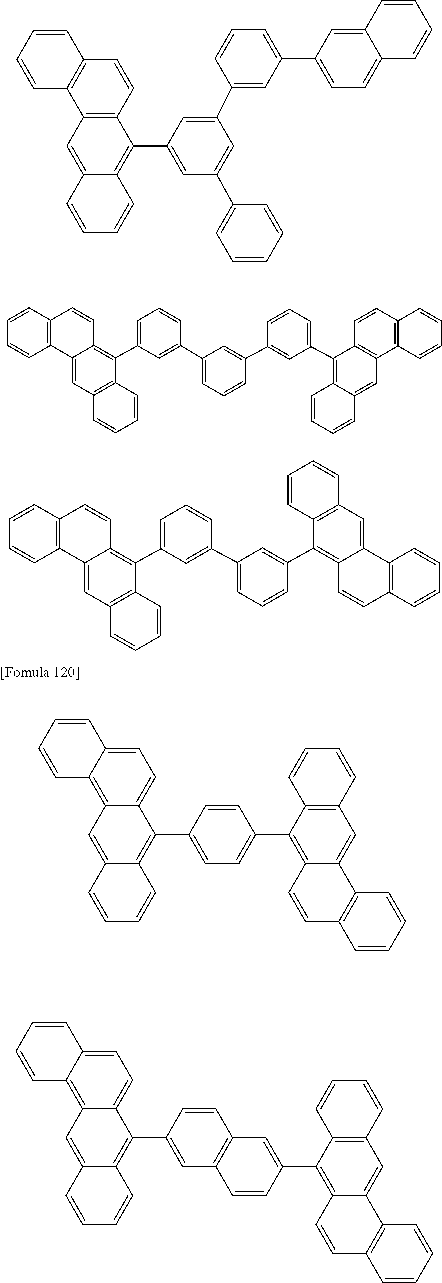

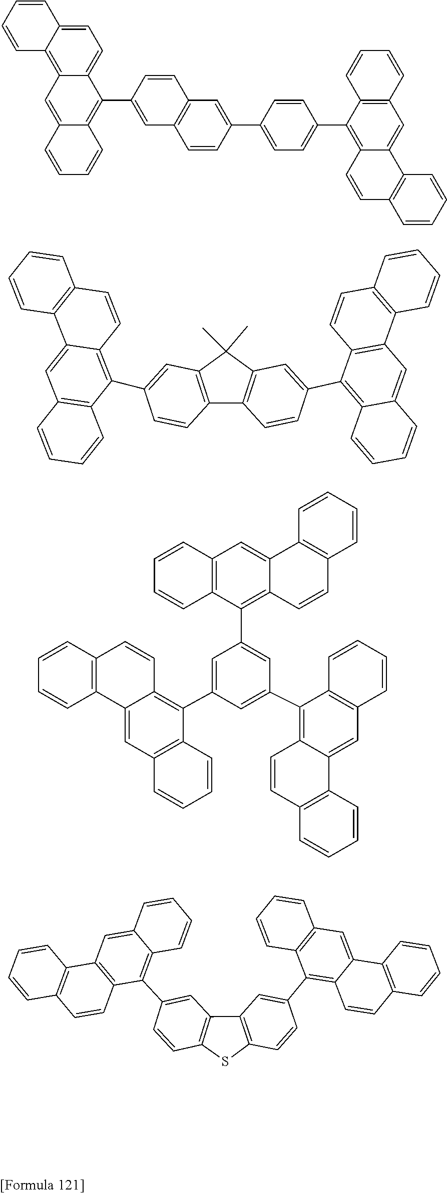

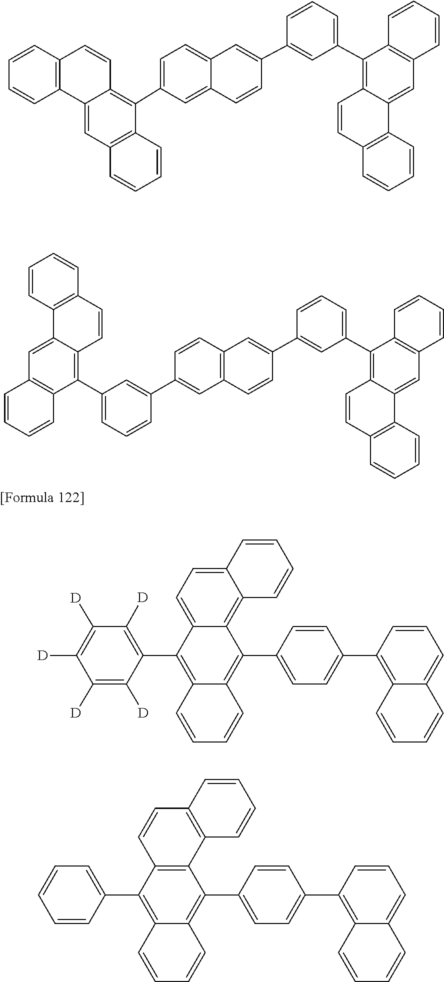

### Compound Represented by Formula (14X)

In an exemplary embodiment, the compound having the fused ring that includes four or more rings is a compound represented by a formula (14X) below and having at least one group represented by a formula (141) below.

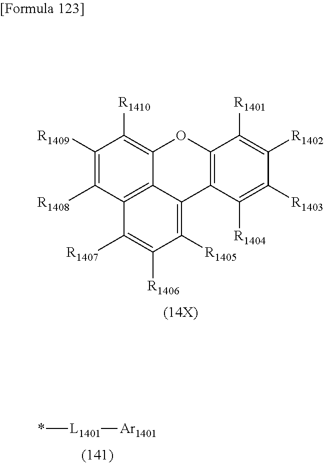

In the formula (14X):
R₁₄₀₁ to R₁₄₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (141);
at least one of R₁₄₀₁ to R₁₄₁₀ is a group represented by the formula (141);
when a plurality of groups represented by the formula (141) are present, the plurality of groups represented by the formula (141) are mutually the same or different;
L₁₄₀₁ is a single bond, a substituted or unsubstituted arylene group having 6 to 50 ring carbon atoms, or a substituted or unsubstituted divalent heterocyclic group having 5 to 50 ring atoms;
Ar₁₄₀₁ is a substituted or unsubstituted aryl group having 6 to 50 ring carbon atoms, or a substituted or unsubstituted heterocyclic group having 5 to 50 ring atoms;
mx4 is 0, 1, 2, 3, 4 or 5;
when two or more L₁₄₀₁ are present, the two or more L₁₄₀₁ are mutually the same or different;
when two or more Ar₁₄₀₁ are present, the two or more Ar₁₄₀₁ are mutually the same or different; and
* in the formula (141) represents a bonding position to a ring represented by the formula (14X).

In the compound represented by the formula (14X), R₉₀₁, R₉₀₂, R₉₀₃, R₉₀₄, R₉₀₅, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In the compound represented by the formula (14X), at least one of R₁₄₀₁ to R₁₄₁₀ not being the group represented by the formula (141) is also preferably a deuterium atom.

In the compound represented by the formula (14X), L₁₄₀₁ also preferably has at least one deuterium atom.

In the compound represented by the formula (14X), Ar₁₄₀₁ also preferably has at least one deuterium atom.

In the compound represented by the formula (14X), Ar₁₄₀₁ is preferably a substituted or unsubstituted aryl group having 6 to 50 ring carbon atoms.

In the compound represented by the formula (14X), it is also preferable that two or more of R₁₄₀₁ to R₁₄₁₀ are each a group represented by the formula (141).

In the compound represented by the formula (14X), it is preferable that two or more of R₁₄₀₁ to R₁₄₁₀ are each a group represented by the formula (141); and Ar₁₄₀₁ in the formula (141) is a substituted or unsubstituted aryl group having 6 to 50 ring carbon atoms.

In the compound represented by the formula (14X), L₁₄₀₁ is preferably a single bond, or a substituted or unsubstituted arylene group having 6 to 50 ring carbon atoms.

In the compound represented by the formula (14X), R₁₄₀₁ to R₁₄₁₀ not being the group represented by the formula (141) are preferably each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the compound represented by the formula (14X), R₁₄₀₁ to R₁₄₁₀ not being the group represented by the formula (141) are each preferably a hydrogen atom.

### Method of Manufacturing Compound Represented by Formula (14X)

The compound represented by the formula (14X) can be manufactured by a known method. The compound represented by the formula (14X) can also be manufactured based on a known method through a known alternative reaction using a known material(s) tailored for the target compound.

### Specific Examples of Compound Represented by Formula (14X)

Specific examples of the compound represented by the formula (14X) include the following compounds. It should however be noted that the invention is not limited to the specific examples.

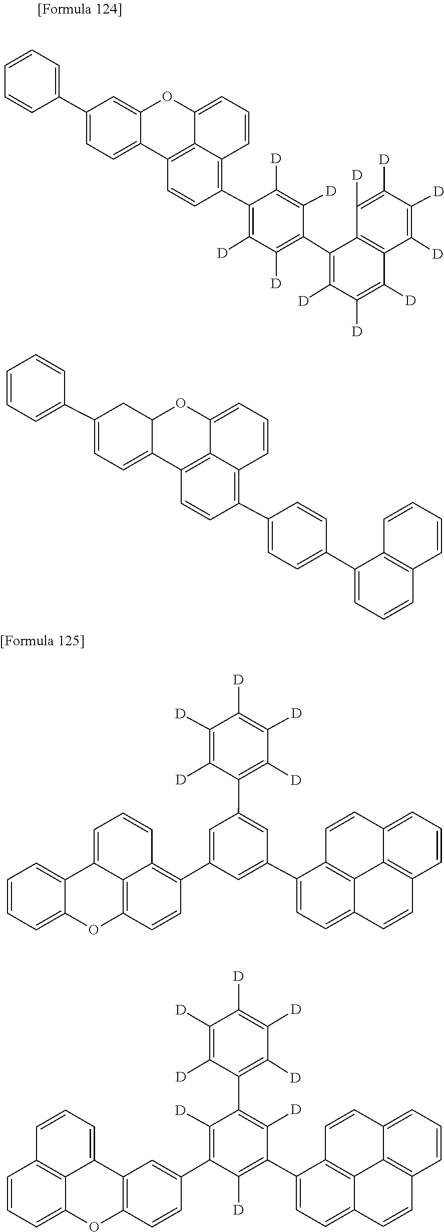 [SMILES: [2H]c1c([2H])c(-c2c([2H])c([2H])c([2H])c3c([2H])c([2H])c([2H])c([2H])c23)c([2H])c([2H])c1-c1ccc2c3c(cccc13)Oc1cc(-c3ccccc3)ccc1-2 ; C1=C(c2ccccc2)CC2Oc3cccc4c(-c5ccc(-c6cccc7ccccc67)cc5)ccc(c34)C2=C1 ; [2H]c1c([2H])c([2H])c(-c2cc(-c3ccc4c5c(cccc35)-c3ccccc3O4)cc(-c3ccc4ccc5cccc6ccc3c4c56)c2)c([2H])c1[2H] ; [2H]c1c([2H])c([2H])c(-c2c([2H])c(-c3ccc4c(c3)-c3cccc5cccc(c35)O4)c([2H])c(-c3ccc4ccc5cccc6ccc3c4c56)c2[2H])c([2H])c1[2H]]

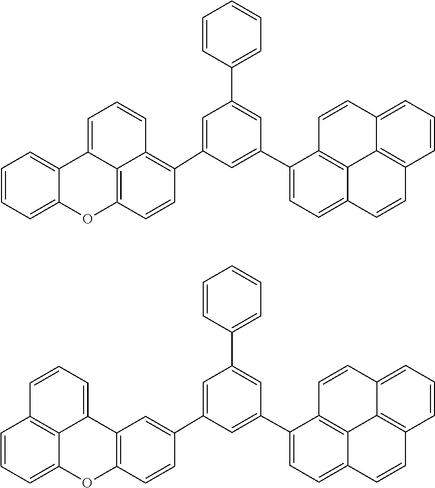 [SMILES: c1ccc(-c2cc(-c3ccc4c5c(cccc35)-c3ccccc3O4)cc(-c3ccc4ccc5cccc6ccc3c4c56)c2)cc1 ; c1ccc(-c2cc(-c3ccc4c(c3)-c3cccc5cccc(c35)O4)cc(-c3ccc4ccc5cccc6ccc3c4c56)c2)cc1]

### Compound Represented by Formula (2)

In the organic electroluminescence device according to the exemplary embodiment, the second emitting layer preferably contains a compound represented by a formula (2) below.

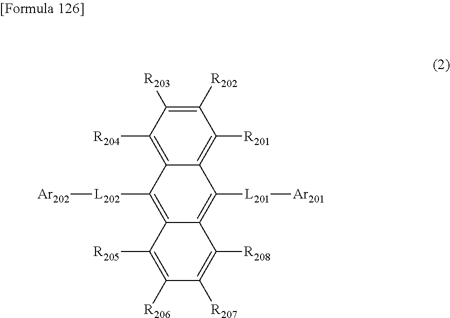

In the formula (2):
R₂₀₁ to R₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms; and
Ar₂₀₁ and Ar₂₀₂ are each independently a substituted or unsubstituted aryl group having 6 to 50 ring carbon atoms, or a substituted or unsubstituted heterocyclic group having 5 to 50 ring atoms.

In the compound represented by the formula (2), R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₅ are present, the plurality of R₉₀₅ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In the organic EL device according to the exemplary embodiment, it is preferable that:
R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, or a nitro group; L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms; and
Ar₂₀₁ and Ar₂₀₂ are each independently a substituted or unsubstituted aryl group having 6 to 50 ring carbon atoms, or a substituted or unsubstituted heterocyclic group having 5 to 50 ring atoms.

In the organic EL device according to the exemplary embodiment, it is preferable that:
L₂₀₁ and L₂₀₂ are each independently a single bond, or a substituted or unsubstituted arylene group having 6 to 50 ring carbon atoms; and
Ar₂₀₁ and Ar₂₀₂ are each independently a substituted or unsubstituted aryl group having 6 to 50 ring carbon atoms.

In the organic EL device according to the exemplary embodiment, it is preferable that Ar₂₀₁ and Ar₂₀₂ are each independently a phenyl group, naphthyl group, phenanthryl group, biphenyl group, terphenyl group, diphenylfluorenyl group, dimethylfluorenyl group, benzodiphenylfluorenyl group, benzodimethylfluorenyl group, dibenzofuranyl group, dibenzothienyl group, naphthobenzofuranyl group, or naphthobenzothienyl group.

In the organic EL device according to the exemplary embodiment, at least one group of L₂₀₁, L₂₀₂, Ar₂₀₁, or Ar₂₀₂ preferably has at least one deuterium atom.

In the organic EL device according to the exemplary embodiment, at least one of Ar₂₀₁ or Ar₂₀₂ is also preferably a group represented by a formula (21), a formula (22), a formula (23), or a formula (24) below.

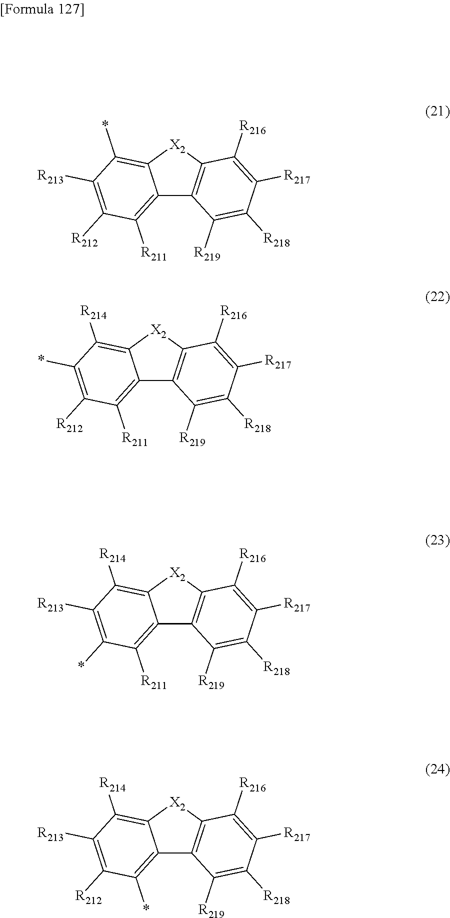

In the formulae (21) to (24),
X₂ is an oxygen atom, a sulfur atom, CR₂₃₁R₂₃₂, or NR₂₃₃;
at least one combination of adjacent two or more of R₂₁₁ to R₂₁₄ and R₂₁₆ to R₂₁₉ are mutually bonded to form a substituted or unsubstituted monocyclic ring, mutually bonded to form a substituted or unsubstituted fused ring, or not mutually bonded;
a combination of R₂₃₁ and R₂₃₂ are mutually bonded to form a substituted or unsubstituted monocyclic ring, mutually bonded to form a substituted or unsubstituted fused ring, or not mutually bonded;
R₂₁₁ to R₂₁₄ and R₂₁₆ to R₂₁₉ forming neither the substituted or unsubstituted monocyclic ring nor the substituted or unsubstituted fused ring; R₂₃₁ and R₂₃₂ forming neither the substituted or unsubstituted monocyclic ring nor the substituted or unsubstituted fused ring; and R₂₃₃ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
* in each of the formulae (21) to (24) represents a bonding position to L₂₀₁ or L₂₀₂.

At least one of R₂₁₁ to R₂₁₄ or R₂₁₆ to R₂₁₉ is preferably a substituted or unsubstituted aryl group having 6 to 50 ring carbon atoms that has at least one deuterium atom, more preferably a substituted or unsubstituted phenyl group having at least one deuterium atom, and further preferably a phenyl group having four deuterium atoms.

In the organic EL device according to the exemplary embodiment, at least one of L₂₀₁ or L₂₀₂ is also preferably a group represented by a formula (L21), a formula (L22), a formula (L23), or a formula (L24) below.

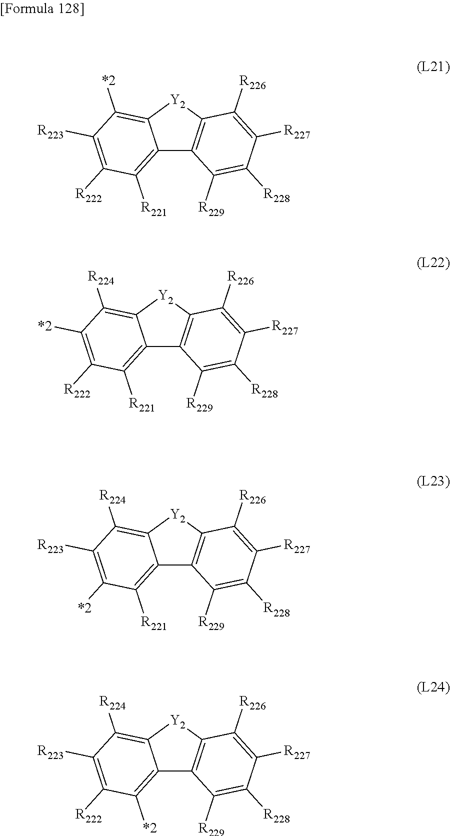

In the formulae (L21) to (L24),
Y₂ is an oxygen atom, a sulfur atom, CR₂₄₁R₂₄₂, or NR₂₄₃;
at least one of R₂₂₁ to R₂₂₄ or R₂₂₆ to R₂₂₉ is Ar₂₀₁ or Ar₂₀₂;
at least one combination of adjacent two or more of R₂₂₁ to R₂₂₄ and R₂₂₆ to R₂₂₉ being neither Ar₂₀₁ nor Ar₂₀₂ are mutually bonded to form a substituted or unsubstituted monocyclic ring, mutually bonded to form a substituted or unsubstituted fused ring, or not mutually bonded;
a combination of R₂₄₁ and R₂₄₂ are mutually bonded to form a substituted or unsubstituted monocyclic ring, mutually bonded to form a substituted or unsubstituted fused ring, or not mutually bonded;
R₂₂₁ to R₂₂₄ and R₂₂₆ to R₂₂₉ being neither Ar₂₀₁ nor Ar₂₀₂ and forming neither the substituted or unsubstituted monocyclic ring nor the substituted or unsubstituted fused ring; R₂₄₁ and R₂₄₂ forming neither the substituted or unsubstituted monocyclic ring nor the substituted or unsubstituted fused ring; and R₂₄₃ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
*2 in each of the formulae (L₂₁) to (L₂₄) represents a bonding position to an anthracene ring represented by the formula (2).

At least one of R₂₂₁ to R₂₂₄ or R₂₂₆ to R₂₂₉ is preferably a substituted or unsubstituted aryl group having 6 to 50 ring carbon atoms that has at least one deuterium atom, more preferably a substituted or unsubstituted phenyl group having at least one deuterium atom, and further preferably a phenyl group having four deuterium atoms.

In the organic EL device according to the exemplary embodiment, the compound represented by the formula (2) is preferably a compound represented by a formula (201), a formula (202), a formula (203), a formula (204), a formula (205), a formula (206), a formula (207), a formula (208), or a formula (209) below.

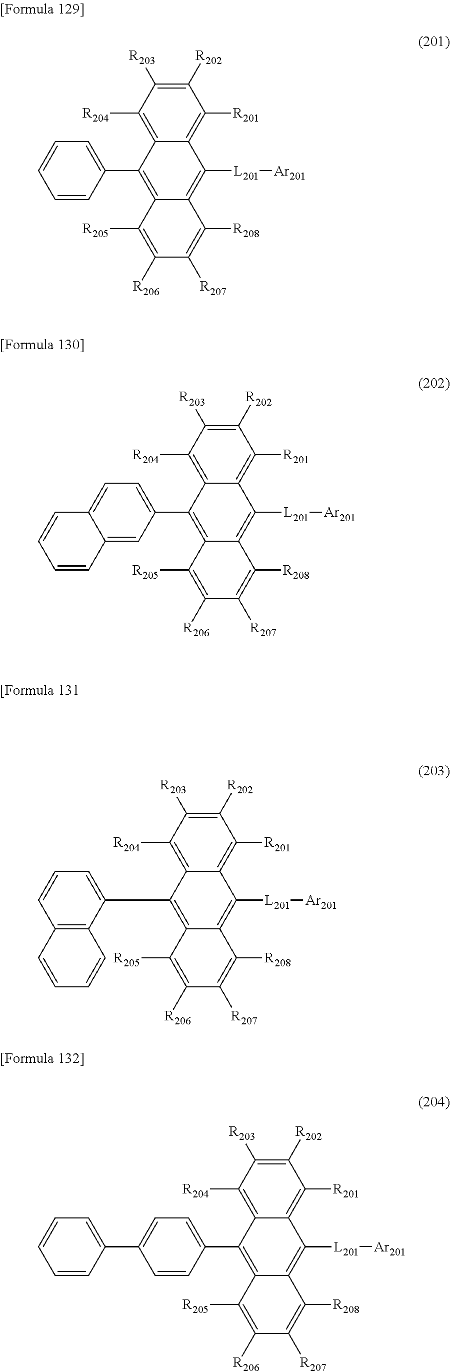

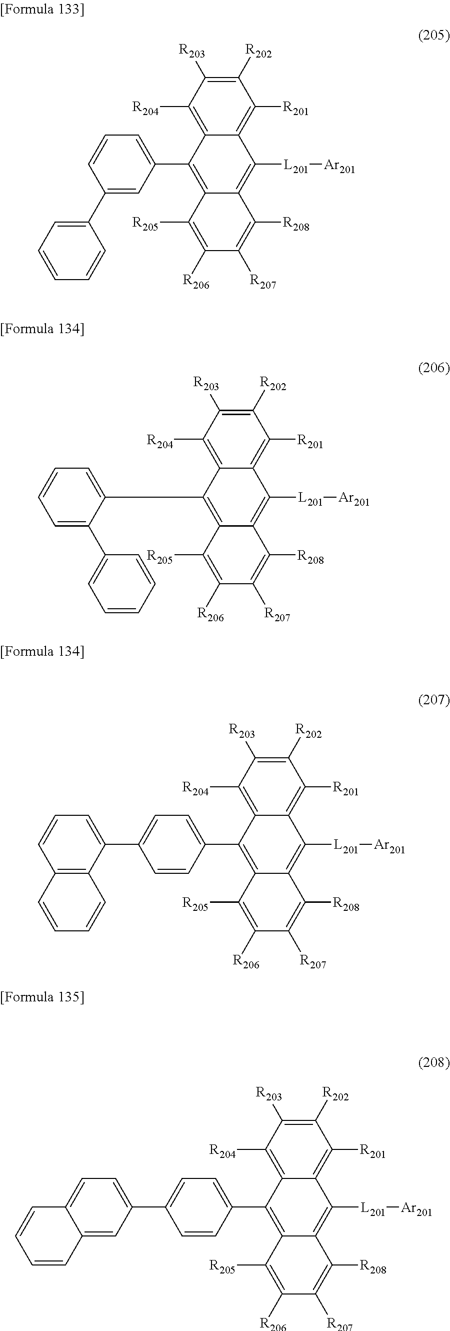

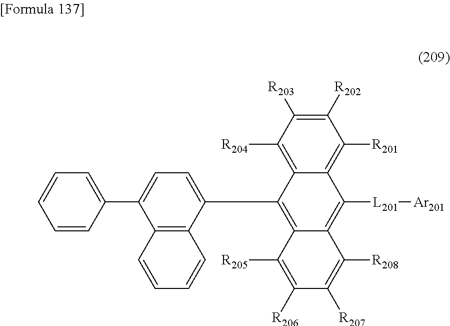

In the formulae (201) to (209):
L₂₀₁ and Ar₂₀₁ represent the same as L₂₀₁ and Ar₂₀₁ in the formula (2); and
R₂₀₁ to R₂₀₈ each independently represent the same as R₂₀₁ to R₂₀₈ in the formula (2).

The compound represented by the formula (2) is also preferably a compound represented by a formula (221), a formula (222), a formula (223), a formula (224), a formula (225), a formula (226), a formula (227), a formula (228), or a formula (229) below.

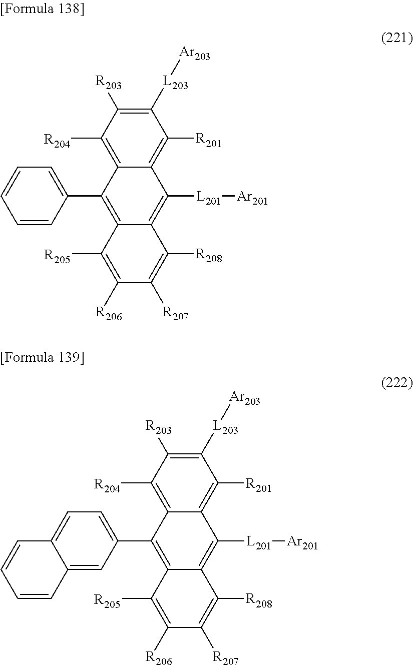

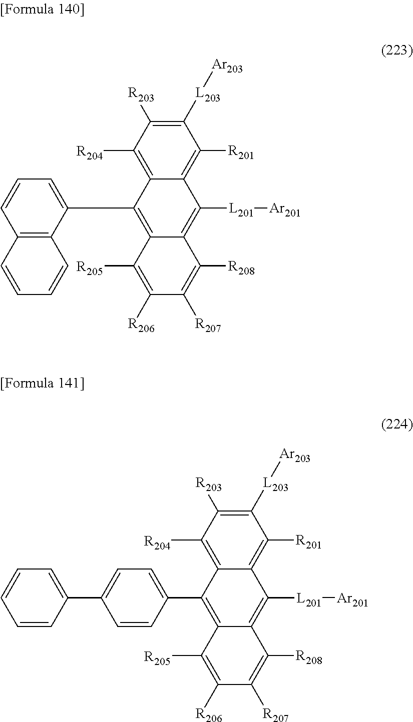

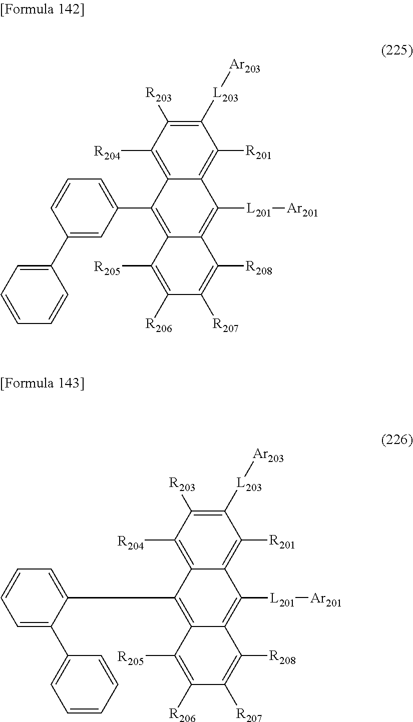

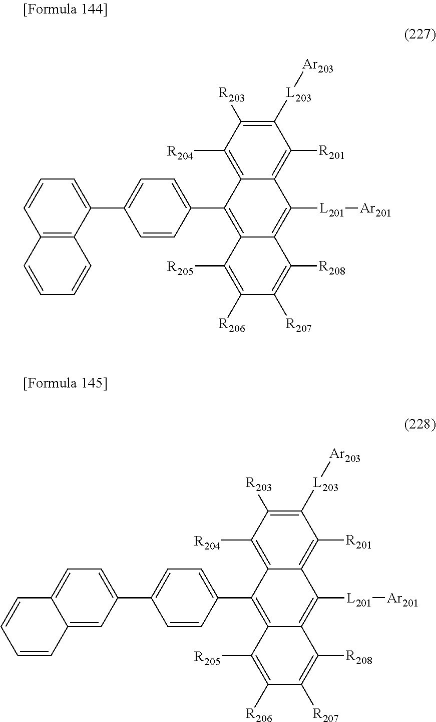

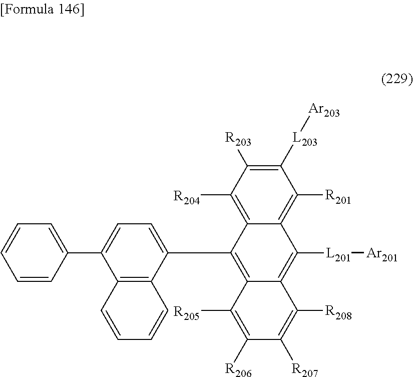

In the formulae (221), (222), (223), (224), (225), (226), (227), (228) and (229):
R₂₀₁ and R₂₀₃ to R₂₀₈ each independently represent the same as R₂₀₁ and R₂₀₃ to R₂₀ in the formula (2);
L₂₀₁ and Ar₂₀₁ respectively represent the same as L₂₀₁ and Ar₂₀₁ in the formula (2);
L₂₀₃ represents the same as L₂₀₁ in the formula (2);
L₂₀₃ and L₂₀₁ are mutually the same or different;
Ar₂₀₃ represents the same as Ar₂₀₁ in the formula (2); and
Ar₂₀₃ and Ar₂₀₁ are mutually the same or different.

The compound represented by the formula (2) is also preferably a compound represented by a formula (241), a formula (242), a formula (243), a formula (244), a formula (245), a formula (246), a formula (247), a formula (248), or a formula (249) below.

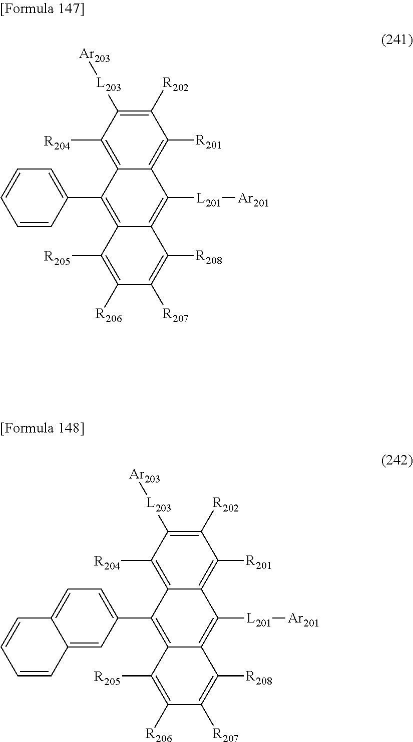

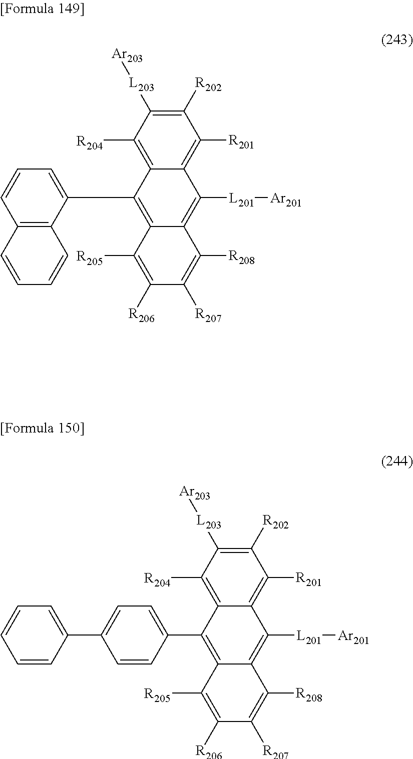

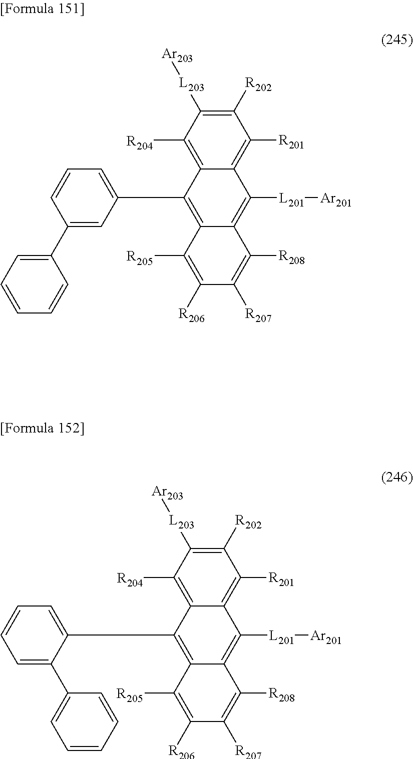

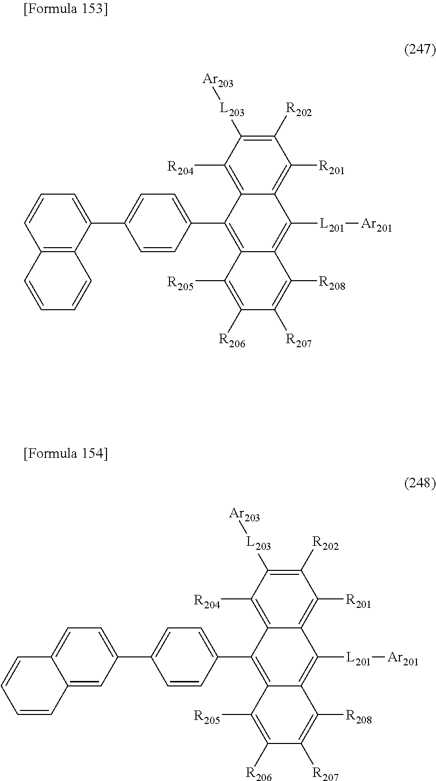

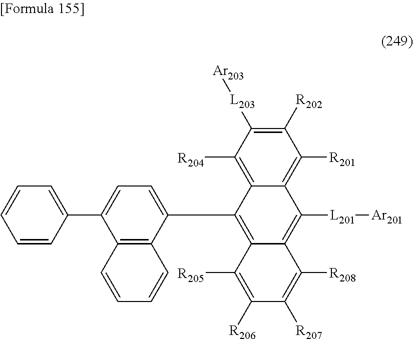

In the formulae (241), (242), (243), (244), (245), (246), (247), (248) and (249):
R₂₀₁, R₂₀₂ and R₂₀₄ to R₂₀₈ each independently represent the same as R₂₀₁, R₂₀₂ and R₂₀₄ to R₂₀₈ in the formula (2);
L₂₀₃ represents the same as L₂₀₁ in the formula (2);
L₂₀₁ and Ar₂₀₁ respectively represent the same as L₂₀₁ and Ar₂₀₁ in the formula (2);
L₂₀₃ and L₂₀₁ are mutually the same or different;
Ar₂₀₃ represents the same as Ar₂₀₁ in the formula (2); and
Ar₂₀₃ and Ar₂₀₁ are mutually the same or different.

In the compound represented by the formula (2), R₂₀₁ to R₂₀₈ not being the group represented by the formula (21) are preferably each independently a hydrogen atom, a substituted or unsubstituted alkyl group having 1 to 50 carbon atoms, a substituted or unsubstituted cycloalkyl group having 3 to 50 ring carbon atoms, or a group represented by —Si(R₉₀₁)(R₉₀₂)(R₉₀₃).

It is preferable that L₁₀₁ is a single bond, or an unsubstituted arylene group having 6 to 22 ring carbon atoms; and Ar₁₀₁ is a substituted or unsubstituted aryl group having 6 to 22 ring carbon atoms.

In the organic EL device according to the exemplary embodiment, R₂₀₁ to R₂₀₈ in the compound represented by the formula (2) are preferably each independently a hydrogen atom, a substituted or unsubstituted alkyl group having 1 to 50 carbon atoms, a substituted or unsubstituted cycloalkyl group having 3 to 50 ring carbon atoms, or a group represented by —Si(R₉₀₁)(R₉₀₂)(R₉₀₃).

In the organic EL device according to the exemplary embodiment, R₂₀₁ to R₂₀₈ in the compound represented by the formula (2) are each preferably a hydrogen atom.

In the compound represented by the formula (2), the groups specified to be “substituted or unsubstituted” are each preferably an “unsubstituted” group.

In the organic EL device according to the exemplary embodiment, for instance, Ar₂₀₁ in the compound represented by the formula (2) is a substituted or unsubstituted dibenzofuranyl group.

In the organic EL device according to the exemplary embodiment, for instance, Ar₂₀₁ in the second compound represented by the formula (2) is an unsubstituted dibenzofuranyl group.

In the organic EL device according to the exemplary embodiment, for instance, the compound represented by the formula (2) has at least one hydrogen atom that includes at least one deuterium atom.

In the organic EL device according to the exemplary embodiment, the compound represented by the formula (2) preferably has at least one deuterium atom.

In the organic EL device according to the exemplary embodiment, at least one of R₂₀₁ to R₂₀₈ is preferably a deuterium atom.

In the organic EL device according to the exemplary embodiment, at least one of L₂₀₁ or L₂₀₂ preferably has at least one deuterium atom.

In the organic electroluminescence device according to the exemplary embodiment, at least one of Ar₂₀₁ or Ar₂₀₂ preferably has at least one deuterium atom.

In the organic EL device according to the exemplary embodiment, for instance, L₂₀₁ in the compound represented by the formula (2) is one of TEMP-63 to TEMP-68

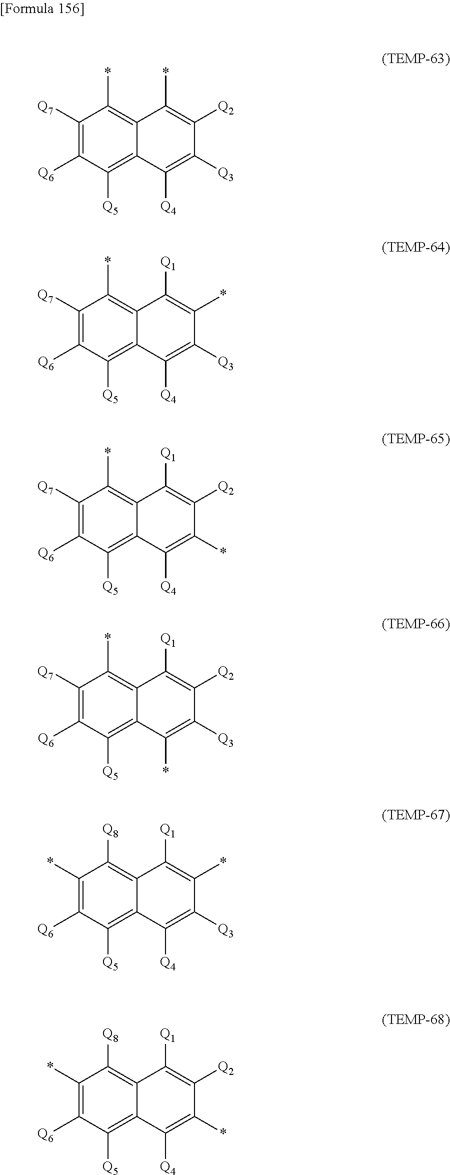

In the organic EL device according to the exemplary embodiment, for instance, Ar₂₀₁ in the compound represented by the formula (2) is at least one group selected from the group consisting of substituted or unsubstituted anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluoranthenyl group, benzofluoranthenyl group, and perylenyl group.

In the organic EL device according to the exemplary embodiment, for instance, Ar₂₀₁ in the compound represented by the formula (2) is a substituted or unsubstituted fluorenyl group.

In the organic EL device according to the exemplary embodiment, for instance, Ar₂₀₁ in the compound represented by the formula (2) is a substituted or unsubstituted xanthenyl group.

In the organic EL device according to the exemplary embodiment, for instance, Ar₂₀₁ in the compound represented by the formula (2) is a benzoxanthenyl group.

### Method of Manufacturing Compound Represented by Formula (2)

The compound represented by the formula (2) can be manufactured by a known method. The second compound can also be manufactured based on a known method through a known alternative reaction using a known material(s) tailored for the target compound.

### Specific Examples of Compound Represented by Formula (2)

Specific examples of the compound represented by the formula (2) include the following compounds. It should however be noted that the invention is not limited to the specific examples.

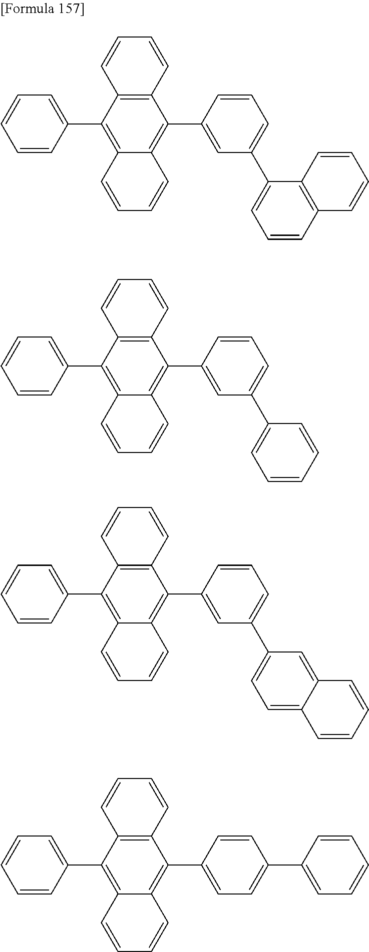

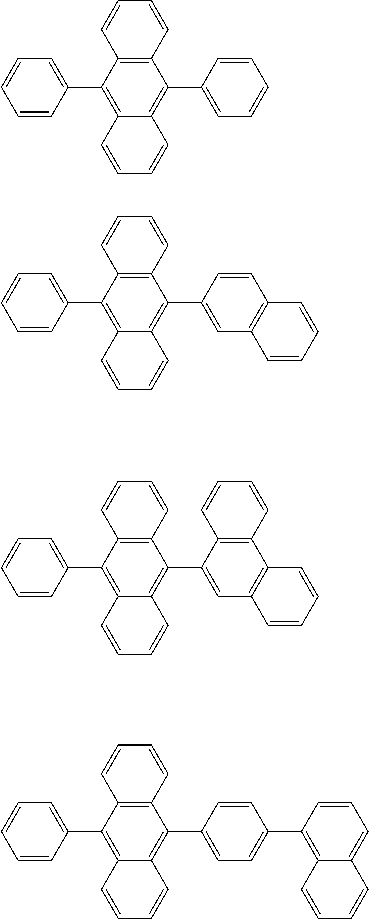

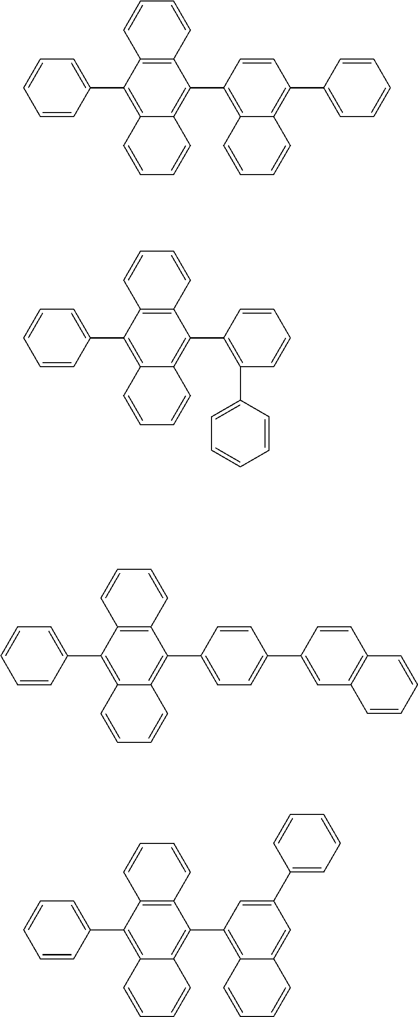

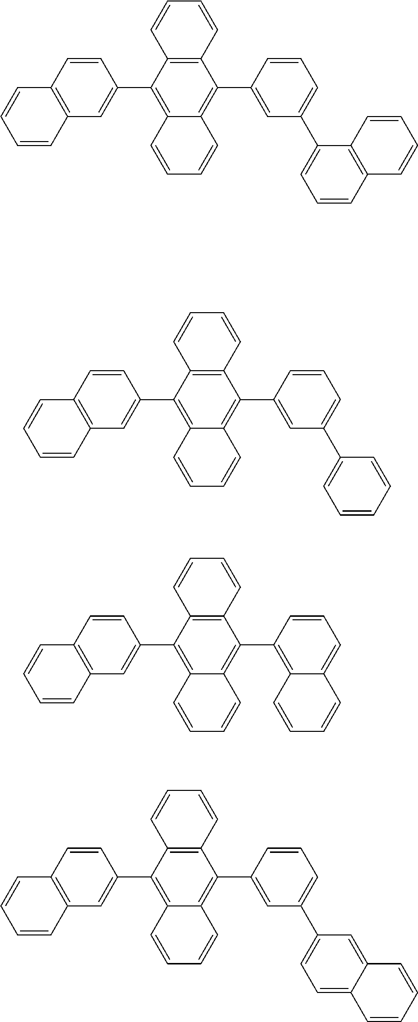

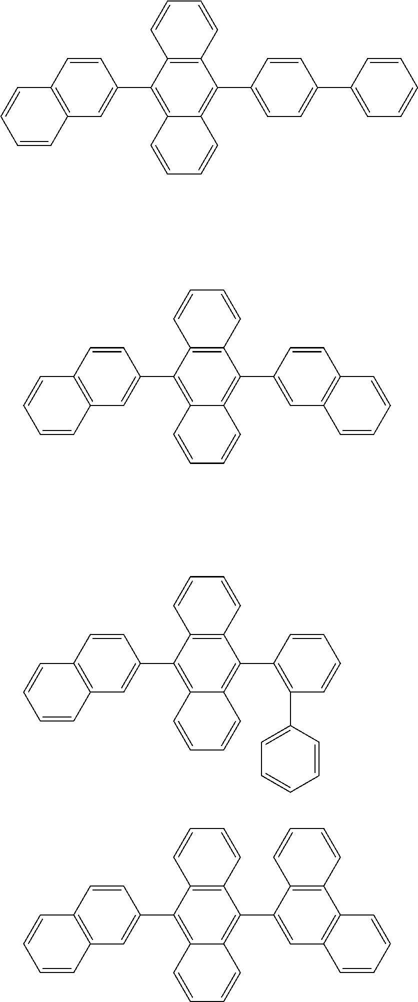

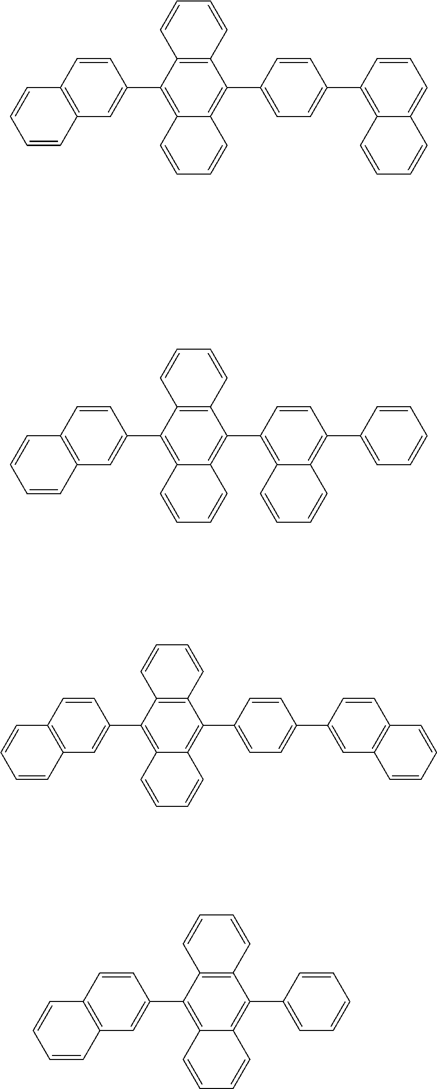

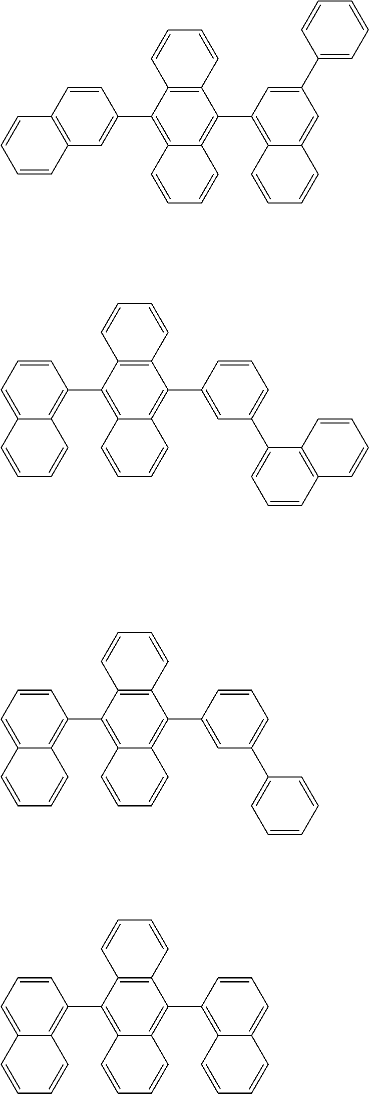

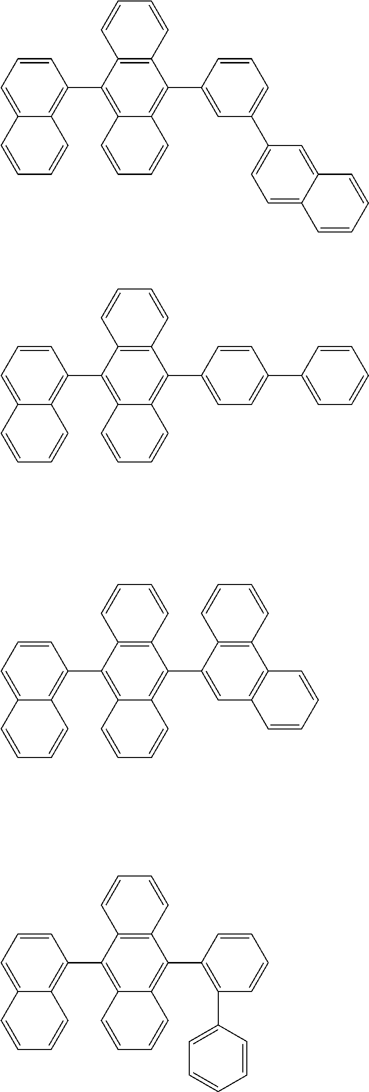

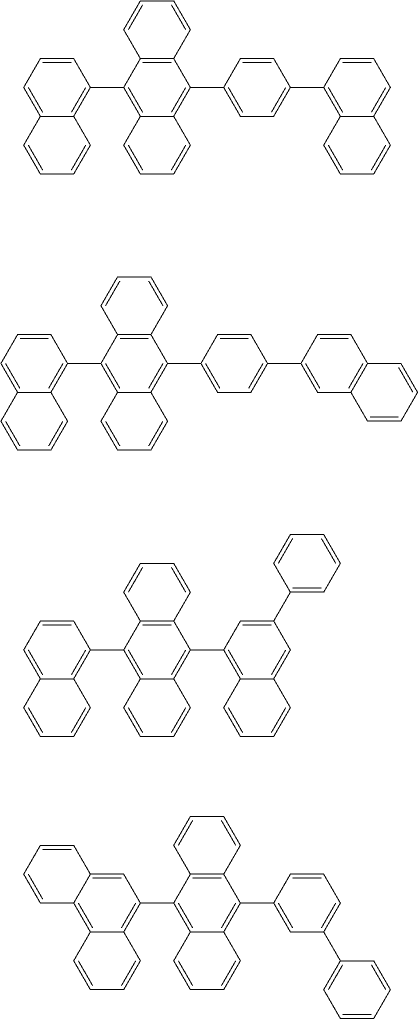

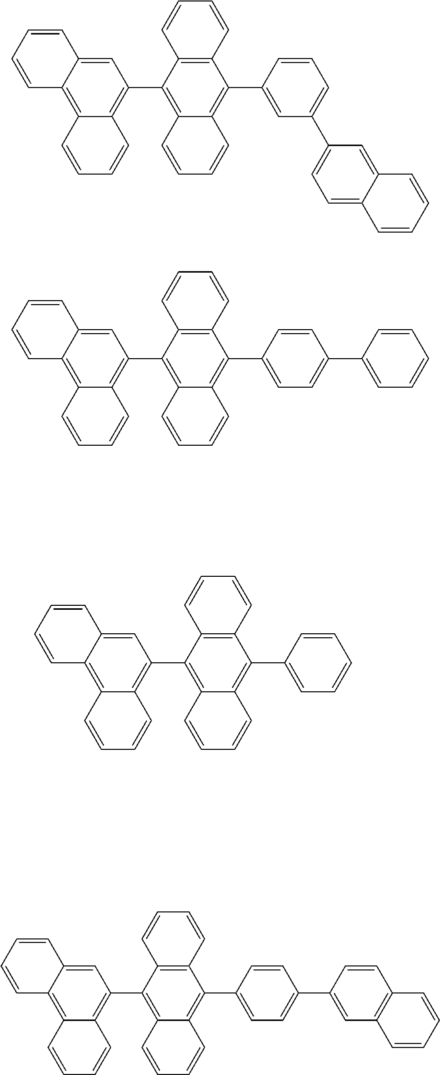

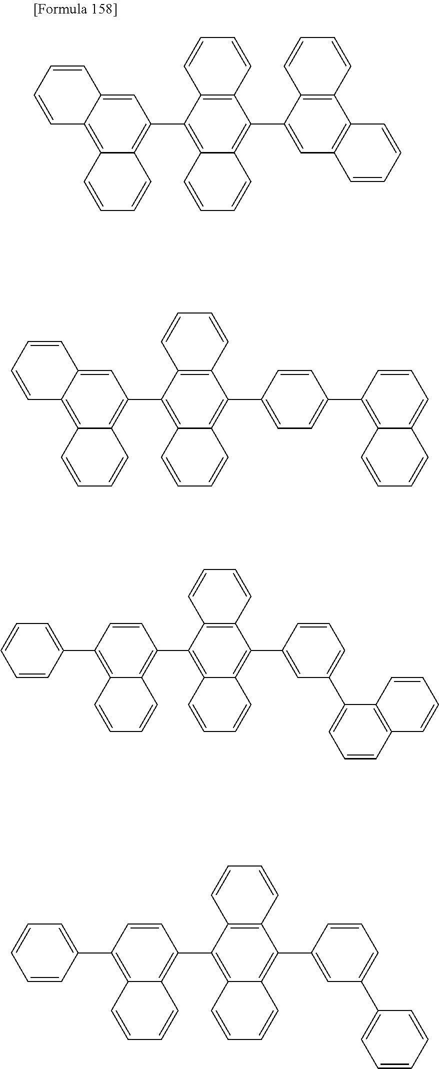

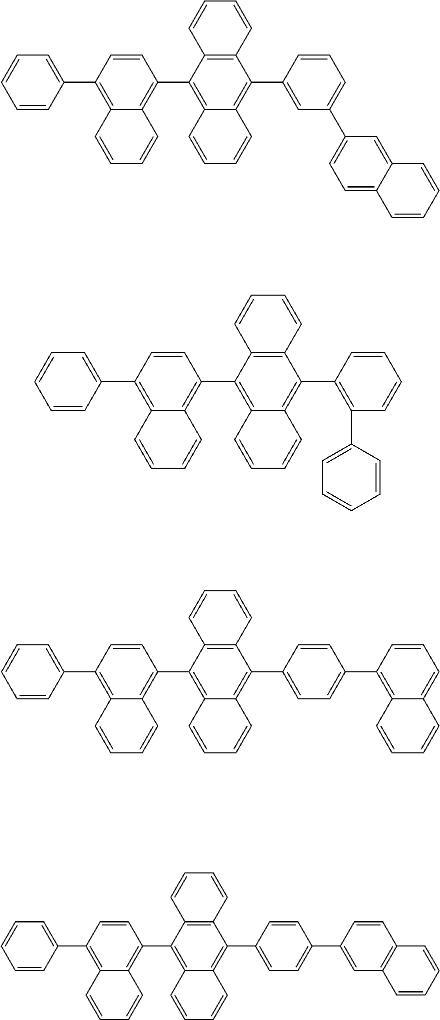

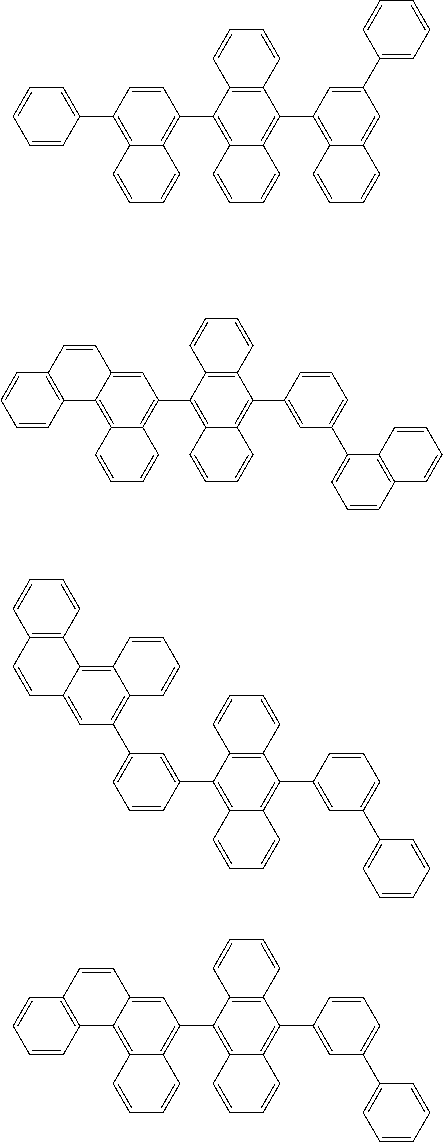

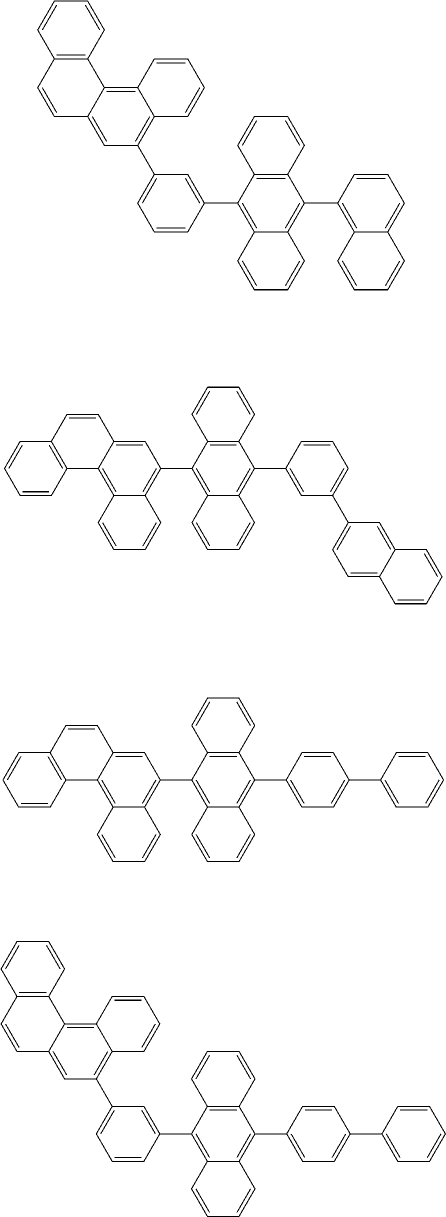

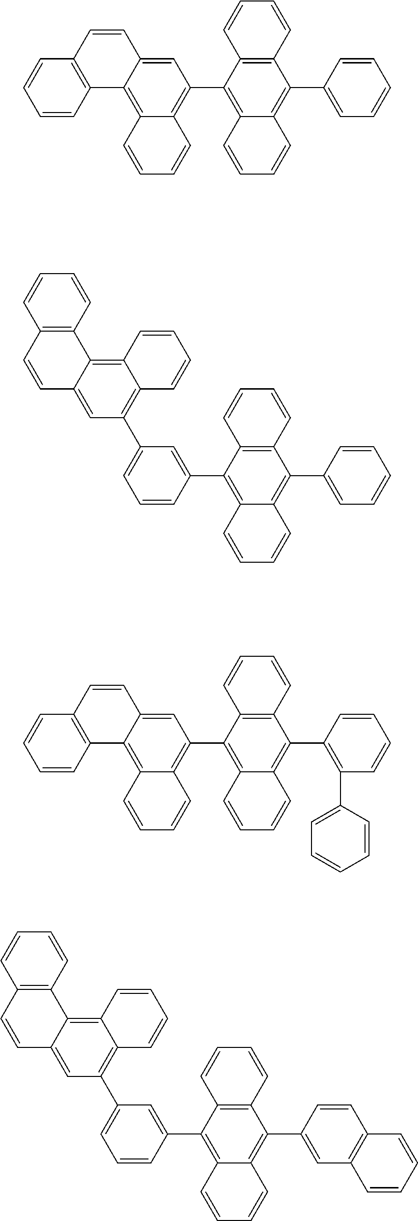

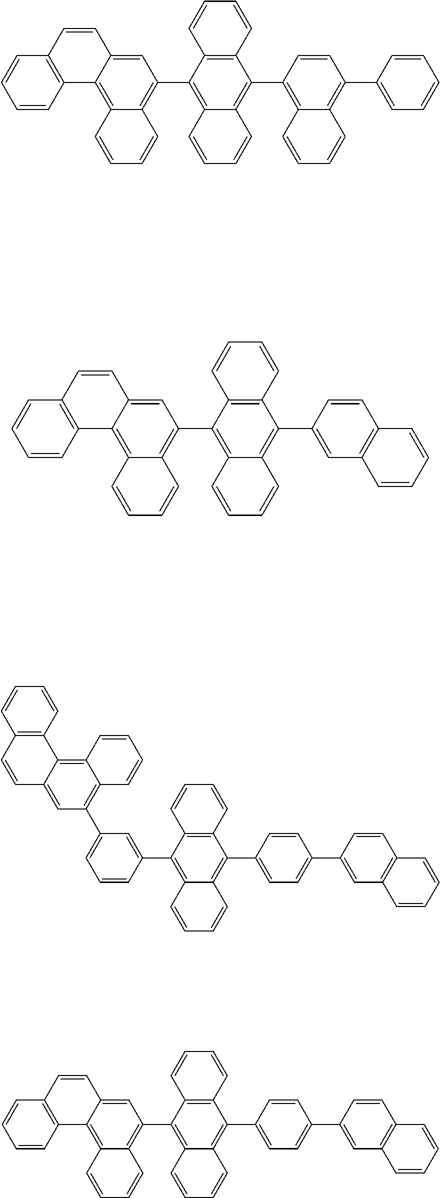

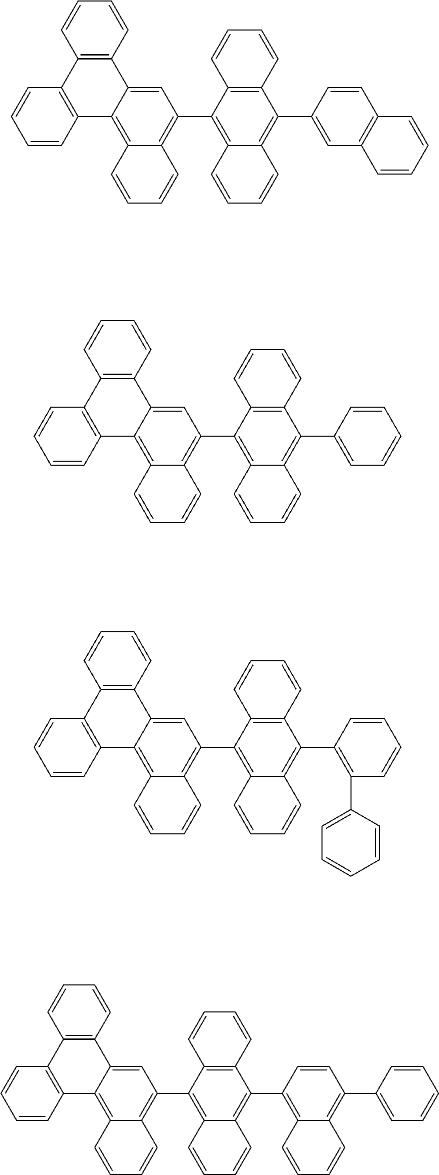

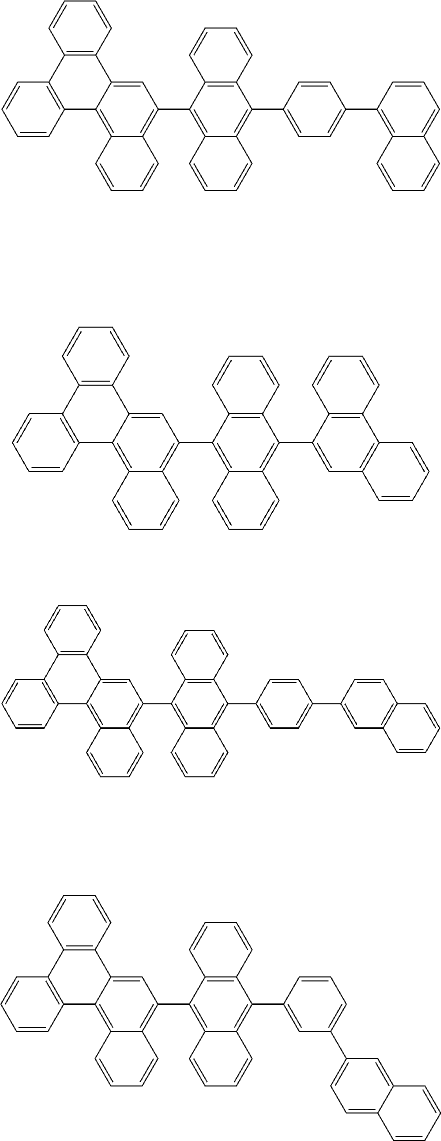

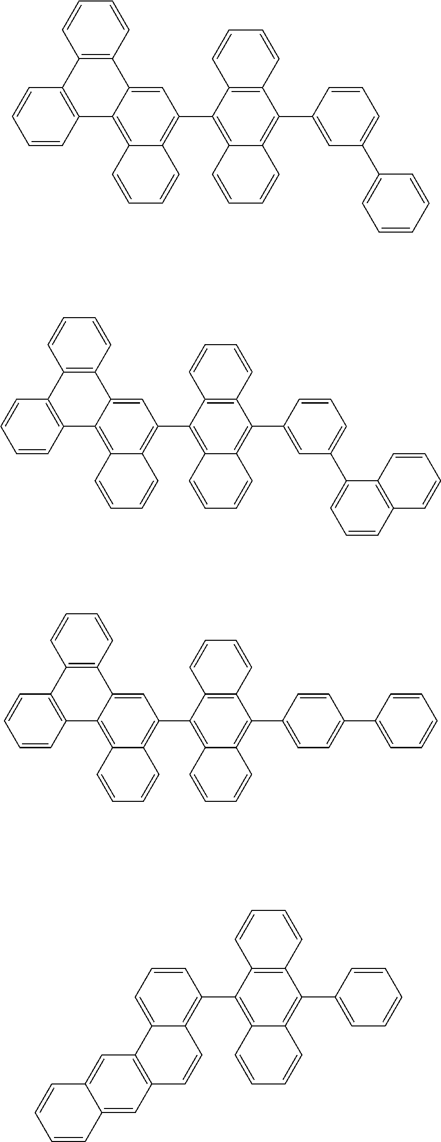

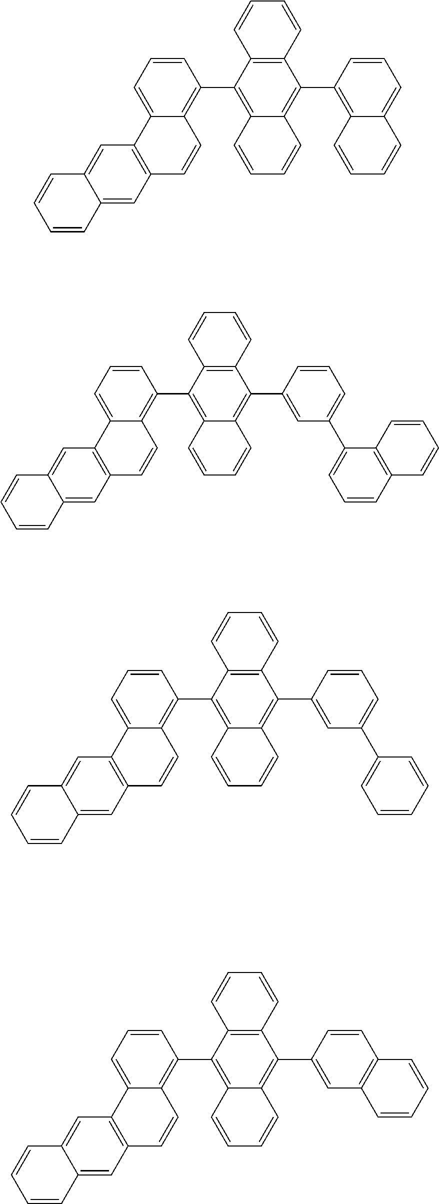

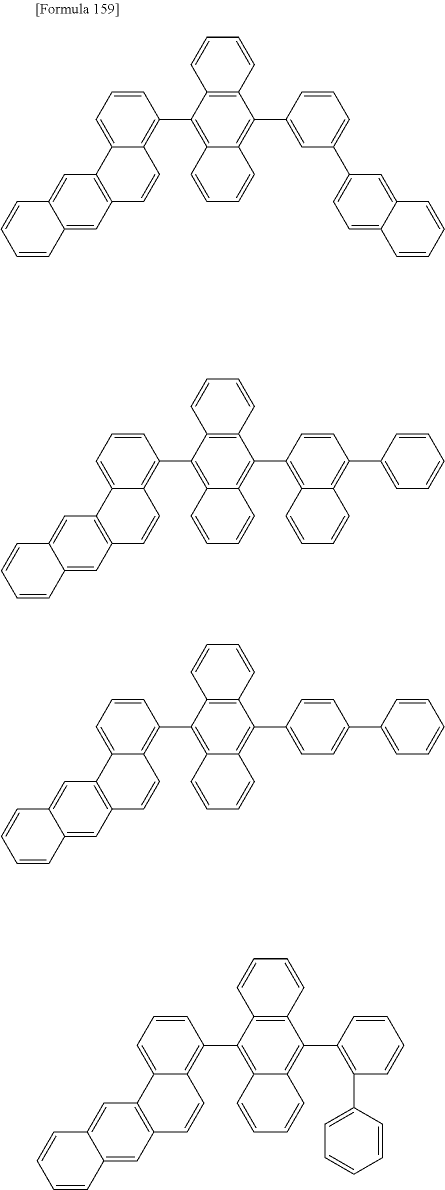

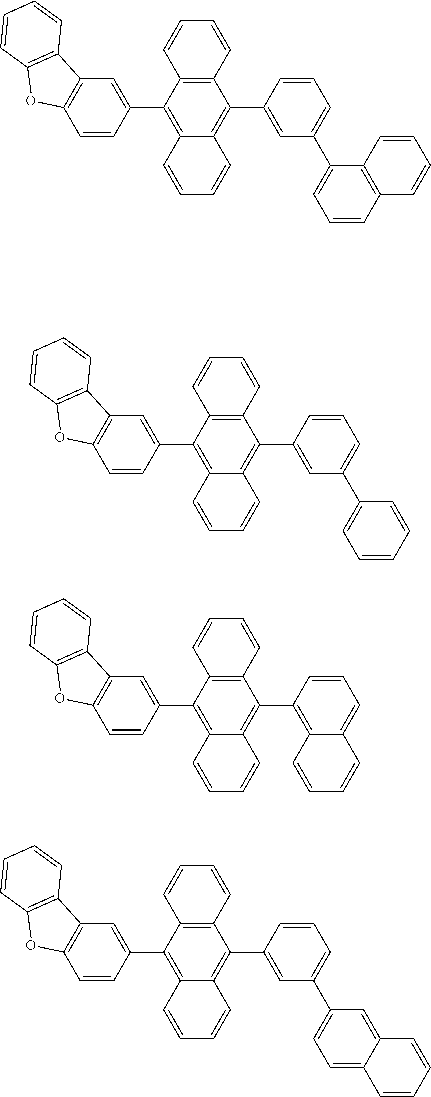 [SMILES: c1cc(-c2cccc3ccccc23)cc(-c2c3ccccc3c(-c3ccc4oc5ccccc5c4c3)c3ccccc23)c1 ; c1ccc(-c2cccc(-c3c4ccccc4c(-c4ccc5oc6ccccc6c5c4)c4ccccc34)c2)cc1 ; c1ccc2c(-c3c4ccccc4c(-c4ccc5oc6ccccc6c5c4)c4ccccc34)cccc2c1 ; c1cc(-c2ccc3ccccc3c2)cc(-c2c3ccccc3c(-c3ccc4oc5ccccc5c4c3)c3ccccc23)c1]

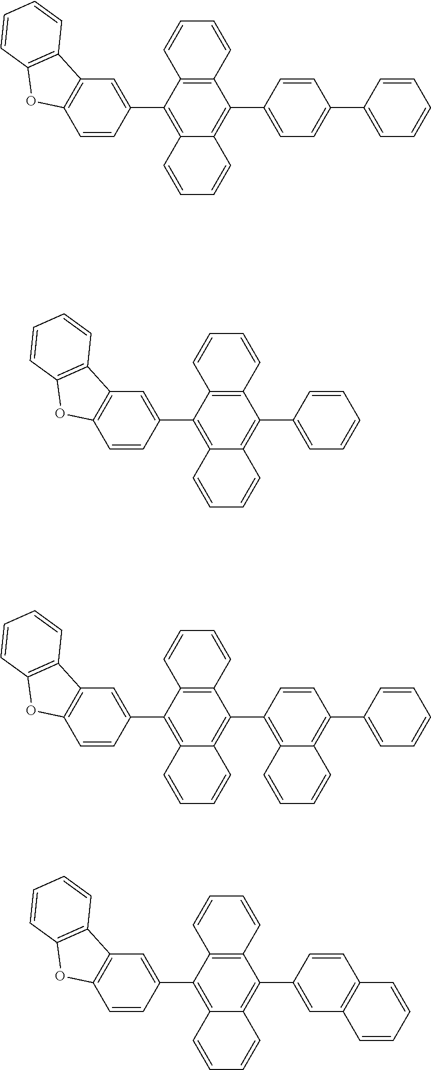 [SMILES: c1ccc(-c2ccc(-c3c4ccccc4c(-c4ccc5oc6ccccc6c5c4)c4ccccc34)cc2)cc1 ; c1ccc(-c2c3ccccc3c(-c3ccc4oc5ccccc5c4c3)c3ccccc23)cc1 ; c1ccc(-c2ccc(-c3c4ccccc4c(-c4ccc5oc6ccccc6c5c4)c4ccccc34)c3ccccc23)cc1 ; c1ccc2cc(-c3c4ccccc4c(-c4ccc5oc6ccccc6c5c4)c4ccccc34)ccc2c1]

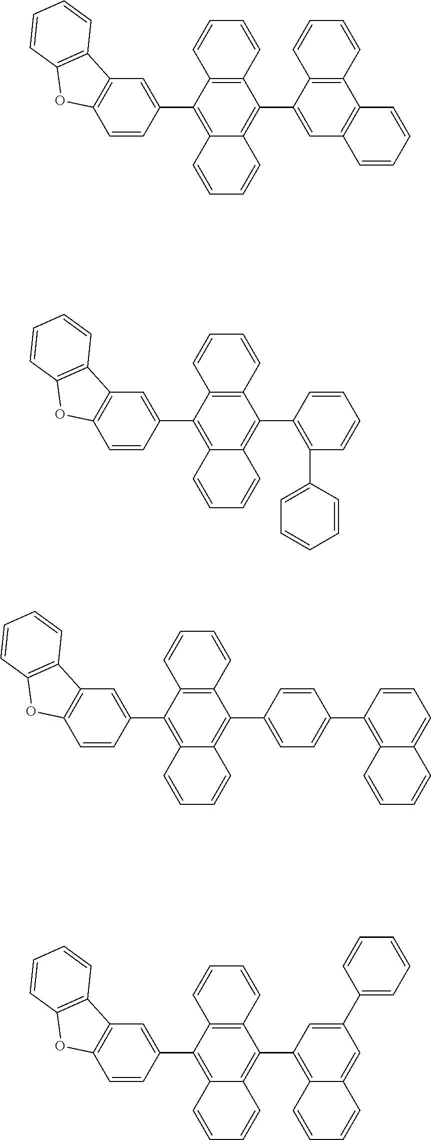 [SMILES: c1ccc2c(c1)cc(-c1c3ccccc3c(-c3ccc4oc5ccccc5c4c3)c3ccccc13)c1ccccc12 ; c1ccc(-c2ccccc2-c2c3ccccc3c(-c3ccc4oc5ccccc5c4c3)c3ccccc23)cc1 ; c1ccc2c(-c3ccc(-c4c5ccccc5c(-c5ccc6oc7ccccc7c6c5)c5ccccc45)cc3)cccc2c1 ; c1ccc(-c2cc(-c3c4ccccc4c(-c4ccc5oc6ccccc6c5c4)c4ccccc34)c3ccccc3c2)cc1]

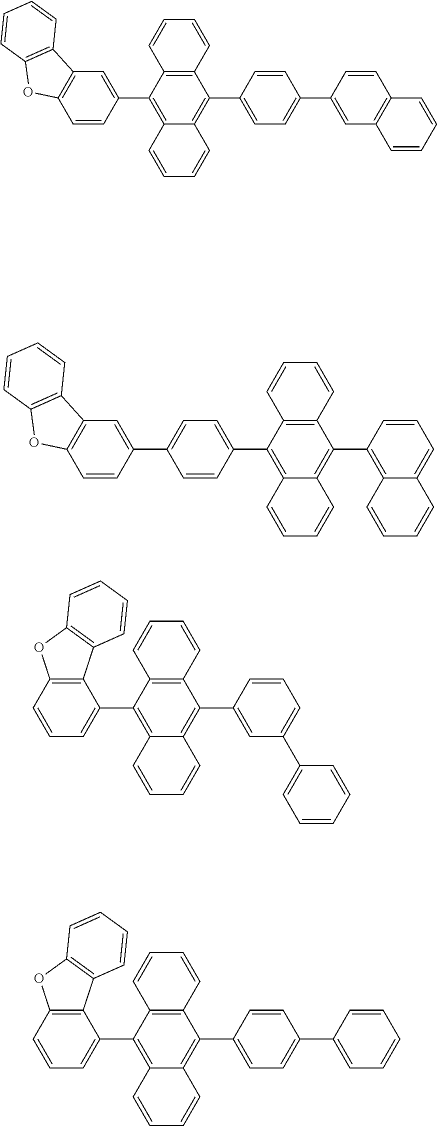 [SMILES: c1ccc2cc(-c3ccc(-c4c5ccccc5c(-c5ccc6oc7ccccc7c6c5)c5ccccc45)cc3)ccc2c1 ; c1ccc2c(-c3c4ccccc4c(-c4ccc(-c5ccc6oc7ccccc7c6c5)cc4)c4ccccc34)cccc2c1 ; c1ccc(-c2cccc(-c3c4ccccc4c(-c4cccc5oc6ccccc6c45)c4ccccc34)c2)cc1 ; c1ccc(-c2ccc(-c3c4ccccc4c(-c4cccc5oc6ccccc6c45)c4ccccc34)cc2)cc1]

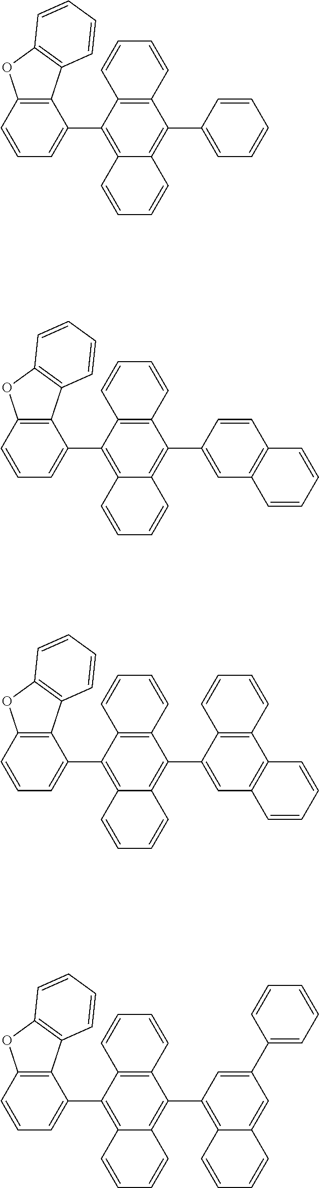 [SMILES: c1ccc(-c2c3ccccc3c(-c3cccc4oc5ccccc5c34)c3ccccc23)cc1 ; c1ccc2cc(-c3c4ccccc4c(-c4cccc5oc6ccccc6c45)c4ccccc34)ccc2c1 ; c1ccc2c(c1)cc(-c1c3ccccc3c(-c3cccc4oc5ccccc5c34)c3ccccc13)c1ccccc12 ; c1ccc(-c2cc(-c3c4ccccc4c(-c4cccc5oc6ccccc6c45)c4ccccc34)c3ccccc3c2)cc1]

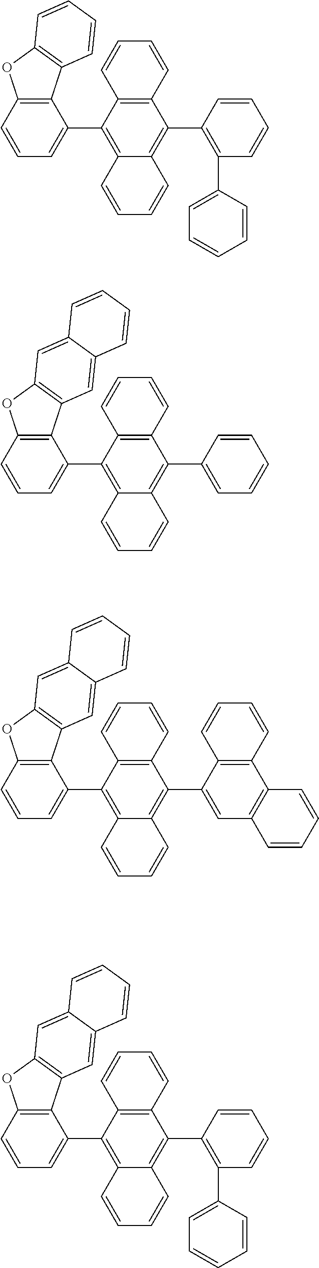 [SMILES: c1ccc(-c2ccccc2-c2c3ccccc3c(-c3cccc4oc5ccccc5c34)c3ccccc23)cc1 ; c1ccc(-c2c3ccccc3c(-c3cccc4oc5cc6ccccc6cc5c34)c3ccccc23)cc1 ; c1ccc2cc3c(cc2c1)oc1cccc(-c2c4ccccc4c(-c4cc5ccccc5c5ccccc45)c4ccccc24)c13 ; c1ccc(-c2ccccc2-c2c3ccccc3c(-c3cccc4oc5cc6ccccc6cc5c34)c3ccccc23)cc1]

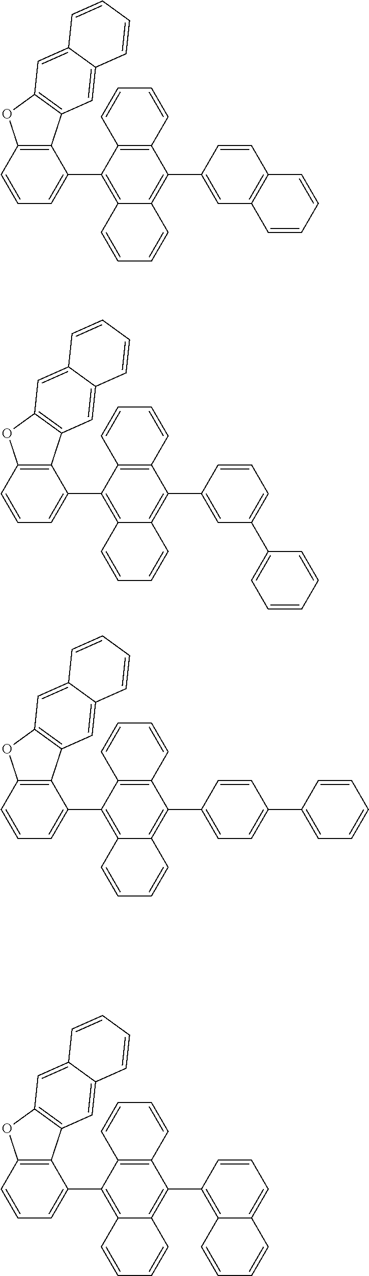 [SMILES: c1ccc2cc(-c3c4ccccc4c(-c4cccc5oc6cc7ccccc7cc6c45)c4ccccc34)ccc2c1 ; c1ccc(-c2cccc(-c3c4ccccc4c(-c4cccc5oc6cc7ccccc7cc6c45)c4ccccc34)c2)cc1 ; c1ccc(-c2ccc(-c3c4ccccc4c(-c4cccc5oc6cc7ccccc7cc6c45)c4ccccc34)cc2)cc1 ; c1ccc2cc3c(cc2c1)oc1cccc(-c2c4ccccc4c(-c4cccc5ccccc45)c4ccccc24)c13]

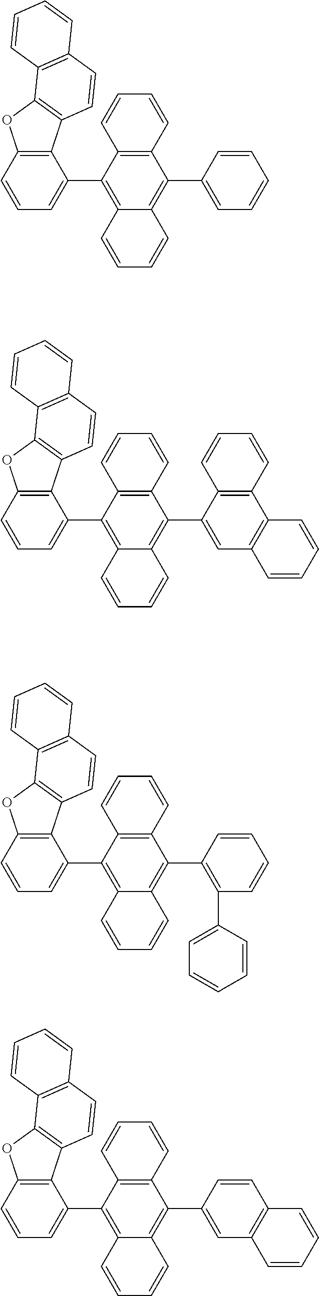 [SMILES: c1ccc(-c2c3ccccc3c(-c3cccc4oc5c6ccccc6ccc5c34)c3ccccc23)cc1 ; c1ccc2c(c1)cc(-c1c3ccccc3c(-c3cccc4oc5c6ccccc6ccc5c34)c3ccccc13)c1ccccc12 ; c1ccc(-c2ccccc2-c2c3ccccc3c(-c3cccc4oc5c6ccccc6ccc5c34)c3ccccc23)cc1 ; c1ccc2cc(-c3c4ccccc4c(-c4cccc5oc6c7ccccc7ccc6c45)c4ccccc34)ccc2c1]

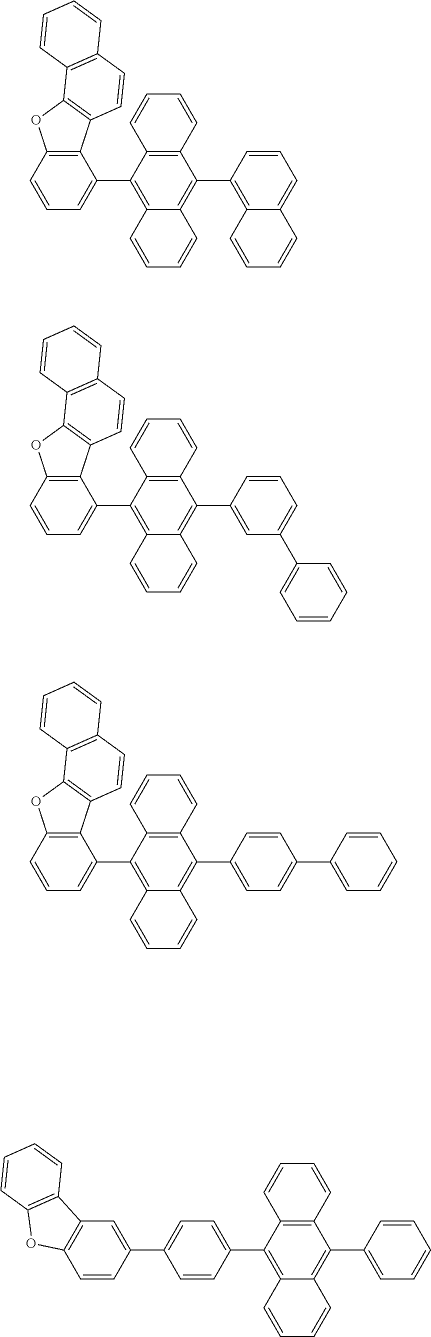 [SMILES: c1ccc2c(-c3c4ccccc4c(-c4cccc5oc6c7ccccc7ccc6c45)c4ccccc34)cccc2c1 ; c1ccc(-c2cccc(-c3c4ccccc4c(-c4cccc5oc6c7ccccc7ccc6c45)c4ccccc34)c2)cc1 ; c1ccc(-c2ccc(-c3c4ccccc4c(-c4cccc5oc6c7ccccc7ccc6c45)c4ccccc34)cc2)cc1 ; c1ccc(-c2c3ccccc3c(-c3ccc(-c4ccc5oc6ccccc6c5c4)cc3)c3ccccc23)cc1]

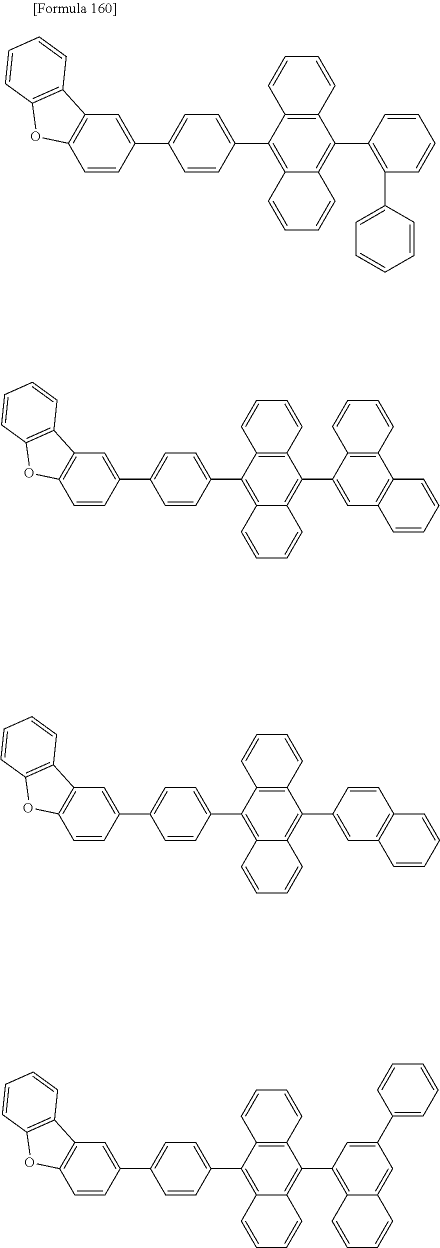 [SMILES: c1ccc(-c2ccccc2-c2c3ccccc3c(-c3ccc(-c4ccc5oc6ccccc6c5c4)cc3)c3ccccc23)cc1 ; c1ccc2c(c1)cc(-c1c3ccccc3c(-c3ccc(-c4ccc5oc6ccccc6c5c4)cc3)c3ccccc13)c1ccccc12 ; c1ccc2cc(-c3c4ccccc4c(-c4ccc(-c5ccc6oc7ccccc7c6c5)cc4)c4ccccc34)ccc2c1 ; c1ccc(-c2cc(-c3c4ccccc4c(-c4ccc(-c5ccc6oc7ccccc7c6c5)cc4)c4ccccc34)c3ccccc3c2)cc1]

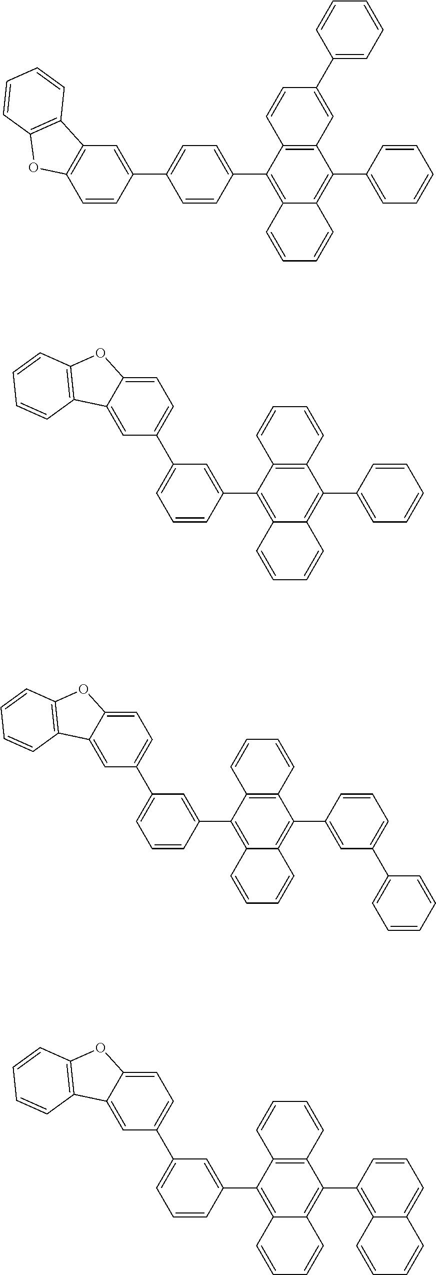 [SMILES: c1ccc(-c2ccc3c(-c4ccc(-c5ccc6oc7ccccc7c6c5)cc4)c4ccccc4c(-c4ccccc4)c3c2)cc1 ; c1ccc(-c2c3ccccc3c(-c3cccc(-c4ccc5oc6ccccc6c5c4)c3)c3ccccc23)cc1 ; c1ccc(-c2cccc(-c3c4ccccc4c(-c4cccc(-c5ccc6oc7ccccc7c6c5)c4)c4ccccc34)c2)cc1 ; c1cc(-c2ccc3oc4ccccc4c3c2)cc(-c2c3ccccc3c(-c3cccc4ccccc34)c3ccccc23)c1]

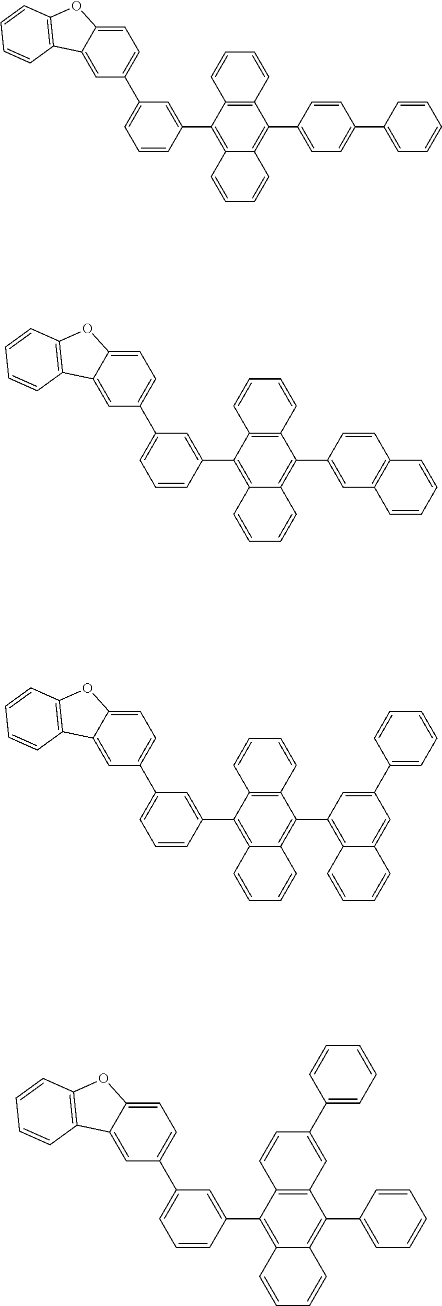 [SMILES: c1ccc(-c2ccc(-c3c4ccccc4c(-c4cccc(-c5ccc6oc7ccccc7c6c5)c4)c4ccccc34)cc2)cc1 ; c1cc(-c2ccc3oc4ccccc4c3c2)cc(-c2c3ccccc3c(-c3ccc4ccccc4c3)c3ccccc23)c1 ; c1ccc(-c2cc(-c3c4ccccc4c(-c4cccc(-c5ccc6oc7ccccc7c6c5)c4)c4ccccc34)c3ccccc3c2)cc1 ; c1ccc(-c2ccc3c(-c4cccc(-c5ccc6oc7ccccc7c6c5)c4)c4ccccc4c(-c4ccccc4)c3c2)cc1]

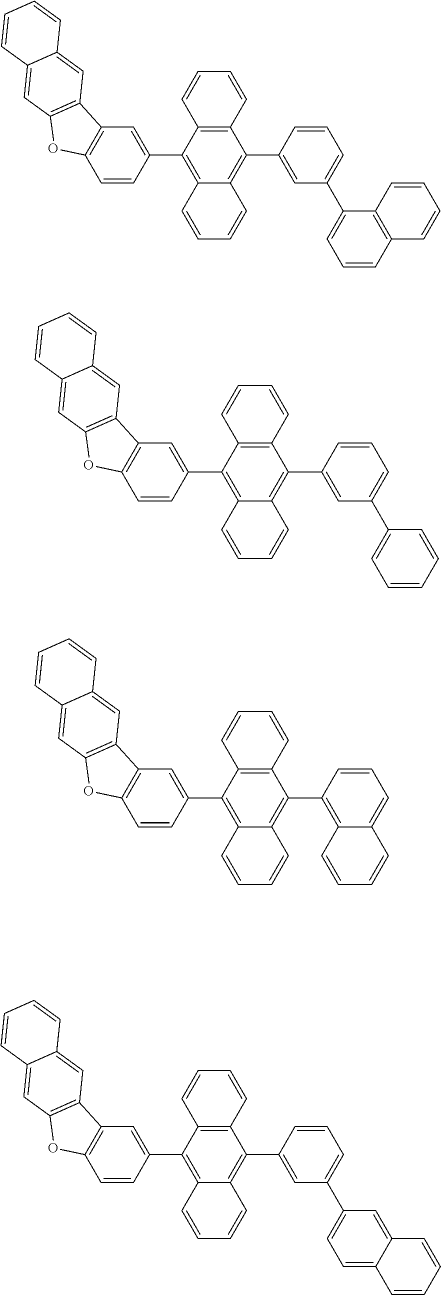 [SMILES: c1cc(-c2cccc3ccccc23)cc(-c2c3ccccc3c(-c3ccc4oc5cc6ccccc6cc5c4c3)c3ccccc23)c1 ; c1ccc(-c2cccc(-c3c4ccccc4c(-c4ccc5oc6cc7ccccc7cc6c5c4)c4ccccc34)c2)cc1 ; c1ccc2cc3c(cc2c1)oc1ccc(-c2c4ccccc4c(-c4cccc5ccccc45)c4ccccc24)cc13 ; c1cc(-c2ccc3ccccc3c2)cc(-c2c3ccccc3c(-c3ccc4oc5cc6ccccc6cc5c4c3)c3ccccc23)c1]

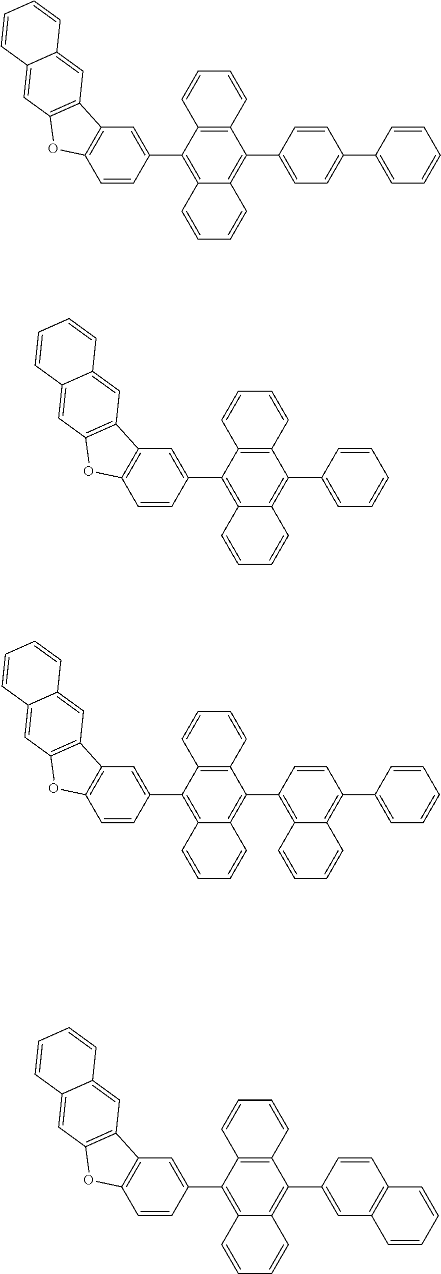 [SMILES: c1ccc(-c2ccc(-c3c4ccccc4c(-c4ccc5oc6cc7ccccc7cc6c5c4)c4ccccc34)cc2)cc1 ; c1ccc(-c2c3ccccc3c(-c3ccc4oc5cc6ccccc6cc5c4c3)c3ccccc23)cc1 ; c1ccc(-c2ccc(-c3c4ccccc4c(-c4ccc5oc6cc7ccccc7cc6c5c4)c4ccccc34)c3ccccc23)cc1 ; c1ccc2cc(-c3c4ccccc4c(-c4ccc5oc6cc7ccccc7cc6c5c4)c4ccccc34)ccc2c1]

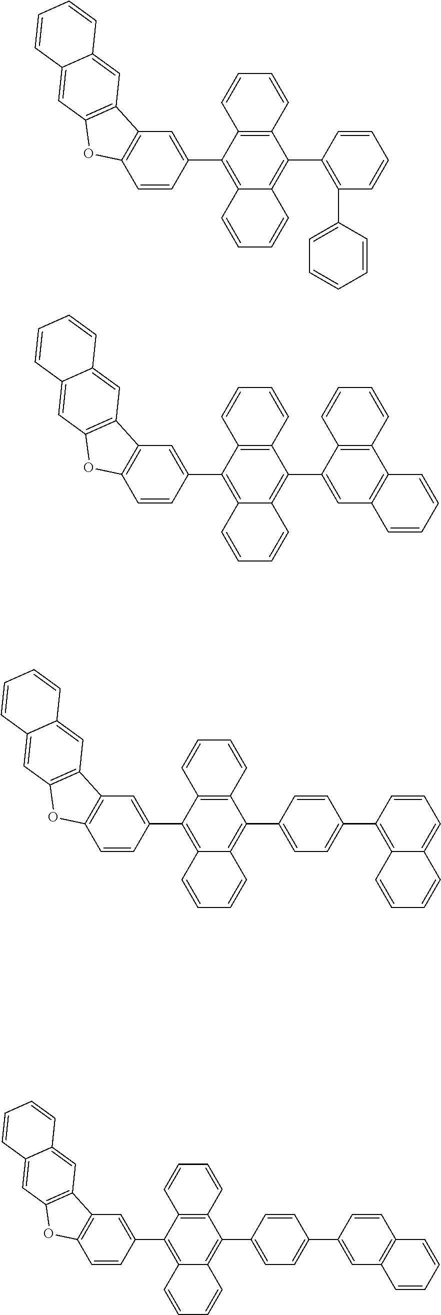 [SMILES: c1ccc(-c2ccccc2-c2c3ccccc3c(-c3ccc4oc5cc6ccccc6cc5c4c3)c3ccccc23)cc1 ; c1ccc2cc3c(cc2c1)oc1ccc(-c2c4ccccc4c(-c4cc5ccccc5c5ccccc45)c4ccccc24)cc13 ; c1ccc2cc3c(cc2c1)oc1ccc(-c2c4ccccc4c(-c4ccc(-c5cccc6ccccc56)cc4)c4ccccc24)cc13 ; c1ccc2cc(-c3ccc(-c4c5ccccc5c(-c5ccc6oc7cc8ccccc8cc7c6c5)c5ccccc45)cc3)ccc2c1]

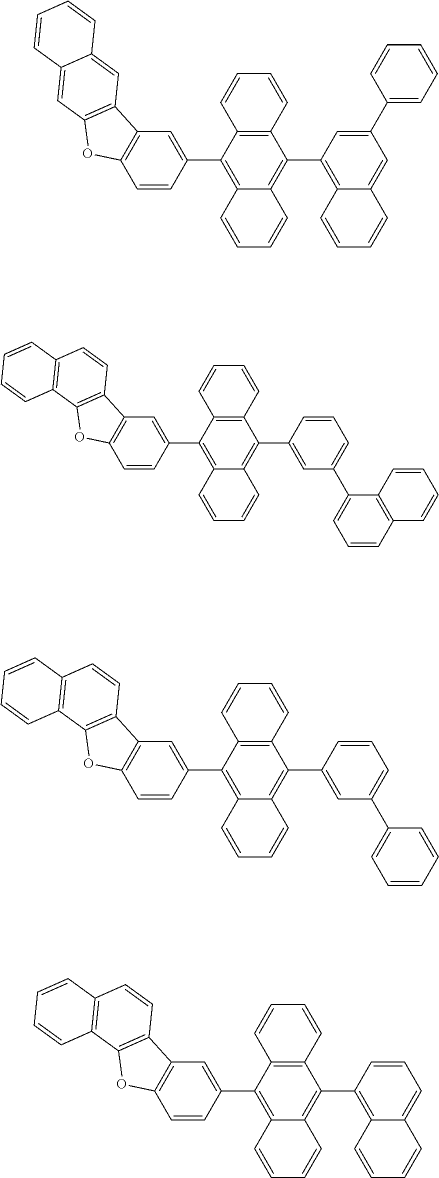 [SMILES: c1ccc(-c2cc(-c3c4ccccc4c(-c4ccc5oc6cc7ccccc7cc6c5c4)c4ccccc34)c3ccccc3c2)cc1 ; c1cc(-c2cccc3ccccc23)cc(-c2c3ccccc3c(-c3ccc4oc5c6ccccc6ccc5c4c3)c3ccccc23)c1 ; c1ccc(-c2cccc(-c3c4ccccc4c(-c4ccc5oc6c7ccccc7ccc6c5c4)c4ccccc34)c2)cc1 ; c1ccc2c(-c3c4ccccc4c(-c4ccc5oc6c7ccccc7ccc6c5c4)c4ccccc34)cccc2c1]

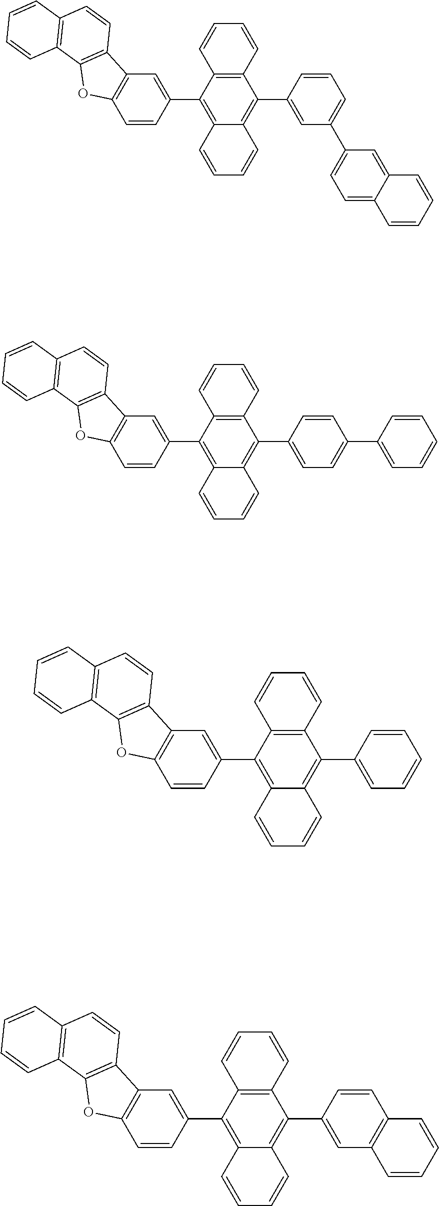 [SMILES: c1cc(-c2ccc3ccccc3c2)cc(-c2c3ccccc3c(-c3ccc4oc5c6ccccc6ccc5c4c3)c3ccccc23)c1 ; c1ccc(-c2ccc(-c3c4ccccc4c(-c4ccc5oc6c7ccccc7ccc6c5c4)c4ccccc34)cc2)cc1 ; c1ccc(-c2c3ccccc3c(-c3ccc4oc5c6ccccc6ccc5c4c3)c3ccccc23)cc1 ; c1ccc2cc(-c3c4ccccc4c(-c4ccc5oc6c7ccccc7ccc6c5c4)c4ccccc34)ccc2c1]

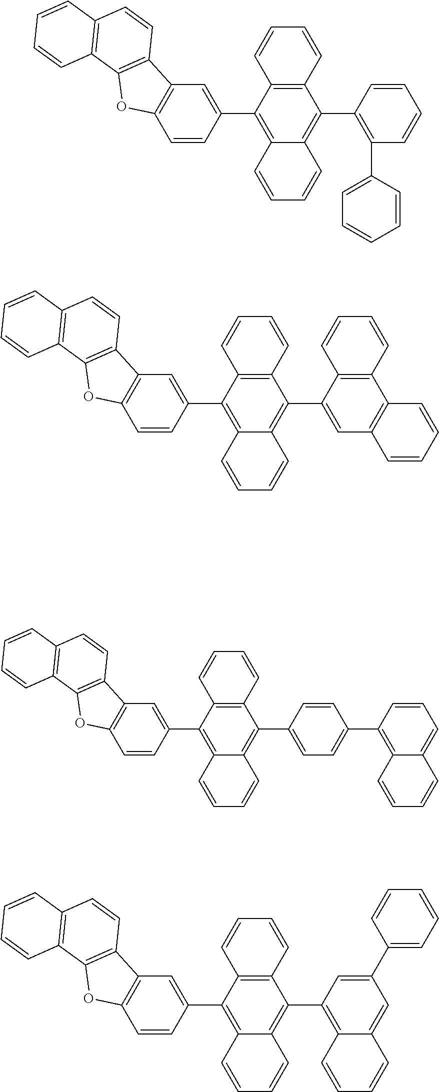 [SMILES: c1ccc(-c2ccccc2-c2c3ccccc3c(-c3ccc4oc5c6ccccc6ccc5c4c3)c3ccccc23)cc1 ; c1ccc2c(c1)cc(-c1c3ccccc3c(-c3ccc4oc5c6ccccc6ccc5c4c3)c3ccccc13)c1ccccc12 ; c1ccc2c(-c3ccc(-c4c5ccccc5c(-c5ccc6oc7c8ccccc8ccc7c6c5)c5ccccc45)cc3)cccc2c1 ; c1ccc(-c2cc(-c3c4ccccc4c(-c4ccc5oc6c7ccccc7ccc6c5c4)c4ccccc34)c3ccccc3c2)cc1]

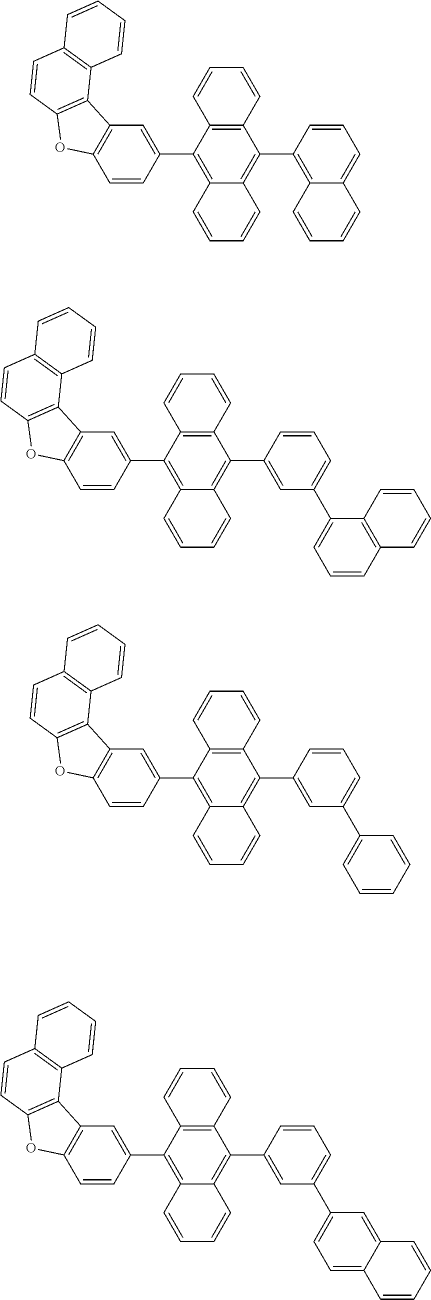 [SMILES: c1ccc2c(-c3c4ccccc4c(-c4ccc5oc6ccc7ccccc7c6c5c4)c4ccccc34)cccc2c1 ; c1cc(-c2cccc3ccccc23)cc(-c2c3ccccc3c(-c3ccc4oc5ccc6ccccc6c5c4c3)c3ccccc23)c1 ; c1ccc(-c2cccc(-c3c4ccccc4c(-c4ccc5oc6ccc7ccccc7c6c5c4)c4ccccc34)c2)cc1 ; c1cc(-c2ccc3ccccc3c2)cc(-c2c3ccccc3c(-c3ccc4oc5ccc6ccccc6c5c4c3)c3ccccc23)c1]

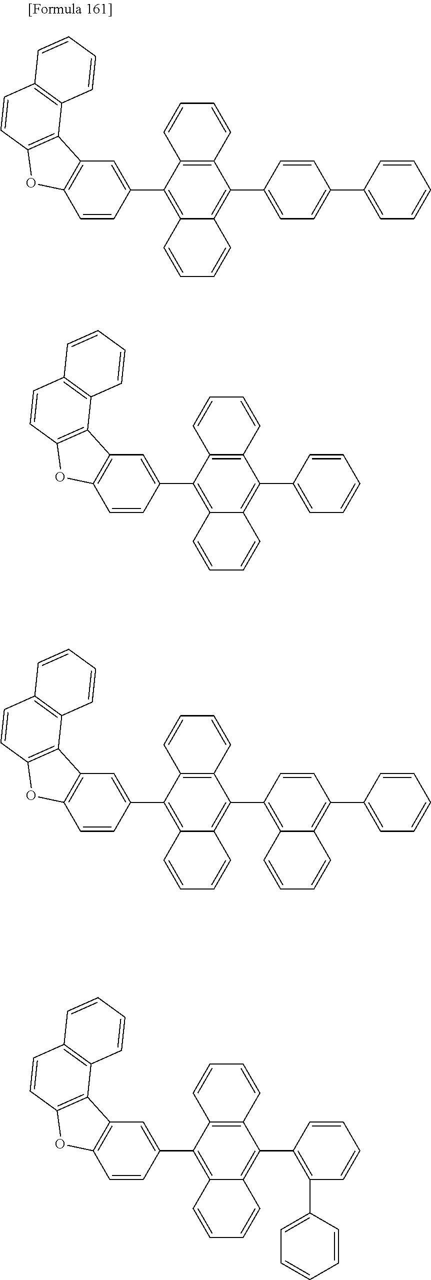 [SMILES: c1ccc(-c2ccc(-c3c4ccccc4c(-c4ccc5oc6ccc7ccccc7c6c5c4)c4ccccc34)cc2)cc1 ; c1ccc(-c2c3ccccc3c(-c3ccc4oc5ccc6ccccc6c5c4c3)c3ccccc23)cc1 ; c1ccc(-c2ccc(-c3c4ccccc4c(-c4ccc5oc6ccc7ccccc7c6c5c4)c4ccccc34)c3ccccc23)cc1 ; c1ccc(-c2ccccc2-c2c3ccccc3c(-c3ccc4oc5ccc6ccccc6c5c4c3)c3ccccc23)cc1]

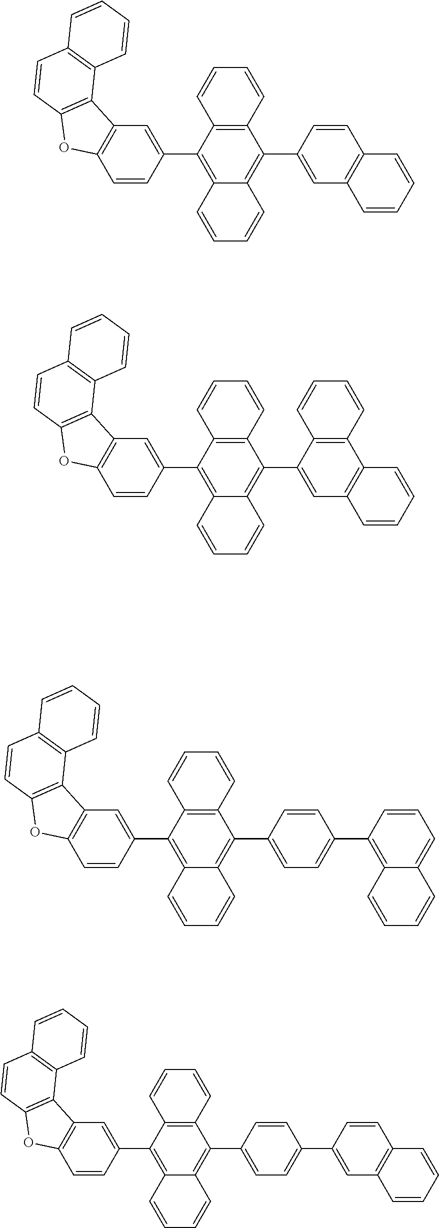 [SMILES: c1ccc2cc(-c3c4ccccc4c(-c4ccc5oc6ccc7ccccc7c6c5c4)c4ccccc34)ccc2c1 ; c1ccc2c(c1)cc(-c1c3ccccc3c(-c3ccc4oc5ccc6ccccc6c5c4c3)c3ccccc13)c1ccccc12 ; c1ccc2c(-c3ccc(-c4c5ccccc5c(-c5ccc6oc7ccc8ccccc8c7c6c5)c5ccccc45)cc3)cccc2c1 ; c1ccc2cc(-c3ccc(-c4c5ccccc5c(-c5ccc6oc7ccc8ccccc8c7c6c5)c5ccccc45)cc3)ccc2c1]

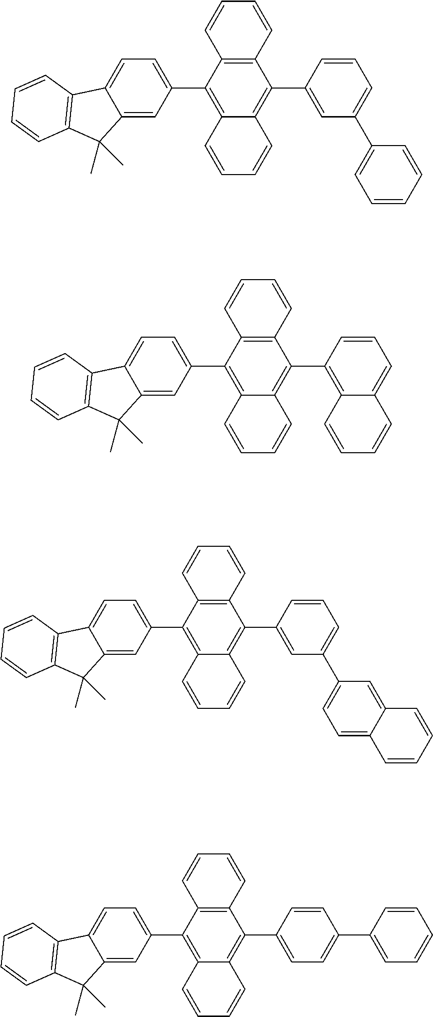

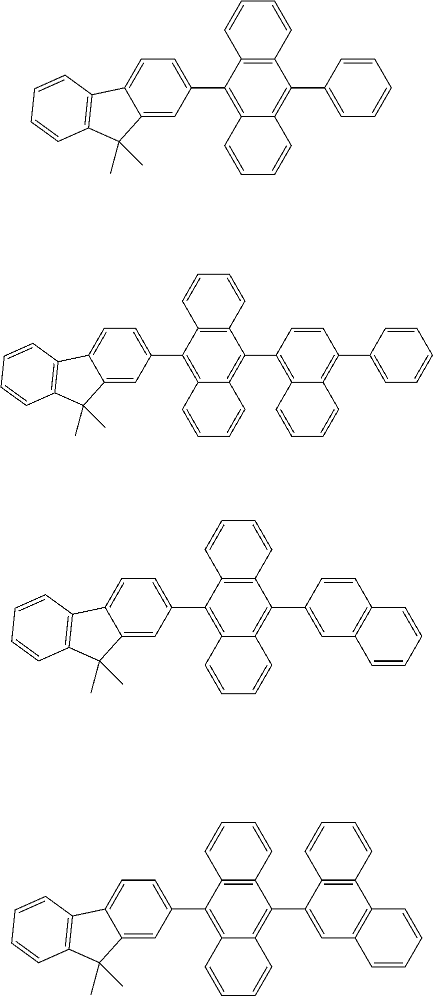

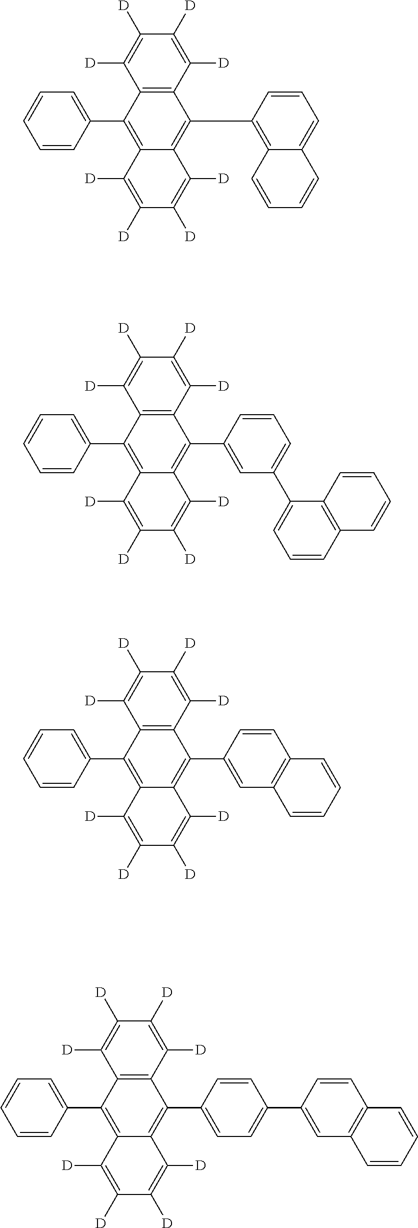 [SMILES: [2H]c1c([2H])c([2H])c2c(-c3cccc4ccccc34)c3c([2H])c([2H])c([2H])c([2H])c3c(-c3ccccc3)c2c1[2H] ; [2H]c1c([2H])c([2H])c2c(-c3cccc(-c4cccc5ccccc45)c3)c3c([2H])c([2H])c([2H])c([2H])c3c(-c3ccccc3)c2c1[2H] ; [2H]c1c([2H])c([2H])c2c(-c3ccc4ccccc4c3)c3c([2H])c([2H])c([2H])c([2H])c3c(-c3ccccc3)c2c1[2H] ; [2H]c1c([2H])c([2H])c2c(-c3ccc(-c4ccc5ccccc5c4)cc3)c3c([2H])c([2H])c([2H])c([2H])c3c(-c3ccccc3)c2c1[2H]]

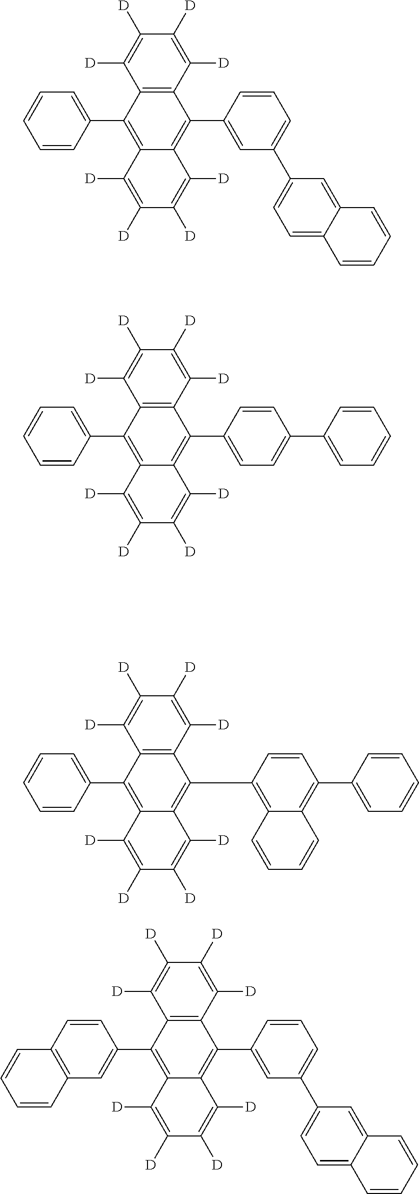

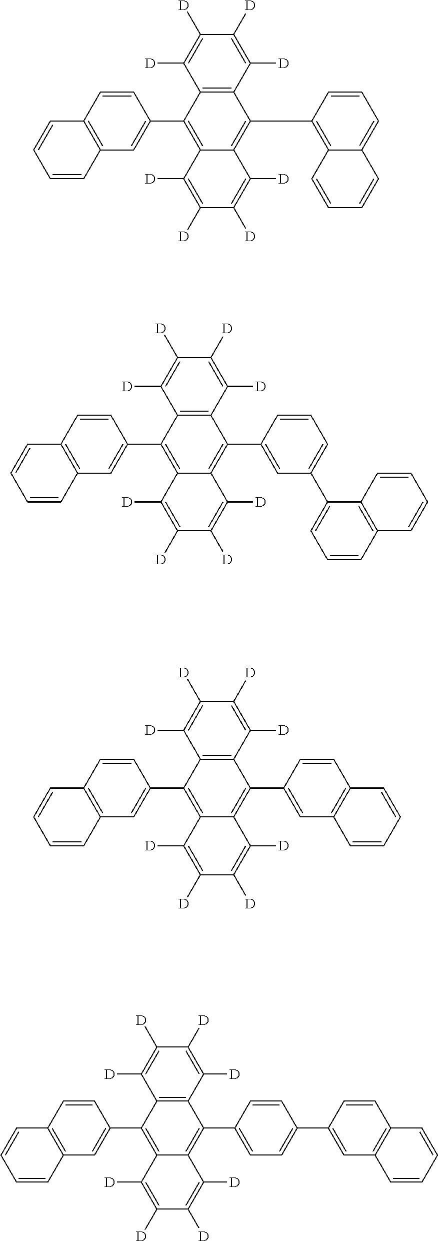 [SMILES: [2H]c1c([2H])c([2H])c2c(-c3cccc4ccccc34)c3c([2H])c([2H])c([2H])c([2H])c3c(-c3ccc4ccccc4c3)c2c1[2H] ; [2H]c1c([2H])c([2H])c2c(-c3ccc4ccccc4c3)c3c([2H])c([2H])c([2H])c([2H])c3c(-c3cccc(-c4cccc5ccccc45)c3)c2c1[2H] ; [2H]c1c([2H])c([2H])c2c(-c3ccc4ccccc4c3)c3c([2H])c([2H])c([2H])c([2H])c3c(-c3ccc4ccccc4c3)c2c1[2H] ; [2H]c1c([2H])c([2H])c2c(-c3ccc4ccccc4c3)c3c([2H])c([2H])c([2H])c([2H])c3c(-c3ccc(-c4ccc5ccccc5c4)cc3)c2c1[2H]]

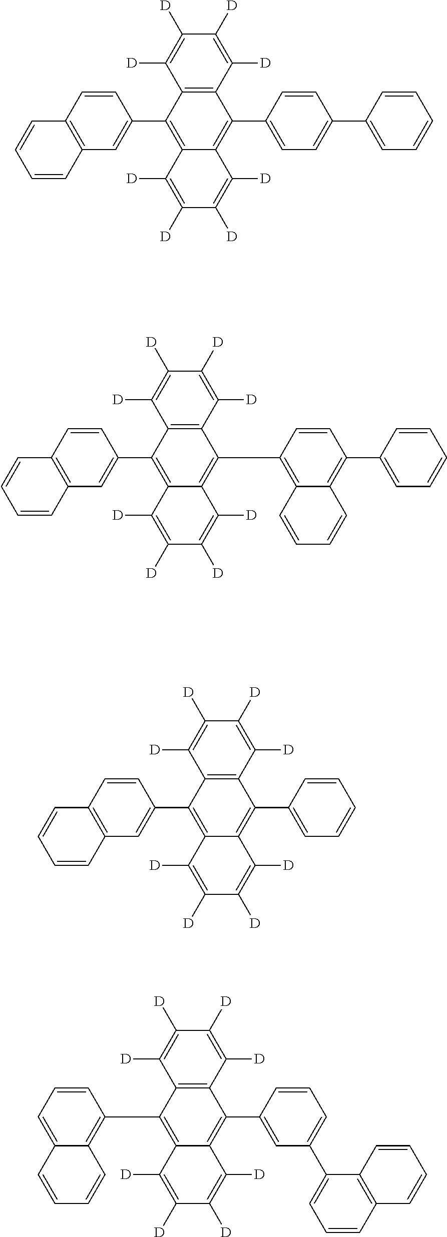 [SMILES: [2H]c1c([2H])c([2H])c2c(-c3ccc4ccccc4c3)c3c([2H])c([2H])c([2H])c([2H])c3c(-c3ccc(-c4ccccc4)cc3)c2c1[2H] ; [2H]c1c([2H])c([2H])c2c(-c3ccc(-c4ccccc4)c4ccccc34)c3c([2H])c([2H])c([2H])c([2H])c3c(-c3ccc4ccccc4c3)c2c1[2H] ; [2H]c1c([2H])c([2H])c2c(-c3ccc4ccccc4c3)c3c([2H])c([2H])c([2H])c([2H])c3c(-c3ccccc3)c2c1[2H] ; [2H]c1c([2H])c([2H])c2c(-c3cccc4ccccc34)c3c([2H])c([2H])c([2H])c([2H])c3c(-c3cccc(-c4cccc5ccccc45)c3)c2c1[2H]]

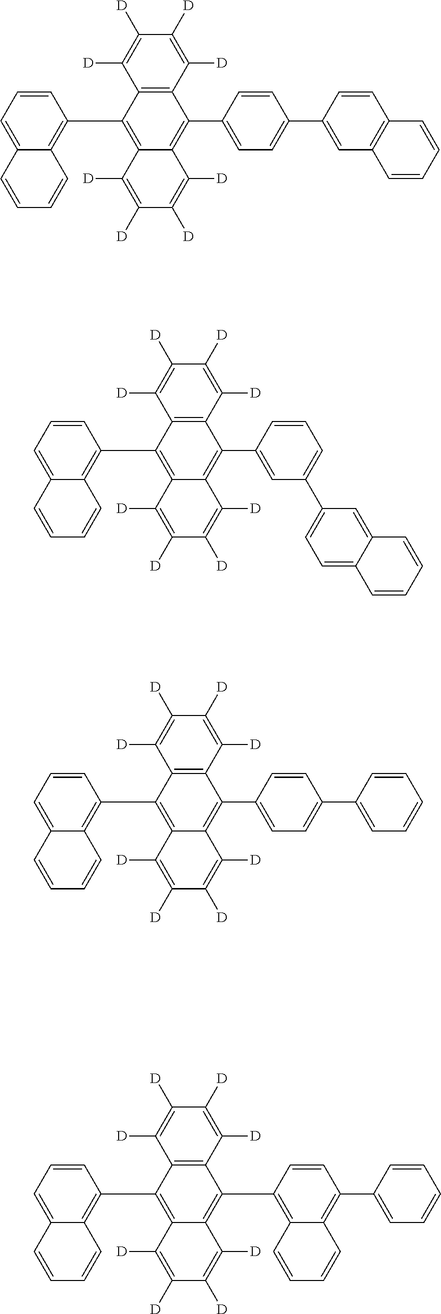 [SMILES: [2H]c1c([2H])c([2H])c2c(-c3cccc4ccccc34)c3c([2H])c([2H])c([2H])c([2H])c3c(-c3ccc(-c4ccc5ccccc5c4)cc3)c2c1[2H] ; [2H]c1c([2H])c([2H])c2c(-c3cccc4ccccc34)c3c([2H])c([2H])c([2H])c([2H])c3c(-c3cccc(-c4ccc5ccccc5c4)c3)c2c1[2H] ; [2H]c1c([2H])c([2H])c2c(-c3cccc4ccccc34)c3c([2H])c([2H])c([2H])c([2H])c3c(-c3ccc(-c4ccccc4)cc3)c2c1[2H] ; [2H]c1c([2H])c([2H])c2c(-c3ccc(-c4ccccc4)c4ccccc34)c3c([2H])c([2H])c([2H])c([2H])c3c(-c3cccc4ccccc34)c2c1[2H]]

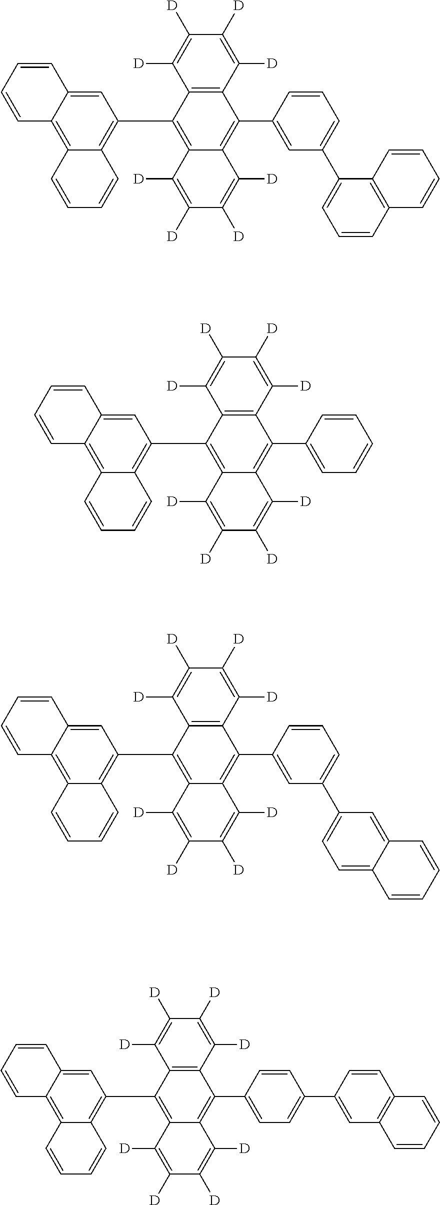 [SMILES: [2H]c1c([2H])c([2H])c2c(-c3cc4ccccc4c4ccccc34)c3c([2H])c([2H])c([2H])c([2H])c3c(-c3cccc(-c4cccc5ccccc45)c3)c2c1[2H] ; [2H]c1c([2H])c([2H])c2c(-c3cc4ccccc4c4ccccc34)c3c([2H])c([2H])c([2H])c([2H])c3c(-c3ccccc3)c2c1[2H] ; [2H]c1c([2H])c([2H])c2c(-c3cc4ccccc4c4ccccc34)c3c([2H])c([2H])c([2H])c([2H])c3c(-c3cccc(-c4ccc5ccccc5c4)c3)c2c1[2H] ; [2H]c1c([2H])c([2H])c2c(-c3cc4ccccc4c4ccccc34)c3c([2H])c([2H])c([2H])c([2H])c3c(-c3ccc(-c4ccc5ccccc5c4)cc3)c2c1[2H]]

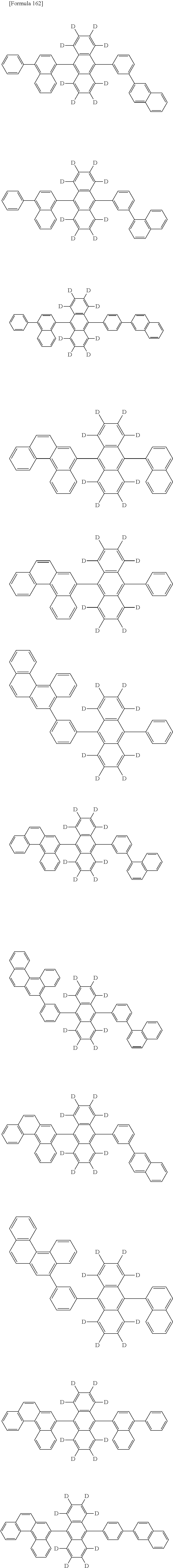

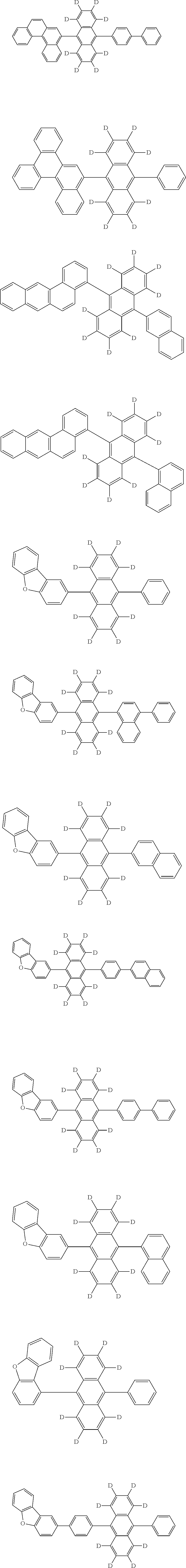

[chemical structure]

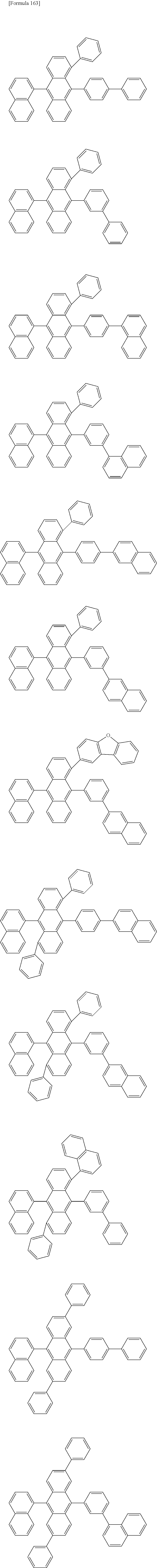

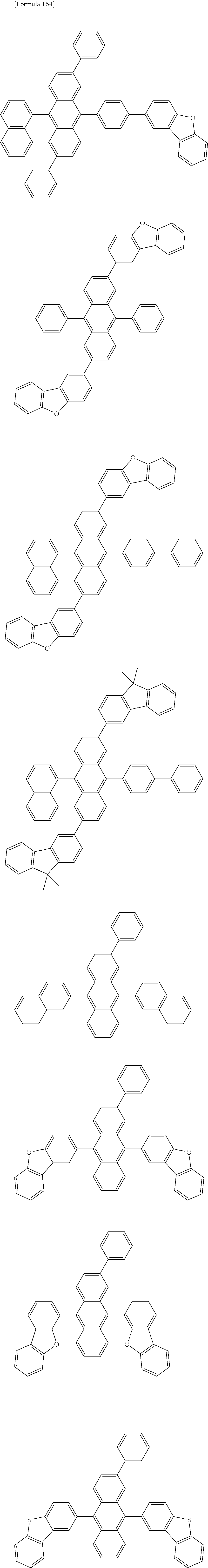

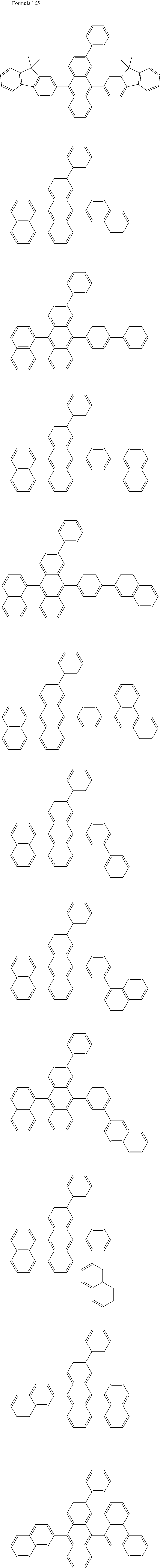

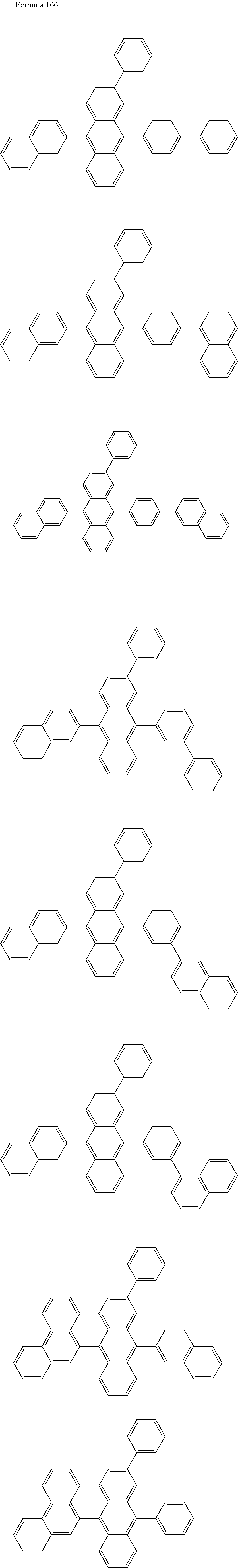

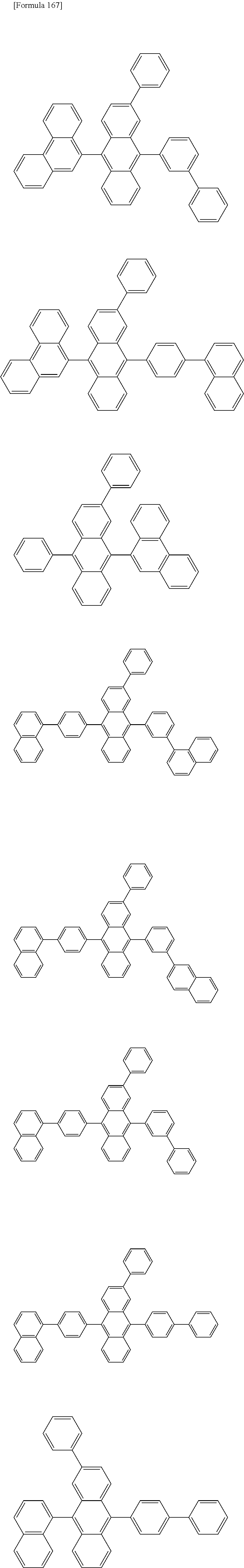

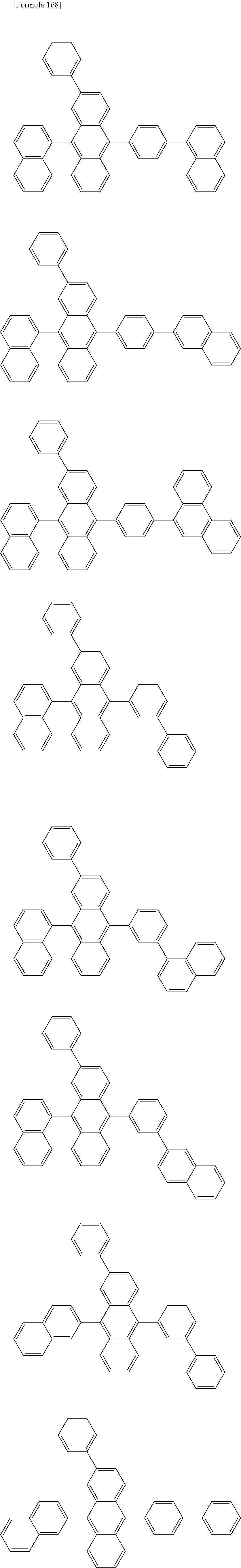

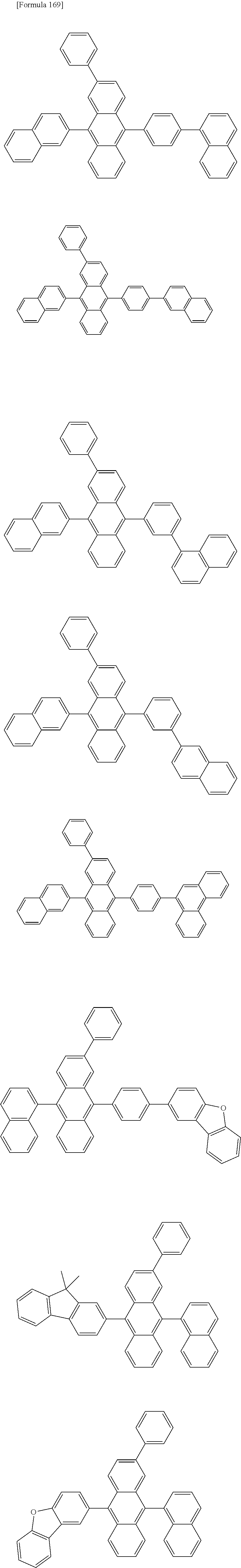

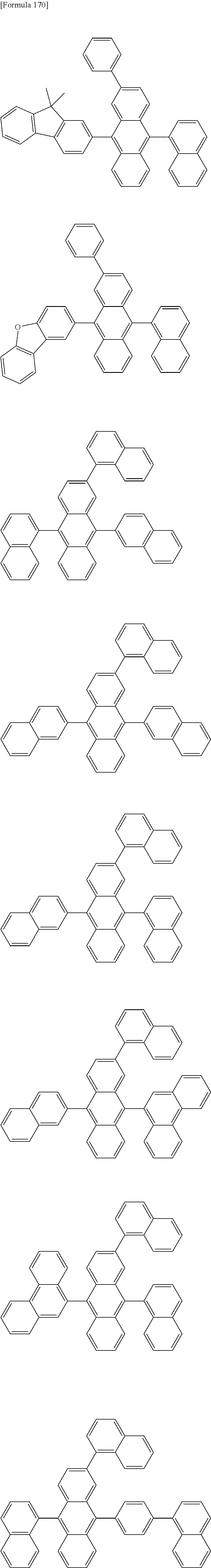

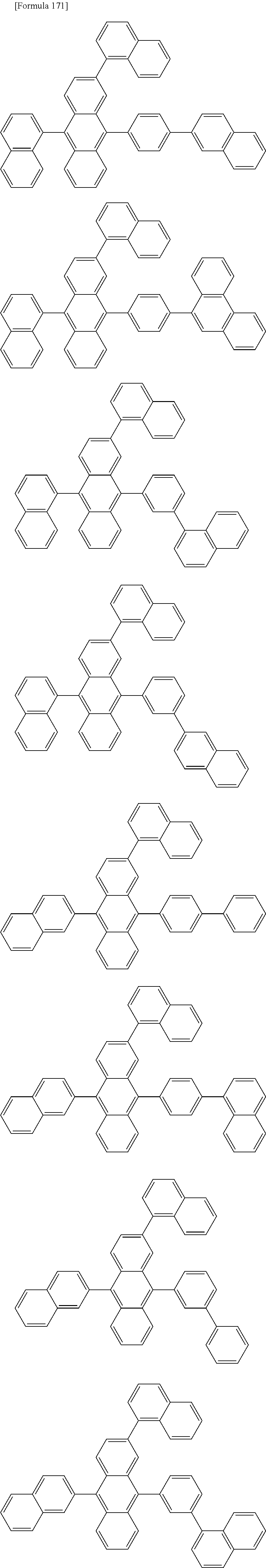

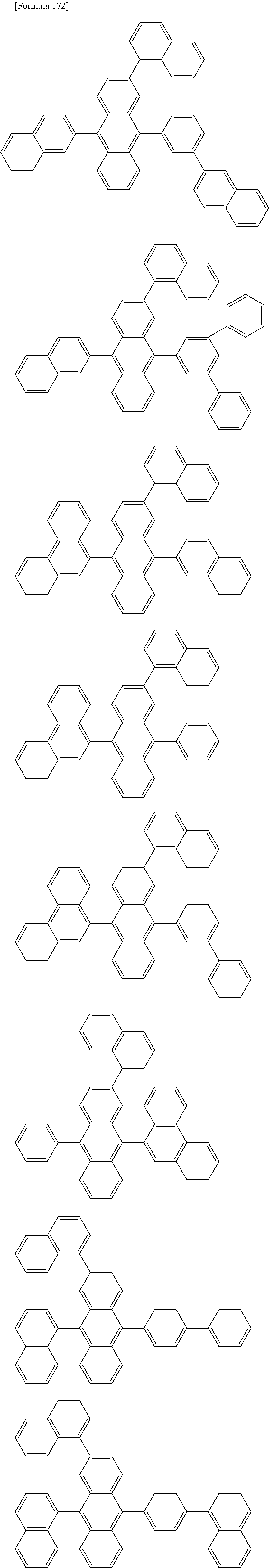

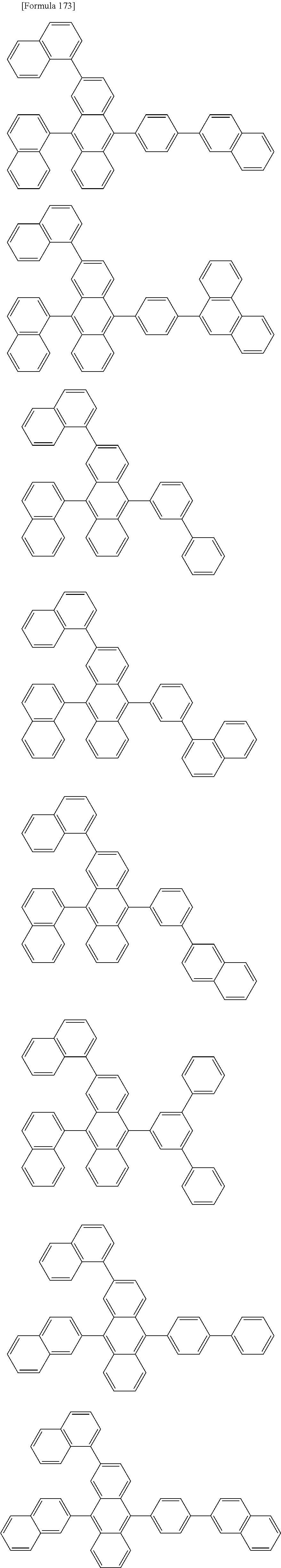

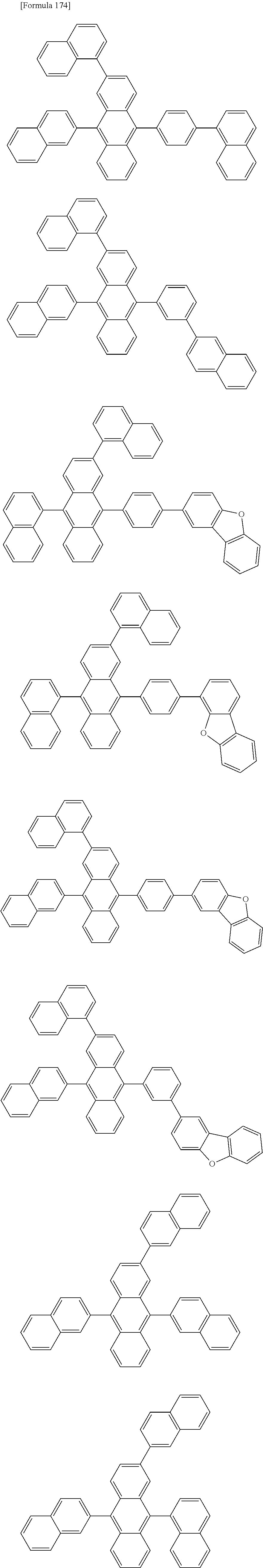

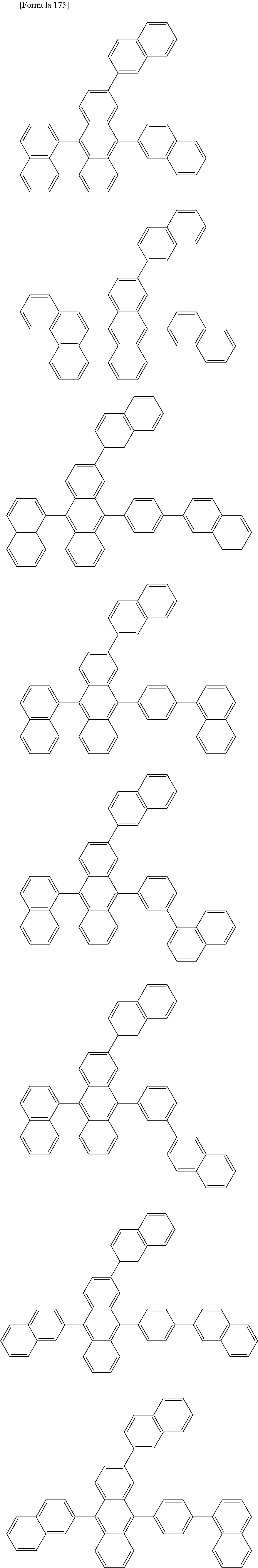

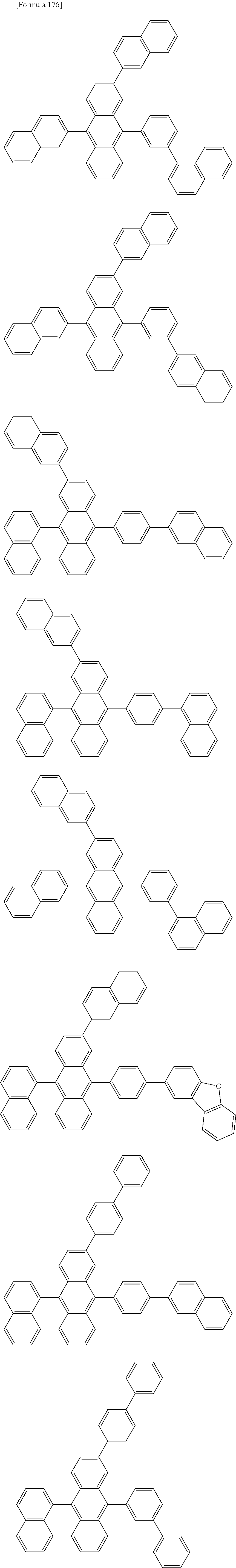

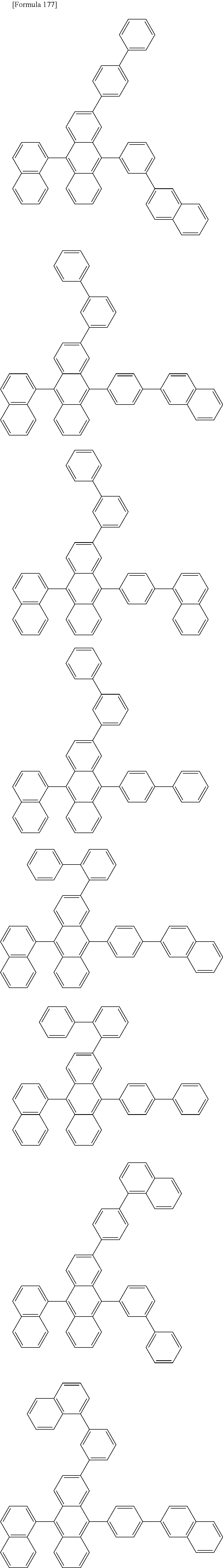

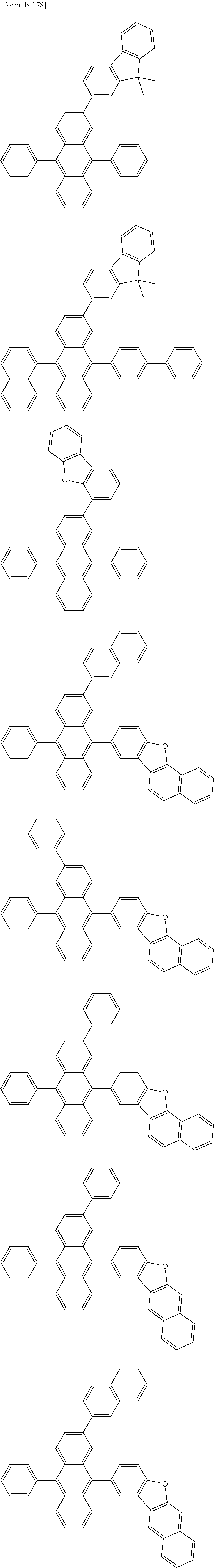

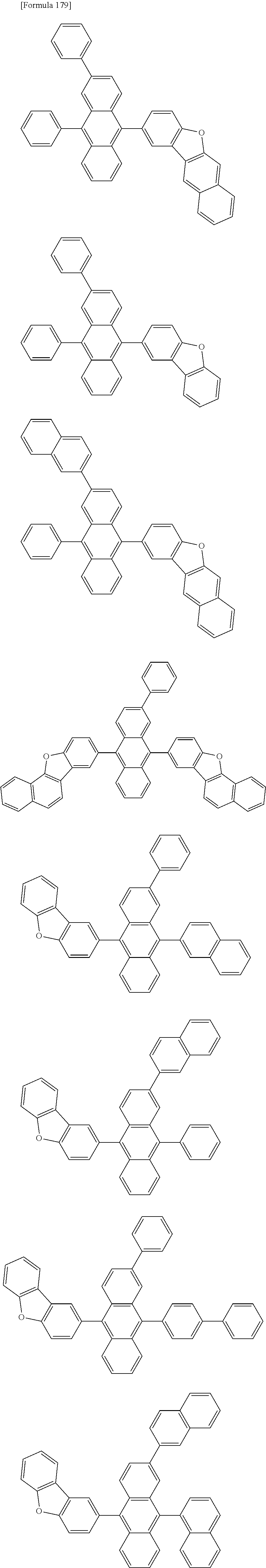

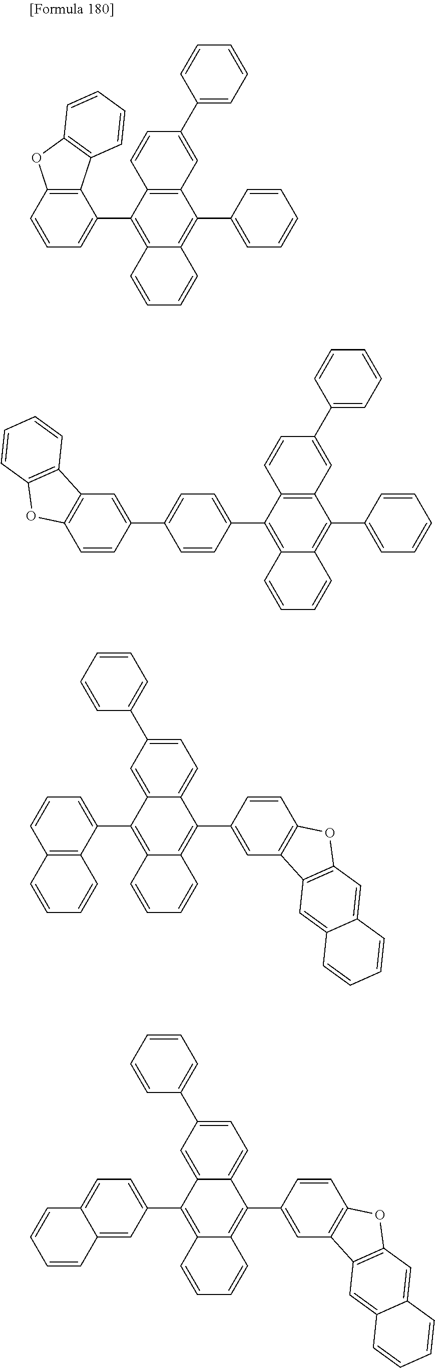 [SMILES: c1ccc(-c2ccc3c(-c4cccc5oc6ccccc6c45)c4ccccc4c(-c4ccccc4)c3c2)cc1 ; c1ccc(-c2ccc3c(-c4ccc(-c5ccc6oc7ccccc7c6c5)cc4)c4ccccc4c(-c4ccccc4)c3c2)cc1 ; c1ccc(-c2ccc3c(-c4ccc5oc6cc7ccccc7cc6c5c4)c4ccccc4c(-c4cccc5ccccc45)c3c2)cc1 ; c1ccc(-c2ccc3c(-c4ccc5oc6cc7ccccc7cc6c5c4)c4ccccc4c(-c4ccc5ccccc5c4)c3c2)cc1]

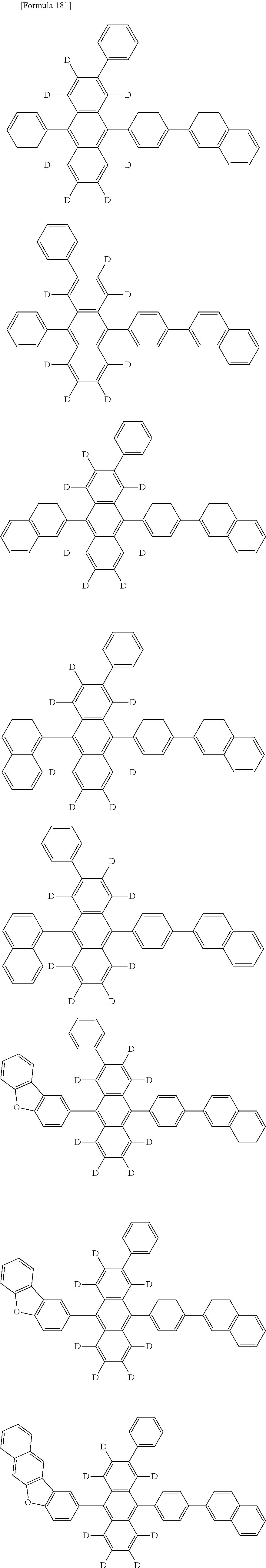

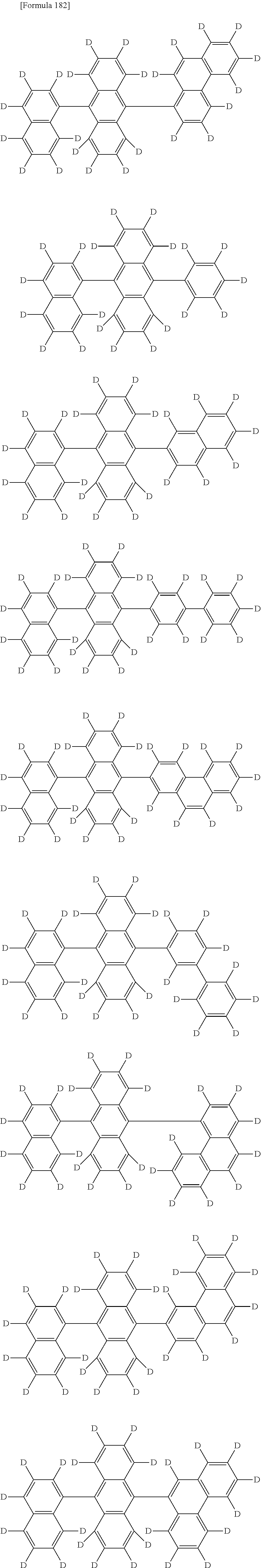

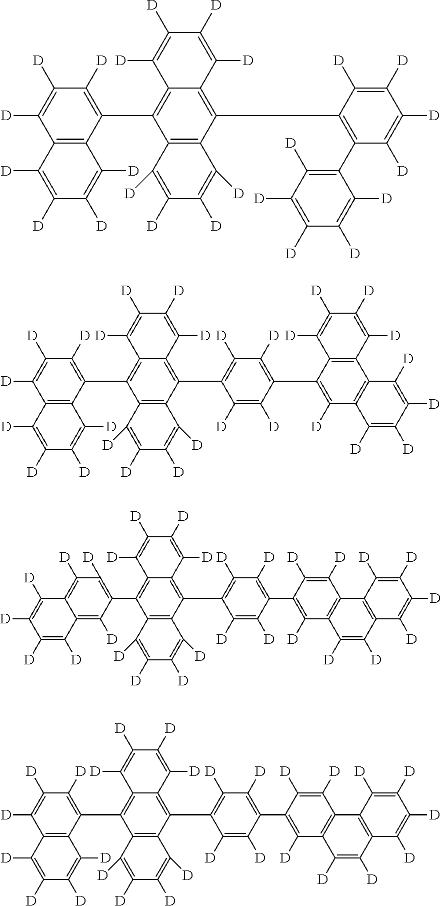

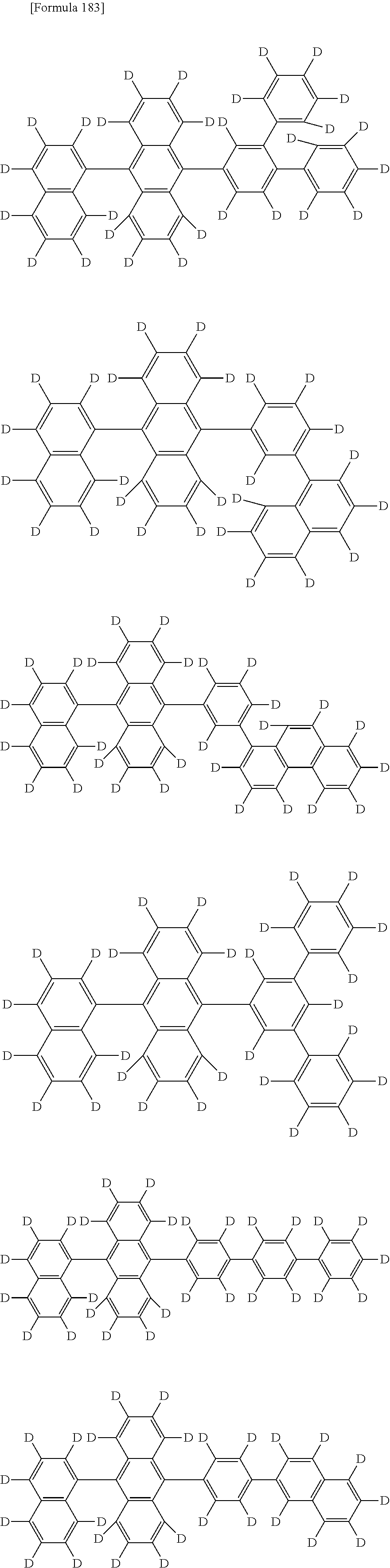

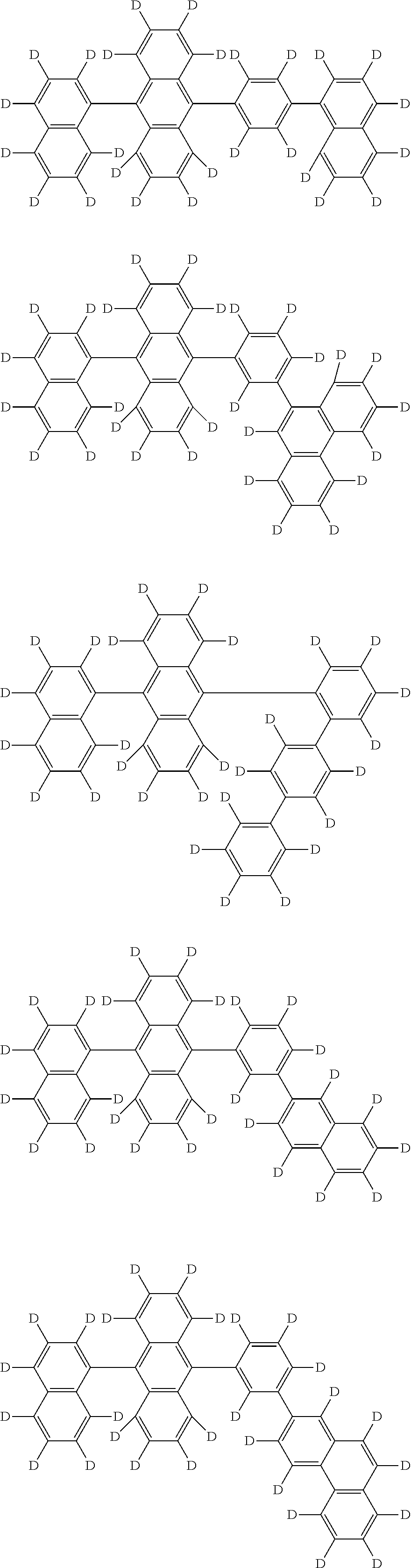

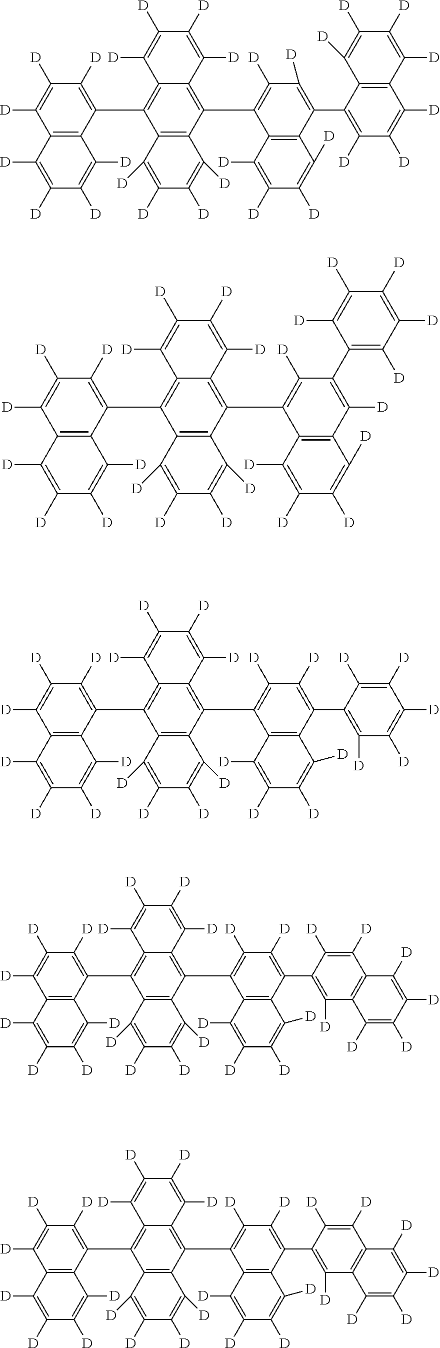

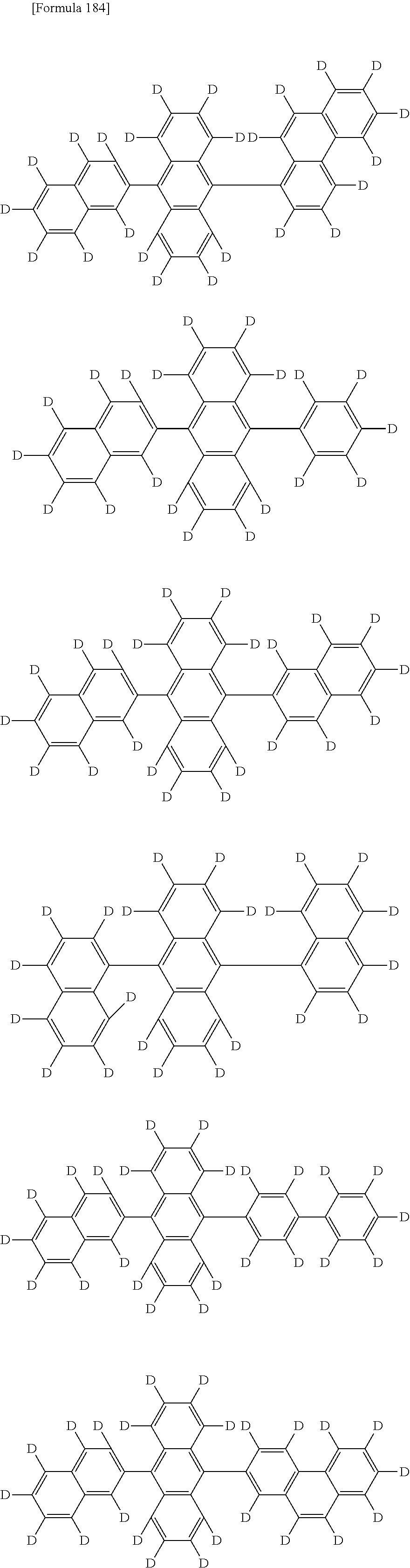

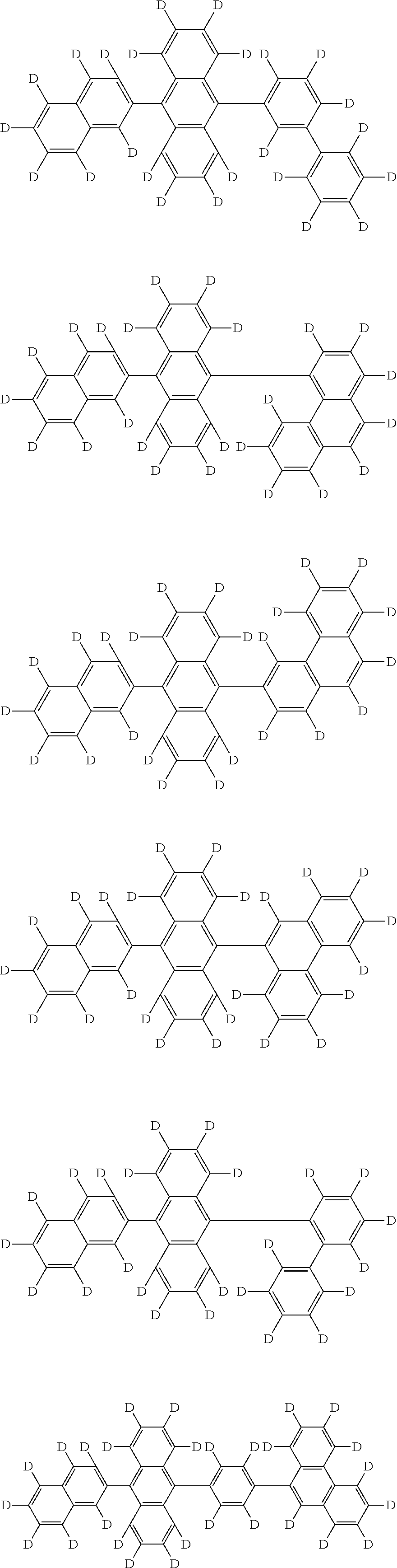

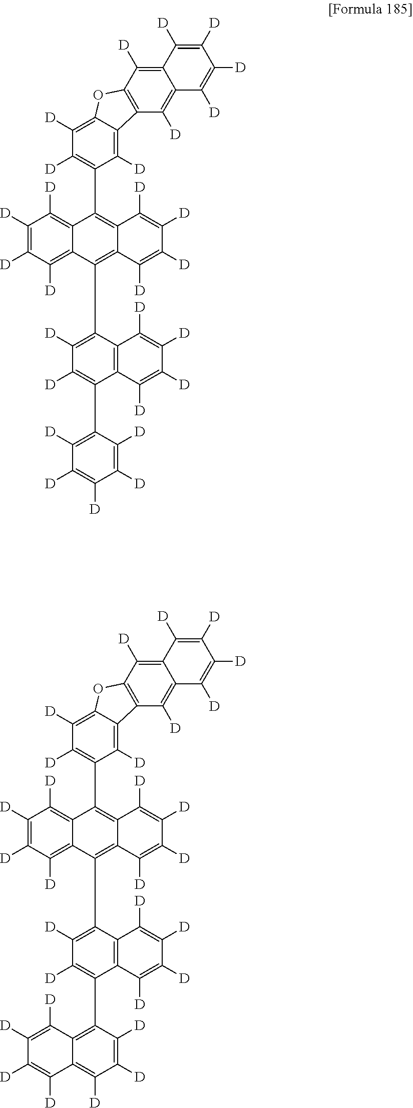

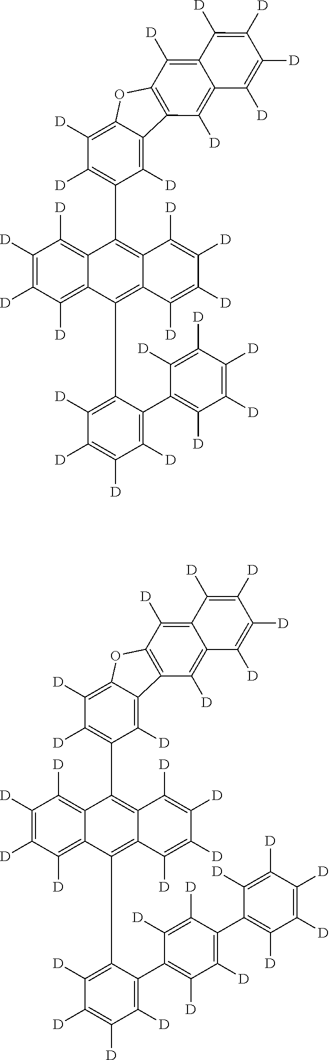 [SMILES: [2H]c1c([2H])c([2H])c(-c2c([2H])c([2H])c([2H])c([2H])c2-c2c3c([2H])c([2H])c([2H])c([2H])c3c(-c3c([2H])c([2H])c4oc5c([2H])c6c([2H])c([2H])c([2H])c([2H])c6c([2H])c5c4c3[2H])c3c([2H])c([2H])c([2H])c([2H])c23)c([2H])c1[2H] ; [2H]c1c([2H])c([2H])c(-c2c([2H])c([2H])c(-c3c([2H])c([2H])c([2H])c([2H])c3-c3c4c([2H])c([2H])c([2H])c([2H])c4c(-c4c([2H])c([2H])c5oc6c([2H])c7c([2H])c([2H])c([2H])c([2H])c7c([2H])c6c5c4[2H])c4c([2H])c([2H])c([2H])c([2H])c34)c([2H])c2[2H])c([2H])c1[2H]]

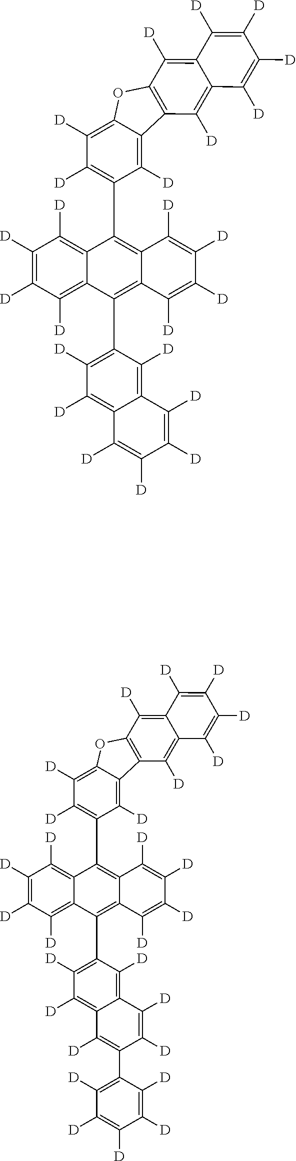 [SMILES: [2H]c1c(-c2c3c([2H])c([2H])c([2H])c([2H])c3c(-c3c([2H])c([2H])c4c([2H])c([2H])c([2H])c([2H])c4c3[2H])c3c([2H])c([2H])c([2H])c([2H])c23)c([2H])c2c(oc3c([2H])c4c([2H])c([2H])c([2H])c([2H])c4c([2H])c32)c1[2H] ; [2H]c1c([2H])c([2H])c(-c2c([2H])c([2H])c3c([2H])c(-c4c5c([2H])c([2H])c([2H])c([2H])c5c(-c5c([2H])c([2H])c6oc7c([2H])c8c([2H])c([2H])c([2H])c([2H])c8c([2H])c7c6c5[2H])c5c([2H])c([2H])c([2H])c([2H])c45)c([2H])c([2H])c3c2[2H])c([2H])c1[2H]]

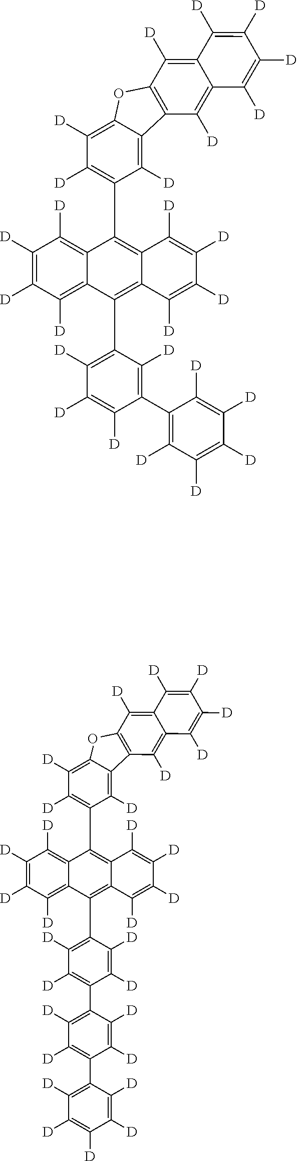 [SMILES: [2H]c1c([2H])c([2H])c(-c2c([2H])c([2H])c([2H])c(-c3c4c([2H])c([2H])c([2H])c([2H])c4c(-c4c([2H])c([2H])c5oc6c([2H])c7c([2H])c([2H])c([2H])c([2H])c7c([2H])c6c5c4[2H])c4c([2H])c([2H])c([2H])c([2H])c34)c2[2H])c([2H])c1[2H] ; [2H]c1c([2H])c([2H])c(-c2c([2H])c([2H])c(-c3c([2H])c([2H])c(-c4c5c([2H])c([2H])c([2H])c([2H])c5c(-c5c([2H])c([2H])c6oc7c([2H])c8c([2H])c([2H])c([2H])c([2H])c8c([2H])c7c6c5[2H])c5c([2H])c([2H])c([2H])c([2H])c45)c([2H])c3[2H])c([2H])c2[2H])c([2H])c1[2H]]

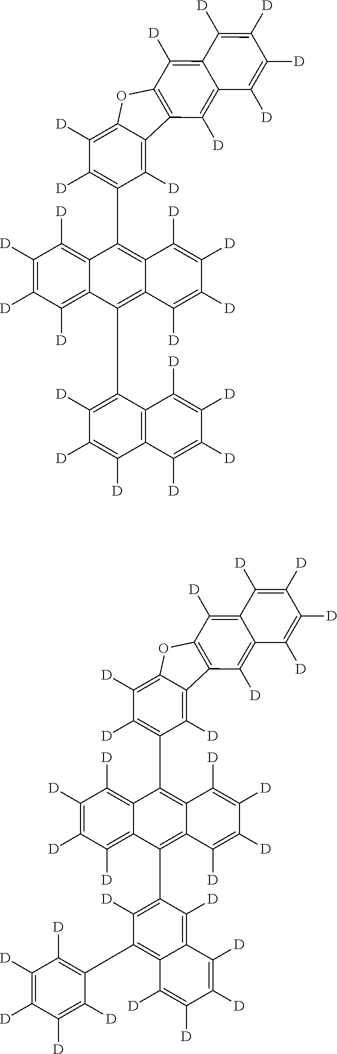 [SMILES: [2H]c1c(-c2c3c([2H])c([2H])c([2H])c([2H])c3c(-c3c([2H])c([2H])c([2H])c4c([2H])c([2H])c([2H])c([2H])c34)c3c([2H])c([2H])c([2H])c([2H])c23)c([2H])c2c(oc3c([2H])c4c([2H])c([2H])c([2H])c([2H])c4c([2H])c32)c1[2H] ; [2H]c1c([2H])c([2H])c(-c2c([2H])c(-c3c4c([2H])c([2H])c([2H])c([2H])c4c(-c4c([2H])c([2H])c5oc6c([2H])c7c([2H])c([2H])c([2H])c([2H])c7c([2H])c6c5c4[2H])c4c([2H])c([2H])c([2H])c([2H])c34)c([2H])c3c([2H])c([2H])c([2H])c([2H])c23)c([2H])c1[2H]]

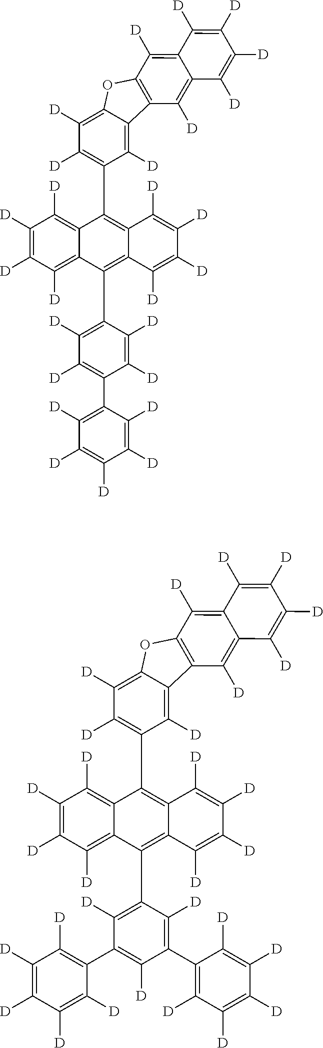 [SMILES: [2H]c1c([2H])c([2H])c(-c2c([2H])c([2H])c(-c3c4c([2H])c([2H])c([2H])c([2H])c4c(-c4c([2H])c([2H])c5oc6c([2H])c7c([2H])c([2H])c([2H])c([2H])c7c([2H])c6c5c4[2H])c4c([2H])c([2H])c([2H])c([2H])c34)c([2H])c2[2H])c([2H])c1[2H] ; [2H]c1c([2H])c([2H])c(-c2c([2H])c(-c3c([2H])c([2H])c([2H])c([2H])c3[2H])c([2H])c(-c3c4c([2H])c([2H])c([2H])c([2H])c4c(-c4c([2H])c([2H])c5oc6c([2H])c7c([2H])c([2H])c([2H])c([2H])c7c([2H])c6c5c4[2H])c4c([2H])c([2H])c([2H])c([2H])c34)c2[2H])c([2H])c1[2H]]

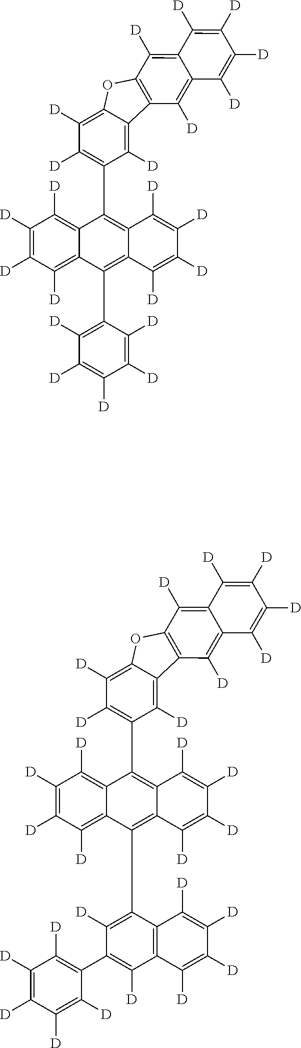 [SMILES: [2H]c1c([2H])c([2H])c(-c2c3c([2H])c([2H])c([2H])c([2H])c3c(-c3c([2H])c([2H])c4oc5c([2H])c6c([2H])c([2H])c([2H])c([2H])c6c([2H])c5c4c3[2H])c3c([2H])c([2H])c([2H])c([2H])c23)c([2H])c1[2H] ; [2H]c1c([2H])c([2H])c(-c2c([2H])c(-c3c4c([2H])c([2H])c([2H])c([2H])c4c(-c4c([2H])c([2H])c5oc6c([2H])c7c([2H])c([2H])c([2H])c([2H])c7c([2H])c6c5c4[2H])c4c([2H])c([2H])c([2H])c([2H])c34)c3c([2H])c([2H])c([2H])c([2H])c3c2[2H])c([2H])c1[2H]]

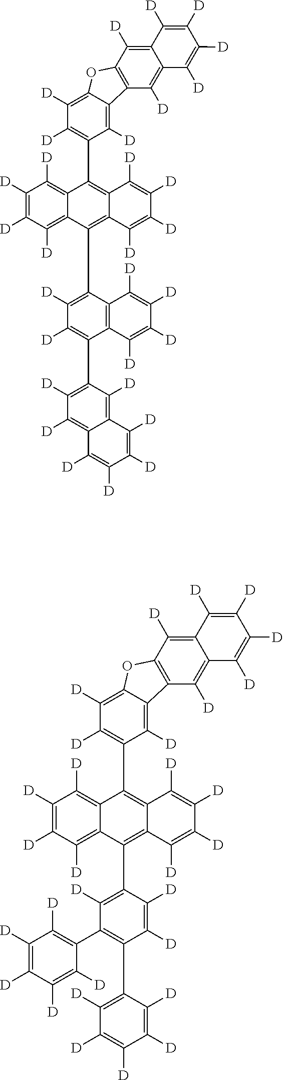

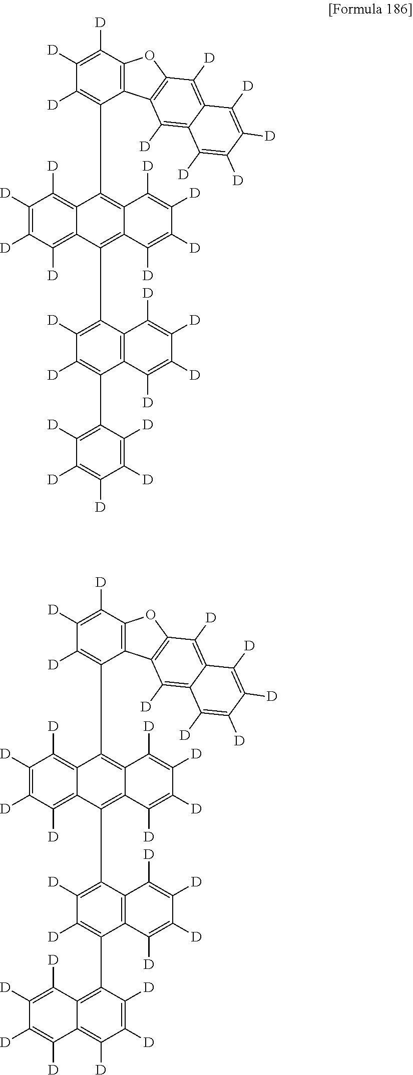

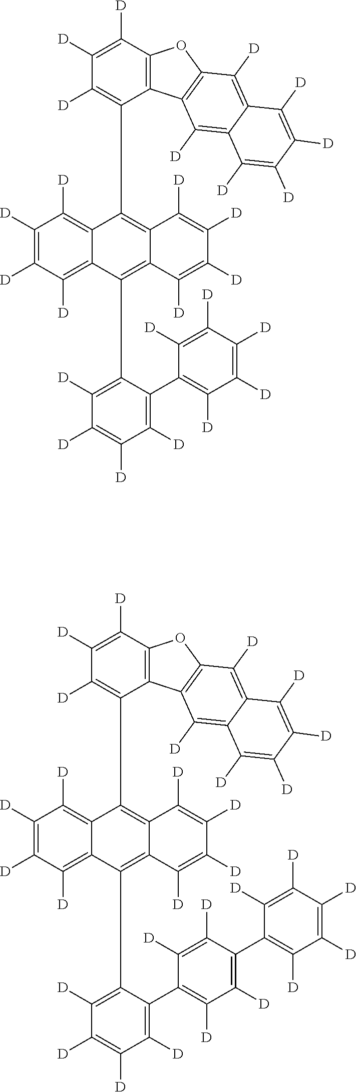 [SMILES: [2H]c1c([2H])c([2H])c(-c2c([2H])c([2H])c([2H])c([2H])c2-c2c3c([2H])c([2H])c([2H])c([2H])c3c(-c3c([2H])c([2H])c([2H])c4oc5c([2H])c6c([2H])c([2H])c([2H])c([2H])c6c([2H])c5c34)c3c([2H])c([2H])c([2H])c([2H])c23)c([2H])c1[2H] ; [2H]c1c([2H])c([2H])c(-c2c([2H])c([2H])c(-c3c([2H])c([2H])c([2H])c([2H])c3-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c([2H])c2[2H])c([2H])c1[2H]]

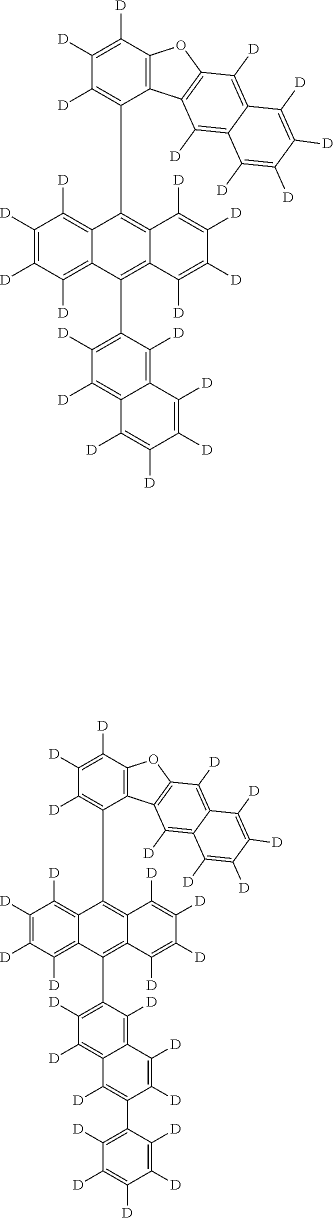 [SMILES: [2H]c1c([2H])c(-c2c3c([2H])c([2H])c([2H])c([2H])c3c(-c3c([2H])c([2H])c4c([2H])c([2H])c([2H])c([2H])c4c3[2H])c3c([2H])c([2H])c([2H])c([2H])c23)c2c(oc3c([2H])c4c([2H])c([2H])c([2H])c([2H])c4c([2H])c32)c1[2H] ; [2H]c1c([2H])c([2H])c(-c2c([2H])c([2H])c3c([2H])c(-c4c5c([2H])c([2H])c([2H])c([2H])c5c(-c5c([2H])c([2H])c([2H])c6oc7c([2H])c8c([2H])c([2H])c([2H])c([2H])c8c([2H])c7c56)c5c([2H])c([2H])c([2H])c([2H])c45)c([2H])c([2H])c3c2[2H])c([2H])c1[2H]]

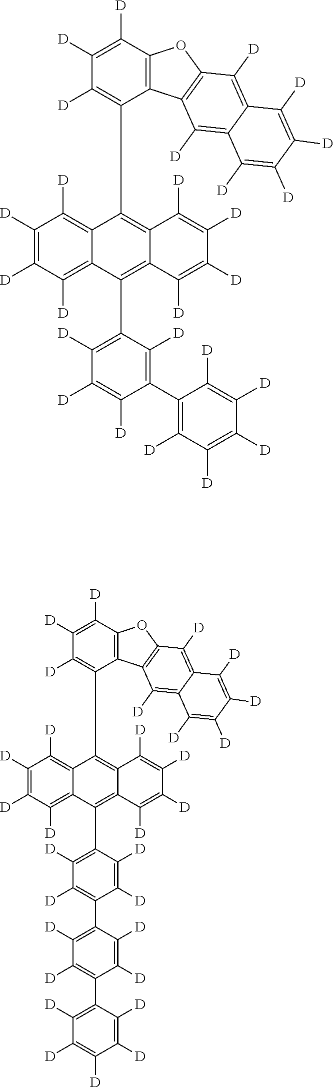 [SMILES: [2H]c1c([2H])c([2H])c(-c2c([2H])c([2H])c([2H])c(-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c2[2H])c([2H])c1[2H] ; [2H]c1c([2H])c([2H])c(-c2c([2H])c([2H])c(-c3c([2H])c([2H])c(-c4c5c([2H])c([2H])c([2H])c([2H])c5c(-c5c([2H])c([2H])c([2H])c6oc7c([2H])c8c([2H])c([2H])c([2H])c([2H])c8c([2H])c7c56)c5c([2H])c([2H])c([2H])c([2H])c45)c([2H])c3[2H])c([2H])c2[2H])c([2H])c1[2H]]

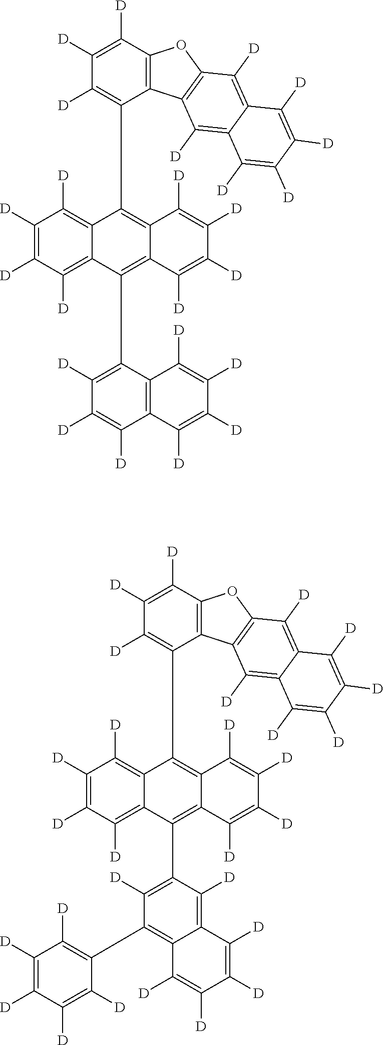 [SMILES: [2H]c1c([2H])c(-c2c3c([2H])c([2H])c([2H])c([2H])c3c(-c3c([2H])c([2H])c([2H])c4c([2H])c([2H])c([2H])c([2H])c34)c3c([2H])c([2H])c([2H])c([2H])c23)c2c(oc3c([2H])c4c([2H])c([2H])c([2H])c([2H])c4c([2H])c32)c1[2H] ; [2H]c1c([2H])c([2H])c(-c2c([2H])c(-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c([2H])c3c([2H])c([2H])c([2H])c([2H])c23)c([2H])c1[2H]]

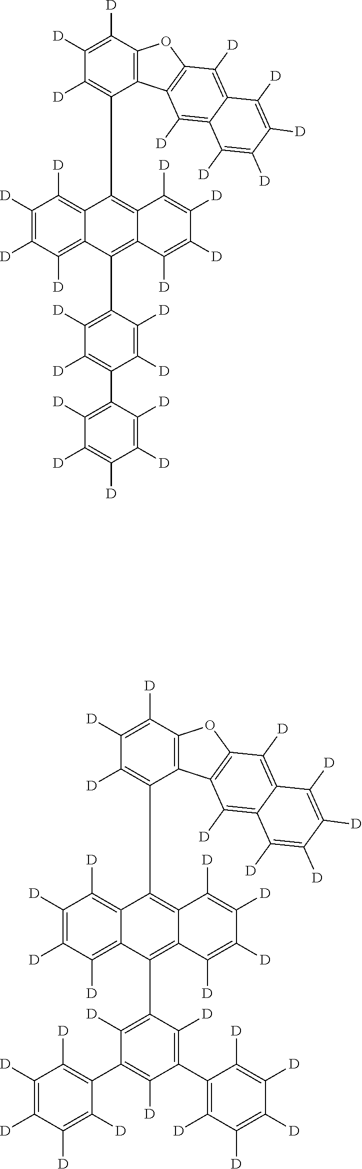 [SMILES: [2H]c1c([2H])c([2H])c(-c2c([2H])c([2H])c(-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c([2H])c2[2H])c([2H])c1[2H] ; [2H]c1c([2H])c([2H])c(-c2c([2H])c(-c3c([2H])c([2H])c([2H])c([2H])c3[2H])c([2H])c(-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c2[2H])c([2H])c1[2H]]

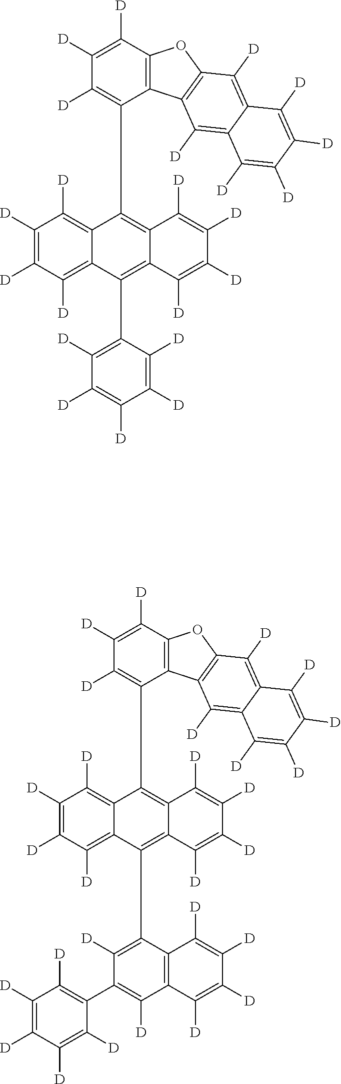 [SMILES: [2H]c1c([2H])c([2H])c(-c2c3c([2H])c([2H])c([2H])c([2H])c3c(-c3c([2H])c([2H])c([2H])c4oc5c([2H])c6c([2H])c([2H])c([2H])c([2H])c6c([2H])c5c34)c3c([2H])c([2H])c([2H])c([2H])c23)c([2H])c1[2H] ; [2H]c1c([2H])c([2H])c(-c2c([2H])c(-c3c4c([2H])c([2H])c([2H])c([2H])c4c(-c4c([2H])c([2H])c([2H])c5oc6c([2H])c7c([2H])c([2H])c([2H])c([2H])c7c([2H])c6c45)c4c([2H])c([2H])c([2H])c([2H])c34)c3c([2H])c([2H])c([2H])c([2H])c3c2[2H])c([2H])c1[2H]]

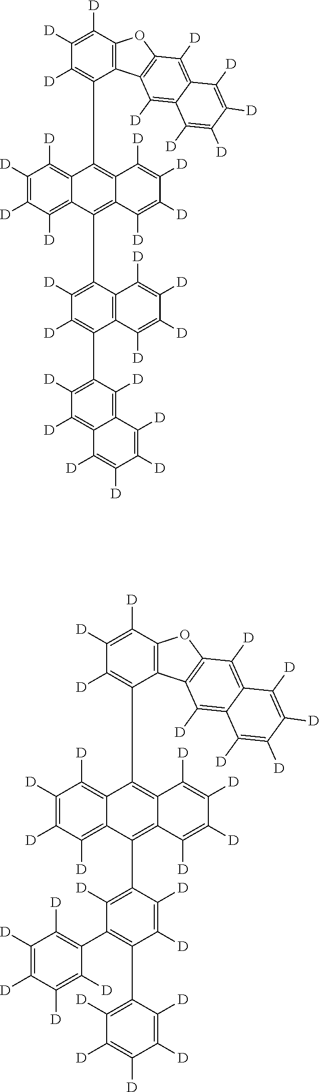

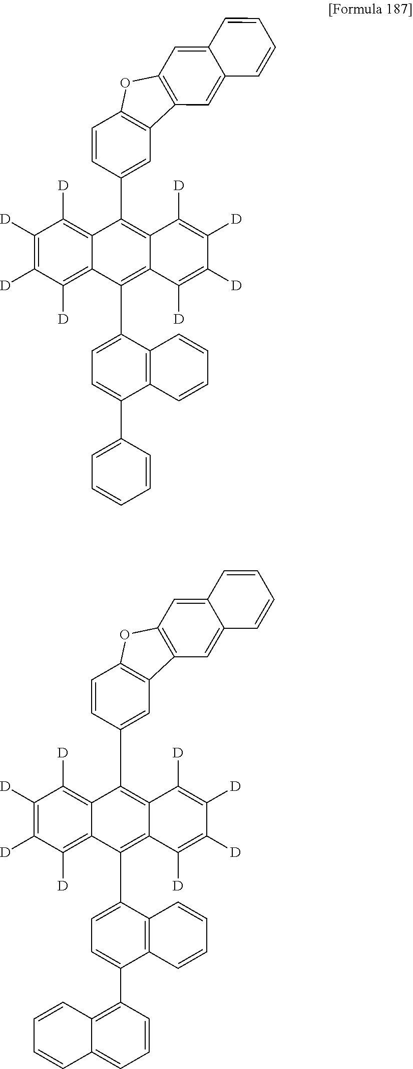 [SMILES: [2H]c1c([2H])c([2H])c2c(-c3ccc(-c4ccccc4)c4ccccc34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc(-c4cccc5ccccc45)c4ccccc34)c3c([2H])c([2H])c([2H])c([2H])c3c(-c3ccc4oc5cc6ccccc6cc5c4c3)c2c1[2H]]

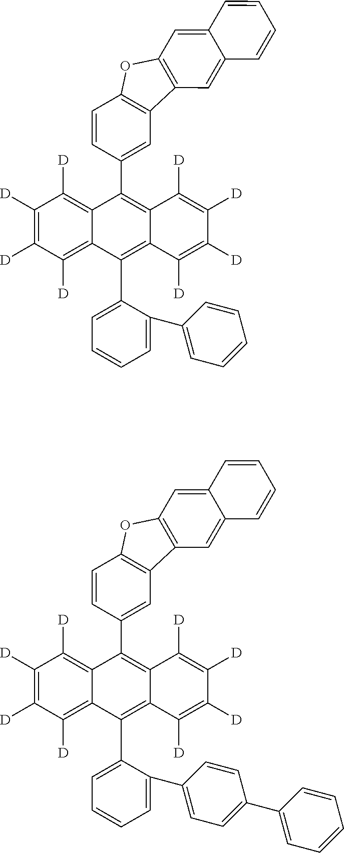 [SMILES: [2H]c1c([2H])c([2H])c2c(-c3ccccc3-c3ccccc3)c3c([2H])c([2H])c([2H])c([2H])c3c(-c3ccc4oc5cc6ccccc6cc5c4c3)c2c1[2H] ; [2H]c1c([2H])c([2H])c2c(-c3ccccc3-c3ccc(-c4ccccc4)cc3)c3c([2H])c([2H])c([2H])c([2H])c3c(-c3ccc4oc5cc6ccccc6cc5c4c3)c2c1[2H]]

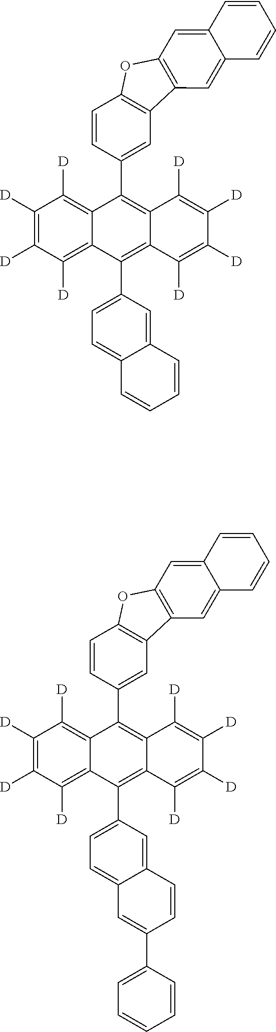 [SMILES: [2H]c1c([2H])c([2H])c2c(-c3ccc4oc5cc6ccccc6cc5c4c3)c3c([2H])c([2H])c([2H])c([2H])c3c(-c3ccc4ccccc4c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc4cc(-c5ccccc5)ccc4c3)c2c1[2H]]

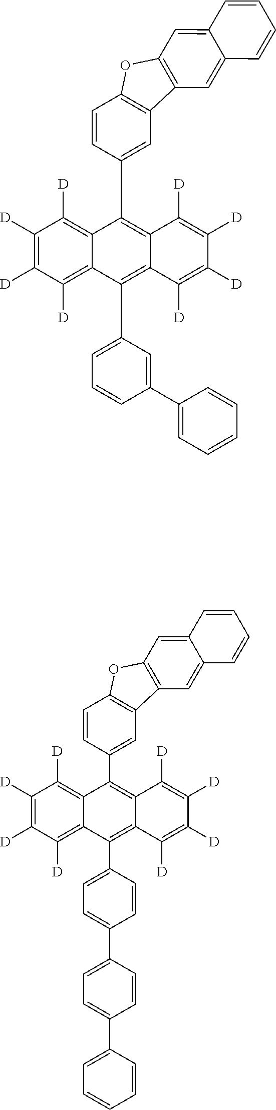 [SMILES: [2H]c1c([2H])c([2H])c2c(-c3ccc4oc5cc6ccccc6cc5c4c3)c3c([2H])c([2H])c([2H])c([2H])c3c(-c3cccc(-c4ccccc4)c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc(-c4ccc(-c5ccccc5)cc4)cc3)c2c1[2H]]

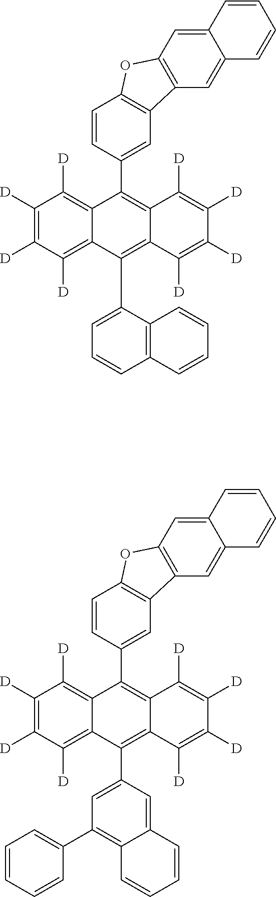 [SMILES: [2H]c1c([2H])c([2H])c2c(-c3cccc4ccccc34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc4oc5cc6ccccc6cc5c4c3)c3c([2H])c([2H])c([2H])c([2H])c3c(-c3cc(-c4ccccc4)c4ccccc4c3)c2c1[2H]]

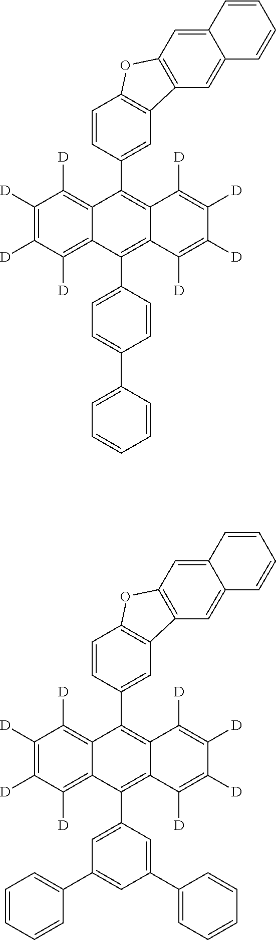 [SMILES: [2H]c1c([2H])c([2H])c2c(-c3ccc4oc5cc6ccccc6cc5c4c3)c3c([2H])c([2H])c([2H])c([2H])c3c(-c3ccc(-c4ccccc4)cc3)c2c1[2H] ; [2H]c1c([2H])c([2H])c2c(-c3ccc4oc5cc6ccccc6cc5c4c3)c3c([2H])c([2H])c([2H])c([2H])c3c(-c3cc(-c4ccccc4)cc(-c4ccccc4)c3)c2c1[2H]]

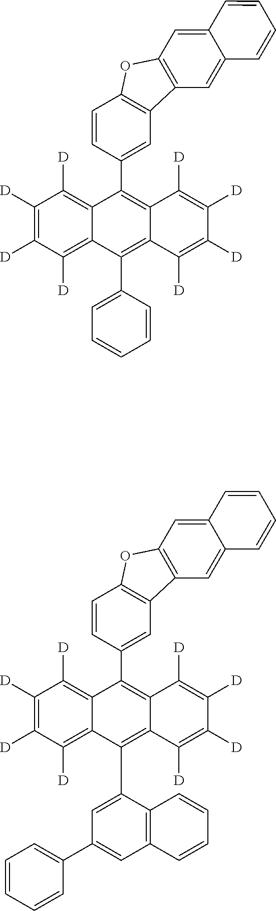 [SMILES: [2H]c1c([2H])c([2H])c2c(-c3ccc4oc5cc6ccccc6cc5c4c3)c3c([2H])c([2H])c([2H])c([2H])c3c(-c3ccccc3)c2c1[2H] ; [2H]c1c([2H])c([2H])c2c(-c3cc(-c4ccccc4)cc4ccccc34)c3c([2H])c([2H])c([2H])c([2H])c3c(-c3ccc4oc5cc6ccccc6cc5c4c3)c2c1[2H]]

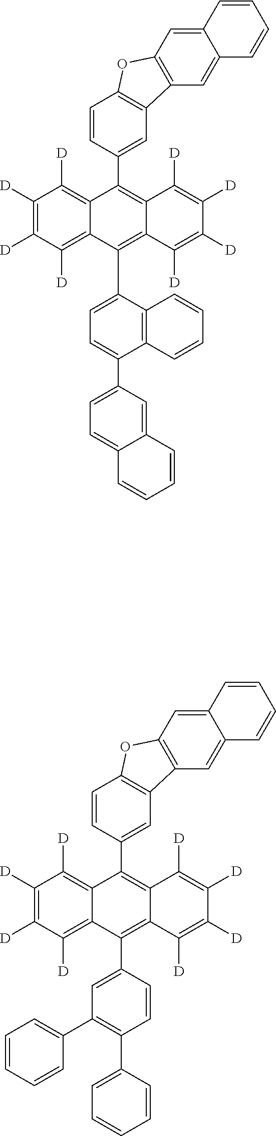 [SMILES: [2H]c1c([2H])c([2H])c2c(-c3ccc(-c4ccc5ccccc5c4)c4ccccc34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc(-c4ccccc4)c(-c4ccccc4)c3)c2c1[2H]]

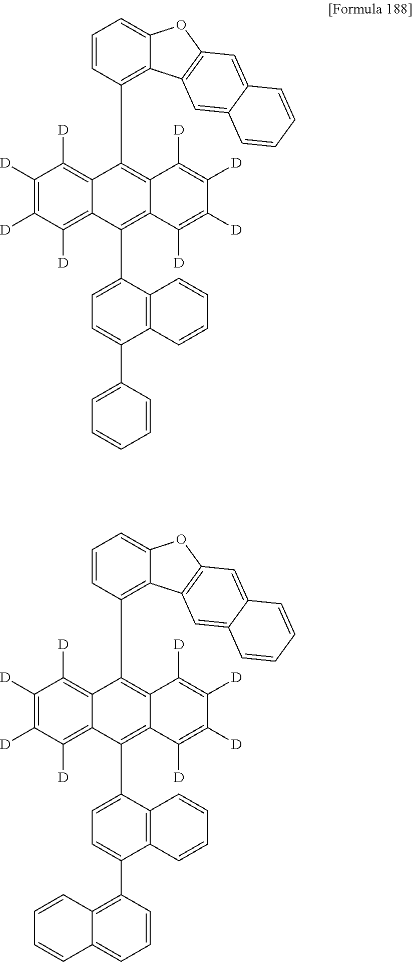 [SMILES: [2H]c1c([2H])c([2H])c2c(-c3cccc4oc5cc6ccccc6cc5c34)c3c([2H])c([2H])c([2H])c([2H])c3c(-c3ccc(-c4ccccc4)c4ccccc34)c2c1[2H] ; [2H]c1c([2H])c([2H])c2c(-c3cccc4oc5cc6ccccc6cc5c34)c3c([2H])c([2H])c([2H])c([2H])c3c(-c3ccc(-c4cccc5ccccc45)c4ccccc34)c2c1[2H]]

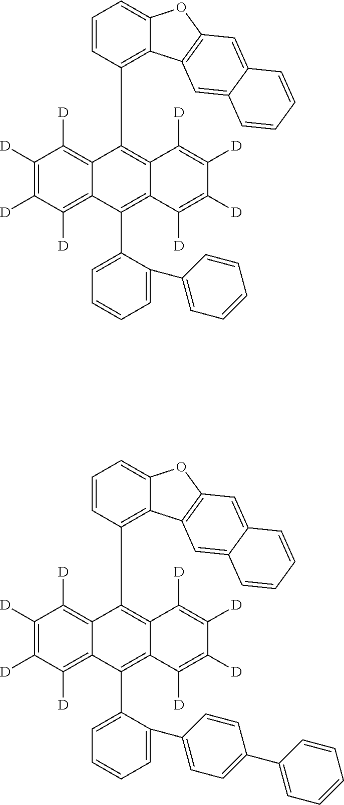 [SMILES: [2H]c1c([2H])c([2H])c2c(-c3cccc4oc5cc6ccccc6cc5c34)c3c([2H])c([2H])c([2H])c([2H])c3c(-c3ccccc3-c3ccccc3)c2c1[2H] ; [2H]c1c([2H])c([2H])c2c(-c3cccc4oc5cc6ccccc6cc5c34)c3c([2H])c([2H])c([2H])c([2H])c3c(-c3ccccc3-c3ccc(-c4ccccc4)cc3)c2c1[2H]]

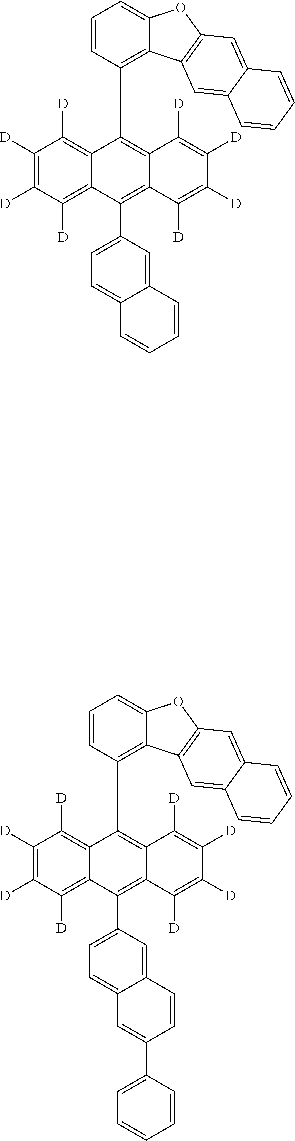 [SMILES: [2H]c1c([2H])c([2H])c2c(-c3cccc4oc5cc6ccccc6cc5c34)c3c([2H])c([2H])c([2H])c([2H])c3c(-c3ccc4ccccc4c3)c2c1[2H] ; [2H]c1c([2H])c([2H])c2c(-c3cccc4oc5cc6ccccc6cc5c34)c3c([2H])c([2H])c([2H])c([2H])c3c(-c3ccc4cc(-c5ccccc5)ccc4c3)c2c1[2H]]

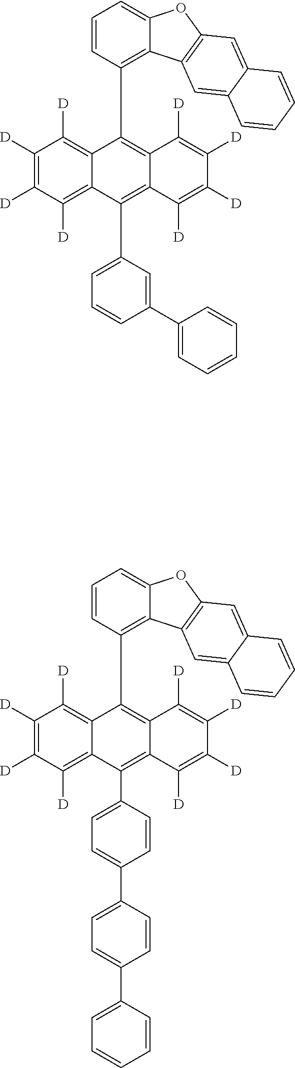 [SMILES: [2H]c1c([2H])c([2H])c2c(-c3cccc4oc5cc6ccccc6cc5c34)c3c([2H])c([2H])c([2H])c([2H])c3c(-c3cccc(-c4ccccc4)c3)c2c1[2H] ; [2H]c1c([2H])c([2H])c2c(-c3cccc4oc5cc6ccccc6cc5c34)c3c([2H])c([2H])c([2H])c([2H])c3c(-c3ccc(-c4ccc(-c5ccccc5)cc4)cc3)c2c1[2H]]

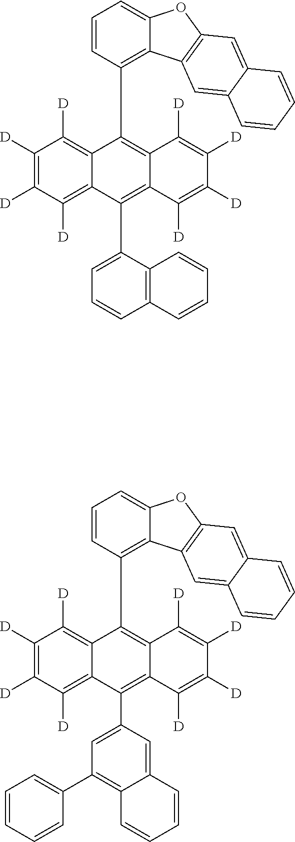 [SMILES: [2H]c1c([2H])c([2H])c2c(-c3cccc4oc5cc6ccccc6cc5c34)c3c([2H])c([2H])c([2H])c([2H])c3c(-c3cccc4ccccc34)c2c1[2H] ; [2H]c1c([2H])c([2H])c2c(-c3cccc4oc5cc6ccccc6cc5c34)c3c([2H])c([2H])c([2H])c([2H])c3c(-c3cc(-c4ccccc4)c4ccccc4c3)c2c1[2H]]

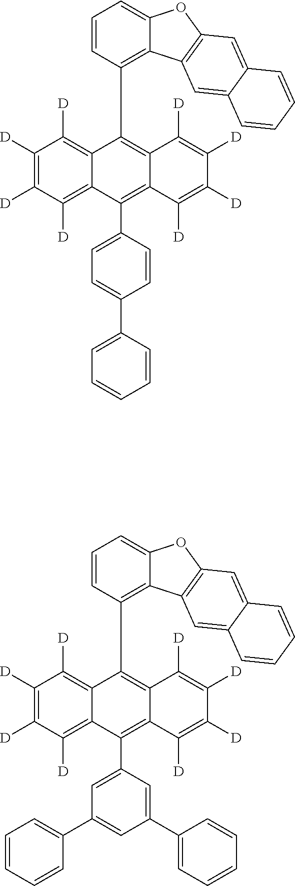 [SMILES: [2H]c1c([2H])c([2H])c2c(-c3cccc4oc5cc6ccccc6cc5c34)c3c([2H])c([2H])c([2H])c([2H])c3c(-c3ccc(-c4ccccc4)cc3)c2c1[2H] ; [2H]c1c([2H])c([2H])c2c(-c3cccc4oc5cc6ccccc6cc5c34)c3c([2H])c([2H])c([2H])c([2H])c3c(-c3cc(-c4ccccc4)cc(-c4ccccc4)c3)c2c1[2H]]

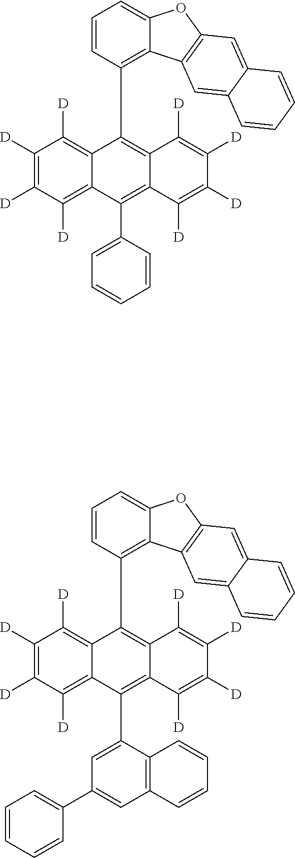 [SMILES: [2H]c1c([2H])c([2H])c2c(-c3cccc4oc5cc6ccccc6cc5c34)c3c([2H])c([2H])c([2H])c([2H])c3c(-c3ccccc3)c2c1[2H] ; [2H]c1c([2H])c([2H])c2c(-c3cccc4oc5cc6ccccc6cc5c34)c3c([2H])c([2H])c([2H])c([2H])c3c(-c3cc(-c4ccccc4)cc4ccccc34)c2c1[2H]]

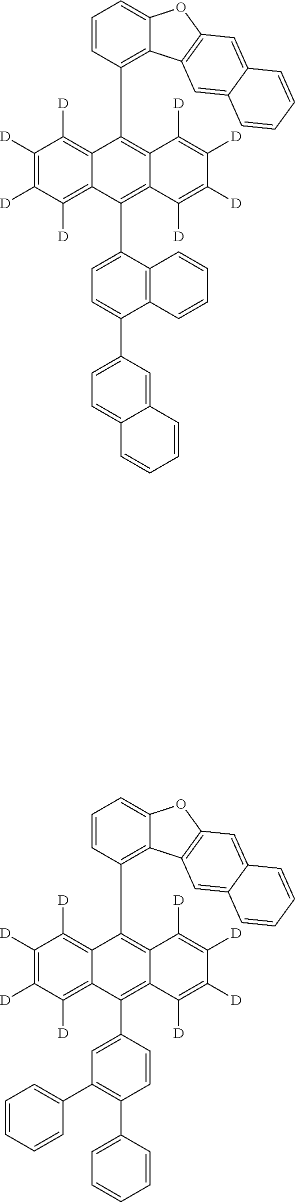 [SMILES: [2H]c1c([2H])c([2H])c2c(-c3cccc4oc5cc6ccccc6cc5c34)c3c([2H])c([2H])c([2H])c([2H])c3c(-c3ccc(-c4ccc5ccccc5c4)c4ccccc34)c2c1[2H] ; [2H]c1c([2H])c([2H])c2c(-c3cccc4oc5cc6ccccc6cc5c34)c3c([2H])c([2H])c([2H])c([2H])c3c(-c3ccc(-c4ccccc4)c(-c4ccccc4)c3)c2c1[2H]]

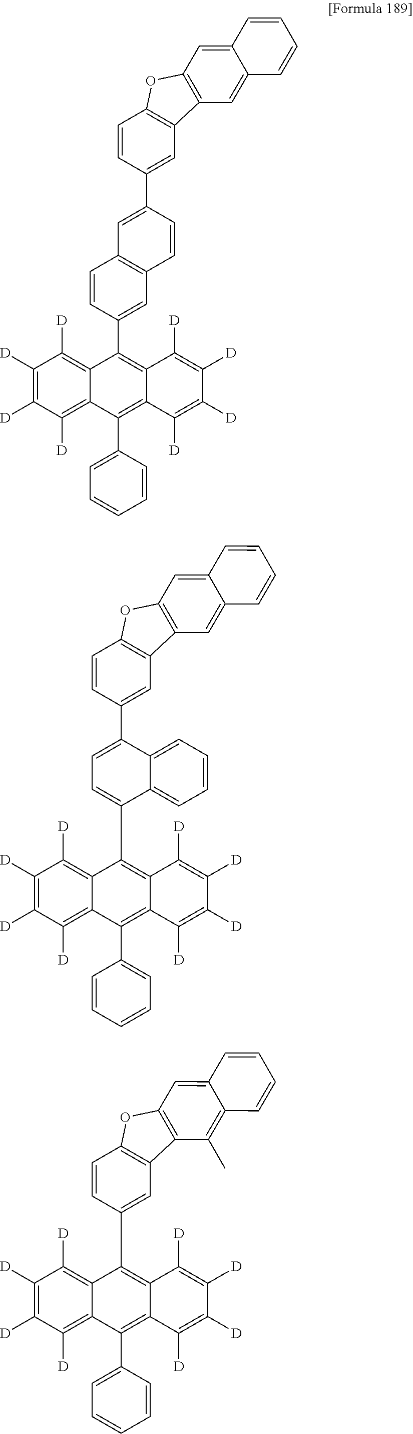 [SMILES: [2H]c1c([2H])c([2H])c2c(-c3ccc4cc(-c5ccc6oc7cc8ccccc8cc7c6c5)ccc4c3)c3c([2H])c([2H])c([2H])c([2H])c3c(-c3ccccc3)c2c1[2H] ; [2H]c1c([2H])c([2H])c2c(-c3ccc(-c4ccc5oc6cc7ccccc7cc6c5c4)c4ccccc34)c3c([2H])c([2H])c([2H])c([2H])c3c(-c3ccccc3)c2c1[2H] ; [2H]c1c([2H])c([2H])c2c(-c3ccc4oc5cc6ccccc6c(C)c5c4c3)c3c([2H])c([2H])c([2H])c([2H])c3c(-c3ccccc3)c2c1[2H]]

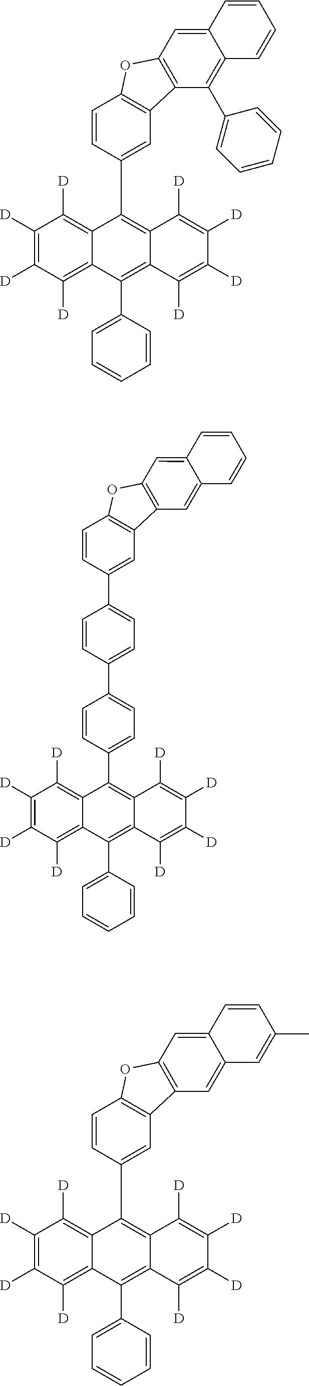 [SMILES: [2H]c1c([2H])c([2H])c2c(-c3ccc4oc5cc6ccccc6c(-c6ccccc6)c5c4c3)c3c([2H])c([2H])c([2H])c([2H])c3c(-c3ccccc3)c2c1[2H] ; [2H]c1c([2H])c([2H])c2c(-c3ccc(-c4ccc(-c5ccc6oc7cc8ccccc8cc7c6c5)cc4)cc3)c3c([2H])c([2H])c([2H])c([2H])c3c(-c3ccccc3)c2c1[2H] ; [2H]c1c([2H])c([2H])c2c(-c3ccc4oc5cc6ccc(C)cc6cc5c4c3)c3c([2H])c([2H])c([2H])c([2H])c3c(-c3ccccc3)c2c1[2H]]

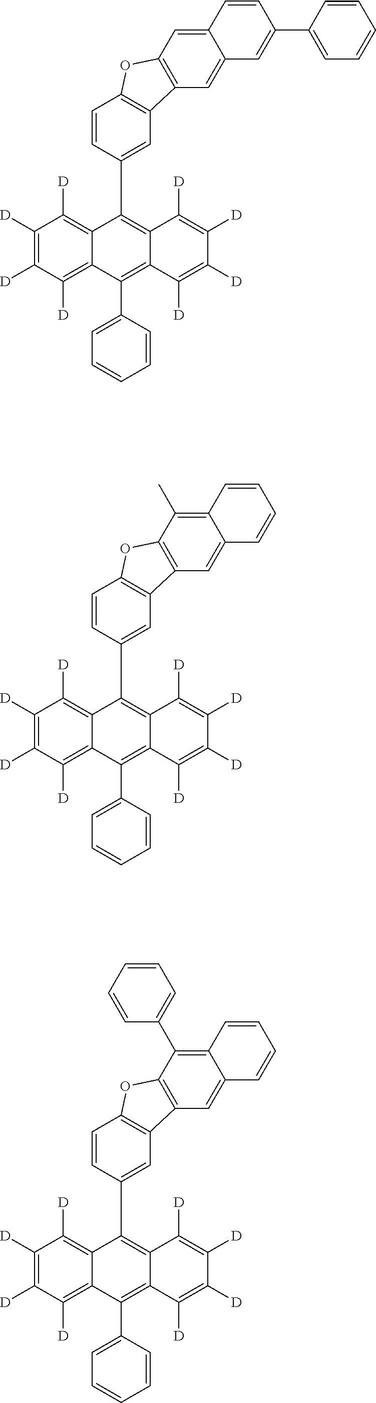 [SMILES: [2H]c1c([2H])c([2H])c2c(-c3ccc4oc5cc6ccc(-c7ccccc7)cc6cc5c4c3)c3c([2H])c([2H])c([2H])c([2H])c3c(-c3ccccc3)c2c1[2H] ; [2H]c1c([2H])c([2H])c2c(-c3ccc4oc5c(C)c6ccccc6cc5c4c3)c3c([2H])c([2H])c([2H])c([2H])c3c(-c3ccccc3)c2c1[2H] ; [2H]c1c([2H])c([2H])c2c(-c3ccc4oc5c(-c6ccccc6)c6ccccc6cc5c4c3)c3c([2H])c([2H])c([2H])c([2H])c3c(-c3ccccc3)c2c1[2H]]

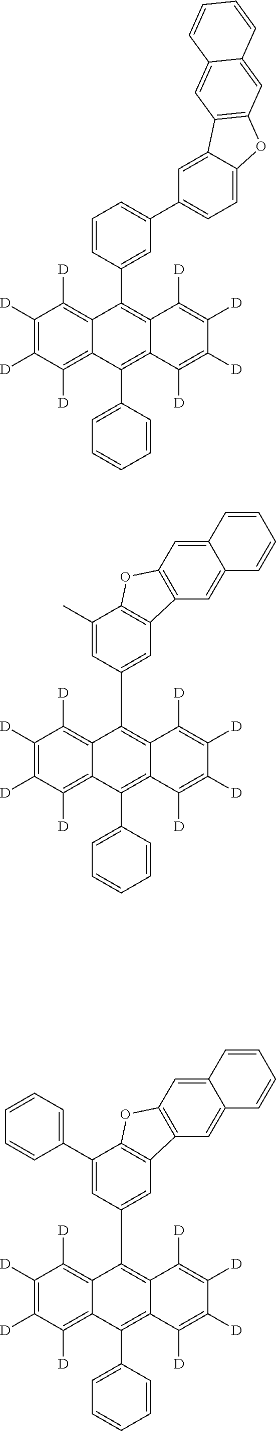 [SMILES: [2H]c1c([2H])c([2H])c2c(-c3cccc(-c4ccc5oc6cc7ccccc7cc6c5c4)c3)c3c([2H])c([2H])c([2H])c([2H])c3c(-c3ccccc3)c2c1[2H] ; [2H]c1c([2H])c([2H])c2c(-c3cc(C)c4oc5cc6ccccc6cc5c4c3)c3c([2H])c([2H])c([2H])c([2H])c3c(-c3ccccc3)c2c1[2H] ; [2H]c1c([2H])c([2H])c2c(-c3cc(-c4ccccc4)c4oc5cc6ccccc6cc5c4c3)c3c([2H])c([2H])c([2H])c([2H])c3c(-c3ccccc3)c2c1[2H]]

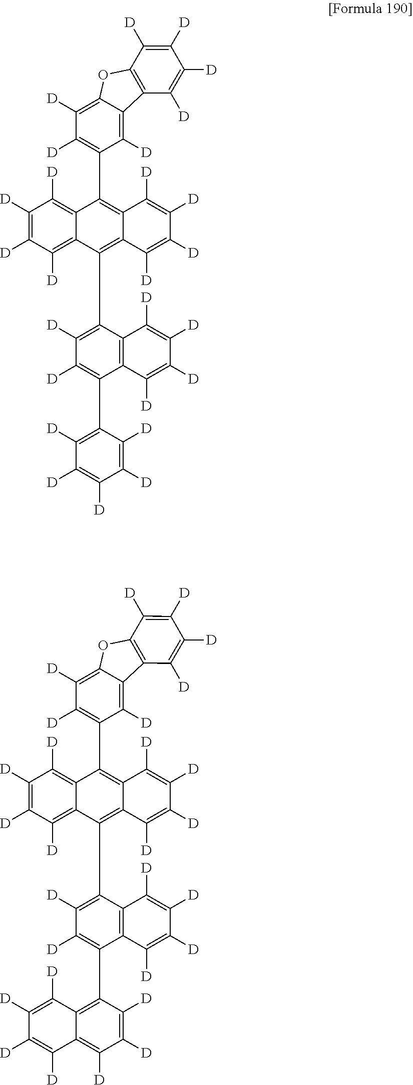 [SMILES: [2H]c1c([2H])c([2H])c(-c2c([2H])c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c3c([2H])c([2H])c([2H])c([2H])c23)c([2H])c1[2H] ; [2H]c1c([2H])c([2H])c2c(oc3c([2H])c([2H])c(-c4c5c([2H])c([2H])c([2H])c([2H])c5c(-c5c([2H])c([2H])c(-c6c([2H])c([2H])c([2H])c7c([2H])c([2H])c([2H])c([2H])c67)c6c([2H])c([2H])c([2H])c([2H])c56)c5c([2H])c([2H])c([2H])c([2H])c45)c([2H])c32)c1[2H]]

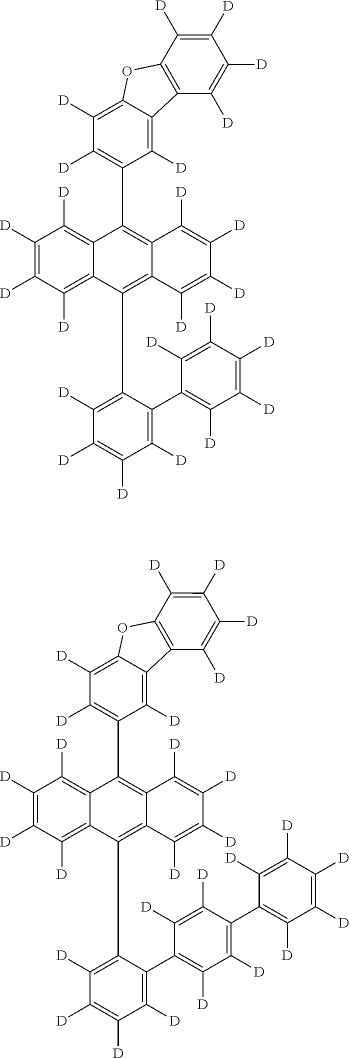 [SMILES: [2H]c1c([2H])c([2H])c(-c2c([2H])c([2H])c([2H])c([2H])c2-c2c3c([2H])c([2H])c([2H])c([2H])c3c(-c3c([2H])c([2H])c4oc5c([2H])c([2H])c([2H])c([2H])c5c4c3[2H])c3c([2H])c([2H])c([2H])c([2H])c23)c([2H])c1[2H] ; [2H]c1c([2H])c([2H])c(-c2c([2H])c([2H])c(-c3c([2H])c([2H])c([2H])c([2H])c3-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c([2H])c2[2H])c([2H])c1[2H]]

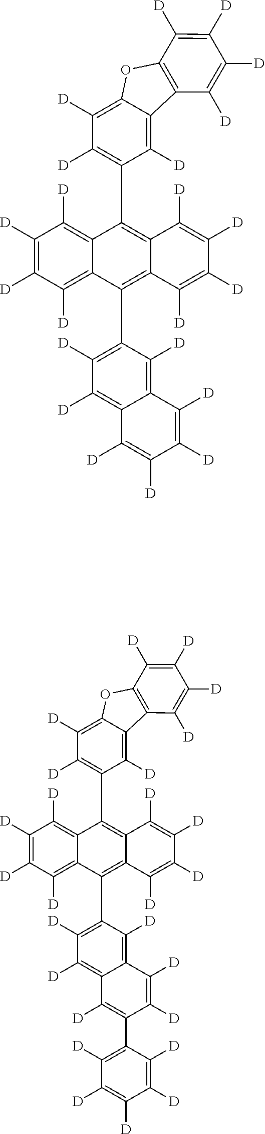 [SMILES: [2H]c1c([2H])c([2H])c2c(oc3c([2H])c([2H])c(-c4c5c([2H])c([2H])c([2H])c([2H])c5c(-c5c([2H])c([2H])c6c([2H])c([2H])c([2H])c([2H])c6c5[2H])c5c([2H])c([2H])c([2H])c([2H])c45)c([2H])c32)c1[2H] ; [2H]c1c([2H])c([2H])c(-c2c([2H])c([2H])c3c([2H])c(-c4c5c([2H])c([2H])c([2H])c([2H])c5c(-c5c([2H])c([2H])c6oc7c([2H])c([2H])c([2H])c([2H])c7c6c5[2H])c5c([2H])c([2H])c([2H])c([2H])c45)c([2H])c([2H])c3c2[2H])c([2H])c1[2H]]

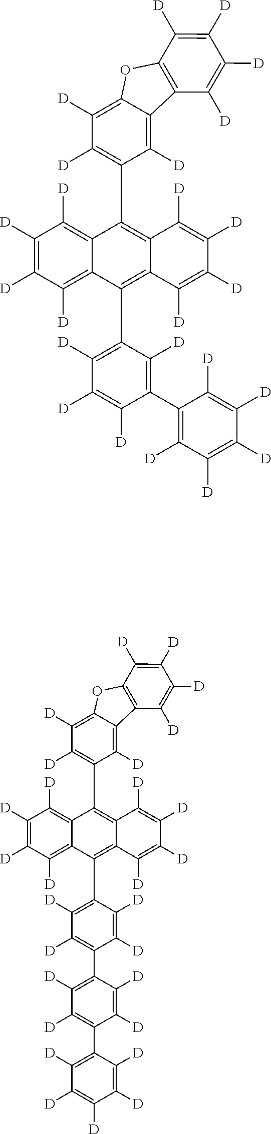 [SMILES: [2H]c1c([2H])c([2H])c(-c2c([2H])c([2H])c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c2[2H])c([2H])c1[2H] ; [2H]c1c([2H])c([2H])c(-c2c([2H])c([2H])c(-c3c([2H])c([2H])c(-c4c5c([2H])c([2H])c([2H])c([2H])c5c(-c5c([2H])c([2H])c6oc7c([2H])c([2H])c([2H])c([2H])c7c6c5[2H])c5c([2H])c([2H])c([2H])c([2H])c45)c([2H])c3[2H])c([2H])c2[2H])c([2H])c1[2H]]

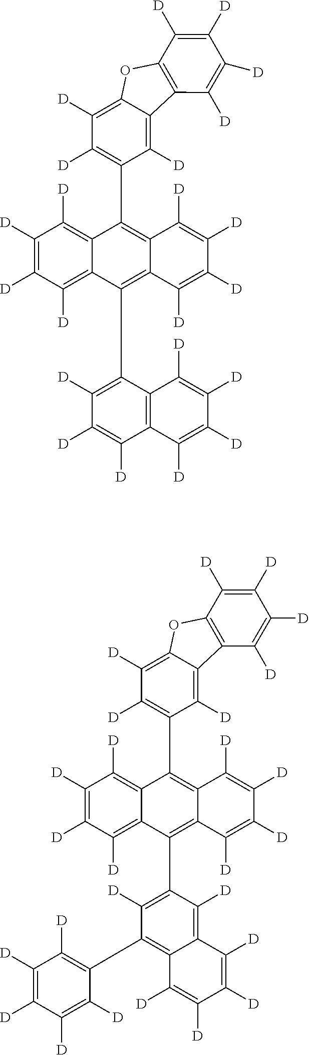 [SMILES: [2H]c1c([2H])c([2H])c2c(oc3c([2H])c([2H])c(-c4c5c([2H])c([2H])c([2H])c([2H])c5c(-c5c([2H])c([2H])c([2H])c6c([2H])c([2H])c([2H])c([2H])c56)c5c([2H])c([2H])c([2H])c([2H])c45)c([2H])c32)c1[2H] ; [2H]c1c([2H])c([2H])c(-c2c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c([2H])c3c([2H])c([2H])c([2H])c([2H])c23)c([2H])c1[2H]]

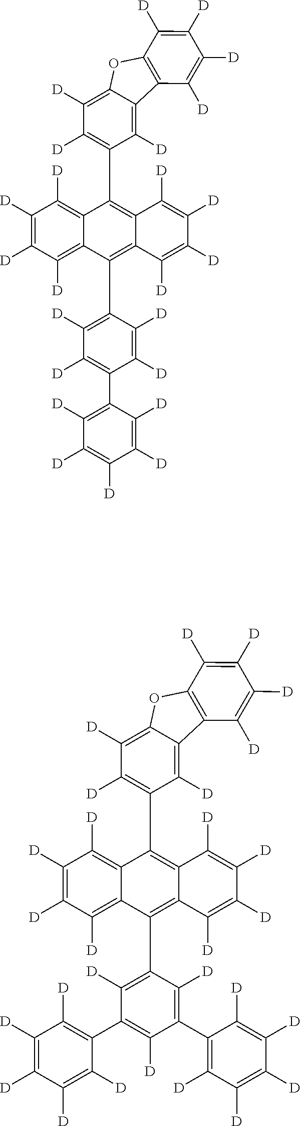 [SMILES: [2H]c1c([2H])c([2H])c(-c2c([2H])c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c([2H])c2[2H])c([2H])c1[2H] ; [2H]c1c([2H])c([2H])c(-c2c([2H])c(-c3c([2H])c([2H])c([2H])c([2H])c3[2H])c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c2[2H])c([2H])c1[2H]]

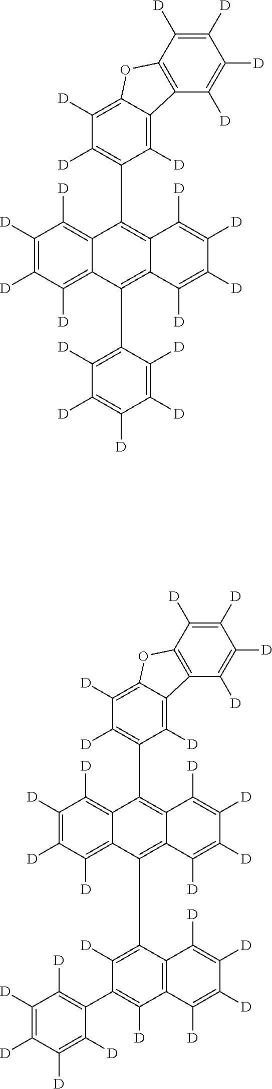 [SMILES: [2H]c1c([2H])c([2H])c(-c2c3c([2H])c([2H])c([2H])c([2H])c3c(-c3c([2H])c([2H])c4oc5c([2H])c([2H])c([2H])c([2H])c5c4c3[2H])c3c([2H])c([2H])c([2H])c([2H])c23)c([2H])c1[2H] ; [2H]c1c([2H])c([2H])c(-c2c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c3c([2H])c([2H])c([2H])c([2H])c3c2[2H])c([2H])c1[2H]]

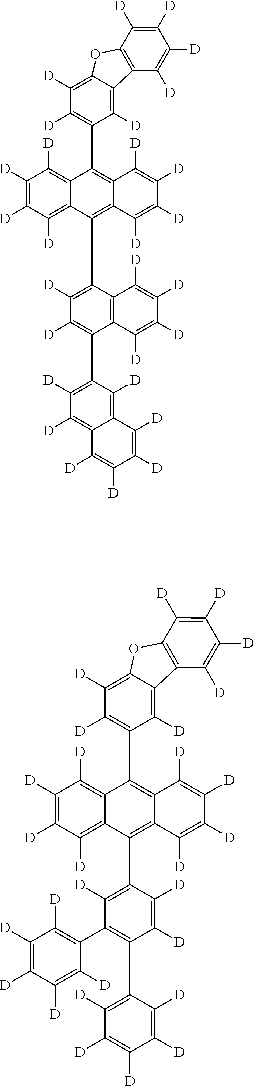 [SMILES: [2H]c1c([2H])c([2H])c2c(oc3c([2H])c([2H])c(-c4c5c([2H])c([2H])c([2H])c([2H])c5c(-c5c([2H])c([2H])c(-c6c([2H])c([2H])c7c([2H])c([2H])c([2H])c([2H])c7c6[2H])c6c([2H])c([2H])c([2H])c([2H])c56)c5c([2H])c([2H])c([2H])c([2H])c45)c([2H])c32)c1[2H] ; [2H]c1c([2H])c([2H])c(-c2c([2H])c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c([2H])c2-c2c([2H])c([2H])c([2H])c([2H])c2[2H])c([2H])c1[2H]]

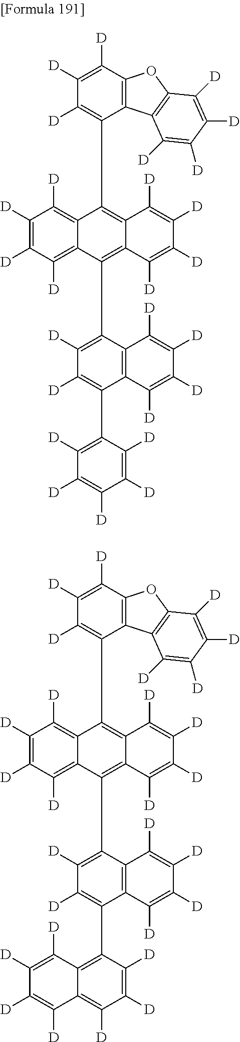

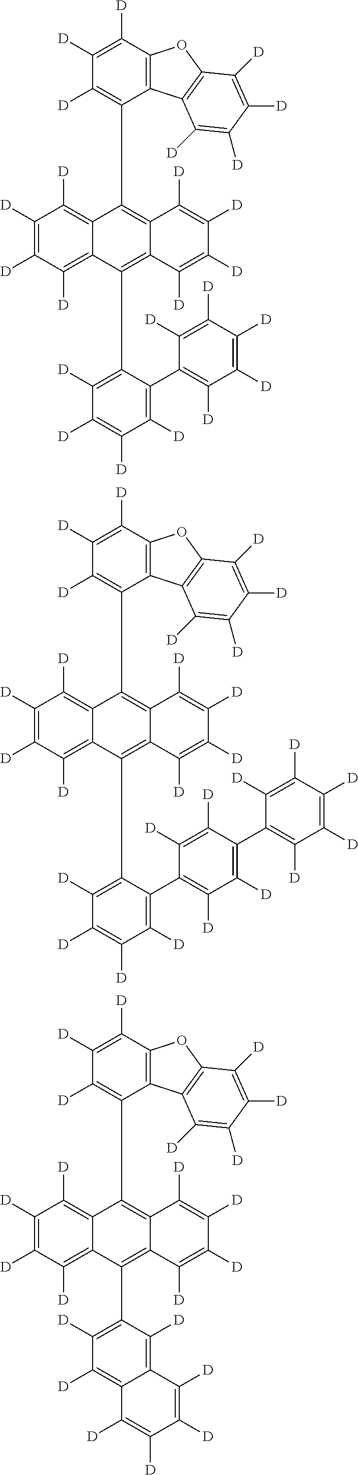

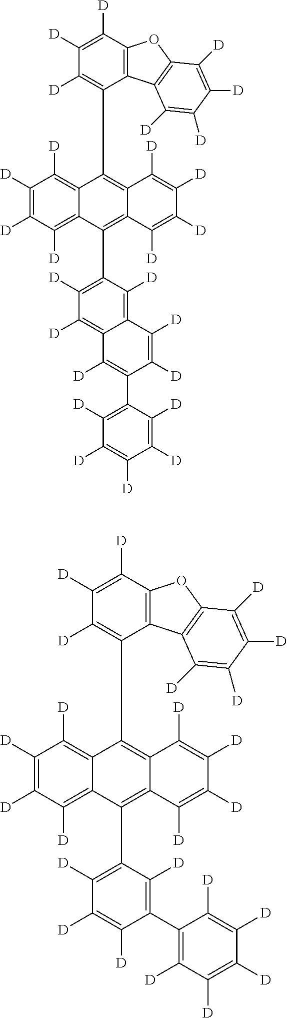 [SMILES: [2H]c1c([2H])c([2H])c(-c2c([2H])c([2H])c3c([2H])c(-c4c5c([2H])c([2H])c([2H])c([2H])c5c(-c5c([2H])c([2H])c([2H])c6oc7c([2H])c([2H])c([2H])c([2H])c7c56)c5c([2H])c([2H])c([2H])c([2H])c45)c([2H])c([2H])c3c2[2H])c([2H])c1[2H] ; [2H]c1c([2H])c([2H])c(-c2c([2H])c([2H])c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c2[2H])c([2H])c1[2H]]

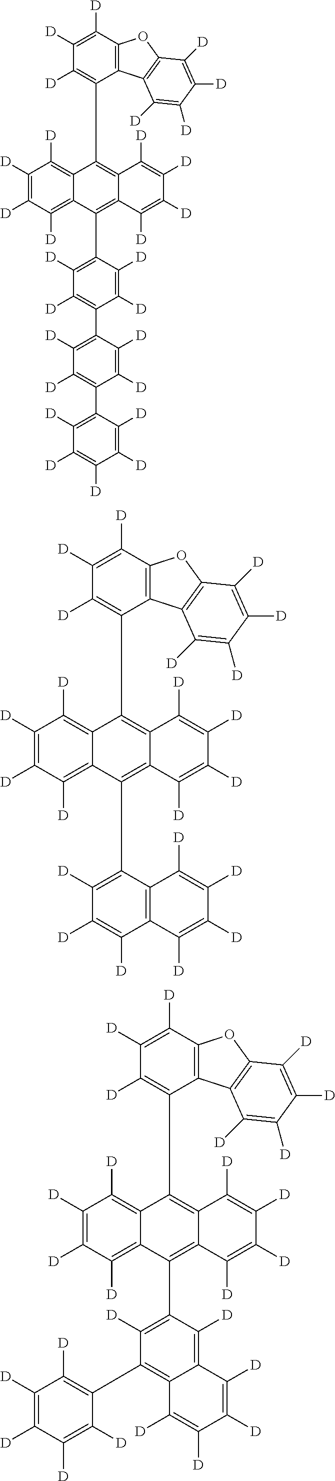

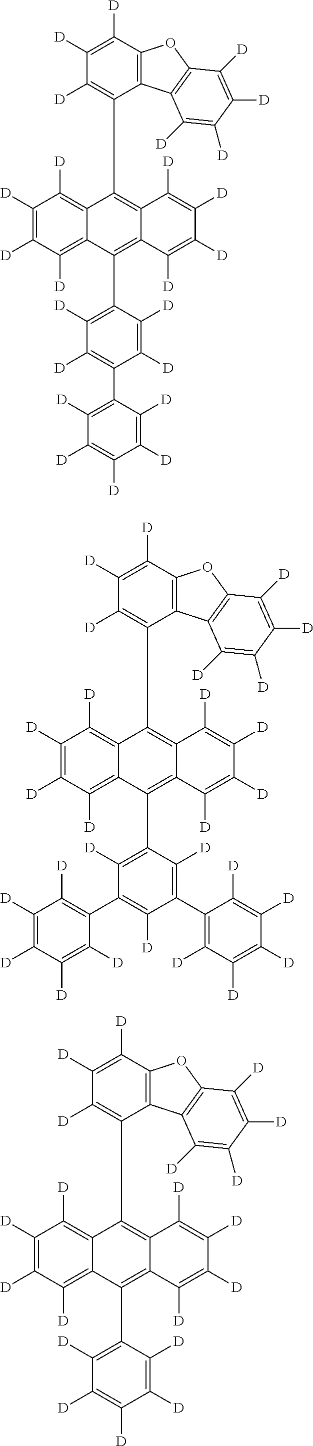

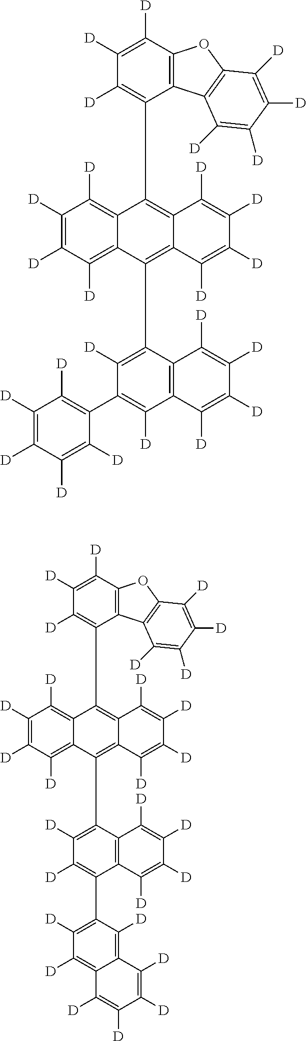 [SMILES: [2H]c1c([2H])c([2H])c(-c2c([2H])c(-c3c4c([2H])c([2H])c([2H])c([2H])c4c(-c4c([2H])c([2H])c([2H])c5oc6c([2H])c([2H])c([2H])c([2H])c6c45)c4c([2H])c([2H])c([2H])c([2H])c34)c3c([2H])c([2H])c([2H])c([2H])c3c2[2H])c([2H])c1[2H] ; [2H]c1c([2H])c([2H])c2c(oc3c([2H])c([2H])c([2H])c(-c4c5c([2H])c([2H])c([2H])c([2H])c5c(-c5c([2H])c([2H])c(-c6c([2H])c([2H])c7c([2H])c([2H])c([2H])c([2H])c7c6[2H])c6c([2H])c([2H])c([2H])c([2H])c56)c5c([2H])c([2H])c([2H])c([2H])c45)c32)c1[2H]]

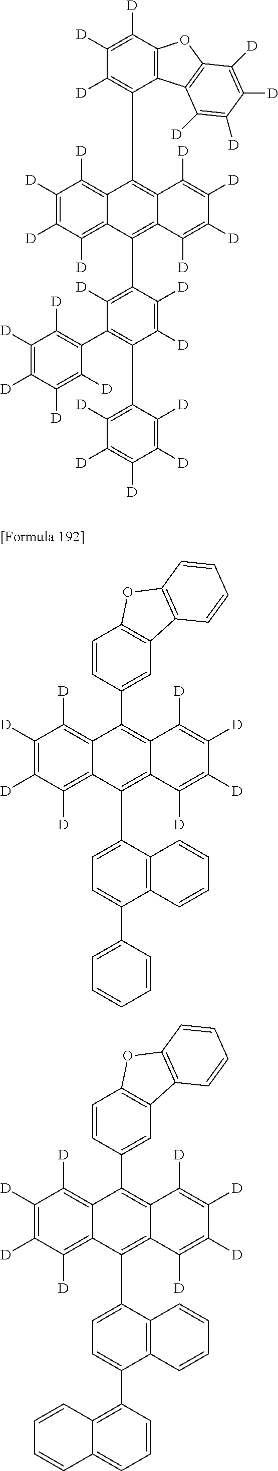

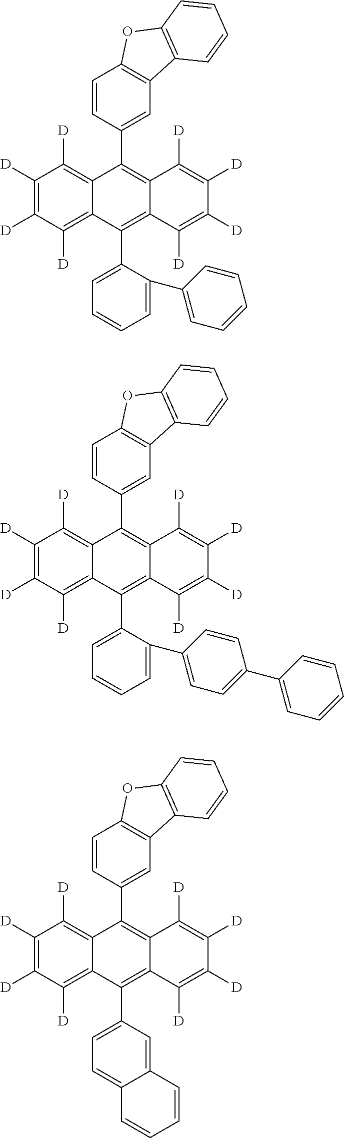

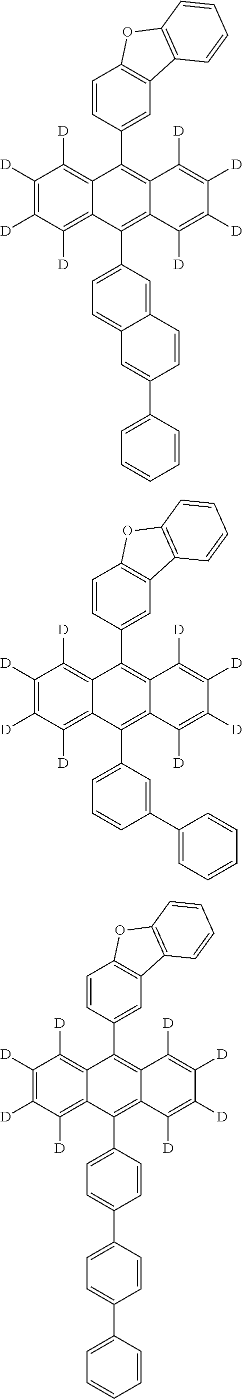

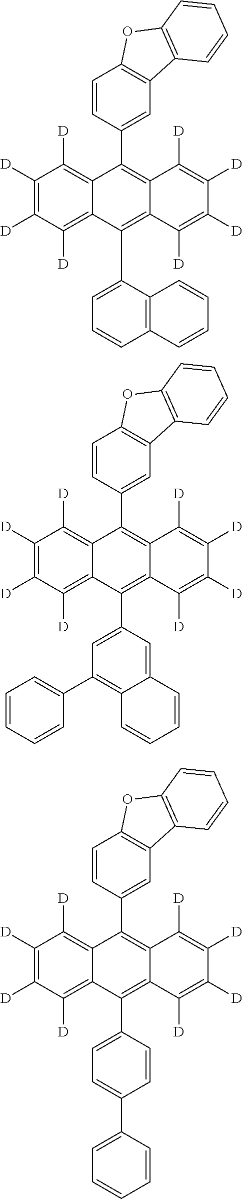

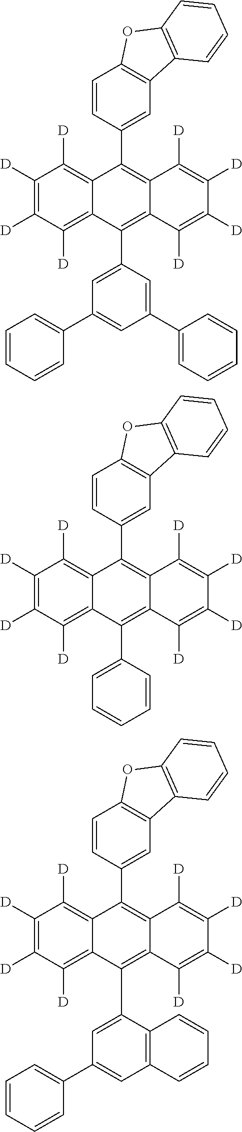

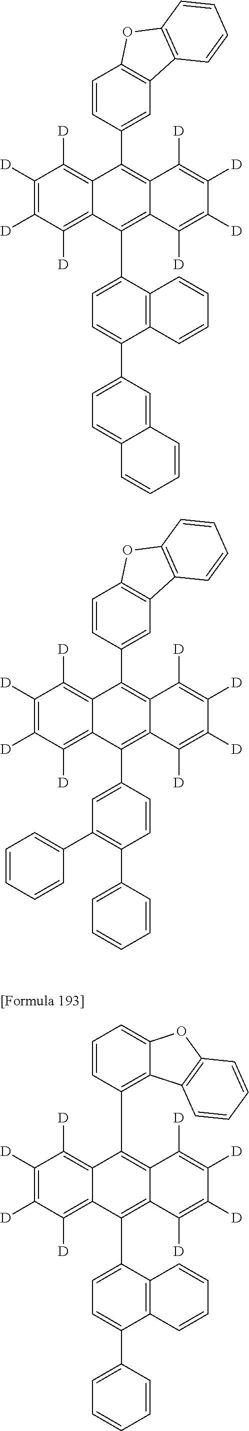

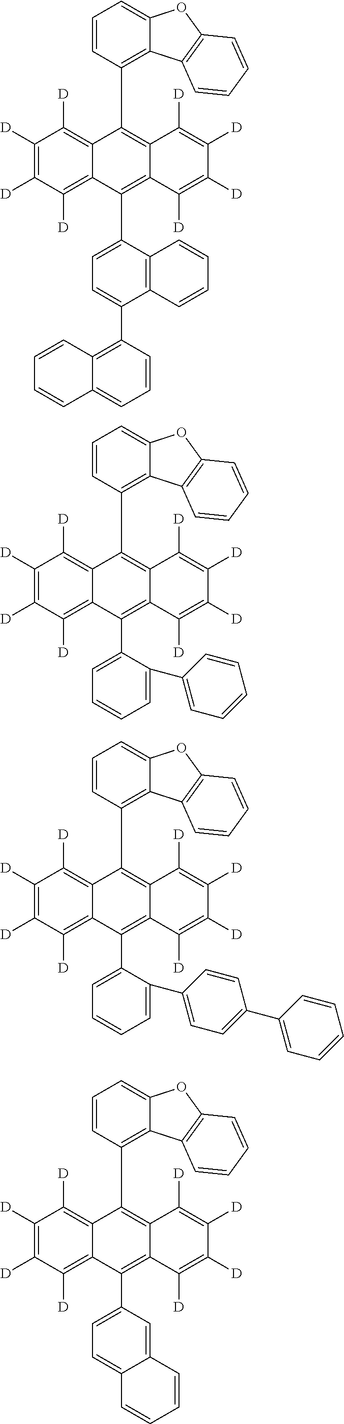

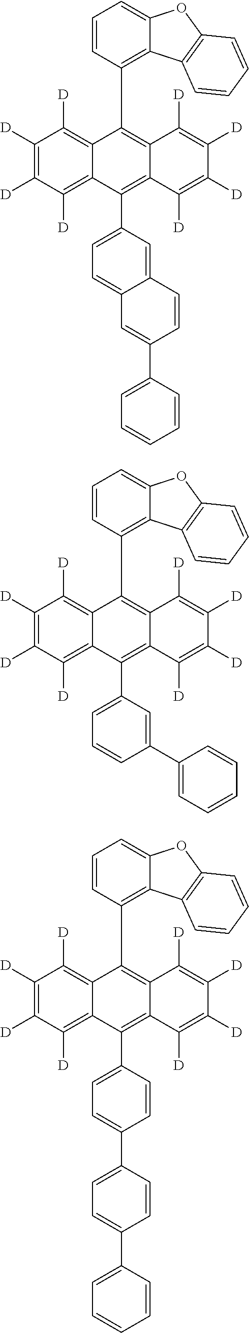

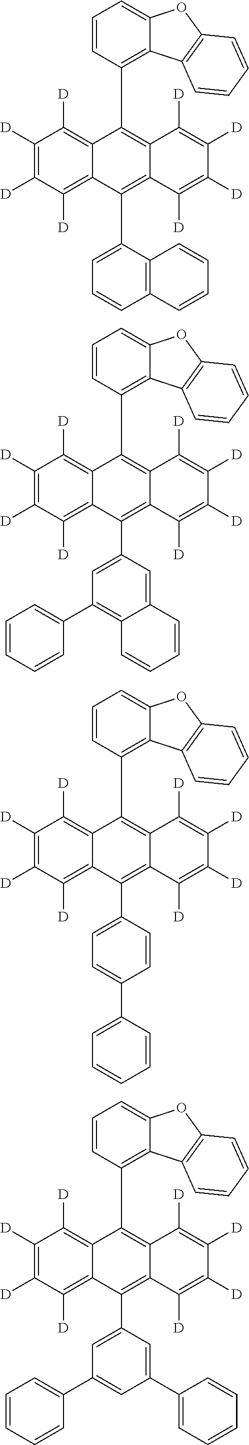

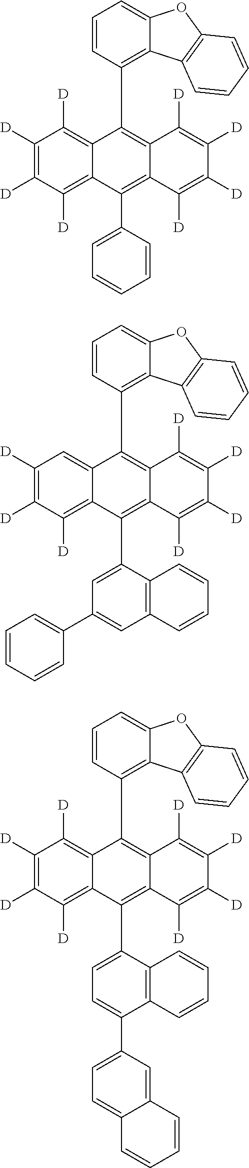

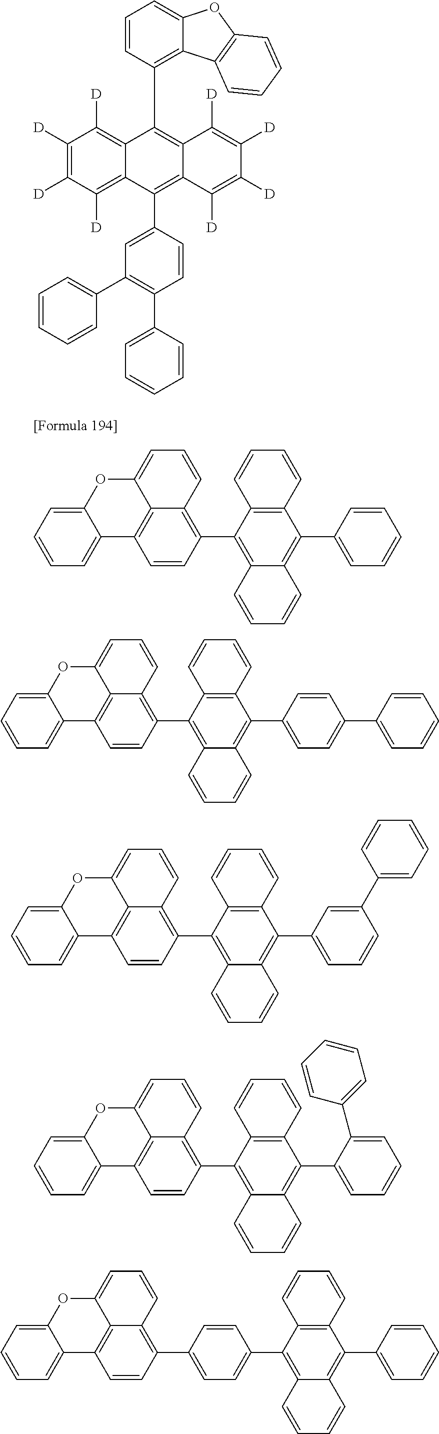

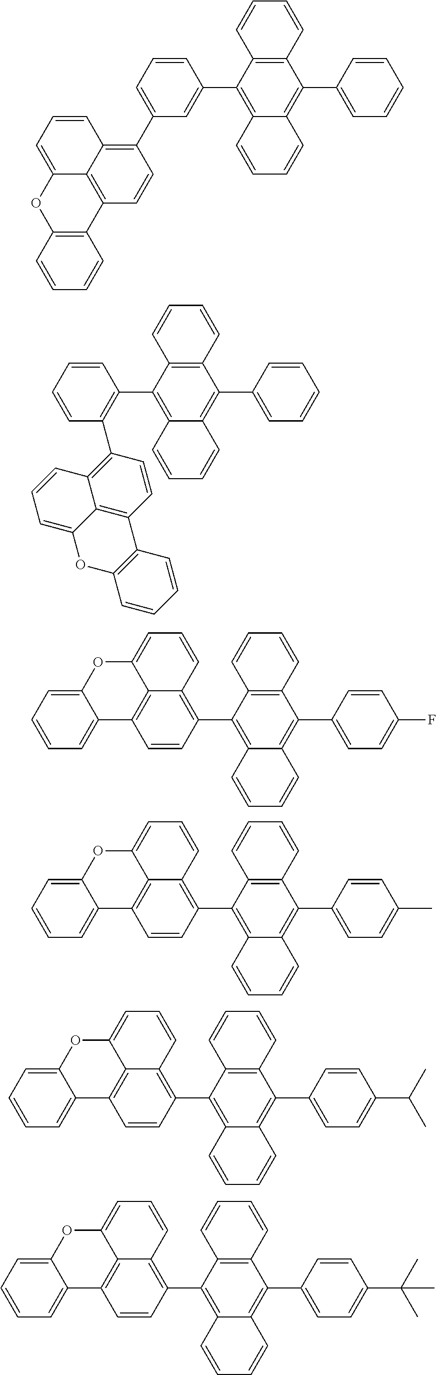

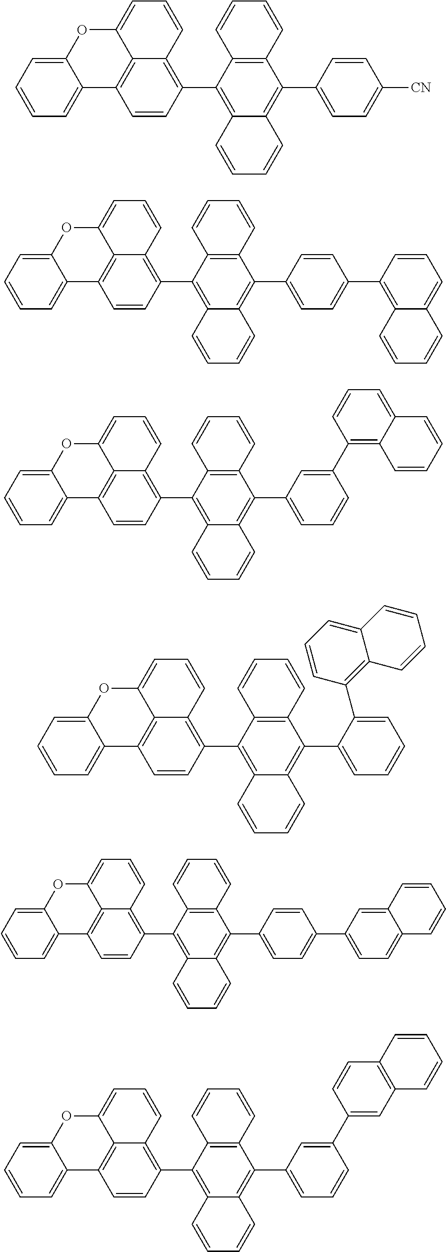

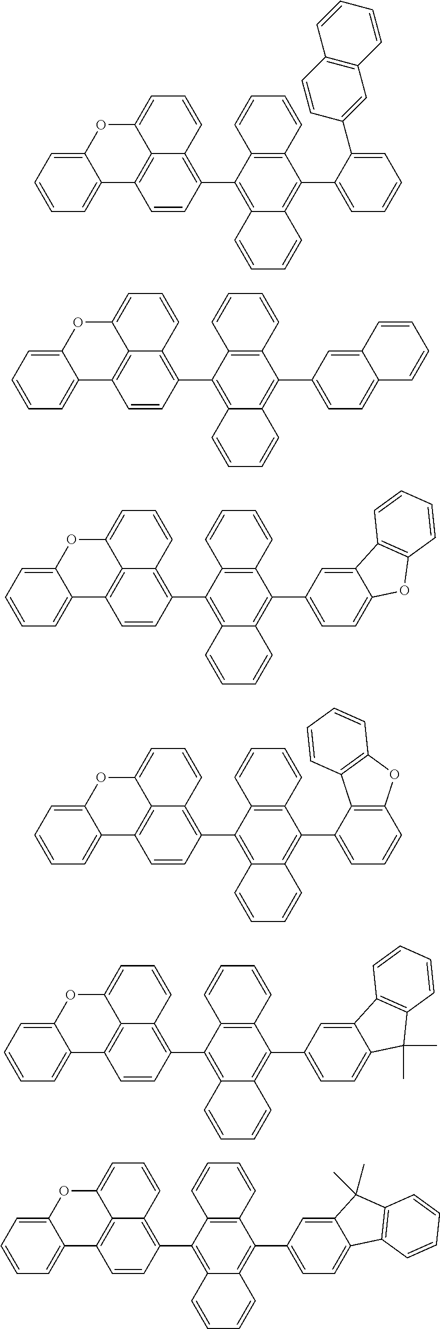

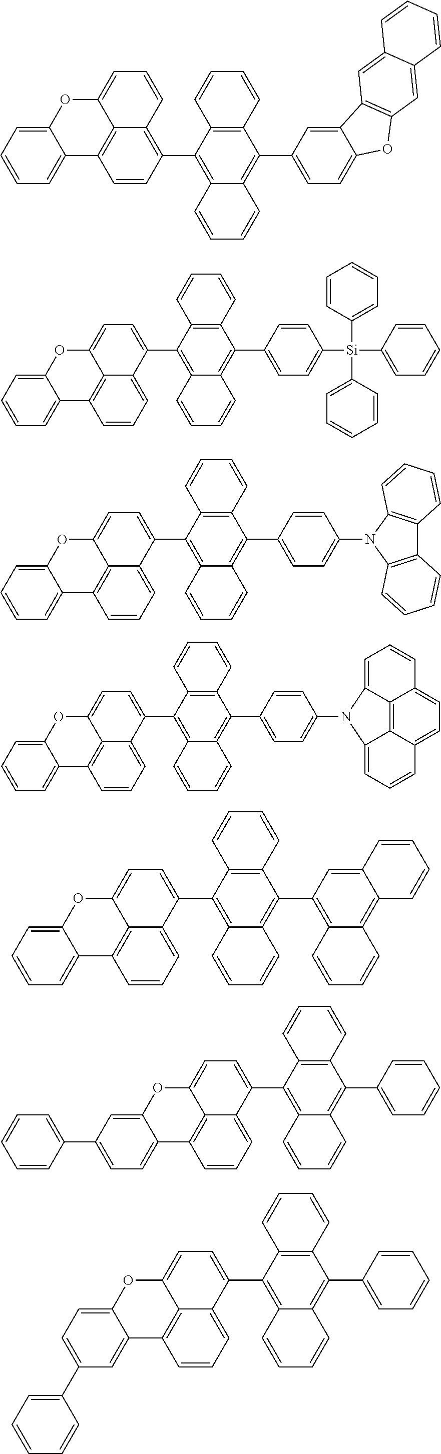

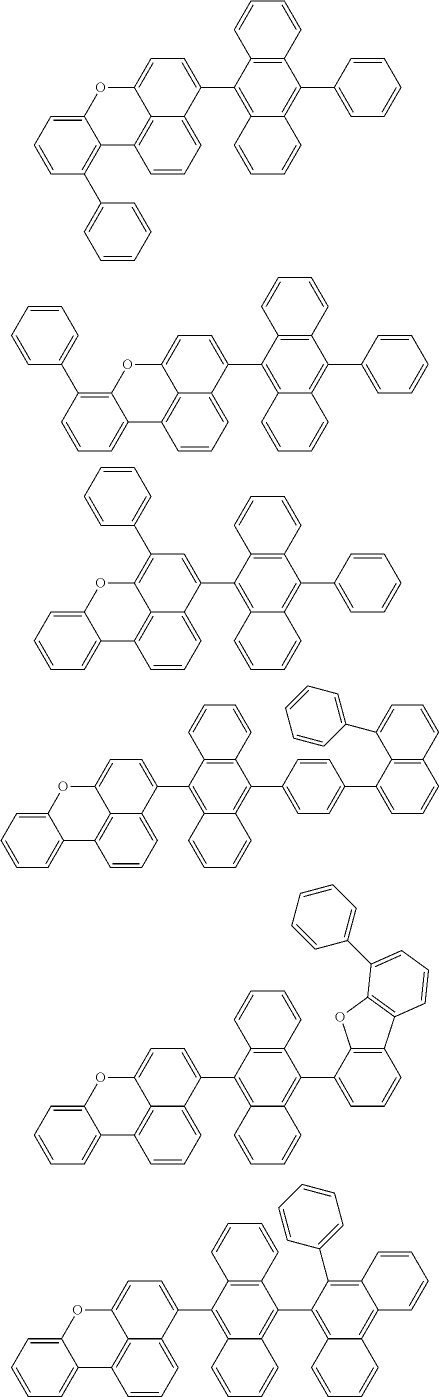

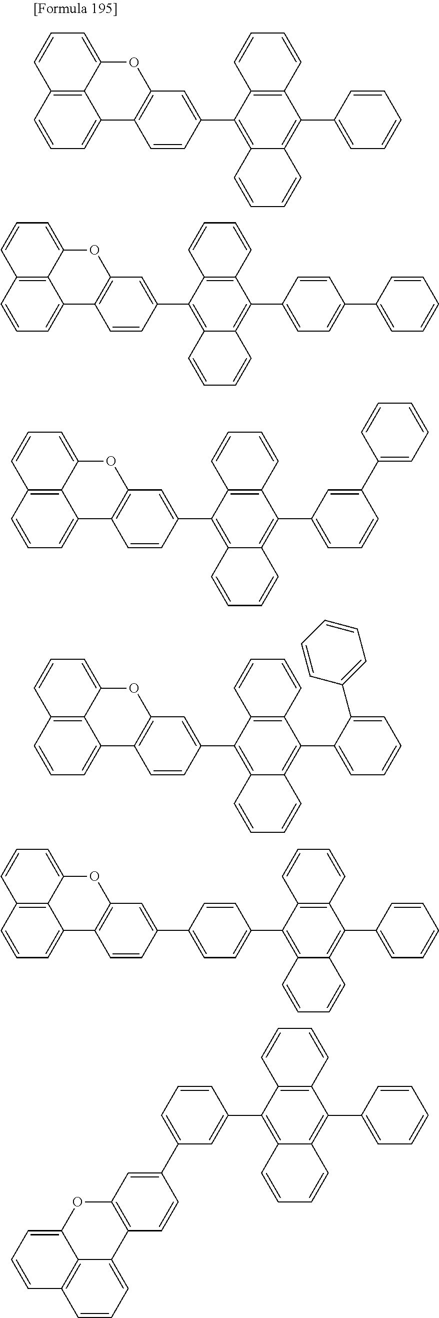

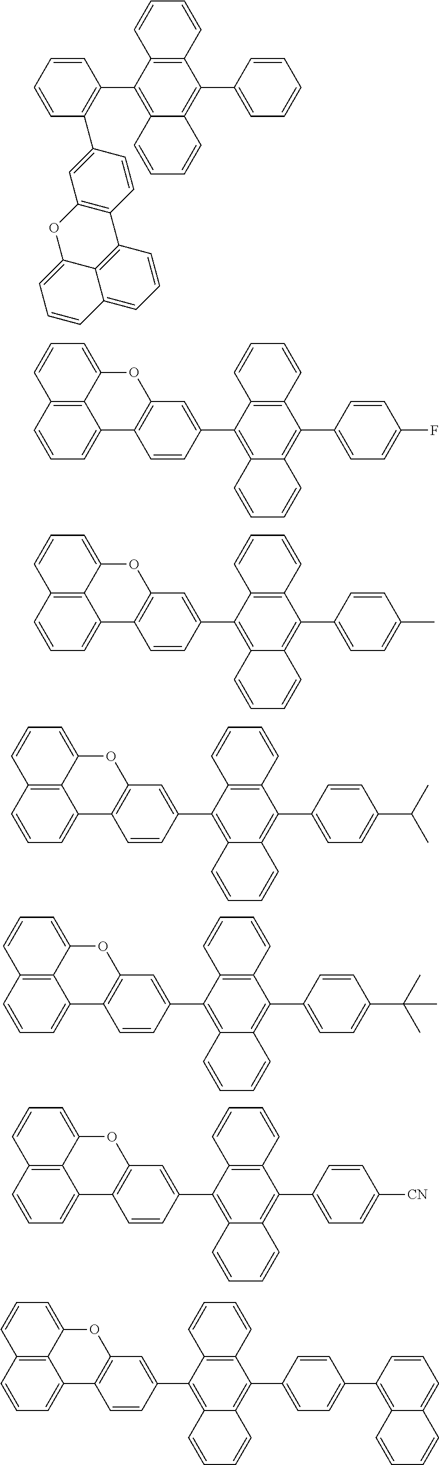

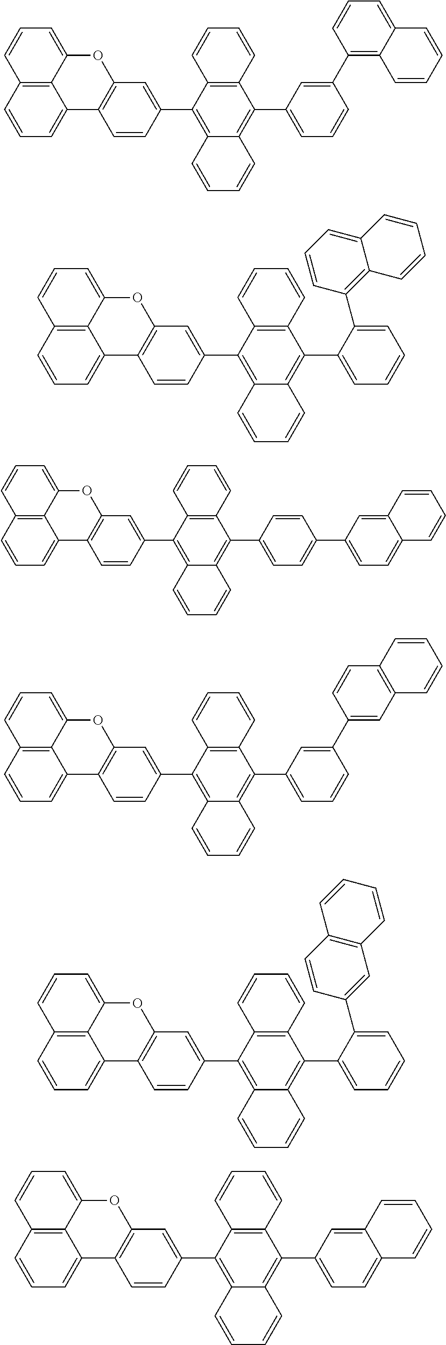

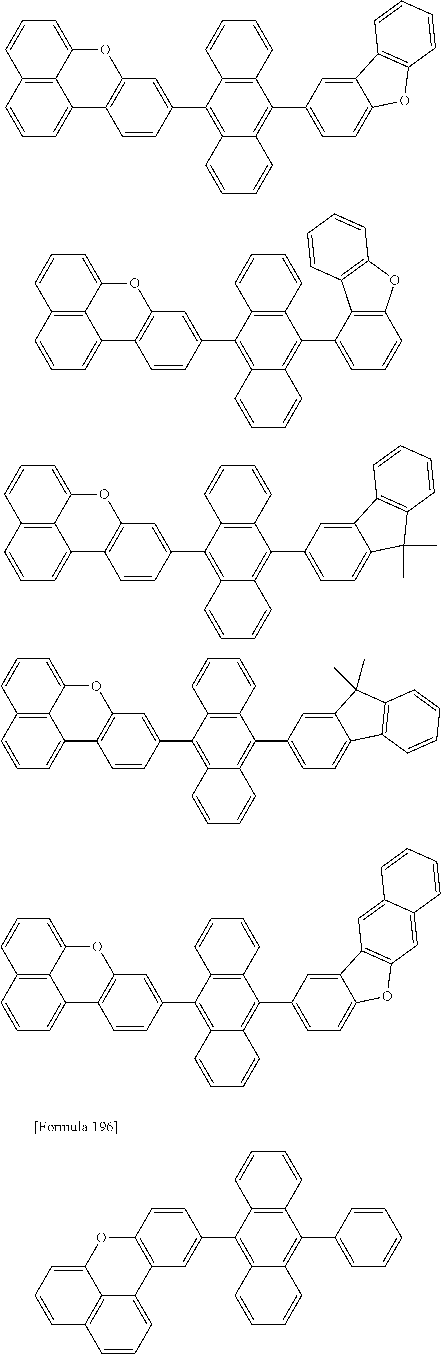

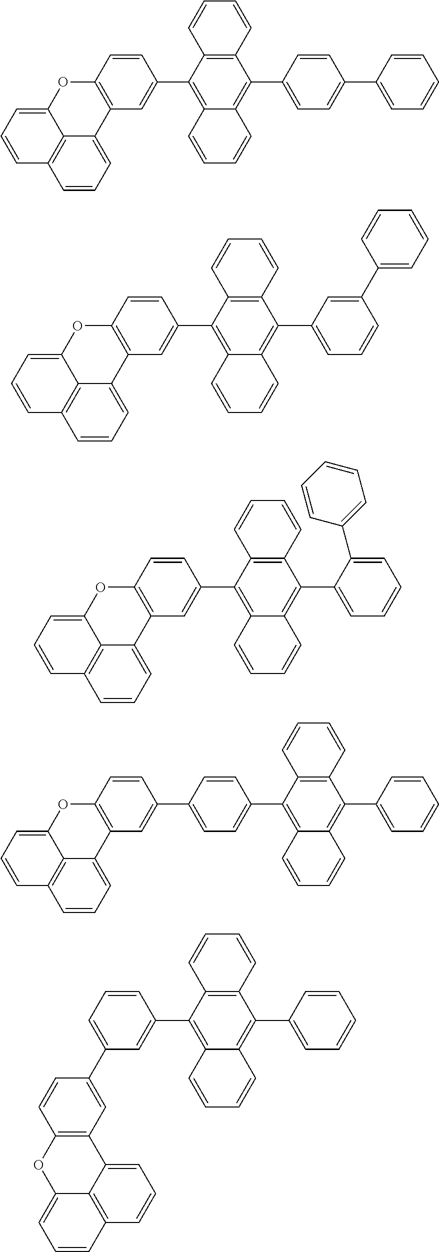 [SMILES: c1ccc(-c2ccc(-c3c4ccccc4c(-c4ccc5c(c4)-c4cccc6cccc(c46)O5)c4ccccc34)cc2)cc1 ; c1ccc(-c2cccc(-c3c4ccccc4c(-c4ccc5c(c4)-c4cccc6cccc(c46)O5)c4ccccc34)c2)cc1 ; c1ccc(-c2ccccc2-c2c3ccccc3c(-c3ccc4c(c3)-c3cccc5cccc(c35)O4)c3ccccc23)cc1 ; c1ccc(-c2c3ccccc3c(-c3ccc(-c4ccc5c(c4)-c4cccc6cccc(c46)O5)cc3)c3ccccc23)cc1 ; c1ccc(-c2c3ccccc3c(-c3cccc(-c4ccc5c(c4)-c4cccc6cccc(c46)O5)c3)c3ccccc23)cc1]

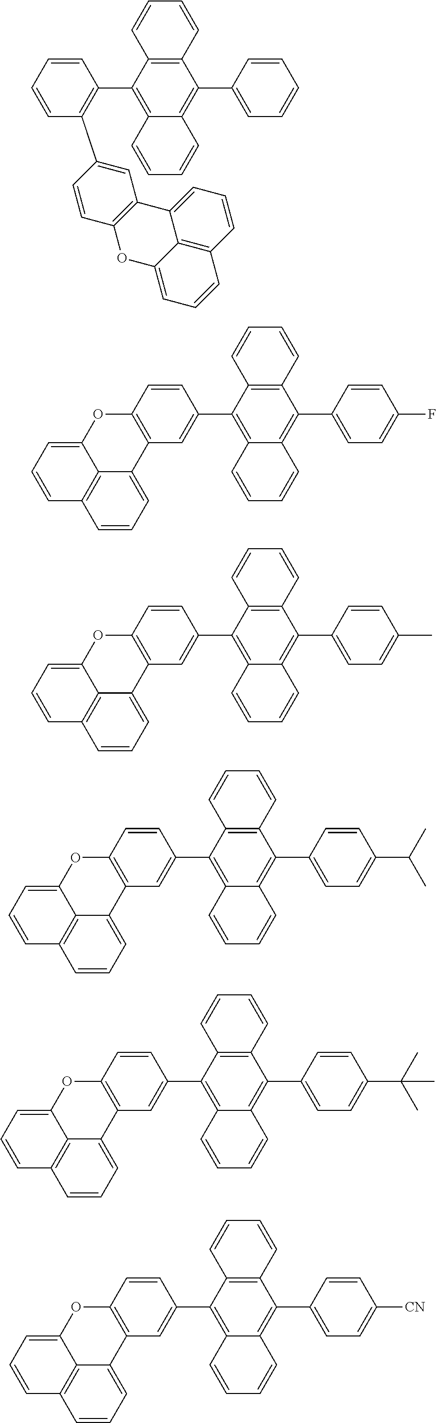 [SMILES: c1ccc(-c2c3ccccc3c(-c3ccccc3-c3ccc4c(c3)-c3cccc5cccc(c35)O4)c3ccccc23)cc1 ; Fc1ccc(-c2c3ccccc3c(-c3ccc4c(c3)-c3cccc5cccc(c35)O4)c3ccccc23)cc1 ; Cc1ccc(-c2c3ccccc3c(-c3ccc4c(c3)-c3cccc5cccc(c35)O4)c3ccccc23)cc1 ; CC(C)c1ccc(-c2c3ccccc3c(-c3ccc4c(c3)-c3cccc5cccc(c35)O4)c3ccccc23)cc1 ; CC(C)(C)c1ccc(-c2c3ccccc3c(-c3ccc4c(c3)-c3cccc5cccc(c35)O4)c3ccccc23)cc1 ; N#Cc1ccc(-c2c3ccccc3c(-c3ccc4c(c3)-c3cccc5cccc(c35)O4)c3ccccc23)cc1]

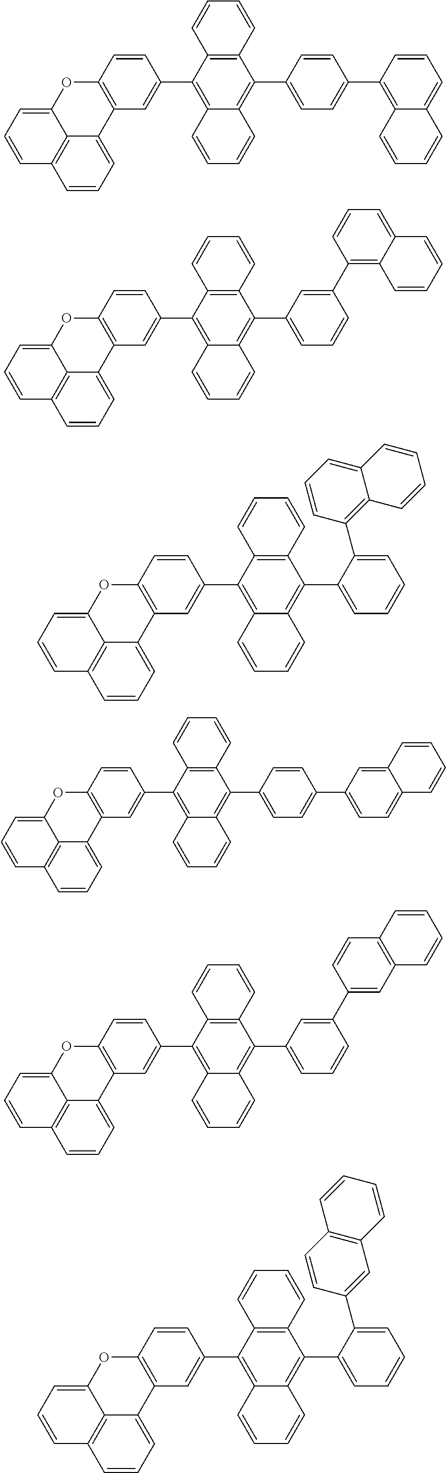

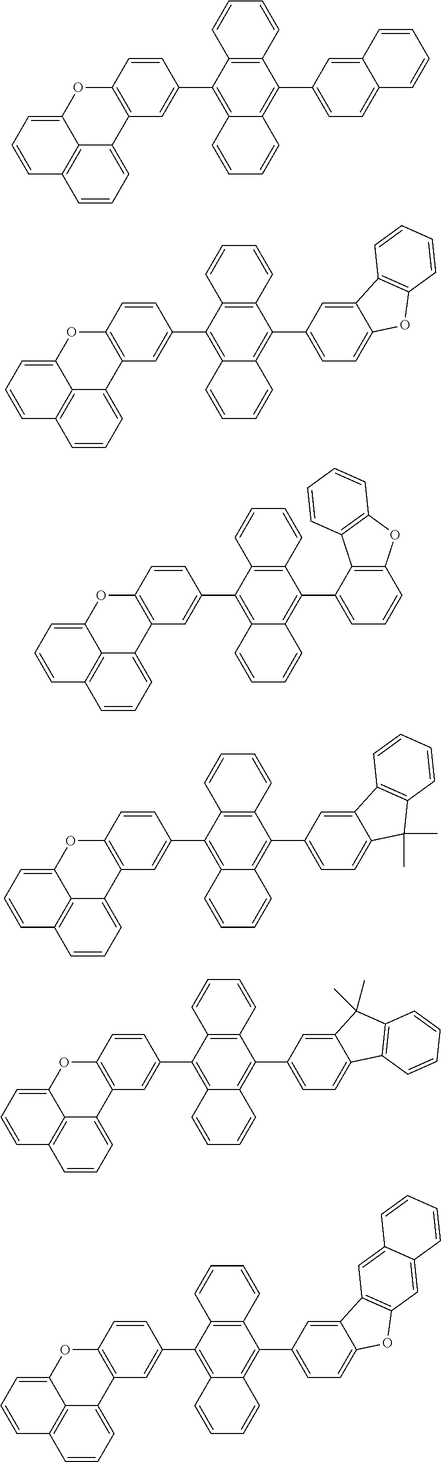 [SMILES: c1ccc2cc(-c3c4ccccc4c(-c4ccc5c(c4)-c4cccc6cccc(c46)O5)c4ccccc34)ccc2c1 ; c1cc2c3c(cccc3c1)-c1cc(-c3c4ccccc4c(-c4ccc5oc6ccccc6c5c4)c4ccccc34)ccc1O2 ; c1cc2c3c(cccc3c1)-c1cc(-c3c4ccccc4c(-c4cccc5oc6ccccc6c45)c4ccccc34)ccc1O2 ; CC1(C)c2ccccc2-c2cc(-c3c4ccccc4c(-c4ccc5c(c4)-c4cccc6cccc(c46)O5)c4ccccc34)ccc21 ; CC1(C)c2ccccc2-c2ccc(-c3c4ccccc4c(-c4ccc5c(c4)-c4cccc6cccc(c46)O5)c4ccccc34)cc21 ; c1ccc2cc3c(cc2c1)oc1ccc(-c2c4ccccc4c(-c4ccc5c(c4)-c4cccc6cccc(c46)O5)c4ccccc24)cc13]

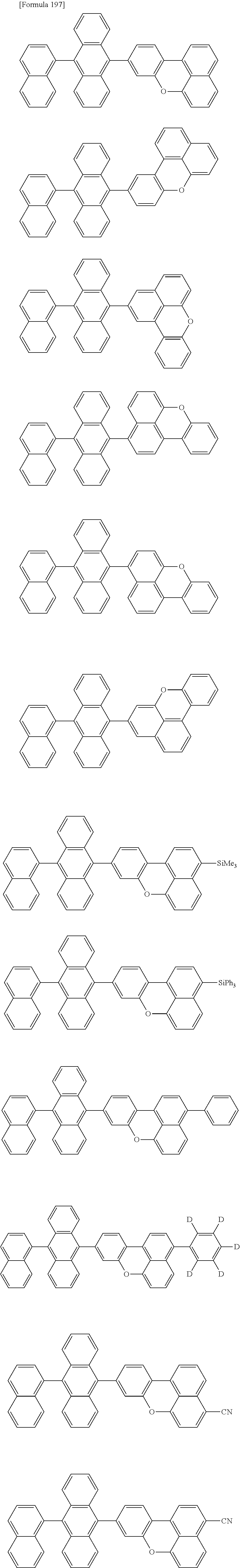

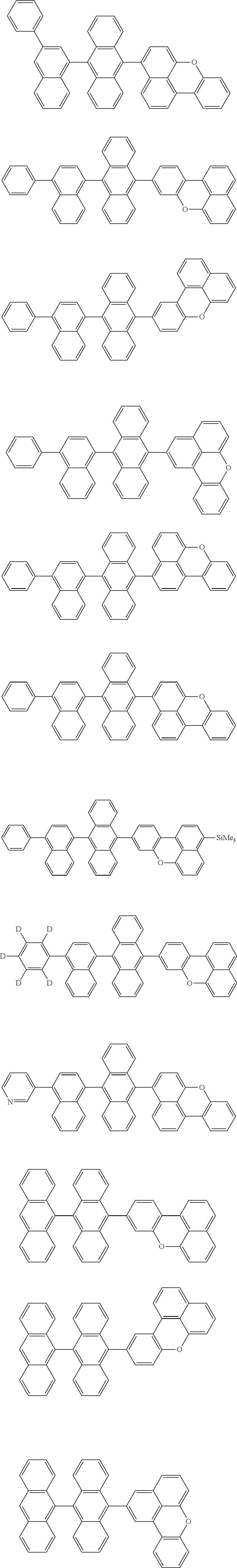

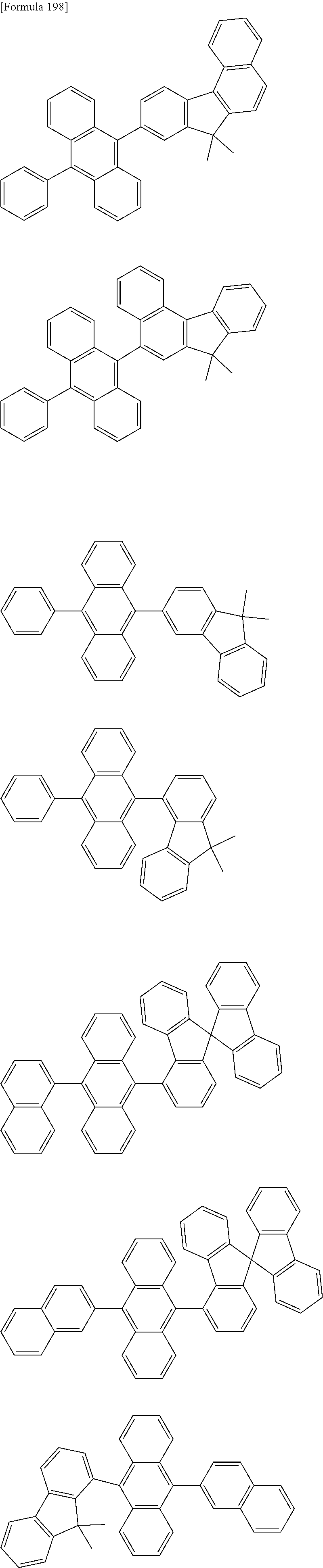

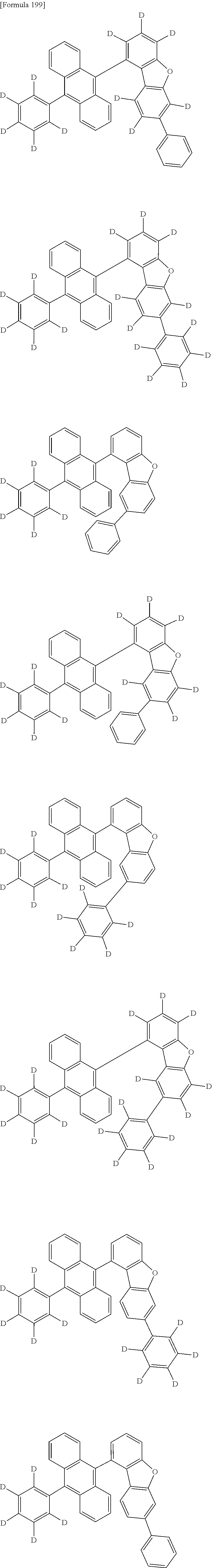

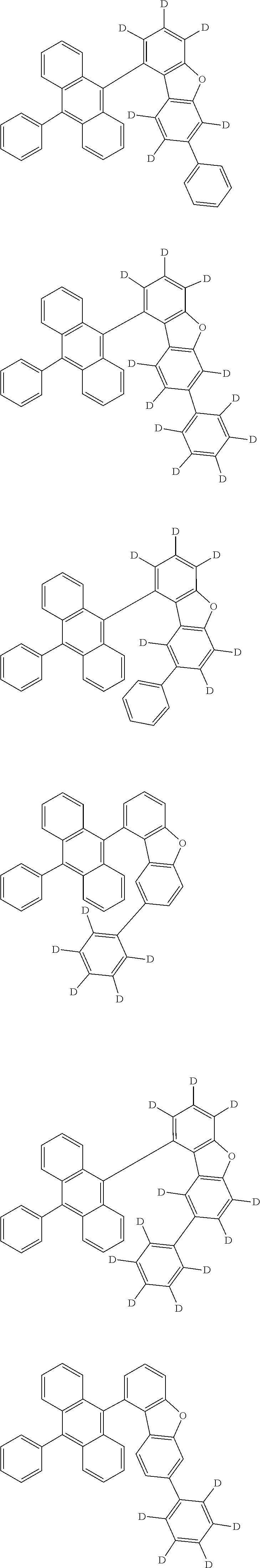

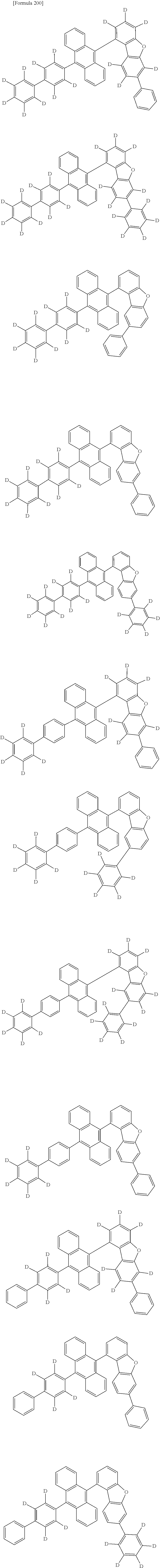

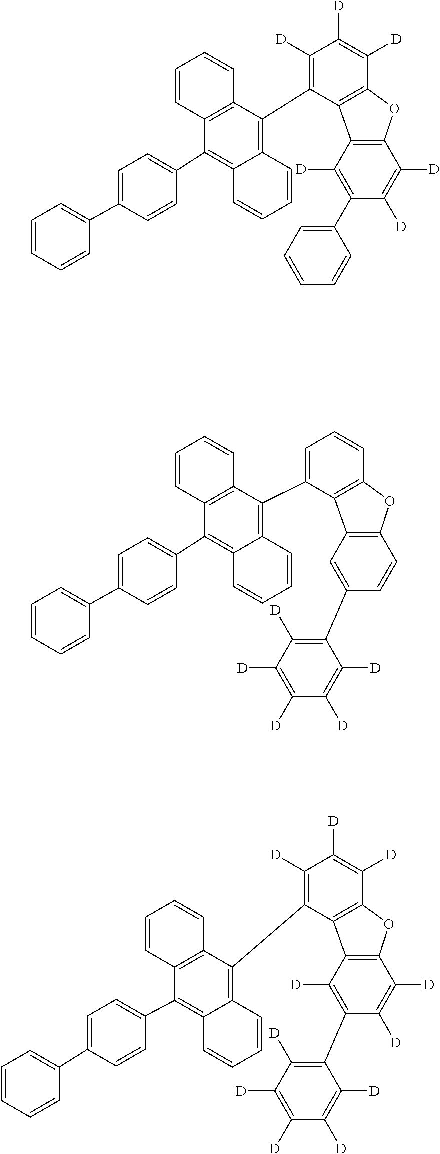 [SMILES: [2H]c1c(-c2ccccc2)c([2H])c2c(oc3c([2H])c([2H])c([2H])c(-c4c5ccccc5c(-c5ccc(-c6ccccc6)cc5)c5ccccc45)c32)c1[2H] ; [2H]c1c([2H])c([2H])c(-c2ccc3oc4cccc(-c5c6ccccc6c(-c6ccc(-c7ccccc7)cc6)c6ccccc56)c4c3c2)c([2H])c1[2H] ; [2H]c1c([2H])c([2H])c(-c2c([2H])c([2H])c3oc4c([2H])c([2H])c([2H])c(-c5c6ccccc6c(-c6ccc(-c7ccccc7)cc6)c6ccccc56)c4c3c2[2H])c([2H])c1[2H]]

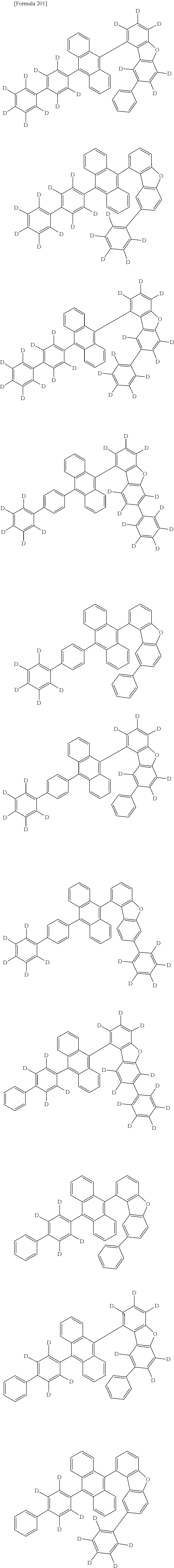

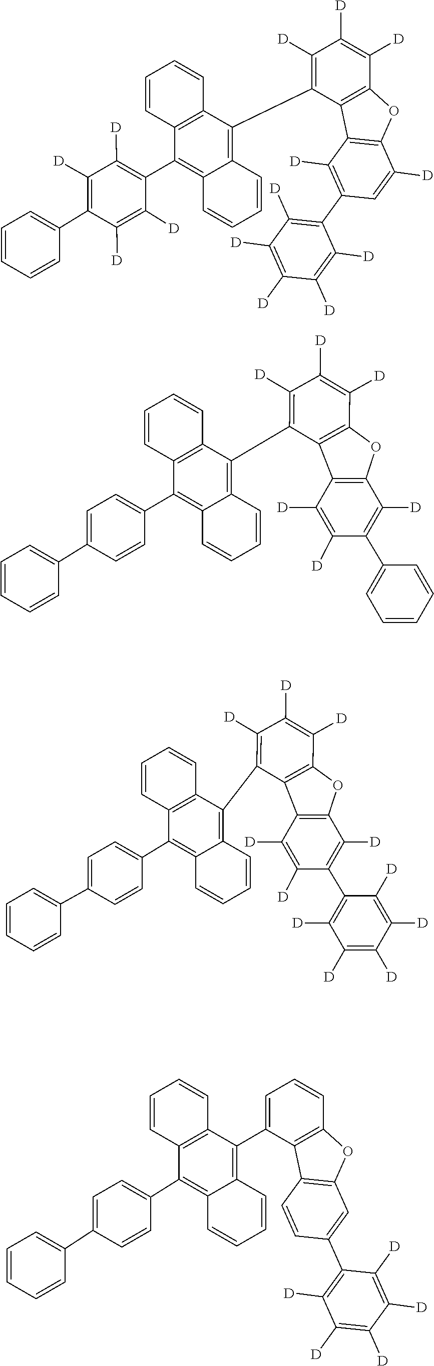

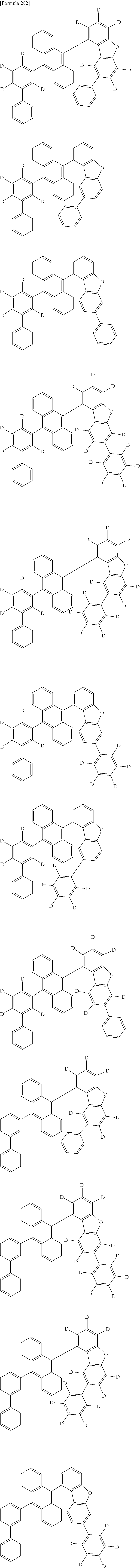

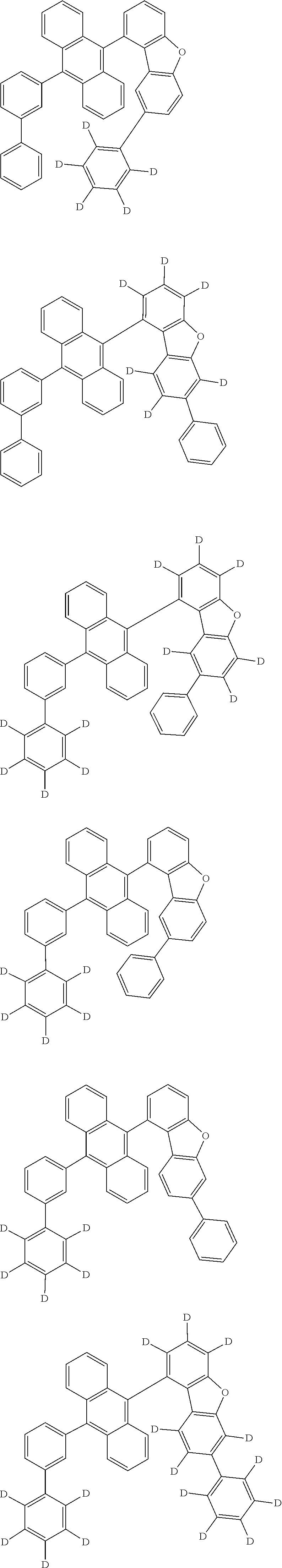

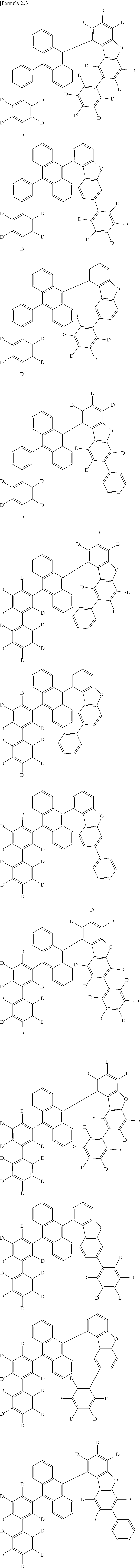

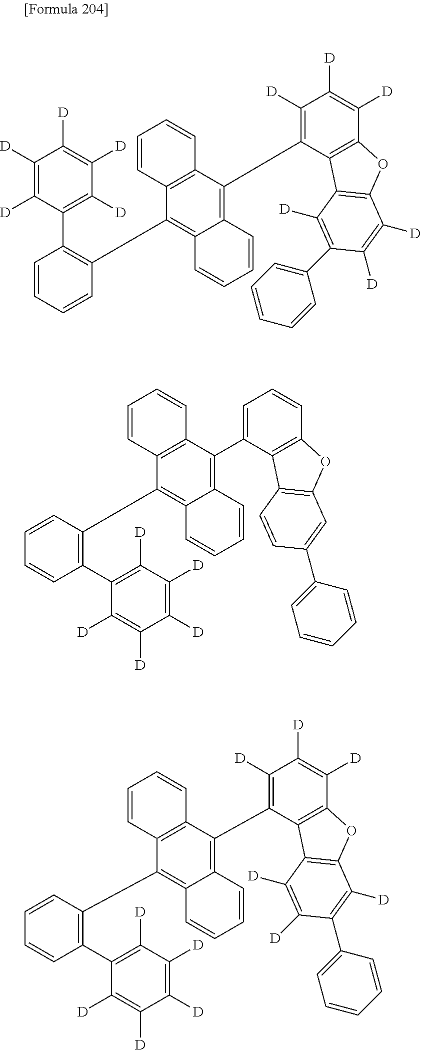 [SMILES: [2H]c1c([2H])c([2H])c(-c2ccccc2-c2c3ccccc3c(-c3c([2H])c([2H])c([2H])c4oc5c([2H])c([2H])c(-c6ccccc6)c([2H])c5c34)c3ccccc23)c([2H])c1[2H] ; [2H]c1c([2H])c([2H])c(-c2ccccc2-c2c3ccccc3c(-c3cccc4oc5cc(-c6ccccc6)ccc5c34)c3ccccc23)c([2H])c1[2H] ; [2H]c1c([2H])c([2H])c(-c2ccccc2-c2c3ccccc3c(-c3c([2H])c([2H])c([2H])c4oc5c([2H])c(-c6ccccc6)c([2H])c([2H])c5c34)c3ccccc23)c([2H])c1[2H]]

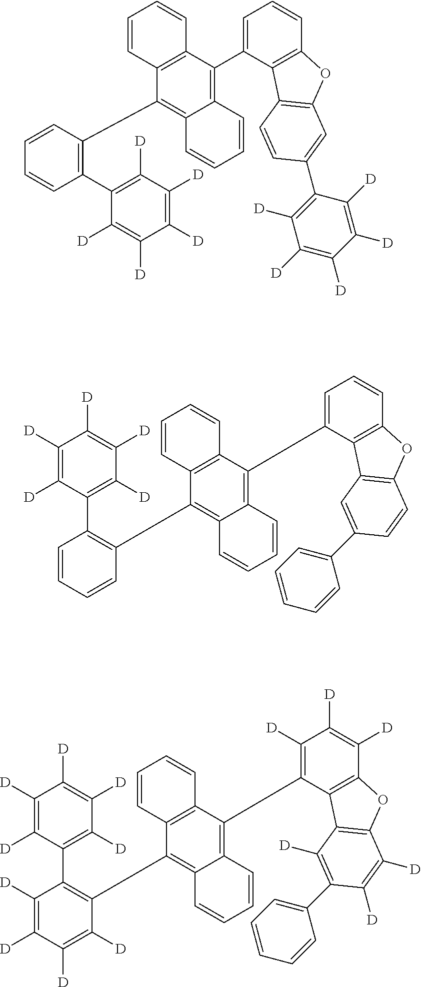 [SMILES: [2H]c1c([2H])c([2H])c(-c2ccc3c(c2)oc2cccc(-c4c5ccccc5c(-c5ccccc5-c5c([2H])c([2H])c([2H])c([2H])c5[2H])c5ccccc45)c23)c([2H])c1[2H] ; [2H]c1c([2H])c([2H])c(-c2ccccc2-c2c3ccccc3c(-c3cccc4oc5ccc(-c6ccccc6)cc5c34)c3ccccc23)c([2H])c1[2H] ; [2H]c1c([2H])c([2H])c(-c2c([2H])c([2H])c([2H])c([2H])c2-c2c3ccccc3c(-c3c([2H])c([2H])c([2H])c4oc5c([2H])c([2H])c(-c6ccccc6)c([2H])c5c34)c3ccccc23)c([2H])c1[2H]]

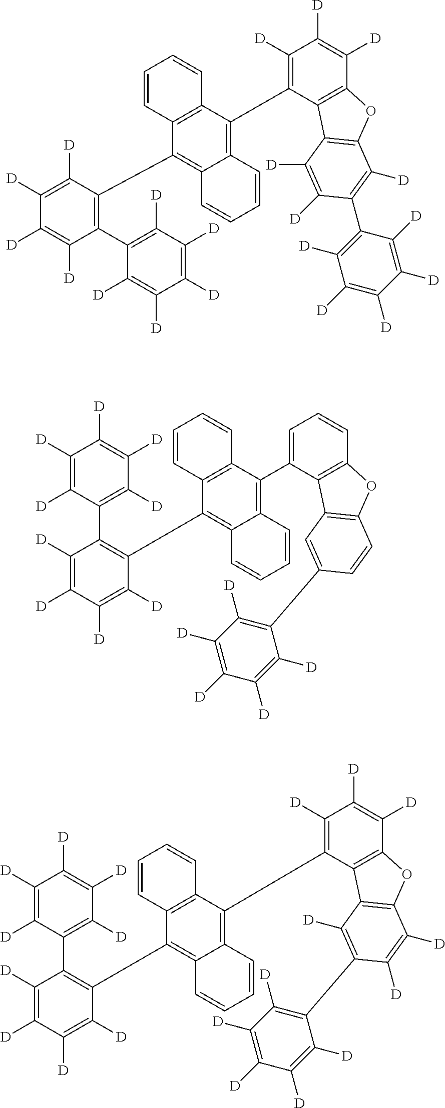

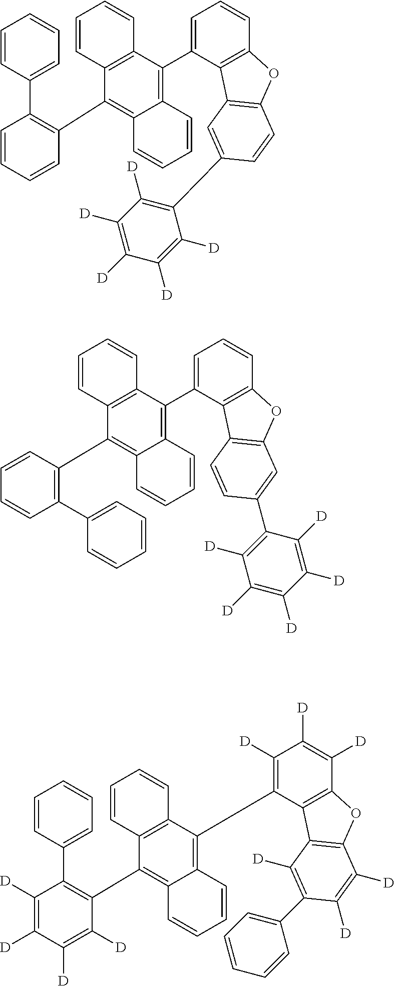 [SMILES: [2H]c1c([2H])c([2H])c(-c2ccc3oc4cccc(-c5c6ccccc6c(-c6ccccc6-c6ccccc6)c6ccccc56)c4c3c2)c([2H])c1[2H] ; [2H]c1c([2H])c([2H])c(-c2ccc3c(c2)oc2cccc(-c4c5ccccc5c(-c5ccccc5-c5ccccc5)c5ccccc45)c23)c([2H])c1[2H] ; [2H]c1c([2H])c([2H])c(-c2c3ccccc3c(-c3c([2H])c([2H])c([2H])c4oc5c([2H])c([2H])c(-c6ccccc6)c([2H])c5c34)c3ccccc23)c(-c2ccccc2)c1[2H]]

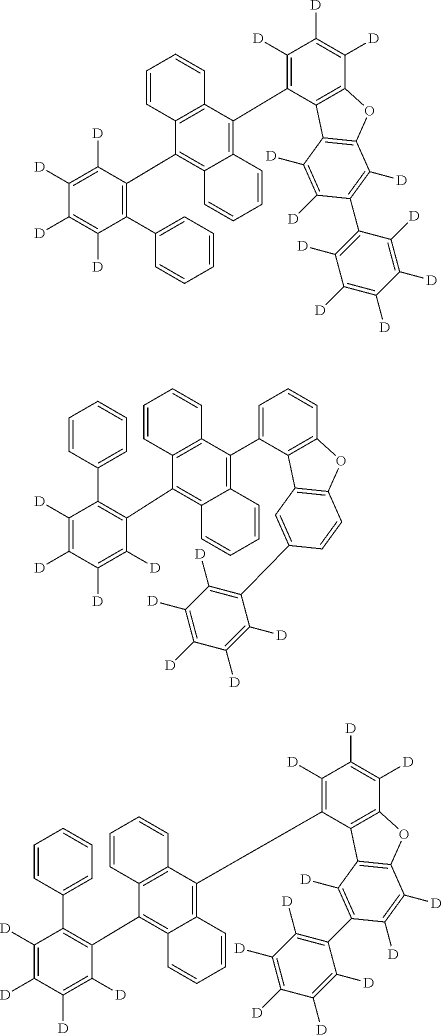 [SMILES: [2H]c1c([2H])c([2H])c(-c2c([2H])c([2H])c3c(oc4c([2H])c([2H])c([2H])c(-c5c6ccccc6c(-c6c([2H])c([2H])c([2H])c([2H])c6-c6ccccc6)c6ccccc56)c43)c2[2H])c([2H])c1[2H] ; [2H]c1c([2H])c([2H])c(-c2ccc3oc4cccc(-c5c6ccccc6c(-c6c([2H])c([2H])c([2H])c([2H])c6-c6ccccc6)c6ccccc56)c4c3c2)c([2H])c1[2H] ; [2H]c1c([2H])c([2H])c(-c2c([2H])c([2H])c3oc4c([2H])c([2H])c([2H])c(-c5c6ccccc6c(-c6c([2H])c([2H])c([2H])c([2H])c6-c6ccccc6)c6ccccc56)c4c3c2[2H])c([2H])c1[2H]]

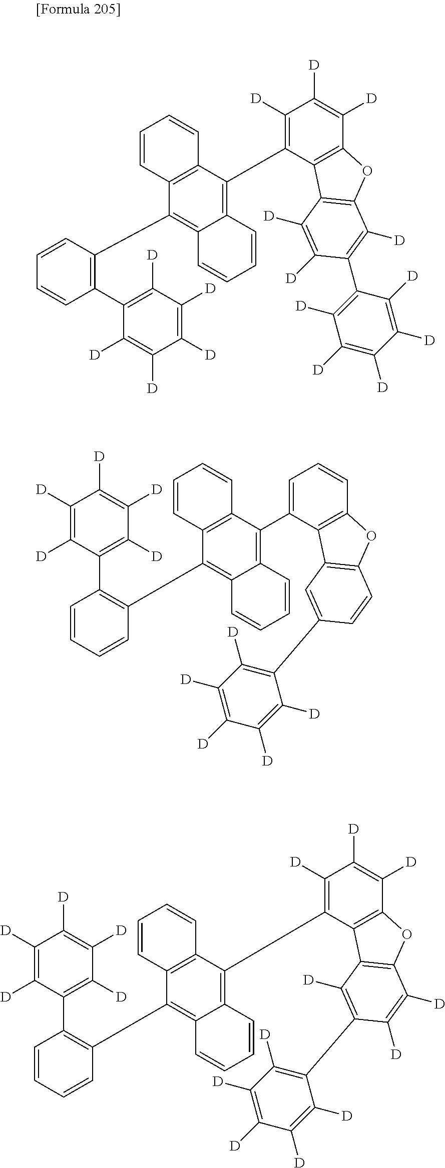 [SMILES: [2H]c1c([2H])c([2H])c(-c2ccccc2-c2c3ccccc3c(-c3c([2H])c([2H])c([2H])c4oc5c([2H])c(-c6c([2H])c([2H])c([2H])c([2H])c6[2H])c([2H])c([2H])c5c34)c3ccccc23)c([2H])c1[2H] ; [2H]c1c([2H])c([2H])c(-c2ccc3oc4cccc(-c5c6ccccc6c(-c6ccccc6-c6c([2H])c([2H])c([2H])c([2H])c6[2H])c6ccccc56)c4c3c2)c([2H])c1[2H] ; [2H]c1c([2H])c([2H])c(-c2ccccc2-c2c3ccccc3c(-c3c([2H])c([2H])c([2H])c4oc5c([2H])c([2H])c(-c6c([2H])c([2H])c([2H])c([2H])c6[2H])c([2H])c5c34)c3ccccc23)c([2H])c1[2H]]

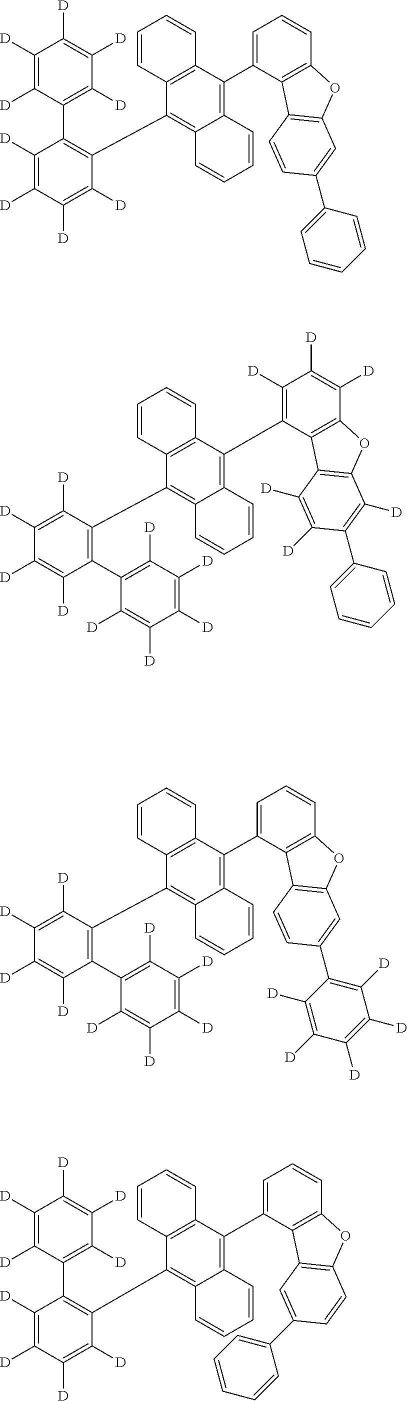

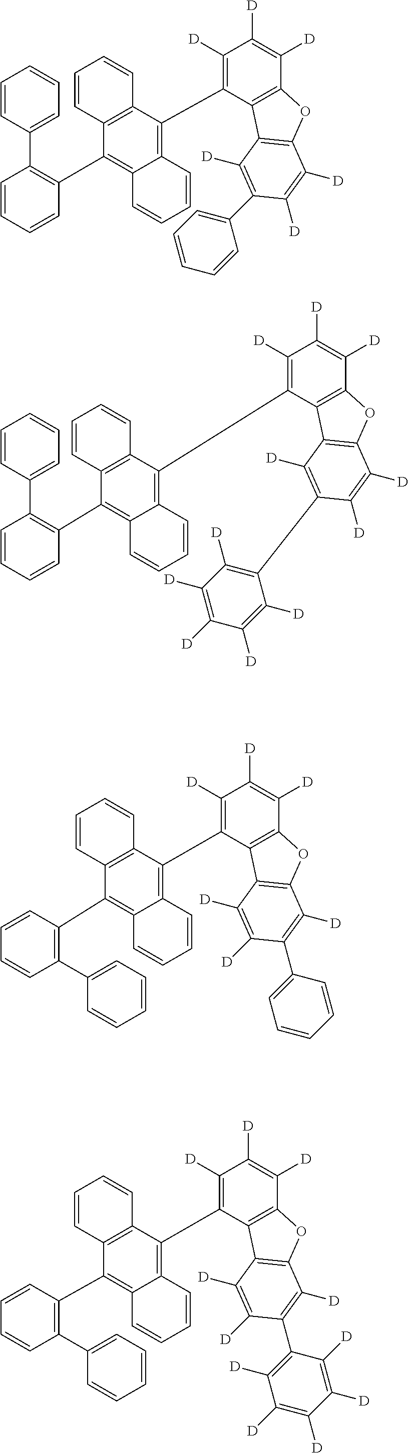 [SMILES: [2H]c1c(-c2ccccc2)c([2H])c2c(oc3c([2H])c([2H])c([2H])c(-c4c5ccccc5c(-c5ccccc5-c5ccccc5)c5ccccc45)c32)c1[2H] ; [2H]c1c([2H])c([2H])c(-c2c([2H])c([2H])c3oc4c([2H])c([2H])c([2H])c(-c5c6ccccc6c(-c6ccccc6-c6ccccc6)c6ccccc56)c4c3c2[2H])c([2H])c1[2H] ; [2H]c1c([2H])c(-c2c3ccccc3c(-c3ccccc3-c3ccccc3)c3ccccc23)c2c(oc3c([2H])c(-c4ccccc4)c([2H])c([2H])c32)c1[2H] ; [2H]c1c([2H])c([2H])c(-c2c([2H])c([2H])c3c(oc4c([2H])c([2H])c([2H])c(-c5c6ccccc6c(-c6ccccc6-c6ccccc6)c6ccccc56)c43)c2[2H])c([2H])c1[2H]]

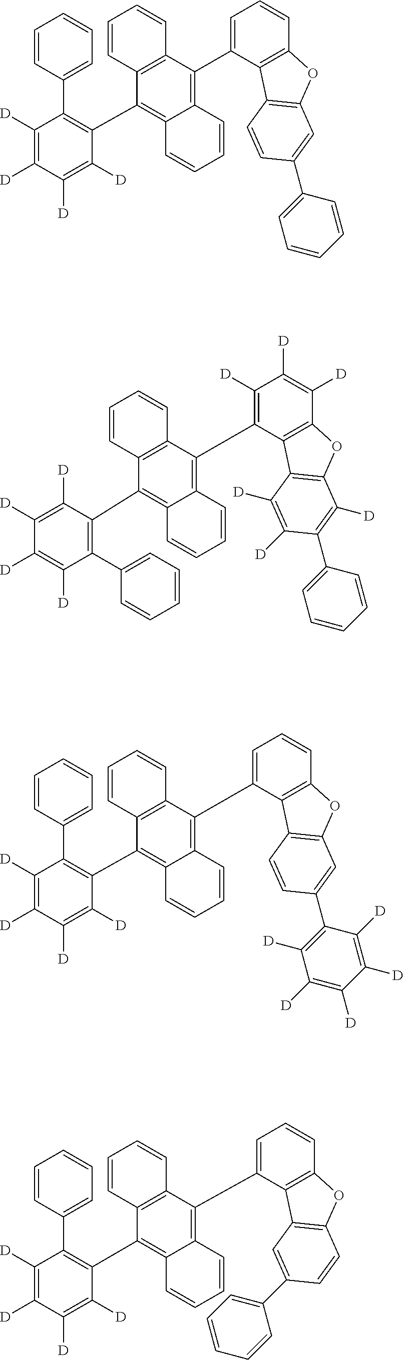 [SMILES: [2H]c1c([2H])c([2H])c(-c2c3ccccc3c(-c3cccc4oc5cc(-c6ccccc6)ccc5c34)c3ccccc23)c(-c2ccccc2)c1[2H] ; [2H]c1c([2H])c([2H])c(-c2c3ccccc3c(-c3c([2H])c([2H])c([2H])c4oc5c([2H])c(-c6ccccc6)c([2H])c([2H])c5c34)c3ccccc23)c(-c2ccccc2)c1[2H] ; [2H]c1c([2H])c([2H])c(-c2ccc3c(c2)oc2cccc(-c4c5ccccc5c(-c5c([2H])c([2H])c([2H])c([2H])c5-c5ccccc5)c5ccccc45)c23)c([2H])c1[2H] ; [2H]c1c([2H])c([2H])c(-c2c3ccccc3c(-c3cccc4oc5ccc(-c6ccccc6)cc5c34)c3ccccc23)c(-c2ccccc2)c1[2H]]

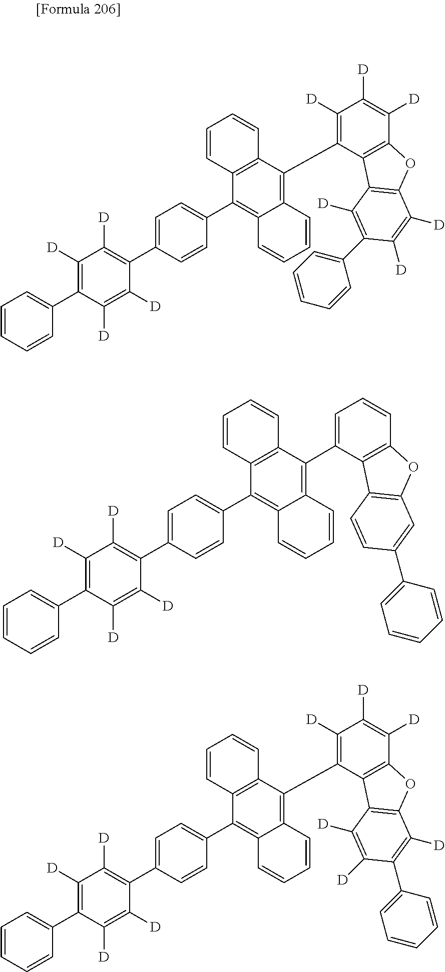 [SMILES: [2H]c1c([2H])c(-c2ccc(-c3c4ccccc4c(-c4c([2H])c([2H])c([2H])c5oc6c([2H])c([2H])c(-c7ccccc7)c([2H])c6c45)c4ccccc34)cc2)c([2H])c([2H])c1-c1ccccc1 ; [2H]c1c([2H])c(-c2ccc(-c3c4ccccc4c(-c4cccc5oc6cc(-c7ccccc7)ccc6c45)c4ccccc34)cc2)c([2H])c([2H])c1-c1ccccc1 ; [2H]c1c([2H])c(-c2ccc(-c3c4ccccc4c(-c4c([2H])c([2H])c([2H])c5oc6c([2H])c(-c7ccccc7)c([2H])c([2H])c6c45)c4ccccc34)cc2)c([2H])c([2H])c1-c1ccccc1]

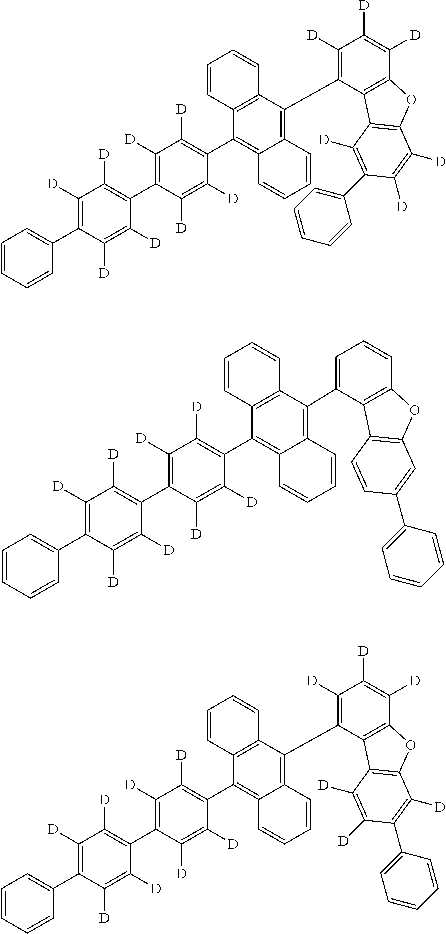 [SMILES: [2H]c1c([2H])c(-c2c([2H])c([2H])c(-c3c4ccccc4c(-c4c([2H])c([2H])c([2H])c5oc6c([2H])c([2H])c(-c7ccccc7)c([2H])c6c45)c4ccccc34)c([2H])c2[2H])c([2H])c([2H])c1-c1ccccc1 ; [2H]c1c([2H])c(-c2c([2H])c([2H])c(-c3c4ccccc4c(-c4cccc5oc6cc(-c7ccccc7)ccc6c45)c4ccccc34)c([2H])c2[2H])c([2H])c([2H])c1-c1ccccc1 ; [2H]c1c([2H])c(-c2c([2H])c([2H])c(-c3c4ccccc4c(-c4c([2H])c([2H])c([2H])c5oc6c([2H])c(-c7ccccc7)c([2H])c([2H])c6c45)c4ccccc34)c([2H])c2[2H])c([2H])c([2H])c1-c1ccccc1]

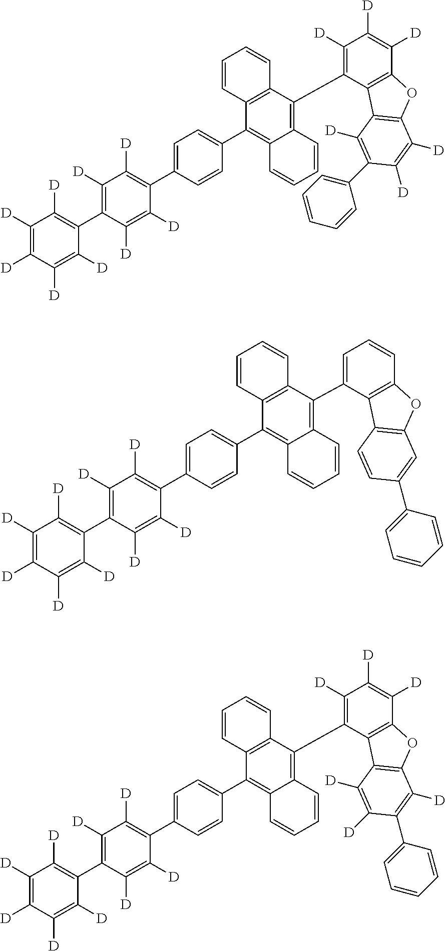 [SMILES: [2H]c1c([2H])c([2H])c(-c2c([2H])c([2H])c(-c3ccc(-c4c5ccccc5c(-c5c([2H])c([2H])c([2H])c6oc7c([2H])c([2H])c(-c8ccccc8)c([2H])c7c56)c5ccccc45)cc3)c([2H])c2[2H])c([2H])c1[2H] ; [2H]c1c([2H])c([2H])c(-c2c([2H])c([2H])c(-c3ccc(-c4c5ccccc5c(-c5cccc6oc7cc(-c8ccccc8)ccc7c56)c5ccccc45)cc3)c([2H])c2[2H])c([2H])c1[2H] ; [2H]c1c([2H])c([2H])c(-c2c([2H])c([2H])c(-c3ccc(-c4c5ccccc5c(-c5c([2H])c([2H])c([2H])c6oc7c([2H])c(-c8ccccc8)c([2H])c([2H])c7c56)c5ccccc45)cc3)c([2H])c2[2H])c([2H])c1[2H]]

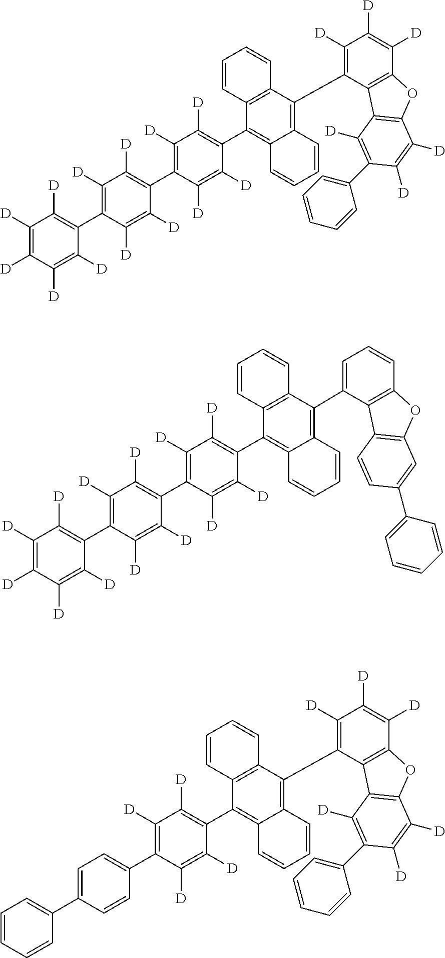 [SMILES: [2H]c1c([2H])c([2H])c(-c2c([2H])c([2H])c(-c3c([2H])c([2H])c(-c4c5ccccc5c(-c5c([2H])c([2H])c([2H])c6oc7c([2H])c([2H])c(-c8ccccc8)c([2H])c7c56)c5ccccc45)c([2H])c3[2H])c([2H])c2[2H])c([2H])c1[2H] ; [2H]c1c([2H])c([2H])c(-c2c([2H])c([2H])c(-c3c([2H])c([2H])c(-c4c5ccccc5c(-c5cccc6oc7cc(-c8ccccc8)ccc7c56)c5ccccc45)c([2H])c3[2H])c([2H])c2[2H])c([2H])c1[2H] ; [2H]c1c([2H])c(-c2c3ccccc3c(-c3c([2H])c([2H])c([2H])c4oc5c([2H])c([2H])c(-c6ccccc6)c([2H])c5c34)c3ccccc23)c([2H])c([2H])c1-c1ccc(-c2ccccc2)cc1]

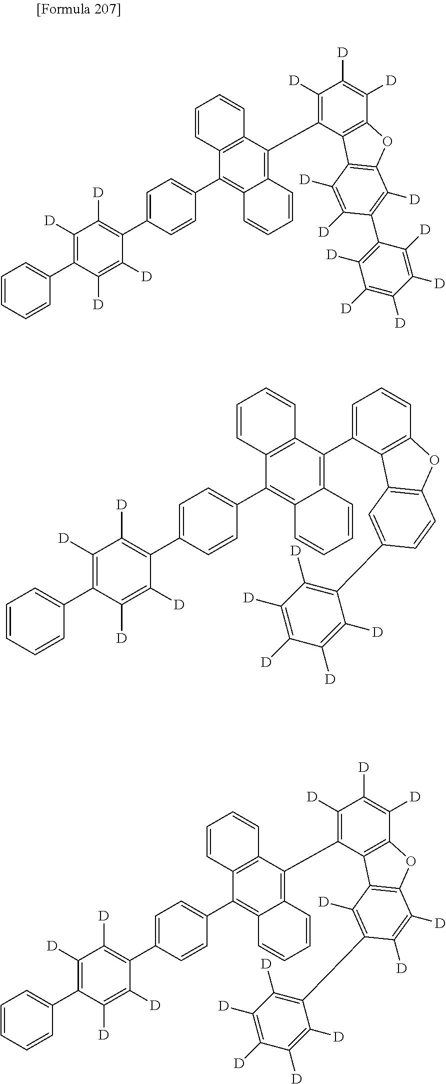 [SMILES: [2H]c1c([2H])c([2H])c(-c2c([2H])c([2H])c3c(oc4c([2H])c([2H])c([2H])c(-c5c6ccccc6c(-c6ccc(-c7c([2H])c([2H])c(-c8ccccc8)c([2H])c7[2H])cc6)c6ccccc56)c43)c2[2H])c([2H])c1[2H] ; [2H]c1c([2H])c([2H])c(-c2ccc3oc4cccc(-c5c6ccccc6c(-c6ccc(-c7c([2H])c([2H])c(-c8ccccc8)c([2H])c7[2H])cc6)c6ccccc56)c4c3c2)c([2H])c1[2H] ; [2H]c1c([2H])c([2H])c(-c2c([2H])c([2H])c3oc4c([2H])c([2H])c([2H])c(-c5c6ccccc6c(-c6ccc(-c7c([2H])c([2H])c(-c8ccccc8)c([2H])c7[2H])cc6)c6ccccc56)c4c3c2[2H])c([2H])c1[2H]]

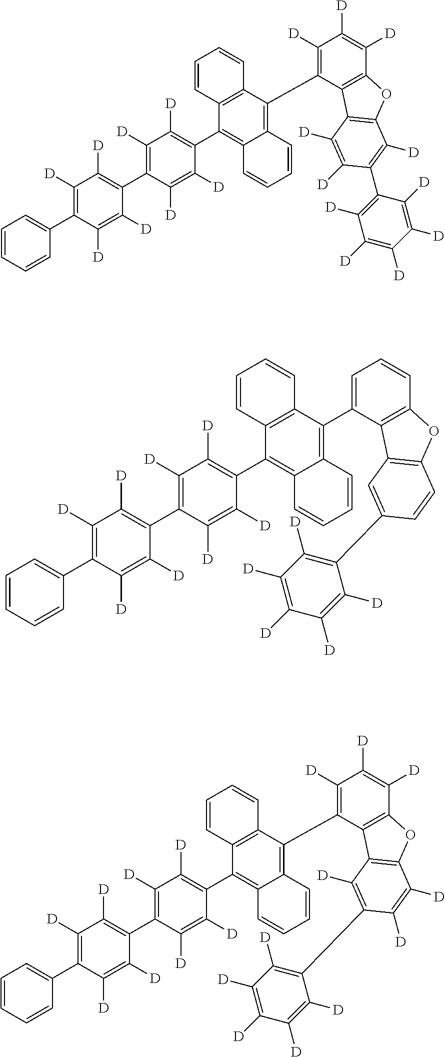

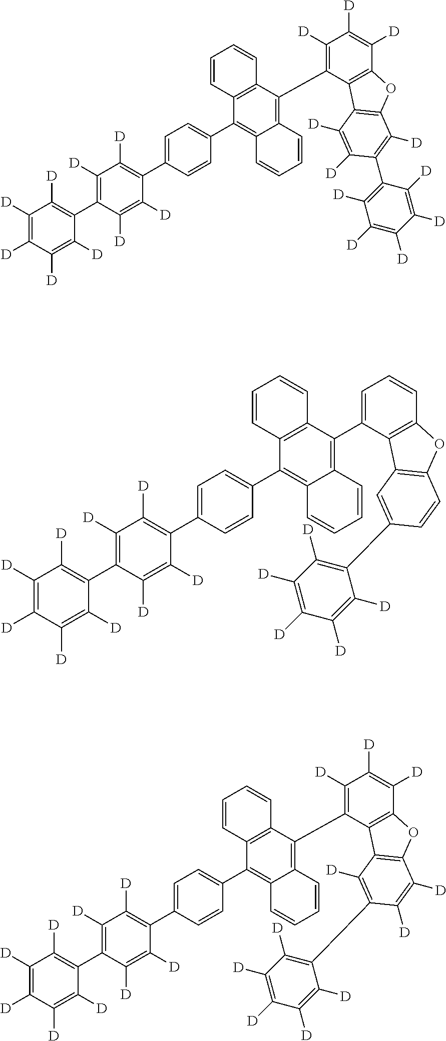

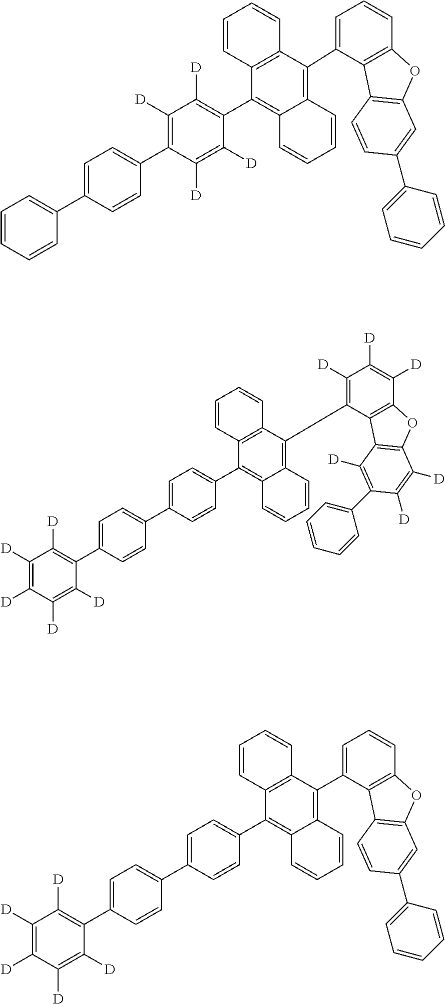 [SMILES: [2H]c1c([2H])c(-c2c3ccccc3c(-c3cccc4oc5cc(-c6ccccc6)ccc5c34)c3ccccc23)c([2H])c([2H])c1-c1ccc(-c2ccccc2)cc1 ; [2H]c1c([2H])c([2H])c(-c2ccc(-c3ccc(-c4c5ccccc5c(-c5c([2H])c([2H])c([2H])c6oc7c([2H])c([2H])c(-c8ccccc8)c([2H])c7c56)c5ccccc45)cc3)cc2)c([2H])c1[2H] ; [2H]c1c([2H])c([2H])c(-c2ccc(-c3ccc(-c4c5ccccc5c(-c5cccc6oc7cc(-c8ccccc8)ccc7c56)c5ccccc45)cc3)cc2)c([2H])c1[2H]]

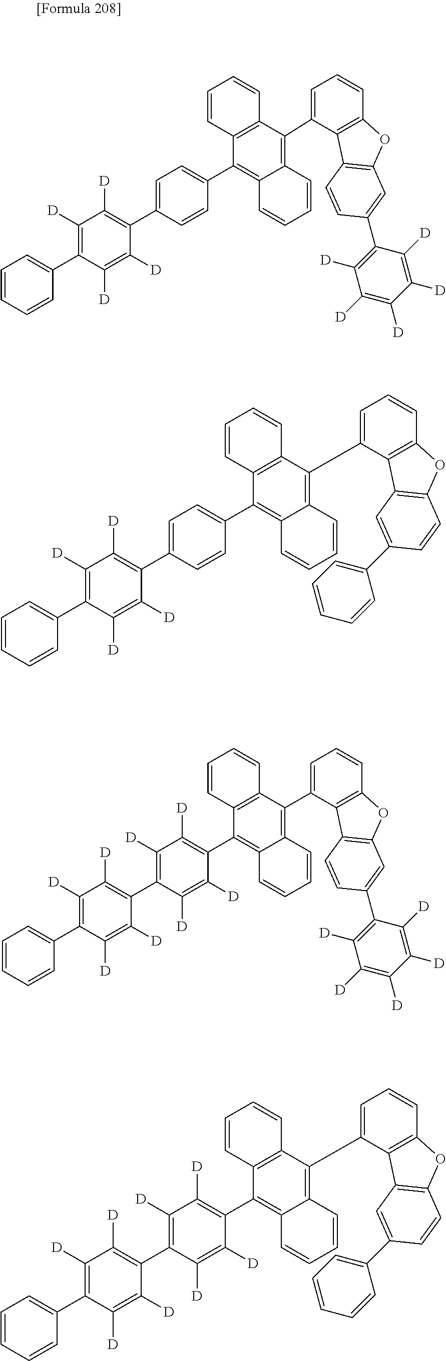

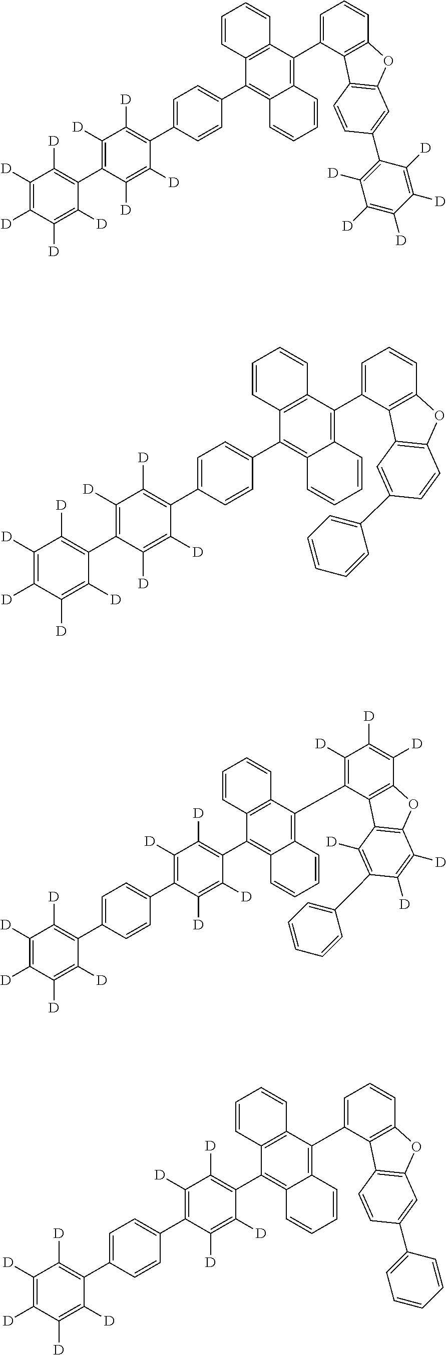

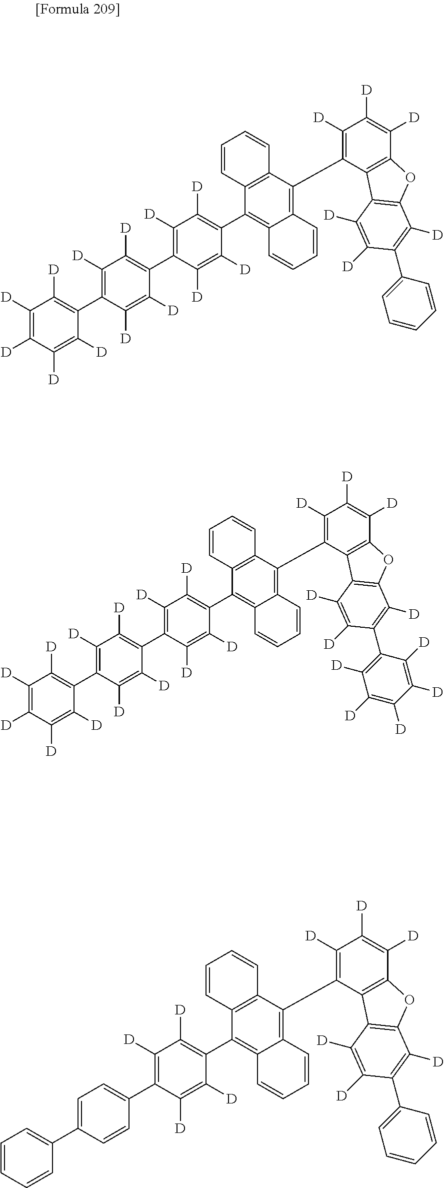

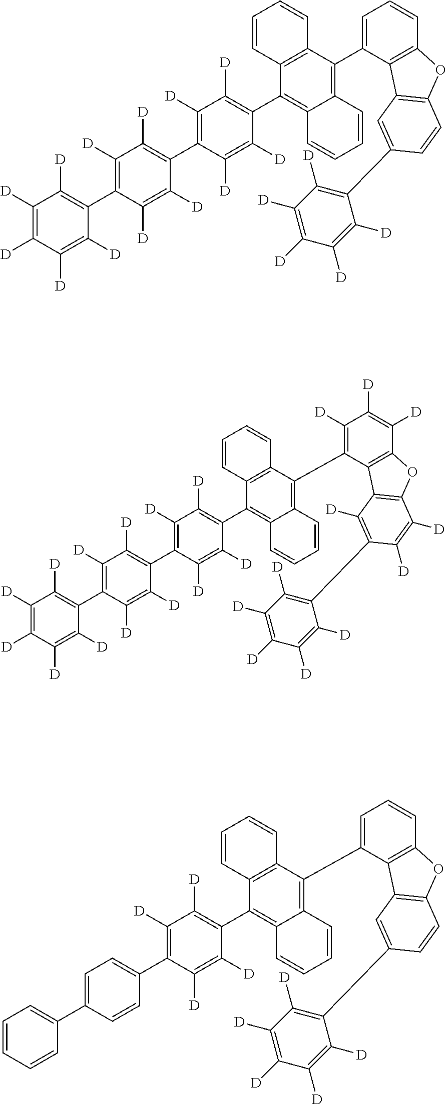

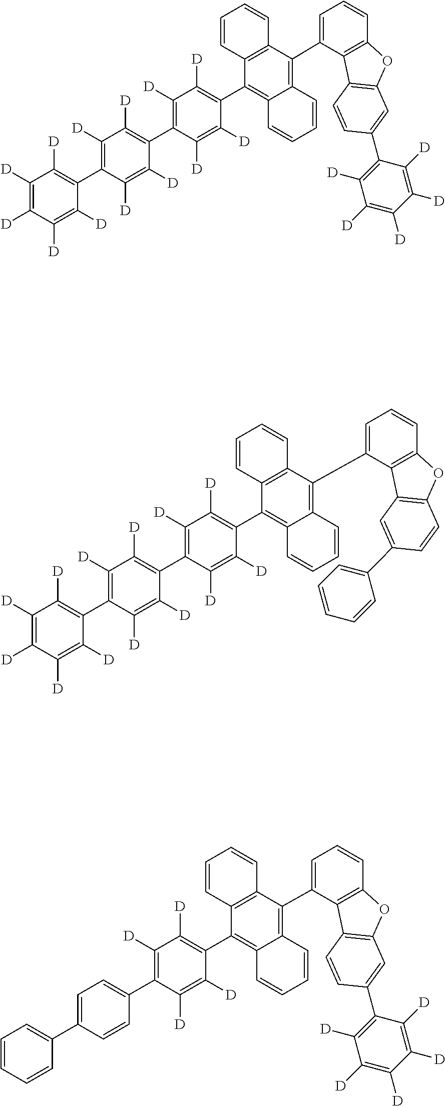 [SMILES: [2H]c1c([2H])c([2H])c(-c2ccc3c(c2)oc2cccc(-c4c5ccccc5c(-c5c([2H])c([2H])c(-c6c([2H])c([2H])c(-c7c([2H])c([2H])c([2H])c([2H])c7[2H])c([2H])c6[2H])c([2H])c5[2H])c5ccccc45)c23)c([2H])c1[2H] ; [2H]c1c([2H])c([2H])c(-c2c([2H])c([2H])c(-c3c([2H])c([2H])c(-c4c5ccccc5c(-c5cccc6oc7ccc(-c8ccccc8)cc7c56)c5ccccc45)c([2H])c3[2H])c([2H])c2[2H])c([2H])c1[2H] ; [2H]c1c([2H])c([2H])c(-c2ccc3c(c2)oc2cccc(-c4c5ccccc5c(-c5c([2H])c([2H])c(-c6ccc(-c7ccccc7)cc6)c([2H])c5[2H])c5ccccc45)c23)c([2H])c1[2H]]

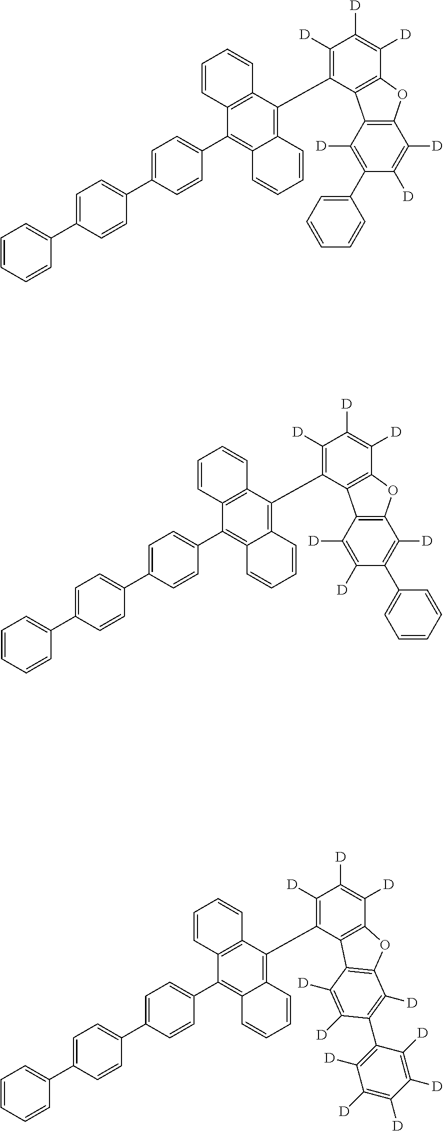 [SMILES: [2H]c1c(-c2ccccc2)c([2H])c2c(oc3c([2H])c([2H])c([2H])c(-c4c5ccccc5c(-c5ccc(-c6ccc(-c7ccccc7)cc6)cc5)c5ccccc45)c32)c1[2H] ; [2H]c1c([2H])c(-c2c3ccccc3c(-c3ccc(-c4ccc(-c5ccccc5)cc4)cc3)c3ccccc23)c2c(oc3c([2H])c(-c4ccccc4)c([2H])c([2H])c32)c1[2H] ; [2H]c1c([2H])c([2H])c(-c2c([2H])c([2H])c3c(oc4c([2H])c([2H])c([2H])c(-c5c6ccccc6c(-c6ccc(-c7ccc(-c8ccccc8)cc7)cc6)c6ccccc56)c43)c2[2H])c([2H])c1[2H]]

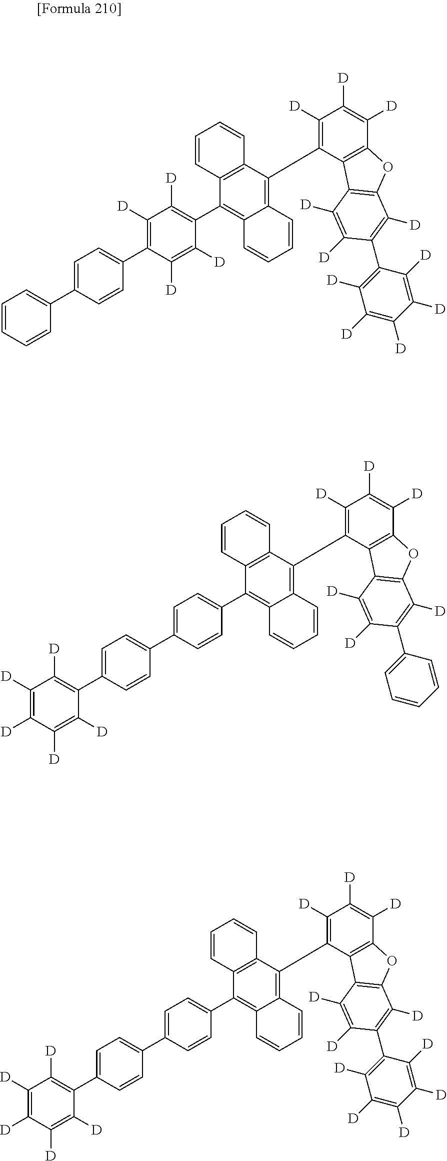 [SMILES: [2H]c1c([2H])c([2H])c(-c2c([2H])c([2H])c3c(oc4c([2H])c([2H])c([2H])c(-c5c6ccccc6c(-c6c([2H])c([2H])c(-c7ccc(-c8ccccc8)cc7)c([2H])c6[2H])c6ccccc56)c43)c2[2H])c([2H])c1[2H] ; [2H]c1c([2H])c([2H])c(-c2ccc(-c3ccc(-c4c5ccccc5c(-c5c([2H])c([2H])c([2H])c6oc7c([2H])c(-c8ccccc8)c([2H])c([2H])c7c56)c5ccccc45)cc3)cc2)c([2H])c1[2H] ; [2H]c1c([2H])c([2H])c(-c2ccc(-c3ccc(-c4c5ccccc5c(-c5c([2H])c([2H])c([2H])c6oc7c([2H])c(-c8c([2H])c([2H])c([2H])c([2H])c8[2H])c([2H])c([2H])c7c56)c5ccccc45)cc3)cc2)c([2H])c1[2H]]

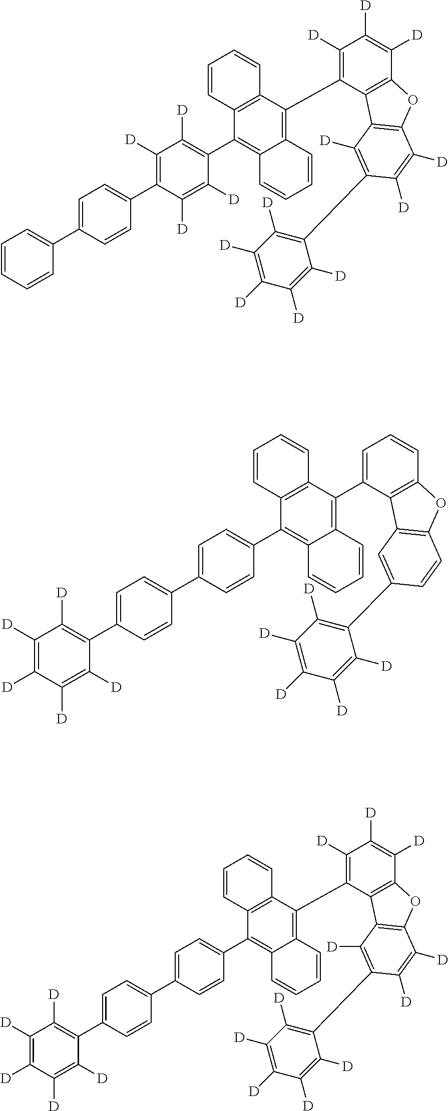 [SMILES: [2H]c1c([2H])c([2H])c(-c2c([2H])c([2H])c3oc4c([2H])c([2H])c([2H])c(-c5c6ccccc6c(-c6c([2H])c([2H])c(-c7ccc(-c8ccccc8)cc7)c([2H])c6[2H])c6ccccc56)c4c3c2[2H])c([2H])c1[2H] ; [2H]c1c([2H])c([2H])c(-c2ccc(-c3ccc(-c4c5ccccc5c(-c5cccc6oc7ccc(-c8c([2H])c([2H])c([2H])c([2H])c8[2H])cc7c56)c5ccccc45)cc3)cc2)c([2H])c1[2H] ; [2H]c1c([2H])c([2H])c(-c2ccc(-c3ccc(-c4c5ccccc5c(-c5c([2H])c([2H])c([2H])c6oc7c([2H])c([2H])c(-c8c([2H])c([2H])c([2H])c([2H])c8[2H])c([2H])c7c56)c5ccccc45)cc3)cc2)c([2H])c1[2H]]

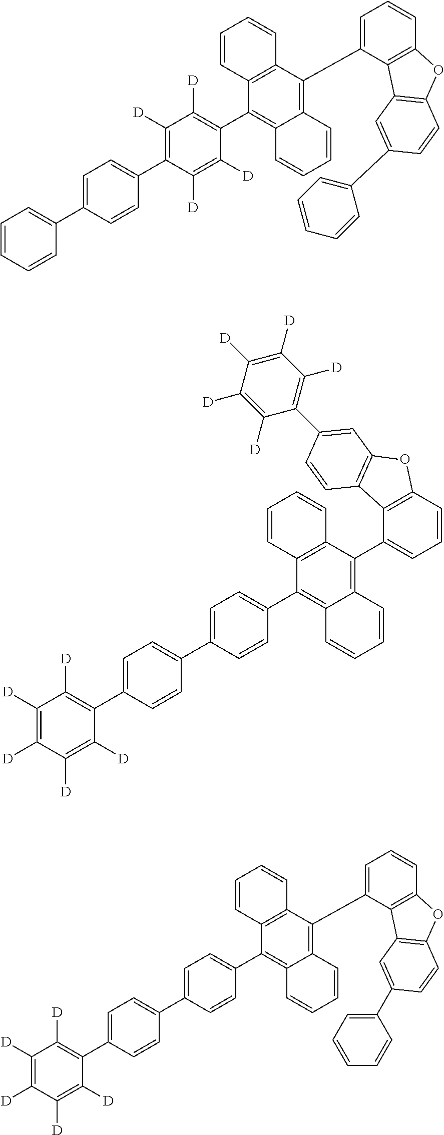 [SMILES: [2H]c1c([2H])c(-c2c3ccccc3c(-c3cccc4oc5ccc(-c6ccccc6)cc5c34)c3ccccc23)c([2H])c([2H])c1-c1ccc(-c2ccccc2)cc1 ; [2H]c1c([2H])c([2H])c(-c2ccc(-c3ccc(-c4c5ccccc5c(-c5cccc6oc7cc(-c8c([2H])c([2H])c([2H])c([2H])c8[2H])ccc7c56)c5ccccc45)cc3)cc2)c([2H])c1[2H] ; [2H]c1c([2H])c([2H])c(-c2ccc(-c3ccc(-c4c5ccccc5c(-c5cccc6oc7ccc(-c8ccccc8)cc7c56)c5ccccc45)cc3)cc2)c([2H])c1[2H]]

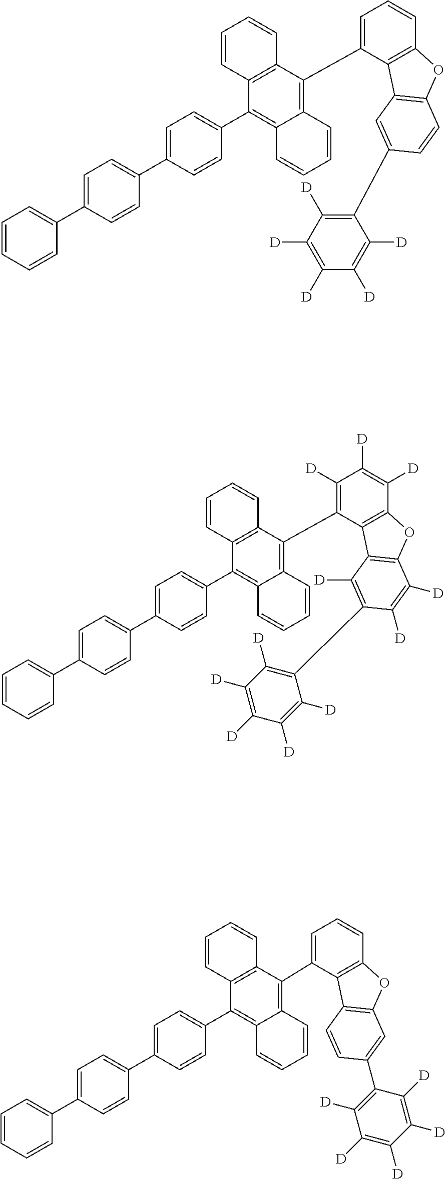 [SMILES: [2H]c1c([2H])c([2H])c(-c2ccc3oc4cccc(-c5c6ccccc6c(-c6ccc(-c7ccc(-c8ccccc8)cc7)cc6)c6ccccc56)c4c3c2)c([2H])c1[2H] ; [2H]c1c([2H])c([2H])c(-c2c([2H])c([2H])c3oc4c([2H])c([2H])c([2H])c(-c5c6ccccc6c(-c6ccc(-c7ccc(-c8ccccc8)cc7)cc6)c6ccccc56)c4c3c2[2H])c([2H])c1[2H] ; [2H]c1c([2H])c([2H])c(-c2ccc3c(c2)oc2cccc(-c4c5ccccc5c(-c5ccc(-c6ccc(-c7ccccc7)cc6)cc5)c5ccccc45)c23)c([2H])c1[2H]]

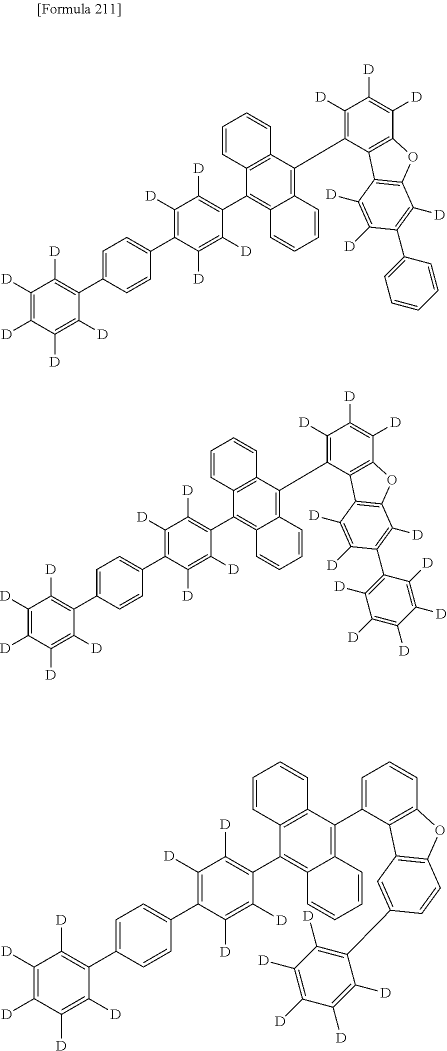

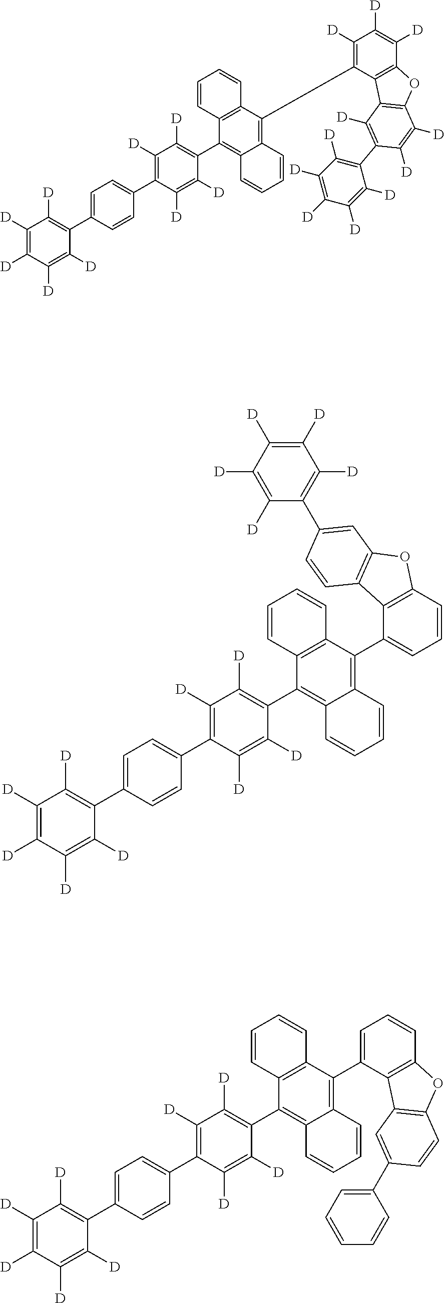 [SMILES: [2H]c1c([2H])c([2H])c(-c2ccc(-c3c([2H])c([2H])c(-c4c5ccccc5c(-c5c([2H])c([2H])c([2H])c6oc7c([2H])c([2H])c(-c8c([2H])c([2H])c([2H])c([2H])c8[2H])c([2H])c7c56)c5ccccc45)c([2H])c3[2H])cc2)c([2H])c1[2H] ; [2H]c1c([2H])c([2H])c(-c2ccc(-c3c([2H])c([2H])c(-c4c5ccccc5c(-c5cccc6oc7cc(-c8c([2H])c([2H])c([2H])c([2H])c8[2H])ccc7c56)c5ccccc45)c([2H])c3[2H])cc2)c([2H])c1[2H] ; [2H]c1c([2H])c([2H])c(-c2ccc(-c3c([2H])c([2H])c(-c4c5ccccc5c(-c5cccc6oc7ccc(-c8ccccc8)cc7c56)c5ccccc45)c([2H])c3[2H])cc2)c([2H])c1[2H]]

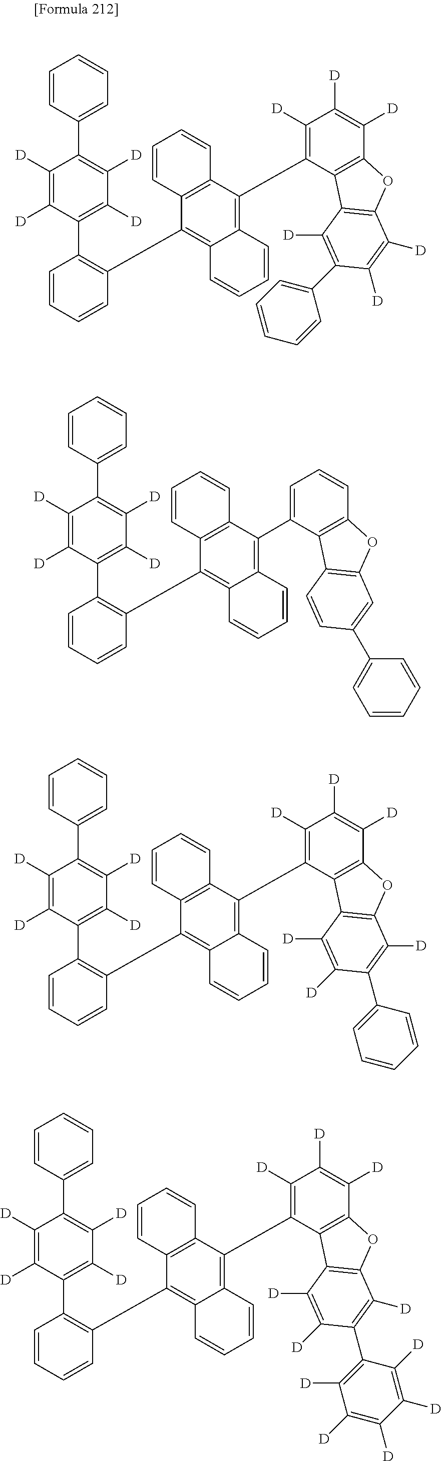

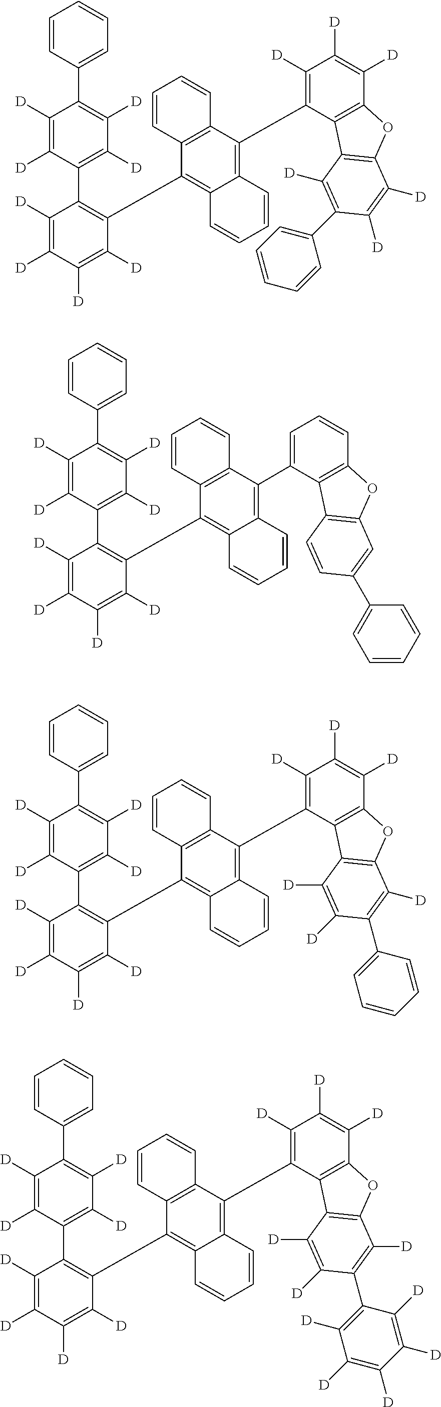

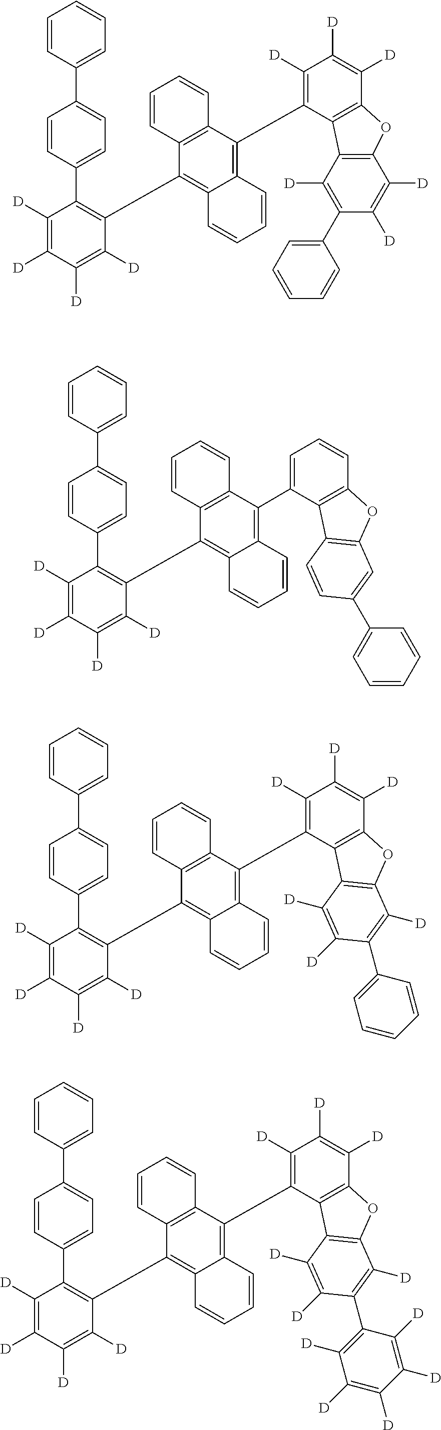

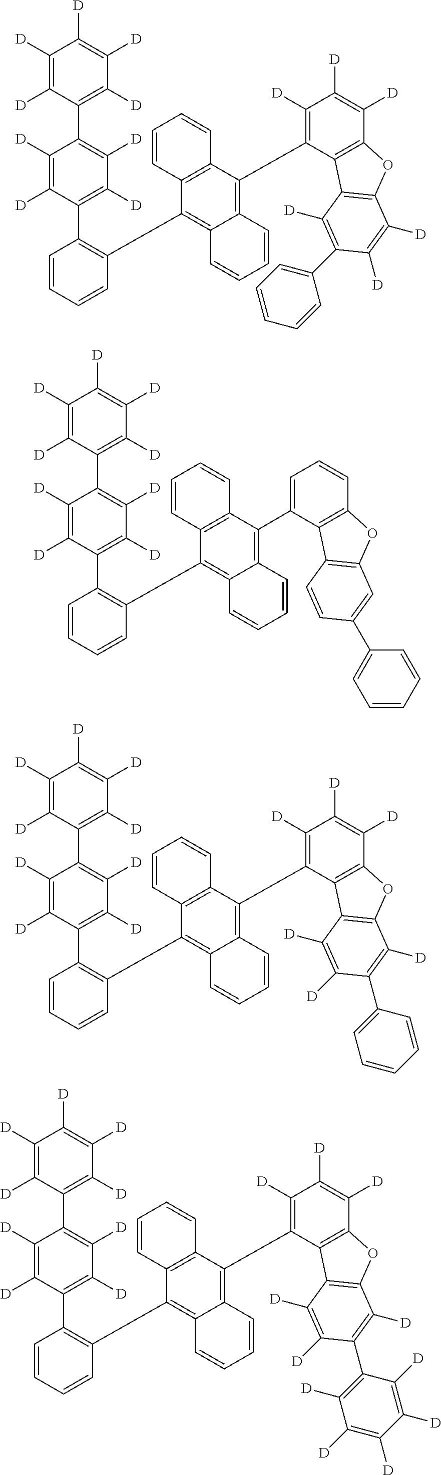

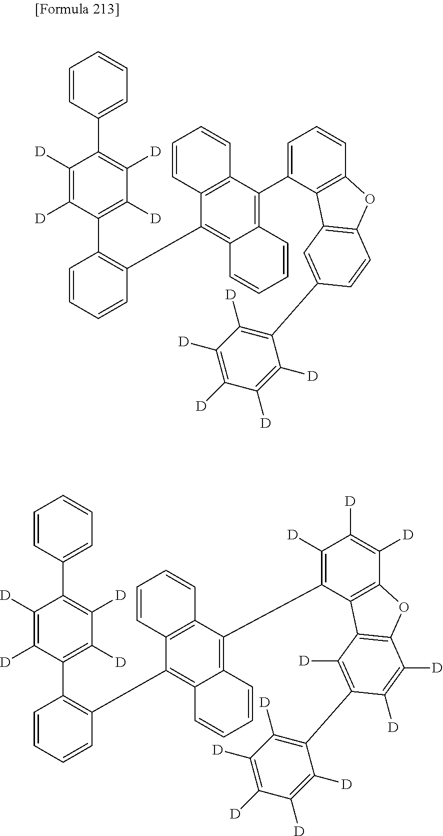 [SMILES: [2H]c1c([2H])c([2H])c(-c2ccc3oc4cccc(-c5c6ccccc6c(-c6ccccc6-c6c([2H])c([2H])c(-c7ccccc7)c([2H])c6[2H])c6ccccc56)c4c3c2)c([2H])c1[2H] ; [2H]c1c([2H])c([2H])c(-c2c([2H])c([2H])c3oc4c([2H])c([2H])c([2H])c(-c5c6ccccc6c(-c6ccccc6-c6c([2H])c([2H])c(-c7ccccc7)c([2H])c6[2H])c6ccccc56)c4c3c2[2H])c([2H])c1[2H]]

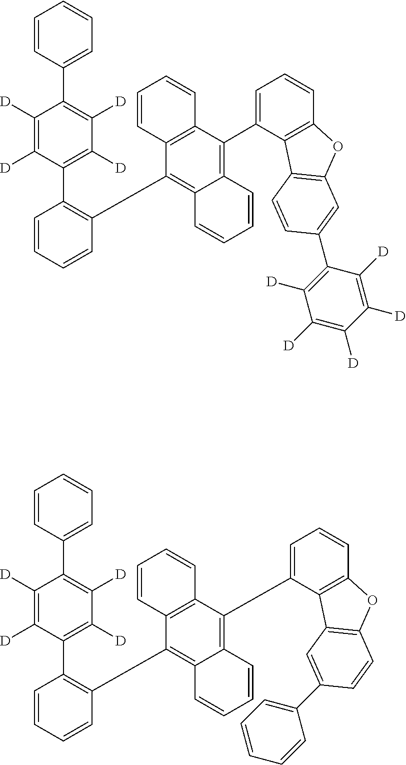 [SMILES: [2H]c1c([2H])c([2H])c(-c2ccc3c(c2)oc2cccc(-c4c5ccccc5c(-c5ccccc5-c5c([2H])c([2H])c(-c6ccccc6)c([2H])c5[2H])c5ccccc45)c23)c([2H])c1[2H] ; [2H]c1c([2H])c(-c2ccccc2-c2c3ccccc3c(-c3cccc4oc5ccc(-c6ccccc6)cc5c34)c3ccccc23)c([2H])c([2H])c1-c1ccccc1]

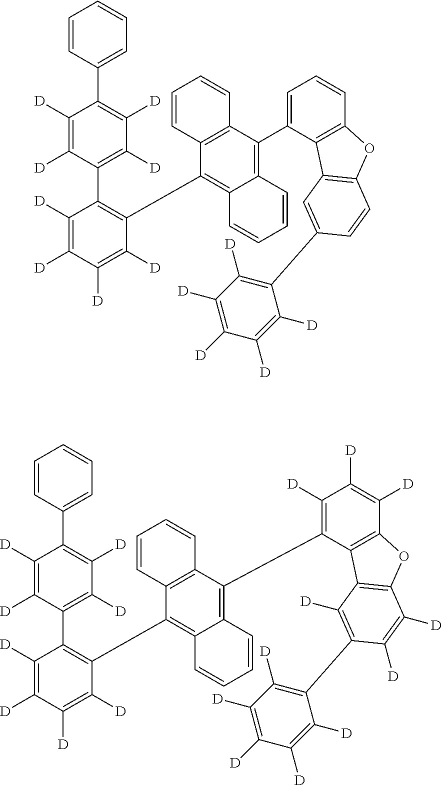 [SMILES: [2H]c1c([2H])c([2H])c(-c2ccc3oc4cccc(-c5c6ccccc6c(-c6c([2H])c([2H])c([2H])c([2H])c6-c6c([2H])c([2H])c(-c7ccccc7)c([2H])c6[2H])c6ccccc56)c4c3c2)c([2H])c1[2H] ; [2H]c1c([2H])c([2H])c(-c2c([2H])c([2H])c3oc4c([2H])c([2H])c([2H])c(-c5c6ccccc6c(-c6c([2H])c([2H])c([2H])c([2H])c6-c6c([2H])c([2H])c(-c7ccccc7)c([2H])c6[2H])c6ccccc56)c4c3c2[2H])c([2H])c1[2H]]

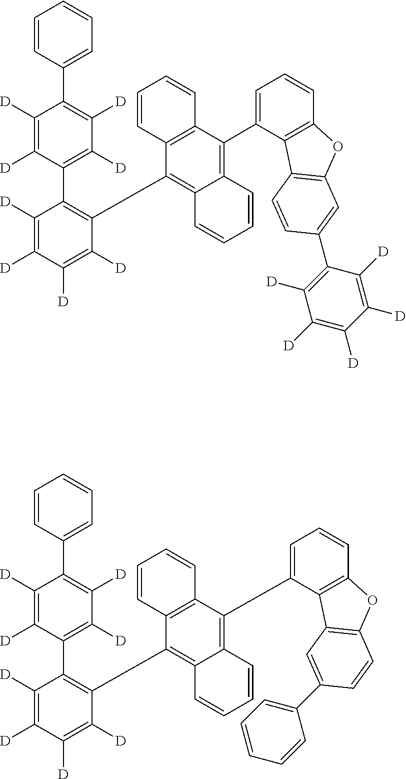 [SMILES: [2H]c1c([2H])c([2H])c(-c2ccc3c(c2)oc2cccc(-c4c5ccccc5c(-c5c([2H])c([2H])c([2H])c([2H])c5-c5c([2H])c([2H])c(-c6ccccc6)c([2H])c5[2H])c5ccccc45)c23)c([2H])c1[2H] ; [2H]c1c([2H])c([2H])c(-c2c3ccccc3c(-c3cccc4oc5ccc(-c6ccccc6)cc5c34)c3ccccc23)c(-c2c([2H])c([2H])c(-c3ccccc3)c([2H])c2[2H])c1[2H]]

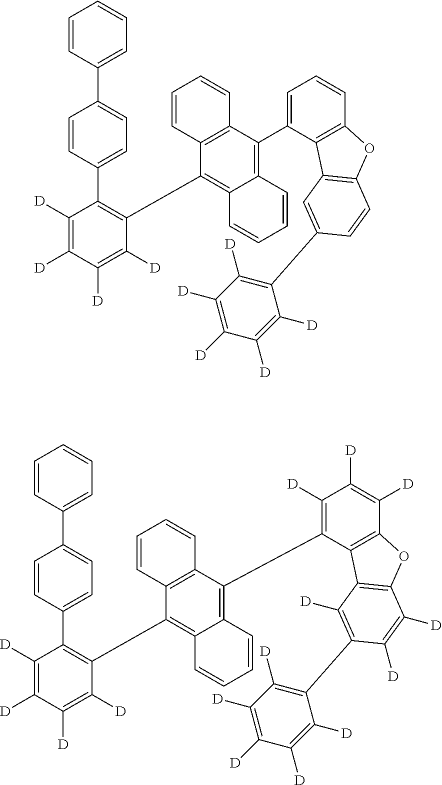 [SMILES: [2H]c1c([2H])c([2H])c(-c2ccc3oc4cccc(-c5c6ccccc6c(-c6c([2H])c([2H])c([2H])c([2H])c6-c6ccc(-c7ccccc7)cc6)c6ccccc56)c4c3c2)c([2H])c1[2H] ; [2H]c1c([2H])c([2H])c(-c2c([2H])c([2H])c3oc4c([2H])c([2H])c([2H])c(-c5c6ccccc6c(-c6c([2H])c([2H])c([2H])c([2H])c6-c6ccc(-c7ccccc7)cc6)c6ccccc56)c4c3c2[2H])c([2H])c1[2H]]

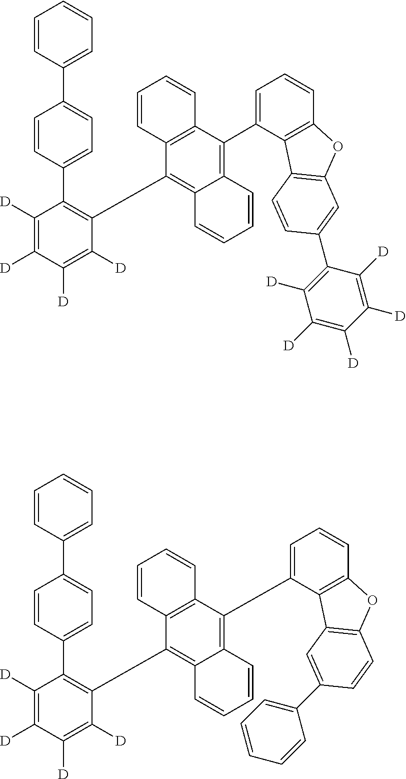 [SMILES: [2H]c1c([2H])c([2H])c(-c2ccc3c(c2)oc2cccc(-c4c5ccccc5c(-c5c([2H])c([2H])c([2H])c([2H])c5-c5ccc(-c6ccccc6)cc5)c5ccccc45)c23)c([2H])c1[2H] ; [2H]c1c([2H])c([2H])c(-c2c3ccccc3c(-c3cccc4oc5ccc(-c6ccccc6)cc5c34)c3ccccc23)c(-c2ccc(-c3ccccc3)cc2)c1[2H]]

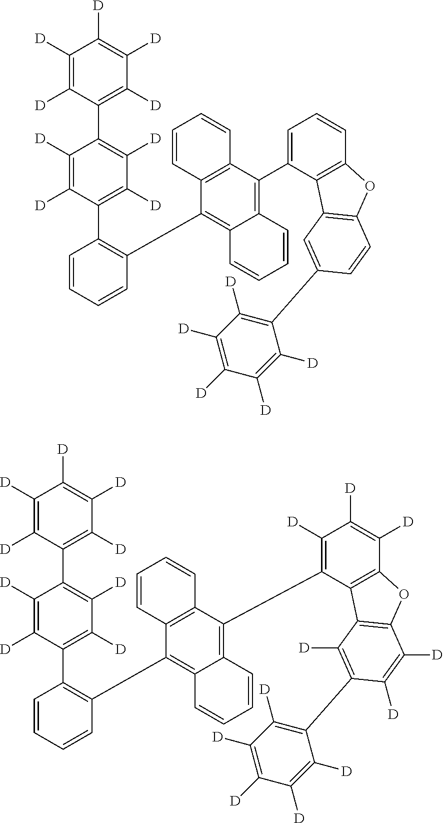 [SMILES: [2H]c1c([2H])c([2H])c(-c2ccc3oc4cccc(-c5c6ccccc6c(-c6ccccc6-c6c([2H])c([2H])c(-c7c([2H])c([2H])c([2H])c([2H])c7[2H])c([2H])c6[2H])c6ccccc56)c4c3c2)c([2H])c1[2H] ; [2H]c1c([2H])c([2H])c(-c2c([2H])c([2H])c(-c3ccccc3-c3c4ccccc4c(-c4c([2H])c([2H])c([2H])c5oc6c([2H])c([2H])c(-c7c([2H])c([2H])c([2H])c([2H])c7[2H])c([2H])c6c45)c4ccccc34)c([2H])c2[2H])c([2H])c1[2H]]

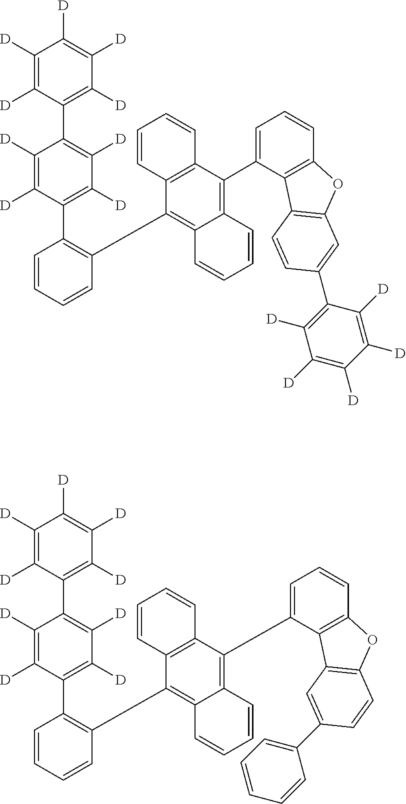 [SMILES: [2H]c1c([2H])c([2H])c(-c2ccc3c(c2)oc2cccc(-c4c5ccccc5c(-c5ccccc5-c5c([2H])c([2H])c(-c6c([2H])c([2H])c([2H])c([2H])c6[2H])c([2H])c5[2H])c5ccccc45)c23)c([2H])c1[2H] ; [2H]c1c([2H])c([2H])c(-c2c([2H])c([2H])c(-c3ccccc3-c3c4ccccc4c(-c4cccc5oc6ccc(-c7ccccc7)cc6c45)c4ccccc34)c([2H])c2[2H])c([2H])c1[2H]]

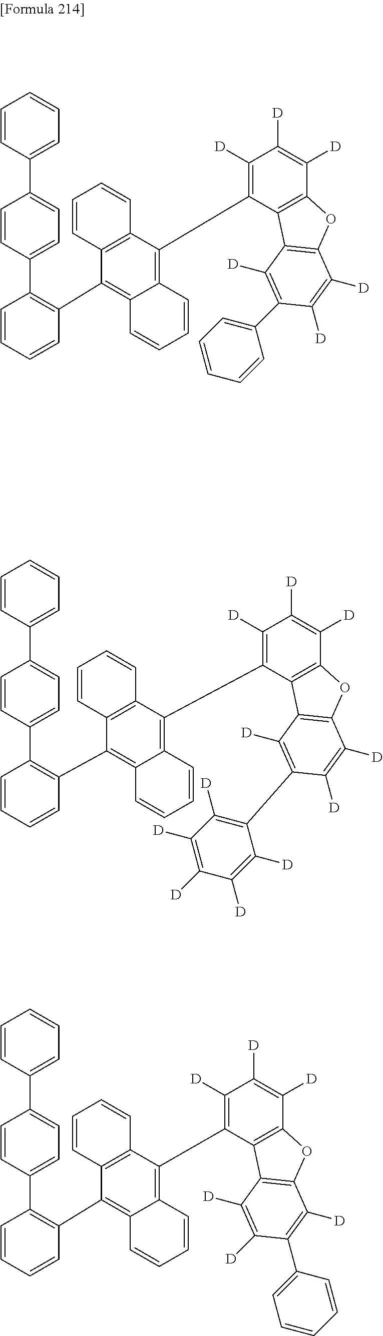 [SMILES: [2H]c1c(-c2ccccc2)c([2H])c2c(oc3c([2H])c([2H])c([2H])c(-c4c5ccccc5c(-c5ccccc5-c5ccc(-c6ccccc6)cc5)c5ccccc45)c32)c1[2H] ; [2H]c1c([2H])c([2H])c(-c2c([2H])c([2H])c3oc4c([2H])c([2H])c([2H])c(-c5c6ccccc6c(-c6ccccc6-c6ccc(-c7ccccc7)cc6)c6ccccc56)c4c3c2[2H])c([2H])c1[2H] ; [2H]c1c([2H])c(-c2c3ccccc3c(-c3ccccc3-c3ccc(-c4ccccc4)cc3)c3ccccc23)c2c(oc3c([2H])c(-c4ccccc4)c([2H])c([2H])c32)c1[2H]]

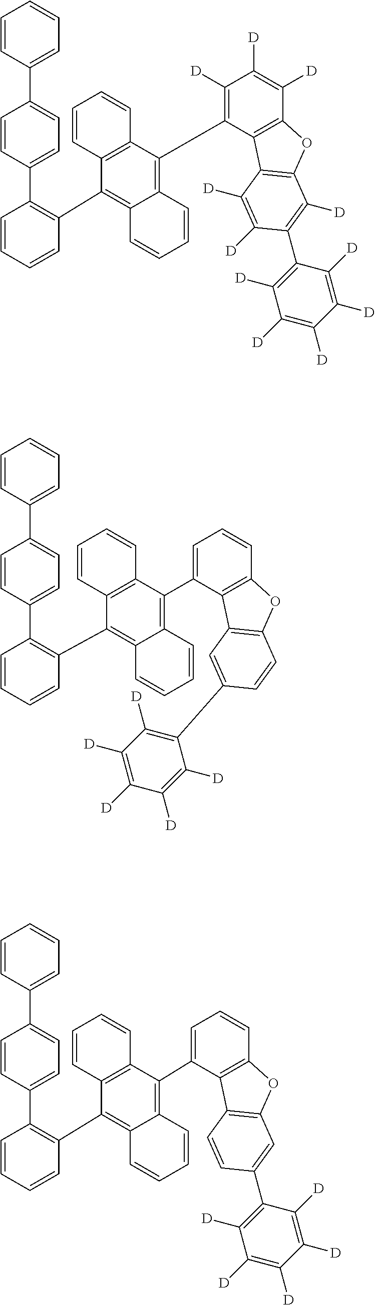 [SMILES: [2H]c1c([2H])c([2H])c(-c2c([2H])c([2H])c3c(oc4c([2H])c([2H])c([2H])c(-c5c6ccccc6c(-c6ccccc6-c6ccc(-c7ccccc7)cc6)c6ccccc56)c43)c2[2H])c([2H])c1[2H] ; [2H]c1c([2H])c([2H])c(-c2ccc3oc4cccc(-c5c6ccccc6c(-c6ccccc6-c6ccc(-c7ccccc7)cc6)c6ccccc56)c4c3c2)c([2H])c1[2H] ; [2H]c1c([2H])c([2H])c(-c2ccc3c(c2)oc2cccc(-c4c5ccccc5c(-c5ccccc5-c5ccc(-c6ccccc6)cc5)c5ccccc45)c23)c([2H])c1[2H]]

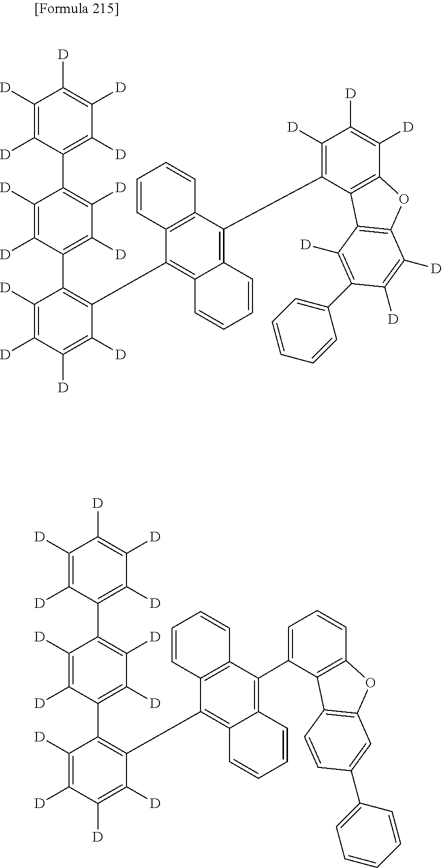 [SMILES: [2H]c1c([2H])c([2H])c(-c2c([2H])c([2H])c(-c3c([2H])c([2H])c([2H])c([2H])c3-c3c4ccccc4c(-c4c([2H])c([2H])c([2H])c5oc6c([2H])c([2H])c(-c7ccccc7)c([2H])c6c45)c4ccccc34)c([2H])c2[2H])c([2H])c1[2H] ; [2H]c1c([2H])c([2H])c(-c2c([2H])c([2H])c(-c3c([2H])c([2H])c([2H])c([2H])c3-c3c4ccccc4c(-c4cccc5oc6cc(-c7ccccc7)ccc6c45)c4ccccc34)c([2H])c2[2H])c([2H])c1[2H]]

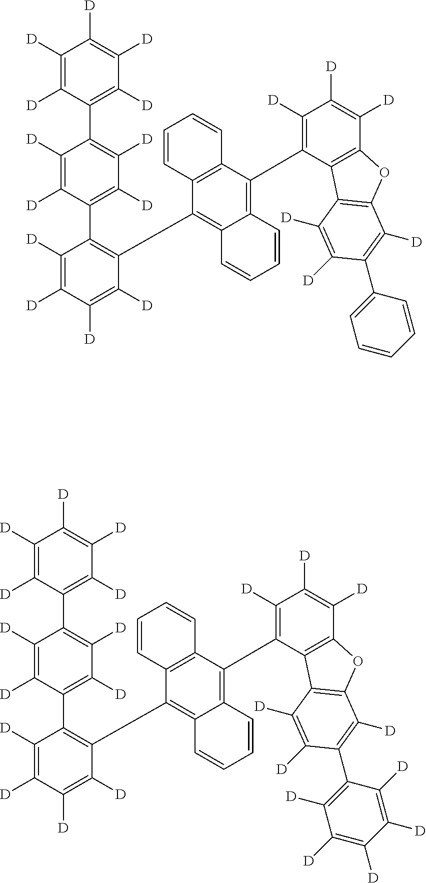 [SMILES: [2H]c1c([2H])c([2H])c(-c2c([2H])c([2H])c(-c3c([2H])c([2H])c([2H])c([2H])c3-c3c4ccccc4c(-c4c([2H])c([2H])c([2H])c5oc6c([2H])c(-c7ccccc7)c([2H])c([2H])c6c45)c4ccccc34)c([2H])c2[2H])c([2H])c1[2H] ; [2H]c1c([2H])c([2H])c(-c2c([2H])c([2H])c(-c3c([2H])c([2H])c([2H])c([2H])c3-c3c4ccccc4c(-c4c([2H])c([2H])c([2H])c5oc6c([2H])c(-c7c([2H])c([2H])c([2H])c([2H])c7[2H])c([2H])c([2H])c6c45)c4ccccc34)c([2H])c2[2H])c([2H])c1[2H]]

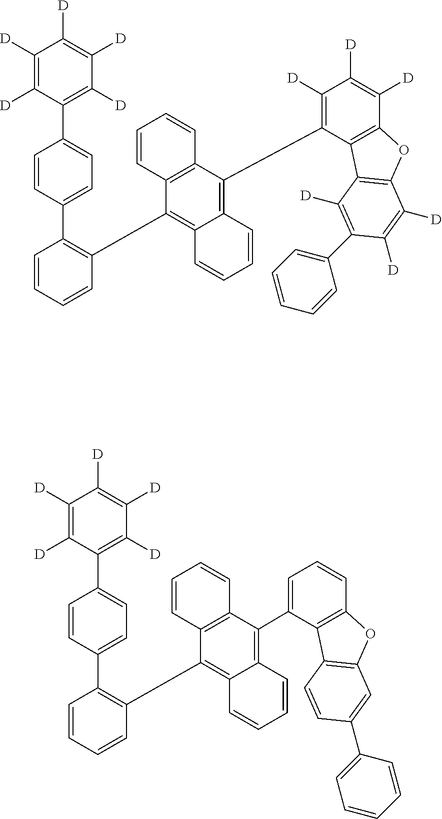 [SMILES: [2H]c1c([2H])c([2H])c(-c2ccc(-c3ccccc3-c3c4ccccc4c(-c4c([2H])c([2H])c([2H])c5oc6c([2H])c([2H])c(-c7ccccc7)c([2H])c6c45)c4ccccc34)cc2)c([2H])c1[2H] ; [2H]c1c([2H])c([2H])c(-c2ccc(-c3ccccc3-c3c4ccccc4c(-c4cccc5oc6cc(-c7ccccc7)ccc6c45)c4ccccc34)cc2)c([2H])c1[2H]]

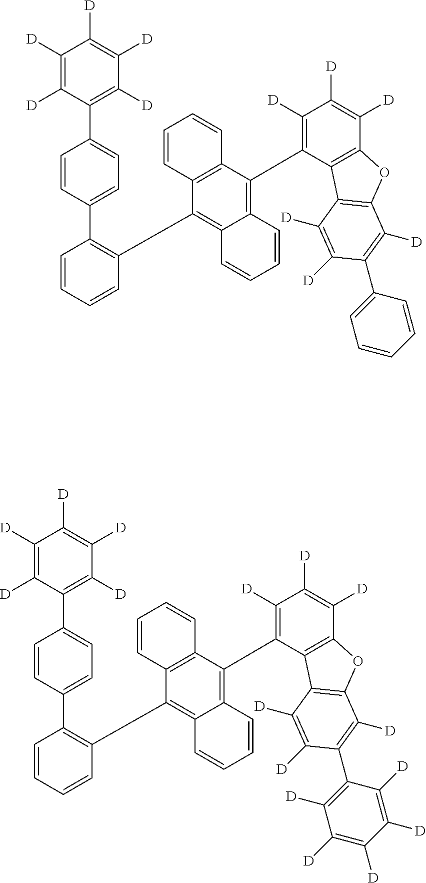 [SMILES: [2H]c1c([2H])c([2H])c(-c2ccc(-c3ccccc3-c3c4ccccc4c(-c4c([2H])c([2H])c([2H])c5oc6c([2H])c(-c7ccccc7)c([2H])c([2H])c6c45)c4ccccc34)cc2)c([2H])c1[2H] ; [2H]c1c([2H])c([2H])c(-c2ccc(-c3ccccc3-c3c4ccccc4c(-c4c([2H])c([2H])c([2H])c5oc6c([2H])c(-c7c([2H])c([2H])c([2H])c([2H])c7[2H])c([2H])c([2H])c6c45)c4ccccc34)cc2)c([2H])c1[2H]]

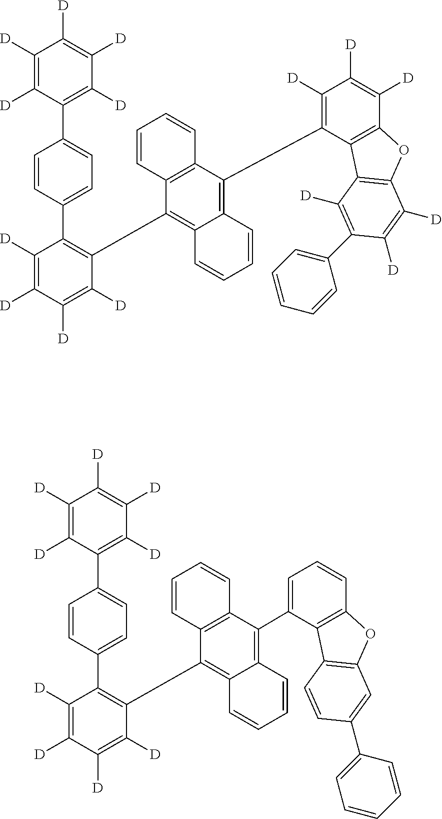 [SMILES: [2H]c1c([2H])c([2H])c(-c2ccc(-c3c([2H])c([2H])c([2H])c([2H])c3-c3c4ccccc4c(-c4c([2H])c([2H])c([2H])c5oc6c([2H])c([2H])c(-c7ccccc7)c([2H])c6c45)c4ccccc34)cc2)c([2H])c1[2H] ; [2H]c1c([2H])c([2H])c(-c2ccc(-c3c([2H])c([2H])c([2H])c([2H])c3-c3c4ccccc4c(-c4cccc5oc6cc(-c7ccccc7)ccc6c45)c4ccccc34)cc2)c([2H])c1[2H]]

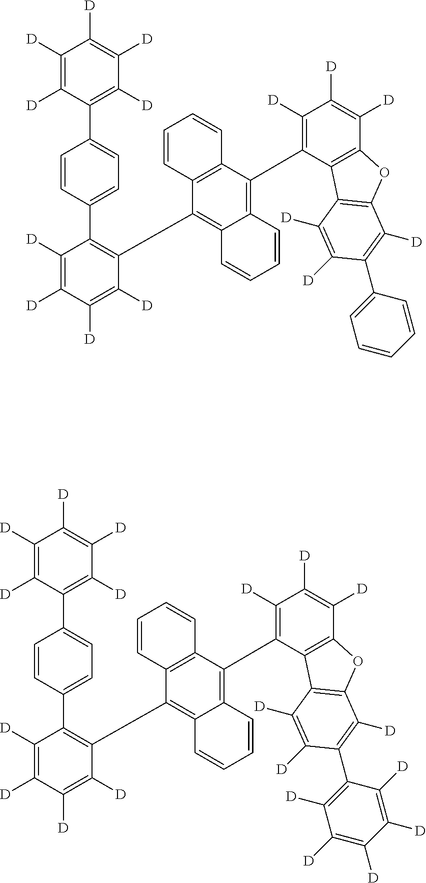 [SMILES: [2H]c1c([2H])c([2H])c(-c2ccc(-c3c([2H])c([2H])c([2H])c([2H])c3-c3c4ccccc4c(-c4c([2H])c([2H])c([2H])c5oc6c([2H])c(-c7ccccc7)c([2H])c([2H])c6c45)c4ccccc34)cc2)c([2H])c1[2H] ; [2H]c1c([2H])c([2H])c(-c2ccc(-c3c([2H])c([2H])c([2H])c([2H])c3-c3c4ccccc4c(-c4c([2H])c([2H])c([2H])c5oc6c([2H])c(-c7c([2H])c([2H])c([2H])c([2H])c7[2H])c([2H])c([2H])c6c45)c4ccccc34)cc2)c([2H])c1[2H]]

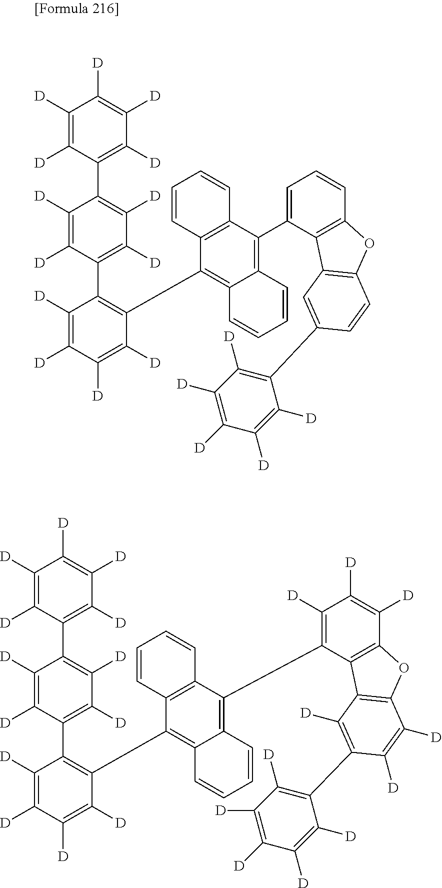 [SMILES: [2H]c1c([2H])c([2H])c(-c2ccc3oc4cccc(-c5c6ccccc6c(-c6c([2H])c([2H])c([2H])c([2H])c6-c6c([2H])c([2H])c(-c7c([2H])c([2H])c([2H])c([2H])c7[2H])c([2H])c6[2H])c6ccccc56)c4c3c2)c([2H])c1[2H] ; [2H]c1c([2H])c([2H])c(-c2c([2H])c([2H])c(-c3c([2H])c([2H])c([2H])c([2H])c3-c3c4ccccc4c(-c4c([2H])c([2H])c([2H])c5oc6c([2H])c([2H])c(-c7c([2H])c([2H])c([2H])c([2H])c7[2H])c([2H])c6c45)c4ccccc34)c([2H])c2[2H])c([2H])c1[2H]]

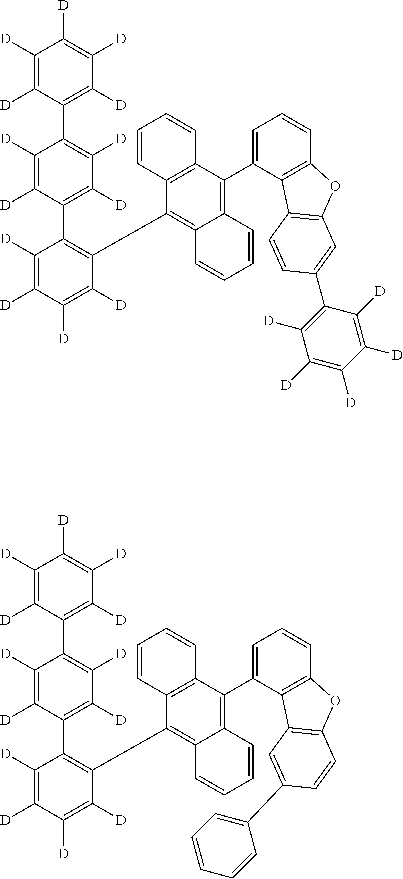 [SMILES: [2H]c1c([2H])c([2H])c(-c2ccc3c(c2)oc2cccc(-c4c5ccccc5c(-c5c([2H])c([2H])c([2H])c([2H])c5-c5c([2H])c([2H])c(-c6c([2H])c([2H])c([2H])c([2H])c6[2H])c([2H])c5[2H])c5ccccc45)c23)c([2H])c1[2H] ; [2H]c1c([2H])c([2H])c(-c2c([2H])c([2H])c(-c3c([2H])c([2H])c([2H])c([2H])c3-c3c4ccccc4c(-c4cccc5oc6ccc(-c7ccccc7)cc6c45)c4ccccc34)c([2H])c2[2H])c([2H])c1[2H]]

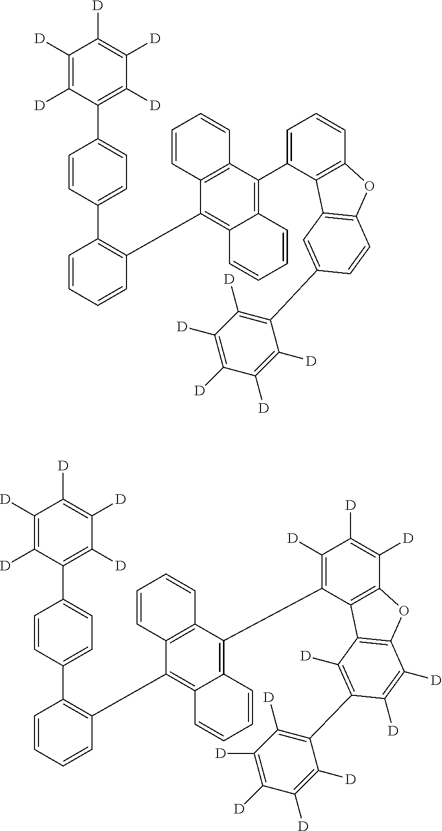 [SMILES: [2H]c1c([2H])c([2H])c(-c2ccc(-c3ccccc3-c3c4ccccc4c(-c4cccc5oc6ccc(-c7c([2H])c([2H])c([2H])c([2H])c7[2H])cc6c45)c4ccccc34)cc2)c([2H])c1[2H] ; [2H]c1c([2H])c([2H])c(-c2ccc(-c3ccccc3-c3c4ccccc4c(-c4c([2H])c([2H])c([2H])c5oc6c([2H])c([2H])c(-c7c([2H])c([2H])c([2H])c([2H])c7[2H])c([2H])c6c45)c4ccccc34)cc2)c([2H])c1[2H]]

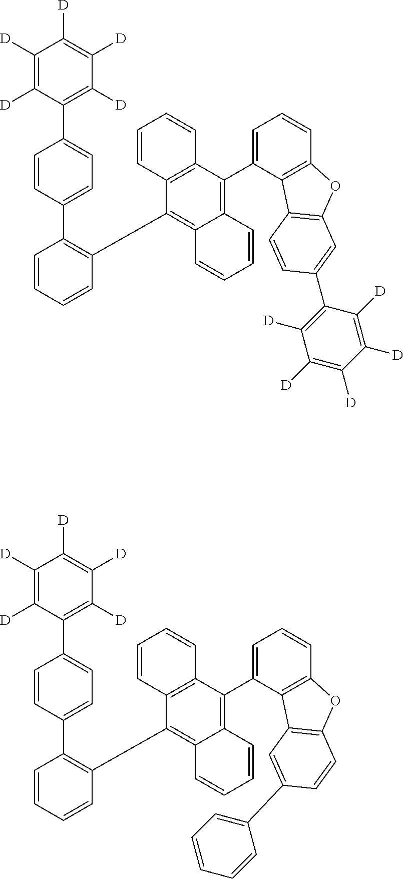 [SMILES: [2H]c1c([2H])c([2H])c(-c2ccc(-c3ccccc3-c3c4ccccc4c(-c4cccc5oc6cc(-c7c([2H])c([2H])c([2H])c([2H])c7[2H])ccc6c45)c4ccccc34)cc2)c([2H])c1[2H] ; [2H]c1c([2H])c([2H])c(-c2ccc(-c3ccccc3-c3c4ccccc4c(-c4cccc5oc6ccc(-c7ccccc7)cc6c45)c4ccccc34)cc2)c([2H])c1[2H]]

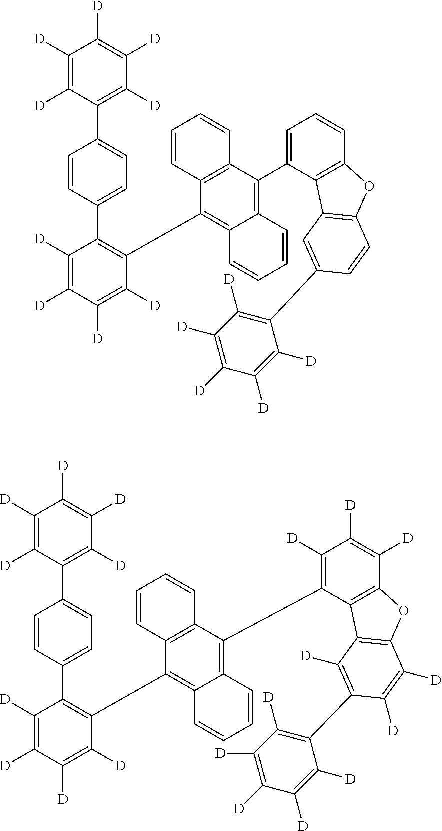 [SMILES: [2H]c1c([2H])c([2H])c(-c2ccc(-c3c([2H])c([2H])c([2H])c([2H])c3-c3c4ccccc4c(-c4cccc5oc6ccc(-c7c([2H])c([2H])c([2H])c([2H])c7[2H])cc6c45)c4ccccc34)cc2)c([2H])c1[2H] ; [2H]c1c([2H])c([2H])c(-c2ccc(-c3c([2H])c([2H])c([2H])c([2H])c3-c3c4ccccc4c(-c4c([2H])c([2H])c([2H])c5oc6c([2H])c([2H])c(-c7c([2H])c([2H])c([2H])c([2H])c7[2H])c([2H])c6c45)c4ccccc34)cc2)c([2H])c1[2H]]

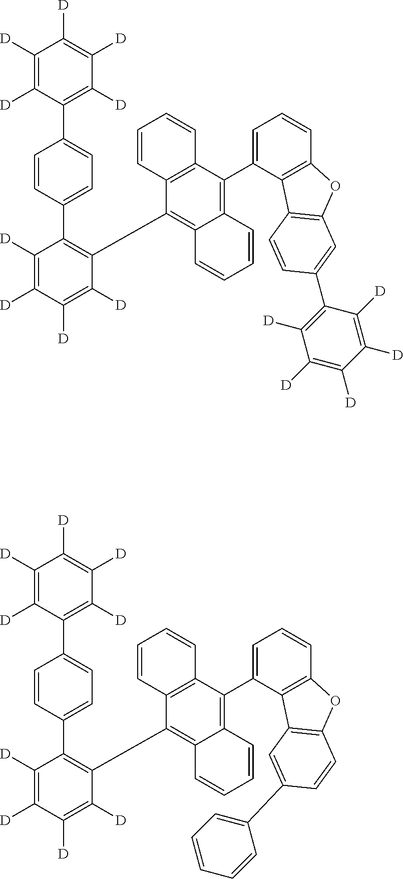 [SMILES: [2H]c1c([2H])c([2H])c(-c2ccc(-c3c([2H])c([2H])c([2H])c([2H])c3-c3c4ccccc4c(-c4cccc5oc6cc(-c7c([2H])c([2H])c([2H])c([2H])c7[2H])ccc6c45)c4ccccc34)cc2)c([2H])c1[2H] ; [2H]c1c([2H])c([2H])c(-c2ccc(-c3c([2H])c([2H])c([2H])c([2H])c3-c3c4ccccc4c(-c4cccc5oc6ccc(-c7ccccc7)cc6c45)c4ccccc34)cc2)c([2H])c1[2H]]

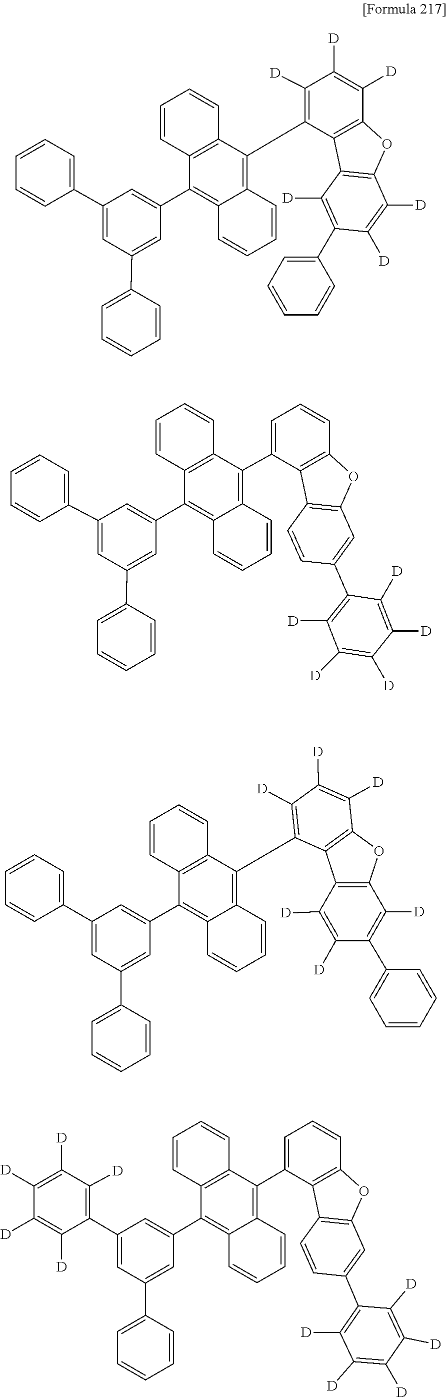

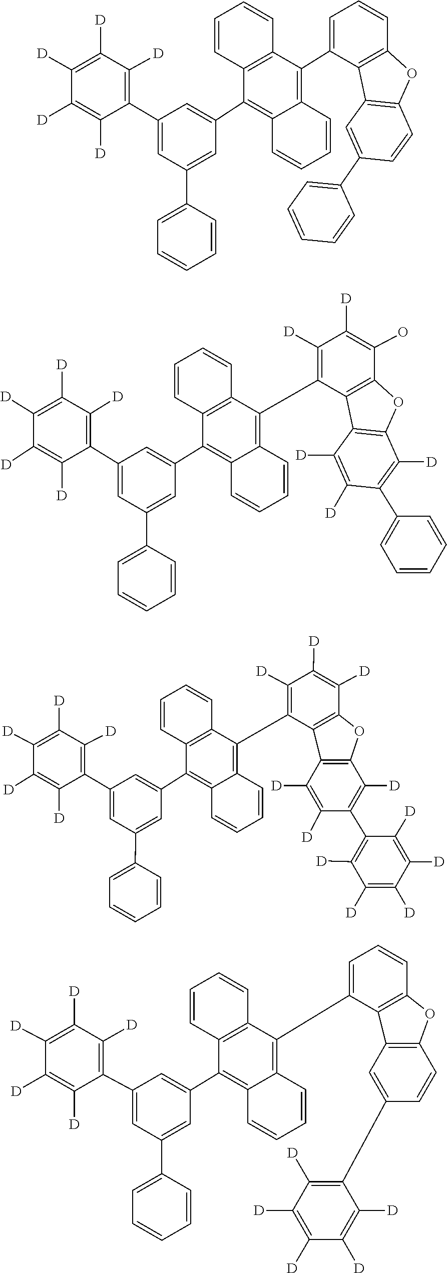

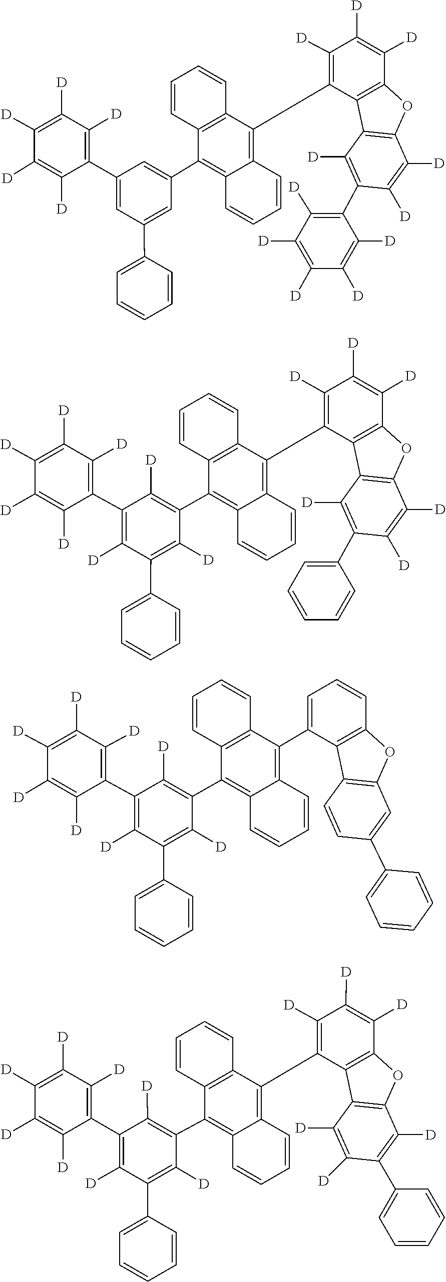

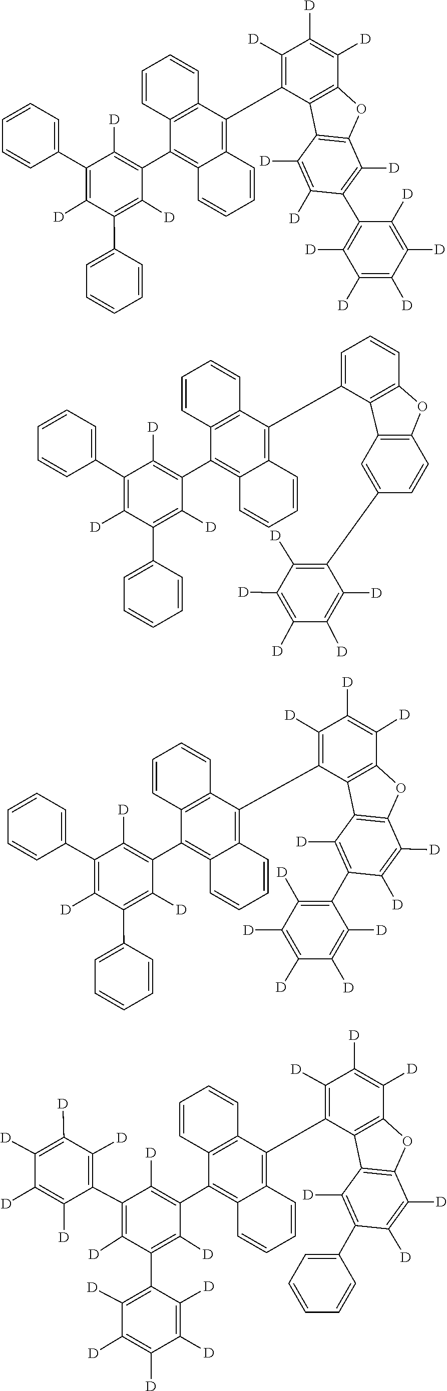

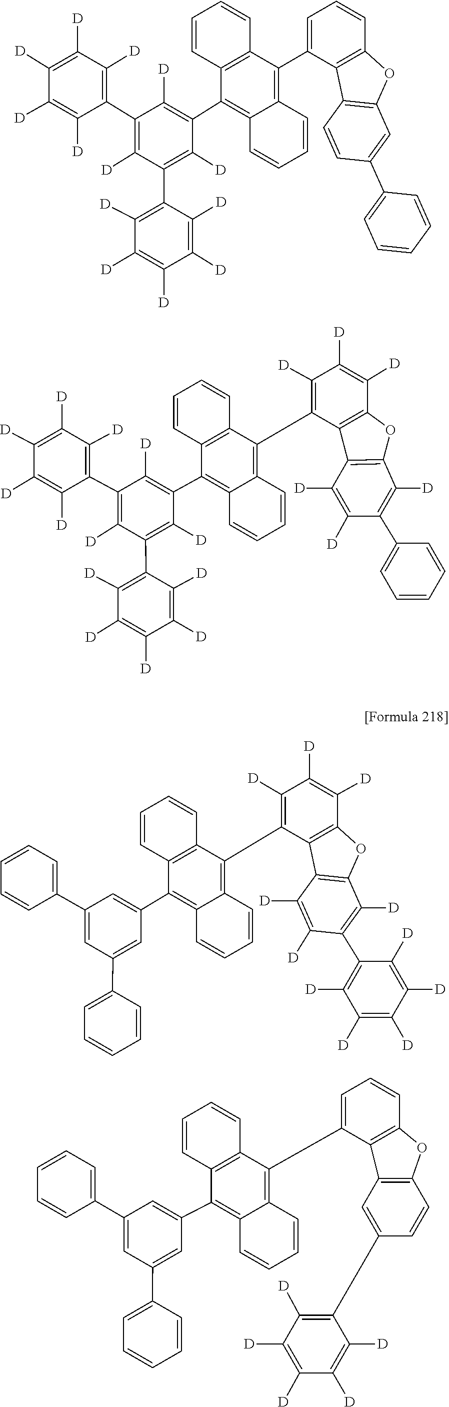

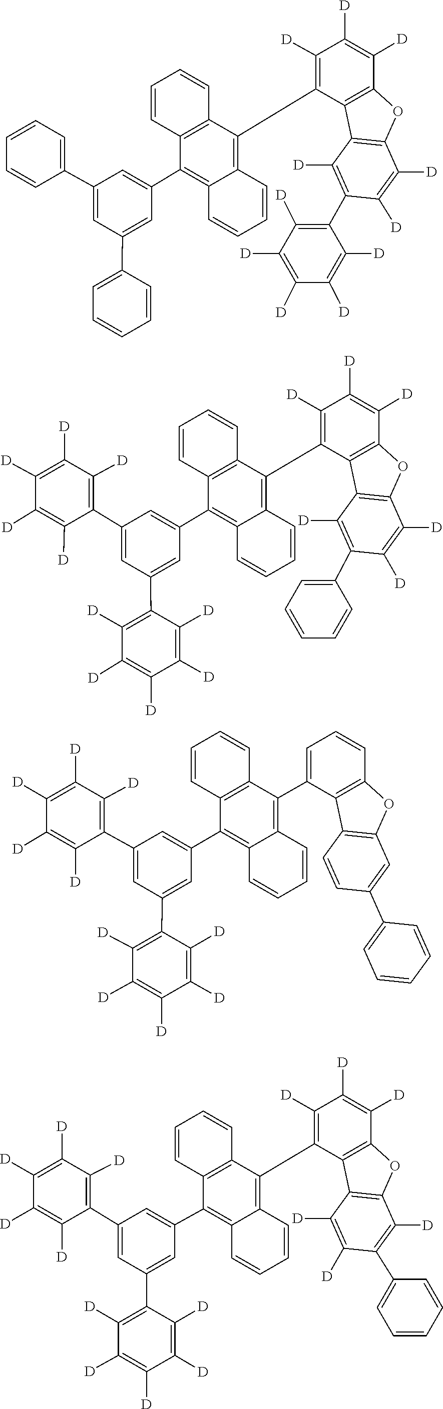

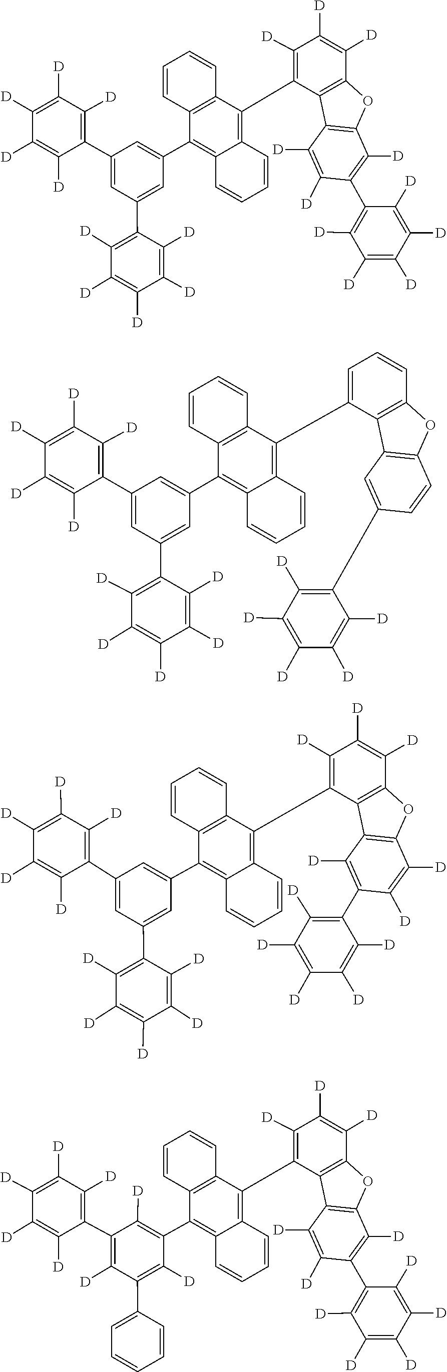

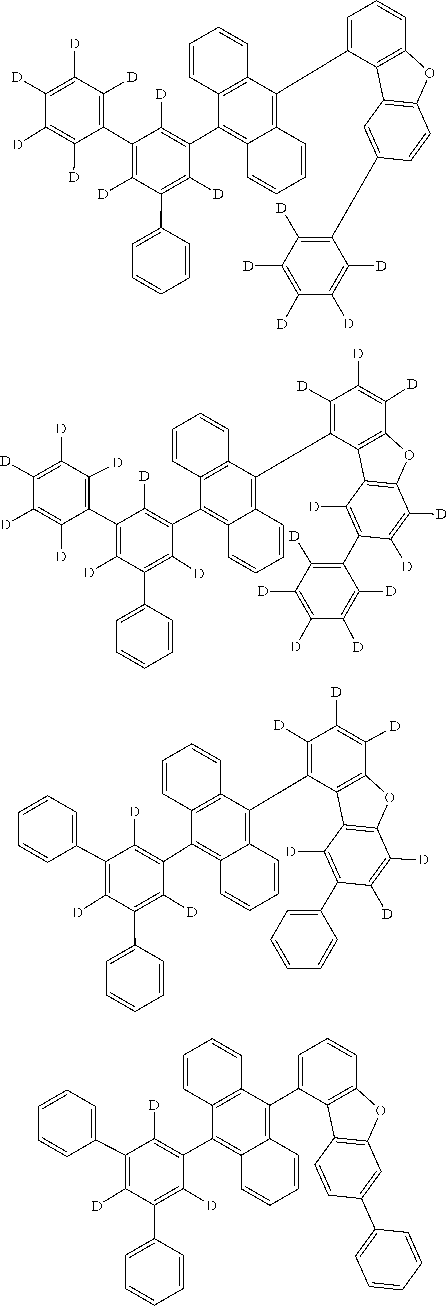

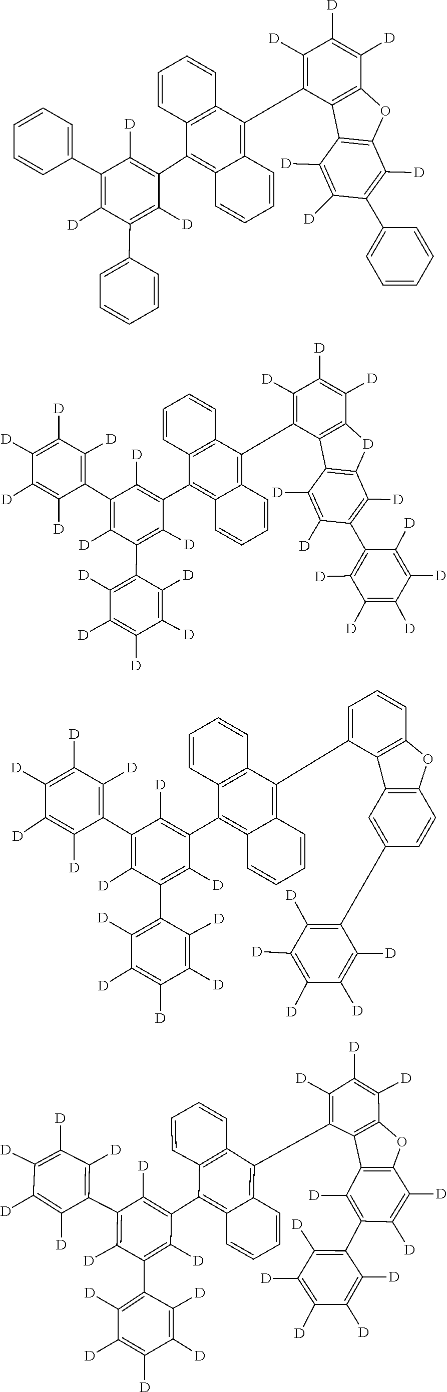

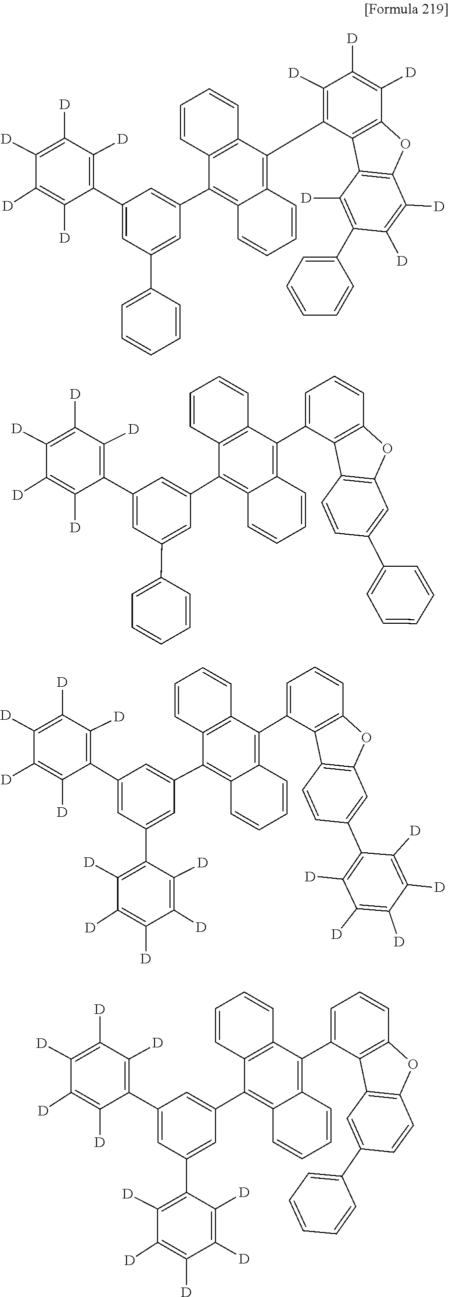

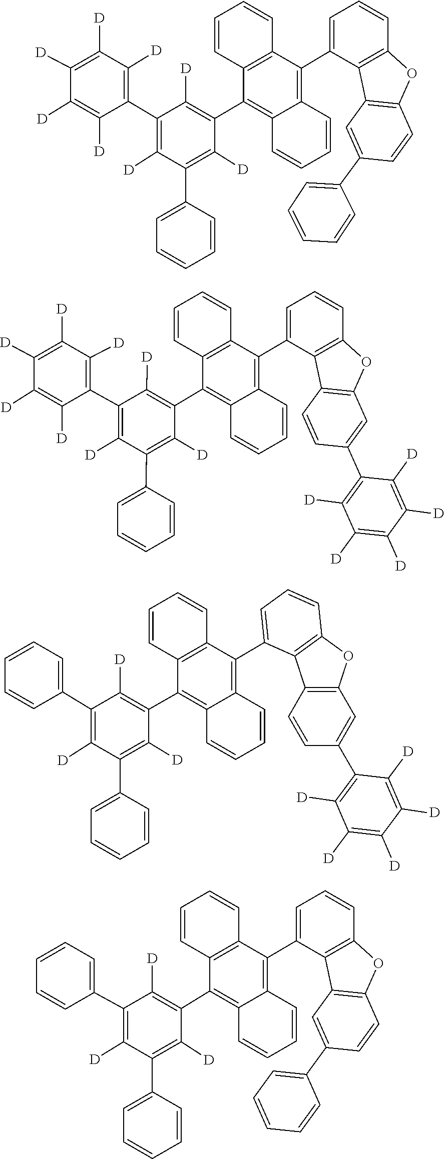

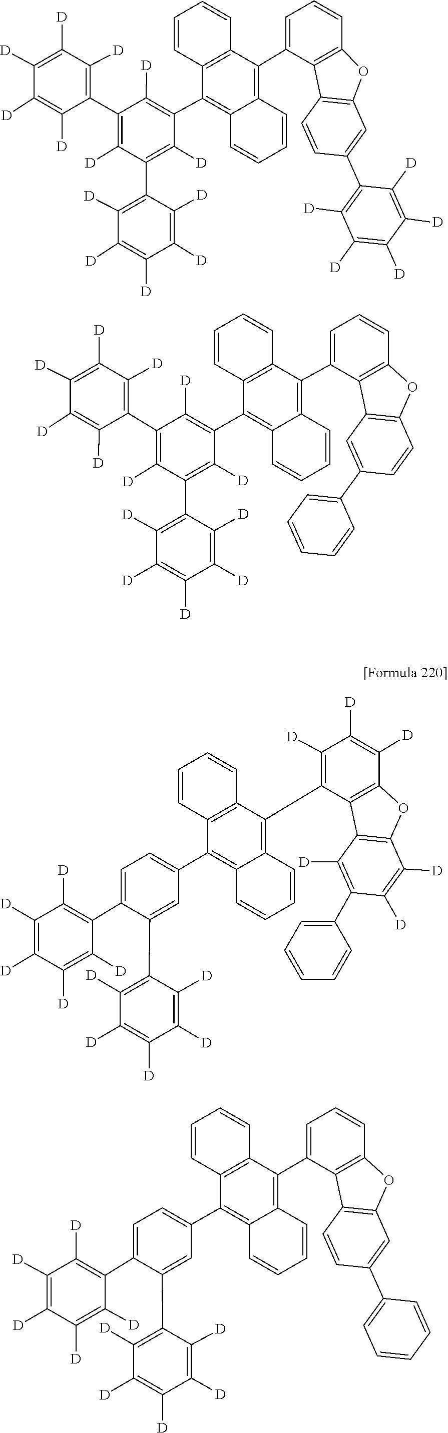

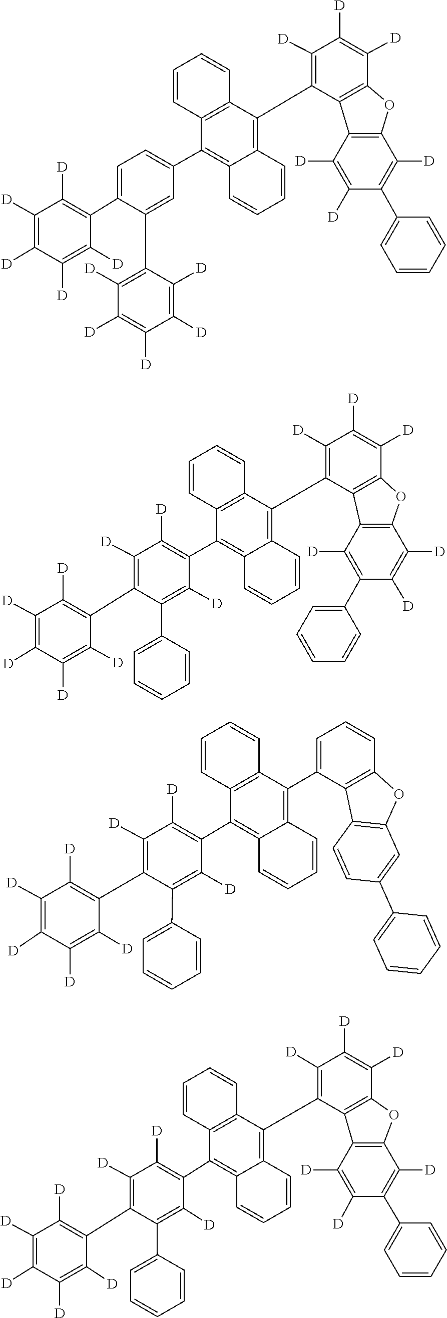

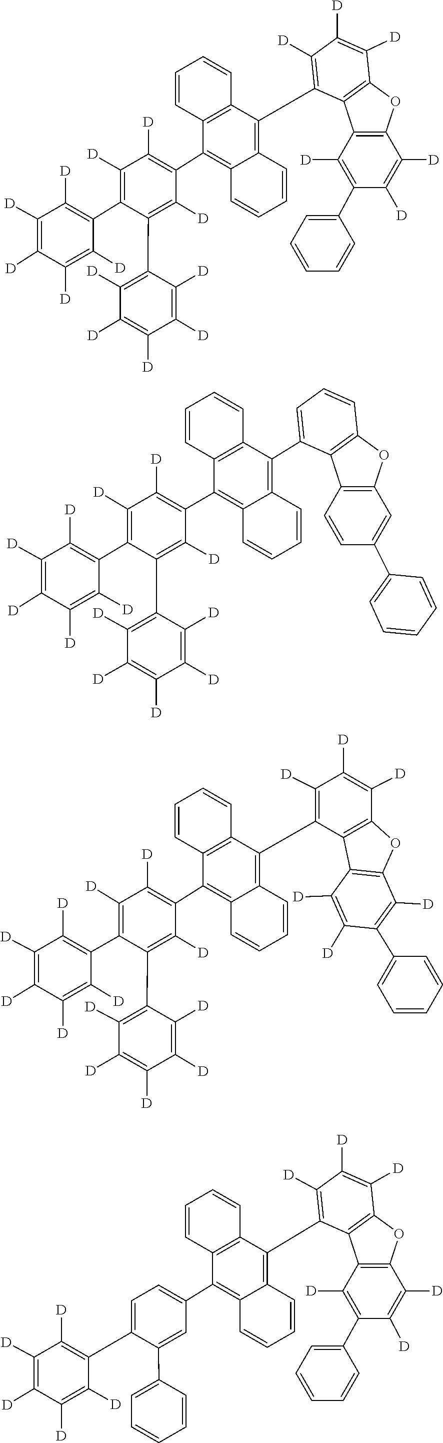

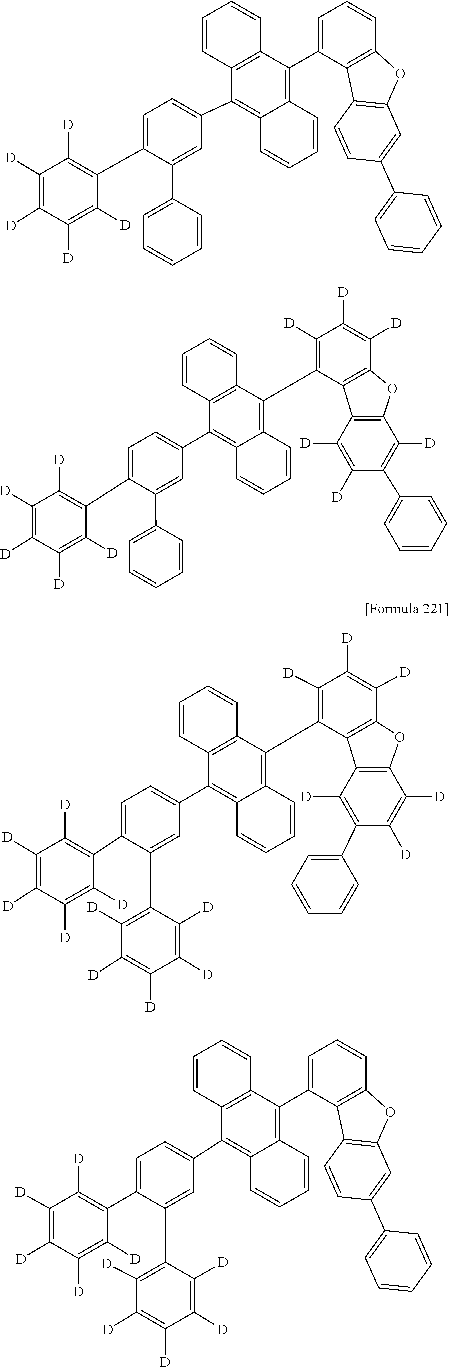

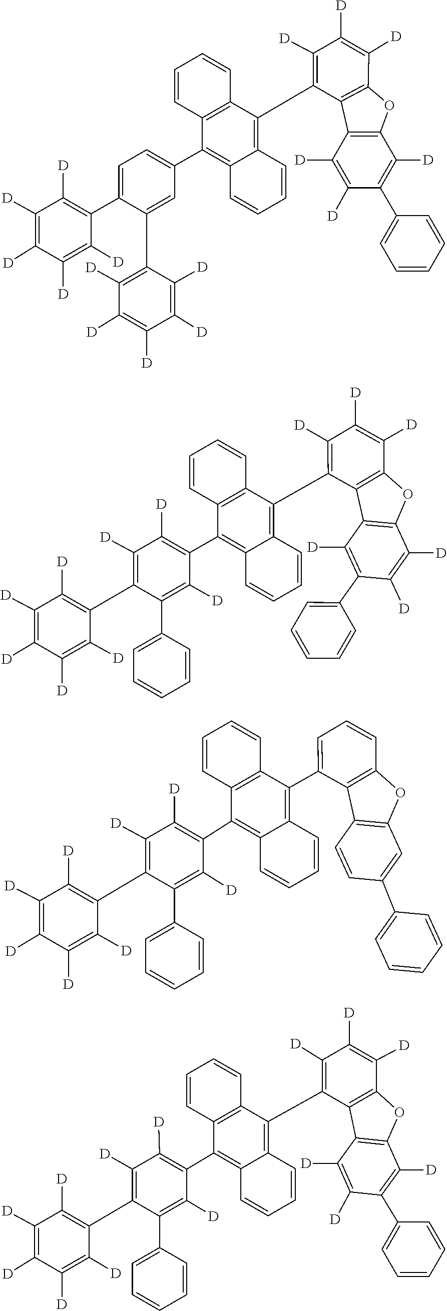

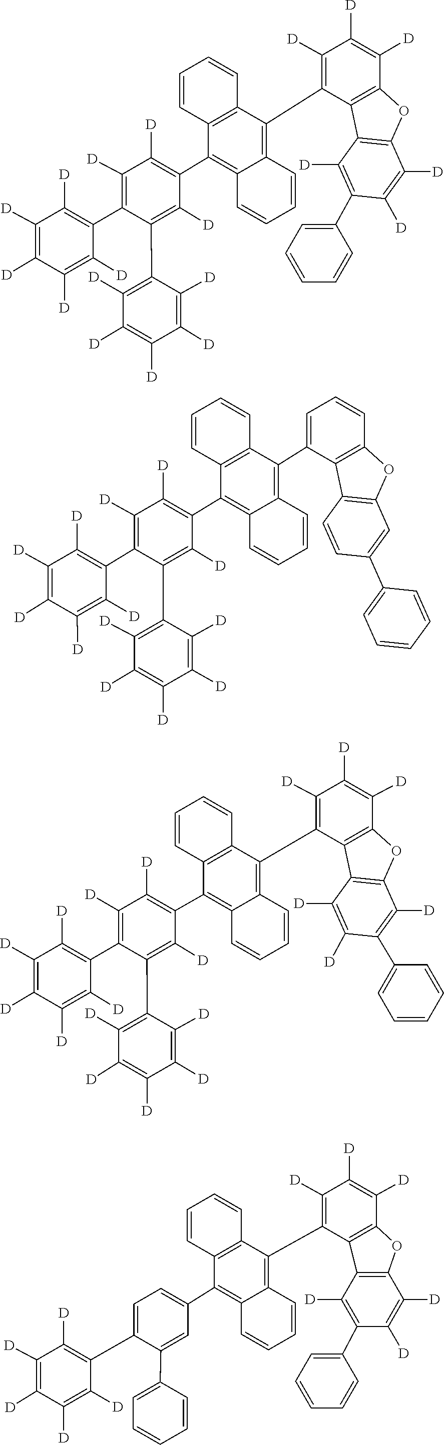

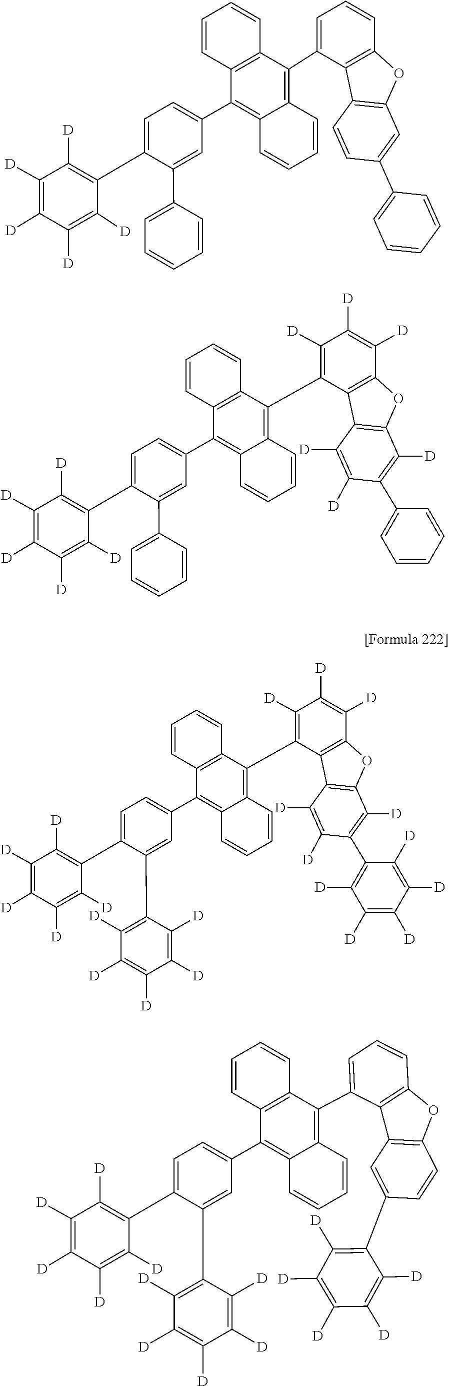

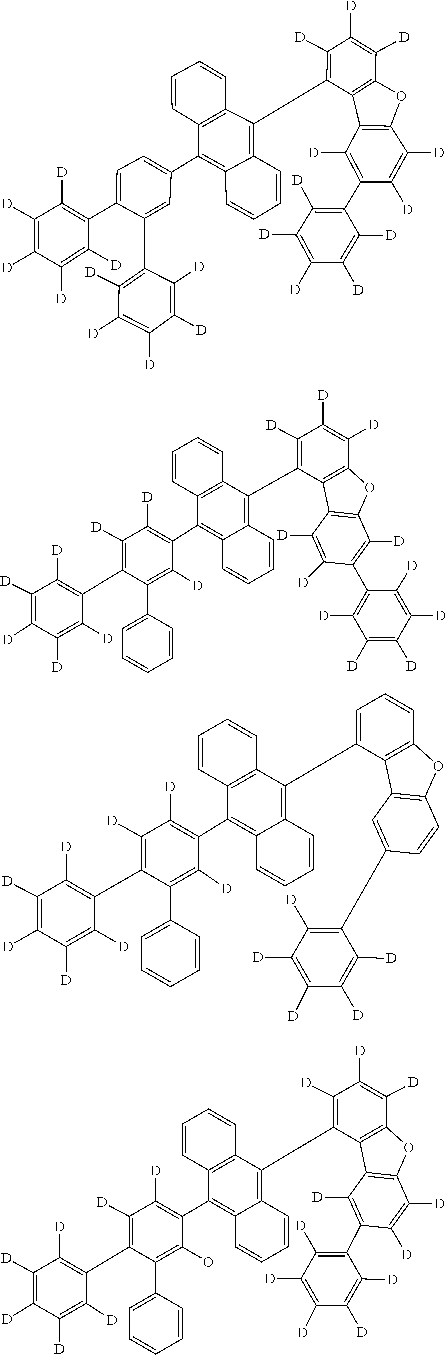

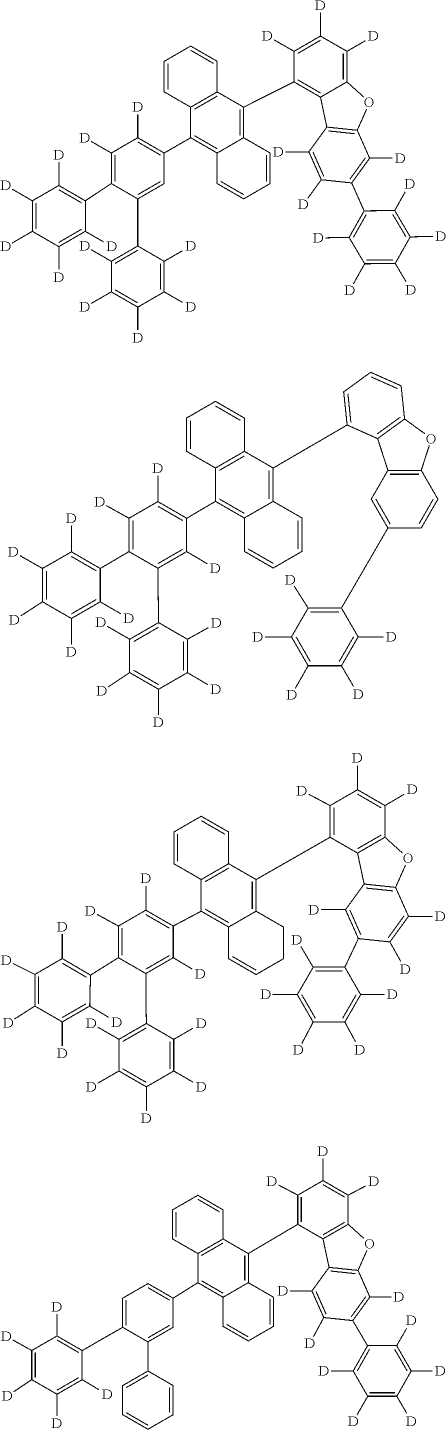

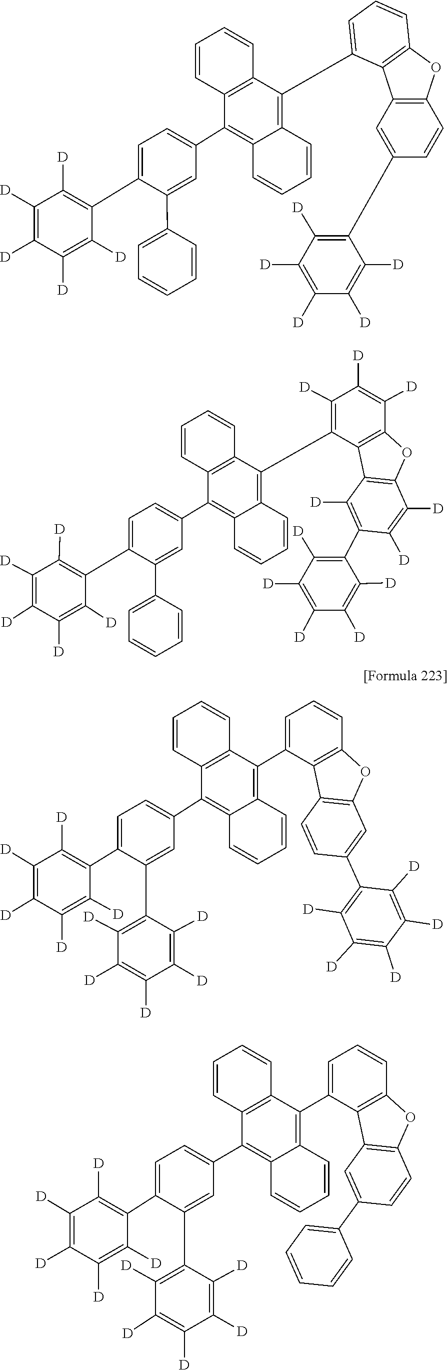

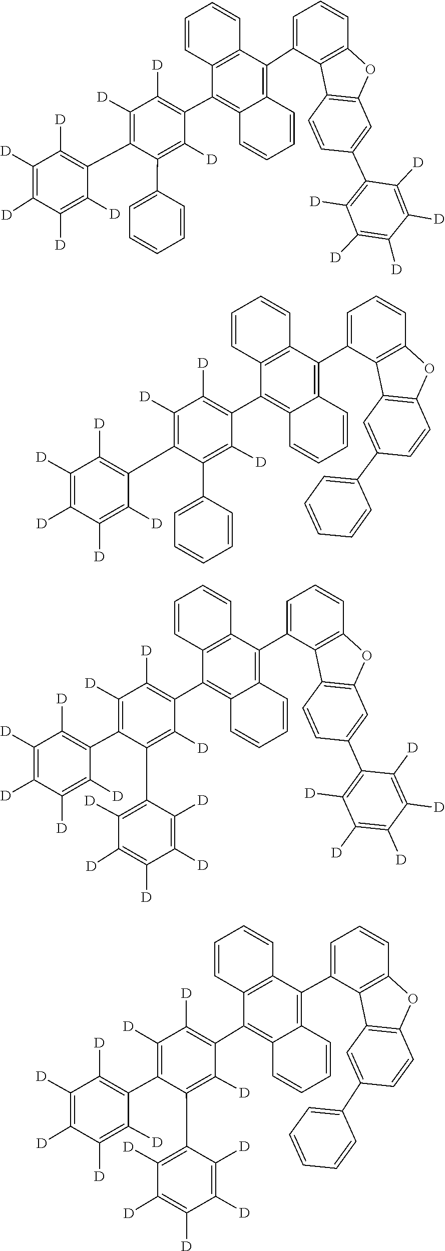

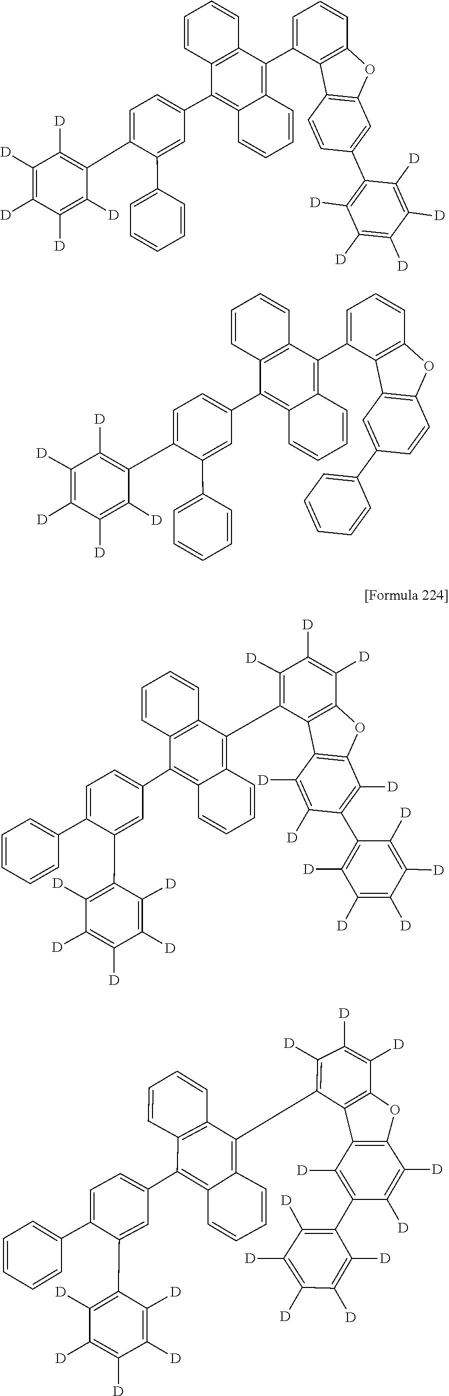

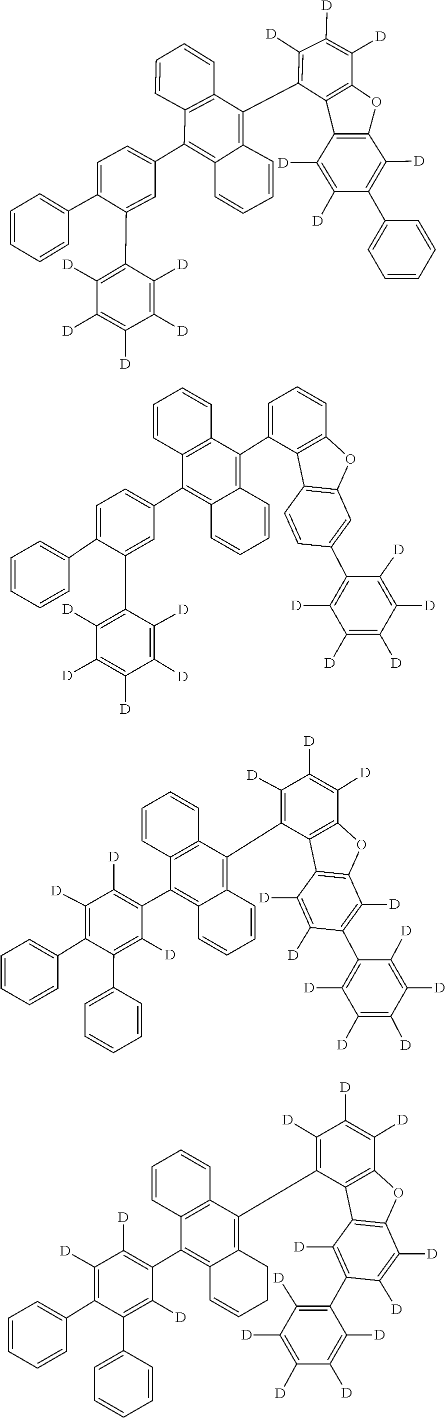

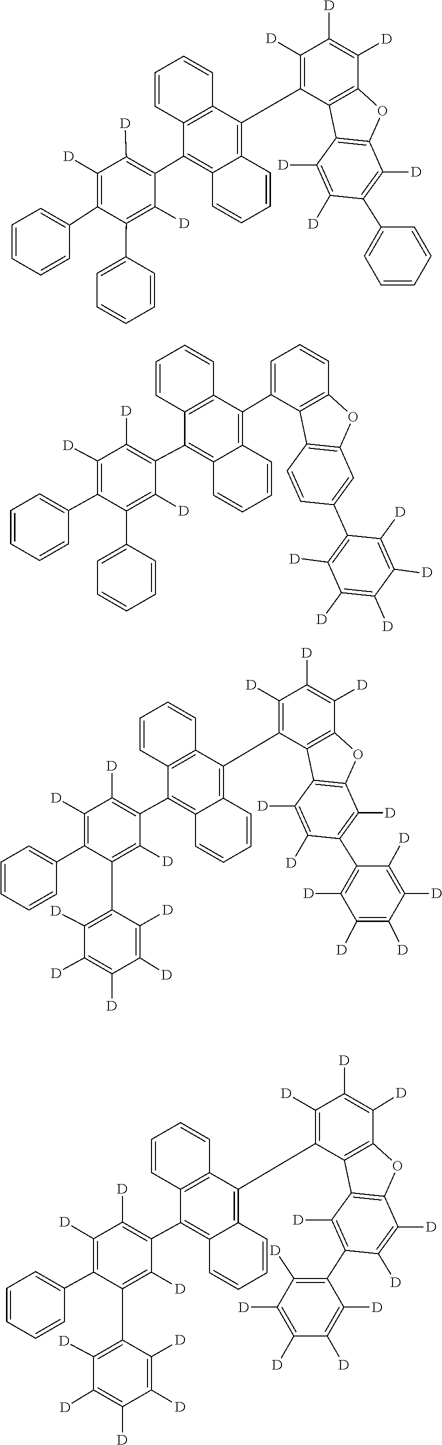

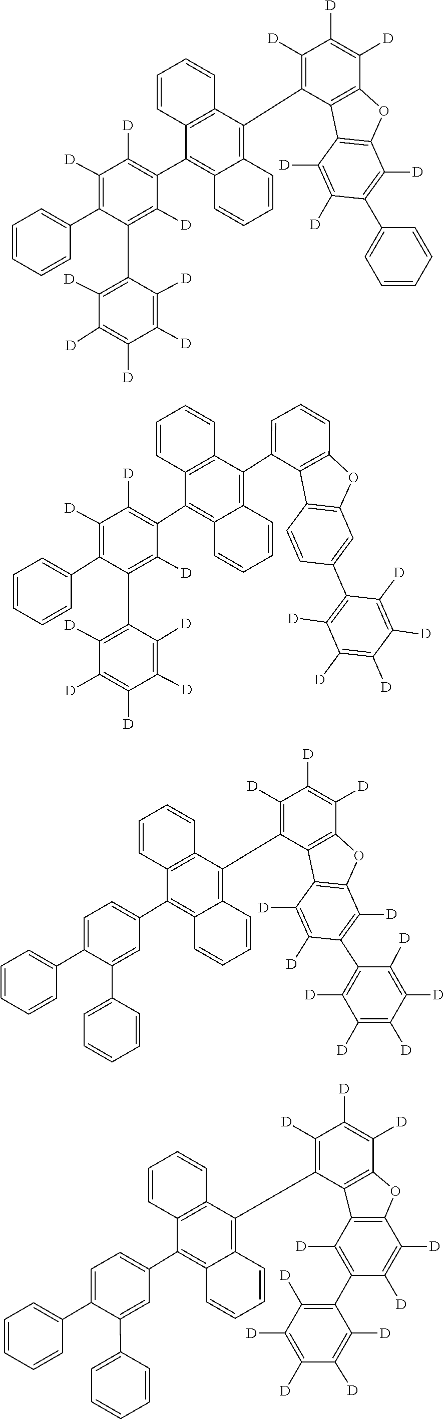

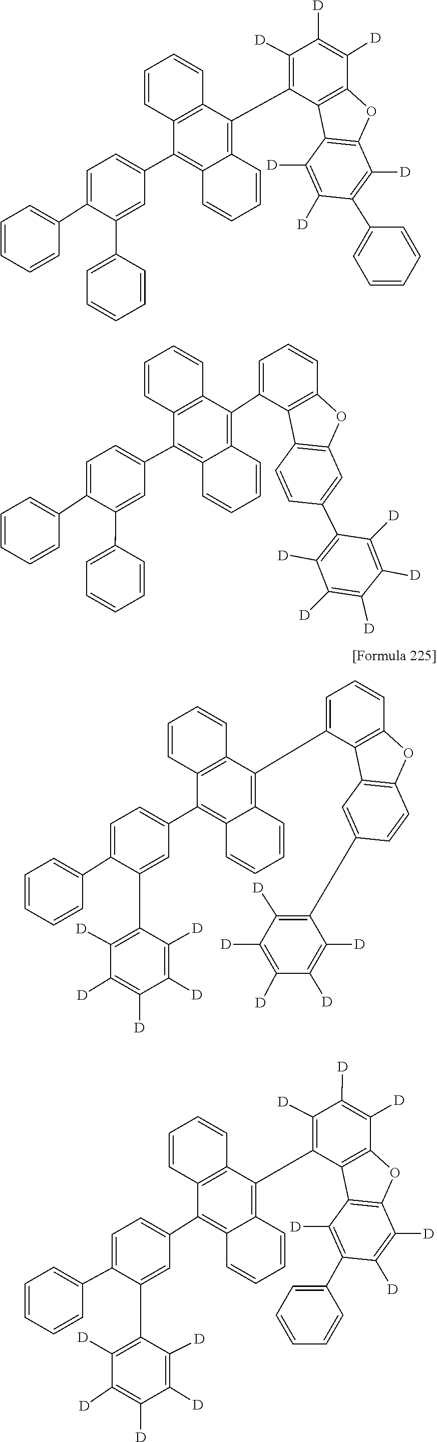

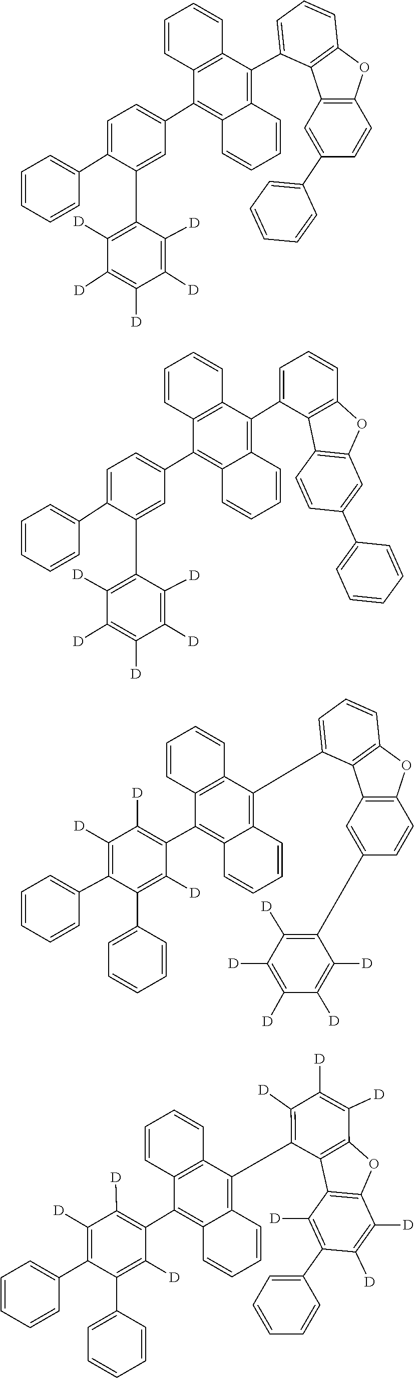

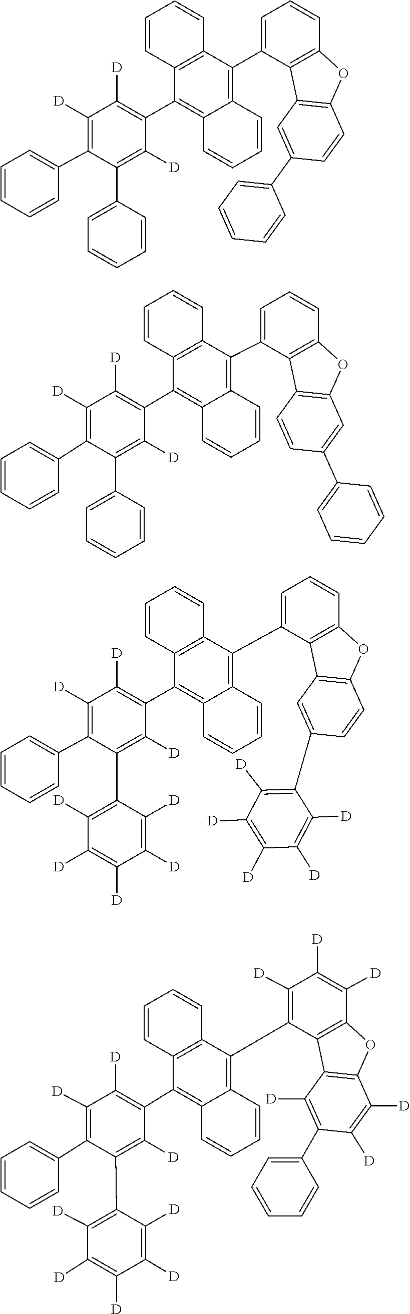

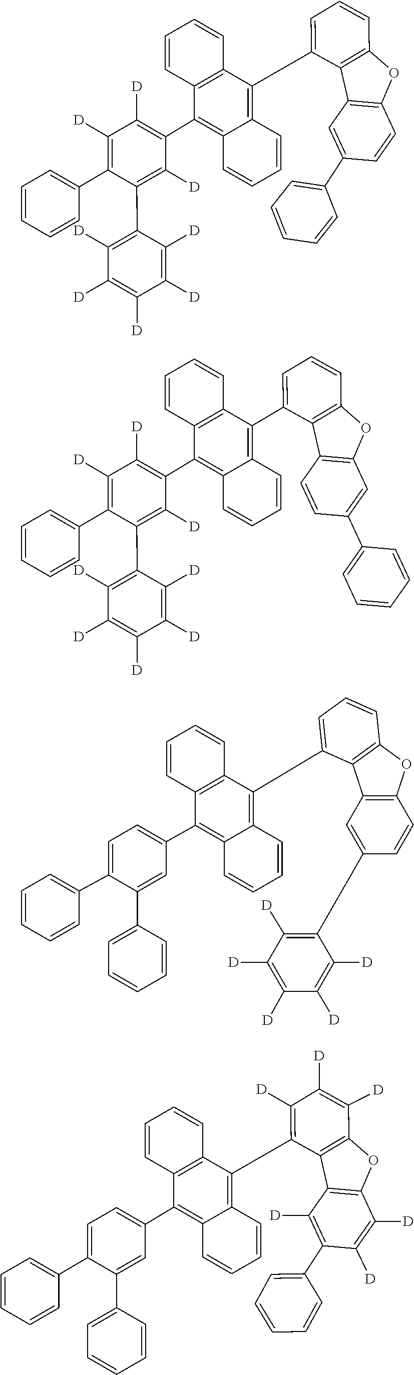

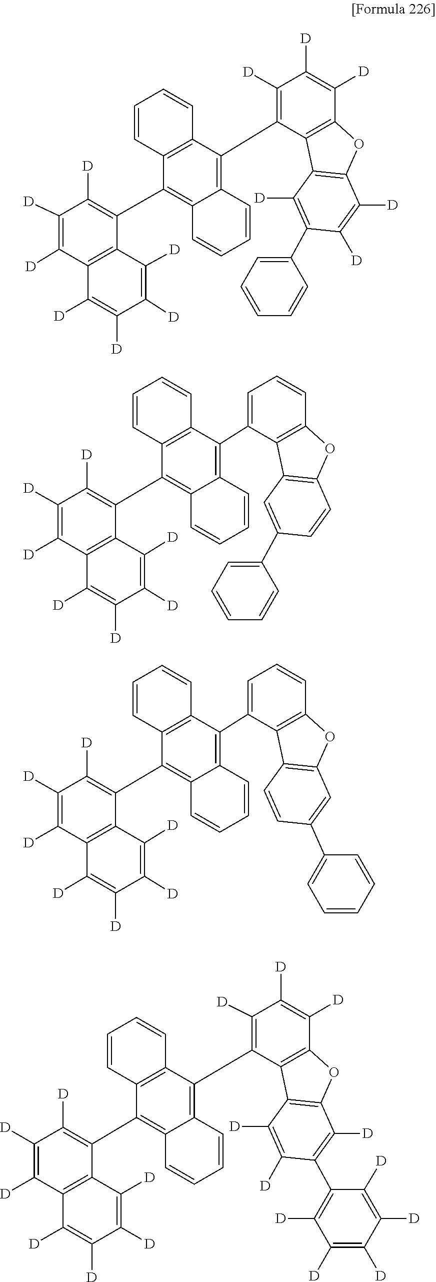

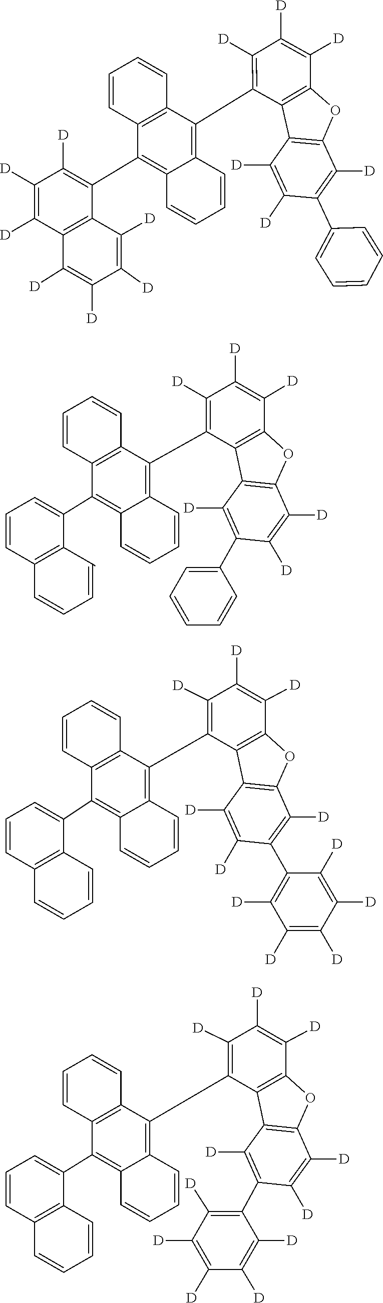

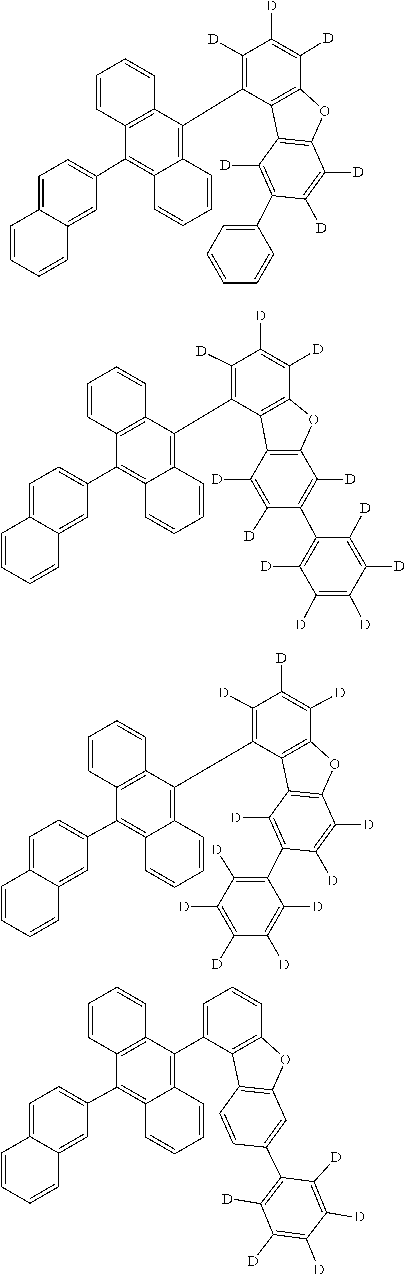

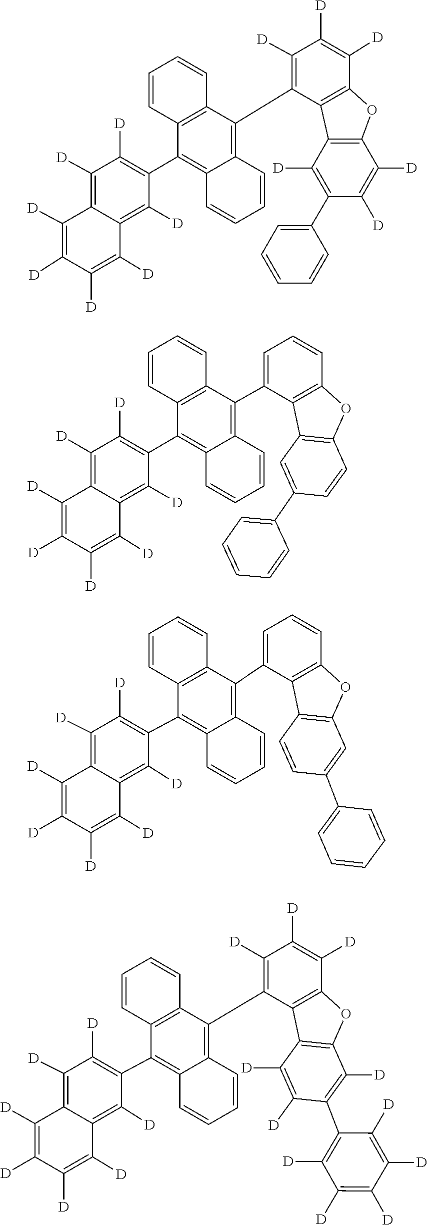

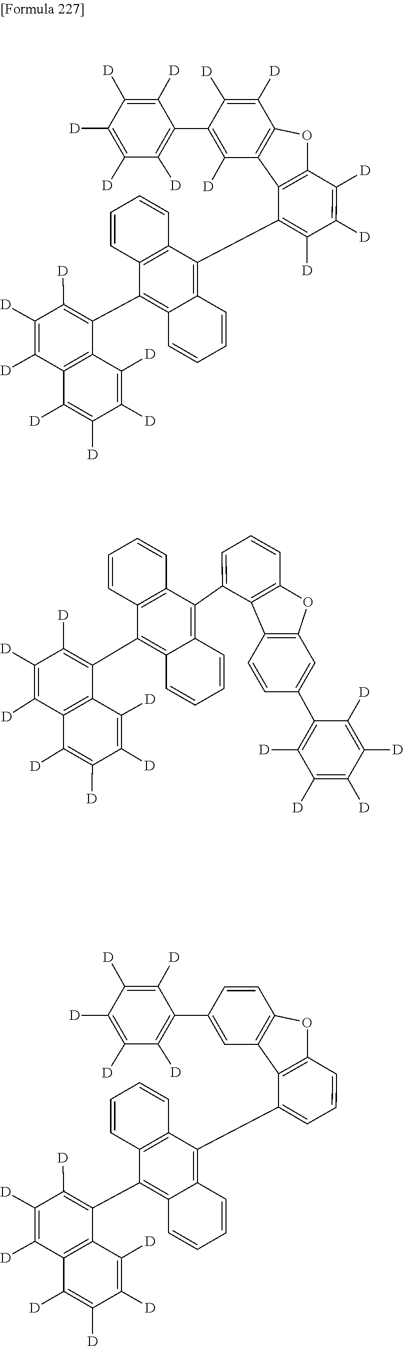 [SMILES: [2H]c1c([2H])c([2H])c(-c2c([2H])c([2H])c3oc4c([2H])c([2H])c([2H])c(-c5c6ccccc6c(-c6c([2H])c([2H])c([2H])c7c([2H])c([2H])c([2H])c([2H])c67)c6ccccc56)c4c3c2[2H])c([2H])c1[2H] ; [2H]c1c([2H])c([2H])c(-c2ccc3c(c2)oc2cccc(-c4c5ccccc5c(-c5c([2H])c([2H])c([2H])c6c([2H])c([2H])c([2H])c([2H])c56)c5ccccc45)c23)c([2H])c1[2H] ; [2H]c1c([2H])c([2H])c(-c2ccc3oc4cccc(-c5c6ccccc6c(-c6c([2H])c([2H])c([2H])c7c([2H])c([2H])c([2H])c([2H])c67)c6ccccc56)c4c3c2)c([2H])c1[2H]]

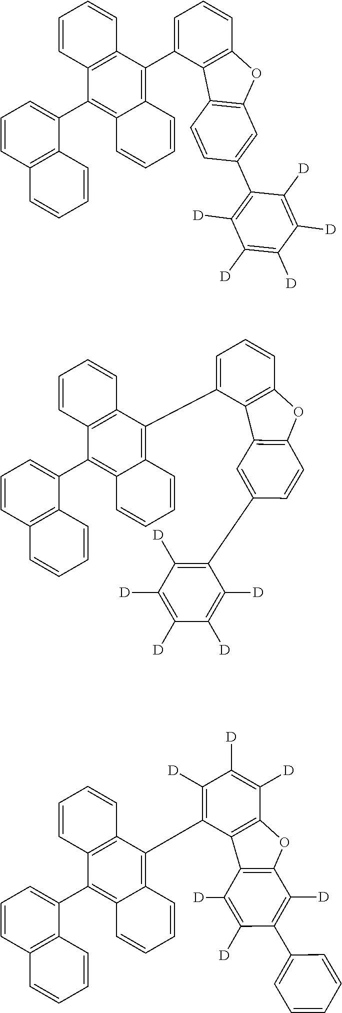 [SMILES: [2H]c1c([2H])c([2H])c(-c2ccc3c(c2)oc2cccc(-c4c5ccccc5c(-c5cccc6ccccc56)c5ccccc45)c23)c([2H])c1[2H] ; [2H]c1c([2H])c([2H])c(-c2ccc3oc4cccc(-c5c6ccccc6c(-c6cccc7ccccc67)c6ccccc56)c4c3c2)c([2H])c1[2H] ; [2H]c1c([2H])c(-c2c3ccccc3c(-c3cccc4ccccc34)c3ccccc23)c2c(oc3c([2H])c(-c4ccccc4)c([2H])c([2H])c32)c1[2H]]

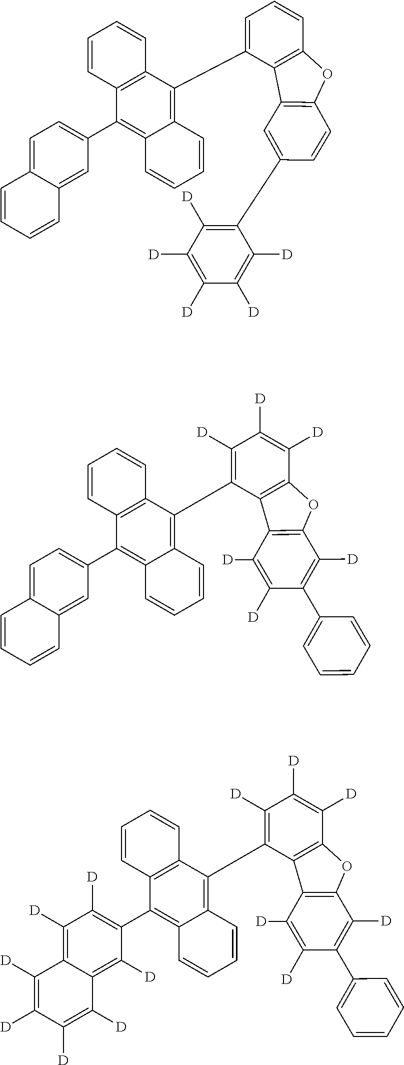 [SMILES: [2H]c1c([2H])c([2H])c(-c2ccc3oc4cccc(-c5c6ccccc6c(-c6ccc7ccccc7c6)c6ccccc56)c4c3c2)c([2H])c1[2H] ; [2H]c1c([2H])c(-c2c3ccccc3c(-c3ccc4ccccc4c3)c3ccccc23)c2c(oc3c([2H])c(-c4ccccc4)c([2H])c([2H])c32)c1[2H] ; [2H]c1c([2H])c(-c2c3ccccc3c(-c3c([2H])c([2H])c4c([2H])c([2H])c([2H])c([2H])c4c3[2H])c3ccccc23)c2c(oc3c([2H])c(-c4ccccc4)c([2H])c([2H])c32)c1[2H]]

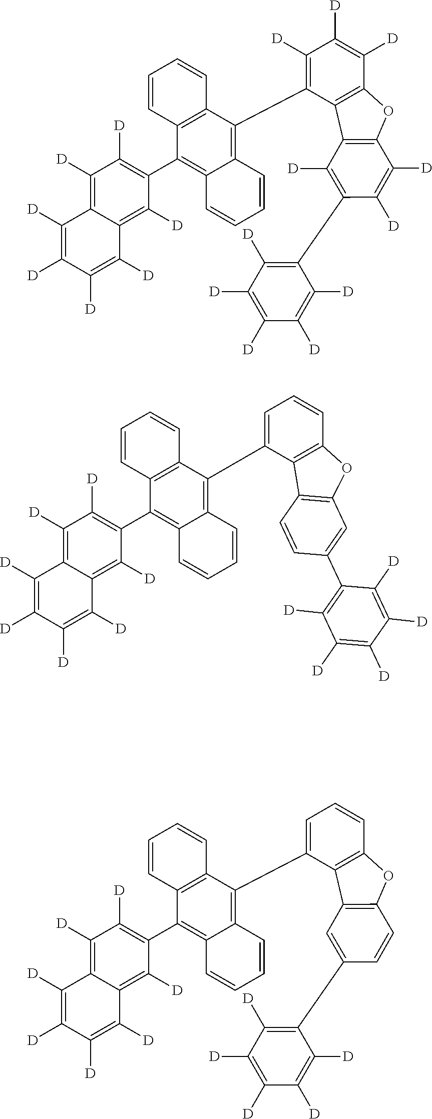 [SMILES: [2H]c1c([2H])c([2H])c(-c2c([2H])c([2H])c3oc4c([2H])c([2H])c([2H])c(-c5c6ccccc6c(-c6c([2H])c([2H])c7c([2H])c([2H])c([2H])c([2H])c7c6[2H])c6ccccc56)c4c3c2[2H])c([2H])c1[2H] ; [2H]c1c([2H])c([2H])c(-c2ccc3c(c2)oc2cccc(-c4c5ccccc5c(-c5c([2H])c([2H])c6c([2H])c([2H])c([2H])c([2H])c6c5[2H])c5ccccc45)c23)c([2H])c1[2H] ; [2H]c1c([2H])c([2H])c(-c2ccc3oc4cccc(-c5c6ccccc6c(-c6c([2H])c([2H])c7c([2H])c([2H])c([2H])c([2H])c7c6[2H])c6ccccc56)c4c3c2)c([2H])c1[2H]]

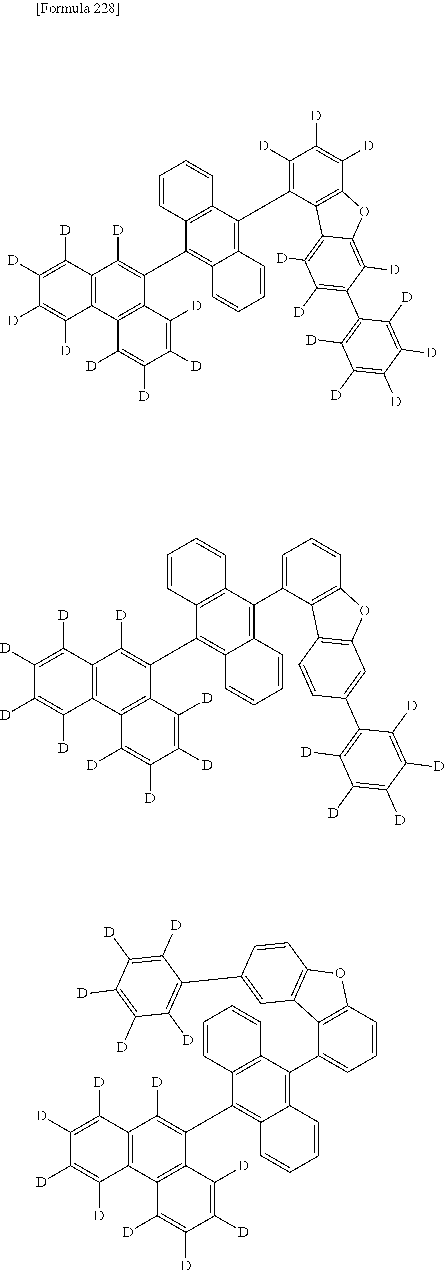

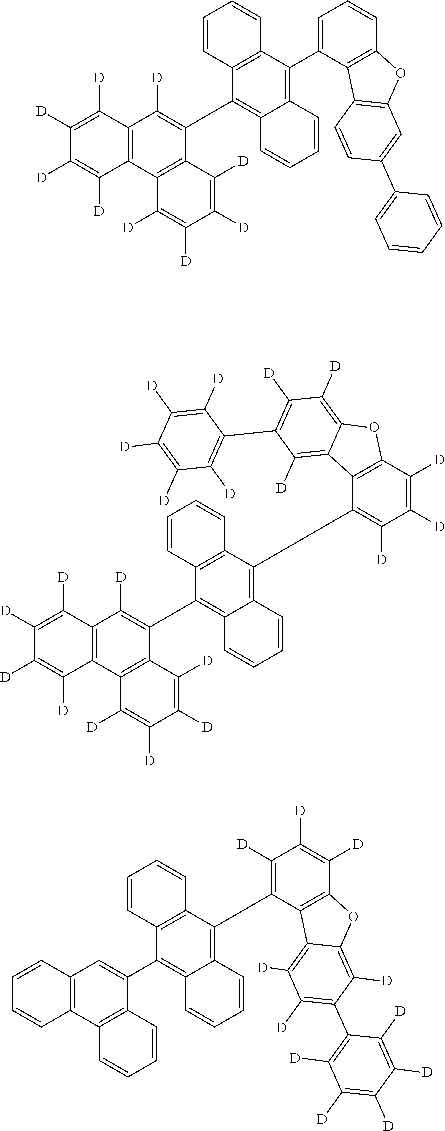 [SMILES: [2H]c1c([2H])c([2H])c2c(c1[2H])c([2H])c(-c1c3ccccc3c(-c3cccc4oc5cc(-c6ccccc6)ccc5c34)c3ccccc13)c1c([2H])c([2H])c([2H])c([2H])c12 ; [2H]c1c([2H])c([2H])c(-c2c([2H])c([2H])c3oc4c([2H])c([2H])c([2H])c(-c5c6ccccc6c(-c6c([2H])c7c([2H])c([2H])c([2H])c([2H])c7c7c([2H])c([2H])c([2H])c([2H])c67)c6ccccc56)c4c3c2[2H])c([2H])c1[2H] ; [2H]c1c([2H])c([2H])c(-c2c([2H])c([2H])c3c(oc4c([2H])c([2H])c([2H])c(-c5c6ccccc6c(-c6cc7ccccc7c7ccccc67)c6ccccc56)c43)c2[2H])c([2H])c1[2H]]

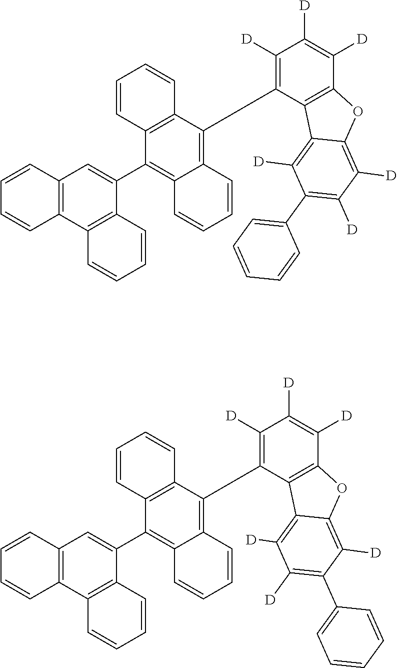 [SMILES: [2H]c1c(-c2ccccc2)c([2H])c2c(oc3c([2H])c([2H])c([2H])c(-c4c5ccccc5c(-c5cc6ccccc6c6ccccc56)c5ccccc45)c32)c1[2H] ; [2H]c1c([2H])c(-c2c3ccccc3c(-c3cc4ccccc4c4ccccc34)c3ccccc23)c2c(oc3c([2H])c(-c4ccccc4)c([2H])c([2H])c32)c1[2H]]

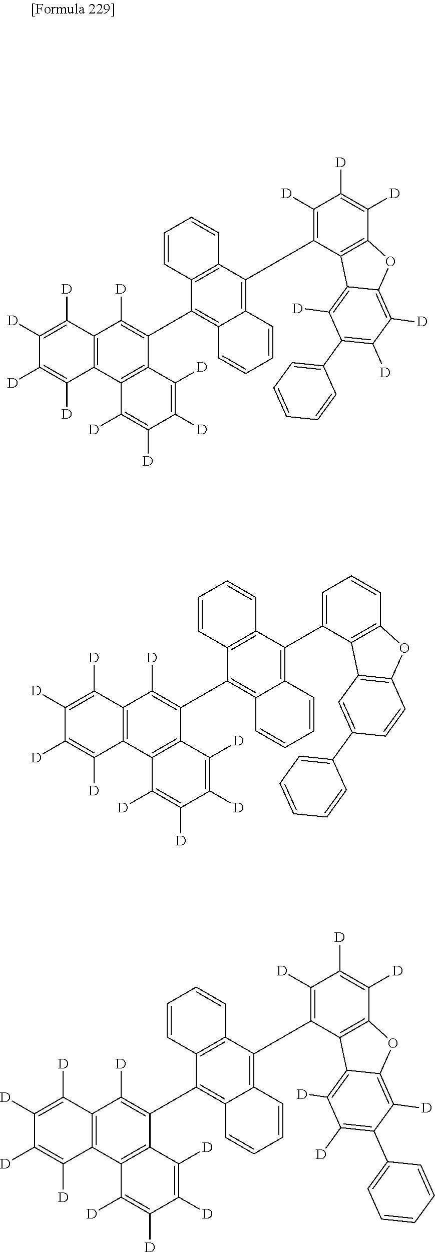 [SMILES: [2H]c1c(-c2ccccc2)c([2H])c2c(oc3c([2H])c([2H])c([2H])c(-c4c5ccccc5c(-c5c([2H])c6c([2H])c([2H])c([2H])c([2H])c6c6c([2H])c([2H])c([2H])c([2H])c56)c5ccccc45)c32)c1[2H] ; [2H]c1c([2H])c([2H])c2c(c1[2H])c([2H])c(-c1c3ccccc3c(-c3cccc4oc5ccc(-c6ccccc6)cc5c34)c3ccccc13)c1c([2H])c([2H])c([2H])c([2H])c12 ; [2H]c1c([2H])c(-c2c3ccccc3c(-c3c([2H])c4c([2H])c([2H])c([2H])c([2H])c4c4c([2H])c([2H])c([2H])c([2H])c34)c3ccccc23)c2c(oc3c([2H])c(-c4ccccc4)c([2H])c([2H])c32)c1[2H]]

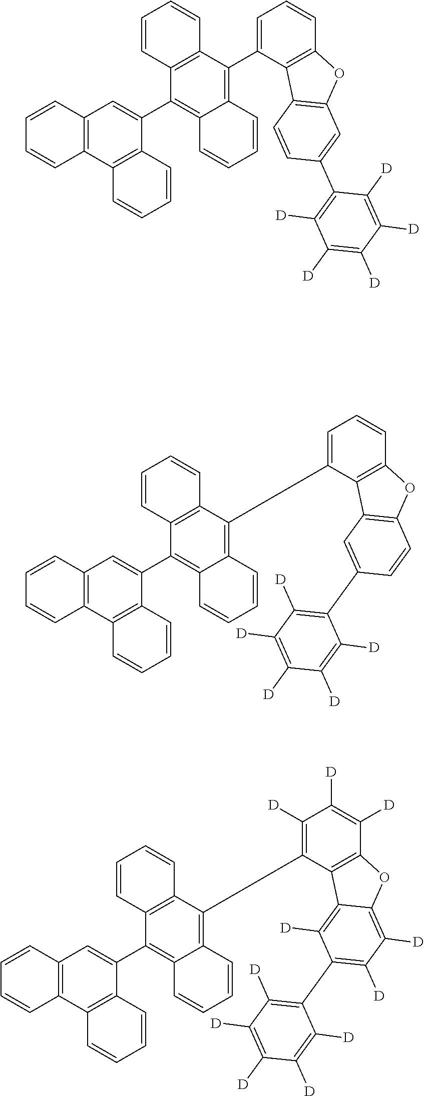 [SMILES: [2H]c1c([2H])c([2H])c(-c2ccc3c(c2)oc2cccc(-c4c5ccccc5c(-c5cc6ccccc6c6ccccc56)c5ccccc45)c23)c([2H])c1[2H] ; [2H]c1c([2H])c([2H])c(-c2ccc3oc4cccc(-c5c6ccccc6c(-c6cc7ccccc7c7ccccc67)c6ccccc56)c4c3c2)c([2H])c1[2H] ; [2H]c1c([2H])c([2H])c(-c2c([2H])c([2H])c3oc4c([2H])c([2H])c([2H])c(-c5c6ccccc6c(-c6cc7ccccc7c7ccccc67)c6ccccc56)c4c3c2[2H])c([2H])c1[2H]]

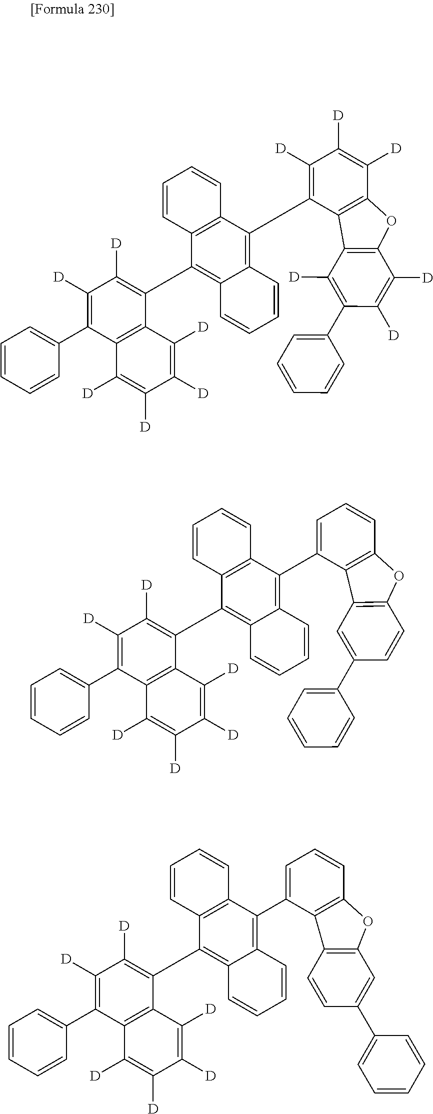 [SMILES: [2H]c1c(-c2ccccc2)c([2H])c2c(oc3c([2H])c([2H])c([2H])c(-c4c5ccccc5c(-c5c([2H])c([2H])c(-c6ccccc6)c6c([2H])c([2H])c([2H])c([2H])c56)c5ccccc45)c32)c1[2H] ; [2H]c1c([2H])c([2H])c2c(-c3c4ccccc4c(-c4cccc5oc6ccc(-c7ccccc7)cc6c45)c4ccccc34)c([2H])c([2H])c(-c3ccccc3)c2c1[2H] ; [2H]c1c([2H])c([2H])c2c(-c3c4ccccc4c(-c4cccc5oc6cc(-c7ccccc7)ccc6c45)c4ccccc34)c([2H])c([2H])c(-c3ccccc3)c2c1[2H]]

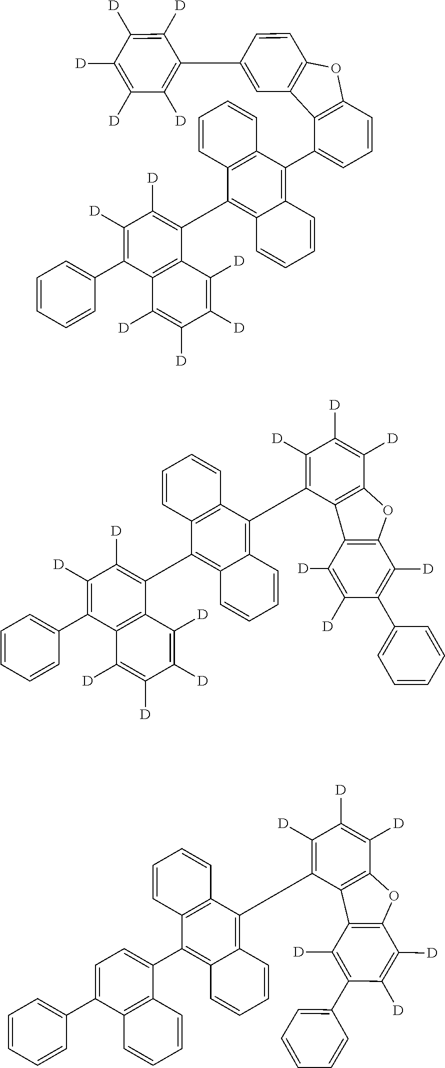 [SMILES: [2H]c1c([2H])c([2H])c(-c2ccc3oc4cccc(-c5c6ccccc6c(-c6c([2H])c([2H])c(-c7ccccc7)c7c([2H])c([2H])c([2H])c([2H])c67)c6ccccc56)c4c3c2)c([2H])c1[2H] ; [2H]c1c([2H])c(-c2c3ccccc3c(-c3c([2H])c([2H])c(-c4ccccc4)c4c([2H])c([2H])c([2H])c([2H])c34)c3ccccc23)c2c(oc3c([2H])c(-c4ccccc4)c([2H])c([2H])c32)c1[2H] ; [2H]c1c(-c2ccccc2)c([2H])c2c(oc3c([2H])c([2H])c([2H])c(-c4c5ccccc5c(-c5ccc(-c6ccccc6)c6ccccc56)c5ccccc45)c32)c1[2H]]

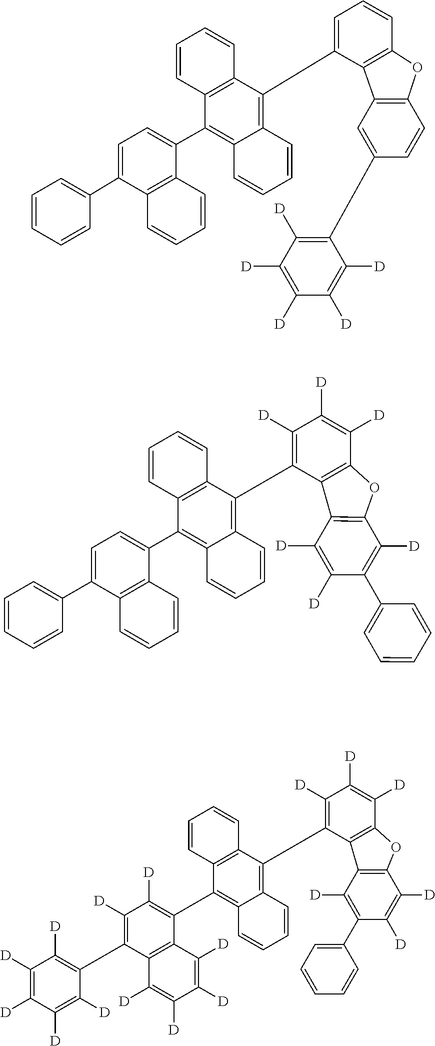 [SMILES: [2H]c1c([2H])c([2H])c(-c2ccc3oc4cccc(-c5c6ccccc6c(-c6ccc(-c7ccccc7)c7ccccc67)c6ccccc56)c4c3c2)c([2H])c1[2H] ; [2H]c1c([2H])c(-c2c3ccccc3c(-c3ccc(-c4ccccc4)c4ccccc34)c3ccccc23)c2c(oc3c([2H])c(-c4ccccc4)c([2H])c([2H])c32)c1[2H] ; [2H]c1c([2H])c([2H])c(-c2c([2H])c([2H])c(-c3c4ccccc4c(-c4c([2H])c([2H])c([2H])c5oc6c([2H])c([2H])c(-c7ccccc7)c([2H])c6c45)c4ccccc34)c3c([2H])c([2H])c([2H])c([2H])c23)c([2H])c1[2H]]

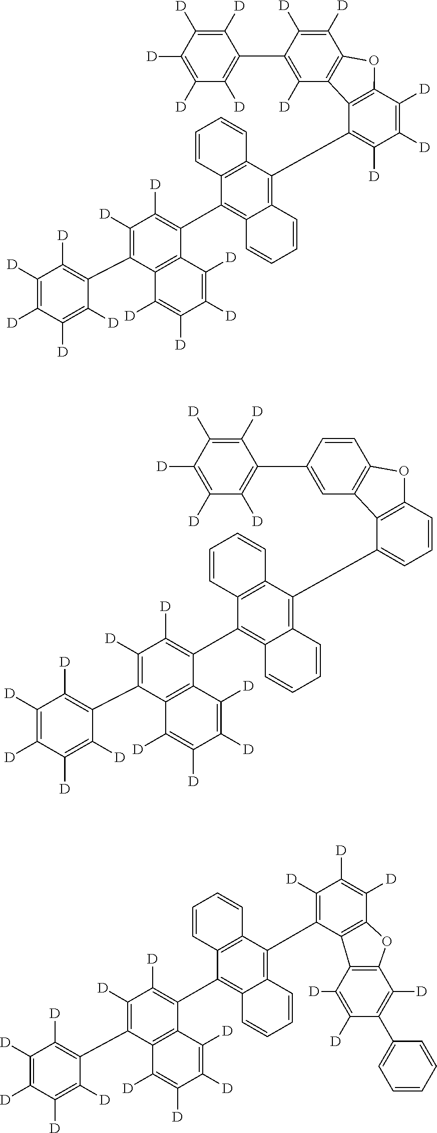

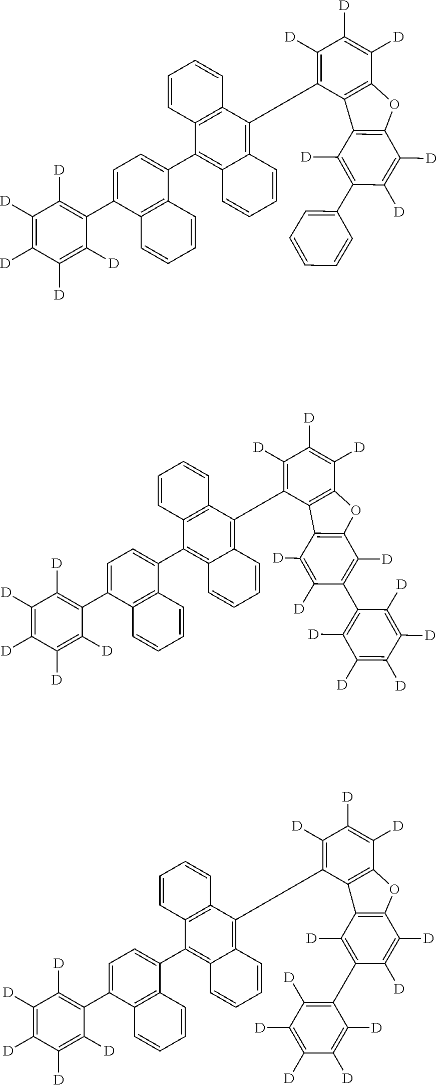 [SMILES: [2H]c1c([2H])c([2H])c(-c2ccc(-c3c4ccccc4c(-c4c([2H])c([2H])c([2H])c5oc6c([2H])c([2H])c(-c7ccccc7)c([2H])c6c45)c4ccccc34)c3ccccc23)c([2H])c1[2H] ; [2H]c1c([2H])c([2H])c(-c2c([2H])c([2H])c3c(oc4c([2H])c([2H])c([2H])c(-c5c6ccccc6c(-c6ccc(-c7c([2H])c([2H])c([2H])c([2H])c7[2H])c7ccccc67)c6ccccc56)c43)c2[2H])c([2H])c1[2H] ; [2H]c1c([2H])c([2H])c(-c2c([2H])c([2H])c3oc4c([2H])c([2H])c([2H])c(-c5c6ccccc6c(-c6ccc(-c7c([2H])c([2H])c([2H])c([2H])c7[2H])c7ccccc67)c6ccccc56)c4c3c2[2H])c([2H])c1[2H]]

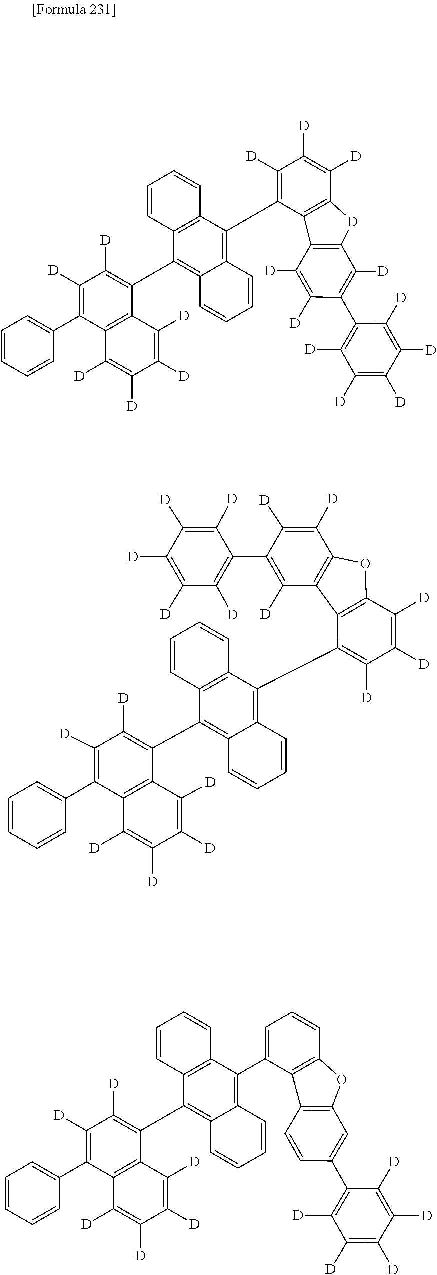

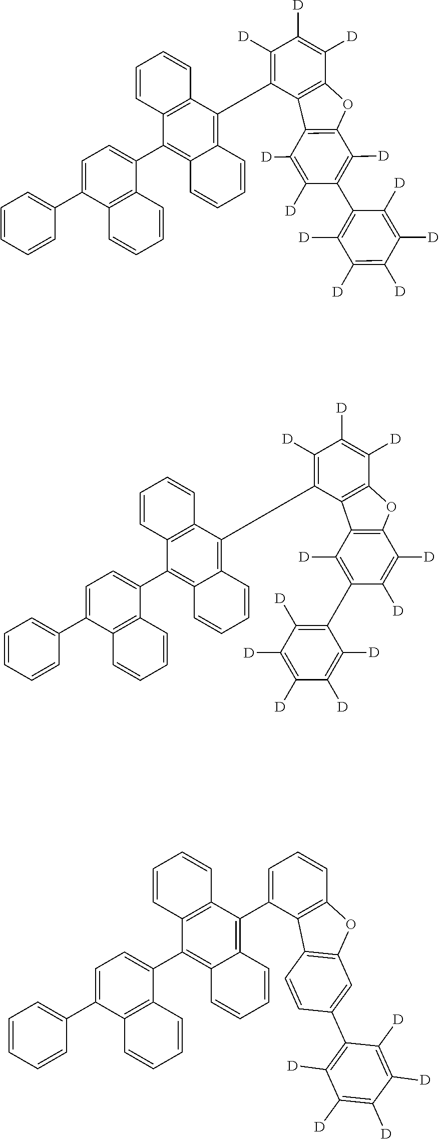 [SMILES: [2H]c1c([2H])c([2H])c(-c2c([2H])c([2H])c3c(oc4c([2H])c([2H])c([2H])c(-c5c6ccccc6c(-c6ccc(-c7ccccc7)c7ccccc67)c6ccccc56)c43)c2[2H])c([2H])c1[2H] ; [2H]c1c([2H])c([2H])c(-c2c([2H])c([2H])c3oc4c([2H])c([2H])c([2H])c(-c5c6ccccc6c(-c6ccc(-c7ccccc7)c7ccccc67)c6ccccc56)c4c3c2[2H])c([2H])c1[2H] ; [2H]c1c([2H])c([2H])c(-c2ccc3c(c2)oc2cccc(-c4c5ccccc5c(-c5ccc(-c6ccccc6)c6ccccc56)c5ccccc45)c23)c([2H])c1[2H]]

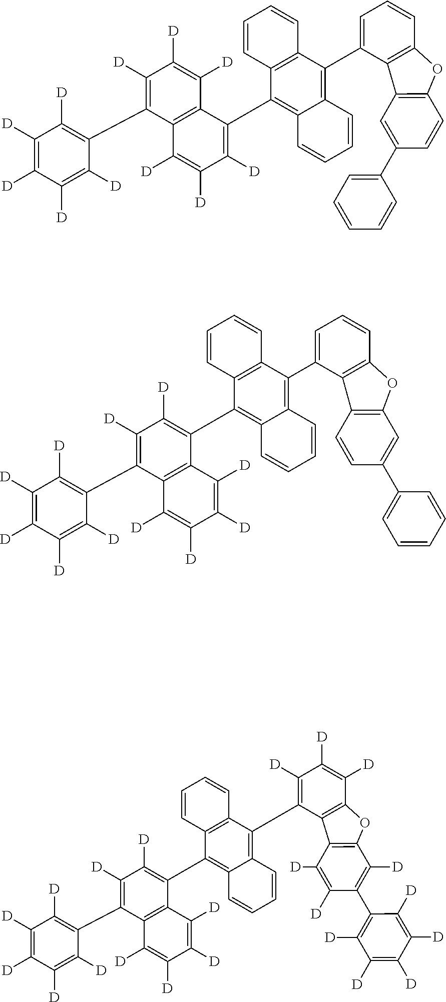 [SMILES: [2H]c1c([2H])c([2H])c(-c2c([2H])c([2H])c([2H])c3c(-c4c5ccccc5c(-c5cccc6oc7ccc(-c8ccccc8)cc7c56)c5ccccc45)c([2H])c([2H])c([2H])c23)c([2H])c1[2H] ; [2H]c1c([2H])c([2H])c(-c2c([2H])c([2H])c(-c3c4ccccc4c(-c4cccc5oc6cc(-c7ccccc7)ccc6c45)c4ccccc34)c3c([2H])c([2H])c([2H])c([2H])c23)c([2H])c1[2H] ; [2H]c1c([2H])c([2H])c(-c2c([2H])c([2H])c3c(oc4c([2H])c([2H])c([2H])c(-c5c6ccccc6c(-c6c([2H])c([2H])c(-c7c([2H])c([2H])c([2H])c([2H])c7[2H])c7c([2H])c([2H])c([2H])c([2H])c67)c6ccccc56)c43)c2[2H])c([2H])c1[2H]]

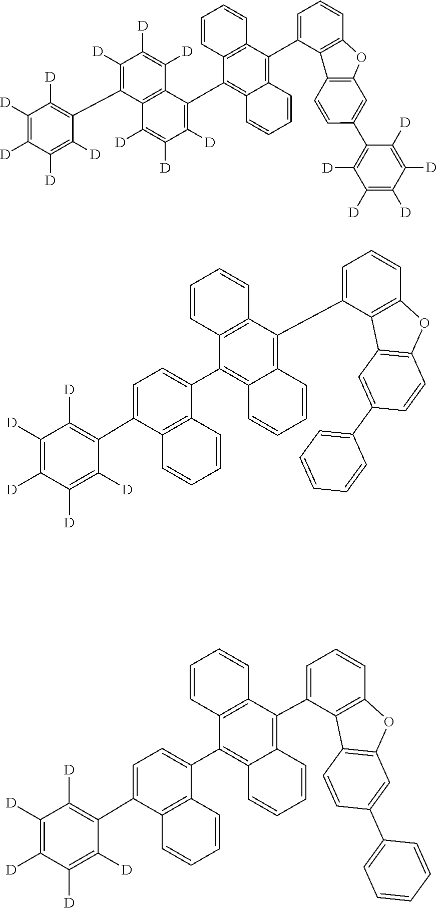

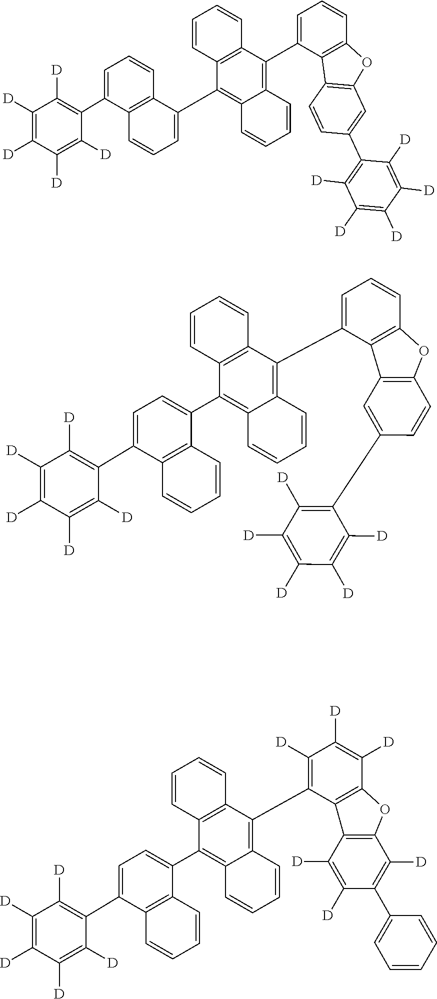 [SMILES: [2H]c1c([2H])c([2H])c(-c2ccc3c(c2)oc2cccc(-c4c5ccccc5c(-c5cccc6c(-c7c([2H])c([2H])c([2H])c([2H])c7[2H])cccc56)c5ccccc45)c23)c([2H])c1[2H] ; [2H]c1c([2H])c([2H])c(-c2ccc3oc4cccc(-c5c6ccccc6c(-c6ccc(-c7c([2H])c([2H])c([2H])c([2H])c7[2H])c7ccccc67)c6ccccc56)c4c3c2)c([2H])c1[2H] ; [2H]c1c([2H])c([2H])c(-c2ccc(-c3c4ccccc4c(-c4c([2H])c([2H])c([2H])c5oc6c([2H])c(-c7ccccc7)c([2H])c([2H])c6c45)c4ccccc34)c3ccccc23)c([2H])c1[2H]]

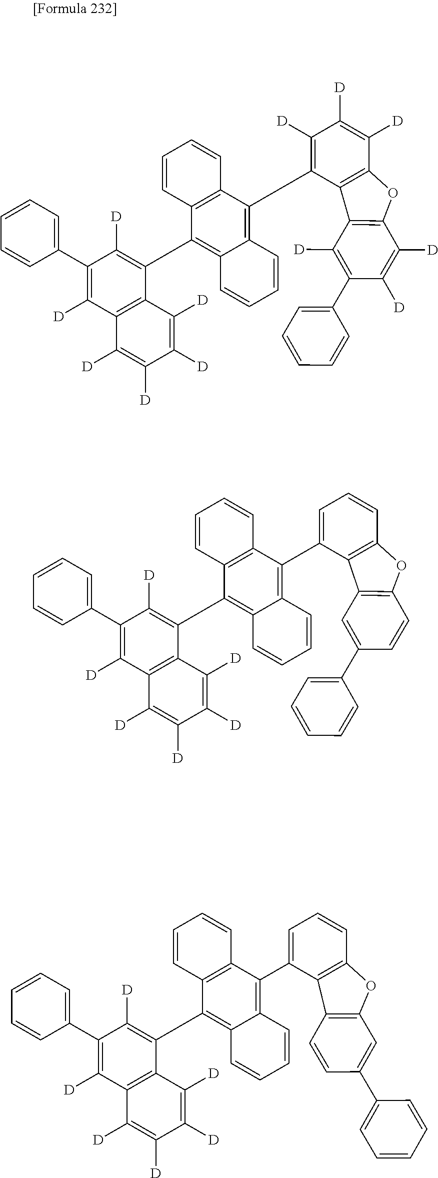 [SMILES: [2H]c1c(-c2ccccc2)c([2H])c2c(oc3c([2H])c([2H])c([2H])c(-c4c5ccccc5c(-c5c([2H])c(-c6ccccc6)c([2H])c6c([2H])c([2H])c([2H])c([2H])c56)c5ccccc45)c32)c1[2H] ; [2H]c1c([2H])c([2H])c2c(-c3c4ccccc4c(-c4cccc5oc6ccc(-c7ccccc7)cc6c45)c4ccccc34)c([2H])c(-c3ccccc3)c([2H])c2c1[2H] ; [2H]c1c([2H])c([2H])c2c(-c3c4ccccc4c(-c4cccc5oc6cc(-c7ccccc7)ccc6c45)c4ccccc34)c([2H])c(-c3ccccc3)c([2H])c2c1[2H]]

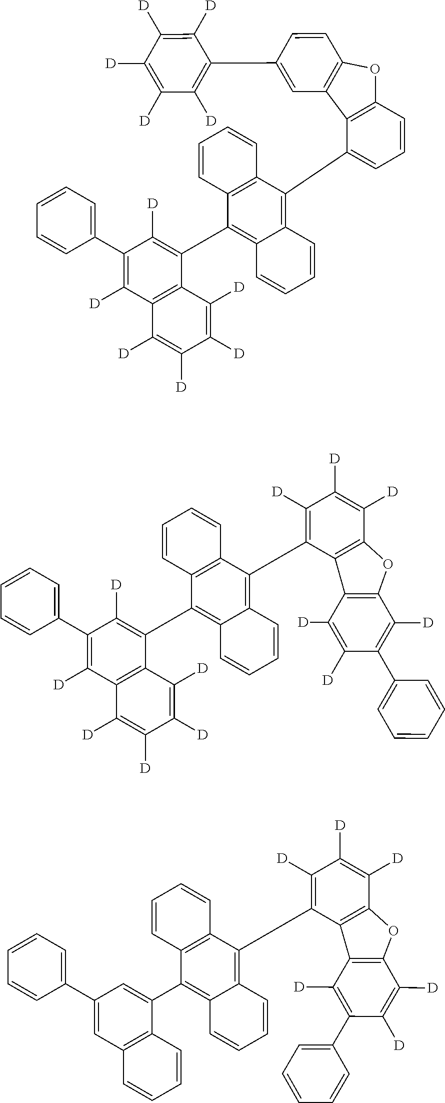 [SMILES: [2H]c1c([2H])c([2H])c(-c2ccc3oc4cccc(-c5c6ccccc6c(-c6c([2H])c(-c7ccccc7)c([2H])c7c([2H])c([2H])c([2H])c([2H])c67)c6ccccc56)c4c3c2)c([2H])c1[2H] ; [2H]c1c([2H])c(-c2c3ccccc3c(-c3c([2H])c(-c4ccccc4)c([2H])c4c([2H])c([2H])c([2H])c([2H])c34)c3ccccc23)c2c(oc3c([2H])c(-c4ccccc4)c([2H])c([2H])c32)c1[2H] ; [2H]c1c(-c2ccccc2)c([2H])c2c(oc3c([2H])c([2H])c([2H])c(-c4c5ccccc5c(-c5cc(-c6ccccc6)cc6ccccc56)c5ccccc45)c32)c1[2H]]

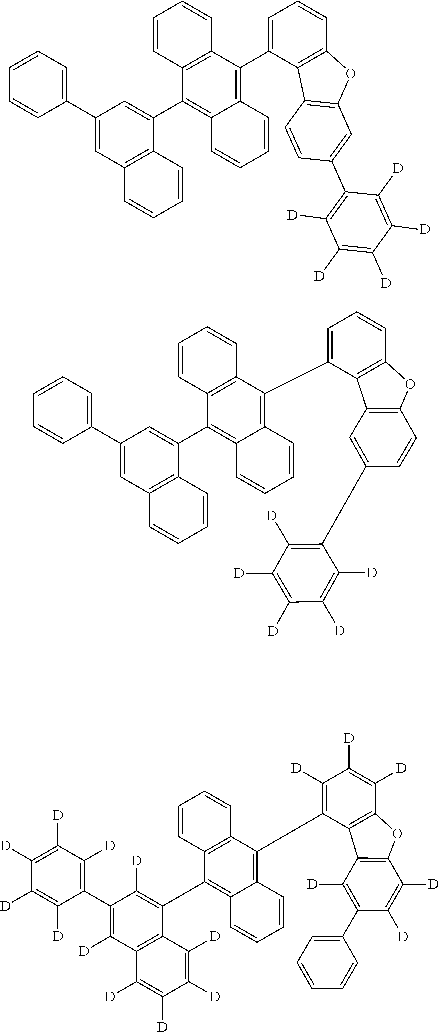

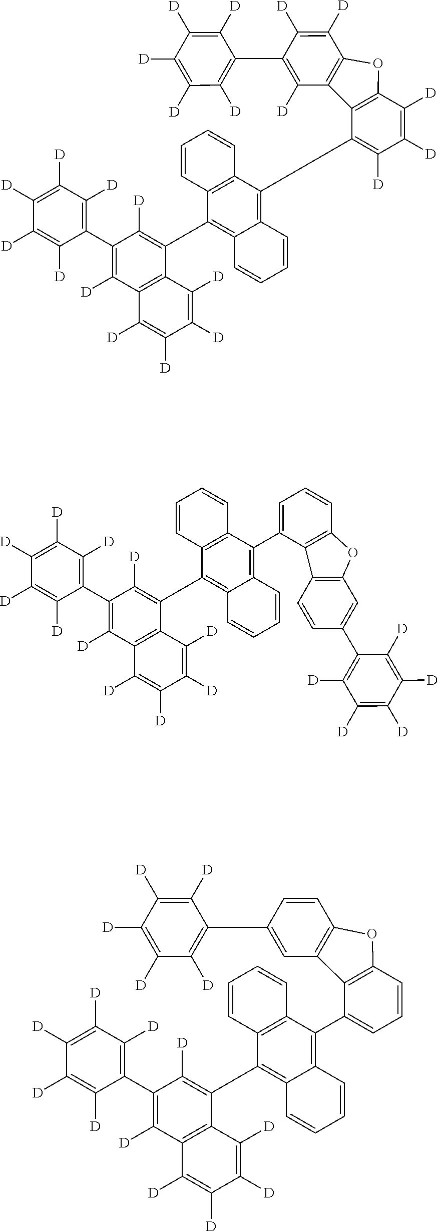

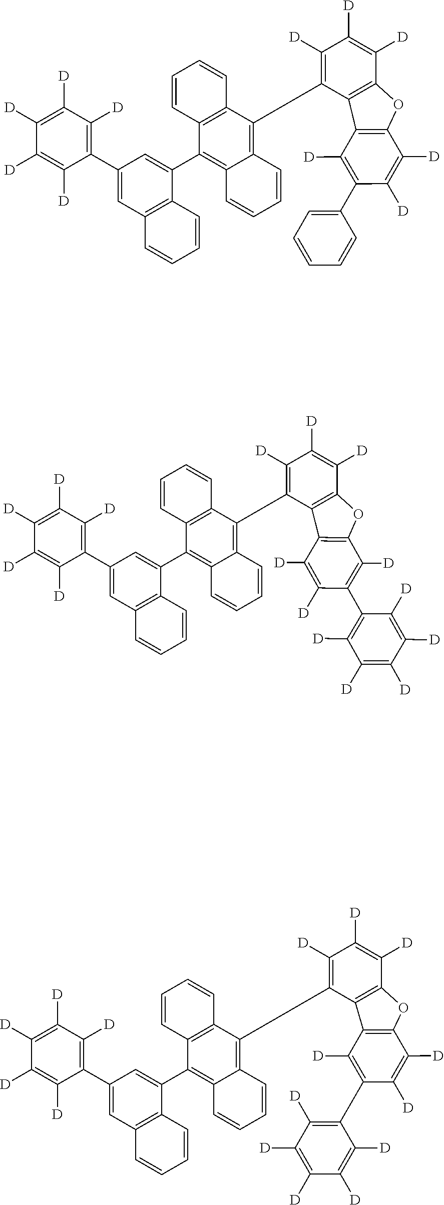 [SMILES: [2H]c1c([2H])c([2H])c(-c2cc(-c3c4ccccc4c(-c4c([2H])c([2H])c([2H])c5oc6c([2H])c([2H])c(-c7ccccc7)c([2H])c6c45)c4ccccc34)c3ccccc3c2)c([2H])c1[2H] ; [2H]c1c([2H])c([2H])c(-c2cc(-c3c4ccccc4c(-c4c([2H])c([2H])c([2H])c5oc6c([2H])c(-c7c([2H])c([2H])c([2H])c([2H])c7[2H])c([2H])c([2H])c6c45)c4ccccc34)c3ccccc3c2)c([2H])c1[2H] ; [2H]c1c([2H])c([2H])c(-c2cc(-c3c4ccccc4c(-c4c([2H])c([2H])c([2H])c5oc6c([2H])c([2H])c(-c7c([2H])c([2H])c([2H])c([2H])c7[2H])c([2H])c6c45)c4ccccc34)c3ccccc3c2)c([2H])c1[2H]]

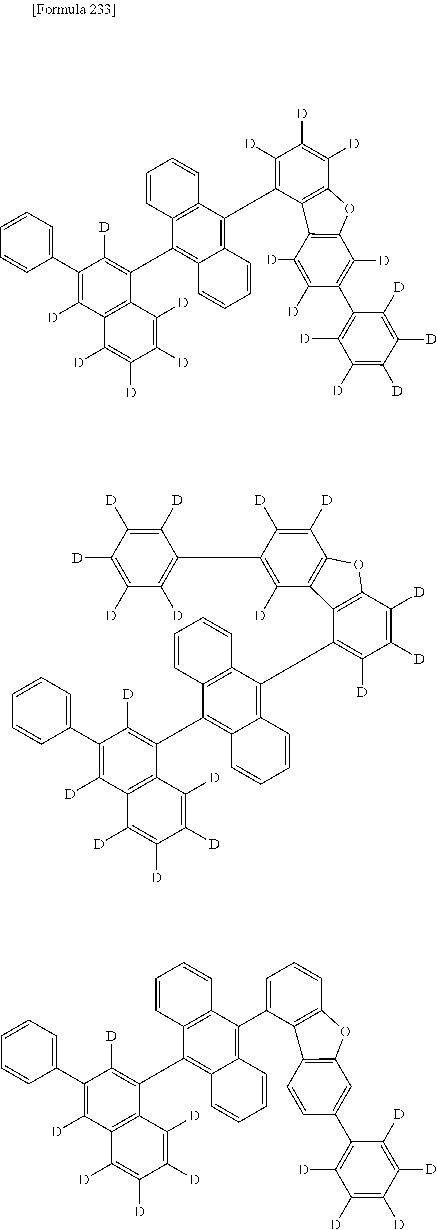

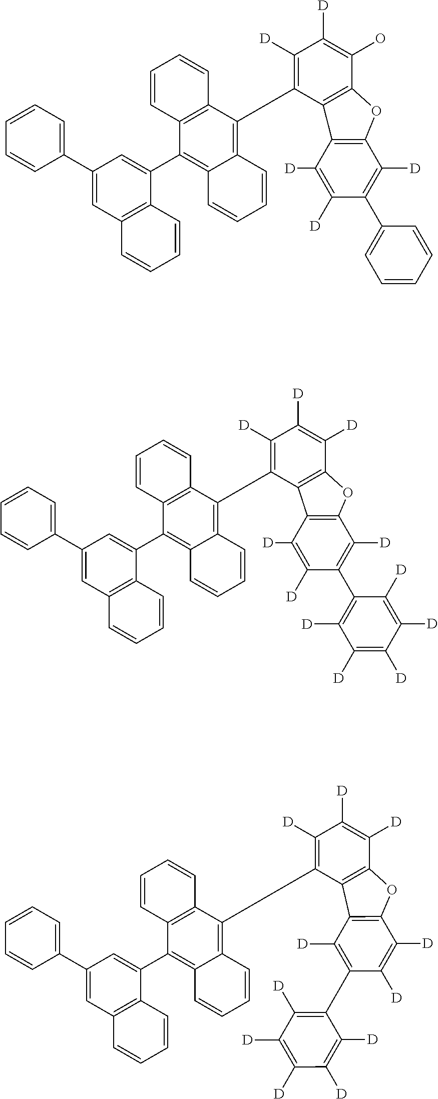

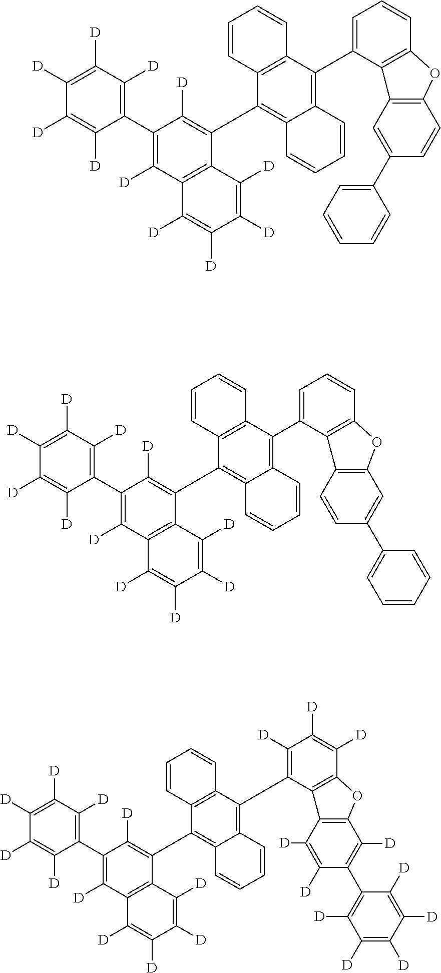 [SMILES: [2H]c1c([2H])c([2H])c(-c2c([2H])c(-c3c4ccccc4c(-c4cccc5oc6ccc(-c7ccccc7)cc6c45)c4ccccc34)c3c([2H])c([2H])c([2H])c([2H])c3c2[2H])c([2H])c1[2H] ; [2H]c1c([2H])c([2H])c(-c2c([2H])c(-c3c4ccccc4c(-c4cccc5oc6cc(-c7ccccc7)ccc6c45)c4ccccc34)c3c([2H])c([2H])c([2H])c([2H])c3c2[2H])c([2H])c1[2H] ; [2H]c1c([2H])c([2H])c(-c2c([2H])c([2H])c3c(oc4c([2H])c([2H])c([2H])c(-c5c6ccccc6c(-c6c([2H])c(-c7c([2H])c([2H])c([2H])c([2H])c7[2H])c([2H])c7c([2H])c([2H])c([2H])c([2H])c67)c6ccccc56)c43)c2[2H])c([2H])c1[2H]]

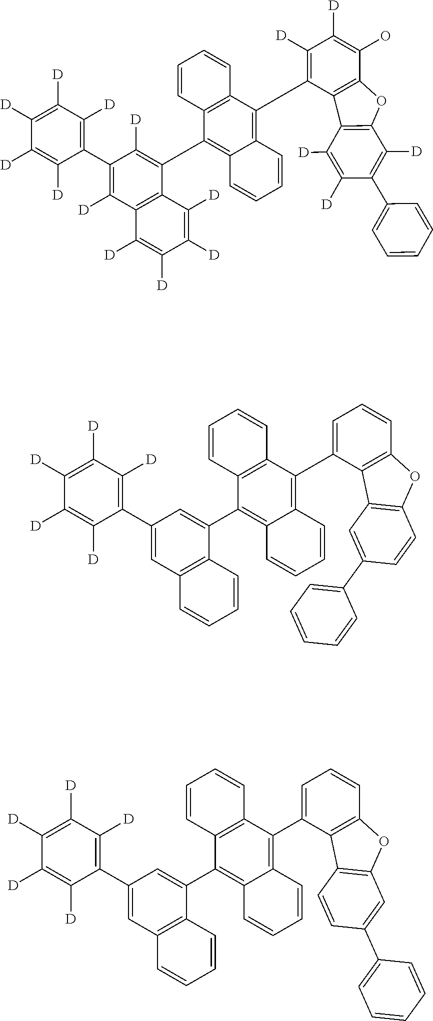

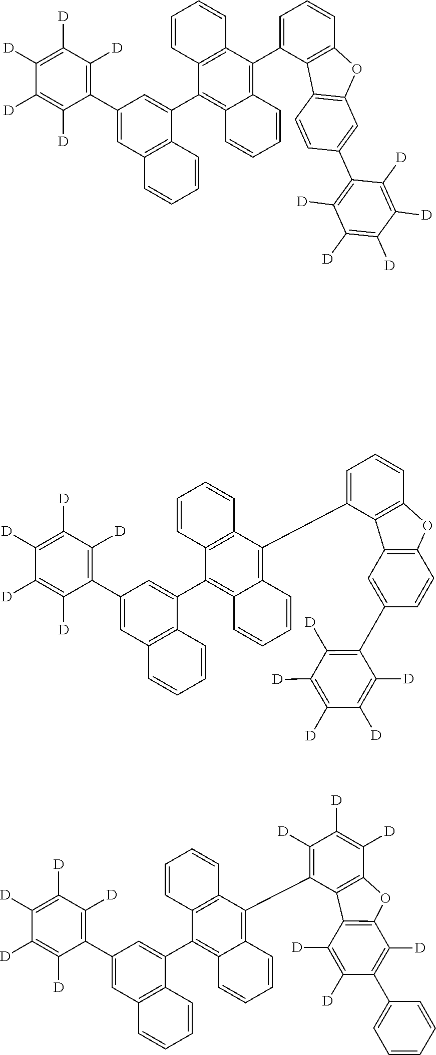 [SMILES: [2H]c1c([2H])c([2H])c(-c2cc(-c3c4ccccc4c(-c4cccc5oc6cc(-c7c([2H])c([2H])c([2H])c([2H])c7[2H])ccc6c45)c4ccccc34)c3ccccc3c2)c([2H])c1[2H] ; [2H]c1c([2H])c([2H])c(-c2cc(-c3c4ccccc4c(-c4cccc5oc6ccc(-c7c([2H])c([2H])c([2H])c([2H])c7[2H])cc6c45)c4ccccc34)c3ccccc3c2)c([2H])c1[2H] ; [2H]c1c([2H])c([2H])c(-c2cc(-c3c4ccccc4c(-c4c([2H])c([2H])c([2H])c5oc6c([2H])c(-c7ccccc7)c([2H])c([2H])c6c45)c4ccccc34)c3ccccc3c2)c([2H])c1[2H]]

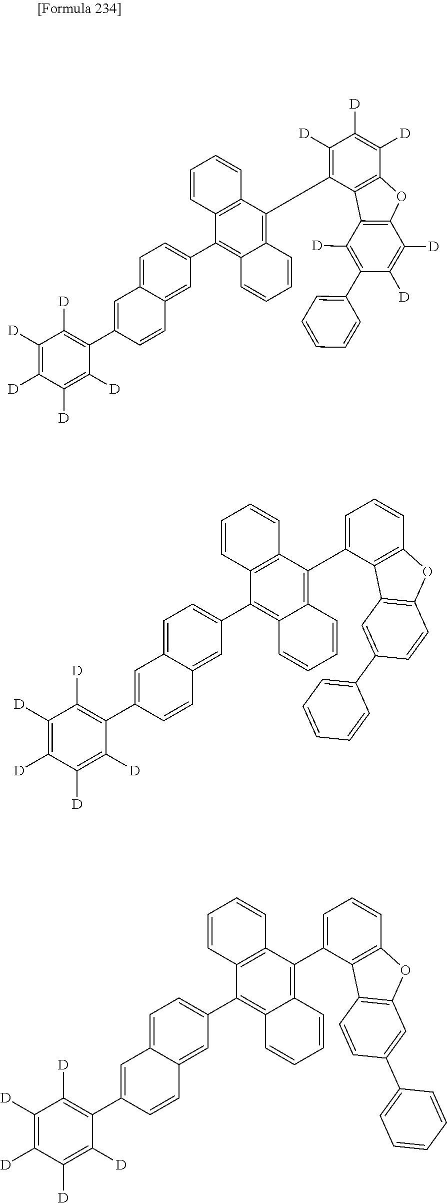 [SMILES: [2H]c1c([2H])c([2H])c(-c2ccc3cc(-c4c5ccccc5c(-c5c([2H])c([2H])c([2H])c6oc7c([2H])c([2H])c(-c8ccccc8)c([2H])c7c56)c5ccccc45)ccc3c2)c([2H])c1[2H] ; [2H]c1c([2H])c([2H])c(-c2ccc3cc(-c4c5ccccc5c(-c5cccc6oc7ccc(-c8ccccc8)cc7c56)c5ccccc45)ccc3c2)c([2H])c1[2H] ; [2H]c1c([2H])c([2H])c(-c2ccc3cc(-c4c5ccccc5c(-c5cccc6oc7cc(-c8ccccc8)ccc7c56)c5ccccc45)ccc3c2)c([2H])c1[2H]]

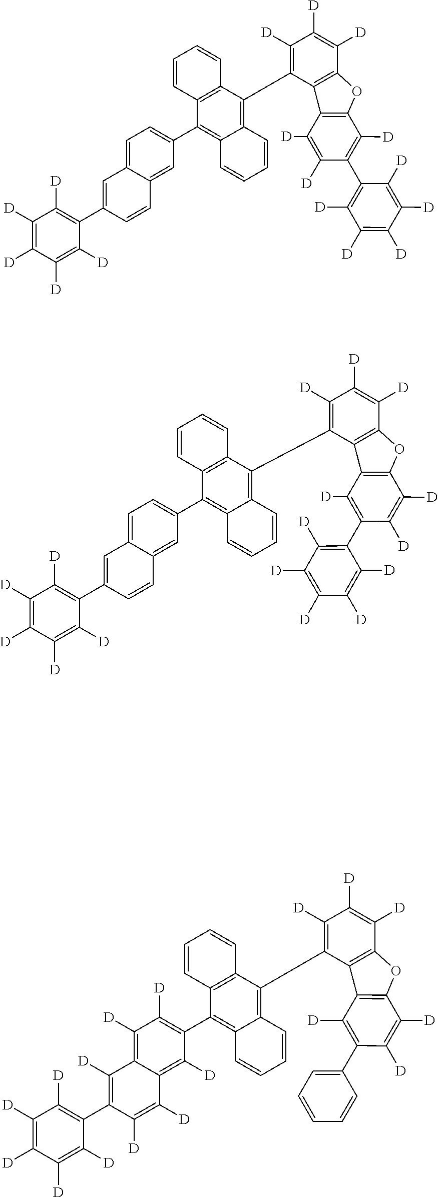

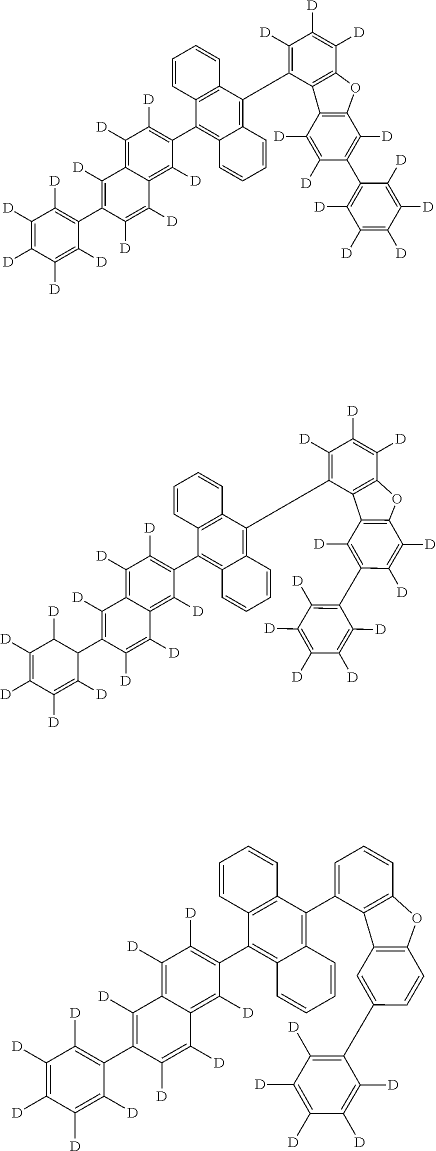

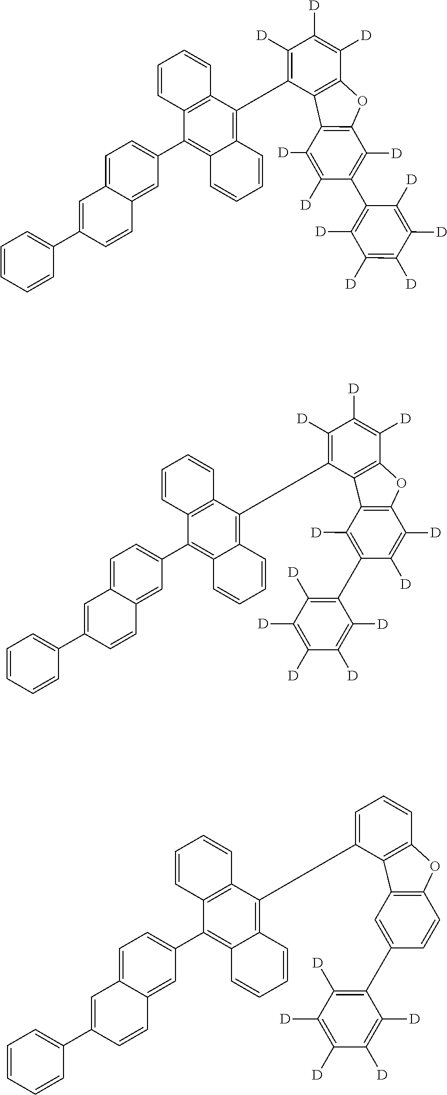 [SMILES: [2H]c1c([2H])c([2H])c(-c2c([2H])c([2H])c3c(oc4c([2H])c([2H])c([2H])c(-c5c6ccccc6c(-c6ccc7cc(-c8ccccc8)ccc7c6)c6ccccc56)c43)c2[2H])c([2H])c1[2H] ; [2H]c1c([2H])c([2H])c(-c2c([2H])c([2H])c3oc4c([2H])c([2H])c([2H])c(-c5c6ccccc6c(-c6ccc7cc(-c8ccccc8)ccc7c6)c6ccccc56)c4c3c2[2H])c([2H])c1[2H] ; [2H]c1c([2H])c([2H])c(-c2ccc3oc4cccc(-c5c6ccccc6c(-c6ccc7cc(-c8ccccc8)ccc7c6)c6ccccc56)c4c3c2)c([2H])c1[2H]]

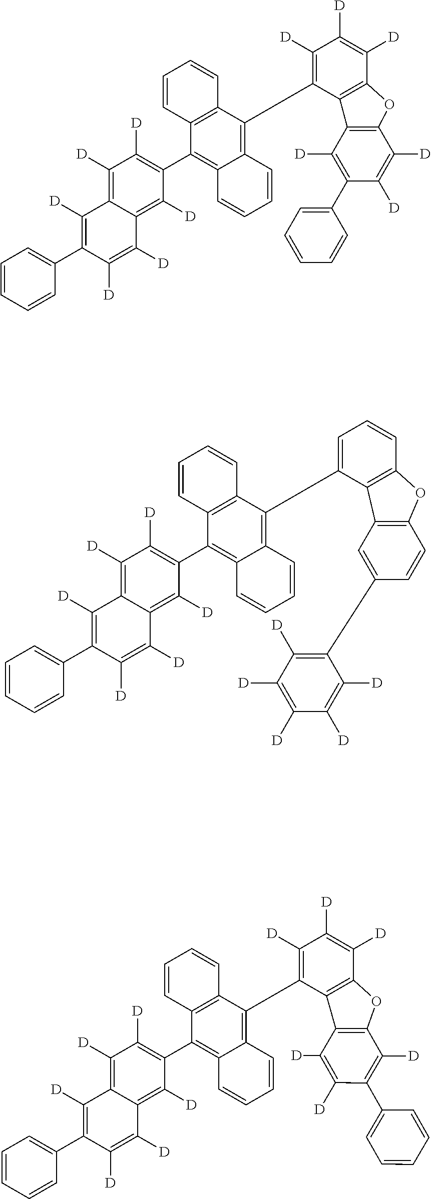 [SMILES: [2H]c1c(-c2ccccc2)c([2H])c2c(oc3c([2H])c([2H])c([2H])c(-c4c5ccccc5c(-c5c([2H])c([2H])c6c([2H])c(-c7ccccc7)c([2H])c([2H])c6c5[2H])c5ccccc45)c32)c1[2H] ; [2H]c1c([2H])c([2H])c(-c2ccc3oc4cccc(-c5c6ccccc6c(-c6c([2H])c([2H])c7c([2H])c(-c8ccccc8)c([2H])c([2H])c7c6[2H])c6ccccc56)c4c3c2)c([2H])c1[2H] ; [2H]c1c([2H])c(-c2c3ccccc3c(-c3c([2H])c([2H])c4c([2H])c(-c5ccccc5)c([2H])c([2H])c4c3[2H])c3ccccc23)c2c(oc3c([2H])c(-c4ccccc4)c([2H])c([2H])c32)c1[2H]]

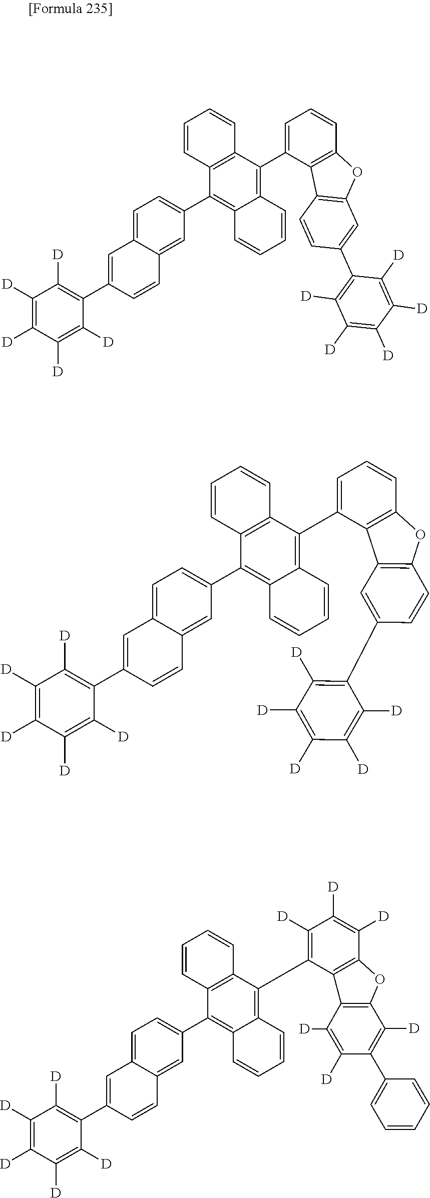 [SMILES: [2H]c1c([2H])c([2H])c(-c2ccc3cc(-c4c5ccccc5c(-c5cccc6oc7cc(-c8c([2H])c([2H])c([2H])c([2H])c8[2H])ccc7c56)c5ccccc45)ccc3c2)c([2H])c1[2H] ; [2H]c1c([2H])c([2H])c(-c2ccc3cc(-c4c5ccccc5c(-c5cccc6oc7ccc(-c8c([2H])c([2H])c([2H])c([2H])c8[2H])cc7c56)c5ccccc45)ccc3c2)c([2H])c1[2H] ; [2H]c1c([2H])c([2H])c(-c2ccc3cc(-c4c5ccccc5c(-c5c([2H])c([2H])c([2H])c6oc7c([2H])c(-c8ccccc8)c([2H])c([2H])c7c56)c5ccccc45)ccc3c2)c([2H])c1[2H]]

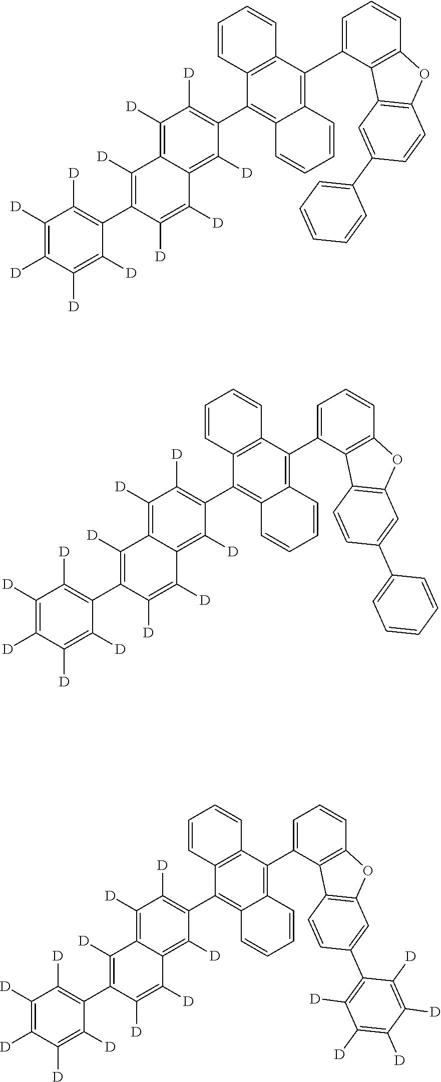 [SMILES: [2H]c1c([2H])c([2H])c(-c2c([2H])c([2H])c3c([2H])c(-c4c5ccccc5c(-c5cccc6oc7ccc(-c8ccccc8)cc7c56)c5ccccc45)c([2H])c([2H])c3c2[2H])c([2H])c1[2H] ; [2H]c1c([2H])c([2H])c(-c2c([2H])c([2H])c3c([2H])c(-c4c5ccccc5c(-c5cccc6oc7cc(-c8ccccc8)ccc7c56)c5ccccc45)c([2H])c([2H])c3c2[2H])c([2H])c1[2H] ; [2H]c1c([2H])c([2H])c(-c2ccc3c(c2)oc2cccc(-c4c5ccccc5c(-c5c([2H])c([2H])c6c([2H])c(-c7c([2H])c([2H])c([2H])c([2H])c7[2H])c([2H])c([2H])c6c5[2H])c5ccccc45)c23)c([2H])c1[2H]]

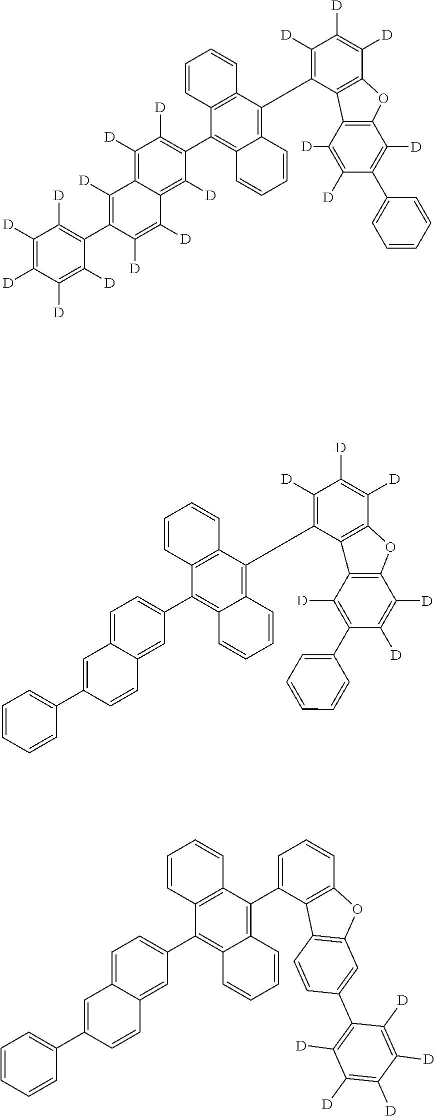 [SMILES: [2H]c1c([2H])c([2H])c(-c2c([2H])c([2H])c3c([2H])c(-c4c5ccccc5c(-c5c([2H])c([2H])c([2H])c6oc7c([2H])c(-c8ccccc8)c([2H])c([2H])c7c56)c5ccccc45)c([2H])c([2H])c3c2[2H])c([2H])c1[2H] ; [2H]c1c(-c2ccccc2)c([2H])c2c(oc3c([2H])c([2H])c([2H])c(-c4c5ccccc5c(-c5ccc6cc(-c7ccccc7)ccc6c5)c5ccccc45)c32)c1[2H] ; [2H]c1c([2H])c([2H])c(-c2ccc3c(c2)oc2cccc(-c4c5ccccc5c(-c5ccc6cc(-c7ccccc7)ccc6c5)c5ccccc45)c23)c([2H])c1[2H]]

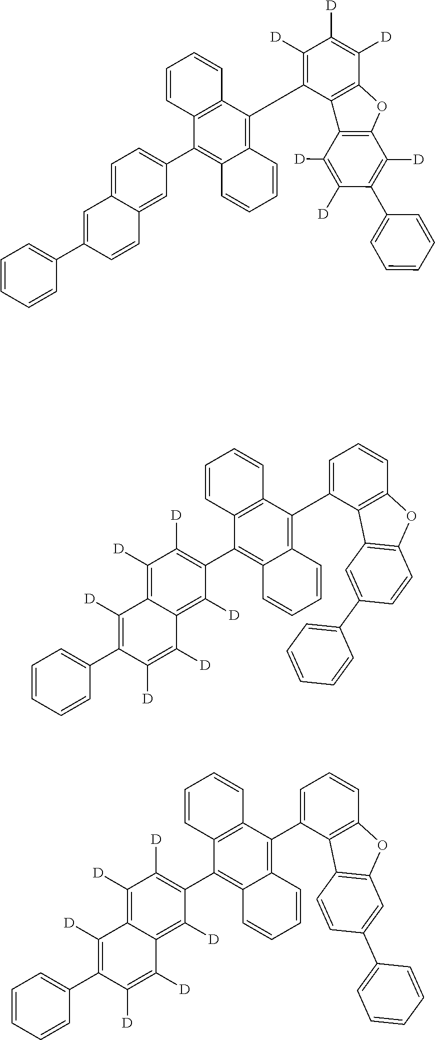 [SMILES: [2H]c1c([2H])c(-c2c3ccccc3c(-c3ccc4cc(-c5ccccc5)ccc4c3)c3ccccc23)c2c(oc3c([2H])c(-c4ccccc4)c([2H])c([2H])c32)c1[2H] ; [2H]c1c(-c2c3ccccc3c(-c3cccc4oc5ccc(-c6ccccc6)cc5c34)c3ccccc23)c([2H])c2c([2H])c([2H])c(-c3ccccc3)c([2H])c2c1[2H] ; [2H]c1c(-c2c3ccccc3c(-c3cccc4oc5cc(-c6ccccc6)ccc5c34)c3ccccc23)c([2H])c2c([2H])c([2H])c(-c3ccccc3)c([2H])c2c1[2H]]

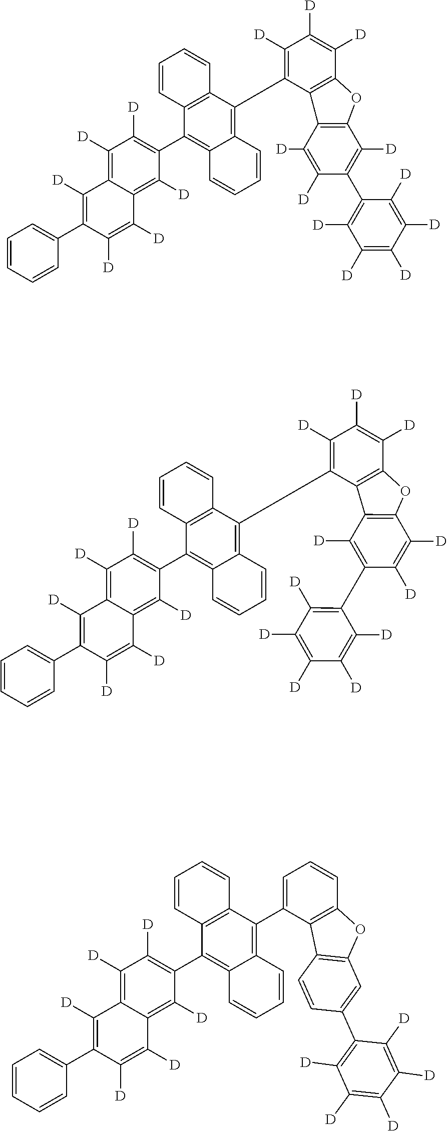

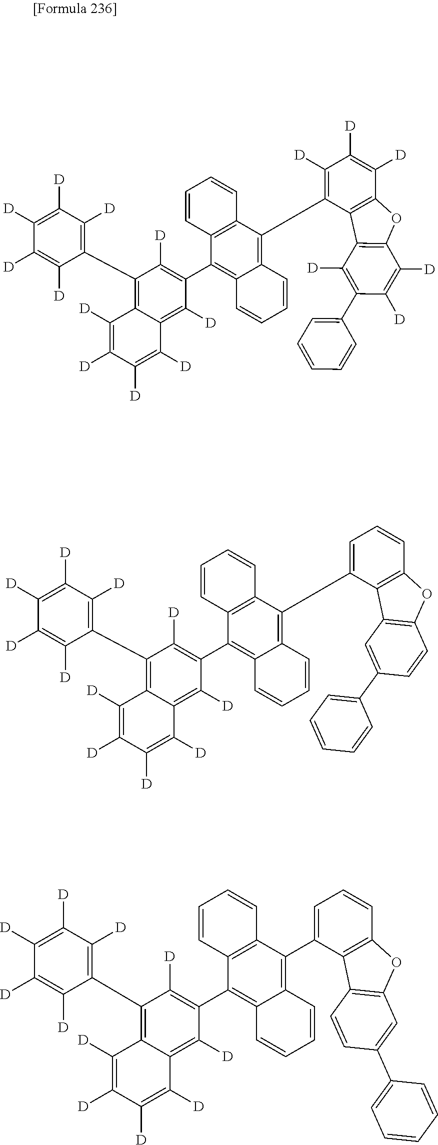 [SMILES: [2H]c1c([2H])c([2H])c(-c2c([2H])c(-c3c4ccccc4c(-c4c([2H])c([2H])c([2H])c5oc6c([2H])c([2H])c(-c7ccccc7)c([2H])c6c45)c4ccccc34)c([2H])c3c([2H])c([2H])c([2H])c([2H])c23)c([2H])c1[2H] ; [2H]c1c([2H])c([2H])c(-c2c([2H])c(-c3c4ccccc4c(-c4cccc5oc6ccc(-c7ccccc7)cc6c45)c4ccccc34)c([2H])c3c([2H])c([2H])c([2H])c([2H])c23)c([2H])c1[2H] ; [2H]c1c([2H])c([2H])c(-c2c([2H])c(-c3c4ccccc4c(-c4cccc5oc6cc(-c7ccccc7)ccc6c45)c4ccccc34)c([2H])c3c([2H])c([2H])c([2H])c([2H])c23)c([2H])c1[2H]]

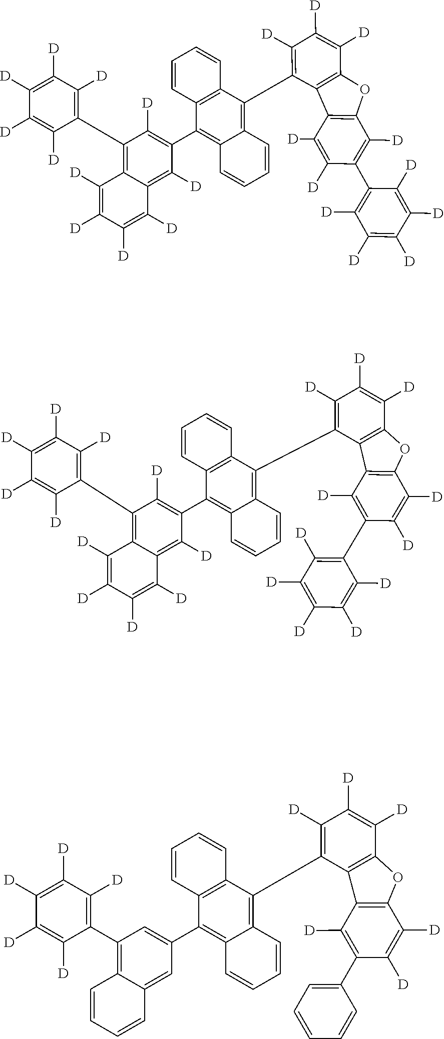

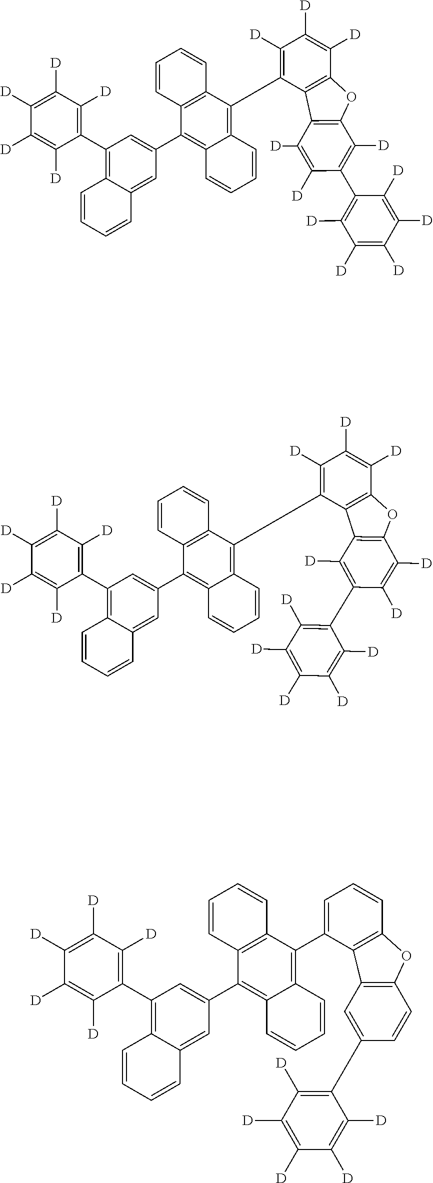 [SMILES: [2H]c1c([2H])c([2H])c(-c2c([2H])c([2H])c3c(oc4c([2H])c([2H])c([2H])c(-c5c6ccccc6c(-c6cc(-c7c([2H])c([2H])c([2H])c([2H])c7[2H])c7ccccc7c6)c6ccccc56)c43)c2[2H])c([2H])c1[2H] ; [2H]c1c([2H])c([2H])c(-c2c([2H])c([2H])c3oc4c([2H])c([2H])c([2H])c(-c5c6ccccc6c(-c6cc(-c7c([2H])c([2H])c([2H])c([2H])c7[2H])c7ccccc7c6)c6ccccc56)c4c3c2[2H])c([2H])c1[2H] ; [2H]c1c([2H])c([2H])c(-c2ccc3oc4cccc(-c5c6ccccc6c(-c6cc(-c7c([2H])c([2H])c([2H])c([2H])c7[2H])c7ccccc7c6)c6ccccc56)c4c3c2)c([2H])c1[2H]]

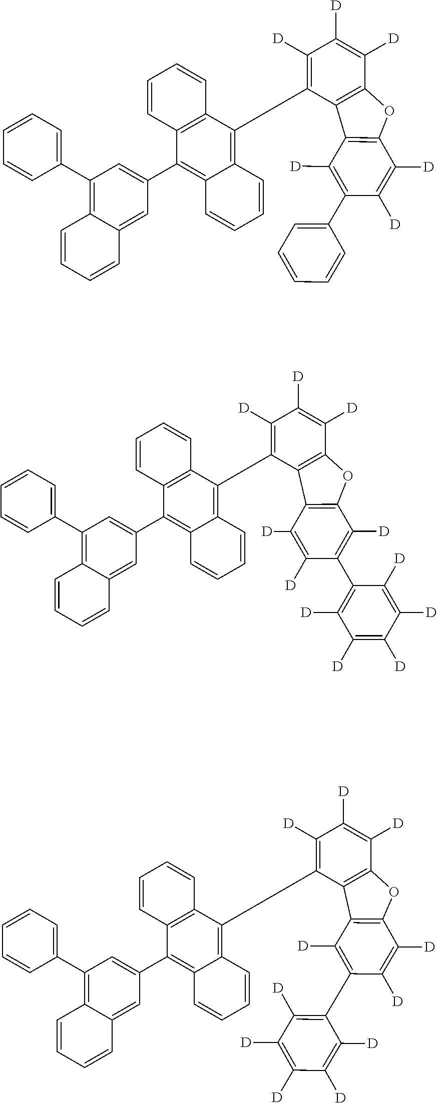 [SMILES: [2H]c1c(-c2ccccc2)c([2H])c2c(oc3c([2H])c([2H])c([2H])c(-c4c5ccccc5c(-c5cc(-c6ccccc6)c6ccccc6c5)c5ccccc45)c32)c1[2H] ; [2H]c1c([2H])c([2H])c(-c2c([2H])c([2H])c3c(oc4c([2H])c([2H])c([2H])c(-c5c6ccccc6c(-c6cc(-c7ccccc7)c7ccccc7c6)c6ccccc56)c43)c2[2H])c([2H])c1[2H] ; [2H]c1c([2H])c([2H])c(-c2c([2H])c([2H])c3oc4c([2H])c([2H])c([2H])c(-c5c6ccccc6c(-c6cc(-c7ccccc7)c7ccccc7c6)c6ccccc56)c4c3c2[2H])c([2H])c1[2H]]

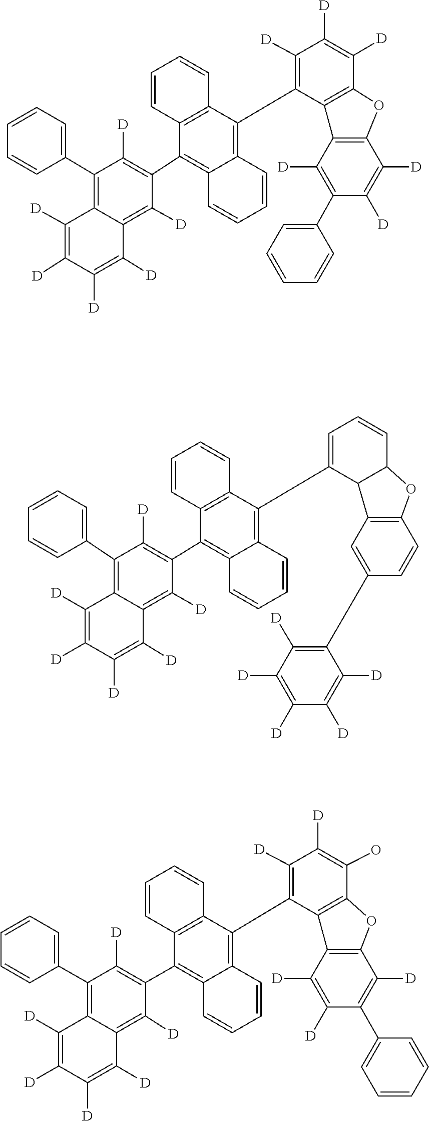

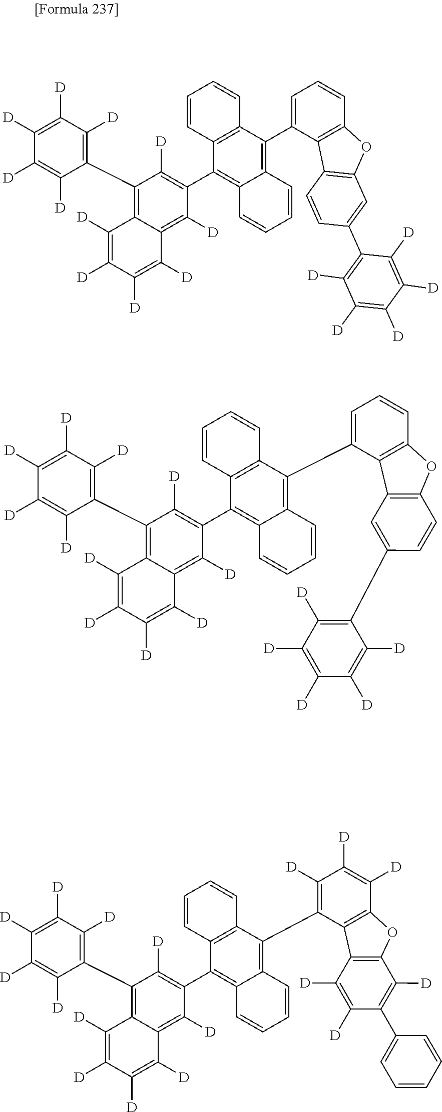

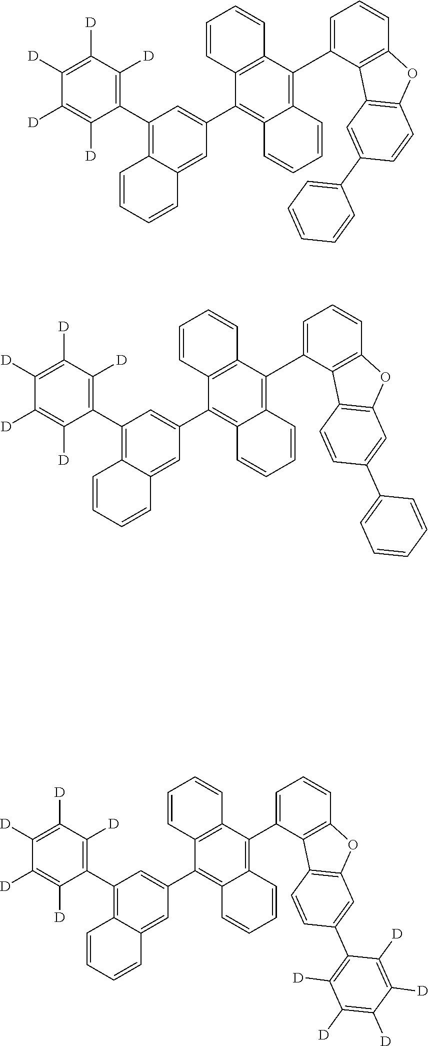 [SMILES: [2H]c1c([2H])c([2H])c(-c2cc(-c3c4ccccc4c(-c4cccc5oc6ccc(-c7ccccc7)cc6c45)c4ccccc34)cc3ccccc23)c([2H])c1[2H] ; [2H]c1c([2H])c([2H])c(-c2cc(-c3c4ccccc4c(-c4cccc5oc6cc(-c7ccccc7)ccc6c45)c4ccccc34)cc3ccccc23)c([2H])c1[2H] ; [2H]c1c([2H])c([2H])c(-c2ccc3c(c2)oc2cccc(-c4c5ccccc5c(-c5cc(-c6c([2H])c([2H])c([2H])c([2H])c6[2H])c6ccccc6c5)c5ccccc45)c23)c([2H])c1[2H]]

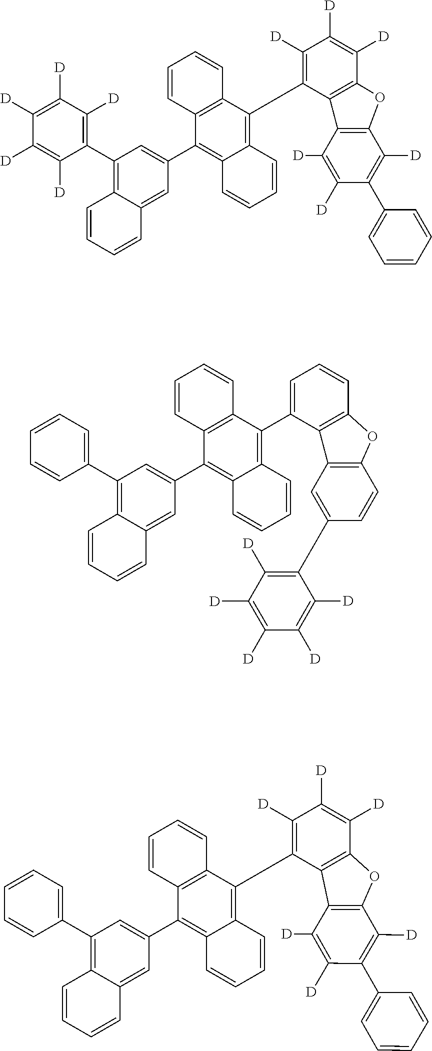 [SMILES: [2H]c1c([2H])c([2H])c(-c2cc(-c3c4ccccc4c(-c4c([2H])c([2H])c([2H])c5oc6c([2H])c(-c7ccccc7)c([2H])c([2H])c6c45)c4ccccc34)cc3ccccc23)c([2H])c1[2H] ; [2H]c1c([2H])c([2H])c(-c2ccc3oc4cccc(-c5c6ccccc6c(-c6cc(-c7ccccc7)c7ccccc7c6)c6ccccc56)c4c3c2)c([2H])c1[2H] ; [2H]c1c([2H])c(-c2c3ccccc3c(-c3cc(-c4ccccc4)c4ccccc4c3)c3ccccc23)c2c(oc3c([2H])c(-c4ccccc4)c([2H])c([2H])c32)c1[2H]]

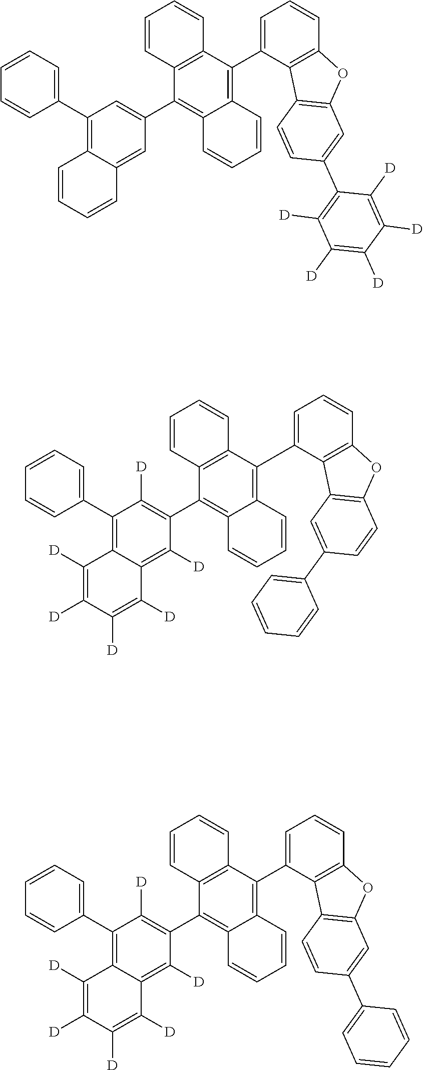 [SMILES: [2H]c1c([2H])c([2H])c(-c2ccc3c(c2)oc2cccc(-c4c5ccccc5c(-c5cc(-c6ccccc6)c6ccccc6c5)c5ccccc45)c23)c([2H])c1[2H] ; [2H]c1c([2H])c([2H])c2c(-c3ccccc3)c([2H])c(-c3c4ccccc4c(-c4cccc5oc6ccc(-c7ccccc7)cc6c45)c4ccccc34)c([2H])c2c1[2H] ; [2H]c1c([2H])c([2H])c2c(-c3ccccc3)c([2H])c(-c3c4ccccc4c(-c4cccc5oc6cc(-c7ccccc7)ccc6c45)c4ccccc34)c([2H])c2c1[2H]]

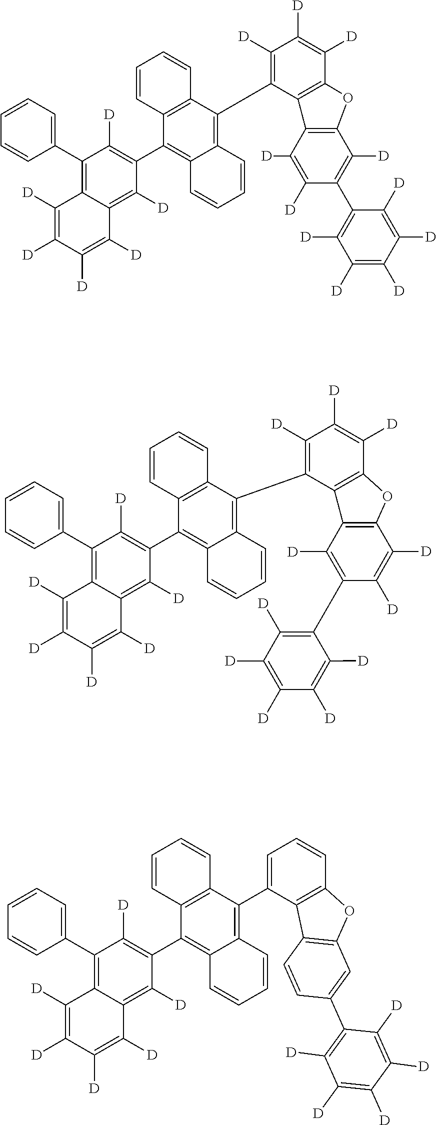

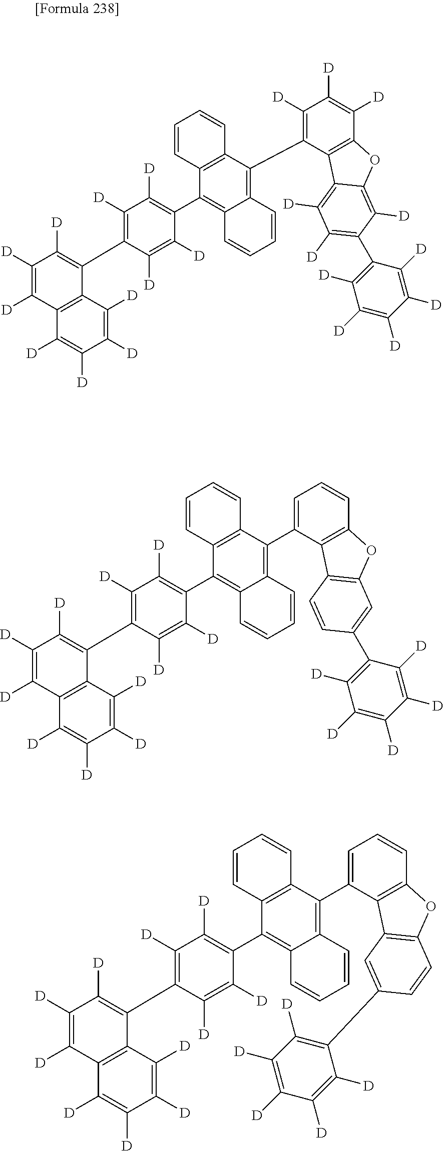

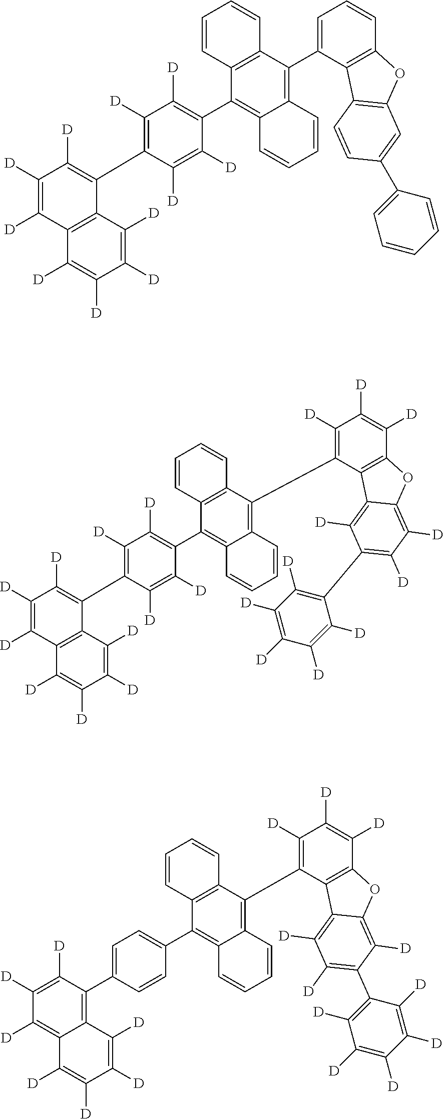

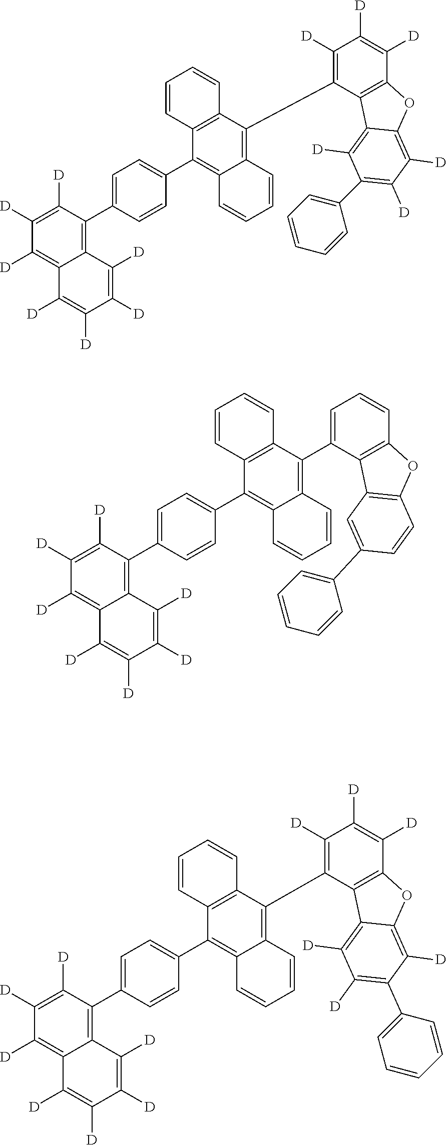 [SMILES: [2H]c1c(-c2ccccc2)c([2H])c2c(oc3c([2H])c([2H])c([2H])c(-c4c5ccccc5c(-c5ccc(-c6c([2H])c([2H])c([2H])c7c([2H])c([2H])c([2H])c([2H])c67)cc5)c5ccccc45)c32)c1[2H] ; [2H]c1c([2H])c([2H])c2c(-c3ccc(-c4c5ccccc5c(-c5cccc6oc7ccc(-c8ccccc8)cc7c56)c5ccccc45)cc3)c([2H])c([2H])c([2H])c2c1[2H] ; [2H]c1c([2H])c(-c2c3ccccc3c(-c3ccc(-c4c([2H])c([2H])c([2H])c5c([2H])c([2H])c([2H])c([2H])c45)cc3)c3ccccc23)c2c(oc3c([2H])c(-c4ccccc4)c([2H])c([2H])c32)c1[2H]]

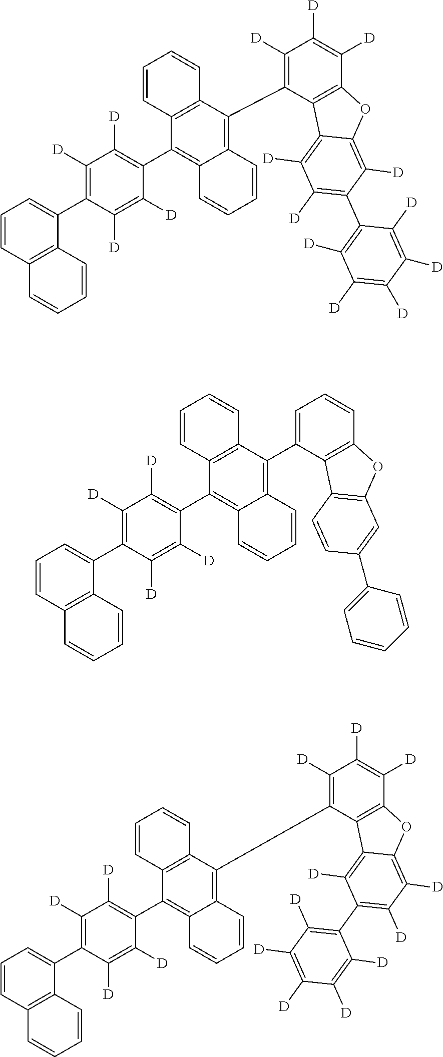 [SMILES: [2H]c1c([2H])c([2H])c(-c2c([2H])c([2H])c3c(oc4c([2H])c([2H])c([2H])c(-c5c6ccccc6c(-c6c([2H])c([2H])c(-c7cccc8ccccc78)c([2H])c6[2H])c6ccccc56)c43)c2[2H])c([2H])c1[2H] ; [2H]c1c([2H])c(-c2c3ccccc3c(-c3cccc4oc5cc(-c6ccccc6)ccc5c34)c3ccccc23)c([2H])c([2H])c1-c1cccc2ccccc12 ; [2H]c1c([2H])c([2H])c(-c2c([2H])c([2H])c3oc4c([2H])c([2H])c([2H])c(-c5c6ccccc6c(-c6c([2H])c([2H])c(-c7cccc8ccccc78)c([2H])c6[2H])c6ccccc56)c4c3c2[2H])c([2H])c1[2H]]

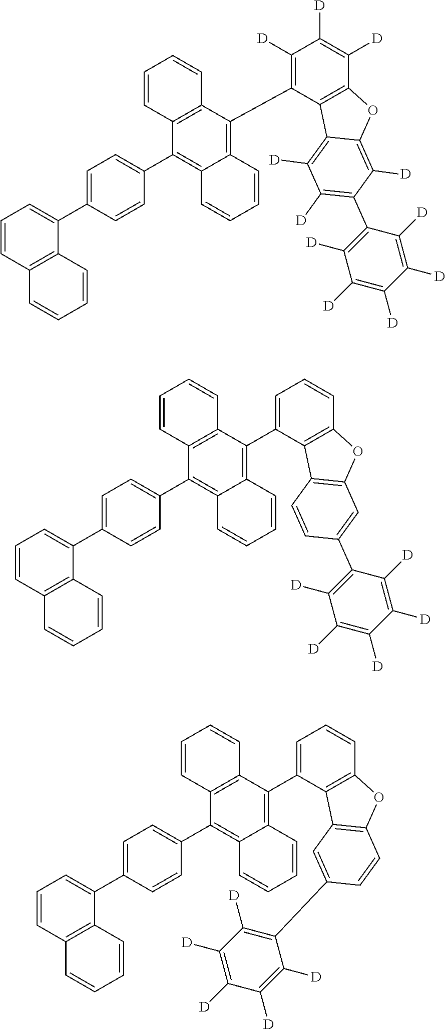 [SMILES: [2H]c1c([2H])c([2H])c(-c2c([2H])c([2H])c3c(oc4c([2H])c([2H])c([2H])c(-c5c6ccccc6c(-c6ccc(-c7cccc8ccccc78)cc6)c6ccccc56)c43)c2[2H])c([2H])c1[2H] ; [2H]c1c([2H])c([2H])c(-c2ccc3c(c2)oc2cccc(-c4c5ccccc5c(-c5ccc(-c6cccc7ccccc67)cc5)c5ccccc45)c23)c([2H])c1[2H] ; [2H]c1c([2H])c([2H])c(-c2ccc3oc4cccc(-c5c6ccccc6c(-c6ccc(-c7cccc8ccccc78)cc6)c6ccccc56)c4c3c2)c([2H])c1[2H]]

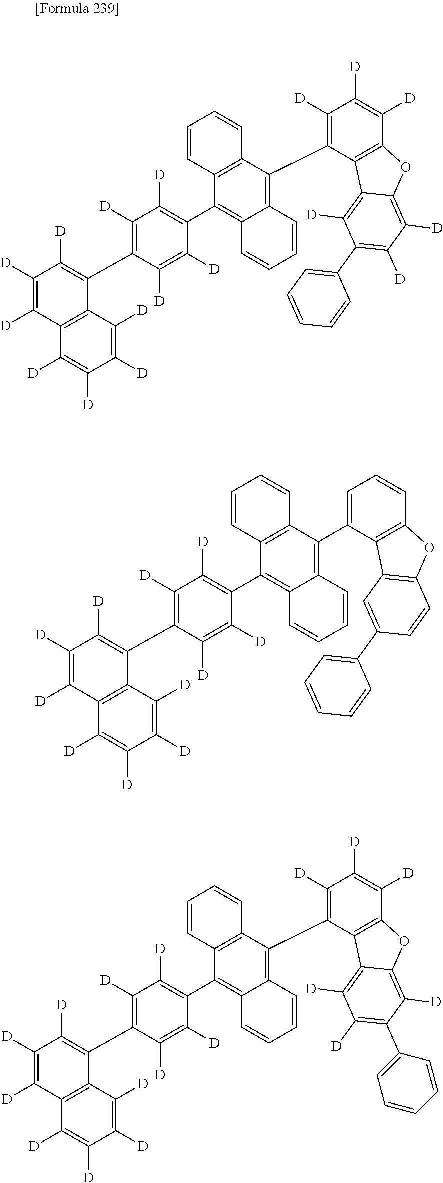

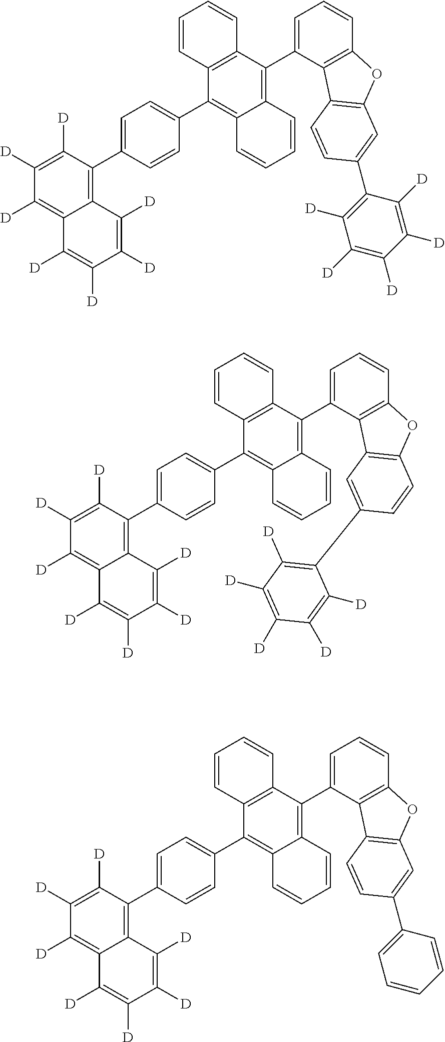 [SMILES: [2H]c1c([2H])c([2H])c(-c2ccc3c(c2)oc2cccc(-c4c5ccccc5c(-c5ccc(-c6c([2H])c([2H])c([2H])c7c([2H])c([2H])c([2H])c([2H])c67)cc5)c5ccccc45)c23)c([2H])c1[2H] ; [2H]c1c([2H])c([2H])c(-c2ccc3oc4cccc(-c5c6ccccc6c(-c6ccc(-c7c([2H])c([2H])c([2H])c8c([2H])c([2H])c([2H])c([2H])c78)cc6)c6ccccc56)c4c3c2)c([2H])c1[2H] ; [2H]c1c([2H])c([2H])c2c(-c3ccc(-c4c5ccccc5c(-c5cccc6oc7cc(-c8ccccc8)ccc7c56)c5ccccc45)cc3)c([2H])c([2H])c([2H])c2c1[2H]]

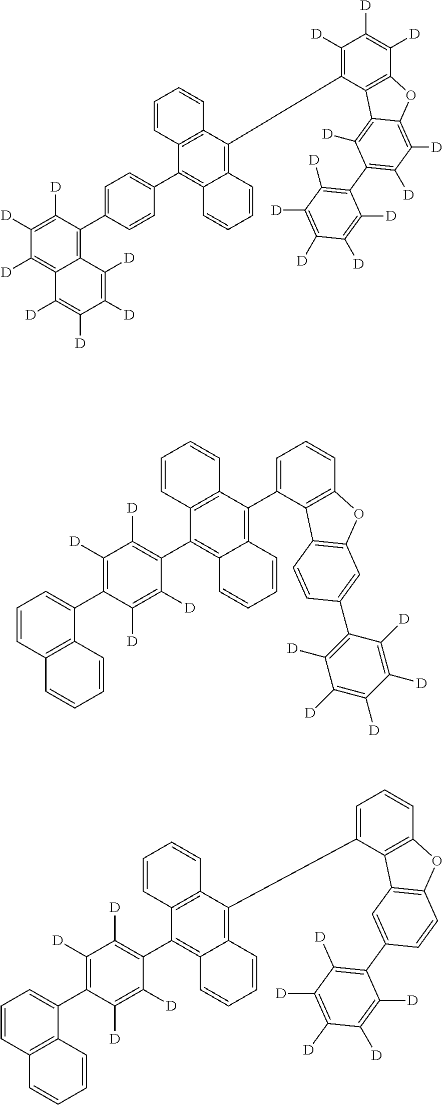 [SMILES: [2H]c1c([2H])c([2H])c(-c2c([2H])c([2H])c3oc4c([2H])c([2H])c([2H])c(-c5c6ccccc6c(-c6ccc(-c7c([2H])c([2H])c([2H])c8c([2H])c([2H])c([2H])c([2H])c78)cc6)c6ccccc56)c4c3c2[2H])c([2H])c1[2H] ; [2H]c1c([2H])c([2H])c(-c2ccc3c(c2)oc2cccc(-c4c5ccccc5c(-c5c([2H])c([2H])c(-c6cccc7ccccc67)c([2H])c5[2H])c5ccccc45)c23)c([2H])c1[2H] ; [2H]c1c([2H])c([2H])c(-c2ccc3oc4cccc(-c5c6ccccc6c(-c6c([2H])c([2H])c(-c7cccc8ccccc78)c([2H])c6[2H])c6ccccc56)c4c3c2)c([2H])c1[2H]]

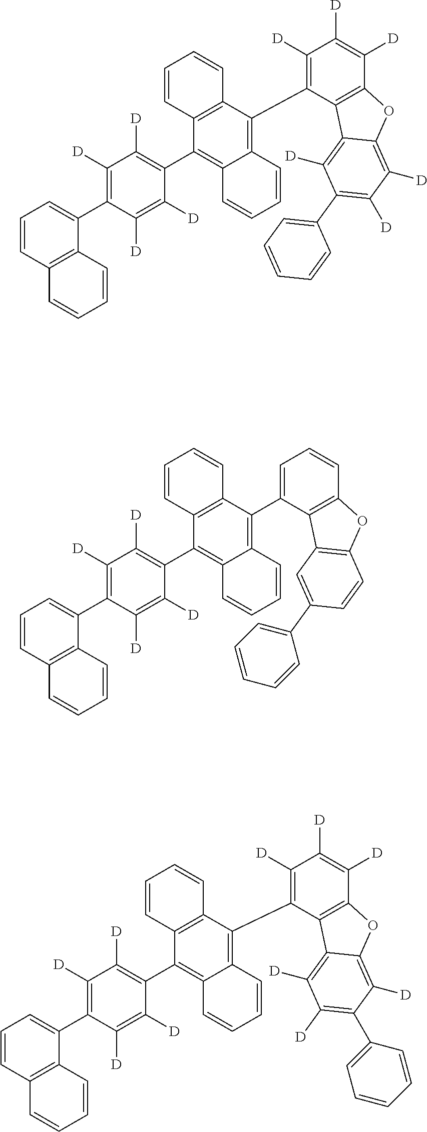 [SMILES: [2H]c1c([2H])c(-c2c3ccccc3c(-c3c([2H])c([2H])c([2H])c4oc5c([2H])c([2H])c(-c6ccccc6)c([2H])c5c34)c3ccccc23)c([2H])c([2H])c1-c1cccc2ccccc12 ; [2H]c1c([2H])c(-c2c3ccccc3c(-c3cccc4oc5ccc(-c6ccccc6)cc5c34)c3ccccc23)c([2H])c([2H])c1-c1cccc2ccccc12 ; [2H]c1c([2H])c(-c2c3ccccc3c(-c3c([2H])c([2H])c([2H])c4oc5c([2H])c(-c6ccccc6)c([2H])c([2H])c5c34)c3ccccc23)c([2H])c([2H])c1-c1cccc2ccccc12]

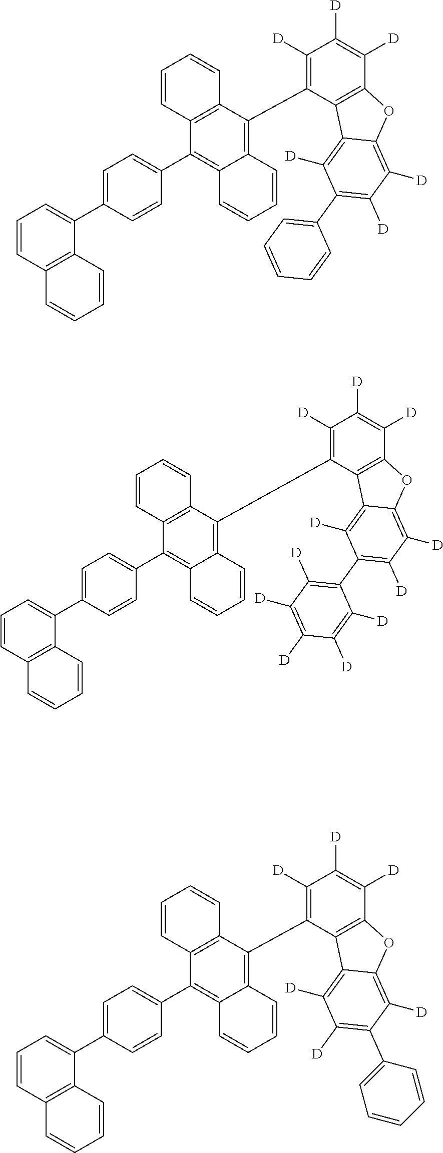 [SMILES: [2H]c1c(-c2ccccc2)c([2H])c2c(oc3c([2H])c([2H])c([2H])c(-c4c5ccccc5c(-c5ccc(-c6cccc7ccccc67)cc5)c5ccccc45)c32)c1[2H] ; [2H]c1c([2H])c([2H])c(-c2c([2H])c([2H])c3oc4c([2H])c([2H])c([2H])c(-c5c6ccccc6c(-c6ccc(-c7cccc8ccccc78)cc6)c6ccccc56)c4c3c2[2H])c([2H])c1[2H] ; [2H]c1c([2H])c(-c2c3ccccc3c(-c3ccc(-c4cccc5ccccc45)cc3)c3ccccc23)c2c(oc3c([2H])c(-c4ccccc4)c([2H])c([2H])c32)c1[2H]]

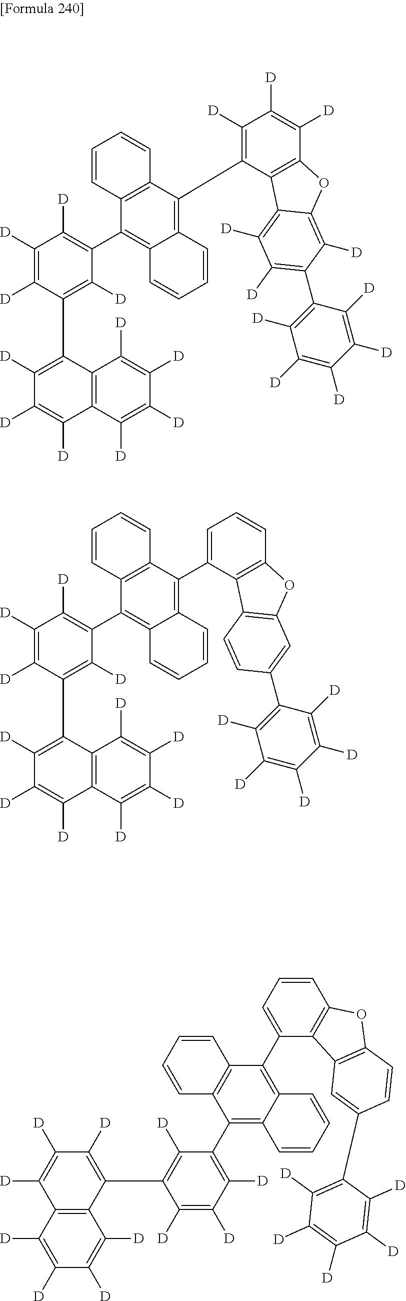

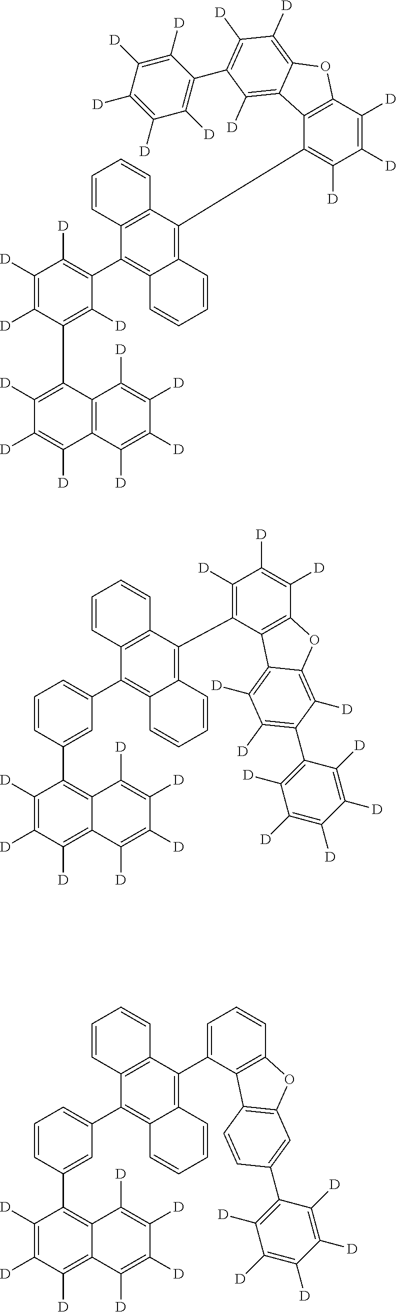

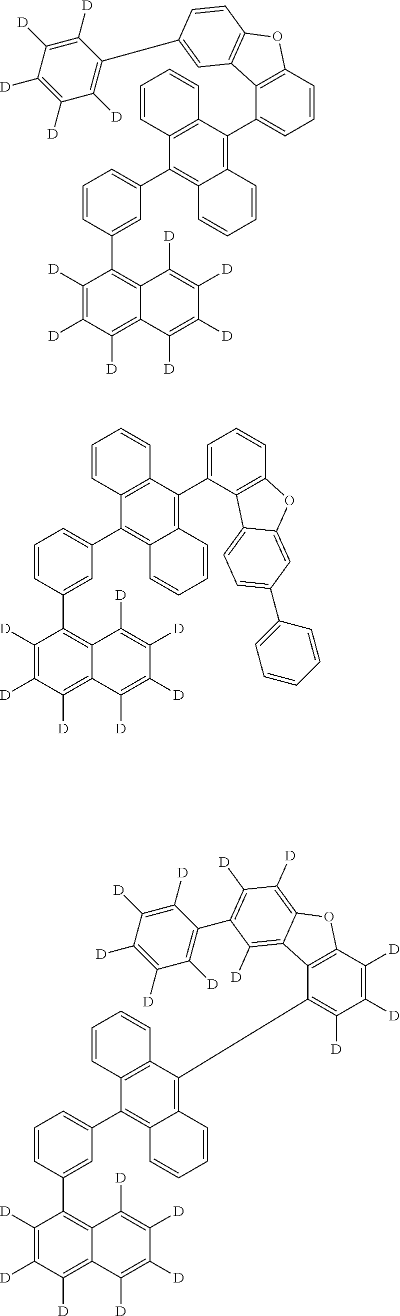 [SMILES: [2H]c1c([2H])c([2H])c(-c2ccc3oc4cccc(-c5c6ccccc6c(-c6cccc(-c7c([2H])c([2H])c([2H])c8c([2H])c([2H])c([2H])c([2H])c78)c6)c6ccccc56)c4c3c2)c([2H])c1[2H] ; [2H]c1c([2H])c([2H])c2c(-c3cccc(-c4c5ccccc5c(-c5cccc6oc7cc(-c8ccccc8)ccc7c56)c5ccccc45)c3)c([2H])c([2H])c([2H])c2c1[2H] ; [2H]c1c([2H])c([2H])c(-c2c([2H])c([2H])c3oc4c([2H])c([2H])c([2H])c(-c5c6ccccc6c(-c6cccc(-c7c([2H])c([2H])c([2H])c8c([2H])c([2H])c([2H])c([2H])c78)c6)c6ccccc56)c4c3c2[2H])c([2H])c1[2H]]

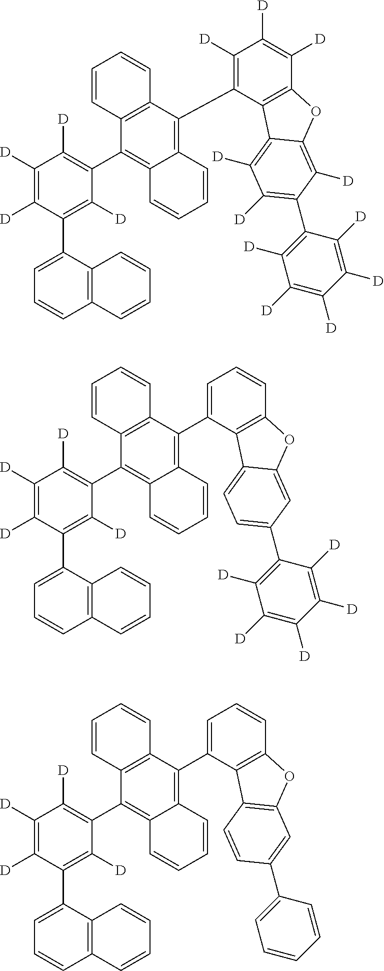 [SMILES: [2H]c1c([2H])c([2H])c(-c2c([2H])c([2H])c3c(oc4c([2H])c([2H])c([2H])c(-c5c6ccccc6c(-c6c([2H])c([2H])c([2H])c(-c7cccc8ccccc78)c6[2H])c6ccccc56)c43)c2[2H])c([2H])c1[2H] ; [2H]c1c([2H])c([2H])c(-c2ccc3c(c2)oc2cccc(-c4c5ccccc5c(-c5c([2H])c([2H])c([2H])c(-c6cccc7ccccc67)c5[2H])c5ccccc45)c23)c([2H])c1[2H] ; [2H]c1c([2H])c(-c2cccc3ccccc23)c([2H])c(-c2c3ccccc3c(-c3cccc4oc5cc(-c6ccccc6)ccc5c34)c3ccccc23)c1[2H]]

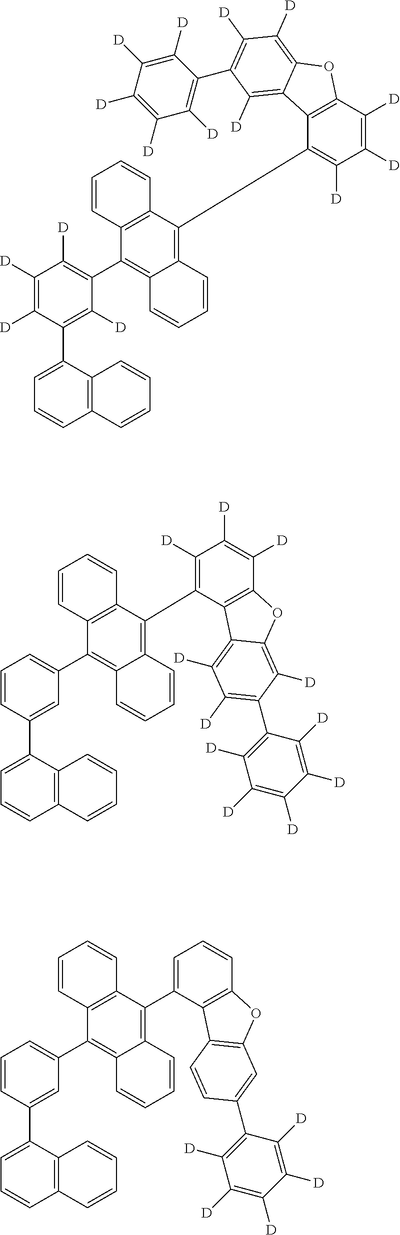 [SMILES: [2H]c1c([2H])c([2H])c(-c2c([2H])c([2H])c3oc4c([2H])c([2H])c([2H])c(-c5c6ccccc6c(-c6c([2H])c([2H])c([2H])c(-c7cccc8ccccc78)c6[2H])c6ccccc56)c4c3c2[2H])c([2H])c1[2H] ; [2H]c1c([2H])c([2H])c(-c2c([2H])c([2H])c3c(oc4c([2H])c([2H])c([2H])c(-c5c6ccccc6c(-c6cccc(-c7cccc8ccccc78)c6)c6ccccc56)c43)c2[2H])c([2H])c1[2H] ; [2H]c1c([2H])c([2H])c(-c2ccc3c(c2)oc2cccc(-c4c5ccccc5c(-c5cccc(-c6cccc7ccccc67)c5)c5ccccc45)c23)c([2H])c1[2H]]

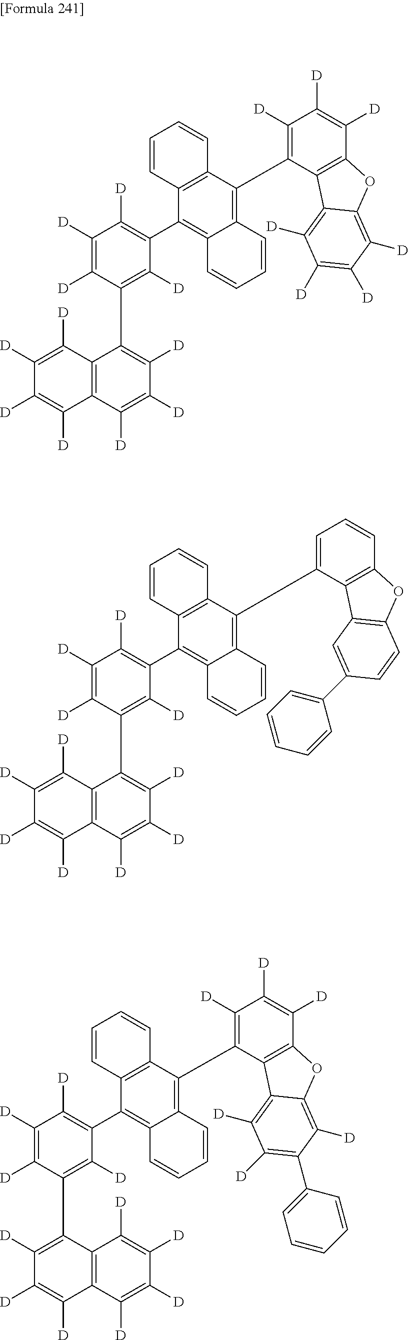

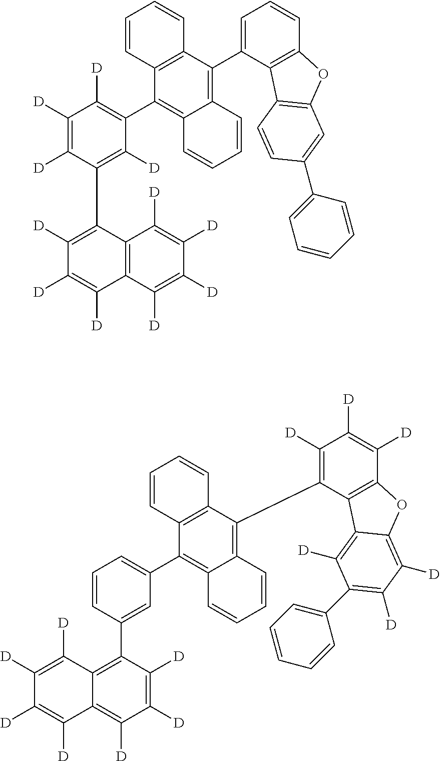 [SMILES: [2H]c1c([2H])c(-c2c3ccccc3c(-c3cccc4oc5cc(-c6ccccc6)ccc5c34)c3ccccc23)c([2H])c(-c2c([2H])c([2H])c([2H])c3c([2H])c([2H])c([2H])c([2H])c23)c1[2H] ; [2H]c1c(-c2ccccc2)c([2H])c2c(oc3c([2H])c([2H])c([2H])c(-c4c5ccccc5c(-c5cccc(-c6c([2H])c([2H])c([2H])c7c([2H])c([2H])c([2H])c([2H])c67)c5)c5ccccc45)c32)c1[2H]]

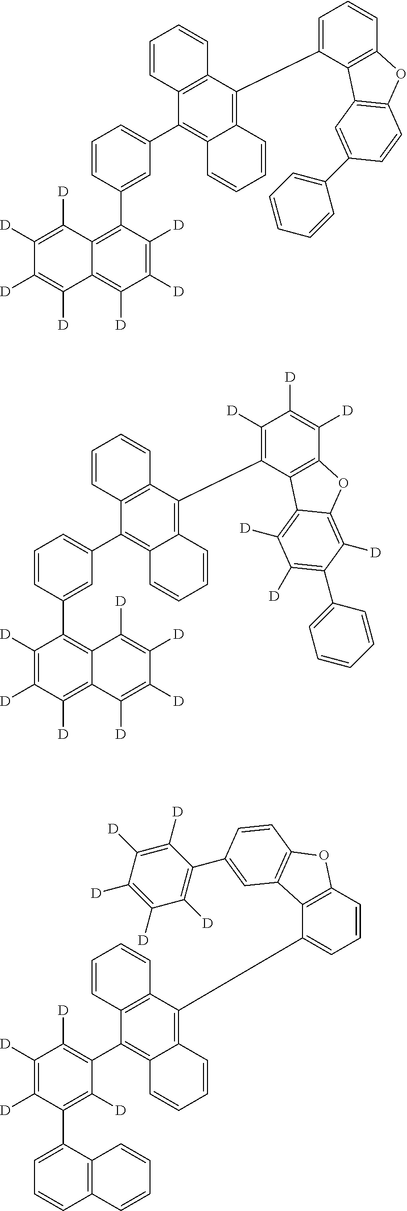 [SMILES: [2H]c1c([2H])c([2H])c2c(-c3cccc(-c4c5ccccc5c(-c5cccc6oc7ccc(-c8ccccc8)cc7c56)c5ccccc45)c3)c([2H])c([2H])c([2H])c2c1[2H] ; [2H]c1c([2H])c(-c2c3ccccc3c(-c3cccc(-c4c([2H])c([2H])c([2H])c5c([2H])c([2H])c([2H])c([2H])c45)c3)c3ccccc23)c2c(oc3c([2H])c(-c4ccccc4)c([2H])c([2H])c32)c1[2H] ; [2H]c1c([2H])c([2H])c(-c2ccc3oc4cccc(-c5c6ccccc6c(-c6c([2H])c([2H])c([2H])c(-c7cccc8ccccc78)c6[2H])c6ccccc56)c4c3c2)c([2H])c1[2H]]

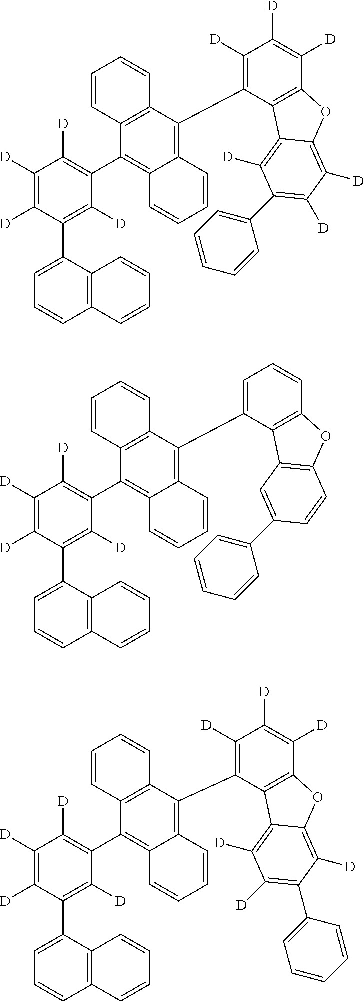 [SMILES: [2H]c1c([2H])c(-c2cccc3ccccc23)c([2H])c(-c2c3ccccc3c(-c3c([2H])c([2H])c([2H])c4oc5c([2H])c([2H])c(-c6ccccc6)c([2H])c5c34)c3ccccc23)c1[2H] ; [2H]c1c([2H])c(-c2cccc3ccccc23)c([2H])c(-c2c3ccccc3c(-c3cccc4oc5ccc(-c6ccccc6)cc5c34)c3ccccc23)c1[2H] ; [2H]c1c([2H])c(-c2cccc3ccccc23)c([2H])c(-c2c3ccccc3c(-c3c([2H])c([2H])c([2H])c4oc5c([2H])c(-c6ccccc6)c([2H])c([2H])c5c34)c3ccccc23)c1[2H]]

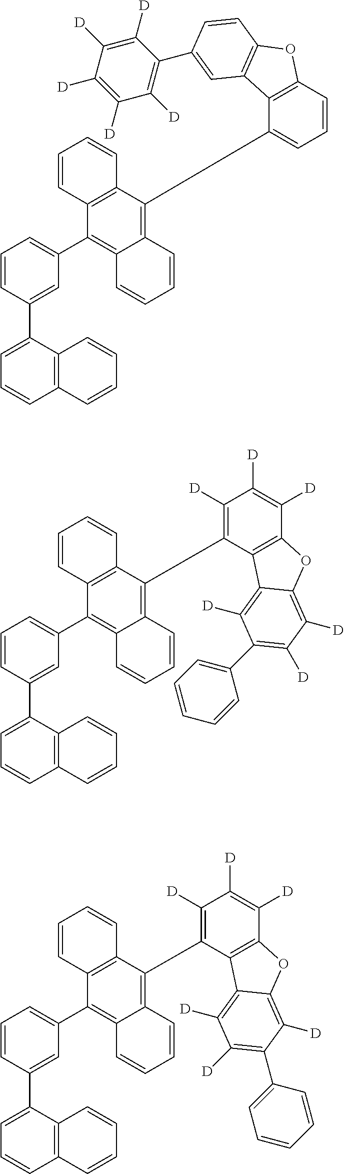 [SMILES: [2H]c1c([2H])c([2H])c(-c2ccc3oc4cccc(-c5c6ccccc6c(-c6cccc(-c7cccc8ccccc78)c6)c6ccccc56)c4c3c2)c([2H])c1[2H] ; [2H]c1c(-c2ccccc2)c([2H])c2c(oc3c([2H])c([2H])c([2H])c(-c4c5ccccc5c(-c5cccc(-c6cccc7ccccc67)c5)c5ccccc45)c32)c1[2H] ; [2H]c1c([2H])c(-c2c3ccccc3c(-c3cccc(-c4cccc5ccccc45)c3)c3ccccc23)c2c(oc3c([2H])c(-c4ccccc4)c([2H])c([2H])c32)c1[2H]]

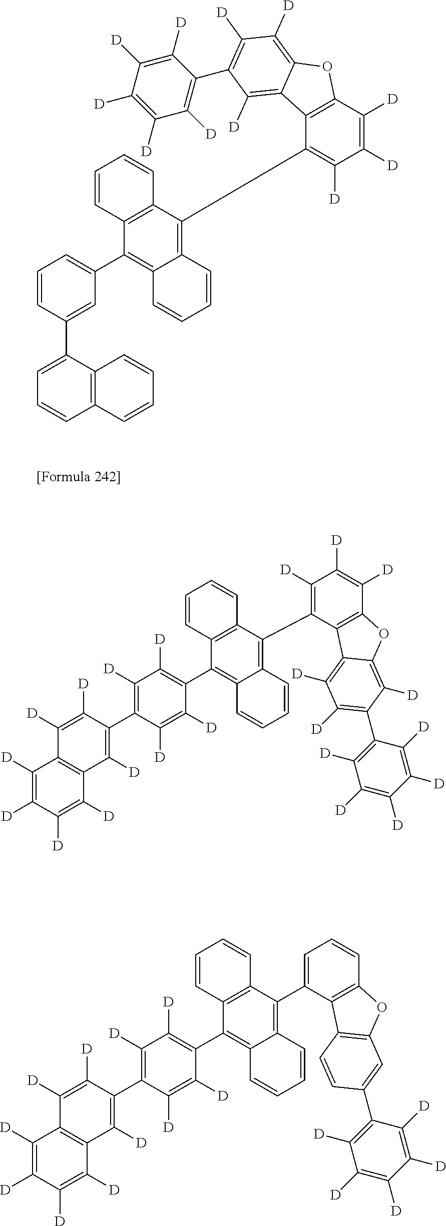

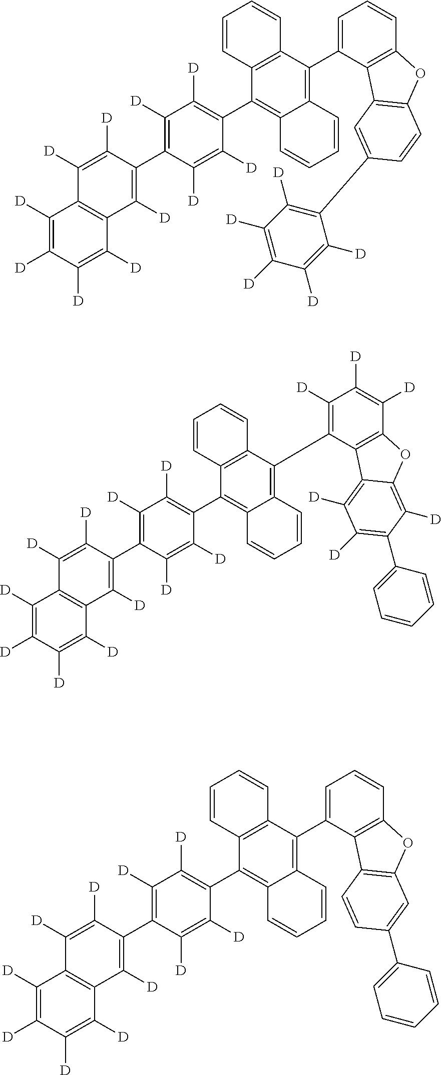 [SMILES: [2H]c1c([2H])c([2H])c(-c2ccc3oc4cccc(-c5c6ccccc6c(-c6c([2H])c([2H])c(-c7c([2H])c([2H])c8c([2H])c([2H])c([2H])c([2H])c8c7[2H])c([2H])c6[2H])c6ccccc56)c4c3c2)c([2H])c1[2H] ; [2H]c1c([2H])c(-c2c3ccccc3c(-c3c([2H])c([2H])c([2H])c4oc5c([2H])c(-c6ccccc6)c([2H])c([2H])c5c34)c3ccccc23)c([2H])c([2H])c1-c1c([2H])c([2H])c2c([2H])c([2H])c([2H])c([2H])c2c1[2H] ; [2H]c1c([2H])c(-c2c3ccccc3c(-c3cccc4oc5cc(-c6ccccc6)ccc5c34)c3ccccc23)c([2H])c([2H])c1-c1c([2H])c([2H])c2c([2H])c([2H])c([2H])c([2H])c2c1[2H]]

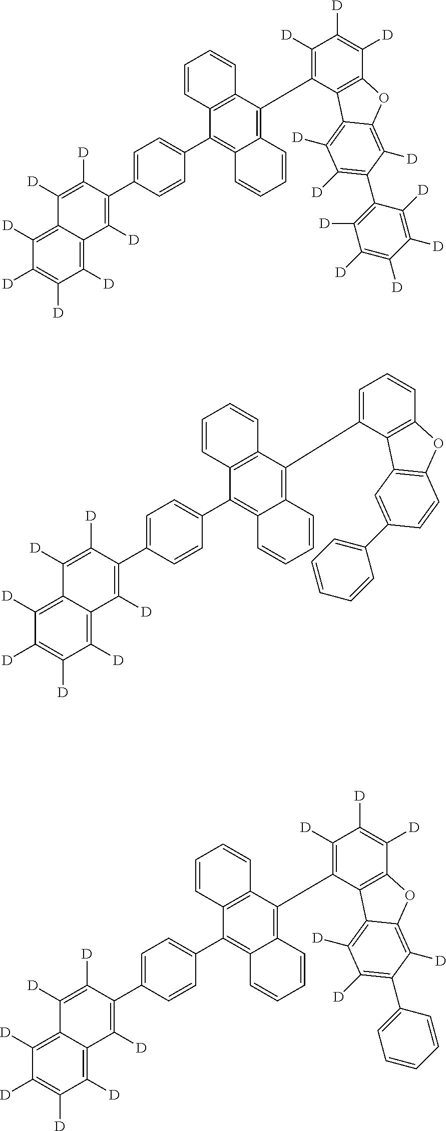 [SMILES: [2H]c1c([2H])c([2H])c(-c2c([2H])c([2H])c3c(oc4c([2H])c([2H])c([2H])c(-c5c6ccccc6c(-c6ccc(-c7c([2H])c([2H])c8c([2H])c([2H])c([2H])c([2H])c8c7[2H])cc6)c6ccccc56)c43)c2[2H])c([2H])c1[2H] ; [2H]c1c([2H])c([2H])c2c([2H])c(-c3ccc(-c4c5ccccc5c(-c5cccc6oc7ccc(-c8ccccc8)cc7c56)c5ccccc45)cc3)c([2H])c([2H])c2c1[2H] ; [2H]c1c([2H])c(-c2c3ccccc3c(-c3ccc(-c4c([2H])c([2H])c5c([2H])c([2H])c([2H])c([2H])c5c4[2H])cc3)c3ccccc23)c2c(oc3c([2H])c(-c4ccccc4)c([2H])c([2H])c32)c1[2H]]

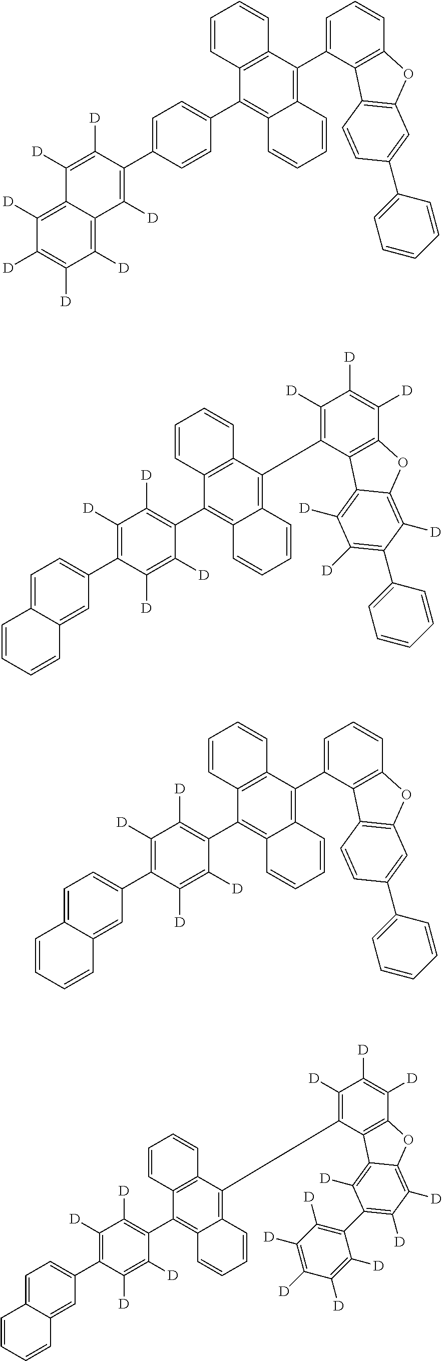

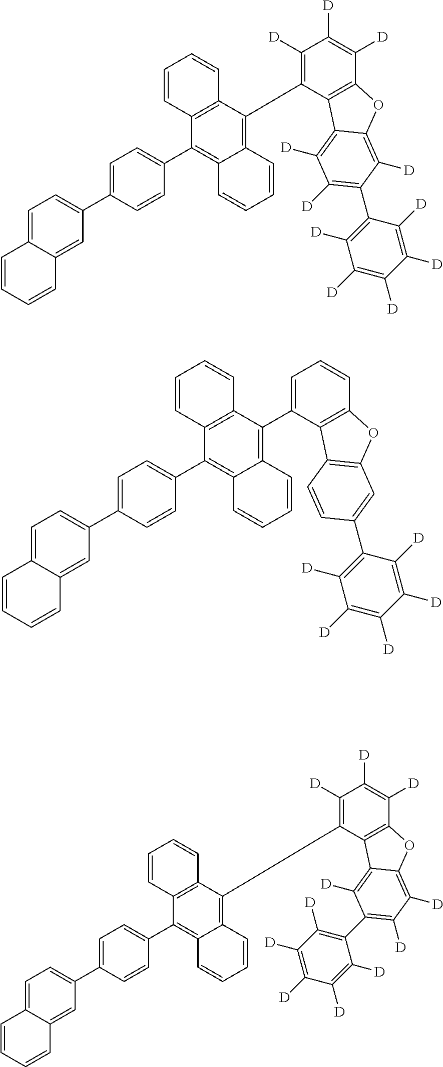 [SMILES: [2H]c1c([2H])c([2H])c(-c2c([2H])c([2H])c3c(oc4c([2H])c([2H])c([2H])c(-c5c6ccccc6c(-c6ccc(-c7ccc8ccccc8c7)cc6)c6ccccc56)c43)c2[2H])c([2H])c1[2H] ; [2H]c1c([2H])c([2H])c(-c2ccc3c(c2)oc2cccc(-c4c5ccccc5c(-c5ccc(-c6ccc7ccccc7c6)cc5)c5ccccc45)c23)c([2H])c1[2H] ; [2H]c1c([2H])c([2H])c(-c2c([2H])c([2H])c3oc4c([2H])c([2H])c([2H])c(-c5c6ccccc6c(-c6ccc(-c7ccc8ccccc8c7)cc6)c6ccccc56)c4c3c2[2H])c([2H])c1[2H]]

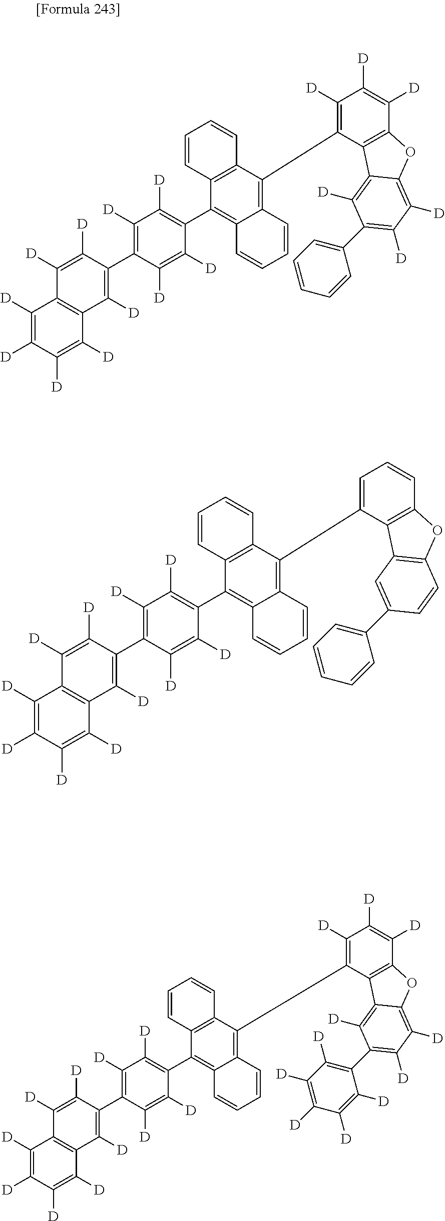

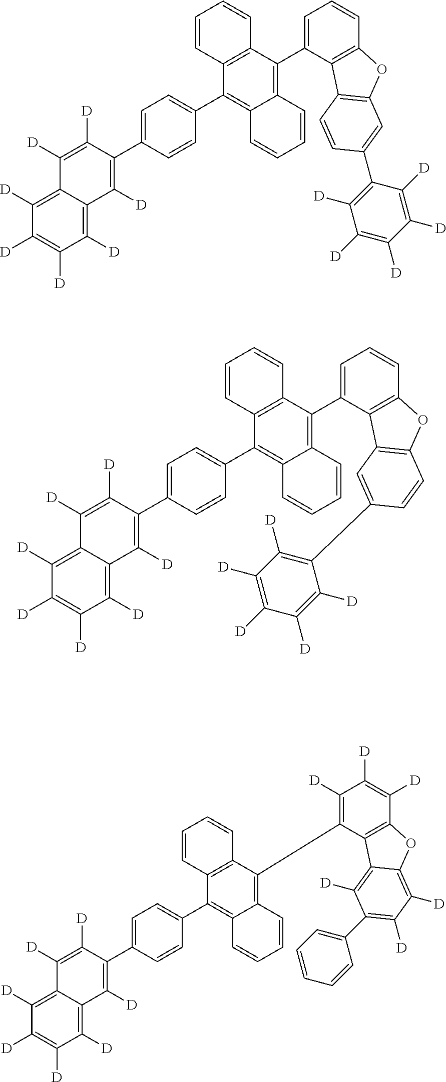 [SMILES: [2H]c1c([2H])c([2H])c(-c2ccc3c(c2)oc2cccc(-c4c5ccccc5c(-c5ccc(-c6c([2H])c([2H])c7c([2H])c([2H])c([2H])c([2H])c7c6[2H])cc5)c5ccccc45)c23)c([2H])c1[2H] ; [2H]c1c([2H])c([2H])c(-c2ccc3oc4cccc(-c5c6ccccc6c(-c6ccc(-c7c([2H])c([2H])c8c([2H])c([2H])c([2H])c([2H])c8c7[2H])cc6)c6ccccc56)c4c3c2)c([2H])c1[2H] ; [2H]c1c(-c2ccccc2)c([2H])c2c(oc3c([2H])c([2H])c([2H])c(-c4c5ccccc5c(-c5ccc(-c6c([2H])c([2H])c7c([2H])c([2H])c([2H])c([2H])c7c6[2H])cc5)c5ccccc45)c32)c1[2H]]

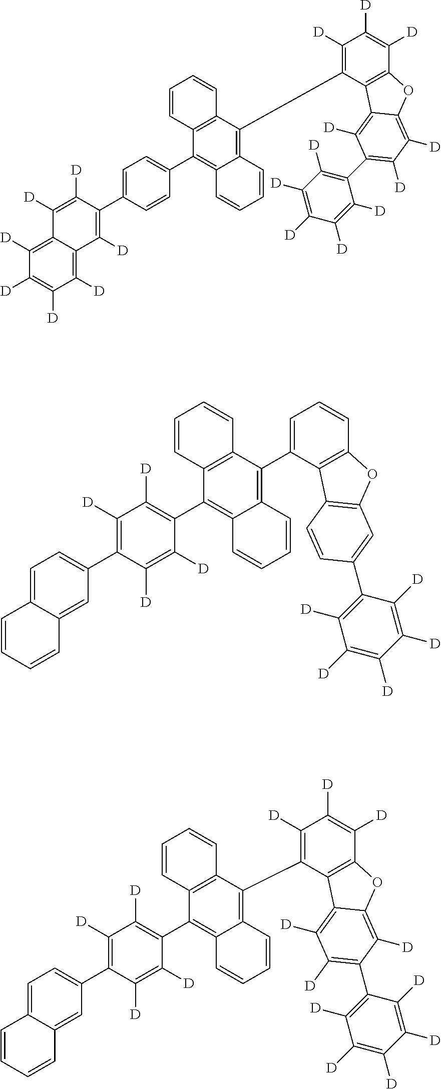 [SMILES: [2H]c1c([2H])c([2H])c(-c2c([2H])c([2H])c3oc4c([2H])c([2H])c([2H])c(-c5c6ccccc6c(-c6ccc(-c7c([2H])c([2H])c8c([2H])c([2H])c([2H])c([2H])c8c7[2H])cc6)c6ccccc56)c4c3c2[2H])c([2H])c1[2H] ; [2H]c1c([2H])c([2H])c(-c2ccc3c(c2)oc2cccc(-c4c5ccccc5c(-c5c([2H])c([2H])c(-c6ccc7ccccc7c6)c([2H])c5[2H])c5ccccc45)c23)c([2H])c1[2H] ; [2H]c1c([2H])c([2H])c(-c2c([2H])c([2H])c3c(oc4c([2H])c([2H])c([2H])c(-c5c6ccccc6c(-c6c([2H])c([2H])c(-c7ccc8ccccc8c7)c([2H])c6[2H])c6ccccc56)c43)c2[2H])c([2H])c1[2H]]

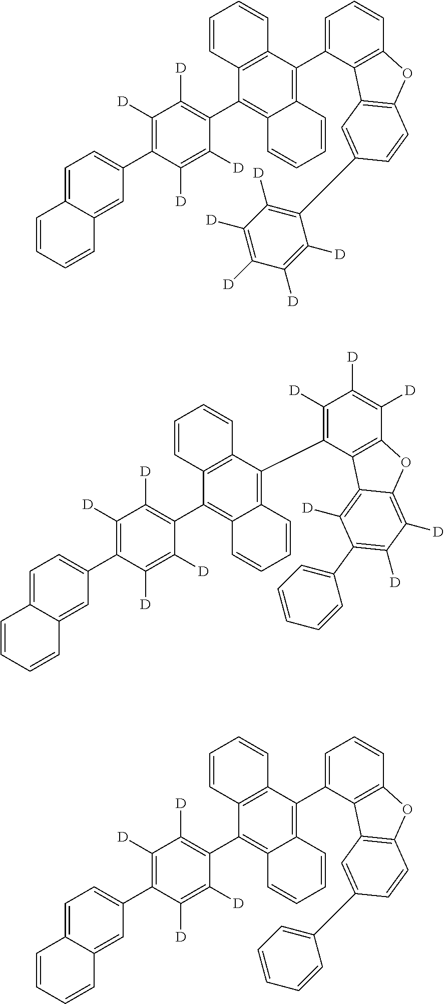 [SMILES: [2H]c1c([2H])c([2H])c(-c2ccc3oc4cccc(-c5c6ccccc6c(-c6c([2H])c([2H])c(-c7ccc8ccccc8c7)c([2H])c6[2H])c6ccccc56)c4c3c2)c([2H])c1[2H] ; [2H]c1c([2H])c(-c2c3ccccc3c(-c3c([2H])c([2H])c([2H])c4oc5c([2H])c([2H])c(-c6ccccc6)c([2H])c5c34)c3ccccc23)c([2H])c([2H])c1-c1ccc2ccccc2c1 ; [2H]c1c([2H])c(-c2c3ccccc3c(-c3cccc4oc5ccc(-c6ccccc6)cc5c34)c3ccccc23)c([2H])c([2H])c1-c1ccc2ccccc2c1]

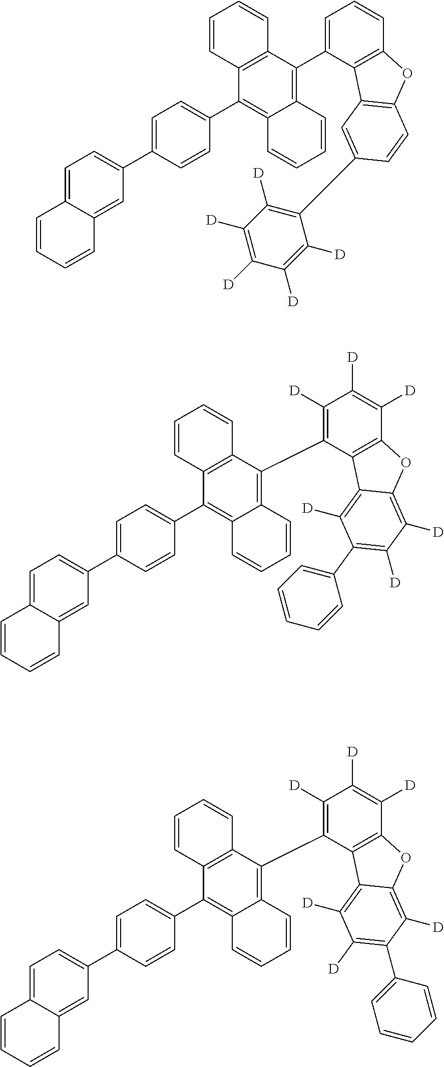 [SMILES: [2H]c1c([2H])c([2H])c(-c2ccc3oc4cccc(-c5c6ccccc6c(-c6ccc(-c7ccc8ccccc8c7)cc6)c6ccccc56)c4c3c2)c([2H])c1[2H] ; [2H]c1c(-c2ccccc2)c([2H])c2c(oc3c([2H])c([2H])c([2H])c(-c4c5ccccc5c(-c5ccc(-c6ccc7ccccc7c6)cc5)c5ccccc45)c32)c1[2H] ; [2H]c1c([2H])c(-c2c3ccccc3c(-c3ccc(-c4ccc5ccccc5c4)cc3)c3ccccc23)c2c(oc3c([2H])c(-c4ccccc4)c([2H])c([2H])c32)c1[2H]]

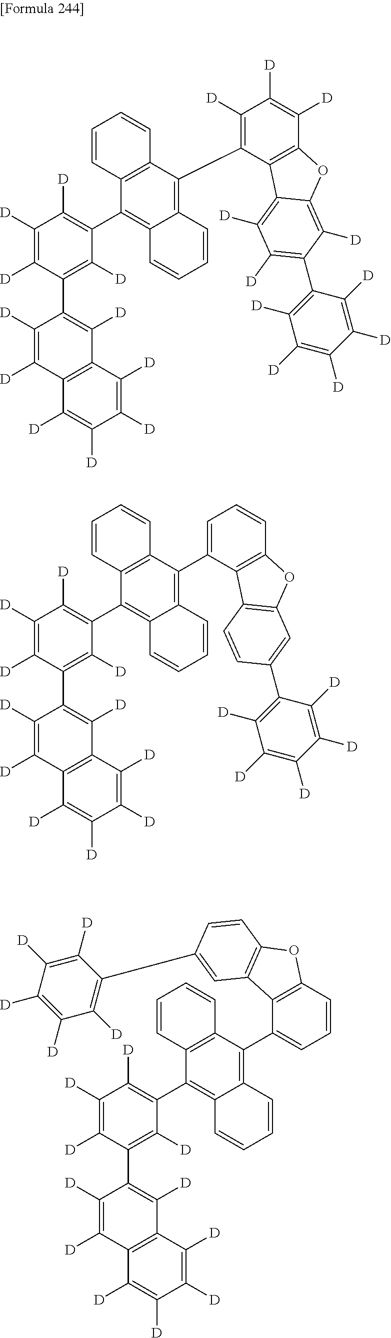

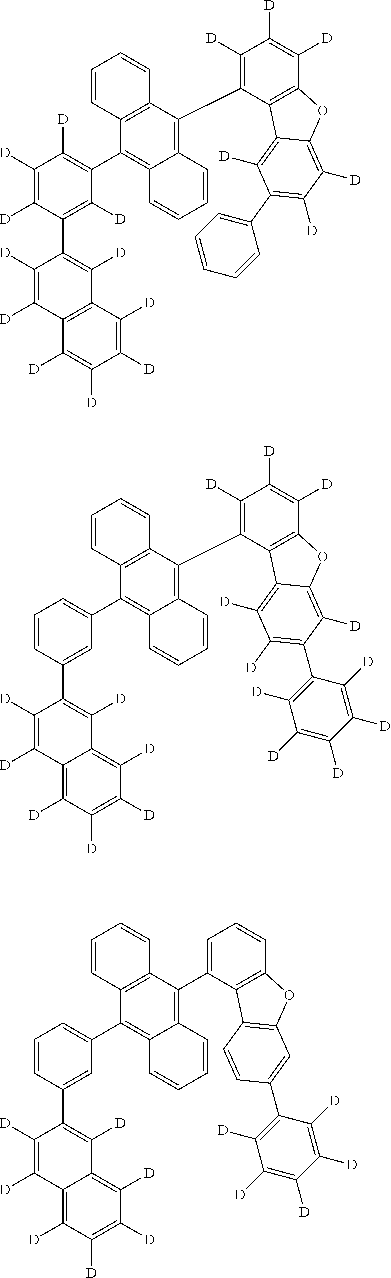

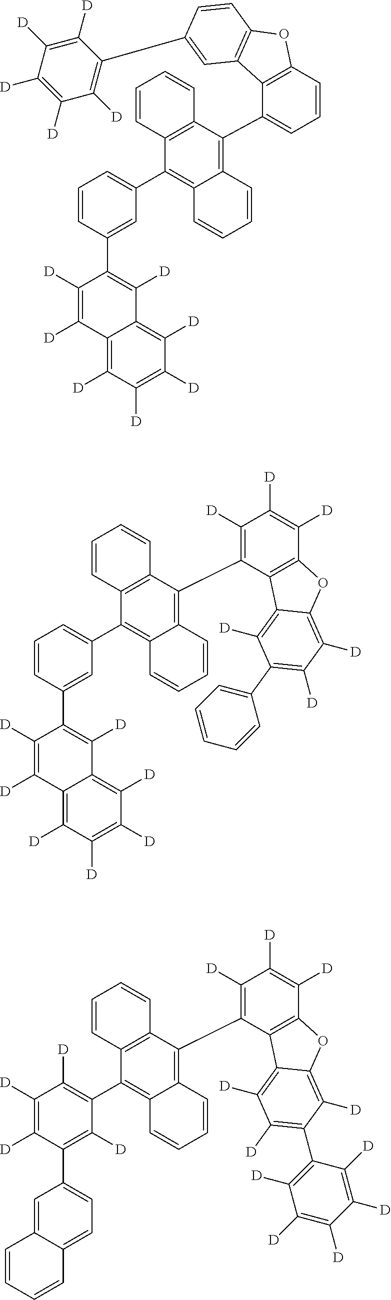 [SMILES: [2H]c1c([2H])c([2H])c(-c2ccc3oc4cccc(-c5c6ccccc6c(-c6cccc(-c7c([2H])c([2H])c8c([2H])c([2H])c([2H])c([2H])c8c7[2H])c6)c6ccccc56)c4c3c2)c([2H])c1[2H] ; [2H]c1c(-c2ccccc2)c([2H])c2c(oc3c([2H])c([2H])c([2H])c(-c4c5ccccc5c(-c5cccc(-c6c([2H])c([2H])c7c([2H])c([2H])c([2H])c([2H])c7c6[2H])c5)c5ccccc45)c32)c1[2H] ; [2H]c1c([2H])c([2H])c(-c2c([2H])c([2H])c3c(oc4c([2H])c([2H])c([2H])c(-c5c6ccccc6c(-c6c([2H])c([2H])c([2H])c(-c7ccc8ccccc8c7)c6[2H])c6ccccc56)c43)c2[2H])c([2H])c1[2H]]

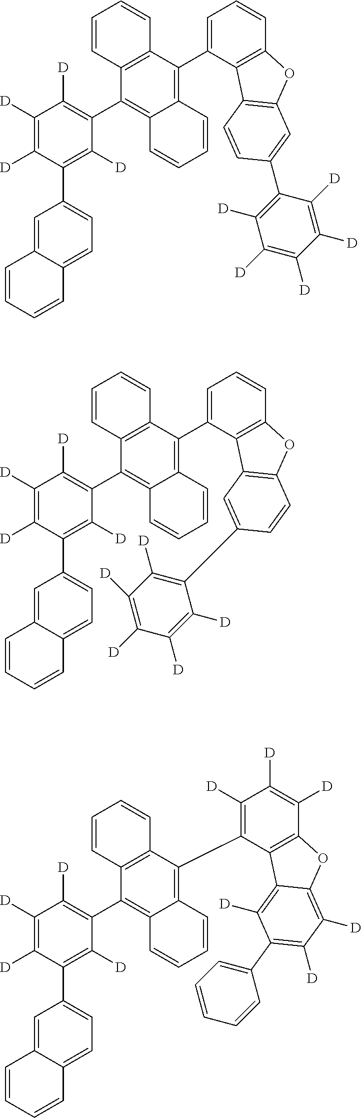 [SMILES: [2H]c1c([2H])c([2H])c(-c2ccc3c(c2)oc2cccc(-c4c5ccccc5c(-c5c([2H])c([2H])c([2H])c(-c6ccc7ccccc7c6)c5[2H])c5ccccc45)c23)c([2H])c1[2H] ; [2H]c1c([2H])c([2H])c(-c2ccc3oc4cccc(-c5c6ccccc6c(-c6c([2H])c([2H])c([2H])c(-c7ccc8ccccc8c7)c6[2H])c6ccccc56)c4c3c2)c([2H])c1[2H] ; [2H]c1c([2H])c(-c2ccc3ccccc3c2)c([2H])c(-c2c3ccccc3c(-c3c([2H])c([2H])c([2H])c4oc5c([2H])c([2H])c(-c6ccccc6)c([2H])c5c34)c3ccccc23)c1[2H]]

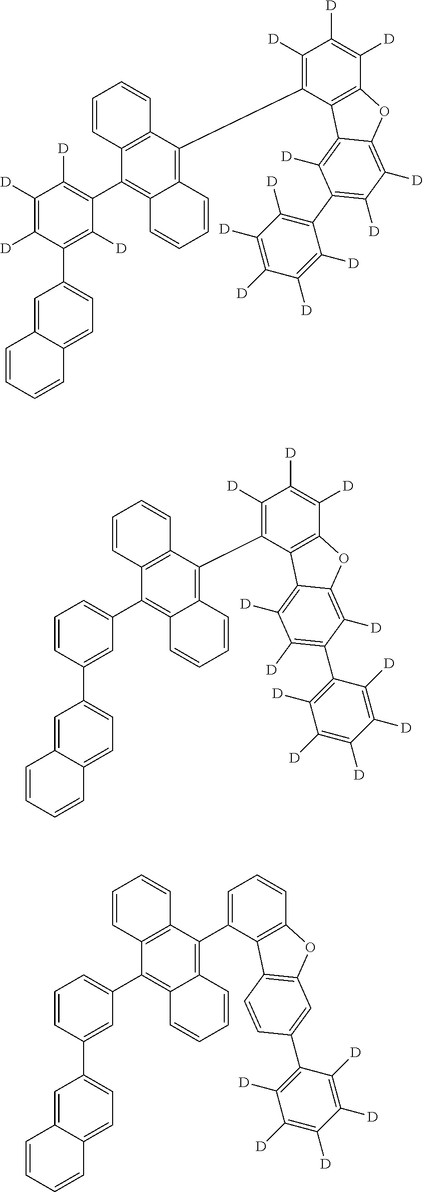 [SMILES: [2H]c1c([2H])c([2H])c(-c2c([2H])c([2H])c3oc4c([2H])c([2H])c([2H])c(-c5c6ccccc6c(-c6c([2H])c([2H])c([2H])c(-c7ccc8ccccc8c7)c6[2H])c6ccccc56)c4c3c2[2H])c([2H])c1[2H] ; [2H]c1c([2H])c([2H])c(-c2c([2H])c([2H])c3c(oc4c([2H])c([2H])c([2H])c(-c5c6ccccc6c(-c6cccc(-c7ccc8ccccc8c7)c6)c6ccccc56)c43)c2[2H])c([2H])c1[2H] ; [2H]c1c([2H])c([2H])c(-c2ccc3c(c2)oc2cccc(-c4c5ccccc5c(-c5cccc(-c6ccc7ccccc7c6)c5)c5ccccc45)c23)c([2H])c1[2H]]

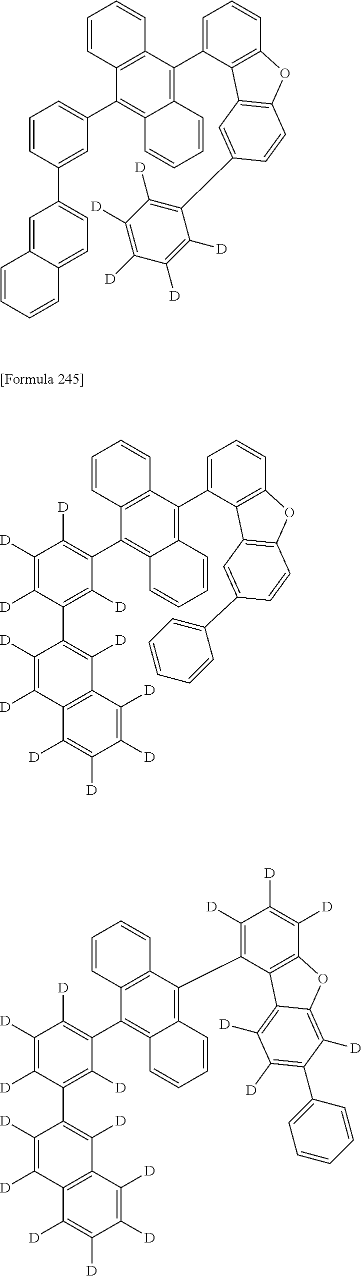 [SMILES: [2H]c1c([2H])c([2H])c(-c2ccc3oc4cccc(-c5c6ccccc6c(-c6cccc(-c7ccc8ccccc8c7)c6)c6ccccc56)c4c3c2)c([2H])c1[2H] ; [2H]c1c([2H])c(-c2c([2H])c([2H])c3c([2H])c([2H])c([2H])c([2H])c3c2[2H])c([2H])c(-c2c3ccccc3c(-c3cccc4oc5ccc(-c6ccccc6)cc5c34)c3ccccc23)c1[2H] ; [2H]c1c([2H])c(-c2c([2H])c([2H])c3c([2H])c([2H])c([2H])c([2H])c3c2[2H])c([2H])c(-c2c3ccccc3c(-c3c([2H])c([2H])c([2H])c4oc5c([2H])c(-c6ccccc6)c([2H])c([2H])c5c34)c3ccccc23)c1[2H]]

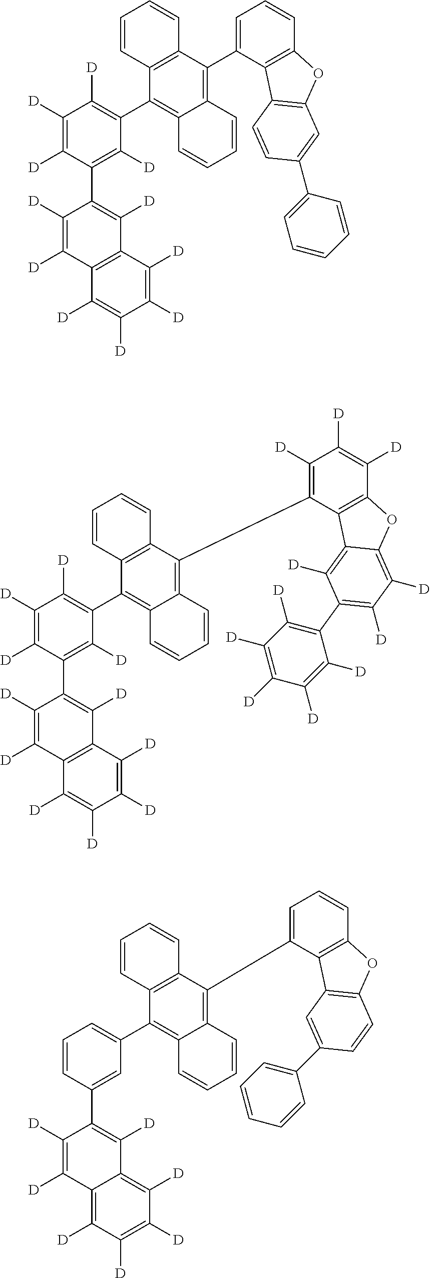 [SMILES: [2H]c1c([2H])c(-c2c([2H])c([2H])c3c([2H])c([2H])c([2H])c([2H])c3c2[2H])c([2H])c(-c2c3ccccc3c(-c3cccc4oc5cc(-c6ccccc6)ccc5c34)c3ccccc23)c1[2H] ; [2H]c1c([2H])c([2H])c(-c2c([2H])c([2H])c3oc4c([2H])c([2H])c([2H])c(-c5c6ccccc6c(-c6c([2H])c([2H])c([2H])c(-c7c([2H])c([2H])c8c([2H])c([2H])c([2H])c([2H])c8c7[2H])c6[2H])c6ccccc56)c4c3c2[2H])c([2H])c1[2H] ; [2H]c1c([2H])c([2H])c2c([2H])c(-c3cccc(-c4c5ccccc5c(-c5cccc6oc7ccc(-c8ccccc8)cc7c56)c5ccccc45)c3)c([2H])c([2H])c2c1[2H]]

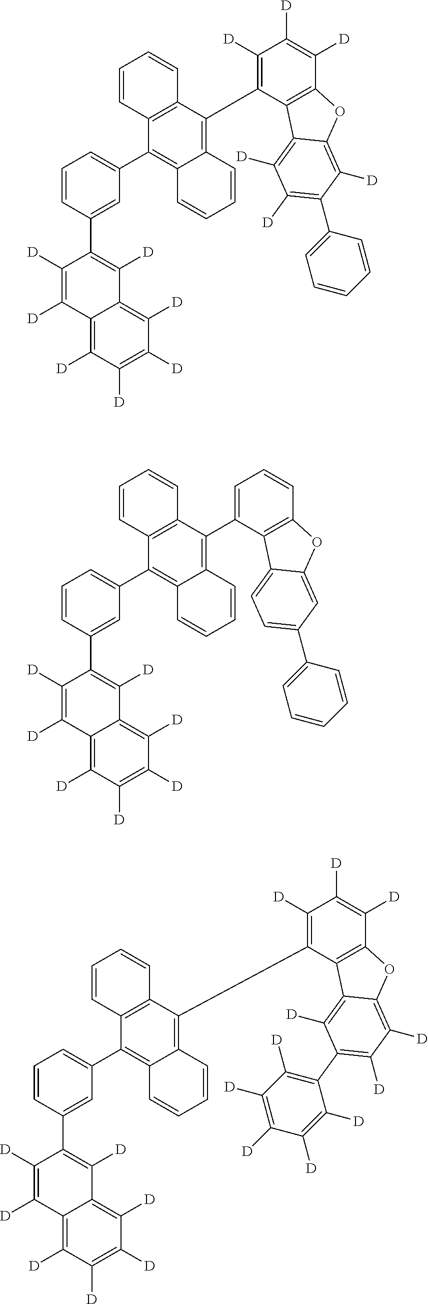 [SMILES: [2H]c1c([2H])c(-c2c3ccccc3c(-c3cccc(-c4c([2H])c([2H])c5c([2H])c([2H])c([2H])c([2H])c5c4[2H])c3)c3ccccc23)c2c(oc3c([2H])c(-c4ccccc4)c([2H])c([2H])c32)c1[2H] ; [2H]c1c([2H])c([2H])c2c([2H])c(-c3cccc(-c4c5ccccc5c(-c5cccc6oc7cc(-c8ccccc8)ccc7c56)c5ccccc45)c3)c([2H])c([2H])c2c1[2H] ; [2H]c1c([2H])c([2H])c(-c2c([2H])c([2H])c3oc4c([2H])c([2H])c([2H])c(-c5c6ccccc6c(-c6cccc(-c7c([2H])c([2H])c8c([2H])c([2H])c([2H])c([2H])c8c7[2H])c6)c6ccccc56)c4c3c2[2H])c([2H])c1[2H]]

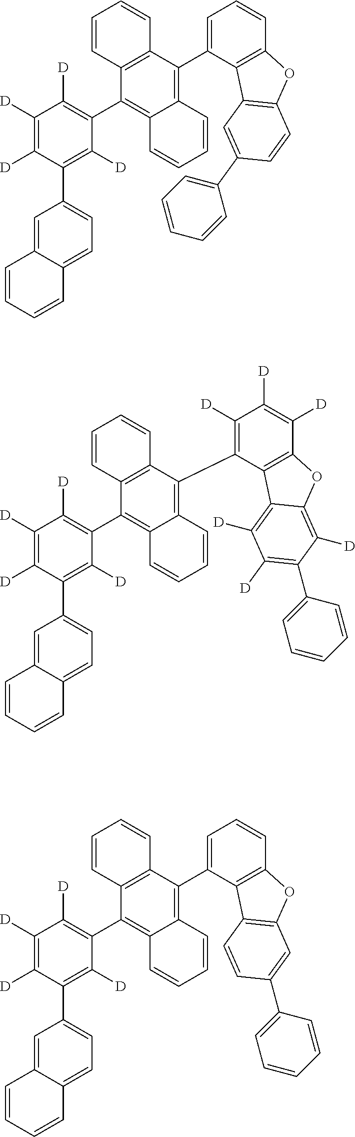 [SMILES: [2H]c1c([2H])c(-c2ccc3ccccc3c2)c([2H])c(-c2c3ccccc3c(-c3cccc4oc5ccc(-c6ccccc6)cc5c34)c3ccccc23)c1[2H] ; [2H]c1c([2H])c(-c2ccc3ccccc3c2)c([2H])c(-c2c3ccccc3c(-c3c([2H])c([2H])c([2H])c4oc5c([2H])c(-c6ccccc6)c([2H])c([2H])c5c34)c3ccccc23)c1[2H] ; [2H]c1c([2H])c(-c2ccc3ccccc3c2)c([2H])c(-c2c3ccccc3c(-c3cccc4oc5cc(-c6ccccc6)ccc5c34)c3ccccc23)c1[2H]]

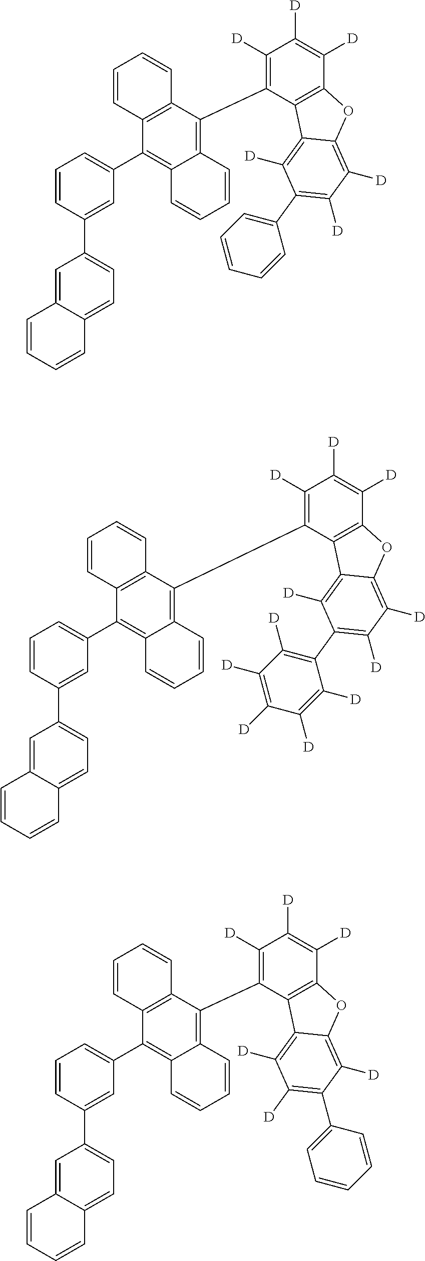 [SMILES: [2H]c1c(-c2ccccc2)c([2H])c2c(oc3c([2H])c([2H])c([2H])c(-c4c5ccccc5c(-c5cccc(-c6ccc7ccccc7c6)c5)c5ccccc45)c32)c1[2H] ; [2H]c1c([2H])c([2H])c(-c2c([2H])c([2H])c3oc4c([2H])c([2H])c([2H])c(-c5c6ccccc6c(-c6cccc(-c7ccc8ccccc8c7)c6)c6ccccc56)c4c3c2[2H])c([2H])c1[2H] ; [2H]c1c([2H])c(-c2c3ccccc3c(-c3cccc(-c4ccc5ccccc5c4)c3)c3ccccc23)c2c(oc3c([2H])c(-c4ccccc4)c([2H])c([2H])c32)c1[2H]]

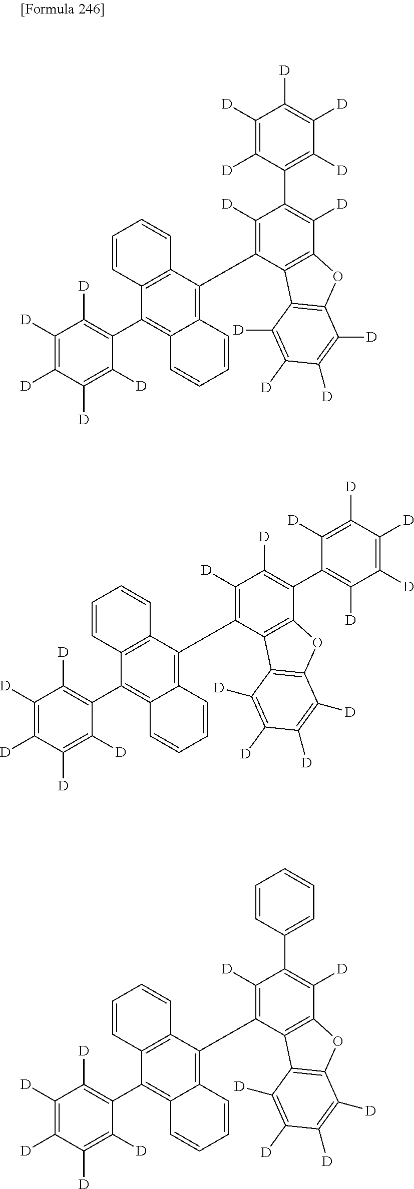 [SMILES: [2H]c1c([2H])c([2H])c(-c2c([2H])c(-c3c4ccccc4c(-c4c([2H])c([2H])c([2H])c([2H])c4[2H])c4ccccc34)c3c(oc4c([2H])c([2H])c([2H])c([2H])c43)c2[2H])c([2H])c1[2H] ; [2H]c1c([2H])c([2H])c(-c2c3ccccc3c(-c3c([2H])c([2H])c(-c4c([2H])c([2H])c([2H])c([2H])c4[2H])c4oc5c([2H])c([2H])c([2H])c([2H])c5c34)c3ccccc23)c([2H])c1[2H] ; [2H]c1c([2H])c([2H])c(-c2c3ccccc3c(-c3c([2H])c(-c4ccccc4)c([2H])c4oc5c([2H])c([2H])c([2H])c([2H])c5c34)c3ccccc23)c([2H])c1[2H]]

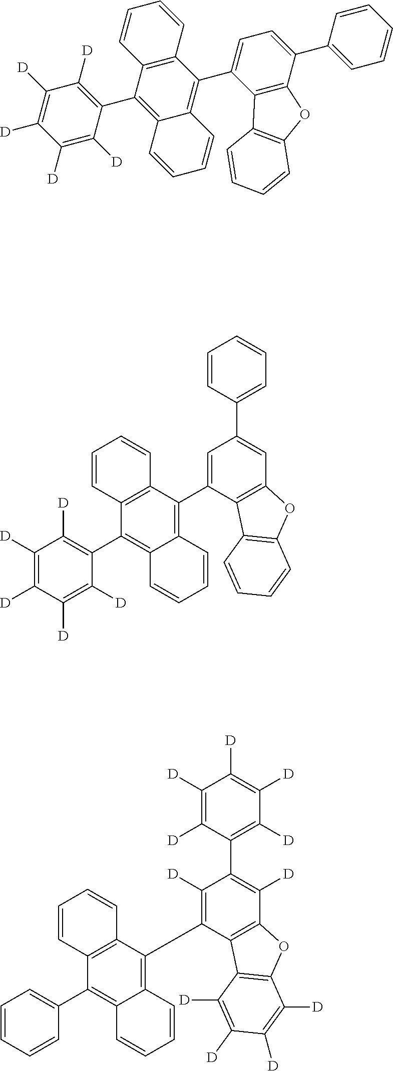 [SMILES: [2H]c1c([2H])c([2H])c(-c2c3ccccc3c(-c3ccc(-c4ccccc4)c4oc5ccccc5c34)c3ccccc23)c([2H])c1[2H] ; [2H]c1c([2H])c([2H])c(-c2c3ccccc3c(-c3cc(-c4ccccc4)cc4oc5ccccc5c34)c3ccccc23)c([2H])c1[2H] ; [2H]c1c([2H])c([2H])c(-c2c([2H])c(-c3c4ccccc4c(-c4ccccc4)c4ccccc34)c3c(oc4c([2H])c([2H])c([2H])c([2H])c43)c2[2H])c([2H])c1[2H]]

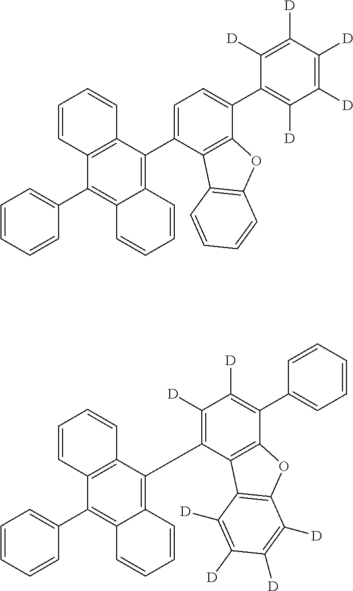 [SMILES: [2H]c1c([2H])c([2H])c(-c2ccc(-c3c4ccccc4c(-c4ccccc4)c4ccccc34)c3c2oc2ccccc23)c([2H])c1[2H] ; [2H]c1c([2H])c([2H])c2c(oc3c(-c4ccccc4)c([2H])c([2H])c(-c4c5ccccc5c(-c5ccccc5)c5ccccc45)c32)c1[2H]]

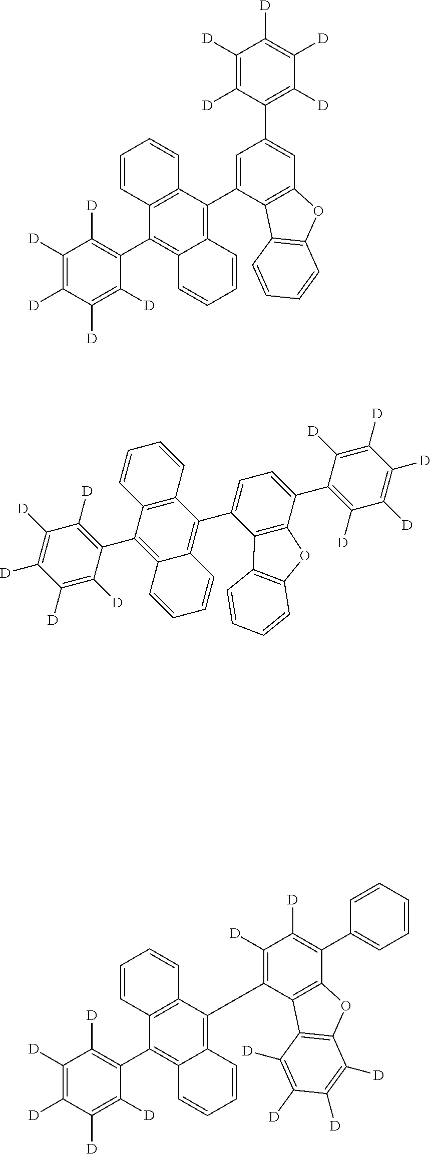 [SMILES: [2H]c1c([2H])c([2H])c(-c2cc(-c3c4ccccc4c(-c4c([2H])c([2H])c([2H])c([2H])c4[2H])c4ccccc34)c3c(c2)oc2ccccc23)c([2H])c1[2H] ; [2H]c1c([2H])c([2H])c(-c2c3ccccc3c(-c3ccc(-c4c([2H])c([2H])c([2H])c([2H])c4[2H])c4oc5ccccc5c34)c3ccccc23)c([2H])c1[2H] ; [2H]c1c([2H])c([2H])c(-c2c3ccccc3c(-c3c([2H])c([2H])c(-c4ccccc4)c4oc5c([2H])c([2H])c([2H])c([2H])c5c34)c3ccccc23)c([2H])c1[2H]]

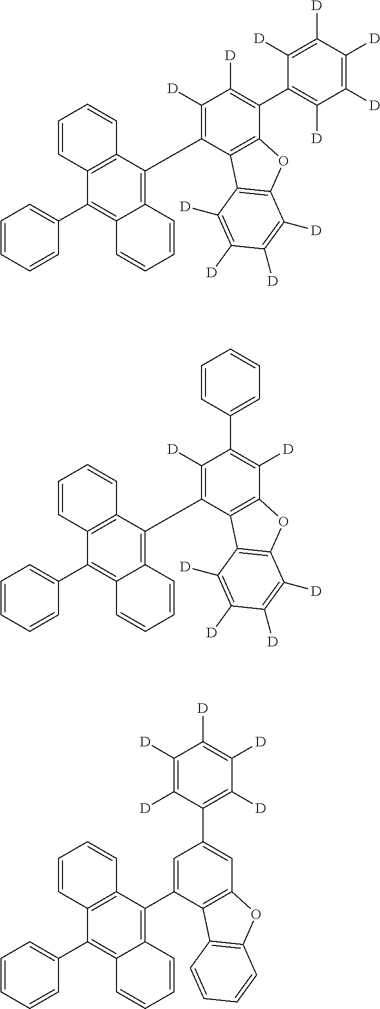 [SMILES: [2H]c1c([2H])c([2H])c(-c2c([2H])c([2H])c(-c3c4ccccc4c(-c4ccccc4)c4ccccc34)c3c2oc2c([2H])c([2H])c([2H])c([2H])c23)c([2H])c1[2H] ; [2H]c1c([2H])c([2H])c2c(oc3c([2H])c(-c4ccccc4)c([2H])c(-c4c5ccccc5c(-c5ccccc5)c5ccccc45)c32)c1[2H] ; [2H]c1c([2H])c([2H])c(-c2cc(-c3c4ccccc4c(-c4ccccc4)c4ccccc34)c3c(c2)oc2ccccc23)c([2H])c1[2H]]

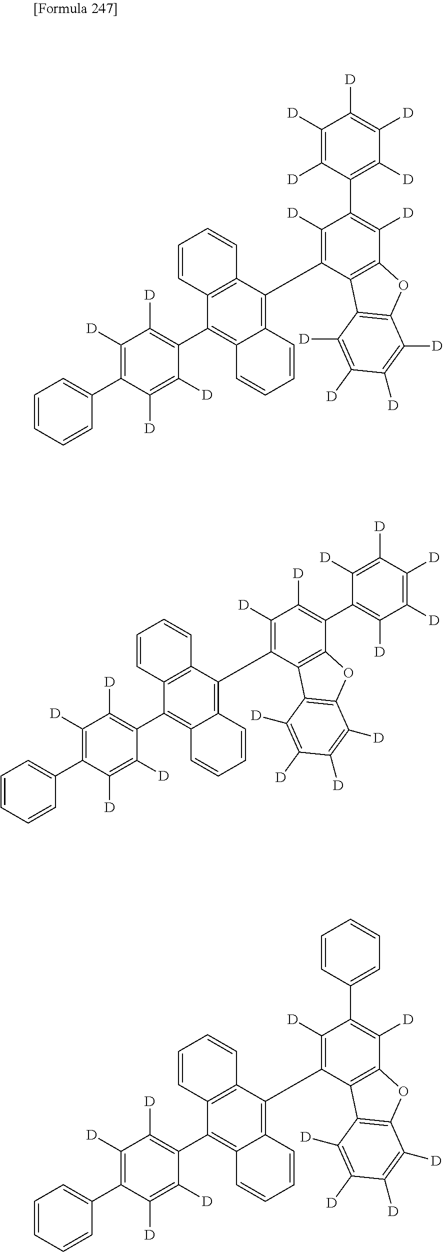 [SMILES: [2H]c1c([2H])c([2H])c(-c2c([2H])c(-c3c4ccccc4c(-c4c([2H])c([2H])c(-c5ccccc5)c([2H])c4[2H])c4ccccc34)c3c(oc4c([2H])c([2H])c([2H])c([2H])c43)c2[2H])c([2H])c1[2H] ; [2H]c1c([2H])c([2H])c(-c2c([2H])c([2H])c(-c3c4ccccc4c(-c4c([2H])c([2H])c(-c5ccccc5)c([2H])c4[2H])c4ccccc34)c3c2oc2c([2H])c([2H])c([2H])c([2H])c23)c([2H])c1[2H] ; [2H]c1c([2H])c(-c2c3ccccc3c(-c3c([2H])c(-c4ccccc4)c([2H])c4oc5c([2H])c([2H])c([2H])c([2H])c5c34)c3ccccc23)c([2H])c([2H])c1-c1ccccc1]

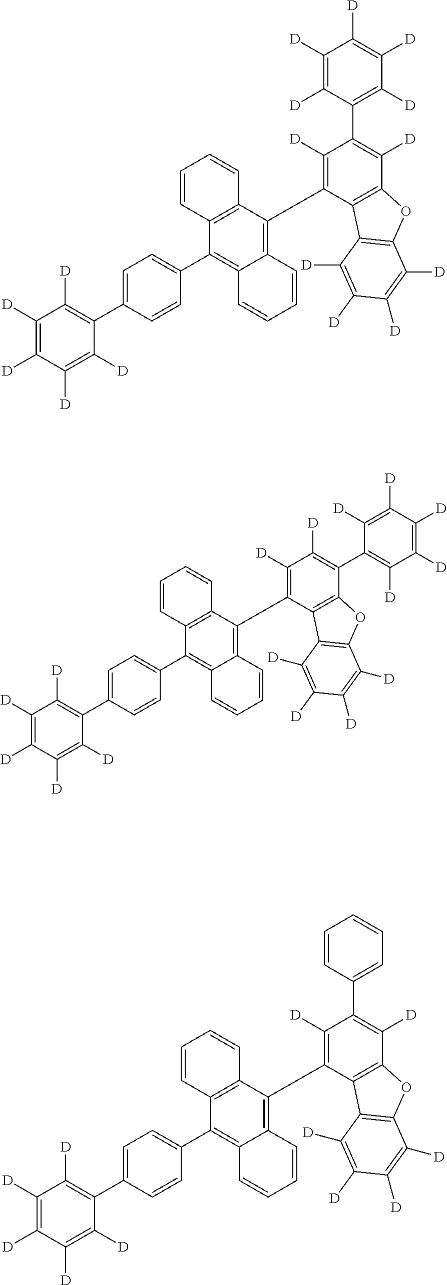 [SMILES: [2H]c1c([2H])c([2H])c(-c2ccc(-c3c4ccccc4c(-c4c([2H])c(-c5c([2H])c([2H])c([2H])c([2H])c5[2H])c([2H])c5oc6c([2H])c([2H])c([2H])c([2H])c6c45)c4ccccc34)cc2)c([2H])c1[2H] ; [2H]c1c([2H])c([2H])c(-c2ccc(-c3c4ccccc4c(-c4c([2H])c([2H])c(-c5c([2H])c([2H])c([2H])c([2H])c5[2H])c5oc6c([2H])c([2H])c([2H])c([2H])c6c45)c4ccccc34)cc2)c([2H])c1[2H] ; [2H]c1c([2H])c([2H])c(-c2ccc(-c3c4ccccc4c(-c4c([2H])c(-c5ccccc5)c([2H])c5oc6c([2H])c([2H])c([2H])c([2H])c6c45)c4ccccc34)cc2)c([2H])c1[2H]]

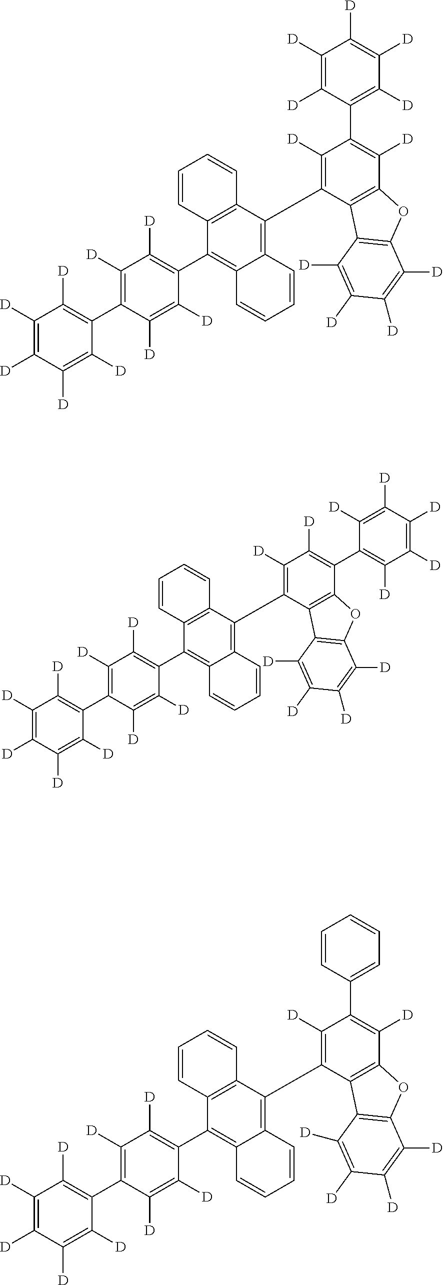

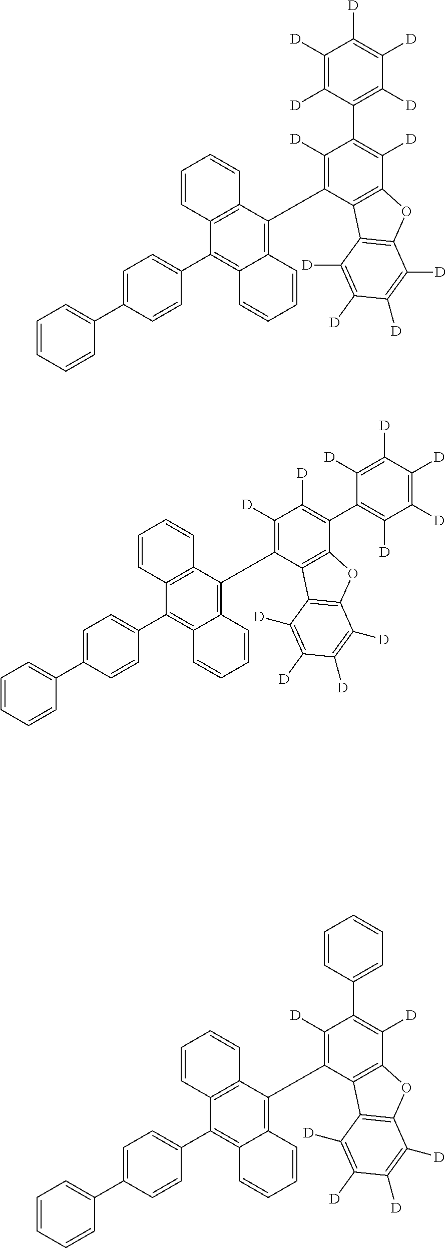 [SMILES: [2H]c1c([2H])c([2H])c(-c2c([2H])c(-c3c4ccccc4c(-c4ccc(-c5ccccc5)cc4)c4ccccc34)c3c(oc4c([2H])c([2H])c([2H])c([2H])c43)c2[2H])c([2H])c1[2H] ; [2H]c1c([2H])c([2H])c(-c2c([2H])c([2H])c(-c3c4ccccc4c(-c4ccc(-c5ccccc5)cc4)c4ccccc34)c3c2oc2c([2H])c([2H])c([2H])c([2H])c23)c([2H])c1[2H] ; [2H]c1c([2H])c([2H])c2c(oc3c([2H])c(-c4ccccc4)c([2H])c(-c4c5ccccc5c(-c5ccc(-c6ccccc6)cc5)c5ccccc45)c32)c1[2H]]

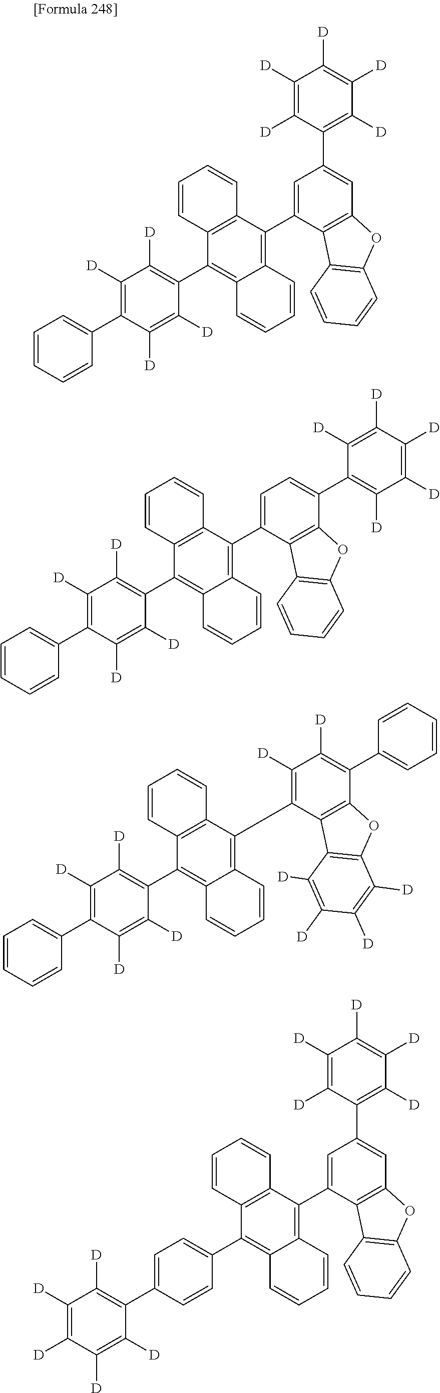

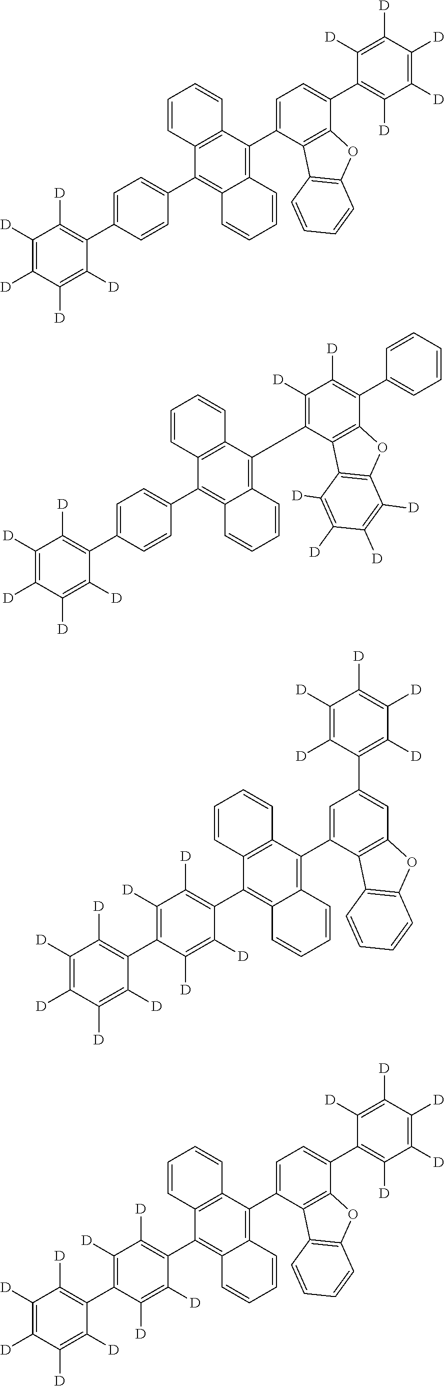

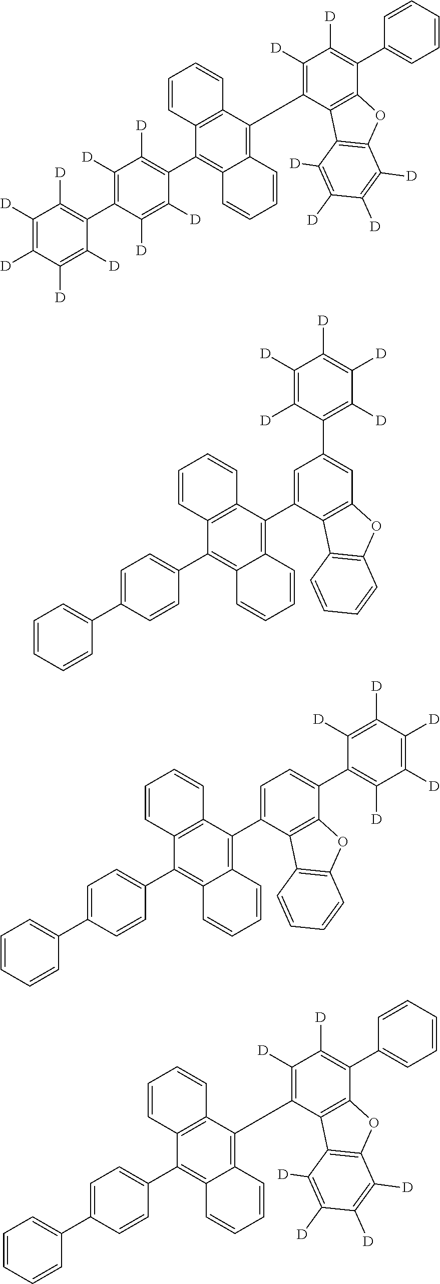 [SMILES: [2H]c1c([2H])c([2H])c(-c2c([2H])c([2H])c(-c3c4ccccc4c(-c4c([2H])c([2H])c(-c5ccccc5)c5oc6c([2H])c([2H])c([2H])c([2H])c6c45)c4ccccc34)c([2H])c2[2H])c([2H])c1[2H] ; [2H]c1c([2H])c([2H])c(-c2cc(-c3c4ccccc4c(-c4ccc(-c5ccccc5)cc4)c4ccccc34)c3c(c2)oc2ccccc23)c([2H])c1[2H] ; [2H]c1c([2H])c([2H])c(-c2ccc(-c3c4ccccc4c(-c4ccc(-c5ccccc5)cc4)c4ccccc34)c3c2oc2ccccc23)c([2H])c1[2H] ; [2H]c1c([2H])c([2H])c2c(oc3c(-c4ccccc4)c([2H])c([2H])c(-c4c5ccccc5c(-c5ccc(-c6ccccc6)cc5)c5ccccc45)c32)c1[2H]]

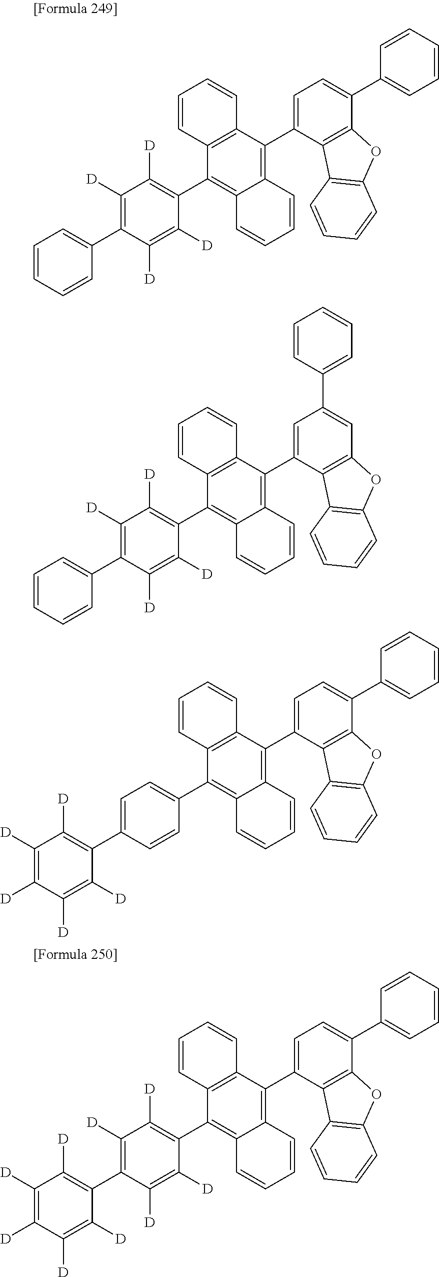 [SMILES: [2H]c1c([2H])c(-c2c3ccccc3c(-c3ccc(-c4ccccc4)c4oc5ccccc5c34)c3ccccc23)c([2H])c([2H])c1-c1ccccc1 ; [2H]c1c([2H])c(-c2c3ccccc3c(-c3cc(-c4ccccc4)cc4oc5ccccc5c34)c3ccccc23)c([2H])c([2H])c1-c1ccccc1 ; [2H]c1c([2H])c([2H])c(-c2ccc(-c3c4ccccc4c(-c4ccc(-c5ccccc5)c5oc6ccccc6c45)c4ccccc34)cc2)c([2H])c1[2H] ; [2H]c1c([2H])c([2H])c(-c2c([2H])c([2H])c(-c3c4ccccc4c(-c4ccc(-c5ccccc5)c5oc6ccccc6c45)c4ccccc34)c([2H])c2[2H])c([2H])c1[2H]]

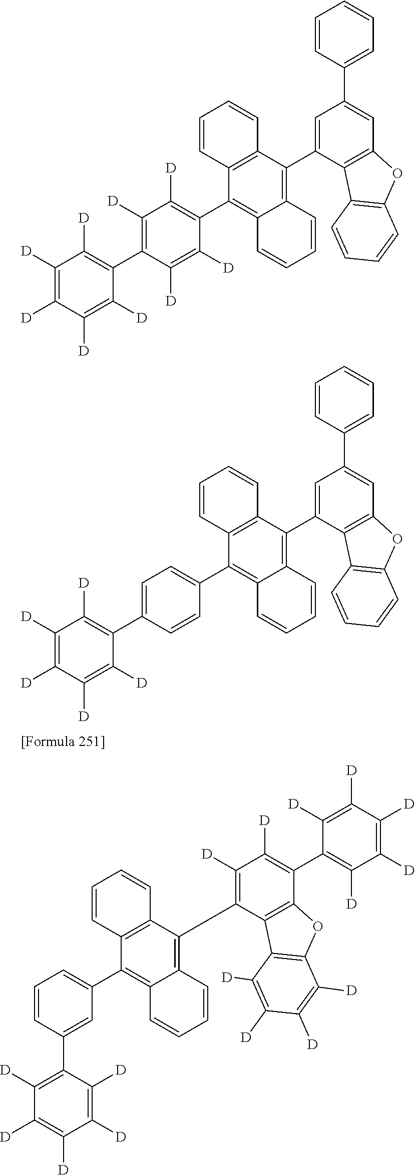 [SMILES: [2H]c1c([2H])c([2H])c(-c2c([2H])c([2H])c(-c3c4ccccc4c(-c4cc(-c5ccccc5)cc5oc6ccccc6c45)c4ccccc34)c([2H])c2[2H])c([2H])c1[2H] ; [2H]c1c([2H])c([2H])c(-c2ccc(-c3c4ccccc4c(-c4cc(-c5ccccc5)cc5oc6ccccc6c45)c4ccccc34)cc2)c([2H])c1[2H] ; [2H]c1c([2H])c([2H])c(-c2cccc(-c3c4ccccc4c(-c4c([2H])c([2H])c(-c5c([2H])c([2H])c([2H])c([2H])c5[2H])c5oc6c([2H])c([2H])c([2H])c([2H])c6c45)c4ccccc34)c2)c([2H])c1[2H]]

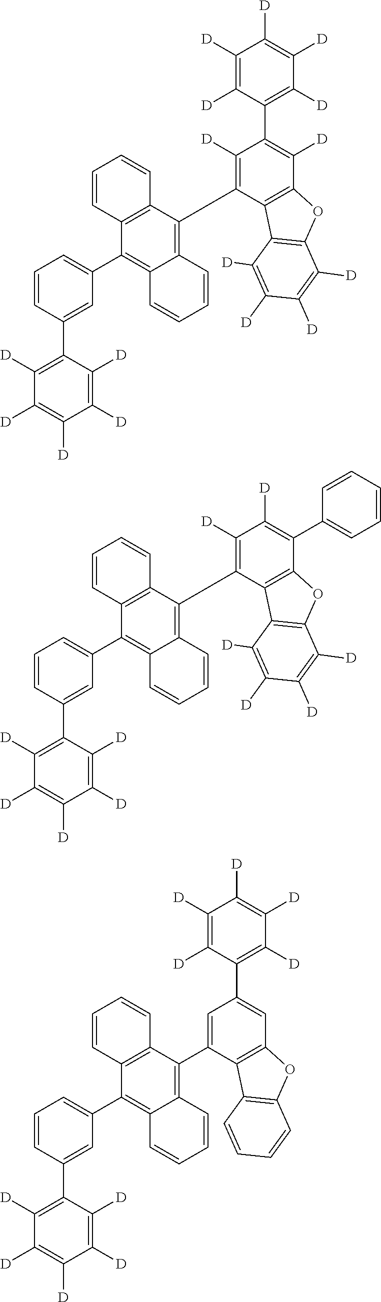 [SMILES: [2H]c1c([2H])c([2H])c(-c2cccc(-c3c4ccccc4c(-c4c([2H])c(-c5c([2H])c([2H])c([2H])c([2H])c5[2H])c([2H])c5oc6c([2H])c([2H])c([2H])c([2H])c6c45)c4ccccc34)c2)c([2H])c1[2H] ; [2H]c1c([2H])c([2H])c(-c2cccc(-c3c4ccccc4c(-c4c([2H])c([2H])c(-c5ccccc5)c5oc6c([2H])c([2H])c([2H])c([2H])c6c45)c4ccccc34)c2)c([2H])c1[2H] ; [2H]c1c([2H])c([2H])c(-c2cccc(-c3c4ccccc4c(-c4cc(-c5c([2H])c([2H])c([2H])c([2H])c5[2H])cc5oc6ccccc6c45)c4ccccc34)c2)c([2H])c1[2H]]

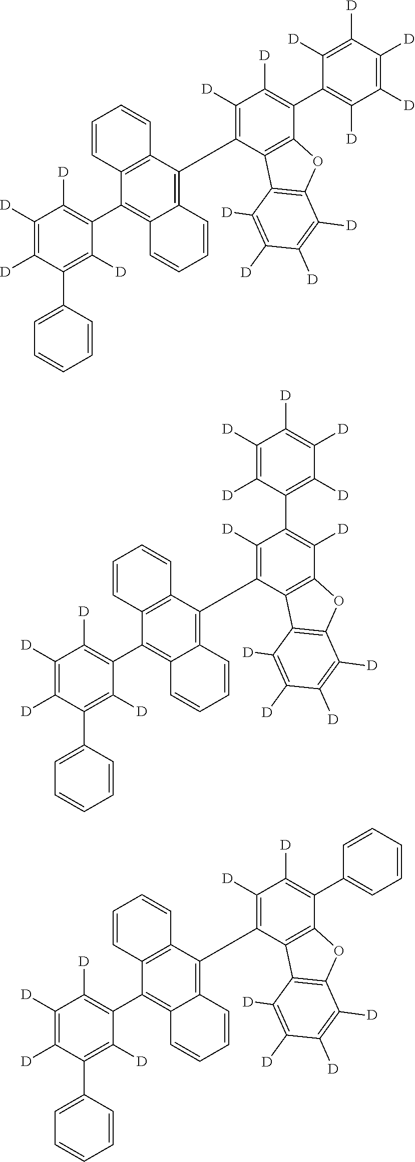 [SMILES: [2H]c1c([2H])c([2H])c(-c2c([2H])c([2H])c(-c3c4ccccc4c(-c4c([2H])c([2H])c([2H])c(-c5ccccc5)c4[2H])c4ccccc34)c3c2oc2c([2H])c([2H])c([2H])c([2H])c23)c([2H])c1[2H] ; [2H]c1c([2H])c([2H])c(-c2c([2H])c(-c3c4ccccc4c(-c4c([2H])c([2H])c([2H])c(-c5ccccc5)c4[2H])c4ccccc34)c3c(oc4c([2H])c([2H])c([2H])c([2H])c43)c2[2H])c([2H])c1[2H] ; [2H]c1c([2H])c(-c2ccccc2)c([2H])c(-c2c3ccccc3c(-c3c([2H])c([2H])c(-c4ccccc4)c4oc5c([2H])c([2H])c([2H])c([2H])c5c34)c3ccccc23)c1[2H]]

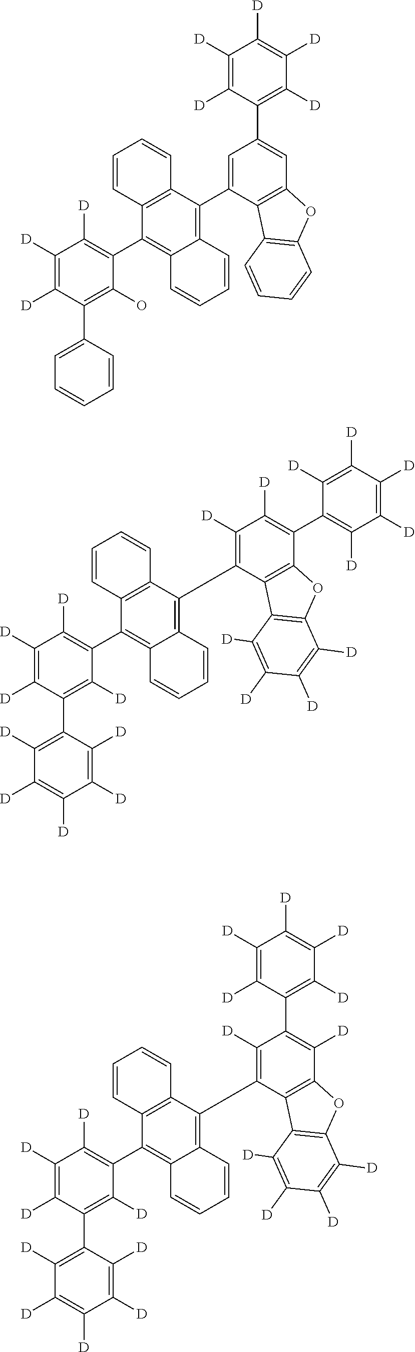

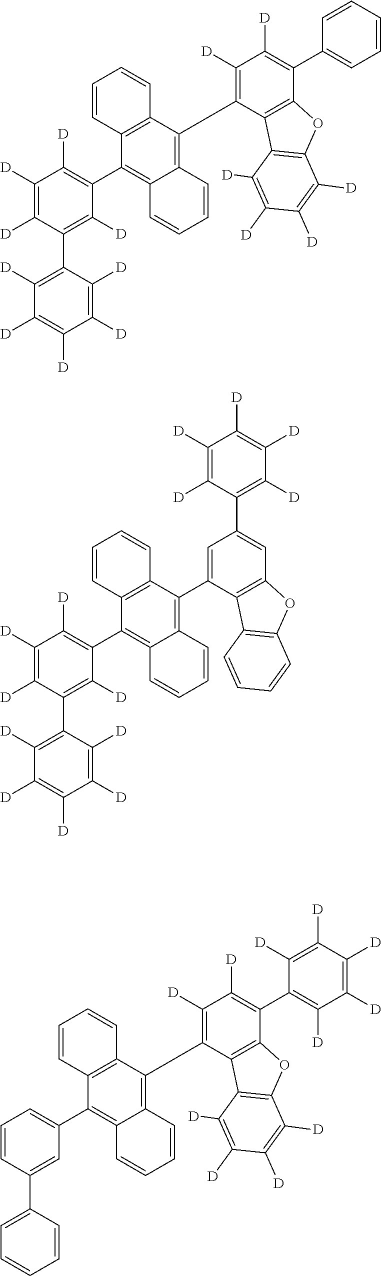 [SMILES: [2H]c1c([2H])c([2H])c(-c2c([2H])c([2H])c([2H])c(-c3c4ccccc4c(-c4c([2H])c([2H])c(-c5ccccc5)c5oc6c([2H])c([2H])c([2H])c([2H])c6c45)c4ccccc34)c2[2H])c([2H])c1[2H] ; [2H]c1c([2H])c([2H])c(-c2cc(-c3c4ccccc4c(-c4c([2H])c([2H])c([2H])c(-c5c([2H])c([2H])c([2H])c([2H])c5[2H])c4[2H])c4ccccc34)c3c(c2)oc2ccccc23)c([2H])c1[2H] ; [2H]c1c([2H])c([2H])c(-c2c([2H])c([2H])c(-c3c4ccccc4c(-c4cccc(-c5ccccc5)c4)c4ccccc34)c3c2oc2c([2H])c([2H])c([2H])c([2H])c23)c([2H])c1[2H]]

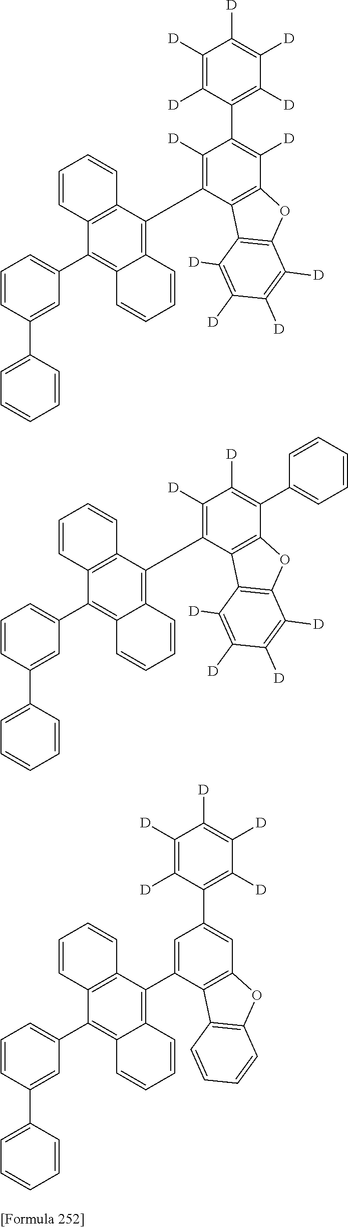 [SMILES: [2H]c1c([2H])c([2H])c(-c2c([2H])c(-c3c4ccccc4c(-c4cccc(-c5ccccc5)c4)c4ccccc34)c3c(oc4c([2H])c([2H])c([2H])c([2H])c43)c2[2H])c([2H])c1[2H] ; [2H]c1c([2H])c([2H])c2c(oc3c(-c4ccccc4)c([2H])c([2H])c(-c4c5ccccc5c(-c5cccc(-c6ccccc6)c5)c5ccccc45)c32)c1[2H] ; [2H]c1c([2H])c([2H])c(-c2cc(-c3c4ccccc4c(-c4cccc(-c5ccccc5)c4)c4ccccc34)c3c(c2)oc2ccccc23)c([2H])c1[2H]]

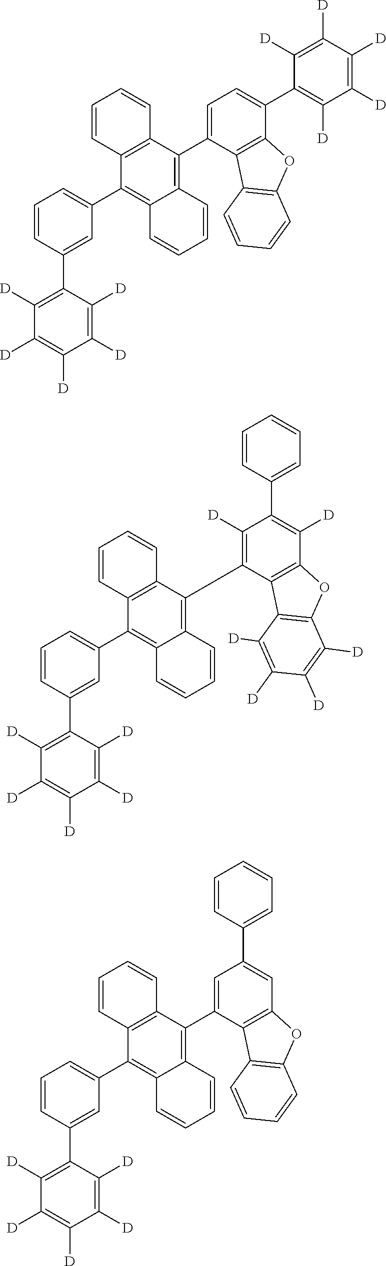 [SMILES: [2H]c1c([2H])c([2H])c(-c2cccc(-c3c4ccccc4c(-c4ccc(-c5c([2H])c([2H])c([2H])c([2H])c5[2H])c5oc6ccccc6c45)c4ccccc34)c2)c([2H])c1[2H] ; [2H]c1c([2H])c([2H])c(-c2cccc(-c3c4ccccc4c(-c4c([2H])c(-c5ccccc5)c([2H])c5oc6c([2H])c([2H])c([2H])c([2H])c6c45)c4ccccc34)c2)c([2H])c1[2H] ; [2H]c1c([2H])c([2H])c(-c2cccc(-c3c4ccccc4c(-c4cc(-c5ccccc5)cc5oc6ccccc6c45)c4ccccc34)c2)c([2H])c1[2H]]

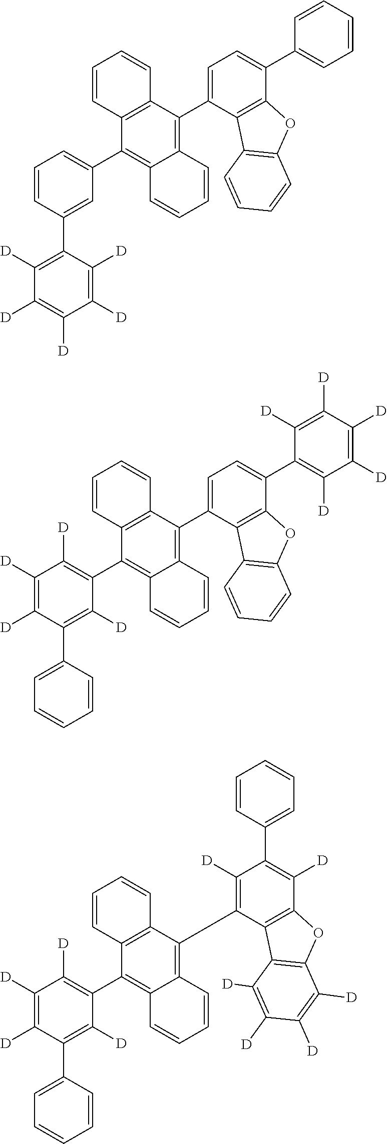 [SMILES: [2H]c1c([2H])c([2H])c(-c2cccc(-c3c4ccccc4c(-c4ccc(-c5ccccc5)c5oc6ccccc6c45)c4ccccc34)c2)c([2H])c1[2H] ; [2H]c1c([2H])c([2H])c(-c2ccc(-c3c4ccccc4c(-c4c([2H])c([2H])c([2H])c(-c5ccccc5)c4[2H])c4ccccc34)c3c2oc2ccccc23)c([2H])c1[2H] ; [2H]c1c([2H])c(-c2ccccc2)c([2H])c(-c2c3ccccc3c(-c3c([2H])c(-c4ccccc4)c([2H])c4oc5c([2H])c([2H])c([2H])c([2H])c5c34)c3ccccc23)c1[2H]]

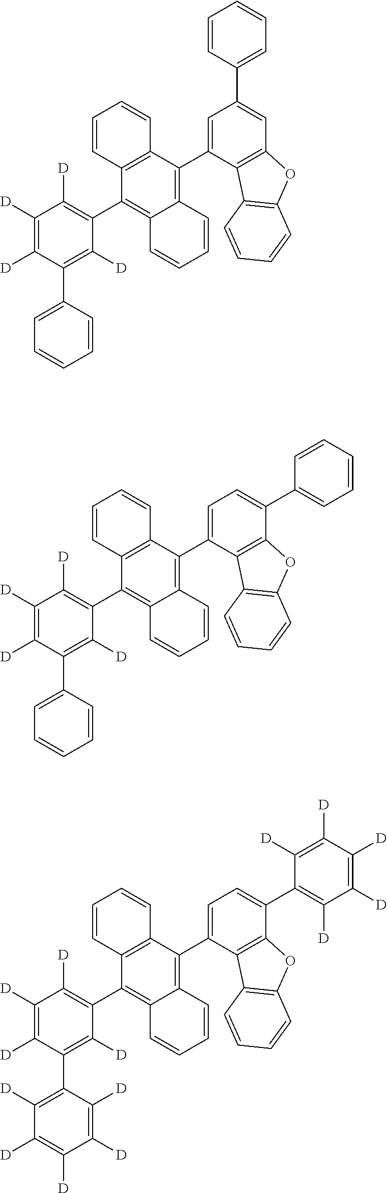 [SMILES: [2H]c1c([2H])c(-c2ccccc2)c([2H])c(-c2c3ccccc3c(-c3cc(-c4ccccc4)cc4oc5ccccc5c34)c3ccccc23)c1[2H] ; [2H]c1c([2H])c(-c2ccccc2)c([2H])c(-c2c3ccccc3c(-c3ccc(-c4ccccc4)c4oc5ccccc5c34)c3ccccc23)c1[2H] ; [2H]c1c([2H])c([2H])c(-c2c([2H])c([2H])c([2H])c(-c3c4ccccc4c(-c4ccc(-c5c([2H])c([2H])c([2H])c([2H])c5[2H])c5oc6ccccc6c45)c4ccccc34)c2[2H])c([2H])c1[2H]]

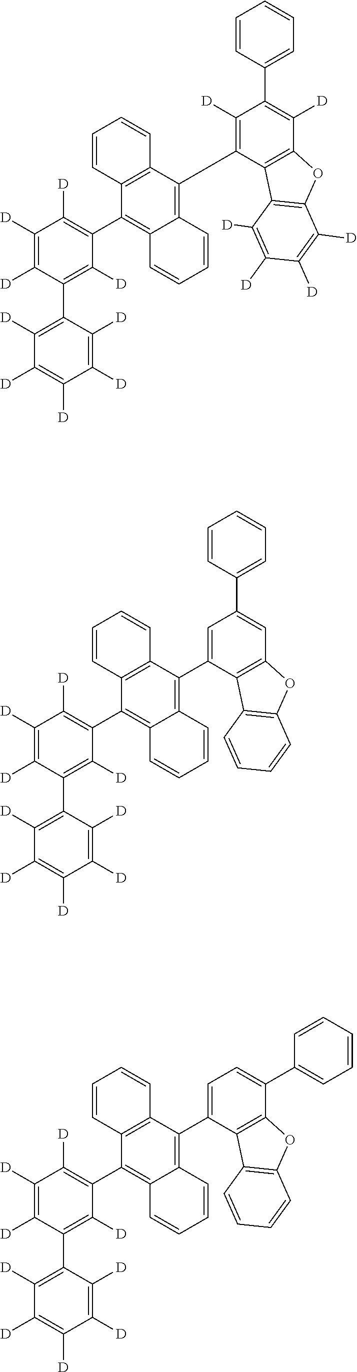 [SMILES: [2H]c1c([2H])c([2H])c(-c2c([2H])c([2H])c([2H])c(-c3c4ccccc4c(-c4c([2H])c(-c5ccccc5)c([2H])c5oc6c([2H])c([2H])c([2H])c([2H])c6c45)c4ccccc34)c2[2H])c([2H])c1[2H] ; [2H]c1c([2H])c([2H])c(-c2c([2H])c([2H])c([2H])c(-c3c4ccccc4c(-c4cc(-c5ccccc5)cc5oc6ccccc6c45)c4ccccc34)c2[2H])c([2H])c1[2H] ; [2H]c1c([2H])c([2H])c(-c2c([2H])c([2H])c([2H])c(-c3c4ccccc4c(-c4ccc(-c5ccccc5)c5oc6ccccc6c45)c4ccccc34)c2[2H])c([2H])c1[2H]]

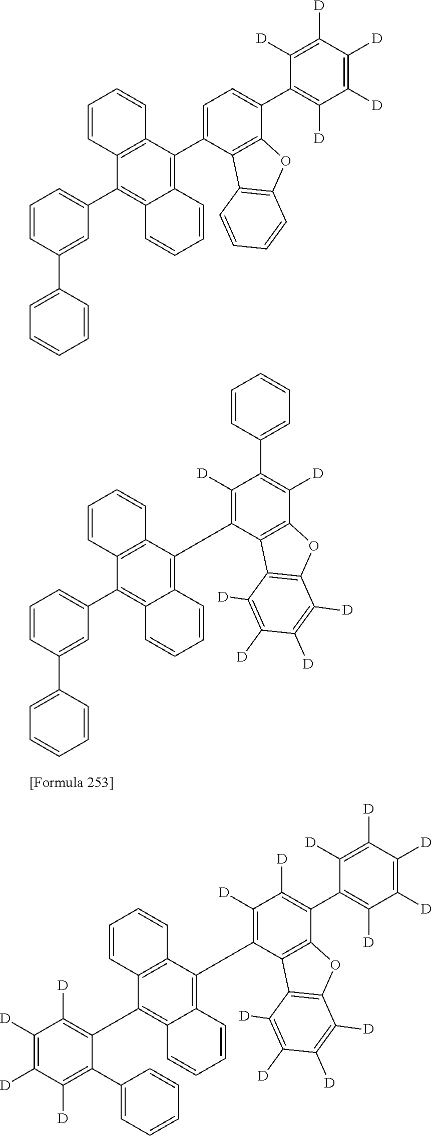 [SMILES: [2H]c1c([2H])c([2H])c(-c2ccc(-c3c4ccccc4c(-c4cccc(-c5ccccc5)c4)c4ccccc34)c3c2oc2ccccc23)c([2H])c1[2H] ; [2H]c1c([2H])c([2H])c2c(oc3c([2H])c(-c4ccccc4)c([2H])c(-c4c5ccccc5c(-c5cccc(-c6ccccc6)c5)c5ccccc45)c32)c1[2H] ; [2H]c1c([2H])c([2H])c(-c2c([2H])c([2H])c(-c3c4ccccc4c(-c4c([2H])c([2H])c([2H])c([2H])c4-c4ccccc4)c4ccccc34)c3c2oc2c([2H])c([2H])c([2H])c([2H])c23)c([2H])c1[2H]]

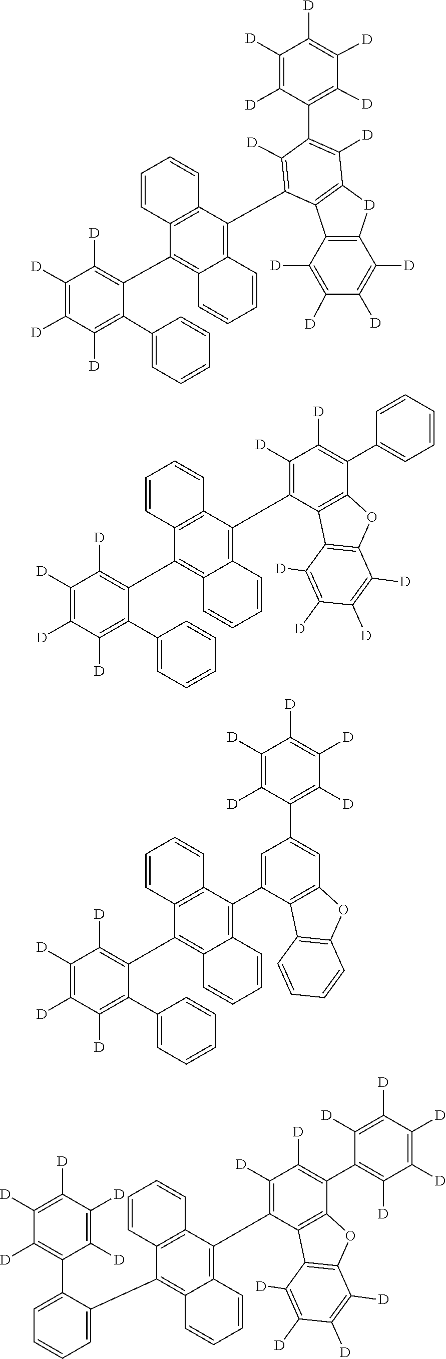

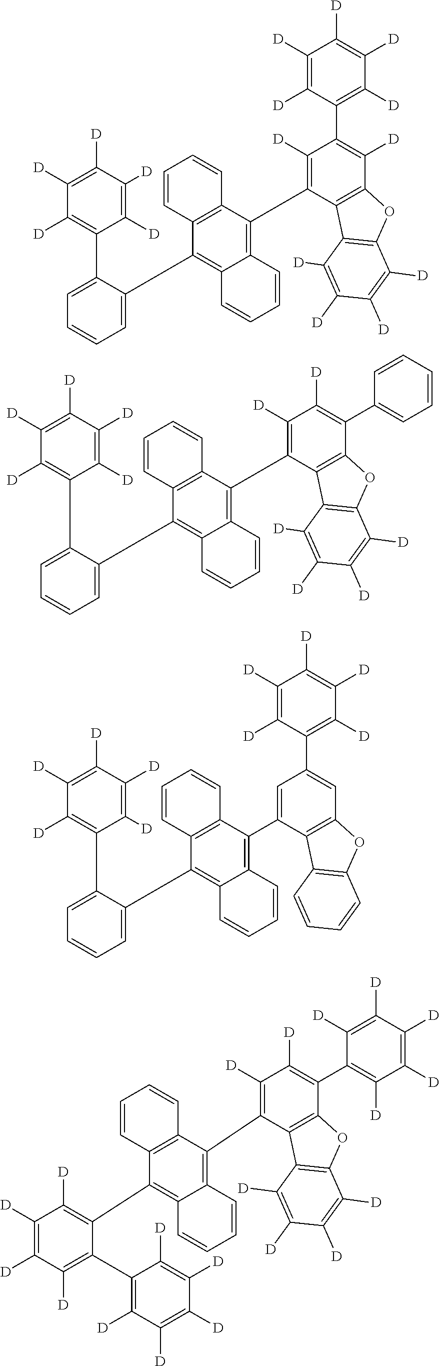

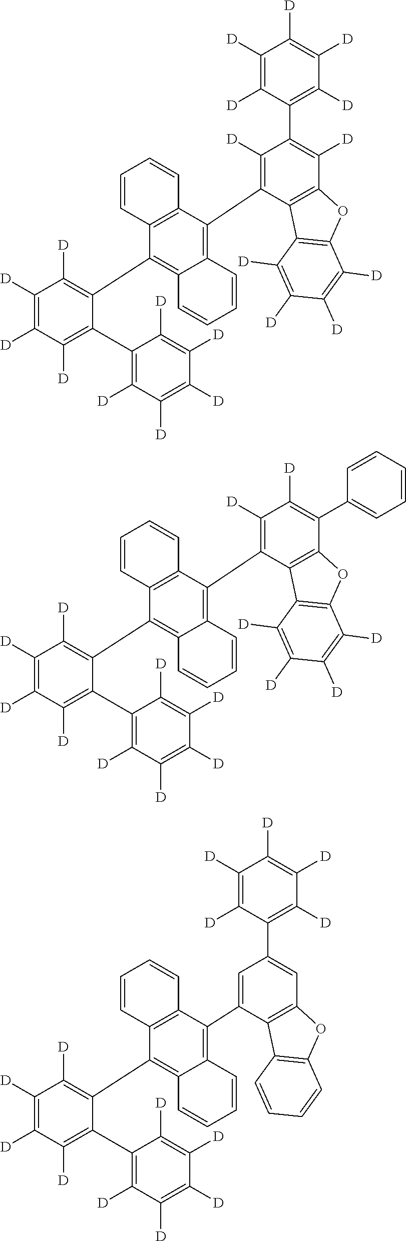

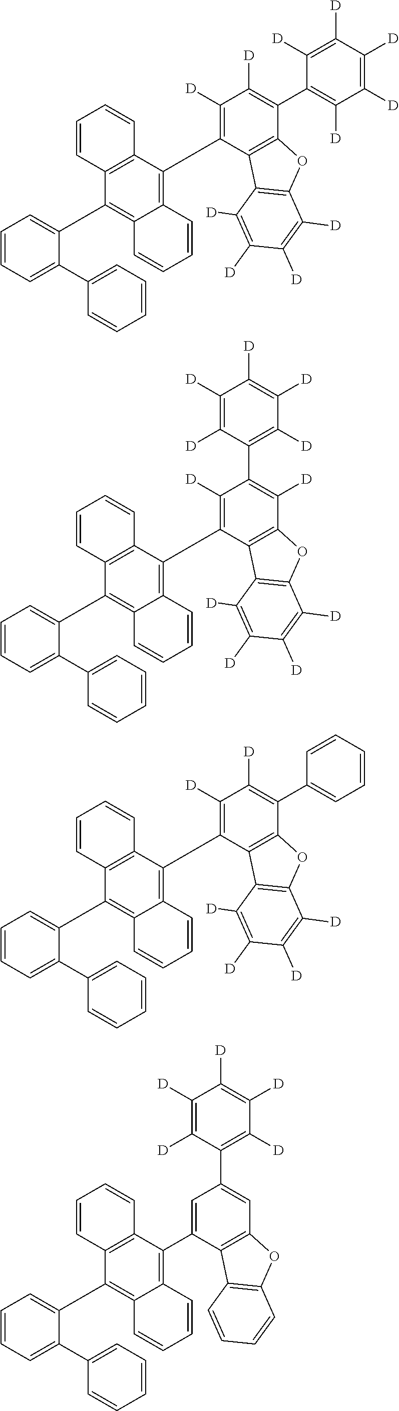 [SMILES: [2H]c1c([2H])c([2H])c(-c2c([2H])c([2H])c(-c3c4ccccc4c(-c4ccccc4-c4ccccc4)c4ccccc34)c3c2oc2c([2H])c([2H])c([2H])c([2H])c23)c([2H])c1[2H] ; [2H]c1c([2H])c([2H])c(-c2c([2H])c(-c3c4ccccc4c(-c4ccccc4-c4ccccc4)c4ccccc34)c3c(oc4c([2H])c([2H])c([2H])c([2H])c43)c2[2H])c([2H])c1[2H] ; [2H]c1c([2H])c([2H])c2c(oc3c(-c4ccccc4)c([2H])c([2H])c(-c4c5ccccc5c(-c5ccccc5-c5ccccc5)c5ccccc45)c32)c1[2H] ; [2H]c1c([2H])c([2H])c(-c2cc(-c3c4ccccc4c(-c4ccccc4-c4ccccc4)c4ccccc34)c3c(c2)oc2ccccc23)c([2H])c1[2H]]

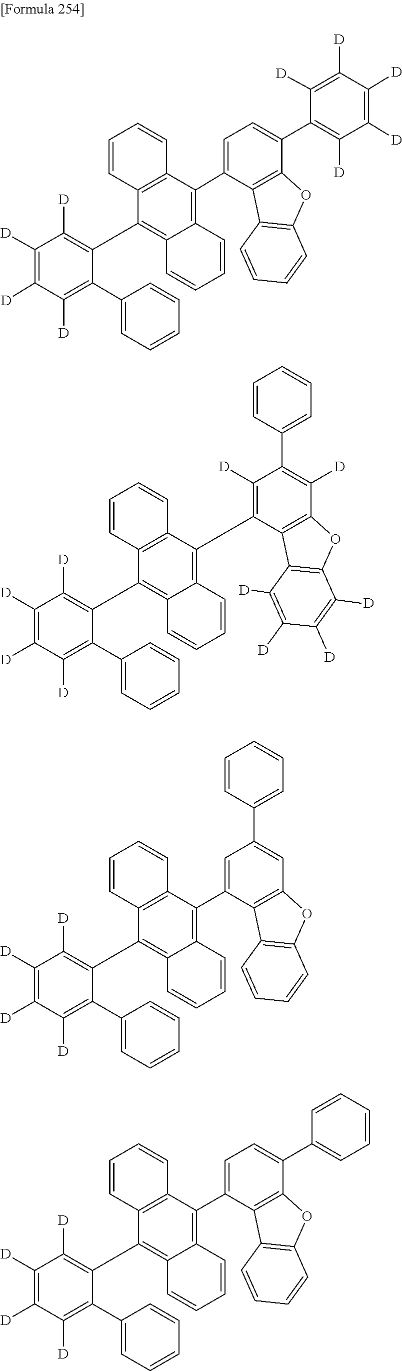 [SMILES: [2H]c1c([2H])c([2H])c(-c2ccc(-c3c4ccccc4c(-c4c([2H])c([2H])c([2H])c([2H])c4-c4ccccc4)c4ccccc34)c3c2oc2ccccc23)c([2H])c1[2H] ; [2H]c1c([2H])c([2H])c(-c2c3ccccc3c(-c3c([2H])c(-c4ccccc4)c([2H])c4oc5c([2H])c([2H])c([2H])c([2H])c5c34)c3ccccc23)c(-c2ccccc2)c1[2H] ; [2H]c1c([2H])c([2H])c(-c2c3ccccc3c(-c3cc(-c4ccccc4)cc4oc5ccccc5c34)c3ccccc23)c(-c2ccccc2)c1[2H] ; [2H]c1c([2H])c([2H])c(-c2c3ccccc3c(-c3ccc(-c4ccccc4)c4oc5ccccc5c34)c3ccccc23)c(-c2ccccc2)c1[2H]]

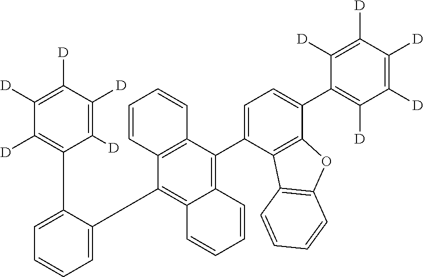 [SMILES: [2H]c1c([2H])c([2H])c(-c2ccccc2-c2c3ccccc3c(-c3ccc(-c4c([2H])c([2H])c([2H])c([2H])c4[2H])c4oc5ccccc5c34)c3ccccc23)c([2H])c1[2H]]

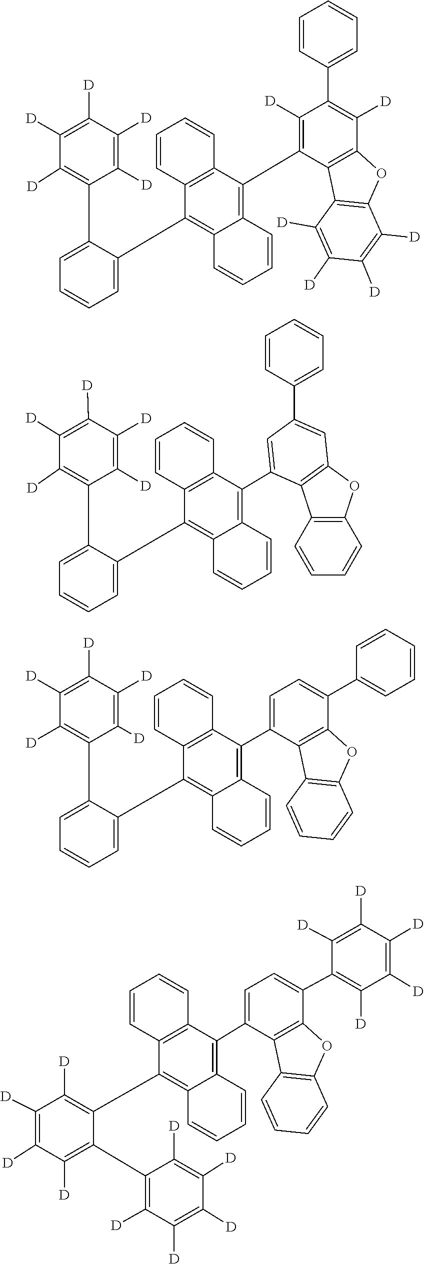

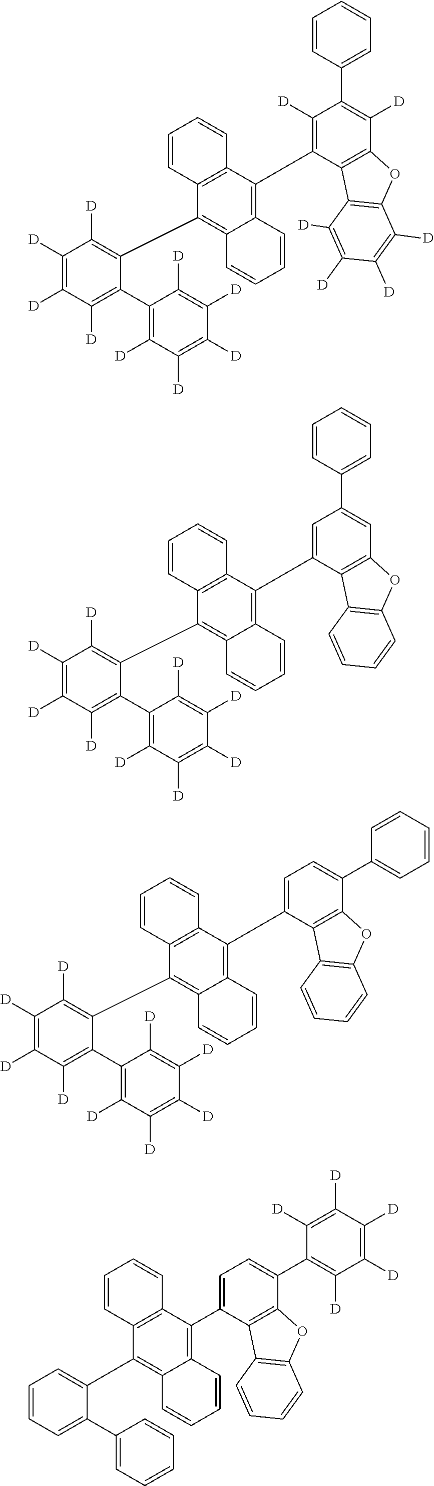

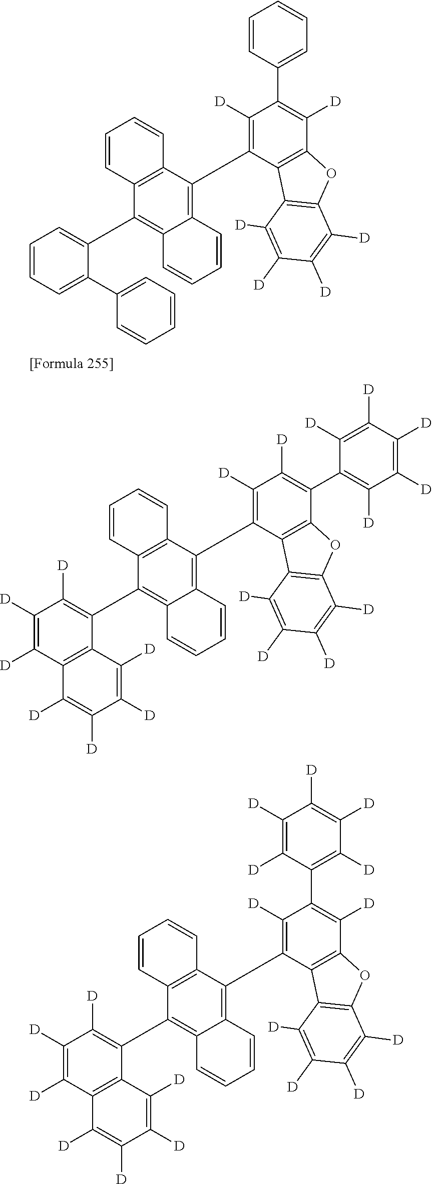 [SMILES: [2H]c1c([2H])c([2H])c2c(oc3c([2H])c(-c4ccccc4)c([2H])c(-c4c5ccccc5c(-c5ccccc5-c5ccccc5)c5ccccc45)c32)c1[2H] ; [2H]c1c([2H])c([2H])c(-c2c([2H])c([2H])c(-c3c4ccccc4c(-c4c([2H])c([2H])c([2H])c5c([2H])c([2H])c([2H])c([2H])c45)c4ccccc34)c3c2oc2c([2H])c([2H])c([2H])c([2H])c23)c([2H])c1[2H] ; [2H]c1c([2H])c([2H])c(-c2c([2H])c(-c3c4ccccc4c(-c4c([2H])c([2H])c([2H])c5c([2H])c([2H])c([2H])c([2H])c45)c4ccccc34)c3c(oc4c([2H])c([2H])c([2H])c([2H])c43)c2[2H])c([2H])c1[2H]]

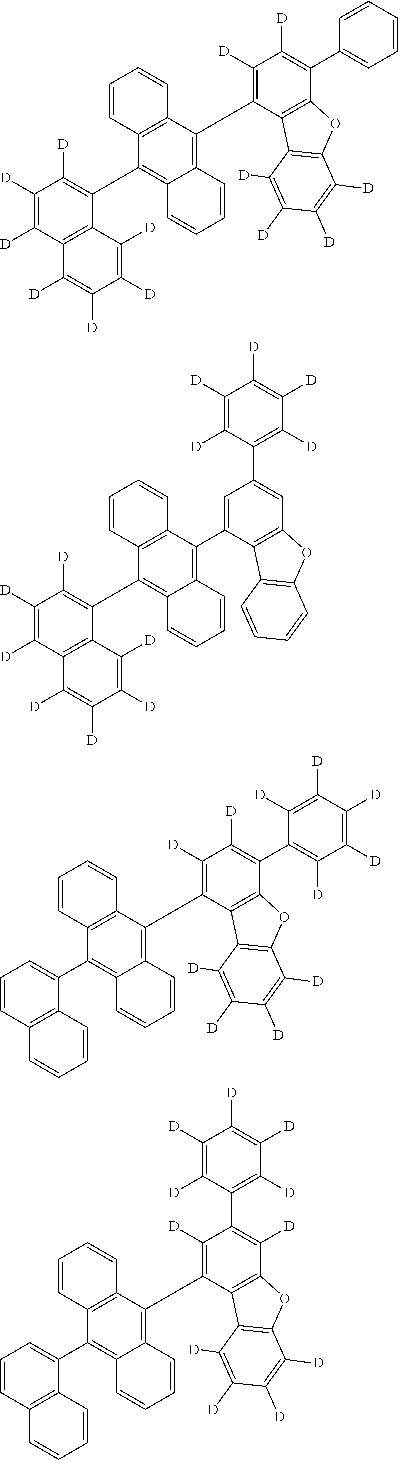

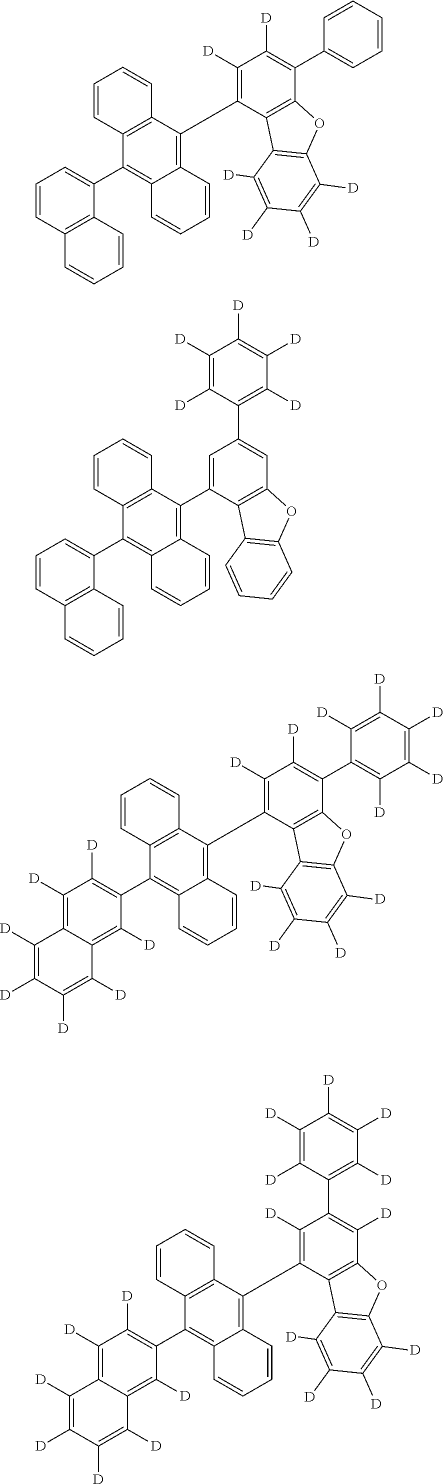

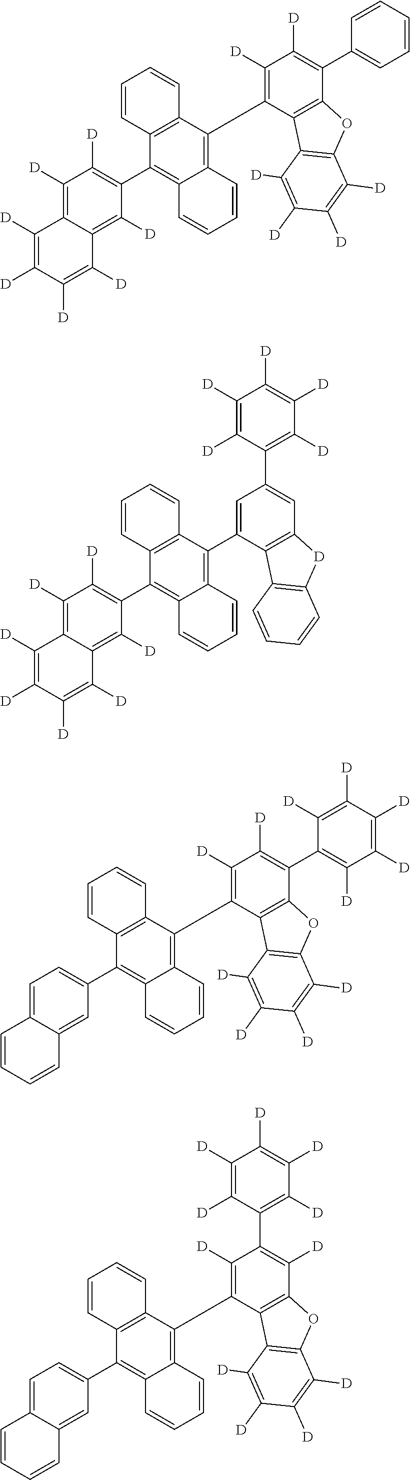

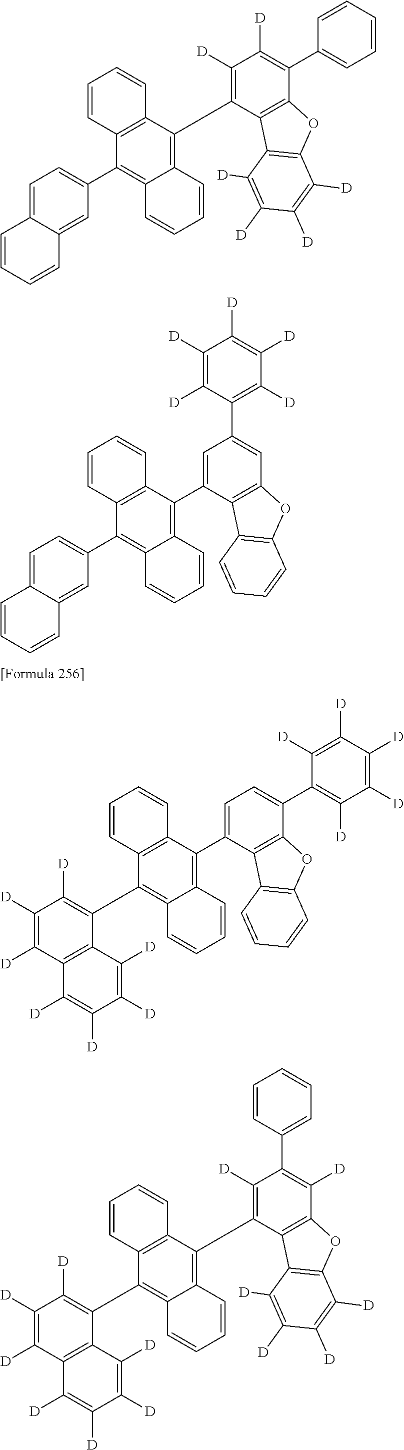 [SMILES: [2H]c1c([2H])c([2H])c2c(oc3c(-c4ccccc4)c([2H])c([2H])c(-c4c5ccccc5c(-c5ccc6ccccc6c5)c5ccccc45)c32)c1[2H] ; [2H]c1c([2H])c([2H])c(-c2cc(-c3c4ccccc4c(-c4ccc5ccccc5c4)c4ccccc34)c3c(c2)oc2ccccc23)c([2H])c1[2H] ; [2H]c1c([2H])c([2H])c(-c2ccc(-c3c4ccccc4c(-c4c([2H])c([2H])c([2H])c5c([2H])c([2H])c([2H])c([2H])c45)c4ccccc34)c3c2oc2ccccc23)c([2H])c1[2H] ; [2H]c1c([2H])c([2H])c2c(oc3c([2H])c(-c4ccccc4)c([2H])c(-c4c5ccccc5c(-c5c([2H])c([2H])c([2H])c6c([2H])c([2H])c([2H])c([2H])c56)c5ccccc45)c32)c1[2H]]

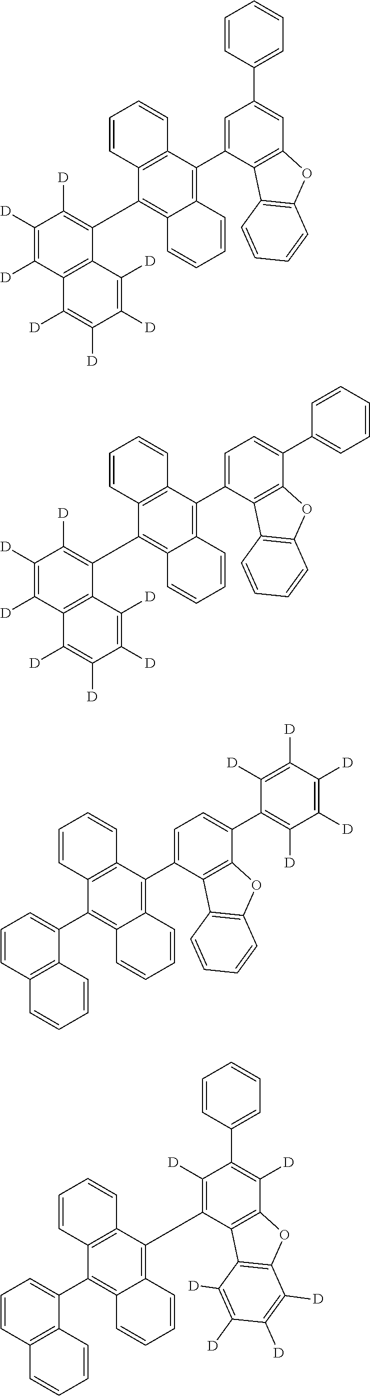 [SMILES: [2H]c1c([2H])c([2H])c2c(-c3c4ccccc4c(-c4cc(-c5ccccc5)cc5oc6ccccc6c45)c4ccccc34)c([2H])c([2H])c([2H])c2c1[2H] ; [2H]c1c([2H])c([2H])c2c(-c3c4ccccc4c(-c4ccc(-c5ccccc5)c5oc6ccccc6c45)c4ccccc34)c([2H])c([2H])c([2H])c2c1[2H] ; [2H]c1c([2H])c([2H])c(-c2ccc(-c3c4ccccc4c(-c4cccc5ccccc45)c4ccccc34)c3c2oc2ccccc23)c([2H])c1[2H] ; [2H]c1c([2H])c([2H])c2c(oc3c([2H])c(-c4ccccc4)c([2H])c(-c4c5ccccc5c(-c5cccc6ccccc56)c5ccccc45)c32)c1[2H]]

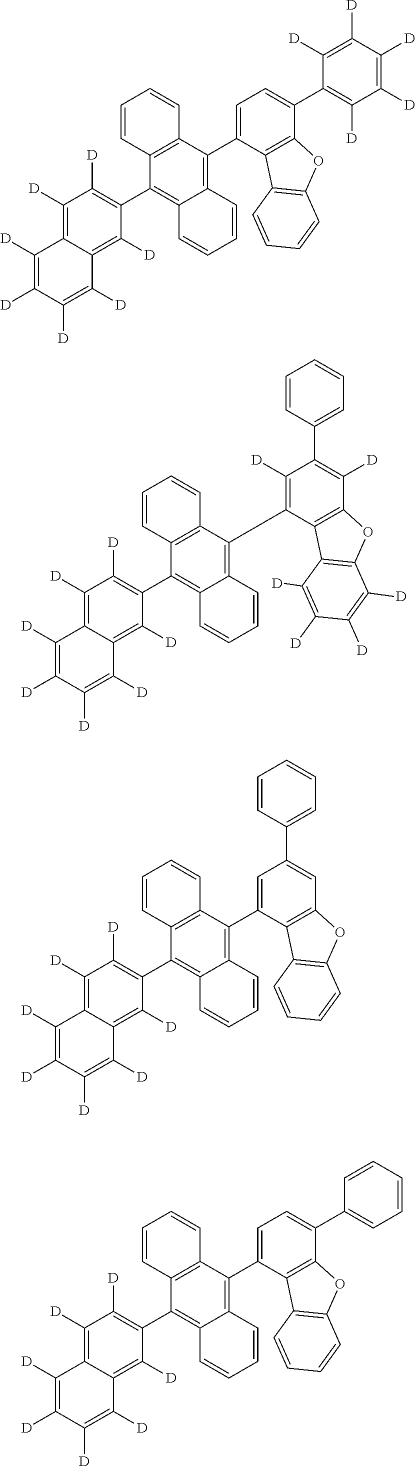

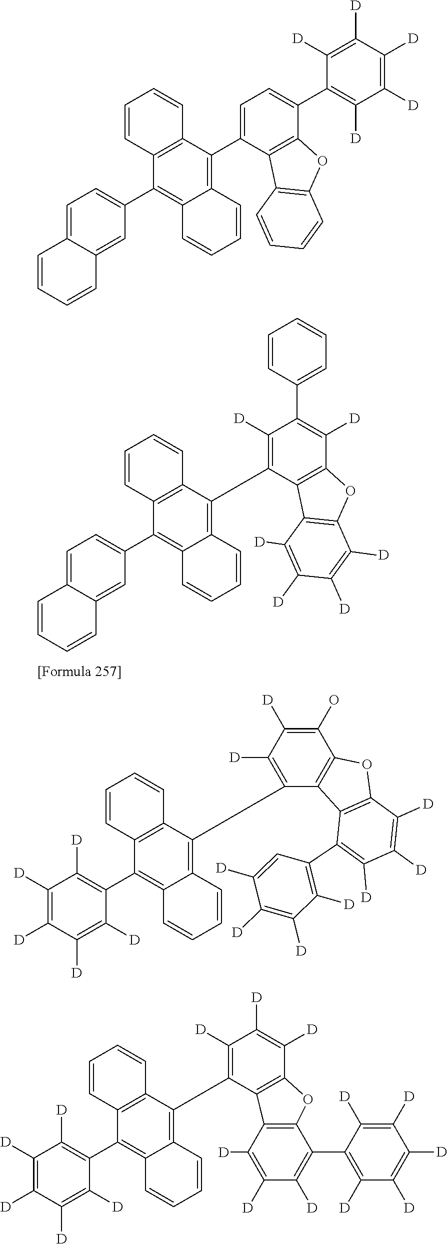

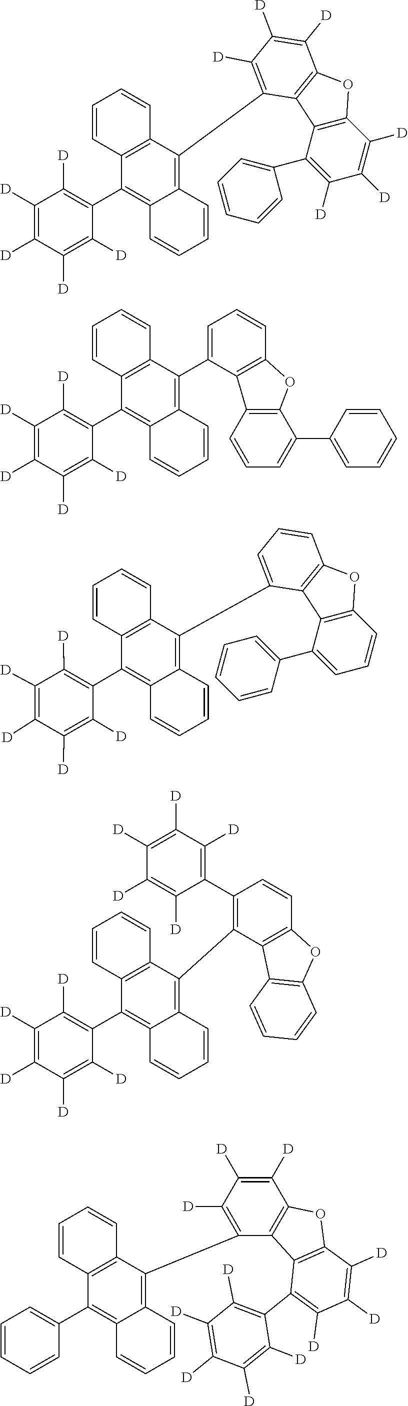

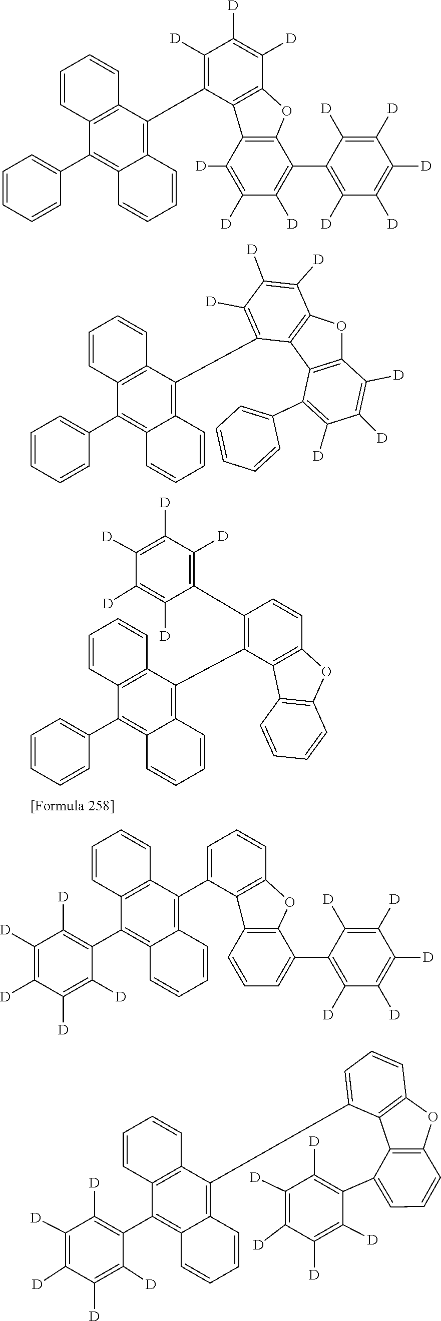

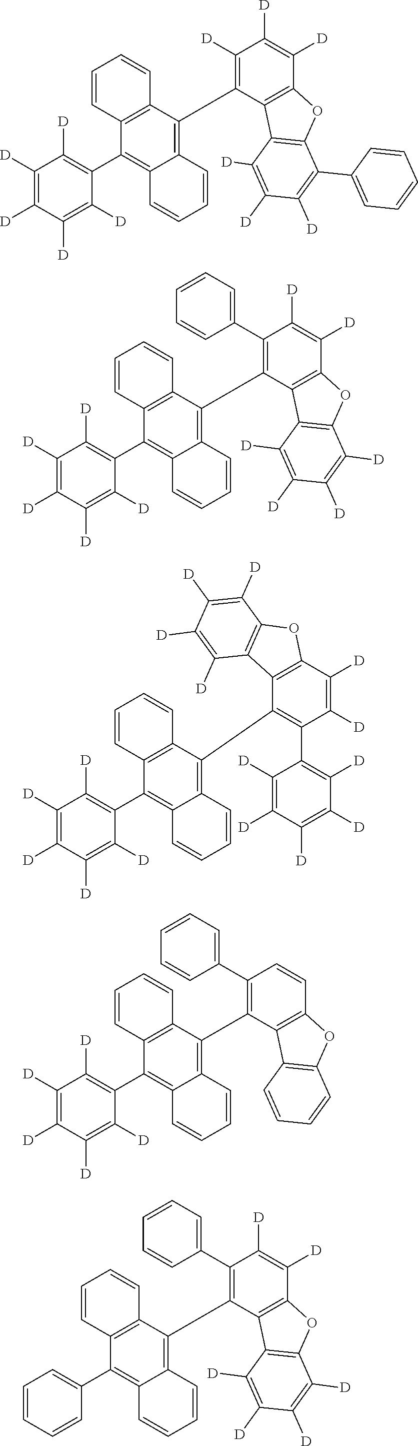

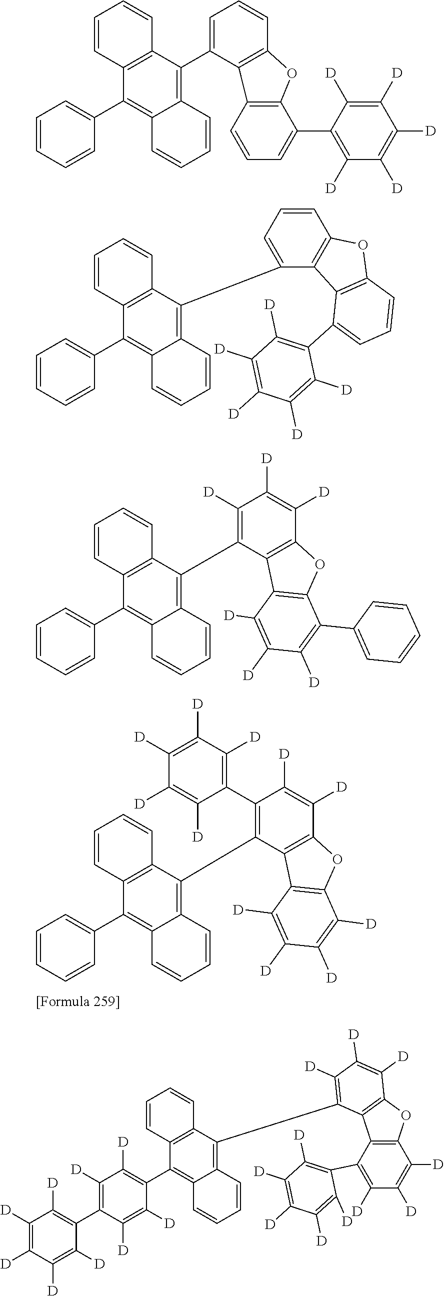

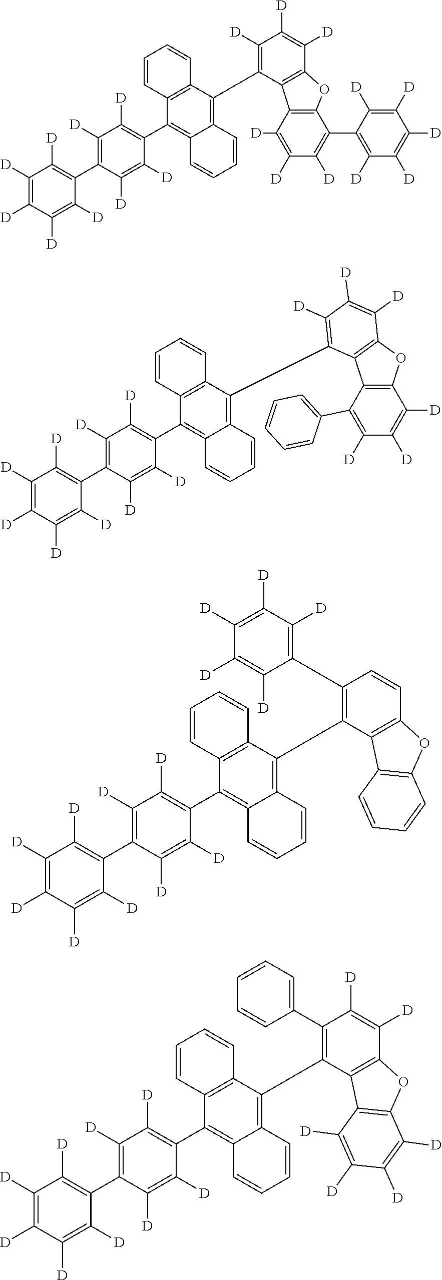

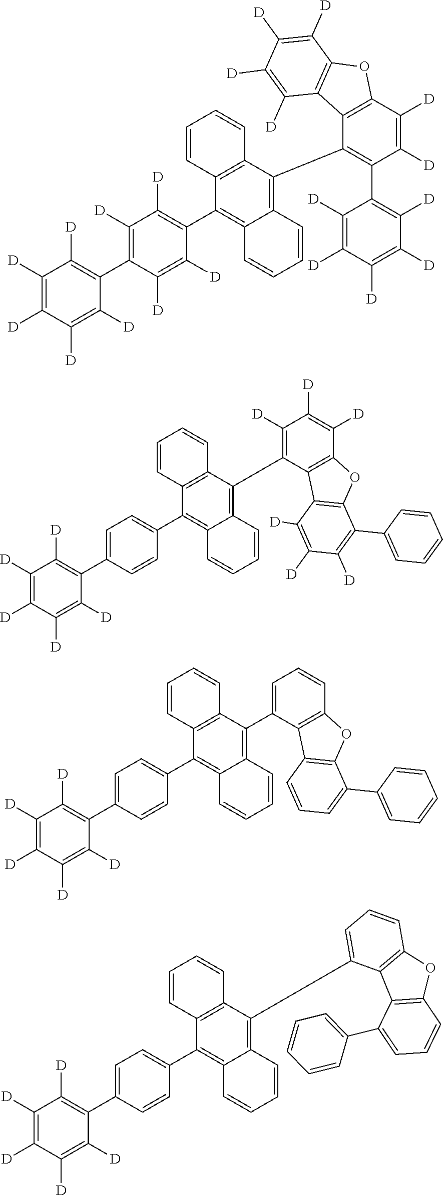

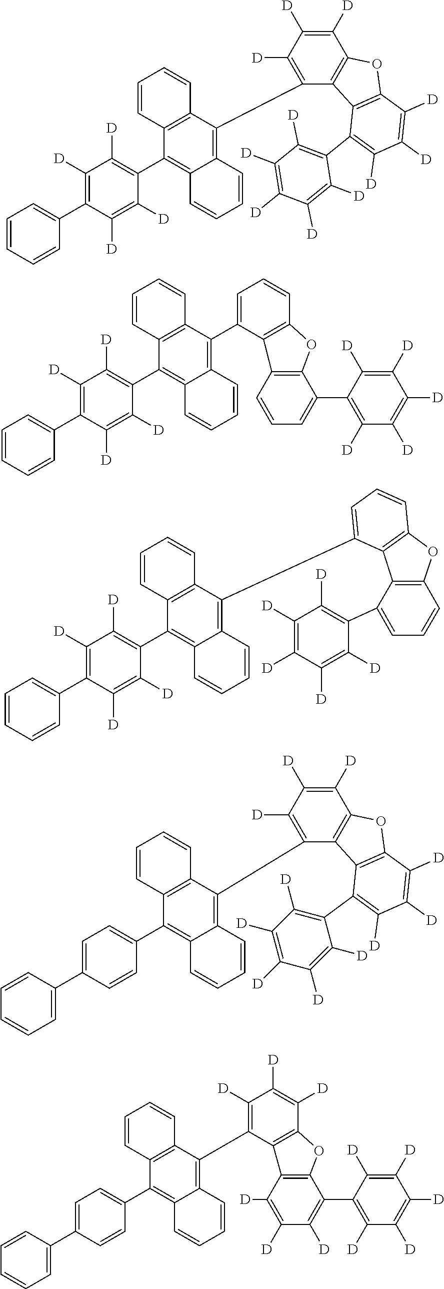

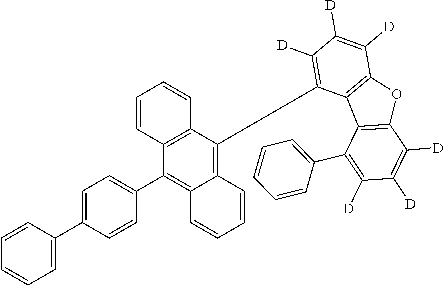 [SMILES: [2H]c1c([2H])c(-c2ccccc2)c2c(oc3c([2H])c([2H])c([2H])c(-c4c5ccccc5c(-c5ccc(-c6ccccc6)cc5)c5ccccc45)c32)c1[2H]]

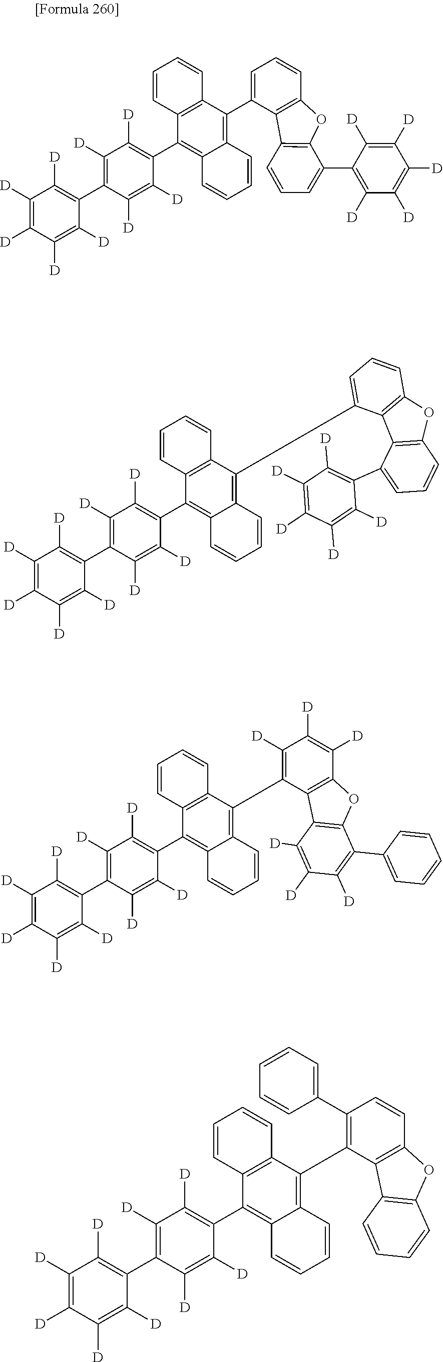

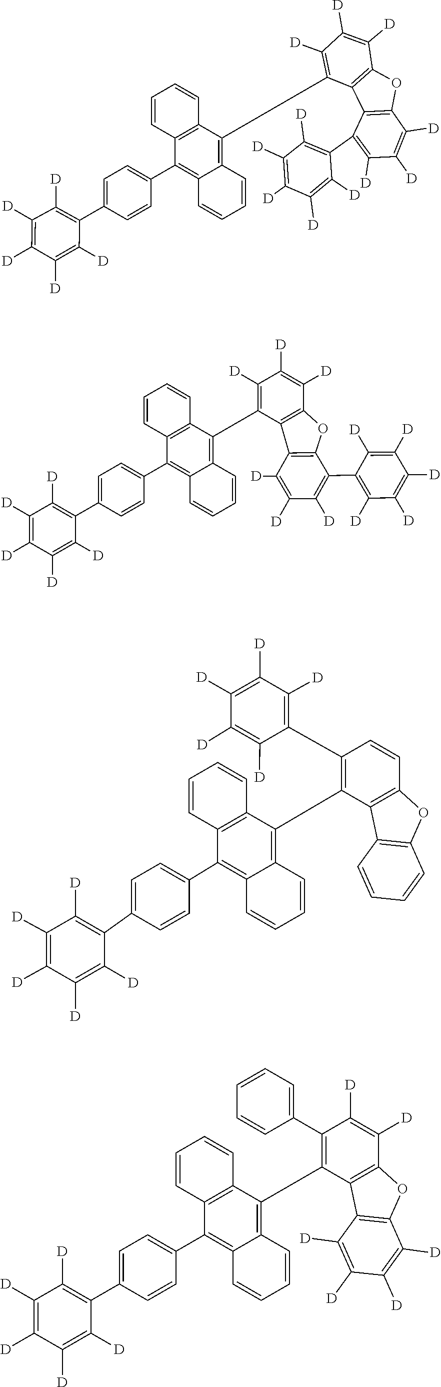

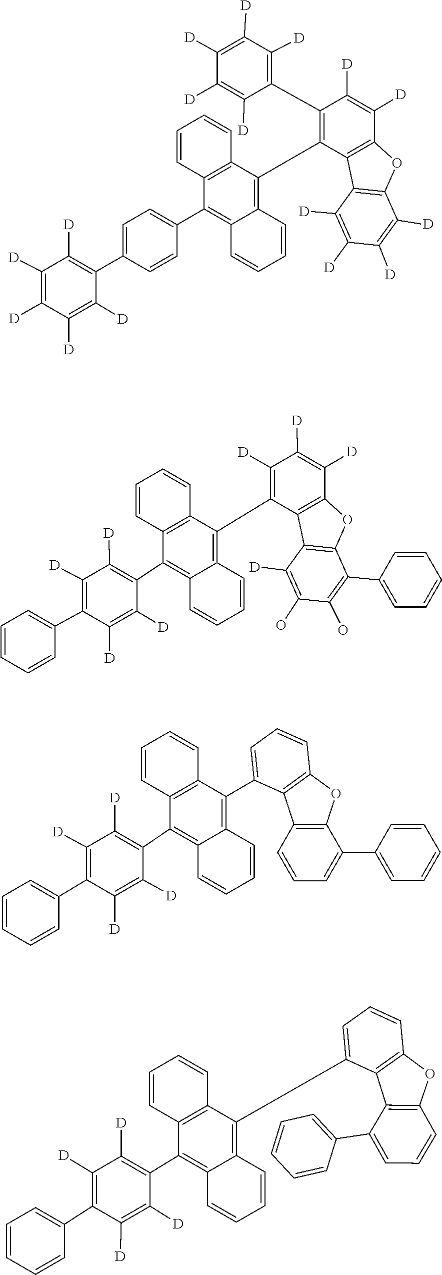

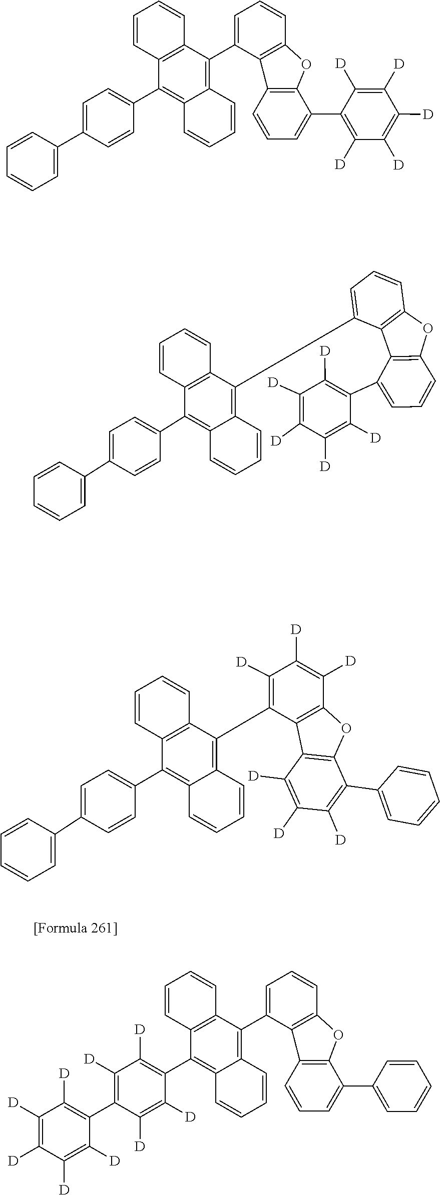 [SMILES: [2H]c1c([2H])c([2H])c(-c2cccc3c2oc2cccc(-c4c5ccccc5c(-c5ccc(-c6ccccc6)cc5)c5ccccc45)c23)c([2H])c1[2H] ; [2H]c1c([2H])c([2H])c(-c2cccc3oc4cccc(-c5c6ccccc6c(-c6ccc(-c7ccccc7)cc6)c6ccccc56)c4c23)c([2H])c1[2H] ; [2H]c1c([2H])c(-c2c3ccccc3c(-c3ccc(-c4ccccc4)cc3)c3ccccc23)c2c(oc3c(-c4ccccc4)c([2H])c([2H])c([2H])c32)c1[2H] ; [2H]c1c([2H])c([2H])c(-c2c([2H])c([2H])c(-c3c4ccccc4c(-c4cccc5oc6c(-c7ccccc7)cccc6c45)c4ccccc34)c([2H])c2[2H])c([2H])c1[2H]]

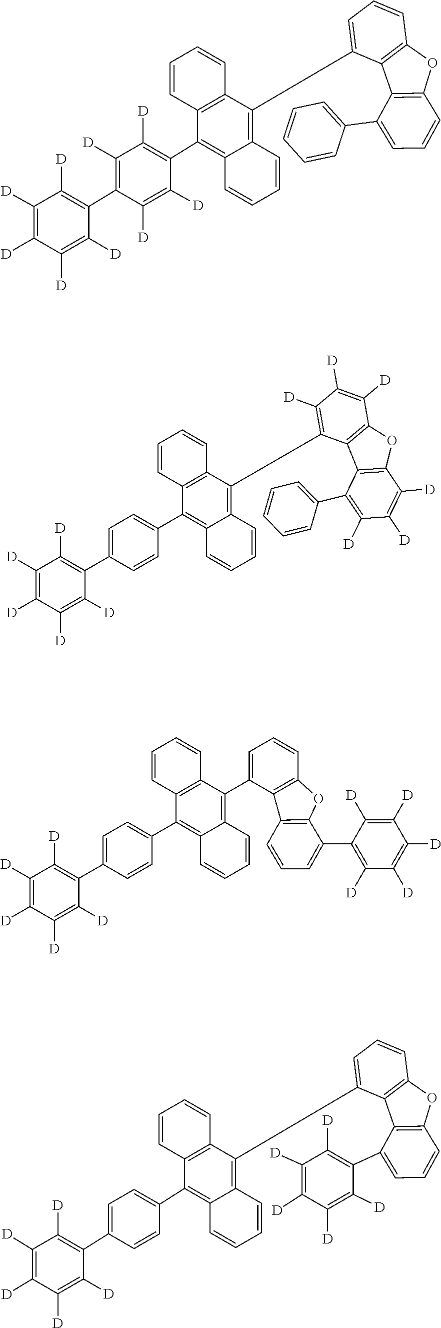

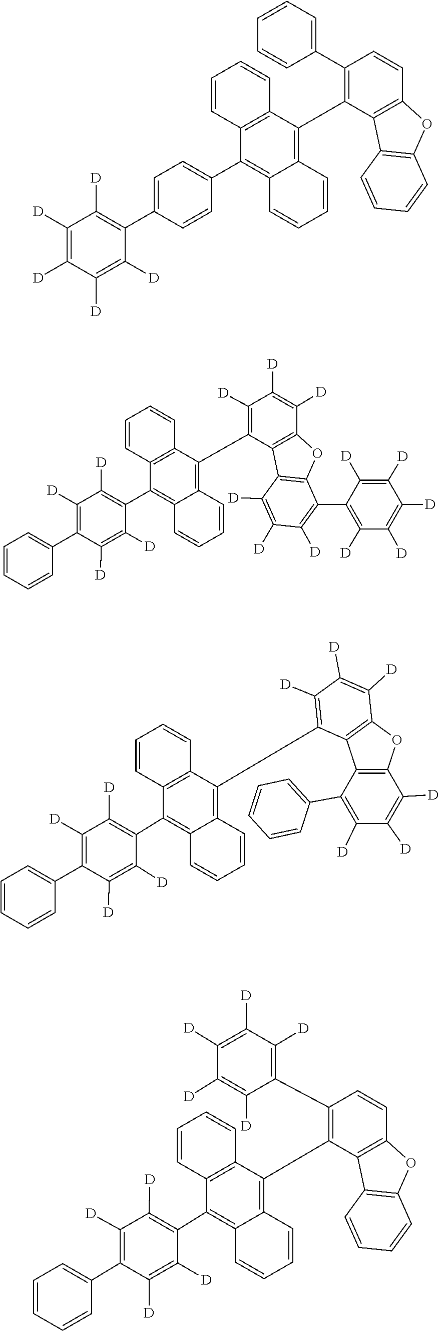

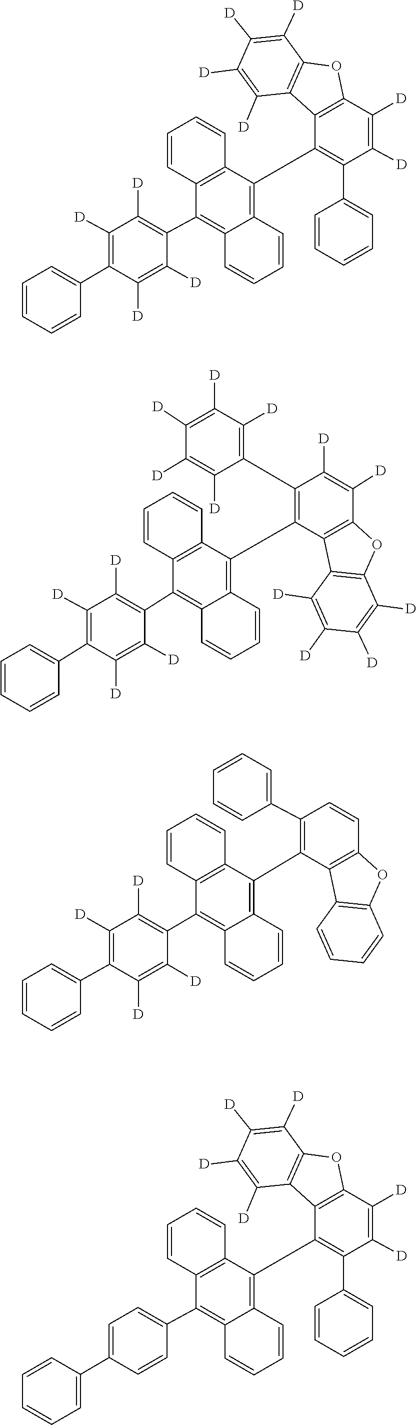

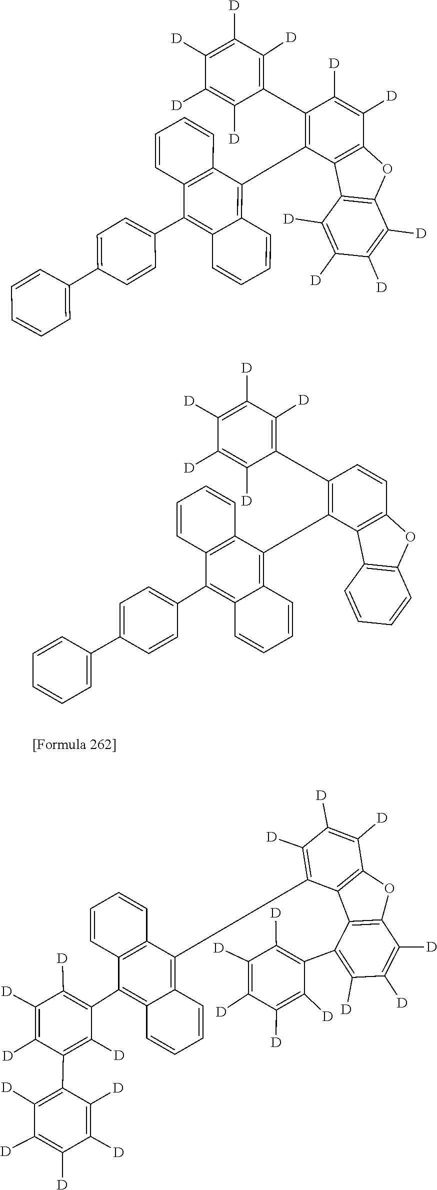 [SMILES: [2H]c1c([2H])c([2H])c(-c2c([2H])c([2H])c3oc4c([2H])c([2H])c([2H])c([2H])c4c3c2-c2c3ccccc3c(-c3ccc(-c4ccccc4)cc3)c3ccccc23)c([2H])c1[2H] ; [2H]c1c([2H])c([2H])c(-c2ccc3oc4ccccc4c3c2-c2c3ccccc3c(-c3ccc(-c4ccccc4)cc3)c3ccccc23)c([2H])c1[2H] ; [2H]c1c([2H])c([2H])c(-c2c([2H])c([2H])c([2H])c(-c3c4ccccc4c(-c4c([2H])c([2H])c([2H])c5oc6c([2H])c([2H])c([2H])c(-c7c([2H])c([2H])c([2H])c([2H])c7[2H])c6c45)c4ccccc34)c2[2H])c([2H])c1[2H]]

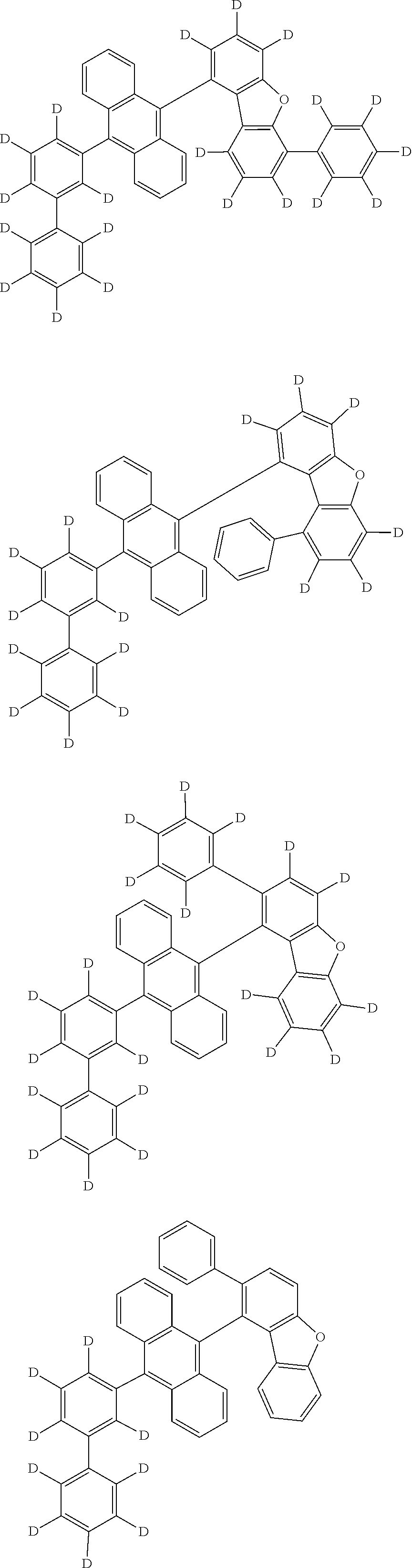

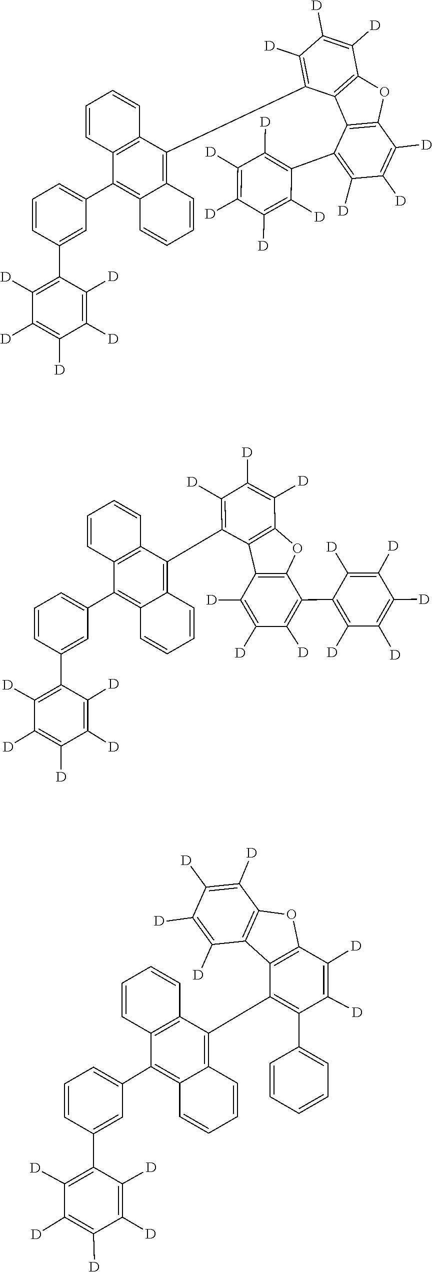 [SMILES: [2H]c1c([2H])c([2H])c(-c2cccc(-c3c4ccccc4c(-c4c([2H])c([2H])c([2H])c5oc6c([2H])c([2H])c([2H])c(-c7c([2H])c([2H])c([2H])c([2H])c7[2H])c6c45)c4ccccc34)c2)c([2H])c1[2H] ; [2H]c1c([2H])c([2H])c(-c2cccc(-c3c4ccccc4c(-c4c([2H])c([2H])c([2H])c5oc6c(-c7c([2H])c([2H])c([2H])c([2H])c7[2H])c([2H])c([2H])c([2H])c6c45)c4ccccc34)c2)c([2H])c1[2H] ; [2H]c1c([2H])c([2H])c(-c2cccc(-c3c4ccccc4c(-c4c(-c5ccccc5)c([2H])c([2H])c5oc6c([2H])c([2H])c([2H])c([2H])c6c45)c4ccccc34)c2)c([2H])c1[2H]]

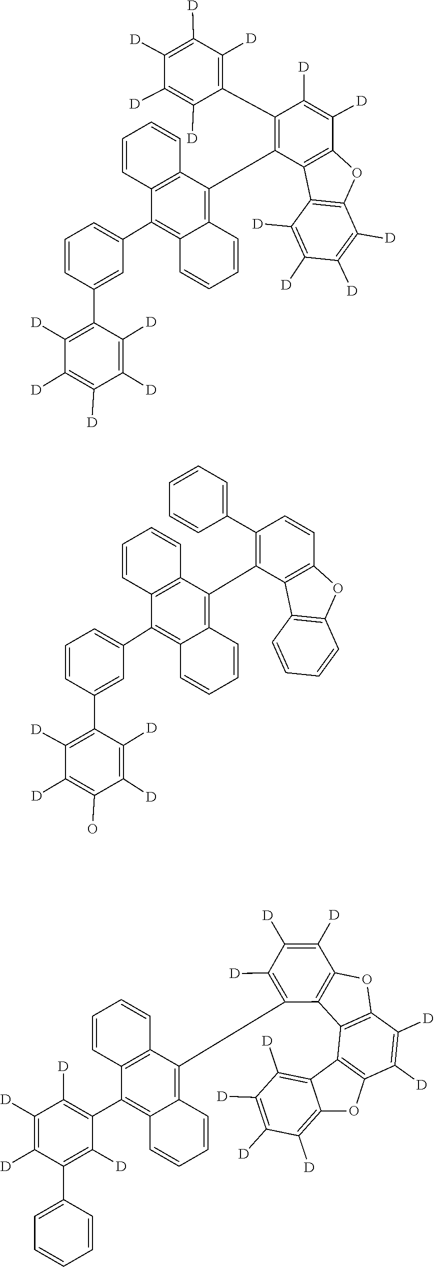

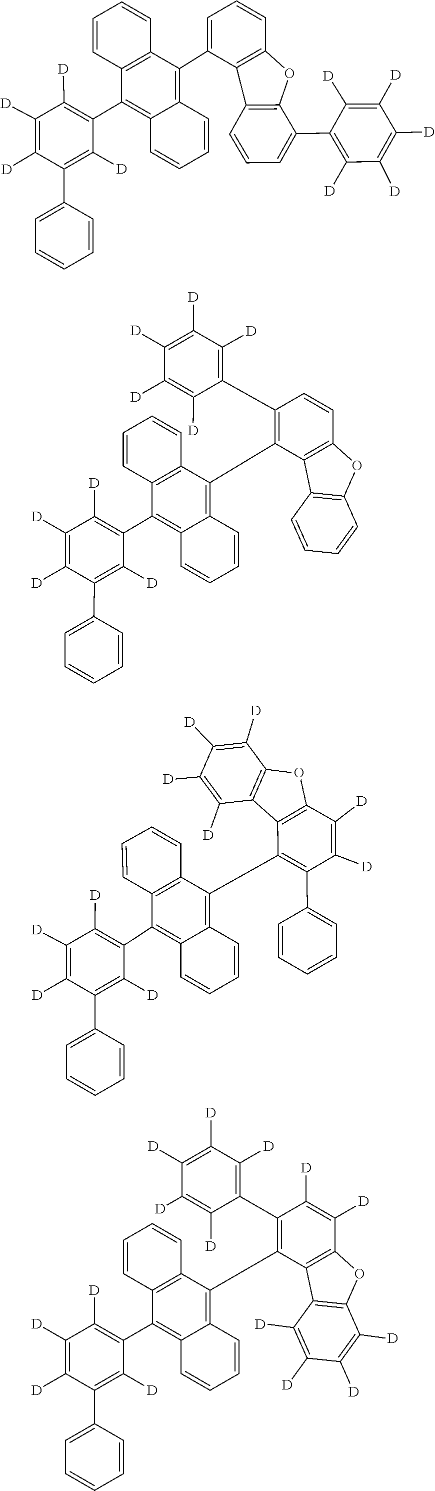

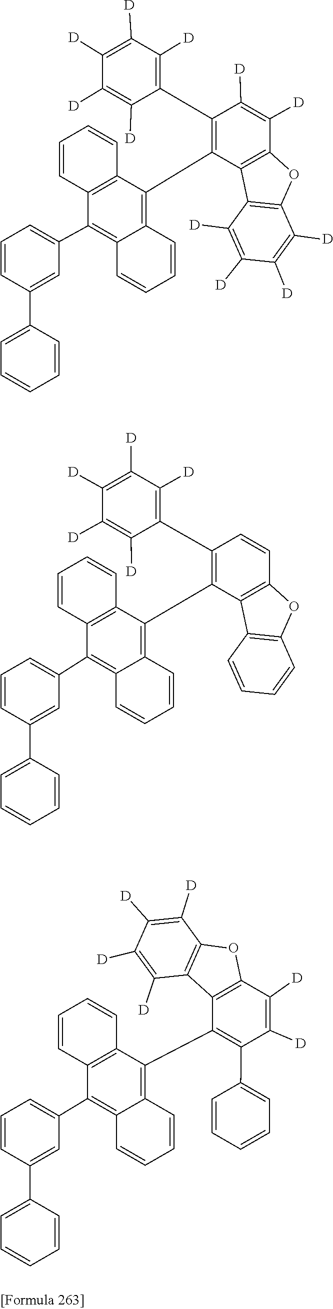 [SMILES: [2H]c1c([2H])c([2H])c(-c2c([2H])c([2H])c3oc4c([2H])c([2H])c([2H])c([2H])c4c3c2-c2c3ccccc3c(-c3cccc(-c4ccccc4)c3)c3ccccc23)c([2H])c1[2H] ; [2H]c1c([2H])c([2H])c(-c2ccc3oc4ccccc4c3c2-c2c3ccccc3c(-c3cccc(-c4ccccc4)c3)c3ccccc23)c([2H])c1[2H] ; [2H]c1c([2H])c([2H])c2c(oc3c([2H])c([2H])c(-c4ccccc4)c(-c4c5ccccc5c(-c5cccc(-c6ccccc6)c5)c5ccccc45)c32)c1[2H]]

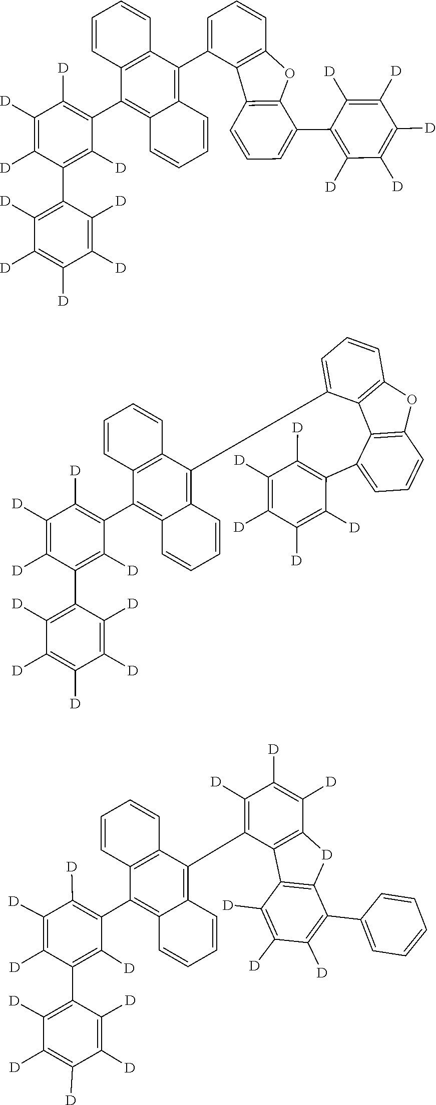

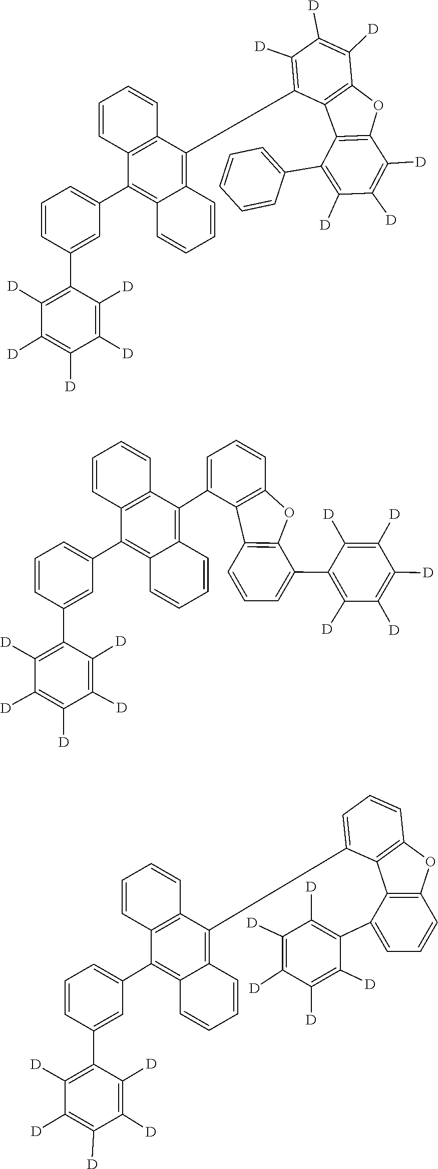 [SMILES: [2H]c1c([2H])c([2H])c(-c2cccc(-c3c4ccccc4c(-c4c([2H])c([2H])c([2H])c5oc6c([2H])c([2H])c([2H])c(-c7ccccc7)c6c45)c4ccccc34)c2)c([2H])c1[2H] ; [2H]c1c([2H])c([2H])c(-c2cccc(-c3c4ccccc4c(-c4cccc5oc6c(-c7c([2H])c([2H])c([2H])c([2H])c7[2H])cccc6c45)c4ccccc34)c2)c([2H])c1[2H] ; [2H]c1c([2H])c([2H])c(-c2cccc(-c3c4ccccc4c(-c4cccc5oc6cccc(-c7c([2H])c([2H])c([2H])c([2H])c7[2H])c6c45)c4ccccc34)c2)c([2H])c1[2H]]

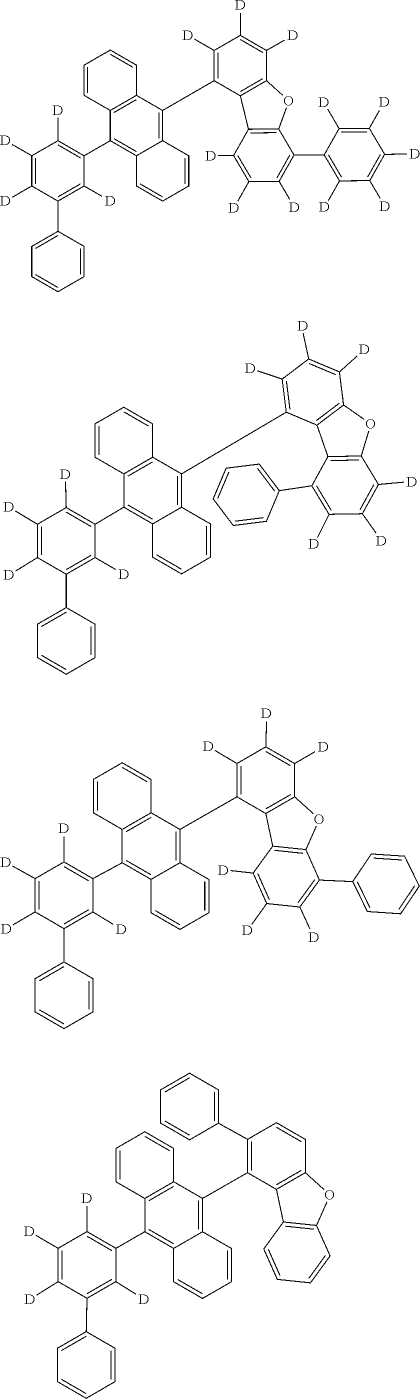

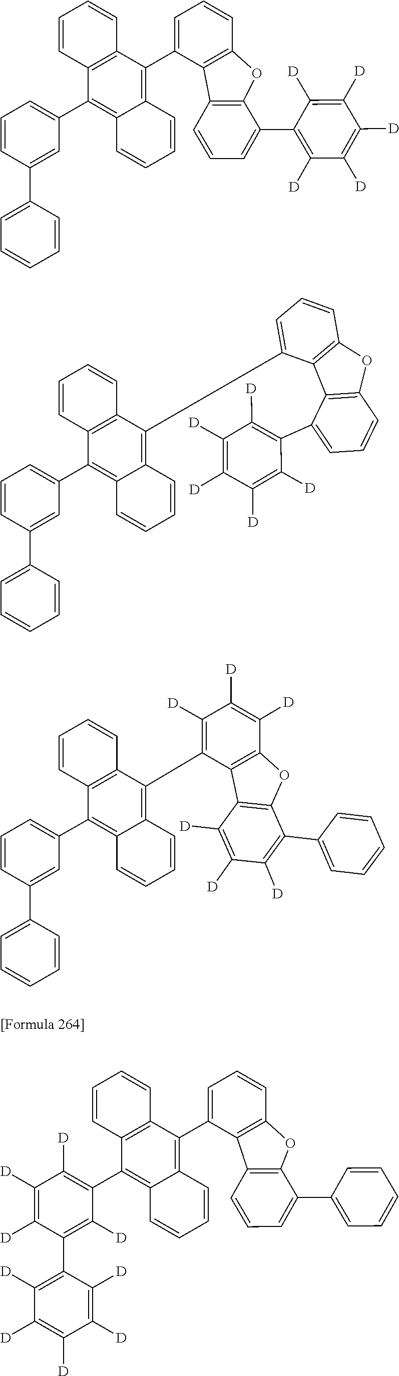 [SMILES: [2H]c1c([2H])c([2H])c(-c2cccc3c2oc2cccc(-c4c5ccccc5c(-c5cccc(-c6ccccc6)c5)c5ccccc45)c23)c([2H])c1[2H] ; [2H]c1c([2H])c([2H])c(-c2cccc3oc4cccc(-c5c6ccccc6c(-c6cccc(-c7ccccc7)c6)c6ccccc56)c4c23)c([2H])c1[2H] ; [2H]c1c([2H])c(-c2c3ccccc3c(-c3cccc(-c4ccccc4)c3)c3ccccc23)c2c(oc3c(-c4ccccc4)c([2H])c([2H])c([2H])c32)c1[2H] ; [2H]c1c([2H])c([2H])c(-c2c([2H])c([2H])c([2H])c(-c3c4ccccc4c(-c4cccc5oc6c(-c7ccccc7)cccc6c45)c4ccccc34)c2[2H])c([2H])c1[2H]]

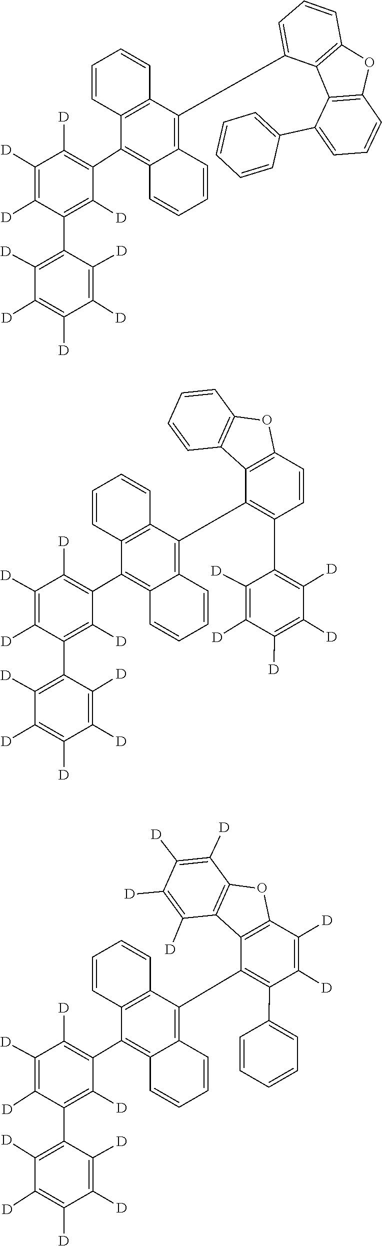 [SMILES: [2H]c1c([2H])c([2H])c(-c2c([2H])c([2H])c([2H])c(-c3c4ccccc4c(-c4cccc5oc6cccc(-c7ccccc7)c6c45)c4ccccc34)c2[2H])c([2H])c1[2H] ; [2H]c1c([2H])c([2H])c(-c2ccc3oc4ccccc4c3c2-c2c3ccccc3c(-c3c([2H])c([2H])c([2H])c(-c4c([2H])c([2H])c([2H])c([2H])c4[2H])c3[2H])c3ccccc23)c([2H])c1[2H] ; [2H]c1c([2H])c([2H])c(-c2c([2H])c([2H])c([2H])c(-c3c4ccccc4c(-c4c(-c5ccccc5)c([2H])c([2H])c5oc6c([2H])c([2H])c([2H])c([2H])c6c45)c4ccccc34)c2[2H])c([2H])c1[2H]]

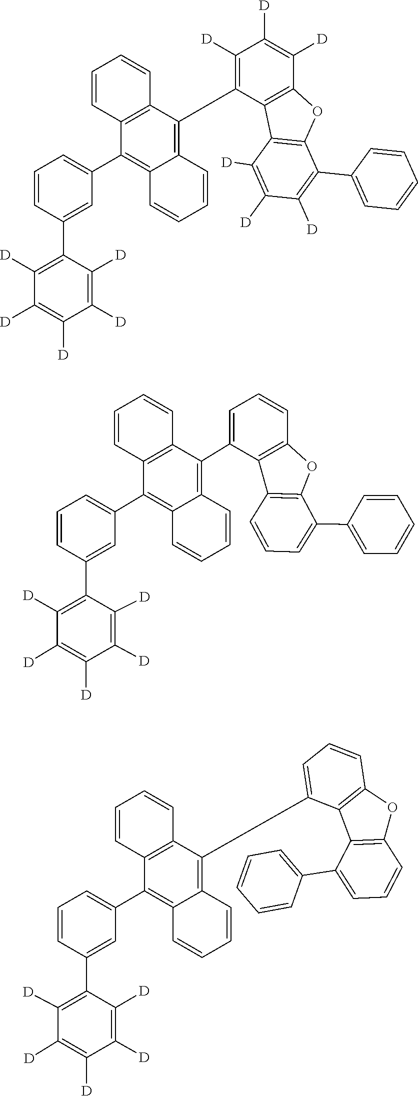 [SMILES: [2H]c1c([2H])c([2H])c(-c2cccc(-c3c4ccccc4c(-c4c([2H])c([2H])c([2H])c5oc6c(-c7ccccc7)c([2H])c([2H])c([2H])c6c45)c4ccccc34)c2)c([2H])c1[2H] ; [2H]c1c([2H])c([2H])c(-c2cccc(-c3c4ccccc4c(-c4cccc5oc6c(-c7ccccc7)cccc6c45)c4ccccc34)c2)c([2H])c1[2H] ; [2H]c1c([2H])c([2H])c(-c2cccc(-c3c4ccccc4c(-c4cccc5oc6cccc(-c7ccccc7)c6c45)c4ccccc34)c2)c([2H])c1[2H]]

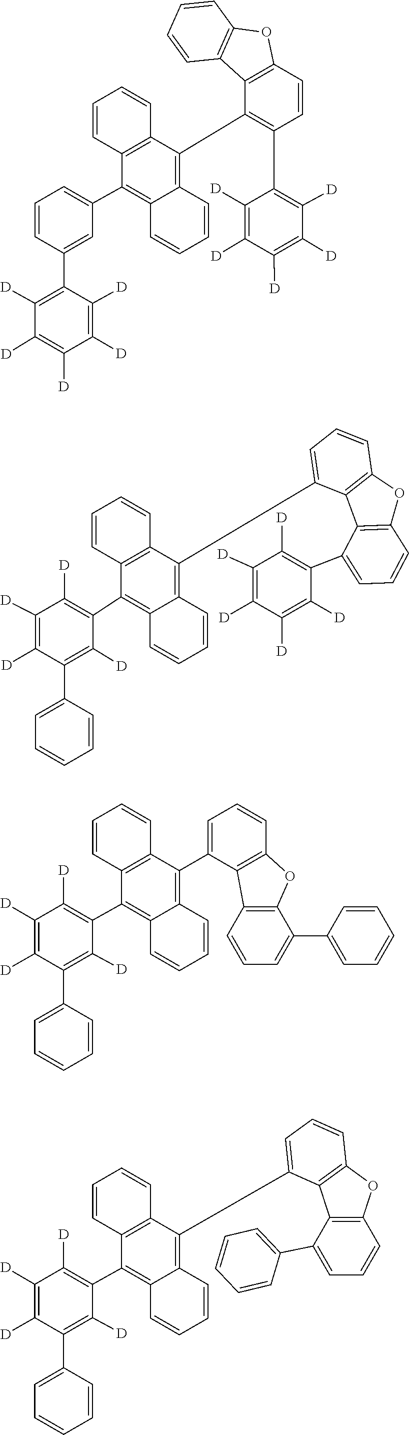 [SMILES: [2H]c1c([2H])c([2H])c(-c2cccc(-c3c4ccccc4c(-c4c(-c5c([2H])c([2H])c([2H])c([2H])c5[2H])ccc5oc6ccccc6c45)c4ccccc34)c2)c([2H])c1[2H] ; [2H]c1c([2H])c([2H])c(-c2cccc3oc4cccc(-c5c6ccccc6c(-c6c([2H])c([2H])c([2H])c(-c7ccccc7)c6[2H])c6ccccc56)c4c23)c([2H])c1[2H] ; [2H]c1c([2H])c(-c2ccccc2)c([2H])c(-c2c3ccccc3c(-c3cccc4oc5c(-c6ccccc6)cccc5c34)c3ccccc23)c1[2H] ; [2H]c1c([2H])c(-c2ccccc2)c([2H])c(-c2c3ccccc3c(-c3cccc4oc5cccc(-c6ccccc6)c5c34)c3ccccc23)c1[2H]]

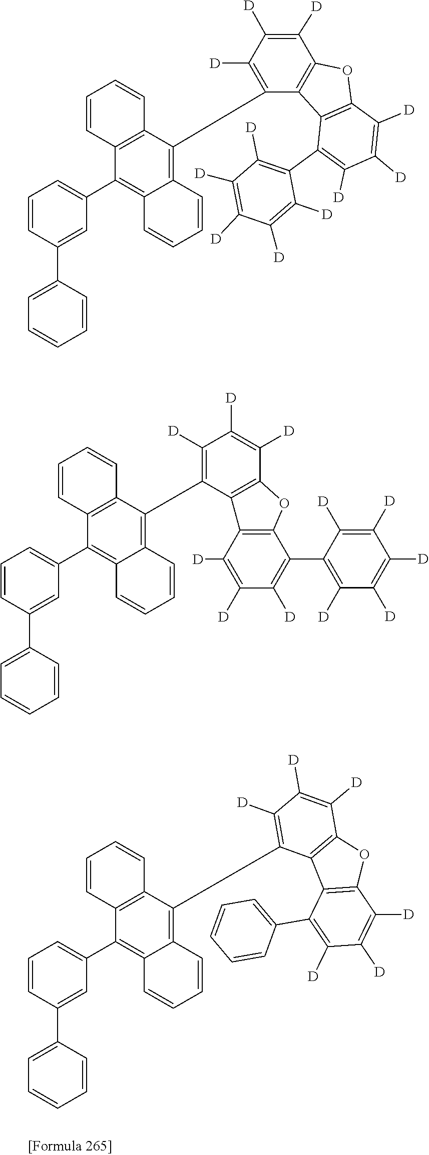 [SMILES: [2H]c1c([2H])c([2H])c(-c2c([2H])c([2H])c([2H])c3oc4c([2H])c([2H])c([2H])c(-c5c6ccccc6c(-c6cccc(-c7ccccc7)c6)c6ccccc56)c4c23)c([2H])c1[2H] ; [2H]c1c([2H])c([2H])c(-c2c([2H])c([2H])c([2H])c3c2oc2c([2H])c([2H])c([2H])c(-c4c5ccccc5c(-c5cccc(-c6ccccc6)c5)c5ccccc45)c23)c([2H])c1[2H] ; [2H]c1c([2H])c(-c2ccccc2)c2c(oc3c([2H])c([2H])c([2H])c(-c4c5ccccc5c(-c5cccc(-c6ccccc6)c5)c5ccccc45)c32)c1[2H]]

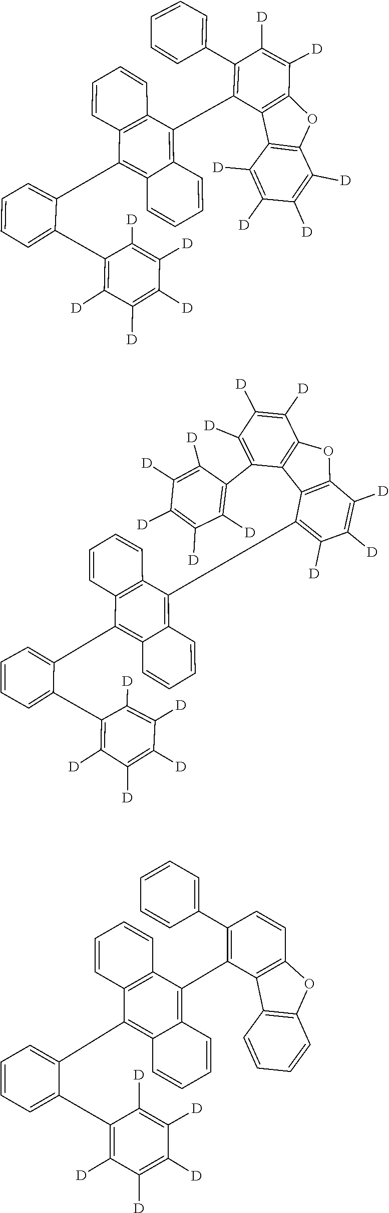 [SMILES: [2H]c1c([2H])c([2H])c(-c2ccccc2-c2c3ccccc3c(-c3c(-c4ccccc4)c([2H])c([2H])c4oc5c([2H])c([2H])c([2H])c([2H])c5c34)c3ccccc23)c([2H])c1[2H] ; [2H]c1c([2H])c([2H])c(-c2ccccc2-c2c3ccccc3c(-c3c([2H])c([2H])c([2H])c4oc5c([2H])c([2H])c([2H])c(-c6c([2H])c([2H])c([2H])c([2H])c6[2H])c5c34)c3ccccc23)c([2H])c1[2H] ; [2H]c1c([2H])c([2H])c(-c2ccccc2-c2c3ccccc3c(-c3c(-c4ccccc4)ccc4oc5ccccc5c34)c3ccccc23)c([2H])c1[2H]]

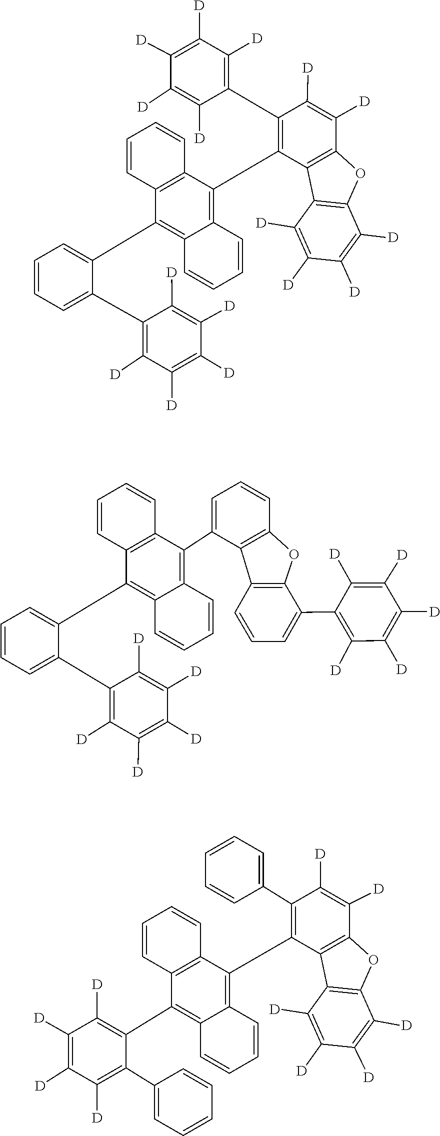 [SMILES: [2H]c1c([2H])c([2H])c(-c2ccccc2-c2c3ccccc3c(-c3c(-c4c([2H])c([2H])c([2H])c([2H])c4[2H])c([2H])c([2H])c4oc5c([2H])c([2H])c([2H])c([2H])c5c34)c3ccccc23)c([2H])c1[2H] ; [2H]c1c([2H])c([2H])c(-c2ccccc2-c2c3ccccc3c(-c3cccc4oc5c(-c6c([2H])c([2H])c([2H])c([2H])c6[2H])cccc5c34)c3ccccc23)c([2H])c1[2H] ; [2H]c1c([2H])c([2H])c(-c2c3ccccc3c(-c3c(-c4ccccc4)c([2H])c([2H])c4oc5c([2H])c([2H])c([2H])c([2H])c5c34)c3ccccc23)c(-c2ccccc2)c1[2H]]

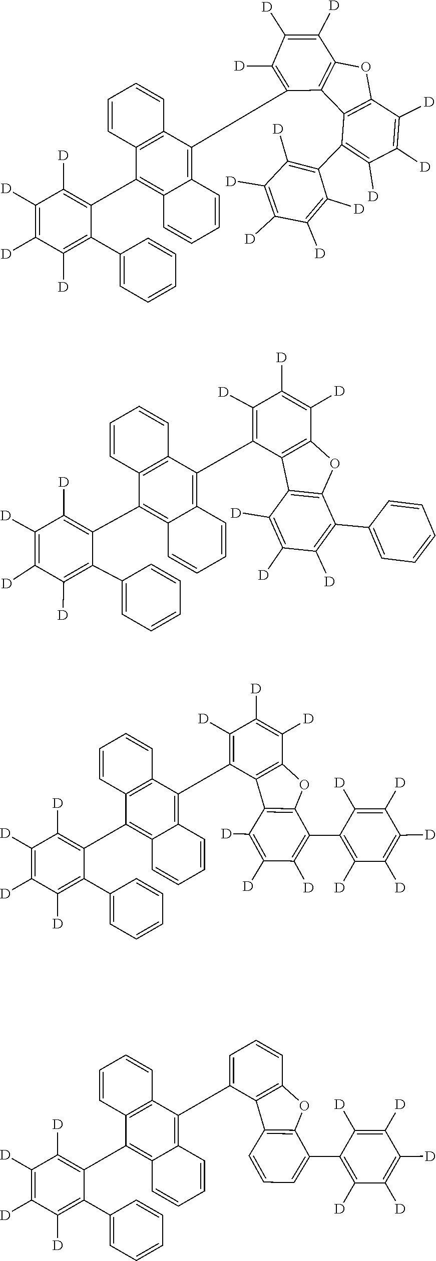

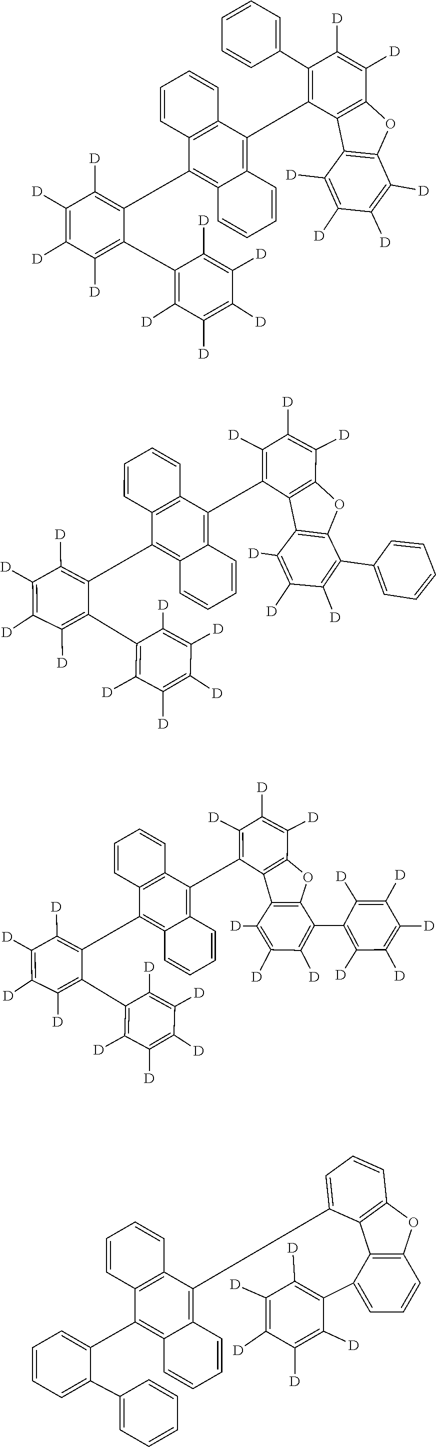

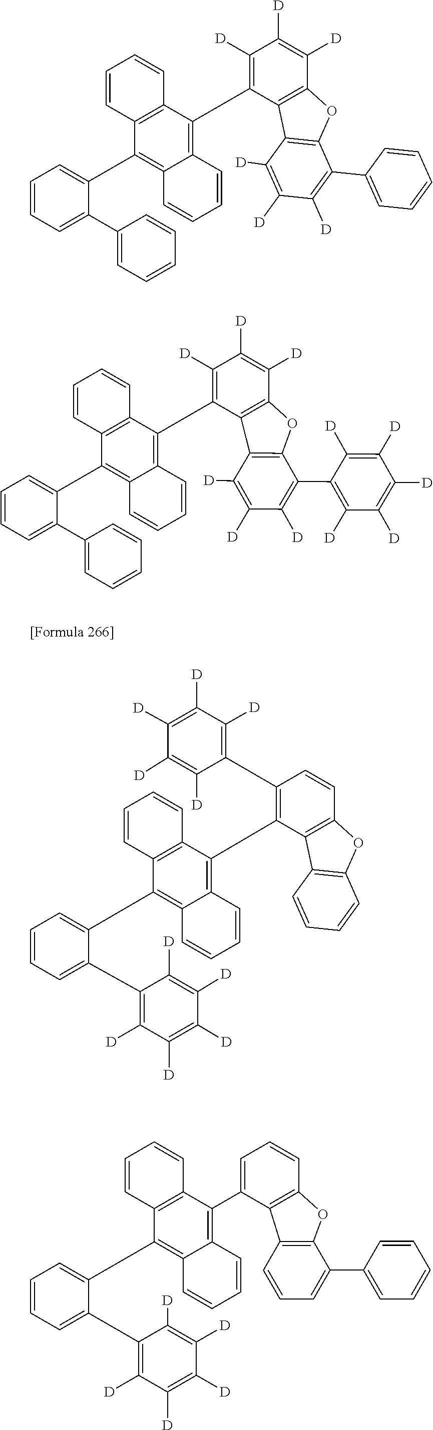 [SMILES: [2H]c1c([2H])c(-c2c3ccccc3c(-c3ccccc3-c3ccccc3)c3ccccc23)c2c(oc3c(-c4ccccc4)c([2H])c([2H])c([2H])c32)c1[2H] ; [2H]c1c([2H])c([2H])c(-c2c([2H])c([2H])c([2H])c3c2oc2c([2H])c([2H])c([2H])c(-c4c5ccccc5c(-c5ccccc5-c5ccccc5)c5ccccc45)c23)c([2H])c1[2H] ; [2H]c1c([2H])c([2H])c(-c2ccccc2-c2c3ccccc3c(-c3c(-c4c([2H])c([2H])c([2H])c([2H])c4[2H])ccc4oc5ccccc5c34)c3ccccc23)c([2H])c1[2H] ; [2H]c1c([2H])c([2H])c(-c2ccccc2-c2c3ccccc3c(-c3cccc4oc5c(-c6ccccc6)cccc5c34)c3ccccc23)c([2H])c1[2H]]

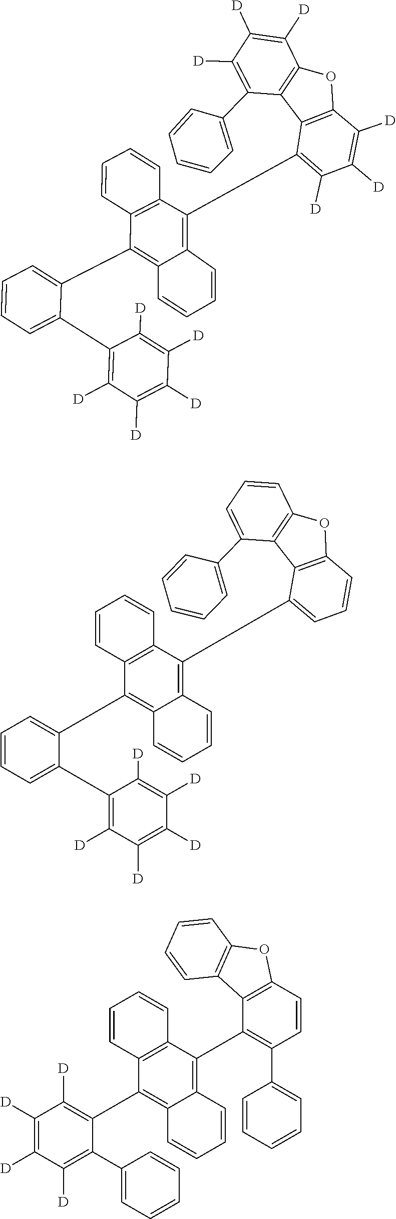 [SMILES: [2H]c1c([2H])c([2H])c(-c2ccccc2-c2c3ccccc3c(-c3c([2H])c([2H])c([2H])c4oc5c([2H])c([2H])c([2H])c(-c6ccccc6)c5c34)c3ccccc23)c([2H])c1[2H] ; [2H]c1c([2H])c([2H])c(-c2ccccc2-c2c3ccccc3c(-c3cccc4oc5cccc(-c6ccccc6)c5c34)c3ccccc23)c([2H])c1[2H] ; [2H]c1c([2H])c([2H])c(-c2c3ccccc3c(-c3c(-c4ccccc4)ccc4oc5ccccc5c34)c3ccccc23)c(-c2ccccc2)c1[2H]]

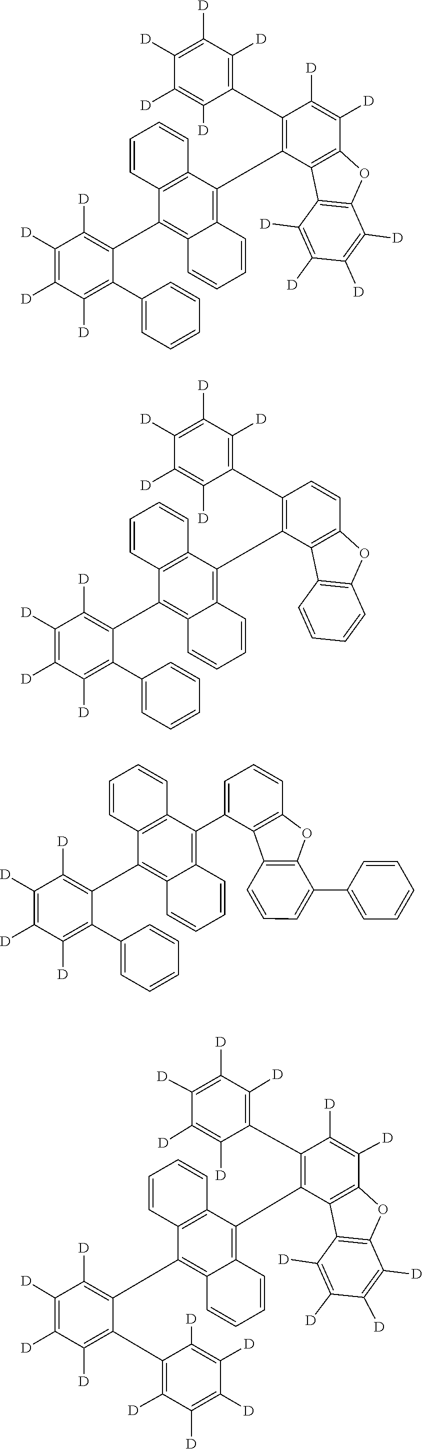

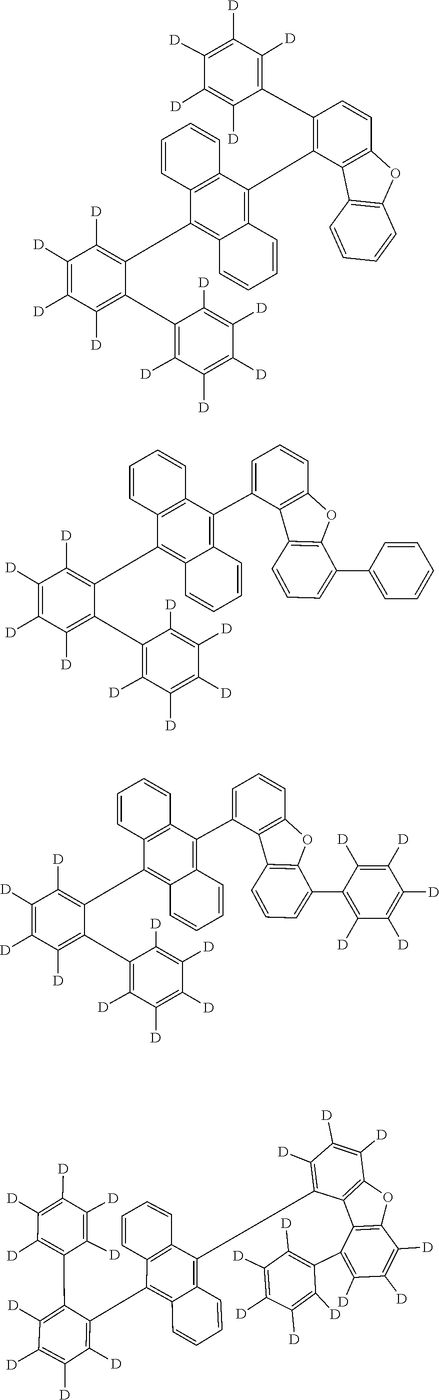

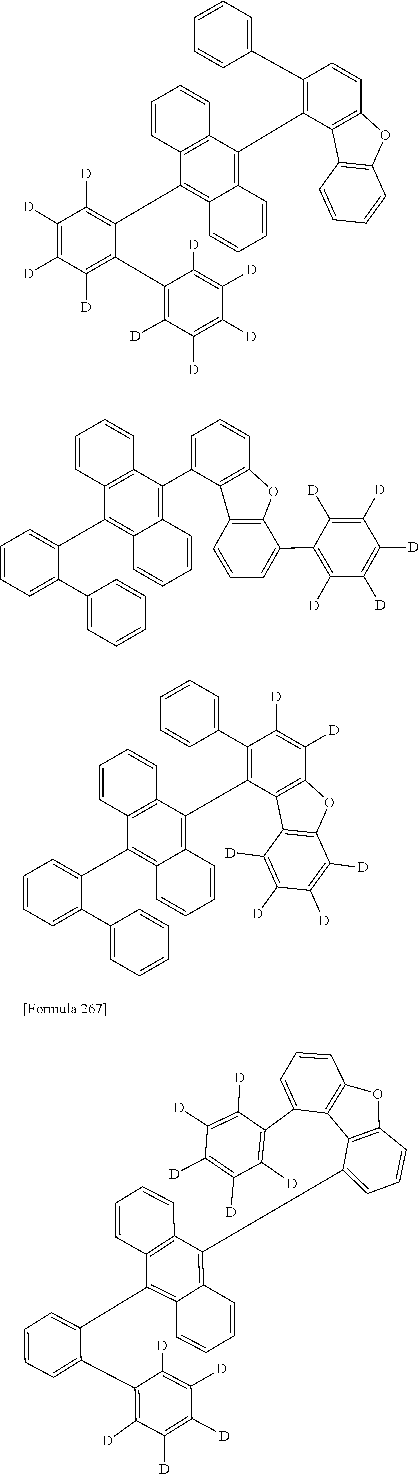 [SMILES: [2H]c1c([2H])c([2H])c(-c2c([2H])c([2H])c([2H])c([2H])c2-c2c3ccccc3c(-c3c(-c4ccccc4)ccc4oc5ccccc5c34)c3ccccc23)c([2H])c1[2H] ; [2H]c1c([2H])c([2H])c(-c2cccc3c2oc2cccc(-c4c5ccccc5c(-c5ccccc5-c5ccccc5)c5ccccc45)c23)c([2H])c1[2H] ; [2H]c1c([2H])c([2H])c2c(oc3c([2H])c([2H])c(-c4ccccc4)c(-c4c5ccccc5c(-c5ccccc5-c5ccccc5)c5ccccc45)c32)c1[2H] ; [2H]c1c([2H])c([2H])c(-c2ccccc2-c2c3ccccc3c(-c3cccc4oc5cccc(-c6c([2H])c([2H])c([2H])c([2H])c6[2H])c5c34)c3ccccc23)c([2H])c1[2H]]

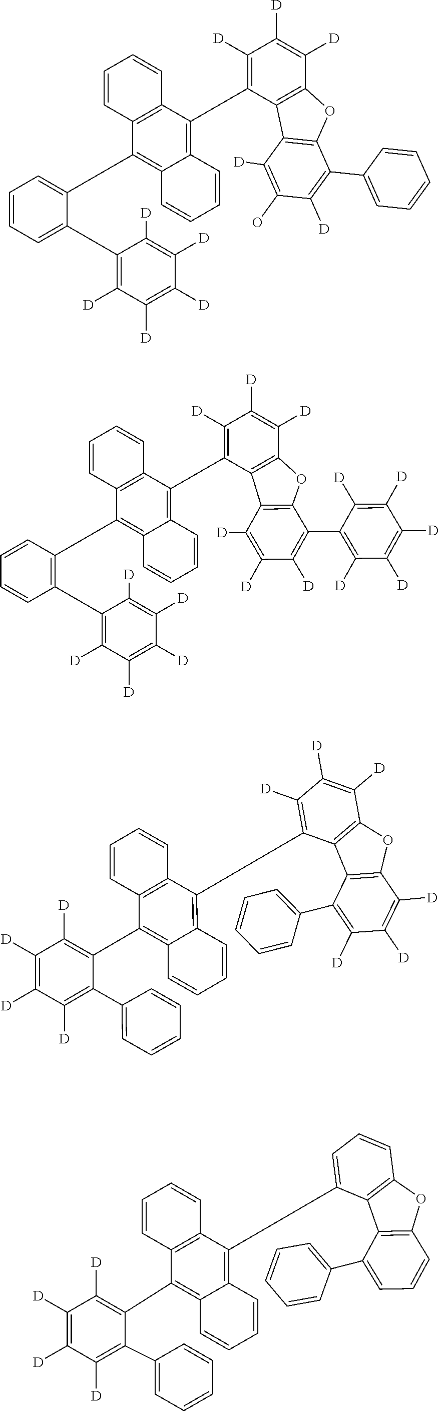

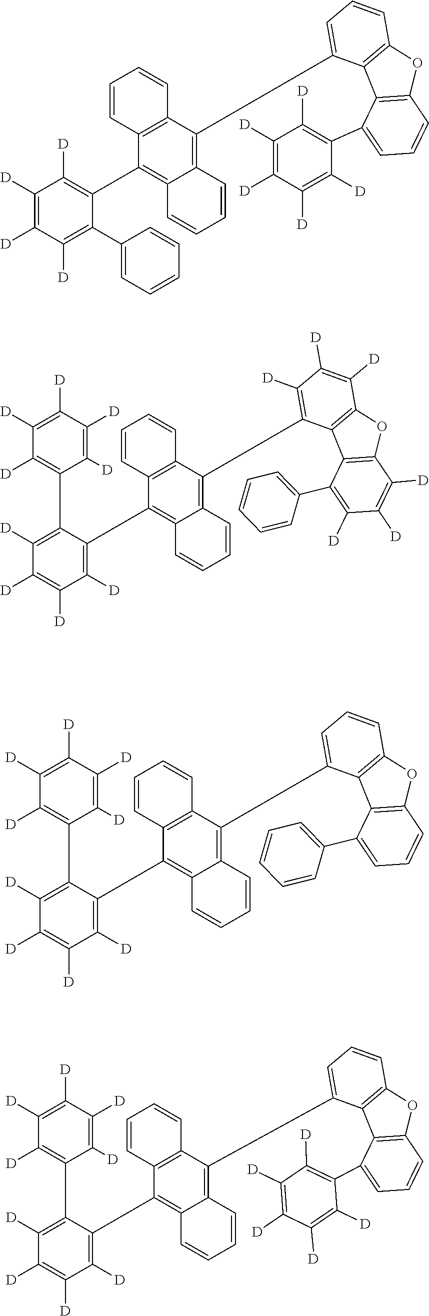

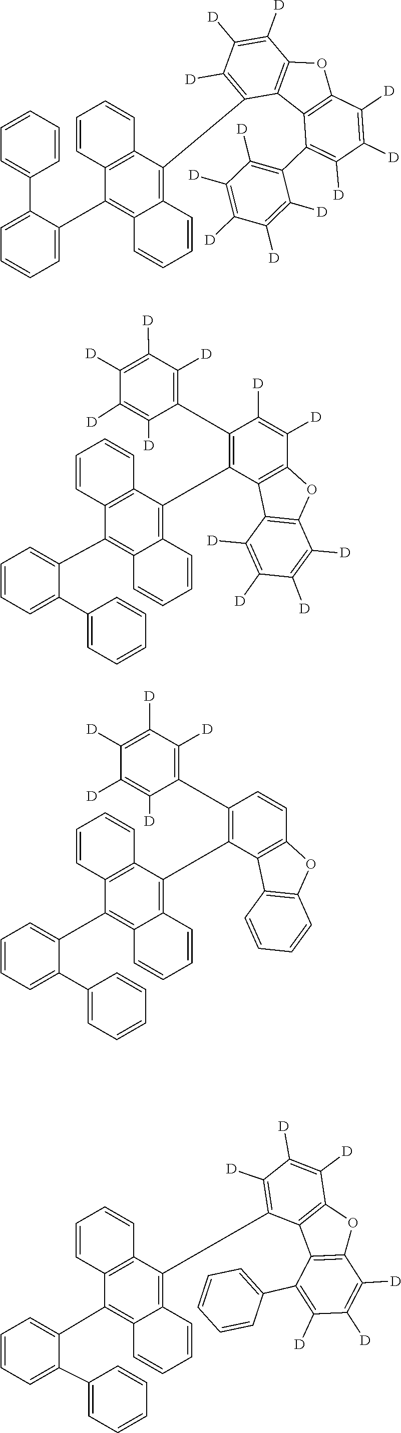

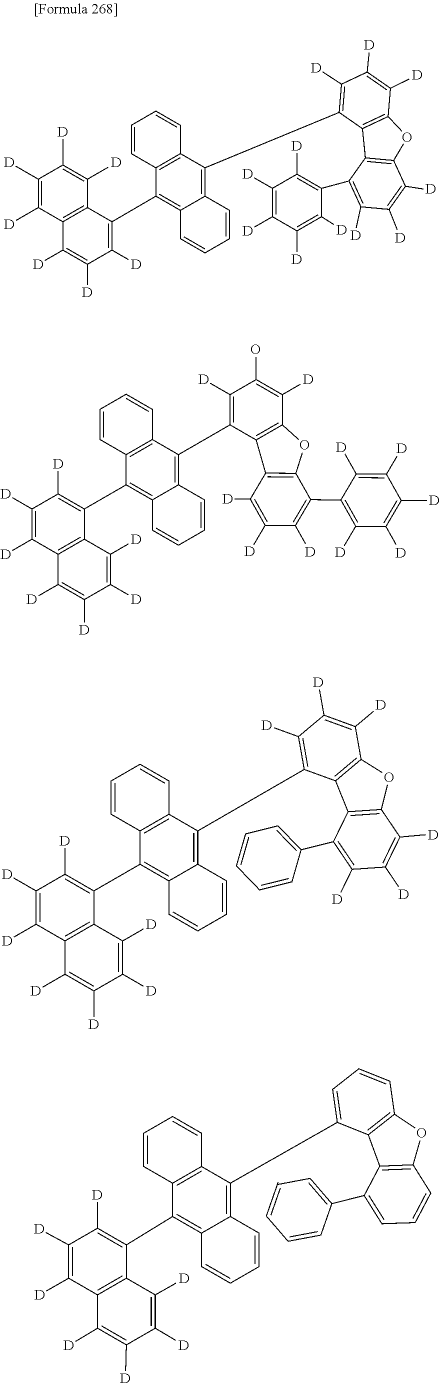

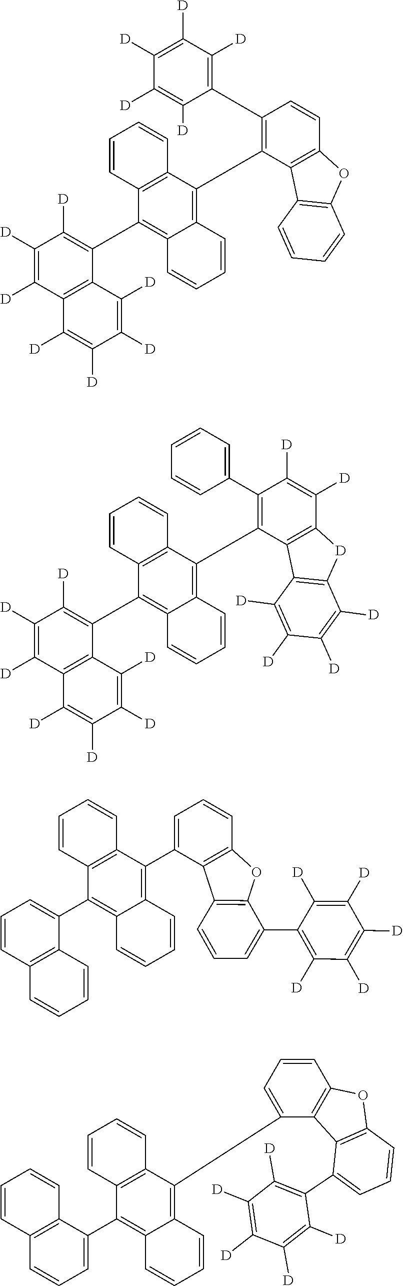

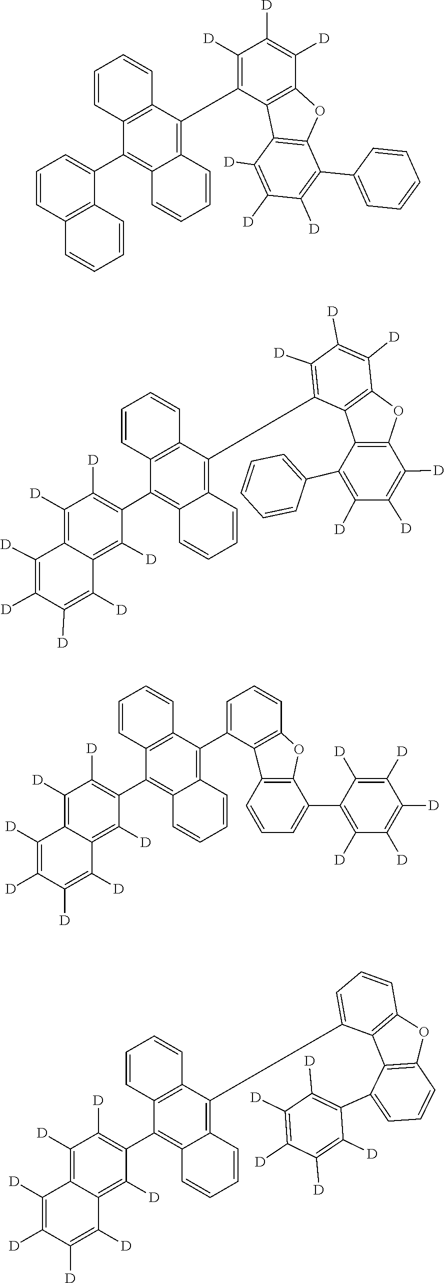

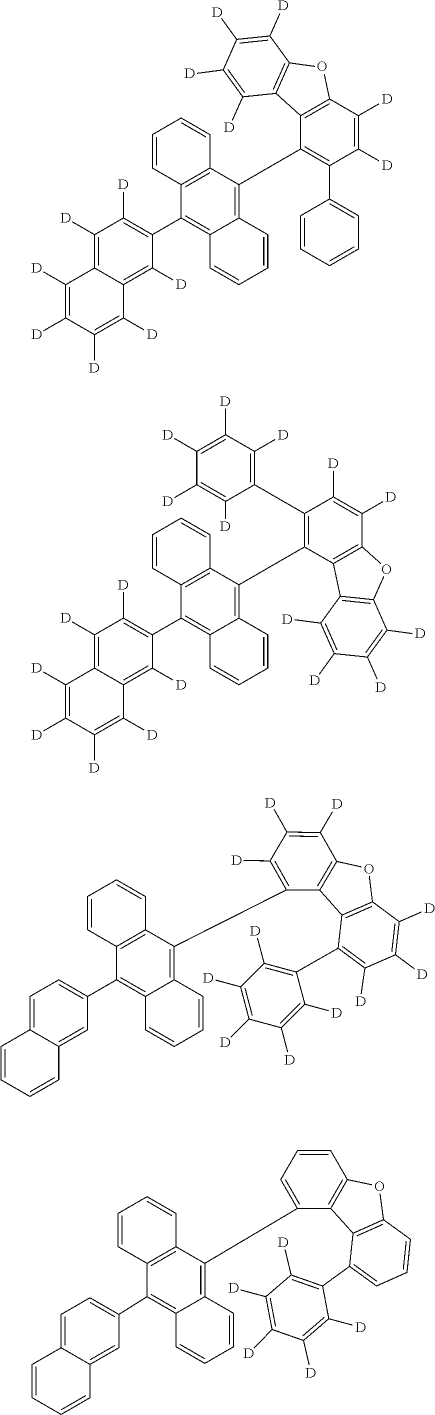

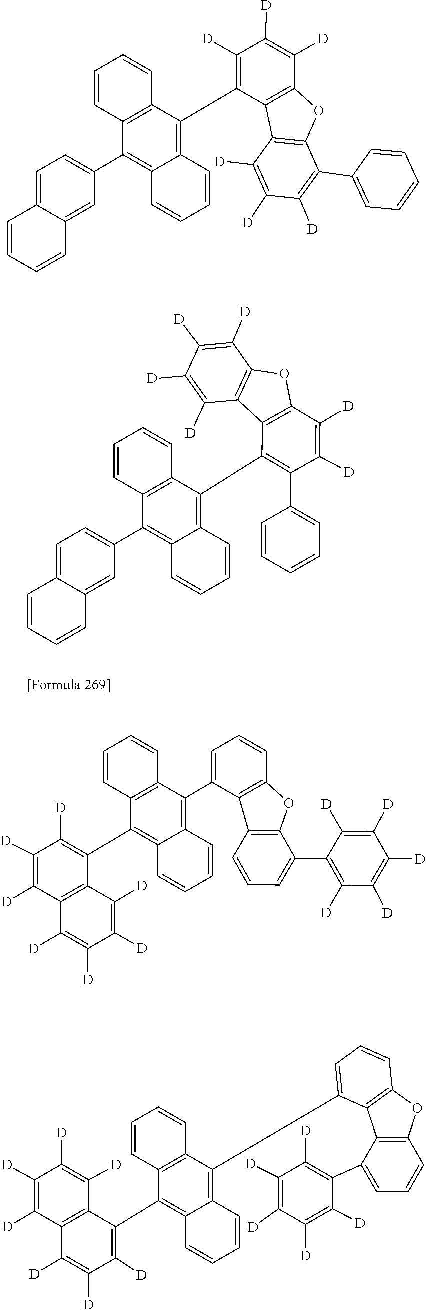 [SMILES: [2H]c1c([2H])c(-c2c3ccccc3c(-c3ccc4ccccc4c3)c3ccccc23)c2c(oc3c(-c4ccccc4)c([2H])c([2H])c([2H])c32)c1[2H] ; [2H]c1c([2H])c([2H])c2c(oc3c([2H])c([2H])c(-c4ccccc4)c(-c4c5ccccc5c(-c5ccc6ccccc6c5)c5ccccc45)c32)c1[2H] ; [2H]c1c([2H])c([2H])c(-c2cccc3c2oc2cccc(-c4c5ccccc5c(-c5c([2H])c([2H])c([2H])c6c([2H])c([2H])c([2H])c([2H])c56)c5ccccc45)c23)c([2H])c1[2H] ; [2H]c1c([2H])c([2H])c(-c2cccc3oc4cccc(-c5c6ccccc6c(-c6c([2H])c([2H])c([2H])c7c([2H])c([2H])c([2H])c([2H])c67)c6ccccc56)c4c23)c([2H])c1[2H]]

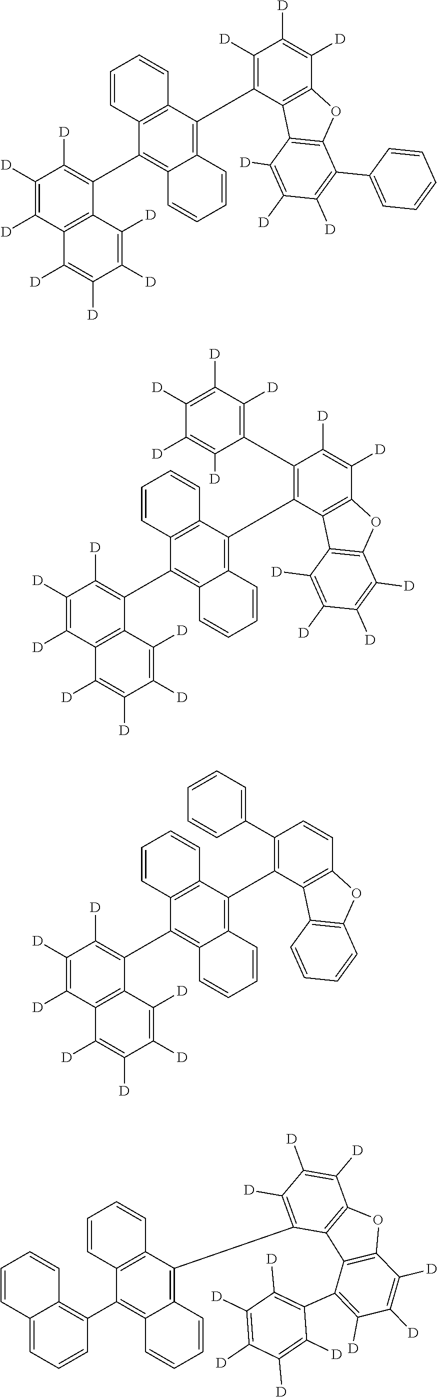

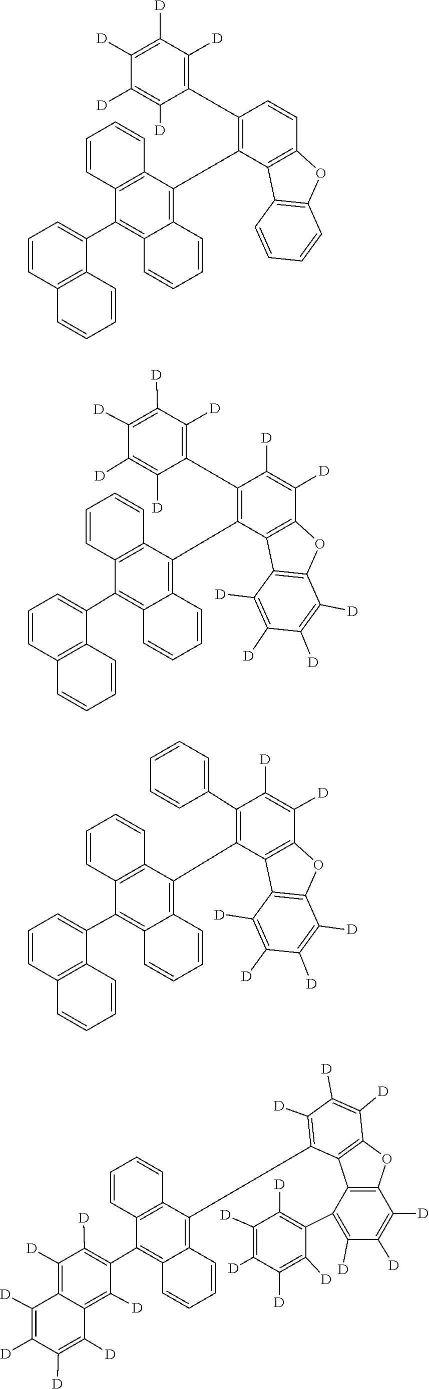

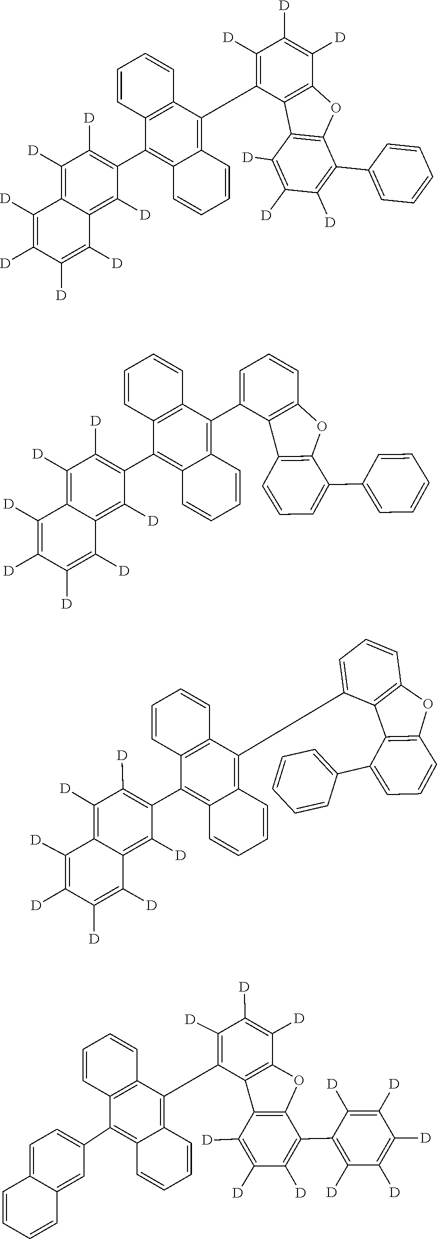

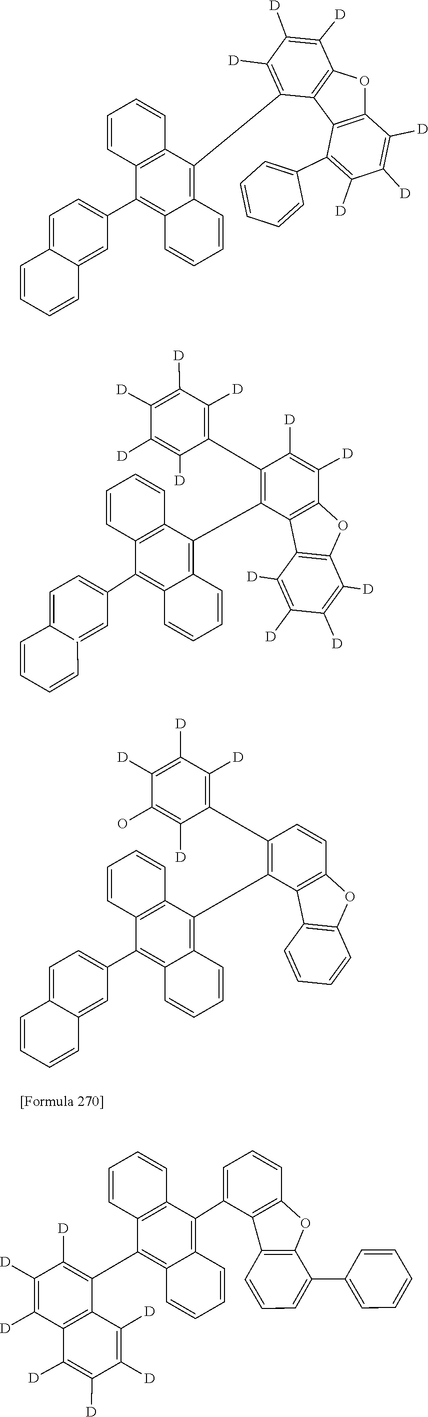

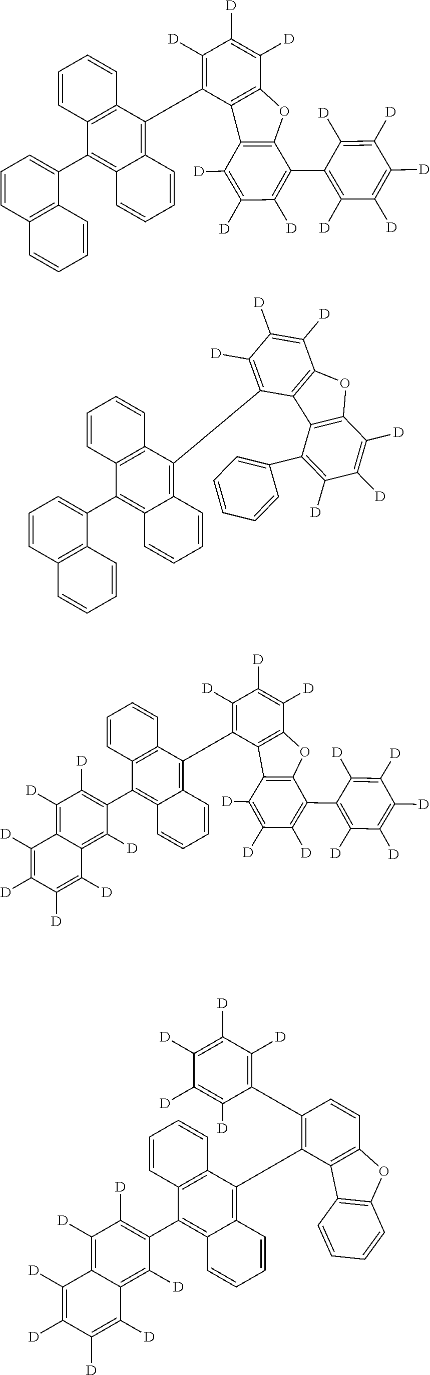

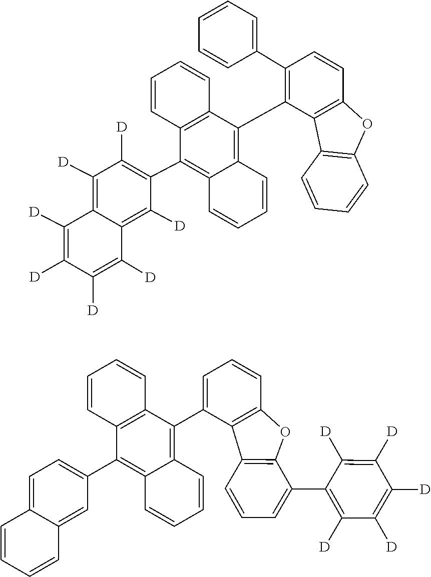 [SMILES: [2H]c1c([2H])c([2H])c2c([2H])c(-c3c4ccccc4c(-c4c(-c5ccccc5)ccc5oc6ccccc6c45)c4ccccc34)c([2H])c([2H])c2c1[2H] ; [2H]c1c([2H])c([2H])c(-c2cccc3c2oc2cccc(-c4c5ccccc5c(-c5ccc6ccccc6c5)c5ccccc45)c23)c([2H])c1[2H]]

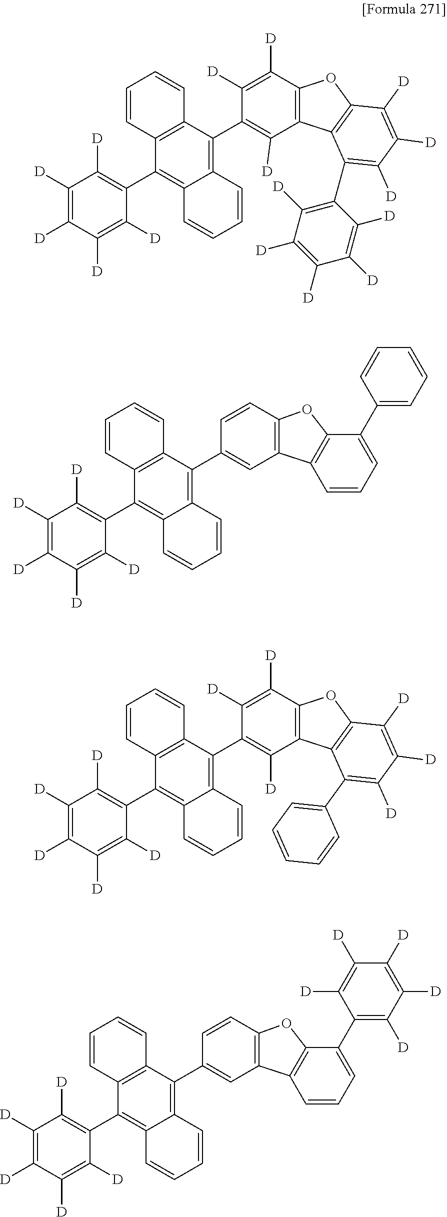 [SMILES: [2H]c1c([2H])c([2H])c(-c2c3ccccc3c(-c3c([2H])c([2H])c4oc5c([2H])c([2H])c([2H])c(-c6c([2H])c([2H])c([2H])c([2H])c6[2H])c5c4c3[2H])c3ccccc23)c([2H])c1[2H] ; [2H]c1c([2H])c([2H])c(-c2c3ccccc3c(-c3ccc4oc5c(-c6ccccc6)cccc5c4c3)c3ccccc23)c([2H])c1[2H] ; [2H]c1c([2H])c([2H])c(-c2c3ccccc3c(-c3c([2H])c([2H])c4oc5c([2H])c([2H])c([2H])c(-c6ccccc6)c5c4c3[2H])c3ccccc23)c([2H])c1[2H] ; [2H]c1c([2H])c([2H])c(-c2c3ccccc3c(-c3ccc4oc5c(-c6c([2H])c([2H])c([2H])c([2H])c6[2H])cccc5c4c3)c3ccccc23)c([2H])c1[2H]]

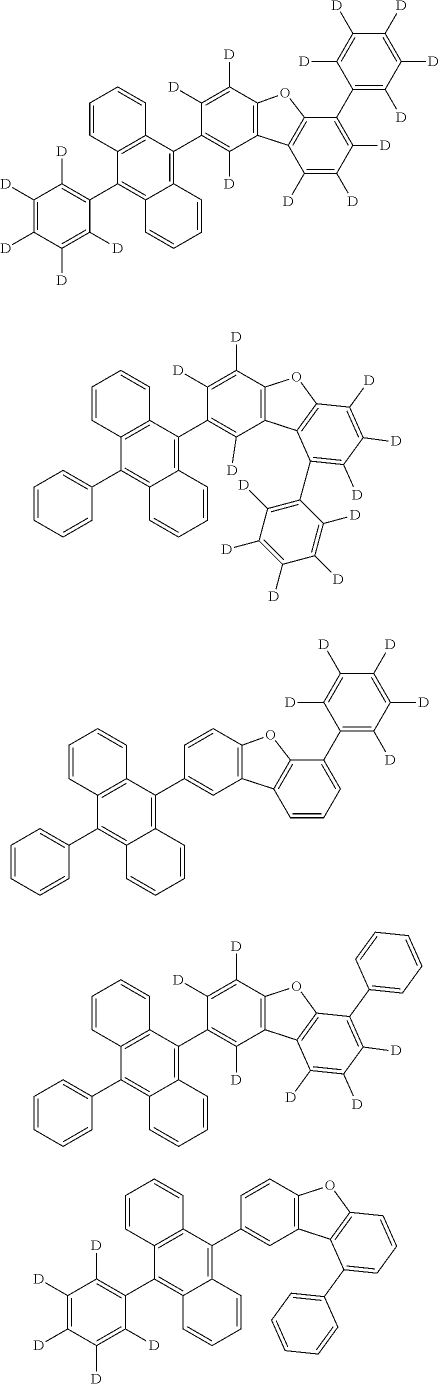

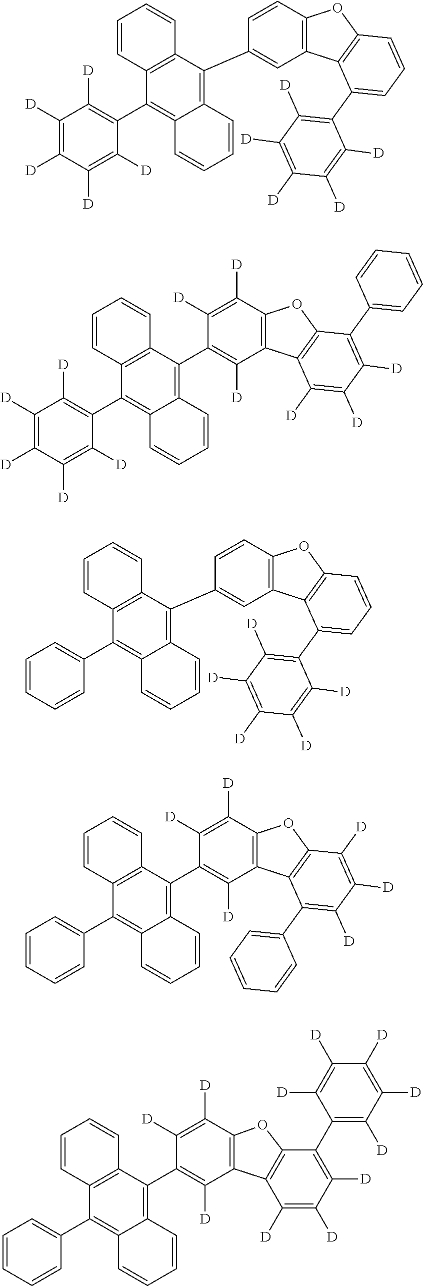

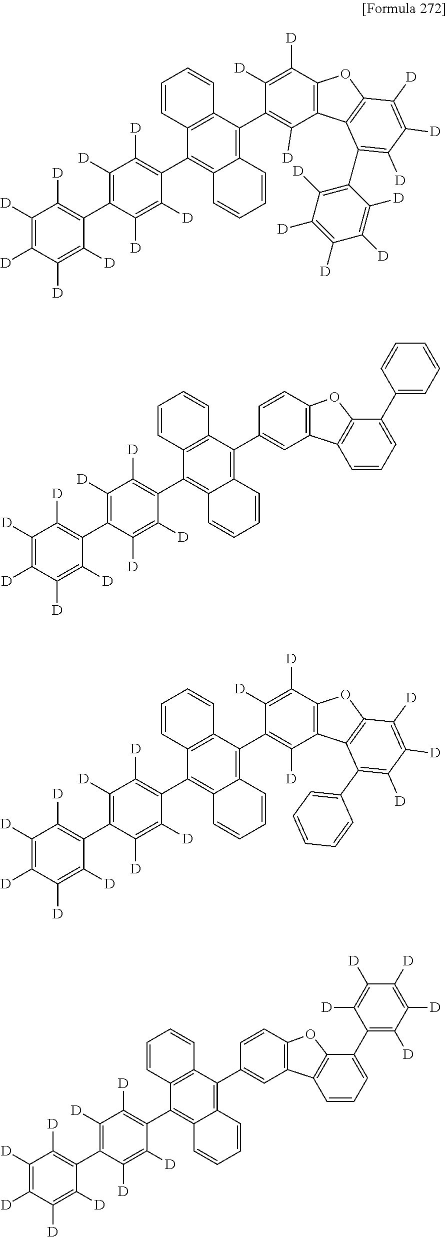

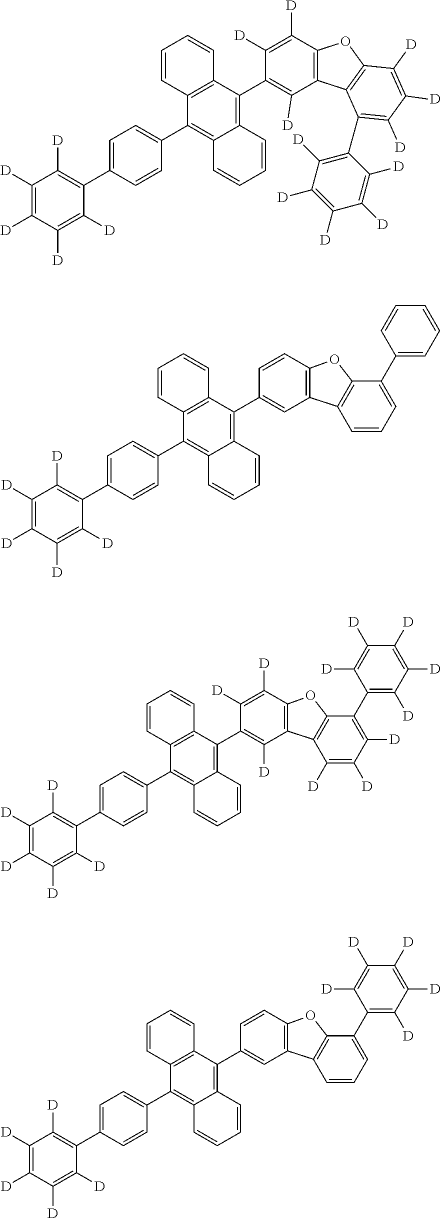

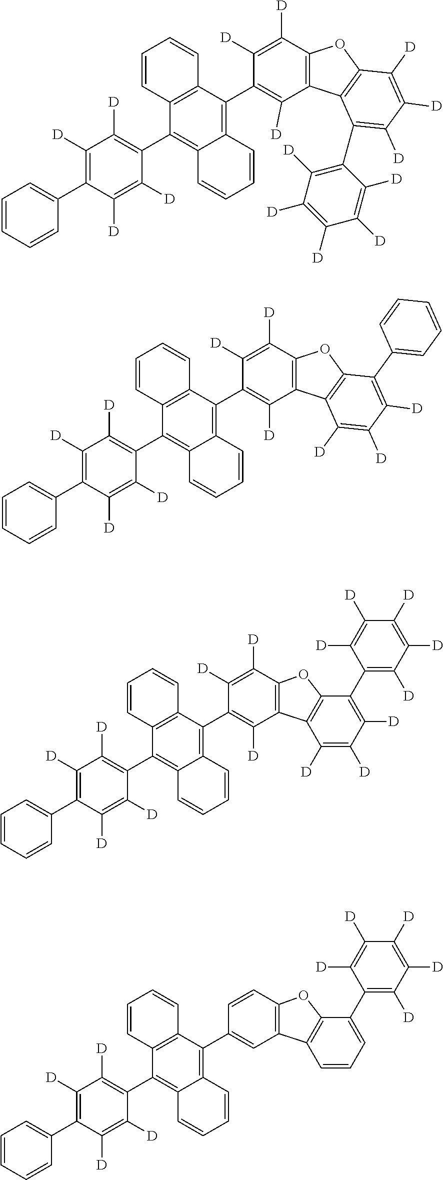

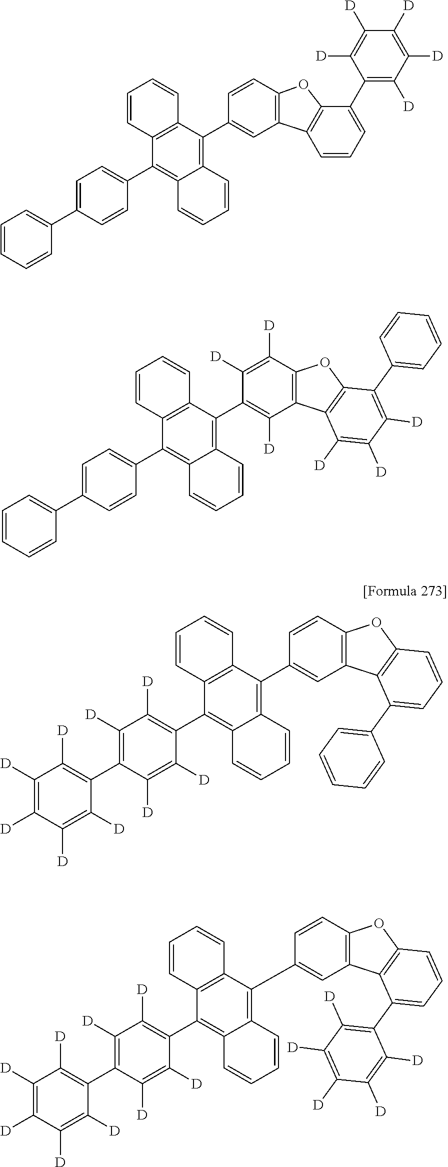 [SMILES: [2H]c1c([2H])c([2H])c(-c2cccc3c2oc2ccc(-c4c5ccccc5c(-c5ccc(-c6ccccc6)cc5)c5ccccc45)cc23)c([2H])c1[2H] ; [2H]c1c(-c2c3ccccc3c(-c3ccc(-c4ccccc4)cc3)c3ccccc23)c([2H])c2c(oc3c(-c4ccccc4)c([2H])c([2H])c([2H])c32)c1[2H] ; [2H]c1c([2H])c([2H])c(-c2c([2H])c([2H])c(-c3c4ccccc4c(-c4ccc5oc6cccc(-c7ccccc7)c6c5c4)c4ccccc34)c([2H])c2[2H])c([2H])c1[2H] ; [2H]c1c([2H])c([2H])c(-c2c([2H])c([2H])c(-c3c4ccccc4c(-c4ccc5oc6cccc(-c7c([2H])c([2H])c([2H])c([2H])c7[2H])c6c5c4)c4ccccc34)c([2H])c2[2H])c([2H])c1[2H]]

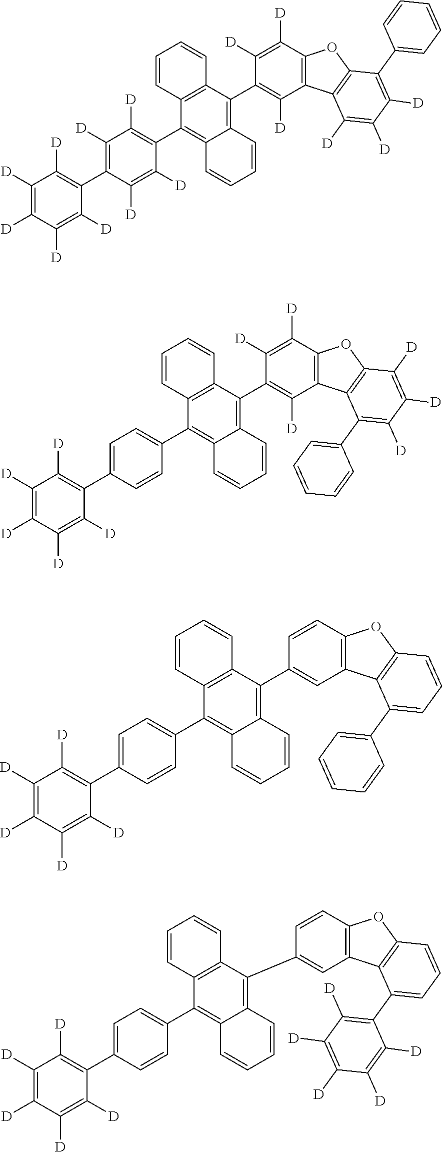

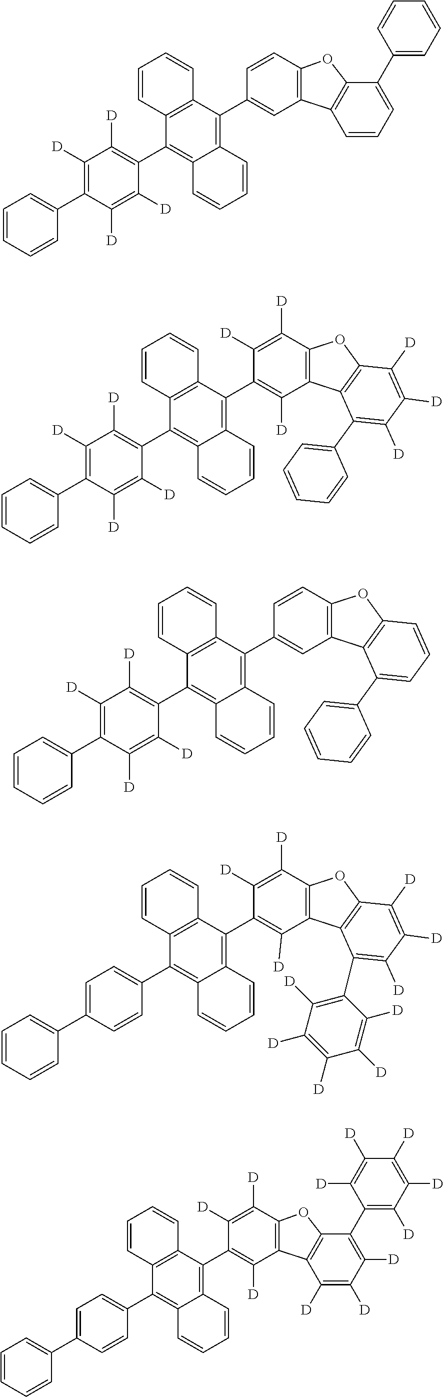

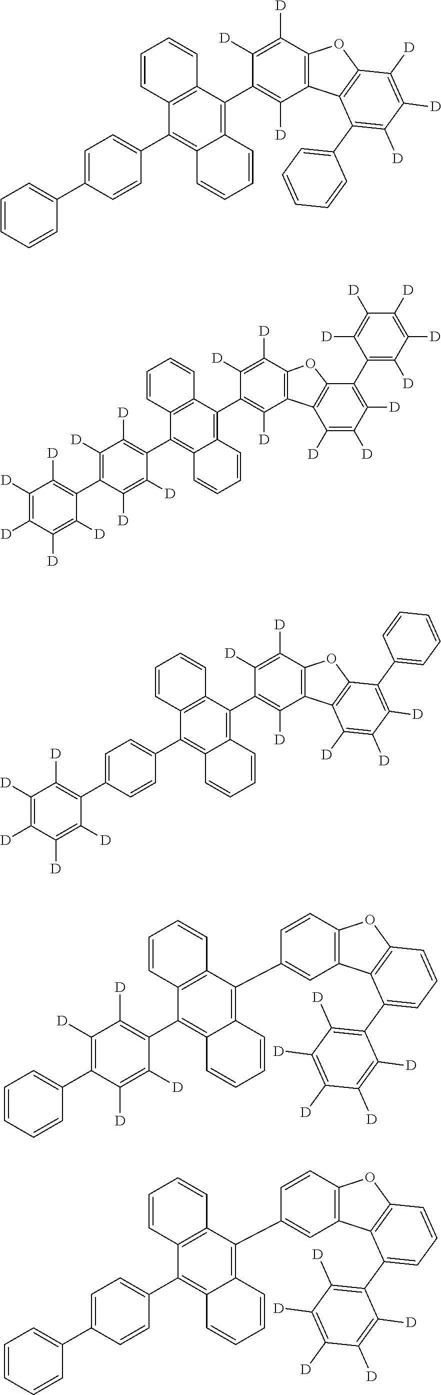

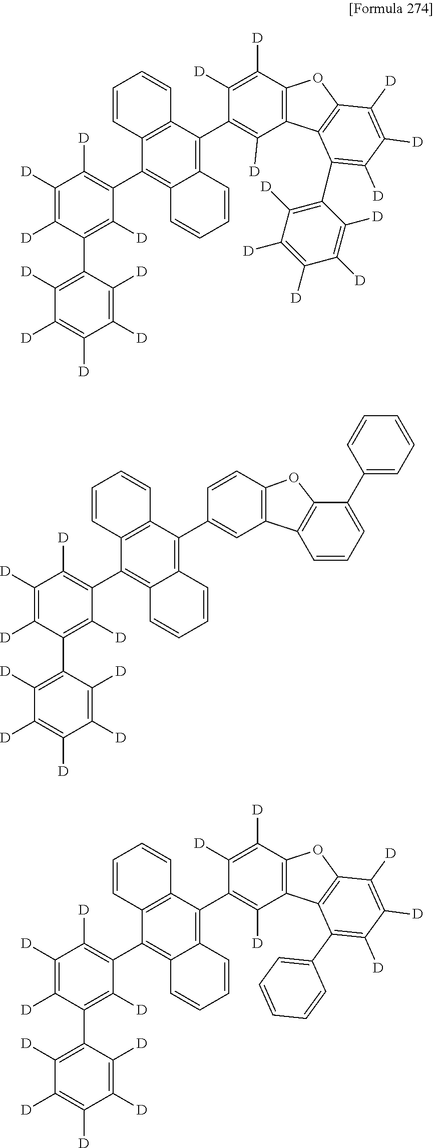 [SMILES: [2H]c1c([2H])c([2H])c(-c2c([2H])c([2H])c([2H])c(-c3c4ccccc4c(-c4c([2H])c([2H])c5oc6c([2H])c([2H])c([2H])c(-c7c([2H])c([2H])c([2H])c([2H])c7[2H])c6c5c4[2H])c4ccccc34)c2[2H])c([2H])c1[2H] ; [2H]c1c([2H])c([2H])c(-c2c([2H])c([2H])c([2H])c(-c3c4ccccc4c(-c4ccc5oc6c(-c7ccccc7)cccc6c5c4)c4ccccc34)c2[2H])c([2H])c1[2H] ; [2H]c1c([2H])c([2H])c(-c2c([2H])c([2H])c([2H])c(-c3c4ccccc4c(-c4c([2H])c([2H])c5oc6c([2H])c([2H])c([2H])c(-c7ccccc7)c6c5c4[2H])c4ccccc34)c2[2H])c([2H])c1[2H]]

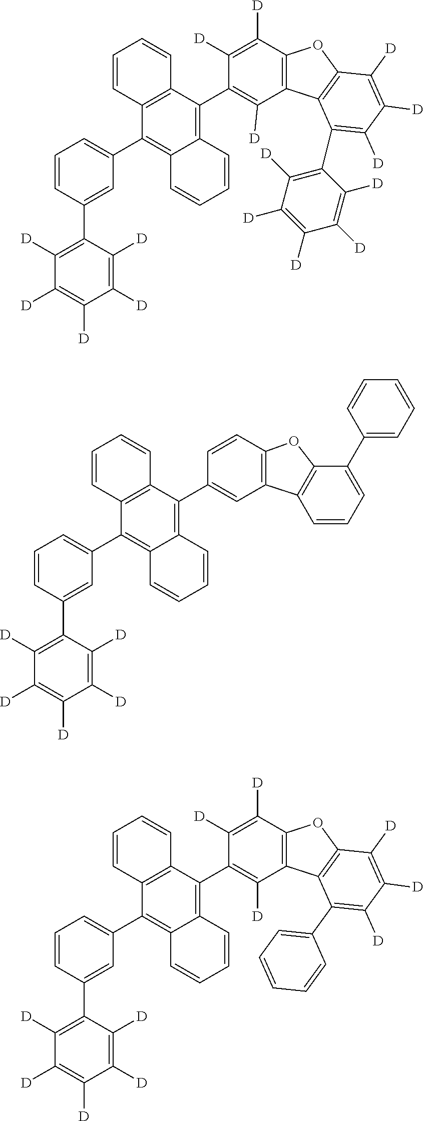 [SMILES: [2H]c1c([2H])c([2H])c(-c2cccc(-c3c4ccccc4c(-c4c([2H])c([2H])c5oc6c([2H])c([2H])c([2H])c(-c7c([2H])c([2H])c([2H])c([2H])c7[2H])c6c5c4[2H])c4ccccc34)c2)c([2H])c1[2H] ; [2H]c1c([2H])c([2H])c(-c2cccc(-c3c4ccccc4c(-c4ccc5oc6c(-c7ccccc7)cccc6c5c4)c4ccccc34)c2)c([2H])c1[2H] ; [2H]c1c([2H])c([2H])c(-c2cccc(-c3c4ccccc4c(-c4c([2H])c([2H])c5oc6c([2H])c([2H])c([2H])c(-c7ccccc7)c6c5c4[2H])c4ccccc34)c2)c([2H])c1[2H]]

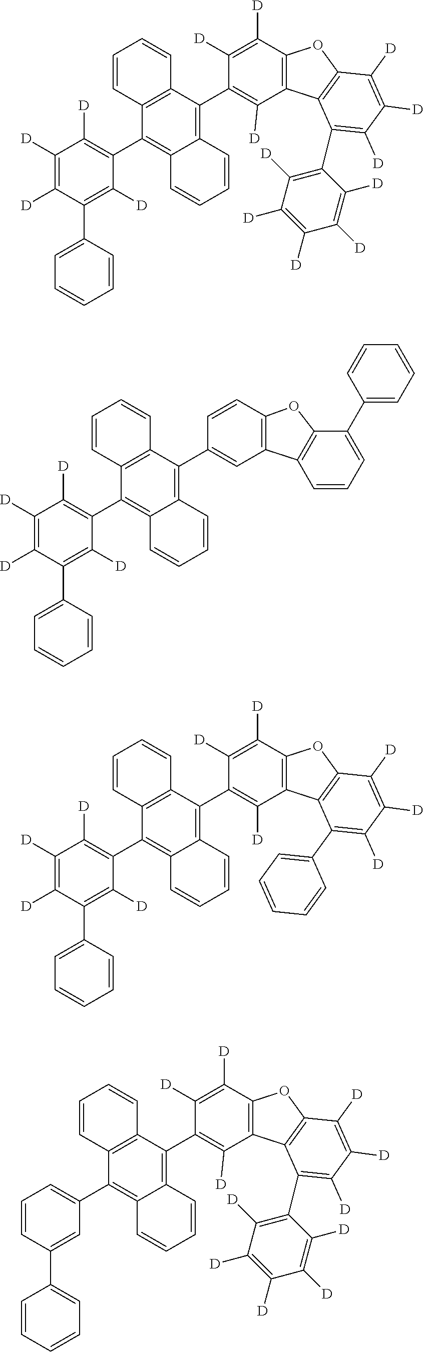

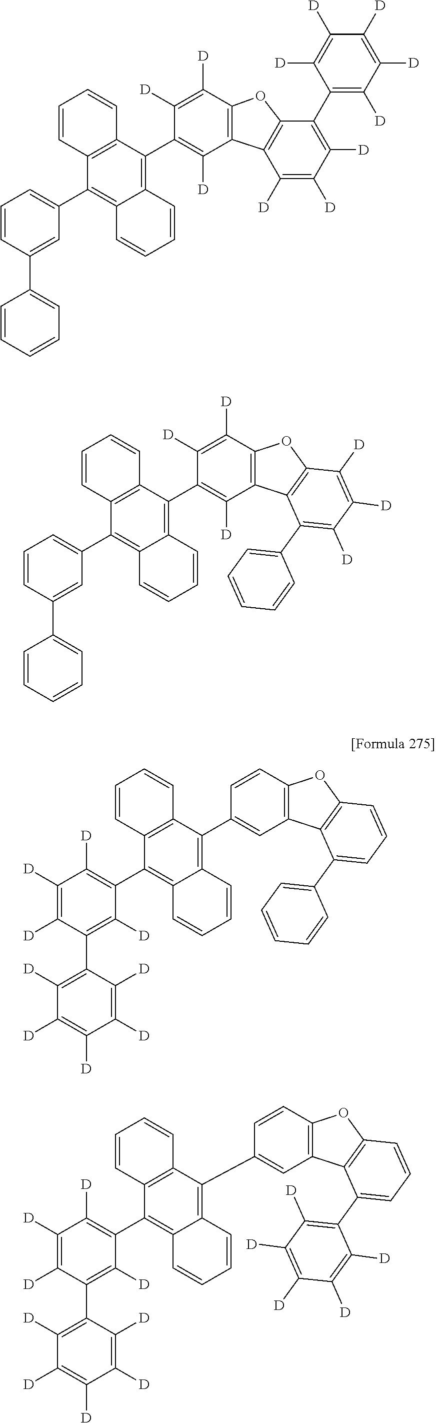

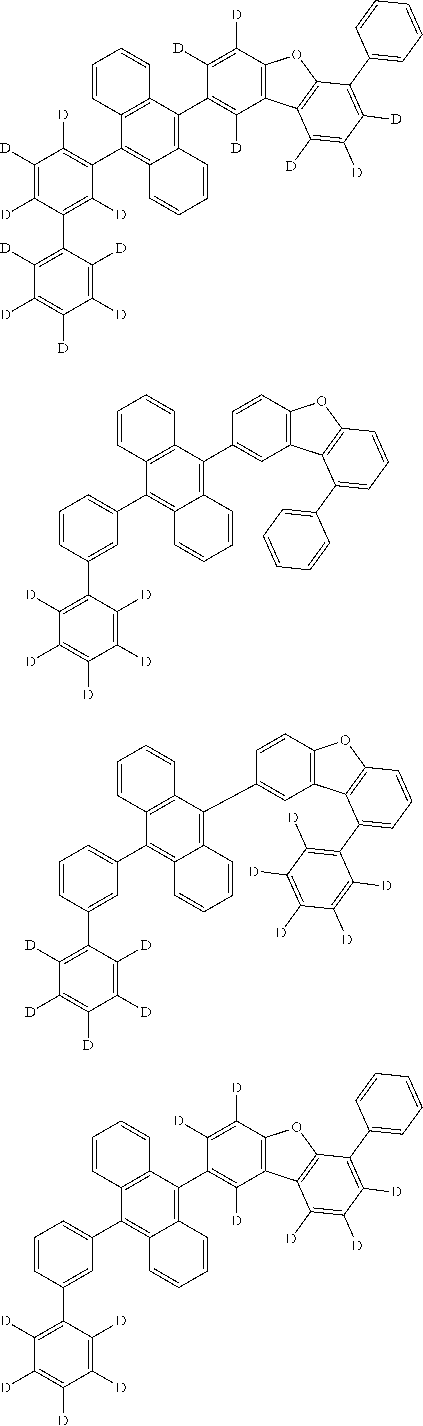

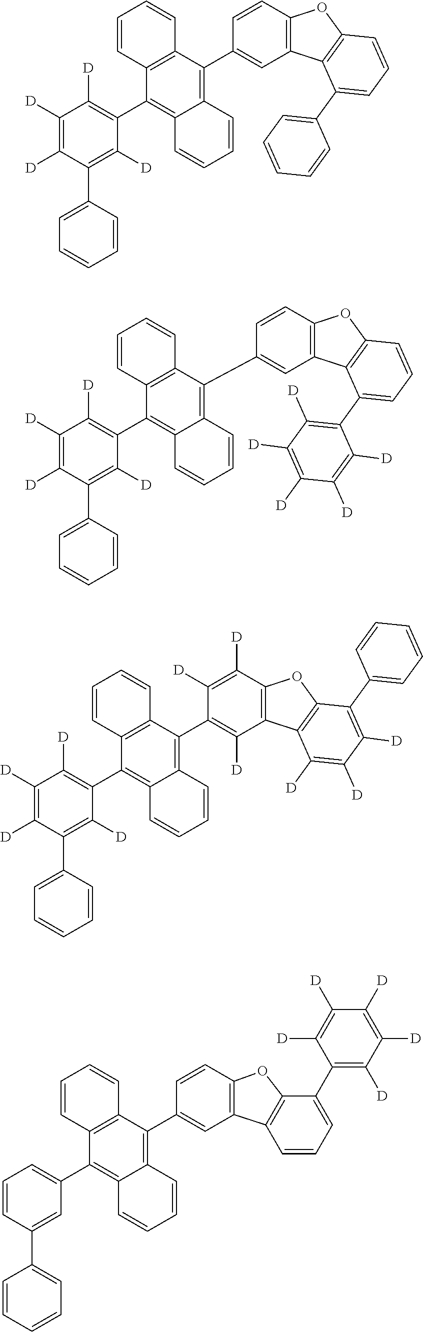 [SMILES: [2H]c1c([2H])c(-c2ccccc2)c([2H])c(-c2c3ccccc3c(-c3ccc4oc5cccc(-c6ccccc6)c5c4c3)c3ccccc23)c1[2H] ; [2H]c1c([2H])c([2H])c(-c2cccc3oc4ccc(-c5c6ccccc6c(-c6c([2H])c([2H])c([2H])c(-c7ccccc7)c6[2H])c6ccccc56)cc4c23)c([2H])c1[2H] ; [2H]c1c([2H])c(-c2ccccc2)c([2H])c(-c2c3ccccc3c(-c3c([2H])c([2H])c4oc5c(-c6ccccc6)c([2H])c([2H])c([2H])c5c4c3[2H])c3ccccc23)c1[2H] ; [2H]c1c([2H])c([2H])c(-c2cccc3c2oc2ccc(-c4c5ccccc5c(-c5cccc(-c6ccccc6)c5)c5ccccc45)cc23)c([2H])c1[2H]]

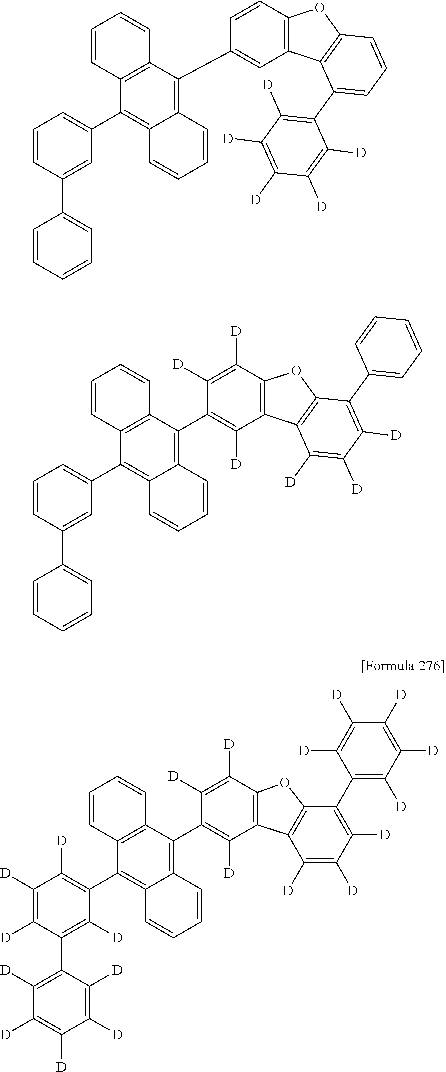 [SMILES: [2H]c1c([2H])c([2H])c(-c2cccc3oc4ccc(-c5c6ccccc6c(-c6cccc(-c7ccccc7)c6)c6ccccc56)cc4c23)c([2H])c1[2H] ; [2H]c1c(-c2c3ccccc3c(-c3cccc(-c4ccccc4)c3)c3ccccc23)c([2H])c2c(oc3c(-c4ccccc4)c([2H])c([2H])c([2H])c32)c1[2H] ; [2H]c1c([2H])c([2H])c(-c2c([2H])c([2H])c([2H])c(-c3c4ccccc4c(-c4c([2H])c([2H])c5oc6c(-c7c([2H])c([2H])c([2H])c([2H])c7[2H])c([2H])c([2H])c([2H])c6c5c4[2H])c4ccccc34)c2[2H])c([2H])c1[2H]]

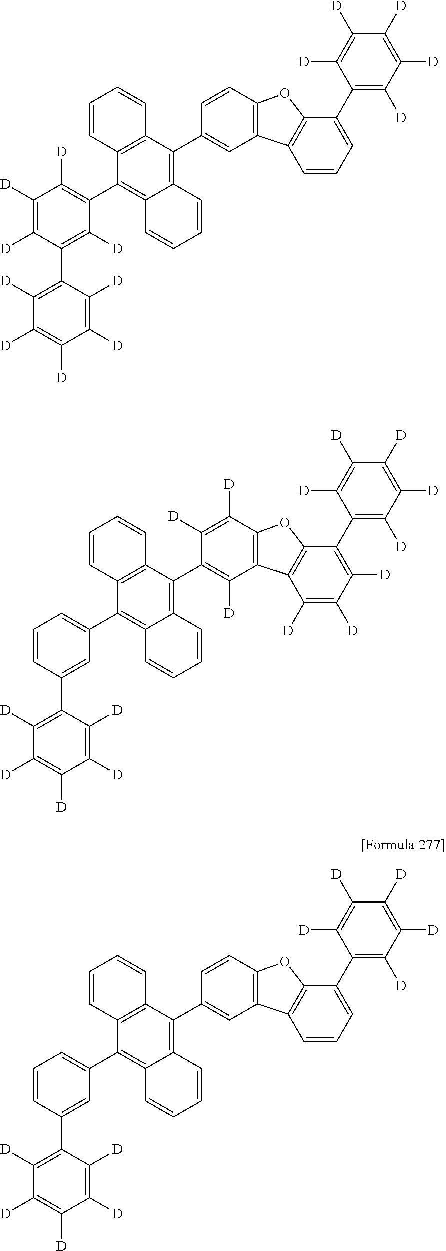 [SMILES: [2H]c1c([2H])c([2H])c(-c2c([2H])c([2H])c([2H])c(-c3c4ccccc4c(-c4ccc5oc6c(-c7c([2H])c([2H])c([2H])c([2H])c7[2H])cccc6c5c4)c4ccccc34)c2[2H])c([2H])c1[2H] ; [2H]c1c([2H])c([2H])c(-c2cccc(-c3c4ccccc4c(-c4c([2H])c([2H])c5oc6c(-c7c([2H])c([2H])c([2H])c([2H])c7[2H])c([2H])c([2H])c([2H])c6c5c4[2H])c4ccccc34)c2)c([2H])c1[2H] ; [2H]c1c([2H])c([2H])c(-c2cccc(-c3c4ccccc4c(-c4ccc5oc6c(-c7c([2H])c([2H])c([2H])c([2H])c7[2H])cccc6c5c4)c4ccccc34)c2)c([2H])c1[2H]]

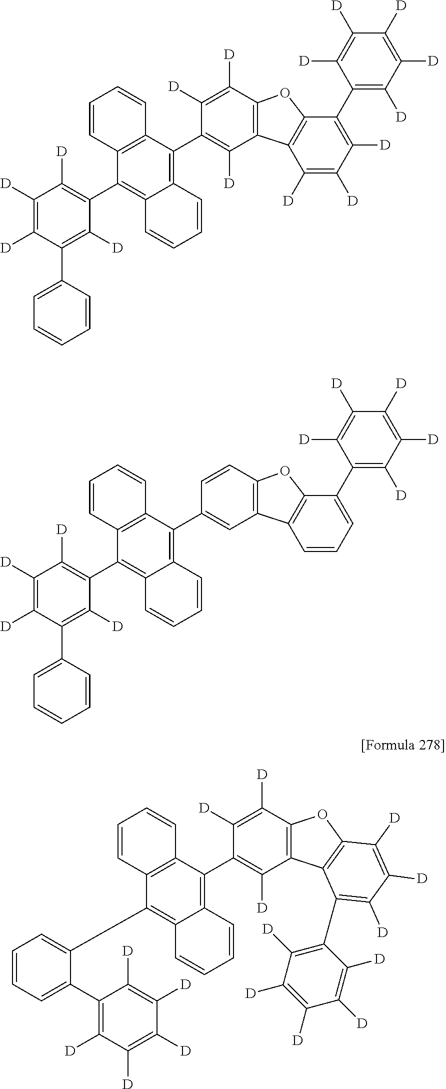 [SMILES: [2H]c1c([2H])c([2H])c(-c2c([2H])c([2H])c([2H])c3c2oc2c([2H])c([2H])c(-c4c5ccccc5c(-c5c([2H])c([2H])c([2H])c(-c6ccccc6)c5[2H])c5ccccc45)c([2H])c23)c([2H])c1[2H] ; [2H]c1c([2H])c([2H])c(-c2cccc3c2oc2ccc(-c4c5ccccc5c(-c5c([2H])c([2H])c([2H])c(-c6ccccc6)c5[2H])c5ccccc45)cc23)c([2H])c1[2H] ; [2H]c1c([2H])c([2H])c(-c2ccccc2-c2c3ccccc3c(-c3c([2H])c([2H])c4oc5c([2H])c([2H])c([2H])c(-c6c([2H])c([2H])c([2H])c([2H])c6[2H])c5c4c3[2H])c3ccccc23)c([2H])c1[2H]]

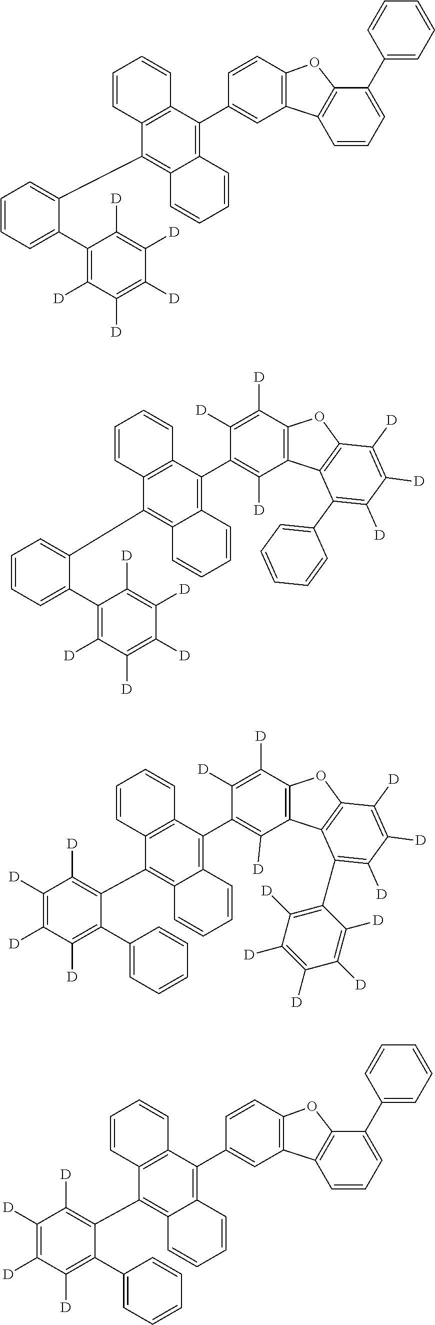

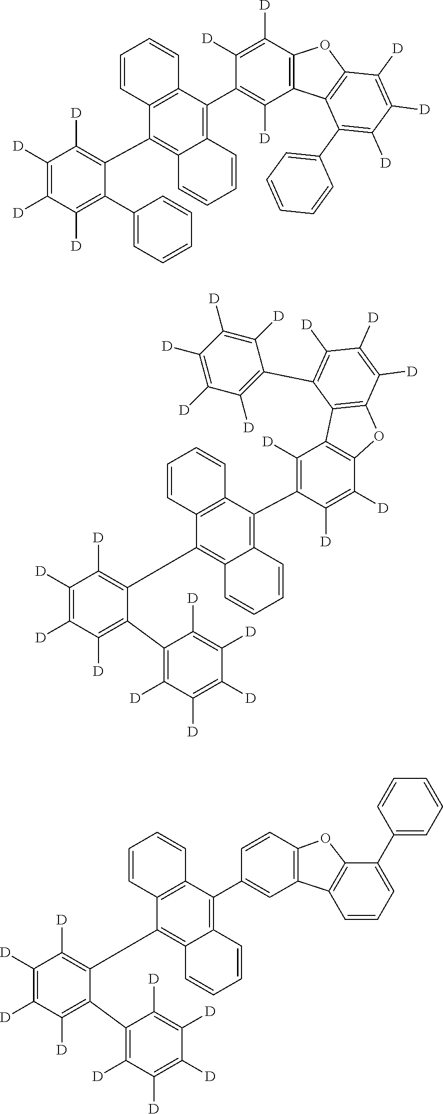 [SMILES: [2H]c1c([2H])c([2H])c(-c2c3ccccc3c(-c3c([2H])c([2H])c4oc5c([2H])c([2H])c([2H])c(-c6ccccc6)c5c4c3[2H])c3ccccc23)c(-c2ccccc2)c1[2H] ; [2H]c1c([2H])c([2H])c(-c2c([2H])c([2H])c([2H])c([2H])c2-c2c3ccccc3c(-c3c([2H])c([2H])c4oc5c([2H])c([2H])c([2H])c(-c6c([2H])c([2H])c([2H])c([2H])c6[2H])c5c4c3[2H])c3ccccc23)c([2H])c1[2H] ; [2H]c1c([2H])c([2H])c(-c2c([2H])c([2H])c([2H])c([2H])c2-c2c3ccccc3c(-c3ccc4oc5c(-c6ccccc6)cccc5c4c3)c3ccccc23)c([2H])c1[2H]]

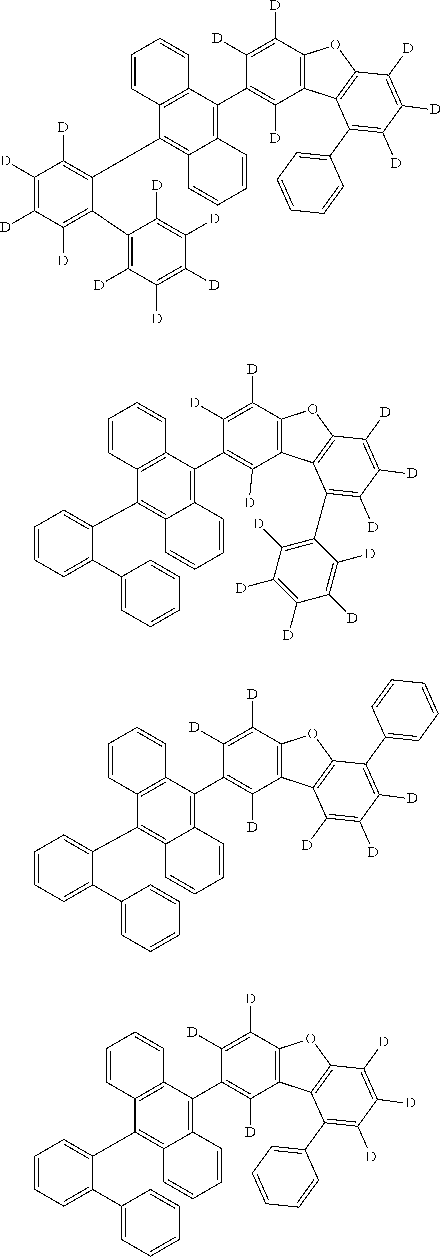

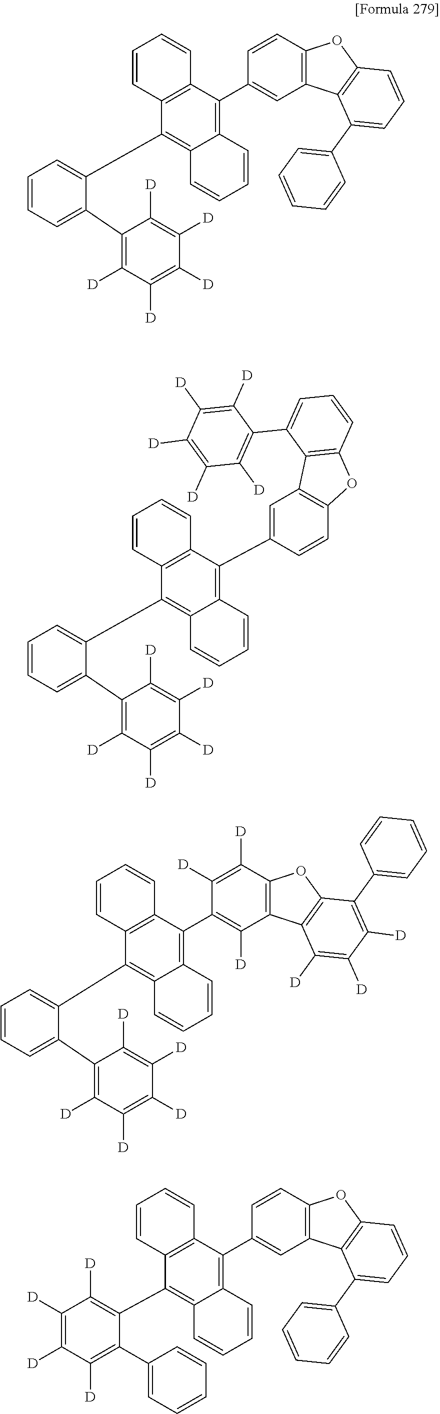 [SMILES: [2H]c1c([2H])c([2H])c(-c2ccccc2-c2c3ccccc3c(-c3ccc4oc5cccc(-c6ccccc6)c5c4c3)c3ccccc23)c([2H])c1[2H] ; [2H]c1c([2H])c([2H])c(-c2ccccc2-c2c3ccccc3c(-c3ccc4oc5cccc(-c6c([2H])c([2H])c([2H])c([2H])c6[2H])c5c4c3)c3ccccc23)c([2H])c1[2H] ; [2H]c1c([2H])c([2H])c(-c2ccccc2-c2c3ccccc3c(-c3c([2H])c([2H])c4oc5c(-c6ccccc6)c([2H])c([2H])c([2H])c5c4c3[2H])c3ccccc23)c([2H])c1[2H] ; [2H]c1c([2H])c([2H])c(-c2c3ccccc3c(-c3ccc4oc5cccc(-c6ccccc6)c5c4c3)c3ccccc23)c(-c2ccccc2)c1[2H]]

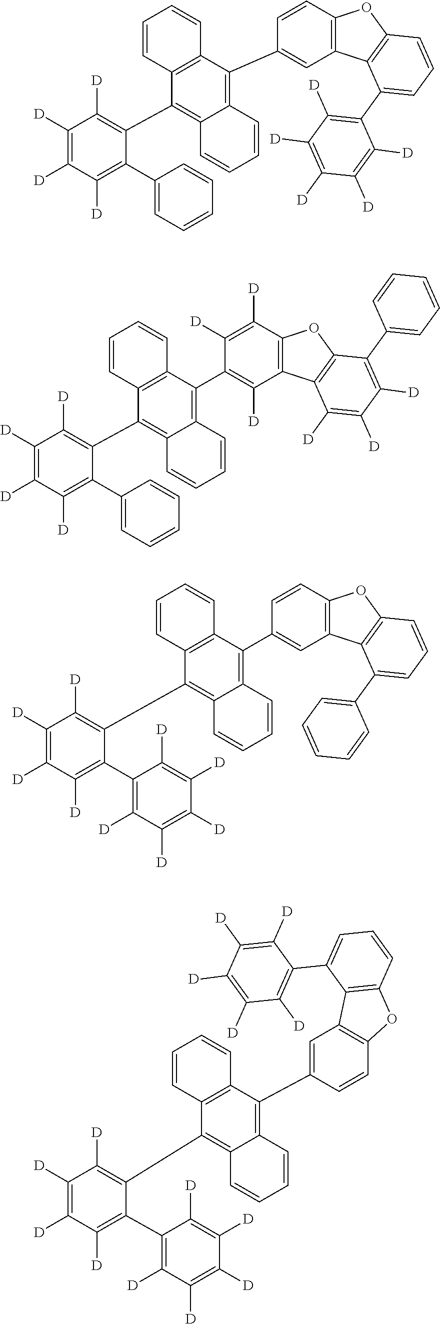

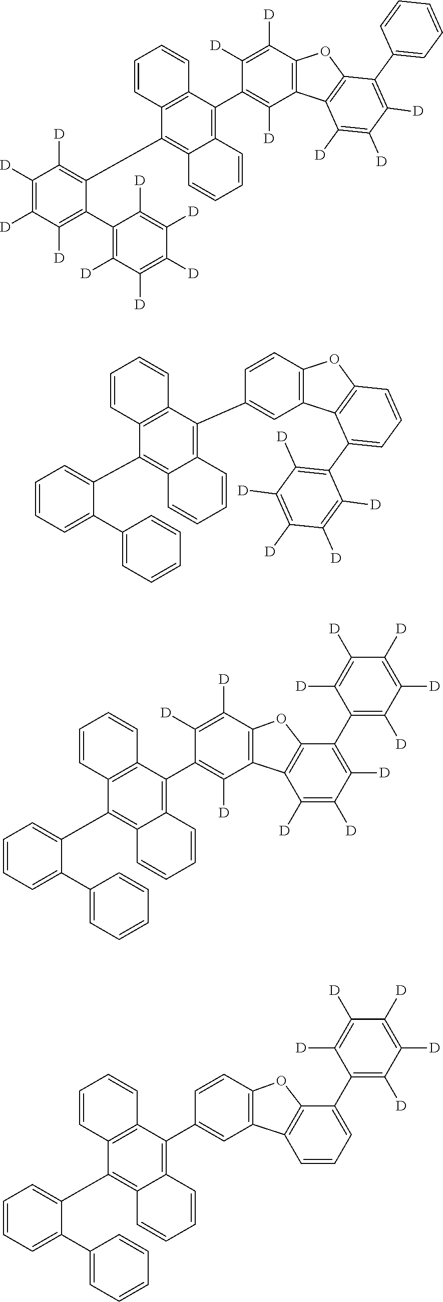 [SMILES: [2H]c1c([2H])c([2H])c(-c2c([2H])c([2H])c([2H])c([2H])c2-c2c3ccccc3c(-c3c([2H])c([2H])c4oc5c(-c6ccccc6)c([2H])c([2H])c([2H])c5c4c3[2H])c3ccccc23)c([2H])c1[2H] ; [2H]c1c([2H])c([2H])c(-c2cccc3oc4ccc(-c5c6ccccc6c(-c6ccccc6-c6ccccc6)c6ccccc56)cc4c23)c([2H])c1[2H] ; [2H]c1c([2H])c([2H])c(-c2c([2H])c([2H])c([2H])c3c2oc2c([2H])c([2H])c(-c4c5ccccc5c(-c5ccccc5-c5ccccc5)c5ccccc45)c([2H])c23)c([2H])c1[2H] ; [2H]c1c([2H])c([2H])c(-c2cccc3c2oc2ccc(-c4c5ccccc5c(-c5ccccc5-c5ccccc5)c5ccccc45)cc23)c([2H])c1[2H]]

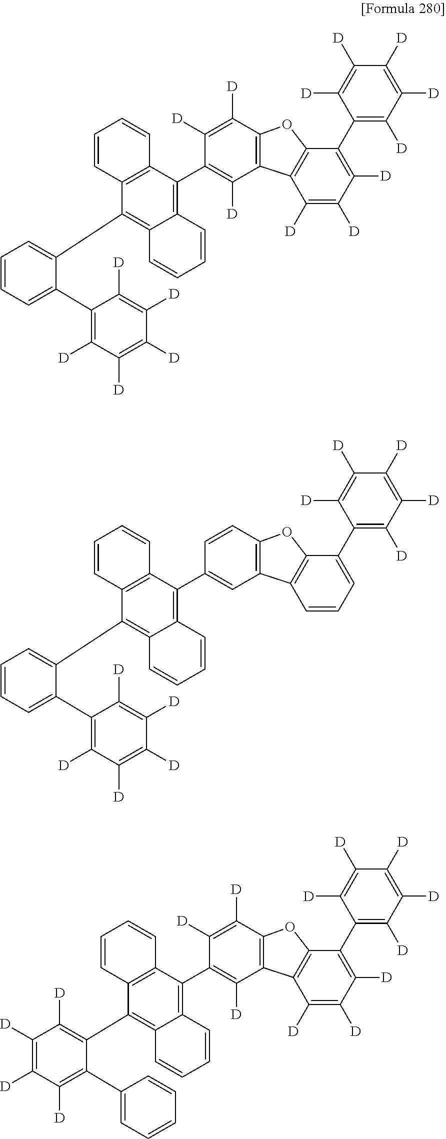 [SMILES: [2H]c1c([2H])c([2H])c(-c2ccccc2-c2c3ccccc3c(-c3c([2H])c([2H])c4oc5c(-c6c([2H])c([2H])c([2H])c([2H])c6[2H])c([2H])c([2H])c([2H])c5c4c3[2H])c3ccccc23)c([2H])c1[2H] ; [2H]c1c([2H])c([2H])c(-c2ccccc2-c2c3ccccc3c(-c3ccc4oc5c(-c6c([2H])c([2H])c([2H])c([2H])c6[2H])cccc5c4c3)c3ccccc23)c([2H])c1[2H] ; [2H]c1c([2H])c([2H])c(-c2c([2H])c([2H])c([2H])c3c2oc2c([2H])c([2H])c(-c4c5ccccc5c(-c5c([2H])c([2H])c([2H])c([2H])c5-c5ccccc5)c5ccccc45)c([2H])c23)c([2H])c1[2H]]

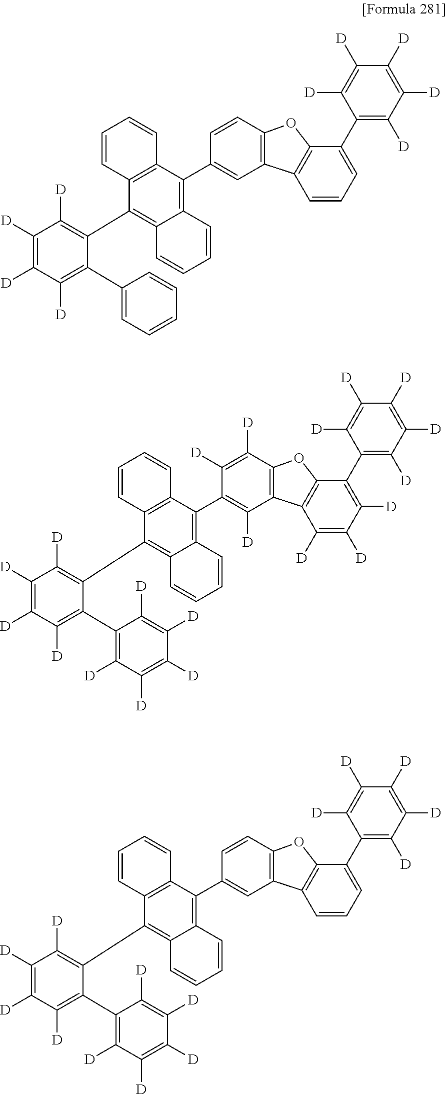 [SMILES: [2H]c1c([2H])c([2H])c(-c2cccc3c2oc2ccc(-c4c5ccccc5c(-c5c([2H])c([2H])c([2H])c([2H])c5-c5ccccc5)c5ccccc45)cc23)c([2H])c1[2H] ; [2H]c1c([2H])c([2H])c(-c2c([2H])c([2H])c([2H])c([2H])c2-c2c3ccccc3c(-c3c([2H])c([2H])c4oc5c(-c6c([2H])c([2H])c([2H])c([2H])c6[2H])c([2H])c([2H])c([2H])c5c4c3[2H])c3ccccc23)c([2H])c1[2H] ; [2H]c1c([2H])c([2H])c(-c2c([2H])c([2H])c([2H])c([2H])c2-c2c3ccccc3c(-c3ccc4oc5c(-c6c([2H])c([2H])c([2H])c([2H])c6[2H])cccc5c4c3)c3ccccc23)c([2H])c1[2H]]

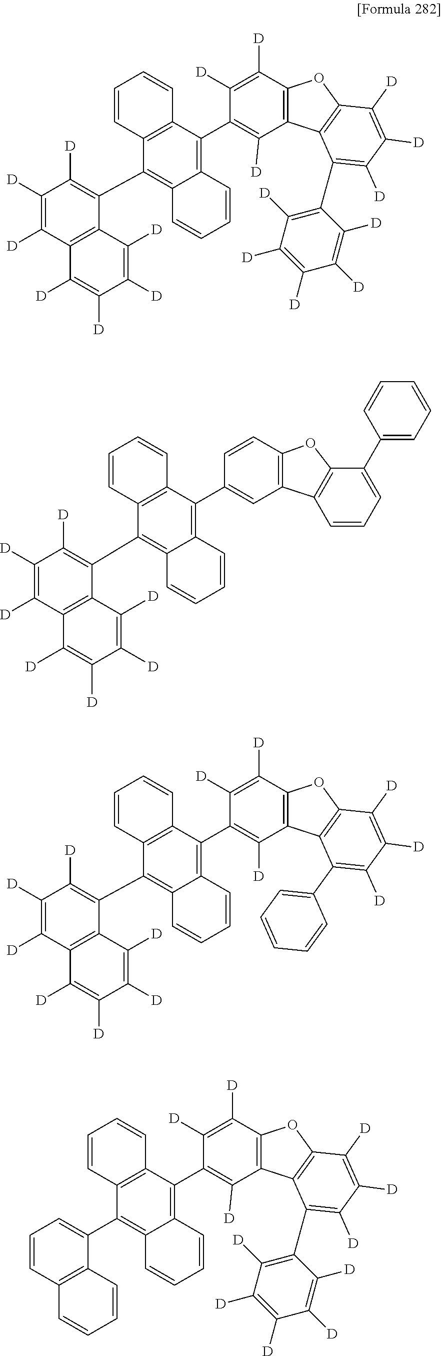

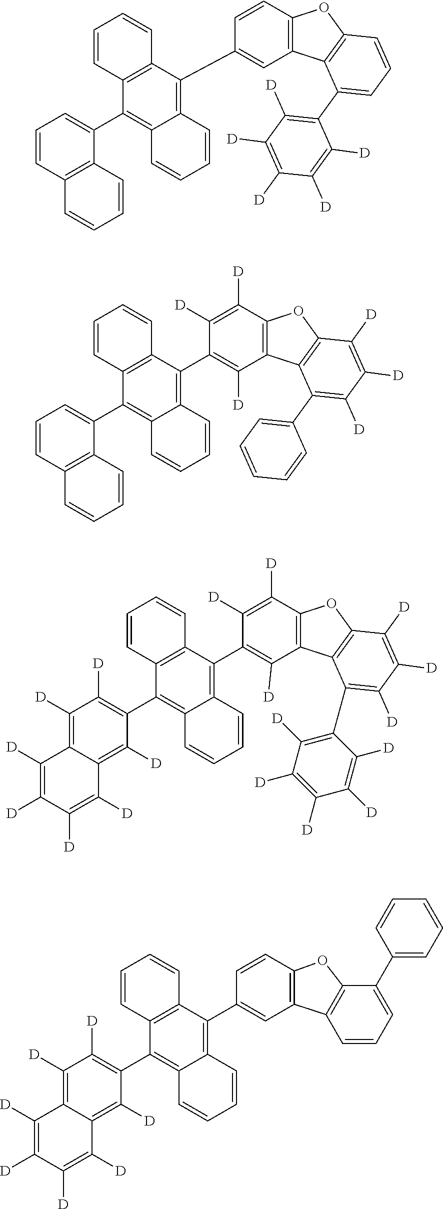 [SMILES: [2H]c1c([2H])c([2H])c(-c2cccc3oc4ccc(-c5c6ccccc6c(-c6cccc7ccccc67)c6ccccc56)cc4c23)c([2H])c1[2H] ; [2H]c1c(-c2c3ccccc3c(-c3cccc4ccccc34)c3ccccc23)c([2H])c2c(oc3c([2H])c([2H])c([2H])c(-c4ccccc4)c32)c1[2H] ; [2H]c1c([2H])c([2H])c(-c2c([2H])c([2H])c([2H])c3oc4c([2H])c([2H])c(-c5c6ccccc6c(-c6c([2H])c([2H])c7c([2H])c([2H])c([2H])c([2H])c7c6[2H])c6ccccc56)c([2H])c4c23)c([2H])c1[2H] ; [2H]c1c([2H])c([2H])c2c([2H])c(-c3c4ccccc4c(-c4ccc5oc6c(-c7ccccc7)cccc6c5c4)c4ccccc34)c([2H])c([2H])c2c1[2H]]

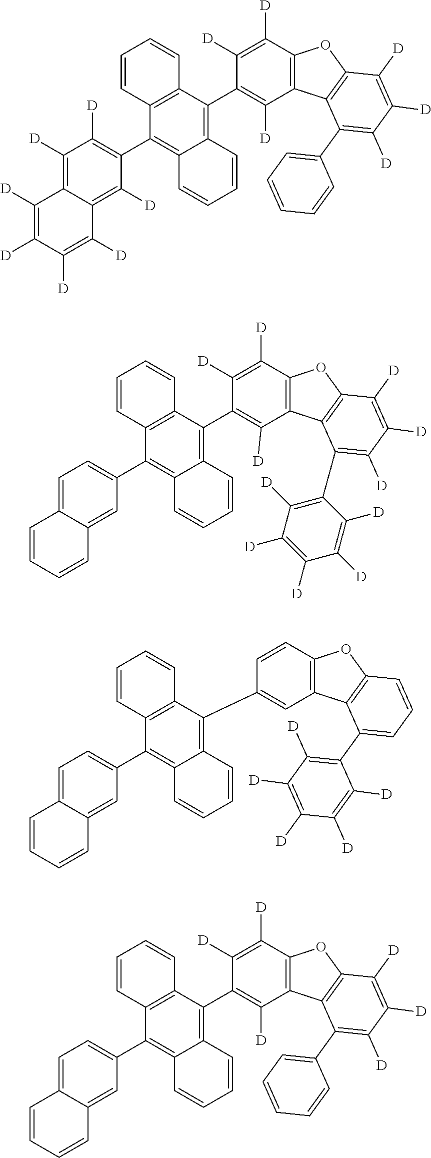

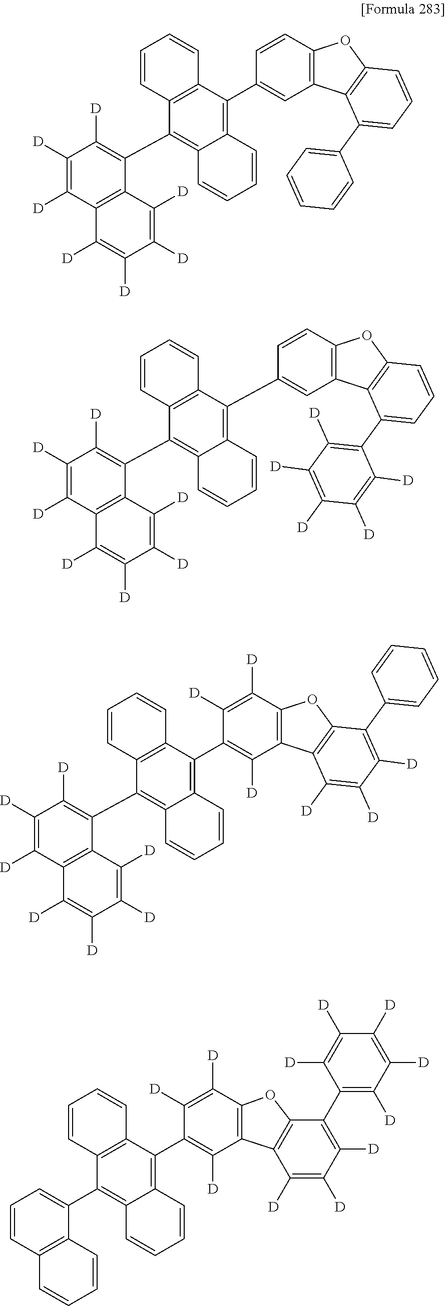

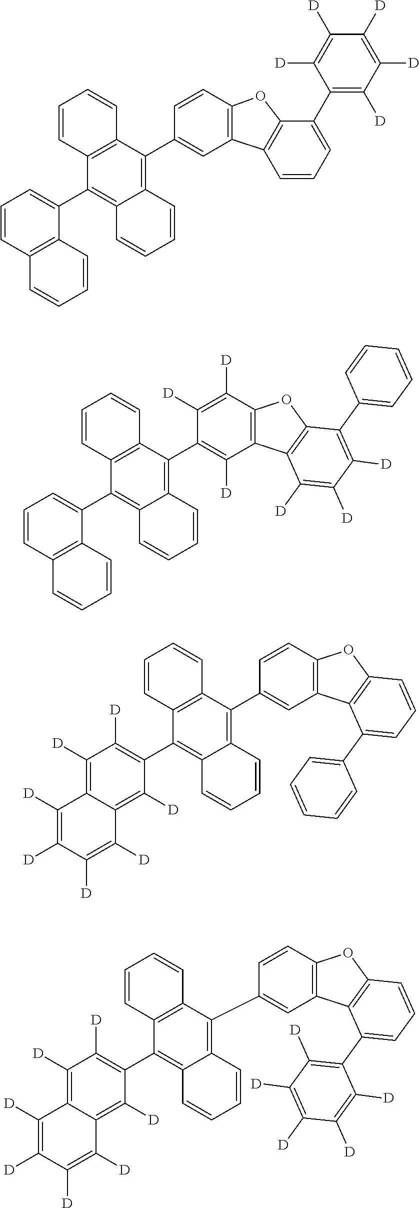 [SMILES: [2H]c1c([2H])c([2H])c(-c2cccc3c2oc2ccc(-c4c5ccccc5c(-c5cccc6ccccc56)c5ccccc45)cc23)c([2H])c1[2H] ; [2H]c1c(-c2c3ccccc3c(-c3cccc4ccccc34)c3ccccc23)c([2H])c2c(oc3c(-c4ccccc4)c([2H])c([2H])c([2H])c32)c1[2H] ; [2H]c1c([2H])c([2H])c2c([2H])c(-c3c4ccccc4c(-c4ccc5oc6cccc(-c7ccccc7)c6c5c4)c4ccccc34)c([2H])c([2H])c2c1[2H] ; [2H]c1c([2H])c([2H])c(-c2cccc3oc4ccc(-c5c6ccccc6c(-c6c([2H])c([2H])c7c([2H])c([2H])c([2H])c([2H])c7c6[2H])c6ccccc56)cc4c23)c([2H])c1[2H]]

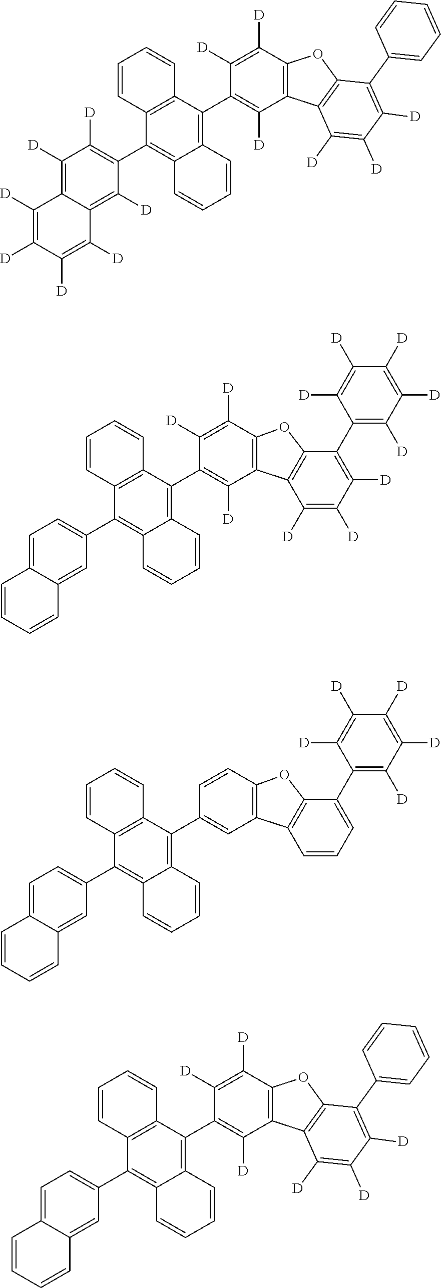 [SMILES: [2H]c1c(-c2c3ccccc3c(-c3c([2H])c([2H])c4c([2H])c([2H])c([2H])c([2H])c4c3[2H])c3ccccc23)c([2H])c2c(oc3c(-c4ccccc4)c([2H])c([2H])c([2H])c32)c1[2H] ; [2H]c1c([2H])c([2H])c(-c2c([2H])c([2H])c([2H])c3c2oc2c([2H])c([2H])c(-c4c5ccccc5c(-c5ccc6ccccc6c5)c5ccccc45)c([2H])c23)c([2H])c1[2H] ; [2H]c1c([2H])c([2H])c(-c2cccc3c2oc2ccc(-c4c5ccccc5c(-c5ccc6ccccc6c5)c5ccccc45)cc23)c([2H])c1[2H] ; [2H]c1c(-c2c3ccccc3c(-c3ccc4ccccc4c3)c3ccccc23)c([2H])c2c(oc3c(-c4ccccc4)c([2H])c([2H])c([2H])c32)c1[2H]]

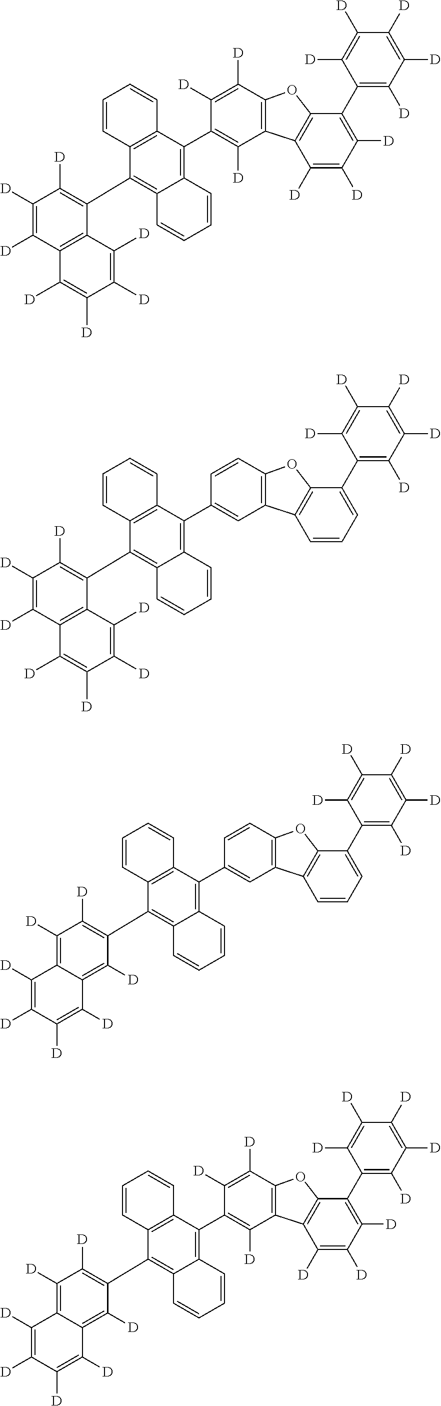

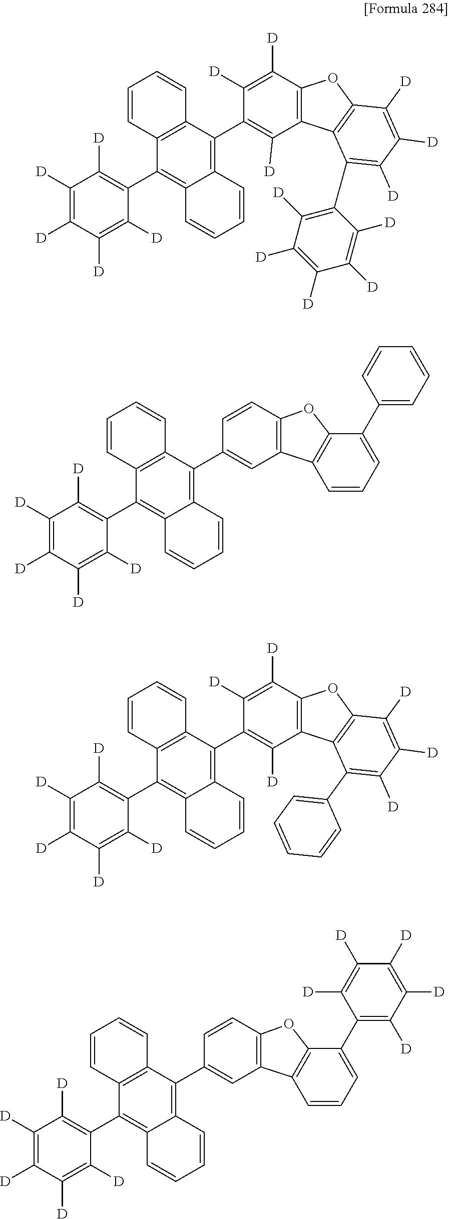

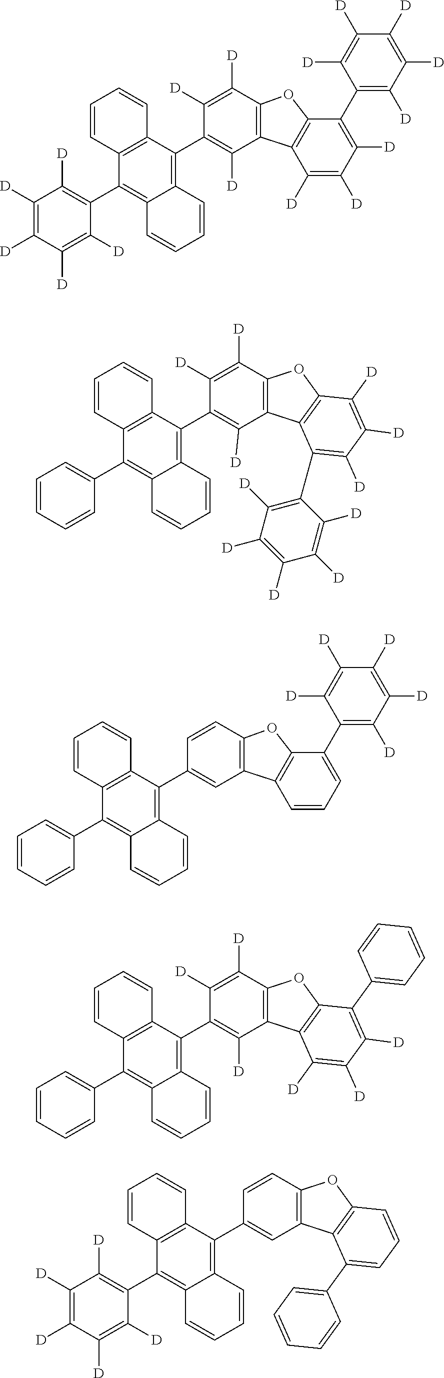

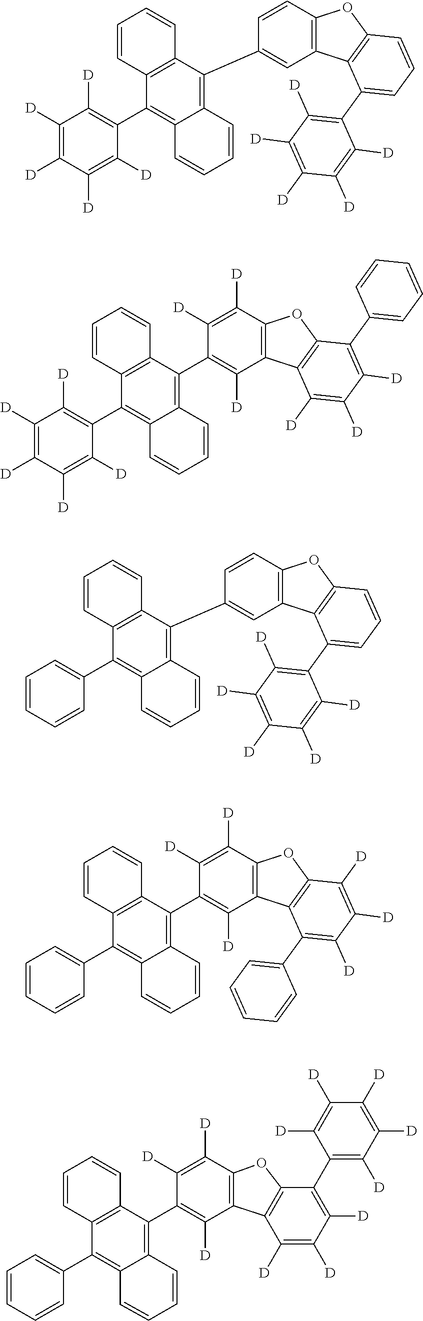

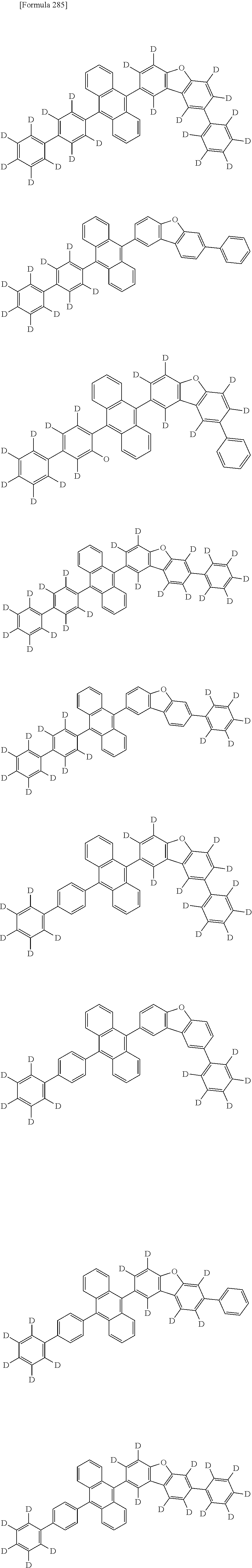

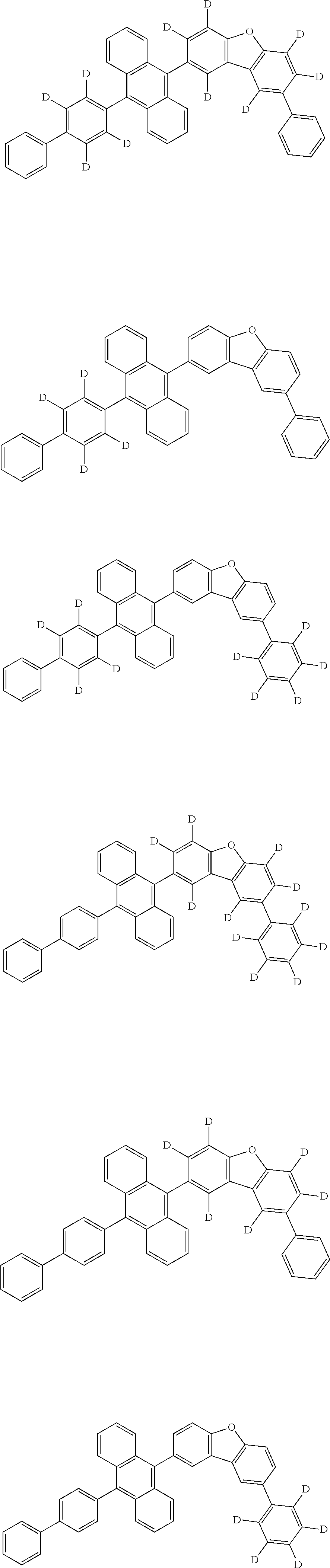

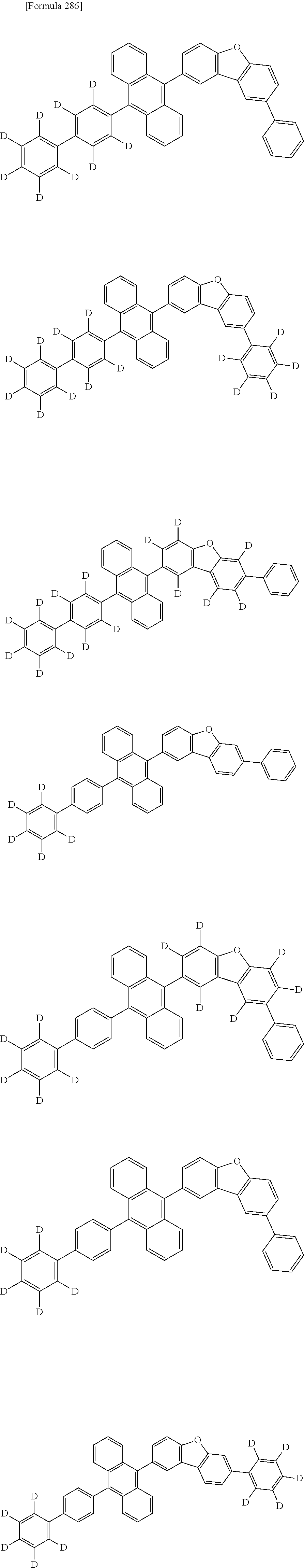

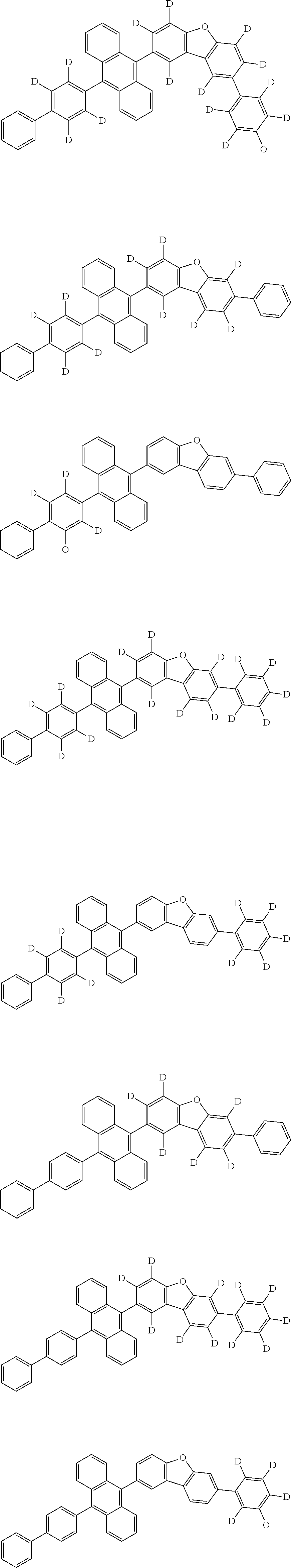

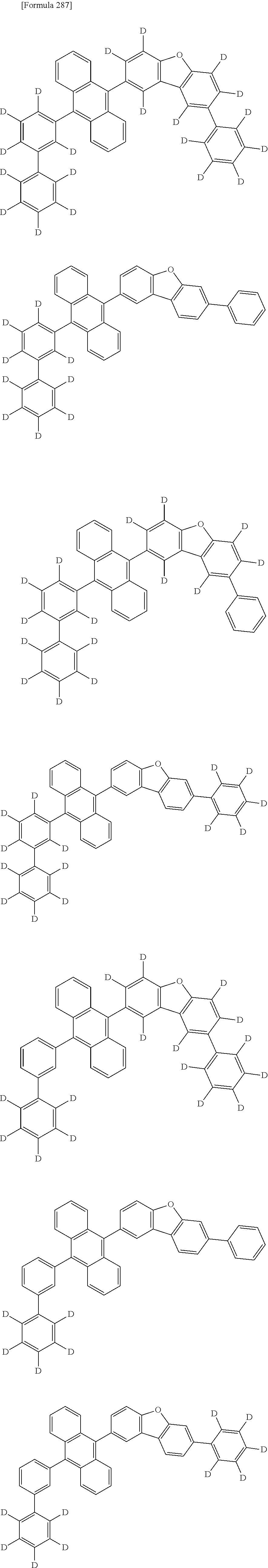

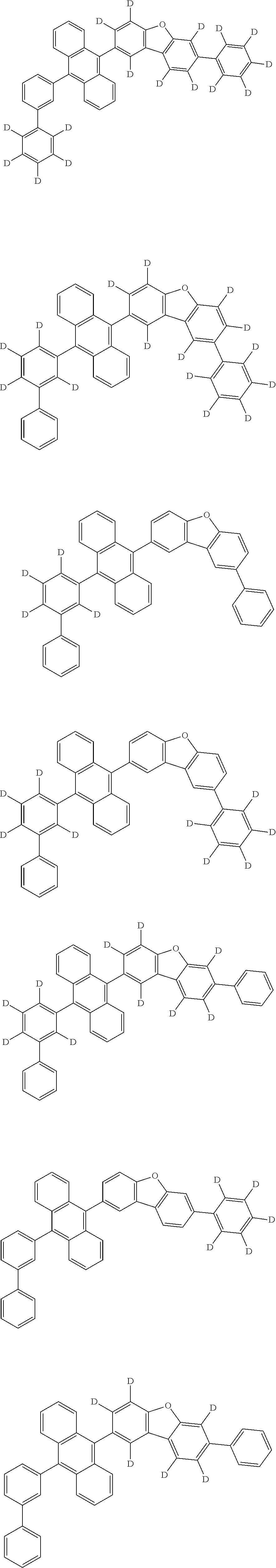

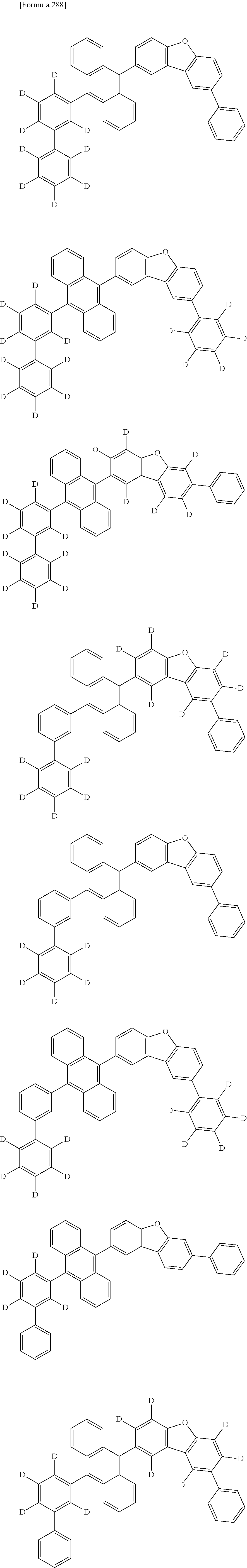

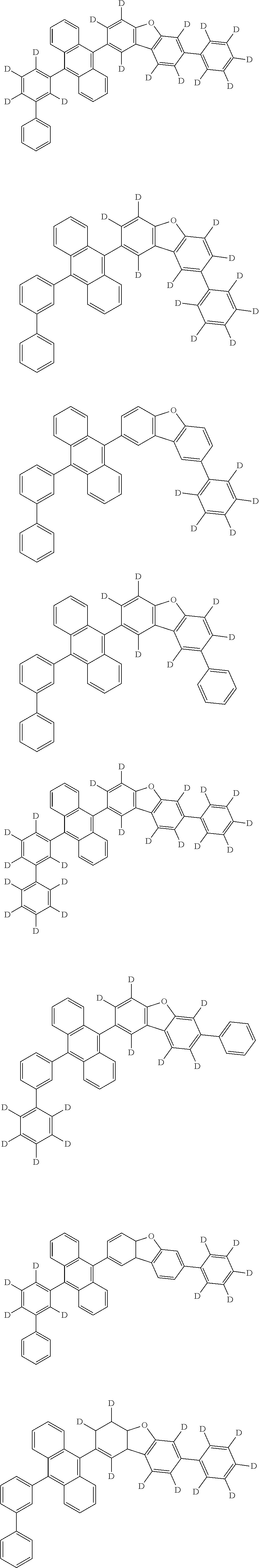

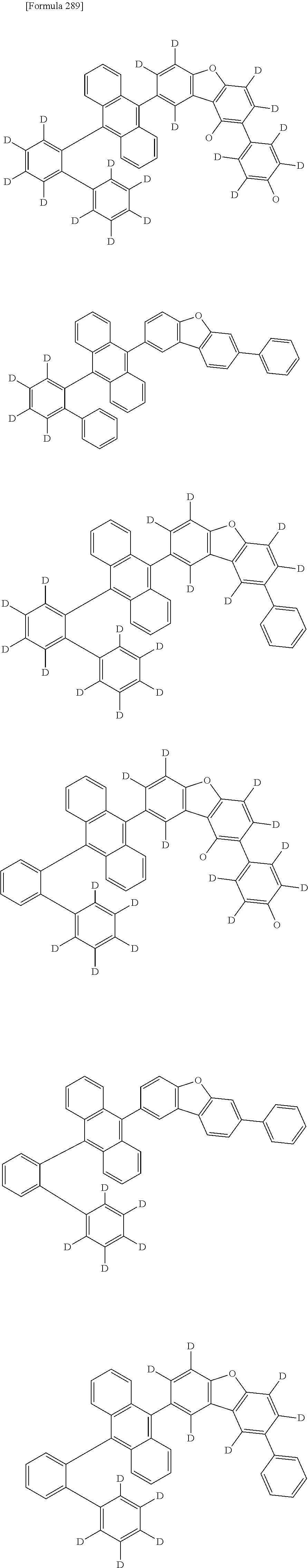

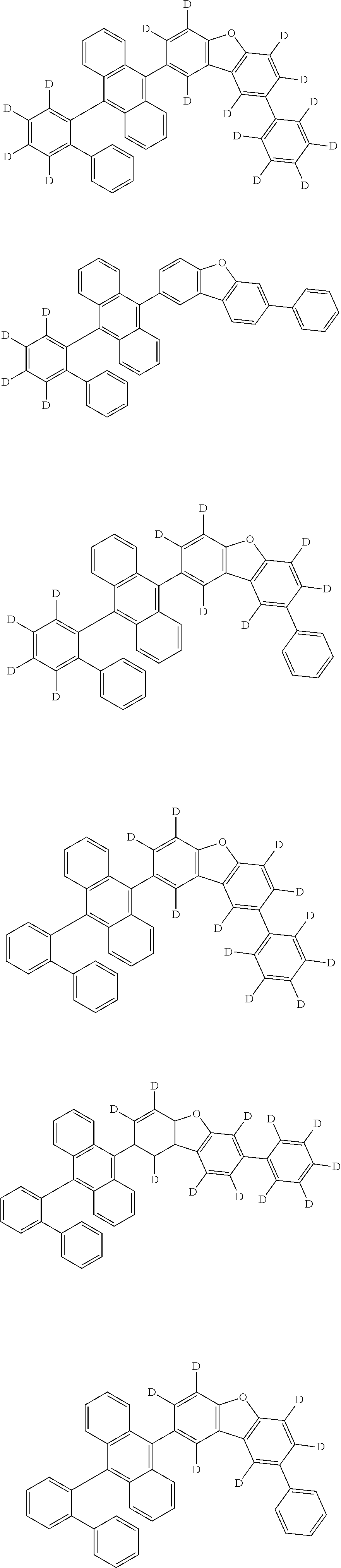

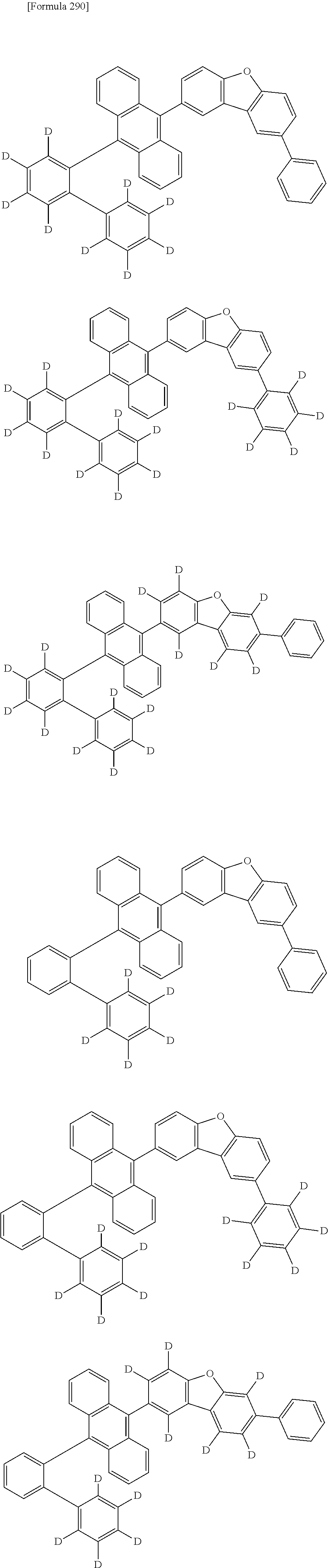

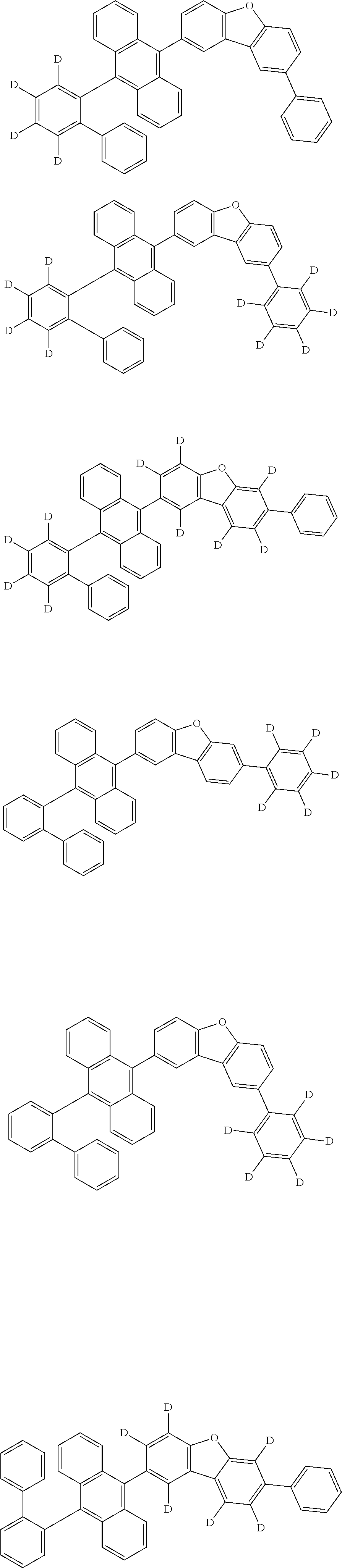

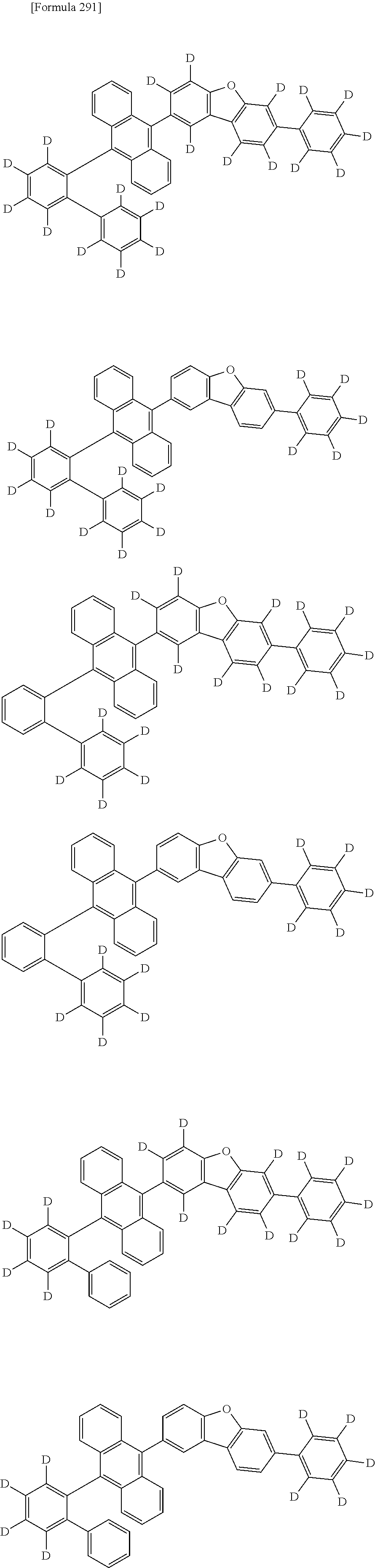

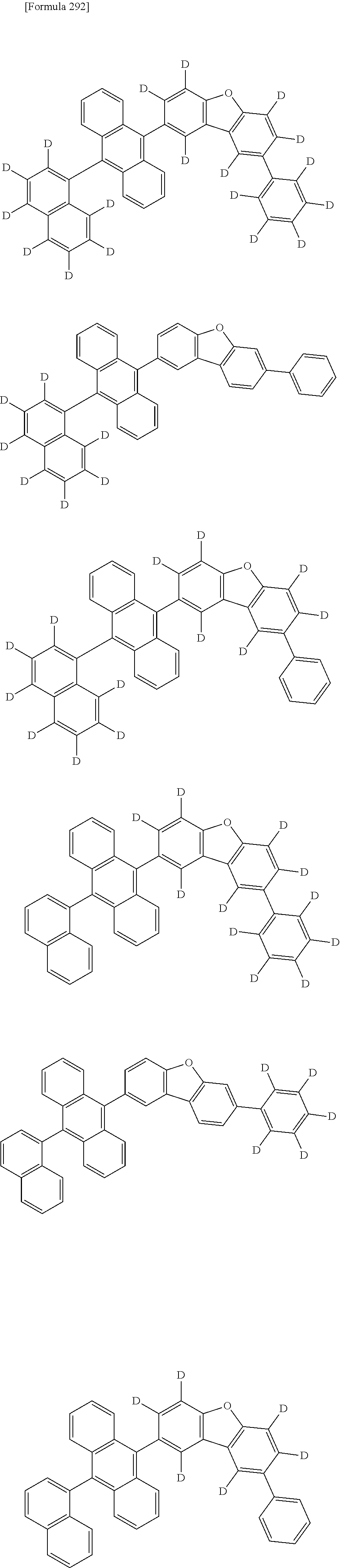

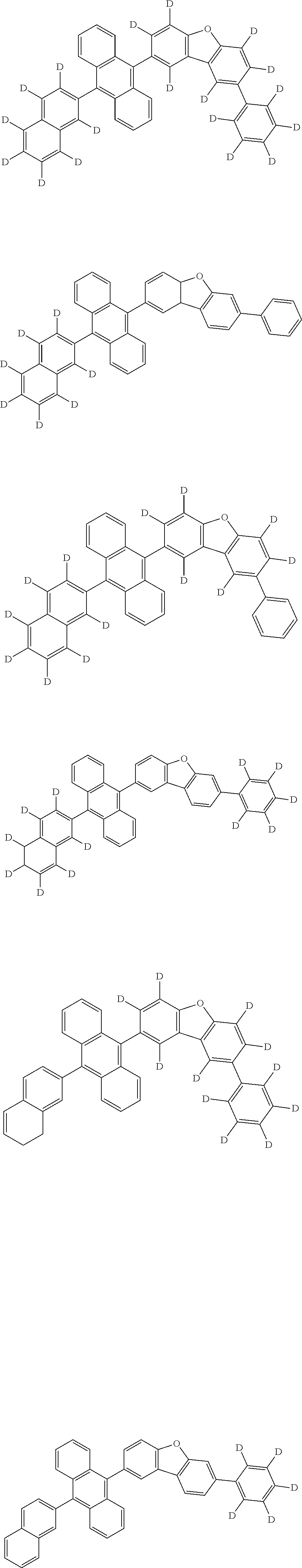

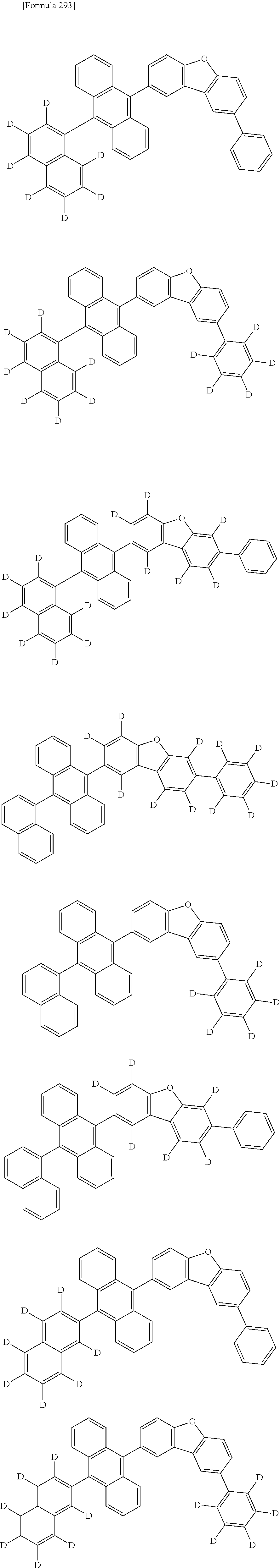

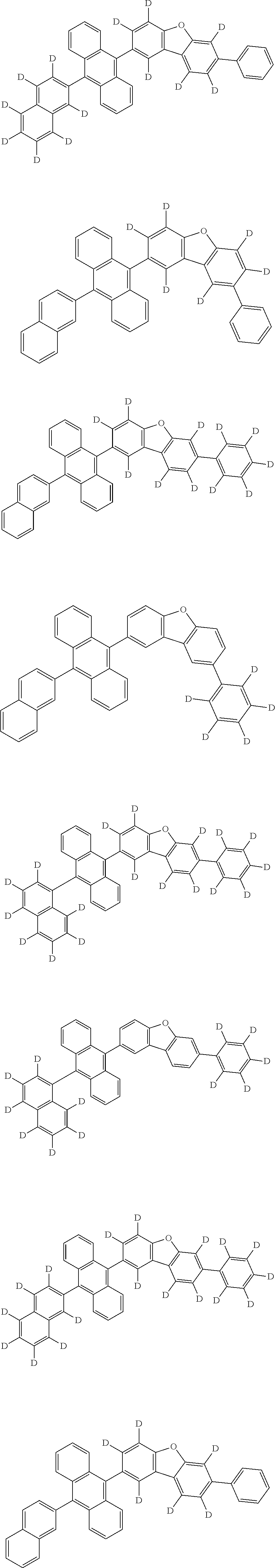

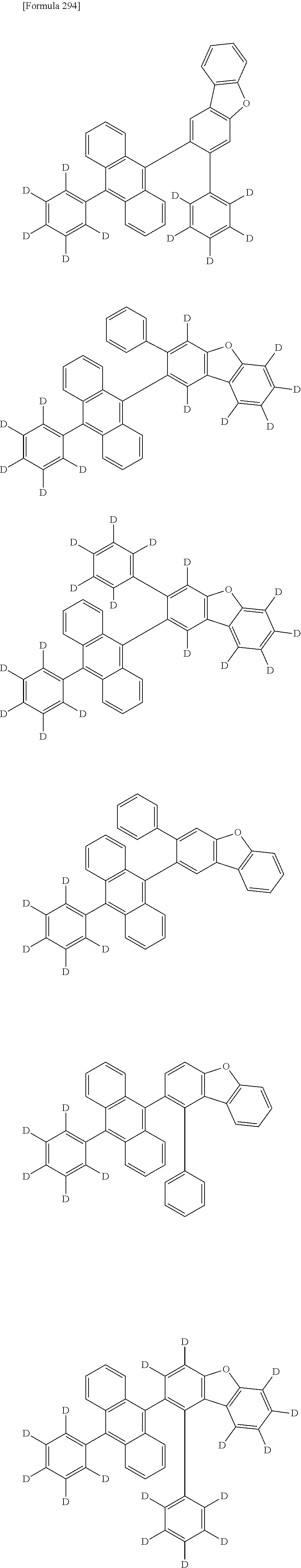

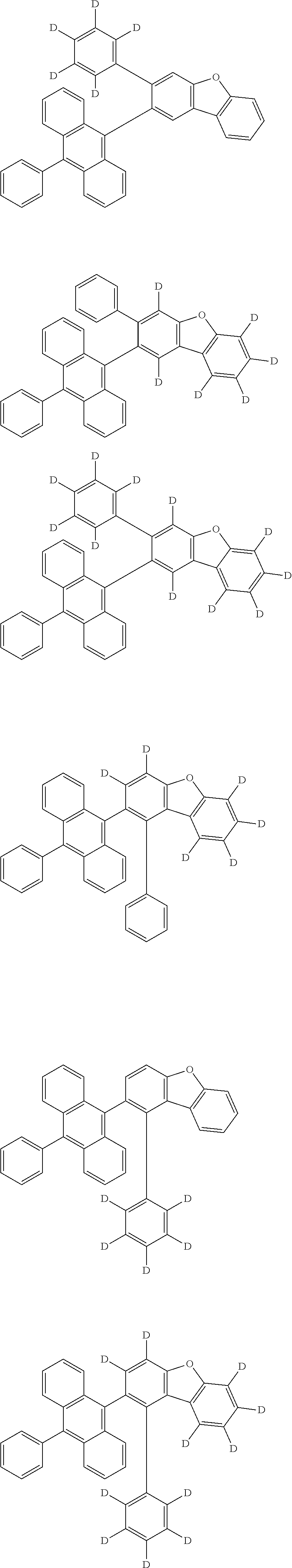

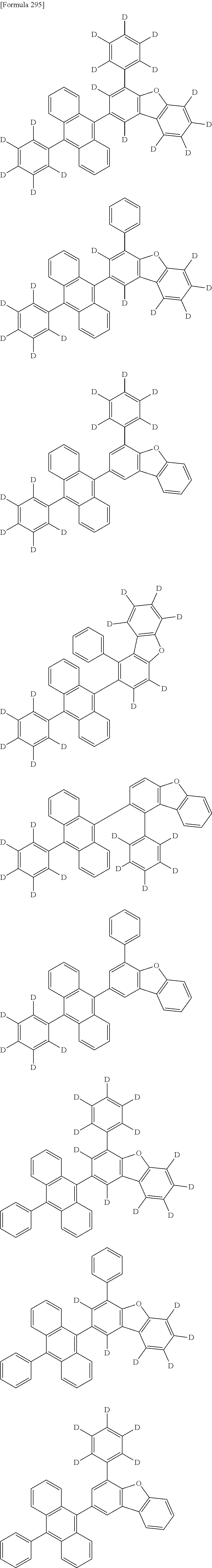

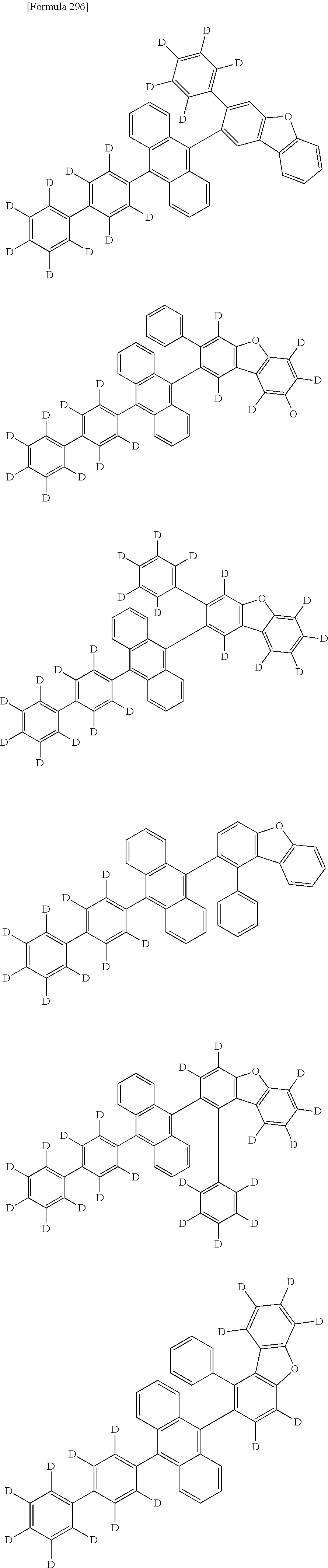

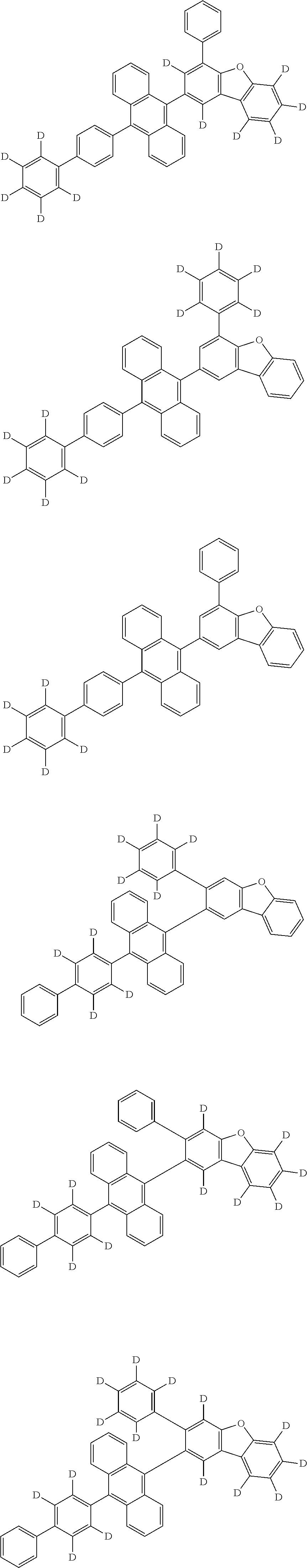

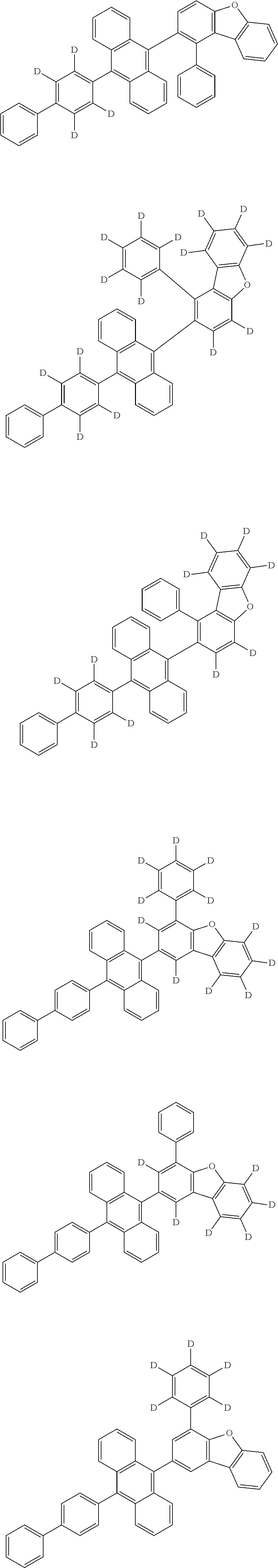

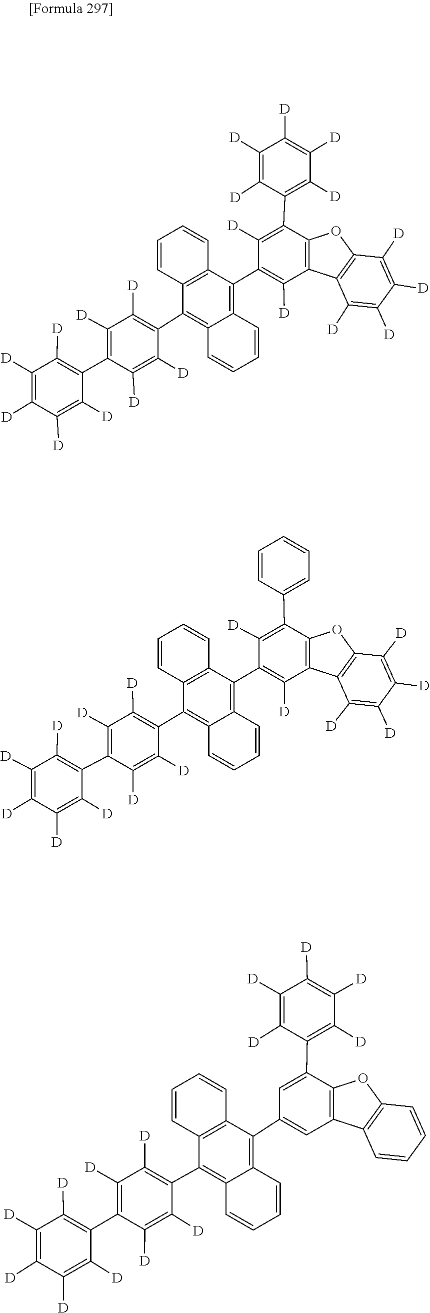 [SMILES: [2H]c1c([2H])c([2H])c(-c2c([2H])c([2H])c(-c3c4ccccc4c(-c4c([2H])c(-c5c([2H])c([2H])c([2H])c([2H])c5[2H])c5oc6c([2H])c([2H])c([2H])c([2H])c6c5c4[2H])c4ccccc34)c([2H])c2[2H])c([2H])c1[2H] ; [2H]c1c([2H])c([2H])c(-c2c([2H])c([2H])c(-c3c4ccccc4c(-c4c([2H])c(-c5ccccc5)c5oc6c([2H])c([2H])c([2H])c([2H])c6c5c4[2H])c4ccccc34)c([2H])c2[2H])c([2H])c1[2H] ; [2H]c1c([2H])c([2H])c(-c2c([2H])c([2H])c(-c3c4ccccc4c(-c4cc(-c5c([2H])c([2H])c([2H])c([2H])c5[2H])c5oc6ccccc6c5c4)c4ccccc34)c([2H])c2[2H])c([2H])c1[2H]]

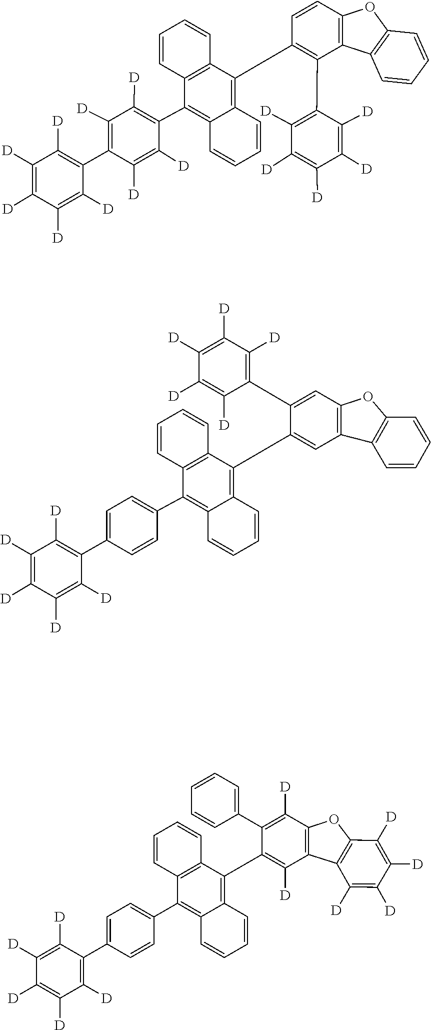 [SMILES: [2H]c1c([2H])c([2H])c(-c2c([2H])c([2H])c(-c3c4ccccc4c(-c4ccc5oc6ccccc6c5c4-c4c([2H])c([2H])c([2H])c([2H])c4[2H])c4ccccc34)c([2H])c2[2H])c([2H])c1[2H] ; [2H]c1c([2H])c([2H])c(-c2ccc(-c3c4ccccc4c(-c4cc5c(cc4-c4c([2H])c([2H])c([2H])c([2H])c4[2H])oc4ccccc45)c4ccccc34)cc2)c([2H])c1[2H] ; [2H]c1c([2H])c([2H])c(-c2ccc(-c3c4ccccc4c(-c4c(-c5ccccc5)c([2H])c5oc6c([2H])c([2H])c([2H])c([2H])c6c5c4[2H])c4ccccc34)cc2)c([2H])c1[2H]]

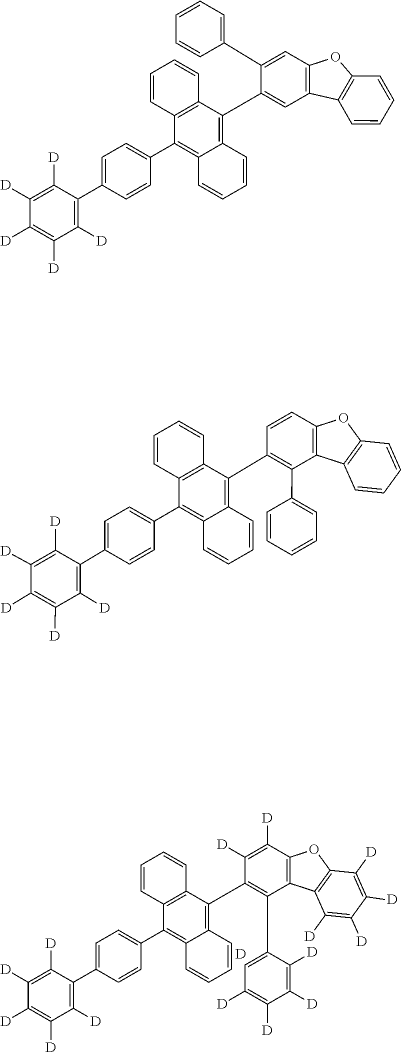

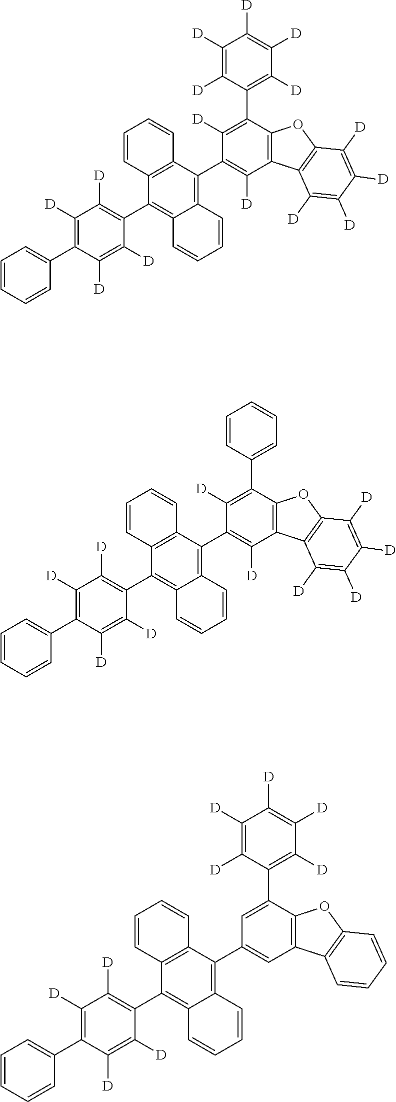 [SMILES: [2H]c1c([2H])c([2H])c(-c2c([2H])c(-c3c4ccccc4c(-c4c([2H])c([2H])c(-c5ccccc5)c([2H])c4[2H])c4ccccc34)c([2H])c3c2oc2c([2H])c([2H])c([2H])c([2H])c23)c([2H])c1[2H] ; [2H]c1c([2H])c(-c2c3ccccc3c(-c3c([2H])c(-c4ccccc4)c4oc5c([2H])c([2H])c([2H])c([2H])c5c4c3[2H])c3ccccc23)c([2H])c([2H])c1-c1ccccc1 ; [2H]c1c([2H])c([2H])c(-c2cc(-c3c4ccccc4c(-c4c([2H])c([2H])c(-c5ccccc5)c([2H])c4[2H])c4ccccc34)cc3c2oc2ccccc23)c([2H])c1[2H]]

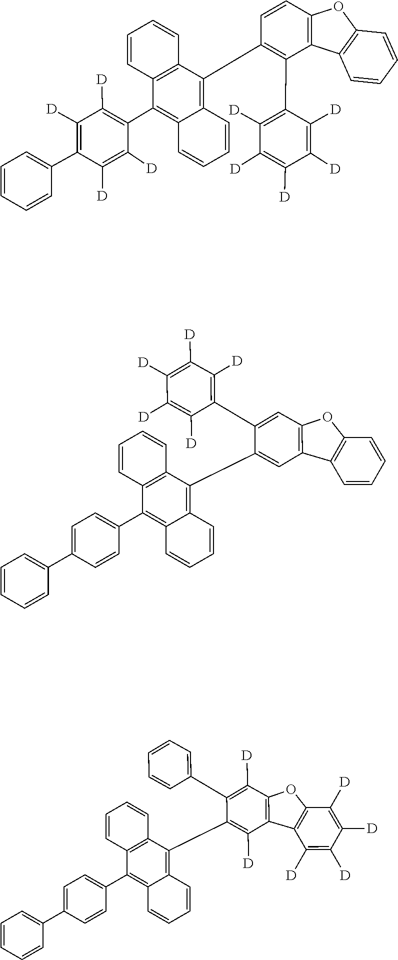 [SMILES: [2H]c1c([2H])c([2H])c(-c2c(-c3c4ccccc4c(-c4c([2H])c([2H])c(-c5ccccc5)c([2H])c4[2H])c4ccccc34)ccc3oc4ccccc4c23)c([2H])c1[2H] ; [2H]c1c([2H])c([2H])c(-c2cc3oc4ccccc4c3cc2-c2c3ccccc3c(-c3ccc(-c4ccccc4)cc3)c3ccccc23)c([2H])c1[2H] ; [2H]c1c([2H])c([2H])c2c(oc3c([2H])c(-c4ccccc4)c(-c4c5ccccc5c(-c5ccc(-c6ccccc6)cc5)c5ccccc45)c([2H])c32)c1[2H]]

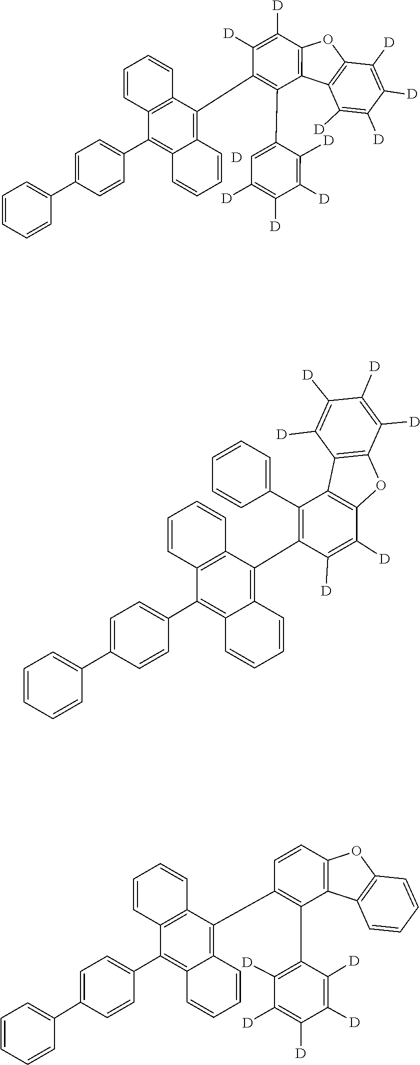

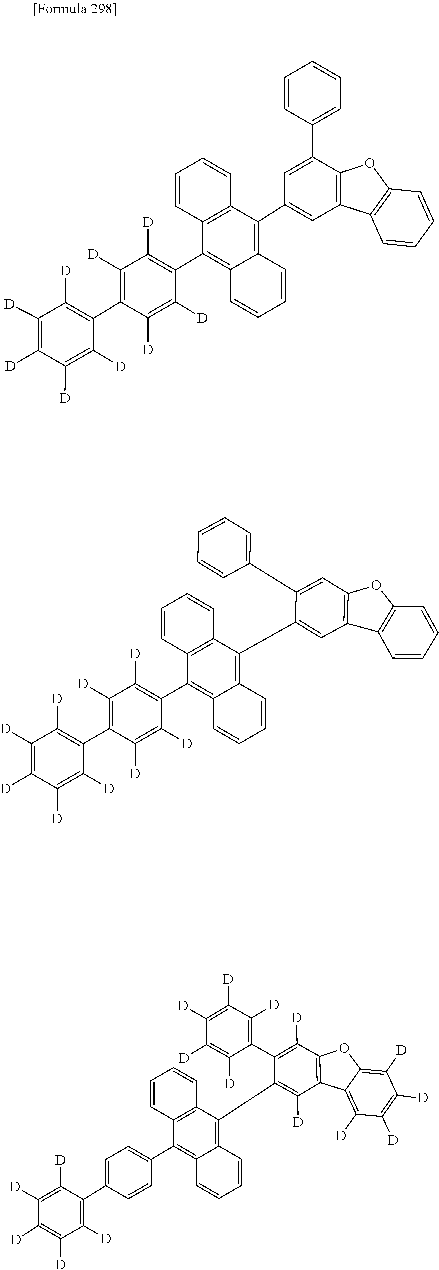 [SMILES: [2H]c1c([2H])c([2H])c(-c2c([2H])c([2H])c(-c3c4ccccc4c(-c4cc(-c5ccccc5)c5oc6ccccc6c5c4)c4ccccc34)c([2H])c2[2H])c([2H])c1[2H] ; [2H]c1c([2H])c([2H])c(-c2c([2H])c([2H])c(-c3c4ccccc4c(-c4cc5c(cc4-c4ccccc4)oc4ccccc45)c4ccccc34)c([2H])c2[2H])c([2H])c1[2H] ; [2H]c1c([2H])c([2H])c(-c2ccc(-c3c4ccccc4c(-c4c(-c5c([2H])c([2H])c([2H])c([2H])c5[2H])c([2H])c5oc6c([2H])c([2H])c([2H])c([2H])c6c5c4[2H])c4ccccc34)cc2)c([2H])c1[2H]]

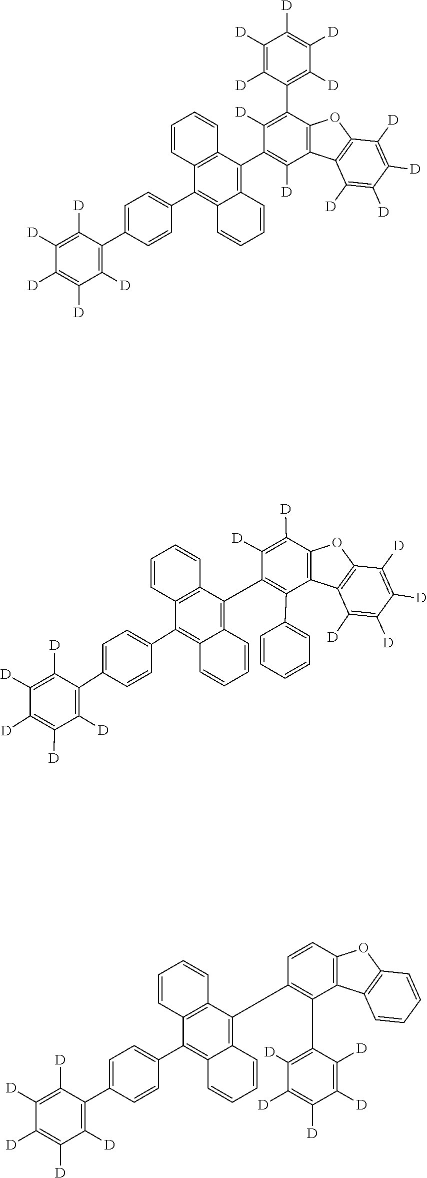 [SMILES: [2H]c1c([2H])c([2H])c(-c2ccc(-c3c4ccccc4c(-c4c([2H])c(-c5c([2H])c([2H])c([2H])c([2H])c5[2H])c5oc6c([2H])c([2H])c([2H])c([2H])c6c5c4[2H])c4ccccc34)cc2)c([2H])c1[2H] ; [2H]c1c([2H])c([2H])c(-c2ccc(-c3c4ccccc4c(-c4c([2H])c([2H])c5oc6c([2H])c([2H])c([2H])c([2H])c6c5c4-c4ccccc4)c4ccccc34)cc2)c([2H])c1[2H] ; [2H]c1c([2H])c([2H])c(-c2ccc(-c3c4ccccc4c(-c4ccc5oc6ccccc6c5c4-c4c([2H])c([2H])c([2H])c([2H])c4[2H])c4ccccc34)cc2)c([2H])c1[2H]]

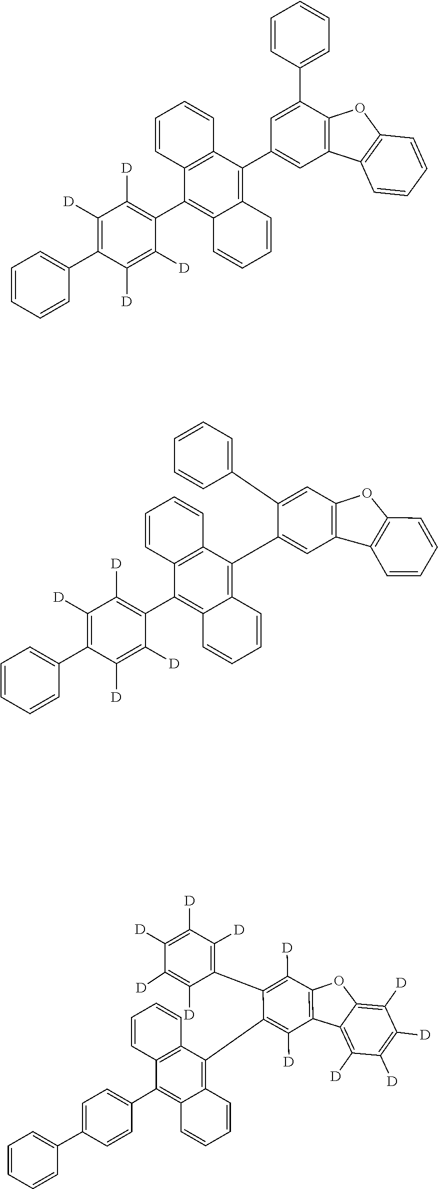 [SMILES: [2H]c1c([2H])c(-c2c3ccccc3c(-c3cc(-c4ccccc4)c4oc5ccccc5c4c3)c3ccccc23)c([2H])c([2H])c1-c1ccccc1 ; [2H]c1c([2H])c(-c2c3ccccc3c(-c3cc4c(cc3-c3ccccc3)oc3ccccc34)c3ccccc23)c([2H])c([2H])c1-c1ccccc1 ; [2H]c1c([2H])c([2H])c(-c2c(-c3c4ccccc4c(-c4ccc(-c5ccccc5)cc4)c4ccccc34)c([2H])c3c(oc4c([2H])c([2H])c([2H])c([2H])c43)c2[2H])c([2H])c1[2H]]

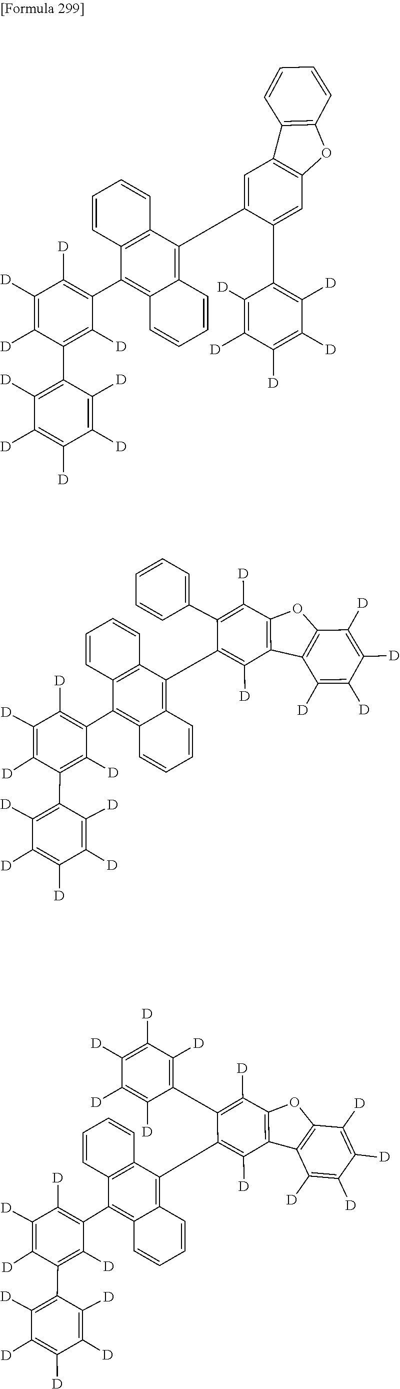 [SMILES: [2H]c1c([2H])c([2H])c(-c2cc3oc4ccccc4c3cc2-c2c3ccccc3c(-c3c([2H])c([2H])c([2H])c(-c4c([2H])c([2H])c([2H])c([2H])c4[2H])c3[2H])c3ccccc23)c([2H])c1[2H] ; [2H]c1c([2H])c([2H])c(-c2c([2H])c([2H])c([2H])c(-c3c4ccccc4c(-c4c(-c5ccccc5)c([2H])c5oc6c([2H])c([2H])c([2H])c([2H])c6c5c4[2H])c4ccccc34)c2[2H])c([2H])c1[2H] ; [2H]c1c([2H])c([2H])c(-c2c([2H])c([2H])c([2H])c(-c3c4ccccc4c(-c4c(-c5c([2H])c([2H])c([2H])c([2H])c5[2H])c([2H])c5oc6c([2H])c([2H])c([2H])c([2H])c6c5c4[2H])c4ccccc34)c2[2H])c([2H])c1[2H]]

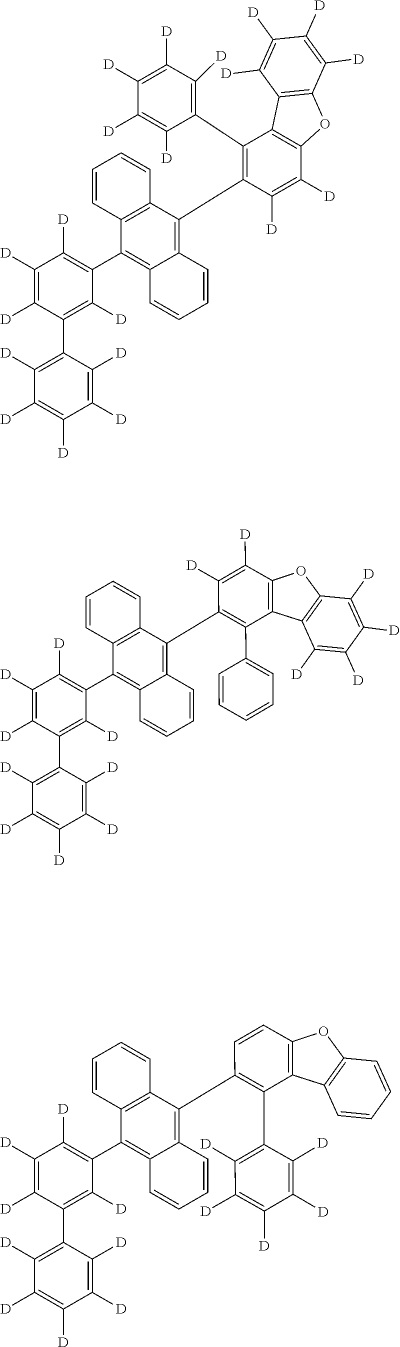 [SMILES: [2H]c1c([2H])c([2H])c(-c2c([2H])c([2H])c([2H])c(-c3c4ccccc4c(-c4c([2H])c([2H])c5oc6c([2H])c([2H])c([2H])c([2H])c6c5c4-c4c([2H])c([2H])c([2H])c([2H])c4[2H])c4ccccc34)c2[2H])c([2H])c1[2H] ; [2H]c1c([2H])c([2H])c(-c2c([2H])c([2H])c([2H])c(-c3c4ccccc4c(-c4c([2H])c([2H])c5oc6c([2H])c([2H])c([2H])c([2H])c6c5c4-c4ccccc4)c4ccccc34)c2[2H])c([2H])c1[2H] ; [2H]c1c([2H])c([2H])c(-c2c([2H])c([2H])c([2H])c(-c3c4ccccc4c(-c4ccc5oc6ccccc6c5c4-c4c([2H])c([2H])c([2H])c([2H])c4[2H])c4ccccc34)c2[2H])c([2H])c1[2H]]

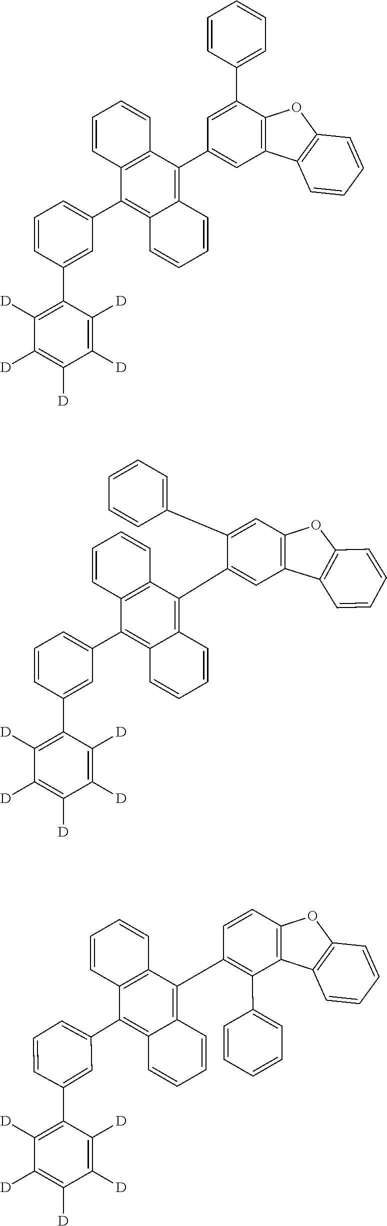 [SMILES: [2H]c1c([2H])c([2H])c(-c2cccc(-c3c4ccccc4c(-c4cc(-c5ccccc5)c5oc6ccccc6c5c4)c4ccccc34)c2)c([2H])c1[2H] ; [2H]c1c([2H])c([2H])c(-c2cccc(-c3c4ccccc4c(-c4cc5c(cc4-c4ccccc4)oc4ccccc45)c4ccccc34)c2)c([2H])c1[2H] ; [2H]c1c([2H])c([2H])c(-c2cccc(-c3c4ccccc4c(-c4ccc5oc6ccccc6c5c4-c4ccccc4)c4ccccc34)c2)c([2H])c1[2H]]

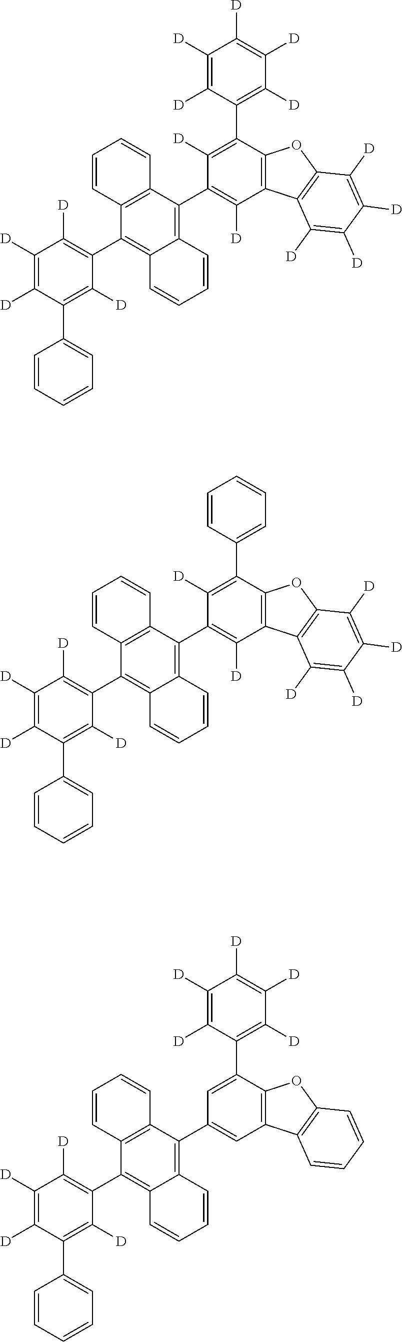 [SMILES: [2H]c1c([2H])c([2H])c(-c2c([2H])c(-c3c4ccccc4c(-c4c([2H])c([2H])c([2H])c(-c5ccccc5)c4[2H])c4ccccc34)c([2H])c3c2oc2c([2H])c([2H])c([2H])c([2H])c23)c([2H])c1[2H] ; [2H]c1c([2H])c(-c2ccccc2)c([2H])c(-c2c3ccccc3c(-c3c([2H])c(-c4ccccc4)c4oc5c([2H])c([2H])c([2H])c([2H])c5c4c3[2H])c3ccccc23)c1[2H] ; [2H]c1c([2H])c([2H])c(-c2cc(-c3c4ccccc4c(-c4c([2H])c([2H])c([2H])c(-c5ccccc5)c4[2H])c4ccccc34)cc3c2oc2ccccc23)c([2H])c1[2H]]

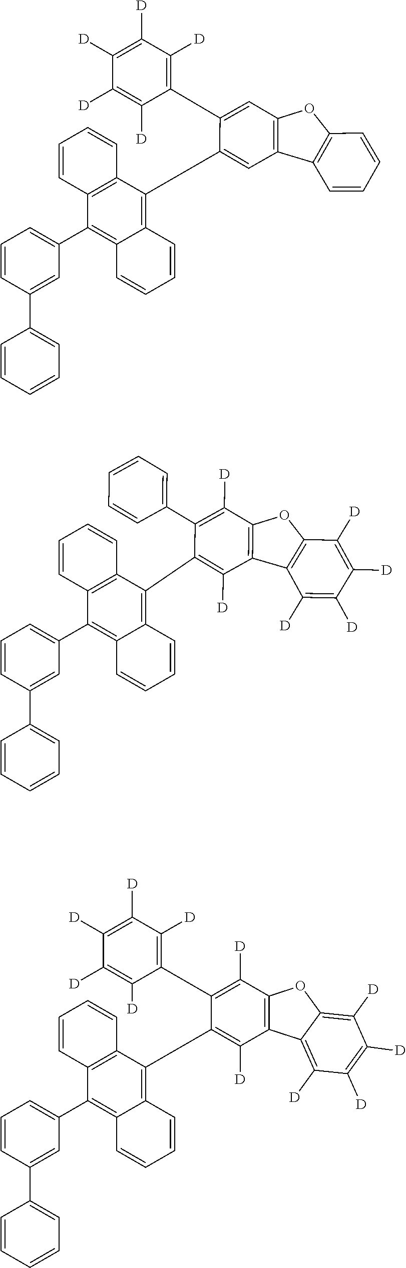 [SMILES: [2H]c1c([2H])c([2H])c(-c2cc3oc4ccccc4c3cc2-c2c3ccccc3c(-c3cccc(-c4ccccc4)c3)c3ccccc23)c([2H])c1[2H] ; [2H]c1c([2H])c([2H])c2c(oc3c([2H])c(-c4ccccc4)c(-c4c5ccccc5c(-c5cccc(-c6ccccc6)c5)c5ccccc45)c([2H])c32)c1[2H] ; [2H]c1c([2H])c([2H])c(-c2c(-c3c4ccccc4c(-c4cccc(-c5ccccc5)c4)c4ccccc34)c([2H])c3c(oc4c([2H])c([2H])c([2H])c([2H])c43)c2[2H])c([2H])c1[2H]]

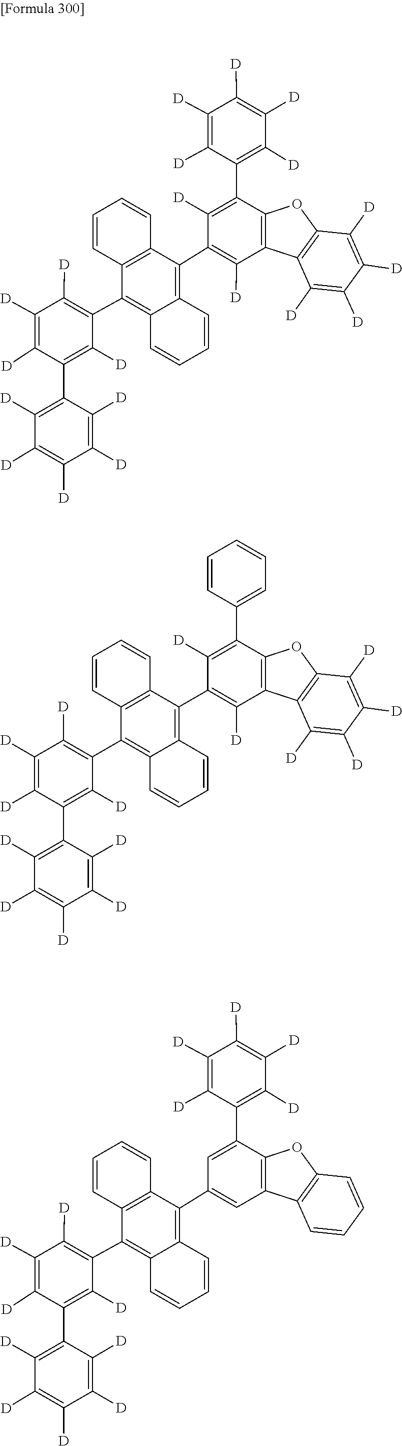 [SMILES: [2H]c1c([2H])c([2H])c(-c2c([2H])c([2H])c([2H])c(-c3c4ccccc4c(-c4c([2H])c(-c5c([2H])c([2H])c([2H])c([2H])c5[2H])c5oc6c([2H])c([2H])c([2H])c([2H])c6c5c4[2H])c4ccccc34)c2[2H])c([2H])c1[2H] ; [2H]c1c([2H])c([2H])c(-c2c([2H])c([2H])c([2H])c(-c3c4ccccc4c(-c4c([2H])c(-c5ccccc5)c5oc6c([2H])c([2H])c([2H])c([2H])c6c5c4[2H])c4ccccc34)c2[2H])c([2H])c1[2H] ; [2H]c1c([2H])c([2H])c(-c2c([2H])c([2H])c([2H])c(-c3c4ccccc4c(-c4cc(-c5c([2H])c([2H])c([2H])c([2H])c5[2H])c5oc6ccccc6c5c4)c4ccccc34)c2[2H])c([2H])c1[2H]]

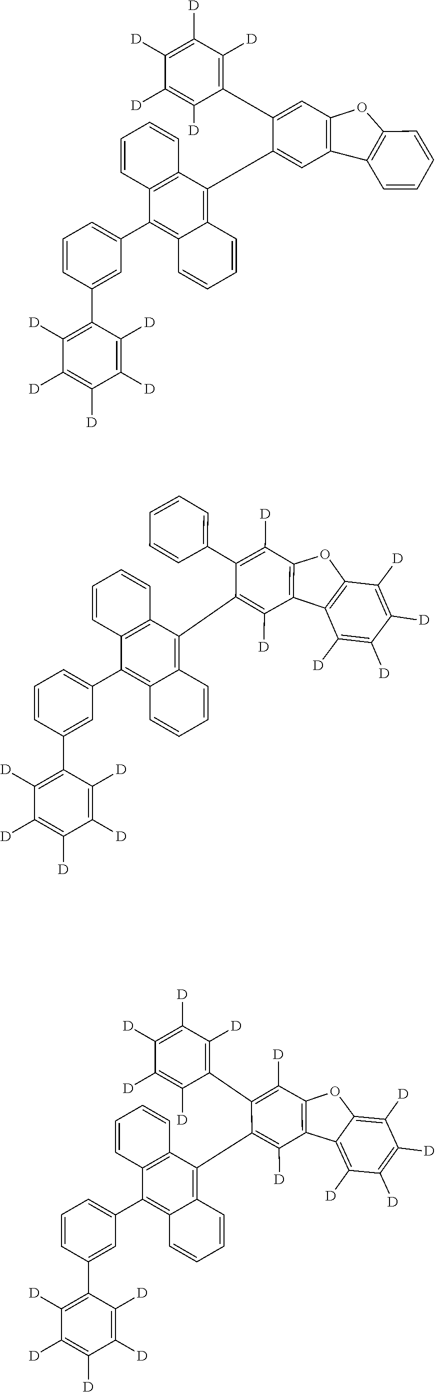 [SMILES: [2H]c1c([2H])c([2H])c(-c2cccc(-c3c4ccccc4c(-c4cc5c(cc4-c4c([2H])c([2H])c([2H])c([2H])c4[2H])oc4ccccc45)c4ccccc34)c2)c([2H])c1[2H] ; [2H]c1c([2H])c([2H])c(-c2cccc(-c3c4ccccc4c(-c4c(-c5ccccc5)c([2H])c5oc6c([2H])c([2H])c([2H])c([2H])c6c5c4[2H])c4ccccc34)c2)c([2H])c1[2H] ; [2H]c1c([2H])c([2H])c(-c2cccc(-c3c4ccccc4c(-c4c(-c5c([2H])c([2H])c([2H])c([2H])c5[2H])c([2H])c5oc6c([2H])c([2H])c([2H])c([2H])c6c5c4[2H])c4ccccc34)c2)c([2H])c1[2H]]

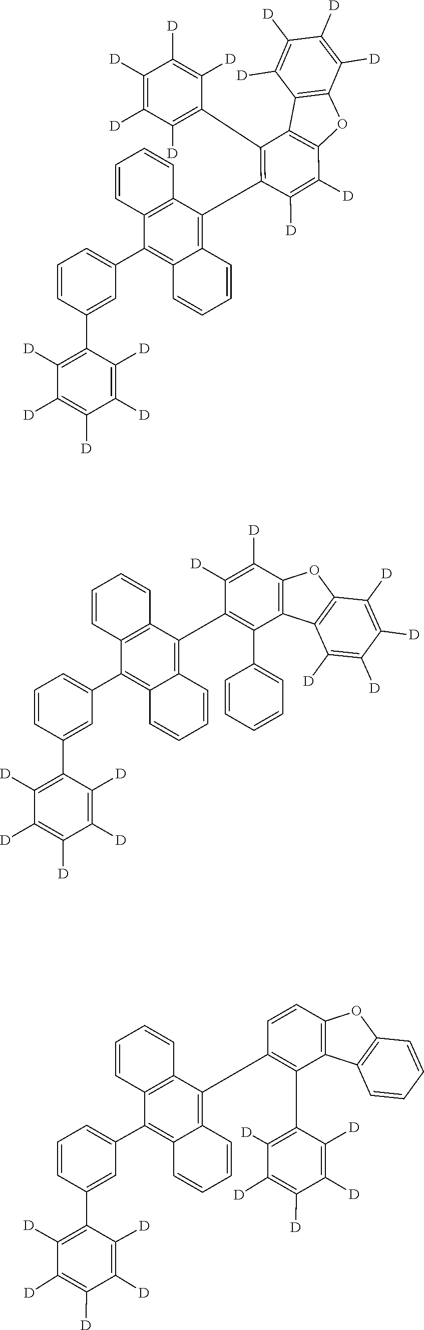 [SMILES: [2H]c1c([2H])c([2H])c(-c2cccc(-c3c4ccccc4c(-c4c([2H])c([2H])c5oc6c([2H])c([2H])c([2H])c([2H])c6c5c4-c4c([2H])c([2H])c([2H])c([2H])c4[2H])c4ccccc34)c2)c([2H])c1[2H] ; [2H]c1c([2H])c([2H])c(-c2cccc(-c3c4ccccc4c(-c4c([2H])c([2H])c5oc6c([2H])c([2H])c([2H])c([2H])c6c5c4-c4ccccc4)c4ccccc34)c2)c([2H])c1[2H] ; [2H]c1c([2H])c([2H])c(-c2cccc(-c3c4ccccc4c(-c4ccc5oc6ccccc6c5c4-c4c([2H])c([2H])c([2H])c([2H])c4[2H])c4ccccc34)c2)c([2H])c1[2H]]

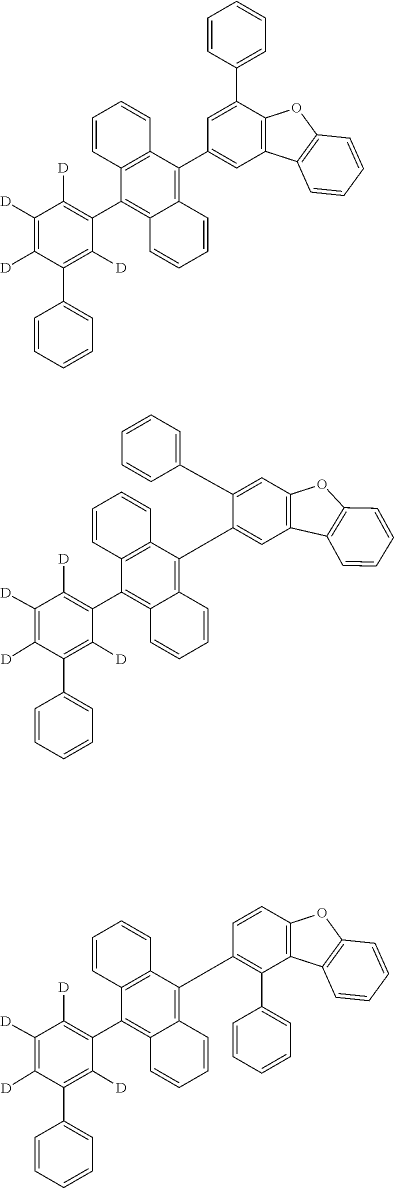 [SMILES: [2H]c1c([2H])c(-c2ccccc2)c([2H])c(-c2c3ccccc3c(-c3cc(-c4ccccc4)c4oc5ccccc5c4c3)c3ccccc23)c1[2H] ; [2H]c1c([2H])c(-c2ccccc2)c([2H])c(-c2c3ccccc3c(-c3cc4c(cc3-c3ccccc3)oc3ccccc34)c3ccccc23)c1[2H] ; [2H]c1c([2H])c(-c2ccccc2)c([2H])c(-c2c3ccccc3c(-c3ccc4oc5ccccc5c4c3-c3ccccc3)c3ccccc23)c1[2H]]

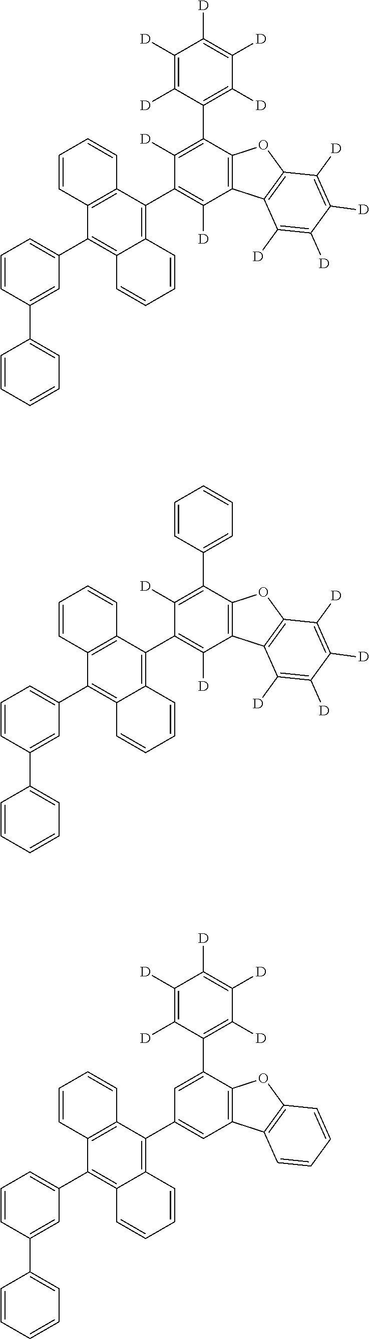 [SMILES: [2H]c1c([2H])c([2H])c(-c2c([2H])c(-c3c4ccccc4c(-c4cccc(-c5ccccc5)c4)c4ccccc34)c([2H])c3c2oc2c([2H])c([2H])c([2H])c([2H])c23)c([2H])c1[2H] ; [2H]c1c([2H])c([2H])c2c(oc3c(-c4ccccc4)c([2H])c(-c4c5ccccc5c(-c5cccc(-c6ccccc6)c5)c5ccccc45)c([2H])c32)c1[2H] ; [2H]c1c([2H])c([2H])c(-c2cc(-c3c4ccccc4c(-c4cccc(-c5ccccc5)c4)c4ccccc34)cc3c2oc2ccccc23)c([2H])c1[2H]]

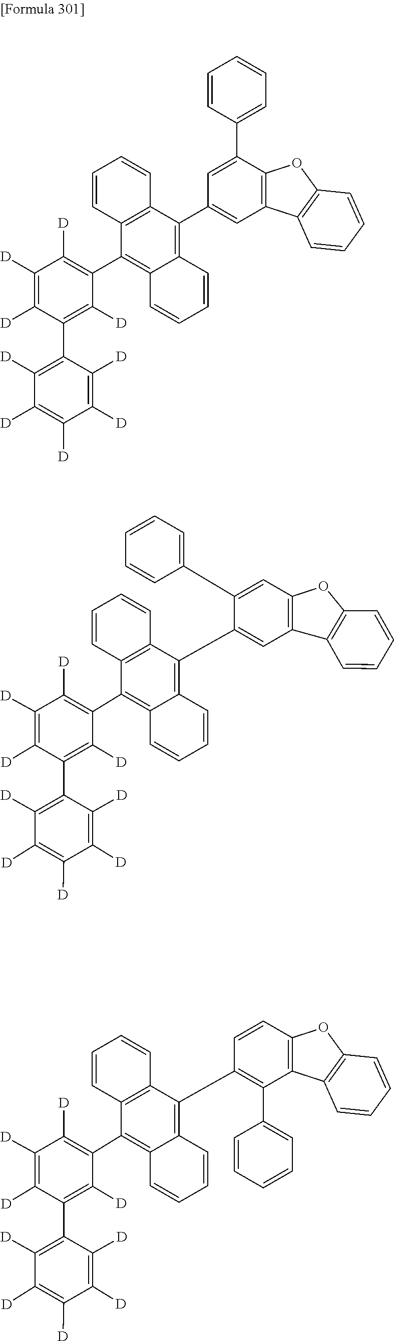 [SMILES: [2H]c1c([2H])c([2H])c(-c2c([2H])c([2H])c([2H])c(-c3c4ccccc4c(-c4cc(-c5ccccc5)c5oc6ccccc6c5c4)c4ccccc34)c2[2H])c([2H])c1[2H] ; [2H]c1c([2H])c([2H])c(-c2c([2H])c([2H])c([2H])c(-c3c4ccccc4c(-c4cc5c(cc4-c4ccccc4)oc4ccccc45)c4ccccc34)c2[2H])c([2H])c1[2H] ; [2H]c1c([2H])c([2H])c(-c2c([2H])c([2H])c([2H])c(-c3c4ccccc4c(-c4ccc5oc6ccccc6c5c4-c4ccccc4)c4ccccc34)c2[2H])c([2H])c1[2H]]

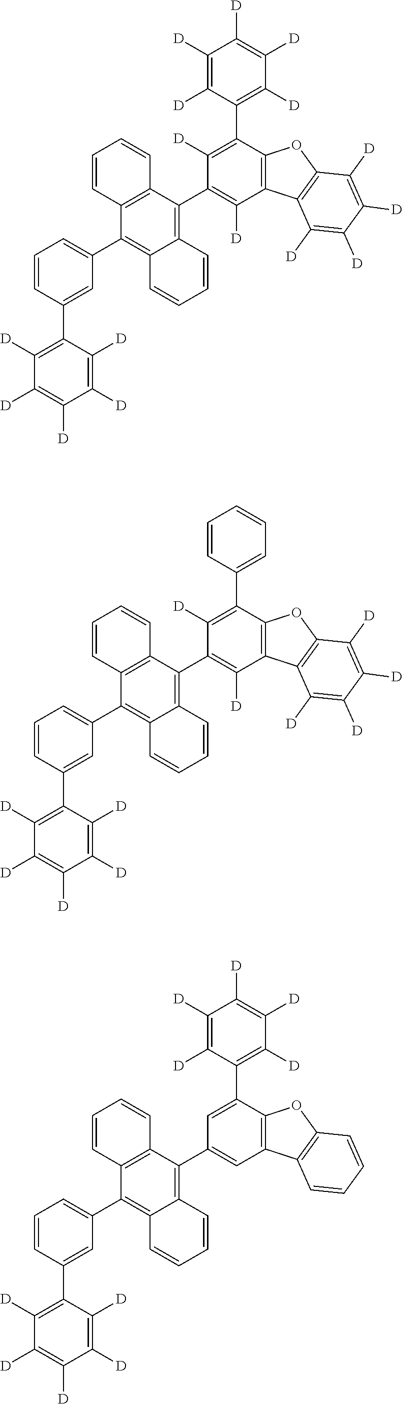 [SMILES: [2H]c1c([2H])c([2H])c(-c2cccc(-c3c4ccccc4c(-c4c([2H])c(-c5c([2H])c([2H])c([2H])c([2H])c5[2H])c5oc6c([2H])c([2H])c([2H])c([2H])c6c5c4[2H])c4ccccc34)c2)c([2H])c1[2H] ; [2H]c1c([2H])c([2H])c(-c2cccc(-c3c4ccccc4c(-c4c([2H])c(-c5ccccc5)c5oc6c([2H])c([2H])c([2H])c([2H])c6c5c4[2H])c4ccccc34)c2)c([2H])c1[2H] ; [2H]c1c([2H])c([2H])c(-c2cccc(-c3c4ccccc4c(-c4cc(-c5c([2H])c([2H])c([2H])c([2H])c5[2H])c5oc6ccccc6c5c4)c4ccccc34)c2)c([2H])c1[2H]]

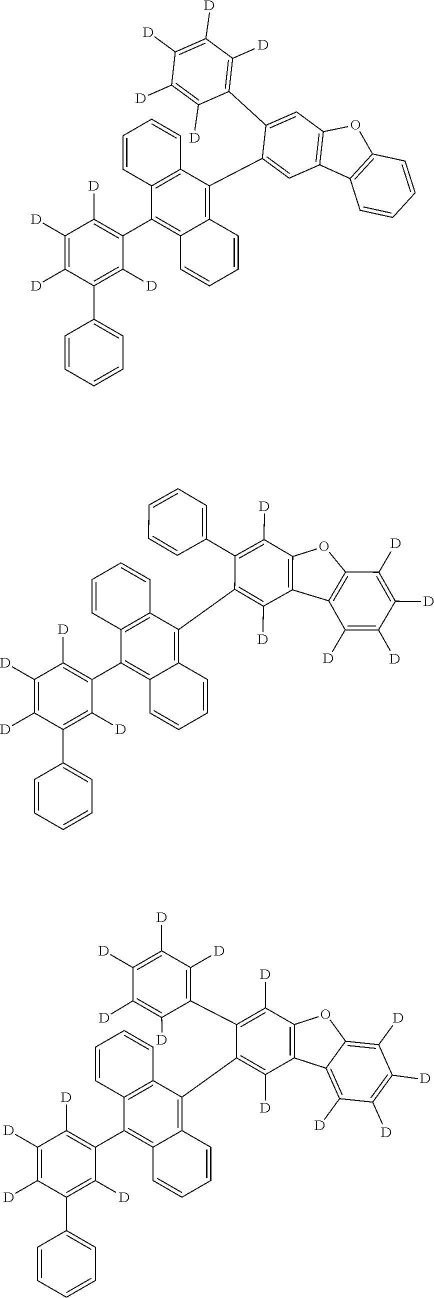 [SMILES: [2H]c1c([2H])c([2H])c(-c2cc3oc4ccccc4c3cc2-c2c3ccccc3c(-c3c([2H])c([2H])c([2H])c(-c4ccccc4)c3[2H])c3ccccc23)c([2H])c1[2H] ; [2H]c1c([2H])c(-c2ccccc2)c([2H])c(-c2c3ccccc3c(-c3c(-c4ccccc4)c([2H])c4oc5c([2H])c([2H])c([2H])c([2H])c5c4c3[2H])c3ccccc23)c1[2H] ; [2H]c1c([2H])c([2H])c(-c2c(-c3c4ccccc4c(-c4c([2H])c([2H])c([2H])c(-c5ccccc5)c4[2H])c4ccccc34)c([2H])c3c(oc4c([2H])c([2H])c([2H])c([2H])c43)c2[2H])c([2H])c1[2H]]

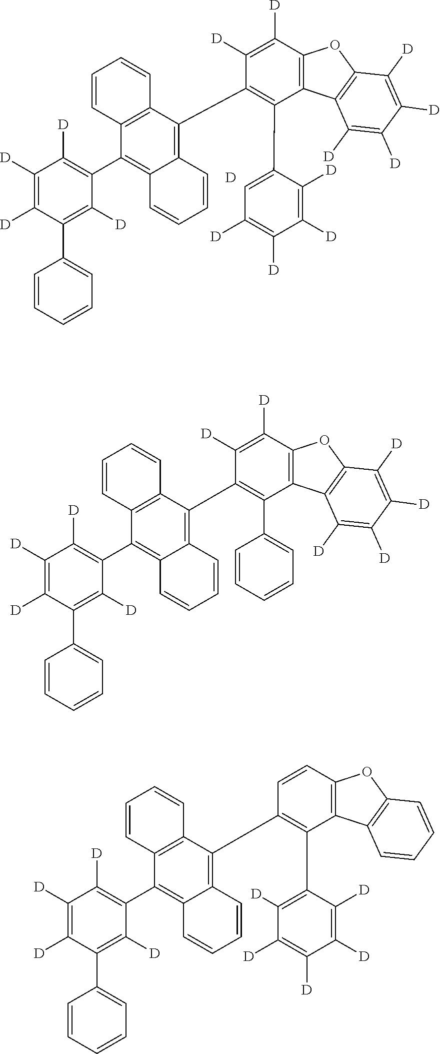

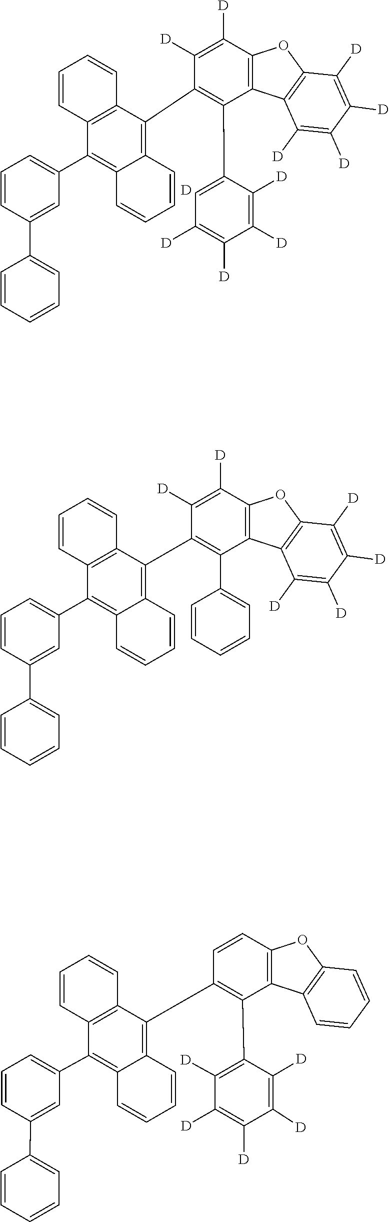

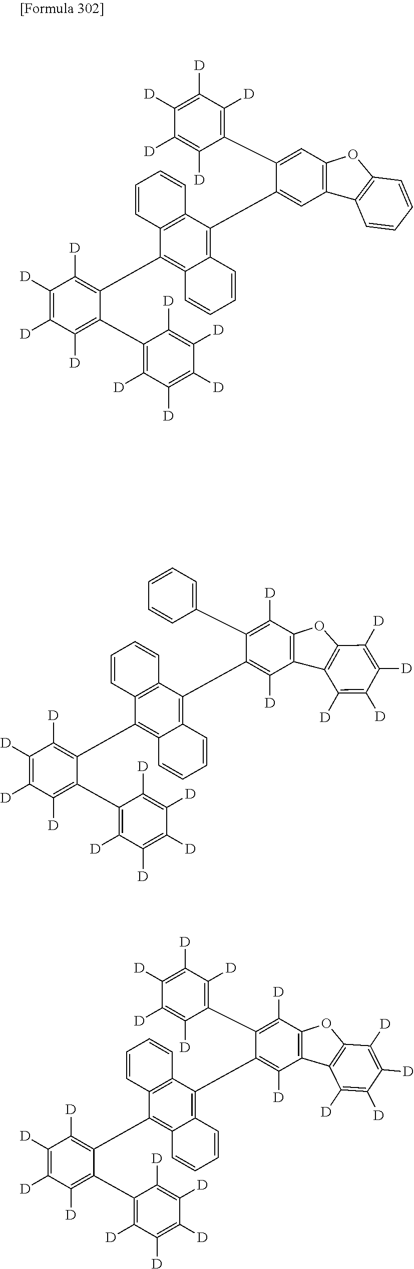 [SMILES: [2H]c1c([2H])c([2H])c(-c2cc3oc4ccccc4c3cc2-c2c3ccccc3c(-c3c([2H])c([2H])c([2H])c([2H])c3-c3c([2H])c([2H])c([2H])c([2H])c3[2H])c3ccccc23)c([2H])c1[2H] ; [2H]c1c([2H])c([2H])c(-c2c([2H])c([2H])c([2H])c([2H])c2-c2c3ccccc3c(-c3c(-c4ccccc4)c([2H])c4oc5c([2H])c([2H])c([2H])c([2H])c5c4c3[2H])c3ccccc23)c([2H])c1[2H] ; [2H]c1c([2H])c([2H])c(-c2c([2H])c([2H])c([2H])c([2H])c2-c2c3ccccc3c(-c3c(-c4c([2H])c([2H])c([2H])c([2H])c4[2H])c([2H])c4oc5c([2H])c([2H])c([2H])c([2H])c5c4c3[2H])c3ccccc23)c([2H])c1[2H]]

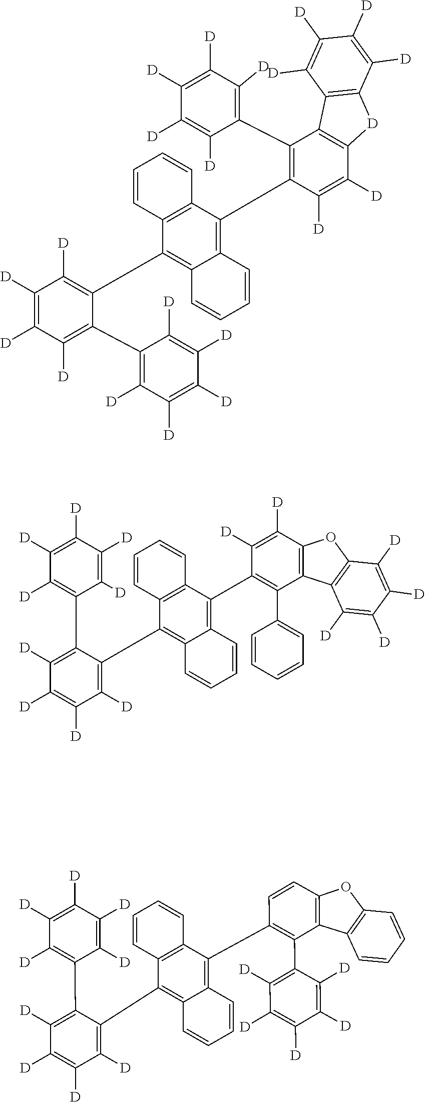

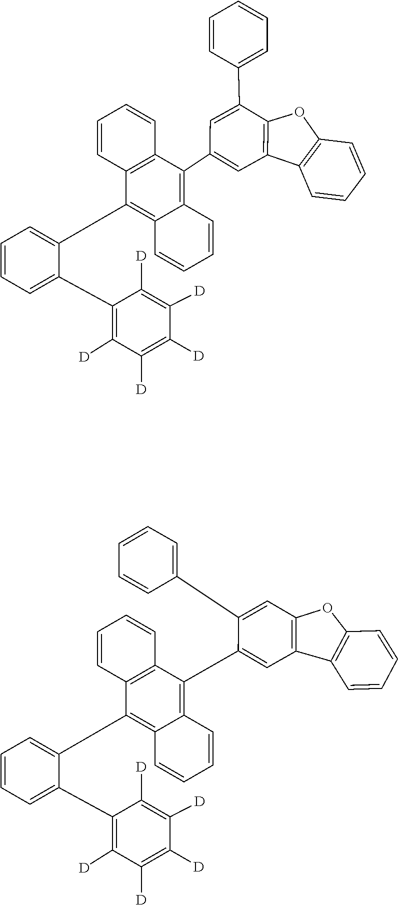 [SMILES: [2H]c1c([2H])c([2H])c(-c2ccccc2-c2c3ccccc3c(-c3cc(-c4ccccc4)c4oc5ccccc5c4c3)c3ccccc23)c([2H])c1[2H] ; [2H]c1c([2H])c([2H])c(-c2ccccc2-c2c3ccccc3c(-c3cc4c(cc3-c3ccccc3)oc3ccccc34)c3ccccc23)c([2H])c1[2H]]

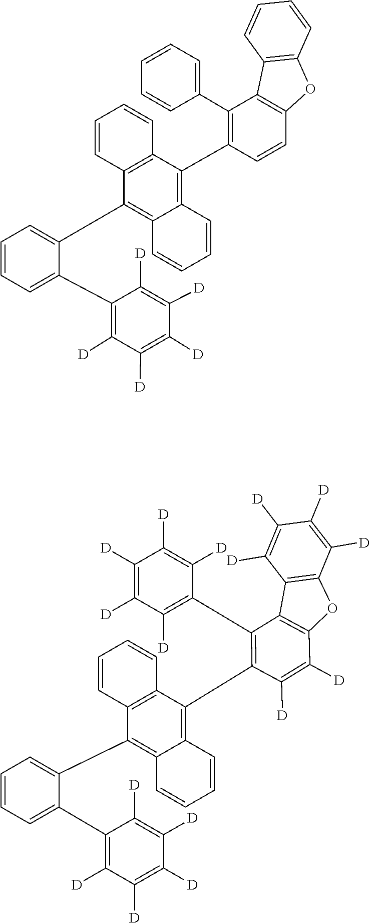 [SMILES: [2H]c1c([2H])c([2H])c(-c2ccccc2-c2c3ccccc3c(-c3ccc4oc5ccccc5c4c3-c3ccccc3)c3ccccc23)c([2H])c1[2H] ; [2H]c1c([2H])c([2H])c(-c2ccccc2-c2c3ccccc3c(-c3c([2H])c([2H])c4oc5c([2H])c([2H])c([2H])c([2H])c5c4c3-c3c([2H])c([2H])c([2H])c([2H])c3[2H])c3ccccc23)c([2H])c1[2H]]

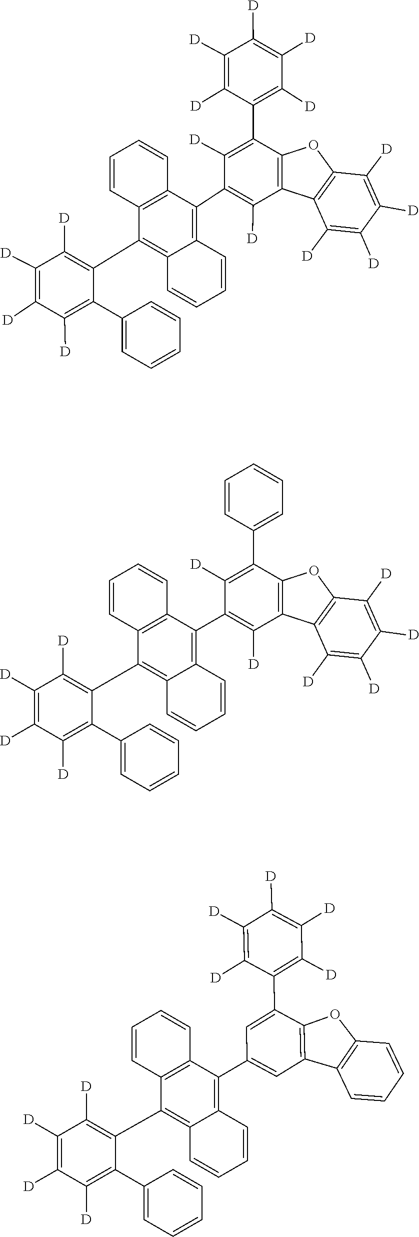 [SMILES: [2H]c1c([2H])c([2H])c(-c2c([2H])c(-c3c4ccccc4c(-c4c([2H])c([2H])c([2H])c([2H])c4-c4ccccc4)c4ccccc34)c([2H])c3c2oc2c([2H])c([2H])c([2H])c([2H])c23)c([2H])c1[2H] ; [2H]c1c([2H])c([2H])c(-c2c3ccccc3c(-c3c([2H])c(-c4ccccc4)c4oc5c([2H])c([2H])c([2H])c([2H])c5c4c3[2H])c3ccccc23)c(-c2ccccc2)c1[2H] ; [2H]c1c([2H])c([2H])c(-c2cc(-c3c4ccccc4c(-c4c([2H])c([2H])c([2H])c([2H])c4-c4ccccc4)c4ccccc34)cc3c2oc2ccccc23)c([2H])c1[2H]]

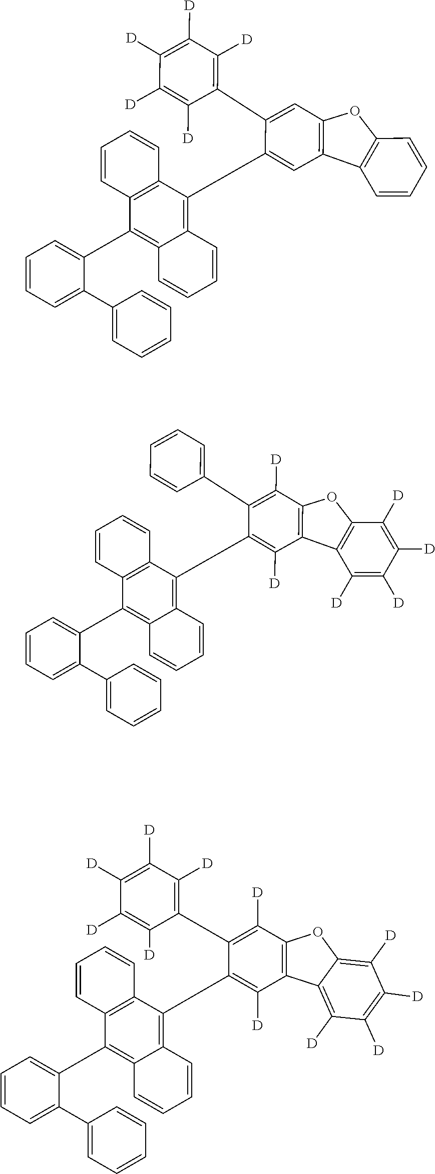 [SMILES: [2H]c1c([2H])c([2H])c(-c2cc3oc4ccccc4c3cc2-c2c3ccccc3c(-c3ccccc3-c3ccccc3)c3ccccc23)c([2H])c1[2H] ; [2H]c1c([2H])c([2H])c2c(oc3c([2H])c(-c4ccccc4)c(-c4c5ccccc5c(-c5ccccc5-c5ccccc5)c5ccccc45)c([2H])c32)c1[2H] ; [2H]c1c([2H])c([2H])c(-c2c(-c3c4ccccc4c(-c4ccccc4-c4ccccc4)c4ccccc34)c([2H])c3c(oc4c([2H])c([2H])c([2H])c([2H])c43)c2[2H])c([2H])c1[2H]]

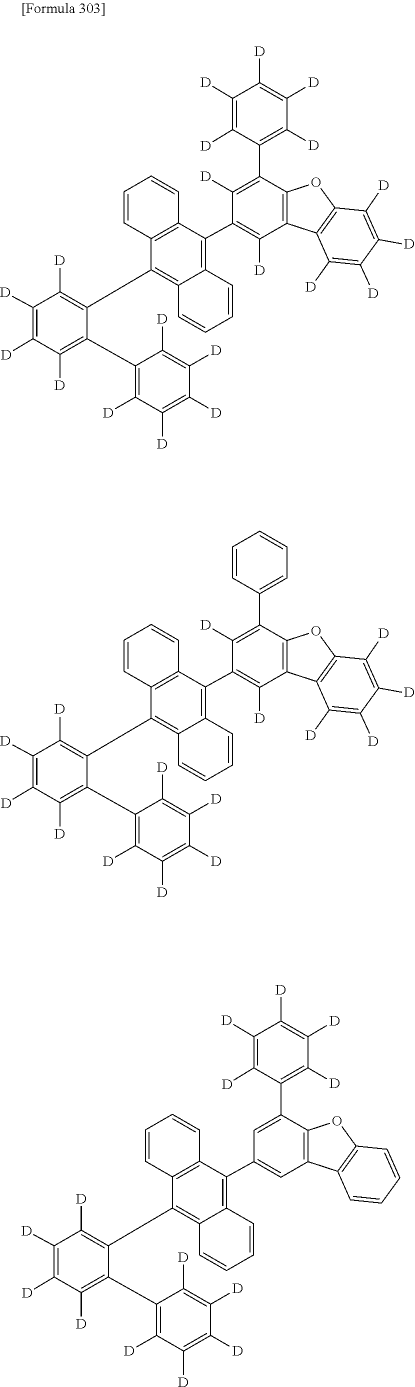 [SMILES: [2H]c1c([2H])c([2H])c(-c2c([2H])c([2H])c([2H])c([2H])c2-c2c3ccccc3c(-c3c([2H])c(-c4c([2H])c([2H])c([2H])c([2H])c4[2H])c4oc5c([2H])c([2H])c([2H])c([2H])c5c4c3[2H])c3ccccc23)c([2H])c1[2H] ; [2H]c1c([2H])c([2H])c(-c2c([2H])c([2H])c([2H])c([2H])c2-c2c3ccccc3c(-c3c([2H])c(-c4ccccc4)c4oc5c([2H])c([2H])c([2H])c([2H])c5c4c3[2H])c3ccccc23)c([2H])c1[2H] ; [2H]c1c([2H])c([2H])c(-c2c([2H])c([2H])c([2H])c([2H])c2-c2c3ccccc3c(-c3cc(-c4c([2H])c([2H])c([2H])c([2H])c4[2H])c4oc5ccccc5c4c3)c3ccccc23)c([2H])c1[2H]]

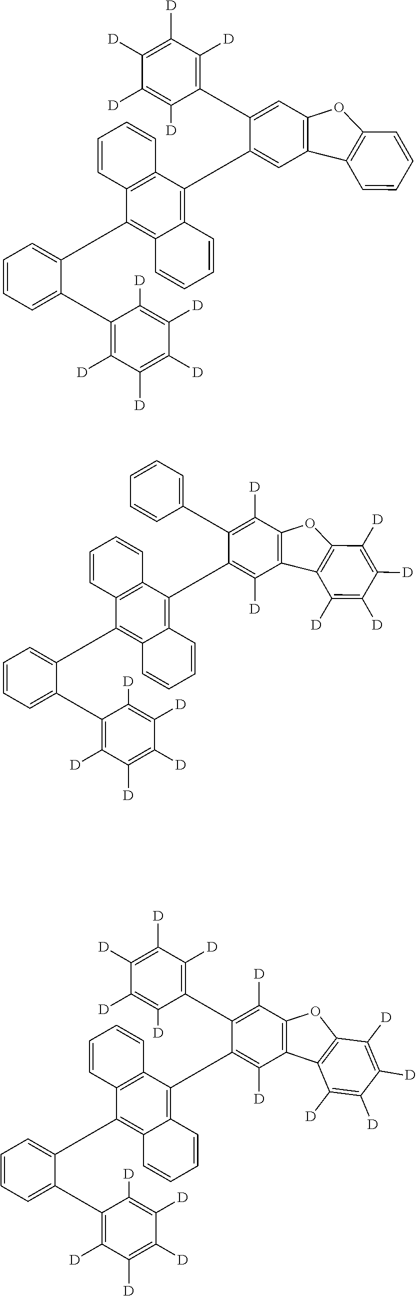 [SMILES: [2H]c1c([2H])c([2H])c(-c2ccccc2-c2c3ccccc3c(-c3cc4c(cc3-c3c([2H])c([2H])c([2H])c([2H])c3[2H])oc3ccccc34)c3ccccc23)c([2H])c1[2H] ; [2H]c1c([2H])c([2H])c(-c2ccccc2-c2c3ccccc3c(-c3c(-c4ccccc4)c([2H])c4oc5c([2H])c([2H])c([2H])c([2H])c5c4c3[2H])c3ccccc23)c([2H])c1[2H] ; [2H]c1c([2H])c([2H])c(-c2ccccc2-c2c3ccccc3c(-c3c(-c4c([2H])c([2H])c([2H])c([2H])c4[2H])c([2H])c4oc5c([2H])c([2H])c([2H])c([2H])c5c4c3[2H])c3ccccc23)c([2H])c1[2H]]

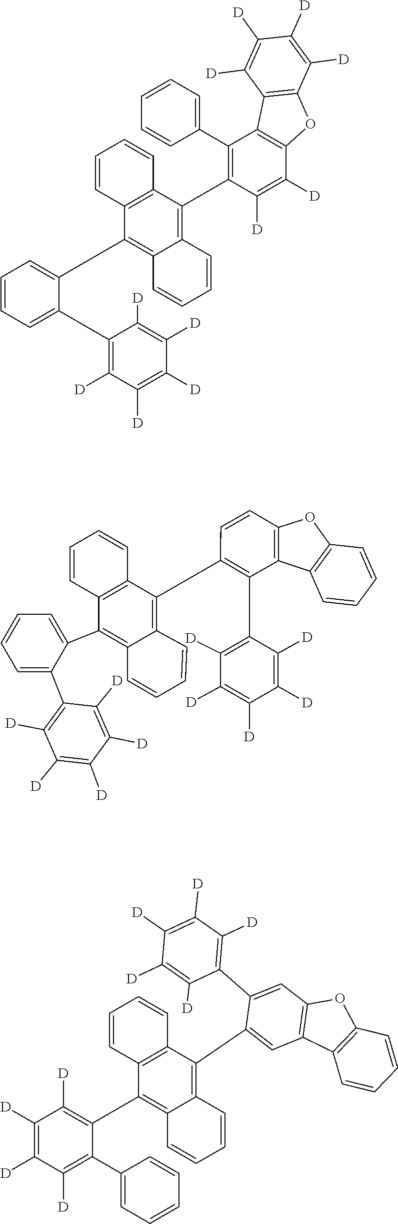 [SMILES: [2H]c1c([2H])c([2H])c(-c2ccccc2-c2c3ccccc3c(-c3c([2H])c([2H])c4oc5c([2H])c([2H])c([2H])c([2H])c5c4c3-c3ccccc3)c3ccccc23)c([2H])c1[2H] ; [2H]c1c([2H])c([2H])c(-c2ccccc2-c2c3ccccc3c(-c3ccc4oc5ccccc5c4c3-c3c([2H])c([2H])c([2H])c([2H])c3[2H])c3ccccc23)c([2H])c1[2H] ; [2H]c1c([2H])c([2H])c(-c2cc3oc4ccccc4c3cc2-c2c3ccccc3c(-c3c([2H])c([2H])c([2H])c([2H])c3-c3ccccc3)c3ccccc23)c([2H])c1[2H]]

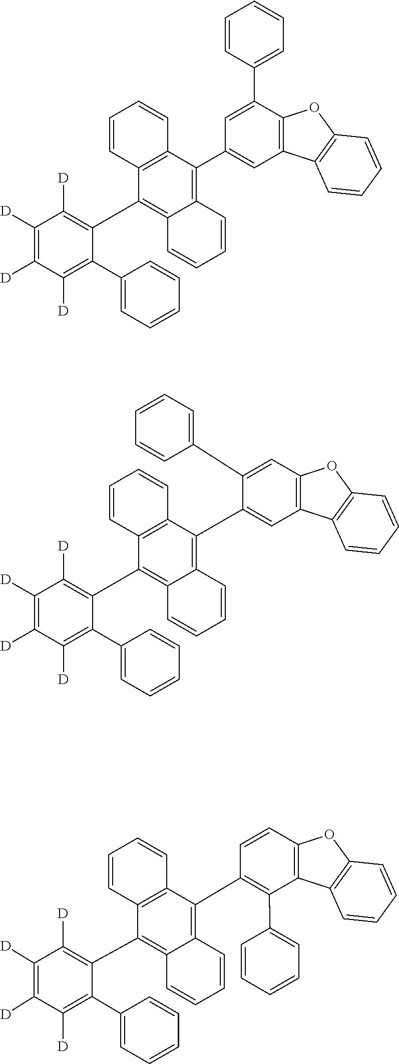 [SMILES: [2H]c1c([2H])c([2H])c(-c2c3ccccc3c(-c3cc(-c4ccccc4)c4oc5ccccc5c4c3)c3ccccc23)c(-c2ccccc2)c1[2H] ; [2H]c1c([2H])c([2H])c(-c2c3ccccc3c(-c3cc4c(cc3-c3ccccc3)oc3ccccc34)c3ccccc23)c(-c2ccccc2)c1[2H] ; [2H]c1c([2H])c([2H])c(-c2c3ccccc3c(-c3ccc4oc5ccccc5c4c3-c3ccccc3)c3ccccc23)c(-c2ccccc2)c1[2H]]

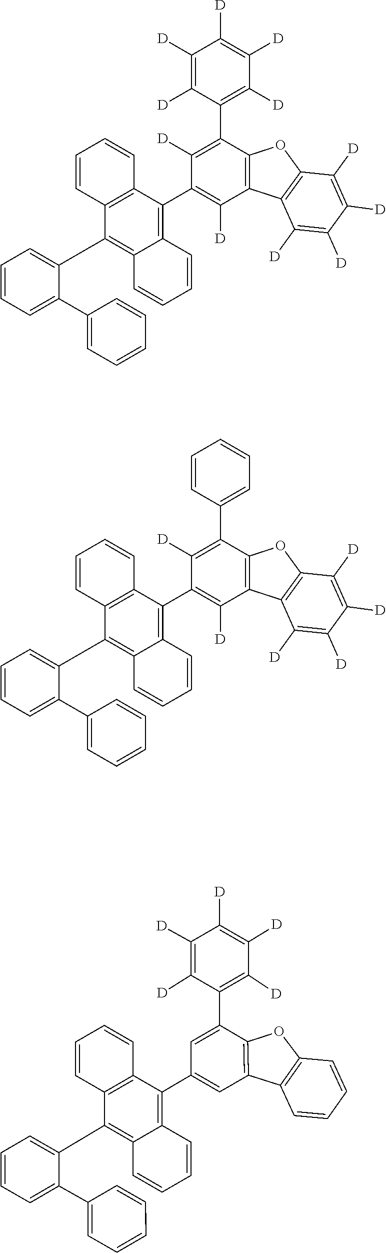 [SMILES: [2H]c1c([2H])c([2H])c(-c2c([2H])c(-c3c4ccccc4c(-c4ccccc4-c4ccccc4)c4ccccc34)c([2H])c3c2oc2c([2H])c([2H])c([2H])c([2H])c23)c([2H])c1[2H] ; [2H]c1c([2H])c([2H])c2c(oc3c(-c4ccccc4)c([2H])c(-c4c5ccccc5c(-c5ccccc5-c5ccccc5)c5ccccc45)c([2H])c32)c1[2H] ; [2H]c1c([2H])c([2H])c(-c2cc(-c3c4ccccc4c(-c4ccccc4-c4ccccc4)c4ccccc34)cc3c2oc2ccccc23)c([2H])c1[2H]]

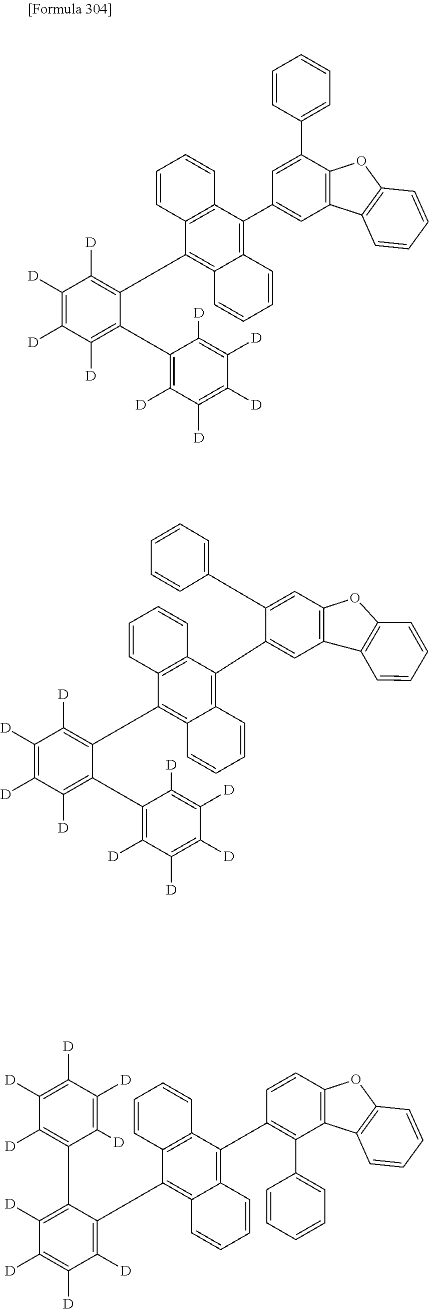 [SMILES: [2H]c1c([2H])c([2H])c(-c2c([2H])c([2H])c([2H])c([2H])c2-c2c3ccccc3c(-c3cc(-c4ccccc4)c4oc5ccccc5c4c3)c3ccccc23)c([2H])c1[2H] ; [2H]c1c([2H])c([2H])c(-c2c([2H])c([2H])c([2H])c([2H])c2-c2c3ccccc3c(-c3cc4c(cc3-c3ccccc3)oc3ccccc34)c3ccccc23)c([2H])c1[2H] ; [2H]c1c([2H])c([2H])c(-c2c([2H])c([2H])c([2H])c([2H])c2-c2c3ccccc3c(-c3ccc4oc5ccccc5c4c3-c3ccccc3)c3ccccc23)c([2H])c1[2H]]

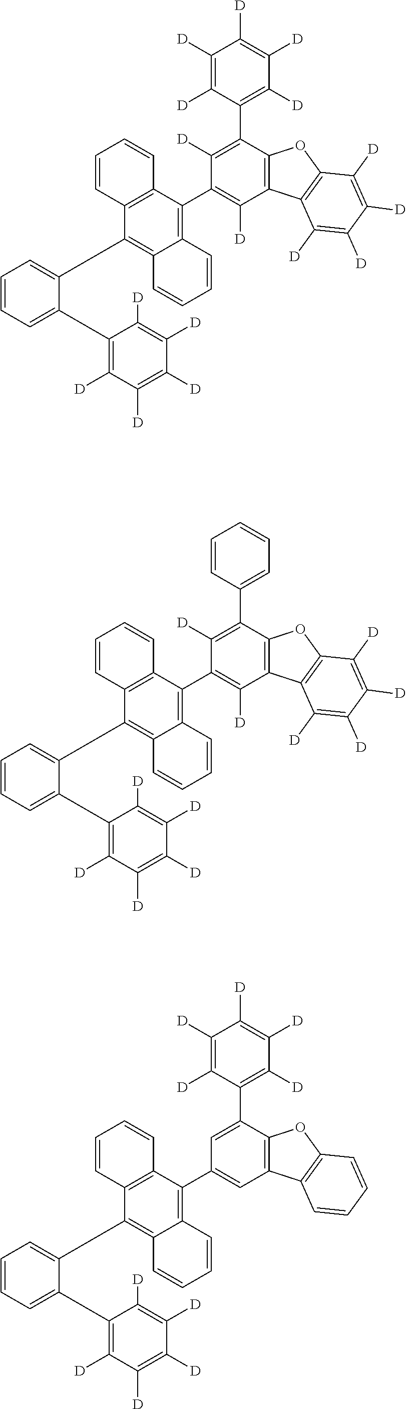 [SMILES: [2H]c1c([2H])c([2H])c(-c2ccccc2-c2c3ccccc3c(-c3c([2H])c(-c4c([2H])c([2H])c([2H])c([2H])c4[2H])c4oc5c([2H])c([2H])c([2H])c([2H])c5c4c3[2H])c3ccccc23)c([2H])c1[2H] ; [2H]c1c([2H])c([2H])c(-c2ccccc2-c2c3ccccc3c(-c3c([2H])c(-c4ccccc4)c4oc5c([2H])c([2H])c([2H])c([2H])c5c4c3[2H])c3ccccc23)c([2H])c1[2H] ; [2H]c1c([2H])c([2H])c(-c2ccccc2-c2c3ccccc3c(-c3cc(-c4c([2H])c([2H])c([2H])c([2H])c4[2H])c4oc5ccccc5c4c3)c3ccccc23)c([2H])c1[2H]]

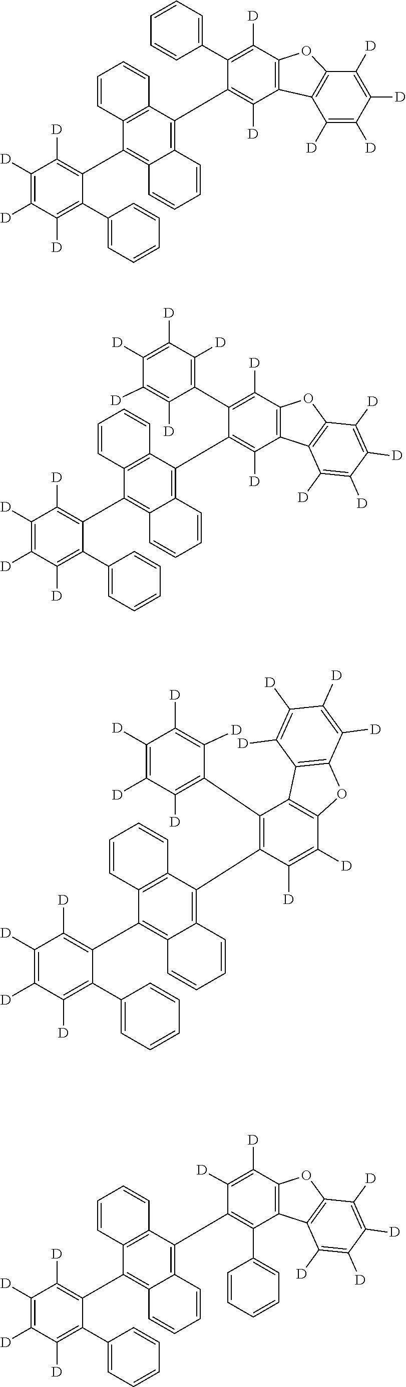

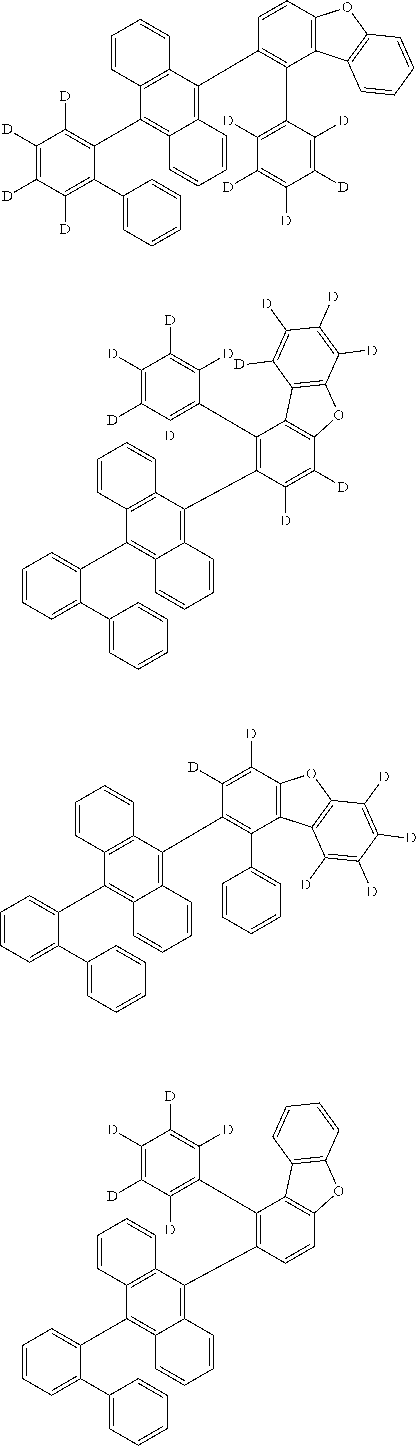

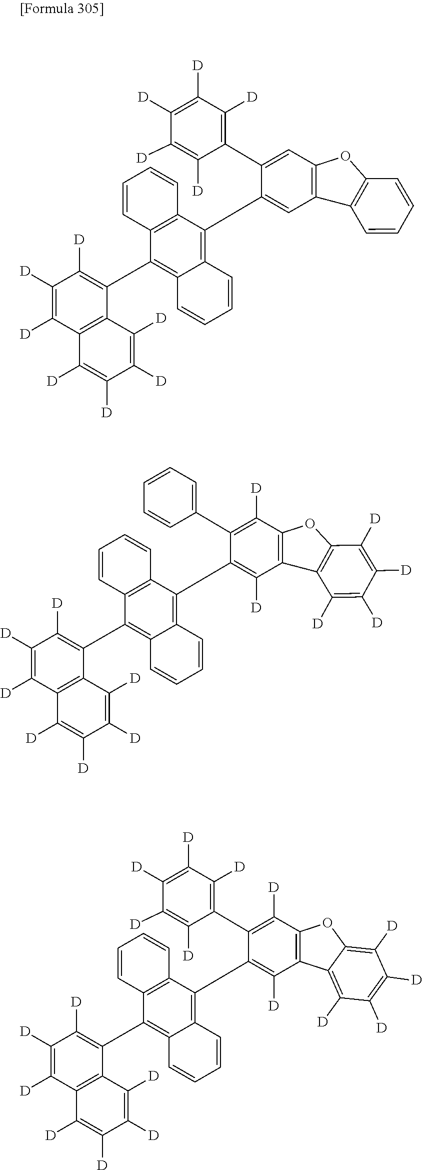 [SMILES: [2H]c1c([2H])c([2H])c(-c2cc3oc4ccccc4c3cc2-c2c3ccccc3c(-c3c([2H])c([2H])c([2H])c4c([2H])c([2H])c([2H])c([2H])c34)c3ccccc23)c([2H])c1[2H] ; [2H]c1c([2H])c([2H])c2c(oc3c([2H])c(-c4ccccc4)c(-c4c5ccccc5c(-c5c([2H])c([2H])c([2H])c6c([2H])c([2H])c([2H])c([2H])c56)c5ccccc45)c([2H])c32)c1[2H] ; [2H]c1c([2H])c([2H])c(-c2c(-c3c4ccccc4c(-c4c([2H])c([2H])c([2H])c5c([2H])c([2H])c([2H])c([2H])c45)c4ccccc34)c([2H])c3c(oc4c([2H])c([2H])c([2H])c([2H])c43)c2[2H])c([2H])c1[2H]]

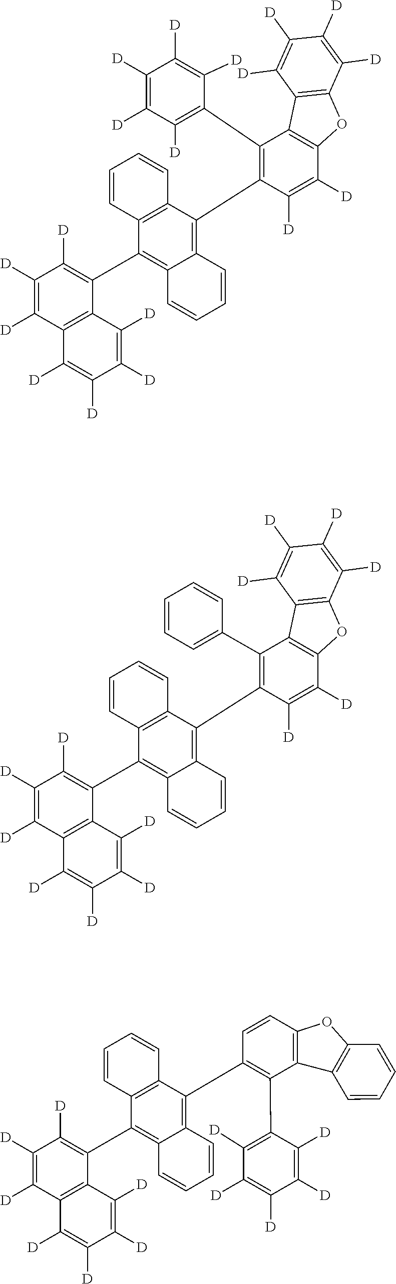 [SMILES: [2H]c1c([2H])c([2H])c(-c2c(-c3c4ccccc4c(-c4c([2H])c([2H])c([2H])c5c([2H])c([2H])c([2H])c([2H])c45)c4ccccc34)c([2H])c([2H])c3oc4c([2H])c([2H])c([2H])c([2H])c4c23)c([2H])c1[2H] ; [2H]c1c([2H])c([2H])c2c(oc3c([2H])c([2H])c(-c4c5ccccc5c(-c5c([2H])c([2H])c([2H])c6c([2H])c([2H])c([2H])c([2H])c56)c5ccccc45)c(-c4ccccc4)c32)c1[2H] ; [2H]c1c([2H])c([2H])c(-c2c(-c3c4ccccc4c(-c4c([2H])c([2H])c([2H])c5c([2H])c([2H])c([2H])c([2H])c45)c4ccccc34)ccc3oc4ccccc4c23)c([2H])c1[2H]]

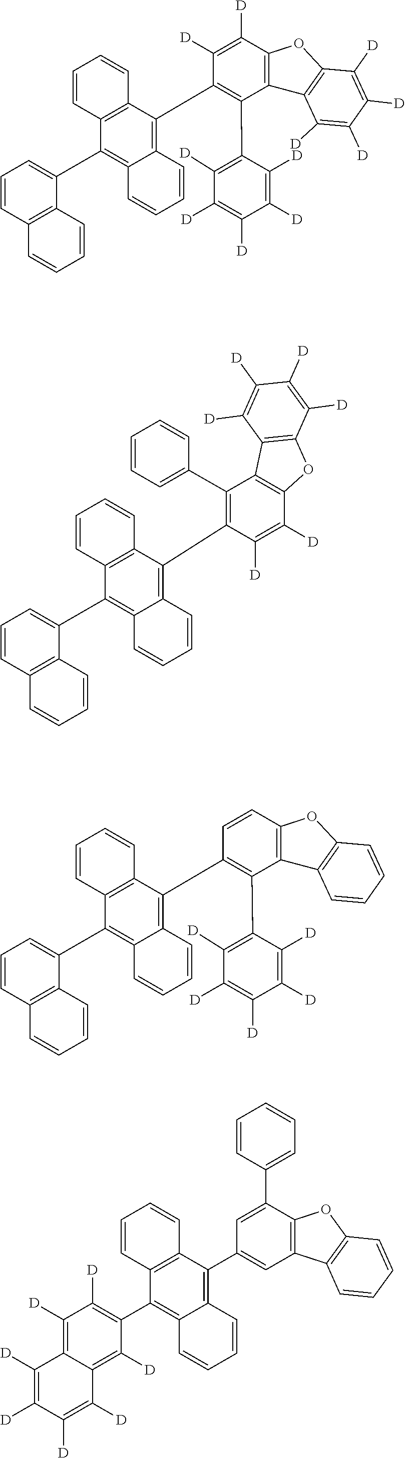 [SMILES: [2H]c1c([2H])c([2H])c(-c2c(-c3c4ccccc4c(-c4cccc5ccccc45)c4ccccc34)c([2H])c([2H])c3oc4c([2H])c([2H])c([2H])c([2H])c4c23)c([2H])c1[2H] ; [2H]c1c([2H])c([2H])c2c(oc3c([2H])c([2H])c(-c4c5ccccc5c(-c5cccc6ccccc56)c5ccccc45)c(-c4ccccc4)c32)c1[2H] ; [2H]c1c([2H])c([2H])c(-c2c(-c3c4ccccc4c(-c4cccc5ccccc45)c4ccccc34)ccc3oc4ccccc4c23)c([2H])c1[2H] ; [2H]c1c([2H])c([2H])c2c([2H])c(-c3c4ccccc4c(-c4cc(-c5ccccc5)c5oc6ccccc6c5c4)c4ccccc34)c([2H])c([2H])c2c1[2H]]

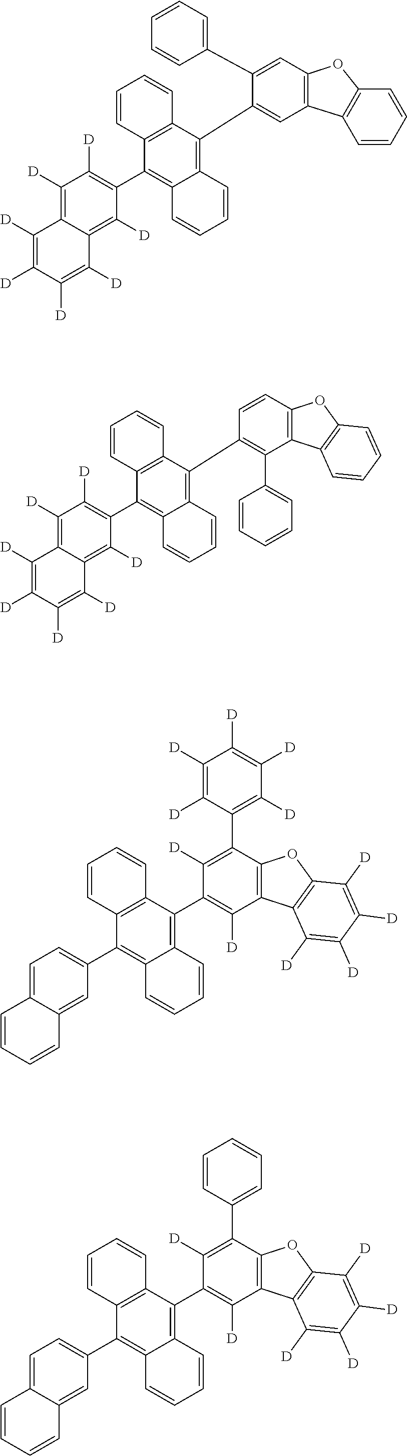 [SMILES: [2H]c1c([2H])c([2H])c2c([2H])c(-c3c4ccccc4c(-c4cc5c(cc4-c4ccccc4)oc4ccccc45)c4ccccc34)c([2H])c([2H])c2c1[2H] ; [2H]c1c([2H])c([2H])c2c([2H])c(-c3c4ccccc4c(-c4ccc5oc6ccccc6c5c4-c4ccccc4)c4ccccc34)c([2H])c([2H])c2c1[2H] ; [2H]c1c([2H])c([2H])c(-c2c([2H])c(-c3c4ccccc4c(-c4ccc5ccccc5c4)c4ccccc34)c([2H])c3c2oc2c([2H])c([2H])c([2H])c([2H])c23)c([2H])c1[2H] ; [2H]c1c([2H])c([2H])c2c(oc3c(-c4ccccc4)c([2H])c(-c4c5ccccc5c(-c5ccc6ccccc6c5)c5ccccc45)c([2H])c32)c1[2H]]

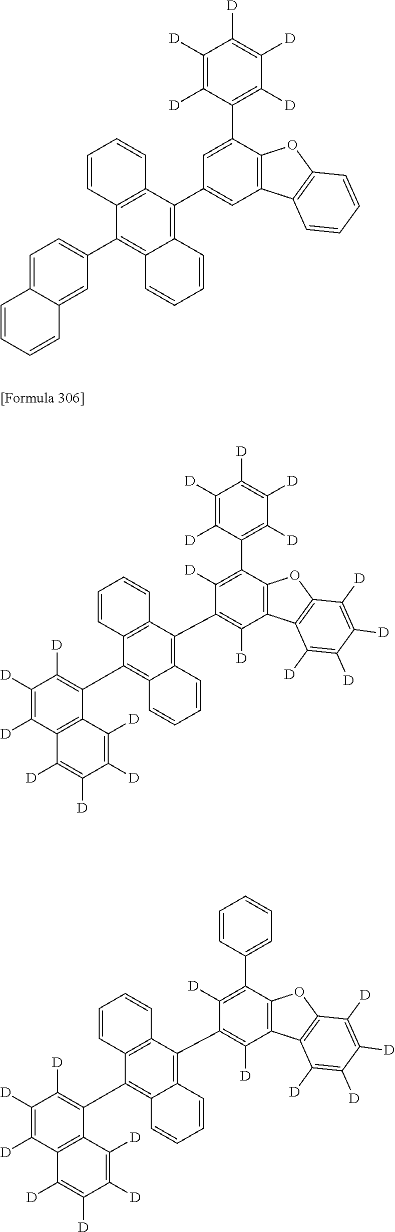 [SMILES: [2H]c1c([2H])c([2H])c(-c2cc(-c3c4ccccc4c(-c4ccc5ccccc5c4)c4ccccc34)cc3c2oc2ccccc23)c([2H])c1[2H] ; [2H]c1c([2H])c([2H])c(-c2c([2H])c(-c3c4ccccc4c(-c4c([2H])c([2H])c([2H])c5c([2H])c([2H])c([2H])c([2H])c45)c4ccccc34)c([2H])c3c2oc2c([2H])c([2H])c([2H])c([2H])c23)c([2H])c1[2H] ; [2H]c1c([2H])c([2H])c2c(oc3c(-c4ccccc4)c([2H])c(-c4c5ccccc5c(-c5c([2H])c([2H])c([2H])c6c([2H])c([2H])c([2H])c([2H])c56)c5ccccc45)c([2H])c32)c1[2H]]

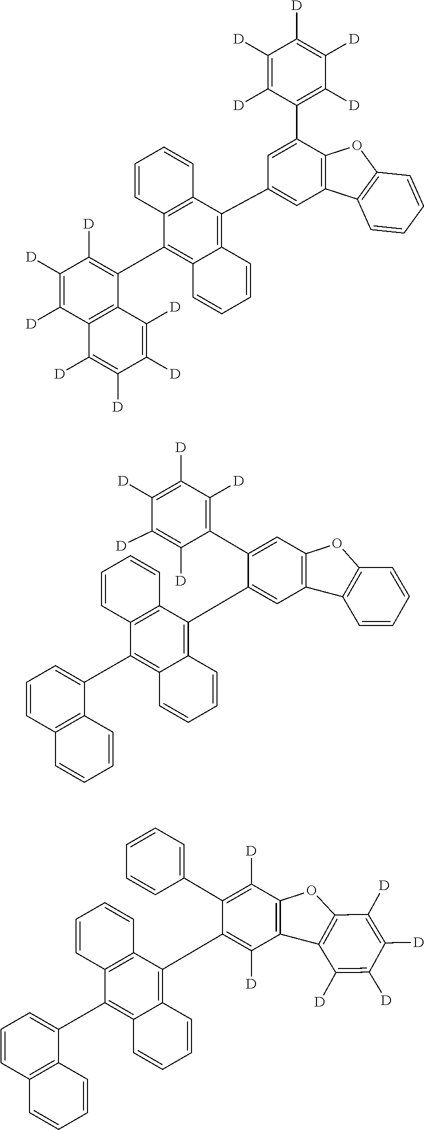 [SMILES: [2H]c1c([2H])c([2H])c(-c2cc(-c3c4ccccc4c(-c4c([2H])c([2H])c([2H])c5c([2H])c([2H])c([2H])c([2H])c45)c4ccccc34)cc3c2oc2ccccc23)c([2H])c1[2H] ; [2H]c1c([2H])c([2H])c(-c2cc3oc4ccccc4c3cc2-c2c3ccccc3c(-c3cccc4ccccc34)c3ccccc23)c([2H])c1[2H] ; [2H]c1c([2H])c([2H])c2c(oc3c([2H])c(-c4ccccc4)c(-c4c5ccccc5c(-c5cccc6ccccc56)c5ccccc45)c([2H])c32)c1[2H]]

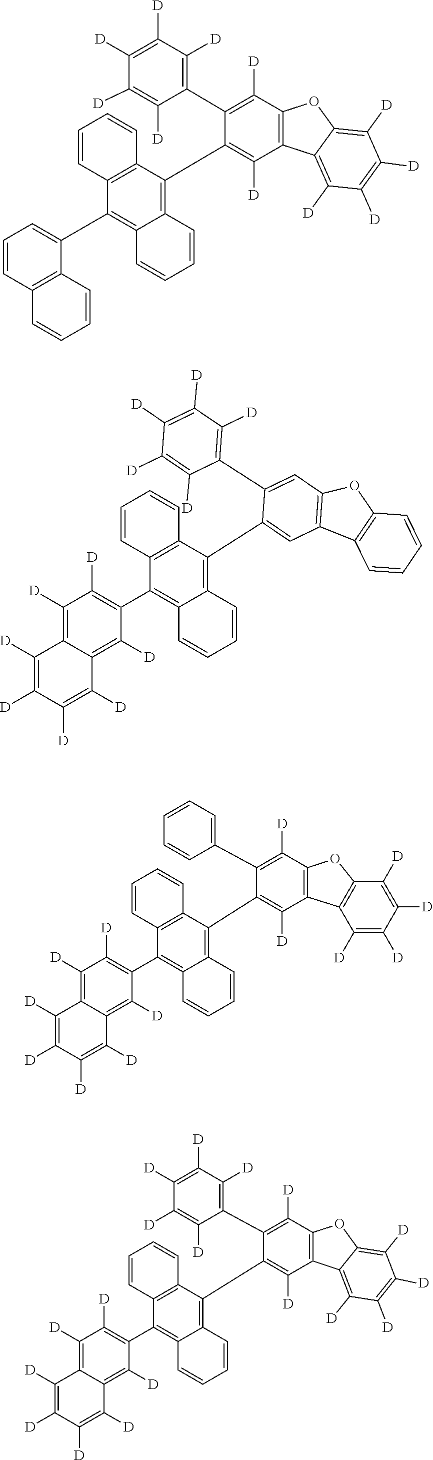

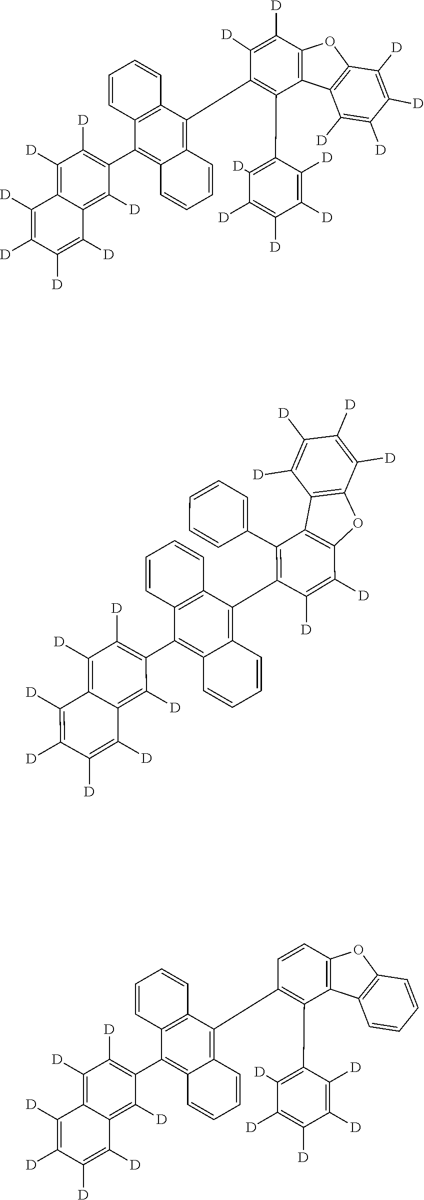 [SMILES: [2H]c1c([2H])c([2H])c(-c2c(-c3c4ccccc4c(-c4c([2H])c([2H])c5c([2H])c([2H])c([2H])c([2H])c5c4[2H])c4ccccc34)c([2H])c([2H])c3oc4c([2H])c([2H])c([2H])c([2H])c4c23)c([2H])c1[2H] ; [2H]c1c([2H])c([2H])c2c(oc3c([2H])c([2H])c(-c4c5ccccc5c(-c5c([2H])c([2H])c6c([2H])c([2H])c([2H])c([2H])c6c5[2H])c5ccccc45)c(-c4ccccc4)c32)c1[2H] ; [2H]c1c([2H])c([2H])c(-c2c(-c3c4ccccc4c(-c4c([2H])c([2H])c5c([2H])c([2H])c([2H])c([2H])c5c4[2H])c4ccccc34)ccc3oc4ccccc4c23)c([2H])c1[2H]]

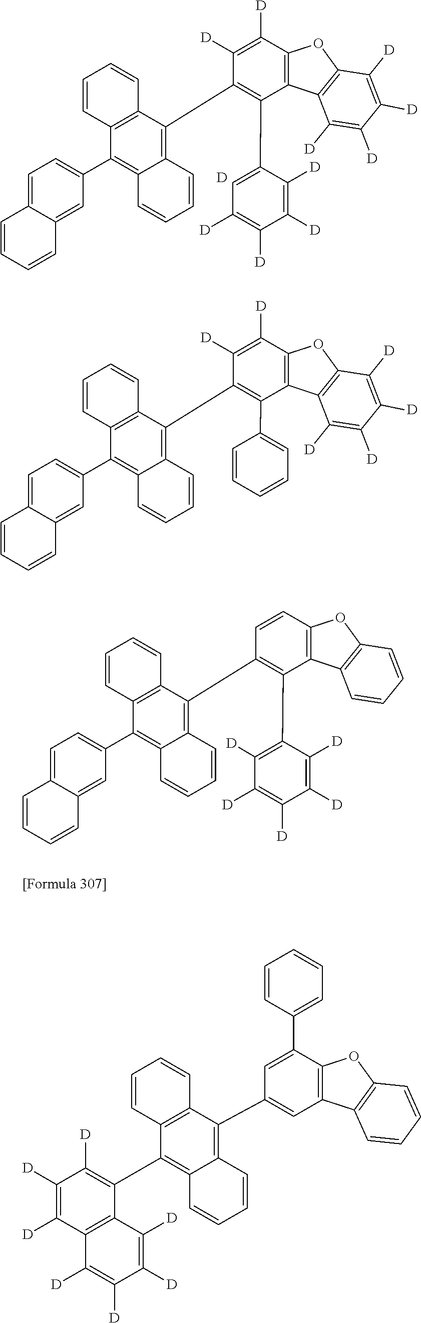

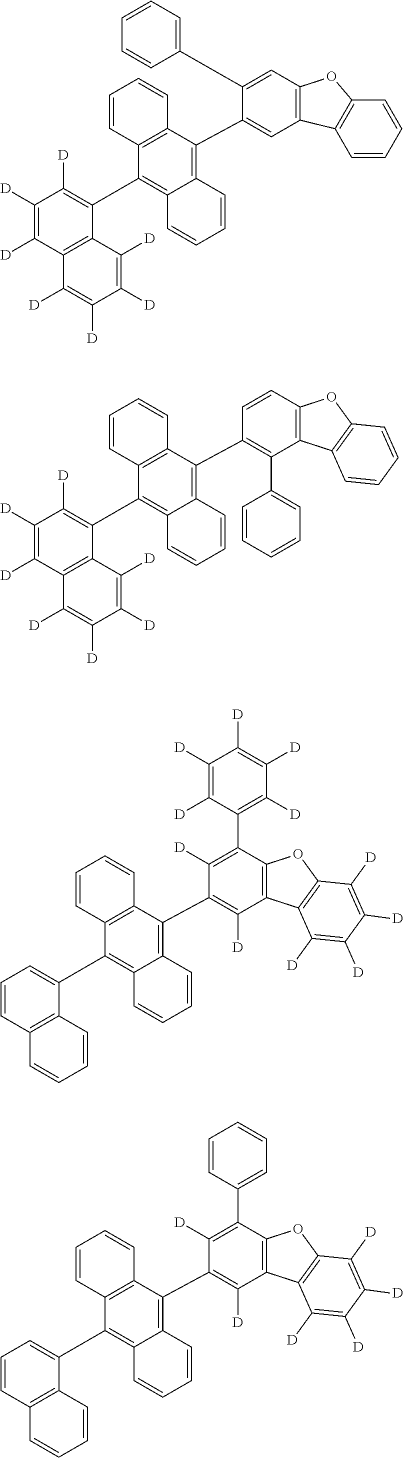 [SMILES: [2H]c1c([2H])c([2H])c2c(-c3c4ccccc4c(-c4cc5c(cc4-c4ccccc4)oc4ccccc45)c4ccccc34)c([2H])c([2H])c([2H])c2c1[2H] ; [2H]c1c([2H])c([2H])c2c(-c3c4ccccc4c(-c4ccc5oc6ccccc6c5c4-c4ccccc4)c4ccccc34)c([2H])c([2H])c([2H])c2c1[2H] ; [2H]c1c([2H])c([2H])c(-c2c([2H])c(-c3c4ccccc4c(-c4cccc5ccccc45)c4ccccc34)c([2H])c3c2oc2c([2H])c([2H])c([2H])c([2H])c23)c([2H])c1[2H] ; [2H]c1c([2H])c([2H])c2c(oc3c(-c4ccccc4)c([2H])c(-c4c5ccccc5c(-c5cccc6ccccc56)c5ccccc45)c([2H])c32)c1[2H]]

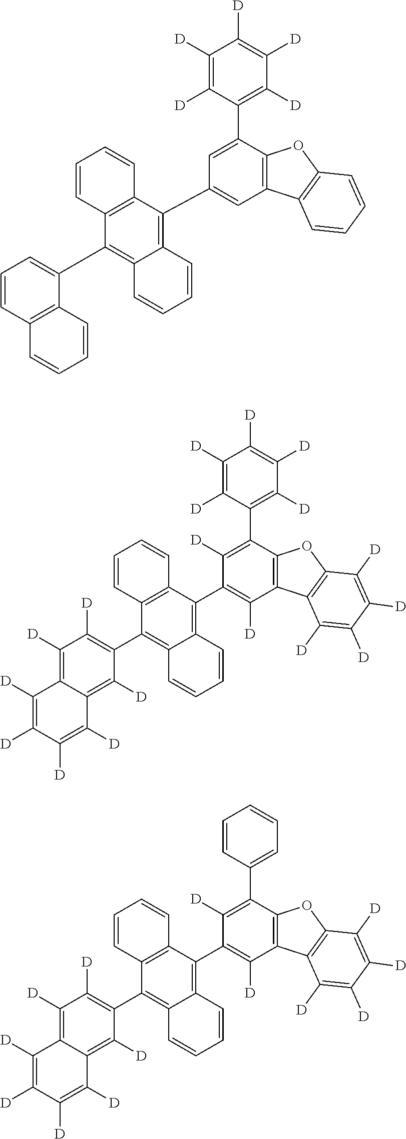 [SMILES: [2H]c1c([2H])c([2H])c(-c2cc(-c3c4ccccc4c(-c4cccc5ccccc45)c4ccccc34)cc3c2oc2ccccc23)c([2H])c1[2H] ; [2H]c1c([2H])c([2H])c(-c2c([2H])c(-c3c4ccccc4c(-c4c([2H])c([2H])c5c([2H])c([2H])c([2H])c([2H])c5c4[2H])c4ccccc34)c([2H])c3c2oc2c([2H])c([2H])c([2H])c([2H])c23)c([2H])c1[2H] ; [2H]c1c([2H])c([2H])c2c(oc3c(-c4ccccc4)c([2H])c(-c4c5ccccc5c(-c5c([2H])c([2H])c6c([2H])c([2H])c([2H])c([2H])c6c5[2H])c5ccccc45)c([2H])c32)c1[2H]]

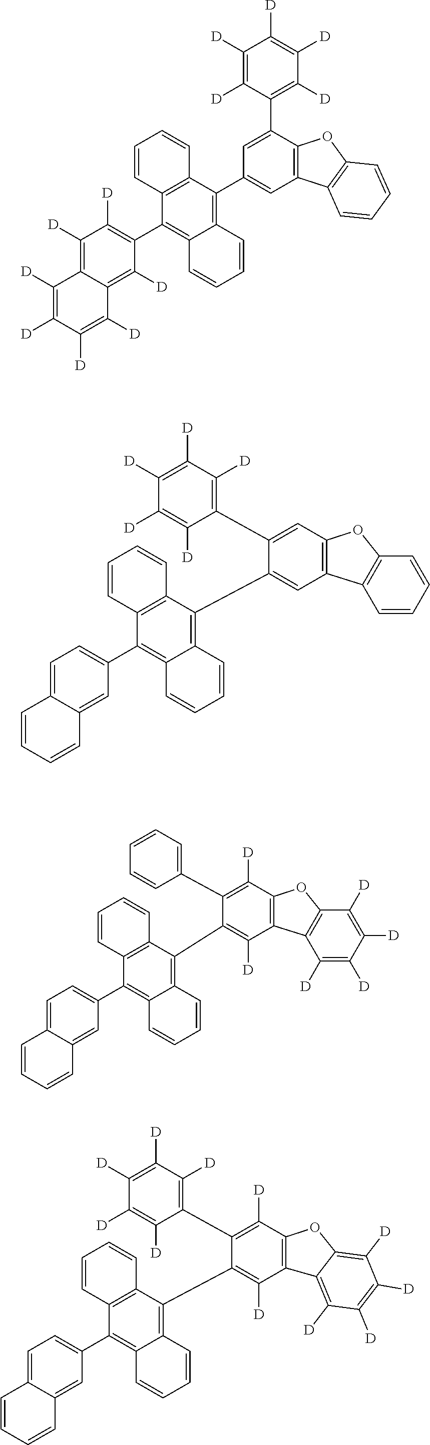 [SMILES: [2H]c1c([2H])c([2H])c(-c2cc(-c3c4ccccc4c(-c4c([2H])c([2H])c5c([2H])c([2H])c([2H])c([2H])c5c4[2H])c4ccccc34)cc3c2oc2ccccc23)c([2H])c1[2H] ; [2H]c1c([2H])c([2H])c(-c2cc3oc4ccccc4c3cc2-c2c3ccccc3c(-c3ccc4ccccc4c3)c3ccccc23)c([2H])c1[2H] ; [2H]c1c([2H])c([2H])c2c(oc3c([2H])c(-c4ccccc4)c(-c4c5ccccc5c(-c5ccc6ccccc6c5)c5ccccc45)c([2H])c32)c1[2H] ; [2H]c1c([2H])c([2H])c(-c2c(-c3c4ccccc4c(-c4ccc5ccccc5c4)c4ccccc34)c([2H])c3c(oc4c([2H])c([2H])c([2H])c([2H])c43)c2[2H])c([2H])c1[2H]]

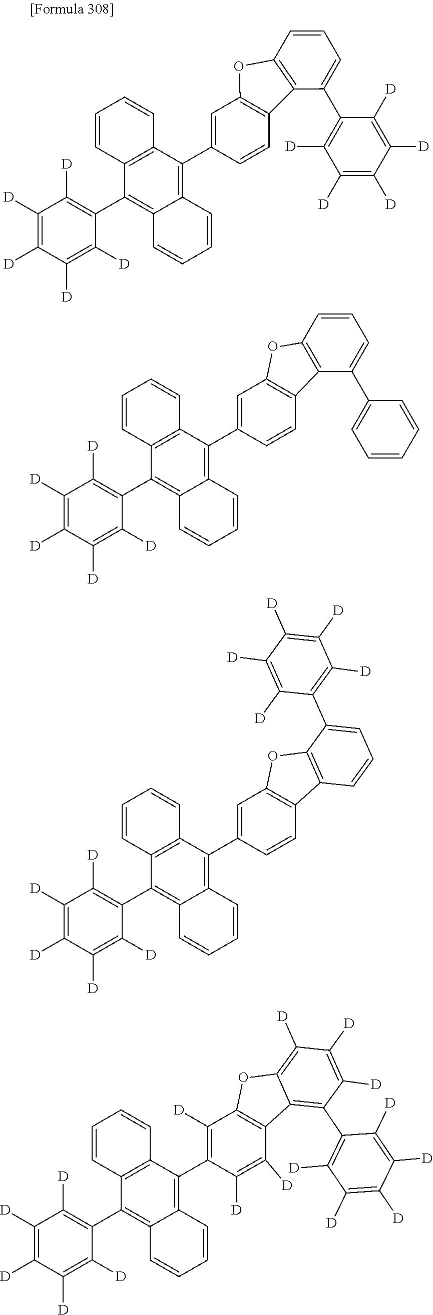 [SMILES: [2H]c1c([2H])c([2H])c(-c2c3ccccc3c(-c3ccc4c(c3)oc3cccc(-c5c([2H])c([2H])c([2H])c([2H])c5[2H])c34)c3ccccc23)c([2H])c1[2H] ; [2H]c1c([2H])c([2H])c(-c2c3ccccc3c(-c3ccc4c(c3)oc3cccc(-c5ccccc5)c34)c3ccccc23)c([2H])c1[2H] ; [2H]c1c([2H])c([2H])c(-c2c3ccccc3c(-c3ccc4c(c3)oc3c(-c5c([2H])c([2H])c([2H])c([2H])c5[2H])cccc34)c3ccccc23)c([2H])c1[2H] ; [2H]c1c([2H])c([2H])c(-c2c3ccccc3c(-c3c([2H])c([2H])c4c(oc5c([2H])c([2H])c([2H])c(-c6c([2H])c([2H])c([2H])c([2H])c6[2H])c54)c3[2H])c3ccccc23)c([2H])c1[2H]]

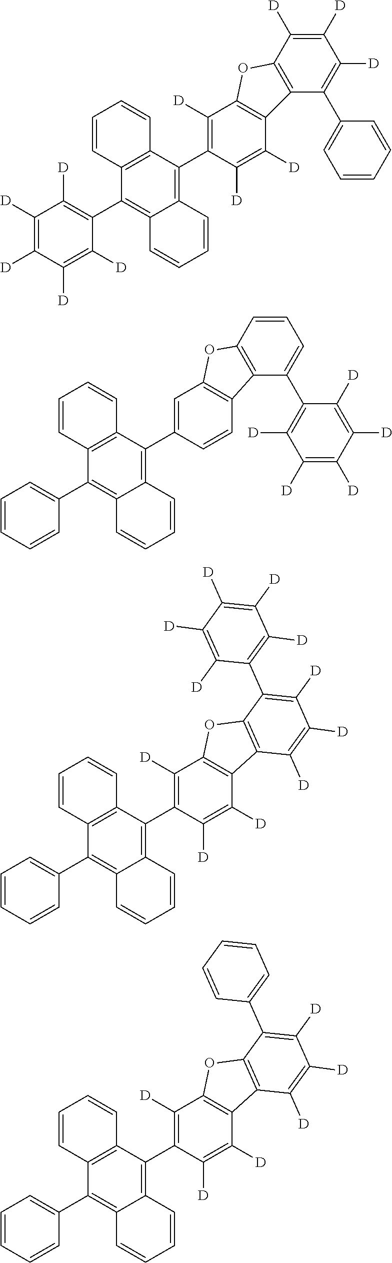 [SMILES: [2H]c1c([2H])c([2H])c(-c2c3ccccc3c(-c3c([2H])c([2H])c4c(oc5c([2H])c([2H])c([2H])c(-c6ccccc6)c54)c3[2H])c3ccccc23)c([2H])c1[2H] ; [2H]c1c([2H])c([2H])c(-c2cccc3oc4cc(-c5c6ccccc6c(-c6ccccc6)c6ccccc56)ccc4c23)c([2H])c1[2H] ; [2H]c1c([2H])c([2H])c(-c2c([2H])c([2H])c([2H])c3c2oc2c([2H])c(-c4c5ccccc5c(-c5ccccc5)c5ccccc45)c([2H])c([2H])c23)c([2H])c1[2H] ; [2H]c1c([2H])c([2H])c2c(oc3c([2H])c(-c4c5ccccc5c(-c5ccccc5)c5ccccc45)c([2H])c([2H])c32)c1-c1ccccc1]

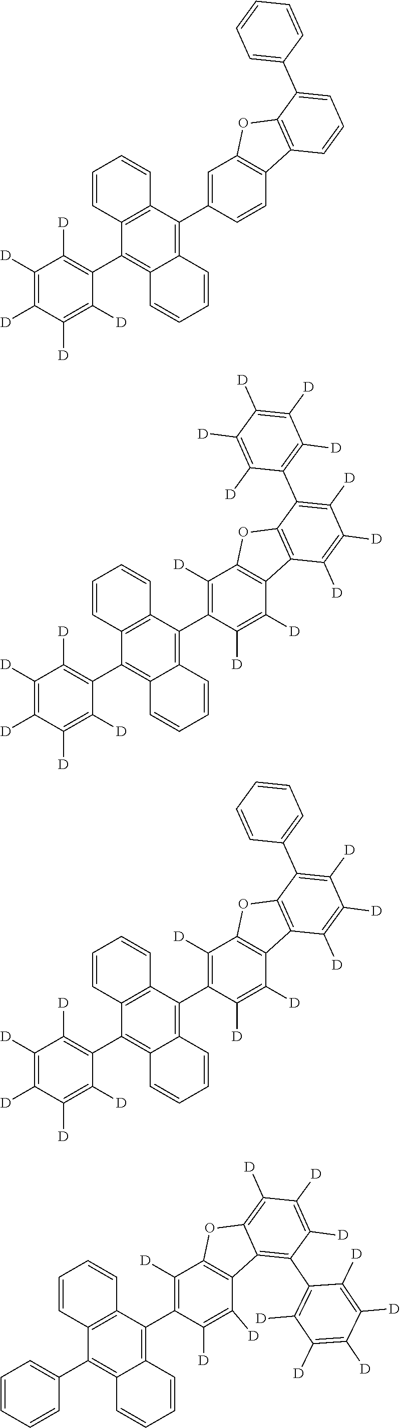

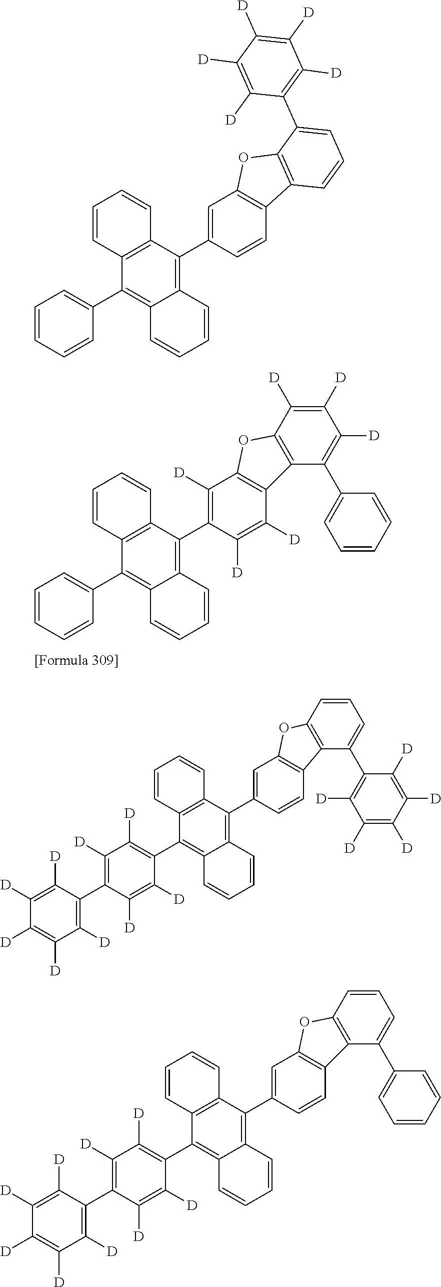 [SMILES: [2H]c1c([2H])c([2H])c(-c2cccc3c2oc2cc(-c4c5ccccc5c(-c5ccccc5)c5ccccc45)ccc23)c([2H])c1[2H] ; [2H]c1c([2H])c(-c2ccccc2)c2c(oc3c([2H])c(-c4c5ccccc5c(-c5ccccc5)c5ccccc45)c([2H])c([2H])c32)c1[2H] ; [2H]c1c([2H])c([2H])c(-c2c([2H])c([2H])c(-c3c4ccccc4c(-c4ccc5c(c4)oc4cccc(-c6c([2H])c([2H])c([2H])c([2H])c6[2H])c45)c4ccccc34)c([2H])c2[2H])c([2H])c1[2H] ; [2H]c1c([2H])c([2H])c(-c2c([2H])c([2H])c(-c3c4ccccc4c(-c4ccc5c(c4)oc4cccc(-c6ccccc6)c45)c4ccccc34)c([2H])c2[2H])c([2H])c1[2H]]

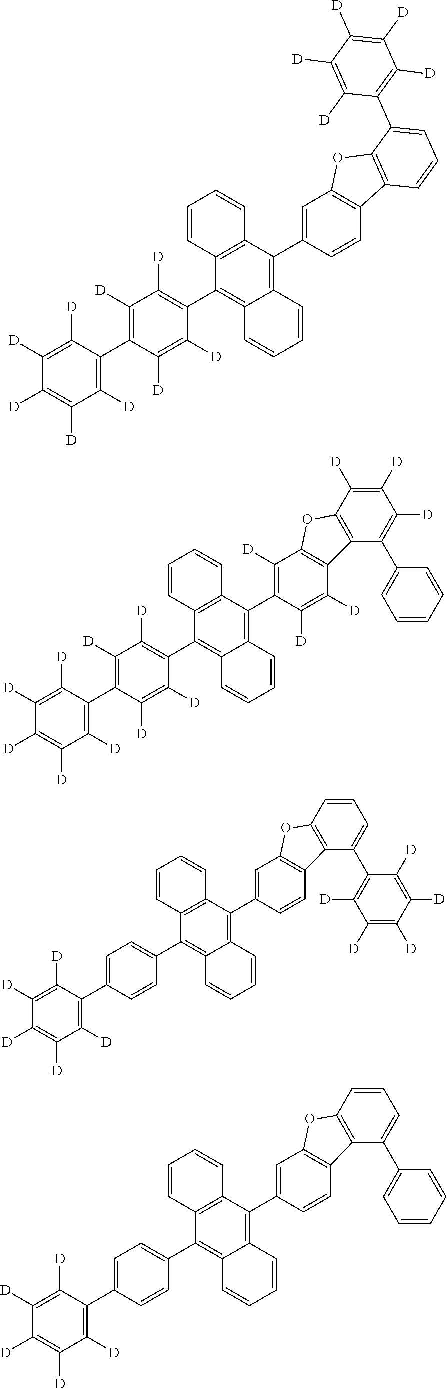

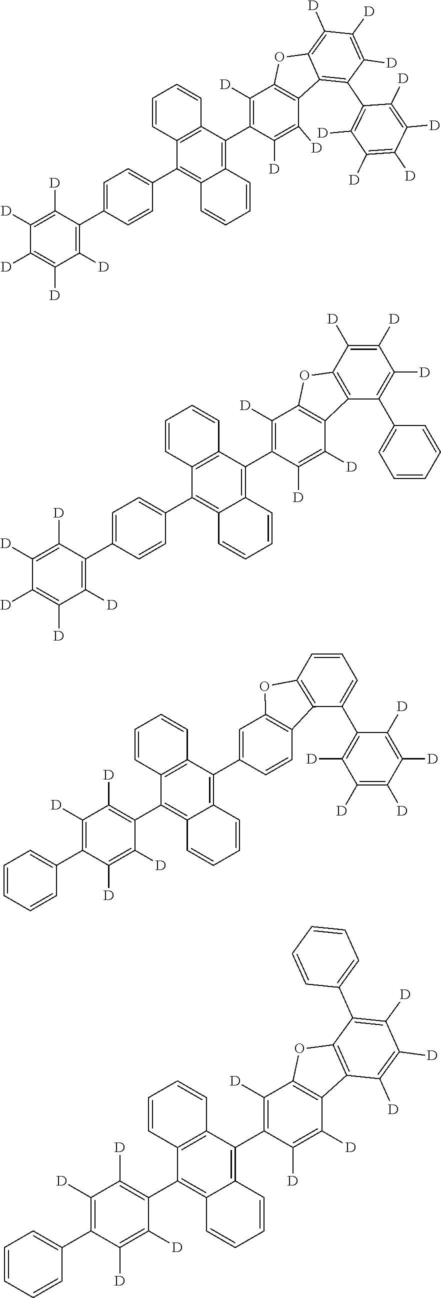

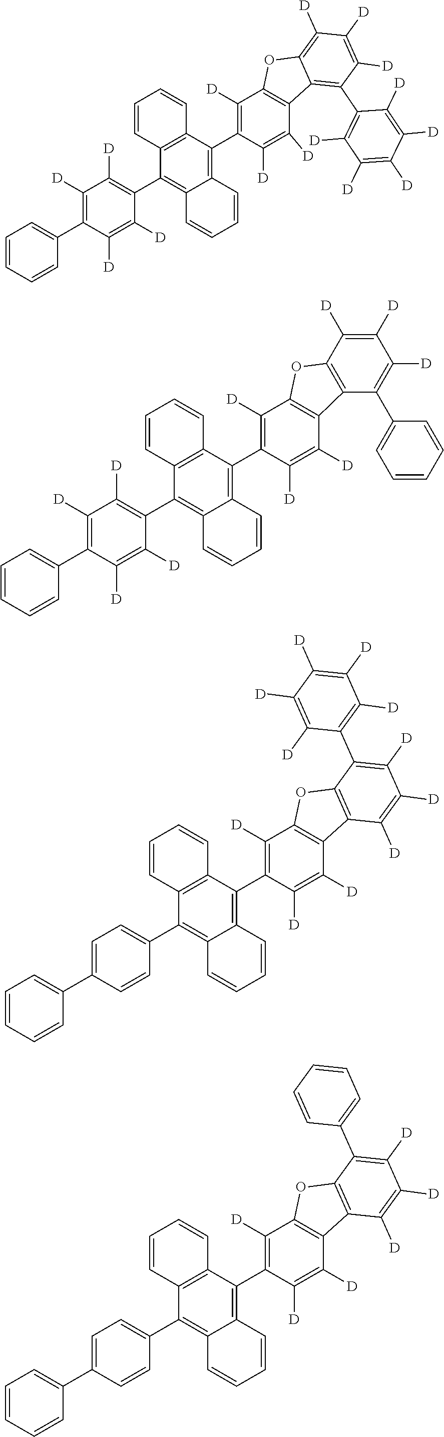

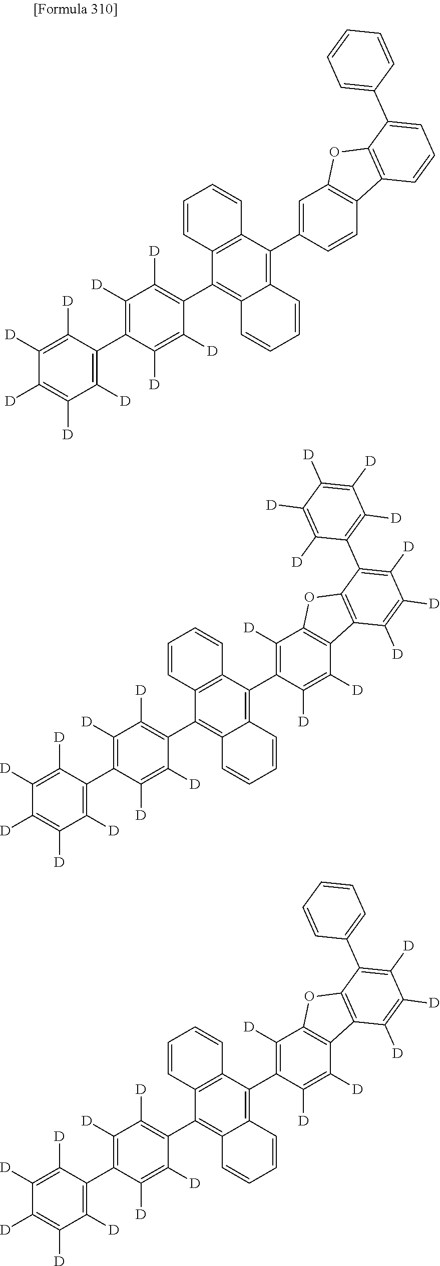 [SMILES: [2H]c1c([2H])c([2H])c(-c2c([2H])c([2H])c(-c3c4ccccc4c(-c4ccc5c(c4)oc4c(-c6ccccc6)cccc45)c4ccccc34)c([2H])c2[2H])c([2H])c1[2H] ; [2H]c1c([2H])c([2H])c(-c2c([2H])c([2H])c(-c3c4ccccc4c(-c4c([2H])c([2H])c5c(oc6c(-c7c([2H])c([2H])c([2H])c([2H])c7[2H])c([2H])c([2H])c([2H])c65)c4[2H])c4ccccc34)c([2H])c2[2H])c([2H])c1[2H] ; [2H]c1c([2H])c([2H])c(-c2c([2H])c([2H])c(-c3c4ccccc4c(-c4c([2H])c([2H])c5c(oc6c(-c7ccccc7)c([2H])c([2H])c([2H])c65)c4[2H])c4ccccc34)c([2H])c2[2H])c([2H])c1[2H]]

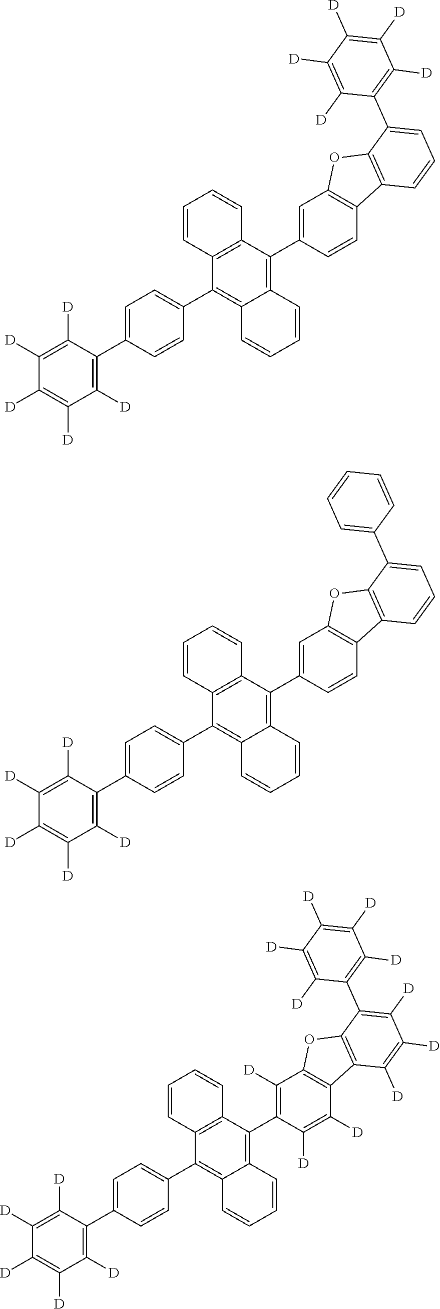 [SMILES: [2H]c1c([2H])c([2H])c(-c2ccc(-c3c4ccccc4c(-c4ccc5c(c4)oc4c(-c6c([2H])c([2H])c([2H])c([2H])c6[2H])cccc45)c4ccccc34)cc2)c([2H])c1[2H] ; [2H]c1c([2H])c([2H])c(-c2ccc(-c3c4ccccc4c(-c4ccc5c(c4)oc4c(-c6ccccc6)cccc45)c4ccccc34)cc2)c([2H])c1[2H] ; [2H]c1c([2H])c([2H])c(-c2ccc(-c3c4ccccc4c(-c4c([2H])c([2H])c5c(oc6c(-c7c([2H])c([2H])c([2H])c([2H])c7[2H])c([2H])c([2H])c([2H])c65)c4[2H])c4ccccc34)cc2)c([2H])c1[2H]]

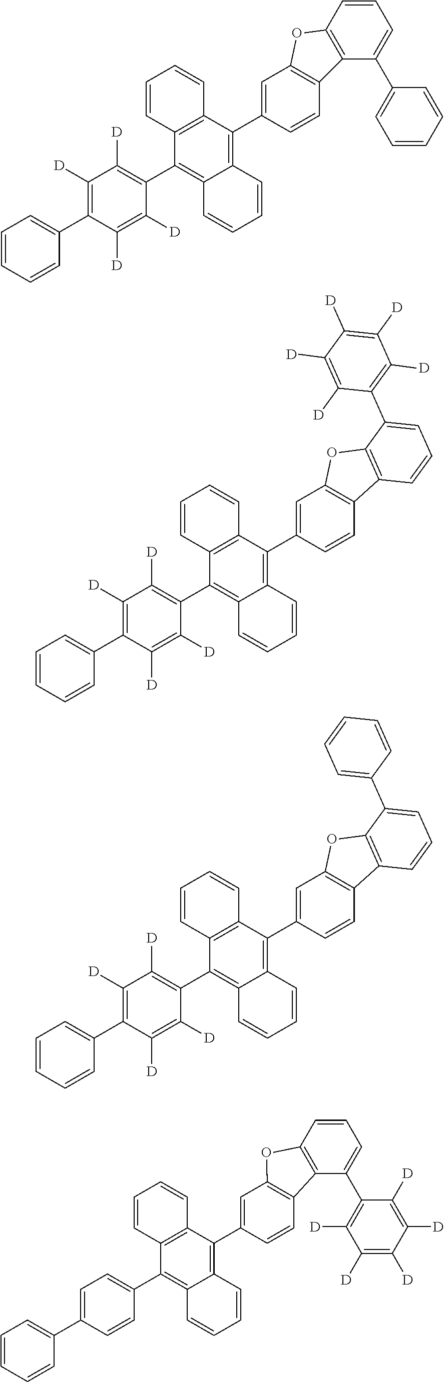 [SMILES: [2H]c1c([2H])c(-c2c3ccccc3c(-c3ccc4c(c3)oc3cccc(-c5ccccc5)c34)c3ccccc23)c([2H])c([2H])c1-c1ccccc1 ; [2H]c1c([2H])c([2H])c(-c2cccc3c2oc2cc(-c4c5ccccc5c(-c5c([2H])c([2H])c(-c6ccccc6)c([2H])c5[2H])c5ccccc45)ccc23)c([2H])c1[2H] ; [2H]c1c([2H])c(-c2c3ccccc3c(-c3ccc4c(c3)oc3c(-c5ccccc5)cccc34)c3ccccc23)c([2H])c([2H])c1-c1ccccc1 ; [2H]c1c([2H])c([2H])c(-c2cccc3oc4cc(-c5c6ccccc6c(-c6ccc(-c7ccccc7)cc6)c6ccccc56)ccc4c23)c([2H])c1[2H]]

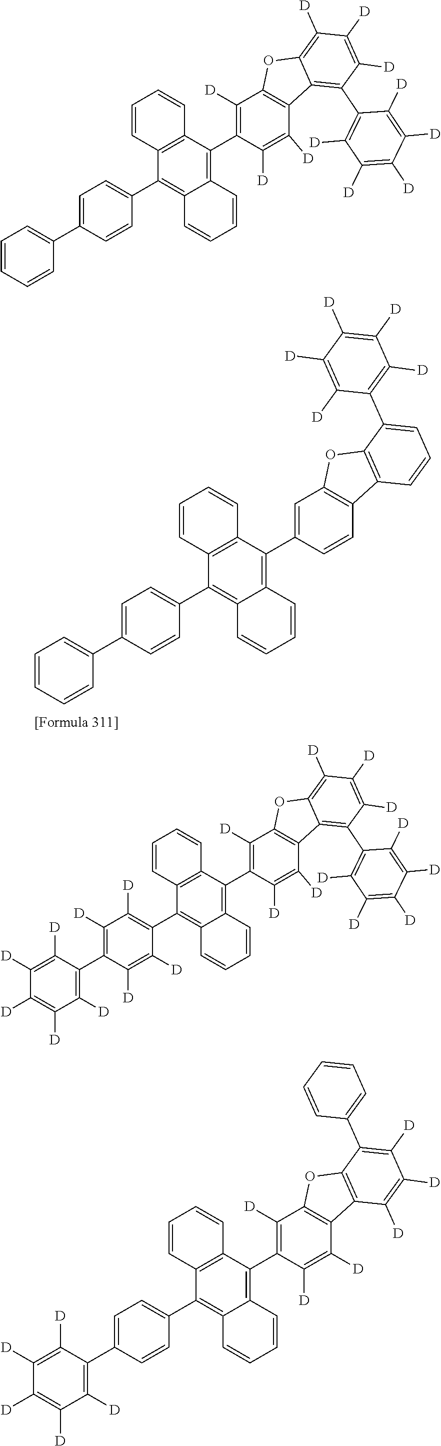

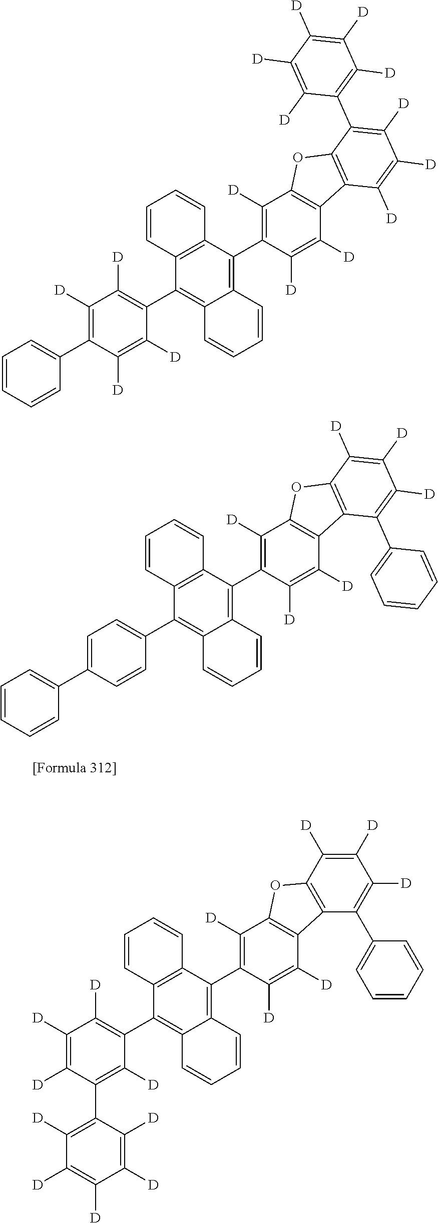 [SMILES: [2H]c1c([2H])c([2H])c(-c2c([2H])c([2H])c([2H])c3c2oc2c([2H])c(-c4c5ccccc5c(-c5c([2H])c([2H])c(-c6ccccc6)c([2H])c5[2H])c5ccccc45)c([2H])c([2H])c23)c([2H])c1[2H] ; [2H]c1c([2H])c(-c2ccccc2)c2c(oc3c([2H])c(-c4c5ccccc5c(-c5ccc(-c6ccccc6)cc5)c5ccccc45)c([2H])c([2H])c32)c1[2H] ; [2H]c1c([2H])c([2H])c(-c2c([2H])c([2H])c([2H])c(-c3c4ccccc4c(-c4c([2H])c([2H])c5c(oc6c([2H])c([2H])c([2H])c(-c7ccccc7)c65)c4[2H])c4ccccc34)c2[2H])c([2H])c1[2H]]

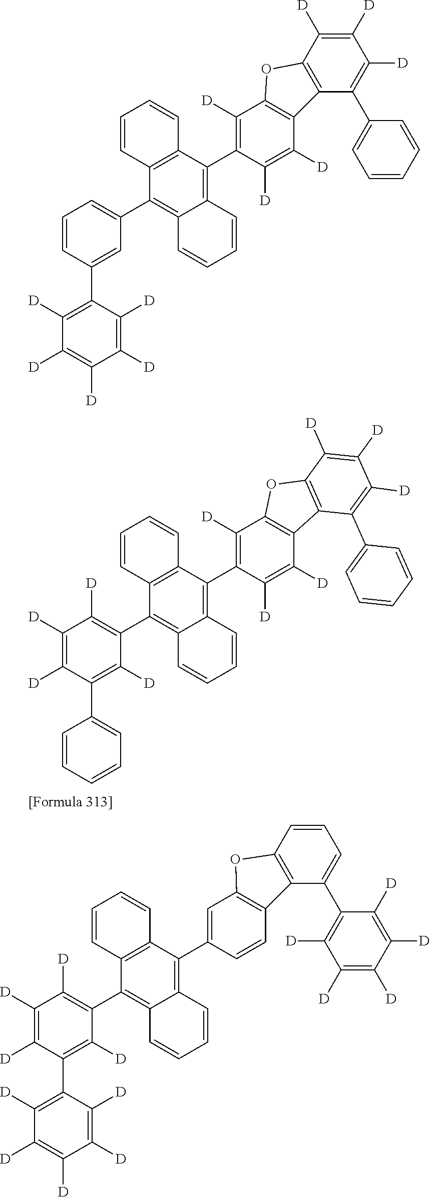 [SMILES: [2H]c1c([2H])c([2H])c(-c2cccc(-c3c4ccccc4c(-c4c([2H])c([2H])c5c(oc6c([2H])c([2H])c([2H])c(-c7ccccc7)c65)c4[2H])c4ccccc34)c2)c([2H])c1[2H] ; [2H]c1c([2H])c(-c2ccccc2)c([2H])c(-c2c3ccccc3c(-c3c([2H])c([2H])c4c(oc5c([2H])c([2H])c([2H])c(-c6ccccc6)c54)c3[2H])c3ccccc23)c1[2H] ; [2H]c1c([2H])c([2H])c(-c2c([2H])c([2H])c([2H])c(-c3c4ccccc4c(-c4ccc5c(c4)oc4cccc(-c6c([2H])c([2H])c([2H])c([2H])c6[2H])c45)c4ccccc34)c2[2H])c([2H])c1[2H]]

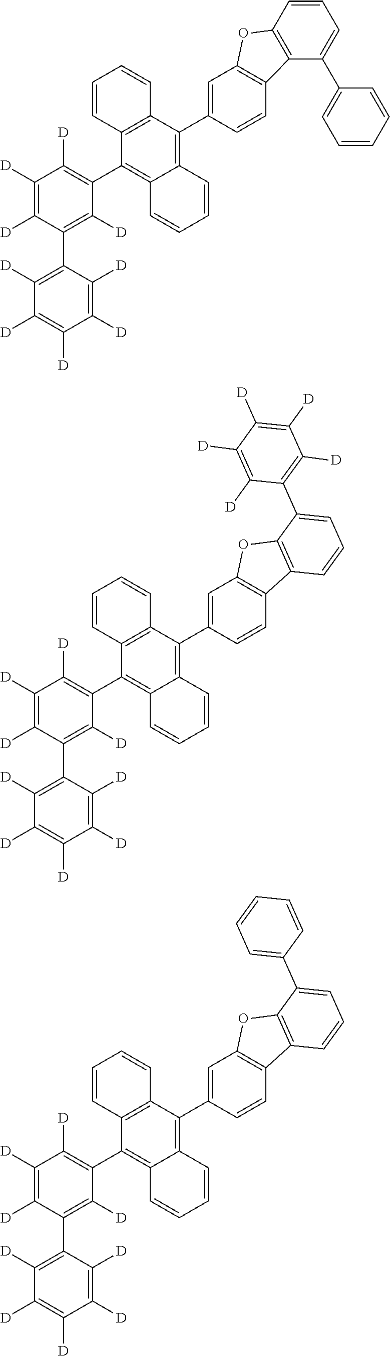 [SMILES: [2H]c1c([2H])c([2H])c(-c2c([2H])c([2H])c([2H])c(-c3c4ccccc4c(-c4ccc5c(c4)oc4cccc(-c6ccccc6)c45)c4ccccc34)c2[2H])c([2H])c1[2H] ; [2H]c1c([2H])c([2H])c(-c2c([2H])c([2H])c([2H])c(-c3c4ccccc4c(-c4ccc5c(c4)oc4c(-c6c([2H])c([2H])c([2H])c([2H])c6[2H])cccc45)c4ccccc34)c2[2H])c([2H])c1[2H] ; [2H]c1c([2H])c([2H])c(-c2c([2H])c([2H])c([2H])c(-c3c4ccccc4c(-c4ccc5c(c4)oc4c(-c6ccccc6)cccc45)c4ccccc34)c2[2H])c([2H])c1[2H]]

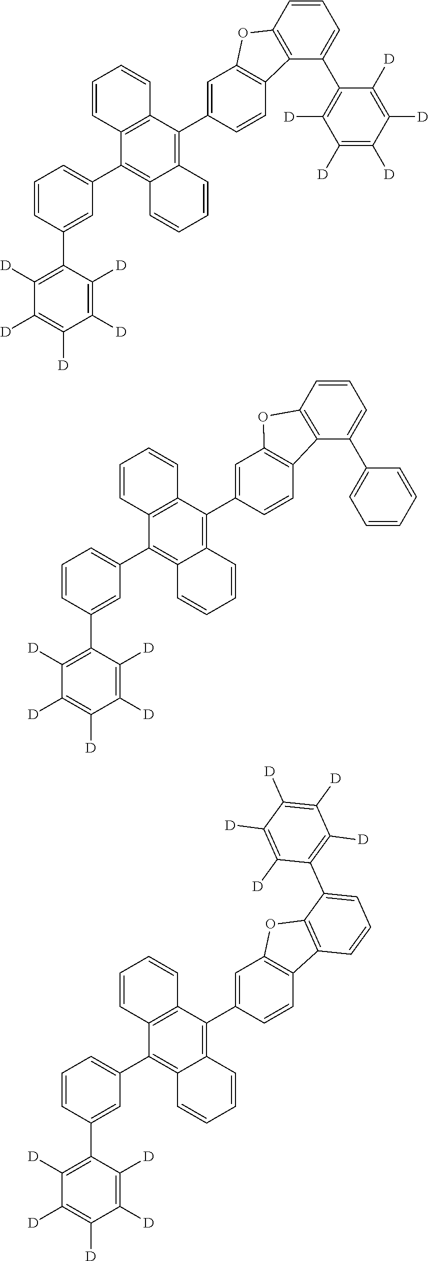 [SMILES: [2H]c1c([2H])c([2H])c(-c2cccc(-c3c4ccccc4c(-c4ccc5c(c4)oc4cccc(-c6c([2H])c([2H])c([2H])c([2H])c6[2H])c45)c4ccccc34)c2)c([2H])c1[2H] ; [2H]c1c([2H])c([2H])c(-c2cccc(-c3c4ccccc4c(-c4ccc5c(c4)oc4cccc(-c6ccccc6)c45)c4ccccc34)c2)c([2H])c1[2H] ; [2H]c1c([2H])c([2H])c(-c2cccc(-c3c4ccccc4c(-c4ccc5c(c4)oc4c(-c6c([2H])c([2H])c([2H])c([2H])c6[2H])cccc45)c4ccccc34)c2)c([2H])c1[2H]]

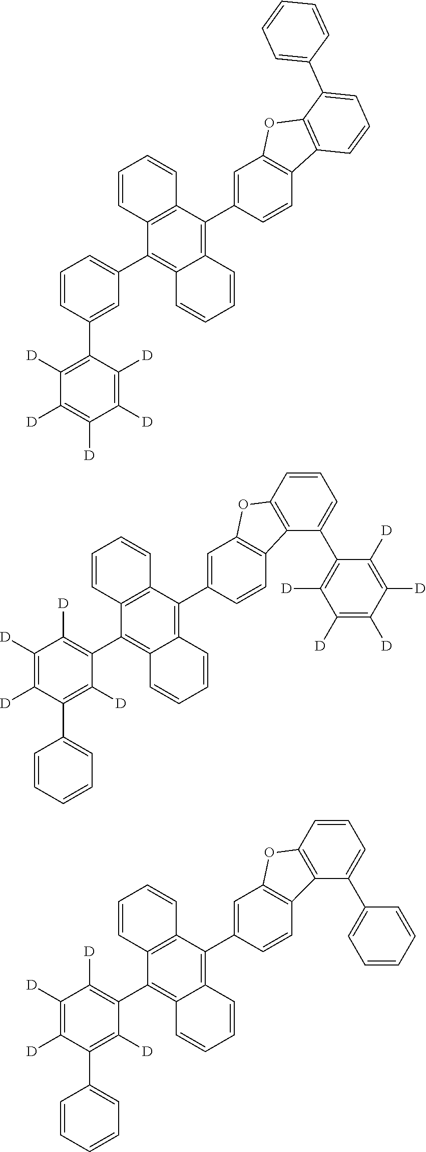 [SMILES: [2H]c1c([2H])c([2H])c(-c2cccc(-c3c4ccccc4c(-c4ccc5c(c4)oc4c(-c6ccccc6)cccc45)c4ccccc34)c2)c([2H])c1[2H] ; [2H]c1c([2H])c([2H])c(-c2cccc3oc4cc(-c5c6ccccc6c(-c6c([2H])c([2H])c([2H])c(-c7ccccc7)c6[2H])c6ccccc56)ccc4c23)c([2H])c1[2H] ; [2H]c1c([2H])c(-c2ccccc2)c([2H])c(-c2c3ccccc3c(-c3ccc4c(c3)oc3cccc(-c5ccccc5)c34)c3ccccc23)c1[2H]]

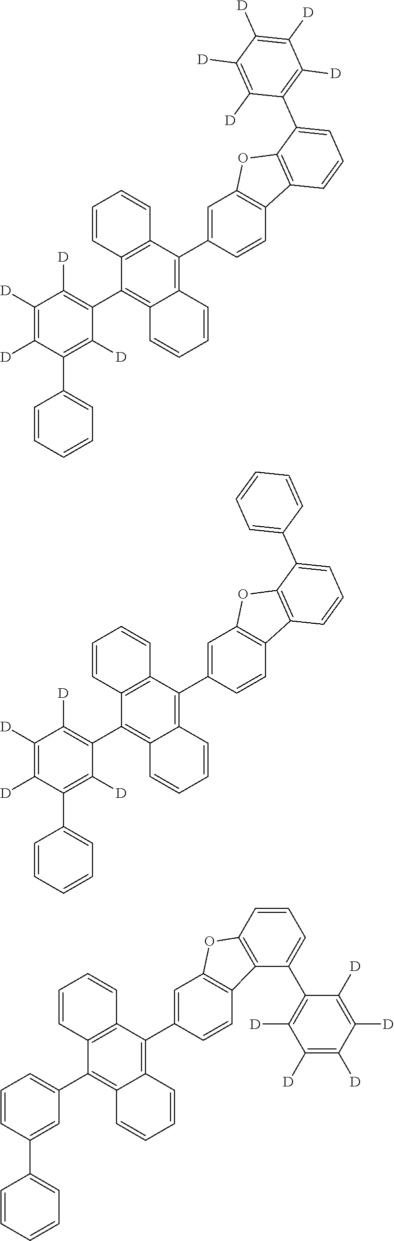 [SMILES: [2H]c1c([2H])c([2H])c(-c2cccc3c2oc2cc(-c4c5ccccc5c(-c5c([2H])c([2H])c([2H])c(-c6ccccc6)c5[2H])c5ccccc45)ccc23)c([2H])c1[2H] ; [2H]c1c([2H])c(-c2ccccc2)c([2H])c(-c2c3ccccc3c(-c3ccc4c(c3)oc3c(-c5ccccc5)cccc34)c3ccccc23)c1[2H] ; [2H]c1c([2H])c([2H])c(-c2cccc3oc4cc(-c5c6ccccc6c(-c6cccc(-c7ccccc7)c6)c6ccccc56)ccc4c23)c([2H])c1[2H]]

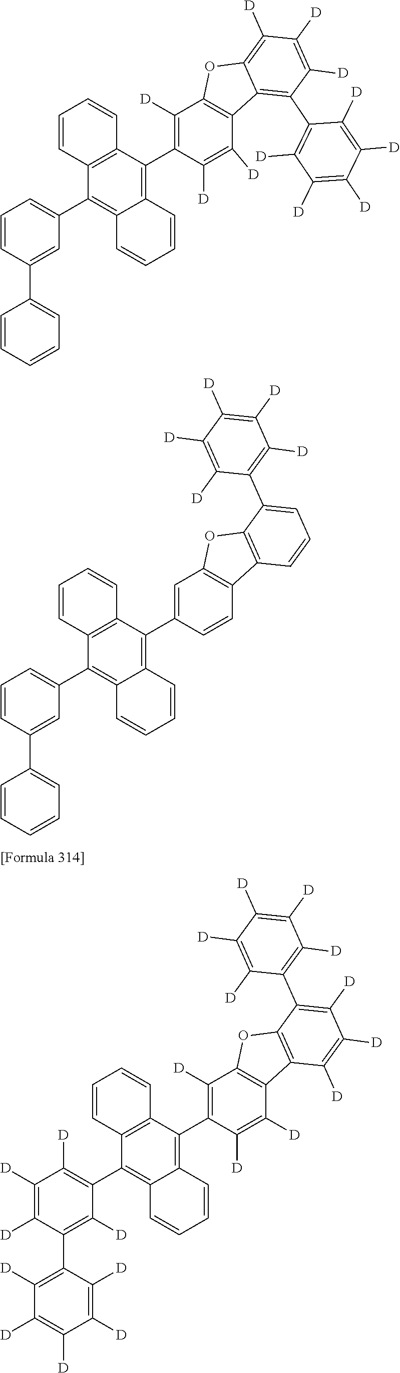 [SMILES: [2H]c1c([2H])c([2H])c(-c2c([2H])c([2H])c([2H])c3oc4c([2H])c(-c5c6ccccc6c(-c6cccc(-c7ccccc7)c6)c6ccccc56)c([2H])c([2H])c4c23)c([2H])c1[2H] ; [2H]c1c([2H])c([2H])c(-c2cccc3c2oc2cc(-c4c5ccccc5c(-c5cccc(-c6ccccc6)c5)c5ccccc45)ccc23)c([2H])c1[2H] ; [2H]c1c([2H])c([2H])c(-c2c([2H])c([2H])c([2H])c(-c3c4ccccc4c(-c4c([2H])c([2H])c5c(oc6c(-c7c([2H])c([2H])c([2H])c([2H])c7[2H])c([2H])c([2H])c([2H])c65)c4[2H])c4ccccc34)c2[2H])c([2H])c1[2H]]

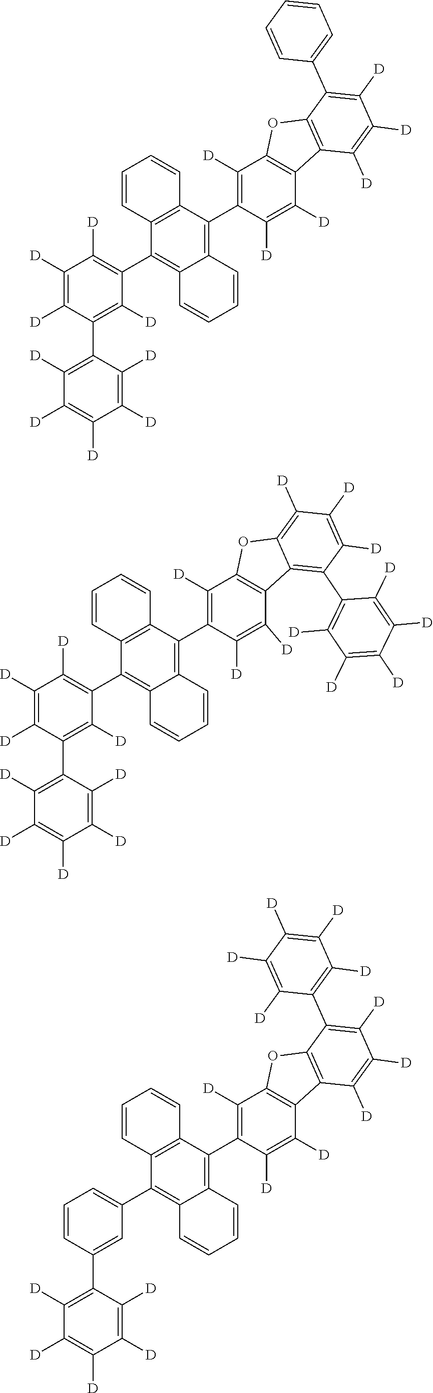

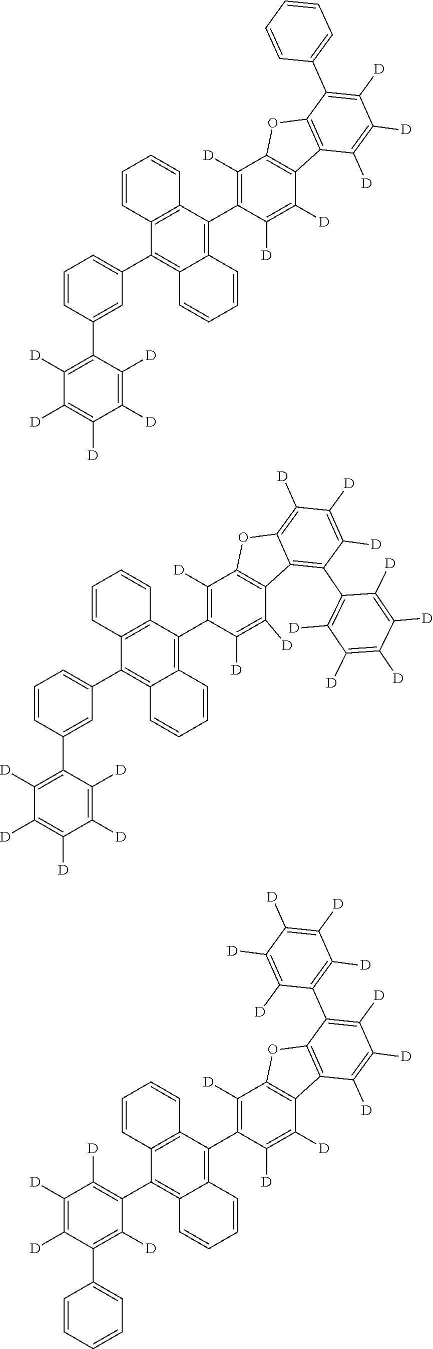 [SMILES: [2H]c1c([2H])c([2H])c(-c2cccc(-c3c4ccccc4c(-c4c([2H])c([2H])c5c(oc6c(-c7ccccc7)c([2H])c([2H])c([2H])c65)c4[2H])c4ccccc34)c2)c([2H])c1[2H] ; [2H]c1c([2H])c([2H])c(-c2cccc(-c3c4ccccc4c(-c4c([2H])c([2H])c5c(oc6c([2H])c([2H])c([2H])c(-c7c([2H])c([2H])c([2H])c([2H])c7[2H])c65)c4[2H])c4ccccc34)c2)c([2H])c1[2H] ; [2H]c1c([2H])c([2H])c(-c2c([2H])c([2H])c([2H])c3c2oc2c([2H])c(-c4c5ccccc5c(-c5c([2H])c([2H])c([2H])c(-c6ccccc6)c5[2H])c5ccccc45)c([2H])c([2H])c23)c([2H])c1[2H]]

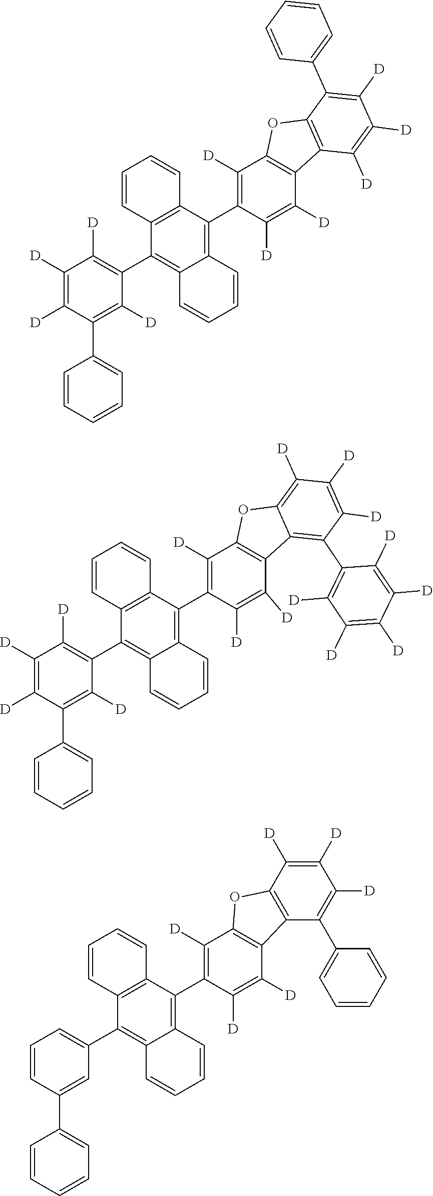 [SMILES: [2H]c1c([2H])c(-c2ccccc2)c([2H])c(-c2c3ccccc3c(-c3c([2H])c([2H])c4c(oc5c(-c6ccccc6)c([2H])c([2H])c([2H])c54)c3[2H])c3ccccc23)c1[2H] ; [2H]c1c([2H])c([2H])c(-c2c([2H])c([2H])c([2H])c3oc4c([2H])c(-c5c6ccccc6c(-c6c([2H])c([2H])c([2H])c(-c7ccccc7)c6[2H])c6ccccc56)c([2H])c([2H])c4c23)c([2H])c1[2H] ; [2H]c1c([2H])c(-c2ccccc2)c2c(oc3c([2H])c(-c4c5ccccc5c(-c5cccc(-c6ccccc6)c5)c5ccccc45)c([2H])c([2H])c32)c1[2H]]

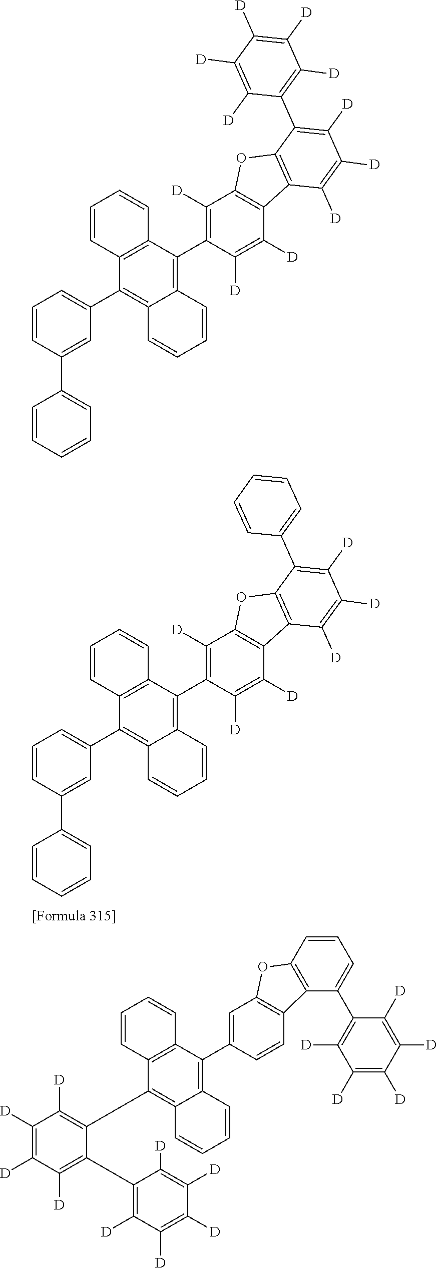 [SMILES: [2H]c1c([2H])c([2H])c(-c2c([2H])c([2H])c([2H])c3c2oc2c([2H])c(-c4c5ccccc5c(-c5cccc(-c6ccccc6)c5)c5ccccc45)c([2H])c([2H])c23)c([2H])c1[2H] ; [2H]c1c([2H])c([2H])c2c(oc3c([2H])c(-c4c5ccccc5c(-c5cccc(-c6ccccc6)c5)c5ccccc45)c([2H])c([2H])c32)c1-c1ccccc1 ; [2H]c1c([2H])c([2H])c(-c2c([2H])c([2H])c([2H])c([2H])c2-c2c3ccccc3c(-c3ccc4c(c3)oc3cccc(-c5c([2H])c([2H])c([2H])c([2H])c5[2H])c34)c3ccccc23)c([2H])c1[2H]]

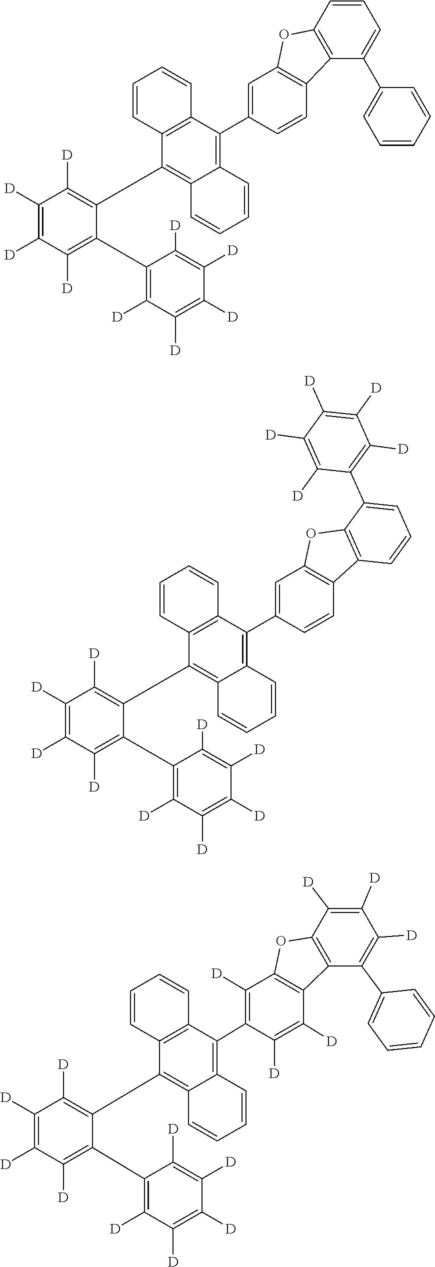 [SMILES: [2H]c1c([2H])c([2H])c(-c2c([2H])c([2H])c([2H])c([2H])c2-c2c3ccccc3c(-c3ccc4c(c3)oc3cccc(-c5ccccc5)c34)c3ccccc23)c([2H])c1[2H] ; [2H]c1c([2H])c([2H])c(-c2c([2H])c([2H])c([2H])c([2H])c2-c2c3ccccc3c(-c3ccc4c(c3)oc3c(-c5c([2H])c([2H])c([2H])c([2H])c5[2H])cccc34)c3ccccc23)c([2H])c1[2H] ; [2H]c1c([2H])c([2H])c(-c2c([2H])c([2H])c([2H])c([2H])c2-c2c3ccccc3c(-c3c([2H])c([2H])c4c(oc5c([2H])c([2H])c([2H])c(-c6ccccc6)c54)c3[2H])c3ccccc23)c([2H])c1[2H]]

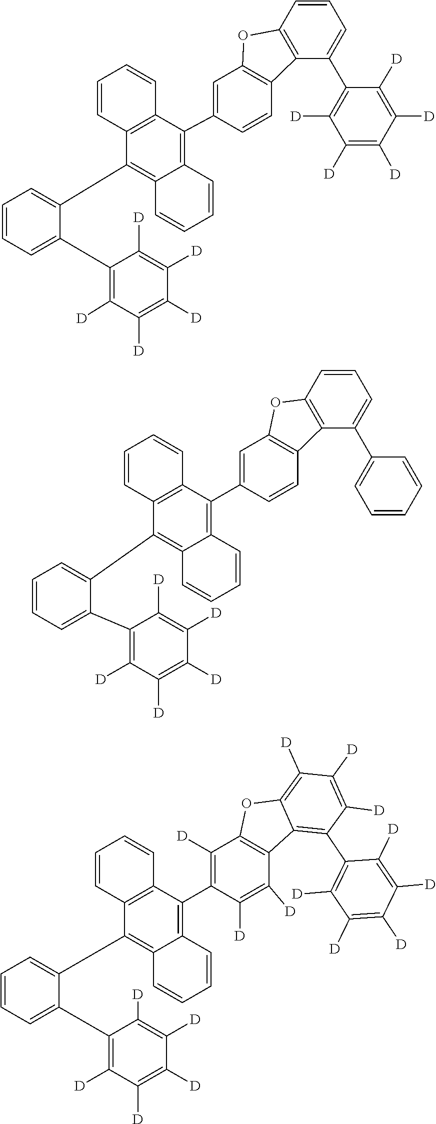 [SMILES: [2H]c1c([2H])c([2H])c(-c2ccccc2-c2c3ccccc3c(-c3ccc4c(c3)oc3cccc(-c5c([2H])c([2H])c([2H])c([2H])c5[2H])c34)c3ccccc23)c([2H])c1[2H] ; [2H]c1c([2H])c([2H])c(-c2ccccc2-c2c3ccccc3c(-c3ccc4c(c3)oc3cccc(-c5ccccc5)c34)c3ccccc23)c([2H])c1[2H] ; [2H]c1c([2H])c([2H])c(-c2ccccc2-c2c3ccccc3c(-c3c([2H])c([2H])c4c(oc5c([2H])c([2H])c([2H])c(-c6c([2H])c([2H])c([2H])c([2H])c6[2H])c54)c3[2H])c3ccccc23)c([2H])c1[2H]]

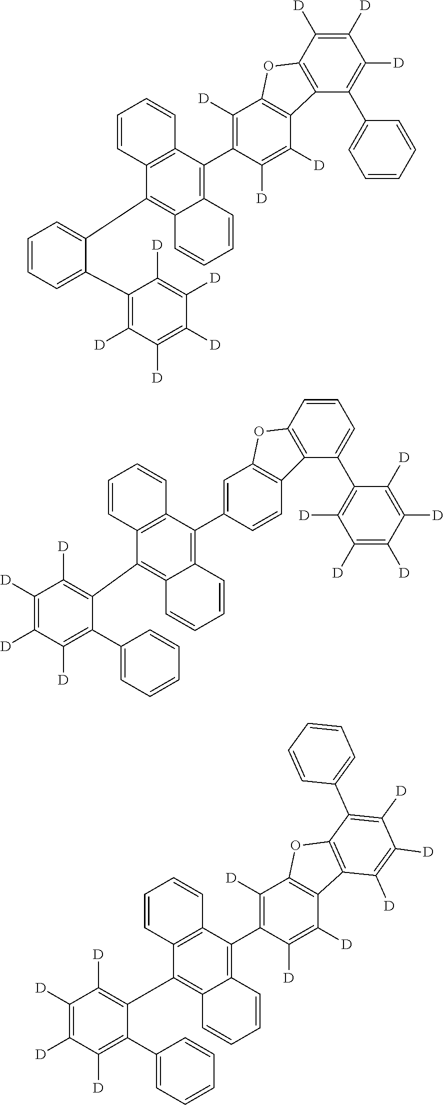 [SMILES: [2H]c1c([2H])c([2H])c(-c2ccccc2-c2c3ccccc3c(-c3c([2H])c([2H])c4c(oc5c([2H])c([2H])c([2H])c(-c6ccccc6)c54)c3[2H])c3ccccc23)c([2H])c1[2H] ; [2H]c1c([2H])c([2H])c(-c2cccc3oc4cc(-c5c6ccccc6c(-c6c([2H])c([2H])c([2H])c([2H])c6-c6ccccc6)c6ccccc56)ccc4c23)c([2H])c1[2H] ; [2H]c1c([2H])c([2H])c(-c2c3ccccc3c(-c3c([2H])c([2H])c4c(oc5c(-c6ccccc6)c([2H])c([2H])c([2H])c54)c3[2H])c3ccccc23)c(-c2ccccc2)c1[2H]]

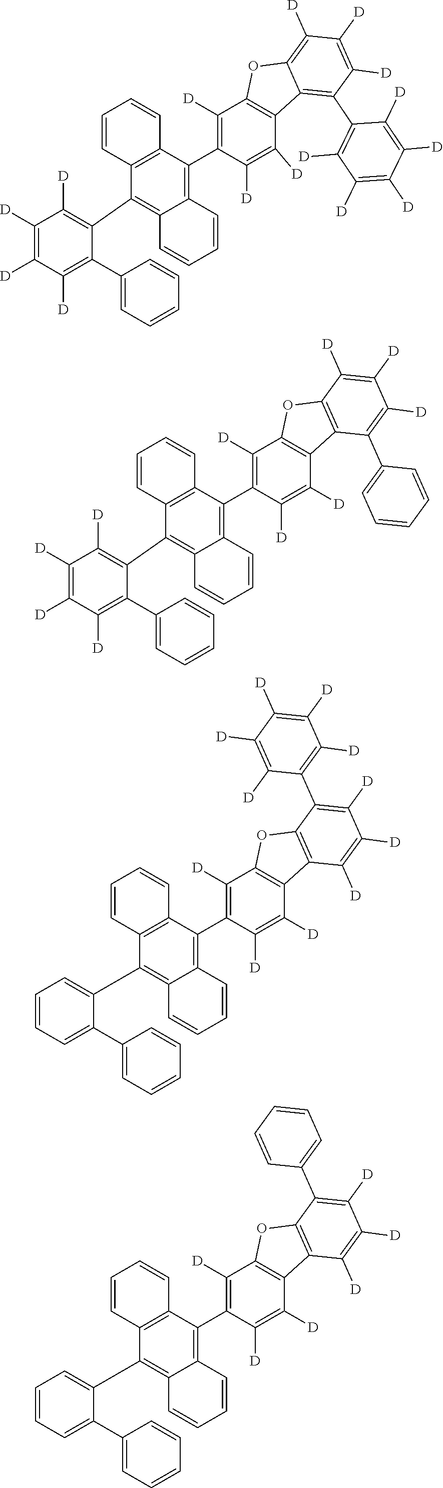

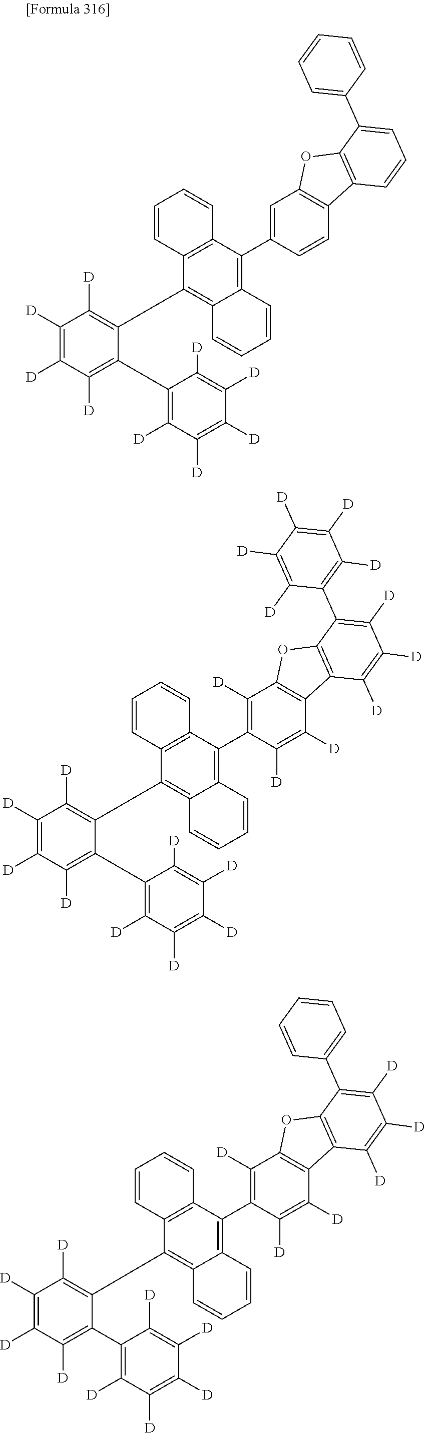 [SMILES: [2H]c1c([2H])c([2H])c(-c2c([2H])c([2H])c([2H])c([2H])c2-c2c3ccccc3c(-c3ccc4c(c3)oc3c(-c5ccccc5)cccc34)c3ccccc23)c([2H])c1[2H] ; [2H]c1c([2H])c([2H])c(-c2c([2H])c([2H])c([2H])c([2H])c2-c2c3ccccc3c(-c3c([2H])c([2H])c4c(oc5c(-c6c([2H])c([2H])c([2H])c([2H])c6[2H])c([2H])c([2H])c([2H])c54)c3[2H])c3ccccc23)c([2H])c1[2H] ; [2H]c1c([2H])c([2H])c(-c2c([2H])c([2H])c([2H])c([2H])c2-c2c3ccccc3c(-c3c([2H])c([2H])c4c(oc5c(-c6ccccc6)c([2H])c([2H])c([2H])c54)c3[2H])c3ccccc23)c([2H])c1[2H]]

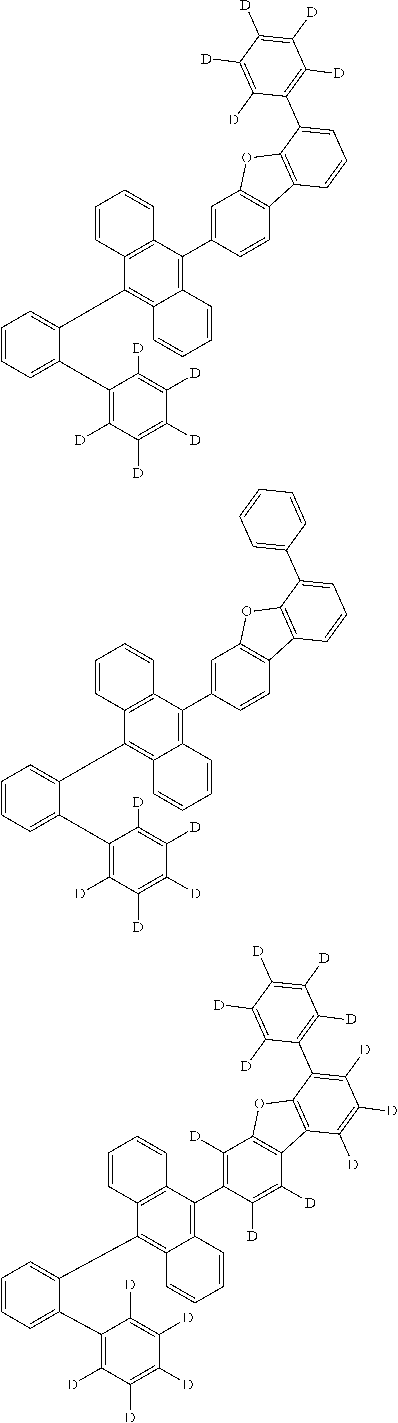 [SMILES: [2H]c1c([2H])c([2H])c(-c2ccccc2-c2c3ccccc3c(-c3ccc4c(c3)oc3c(-c5c([2H])c([2H])c([2H])c([2H])c5[2H])cccc34)c3ccccc23)c([2H])c1[2H] ; [2H]c1c([2H])c([2H])c(-c2ccccc2-c2c3ccccc3c(-c3ccc4c(c3)oc3c(-c5ccccc5)cccc34)c3ccccc23)c([2H])c1[2H] ; [2H]c1c([2H])c([2H])c(-c2ccccc2-c2c3ccccc3c(-c3c([2H])c([2H])c4c(oc5c(-c6c([2H])c([2H])c([2H])c([2H])c6[2H])c([2H])c([2H])c([2H])c54)c3[2H])c3ccccc23)c([2H])c1[2H]]

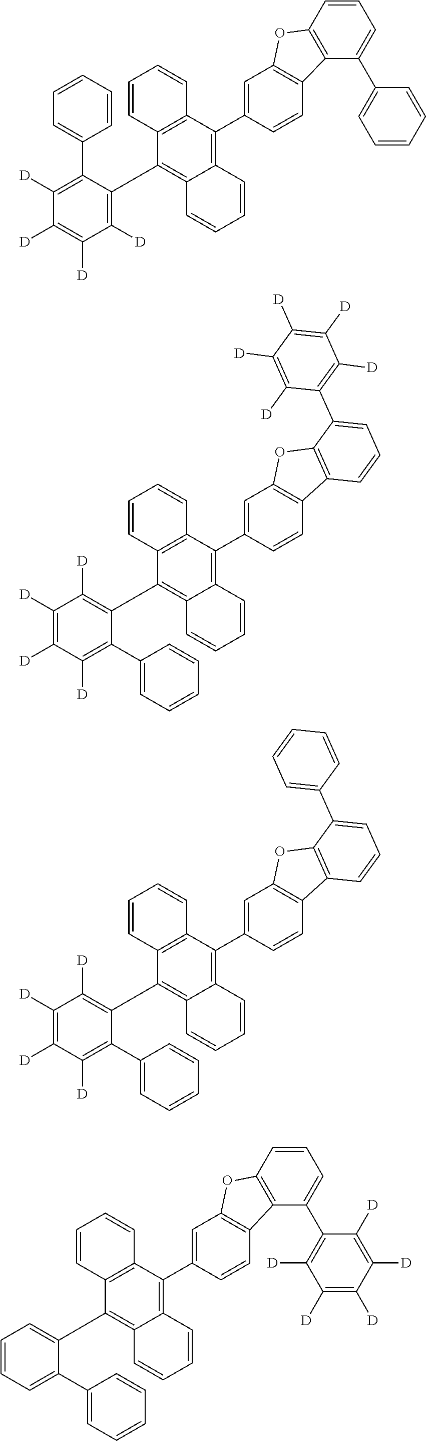 [SMILES: [2H]c1c([2H])c([2H])c(-c2c3ccccc3c(-c3ccc4c(c3)oc3cccc(-c5ccccc5)c34)c3ccccc23)c(-c2ccccc2)c1[2H] ; [2H]c1c([2H])c([2H])c(-c2cccc3c2oc2cc(-c4c5ccccc5c(-c5c([2H])c([2H])c([2H])c([2H])c5-c5ccccc5)c5ccccc45)ccc23)c([2H])c1[2H] ; [2H]c1c([2H])c([2H])c(-c2c3ccccc3c(-c3ccc4c(c3)oc3c(-c5ccccc5)cccc34)c3ccccc23)c(-c2ccccc2)c1[2H] ; [2H]c1c([2H])c([2H])c(-c2cccc3oc4cc(-c5c6ccccc6c(-c6ccccc6-c6ccccc6)c6ccccc56)ccc4c23)c([2H])c1[2H]]

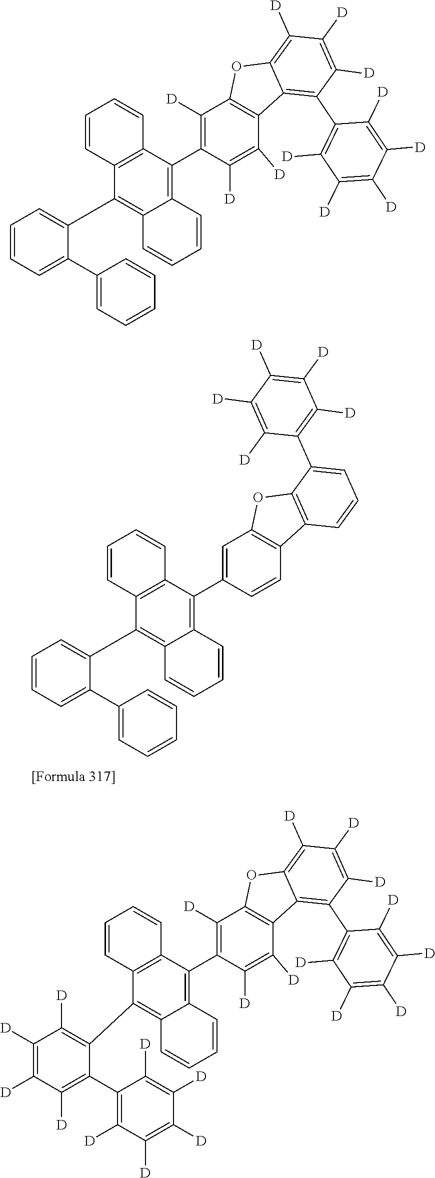 [SMILES: [2H]c1c([2H])c([2H])c(-c2c([2H])c([2H])c([2H])c3oc4c([2H])c(-c5c6ccccc6c(-c6ccccc6-c6ccccc6)c6ccccc56)c([2H])c([2H])c4c23)c([2H])c1[2H] ; [2H]c1c([2H])c([2H])c(-c2cccc3c2oc2cc(-c4c5ccccc5c(-c5ccccc5-c5ccccc5)c5ccccc45)ccc23)c([2H])c1[2H] ; [2H]c1c([2H])c([2H])c(-c2c([2H])c([2H])c([2H])c([2H])c2-c2c3ccccc3c(-c3c([2H])c([2H])c4c(oc5c([2H])c([2H])c([2H])c(-c6c([2H])c([2H])c([2H])c([2H])c6[2H])c54)c3[2H])c3ccccc23)c([2H])c1[2H]]

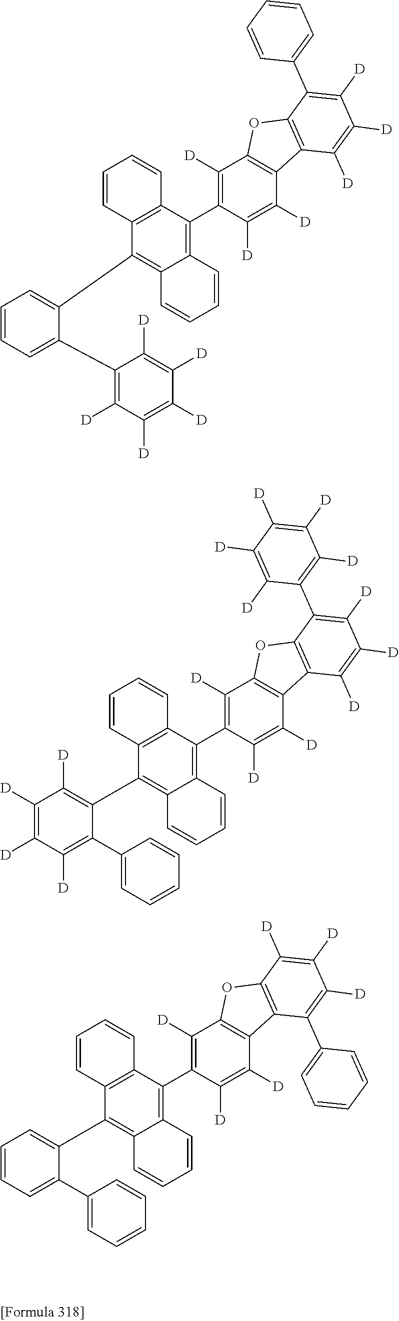 [SMILES: [2H]c1c([2H])c([2H])c(-c2ccccc2-c2c3ccccc3c(-c3c([2H])c([2H])c4c(oc5c(-c6ccccc6)c([2H])c([2H])c([2H])c54)c3[2H])c3ccccc23)c([2H])c1[2H] ; [2H]c1c([2H])c([2H])c(-c2c([2H])c([2H])c([2H])c3c2oc2c([2H])c(-c4c5ccccc5c(-c5c([2H])c([2H])c([2H])c([2H])c5-c5ccccc5)c5ccccc45)c([2H])c([2H])c23)c([2H])c1[2H] ; [2H]c1c([2H])c(-c2ccccc2)c2c(oc3c([2H])c(-c4c5ccccc5c(-c5ccccc5-c5ccccc5)c5ccccc45)c([2H])c([2H])c32)c1[2H]]

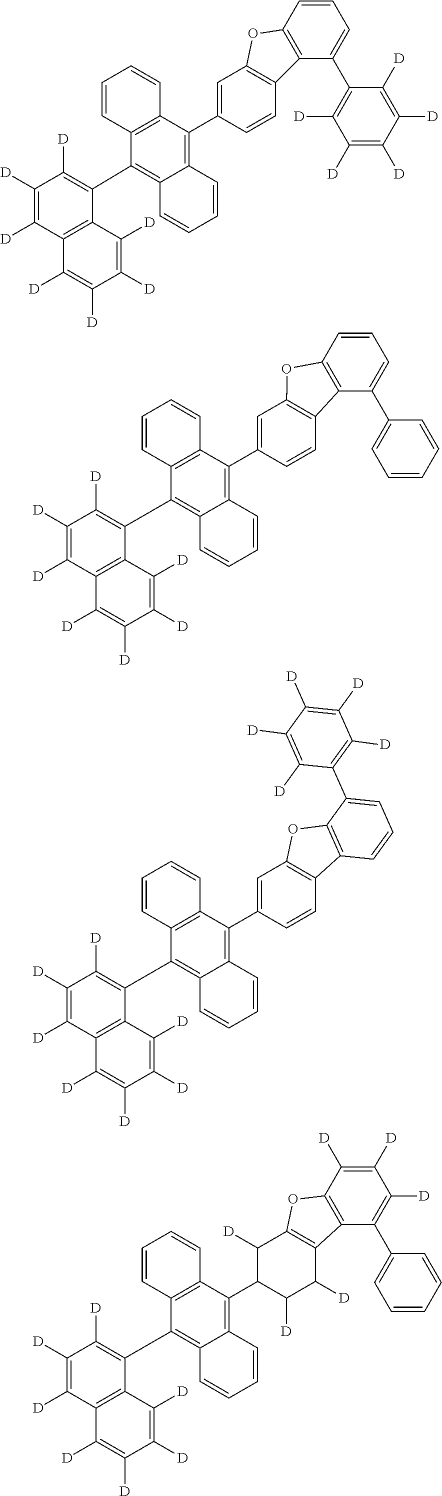

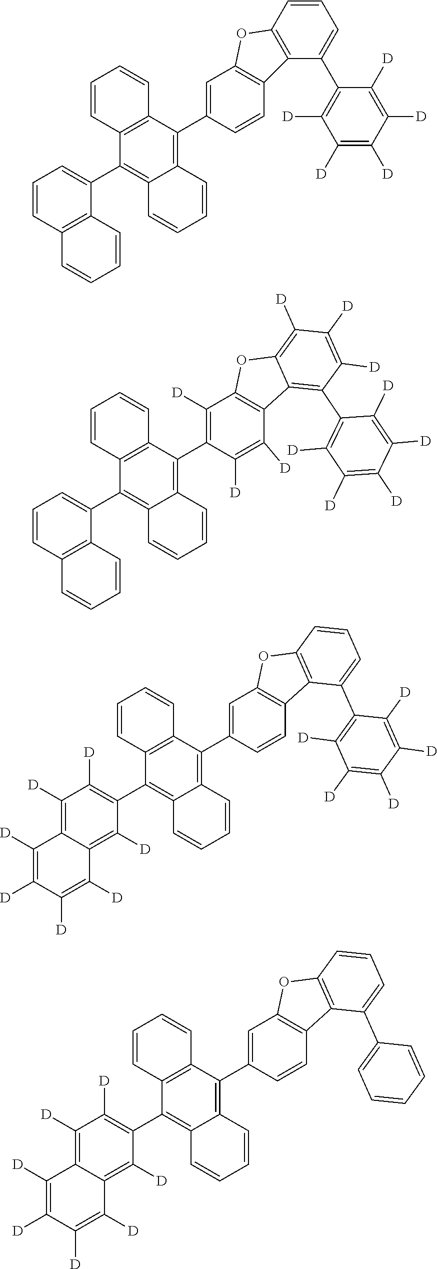 [SMILES: [2H]c1c([2H])c([2H])c(-c2cccc3oc4cc(-c5c6ccccc6c(-c6cccc7ccccc67)c6ccccc56)ccc4c23)c([2H])c1[2H] ; [2H]c1c([2H])c([2H])c(-c2c([2H])c([2H])c([2H])c3oc4c([2H])c(-c5c6ccccc6c(-c6cccc7ccccc67)c6ccccc56)c([2H])c([2H])c4c23)c([2H])c1[2H] ; [2H]c1c([2H])c([2H])c(-c2cccc3oc4cc(-c5c6ccccc6c(-c6c([2H])c([2H])c7c([2H])c([2H])c([2H])c([2H])c7c6[2H])c6ccccc56)ccc4c23)c([2H])c1[2H] ; [2H]c1c([2H])c([2H])c2c([2H])c(-c3c4ccccc4c(-c4ccc5c(c4)oc4cccc(-c6ccccc6)c45)c4ccccc34)c([2H])c([2H])c2c1[2H]]

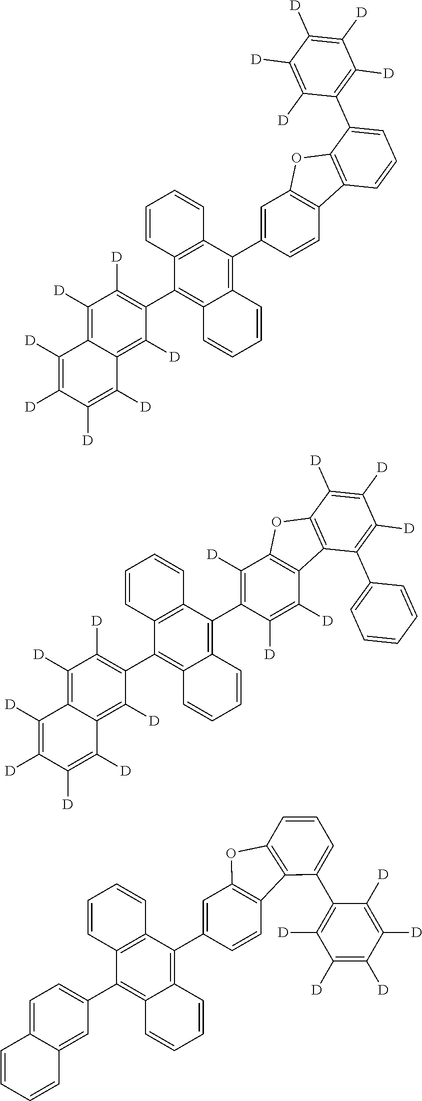 [SMILES: [2H]c1c([2H])c([2H])c(-c2cccc3c2oc2cc(-c4c5ccccc5c(-c5c([2H])c([2H])c6c([2H])c([2H])c([2H])c([2H])c6c5[2H])c5ccccc45)ccc23)c([2H])c1[2H] ; [2H]c1c([2H])c(-c2ccccc2)c2c(oc3c([2H])c(-c4c5ccccc5c(-c5c([2H])c([2H])c6c([2H])c([2H])c([2H])c([2H])c6c5[2H])c5ccccc45)c([2H])c([2H])c32)c1[2H] ; [2H]c1c([2H])c([2H])c(-c2cccc3oc4cc(-c5c6ccccc6c(-c6ccc7ccccc7c6)c6ccccc56)ccc4c23)c([2H])c1[2H]]

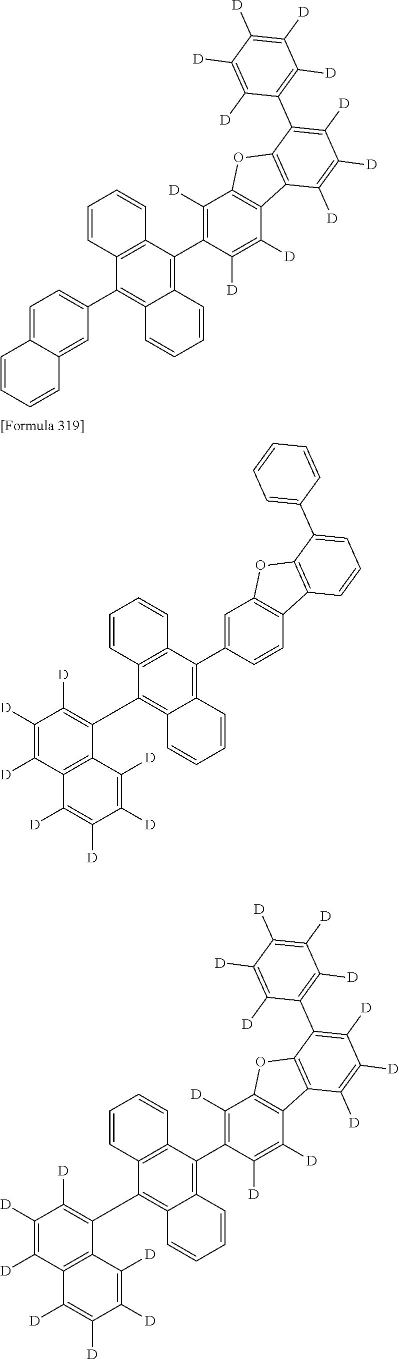 [SMILES: [2H]c1c([2H])c([2H])c(-c2c([2H])c([2H])c([2H])c3c2oc2c([2H])c(-c4c5ccccc5c(-c5ccc6ccccc6c5)c5ccccc45)c([2H])c([2H])c23)c([2H])c1[2H] ; [2H]c1c([2H])c([2H])c2c(-c3c4ccccc4c(-c4ccc5c(c4)oc4c(-c6ccccc6)cccc45)c4ccccc34)c([2H])c([2H])c([2H])c2c1[2H] ; [2H]c1c([2H])c([2H])c(-c2c([2H])c([2H])c([2H])c3c2oc2c([2H])c(-c4c5ccccc5c(-c5c([2H])c([2H])c([2H])c6c([2H])c([2H])c([2H])c([2H])c56)c5ccccc45)c([2H])c([2H])c23)c([2H])c1[2H]]

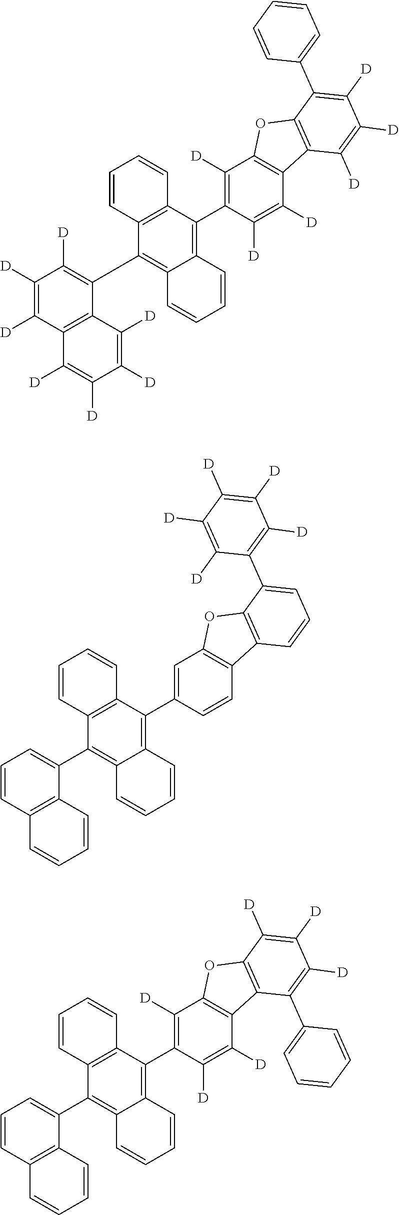 [SMILES: [2H]c1c([2H])c([2H])c2c(oc3c([2H])c(-c4c5ccccc5c(-c5c([2H])c([2H])c([2H])c6c([2H])c([2H])c([2H])c([2H])c56)c5ccccc45)c([2H])c([2H])c32)c1-c1ccccc1 ; [2H]c1c([2H])c([2H])c(-c2cccc3c2oc2cc(-c4c5ccccc5c(-c5cccc6ccccc56)c5ccccc45)ccc23)c([2H])c1[2H] ; [2H]c1c([2H])c(-c2ccccc2)c2c(oc3c([2H])c(-c4c5ccccc5c(-c5cccc6ccccc56)c5ccccc45)c([2H])c([2H])c32)c1[2H]]

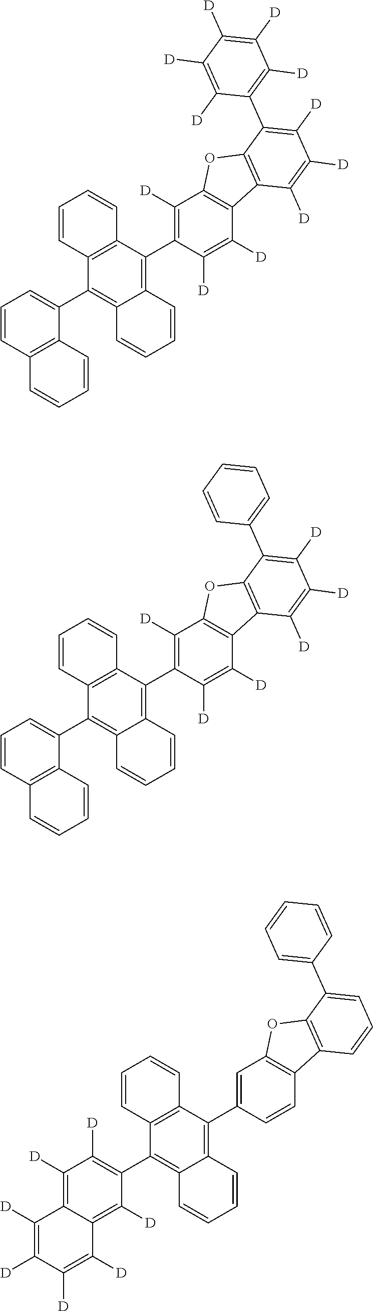 [SMILES: [2H]c1c([2H])c([2H])c(-c2c([2H])c([2H])c([2H])c3c2oc2c([2H])c(-c4c5ccccc5c(-c5cccc6ccccc56)c5ccccc45)c([2H])c([2H])c23)c([2H])c1[2H] ; [2H]c1c([2H])c([2H])c2c(oc3c([2H])c(-c4c5ccccc5c(-c5cccc6ccccc56)c5ccccc45)c([2H])c([2H])c32)c1-c1ccccc1 ; [2H]c1c([2H])c([2H])c2c([2H])c(-c3c4ccccc4c(-c4ccc5c(c4)oc4c(-c6ccccc6)cccc45)c4ccccc34)c([2H])c([2H])c2c1[2H]]

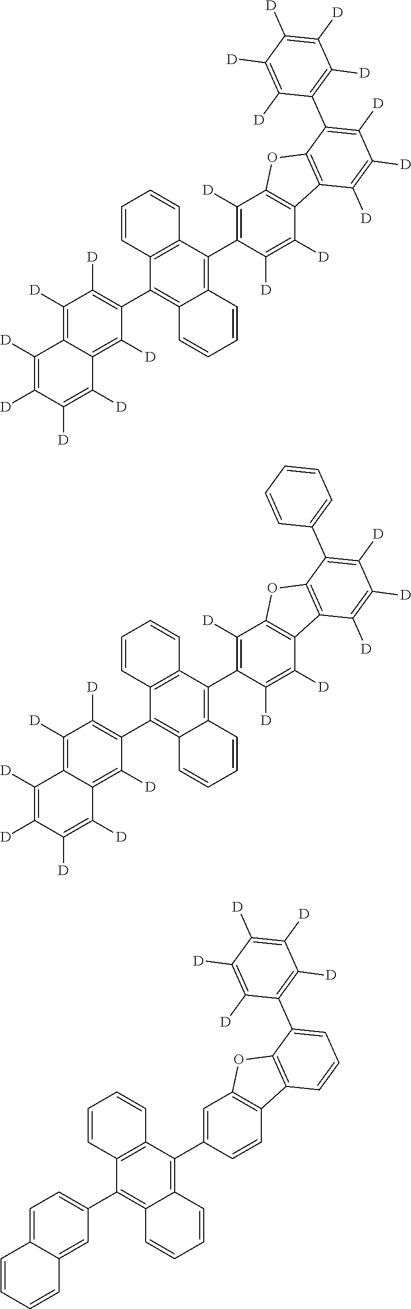 [SMILES: [2H]c1c([2H])c([2H])c(-c2c([2H])c([2H])c([2H])c3c2oc2c([2H])c(-c4c5ccccc5c(-c5c([2H])c([2H])c6c([2H])c([2H])c([2H])c([2H])c6c5[2H])c5ccccc45)c([2H])c([2H])c23)c([2H])c1[2H] ; [2H]c1c([2H])c([2H])c2c(oc3c([2H])c(-c4c5ccccc5c(-c5c([2H])c([2H])c6c([2H])c([2H])c([2H])c([2H])c6c5[2H])c5ccccc45)c([2H])c([2H])c32)c1-c1ccccc1 ; [2H]c1c([2H])c([2H])c(-c2cccc3c2oc2cc(-c4c5ccccc5c(-c5ccc6ccccc6c5)c5ccccc45)ccc23)c([2H])c1[2H]]

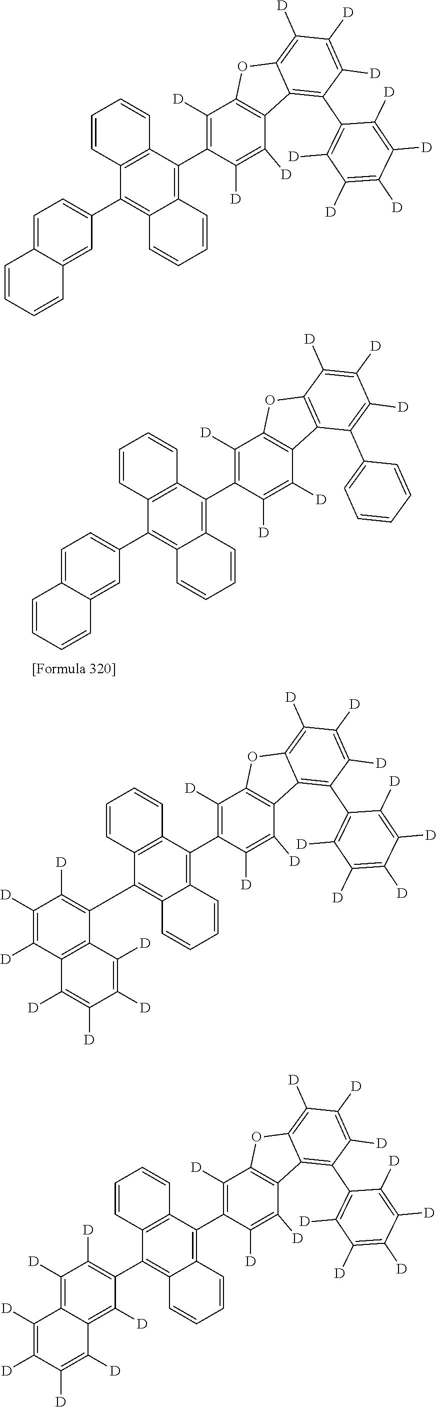

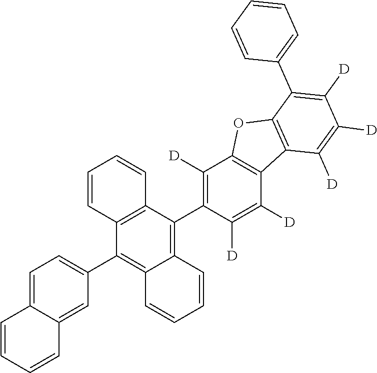 [SMILES: [2H]c1c([2H])c([2H])c2c(oc3c([2H])c(-c4c5ccccc5c(-c5ccc6ccccc6c5)c5ccccc45)c([2H])c([2H])c32)c1-c1ccccc1]

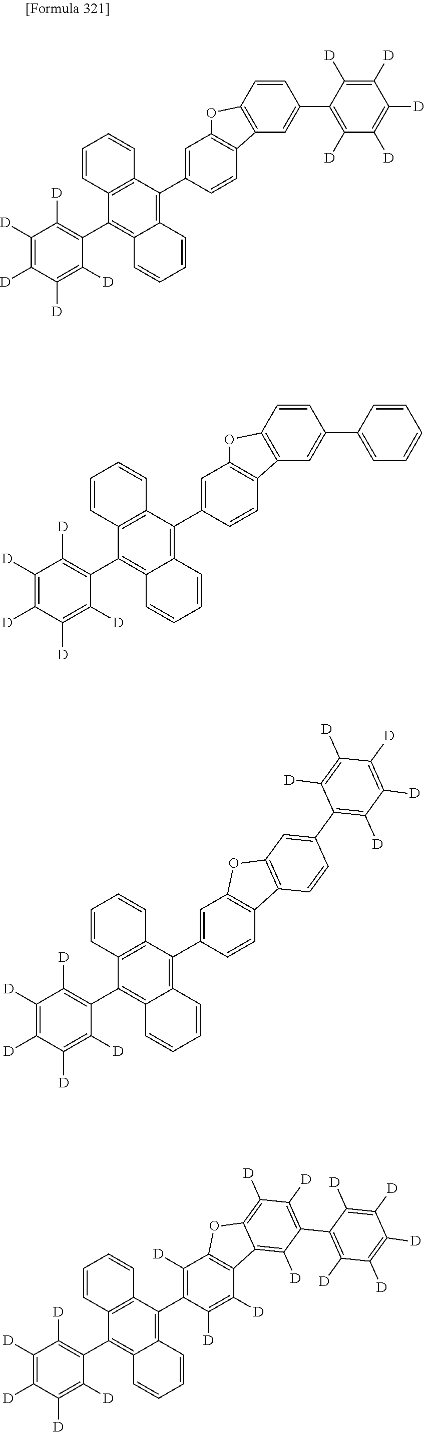 [SMILES: [2H]c1c([2H])c([2H])c(-c2ccc3oc4cc(-c5c6ccccc6c(-c6c([2H])c([2H])c([2H])c([2H])c6[2H])c6ccccc56)ccc4c3c2)c([2H])c1[2H] ; [2H]c1c([2H])c([2H])c(-c2c3ccccc3c(-c3ccc4c(c3)oc3ccc(-c5ccccc5)cc34)c3ccccc23)c([2H])c1[2H] ; [2H]c1c([2H])c([2H])c(-c2ccc3c(c2)oc2cc(-c4c5ccccc5c(-c5c([2H])c([2H])c([2H])c([2H])c5[2H])c5ccccc45)ccc23)c([2H])c1[2H] ; [2H]c1c([2H])c([2H])c(-c2c([2H])c([2H])c3oc4c([2H])c(-c5c6ccccc6c(-c6c([2H])c([2H])c([2H])c([2H])c6[2H])c6ccccc56)c([2H])c([2H])c4c3c2[2H])c([2H])c1[2H]]

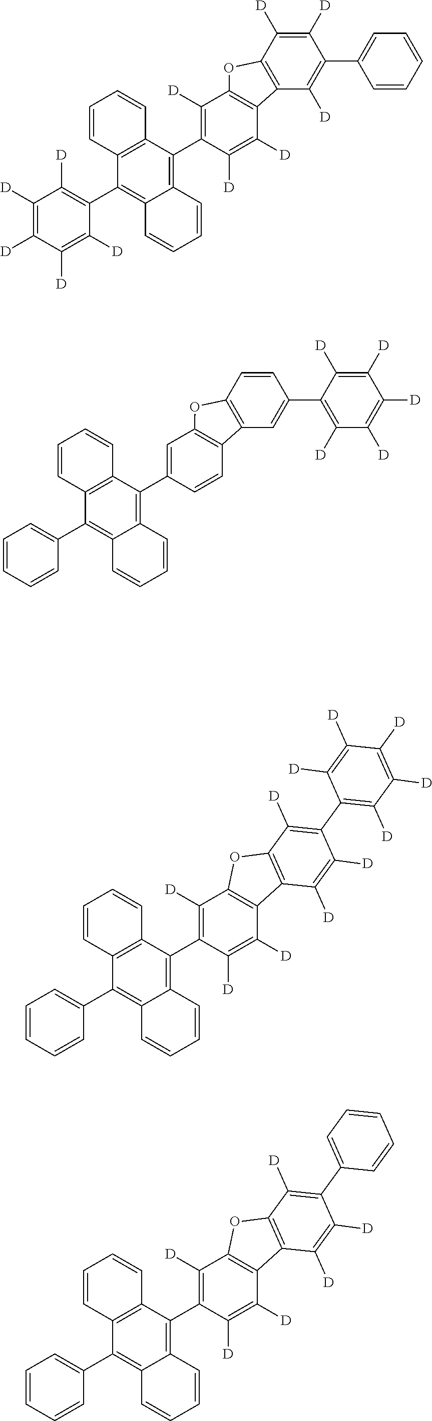 [SMILES: [2H]c1c([2H])c([2H])c(-c2c3ccccc3c(-c3c([2H])c([2H])c4c(oc5c([2H])c([2H])c(-c6ccccc6)c([2H])c54)c3[2H])c3ccccc23)c([2H])c1[2H] ; [2H]c1c([2H])c([2H])c(-c2ccc3oc4cc(-c5c6ccccc6c(-c6ccccc6)c6ccccc56)ccc4c3c2)c([2H])c1[2H] ; [2H]c1c([2H])c([2H])c(-c2c([2H])c([2H])c3c(oc4c([2H])c(-c5c6ccccc6c(-c6ccccc6)c6ccccc56)c([2H])c([2H])c43)c2[2H])c([2H])c1[2H] ; [2H]c1c(-c2ccccc2)c([2H])c2oc3c([2H])c(-c4c5ccccc5c(-c5ccccc5)c5ccccc45)c([2H])c([2H])c3c2c1[2H]]

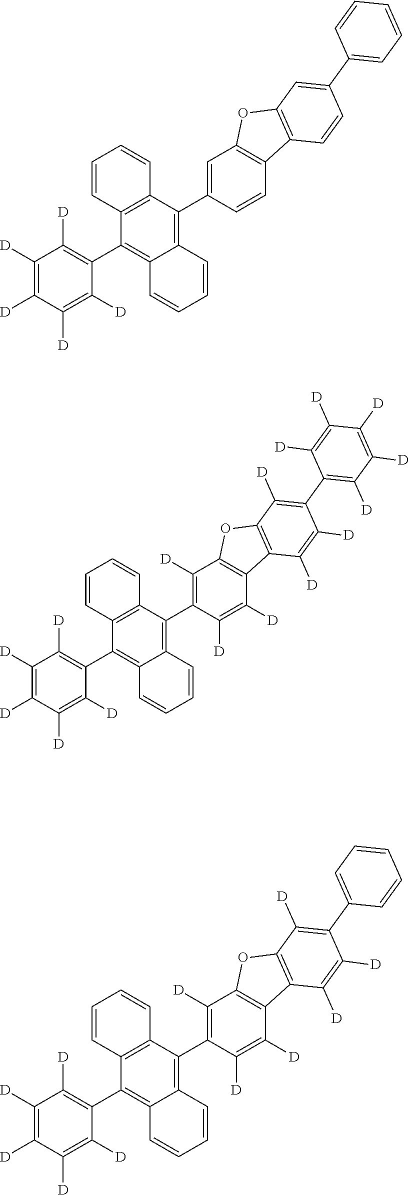 [SMILES: [2H]c1c([2H])c([2H])c(-c2c3ccccc3c(-c3ccc4c(c3)oc3cc(-c5ccccc5)ccc34)c3ccccc23)c([2H])c1[2H] ; [2H]c1c([2H])c([2H])c(-c2c([2H])c([2H])c3c(oc4c([2H])c(-c5c6ccccc6c(-c6c([2H])c([2H])c([2H])c([2H])c6[2H])c6ccccc56)c([2H])c([2H])c43)c2[2H])c([2H])c1[2H] ; [2H]c1c([2H])c([2H])c(-c2c3ccccc3c(-c3c([2H])c([2H])c4c(oc5c([2H])c(-c6ccccc6)c([2H])c([2H])c54)c3[2H])c3ccccc23)c([2H])c1[2H]]

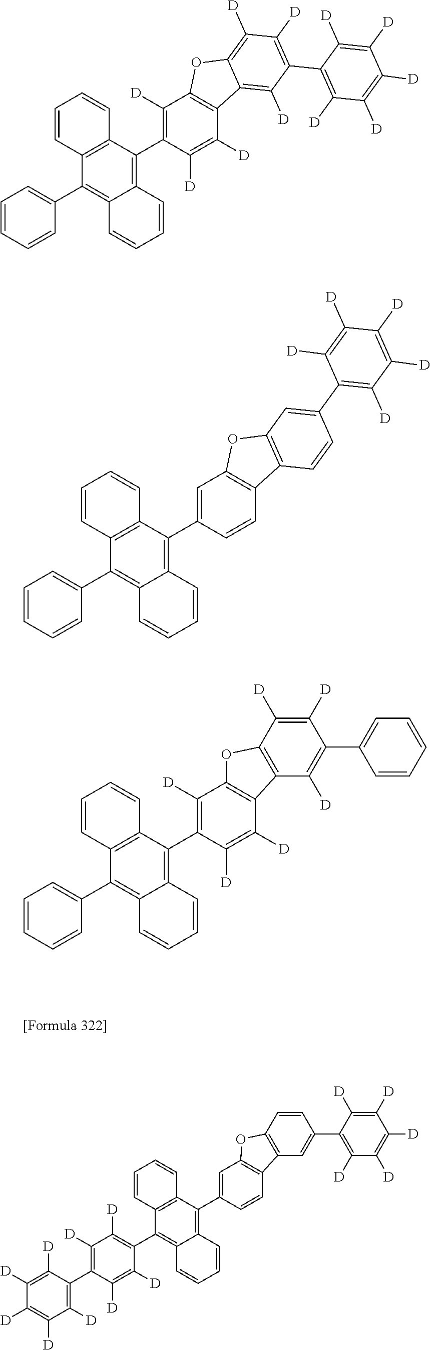 [SMILES: [2H]c1c([2H])c([2H])c(-c2c([2H])c([2H])c3oc4c([2H])c(-c5c6ccccc6c(-c6ccccc6)c6ccccc56)c([2H])c([2H])c4c3c2[2H])c([2H])c1[2H] ; [2H]c1c([2H])c([2H])c(-c2ccc3c(c2)oc2cc(-c4c5ccccc5c(-c5ccccc5)c5ccccc45)ccc23)c([2H])c1[2H] ; [2H]c1c(-c2ccccc2)c([2H])c2c(oc3c([2H])c(-c4c5ccccc5c(-c5ccccc5)c5ccccc45)c([2H])c([2H])c32)c1[2H] ; [2H]c1c([2H])c([2H])c(-c2ccc3oc4cc(-c5c6ccccc6c(-c6c([2H])c([2H])c(-c7c([2H])c([2H])c([2H])c([2H])c7[2H])c([2H])c6[2H])c6ccccc56)ccc4c3c2)c([2H])c1[2H]]

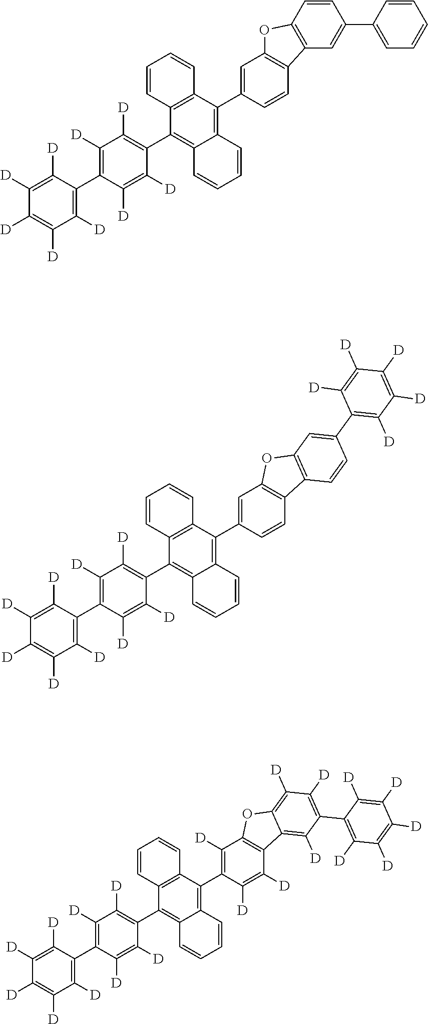 [SMILES: [2H]c1c([2H])c([2H])c(-c2c([2H])c([2H])c(-c3c4ccccc4c(-c4ccc5c(c4)oc4ccc(-c6ccccc6)cc45)c4ccccc34)c([2H])c2[2H])c([2H])c1[2H] ; [2H]c1c([2H])c([2H])c(-c2ccc3c(c2)oc2cc(-c4c5ccccc5c(-c5c([2H])c([2H])c(-c6c([2H])c([2H])c([2H])c([2H])c6[2H])c([2H])c5[2H])c5ccccc45)ccc23)c([2H])c1[2H] ; [2H]c1c([2H])c([2H])c(-c2c([2H])c([2H])c(-c3c4ccccc4c(-c4c([2H])c([2H])c5c(oc6c([2H])c([2H])c(-c7c([2H])c([2H])c([2H])c([2H])c7[2H])c([2H])c65)c4[2H])c4ccccc34)c([2H])c2[2H])c([2H])c1[2H]]

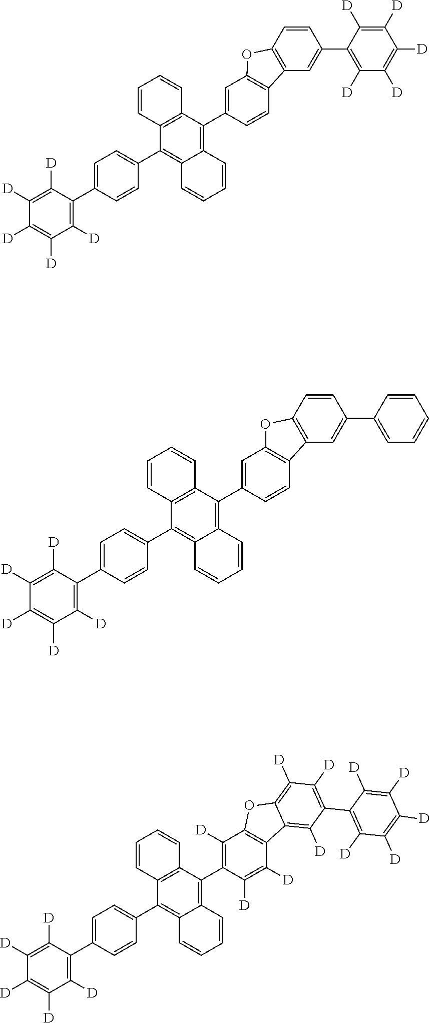 [SMILES: [2H]c1c([2H])c([2H])c(-c2ccc(-c3c4ccccc4c(-c4ccc5c(c4)oc4ccc(-c6c([2H])c([2H])c([2H])c([2H])c6[2H])cc45)c4ccccc34)cc2)c([2H])c1[2H] ; [2H]c1c([2H])c([2H])c(-c2ccc(-c3c4ccccc4c(-c4ccc5c(c4)oc4ccc(-c6ccccc6)cc45)c4ccccc34)cc2)c([2H])c1[2H] ; [2H]c1c([2H])c([2H])c(-c2ccc(-c3c4ccccc4c(-c4c([2H])c([2H])c5c(oc6c([2H])c([2H])c(-c7c([2H])c([2H])c([2H])c([2H])c7[2H])c([2H])c65)c4[2H])c4ccccc34)cc2)c([2H])c1[2H]]

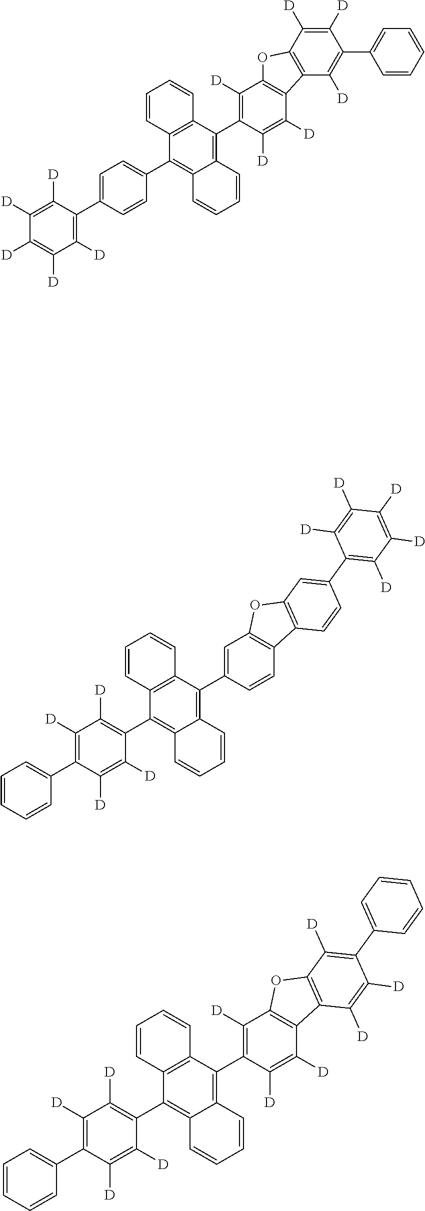 [SMILES: [2H]c1c([2H])c([2H])c(-c2ccc(-c3c4ccccc4c(-c4c([2H])c([2H])c5c(oc6c([2H])c([2H])c(-c7ccccc7)c([2H])c65)c4[2H])c4ccccc34)cc2)c([2H])c1[2H] ; [2H]c1c([2H])c([2H])c(-c2ccc3c(c2)oc2cc(-c4c5ccccc5c(-c5c([2H])c([2H])c(-c6ccccc6)c([2H])c5[2H])c5ccccc45)ccc23)c([2H])c1[2H] ; [2H]c1c([2H])c(-c2c3ccccc3c(-c3c([2H])c([2H])c4c(oc5c([2H])c(-c6ccccc6)c([2H])c([2H])c54)c3[2H])c3ccccc23)c([2H])c([2H])c1-c1ccccc1]

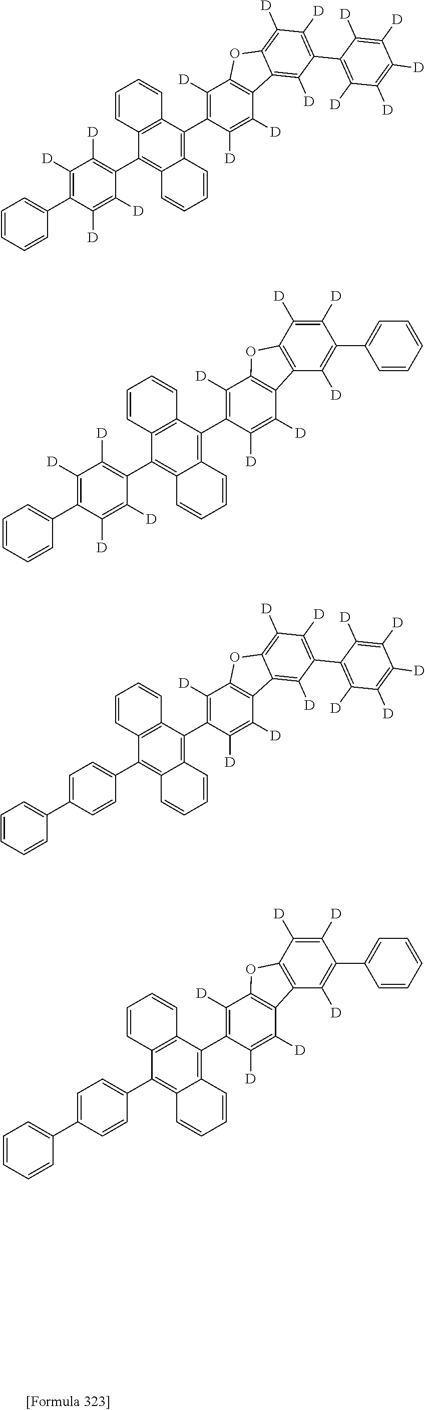

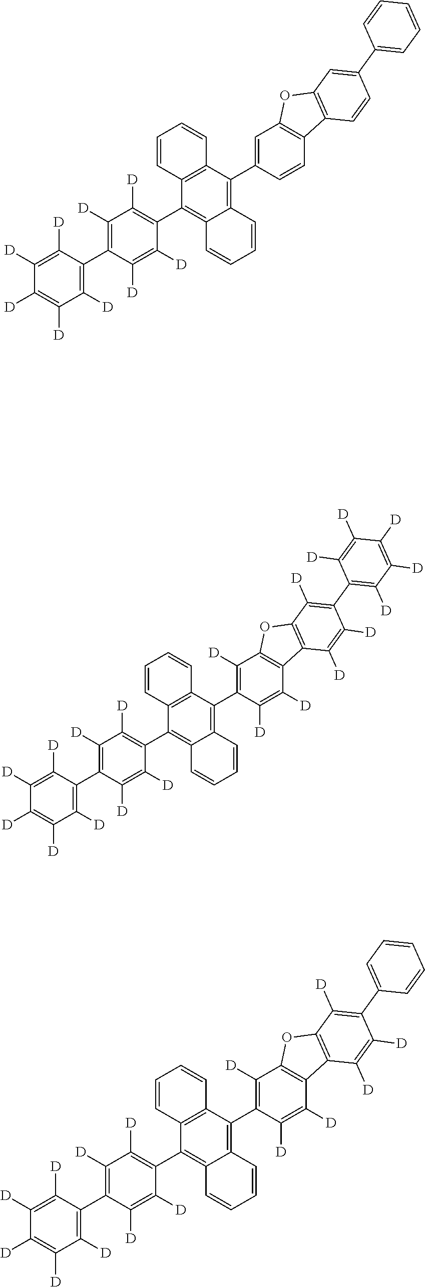 [SMILES: [2H]c1c([2H])c([2H])c(-c2c([2H])c([2H])c(-c3c4ccccc4c(-c4ccc5c(c4)oc4cc(-c6ccccc6)ccc45)c4ccccc34)c([2H])c2[2H])c([2H])c1[2H] ; [2H]c1c([2H])c([2H])c(-c2c([2H])c([2H])c(-c3c4ccccc4c(-c4c([2H])c([2H])c5c(oc6c([2H])c(-c7c([2H])c([2H])c([2H])c([2H])c7[2H])c([2H])c([2H])c65)c4[2H])c4ccccc34)c([2H])c2[2H])c([2H])c1[2H] ; [2H]c1c([2H])c([2H])c(-c2c([2H])c([2H])c(-c3c4ccccc4c(-c4c([2H])c([2H])c5c(oc6c([2H])c(-c7ccccc7)c([2H])c([2H])c65)c4[2H])c4ccccc34)c([2H])c2[2H])c([2H])c1[2H]]

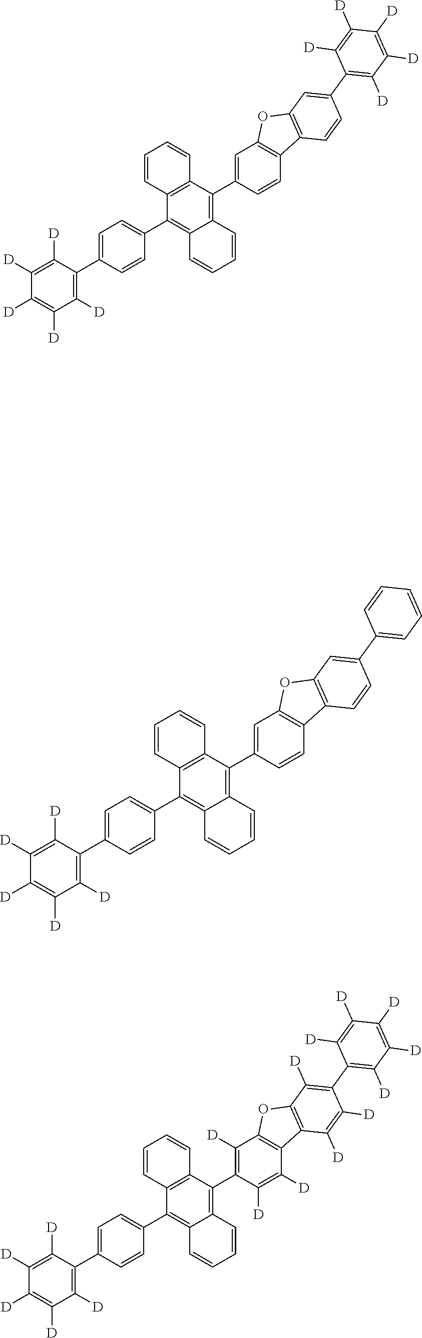 [SMILES: [2H]c1c([2H])c([2H])c(-c2ccc(-c3c4ccccc4c(-c4ccc5c(c4)oc4cc(-c6c([2H])c([2H])c([2H])c([2H])c6[2H])ccc45)c4ccccc34)cc2)c([2H])c1[2H] ; [2H]c1c([2H])c([2H])c(-c2ccc(-c3c4ccccc4c(-c4ccc5c(c4)oc4cc(-c6ccccc6)ccc45)c4ccccc34)cc2)c([2H])c1[2H] ; [2H]c1c([2H])c([2H])c(-c2ccc(-c3c4ccccc4c(-c4c([2H])c([2H])c5c(oc6c([2H])c(-c7c([2H])c([2H])c([2H])c([2H])c7[2H])c([2H])c([2H])c65)c4[2H])c4ccccc34)cc2)c([2H])c1[2H]]

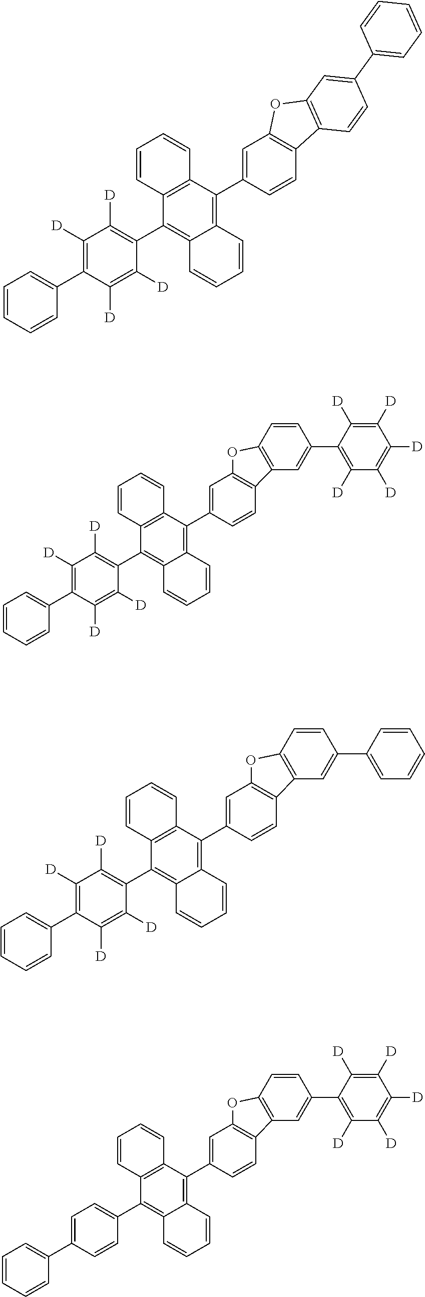 [SMILES: [2H]c1c([2H])c(-c2c3ccccc3c(-c3ccc4c(c3)oc3cc(-c5ccccc5)ccc34)c3ccccc23)c([2H])c([2H])c1-c1ccccc1 ; [2H]c1c([2H])c([2H])c(-c2ccc3oc4cc(-c5c6ccccc6c(-c6c([2H])c([2H])c(-c7ccccc7)c([2H])c6[2H])c6ccccc56)ccc4c3c2)c([2H])c1[2H] ; [2H]c1c([2H])c(-c2c3ccccc3c(-c3ccc4c(c3)oc3ccc(-c5ccccc5)cc34)c3ccccc23)c([2H])c([2H])c1-c1ccccc1 ; [2H]c1c([2H])c([2H])c(-c2ccc3oc4cc(-c5c6ccccc6c(-c6ccc(-c7ccccc7)cc6)c6ccccc56)ccc4c3c2)c([2H])c1[2H]]

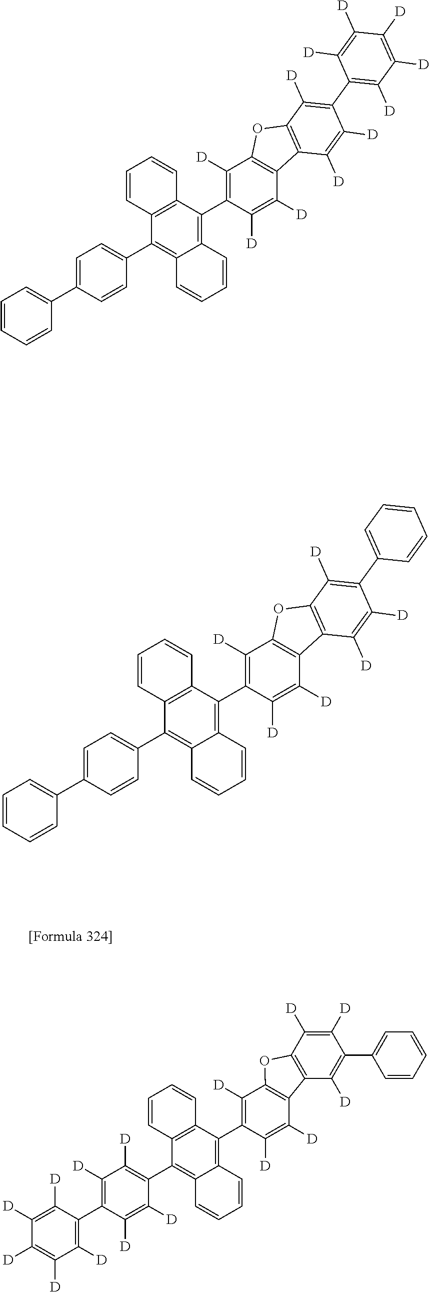 [SMILES: [2H]c1c([2H])c([2H])c(-c2c([2H])c([2H])c3c(oc4c([2H])c(-c5c6ccccc6c(-c6ccc(-c7ccccc7)cc6)c6ccccc56)c([2H])c([2H])c43)c2[2H])c([2H])c1[2H] ; [2H]c1c(-c2ccccc2)c([2H])c2oc3c([2H])c(-c4c5ccccc5c(-c5ccc(-c6ccccc6)cc5)c5ccccc45)c([2H])c([2H])c3c2c1[2H] ; [2H]c1c([2H])c([2H])c(-c2c([2H])c([2H])c(-c3c4ccccc4c(-c4c([2H])c([2H])c5c(oc6c([2H])c([2H])c(-c7ccccc7)c([2H])c65)c4[2H])c4ccccc34)c([2H])c2[2H])c([2H])c1[2H]]

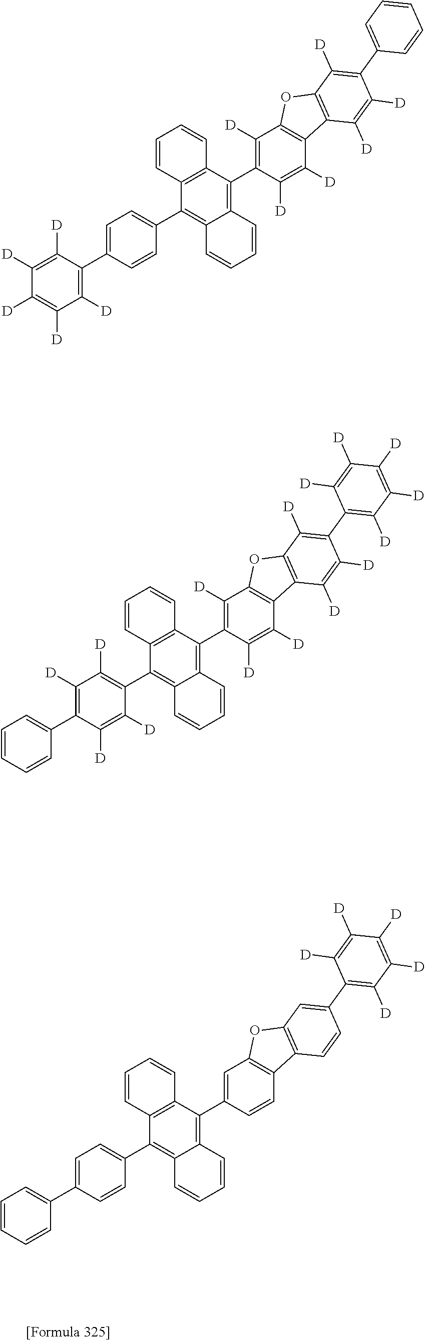 [SMILES: [2H]c1c([2H])c([2H])c(-c2ccc(-c3c4ccccc4c(-c4c([2H])c([2H])c5c(oc6c([2H])c(-c7ccccc7)c([2H])c([2H])c65)c4[2H])c4ccccc34)cc2)c([2H])c1[2H] ; [2H]c1c([2H])c([2H])c(-c2c([2H])c([2H])c3c(oc4c([2H])c(-c5c6ccccc6c(-c6c([2H])c([2H])c(-c7ccccc7)c([2H])c6[2H])c6ccccc56)c([2H])c([2H])c43)c2[2H])c([2H])c1[2H] ; [2H]c1c([2H])c([2H])c(-c2ccc3c(c2)oc2cc(-c4c5ccccc5c(-c5ccc(-c6ccccc6)cc5)c5ccccc45)ccc23)c([2H])c1[2H]]

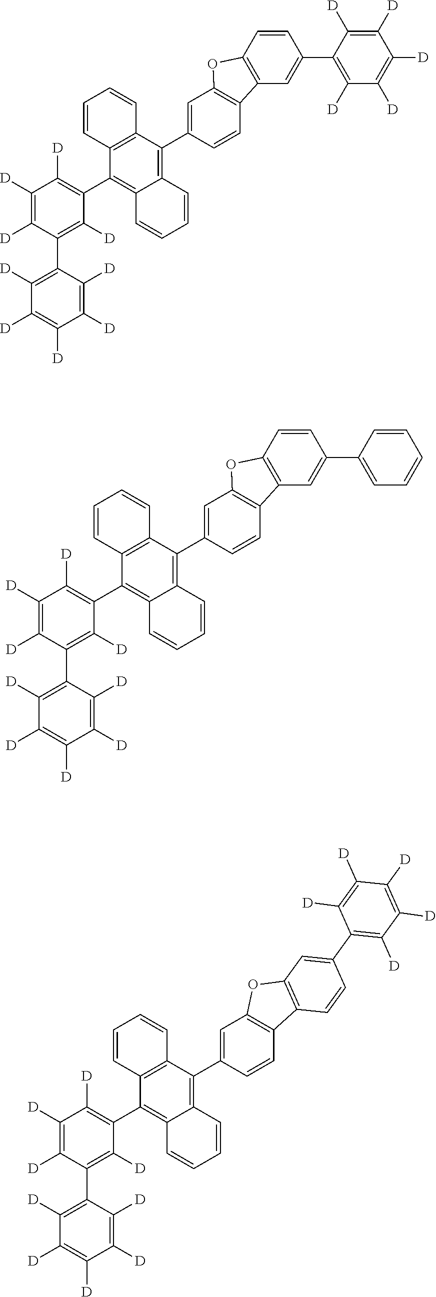 [SMILES: [2H]c1c([2H])c([2H])c(-c2ccc3oc4cc(-c5c6ccccc6c(-c6c([2H])c([2H])c([2H])c(-c7c([2H])c([2H])c([2H])c([2H])c7[2H])c6[2H])c6ccccc56)ccc4c3c2)c([2H])c1[2H] ; [2H]c1c([2H])c([2H])c(-c2c([2H])c([2H])c([2H])c(-c3c4ccccc4c(-c4ccc5c(c4)oc4ccc(-c6ccccc6)cc45)c4ccccc34)c2[2H])c([2H])c1[2H] ; [2H]c1c([2H])c([2H])c(-c2ccc3c(c2)oc2cc(-c4c5ccccc5c(-c5c([2H])c([2H])c([2H])c(-c6c([2H])c([2H])c([2H])c([2H])c6[2H])c5[2H])c5ccccc45)ccc23)c([2H])c1[2H]]

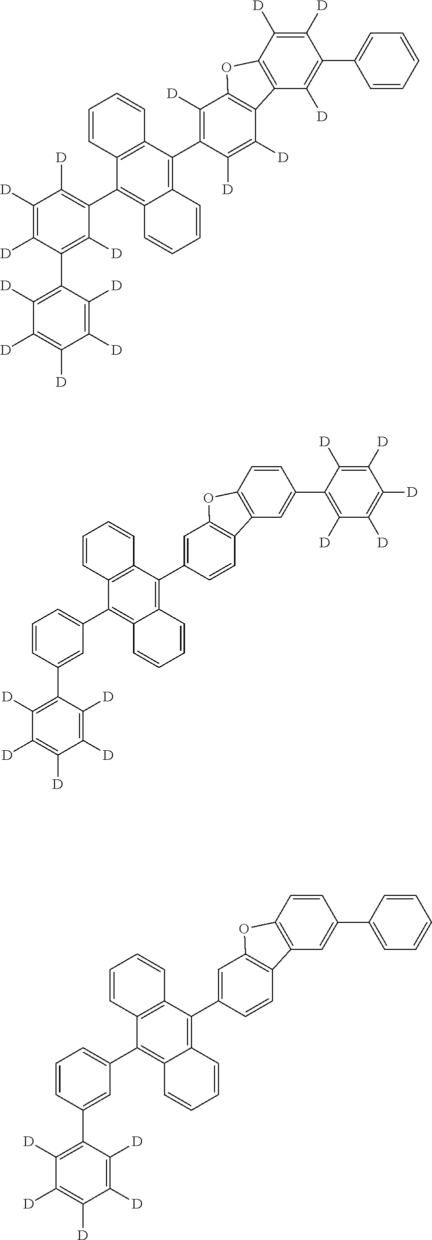 [SMILES: [2H]c1c([2H])c([2H])c(-c2c([2H])c([2H])c([2H])c(-c3c4ccccc4c(-c4c([2H])c([2H])c5c(oc6c([2H])c([2H])c(-c7ccccc7)c([2H])c65)c4[2H])c4ccccc34)c2[2H])c([2H])c1[2H] ; [2H]c1c([2H])c([2H])c(-c2cccc(-c3c4ccccc4c(-c4ccc5c(c4)oc4ccc(-c6c([2H])c([2H])c([2H])c([2H])c6[2H])cc45)c4ccccc34)c2)c([2H])c1[2H] ; [2H]c1c([2H])c([2H])c(-c2cccc(-c3c4ccccc4c(-c4ccc5c(c4)oc4ccc(-c6ccccc6)cc45)c4ccccc34)c2)c([2H])c1[2H]]

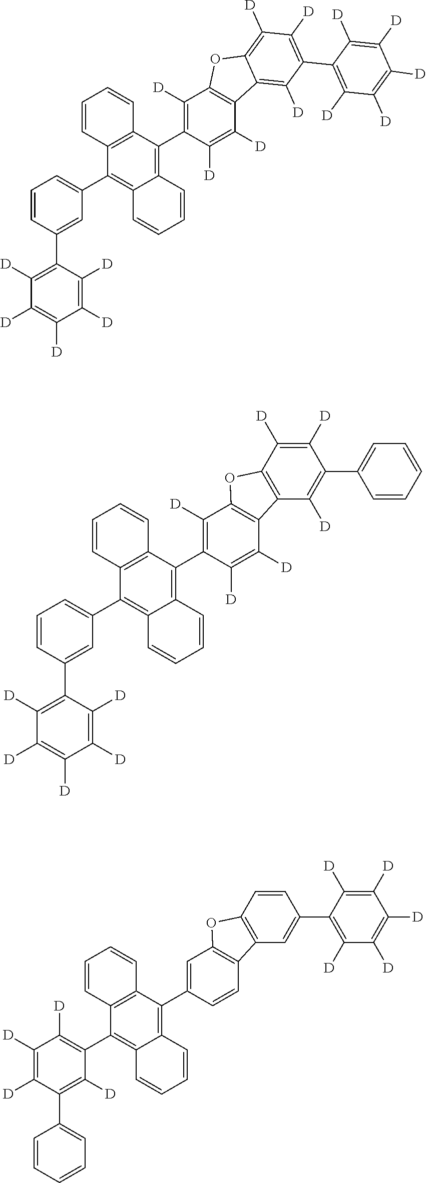 [SMILES: [2H]c1c([2H])c([2H])c(-c2cccc(-c3c4ccccc4c(-c4c([2H])c([2H])c5c(oc6c([2H])c([2H])c(-c7c([2H])c([2H])c([2H])c([2H])c7[2H])c([2H])c65)c4[2H])c4ccccc34)c2)c([2H])c1[2H] ; [2H]c1c([2H])c([2H])c(-c2cccc(-c3c4ccccc4c(-c4c([2H])c([2H])c5c(oc6c([2H])c([2H])c(-c7ccccc7)c([2H])c65)c4[2H])c4ccccc34)c2)c([2H])c1[2H] ; [2H]c1c([2H])c([2H])c(-c2ccc3oc4cc(-c5c6ccccc6c(-c6c([2H])c([2H])c([2H])c(-c7ccccc7)c6[2H])c6ccccc56)ccc4c3c2)c([2H])c1[2H]]

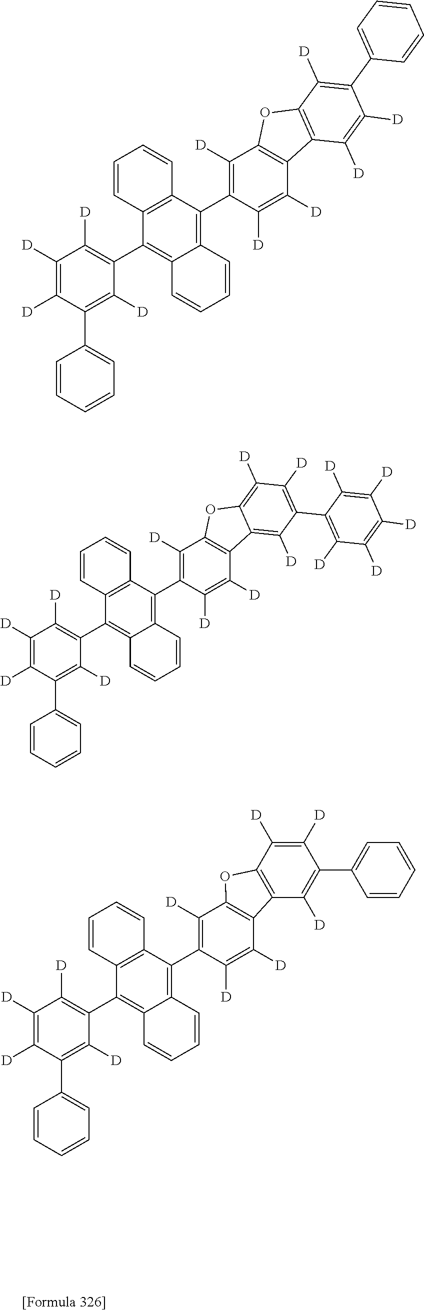 [SMILES: [2H]c1c([2H])c(-c2ccccc2)c([2H])c(-c2c3ccccc3c(-c3c([2H])c([2H])c4c(oc5c([2H])c(-c6ccccc6)c([2H])c([2H])c54)c3[2H])c3ccccc23)c1[2H] ; [2H]c1c([2H])c([2H])c(-c2c([2H])c([2H])c3oc4c([2H])c(-c5c6ccccc6c(-c6c([2H])c([2H])c([2H])c(-c7ccccc7)c6[2H])c6ccccc56)c([2H])c([2H])c4c3c2[2H])c([2H])c1[2H] ; [2H]c1c([2H])c(-c2ccccc2)c([2H])c(-c2c3ccccc3c(-c3c([2H])c([2H])c4c(oc5c([2H])c([2H])c(-c6ccccc6)c([2H])c54)c3[2H])c3ccccc23)c1[2H]]

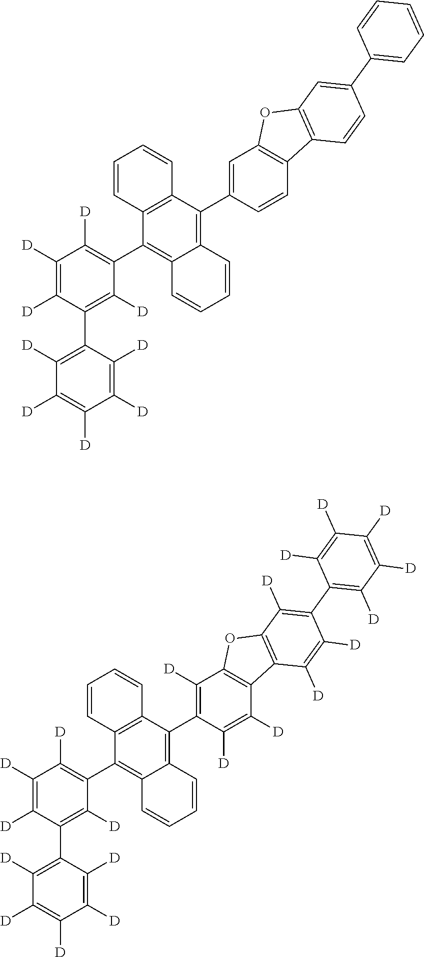 [SMILES: [2H]c1c([2H])c([2H])c(-c2c([2H])c([2H])c([2H])c(-c3c4ccccc4c(-c4ccc5c(c4)oc4cc(-c6ccccc6)ccc45)c4ccccc34)c2[2H])c([2H])c1[2H] ; [2H]c1c([2H])c([2H])c(-c2c([2H])c([2H])c([2H])c(-c3c4ccccc4c(-c4c([2H])c([2H])c5c(oc6c([2H])c(-c7c([2H])c([2H])c([2H])c([2H])c7[2H])c([2H])c([2H])c65)c4[2H])c4ccccc34)c2[2H])c([2H])c1[2H]]

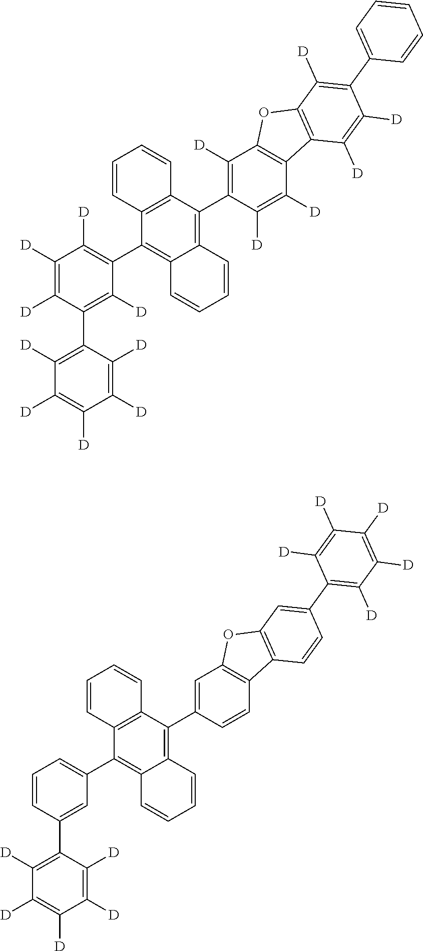 [SMILES: [2H]c1c([2H])c([2H])c(-c2c([2H])c([2H])c([2H])c(-c3c4ccccc4c(-c4c([2H])c([2H])c5c(oc6c([2H])c(-c7ccccc7)c([2H])c([2H])c65)c4[2H])c4ccccc34)c2[2H])c([2H])c1[2H] ; [2H]c1c([2H])c([2H])c(-c2cccc(-c3c4ccccc4c(-c4ccc5c(c4)oc4cc(-c6c([2H])c([2H])c([2H])c([2H])c6[2H])ccc45)c4ccccc34)c2)c([2H])c1[2H]]

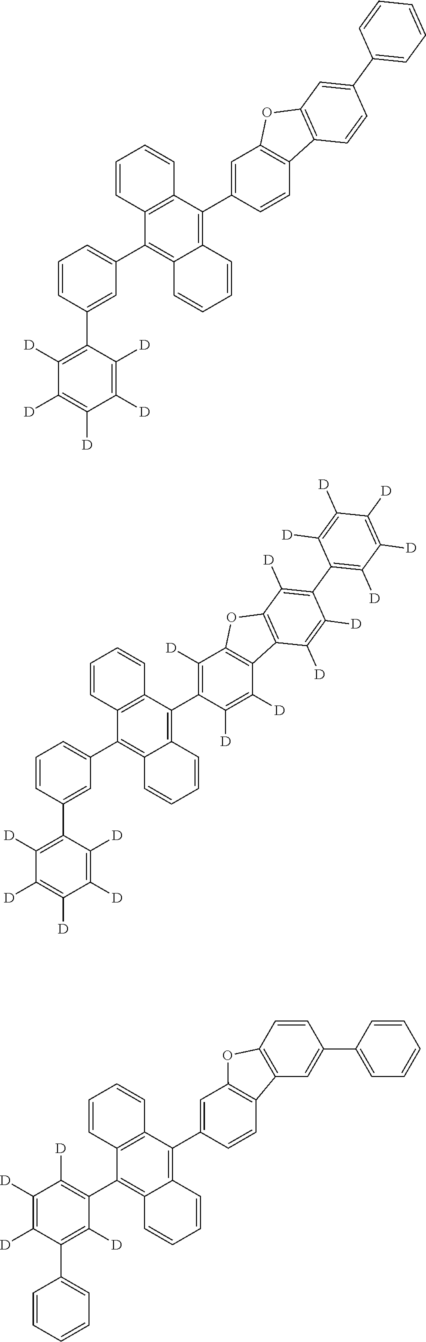 [SMILES: [2H]c1c([2H])c([2H])c(-c2cccc(-c3c4ccccc4c(-c4ccc5c(c4)oc4cc(-c6ccccc6)ccc45)c4ccccc34)c2)c([2H])c1[2H] ; [2H]c1c([2H])c([2H])c(-c2cccc(-c3c4ccccc4c(-c4c([2H])c([2H])c5c(oc6c([2H])c(-c7c([2H])c([2H])c([2H])c([2H])c7[2H])c([2H])c([2H])c65)c4[2H])c4ccccc34)c2)c([2H])c1[2H] ; [2H]c1c([2H])c(-c2ccccc2)c([2H])c(-c2c3ccccc3c(-c3ccc4c(c3)oc3ccc(-c5ccccc5)cc34)c3ccccc23)c1[2H]]

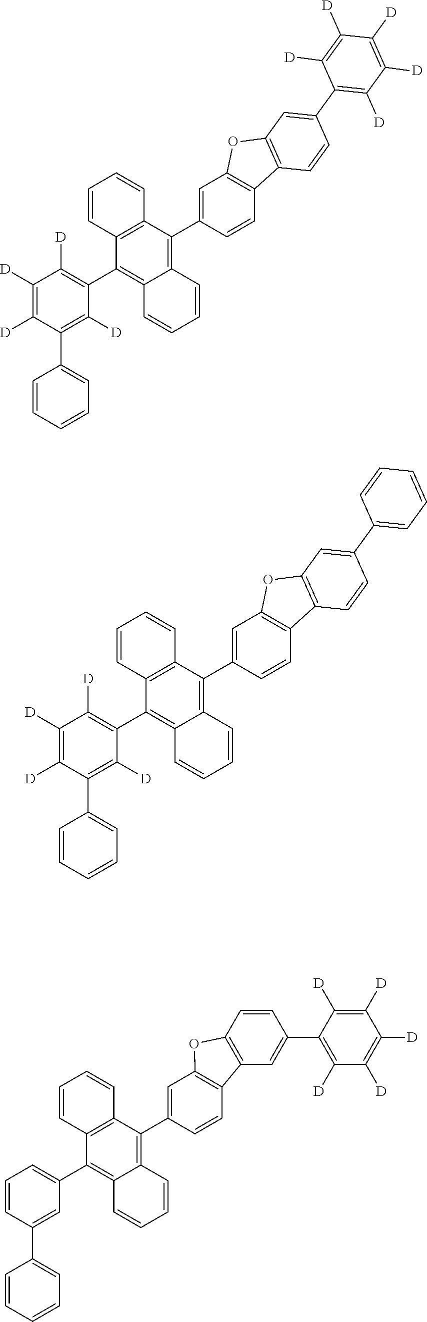 [SMILES: [2H]c1c([2H])c([2H])c(-c2ccc3c(c2)oc2cc(-c4c5ccccc5c(-c5c([2H])c([2H])c([2H])c(-c6ccccc6)c5[2H])c5ccccc45)ccc23)c([2H])c1[2H] ; [2H]c1c([2H])c(-c2ccccc2)c([2H])c(-c2c3ccccc3c(-c3ccc4c(c3)oc3cc(-c5ccccc5)ccc34)c3ccccc23)c1[2H] ; [2H]c1c([2H])c([2H])c(-c2ccc3oc4cc(-c5c6ccccc6c(-c6cccc(-c7ccccc7)c6)c6ccccc56)ccc4c3c2)c([2H])c1[2H]]

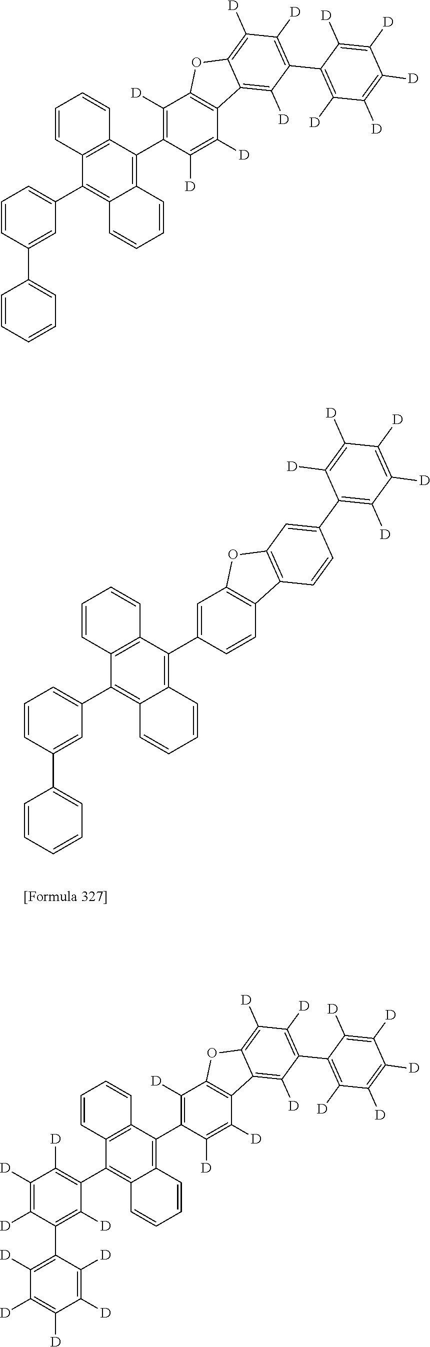 [SMILES: [2H]c1c([2H])c([2H])c(-c2c([2H])c([2H])c3oc4c([2H])c(-c5c6ccccc6c(-c6cccc(-c7ccccc7)c6)c6ccccc56)c([2H])c([2H])c4c3c2[2H])c([2H])c1[2H] ; [2H]c1c([2H])c([2H])c(-c2ccc3c(c2)oc2cc(-c4c5ccccc5c(-c5cccc(-c6ccccc6)c5)c5ccccc45)ccc23)c([2H])c1[2H] ; [2H]c1c([2H])c([2H])c(-c2c([2H])c([2H])c([2H])c(-c3c4ccccc4c(-c4c([2H])c([2H])c5c(oc6c([2H])c([2H])c(-c7c([2H])c([2H])c([2H])c([2H])c7[2H])c([2H])c65)c4[2H])c4ccccc34)c2[2H])c([2H])c1[2H]]

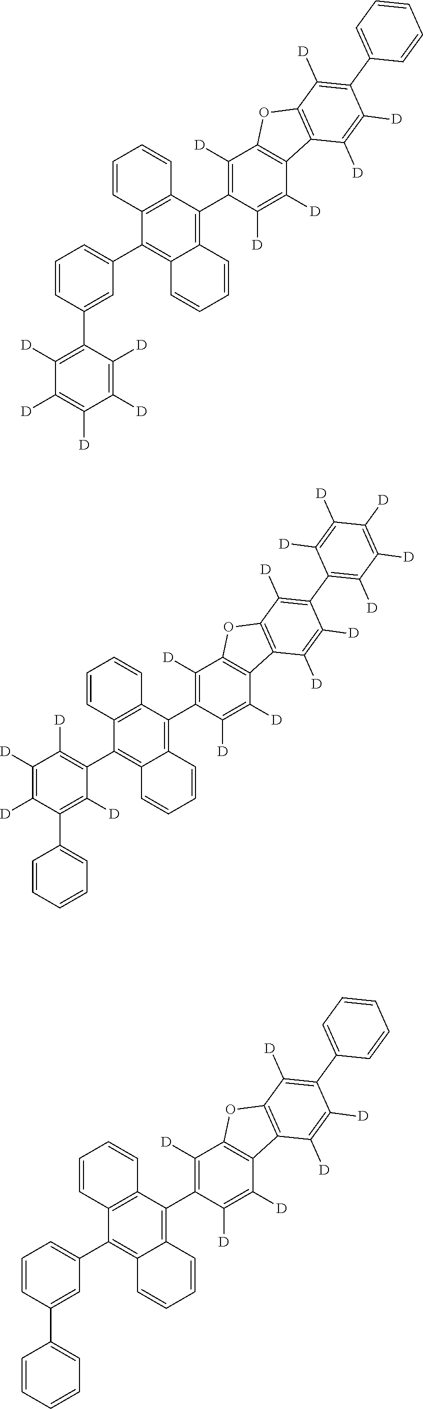 [SMILES: [2H]c1c([2H])c([2H])c(-c2cccc(-c3c4ccccc4c(-c4c([2H])c([2H])c5c(oc6c([2H])c(-c7ccccc7)c([2H])c([2H])c65)c4[2H])c4ccccc34)c2)c([2H])c1[2H] ; [2H]c1c([2H])c([2H])c(-c2c([2H])c([2H])c3c(oc4c([2H])c(-c5c6ccccc6c(-c6c([2H])c([2H])c([2H])c(-c7ccccc7)c6[2H])c6ccccc56)c([2H])c([2H])c43)c2[2H])c([2H])c1[2H] ; [2H]c1c(-c2ccccc2)c([2H])c2oc3c([2H])c(-c4c5ccccc5c(-c5cccc(-c6ccccc6)c5)c5ccccc45)c([2H])c([2H])c3c2c1[2H]]

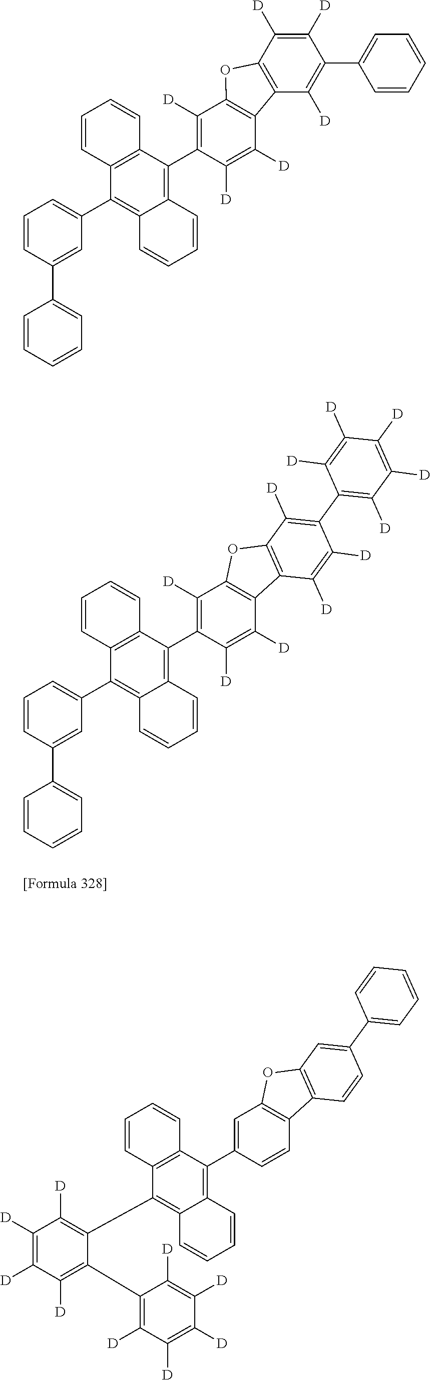 [SMILES: [2H]c1c(-c2ccccc2)c([2H])c2c(oc3c([2H])c(-c4c5ccccc5c(-c5cccc(-c6ccccc6)c5)c5ccccc45)c([2H])c([2H])c32)c1[2H] ; [2H]c1c([2H])c([2H])c(-c2c([2H])c([2H])c3c(oc4c([2H])c(-c5c6ccccc6c(-c6cccc(-c7ccccc7)c6)c6ccccc56)c([2H])c([2H])c43)c2[2H])c([2H])c1[2H] ; [2H]c1c([2H])c([2H])c(-c2c([2H])c([2H])c([2H])c([2H])c2-c2c3ccccc3c(-c3ccc4c(c3)oc3cc(-c5ccccc5)ccc34)c3ccccc23)c([2H])c1[2H]]

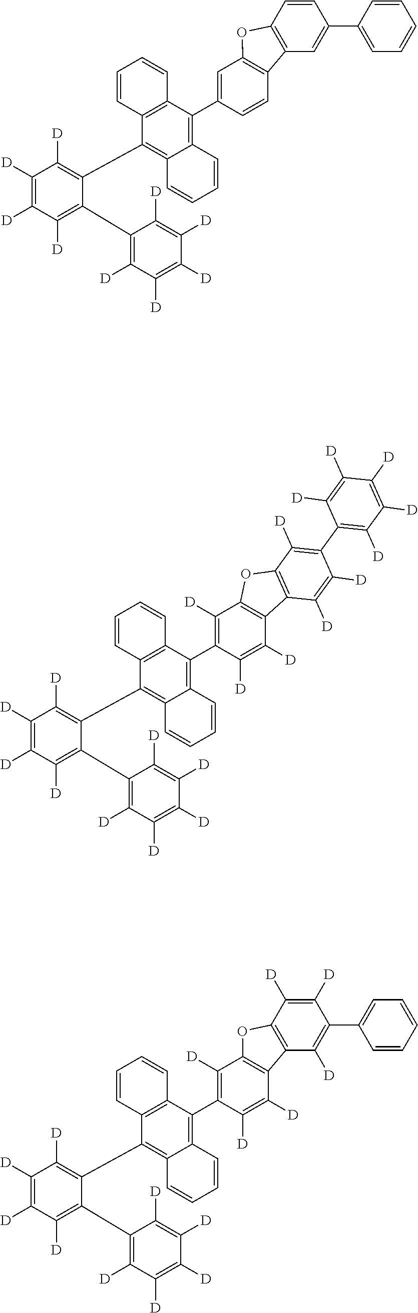 [SMILES: [2H]c1c([2H])c([2H])c(-c2c([2H])c([2H])c([2H])c([2H])c2-c2c3ccccc3c(-c3ccc4c(c3)oc3ccc(-c5ccccc5)cc34)c3ccccc23)c([2H])c1[2H] ; [2H]c1c([2H])c([2H])c(-c2c([2H])c([2H])c([2H])c([2H])c2-c2c3ccccc3c(-c3c([2H])c([2H])c4c(oc5c([2H])c(-c6c([2H])c([2H])c([2H])c([2H])c6[2H])c([2H])c([2H])c54)c3[2H])c3ccccc23)c([2H])c1[2H] ; [2H]c1c([2H])c([2H])c(-c2c([2H])c([2H])c([2H])c([2H])c2-c2c3ccccc3c(-c3c([2H])c([2H])c4c(oc5c([2H])c([2H])c(-c6ccccc6)c([2H])c54)c3[2H])c3ccccc23)c([2H])c1[2H]]

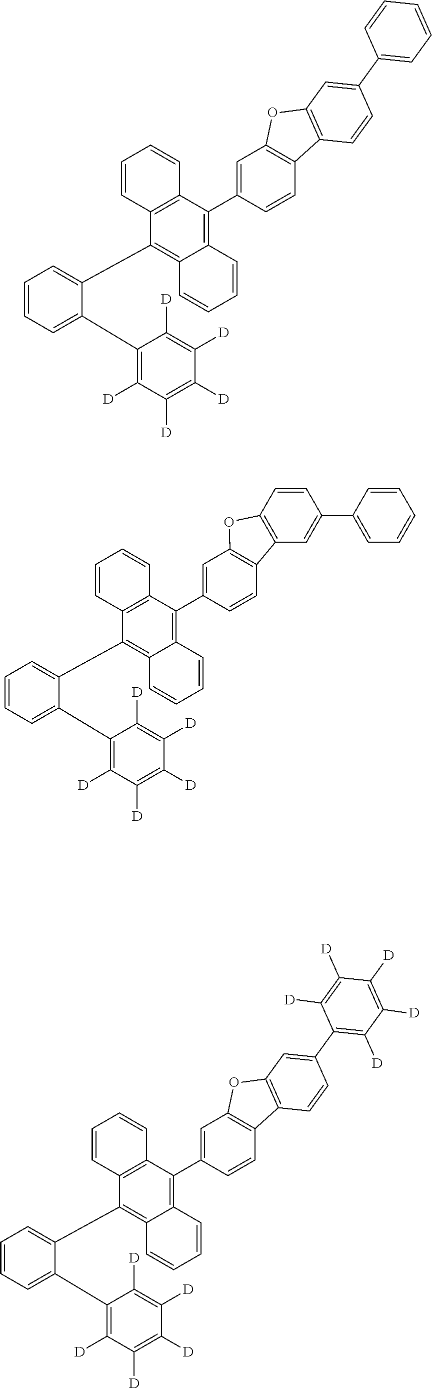 [SMILES: [2H]c1c([2H])c([2H])c(-c2ccccc2-c2c3ccccc3c(-c3ccc4c(c3)oc3cc(-c5ccccc5)ccc34)c3ccccc23)c([2H])c1[2H] ; [2H]c1c([2H])c([2H])c(-c2ccccc2-c2c3ccccc3c(-c3ccc4c(c3)oc3ccc(-c5ccccc5)cc34)c3ccccc23)c([2H])c1[2H] ; [2H]c1c([2H])c([2H])c(-c2ccc3c(c2)oc2cc(-c4c5ccccc5c(-c5ccccc5-c5c([2H])c([2H])c([2H])c([2H])c5[2H])c5ccccc45)ccc23)c([2H])c1[2H]]

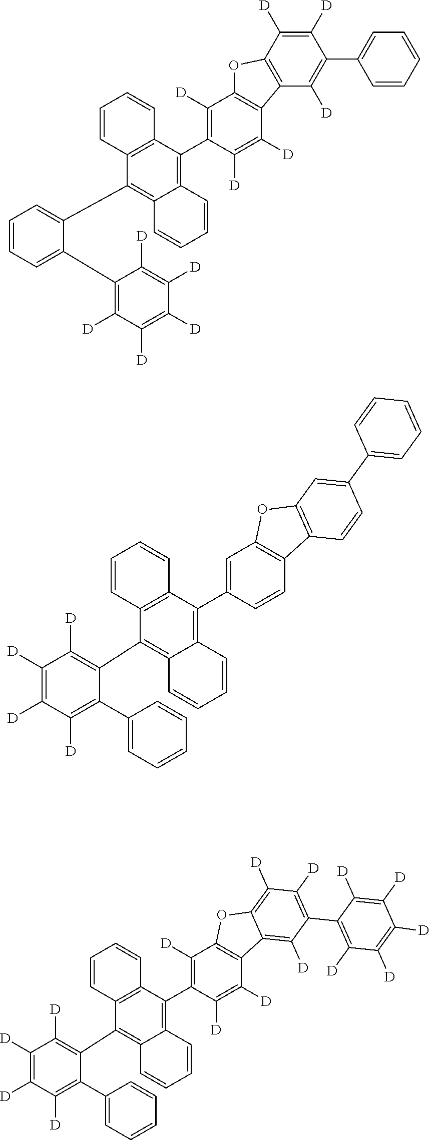 [SMILES: [2H]c1c([2H])c([2H])c(-c2ccccc2-c2c3ccccc3c(-c3c([2H])c([2H])c4c(oc5c([2H])c([2H])c(-c6ccccc6)c([2H])c54)c3[2H])c3ccccc23)c([2H])c1[2H] ; [2H]c1c([2H])c([2H])c(-c2c3ccccc3c(-c3ccc4c(c3)oc3cc(-c5ccccc5)ccc34)c3ccccc23)c(-c2ccccc2)c1[2H] ; [2H]c1c([2H])c([2H])c(-c2c([2H])c([2H])c3oc4c([2H])c(-c5c6ccccc6c(-c6c([2H])c([2H])c([2H])c([2H])c6-c6ccccc6)c6ccccc56)c([2H])c([2H])c4c3c2[2H])c([2H])c1[2H]]

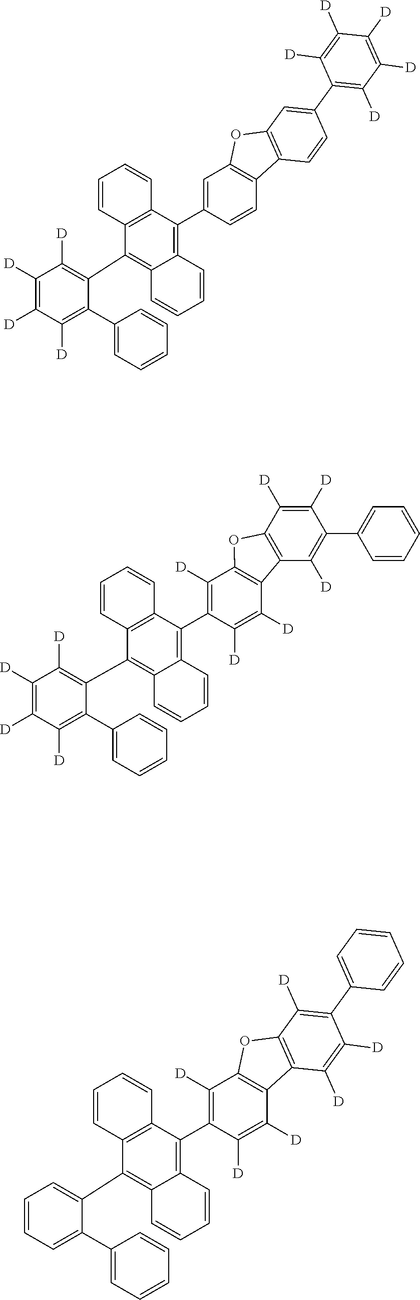 [SMILES: [2H]c1c([2H])c([2H])c(-c2ccc3c(c2)oc2cc(-c4c5ccccc5c(-c5c([2H])c([2H])c([2H])c([2H])c5-c5ccccc5)c5ccccc45)ccc23)c([2H])c1[2H] ; [2H]c1c([2H])c([2H])c(-c2c3ccccc3c(-c3c([2H])c([2H])c4c(oc5c([2H])c([2H])c(-c6ccccc6)c([2H])c54)c3[2H])c3ccccc23)c(-c2ccccc2)c1[2H] ; [2H]c1c(-c2ccccc2)c([2H])c2oc3c([2H])c(-c4c5ccccc5c(-c5ccccc5-c5ccccc5)c5ccccc45)c([2H])c([2H])c3c2c1[2H]]

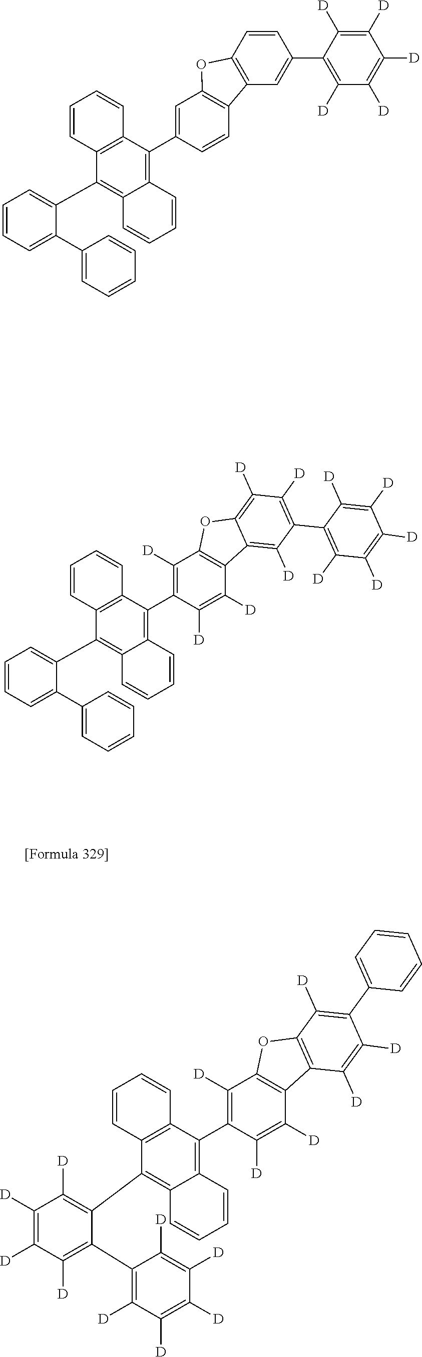 [SMILES: [2H]c1c([2H])c([2H])c(-c2ccc3oc4cc(-c5c6ccccc6c(-c6ccccc6-c6ccccc6)c6ccccc56)ccc4c3c2)c([2H])c1[2H] ; [2H]c1c([2H])c([2H])c(-c2c([2H])c([2H])c3oc4c([2H])c(-c5c6ccccc6c(-c6ccccc6-c6ccccc6)c6ccccc56)c([2H])c([2H])c4c3c2[2H])c([2H])c1[2H] ; [2H]c1c([2H])c([2H])c(-c2c([2H])c([2H])c([2H])c([2H])c2-c2c3ccccc3c(-c3c([2H])c([2H])c4c(oc5c([2H])c(-c6ccccc6)c([2H])c([2H])c54)c3[2H])c3ccccc23)c([2H])c1[2H]]

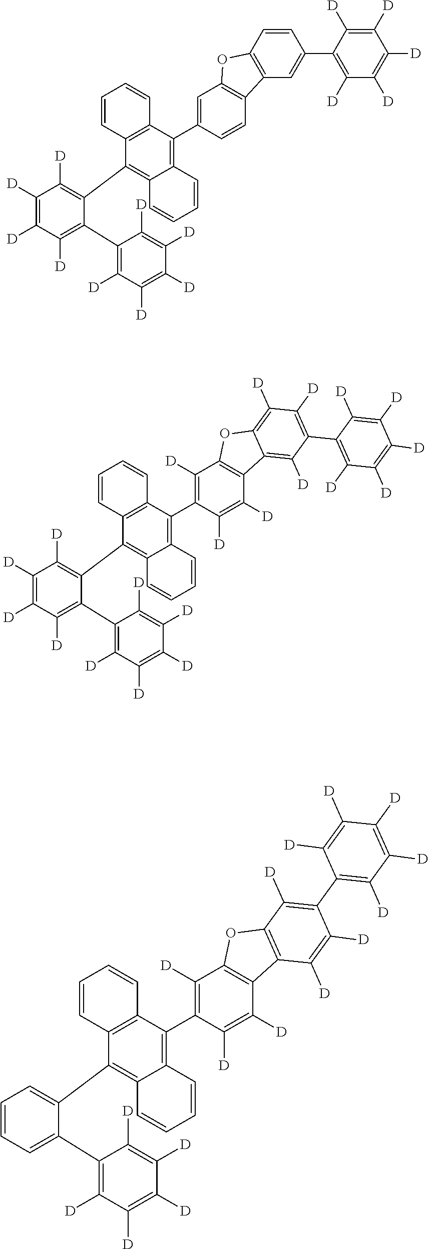

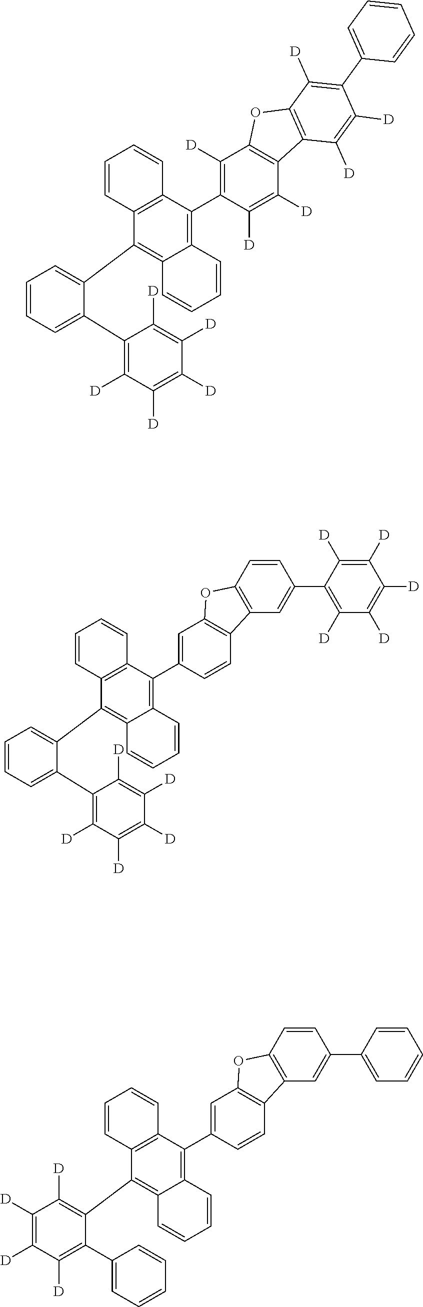 [SMILES: [2H]c1c([2H])c([2H])c(-c2ccccc2-c2c3ccccc3c(-c3c([2H])c([2H])c4c(oc5c([2H])c(-c6ccccc6)c([2H])c([2H])c54)c3[2H])c3ccccc23)c([2H])c1[2H] ; [2H]c1c([2H])c([2H])c(-c2ccc3oc4cc(-c5c6ccccc6c(-c6ccccc6-c6c([2H])c([2H])c([2H])c([2H])c6[2H])c6ccccc56)ccc4c3c2)c([2H])c1[2H] ; [2H]c1c([2H])c([2H])c(-c2c3ccccc3c(-c3ccc4c(c3)oc3ccc(-c5ccccc5)cc34)c3ccccc23)c(-c2ccccc2)c1[2H]]

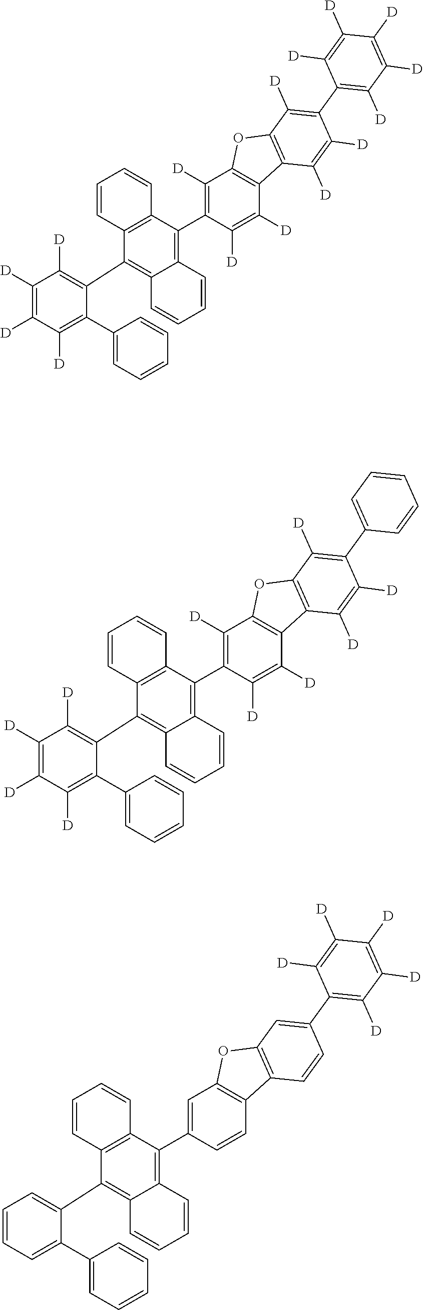 [SMILES: [2H]c1c([2H])c([2H])c(-c2c([2H])c([2H])c3c(oc4c([2H])c(-c5c6ccccc6c(-c6c([2H])c([2H])c([2H])c([2H])c6-c6ccccc6)c6ccccc56)c([2H])c([2H])c43)c2[2H])c([2H])c1[2H] ; [2H]c1c([2H])c([2H])c(-c2c3ccccc3c(-c3c([2H])c([2H])c4c(oc5c([2H])c(-c6ccccc6)c([2H])c([2H])c54)c3[2H])c3ccccc23)c(-c2ccccc2)c1[2H] ; [2H]c1c([2H])c([2H])c(-c2ccc3c(c2)oc2cc(-c4c5ccccc5c(-c5ccccc5-c5ccccc5)c5ccccc45)ccc23)c([2H])c1[2H]]

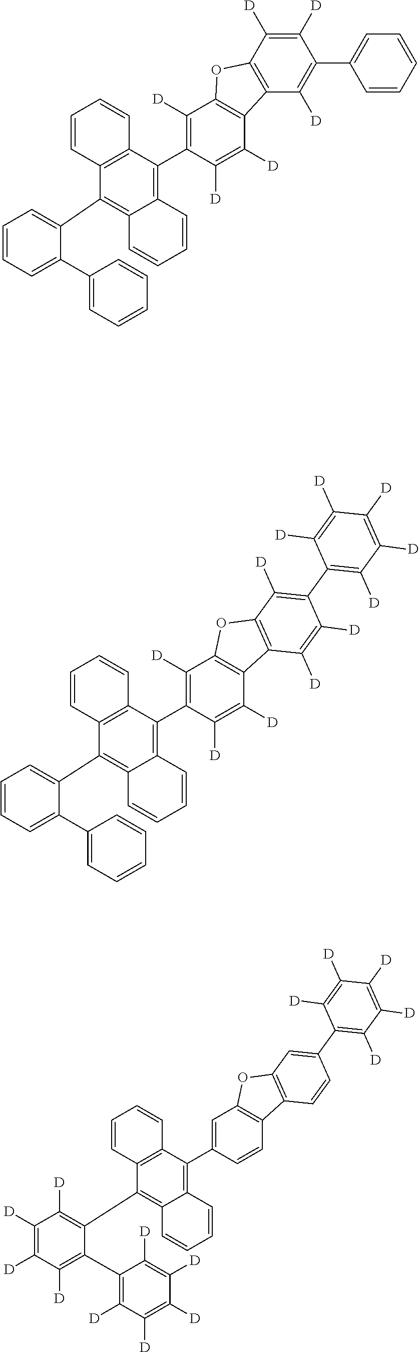 [SMILES: [2H]c1c(-c2ccccc2)c([2H])c2c(oc3c([2H])c(-c4c5ccccc5c(-c5ccccc5-c5ccccc5)c5ccccc45)c([2H])c([2H])c32)c1[2H] ; [2H]c1c([2H])c([2H])c(-c2c([2H])c([2H])c3c(oc4c([2H])c(-c5c6ccccc6c(-c6ccccc6-c6ccccc6)c6ccccc56)c([2H])c([2H])c43)c2[2H])c([2H])c1[2H] ; [2H]c1c([2H])c([2H])c(-c2ccc3c(c2)oc2cc(-c4c5ccccc5c(-c5c([2H])c([2H])c([2H])c([2H])c5-c5c([2H])c([2H])c([2H])c([2H])c5[2H])c5ccccc45)ccc23)c([2H])c1[2H]]

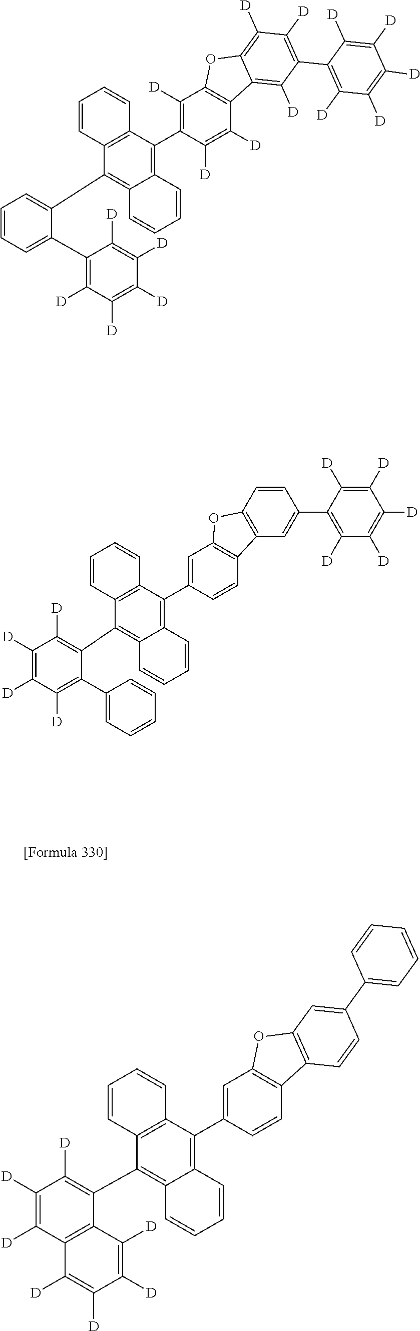 [SMILES: [2H]c1c([2H])c([2H])c(-c2ccccc2-c2c3ccccc3c(-c3c([2H])c([2H])c4c(oc5c([2H])c([2H])c(-c6c([2H])c([2H])c([2H])c([2H])c6[2H])c([2H])c54)c3[2H])c3ccccc23)c([2H])c1[2H] ; [2H]c1c([2H])c([2H])c(-c2ccc3oc4cc(-c5c6ccccc6c(-c6c([2H])c([2H])c([2H])c([2H])c6-c6ccccc6)c6ccccc56)ccc4c3c2)c([2H])c1[2H] ; [2H]c1c([2H])c([2H])c2c(-c3c4ccccc4c(-c4ccc5c(c4)oc4cc(-c6ccccc6)ccc45)c4ccccc34)c([2H])c([2H])c([2H])c2c1[2H]]

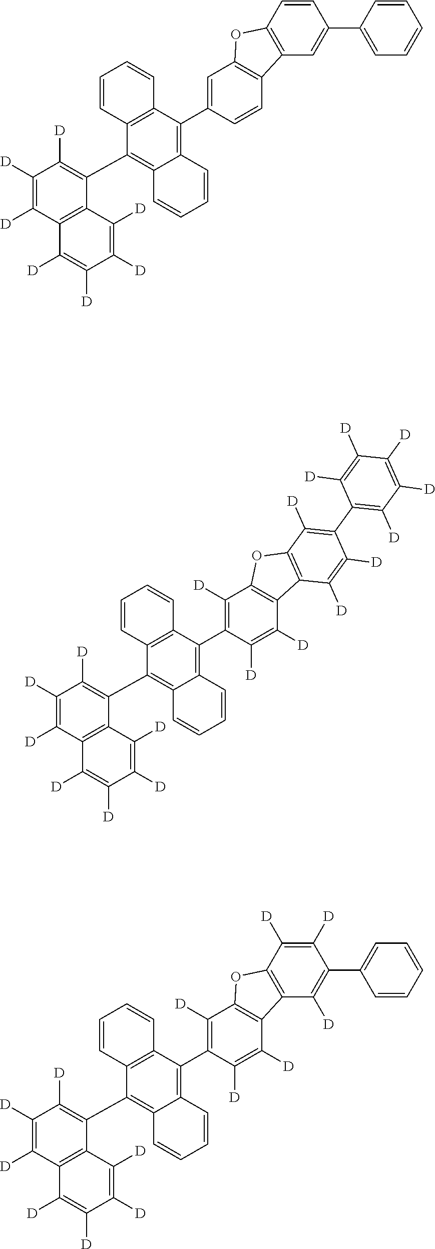 [SMILES: [2H]c1c([2H])c([2H])c2c(-c3c4ccccc4c(-c4ccc5c(c4)oc4ccc(-c6ccccc6)cc45)c4ccccc34)c([2H])c([2H])c([2H])c2c1[2H] ; [2H]c1c([2H])c([2H])c(-c2c([2H])c([2H])c3c(oc4c([2H])c(-c5c6ccccc6c(-c6c([2H])c([2H])c([2H])c7c([2H])c([2H])c([2H])c([2H])c67)c6ccccc56)c([2H])c([2H])c43)c2[2H])c([2H])c1[2H] ; [2H]c1c(-c2ccccc2)c([2H])c2c(oc3c([2H])c(-c4c5ccccc5c(-c5c([2H])c([2H])c([2H])c6c([2H])c([2H])c([2H])c([2H])c56)c5ccccc45)c([2H])c([2H])c32)c1[2H]]

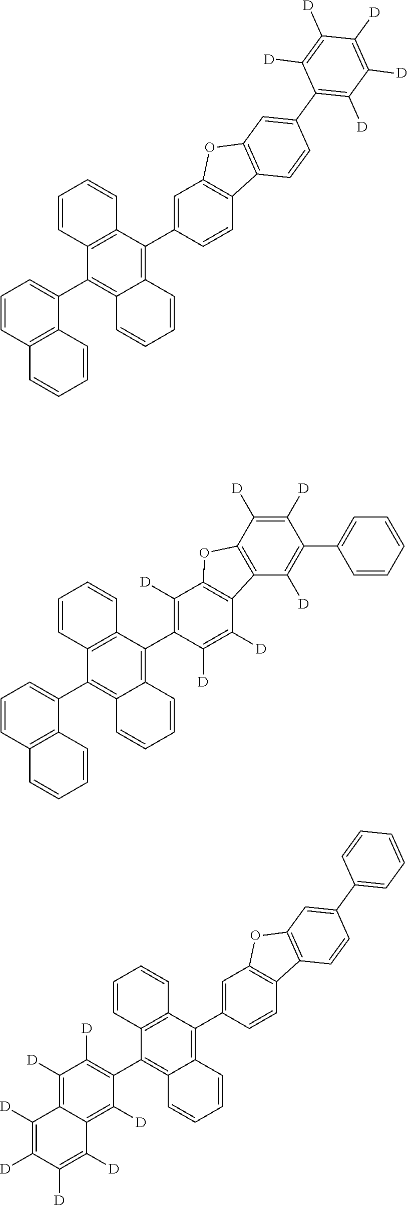 [SMILES: [2H]c1c([2H])c([2H])c(-c2ccc3c(c2)oc2cc(-c4c5ccccc5c(-c5cccc6ccccc56)c5ccccc45)ccc23)c([2H])c1[2H] ; [2H]c1c(-c2ccccc2)c([2H])c2c(oc3c([2H])c(-c4c5ccccc5c(-c5cccc6ccccc56)c5ccccc45)c([2H])c([2H])c32)c1[2H] ; [2H]c1c([2H])c([2H])c2c([2H])c(-c3c4ccccc4c(-c4ccc5c(c4)oc4cc(-c6ccccc6)ccc45)c4ccccc34)c([2H])c([2H])c2c1[2H]]

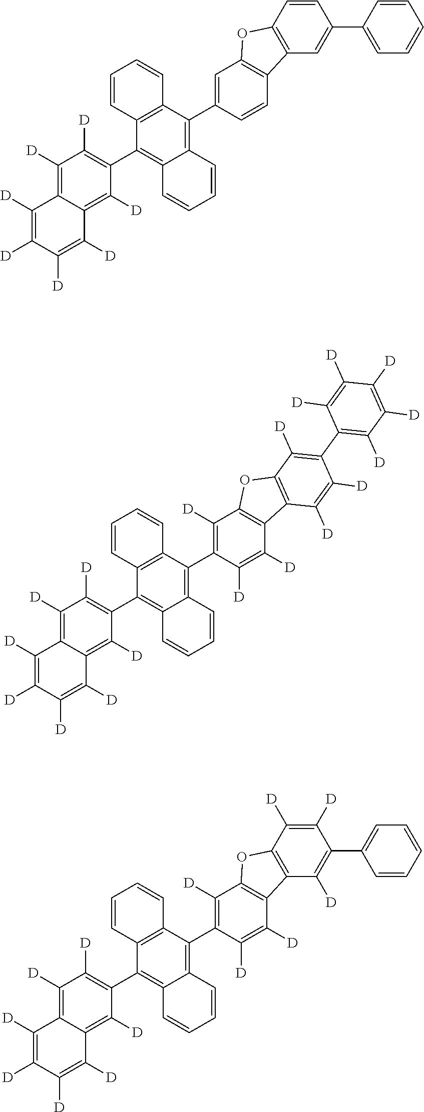 [SMILES: [2H]c1c([2H])c([2H])c2c([2H])c(-c3c4ccccc4c(-c4ccc5c(c4)oc4ccc(-c6ccccc6)cc45)c4ccccc34)c([2H])c([2H])c2c1[2H] ; [2H]c1c([2H])c([2H])c(-c2c([2H])c([2H])c3c(oc4c([2H])c(-c5c6ccccc6c(-c6c([2H])c([2H])c7c([2H])c([2H])c([2H])c([2H])c7c6[2H])c6ccccc56)c([2H])c([2H])c43)c2[2H])c([2H])c1[2H] ; [2H]c1c(-c2ccccc2)c([2H])c2c(oc3c([2H])c(-c4c5ccccc5c(-c5c([2H])c([2H])c6c([2H])c([2H])c([2H])c([2H])c6c5[2H])c5ccccc45)c([2H])c([2H])c32)c1[2H]]

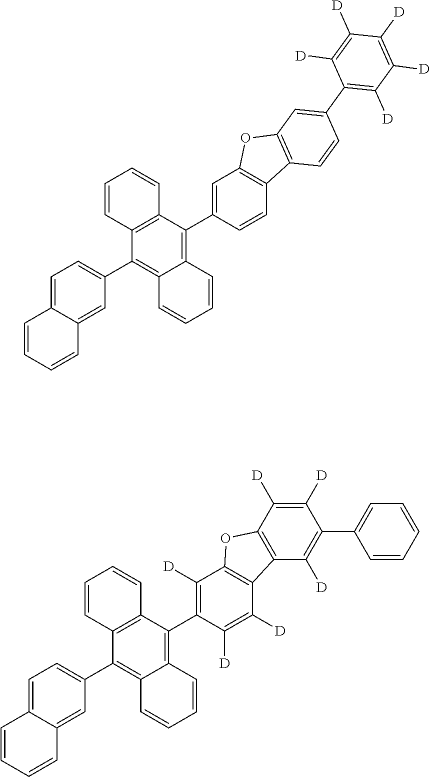 [SMILES: [2H]c1c([2H])c([2H])c(-c2ccc3c(c2)oc2cc(-c4c5ccccc5c(-c5ccc6ccccc6c5)c5ccccc45)ccc23)c([2H])c1[2H] ; [2H]c1c(-c2ccccc2)c([2H])c2c(oc3c([2H])c(-c4c5ccccc5c(-c5ccc6ccccc6c5)c5ccccc45)c([2H])c([2H])c32)c1[2H]]

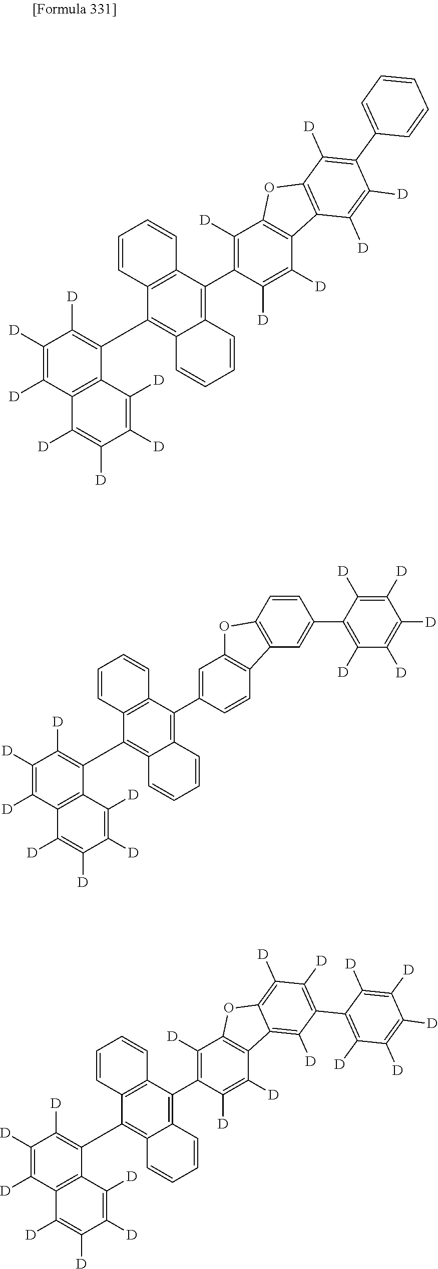 [SMILES: [2H]c1c([2H])c([2H])c2c(-c3c4ccccc4c(-c4c([2H])c([2H])c5c(oc6c([2H])c(-c7ccccc7)c([2H])c([2H])c65)c4[2H])c4ccccc34)c([2H])c([2H])c([2H])c2c1[2H] ; [2H]c1c([2H])c([2H])c(-c2ccc3oc4cc(-c5c6ccccc6c(-c6c([2H])c([2H])c([2H])c7c([2H])c([2H])c([2H])c([2H])c67)c6ccccc56)ccc4c3c2)c([2H])c1[2H] ; [2H]c1c([2H])c([2H])c(-c2c([2H])c([2H])c3oc4c([2H])c(-c5c6ccccc6c(-c6c([2H])c([2H])c([2H])c7c([2H])c([2H])c([2H])c([2H])c67)c6ccccc56)c([2H])c([2H])c4c3c2[2H])c([2H])c1[2H]]

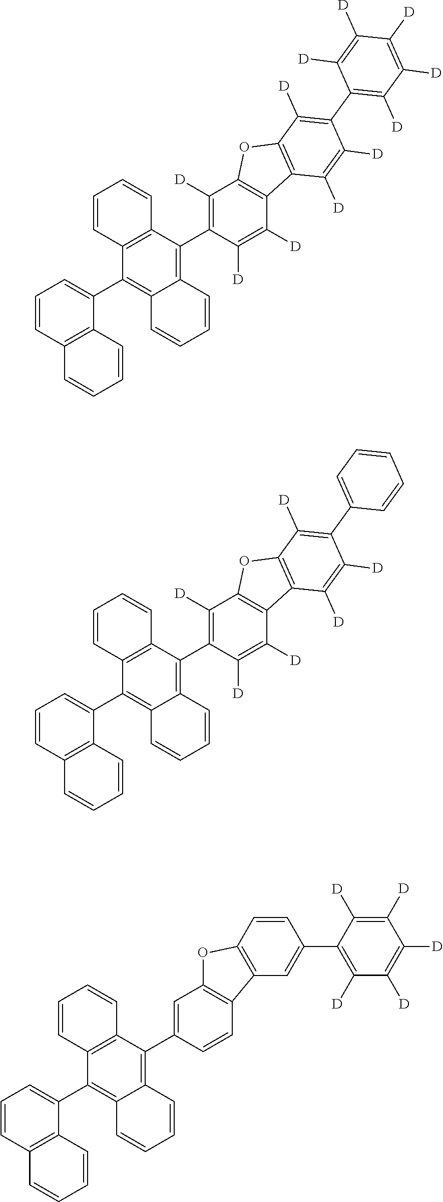 [SMILES: [2H]c1c([2H])c([2H])c(-c2c([2H])c([2H])c3c(oc4c([2H])c(-c5c6ccccc6c(-c6cccc7ccccc67)c6ccccc56)c([2H])c([2H])c43)c2[2H])c([2H])c1[2H] ; [2H]c1c(-c2ccccc2)c([2H])c2oc3c([2H])c(-c4c5ccccc5c(-c5cccc6ccccc56)c5ccccc45)c([2H])c([2H])c3c2c1[2H] ; [2H]c1c([2H])c([2H])c(-c2ccc3oc4cc(-c5c6ccccc6c(-c6cccc7ccccc67)c6ccccc56)ccc4c3c2)c([2H])c1[2H]]

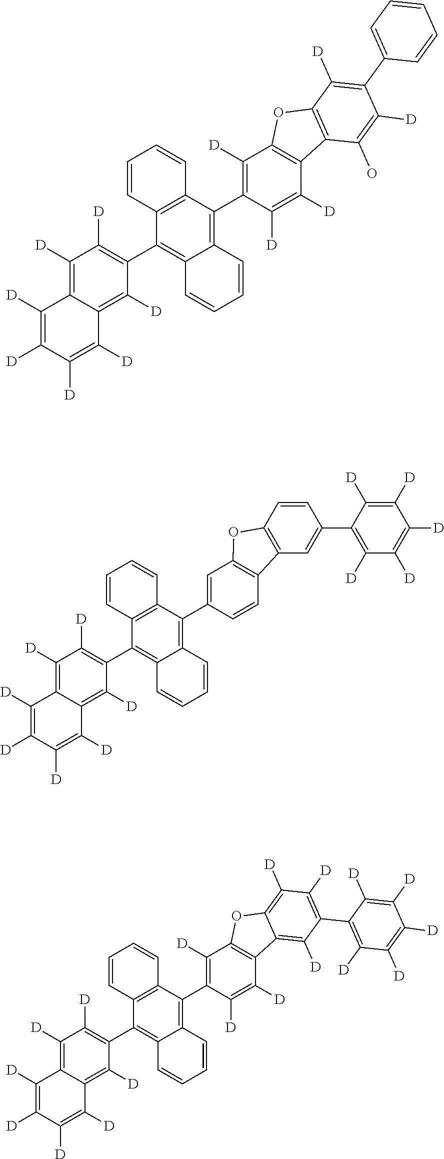

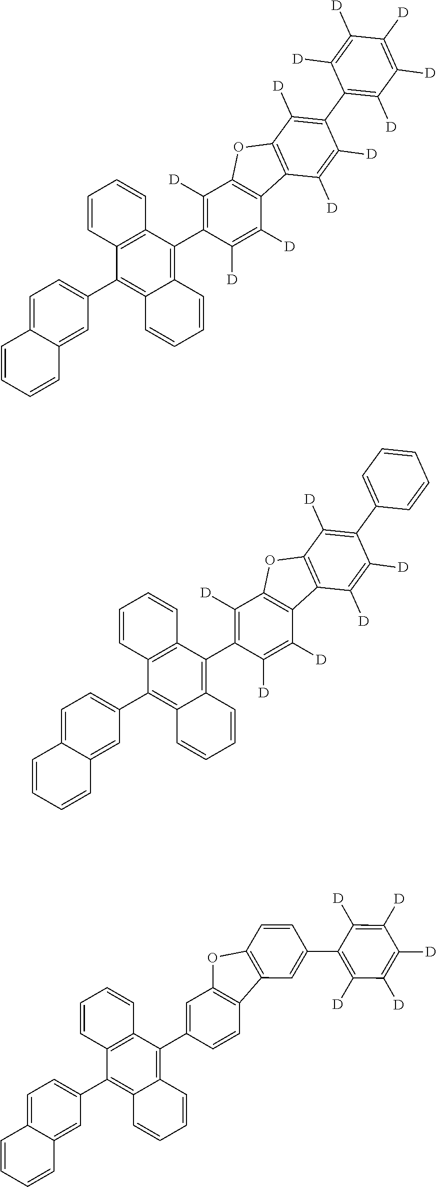 [SMILES: [2H]c1c([2H])c([2H])c(-c2c([2H])c([2H])c3c(oc4c([2H])c(-c5c6ccccc6c(-c6ccc7ccccc7c6)c6ccccc56)c([2H])c([2H])c43)c2[2H])c([2H])c1[2H] ; [2H]c1c(-c2ccccc2)c([2H])c2oc3c([2H])c(-c4c5ccccc5c(-c5ccc6ccccc6c5)c5ccccc45)c([2H])c([2H])c3c2c1[2H] ; [2H]c1c([2H])c([2H])c(-c2ccc3oc4cc(-c5c6ccccc6c(-c6ccc7ccccc7c6)c6ccccc56)ccc4c3c2)c([2H])c1[2H]]

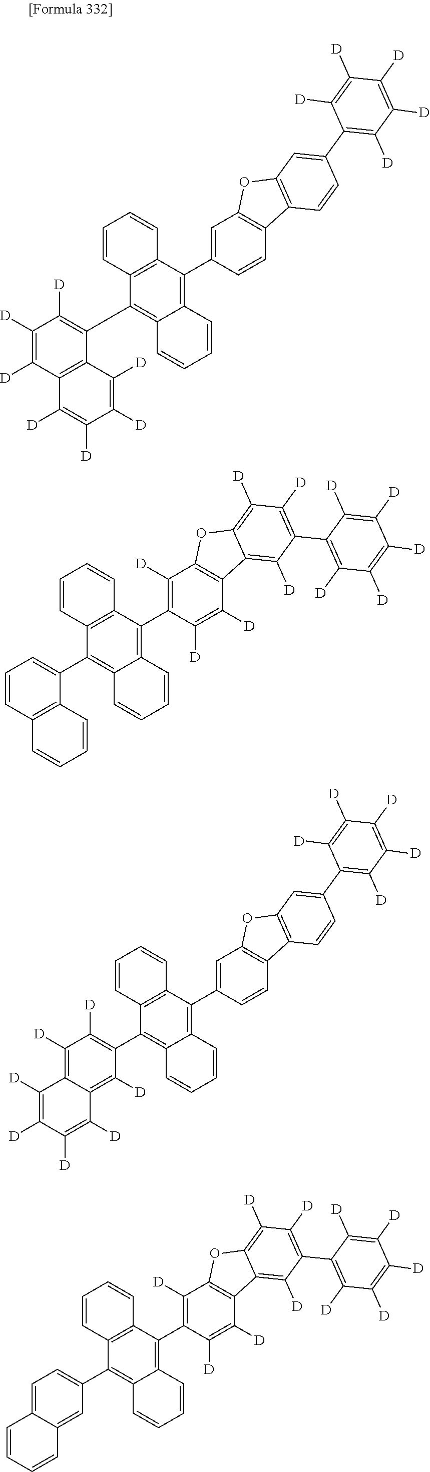

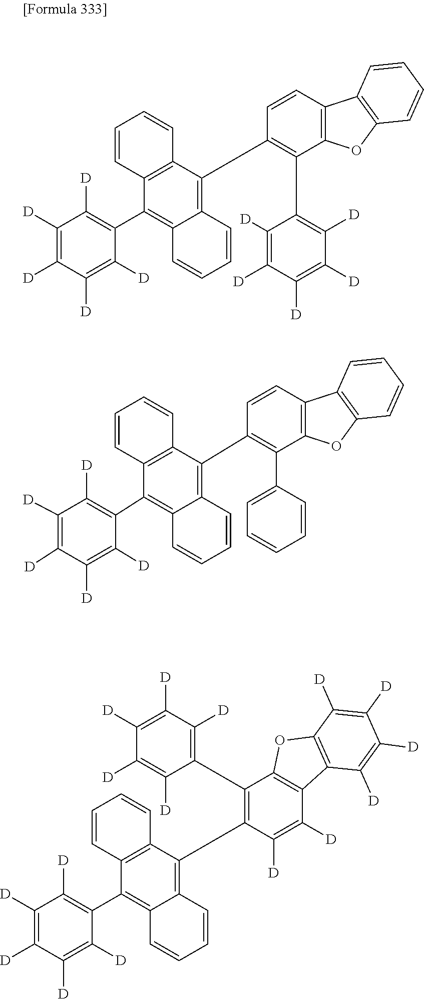 [SMILES: [2H]c1c([2H])c([2H])c(-c2c3ccccc3c(-c3ccc4c(oc5ccccc54)c3-c3c([2H])c([2H])c([2H])c([2H])c3[2H])c3ccccc23)c([2H])c1[2H] ; [2H]c1c([2H])c([2H])c(-c2c3ccccc3c(-c3ccc4c(oc5ccccc54)c3-c3ccccc3)c3ccccc23)c([2H])c1[2H] ; [2H]c1c([2H])c([2H])c(-c2c3ccccc3c(-c3c([2H])c([2H])c4c(oc5c([2H])c([2H])c([2H])c([2H])c54)c3-c3c([2H])c([2H])c([2H])c([2H])c3[2H])c3ccccc23)c([2H])c1[2H]]

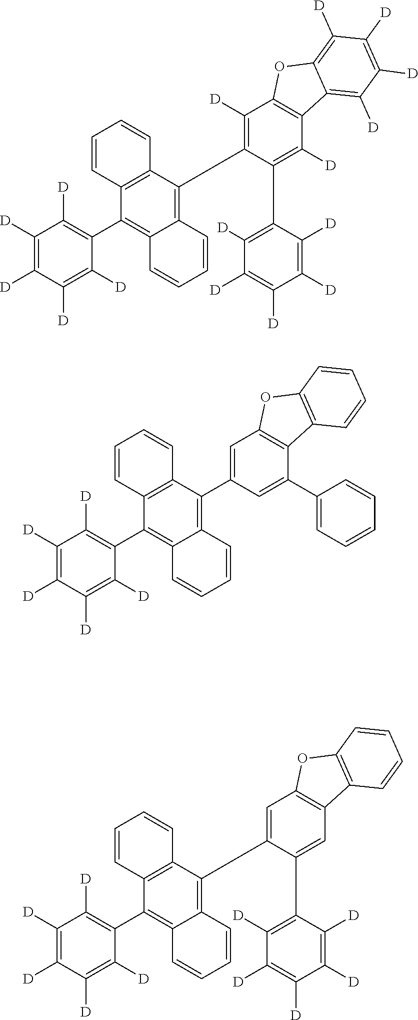 [SMILES: [2H]c1c([2H])c([2H])c(-c2c(-c3c4ccccc4c(-c4c([2H])c([2H])c([2H])c([2H])c4[2H])c4ccccc34)c([2H])c3oc4c([2H])c([2H])c([2H])c([2H])c4c3c2[2H])c([2H])c1[2H] ; [2H]c1c([2H])c([2H])c(-c2c3ccccc3c(-c3cc(-c4ccccc4)c4c(c3)oc3ccccc34)c3ccccc23)c([2H])c1[2H] ; [2H]c1c([2H])c([2H])c(-c2cc3c(cc2-c2c4ccccc4c(-c4c([2H])c([2H])c([2H])c([2H])c4[2H])c4ccccc24)oc2ccccc23)c([2H])c1[2H]]

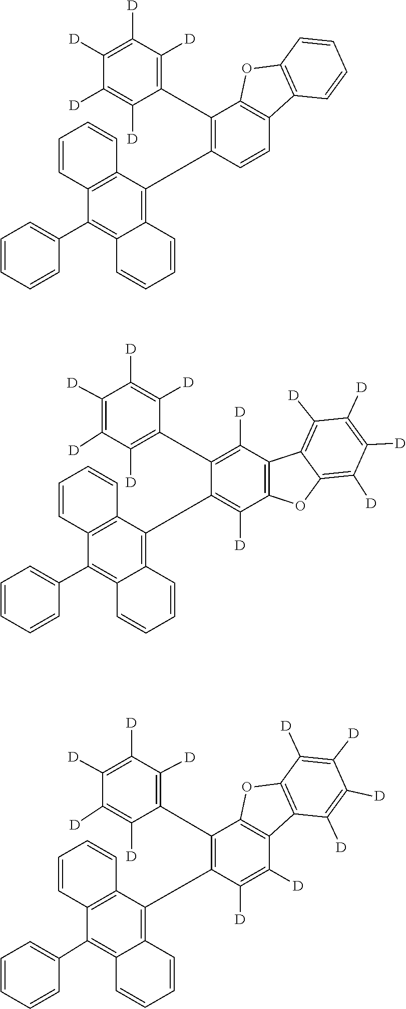 [SMILES: [2H]c1c([2H])c([2H])c(-c2c(-c3c4ccccc4c(-c4ccccc4)c4ccccc34)ccc3c2oc2ccccc23)c([2H])c1[2H] ; [2H]c1c([2H])c([2H])c(-c2c(-c3c4ccccc4c(-c4ccccc4)c4ccccc34)c([2H])c3oc4c([2H])c([2H])c([2H])c([2H])c4c3c2[2H])c([2H])c1[2H] ; [2H]c1c([2H])c([2H])c(-c2c(-c3c4ccccc4c(-c4ccccc4)c4ccccc34)c([2H])c([2H])c3c2oc2c([2H])c([2H])c([2H])c([2H])c23)c([2H])c1[2H]]

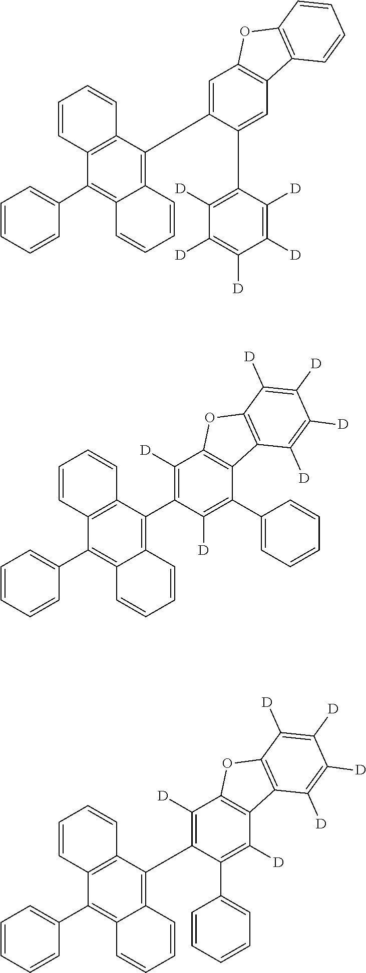 [SMILES: [2H]c1c([2H])c([2H])c(-c2cc3c(cc2-c2c4ccccc4c(-c4ccccc4)c4ccccc24)oc2ccccc23)c([2H])c1[2H] ; [2H]c1c([2H])c([2H])c2c(oc3c([2H])c(-c4c5ccccc5c(-c5ccccc5)c5ccccc45)c([2H])c(-c4ccccc4)c32)c1[2H] ; [2H]c1c([2H])c([2H])c2c(oc3c([2H])c(-c4c5ccccc5c(-c5ccccc5)c5ccccc45)c(-c4ccccc4)c([2H])c32)c1[2H]]

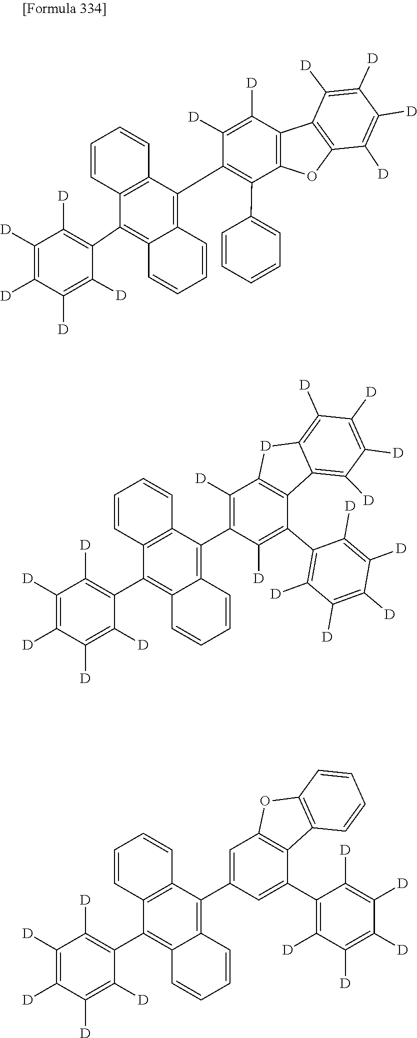

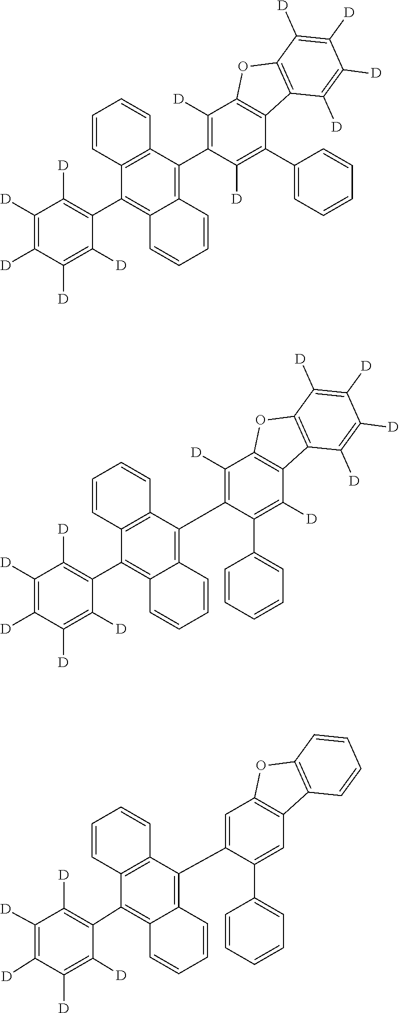 [SMILES: [2H]c1c([2H])c([2H])c(-c2c3ccccc3c(-c3c([2H])c(-c4ccccc4)c4c(oc5c([2H])c([2H])c([2H])c([2H])c54)c3[2H])c3ccccc23)c([2H])c1[2H] ; [2H]c1c([2H])c([2H])c(-c2c3ccccc3c(-c3c(-c4ccccc4)c([2H])c4c(oc5c([2H])c([2H])c([2H])c([2H])c54)c3[2H])c3ccccc23)c([2H])c1[2H] ; [2H]c1c([2H])c([2H])c(-c2c3ccccc3c(-c3cc4oc5ccccc5c4cc3-c3ccccc3)c3ccccc23)c([2H])c1[2H]]

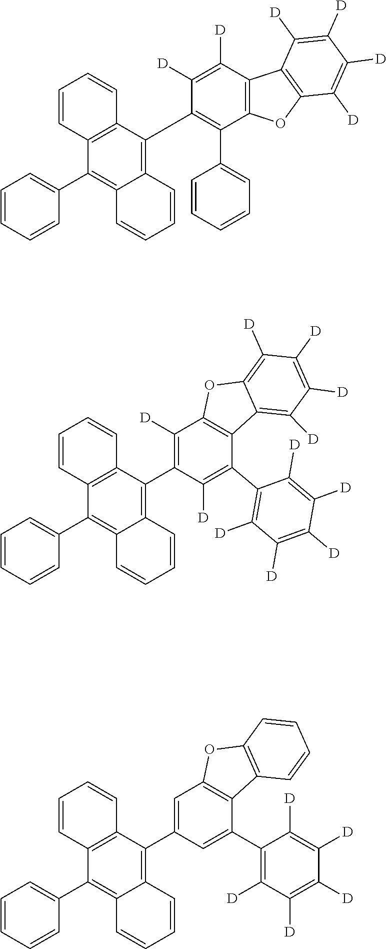 [SMILES: [2H]c1c([2H])c([2H])c2c(oc3c(-c4ccccc4)c(-c4c5ccccc5c(-c5ccccc5)c5ccccc45)c([2H])c([2H])c32)c1[2H] ; [2H]c1c([2H])c([2H])c(-c2c([2H])c(-c3c4ccccc4c(-c4ccccc4)c4ccccc34)c([2H])c3oc4c([2H])c([2H])c([2H])c([2H])c4c23)c([2H])c1[2H] ; [2H]c1c([2H])c([2H])c(-c2cc(-c3c4ccccc4c(-c4ccccc4)c4ccccc34)cc3oc4ccccc4c23)c([2H])c1[2H]]

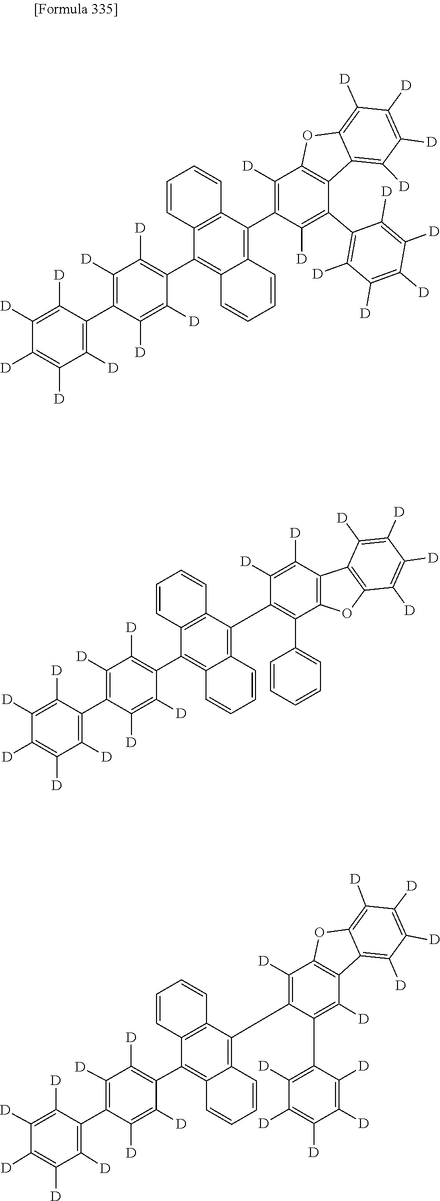

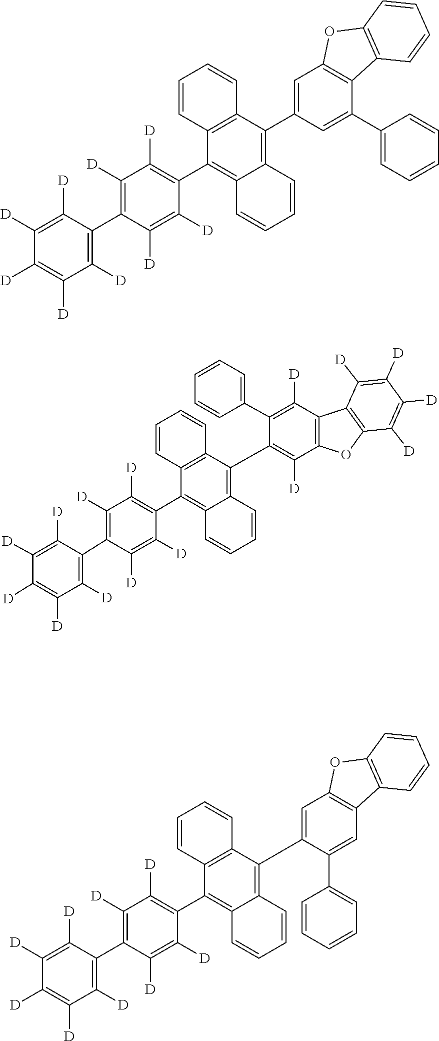 [SMILES: [2H]c1c([2H])c([2H])c(-c2c([2H])c([2H])c(-c3c4ccccc4c(-c4cc(-c5ccccc5)c5c(c4)oc4ccccc45)c4ccccc34)c([2H])c2[2H])c([2H])c1[2H] ; [2H]c1c([2H])c([2H])c(-c2c([2H])c([2H])c(-c3c4ccccc4c(-c4c(-c5ccccc5)c([2H])c5c(oc6c([2H])c([2H])c([2H])c([2H])c65)c4[2H])c4ccccc34)c([2H])c2[2H])c([2H])c1[2H] ; [2H]c1c([2H])c([2H])c(-c2c([2H])c([2H])c(-c3c4ccccc4c(-c4cc5oc6ccccc6c5cc4-c4ccccc4)c4ccccc34)c([2H])c2[2H])c([2H])c1[2H]]

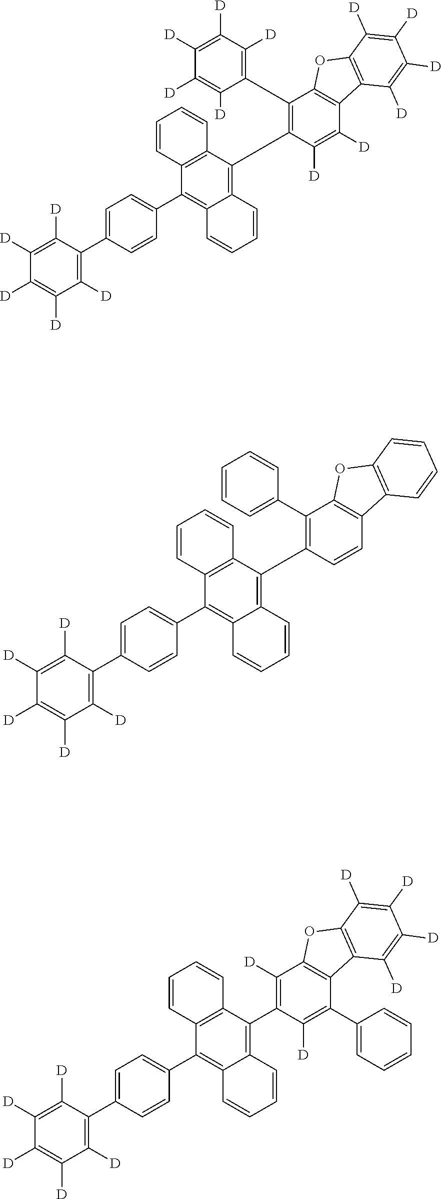 [SMILES: [2H]c1c([2H])c([2H])c(-c2ccc(-c3c4ccccc4c(-c4c([2H])c([2H])c5c(oc6c([2H])c([2H])c([2H])c([2H])c65)c4-c4c([2H])c([2H])c([2H])c([2H])c4[2H])c4ccccc34)cc2)c([2H])c1[2H] ; [2H]c1c([2H])c([2H])c(-c2ccc(-c3c4ccccc4c(-c4ccc5c(oc6ccccc65)c4-c4ccccc4)c4ccccc34)cc2)c([2H])c1[2H] ; [2H]c1c([2H])c([2H])c(-c2ccc(-c3c4ccccc4c(-c4c([2H])c(-c5ccccc5)c5c(oc6c([2H])c([2H])c([2H])c([2H])c65)c4[2H])c4ccccc34)cc2)c([2H])c1[2H]]

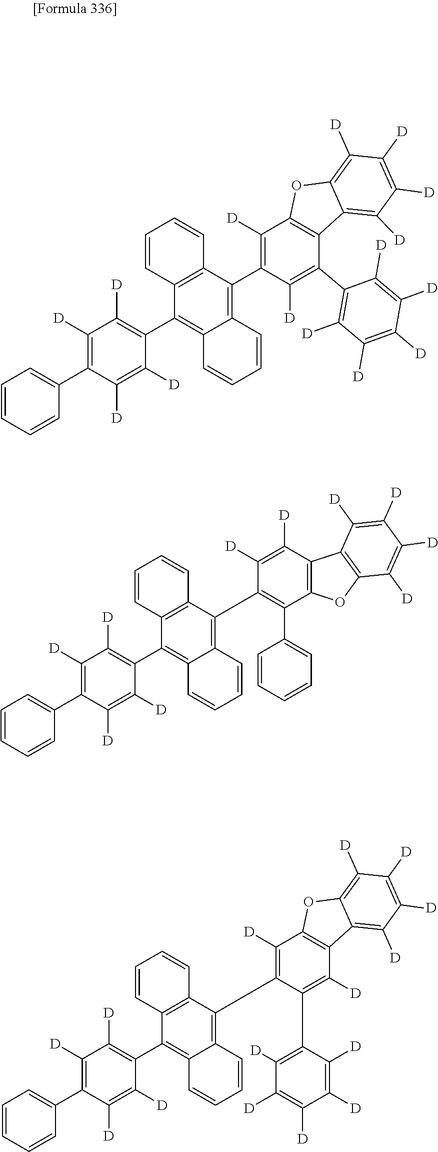 [SMILES: [2H]c1c([2H])c([2H])c(-c2c([2H])c(-c3c4ccccc4c(-c4c([2H])c([2H])c(-c5ccccc5)c([2H])c4[2H])c4ccccc34)c([2H])c3oc4c([2H])c([2H])c([2H])c([2H])c4c23)c([2H])c1[2H] ; [2H]c1c([2H])c(-c2c3ccccc3c(-c3c([2H])c([2H])c4c(oc5c([2H])c([2H])c([2H])c([2H])c54)c3-c3ccccc3)c3ccccc23)c([2H])c([2H])c1-c1ccccc1 ; [2H]c1c([2H])c([2H])c(-c2c(-c3c4ccccc4c(-c4c([2H])c([2H])c(-c5ccccc5)c([2H])c4[2H])c4ccccc34)c([2H])c3oc4c([2H])c([2H])c([2H])c([2H])c4c3c2[2H])c([2H])c1[2H]]

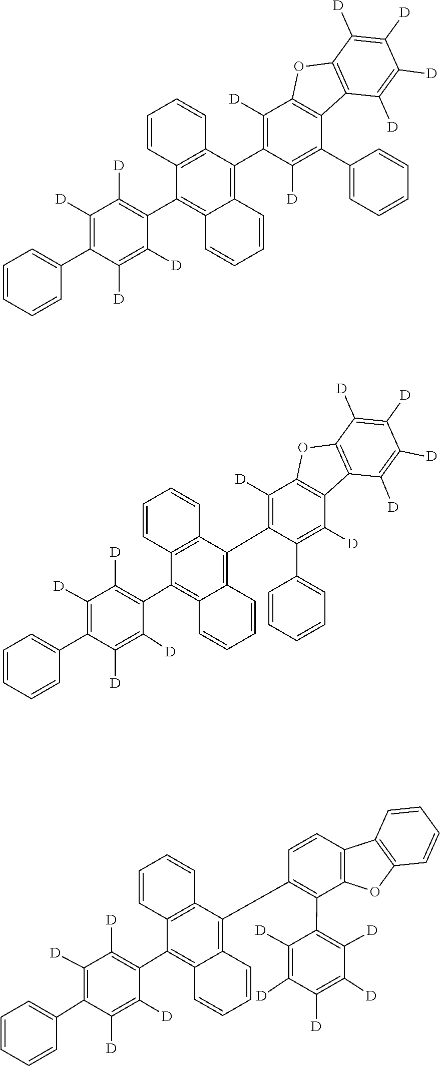 [SMILES: [2H]c1c([2H])c(-c2c3ccccc3c(-c3c([2H])c(-c4ccccc4)c4c(oc5c([2H])c([2H])c([2H])c([2H])c54)c3[2H])c3ccccc23)c([2H])c([2H])c1-c1ccccc1 ; [2H]c1c([2H])c(-c2c3ccccc3c(-c3c(-c4ccccc4)c([2H])c4c(oc5c([2H])c([2H])c([2H])c([2H])c54)c3[2H])c3ccccc23)c([2H])c([2H])c1-c1ccccc1 ; [2H]c1c([2H])c([2H])c(-c2c(-c3c4ccccc4c(-c4c([2H])c([2H])c(-c5ccccc5)c([2H])c4[2H])c4ccccc34)ccc3c2oc2ccccc23)c([2H])c1[2H]]

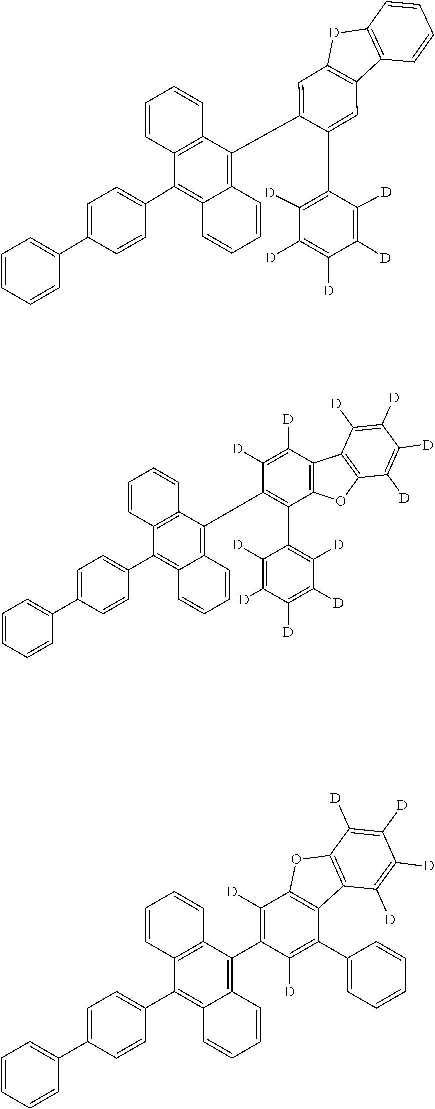

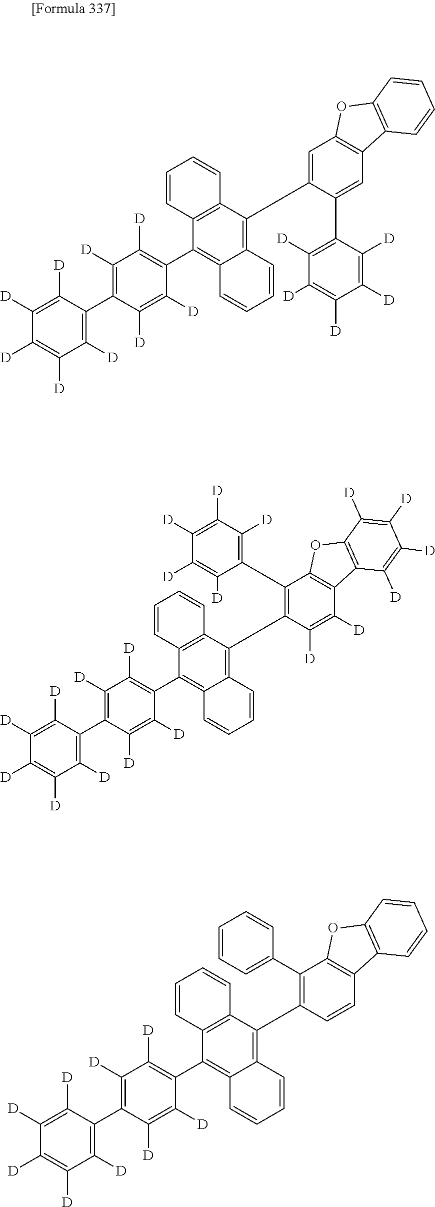 [SMILES: [2H]c1c([2H])c([2H])c(-c2cc3c(cc2-c2c4ccccc4c(-c4c([2H])c([2H])c(-c5c([2H])c([2H])c([2H])c([2H])c5[2H])c([2H])c4[2H])c4ccccc24)oc2ccccc23)c([2H])c1[2H] ; [2H]c1c([2H])c([2H])c(-c2c([2H])c([2H])c(-c3c4ccccc4c(-c4c([2H])c([2H])c5c(oc6c([2H])c([2H])c([2H])c([2H])c65)c4-c4c([2H])c([2H])c([2H])c([2H])c4[2H])c4ccccc34)c([2H])c2[2H])c([2H])c1[2H] ; [2H]c1c([2H])c([2H])c(-c2c([2H])c([2H])c(-c3c4ccccc4c(-c4ccc5c(oc6ccccc65)c4-c4ccccc4)c4ccccc34)c([2H])c2[2H])c([2H])c1[2H]]

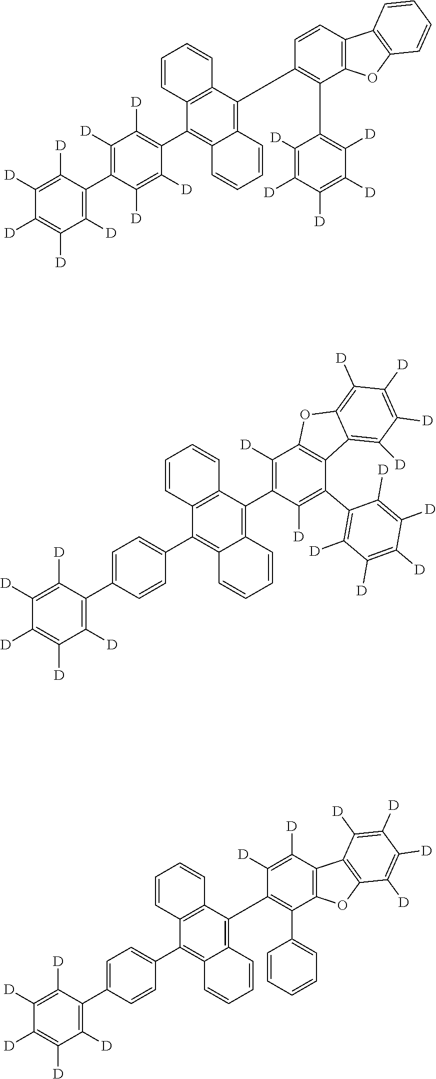 [SMILES: [2H]c1c([2H])c([2H])c(-c2c([2H])c([2H])c(-c3c4ccccc4c(-c4ccc5c(oc6ccccc65)c4-c4c([2H])c([2H])c([2H])c([2H])c4[2H])c4ccccc34)c([2H])c2[2H])c([2H])c1[2H] ; [2H]c1c([2H])c([2H])c(-c2ccc(-c3c4ccccc4c(-c4c([2H])c(-c5c([2H])c([2H])c([2H])c([2H])c5[2H])c5c(oc6c([2H])c([2H])c([2H])c([2H])c65)c4[2H])c4ccccc34)cc2)c([2H])c1[2H] ; [2H]c1c([2H])c([2H])c(-c2ccc(-c3c4ccccc4c(-c4c([2H])c([2H])c5c(oc6c([2H])c([2H])c([2H])c([2H])c65)c4-c4ccccc4)c4ccccc34)cc2)c([2H])c1[2H]]

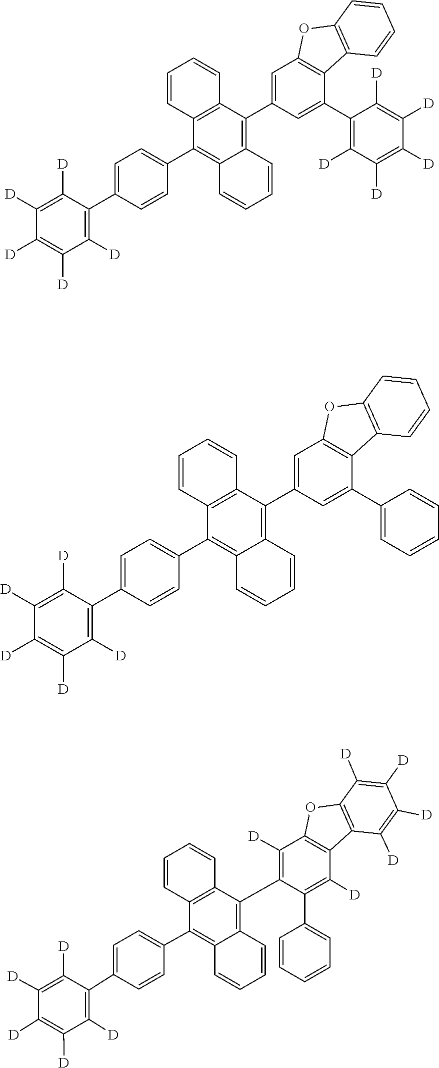 [SMILES: [2H]c1c([2H])c([2H])c(-c2ccc(-c3c4ccccc4c(-c4cc(-c5c([2H])c([2H])c([2H])c([2H])c5[2H])c5c(c4)oc4ccccc45)c4ccccc34)cc2)c([2H])c1[2H] ; [2H]c1c([2H])c([2H])c(-c2ccc(-c3c4ccccc4c(-c4cc(-c5ccccc5)c5c(c4)oc4ccccc45)c4ccccc34)cc2)c([2H])c1[2H] ; [2H]c1c([2H])c([2H])c(-c2ccc(-c3c4ccccc4c(-c4c(-c5ccccc5)c([2H])c5c(oc6c([2H])c([2H])c([2H])c([2H])c65)c4[2H])c4ccccc34)cc2)c([2H])c1[2H]]

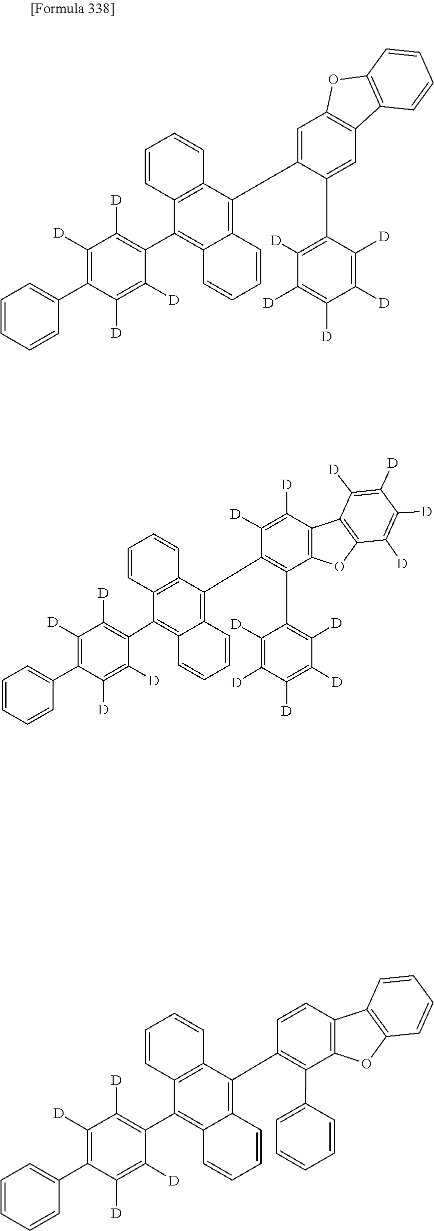 [SMILES: [2H]c1c([2H])c([2H])c(-c2cc3c(cc2-c2c4ccccc4c(-c4c([2H])c([2H])c(-c5ccccc5)c([2H])c4[2H])c4ccccc24)oc2ccccc23)c([2H])c1[2H] ; [2H]c1c([2H])c([2H])c(-c2c(-c3c4ccccc4c(-c4c([2H])c([2H])c(-c5ccccc5)c([2H])c4[2H])c4ccccc34)c([2H])c([2H])c3c2oc2c([2H])c([2H])c([2H])c([2H])c23)c([2H])c1[2H] ; [2H]c1c([2H])c(-c2c3ccccc3c(-c3ccc4c(oc5ccccc54)c3-c3ccccc3)c3ccccc23)c([2H])c([2H])c1-c1ccccc1]

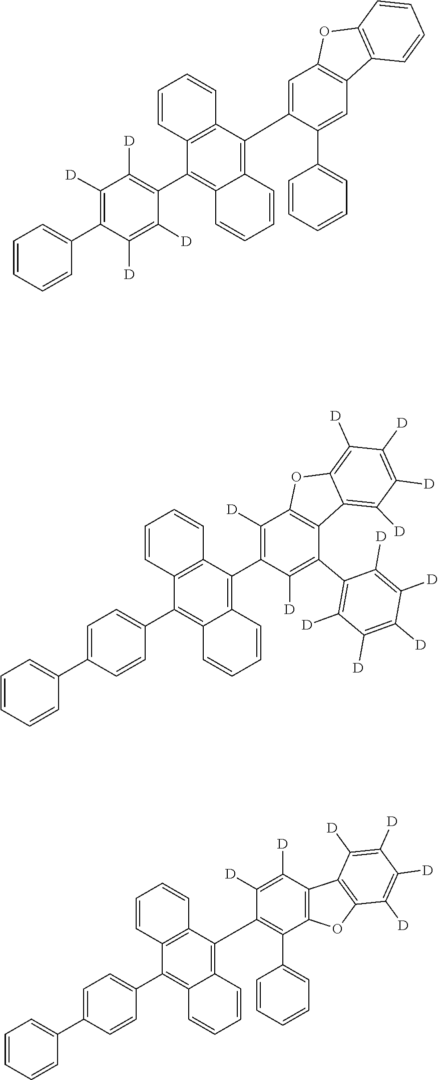 [SMILES: [2H]c1c([2H])c(-c2c3ccccc3c(-c3cc4oc5ccccc5c4cc3-c3ccccc3)c3ccccc23)c([2H])c([2H])c1-c1ccccc1 ; [2H]c1c([2H])c([2H])c(-c2c([2H])c(-c3c4ccccc4c(-c4ccc(-c5ccccc5)cc4)c4ccccc34)c([2H])c3oc4c([2H])c([2H])c([2H])c([2H])c4c23)c([2H])c1[2H] ; [2H]c1c([2H])c([2H])c2c(oc3c(-c4ccccc4)c(-c4c5ccccc5c(-c5ccc(-c6ccccc6)cc5)c5ccccc45)c([2H])c([2H])c32)c1[2H]]

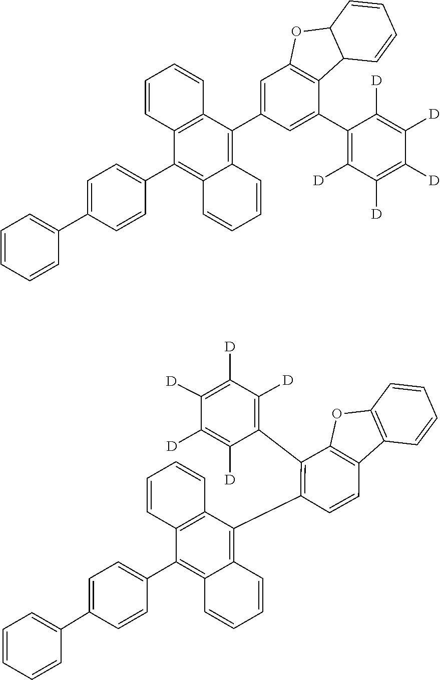 [SMILES: [2H]c1c([2H])c([2H])c(-c2cc(-c3c4ccccc4c(-c4ccc(-c5ccccc5)cc4)c4ccccc34)cc3c2C2C=CC=CC2O3)c([2H])c1[2H] ; [2H]c1c([2H])c([2H])c(-c2c(-c3c4ccccc4c(-c4ccc(-c5ccccc5)cc4)c4ccccc34)ccc3c2oc2ccccc23)c([2H])c1[2H]]

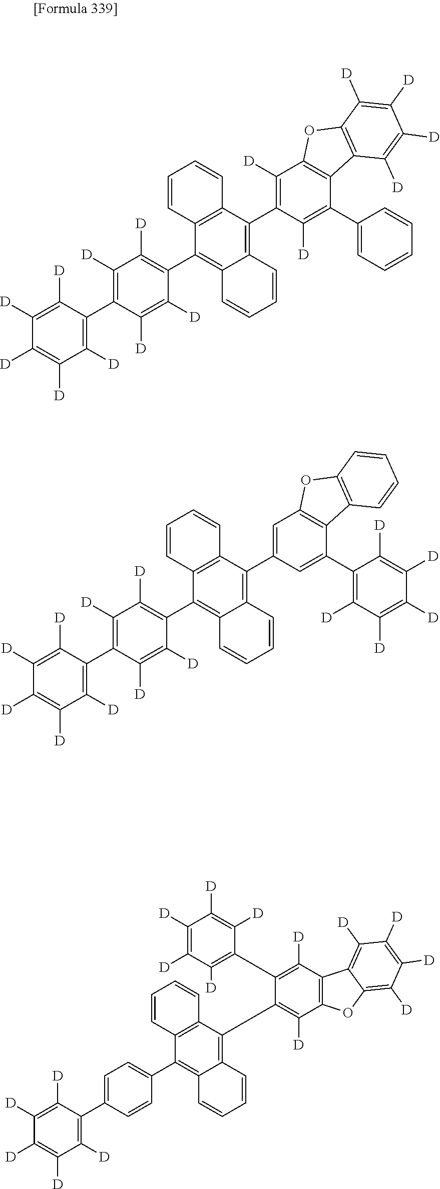 [SMILES: [2H]c1c([2H])c([2H])c(-c2c([2H])c([2H])c(-c3c4ccccc4c(-c4c([2H])c(-c5ccccc5)c5c(oc6c([2H])c([2H])c([2H])c([2H])c65)c4[2H])c4ccccc34)c([2H])c2[2H])c([2H])c1[2H] ; [2H]c1c([2H])c([2H])c(-c2c([2H])c([2H])c(-c3c4ccccc4c(-c4cc(-c5c([2H])c([2H])c([2H])c([2H])c5[2H])c5c(c4)oc4ccccc45)c4ccccc34)c([2H])c2[2H])c([2H])c1[2H] ; [2H]c1c([2H])c([2H])c(-c2ccc(-c3c4ccccc4c(-c4c(-c5c([2H])c([2H])c([2H])c([2H])c5[2H])c([2H])c5c(oc6c([2H])c([2H])c([2H])c([2H])c65)c4[2H])c4ccccc34)cc2)c([2H])c1[2H]]

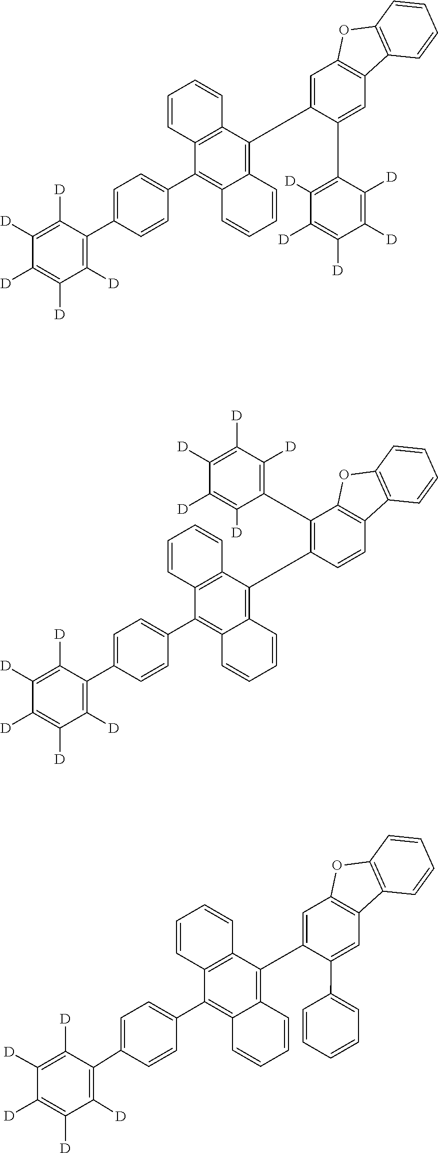 [SMILES: [2H]c1c([2H])c([2H])c(-c2ccc(-c3c4ccccc4c(-c4cc5oc6ccccc6c5cc4-c4c([2H])c([2H])c([2H])c([2H])c4[2H])c4ccccc34)cc2)c([2H])c1[2H] ; [2H]c1c([2H])c([2H])c(-c2ccc(-c3c4ccccc4c(-c4ccc5c(oc6ccccc65)c4-c4c([2H])c([2H])c([2H])c([2H])c4[2H])c4ccccc34)cc2)c([2H])c1[2H] ; [2H]c1c([2H])c([2H])c(-c2ccc(-c3c4ccccc4c(-c4cc5oc6ccccc6c5cc4-c4ccccc4)c4ccccc34)cc2)c([2H])c1[2H]]

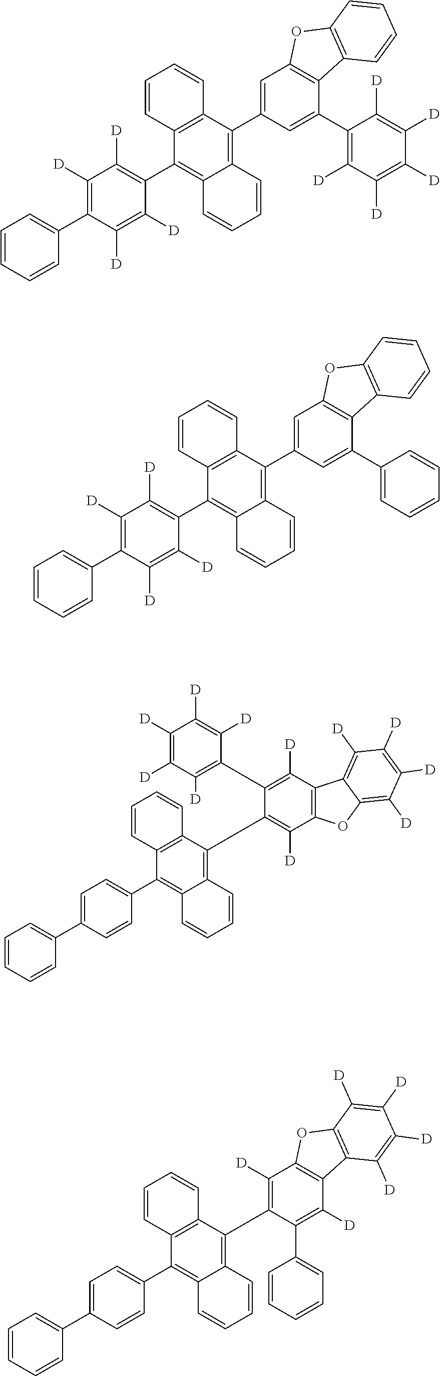 [SMILES: [2H]c1c([2H])c([2H])c(-c2cc(-c3c4ccccc4c(-c4c([2H])c([2H])c(-c5ccccc5)c([2H])c4[2H])c4ccccc34)cc3oc4ccccc4c23)c([2H])c1[2H] ; [2H]c1c([2H])c(-c2c3ccccc3c(-c3cc(-c4ccccc4)c4c(c3)oc3ccccc34)c3ccccc23)c([2H])c([2H])c1-c1ccccc1 ; [2H]c1c([2H])c([2H])c(-c2c(-c3c4ccccc4c(-c4ccc(-c5ccccc5)cc4)c4ccccc34)c([2H])c3oc4c([2H])c([2H])c([2H])c([2H])c4c3c2[2H])c([2H])c1[2H] ; [2H]c1c([2H])c([2H])c2c(oc3c([2H])c(-c4c5ccccc5c(-c5ccc(-c6ccccc6)cc5)c5ccccc45)c(-c4ccccc4)c([2H])c32)c1[2H]]

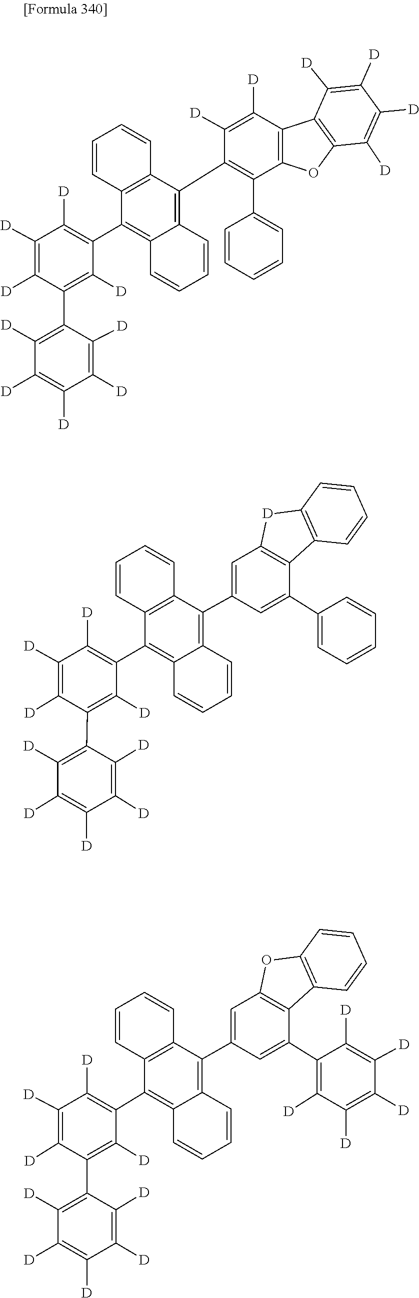

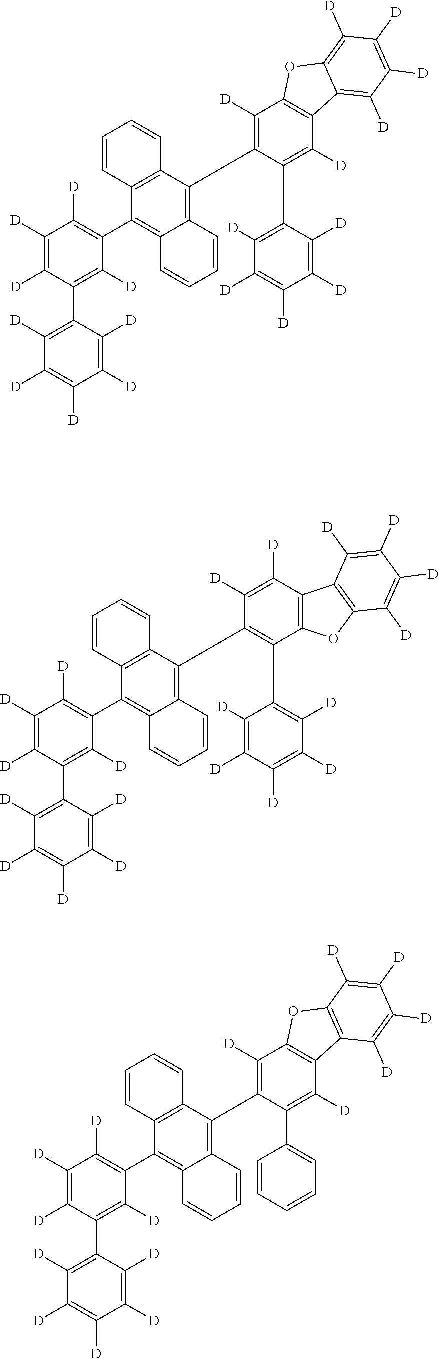

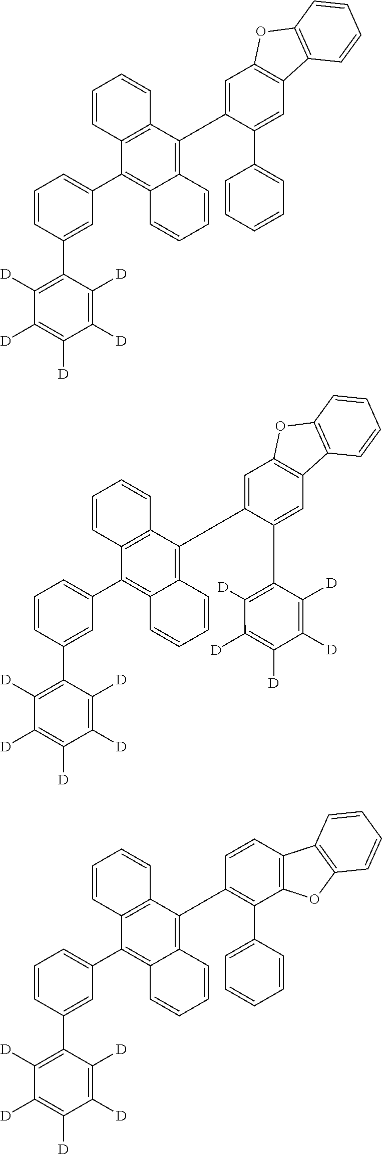 [SMILES: [2H]c1c([2H])c([2H])c(-c2cccc(-c3c4ccccc4c(-c4cc5oc6ccccc6c5cc4-c4ccccc4)c4ccccc34)c2)c([2H])c1[2H] ; [2H]c1c([2H])c([2H])c(-c2cccc(-c3c4ccccc4c(-c4cc5oc6ccccc6c5cc4-c4c([2H])c([2H])c([2H])c([2H])c4[2H])c4ccccc34)c2)c([2H])c1[2H] ; [2H]c1c([2H])c([2H])c(-c2cccc(-c3c4ccccc4c(-c4ccc5c(oc6ccccc65)c4-c4ccccc4)c4ccccc34)c2)c([2H])c1[2H]]

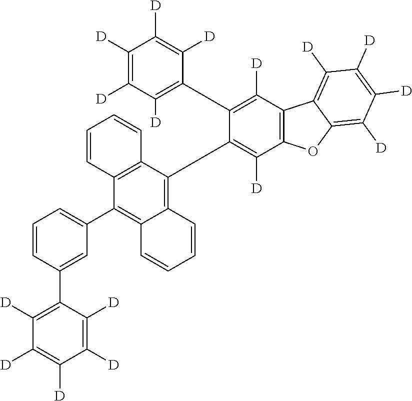 [SMILES: [2H]c1c([2H])c([2H])c(-c2cccc(-c3c4ccccc4c(-c4c(-c5c([2H])c([2H])c([2H])c([2H])c5[2H])c([2H])c5c(oc6c([2H])c([2H])c([2H])c([2H])c65)c4[2H])c4ccccc34)c2)c([2H])c1[2H]]

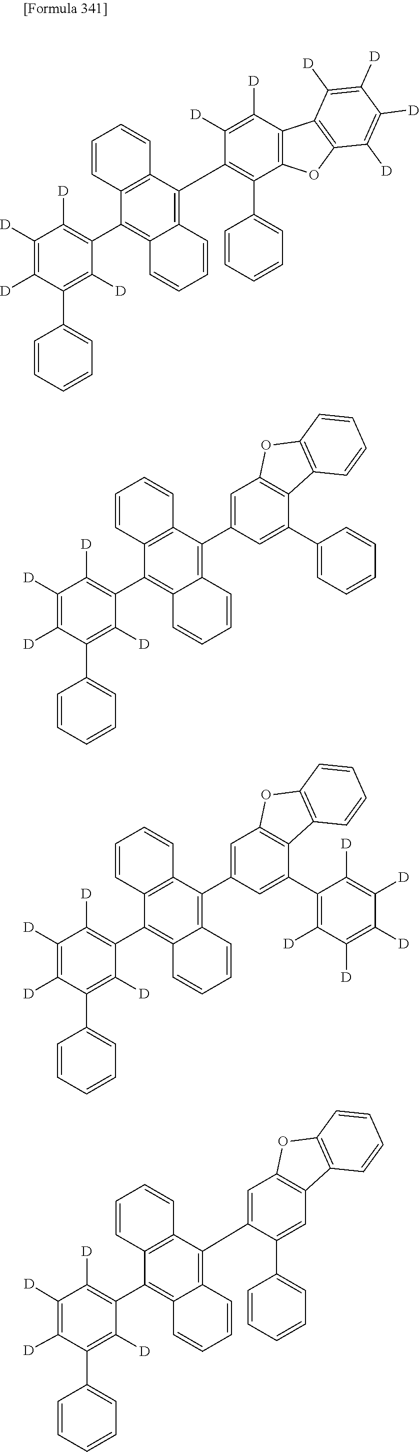 [SMILES: [2H]c1c([2H])c(-c2ccccc2)c([2H])c(-c2c3ccccc3c(-c3c([2H])c([2H])c4c(oc5c([2H])c([2H])c([2H])c([2H])c54)c3-c3ccccc3)c3ccccc23)c1[2H] ; [2H]c1c([2H])c(-c2ccccc2)c([2H])c(-c2c3ccccc3c(-c3cc(-c4ccccc4)c4c(c3)oc3ccccc34)c3ccccc23)c1[2H] ; [2H]c1c([2H])c([2H])c(-c2cc(-c3c4ccccc4c(-c4c([2H])c([2H])c([2H])c(-c5ccccc5)c4[2H])c4ccccc34)cc3oc4ccccc4c23)c([2H])c1[2H] ; [2H]c1c([2H])c(-c2ccccc2)c([2H])c(-c2c3ccccc3c(-c3cc4oc5ccccc5c4cc3-c3ccccc3)c3ccccc23)c1[2H]]

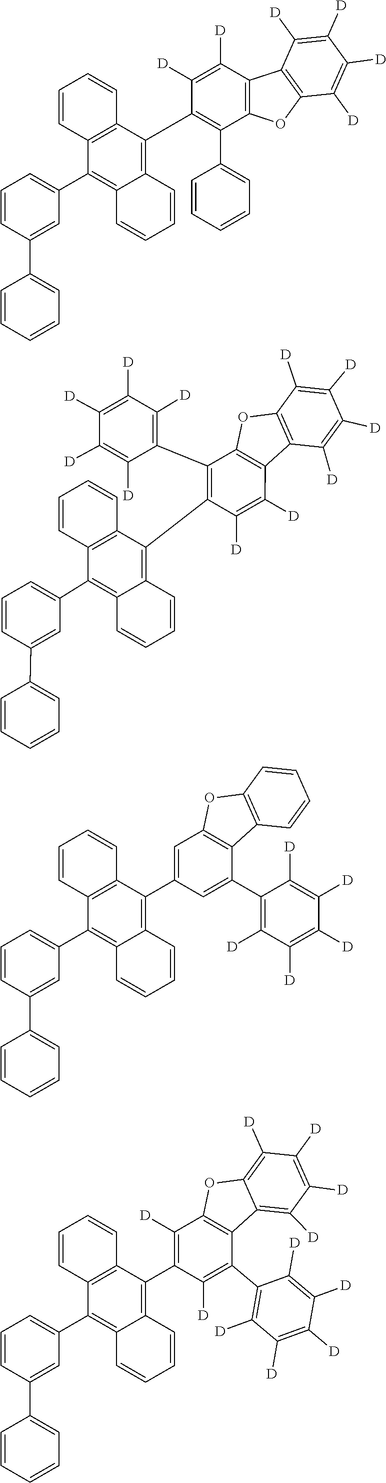 [SMILES: [2H]c1c([2H])c([2H])c2c(oc3c(-c4ccccc4)c(-c4c5ccccc5c(-c5cccc(-c6ccccc6)c5)c5ccccc45)c([2H])c([2H])c32)c1[2H] ; [2H]c1c([2H])c([2H])c(-c2c(-c3c4ccccc4c(-c4cccc(-c5ccccc5)c4)c4ccccc34)c([2H])c([2H])c3c2oc2c([2H])c([2H])c([2H])c([2H])c23)c([2H])c1[2H] ; [2H]c1c([2H])c([2H])c(-c2cc(-c3c4ccccc4c(-c4cccc(-c5ccccc5)c4)c4ccccc34)cc3oc4ccccc4c23)c([2H])c1[2H] ; [2H]c1c([2H])c([2H])c(-c2c([2H])c(-c3c4ccccc4c(-c4cccc(-c5ccccc5)c4)c4ccccc34)c([2H])c3oc4c([2H])c([2H])c([2H])c([2H])c4c23)c([2H])c1[2H]]

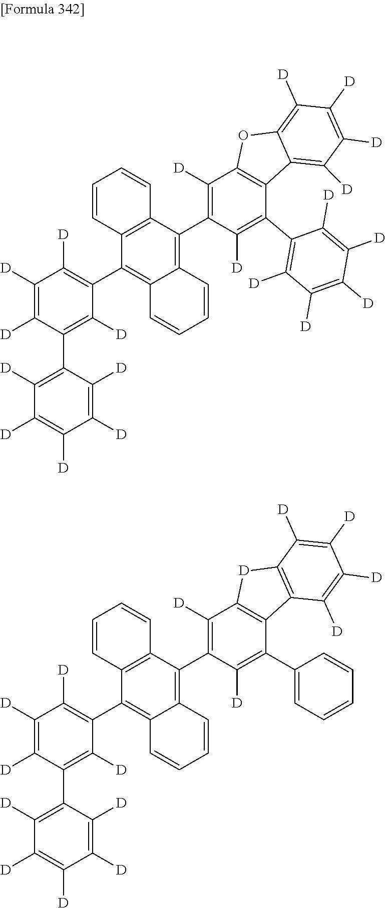

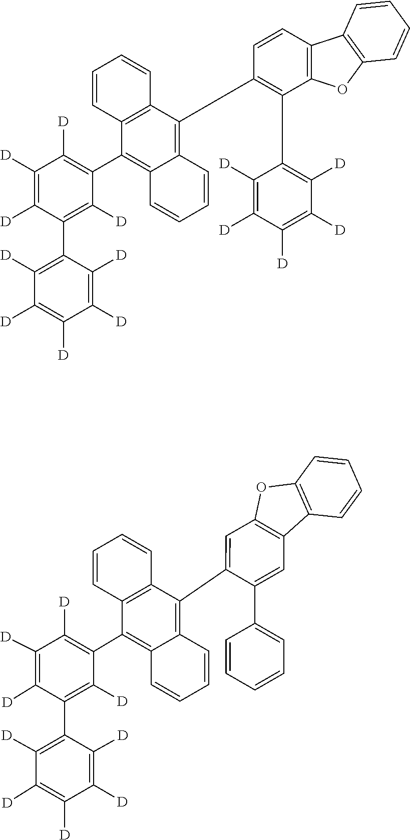 [SMILES: [2H]c1c([2H])c([2H])c(-c2c([2H])c([2H])c([2H])c(-c3c4ccccc4c(-c4ccc5c(oc6ccccc65)c4-c4c([2H])c([2H])c([2H])c([2H])c4[2H])c4ccccc34)c2[2H])c([2H])c1[2H] ; [2H]c1c([2H])c([2H])c(-c2c([2H])c([2H])c([2H])c(-c3c4ccccc4c(-c4cc5oc6ccccc6c5cc4-c4ccccc4)c4ccccc34)c2[2H])c([2H])c1[2H]]

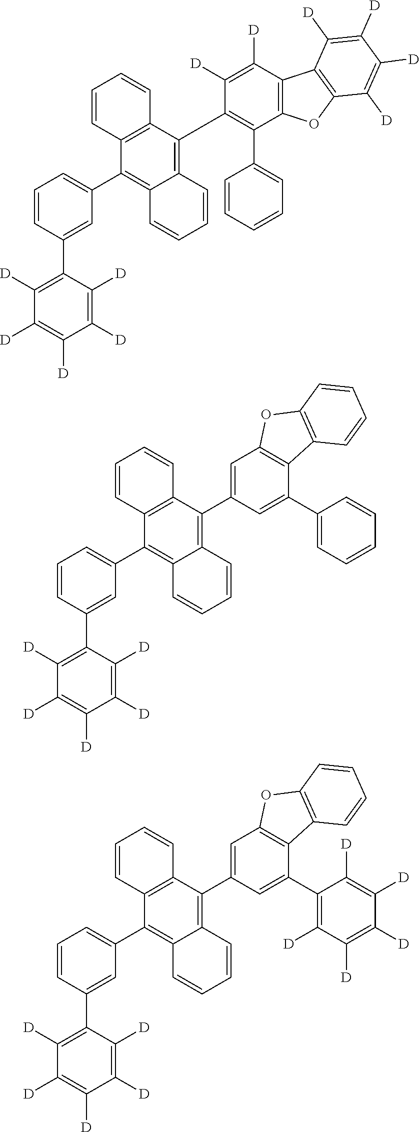 [SMILES: [2H]c1c([2H])c([2H])c(-c2cccc(-c3c4ccccc4c(-c4c([2H])c([2H])c5c(oc6c([2H])c([2H])c([2H])c([2H])c65)c4-c4ccccc4)c4ccccc34)c2)c([2H])c1[2H] ; [2H]c1c([2H])c([2H])c(-c2cccc(-c3c4ccccc4c(-c4cc(-c5ccccc5)c5c(c4)oc4ccccc45)c4ccccc34)c2)c([2H])c1[2H] ; [2H]c1c([2H])c([2H])c(-c2cccc(-c3c4ccccc4c(-c4cc(-c5c([2H])c([2H])c([2H])c([2H])c5[2H])c5c(c4)oc4ccccc45)c4ccccc34)c2)c([2H])c1[2H]]

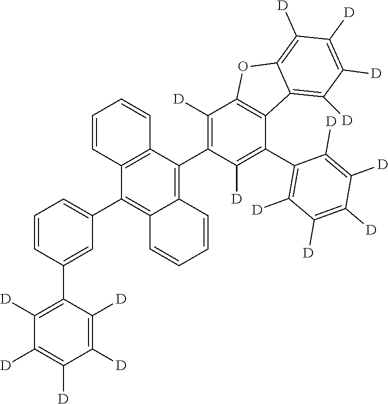 [SMILES: [2H]c1c([2H])c([2H])c(-c2cccc(-c3c4ccccc4c(-c4c([2H])c(-c5c([2H])c([2H])c([2H])c([2H])c5[2H])c5c(oc6c([2H])c([2H])c([2H])c([2H])c65)c4[2H])c4ccccc34)c2)c([2H])c1[2H]]

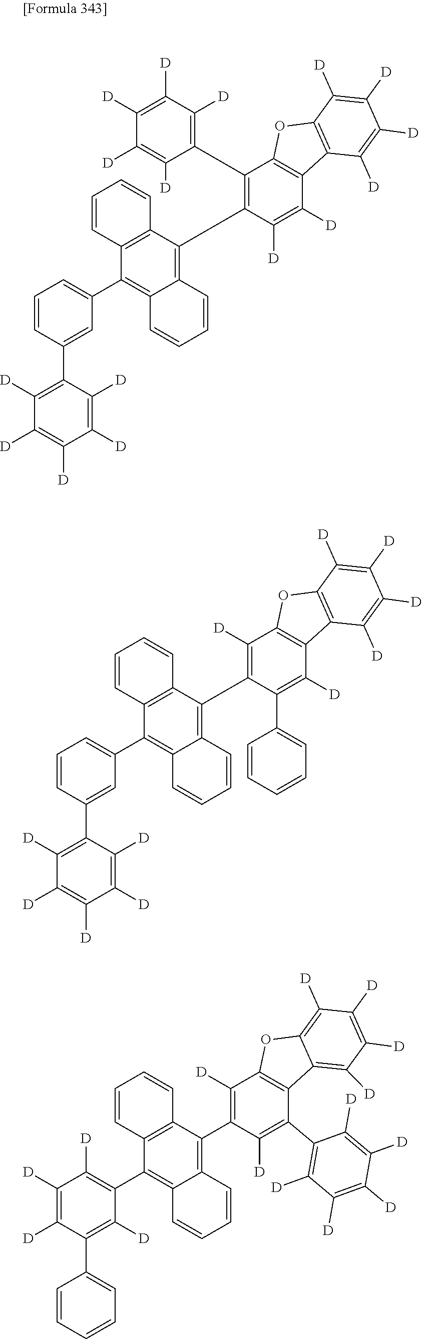 [SMILES: [2H]c1c([2H])c([2H])c(-c2cccc(-c3c4ccccc4c(-c4c([2H])c([2H])c5c(oc6c([2H])c([2H])c([2H])c([2H])c65)c4-c4c([2H])c([2H])c([2H])c([2H])c4[2H])c4ccccc34)c2)c([2H])c1[2H] ; [2H]c1c([2H])c([2H])c(-c2cccc(-c3c4ccccc4c(-c4c(-c5ccccc5)c([2H])c5c(oc6c([2H])c([2H])c([2H])c([2H])c65)c4[2H])c4ccccc34)c2)c([2H])c1[2H] ; [2H]c1c([2H])c([2H])c(-c2c([2H])c(-c3c4ccccc4c(-c4c([2H])c([2H])c([2H])c(-c5ccccc5)c4[2H])c4ccccc34)c([2H])c3oc4c([2H])c([2H])c([2H])c([2H])c4c23)c([2H])c1[2H]]

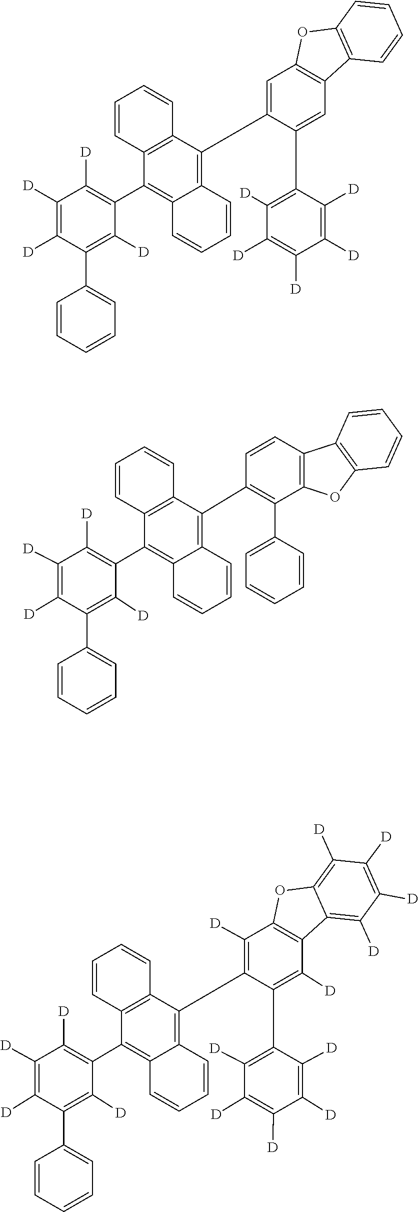 [SMILES: [2H]c1c([2H])c([2H])c(-c2cc3c(cc2-c2c4ccccc4c(-c4c([2H])c([2H])c([2H])c(-c5ccccc5)c4[2H])c4ccccc24)oc2ccccc23)c([2H])c1[2H] ; [2H]c1c([2H])c(-c2ccccc2)c([2H])c(-c2c3ccccc3c(-c3ccc4c(oc5ccccc54)c3-c3ccccc3)c3ccccc23)c1[2H] ; [2H]c1c([2H])c([2H])c(-c2c(-c3c4ccccc4c(-c4c([2H])c([2H])c([2H])c(-c5ccccc5)c4[2H])c4ccccc34)c([2H])c3oc4c([2H])c([2H])c([2H])c([2H])c4c3c2[2H])c([2H])c1[2H]]

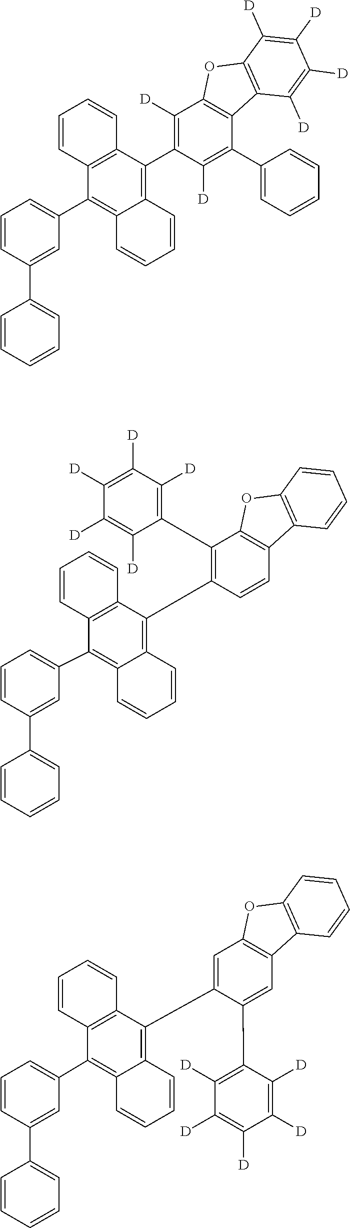 [SMILES: [2H]c1c([2H])c([2H])c2c(oc3c([2H])c(-c4c5ccccc5c(-c5cccc(-c6ccccc6)c5)c5ccccc45)c([2H])c(-c4ccccc4)c32)c1[2H] ; [2H]c1c([2H])c([2H])c(-c2c(-c3c4ccccc4c(-c4cccc(-c5ccccc5)c4)c4ccccc34)ccc3c2oc2ccccc23)c([2H])c1[2H] ; [2H]c1c([2H])c([2H])c(-c2cc3c(cc2-c2c4ccccc4c(-c4cccc(-c5ccccc5)c4)c4ccccc24)oc2ccccc23)c([2H])c1[2H]]

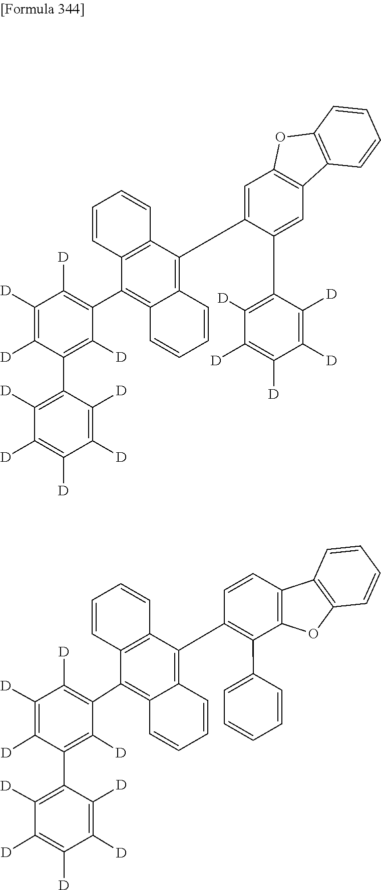 [SMILES: [2H]c1c([2H])c([2H])c(-c2cc3c(cc2-c2c4ccccc4c(-c4c([2H])c([2H])c([2H])c(-c5c([2H])c([2H])c([2H])c([2H])c5[2H])c4[2H])c4ccccc24)oc2ccccc23)c([2H])c1[2H] ; [2H]c1c([2H])c([2H])c(-c2c([2H])c([2H])c([2H])c(-c3c4ccccc4c(-c4ccc5c(oc6ccccc65)c4-c4ccccc4)c4ccccc34)c2[2H])c([2H])c1[2H]]

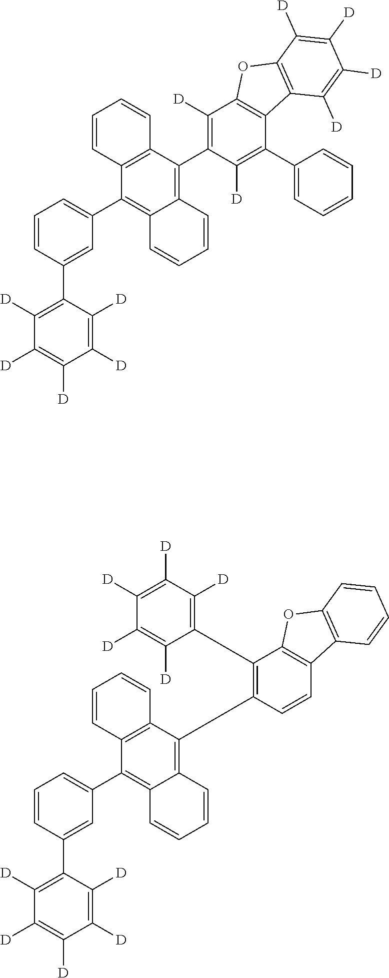 [SMILES: [2H]c1c([2H])c([2H])c(-c2cccc(-c3c4ccccc4c(-c4c([2H])c(-c5ccccc5)c5c(oc6c([2H])c([2H])c([2H])c([2H])c65)c4[2H])c4ccccc34)c2)c([2H])c1[2H] ; [2H]c1c([2H])c([2H])c(-c2cccc(-c3c4ccccc4c(-c4ccc5c(oc6ccccc65)c4-c4c([2H])c([2H])c([2H])c([2H])c4[2H])c4ccccc34)c2)c([2H])c1[2H]]

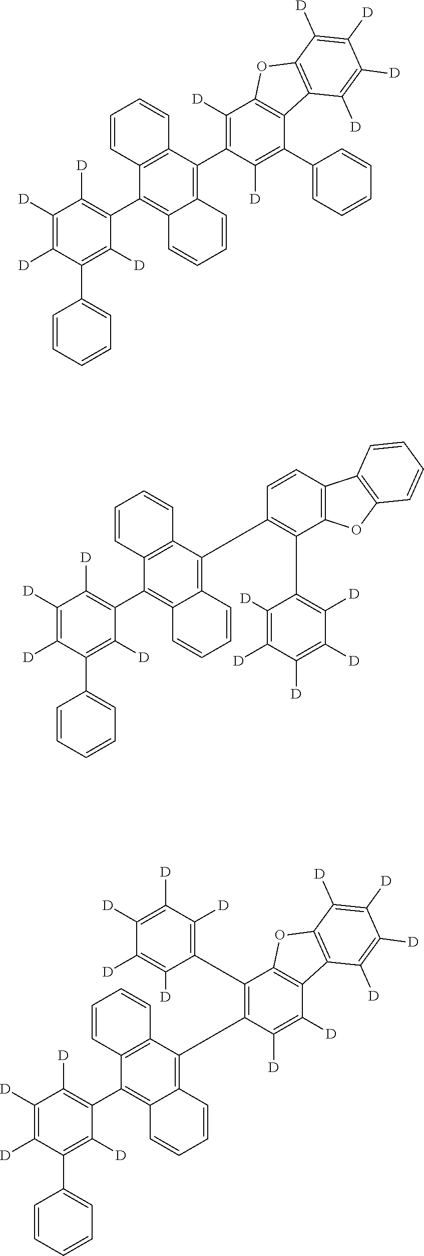 [SMILES: [2H]c1c([2H])c(-c2ccccc2)c([2H])c(-c2c3ccccc3c(-c3c([2H])c(-c4ccccc4)c4c(oc5c([2H])c([2H])c([2H])c([2H])c54)c3[2H])c3ccccc23)c1[2H] ; [2H]c1c([2H])c([2H])c(-c2c(-c3c4ccccc4c(-c4c([2H])c([2H])c([2H])c(-c5ccccc5)c4[2H])c4ccccc34)ccc3c2oc2ccccc23)c([2H])c1[2H] ; [2H]c1c([2H])c([2H])c(-c2c(-c3c4ccccc4c(-c4c([2H])c([2H])c([2H])c(-c5ccccc5)c4[2H])c4ccccc34)c([2H])c([2H])c3c2oc2c([2H])c([2H])c([2H])c([2H])c23)c([2H])c1[2H]]

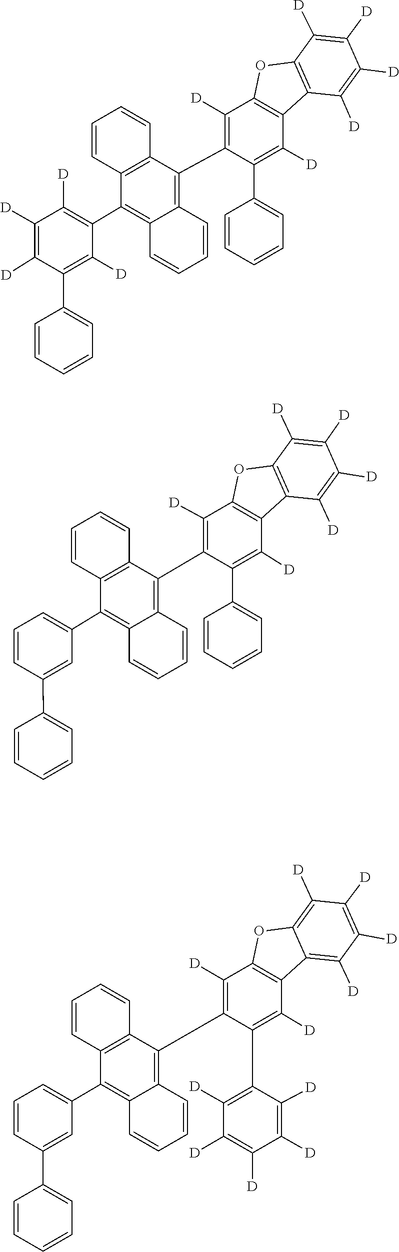 [SMILES: [2H]c1c([2H])c(-c2ccccc2)c([2H])c(-c2c3ccccc3c(-c3c(-c4ccccc4)c([2H])c4c(oc5c([2H])c([2H])c([2H])c([2H])c54)c3[2H])c3ccccc23)c1[2H] ; [2H]c1c([2H])c([2H])c2c(oc3c([2H])c(-c4c5ccccc5c(-c5cccc(-c6ccccc6)c5)c5ccccc45)c(-c4ccccc4)c([2H])c32)c1[2H] ; [2H]c1c([2H])c([2H])c(-c2c(-c3c4ccccc4c(-c4cccc(-c5ccccc5)c4)c4ccccc34)c([2H])c3oc4c([2H])c([2H])c([2H])c([2H])c4c3c2[2H])c([2H])c1[2H]]

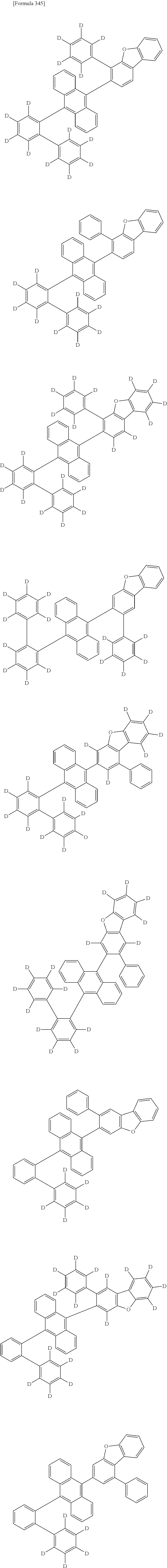

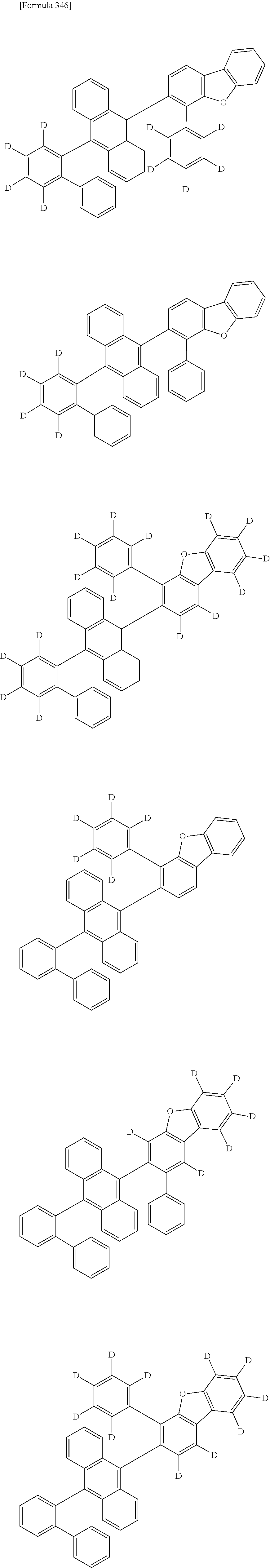

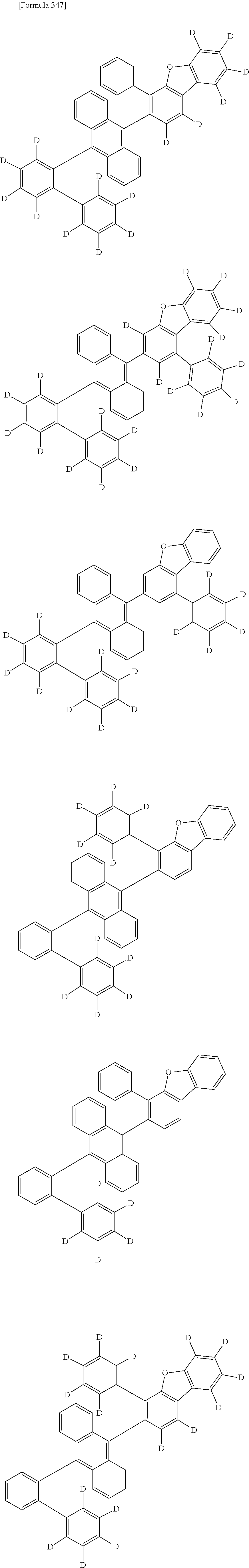

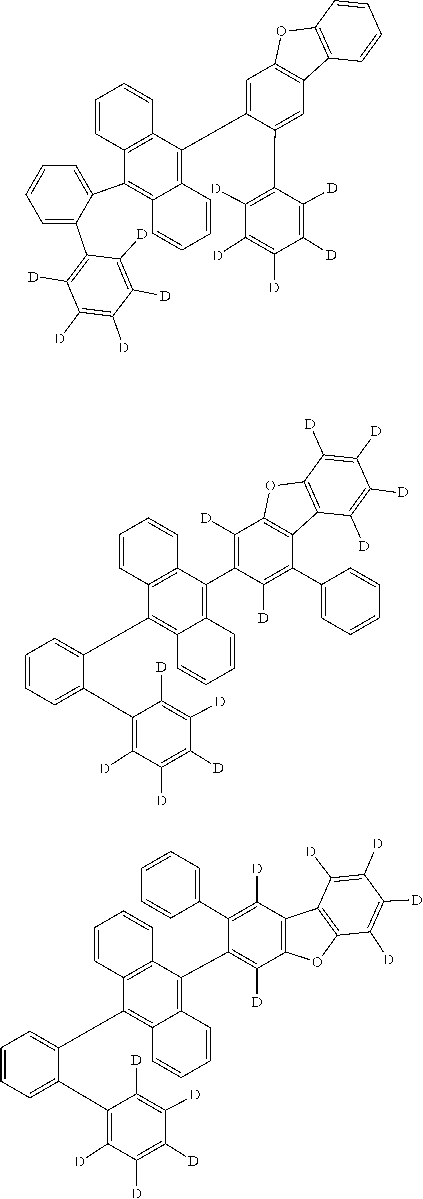 [SMILES: [2H]c1c([2H])c([2H])c(-c2ccccc2-c2c3ccccc3c(-c3cc4oc5ccccc5c4cc3-c3c([2H])c([2H])c([2H])c([2H])c3[2H])c3ccccc23)c([2H])c1[2H] ; [2H]c1c([2H])c([2H])c(-c2ccccc2-c2c3ccccc3c(-c3c([2H])c(-c4ccccc4)c4c(oc5c([2H])c([2H])c([2H])c([2H])c54)c3[2H])c3ccccc23)c([2H])c1[2H] ; [2H]c1c([2H])c([2H])c(-c2ccccc2-c2c3ccccc3c(-c3c(-c4ccccc4)c([2H])c4c(oc5c([2H])c([2H])c([2H])c([2H])c54)c3[2H])c3ccccc23)c([2H])c1[2H]]

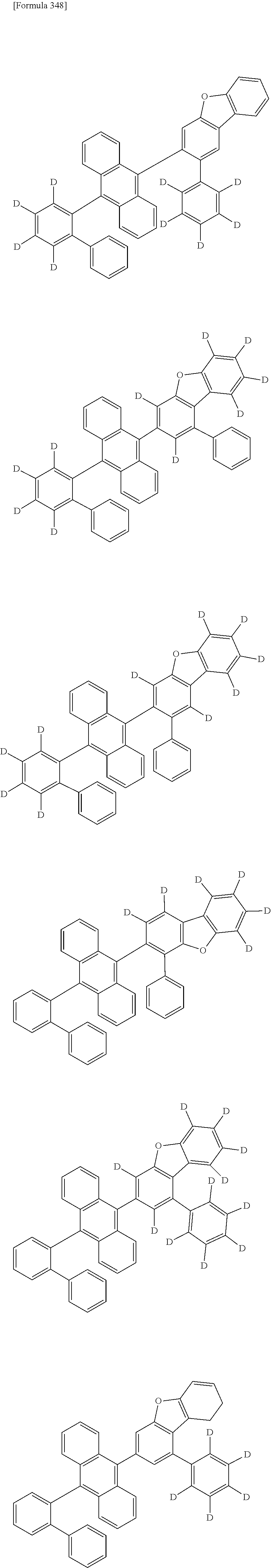

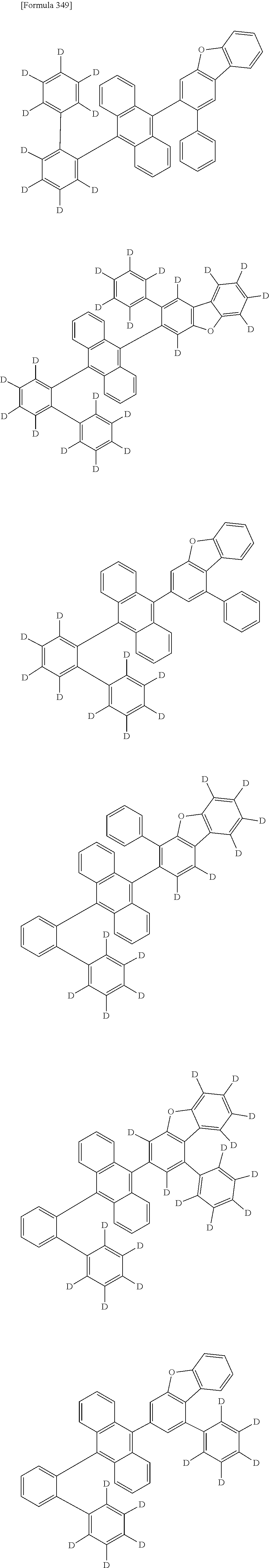

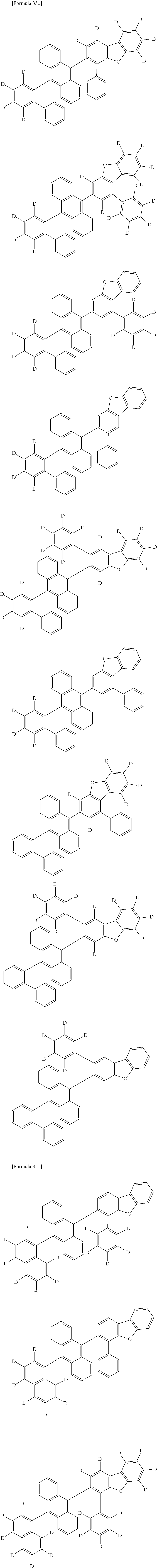

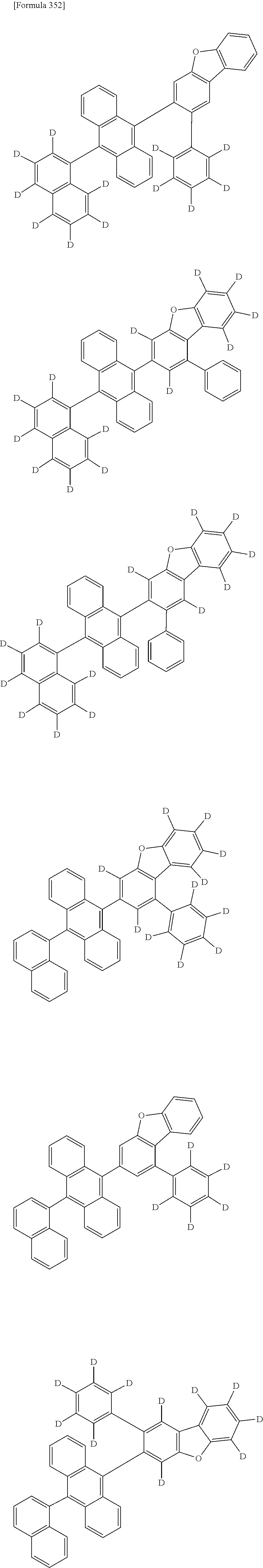

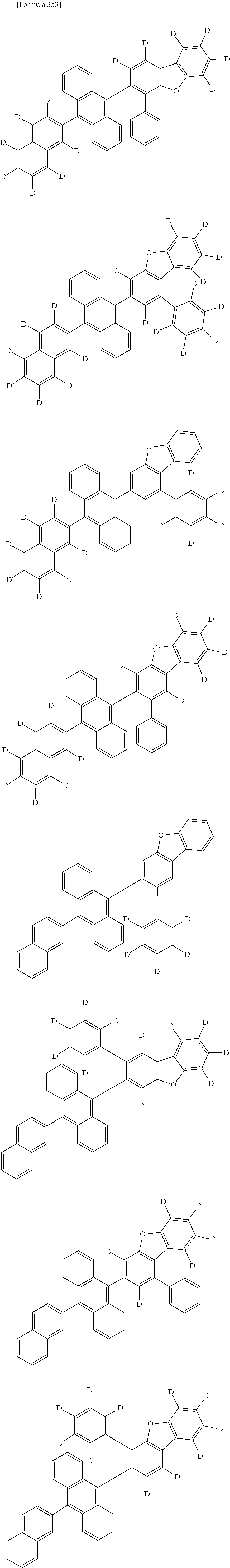

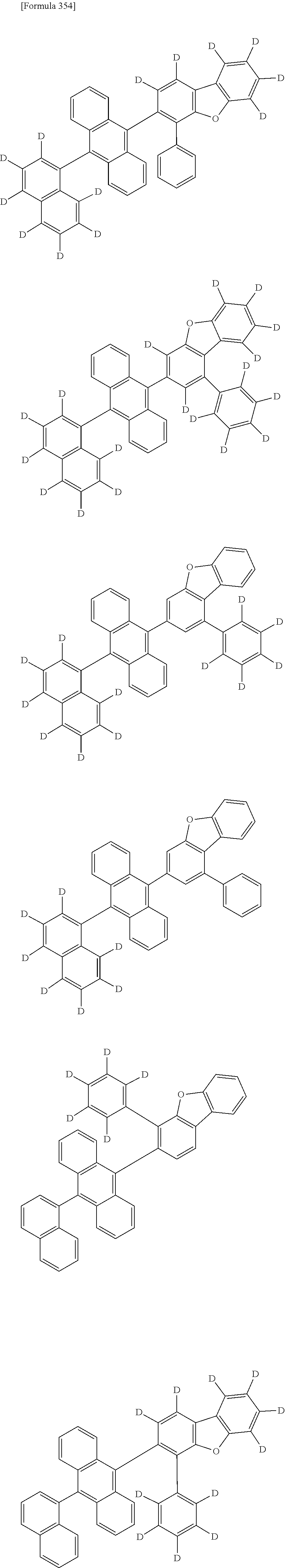

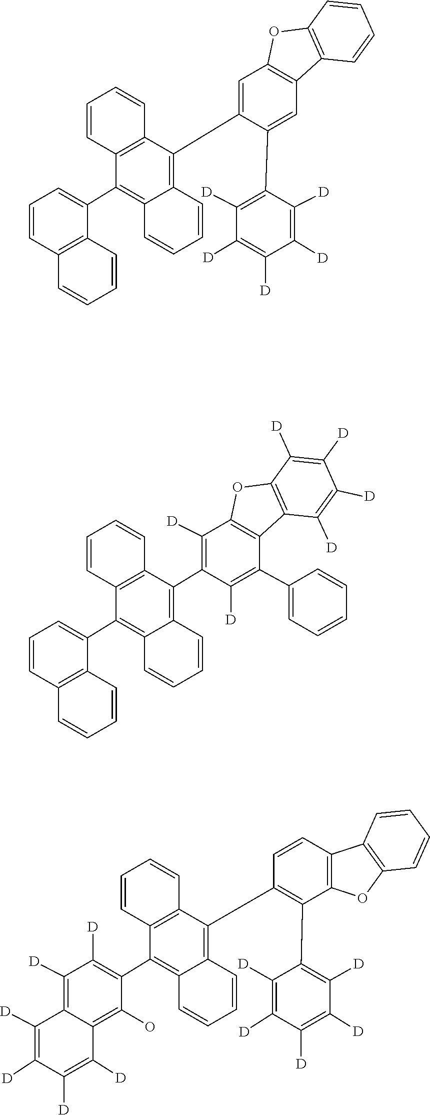

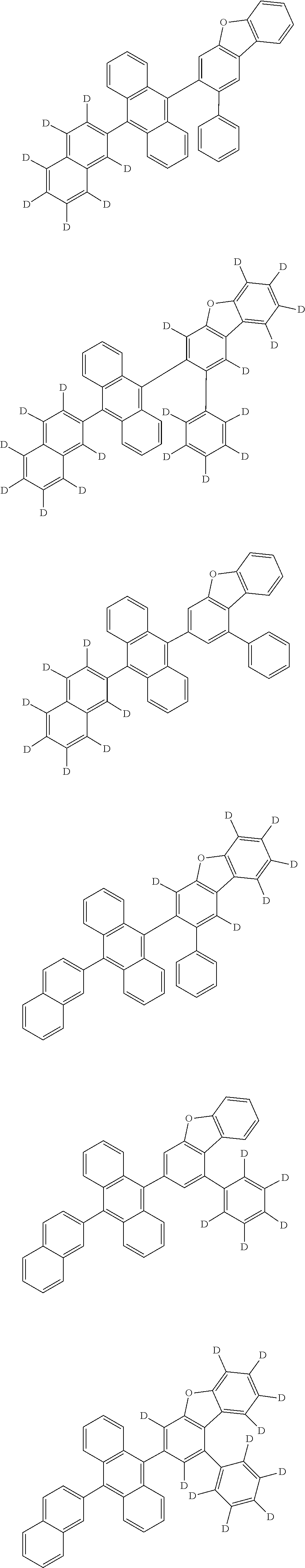

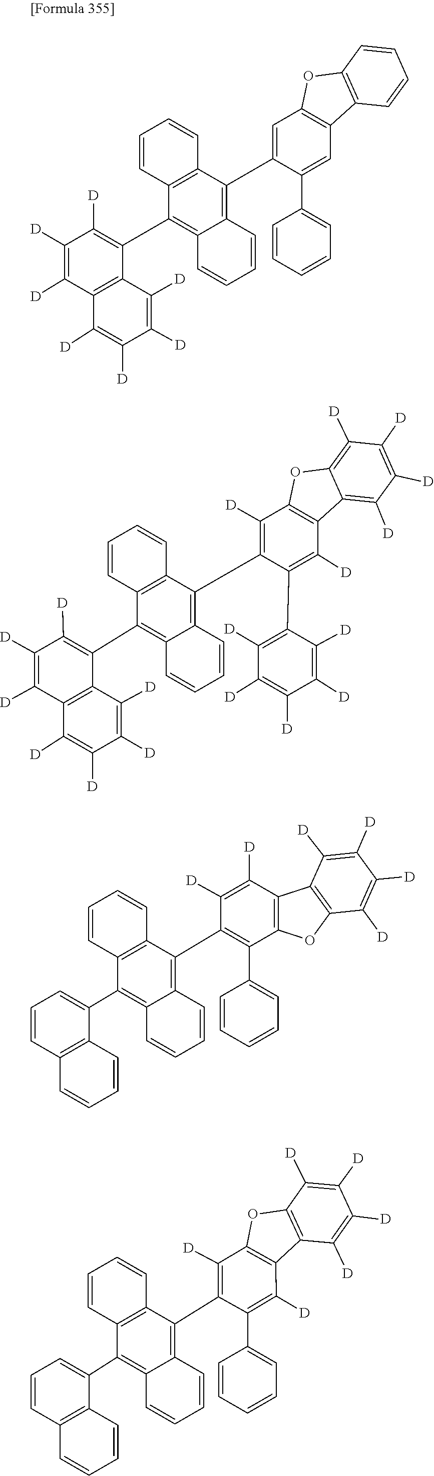 [SMILES: [2H]c1c([2H])c([2H])c2c(-c3c4ccccc4c(-c4cc5oc6ccccc6c5cc4-c4ccccc4)c4ccccc34)c([2H])c([2H])c([2H])c2c1[2H] ; [2H]c1c([2H])c([2H])c(-c2c(-c3c4ccccc4c(-c4c([2H])c([2H])c([2H])c5c([2H])c([2H])c([2H])c([2H])c45)c4ccccc34)c([2H])c3oc4c([2H])c([2H])c([2H])c([2H])c4c3c2[2H])c([2H])c1[2H] ; [2H]c1c([2H])c([2H])c2c(oc3c(-c4ccccc4)c(-c4c5ccccc5c(-c5cccc6ccccc56)c5ccccc45)c([2H])c([2H])c32)c1[2H] ; [2H]c1c([2H])c([2H])c2c(oc3c([2H])c(-c4c5ccccc5c(-c5cccc6ccccc56)c5ccccc45)c(-c4ccccc4)c([2H])c32)c1[2H]]

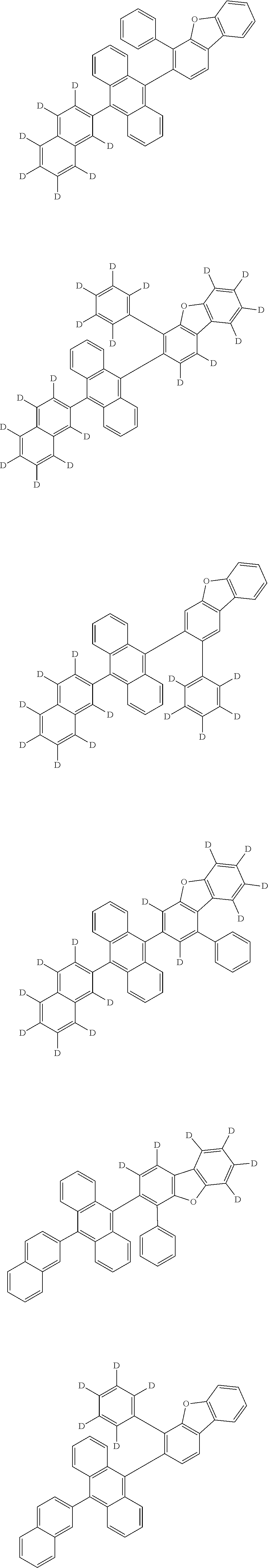

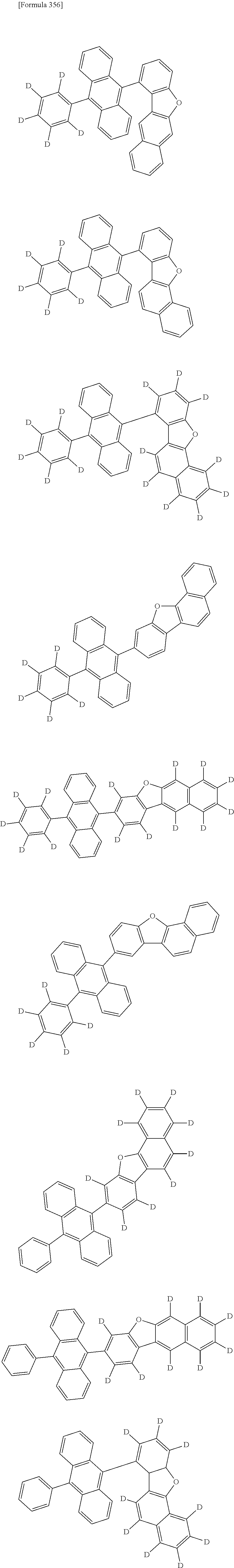

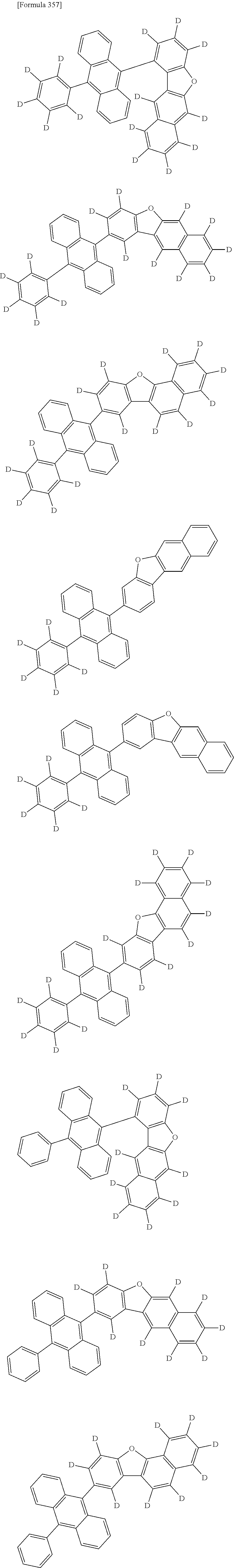

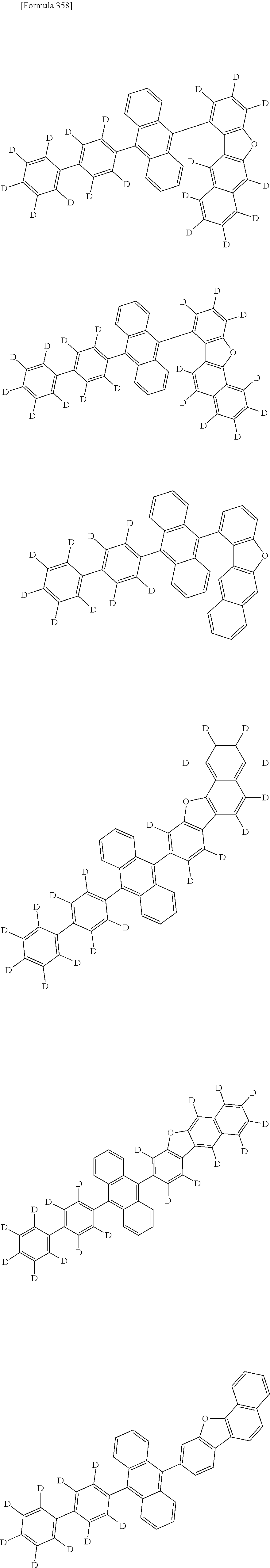

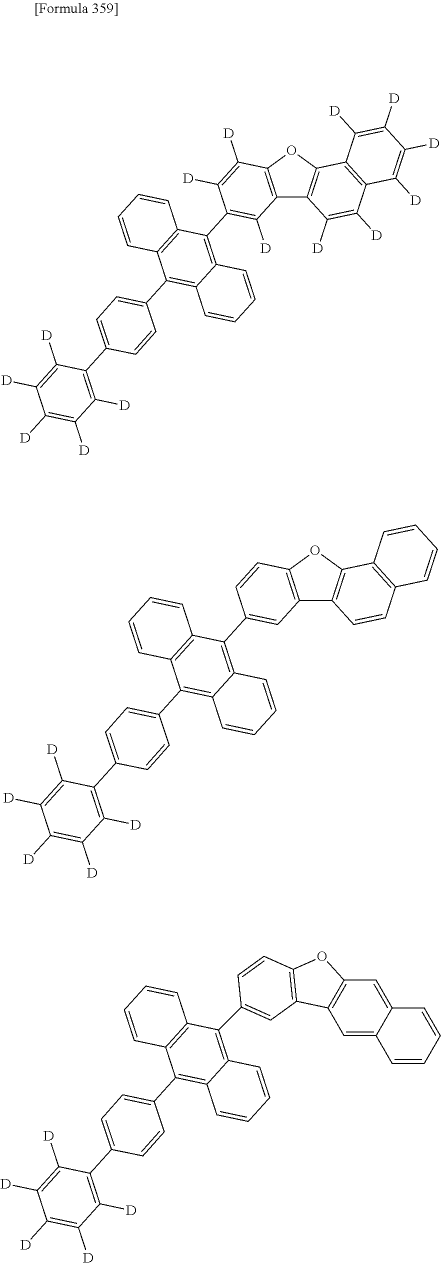 [SMILES: [2H]c1c([2H])c([2H])c(-c2ccc(-c3c4ccccc4c(-c4c([2H])c([2H])c5oc6c7c([2H])c([2H])c([2H])c([2H])c7c([2H])c([2H])c6c5c4[2H])c4ccccc34)cc2)c([2H])c1[2H] ; [2H]c1c([2H])c([2H])c(-c2ccc(-c3c4ccccc4c(-c4ccc5oc6c7ccccc7ccc6c5c4)c4ccccc34)cc2)c([2H])c1[2H] ; [2H]c1c([2H])c([2H])c(-c2ccc(-c3c4ccccc4c(-c4ccc5oc6cc7ccccc7cc6c5c4)c4ccccc34)cc2)c([2H])c1[2H]]

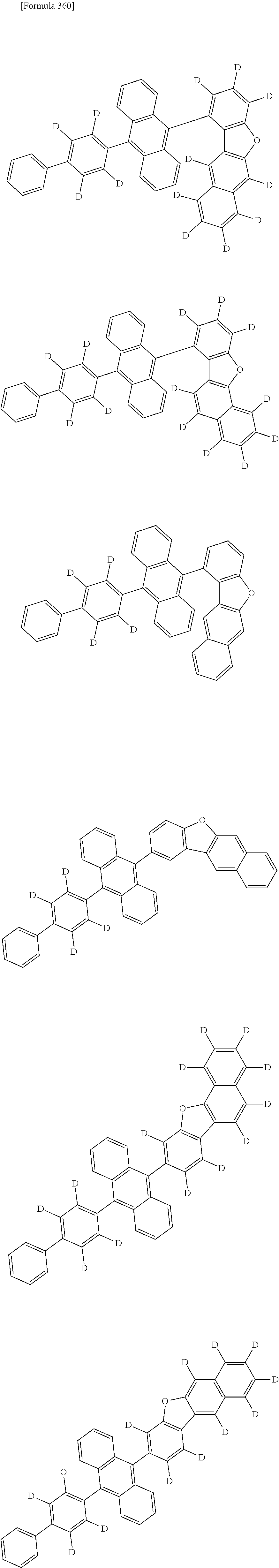

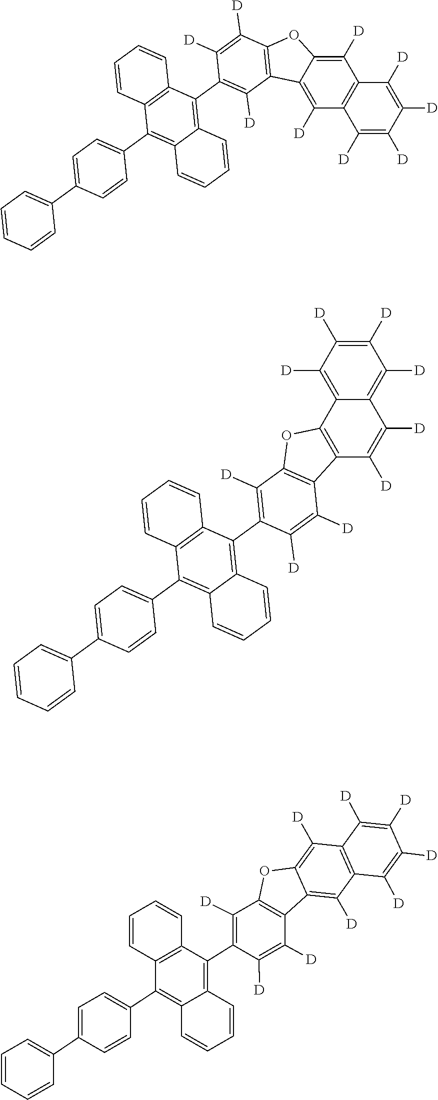 [SMILES: [2H]c1c(-c2c3ccccc3c(-c3ccc(-c4ccccc4)cc3)c3ccccc23)c([2H])c2c(oc3c([2H])c4c([2H])c([2H])c([2H])c([2H])c4c([2H])c32)c1[2H] ; [2H]c1c([2H])c([2H])c2c(c1[2H])c([2H])c([2H])c1c2oc2c([2H])c(-c3c4ccccc4c(-c4ccc(-c5ccccc5)cc4)c4ccccc34)c([2H])c([2H])c21 ; [2H]c1c([2H])c([2H])c2c([2H])c3c(oc4c([2H])c(-c5c6ccccc6c(-c6ccc(-c7ccccc7)cc6)c6ccccc56)c([2H])c([2H])c43)c([2H])c2c1[2H]]

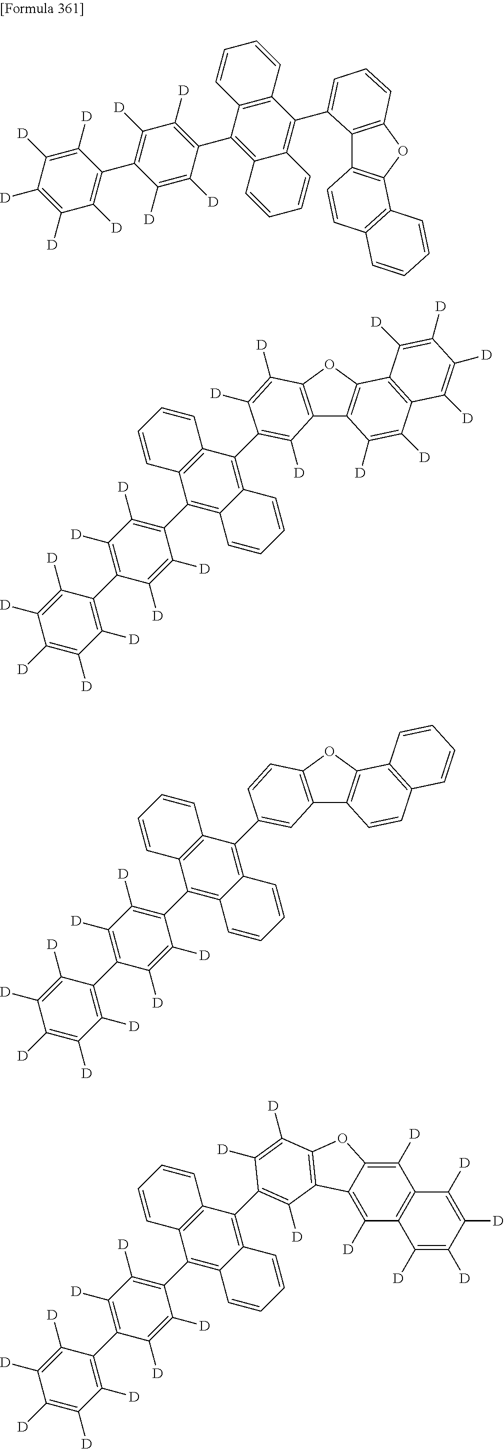

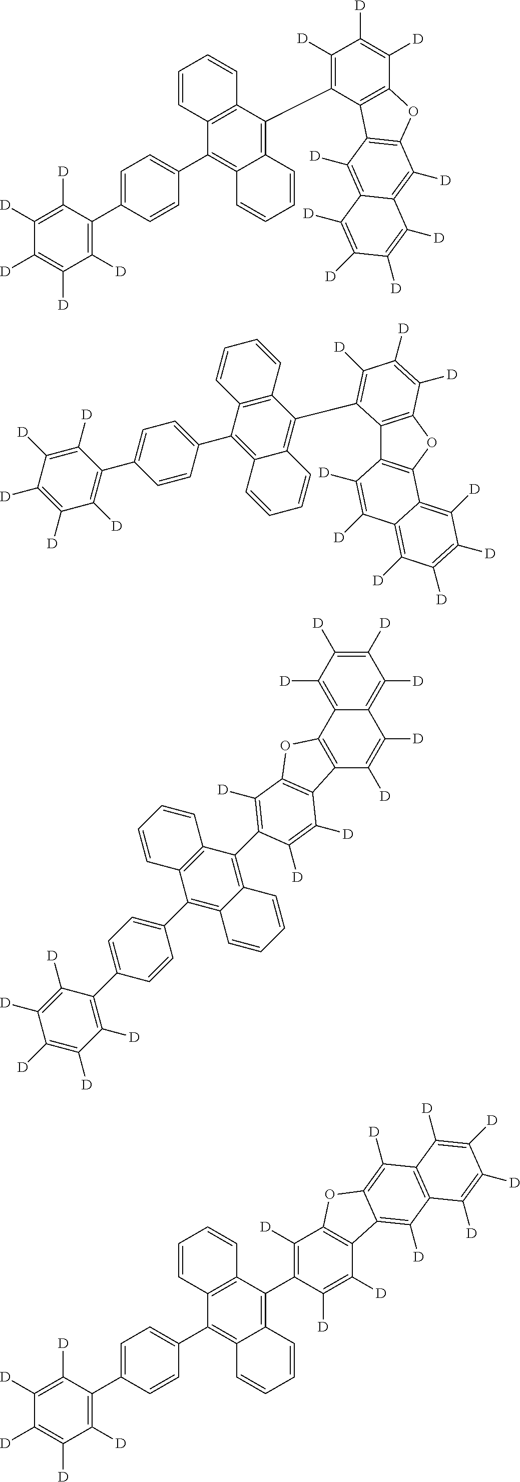

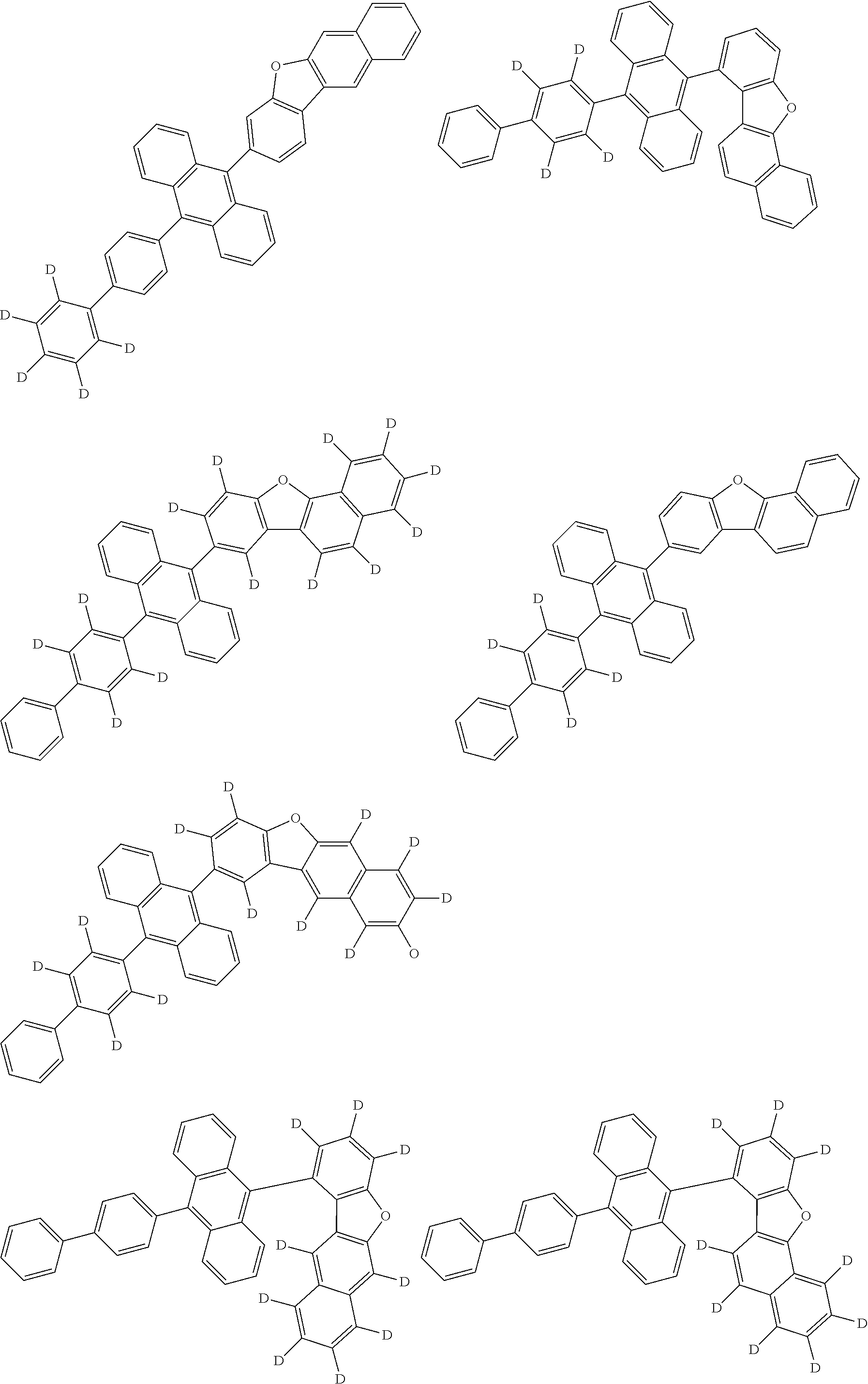

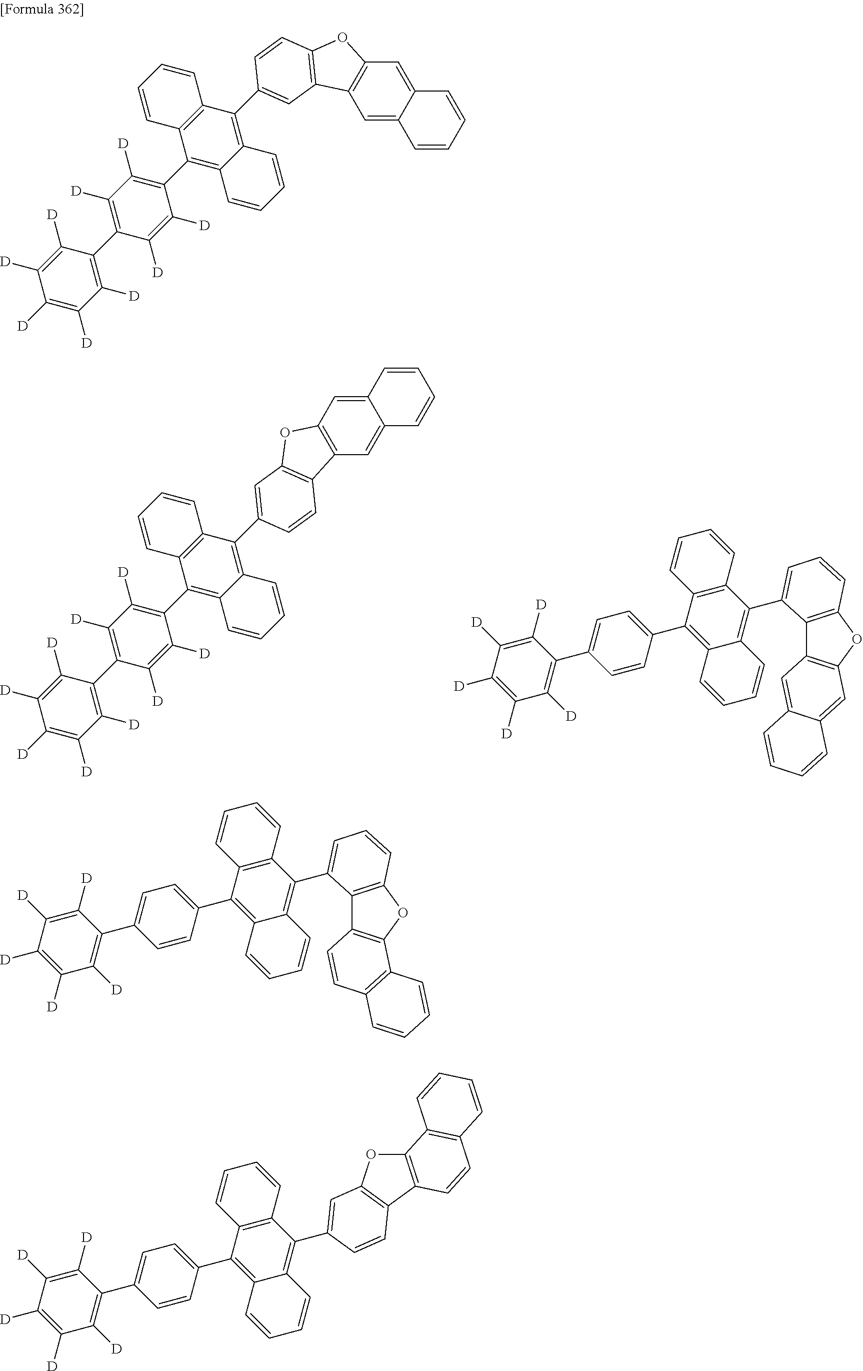

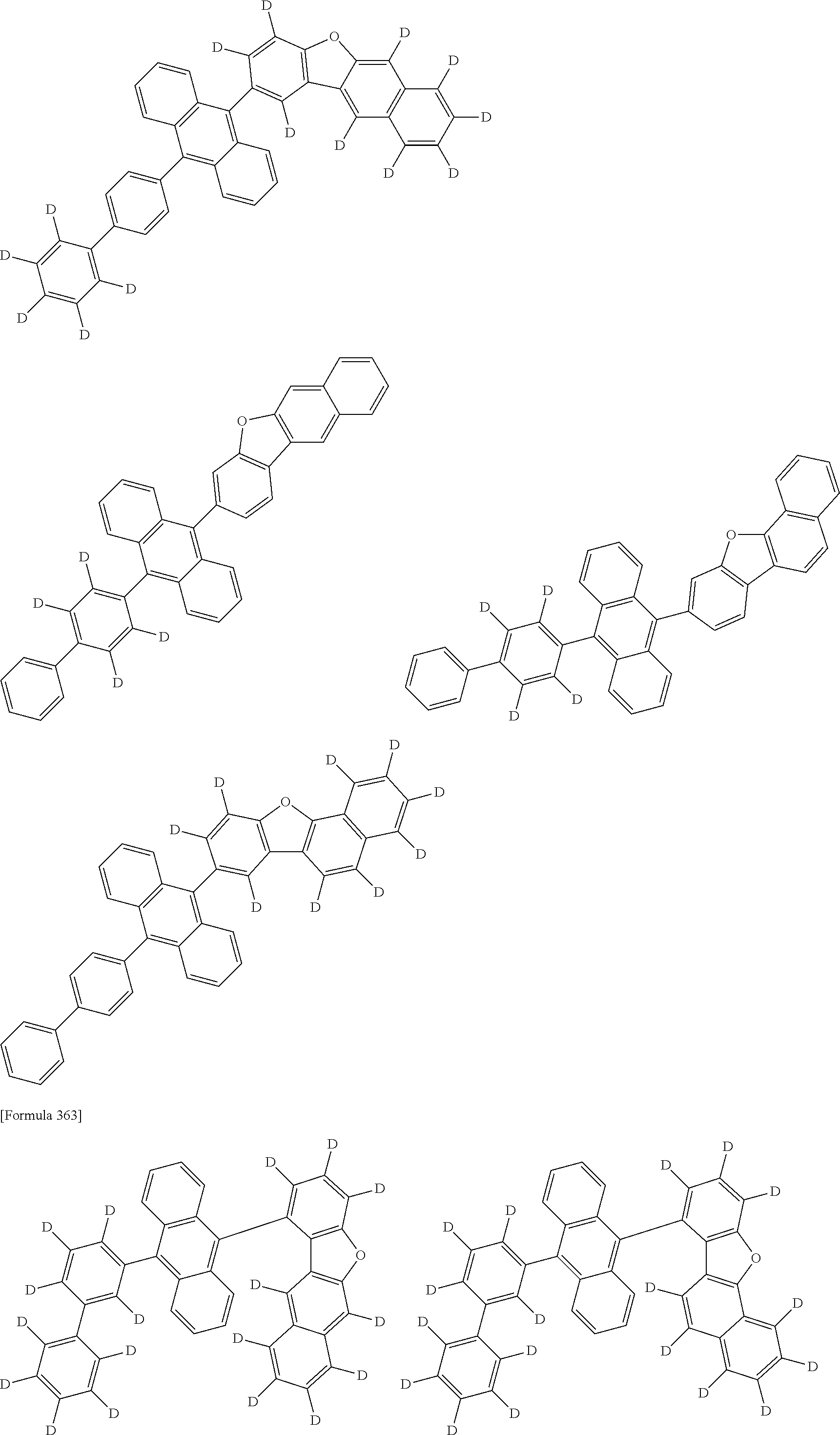

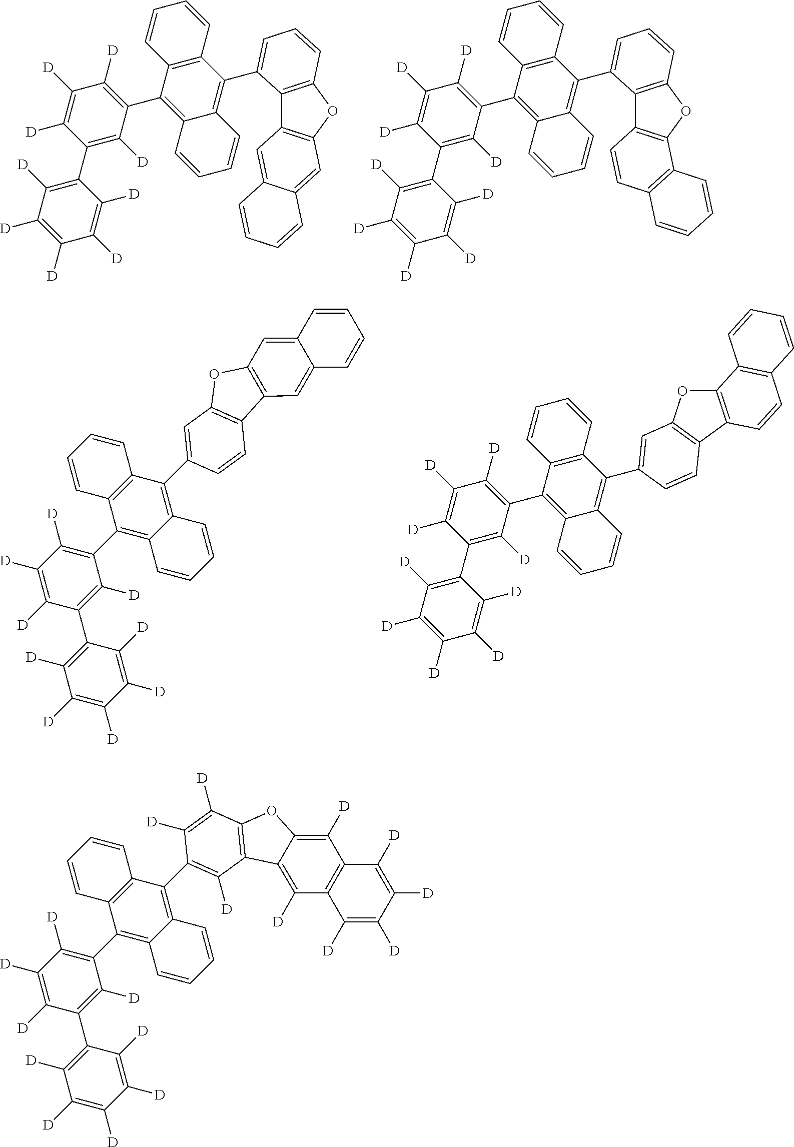

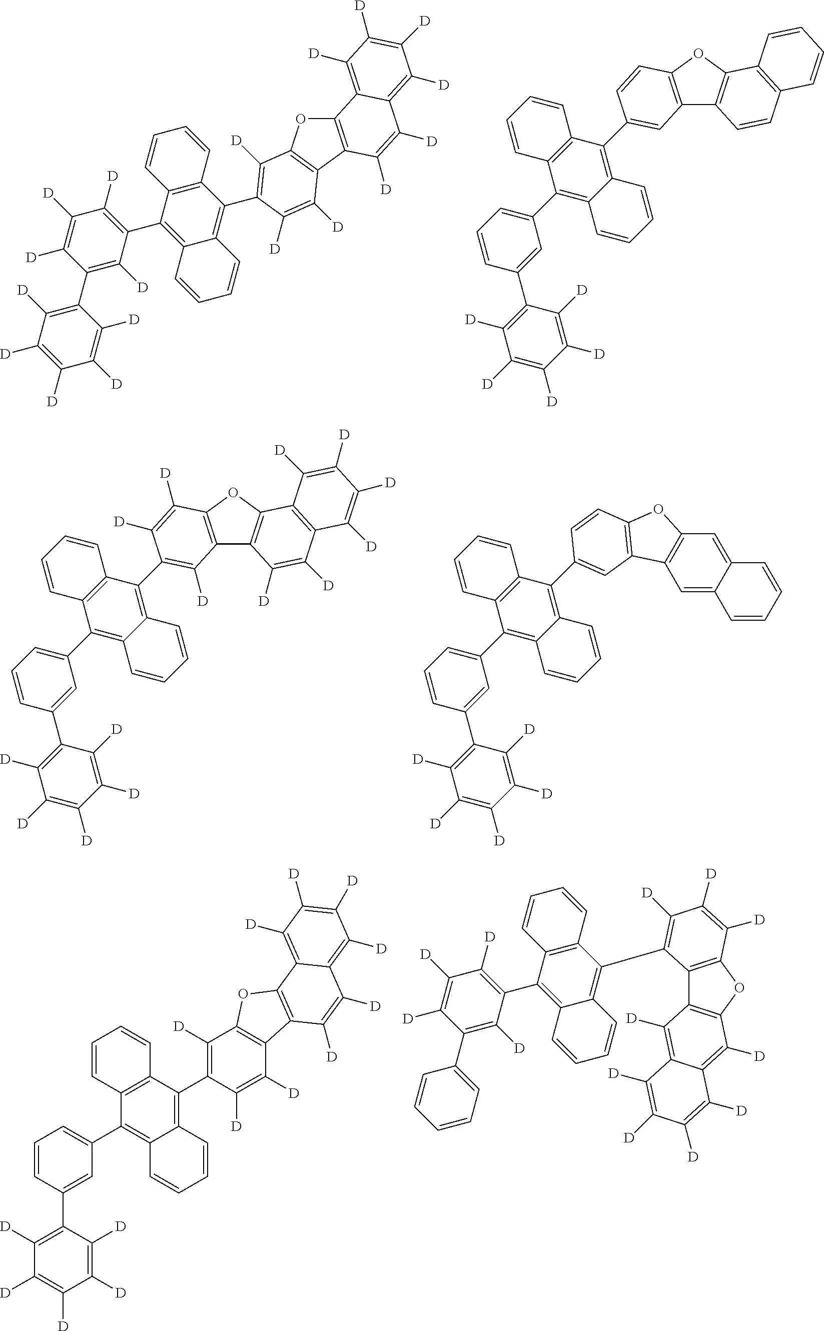

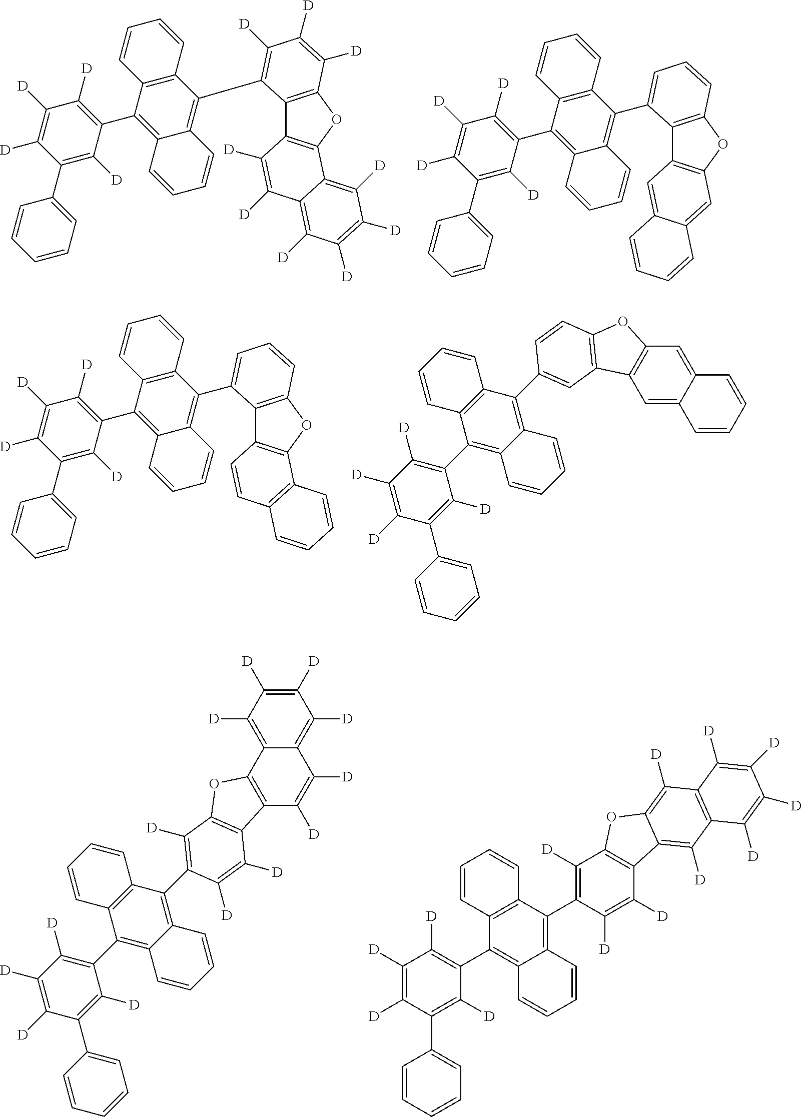

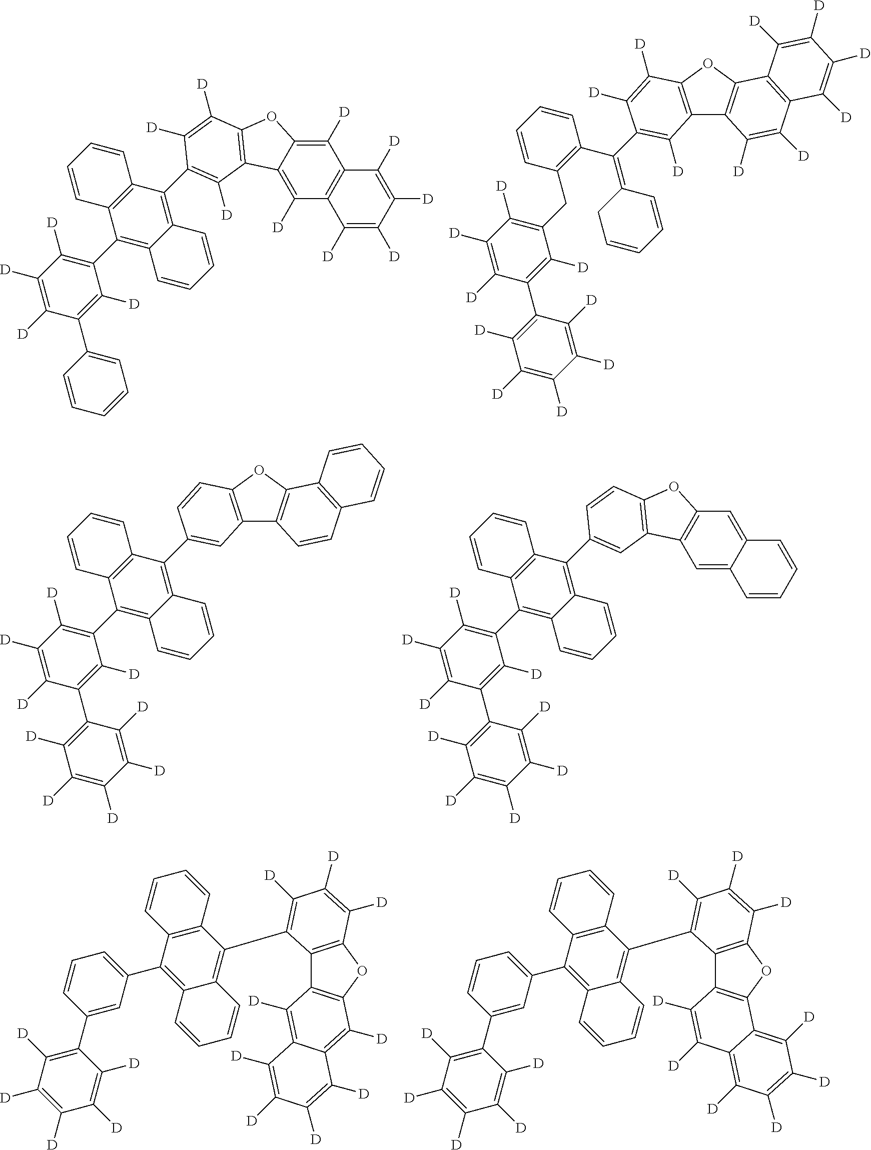

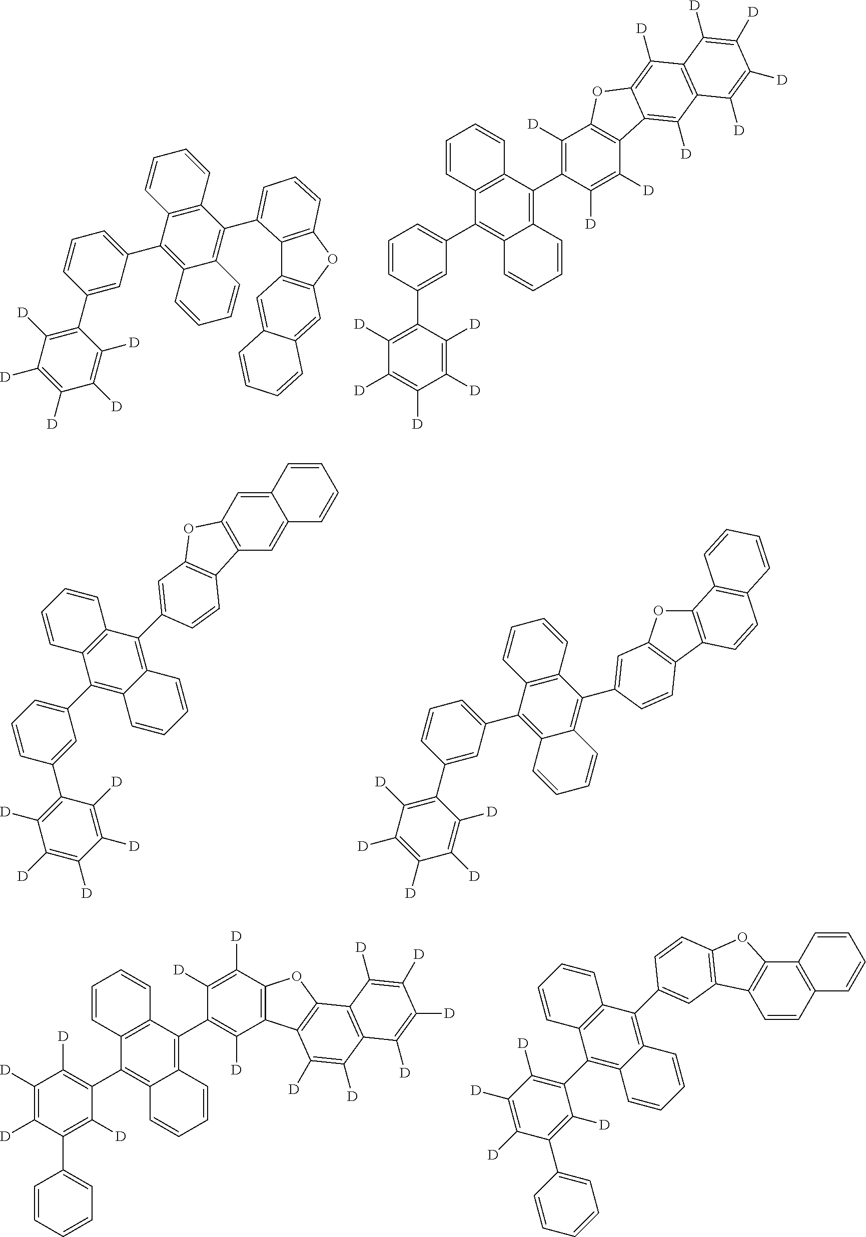

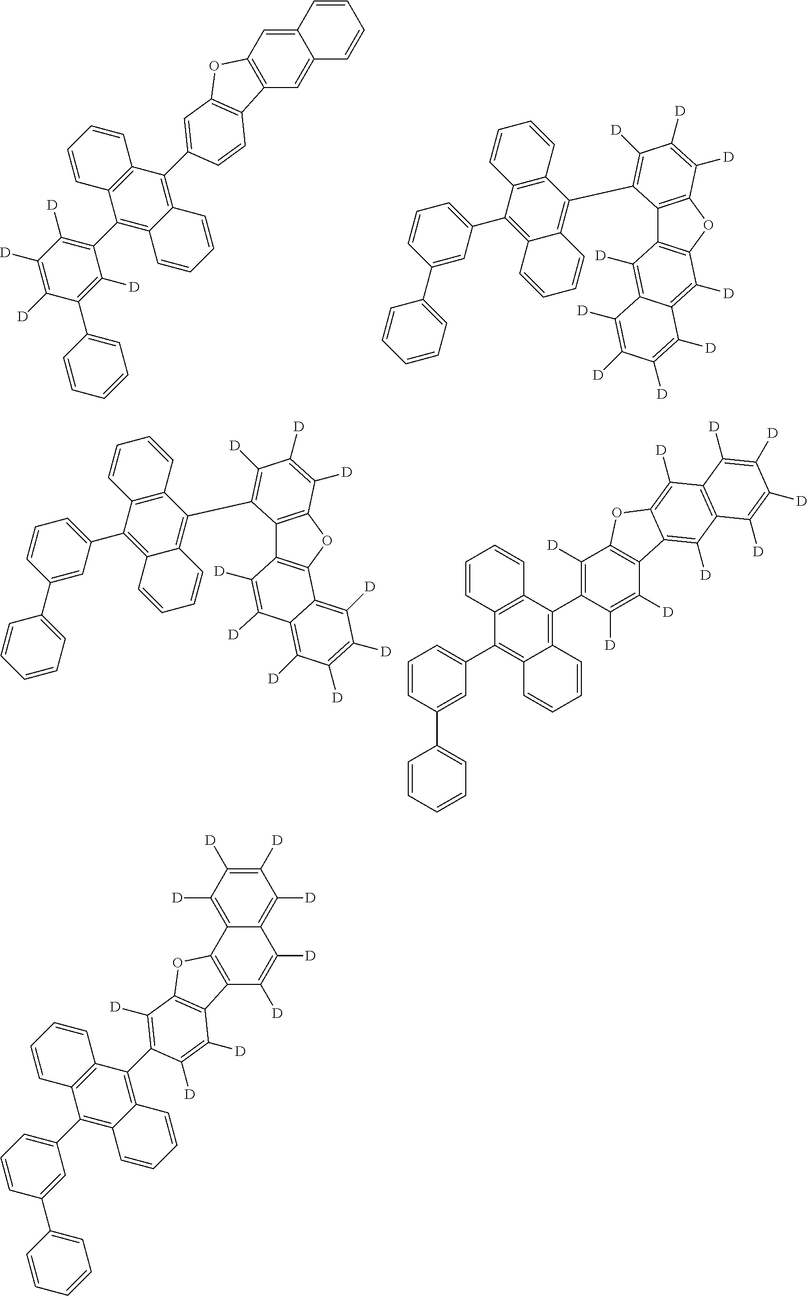

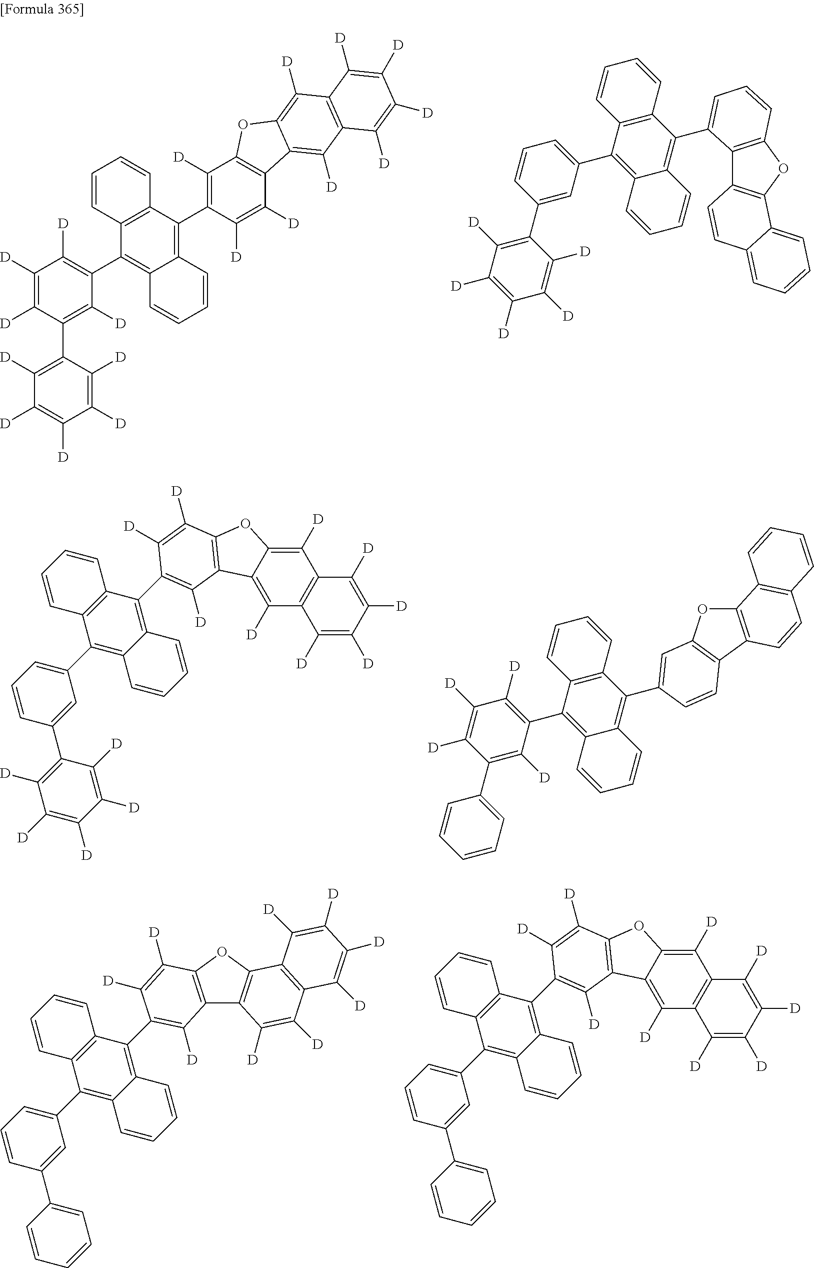

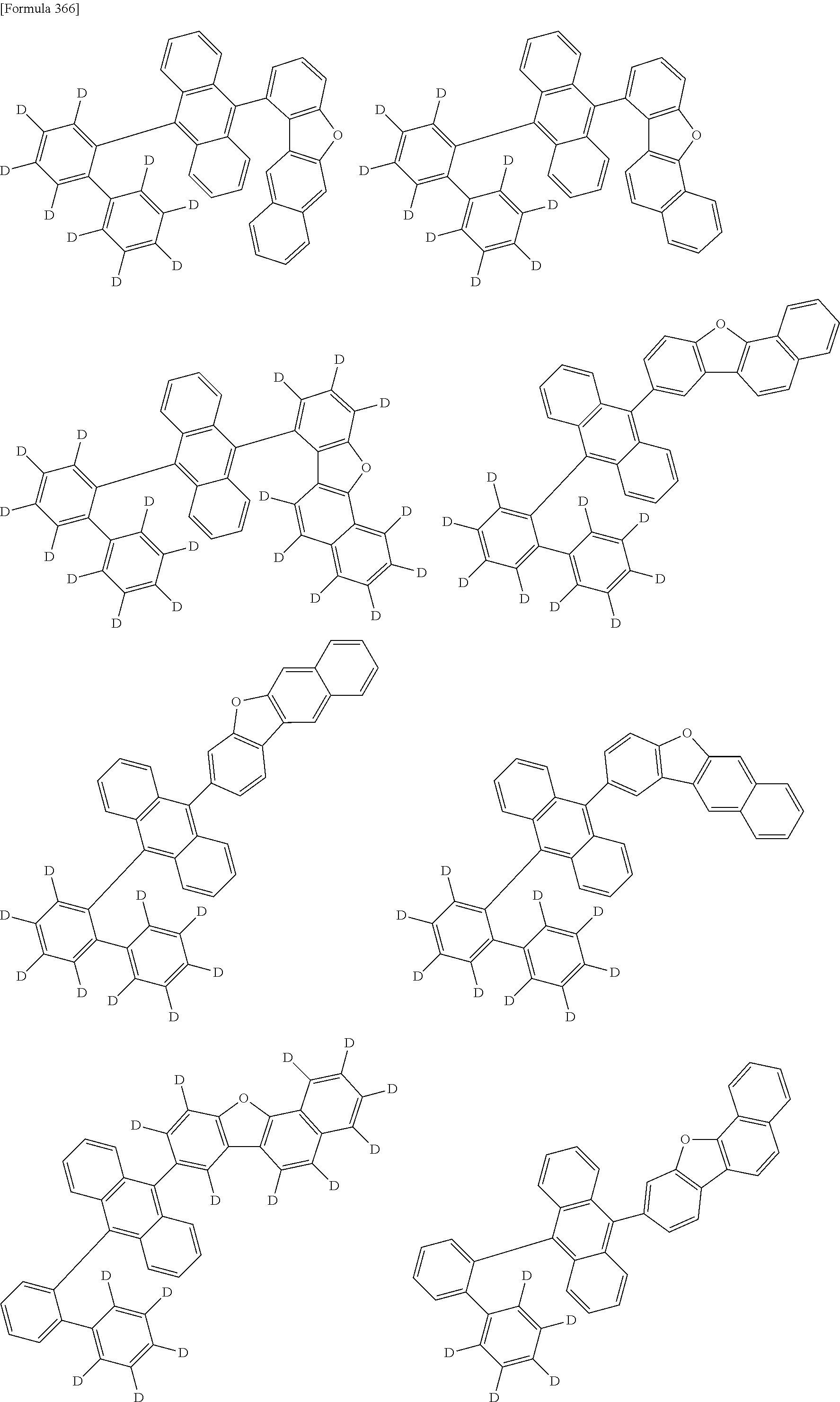

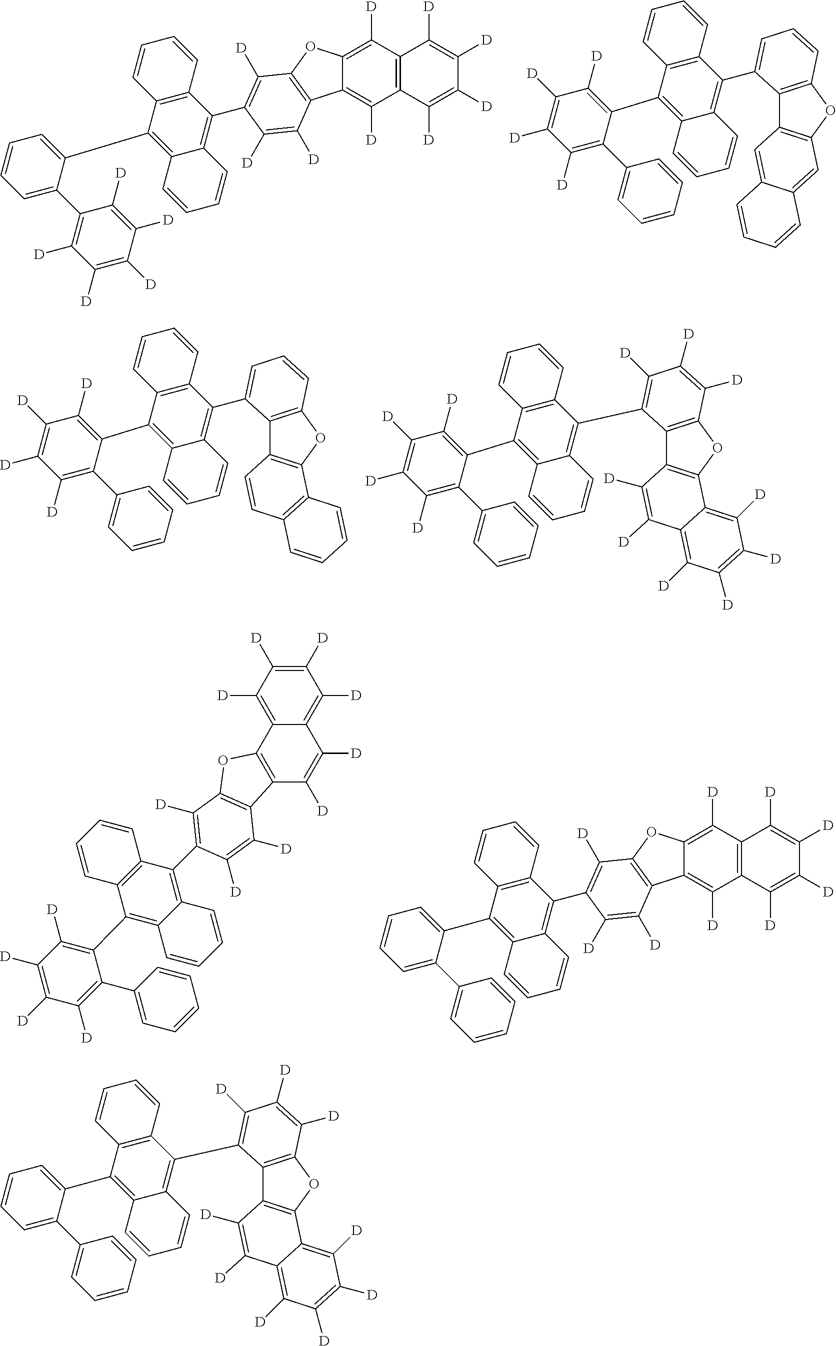

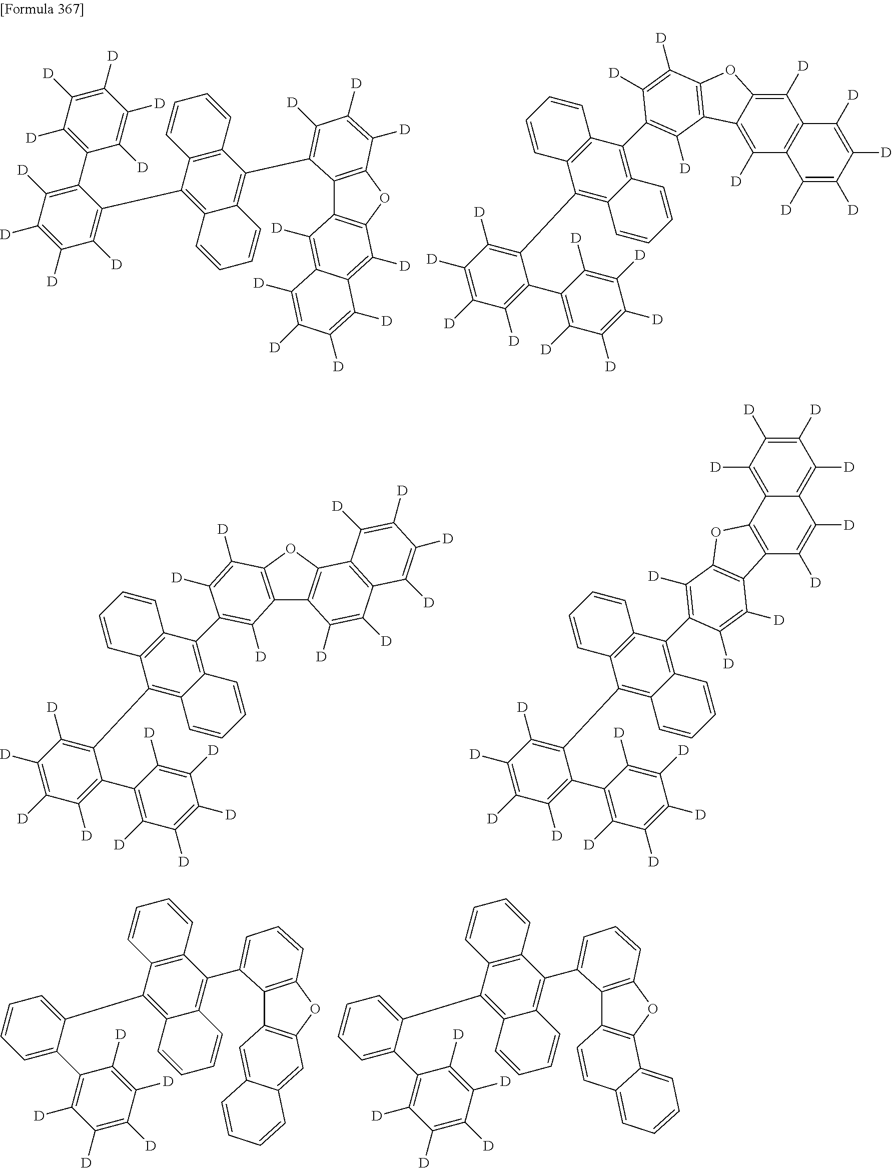

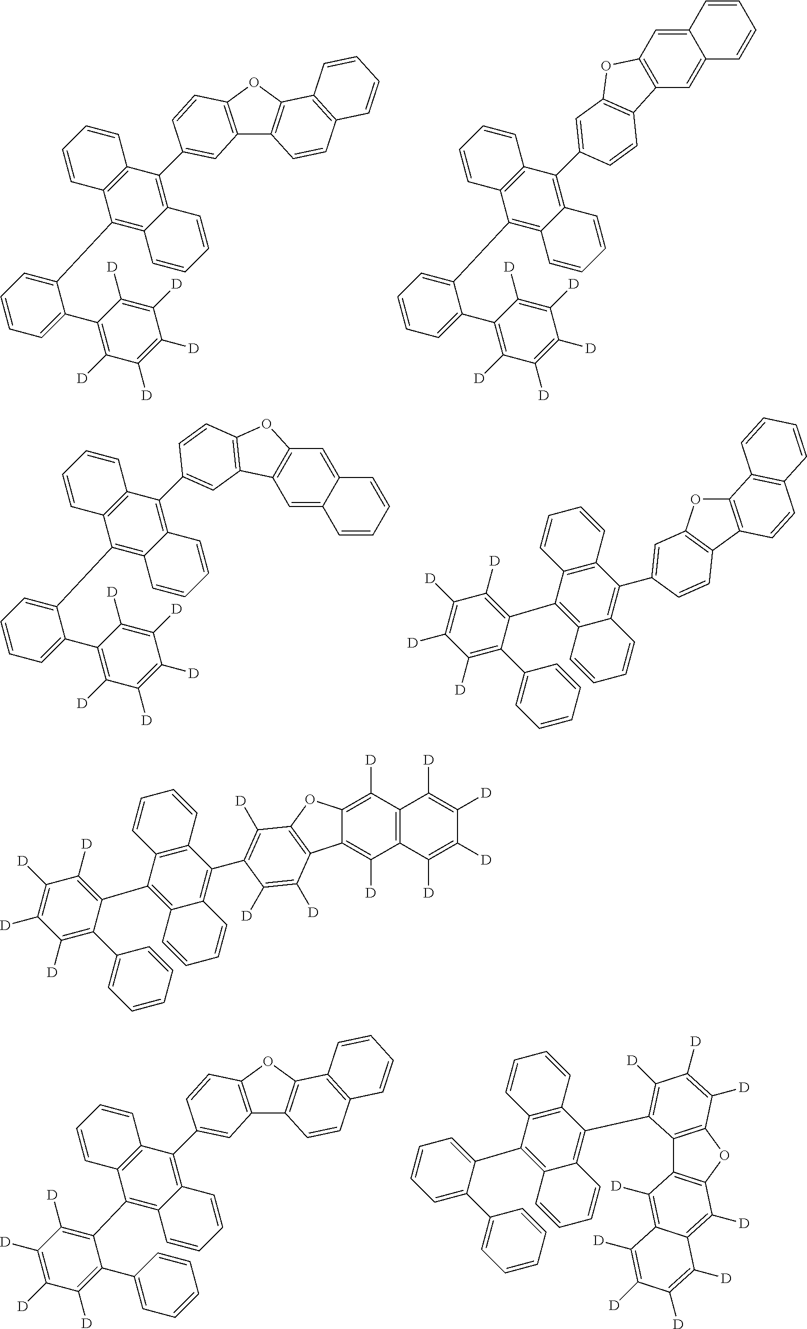

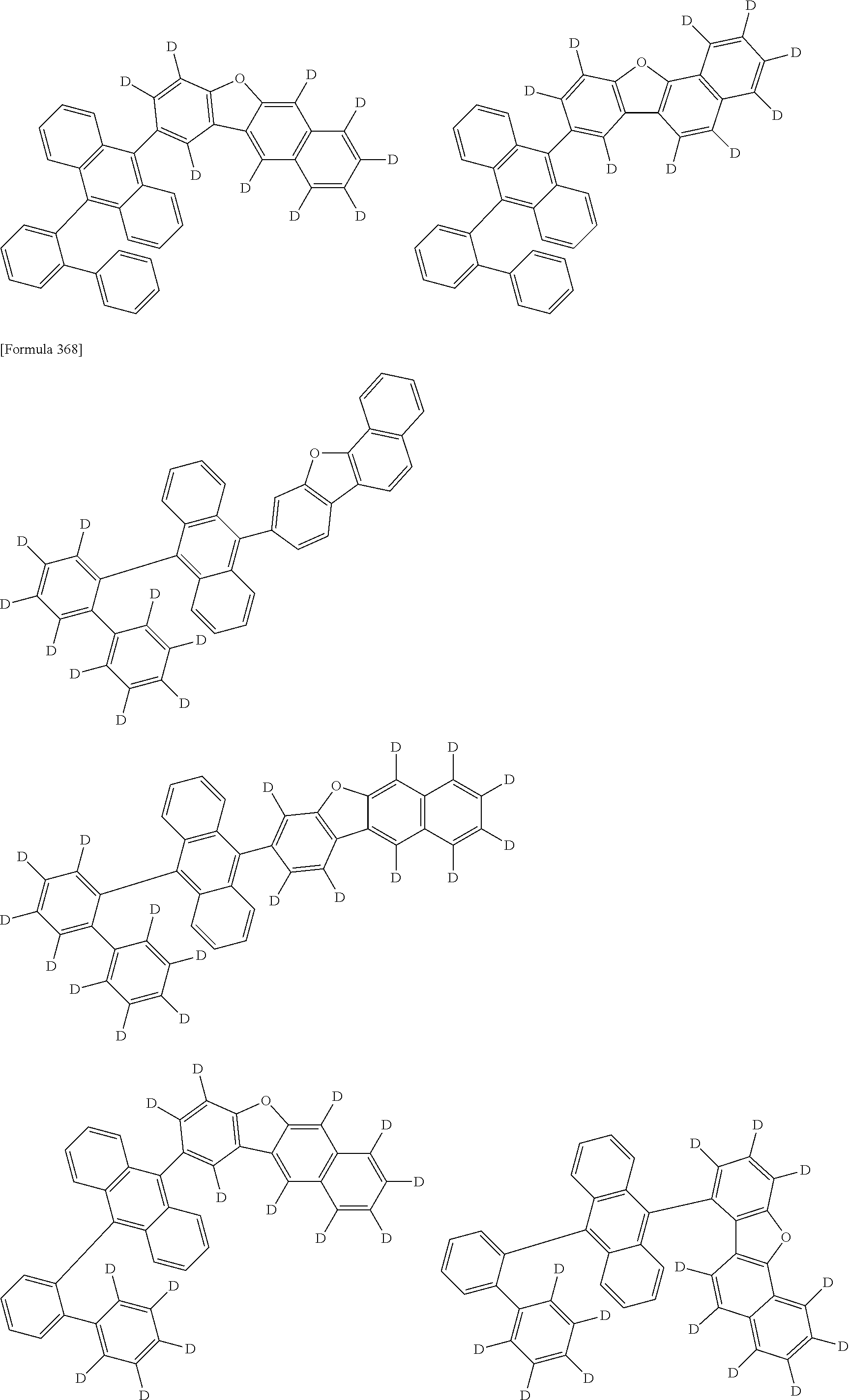

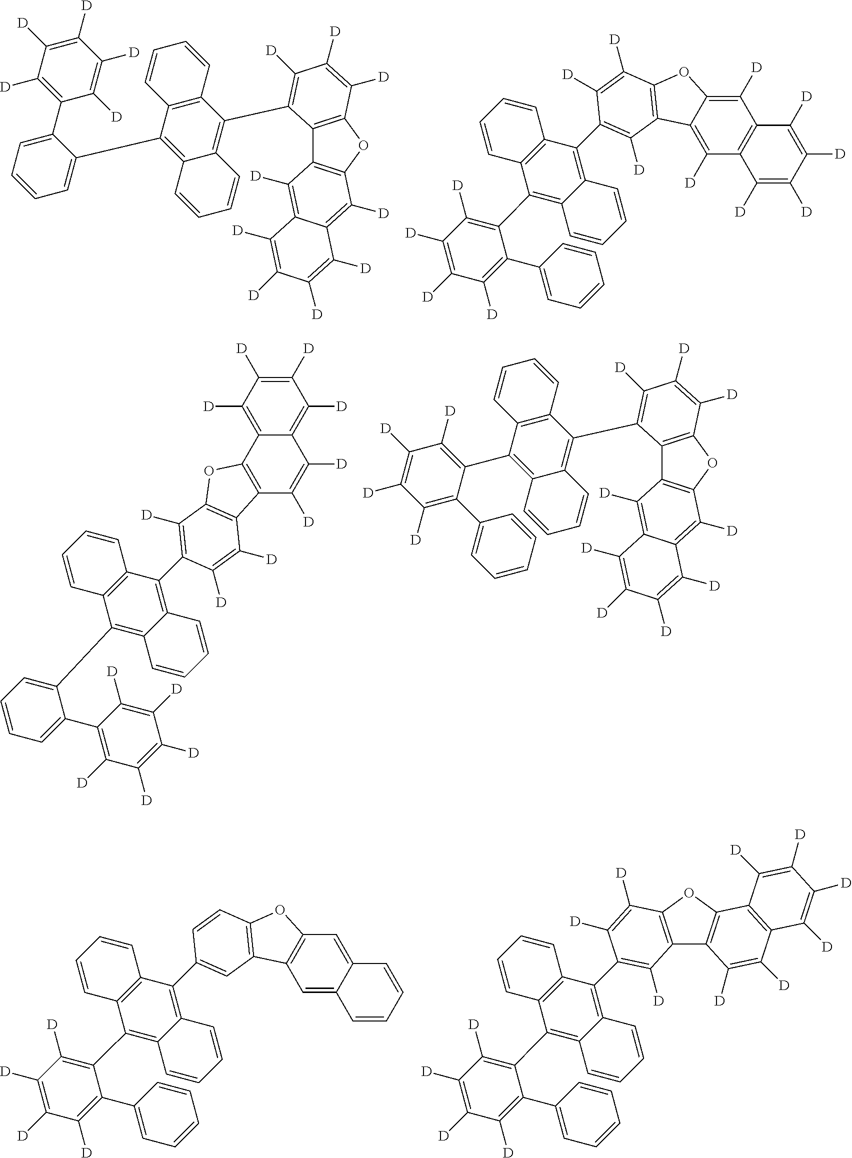

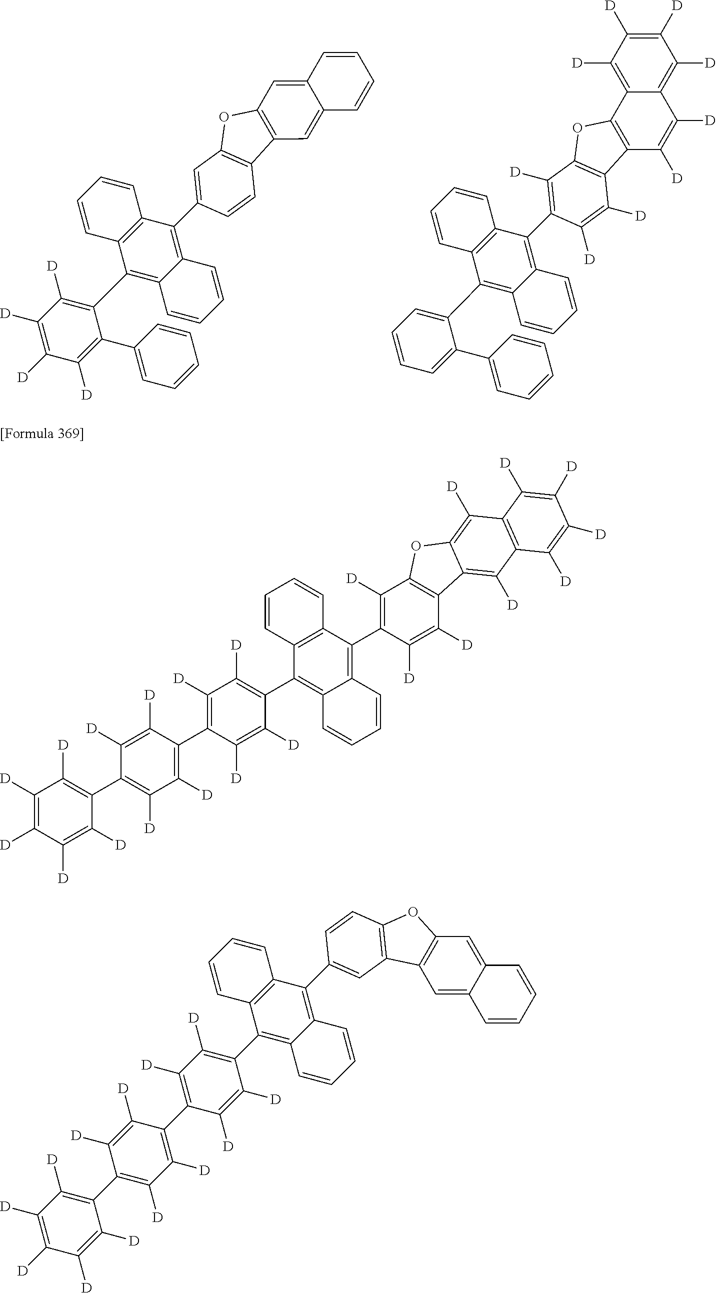

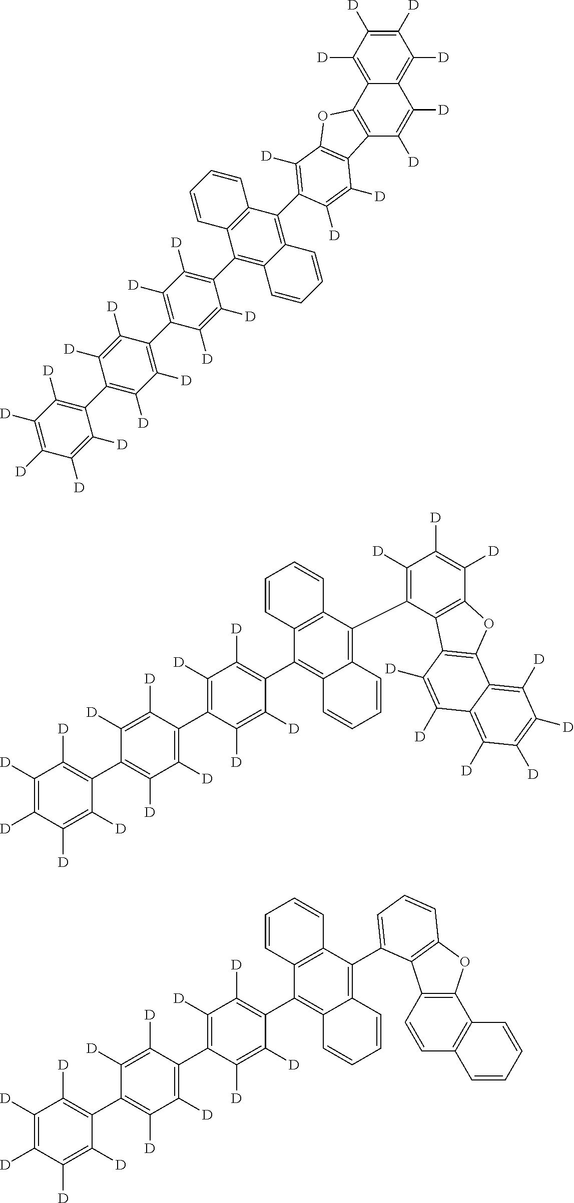

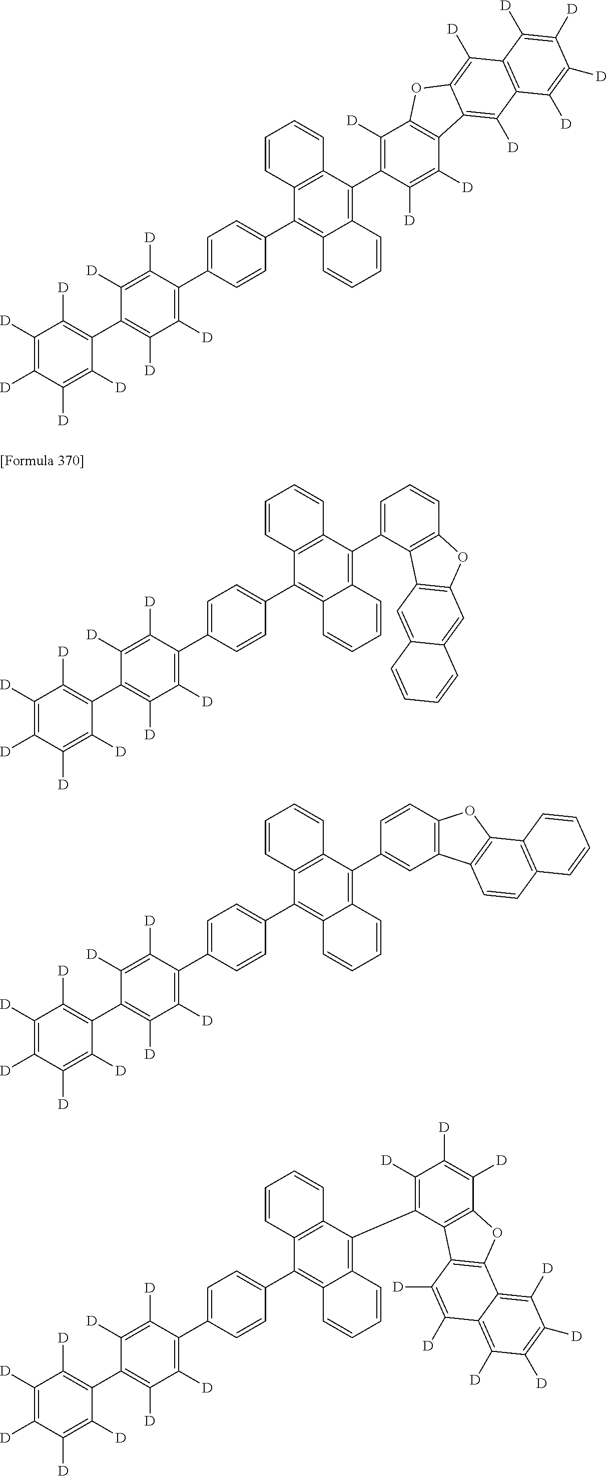

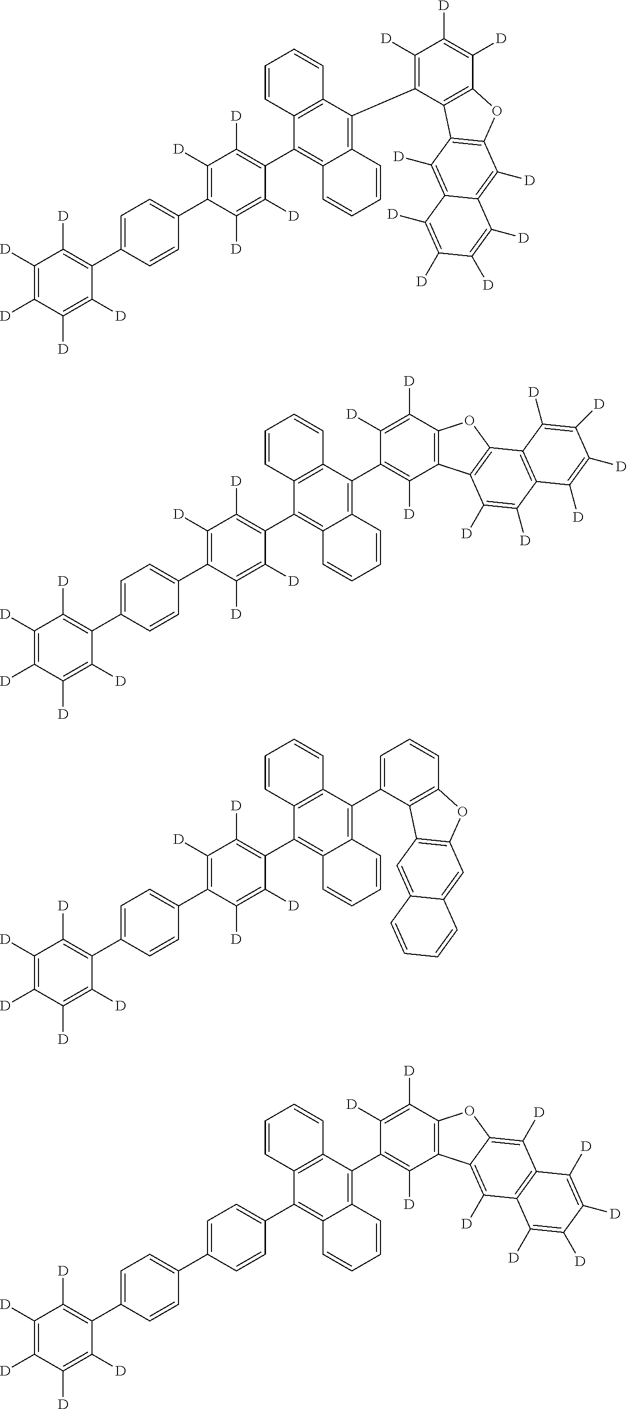

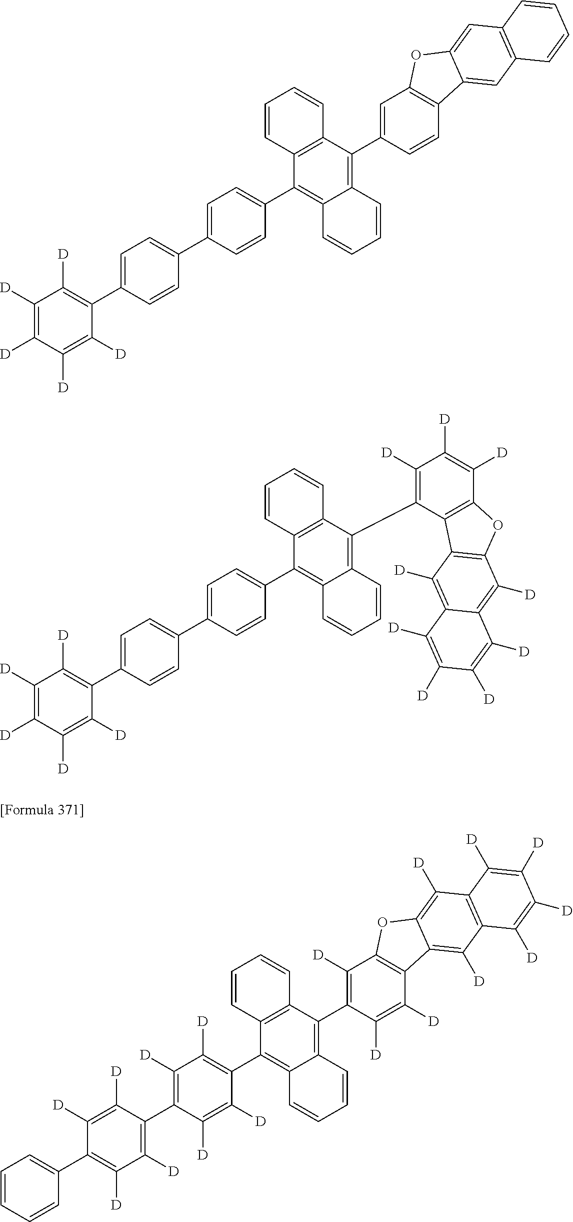 [SMILES: [2H]c1c([2H])c([2H])c(-c2ccc(-c3ccc(-c4c5ccccc5c(-c5ccc6c(c5)oc5cc7ccccc7cc56)c5ccccc45)cc3)cc2)c([2H])c1[2H] ; [2H]c1c([2H])c([2H])c(-c2ccc(-c3ccc(-c4c5ccccc5c(-c5c([2H])c([2H])c([2H])c6oc7c([2H])c8c([2H])c([2H])c([2H])c([2H])c8c([2H])c7c56)c5ccccc45)cc3)cc2)c([2H])c1[2H] ; [2H]c1c([2H])c(-c2c([2H])c([2H])c(-c3c4ccccc4c(-c4c([2H])c([2H])c5c(oc6c([2H])c7c([2H])c([2H])c([2H])c([2H])c7c([2H])c65)c4[2H])c4ccccc34)c([2H])c2[2H])c([2H])c([2H])c1-c1ccccc1]

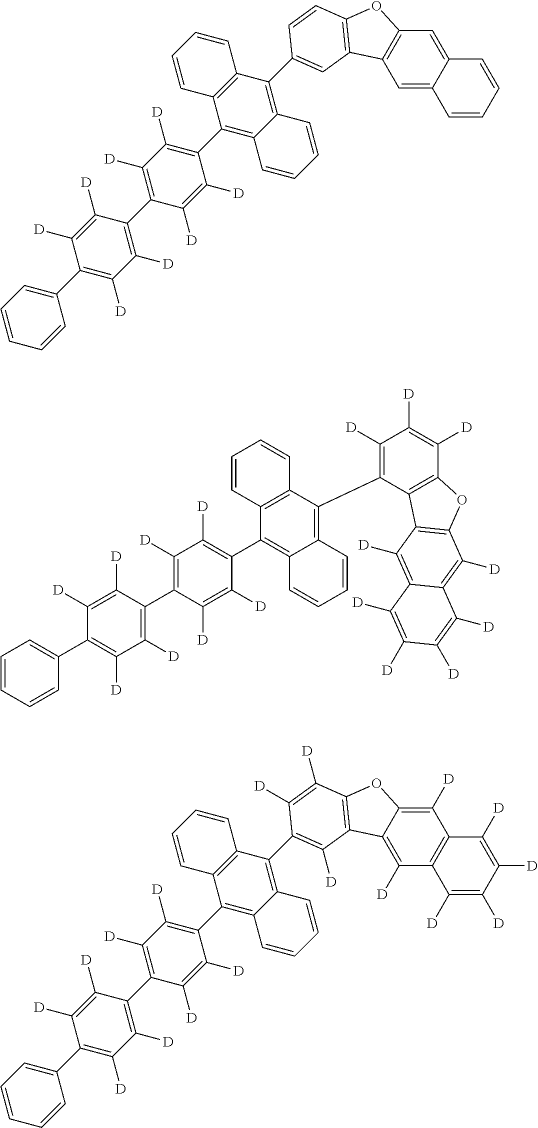 [SMILES: [2H]c1c([2H])c(-c2c([2H])c([2H])c(-c3c4ccccc4c(-c4ccc5oc6cc7ccccc7cc6c5c4)c4ccccc34)c([2H])c2[2H])c([2H])c([2H])c1-c1ccccc1 ; [2H]c1c([2H])c(-c2c([2H])c([2H])c(-c3c4ccccc4c(-c4c([2H])c([2H])c([2H])c5oc6c([2H])c7c([2H])c([2H])c([2H])c([2H])c7c([2H])c6c45)c4ccccc34)c([2H])c2[2H])c([2H])c([2H])c1-c1ccccc1 ; [2H]c1c([2H])c(-c2c([2H])c([2H])c(-c3c4ccccc4c(-c4c([2H])c([2H])c5oc6c([2H])c7c([2H])c([2H])c([2H])c([2H])c7c([2H])c6c5c4[2H])c4ccccc34)c([2H])c2[2H])c([2H])c([2H])c1-c1ccccc1]

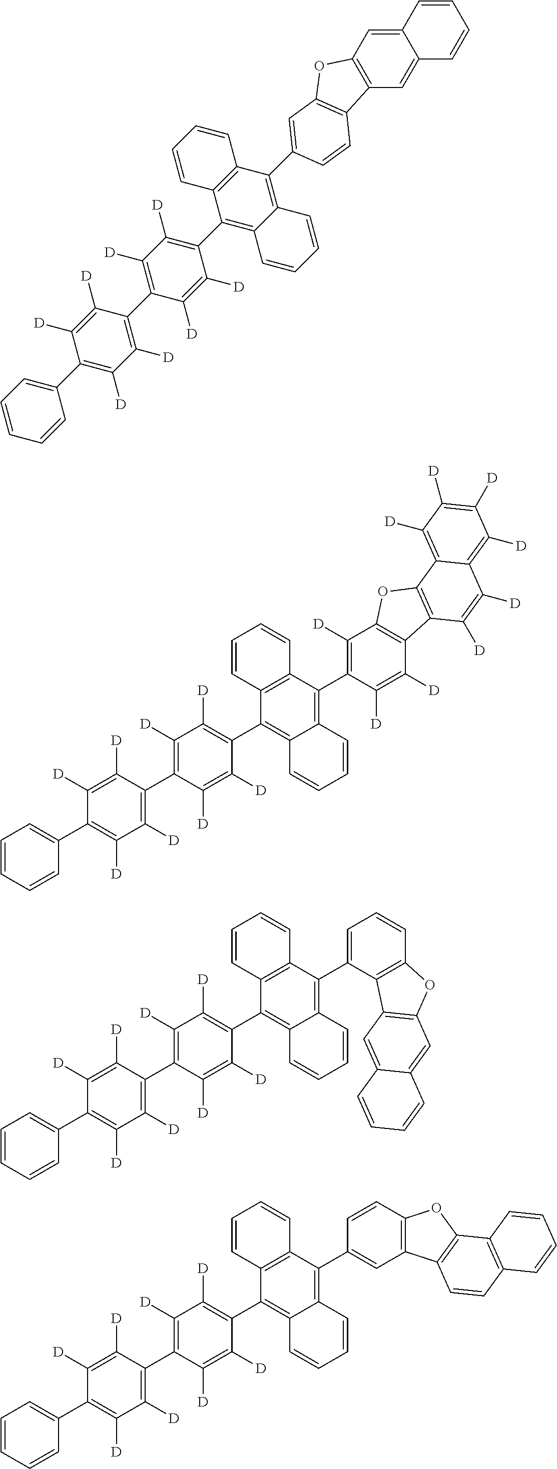

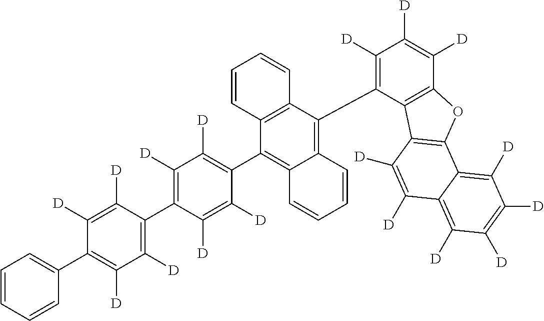 [SMILES: [2H]c1c([2H])c(-c2c([2H])c([2H])c(-c3c4ccccc4c(-c4c([2H])c([2H])c([2H])c5oc6c7c([2H])c([2H])c([2H])c([2H])c7c([2H])c([2H])c6c45)c4ccccc34)c([2H])c2[2H])c([2H])c([2H])c1-c1ccccc1]

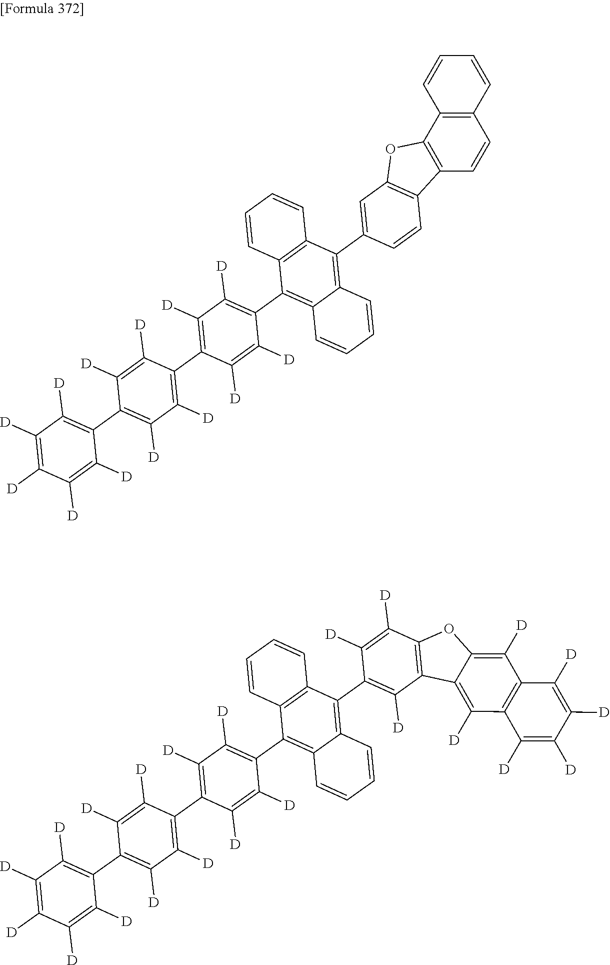 [SMILES: [2H]c1c([2H])c([2H])c(-c2c([2H])c([2H])c(-c3c([2H])c([2H])c(-c4c5ccccc5c(-c5ccc6c(c5)oc5c7ccccc7ccc65)c5ccccc45)c([2H])c3[2H])c([2H])c2[2H])c([2H])c1[2H] ; [2H]c1c([2H])c([2H])c(-c2c([2H])c([2H])c(-c3c([2H])c([2H])c(-c4c5ccccc5c(-c5c([2H])c([2H])c6oc7c([2H])c8c([2H])c([2H])c([2H])c([2H])c8c([2H])c7c6c5[2H])c5ccccc45)c([2H])c3[2H])c([2H])c2[2H])c([2H])c1[2H]]

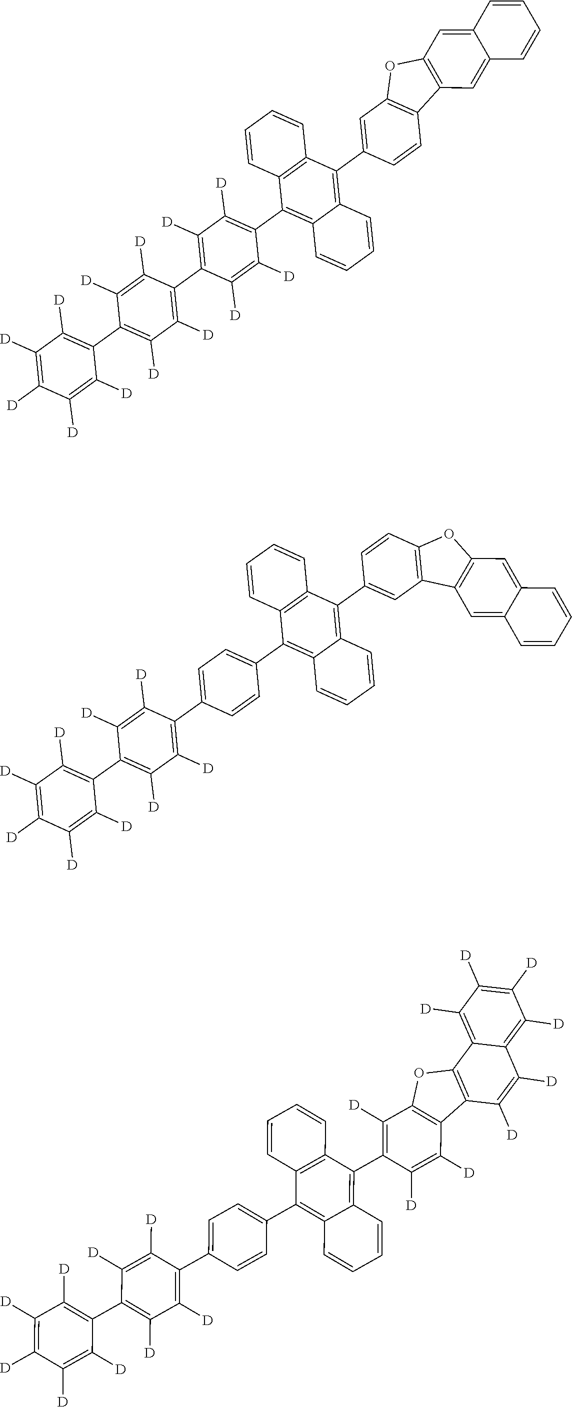 [SMILES: [2H]c1c([2H])c([2H])c(-c2c([2H])c([2H])c(-c3c([2H])c([2H])c(-c4c5ccccc5c(-c5ccc6c(c5)oc5cc7ccccc7cc56)c5ccccc45)c([2H])c3[2H])c([2H])c2[2H])c([2H])c1[2H] ; [2H]c1c([2H])c([2H])c(-c2c([2H])c([2H])c(-c3ccc(-c4c5ccccc5c(-c5ccc6oc7cc8ccccc8cc7c6c5)c5ccccc45)cc3)c([2H])c2[2H])c([2H])c1[2H] ; [2H]c1c([2H])c([2H])c(-c2c([2H])c([2H])c(-c3ccc(-c4c5ccccc5c(-c5c([2H])c([2H])c6c(oc7c8c([2H])c([2H])c([2H])c([2H])c8c([2H])c([2H])c76)c5[2H])c5ccccc45)cc3)c([2H])c2[2H])c([2H])c1[2H]]

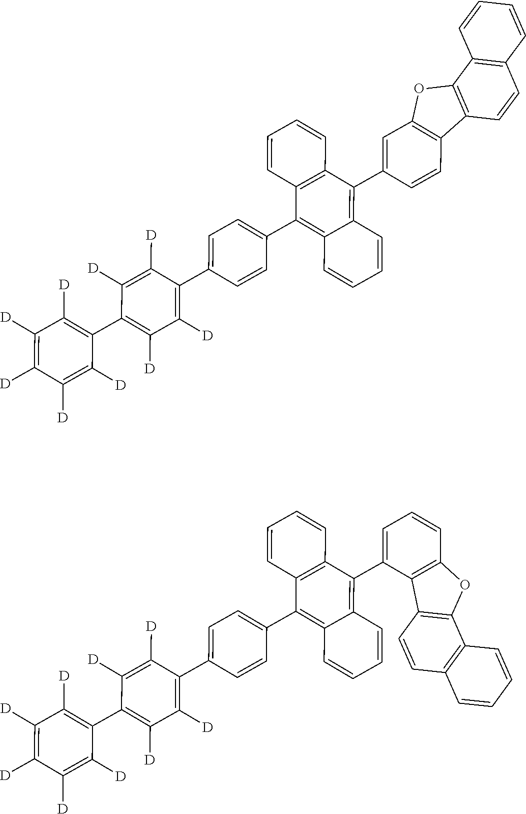 [SMILES: [2H]c1c([2H])c([2H])c(-c2c([2H])c([2H])c(-c3ccc(-c4c5ccccc5c(-c5ccc6c(c5)oc5c7ccccc7ccc65)c5ccccc45)cc3)c([2H])c2[2H])c([2H])c1[2H] ; [2H]c1c([2H])c([2H])c(-c2c([2H])c([2H])c(-c3ccc(-c4c5ccccc5c(-c5cccc6oc7c8ccccc8ccc7c56)c5ccccc45)cc3)c([2H])c2[2H])c([2H])c1[2H]]

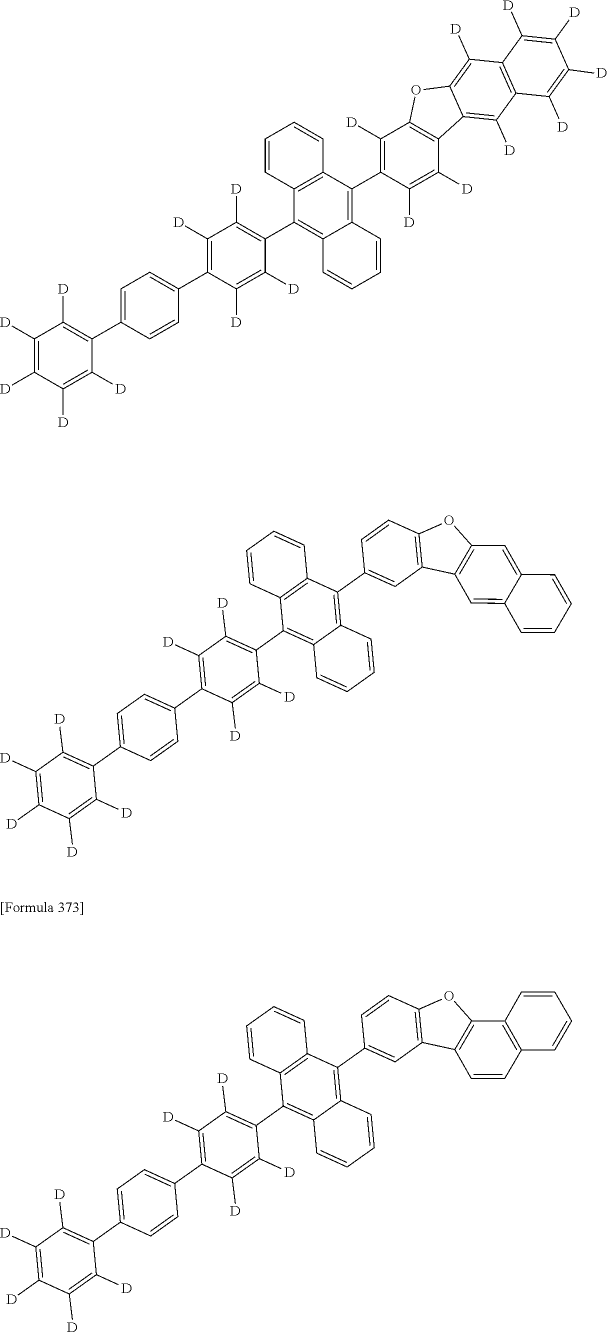 [SMILES: [2H]c1c([2H])c([2H])c(-c2ccc(-c3c([2H])c([2H])c(-c4c5ccccc5c(-c5c([2H])c([2H])c6c(oc7c([2H])c8c([2H])c([2H])c([2H])c([2H])c8c([2H])c76)c5[2H])c5ccccc45)c([2H])c3[2H])cc2)c([2H])c1[2H] ; [2H]c1c([2H])c([2H])c(-c2ccc(-c3c([2H])c([2H])c(-c4c5ccccc5c(-c5ccc6oc7cc8ccccc8cc7c6c5)c5ccccc45)c([2H])c3[2H])cc2)c([2H])c1[2H] ; [2H]c1c([2H])c([2H])c(-c2ccc(-c3c([2H])c([2H])c(-c4c5ccccc5c(-c5ccc6oc7c8ccccc8ccc7c6c5)c5ccccc45)c([2H])c3[2H])cc2)c([2H])c1[2H]]

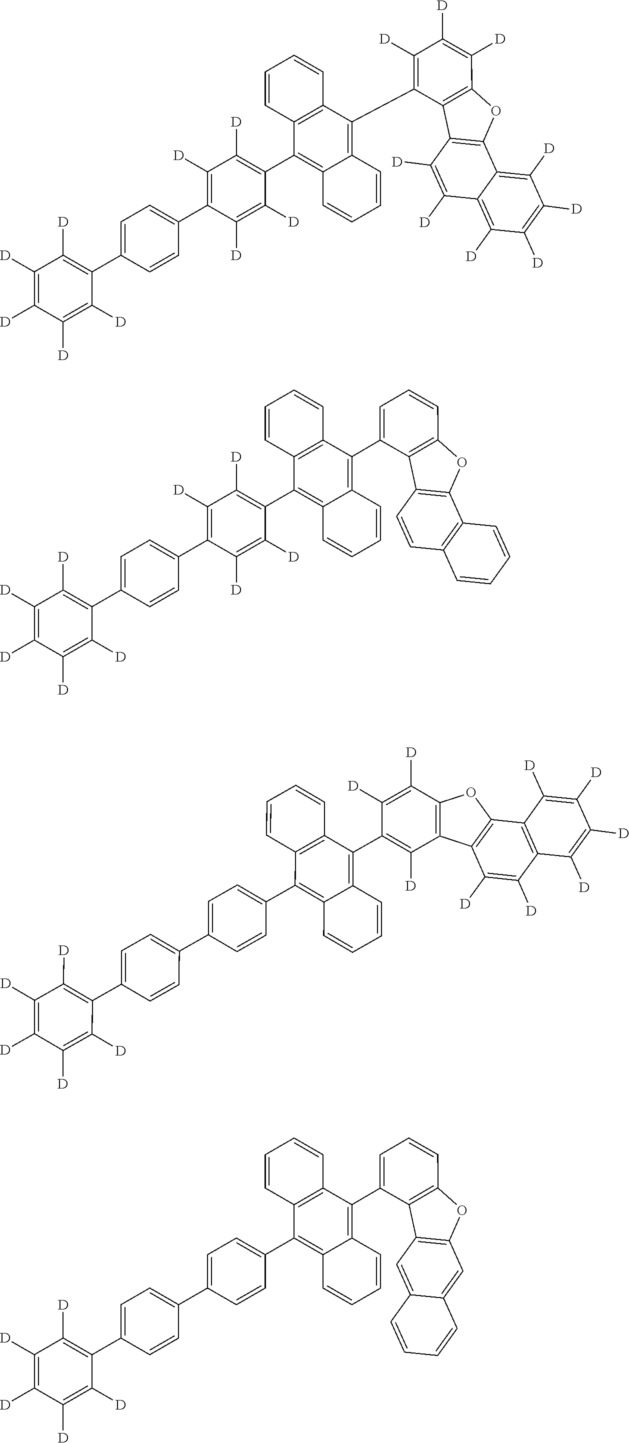

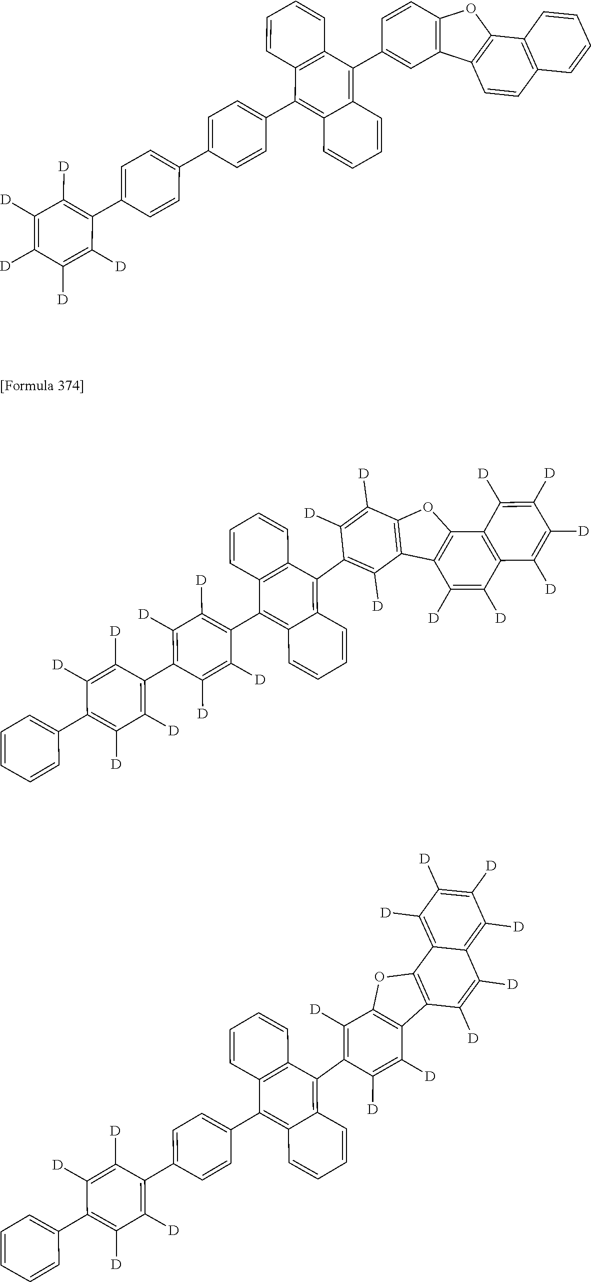 [SMILES: [2H]c1c([2H])c([2H])c(-c2ccc(-c3ccc(-c4c5ccccc5c(-c5ccc6oc7c8ccccc8ccc7c6c5)c5ccccc45)cc3)cc2)c([2H])c1[2H] ; [2H]c1c([2H])c(-c2c([2H])c([2H])c(-c3c4ccccc4c(-c4c([2H])c([2H])c5oc6c7c([2H])c([2H])c([2H])c([2H])c7c([2H])c([2H])c6c5c4[2H])c4ccccc34)c([2H])c2[2H])c([2H])c([2H])c1-c1ccccc1 ; [2H]c1c([2H])c(-c2ccc(-c3c4ccccc4c(-c4c([2H])c([2H])c5c(oc6c7c([2H])c([2H])c([2H])c([2H])c7c([2H])c([2H])c65)c4[2H])c4ccccc34)cc2)c([2H])c([2H])c1-c1ccccc1]

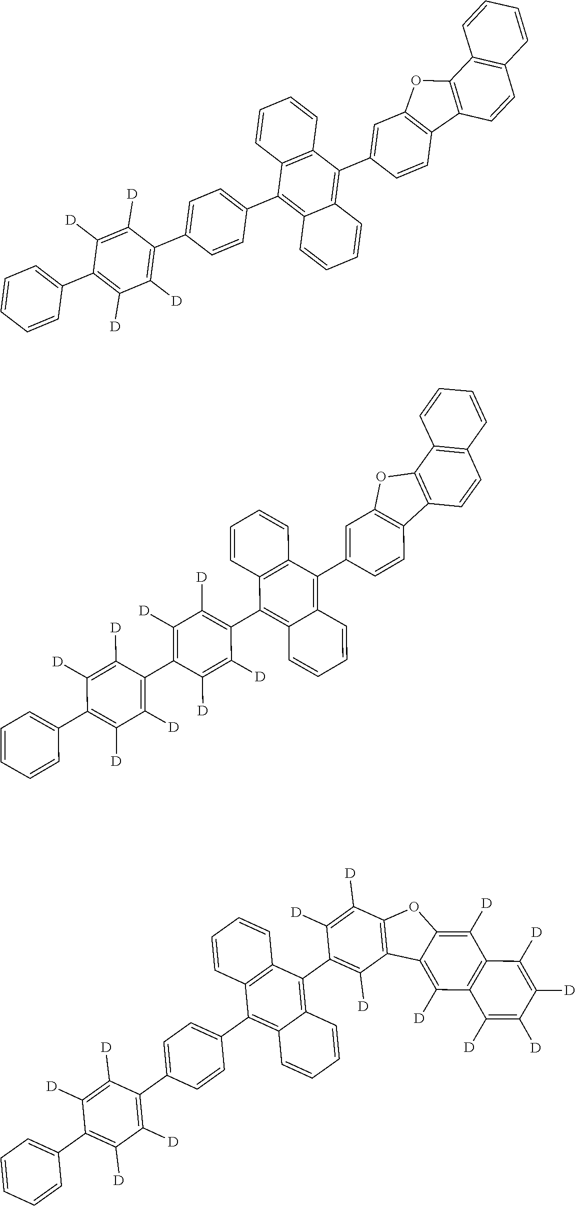 [SMILES: [2H]c1c([2H])c(-c2ccc(-c3c4ccccc4c(-c4ccc5c(c4)oc4c6ccccc6ccc54)c4ccccc34)cc2)c([2H])c([2H])c1-c1ccccc1 ; [2H]c1c([2H])c(-c2c([2H])c([2H])c(-c3c4ccccc4c(-c4ccc5c(c4)oc4c6ccccc6ccc54)c4ccccc34)c([2H])c2[2H])c([2H])c([2H])c1-c1ccccc1 ; [2H]c1c([2H])c(-c2ccc(-c3c4ccccc4c(-c4c([2H])c([2H])c5oc6c([2H])c7c([2H])c([2H])c([2H])c([2H])c7c([2H])c6c5c4[2H])c4ccccc34)cc2)c([2H])c([2H])c1-c1ccccc1]

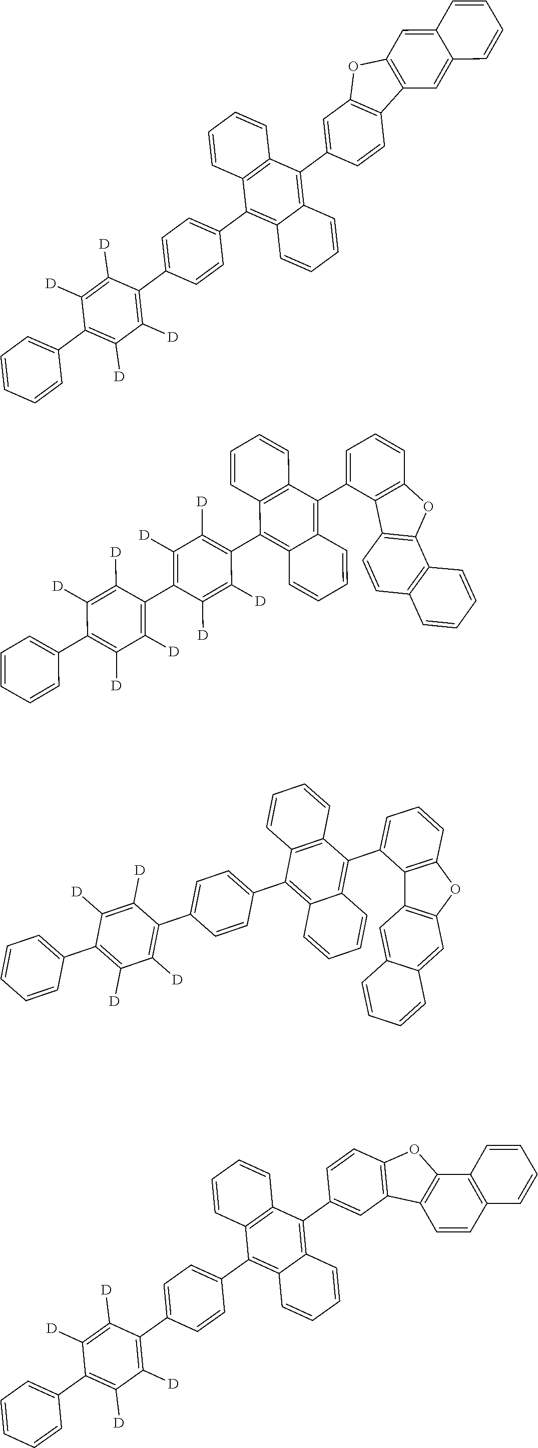 [SMILES: [2H]c1c([2H])c(-c2ccc(-c3c4ccccc4c(-c4ccc5c(c4)oc4cc6ccccc6cc45)c4ccccc34)cc2)c([2H])c([2H])c1-c1ccccc1 ; [2H]c1c([2H])c(-c2c([2H])c([2H])c(-c3c4ccccc4c(-c4cccc5oc6c7ccccc7ccc6c45)c4ccccc34)c([2H])c2[2H])c([2H])c([2H])c1-c1ccccc1 ; [2H]c1c([2H])c(-c2ccc(-c3c4ccccc4c(-c4cccc5oc6cc7ccccc7cc6c45)c4ccccc34)cc2)c([2H])c([2H])c1-c1ccccc1 ; [2H]c1c([2H])c(-c2ccc(-c3c4ccccc4c(-c4ccc5oc6c7ccccc7ccc6c5c4)c4ccccc34)cc2)c([2H])c([2H])c1-c1ccccc1]

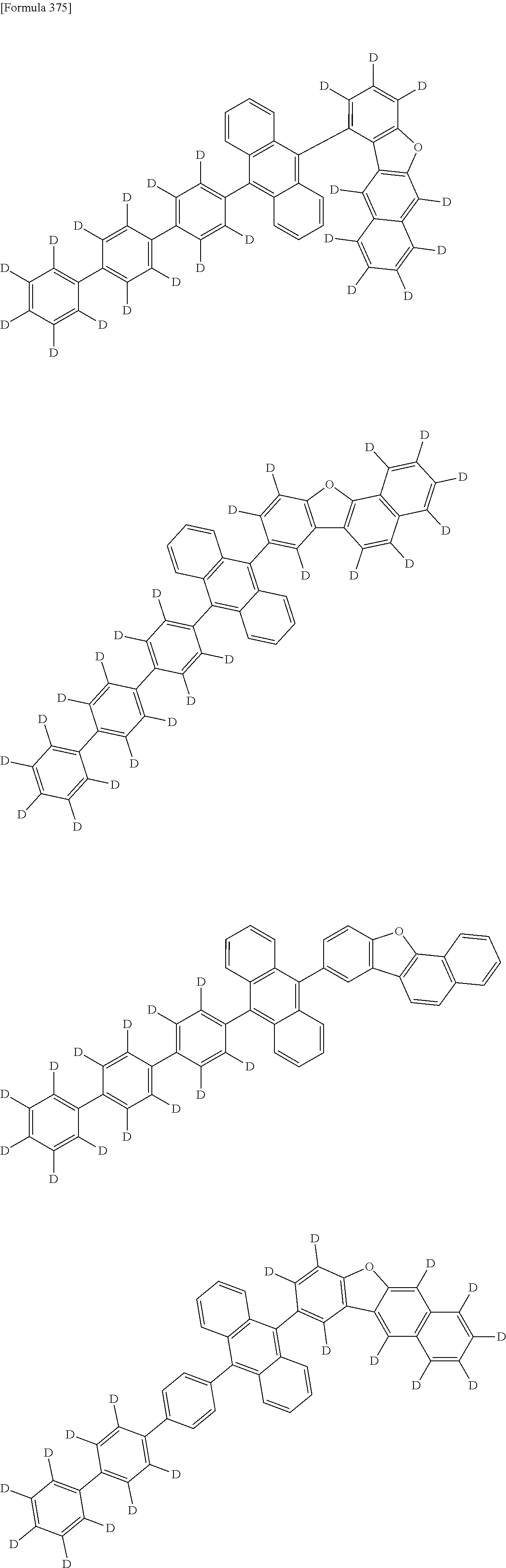

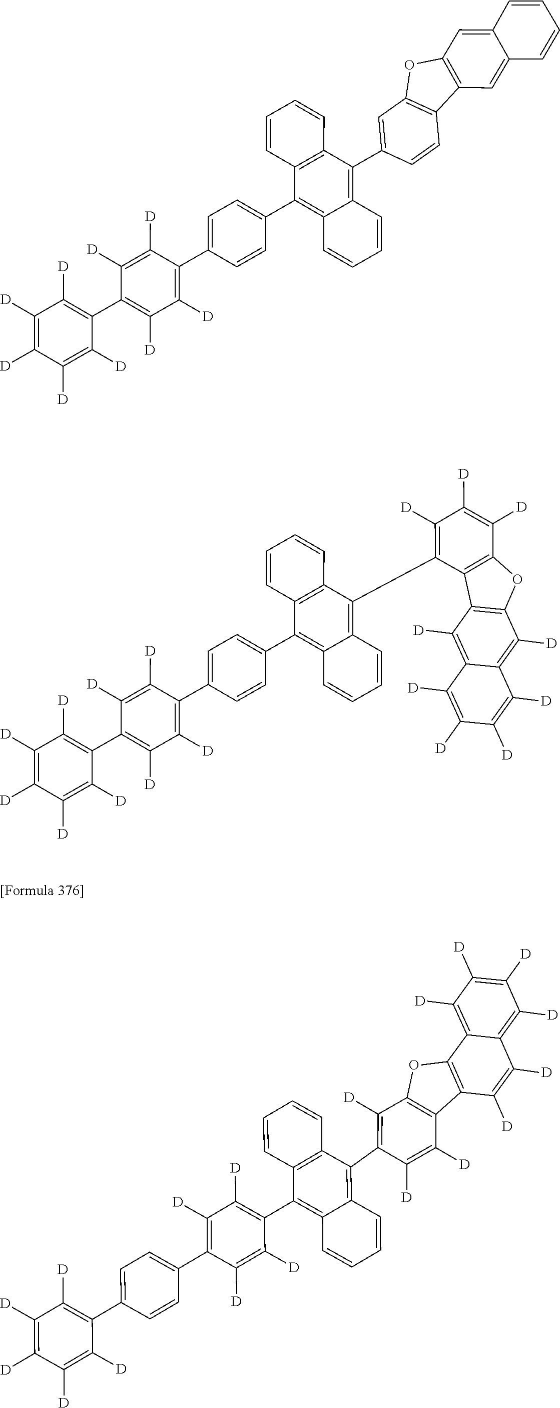

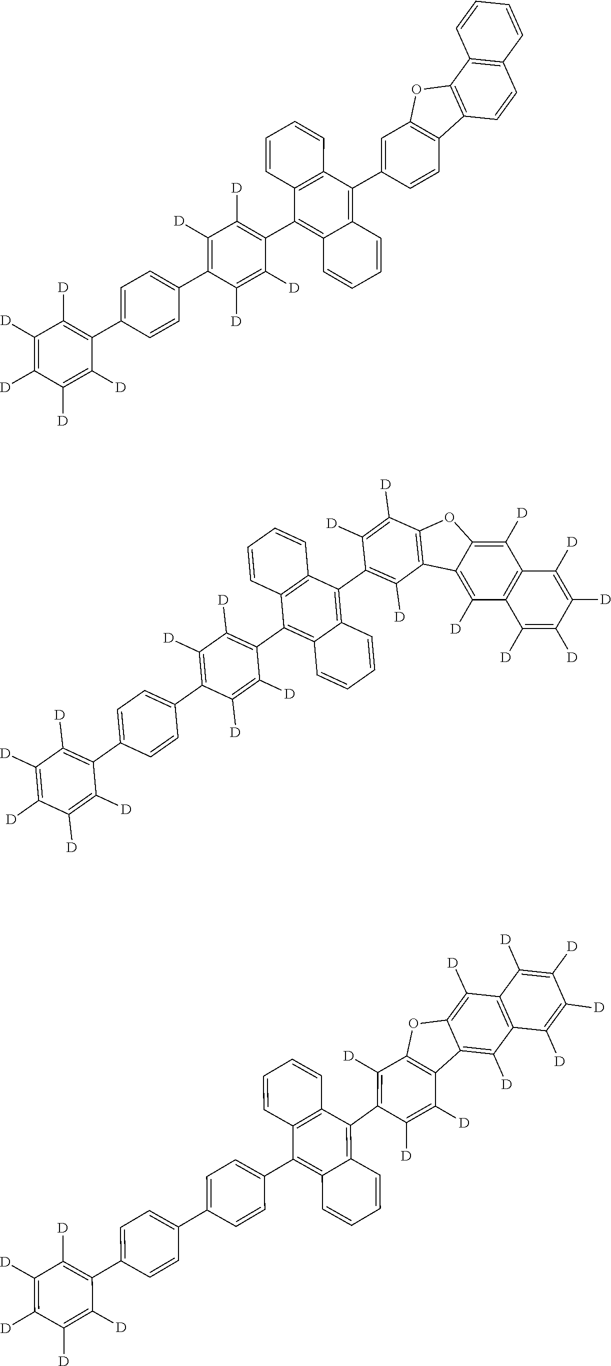 [SMILES: [2H]c1c([2H])c([2H])c(-c2ccc(-c3c([2H])c([2H])c(-c4c5ccccc5c(-c5ccc6c(c5)oc5c7ccccc7ccc65)c5ccccc45)c([2H])c3[2H])cc2)c([2H])c1[2H] ; [2H]c1c([2H])c([2H])c(-c2ccc(-c3c([2H])c([2H])c(-c4c5ccccc5c(-c5c([2H])c([2H])c6oc7c([2H])c8c([2H])c([2H])c([2H])c([2H])c8c([2H])c7c6c5[2H])c5ccccc45)c([2H])c3[2H])cc2)c([2H])c1[2H] ; [2H]c1c([2H])c([2H])c(-c2ccc(-c3ccc(-c4c5ccccc5c(-c5c([2H])c([2H])c6c(oc7c([2H])c8c([2H])c([2H])c([2H])c([2H])c8c([2H])c76)c5[2H])c5ccccc45)cc3)cc2)c([2H])c1[2H]]

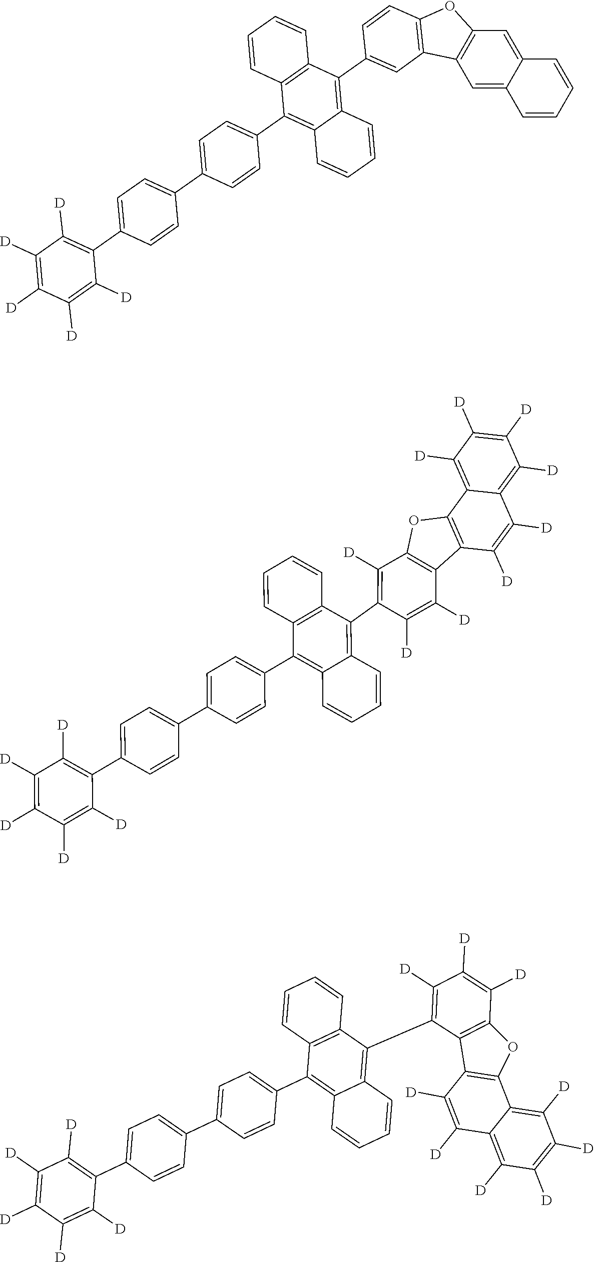 [SMILES: [2H]c1c([2H])c([2H])c(-c2ccc(-c3ccc(-c4c5ccccc5c(-c5ccc6oc7cc8ccccc8cc7c6c5)c5ccccc45)cc3)cc2)c([2H])c1[2H] ; [2H]c1c([2H])c([2H])c(-c2ccc(-c3ccc(-c4c5ccccc5c(-c5c([2H])c([2H])c6c(oc7c8c([2H])c([2H])c([2H])c([2H])c8c([2H])c([2H])c76)c5[2H])c5ccccc45)cc3)cc2)c([2H])c1[2H] ; [2H]c1c([2H])c([2H])c(-c2ccc(-c3ccc(-c4c5ccccc5c(-c5c([2H])c([2H])c([2H])c6oc7c8c([2H])c([2H])c([2H])c([2H])c8c([2H])c([2H])c7c56)c5ccccc45)cc3)cc2)c([2H])c1[2H]]

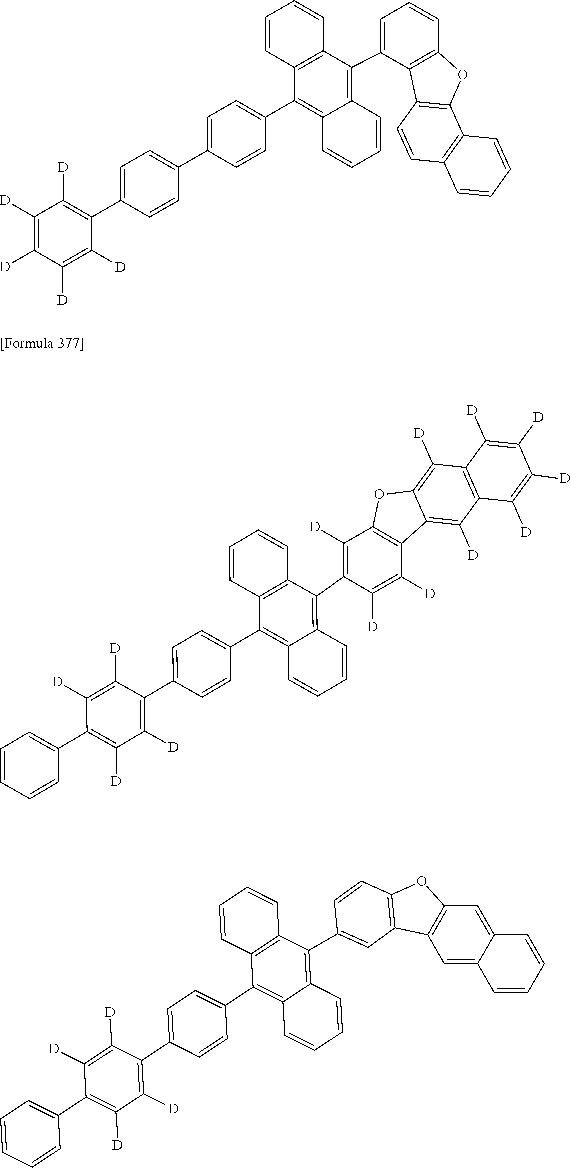 [SMILES: [2H]c1c([2H])c([2H])c(-c2ccc(-c3ccc(-c4c5ccccc5c(-c5cccc6oc7c8ccccc8ccc7c56)c5ccccc45)cc3)cc2)c([2H])c1[2H] ; [2H]c1c([2H])c(-c2ccc(-c3c4ccccc4c(-c4c([2H])c([2H])c5c(oc6c([2H])c7c([2H])c([2H])c([2H])c([2H])c7c([2H])c65)c4[2H])c4ccccc34)cc2)c([2H])c([2H])c1-c1ccccc1 ; [2H]c1c([2H])c(-c2ccc(-c3c4ccccc4c(-c4ccc5oc6cc7ccccc7cc6c5c4)c4ccccc34)cc2)c([2H])c([2H])c1-c1ccccc1]

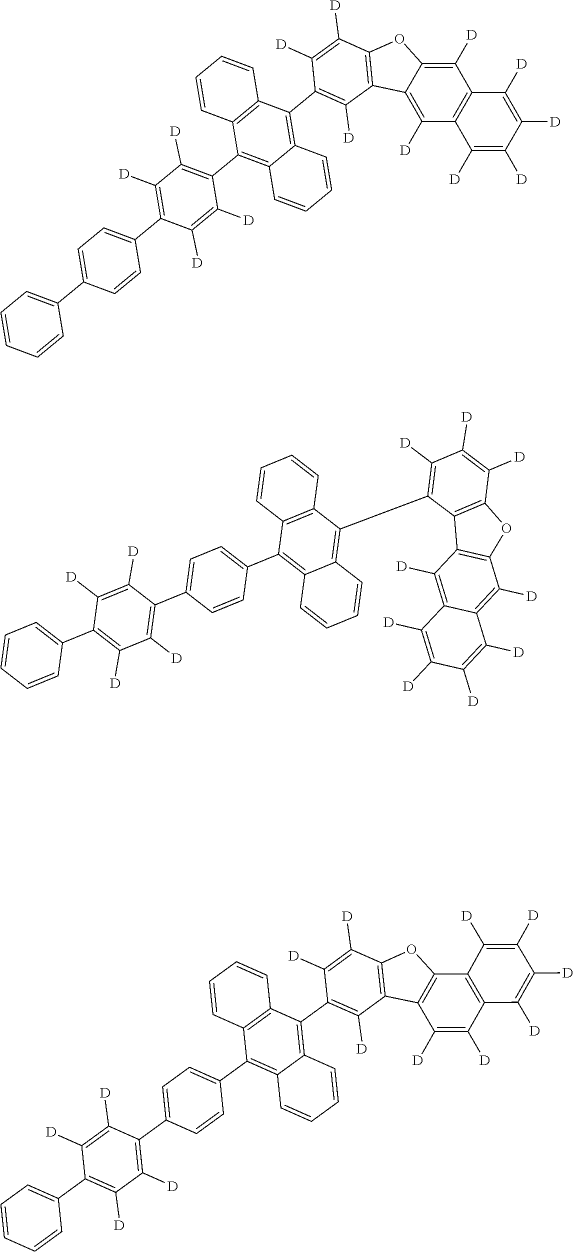 [SMILES: [2H]c1c([2H])c(-c2c3ccccc3c(-c3c([2H])c([2H])c4oc5c([2H])c6c([2H])c([2H])c([2H])c([2H])c6c([2H])c5c4c3[2H])c3ccccc23)c([2H])c([2H])c1-c1ccc(-c2ccccc2)cc1 ; [2H]c1c([2H])c(-c2ccc(-c3c4ccccc4c(-c4c([2H])c([2H])c([2H])c5oc6c([2H])c7c([2H])c([2H])c([2H])c([2H])c7c([2H])c6c45)c4ccccc34)cc2)c([2H])c([2H])c1-c1ccccc1 ; [2H]c1c([2H])c(-c2ccc(-c3c4ccccc4c(-c4c([2H])c([2H])c5oc6c7c([2H])c([2H])c([2H])c([2H])c7c([2H])c([2H])c6c5c4[2H])c4ccccc34)cc2)c([2H])c([2H])c1-c1ccccc1]

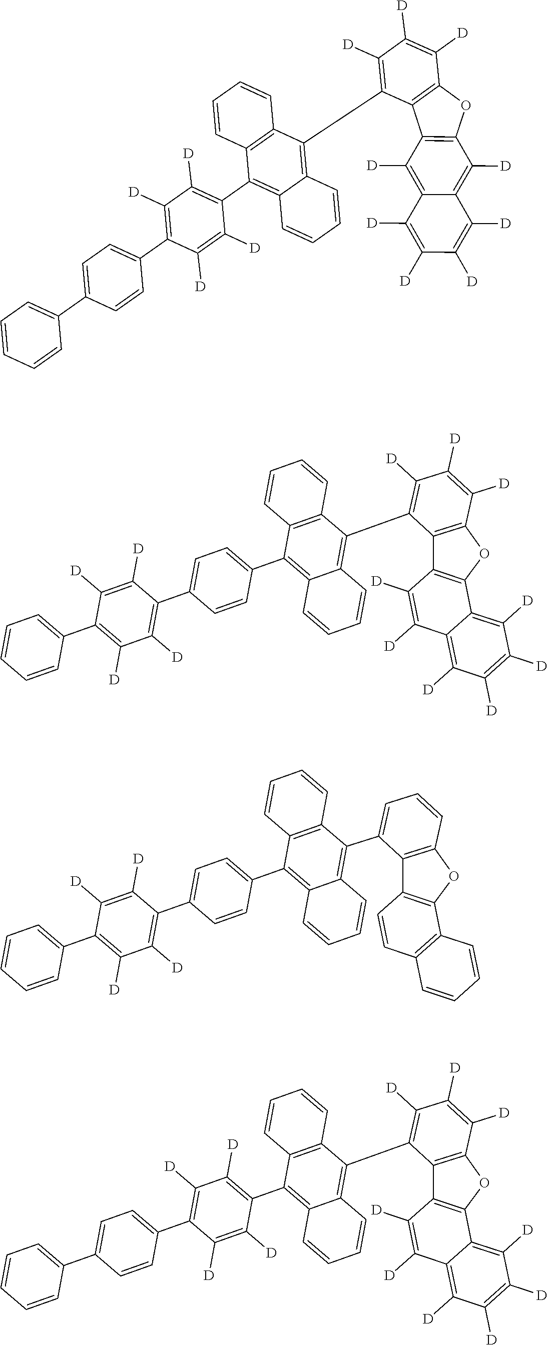

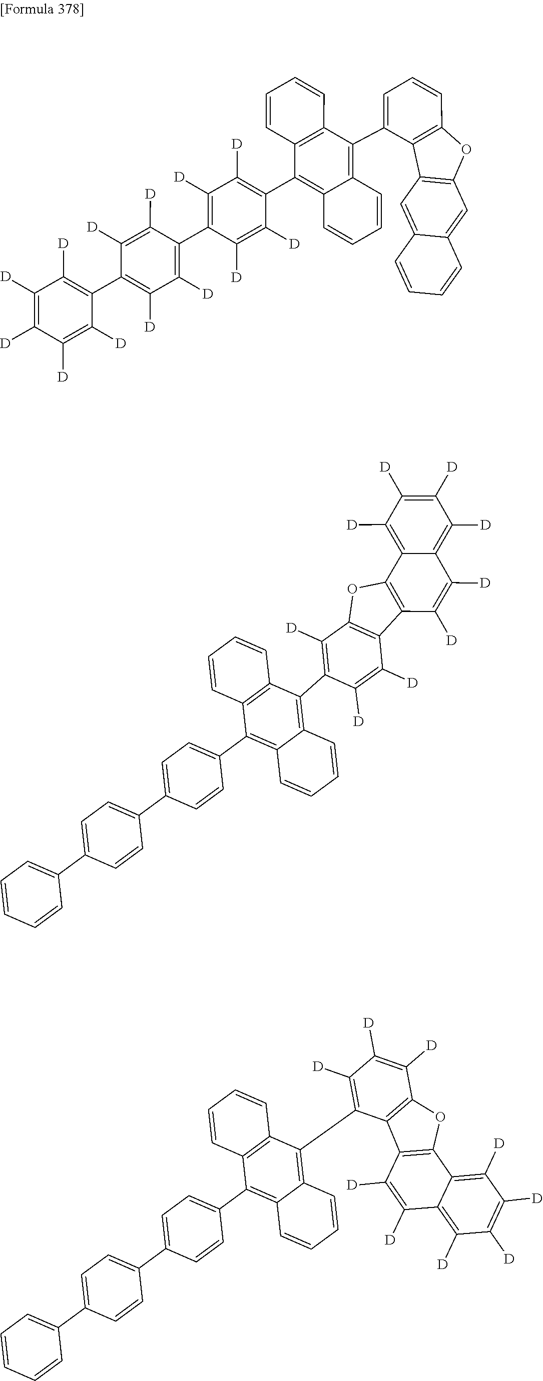 [SMILES: [2H]c1c([2H])c([2H])c(-c2c([2H])c([2H])c(-c3c([2H])c([2H])c(-c4c5ccccc5c(-c5cccc6oc7cc8ccccc8cc7c56)c5ccccc45)c([2H])c3[2H])c([2H])c2[2H])c([2H])c1[2H] ; [2H]c1c([2H])c([2H])c2c(c1[2H])c([2H])c([2H])c1c2oc2c([2H])c(-c3c4ccccc4c(-c4ccc(-c5ccc(-c6ccccc6)cc5)cc4)c4ccccc34)c([2H])c([2H])c21 ; [2H]c1c([2H])c(-c2c3ccccc3c(-c3ccc(-c4ccc(-c5ccccc5)cc4)cc3)c3ccccc23)c2c(oc3c4c([2H])c([2H])c([2H])c([2H])c4c([2H])c([2H])c32)c1[2H]]

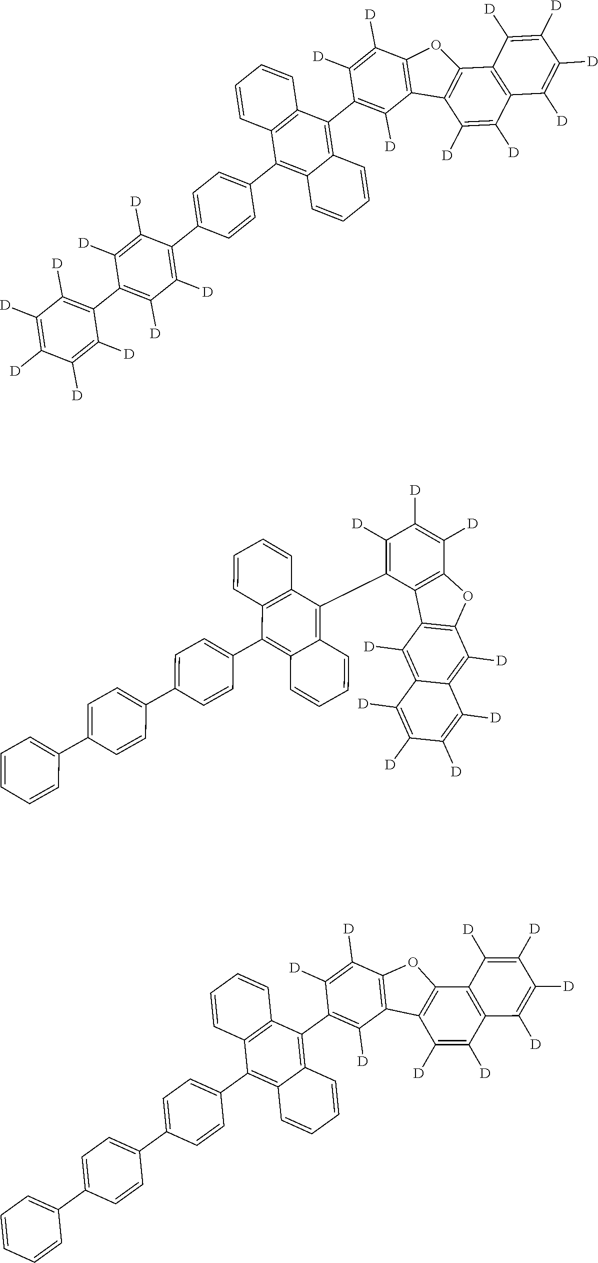 [SMILES: [2H]c1c([2H])c([2H])c(-c2c([2H])c([2H])c(-c3ccc(-c4c5ccccc5c(-c5c([2H])c([2H])c6oc7c8c([2H])c([2H])c([2H])c([2H])c8c([2H])c([2H])c7c6c5[2H])c5ccccc45)cc3)c([2H])c2[2H])c([2H])c1[2H] ; [2H]c1c([2H])c(-c2c3ccccc3c(-c3ccc(-c4ccc(-c5ccccc5)cc4)cc3)c3ccccc23)c2c(oc3c([2H])c4c([2H])c([2H])c([2H])c([2H])c4c([2H])c32)c1[2H] ; [2H]c1c(-c2c3ccccc3c(-c3ccc(-c4ccc(-c5ccccc5)cc4)cc3)c3ccccc23)c([2H])c2c(oc3c4c([2H])c([2H])c([2H])c([2H])c4c([2H])c([2H])c32)c1[2H]]

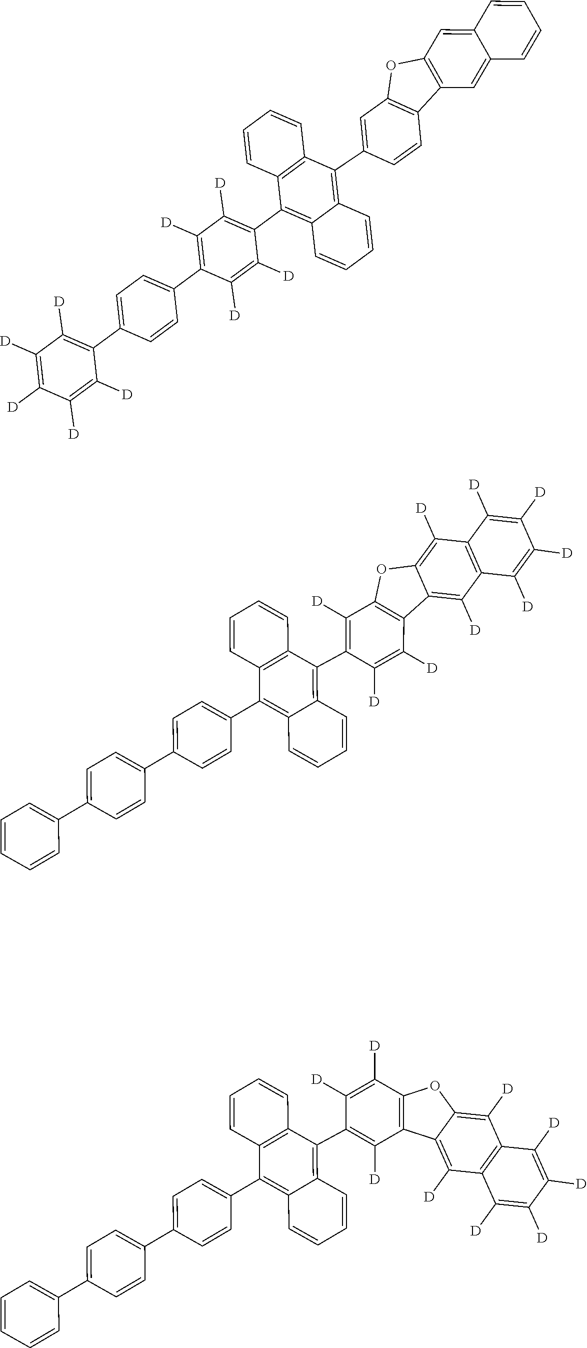 [SMILES: [2H]c1c([2H])c([2H])c(-c2ccc(-c3c([2H])c([2H])c(-c4c5ccccc5c(-c5ccc6c(c5)oc5cc7ccccc7cc56)c5ccccc45)c([2H])c3[2H])cc2)c([2H])c1[2H] ; [2H]c1c([2H])c([2H])c2c([2H])c3c(oc4c([2H])c(-c5c6ccccc6c(-c6ccc(-c7ccc(-c8ccccc8)cc7)cc6)c6ccccc56)c([2H])c([2H])c43)c([2H])c2c1[2H] ; [2H]c1c(-c2c3ccccc3c(-c3ccc(-c4ccc(-c5ccccc5)cc4)cc3)c3ccccc23)c([2H])c2c(oc3c([2H])c4c([2H])c([2H])c([2H])c([2H])c4c([2H])c32)c1[2H]]

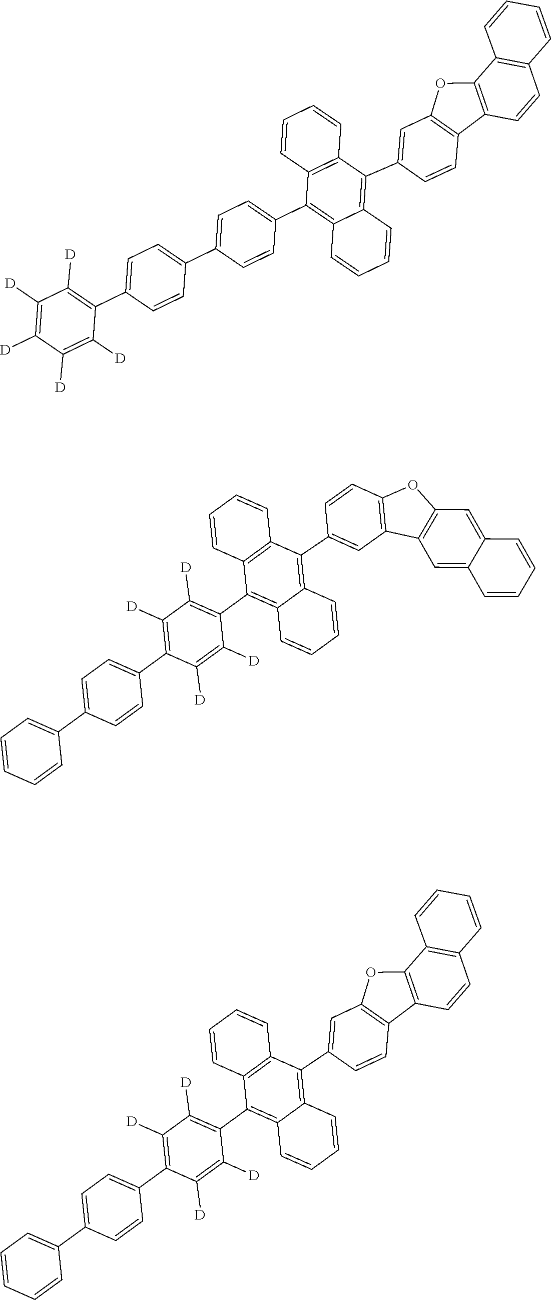 [SMILES: [2H]c1c([2H])c([2H])c(-c2ccc(-c3ccc(-c4c5ccccc5c(-c5ccc6c(c5)oc5c7ccccc7ccc65)c5ccccc45)cc3)cc2)c([2H])c1[2H] ; [2H]c1c([2H])c(-c2c3ccccc3c(-c3ccc4oc5cc6ccccc6cc5c4c3)c3ccccc23)c([2H])c([2H])c1-c1ccc(-c2ccccc2)cc1 ; [2H]c1c([2H])c(-c2c3ccccc3c(-c3ccc4c(c3)oc3c5ccccc5ccc43)c3ccccc23)c([2H])c([2H])c1-c1ccc(-c2ccccc2)cc1]

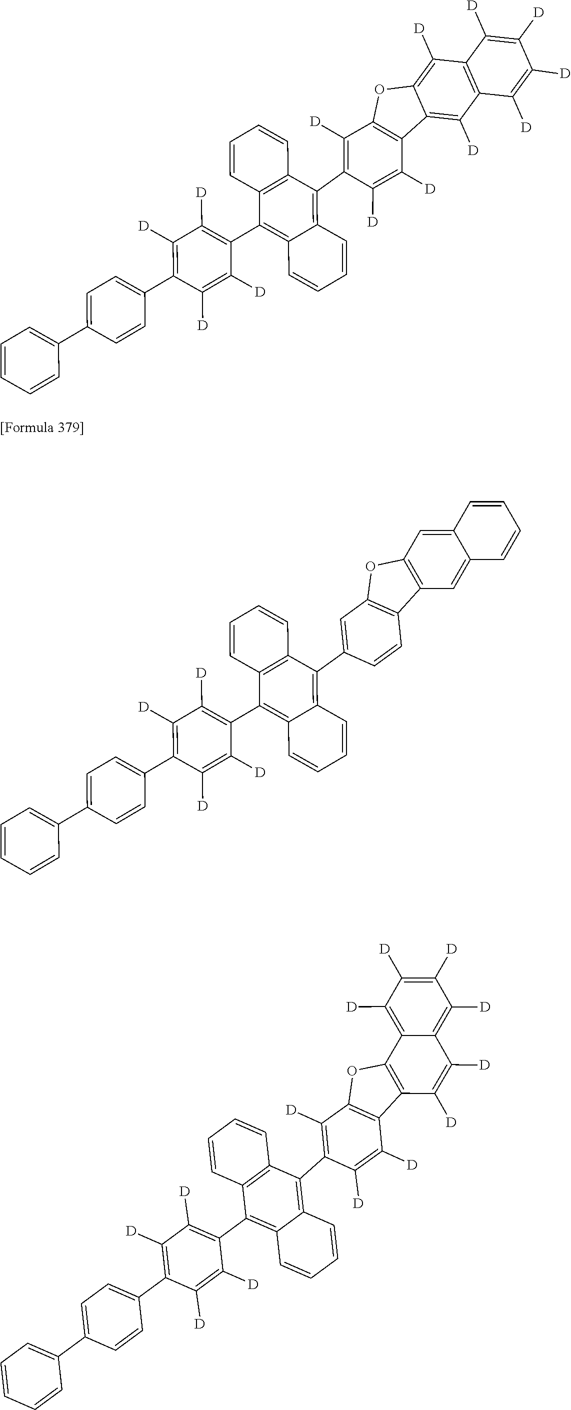 [SMILES: [2H]c1c([2H])c(-c2c3ccccc3c(-c3c([2H])c([2H])c4c(oc5c([2H])c6c([2H])c([2H])c([2H])c([2H])c6c([2H])c54)c3[2H])c3ccccc23)c([2H])c([2H])c1-c1ccc(-c2ccccc2)cc1 ; [2H]c1c([2H])c(-c2c3ccccc3c(-c3ccc4c(c3)oc3cc5ccccc5cc34)c3ccccc23)c([2H])c([2H])c1-c1ccc(-c2ccccc2)cc1 ; [2H]c1c([2H])c(-c2c3ccccc3c(-c3c([2H])c([2H])c4c(oc5c6c([2H])c([2H])c([2H])c([2H])c6c([2H])c([2H])c54)c3[2H])c3ccccc23)c([2H])c([2H])c1-c1ccc(-c2ccccc2)cc1]

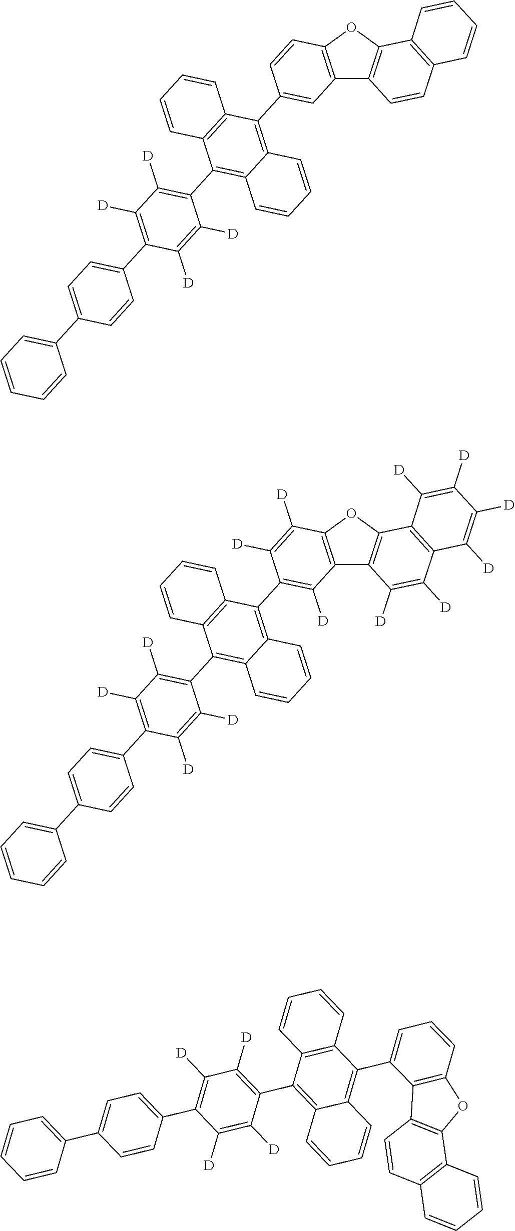 [SMILES: [2H]c1c([2H])c(-c2c3ccccc3c(-c3ccc4oc5c6ccccc6ccc5c4c3)c3ccccc23)c([2H])c([2H])c1-c1ccc(-c2ccccc2)cc1 ; [2H]c1c([2H])c(-c2c3ccccc3c(-c3c([2H])c([2H])c4oc5c6c([2H])c([2H])c([2H])c([2H])c6c([2H])c([2H])c5c4c3[2H])c3ccccc23)c([2H])c([2H])c1-c1ccc(-c2ccccc2)cc1 ; [2H]c1c([2H])c(-c2c3ccccc3c(-c3cccc4oc5c6ccccc6ccc5c34)c3ccccc23)c([2H])c([2H])c1-c1ccc(-c2ccccc2)cc1]

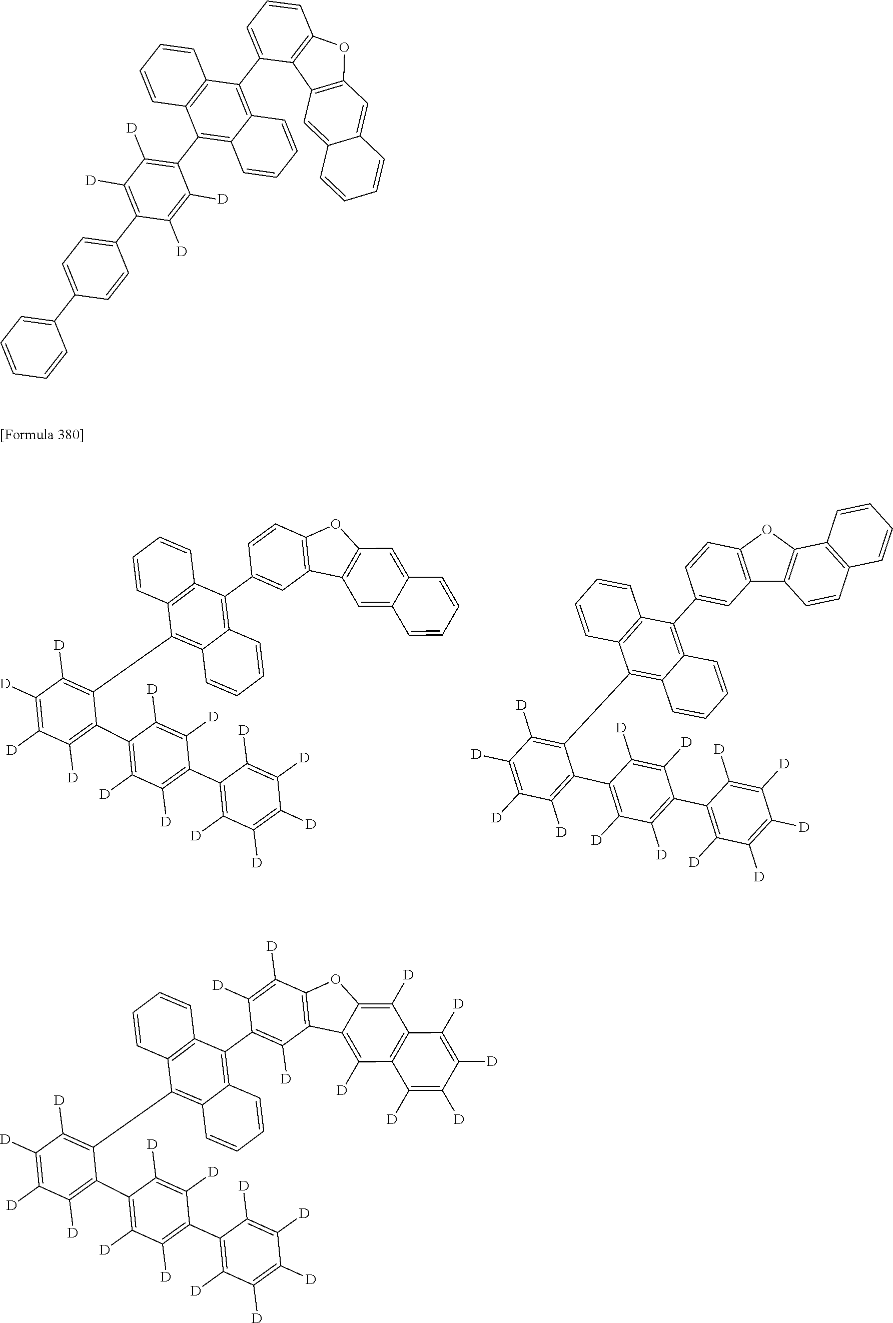

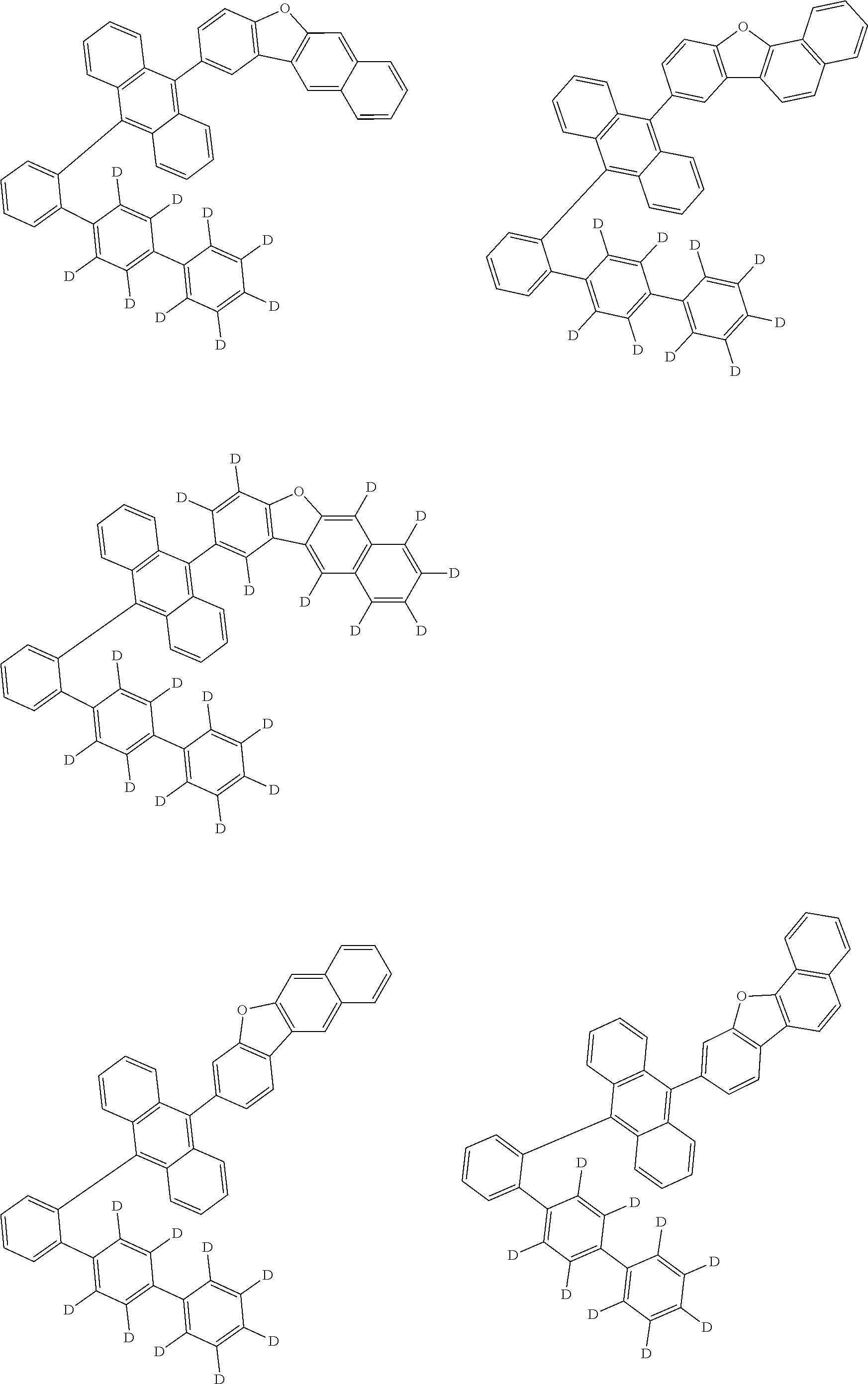

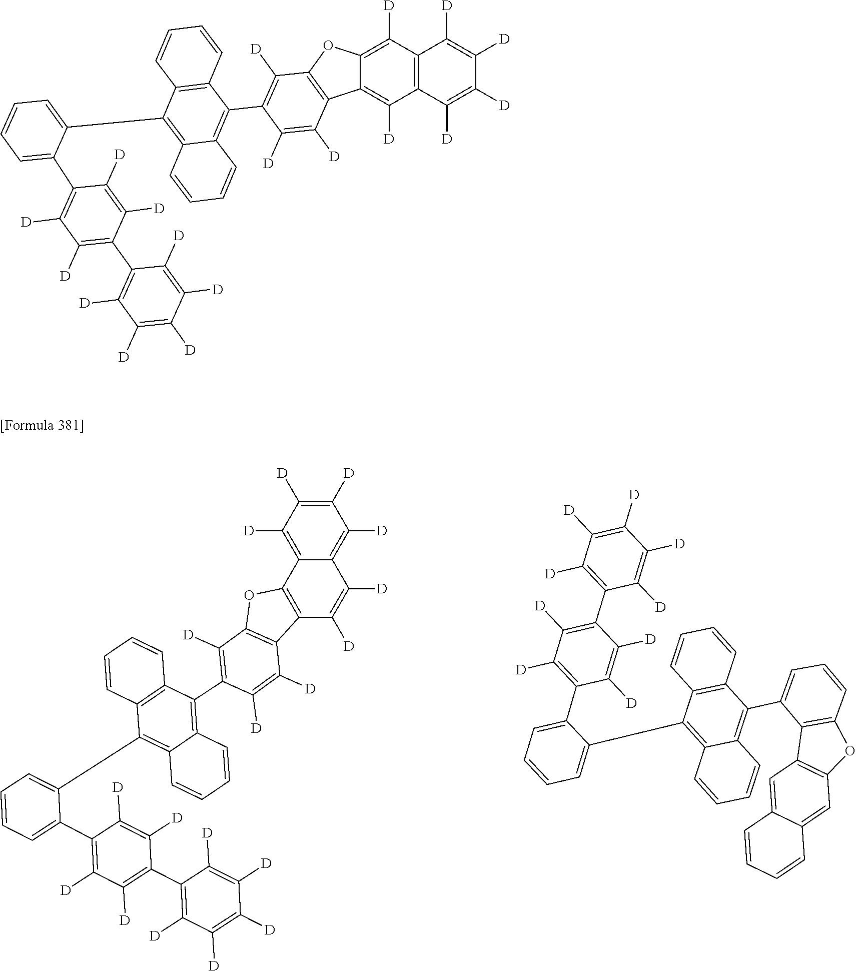 [SMILES: [2H]c1c([2H])c([2H])c(-c2c([2H])c([2H])c(-c3ccccc3-c3c4ccccc4c(-c4c([2H])c([2H])c5c(oc6c([2H])c7c([2H])c([2H])c([2H])c([2H])c7c([2H])c65)c4[2H])c4ccccc34)c([2H])c2[2H])c([2H])c1[2H] ; [2H]c1c([2H])c([2H])c(-c2c([2H])c([2H])c(-c3ccccc3-c3c4ccccc4c(-c4c([2H])c([2H])c5c(oc6c7c([2H])c([2H])c([2H])c([2H])c7c([2H])c([2H])c65)c4[2H])c4ccccc34)c([2H])c2[2H])c([2H])c1[2H] ; [2H]c1c([2H])c([2H])c(-c2c([2H])c([2H])c(-c3ccccc3-c3c4ccccc4c(-c4cccc5oc6cc7ccccc7cc6c45)c4ccccc34)c([2H])c2[2H])c([2H])c1[2H]]

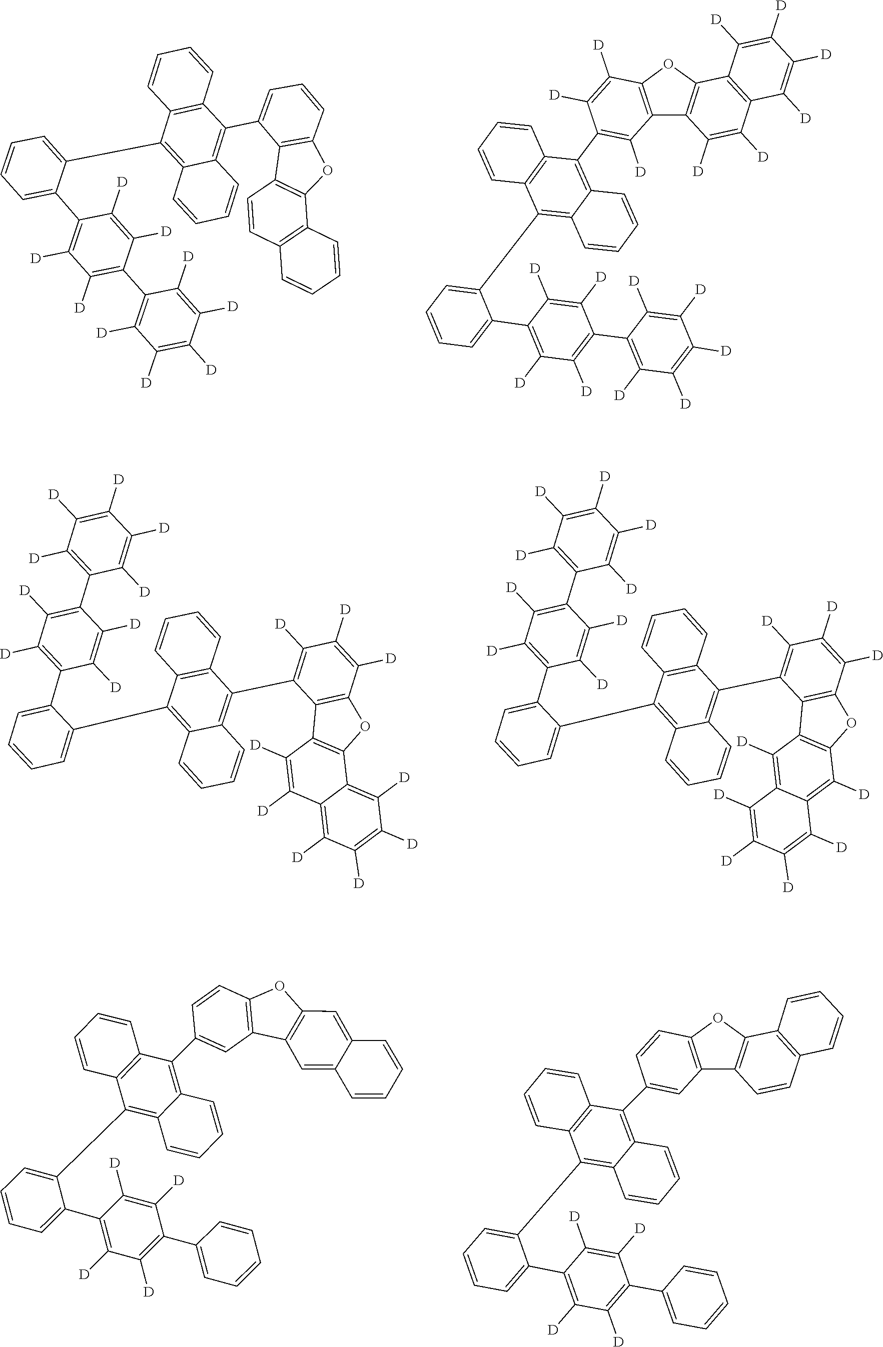

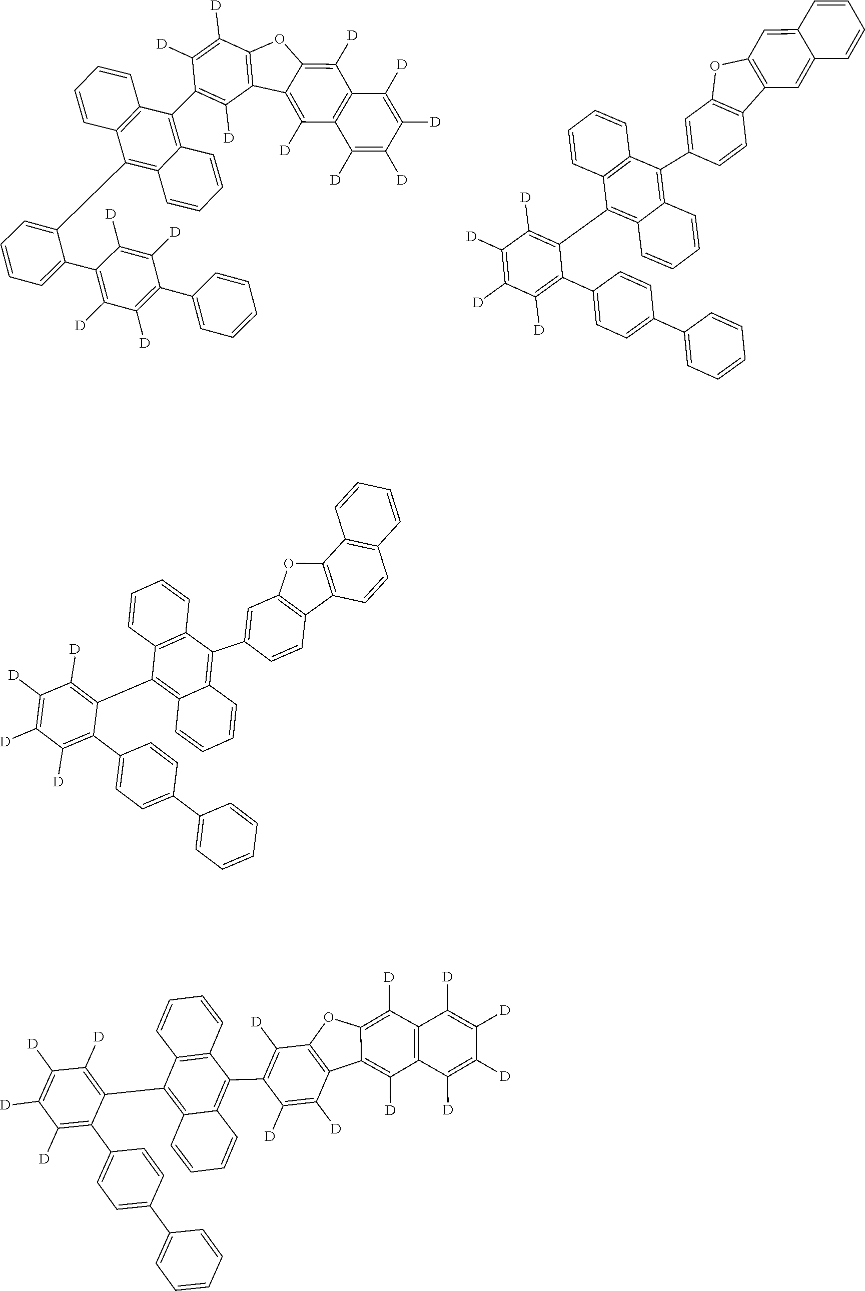

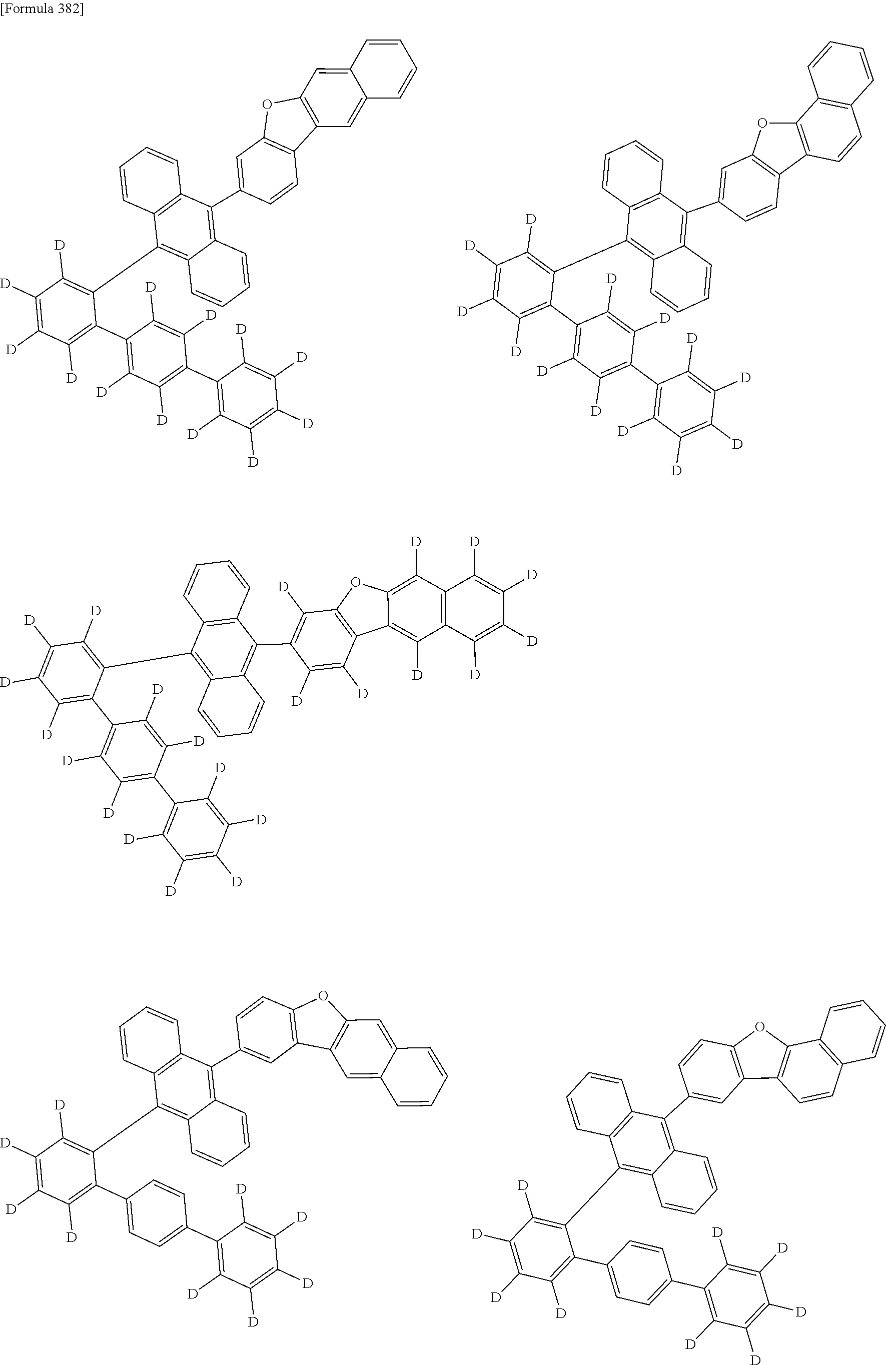

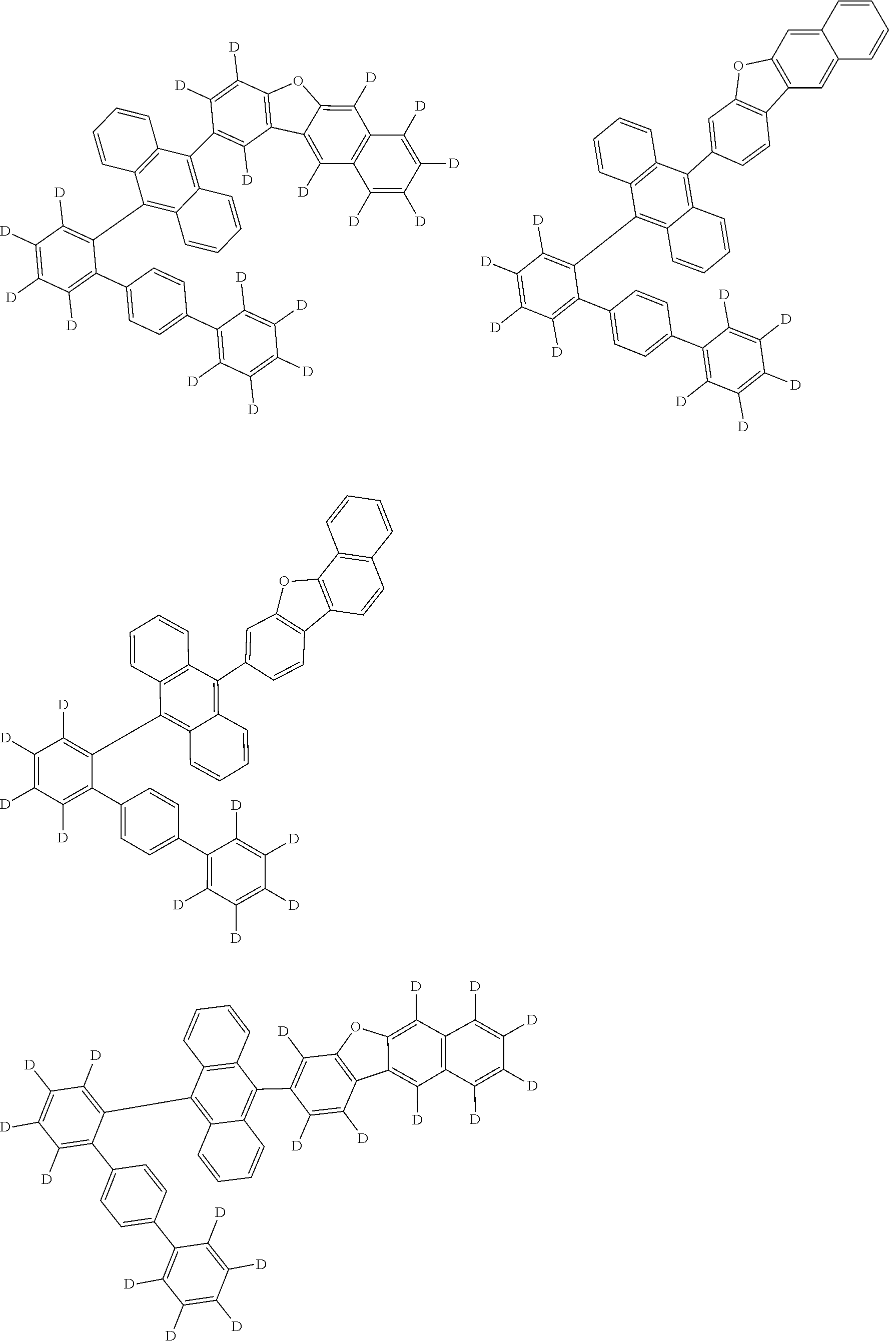

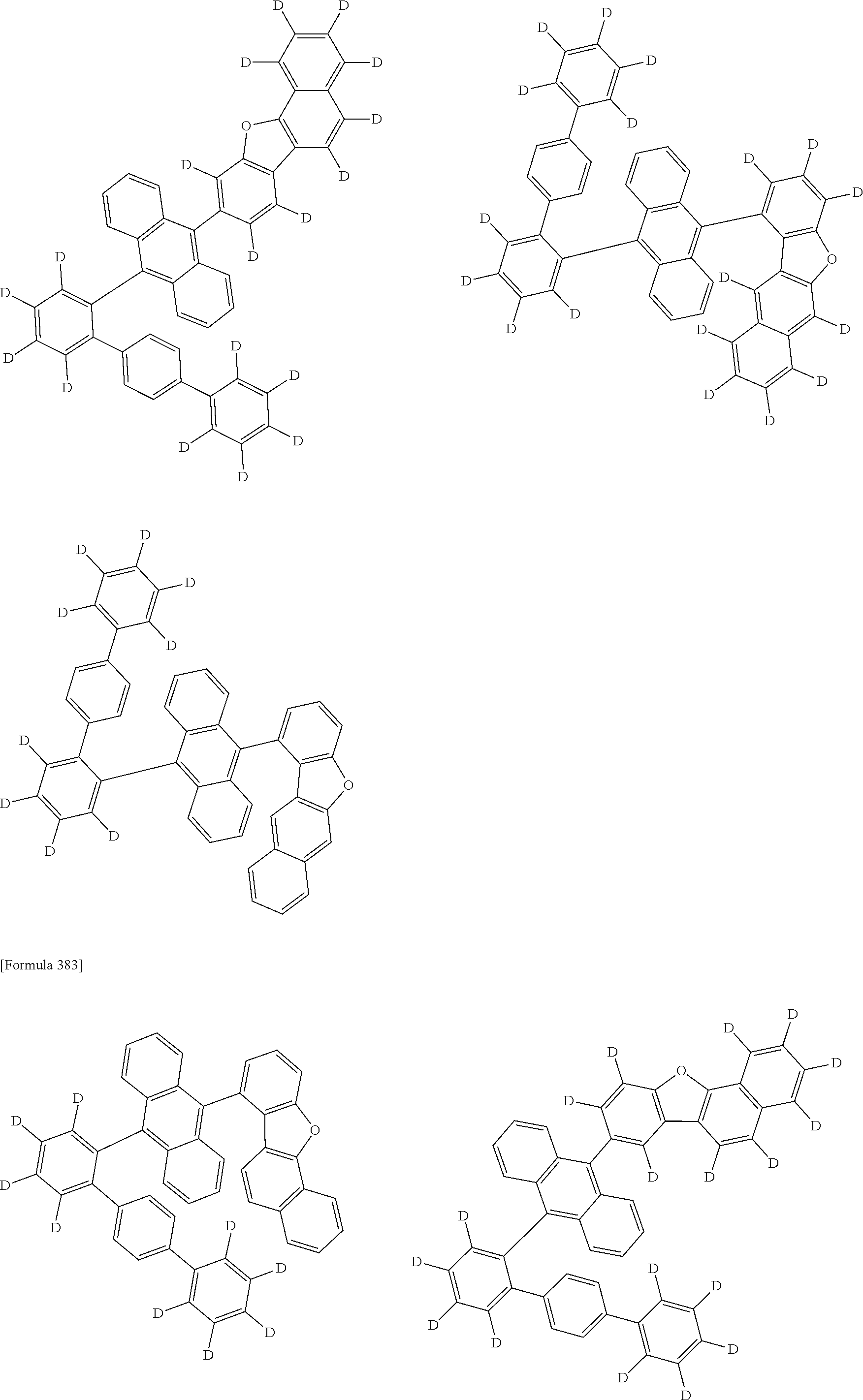

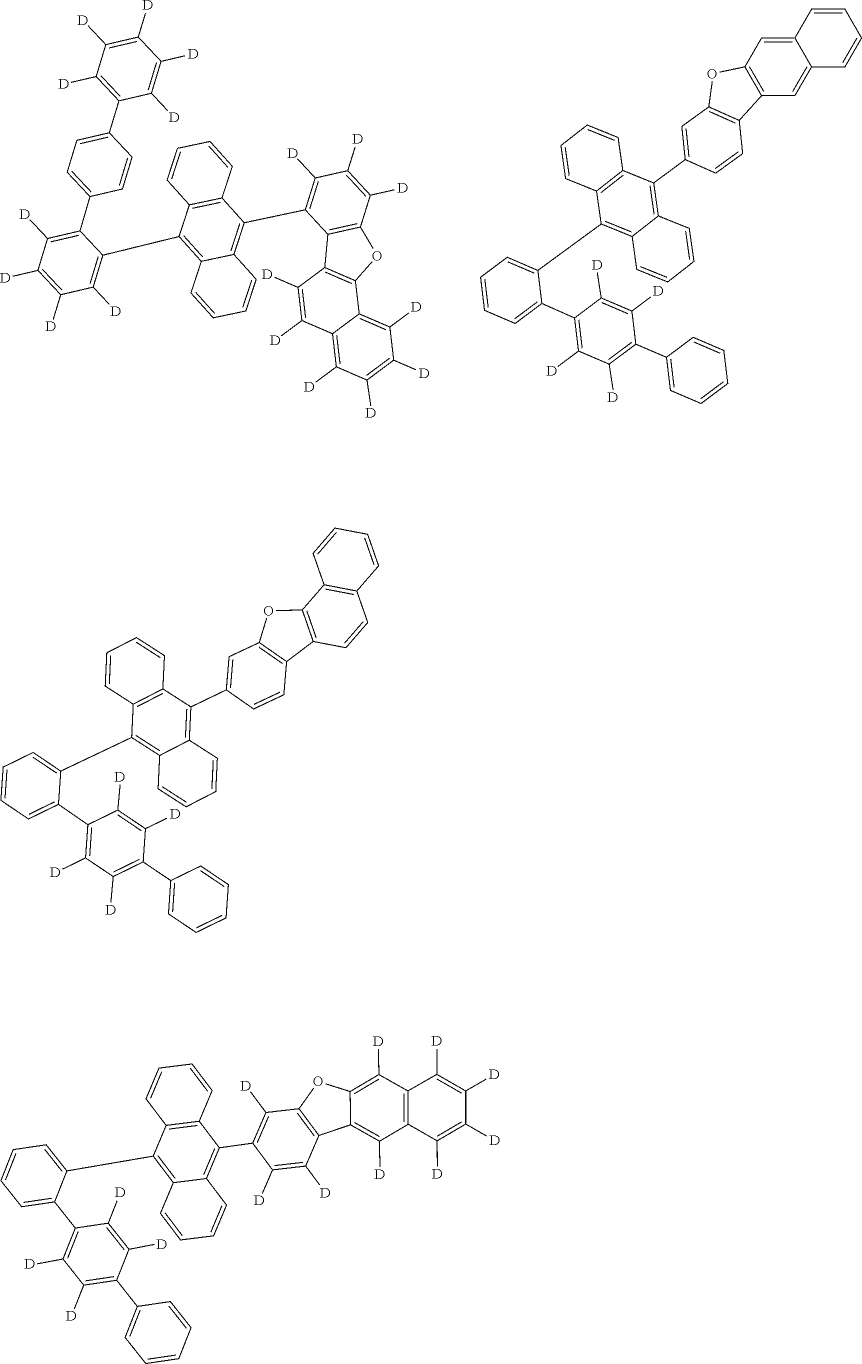

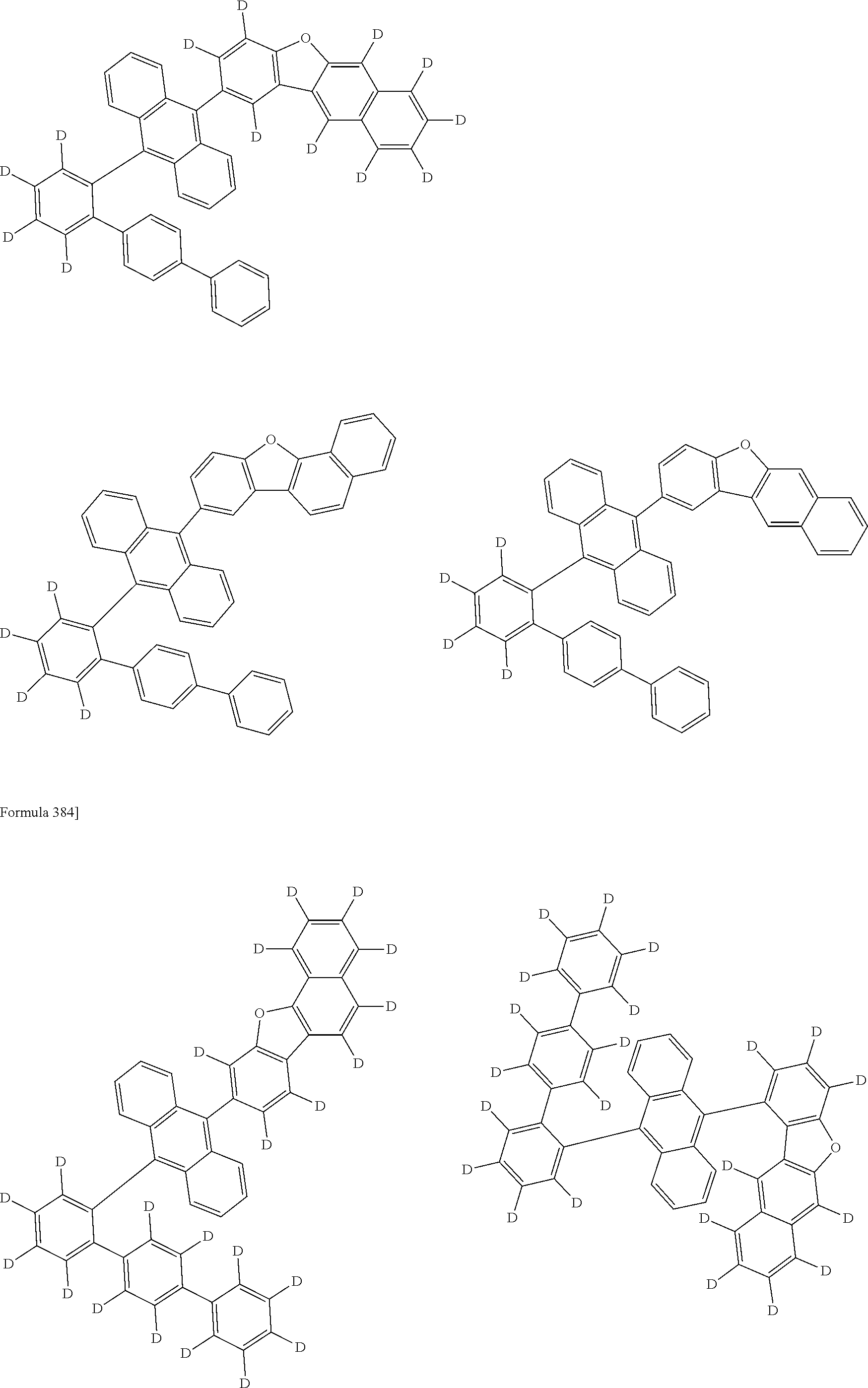

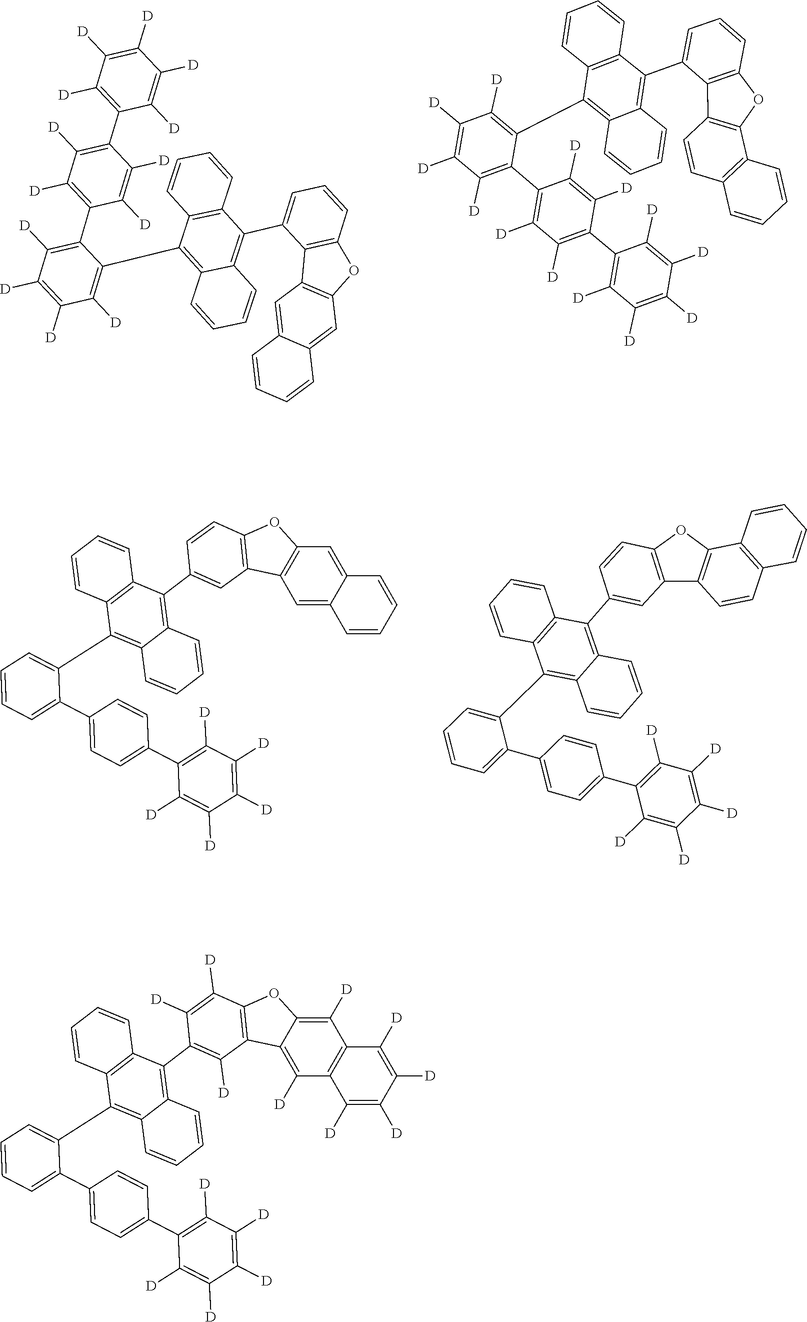

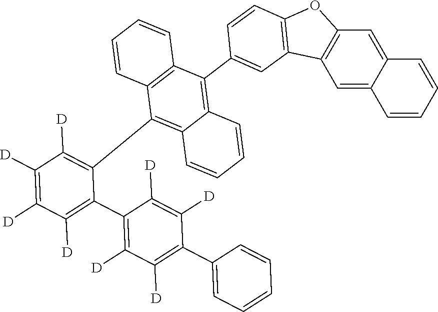 [SMILES: [2H]c1c([2H])c([2H])c(-c2c3ccccc3c(-c3ccc4oc5cc6ccccc6cc5c4c3)c3ccccc23)c(-c2c([2H])c([2H])c(-c3ccccc3)c([2H])c2[2H])c1[2H]]

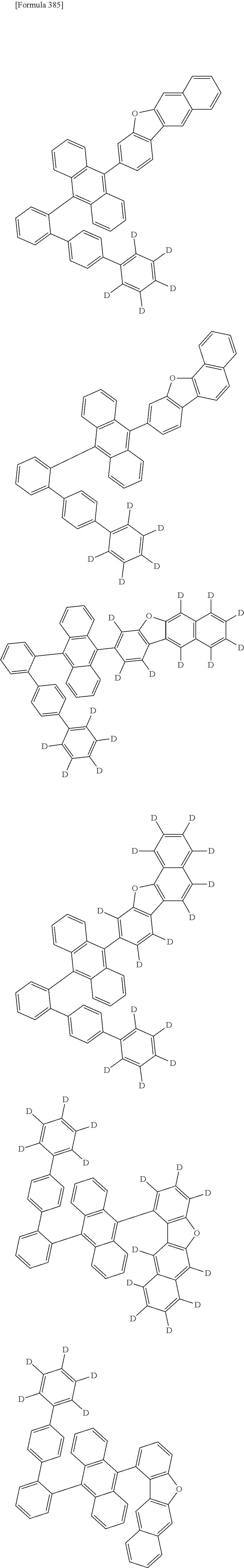

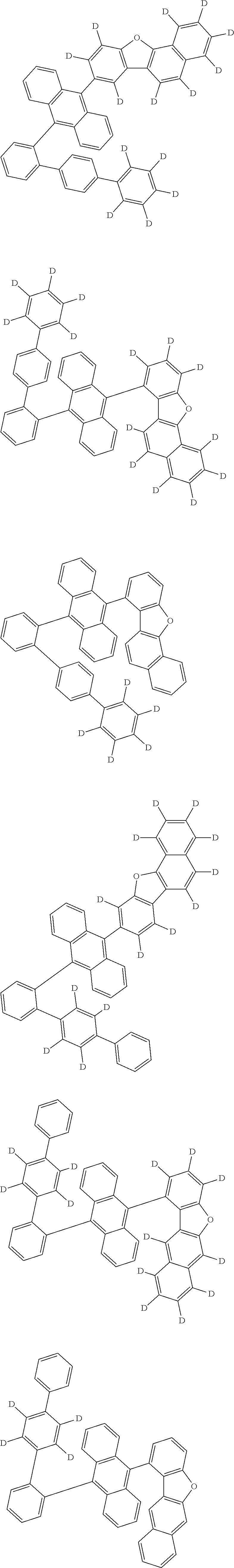

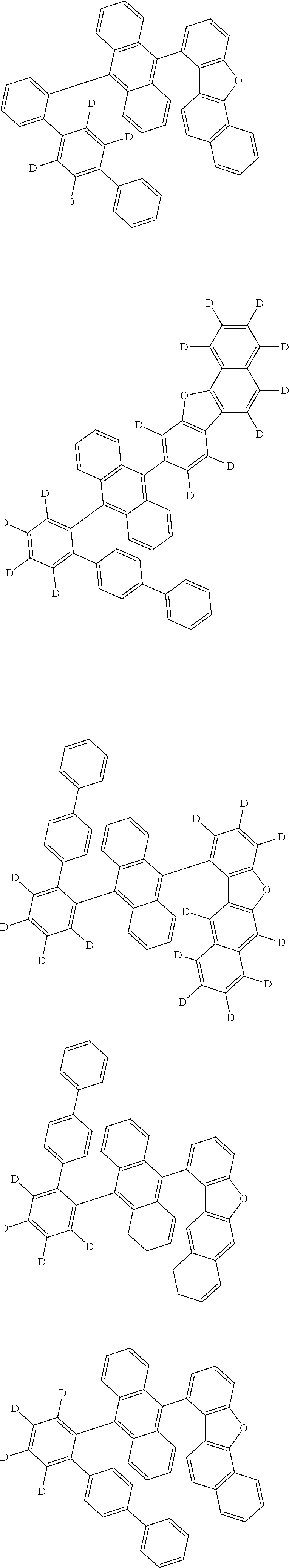

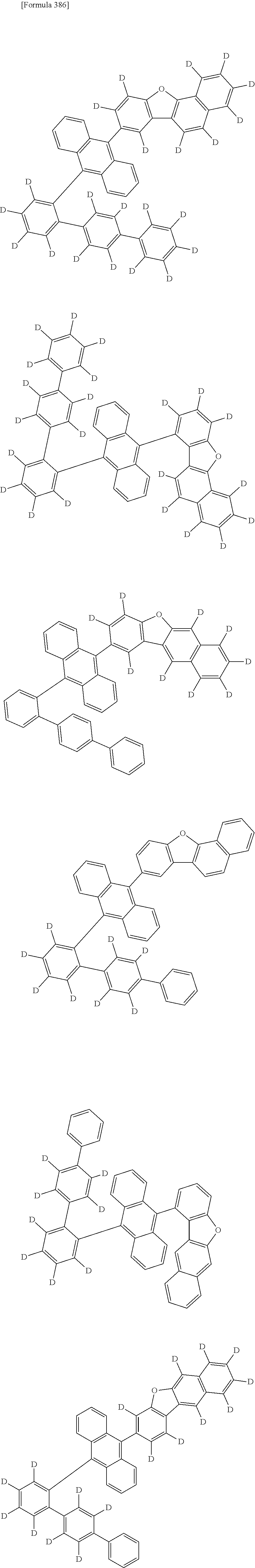

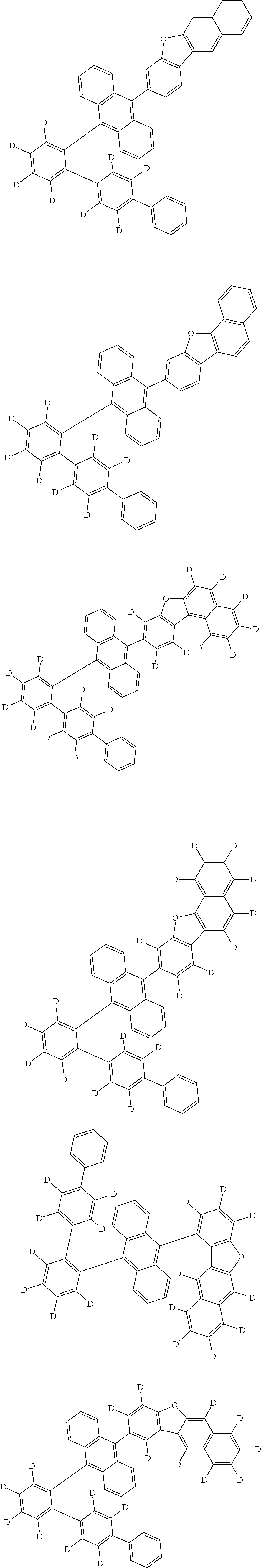

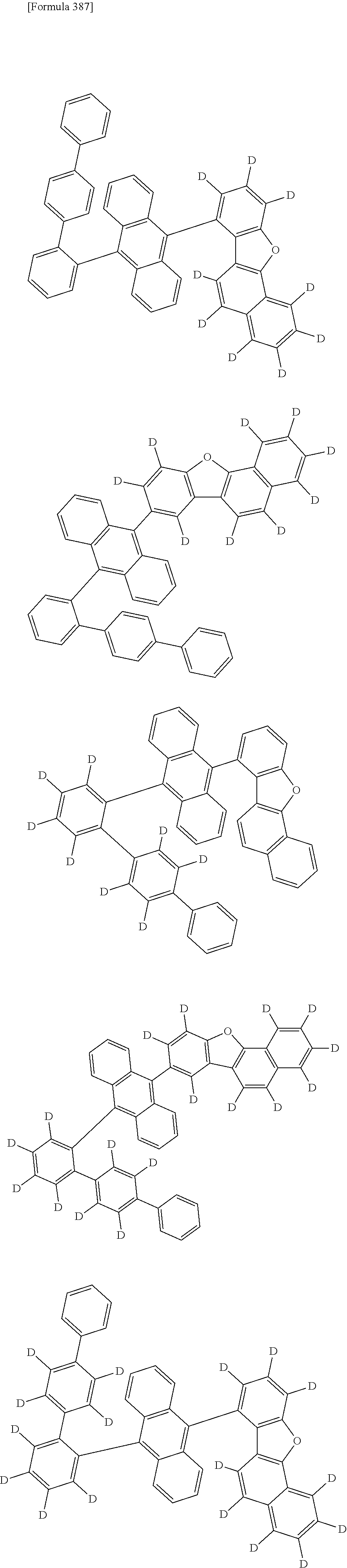

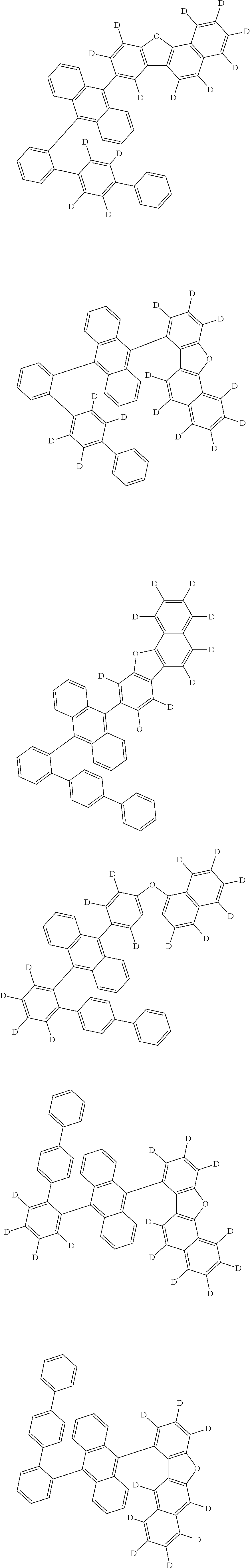

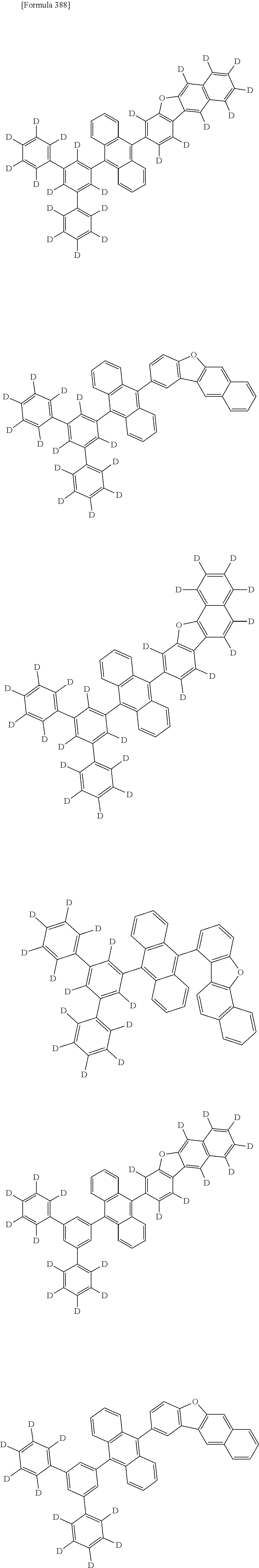

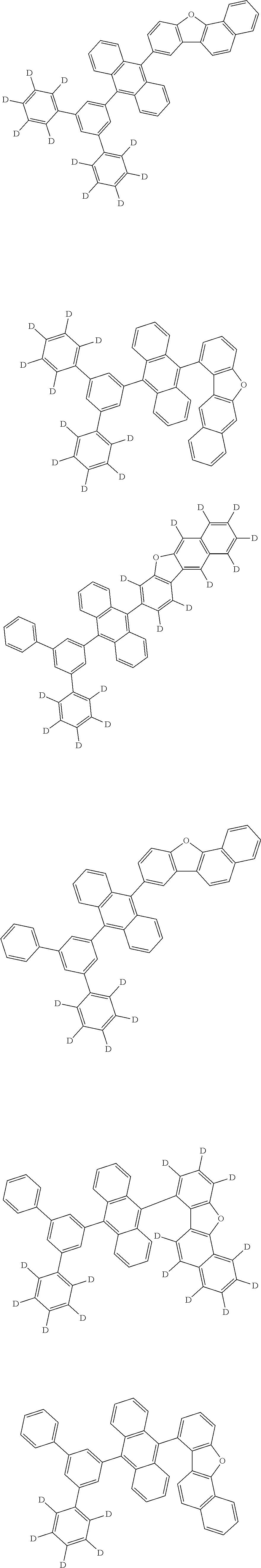

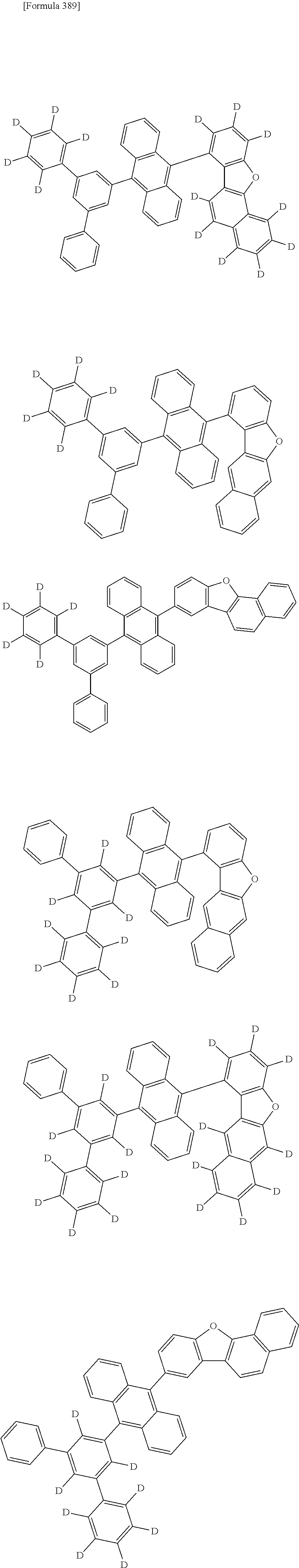

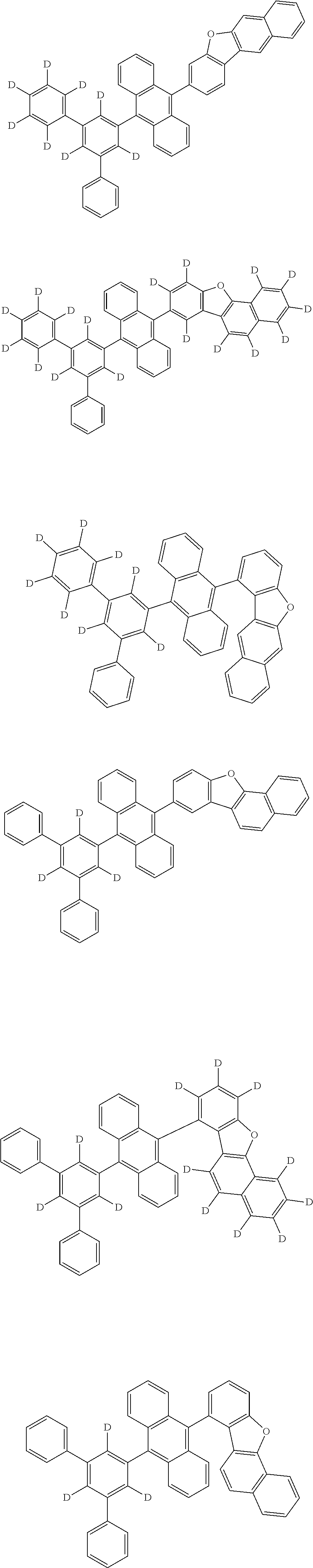

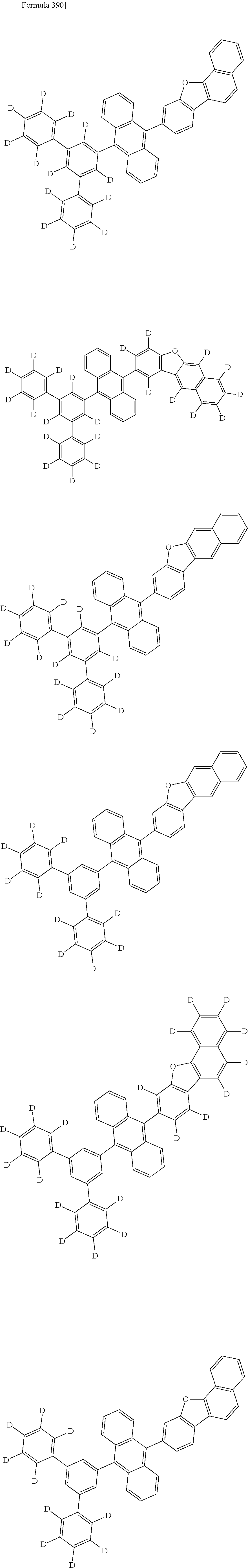

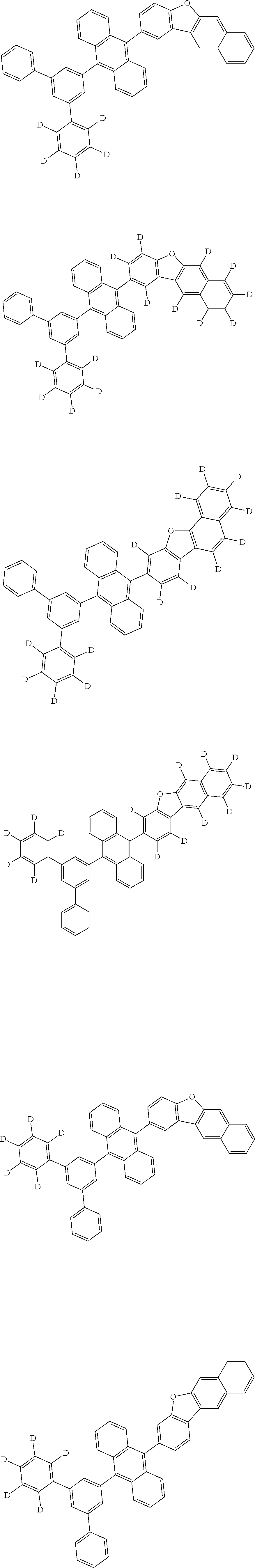

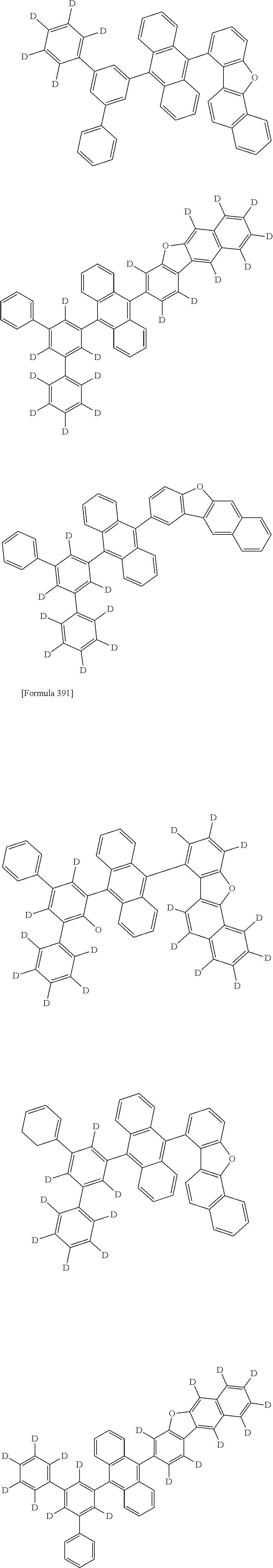

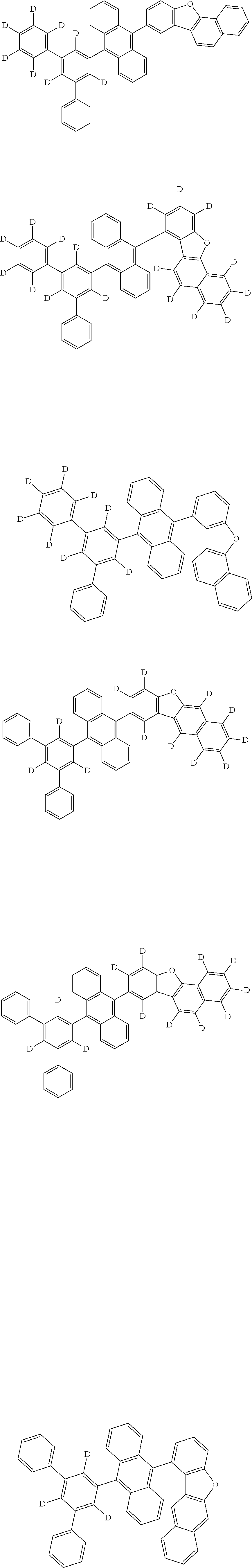

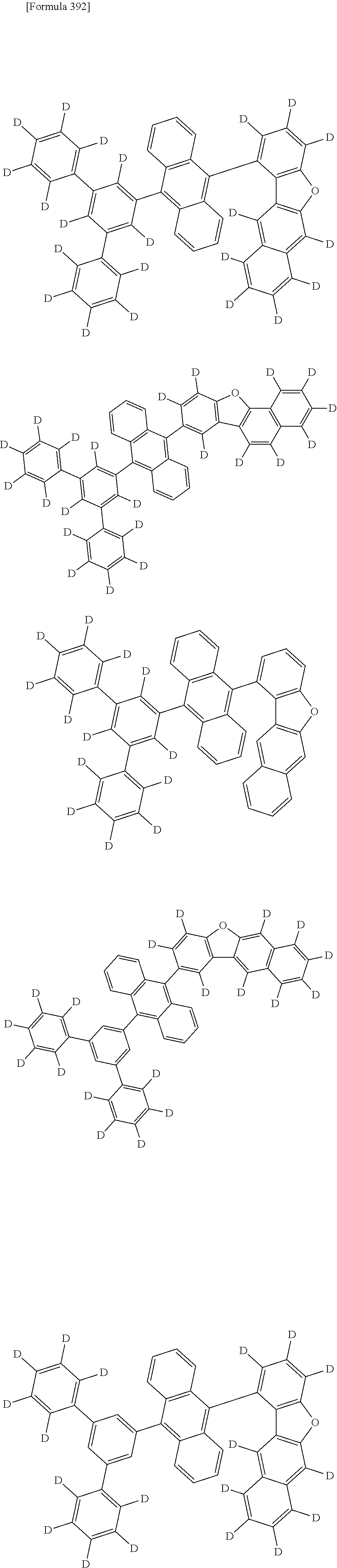

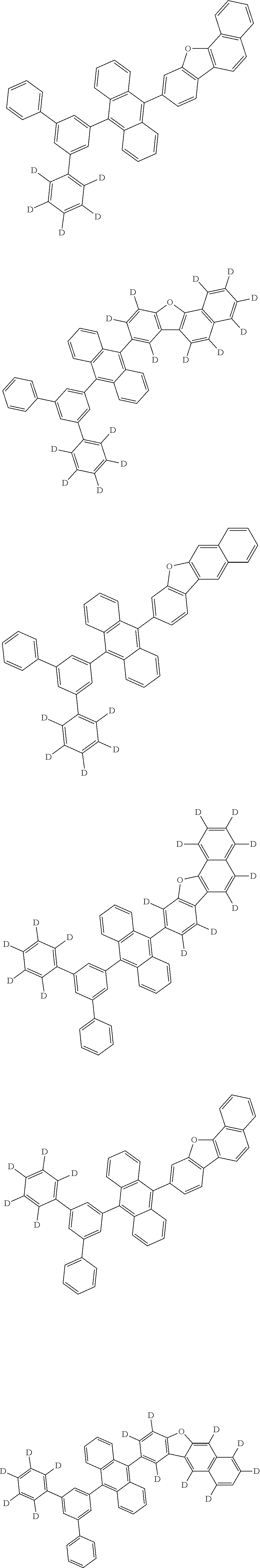

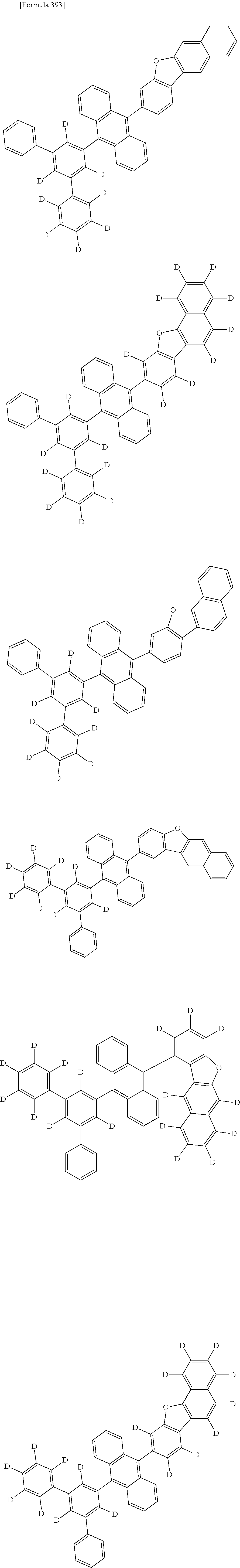

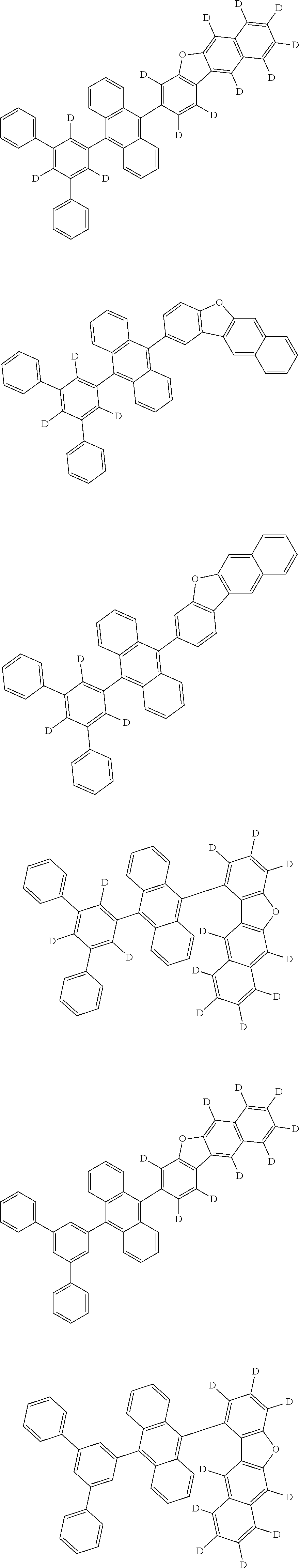

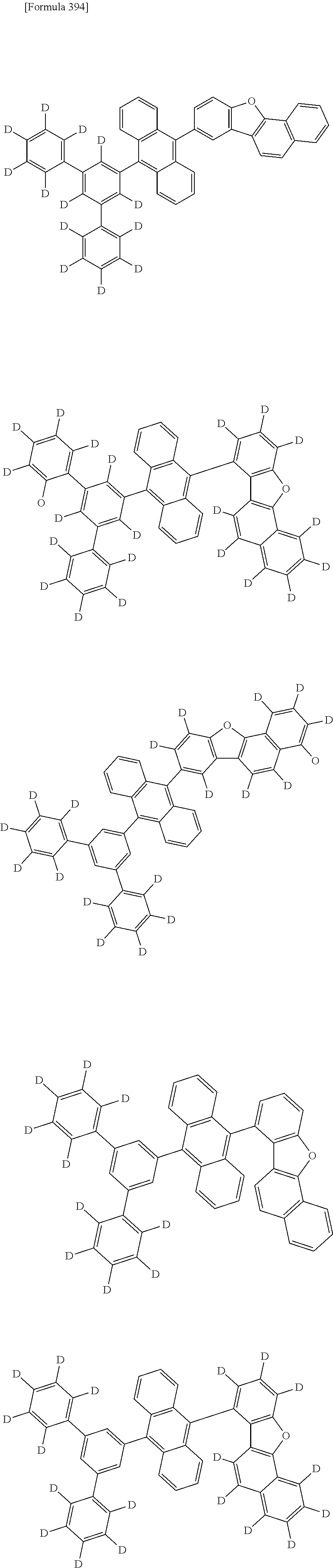

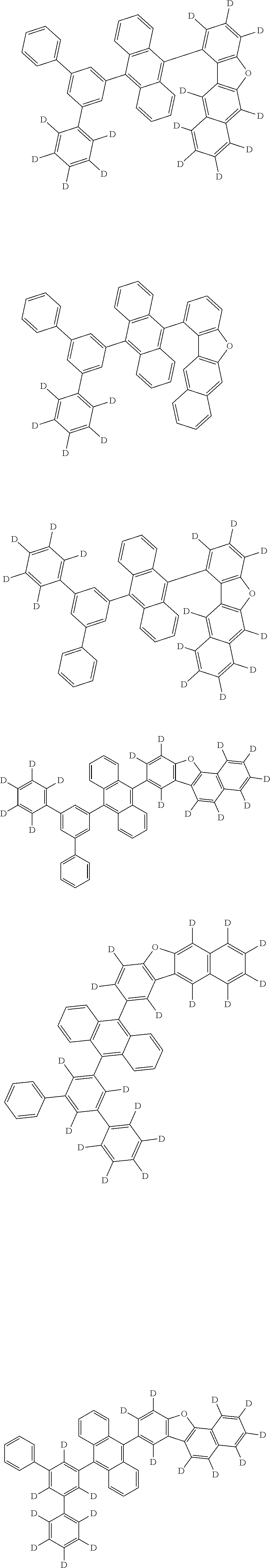

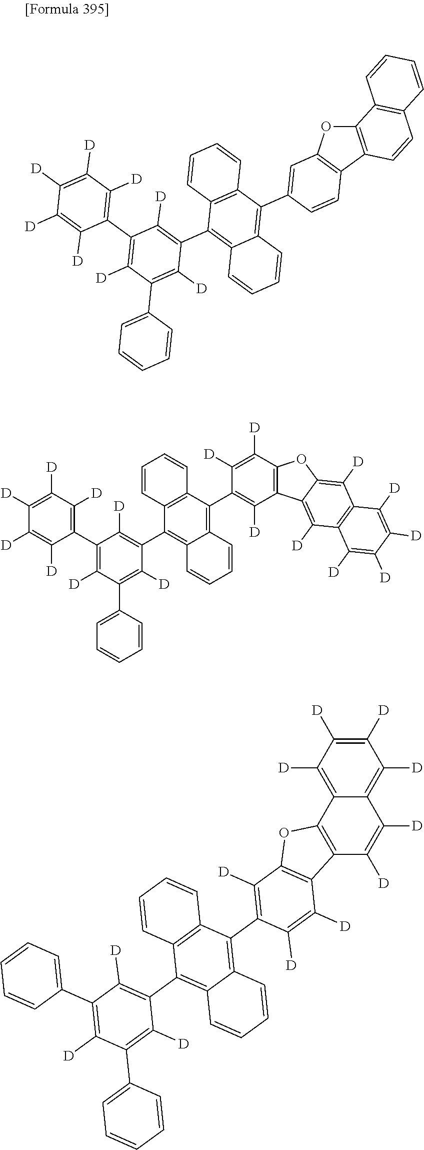 [SMILES: [2H]c1c([2H])c([2H])c(-c2c([2H])c(-c3ccccc3)c([2H])c(-c3c4ccccc4c(-c4ccc5c(c4)oc4c6ccccc6ccc54)c4ccccc34)c2[2H])c([2H])c1[2H] ; [2H]c1c([2H])c([2H])c(-c2c([2H])c(-c3ccccc3)c([2H])c(-c3c4ccccc4c(-c4c([2H])c([2H])c5oc6c([2H])c7c([2H])c([2H])c([2H])c([2H])c7c([2H])c6c5c4[2H])c4ccccc34)c2[2H])c([2H])c1[2H] ; [2H]c1c(-c2ccccc2)c([2H])c(-c2c3ccccc3c(-c3c([2H])c([2H])c4c(oc5c6c([2H])c([2H])c([2H])c([2H])c6c([2H])c([2H])c54)c3[2H])c3ccccc23)c([2H])c1-c1ccccc1]

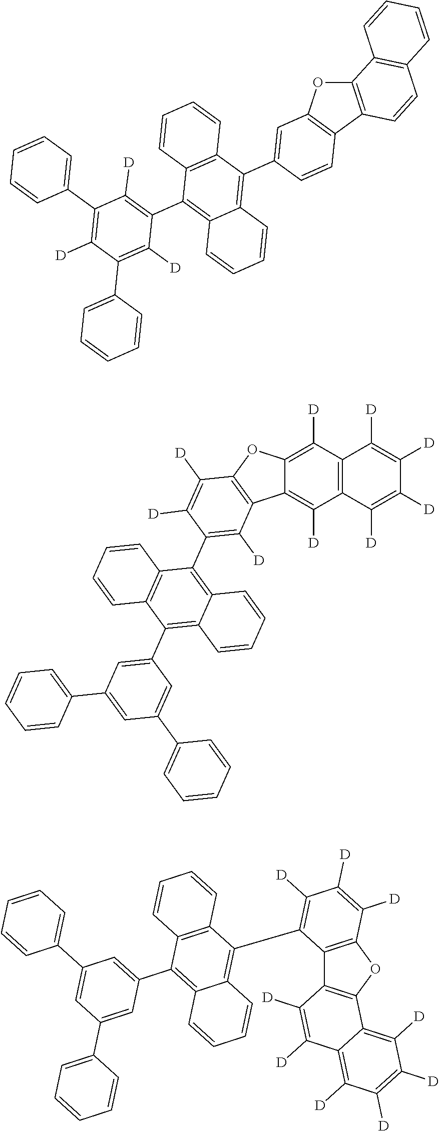 [SMILES: [2H]c1c(-c2ccccc2)c([2H])c(-c2c3ccccc3c(-c3ccc4c(c3)oc3c5ccccc5ccc43)c3ccccc23)c([2H])c1-c1ccccc1 ; [2H]c1c(-c2c3ccccc3c(-c3cc(-c4ccccc4)cc(-c4ccccc4)c3)c3ccccc23)c([2H])c2c(oc3c([2H])c4c([2H])c([2H])c([2H])c([2H])c4c([2H])c32)c1[2H] ; [2H]c1c([2H])c(-c2c3ccccc3c(-c3cc(-c4ccccc4)cc(-c4ccccc4)c3)c3ccccc23)c2c(oc3c4c([2H])c([2H])c([2H])c([2H])c4c([2H])c([2H])c32)c1[2H]]

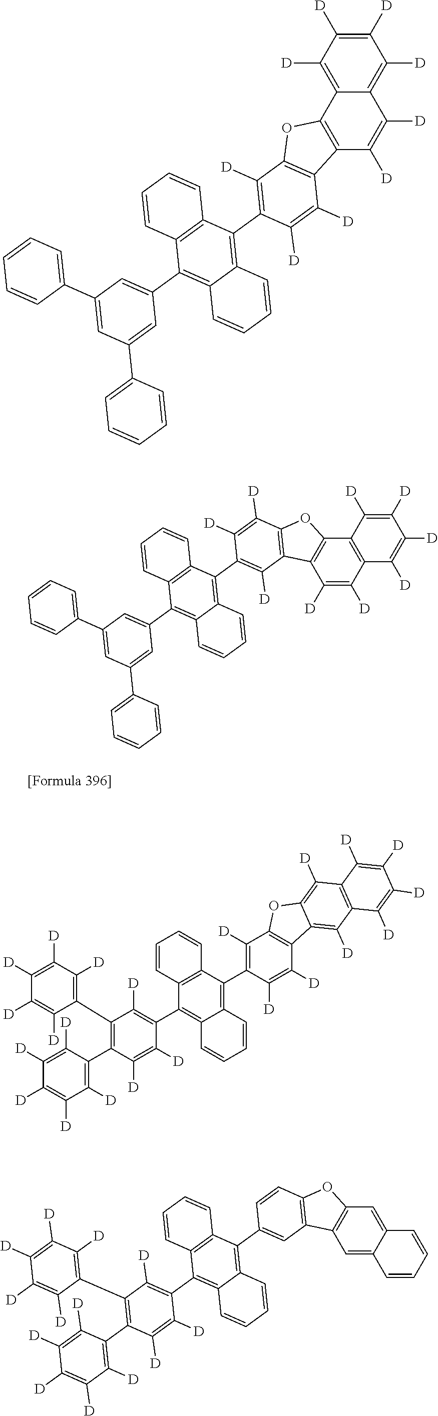

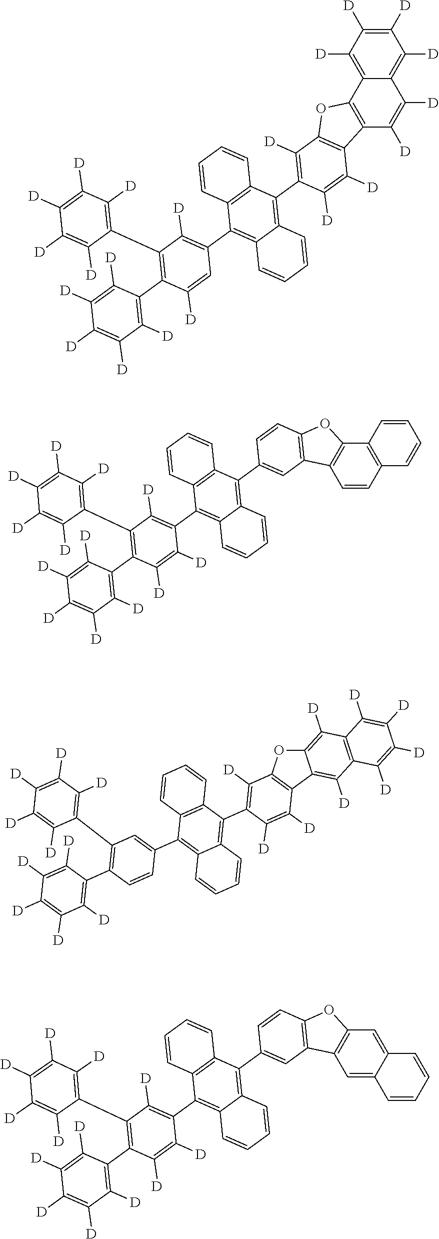

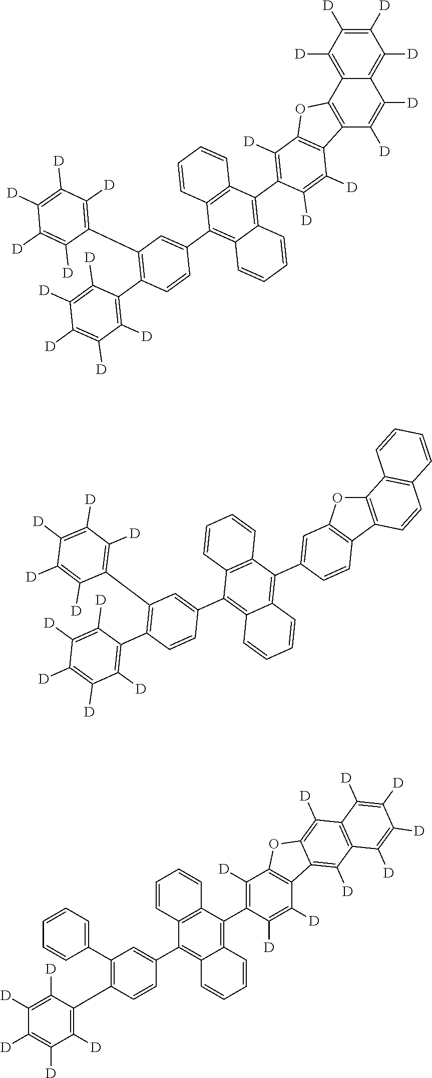 [SMILES: [2H]c1c([2H])c([2H])c(-c2ccc(-c3c4ccccc4c(-c4c([2H])c([2H])c5c(oc6c7c([2H])c([2H])c([2H])c([2H])c7c([2H])c([2H])c65)c4[2H])c4ccccc34)cc2-c2c([2H])c([2H])c([2H])c([2H])c2[2H])c([2H])c1[2H] ; [2H]c1c([2H])c([2H])c(-c2ccc(-c3c4ccccc4c(-c4ccc5c(c4)oc4c6ccccc6ccc54)c4ccccc34)cc2-c2c([2H])c([2H])c([2H])c([2H])c2[2H])c([2H])c1[2H] ; [2H]c1c([2H])c([2H])c(-c2ccc(-c3c4ccccc4c(-c4c([2H])c([2H])c5c(oc6c([2H])c7c([2H])c([2H])c([2H])c([2H])c7c([2H])c65)c4[2H])c4ccccc34)cc2-c2ccccc2)c([2H])c1[2H]]

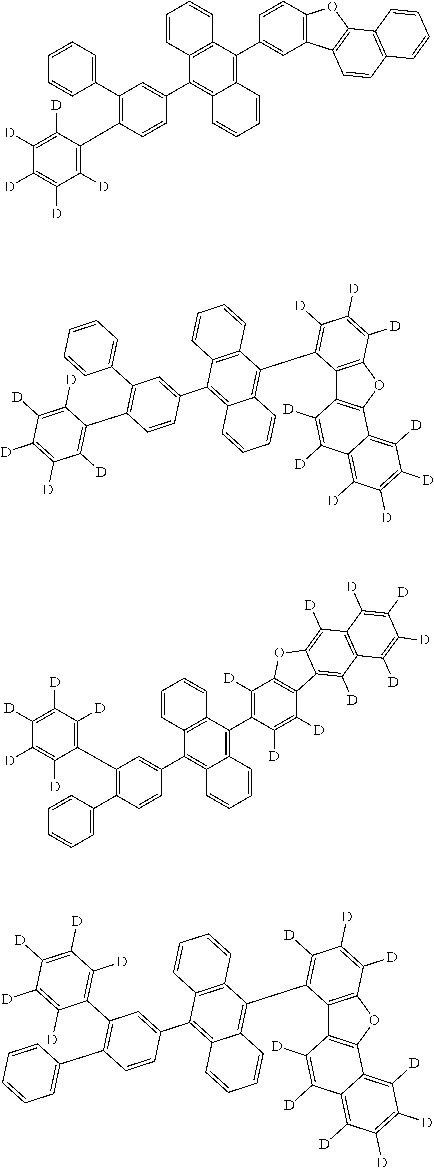

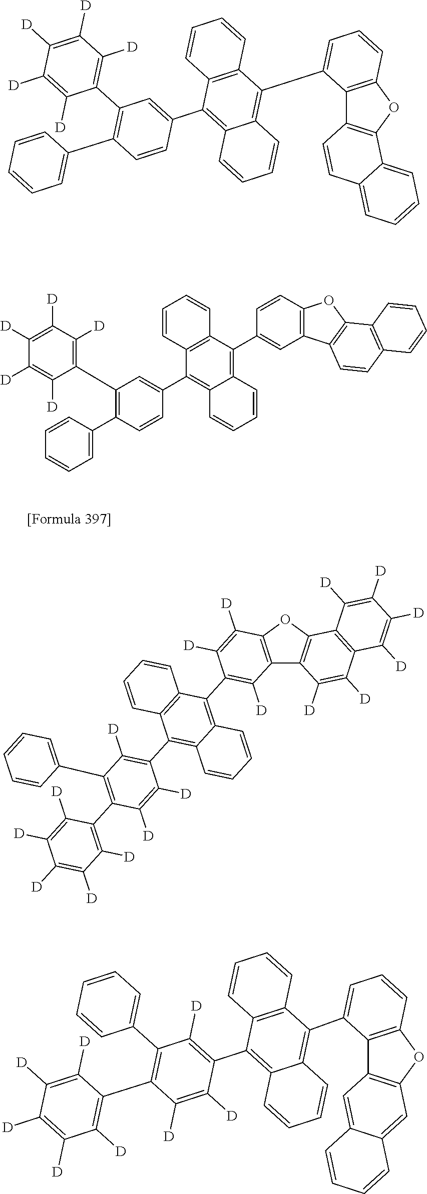

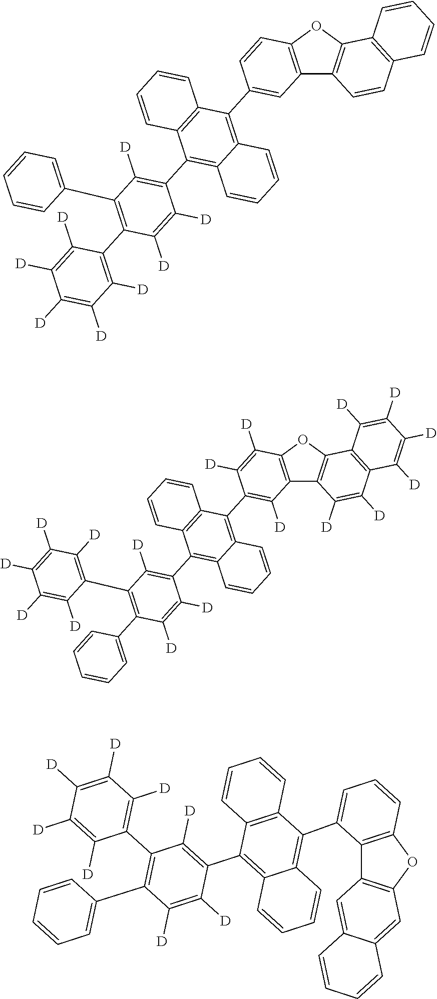 [SMILES: [2H]c1c([2H])c([2H])c(-c2c([2H])c([2H])c(-c3c4ccccc4c(-c4ccc5oc6c7ccccc7ccc6c5c4)c4ccccc34)c([2H])c2-c2ccccc2)c([2H])c1[2H] ; [2H]c1c([2H])c([2H])c(-c2c([2H])c(-c3c4ccccc4c(-c4c([2H])c([2H])c5oc6c7c([2H])c([2H])c([2H])c([2H])c7c([2H])c([2H])c6c5c4[2H])c4ccccc34)c([2H])c([2H])c2-c2ccccc2)c([2H])c1[2H] ; [2H]c1c([2H])c([2H])c(-c2c([2H])c(-c3c4ccccc4c(-c4cccc5oc6cc7ccccc7cc6c45)c4ccccc34)c([2H])c([2H])c2-c2ccccc2)c([2H])c1[2H]]

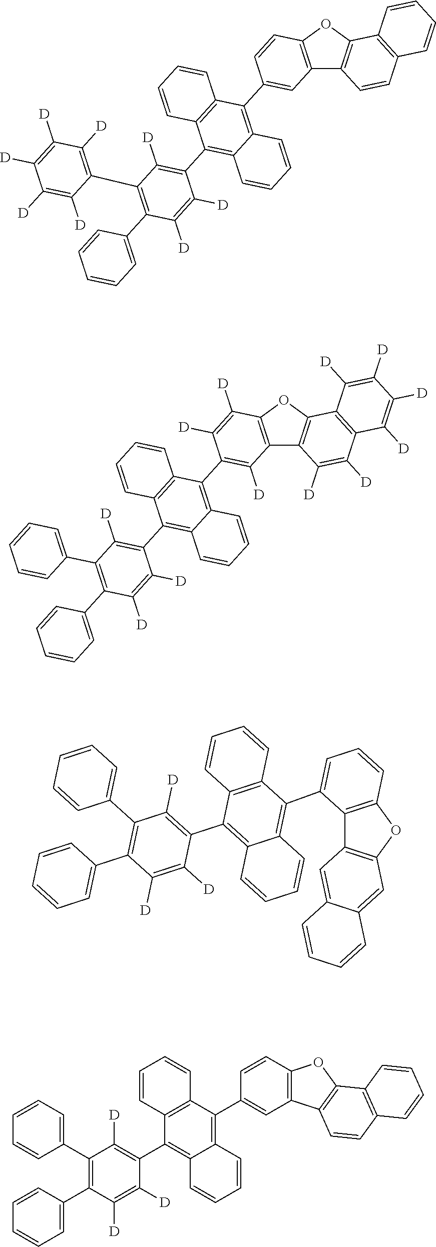 [SMILES: [2H]c1c([2H])c([2H])c(-c2c([2H])c(-c3c4ccccc4c(-c4ccc5oc6c7ccccc7ccc6c5c4)c4ccccc34)c([2H])c([2H])c2-c2ccccc2)c([2H])c1[2H] ; [2H]c1c([2H])c(-c2ccccc2)c(-c2ccccc2)c([2H])c1-c1c2ccccc2c(-c2c([2H])c([2H])c3oc4c5c([2H])c([2H])c([2H])c([2H])c5c([2H])c([2H])c4c3c2[2H])c2ccccc12 ; [2H]c1c([2H])c(-c2ccccc2)c(-c2ccccc2)c([2H])c1-c1c2ccccc2c(-c2cccc3oc4cc5ccccc5cc4c23)c2ccccc12 ; [2H]c1c([2H])c(-c2ccccc2)c(-c2ccccc2)c([2H])c1-c1c2ccccc2c(-c2ccc3oc4c5ccccc5ccc4c3c2)c2ccccc12]

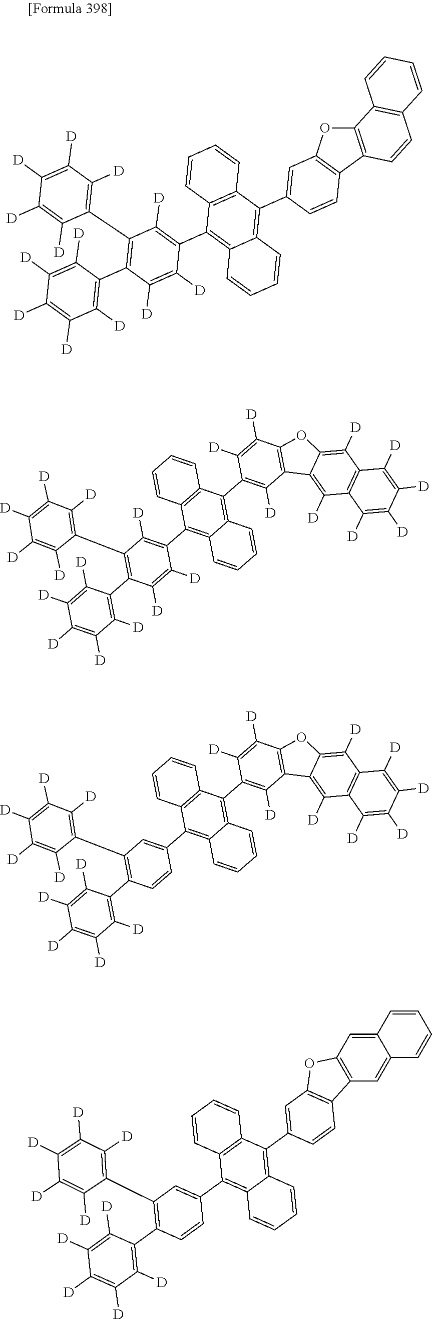

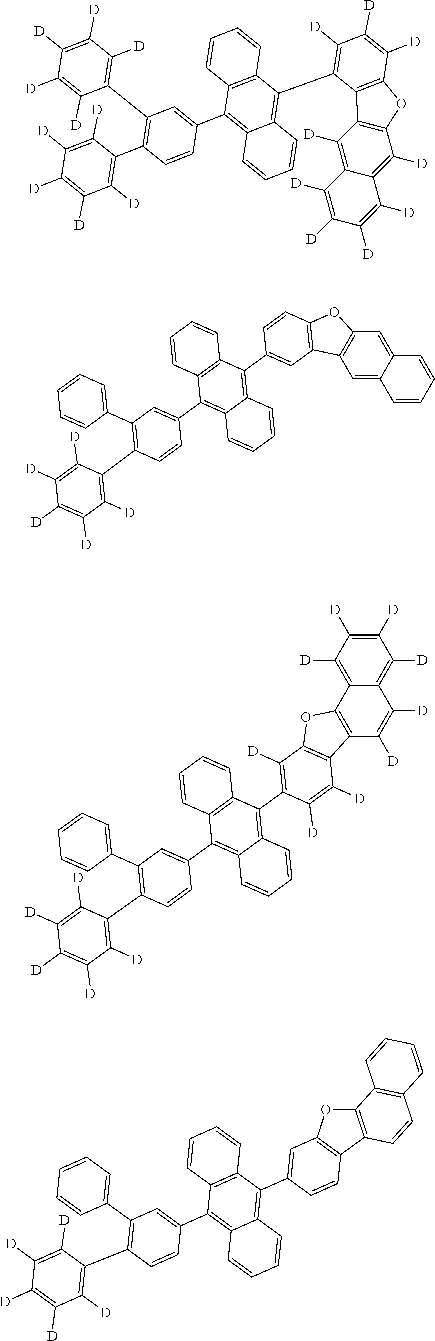

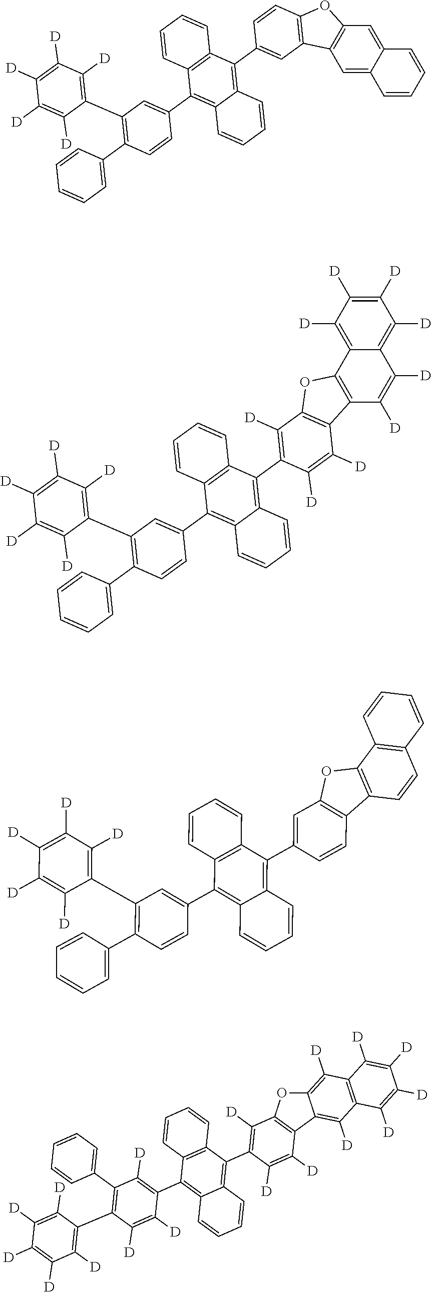

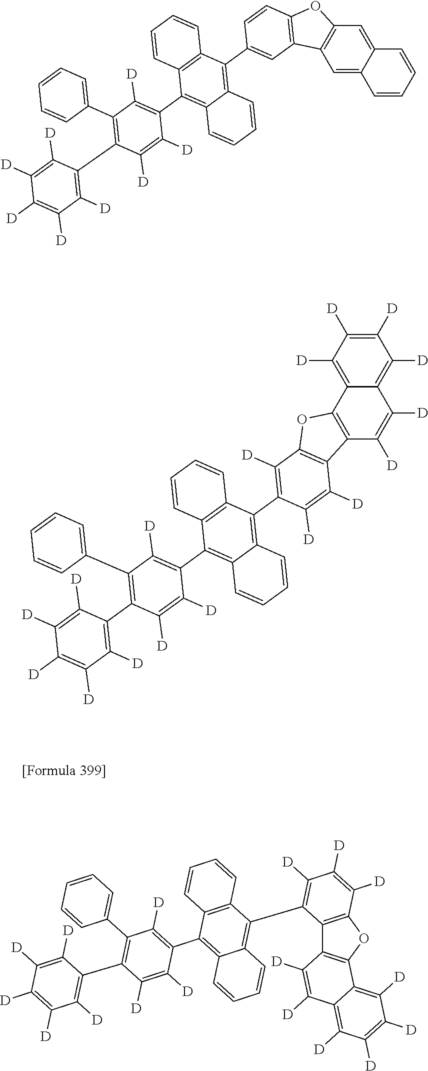 [SMILES: [2H]c1c([2H])c([2H])c(-c2c([2H])c([2H])c(-c3c4ccccc4c(-c4ccc5oc6cc7ccccc7cc6c5c4)c4ccccc34)c([2H])c2-c2ccccc2)c([2H])c1[2H] ; [2H]c1c([2H])c([2H])c(-c2c([2H])c([2H])c(-c3c4ccccc4c(-c4c([2H])c([2H])c5c(oc6c7c([2H])c([2H])c([2H])c([2H])c7c([2H])c([2H])c65)c4[2H])c4ccccc34)c([2H])c2-c2ccccc2)c([2H])c1[2H] ; [2H]c1c([2H])c([2H])c(-c2c([2H])c([2H])c(-c3c4ccccc4c(-c4c([2H])c([2H])c([2H])c5oc6c7c([2H])c([2H])c([2H])c([2H])c7c([2H])c([2H])c6c45)c4ccccc34)c([2H])c2-c2ccccc2)c([2H])c1[2H]]

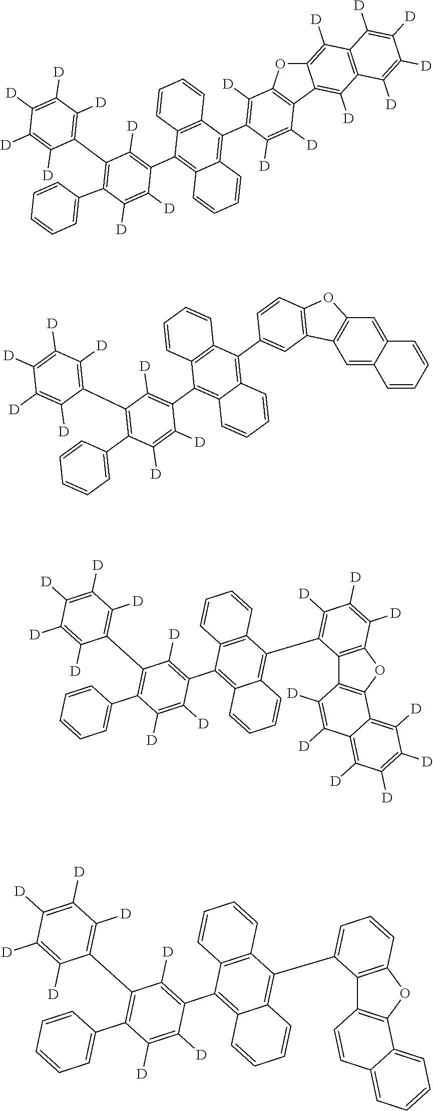

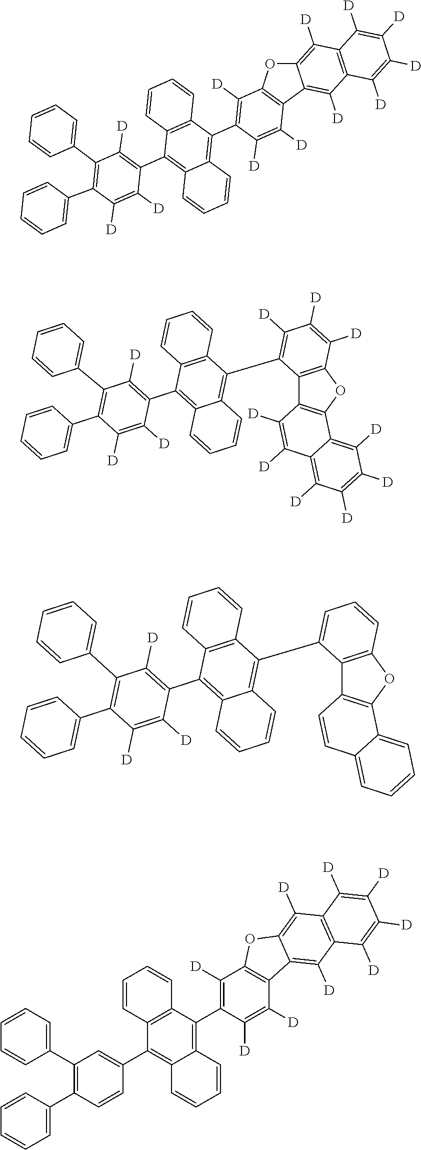

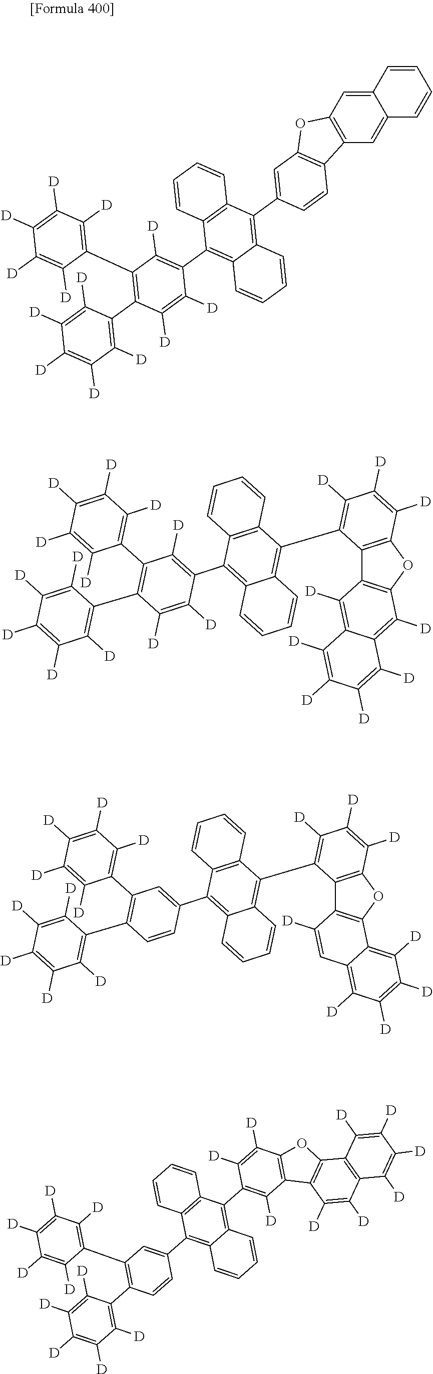

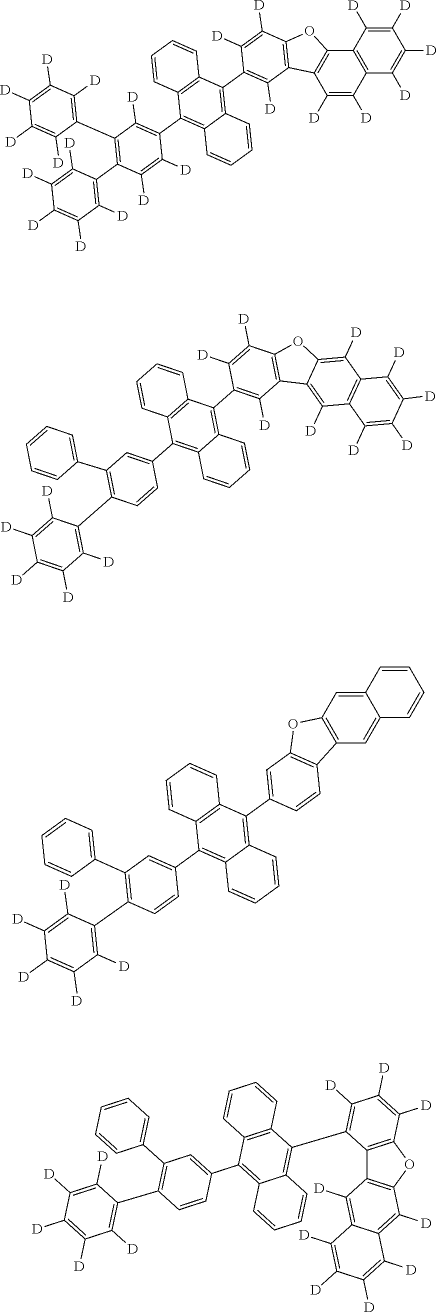

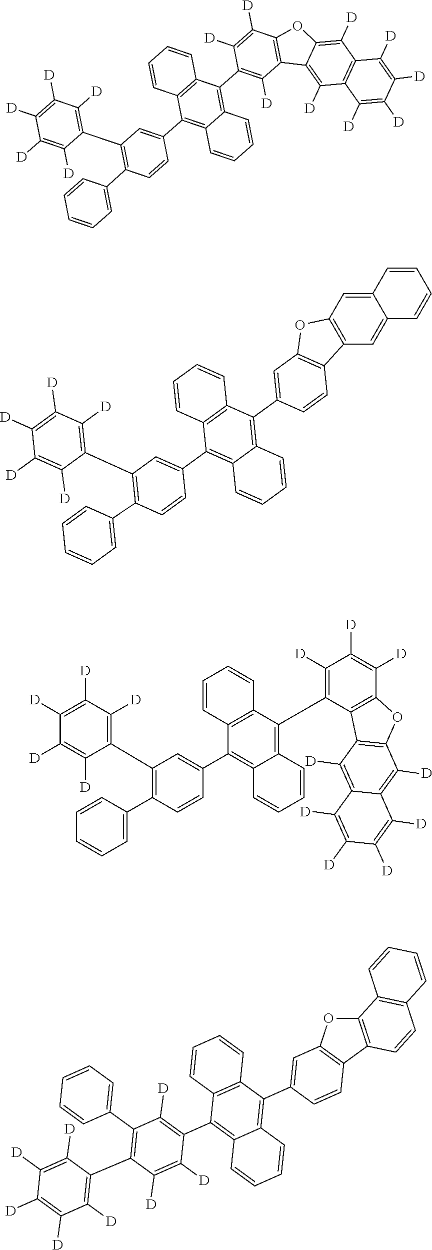

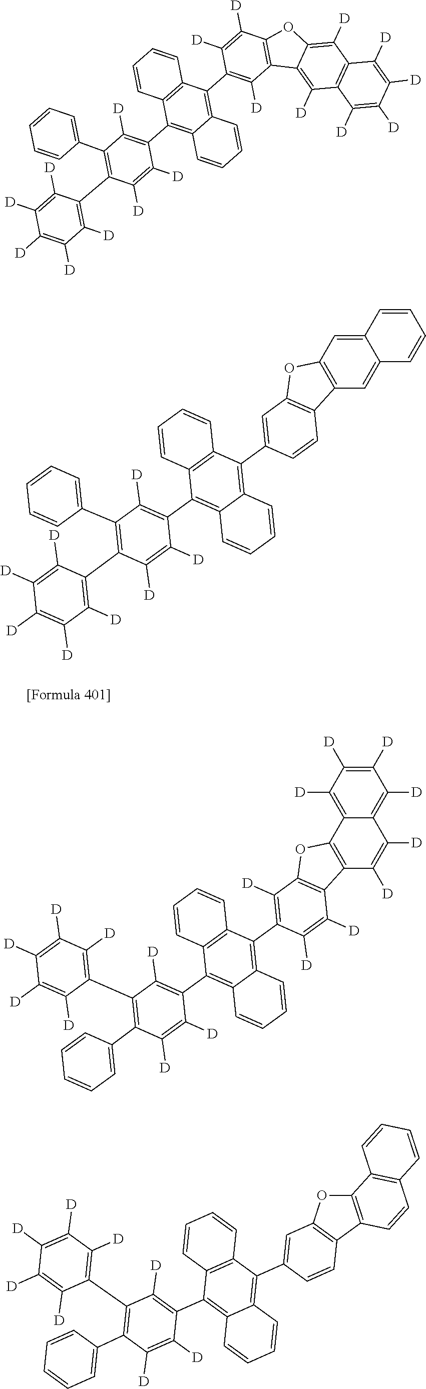

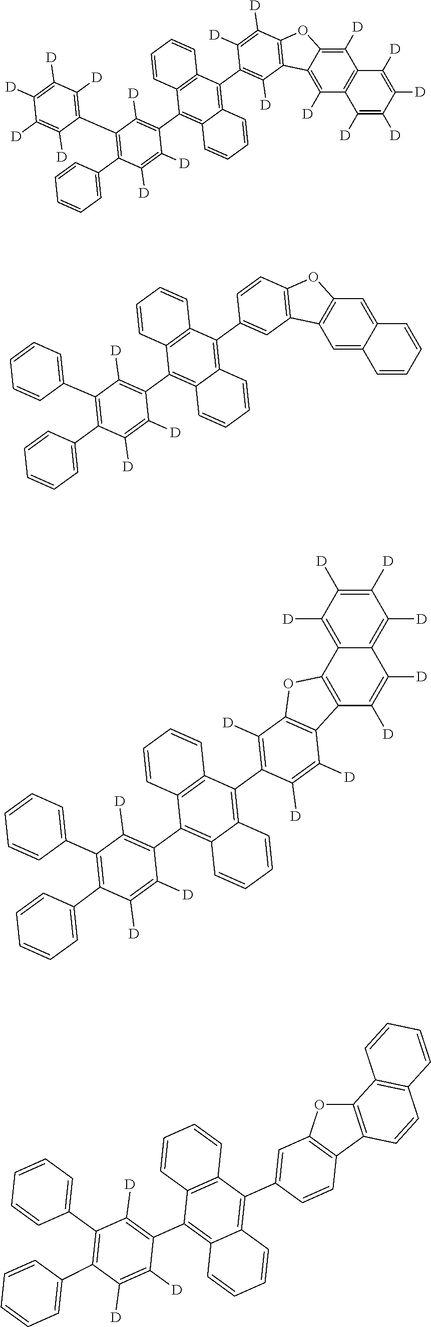

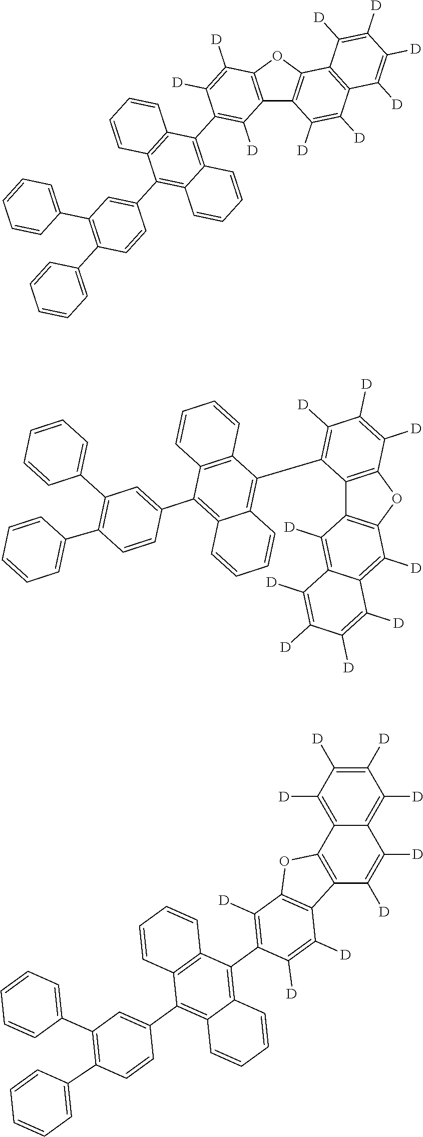 [SMILES: [2H]c1c(-c2c3ccccc3c(-c3ccc(-c4ccccc4)c(-c4ccccc4)c3)c3ccccc23)c([2H])c2c(oc3c4c([2H])c([2H])c([2H])c([2H])c4c([2H])c([2H])c32)c1[2H] ; [2H]c1c([2H])c(-c2c3ccccc3c(-c3ccc(-c4ccccc4)c(-c4ccccc4)c3)c3ccccc23)c2c(oc3c([2H])c4c([2H])c([2H])c([2H])c([2H])c4c([2H])c32)c1[2H] ; [2H]c1c([2H])c([2H])c2c(c1[2H])c([2H])c([2H])c1c2oc2c([2H])c(-c3c4ccccc4c(-c4ccc(-c5ccccc5)c(-c5ccccc5)c4)c4ccccc34)c([2H])c([2H])c21]

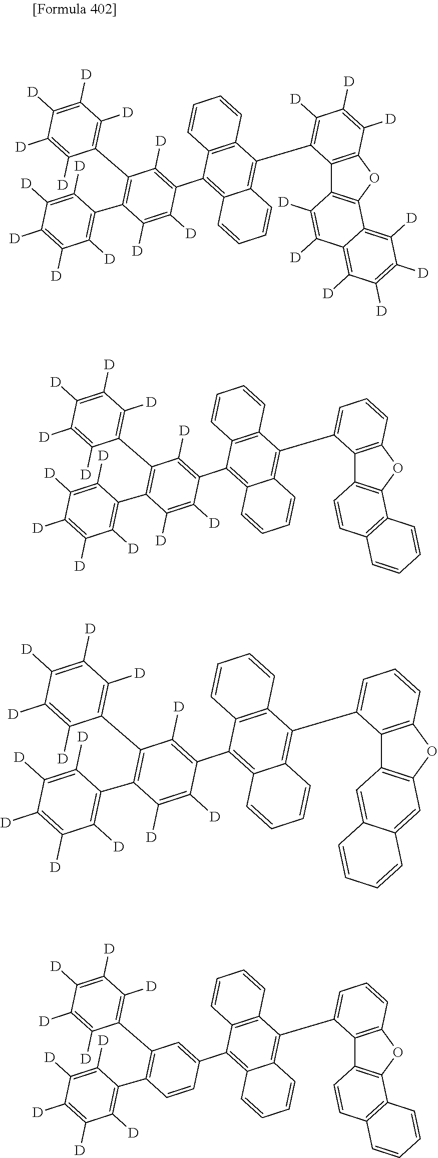

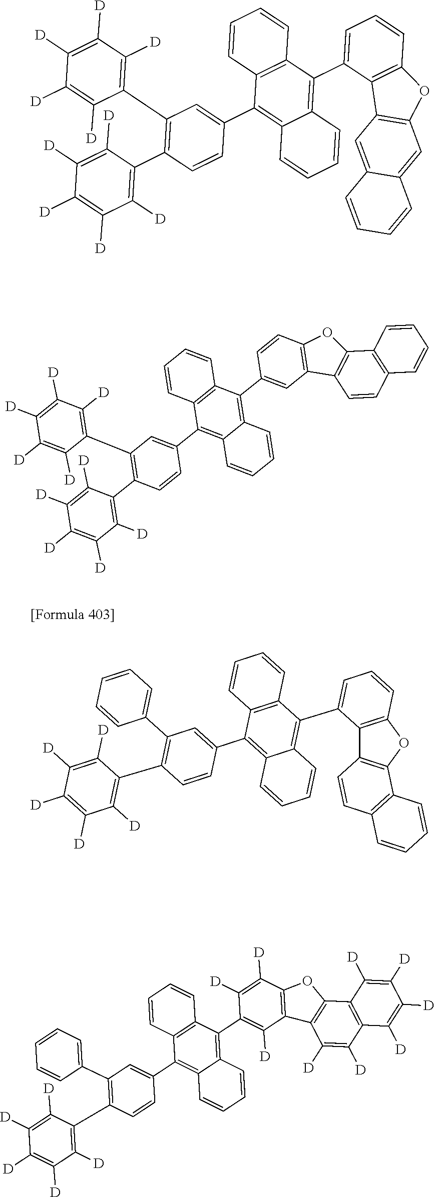

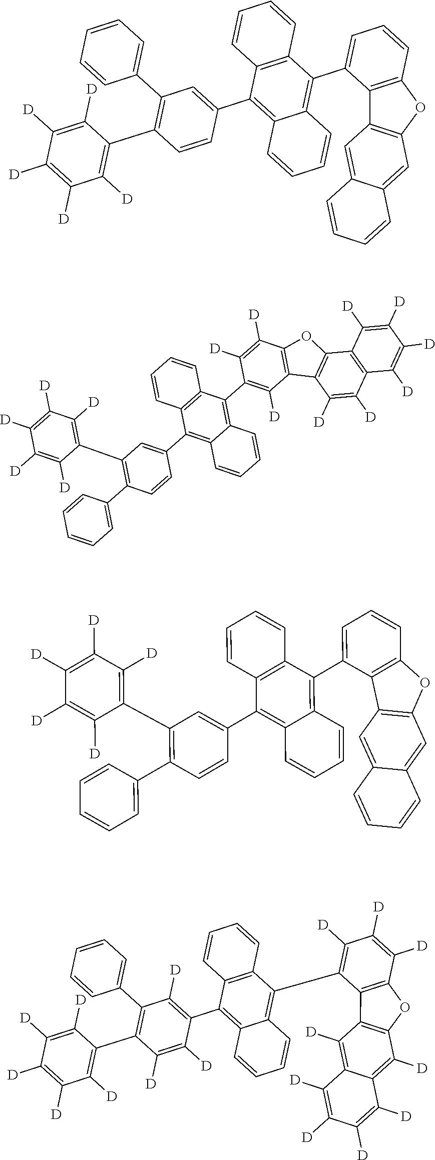

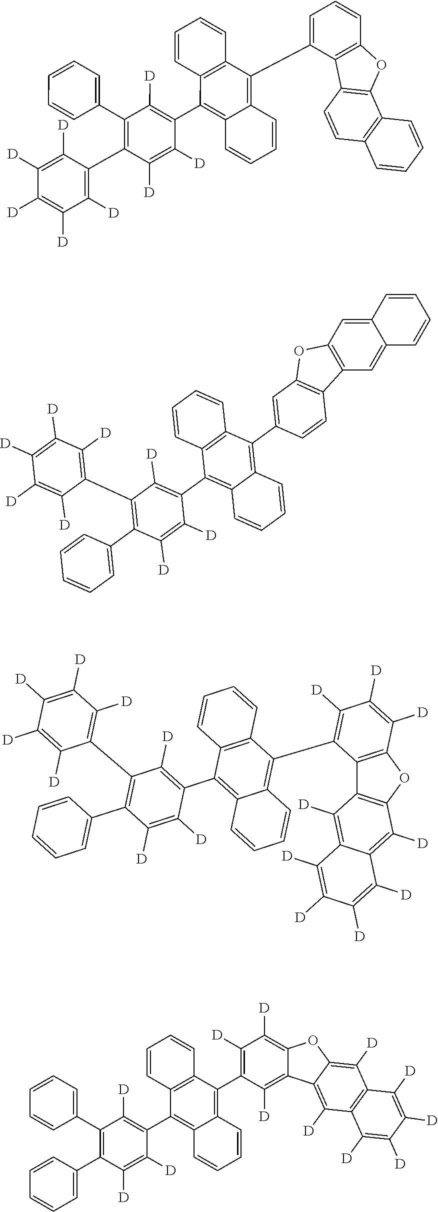

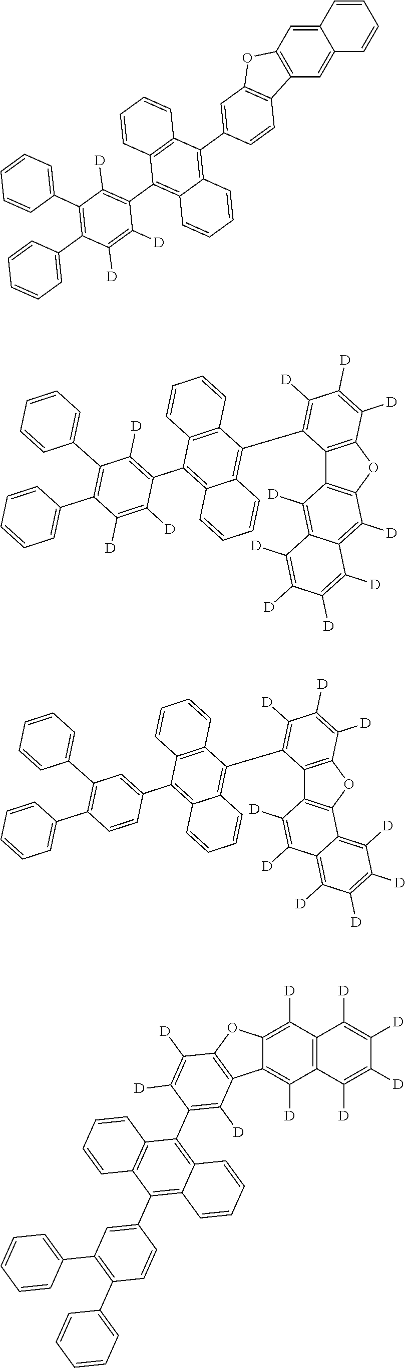

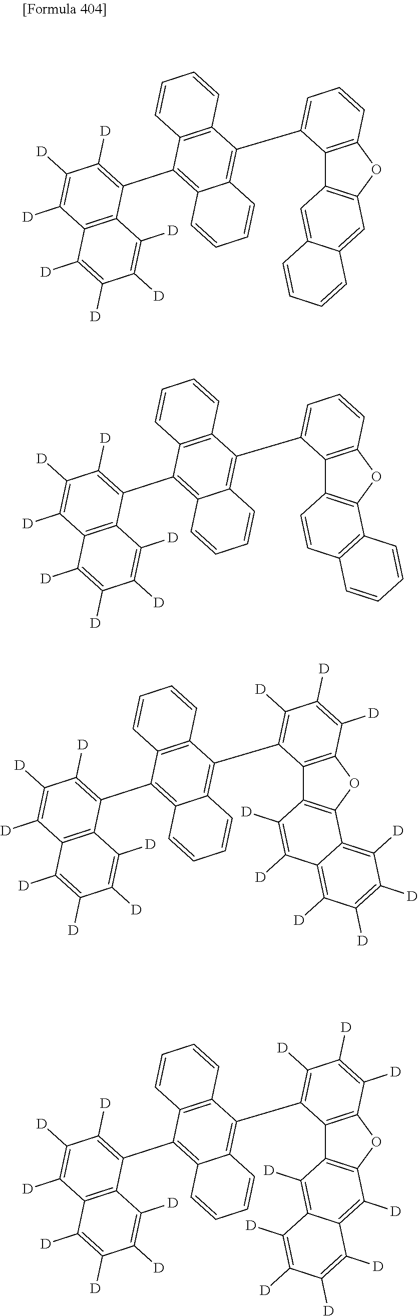

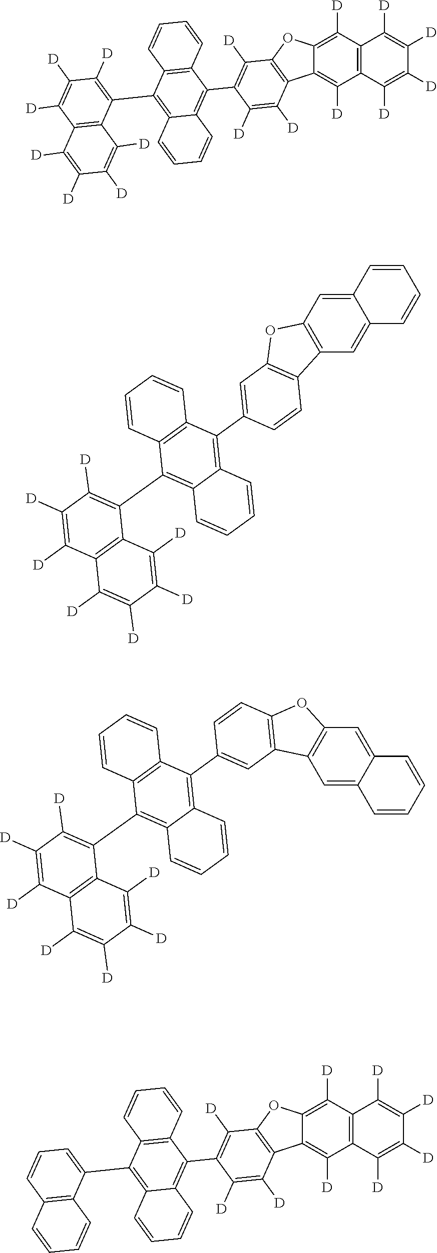 [SMILES: [2H]c1c([2H])c([2H])c2c(-c3c4ccccc4c(-c4c([2H])c([2H])c5c(oc6c([2H])c7c([2H])c([2H])c([2H])c([2H])c7c([2H])c65)c4[2H])c4ccccc34)c([2H])c([2H])c([2H])c2c1[2H] ; [2H]c1c([2H])c([2H])c2c(-c3c4ccccc4c(-c4ccc5c(c4)oc4cc6ccccc6cc45)c4ccccc34)c([2H])c([2H])c([2H])c2c1[2H] ; [2H]c1c([2H])c([2H])c2c(-c3c4ccccc4c(-c4ccc5oc6cc7ccccc7cc6c5c4)c4ccccc34)c([2H])c([2H])c([2H])c2c1[2H] ; [2H]c1c([2H])c([2H])c2c([2H])c3c(oc4c([2H])c(-c5c6ccccc6c(-c6cccc7ccccc67)c6ccccc56)c([2H])c([2H])c43)c([2H])c2c1[2H]]

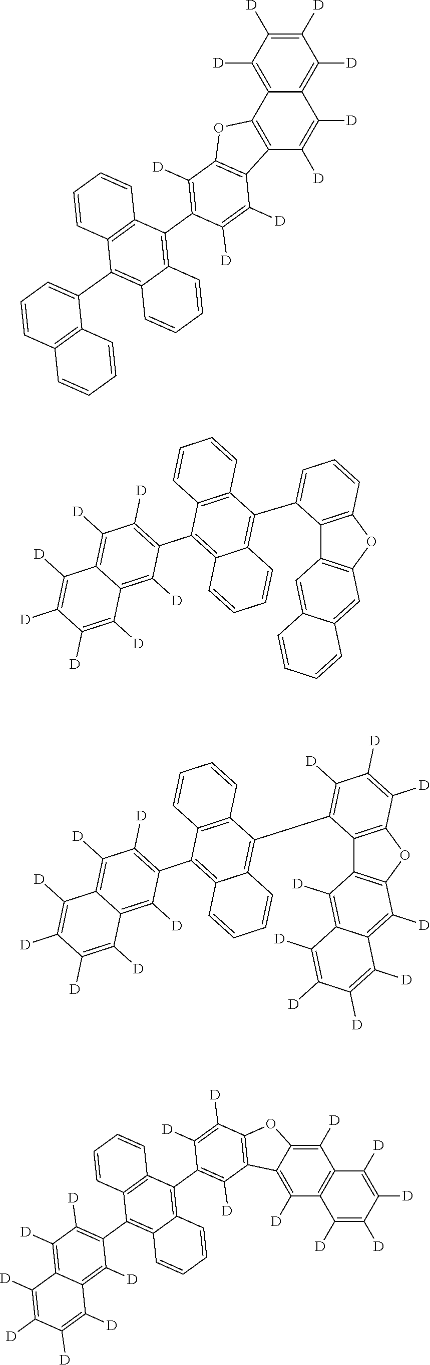

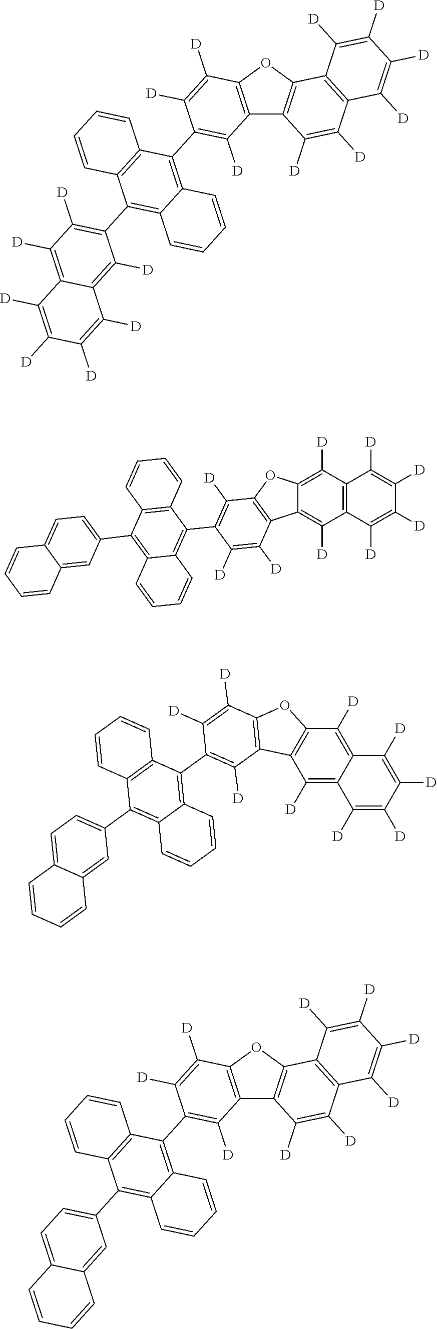

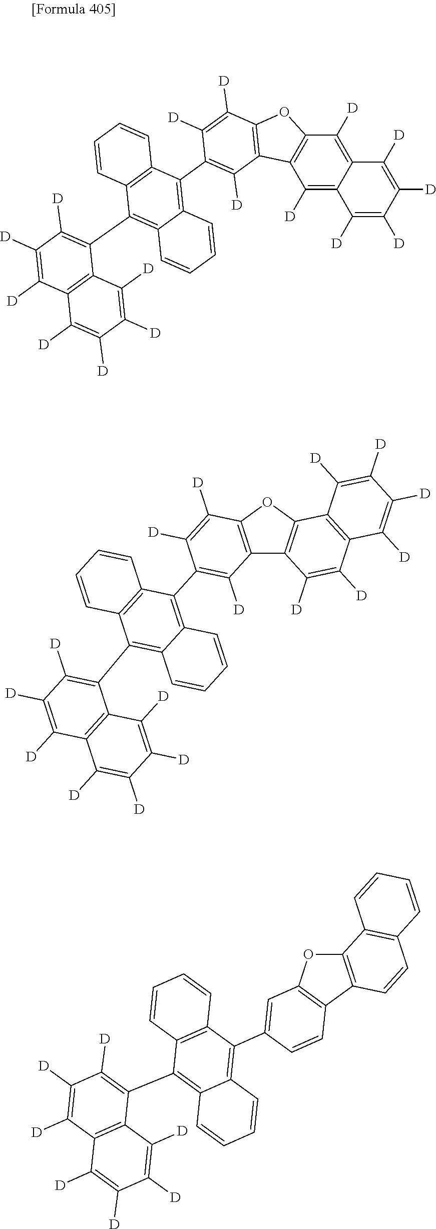 [SMILES: [2H]c1c(-c2c3ccccc3c(-c3c([2H])c([2H])c([2H])c4c([2H])c([2H])c([2H])c([2H])c34)c3ccccc23)c([2H])c2c(oc3c([2H])c4c([2H])c([2H])c([2H])c([2H])c4c([2H])c32)c1[2H] ; [2H]c1c(-c2c3ccccc3c(-c3c([2H])c([2H])c([2H])c4c([2H])c([2H])c([2H])c([2H])c34)c3ccccc23)c([2H])c2c(oc3c4c([2H])c([2H])c([2H])c([2H])c4c([2H])c([2H])c32)c1[2H] ; [2H]c1c([2H])c([2H])c2c(-c3c4ccccc4c(-c4ccc5c(c4)oc4c6ccccc6ccc54)c4ccccc34)c([2H])c([2H])c([2H])c2c1[2H]]

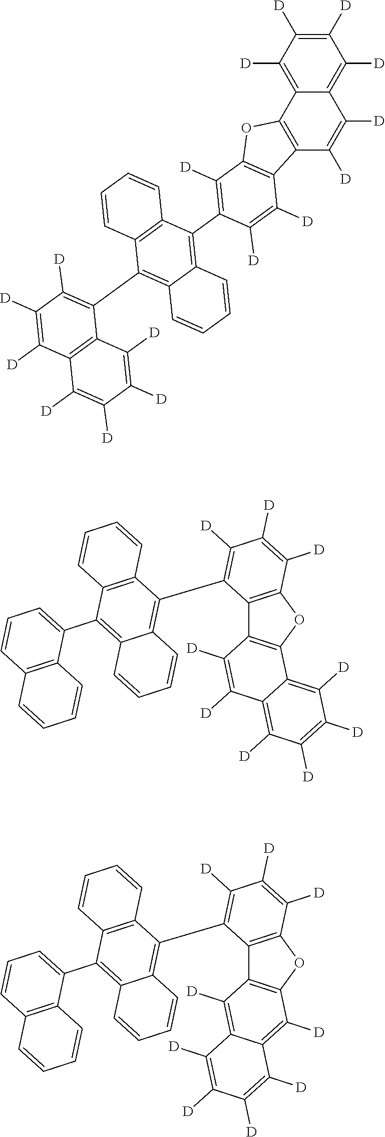 [SMILES: [2H]c1c([2H])c([2H])c2c(-c3c4ccccc4c(-c4c([2H])c([2H])c5c(oc6c7c([2H])c([2H])c([2H])c([2H])c7c([2H])c([2H])c65)c4[2H])c4ccccc34)c([2H])c([2H])c([2H])c2c1[2H] ; [2H]c1c([2H])c(-c2c3ccccc3c(-c3cccc4ccccc34)c3ccccc23)c2c(oc3c4c([2H])c([2H])c([2H])c([2H])c4c([2H])c([2H])c32)c1[2H] ; [2H]c1c([2H])c(-c2c3ccccc3c(-c3cccc4ccccc34)c3ccccc23)c2c(oc3c([2H])c4c([2H])c([2H])c([2H])c([2H])c4c([2H])c32)c1[2H]]

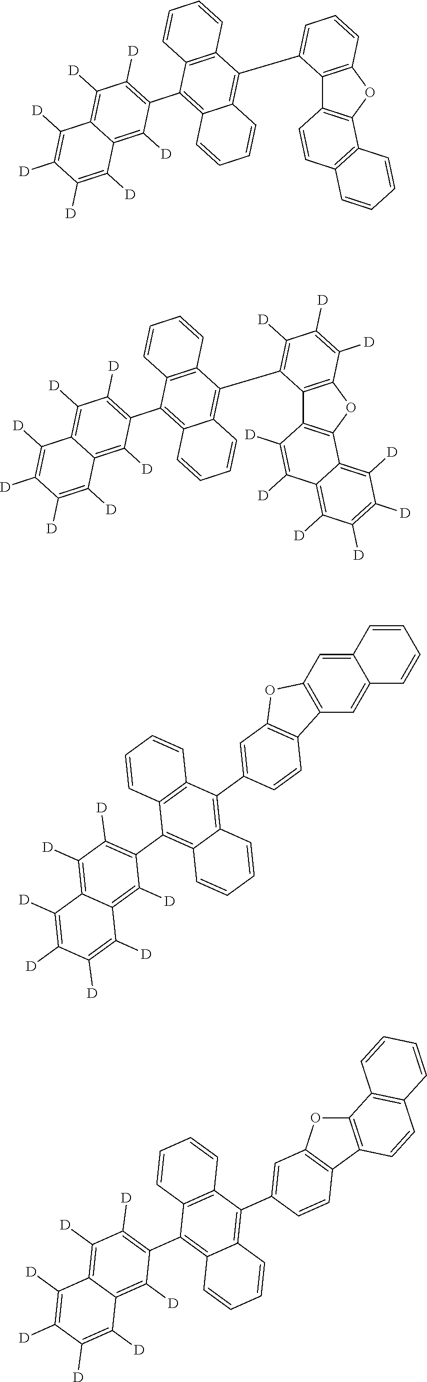 [SMILES: [2H]c1c([2H])c([2H])c2c([2H])c(-c3c4ccccc4c(-c4cccc5oc6c7ccccc7ccc6c45)c4ccccc34)c([2H])c([2H])c2c1[2H] ; [2H]c1c([2H])c(-c2c3ccccc3c(-c3c([2H])c([2H])c4c([2H])c([2H])c([2H])c([2H])c4c3[2H])c3ccccc23)c2c(oc3c4c([2H])c([2H])c([2H])c([2H])c4c([2H])c([2H])c32)c1[2H] ; [2H]c1c([2H])c([2H])c2c([2H])c(-c3c4ccccc4c(-c4ccc5c(c4)oc4cc6ccccc6cc45)c4ccccc34)c([2H])c([2H])c2c1[2H] ; [2H]c1c([2H])c([2H])c2c([2H])c(-c3c4ccccc4c(-c4ccc5c(c4)oc4c6ccccc6ccc54)c4ccccc34)c([2H])c([2H])c2c1[2H]]

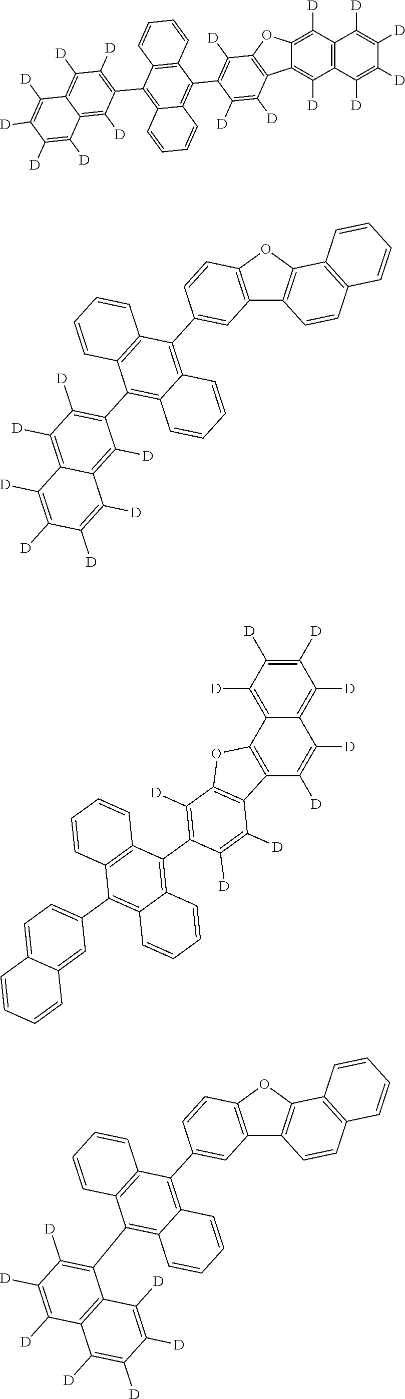 [SMILES: [2H]c1c([2H])c([2H])c2c([2H])c(-c3c4ccccc4c(-c4c([2H])c([2H])c5c(oc6c([2H])c7c([2H])c([2H])c([2H])c([2H])c7c([2H])c65)c4[2H])c4ccccc34)c([2H])c([2H])c2c1[2H] ; [2H]c1c([2H])c([2H])c2c([2H])c(-c3c4ccccc4c(-c4ccc5oc6c7ccccc7ccc6c5c4)c4ccccc34)c([2H])c([2H])c2c1[2H] ; [2H]c1c([2H])c([2H])c2c(c1[2H])c([2H])c([2H])c1c2oc2c([2H])c(-c3c4ccccc4c(-c4ccc5ccccc5c4)c4ccccc34)c([2H])c([2H])c21 ; [2H]c1c([2H])c([2H])c2c(-c3c4ccccc4c(-c4ccc5oc6c7ccccc7ccc6c5c4)c4ccccc34)c([2H])c([2H])c([2H])c2c1[2H]]

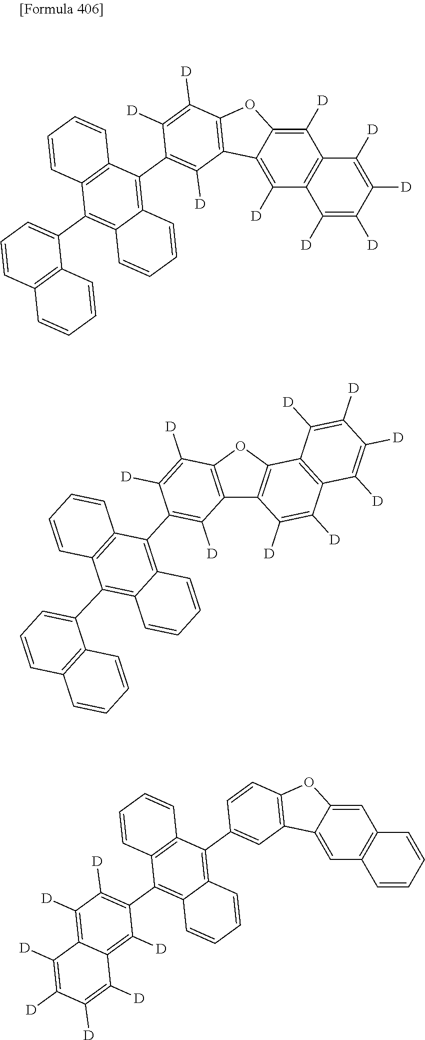 [SMILES: [2H]c1c(-c2c3ccccc3c(-c3cccc4ccccc34)c3ccccc23)c([2H])c2c(oc3c([2H])c4c([2H])c([2H])c([2H])c([2H])c4c([2H])c32)c1[2H] ; [2H]c1c(-c2c3ccccc3c(-c3cccc4ccccc34)c3ccccc23)c([2H])c2c(oc3c4c([2H])c([2H])c([2H])c([2H])c4c([2H])c([2H])c32)c1[2H] ; [2H]c1c([2H])c([2H])c2c([2H])c(-c3c4ccccc4c(-c4ccc5oc6cc7ccccc7cc6c5c4)c4ccccc34)c([2H])c([2H])c2c1[2H]]

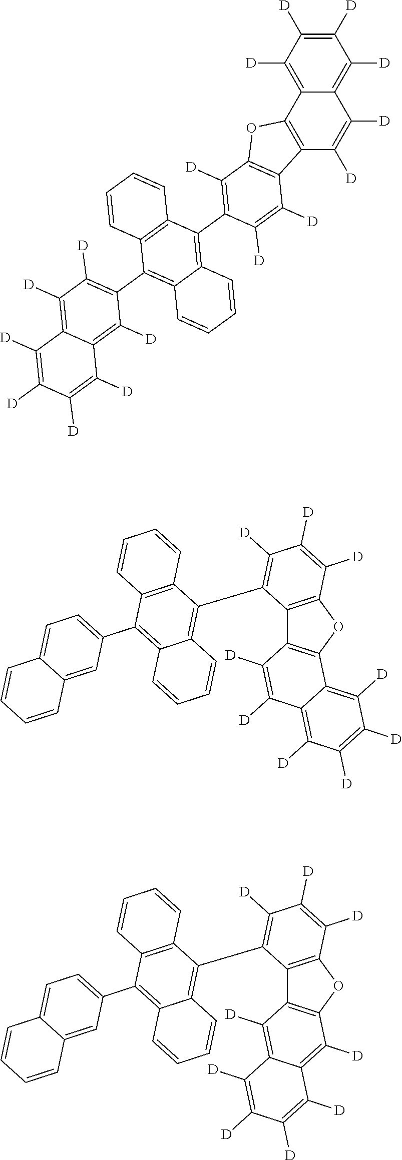 [SMILES: [2H]c1c([2H])c([2H])c2c([2H])c(-c3c4ccccc4c(-c4c([2H])c([2H])c5c(oc6c7c([2H])c([2H])c([2H])c([2H])c7c([2H])c([2H])c65)c4[2H])c4ccccc34)c([2H])c([2H])c2c1[2H] ; [2H]c1c([2H])c(-c2c3ccccc3c(-c3ccc4ccccc4c3)c3ccccc23)c2c(oc3c4c([2H])c([2H])c([2H])c([2H])c4c([2H])c([2H])c32)c1[2H] ; [2H]c1c([2H])c(-c2c3ccccc3c(-c3ccc4ccccc4c3)c3ccccc23)c2c(oc3c([2H])c4c([2H])c([2H])c([2H])c([2H])c4c([2H])c32)c1[2H]]

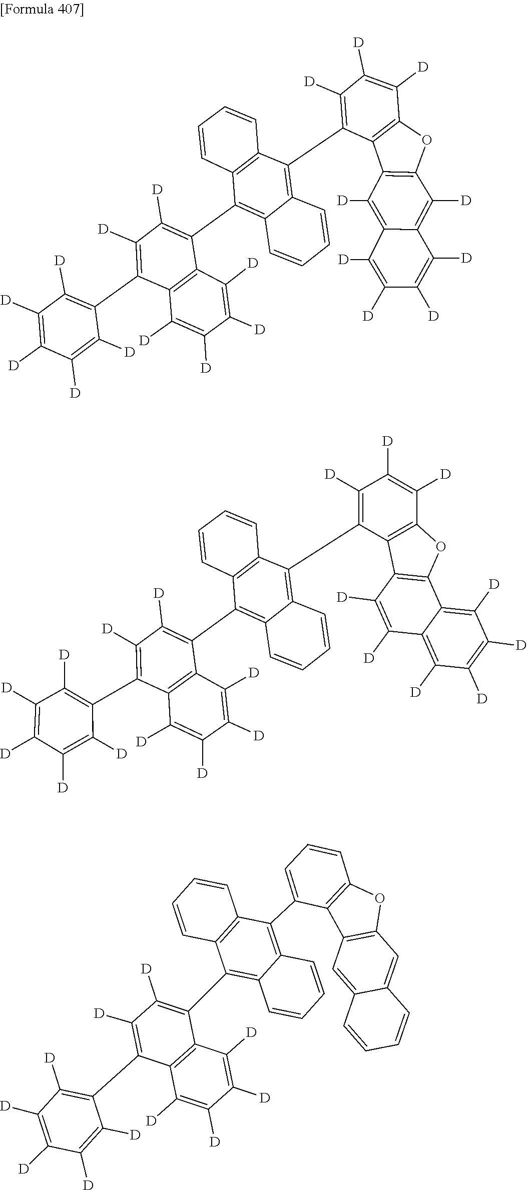

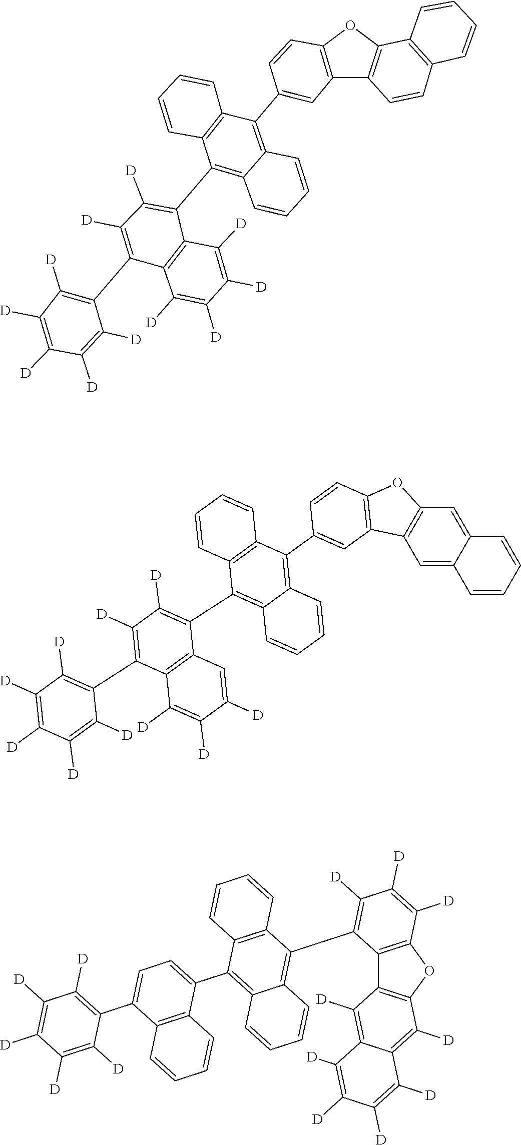 [SMILES: [2H]c1c([2H])c([2H])c(-c2c([2H])c([2H])c(-c3c4ccccc4c(-c4ccc5oc6c7ccccc7ccc6c5c4)c4ccccc34)c3c([2H])c([2H])c([2H])c([2H])c23)c([2H])c1[2H] ; [2H]c1cc2c(-c3c4ccccc4c(-c4ccc5oc6cc7ccccc7cc6c5c4)c4ccccc34)c([2H])c([2H])c(-c3c([2H])c([2H])c([2H])c([2H])c3[2H])c2c([2H])c1[2H] ; [2H]c1c([2H])c([2H])c(-c2ccc(-c3c4ccccc4c(-c4c([2H])c([2H])c([2H])c5oc6c([2H])c7c([2H])c([2H])c([2H])c([2H])c7c([2H])c6c45)c4ccccc34)c3ccccc23)c([2H])c1[2H]]

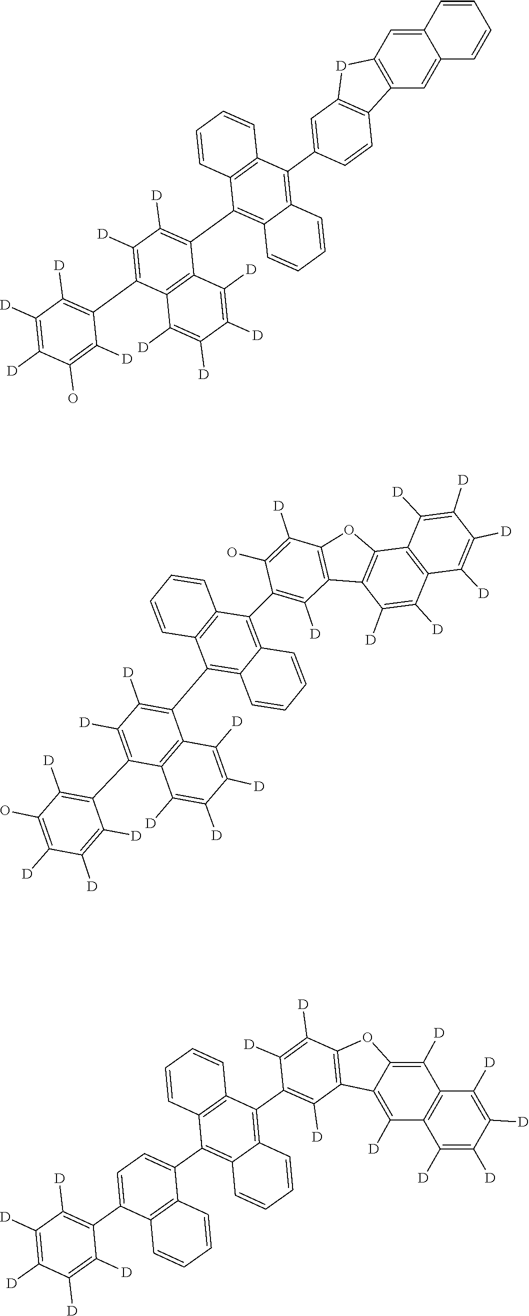

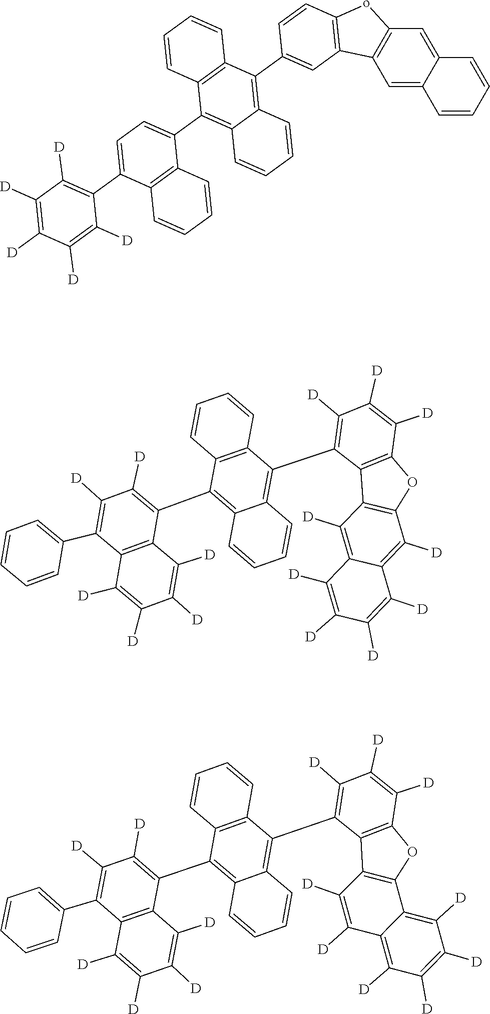

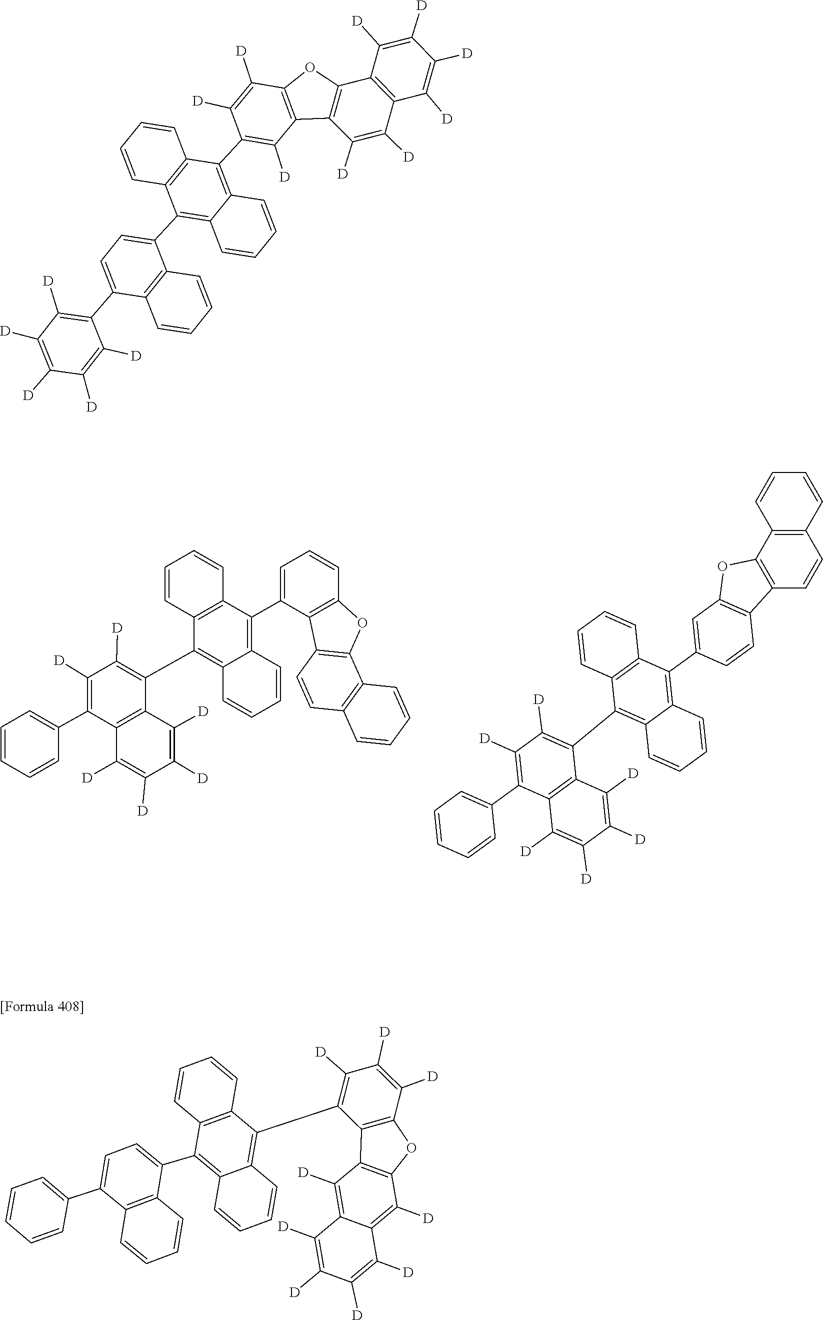

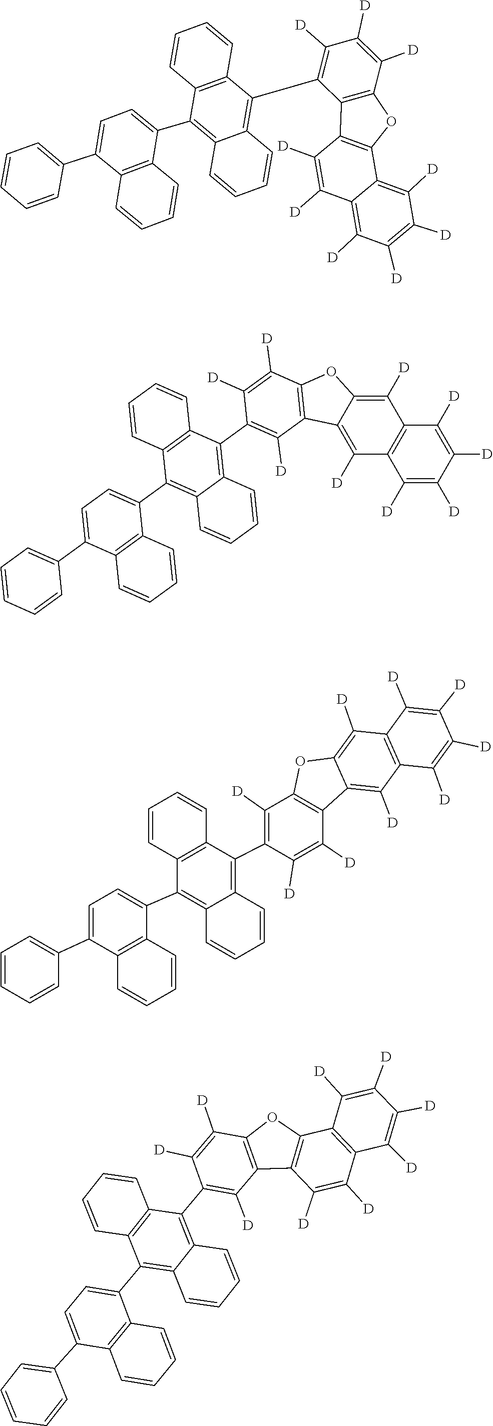

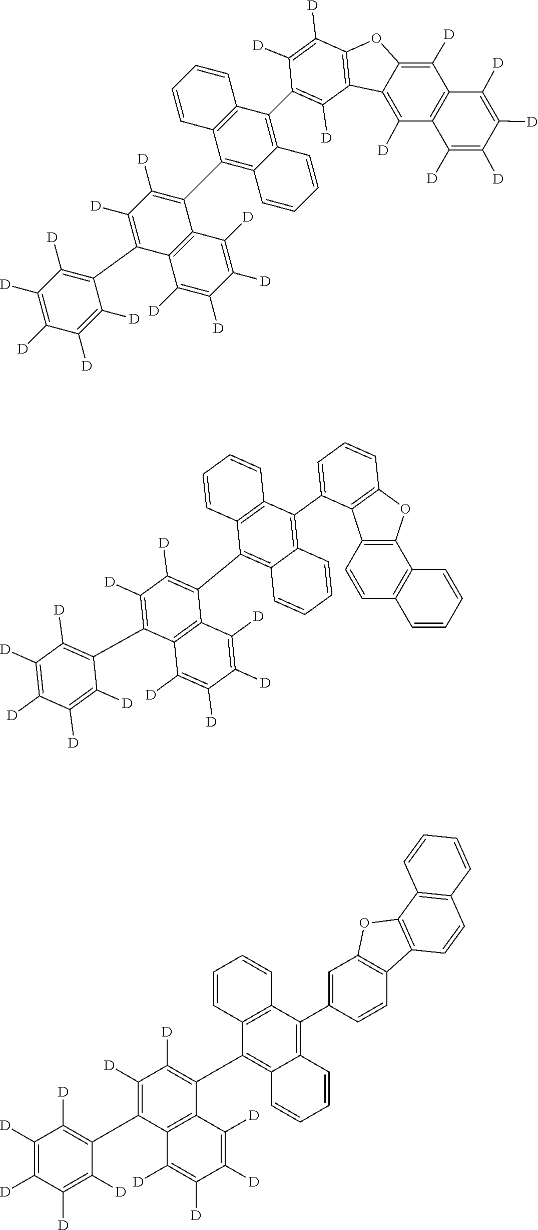 [SMILES: [2H]c1c([2H])c([2H])c(-c2c([2H])c([2H])c(-c3c4ccccc4c(-c4c([2H])c([2H])c5oc6c([2H])c7c([2H])c([2H])c([2H])c([2H])c7c([2H])c6c5c4[2H])c4ccccc34)c3c([2H])c([2H])c([2H])c([2H])c23)c([2H])c1[2H] ; [2H]c1c([2H])c([2H])c(-c2c([2H])c([2H])c(-c3c4ccccc4c(-c4cccc5oc6c7ccccc7ccc6c45)c4ccccc34)c3c([2H])c([2H])c([2H])c([2H])c23)c([2H])c1[2H] ; [2H]c1c([2H])c([2H])c(-c2c([2H])c([2H])c(-c3c4ccccc4c(-c4ccc5c(c4)oc4c6ccccc6ccc54)c4ccccc34)c3c([2H])c([2H])c([2H])c([2H])c23)c([2H])c1[2H]]

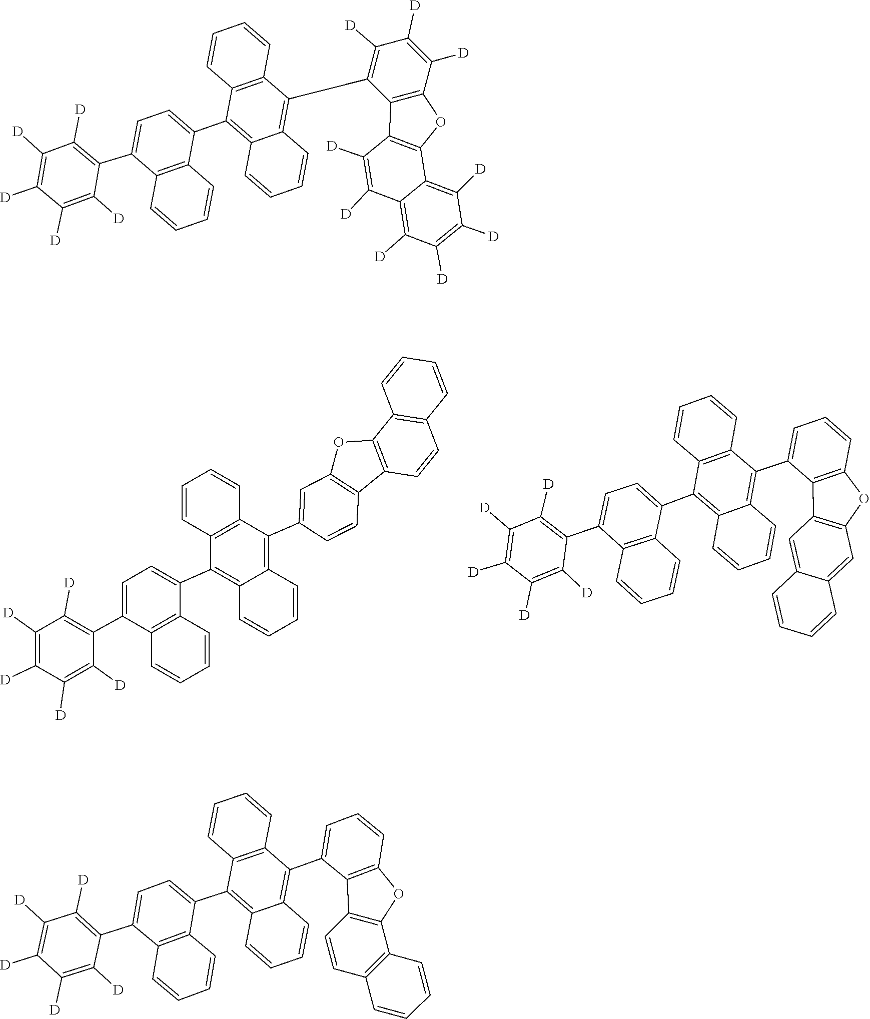 [SMILES: [2H]c1c([2H])c([2H])c(-c2ccc(-c3c4ccccc4c(-c4c([2H])c([2H])c([2H])c5oc6c7c([2H])c([2H])c([2H])c([2H])c7c([2H])c([2H])c6c45)c4ccccc34)c3ccccc23)c([2H])c1[2H] ; [2H]c1c([2H])c([2H])c(-c2ccc(-c3c4ccccc4c(-c4ccc5c(c4)oc4c6ccccc6ccc54)c4ccccc34)c3ccccc23)c([2H])c1[2H] ; [2H]c1c([2H])c([2H])c(-c2ccc(-c3c4ccccc4c(-c4cccc5oc6cc7ccccc7cc6c45)c4ccccc34)c3ccccc23)c([2H])c1[2H] ; [2H]c1c([2H])c([2H])c(-c2ccc(-c3c4ccccc4c(-c4cccc5oc6c7ccccc7ccc6c45)c4ccccc34)c3ccccc23)c([2H])c1[2H]]

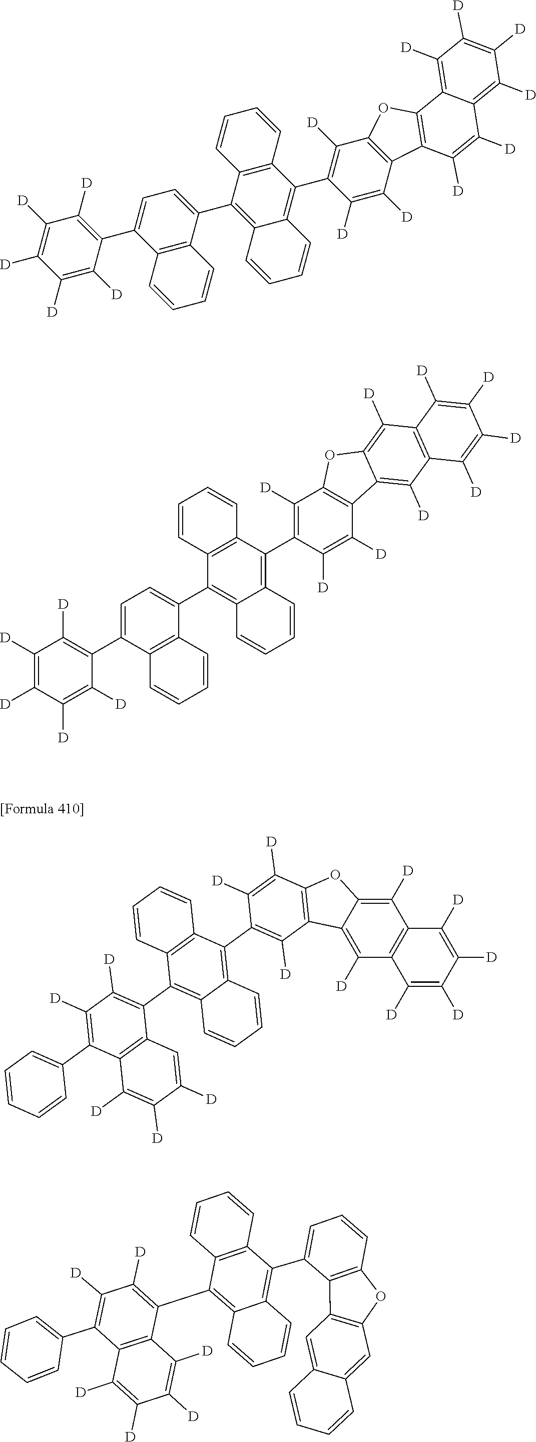

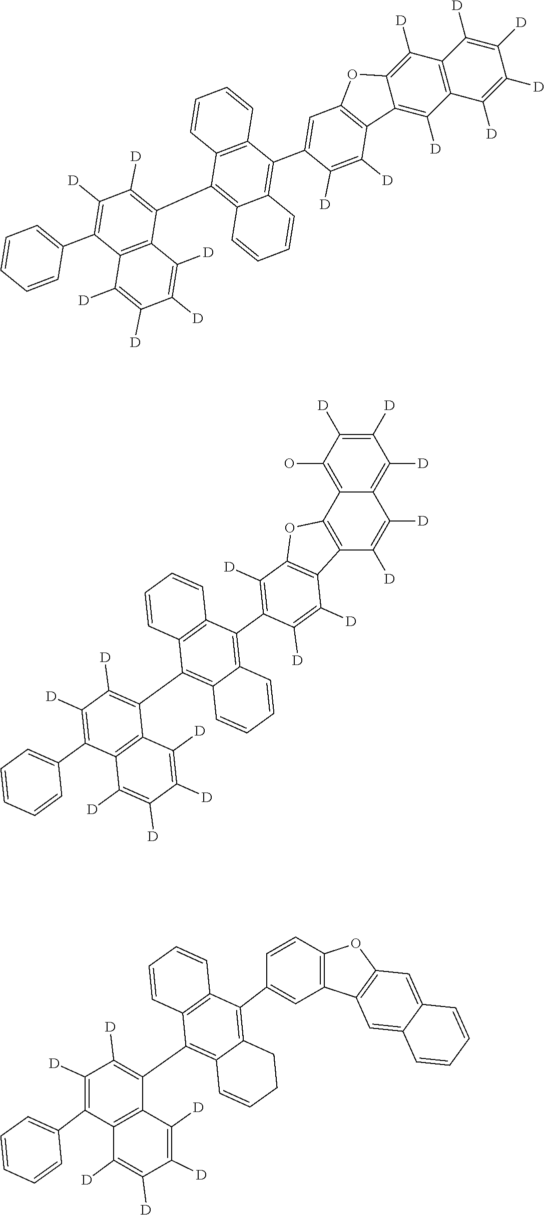

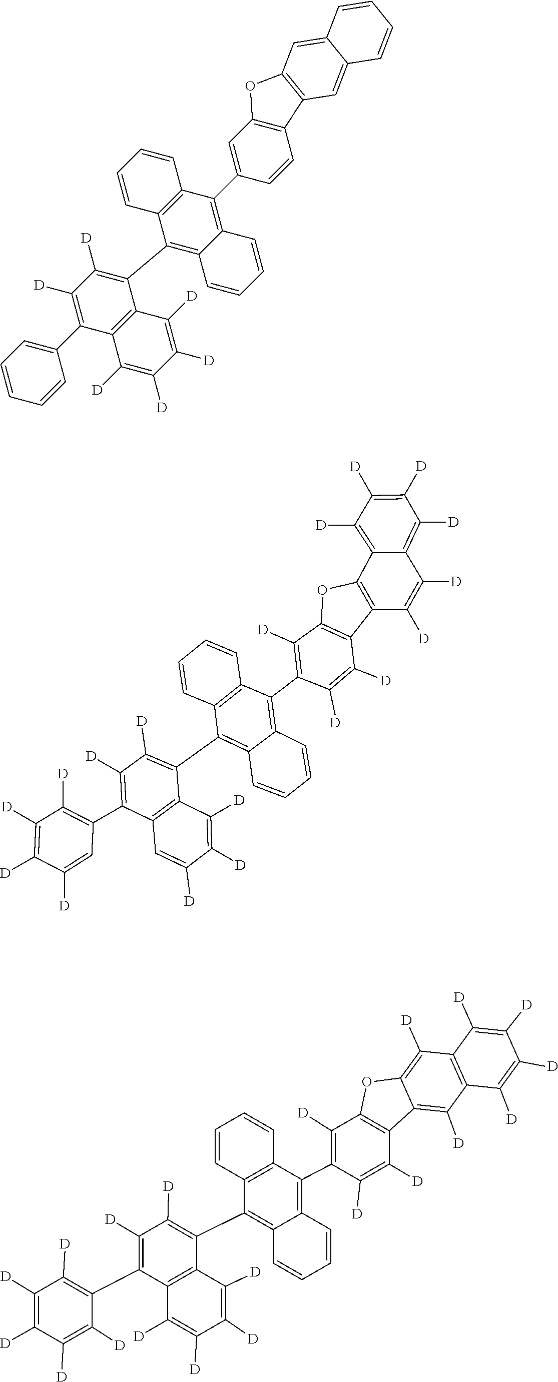 [SMILES: [2H]c1c([2H])c([2H])c2c(-c3c4ccccc4c(-c4ccc5c(c4)oc4cc6ccccc6cc45)c4ccccc34)c([2H])c([2H])c(-c3ccccc3)c2c1[2H] ; [2H]c1cc(-c2c([2H])c([2H])c(-c3c4ccccc4c(-c4c([2H])c([2H])c5c(oc6c7c([2H])c([2H])c([2H])c([2H])c7c([2H])c([2H])c65)c4[2H])c4ccccc34)c3c([2H])c([2H])c([2H])cc23)c([2H])c([2H])c1[2H] ; [2H]c1c([2H])c([2H])c(-c2c([2H])c([2H])c(-c3c4ccccc4c(-c4c([2H])c([2H])c5c(oc6c([2H])c7c([2H])c([2H])c([2H])c([2H])c7c([2H])c65)c4[2H])c4ccccc34)c3c([2H])c([2H])c([2H])c([2H])c23)c([2H])c1[2H]]

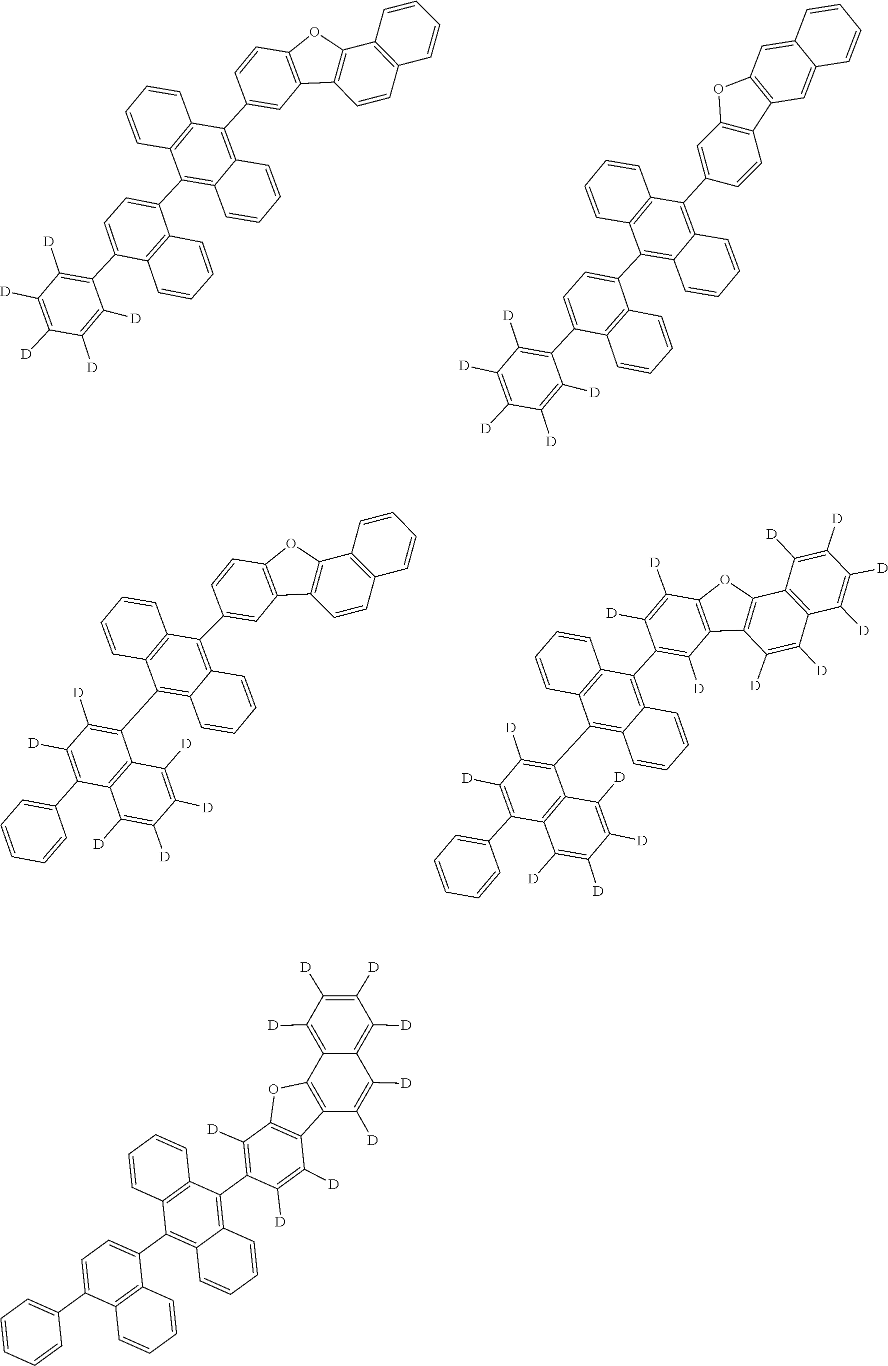

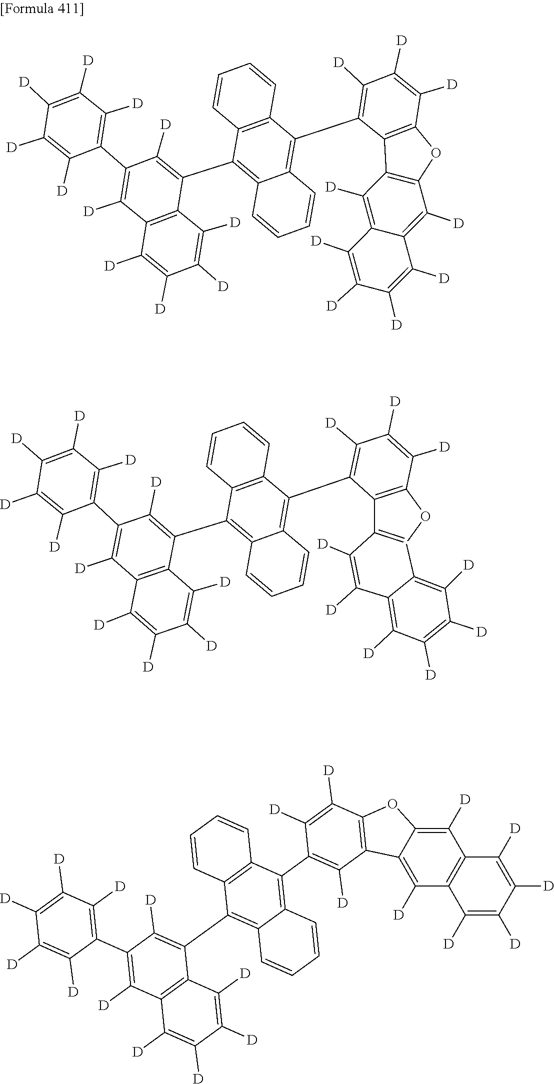

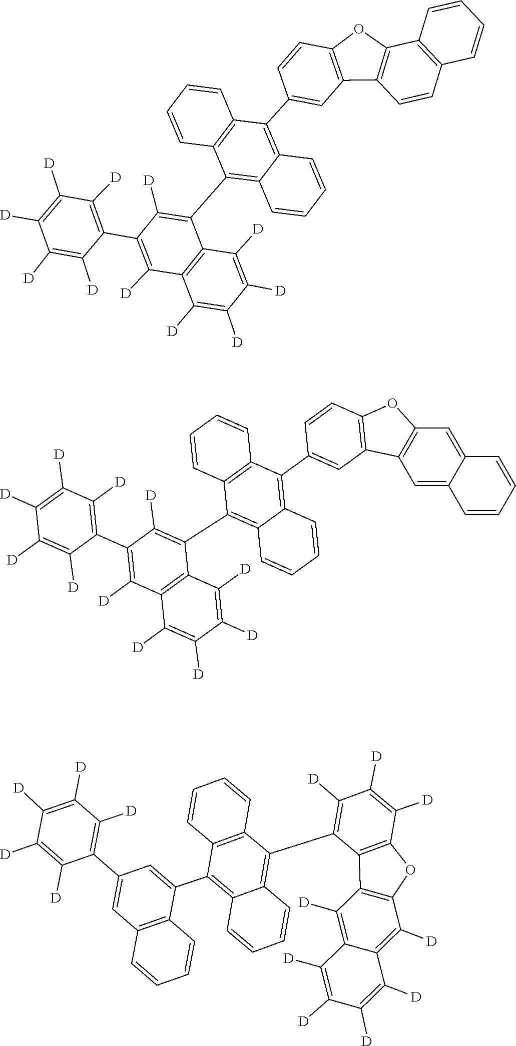 [SMILES: [2H]c1c([2H])c([2H])c(-c2c([2H])c(-c3c4ccccc4c(-c4ccc5oc6c7ccccc7ccc6c5c4)c4ccccc34)c3c([2H])c([2H])c([2H])c([2H])c3c2[2H])c([2H])c1[2H] ; [2H]c1c([2H])c([2H])c(-c2c([2H])c(-c3c4ccccc4c(-c4ccc5oc6cc7ccccc7cc6c5c4)c4ccccc34)c3c([2H])c([2H])c([2H])c([2H])c3c2[2H])c([2H])c1[2H] ; [2H]c1c([2H])c([2H])c(-c2cc(-c3c4ccccc4c(-c4c([2H])c([2H])c([2H])c5oc6c([2H])c7c([2H])c([2H])c([2H])c([2H])c7c([2H])c6c45)c4ccccc34)c3ccccc3c2)c([2H])c1[2H]]

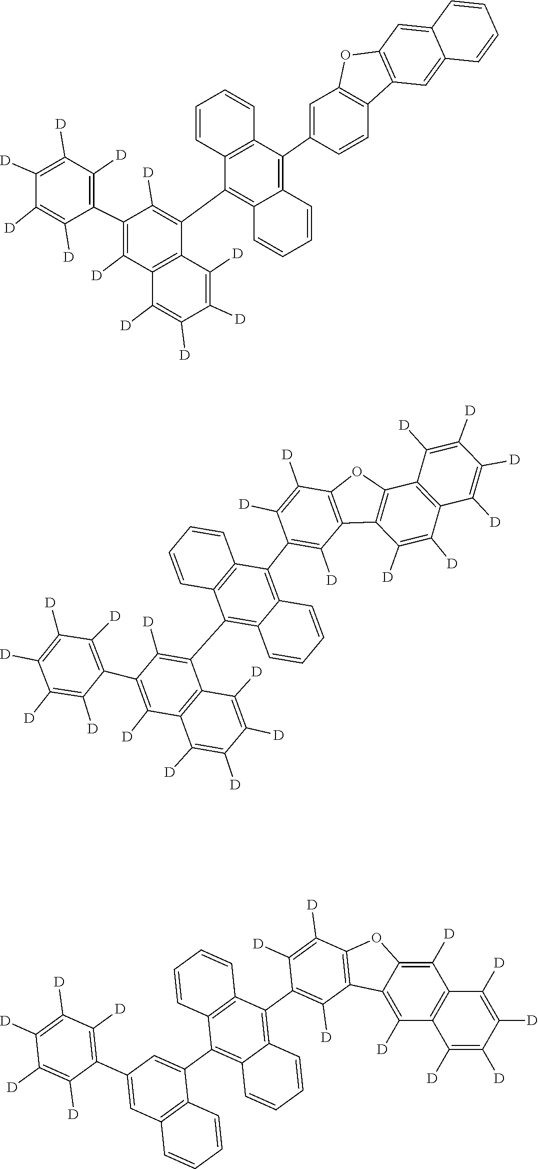 [SMILES: [2H]c1c([2H])c([2H])c(-c2c([2H])c(-c3c4ccccc4c(-c4ccc5c(c4)oc4cc6ccccc6cc45)c4ccccc34)c3c([2H])c([2H])c([2H])c([2H])c3c2[2H])c([2H])c1[2H] ; [2H]c1c([2H])c([2H])c(-c2c([2H])c(-c3c4ccccc4c(-c4c([2H])c([2H])c5oc6c7c([2H])c([2H])c([2H])c([2H])c7c([2H])c([2H])c6c5c4[2H])c4ccccc34)c3c([2H])c([2H])c([2H])c([2H])c3c2[2H])c([2H])c1[2H] ; [2H]c1c([2H])c([2H])c(-c2cc(-c3c4ccccc4c(-c4c([2H])c([2H])c5oc6c([2H])c7c([2H])c([2H])c([2H])c([2H])c7c([2H])c6c5c4[2H])c4ccccc34)c3ccccc3c2)c([2H])c1[2H]]

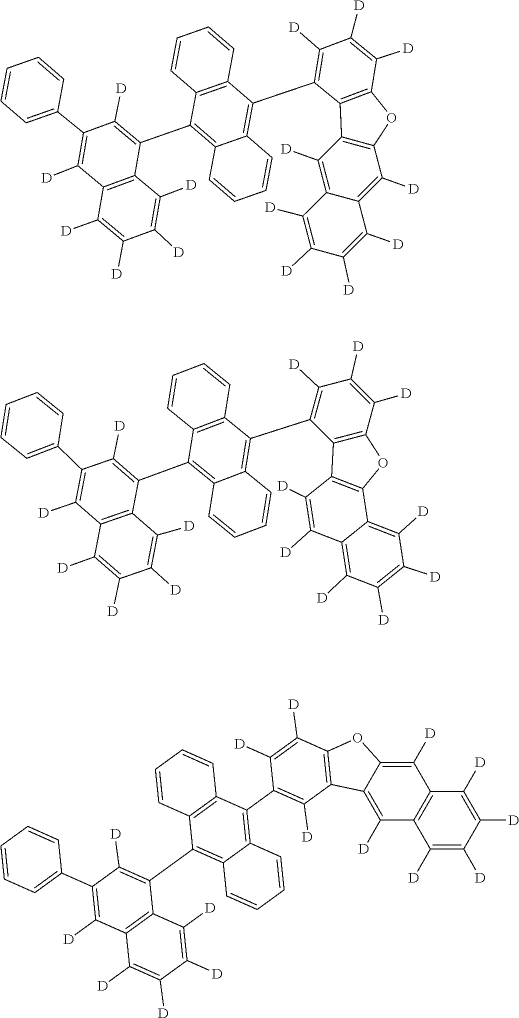 [SMILES: [2H]c1c([2H])c(-c2c3ccccc3c(-c3c([2H])c(-c4ccccc4)c([2H])c4c([2H])c([2H])c([2H])c([2H])c34)c3ccccc23)c2c(oc3c([2H])c4c([2H])c([2H])c([2H])c([2H])c4c([2H])c32)c1[2H] ; [2H]c1c([2H])c(-c2c3ccccc3c(-c3c([2H])c(-c4ccccc4)c([2H])c4c([2H])c([2H])c([2H])c([2H])c34)c3ccccc23)c2c(oc3c4c([2H])c([2H])c([2H])c([2H])c4c([2H])c([2H])c32)c1[2H] ; [2H]c1c(-c2c3ccccc3c(-c3c([2H])c(-c4ccccc4)c([2H])c4c([2H])c([2H])c([2H])c([2H])c34)c3ccccc23)c([2H])c2c(oc3c([2H])c4c([2H])c([2H])c([2H])c([2H])c4c([2H])c32)c1[2H]]

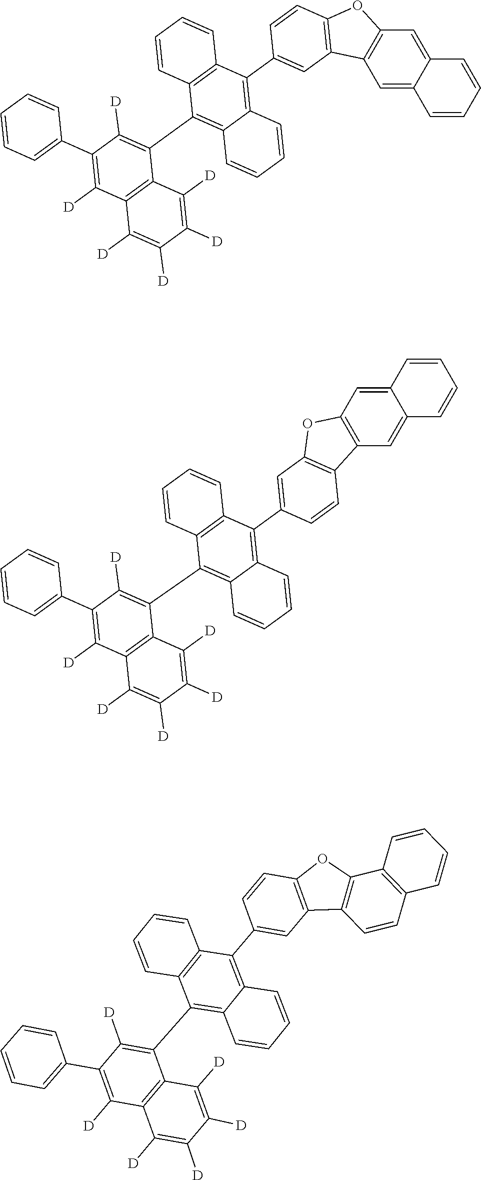 [SMILES: [2H]c1c([2H])c([2H])c2c(-c3c4ccccc4c(-c4ccc5oc6cc7ccccc7cc6c5c4)c4ccccc34)c([2H])c(-c3ccccc3)c([2H])c2c1[2H] ; [2H]c1c([2H])c([2H])c2c(-c3c4ccccc4c(-c4ccc5c(c4)oc4cc6ccccc6cc45)c4ccccc34)c([2H])c(-c3ccccc3)c([2H])c2c1[2H] ; [2H]c1c([2H])c([2H])c2c(-c3c4ccccc4c(-c4ccc5oc6c7ccccc7ccc6c5c4)c4ccccc34)c([2H])c(-c3ccccc3)c([2H])c2c1[2H]]

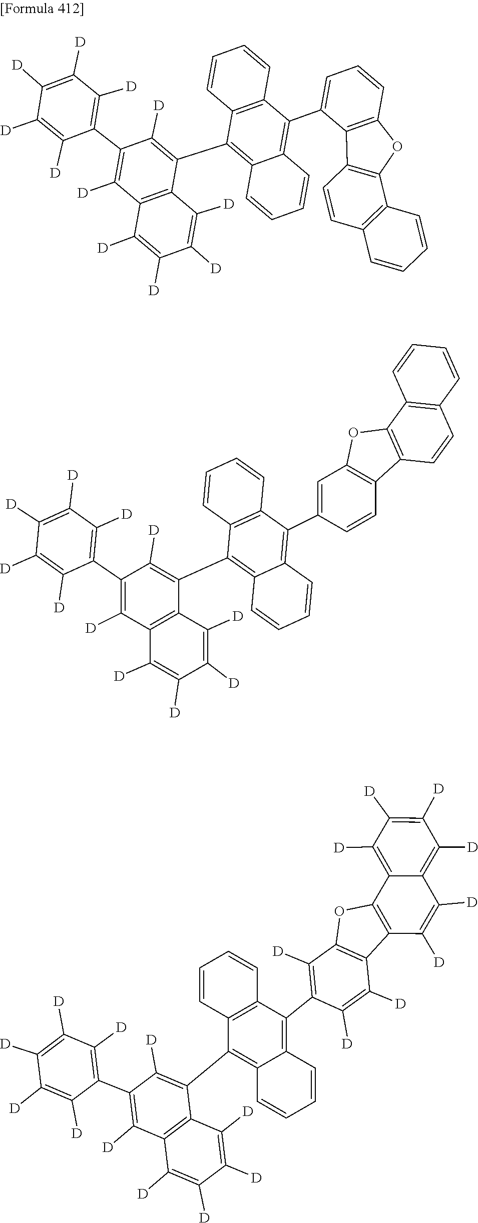 [SMILES: [2H]c1c([2H])c([2H])c(-c2c([2H])c(-c3c4ccccc4c(-c4cccc5oc6c7ccccc7ccc6c45)c4ccccc34)c3c([2H])c([2H])c([2H])c([2H])c3c2[2H])c([2H])c1[2H] ; [2H]c1c([2H])c([2H])c(-c2c([2H])c(-c3c4ccccc4c(-c4ccc5c(c4)oc4c6ccccc6ccc54)c4ccccc34)c3c([2H])c([2H])c([2H])c([2H])c3c2[2H])c([2H])c1[2H] ; [2H]c1c([2H])c([2H])c(-c2c([2H])c(-c3c4ccccc4c(-c4c([2H])c([2H])c5c(oc6c7c([2H])c([2H])c([2H])c([2H])c7c([2H])c([2H])c65)c4[2H])c4ccccc34)c3c([2H])c([2H])c([2H])c([2H])c3c2[2H])c([2H])c1[2H]]

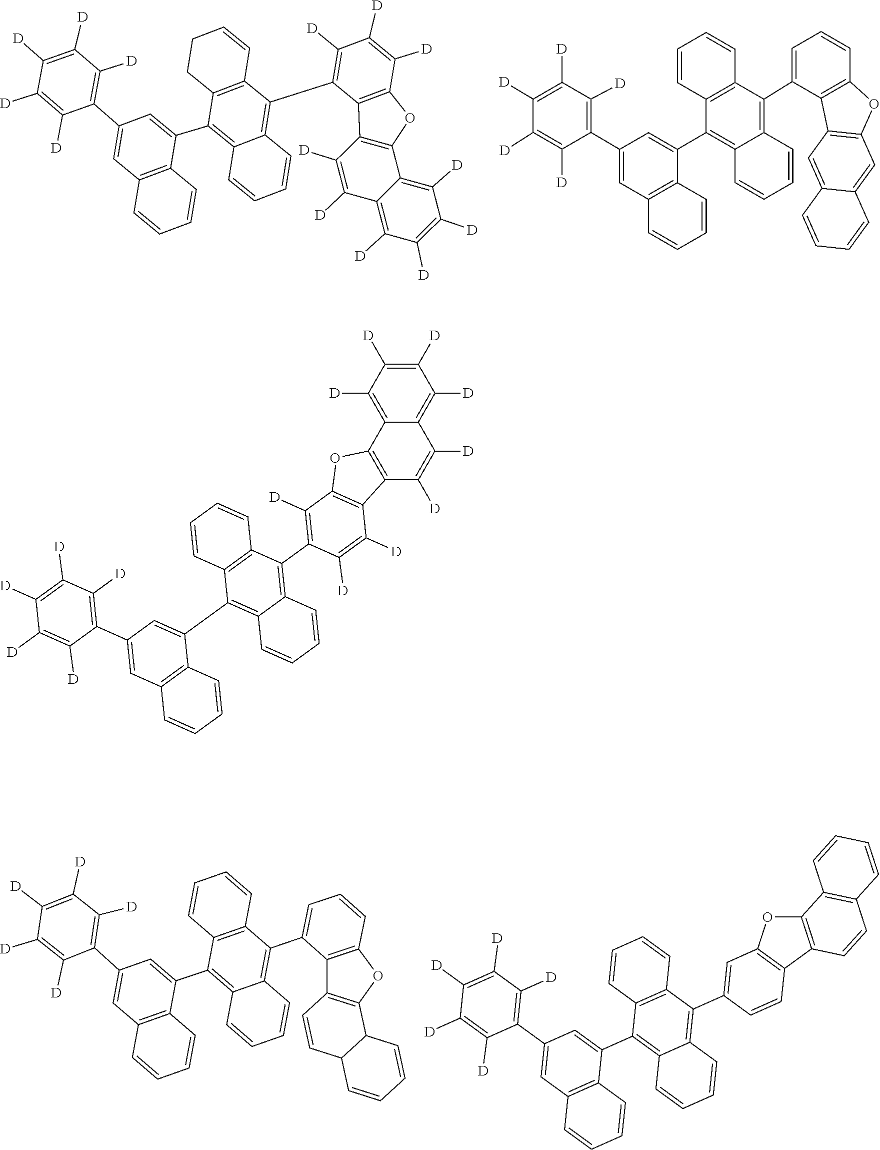

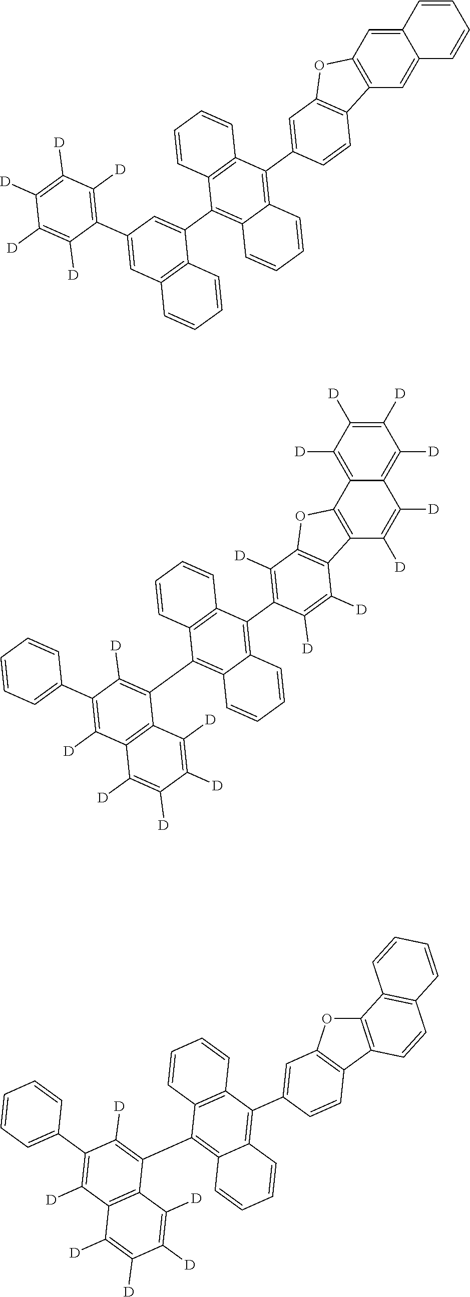 [SMILES: [2H]c1c([2H])c([2H])c(-c2cc(-c3c4ccccc4c(-c4ccc5c(c4)oc4cc6ccccc6cc45)c4ccccc34)c3ccccc3c2)c([2H])c1[2H] ; [2H]c1c([2H])c([2H])c2c(-c3c4ccccc4c(-c4c([2H])c([2H])c5c(oc6c7c([2H])c([2H])c([2H])c([2H])c7c([2H])c([2H])c65)c4[2H])c4ccccc34)c([2H])c(-c3ccccc3)c([2H])c2c1[2H] ; [2H]c1c([2H])c([2H])c2c(-c3c4ccccc4c(-c4ccc5c(c4)oc4c6ccccc6ccc54)c4ccccc34)c([2H])c(-c3ccccc3)c([2H])c2c1[2H]]

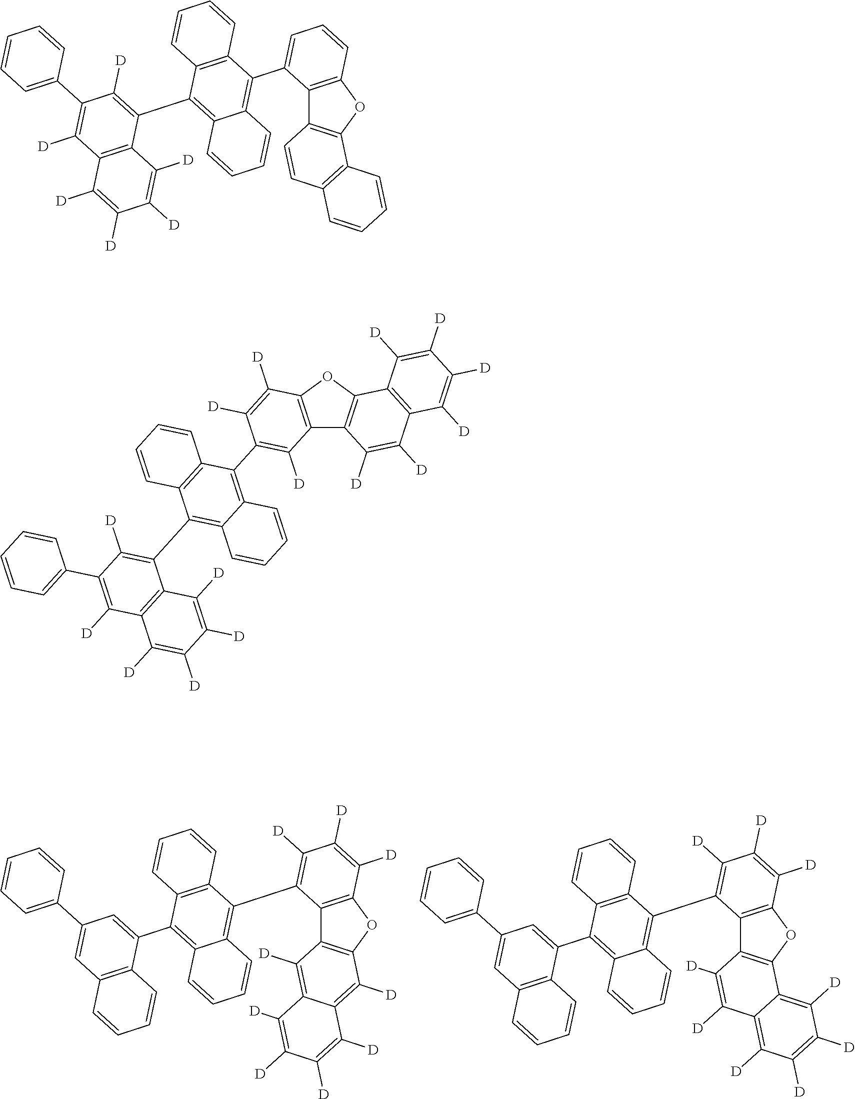

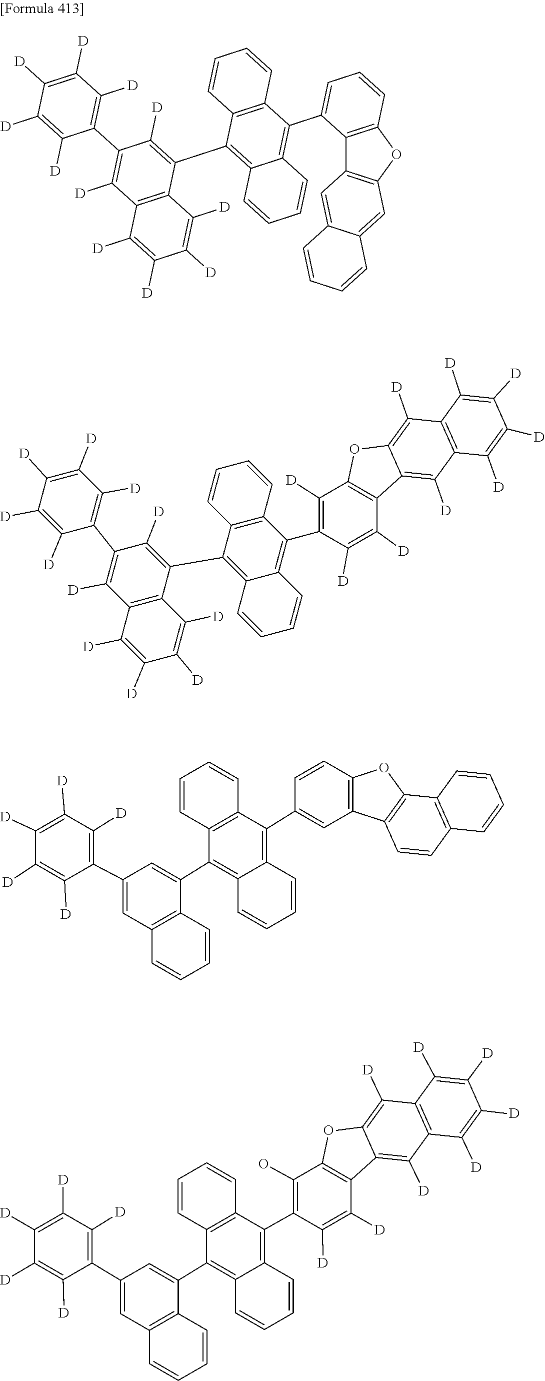

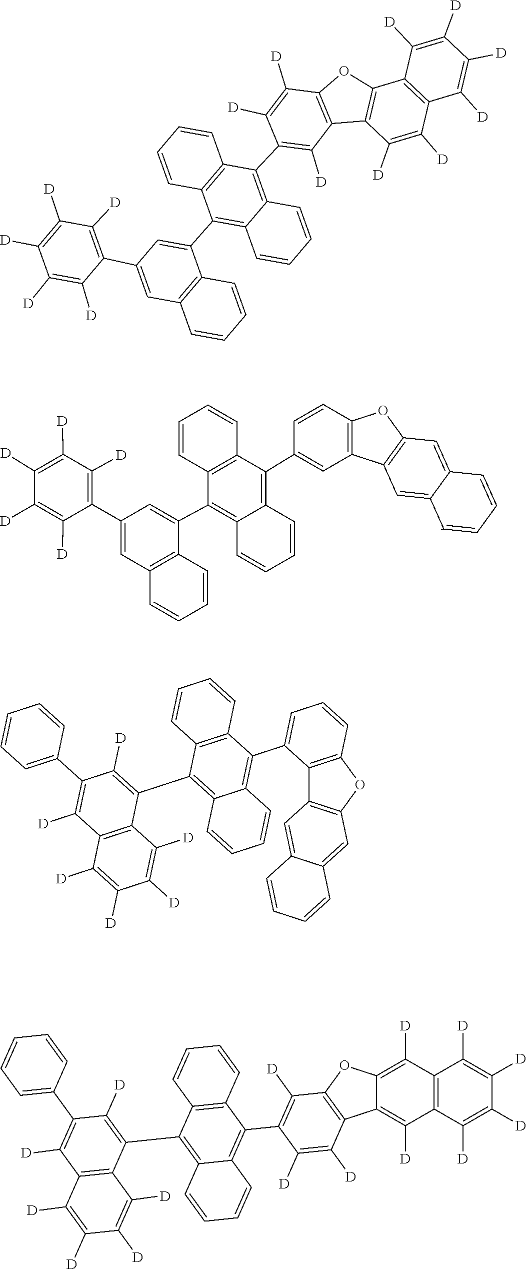

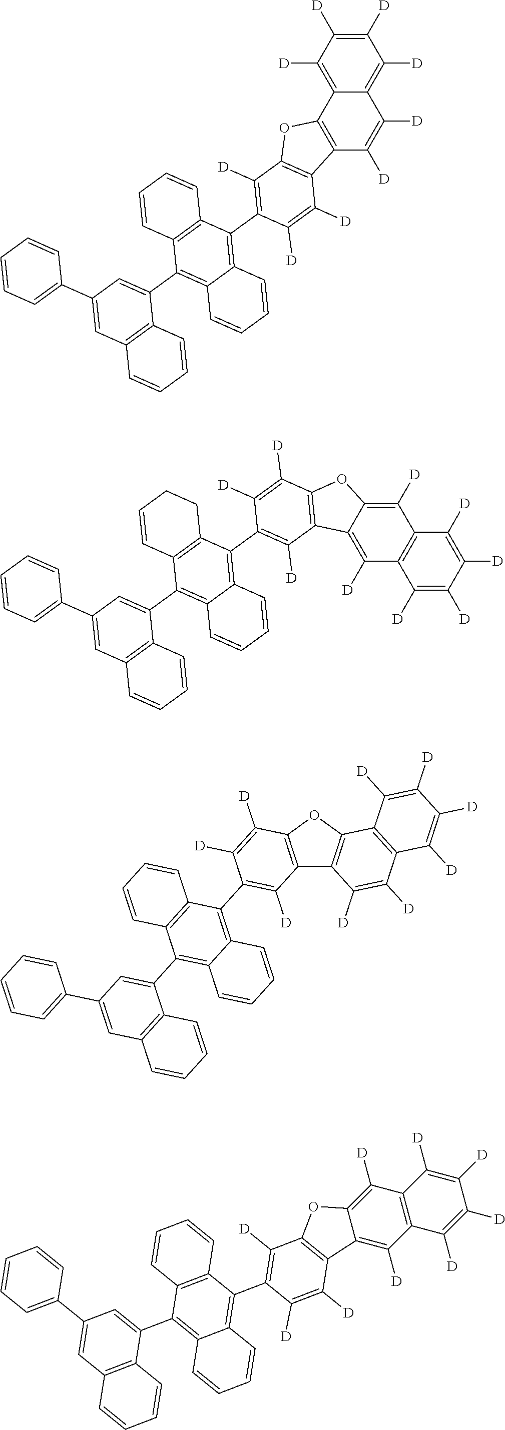

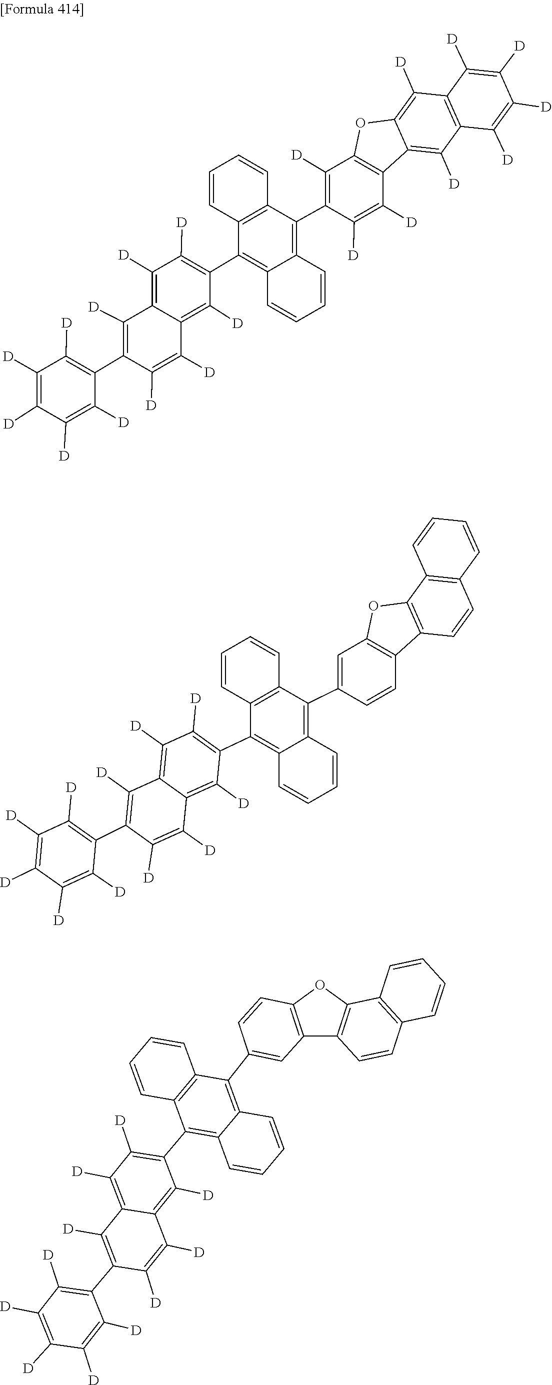 [SMILES: [2H]c1c([2H])c([2H])c(-c2c([2H])c([2H])c3c([2H])c(-c4c5ccccc5c(-c5c([2H])c([2H])c6c(oc7c([2H])c8c([2H])c([2H])c([2H])c([2H])c8c([2H])c76)c5[2H])c5ccccc45)c([2H])c([2H])c3c2[2H])c([2H])c1[2H] ; [2H]c1c([2H])c([2H])c(-c2c([2H])c([2H])c3c([2H])c(-c4c5ccccc5c(-c5ccc6c(c5)oc5c7ccccc7ccc65)c5ccccc45)c([2H])c([2H])c3c2[2H])c([2H])c1[2H] ; [2H]c1c([2H])c([2H])c(-c2c([2H])c([2H])c3c([2H])c(-c4c5ccccc5c(-c5ccc6oc7c8ccccc8ccc7c6c5)c5ccccc45)c([2H])c([2H])c3c2[2H])c([2H])c1[2H]]

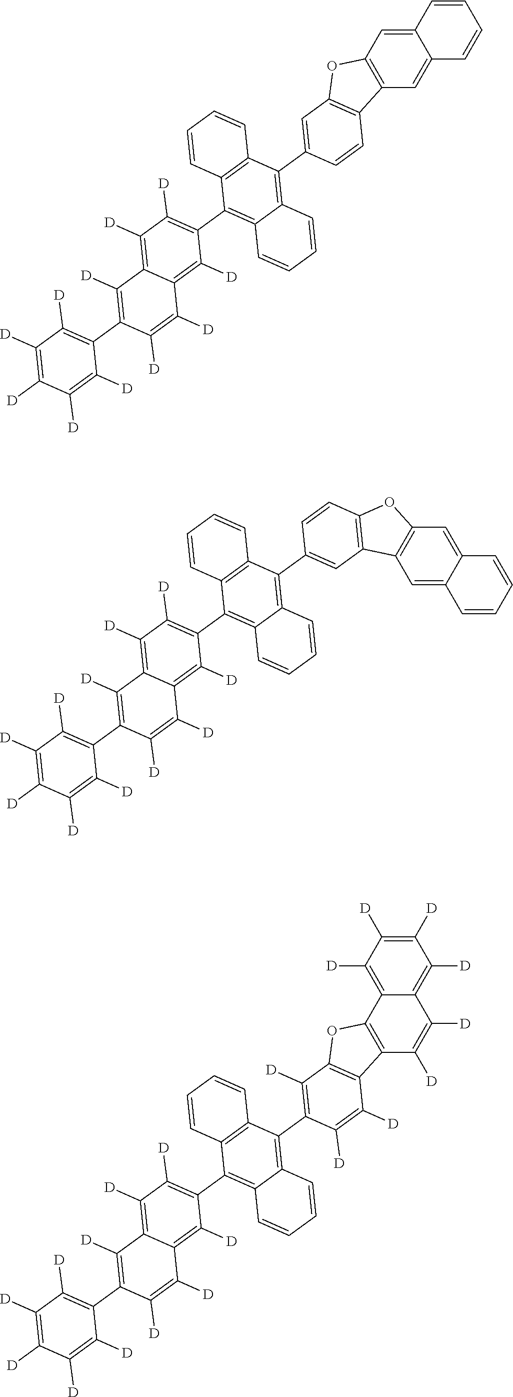 [SMILES: [2H]c1c([2H])c([2H])c(-c2c([2H])c([2H])c3c([2H])c(-c4c5ccccc5c(-c5ccc6c(c5)oc5cc7ccccc7cc56)c5ccccc45)c([2H])c([2H])c3c2[2H])c([2H])c1[2H] ; [2H]c1c([2H])c([2H])c(-c2c([2H])c([2H])c3c([2H])c(-c4c5ccccc5c(-c5ccc6oc7cc8ccccc8cc7c6c5)c5ccccc45)c([2H])c([2H])c3c2[2H])c([2H])c1[2H] ; [2H]c1c([2H])c([2H])c(-c2c([2H])c([2H])c3c([2H])c(-c4c5ccccc5c(-c5c([2H])c([2H])c6c(oc7c8c([2H])c([2H])c([2H])c([2H])c8c([2H])c([2H])c76)c5[2H])c5ccccc45)c([2H])c([2H])c3c2[2H])c([2H])c1[2H]]

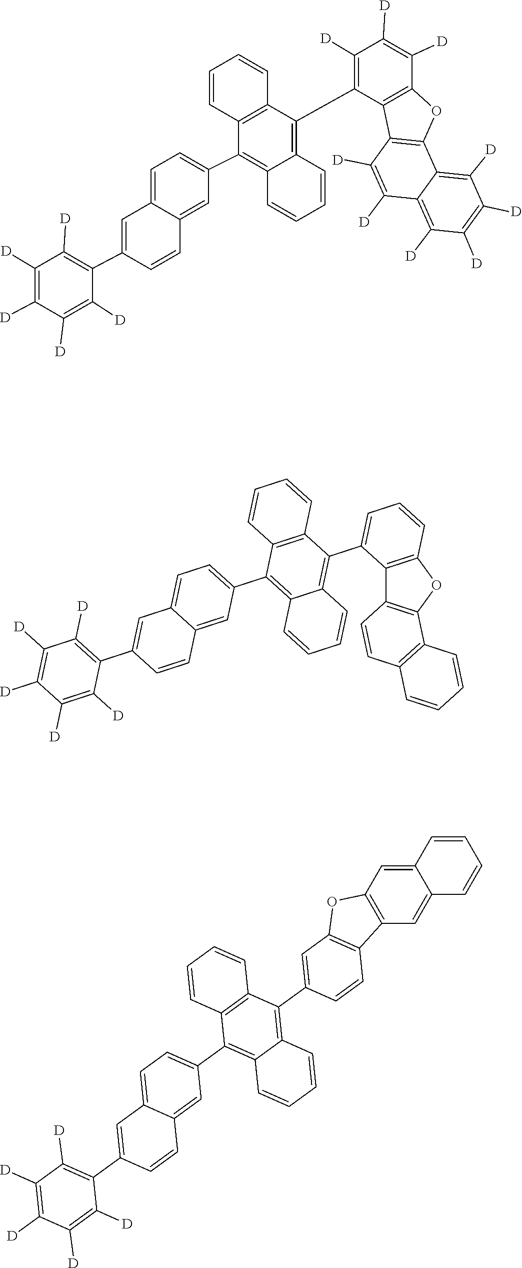 [SMILES: [2H]c1c([2H])c([2H])c(-c2ccc3cc(-c4c5ccccc5c(-c5c([2H])c([2H])c([2H])c6oc7c8c([2H])c([2H])c([2H])c([2H])c8c([2H])c([2H])c7c56)c5ccccc45)ccc3c2)c([2H])c1[2H] ; [2H]c1c([2H])c([2H])c(-c2ccc3cc(-c4c5ccccc5c(-c5cccc6oc7c8ccccc8ccc7c56)c5ccccc45)ccc3c2)c([2H])c1[2H] ; [2H]c1c([2H])c([2H])c(-c2ccc3cc(-c4c5ccccc5c(-c5ccc6c(c5)oc5cc7ccccc7cc56)c5ccccc45)ccc3c2)c([2H])c1[2H]]

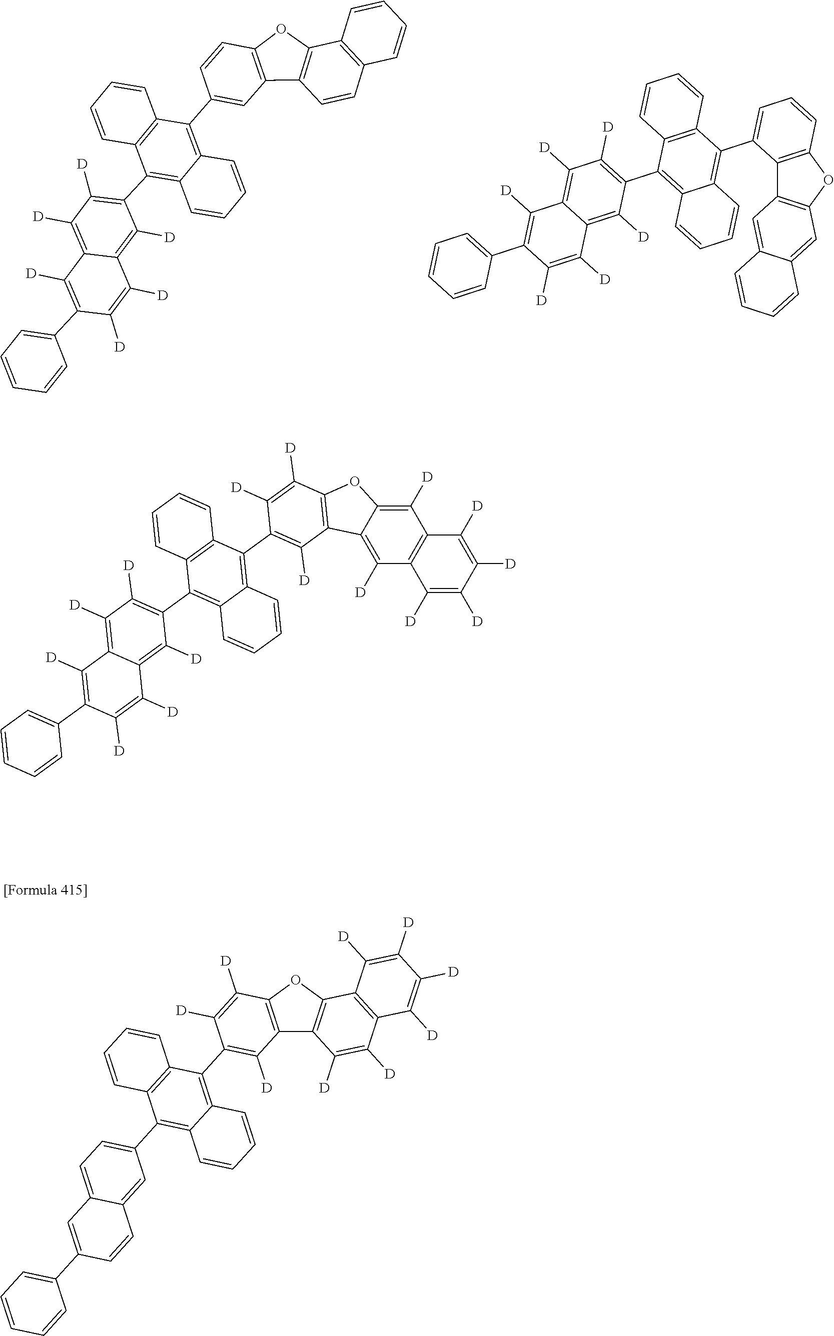

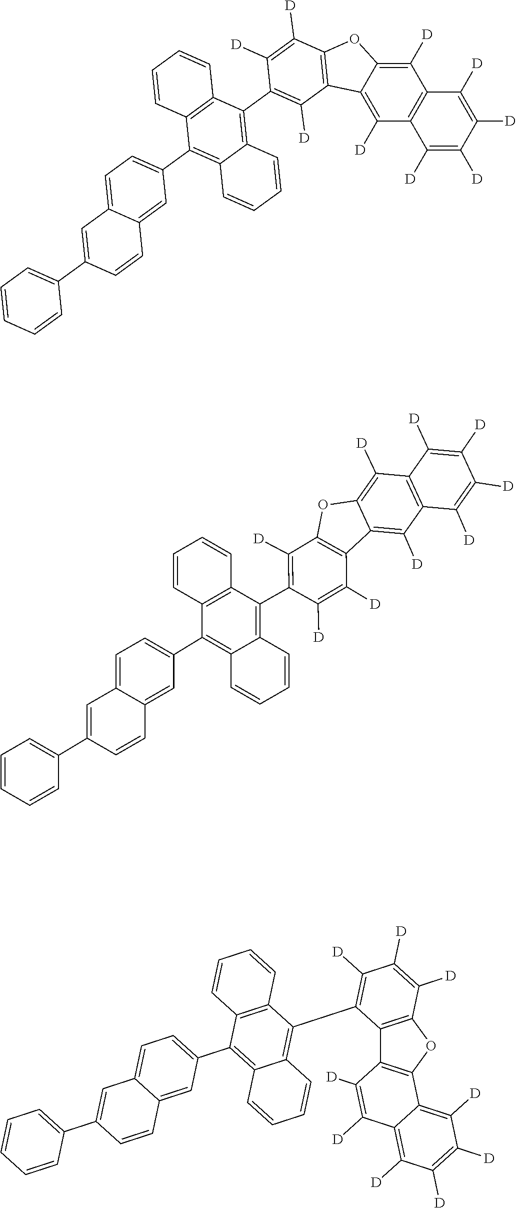 [SMILES: [2H]c1c(-c2c3ccccc3c(-c3ccc4cc(-c5ccccc5)ccc4c3)c3ccccc23)c([2H])c2c(oc3c([2H])c4c([2H])c([2H])c([2H])c([2H])c4c([2H])c32)c1[2H] ; [2H]c1c([2H])c([2H])c2c([2H])c3c(oc4c([2H])c(-c5c6ccccc6c(-c6ccc7cc(-c8ccccc8)ccc7c6)c6ccccc56)c([2H])c([2H])c43)c([2H])c2c1[2H] ; [2H]c1c([2H])c(-c2c3ccccc3c(-c3ccc4cc(-c5ccccc5)ccc4c3)c3ccccc23)c2c(oc3c4c([2H])c([2H])c([2H])c([2H])c4c([2H])c([2H])c32)c1[2H]]

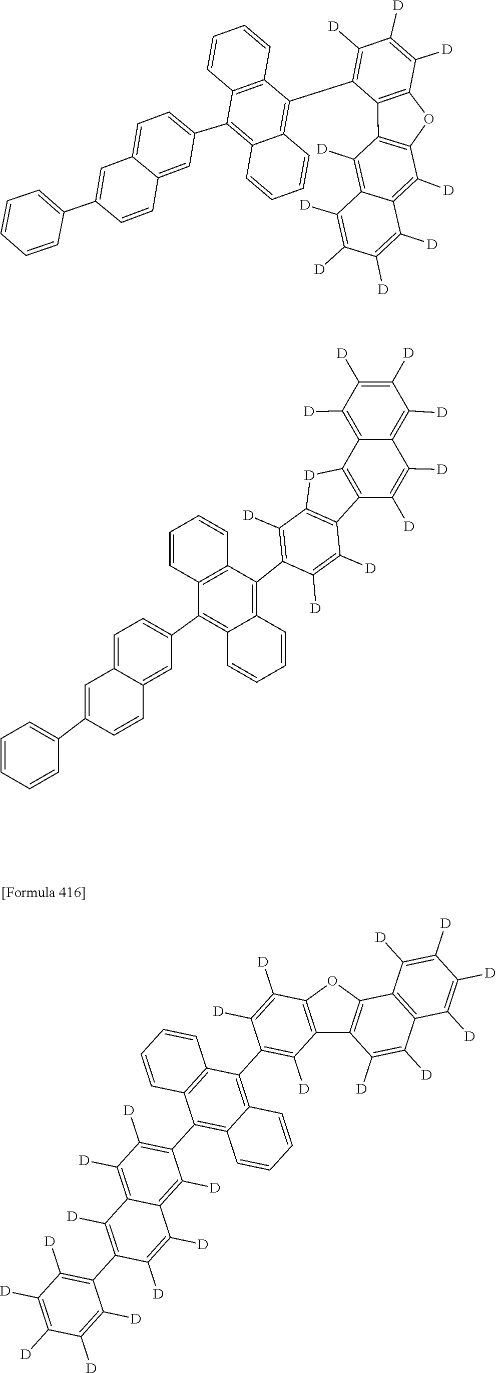

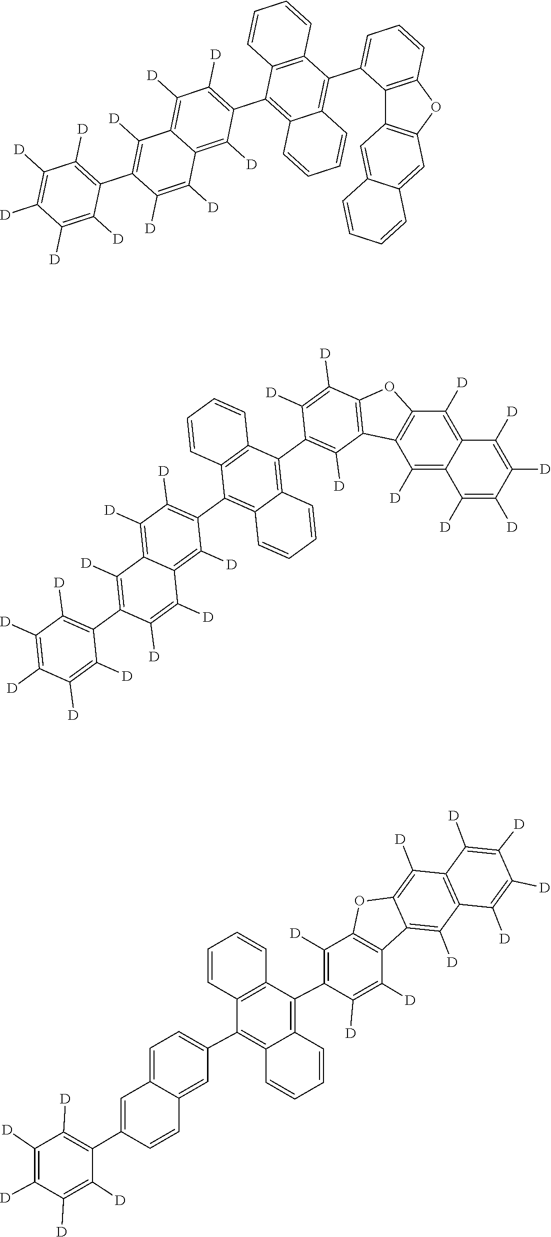 [SMILES: [2H]c1c([2H])c([2H])c(-c2c([2H])c([2H])c3c([2H])c(-c4c5ccccc5c(-c5cccc6oc7cc8ccccc8cc7c56)c5ccccc45)c([2H])c([2H])c3c2[2H])c([2H])c1[2H] ; [2H]c1c([2H])c([2H])c(-c2c([2H])c([2H])c3c([2H])c(-c4c5ccccc5c(-c5c([2H])c([2H])c6oc7c([2H])c8c([2H])c([2H])c([2H])c([2H])c8c([2H])c7c6c5[2H])c5ccccc45)c([2H])c([2H])c3c2[2H])c([2H])c1[2H] ; [2H]c1c([2H])c([2H])c(-c2ccc3cc(-c4c5ccccc5c(-c5c([2H])c([2H])c6c(oc7c([2H])c8c([2H])c([2H])c([2H])c([2H])c8c([2H])c76)c5[2H])c5ccccc45)ccc3c2)c([2H])c1[2H]]

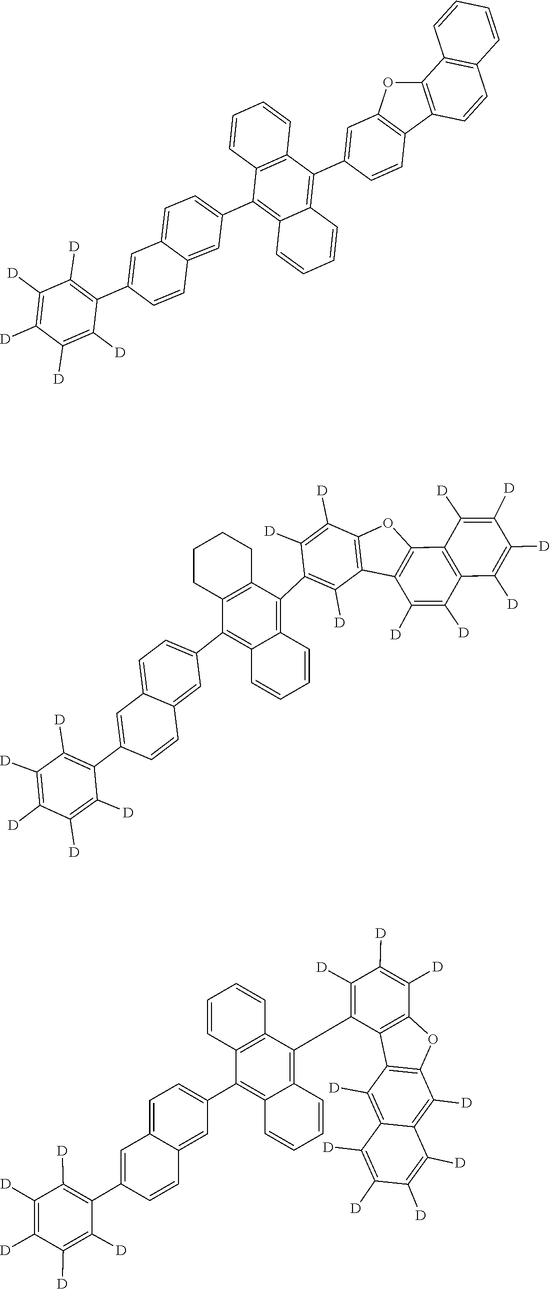 [SMILES: [2H]c1c([2H])c([2H])c(-c2ccc3cc(-c4c5ccccc5c(-c5ccc6c(c5)oc5c7ccccc7ccc65)c5ccccc45)ccc3c2)c([2H])c1[2H] ; [2H]c1c([2H])c([2H])c(-c2ccc3cc(-c4c5c(c(-c6c([2H])c([2H])c7oc8c9c([2H])c([2H])c([2H])c([2H])c9c([2H])c([2H])c8c7c6[2H])c6ccccc46)CCCC5)ccc3c2)c([2H])c1[2H] ; [2H]c1c([2H])c([2H])c(-c2ccc3cc(-c4c5ccccc5c(-c5c([2H])c([2H])c([2H])c6oc7c([2H])c8c([2H])c([2H])c([2H])c([2H])c8c([2H])c7c56)c5ccccc45)ccc3c2)c([2H])c1[2H]]

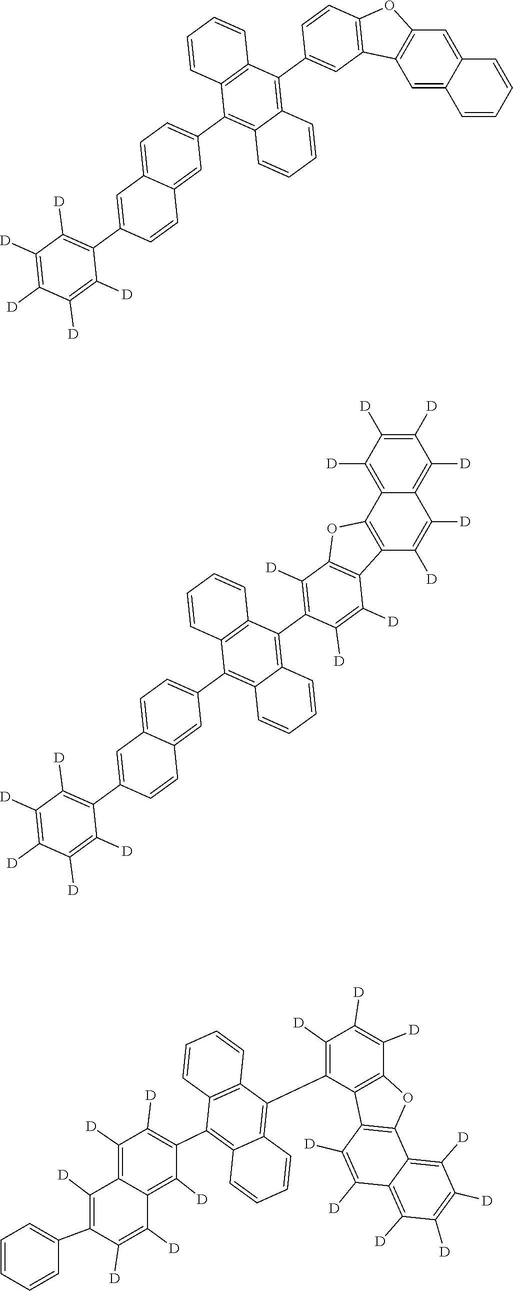 [SMILES: [2H]c1c([2H])c([2H])c(-c2ccc3cc(-c4c5ccccc5c(-c5ccc6oc7cc8ccccc8cc7c6c5)c5ccccc45)ccc3c2)c([2H])c1[2H] ; [2H]c1c([2H])c([2H])c(-c2ccc3cc(-c4c5ccccc5c(-c5c([2H])c([2H])c6c(oc7c8c([2H])c([2H])c([2H])c([2H])c8c([2H])c([2H])c76)c5[2H])c5ccccc45)ccc3c2)c([2H])c1[2H] ; [2H]c1c([2H])c(-c2c3ccccc3c(-c3c([2H])c([2H])c4c([2H])c(-c5ccccc5)c([2H])c([2H])c4c3[2H])c3ccccc23)c2c(oc3c4c([2H])c([2H])c([2H])c([2H])c4c([2H])c([2H])c32)c1[2H]]

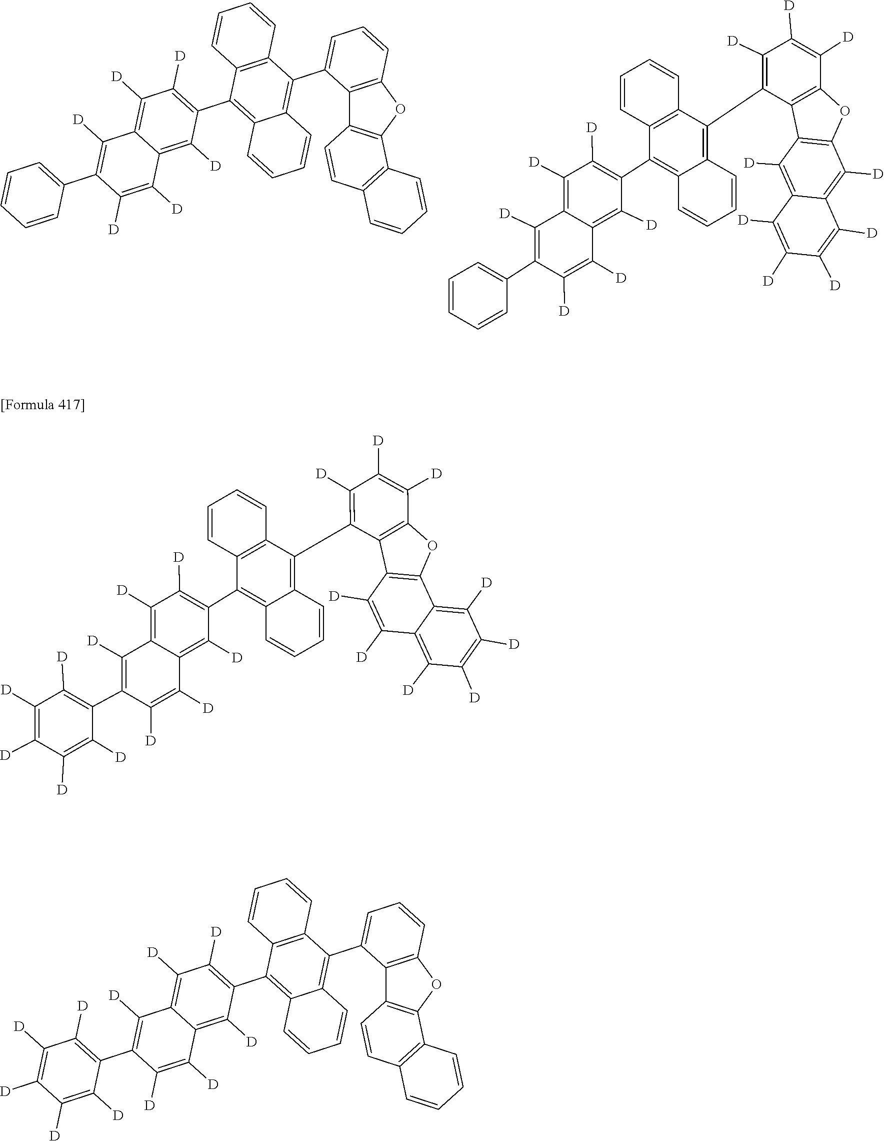

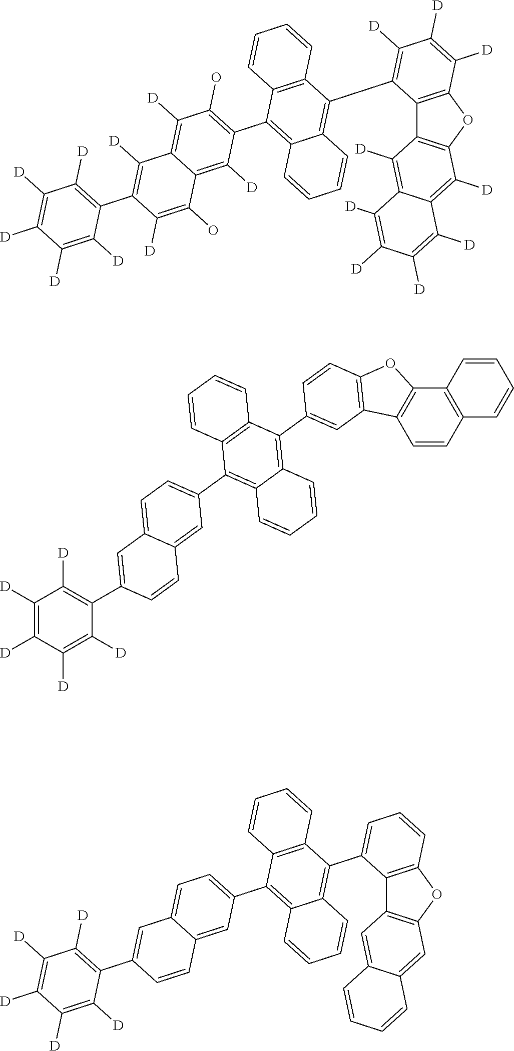 [SMILES: [2H]c1c([2H])c([2H])c(-c2c([2H])c(O)c3c([2H])c(-c4c5ccccc5c(-c5c([2H])c([2H])c([2H])c6oc7c([2H])c8c([2H])c([2H])c([2H])c([2H])c8c([2H])c7c56)c5ccccc45)c(O)c([2H])c3c2[2H])c([2H])c1[2H] ; [2H]c1c([2H])c([2H])c(-c2ccc3cc(-c4c5ccccc5c(-c5ccc6oc7c8ccccc8ccc7c6c5)c5ccccc45)ccc3c2)c([2H])c1[2H] ; [2H]c1c([2H])c([2H])c(-c2ccc3cc(-c4c5ccccc5c(-c5cccc6oc7cc8ccccc8cc7c56)c5ccccc45)ccc3c2)c([2H])c1[2H]]

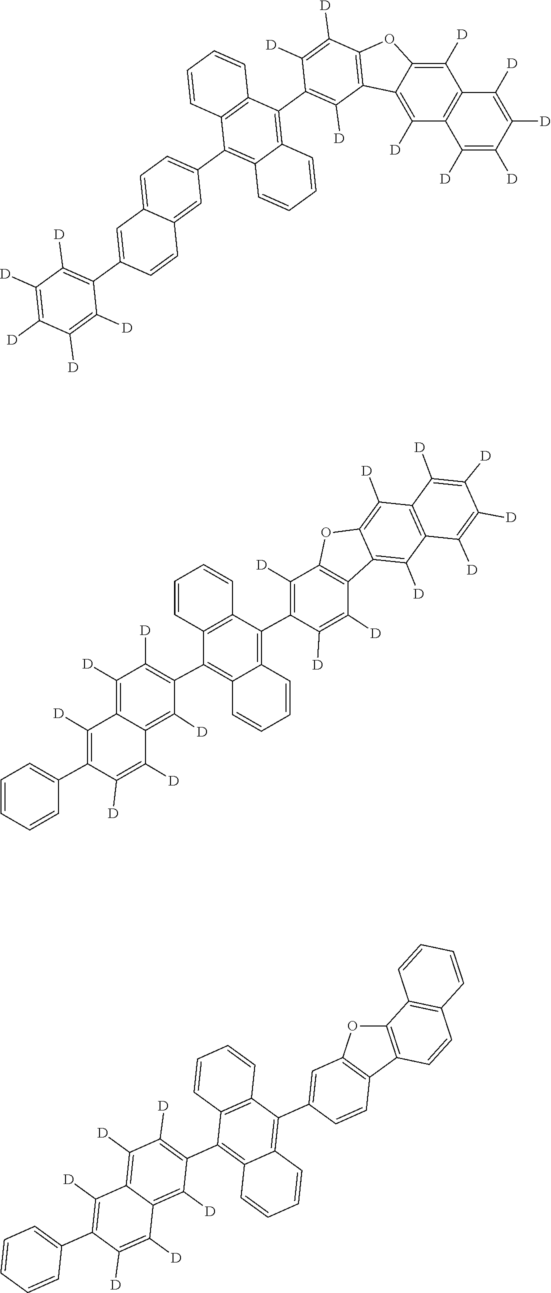 [SMILES: [2H]c1c([2H])c([2H])c(-c2ccc3cc(-c4c5ccccc5c(-c5c([2H])c([2H])c6oc7c([2H])c8c([2H])c([2H])c([2H])c([2H])c8c([2H])c7c6c5[2H])c5ccccc45)ccc3c2)c([2H])c1[2H] ; [2H]c1c(-c2c3ccccc3c(-c3c([2H])c([2H])c4c(oc5c([2H])c6c([2H])c([2H])c([2H])c([2H])c6c([2H])c54)c3[2H])c3ccccc23)c([2H])c2c([2H])c([2H])c(-c3ccccc3)c([2H])c2c1[2H] ; [2H]c1c(-c2c3ccccc3c(-c3ccc4c(c3)oc3c5ccccc5ccc43)c3ccccc23)c([2H])c2c([2H])c([2H])c(-c3ccccc3)c([2H])c2c1[2H]]

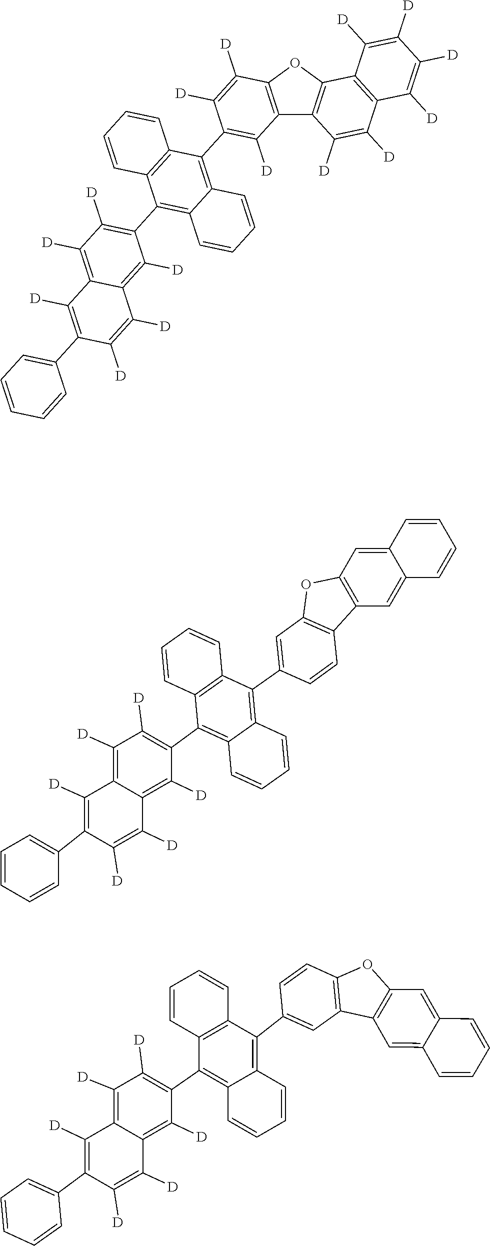 [SMILES: [2H]c1c(-c2c3ccccc3c(-c3c([2H])c([2H])c4c([2H])c(-c5ccccc5)c([2H])c([2H])c4c3[2H])c3ccccc23)c([2H])c2c(oc3c4c([2H])c([2H])c([2H])c([2H])c4c([2H])c([2H])c32)c1[2H] ; [2H]c1c(-c2c3ccccc3c(-c3ccc4c(c3)oc3cc5ccccc5cc34)c3ccccc23)c([2H])c2c([2H])c([2H])c(-c3ccccc3)c([2H])c2c1[2H] ; [2H]c1c(-c2c3ccccc3c(-c3ccc4oc5cc6ccccc6cc5c4c3)c3ccccc23)c([2H])c2c([2H])c([2H])c(-c3ccccc3)c([2H])c2c1[2H]]

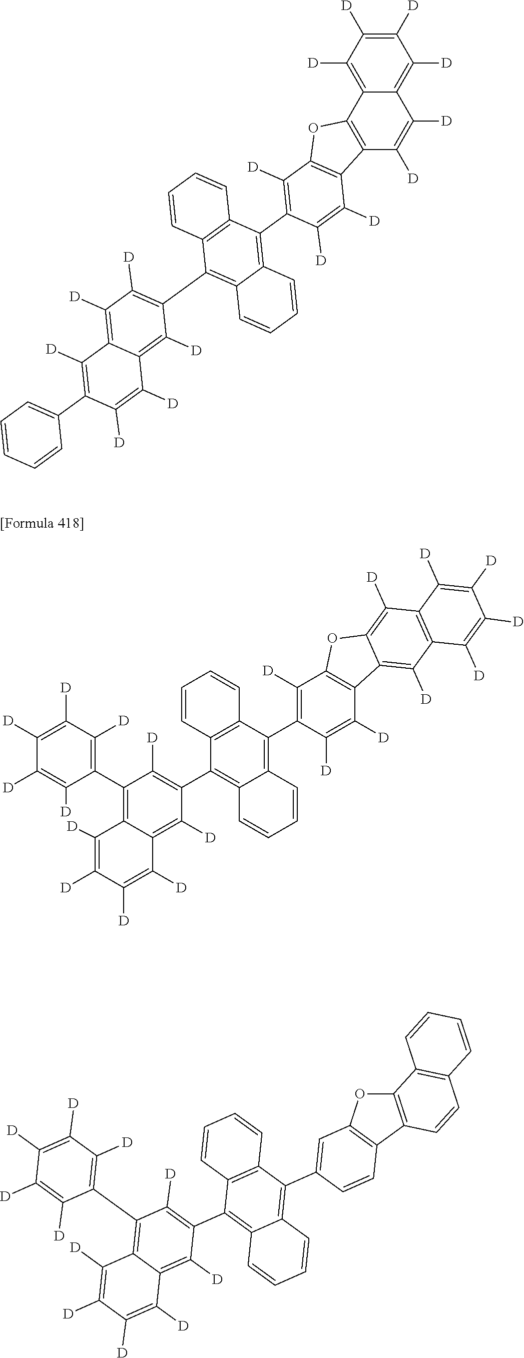 [SMILES: [2H]c1c(-c2c3ccccc3c(-c3c([2H])c([2H])c4c(oc5c6c([2H])c([2H])c([2H])c([2H])c6c([2H])c([2H])c54)c3[2H])c3ccccc23)c([2H])c2c([2H])c([2H])c(-c3ccccc3)c([2H])c2c1[2H] ; [2H]c1c([2H])c([2H])c(-c2c([2H])c(-c3c4ccccc4c(-c4c([2H])c([2H])c5c(oc6c([2H])c7c([2H])c([2H])c([2H])c([2H])c7c([2H])c65)c4[2H])c4ccccc34)c([2H])c3c([2H])c([2H])c([2H])c([2H])c23)c([2H])c1[2H] ; [2H]c1c([2H])c([2H])c(-c2c([2H])c(-c3c4ccccc4c(-c4ccc5c(c4)oc4c6ccccc6ccc54)c4ccccc34)c([2H])c3c([2H])c([2H])c([2H])c([2H])c23)c([2H])c1[2H]]

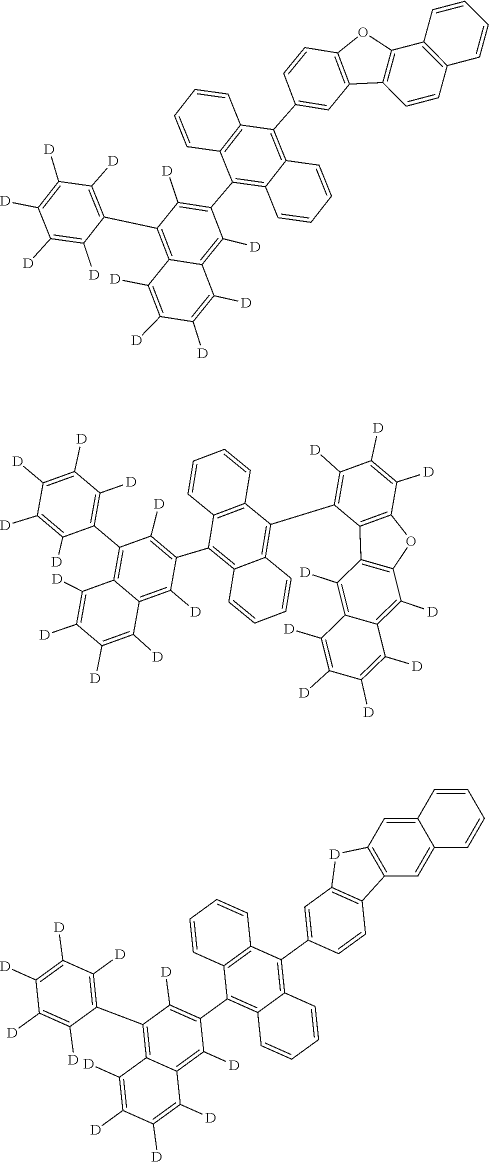

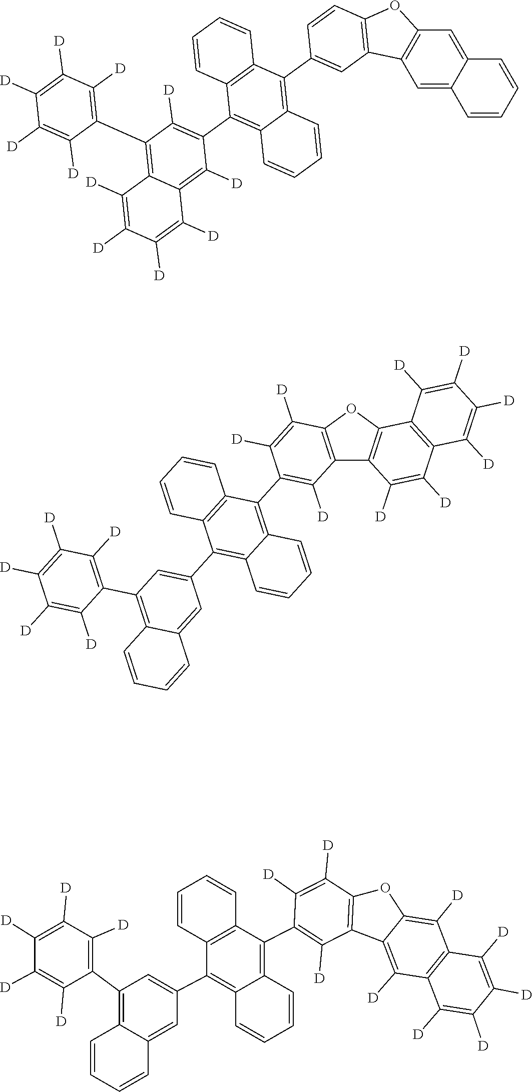 [SMILES: [2H]c1c([2H])c([2H])c(-c2c([2H])c(-c3c4ccccc4c(-c4ccc5oc6cc7ccccc7cc6c5c4)c4ccccc34)c([2H])c3c([2H])c([2H])c([2H])c([2H])c23)c([2H])c1[2H] ; [2H]c1c([2H])c([2H])c(-c2cc(-c3c4ccccc4c(-c4c([2H])c([2H])c5oc6c7c([2H])c([2H])c([2H])c([2H])c7c([2H])c([2H])c6c5c4[2H])c4ccccc34)cc3ccccc23)c([2H])c1[2H] ; [2H]c1c([2H])c([2H])c(-c2cc(-c3c4ccccc4c(-c4c([2H])c([2H])c5oc6c([2H])c7c([2H])c([2H])c([2H])c([2H])c7c([2H])c6c5c4[2H])c4ccccc34)cc3ccccc23)c([2H])c1[2H]]

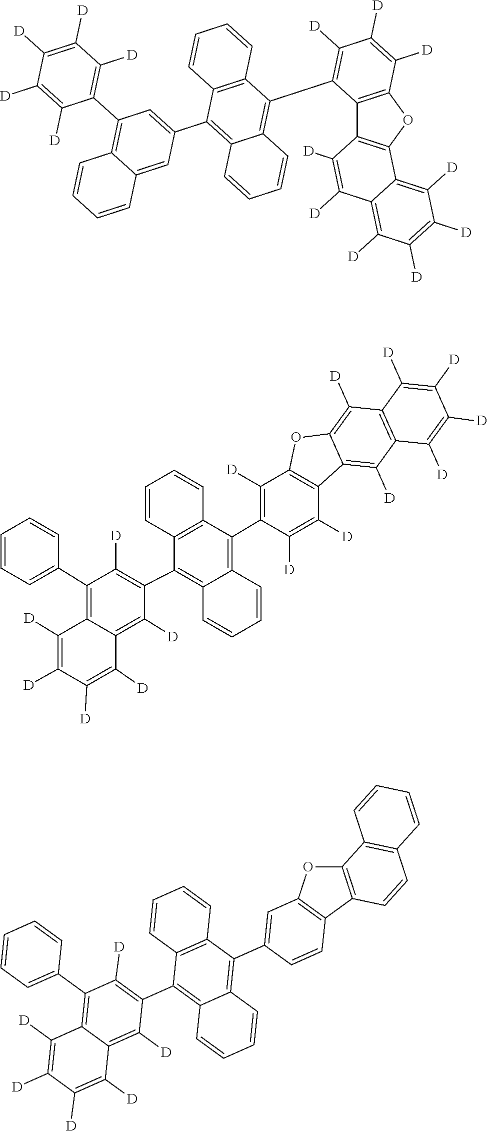 [SMILES: [2H]c1c([2H])c([2H])c(-c2cc(-c3c4ccccc4c(-c4c([2H])c([2H])c([2H])c5oc6c7c([2H])c([2H])c([2H])c([2H])c7c([2H])c([2H])c6c45)c4ccccc34)cc3ccccc23)c([2H])c1[2H] ; [2H]c1c([2H])c([2H])c2c(-c3ccccc3)c([2H])c(-c3c4ccccc4c(-c4c([2H])c([2H])c5c(oc6c([2H])c7c([2H])c([2H])c([2H])c([2H])c7c([2H])c65)c4[2H])c4ccccc34)c([2H])c2c1[2H] ; [2H]c1c([2H])c([2H])c2c(-c3ccccc3)c([2H])c(-c3c4ccccc4c(-c4ccc5c(c4)oc4c6ccccc6ccc54)c4ccccc34)c([2H])c2c1[2H]]

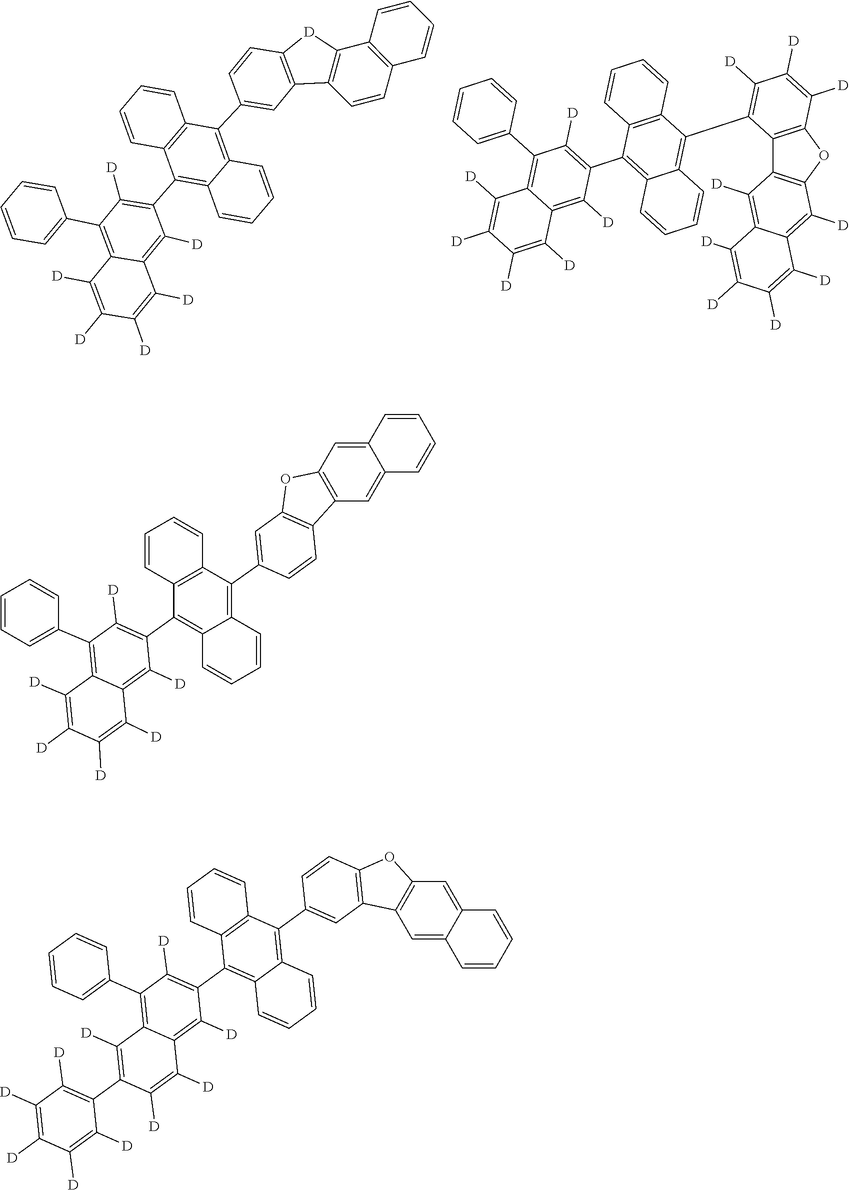

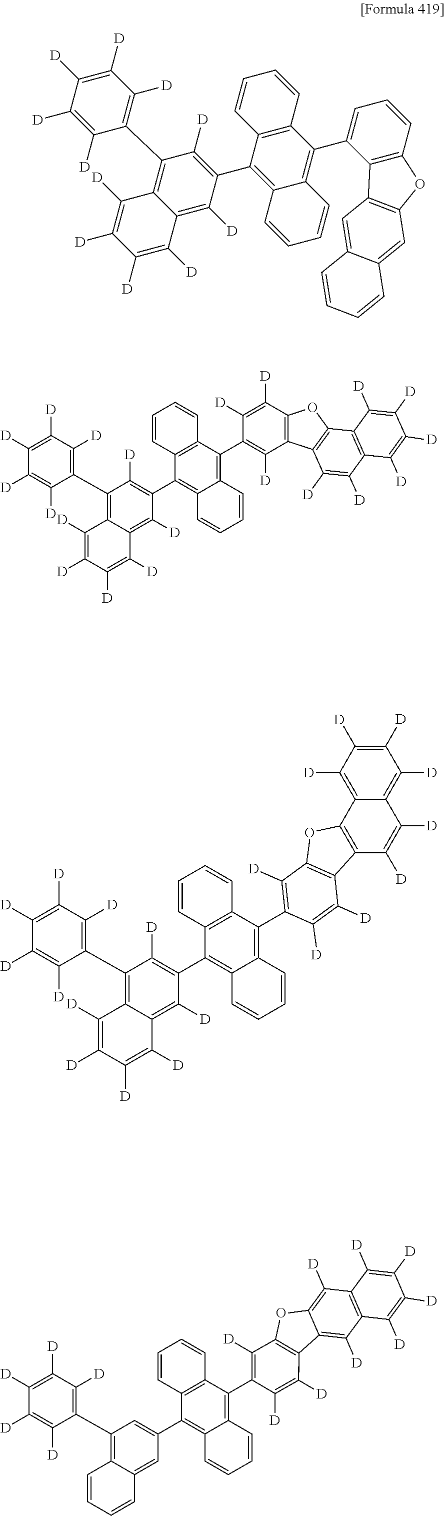

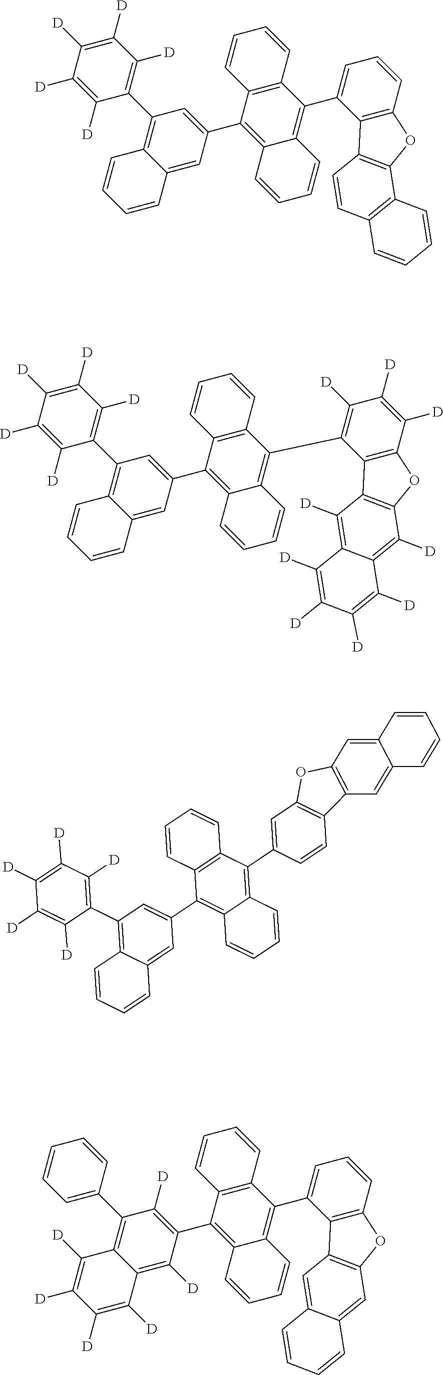 [SMILES: [2H]c1c([2H])c([2H])c(-c2cc(-c3c4ccccc4c(-c4cccc5oc6c7ccccc7ccc6c45)c4ccccc34)cc3ccccc23)c([2H])c1[2H] ; [2H]c1c([2H])c([2H])c(-c2cc(-c3c4ccccc4c(-c4c([2H])c([2H])c([2H])c5oc6c([2H])c7c([2H])c([2H])c([2H])c([2H])c7c([2H])c6c45)c4ccccc34)cc3ccccc23)c([2H])c1[2H] ; [2H]c1c([2H])c([2H])c(-c2cc(-c3c4ccccc4c(-c4ccc5c(c4)oc4cc6ccccc6cc45)c4ccccc34)cc3ccccc23)c([2H])c1[2H] ; [2H]c1c([2H])c([2H])c2c(-c3ccccc3)c([2H])c(-c3c4ccccc4c(-c4cccc5oc6cc7ccccc7cc6c45)c4ccccc34)c([2H])c2c1[2H]]

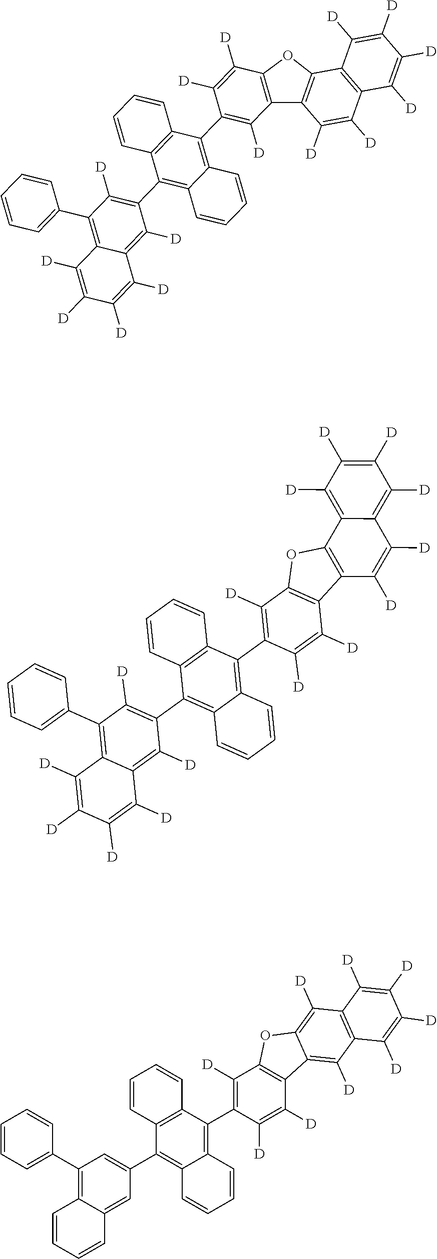 [SMILES: [2H]c1c(-c2c3ccccc3c(-c3c([2H])c(-c4ccccc4)c4c([2H])c([2H])c([2H])c([2H])c4c3[2H])c3ccccc23)c([2H])c2c(oc3c4c([2H])c([2H])c([2H])c([2H])c4c([2H])c([2H])c32)c1[2H] ; [2H]c1c([2H])c([2H])c2c(-c3ccccc3)c([2H])c(-c3c4ccccc4c(-c4c([2H])c([2H])c5c(oc6c7c([2H])c([2H])c([2H])c([2H])c7c([2H])c([2H])c65)c4[2H])c4ccccc34)c([2H])c2c1[2H] ; [2H]c1c([2H])c([2H])c2c([2H])c3c(oc4c([2H])c(-c5c6ccccc6c(-c6cc(-c7ccccc7)c7ccccc7c6)c6ccccc56)c([2H])c([2H])c43)c([2H])c2c1[2H]]

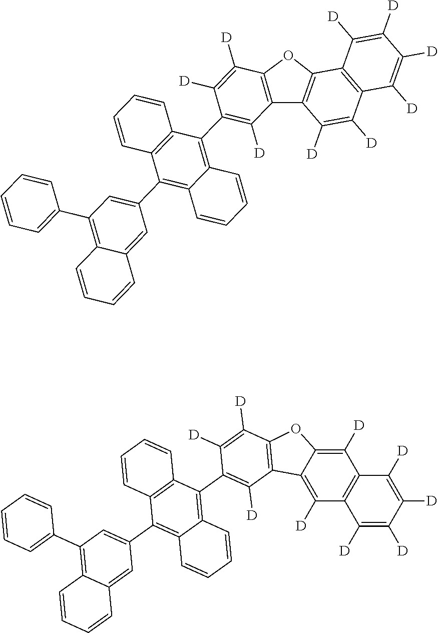 [SMILES: [2H]c1c(-c2c3ccccc3c(-c3cc(-c4ccccc4)c4ccccc4c3)c3ccccc23)c([2H])c2c(oc3c4c([2H])c([2H])c([2H])c([2H])c4c([2H])c([2H])c32)c1[2H] ; [2H]c1c(-c2c3ccccc3c(-c3cc(-c4ccccc4)c4ccccc4c3)c3ccccc23)c([2H])c2c(oc3c([2H])c4c([2H])c([2H])c([2H])c([2H])c4c([2H])c32)c1[2H]]

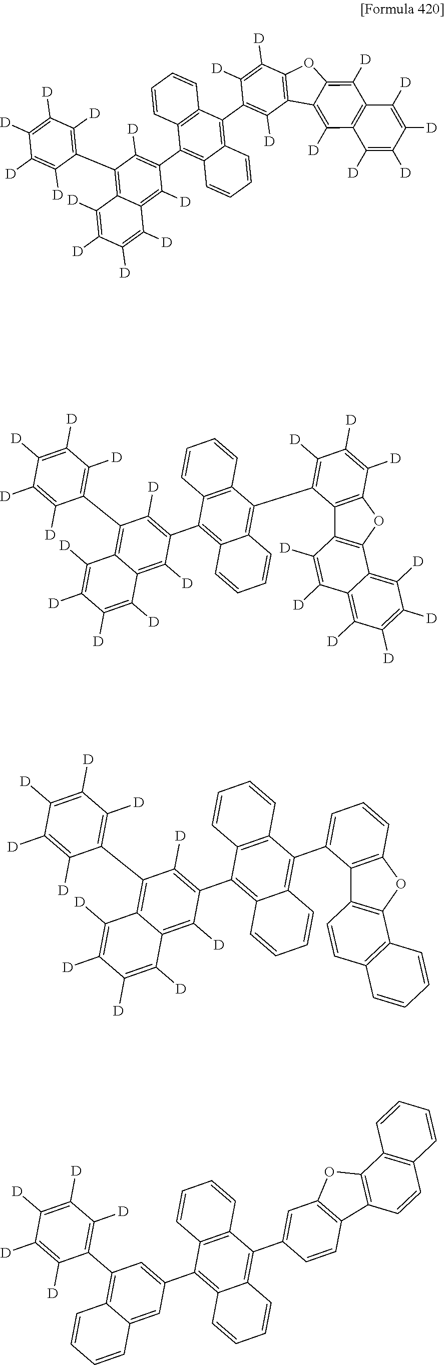

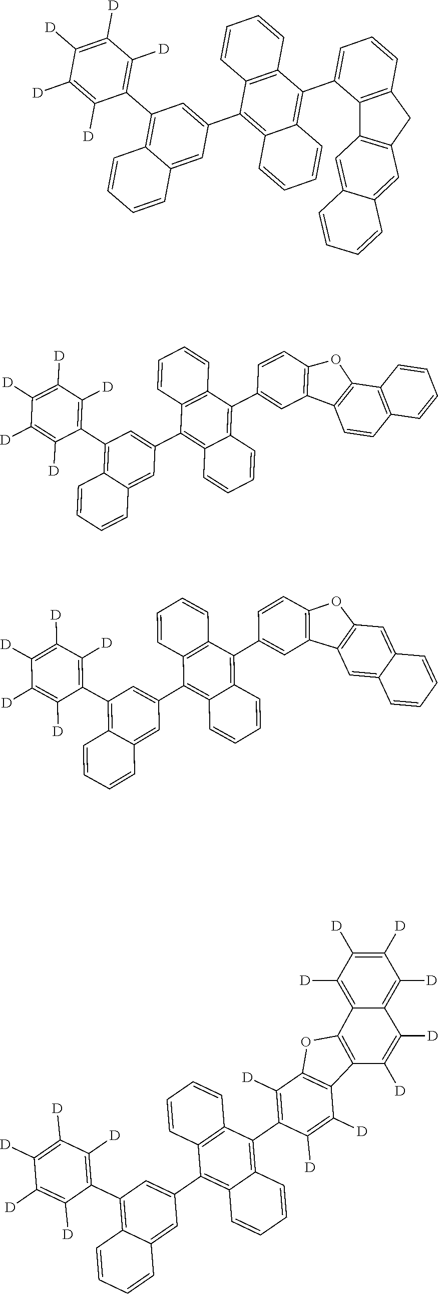 [SMILES: [2H]c1c([2H])c([2H])c(-c2cc(-c3c4ccccc4c(-c4cccc5c4-c4cc6ccccc6cc4C5)c4ccccc34)cc3ccccc23)c([2H])c1[2H] ; [2H]c1c([2H])c([2H])c(-c2cc(-c3c4ccccc4c(-c4ccc5oc6c7ccccc7ccc6c5c4)c4ccccc34)cc3ccccc23)c([2H])c1[2H] ; [2H]c1c([2H])c([2H])c(-c2cc(-c3c4ccccc4c(-c4ccc5oc6cc7ccccc7cc6c5c4)c4ccccc34)cc3ccccc23)c([2H])c1[2H] ; [2H]c1c([2H])c([2H])c(-c2cc(-c3c4ccccc4c(-c4c([2H])c([2H])c5c(oc6c7c([2H])c([2H])c([2H])c([2H])c7c([2H])c([2H])c65)c4[2H])c4ccccc34)cc3ccccc23)c([2H])c1[2H]]

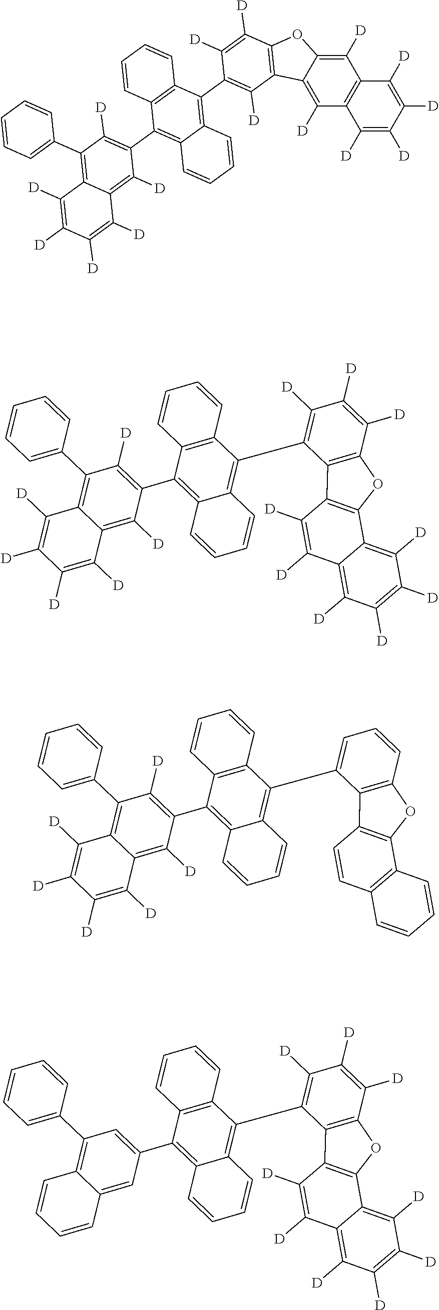

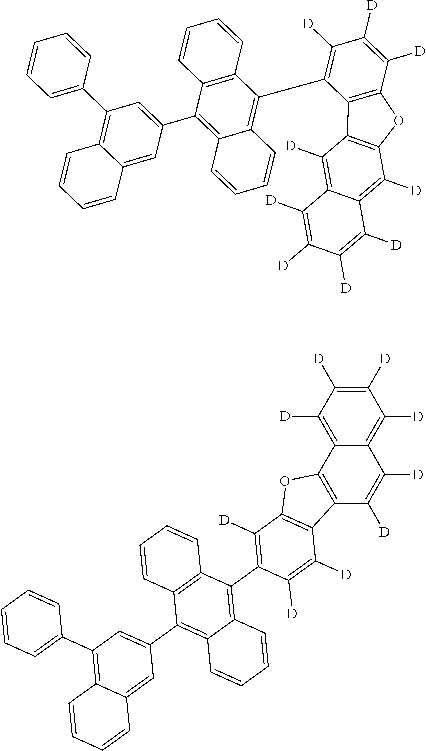 [SMILES: [2H]c1c([2H])c(-c2c3ccccc3c(-c3cc(-c4ccccc4)c4ccccc4c3)c3ccccc23)c2c(oc3c([2H])c4c([2H])c([2H])c([2H])c([2H])c4c([2H])c32)c1[2H] ; [2H]c1c([2H])c([2H])c2c(c1[2H])c([2H])c([2H])c1c2oc2c([2H])c(-c3c4ccccc4c(-c4cc(-c5ccccc5)c5ccccc5c4)c4ccccc34)c([2H])c([2H])c21]

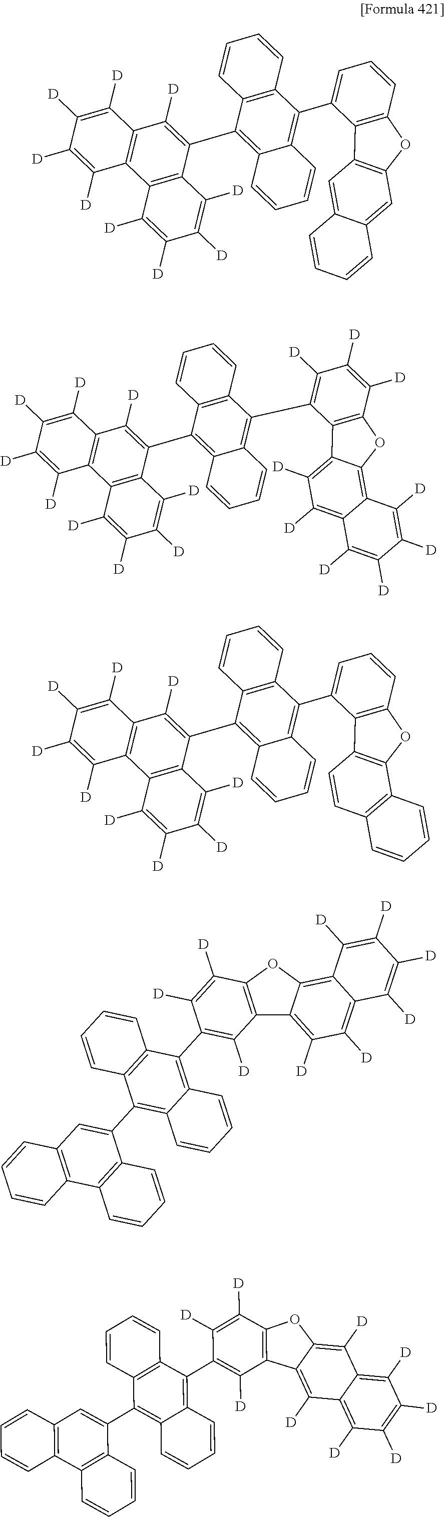

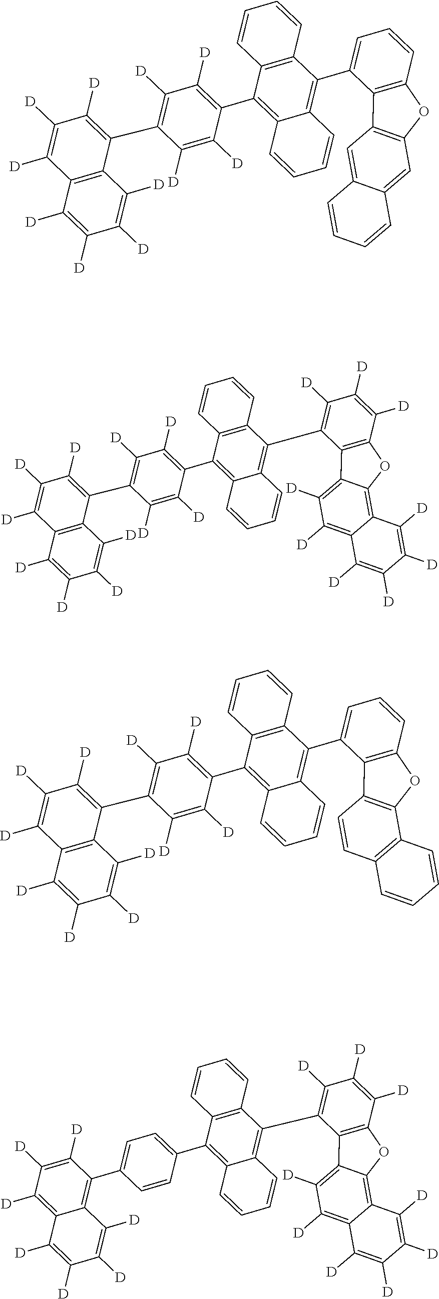

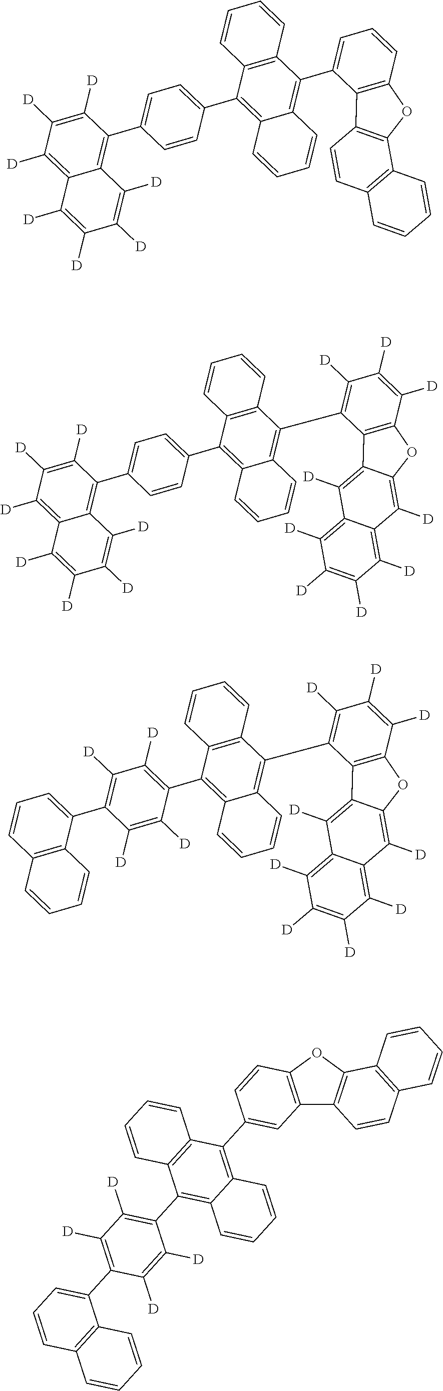

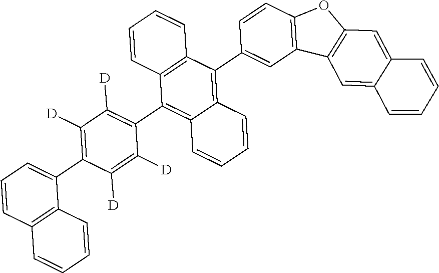 [SMILES: [2H]c1c([2H])c(-c2c3ccccc3c(-c3ccc4oc5cc6ccccc6cc5c4c3)c3ccccc23)c([2H])c([2H])c1-c1cccc2ccccc12]

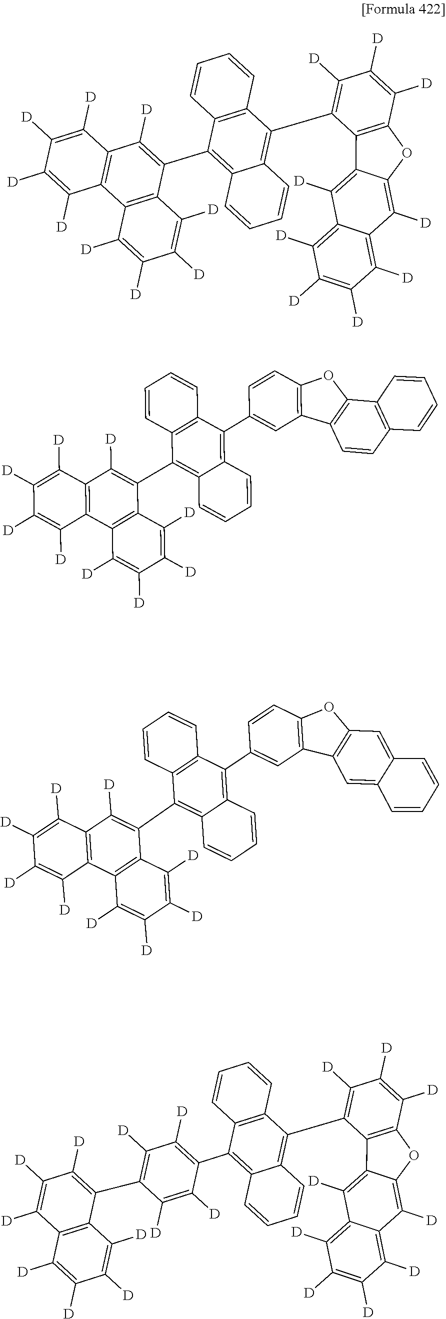

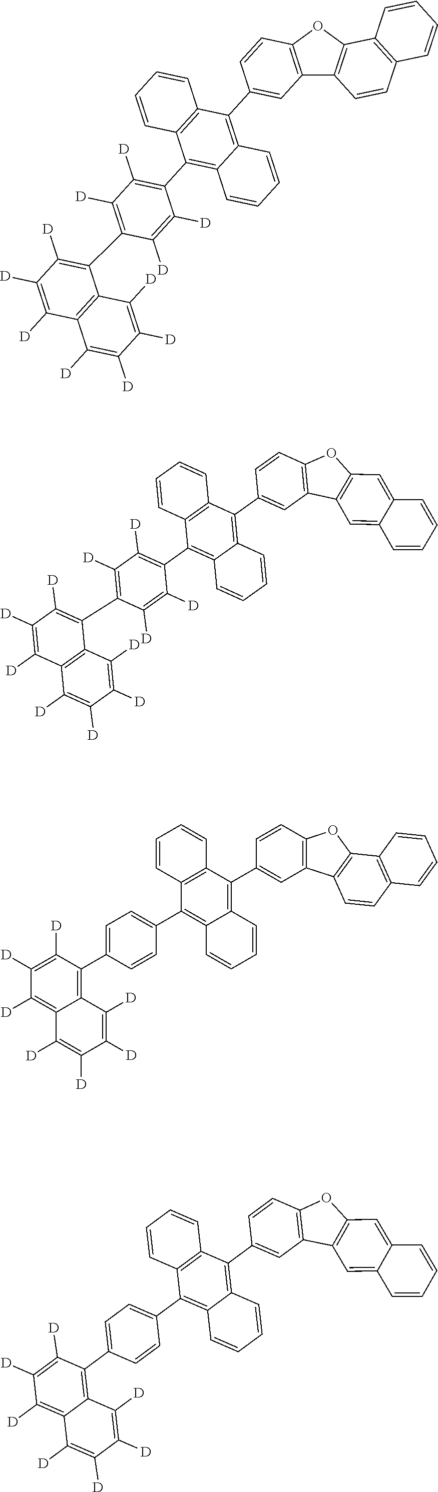

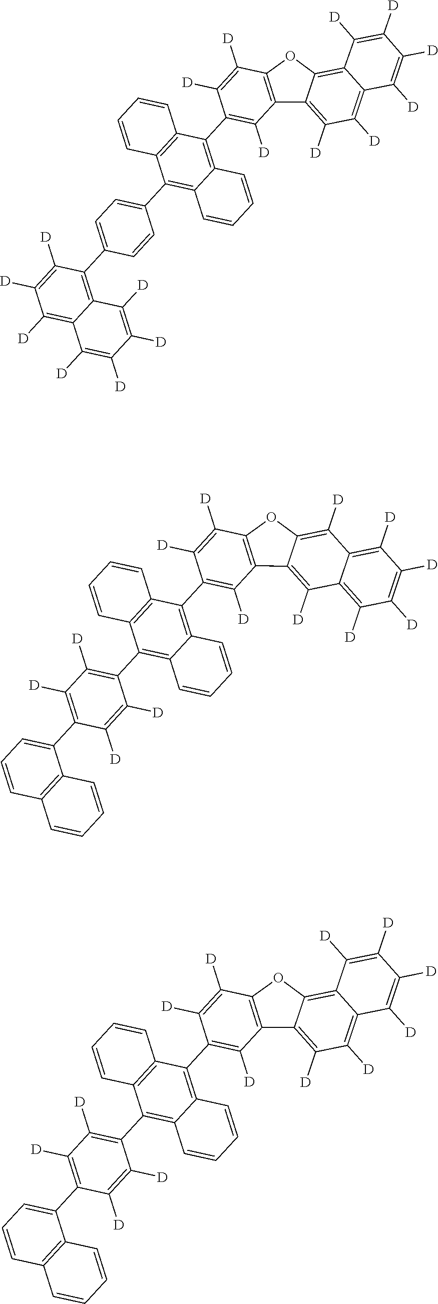 [SMILES: [2H]c1c(-c2c3ccccc3c(-c3ccc(-c4c([2H])c([2H])c([2H])c5c([2H])c([2H])c([2H])c([2H])c45)cc3)c3ccccc23)c([2H])c2c(oc3c4c([2H])c([2H])c([2H])c([2H])c4c([2H])c([2H])c32)c1[2H] ; [2H]c1c([2H])c(-c2c3ccccc3c(-c3c([2H])c([2H])c4oc5c([2H])c6c([2H])c([2H])c([2H])c([2H])c6c([2H])c5c4c3[2H])c3ccccc23)c([2H])c([2H])c1-c1cccc2ccccc12 ; [2H]c1c([2H])c(-c2c3ccccc3c(-c3c([2H])c([2H])c4oc5c6c([2H])c([2H])c([2H])c([2H])c6c([2H])c([2H])c5c4c3[2H])c3ccccc23)c([2H])c([2H])c1-c1cccc2ccccc12]

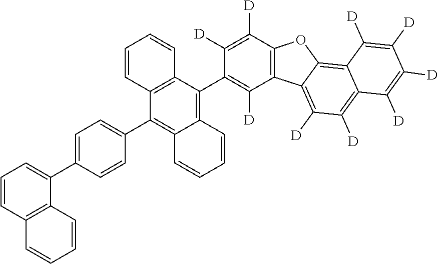 [SMILES: [2H]c1c(-c2c3ccccc3c(-c3ccc(-c4cccc5ccccc45)cc3)c3ccccc23)c([2H])c2c(oc3c4c([2H])c([2H])c([2H])c([2H])c4c([2H])c([2H])c32)c1[2H]]

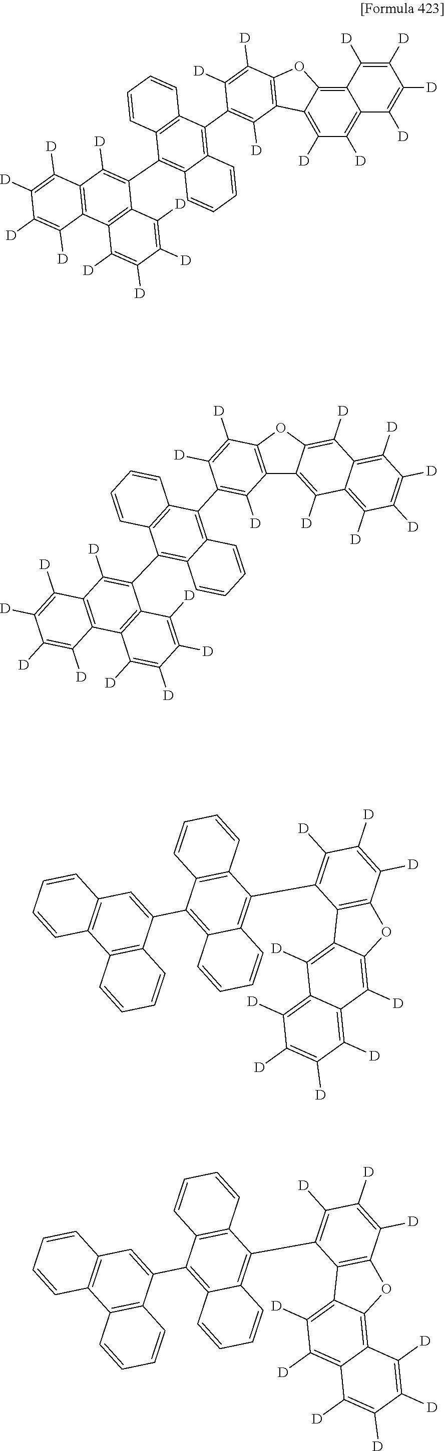

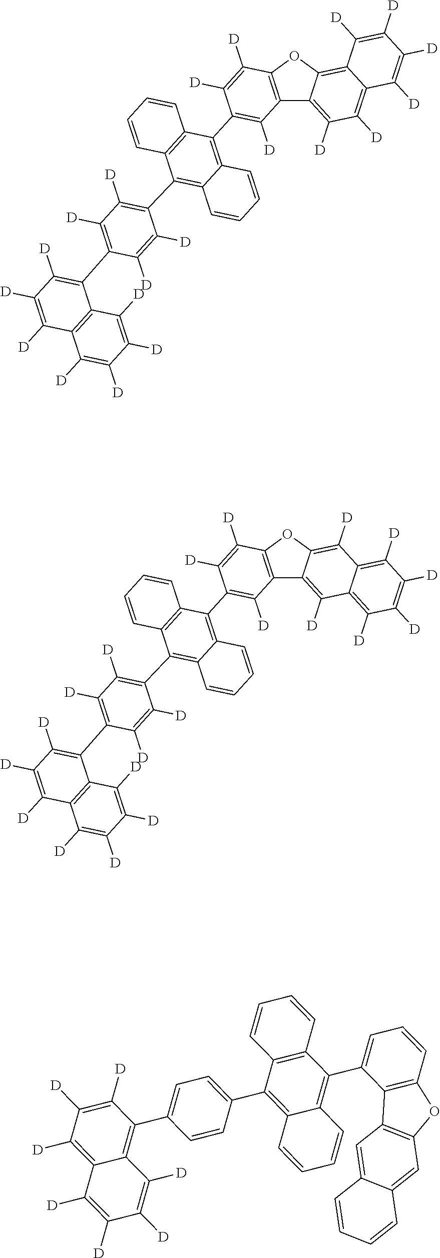 [SMILES: [2H]c1c([2H])c(-c2c([2H])c([2H])c([2H])c3c([2H])c([2H])c([2H])c([2H])c23)c([2H])c([2H])c1-c1c2ccccc2c(-c2c([2H])c([2H])c3oc4c5c([2H])c([2H])c([2H])c([2H])c5c([2H])c([2H])c4c3c2[2H])c2ccccc12 ; [2H]c1c([2H])c(-c2c([2H])c([2H])c([2H])c3c([2H])c([2H])c([2H])c([2H])c23)c([2H])c([2H])c1-c1c2ccccc2c(-c2c([2H])c([2H])c3oc4c([2H])c5c([2H])c([2H])c([2H])c([2H])c5c([2H])c4c3c2[2H])c2ccccc12 ; [2H]c1c([2H])c([2H])c2c(-c3ccc(-c4c5ccccc5c(-c5cccc6oc7cc8ccccc8cc7c56)c5ccccc45)cc3)c([2H])c([2H])c([2H])c2c1[2H]]

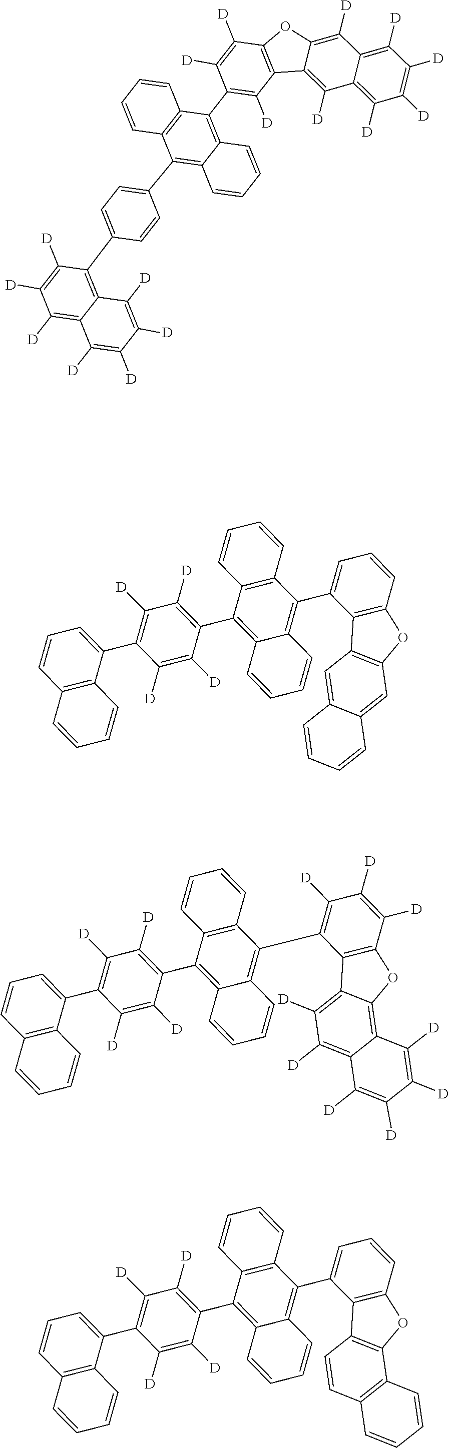

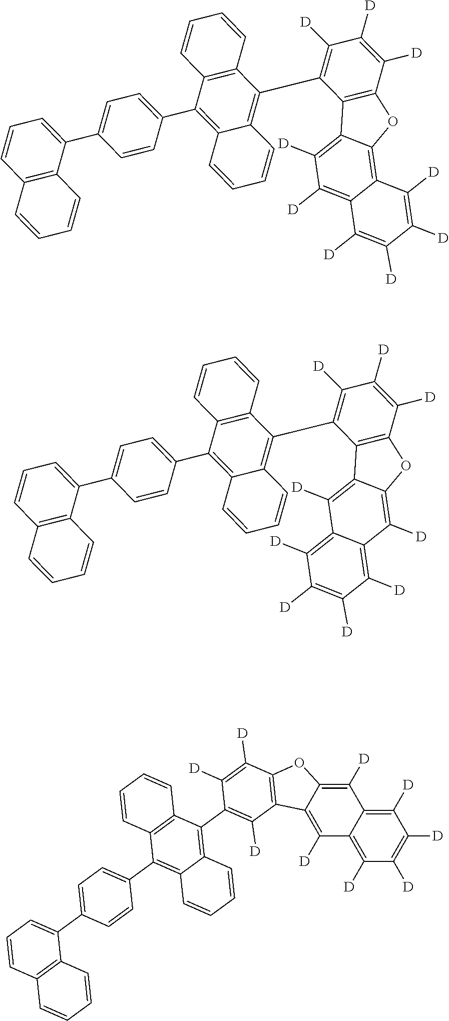 [SMILES: [2H]c1c([2H])c(-c2c3ccccc3c(-c3ccc(-c4cccc5ccccc45)cc3)c3ccccc23)c2c(oc3c4c([2H])c([2H])c([2H])c([2H])c4c([2H])c([2H])c32)c1[2H] ; [2H]c1c([2H])c(-c2c3ccccc3c(-c3ccc(-c4cccc5ccccc45)cc3)c3ccccc23)c2c(oc3c([2H])c4c([2H])c([2H])c([2H])c([2H])c4c([2H])c32)c1[2H] ; [2H]c1c(-c2c3ccccc3c(-c3ccc(-c4cccc5ccccc45)cc3)c3ccccc23)c([2H])c2c(oc3c([2H])c4c([2H])c([2H])c([2H])c([2H])c4c([2H])c32)c1[2H]]

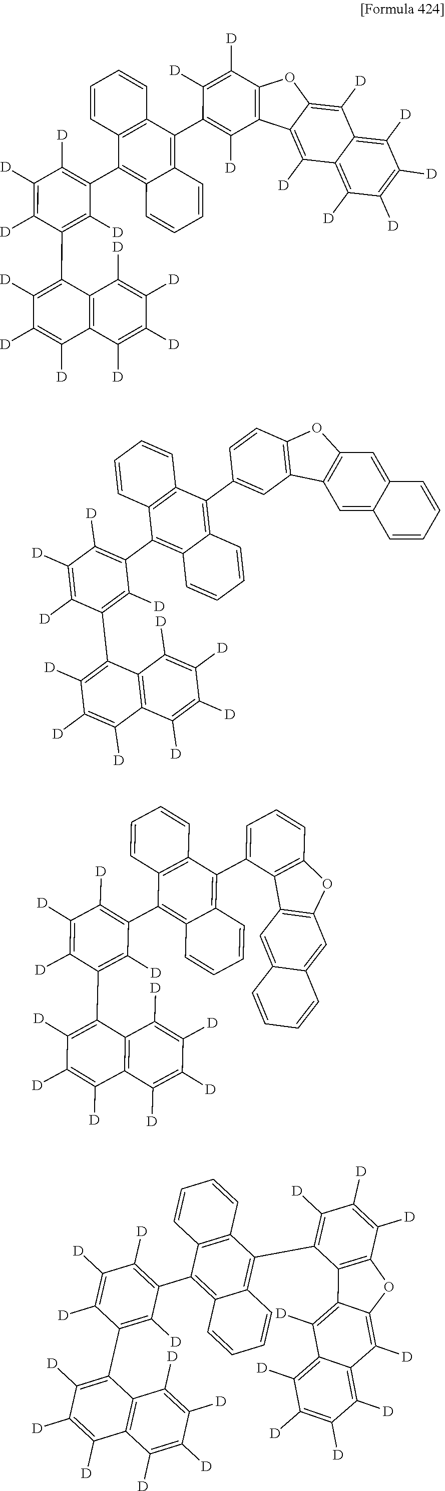

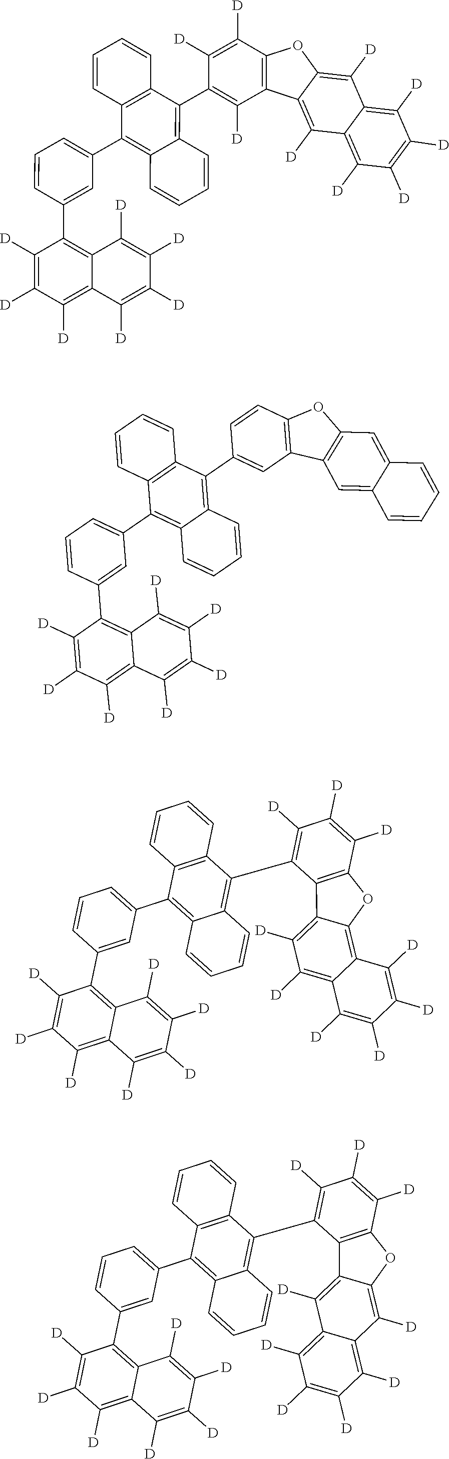

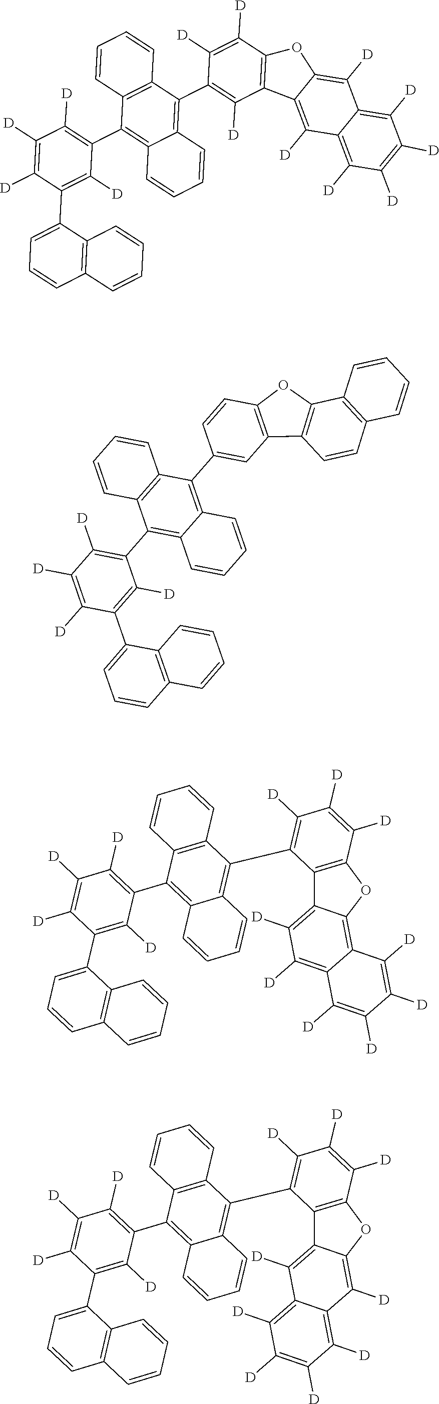

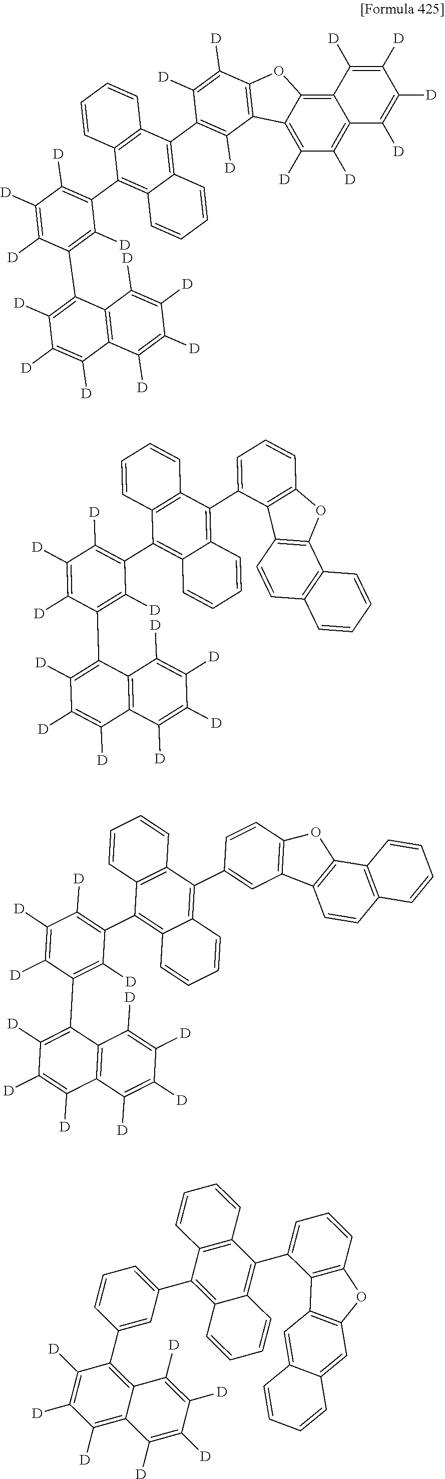

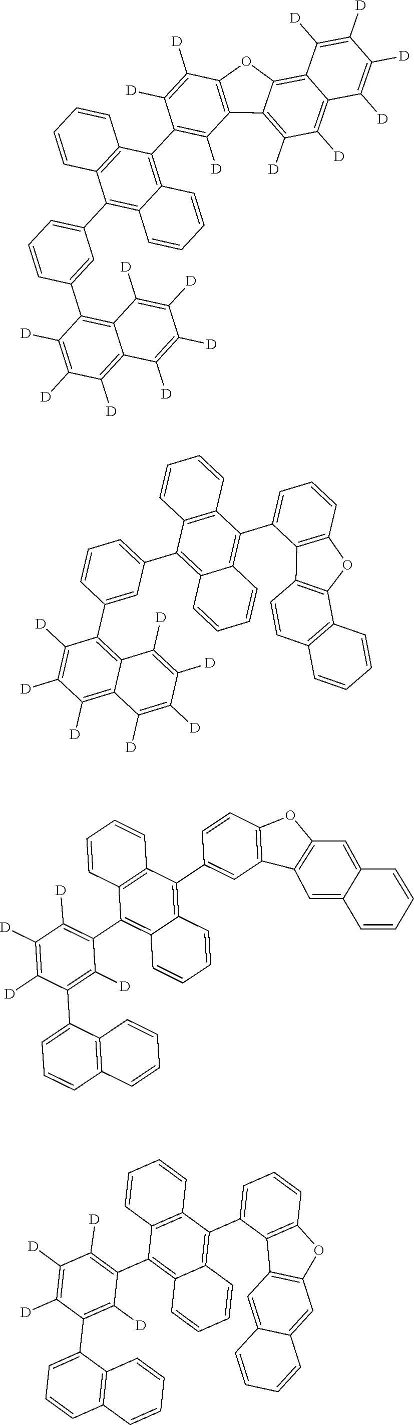 [SMILES: [2H]c1c(-c2c3ccccc3c(-c3cccc(-c4c([2H])c([2H])c([2H])c5c([2H])c([2H])c([2H])c([2H])c45)c3)c3ccccc23)c([2H])c2c(oc3c4c([2H])c([2H])c([2H])c([2H])c4c([2H])c([2H])c32)c1[2H] ; [2H]c1c([2H])c([2H])c2c(-c3cccc(-c4c5ccccc5c(-c5cccc6oc7c8ccccc8ccc7c56)c5ccccc45)c3)c([2H])c([2H])c([2H])c2c1[2H] ; [2H]c1c([2H])c(-c2cccc3ccccc23)c([2H])c(-c2c3ccccc3c(-c3ccc4oc5cc6ccccc6cc5c4c3)c3ccccc23)c1[2H] ; [2H]c1c([2H])c(-c2cccc3ccccc23)c([2H])c(-c2c3ccccc3c(-c3cccc4oc5cc6ccccc6cc5c34)c3ccccc23)c1[2H]]

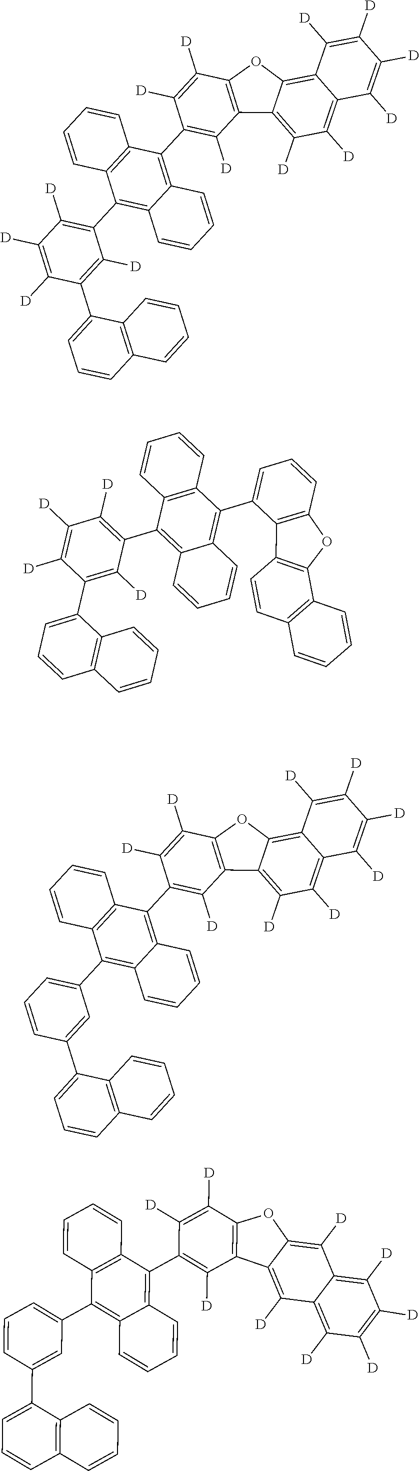

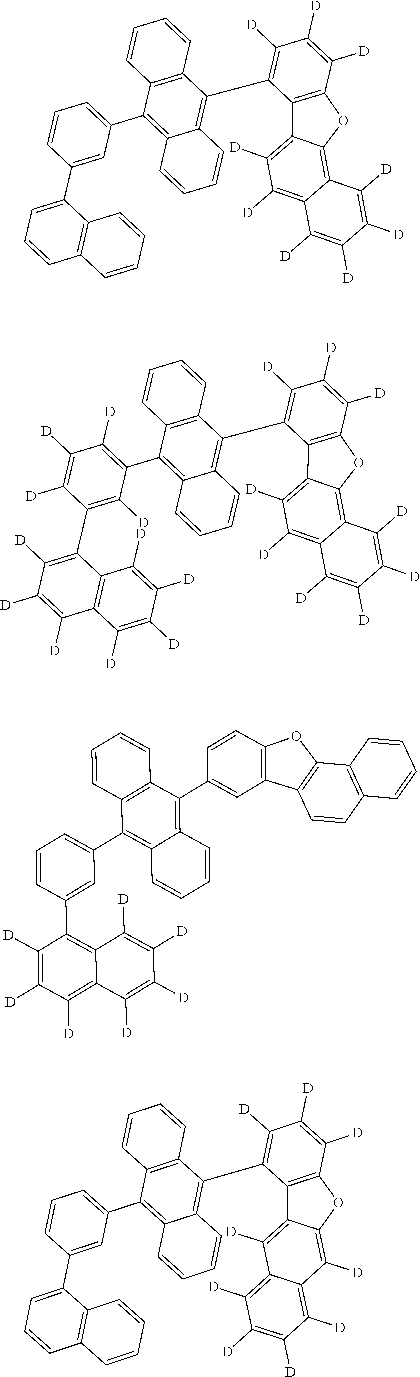

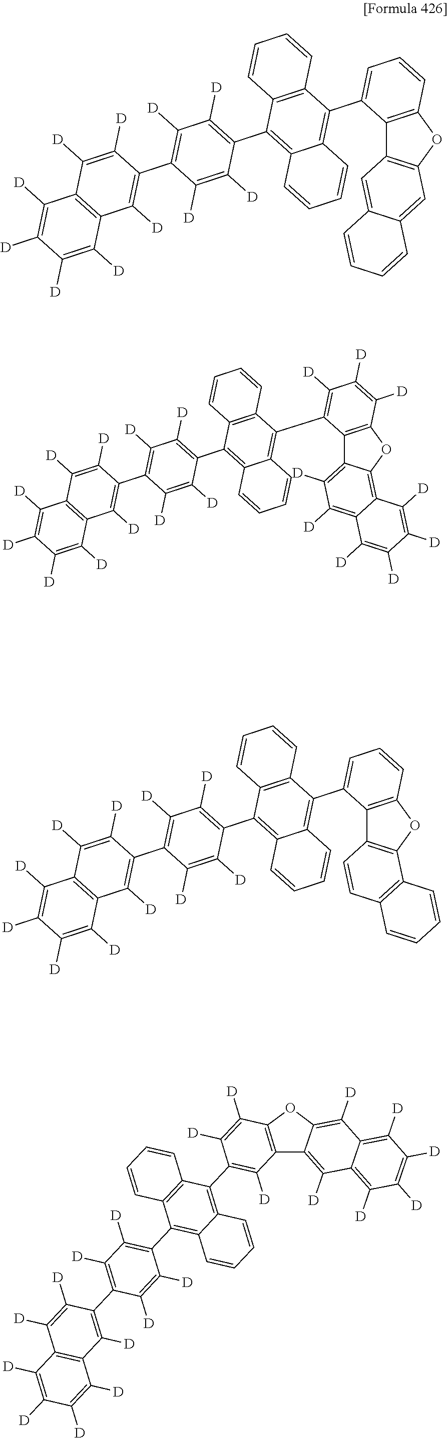

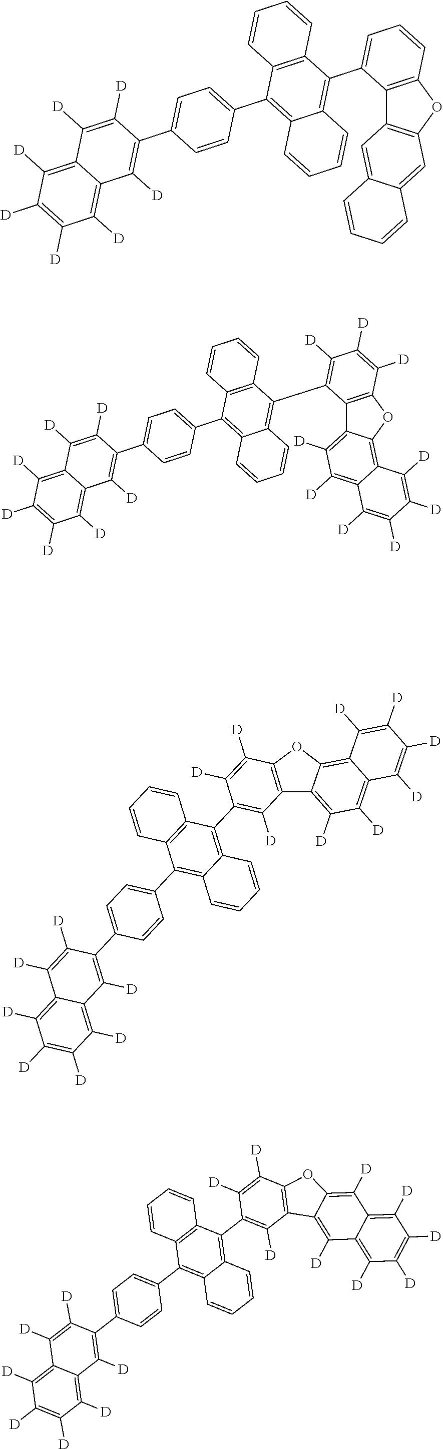

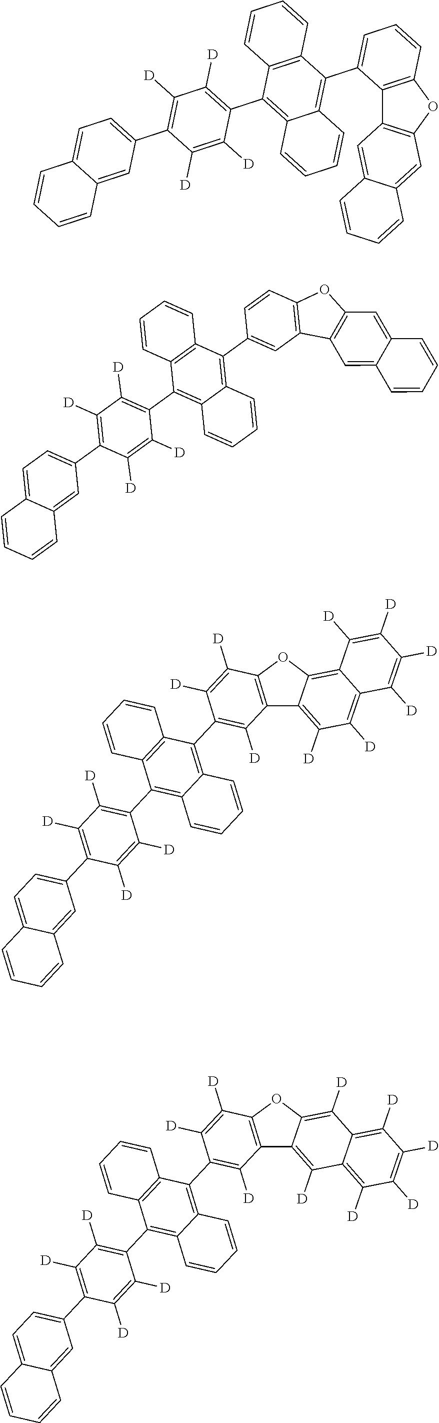 [SMILES: [2H]c1c([2H])c(-c2c3ccccc3c(-c3cccc4oc5cc6ccccc6cc5c34)c3ccccc23)c([2H])c([2H])c1-c1ccc2ccccc2c1 ; [2H]c1c([2H])c(-c2c3ccccc3c(-c3ccc4oc5cc6ccccc6cc5c4c3)c3ccccc23)c([2H])c([2H])c1-c1ccc2ccccc2c1 ; [2H]c1c([2H])c(-c2c3ccccc3c(-c3c([2H])c([2H])c4oc5c6c([2H])c([2H])c([2H])c([2H])c6c([2H])c([2H])c5c4c3[2H])c3ccccc23)c([2H])c([2H])c1-c1ccc2ccccc2c1 ; [2H]c1c([2H])c(-c2c3ccccc3c(-c3c([2H])c([2H])c4oc5c([2H])c6c([2H])c([2H])c([2H])c([2H])c6c([2H])c5c4c3[2H])c3ccccc23)c([2H])c([2H])c1-c1ccc2ccccc2c1]

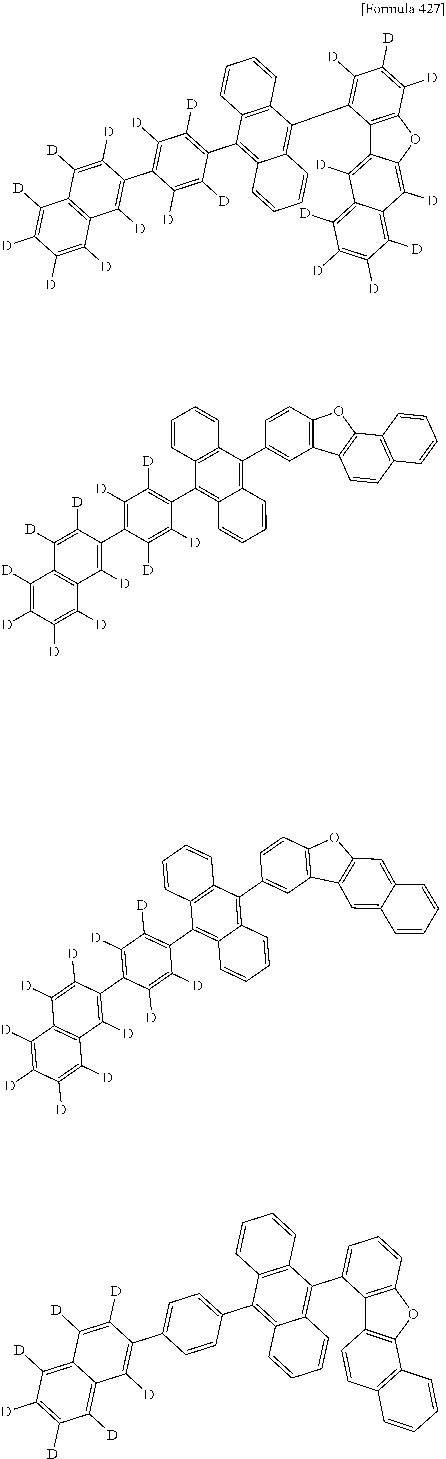

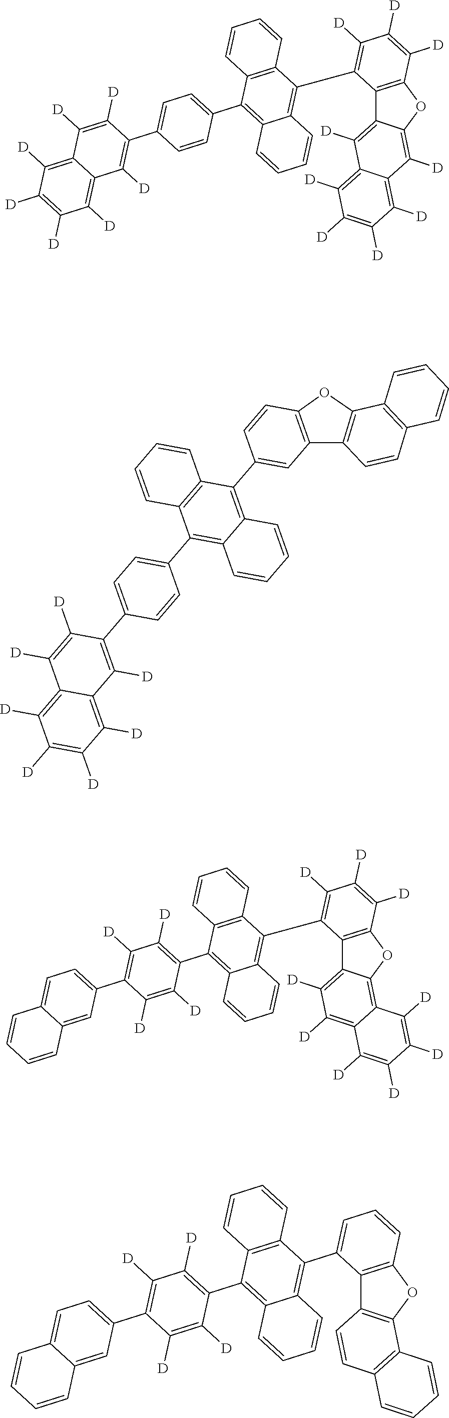

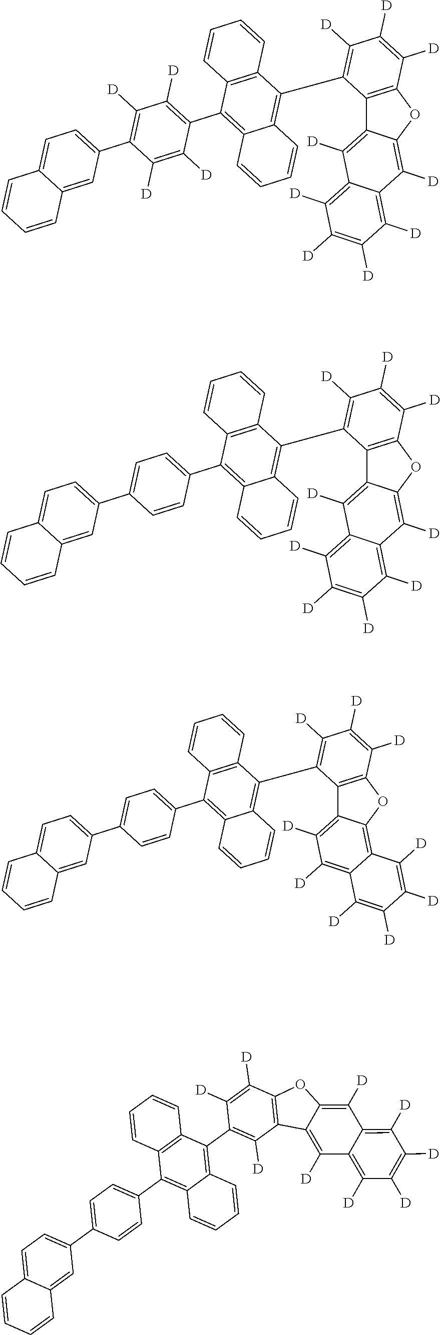

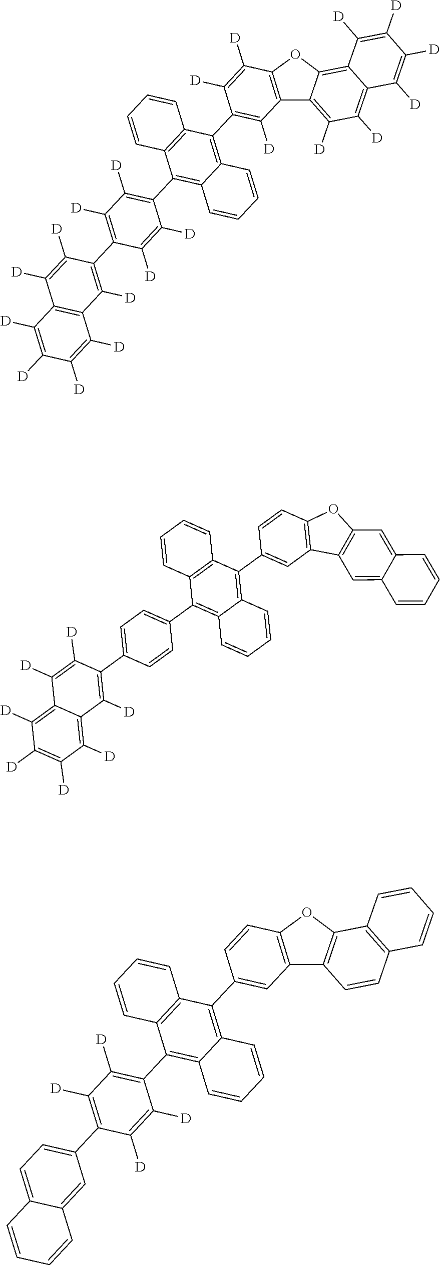 [SMILES: [2H]c1c([2H])c(-c2c3ccccc3c(-c3c([2H])c([2H])c4oc5c6c([2H])c([2H])c([2H])c([2H])c6c([2H])c([2H])c5c4c3[2H])c3ccccc23)c([2H])c([2H])c1-c1c([2H])c([2H])c2c([2H])c([2H])c([2H])c([2H])c2c1[2H] ; [2H]c1c([2H])c([2H])c2c([2H])c(-c3ccc(-c4c5ccccc5c(-c5ccc6oc7cc8ccccc8cc7c6c5)c5ccccc45)cc3)c([2H])c([2H])c2c1[2H] ; [2H]c1c([2H])c(-c2c3ccccc3c(-c3ccc4oc5c6ccccc6ccc5c4c3)c3ccccc23)c([2H])c([2H])c1-c1ccc2ccccc2c1]

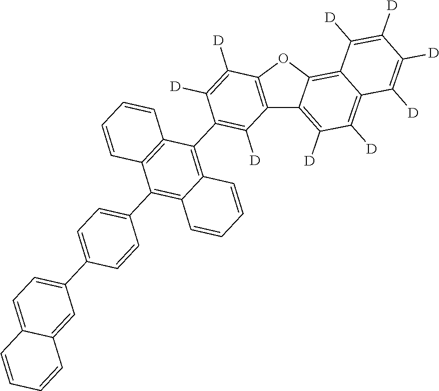 [SMILES: [2H]c1c(-c2c3ccccc3c(-c3ccc(-c4ccc5ccccc5c4)cc3)c3ccccc23)c([2H])c2c(oc3c4c([2H])c([2H])c([2H])c([2H])c4c([2H])c([2H])c32)c1[2H]]

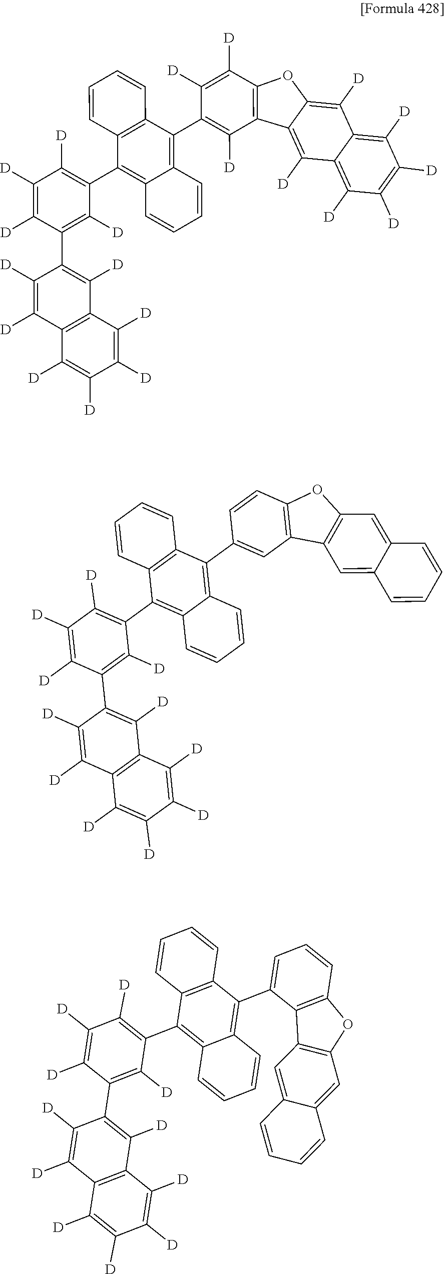 [SMILES: [2H]c1c([2H])c(-c2c([2H])c([2H])c3c([2H])c([2H])c([2H])c([2H])c3c2[2H])c([2H])c(-c2c3ccccc3c(-c3c([2H])c([2H])c4oc5c([2H])c6c([2H])c([2H])c([2H])c([2H])c6c([2H])c5c4c3[2H])c3ccccc23)c1[2H] ; [2H]c1c([2H])c(-c2c([2H])c([2H])c3c([2H])c([2H])c([2H])c([2H])c3c2[2H])c([2H])c(-c2c3ccccc3c(-c3ccc4oc5cc6ccccc6cc5c4c3)c3ccccc23)c1[2H] ; [2H]c1c([2H])c(-c2c([2H])c([2H])c3c([2H])c([2H])c([2H])c([2H])c3c2[2H])c([2H])c(-c2c3ccccc3c(-c3cccc4oc5cc6ccccc6cc5c34)c3ccccc23)c1[2H]]

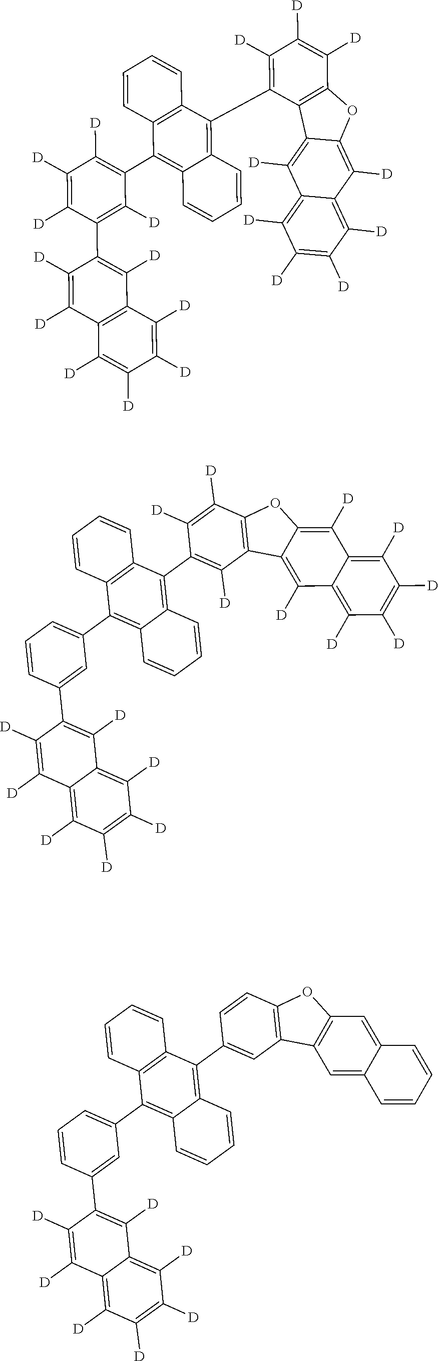 [SMILES: [2H]c1c([2H])c(-c2c([2H])c([2H])c3c([2H])c([2H])c([2H])c([2H])c3c2[2H])c([2H])c(-c2c3ccccc3c(-c3c([2H])c([2H])c([2H])c4oc5c([2H])c6c([2H])c([2H])c([2H])c([2H])c6c([2H])c5c34)c3ccccc23)c1[2H] ; [2H]c1c(-c2c3ccccc3c(-c3cccc(-c4c([2H])c([2H])c5c([2H])c([2H])c([2H])c([2H])c5c4[2H])c3)c3ccccc23)c([2H])c2c(oc3c([2H])c4c([2H])c([2H])c([2H])c([2H])c4c([2H])c32)c1[2H] ; [2H]c1c([2H])c([2H])c2c([2H])c(-c3cccc(-c4c5ccccc5c(-c5ccc6oc7cc8ccccc8cc7c6c5)c5ccccc45)c3)c([2H])c([2H])c2c1[2H]]

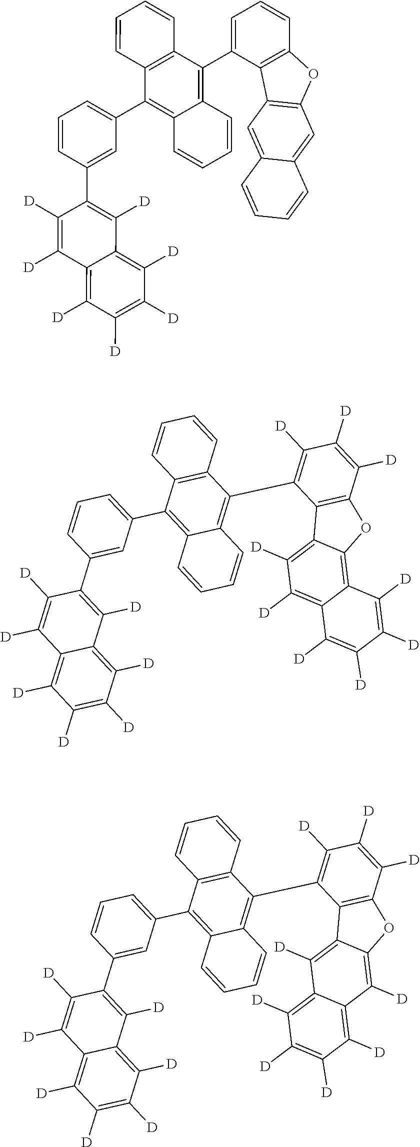 [SMILES: [2H]c1c([2H])c([2H])c2c([2H])c(-c3cccc(-c4c5ccccc5c(-c5cccc6oc7cc8ccccc8cc7c56)c5ccccc45)c3)c([2H])c([2H])c2c1[2H] ; [2H]c1c([2H])c(-c2c3ccccc3c(-c3cccc(-c4c([2H])c([2H])c5c([2H])c([2H])c([2H])c([2H])c5c4[2H])c3)c3ccccc23)c2c(oc3c4c([2H])c([2H])c([2H])c([2H])c4c([2H])c([2H])c32)c1[2H] ; [2H]c1c([2H])c(-c2c3ccccc3c(-c3cccc(-c4c([2H])c([2H])c5c([2H])c([2H])c([2H])c([2H])c5c4[2H])c3)c3ccccc23)c2c(oc3c([2H])c4c([2H])c([2H])c([2H])c([2H])c4c([2H])c32)c1[2H]]

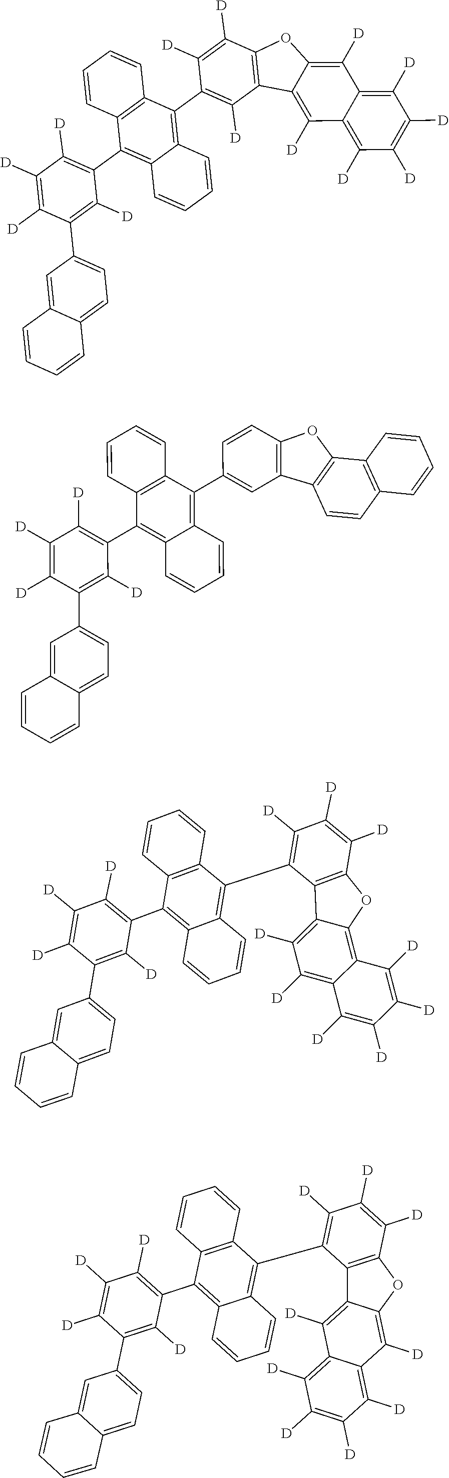

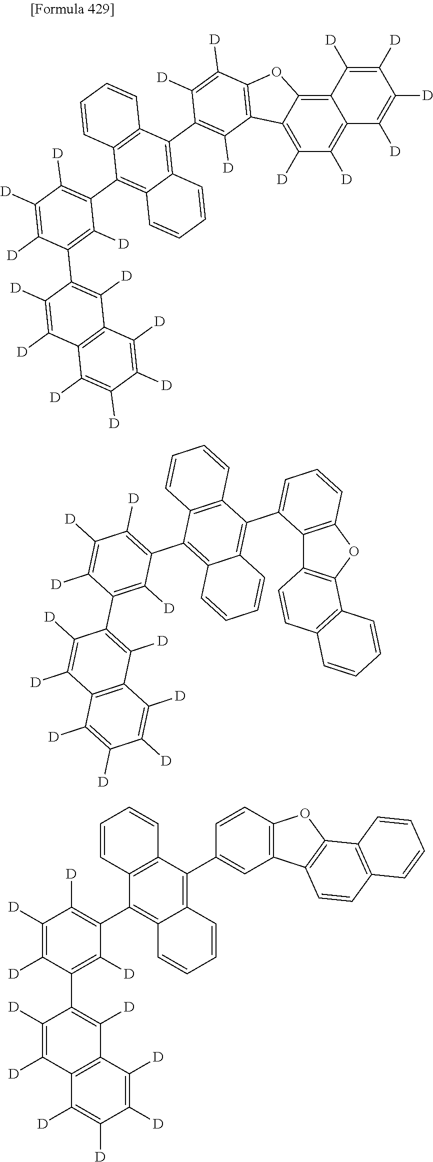

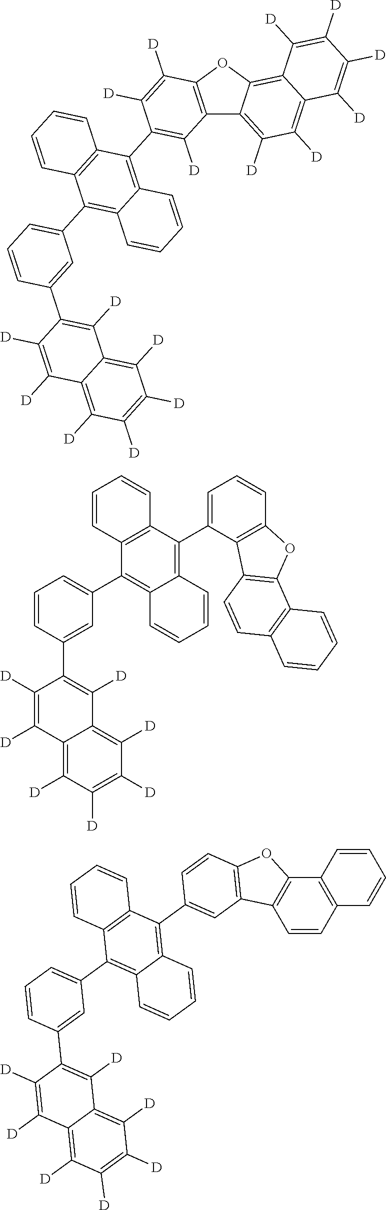

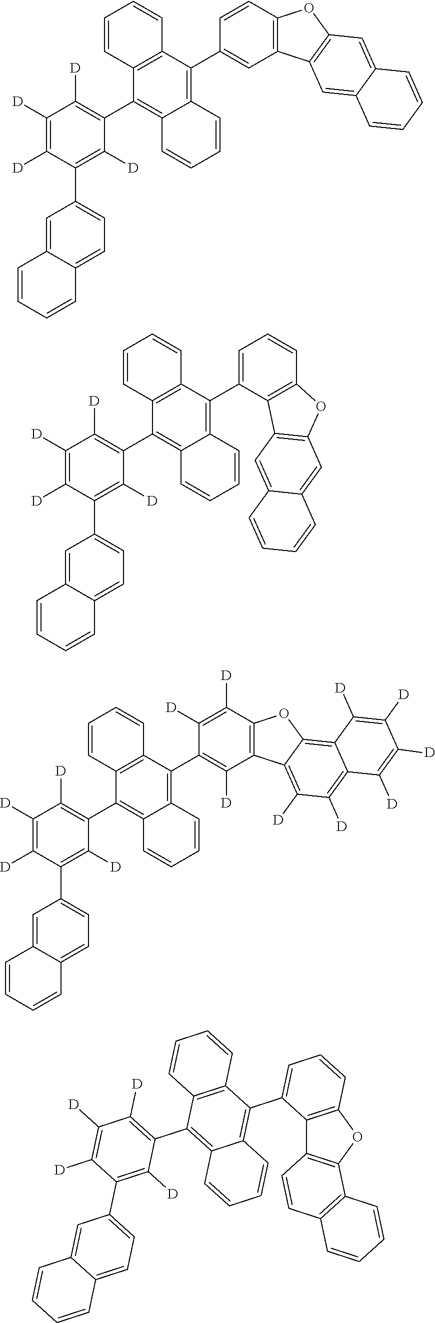 [SMILES: [2H]c1c([2H])c(-c2ccc3ccccc3c2)c([2H])c(-c2c3ccccc3c(-c3ccc4oc5cc6ccccc6cc5c4c3)c3ccccc23)c1[2H] ; [2H]c1c([2H])c(-c2ccc3ccccc3c2)c([2H])c(-c2c3ccccc3c(-c3cccc4oc5cc6ccccc6cc5c34)c3ccccc23)c1[2H] ; [2H]c1c([2H])c(-c2ccc3ccccc3c2)c([2H])c(-c2c3ccccc3c(-c3c([2H])c([2H])c4oc5c6c([2H])c([2H])c([2H])c([2H])c6c([2H])c([2H])c5c4c3[2H])c3ccccc23)c1[2H] ; [2H]c1c([2H])c(-c2ccc3ccccc3c2)c([2H])c(-c2c3ccccc3c(-c3cccc4oc5c6ccccc6ccc5c34)c3ccccc23)c1[2H]]

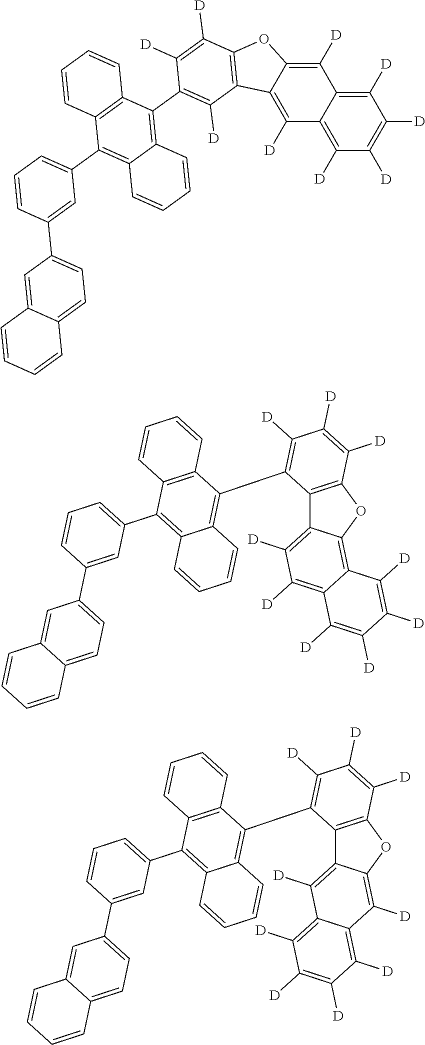 [SMILES: [2H]c1c(-c2c3ccccc3c(-c3cccc(-c4ccc5ccccc5c4)c3)c3ccccc23)c([2H])c2c(oc3c([2H])c4c([2H])c([2H])c([2H])c([2H])c4c([2H])c32)c1[2H] ; [2H]c1c([2H])c(-c2c3ccccc3c(-c3cccc(-c4ccc5ccccc5c4)c3)c3ccccc23)c2c(oc3c4c([2H])c([2H])c([2H])c([2H])c4c([2H])c([2H])c32)c1[2H] ; [2H]c1c([2H])c(-c2c3ccccc3c(-c3cccc(-c4ccc5ccccc5c4)c3)c3ccccc23)c2c(oc3c([2H])c4c([2H])c([2H])c([2H])c([2H])c4c([2H])c32)c1[2H]]

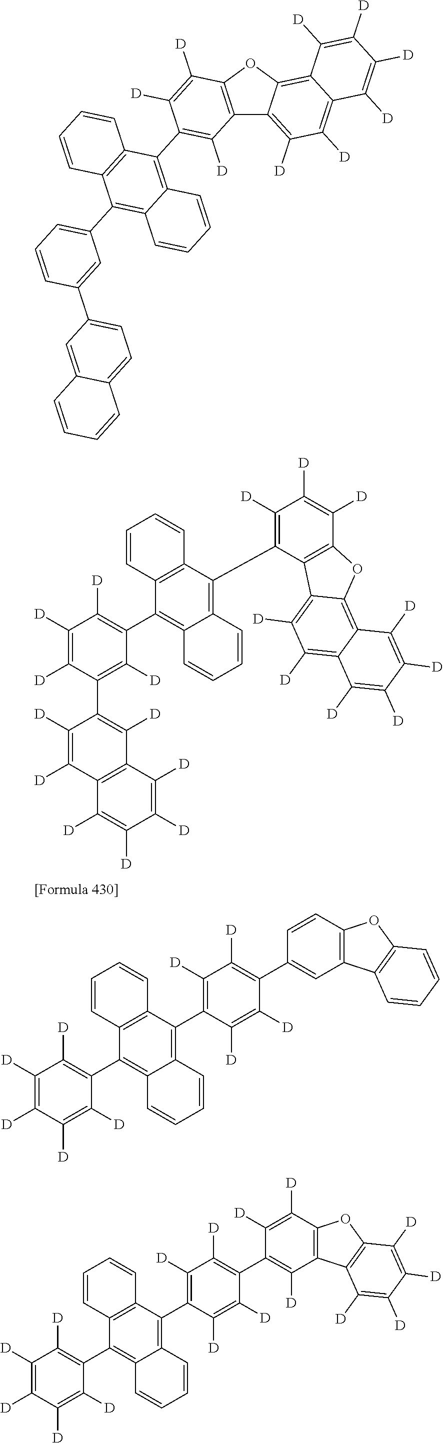

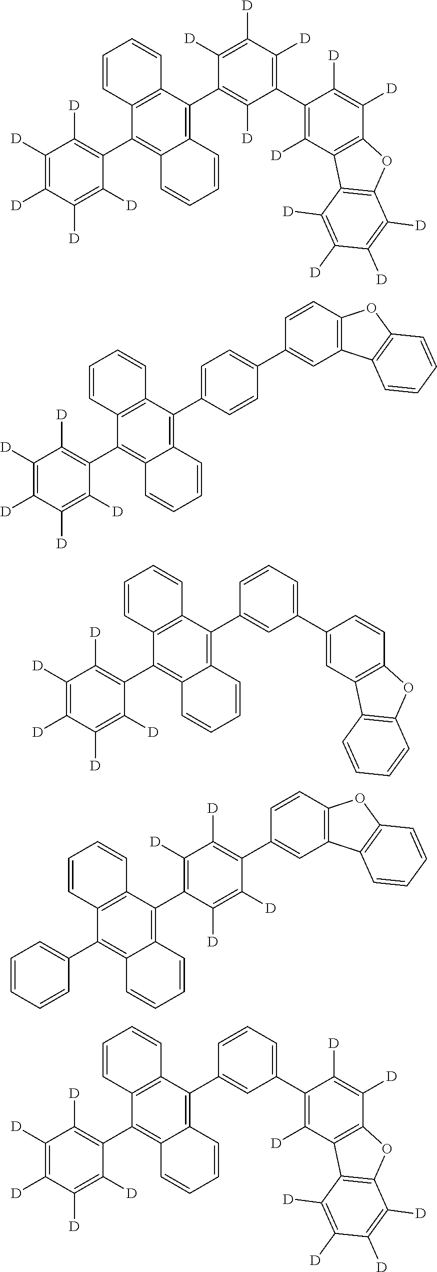

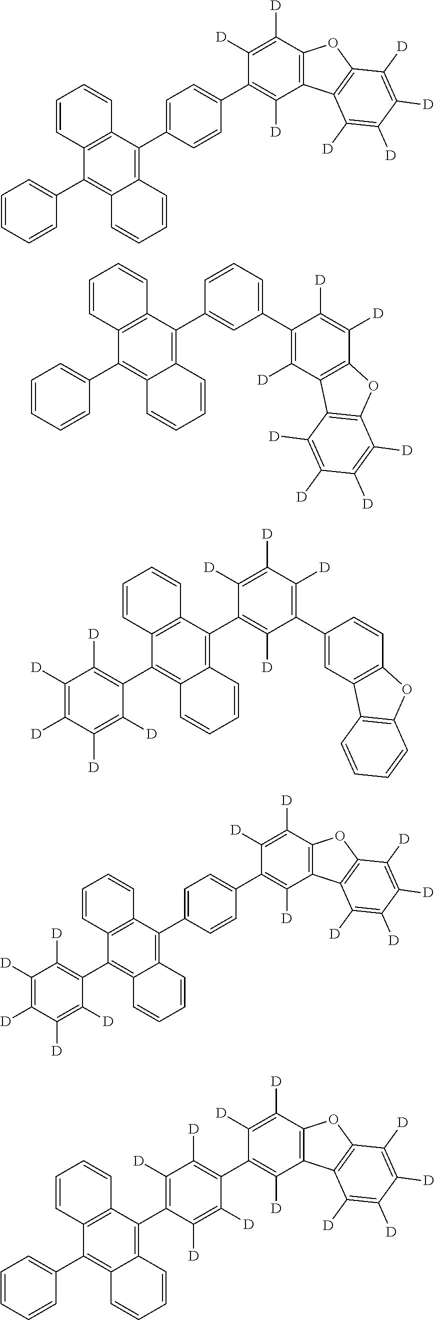

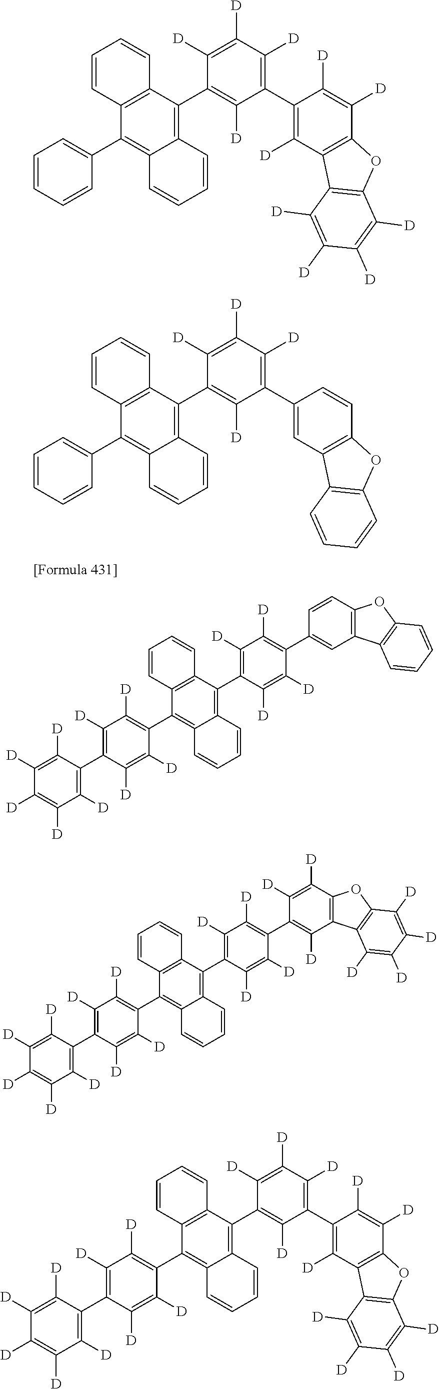

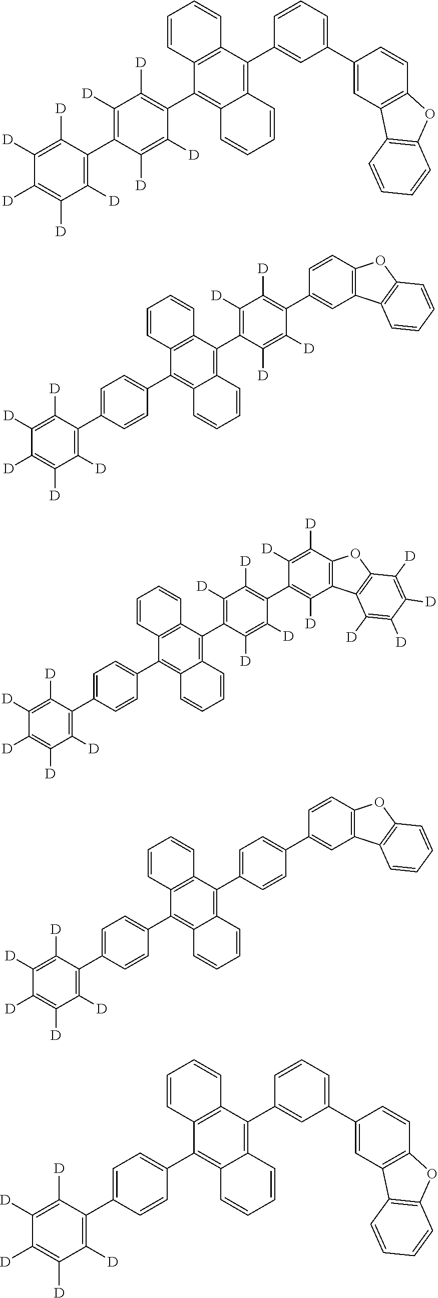

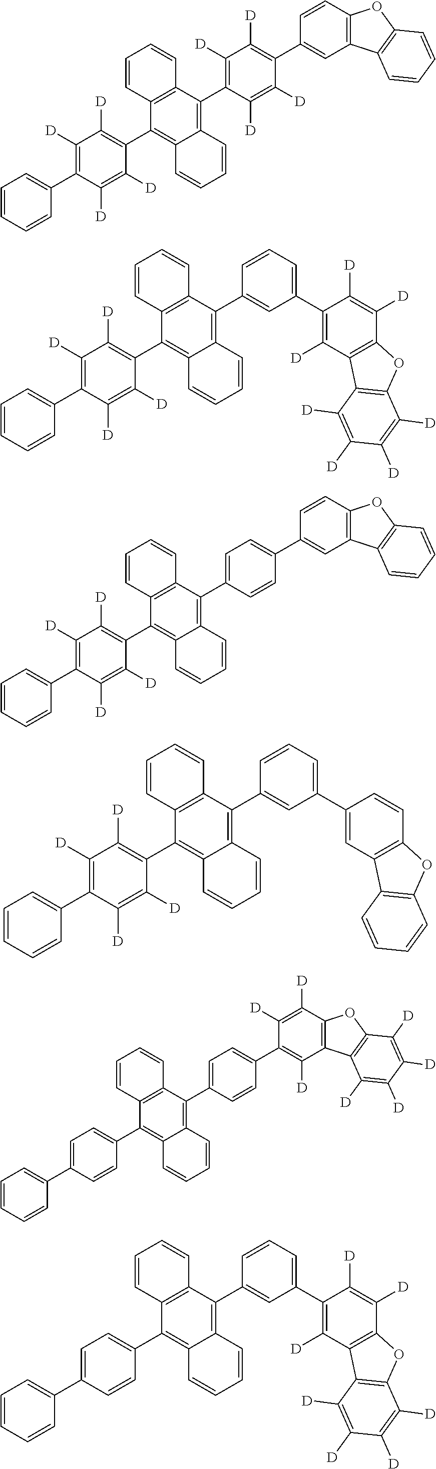

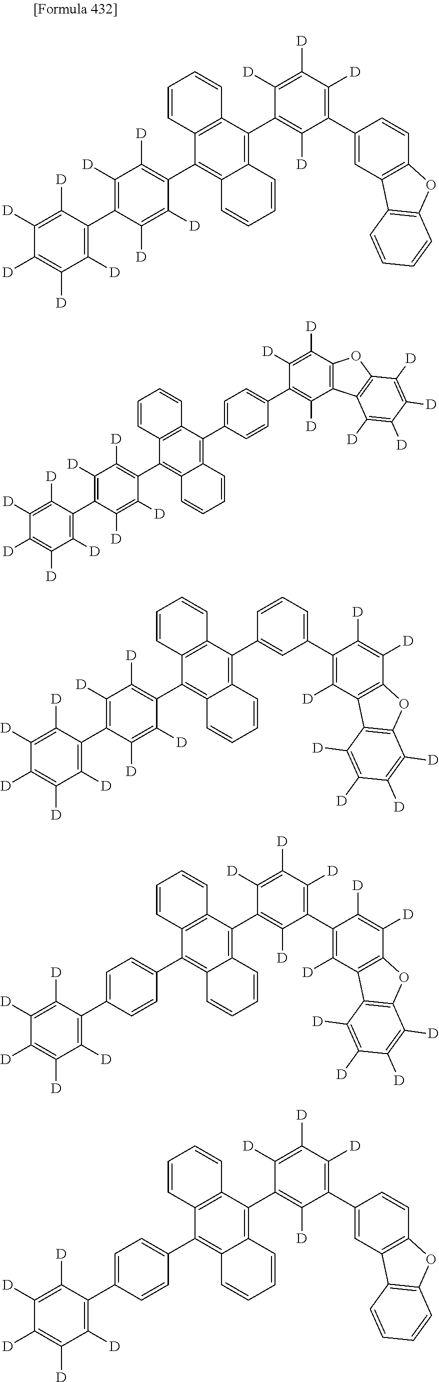

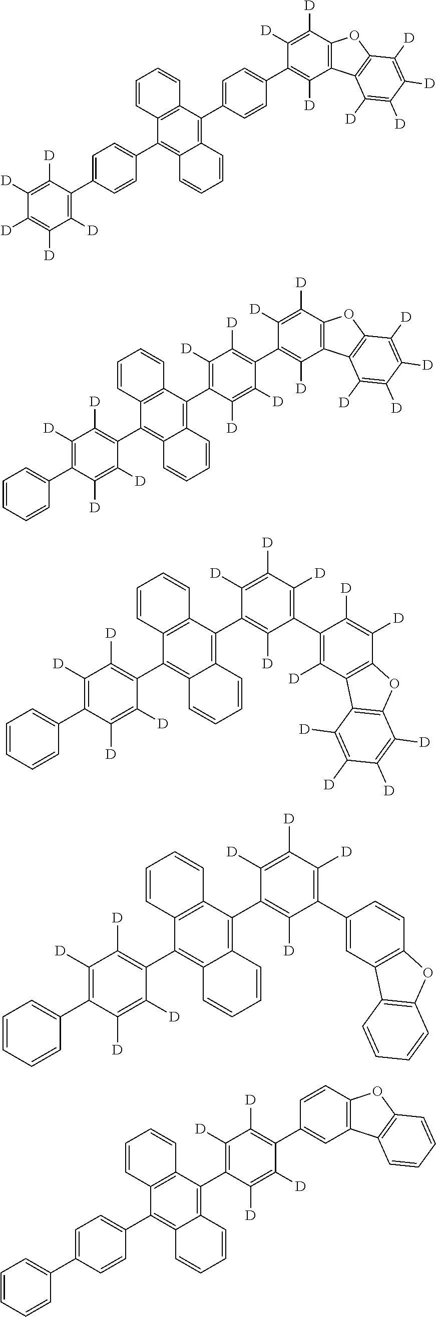

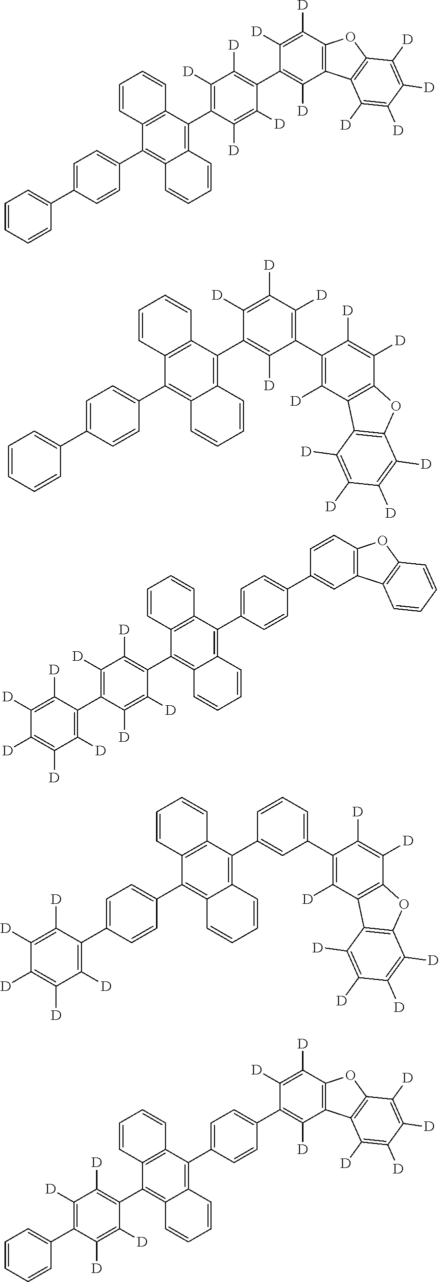

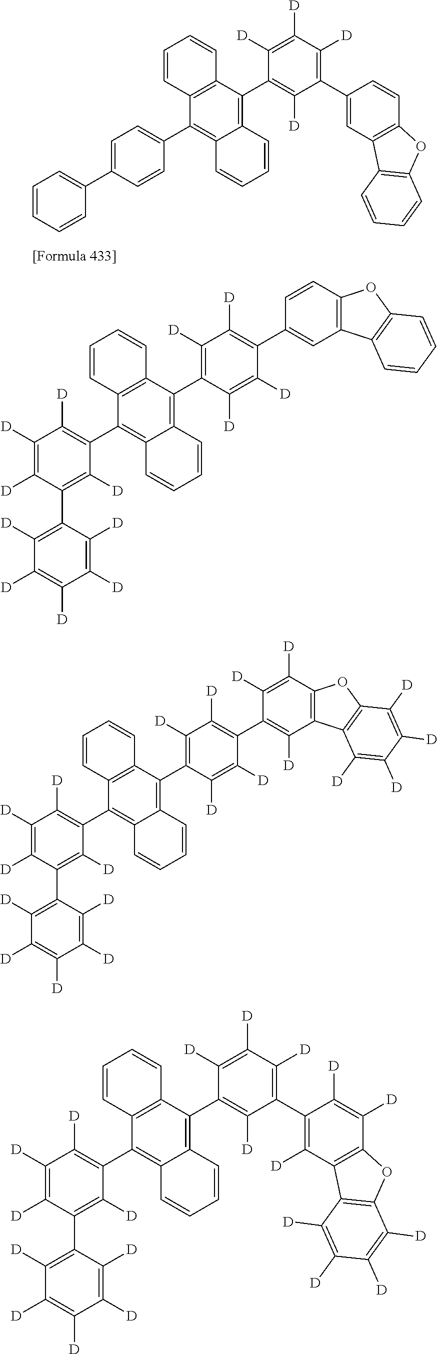

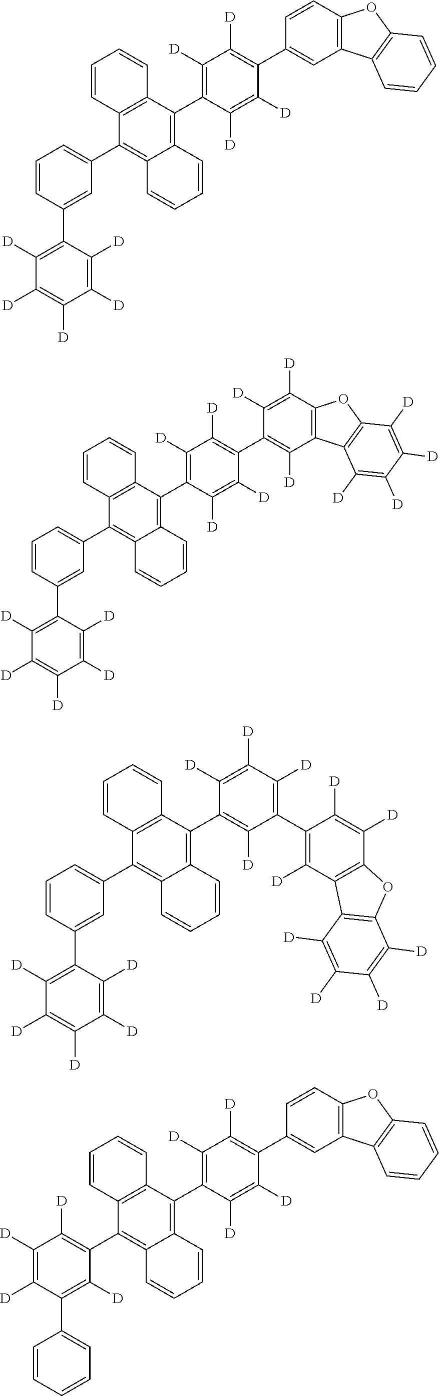

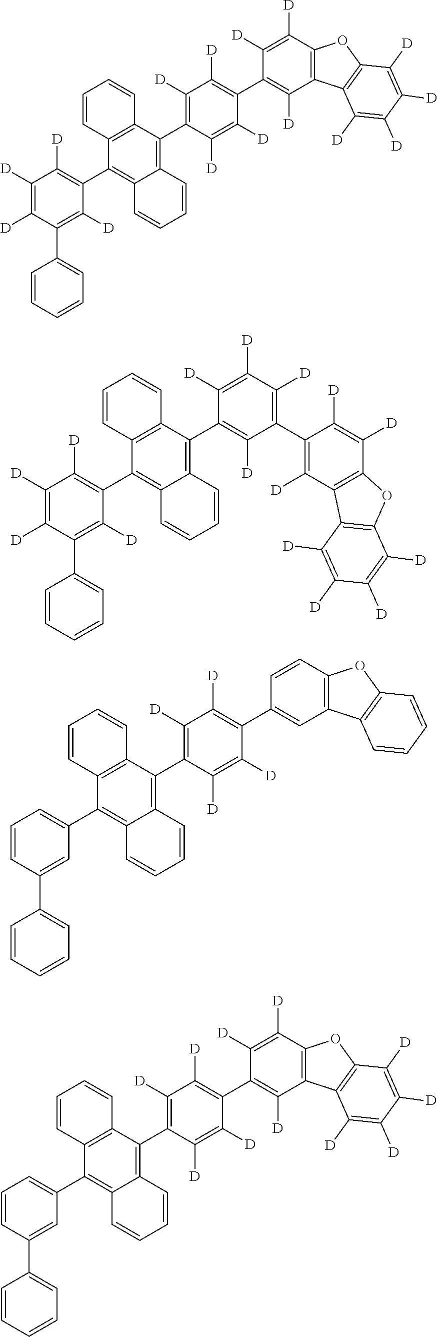

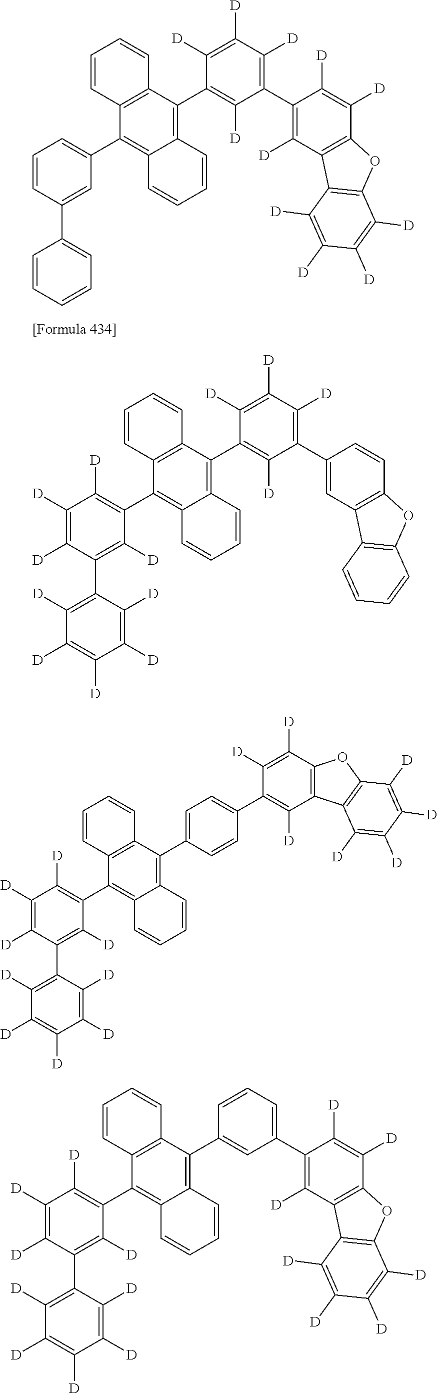

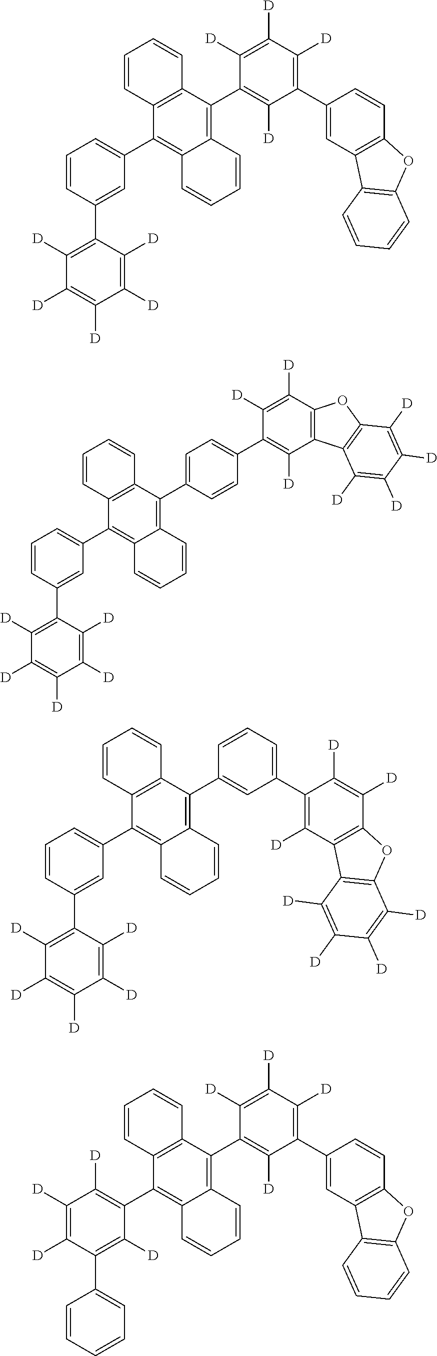

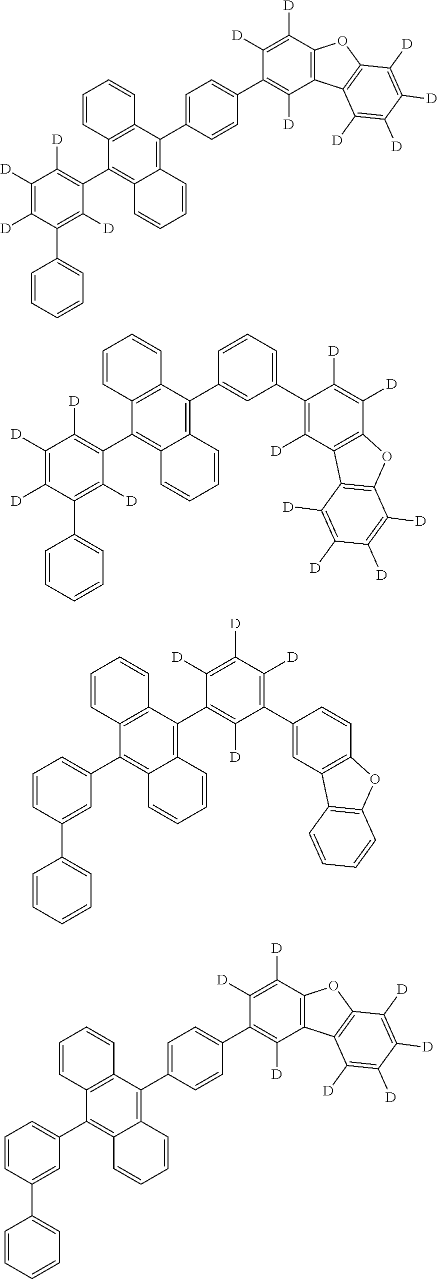 [SMILES: [2H]c1c([2H])c(-c2ccccc2)c([2H])c(-c2c3ccccc3c(-c3ccc(-c4c([2H])c([2H])c5oc6c([2H])c([2H])c([2H])c([2H])c6c5c4[2H])cc3)c3ccccc23)c1[2H] ; [2H]c1c([2H])c(-c2ccccc2)c([2H])c(-c2c3ccccc3c(-c3cccc(-c4c([2H])c([2H])c5oc6c([2H])c([2H])c([2H])c([2H])c6c5c4[2H])c3)c3ccccc23)c1[2H] ; [2H]c1c([2H])c(-c2ccc3oc4ccccc4c3c2)c([2H])c(-c2c3ccccc3c(-c3cccc(-c4ccccc4)c3)c3ccccc23)c1[2H] ; [2H]c1c([2H])c([2H])c2c(oc3c([2H])c([2H])c(-c4ccc(-c5c6ccccc6c(-c6cccc(-c7ccccc7)c6)c6ccccc56)cc4)c([2H])c32)c1[2H]]

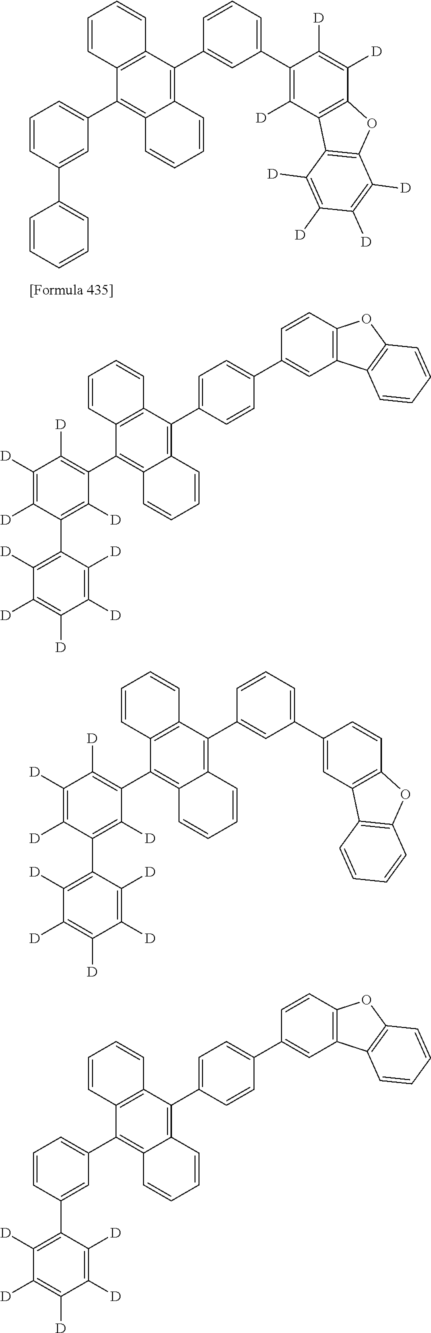 [SMILES: [2H]c1c([2H])c([2H])c2c(oc3c([2H])c([2H])c(-c4cccc(-c5c6ccccc6c(-c6cccc(-c7ccccc7)c6)c6ccccc56)c4)c([2H])c32)c1[2H] ; [2H]c1c([2H])c([2H])c(-c2c([2H])c([2H])c([2H])c(-c3c4ccccc4c(-c4ccc(-c5ccc6oc7ccccc7c6c5)cc4)c4ccccc34)c2[2H])c([2H])c1[2H] ; [2H]c1c([2H])c([2H])c(-c2c([2H])c([2H])c([2H])c(-c3c4ccccc4c(-c4cccc(-c5ccc6oc7ccccc7c6c5)c4)c4ccccc34)c2[2H])c([2H])c1[2H] ; [2H]c1c([2H])c([2H])c(-c2cccc(-c3c4ccccc4c(-c4ccc(-c5ccc6oc7ccccc7c6c5)cc4)c4ccccc34)c2)c([2H])c1[2H]]

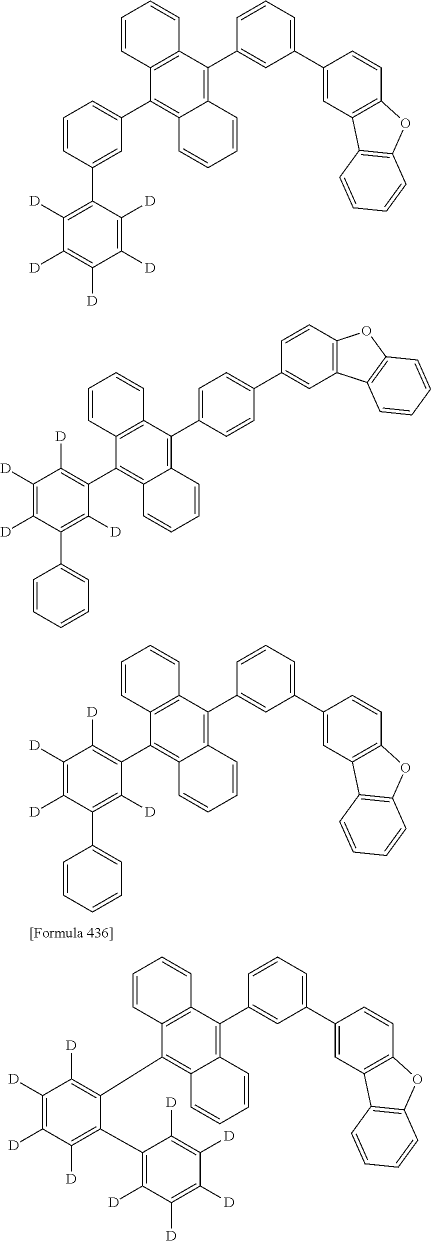 [SMILES: [2H]c1c([2H])c([2H])c(-c2cccc(-c3c4ccccc4c(-c4cccc(-c5ccc6oc7ccccc7c6c5)c4)c4ccccc34)c2)c([2H])c1[2H] ; [2H]c1c([2H])c(-c2ccccc2)c([2H])c(-c2c3ccccc3c(-c3ccc(-c4ccc5oc6ccccc6c5c4)cc3)c3ccccc23)c1[2H] ; [2H]c1c([2H])c(-c2ccccc2)c([2H])c(-c2c3ccccc3c(-c3cccc(-c4ccc5oc6ccccc6c5c4)c3)c3ccccc23)c1[2H] ; [2H]c1c([2H])c([2H])c(-c2c([2H])c([2H])c([2H])c([2H])c2-c2c3ccccc3c(-c3cccc(-c4ccc5oc6ccccc6c5c4)c3)c3ccccc23)c([2H])c1[2H]]

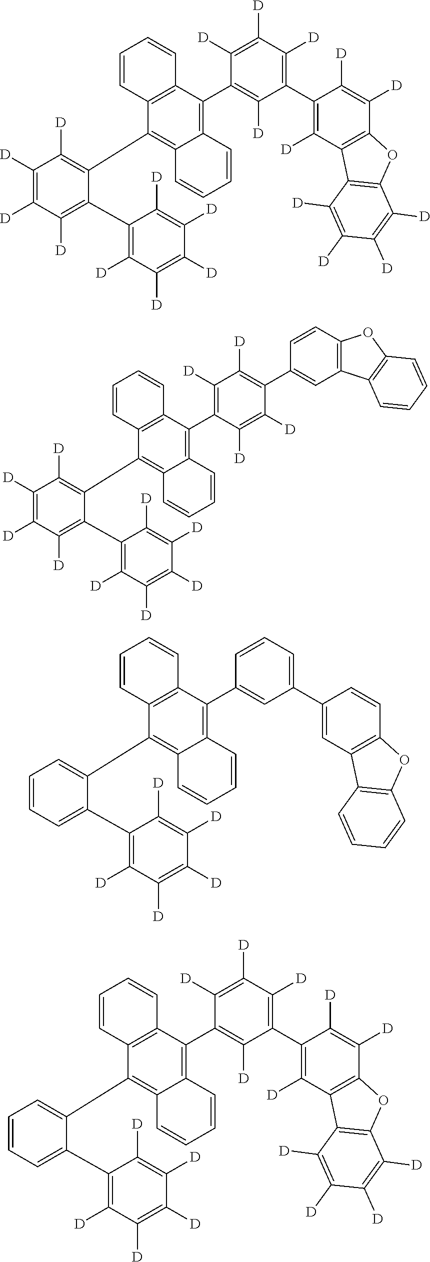

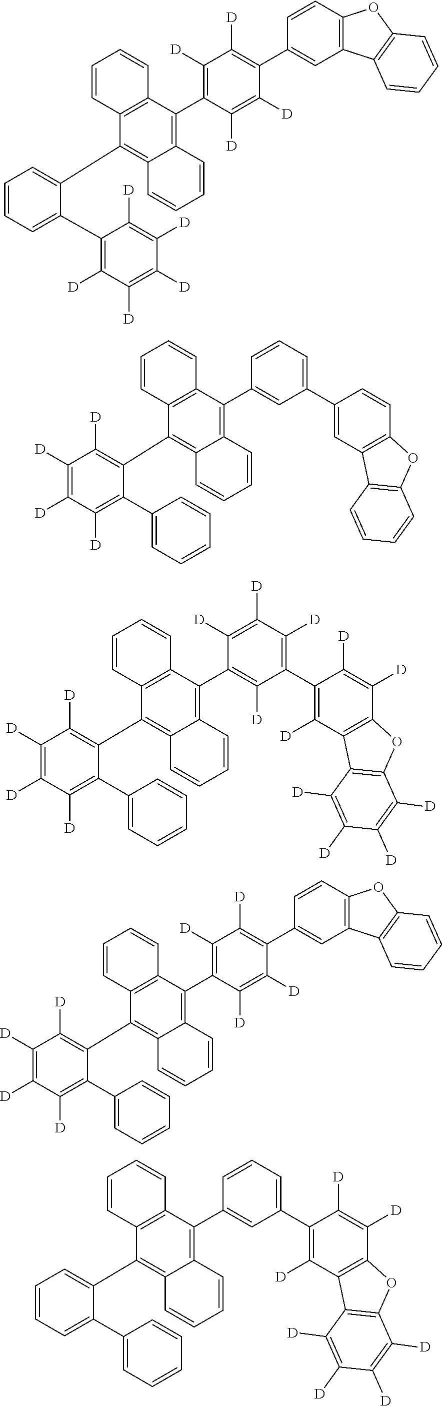

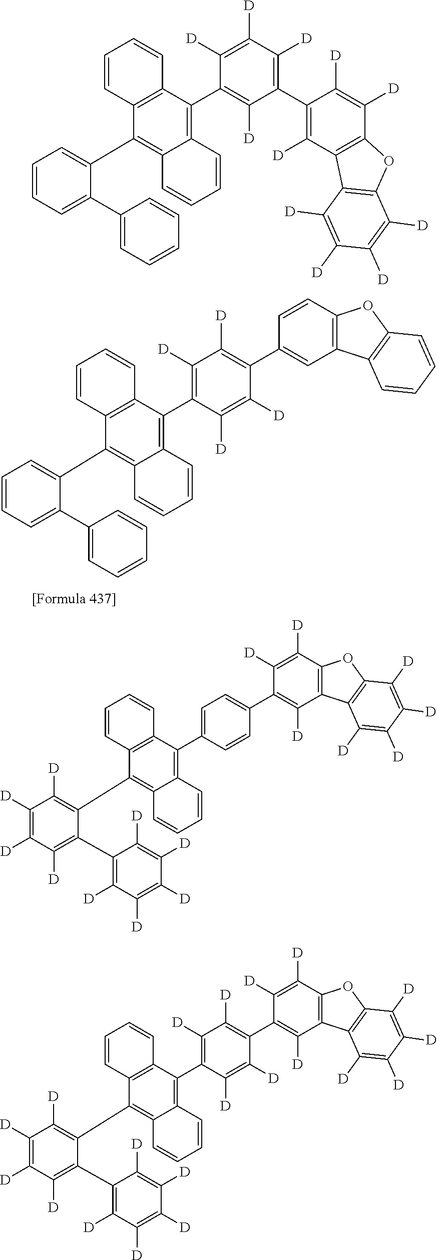

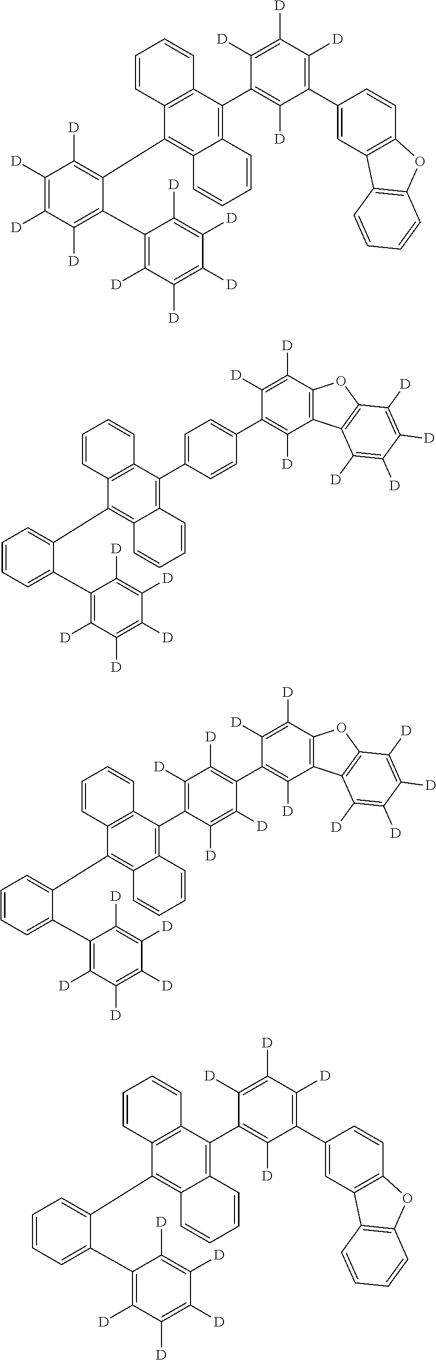

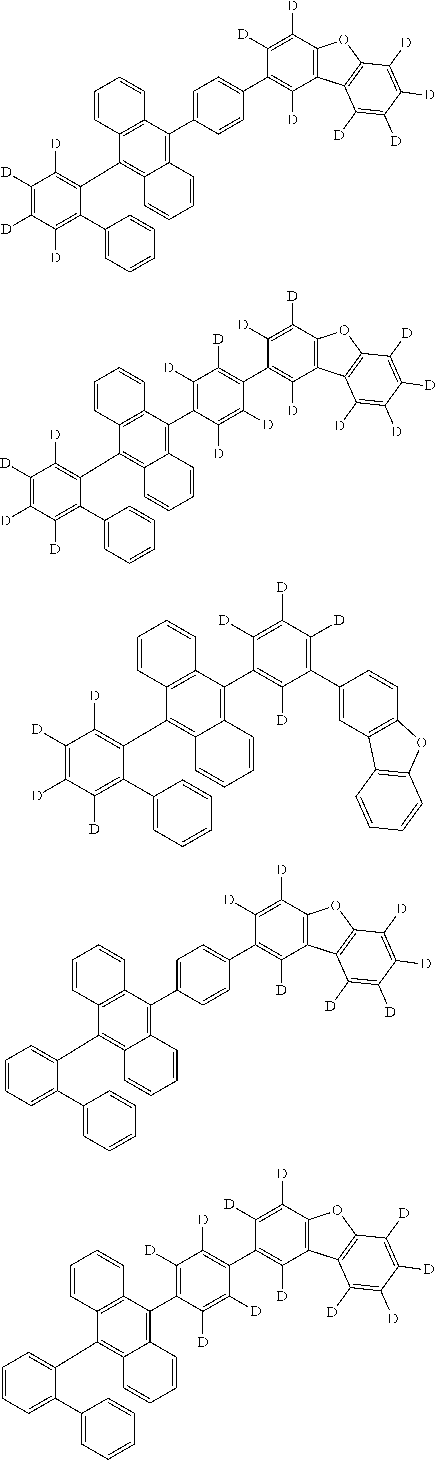

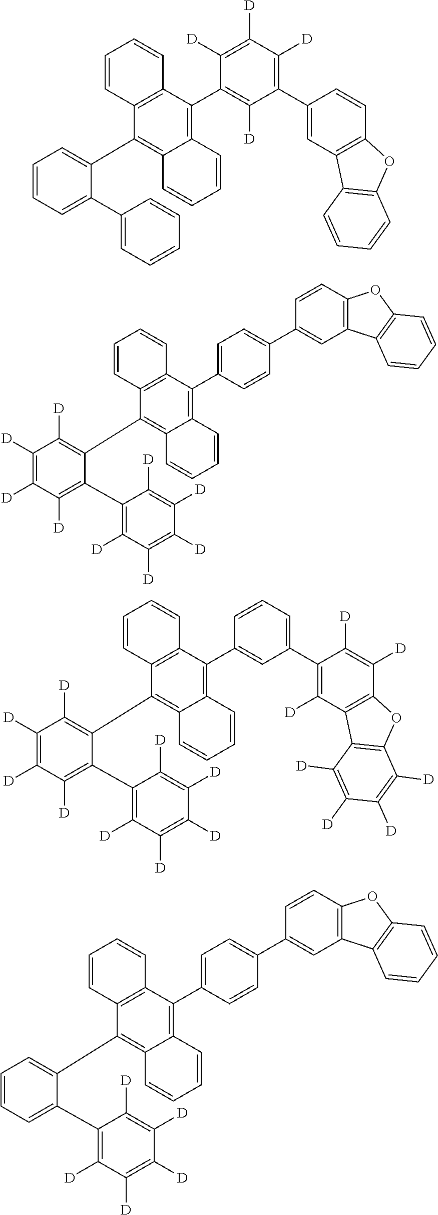

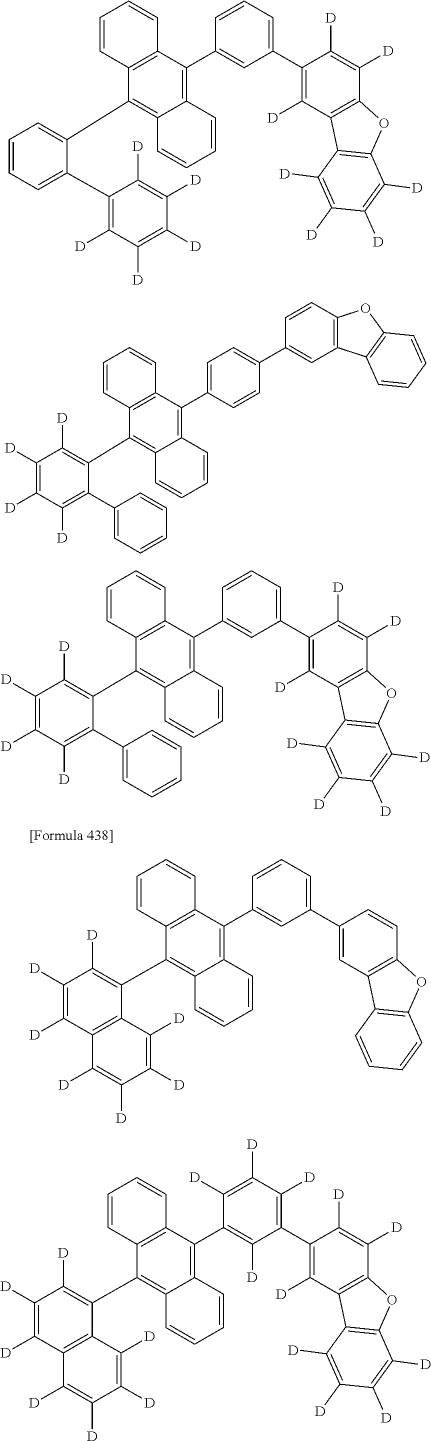

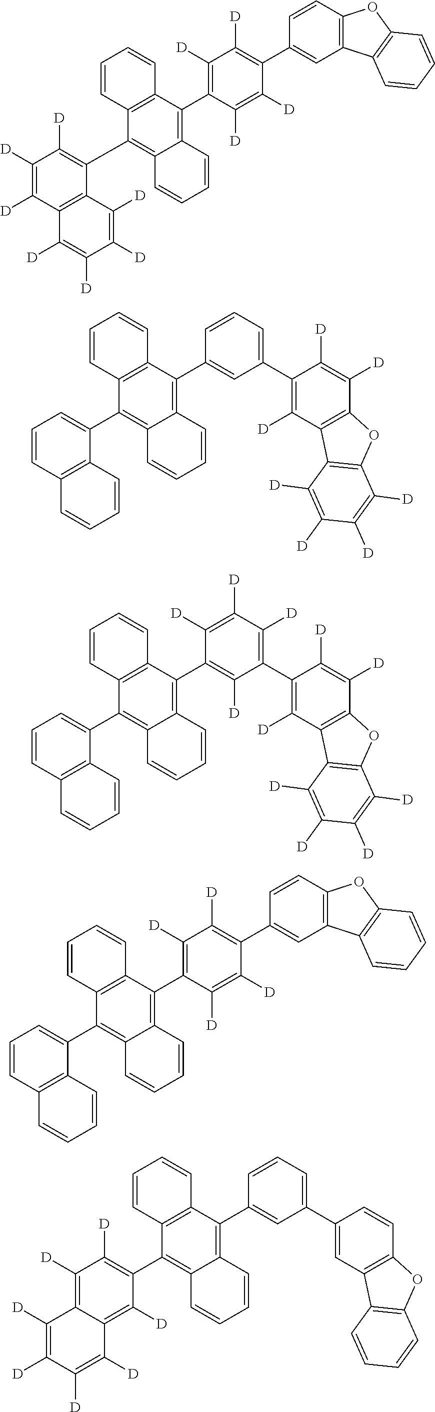

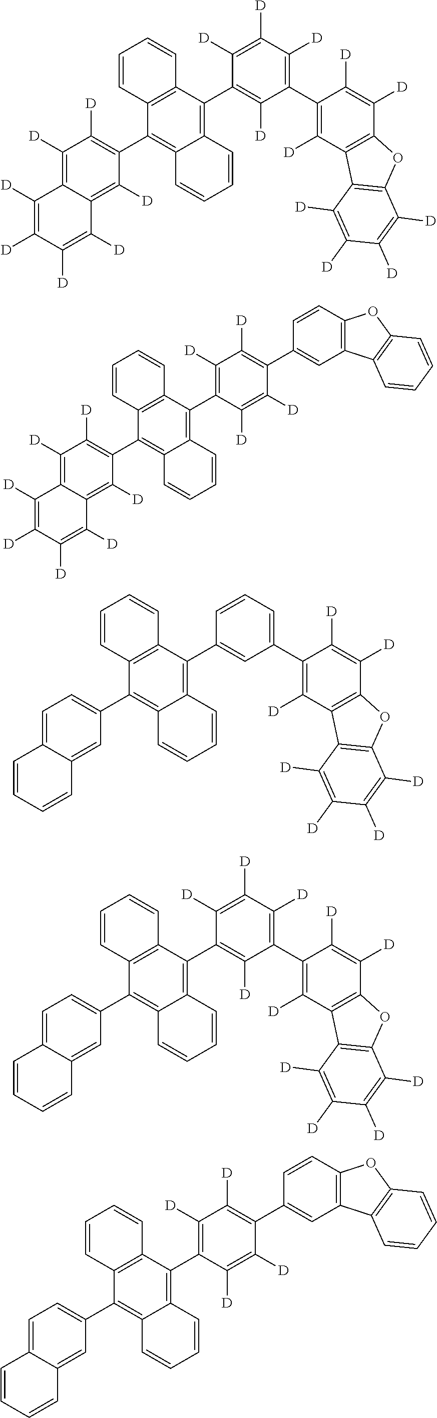

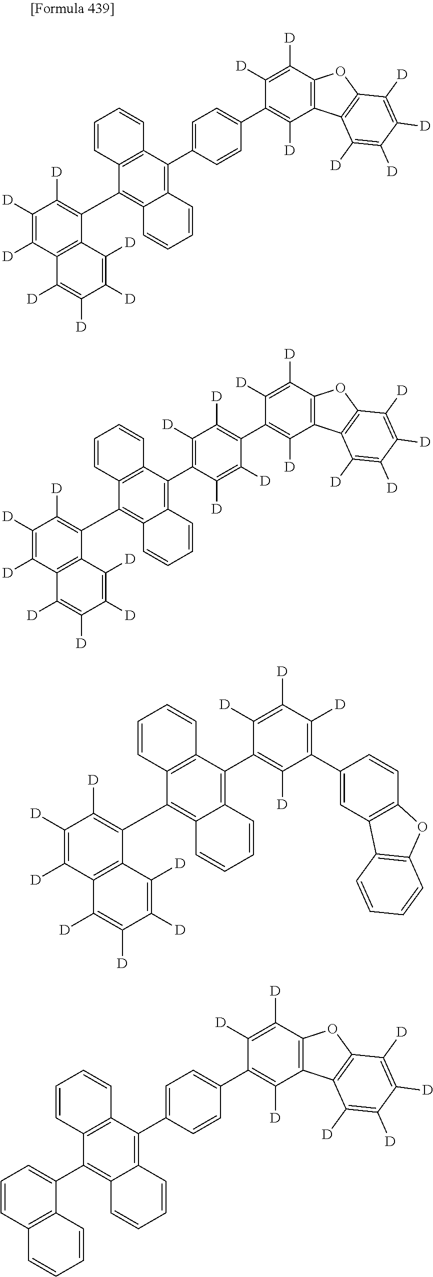

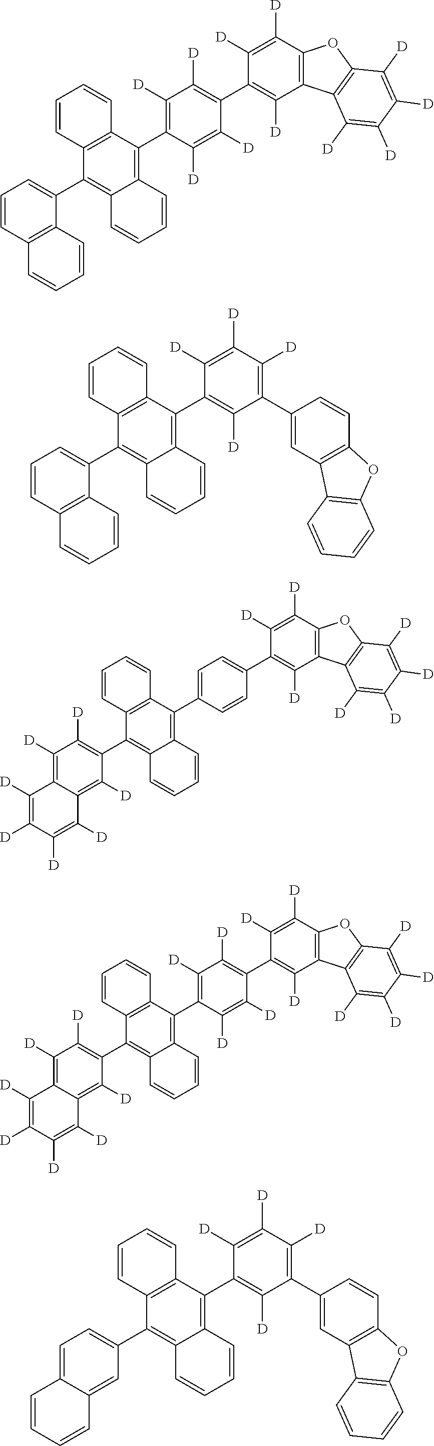

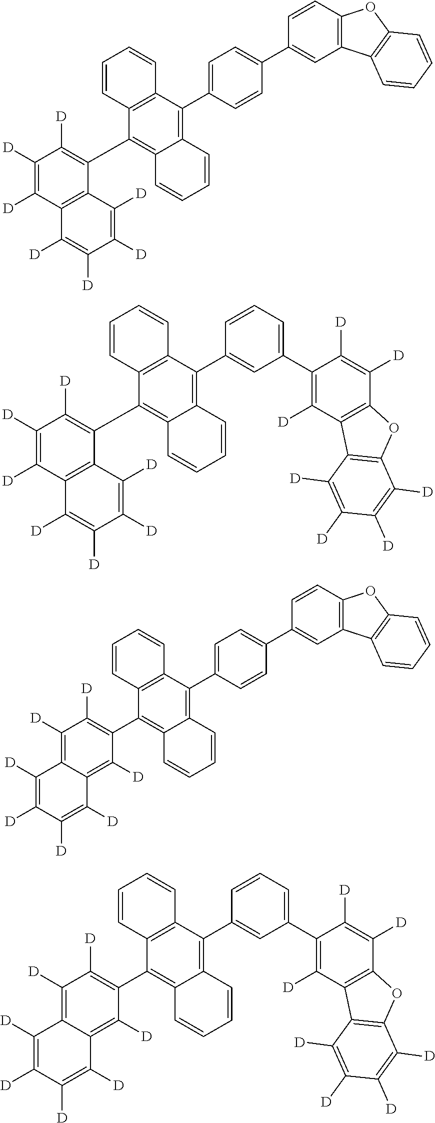

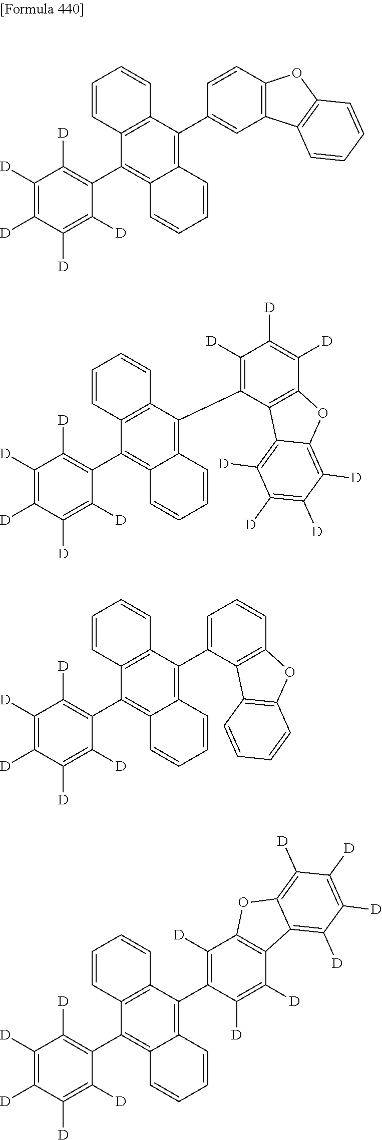 [SMILES: [2H]c1c([2H])c([2H])c(-c2c3ccccc3c(-c3ccc4oc5ccccc5c4c3)c3ccccc23)c([2H])c1[2H] ; [2H]c1c([2H])c([2H])c(-c2c3ccccc3c(-c3c([2H])c([2H])c([2H])c4oc5c([2H])c([2H])c([2H])c([2H])c5c34)c3ccccc23)c([2H])c1[2H] ; [2H]c1c([2H])c([2H])c(-c2c3ccccc3c(-c3cccc4oc5ccccc5c34)c3ccccc23)c([2H])c1[2H] ; [2H]c1c([2H])c([2H])c(-c2c3ccccc3c(-c3c([2H])c([2H])c4c(oc5c([2H])c([2H])c([2H])c([2H])c54)c3[2H])c3ccccc23)c([2H])c1[2H]]

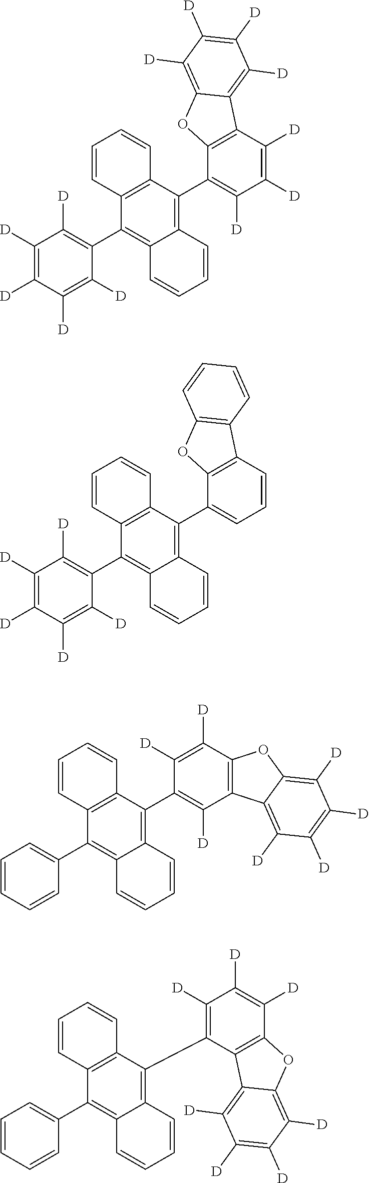 [SMILES: [2H]c1c([2H])c([2H])c(-c2c3ccccc3c(-c3c([2H])c([2H])c([2H])c4c3oc3c([2H])c([2H])c([2H])c([2H])c34)c3ccccc23)c([2H])c1[2H] ; [2H]c1c([2H])c([2H])c(-c2c3ccccc3c(-c3cccc4c3oc3ccccc34)c3ccccc23)c([2H])c1[2H] ; [2H]c1c([2H])c([2H])c2c(oc3c([2H])c([2H])c(-c4c5ccccc5c(-c5ccccc5)c5ccccc45)c([2H])c32)c1[2H] ; [2H]c1c([2H])c([2H])c2c(oc3c([2H])c([2H])c([2H])c(-c4c5ccccc5c(-c5ccccc5)c5ccccc45)c32)c1[2H]]

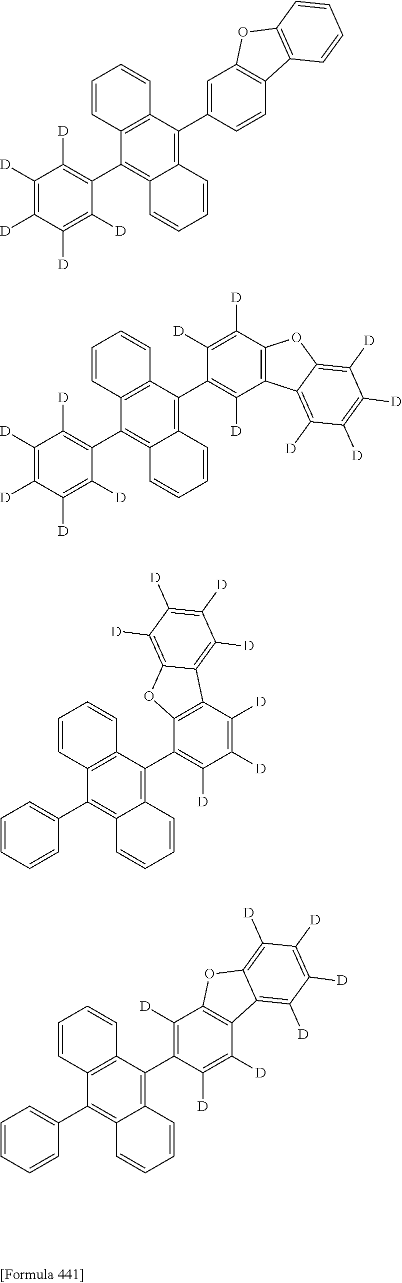 [SMILES: [2H]c1c([2H])c([2H])c(-c2c3ccccc3c(-c3ccc4c(c3)oc3ccccc34)c3ccccc23)c([2H])c1[2H] ; [2H]c1c([2H])c([2H])c(-c2c3ccccc3c(-c3c([2H])c([2H])c4oc5c([2H])c([2H])c([2H])c([2H])c5c4c3[2H])c3ccccc23)c([2H])c1[2H] ; [2H]c1c([2H])c([2H])c2c(oc3c(-c4c5ccccc5c(-c5ccccc5)c5ccccc45)c([2H])c([2H])c([2H])c32)c1[2H] ; [2H]c1c([2H])c([2H])c2c(oc3c([2H])c(-c4c5ccccc5c(-c5ccccc5)c5ccccc45)c([2H])c([2H])c32)c1[2H]]

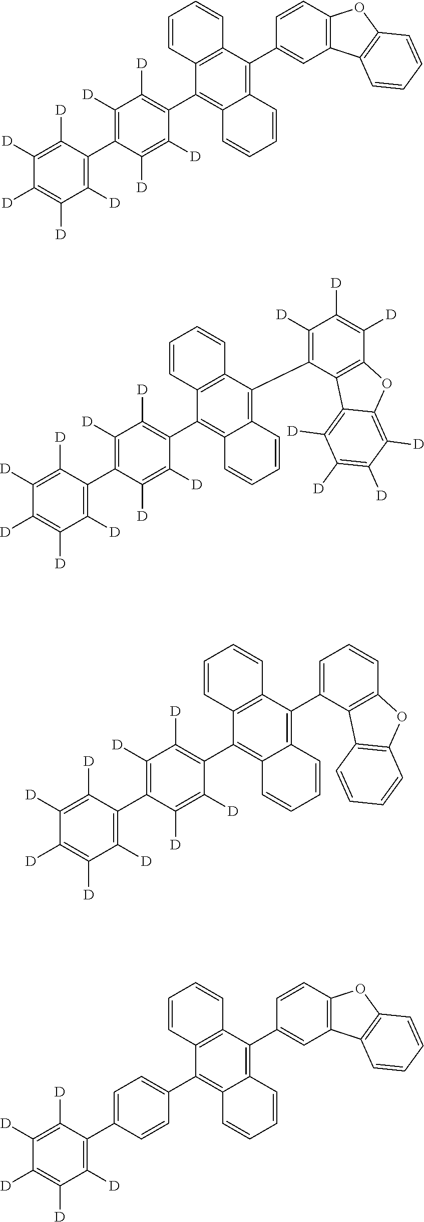 [SMILES: [2H]c1c([2H])c([2H])c(-c2c([2H])c([2H])c(-c3c4ccccc4c(-c4ccc5oc6ccccc6c5c4)c4ccccc34)c([2H])c2[2H])c([2H])c1[2H] ; [2H]c1c([2H])c([2H])c(-c2c([2H])c([2H])c(-c3c4ccccc4c(-c4c([2H])c([2H])c([2H])c5oc6c([2H])c([2H])c([2H])c([2H])c6c45)c4ccccc34)c([2H])c2[2H])c([2H])c1[2H] ; [2H]c1c([2H])c([2H])c(-c2c([2H])c([2H])c(-c3c4ccccc4c(-c4cccc5oc6ccccc6c45)c4ccccc34)c([2H])c2[2H])c([2H])c1[2H] ; [2H]c1c([2H])c([2H])c(-c2ccc(-c3c4ccccc4c(-c4ccc5oc6ccccc6c5c4)c4ccccc34)cc2)c([2H])c1[2H]]

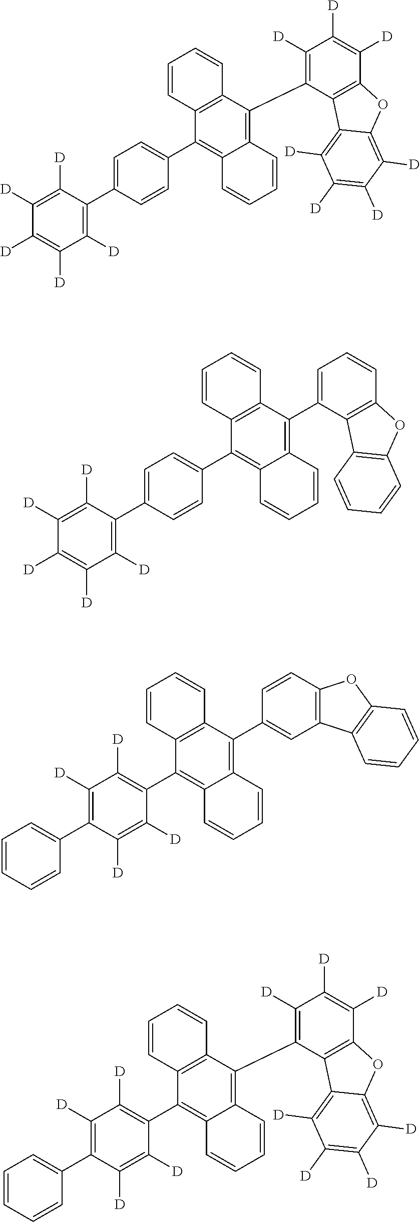 [SMILES: [2H]c1c([2H])c([2H])c(-c2ccc(-c3c4ccccc4c(-c4c([2H])c([2H])c([2H])c5oc6c([2H])c([2H])c([2H])c([2H])c6c45)c4ccccc34)cc2)c([2H])c1[2H] ; [2H]c1c([2H])c([2H])c(-c2ccc(-c3c4ccccc4c(-c4cccc5oc6ccccc6c45)c4ccccc34)cc2)c([2H])c1[2H] ; [2H]c1c([2H])c(-c2c3ccccc3c(-c3ccc4oc5ccccc5c4c3)c3ccccc23)c([2H])c([2H])c1-c1ccccc1 ; [2H]c1c([2H])c(-c2c3ccccc3c(-c3c([2H])c([2H])c([2H])c4oc5c([2H])c([2H])c([2H])c([2H])c5c34)c3ccccc23)c([2H])c([2H])c1-c1ccccc1]

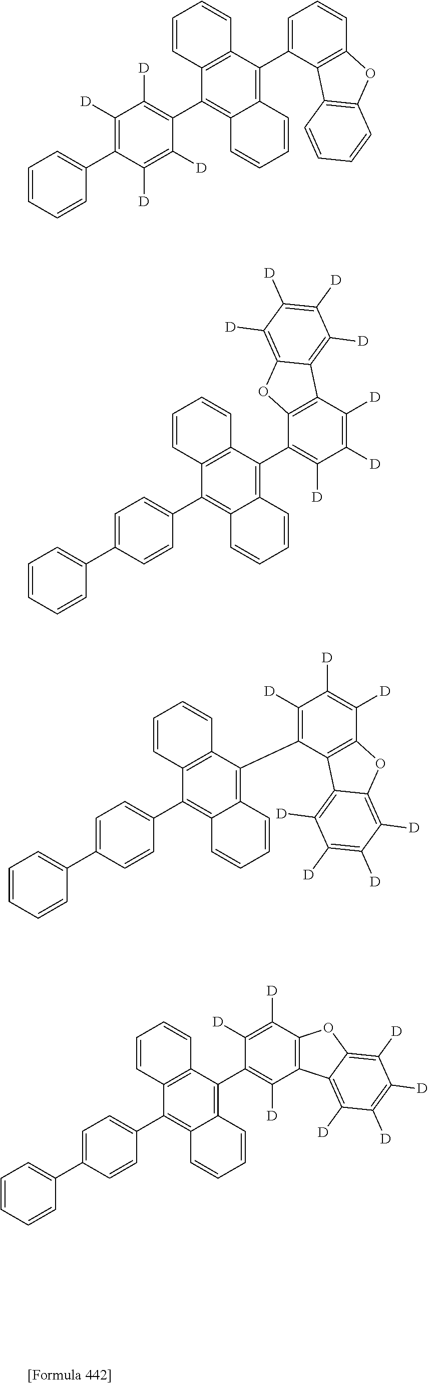 [SMILES: [2H]c1c([2H])c(-c2c3ccccc3c(-c3cccc4oc5ccccc5c34)c3ccccc23)c([2H])c([2H])c1-c1ccccc1 ; [2H]c1c([2H])c([2H])c2c(oc3c(-c4c5ccccc5c(-c5ccc(-c6ccccc6)cc5)c5ccccc45)c([2H])c([2H])c([2H])c32)c1[2H] ; [2H]c1c([2H])c([2H])c2c(oc3c([2H])c([2H])c([2H])c(-c4c5ccccc5c(-c5ccc(-c6ccccc6)cc5)c5ccccc45)c32)c1[2H] ; [2H]c1c([2H])c([2H])c2c(oc3c([2H])c([2H])c(-c4c5ccccc5c(-c5ccc(-c6ccccc6)cc5)c5ccccc45)c([2H])c32)c1[2H]]

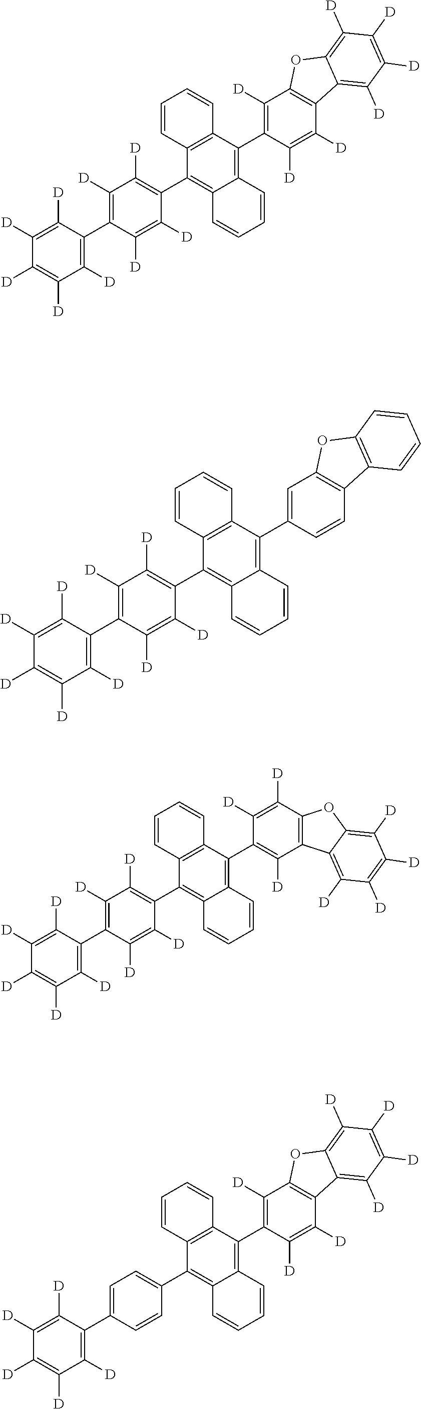

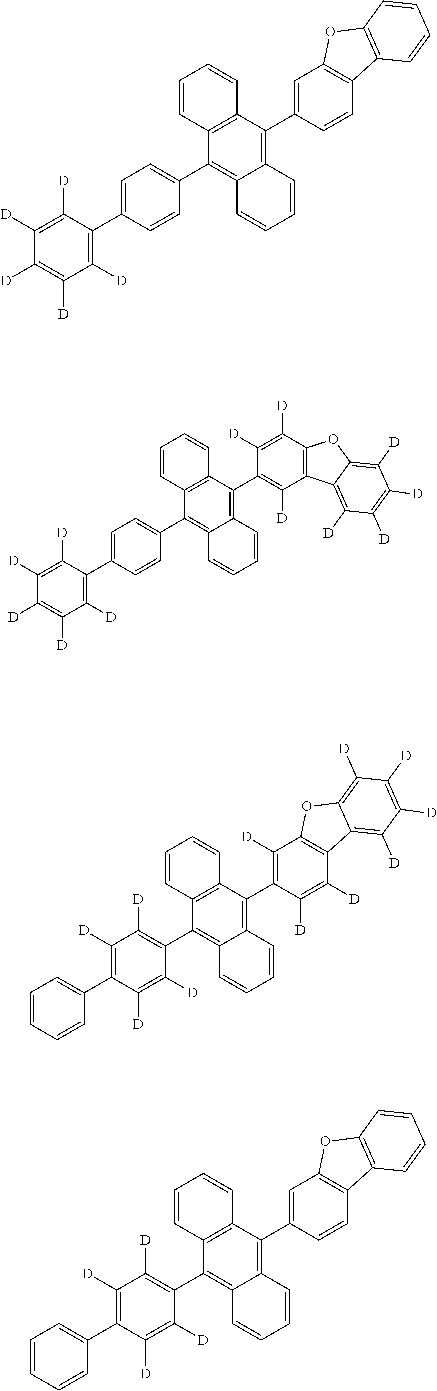 [SMILES: [2H]c1c([2H])c([2H])c(-c2ccc(-c3c4ccccc4c(-c4ccc5c(c4)oc4ccccc45)c4ccccc34)cc2)c([2H])c1[2H] ; [2H]c1c([2H])c([2H])c(-c2ccc(-c3c4ccccc4c(-c4c([2H])c([2H])c5oc6c([2H])c([2H])c([2H])c([2H])c6c5c4[2H])c4ccccc34)cc2)c([2H])c1[2H] ; [2H]c1c([2H])c(-c2c3ccccc3c(-c3c([2H])c([2H])c4c(oc5c([2H])c([2H])c([2H])c([2H])c54)c3[2H])c3ccccc23)c([2H])c([2H])c1-c1ccccc1 ; [2H]c1c([2H])c(-c2c3ccccc3c(-c3ccc4c(c3)oc3ccccc34)c3ccccc23)c([2H])c([2H])c1-c1ccccc1]

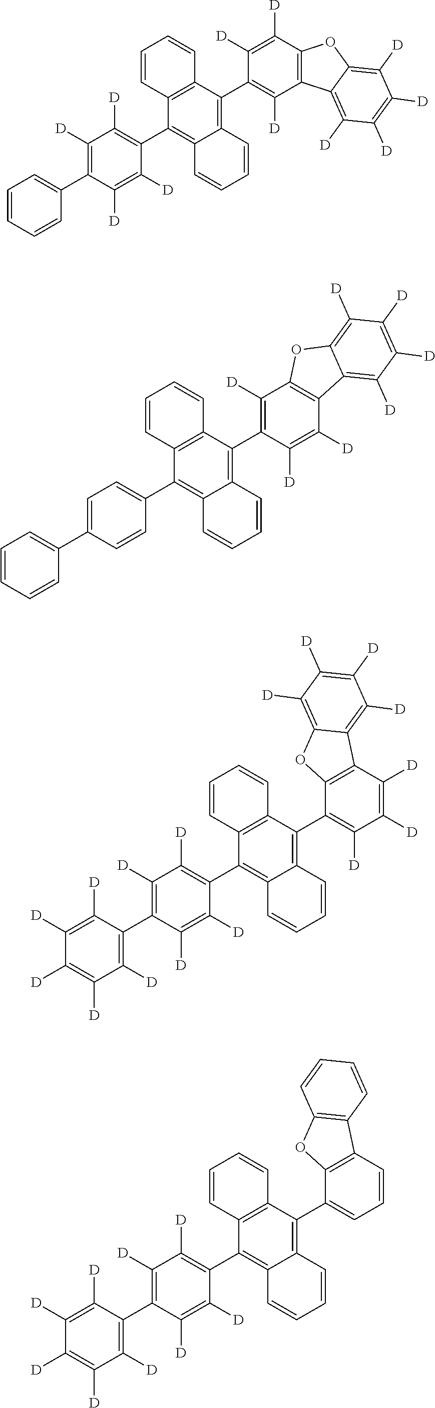

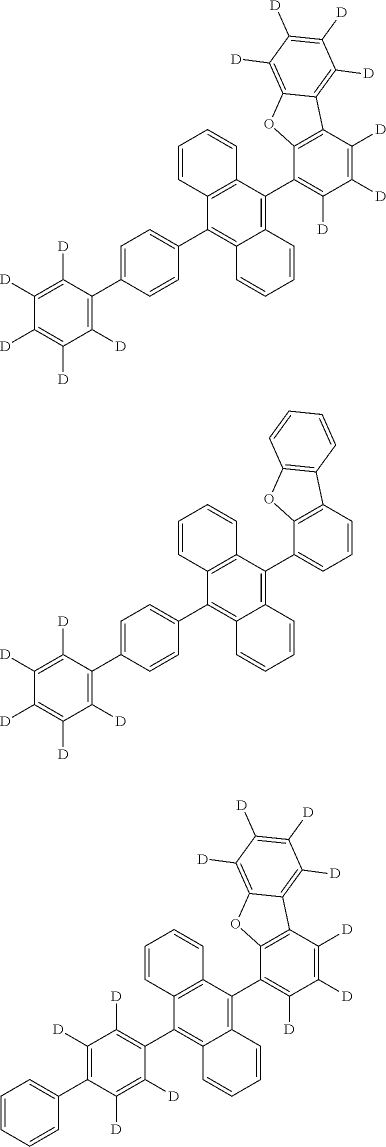 [SMILES: [2H]c1c([2H])c([2H])c(-c2ccc(-c3c4ccccc4c(-c4c([2H])c([2H])c([2H])c5c4oc4c([2H])c([2H])c([2H])c([2H])c45)c4ccccc34)cc2)c([2H])c1[2H] ; [2H]c1c([2H])c([2H])c(-c2ccc(-c3c4ccccc4c(-c4cccc5c4oc4ccccc45)c4ccccc34)cc2)c([2H])c1[2H] ; [2H]c1c([2H])c(-c2c3ccccc3c(-c3c([2H])c([2H])c([2H])c4c3oc3c([2H])c([2H])c([2H])c([2H])c34)c3ccccc23)c([2H])c([2H])c1-c1ccccc1]

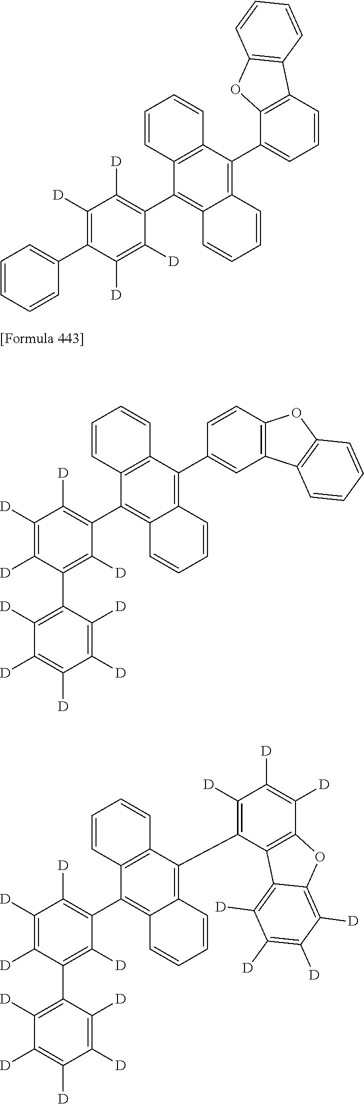 [SMILES: [2H]c1c([2H])c(-c2c3ccccc3c(-c3cccc4c3oc3ccccc34)c3ccccc23)c([2H])c([2H])c1-c1ccccc1 ; [2H]c1c([2H])c([2H])c(-c2c([2H])c([2H])c([2H])c(-c3c4ccccc4c(-c4ccc5oc6ccccc6c5c4)c4ccccc34)c2[2H])c([2H])c1[2H] ; [2H]c1c([2H])c([2H])c(-c2c([2H])c([2H])c([2H])c(-c3c4ccccc4c(-c4c([2H])c([2H])c([2H])c5oc6c([2H])c([2H])c([2H])c([2H])c6c45)c4ccccc34)c2[2H])c([2H])c1[2H]]

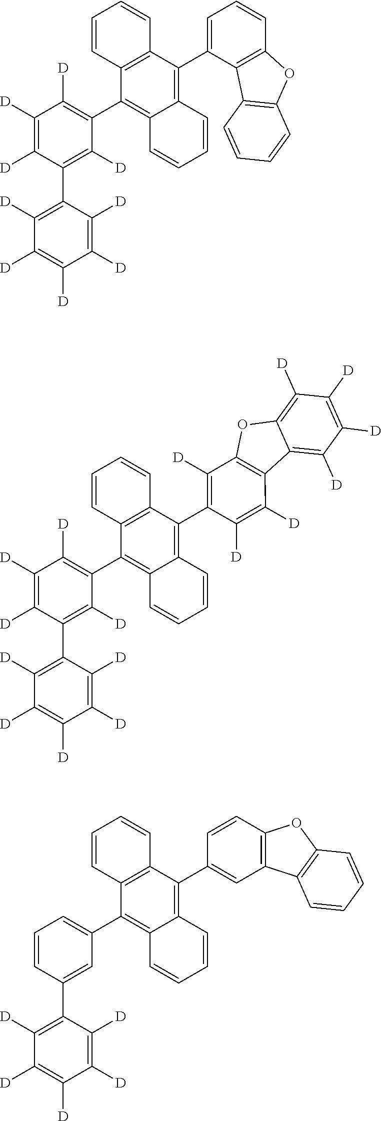 [SMILES: [2H]c1c([2H])c([2H])c(-c2c([2H])c([2H])c([2H])c(-c3c4ccccc4c(-c4cccc5oc6ccccc6c45)c4ccccc34)c2[2H])c([2H])c1[2H] ; [2H]c1c([2H])c([2H])c(-c2c([2H])c([2H])c([2H])c(-c3c4ccccc4c(-c4c([2H])c([2H])c5c(oc6c([2H])c([2H])c([2H])c([2H])c65)c4[2H])c4ccccc34)c2[2H])c([2H])c1[2H] ; [2H]c1c([2H])c([2H])c(-c2cccc(-c3c4ccccc4c(-c4ccc5oc6ccccc6c5c4)c4ccccc34)c2)c([2H])c1[2H]]

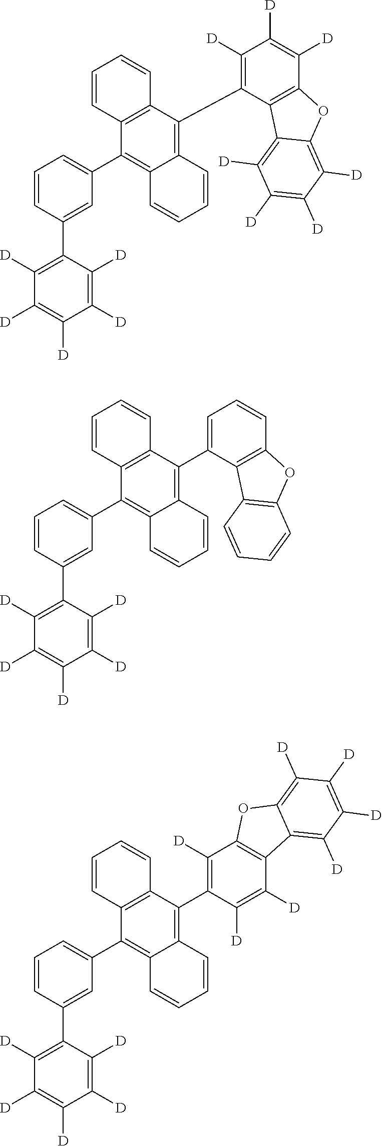 [SMILES: [2H]c1c([2H])c([2H])c(-c2cccc(-c3c4ccccc4c(-c4c([2H])c([2H])c([2H])c5oc6c([2H])c([2H])c([2H])c([2H])c6c45)c4ccccc34)c2)c([2H])c1[2H] ; [2H]c1c([2H])c([2H])c(-c2cccc(-c3c4ccccc4c(-c4cccc5oc6ccccc6c45)c4ccccc34)c2)c([2H])c1[2H] ; [2H]c1c([2H])c([2H])c(-c2cccc(-c3c4ccccc4c(-c4c([2H])c([2H])c5c(oc6c([2H])c([2H])c([2H])c([2H])c65)c4[2H])c4ccccc34)c2)c([2H])c1[2H]]

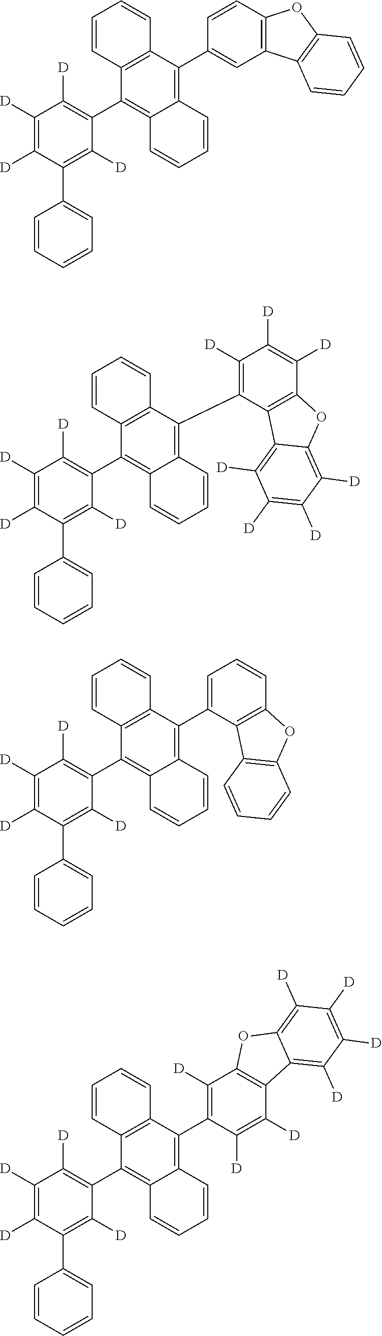 [SMILES: [2H]c1c([2H])c(-c2ccccc2)c([2H])c(-c2c3ccccc3c(-c3ccc4oc5ccccc5c4c3)c3ccccc23)c1[2H] ; [2H]c1c([2H])c(-c2ccccc2)c([2H])c(-c2c3ccccc3c(-c3c([2H])c([2H])c([2H])c4oc5c([2H])c([2H])c([2H])c([2H])c5c34)c3ccccc23)c1[2H] ; [2H]c1c([2H])c(-c2ccccc2)c([2H])c(-c2c3ccccc3c(-c3cccc4oc5ccccc5c34)c3ccccc23)c1[2H] ; [2H]c1c([2H])c(-c2ccccc2)c([2H])c(-c2c3ccccc3c(-c3c([2H])c([2H])c4c(oc5c([2H])c([2H])c([2H])c([2H])c54)c3[2H])c3ccccc23)c1[2H]]

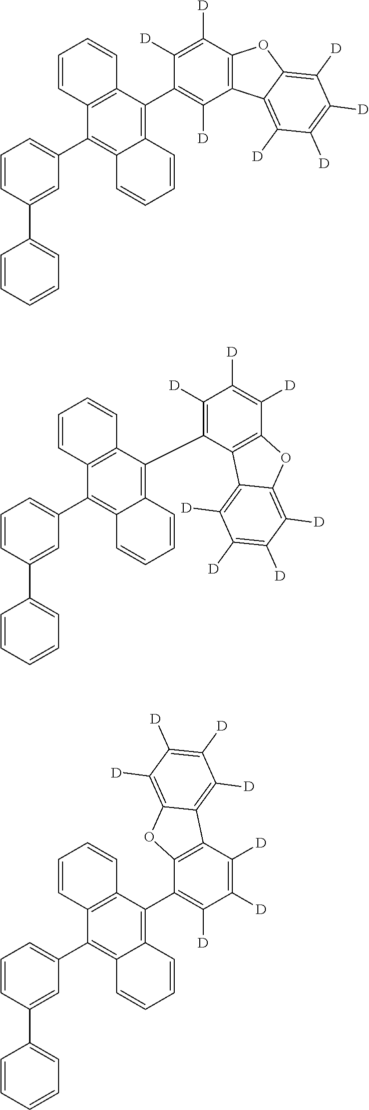 [SMILES: [2H]c1c([2H])c([2H])c2c(oc3c([2H])c([2H])c(-c4c5ccccc5c(-c5cccc(-c6ccccc6)c5)c5ccccc45)c([2H])c32)c1[2H] ; [2H]c1c([2H])c([2H])c2c(oc3c([2H])c([2H])c([2H])c(-c4c5ccccc5c(-c5cccc(-c6ccccc6)c5)c5ccccc45)c32)c1[2H] ; [2H]c1c([2H])c([2H])c2c(oc3c(-c4c5ccccc5c(-c5cccc(-c6ccccc6)c5)c5ccccc45)c([2H])c([2H])c([2H])c32)c1[2H]]

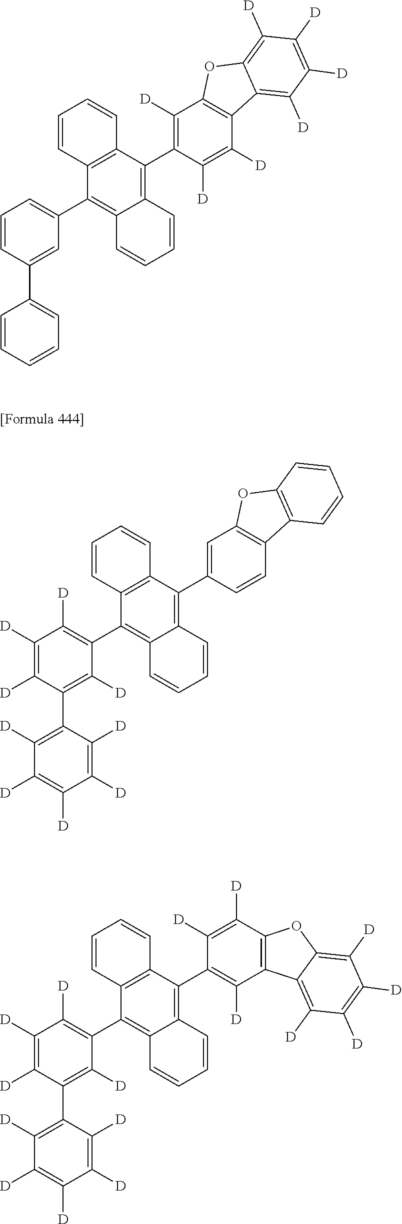 [SMILES: [2H]c1c([2H])c([2H])c2c(oc3c([2H])c(-c4c5ccccc5c(-c5cccc(-c6ccccc6)c5)c5ccccc45)c([2H])c([2H])c32)c1[2H] ; [2H]c1c([2H])c([2H])c(-c2c([2H])c([2H])c([2H])c(-c3c4ccccc4c(-c4ccc5c(c4)oc4ccccc45)c4ccccc34)c2[2H])c([2H])c1[2H] ; [2H]c1c([2H])c([2H])c(-c2c([2H])c([2H])c([2H])c(-c3c4ccccc4c(-c4c([2H])c([2H])c5oc6c([2H])c([2H])c([2H])c([2H])c6c5c4[2H])c4ccccc34)c2[2H])c([2H])c1[2H]]

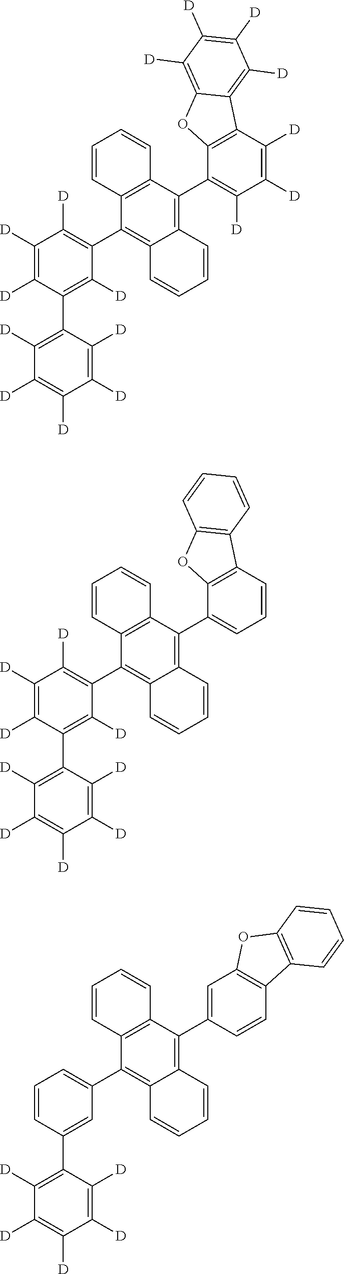 [SMILES: [2H]c1c([2H])c([2H])c(-c2c([2H])c([2H])c([2H])c(-c3c4ccccc4c(-c4c([2H])c([2H])c([2H])c5c4oc4c([2H])c([2H])c([2H])c([2H])c45)c4ccccc34)c2[2H])c([2H])c1[2H] ; [2H]c1c([2H])c([2H])c(-c2c([2H])c([2H])c([2H])c(-c3c4ccccc4c(-c4cccc5c4oc4ccccc45)c4ccccc34)c2[2H])c([2H])c1[2H] ; [2H]c1c([2H])c([2H])c(-c2cccc(-c3c4ccccc4c(-c4ccc5c(c4)oc4ccccc45)c4ccccc34)c2)c([2H])c1[2H]]

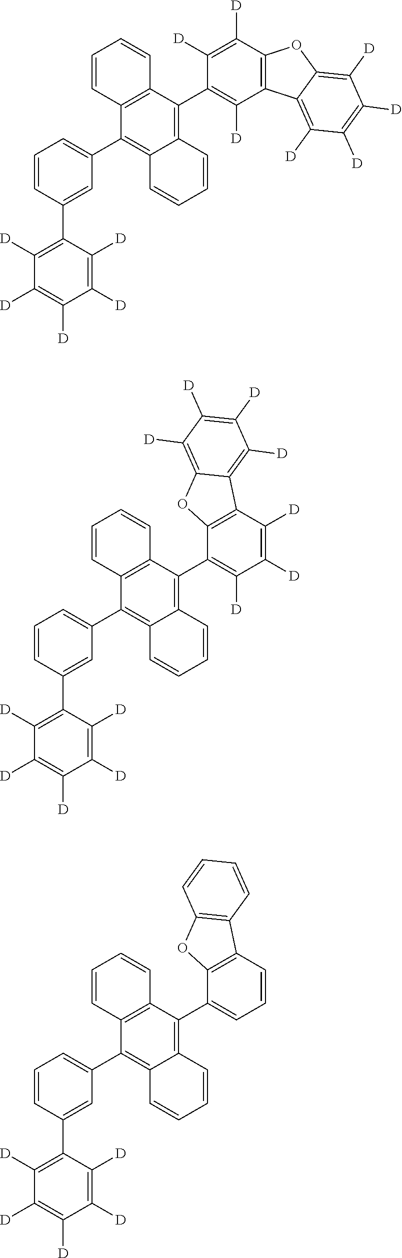 [SMILES: [2H]c1c([2H])c([2H])c(-c2cccc(-c3c4ccccc4c(-c4c([2H])c([2H])c5oc6c([2H])c([2H])c([2H])c([2H])c6c5c4[2H])c4ccccc34)c2)c([2H])c1[2H] ; [2H]c1c([2H])c([2H])c(-c2cccc(-c3c4ccccc4c(-c4c([2H])c([2H])c([2H])c5c4oc4c([2H])c([2H])c([2H])c([2H])c45)c4ccccc34)c2)c([2H])c1[2H] ; [2H]c1c([2H])c([2H])c(-c2cccc(-c3c4ccccc4c(-c4cccc5c4oc4ccccc45)c4ccccc34)c2)c([2H])c1[2H]]

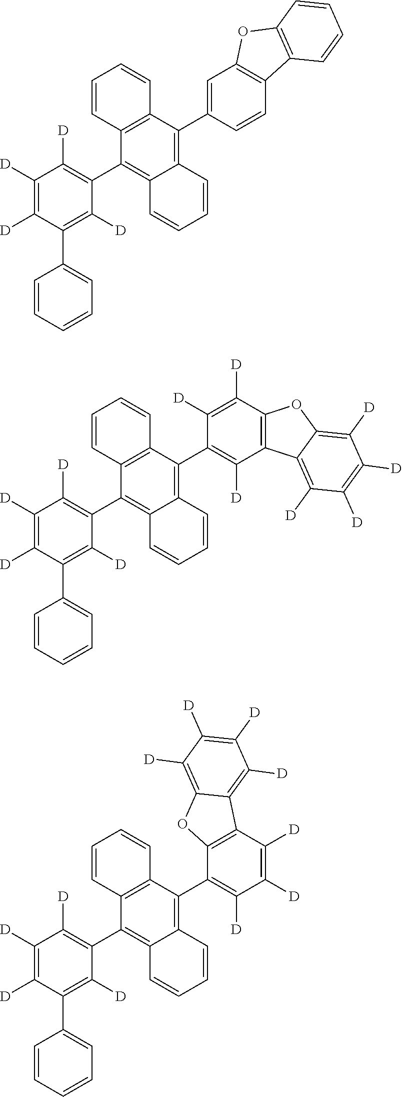 [SMILES: [2H]c1c([2H])c(-c2ccccc2)c([2H])c(-c2c3ccccc3c(-c3ccc4c(c3)oc3ccccc34)c3ccccc23)c1[2H] ; [2H]c1c([2H])c(-c2ccccc2)c([2H])c(-c2c3ccccc3c(-c3c([2H])c([2H])c4oc5c([2H])c([2H])c([2H])c([2H])c5c4c3[2H])c3ccccc23)c1[2H] ; [2H]c1c([2H])c(-c2ccccc2)c([2H])c(-c2c3ccccc3c(-c3c([2H])c([2H])c([2H])c4c3oc3c([2H])c([2H])c([2H])c([2H])c34)c3ccccc23)c1[2H]]

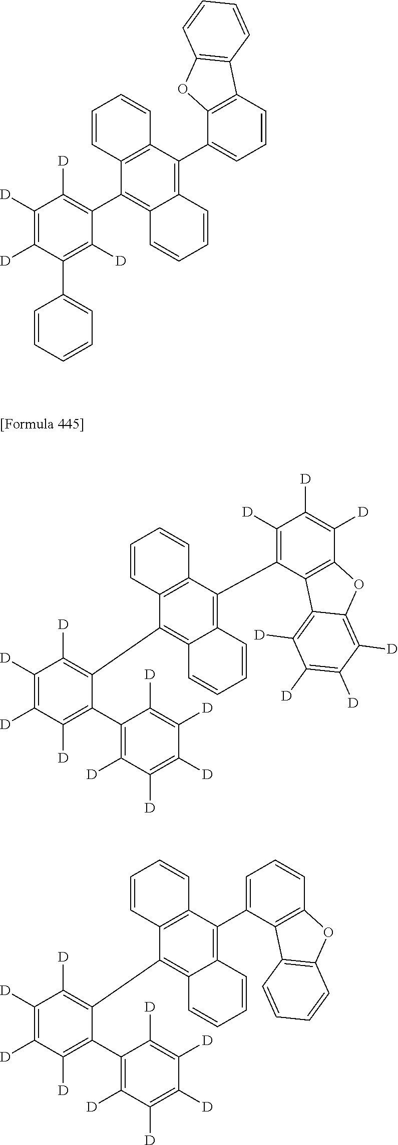 [SMILES: [2H]c1c([2H])c(-c2ccccc2)c([2H])c(-c2c3ccccc3c(-c3cccc4c3oc3ccccc34)c3ccccc23)c1[2H] ; [2H]c1c([2H])c([2H])c(-c2c([2H])c([2H])c([2H])c([2H])c2-c2c3ccccc3c(-c3c([2H])c([2H])c([2H])c4oc5c([2H])c([2H])c([2H])c([2H])c5c34)c3ccccc23)c([2H])c1[2H] ; [2H]c1c([2H])c([2H])c(-c2c([2H])c([2H])c([2H])c([2H])c2-c2c3ccccc3c(-c3cccc4oc5ccccc5c34)c3ccccc23)c([2H])c1[2H]]

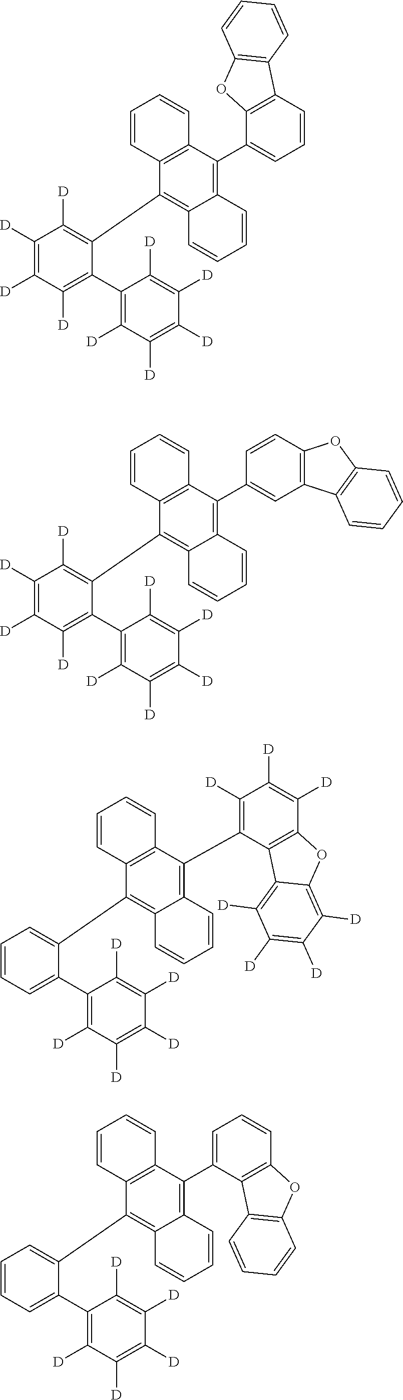

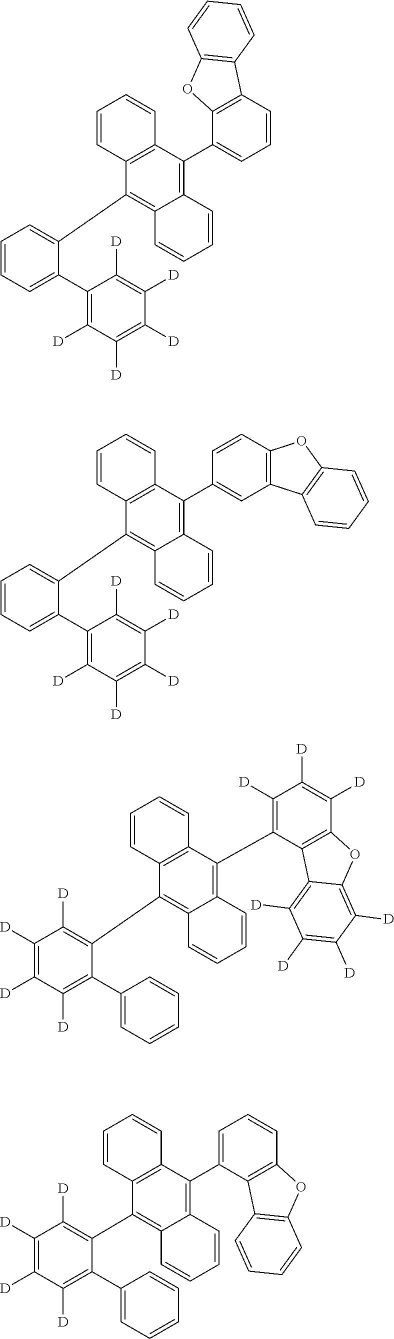 [SMILES: [2H]c1c([2H])c([2H])c(-c2ccccc2-c2c3ccccc3c(-c3cccc4c3oc3ccccc34)c3ccccc23)c([2H])c1[2H] ; [2H]c1c([2H])c([2H])c(-c2ccccc2-c2c3ccccc3c(-c3ccc4oc5ccccc5c4c3)c3ccccc23)c([2H])c1[2H] ; [2H]c1c([2H])c([2H])c(-c2c3ccccc3c(-c3c([2H])c([2H])c([2H])c4oc5c([2H])c([2H])c([2H])c([2H])c5c34)c3ccccc23)c(-c2ccccc2)c1[2H] ; [2H]c1c([2H])c([2H])c(-c2c3ccccc3c(-c3cccc4oc5ccccc5c34)c3ccccc23)c(-c2ccccc2)c1[2H]]

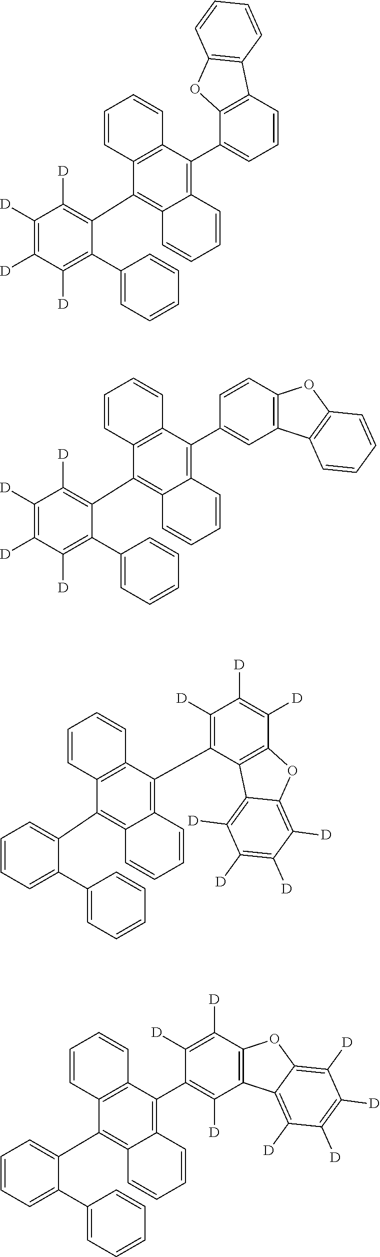 [SMILES: [2H]c1c([2H])c([2H])c(-c2c3ccccc3c(-c3cccc4c3oc3ccccc34)c3ccccc23)c(-c2ccccc2)c1[2H] ; [2H]c1c([2H])c([2H])c(-c2c3ccccc3c(-c3ccc4oc5ccccc5c4c3)c3ccccc23)c(-c2ccccc2)c1[2H] ; [2H]c1c([2H])c([2H])c2c(oc3c([2H])c([2H])c([2H])c(-c4c5ccccc5c(-c5ccccc5-c5ccccc5)c5ccccc45)c32)c1[2H] ; [2H]c1c([2H])c([2H])c2c(oc3c([2H])c([2H])c(-c4c5ccccc5c(-c5ccccc5-c5ccccc5)c5ccccc45)c([2H])c32)c1[2H]]

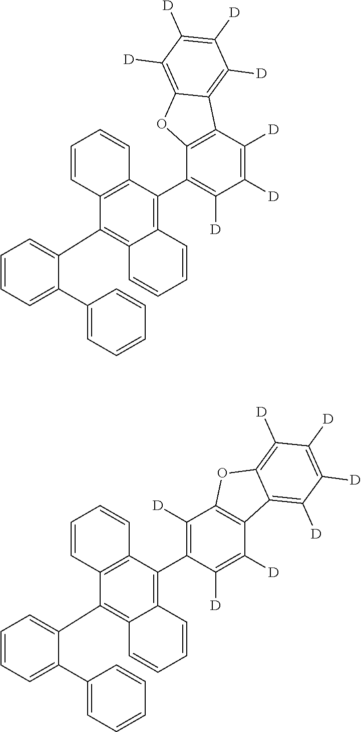 [SMILES: [2H]c1c([2H])c([2H])c2c(oc3c(-c4c5ccccc5c(-c5ccccc5-c5ccccc5)c5ccccc45)c([2H])c([2H])c([2H])c32)c1[2H] ; [2H]c1c([2H])c([2H])c2c(oc3c([2H])c(-c4c5ccccc5c(-c5ccccc5-c5ccccc5)c5ccccc45)c([2H])c([2H])c32)c1[2H]]

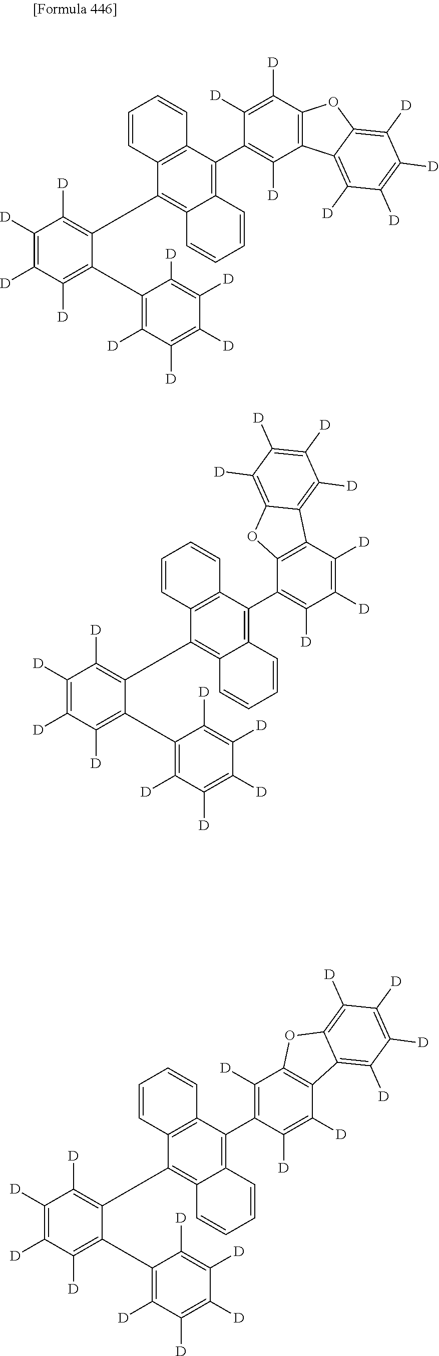 [SMILES: [2H]c1c([2H])c([2H])c(-c2c([2H])c([2H])c([2H])c([2H])c2-c2c3ccccc3c(-c3c([2H])c([2H])c4oc5c([2H])c([2H])c([2H])c([2H])c5c4c3[2H])c3ccccc23)c([2H])c1[2H] ; [2H]c1c([2H])c([2H])c(-c2c([2H])c([2H])c([2H])c([2H])c2-c2c3ccccc3c(-c3c([2H])c([2H])c([2H])c4c3oc3c([2H])c([2H])c([2H])c([2H])c34)c3ccccc23)c([2H])c1[2H] ; [2H]c1c([2H])c([2H])c(-c2c([2H])c([2H])c([2H])c([2H])c2-c2c3ccccc3c(-c3c([2H])c([2H])c4c(oc5c([2H])c([2H])c([2H])c([2H])c54)c3[2H])c3ccccc23)c([2H])c1[2H]]

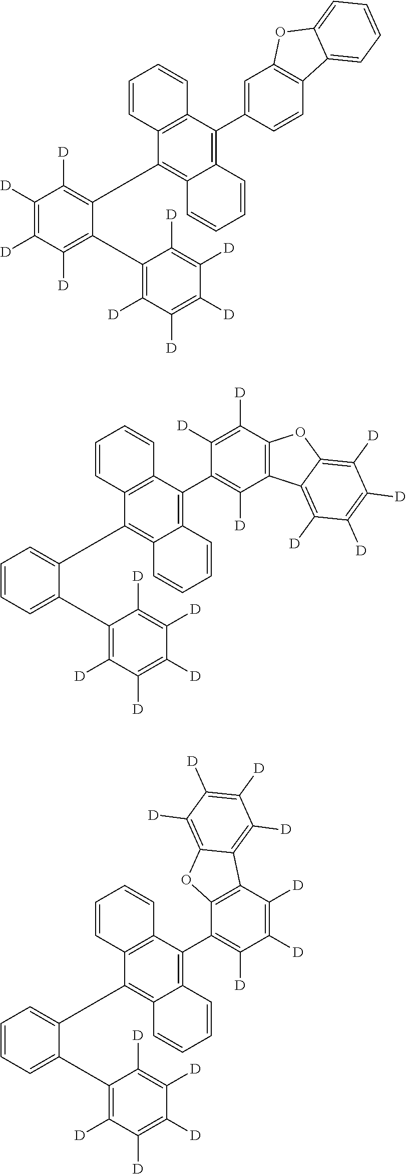 [SMILES: [2H]c1c([2H])c([2H])c(-c2c([2H])c([2H])c([2H])c([2H])c2-c2c3ccccc3c(-c3ccc4c(c3)oc3ccccc34)c3ccccc23)c([2H])c1[2H] ; [2H]c1c([2H])c([2H])c(-c2ccccc2-c2c3ccccc3c(-c3c([2H])c([2H])c4oc5c([2H])c([2H])c([2H])c([2H])c5c4c3[2H])c3ccccc23)c([2H])c1[2H] ; [2H]c1c([2H])c([2H])c(-c2ccccc2-c2c3ccccc3c(-c3c([2H])c([2H])c([2H])c4c3oc3c([2H])c([2H])c([2H])c([2H])c34)c3ccccc23)c([2H])c1[2H]]

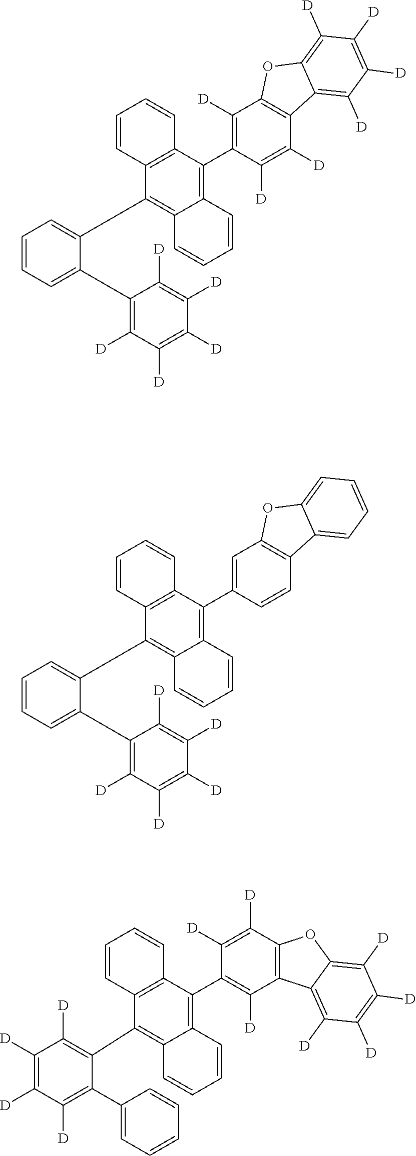 [SMILES: [2H]c1c([2H])c([2H])c(-c2ccccc2-c2c3ccccc3c(-c3c([2H])c([2H])c4c(oc5c([2H])c([2H])c([2H])c([2H])c54)c3[2H])c3ccccc23)c([2H])c1[2H] ; [2H]c1c([2H])c([2H])c(-c2ccccc2-c2c3ccccc3c(-c3ccc4c(c3)oc3ccccc34)c3ccccc23)c([2H])c1[2H] ; [2H]c1c([2H])c([2H])c(-c2c3ccccc3c(-c3c([2H])c([2H])c4oc5c([2H])c([2H])c([2H])c([2H])c5c4c3[2H])c3ccccc23)c(-c2ccccc2)c1[2H]]

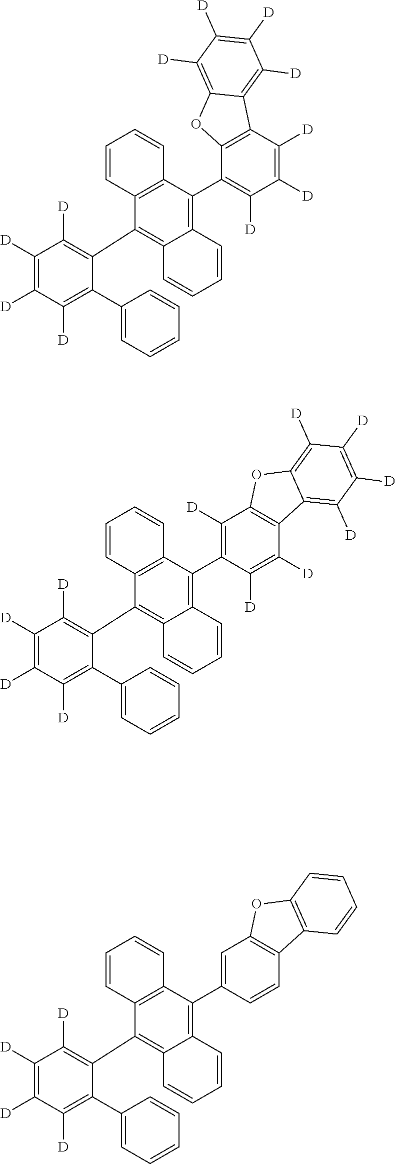 [SMILES: [2H]c1c([2H])c([2H])c(-c2c3ccccc3c(-c3c([2H])c([2H])c([2H])c4c3oc3c([2H])c([2H])c([2H])c([2H])c34)c3ccccc23)c(-c2ccccc2)c1[2H] ; [2H]c1c([2H])c([2H])c(-c2c3ccccc3c(-c3c([2H])c([2H])c4c(oc5c([2H])c([2H])c([2H])c([2H])c54)c3[2H])c3ccccc23)c(-c2ccccc2)c1[2H] ; [2H]c1c([2H])c([2H])c(-c2c3ccccc3c(-c3ccc4c(c3)oc3ccccc34)c3ccccc23)c(-c2ccccc2)c1[2H]]

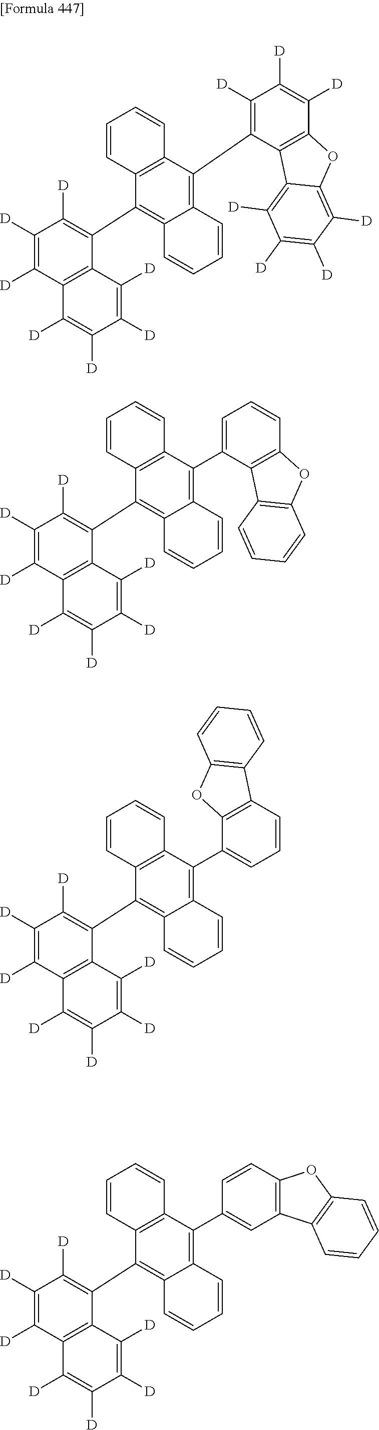 [SMILES: [2H]c1c([2H])c([2H])c2c(oc3c([2H])c([2H])c([2H])c(-c4c5ccccc5c(-c5c([2H])c([2H])c([2H])c6c([2H])c([2H])c([2H])c([2H])c56)c5ccccc45)c32)c1[2H] ; [2H]c1c([2H])c([2H])c2c(-c3c4ccccc4c(-c4cccc5oc6ccccc6c45)c4ccccc34)c([2H])c([2H])c([2H])c2c1[2H] ; [2H]c1c([2H])c([2H])c2c(-c3c4ccccc4c(-c4cccc5c4oc4ccccc45)c4ccccc34)c([2H])c([2H])c([2H])c2c1[2H] ; [2H]c1c([2H])c([2H])c2c(-c3c4ccccc4c(-c4ccc5oc6ccccc6c5c4)c4ccccc34)c([2H])c([2H])c([2H])c2c1[2H]]

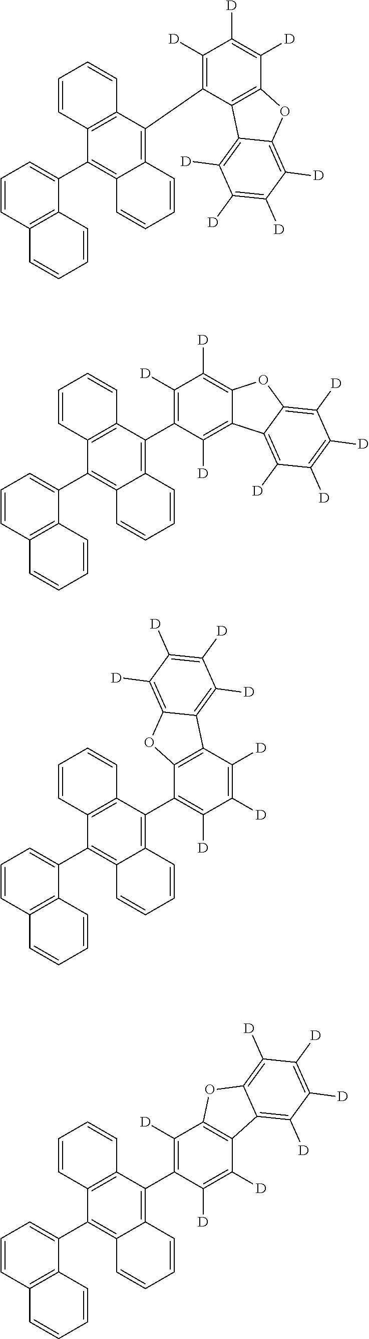 [SMILES: [2H]c1c([2H])c([2H])c2c(oc3c([2H])c([2H])c([2H])c(-c4c5ccccc5c(-c5cccc6ccccc56)c5ccccc45)c32)c1[2H] ; [2H]c1c([2H])c([2H])c2c(oc3c([2H])c([2H])c(-c4c5ccccc5c(-c5cccc6ccccc56)c5ccccc45)c([2H])c32)c1[2H] ; [2H]c1c([2H])c([2H])c2c(oc3c(-c4c5ccccc5c(-c5cccc6ccccc56)c5ccccc45)c([2H])c([2H])c([2H])c32)c1[2H] ; [2H]c1c([2H])c([2H])c2c(oc3c([2H])c(-c4c5ccccc5c(-c5cccc6ccccc56)c5ccccc45)c([2H])c([2H])c32)c1[2H]]

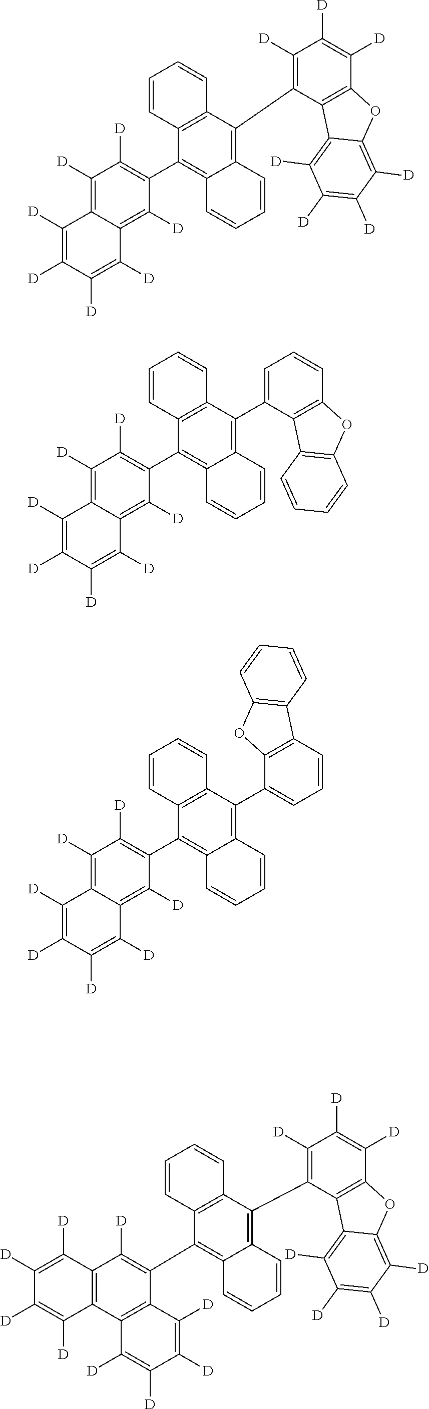

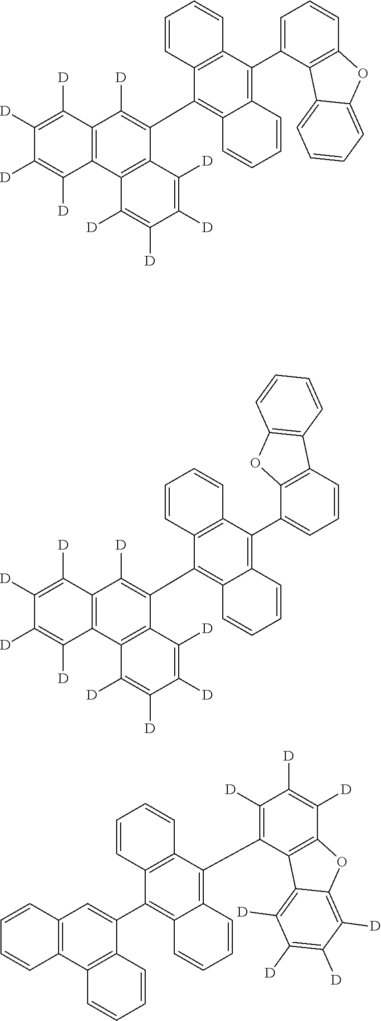 [SMILES: [2H]c1c([2H])c([2H])c2c(c1[2H])c([2H])c(-c1c3ccccc3c(-c3cccc4oc5ccccc5c34)c3ccccc13)c1c([2H])c([2H])c([2H])c([2H])c12 ; [2H]c1c([2H])c([2H])c2c(c1[2H])c([2H])c(-c1c3ccccc3c(-c3cccc4c3oc3ccccc34)c3ccccc13)c1c([2H])c([2H])c([2H])c([2H])c12 ; [2H]c1c([2H])c([2H])c2c(oc3c([2H])c([2H])c([2H])c(-c4c5ccccc5c(-c5cc6ccccc6c6ccccc56)c5ccccc45)c32)c1[2H]]

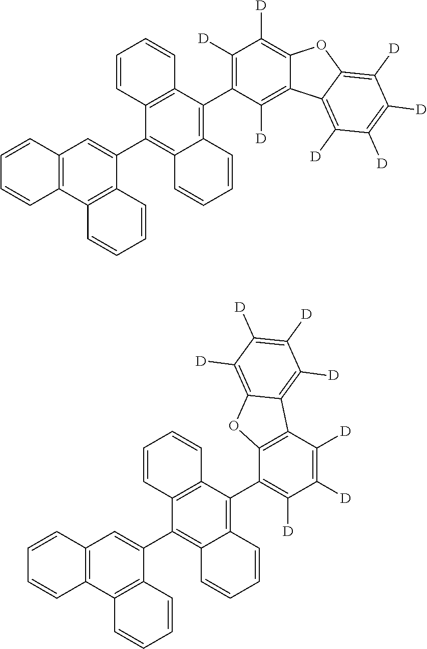 [SMILES: [2H]c1c([2H])c([2H])c2c(oc3c([2H])c([2H])c(-c4c5ccccc5c(-c5cc6ccccc6c6ccccc56)c5ccccc45)c([2H])c32)c1[2H] ; [2H]c1c([2H])c([2H])c2c(oc3c(-c4c5ccccc5c(-c5cc6ccccc6c6ccccc56)c5ccccc45)c([2H])c([2H])c([2H])c32)c1[2H]]

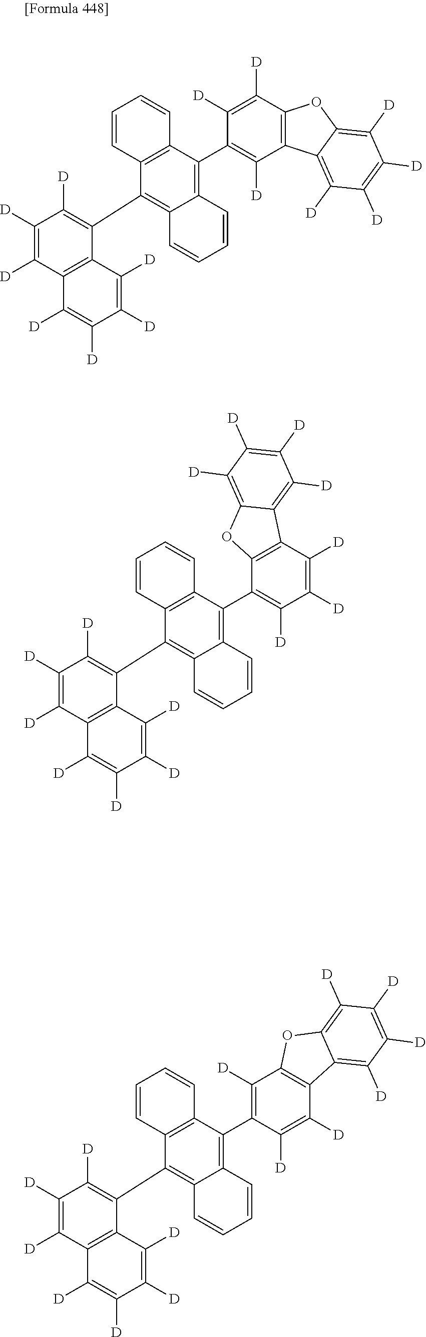 [SMILES: [2H]c1c([2H])c([2H])c2c(oc3c([2H])c([2H])c(-c4c5ccccc5c(-c5c([2H])c([2H])c([2H])c6c([2H])c([2H])c([2H])c([2H])c56)c5ccccc45)c([2H])c32)c1[2H] ; [2H]c1c([2H])c([2H])c2c(oc3c(-c4c5ccccc5c(-c5c([2H])c([2H])c([2H])c6c([2H])c([2H])c([2H])c([2H])c56)c5ccccc45)c([2H])c([2H])c([2H])c32)c1[2H] ; [2H]c1c([2H])c([2H])c2c(oc3c([2H])c(-c4c5ccccc5c(-c5c([2H])c([2H])c([2H])c6c([2H])c([2H])c([2H])c([2H])c56)c5ccccc45)c([2H])c([2H])c32)c1[2H]]

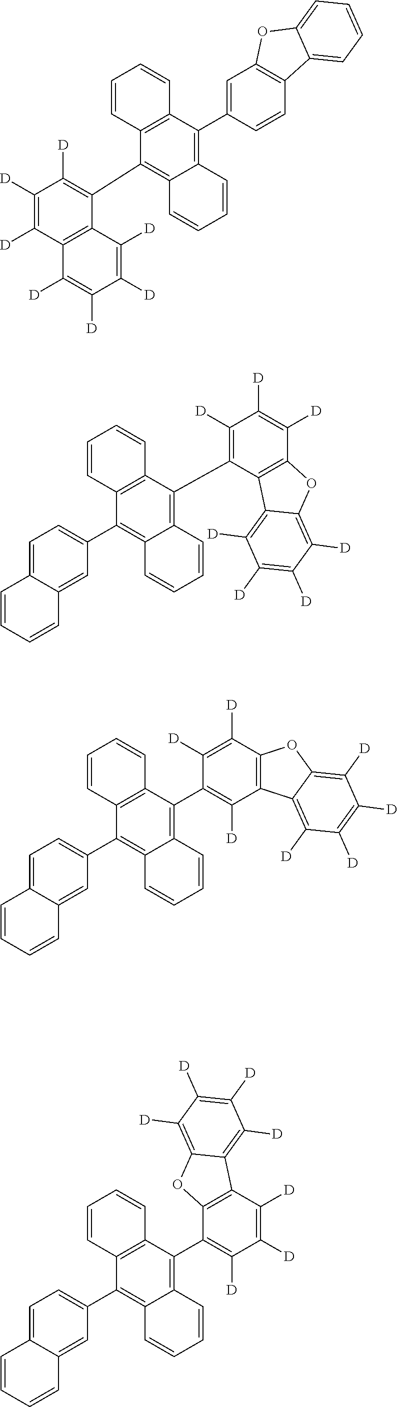 [SMILES: [2H]c1c([2H])c([2H])c2c(-c3c4ccccc4c(-c4ccc5c(c4)oc4ccccc45)c4ccccc34)c([2H])c([2H])c([2H])c2c1[2H] ; [2H]c1c([2H])c([2H])c2c(oc3c([2H])c([2H])c([2H])c(-c4c5ccccc5c(-c5ccc6ccccc6c5)c5ccccc45)c32)c1[2H] ; [2H]c1c([2H])c([2H])c2c(oc3c([2H])c([2H])c(-c4c5ccccc5c(-c5ccc6ccccc6c5)c5ccccc45)c([2H])c32)c1[2H] ; [2H]c1c([2H])c([2H])c2c(oc3c(-c4c5ccccc5c(-c5ccc6ccccc6c5)c5ccccc45)c([2H])c([2H])c([2H])c32)c1[2H]]

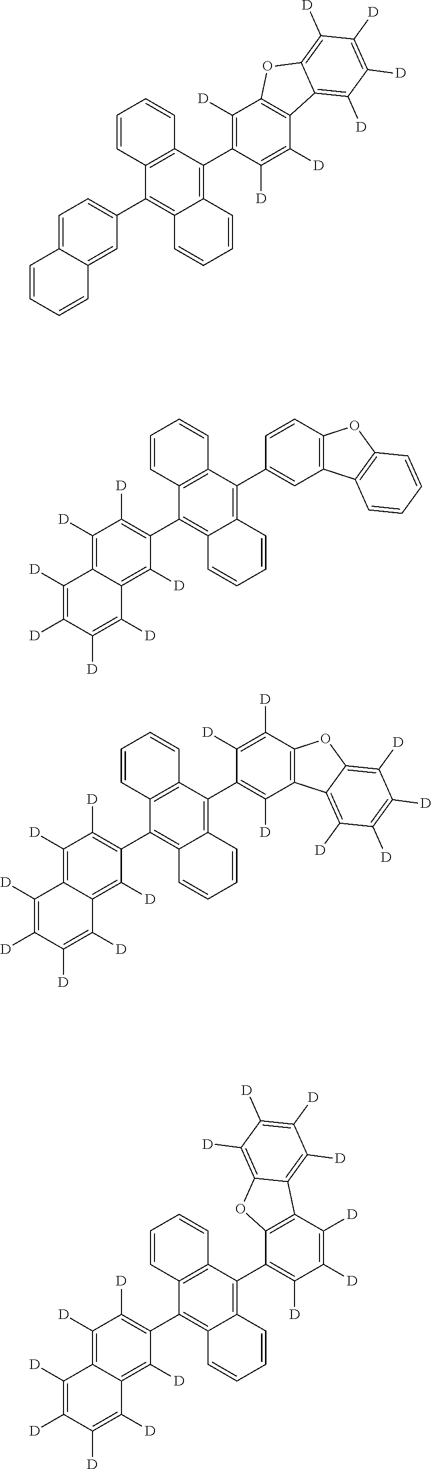 [SMILES: [2H]c1c([2H])c([2H])c2c(oc3c([2H])c(-c4c5ccccc5c(-c5ccc6ccccc6c5)c5ccccc45)c([2H])c([2H])c32)c1[2H] ; [2H]c1c([2H])c([2H])c2c([2H])c(-c3c4ccccc4c(-c4ccc5oc6ccccc6c5c4)c4ccccc34)c([2H])c([2H])c2c1[2H] ; [2H]c1c([2H])c([2H])c2c(oc3c([2H])c([2H])c(-c4c5ccccc5c(-c5c([2H])c([2H])c6c([2H])c([2H])c([2H])c([2H])c6c5[2H])c5ccccc45)c([2H])c32)c1[2H] ; [2H]c1c([2H])c([2H])c2c(oc3c(-c4c5ccccc5c(-c5c([2H])c([2H])c6c([2H])c([2H])c([2H])c([2H])c6c5[2H])c5ccccc45)c([2H])c([2H])c([2H])c32)c1[2H]]

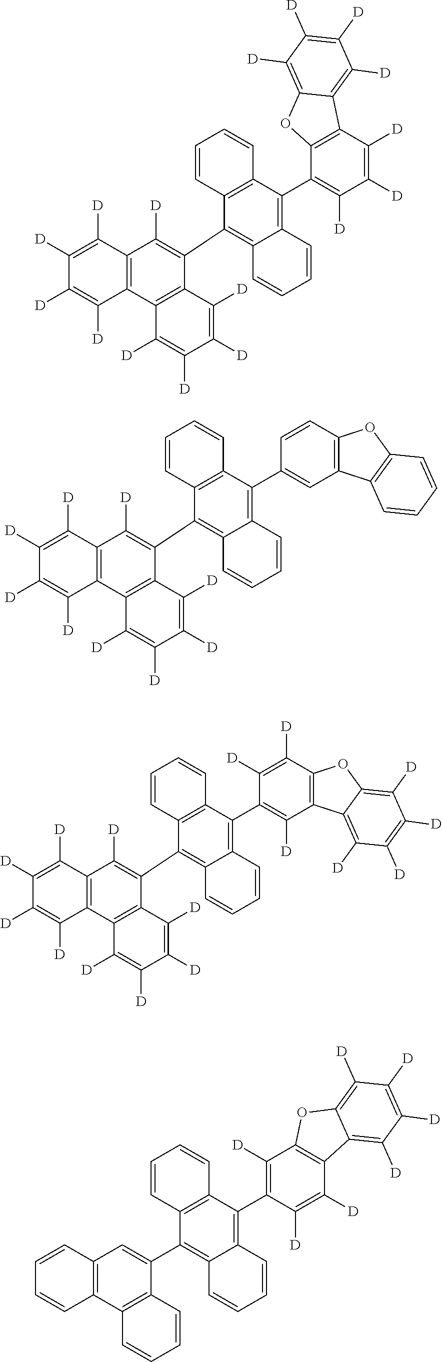

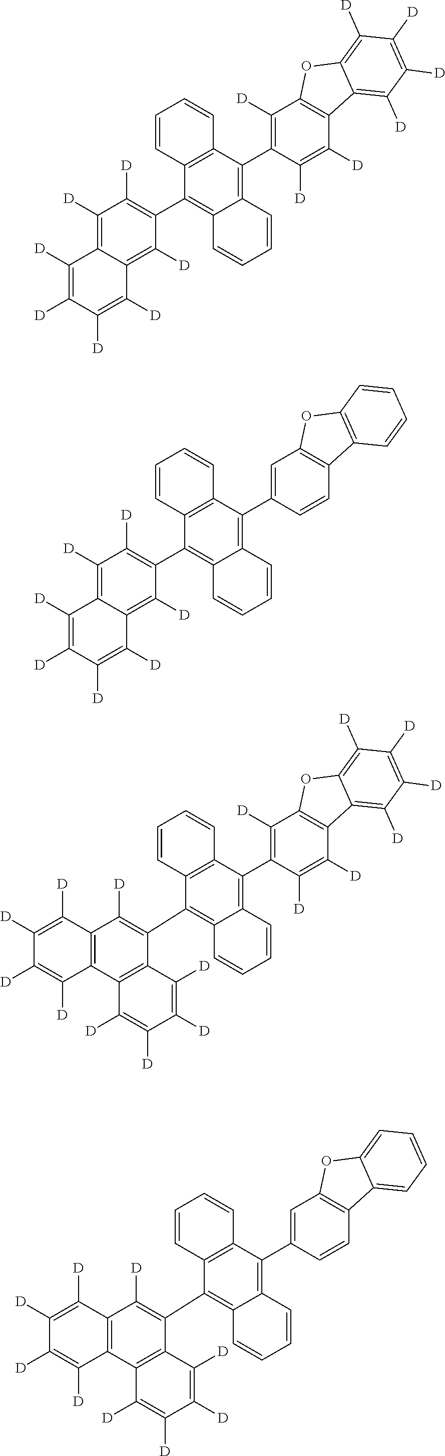

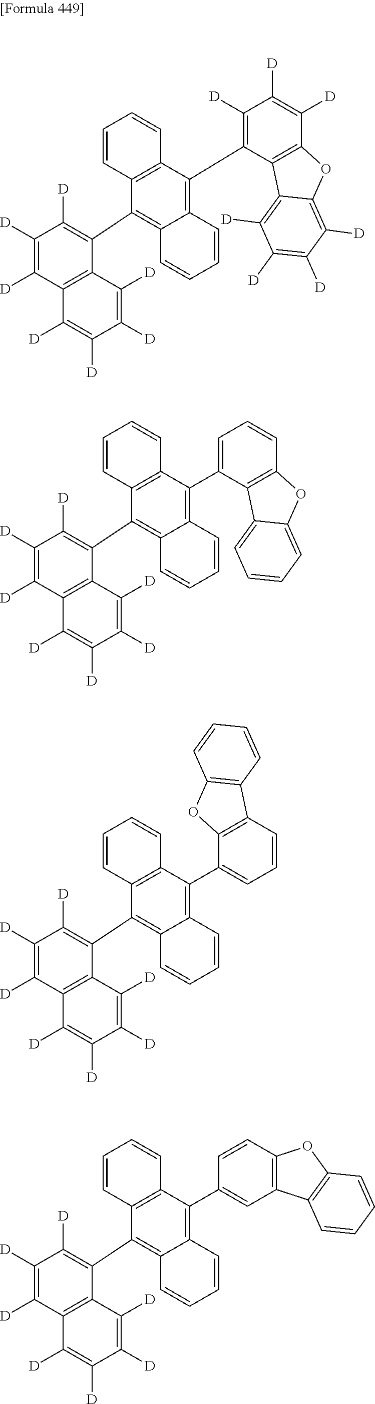 [SMILES: [2H]c1c([2H])c([2H])c2c(oc3c([2H])c([2H])c([2H])c(-c4c5ccccc5c(-c5c([2H])c([2H])c([2H])c6c([2H])c([2H])c([2H])c([2H])c56)c5ccccc45)c32)c1[2H] ; [2H]c1c([2H])c([2H])c2c(-c3c4ccccc4c(-c4cccc5oc6ccccc6c45)c4ccccc34)c([2H])c([2H])c([2H])c2c1[2H] ; [2H]c1c([2H])c([2H])c2c(-c3c4ccccc4c(-c4cccc5c4oc4ccccc45)c4ccccc34)c([2H])c([2H])c([2H])c2c1[2H] ; [2H]c1c([2H])c([2H])c2c(-c3c4ccccc4c(-c4ccc5oc6ccccc6c5c4)c4ccccc34)c([2H])c([2H])c([2H])c2c1[2H]]

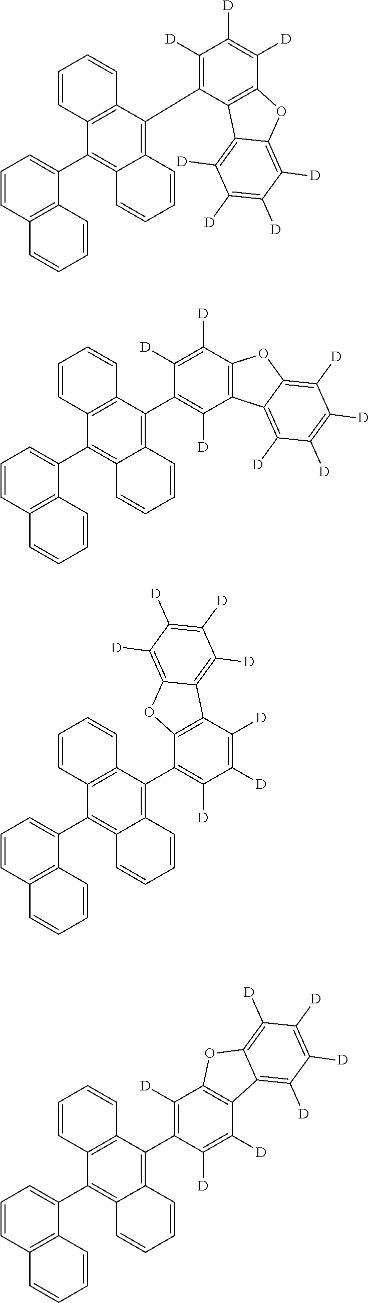 [SMILES: [2H]c1c([2H])c([2H])c2c(oc3c([2H])c([2H])c([2H])c(-c4c5ccccc5c(-c5cccc6ccccc56)c5ccccc45)c32)c1[2H] ; [2H]c1c([2H])c([2H])c2c(oc3c([2H])c([2H])c(-c4c5ccccc5c(-c5cccc6ccccc56)c5ccccc45)c([2H])c32)c1[2H] ; [2H]c1c([2H])c([2H])c2c(oc3c(-c4c5ccccc5c(-c5cccc6ccccc56)c5ccccc45)c([2H])c([2H])c([2H])c32)c1[2H] ; [2H]c1c([2H])c([2H])c2c(oc3c([2H])c(-c4c5ccccc5c(-c5cccc6ccccc56)c5ccccc45)c([2H])c([2H])c32)c1[2H]]

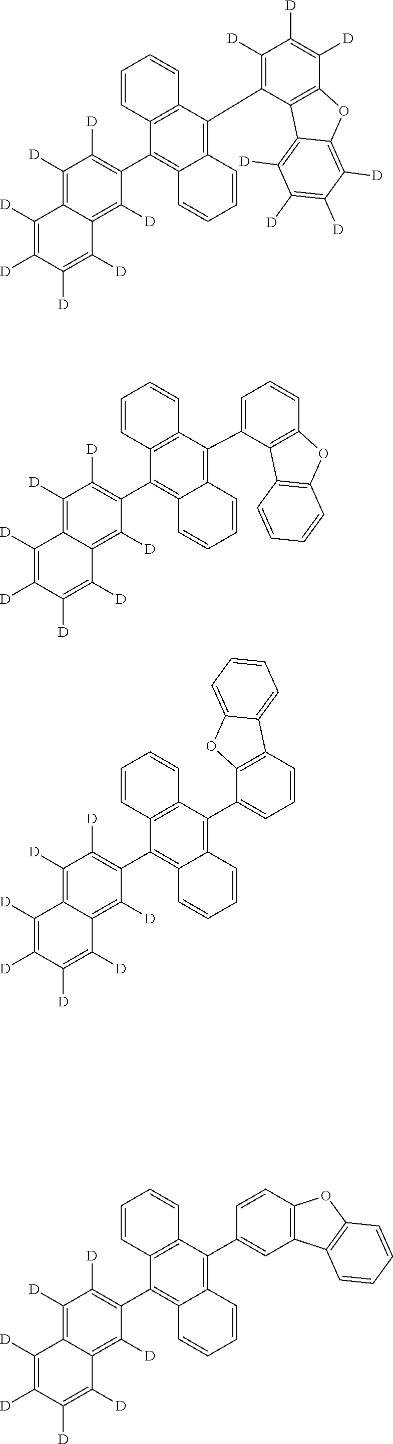 [SMILES: [2H]c1c([2H])c([2H])c2c(oc3c([2H])c([2H])c([2H])c(-c4c5ccccc5c(-c5c([2H])c([2H])c6c([2H])c([2H])c([2H])c([2H])c6c5[2H])c5ccccc45)c32)c1[2H] ; [2H]c1c([2H])c([2H])c2c([2H])c(-c3c4ccccc4c(-c4cccc5oc6ccccc6c45)c4ccccc34)c([2H])c([2H])c2c1[2H] ; [2H]c1c([2H])c([2H])c2c([2H])c(-c3c4ccccc4c(-c4cccc5c4oc4ccccc45)c4ccccc34)c([2H])c([2H])c2c1[2H] ; [2H]c1c([2H])c([2H])c2c([2H])c(-c3c4ccccc4c(-c4ccc5oc6ccccc6c5c4)c4ccccc34)c([2H])c([2H])c2c1[2H]]

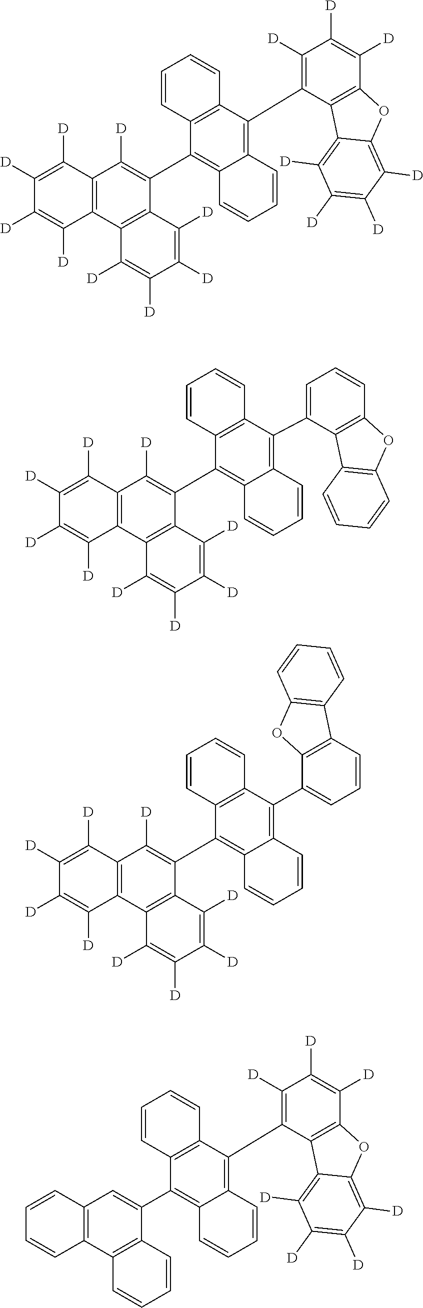

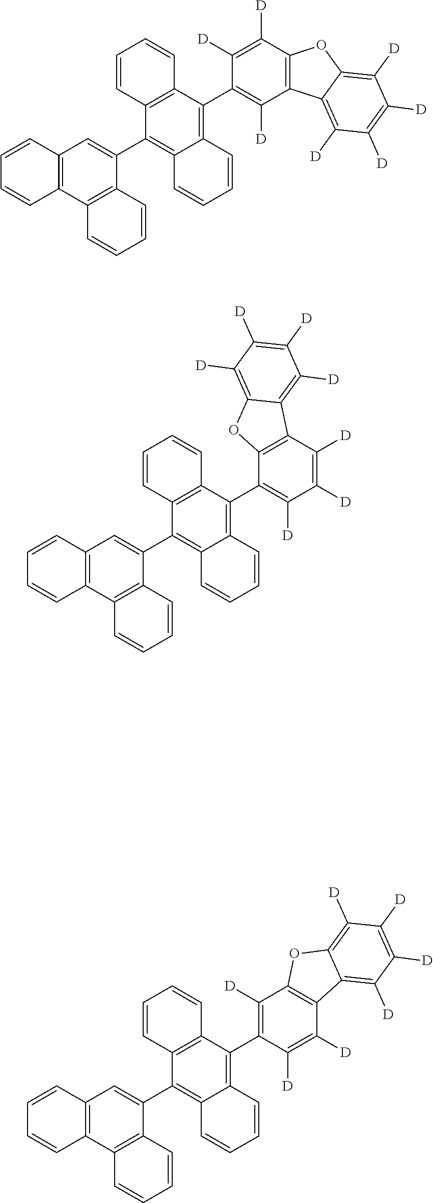

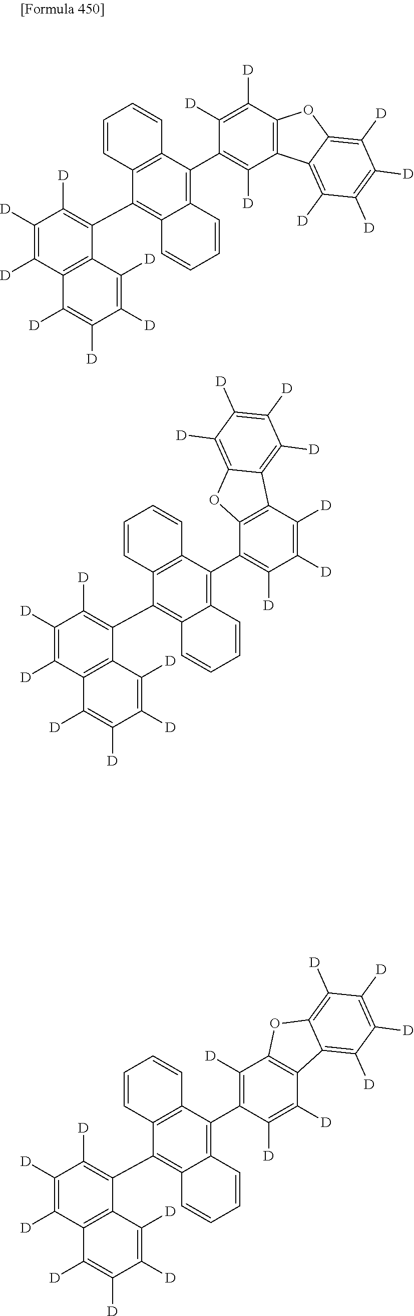

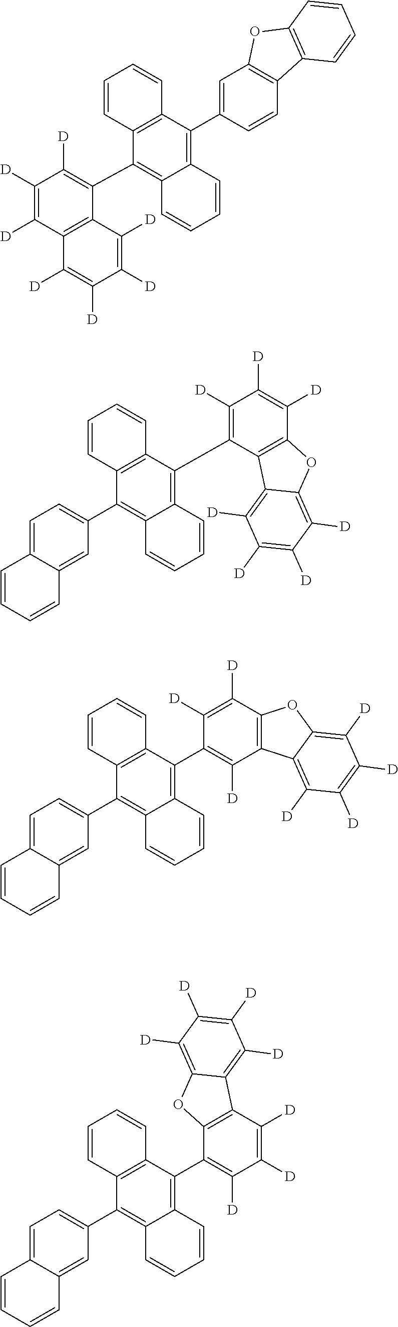

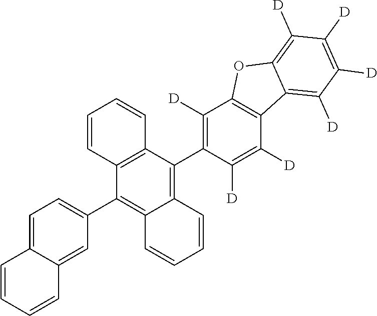

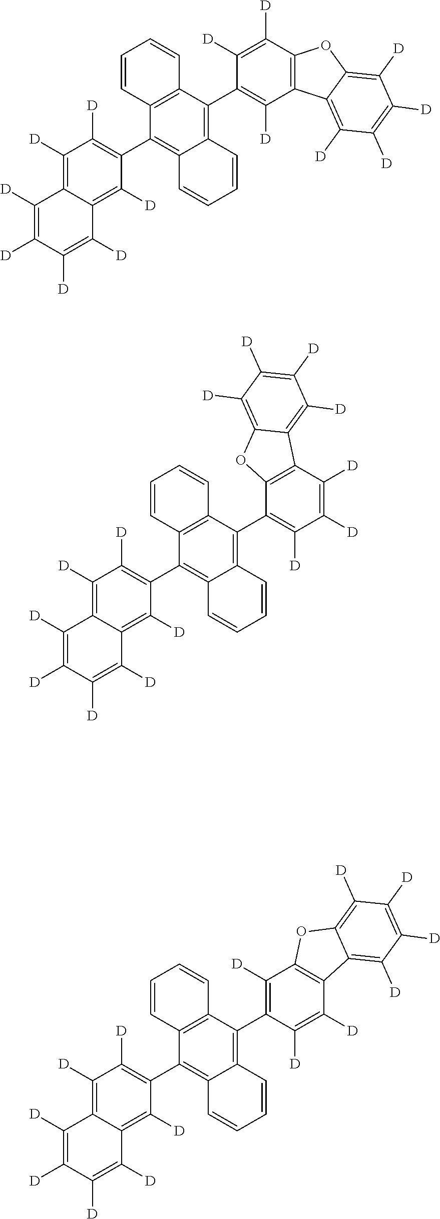

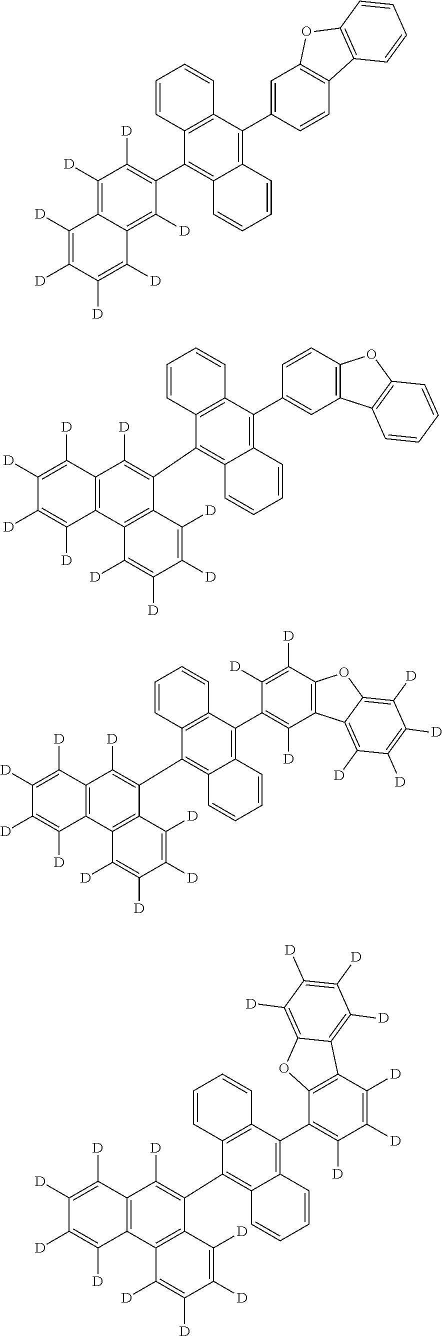

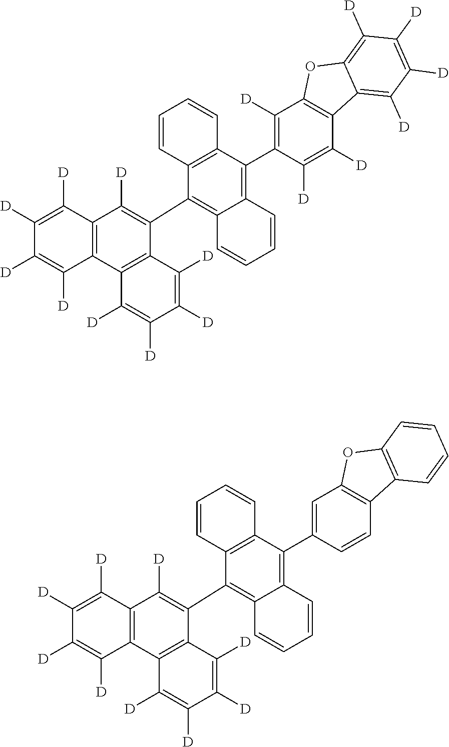 [SMILES: [2H]c1c([2H])c([2H])c2c(oc3c([2H])c(-c4c5ccccc5c(-c5c([2H])c6c([2H])c([2H])c([2H])c([2H])c6c6c([2H])c([2H])c([2H])c([2H])c56)c5ccccc45)c([2H])c([2H])c32)c1[2H] ; [2H]c1c([2H])c([2H])c2c(c1[2H])c([2H])c(-c1c3ccccc3c(-c3ccc4c(c3)oc3ccccc34)c3ccccc13)c1c([2H])c([2H])c([2H])c([2H])c12]

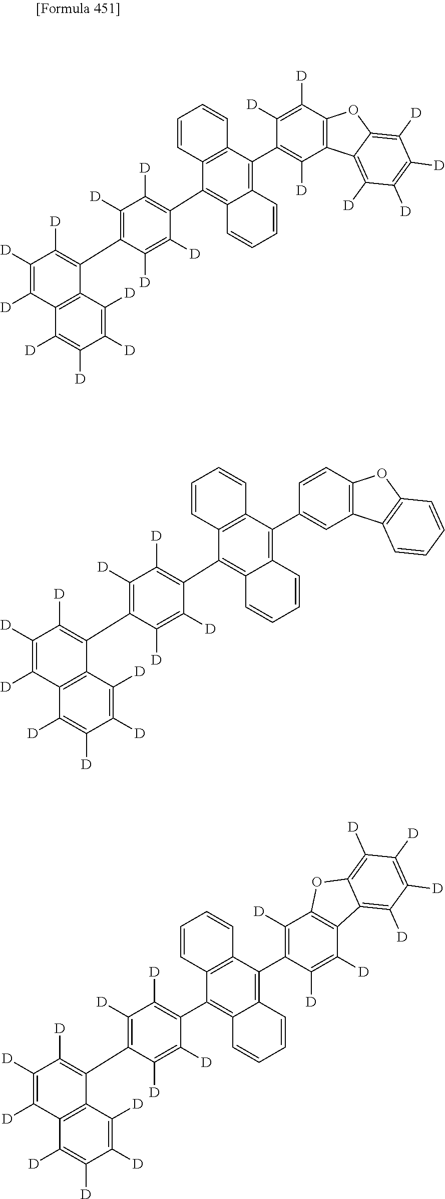 [SMILES: [2H]c1c([2H])c(-c2c([2H])c([2H])c([2H])c3c([2H])c([2H])c([2H])c([2H])c23)c([2H])c([2H])c1-c1c2ccccc2c(-c2c([2H])c([2H])c3oc4c([2H])c([2H])c([2H])c([2H])c4c3c2[2H])c2ccccc12 ; [2H]c1c([2H])c(-c2c([2H])c([2H])c([2H])c3c([2H])c([2H])c([2H])c([2H])c23)c([2H])c([2H])c1-c1c2ccccc2c(-c2ccc3oc4ccccc4c3c2)c2ccccc12 ; [2H]c1c([2H])c(-c2c([2H])c([2H])c([2H])c3c([2H])c([2H])c([2H])c([2H])c23)c([2H])c([2H])c1-c1c2ccccc2c(-c2c([2H])c([2H])c3c(oc4c([2H])c([2H])c([2H])c([2H])c43)c2[2H])c2ccccc12]

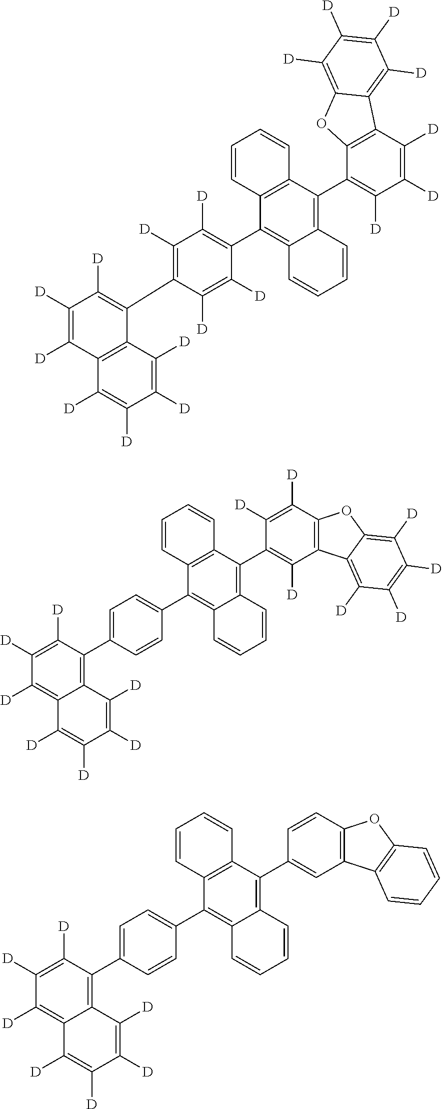 [SMILES: [2H]c1c([2H])c(-c2c([2H])c([2H])c([2H])c3c([2H])c([2H])c([2H])c([2H])c23)c([2H])c([2H])c1-c1c2ccccc2c(-c2c([2H])c([2H])c([2H])c3c2oc2c([2H])c([2H])c([2H])c([2H])c23)c2ccccc12 ; [2H]c1c([2H])c([2H])c2c(oc3c([2H])c([2H])c(-c4c5ccccc5c(-c5ccc(-c6c([2H])c([2H])c([2H])c7c([2H])c([2H])c([2H])c([2H])c67)cc5)c5ccccc45)c([2H])c32)c1[2H] ; [2H]c1c([2H])c([2H])c2c(-c3ccc(-c4c5ccccc5c(-c5ccc6oc7ccccc7c6c5)c5ccccc45)cc3)c([2H])c([2H])c([2H])c2c1[2H]]

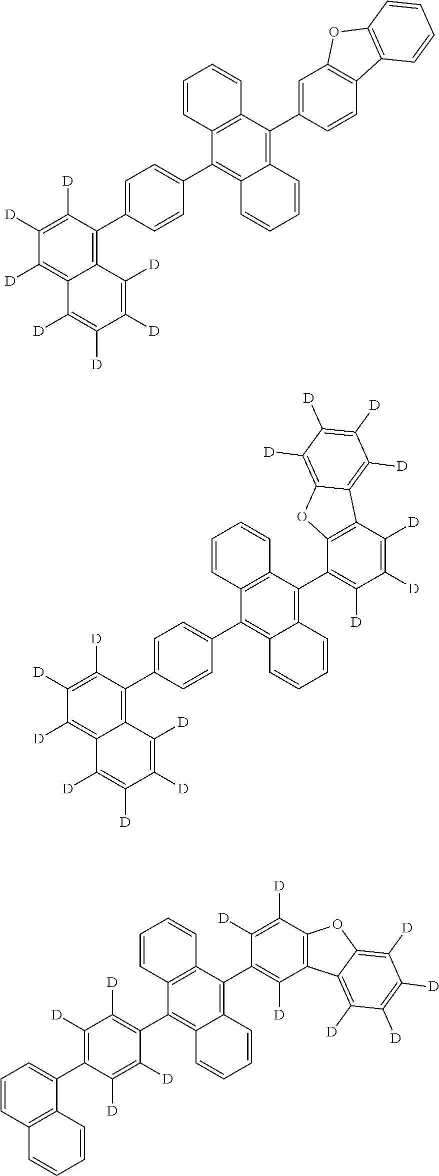 [SMILES: [2H]c1c([2H])c([2H])c2c(-c3ccc(-c4c5ccccc5c(-c5ccc6c(c5)oc5ccccc56)c5ccccc45)cc3)c([2H])c([2H])c([2H])c2c1[2H] ; [2H]c1c([2H])c([2H])c2c(oc3c(-c4c5ccccc5c(-c5ccc(-c6c([2H])c([2H])c([2H])c7c([2H])c([2H])c([2H])c([2H])c67)cc5)c5ccccc45)c([2H])c([2H])c([2H])c32)c1[2H] ; [2H]c1c([2H])c(-c2c3ccccc3c(-c3c([2H])c([2H])c4oc5c([2H])c([2H])c([2H])c([2H])c5c4c3[2H])c3ccccc23)c([2H])c([2H])c1-c1cccc2ccccc12]

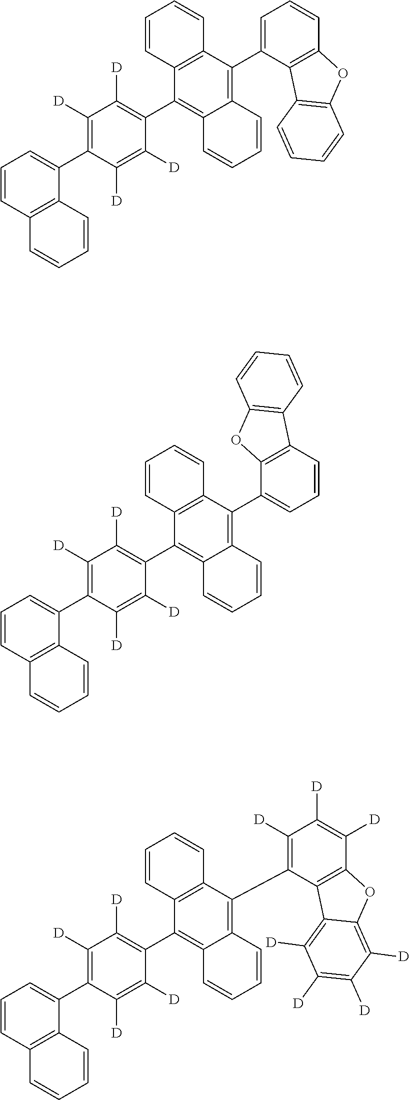 [SMILES: [2H]c1c([2H])c(-c2c3ccccc3c(-c3cccc4oc5ccccc5c34)c3ccccc23)c([2H])c([2H])c1-c1cccc2ccccc12 ; [2H]c1c([2H])c(-c2c3ccccc3c(-c3cccc4c3oc3ccccc34)c3ccccc23)c([2H])c([2H])c1-c1cccc2ccccc12 ; [2H]c1c([2H])c(-c2c3ccccc3c(-c3c([2H])c([2H])c([2H])c4oc5c([2H])c([2H])c([2H])c([2H])c5c34)c3ccccc23)c([2H])c([2H])c1-c1cccc2ccccc12]

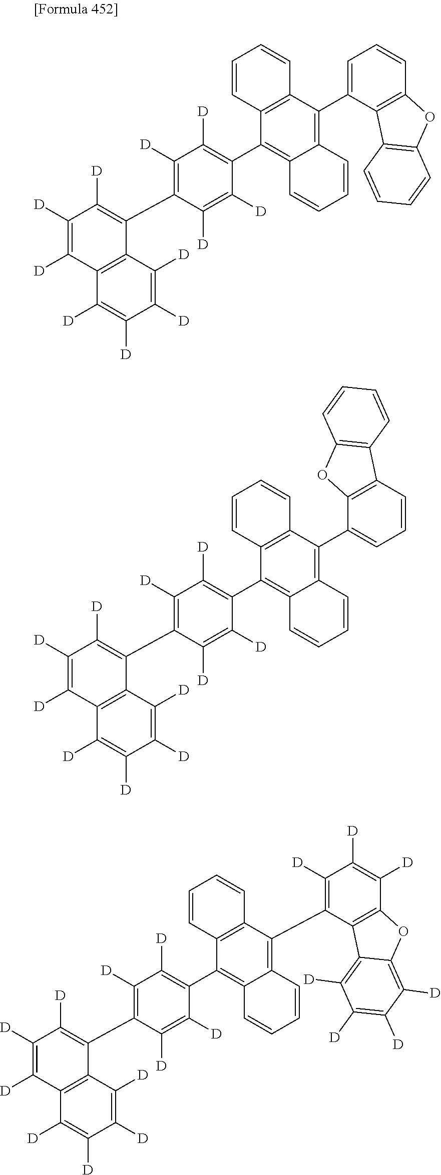 [SMILES: [2H]c1c([2H])c(-c2c([2H])c([2H])c([2H])c3c([2H])c([2H])c([2H])c([2H])c23)c([2H])c([2H])c1-c1c2ccccc2c(-c2cccc3oc4ccccc4c23)c2ccccc12 ; [2H]c1c([2H])c(-c2c([2H])c([2H])c([2H])c3c([2H])c([2H])c([2H])c([2H])c23)c([2H])c([2H])c1-c1c2ccccc2c(-c2cccc3c2oc2ccccc23)c2ccccc12 ; [2H]c1c([2H])c(-c2c([2H])c([2H])c([2H])c3c([2H])c([2H])c([2H])c([2H])c23)c([2H])c([2H])c1-c1c2ccccc2c(-c2c([2H])c([2H])c([2H])c3oc4c([2H])c([2H])c([2H])c([2H])c4c23)c2ccccc12]

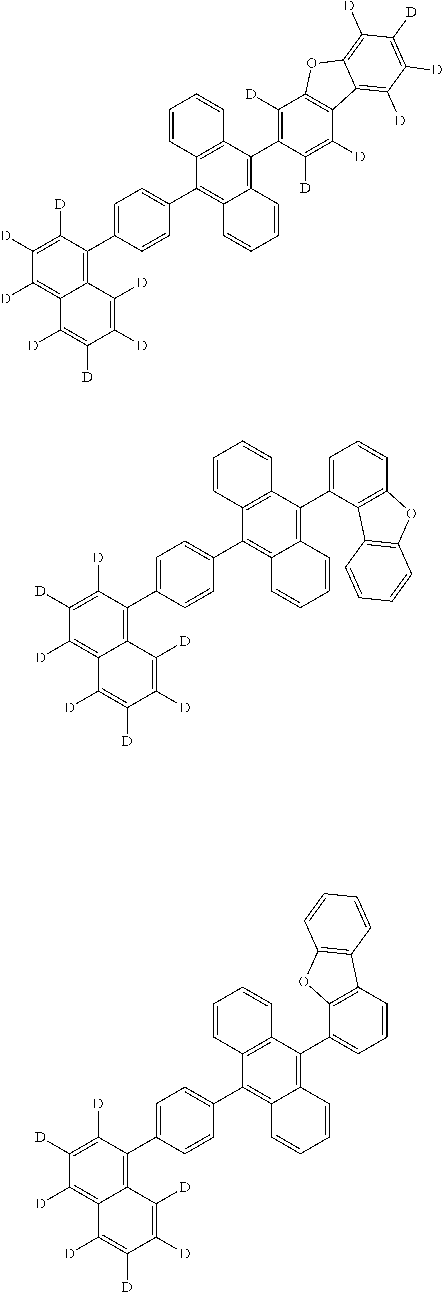 [SMILES: [2H]c1c([2H])c([2H])c2c(oc3c([2H])c(-c4c5ccccc5c(-c5ccc(-c6c([2H])c([2H])c([2H])c7c([2H])c([2H])c([2H])c([2H])c67)cc5)c5ccccc45)c([2H])c([2H])c32)c1[2H] ; [2H]c1c([2H])c([2H])c2c(-c3ccc(-c4c5ccccc5c(-c5cccc6oc7ccccc7c56)c5ccccc45)cc3)c([2H])c([2H])c([2H])c2c1[2H] ; [2H]c1c([2H])c([2H])c2c(-c3ccc(-c4c5ccccc5c(-c5cccc6c5oc5ccccc56)c5ccccc45)cc3)c([2H])c([2H])c([2H])c2c1[2H]]

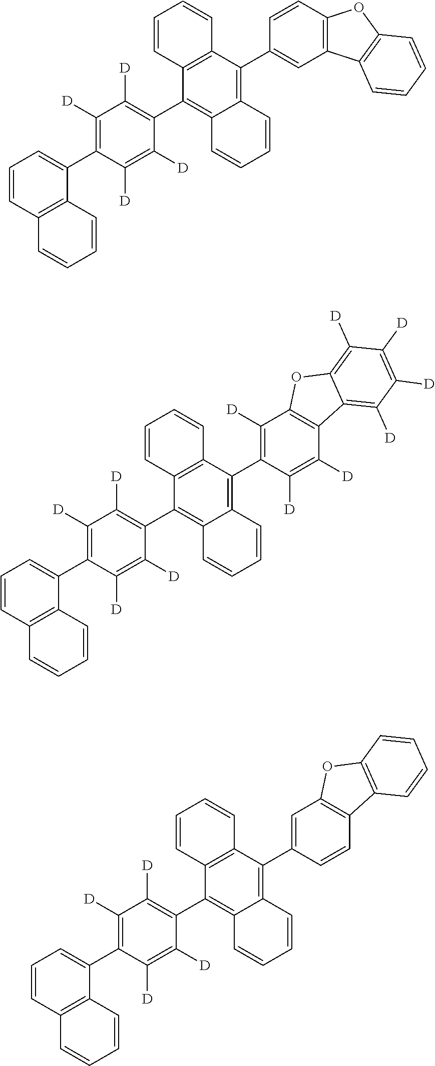 [SMILES: [2H]c1c([2H])c(-c2c3ccccc3c(-c3ccc4oc5ccccc5c4c3)c3ccccc23)c([2H])c([2H])c1-c1cccc2ccccc12 ; [2H]c1c([2H])c(-c2c3ccccc3c(-c3c([2H])c([2H])c4c(oc5c([2H])c([2H])c([2H])c([2H])c54)c3[2H])c3ccccc23)c([2H])c([2H])c1-c1cccc2ccccc12 ; [2H]c1c([2H])c(-c2c3ccccc3c(-c3ccc4c(c3)oc3ccccc34)c3ccccc23)c([2H])c([2H])c1-c1cccc2ccccc12]

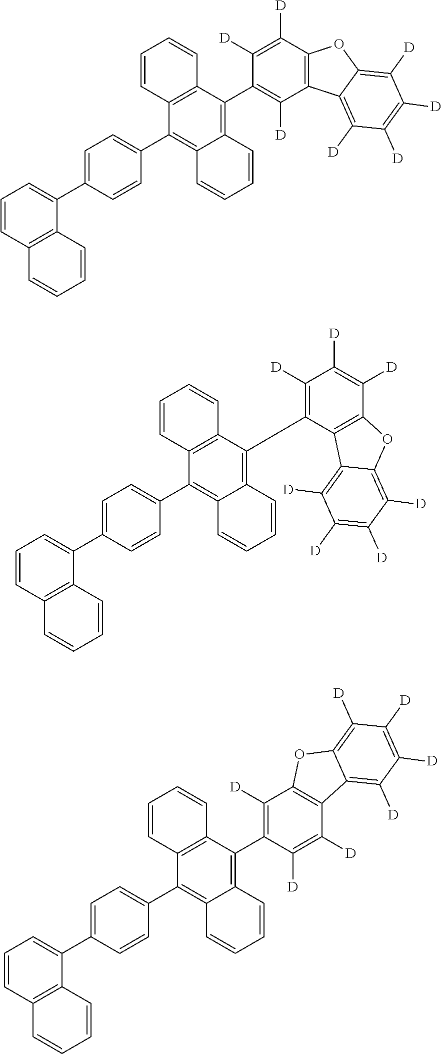 [SMILES: [2H]c1c([2H])c([2H])c2c(oc3c([2H])c([2H])c(-c4c5ccccc5c(-c5ccc(-c6cccc7ccccc67)cc5)c5ccccc45)c([2H])c32)c1[2H] ; [2H]c1c([2H])c([2H])c2c(oc3c([2H])c([2H])c([2H])c(-c4c5ccccc5c(-c5ccc(-c6cccc7ccccc67)cc5)c5ccccc45)c32)c1[2H] ; [2H]c1c([2H])c([2H])c2c(oc3c([2H])c(-c4c5ccccc5c(-c5ccc(-c6cccc7ccccc67)cc5)c5ccccc45)c([2H])c([2H])c32)c1[2H]]

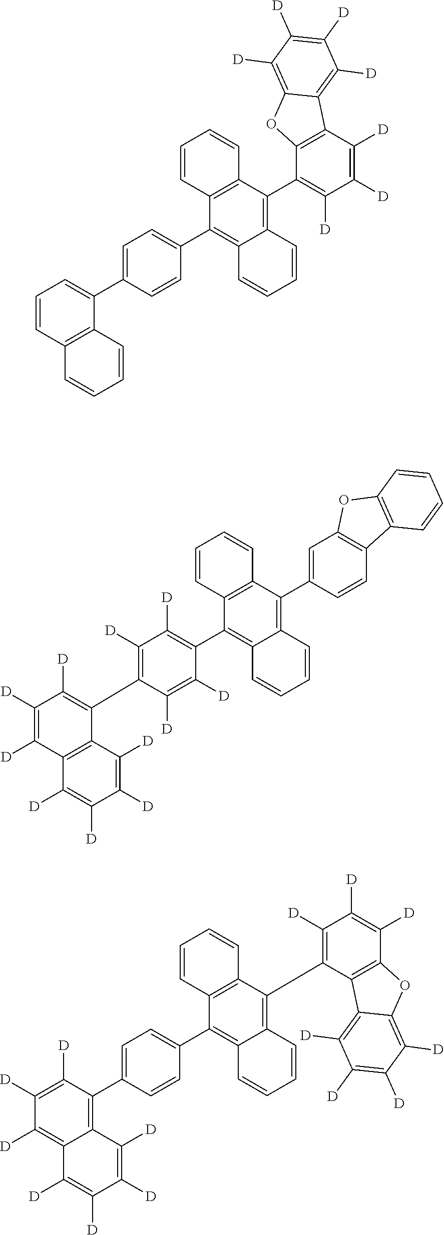 [SMILES: [2H]c1c([2H])c([2H])c2c(oc3c(-c4c5ccccc5c(-c5ccc(-c6cccc7ccccc67)cc5)c5ccccc45)c([2H])c([2H])c([2H])c32)c1[2H] ; [2H]c1c([2H])c(-c2c([2H])c([2H])c([2H])c3c([2H])c([2H])c([2H])c([2H])c23)c([2H])c([2H])c1-c1c2ccccc2c(-c2ccc3c(c2)oc2ccccc23)c2ccccc12 ; [2H]c1c([2H])c([2H])c2c(oc3c([2H])c([2H])c([2H])c(-c4c5ccccc5c(-c5ccc(-c6c([2H])c([2H])c([2H])c7c([2H])c([2H])c([2H])c([2H])c67)cc5)c5ccccc45)c32)c1[2H]]

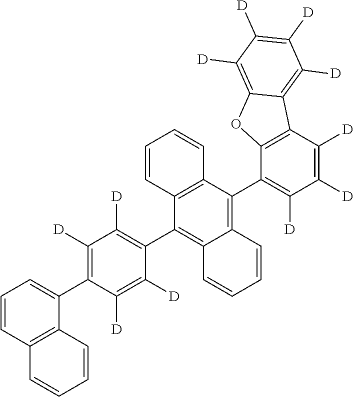 [SMILES: [2H]c1c([2H])c(-c2c3ccccc3c(-c3c([2H])c([2H])c([2H])c4c3oc3c([2H])c([2H])c([2H])c([2H])c34)c3ccccc23)c([2H])c([2H])c1-c1cccc2ccccc12]

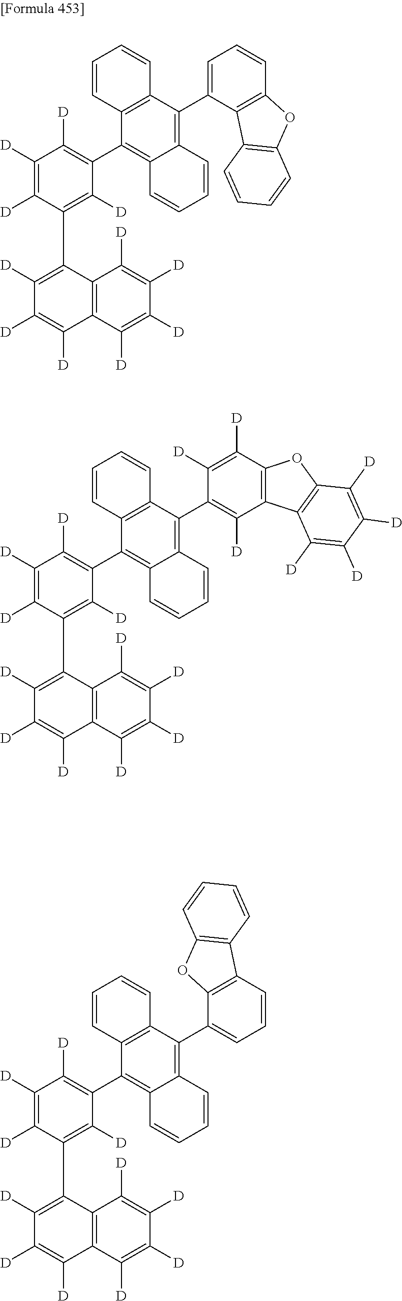 [SMILES: [2H]c1c([2H])c(-c2c3ccccc3c(-c3cccc4oc5ccccc5c34)c3ccccc23)c([2H])c(-c2c([2H])c([2H])c([2H])c3c([2H])c([2H])c([2H])c([2H])c23)c1[2H] ; [2H]c1c([2H])c(-c2c3ccccc3c(-c3c([2H])c([2H])c4oc5c([2H])c([2H])c([2H])c([2H])c5c4c3[2H])c3ccccc23)c([2H])c(-c2c([2H])c([2H])c([2H])c3c([2H])c([2H])c([2H])c([2H])c23)c1[2H] ; [2H]c1c([2H])c(-c2c3ccccc3c(-c3cccc4c3oc3ccccc34)c3ccccc23)c([2H])c(-c2c([2H])c([2H])c([2H])c3c([2H])c([2H])c([2H])c([2H])c23)c1[2H]]

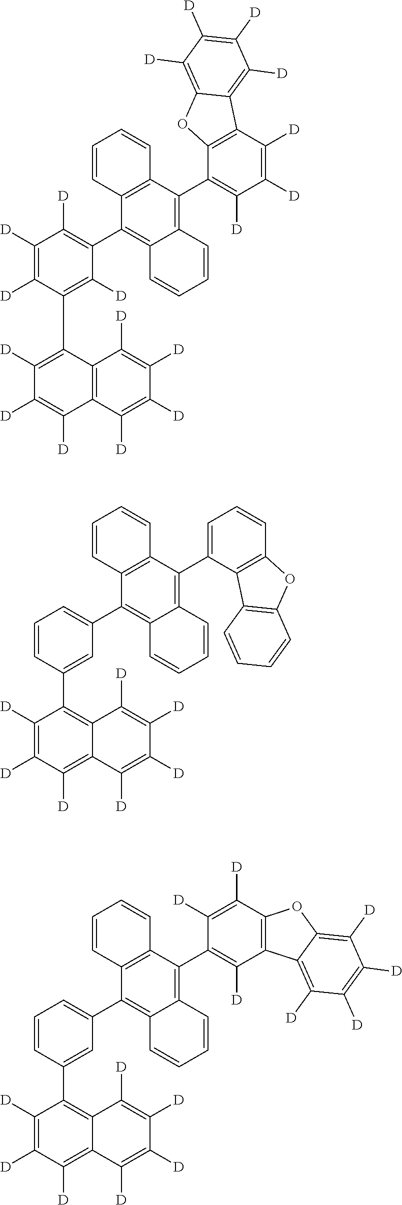 [SMILES: [2H]c1c([2H])c(-c2c3ccccc3c(-c3c([2H])c([2H])c([2H])c4c3oc3c([2H])c([2H])c([2H])c([2H])c34)c3ccccc23)c([2H])c(-c2c([2H])c([2H])c([2H])c3c([2H])c([2H])c([2H])c([2H])c23)c1[2H] ; [2H]c1c([2H])c([2H])c2c(-c3cccc(-c4c5ccccc5c(-c5cccc6oc7ccccc7c56)c5ccccc45)c3)c([2H])c([2H])c([2H])c2c1[2H] ; [2H]c1c([2H])c([2H])c2c(oc3c([2H])c([2H])c(-c4c5ccccc5c(-c5cccc(-c6c([2H])c([2H])c([2H])c7c([2H])c([2H])c([2H])c([2H])c67)c5)c5ccccc45)c([2H])c32)c1[2H]]

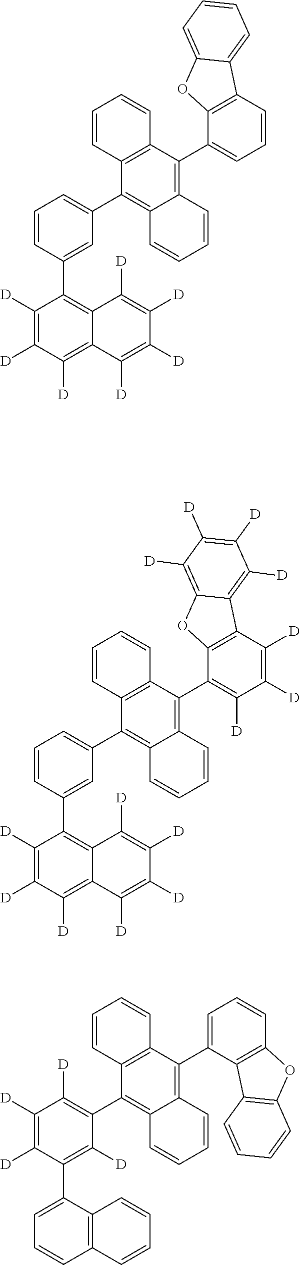 [SMILES: [2H]c1c([2H])c([2H])c2c(-c3cccc(-c4c5ccccc5c(-c5cccc6c5oc5ccccc56)c5ccccc45)c3)c([2H])c([2H])c([2H])c2c1[2H] ; [2H]c1c([2H])c([2H])c2c(oc3c(-c4c5ccccc5c(-c5cccc(-c6c([2H])c([2H])c([2H])c7c([2H])c([2H])c([2H])c([2H])c67)c5)c5ccccc45)c([2H])c([2H])c([2H])c32)c1[2H] ; [2H]c1c([2H])c(-c2cccc3ccccc23)c([2H])c(-c2c3ccccc3c(-c3cccc4oc5ccccc5c34)c3ccccc23)c1[2H]]

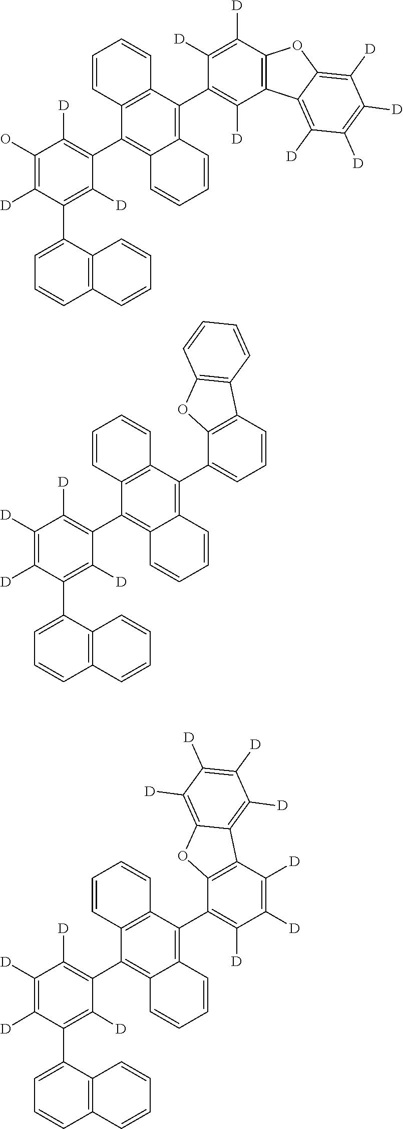

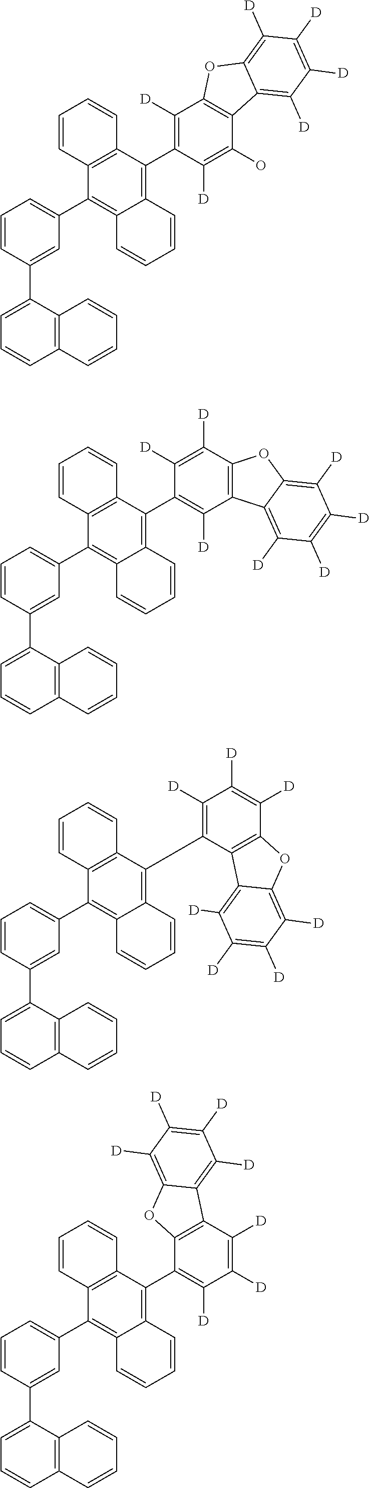

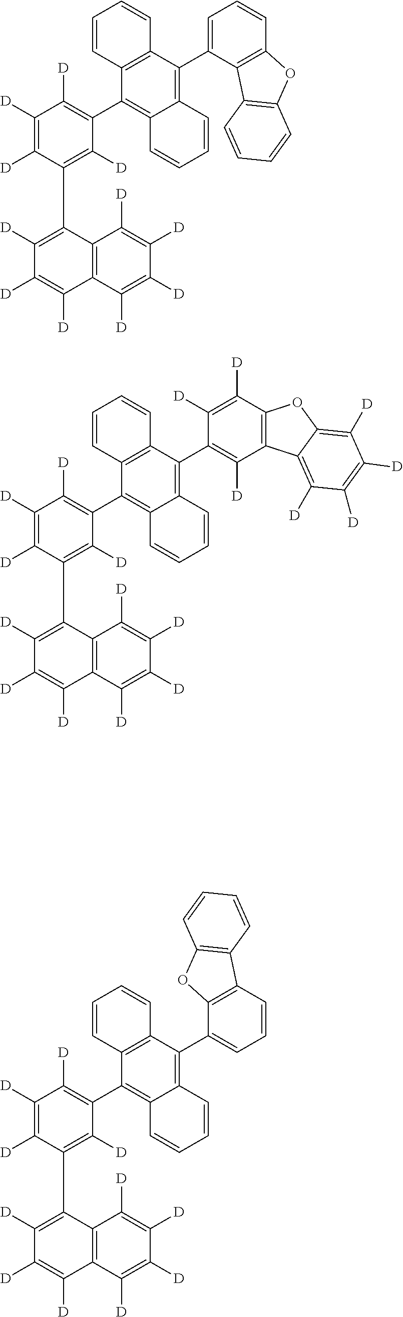 [SMILES: [2H]c1c([2H])c(-c2c3ccccc3c(-c3cccc4oc5ccccc5c34)c3ccccc23)c([2H])c(-c2c([2H])c([2H])c([2H])c3c([2H])c([2H])c([2H])c([2H])c23)c1[2H] ; [2H]c1c([2H])c(-c2c3ccccc3c(-c3c([2H])c([2H])c4oc5c([2H])c([2H])c([2H])c([2H])c5c4c3[2H])c3ccccc23)c([2H])c(-c2c([2H])c([2H])c([2H])c3c([2H])c([2H])c([2H])c([2H])c23)c1[2H] ; [2H]c1c([2H])c(-c2c3ccccc3c(-c3cccc4c3oc3ccccc34)c3ccccc23)c([2H])c(-c2c([2H])c([2H])c([2H])c3c([2H])c([2H])c([2H])c([2H])c23)c1[2H]]

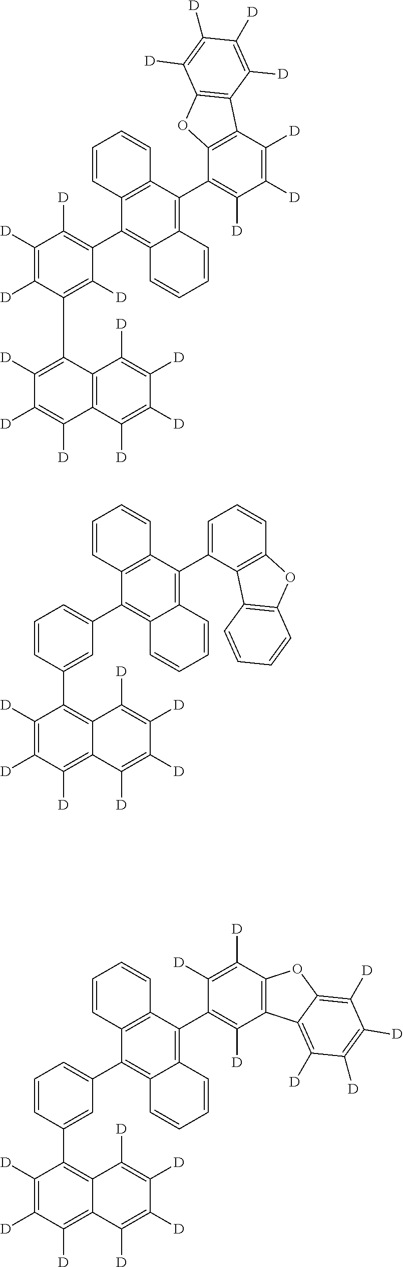 [SMILES: [2H]c1c([2H])c(-c2c3ccccc3c(-c3c([2H])c([2H])c([2H])c4c3oc3c([2H])c([2H])c([2H])c([2H])c34)c3ccccc23)c([2H])c(-c2c([2H])c([2H])c([2H])c3c([2H])c([2H])c([2H])c([2H])c23)c1[2H] ; [2H]c1c([2H])c([2H])c2c(-c3cccc(-c4c5ccccc5c(-c5cccc6oc7ccccc7c56)c5ccccc45)c3)c([2H])c([2H])c([2H])c2c1[2H] ; [2H]c1c([2H])c([2H])c2c(oc3c([2H])c([2H])c(-c4c5ccccc5c(-c5cccc(-c6c([2H])c([2H])c([2H])c7c([2H])c([2H])c([2H])c([2H])c67)c5)c5ccccc45)c([2H])c32)c1[2H]]

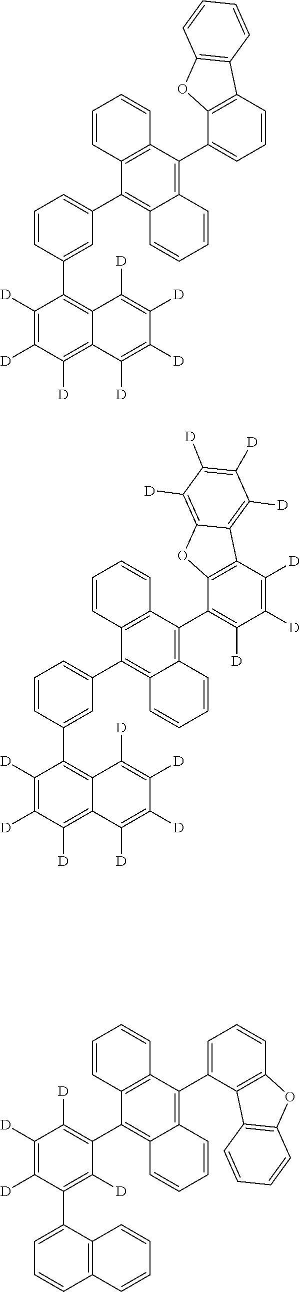 [SMILES: [2H]c1c([2H])c([2H])c2c(-c3cccc(-c4c5ccccc5c(-c5cccc6c5oc5ccccc56)c5ccccc45)c3)c([2H])c([2H])c([2H])c2c1[2H] ; [2H]c1c([2H])c([2H])c2c(oc3c(-c4c5ccccc5c(-c5cccc(-c6c([2H])c([2H])c([2H])c7c([2H])c([2H])c([2H])c([2H])c67)c5)c5ccccc45)c([2H])c([2H])c([2H])c32)c1[2H] ; [2H]c1c([2H])c(-c2cccc3ccccc23)c([2H])c(-c2c3ccccc3c(-c3cccc4oc5ccccc5c34)c3ccccc23)c1[2H]]

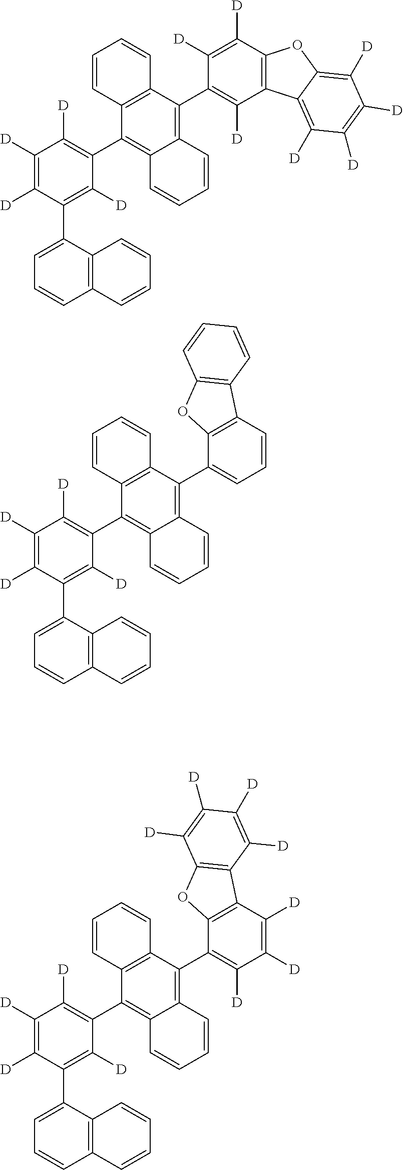 [SMILES: [2H]c1c([2H])c(-c2cccc3ccccc23)c([2H])c(-c2c3ccccc3c(-c3c([2H])c([2H])c4oc5c([2H])c([2H])c([2H])c([2H])c5c4c3[2H])c3ccccc23)c1[2H] ; [2H]c1c([2H])c(-c2cccc3ccccc23)c([2H])c(-c2c3ccccc3c(-c3cccc4c3oc3ccccc34)c3ccccc23)c1[2H] ; [2H]c1c([2H])c(-c2cccc3ccccc23)c([2H])c(-c2c3ccccc3c(-c3c([2H])c([2H])c([2H])c4c3oc3c([2H])c([2H])c([2H])c([2H])c34)c3ccccc23)c1[2H]]

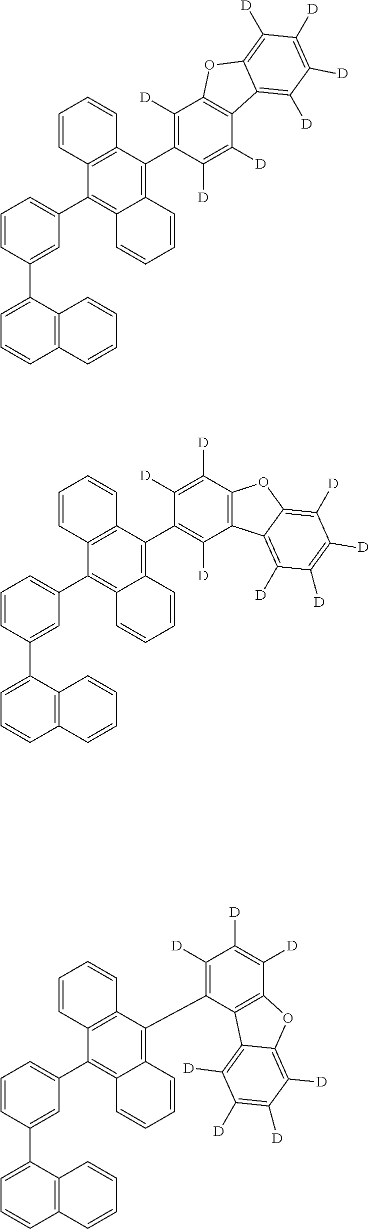 [SMILES: [2H]c1c([2H])c([2H])c2c(oc3c([2H])c(-c4c5ccccc5c(-c5cccc(-c6cccc7ccccc67)c5)c5ccccc45)c([2H])c([2H])c32)c1[2H] ; [2H]c1c([2H])c([2H])c2c(oc3c([2H])c([2H])c(-c4c5ccccc5c(-c5cccc(-c6cccc7ccccc67)c5)c5ccccc45)c([2H])c32)c1[2H] ; [2H]c1c([2H])c([2H])c2c(oc3c([2H])c([2H])c([2H])c(-c4c5ccccc5c(-c5cccc(-c6cccc7ccccc67)c5)c5ccccc45)c32)c1[2H]]

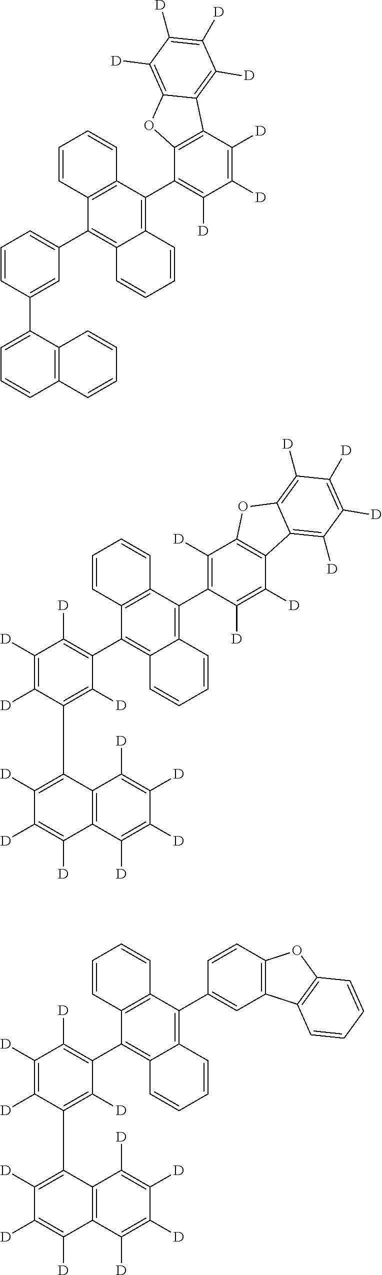 [SMILES: [2H]c1c([2H])c([2H])c2c(oc3c(-c4c5ccccc5c(-c5cccc(-c6cccc7ccccc67)c5)c5ccccc45)c([2H])c([2H])c([2H])c32)c1[2H] ; [2H]c1c([2H])c(-c2c3ccccc3c(-c3c([2H])c([2H])c4c(oc5c([2H])c([2H])c([2H])c([2H])c54)c3[2H])c3ccccc23)c([2H])c(-c2c([2H])c([2H])c([2H])c3c([2H])c([2H])c([2H])c([2H])c23)c1[2H] ; [2H]c1c([2H])c(-c2c3ccccc3c(-c3ccc4oc5ccccc5c4c3)c3ccccc23)c([2H])c(-c2c([2H])c([2H])c([2H])c3c([2H])c([2H])c([2H])c([2H])c23)c1[2H]]

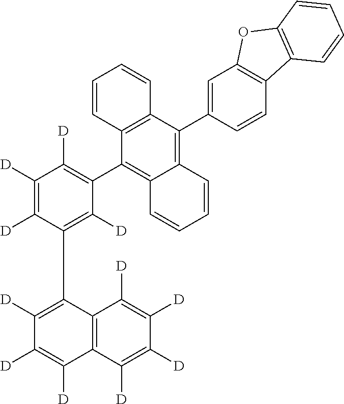 [SMILES: [2H]c1c([2H])c(-c2c3ccccc3c(-c3ccc4c(c3)oc3ccccc34)c3ccccc23)c([2H])c(-c2c([2H])c([2H])c([2H])c3c([2H])c([2H])c([2H])c([2H])c23)c1[2H]]

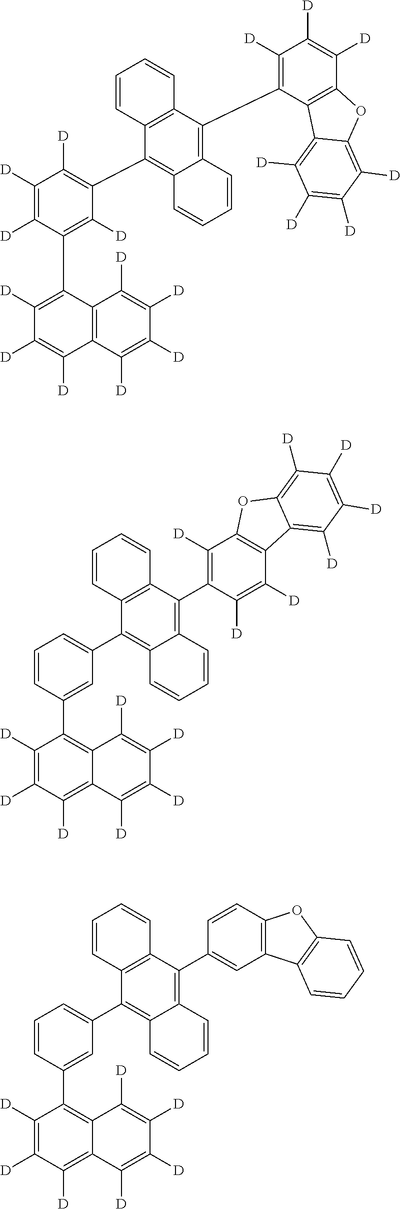 [SMILES: [2H]c1c([2H])c(-c2c3ccccc3c(-c3c([2H])c([2H])c([2H])c4oc5c([2H])c([2H])c([2H])c([2H])c5c34)c3ccccc23)c([2H])c(-c2c([2H])c([2H])c([2H])c3c([2H])c([2H])c([2H])c([2H])c23)c1[2H] ; [2H]c1c([2H])c([2H])c2c(oc3c([2H])c(-c4c5ccccc5c(-c5cccc(-c6c([2H])c([2H])c([2H])c7c([2H])c([2H])c([2H])c([2H])c67)c5)c5ccccc45)c([2H])c([2H])c32)c1[2H] ; [2H]c1c([2H])c([2H])c2c(-c3cccc(-c4c5ccccc5c(-c5ccc6oc7ccccc7c6c5)c5ccccc45)c3)c([2H])c([2H])c([2H])c2c1[2H]]

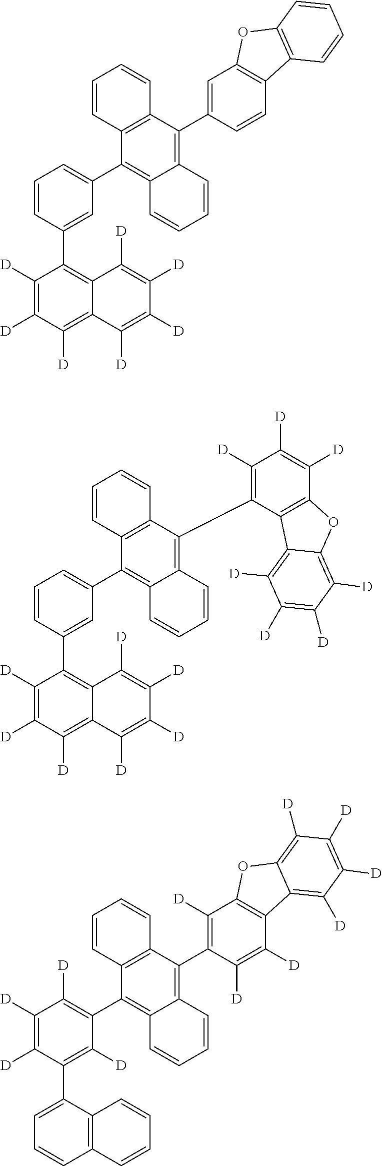 [SMILES: [2H]c1c([2H])c([2H])c2c(-c3cccc(-c4c5ccccc5c(-c5ccc6c(c5)oc5ccccc56)c5ccccc45)c3)c([2H])c([2H])c([2H])c2c1[2H] ; [2H]c1c([2H])c([2H])c2c(oc3c([2H])c([2H])c([2H])c(-c4c5ccccc5c(-c5cccc(-c6c([2H])c([2H])c([2H])c7c([2H])c([2H])c([2H])c([2H])c67)c5)c5ccccc45)c32)c1[2H] ; [2H]c1c([2H])c(-c2cccc3ccccc23)c([2H])c(-c2c3ccccc3c(-c3c([2H])c([2H])c4c(oc5c([2H])c([2H])c([2H])c([2H])c54)c3[2H])c3ccccc23)c1[2H]]

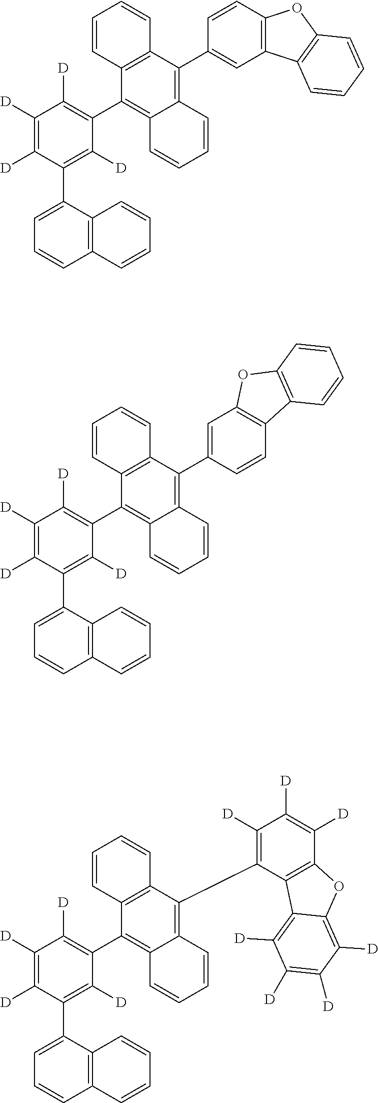 [SMILES: [2H]c1c([2H])c(-c2cccc3ccccc23)c([2H])c(-c2c3ccccc3c(-c3ccc4oc5ccccc5c4c3)c3ccccc23)c1[2H] ; [2H]c1c([2H])c(-c2cccc3ccccc23)c([2H])c(-c2c3ccccc3c(-c3ccc4c(c3)oc3ccccc34)c3ccccc23)c1[2H] ; [2H]c1c([2H])c(-c2cccc3ccccc23)c([2H])c(-c2c3ccccc3c(-c3c([2H])c([2H])c([2H])c4oc5c([2H])c([2H])c([2H])c([2H])c5c34)c3ccccc23)c1[2H]]

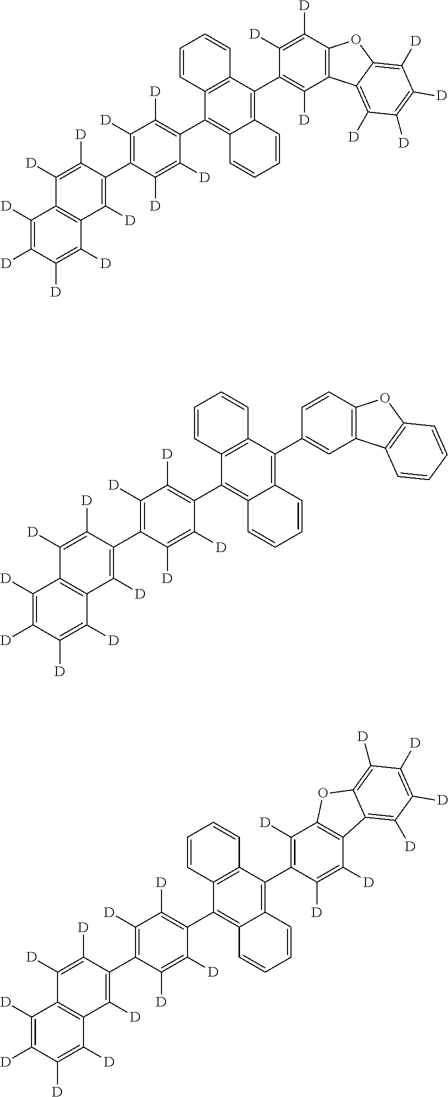 [SMILES: [2H]c1c([2H])c(-c2c3ccccc3c(-c3c([2H])c([2H])c4oc5c([2H])c([2H])c([2H])c([2H])c5c4c3[2H])c3ccccc23)c([2H])c([2H])c1-c1c([2H])c([2H])c2c([2H])c([2H])c([2H])c([2H])c2c1[2H] ; [2H]c1c([2H])c(-c2c3ccccc3c(-c3ccc4oc5ccccc5c4c3)c3ccccc23)c([2H])c([2H])c1-c1c([2H])c([2H])c2c([2H])c([2H])c([2H])c([2H])c2c1[2H] ; [2H]c1c([2H])c(-c2c3ccccc3c(-c3c([2H])c([2H])c4c(oc5c([2H])c([2H])c([2H])c([2H])c54)c3[2H])c3ccccc23)c([2H])c([2H])c1-c1c([2H])c([2H])c2c([2H])c([2H])c([2H])c([2H])c2c1[2H]]

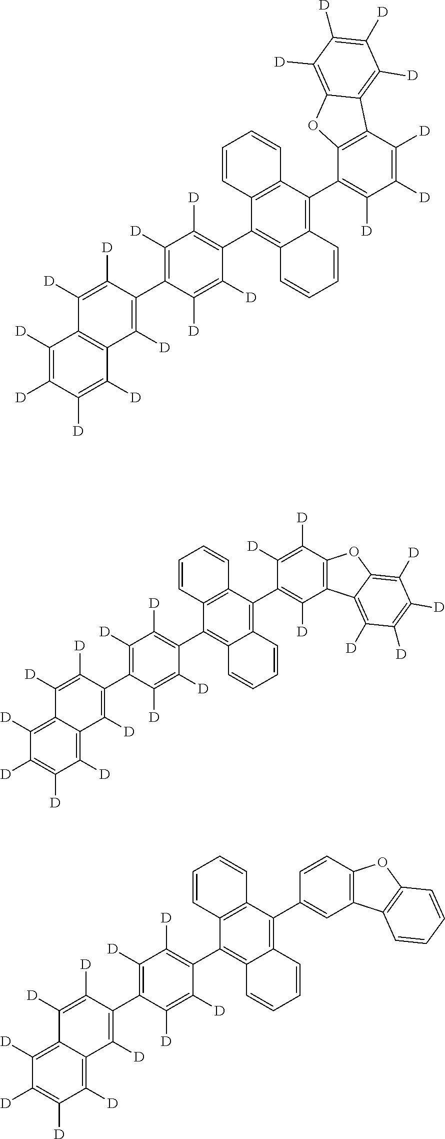 [SMILES: [2H]c1c([2H])c(-c2c3ccccc3c(-c3c([2H])c([2H])c([2H])c4c3oc3c([2H])c([2H])c([2H])c([2H])c34)c3ccccc23)c([2H])c([2H])c1-c1c([2H])c([2H])c2c([2H])c([2H])c([2H])c([2H])c2c1[2H] ; [2H]c1c([2H])c(-c2c3ccccc3c(-c3c([2H])c([2H])c4oc5c([2H])c([2H])c([2H])c([2H])c5c4c3[2H])c3ccccc23)c([2H])c([2H])c1-c1c([2H])c([2H])c2c([2H])c([2H])c([2H])c([2H])c2c1[2H] ; [2H]c1c([2H])c(-c2c3ccccc3c(-c3ccc4oc5ccccc5c4c3)c3ccccc23)c([2H])c([2H])c1-c1c([2H])c([2H])c2c([2H])c([2H])c([2H])c([2H])c2c1[2H]]

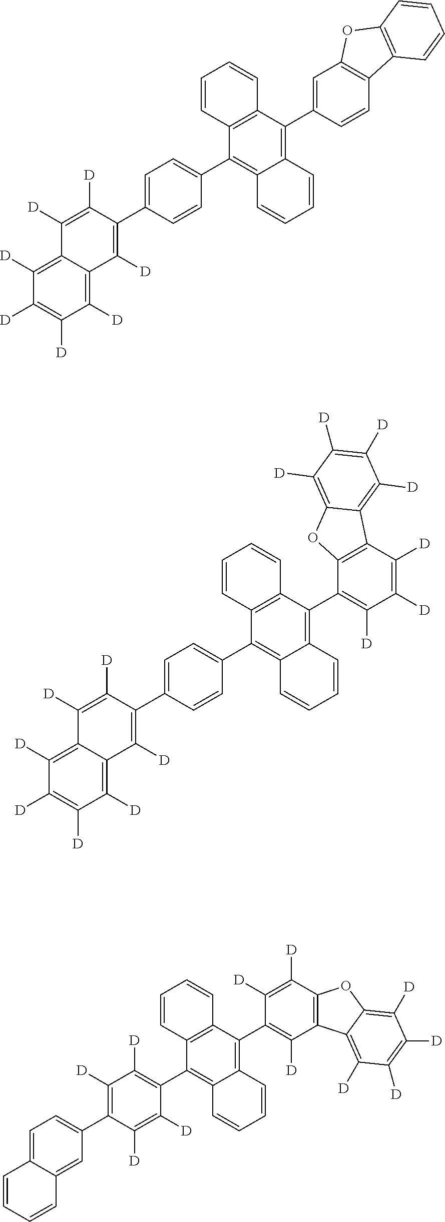 [SMILES: [2H]c1c([2H])c([2H])c2c([2H])c(-c3ccc(-c4c5ccccc5c(-c5ccc6c(c5)oc5ccccc56)c5ccccc45)cc3)c([2H])c([2H])c2c1[2H] ; [2H]c1c([2H])c([2H])c2c(oc3c(-c4c5ccccc5c(-c5ccc(-c6c([2H])c([2H])c7c([2H])c([2H])c([2H])c([2H])c7c6[2H])cc5)c5ccccc45)c([2H])c([2H])c([2H])c32)c1[2H] ; [2H]c1c([2H])c(-c2c3ccccc3c(-c3c([2H])c([2H])c4oc5c([2H])c([2H])c([2H])c([2H])c5c4c3[2H])c3ccccc23)c([2H])c([2H])c1-c1ccc2ccccc2c1]

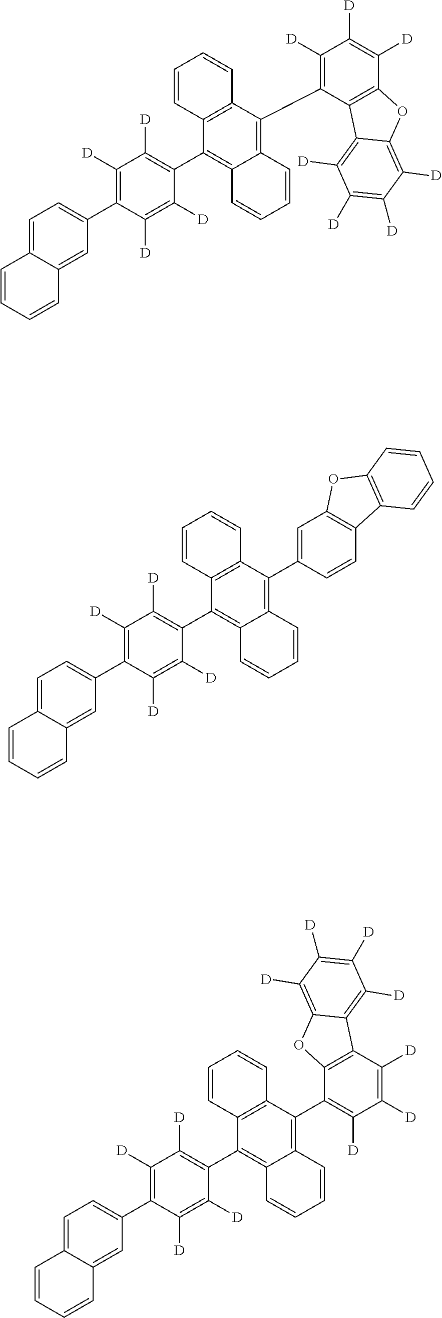 [SMILES: [2H]c1c([2H])c(-c2c3ccccc3c(-c3c([2H])c([2H])c([2H])c4oc5c([2H])c([2H])c([2H])c([2H])c5c34)c3ccccc23)c([2H])c([2H])c1-c1ccc2ccccc2c1 ; [2H]c1c([2H])c(-c2c3ccccc3c(-c3ccc4c(c3)oc3ccccc34)c3ccccc23)c([2H])c([2H])c1-c1ccc2ccccc2c1 ; [2H]c1c([2H])c(-c2c3ccccc3c(-c3c([2H])c([2H])c([2H])c4c3oc3c([2H])c([2H])c([2H])c([2H])c34)c3ccccc23)c([2H])c([2H])c1-c1ccc2ccccc2c1]

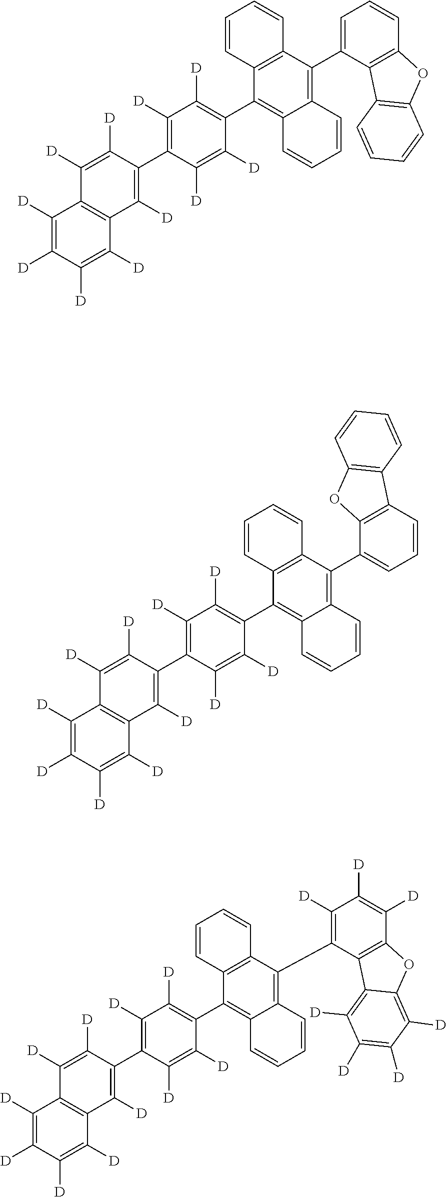 [SMILES: [2H]c1c([2H])c(-c2c3ccccc3c(-c3cccc4oc5ccccc5c34)c3ccccc23)c([2H])c([2H])c1-c1c([2H])c([2H])c2c([2H])c([2H])c([2H])c([2H])c2c1[2H] ; [2H]c1c([2H])c(-c2c3ccccc3c(-c3cccc4c3oc3ccccc34)c3ccccc23)c([2H])c([2H])c1-c1c([2H])c([2H])c2c([2H])c([2H])c([2H])c([2H])c2c1[2H] ; [2H]c1c([2H])c(-c2c3ccccc3c(-c3c([2H])c([2H])c([2H])c4oc5c([2H])c([2H])c([2H])c([2H])c5c34)c3ccccc23)c([2H])c([2H])c1-c1c([2H])c([2H])c2c([2H])c([2H])c([2H])c([2H])c2c1[2H]]

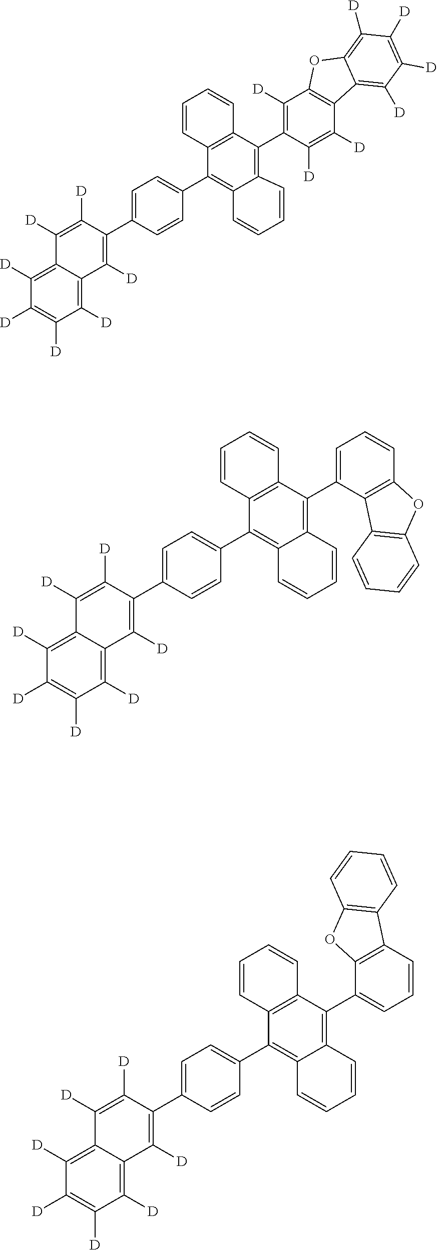 [SMILES: [2H]c1c([2H])c([2H])c2c(oc3c([2H])c(-c4c5ccccc5c(-c5ccc(-c6c([2H])c([2H])c7c([2H])c([2H])c([2H])c([2H])c7c6[2H])cc5)c5ccccc45)c([2H])c([2H])c32)c1[2H] ; [2H]c1c([2H])c([2H])c2c([2H])c(-c3ccc(-c4c5ccccc5c(-c5cccc6oc7ccccc7c56)c5ccccc45)cc3)c([2H])c([2H])c2c1[2H] ; [2H]c1c([2H])c([2H])c2c([2H])c(-c3ccc(-c4c5ccccc5c(-c5cccc6c5oc5ccccc56)c5ccccc45)cc3)c([2H])c([2H])c2c1[2H]]

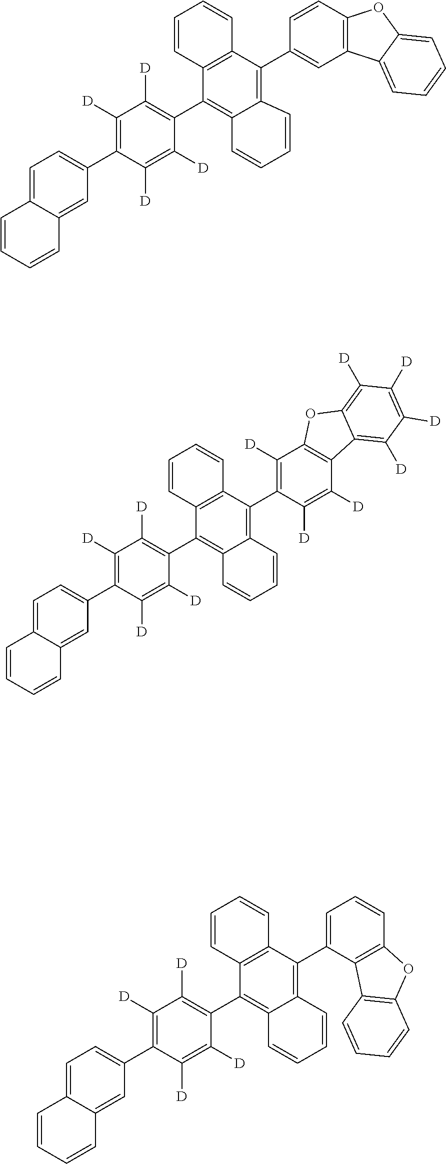 [SMILES: [2H]c1c([2H])c(-c2c3ccccc3c(-c3ccc4oc5ccccc5c4c3)c3ccccc23)c([2H])c([2H])c1-c1ccc2ccccc2c1 ; [2H]c1c([2H])c(-c2c3ccccc3c(-c3c([2H])c([2H])c4c(oc5c([2H])c([2H])c([2H])c([2H])c54)c3[2H])c3ccccc23)c([2H])c([2H])c1-c1ccc2ccccc2c1 ; [2H]c1c([2H])c(-c2c3ccccc3c(-c3cccc4oc5ccccc5c34)c3ccccc23)c([2H])c([2H])c1-c1ccc2ccccc2c1]

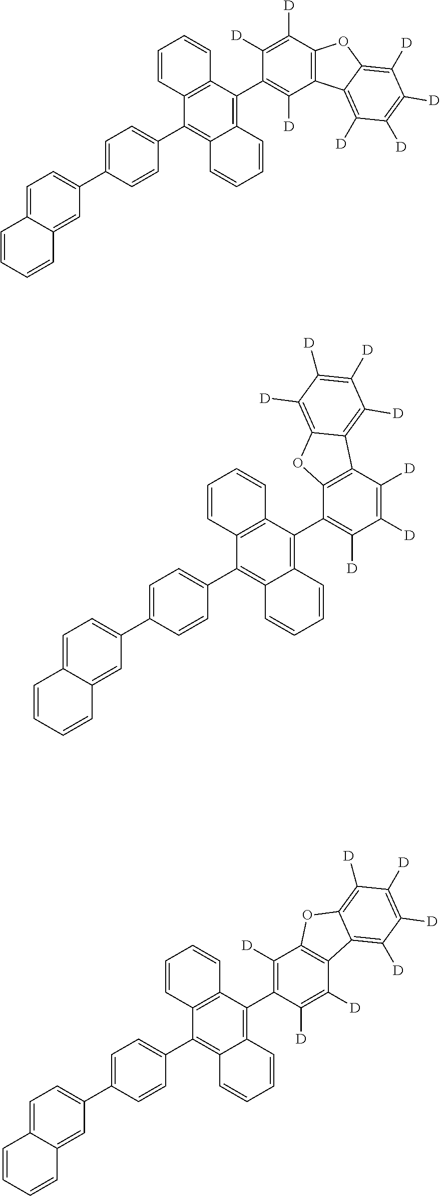 [SMILES: [2H]c1c([2H])c([2H])c2c(oc3c([2H])c([2H])c(-c4c5ccccc5c(-c5ccc(-c6ccc7ccccc7c6)cc5)c5ccccc45)c([2H])c32)c1[2H] ; [2H]c1c([2H])c([2H])c2c(oc3c(-c4c5ccccc5c(-c5ccc(-c6ccc7ccccc7c6)cc5)c5ccccc45)c([2H])c([2H])c([2H])c32)c1[2H] ; [2H]c1c([2H])c([2H])c2c(oc3c([2H])c(-c4c5ccccc5c(-c5ccc(-c6ccc7ccccc7c6)cc5)c5ccccc45)c([2H])c([2H])c32)c1[2H]]

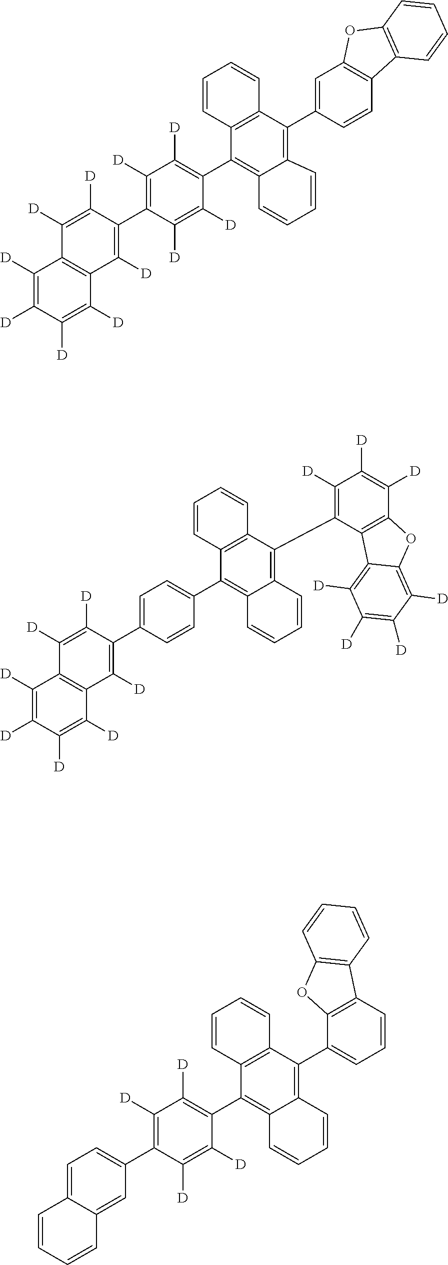 [SMILES: [2H]c1c([2H])c(-c2c3ccccc3c(-c3ccc4c(c3)oc3ccccc34)c3ccccc23)c([2H])c([2H])c1-c1c([2H])c([2H])c2c([2H])c([2H])c([2H])c([2H])c2c1[2H] ; [2H]c1c([2H])c([2H])c2c(oc3c([2H])c([2H])c([2H])c(-c4c5ccccc5c(-c5ccc(-c6c([2H])c([2H])c7c([2H])c([2H])c([2H])c([2H])c7c6[2H])cc5)c5ccccc45)c32)c1[2H] ; [2H]c1c([2H])c(-c2c3ccccc3c(-c3cccc4c3oc3ccccc34)c3ccccc23)c([2H])c([2H])c1-c1ccc2ccccc2c1]

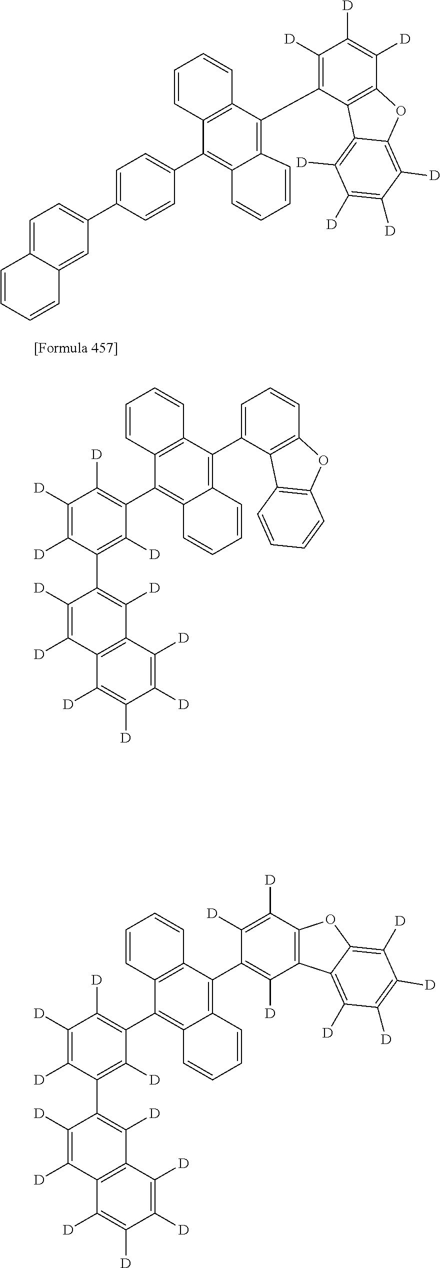 [SMILES: [2H]c1c([2H])c([2H])c2c(oc3c([2H])c([2H])c([2H])c(-c4c5ccccc5c(-c5ccc(-c6ccc7ccccc7c6)cc5)c5ccccc45)c32)c1[2H] ; [2H]c1c([2H])c(-c2c([2H])c([2H])c3c([2H])c([2H])c([2H])c([2H])c3c2[2H])c([2H])c(-c2c3ccccc3c(-c3cccc4oc5ccccc5c34)c3ccccc23)c1[2H] ; [2H]c1c([2H])c(-c2c([2H])c([2H])c3c([2H])c([2H])c([2H])c([2H])c3c2[2H])c([2H])c(-c2c3ccccc3c(-c3c([2H])c([2H])c4oc5c([2H])c([2H])c([2H])c([2H])c5c4c3[2H])c3ccccc23)c1[2H]]

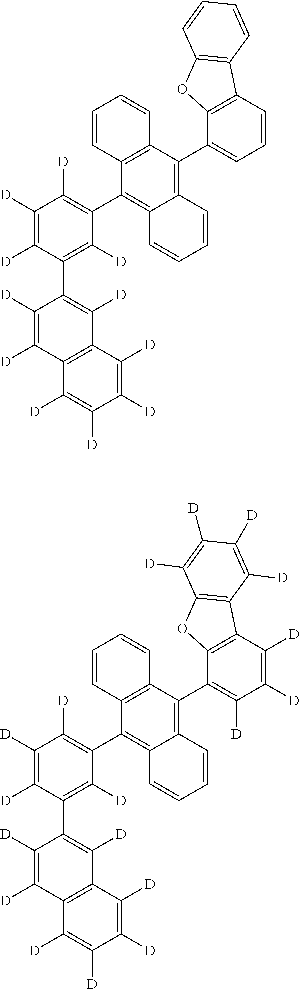 [SMILES: [2H]c1c([2H])c(-c2c([2H])c([2H])c3c([2H])c([2H])c([2H])c([2H])c3c2[2H])c([2H])c(-c2c3ccccc3c(-c3cccc4c3oc3ccccc34)c3ccccc23)c1[2H] ; [2H]c1c([2H])c(-c2c([2H])c([2H])c3c([2H])c([2H])c([2H])c([2H])c3c2[2H])c([2H])c(-c2c3ccccc3c(-c3c([2H])c([2H])c([2H])c4c3oc3c([2H])c([2H])c([2H])c([2H])c34)c3ccccc23)c1[2H]]

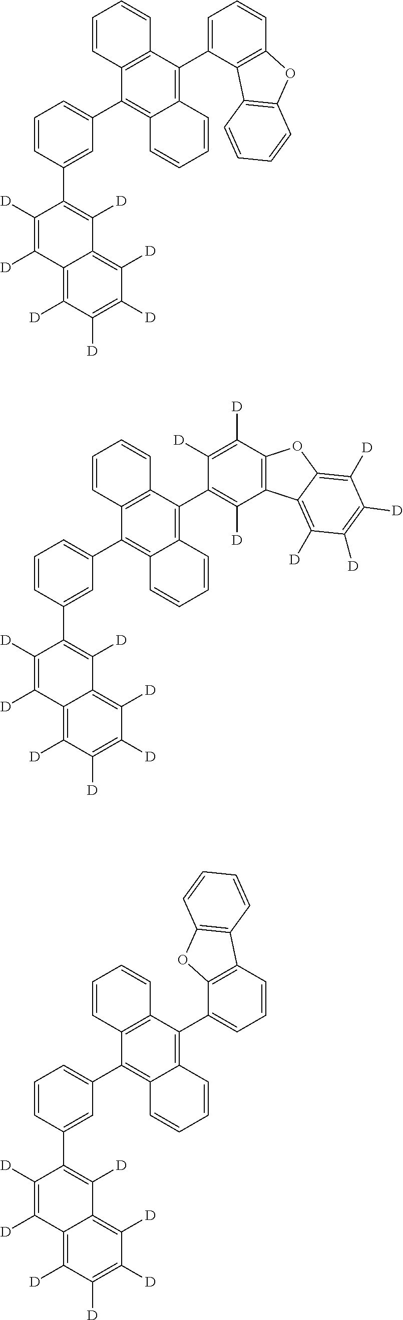 [SMILES: [2H]c1c([2H])c([2H])c2c([2H])c(-c3cccc(-c4c5ccccc5c(-c5cccc6oc7ccccc7c56)c5ccccc45)c3)c([2H])c([2H])c2c1[2H] ; [2H]c1c([2H])c([2H])c2c(oc3c([2H])c([2H])c(-c4c5ccccc5c(-c5cccc(-c6c([2H])c([2H])c7c([2H])c([2H])c([2H])c([2H])c7c6[2H])c5)c5ccccc45)c([2H])c32)c1[2H] ; [2H]c1c([2H])c([2H])c2c([2H])c(-c3cccc(-c4c5ccccc5c(-c5cccc6c5oc5ccccc56)c5ccccc45)c3)c([2H])c([2H])c2c1[2H]]

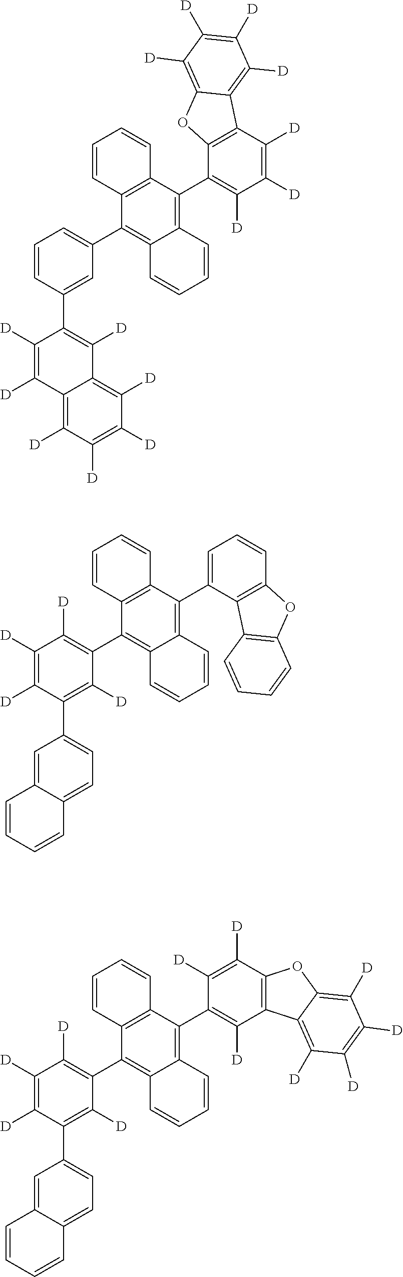 [SMILES: [2H]c1c([2H])c([2H])c2c(oc3c(-c4c5ccccc5c(-c5cccc(-c6c([2H])c([2H])c7c([2H])c([2H])c([2H])c([2H])c7c6[2H])c5)c5ccccc45)c([2H])c([2H])c([2H])c32)c1[2H] ; [2H]c1c([2H])c(-c2ccc3ccccc3c2)c([2H])c(-c2c3ccccc3c(-c3cccc4oc5ccccc5c34)c3ccccc23)c1[2H] ; [2H]c1c([2H])c(-c2ccc3ccccc3c2)c([2H])c(-c2c3ccccc3c(-c3c([2H])c([2H])c4oc5c([2H])c([2H])c([2H])c([2H])c5c4c3[2H])c3ccccc23)c1[2H]]

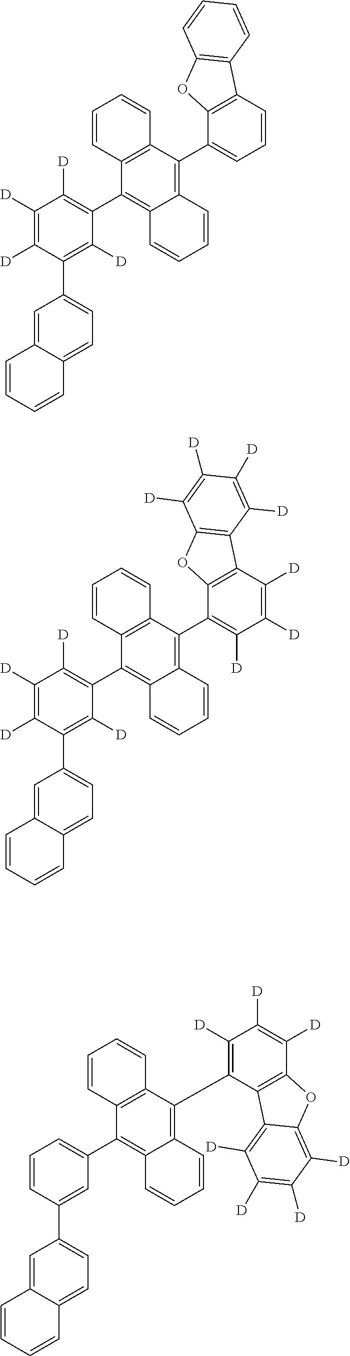 [SMILES: [2H]c1c([2H])c(-c2ccc3ccccc3c2)c([2H])c(-c2c3ccccc3c(-c3cccc4c3oc3ccccc34)c3ccccc23)c1[2H] ; [2H]c1c([2H])c(-c2ccc3ccccc3c2)c([2H])c(-c2c3ccccc3c(-c3c([2H])c([2H])c([2H])c4c3oc3c([2H])c([2H])c([2H])c([2H])c34)c3ccccc23)c1[2H] ; [2H]c1c([2H])c([2H])c2c(oc3c([2H])c([2H])c([2H])c(-c4c5ccccc5c(-c5cccc(-c6ccc7ccccc7c6)c5)c5ccccc45)c32)c1[2H]]

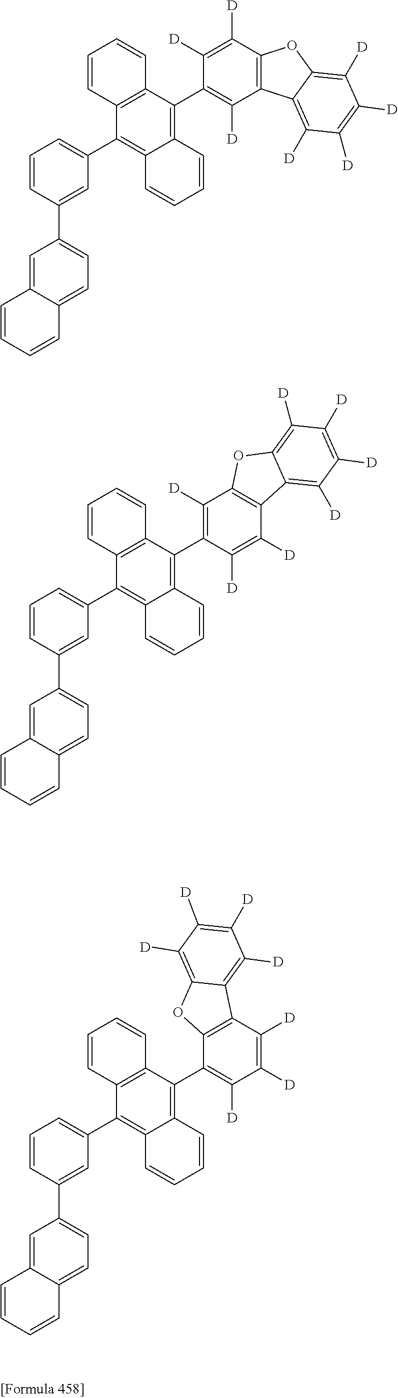 [SMILES: [2H]c1c([2H])c([2H])c2c(oc3c([2H])c([2H])c(-c4c5ccccc5c(-c5cccc(-c6ccc7ccccc7c6)c5)c5ccccc45)c([2H])c32)c1[2H] ; [2H]c1c([2H])c([2H])c2c(oc3c([2H])c(-c4c5ccccc5c(-c5cccc(-c6ccc7ccccc7c6)c5)c5ccccc45)c([2H])c([2H])c32)c1[2H] ; [2H]c1c([2H])c([2H])c2c(oc3c(-c4c5ccccc5c(-c5cccc(-c6ccc7ccccc7c6)c5)c5ccccc45)c([2H])c([2H])c([2H])c32)c1[2H]]

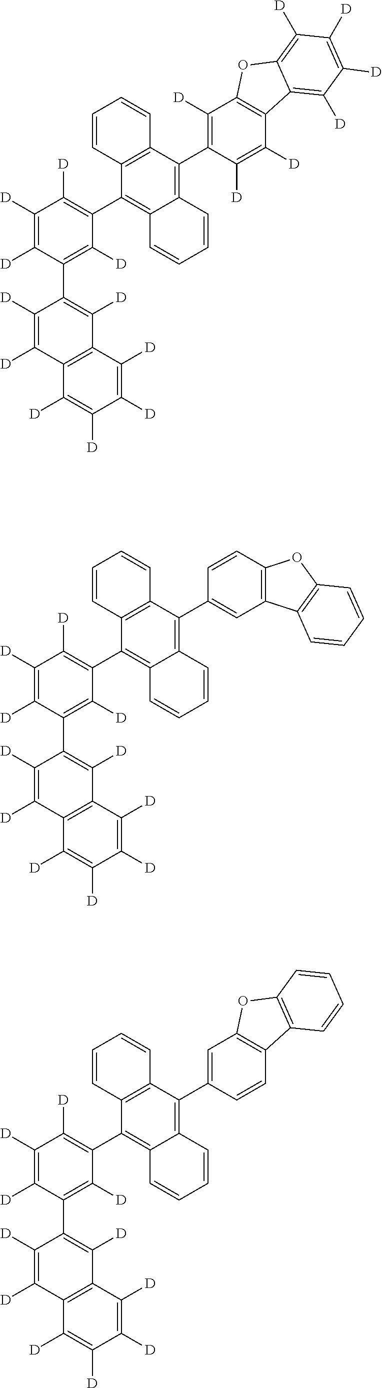 [SMILES: [2H]c1c([2H])c(-c2c([2H])c([2H])c3c([2H])c([2H])c([2H])c([2H])c3c2[2H])c([2H])c(-c2c3ccccc3c(-c3c([2H])c([2H])c4c(oc5c([2H])c([2H])c([2H])c([2H])c54)c3[2H])c3ccccc23)c1[2H] ; [2H]c1c([2H])c(-c2c([2H])c([2H])c3c([2H])c([2H])c([2H])c([2H])c3c2[2H])c([2H])c(-c2c3ccccc3c(-c3ccc4oc5ccccc5c4c3)c3ccccc23)c1[2H] ; [2H]c1c([2H])c(-c2c([2H])c([2H])c3c([2H])c([2H])c([2H])c([2H])c3c2[2H])c([2H])c(-c2c3ccccc3c(-c3ccc4c(c3)oc3ccccc34)c3ccccc23)c1[2H]]

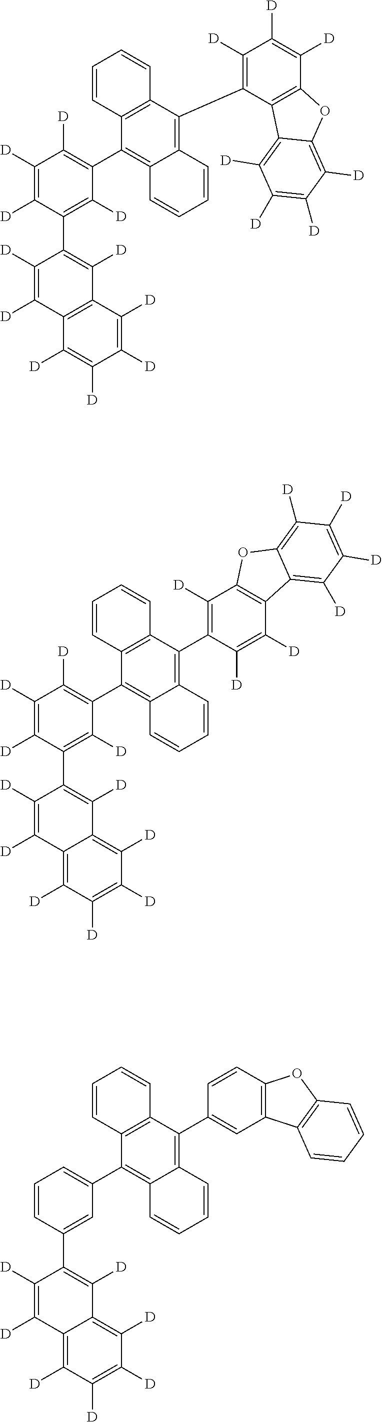 [SMILES: [2H]c1c([2H])c(-c2c([2H])c([2H])c3c([2H])c([2H])c([2H])c([2H])c3c2[2H])c([2H])c(-c2c3ccccc3c(-c3c([2H])c([2H])c([2H])c4oc5c([2H])c([2H])c([2H])c([2H])c5c34)c3ccccc23)c1[2H] ; [2H]c1c([2H])c(-c2c([2H])c([2H])c3c([2H])c([2H])c([2H])c([2H])c3c2[2H])c([2H])c(-c2c3ccccc3c(-c3c([2H])c([2H])c4c(oc5c([2H])c([2H])c([2H])c([2H])c54)c3[2H])c3ccccc23)c1[2H] ; [2H]c1c([2H])c([2H])c2c([2H])c(-c3cccc(-c4c5ccccc5c(-c5ccc6oc7ccccc7c6c5)c5ccccc45)c3)c([2H])c([2H])c2c1[2H]]

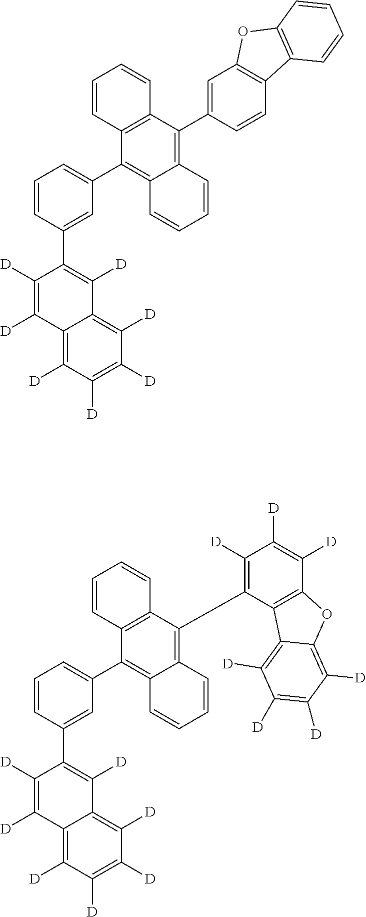 [SMILES: [2H]c1c([2H])c([2H])c2c([2H])c(-c3cccc(-c4c5ccccc5c(-c5ccc6c(c5)oc5ccccc56)c5ccccc45)c3)c([2H])c([2H])c2c1[2H] ; [2H]c1c([2H])c([2H])c2c(oc3c([2H])c([2H])c([2H])c(-c4c5ccccc5c(-c5cccc(-c6c([2H])c([2H])c7c([2H])c([2H])c([2H])c([2H])c7c6[2H])c5)c5ccccc45)c32)c1[2H]]

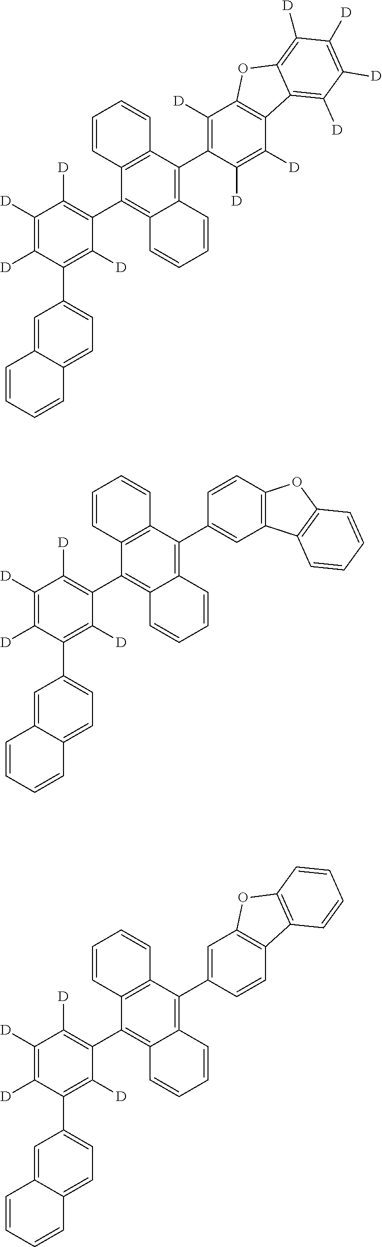 [SMILES: [2H]c1c([2H])c(-c2ccc3ccccc3c2)c([2H])c(-c2c3ccccc3c(-c3c([2H])c([2H])c4c(oc5c([2H])c([2H])c([2H])c([2H])c54)c3[2H])c3ccccc23)c1[2H] ; [2H]c1c([2H])c(-c2ccc3ccccc3c2)c([2H])c(-c2c3ccccc3c(-c3ccc4oc5ccccc5c4c3)c3ccccc23)c1[2H] ; [2H]c1c([2H])c(-c2ccc3ccccc3c2)c([2H])c(-c2c3ccccc3c(-c3ccc4c(c3)oc3ccccc34)c3ccccc23)c1[2H]]

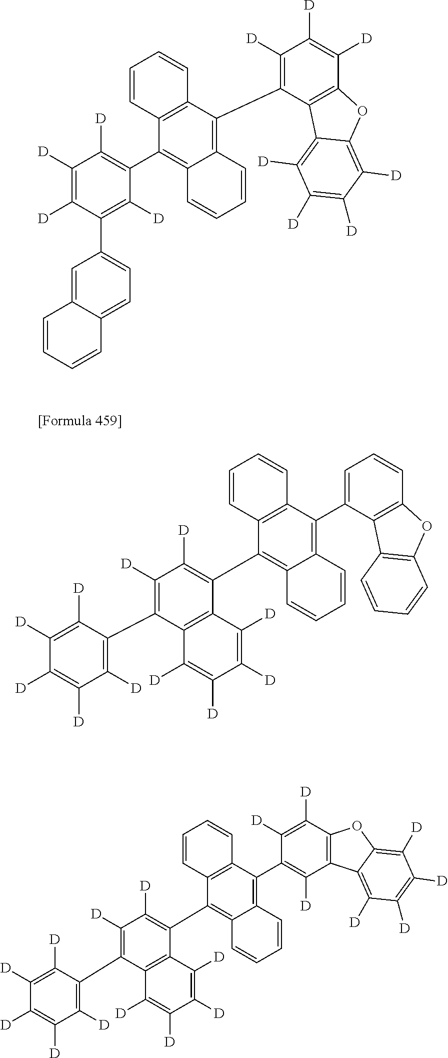 [SMILES: [2H]c1c([2H])c(-c2ccc3ccccc3c2)c([2H])c(-c2c3ccccc3c(-c3c([2H])c([2H])c([2H])c4oc5c([2H])c([2H])c([2H])c([2H])c5c34)c3ccccc23)c1[2H] ; [2H]c1c([2H])c([2H])c(-c2c([2H])c([2H])c(-c3c4ccccc4c(-c4cccc5oc6ccccc6c45)c4ccccc34)c3c([2H])c([2H])c([2H])c([2H])c23)c([2H])c1[2H] ; [2H]c1c([2H])c([2H])c(-c2c([2H])c([2H])c(-c3c4ccccc4c(-c4c([2H])c([2H])c5oc6c([2H])c([2H])c([2H])c([2H])c6c5c4[2H])c4ccccc34)c3c([2H])c([2H])c([2H])c([2H])c23)c([2H])c1[2H]]

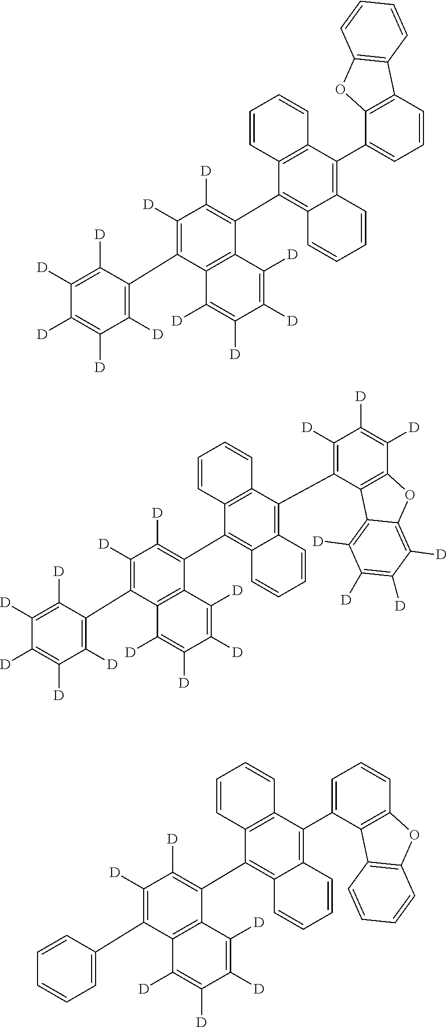 [SMILES: [2H]c1c([2H])c([2H])c(-c2c([2H])c([2H])c(-c3c4ccccc4c(-c4cccc5c4oc4ccccc45)c4ccccc34)c3c([2H])c([2H])c([2H])c([2H])c23)c([2H])c1[2H] ; [2H]c1c([2H])c([2H])c(-c2c([2H])c([2H])c(-c3c4ccccc4c(-c4c([2H])c([2H])c([2H])c5oc6c([2H])c([2H])c([2H])c([2H])c6c45)c4ccccc34)c3c([2H])c([2H])c([2H])c([2H])c23)c([2H])c1[2H] ; [2H]c1c([2H])c([2H])c2c(-c3c4ccccc4c(-c4cccc5oc6ccccc6c45)c4ccccc34)c([2H])c([2H])c(-c3ccccc3)c2c1[2H]]

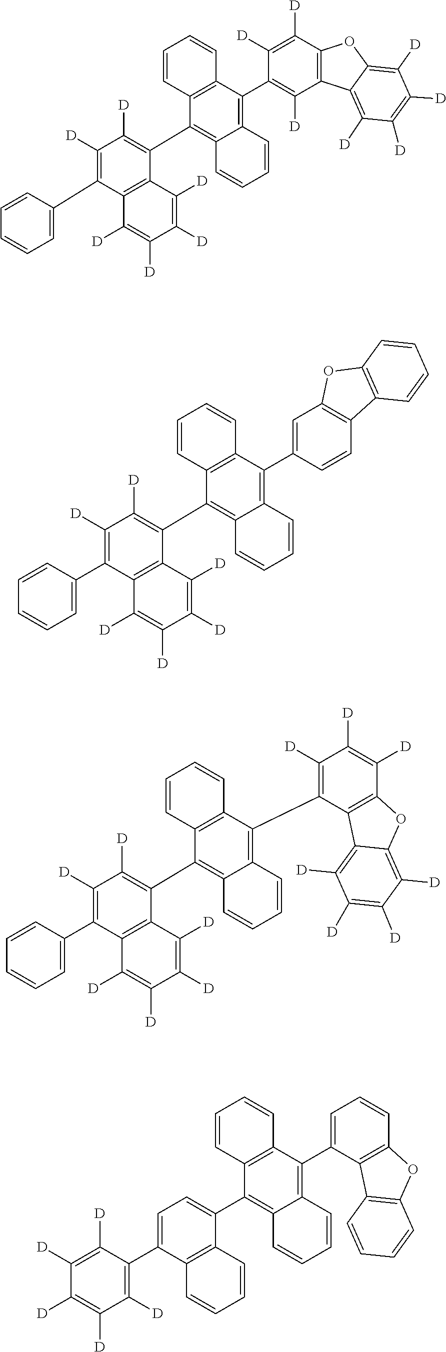

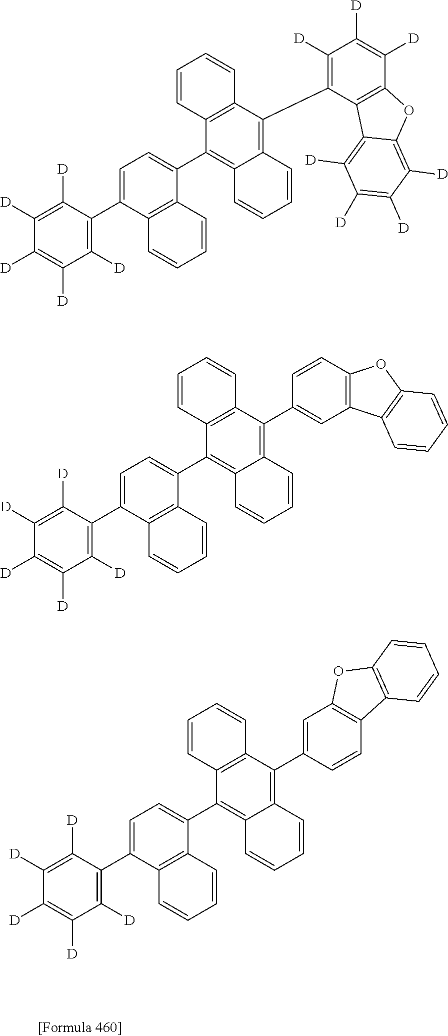 [SMILES: [2H]c1c([2H])c([2H])c(-c2ccc(-c3c4ccccc4c(-c4c([2H])c([2H])c([2H])c5oc6c([2H])c([2H])c([2H])c([2H])c6c45)c4ccccc34)c3ccccc23)c([2H])c1[2H] ; [2H]c1c([2H])c([2H])c(-c2ccc(-c3c4ccccc4c(-c4ccc5oc6ccccc6c5c4)c4ccccc34)c3ccccc23)c([2H])c1[2H] ; [2H]c1c([2H])c([2H])c(-c2ccc(-c3c4ccccc4c(-c4ccc5c(c4)oc4ccccc45)c4ccccc34)c3ccccc23)c([2H])c1[2H]]

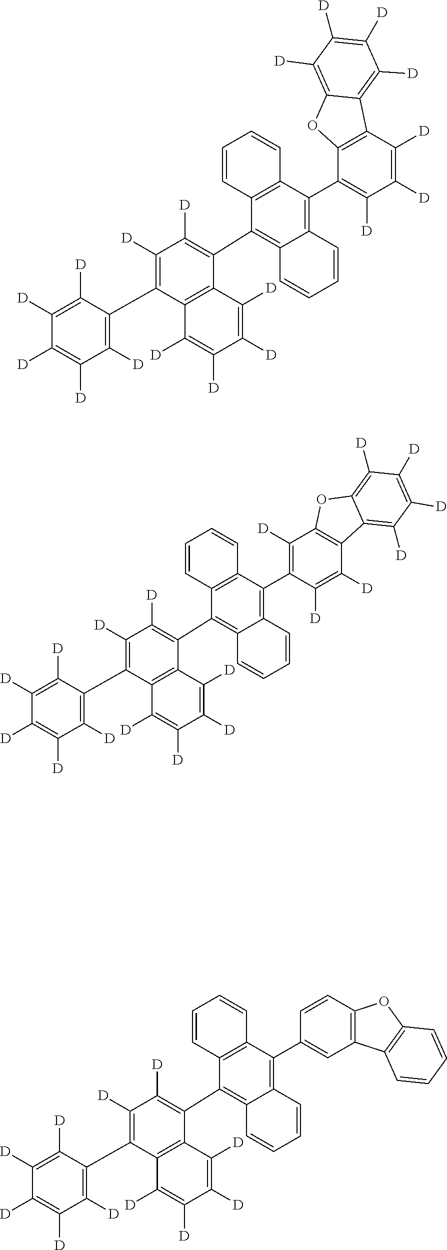 [SMILES: [2H]c1c([2H])c([2H])c(-c2c([2H])c([2H])c(-c3c4ccccc4c(-c4c([2H])c([2H])c([2H])c5c4oc4c([2H])c([2H])c([2H])c([2H])c45)c4ccccc34)c3c([2H])c([2H])c([2H])c([2H])c23)c([2H])c1[2H] ; [2H]c1c([2H])c([2H])c(-c2c([2H])c([2H])c(-c3c4ccccc4c(-c4c([2H])c([2H])c5c(oc6c([2H])c([2H])c([2H])c([2H])c65)c4[2H])c4ccccc34)c3c([2H])c([2H])c([2H])c([2H])c23)c([2H])c1[2H] ; [2H]c1c([2H])c([2H])c(-c2c([2H])c([2H])c(-c3c4ccccc4c(-c4ccc5oc6ccccc6c5c4)c4ccccc34)c3c([2H])c([2H])c([2H])c([2H])c23)c([2H])c1[2H]]

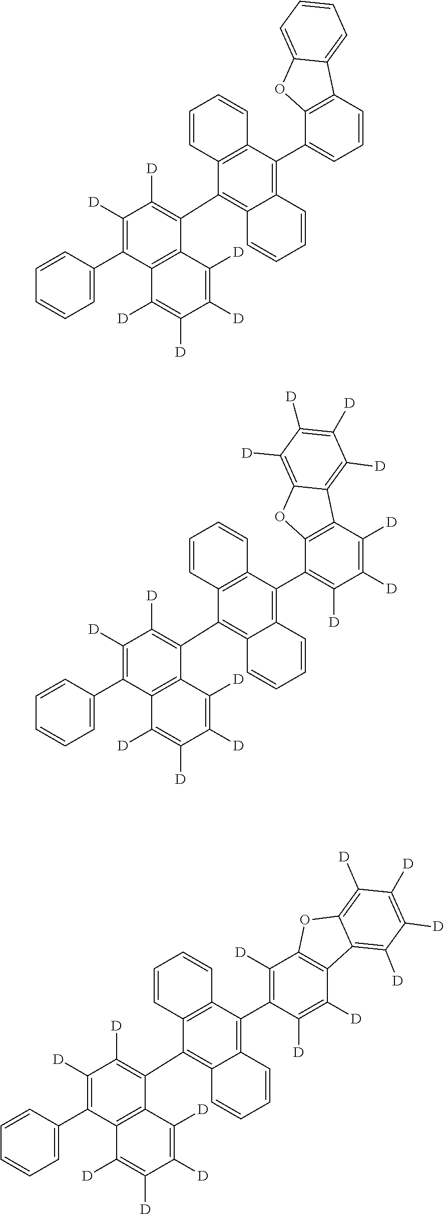 [SMILES: [2H]c1c([2H])c([2H])c2c(-c3c4ccccc4c(-c4cccc5c4oc4ccccc45)c4ccccc34)c([2H])c([2H])c(-c3ccccc3)c2c1[2H] ; [2H]c1c([2H])c([2H])c2c(oc3c(-c4c5ccccc5c(-c5c([2H])c([2H])c(-c6ccccc6)c6c([2H])c([2H])c([2H])c([2H])c56)c5ccccc45)c([2H])c([2H])c([2H])c32)c1[2H] ; [2H]c1c([2H])c([2H])c2c(oc3c([2H])c(-c4c5ccccc5c(-c5c([2H])c([2H])c(-c6ccccc6)c6c([2H])c([2H])c([2H])c([2H])c56)c5ccccc45)c([2H])c([2H])c32)c1[2H]]

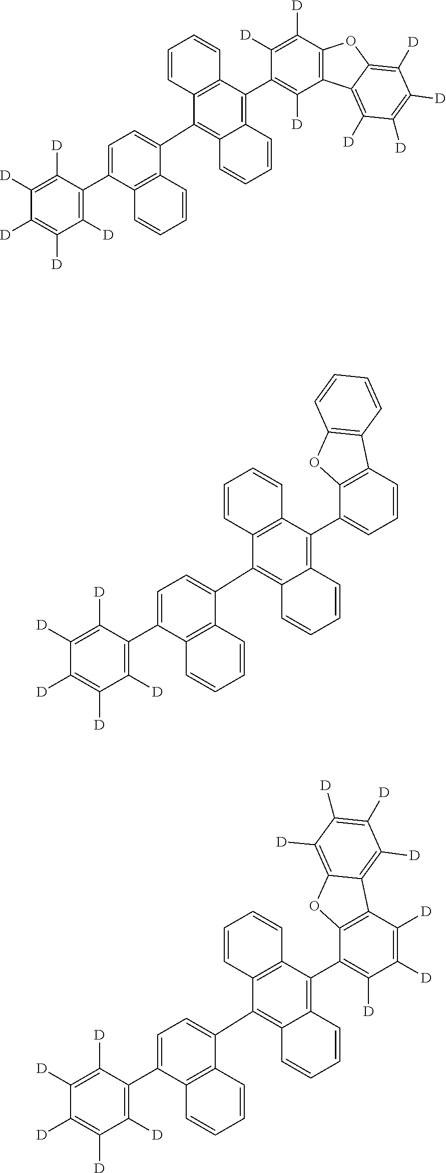 [SMILES: [2H]c1c([2H])c([2H])c(-c2ccc(-c3c4ccccc4c(-c4c([2H])c([2H])c5oc6c([2H])c([2H])c([2H])c([2H])c6c5c4[2H])c4ccccc34)c3ccccc23)c([2H])c1[2H] ; [2H]c1c([2H])c([2H])c(-c2ccc(-c3c4ccccc4c(-c4cccc5c4oc4ccccc45)c4ccccc34)c3ccccc23)c([2H])c1[2H] ; [2H]c1c([2H])c([2H])c(-c2ccc(-c3c4ccccc4c(-c4c([2H])c([2H])c([2H])c5c4oc4c([2H])c([2H])c([2H])c([2H])c45)c4ccccc34)c3ccccc23)c([2H])c1[2H]]

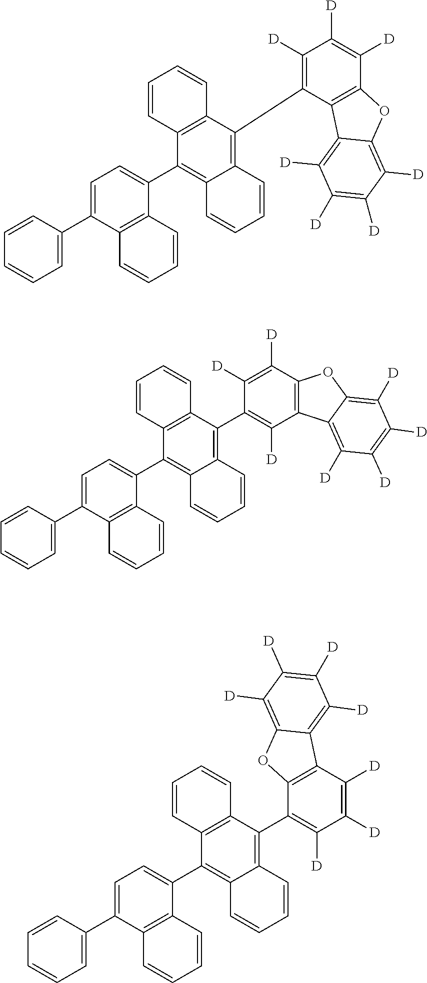 [SMILES: [2H]c1c([2H])c([2H])c2c(oc3c([2H])c([2H])c([2H])c(-c4c5ccccc5c(-c5ccc(-c6ccccc6)c6ccccc56)c5ccccc45)c32)c1[2H] ; [2H]c1c([2H])c([2H])c2c(oc3c([2H])c([2H])c(-c4c5ccccc5c(-c5ccc(-c6ccccc6)c6ccccc56)c5ccccc45)c([2H])c32)c1[2H] ; [2H]c1c([2H])c([2H])c2c(oc3c(-c4c5ccccc5c(-c5ccc(-c6ccccc6)c6ccccc56)c5ccccc45)c([2H])c([2H])c([2H])c32)c1[2H]]

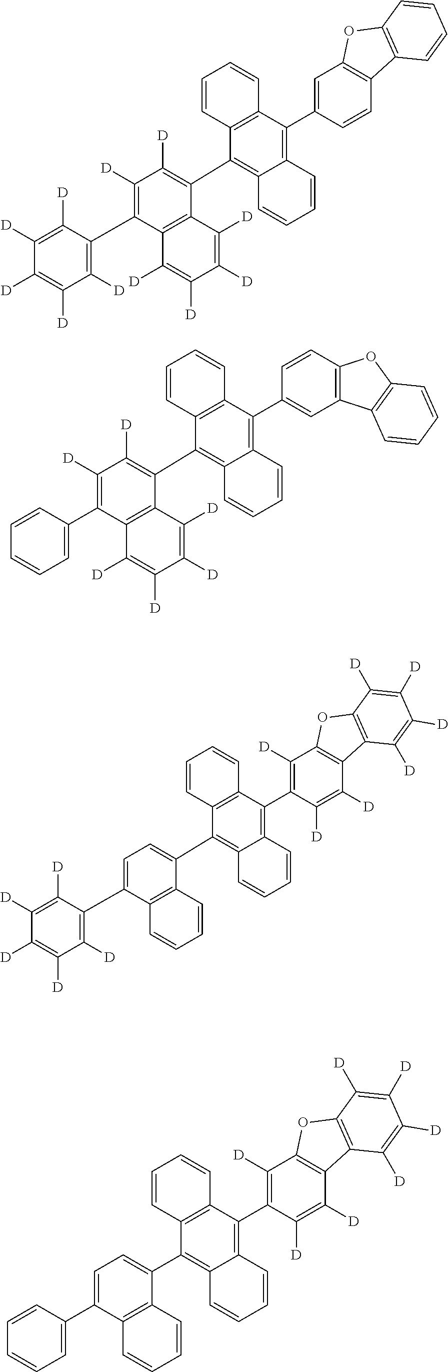

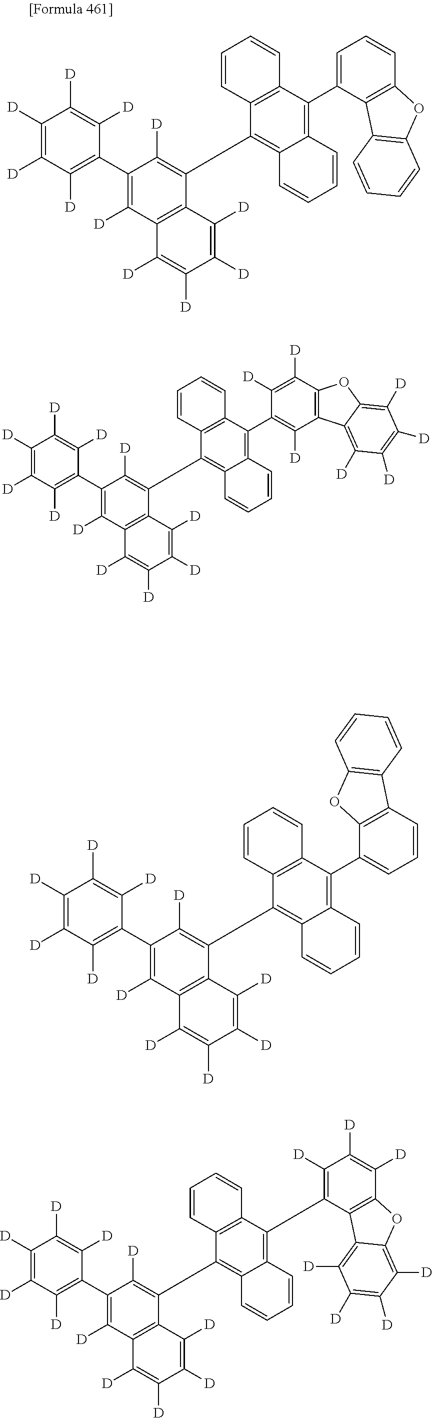

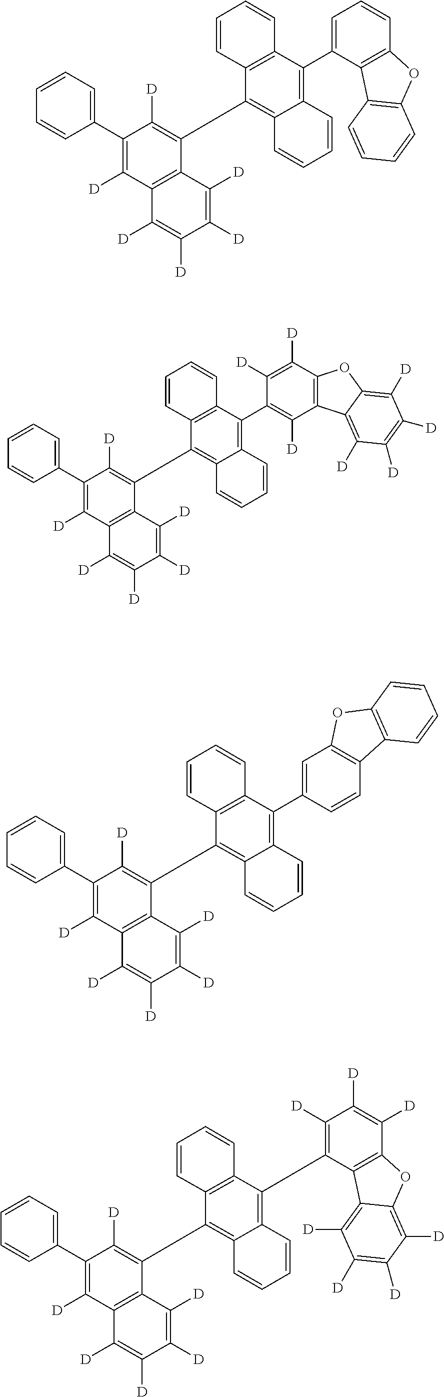

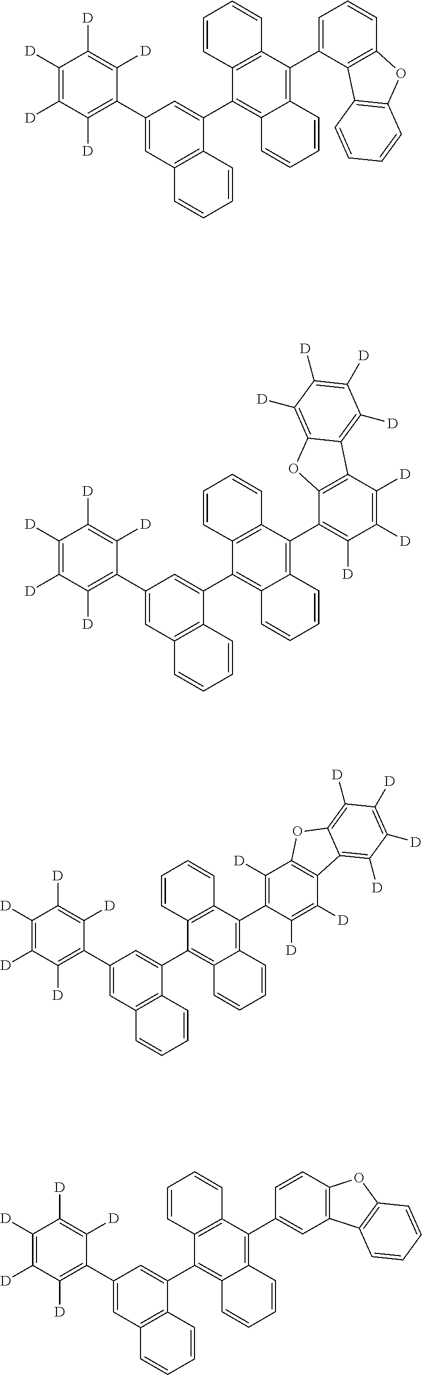 [SMILES: [2H]c1c([2H])c([2H])c(-c2cc(-c3c4ccccc4c(-c4cccc5oc6ccccc6c45)c4ccccc34)c3ccccc3c2)c([2H])c1[2H] ; [2H]c1c([2H])c([2H])c(-c2cc(-c3c4ccccc4c(-c4c([2H])c([2H])c([2H])c5c4oc4c([2H])c([2H])c([2H])c([2H])c45)c4ccccc34)c3ccccc3c2)c([2H])c1[2H] ; [2H]c1c([2H])c([2H])c(-c2cc(-c3c4ccccc4c(-c4c([2H])c([2H])c5c(oc6c([2H])c([2H])c([2H])c([2H])c65)c4[2H])c4ccccc34)c3ccccc3c2)c([2H])c1[2H] ; [2H]c1c([2H])c([2H])c(-c2cc(-c3c4ccccc4c(-c4ccc5oc6ccccc6c5c4)c4ccccc34)c3ccccc3c2)c([2H])c1[2H]]

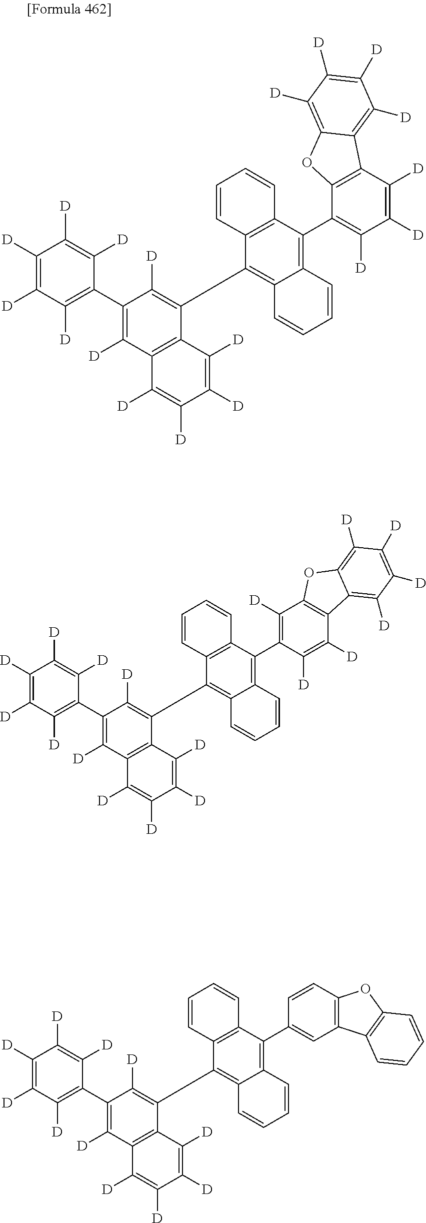 [SMILES: [2H]c1c([2H])c([2H])c(-c2c([2H])c(-c3c4ccccc4c(-c4c([2H])c([2H])c([2H])c5c4oc4c([2H])c([2H])c([2H])c([2H])c45)c4ccccc34)c3c([2H])c([2H])c([2H])c([2H])c3c2[2H])c([2H])c1[2H] ; [2H]c1c([2H])c([2H])c(-c2c([2H])c(-c3c4ccccc4c(-c4c([2H])c([2H])c5c(oc6c([2H])c([2H])c([2H])c([2H])c65)c4[2H])c4ccccc34)c3c([2H])c([2H])c([2H])c([2H])c3c2[2H])c([2H])c1[2H] ; [2H]c1c([2H])c([2H])c(-c2c([2H])c(-c3c4ccccc4c(-c4ccc5oc6ccccc6c5c4)c4ccccc34)c3c([2H])c([2H])c([2H])c([2H])c3c2[2H])c([2H])c1[2H]]

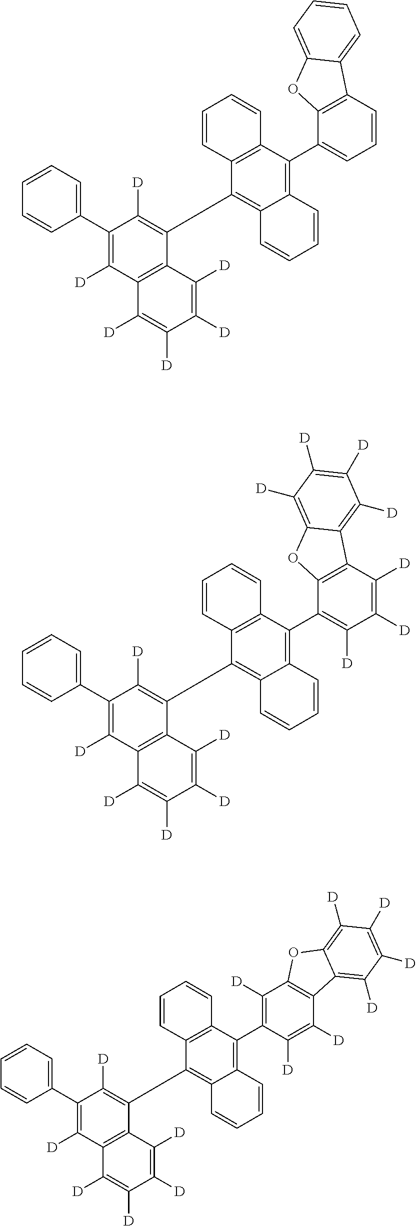 [SMILES: [2H]c1c([2H])c([2H])c2c(-c3c4ccccc4c(-c4cccc5c4oc4ccccc45)c4ccccc34)c([2H])c(-c3ccccc3)c([2H])c2c1[2H] ; [2H]c1c([2H])c([2H])c2c(oc3c(-c4c5ccccc5c(-c5c([2H])c(-c6ccccc6)c([2H])c6c([2H])c([2H])c([2H])c([2H])c56)c5ccccc45)c([2H])c([2H])c([2H])c32)c1[2H] ; [2H]c1c([2H])c([2H])c2c(oc3c([2H])c(-c4c5ccccc5c(-c5c([2H])c(-c6ccccc6)c([2H])c6c([2H])c([2H])c([2H])c([2H])c56)c5ccccc45)c([2H])c([2H])c32)c1[2H]]

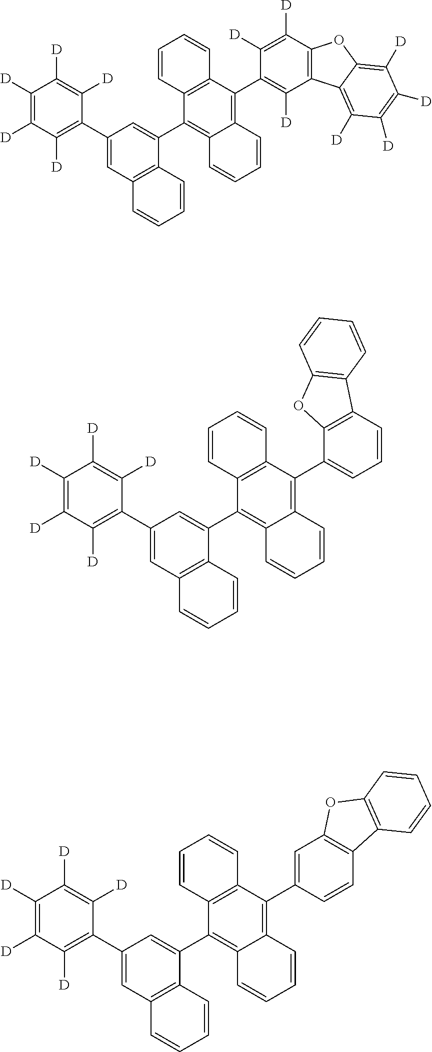 [SMILES: [2H]c1c([2H])c([2H])c(-c2cc(-c3c4ccccc4c(-c4c([2H])c([2H])c5oc6c([2H])c([2H])c([2H])c([2H])c6c5c4[2H])c4ccccc34)c3ccccc3c2)c([2H])c1[2H] ; [2H]c1c([2H])c([2H])c(-c2cc(-c3c4ccccc4c(-c4cccc5c4oc4ccccc45)c4ccccc34)c3ccccc3c2)c([2H])c1[2H] ; [2H]c1c([2H])c([2H])c(-c2cc(-c3c4ccccc4c(-c4ccc5c(c4)oc4ccccc45)c4ccccc34)c3ccccc3c2)c([2H])c1[2H]]

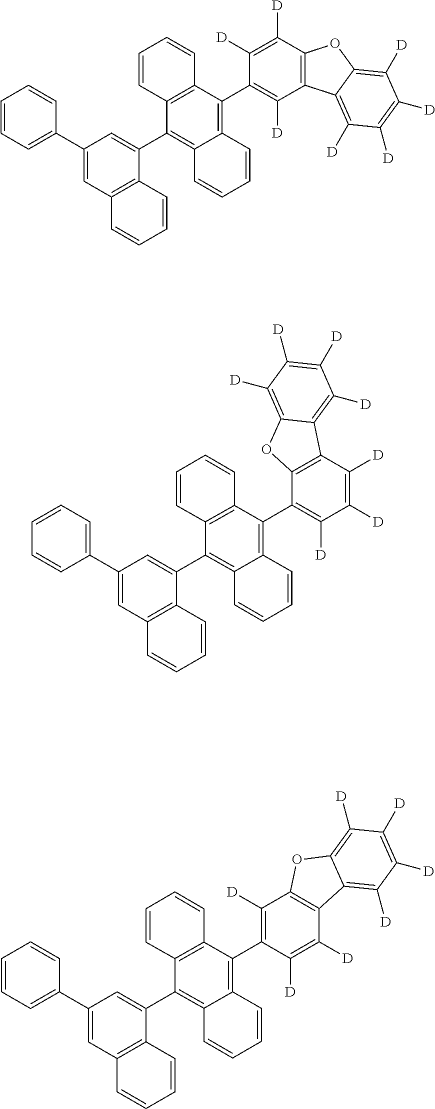 [SMILES: [2H]c1c([2H])c([2H])c2c(oc3c([2H])c([2H])c(-c4c5ccccc5c(-c5cc(-c6ccccc6)cc6ccccc56)c5ccccc45)c([2H])c32)c1[2H] ; [2H]c1c([2H])c([2H])c2c(oc3c(-c4c5ccccc5c(-c5cc(-c6ccccc6)cc6ccccc56)c5ccccc45)c([2H])c([2H])c([2H])c32)c1[2H] ; [2H]c1c([2H])c([2H])c2c(oc3c([2H])c(-c4c5ccccc5c(-c5cc(-c6ccccc6)cc6ccccc56)c5ccccc45)c([2H])c([2H])c32)c1[2H]]

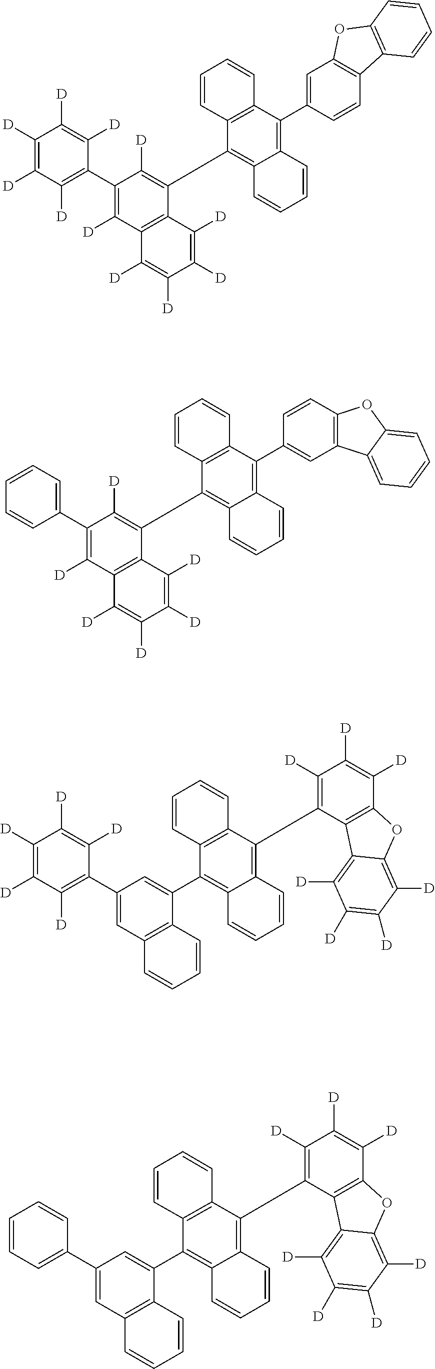 [SMILES: [2H]c1c([2H])c([2H])c(-c2c([2H])c(-c3c4ccccc4c(-c4ccc5c(c4)oc4ccccc45)c4ccccc34)c3c([2H])c([2H])c([2H])c([2H])c3c2[2H])c([2H])c1[2H] ; [2H]c1c([2H])c([2H])c2c(-c3c4ccccc4c(-c4ccc5oc6ccccc6c5c4)c4ccccc34)c([2H])c(-c3ccccc3)c([2H])c2c1[2H] ; [2H]c1c([2H])c([2H])c(-c2cc(-c3c4ccccc4c(-c4c([2H])c([2H])c([2H])c5oc6c([2H])c([2H])c([2H])c([2H])c6c45)c4ccccc34)c3ccccc3c2)c([2H])c1[2H] ; [2H]c1c([2H])c([2H])c2c(oc3c([2H])c([2H])c([2H])c(-c4c5ccccc5c(-c5cc(-c6ccccc6)cc6ccccc56)c5ccccc45)c32)c1[2H]]

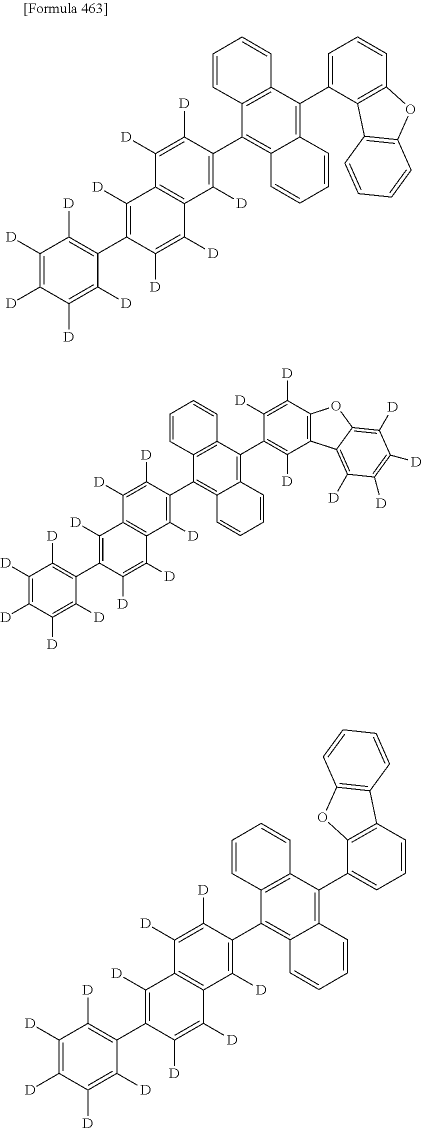 [SMILES: [2H]c1c([2H])c([2H])c(-c2c([2H])c([2H])c3c([2H])c(-c4c5ccccc5c(-c5cccc6oc7ccccc7c56)c5ccccc45)c([2H])c([2H])c3c2[2H])c([2H])c1[2H] ; [2H]c1c([2H])c([2H])c(-c2c([2H])c([2H])c3c([2H])c(-c4c5ccccc5c(-c5c([2H])c([2H])c6oc7c([2H])c([2H])c([2H])c([2H])c7c6c5[2H])c5ccccc45)c([2H])c([2H])c3c2[2H])c([2H])c1[2H] ; [2H]c1c([2H])c([2H])c(-c2c([2H])c([2H])c3c([2H])c(-c4c5ccccc5c(-c5cccc6c5oc5ccccc56)c5ccccc45)c([2H])c([2H])c3c2[2H])c([2H])c1[2H]]

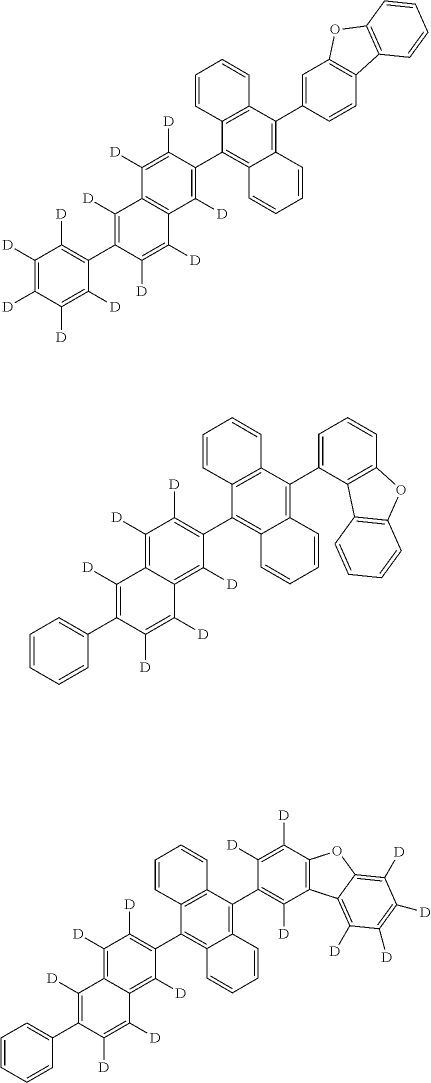 [SMILES: [2H]c1c([2H])c([2H])c(-c2c([2H])c([2H])c3c([2H])c(-c4c5ccccc5c(-c5ccc6c(c5)oc5ccccc56)c5ccccc45)c([2H])c([2H])c3c2[2H])c([2H])c1[2H] ; [2H]c1c(-c2c3ccccc3c(-c3cccc4oc5ccccc5c34)c3ccccc23)c([2H])c2c([2H])c([2H])c(-c3ccccc3)c([2H])c2c1[2H] ; [2H]c1c([2H])c([2H])c2c(oc3c([2H])c([2H])c(-c4c5ccccc5c(-c5c([2H])c([2H])c6c([2H])c(-c7ccccc7)c([2H])c([2H])c6c5[2H])c5ccccc45)c([2H])c32)c1[2H]]

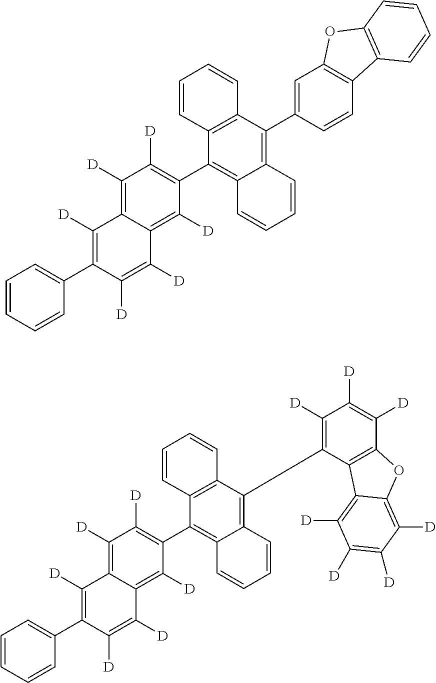 [SMILES: [2H]c1c(-c2c3ccccc3c(-c3ccc4c(c3)oc3ccccc34)c3ccccc23)c([2H])c2c([2H])c([2H])c(-c3ccccc3)c([2H])c2c1[2H] ; [2H]c1c([2H])c([2H])c2c(oc3c([2H])c([2H])c([2H])c(-c4c5ccccc5c(-c5c([2H])c([2H])c6c([2H])c(-c7ccccc7)c([2H])c([2H])c6c5[2H])c5ccccc45)c32)c1[2H]]

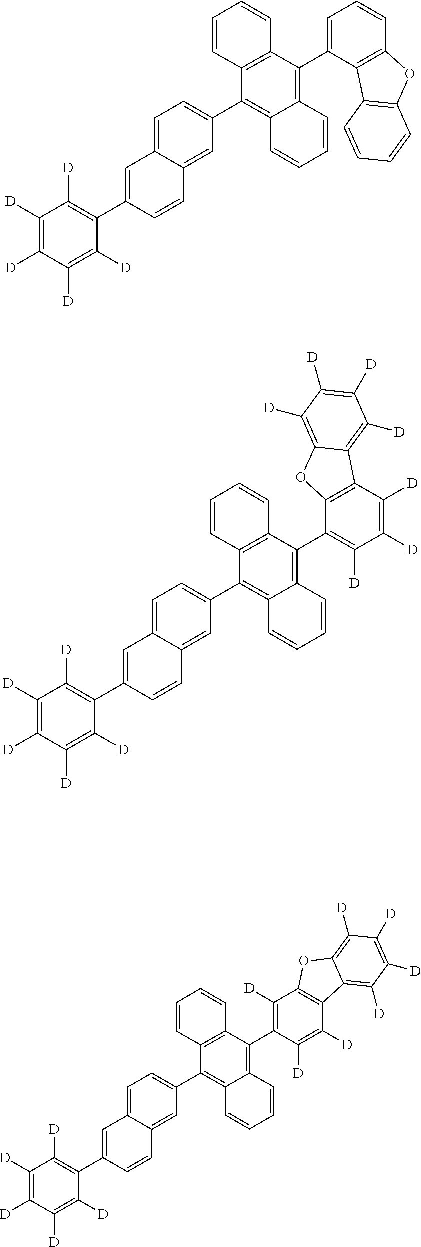 [SMILES: [2H]c1c([2H])c([2H])c(-c2ccc3cc(-c4c5ccccc5c(-c5cccc6oc7ccccc7c56)c5ccccc45)ccc3c2)c([2H])c1[2H] ; [2H]c1c([2H])c([2H])c(-c2ccc3cc(-c4c5ccccc5c(-c5c([2H])c([2H])c([2H])c6c5oc5c([2H])c([2H])c([2H])c([2H])c56)c5ccccc45)ccc3c2)c([2H])c1[2H] ; [2H]c1c([2H])c([2H])c(-c2ccc3cc(-c4c5ccccc5c(-c5c([2H])c([2H])c6c(oc7c([2H])c([2H])c([2H])c([2H])c76)c5[2H])c5ccccc45)ccc3c2)c([2H])c1[2H]]

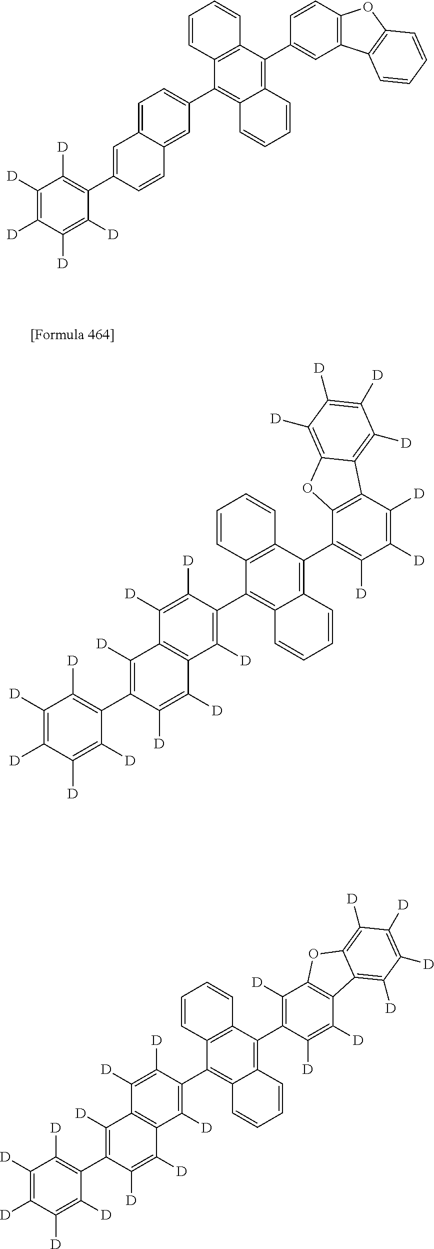 [SMILES: [2H]c1c([2H])c([2H])c(-c2ccc3cc(-c4c5ccccc5c(-c5ccc6oc7ccccc7c6c5)c5ccccc45)ccc3c2)c([2H])c1[2H] ; [2H]c1c([2H])c([2H])c(-c2c([2H])c([2H])c3c([2H])c(-c4c5ccccc5c(-c5c([2H])c([2H])c([2H])c6c5oc5c([2H])c([2H])c([2H])c([2H])c56)c5ccccc45)c([2H])c([2H])c3c2[2H])c([2H])c1[2H] ; [2H]c1c([2H])c([2H])c(-c2c([2H])c([2H])c3c([2H])c(-c4c5ccccc5c(-c5c([2H])c([2H])c6c(oc7c([2H])c([2H])c([2H])c([2H])c76)c5[2H])c5ccccc45)c([2H])c([2H])c3c2[2H])c([2H])c1[2H]]

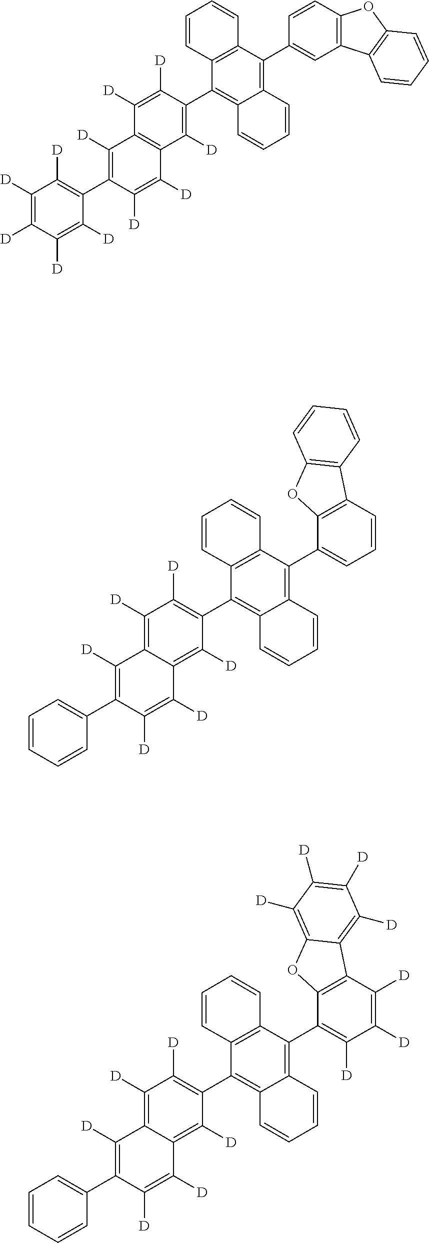 [SMILES: [2H]c1c([2H])c([2H])c(-c2c([2H])c([2H])c3c([2H])c(-c4c5ccccc5c(-c5ccc6oc7ccccc7c6c5)c5ccccc45)c([2H])c([2H])c3c2[2H])c([2H])c1[2H] ; [2H]c1c(-c2c3ccccc3c(-c3cccc4c3oc3ccccc34)c3ccccc23)c([2H])c2c([2H])c([2H])c(-c3ccccc3)c([2H])c2c1[2H] ; [2H]c1c([2H])c([2H])c2c(oc3c(-c4c5ccccc5c(-c5c([2H])c([2H])c6c([2H])c(-c7ccccc7)c([2H])c([2H])c6c5[2H])c5ccccc45)c([2H])c([2H])c([2H])c32)c1[2H]]

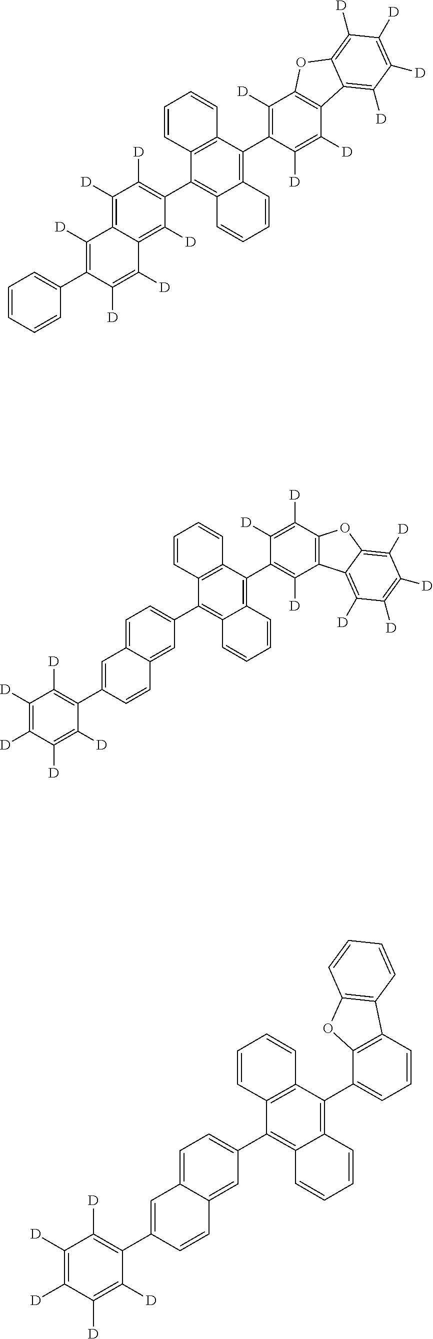 [SMILES: [2H]c1c([2H])c([2H])c2c(oc3c([2H])c(-c4c5ccccc5c(-c5c([2H])c([2H])c6c([2H])c(-c7ccccc7)c([2H])c([2H])c6c5[2H])c5ccccc45)c([2H])c([2H])c32)c1[2H] ; [2H]c1c([2H])c([2H])c(-c2ccc3cc(-c4c5ccccc5c(-c5c([2H])c([2H])c6oc7c([2H])c([2H])c([2H])c([2H])c7c6c5[2H])c5ccccc45)ccc3c2)c([2H])c1[2H] ; [2H]c1c([2H])c([2H])c(-c2ccc3cc(-c4c5ccccc5c(-c5cccc6c5oc5ccccc56)c5ccccc45)ccc3c2)c([2H])c1[2H]]

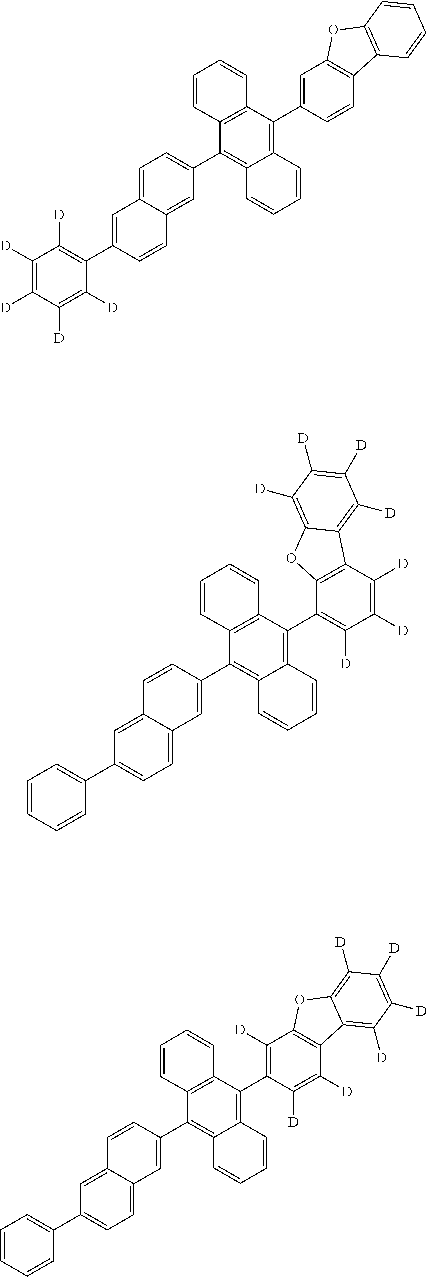 [SMILES: [2H]c1c([2H])c([2H])c(-c2ccc3cc(-c4c5ccccc5c(-c5ccc6c(c5)oc5ccccc56)c5ccccc45)ccc3c2)c([2H])c1[2H] ; [2H]c1c([2H])c([2H])c2c(oc3c(-c4c5ccccc5c(-c5ccc6cc(-c7ccccc7)ccc6c5)c5ccccc45)c([2H])c([2H])c([2H])c32)c1[2H] ; [2H]c1c([2H])c([2H])c2c(oc3c([2H])c(-c4c5ccccc5c(-c5ccc6cc(-c7ccccc7)ccc6c5)c5ccccc45)c([2H])c([2H])c32)c1[2H]]

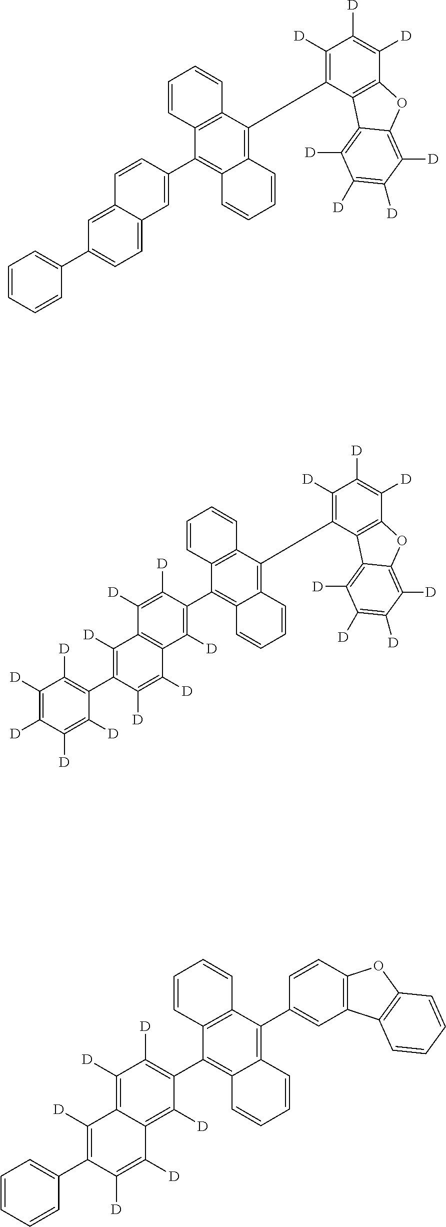 [SMILES: [2H]c1c([2H])c([2H])c2c(oc3c([2H])c([2H])c([2H])c(-c4c5ccccc5c(-c5ccc6cc(-c7ccccc7)ccc6c5)c5ccccc45)c32)c1[2H] ; [2H]c1c([2H])c([2H])c(-c2c([2H])c([2H])c3c([2H])c(-c4c5ccccc5c(-c5c([2H])c([2H])c([2H])c6oc7c([2H])c([2H])c([2H])c([2H])c7c56)c5ccccc45)c([2H])c([2H])c3c2[2H])c([2H])c1[2H] ; [2H]c1c(-c2c3ccccc3c(-c3ccc4oc5ccccc5c4c3)c3ccccc23)c([2H])c2c([2H])c([2H])c(-c3ccccc3)c([2H])c2c1[2H]]

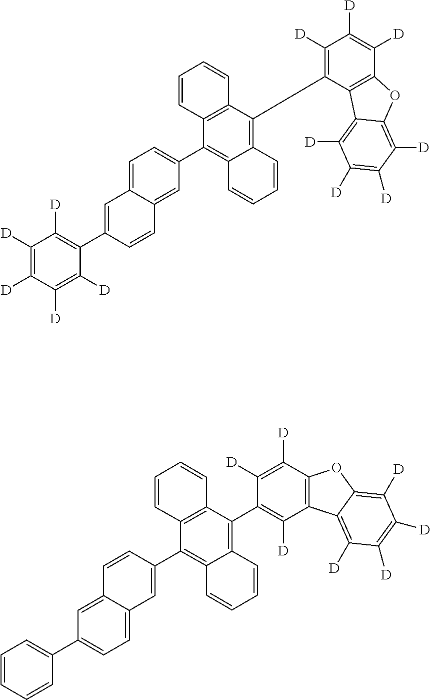 [SMILES: [2H]c1c([2H])c([2H])c(-c2ccc3cc(-c4c5ccccc5c(-c5c([2H])c([2H])c([2H])c6oc7c([2H])c([2H])c([2H])c([2H])c7c56)c5ccccc45)ccc3c2)c([2H])c1[2H] ; [2H]c1c([2H])c([2H])c2c(oc3c([2H])c([2H])c(-c4c5ccccc5c(-c5ccc6cc(-c7ccccc7)ccc6c5)c5ccccc45)c([2H])c32)c1[2H]]

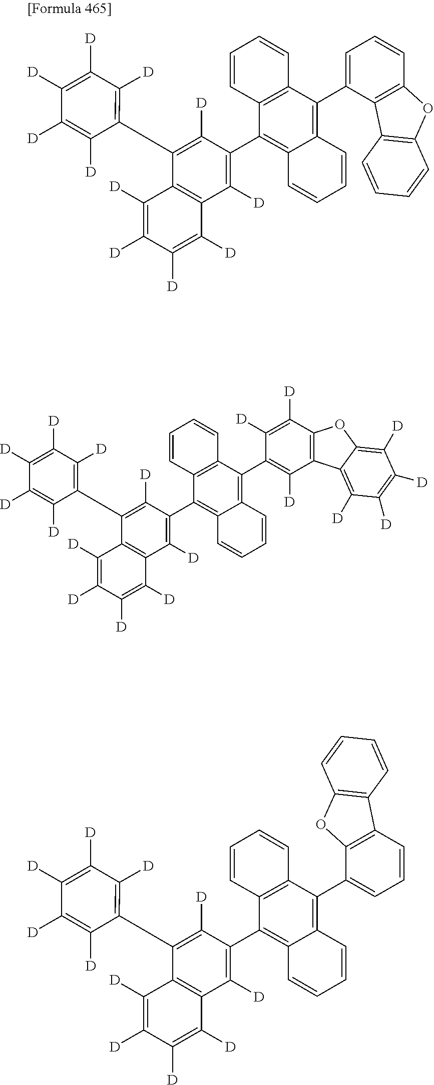 [SMILES: [2H]c1c([2H])c([2H])c(-c2c([2H])c(-c3c4ccccc4c(-c4cccc5oc6ccccc6c45)c4ccccc34)c([2H])c3c([2H])c([2H])c([2H])c([2H])c23)c([2H])c1[2H] ; [2H]c1c([2H])c([2H])c(-c2c([2H])c(-c3c4ccccc4c(-c4c([2H])c([2H])c5oc6c([2H])c([2H])c([2H])c([2H])c6c5c4[2H])c4ccccc34)c([2H])c3c([2H])c([2H])c([2H])c([2H])c23)c([2H])c1[2H] ; [2H]c1c([2H])c([2H])c(-c2c([2H])c(-c3c4ccccc4c(-c4cccc5c4oc4ccccc45)c4ccccc34)c([2H])c3c([2H])c([2H])c([2H])c([2H])c23)c([2H])c1[2H]]

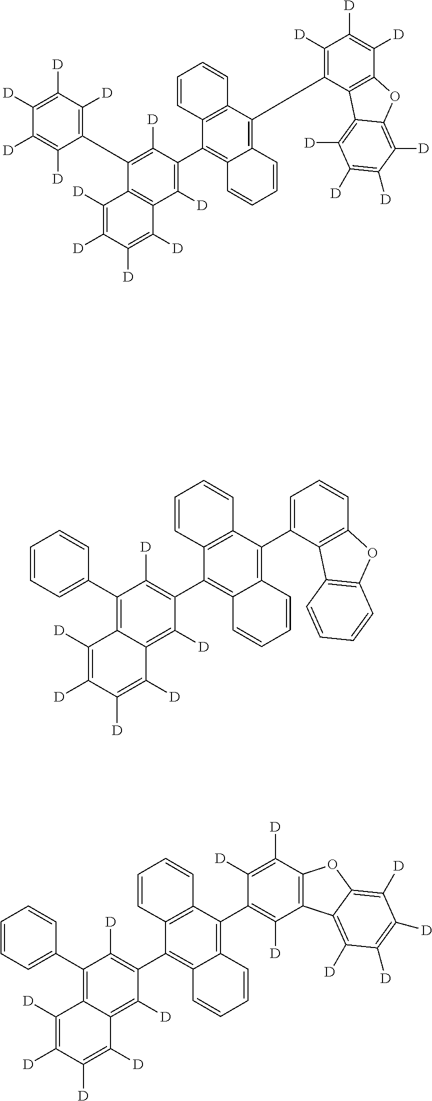 [SMILES: [2H]c1c([2H])c([2H])c(-c2c([2H])c(-c3c4ccccc4c(-c4c([2H])c([2H])c([2H])c5oc6c([2H])c([2H])c([2H])c([2H])c6c45)c4ccccc34)c([2H])c3c([2H])c([2H])c([2H])c([2H])c23)c([2H])c1[2H] ; [2H]c1c([2H])c([2H])c2c(-c3ccccc3)c([2H])c(-c3c4ccccc4c(-c4cccc5oc6ccccc6c45)c4ccccc34)c([2H])c2c1[2H] ; [2H]c1c([2H])c([2H])c2c(oc3c([2H])c([2H])c(-c4c5ccccc5c(-c5c([2H])c(-c6ccccc6)c6c([2H])c([2H])c([2H])c([2H])c6c5[2H])c5ccccc45)c([2H])c32)c1[2H]]

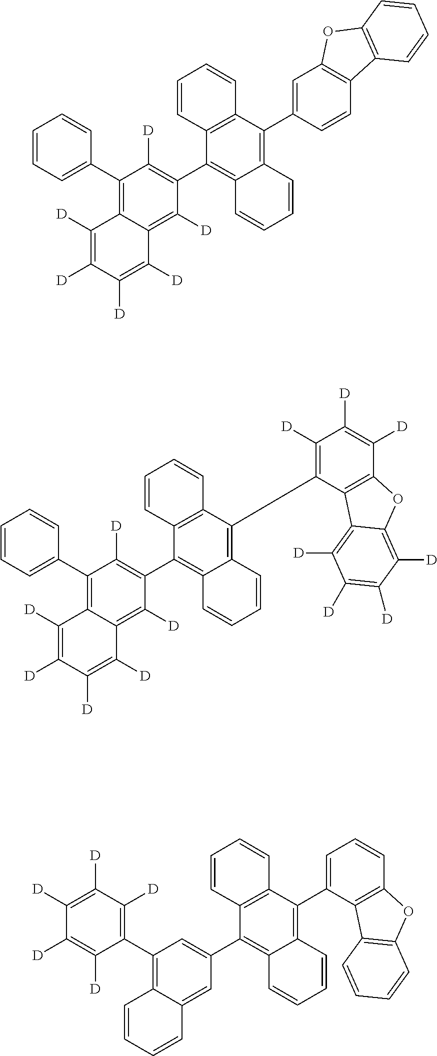 [SMILES: [2H]c1c([2H])c([2H])c2c(-c3ccccc3)c([2H])c(-c3c4ccccc4c(-c4ccc5c(c4)oc4ccccc45)c4ccccc34)c([2H])c2c1[2H] ; [2H]c1c([2H])c([2H])c2c(oc3c([2H])c([2H])c([2H])c(-c4c5ccccc5c(-c5c([2H])c(-c6ccccc6)c6c([2H])c([2H])c([2H])c([2H])c6c5[2H])c5ccccc45)c32)c1[2H] ; [2H]c1c([2H])c([2H])c(-c2cc(-c3c4ccccc4c(-c4cccc5oc6ccccc6c45)c4ccccc34)cc3ccccc23)c([2H])c1[2H]]

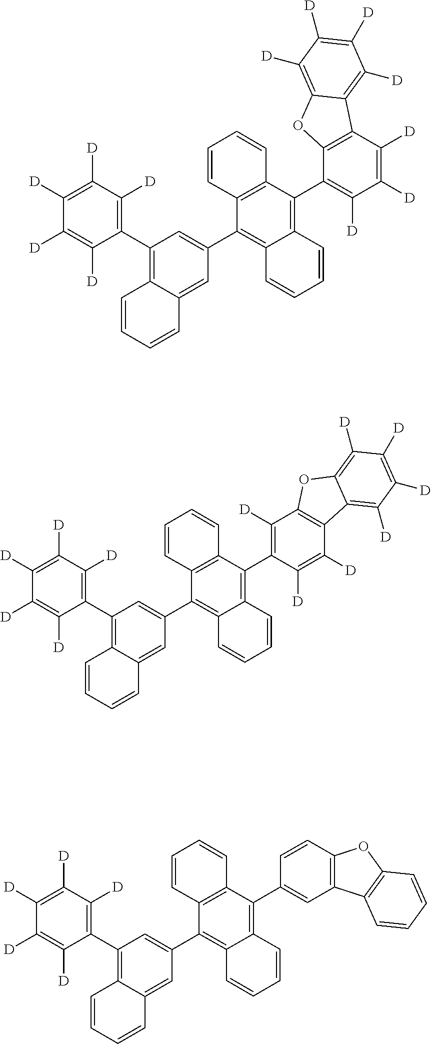 [SMILES: [2H]c1c([2H])c([2H])c(-c2cc(-c3c4ccccc4c(-c4c([2H])c([2H])c([2H])c5c4oc4c([2H])c([2H])c([2H])c([2H])c45)c4ccccc34)cc3ccccc23)c([2H])c1[2H] ; [2H]c1c([2H])c([2H])c(-c2cc(-c3c4ccccc4c(-c4c([2H])c([2H])c5c(oc6c([2H])c([2H])c([2H])c([2H])c65)c4[2H])c4ccccc34)cc3ccccc23)c([2H])c1[2H] ; [2H]c1c([2H])c([2H])c(-c2cc(-c3c4ccccc4c(-c4ccc5oc6ccccc6c5c4)c4ccccc34)cc3ccccc23)c([2H])c1[2H]]

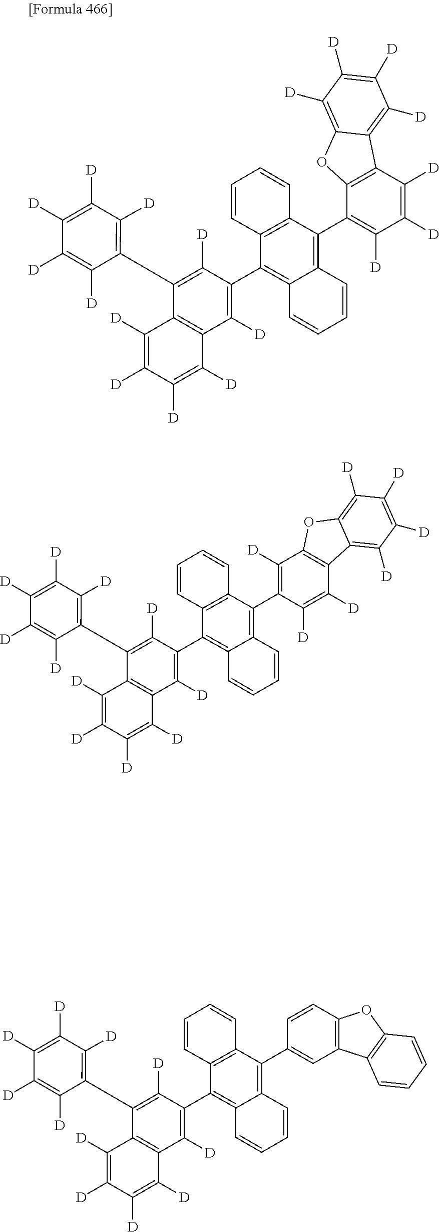 [SMILES: [2H]c1c([2H])c([2H])c(-c2c([2H])c(-c3c4ccccc4c(-c4c([2H])c([2H])c([2H])c5c4oc4c([2H])c([2H])c([2H])c([2H])c45)c4ccccc34)c([2H])c3c([2H])c([2H])c([2H])c([2H])c23)c([2H])c1[2H] ; [2H]c1c([2H])c([2H])c(-c2c([2H])c(-c3c4ccccc4c(-c4c([2H])c([2H])c5c(oc6c([2H])c([2H])c([2H])c([2H])c65)c4[2H])c4ccccc34)c([2H])c3c([2H])c([2H])c([2H])c([2H])c23)c([2H])c1[2H] ; [2H]c1c([2H])c([2H])c(-c2c([2H])c(-c3c4ccccc4c(-c4ccc5oc6ccccc6c5c4)c4ccccc34)c([2H])c3c([2H])c([2H])c([2H])c([2H])c23)c([2H])c1[2H]]

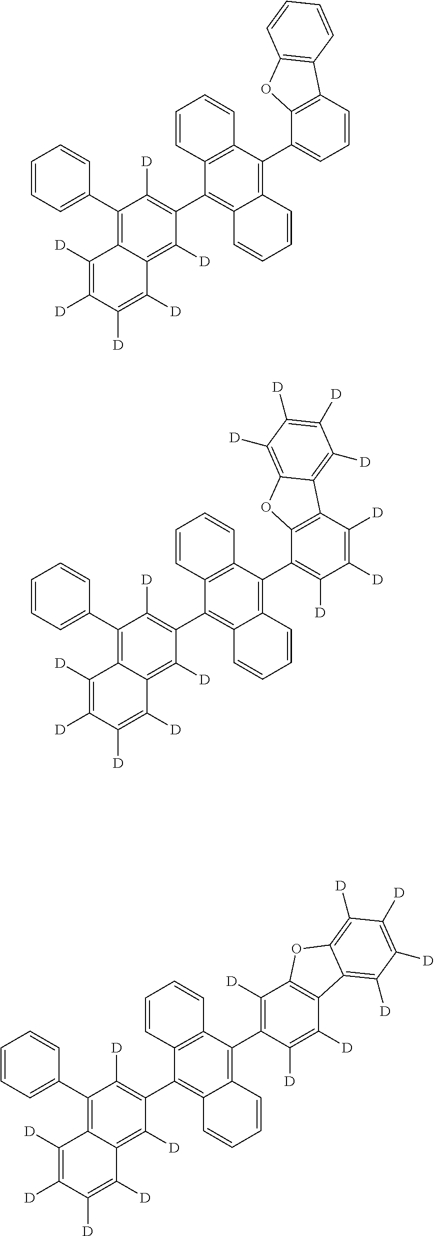 [SMILES: [2H]c1c([2H])c([2H])c2c(-c3ccccc3)c([2H])c(-c3c4ccccc4c(-c4cccc5c4oc4ccccc45)c4ccccc34)c([2H])c2c1[2H] ; [2H]c1c([2H])c([2H])c2c(oc3c(-c4c5ccccc5c(-c5c([2H])c(-c6ccccc6)c6c([2H])c([2H])c([2H])c([2H])c6c5[2H])c5ccccc45)c([2H])c([2H])c([2H])c32)c1[2H] ; [2H]c1c([2H])c([2H])c2c(oc3c([2H])c(-c4c5ccccc5c(-c5c([2H])c(-c6ccccc6)c6c([2H])c([2H])c([2H])c([2H])c6c5[2H])c5ccccc45)c([2H])c([2H])c32)c1[2H]]

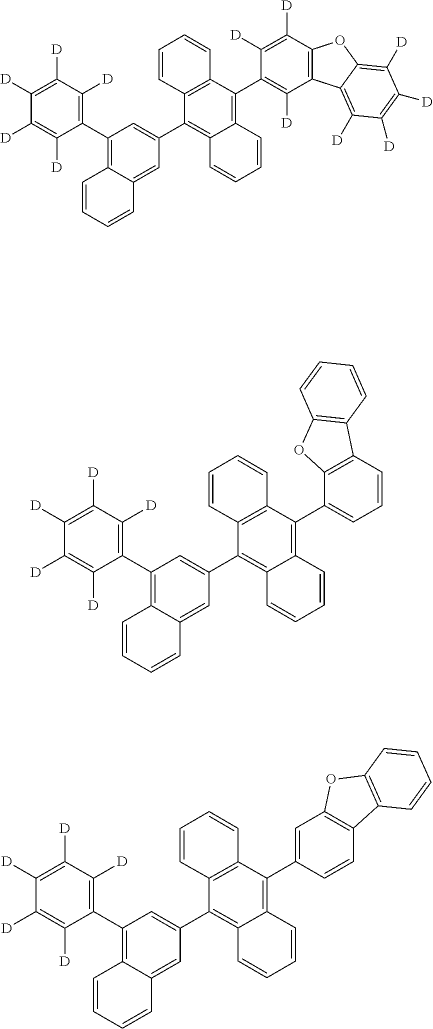 [SMILES: [2H]c1c([2H])c([2H])c(-c2cc(-c3c4ccccc4c(-c4c([2H])c([2H])c5oc6c([2H])c([2H])c([2H])c([2H])c6c5c4[2H])c4ccccc34)cc3ccccc23)c([2H])c1[2H] ; [2H]c1c([2H])c([2H])c(-c2cc(-c3c4ccccc4c(-c4cccc5c4oc4ccccc45)c4ccccc34)cc3ccccc23)c([2H])c1[2H] ; [2H]c1c([2H])c([2H])c(-c2cc(-c3c4ccccc4c(-c4ccc5c(c4)oc4ccccc45)c4ccccc34)cc3ccccc23)c([2H])c1[2H]]

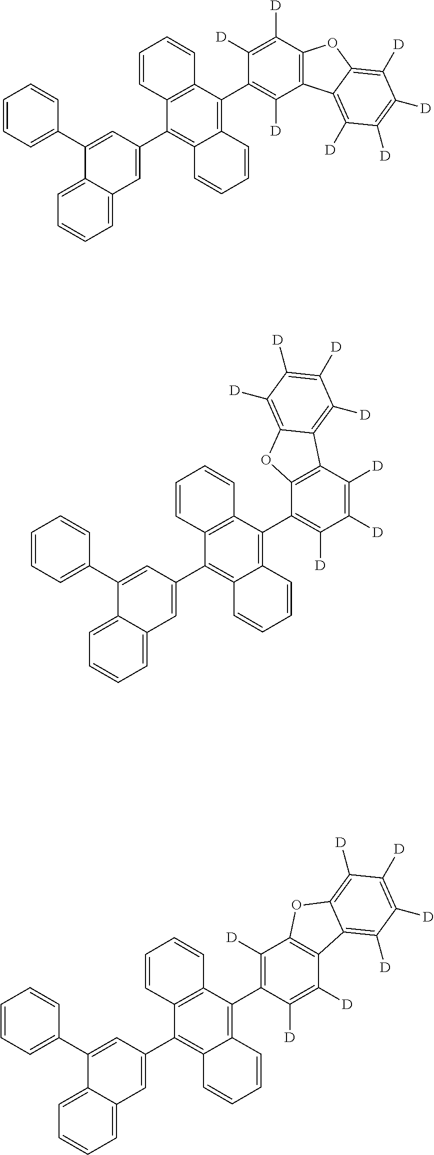 [SMILES: [2H]c1c([2H])c([2H])c2c(oc3c([2H])c([2H])c(-c4c5ccccc5c(-c5cc(-c6ccccc6)c6ccccc6c5)c5ccccc45)c([2H])c32)c1[2H] ; [2H]c1c([2H])c([2H])c2c(oc3c(-c4c5ccccc5c(-c5cc(-c6ccccc6)c6ccccc6c5)c5ccccc45)c([2H])c([2H])c([2H])c32)c1[2H] ; [2H]c1c([2H])c([2H])c2c(oc3c([2H])c(-c4c5ccccc5c(-c5cc(-c6ccccc6)c6ccccc6c5)c5ccccc45)c([2H])c([2H])c32)c1[2H]]

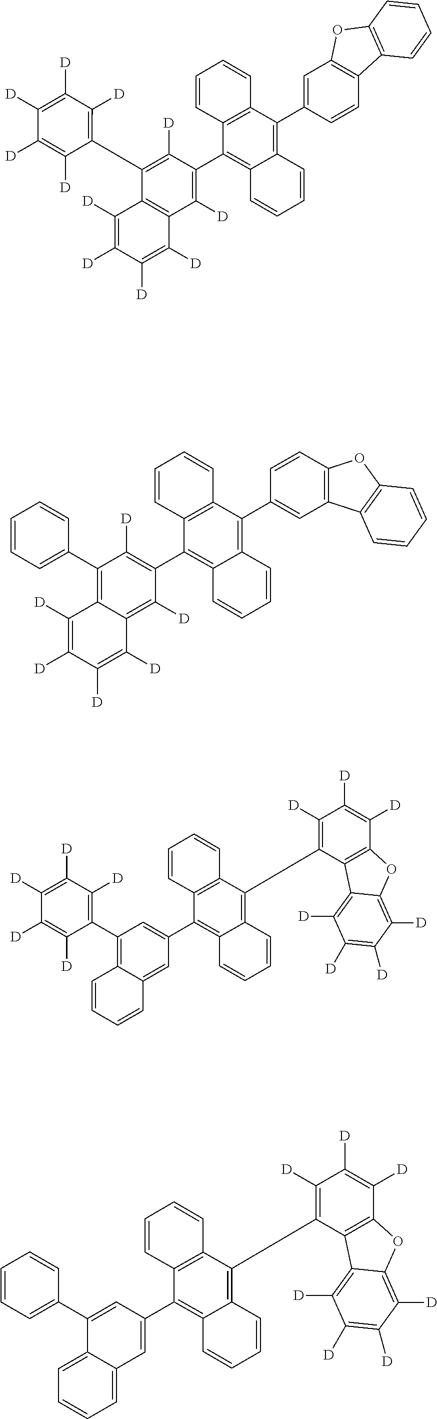 [SMILES: [2H]c1c([2H])c([2H])c(-c2c([2H])c(-c3c4ccccc4c(-c4ccc5c(c4)oc4ccccc45)c4ccccc34)c([2H])c3c([2H])c([2H])c([2H])c([2H])c23)c([2H])c1[2H] ; [2H]c1c([2H])c([2H])c2c(-c3ccccc3)c([2H])c(-c3c4ccccc4c(-c4ccc5oc6ccccc6c5c4)c4ccccc34)c([2H])c2c1[2H] ; [2H]c1c([2H])c([2H])c(-c2cc(-c3c4ccccc4c(-c4c([2H])c([2H])c([2H])c5oc6c([2H])c([2H])c([2H])c([2H])c6c45)c4ccccc34)cc3ccccc23)c([2H])c1[2H] ; [2H]c1c([2H])c([2H])c2c(oc3c([2H])c([2H])c([2H])c(-c4c5ccccc5c(-c5cc(-c6ccccc6)c6ccccc6c5)c5ccccc45)c32)c1[2H]]

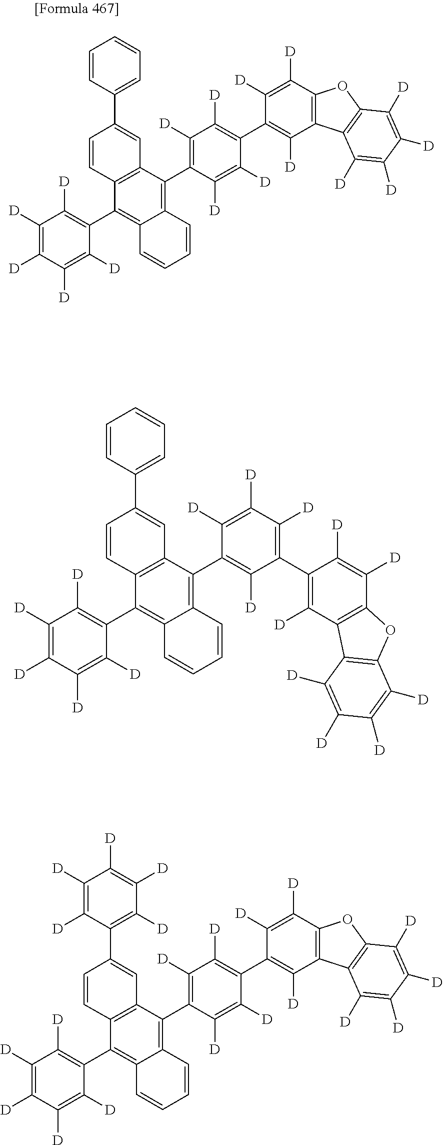

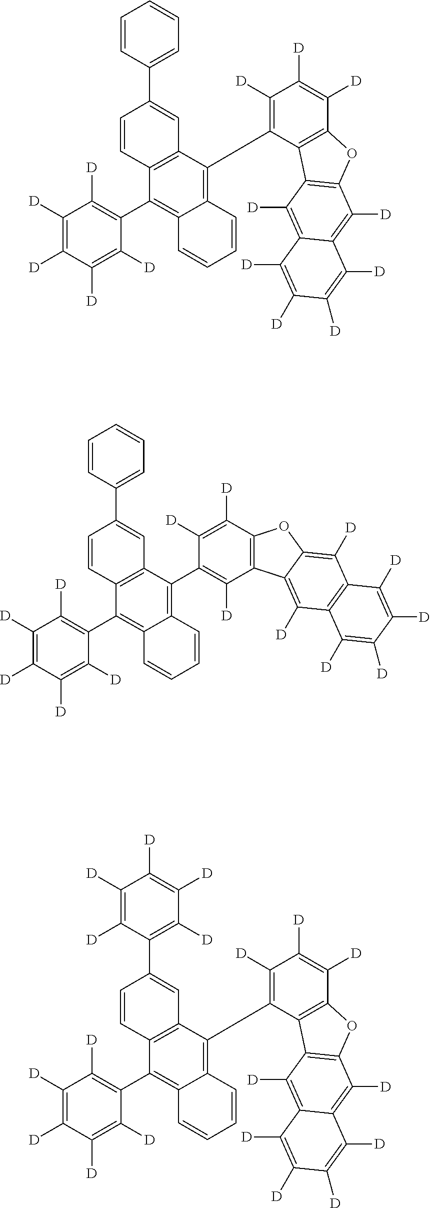 [SMILES: [2H]c1c([2H])c([2H])c(-c2c3ccccc3c(-c3c([2H])c([2H])c([2H])c4oc5c([2H])c6c([2H])c([2H])c([2H])c([2H])c6c([2H])c5c34)c3cc(-c4ccccc4)ccc23)c([2H])c1[2H] ; [2H]c1c([2H])c([2H])c(-c2c3ccccc3c(-c3c([2H])c([2H])c4oc5c([2H])c6c([2H])c([2H])c([2H])c([2H])c6c([2H])c5c4c3[2H])c3cc(-c4ccccc4)ccc23)c([2H])c1[2H] ; [2H]c1c([2H])c([2H])c(-c2ccc3c(-c4c([2H])c([2H])c([2H])c([2H])c4[2H])c4ccccc4c(-c4c([2H])c([2H])c([2H])c5oc6c([2H])c7c([2H])c([2H])c([2H])c([2H])c7c([2H])c6c45)c3c2)c([2H])c1[2H]]

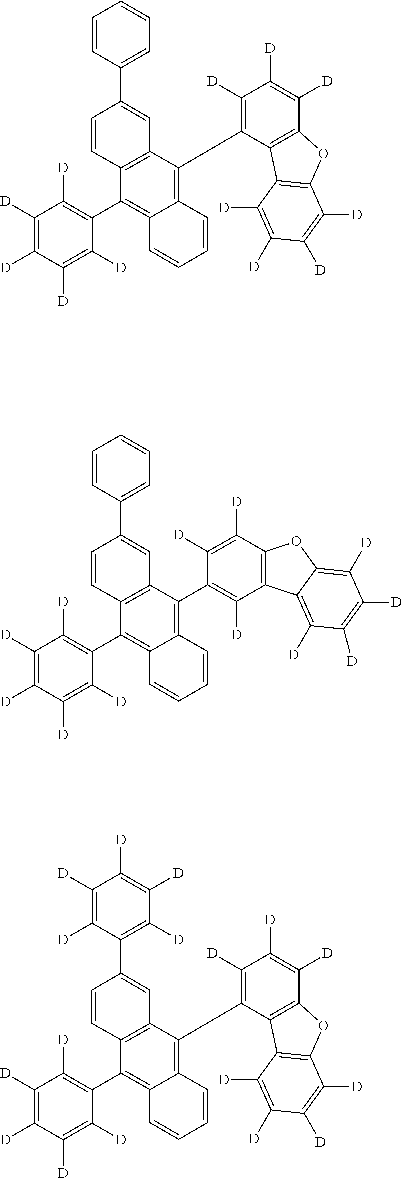 [SMILES: [2H]c1c([2H])c([2H])c(-c2c3ccccc3c(-c3c([2H])c([2H])c([2H])c4oc5c([2H])c([2H])c([2H])c([2H])c5c34)c3cc(-c4ccccc4)ccc23)c([2H])c1[2H] ; [2H]c1c([2H])c([2H])c(-c2c3ccccc3c(-c3c([2H])c([2H])c4oc5c([2H])c([2H])c([2H])c([2H])c5c4c3[2H])c3cc(-c4ccccc4)ccc23)c([2H])c1[2H] ; [2H]c1c([2H])c([2H])c(-c2ccc3c(-c4c([2H])c([2H])c([2H])c([2H])c4[2H])c4ccccc4c(-c4c([2H])c([2H])c([2H])c5oc6c([2H])c([2H])c([2H])c([2H])c6c45)c3c2)c([2H])c1[2H]]

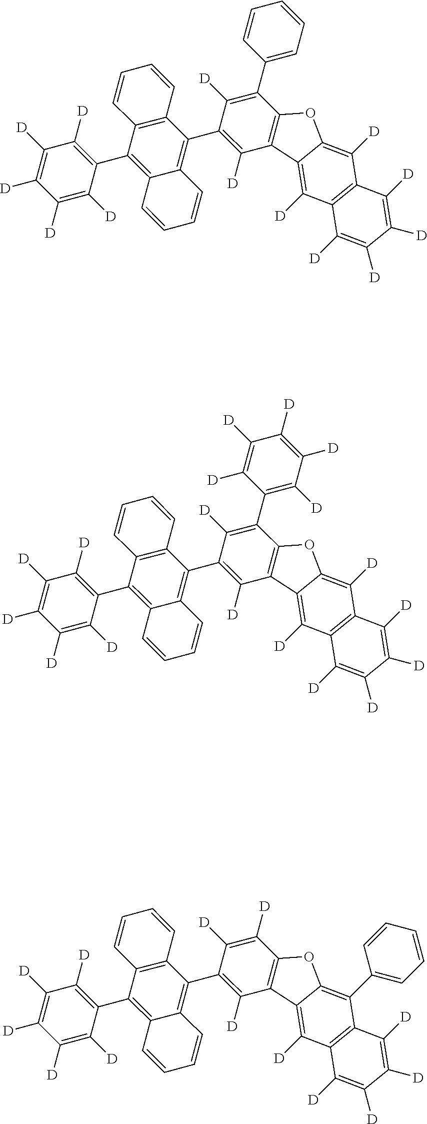 [SMILES: [2H]c1c([2H])c([2H])c(-c2c3ccccc3c(-c3c([2H])c(-c4ccccc4)c4oc5c([2H])c6c([2H])c([2H])c([2H])c([2H])c6c([2H])c5c4c3[2H])c3ccccc23)c([2H])c1[2H] ; [2H]c1c([2H])c([2H])c(-c2c3ccccc3c(-c3c([2H])c(-c4c([2H])c([2H])c([2H])c([2H])c4[2H])c4oc5c([2H])c6c([2H])c([2H])c([2H])c([2H])c6c([2H])c5c4c3[2H])c3ccccc23)c([2H])c1[2H] ; [2H]c1c([2H])c([2H])c(-c2c3ccccc3c(-c3c([2H])c([2H])c4oc5c(-c6ccccc6)c6c([2H])c([2H])c([2H])c([2H])c6c([2H])c5c4c3[2H])c3ccccc23)c([2H])c1[2H]]

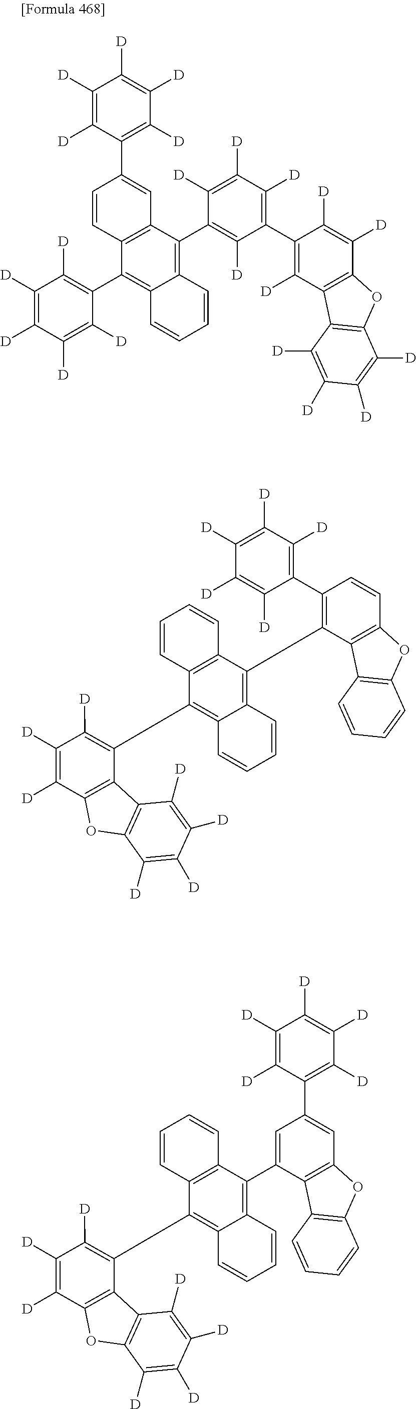 [SMILES: [2H]c1c([2H])c([2H])c(-c2ccc3c(-c4c([2H])c([2H])c([2H])c([2H])c4[2H])c4ccccc4c(-c4c([2H])c([2H])c([2H])c(-c5c([2H])c([2H])c6oc7c([2H])c([2H])c([2H])c([2H])c7c6c5[2H])c4[2H])c3c2)c([2H])c1[2H] ; [2H]c1c([2H])c([2H])c(-c2ccc3oc4ccccc4c3c2-c2c3ccccc3c(-c3c([2H])c([2H])c([2H])c4oc5c([2H])c([2H])c([2H])c([2H])c5c34)c3ccccc23)c([2H])c1[2H] ; [2H]c1c([2H])c([2H])c(-c2cc(-c3c4ccccc4c(-c4c([2H])c([2H])c([2H])c5oc6c([2H])c([2H])c([2H])c([2H])c6c45)c4ccccc34)c3c(c2)oc2ccccc23)c([2H])c1[2H]]

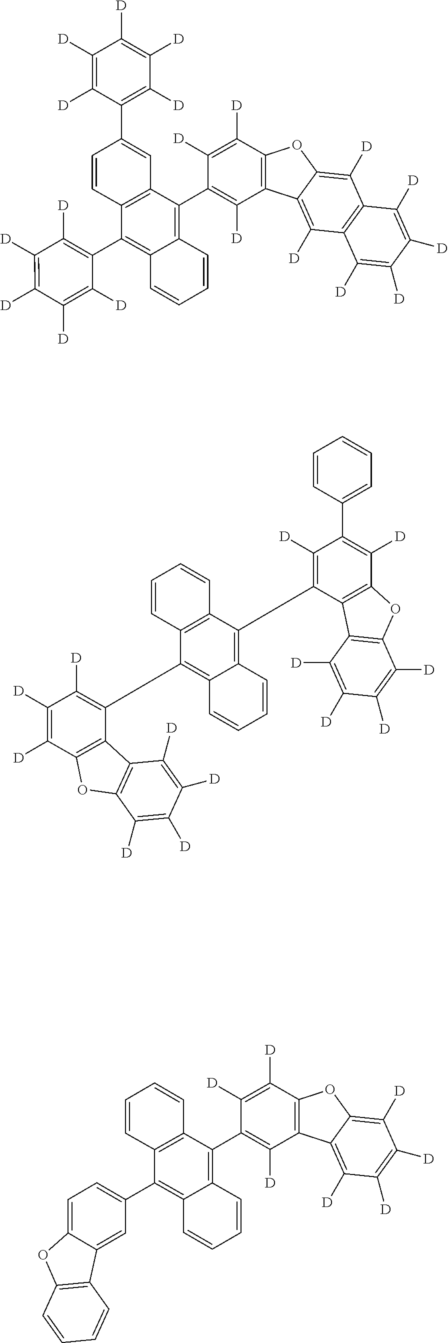 [SMILES: [2H]c1c([2H])c([2H])c(-c2ccc3c(-c4c([2H])c([2H])c([2H])c([2H])c4[2H])c4ccccc4c(-c4c([2H])c([2H])c5oc6c([2H])c7c([2H])c([2H])c([2H])c([2H])c7c([2H])c6c5c4[2H])c3c2)c([2H])c1[2H] ; [2H]c1c([2H])c([2H])c2c(oc3c([2H])c([2H])c([2H])c(-c4c5ccccc5c(-c5c([2H])c(-c6ccccc6)c([2H])c6oc7c([2H])c([2H])c([2H])c([2H])c7c56)c5ccccc45)c32)c1[2H] ; [2H]c1c([2H])c([2H])c2c(oc3c([2H])c([2H])c(-c4c5ccccc5c(-c5ccc6oc7ccccc7c6c5)c5ccccc45)c([2H])c32)c1[2H]]

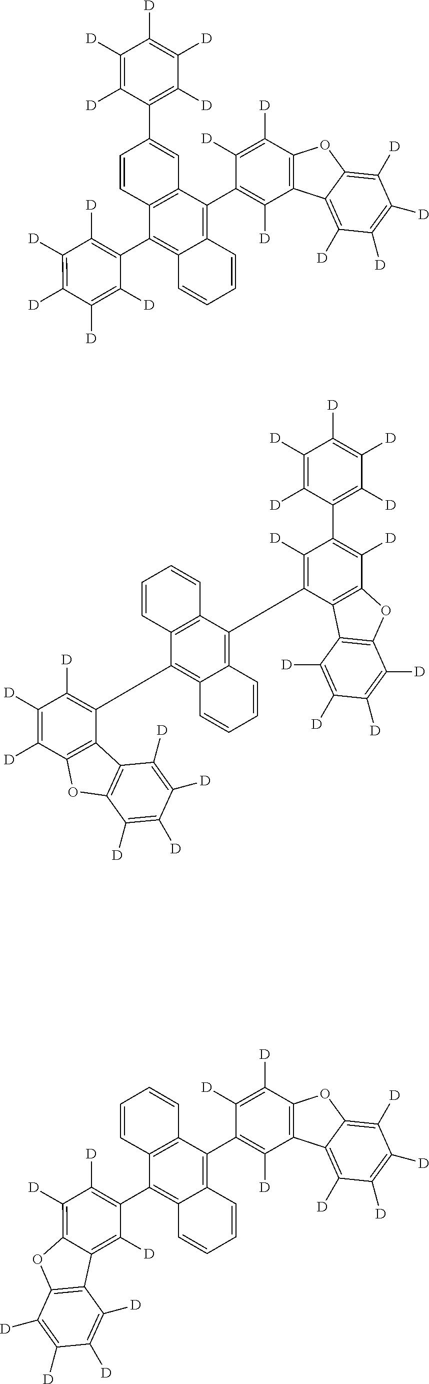 [SMILES: [2H]c1c([2H])c([2H])c(-c2ccc3c(-c4c([2H])c([2H])c([2H])c([2H])c4[2H])c4ccccc4c(-c4c([2H])c([2H])c5oc6c([2H])c([2H])c([2H])c([2H])c6c5c4[2H])c3c2)c([2H])c1[2H] ; [2H]c1c([2H])c([2H])c(-c2c([2H])c(-c3c4ccccc4c(-c4c([2H])c([2H])c([2H])c5oc6c([2H])c([2H])c([2H])c([2H])c6c45)c4ccccc34)c3c(oc4c([2H])c([2H])c([2H])c([2H])c43)c2[2H])c([2H])c1[2H] ; [2H]c1c([2H])c([2H])c2c(oc3c([2H])c([2H])c(-c4c5ccccc5c(-c5c([2H])c([2H])c6oc7c([2H])c([2H])c([2H])c([2H])c7c6c5[2H])c5ccccc45)c([2H])c32)c1[2H]]

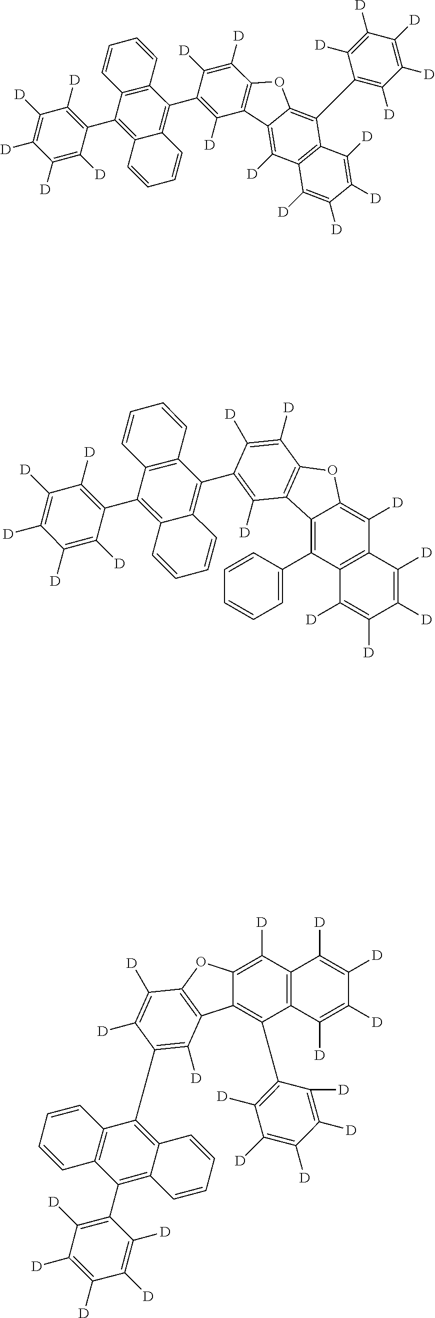 [SMILES: [2H]c1c([2H])c([2H])c(-c2c3ccccc3c(-c3c([2H])c([2H])c4oc5c(-c6c([2H])c([2H])c([2H])c([2H])c6[2H])c6c([2H])c([2H])c([2H])c([2H])c6c([2H])c5c4c3[2H])c3ccccc23)c([2H])c1[2H] ; [2H]c1c([2H])c([2H])c(-c2c3ccccc3c(-c3c([2H])c([2H])c4oc5c([2H])c6c([2H])c([2H])c([2H])c([2H])c6c(-c6ccccc6)c5c4c3[2H])c3ccccc23)c([2H])c1[2H] ; [2H]c1c([2H])c([2H])c(-c2c3ccccc3c(-c3c([2H])c([2H])c4oc5c([2H])c6c([2H])c([2H])c([2H])c([2H])c6c(-c6c([2H])c([2H])c([2H])c([2H])c6[2H])c5c4c3[2H])c3ccccc23)c([2H])c1[2H]]

### Third Compound and Fourth Compound

In the organic EL device according to the exemplary embodiment, it is also preferable that the first emitting layer further contains a third compound that emits fluorescence.

In the organic EL device according to the exemplary embodiment, it is also preferable that the second emitting layer further contains a fourth compound that emits fluorescence.

When the first emitting layer contains the third compound and the second emitting layer contains the fourth compound, the third compound and the fourth compound are mutually the same or different.

The third compound and the fourth compound are each independently at least one compound selected from the group consisting of a compound represented by a formula (3), a compound represented by a formula (4), a compound represented by a formula (5), a compound represented by a formula (6), a compound represented by a formula (7), a compound represented by a formula (8), a compound represented by a formula (9), and a compound represented by a formula (10).

### Compound Represented by Formula (3)

The compound represented by the formula (3) will be described.

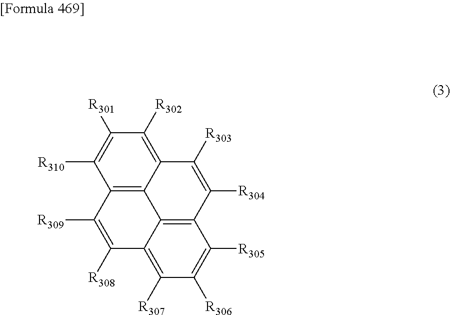

In the formula (3):
at least one combination of adjacent two or more of R₃₀₁ to R₃₁₀ are mutually bonded to form a substituted or unsubstituted monocyclic ring, mutually bonded to form a substituted or unsubstituted fused ring, or not mutually bonded;
at least one of R₃₀₁ to R₃₁₀ is a monovalent group represented by a formula (31) below; and
R₃₀₁ to R₃₁₀ forming neither the monocyclic ring nor the fused ring and not being the monovalent group represented by the formula (31) are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

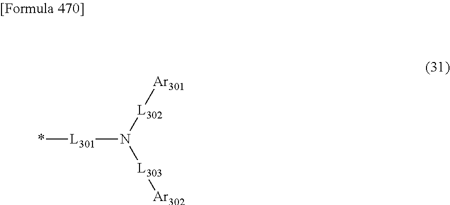

In the formula (31):
Ar₃₀₁ and Ar₃₀₂ are each independently a substituted or unsubstituted aryl group having 6 to 50 ring carbon atoms, or a substituted or unsubstituted heterocyclic group having 5 to 50 ring atoms;
L₃₀₁ to L₃₀₃ are each independently a single bond, a substituted or unsubstituted arylene group having 6 to 30 ring carbon atoms, or a substituted or unsubstituted divalent heterocyclic group having 5 to 30 ring atoms; and
* represents a bonding position to a pyrene ring in the formula (3).

In the third and fourth compounds, R₉₀₁, R₉₀₂, R₉₀₃, R₉₀₄, R₉₀₅, R₉₀₆, and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
a substituted or unsubstituted alkyl group having 1 to 50 carbon atoms, or a substituted or unsubstituted aryl group having 6 to 50 ring carbon atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

In the formula (3), it is preferable that two of R₃₀₁ to R₃₁₀ are each a group represented by the formula (31).

In an exemplary embodiment, the compound represented by the formula (3) is a compound represented by a formula (33) below.

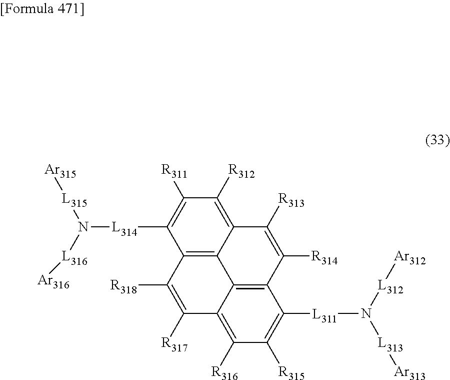

In the formula (33):
R₃₁₁ to R₃₁₈ each independently represent the same as R₃₀₁ to R₃₁₀ in the formula (3) that are not the monovalent group represented by the formula (31);
L₃₁₁ to L₃₁₆ are each independently a single bond, a substituted or unsubstituted arylene group having 6 to 30 ring carbon atoms, or a substituted or unsubstituted divalent heterocyclic group having 5 to 30 ring atoms; and
Ar₃₁₂, Ar₃₁₃, Ar₃₁₅, and Ar₃₁₆ are each independently a substituted or unsubstituted aryl group having 6 to 50 ring carbon atoms, or a substituted or unsubstituted heterocyclic group having 5 to 50 ring atoms.

In the formula (31), L₃₀₁ is preferably a single bond, and L₃₀₂ and L₃₀₃ are each preferably a single bond.

In an exemplary embodiment, the compound represented by the formula (3) is represented by a formula (34) or a formula (35) below.

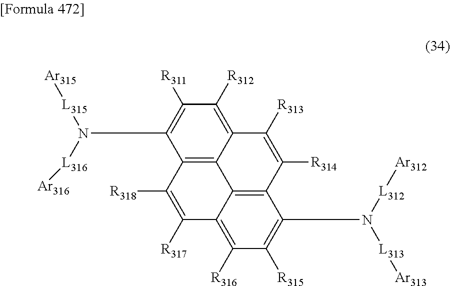

In the formula (34):
R₃₁₁ to R₃₁₈ each independently represent the same as R₃₀₁ to R₃₁₀ in the formula (3) that are not the monovalent group represented by the formula (31);
L₃₁₂, L₃₁₃, L₃₁₅ and L₃₁₆ each independently represent the same as L₃₁₂, L₃₁₃, L₃₁₅ and L₃₁₆ in the formula (33); and
Ar₃₁₂, Ar₃₁₃, Ar₃₁₅ and Ar₃₁A each independently represent the same as Ar₃₁₂, Ar₃₁₃, Ar₃₁₅ and Ar₃₁6 in the formula (33).

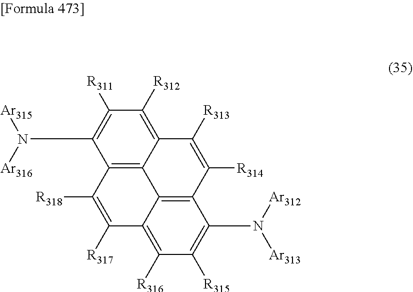

In the formula (35):
R₃₁₁ to R₃₁₈ each independently represent the same as R₃₀₁ to R₃₁₀ in the formula (3) that are not the monovalent group represented by the formula (31); and
Ar₃₁₂, Ar₃₁₃, Ar₃₁₅ and Ar₃₁₆ each independently represent the same as Ar₃₁₂, Ar₃₁₃, Ar₃₁₅ and Ar₃₁₆ in the formula (33).

In the formula (31), at least one of Ar₃₀₁ or Ar₃₀₂ is preferably a group represented by a formula (36) below.

In the formulae (33) to (35), at least one of Ar₃₁₂ or Ar₃₁₃ is preferably a group represented by the formula (36) below.

In the formulae (33) to (35), at least one of Ar₃₁₅ or Ar₃₁₆ is preferably a group represented by the formula (36) below.

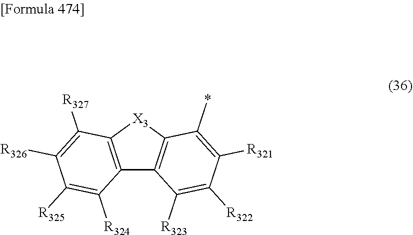

In the formula (36):
X₃ represents an oxygen atom or a sulfur atom;
at least one combination of adjacent two or more of R₃₂₁ to R₃₂₇ are mutually bonded to form a substituted or unsubstituted monocyclic ring, mutually bonded to form a substituted or unsubstituted fused ring, or not mutually bonded;
R₃₂₁ to R₃₂₇ forming neither the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
* represents a bonding position to L₃₀₂, L₃₀₃, L₃₁₂, L₃₁₃, L₃₁₅ or L₃₁₆.

X₃ is preferably an oxygen atom.

At least one of R₃₂₁ to R₃₂₇ is preferably a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the formula (31), it is preferable that Ar₃₀₁ is a group represented by the formula (36); and Ar₃₀₂ is a substituted or unsubstituted aryl group having 6 to 50 ring carbon atoms.

In the formulae (33) to (35), it is preferable that Ar₃₁₂ is a group represented by the formula (36); and Ar₃₁₃ is a substituted or unsubstituted aryl group having 6 to 50 ring carbon atoms.

In the formulae (33) to (35), it is preferable that Ar₃₁₅ is a group represented by the formula (36); and Ar₃₁₆ is a substituted or unsubstituted aryl group having 6 to 50 ring carbon atoms.

In an exemplary embodiment, the compound represented by the formula (3) is represented by a formula (37) below.

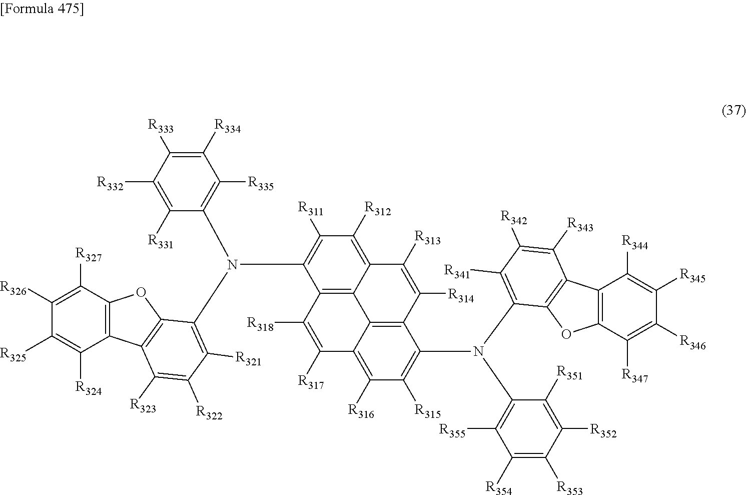

In the formula (37):
R₃₁₁ to R₃₁₃ each independently represent the same as R₃₀₁ to R₃₁₀ in the formula (3) that are not the monovalent group represented by the formula (31);
at least one combination of adjacent two or more of R₃₂₁ to R₃₂₇ are mutually bonded to form a substituted or unsubstituted monocyclic ring, mutually bonded to form a substituted or unsubstituted fused ring, or not mutually bonded;
at least one combination of adjacent two or more of R₃₄₁ to R₃₄₇ are mutually bonded to form a substituted or unsubstituted monocyclic ring, mutually bonded to form a substituted or unsubstituted fused ring, or not mutually bonded;
R₃₂₁ to R₃₂₇ and R₃₄₁ to R₃₄₇ forming neither the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
R₃₃₁ to R₃₃₅ and R₃₅₁ to R₃₅₅ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

Specific examples of the compound represented by the formula (3) include compounds shown below.

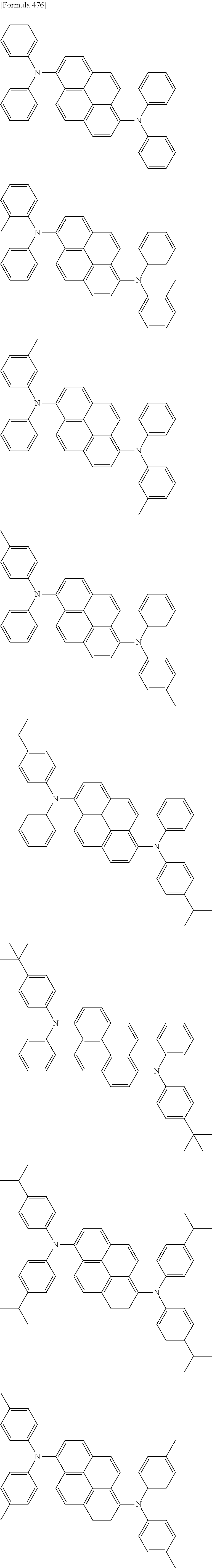

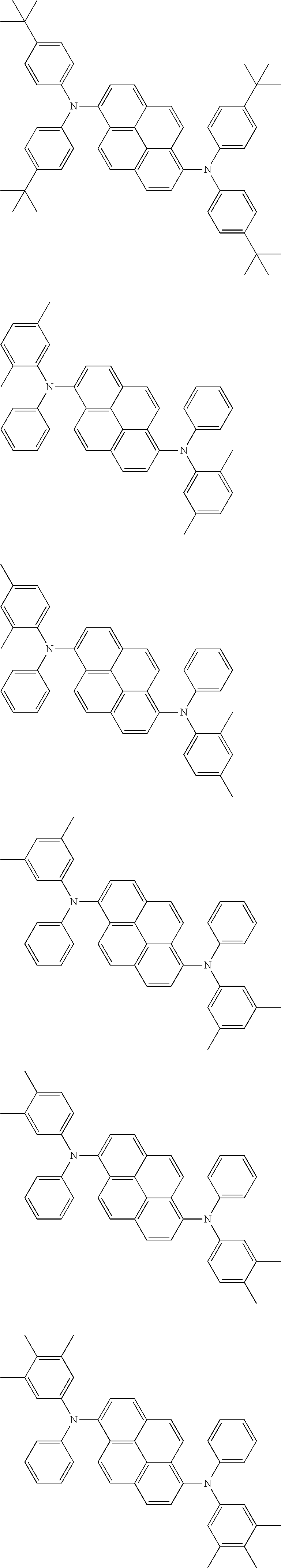

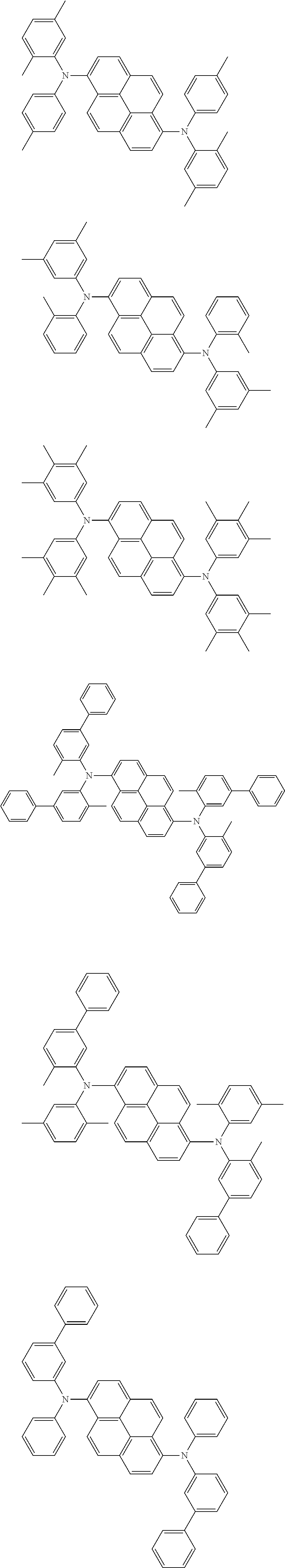

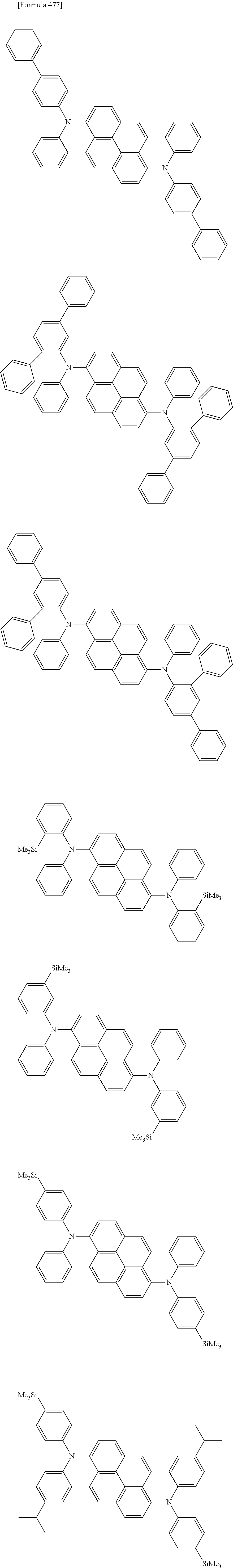

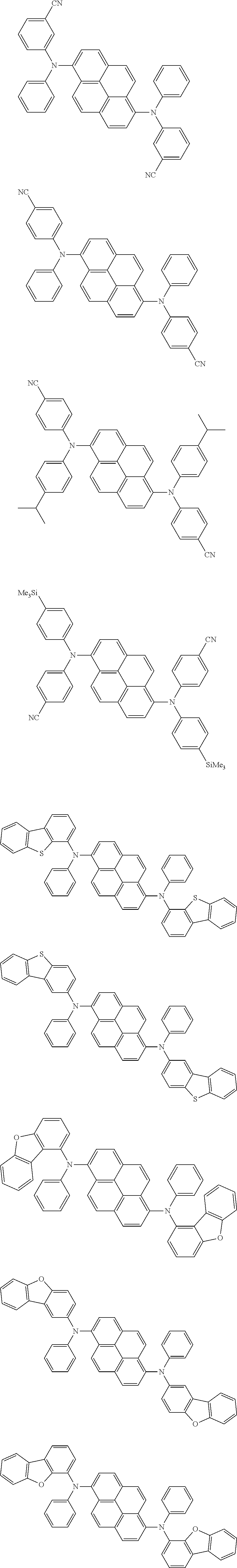

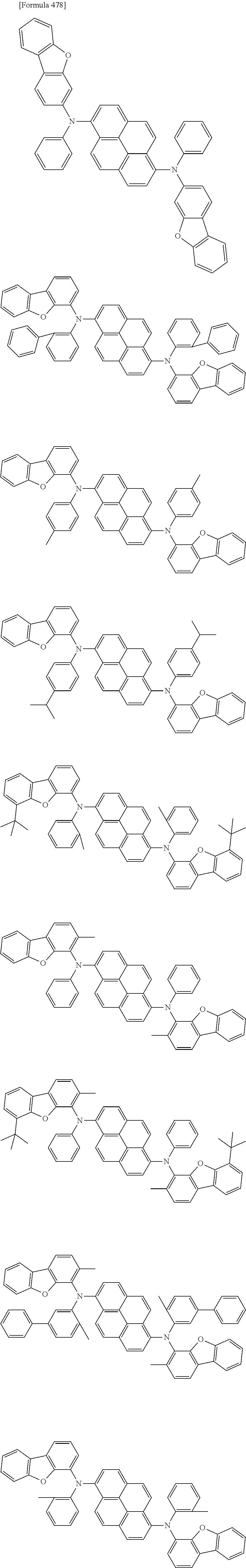

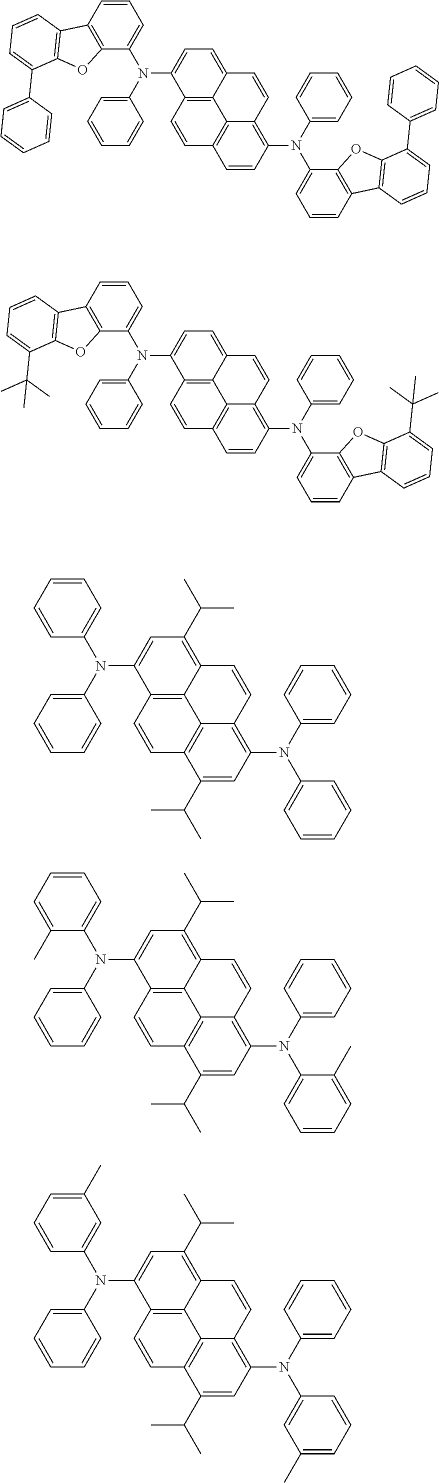

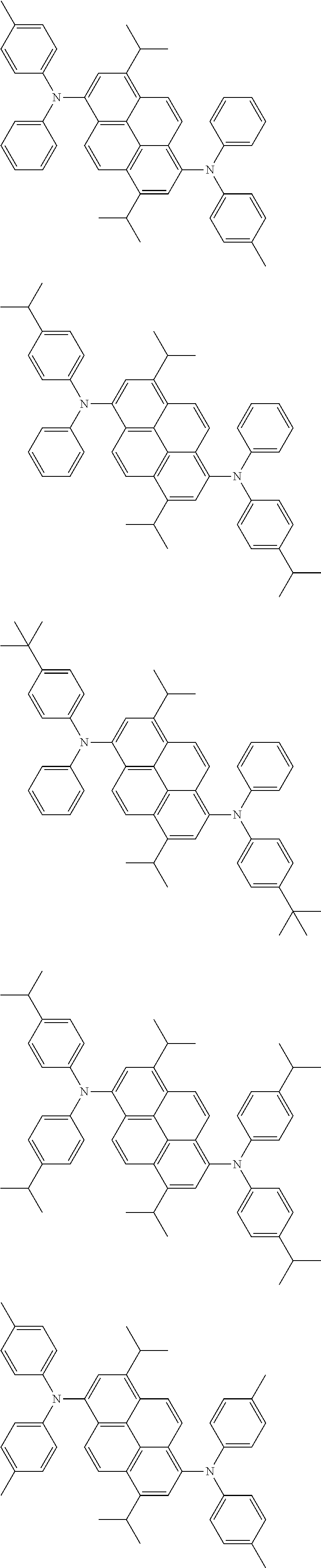

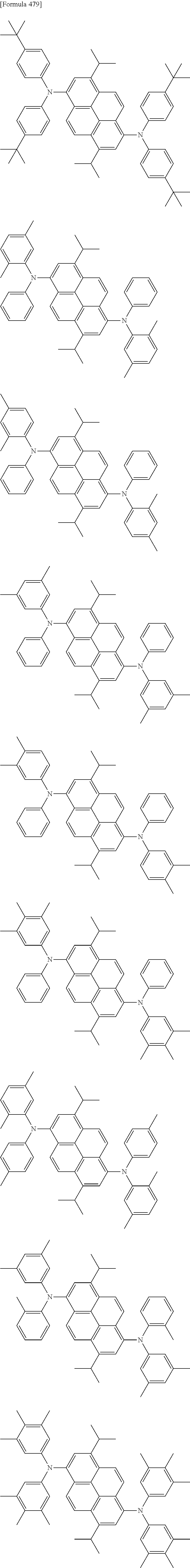

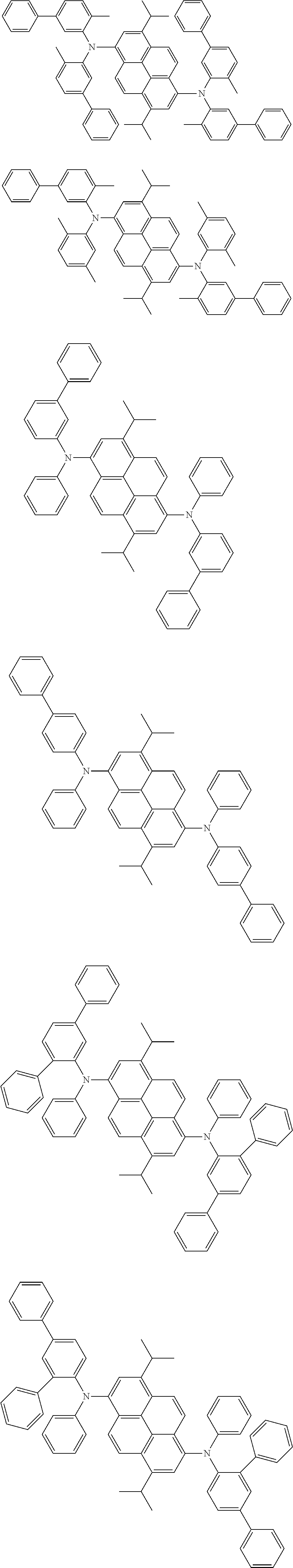

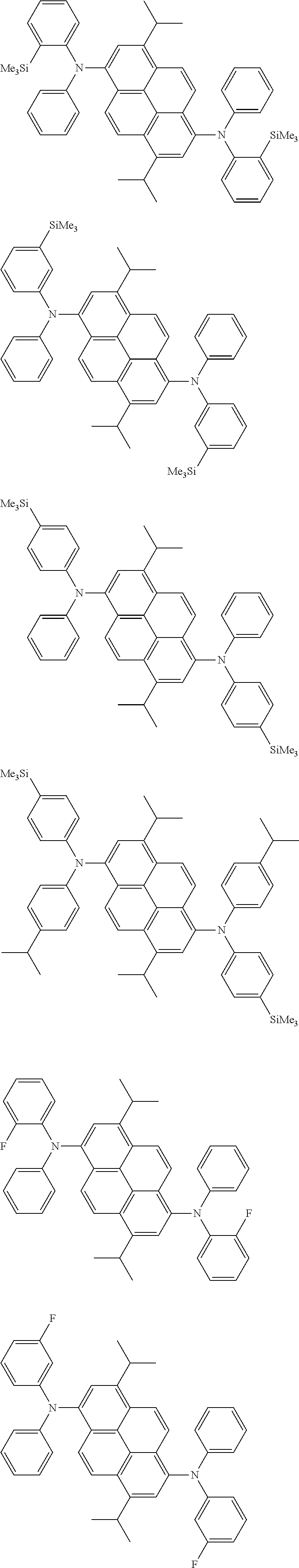

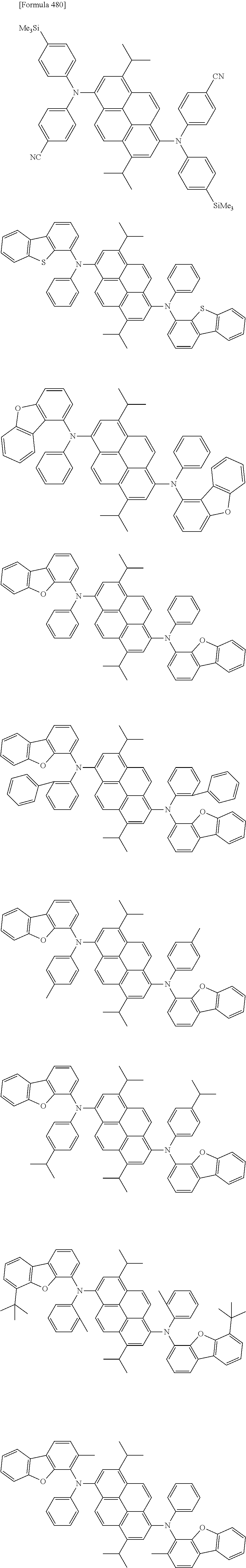

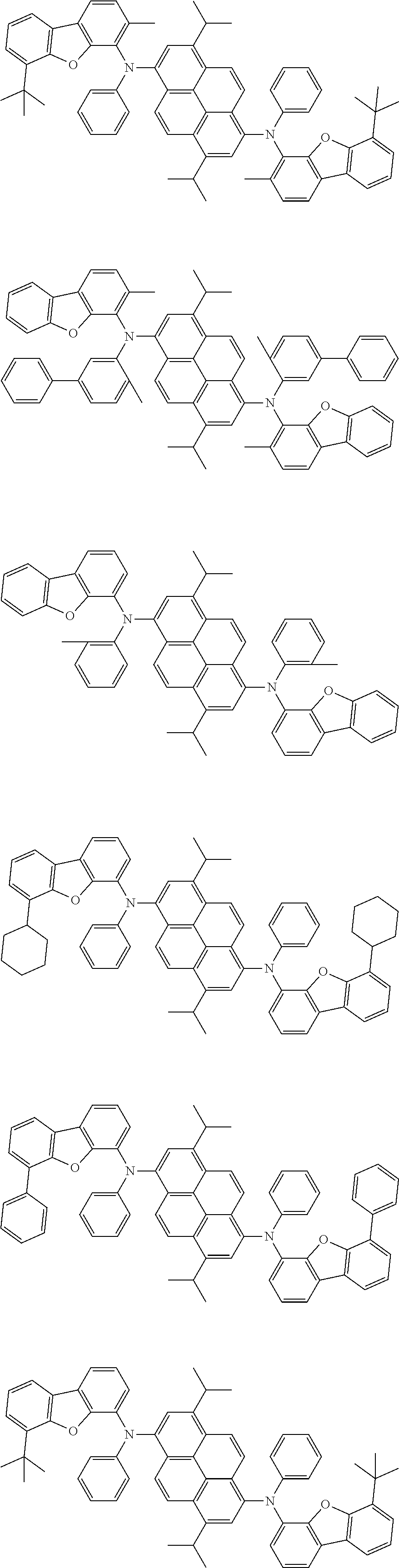

### Compound Represented by Formula (4)

The compound represented by the formula (4) will be described.

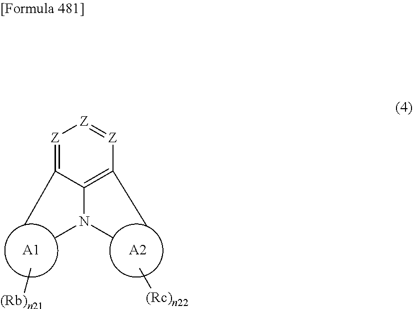

In the formula (4):
Z is each independently CRa or a nitrogen atom;
A1 ring and A2 ring are each independently a substituted or unsubstituted aromatic hydrocarbon ring having 6 to 50 ring carbon atoms, or a substituted or unsubstituted heterocycle having 5 to 50 ring atoms;
when a plurality of Ra are present, at least one combination of adjacent two or more of the plurality of Ra are mutually bonded to form a substituted or unsubstituted monocyclic ring, mutually bonded to form a substituted or unsubstituted fused ring, or not mutually bonded;
n21 and n22 are each independently 0, 1, 2, 3, or 4;
when a plurality of Rb are present, at least one combination of adjacent two or more of the plurality of Rb are mutually bonded to form a substituted or unsubstituted monocyclic ring, mutually bonded to form a substituted or unsubstituted fused ring, or not mutually bonded;
when a plurality of Rc are present, at least one combination of adjacent two or more of the plurality of Rc are mutually bonded to form a substituted or unsubstituted monocyclic ring, mutually bonded to form a substituted or unsubstituted fused ring, or not mutually bonded; and
Ra, Rb, and Rc forming neither the monocyclic ring nor the fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

The “aromatic hydrocarbon ring” for the A1 ring and A2 ring has the same structure as a compound formed by introducing a hydrogen atom to the “aryl group” described above.

Ring atoms of the “aromatic hydrocarbon ring” for the A1 ring and the A2 ring include two carbon atoms on a fused bicyclic structure at the center of the formula (4).

Specific examples of the “substituted or unsubstituted aromatic hydrocarbon ring having 6 to 50 ring carbon atoms” include a compound formed by introducing a hydrogen atom to the “aryl group” described in the specific example group G1.

The “heterocycle” for the A1 ring and A2 ring has the same structure as a compound formed by introducing a hydrogen atom to the “heterocyclic group” described above.

Ring atoms of the “heterocycle” for the A1 ring and the A2 ring include two carbon atoms on a fused bicyclic structure at the center of the formula (4).

Specific examples of the “substituted or unsubstituted heterocycle having 5 to 50 ring atoms” include a compound formed by introducing a hydrogen atom to the “heterocyclic group” described in the specific example group G2.

Rb is bonded to any one of carbon atoms forming the aromatic hydrocarbon ring for the A1 ring or any one of the atoms forming the heterocycle for the A1 ring.

Rc is bonded to any one of carbon atoms forming the aromatic hydrocarbon ring for the A2 ring or any one of the atoms forming the heterocycle for the A2 ring.

At least one of Ra, Rb, or Rc is preferably a group represented by a formula (4a) below. More preferably, at least two of Ra, Rb, and Rc are each a group represented by the formula (4a).

[Formula 482]

*-L₄₀₁-Ar₄₀₁ (4)

In the formula (4a):
L₄₀₁ is a single bond, a substituted or unsubstituted arylene group having 6 to 30 ring carbon atoms, or a substituted or unsubstituted divalent heterocyclic group having 5 to 30 ring atoms; and
Ar₄₀₁ is a substituted or unsubstituted aryl group having 6 to 50 ring carbon atoms, a substituted or unsubstituted heterocyclic group having 5 to 50 ring atoms, or a group represented by a formula (4b) below.

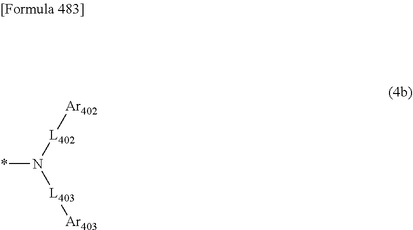

In the formula (4b):
L₄₀₂ and L₄₀₃ are each independently a single bond, a substituted or unsubstituted arylene group having 6 to 30 ring carbon atoms, or a substituted or unsubstituted divalent heterocyclic group having 5 to 30 ring atoms;
a combination of Ar₄₀₂ and Ar₄₀₃ are mutually bonded to form a substituted or unsubstituted monocyclic ring, mutually bonded to form a substituted or unsubstituted fused ring, or not mutually bonded; and
Ar₄₀₂ and Ar₄₀₃ forming neither the monocyclic ring nor the fused ring are each independently a substituted or unsubstituted aryl group having 6 to 50 ring carbon atoms, or a substituted or unsubstituted heterocyclic group having 5 to 50 ring atoms.

In an exemplary embodiment, the compound represented by the formula (4) is represented by a formula (42) below.

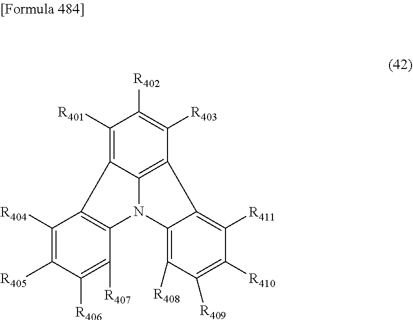

In the formula (42):
at least one combination of adjacent two or more of R₄₀₁ to R₄₁₁ are mutually bonded to form a substituted or unsubstituted monocyclic ring, mutually bonded to form a substituted or unsubstituted fused ring, or not mutually bonded; and
R₄₀₁ to R₄₁₁ forming neither the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

At least one of R₄₀₁ to R₄₁₁ is preferably a group represented by the formula (4a). More preferably, at least two of R₄₀₁ to R₄₁₁ are each a represented by the formula (4a).
R₄₀₄ and R₄₁₁ are each preferably a group represented by the formula (4a).

In an exemplary embodiment, the compound represented by the formula (4) is a compound formed by bonding a structure represented by a formula (4-1) or a formula (4-2) below to the A1 ring.

Further, in an exemplary embodiment, the compound represented by the formula (42) is a compound formed by bonding a structure represented by the formula (4-1) or the formula (4-2) to the ring bonded with R₄₀₄ to R₄₀₇.

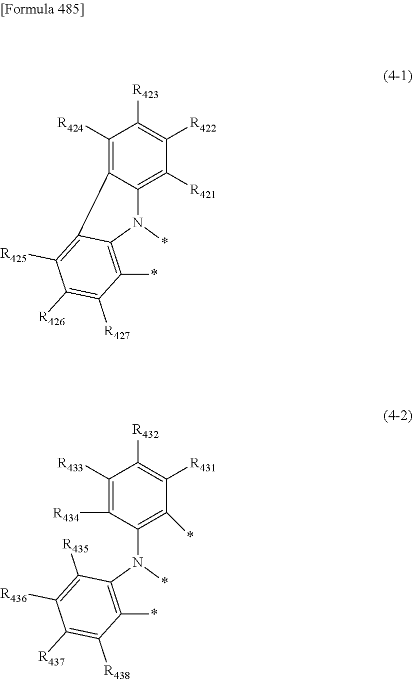

In the formula (4-1), two bonds * are each independently bonded to a ring-forming carbon atom of the aromatic hydrocarbon ring or a ring atom of the heterocycle for the A1 ring in the formula (4) or bonded to one of R₄₀₄ to R₄₀₇ in the formula (42).

In the formula (4-2), three bonds * are each independently bonded to a ring-forming carbon atom of the aromatic hydrocarbon ring or a ring atom of the heterocycle for the A1 ring in the formula (4) or bonded to one of R₄₀₄ to R₄₀₇ in the formula (42);
at least one combination of adjacent two or more of R₄₂₁ to R₄₂₇ are mutually bonded to form a substituted or unsubstituted monocyclic ring, mutually bonded to form a substituted or unsubstituted fused ring, or not mutually bonded;
at least one combination of adjacent two or more of R₄₃₁ to R₄₃₈ are mutually bonded to form a substituted or unsubstituted monocyclic ring, mutually bonded to form a substituted or unsubstituted fused ring, or not mutually bonded; and
R₄₂₁ to R₄₂₇ and R₄₃₁ to R₄₃₈ forming neither the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, the compound represented by the formula (4) is a compound represented by a formula (41-3), a formula (41-4), or a formula (41-5) below.

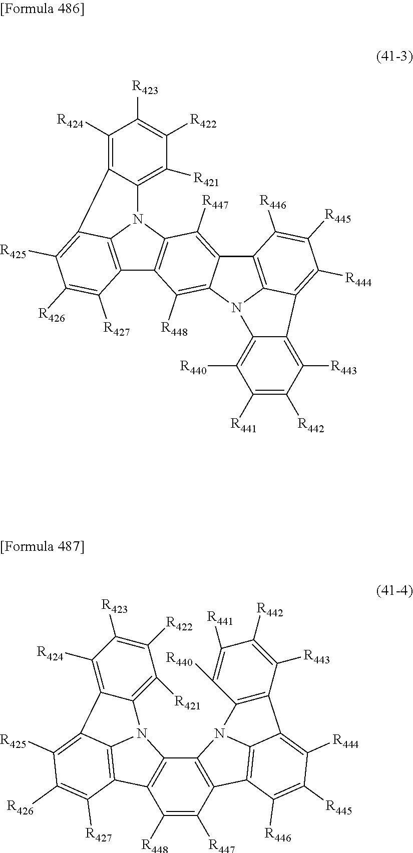

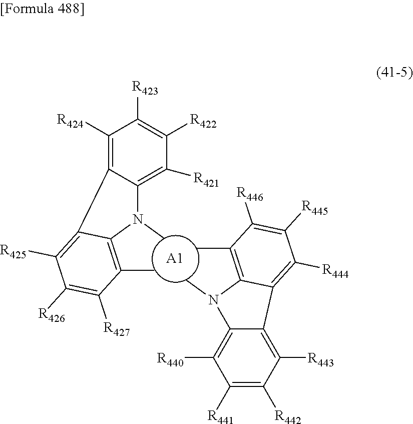

In the formulae (41-3), (41-4) and (41-5):
A1 ring is as defined for the formula (4);
R₄₂₁ to R₄₂₇ each independently represent the same as R₄₂₁ to R₄₂₇ in the formula (4-1); and
R₄₄₀ to R₄₄₈ each independently represent the same as R₄₀₁ to R₄₁₁ in the formula (42).

In an exemplary embodiment, a substituted or unsubstituted aromatic hydrocarbon ring having 6 to 50 ring carbon atoms for the A1 ring in the formula (41-5) is a substituted or unsubstituted naphthalene ring, or a substituted or unsubstituted fluorene ring.

In an exemplary embodiment, a substituted or unsubstituted heterocycle having 5 to 50 ring atoms for the A1 ring in the formula (41-5) is a substituted or unsubstituted dibenzofuran ring, a substituted or unsubstituted carbazole ring, or a substituted or unsubstituted dibenzothiophene ring.

In an exemplary embodiment, the compound represented by the formula (4) or the formula (42) is selected from the group consisting of compounds represented by formulae (461) to (467) below.

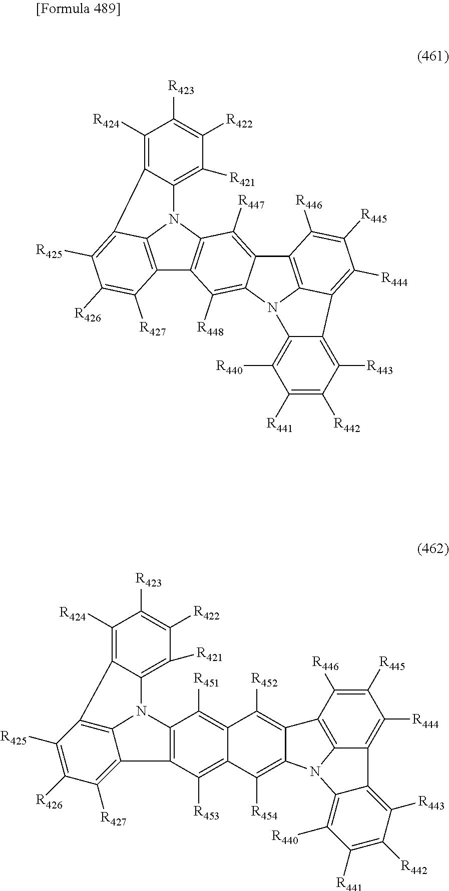

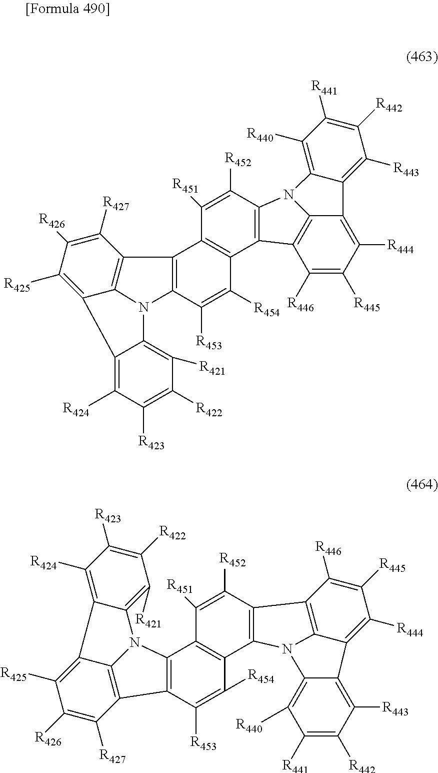

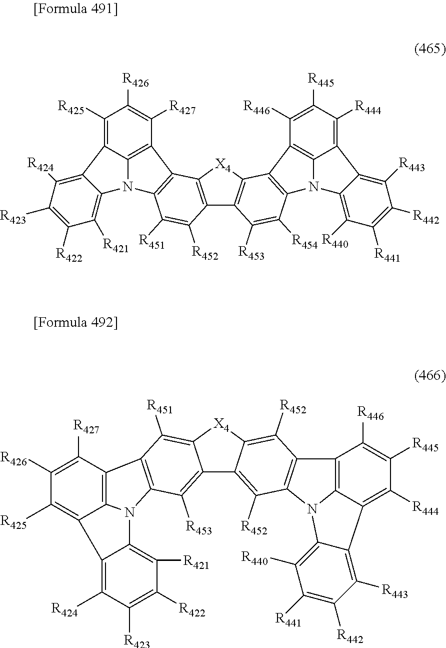

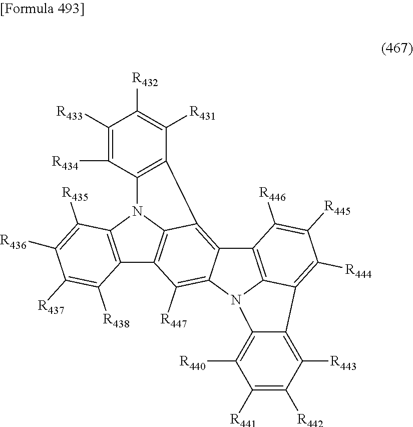

In the formulae (461), (462), (463), (464), (465), (466) and (467):
R₄₂₁ to R₄₂₇ each independently represent the same as R₄₂₁ to R₄₂₇ in the formula (4-1);
R₄₃₁ to R₄₃₈ each independently represent the same as R₄₃₁ to R₄₃₈ in the formula (4-2);
R₄₄₀ to R₄₄₈ and R₄₅₁ to R₄₅₄ each independently represent the same as R₄₀₁ to R₄₁₁ in the formula (42);
X₄ is an oxygen atom, NR₈₀₁, or C(R₈₀₂)(R₈₀₃);
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

In an exemplary embodiment, in the compound represented by the formula (42), at least one combination of adjacent two or more of R₄₀₁ to R₄₁₁ are mutually bonded to form a substituted or unsubstituted monocyclic ring or a substituted or unsubstituted fused ring. The compound represented by the formula (42) in the exemplary embodiment is described in detail as a compound represented by a formula (45).

### Compound Represented by Formula (45)

The compound represented by the formula (45) will be described.

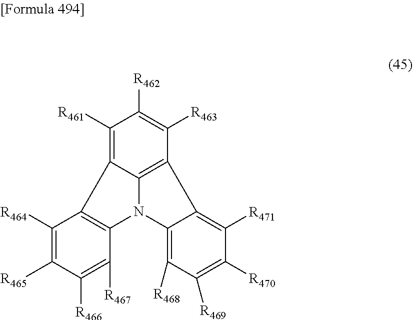

In the formula (45):
two or more of combinations selected from the group consisting of a combination of R₄₆₁ and R₄₆₂, a combination of R₄₆₂ and R₄₆₃, a combination of R₄₆₄ and R₄₆₅, a combination of R₄₆₅ and R₄₆₆, a combination of R₄₆₆ and R₄₆₇, a combination of R₄₆₈ and R₄₆₉, a combination of R₄₆₉ and R₄₇₀, and a combination of R₄₇₀ and R₄₇₁ are mutually bonded to form a substituted or unsubstituted monocyclic ring or a substituted or unsubstituted fused ring;
however, the combination of R₄₆₁ and R₄₆₂ and the combination of R₄₆₂ and R₄₆₃, the combination of R₄₆₄ and R₄₆₅ and the combination of R₄₆₅ and R₄₆₆, the combination of R₄₆₅ and R₄₆₆ and the combination of R₄₆₆ and R₄₆₇, the combination of R₄₆₈ and R₄₆₉ and the combination of R₄₆₉ and R₄₇₀, and the combination of R₄₆₉ and R₄₇₀ and the combination of R₄₇₀ and R₄₇₁ do not form a ring at the same time;
at least two rings formed by R₄₆₁ to R₄₇₁ are mutually the same or different; and
R₄₆₁ to R₄₇₁ forming neither the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (45), Rₙ and Rₙ₊₁ (n being an integer selected from 461, 462, 464 to 466, and 468 to 470) are mutually bonded to form a substituted or unsubstituted monocyclic ring or fused ring together with two ring-forming carbon atoms bonded with Rₙ and Rₙ₊₁. The ring is preferably formed of atoms selected from the group consisting of a carbon atom, an oxygen atom, a sulfur atom, and a nitrogen atom, and is preferably made of 3 to 7, more preferably 5 or 6 atoms.

The number of the above cyclic structures in the compound represented by the formula (45) is, for instance, 2, 3, or 4. The two or more of the cyclic structures may be present on the same benzene ring on the basic skeleton represented by the formula (45) or may be present on different benzene rings. For instance, when three cyclic structures are present, each of the cyclic structures may be present on corresponding one of the three benzene rings of the formula (45).

Examples of the above cyclic structures in the compound represented by the formula (45) include structures represented by formulae (451) to (460) below.

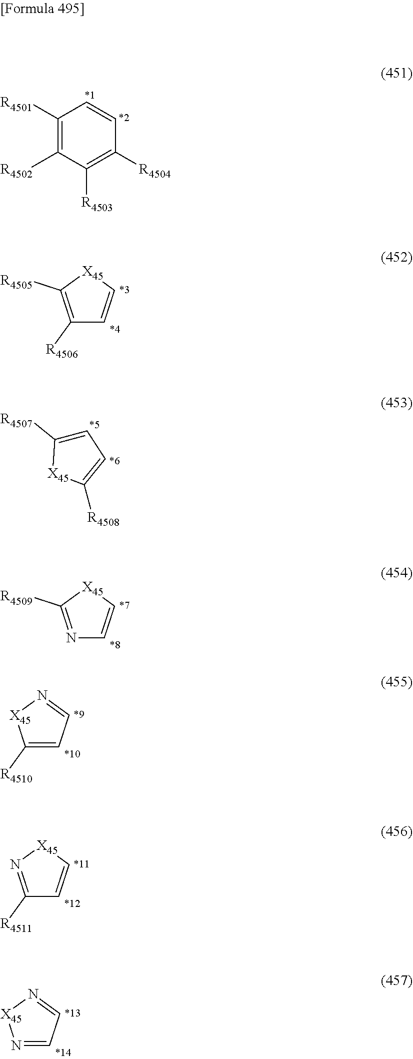

In the formulae (451) to (457):
each combination of *1 and *2, *3 and *4, *5 and *6, *7 and *8, *9 and *10, *11 and *12, and *13 and *14 represent the two ring-forming carbon atoms bonded with Rₙ and Rₙ₊₁;
the ring-forming carbon atom bonded with Rₙ may be any one of the two ring-forming carbon atoms represented by *1 and *2, *3 and *4, *5 and *6, *7 and *8, *9 and *10, *11 and *12, and *13 and *14;
X₄₅ is C(R₄₅₁₂)(R₄₅₁₃), NR₄₅₁₄, an oxygen atom, or a sulfur atom;
at least one combination of adjacent two or more of R₄₅₀₁ to R₄₅₀₆ and R₄₅₁₂ to R₄₅₁₃ are mutually bonded to form a substituted or unsubstituted monocyclic ring, mutually bonded to form a substituted or unsubstituted fused ring, or not mutually bonded; and
R₄₅₀₁ to R₄₅₁₄ forming neither the monocyclic ring nor the fused ring each independently represent the same as R₄₆₁ to R₄₇₁ in the formula (45).

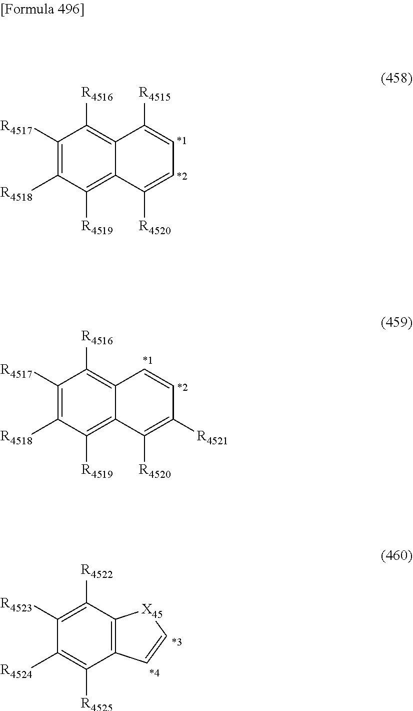

In the formulae (458) to (460):
each combination of *1 and *2, and *3 and *4 represent the two ring-forming carbon atoms bonded with Rₙ and Rₙ₊₁;
the ring-forming carbon atom bonded with Rₙ may be any one of the two ring-forming carbon atoms represented by *1 and *2, or *3 and *4;
X₄₅ is C(R₄₅₁₂)(R₄₅₁₃), NR₄₅₁₄, an oxygen atom, or a sulfur atom;
at least one combination of adjacent two or more of R₄₅₁₂ to R₄₅₁₃ and R₄₅₁₅ to R₄₅₂₅ are mutually bonded to form a substituted or unsubstituted monocyclic ring, mutually bonded to form a substituted or unsubstituted fused ring, or not mutually bonded; and
R₄₅₁₂ to R₄₅₁₃, R₄₅₁₅ to R₄₅₂₁ and R₄₅₂₂ to R₄₅₂₅ forming neither the monocyclic ring nor the fused ring, and R₄₅₁₄ each independently represent the same as R₄₆₁ to R₄₇₁ in the formula (45).

In the formula (45), it is preferable that at least one of R₄₆₂, R₄₆₄, R₄₆₅, R₄₇₀ or R₄₇₁ (preferably, at least one of R₄₆₂, R₄₆₅ or R₄₇₀, more preferably R₄₆₂) is a group forming no cyclic structure.
(i) A substituent, if present, for a cyclic structure formed by Rₙ and Rₙ₊₁ in the formula (45),
(ii) R₄₆₁ to R₄₇₁ forming no cyclic structure in the formula (45), and (iii) R₄₅₀₁ to R₄₅₁₄, R₄₅₁₅ to R₄₅₂₅ in the formulae (451) to (460) are preferably each independently any one of groups selected from the group consisting of a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), a substituted or unsubstituted aryl group having 6 to 50 ring carbon atoms, a substituted or unsubstituted heterocyclic group having 5 to 50 ring atoms, or groups represented by formulae (461) to (464).

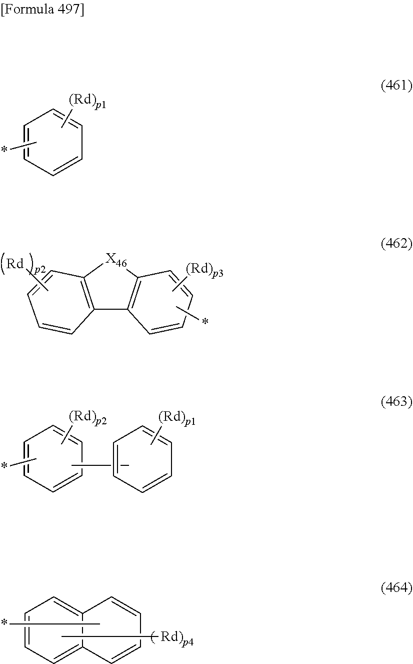

In the formulae (461) to (464):
R_{d} are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
X₄₆ is C(R₈₀₁)(R₈₀₂), NR₈₀₃, an oxygen atom or a sulfur atom;
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different;
when a plurality of R₈₀₃ are present, the plurality of R₈₀₃ are mutually the same or different;
p1 is 5;
p2 is 4;
p3 is 3;
p4 is 7; and
* in the formulae (461) to (464) each independently represent a bonding position to a cyclic structure.

In the third and fourth compounds, R₉₀₁ to R₉₀₇ represent the same as those as described above.

In an exemplary embodiment, the compound represented by the formula (45) is represented by one of formulae (45-1) to (45-6) below.

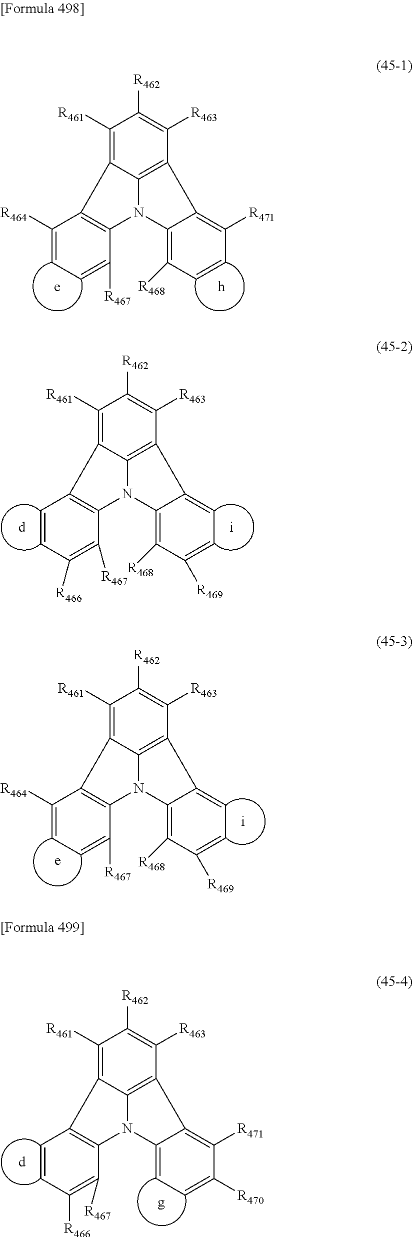

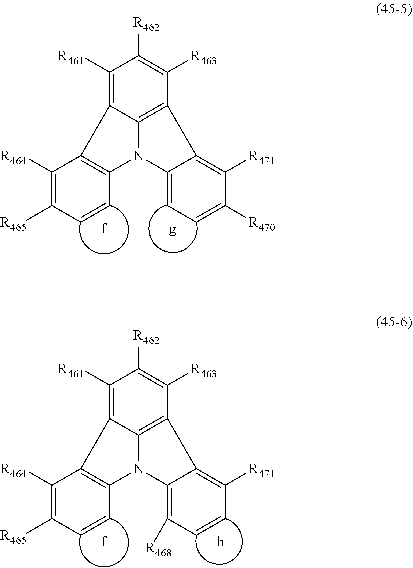

In the formulae (45-1) to (45-6):
rings d to i are each independently a substituted or unsubstituted monocyclic ring or a substituted or unsubstituted fused ring; and
R₄₆₁ to R₄₇₁ each independently represent the same as R₄₆₁ to R₄₇₁ in the formula (45).

In an exemplary embodiment, the compound represented by the formula (45) is represented by one of formulae (45-7) to (45-12) below.

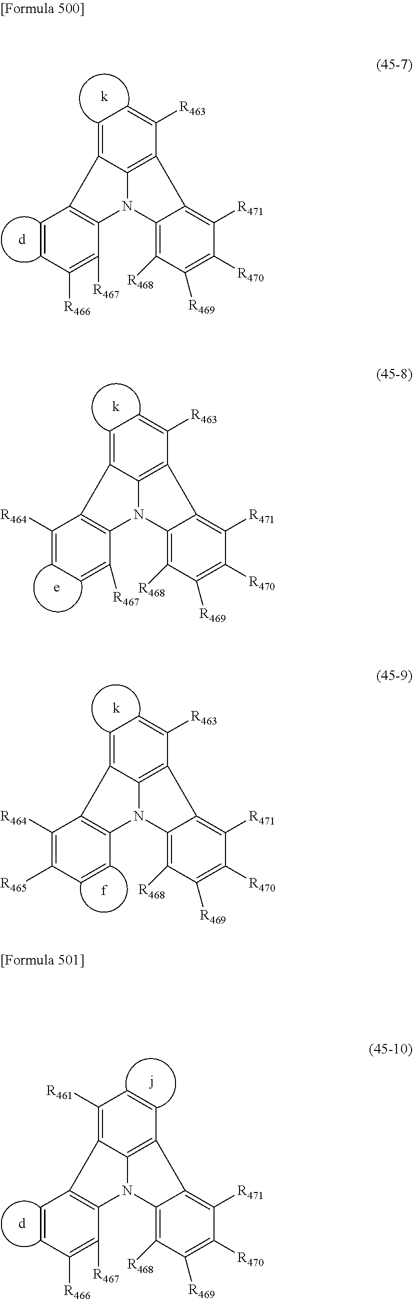

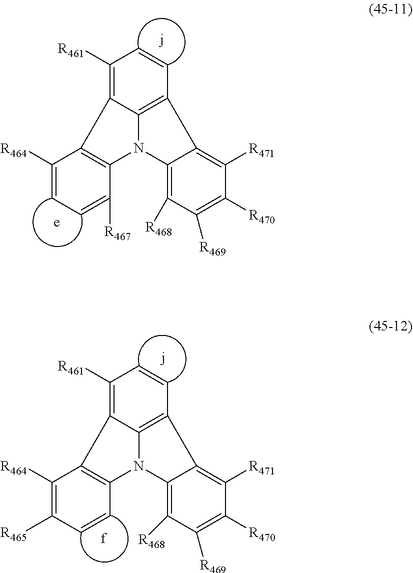

In the formulae (45-7) to (45-12):
rings d to f, k and j are each independently a substituted or unsubstituted monocyclic ring or a substituted or unsubstituted fused ring; and
R₄₆₁ to R₄₇₁ each independently represent the same as R₄₆₁ to R₄₇₁ in the formula (45).

In an exemplary embodiment, the compound represented by the formula (45) is represented by one of formulae (45-13) to (45-21) below.

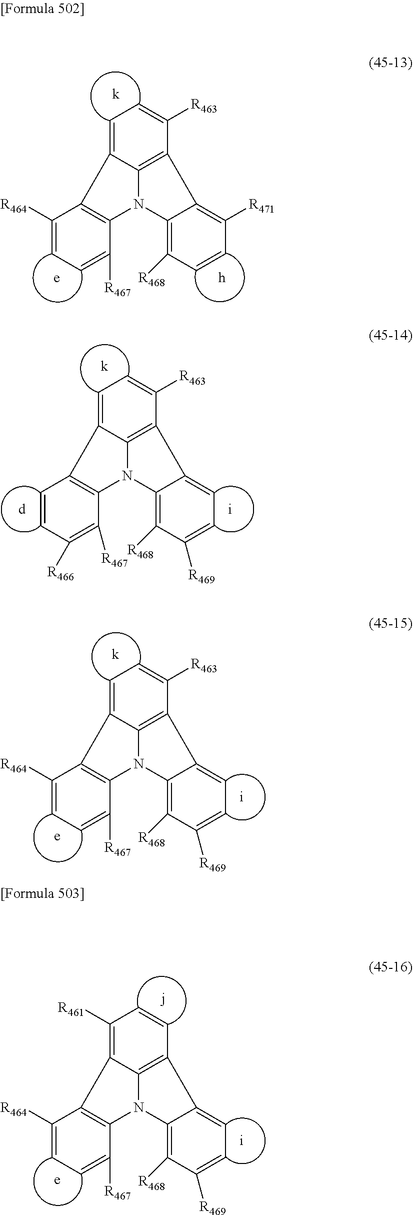

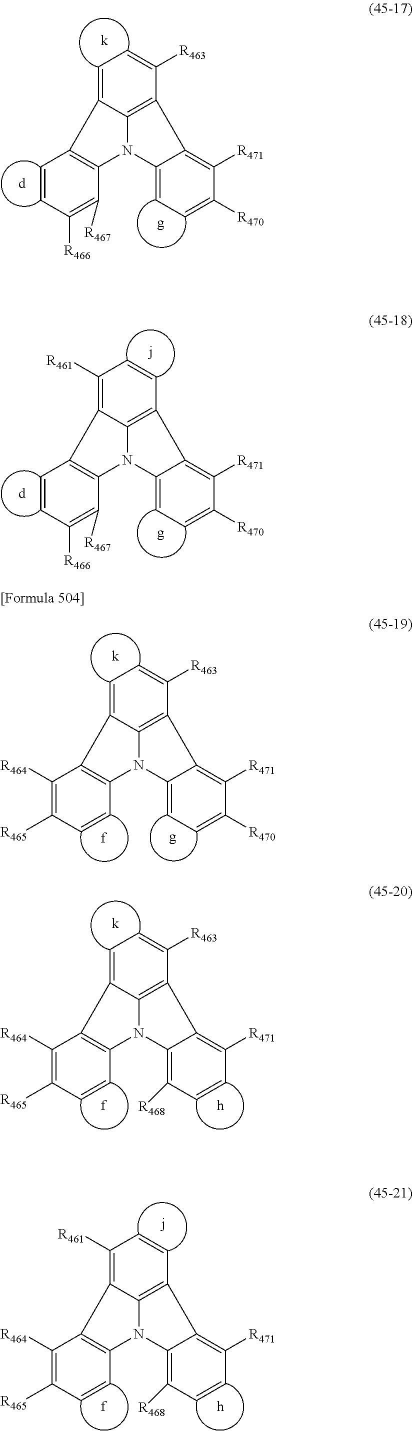

In the formulae (45-13) to (45-21):
rings d to k are each independently a substituted or unsubstituted monocyclic ring or a substituted or unsubstituted fused ring; and
R₄₆₁ to R₄₇₁ each independently represent the same as R₄₆₁ to R₄₇₁ in the formula (45).

When the ring g or the ring h further has a substituent, examples of the substituent include a substituted or unsubstituted alkyl group having 1 to 50 carbon atoms, a substituted or unsubstituted aryl group having 6 to 50 ring carbon atoms, a group represented by the formula (461), a group represented by the formula (463), and a group represented by the formula (464).

In an exemplary embodiment, the compound represented by the formula (45) is represented by one of formulae (45-22) to (45-25) below.

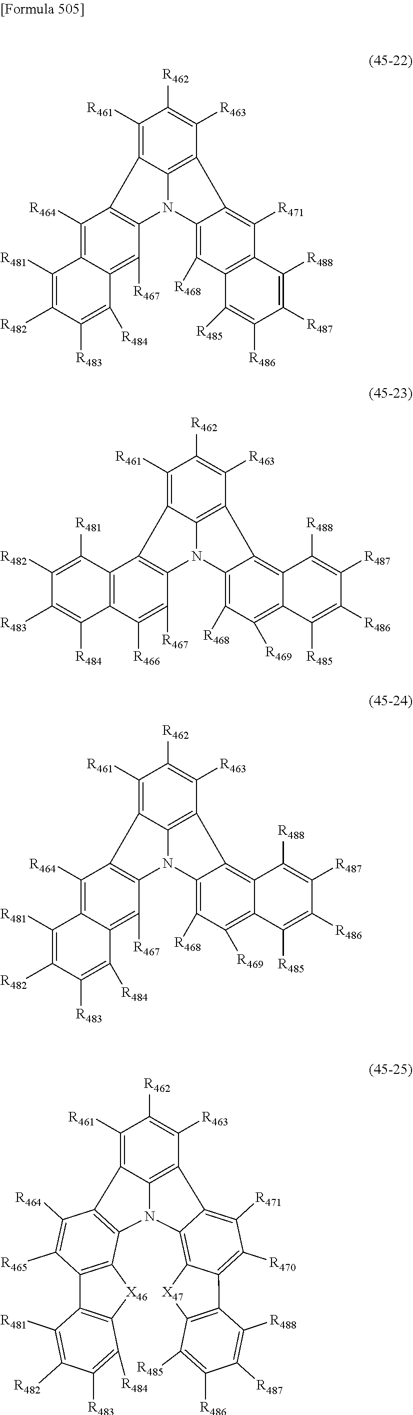

In the formulae (45-22) to (45-25):
X₄₆ and X₄₇ are each independently C(R₈₀₁)(R₈₀₂), NR₈₀₃, an oxygen atom or a sulfur atom;
R₄₆₁ to R₄₇₁ and R₄₈₁ to R₄₈₈ each independently represent the same as R₄₆₁ to R₄₇₁ of the formula (45);
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

In an exemplary embodiment, the compound represented by the formula (45) is represented by a formula (45-26) below.

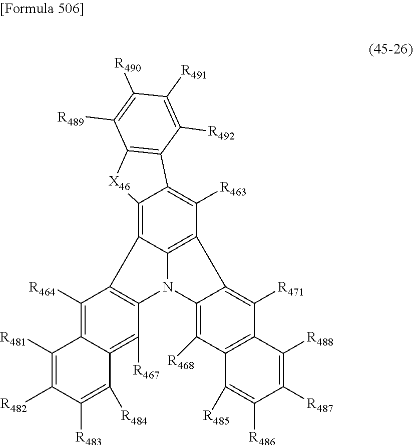

In the formula (45-26):
X₄₆ is C(R₈₀₁)(R₈₀₂), NR₈₀₃, an oxygen atom or a sulfur atom;
R₄₆₃, R₄₆₄, R₄₆₇, R₄₆₈, R₄₇₁, and R₄₈₁ to R₄₉₂ each independently represent the same as R₄₆₁ to R₄₇₁ in the formula (45);
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

Specific examples of the compound represented by the formula (4) include compounds shown below. In the specific examples below, Ph represents a phenyl group, and D represents a deuterium atom.

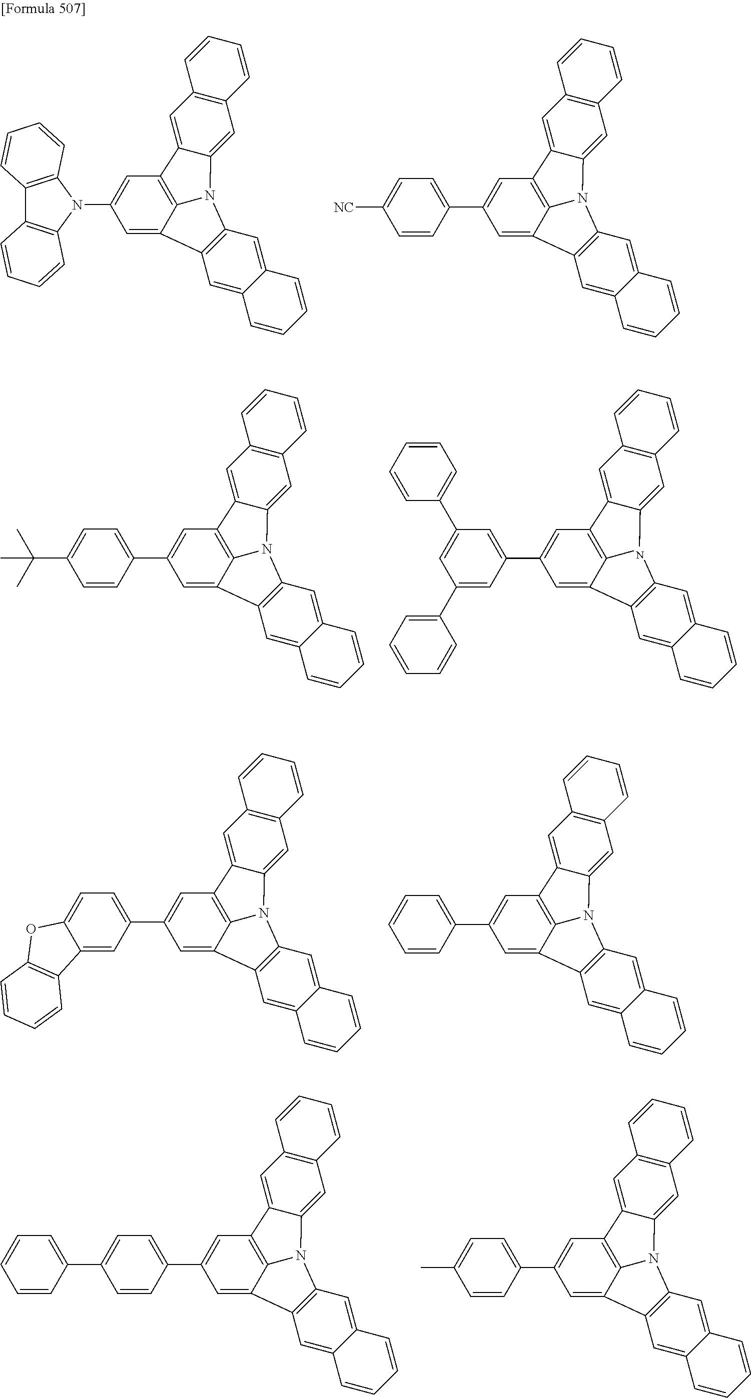

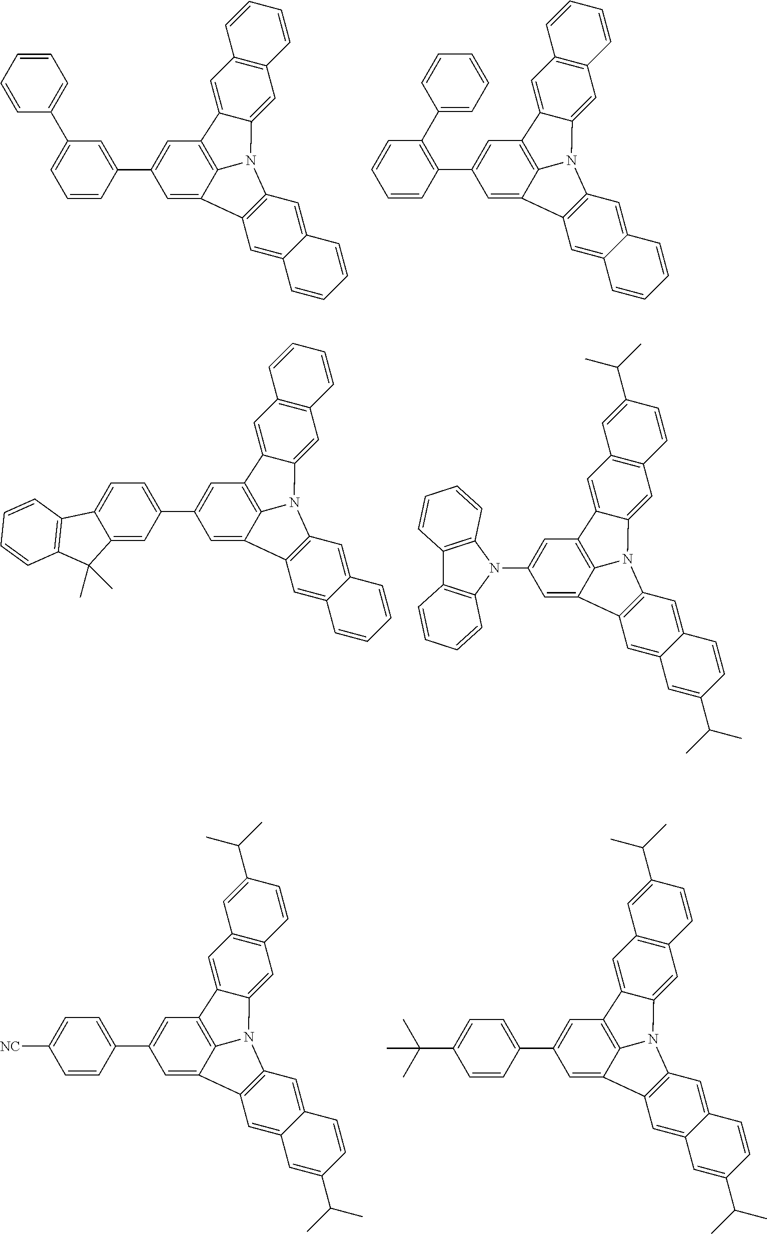

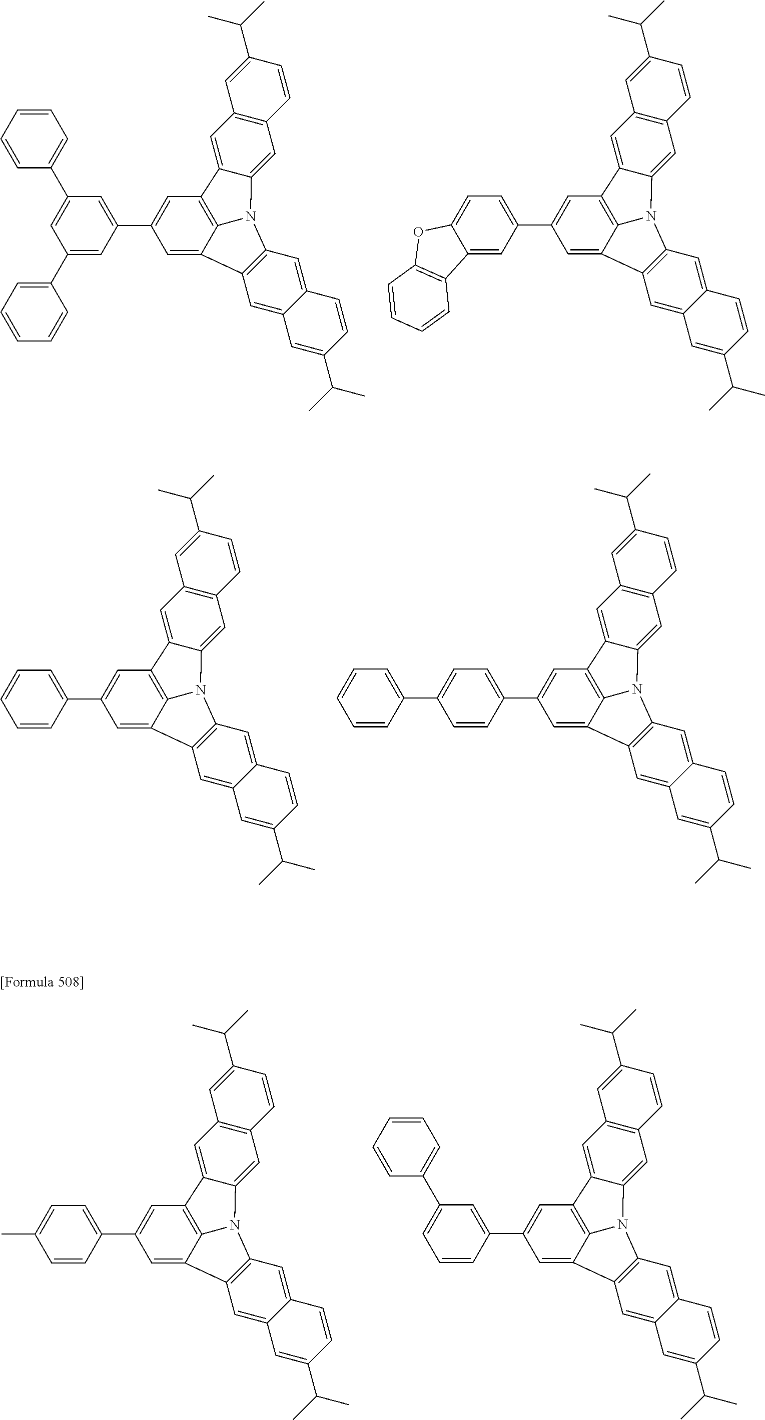

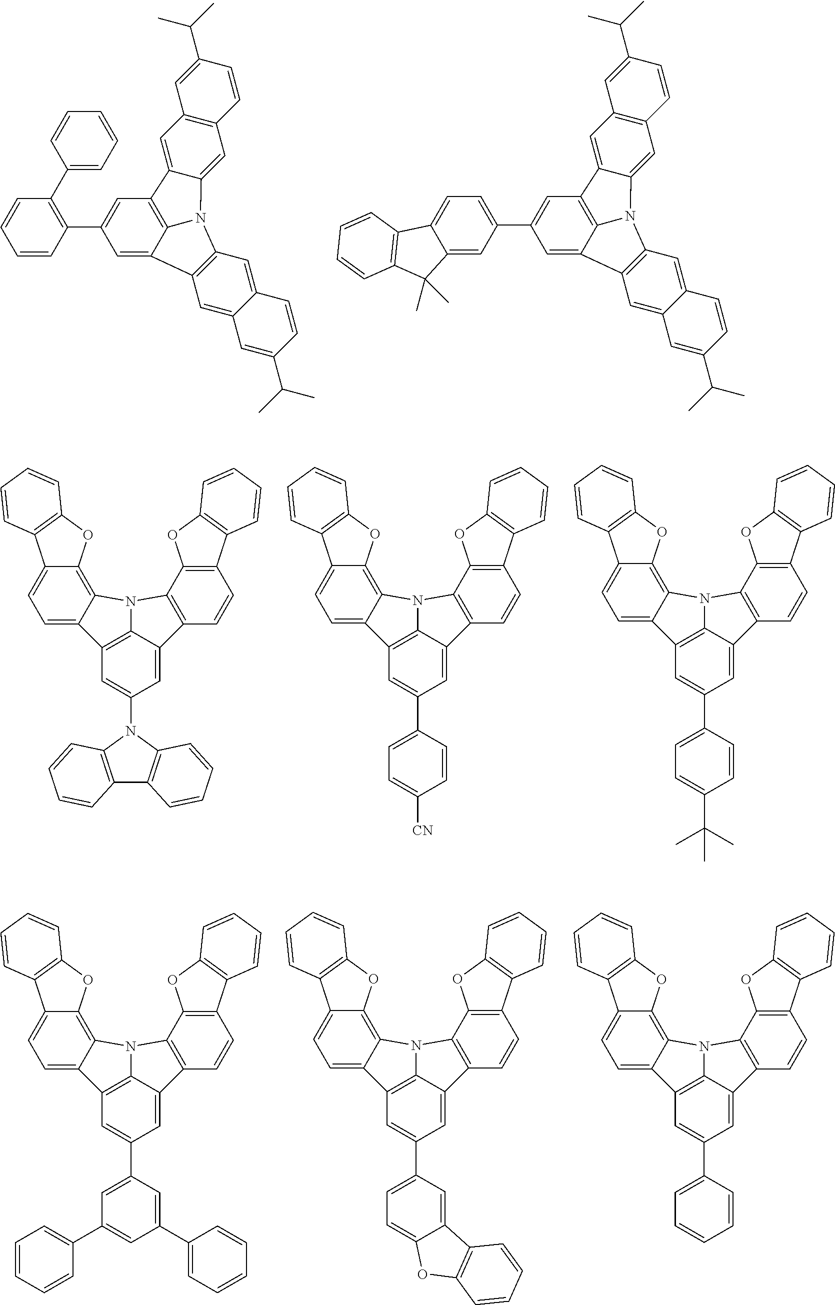

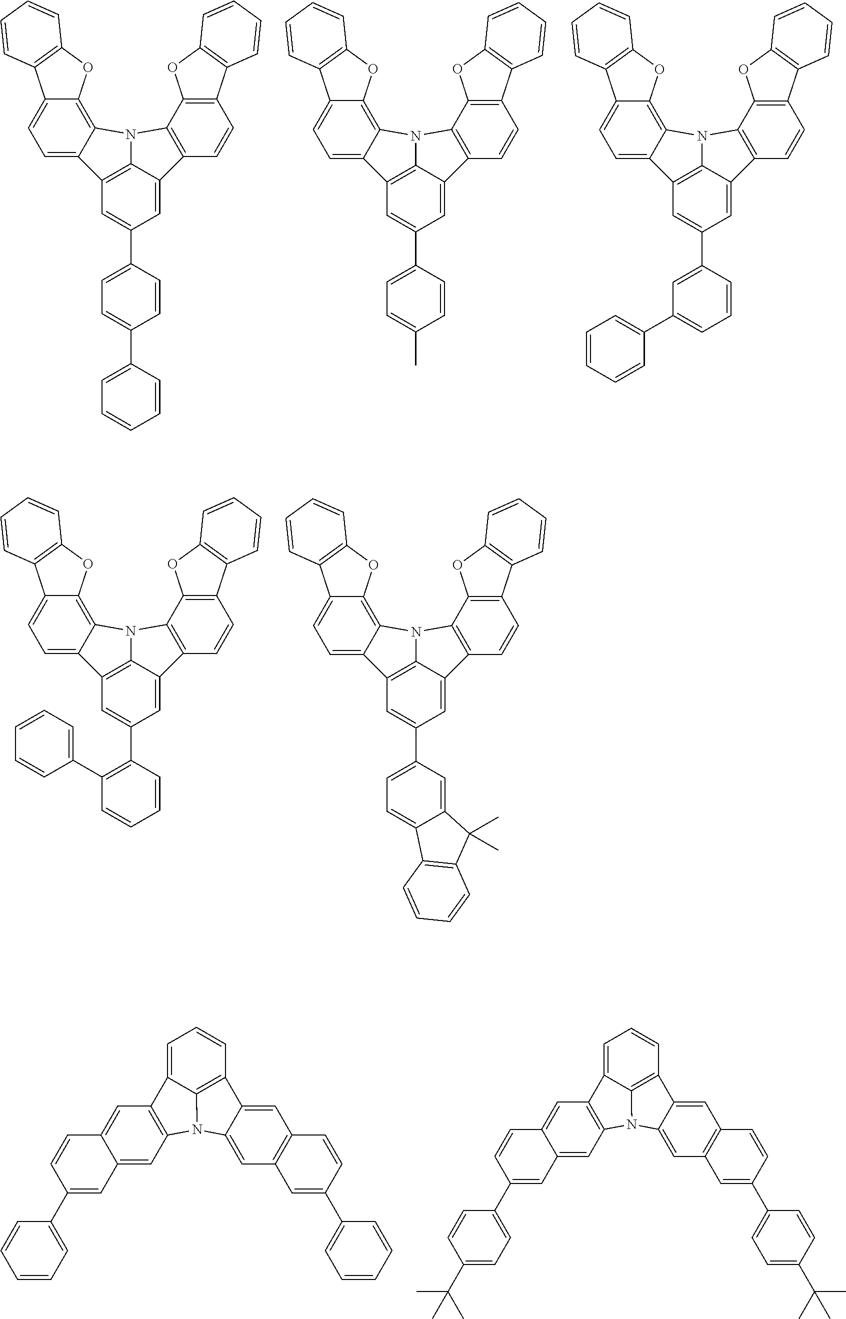

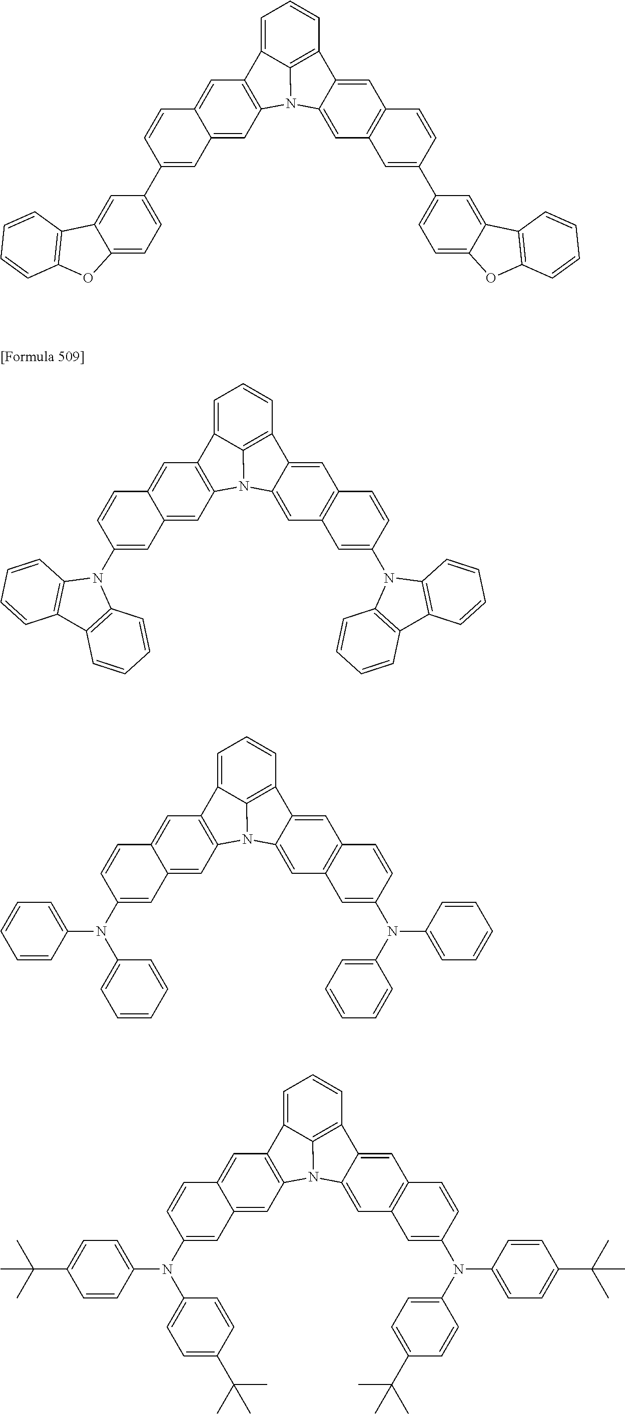 [SMILES: c1ccc2c(c1)oc1ccc(-c3ccc4cc5c6cccc7c8cc9ccc(-c%10ccc%11oc%12ccccc%12c%11c%10)cc9cc8n(c5cc4c3)c67)cc12 ; c1ccc2c(c1)c1ccccc1n2-c1ccc2cc3c4cccc5c6cc7ccc(-n8c9ccccc9c9ccccc98)cc7cc6n(c3cc2c1)c45 ; c1ccc(N(c2ccccc2)c2ccc3cc4c5cccc6c7cc8ccc(N(c9ccccc9)c9ccccc9)cc8cc7n(c4cc3c2)c56)cc1 ; CC(C)(C)c1ccc(N(c2ccc(C(C)(C)C)cc2)c2ccc3cc4c5cccc6c7cc8ccc(N(c9ccc(C(C)(C)C)cc9)c9ccc(C(C)(C)C)cc9)cc8cc7n(c4cc3c2)c56)cc1]

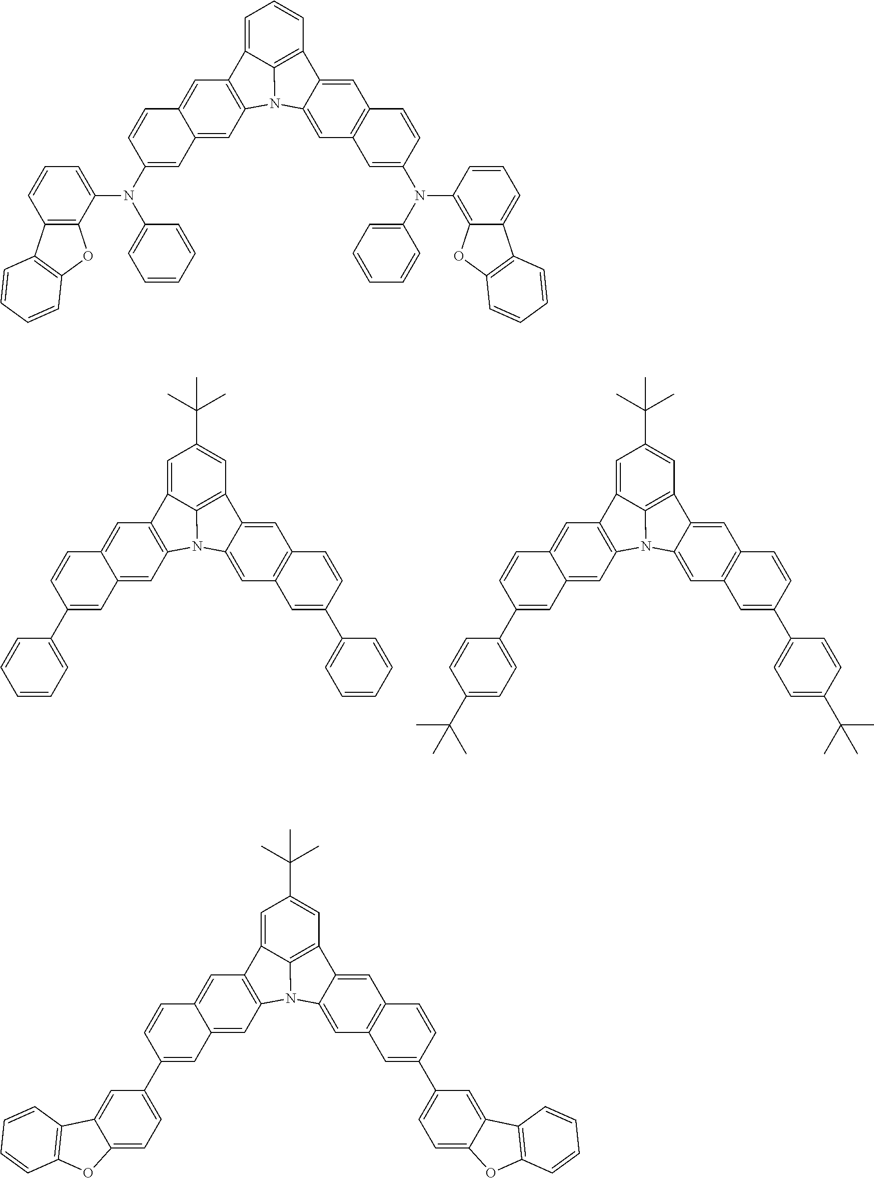 [SMILES: c1ccc(N(c2ccc3cc4c5cccc6c7cc8ccc(N(c9ccccc9)c9cccc%10c9oc9ccccc9%10)cc8cc7n(c4cc3c2)c56)c2cccc3c2oc2ccccc23)cc1 ; CC(C)(C)c1cc2c3cc4ccc(-c5ccccc5)cc4cc3n3c4cc5cc(-c6ccccc6)ccc5cc4c(c1)c23 ; CC(C)(C)c1ccc(-c2ccc3cc4c5cc(C(C)(C)C)cc6c7cc8ccc(-c9ccc(C(C)(C)C)cc9)cc8cc7n(c4cc3c2)c56)cc1 ; CC(C)(C)c1cc2c3cc4ccc(-c5ccc6oc7ccccc7c6c5)cc4cc3n3c4cc5cc(-c6ccc7oc8ccccc8c7c6)ccc5cc4c(c1)c23]

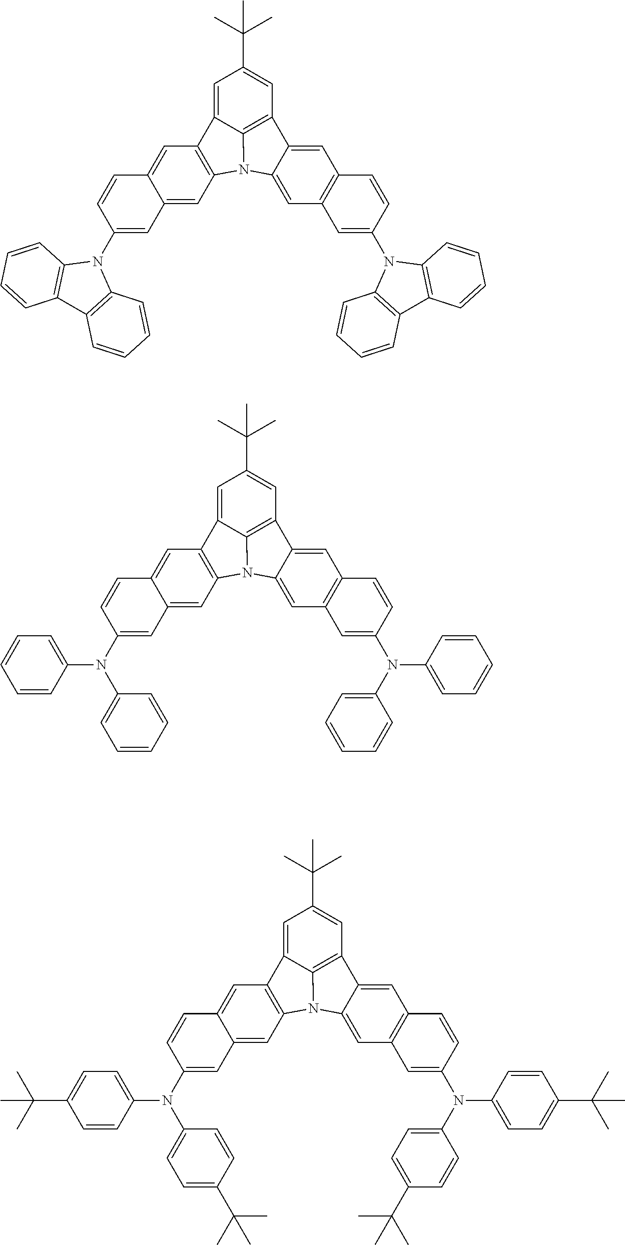 [SMILES: CC(C)(C)c1cc2c3cc4ccc(-n5c6ccccc6c6ccccc65)cc4cc3n3c4cc5cc(-n6c7ccccc7c7ccccc76)ccc5cc4c(c1)c23 ; CC(C)(C)c1cc2c3cc4ccc(N(c5ccccc5)c5ccccc5)cc4cc3n3c4cc5cc(N(c6ccccc6)c6ccccc6)ccc5cc4c(c1)c23 ; CC(C)(C)c1ccc(N(c2ccc(C(C)(C)C)cc2)c2ccc3cc4c5cc(C(C)(C)C)cc6c7cc8ccc(N(c9ccc(C(C)(C)C)cc9)c9ccc(C(C)(C)C)cc9)cc8cc7n(c4cc3c2)c56)cc1]

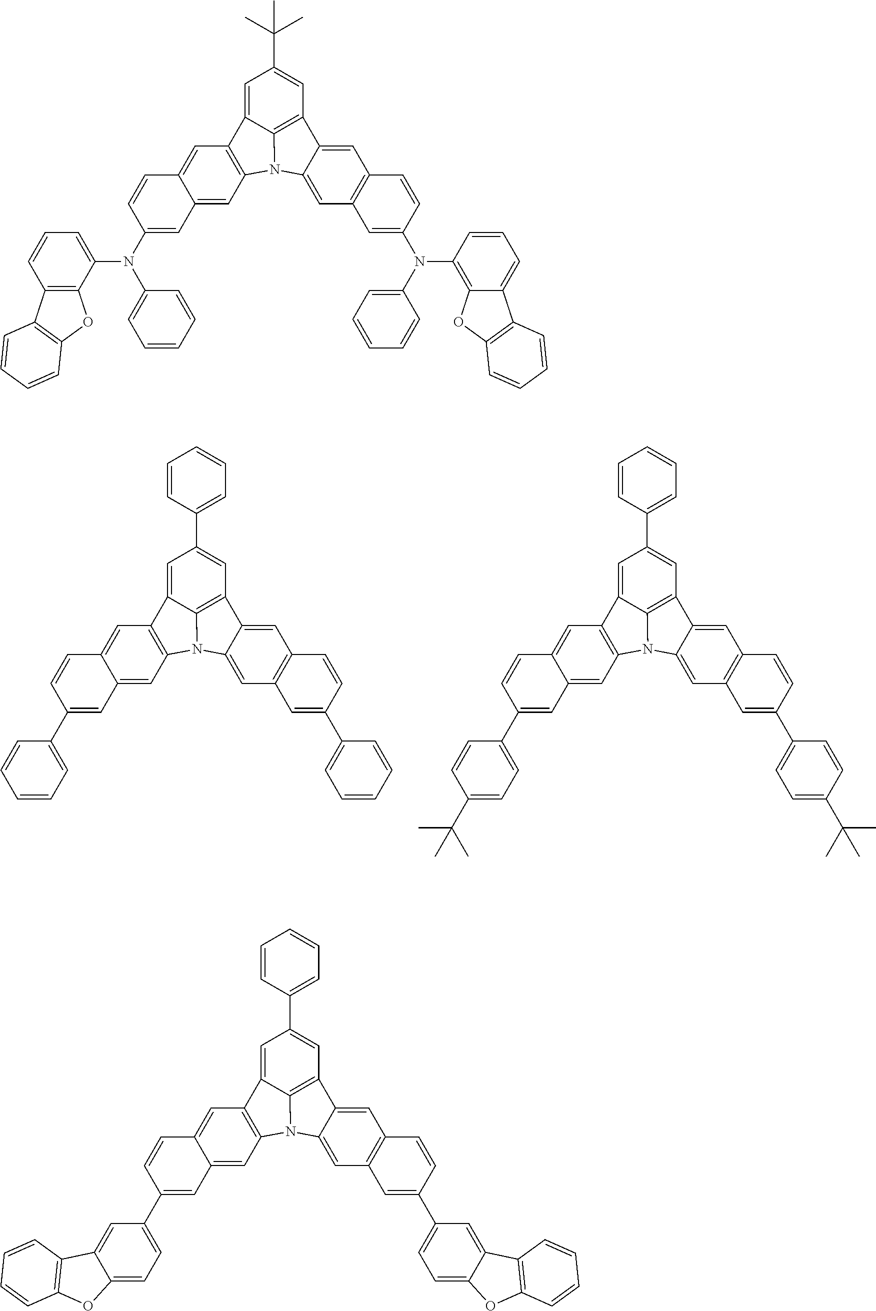 [SMILES: CC(C)(C)c1cc2c3cc4ccc(N(c5ccccc5)c5cccc6c5oc5ccccc56)cc4cc3n3c4cc5cc(N(c6ccccc6)c6cccc7c6oc6ccccc67)ccc5cc4c(c1)c23 ; c1ccc(-c2ccc3cc4c5cc(-c6ccccc6)cc6c7cc8ccc(-c9ccccc9)cc8cc7n(c4cc3c2)c56)cc1 ; CC(C)(C)c1ccc(-c2ccc3cc4c5cc(-c6ccccc6)cc6c7cc8ccc(-c9ccc(C(C)(C)C)cc9)cc8cc7n(c4cc3c2)c56)cc1 ; c1ccc(-c2cc3c4cc5ccc(-c6ccc7oc8ccccc8c7c6)cc5cc4n4c5cc6cc(-c7ccc8oc9ccccc9c8c7)ccc6cc5c(c2)c34)cc1]

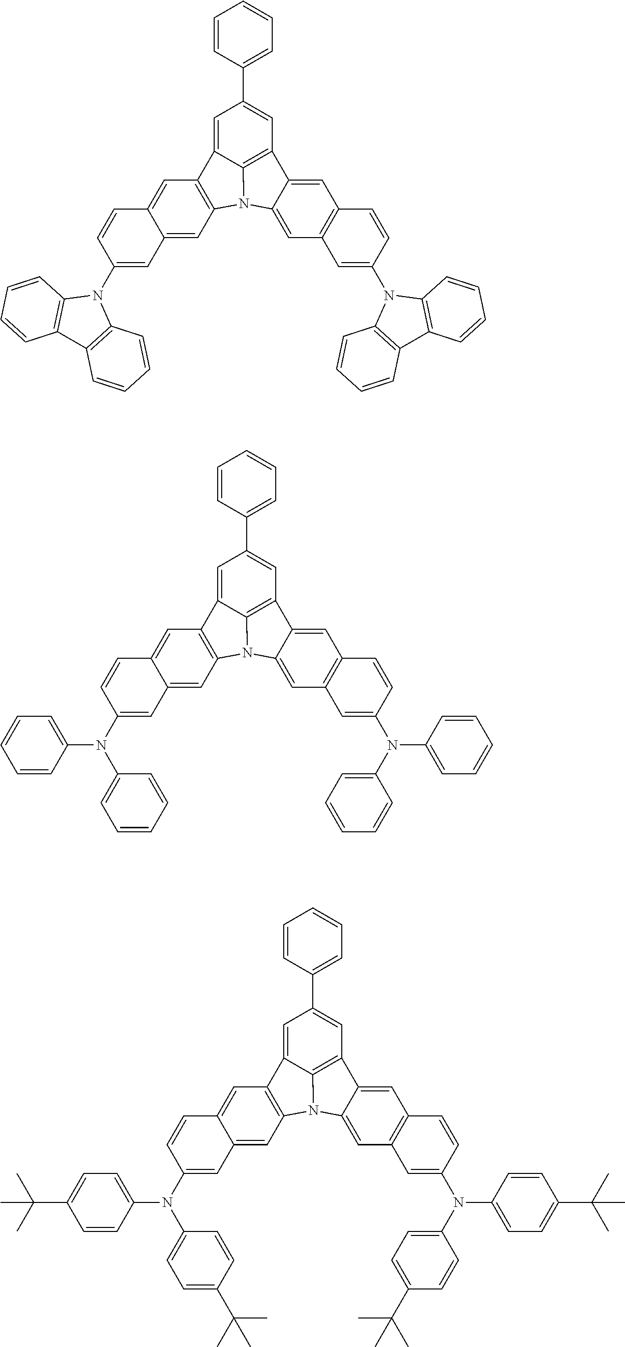 [SMILES: c1ccc(-c2cc3c4cc5ccc(-n6c7ccccc7c7ccccc76)cc5cc4n4c5cc6cc(-n7c8ccccc8c8ccccc87)ccc6cc5c(c2)c34)cc1 ; c1ccc(-c2cc3c4cc5ccc(N(c6ccccc6)c6ccccc6)cc5cc4n4c5cc6cc(N(c7ccccc7)c7ccccc7)ccc6cc5c(c2)c34)cc1 ; CC(C)(C)c1ccc(N(c2ccc(C(C)(C)C)cc2)c2ccc3cc4c5cc(-c6ccccc6)cc6c7cc8ccc(N(c9ccc(C(C)(C)C)cc9)c9ccc(C(C)(C)C)cc9)cc8cc7n(c4cc3c2)c56)cc1]

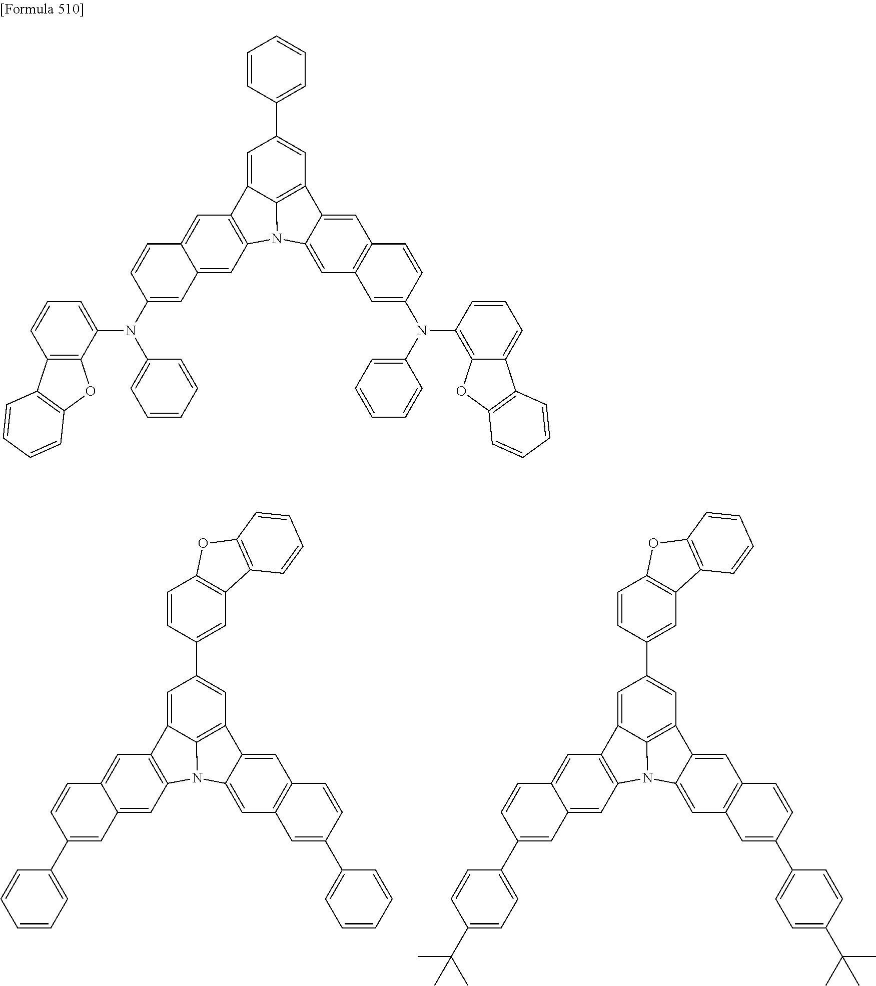 [SMILES: c1ccc(-c2cc3c4cc5ccc(N(c6ccccc6)c6cccc7c6oc6ccccc67)cc5cc4n4c5cc6cc(N(c7ccccc7)c7cccc8c7oc7ccccc78)ccc6cc5c(c2)c34)cc1 ; c1ccc(-c2ccc3cc4c5cc(-c6ccc7oc8ccccc8c7c6)cc6c7cc8ccc(-c9ccccc9)cc8cc7n(c4cc3c2)c56)cc1 ; CC(C)(C)c1ccc(-c2ccc3cc4c5cc(-c6ccc7oc8ccccc8c7c6)cc6c7cc8ccc(-c9ccc(C(C)(C)C)cc9)cc8cc7n(c4cc3c2)c56)cc1]

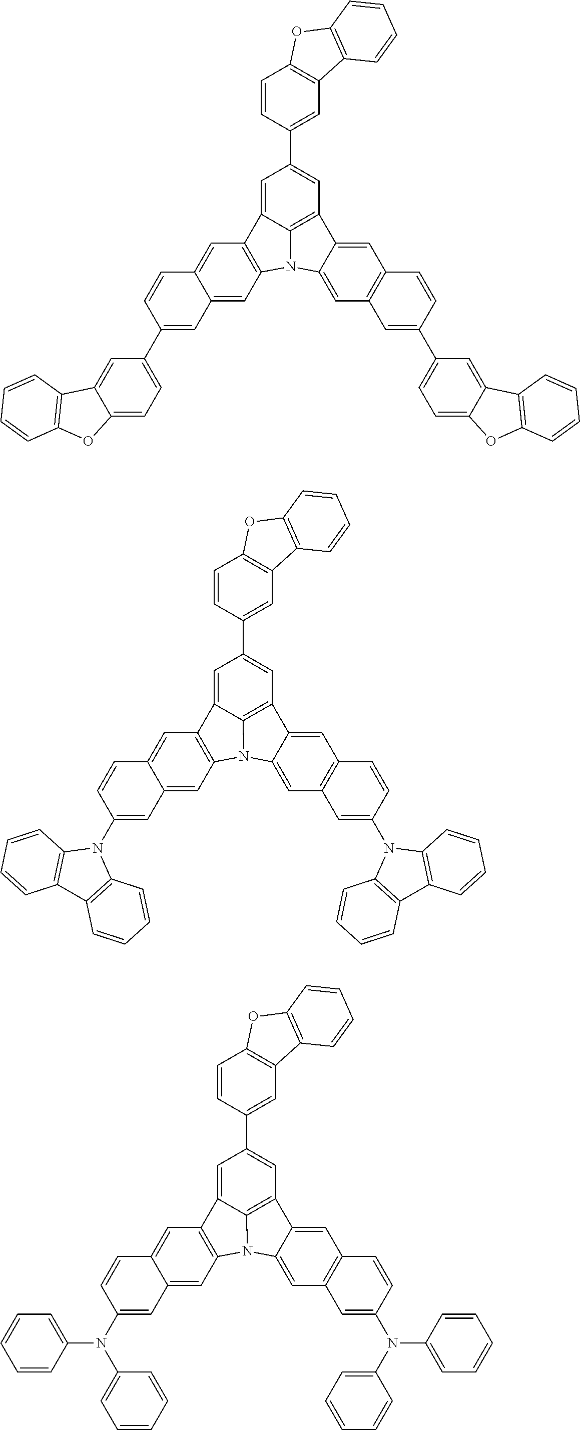 [SMILES: c1ccc2c(c1)oc1ccc(-c3ccc4cc5c6cc(-c7ccc8oc9ccccc9c8c7)cc7c8cc9ccc(-c%10ccc%11oc%12ccccc%12c%11c%10)cc9cc8n(c5cc4c3)c67)cc12 ; c1ccc2c(c1)oc1ccc(-c3cc4c5cc6ccc(-n7c8ccccc8c8ccccc87)cc6cc5n5c6cc7cc(-n8c9ccccc9c9ccccc98)ccc7cc6c(c3)c45)cc12 ; c1ccc(N(c2ccccc2)c2ccc3cc4c5cc(-c6ccc7oc8ccccc8c7c6)cc6c7cc8ccc(N(c9ccccc9)c9ccccc9)cc8cc7n(c4cc3c2)c56)cc1]

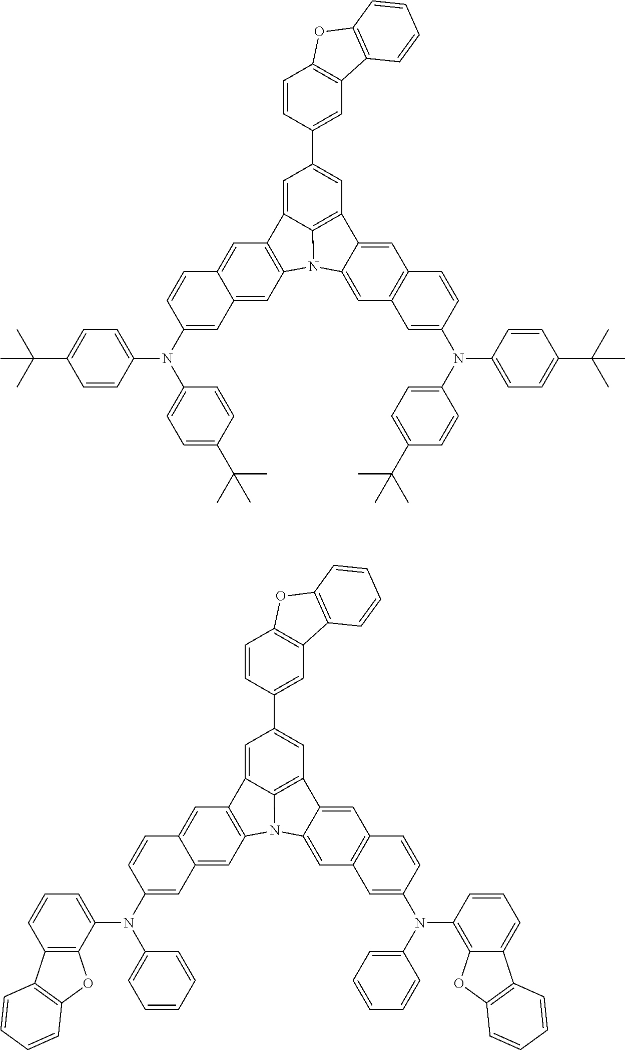 [SMILES: CC(C)(C)c1ccc(N(c2ccc(C(C)(C)C)cc2)c2ccc3cc4c5cc(-c6ccc7oc8ccccc8c7c6)cc6c7cc8ccc(N(c9ccc(C(C)(C)C)cc9)c9ccc(C(C)(C)C)cc9)cc8cc7n(c4cc3c2)c56)cc1 ; c1ccc(N(c2ccc3cc4c5cc(-c6ccc7oc8ccccc8c7c6)cc6c7cc8ccc(N(c9ccccc9)c9cccc%10c9oc9ccccc9%10)cc8cc7n(c4cc3c2)c56)c2cccc3c2oc2ccccc23)cc1]

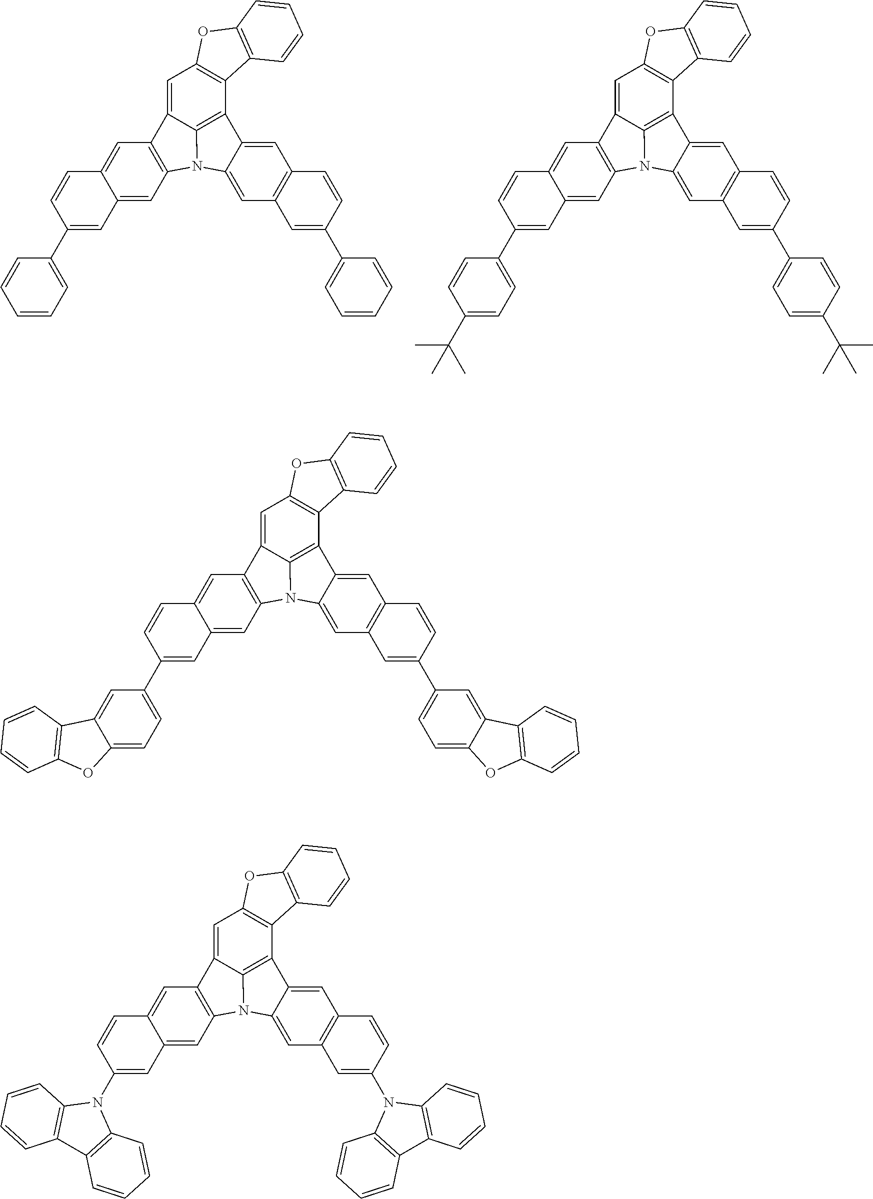 [SMILES: c1ccc(-c2ccc3cc4c5cc6oc7ccccc7c6c6c7cc8ccc(-c9ccccc9)cc8cc7n(c4cc3c2)c56)cc1 ; CC(C)(C)c1ccc(-c2ccc3cc4c5cc6oc7ccccc7c6c6c7cc8ccc(-c9ccc(C(C)(C)C)cc9)cc8cc7n(c4cc3c2)c56)cc1 ; c1ccc2c(c1)oc1ccc(-c3ccc4cc5c6cc7oc8ccccc8c7c7c8cc9ccc(-c%10ccc%11oc%12ccccc%12c%11c%10)cc9cc8n(c5cc4c3)c67)cc12 ; c1ccc2c(c1)oc1cc3c4cc5ccc(-n6c7ccccc7c7ccccc76)cc5cc4n4c5cc6cc(-n7c8ccccc8c8ccccc87)ccc6cc5c(c12)c34]

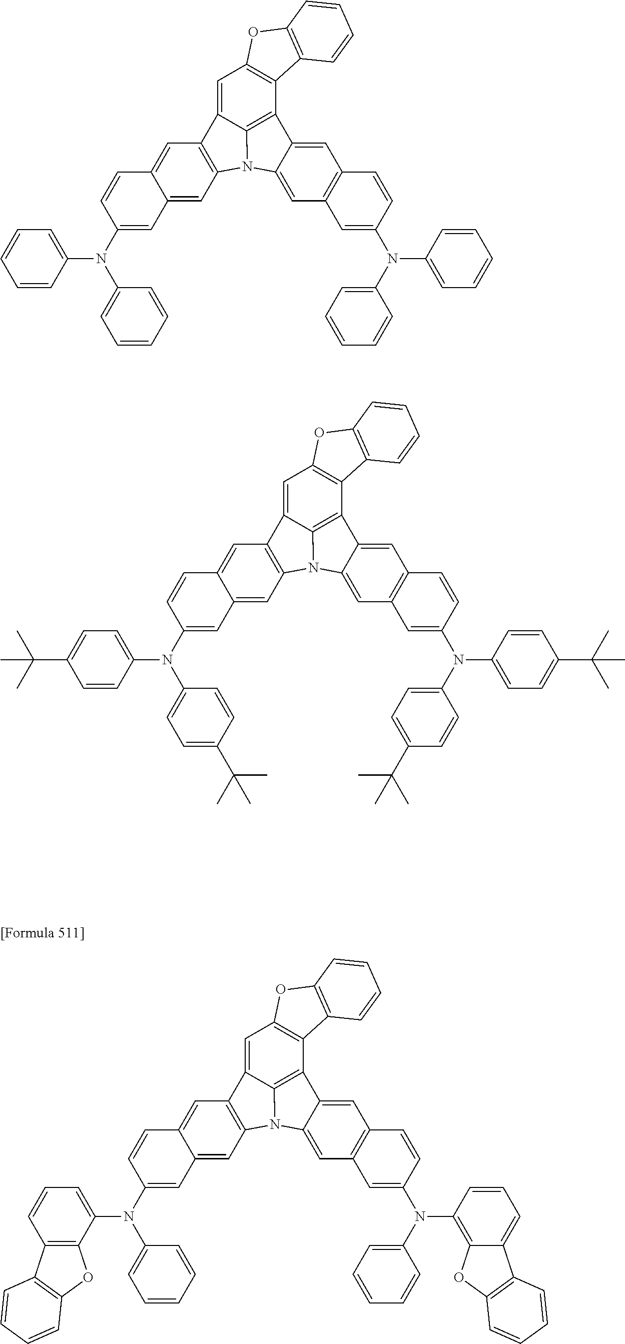 [SMILES: c1ccc(N(c2ccccc2)c2ccc3cc4c5cc6oc7ccccc7c6c6c7cc8ccc(N(c9ccccc9)c9ccccc9)cc8cc7n(c4cc3c2)c56)cc1 ; CC(C)(C)c1ccc(N(c2ccc(C(C)(C)C)cc2)c2ccc3cc4c5cc6oc7ccccc7c6c6c7cc8ccc(N(c9ccc(C(C)(C)C)cc9)c9ccc(C(C)(C)C)cc9)cc8cc7n(c4cc3c2)c56)cc1 ; c1ccc(N(c2ccc3cc4c5cc6oc7ccccc7c6c6c7cc8ccc(N(c9ccccc9)c9cccc%10c9oc9ccccc9%10)cc8cc7n(c4cc3c2)c56)c2cccc3c2oc2ccccc23)cc1]

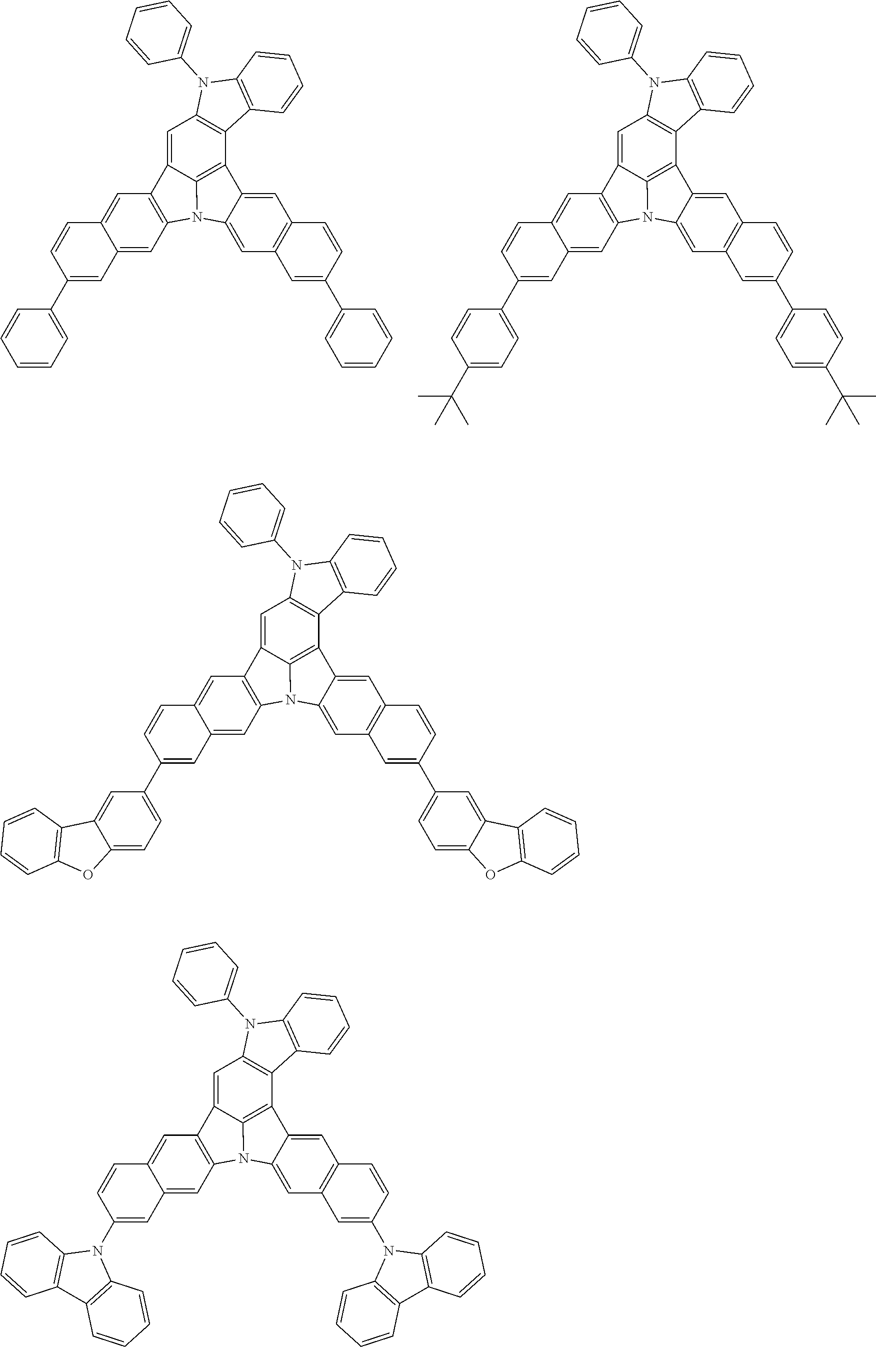 [SMILES: c1ccc(-c2ccc3cc4c5cc6c(c7ccccc7n6-c6ccccc6)c6c7cc8ccc(-c9ccccc9)cc8cc7n(c4cc3c2)c56)cc1 ; CC(C)(C)c1ccc(-c2ccc3cc4c5cc6c(c7ccccc7n6-c6ccccc6)c6c7cc8ccc(-c9ccc(C(C)(C)C)cc9)cc8cc7n(c4cc3c2)c56)cc1 ; c1ccc(-n2c3ccccc3c3c4c5cc6ccc(-c7ccc8oc9ccccc9c8c7)cc6cc5n5c6cc7cc(-c8ccc9oc%10ccccc%10c9c8)ccc7cc6c(cc32)c45)cc1 ; c1ccc(-n2c3ccccc3c3c4c5cc6ccc(-n7c8ccccc8c8ccccc87)cc6cc5n5c6cc7cc(-n8c9ccccc9c9ccccc98)ccc7cc6c(cc32)c45)cc1]

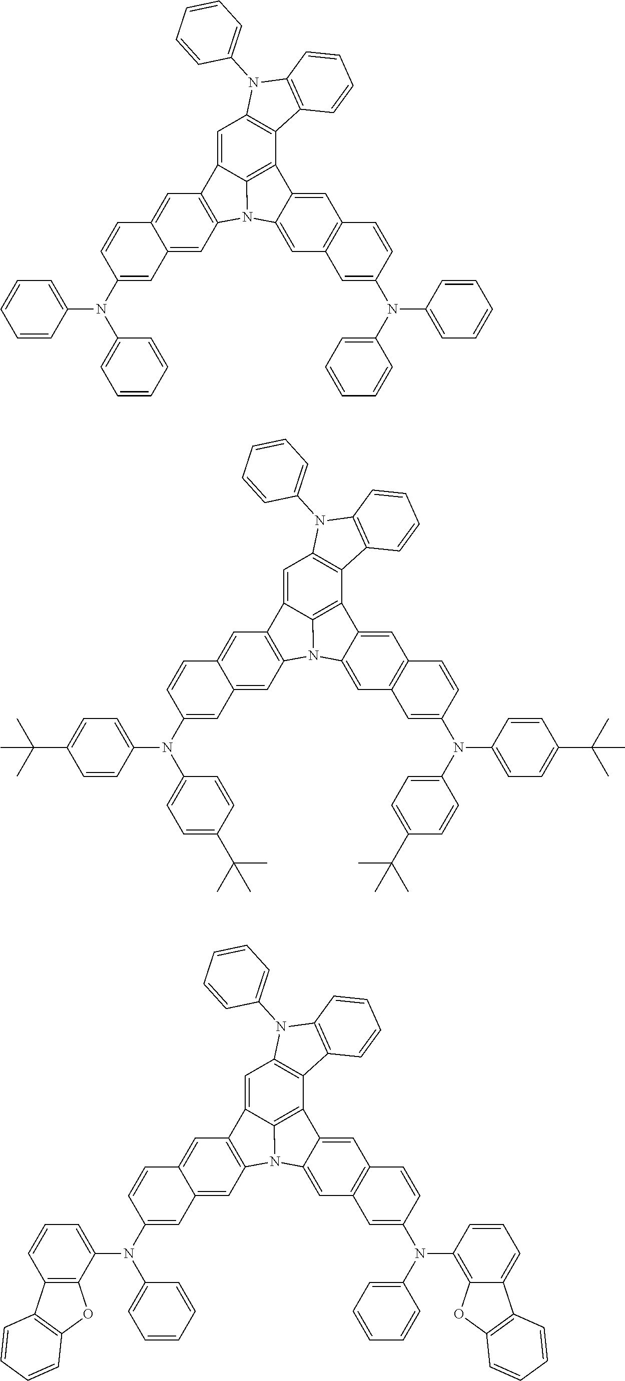 [SMILES: c1ccc(N(c2ccccc2)c2ccc3cc4c5cc6c(c7ccccc7n6-c6ccccc6)c6c7cc8ccc(N(c9ccccc9)c9ccccc9)cc8cc7n(c4cc3c2)c56)cc1 ; CC(C)(C)c1ccc(N(c2ccc(C(C)(C)C)cc2)c2ccc3cc4c5cc6c(c7ccccc7n6-c6ccccc6)c6c7cc8ccc(N(c9ccc(C(C)(C)C)cc9)c9ccc(C(C)(C)C)cc9)cc8cc7n(c4cc3c2)c56)cc1 ; c1ccc(N(c2ccc3cc4c5cc6c(c7ccccc7n6-c6ccccc6)c6c7cc8ccc(N(c9ccccc9)c9cccc%10c9oc9ccccc9%10)cc8cc7n(c4cc3c2)c56)c2cccc3c2oc2ccccc23)cc1]

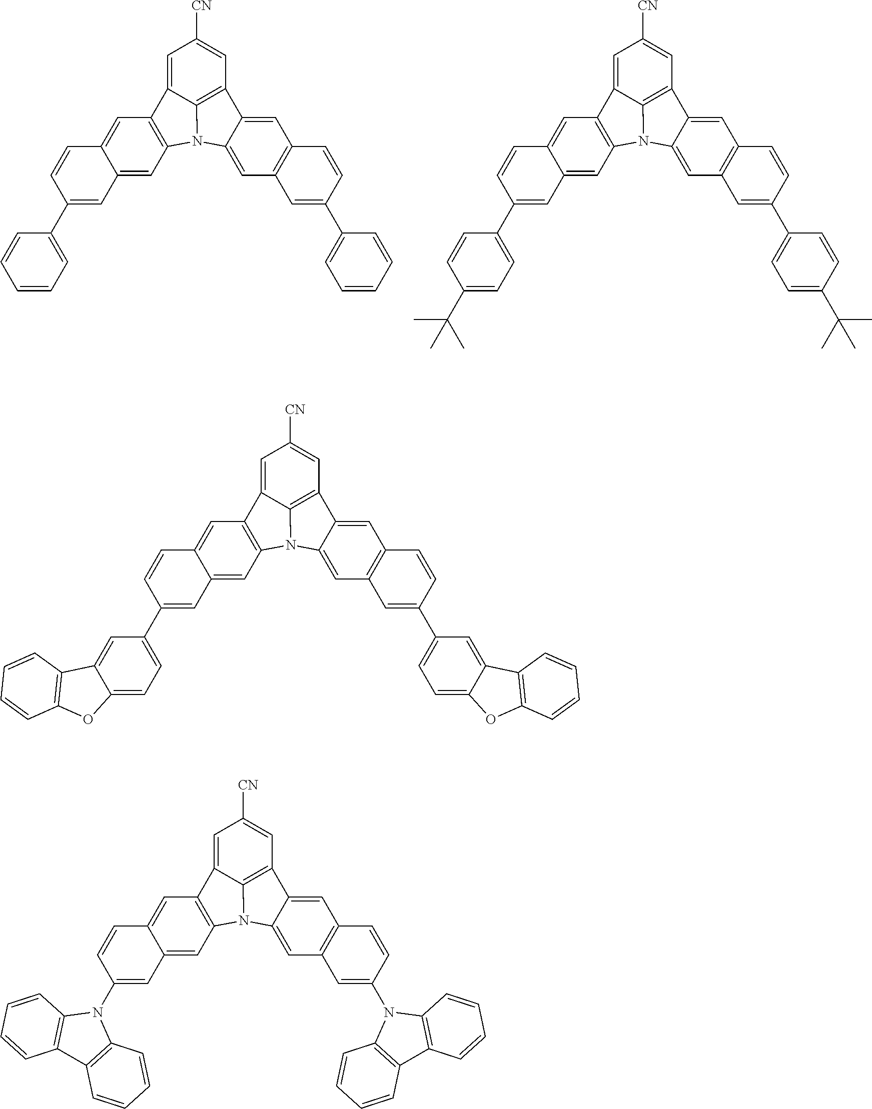 [SMILES: N#Cc1cc2c3cc4ccc(-c5ccccc5)cc4cc3n3c4cc5cc(-c6ccccc6)ccc5cc4c(c1)c23 ; CC(C)(C)c1ccc(-c2ccc3cc4c5cc(C#N)cc6c7cc8ccc(-c9ccc(C(C)(C)C)cc9)cc8cc7n(c4cc3c2)c56)cc1 ; N#Cc1cc2c3cc4ccc(-c5ccc6oc7ccccc7c6c5)cc4cc3n3c4cc5cc(-c6ccc7oc8ccccc8c7c6)ccc5cc4c(c1)c23 ; N#Cc1cc2c3cc4ccc(-n5c6ccccc6c6ccccc65)cc4cc3n3c4cc5cc(-n6c7ccccc7c7ccccc76)ccc5cc4c(c1)c23]

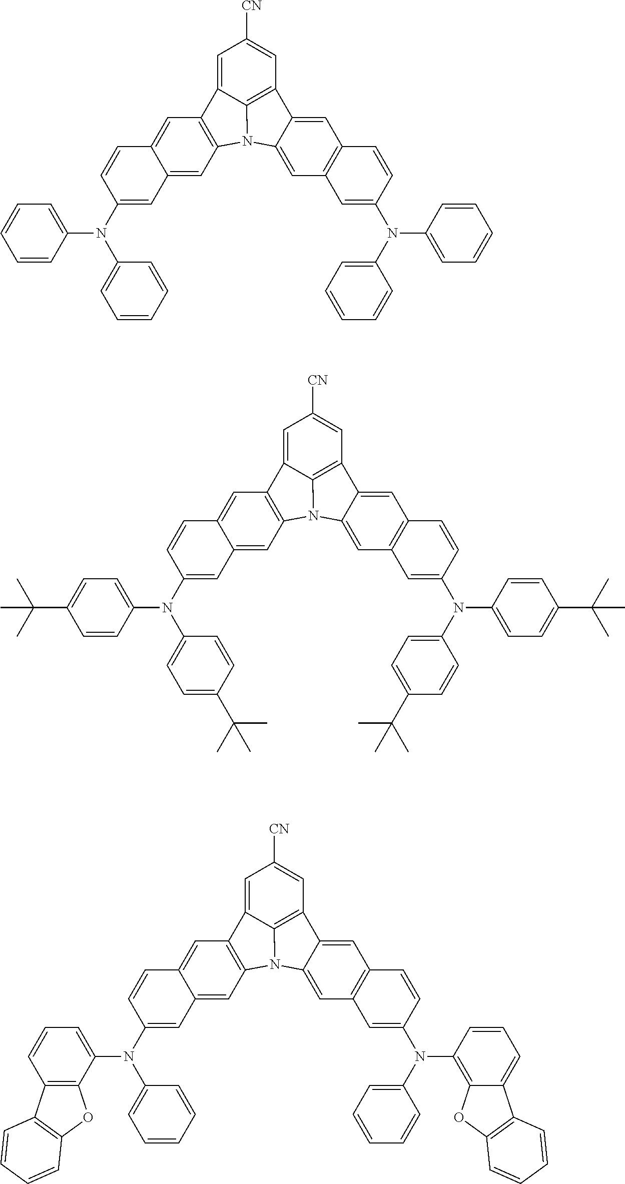 [SMILES: N#Cc1cc2c3cc4ccc(N(c5ccccc5)c5ccccc5)cc4cc3n3c4cc5cc(N(c6ccccc6)c6ccccc6)ccc5cc4c(c1)c23 ; CC(C)(C)c1ccc(N(c2ccc(C(C)(C)C)cc2)c2ccc3cc4c5cc(C#N)cc6c7cc8ccc(N(c9ccc(C(C)(C)C)cc9)c9ccc(C(C)(C)C)cc9)cc8cc7n(c4cc3c2)c56)cc1 ; N#Cc1cc2c3cc4ccc(N(c5ccccc5)c5cccc6c5oc5ccccc56)cc4cc3n3c4cc5cc(N(c6ccccc6)c6cccc7c6oc6ccccc67)ccc5cc4c(c1)c23]

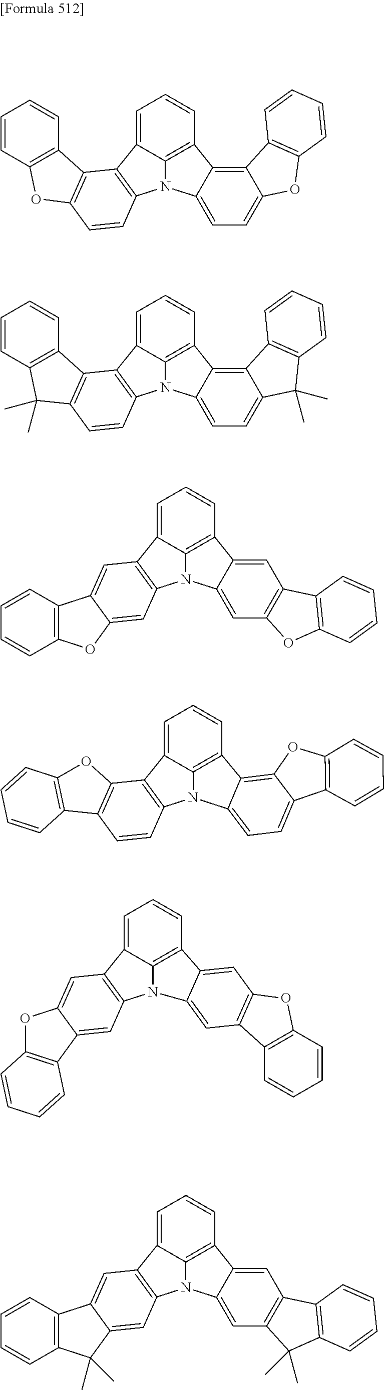 [SMILES: c1ccc2c(c1)oc1ccc3c(c4cccc5c6c7c(ccc6n3c45)oc3ccccc37)c12 ; CC1(C)c2ccccc2-c2c1ccc1c2c2cccc3c4c5c(ccc4n1c23)C(C)(C)c1ccccc1-5 ; c1ccc2c(c1)oc1cc3c(cc12)c1cccc2c4cc5c(cc4n3c12)oc1ccccc15 ; c1ccc2c(c1)oc1c2ccc2c1c1cccc3c4c5oc6ccccc6c5ccc4n2c13 ; c1ccc2c(c1)oc1cc3c4cccc5c6cc7oc8ccccc8c7cc6n(c3cc12)c45 ; CC1(C)c2ccccc2-c2cc3c4cccc5c6cc7c(cc6n(c3cc21)c45)C(C)(C)c1ccccc1-7]

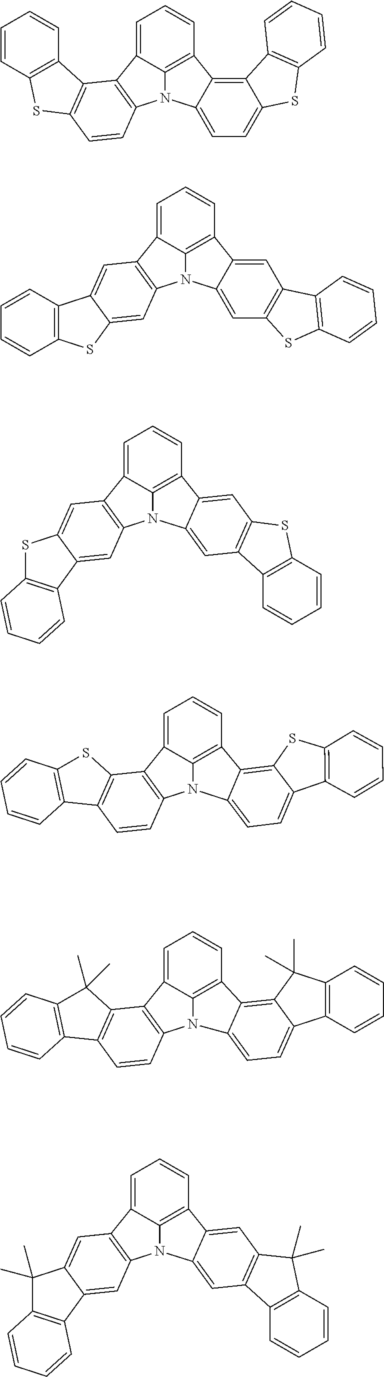 [SMILES: c1ccc2c(c1)sc1ccc3c(c4cccc5c6c7c(ccc6n3c45)sc3ccccc37)c12 ; c1ccc2c(c1)sc1cc3c(cc12)c1cccc2c4cc5c(cc4n3c12)sc1ccccc15 ; c1ccc2c(c1)sc1cc3c4cccc5c6cc7sc8ccccc8c7cc6n(c3cc12)c45 ; c1ccc2c(c1)sc1c2ccc2c1c1cccc3c4c5sc6ccccc6c5ccc4n2c13 ; CC1(C)c2ccccc2-c2ccc3c(c21)c1cccc2c4c5c(ccc4n3c12)-c1ccccc1C5(C)C ; CC1(C)c2ccccc2-c2cc3c(cc21)c1cccc2c4cc5c(cc4n3c12)-c1ccccc1C5(C)C]

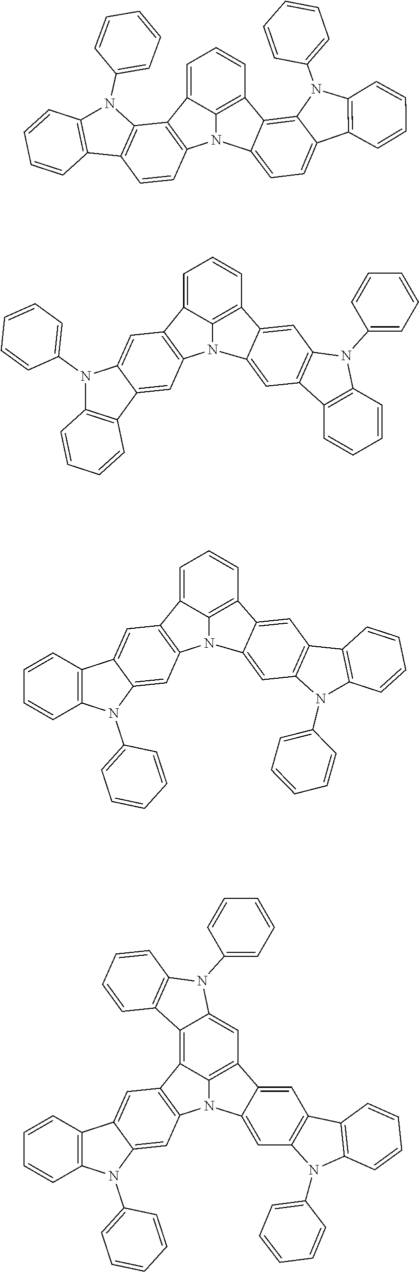 [SMILES: c1ccc(-n2c3ccccc3c3ccc4c(c5cccc6c7c8c(ccc7n4c56)c4ccccc4n8-c4ccccc4)c32)cc1 ; c1ccc(-n2c3ccccc3c3cc4c(cc32)c2cccc3c5cc6c(cc5n4c23)c2ccccc2n6-c2ccccc2)cc1 ; c1ccc(-n2c3ccccc3c3cc4c5cccc6c7cc8c9ccccc9n(-c9ccccc9)c8cc7n(c4cc32)c56)cc1 ; c1ccc(-n2c3ccccc3c3cc4c5cc6c(c7ccccc7n6-c6ccccc6)c6c7cc8c9ccccc9n(-c9ccccc9)c8cc7n(c4cc32)c56)cc1]

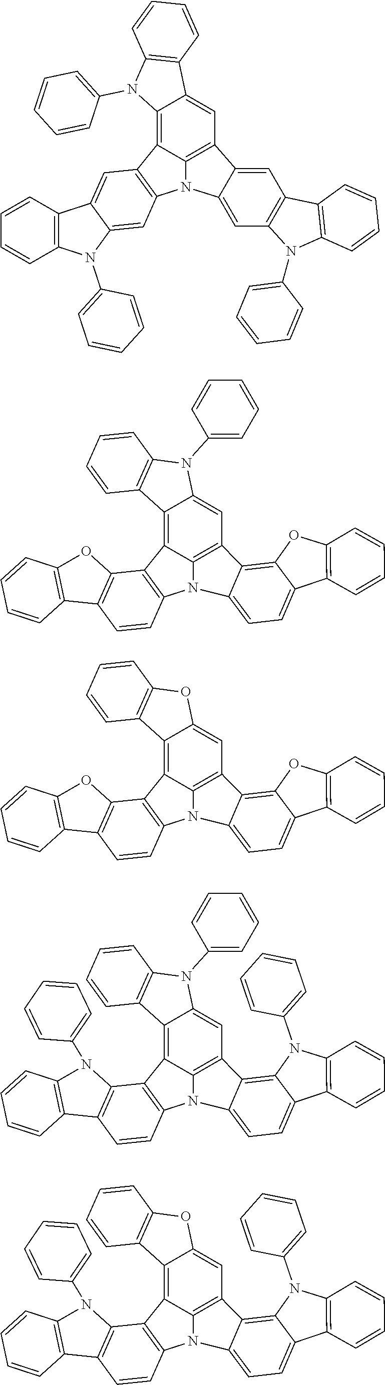

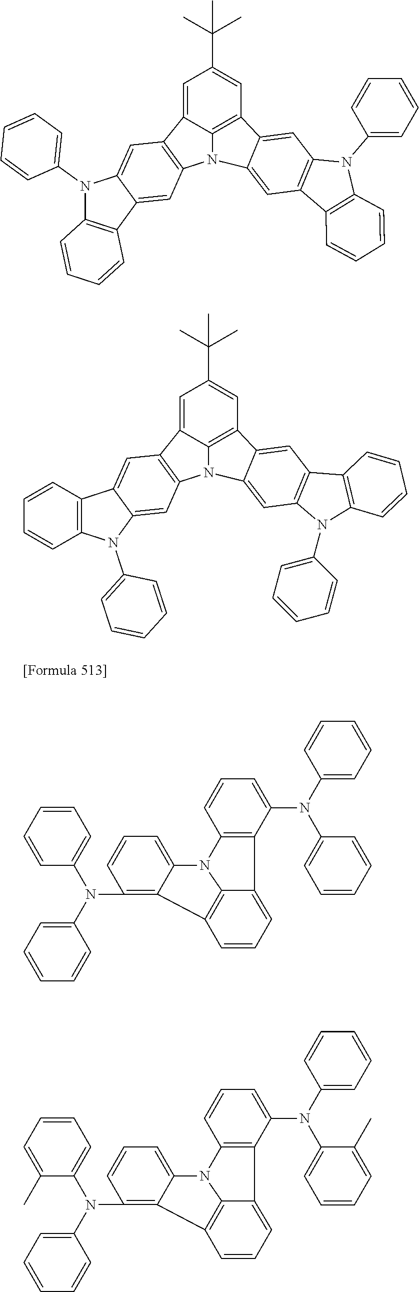 [SMILES: CC(C)(C)c1cc2c3cc4c(cc3n3c5cc6c7ccccc7n(-c7ccccc7)c6cc5c(c1)c23)c1ccccc1n4-c1ccccc1 ; CC(C)(C)c1cc2c3cc4c5ccccc5n(-c5ccccc5)c4cc3n3c4cc5c(cc4c(c1)c23)c1ccccc1n5-c1ccccc1 ; c1ccc(N(c2ccccc2)c2cccc3c2c2cccc4c5c(N(c6ccccc6)c6ccccc6)cccc5n3c24)cc1 ; Cc1ccccc1N(c1ccccc1)c1cccc2c1c1cccc3c4c(N(c5ccccc5)c5ccccc5C)cccc4n2c13]

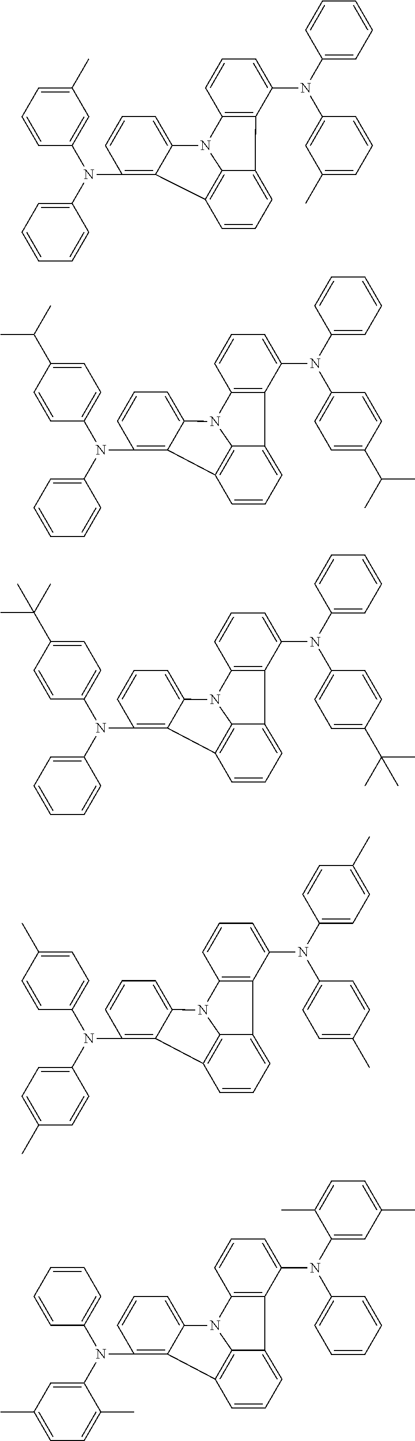

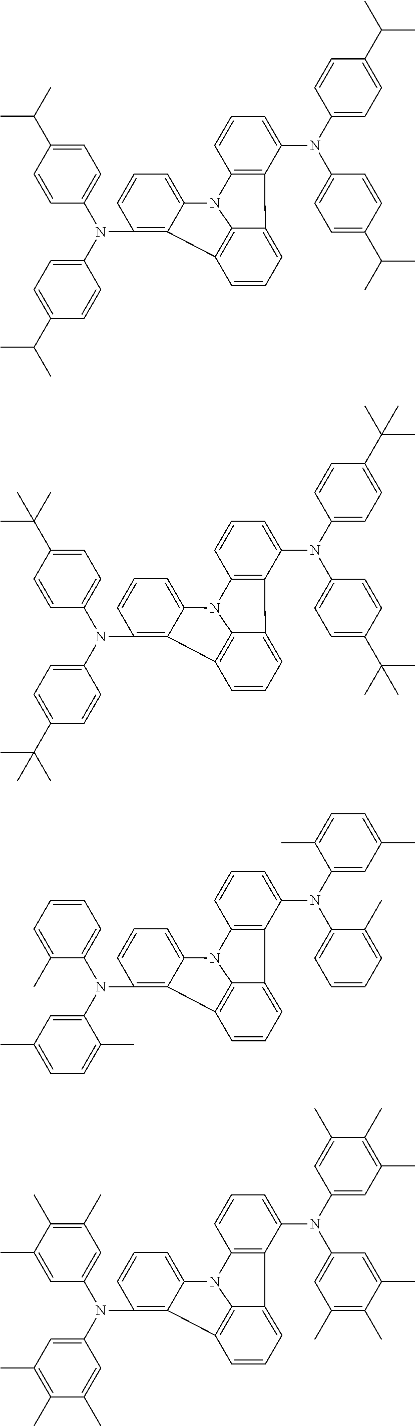

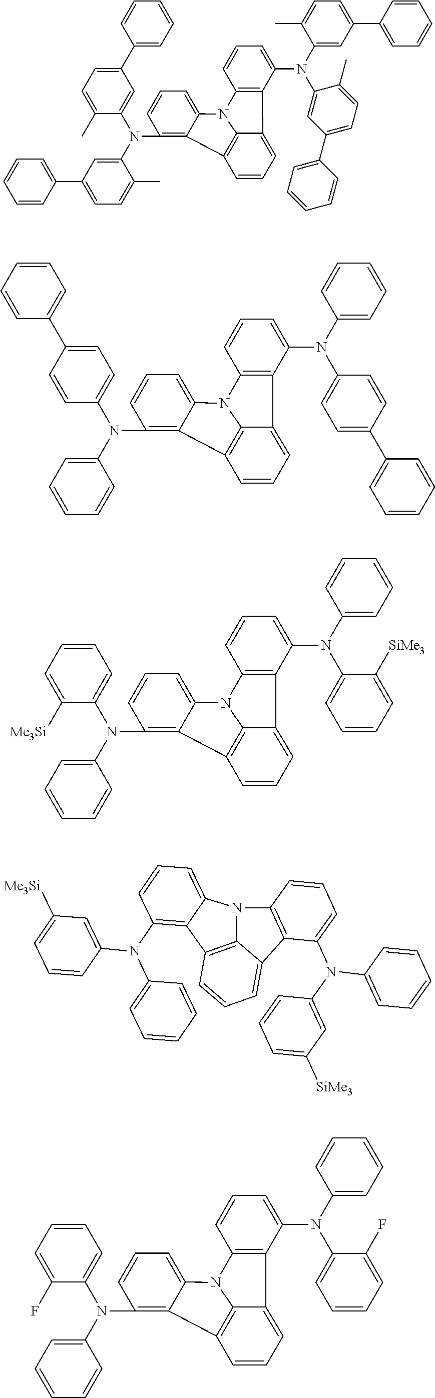

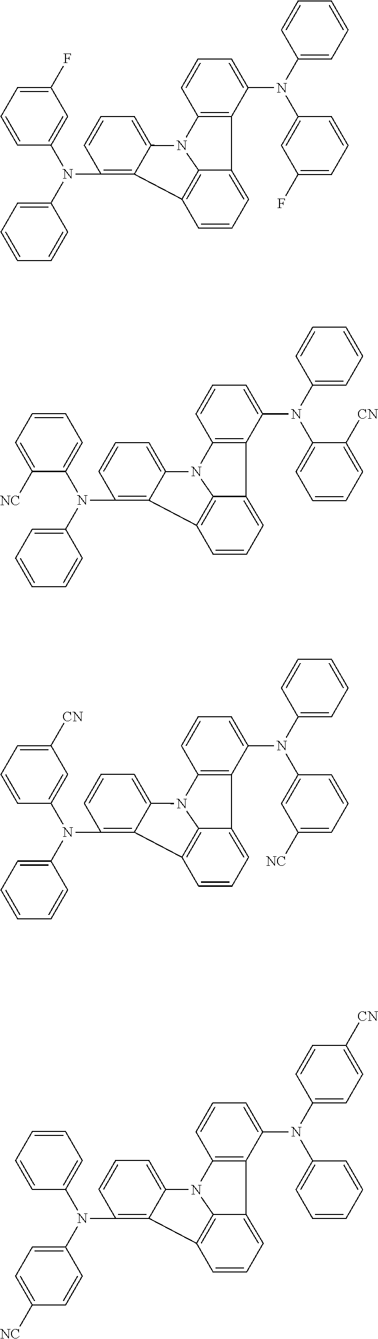 [SMILES: Fc1cccc(N(c2ccccc2)c2cccc3c2c2cccc4c5c(N(c6ccccc6)c6cccc(F)c6)cccc5n3c24)c1 ; N#Cc1ccccc1N(c1ccccc1)c1cccc2c1c1cccc3c4c(N(c5ccccc5)c5ccccc5C#N)cccc4n2c13 ; N#Cc1cccc(N(c2ccccc2)c2cccc3c2c2cccc4c5c(N(c6ccccc6)c6cccc(C#N)c6)cccc5n3c24)c1 ; N#Cc1ccc(N(c2ccccc2)c2cccc3c2c2cccc4c5c(N(c6ccccc6)c6ccc(C#N)cc6)cccc5n3c24)cc1]

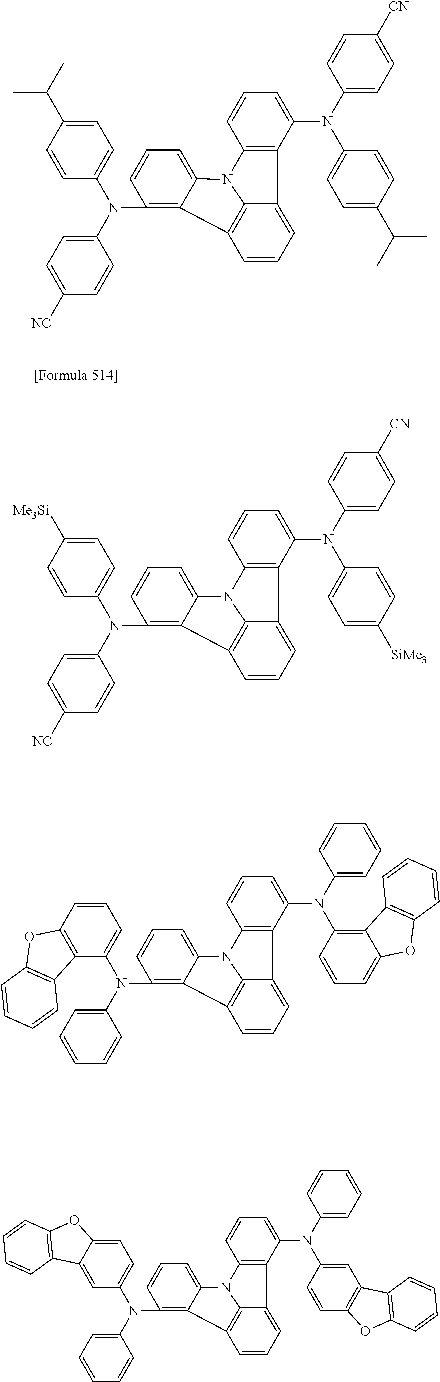 [SMILES: CC(C)c1ccc(N(c2ccc(C#N)cc2)c2cccc3c2c2cccc4c5c(N(c6ccc(C#N)cc6)c6ccc(C(C)C)cc6)cccc5n3c24)cc1 ; C[Si](C)(C)c1ccc(N(c2ccc(C#N)cc2)c2cccc3c2c2cccc4c5c(N(c6ccc(C#N)cc6)c6ccc([Si](C)(C)C)cc6)cccc5n3c24)cc1 ; c1ccc(N(c2cccc3oc4ccccc4c23)c2cccc3c2c2cccc4c5c(N(c6ccccc6)c6cccc7oc8ccccc8c67)cccc5n3c24)cc1 ; c1ccc(N(c2ccc3oc4ccccc4c3c2)c2cccc3c2c2cccc4c5c(N(c6ccccc6)c6ccc7oc8ccccc8c7c6)cccc5n3c24)cc1]

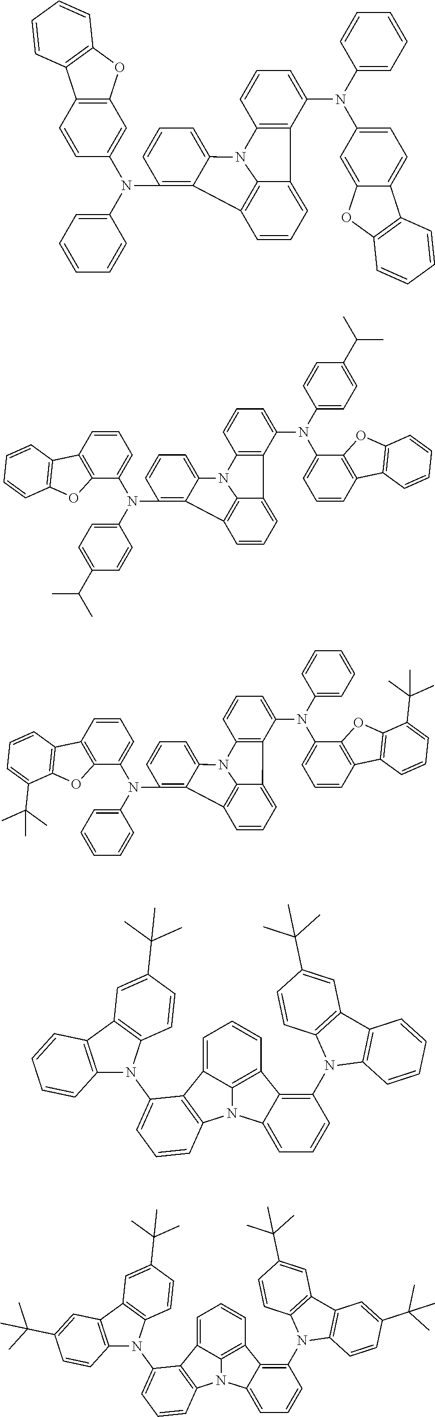

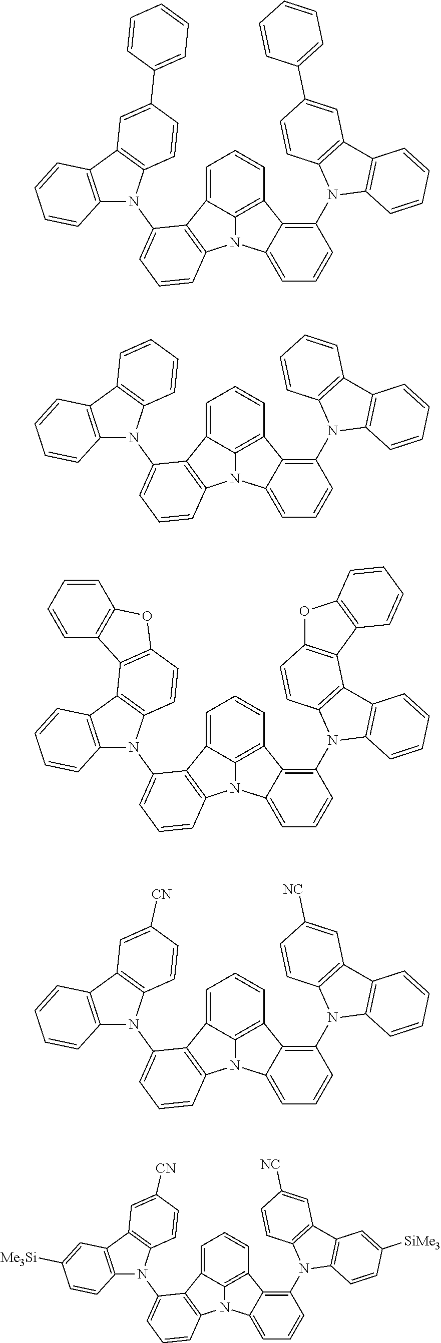 [SMILES: c1ccc(-c2ccc3c(c2)c2ccccc2n3-c2cccc3c2c2cccc4c5c(-n6c7ccccc7c7cc(-c8ccccc8)ccc76)cccc5n3c24)cc1 ; c1ccc2c(c1)c1ccccc1n2-c1cccc2c1c1cccc3c4c(-n5c6ccccc6c6ccccc65)cccc4n2c13 ; c1ccc2c(c1)oc1ccc3c(c4ccccc4n3-c3cccc4c3c3cccc5c6c(-n7c8ccccc8c8c9c(ccc87)oc7ccccc79)cccc6n4c35)c12 ; N#Cc1ccc2c(c1)c1ccccc1n2-c1cccc2c1c1cccc3c4c(-n5c6ccccc6c6cc(C#N)ccc65)cccc4n2c13 ; C[Si](C)(C)c1ccc2c(c1)c1cc(C#N)ccc1n2-c1cccc2c1c1cccc3c4c(-n5c6ccc(C#N)cc6c6cc([Si](C)(C)C)ccc65)cccc4n2c13]

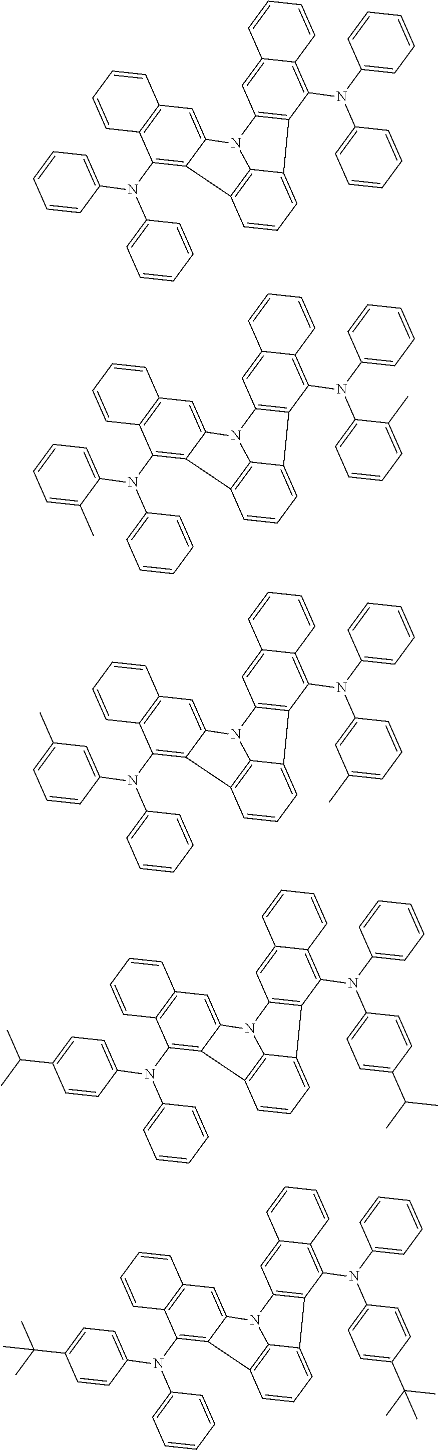

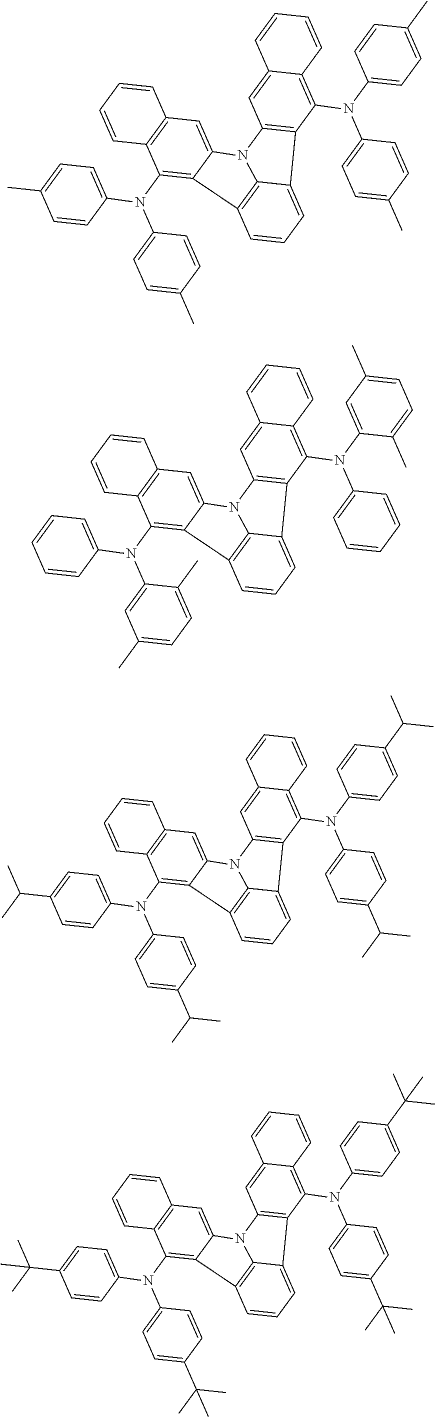

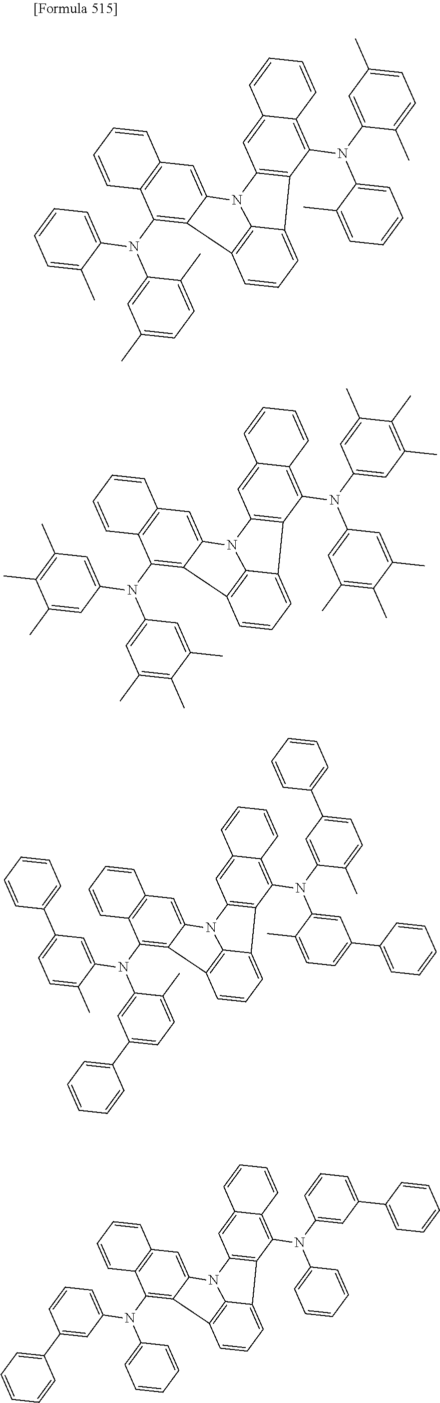

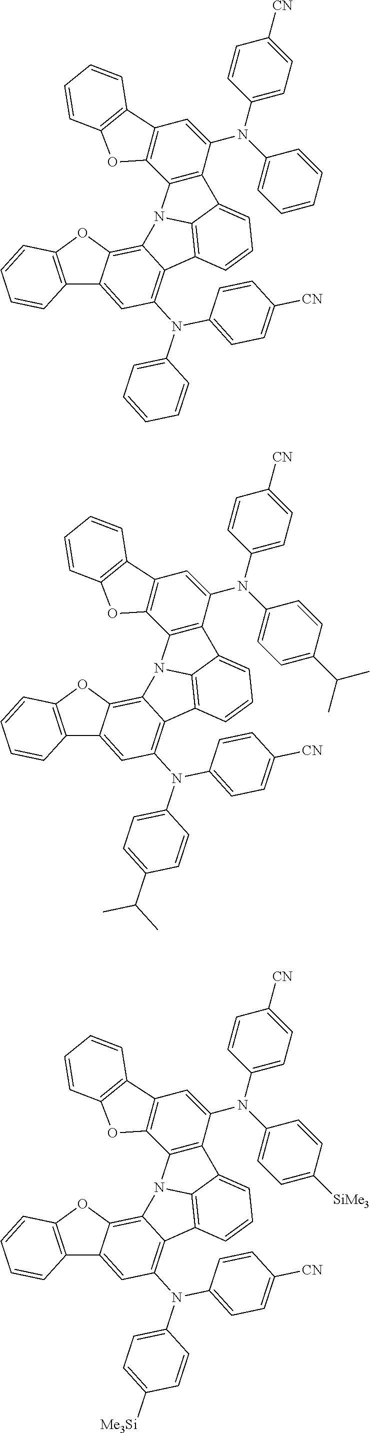 [SMILES: N#Cc1ccc(N(c2ccccc2)c2cc3c4ccccc4oc3c3c2c2cccc4c5c(N(c6ccccc6)c6ccc(C#N)cc6)cc6c7ccccc7oc6c5n3c24)cc1 ; CC(C)c1ccc(N(c2ccc(C#N)cc2)c2cc3c4ccccc4oc3c3c2c2cccc4c5c(N(c6ccc(C#N)cc6)c6ccc(C(C)C)cc6)cc6c7ccccc7oc6c5n3c24)cc1 ; C[Si](C)(C)c1ccc(N(c2ccc(C#N)cc2)c2cc3c4ccccc4oc3c3c2c2cccc4c5c(N(c6ccc(C#N)cc6)c6ccc([Si](C)(C)C)cc6)cc6c7ccccc7oc6c5n3c24)cc1]

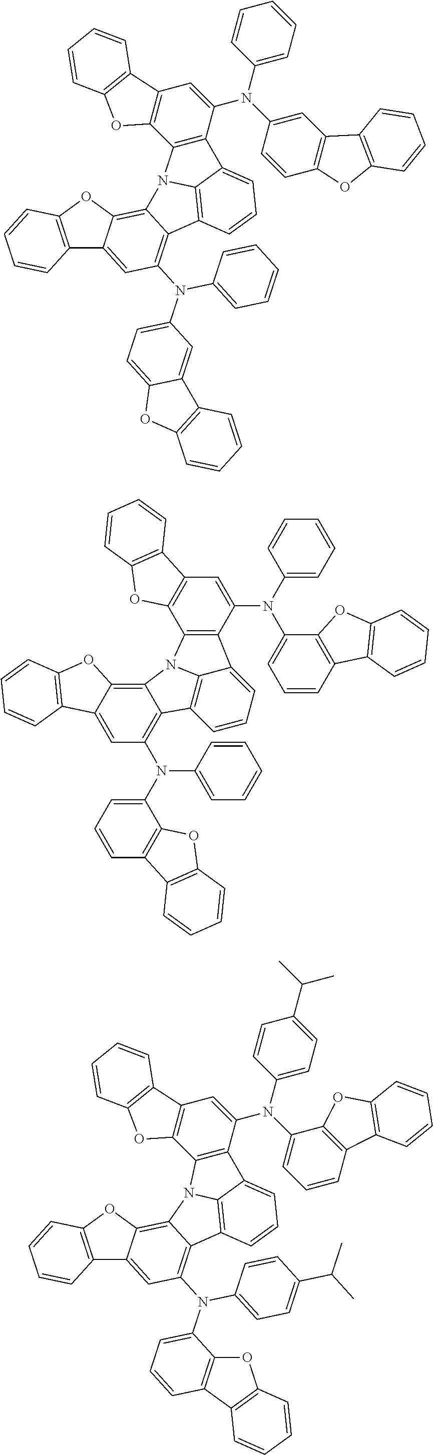 [SMILES: c1ccc(N(c2ccc3oc4ccccc4c3c2)c2cc3c4ccccc4oc3c3c2c2cccc4c5c(N(c6ccccc6)c6ccc7oc8ccccc8c7c6)cc6c7ccccc7oc6c5n3c24)cc1 ; c1ccc(N(c2cccc3c2oc2ccccc23)c2cc3c4ccccc4oc3c3c2c2cccc4c5c(N(c6ccccc6)c6cccc7c6oc6ccccc67)cc6c7ccccc7oc6c5n3c24)cc1 ; CC(C)c1ccc(N(c2cccc3c2oc2ccccc23)c2cc3c4ccccc4oc3c3c2c2cccc4c5c(N(c6ccc(C(C)C)cc6)c6cccc7c6oc6ccccc67)cc6c7ccccc7oc6c5n3c24)cc1]

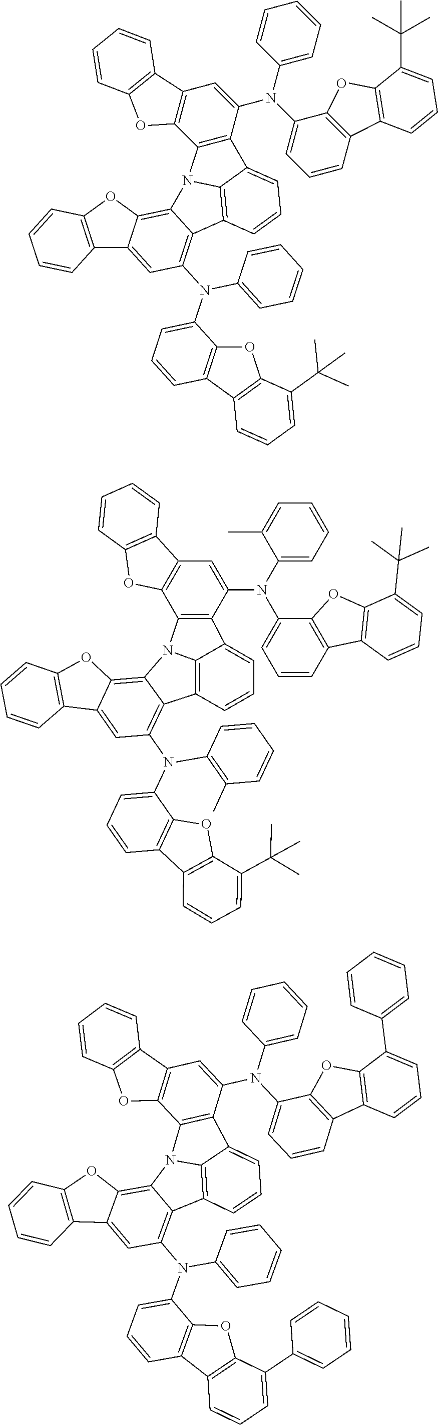 [SMILES: CC(C)(C)c1cccc2c1oc1c(N(c3ccccc3)c3cc4c5ccccc5oc4c4c3c3cccc5c6c(N(c7ccccc7)c7cccc8c7oc7c(C(C)(C)C)cccc78)cc7c8ccccc8oc7c6n4c35)cccc12 ; Cc1ccccc1N(c1cccc2c1oc1c(C(C)(C)C)cccc12)c1cc2c3ccccc3oc2c2c1c1cccc3c4c(N(c5ccccc5C)c5cccc6c5oc5c(C(C)(C)C)cccc56)cc5c6ccccc6oc5c4n2c13 ; c1ccc(-c2cccc3c2oc2c(N(c4ccccc4)c4cc5c6ccccc6oc5c5c4c4cccc6c7c(N(c8ccccc8)c8cccc9c8oc8c(-c%10ccccc%10)cccc89)cc8c9ccccc9oc8c7n5c46)cccc23)cc1]

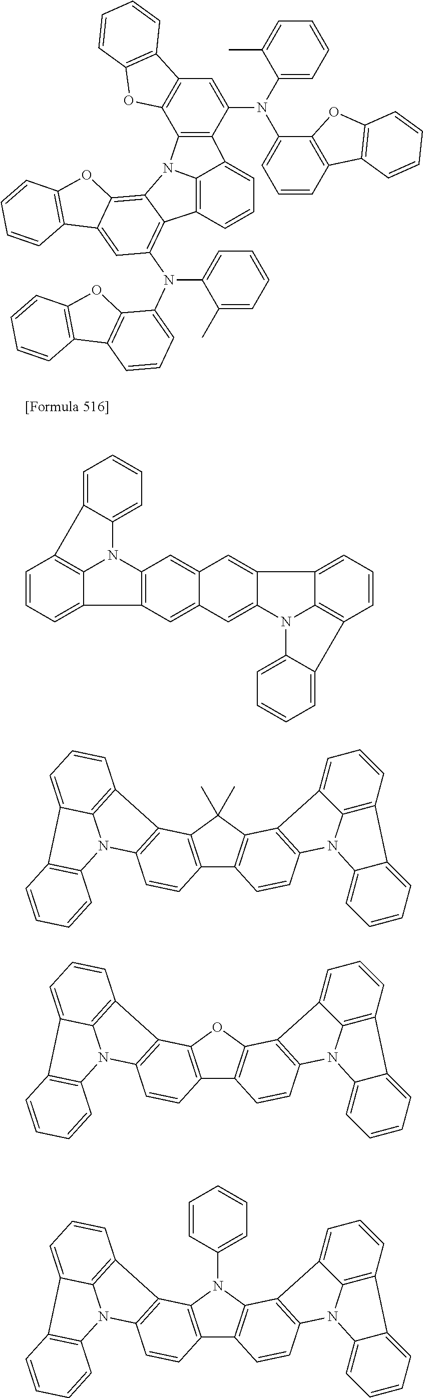

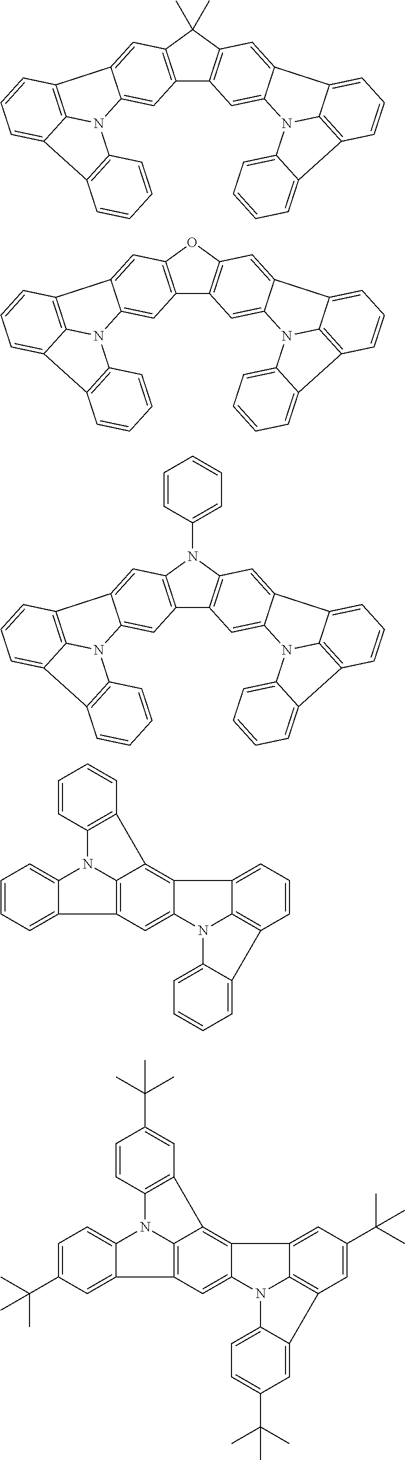

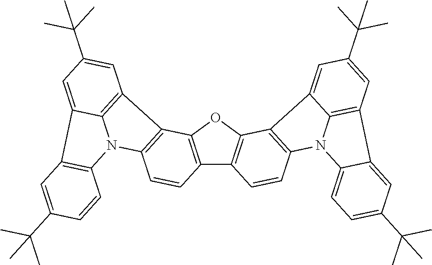 [SMILES: CC(C)(C)c1ccc2c(c1)c1cc(C(C)(C)C)cc3c4c5oc6c(ccc7c6c6cc(C(C)(C)C)cc8c9cc(C(C)(C)C)ccc9n7c86)c5ccc4n2c13]

### Compound Represented by Formula (5)

The compound represented by the formula (5) will be described. The compound represented by the formula (5) corresponds to a compound represented by the formula (41-3).

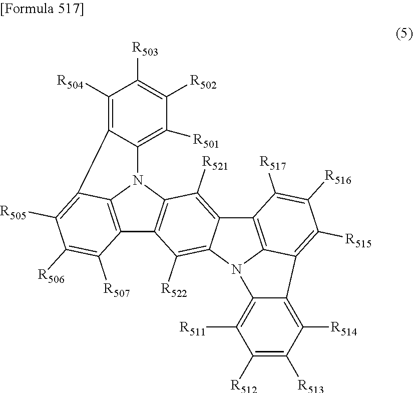

In the formula (5):
at least one combination of adjacent two or more of R₅₀₁ to R₅₀₇ and R₅₁₁ to R₅₁₇ are mutually bonded to form a substituted or unsubstituted monocyclic ring, mutually bonded to form a substituted or unsubstituted fused ring, or not mutually bonded; and
R₅₀₁ to R₅₀₇ and R₅₁₁ to R₅₁₇ forming neither the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.
R₅₂₁ and R₅₂₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

“A combination of adjacent two or more of R₅₀₁ to R₅₀₇ and R₅₁₁ to R₅₁₇” refers to, for instance, a combination of R₅₀₁ and R₅₀₂, a combination of R₅₀₂ and R₅₀₃, a combination of R₅₀₃ and R₅₀₄, a combination of R₅₀₅ and R₅₀₆, a combination of R₅₀₆ and R₅₀₇, and a combination of R₅₀₁, R₅₀₂, and R₅₀₃.

In an exemplary embodiment, at least one, preferably two of R₅₀₁ to R₅₀₇ and R₅₁₁ to R₅₁₇ are groups represented by —N(R₉₀₆)(R₉₀₇).

In an exemplary embodiment, R₅₀₁ to R₅₀₇ and R₅₁₁ to R₅₁₇ are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, the compound represented by the formula (5) is a compound represented by a formula (52) below.

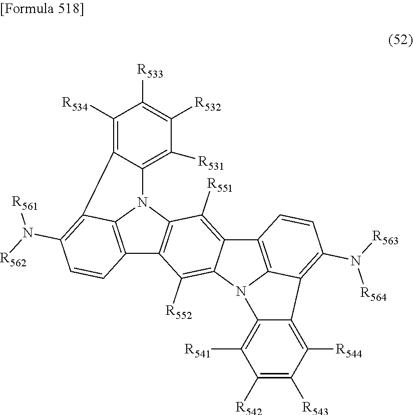

In the formula (52):
at least one combination of adjacent two or more of R₅₃₁ to R₅₃₄ and R₅₄₁ to R₅₄₄ are mutually bonded to form a substituted or unsubstituted monocyclic ring, mutually bonded to form a substituted or unsubstituted fused ring, or not mutually bonded;
R₅₃₁ to R₅₃₄, R₅₄₁ to R₅₄₄ forming neither the monocyclic ring nor the fused ring, and R₅₅₁ and R₅₅₂ are each independently a hydrogen atom, a substituted or unsubstituted aryl group having 6 to 50 ring carbon atoms, or a substituted or unsubstituted heterocyclic group having 5 to 50 ring atoms; and
R₅₆₁ to R₅₆₄ are each independently a substituted or unsubstituted aryl group having 6 to 50 ring carbon atoms, or a substituted or unsubstituted heterocyclic group having 5 to 50 ring atoms.

In an exemplary embodiment, the compound represented by the formula (5) is a compound represented by a formula (53) below.

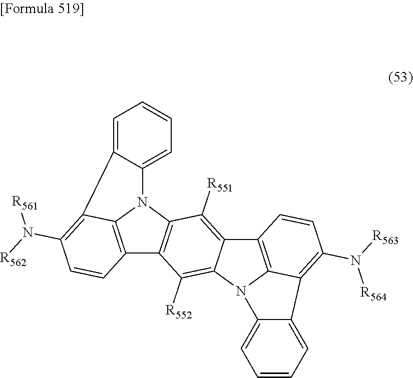

In the formula (53), R₅₅₁, R₅₅₂ and R₅₆₁ to R₅₆₄ each independently represent the same as R₅₅₁, R₅₅₂ and R₅₆₁ to R₅₆₄ in the formula (52).

In an exemplary embodiment, R₅₆₁ to R₅₆₄ in the formulae (52) and (53) are each independently a substituted or unsubstituted aryl group having 6 to 50 ring carbon atoms (preferably a phenyl group).

In an exemplary embodiment, R₅₂₁ and R₅₂₂ in the formula (5) and R₅₅₁ and R₅₅₂ in the formulae (52) and (53) are each a hydrogen atom.

In an exemplary embodiment, the substituent for the “substituted or unsubstituted” group in the formulae (5), (52) and (53) is a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

Specific examples of the compound represented by the formula (5) include compounds shown below.

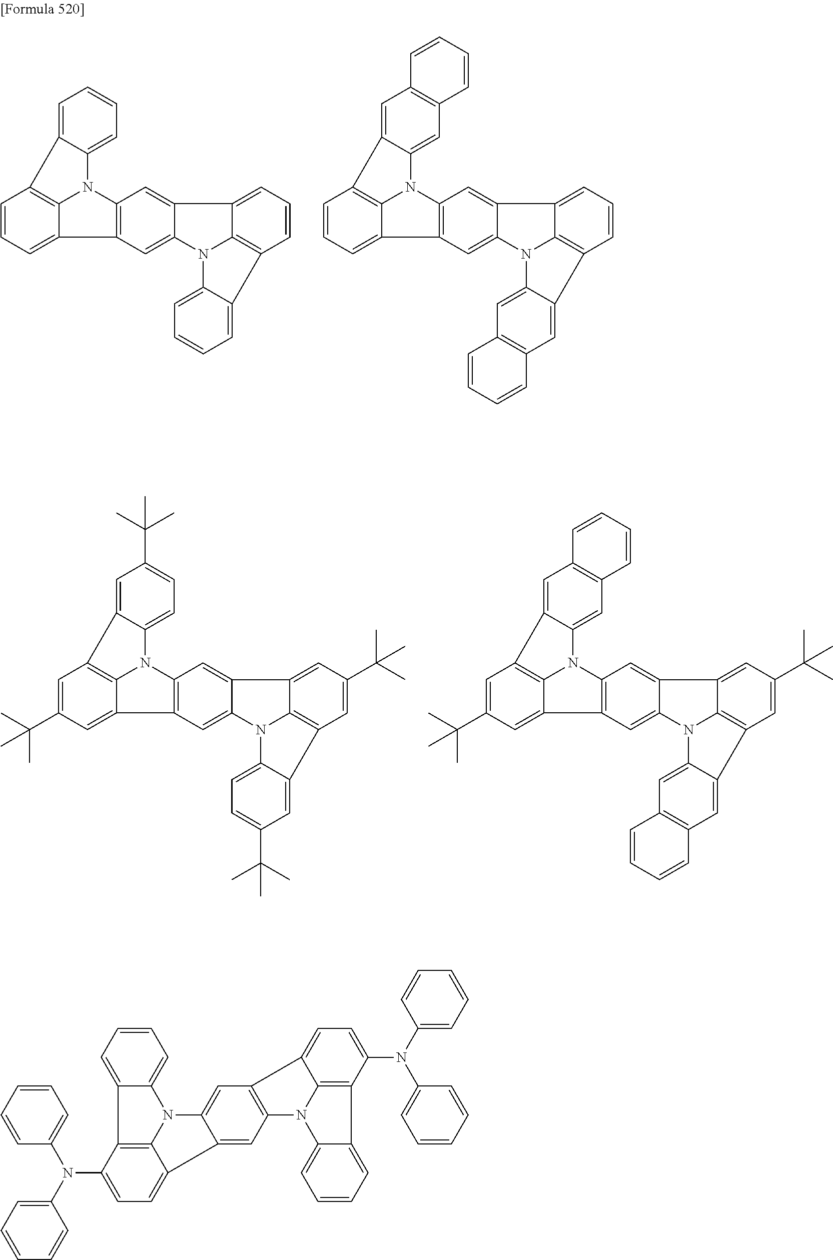

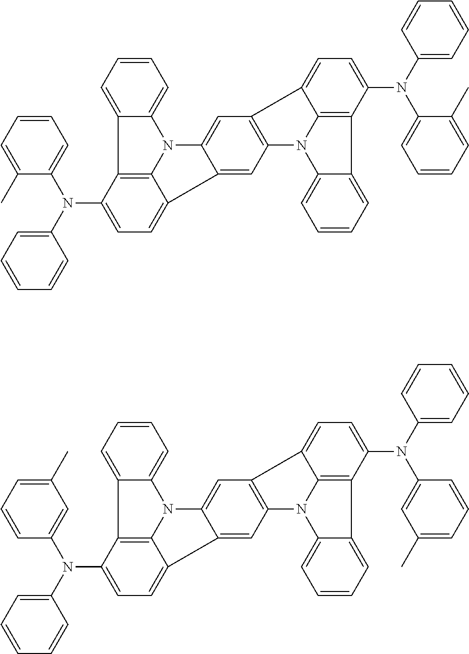

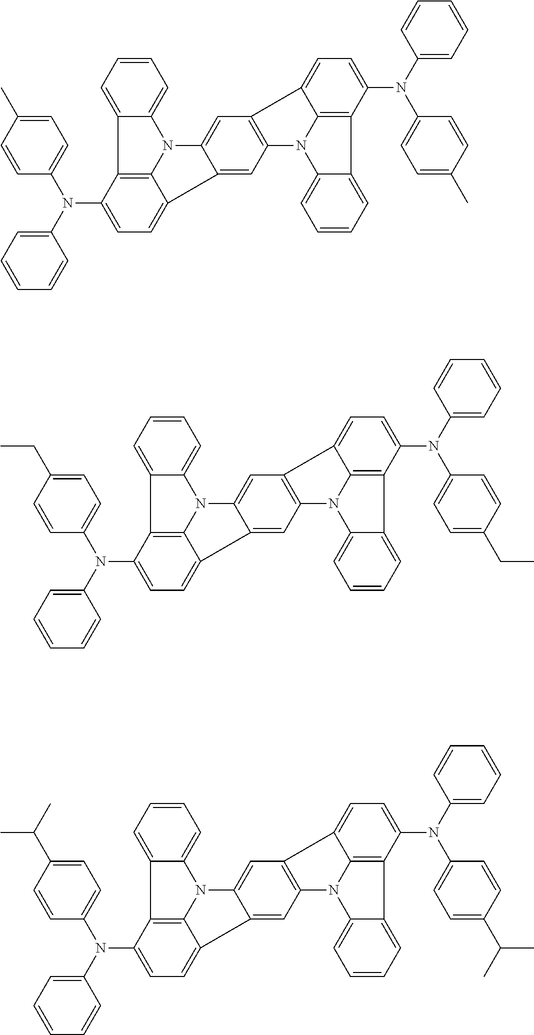

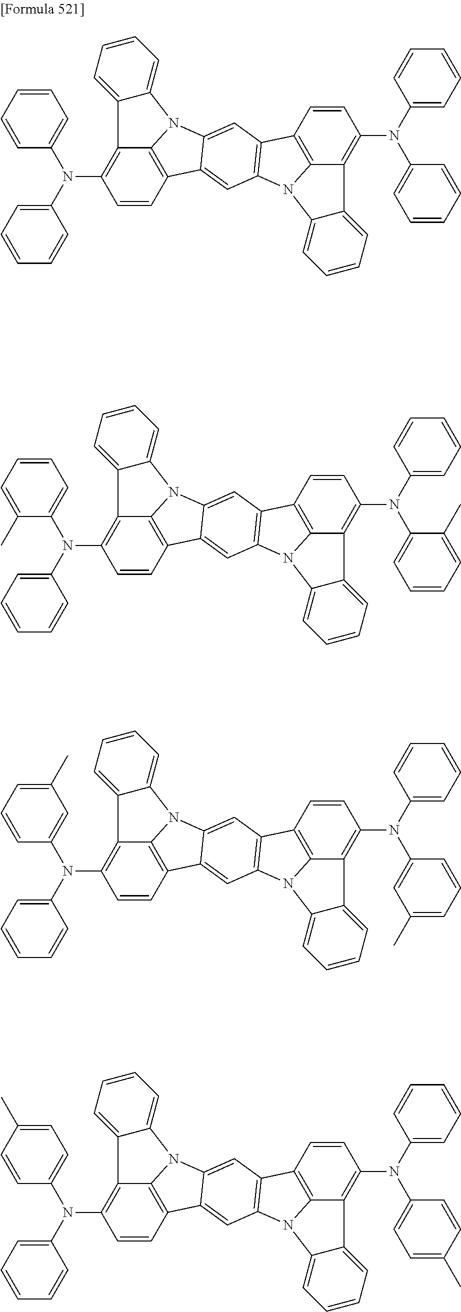

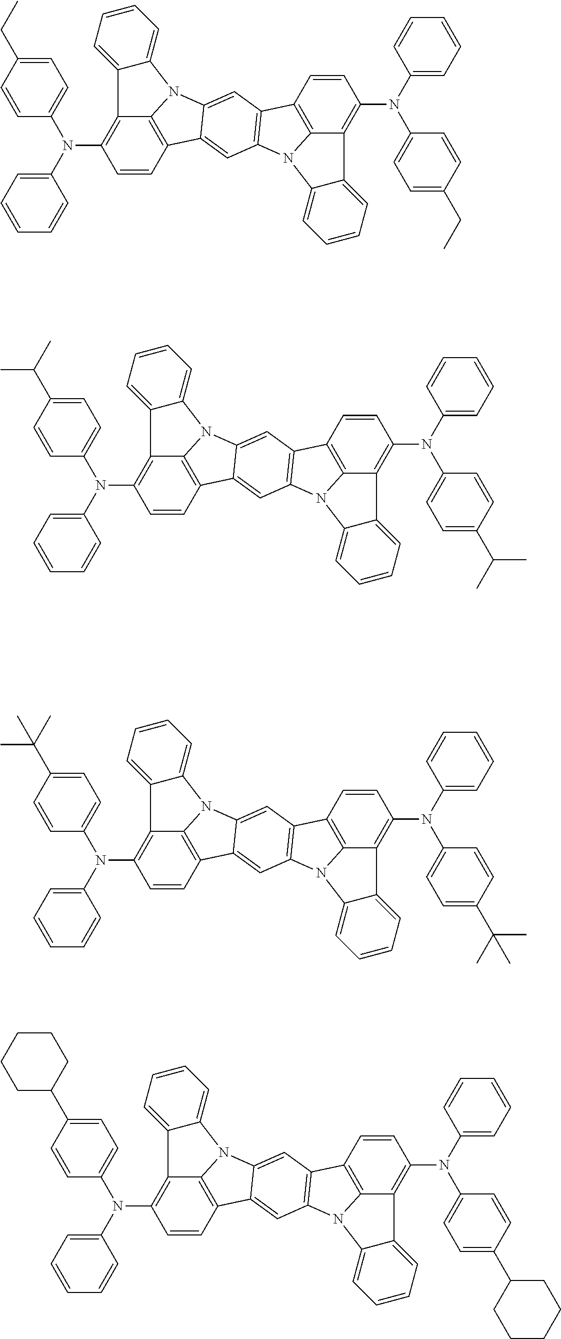

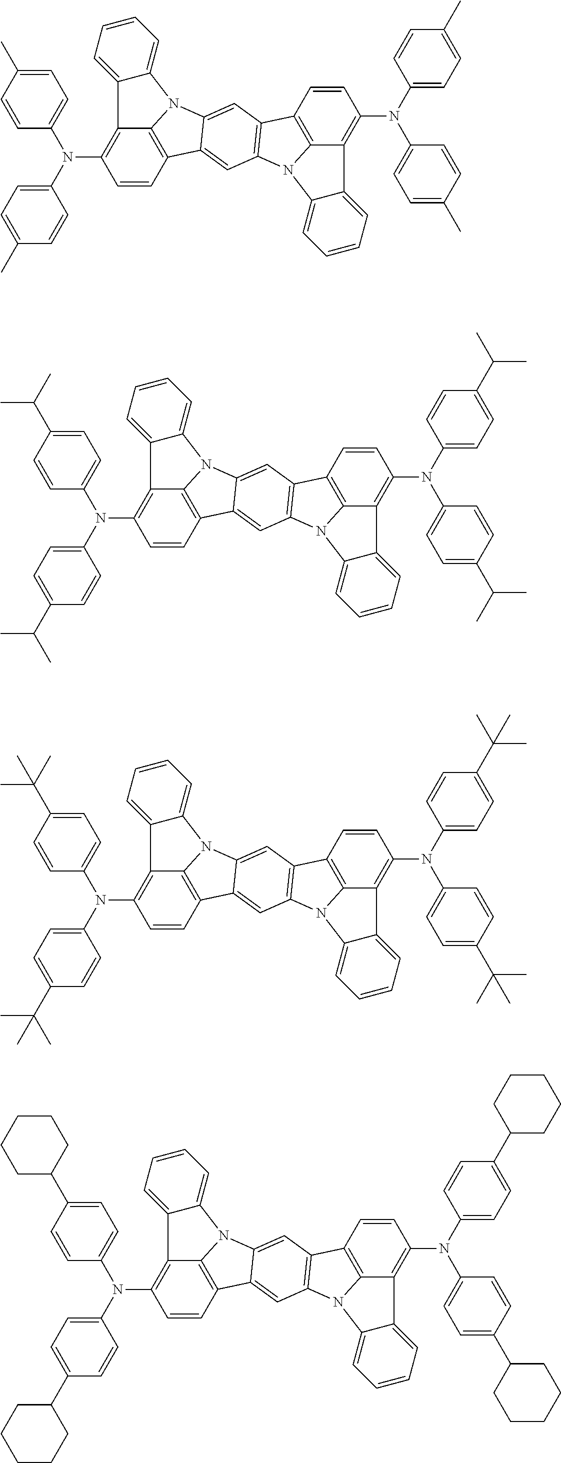

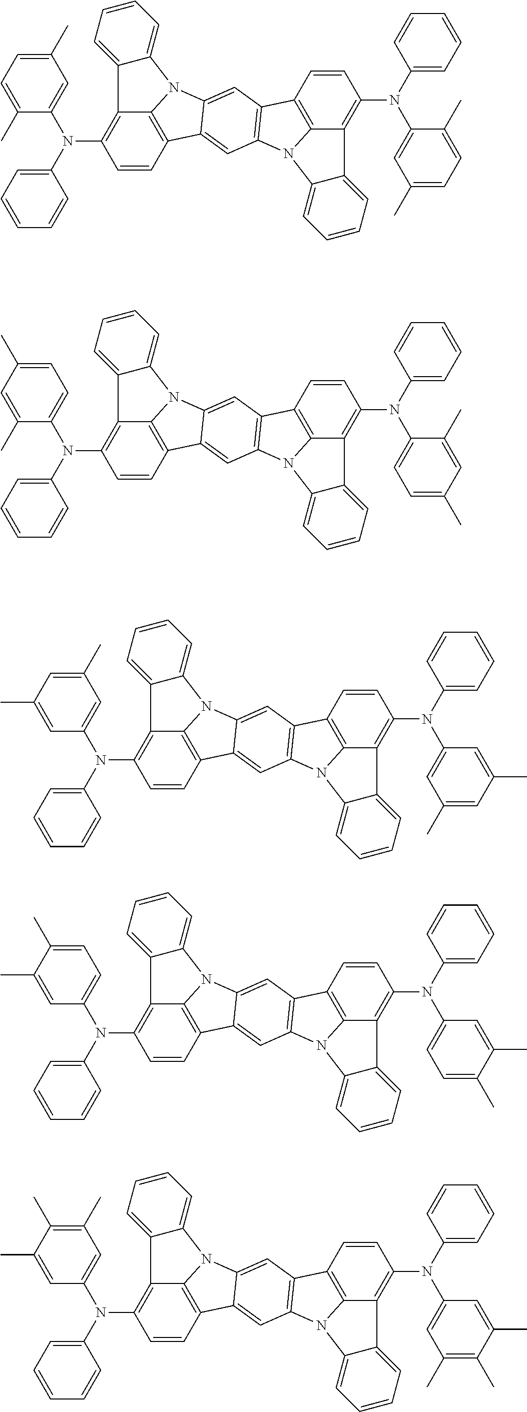

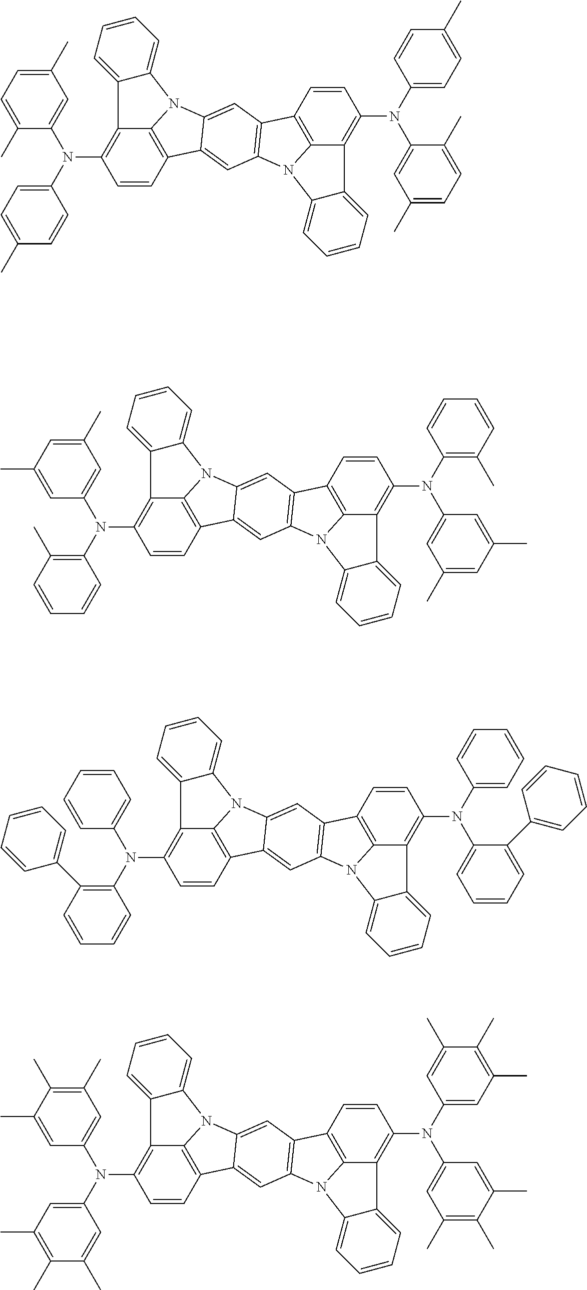

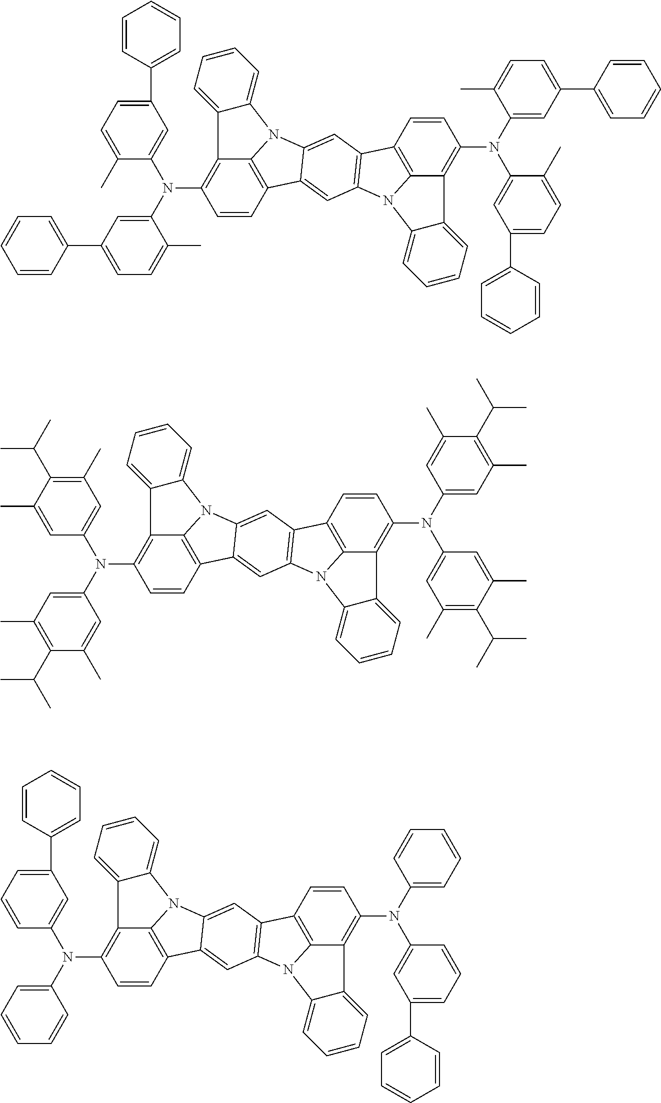

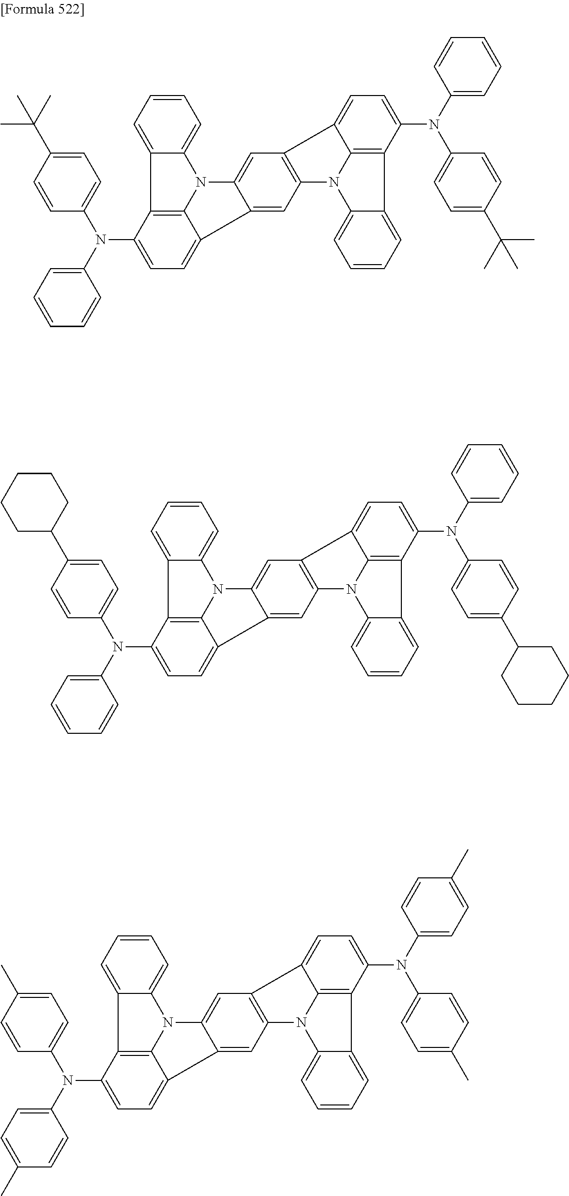

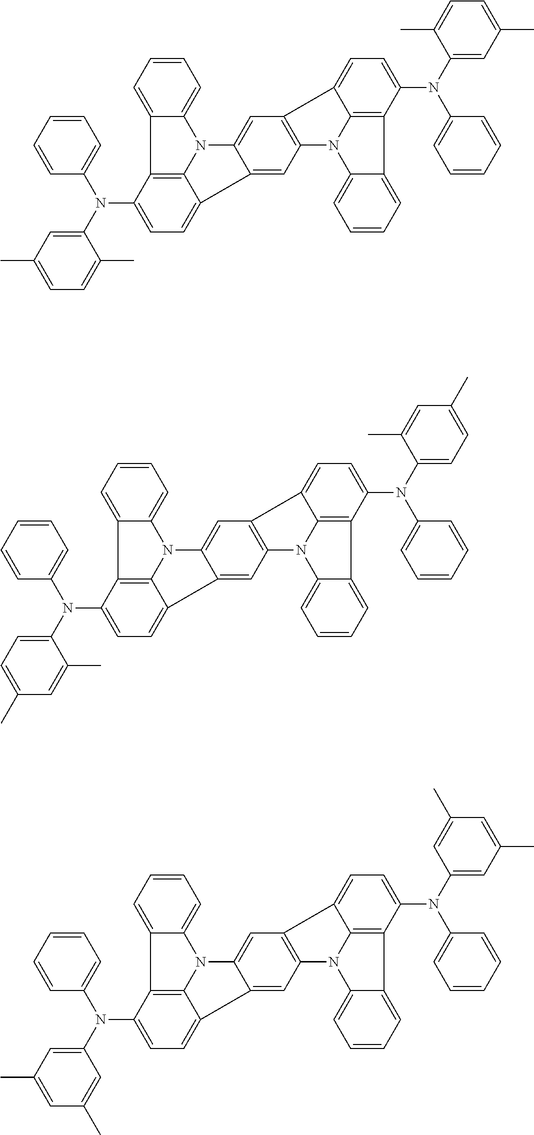

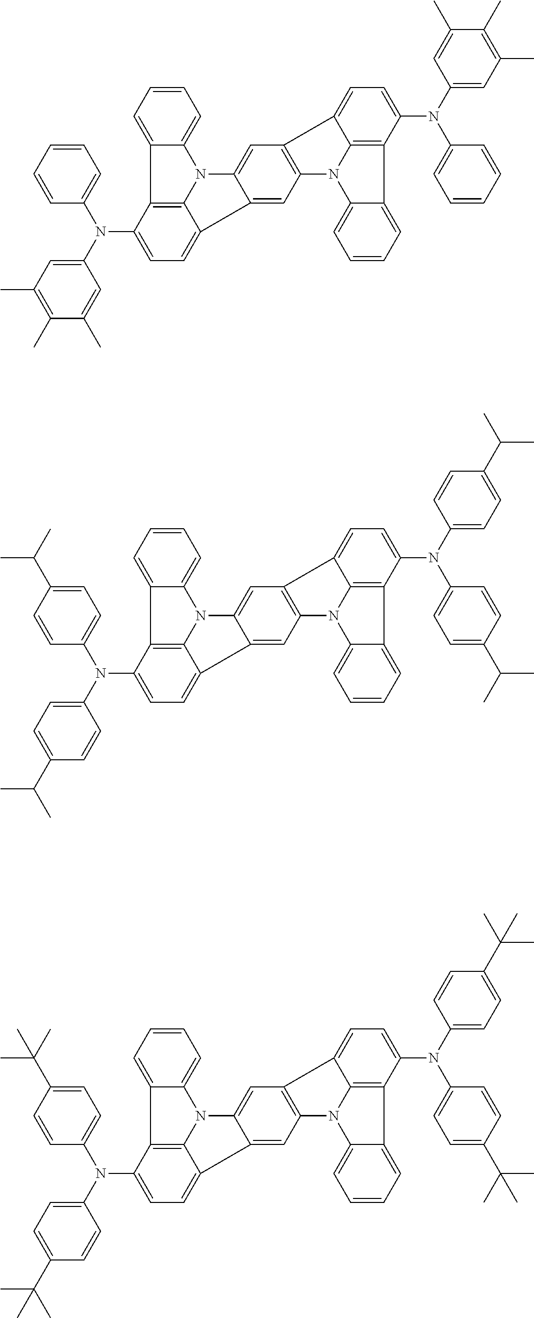

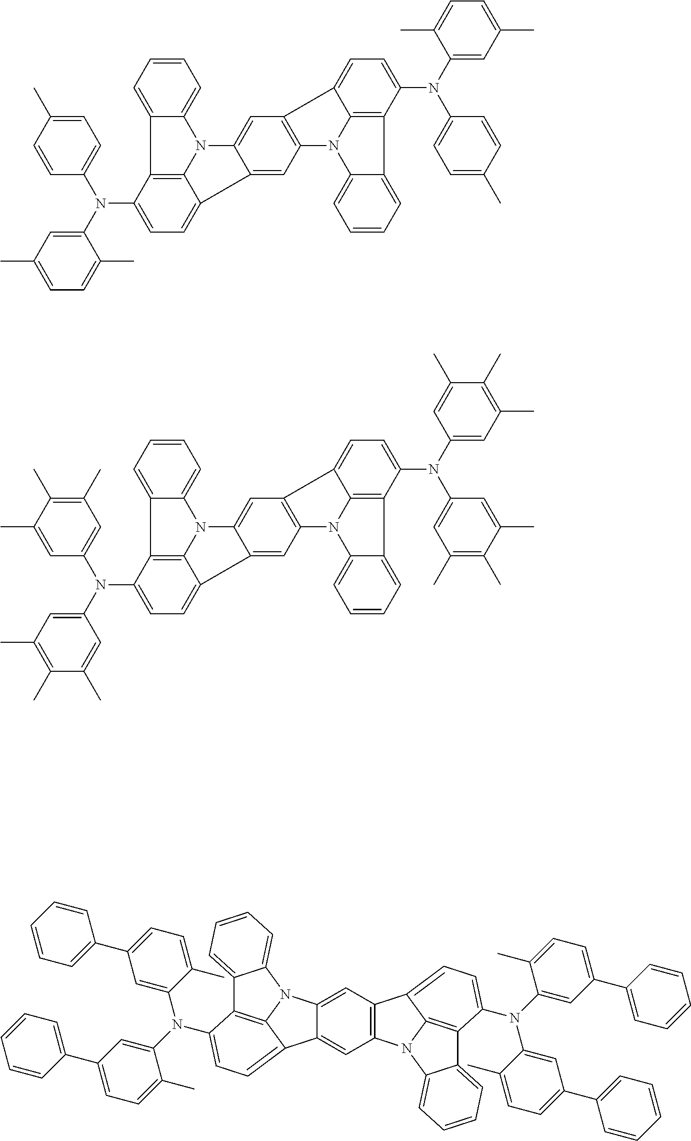

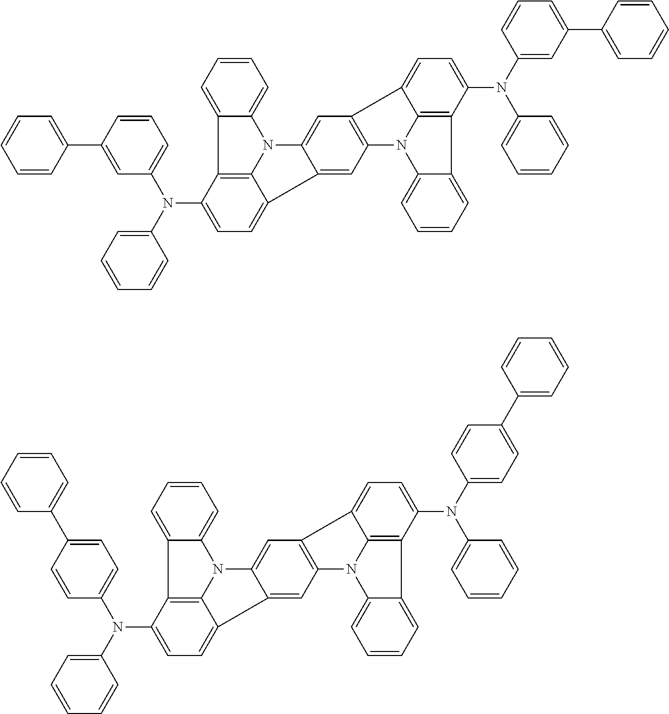

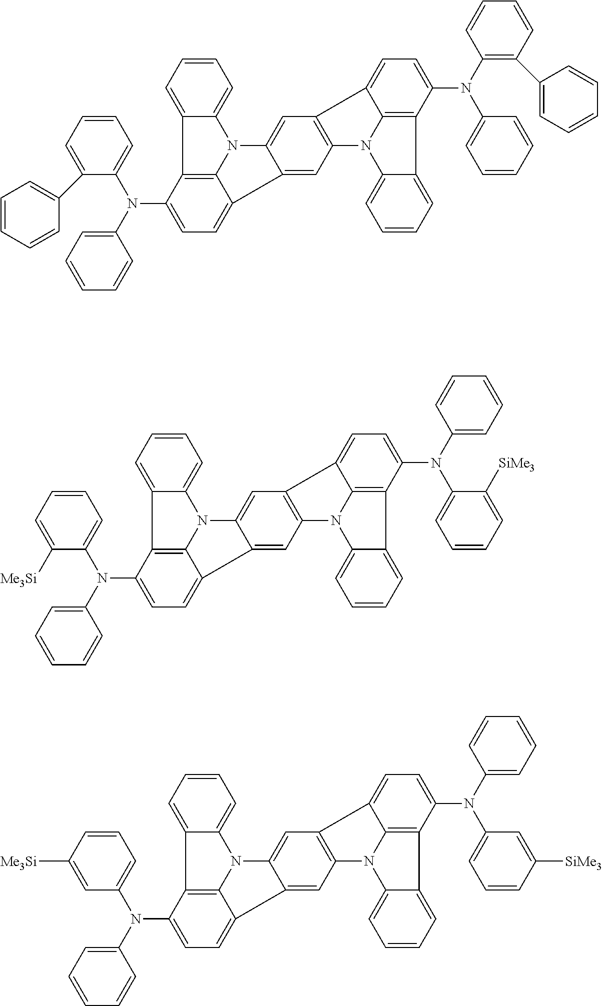

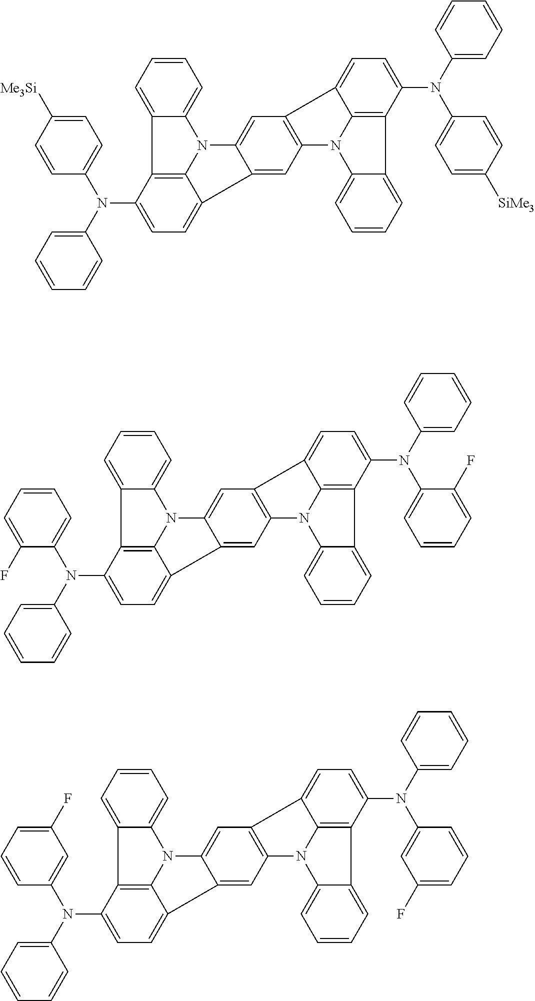

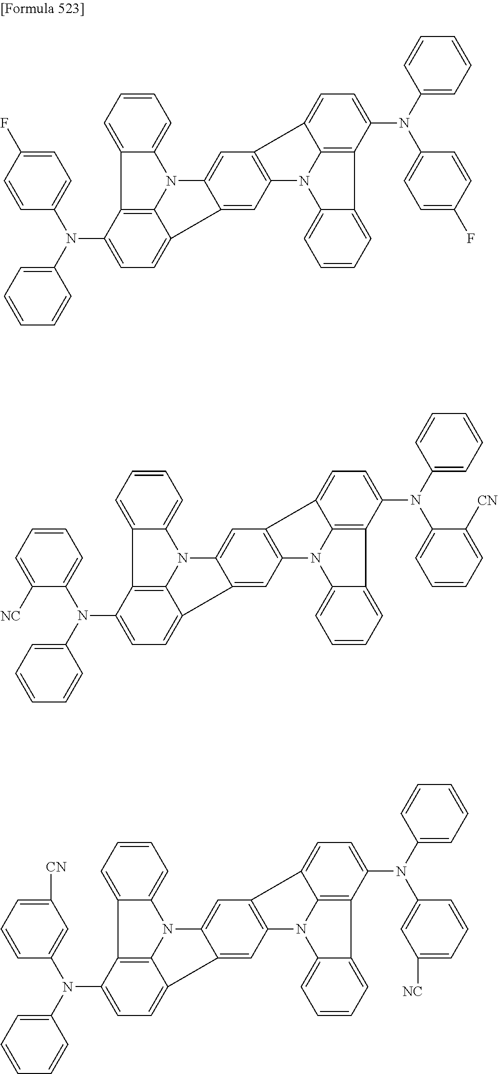

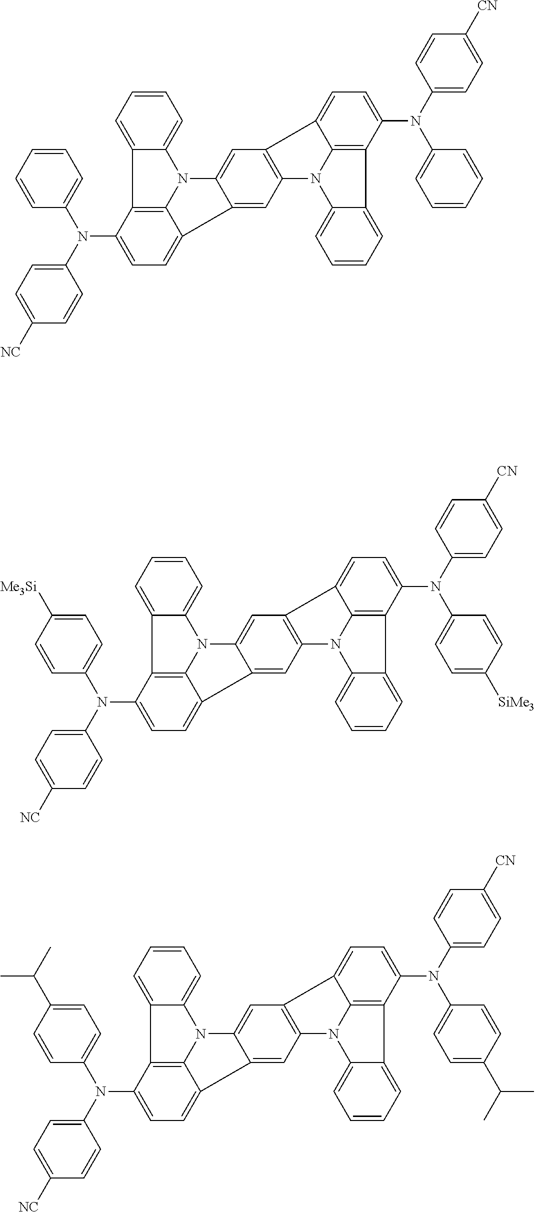

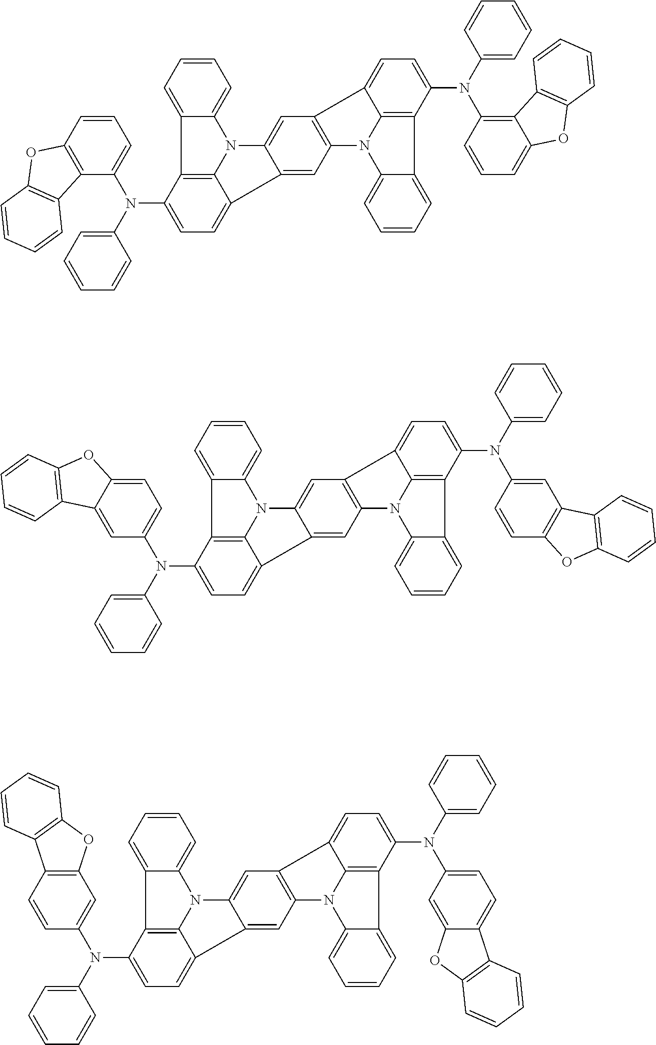

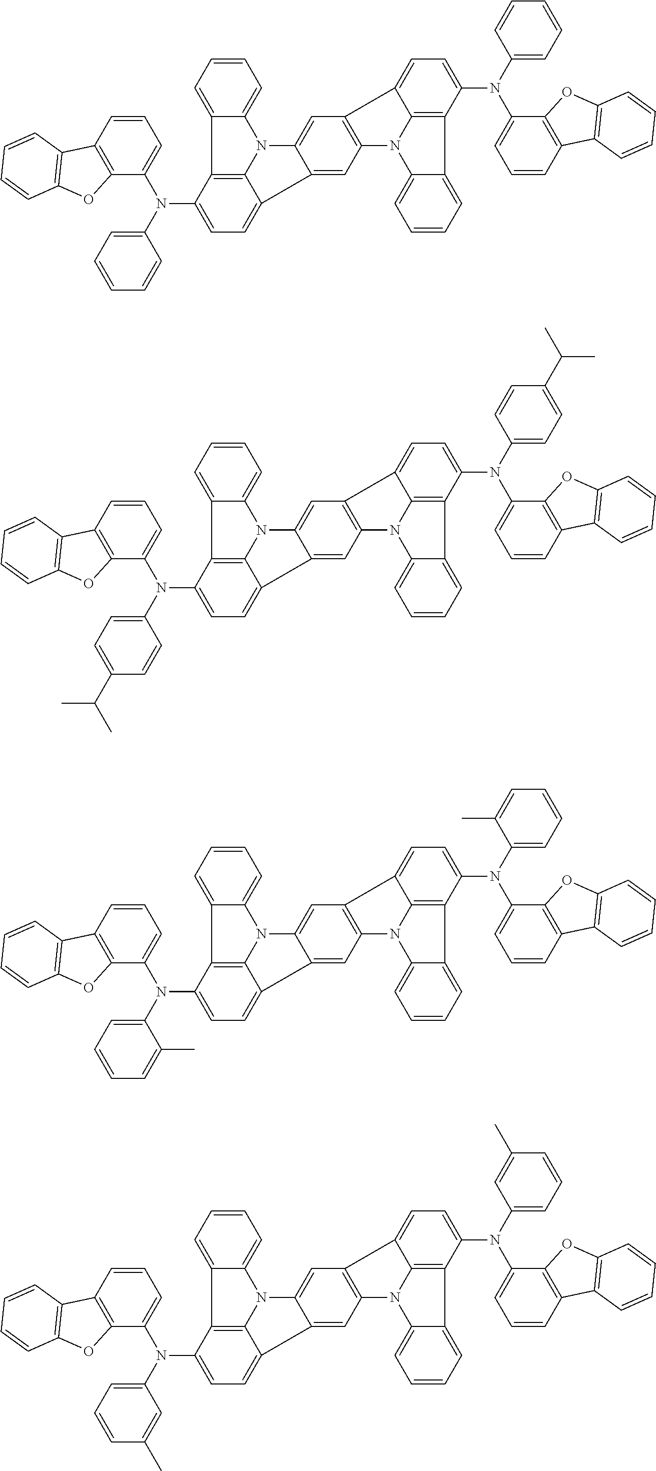

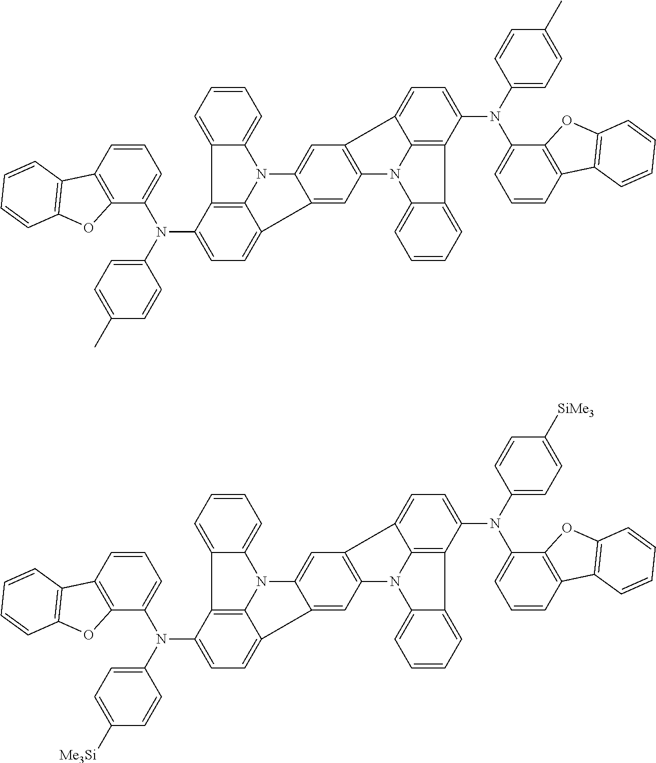

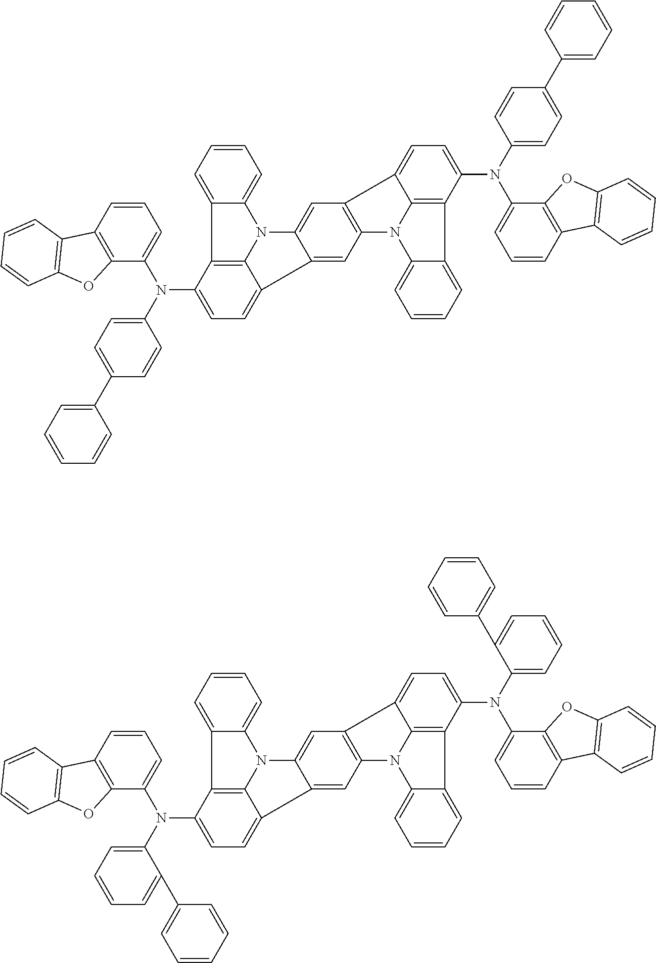

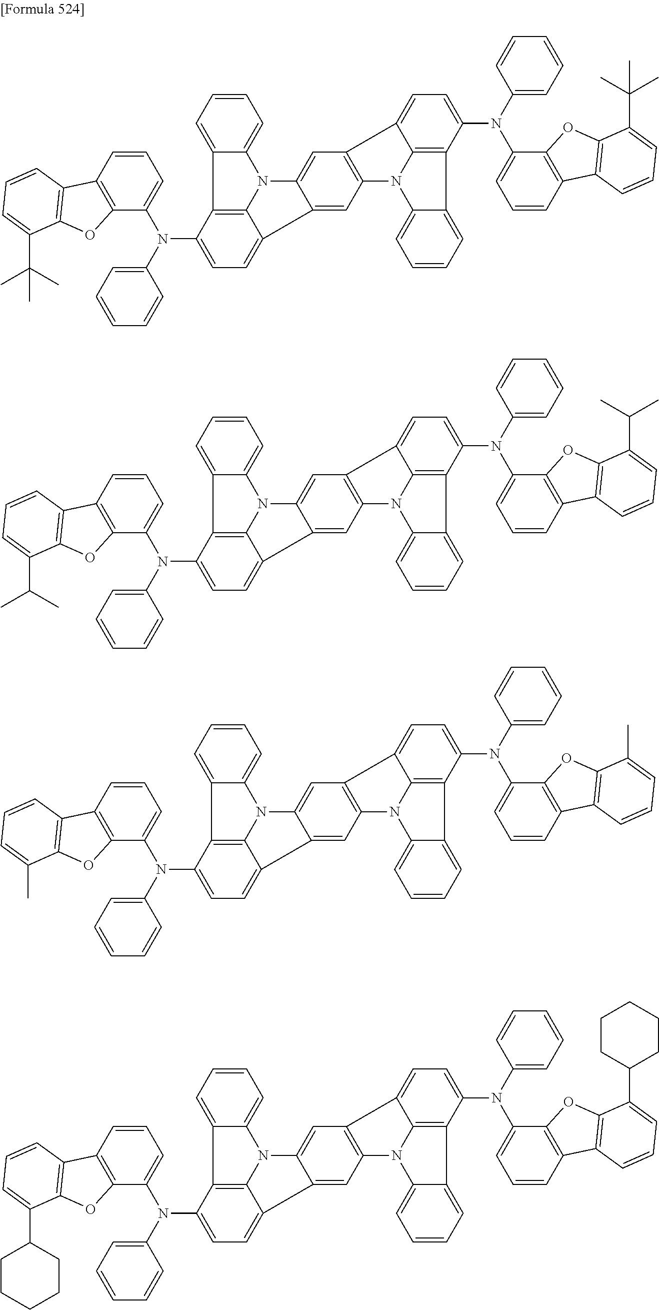

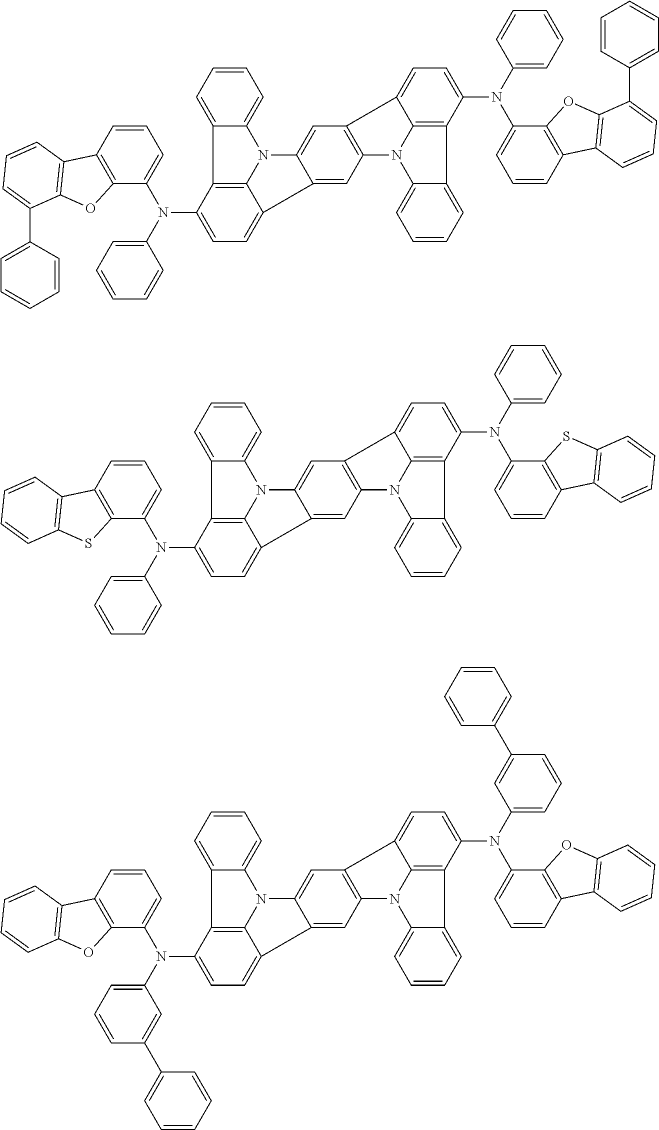

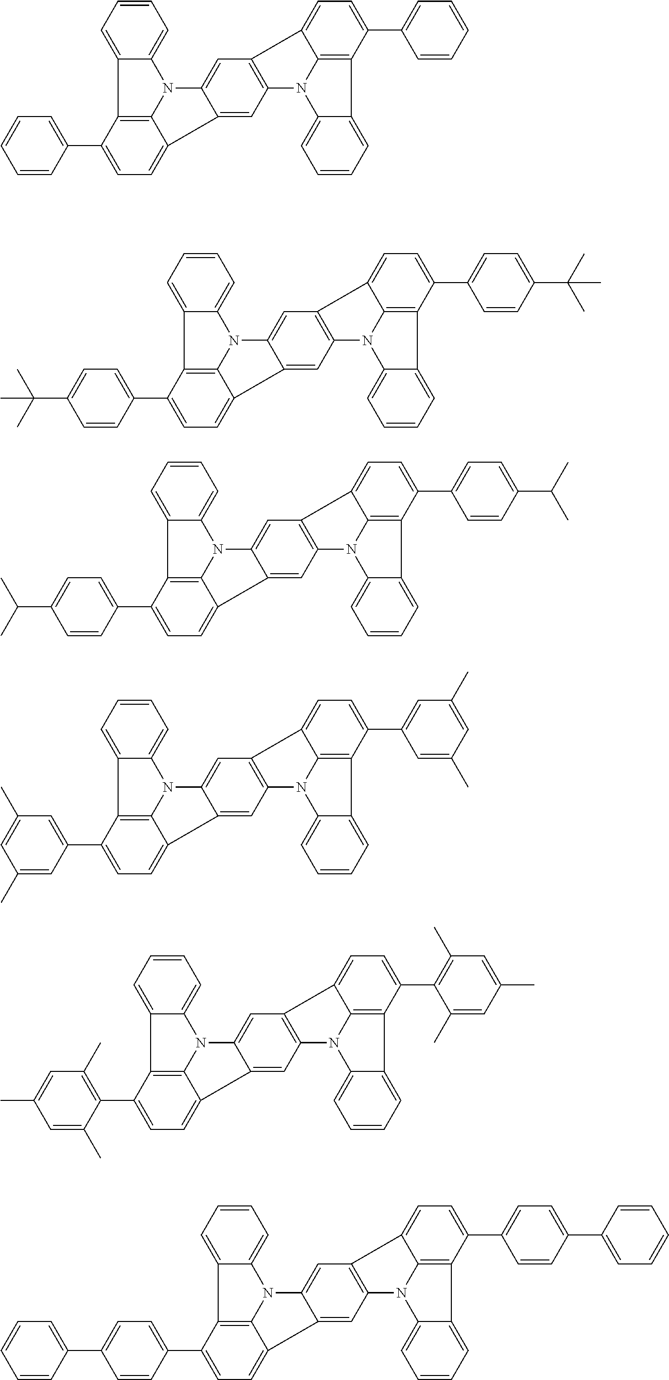

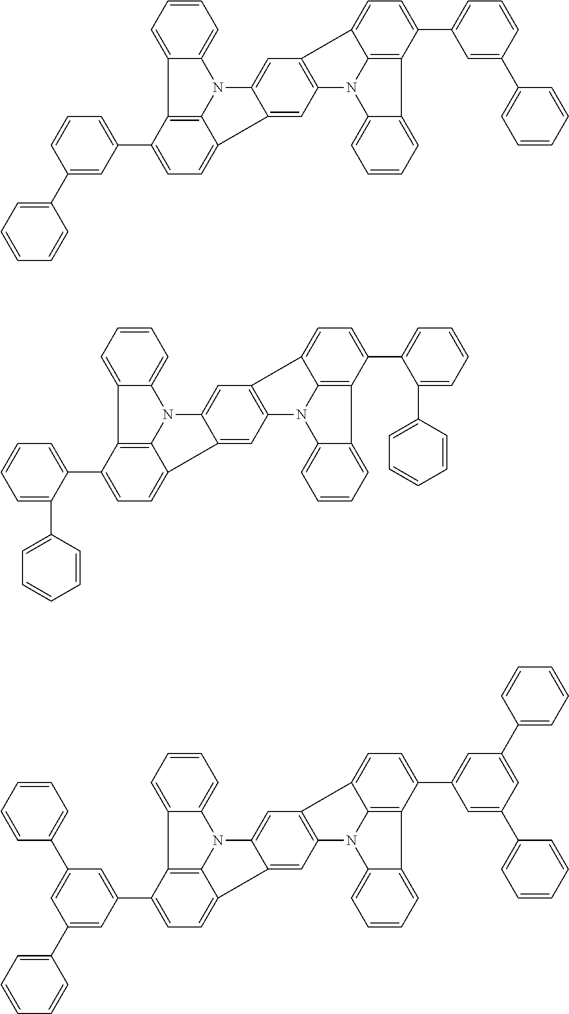

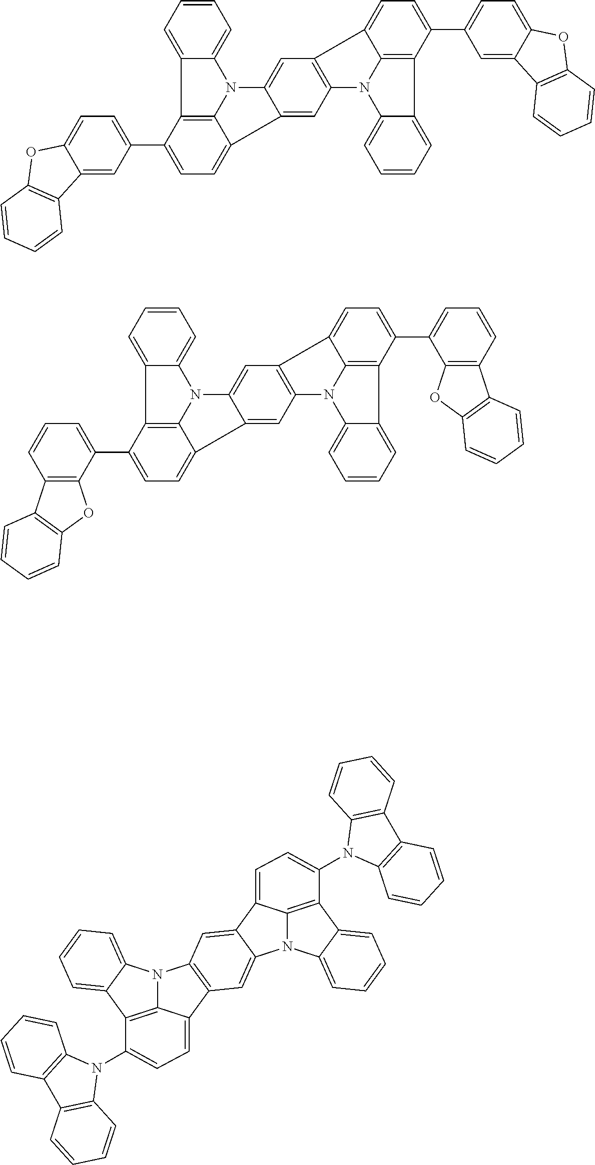

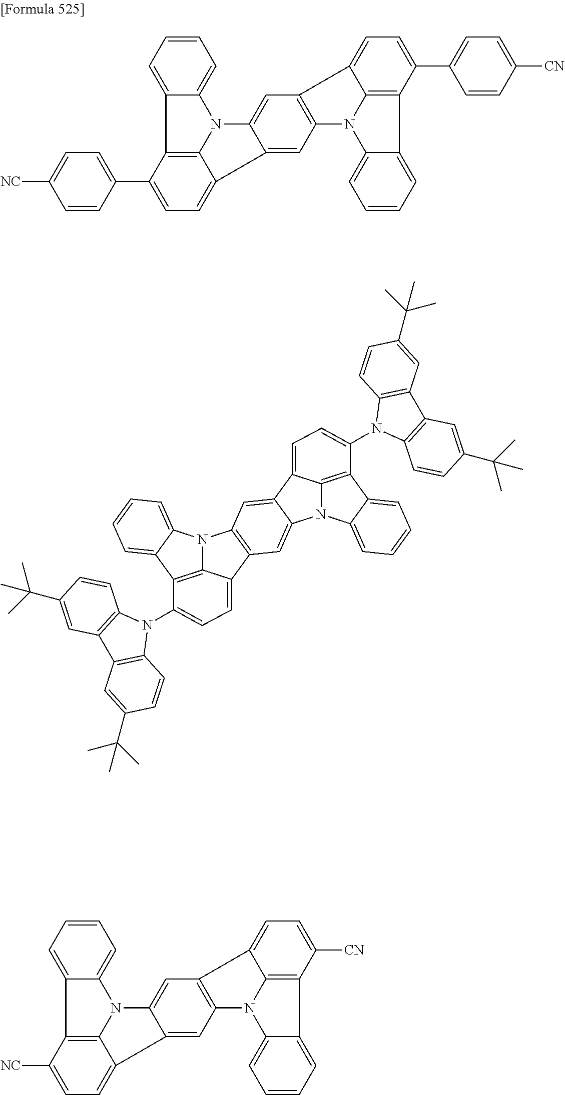

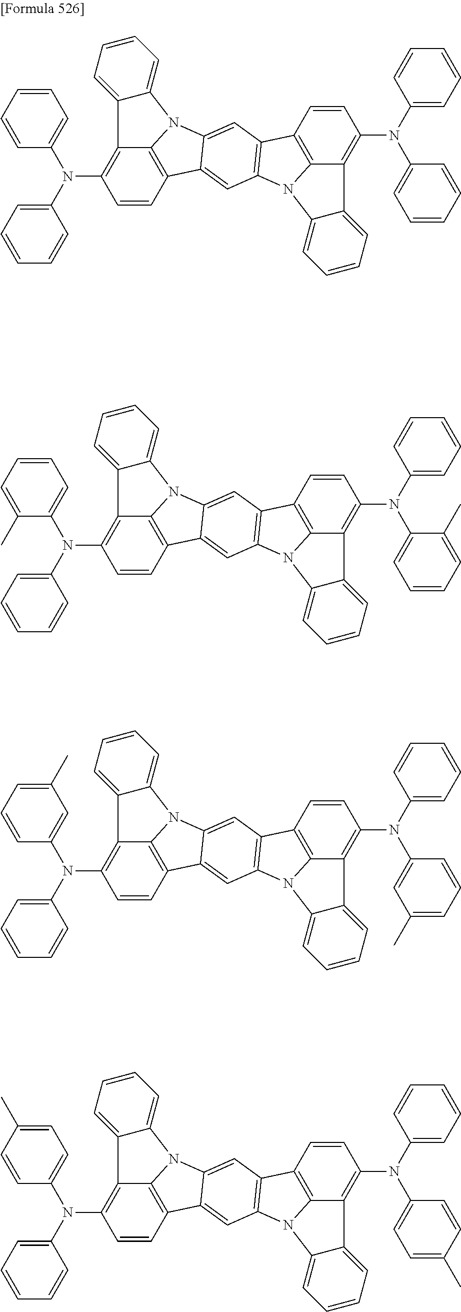

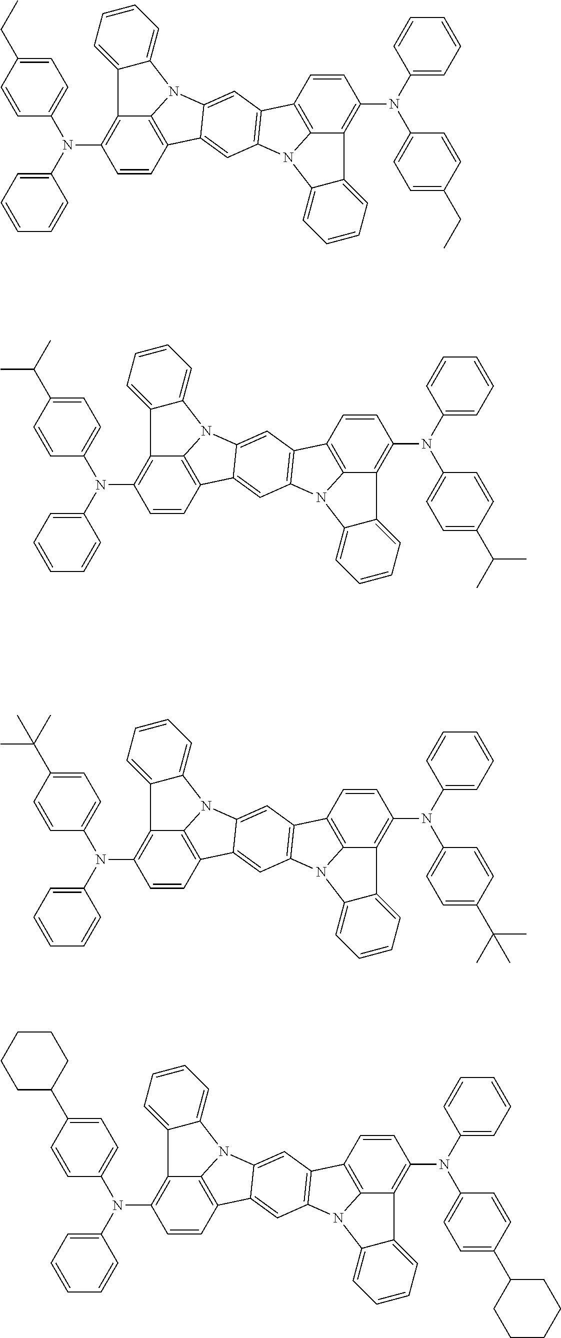

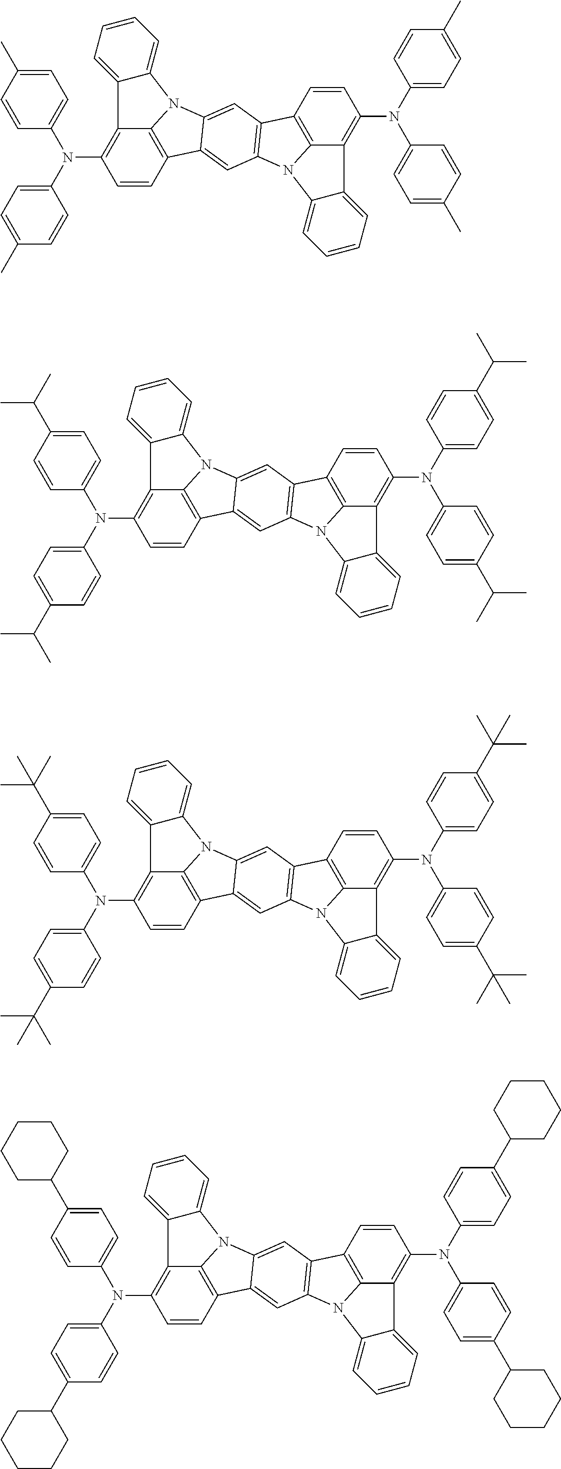

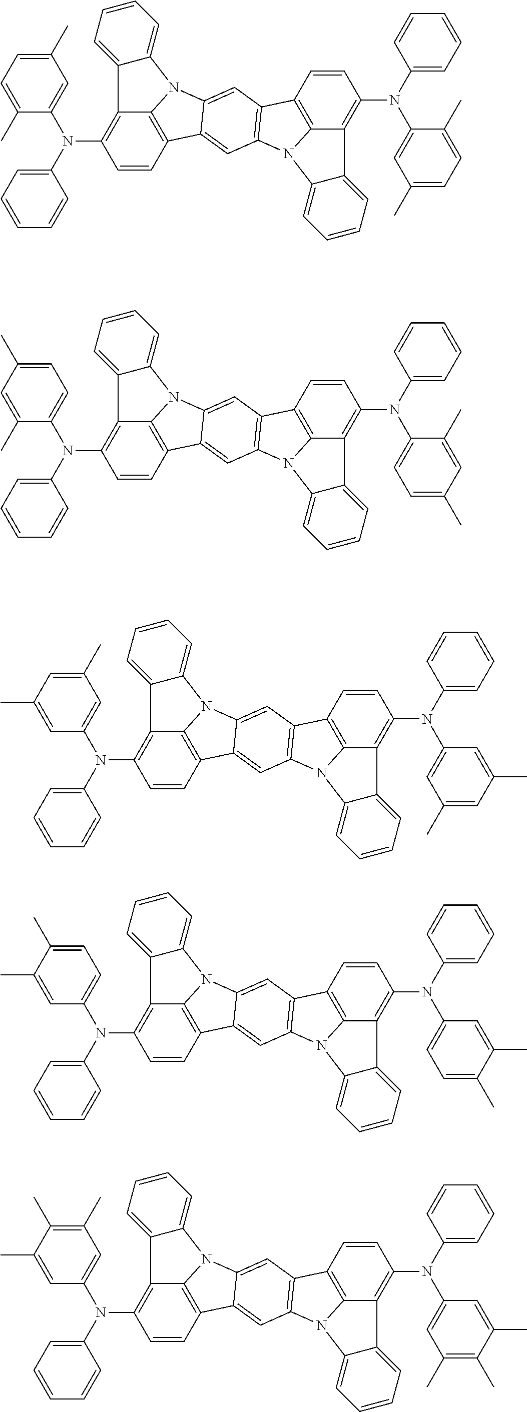

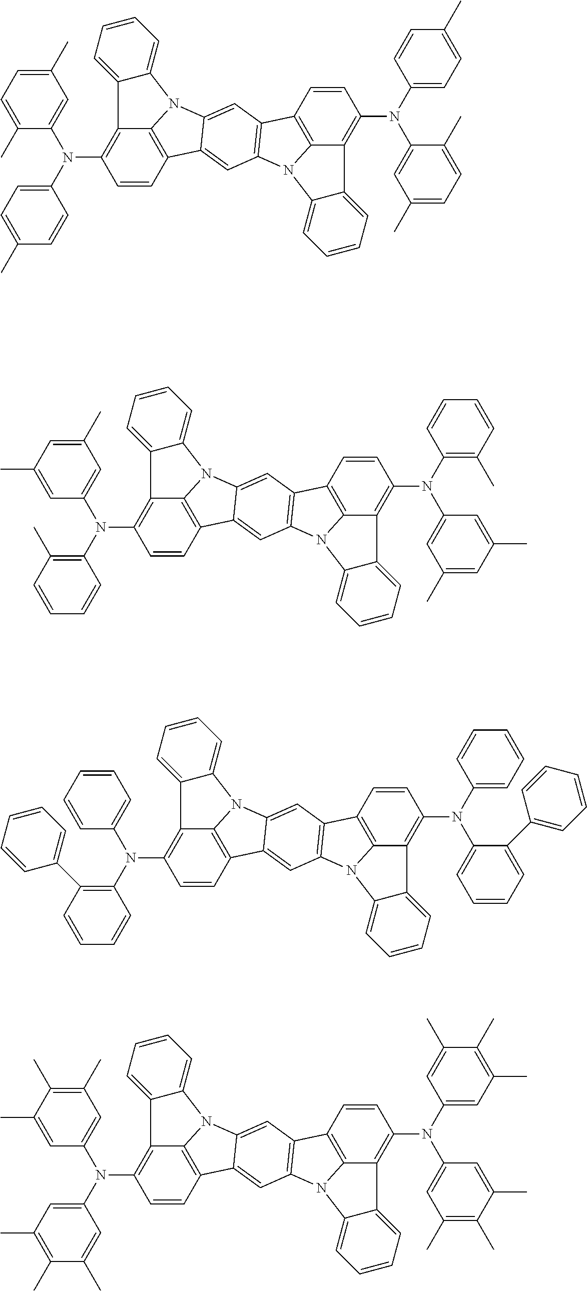

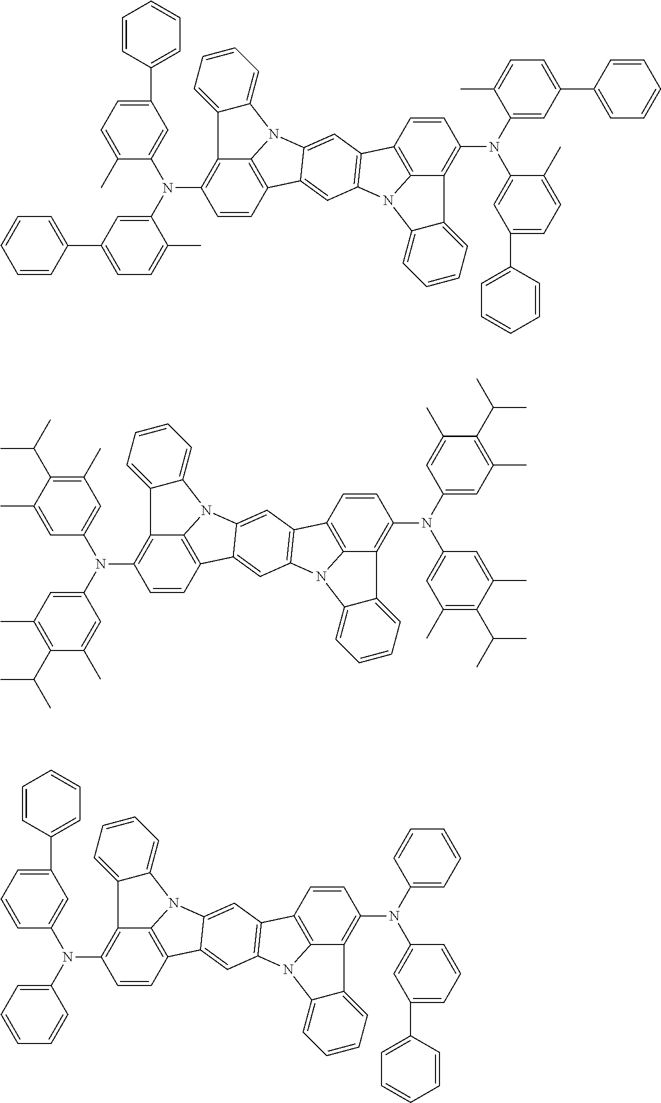

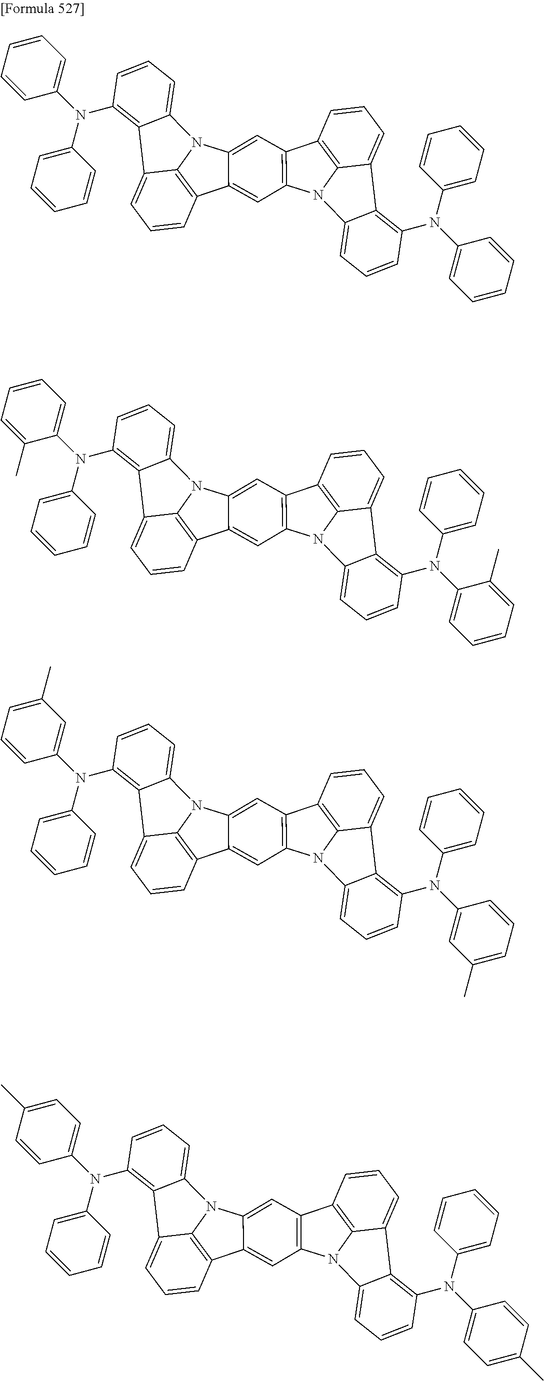

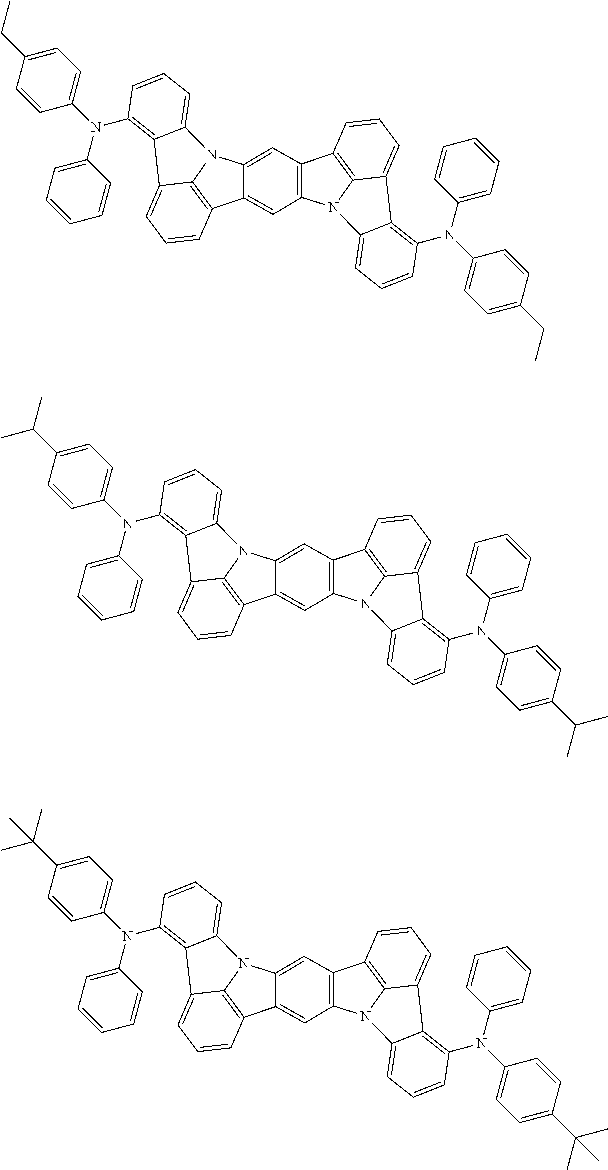

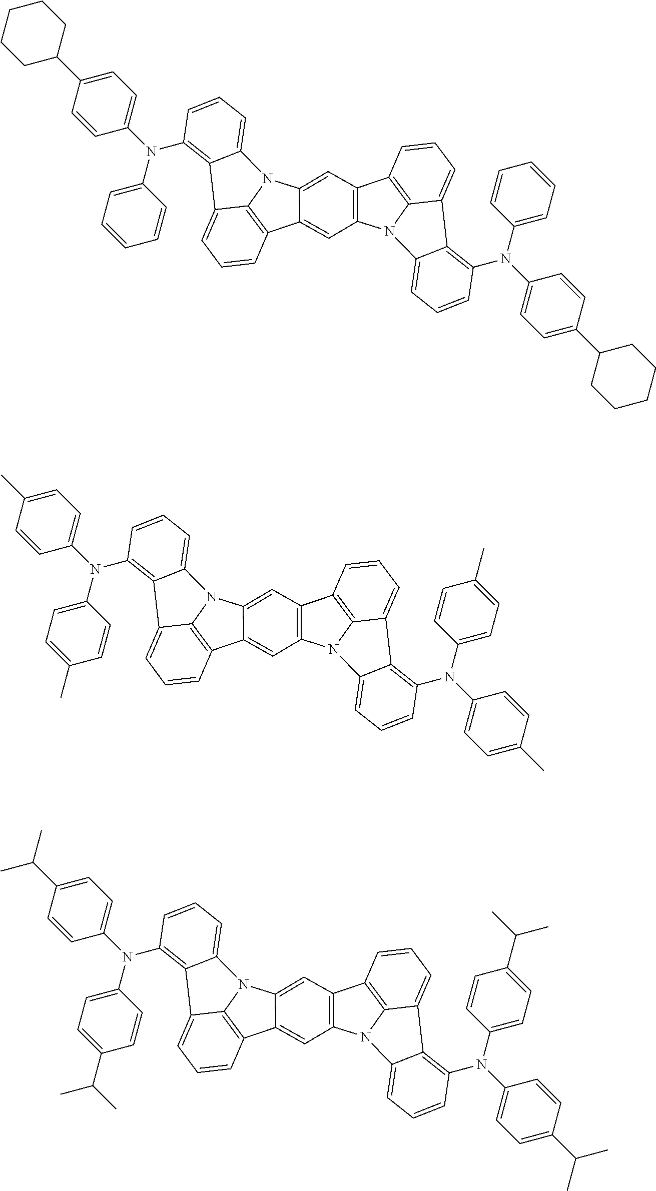

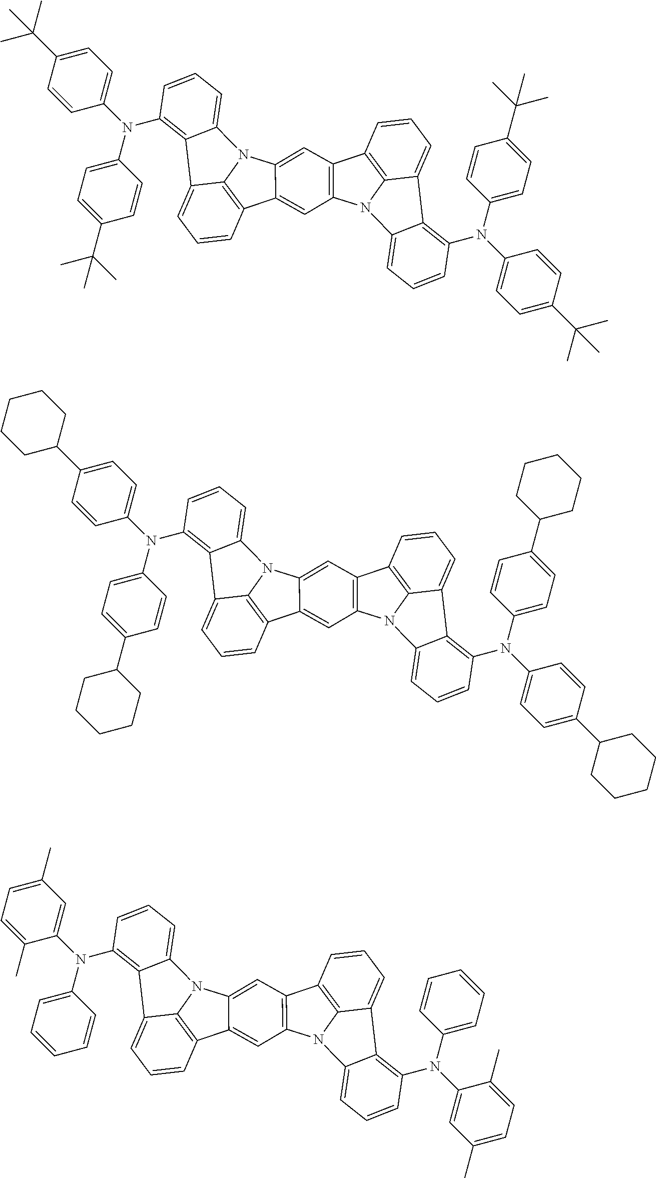

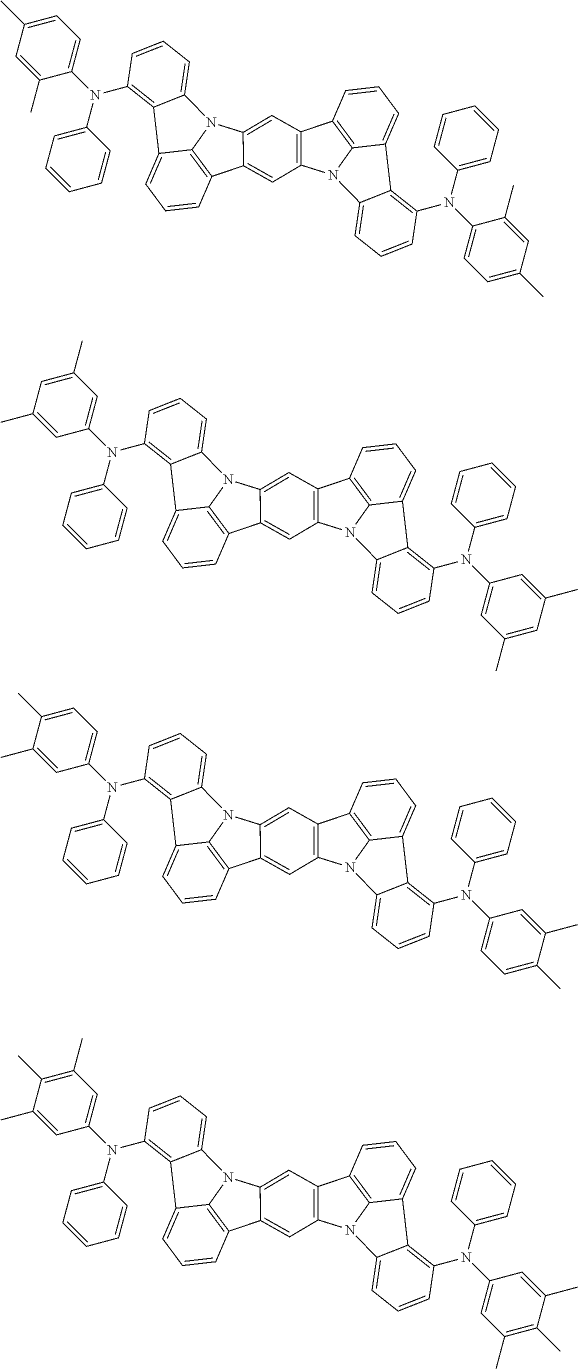

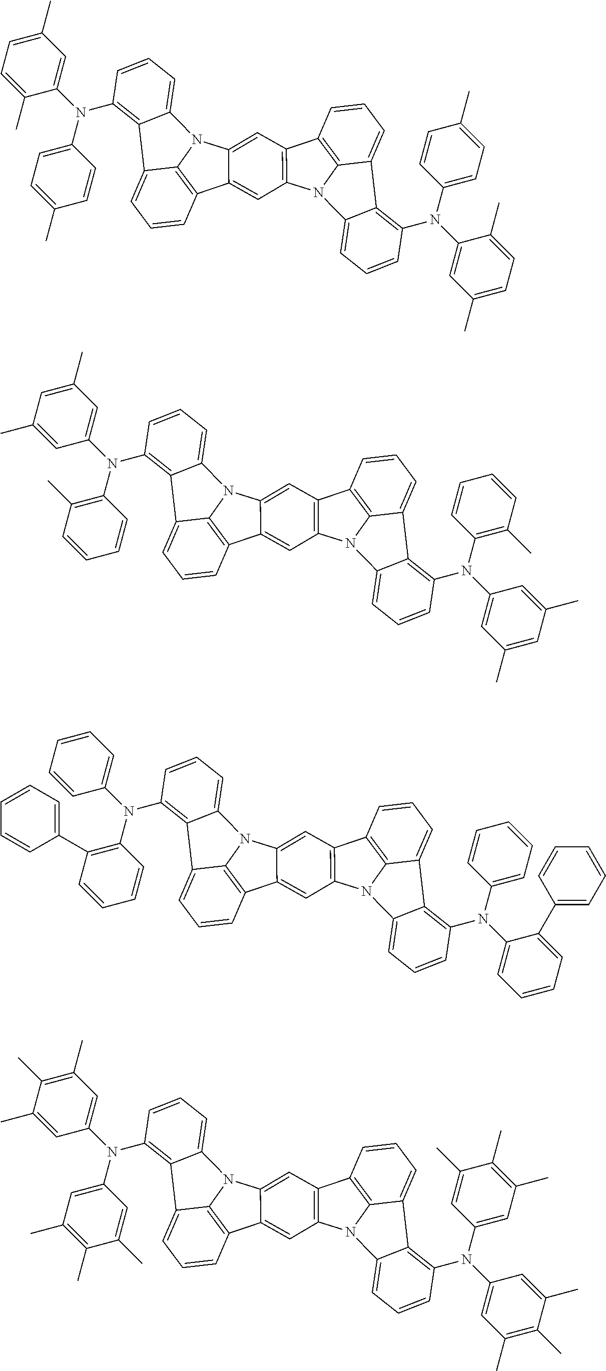

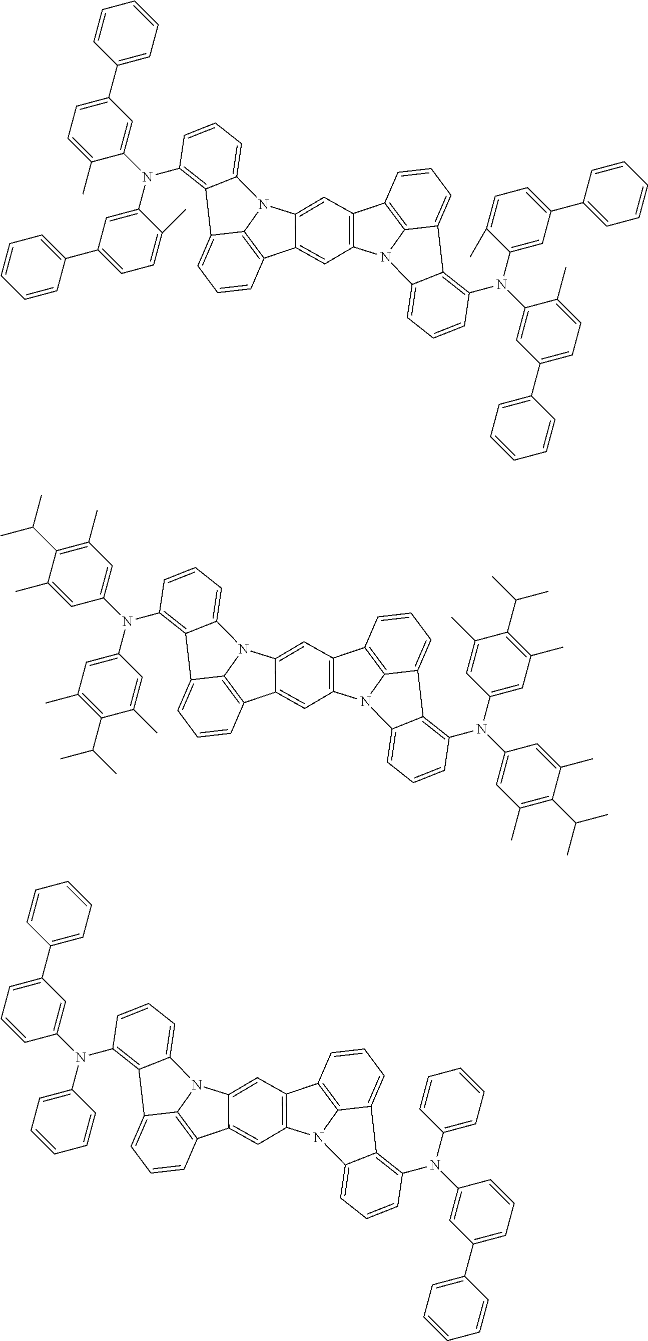

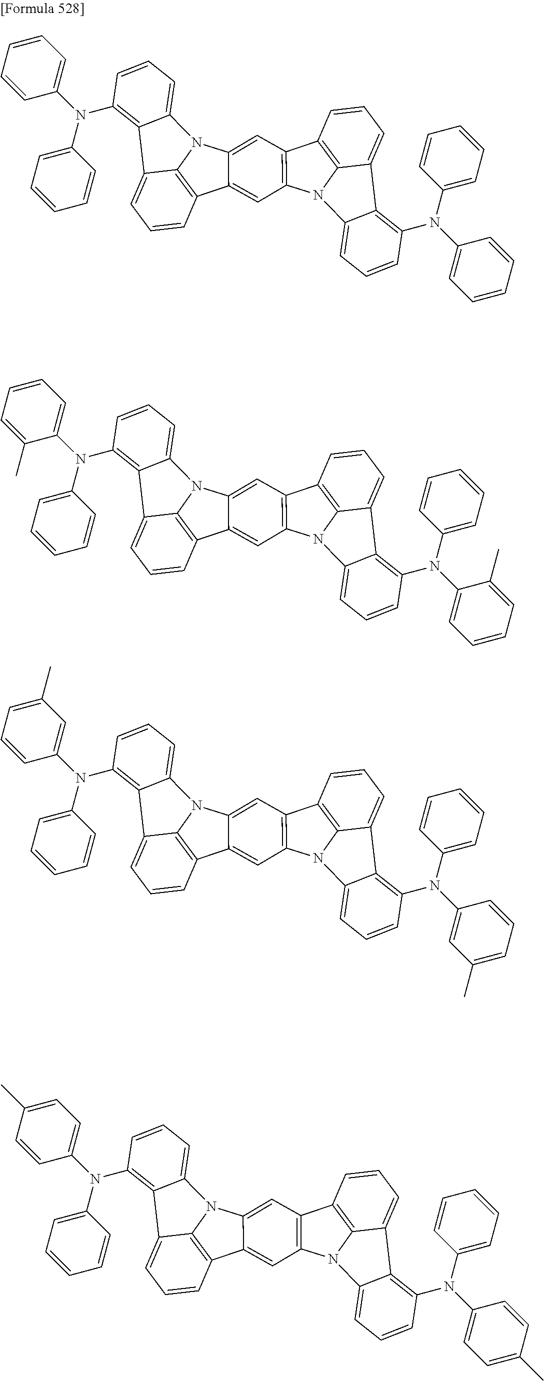

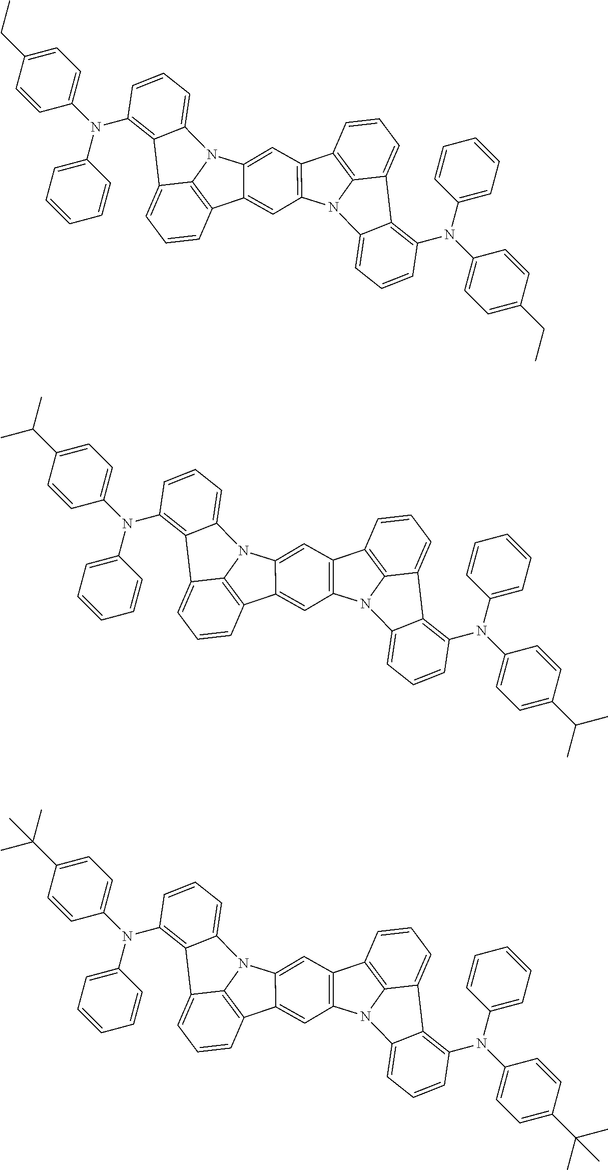

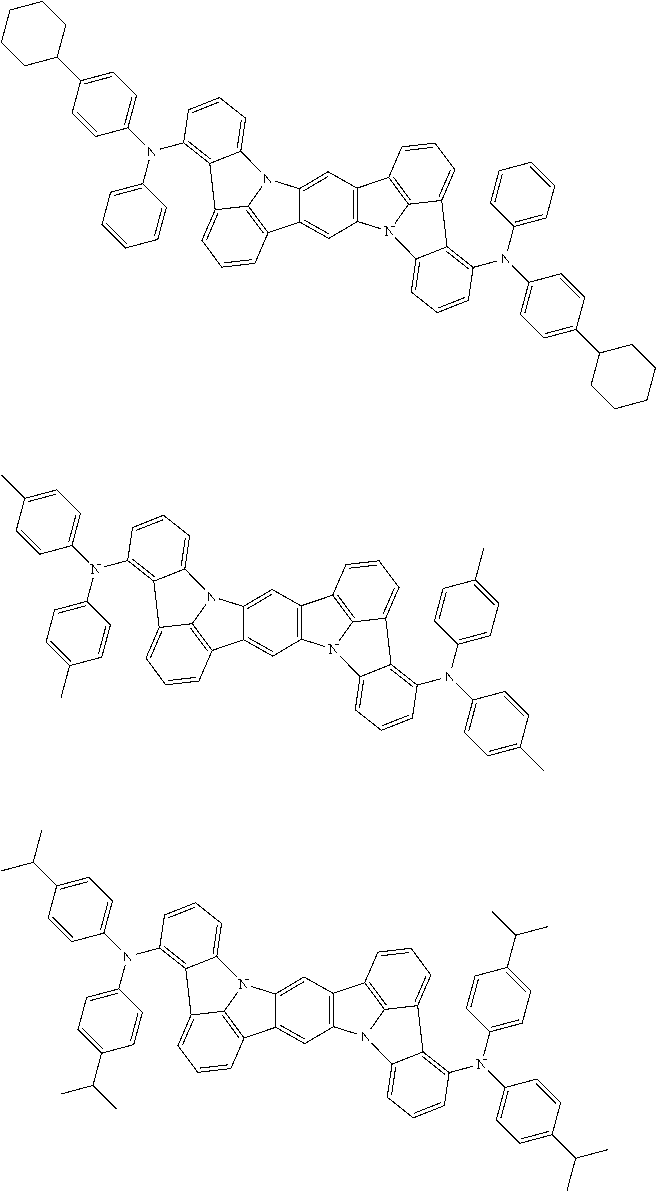

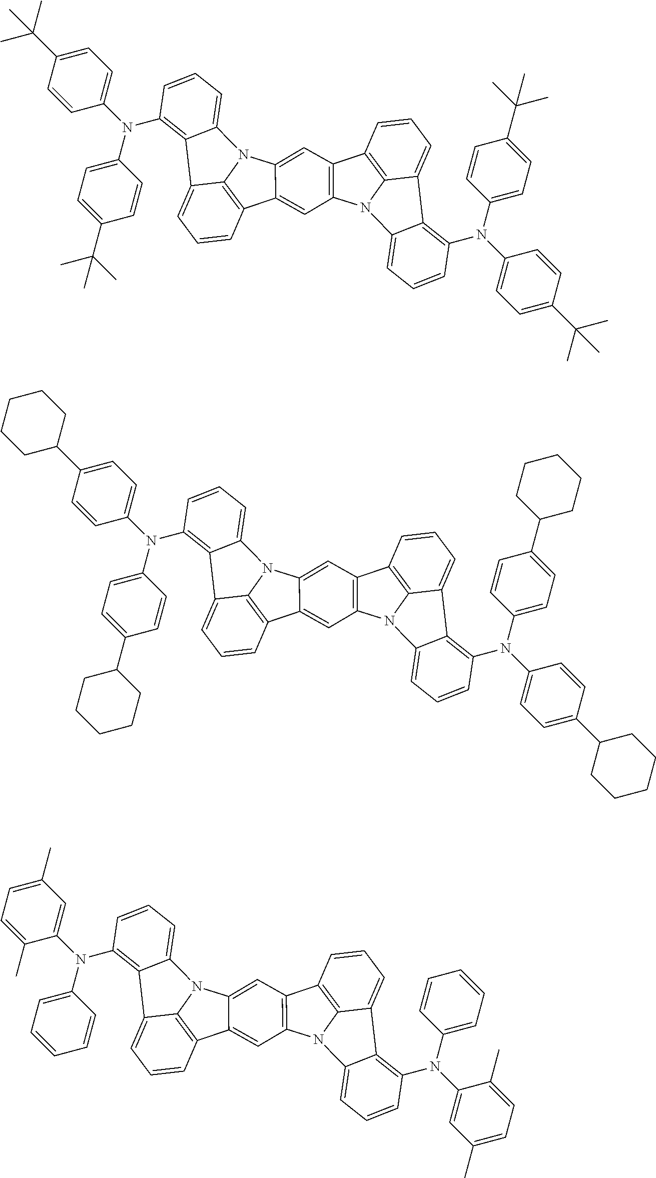

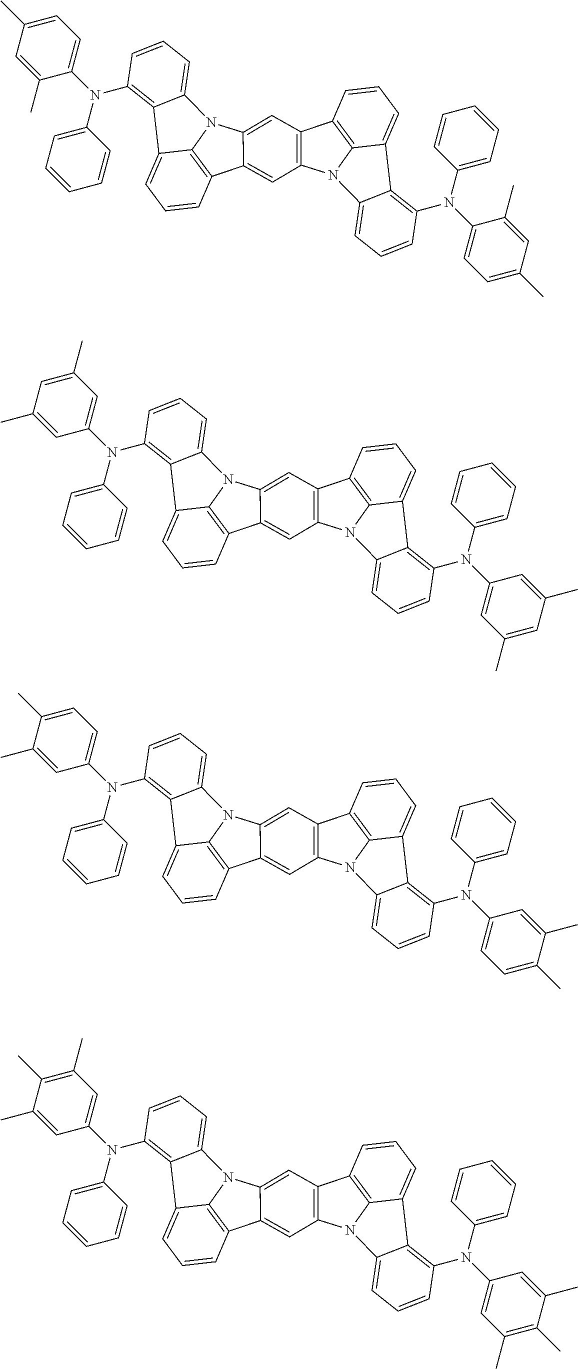

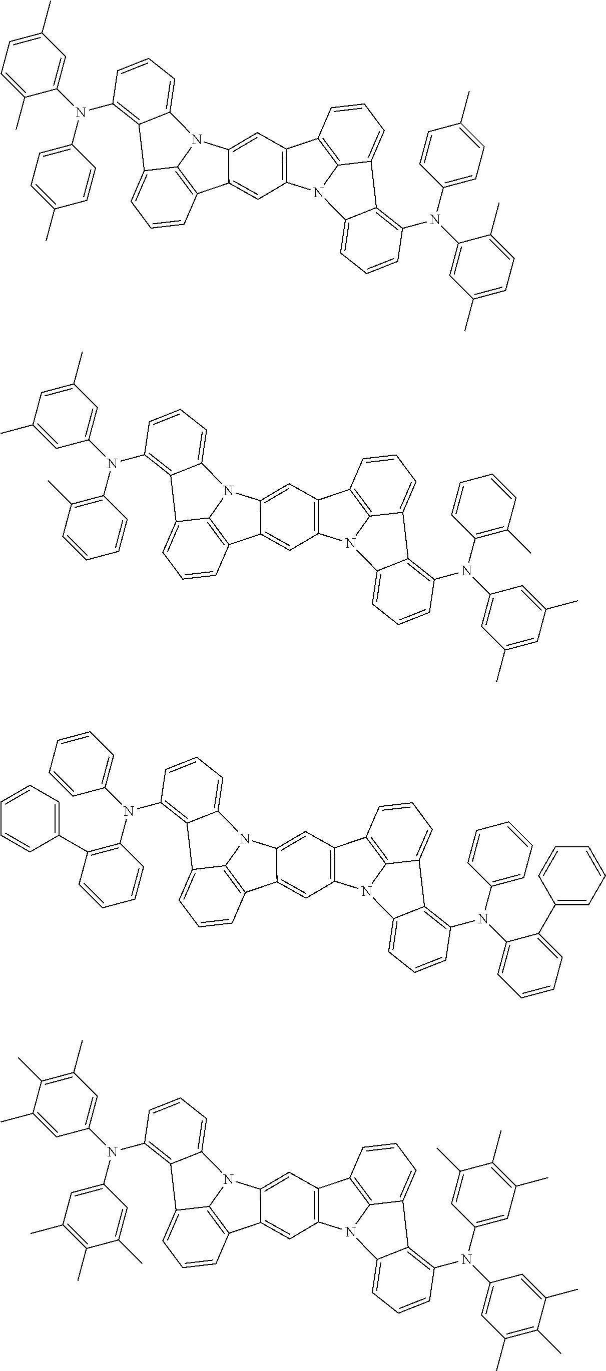

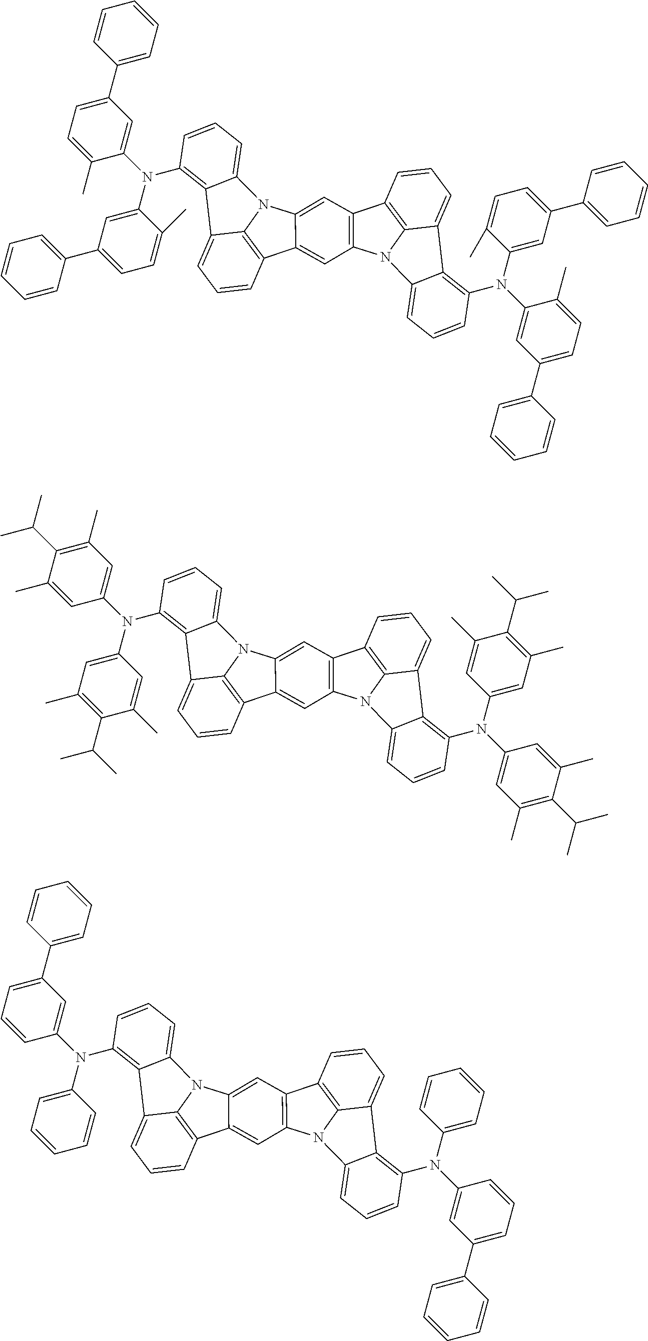

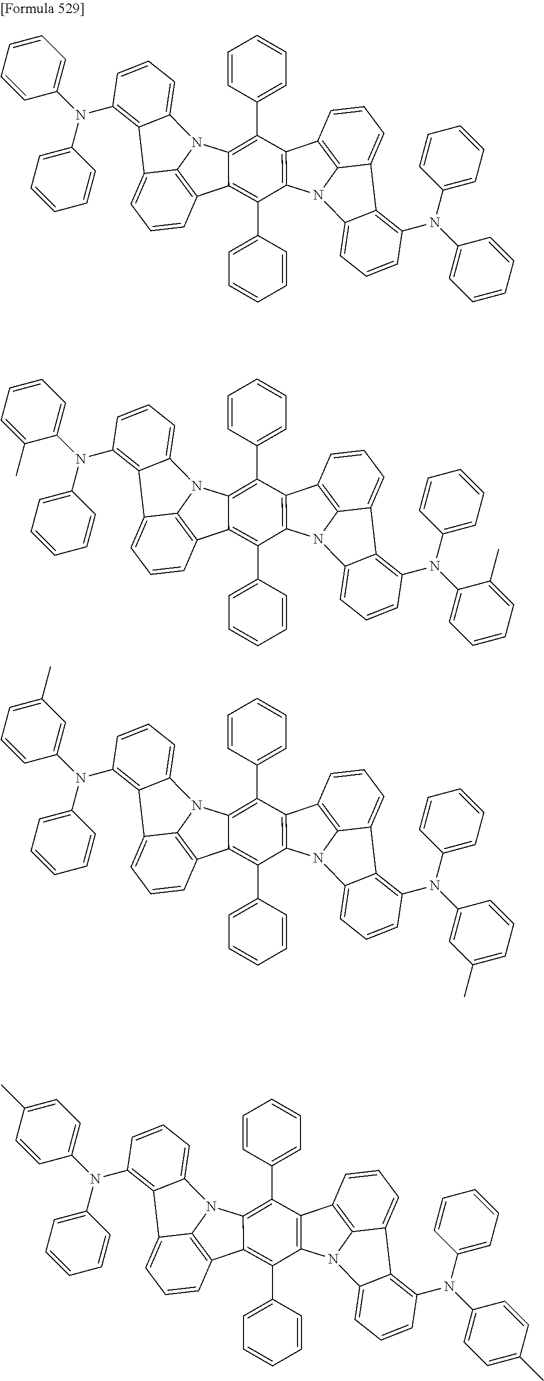

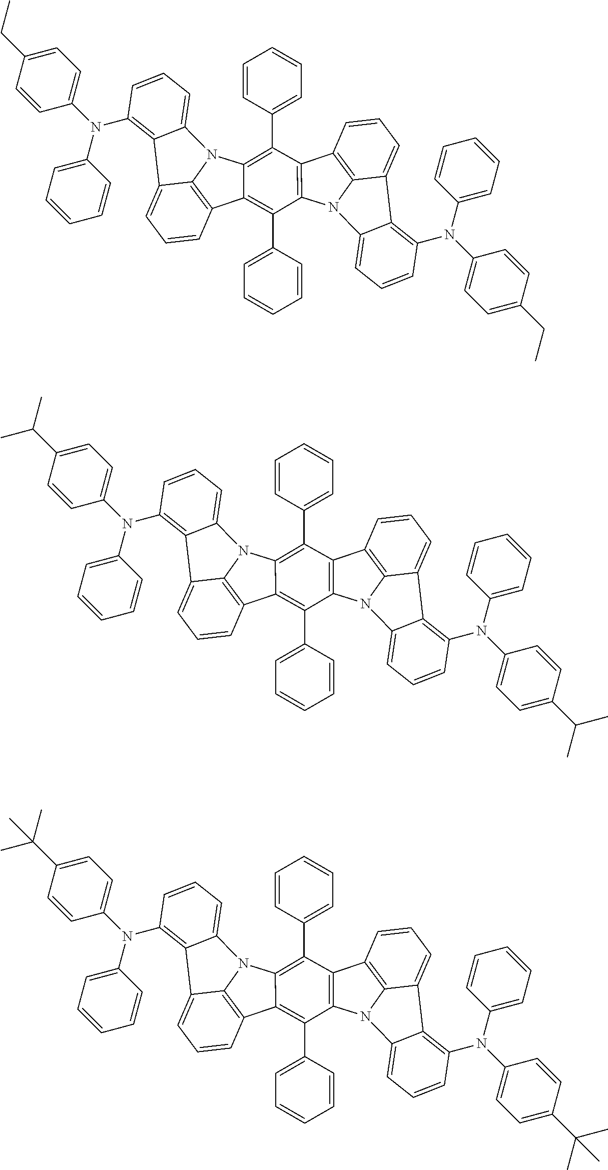

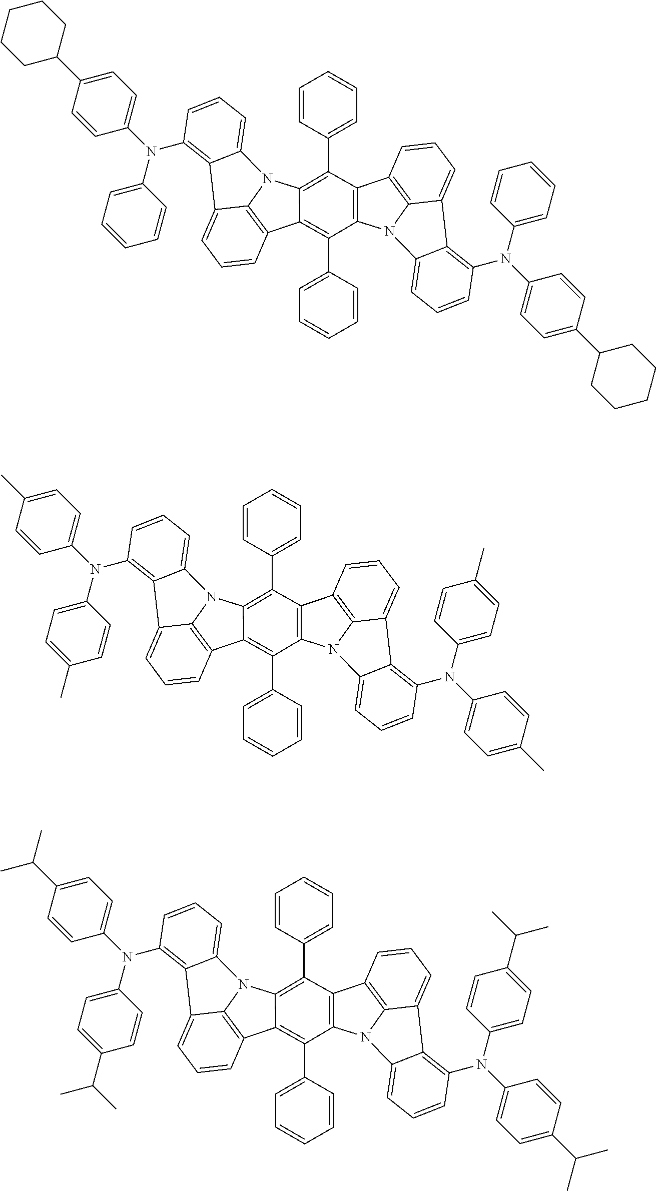

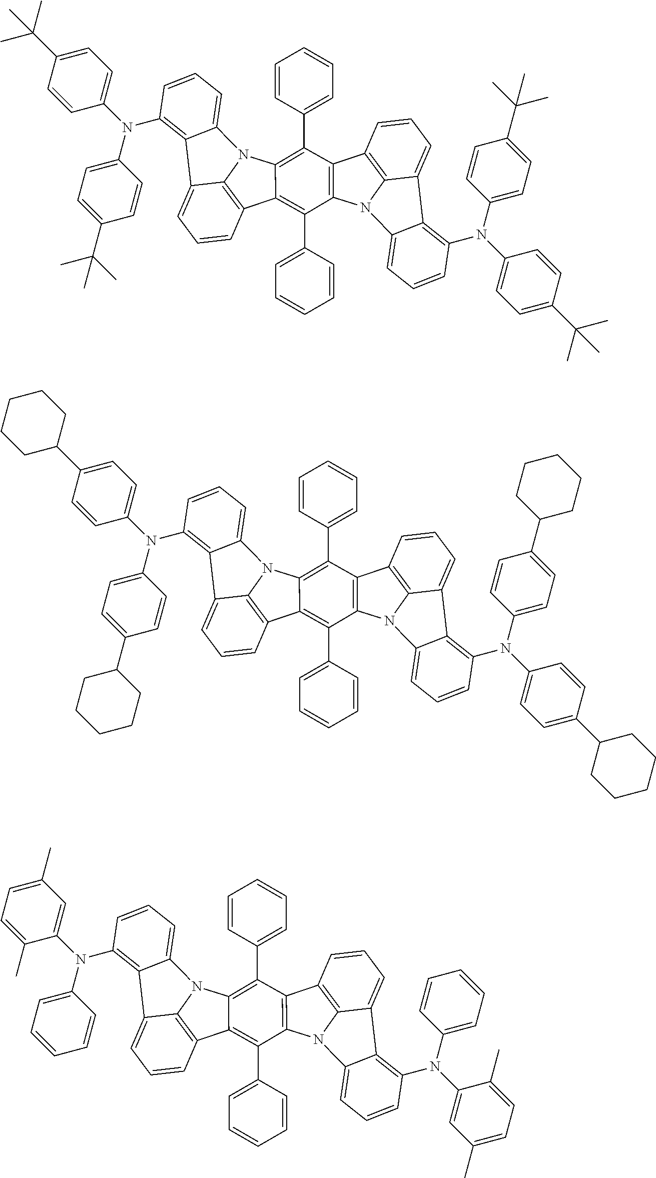

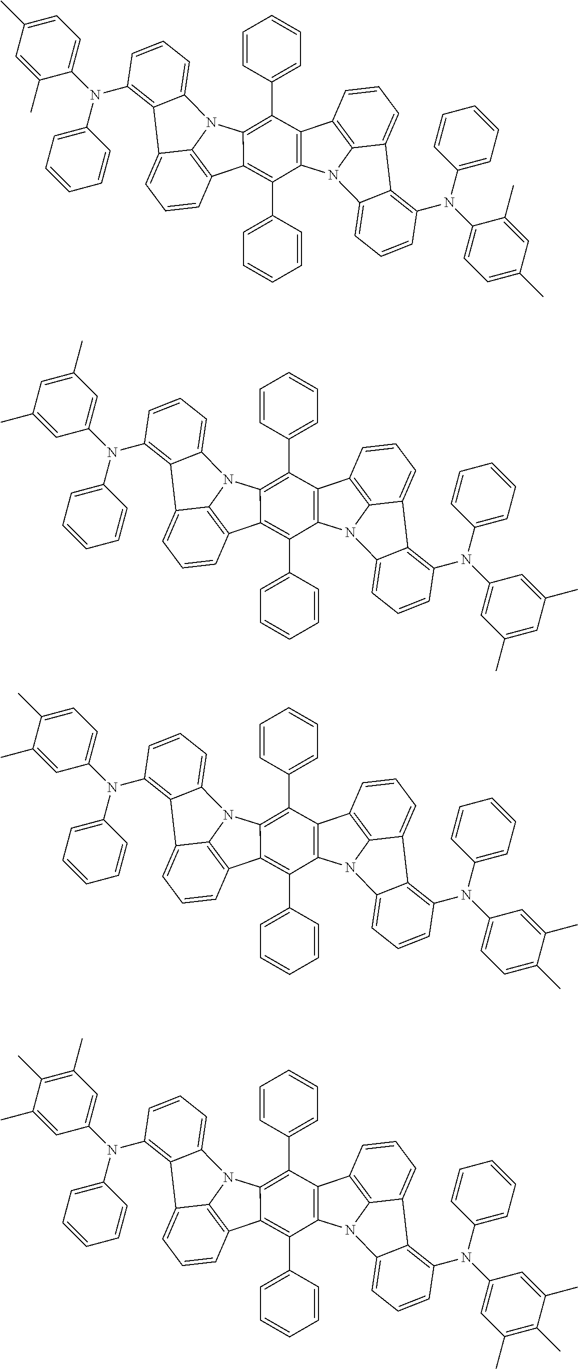

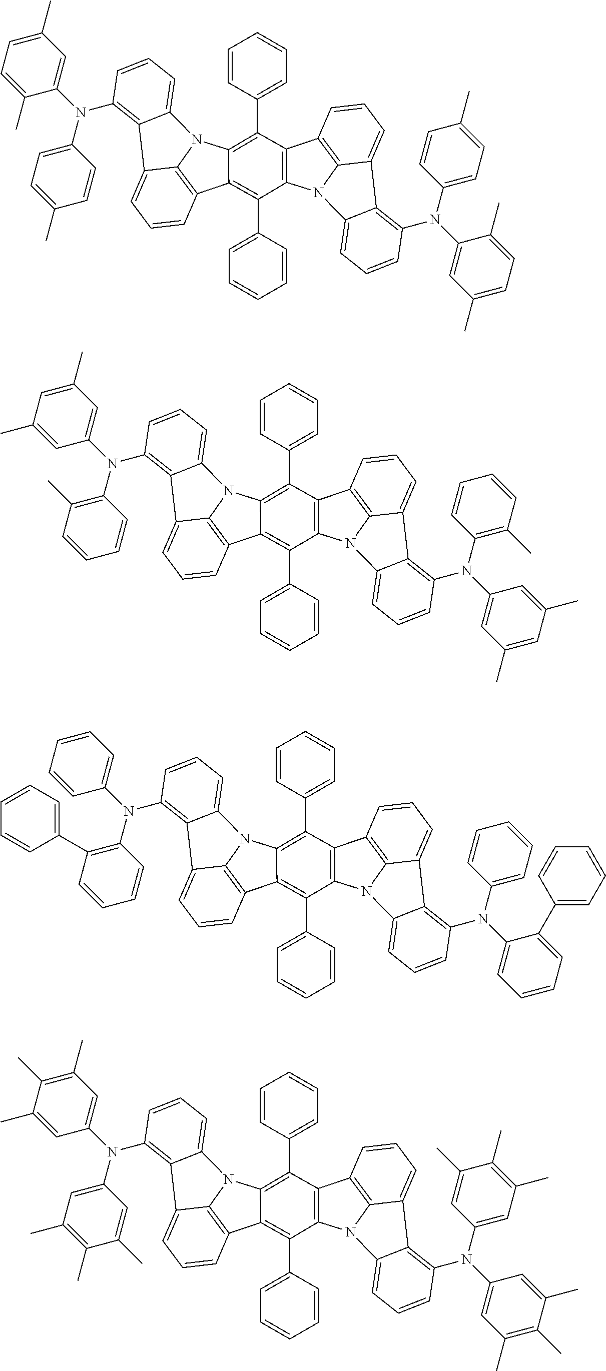

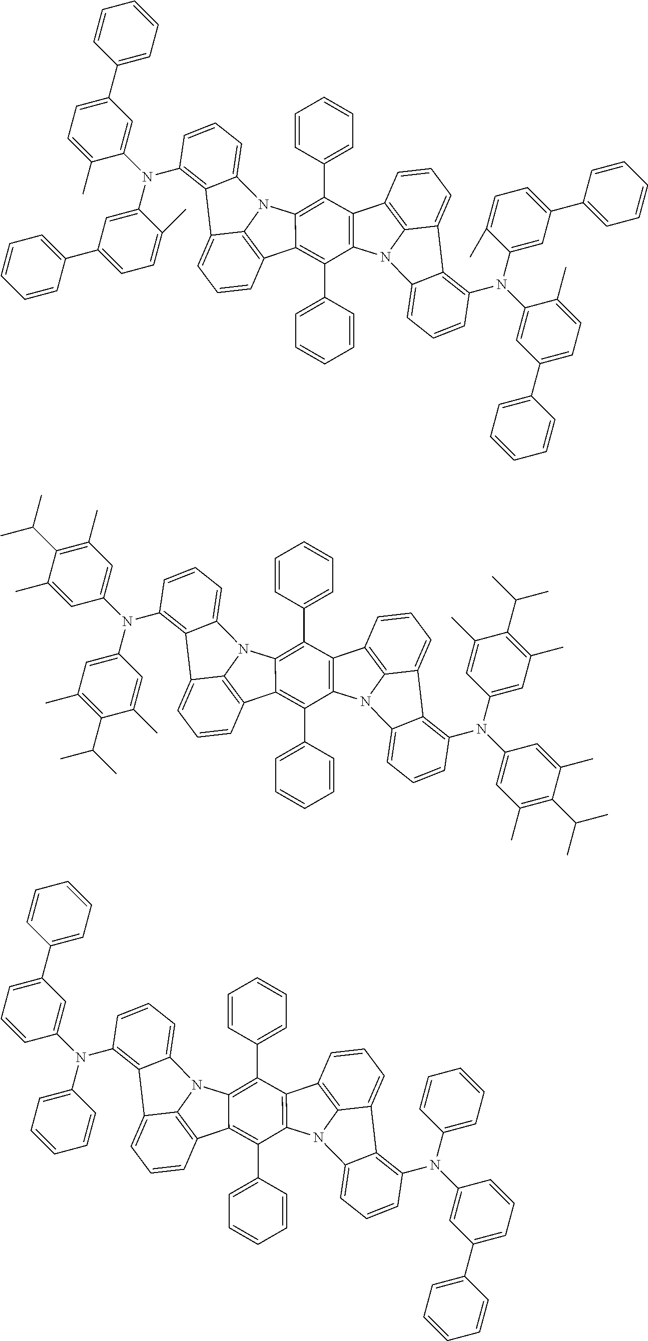

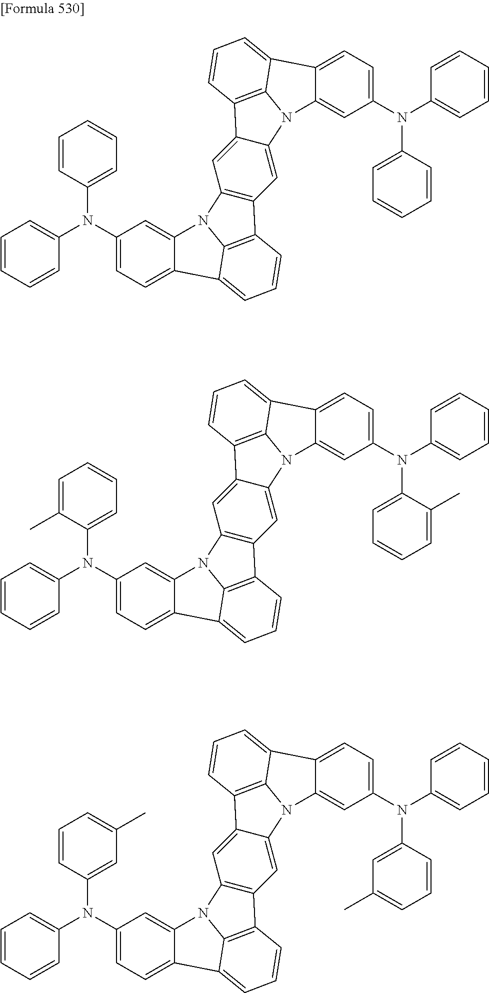

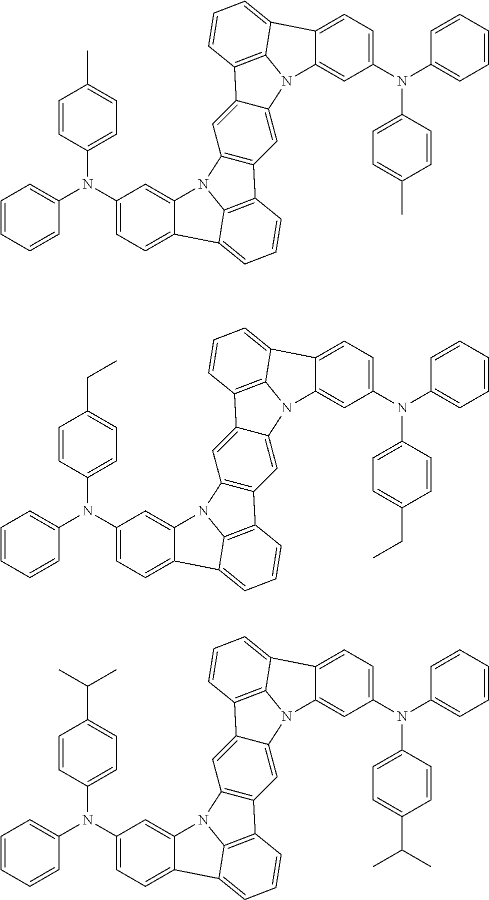

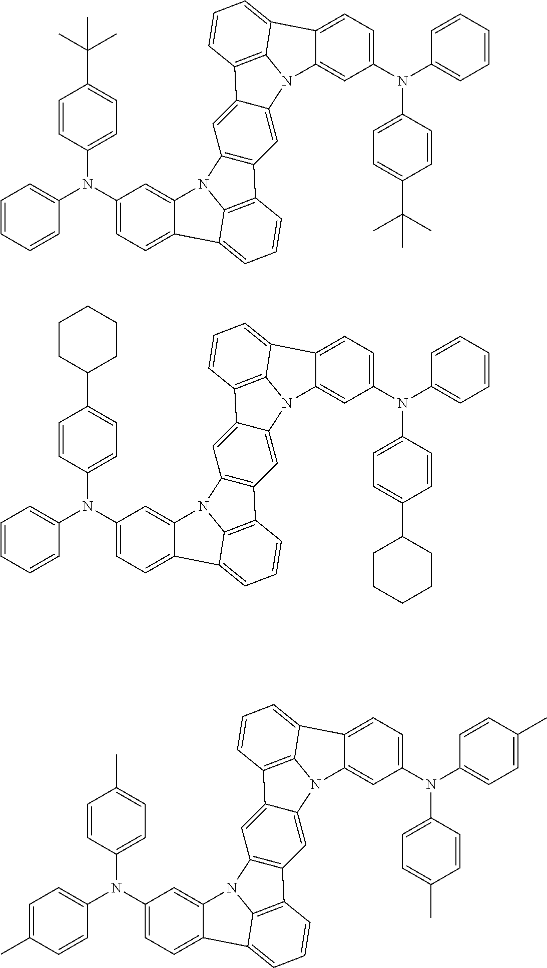

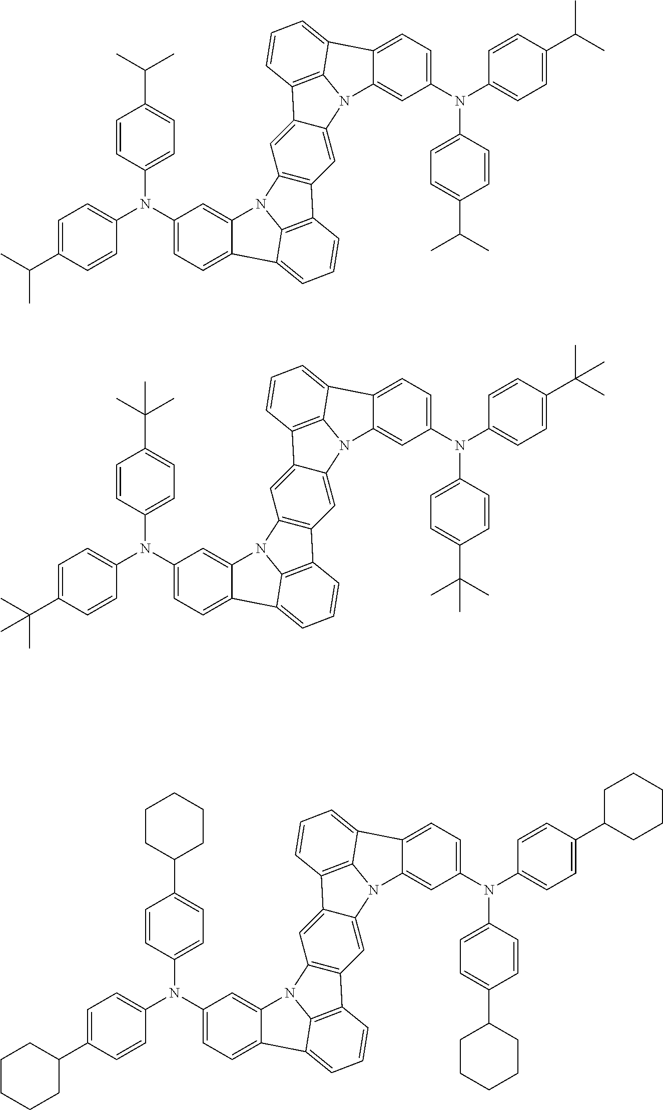

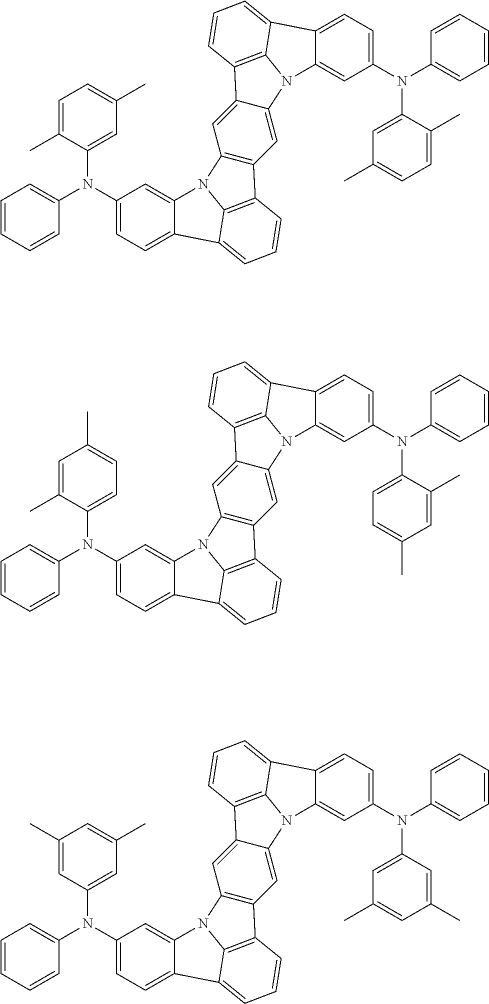

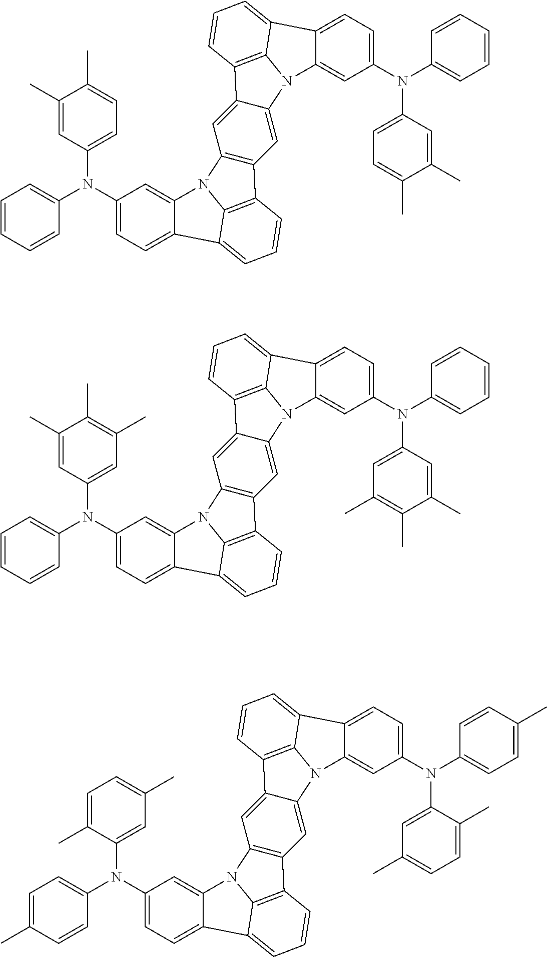

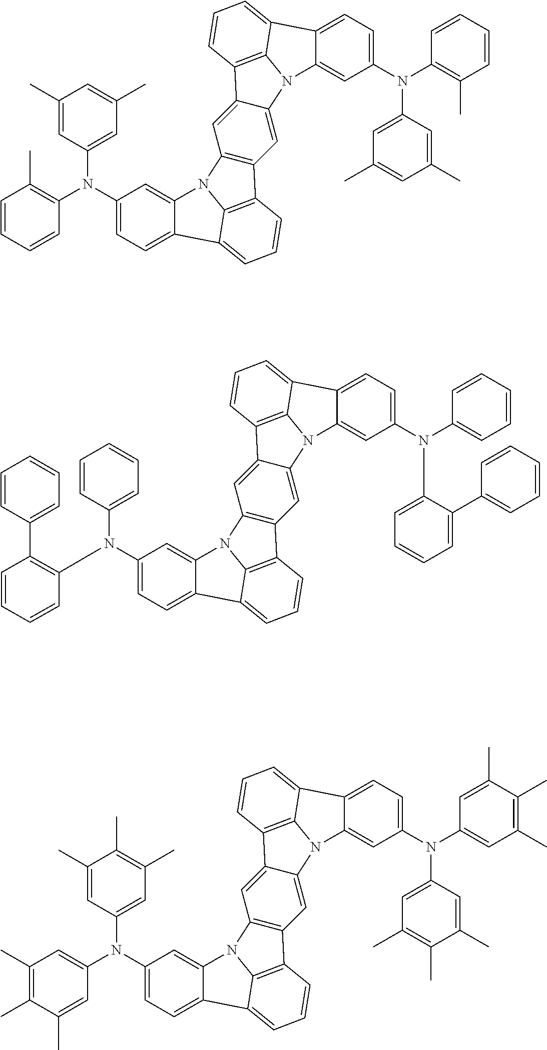

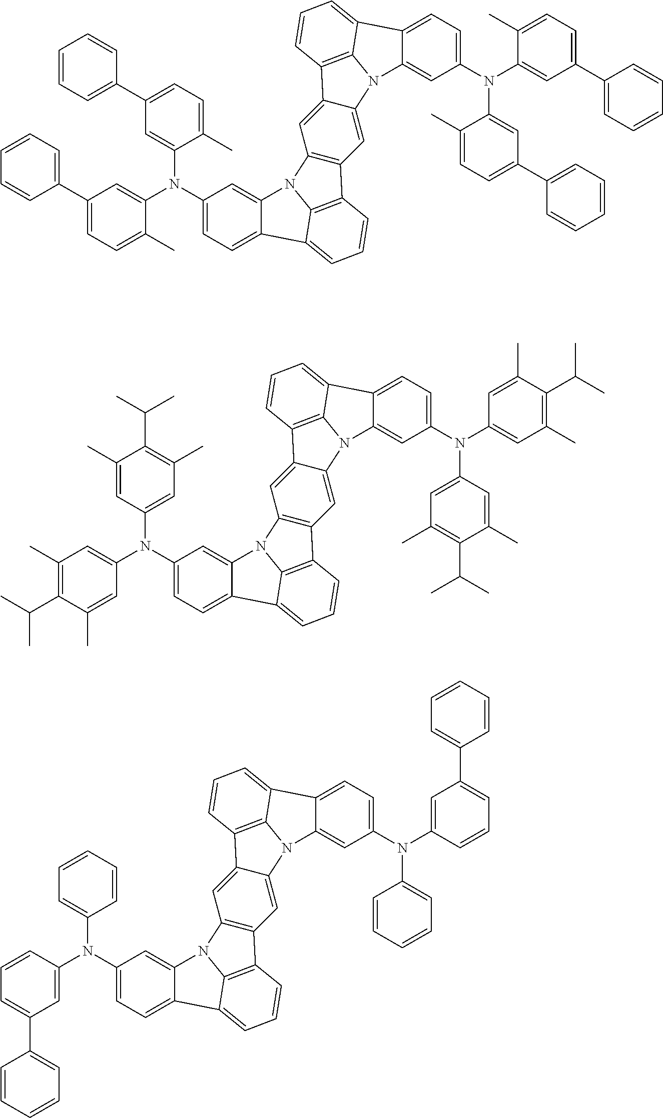

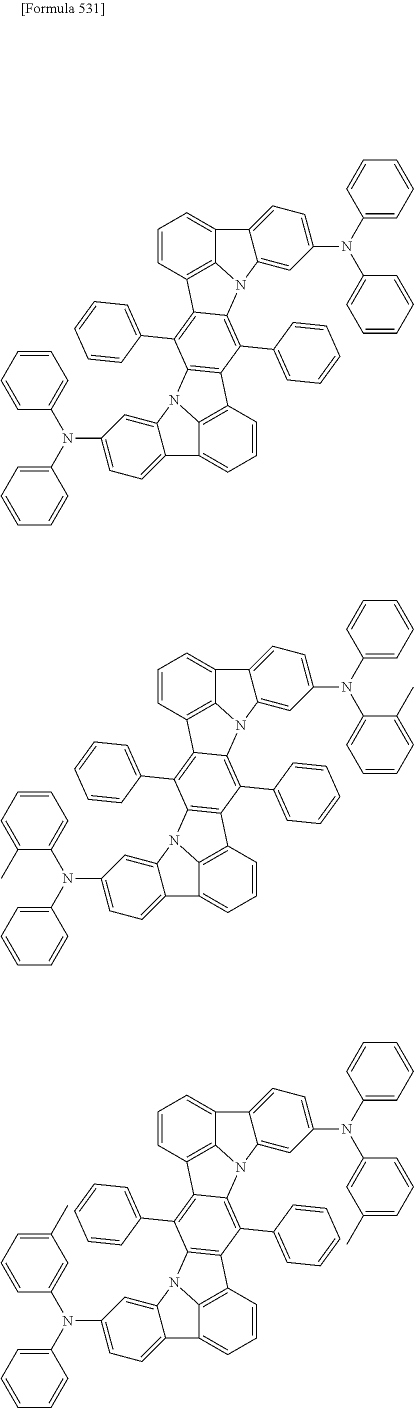

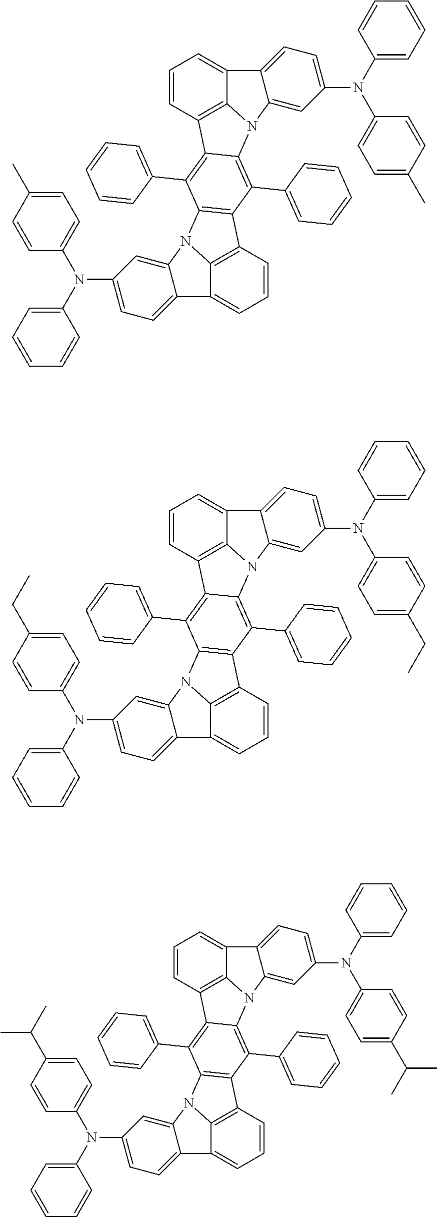

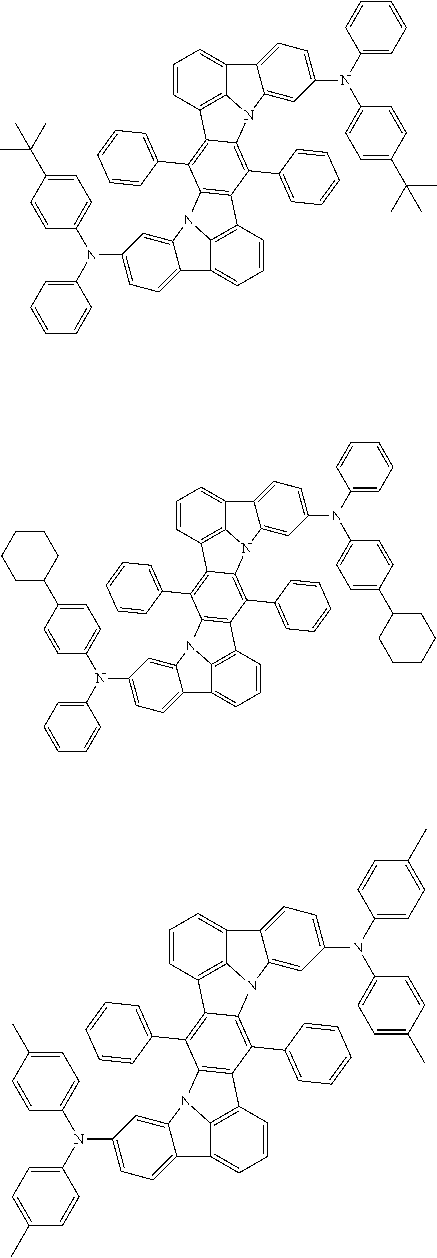

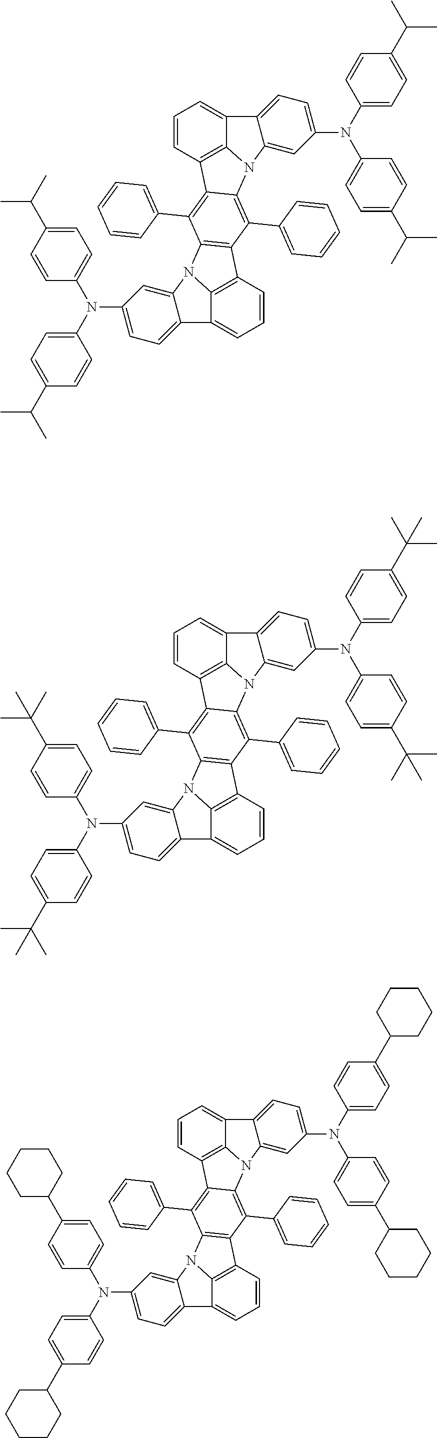

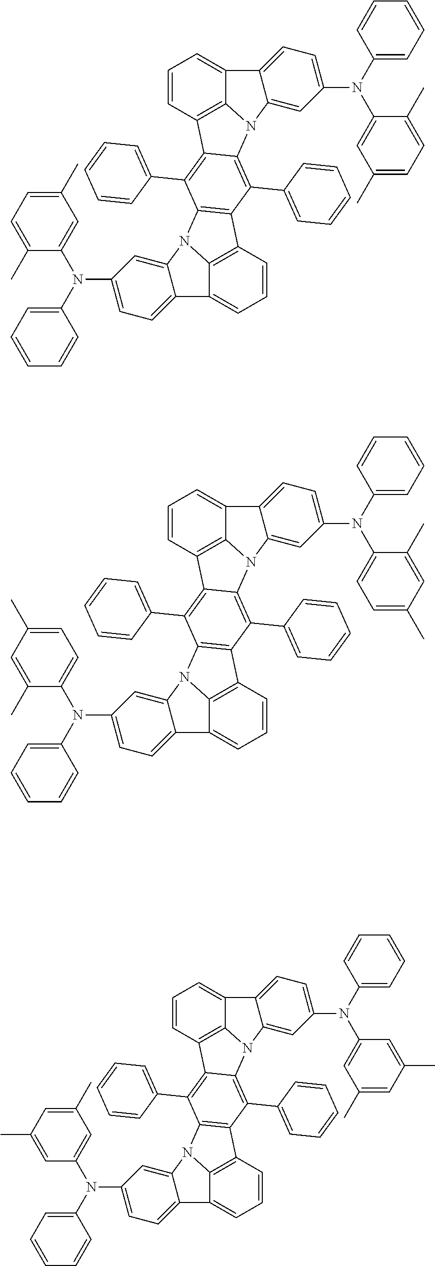

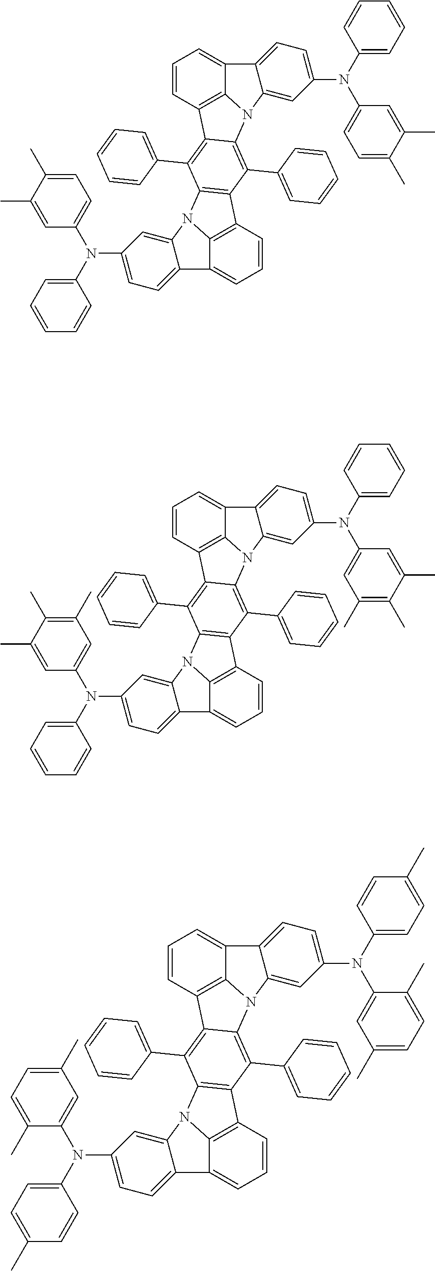

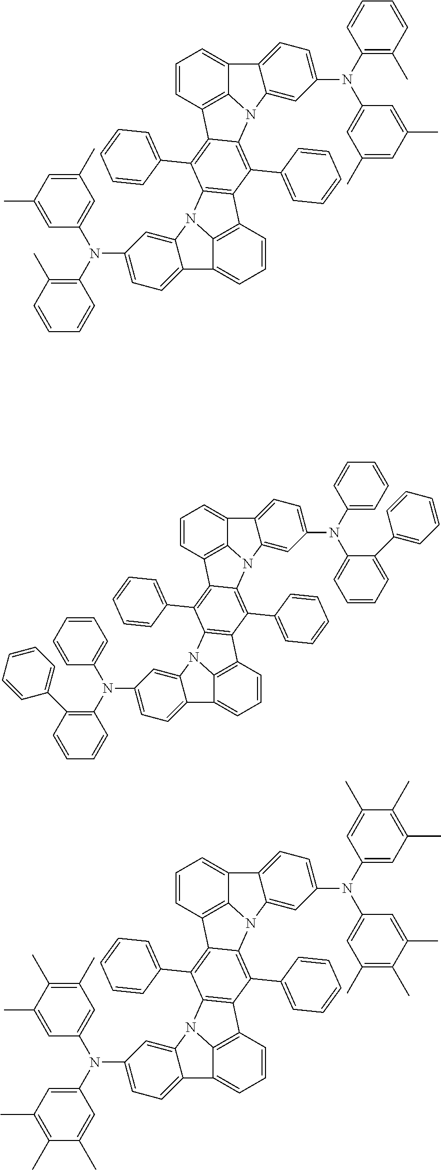

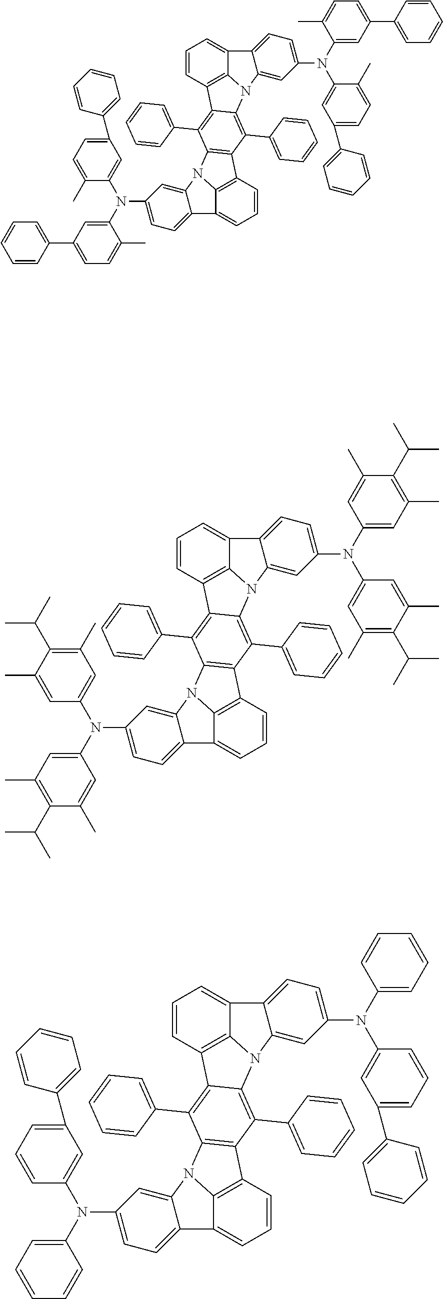

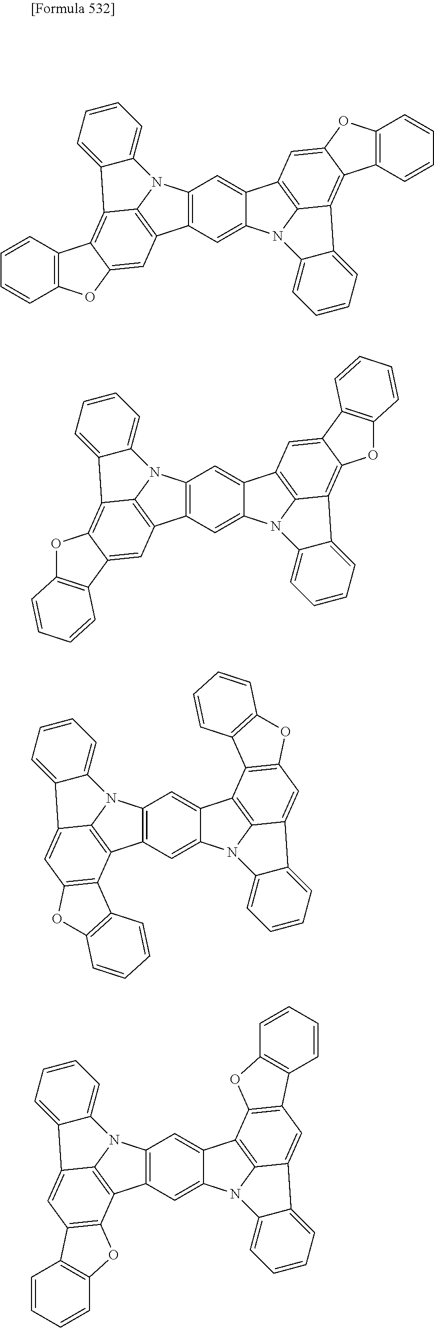

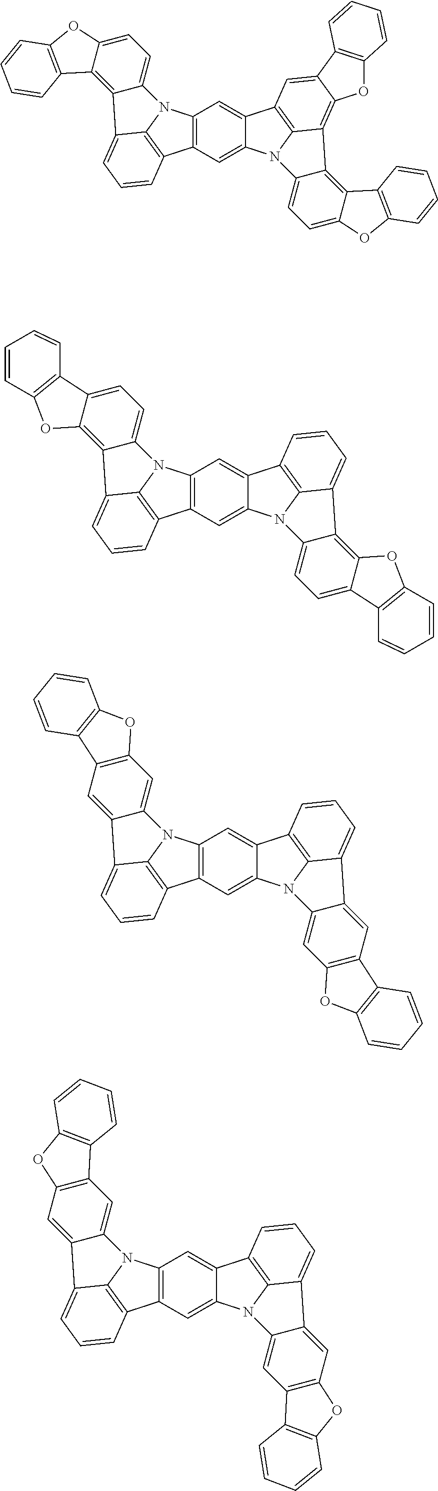

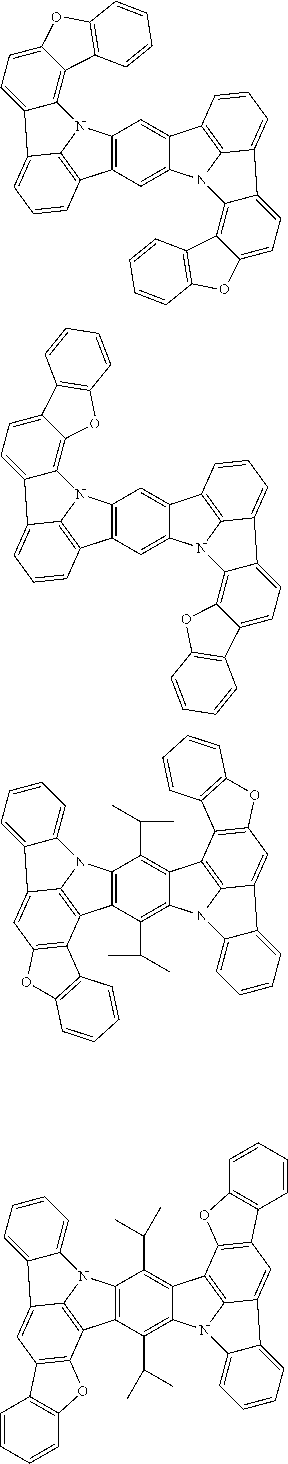

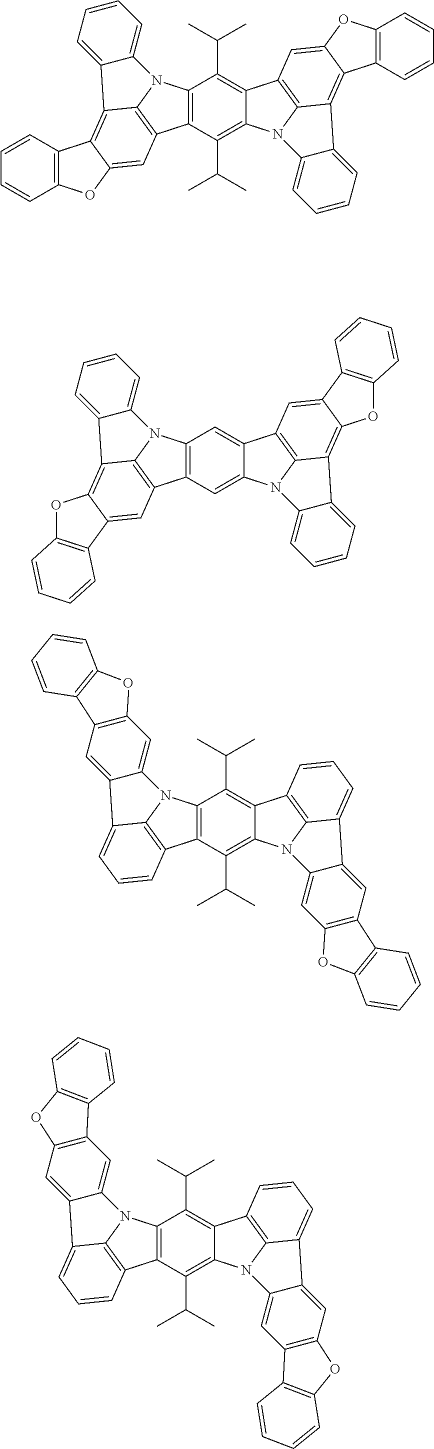

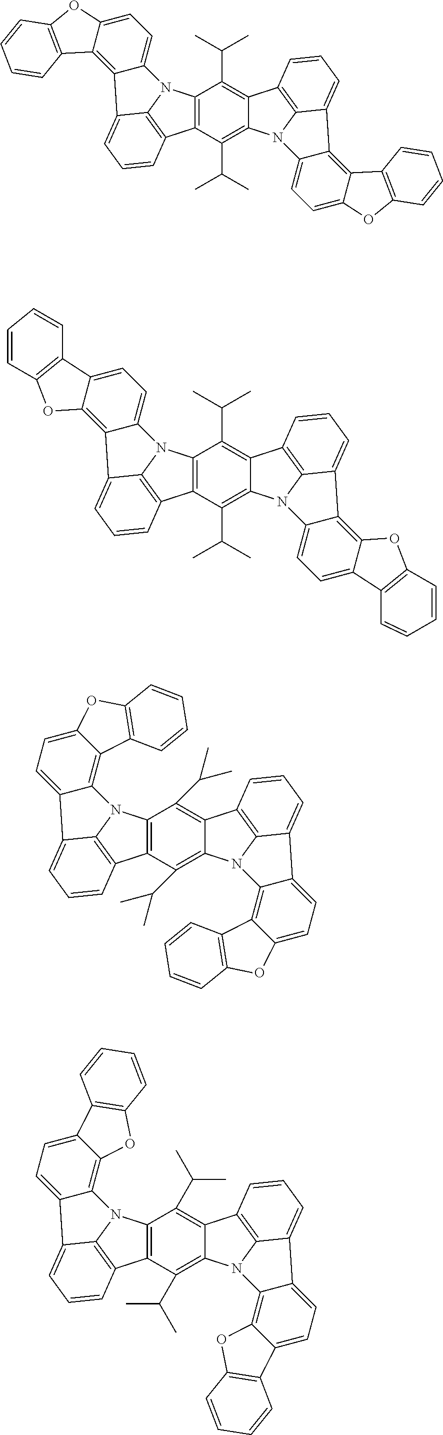

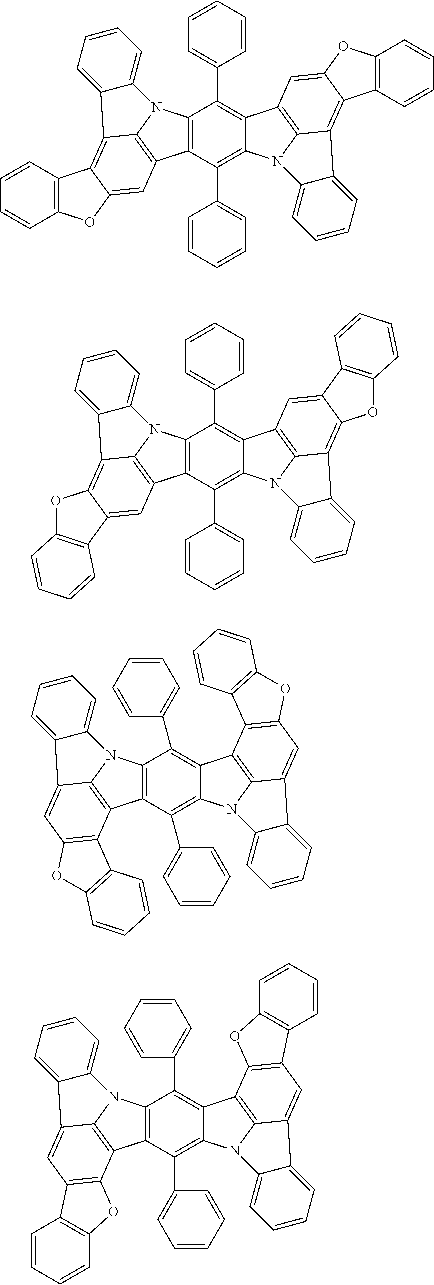

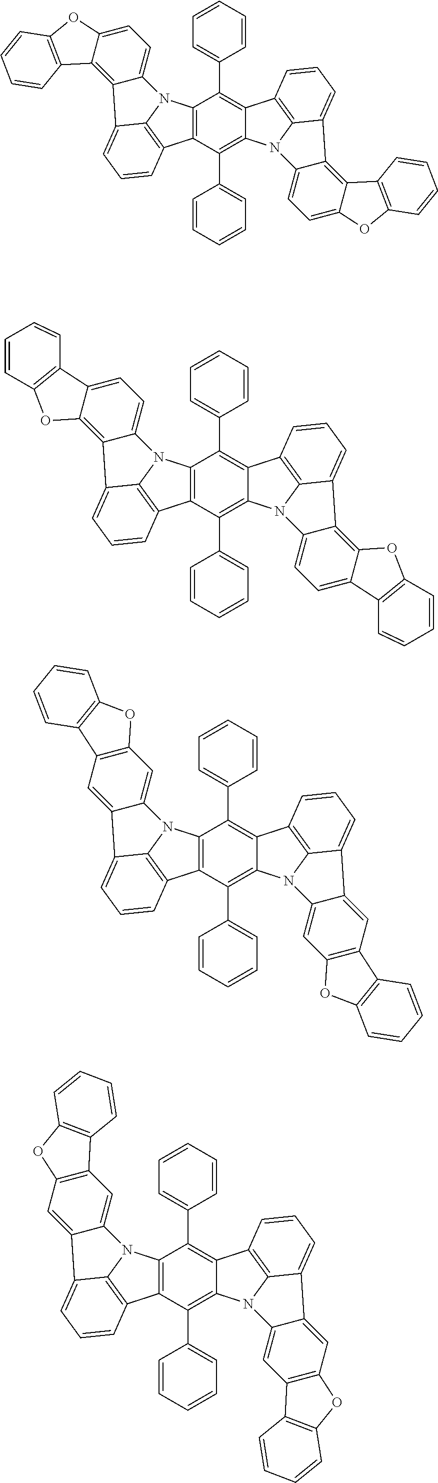

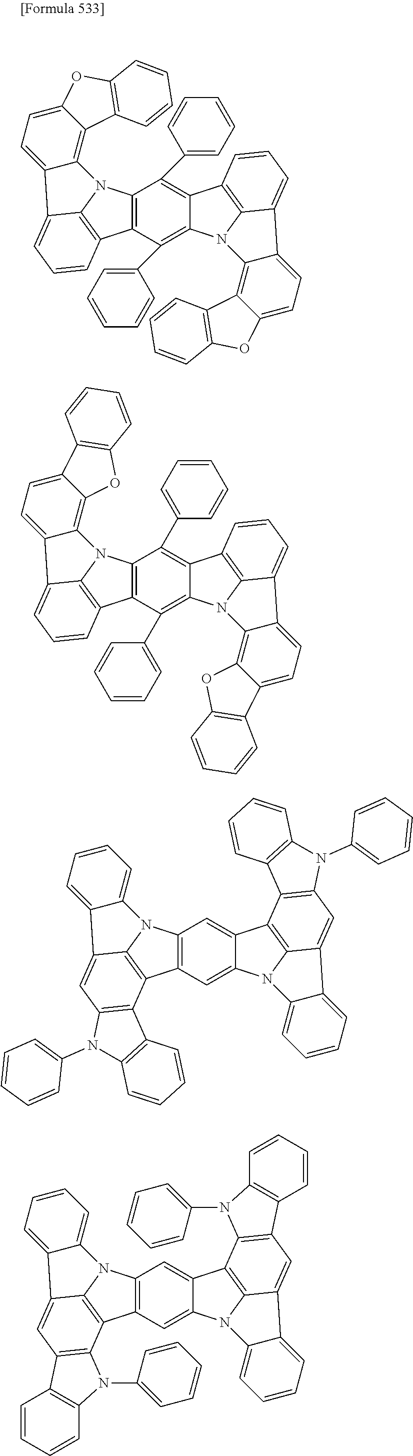

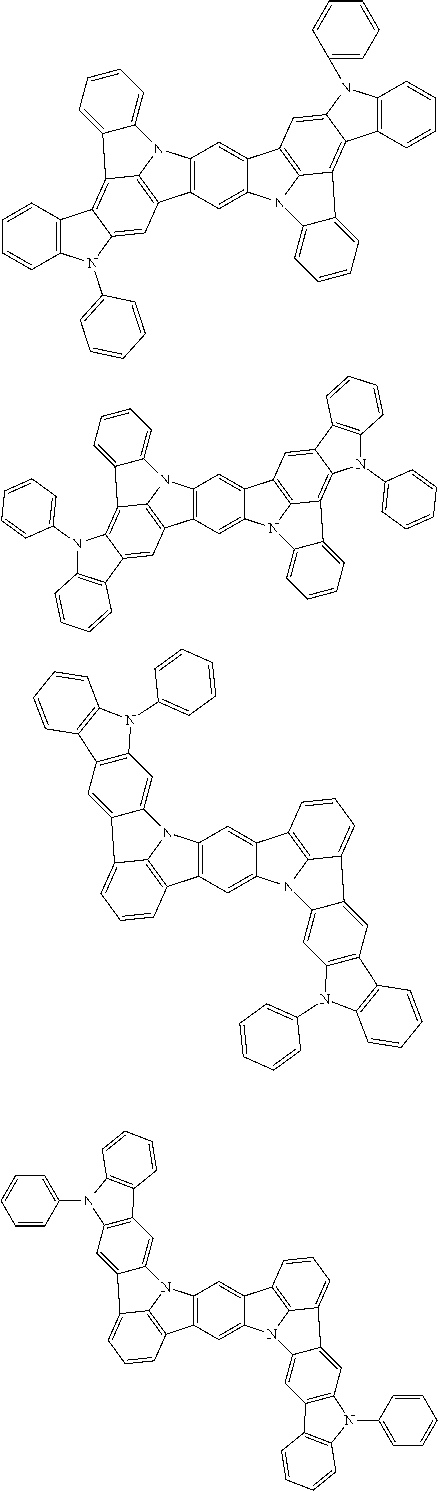

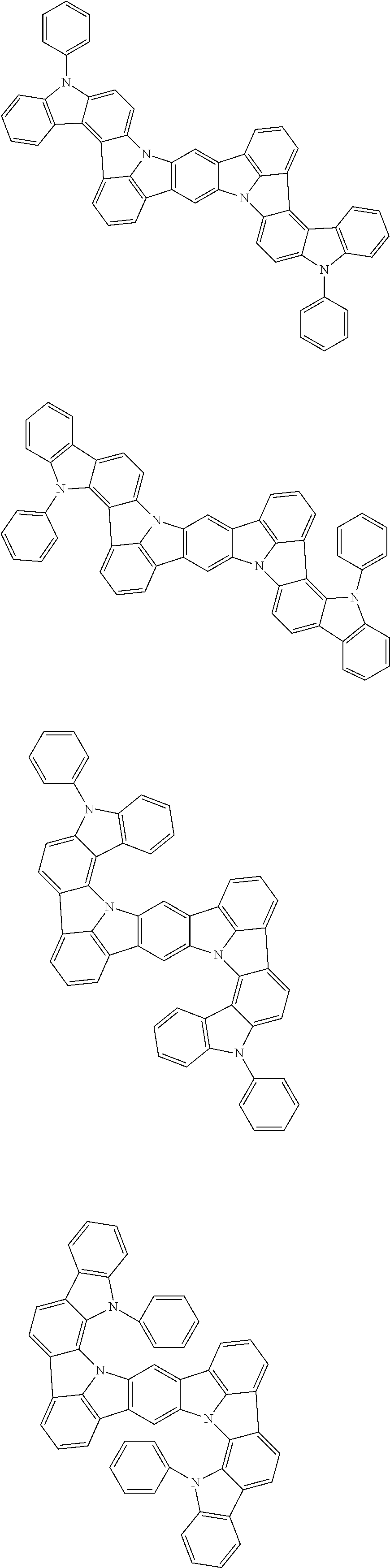

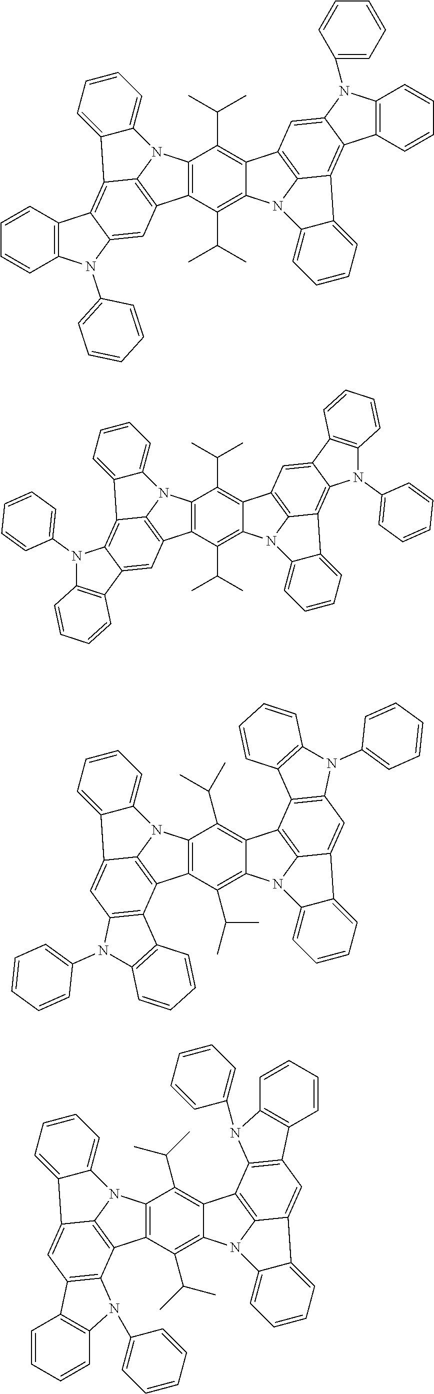

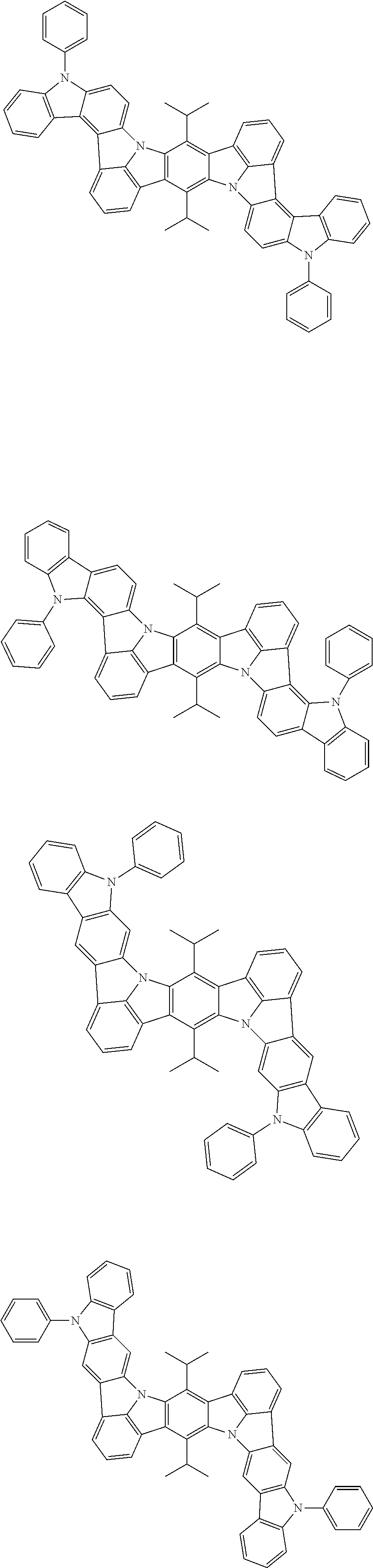

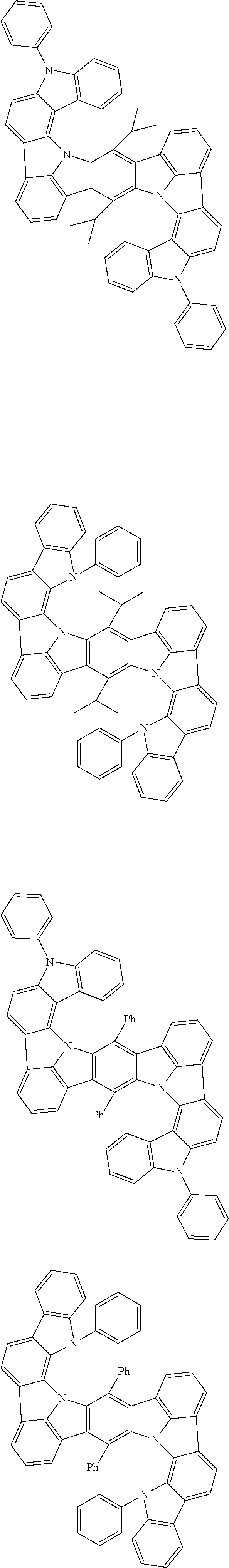

In the formulae, Ph is a phenyl group.

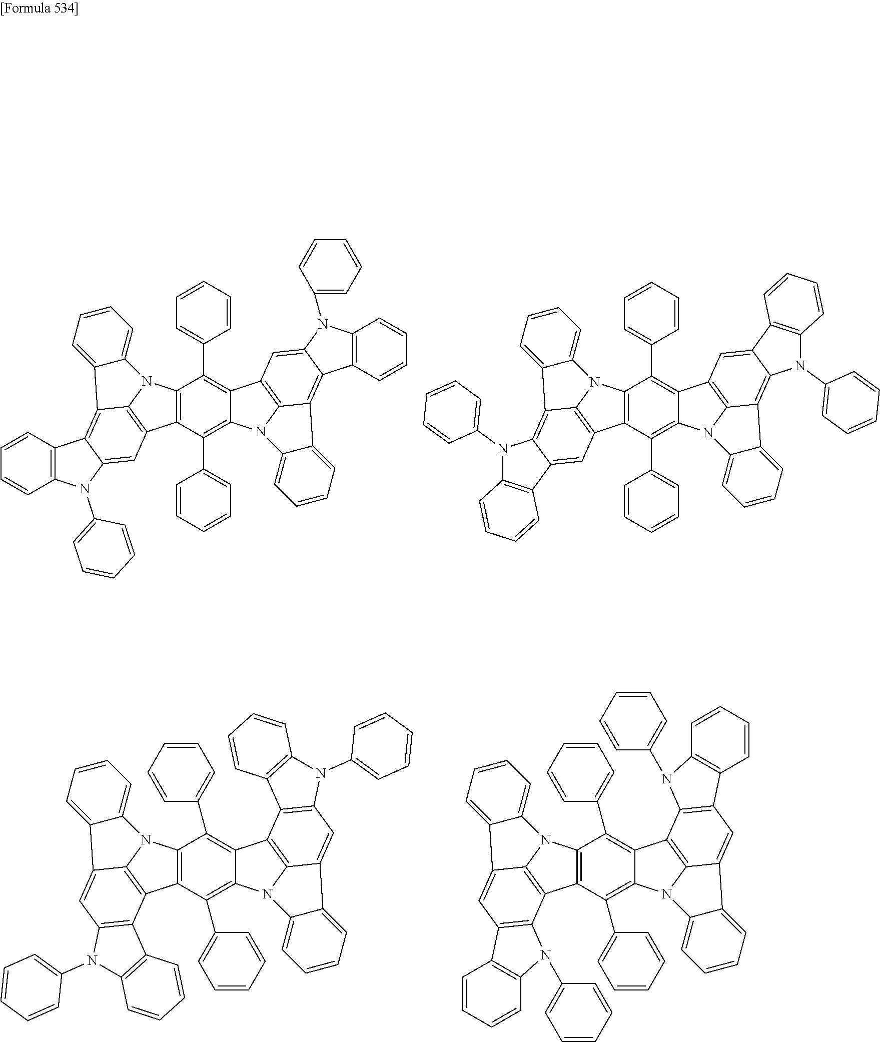

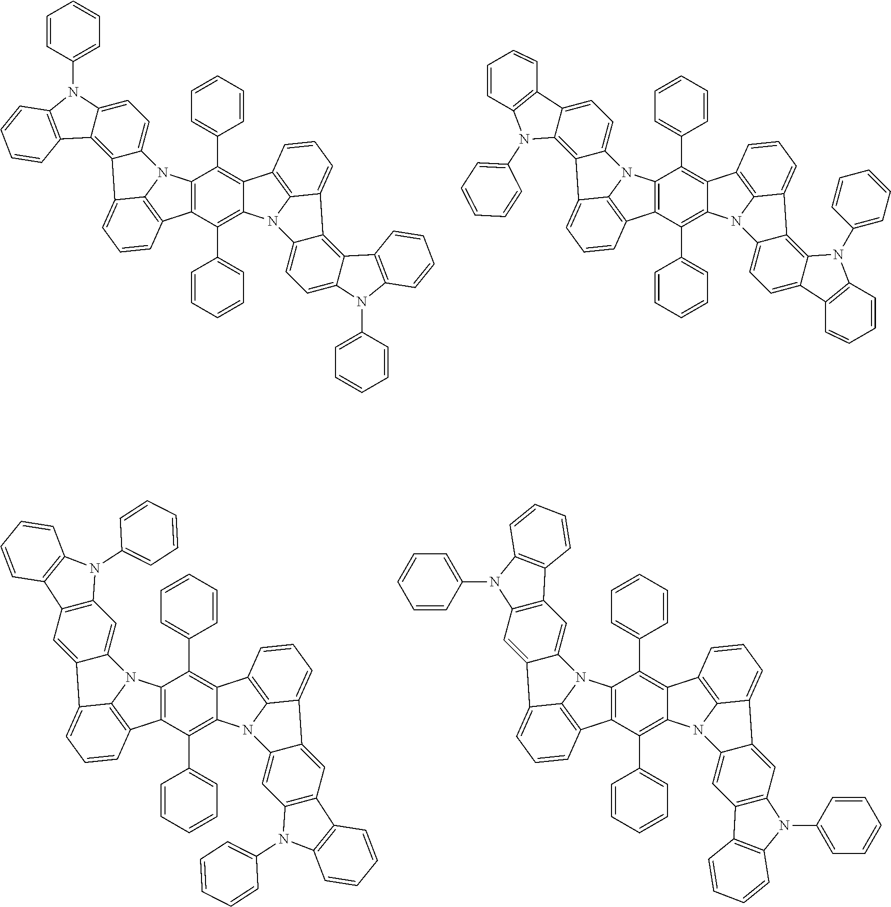

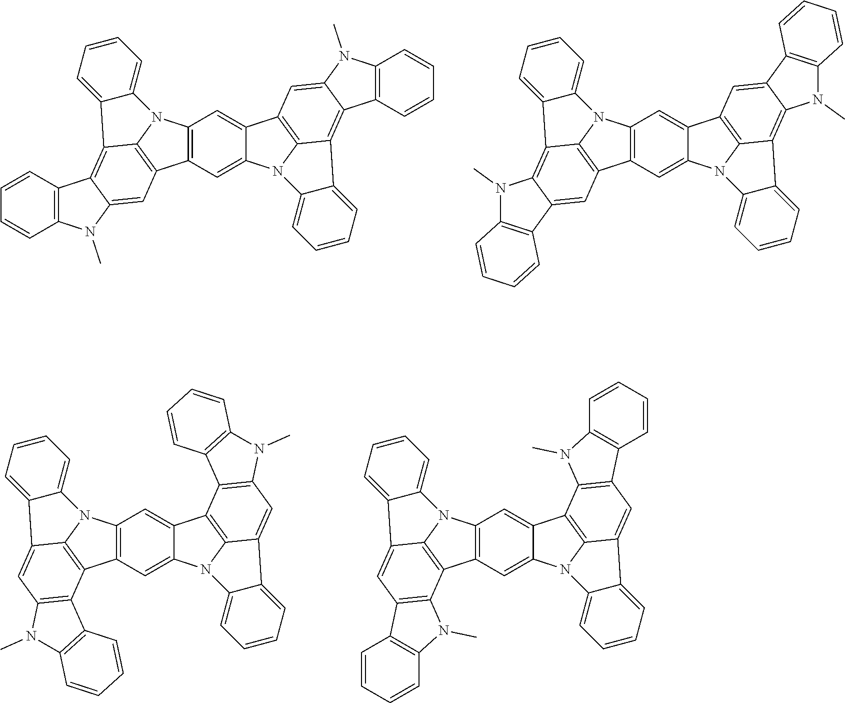

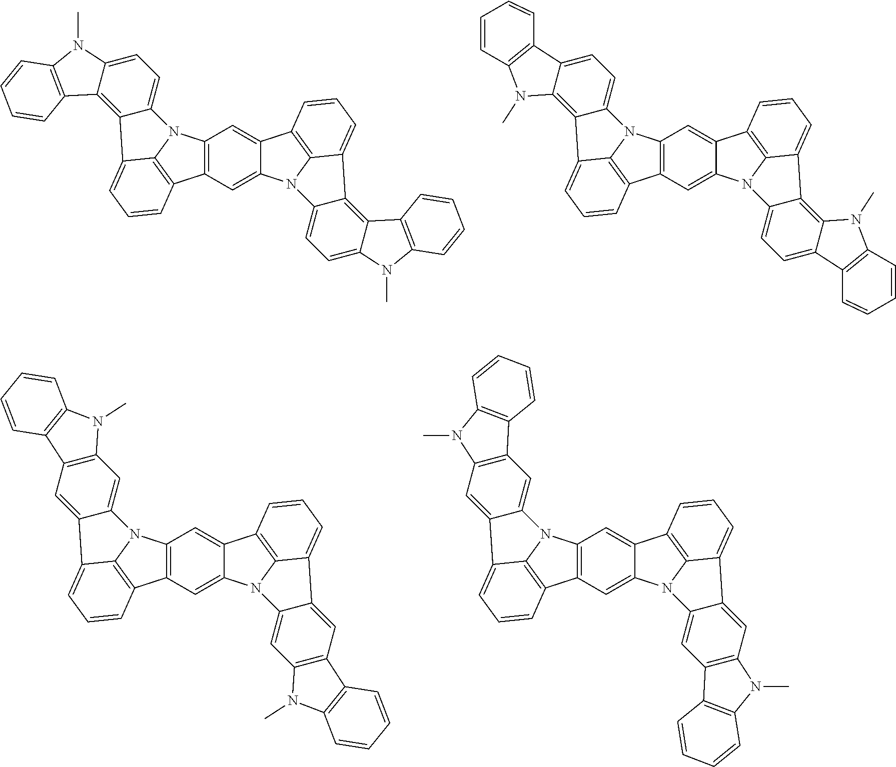

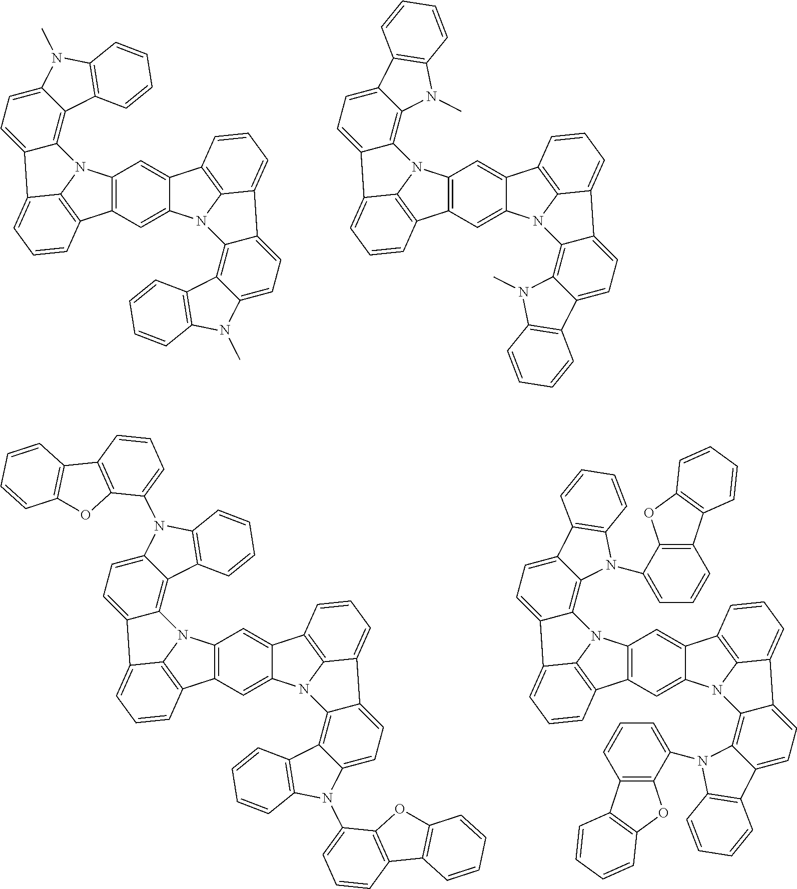 [SMILES: Cn1c2ccccc2c2c1ccc1c3cccc4c5cc6c(cc5n(c43)c12)c1cccc2c3ccc4c(c5ccccc5n4C)c3n6c12 ; Cn1c2ccccc2c2ccc3c4cccc5c6cc7c(cc6n(c54)c3c21)c1cccc2c3ccc4c5ccccc5n(C)c4c3n7c12 ; c1ccc2c(c1)oc1c(-n3c4ccccc4c4c3ccc3c5cccc6c7cc8c(cc7n(c65)c34)c3cccc4c5ccc6c(c7ccccc7n6-c6cccc7c6oc6ccccc67)c5n8c34)cccc12 ; c1ccc2c(c1)oc1c(-n3c4ccccc4c4ccc5c6cccc7c8cc9c(cc8n(c76)c5c43)c3cccc4c5ccc6c7ccccc7n(-c7cccc8c7oc7ccccc78)c6c5n9c34)cccc12]

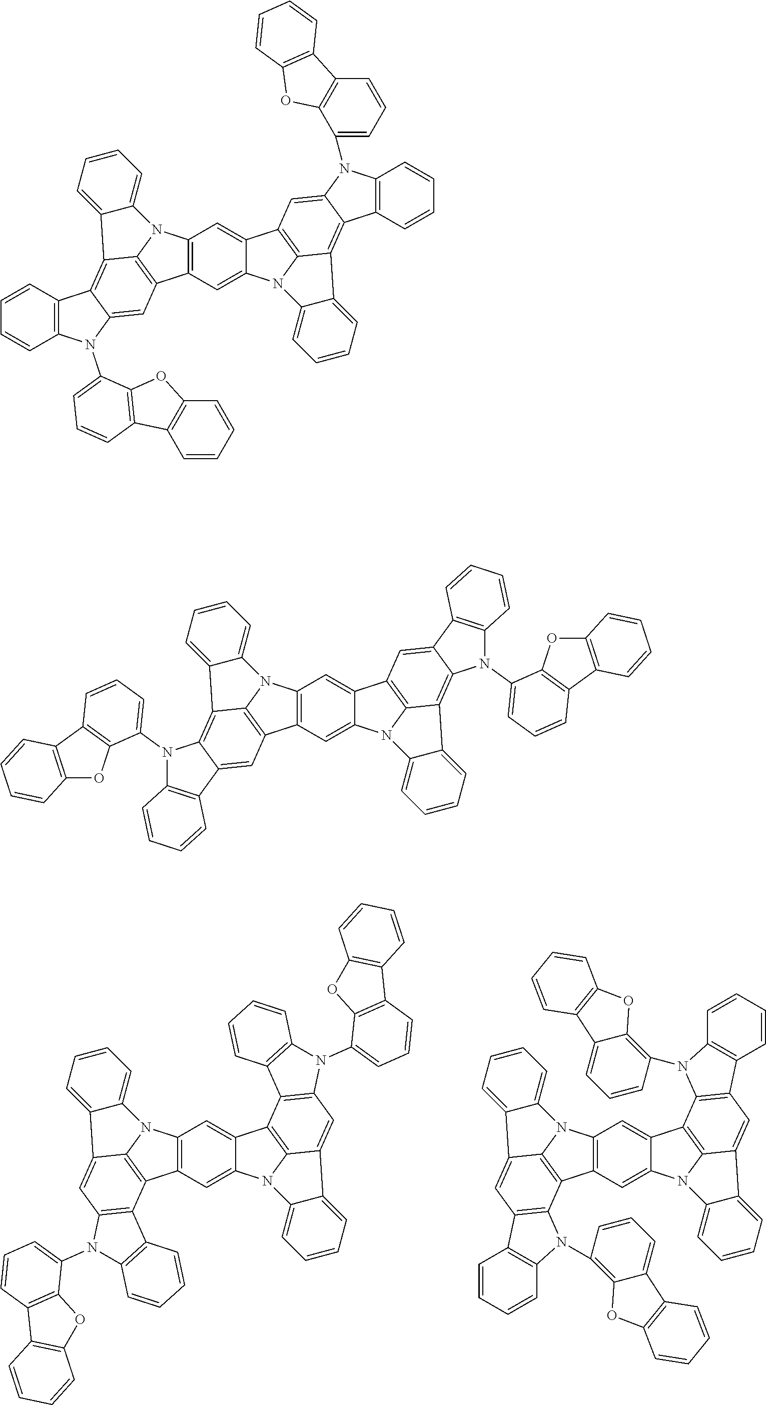

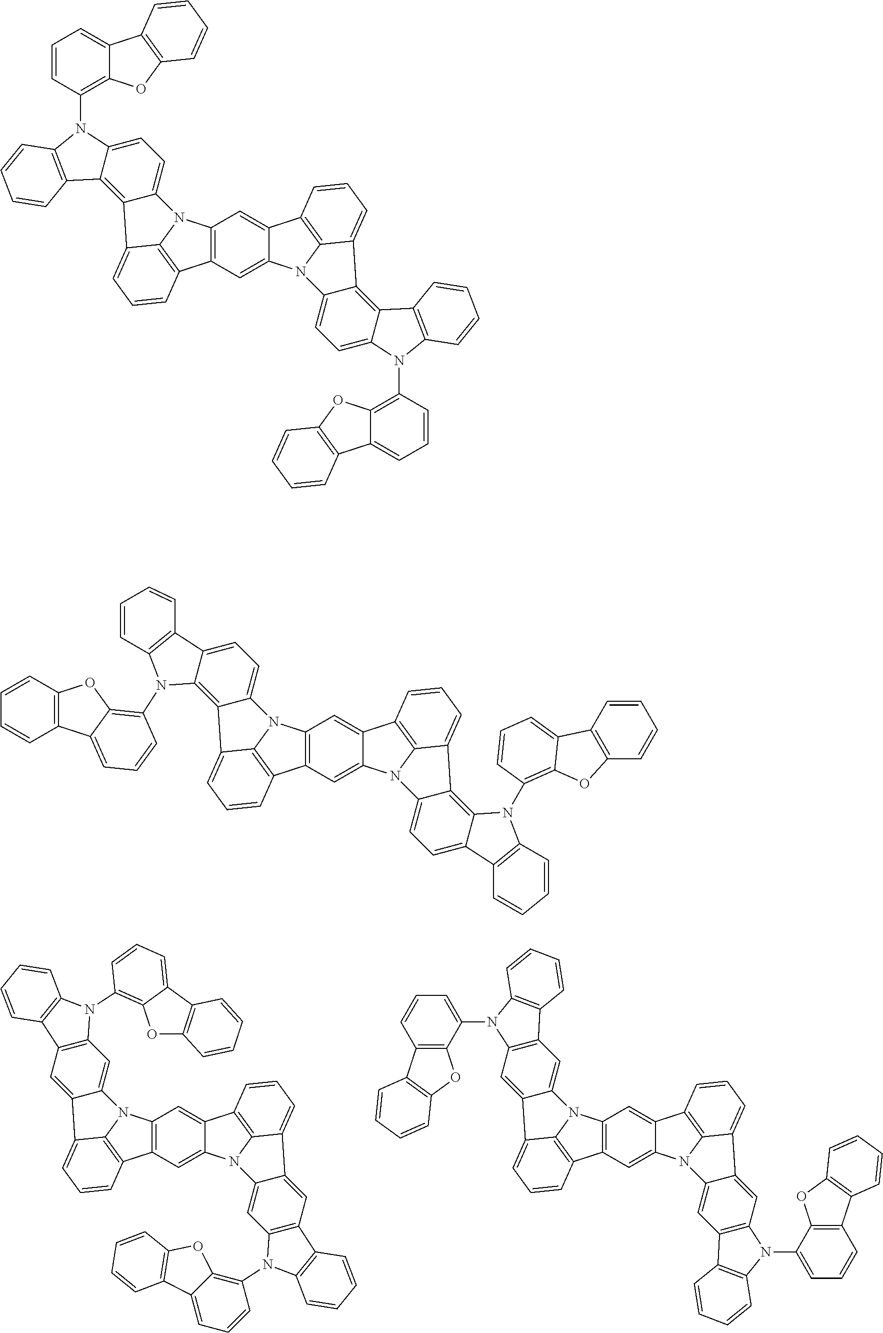

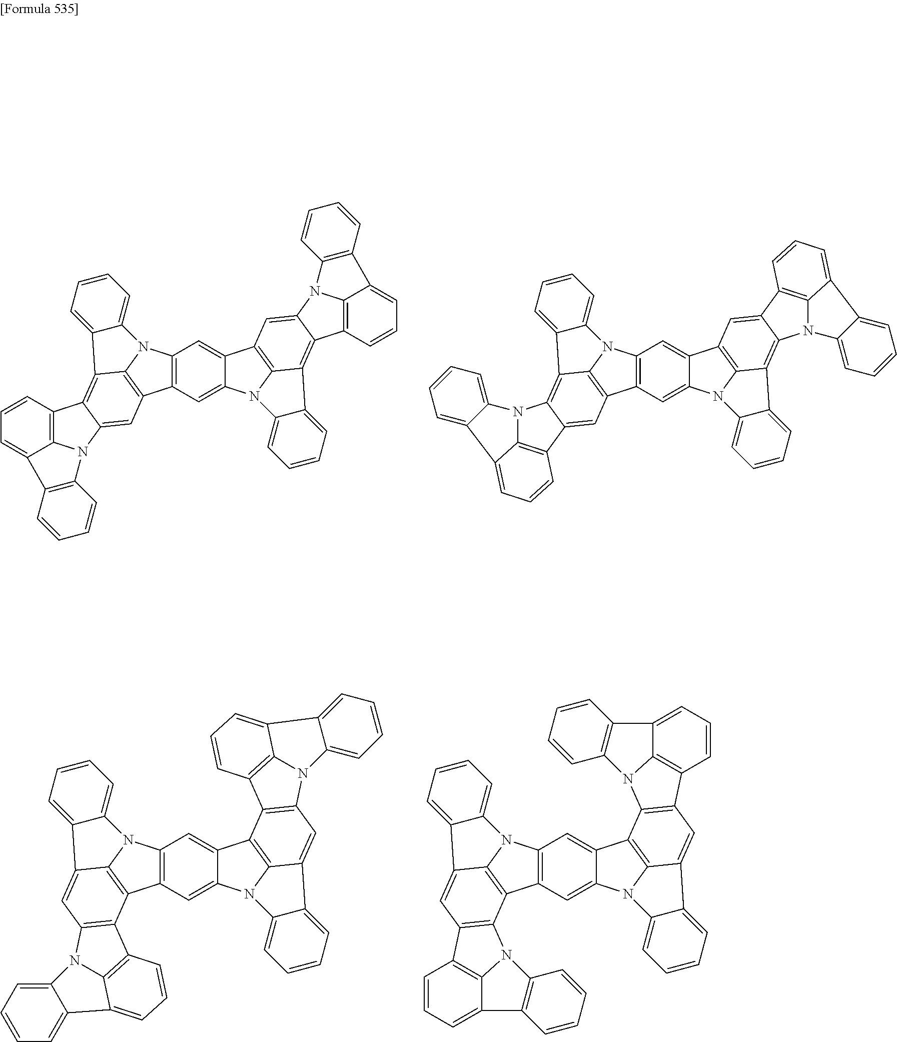 [SMILES: c1ccc2c(c1)c1cccc3c4c5c6ccccc6n6c7cc8c9cc%10c(c%11cccc%12c%13ccccc%13n%10c%12%11)c%10c%11ccccc%11n(c8cc7c(cc4n2c13)c56)c9%10 ; c1ccc2c(c1)c1c3c(cc4c5cccc6c7ccccc7n(c65)c41)c1cc4c(cc1n23)c1cc2c3cccc5c6ccccc6n(c53)c2c2c3ccccc3n4c12 ; c1ccc2c(c1)c1cccc3c4c5c6cc7c(cc6n6c8ccccc8c(cc4n2c13)c56)c1c2c3cccc4c5ccccc5n(c2cc2c5ccccc5n7c21)c43 ; c1ccc2c(c1)c1cc3c4cccc5c6ccccc6n(c54)c3c3c4cc5c(cc4n2c13)c1c2c(cc3c4cccc6c7ccccc7n(c64)c31)c1ccccc1n52]

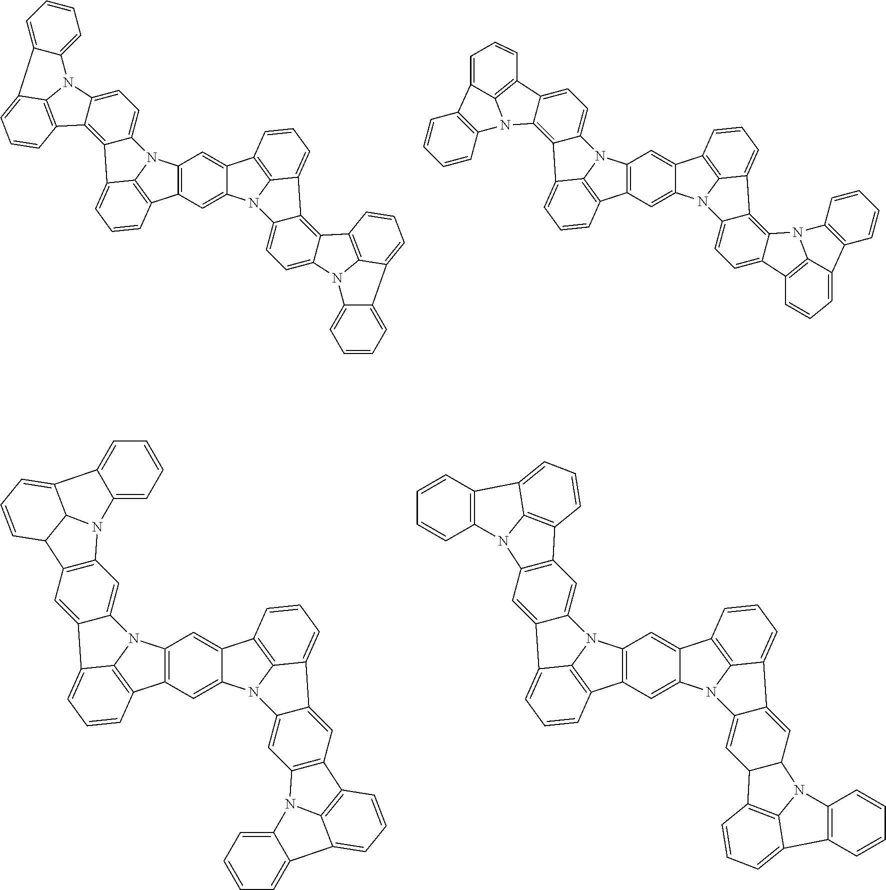 [SMILES: c1ccc2c(c1)c1cccc3c4c5c6cccc7c8cc9c(cc8n(c5ccc4n2c13)c76)c1cccc2c3c4c5cccc6c7ccccc7n(c4ccc3n9c12)c65 ; c1ccc2c(c1)c1cccc3c4ccc5c(c6cccc7c8cc9c(cc8n5c76)c5cccc6c7c(ccc8c%10cccc%11c%12ccccc%12n(c%11%10)c87)n9c56)c4n2c13 ; C1=CC2c3cc4c5cccc6c7cc8c(cc7n(c4cc3N3c4ccccc4C(=C1)C23)c56)c1cccc2c3cc4c5cccc6c7ccccc7n(c4cc3n8c12)c65 ; C1=c2c(n3c4cc5c6cccc7c8cc9c(cc8n(c5cc4c4cccc2c43)c67)c2cccc3c4ccccc4n9c32)=CC2c3cccc4c5ccccc5n(c34)C12]

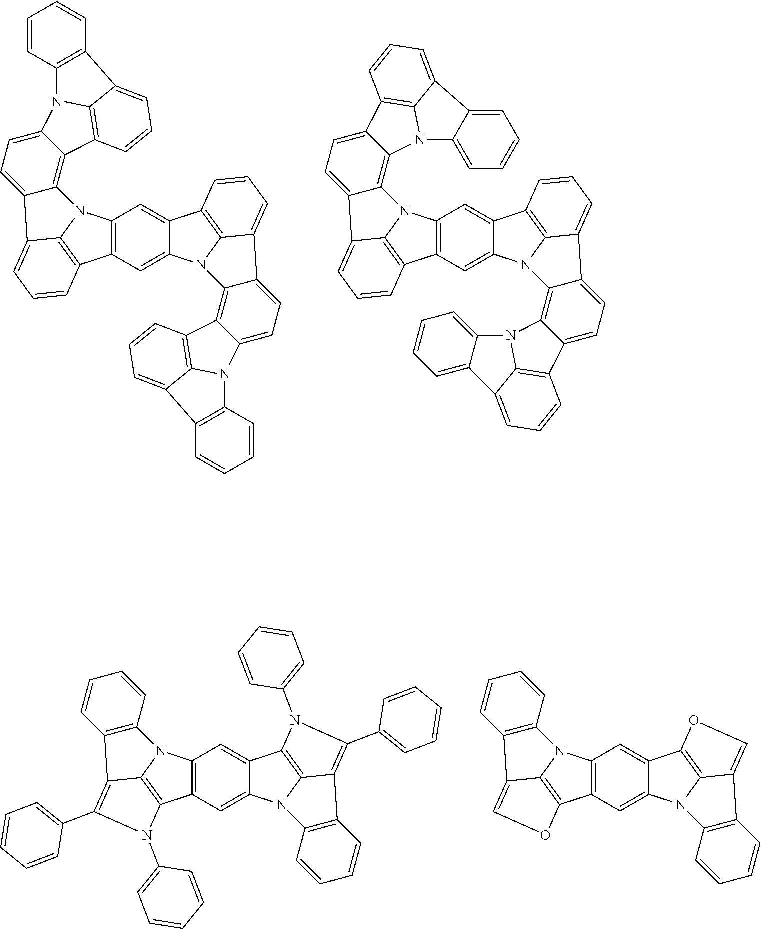

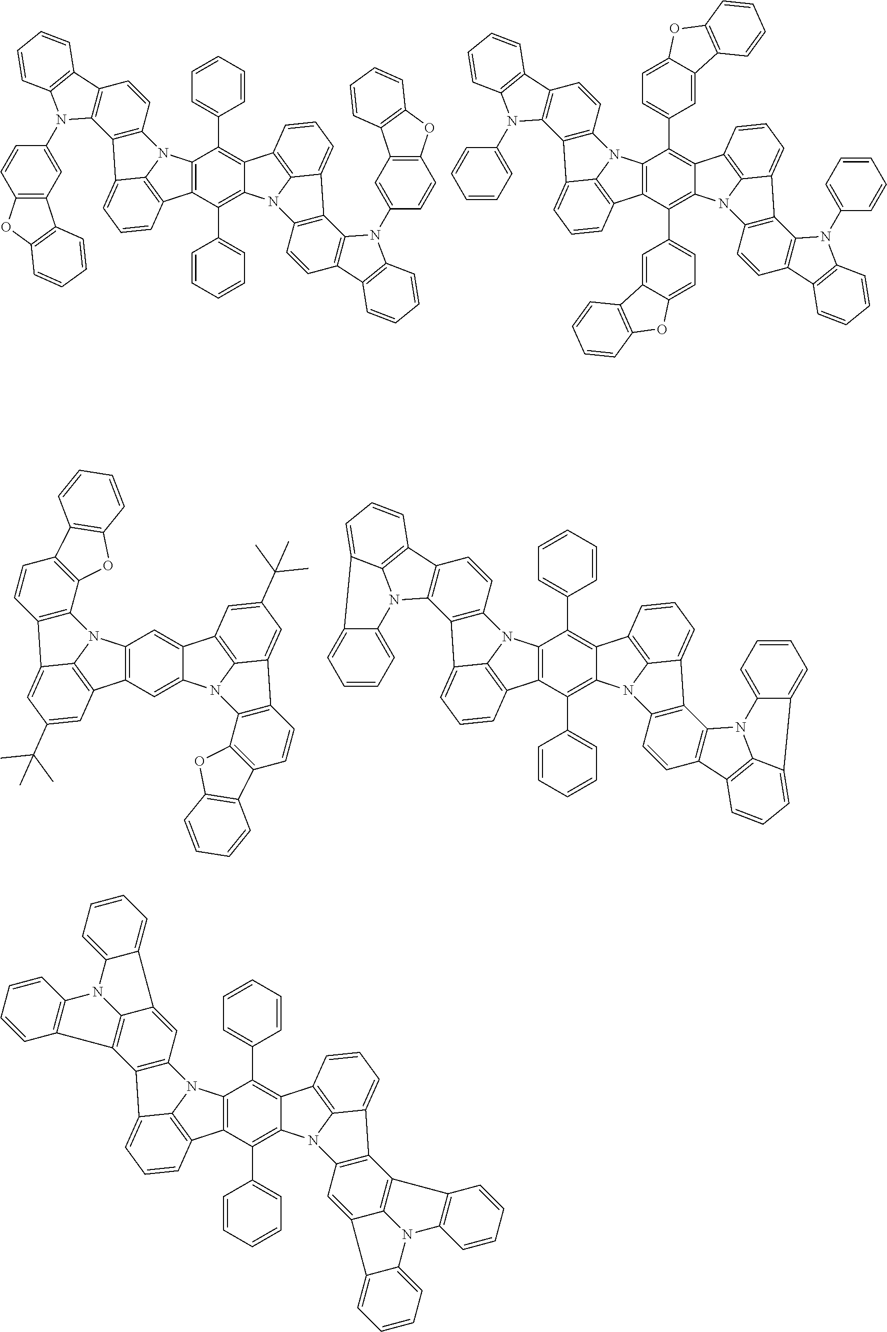

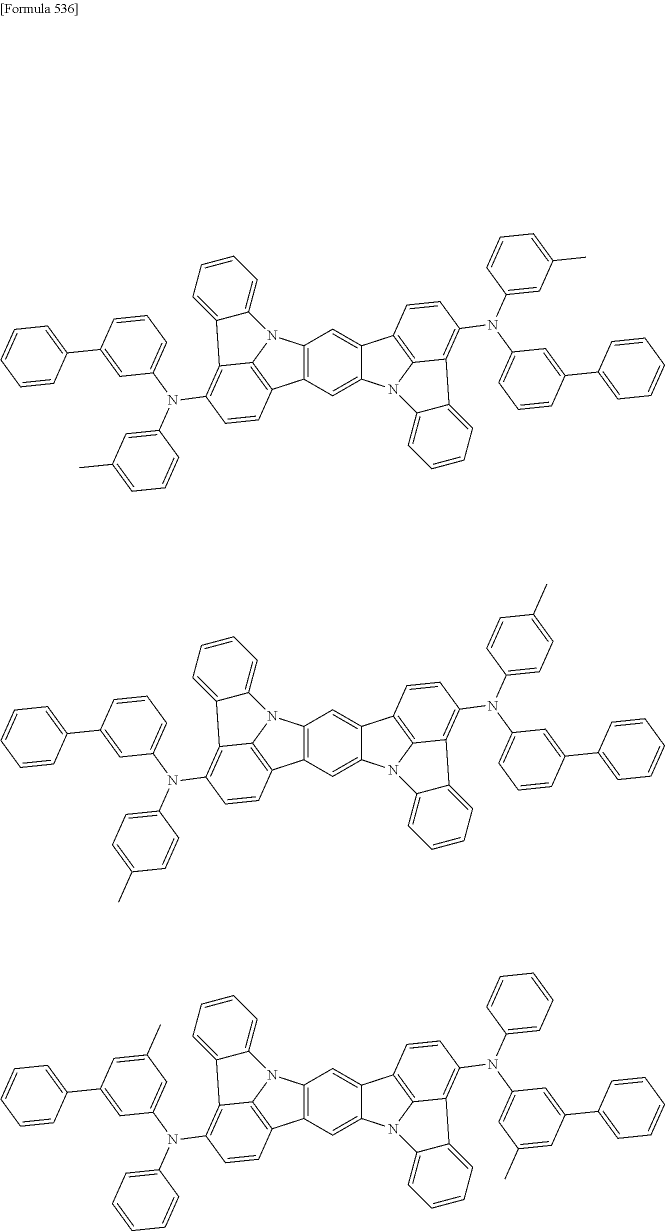 [SMILES: Cc1cccc(N(c2cccc(-c3ccccc3)c2)c2ccc3c4cc5c(cc4n4c6ccccc6c2c34)c2ccc(N(c3cccc(C)c3)c3cccc(-c4ccccc4)c3)c3c4ccccc4n5c23)c1 ; Cc1ccc(N(c2cccc(-c3ccccc3)c2)c2ccc3c4cc5c(cc4n4c6ccccc6c2c34)c2ccc(N(c3ccc(C)cc3)c3cccc(-c4ccccc4)c3)c3c4ccccc4n5c23)cc1 ; Cc1cc(-c2ccccc2)cc(N(c2ccccc2)c2ccc3c4cc5c(cc4n4c6ccccc6c2c34)c2ccc(N(c3ccccc3)c3cc(C)cc(-c4ccccc4)c3)c3c4ccccc4n5c23)c1]

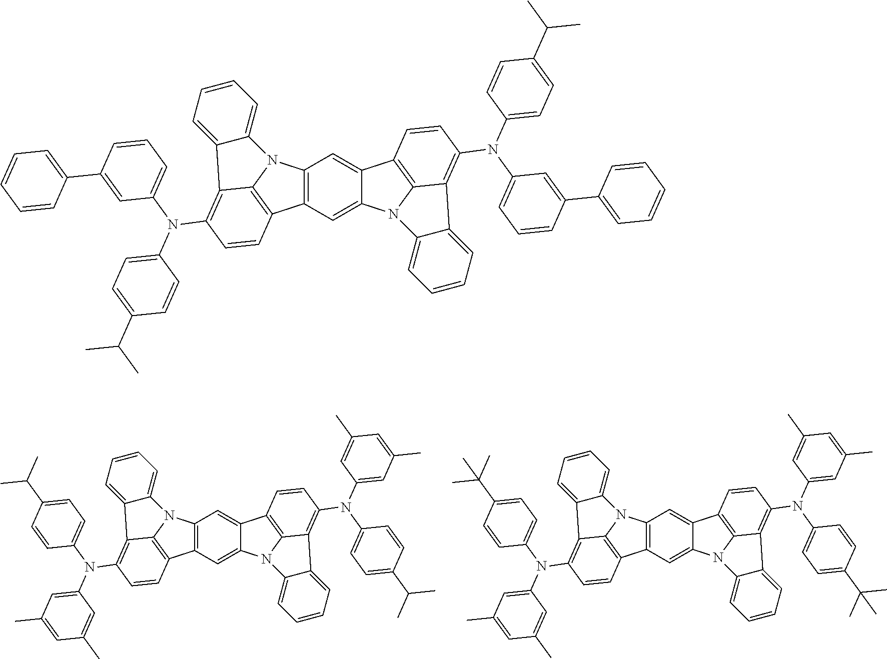 [SMILES: CC(C)c1ccc(N(c2cccc(-c3ccccc3)c2)c2ccc3c4cc5c(cc4n4c6ccccc6c2c34)c2ccc(N(c3ccc(C(C)C)cc3)c3cccc(-c4ccccc4)c3)c3c4ccccc4n5c23)cc1 ; Cc1cc(C)cc(N(c2ccc(C(C)C)cc2)c2ccc3c4cc5c(cc4n4c6ccccc6c2c34)c2ccc(N(c3ccc(C(C)C)cc3)c3cc(C)cc(C)c3)c3c4ccccc4n5c23)c1 ; Cc1cc(C)cc(N(c2ccc(C(C)(C)C)cc2)c2ccc3c4cc5c(cc4n4c6ccccc6c2c34)c2ccc(N(c3ccc(C(C)(C)C)cc3)c3cc(C)cc(C)c3)c3c4ccccc4n5c23)c1]

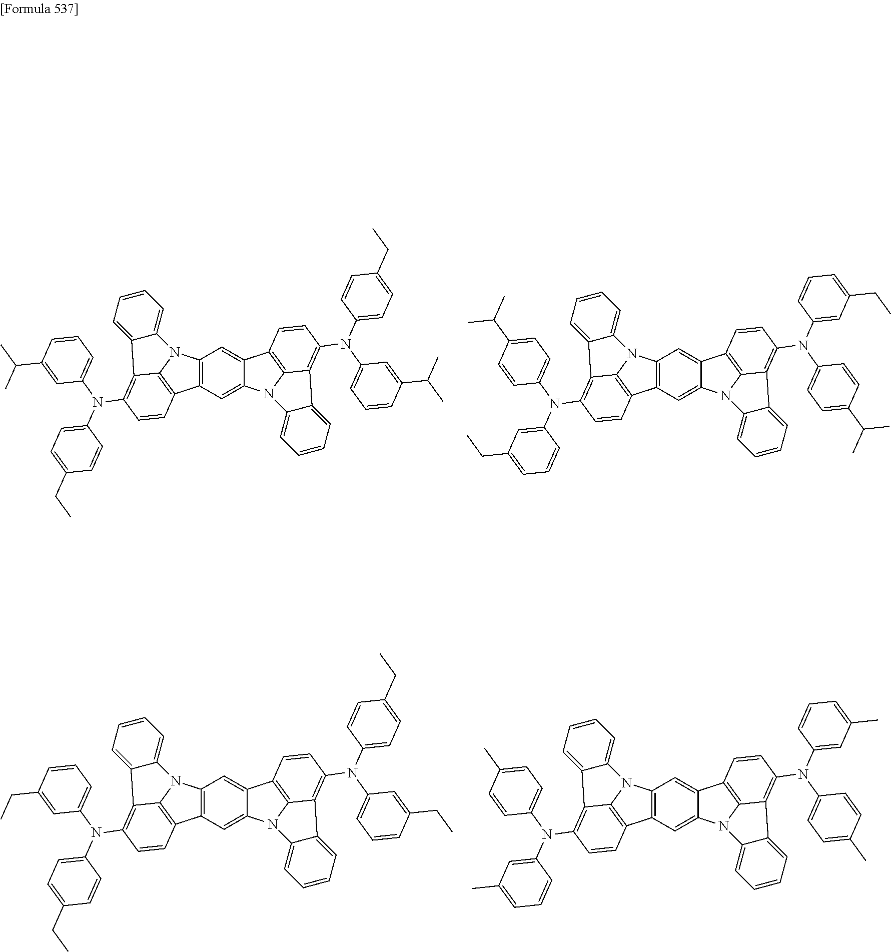

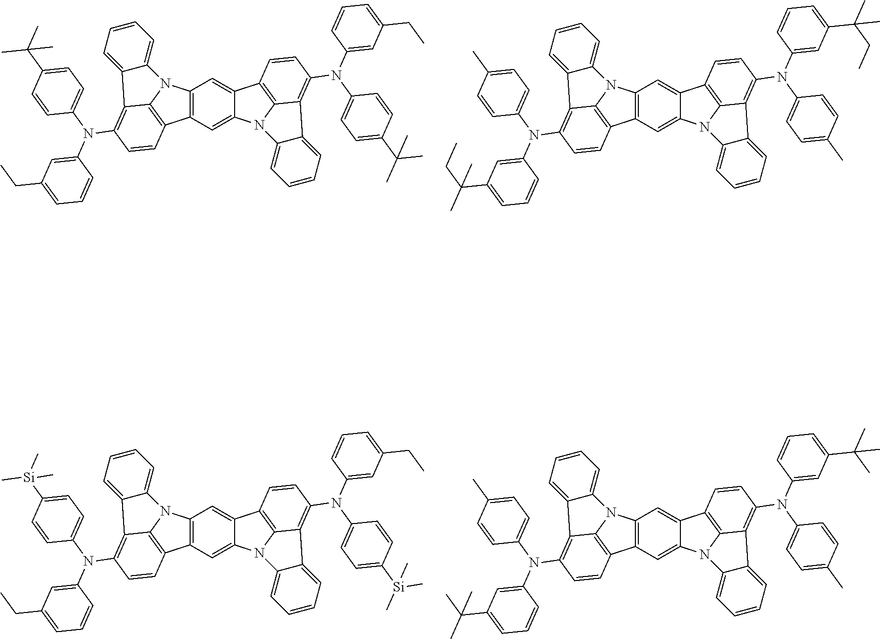

### Compound Represented by Formula (6)

The compound represented by the formula (6) will be described.

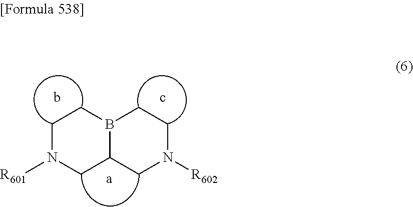

In the formula (6):
a ring, b ring and c ring are each independently a substituted or unsubstituted aromatic hydrocarbon ring having 6 to 50 ring carbon atoms, or a substituted or unsubstituted heterocycle having 5 to 50 ring atoms;
R₆₀₁ and R₆₀₂ are each independently bonded to the a ring, b ring or c ring to form a substituted or unsubstituted heterocycle, or not bonded thereto to form no substituted or unsubstituted heterocycle; and
R₆₀₁ and R₆₀₂ not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

The a ring, b ring and c ring are each a ring (a substituted or unsubstituted aromatic hydrocarbon ring having 6 to 50 ring carbon atoms, or a substituted or unsubstituted heterocycle having 5 to 50 ring atoms) fused with a fused bicyclic structure formed of a boron atom and two nitrogen atoms at the center of the formula (6).

The “aromatic hydrocarbon ring” for the a, b, and c rings has the same structure as a compound formed by introducing a hydrogen atom to the “aryl group” described above.
Ring atoms of the “aromatic hydrocarbon ring” for the a ring include three carbon atoms on the fused bicyclic structure at the center of the formula (6).
Ring atoms of the “aromatic hydrocarbon ring” for the b ring and the c ring include two carbon atoms on the fused bicyclic structure at the center of the formula (6).

Specific examples of the “substituted or unsubstituted aromatic hydrocarbon ring having 6 to 50 ring carbon atoms” include a compound formed by introducing a hydrogen atom to the “aryl group” described in the specific example group G1.

The “heterocycle” for the a, b, and c rings has the same structure as a compound formed by introducing a hydrogen atom to the “heterocyclic group” described above.

Ring atoms of the “heterocycle” for the a ring include three carbon atoms on the fused bicyclic structure at the center of the formula (6). Ring atoms of the “heterocycle” for the b ring and c ring include two carbon atoms on the fused bicyclic structure at the center of the formula (6). Specific examples of the “substituted or unsubstituted heterocycle having 5 to 50 ring atoms” include a compound formed by introducing a hydrogen atom to the “heterocyclic group” described in the specific example group G2.

R₆₀₁ and R₆₀₂ are optionally each independently bonded with the a ring, b ring, or c ring to form a substituted or unsubstituted heterocycle. The “heterocycle” in this arrangement includes a nitrogen atom on the fused bicyclic structure at the center of the formula (6). The heterocycle in the above arrangement optionally includes a hetero atom other than the nitrogen atom. R₆₀₁ and R₆₀₂ bonded with the a ring, b ring, or c ring specifically means that atoms forming R₆₀₁ and R₆₀₂ are bonded with atoms forming the a ring, b ring, or c ring. For instance, R₆₀₁ may be bonded with the a ring to form a bicyclic (or tri-or-more cyclic) fused nitrogen-containing heterocycle, in which the ring including R₆₀₁ and the a ring are fused. Specific examples of the nitrogen-containing heterocycle include a compound corresponding to the nitrogen-containing bi(or-more)cyclic fused heterocyclic group in the specific example group G2.

The same applies to R₆₀₁ bonded with the b ring, R₆₀₂ bonded with the a ring, and R₆₀₂ bonded with the c ring.

In an exemplary embodiment, the a ring, b ring and c ring in the formula (6) are each independently a substituted or unsubstituted aromatic hydrocarbon ring having 6 to 50 ring carbon atoms.

In an exemplary embodiment, the a ring, b ring and c ring in the formula (6) are each independently a substituted or unsubstituted benzene ring or a substituted or unsubstituted naphthalene ring.

In an exemplary embodiment, R₆₀₁ and R₆₀₂ in the formula (6) are each independently a substituted or unsubstituted aryl group having 6 to 50 ring carbon atoms, or a substituted or unsubstituted heterocyclic group having 5 to 50 ring atoms, preferably a substituted or unsubstituted aryl group having 6 to 50 ring carbon atoms.

In an exemplary embodiment, the compound represented by the formula (6) is a compound represented by a formula (62) below.

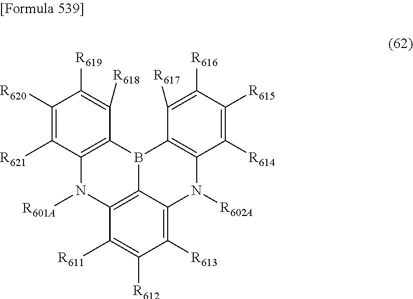

In the formula (62):
R_{601A} is bonded with at least one of R₆₁₁ or R₆₂₁ to form a substituted or unsubstituted heterocycle, or not bonded therewith to form no substituted or unsubstituted heterocycle;
R_{602A} is bonded with at least one of R₆₁₃ or R₆₁₄ to form a substituted or unsubstituted heterocycle, or not bonded therewith to form no substituted or unsubstituted heterocycle;
R_{601A} and R_{602A} not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
at least one combination of adjacent two or more of R₆₁₁ to R₆₂₁ are mutually bonded to form a substituted or unsubstituted monocyclic ring, mutually bonded to form a substituted or unsubstituted fused ring, or not mutually bonded; and
R₆₁₁ to R₆₂₁ not forming the substituted or unsubstituted heterocycle, not forming the monocyclic ring, and not forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

R_{601A} and R_{602A} in the formula (62) are groups corresponding to R₆₀₁ and R₆₀₂ in the formula (6), respectively.

For instance, R_{601A} and R₆₁₁ are optionally bonded with each other to form a bicyclic (or tri-or-more cyclic) fused nitrogen-containing heterocycle, in which the ring including R_{601A} and R₆₁₁ and a benzene ring corresponding to the a ring are fused. Specific examples of the nitrogen-containing heterocycle include a compound corresponding to the nitrogen-containing bi(or-more)cyclic fused heterocyclic group in the specific example group G2. The same applies to R_{601A} bonded with R₆₂₁, R_{602A} bonded with R₆₁₃, and R_{602A} bonded with R₆₁₄.

At least one combination of adjacent two or more of R₆₁₁ to R₆₂₁ may be mutually bonded to form a substituted or unsubstituted monocyclic ring, or mutually bonded to form a substituted or unsubstituted fused ring.

For instance, R₆₁₁ and R₆₁₂ are optionally mutually bonded to form a structure in which a benzene ring, indole ring, pyrrole ring, benzofuran ring, benzothiophene ring or the like is fused to the six-membered ring bonded with R₆₁₁ and R₆₁₂, the resultant fused ring forming a naphthalene ring, carbazole ring, indole ring, dibenzofuran ring, or dibenzothiophene ring, respectively.

In an exemplary embodiment, R₆₁₁ to R₆₂₁ not contributing to ring formation are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary embodiment, R₆₁₁ to R₆₂₁ not contributing to ring formation are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₆₁₁ to R₆₂₁ not contributing to ring formation are each independently a hydrogen atom, or a substituted or unsubstituted alkyl group having 1 to 50 carbon atoms.

In an exemplary embodiment, R₆₁₁ to R₆₂₁ not contributing to ring formation are each independently a hydrogen atom, or a substituted or unsubstituted alkyl group having 1 to 50 carbon atoms, and
at least one of R₆₁₁ to R₆₂₁ is a substituted or unsubstituted alkyl group having 1 to 50 carbon atoms.

In an exemplary embodiment, the compound represented by the formula (62) is a compound represented by a formula (63) below.

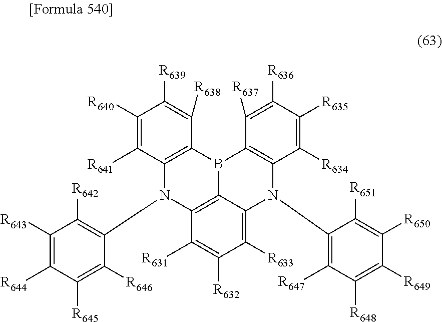

In the formula (63):
R₆₃₁ is bonded with R₆₄₆ to form a substituted or unsubstituted heterocycle, or not bonded therewith to form no substituted or unsubstituted heterocycle;
R₆₃₃ is bonded with R₆₄₇ to form a substituted or unsubstituted heterocycle, or not bonded therewith to form no substituted or unsubstituted heterocycle;
R₆₃₄ is bonded with R₆₅₁ to form a substituted or unsubstituted heterocycle, or not bonded therewith to form no substituted or unsubstituted heterocycle;
R₆₄₁ is bonded with R₆₄₂ to form a substituted or unsubstituted heterocycle, or not bonded therewith to form no substituted or unsubstituted heterocycle;
at least one combination of adjacent two or more of R₆₃₁ to R₆₅₁ are mutually bonded to form a substituted or unsubstituted monocyclic ring, mutually bonded to form a substituted or unsubstituted fused ring, or not mutually bonded; and
R₆₃₁ to R₆₅₁ not forming the substituted or unsubstituted heterocycle, not forming the monocyclic ring, and not forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

R₆₃₁ are optionally bonded with R₆₄₆ to form a substituted or unsubstituted heterocycle. For instance, R₆₃₁ and R₆₄₆ are optionally bonded with each other to form a tri-or-more cyclic fused nitrogen-containing heterocycle, in which a benzene ring bonded with R₆₄₆, a ring including a nitrogen atom, and a benzene ring corresponding to the a ring are fused. Specific examples of the nitrogen-containing heterocycle include a compound corresponding to the nitrogen-containing tri(-or-more)cyclic fused heterocyclic group in the specific example group G2. The same applies to R₆₃₃ bonded with R₆₄₇, R₆₃₄ bonded with R₆₅₁, and R₆₄₁ bonded with R₆₄₂.

In an exemplary embodiment, R₆₃₁ to R₆₅₁ not contributing to ring formation are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary embodiment, R₆₃₁ to R₆₅₁ not contributing to ring formation are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₆₃₁ to R₆₅₁ not contributing to ring formation are each independently a hydrogen atom, or a substituted or unsubstituted alkyl group having 1 to 50 carbon atoms.

In an exemplary embodiment, R₆₃₁ to R₆₅₁ not contributing to ring formation are each independently a hydrogen atom, or a substituted or unsubstituted alkyl group having 1 to 50 carbon atoms, and
at least one of R₆₃₁ to R₆₅₁ is a substituted or unsubstituted alkyl group having 1 to 50 carbon atoms.

In an exemplary embodiment, the compound represented by the formula (63) is a compound represented by a formula (63A) below.

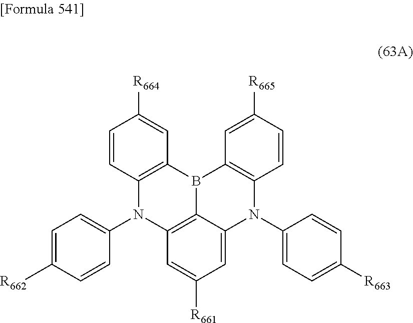

In the formula (63A):
R₆₆₁ is a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms, and
R₆₆₂ to R₆₆₅ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary embodiment, R₆₆₁ to R₆₆₅ are each independently a substituted or unsubstituted alkyl group having 1 to 50 carbon atoms, or a substituted or unsubstituted aryl group having 6 to 50 ring carbon atoms.

In an exemplary embodiment, R₆₆₁ to R₆₆₅ are each independently a substituted or unsubstituted alkyl group having 1 to 50 carbon atoms.

In an exemplary embodiment, the compound represented by the formula (63) is a compound represented by a formula (63B) below.

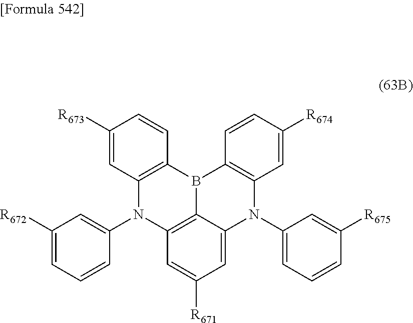

In the formula (63B):
R₆₇₁ and R₆₇₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms; and
R₆₇₃ to R₆₇₅ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms.

In an exemplary embodiment, the compound represented by the formula (63) is a compound represented by a formula (63B′) below.

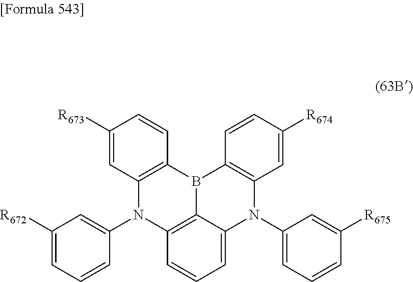

In the formula (63B′), R₆₇₂ to R₆₇₅ each independently represent the same as R₆₇₂ to R₆₇₅ in the formula (63B).

In an exemplary embodiment, at least one of R₆₇₁ to R₆₇₅ is a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms.

In an exemplary embodiment, R₆₇₂ is a hydrogen atom, a substituted or unsubstituted alkyl group having 1 to 50 carbon atoms, a group represented by —N(R₉₀₆)(R₉₀₇), or a substituted or unsubstituted aryl group having 6 to 50 ring carbon atoms; and
R₆₇₁ and R₆₇₃ to R₆₇₅ are each independently a substituted or unsubstituted alkyl group having 1 to 50 carbon atoms, a group represented by —N(R₉₀₆)(R₉₀₇), or a substituted or unsubstituted aryl group having 6 to 50 ring carbon atoms.

In an exemplary embodiment, the compound represented by the formula (63) is a compound represented by a formula (63C) below.

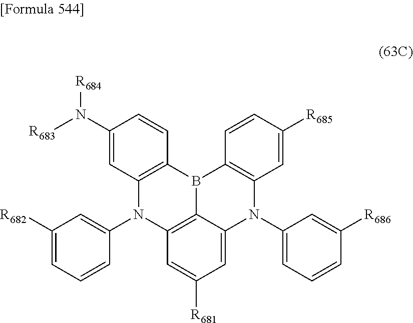

In the formula (63C):
R₆₈₁ and R₆₈₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms; and
R₆₈₃ to R₆₈₆ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary embodiment, the compound represented by the formula (63) is a compound represented by a formula (63C′) below.

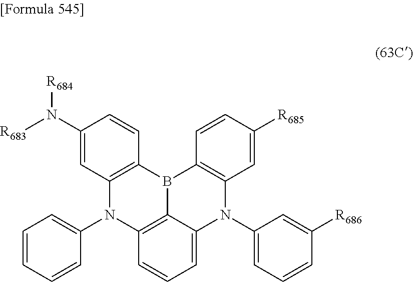

In the formula (63C′), R₆₈₃ to R₆₈₆ each independently represent the same as R₆₈₃ to R₆₈₆ in the formula (63C).

In an exemplary embodiment, R₆₈₁ to R₆₈₆ are each independently a substituted or unsubstituted alkyl group having 1 to 50 carbon atoms, or a substituted or unsubstituted aryl group having 6 to 50 ring carbon atoms.

In an exemplary embodiment, R₆₈₁ to R₆₈₆ are each independently a substituted or unsubstituted aryl group having 6 to 50 ring carbon atoms.

The compound represented by the formula (6) is producible by initially bonding the a ring, b ring and c ring with linking groups (a group including N—R₆₀₁ and a group including N—R₆₀₂) to form an intermediate (first reaction), and bonding the a ring, b ring and c ring with a linking group (a group including a boron atom) to form a final product (second reaction). In the first reaction, an amination reaction (e.g. Buchwald-Hartwig reaction) is applicable. In the second reaction, Tandem Hetero-Friedel-Crafts Reactions or the like is applicable.

Specific examples of the compound represented by the formula (6) are shown below. It should however be noted that these specific examples are merely exemplary and do not limit the compound represented by the formula (6).

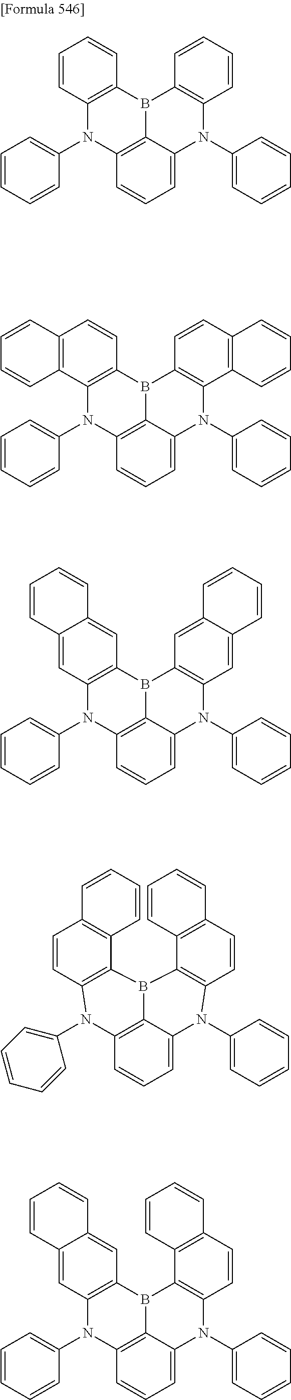 [SMILES: c1ccc(N2c3ccccc3B3c4ccccc4N(c4ccccc4)c4cccc2c43)cc1 ; c1ccc(N2c3cccc4c3B(c3ccc5ccccc5c32)c2ccc3ccccc3c2N4c2ccccc2)cc1 ; c1ccc(N2c3cc4ccccc4cc3B3c4cc5ccccc5cc4N(c4ccccc4)c4cccc2c43)cc1 ; c1ccc(N2c3cccc4c3B(c3c2ccc2ccccc32)c2c(ccc3ccccc23)N4c2ccccc2)cc1 ; c1ccc(N2c3cc4ccccc4cc3B3c4c2cccc4N(c2ccccc2)c2ccc4ccccc4c23)cc1]

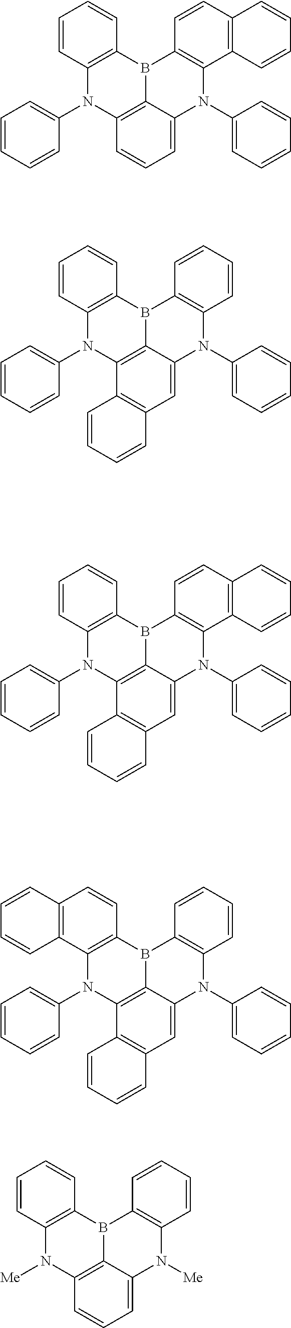 [SMILES: c1ccc(N2c3ccccc3B3c4ccc5ccccc5c4N(c4ccccc4)c4cccc2c43)cc1 ; c1ccc(N2c3ccccc3B3c4ccccc4N(c4ccccc4)c4c3c2cc2ccccc42)cc1 ; c1ccc(N2c3cc4ccccc4c4c3B(c3ccccc3N4c3ccccc3)c3ccc4ccccc4c32)cc1 ; c1ccc(N2c3ccccc3B3c4ccc5ccccc5c4N(c4ccccc4)c4c3c2cc2ccccc42)cc1 ; CN1c2ccccc2B2c3ccccc3N(C)c3cccc1c32]

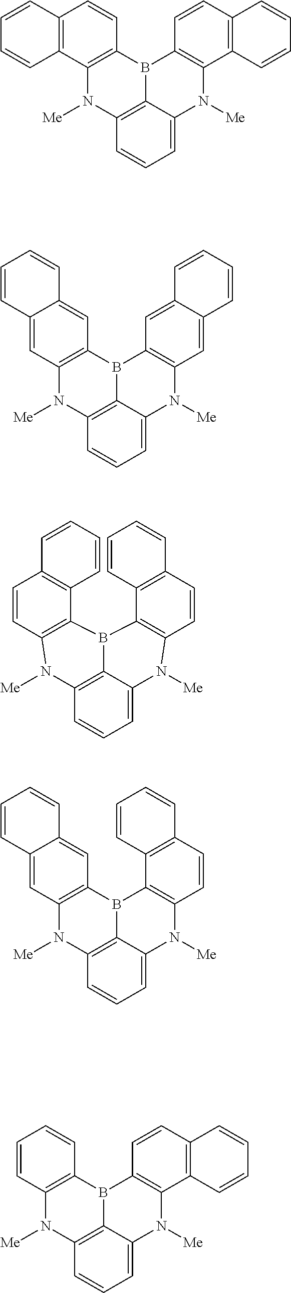 [SMILES: CN1c2cccc3c2B(c2ccc4ccccc4c21)c1ccc2ccccc2c1N3C ; CN1c2cc3ccccc3cc2B2c3cc4ccccc4cc3N(C)c3cccc1c32 ; CN1c2cccc3c2B(c2c1ccc1ccccc21)c1c(ccc2ccccc12)N3C ; CN1c2cc3ccccc3cc2B2c3c1cccc3N(C)c1ccc3ccccc3c12 ; CN1c2ccccc2B2c3ccc4ccccc4c3N(C)c3cccc1c32]

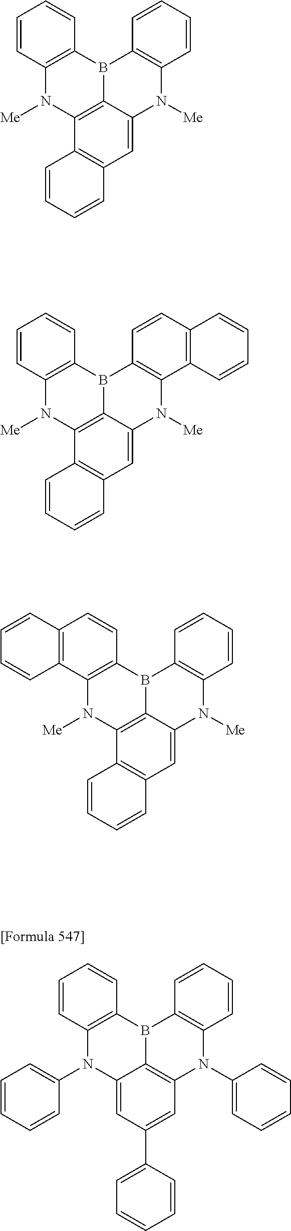 [SMILES: CN1c2ccccc2B2c3ccccc3N(C)c3c2c1cc1ccccc31 ; CN1c2cc3ccccc3c3c2B(c2ccccc2N3C)c2ccc3ccccc3c21 ; CN1c2ccccc2B2c3ccc4ccccc4c3N(C)c3c2c1cc1ccccc31 ; c1ccc(-c2cc3c4c(c2)N(c2ccccc2)c2ccccc2B4c2ccccc2N3c2ccccc2)cc1]

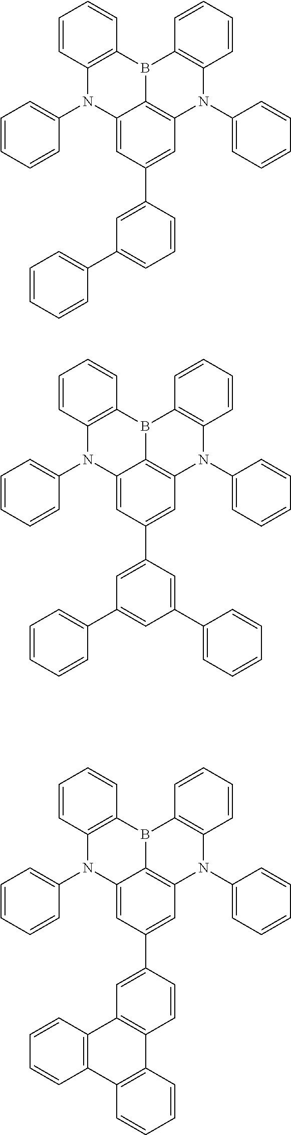 [SMILES: c1ccc(-c2cccc(-c3cc4c5c(c3)N(c3ccccc3)c3ccccc3B5c3ccccc3N4c3ccccc3)c2)cc1 ; c1ccc(-c2cc(-c3ccccc3)cc(-c3cc4c5c(c3)N(c3ccccc3)c3ccccc3B5c3ccccc3N4c3ccccc3)c2)cc1 ; c1ccc(N2c3ccccc3B3c4ccccc4N(c4ccccc4)c4cc(-c5ccc6c7ccccc7c7ccccc7c6c5)cc2c43)cc1]

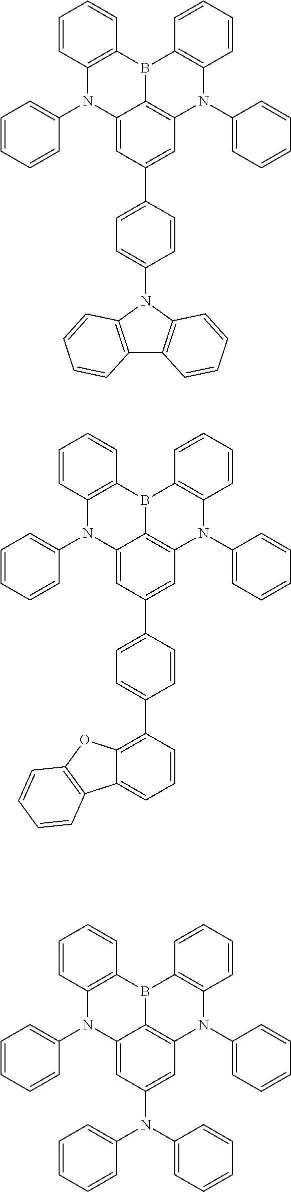 [SMILES: c1ccc(N2c3ccccc3B3c4ccccc4N(c4ccccc4)c4cc(-c5ccc(-n6c7ccccc7c7ccccc76)cc5)cc2c43)cc1 ; c1ccc(N2c3ccccc3B3c4ccccc4N(c4ccccc4)c4cc(-c5ccc(-c6cccc7c6oc6ccccc67)cc5)cc2c43)cc1 ; c1ccc(N(c2ccccc2)c2cc3c4c(c2)N(c2ccccc2)c2ccccc2B4c2ccccc2N3c2ccccc2)cc1]

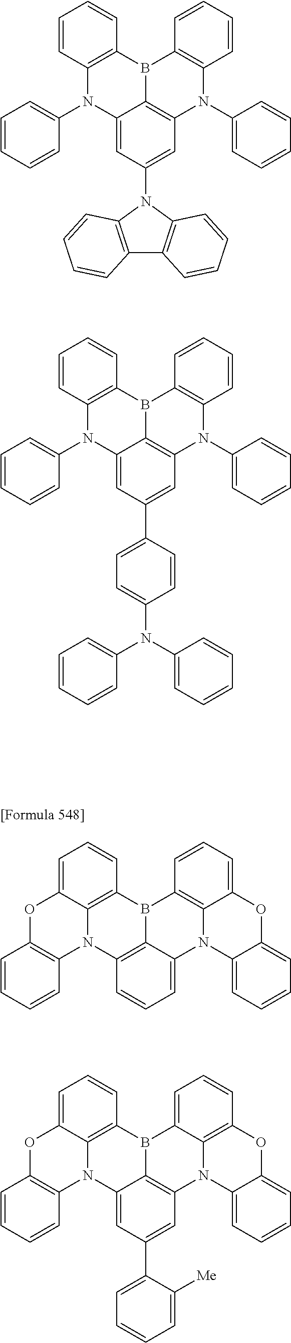 [SMILES: c1ccc(N2c3ccccc3B3c4ccccc4N(c4ccccc4)c4cc(-n5c6ccccc6c6ccccc65)cc2c43)cc1 ; c1ccc(N(c2ccccc2)c2ccc(-c3cc4c5c(c3)N(c3ccccc3)c3ccccc3B5c3ccccc3N4c3ccccc3)cc2)cc1 ; c1ccc2c(c1)Oc1cccc3c1N2c1cccc2c1B3c1cccc3c1N2c1ccccc1O3 ; Cc1ccccc1-c1cc2c3c(c1)N1c4ccccc4Oc4cccc(c41)B3c1cccc3c1N2c1ccccc1O3]

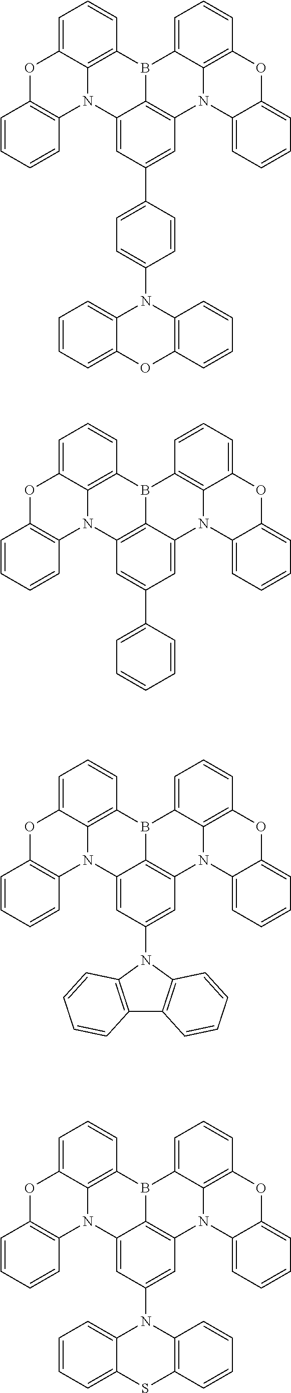 [SMILES: c1ccc2c(c1)Oc1ccccc1N2c1ccc(-c2cc3c4c(c2)N2c5ccccc5Oc5cccc(c52)B4c2cccc4c2N3c2ccccc2O4)cc1 ; c1ccc(-c2cc3c4c(c2)N2c5ccccc5Oc5cccc(c52)B4c2cccc4c2N3c2ccccc2O4)cc1 ; c1ccc2c(c1)Oc1cccc3c1N2c1cc(-n2c4ccccc4c4ccccc42)cc2c1B3c1cccc3c1N2c1ccccc1O3 ; c1ccc2c(c1)Oc1cccc3c1N2c1cc(N2c4ccccc4Sc4ccccc42)cc2c1B3c1cccc3c1N2c1ccccc1O3]

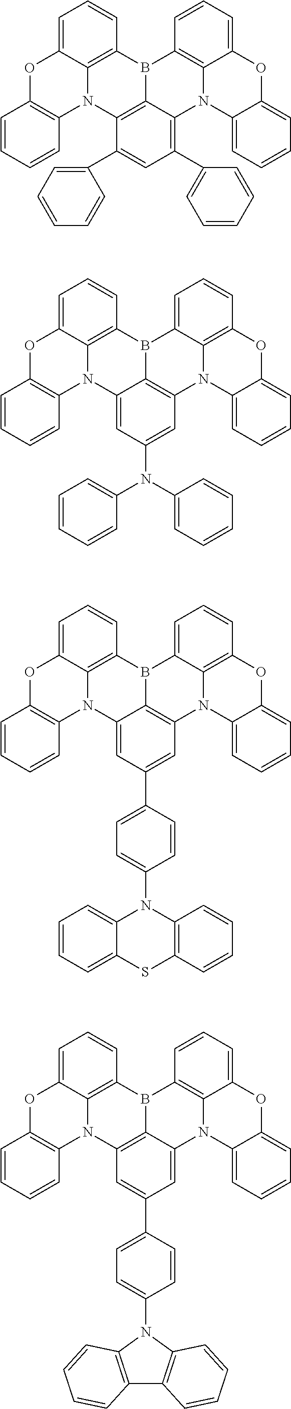 [SMILES: c1ccc(-c2cc(-c3ccccc3)c3c4c2N2c5ccccc5Oc5cccc(c52)B4c2cccc4c2N3c2ccccc2O4)cc1 ; c1ccc(N(c2ccccc2)c2cc3c4c(c2)N2c5ccccc5Oc5cccc(c52)B4c2cccc4c2N3c2ccccc2O4)cc1 ; c1ccc2c(c1)Oc1cccc3c1N2c1cc(-c2ccc(N4c5ccccc5Sc5ccccc54)cc2)cc2c1B3c1cccc3c1N2c1ccccc1O3 ; c1ccc2c(c1)Oc1cccc3c1N2c1cc(-c2ccc(-n4c5ccccc5c5ccccc54)cc2)cc2c1B3c1cccc3c1N2c1ccccc1O3]

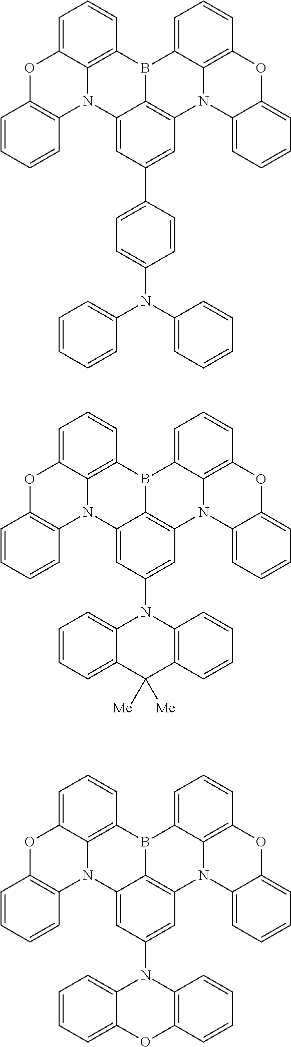

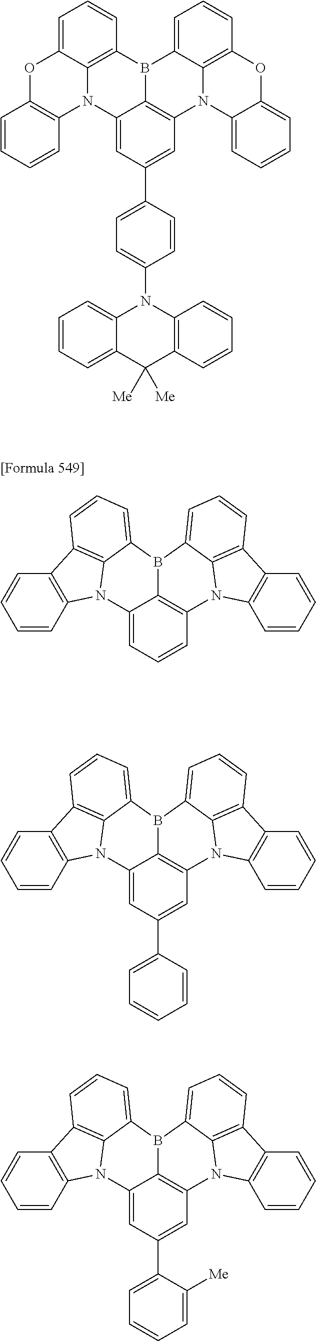 [SMILES: CC1(C)c2ccccc2N(c2ccc(-c3cc4c5c(c3)N3c6ccccc6Oc6cccc(c63)B5c3cccc5c3N4c3ccccc3O5)cc2)c2ccccc21 ; c1cc2c3c(c1)-n1c4ccccc4c4cccc(c41)B3c1cccc3c4ccccc4n-2c13 ; c1ccc(-c2cc3c4c(c2)-n2c5ccccc5c5cccc(c52)B4c2cccc4c5ccccc5n-3c24)cc1 ; Cc1ccccc1-c1cc2c3c(c1)-n1c4ccccc4c4cccc(c41)B3c1cccc3c4ccccc4n-2c13]

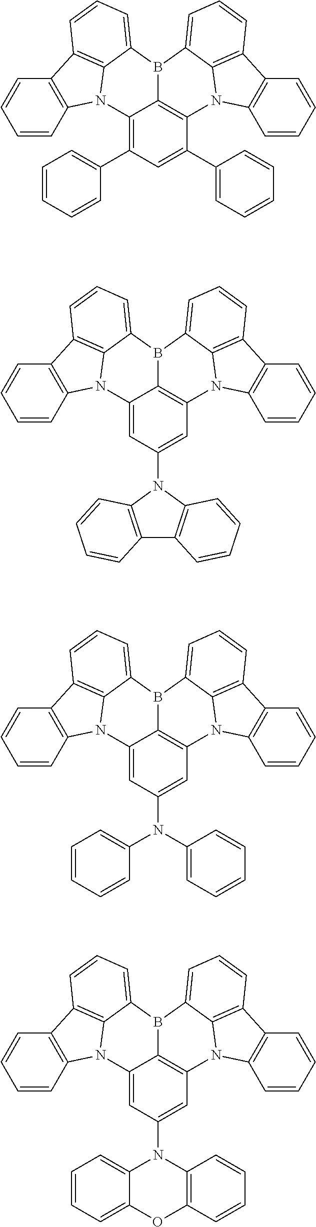 [SMILES: c1ccc(-c2cc(-c3ccccc3)c3c4c2-n2c5ccccc5c5cccc(c52)B4c2cccc4c5ccccc5n-3c24)cc1 ; c1ccc2c(c1)c1ccccc1n2-c1cc2c3c(c1)-n1c4ccccc4c4cccc(c41)B3c1cccc3c4ccccc4n-2c13 ; c1ccc(N(c2ccccc2)c2cc3c4c(c2)-n2c5ccccc5c5cccc(c52)B4c2cccc4c5ccccc5n-3c24)cc1 ; c1ccc2c(c1)Oc1ccccc1N2c1cc2c3c(c1)-n1c4ccccc4c4cccc(c41)B3c1cccc3c4ccccc4n-2c13]

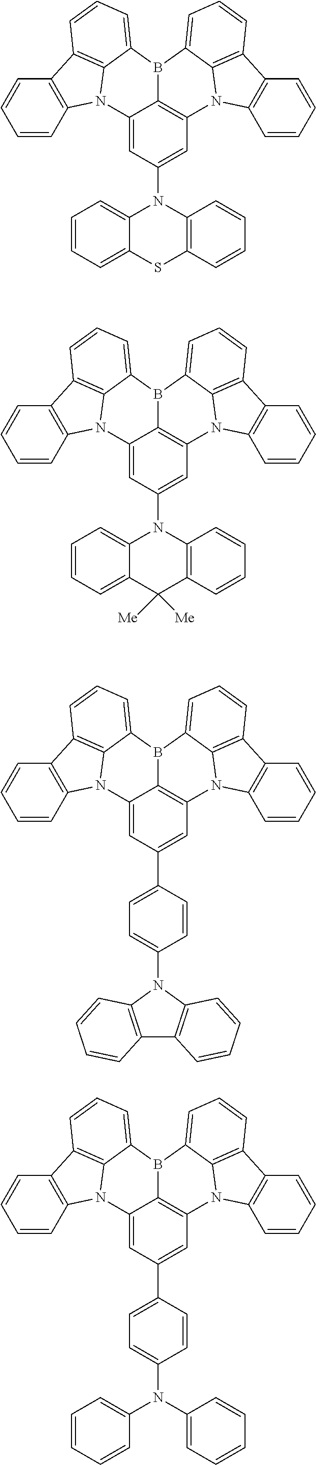

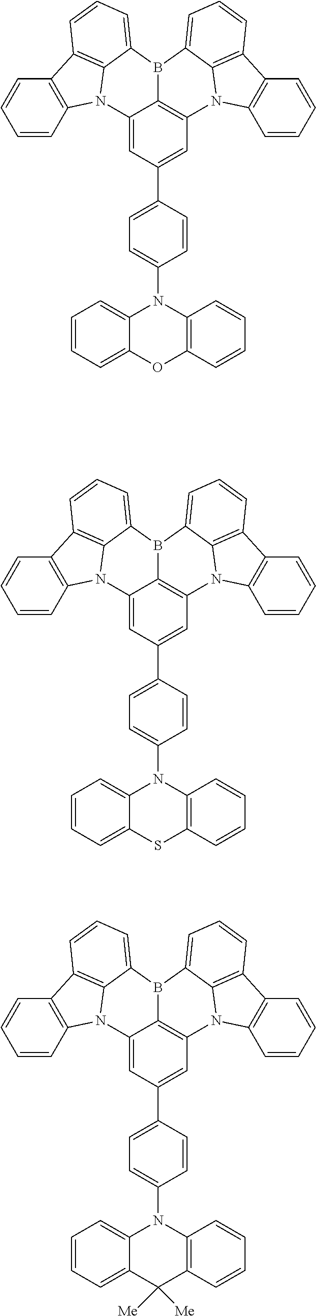 [SMILES: c1ccc2c(c1)Oc1ccccc1N2c1ccc(-c2cc3c4c(c2)-n2c5ccccc5c5cccc(c52)B4c2cccc4c5ccccc5n-3c24)cc1 ; c1ccc2c(c1)Sc1ccccc1N2c1ccc(-c2cc3c4c(c2)-n2c5ccccc5c5cccc(c52)B4c2cccc4c5ccccc5n-3c24)cc1 ; CC1(C)c2ccccc2N(c2ccc(-c3cc4c5c(c3)-n3c6ccccc6c6cccc(c63)B5c3cccc5c6ccccc6n-4c35)cc2)c2ccccc21]

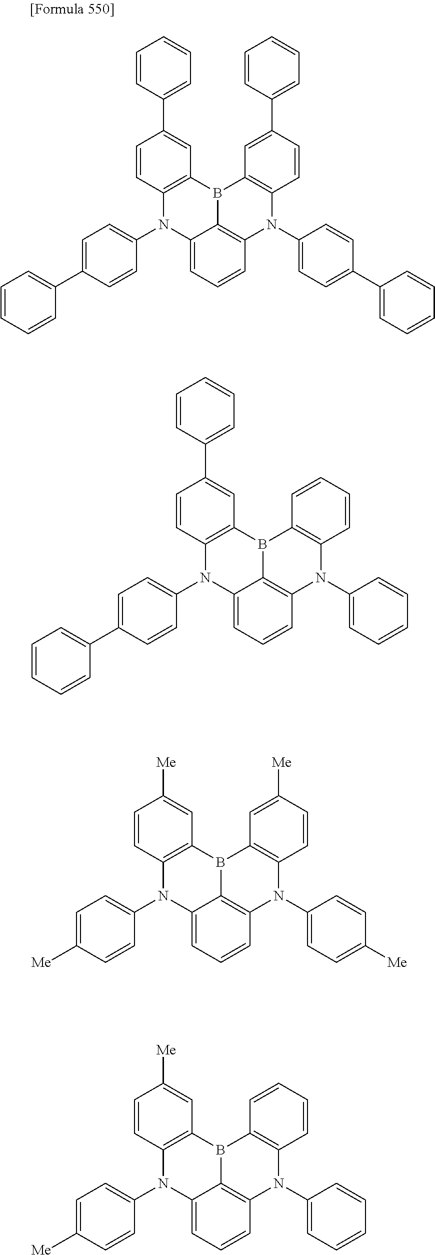 [SMILES: c1ccc(-c2ccc(N3c4ccc(-c5ccccc5)cc4B4c5cc(-c6ccccc6)ccc5N(c5ccc(-c6ccccc6)cc5)c5cccc3c54)cc2)cc1 ; c1ccc(-c2ccc(N3c4ccc(-c5ccccc5)cc4B4c5ccccc5N(c5ccccc5)c5cccc3c54)cc2)cc1 ; Cc1ccc(N2c3ccc(C)cc3B3c4cc(C)ccc4N(c4ccc(C)cc4)c4cccc2c43)cc1 ; Cc1ccc(N2c3ccc(C)cc3B3c4ccccc4N(c4ccccc4)c4cccc2c43)cc1]

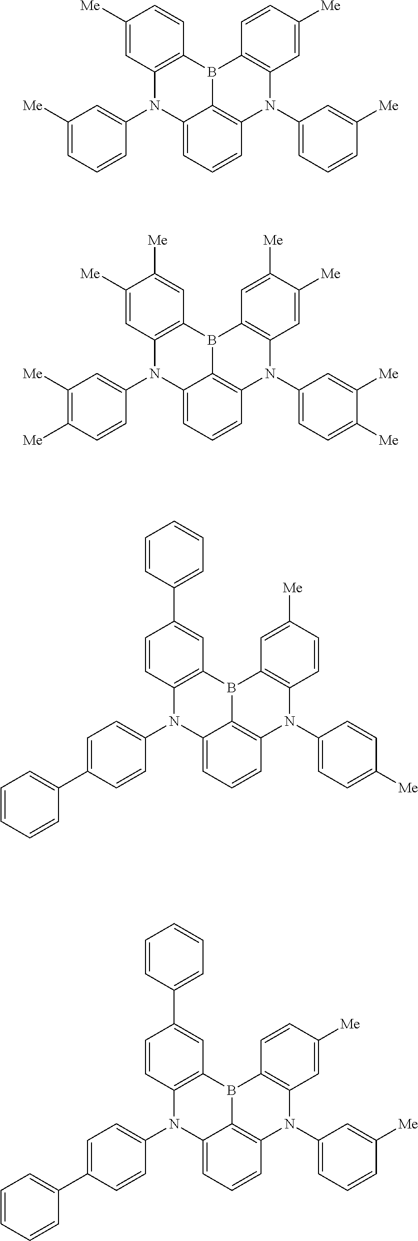 [SMILES: Cc1cccc(N2c3cc(C)ccc3B3c4ccc(C)cc4N(c4cccc(C)c4)c4cccc2c43)c1 ; Cc1ccc(N2c3cc(C)c(C)cc3B3c4cc(C)c(C)cc4N(c4ccc(C)c(C)c4)c4cccc2c43)cc1C ; Cc1ccc(N2c3ccc(C)cc3B3c4cc(-c5ccccc5)ccc4N(c4ccc(-c5ccccc5)cc4)c4cccc2c43)cc1 ; Cc1cccc(N2c3cc(C)ccc3B3c4cc(-c5ccccc5)ccc4N(c4ccc(-c5ccccc5)cc4)c4cccc2c43)c1]

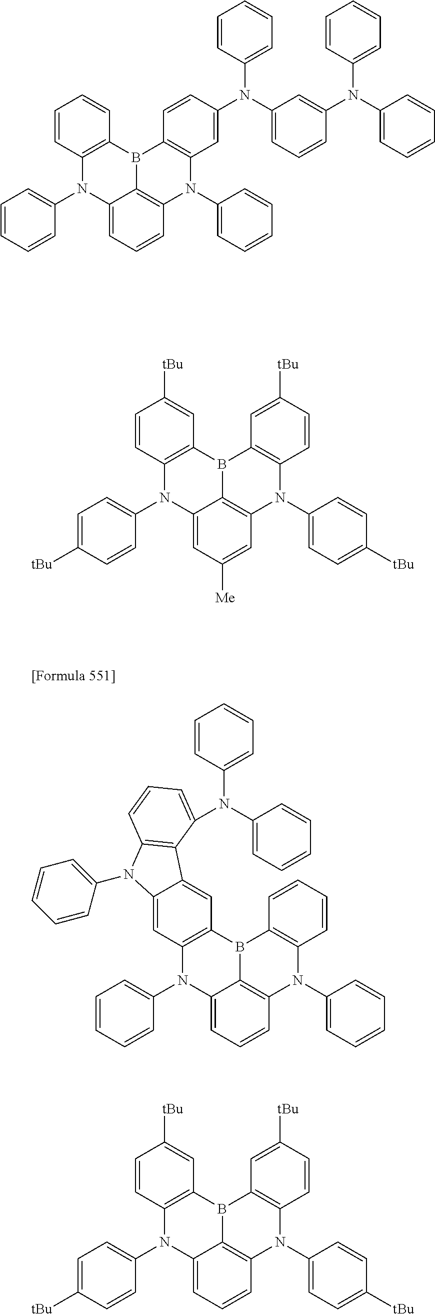 [SMILES: c1ccc(N(c2ccccc2)c2cccc(N(c3ccccc3)c3ccc4c(c3)N(c3ccccc3)c3cccc5c3B4c3ccccc3N5c3ccccc3)c2)cc1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; c1ccc(N2c3ccccc3B3c4cc5c6c(N(c7ccccc7)c7ccccc7)cccc6n(-c6ccccc6)c5cc4N(c4ccccc4)c4cccc2c43)cc1 ; CC(C)(C)c1ccc(N2c3ccc(C(C)(C)C)cc3B3c4cc(C(C)(C)C)ccc4N(c4ccc(C(C)(C)C)cc4)c4cccc2c43)cc1]

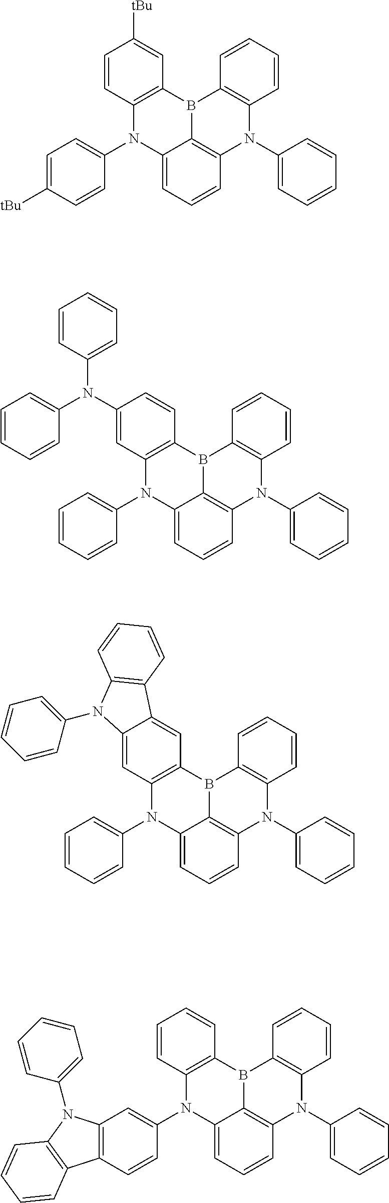 [SMILES: CC(C)(C)c1ccc(N2c3ccc(C(C)(C)C)cc3B3c4ccccc4N(c4ccccc4)c4cccc2c43)cc1 ; c1ccc(N(c2ccccc2)c2ccc3c(c2)N(c2ccccc2)c2cccc4c2B3c2ccccc2N4c2ccccc2)cc1 ; c1ccc(N2c3ccccc3B3c4cc5c6ccccc6n(-c6ccccc6)c5cc4N(c4ccccc4)c4cccc2c43)cc1 ; c1ccc(N2c3ccccc3B3c4ccccc4N(c4ccc5c6ccccc6n(-c6ccccc6)c5c4)c4cccc2c43)cc1]

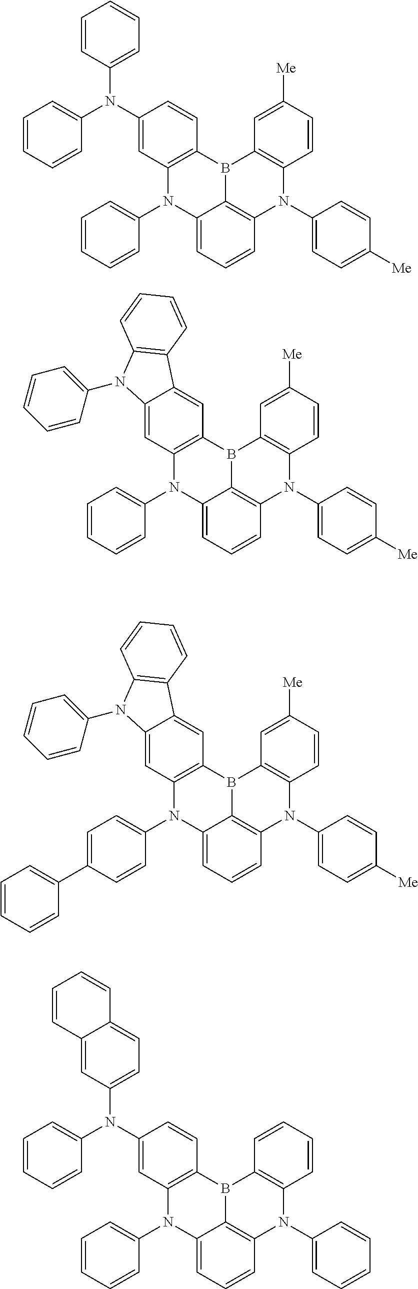

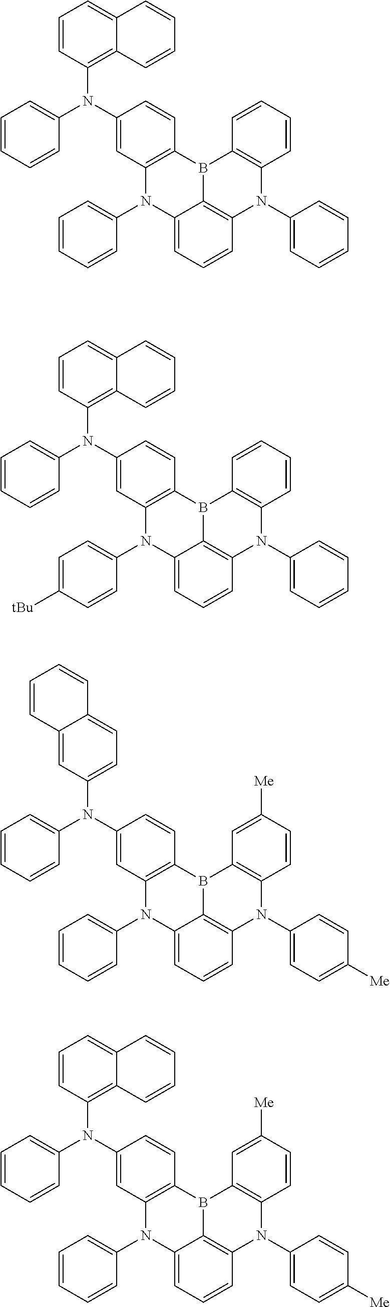 [SMILES: c1ccc(N2c3ccccc3B3c4ccc(N(c5ccccc5)c5cccc6ccccc56)cc4N(c4ccccc4)c4cccc2c43)cc1 ; CC(C)(C)c1ccc(N2c3cc(N(c4ccccc4)c4cccc5ccccc45)ccc3B3c4ccccc4N(c4ccccc4)c4cccc2c43)cc1 ; Cc1ccc(N2c3ccc(C)cc3B3c4ccc(N(c5ccccc5)c5ccc6ccccc6c5)cc4N(c4ccccc4)c4cccc2c43)cc1 ; Cc1ccc(N2c3ccc(C)cc3B3c4ccc(N(c5ccccc5)c5cccc6ccccc56)cc4N(c4ccccc4)c4cccc2c43)cc1]

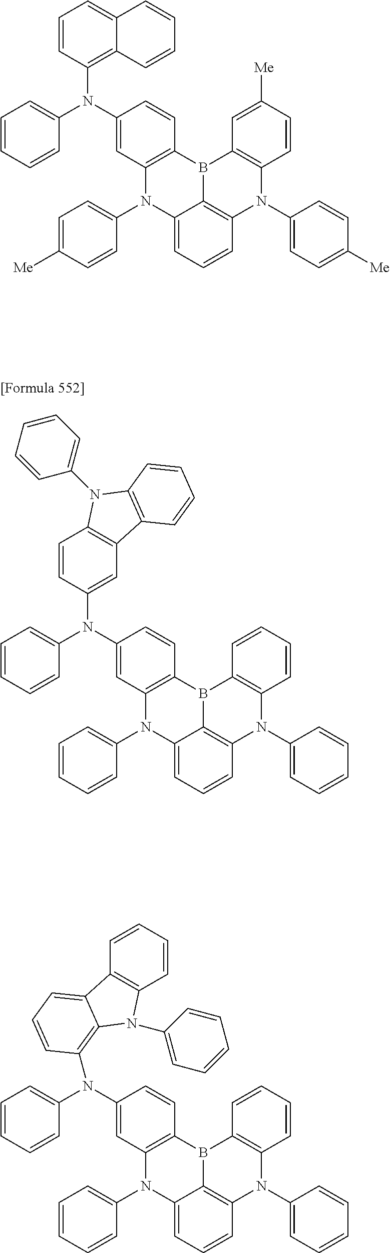 [SMILES: Cc1ccc(N2c3ccc(C)cc3B3c4ccc(N(c5ccccc5)c5cccc6ccccc56)cc4N(c4ccc(C)cc4)c4cccc2c43)cc1 ; c1ccc(N(c2ccc3c(c2)N(c2ccccc2)c2cccc4c2B3c2ccccc2N4c2ccccc2)c2ccc3c(c2)c2ccccc2n3-c2ccccc2)cc1 ; c1ccc(N2c3ccccc3B3c4ccc(N(c5ccccc5)c5cccc6c7ccccc7n(-c7ccccc7)c56)cc4N(c4ccccc4)c4cccc2c43)cc1]

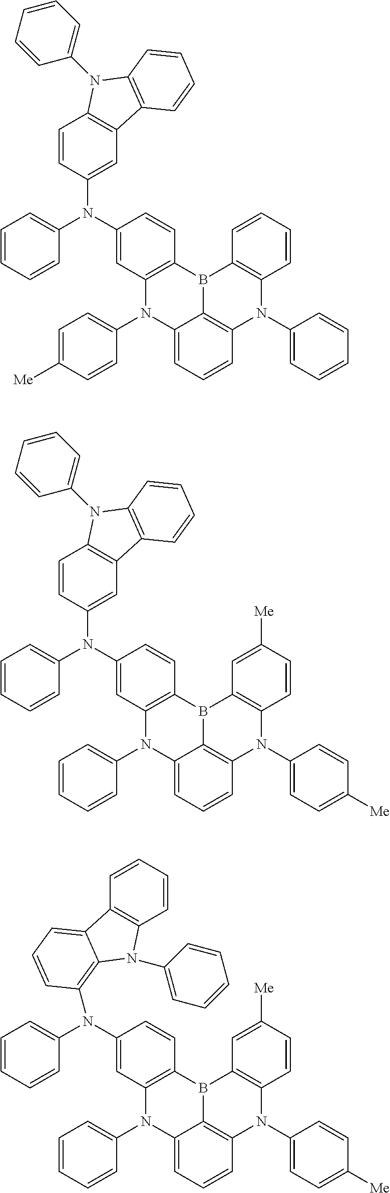 [SMILES: Cc1ccc(N2c3cc(N(c4ccccc4)c4ccc5c(c4)c4ccccc4n5-c4ccccc4)ccc3B3c4ccccc4N(c4ccccc4)c4cccc2c43)cc1 ; Cc1ccc(N2c3ccc(C)cc3B3c4ccc(N(c5ccccc5)c5ccc6c(c5)c5ccccc5n6-c5ccccc5)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccccc4)c4cccc2c43)cc1]

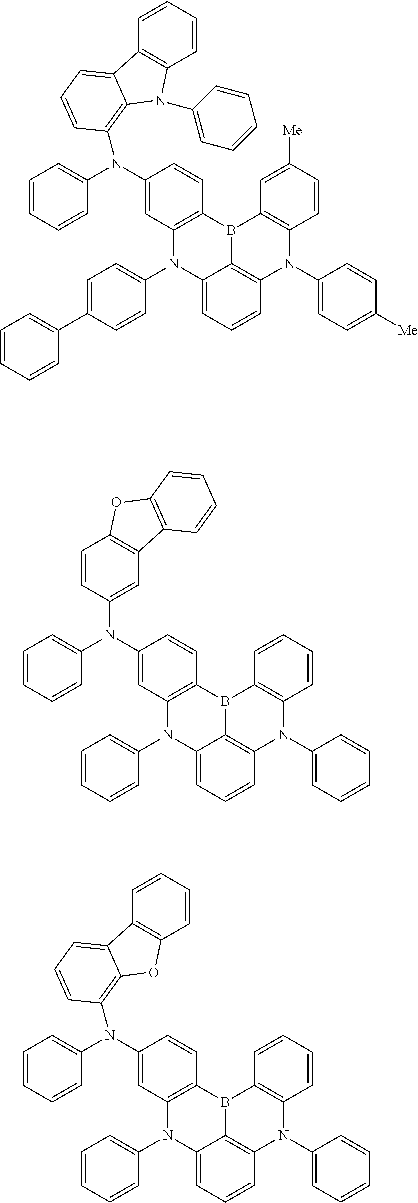 [SMILES: Cc1ccc(N2c3ccc(C)cc3B3c4ccc(N(c5ccccc5)c5cccc6c7ccccc7n(-c7ccccc7)c56)cc4N(c4ccc(-c5ccccc5)cc4)c4cccc2c43)cc1 ; c1ccc(N(c2ccc3c(c2)N(c2ccccc2)c2cccc4c2B3c2ccccc2N4c2ccccc2)c2ccc3oc4ccccc4c3c2)cc1 ; c1ccc(N2c3ccccc3B3c4ccc(N(c5ccccc5)c5cccc6c5oc5ccccc56)cc4N(c4ccccc4)c4cccc2c43)cc1]

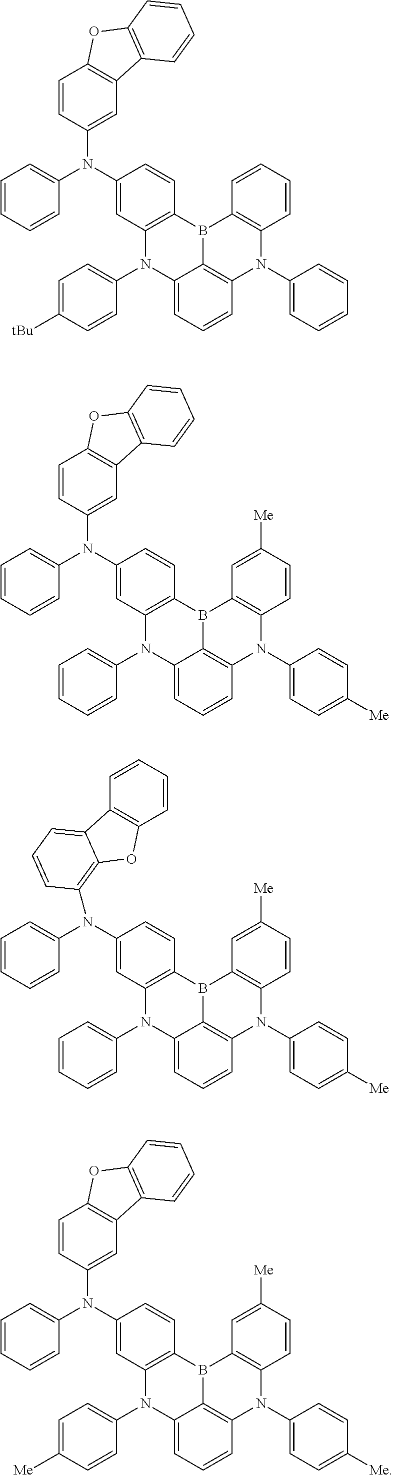 [SMILES: CC(C)(C)c1ccc(N2c3cc(N(c4ccccc4)c4ccc5oc6ccccc6c5c4)ccc3B3c4ccccc4N(c4ccccc4)c4cccc2c43)cc1 ; Cc1ccc(N2c3ccc(C)cc3B3c4ccc(N(c5ccccc5)c5ccc6oc7ccccc7c6c5)cc4N(c4ccccc4)c4cccc2c43)cc1 ; Cc1ccc(N2c3ccc(C)cc3B3c4ccc(N(c5ccccc5)c5cccc6c5oc5ccccc56)cc4N(c4ccccc4)c4cccc2c43)cc1 ; Cc1ccc(N2c3ccc(C)cc3B3c4ccc(N(c5ccccc5)c5ccc6oc7ccccc7c6c5)cc4N(c4ccc(C)cc4)c4cccc2c43)cc1]

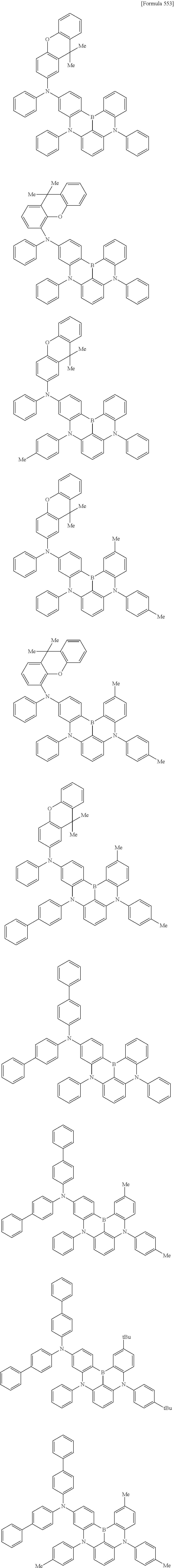

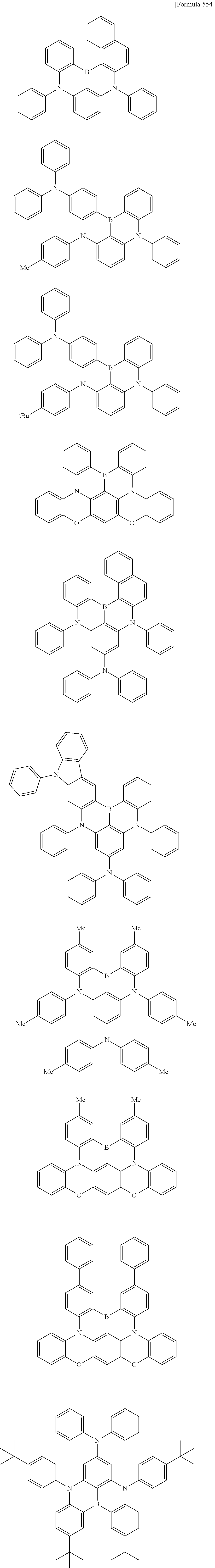

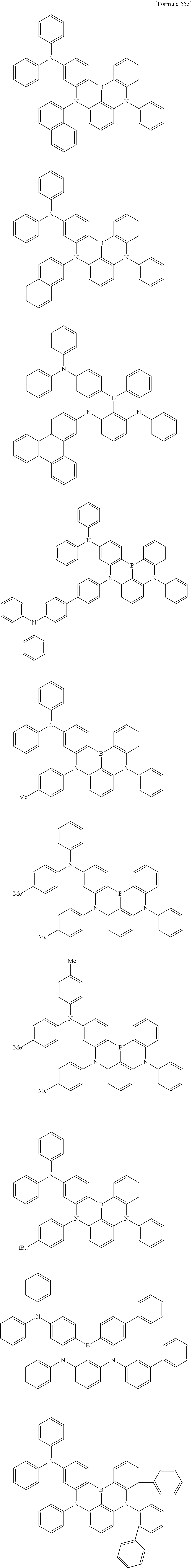

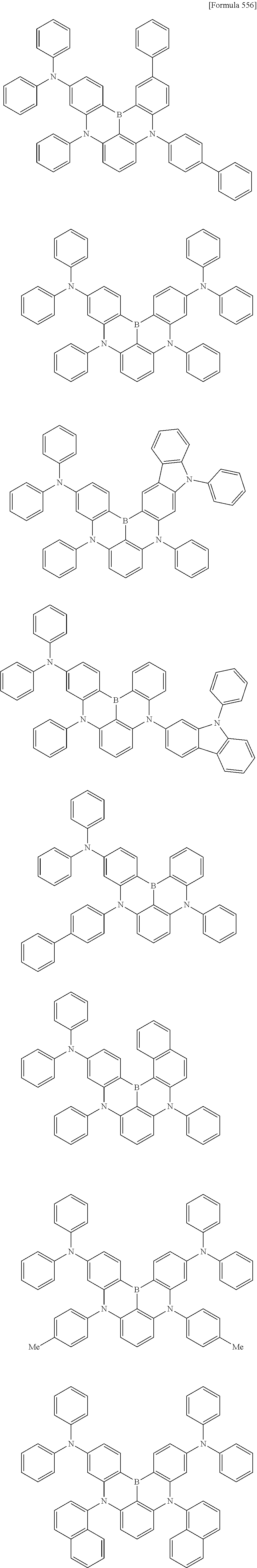

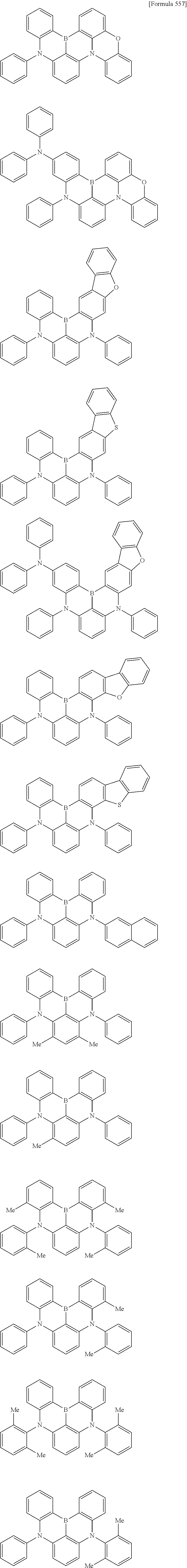

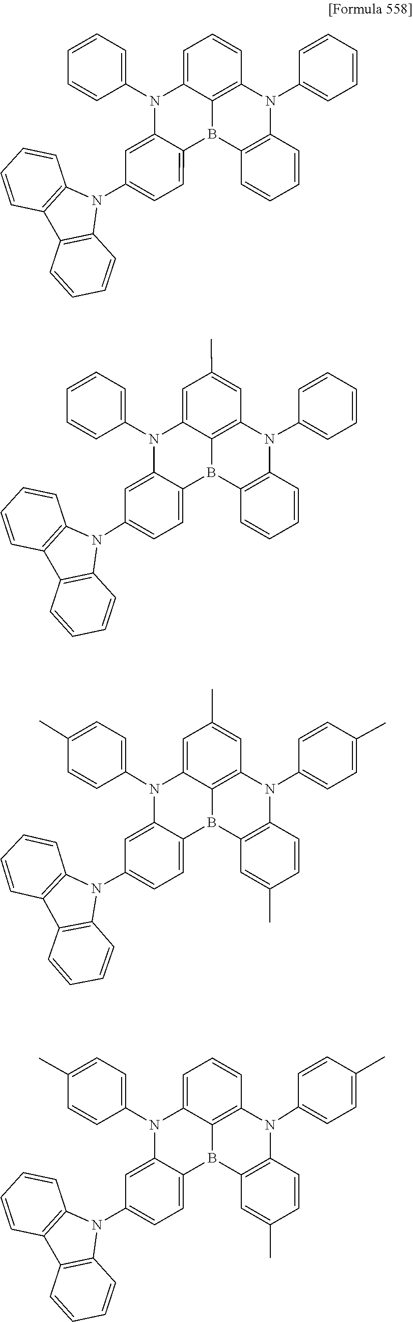 [SMILES: c1ccc(N2c3ccccc3B3c4ccc(-n5c6ccccc6c6ccccc65)cc4N(c4ccccc4)c4cccc2c43)cc1 ; Cc1cc2c3c(c1)N(c1ccccc1)c1cc(-n4c5ccccc5c5ccccc54)ccc1B3c1ccccc1N2c1ccccc1 ; Cc1ccc(N2c3ccc(C)cc3B3c4ccc(-n5c6ccccc6c6ccccc65)cc4N(c4ccc(C)cc4)c4cc(C)cc2c43)cc1 ; Cc1ccc(N2c3ccc(C)cc3B3c4ccc(-n5c6ccccc6c6ccccc65)cc4N(c4ccc(C)cc4)c4cccc2c43)cc1]

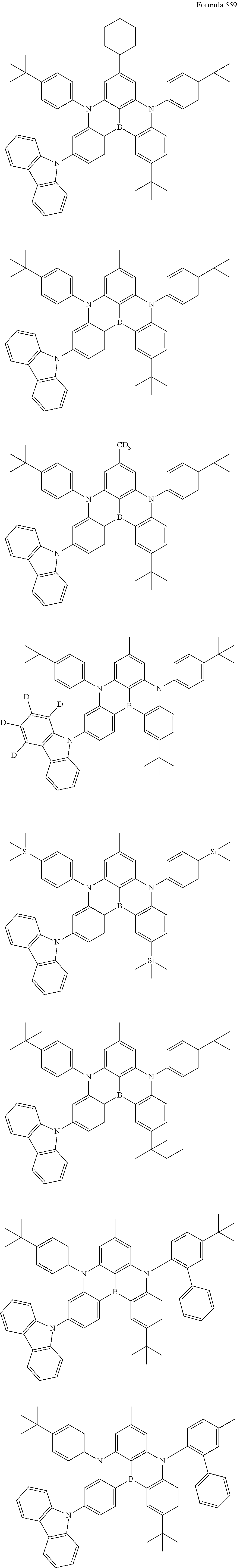

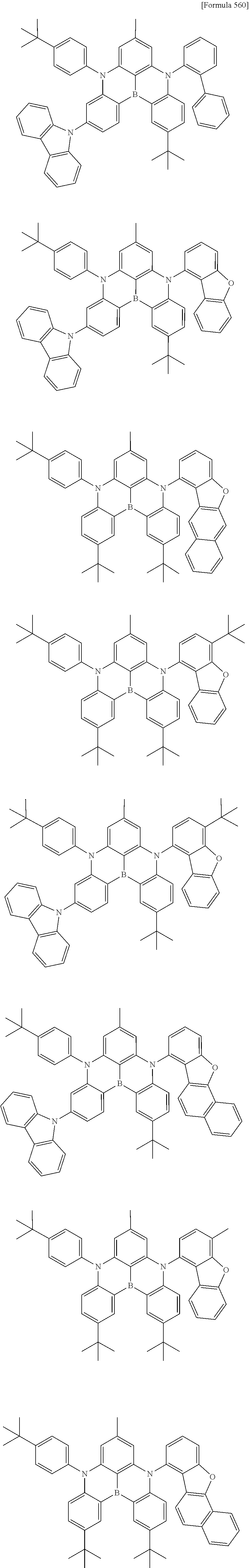

### Compound Represented by Formula (7)

The compound represented by the formula (7) will be described below.

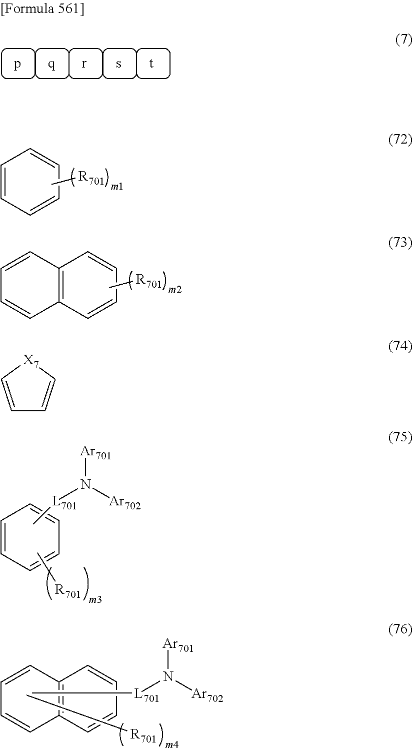

In the formula (7):
r ring is a ring represented by the formula (72) or the formula (73), the r ring being fused with adjacent ring(s) at any position(s);
q ring and s ring are each independently a ring represented by the formula (74) and fused with adjacent ring(s) at any position(s);
p ring and t ring are each independently a structure represented by the formula (75) or the formula (76) and fused with adjacent ring(s) at any position(s);
X₇ is an oxygen atom, a sulfur atom, or NR₇₀₂;
when a plurality of R₇₀₁ are present, adjacent ones of the plurality of R₇₀₁ are mutually bonded to form a substituted or unsubstituted monocyclic ring, mutually bonded to form a substituted or unsubstituted fused ring, or not mutually bonded;
R₇₀₁ and R₇₀₂ forming neither the monocyclic ring nor the fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
Ar₇₀₁ and Ar₇₀₂ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
L₇₀₁ is a substituted or unsubstituted alkylene group having 1 to 50 carbon atoms, a substituted or unsubstituted alkenylene group having 2 to 50 carbon atoms, a substituted or unsubstituted alkynylene group having 2 to 50 carbon atoms, a substituted or unsubstituted cycloalkylene group having 3 to 50 ring carbon atoms, a substituted or unsubstituted arylene group having 6 to 50 ring carbon atoms, or a substituted or unsubstituted divalent heterocyclic group having 5 to 50 ring atoms;
m1 is 0, 1, or 2;
m2 is 0, 1, 2, 3, or 4;
m3 is each independently 0, 1, 2, or 3;
m4 is each independently 0, 1, 2, 3, 4, or 5;
when a plurality of R₇₀₁ are present, the plurality of R₇₀₁ are mutually the same or different;
when a plurality of X₇ are present, the plurality of X₇ are mutually the same or different;
when a plurality of R₇₀₂ are present, the plurality of R₇₀₂ are mutually the same or different;
when a plurality of Ar₇₀₁ are present, the plurality of Ar₇₀₁ are mutually the same or different;
when a plurality of Ar₇₀₂ are present, the plurality of Ar₇₀₂ are mutually the same or different; and
when a plurality of L₇₀₁ are present, the plurality of L₇₀₁ are mutually the same or different.

In the formula (7), each of the p ring, q ring, r ring, s ring, and t ring is fused with an adjacent ring(s) sharing two carbon atoms. The fused position and orientation are not limited but may be defined as required.

In an exemplary embodiment, in the formula (72) or the formula (73) representing the r ring, m1=0 or m2=0 is satisfied.

In an exemplary embodiment, the compound represented by the formula (7) is represented by any one of formulae (71-1) to (71-6) below.

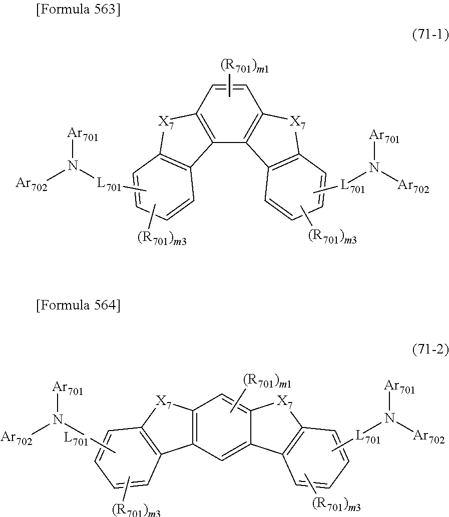

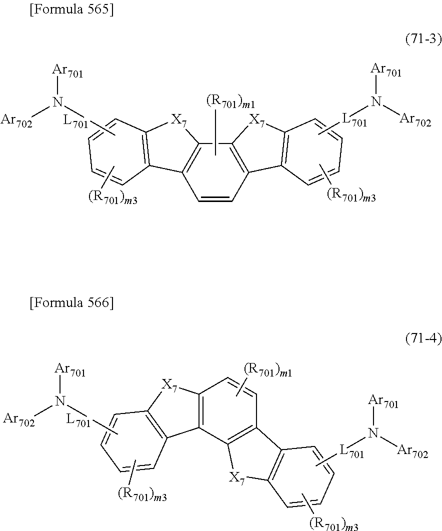

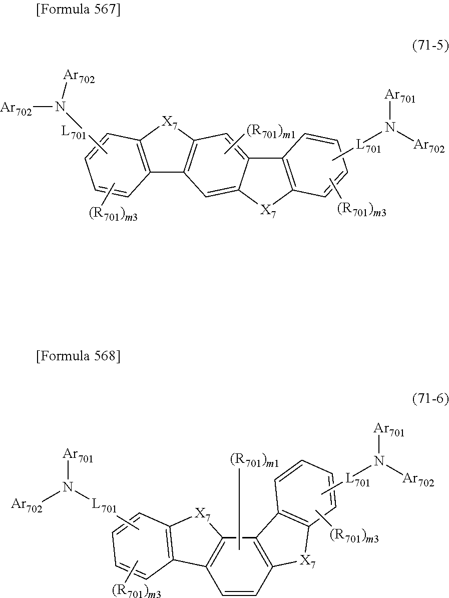

In the formulae (71-1) to (71-6), R₇₀₁, X₇, Ar₇₀₁, Ar₇₀₂, L₇₀₁, m1 and m3 respectively represent the same as R₇₀₁, X₇, Ar₇₀₁, Ar₇₀₂, L₇₀₁, m1 and m3 in the formula (7).

In an exemplary embodiment, the compound represented by the formula (7) is represented by any one of formulae (71-11) to (71-13) below.

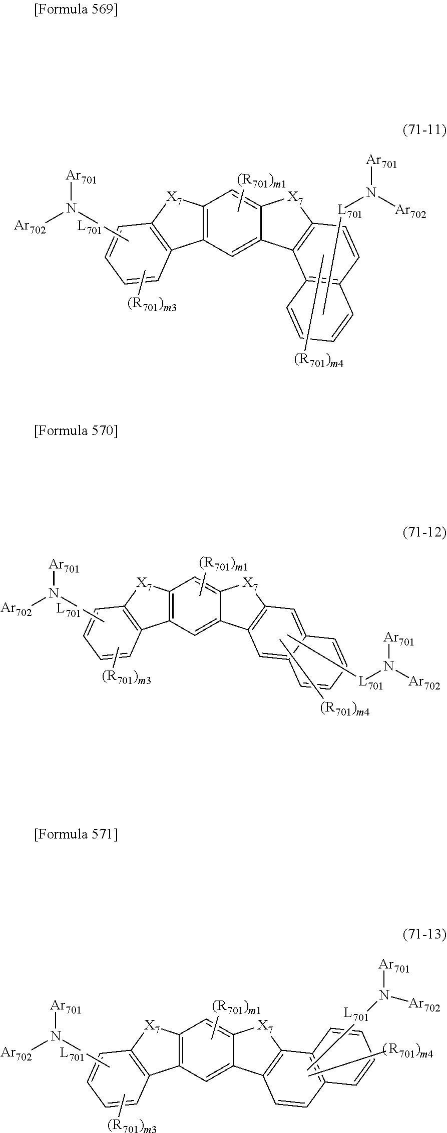

In the formulae (71-11) to (71-13), R₇₀₁, X₇, Ar₇₀₁, Ar₇₀₂, L₇₀₁, m1, m3 and m4 respectively represent the same as R₇₀₁, X₇, Ar₇₀₁, Ar₇₀₂, L₇₀₁, m1, m3 and m4 in the formula (7).

In an exemplary embodiment, the compound represented by the formula (7) is represented by any one of formulae (71-21) to (71-25) below.

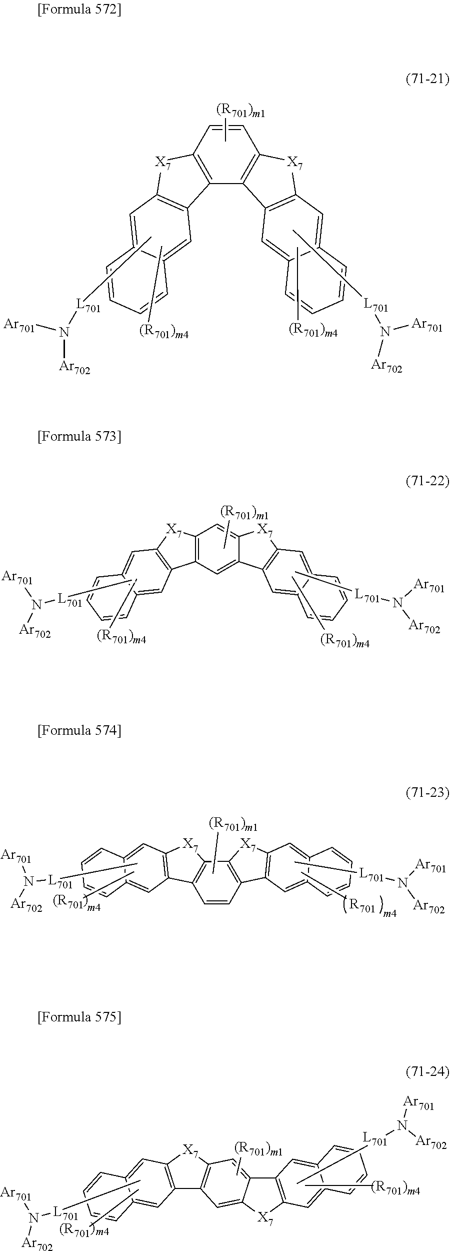

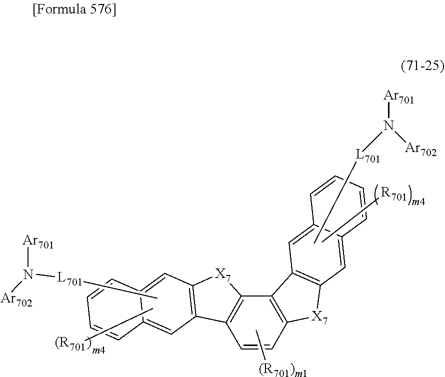

In the formulae (71-21) to (71-25), R₇₀₁, X₇, Ar₇₀₁, Ar₇₀₂, L₇₀₁, m1 and m4 respectively represent the same as R₇₀₁, X₇, Ar₇₀₁, Ar₇₀₂, L₇₀₁, m1 and m4 in the formula (7).

In an exemplary embodiment, the compound represented by the formula (7) is represented by any one of formulae (71-31) to (71-33) below.

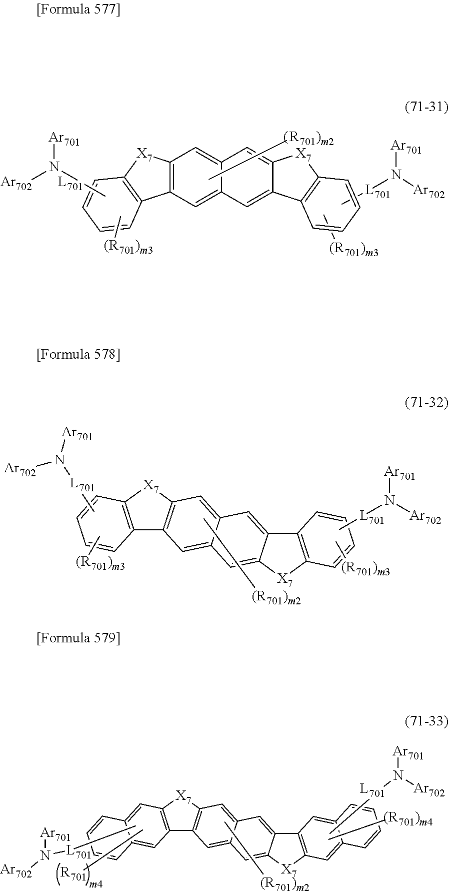

In the formulae (71-31) to (71-33), R₇₀₁, X₇, Ar₇₀₁, Ar₇₀₂, L₇₀₁, and m2 to m4 respectively represent the same as R₇₀₁, X₇, Ar₇₀₁, Ar₇₀₂, L₇₀₁, and m2 to m4 in the formula (7).

In an exemplary embodiment, Ar₇₀₁ and Ar₇₀₂ are each independently a substituted or unsubstituted aryl group having 6 to 50 ring carbon atoms.

In an exemplary embodiment, one of Ar₇₀₁ and Ar₇₀₂ is a substituted or unsubstituted aryl group having 6 to 50 ring carbon atoms, and the other of Ar₇₀₁ and

Ar₇₀₂ is a substituted or unsubstituted heterocyclic group having 5 to 50 ring atoms.

Specific examples of the compound represented by the formula (7) include compounds shown below.

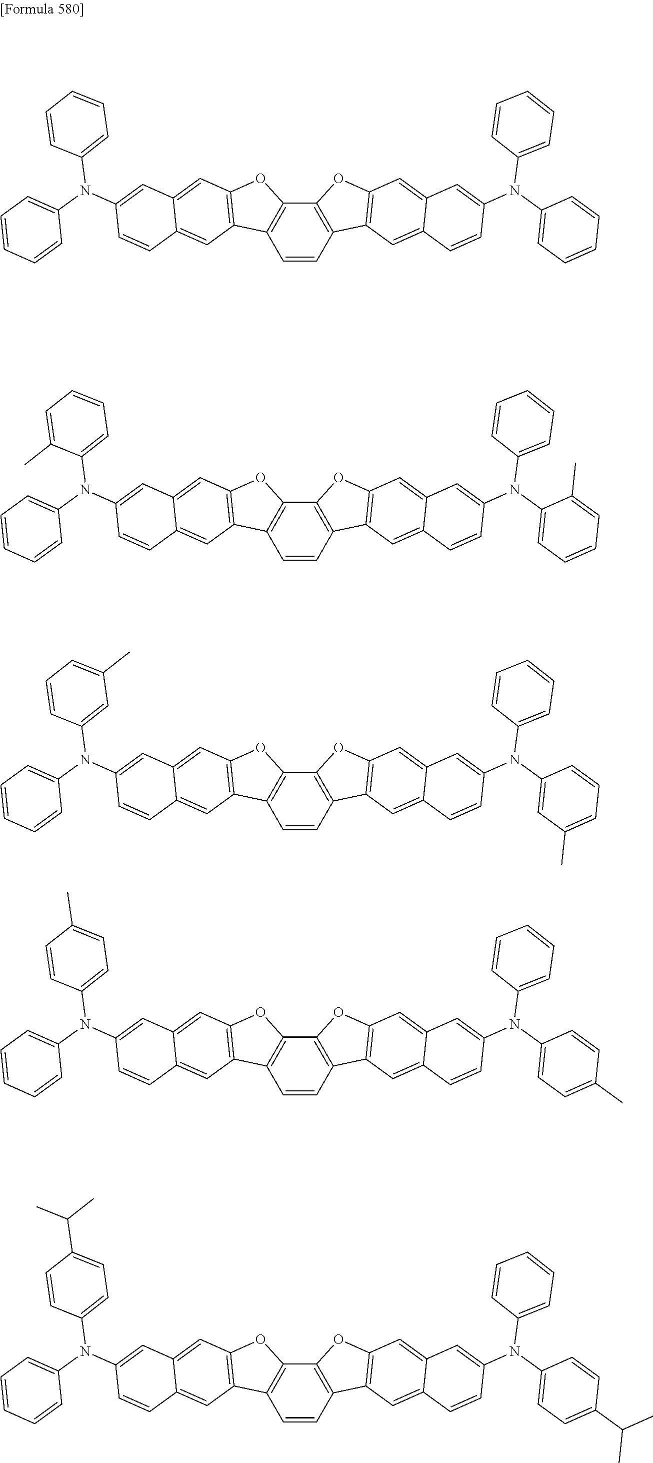 [SMILES: c1ccc(N(c2ccccc2)c2ccc3cc4c(cc3c2)oc2c4ccc3c4cc5ccc(N(c6ccccc6)c6ccccc6)cc5cc4oc32)cc1 ; Cc1ccccc1N(c1ccccc1)c1ccc2cc3c(cc2c1)oc1c3ccc2c3cc4ccc(N(c5ccccc5)c5ccccc5C)cc4cc3oc21 ; Cc1cccc(N(c2ccccc2)c2ccc3cc4c(cc3c2)oc2c4ccc3c4cc5ccc(N(c6ccccc6)c6cccc(C)c6)cc5cc4oc32)c1 ; Cc1ccc(N(c2ccccc2)c2ccc3cc4c(cc3c2)oc2c4ccc3c4cc5ccc(N(c6ccccc6)c6ccc(C)cc6)cc5cc4oc32)cc1 ; CC(C)c1ccc(N(c2ccccc2)c2ccc3cc4c(cc3c2)oc2c4ccc3c4cc5ccc(N(c6ccccc6)c6ccc(C(C)C)cc6)cc5cc4oc32)cc1]

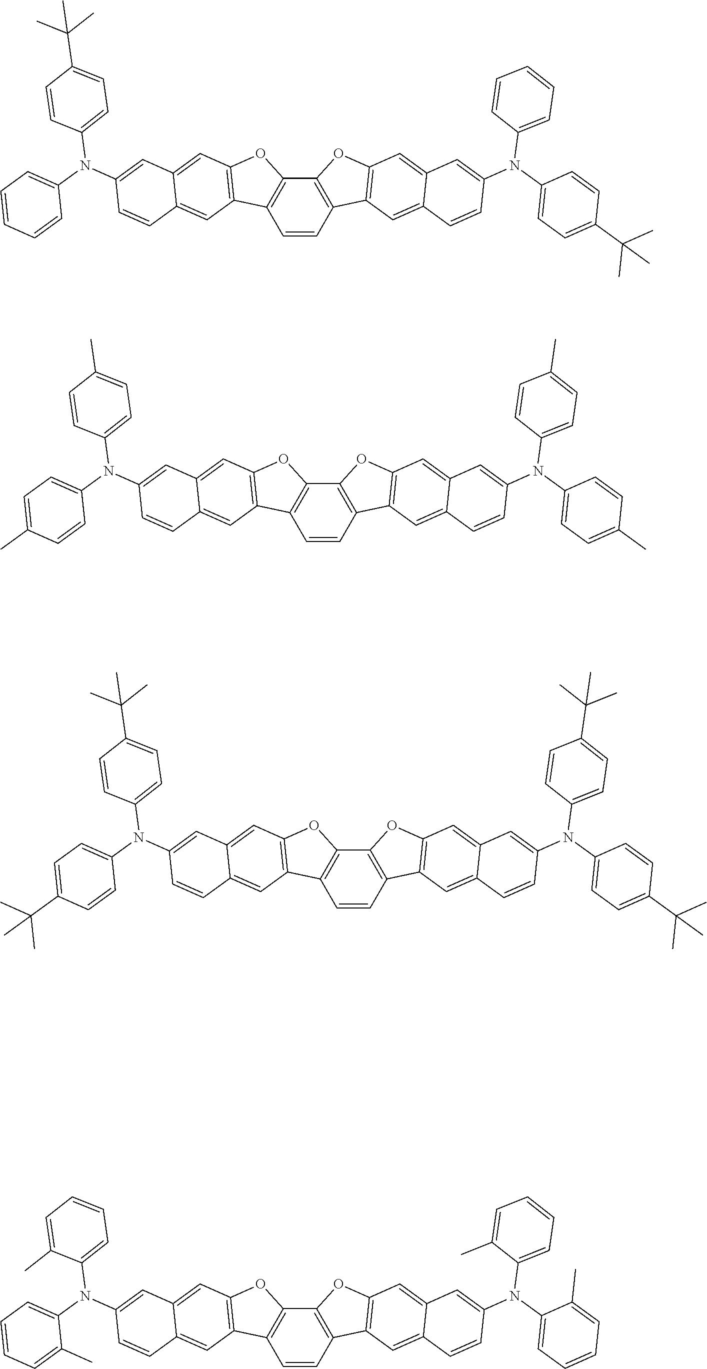

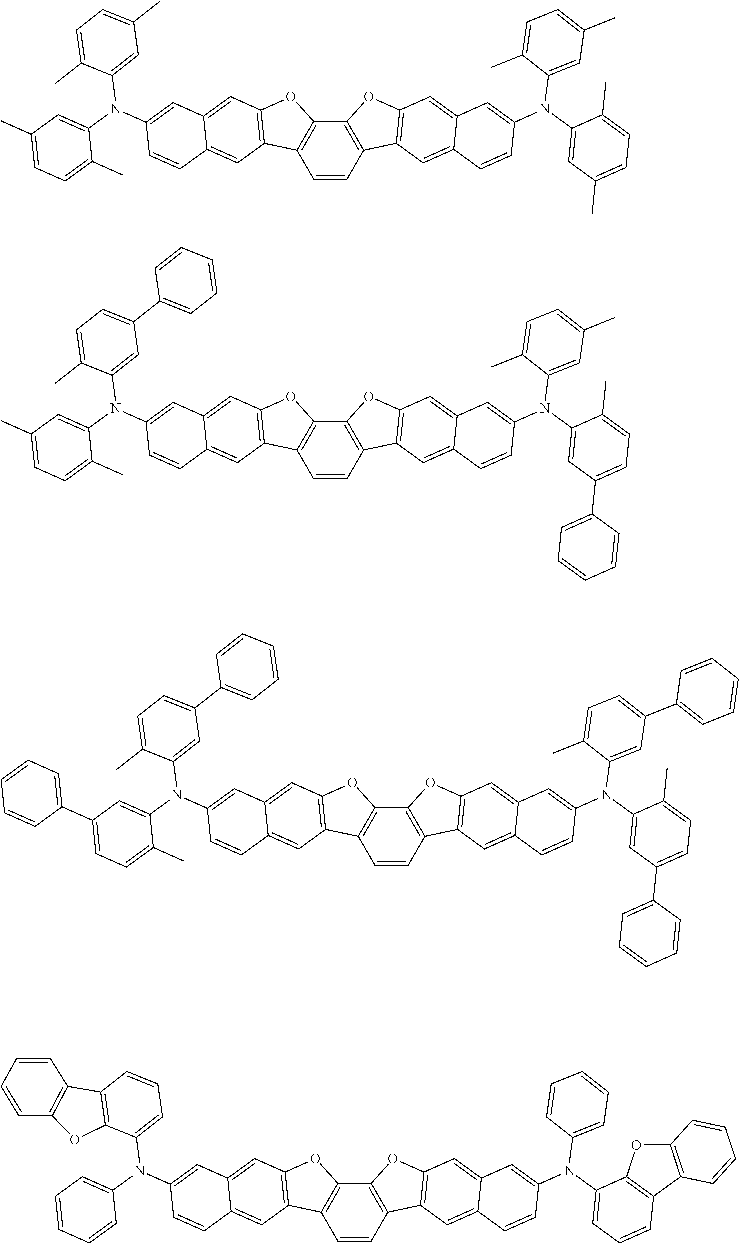 [SMILES: Cc1ccc(C)c(N(c2ccc3cc4c(cc3c2)oc2c4ccc3c4cc5ccc(N(c6cc(C)ccc6C)c6cc(C)ccc6C)cc5cc4oc32)c2cc(C)ccc2C)c1 ; Cc1ccc(C)c(N(c2ccc3cc4c(cc3c2)oc2c4ccc3c4cc5ccc(N(c6cc(C)ccc6C)c6cc(-c7ccccc7)ccc6C)cc5cc4oc32)c2cc(-c3ccccc3)ccc2C)c1 ; Cc1ccc(-c2ccccc2)cc1N(c1ccc2cc3c(cc2c1)oc1c3ccc2c3cc4ccc(N(c5cc(-c6ccccc6)ccc5C)c5cc(-c6ccccc6)ccc5C)cc4cc3oc21)c1cc(-c2ccccc2)ccc1C ; c1ccc(N(c2ccc3cc4c(cc3c2)oc2c4ccc3c4cc5ccc(N(c6ccccc6)c6cccc7c6oc6ccccc67)cc5cc4oc32)c2cccc3c2oc2ccccc23)cc1]

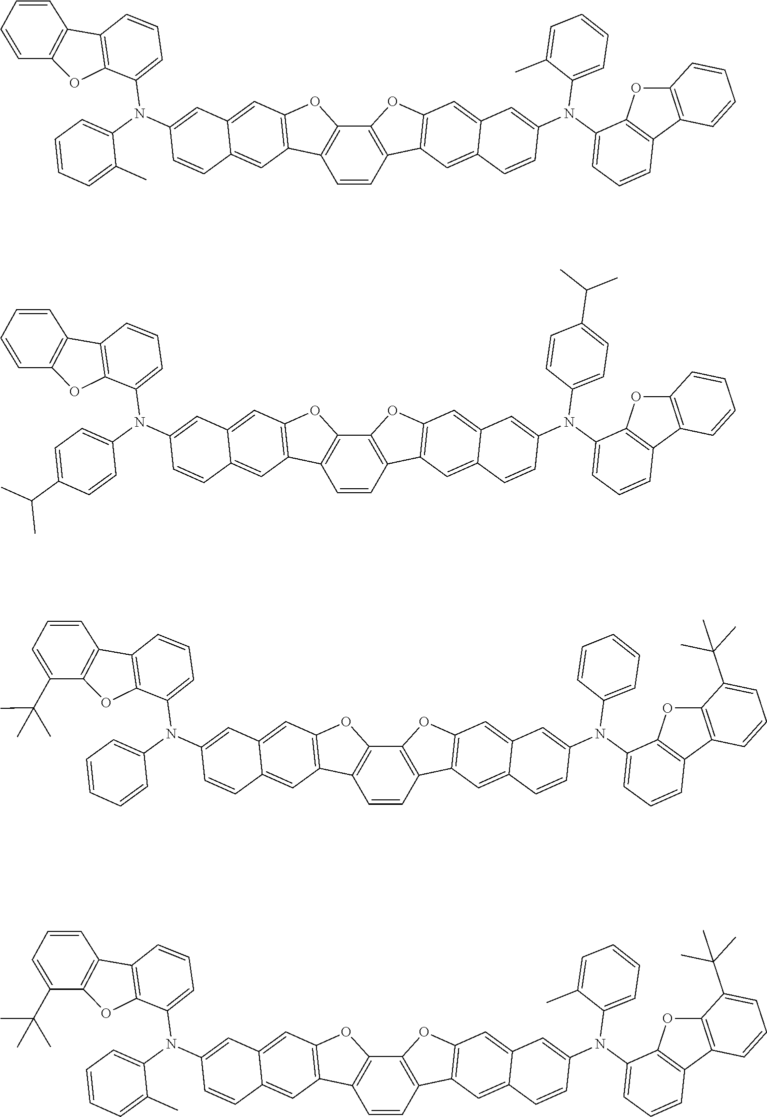 [SMILES: Cc1ccccc1N(c1ccc2cc3c(cc2c1)oc1c3ccc2c3cc4ccc(N(c5ccccc5C)c5cccc6c5oc5ccccc56)cc4cc3oc21)c1cccc2c1oc1ccccc12 ; CC(C)c1ccc(N(c2ccc3cc4c(cc3c2)oc2c4ccc3c4cc5ccc(N(c6ccc(C(C)C)cc6)c6cccc7c6oc6ccccc67)cc5cc4oc32)c2cccc3c2oc2ccccc23)cc1 ; CC(C)(C)c1cccc2c1oc1c(N(c3ccccc3)c3ccc4cc5c(cc4c3)oc3c5ccc4c5cc6ccc(N(c7ccccc7)c7cccc8c7oc7c(C(C)(C)C)cccc78)cc6cc5oc43)cccc12 ; Cc1ccccc1N(c1ccc2cc3c(cc2c1)oc1c3ccc2c3cc4ccc(N(c5ccccc5C)c5cccc6c5oc5c(C(C)(C)C)cccc56)cc4cc3oc21)c1cccc2c1oc1c(C(C)(C)C)cccc12]

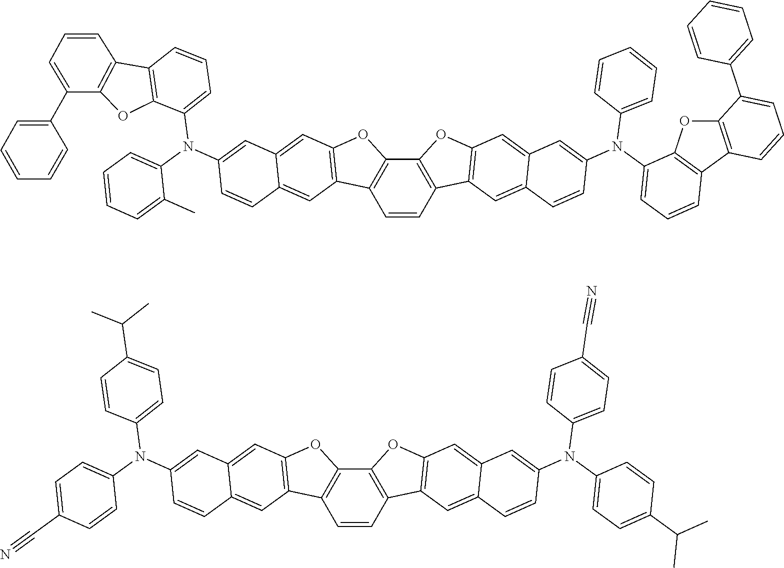 [SMILES: Cc1ccccc1N(c1ccc2cc3c(cc2c1)oc1c3ccc2c3cc4ccc(N(c5ccccc5)c5cccc6c5oc5c(-c7ccccc7)cccc56)cc4cc3oc21)c1cccc2c1oc1c(-c3ccccc3)cccc12 ; CC(C)c1ccc(N(c2ccc(C#N)cc2)c2ccc3cc4c(cc3c2)oc2c4ccc3c4cc5ccc(N(c6ccc(C#N)cc6)c6ccc(C(C)C)cc6)cc5cc4oc32)cc1]

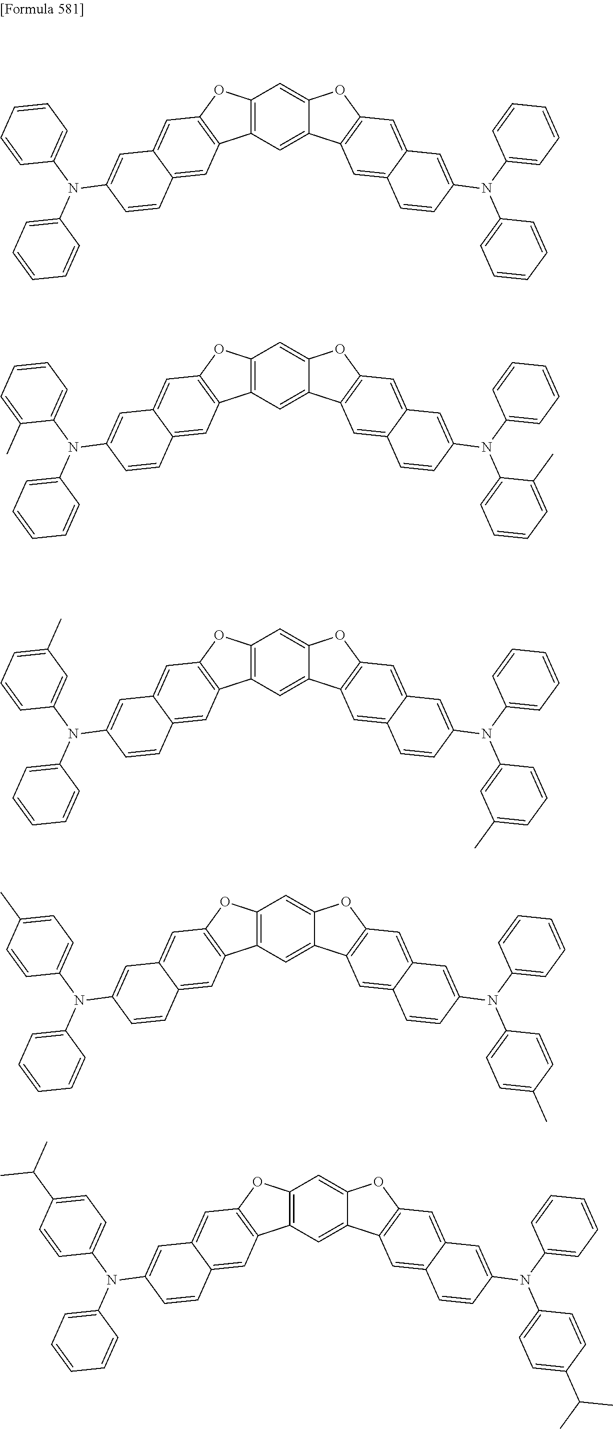 [SMILES: c1ccc(N(c2ccccc2)c2ccc3cc4c(cc3c2)oc2cc3oc5cc6cc(N(c7ccccc7)c7ccccc7)ccc6cc5c3cc24)cc1 ; Cc1ccccc1N(c1ccccc1)c1ccc2cc3c(cc2c1)oc1cc2oc4cc5cc(N(c6ccccc6)c6ccccc6C)ccc5cc4c2cc13 ; Cc1cccc(N(c2ccccc2)c2ccc3cc4c(cc3c2)oc2cc3oc5cc6cc(N(c7ccccc7)c7cccc(C)c7)ccc6cc5c3cc24)c1 ; Cc1ccc(N(c2ccccc2)c2ccc3cc4c(cc3c2)oc2cc3oc5cc6cc(N(c7ccccc7)c7ccc(C)cc7)ccc6cc5c3cc24)cc1 ; CC(C)c1ccc(N(c2ccccc2)c2ccc3cc4c(cc3c2)oc2cc3oc5cc6cc(N(c7ccccc7)c7ccc(C(C)C)cc7)ccc6cc5c3cc24)cc1]

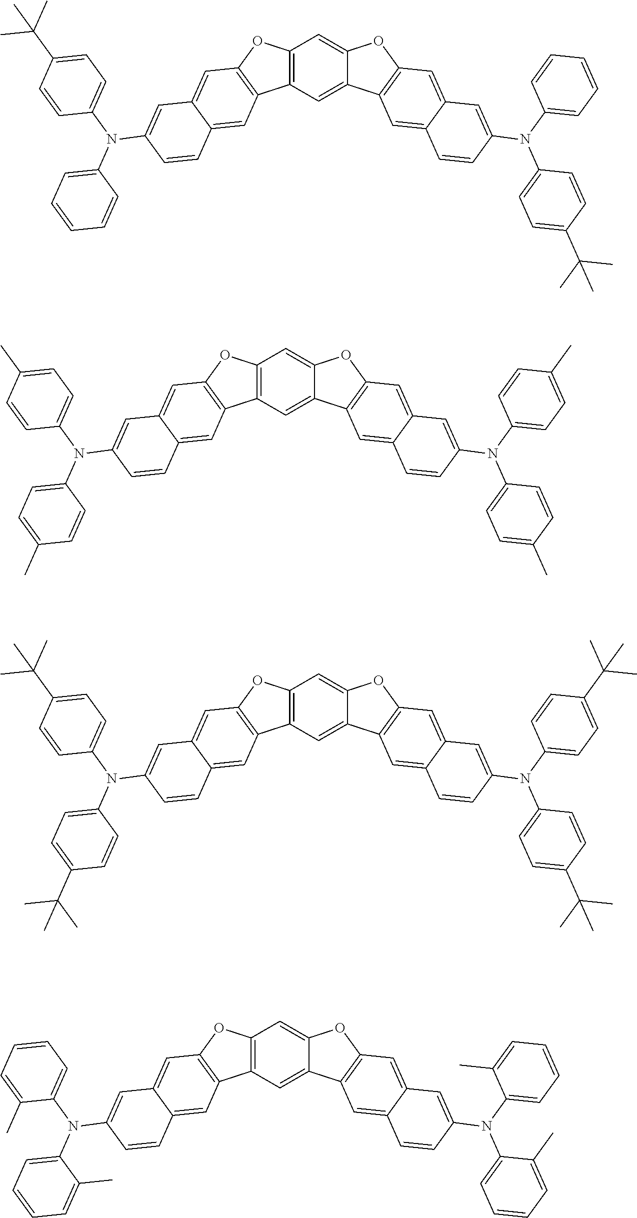

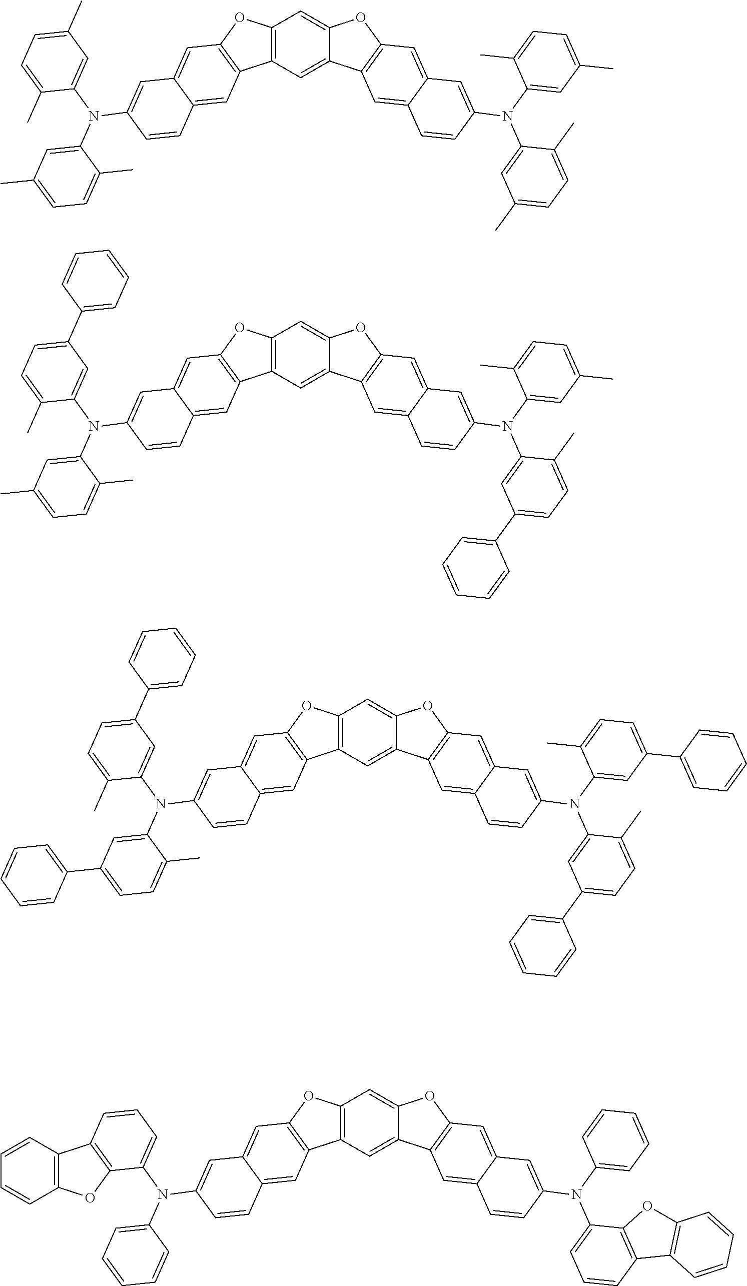

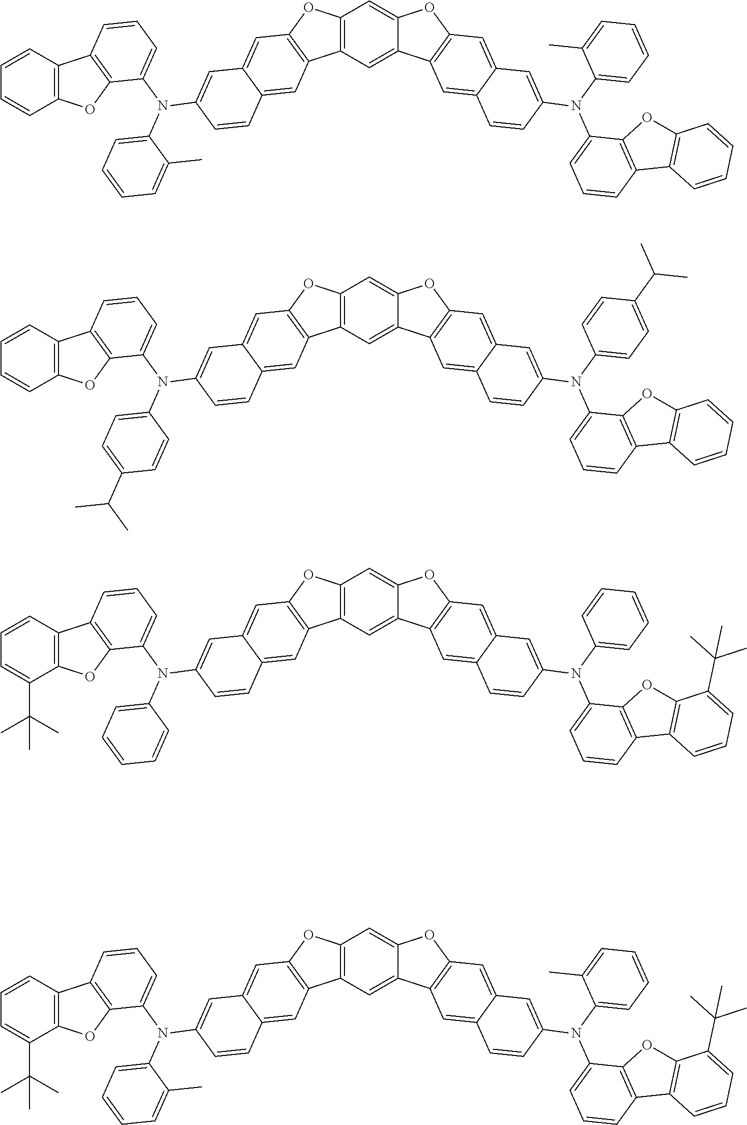 [SMILES: Cc1ccccc1N(c1ccc2cc3c(cc2c1)oc1cc2oc4cc5cc(N(c6ccccc6C)c6cccc7c6oc6ccccc67)ccc5cc4c2cc13)c1cccc2c1oc1ccccc12 ; CC(C)c1ccc(N(c2ccc3cc4c(cc3c2)oc2cc3oc5cc6cc(N(c7ccc(C(C)C)cc7)c7cccc8c7oc7ccccc78)ccc6cc5c3cc24)c2cccc3c2oc2ccccc23)cc1 ; CC(C)(C)c1cccc2c1oc1c(N(c3ccccc3)c3ccc4cc5c(cc4c3)oc3cc4oc6cc7cc(N(c8ccccc8)c8cccc9c8oc8c(C(C)(C)C)cccc89)ccc7cc6c4cc35)cccc12 ; Cc1ccccc1N(c1ccc2cc3c(cc2c1)oc1cc2oc4cc5cc(N(c6ccccc6C)c6cccc7c6oc6c(C(C)(C)C)cccc67)ccc5cc4c2cc13)c1cccc2c1oc1c(C(C)(C)C)cccc12]

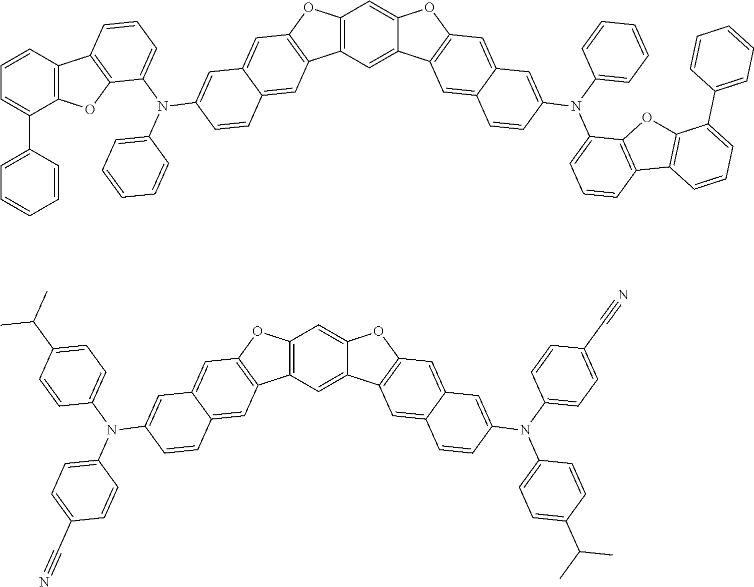 [SMILES: c1ccc(-c2cccc3c2oc2c(N(c4ccccc4)c4ccc5cc6c(cc5c4)oc4cc5oc7cc8cc(N(c9ccccc9)c9cccc%10c9oc9c(-c%11ccccc%11)cccc9%10)ccc8cc7c5cc46)cccc23)cc1 ; CC(C)c1ccc(N(c2ccc(C#N)cc2)c2ccc3cc4c(cc3c2)oc2cc3oc5cc6cc(N(c7ccc(C#N)cc7)c7ccc(C(C)C)cc7)ccc6cc5c3cc24)cc1]

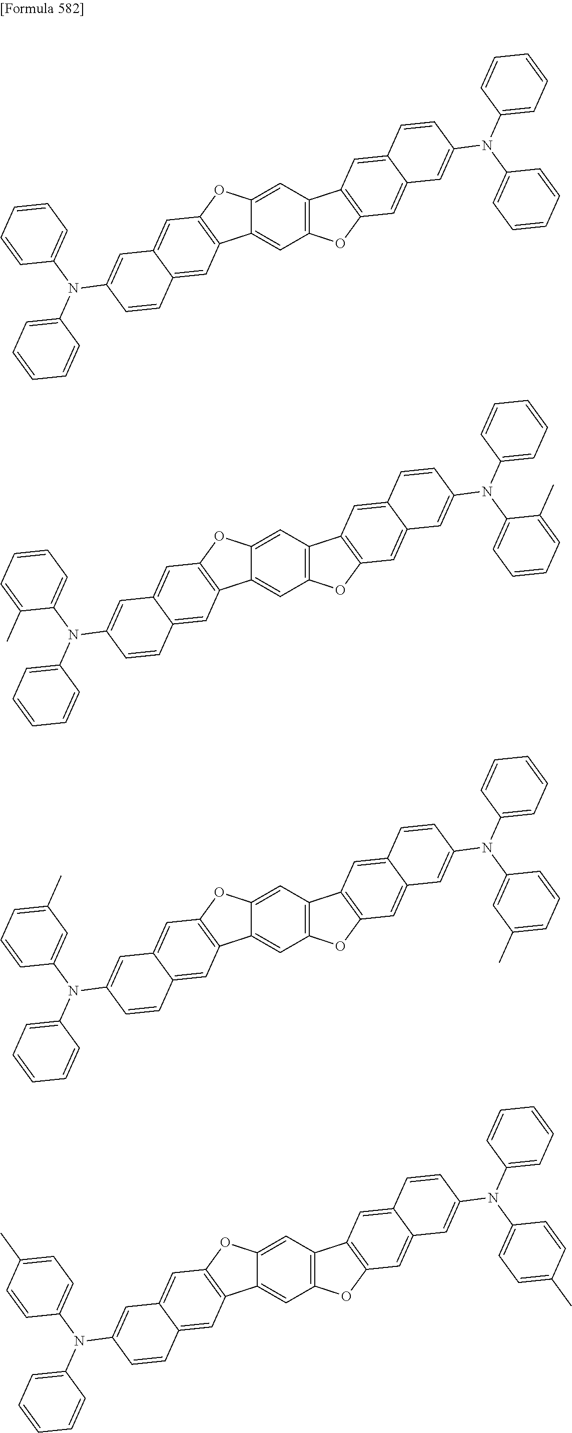

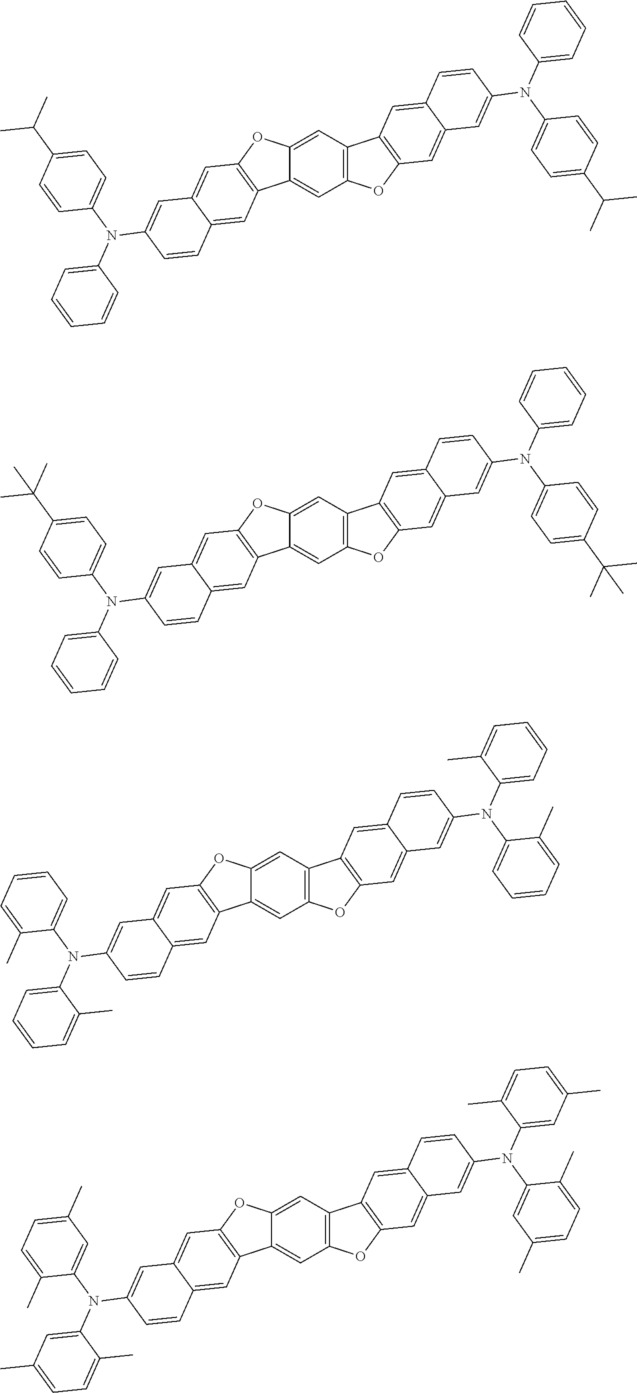

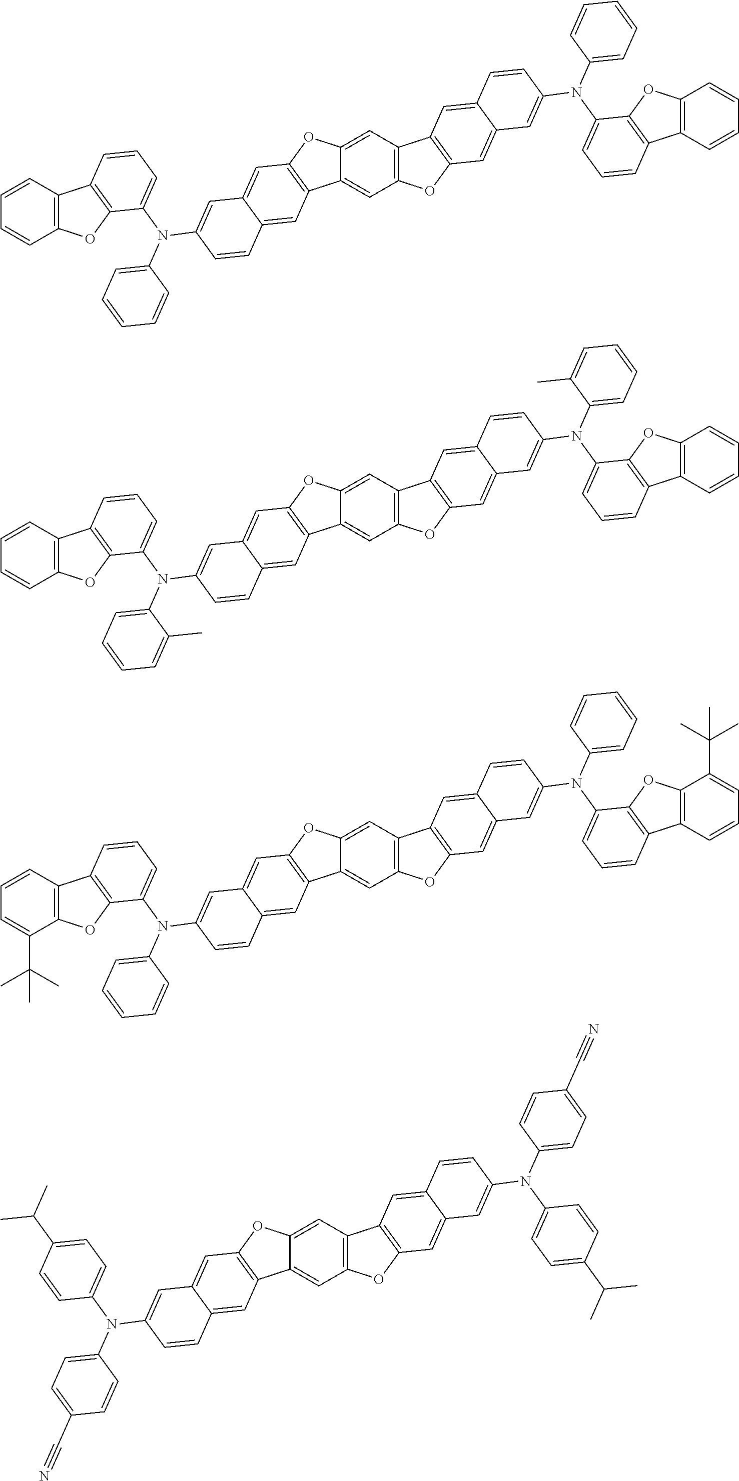

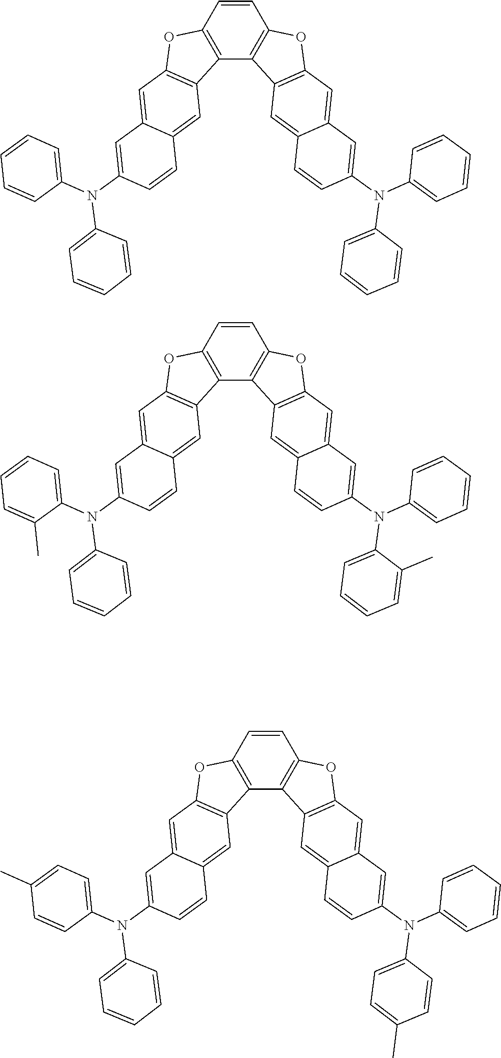 [SMILES: c1ccc(N(c2ccccc2)c2ccc3cc4c(cc3c2)oc2ccc3oc5cc6cc(N(c7ccccc7)c7ccccc7)ccc6cc5c3c24)cc1 ; Cc1ccccc1N(c1ccccc1)c1ccc2cc3c(cc2c1)oc1ccc2oc4cc5cc(N(c6ccccc6)c6ccccc6C)ccc5cc4c2c13 ; Cc1ccc(N(c2ccccc2)c2ccc3cc4c(cc3c2)oc2ccc3oc5cc6cc(N(c7ccccc7)c7ccc(C)cc7)ccc6cc5c3c24)cc1]

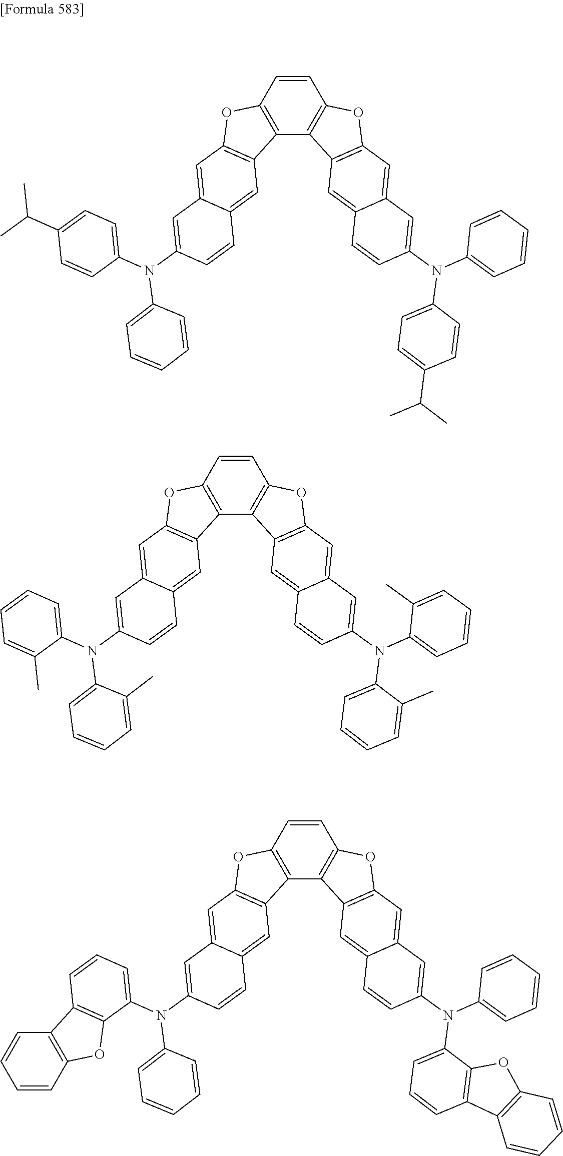 [SMILES: CC(C)c1ccc(N(c2ccccc2)c2ccc3cc4c(cc3c2)oc2ccc3oc5cc6cc(N(c7ccccc7)c7ccc(C(C)C)cc7)ccc6cc5c3c24)cc1 ; Cc1ccccc1N(c1ccc2cc3c(cc2c1)oc1ccc2oc4cc5cc(N(c6ccccc6C)c6ccccc6C)ccc5cc4c2c13)c1ccccc1C ; c1ccc(N(c2ccc3cc4c(cc3c2)oc2ccc3oc5cc6cc(N(c7ccccc7)c7cccc8c7oc7ccccc78)ccc6cc5c3c24)c2cccc3c2oc2ccccc23)cc1]

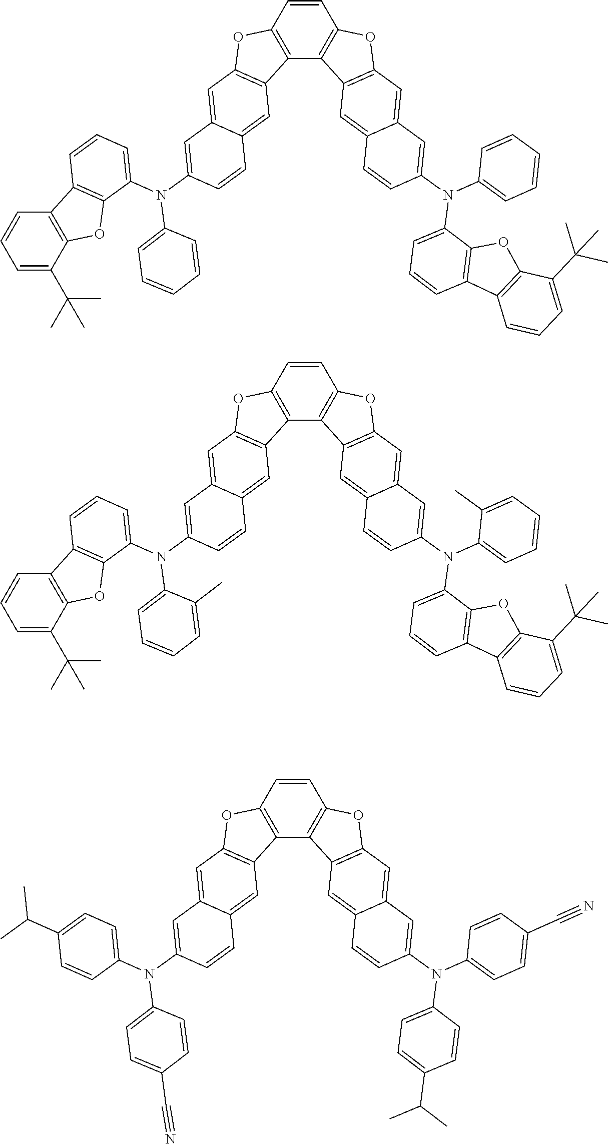

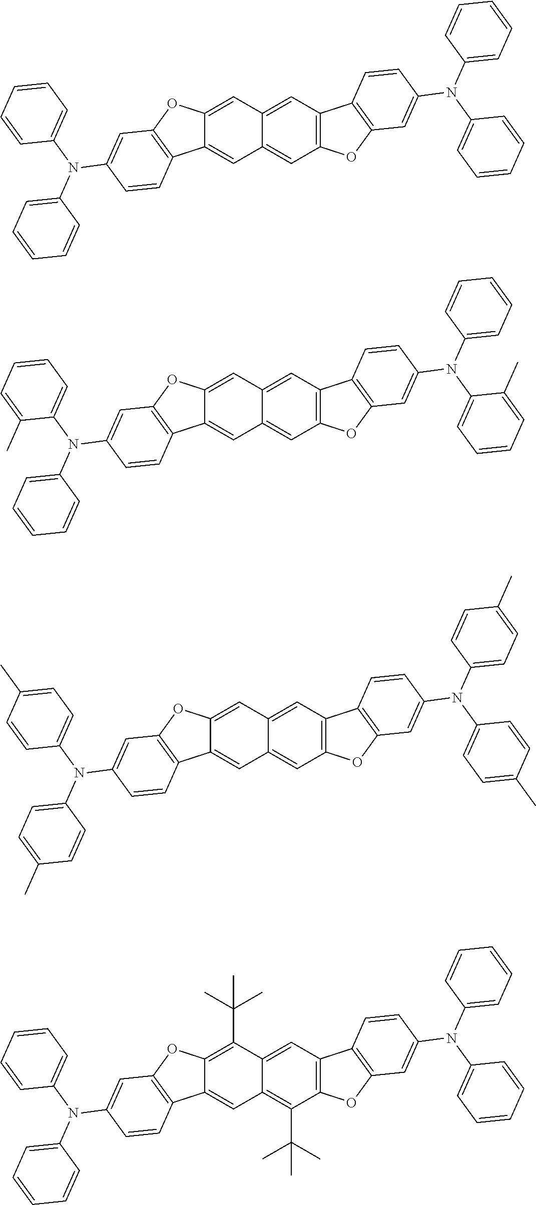

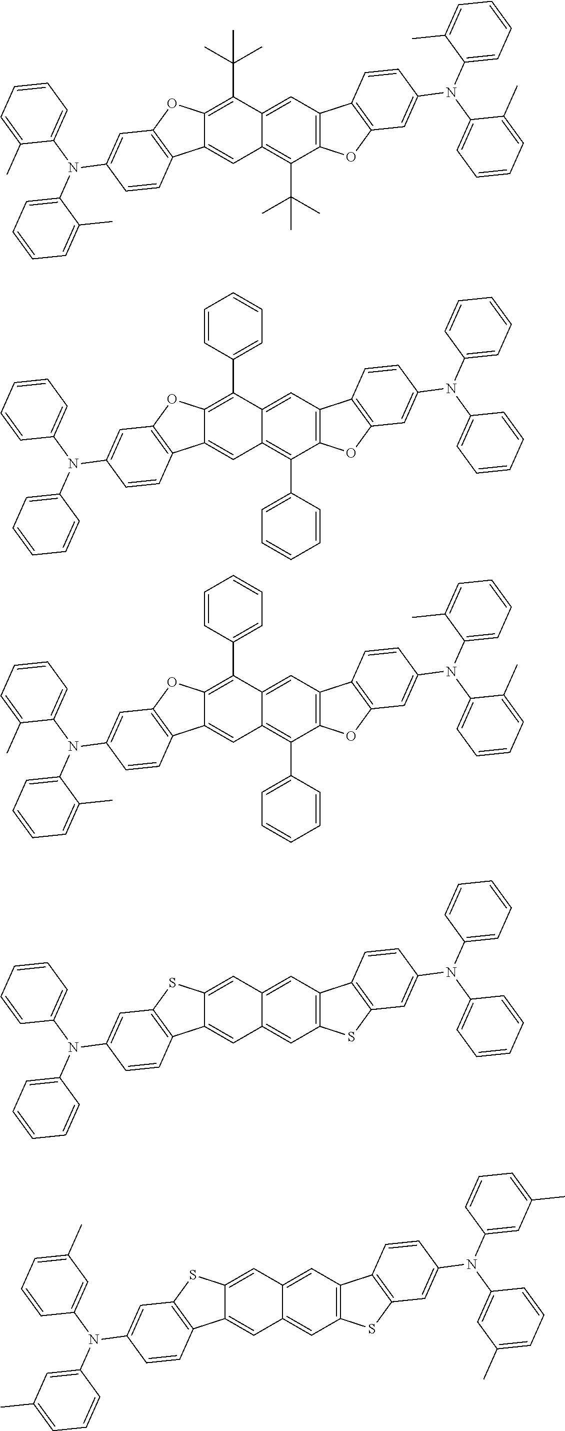

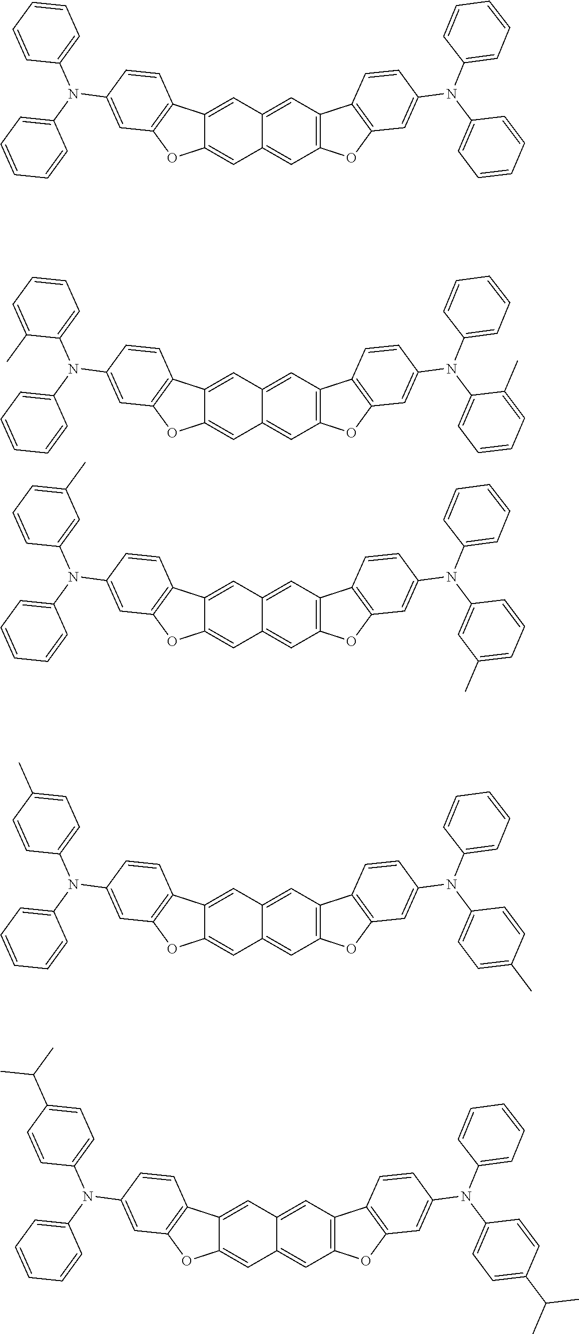

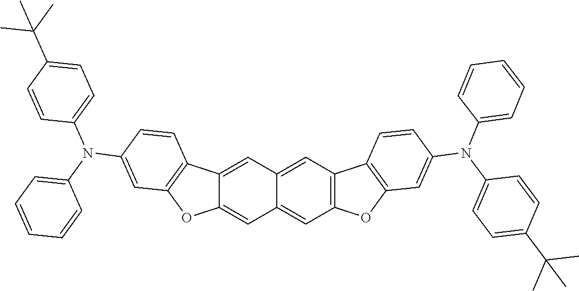 [SMILES: CC(C)(C)c1ccc(N(c2ccccc2)c2ccc3c(c2)oc2cc4cc5oc6cc(N(c7ccccc7)c7ccc(C(C)(C)C)cc7)ccc6c5cc4cc23)cc1]

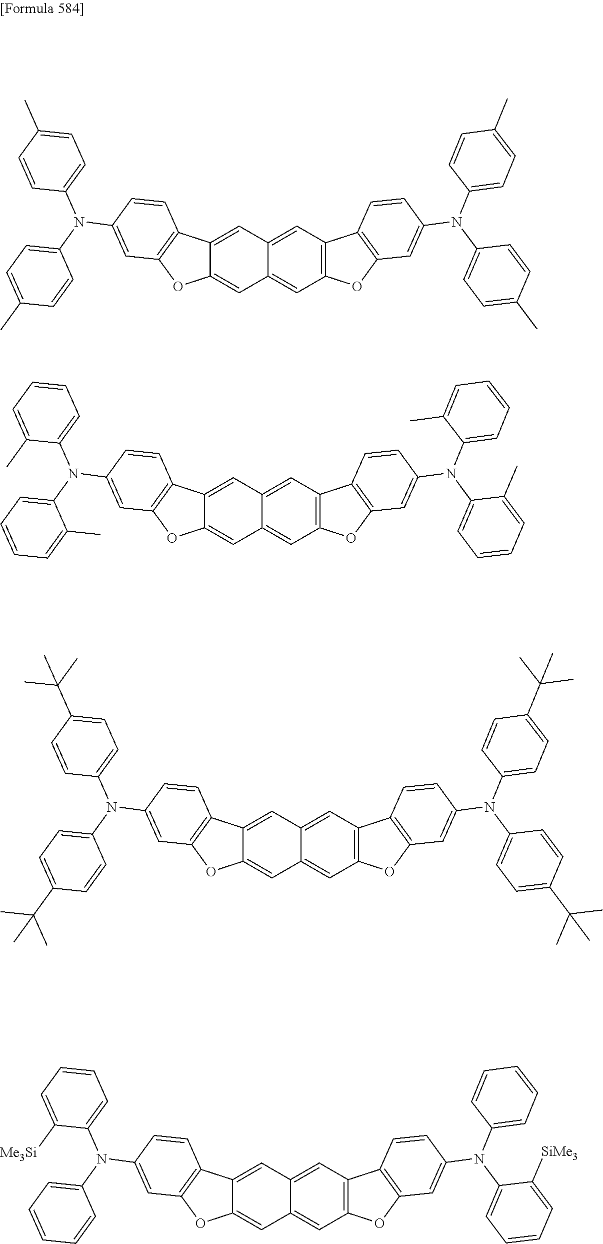

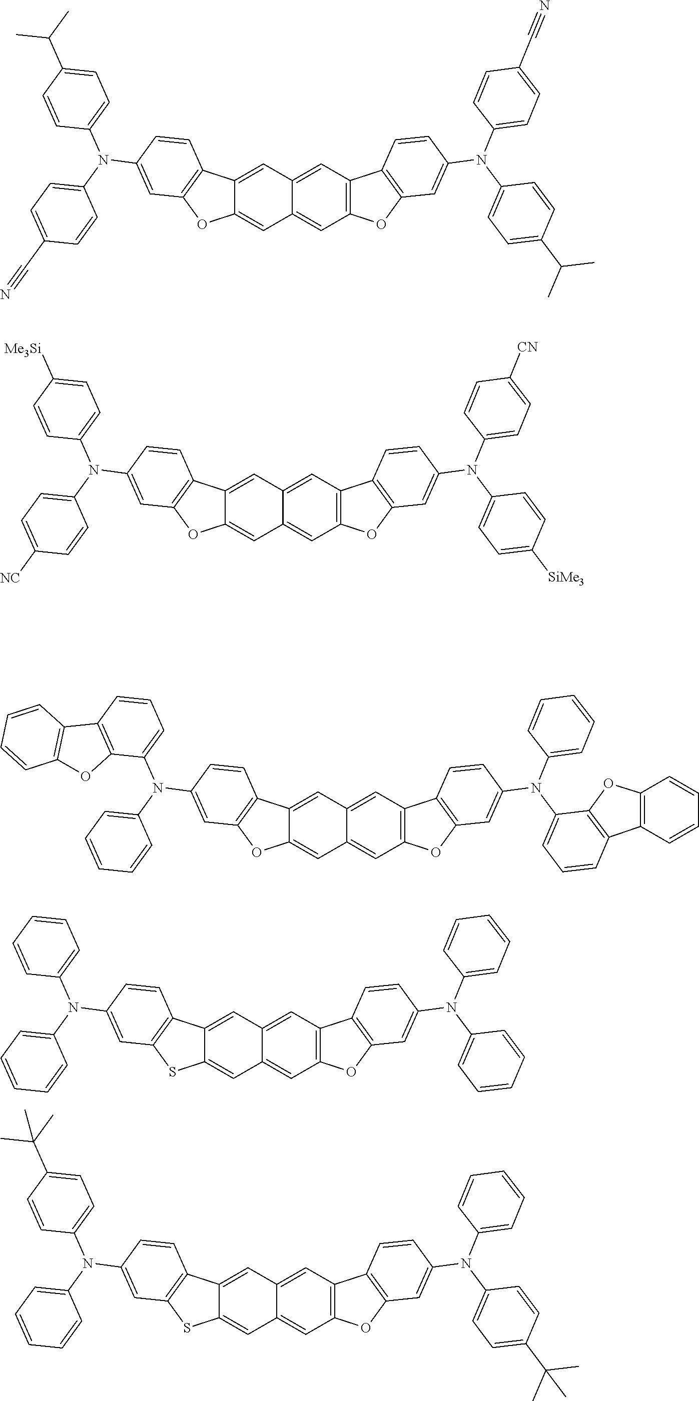

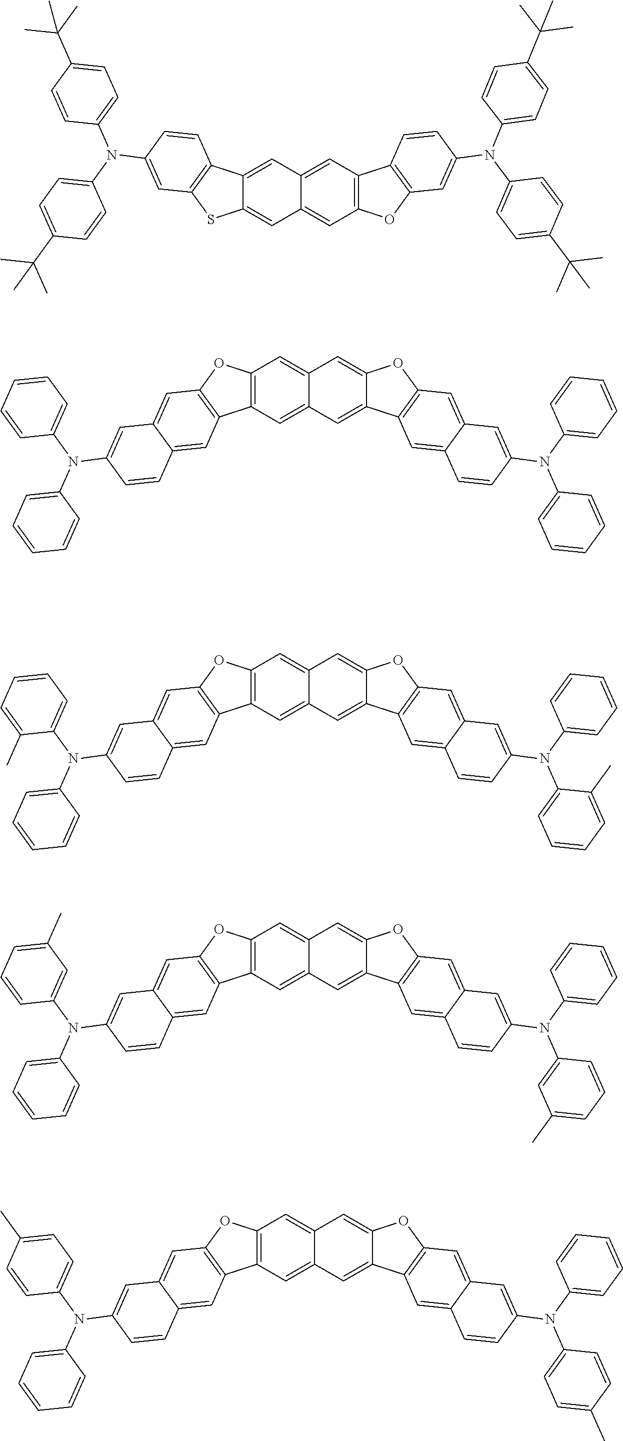

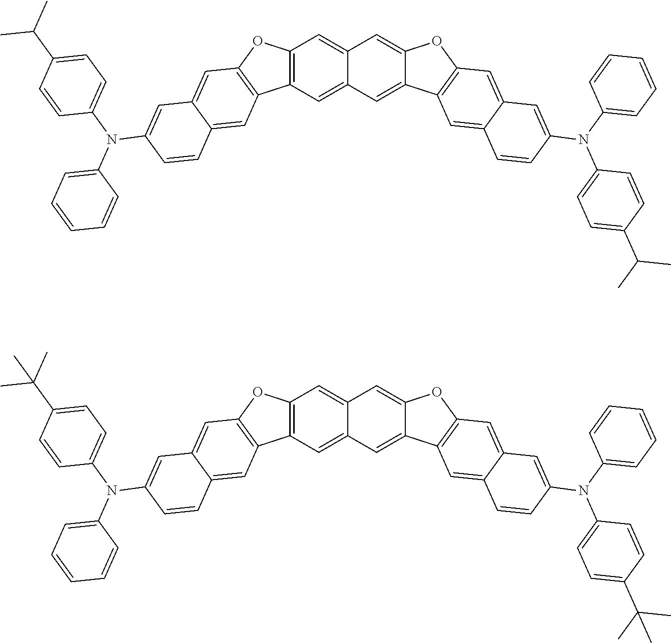

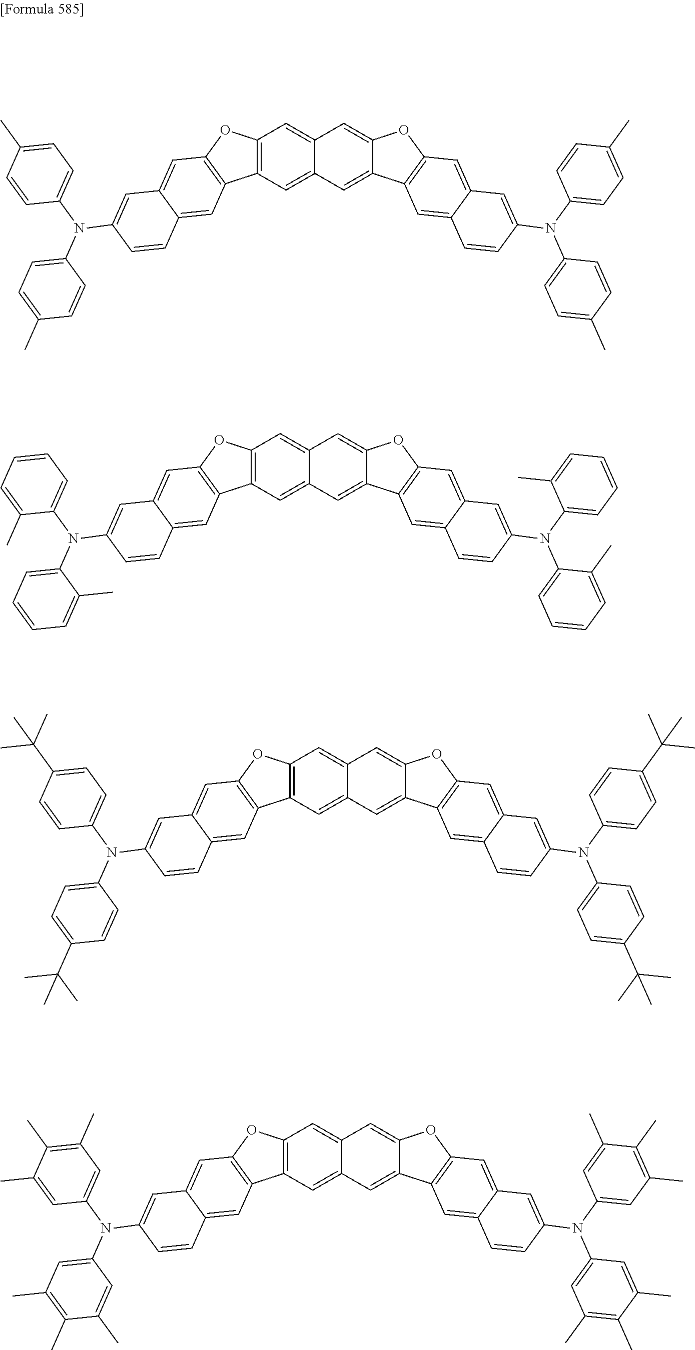

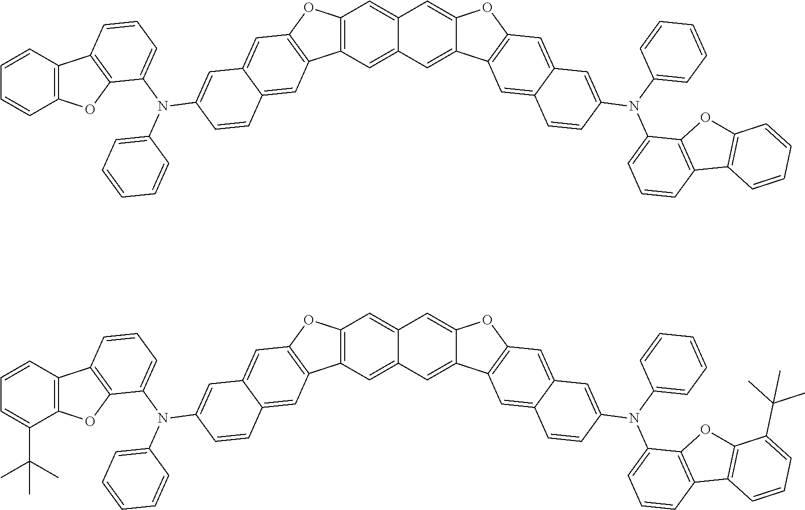 [SMILES: c1ccc(N(c2ccc3cc4c(cc3c2)oc2cc3cc5oc6cc7cc(N(c8ccccc8)c8cccc9c8oc8ccccc89)ccc7cc6c5cc3cc24)c2cccc3c2oc2ccccc23)cc1 ; CC(C)(C)c1cccc2c1oc1c(N(c3ccccc3)c3ccc4cc5c(cc4c3)oc3cc4cc6oc7cc8cc(N(c9ccccc9)c9cccc%10c9oc9c(C(C)(C)C)cccc9%10)ccc8cc7c6cc4cc35)cccc12]

### Compound Represented by Formula (8)

The compound represented by the formula (8) will be described below.

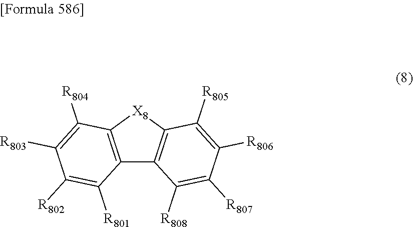

In the formula (8):
at least one combination of R₈₀₁ and R₈₀₂, R₈₀₂ and R₈₀₃, or R₈₀₃ and R₈₀₄ are mutually bonded to form a divalent group represented by a formula (82) below; and
at least one combination of R₈₀₅ and R₈₀₆, R₈₀₆ and R₈₀₇, or R₈₀₇ and R₈₀₈ are mutually bonded to form a divalent group represented by a formula (83) below.

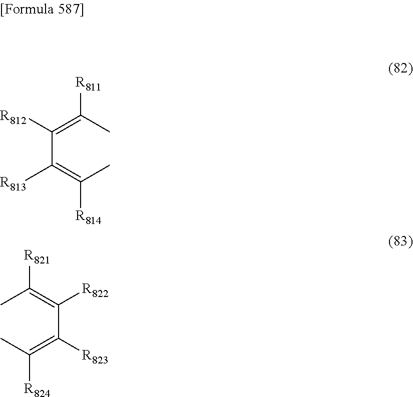

At least one of R₈₀₁ to R₈₀₄ not forming the divalent group represented by the formula (82) or R₈₁₁ to R₈₁₄ is a monovalent group represented by a formula (84) below;
at least one of R₈₀₅ to R₈₀₈ not forming the divalent group represented by the formula (83) or R₈₂₁ to R₈₂₄ is a monovalent group represented by a formula (84) below;
X₈ is an oxygen atom, a sulfur atom, or NR₈₀₉; and
R₈₀₁ to R₈₀₈ not forming the divalent group represented by the formula (82) or (83) and not being the monovalent group represented by the formula (84), R₈₁₁ to R₈₁₄ and R₈₂₁ to R₈₂₄ not being the monovalent group represented by the formula (84), and R₈₀₉ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

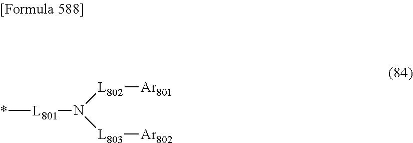

In the formula (84):
Ar₈₀₁ and Ar₈₀₂ are each independently a substituted or unsubstituted aryl group having 6 to 50 ring carbon atoms, or a substituted or unsubstituted heterocyclic group having 5 to 50 ring atoms;
L₈₀₁ to L₈₀₃ are each independently a single bond, a substituted or unsubstituted arylene group having 6 to 30 ring carbon atoms, a substituted or unsubstituted divalent heterocyclic group having 5 to 30 ring atoms, or a divalent linking group formed by bonding two, three or four groups selected from the group consisting of a substituted or unsubstituted arylene group having 6 to 30 ring carbon atoms and a substituted or unsubstituted divalent heterocyclic group having 5 to 30 ring atoms; and
* in the formula (84) represents a bonding position to a cyclic structure represented by the formula (8) or a bonding position to a group represented by the formula (82) or (83).

In the formula (8), the positions for the divalent group represented by the formula (82) and the divalent group represented by the formula (83) to be formed are not specifically limited but the divalent groups may be formed at any possible positions on R₈₀₁ to R₈₀₈.

In an exemplary embodiment, the compound represented by the formula (8) is represented by any one of formulae (81-1) to (81-6) below.

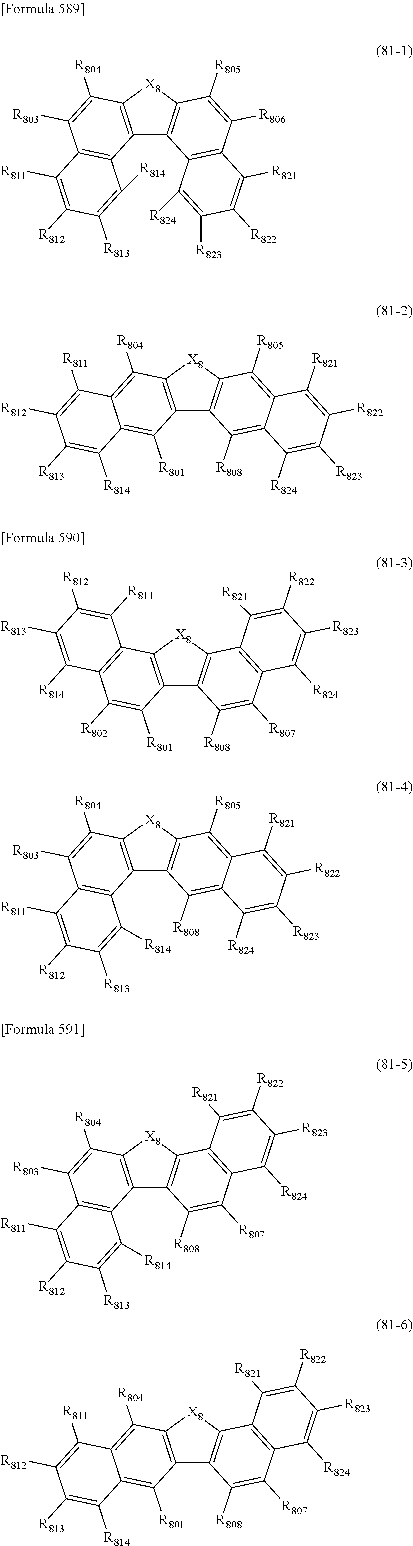

In the formulae (81-1) to (81-6):
X₈ represents the same as X₈ in the formula (8);
at least two of R₈₀₁ to R₈₂₄ are each a monovalent group represented by the formula (84); and
R₈₀₁ to R₈₂₄ not being the monovalent group represented by the formula (84) are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, the compound represented by the formula (8) is represented by any one of formulae (81-7) to (81-18) below.

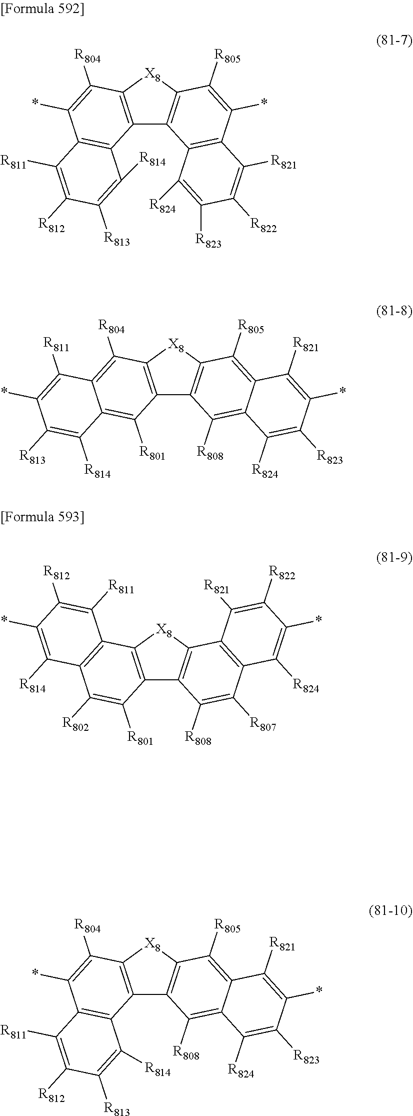

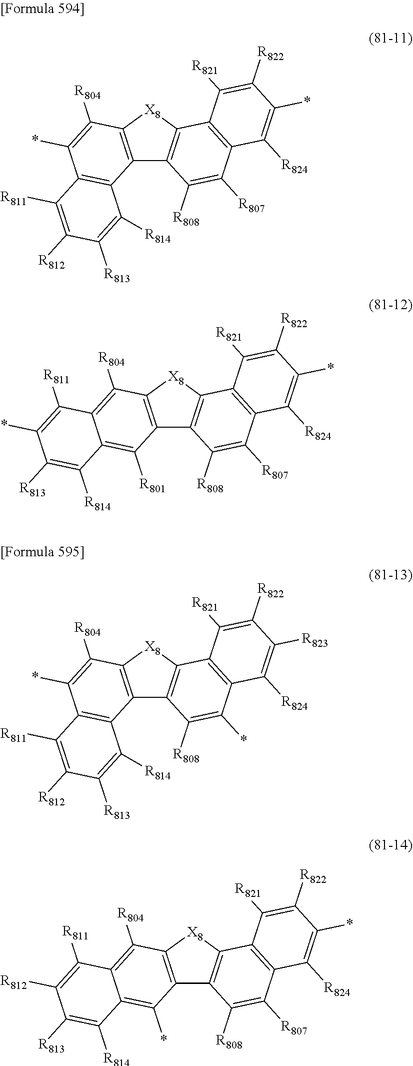

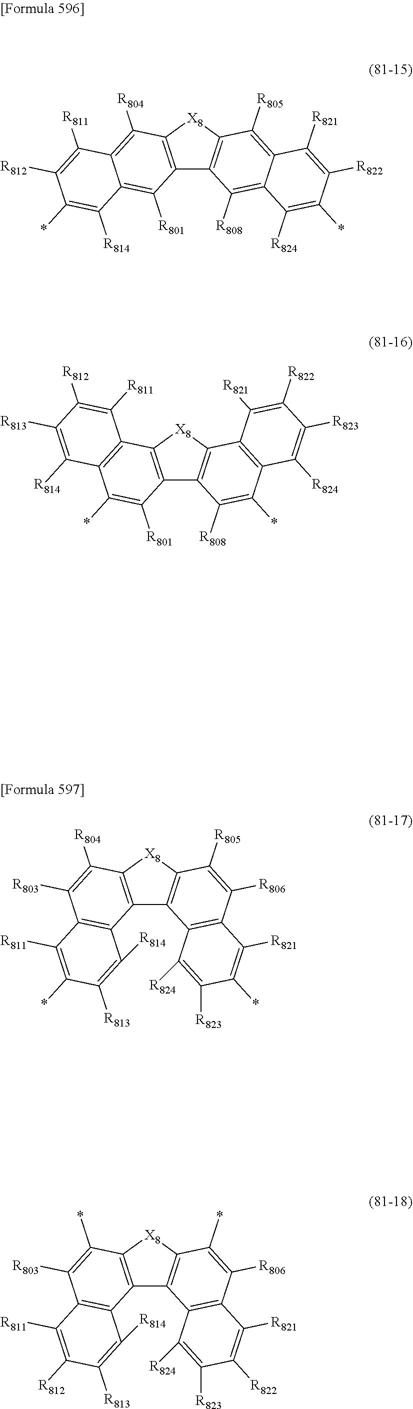

In the formulae (81-7) to (81-18):
X₈ represents the same as X₈ in the formula (8);
* is a single bond bonded to a monovalent group represented by the formula (84); and
R₈₀₁ to R₈₂₄ each independently represent the same as R₈₀₁ to R₈₂₄ in the formulae (81-1) to (81-6) that are not the monovalent group represented by the formula (84).

R₈₀₁ to R₈os not forming the divalent group represented by the formula (82) or (83) and not being the monovalent group represented by the formula (84), and R₈₁₁ to R₈₁₄ and R₈₂₁ to R₈₂₄ not being the monovalent group represented by the formula (84) are preferably each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

The monovalent group represented by the formula (84) is preferably represented by a formula (85) or (86) below.

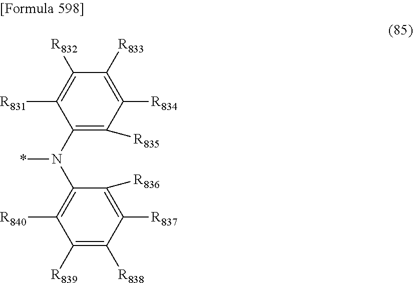

In the formula (85):
R₈₃₁ to R₈₄₀ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
* in the formula (85) represents the same as * in the formula (84).

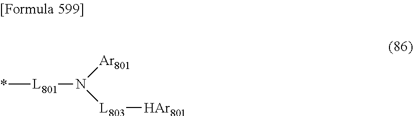

In the formula (86):
Ar₈₀₁, L₈₀₁, and L₈₀₃ represent the same as Ar₁₀₁, L₈₀₁, and L₈₀₃ in the formula (84); and
HAr₈₀₁ is a structure represented by a formula (87) below.

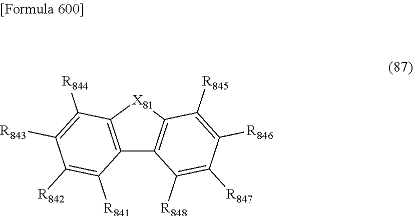

In the formula (87):
X₈₁ is an oxygen atom or a sulfur atom;
one of R₈₄₁ to R₈₄₈ is a single bond with L₈₀₃; and
R₈₄₁ to R₈₄₈ not being the single bond are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

Specific examples of the compound represented by the formula (8) include compounds shown below as well as the compounds disclosed in WO 2014/104144.

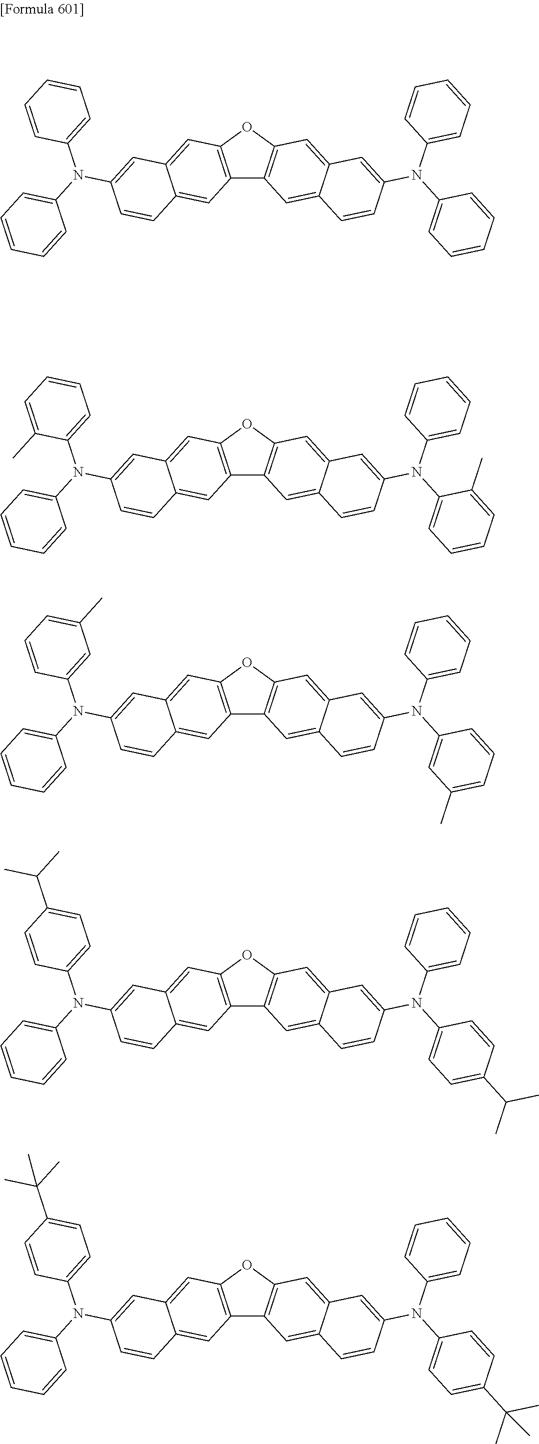 [SMILES: c1ccc(N(c2ccccc2)c2ccc3cc4c(cc3c2)oc2cc3cc(N(c5ccccc5)c5ccccc5)ccc3cc24)cc1 ; Cc1ccccc1N(c1ccccc1)c1ccc2cc3c(cc2c1)oc1cc2cc(N(c4ccccc4)c4ccccc4C)ccc2cc13 ; Cc1cccc(N(c2ccccc2)c2ccc3cc4c(cc3c2)oc2cc3cc(N(c5ccccc5)c5cccc(C)c5)ccc3cc24)c1 ; CC(C)c1ccc(N(c2ccccc2)c2ccc3cc4c(cc3c2)oc2cc3cc(N(c5ccccc5)c5ccc(C(C)C)cc5)ccc3cc24)cc1 ; CC(C)(C)c1ccc(N(c2ccccc2)c2ccc3cc4c(cc3c2)oc2cc3cc(N(c5ccccc5)c5ccc(C(C)(C)C)cc5)ccc3cc24)cc1]

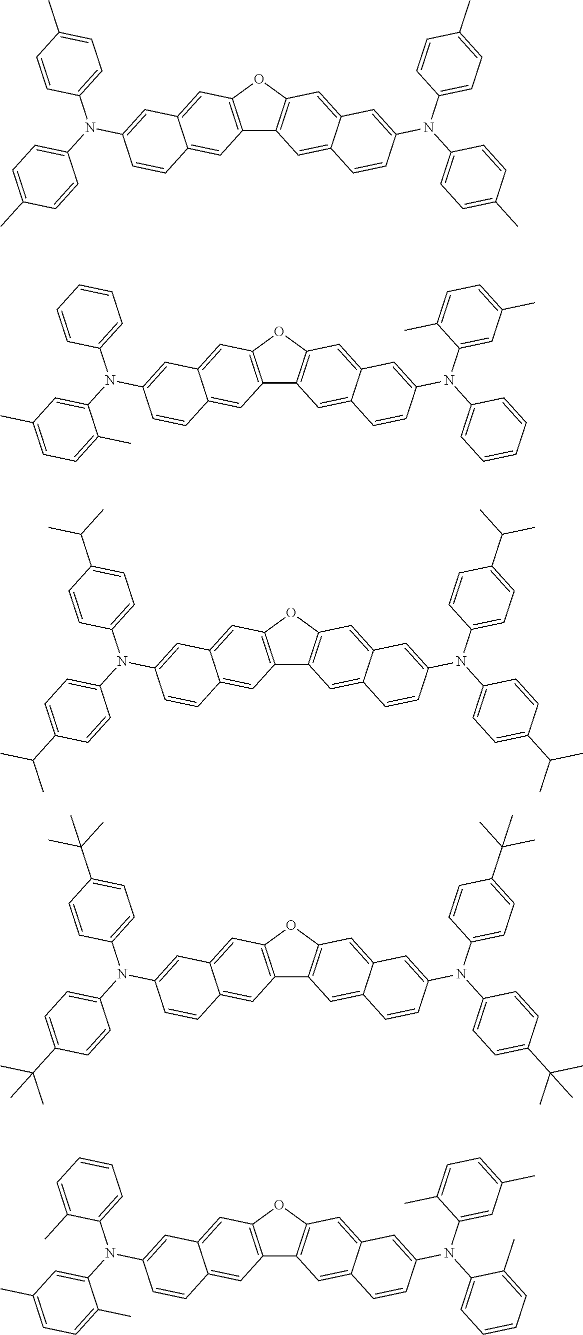

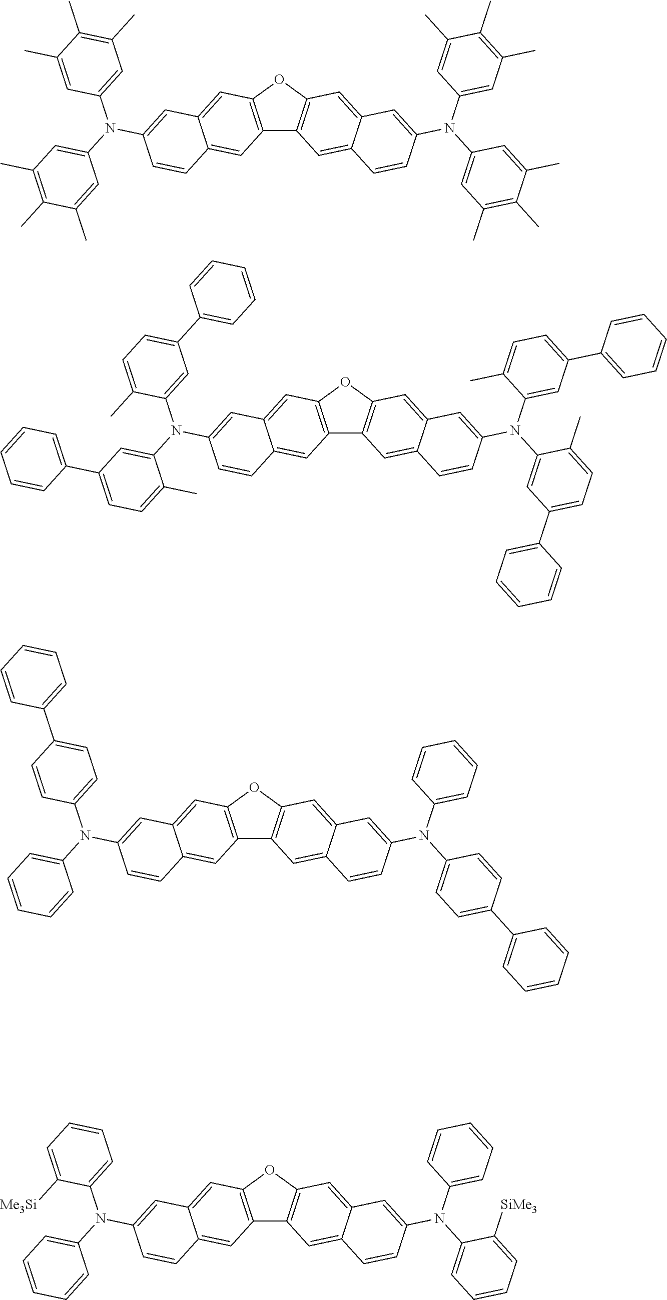

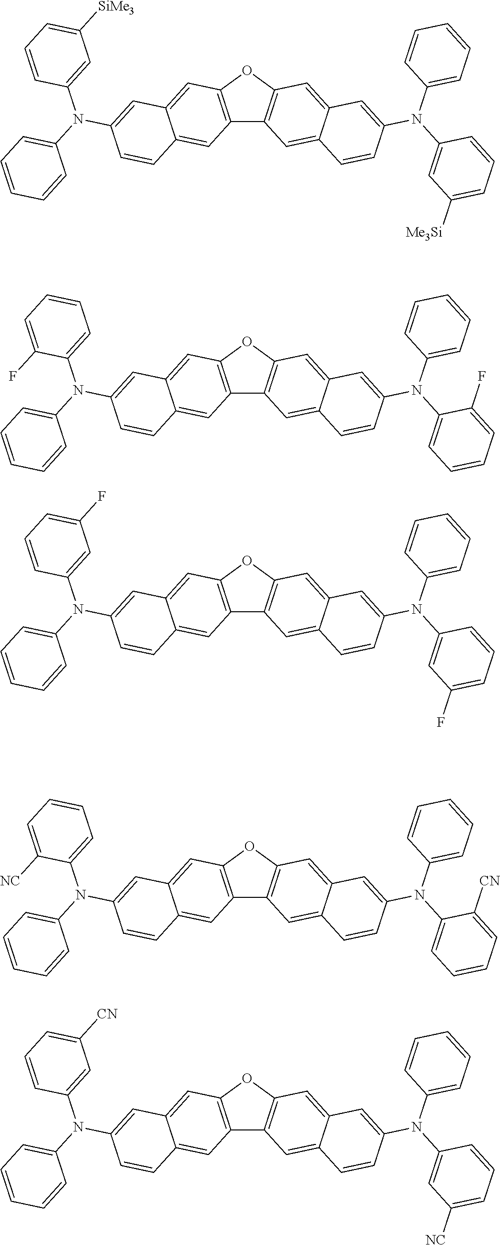

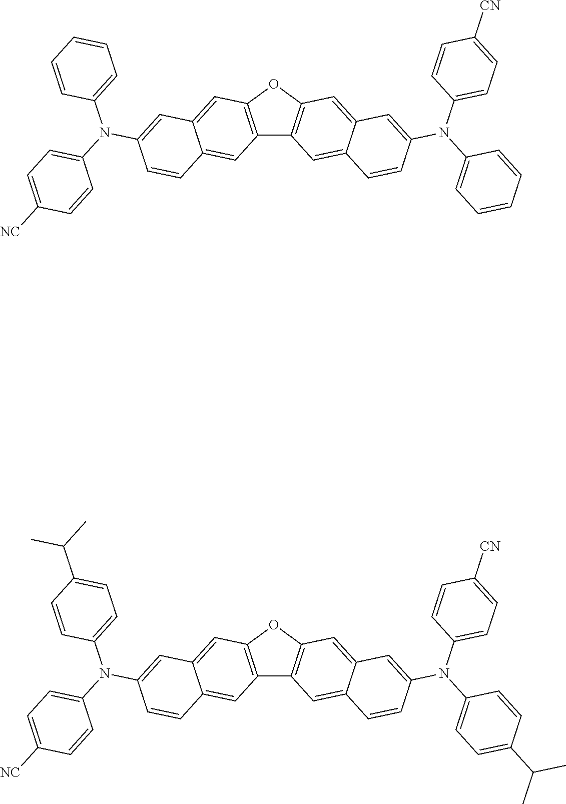 [SMILES: N#Cc1ccc(N(c2ccccc2)c2ccc3cc4c(cc3c2)oc2cc3cc(N(c5ccccc5)c5ccc(C#N)cc5)ccc3cc24)cc1 ; CC(C)c1ccc(N(c2ccc(C#N)cc2)c2ccc3cc4c(cc3c2)oc2cc3cc(N(c5ccc(C#N)cc5)c5ccc(C(C)C)cc5)ccc3cc24)cc1]

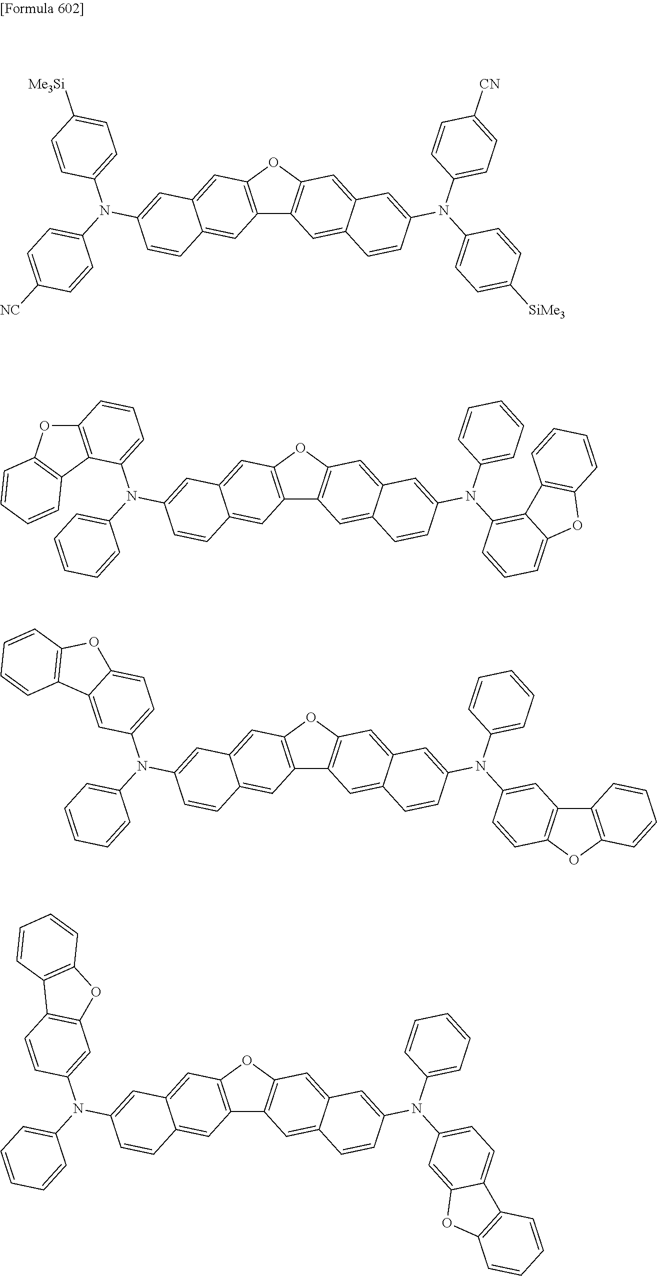 [SMILES: C[Si](C)(C)c1ccc(N(c2ccc(C#N)cc2)c2ccc3cc4c(cc3c2)oc2cc3cc(N(c5ccc(C#N)cc5)c5ccc([Si](C)(C)C)cc5)ccc3cc24)cc1 ; c1ccc(N(c2ccc3cc4c(cc3c2)oc2cc3cc(N(c5ccccc5)c5cccc6oc7ccccc7c56)ccc3cc24)c2cccc3oc4ccccc4c23)cc1 ; c1ccc(N(c2ccc3cc4c(cc3c2)oc2cc3cc(N(c5ccccc5)c5ccc6oc7ccccc7c6c5)ccc3cc24)c2ccc3oc4ccccc4c3c2)cc1 ; c1ccc(N(c2ccc3cc4c(cc3c2)oc2cc3cc(N(c5ccccc5)c5ccc6c(c5)oc5ccccc56)ccc3cc24)c2ccc3c(c2)oc2ccccc23)cc1]

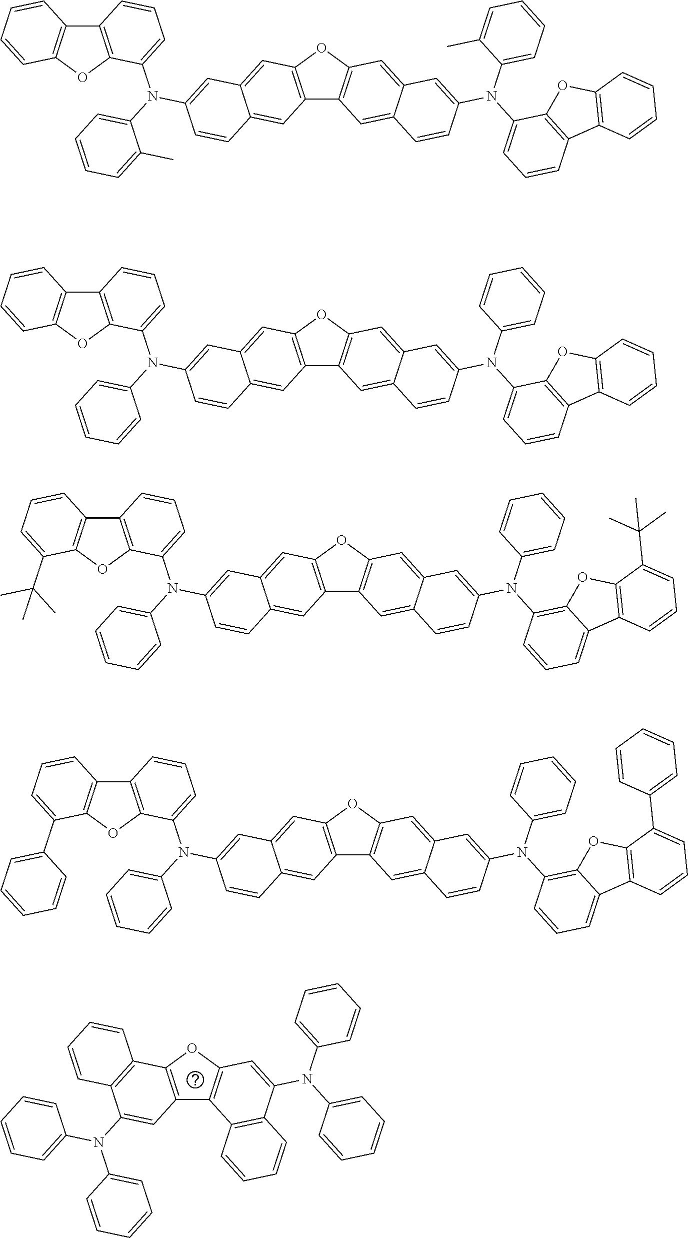

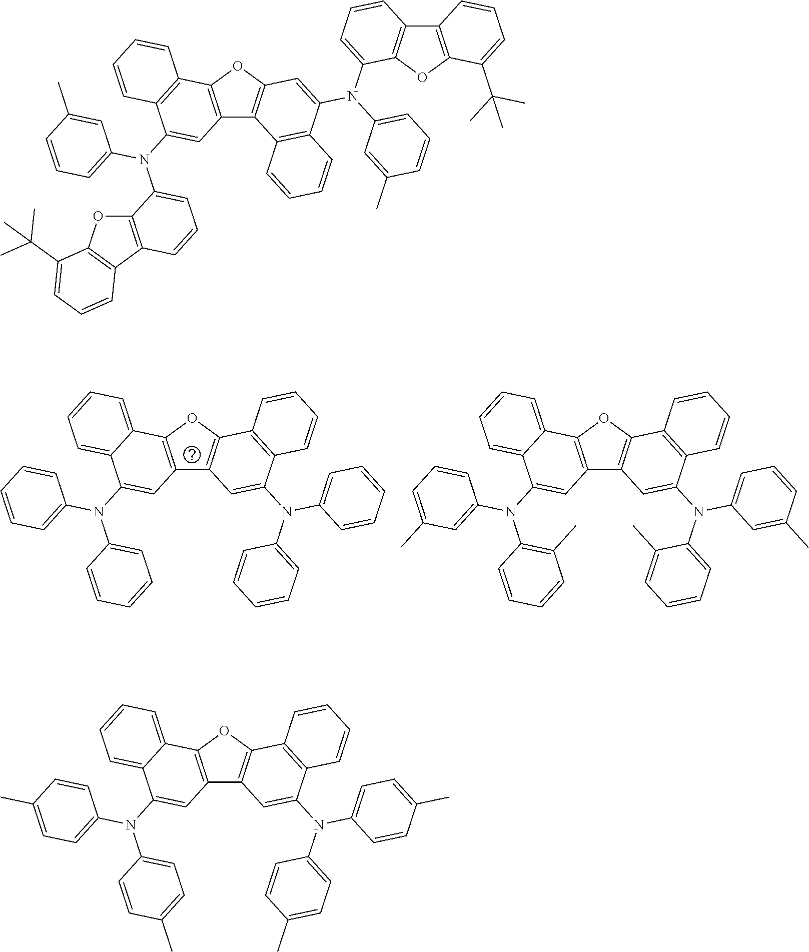

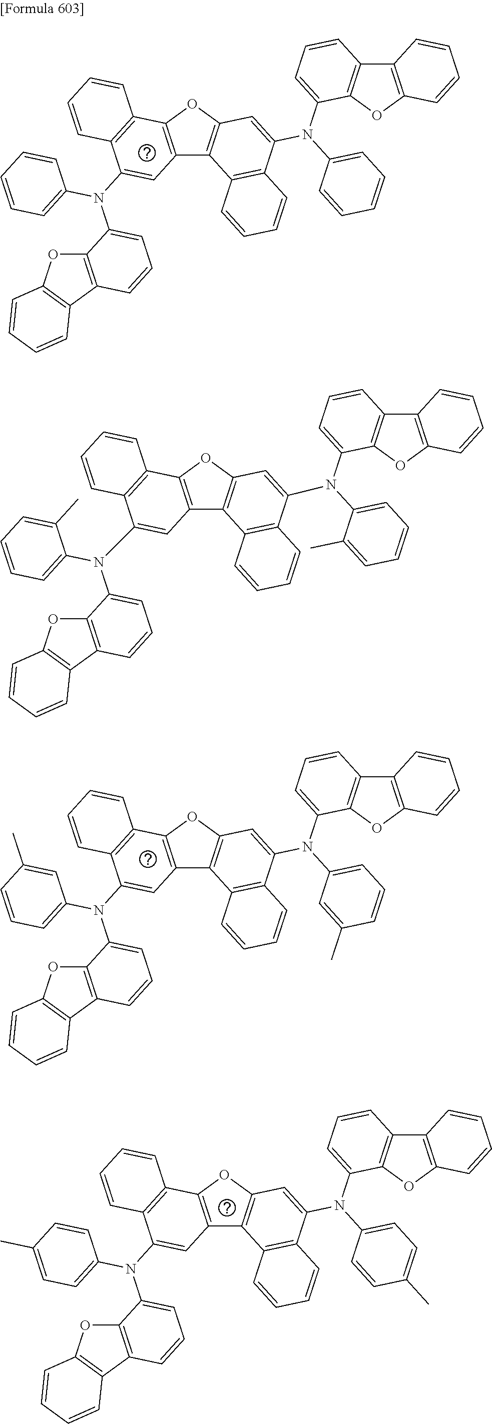 [SMILES: c1ccc(N(c2cc3c(oc4cc(N(c5ccccc5)c5cccc6c5oc5ccccc56)c5ccccc5c43)c3ccccc23)c2cccc3c2oc2ccccc23)cc1 ; Cc1ccccc1N(c1cc2c(oc3cc(N(c4ccccc4C)c4cccc5c4oc4ccccc45)c4ccccc4c32)c2ccccc12)c1cccc2c1oc1ccccc12 ; Cc1cccc(N(c2cc3c(oc4cc(N(c5cccc(C)c5)c5cccc6c5oc5ccccc56)c5ccccc5c43)c3ccccc23)c2cccc3c2oc2ccccc23)c1 ; Cc1ccc(N(c2cc3c(oc4cc(N(c5ccc(C)cc5)c5cccc6c5oc5ccccc56)c5ccccc5c43)c3ccccc23)c2cccc3c2oc2ccccc23)cc1]

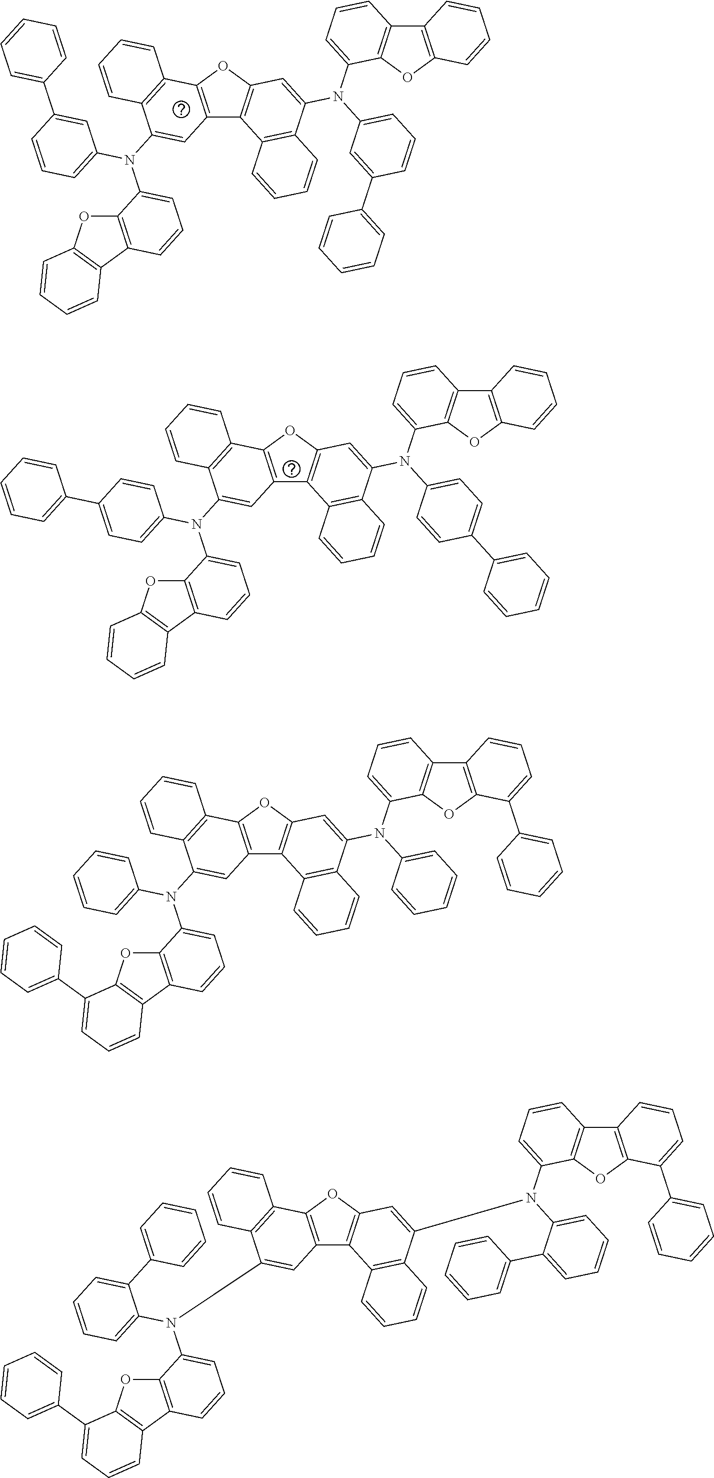

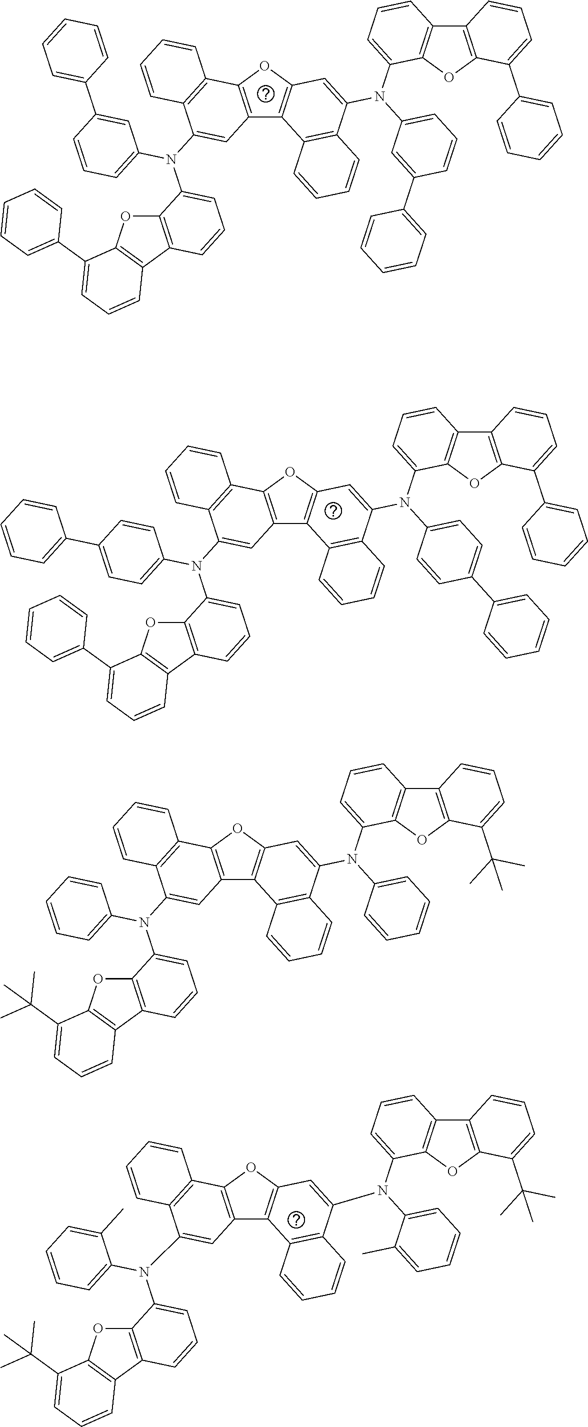

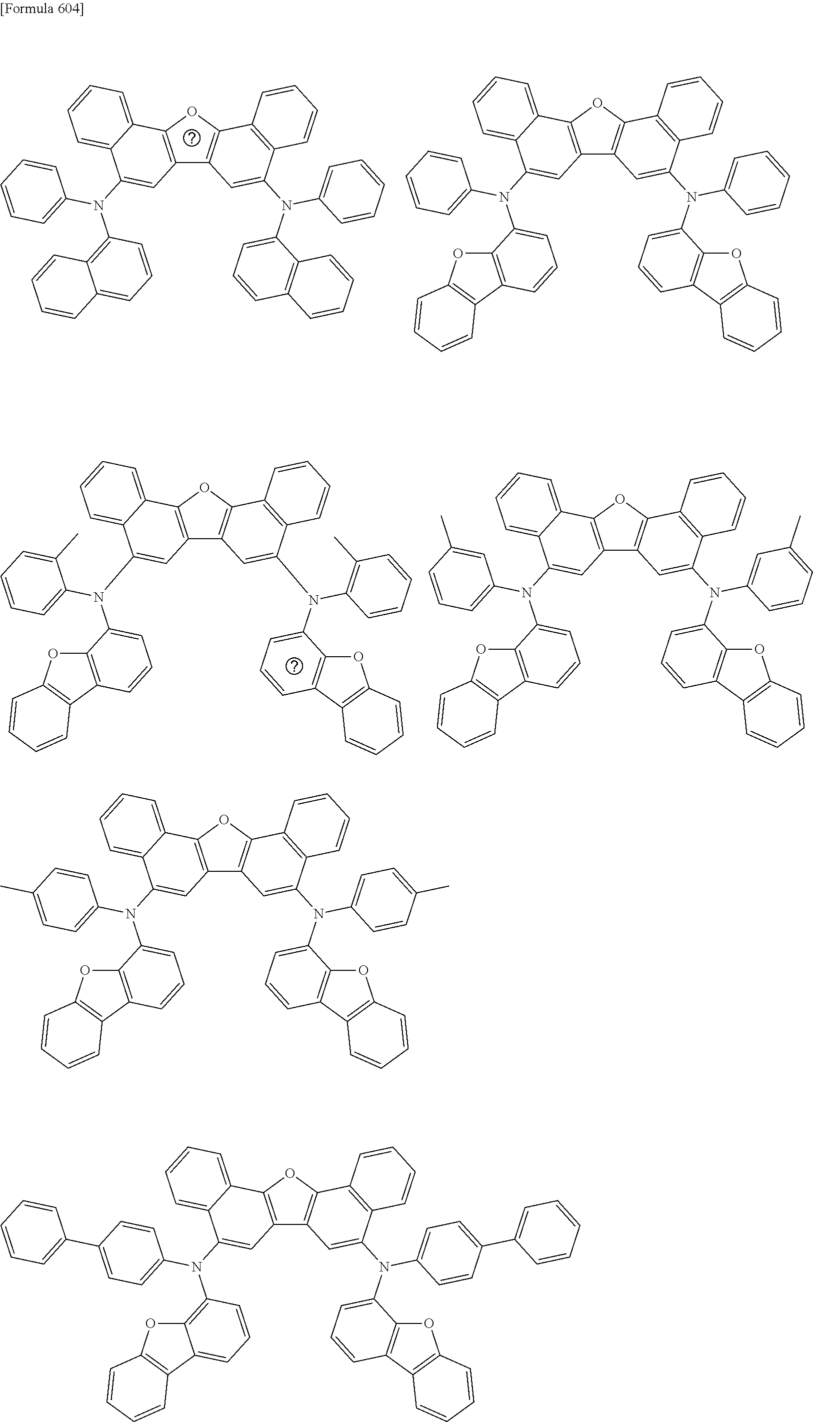

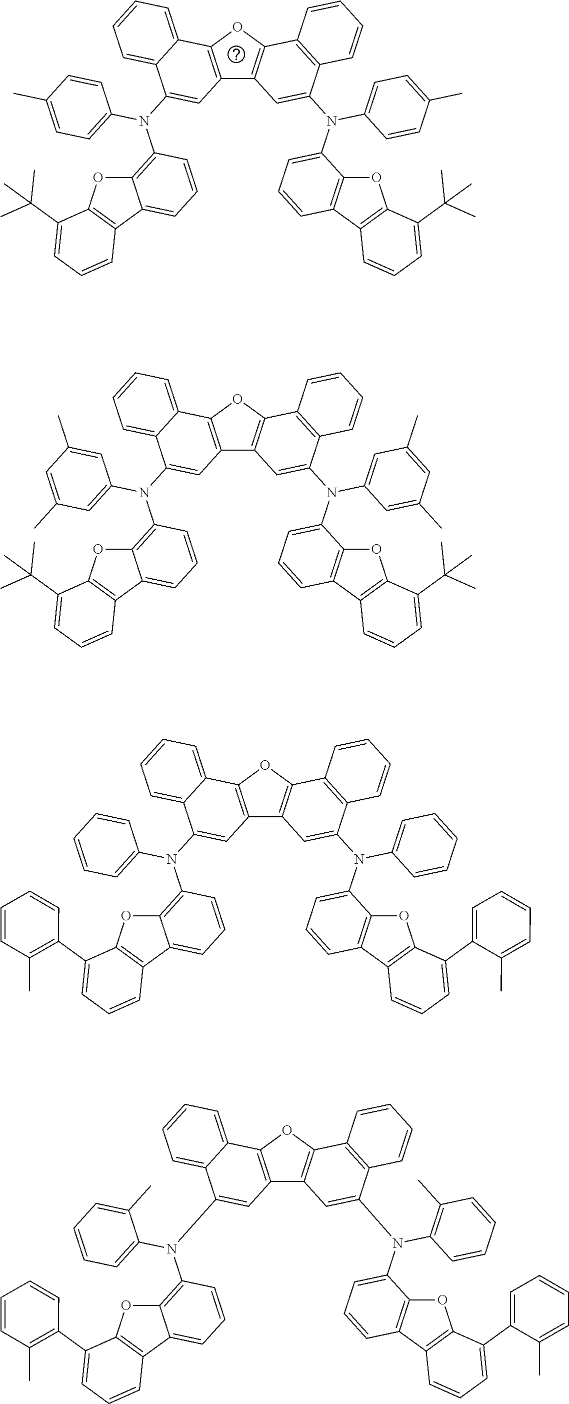

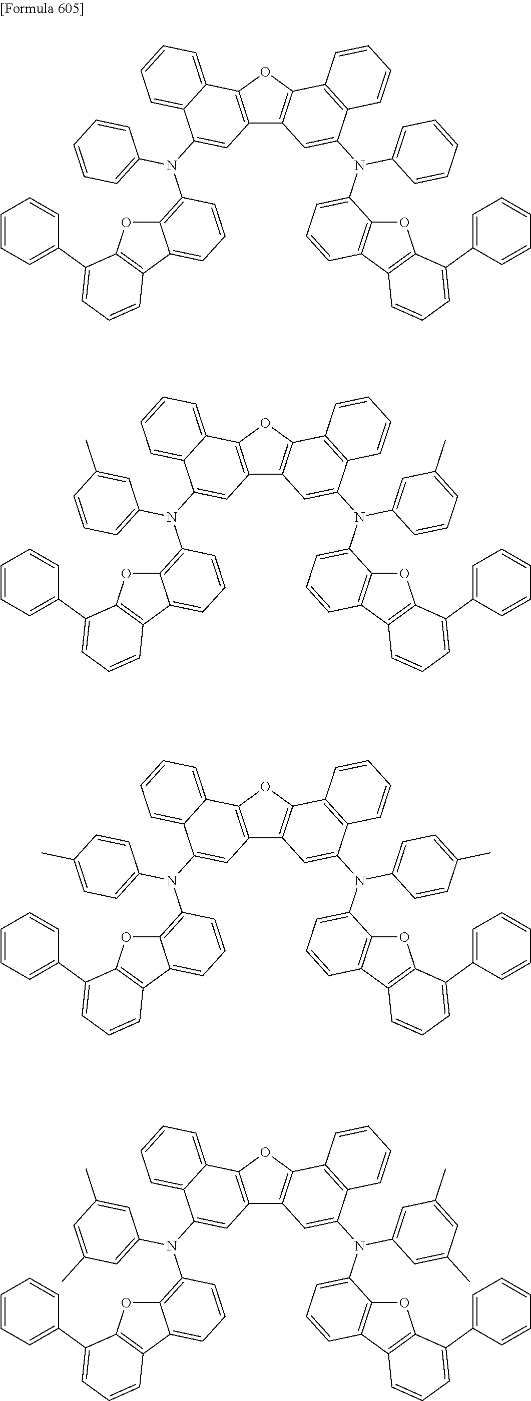 [SMILES: c1ccc(-c2cccc3c2oc2c(N(c4ccccc4)c4cc5c6cc(N(c7ccccc7)c7cccc8c7oc7c(-c9ccccc9)cccc78)c7ccccc7c6oc5c5ccccc45)cccc23)cc1 ; Cc1cccc(N(c2cc3c4cc(N(c5cccc(C)c5)c5cccc6c5oc5c(-c7ccccc7)cccc56)c5ccccc5c4oc3c3ccccc23)c2cccc3c2oc2c(-c4ccccc4)cccc23)c1 ; Cc1ccc(N(c2cc3c4cc(N(c5ccc(C)cc5)c5cccc6c5oc5c(-c7ccccc7)cccc56)c5ccccc5c4oc3c3ccccc23)c2cccc3c2oc2c(-c4ccccc4)cccc23)cc1 ; Cc1cc(C)cc(N(c2cc3c4cc(N(c5cc(C)cc(C)c5)c5cccc6c5oc5c(-c7ccccc7)cccc56)c5ccccc5c4oc3c3ccccc23)c2cccc3c2oc2c(-c4ccccc4)cccc23)c1]

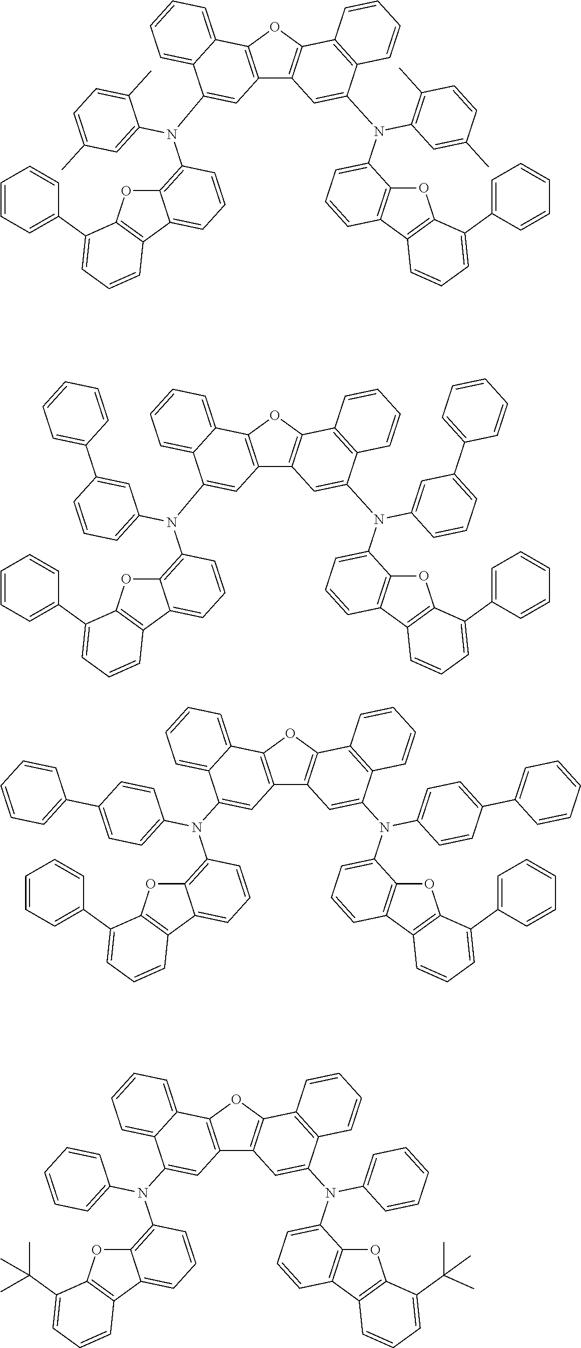

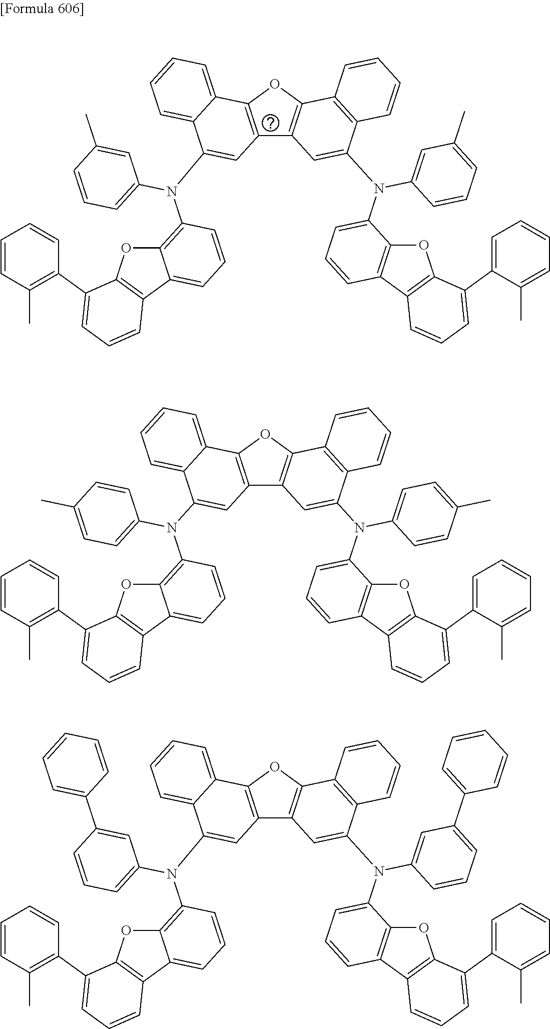 [SMILES: Cc1cccc(N(c2cc3c4cc(N(c5cccc(C)c5)c5cccc6c5oc5c(-c7ccccc7C)cccc56)c5ccccc5c4oc3c3ccccc23)c2cccc3c2oc2c(-c4ccccc4C)cccc23)c1 ; Cc1ccc(N(c2cc3c4cc(N(c5ccc(C)cc5)c5cccc6c5oc5c(-c7ccccc7C)cccc56)c5ccccc5c4oc3c3ccccc23)c2cccc3c2oc2c(-c4ccccc4C)cccc23)cc1 ; Cc1ccccc1-c1cccc2c1oc1c(N(c3cccc(-c4ccccc4)c3)c3cc4c5cc(N(c6cccc(-c7ccccc7)c6)c6cccc7c6oc6c(-c8ccccc8C)cccc67)c6ccccc6c5oc4c4ccccc34)cccc12]

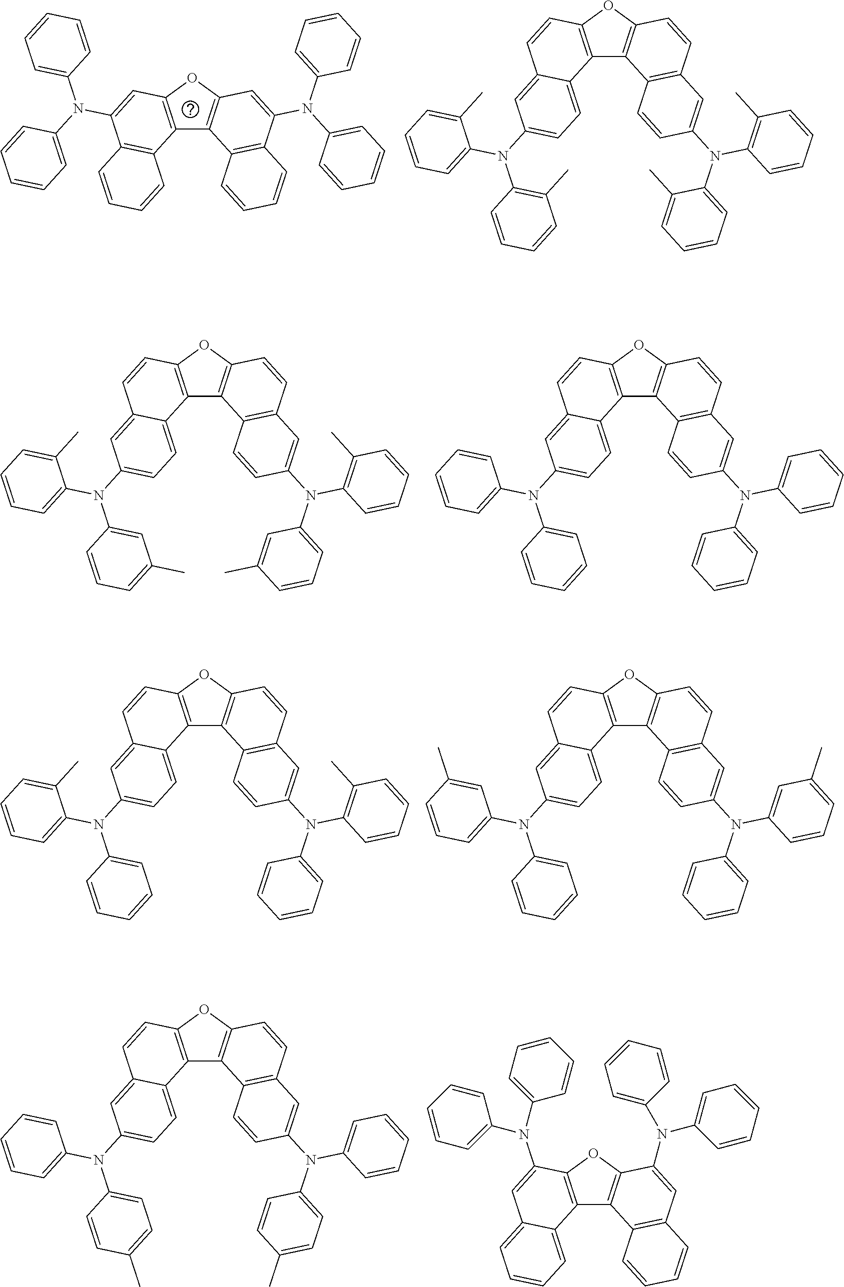

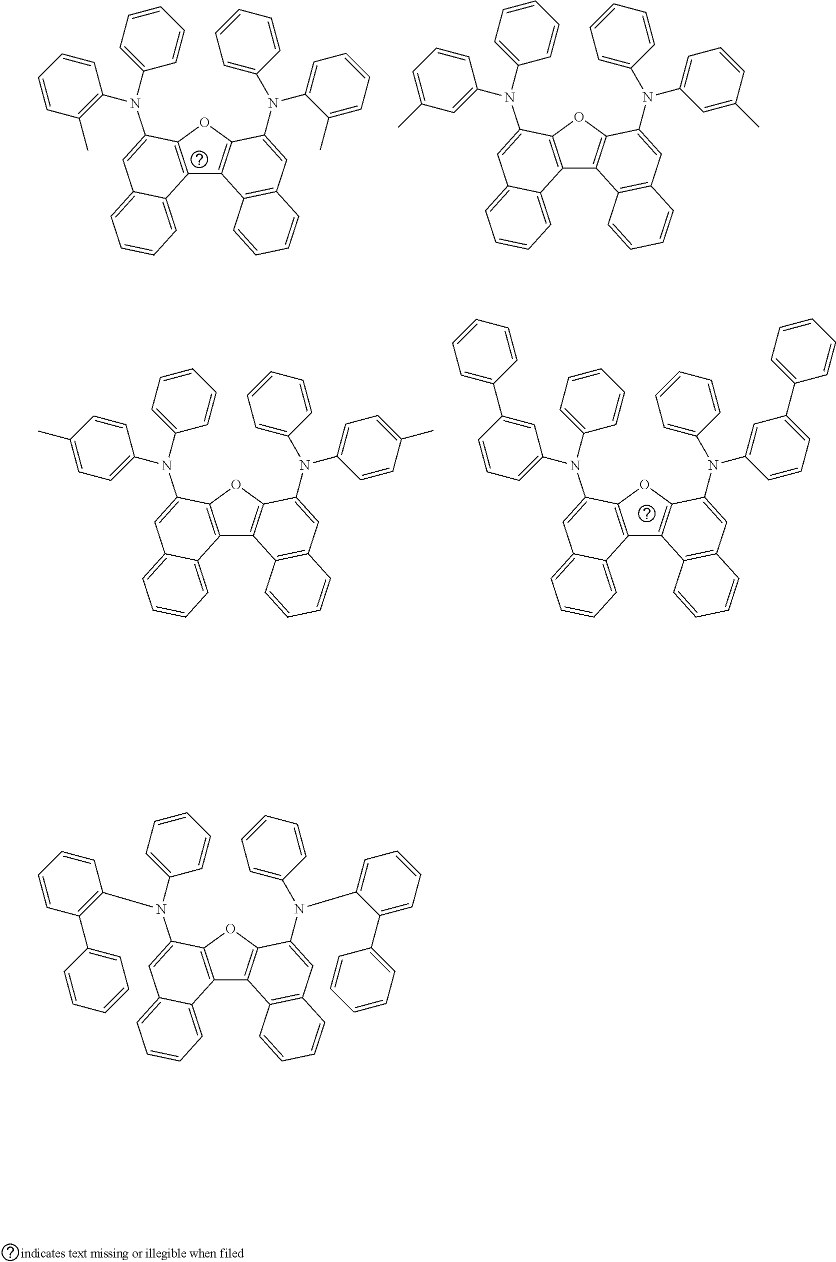

### Compound Represented by Formula (9)

The compound represented by the formula (9) will be described below.

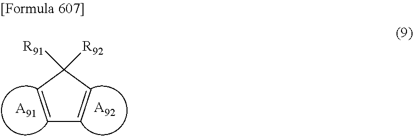

In the formula (9):
A91 ring and A92 ring are each independently a substituted or unsubstituted aromatic hydrocarbon ring having 6 to 50 ring carbon atoms, or a substituted or unsubstituted heterocycle having 5 to 50 ring atoms; and
at least one of A91 ring or A92 ring is bonded with * in a structure represented by a formula (92) below.

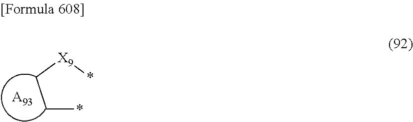

In the formula (92):
A₉₃ ring is a substituted or unsubstituted aromatic hydrocarbon ring having 6 to 50 ring carbon atoms, or a substituted or unsubstituted heterocycle having 5 to 50 ring atoms;
X₉ is NR₉₃, C(R₉₄)(R₉₅), Si(R₉₆)(R₉₇), Ge(R₉₈)(R₉₉), an oxygen atom, a sulfur atom, or a selenium atom;
R₉₁ and R₉₂ are mutually bonded to form a substituted or unsubstituted monocyclic ring, mutually bonded to form a substituted or unsubstituted fused ring, or not mutually bonded; and
R₉₁ and R₉₂ forming neither the monocyclic ring nor the fused ring, and R₉₃ to R₉₉ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

At least one ring selected from the group consisting of A91 ring and A92 ring is bonded to a bond * of a structure represented by the formula (92). In other words, the ring-forming carbon atoms of the aromatic hydrocarbon ring or the ring atoms of the heterocycle of the A91 ring in an exemplary embodiment are bonded to the bonds * in a structure represented by the formula (92). Further, the ring-forming carbon atoms of the aromatic hydrocarbon ring or the ring atoms of the heterocycle of the A92 ring in an exemplary embodiment are bonded to the bonds * in a structure represented by the formula (92).

In an exemplary embodiment, a group represented by a formula (93) below is bonded to one or both of the A91 ring and A92 ring.

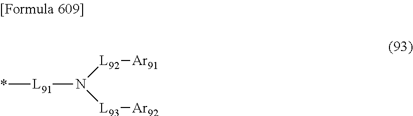

In the formula (93):
Ar₉₁ and Ar₉₂ are each independently a substituted or unsubstituted aryl group having 6 to 50 ring carbon atoms, or a substituted or unsubstituted heterocyclic group having 5 to 50 ring atoms;
L₉₁ to L₉₃ are each independently a single bond, a substituted or unsubstituted arylene group having 6 to 30 ring carbon atoms, a substituted or unsubstituted divalent heterocyclic group having 5 to 30 ring atoms, or a divalent linking group formed by bonding two, three or four groups selected from the group consisting of a substituted or unsubstituted arylene group having 6 to 30 ring carbon atoms and a substituted or unsubstituted divalent heterocyclic group having 5 to 30 ring atoms; and
* in the formula (93) represents a bonding position to one of A91 ring and A92 ring.

In an exemplary embodiment, in addition to the A91 ring, the ring-forming carbon atoms of the aromatic hydrocarbon ring or the ring atoms of the heterocycle of the A92 ring are bonded to * in a structure represented by the formula (92). In this case, the structures represented by the formula (92) may be mutually the same or different.

In an exemplary embodiment, R₉₁ and R₉₂ are each independently a substituted or unsubstituted aryl group having 6 to 50 ring carbon atoms.

In an exemplary embodiment, R₉₁ and R₉₂ are mutually bonded to form a fluorene structure.

In an exemplary embodiment, the rings A91 and A92 are each independently a substituted or unsubstituted aromatic hydrocarbon ring having 6 to 50 ring carbon atoms, example of which is a substituted or unsubstituted benzene ring.

In an exemplary embodiment, the ring A93 is a substituted or unsubstituted aromatic hydrocarbon ring having 6 to 50 ring carbon atoms, example of which is a substituted or unsubstituted benzene ring.

In an exemplary embodiment, X₉ is an oxygen atom or a sulfur atom.

Specific examples of the compound represented by the formula (9) include compounds shown below.

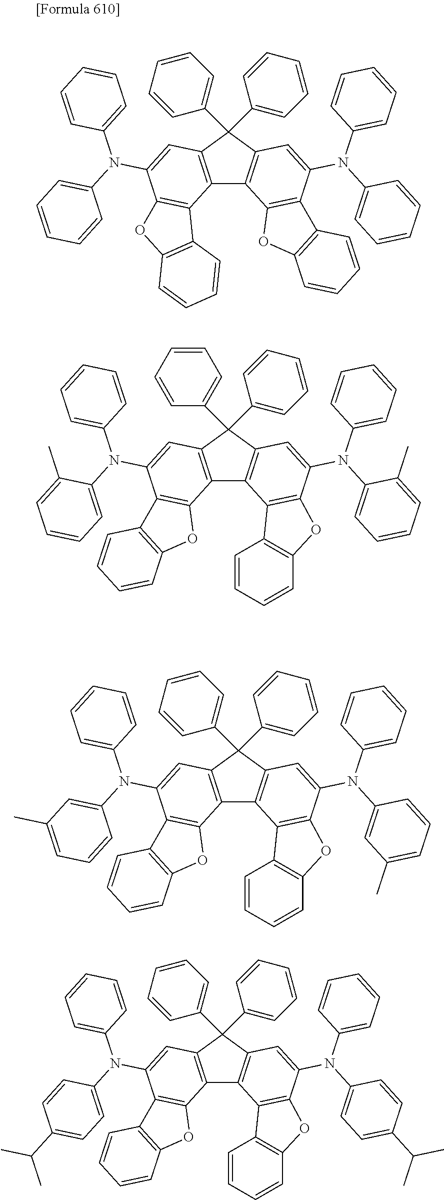

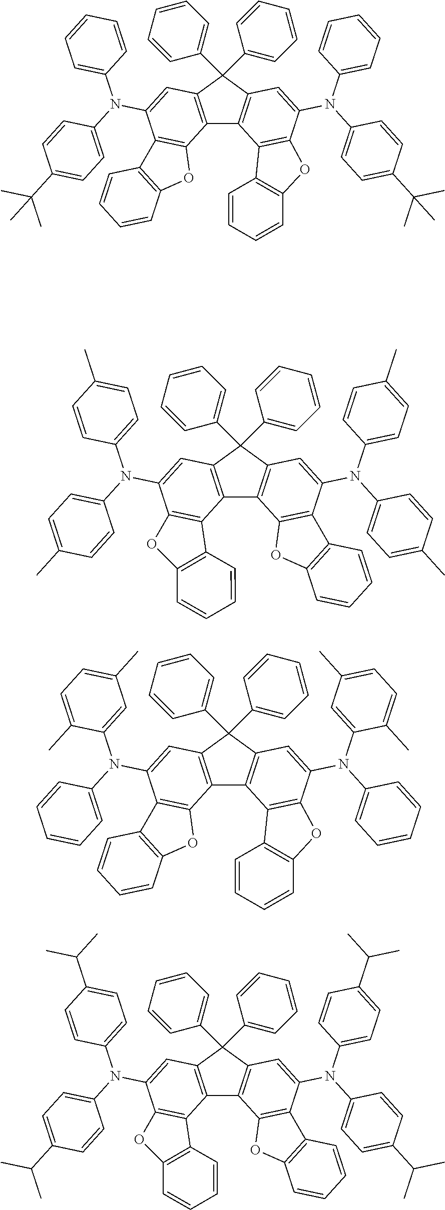

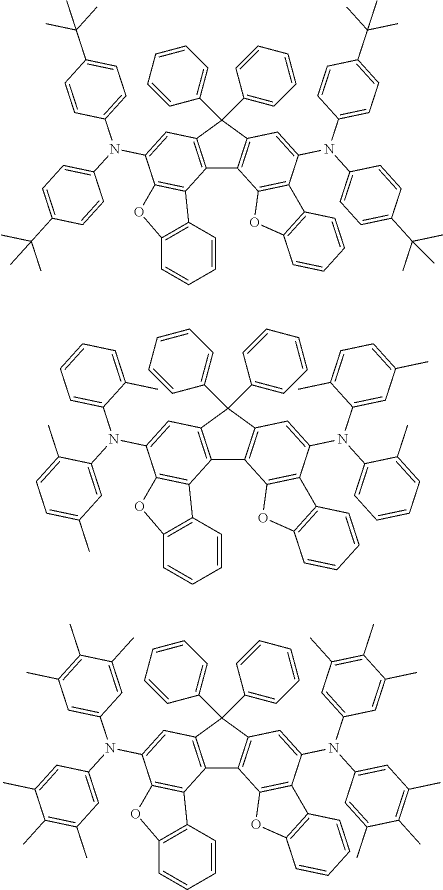

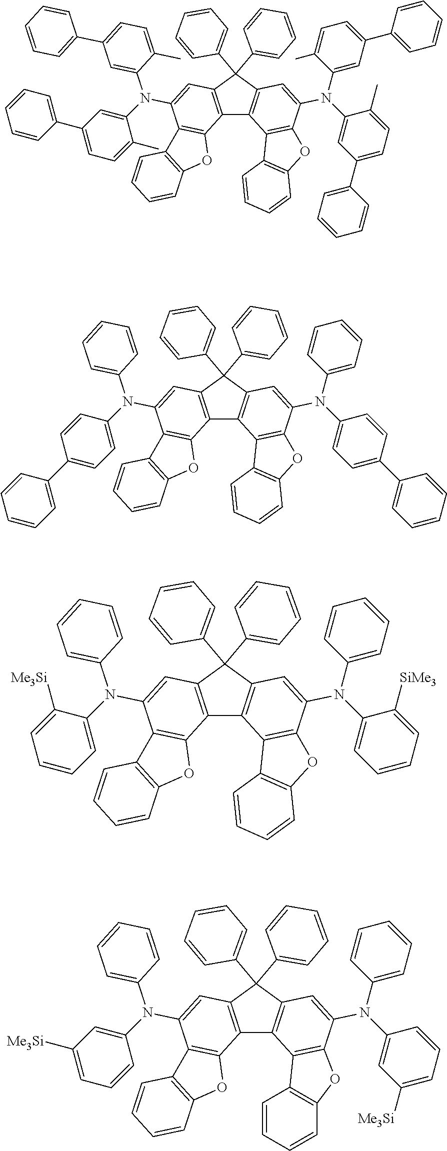

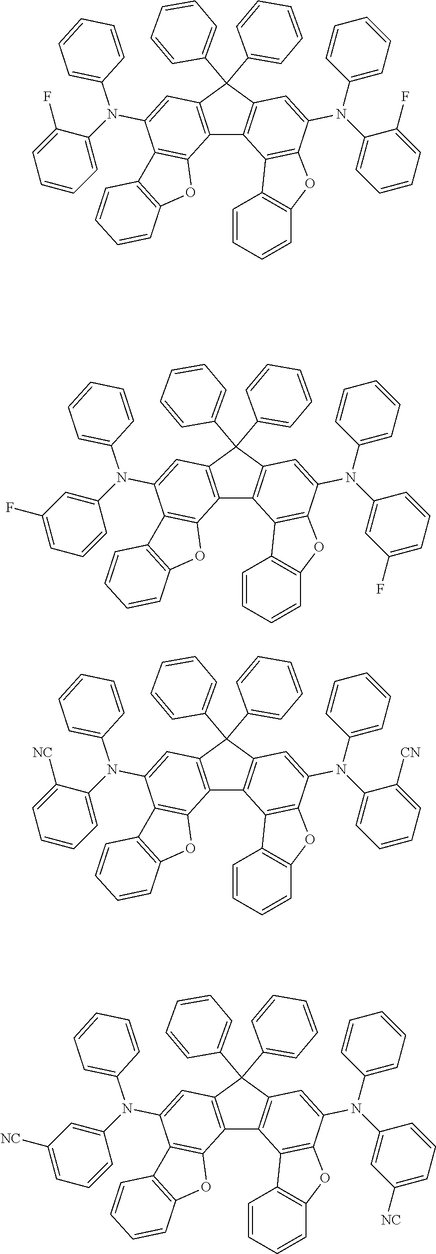

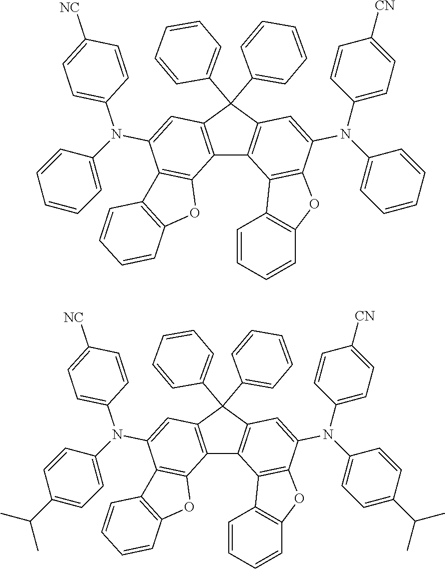 [SMILES: N#Cc1ccc(N(c2ccccc2)c2cc3c(c4c2oc2ccccc24)-c2c(cc(N(c4ccccc4)c4ccc(C#N)cc4)c4c2oc2ccccc24)C3(c2ccccc2)c2ccccc2)cc1 ; CC(C)c1ccc(N(c2ccc(C#N)cc2)c2cc3c(c4c2oc2ccccc24)-c2c(cc(N(c4ccc(C#N)cc4)c4ccc(C(C)C)cc4)c4c2oc2ccccc24)C3(c2ccccc2)c2ccccc2)cc1]

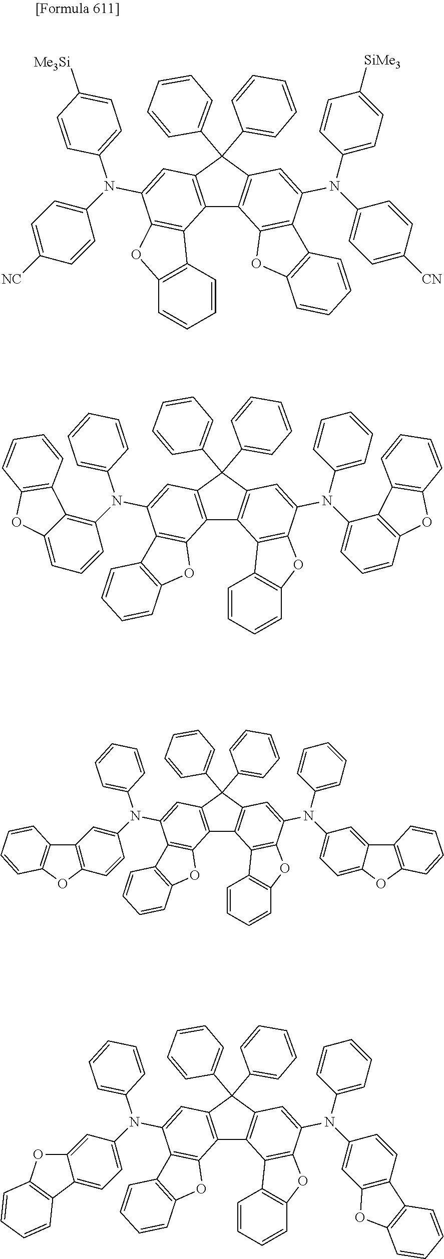

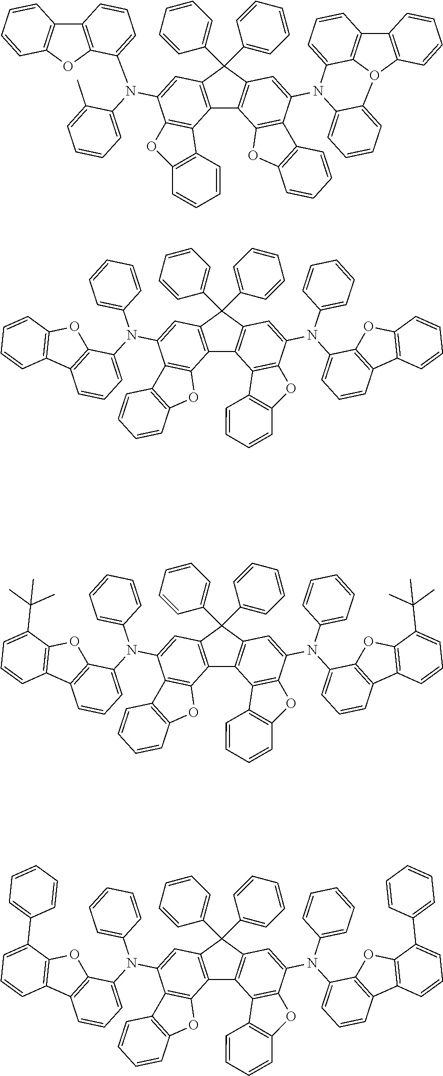

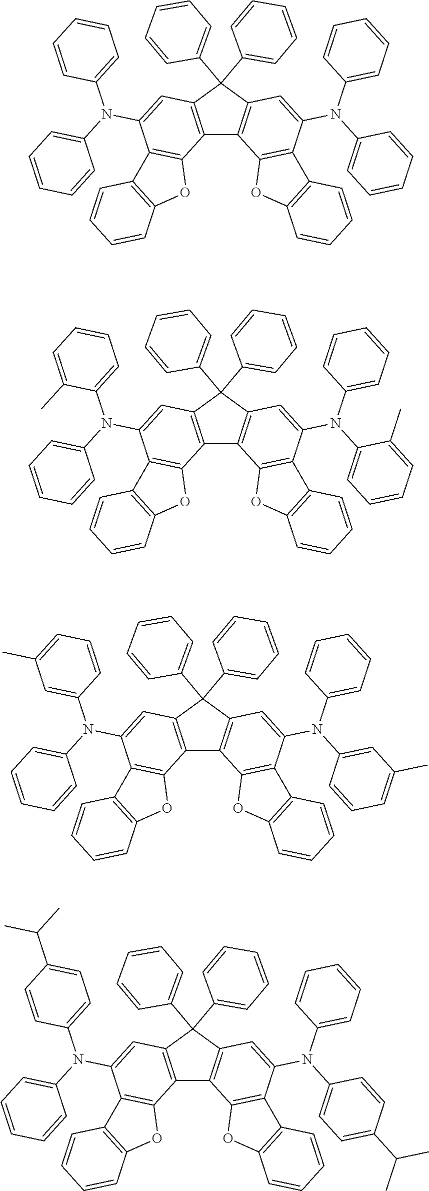

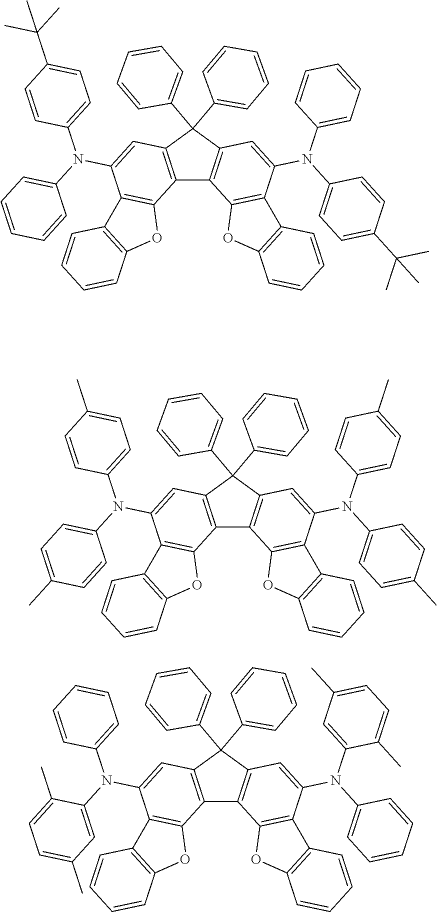

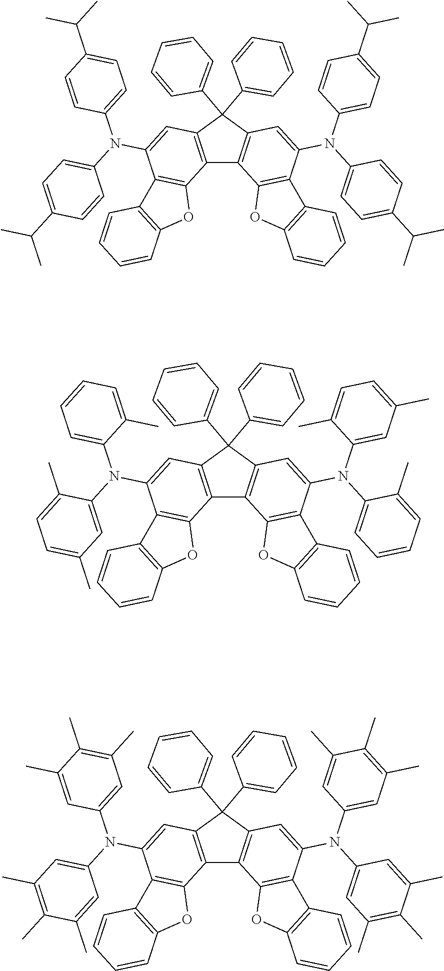

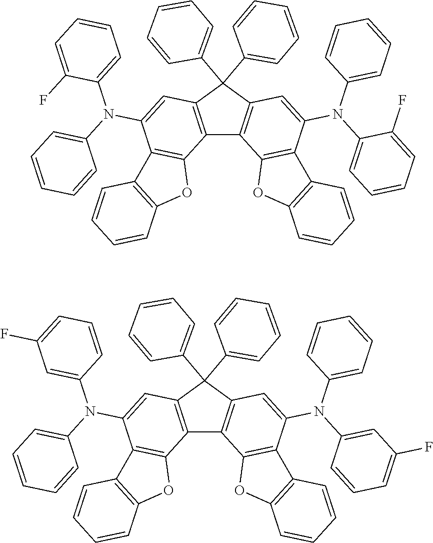 [SMILES: Fc1ccccc1N(c1ccccc1)c1cc2c(c3oc4ccccc4c13)-c1c(cc(N(c3ccccc3)c3ccccc3F)c3c1oc1ccccc13)C2(c1ccccc1)c1ccccc1 ; Fc1cccc(N(c2ccccc2)c2cc3c(c4oc5ccccc5c24)-c2c(cc(N(c4ccccc4)c4cccc(F)c4)c4c2oc2ccccc24)C3(c2ccccc2)c2ccccc2)c1]

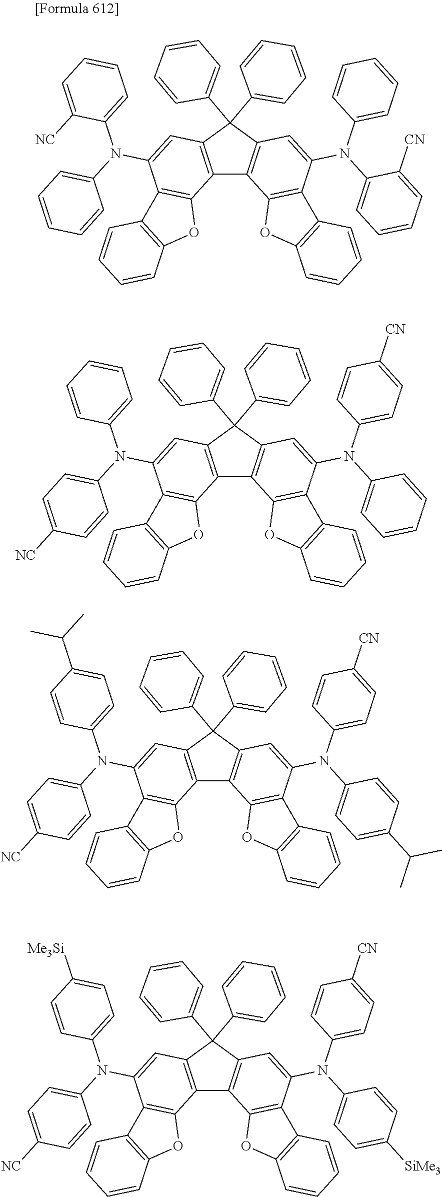

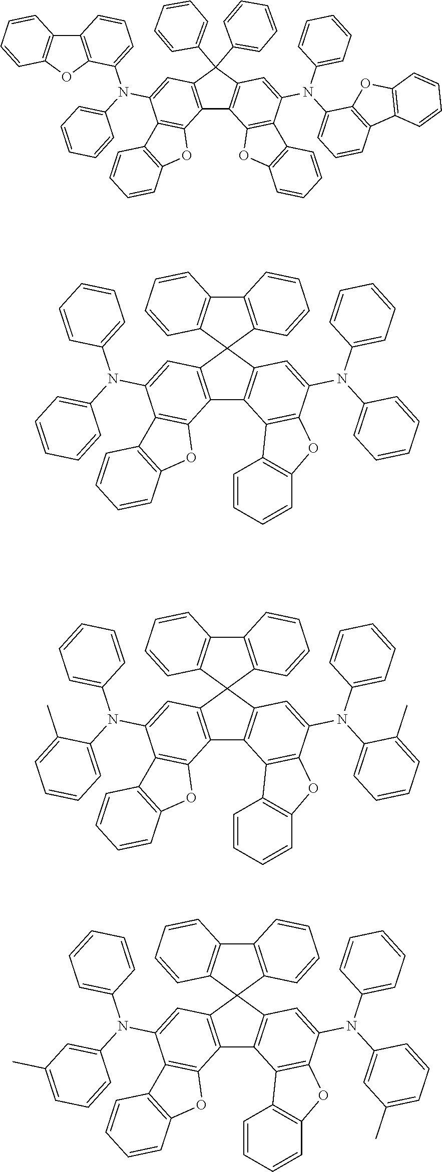 [SMILES: c1ccc(N(c2cccc3c2oc2ccccc23)c2cc3c(c4oc5ccccc5c24)-c2c(cc(N(c4ccccc4)c4cccc5c4oc4ccccc45)c4c2oc2ccccc24)C3(c2ccccc2)c2ccccc2)cc1 ; c1ccc(N(c2ccccc2)c2cc3c(c4c2oc2ccccc24)-c2c(cc(N(c4ccccc4)c4ccccc4)c4c2oc2ccccc24)C32c3ccccc3-c3ccccc32)cc1 ; Cc1ccccc1N(c1ccccc1)c1cc2c(c3c1oc1ccccc13)-c1c(cc(N(c3ccccc3)c3ccccc3C)c3c1oc1ccccc13)C21c2ccccc2-c2ccccc21 ; Cc1cccc(N(c2ccccc2)c2cc3c(c4c2oc2ccccc24)-c2c(cc(N(c4ccccc4)c4cccc(C)c4)c4c2oc2ccccc24)C32c3ccccc3-c3ccccc32)c1]

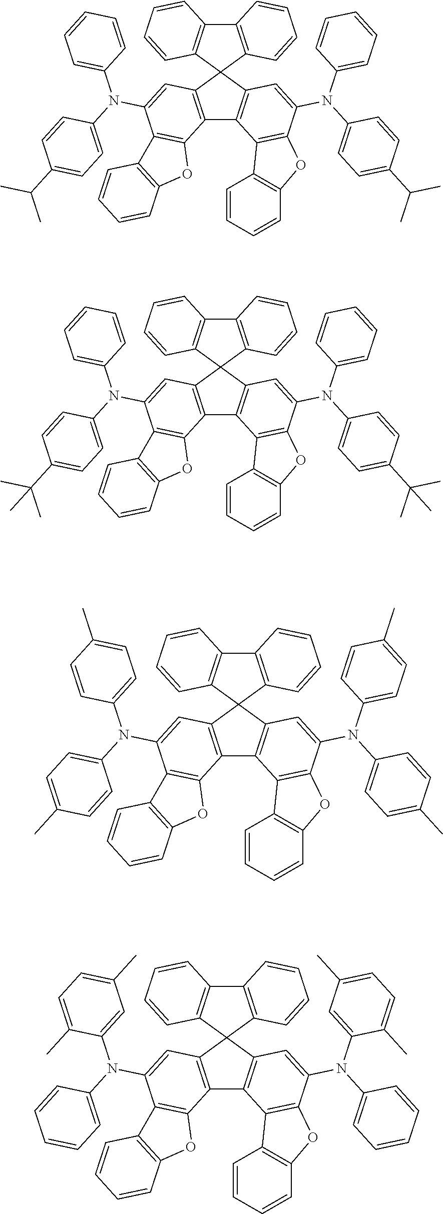

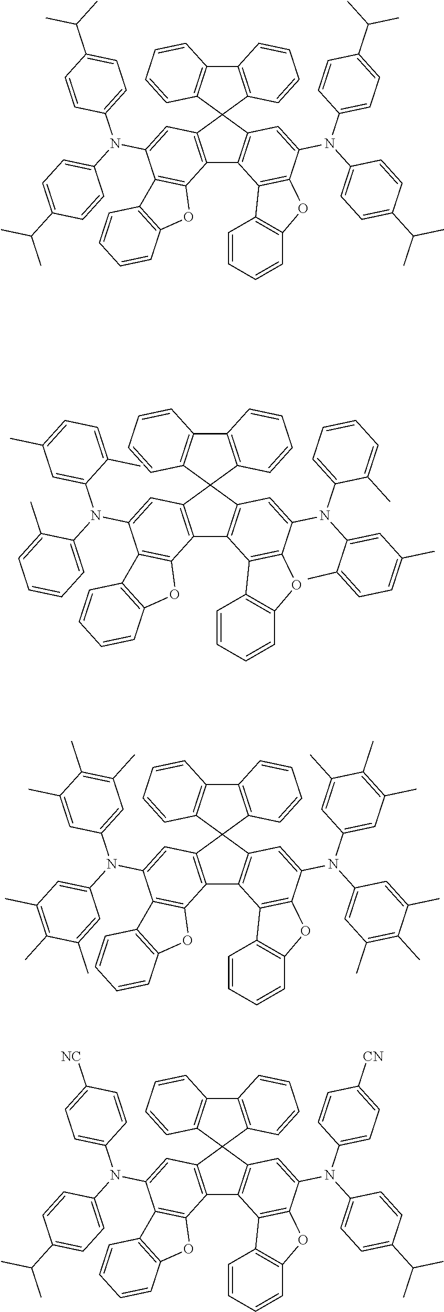

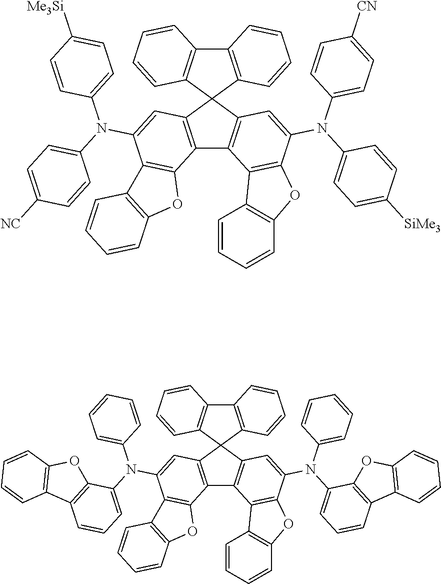 [SMILES: C[Si](C)(C)c1ccc(N(c2ccc(C#N)cc2)c2cc3c(c4c2oc2ccccc24)-c2c(cc(N(c4ccc(C#N)cc4)c4ccc([Si](C)(C)C)cc4)c4c2oc2ccccc24)C32c3ccccc3-c3ccccc32)cc1 ; c1ccc(N(c2cccc3c2oc2ccccc23)c2cc3c(c4c2oc2ccccc24)-c2c(cc(N(c4ccccc4)c4cccc5c4oc4ccccc45)c4c2oc2ccccc24)C32c3ccccc3-c3ccccc32)cc1]

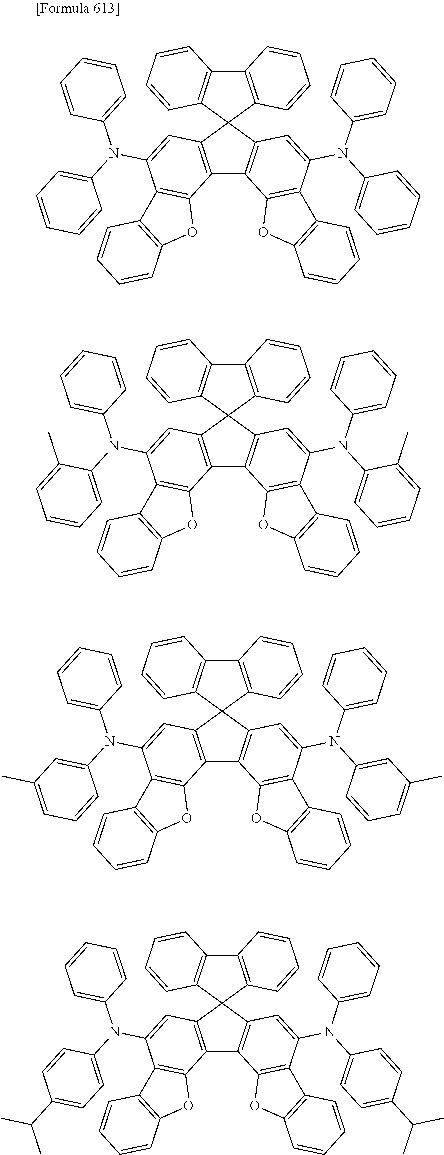

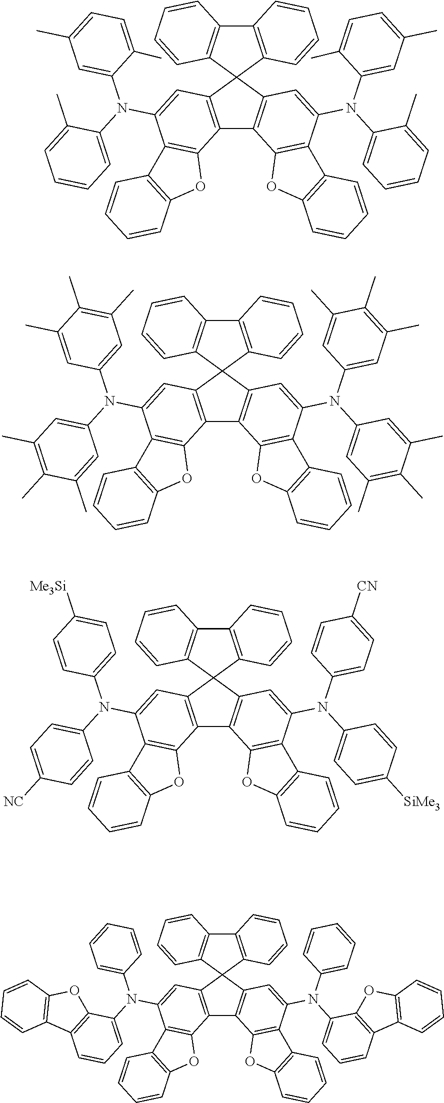

### Compound Represented by Formula (10)

The compound represented by the formula (10) will be described below.

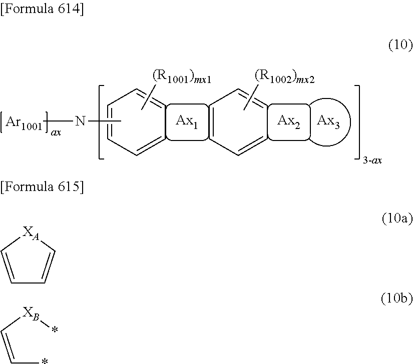

In the formula (10):

Ax₁ ring is a ring represented by the formula (10a) and fused with adjacent ring(s) at any position(s);
Ax₂ ring is a ring represented by the formula (10b) and fused with adjacent ring(s) at any position(s);
two * in the formula (10b) are bonded to Ax₃ ring at any position(s);
X_{A} and X_{B} are each independently C(R₁₀₀₃)(R₁₀₀₄), Si(R₁₀₀₅)(R₁₀₀₆), an oxygen atom or a sulfur atom;
Ax₃ ring is a substituted or unsubstituted aromatic hydrocarbon ring having 6 to 50 ring carbon atoms, or a substituted or unsubstituted heterocycle having 5 to 50 ring atoms;
Ar₁₀₀₁ is a substituted or unsubstituted aryl group having 6 to 50 ring carbon atoms, or a substituted or unsubstituted heterocyclic group having 5 to 50 ring atoms;
R₁₀₀₁ to R₁₀₀₆ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
mx1 is 3, mx2 is 2;
a plurality of R₁₀₀₁ are mutually the same or different;
a plurality of R₁₀₀₂ are mutually the same or different;
ax is 0, 1, or 2;
when ax is 0 or 1, the structures enclosed by brackets indicated by “3-ax” are mutually the same or different; and
when ax is 2, a plurality of Ar₁₀₀₁ are mutually the same or different.

In an exemplary embodiment, Ar₁₀₀₁ is a substituted or unsubstituted aryl group having 6 to 50 ring carbon atoms.

In an exemplary embodiment, Ax₃ ring is a substituted or unsubstituted aromatic hydrocarbon ring having 6 to 50 ring carbon atoms, example of which is a substituted or unsubstituted benzene ring, a substituted or unsubstituted naphthalene ring, or a substituted or unsubstituted anthracene ring.

In an exemplary embodiment, R₁₀₀₃ and R₁₀₀₄ are each independently a substituted or unsubstituted alkyl group having 1 to 50 carbon atoms.

In an exemplary embodiment, ax is 1.

Specific examples of the compound represented by the formula (10) include compounds shown below.

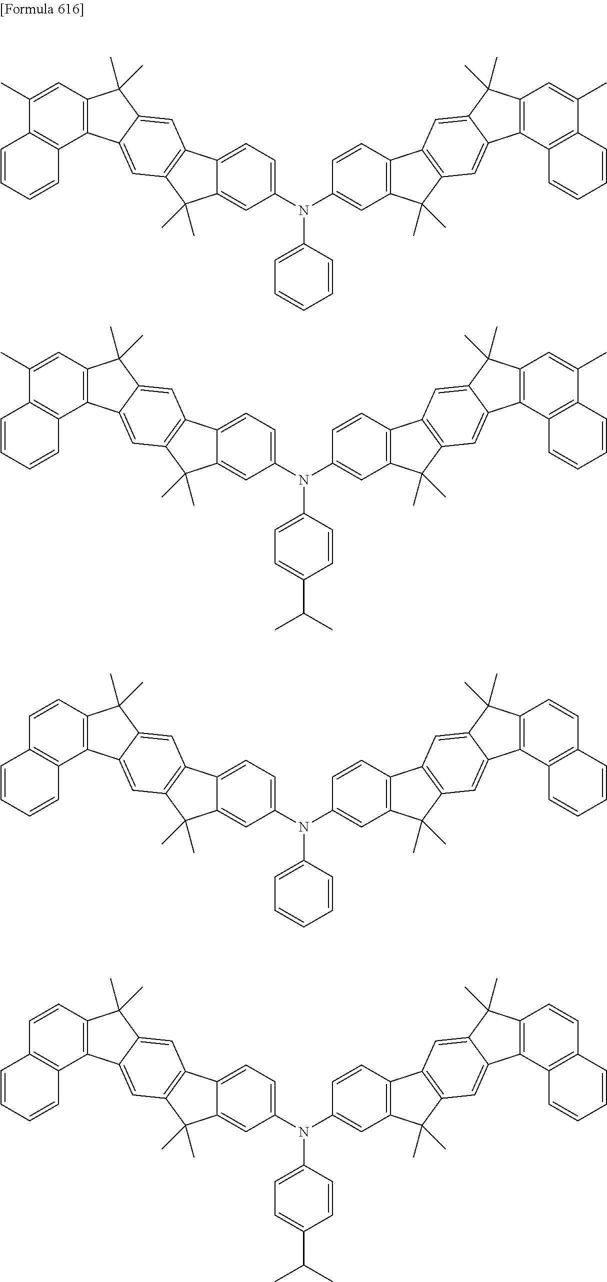

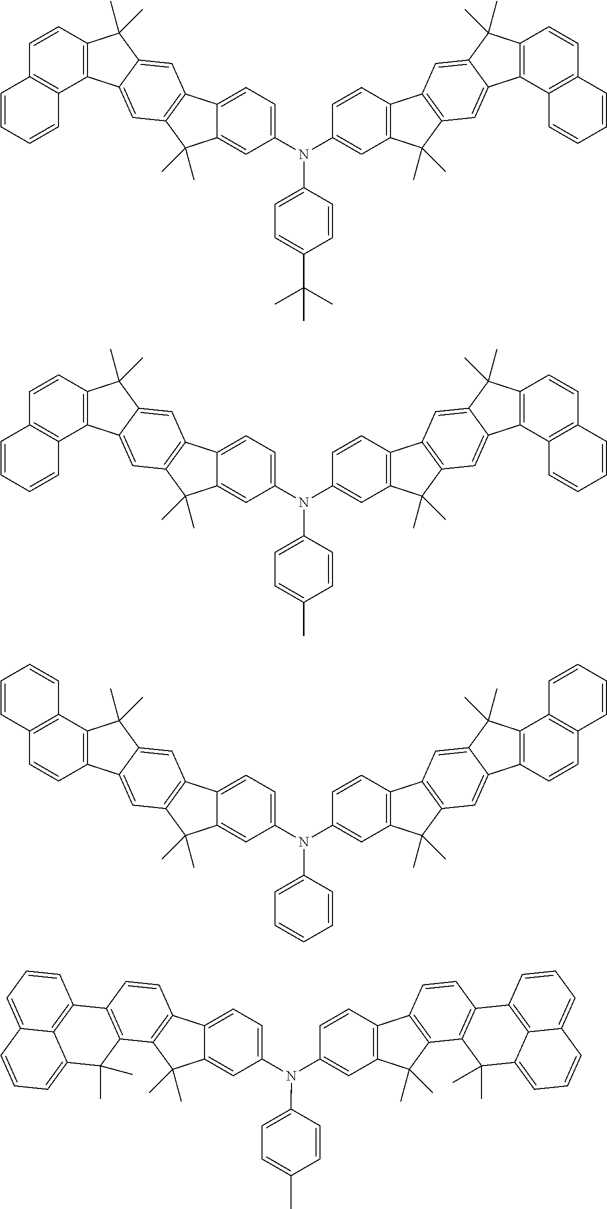 [SMILES: CC(C)(C)c1ccc(N(c2ccc3c(c2)C(C)(C)c2cc4c(cc2-3)C(C)(C)c2ccc3ccccc3c2-4)c2ccc3c(c2)C(C)(C)c2cc4c(cc2-3)C(C)(C)c2ccc3ccccc3c2-4)cc1 ; Cc1ccc(N(c2ccc3c(c2)C(C)(C)c2cc4c(cc2-3)C(C)(C)c2ccc3ccccc3c2-4)c2ccc3c(c2)C(C)(C)c2cc4c(cc2-3)C(C)(C)c2ccc3ccccc3c2-4)cc1 ; CC1(C)c2cc(N(c3ccccc3)c3ccc4c(c3)C(C)(C)c3cc5c(cc3-4)C(C)(C)c3c-5ccc4ccccc34)ccc2-c2cc3c(cc21)-c1ccc2ccccc2c1C3(C)C ; Cc1ccc(N(c2ccc3c(c2)C(C)(C)c2c-3ccc3c2C(C)(C)c2cccc4cccc-3c24)c2ccc3c(c2)C(C)(C)c2c-3ccc3c2C(C)(C)c2cccc4cccc-3c24)cc1]

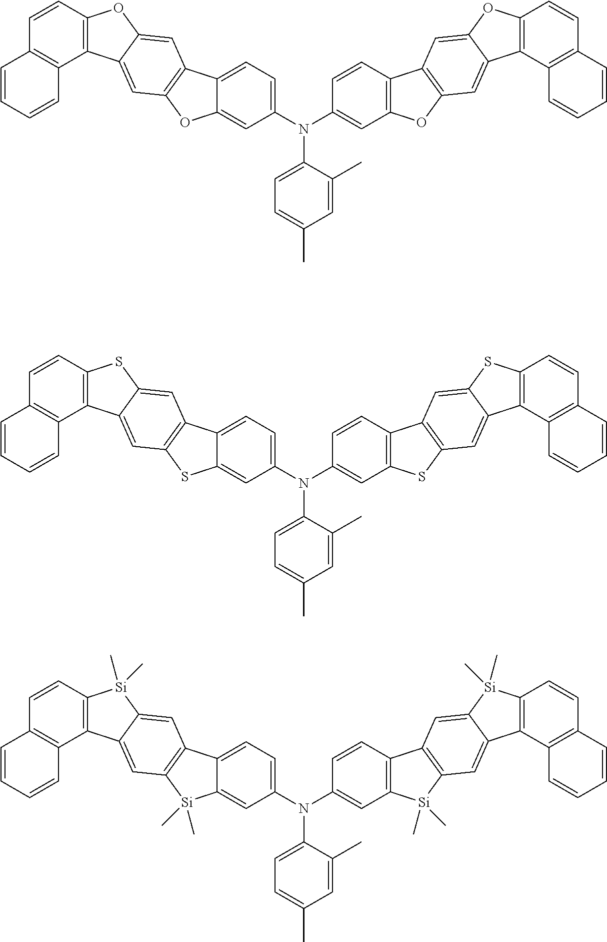 [SMILES: Cc1ccc(N(c2ccc3c(c2)oc2cc4c(cc23)oc2ccc3ccccc3c24)c2ccc3c(c2)oc2cc4c(cc23)oc2ccc3ccccc3c24)c(C)c1 ; Cc1ccc(N(c2ccc3c(c2)sc2cc4c(cc23)sc2ccc3ccccc3c24)c2ccc3c(c2)sc2cc4c(cc23)sc2ccc3ccccc3c24)c(C)c1 ; Cc1ccc(N(c2ccc3c(c2)[Si](C)(C)c2cc4c(cc2-3)[Si](C)(C)c2ccc3ccccc3c2-4)c2ccc3c(c2)[Si](C)(C)c2cc4c(cc2-3)[Si](C)(C)c2ccc3ccccc3c2-4)c(C)c1]

In an exemplary embodiment, the emitting layer contains, as at least one of the third compound or the fourth compound, at least one compound selected from the group consisting of a compound represented by the formula (4), a compound represented by the formula (5), a compound represented by the formula (7), a compound represented by the formula (8), a compound represented by the formula (9), and a compound represented by a formula (63a ) below.

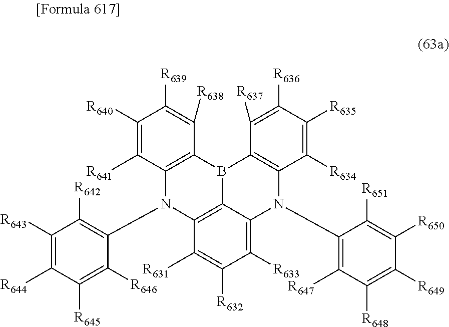

In the formula (63a ):
R₆₃₁ is bonded with R₆₄₆ to form a substituted or unsubstituted heterocycle, or not bonded therewith to form no substituted or unsubstituted heterocycle;
R₆₃₃ is bonded with R₆₄₇ to form a substituted or unsubstituted heterocycle, or not bonded therewith to form no substituted or unsubstituted heterocycle;
R₆₃₄ is bonded with R₆₅₁ to form a substituted or unsubstituted heterocycle, or not bonded therewith to form no substituted or unsubstituted heterocycle;
R₆₄₁ is bonded with R₆₄₂ to form a substituted or unsubstituted heterocycle, or not bonded therewith to form no substituted or unsubstituted heterocycle;
at least one combination of adjacent two or more of R₆₃₁ to R₆₅₁ are mutually bonded to form a substituted or unsubstituted monocyclic ring, mutually bonded to form a substituted or unsubstituted fused ring, or not mutually bonded;
R₆₃₁ to R₆₅₁ not forming the substituted or unsubstituted heterocycle, not forming the monocyclic ring, and not forming the fused ring are each independently a hydrogen atom, a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and
at least one of R₆₃₁ to R₆₅₁ not forming the substituted or unsubstituted heterocycle, not forming the monocyclic ring and not forming the fused ring are a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, the compound represented by the formula (4) is a compound represented by the formula (41-3), the formula (41-4), or the formula (41-5), the A1 ring in the formula (41-5) being a substituted or unsubstituted fused aromatic hydrocarbon ring having 10 to 50 ring carbon atoms, or a substituted or unsubstituted fused heterocycle having 8 to 50 ring atoms.

In an exemplary embodiment, the substituted or unsubstituted fused aromatic hydrocarbon ring having 10 to 50 ring carbon atoms in the formulae (41-3), (41-4) and (41-5) is a substituted or unsubstituted naphthalene ring, a substituted or unsubstituted anthracene ring, or a substituted or unsubstituted fluorene ring; and
the substituted or unsubstituted fused heterocycle having 8 to 50 ring atoms is a substituted or unsubstituted dibenzofuran ring, a substituted or unsubstituted carbazole ring, or a substituted or unsubstituted dibenzothiophene ring.

In an exemplary embodiment, the substituted or unsubstituted fused aromatic hydrocarbon ring having 10 to 50 ring carbon atoms in the formula (41-3), (41-4) or (41-5) is a substituted or unsubstituted naphthalene ring, or a substituted or unsubstituted fluorene ring; and
the substituted or unsubstituted fused heterocycle having 8 to 50 ring atoms is a substituted or unsubstituted dibenzofuran ring, a substituted or unsubstituted carbazole ring, or a substituted or unsubstituted dibenzothiophene ring.

In an exemplary embodiment, the compound represented by the formula (4) is selected from the group consisting of a compound represented by a formula (461) below, a compound represented by a formula (462) below, a compound represented by a formula (463) below, a compound represented by a formula (464) below, a compound represented by a formula (465) below, a compound represented by a formula (466) below, and a compound represented by a formula (467) below.

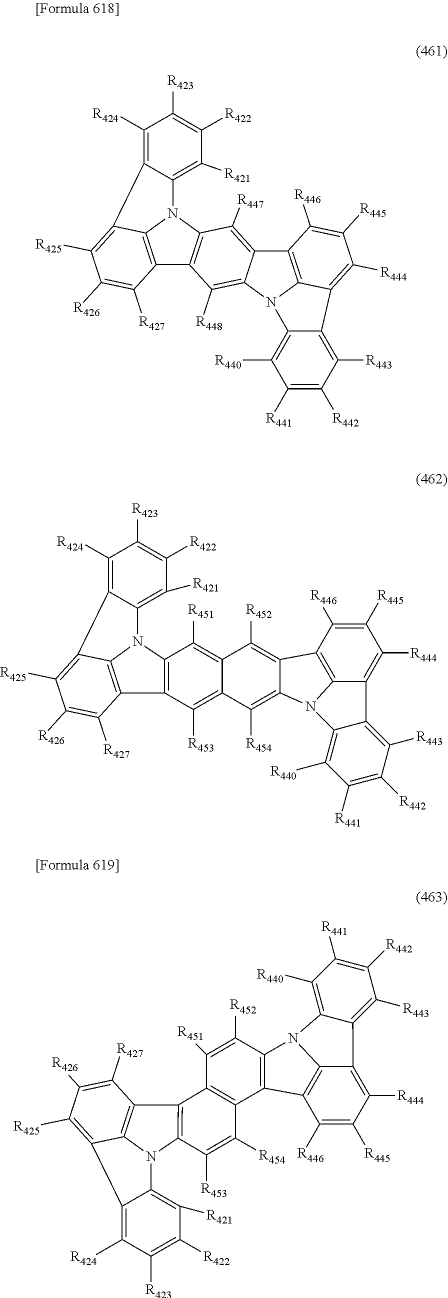

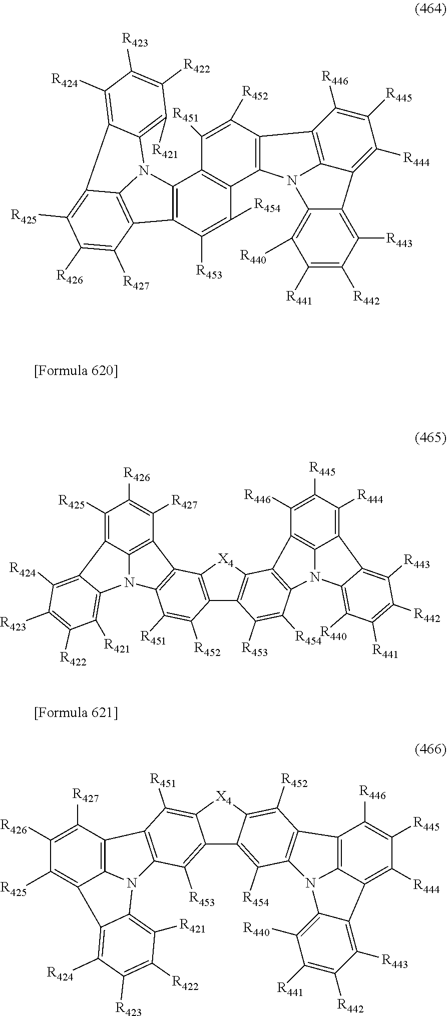

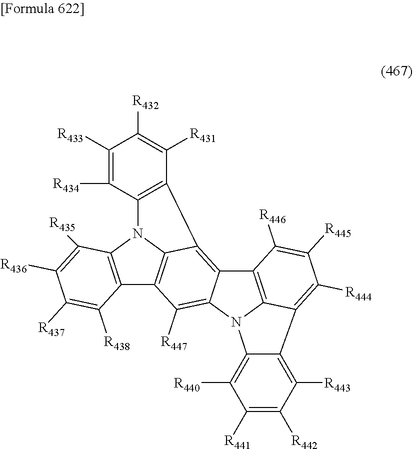

In the formulae (461) to (467):
at least one combination of adjacent two or more of R₄₂₁ to R₄₂₇, R₄₃₁ to R₄₃, R₄₄₀ to R₄₄, and R₄₁ to R₄₅₄ are mutually bonded to form a substituted or unsubstituted monocyclic ring, mutually bonded to form a substituted or unsubstituted fused ring, or not mutually bonded;
R₄₃₇, R₄₃₅, and R₄₂₁ to R₄₂₇, R₄₃₁ to R₄₃, R₄₄₀ to R₄₄₅, and R₄₅₁ to R₄₅₄ forming neither the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
X₄ is an oxygen atom, NR₈₀₁, or C(R₈₀₂)(R₈₀₃);
R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
a substituted or unsubstituted alkyl group having 1 to 50 carbon atoms, or a substituted or unsubstituted aryl group having 6 to 50 ring carbon atoms;
when a plurality of R₈₀₁ are present, the plurality of R₈₀₁ are mutually the same or different;
when a plurality of R₈₀₂ are present, the plurality of R₈₀₂ are mutually the same or different; and
when a plurality of R₈₀₃ are present, the plurality of R₈₀₃ are mutually the same or different.

In an exemplary embodiment, R₄₂₁ to R₄₂₇ and R₄₄₀ to R₄₄₅ are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₄₂₁ to R₄₂₇ and R₄₄₀ to R₄₄₇ are each independently selected from the group consisting of a hydrogen atom, a substituted or unsubstituted aryl group having 6 to 18 ring carbon atoms, and a substituted or unsubstituted heterocyclic group having 5 to 18 ring atoms.

In an exemplary embodiment, the compound represented by the formula (41-3) is a compound represented by a formula (41-3-1) below.

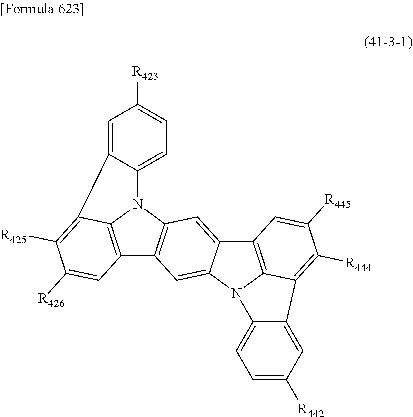

In the formula (41-3-1), R₄₂₃, R₄₂₅, R₄₂₆, R₄₄₂, R₄₄₄ and R₄₄₅ each independently represent the same as R₄₂₃, R₄₂₅, R₄₂₆, R₄₄₂, R₄₄₄ and R₄₄₅ in the formula (41-3).

In an exemplary embodiment, the compound represented by the formula (41-3) is a compound represented by a formula (41-3-2) below.

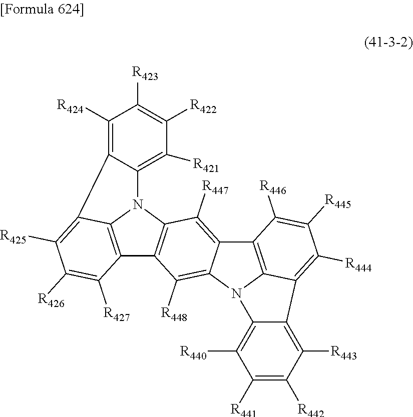

In the formula (41-3-2):
R₄₂₁ to R₄₂₇ and R₄₄₀ to R₄₄₈ each independently represent the same as R₄₂₁ to R₄₂₇ and R₄₄₀ to R₄₄₈ in the formula (41-3); and
at least one of R₄₂₁ to R₄₂₇ or R₄₄₀ to R₄₄₆ is a group represented by —N(R₉₀₆)(R₉₀₇).

In an exemplary embodiment, two of R₄₂₁ to R₄₂₇ and R₄₄₀ to R₄₄₆ in the formula (41-3-2) are each a group represented by —N(R₉₀₆)(R₉₀₇).

In an exemplary embodiment, the compound represented by the formula (41-3-2) is a compound represented by a formula (41-3-3) below.

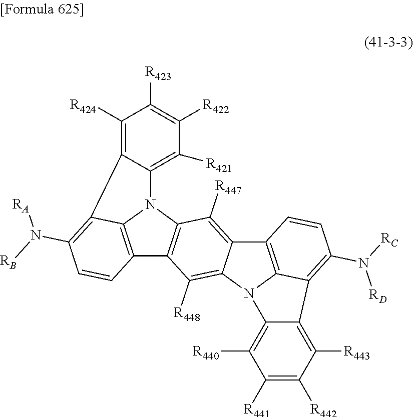

In the formula (41-3-3):
R₄₂₁ to R₄₂₄, R₄₄₀ to R₄₄₃, R₄₄₇, and R₄₄₈ each independently represent the same as R₄₂₁ to R₄₂₄, R₄₄₀ to R₄₄₃, R₄₄₇, and R₄₄₈ in the formula (41-3); and
R_{A}, R_{B}, R_{C}, and R_{D} are each independently a substituted or unsubstituted aryl group having 6 to 18 ring carbon atoms, or a substituted or unsubstituted heterocyclic group having 5 to 18 ring atoms.

In an exemplary embodiment, the compound represented by the formula (41-3-3) is a compound represented by a formula (41-3-4) below.

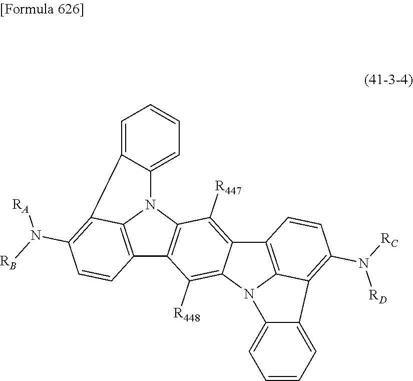

In the formula (41-3-4), R₄₄₇, R₄₄₈, R_{A}, R_{B}, R_{C} and R_{D} each independently represent the same as R₄₄₇, R₄₄₈, R_{A}, R_{B}, R_{C} and R_{D} in the formula (41-3-3).

In an exemplary embodiment, R_{A}, R_{B}, R_{C}, and R_{D} are each independently a substituted or unsubstituted aryl group having 6 to 18 ring carbon atoms.

In an exemplary embodiment, R_{A}, R_{B}, R_{C}, and R_{D} are each independently a substituted or unsubstituted phenyl group.

In an exemplary embodiment, R₄₄₇ and R₄₄₈ are each a hydrogen atom.

In an exemplary embodiment, the substituent for “the substituted or unsubstituted” group in each of the formulae is an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁ₐ)(R₉₀₂ₐ)(R₉₀₃ₐ), —O—(R₉₀₄ₐ), —S—(R₉₀₅ₐ), —N(R₉₀₆ₐ)(R₉₀₇ₐ), a halogen atom, a cyano group, a nitro group, an unsubstituted aryl group having 6 to 50 ring carbon atoms, or an unsubstituted heterocyclic group having 5 to 50 ring atoms;
R₉₀₁ₐ to R₉₀₇ₐ are each independently a hydrogen atom, an unsubstituted alkyl group having 1 to 50 carbon atoms, an unsubstituted aryl group having 6 to 50 ring carbon atoms, or an unsubstituted heterocyclic group having 5 to 50 ring atoms;
when two or more R₉₀₁ₐ are present, the two or more R₉₀₁ₐ are mutually the same or different;
when two or more R₉₀₂ₐ are present, the two or more R₉₀₂ₐ are mutually the same or different;
when two or more R₉₀₃ₐ are present, the two or more R₉₀₃ₐ are mutually the same or different;
when two or more R₉₀₄ₐ are present, the two or more R₉₀₄ₐ are mutually the same or different;
when two or more R₉₀₅ₐ are present, the two or more R₉₀₅ₐ are mutually the same or different;
when two or more R₉₀₅ₐ are present, the two or more R₉₀₅ₐ are mutually the same or different; and
when two or more R₉₀₇ₐ are present, the two or more R₉₀₇ₐ are mutually the same or different.

In an exemplary embodiment, the substituent “for the substituted or unsubstituted” group in each of the formulae is an unsubstituted alkyl group having 1 to 50 carbon atoms, an unsubstituted aryl group having 6 to 50 ring carbon atoms, or an unsubstituted heterocyclic group having 5 to 50 ring atoms.

In an exemplary embodiment, the substituent for “the substituted or unsubstituted” group in each of the formulae is an unsubstituted alkyl group having 1 to 18 carbon atoms, an unsubstituted aryl group having 6 to 18 ring carbon atoms, or an unsubstituted heterocyclic group having 5 to 18 ring atoms.

In the organic electroluminescence device according to the exemplary embodiment, it is preferable that the first emitting layer further contains the third compound; and the third compound is a compound that emits light having a maximum peak wavelength in a range from 430 nm to 480 nm. The third compound in the first emitting layer is more preferably a compound that emits fluorescence. The third compound is more preferably a compound that emits fluorescence having a maximum peak wavelength in a range from 430 nm to 480 nm.

In the organic electroluminescence device according to the exemplary embodiment, it is preferable that the second emitting layer further contains the fourth compound; and the fourth compound is a compound that emits light having a maximum peak wavelength in a range from 430 nm to 480 nm. The fourth compound in the second emitting layer is more preferably a compound that emits fluorescence. The fourth compound is more preferably a compound that emits fluorescence having a maximum peak wavelength in a range from 430 nm to 480 nm.

A measurement method of the maximum peak wavelength of a compound is as follows. A toluene solution of a measurement target compound at a concentration ranging from 10⁻⁶ mol/L to 10⁻⁵ mol/L is prepared and put in a quartz cell. An emission spectrum (ordinate axis: luminous intensity, abscissa axis: wavelength) of the thus-obtained sample is measured at a normal temperature (300K). The emission spectrum is measurable using a spectrophotometer (machine name: F-7000) manufactured by Hitachi High-Tech Science Corporation. It should be noted that the machine for measuring the emission spectrum is not limited to the machine used herein.

A peak wavelength of the emission spectrum exhibiting the maximum luminous intensity is defined as a maximum peak wavelength. Herein, the maximum peak wavelength of fluorescence is occasionally referred to as a maximum fluorescence peak wavelength (FL-peak).

In the organic electroluminescence device according to the exemplary embodiment, the groups specified to be “substituted or unsubstituted” are each preferably an “unsubstituted” group.

In the first compound and the second compound according to the exemplary embodiment, the groups specified to be “substituted or unsubstituted” are each preferably an “unsubstituted” group.

In the organic electroluminescence device according to the exemplary embodiment, the first compound is preferably a host material. The first compound as a host material is occasionally referred to as a first host material.

When the first emitting layer of the organic EL device according to the exemplary embodiment contains the first compound and the third compound, the first compound is preferably a host material (occasionally also referred to as a matrix material) and the third compound is preferably a dopant material (occasionally also referred to as a guest material, emitter or luminescent material).

When the first emitting layer of the organic EL device according to the exemplary embodiment contains the first compound and the third compound, a singlet energy S₁(H1) of the first compound and a singlet energy S₁(D3) of the third compound preferably satisfy a relationship of a numerical formula (Numerical Formula 1) below.

S₁(H1)>S₁(D3) (Numerical Formula 1)

The singlet energy S₁ means an energy difference between the lowest singlet state and the ground state.

In the organic electroluminescence device according to the exemplary embodiment, the second compound is preferably a host material. The second compound as a host material is occasionally referred to as a second host material.

In the organic EL device according to the exemplary embodiment, when the second emitting layer contains the second compound and the fourth compound, the second compound is preferably a host material (occasionally also referred to as a matrix material) and the fourth compound is preferably a dopant material (occasionally also referred to as a guest material, emitter or luminescent material).

When the second emitting layer of the organic EL device according to the exemplary embodiment contains the second compound and the fourth compound, a singlet energy S₁(H2) of the second compound and a singlet energy S₁(D4) of the fourth compound preferably satisfy a relationship of a numerical formula (Numerical Formula 2) below.

S₁(H2)>S₁(D4) (Numerical Formula 2)

In the organic EL device according to the exemplary embodiment, it is also preferable that the first emitting layer contains the first host material as the first compound and a dopant material as the third compound (occasionally referred to as a first dopant material); and the second emitting layer contains the second host material as the second compound and a dopant material as the fourth compound (occasionally referred to as a second dopant material).

### Host Material

Herein, the “host material” refers to, for instance, a material that accounts for “50 mass % or more of the layer.” The first emitting layer thus contains, for instance, 50 mass % or more of the first compound with respect to a total mass of the first emitting layer. The second emitting layer contains, for instance, 50 mass % or more of the second compound with respect to a total mass of the second emitting layer. Further, for instance, the “host material” may account for 60 mass % or more of the layer, 70 mass % or more of the layer, 80 mass % or more of the layer, 90 mass % or more of the layer, or 95 mass % or more of the layer.

The compound having at least one deuterium atom is preferably at least one of a host material or a dopant material. In an exemplary embodiment, the compound having at least one deuterium atom is a host material. In an exemplary embodiment, the compound having at least one deuterium atom is a host material, and a dopant material has no deuterium atom.

In an exemplary embodiment, when deuterium atoms in the first and second compounds are substituted with protium atoms, chemical structures of the first and the second compounds are mutually the same or different, preferably mutually different.

In an exemplary embodiment, dopant materials in the first and second emitting layers are mutually the same or different, preferably mutually the same.

The compound having at least one deuterium atom preferably has 1 to 100 deuterium atoms, more preferably has 1 to 80 deuterium atoms.

The compound having at least one deuterium atom as a dopant material preferably has 1 to 100 deuterium atoms, and more preferably has 1 to 80 deuterium atoms.

The compound having at least one deuterium atom as a host material preferably has 1 to 50 deuterium atoms, and more preferably has 1 to 40 deuterium atoms.

In an exemplary embodiment, at least one of the first emitting layer or the second emitting layer contains one or more of host materials.

The emitting layer containing two or more of host materials may contain a host material having a deuterium atom. In that case, only one of the host materials may be a compound having a deuterium atom and the other may be a compound having no deuterium atom, or both of the host materials may each be a compound having a deuterium atom.

### Singlet Energy S₁

A method of measuring a singlet energy S₁ with use of a solution (occasionally referred to as a solution method) is exemplified by a method below.

A toluene solution of a measurement target compound at a concentration ranging from 10⁻⁵ mol/L to 10⁻⁴ mol/L is prepared and put in a quartz cell. An absorption spectrum (ordinate axis: absorption intensity, abscissa axis: wavelength) of the thus-obtained sample is measured at a normal temperature (300K). A tangent is drawn to the fall of the absorption spectrum close to the long-wavelength region, and a wavelength value λedge (nm) at an intersection of the tangent and the abscissa axis is assigned to a conversion equation (F2) below to calculate the singlet energy.

S₁ [eV]=1239.85/λedge Conversion Equation (F2):

Any device for measuring absorption spectrum is usable. For instance, a spectrophotometer (U3310 manufactured by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

In the organic electroluminescence device according to the exemplary embodiment, a triplet energy T₁(M1) of the first compound is preferably different from a triplet energy T₁(M2) of the second compound.

### Triplet Energy T₁

A method of measuring triplet energy T₁ is exemplified by a method below.

A measurement target compound is dissolved in EPA (diethylether:isopentane:ethanol=5:5:2 in volume ratio) so as to fall within a range from 10⁻⁵ mol/L to 10⁻⁴ mol/L, and the obtained solution is put in a quartz cell to provide a measurement sample. A phosphorescence spectrum (ordinate axis: phosphorescent luminous intensity, abscissa axis: wavelength) of the measurement sample is measured at a low temperature (77K). A tangent is drawn to the rise of the phosphorescence spectrum close to the short-wavelength region. An energy amount is calculated by a conversion equation (F1) below on a basis of a wavelength value λ_{edge} [nm] at an intersection of the tangent and the abscissa axis. The calculated energy amount is defined as triplet energy T₁.

T₁ [eV]=1239.85/λ_{edge} Conversion Equation (F1):

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 (manufactured by Hitachi High-Technologies Corporation) is usable. Any device for phosphorescence measurement is usable. A combination of a cooling unit, a low temperature container, an excitation light source and a light-receiving unit may be used for phosphorescence measurement.

The first emitting layer and the second emitting layer preferably do not contain a phosphorescent material (dopant material).

The first emitting layer and the second emitting layer preferably do not contain a heavy metal complex and a phosphorescent rare earth metal complex. Examples of the heavy-metal complex herein include iridium complex, osmium complex, and platinum complex.

In the organic electroluminescence device according to the exemplary embodiment, the first emitting layer also preferably contains no metal complex.

In the organic electroluminescence device according to the exemplary embodiment, the second emitting layer also preferably contains no metal complex.

Both of the first and second emitting layers also preferably contain no metal complex.

### Film Thickness of Emitting Layer

The film thickness of the emitting layer of the organic EL device according to the exemplary embodiment is preferably in a range from 5 nm to 50 nm, more preferably in a range from 7 nm to 50 nm, and further preferably in a range from 10 nm to 50 nm. When the film thickness of the emitting layer is 5 nm or more, the emitting layer is easily formable and chromaticity is easily adjustable. When the film thickness of the emitting layer is 50 nm or less, a rise in the drive voltage is easily reducible. The film thickness of first and second emitting layers may be mutually the same or different.

### Content Ratios of Compounds in Emitting Layer

When the first emitting layer contains the first compound and the third compound, a content ratio of each of the first compound and the third compound in the first emitting layer preferably falls, for instance, within a range below.

The content ratio of the first compound is preferably in a range from 80 mass % to 99 mass %, more preferably in a range from 90 mass % to 99 mass %, and further preferably in a range from 95 mass % to 99 mass %.

The content ratio of the third compound is preferably in a range from 1 mass % to 10 mass %, more preferably in a range from 1 mass % to 7 mass %, and further preferably in a range from 1 mass % to 5 mass %.

The upper limit of the total of the content ratios of the first compound and the third compound in the first emitting layer is 100 mass %.

It is not excluded that the first emitting layer of the exemplary embodiment further contains a material(s) other than the first and third compounds.

The first emitting layer may contain a single type of the first compound or may contain two or more types of the first compound. The first emitting layer may contain a single type of the third compound or may contain two or more types of the third compound.

When the second emitting layer contains the second compound and the fourth compound, a content ratio of each of the second compound and the fourth compound in the second emitting layer preferably falls, for instance, within a range below.

The content ratio of the second compound is preferably in a range from 80 mass % to 99 mass %, more preferably in a range from 90 mass % to 99 mass %, and further preferably in a range from 95 mass % to 99 mass %.

The content ratio of the fourth compound is preferably in a range from 1 mass % to 10 mass %, more preferably in a range from 1 mass % to 7 mass %, and further preferably in a range from 1 mass % to 5 mass %.

The upper limit of the total of the content ratios of the second compound and the fourth compound in the second emitting layer is 100 mass %.

It is not excluded that the second emitting layer of the exemplary embodiment further contains a material(s) other than the second and fourth compounds.

The second emitting layer may contain a single type of the second compound or may contain two or more types of the second compound. The second emitting layer may contain a single type of the fourth compound or may contain two or more types of the fourth compound.

### Emission Wavelength of Organic EL Device

The organic electroluminescence device of the exemplary embodiment preferably emits light having a maximum peak wavelength in a range from 430 nm to 480 nm when being driven.

The maximum peak wavelength of the light emitted from the organic EL element when being driven is measured as follows. Voltage is applied on the organic EL device such that a current density is 10 mA/cm², where spectral radiance spectrum is measured by a spectroradiometer CS-2000 (manufactured by Konica Minolta, Inc.). A peak wavelength of an emission spectrum, at which the luminous intensity of the resultant spectral radiance spectrum is at the maximum, is measured and defined as the maximum peak wavelength (unit: nm).

In the organic EL device according to the exemplary embodiment, the second emitting layer is also preferably provided between the first emitting layer and the cathode. Specifically, the first emitting layer and the second emitting layer are also preferably laminated in this order from the anode.

In the organic EL device according to the exemplary embodiment, the second emitting layer is also preferably provided between the first emitting layer and the anode. Specifically, the second emitting layer and the first emitting layer are also preferably laminated in this order from the anode.

When the first emitting layer and the second emitting layer are laminated in this order from the anode in the organic EL device according to the exemplary embodiment, an electron mobility μe(H1) of the first host material and an electron mobility μe(H2) of the second host material satisfy a relationship of a numerical formula (Numerical Formula 30) below.

μe(H2)>μe(H1) (Numerical Formula 30)

When the first host material and the second host material satisfy the relationship of the numerical formula (Numerical Formula 30), a recombination ability between holes and electrons in the first emitting layer is improved.

When the first emitting layer and the second emitting layer are laminated in this order from the anode in the organic EL device according to the exemplary embodiment, a hole mobility μh(H1) of the first host material and a hole mobility μh(H2) of the second host material also preferably satisfy a relationship of a numerical formula (Numerical Formula 31) below.

μh(H1)>μh(H2) (Numerical Formula 31)

When the first emitting layer and the second emitting layer are laminated in this order from the anode in the organic EL device according to the exemplary embodiment, the hole mobility μh(H1) of the first host material, the electron mobility μe(H1) of the first host material, the hole mobility μh(H2) of the second host material, and the electron mobility μe(H2) of the second host material also preferably satisfy a relationship of a numerical formula (Numerical Formula 32) below.

(μe(H2)/μh(H2))>(μe(H1)/μh(H1)) (Numerical Formula 32)

The electron mobility can be measured according to an impedance measurement using a mobility evaluation device manufactured by the following steps. The mobility evaluation device is, for instance, manufactured by the following steps.

A compound Target, which is to be measured for an electron mobility, is vapor-deposited on a glass substrate having an aluminum electrode (anode) so as to cover the aluminum electrode, thereby forming a measurement target layer. A compound ET-A below is vapor-deposited on this measurement target layer to form an electron transporting layer. LiF is vapor-deposited on the formed electron transporting layer to form an electron injecting layer. Metal aluminum (Al) is vapor-deposited on the formed electron injecting layer to form a metal cathode.

An arrangement of the mobility evaluation device above is roughly shown as follows.
glass/Al(50)/Target(200)/ET-A(10)/LiF(1)/Al(50)

Numerals in parentheses represent a film thickness (nm).

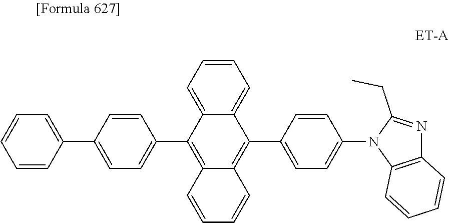 [SMILES: CCc1nc2ccccc2n1-c1ccc(-c2c3ccccc3c(-c3ccc(-c4ccccc4)cc3)c3ccccc23)cc1]

The mobility evaluation device for an electron mobility is set in an impedance measurement device to perform an impedance measurement. In the impedance measurement, a measurement frequency is swept from 1 Hz to 1 MHz. At this time, an alternating current amplitude of 0.1 V and a direct current voltage V are applied to the device. A modulus M is calculated from a measured impedance Z using a relationship of a calculation formula (C1) below.

M=jωZ Calculation Formula (C1):

In the calculation formula (C1), j is an imaginary unit whose square is −1 and ω is an angular frequency [rad/s].

In a bode plot in which an imaginary part of the modulus M is represented by an ordinate axis and the frequency [Hz] is represented by an abscissa axis, an electrical time constant T of the mobility evaluation device is obtained from a frequency fmax showing a peak using a calculation formula (C2) below.

τ=1/(2πf max) Calculation Formula (C2):

π in the calculation formula (C2) is a symbol representing a circumference ratio.

An electron mobility μe is calculated from a relationship of a calculation formula (C3-1) below using τ.

μe=d²/(V_{T}) Calculation Formula (C3-1):
d in the calculation formula (C3-1) is a total film thickness of organic thin film(s) forming the device. In a case of the arrangement of the mobility evaluation device for an electron mobility, d=210 [nm] is satisfied.

The hole mobility can be measured according to an impedance measurement using a mobility evaluation device manufactured by the following steps. The mobility evaluation device is, for instance, manufactured by the following steps.

A compound HA-2 below is vapor-deposited on a glass substrate having an ITO transparent electrode (anode) so as to cover the transparent electrode, thereby forming a hole injecting layer. A compound HT-A below is vapor-deposited on the formed hole injecting layer to form a hole transporting layer. Subsequently, a compound Target, which is to be measured for a hole mobility, is vapor-deposited to form a measurement target layer. Metal aluminum (Al) is vapor-deposited on this measurement target layer to form a metal cathode.

An arrangement of the mobility evaluation device above is roughly shown as follows.
ITO(130)/HA-2(5)/HT-A(10)/Target(200)/Al(80)
Numerals in parentheses represent a film thickness (nm).

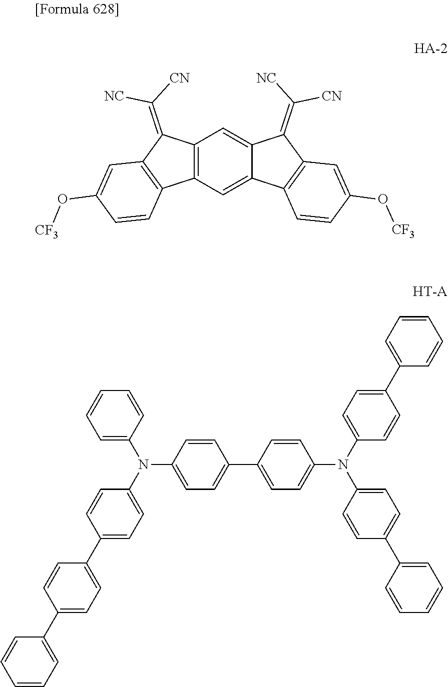 [SMILES: N#CC(C#N)=C1c2cc(OC(F)(F)F)ccc2-c2cc3c(cc21)C(=C(C#N)C#N)c1cc(OC(F)(F)F)ccc1-3 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

The mobility evaluation device for a hole mobility is set in an impedance measurement device to perform an impedance measurement. In the impedance measurement, a measurement frequency is swept from 1 Hz to 1 MHz. At this time, an alternating current amplitude of 0.1 V and a direct current voltage V are applied to the device. A modulus M is calculated from a measured impedance Z using the relationship of the calculation formula (C1).

In a bode plot in which an imaginary part of the modulus M is represented by an ordinate axis and the frequency [Hz] is represented by an abscissa axis, an electrical time constant τ of the mobility evaluation device is obtained from a frequency fmax showing a peak using the calculation formula (C2).

A hole mobility μh is calculated from a relationship of a calculation formula (C₃₋₂) below using T obtained from the calculation formula (C2).

μh=d²/(V_{T}) Calculation Formula (C3-2):
d in the calculation formula (C3-2) is a total film thickness of organic thin film(s) forming the device. In a case of the arrangement of the mobility evaluation device for a hole mobility, d=215 [nm] is satisfied.

The electron mobility and the hole mobility herein are each a value obtained in a case where a square root of an electric field intensity meets E^{1/2}=500 [V^{1/2}/cm^{1/2}]. The square root of the electric field intensity, E^{1/2}, can be calculated from a relationship of a calculation formula (C4) below.

E^{1/2}=V1/²/d^{1/2} Calculation Formula (C4):

For the impedance measurement, a 1260 type by Solartron Analytical is used as the impedance measurement device, and for a higher accuracy, a 1296 type dielectric constant measurement interface by Solartron Analytical can be used together therewith.

The organic EL device according to the exemplary embodiment may include one or more organic layer(s) in addition to the first emitting layer and the second emitting layer. Examples of the organic layer include at least one layer selected from the group consisting of a hole injecting layer, a hole transporting layer, an emitting layer, an electron injecting layer, an electron transporting layer, a hole blocking layer, and an electron blocking layer.

In the organic EL device according to the exemplary embodiment, the organic layer may consist of the first emitting layer and the second emitting layer. Alternatively, the organic layer may further include, for instance, at least one layer selected from the group consisting of a hole injecting layer, a hole transporting layer, an electron injecting layer, an electron transporting layer, a hole blocking layer, and an electron blocking layer.

It is preferable that the organic electroluminescence device of the exemplary embodiment includes a hole transporting layer between the anode and one of the first emitting layer and the second emitting layer provided closer to the anode.

It is preferable that the organic electroluminescence device of the exemplary embodiment includes an electron transporting layer between the cathode and one of the first emitting layer and the second emitting layer provided closer to the cathode.

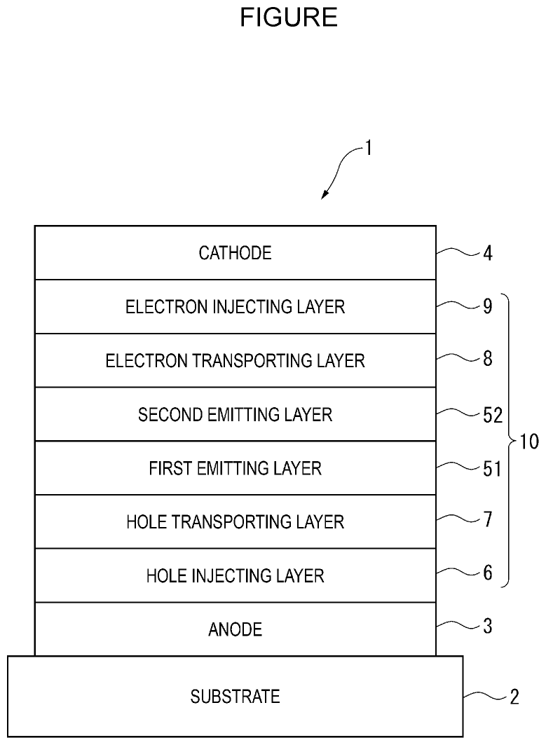

FIG. 1 schematically shows an exemplary arrangement of the organic EL device of the exemplary embodiment.

An organic EL device 1 includes a light-transmissive substrate 2, an anode 3, a cathode 4, and an organic layer 10 provided between the anode 3 and the cathode 4. The organic layer 10 includes a hole injecting layer 6, a hole transporting layer 7, a first emitting layer 51, a second emitting layer 52, an electron transporting layer 8, and an electron injecting layer 9, these of which are laminated in this order from the anode 3.

The invention is not limited to a structure of the organic EL device shown in FIG. 1. In the organic EL device with another arrangement, for instance, the organic layer includes the hole injecting layer, hole transporting layer, second emitting layer, first emitting layer, electron transporting layer, and electron injecting layer, those of which are laminated in this order from the anode.

In the organic EL device of the exemplary embodiment, the first emitting layer and the second emitting layer are preferably in direct contact with each other.

Herein, a layer arrangement in which the first emitting layer and the second emitting layer are in direct contact with each other may include one of embodiments (LS1), (LS2) and (LS3) below.
(LS1) An embodiment in which a region containing both the first host material and the second host material is generated in a process of vapor-depositing the compound of the first emitting layer and vapor-depositing the compound of the second emitting layer, the region being present on the interface between the first emitting layer and the second emitting layer.
(LS2) An embodiment in which in a case of containing an emitting compound in the first emitting layer and the second emitting layer, a region containing the first host material, the second host material and the emitting compound is generated in a process of vapor-depositing the compound of the first emitting layer and vapor-depositing the compound of the second emitting layer, and the region being present on the interface between the first emitting layer and the second emitting layer.
(LS3) An embodiment in which in a case of containing an emitting compound in the first emitting layer and the second emitting layer, a region containing the emitting compound, a region containing the first host material or a region containing the second host material is generated in a process of vapor-depositing the compound of the first emitting layer and vapor-depositing the compound of the second emitting layer, and the region being present on the interface between the first emitting layer and the second emitting layer.

When the first emitting layer and the second emitting layer are not in direct contact with each other in the organic EL device of the exemplary embodiment, at least one organic layer may be provided between the first emitting layer and the second emitting layer.

### Interposed Layer

The organic EL device according to the exemplary embodiment may include an interposed layer as an organic layer disposed between the first emitting layer and the second emitting layer.

In the exemplary embodiment, in order to inhibit an overlap between a Singlet emitting region and a TTF emitting region, the interposed layer contains no emitting compound or may contain an emitting compound in an insubstantial amount provided that the overlap can be inhibited.

For instance, the interposed layer contains 0 mass % of an emitting compound. Alternatively, for instance, the interposed layer may contain an emitting compound provided that the emitting compound contained is a component accidentally mixed in a manufacturing process or a component contained as impurities in a material.

For instance, when the interposed layer consists of a material A, a material B, and a material C, the content ratios of the materials A, B, and C in the interposed layer are each 10 mass % or more, and the total of the content ratios of the materials A, B, and C is 100 mass %.

In the following, the interposed layer is occasionally referred to as a “non-doped layer”. A layer containing an emitting compound is occasionally referred to as a “doped layer”.

It is considered that the Singlet emitting region and the TTF emitting region are typically likely to be separated from each other in laminated emitting layers, thus improving luminous efficiency.

In the organic EL device according to the exemplary embodiment, when the interposed layer (non-doped layer) is disposed between the first emitting layer and the second emitting layer in the emitting region, it is expected that a region where the Singlet emitting region and the TTF emitting region overlap with each other is reduced to inhibit a decrease in TTF efficiency caused by collision between triplet excitons and carriers. That is, it is considered that providing the interposed layer (non-doped layer) between the emitting layers contributes to the improvement in the efficiency of TTF emission.

The interposed layer is a non-doped layer.

The interposed layer contains no metal atom. The interposed layer thus contains no metal complex.

The interposed layer contains an interposed layer material. The interposed layer material is not an emitting compound.

The interposed layer material may be any material except for the emitting compound.

Examples of the interposed layer material include: 1) a heterocyclic compound such as an oxadiazole derivative, benzimidazole derivative, or phenanthroline derivative; 2) a fused aromatic compound such as a carbazole derivative, anthracene derivative, phenanthrene derivative, pyrene derivative or chrysene derivative; and 3) an aromatic amine compound such as a triarylamine derivative or a fused polycyclic aromatic amine derivative.

One or both of the first host material and the second host material may be used as the interposed layer material. The interposed layer material may be any material provided that the Singlet emitting region and the TTF emitting region are separated from each other and the Singlet emission and the TTF emission are not hindered.

In the organic EL device according to the exemplary embodiment, the respective content ratios of all the materials forming the interposed layer in the interposed layer are 10 mass % or more.

The interposed layer contains the interposed layer material as a material forming the interposed layer.

The interposed layer preferably contains 60 mass % or more of the interposed layer material, more preferably contains 70 mass % or more of the interposed layer material, further preferably contains 80 mass % or more of the interposed layer material, more further preferably 90 mass % or more of the interposed layer material, and still further more preferably 95 mass % or more of the interposed layer material, with respect to a total mass of the interposed layer.

The interposed layer may contain a single type of the interposed layer material or may contain two or more types of the interposed layer material.

When the interposed layer contains two or more types of the interposed layer material, the upper limit of the total of the content ratios of the two or more types of the interposed layer material is 100 mass %.

It should be noted that the interposed layer of the exemplary embodiment may further contain material(s) other than the interposed layer material.

The interposed layer may be provided in the form of a single layer or a laminate of two or more layers.

As long as the overlap between the Singlet emitting region and the TTF emitting region is inhibited, the film thickness of the interposed layer is not particularly limited but each layer in the interposed layer is preferably in a range from 3 nm to 15 nm, more preferably in a range from 5 nm to 10 nm.

The interposed layer having a film thickness of 3 nm or more easily separates the Singlet emitting region from the emitting region derived from TTF.

The interposed layer having a film thickness of 15 nm or less easily inhibits a phenomenon where the host material of the interposed layer emits light.

An arrangement of an organic EL device will be further described below. It should be noted that the reference numerals will be occasionally omitted below.

### Substrate

The substrate is used as a support for the organic EL device. For instance, glass, quartz, plastics and the like are usable for the substrate. A flexible substrate is also usable. The flexible substrate is a bendable substrate, which is exemplified by a plastic substrate. Examples of the material for the plastic substrate include polycarbonate, polyarylate, polyethersulfone, polypropylene, polyester, polyvinyl fluoride, polyvinyl chloride, polyimide, and polyethylene naphthalate. Moreover, an inorganic vapor deposition film is also usable.

### Anode

Metal, an alloy, an electrically conductive compound, a mixture thereof, or the like having a large work function (specifically, 4.0 eV or more) is preferably used as the anode formed on the substrate. Specific examples of the material include ITO (Indium Tin Oxide), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the organic layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), alloys including the rare earth metal are also usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Cathode

It is preferable to use metal, an alloy, an electroconductive compound, a mixture thereof, or the like having a small work function (specifically, 3.8 eV or less) for the cathode. Examples of the material for the cathode include elements belonging to Groups 1 and 2 in the periodic table of the elements, specifically, the alkali metal such as lithium (Li) and cesium (Cs), the alkaline earth metal such as magnesium (Mg), calcium (Ca) and strontium (Sr), alloys (e.g., MgAg and AlLi) including the alkali metal or the alkaline earth metal, the rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method and the like.

### Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the highly hole-injectable substance further include: an aromatic amine compound, which is a low-molecule organic compound, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1); and dipyrazino[2,3-f:20,30-h]quinoxaline-2,3,6,7,10,11-hexacarbonitrile (HAT-CN).

In addition, a high polymer compound (e.g., oligomer, dendrimer and polymer) is usable as the substance exhibiting a high hole injectability. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid) (PAni/PSS) are also usable.

### Hole Transporting Layer

The hole transporting layer is a layer containing a highly hole-transporting substance. An aromatic amine compound, carbazole derivative, anthracene derivative and the like are usable for the hole transporting layer. Specific examples of a material for the hole transporting layer include an aromatic amine compound such as 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(V-s) or more.

For the hole transporting layer, a carbazole derivative such as CBP, 9-[4-(N-carbazolyl)]phenyl-10-phenylanthracene (CzPA), and 9-phenyl-3-[4-(10-phenyl-9-anthryl)phenyl]-9H-carbazole (PCzPA) and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. It should be noted that the layer containing the substance exhibiting a high hole transportability may be not only a single layer but also a laminate of two or more layers formed of the above substance(s).

### Electron Transporting Layer

The electron transporting layer is a layer containing a highly electron-transporting substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinato)aluminum (abbreviation: Almq₃), bis(10-hydroxybenzo[h]quinolinato)beryllium (abbreviation: BeBq₂), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. In the exemplary embodiment, a benzimidazole compound is preferably usable. The above-described substances mostly have an electron mobility of 10⁻⁶ cm²/Vs or more. It should be noted that any substance other than the above substance may be used for the electron transporting layer as long as the substance exhibits a higher electron transportability than the hole transportability. The electron transporting layer may be provided in the form of a single layer or a laminate of two or more layers of the above substance(s).

Specific examples of the compound usable for the electron transporting layer include compounds below. It should however be noted that the invention is not limited to the specific examples of the compound.

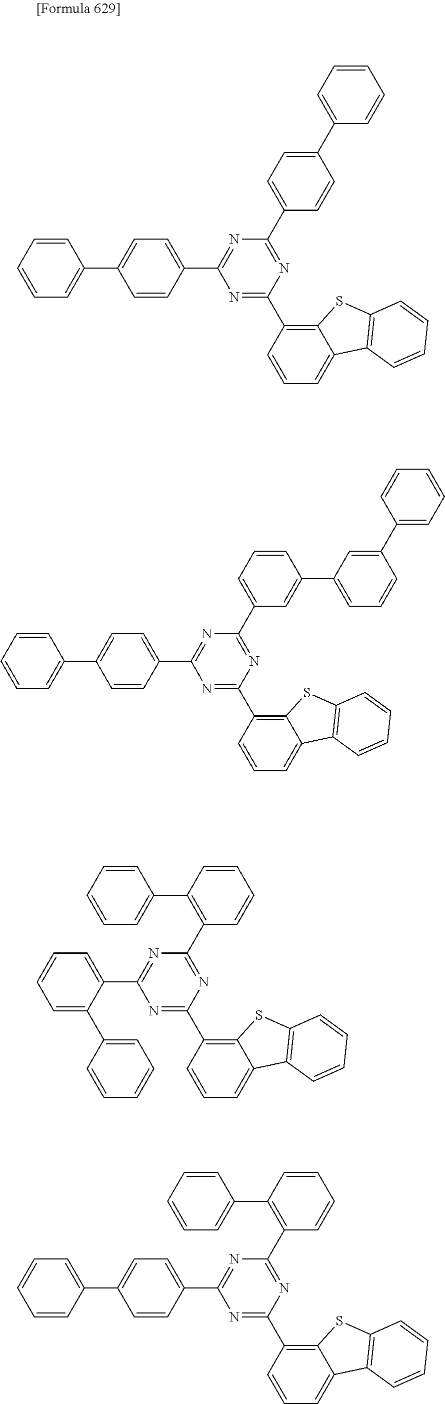 [SMILES: c1ccc(-c2ccc(-c3nc(-c4ccc(-c5ccccc5)cc4)nc(-c4cccc5c4sc4ccccc45)n3)cc2)cc1 ; c1ccc(-c2ccc(-c3nc(-c4cccc(-c5cccc(-c6ccccc6)c5)c4)nc(-c4cccc5c4sc4ccccc45)n3)cc2)cc1 ; c1ccc(-c2ccccc2-c2nc(-c3ccccc3-c3ccccc3)nc(-c3cccc4c3sc3ccccc34)n2)cc1 ; c1ccc(-c2ccc(-c3nc(-c4ccccc4-c4ccccc4)nc(-c4cccc5c4sc4ccccc45)n3)cc2)cc1]

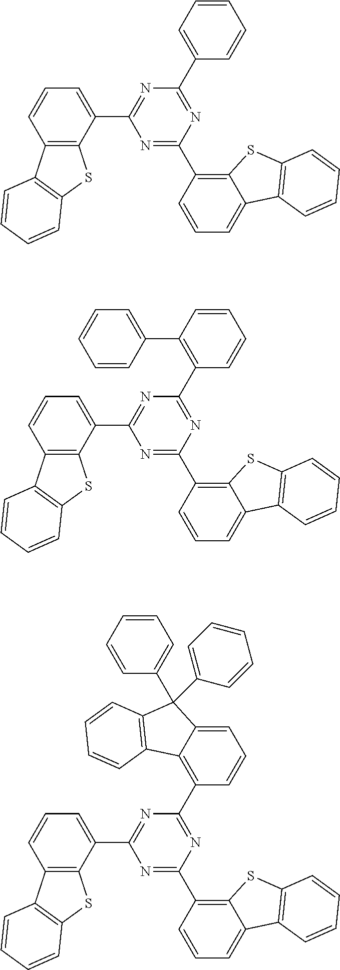 [SMILES: c1ccc(-c2nc(-c3cccc4c3sc3ccccc34)nc(-c3cccc4c3sc3ccccc34)n2)cc1 ; c1ccc(-c2ccccc2-c2nc(-c3cccc4c3sc3ccccc34)nc(-c3cccc4c3sc3ccccc34)n2)cc1 ; c1ccc(C2(c3ccccc3)c3ccccc3-c3c(-c4nc(-c5cccc6c5sc5ccccc56)nc(-c5cccc6c5sc5ccccc56)n4)cccc32)cc1]

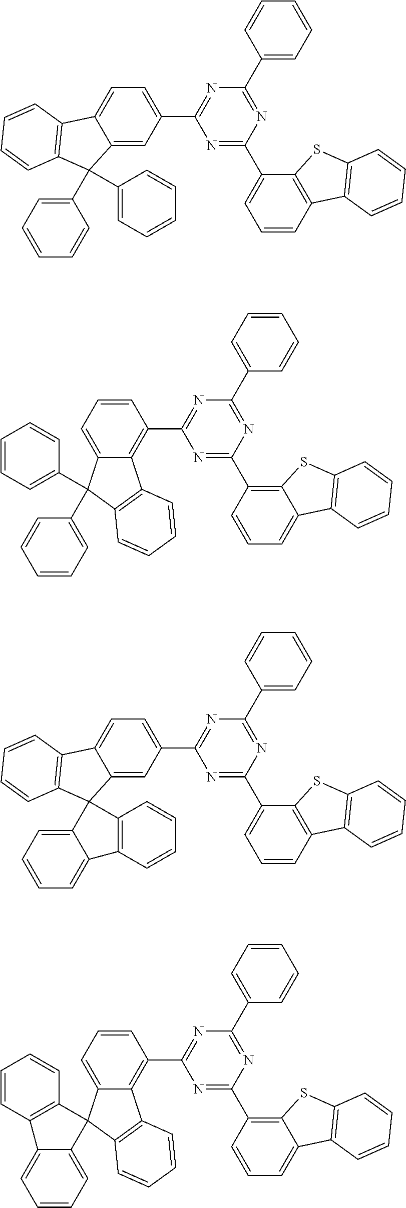 [SMILES: c1ccc(-c2nc(-c3ccc4c(c3)C(c3ccccc3)(c3ccccc3)c3ccccc3-4)nc(-c3cccc4c3sc3ccccc34)n2)cc1 ; c1ccc(-c2nc(-c3cccc4c3-c3ccccc3C4(c3ccccc3)c3ccccc3)nc(-c3cccc4c3sc3ccccc34)n2)cc1 ; c1ccc(-c2nc(-c3ccc4c(c3)C3(c5ccccc5-c5ccccc53)c3ccccc3-4)nc(-c3cccc4c3sc3ccccc34)n2)cc1 ; c1ccc(-c2nc(-c3cccc4c3-c3ccccc3C43c4ccccc4-c4ccccc43)nc(-c3cccc4c3sc3ccccc34)n2)cc1]

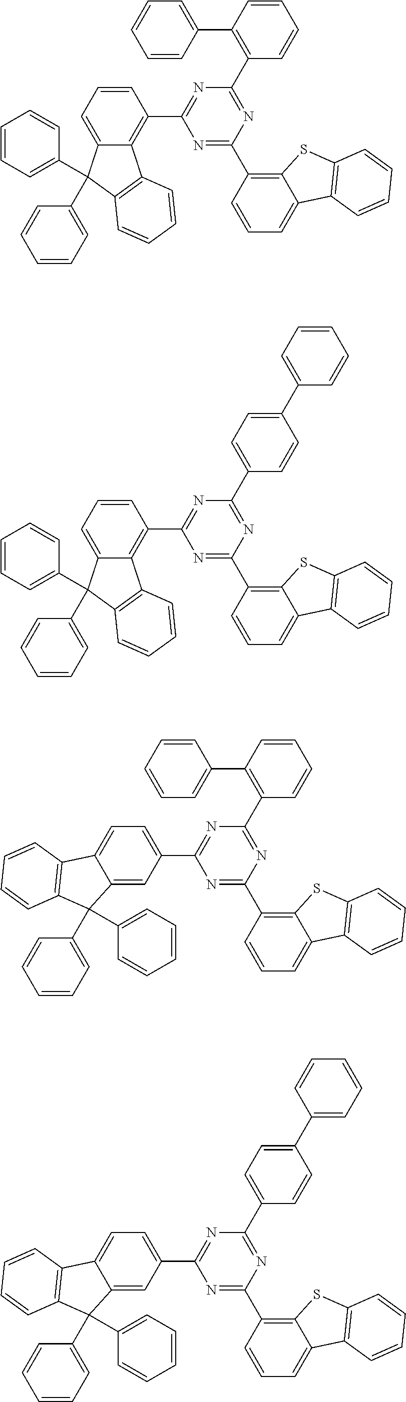 [SMILES: c1ccc(-c2ccccc2-c2nc(-c3cccc4c3-c3ccccc3C4(c3ccccc3)c3ccccc3)nc(-c3cccc4c3sc3ccccc34)n2)cc1 ; c1ccc(-c2ccc(-c3nc(-c4cccc5c4-c4ccccc4C5(c4ccccc4)c4ccccc4)nc(-c4cccc5c4sc4ccccc45)n3)cc2)cc1 ; c1ccc(-c2ccccc2-c2nc(-c3ccc4c(c3)C(c3ccccc3)(c3ccccc3)c3ccccc3-4)nc(-c3cccc4c3sc3ccccc34)n2)cc1 ; c1ccc(-c2ccc(-c3nc(-c4ccc5c(c4)C(c4ccccc4)(c4ccccc4)c4ccccc4-5)nc(-c4cccc5c4sc4ccccc45)n3)cc2)cc1]

Further, a high polymer compound is usable for the electron transporting layer. For instance, poly[(9,9-dihexylfluorene-2,7-diyl)-co-(pyridine-3,5-diyl)](abbreviation: PF-Py), poly[(9,9-dioctylfluorene-2,7-diyl)-co-(2,2′-bipyridine-6,6′-diyl)](abbreviation: PF-BPy) and the like are usable.

### Electron Injecting Layer

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

### Layer Formation Method

A method for forming each layer of the organic EL device in the exemplary embodiment is subject to no limitation except for the above particular description. However, known methods of dry film-forming such as vacuum deposition, sputtering, plasma or ion plating and wet film-forming such as spin coating, dipping, flow coating or ink-jet are applicable.

### Film Thickness

A film thickness of each of the organic layers of the organic EL device in the exemplary embodiment is not limited unless otherwise specified in the above. In general, the thickness preferably ranges from several nanometers to 1 μm because excessively small film thickness is likely to cause defects (e.g. pin holes) and excessively large thickness leads to the necessity of applying high voltage and consequent reduction in efficiency.

In the organic EL device according to the exemplary embodiment, at least one of the first emitting layer that contains the first compound or the second emitting layer that contains the second compound contains a compound having at least one deuterium atom, and at least one of the first emitting layer or the second emitting layer contains a compound having a fused ring that includes four or more rings. Laminating the first emitting layer and the second emitting layer as described above can provide at least one of an organic EL device with enhanced luminous efficiency, an organic EL device emitting light with a long lifetime, or an organic EL device with enhanced luminous efficiency that emits light with a long lifetime.

### Second Exemplary Embodiment

### Electronic Device

An electronic device according to a second exemplary embodiment is installed with any one of the organic EL devices according to the above exemplary embodiment. Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light.

### Modification of Embodiment(s)

The scope of the invention is not limited to the above-described exemplary embodiments but includes any modification and improvement as long as such modification and improvement are compatible with the invention.

For instance, the number of emitting layers is not limited to two, and more than two emitting layers may be provided and laminated with each other. When the organic EL device includes more than two emitting layers, it is only necessary that at least two of the emitting layers should satisfy the requirements mentioned in the above exemplary embodiment. For instance, the rest of the emitting layers may be a fluorescent emitting layer or a phosphorescent emitting layer with use of emission caused by electron transfer from the triplet excited state directly to the ground state.

When the organic EL device includes a plurality of emitting layers, these emitting layers may be mutually adjacently provided, or may form a so-called tandem organic EL device, in which a plurality of emitting units are layered via an intermediate layer.

An arrangement of the organic EL device including three or more emitting layers is as follows.

An organic electroluminescence device includes: an anode; a cathode; a first emitting layer provided between the anode and the cathode and containing a first compound; a second emitting layer provided between the first emitting layer and the cathode and containing a second compound; and a third emitting layer provided between the anode and the cathode and being not in direct contact with any of the first emitting layer and the second emitting layer, in which
at least one of the first emitting layer or the second emitting layer contains a compound having at least one deuterium atom, at least one of the first emitting layer or the second emitting layer contains a compound having a fused ring that includes four or more rings, and the first emitting layer and the second emitting layer are in direct contact with each other.

The third emitting layer also preferably contains the first compound.

The third emitting layer also preferably contains the second compound.

The organic electroluminescence device preferably includes an intermediate layer between the third emitting layer and the first emitting layer and/or an intermediate layer between the third emitting layer and the second emitting layer.

The intermediate layer is generally also referred to as an intermediate electrode, intermediate conductive layer, charge generating layer, electron drawing layer, connection layer, or intermediate insulative layer.

The intermediate layer supplies electrons to a layer that is close to the anode with respect to the intermediate layer, and supplies holes to a layer that is close to the cathode with respect to the intermediate layer. The intermediate layer can be formed from a known material. The intermediate layer may be a single layer, or may be provided by two or more layers. A unit formed by two or more intermediate layers is occasionally referred to as an intermediate unit. The compositions of the plurality of intermediate layers of the intermediate unit are mutually the same or different.

Further, a plurality of layers including the emitting layer that are disposed between the intermediate layer/intermediate unit and the anode/cathode are occasionally referred to as an emitting unit. Examples of the device arrangement of the organic EL device including a plurality of emitting units include (TND1) to (TND4) below.
(TND1) anode/first emitting unit/intermediate layer/second emitting unit/cathode
(TND2) anode/first emitting unit/intermediate unit/second emitting unit/cathode
(TND3) anode/first emitting unit/first intermediate layer/second emitting unit/second intermediate layer/third emitting unit/cathode
(TND4) anode/first emitting unit/first intermediate unit/second emitting unit/second intermediate unit/third emitting unit/cathode

The number of the emitting units and the intermediate layers (or intermediate units) is not limited to the examples shown above.

The first emitting layer and the second emitting layer are preferably included in at least one of the first emitting unit, the second emitting unit, or the third emitting unit.

The first emitting layer and the second emitting layer are also preferably included in all of the emitting units of the organic EL device.

For instance, a blocking layer may be provided adjacent to at least one of a side of the emitting layer close to the anode or a side of the emitting layer close to the cathode. The blocking layer is preferably provided in contact with the emitting layer to block at least any of holes, electrons, or excitons.

For instance, when the blocking layer is provided in contact with the side of the emitting layer close to the cathode, the blocking layer permits transport of electrons and blocks holes from reaching a layer provided closer to the cathode (e.g., the electron transporting layer) beyond the blocking layer. When the organic EL device includes the electron transporting layer, the blocking layer is preferably interposed between the emitting layer and the electron transporting layer.

When the blocking layer is provided in contact with the side of the emitting layer close to the anode, the blocking layer permits transport of holes and blocks electrons from reaching a layer provided closer to the anode (e.g., the hole transporting layer) beyond the blocking layer. When the organic EL device includes the hole transporting layer, the blocking layer is preferably interposed between the emitting layer and the hole transporting layer.

Alternatively, the blocking layer may be provided adjacent to the emitting layer so that excitation energy does not leak out from the emitting layer toward neighboring layer(s). The blocking layer blocks excitons generated in the emitting layer from being transferred to a layer(s) (e.g., the electron transporting layer and the hole transporting layer) closer to the electrode(s) beyond the blocking layer.

The emitting layer is preferably bonded with the blocking layer.

Specific structure, shape and the like of the components in the invention may be designed in any manner as long as an object of the invention can be achieved.

### EXAMPLES

The invention will be described in further detail with reference to Examples. The scope of the invention is by no means limited to Examples.

### Compounds

Structures of compounds used for manufacturing organic EL devices in Examples, Reference Examples, and Comparative Examples are shown below.

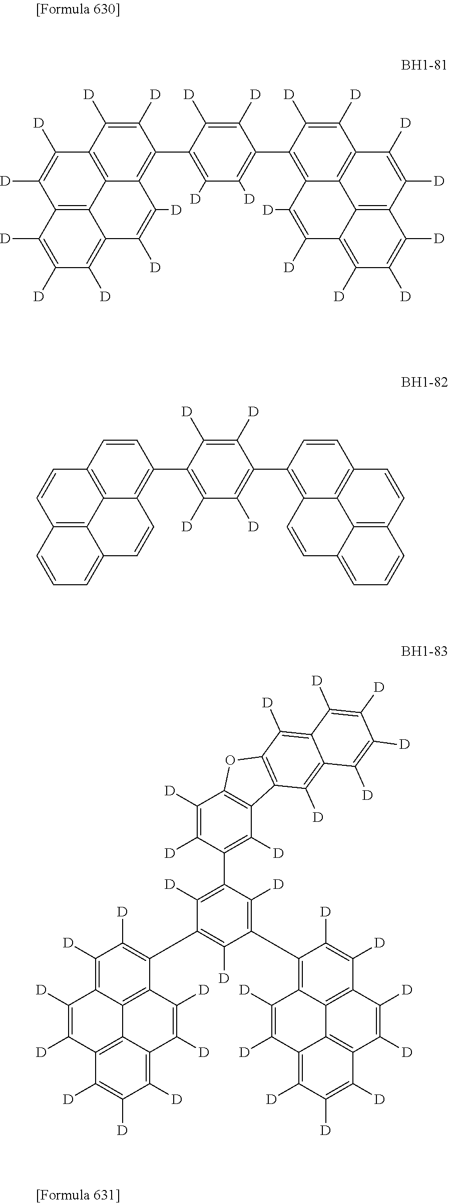

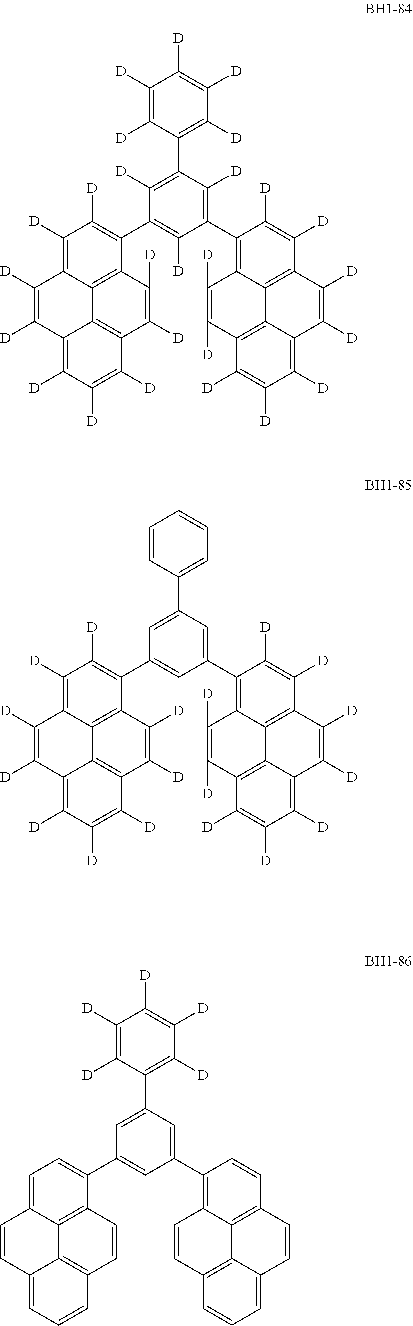

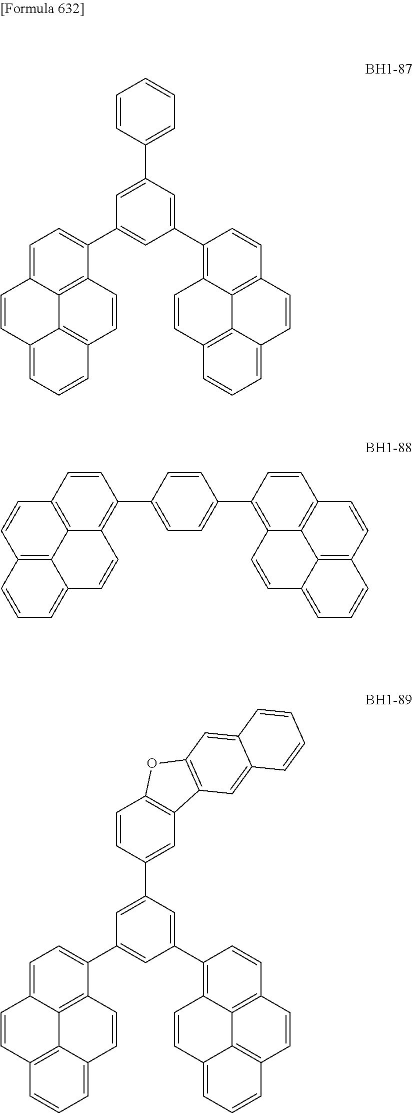

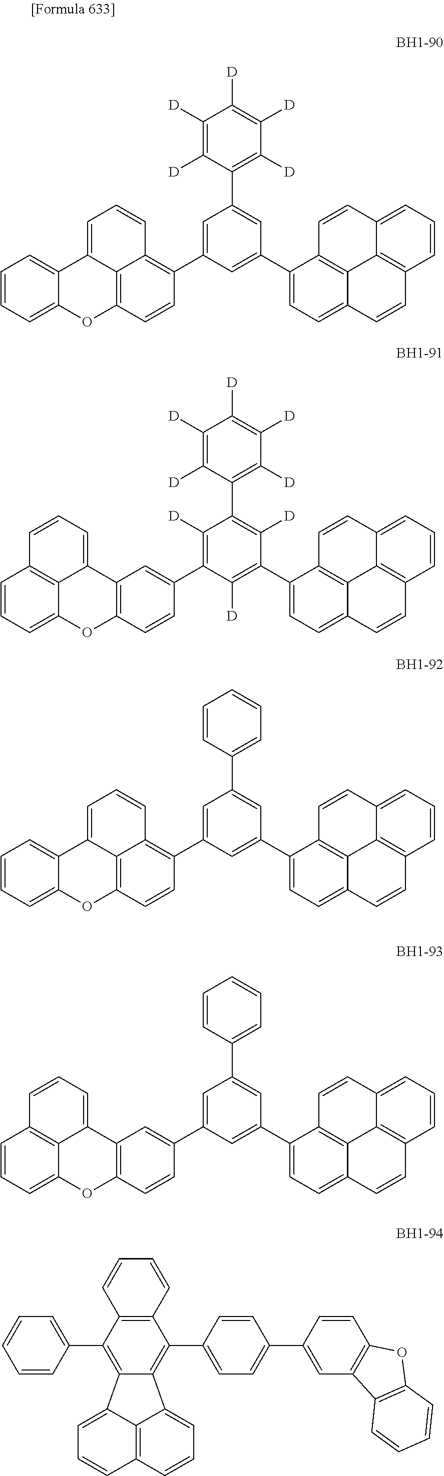 [SMILES: [2H]c1c([2H])c([2H])c(-c2cc(-c3ccc4c5c(cccc35)-c3ccccc3O4)cc(-c3ccc4ccc5cccc6ccc3c4c56)c2)c([2H])c1[2H] ; [2H]c1c([2H])c([2H])c(-c2c([2H])c(-c3ccc4c(c3)-c3cccc5cccc(c35)O4)c([2H])c(-c3ccc4ccc5cccc6ccc3c4c56)c2[2H])c([2H])c1[2H] ; c1ccc(-c2cc(-c3ccc4c5c(cccc35)-c3ccccc3O4)cc(-c3ccc4ccc5cccc6ccc3c4c56)c2)cc1 ; c1ccc(-c2cc(-c3ccc4c(c3)-c3cccc5cccc(c35)O4)cc(-c3ccc4ccc5cccc6ccc3c4c56)c2)cc1 ; c1ccc(-c2c3c(c(-c4ccc(-c5ccc6oc7ccccc7c6c5)cc4)c4ccccc24)-c2cccc4cccc-3c24)cc1]

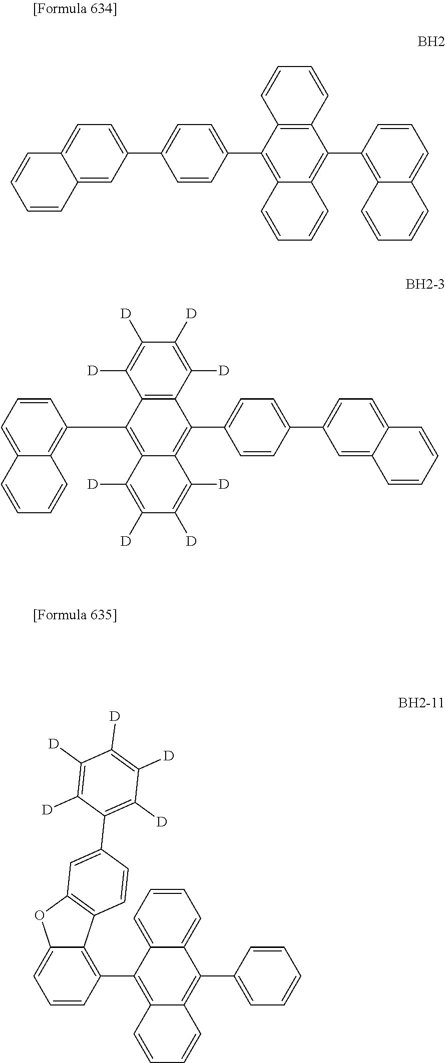

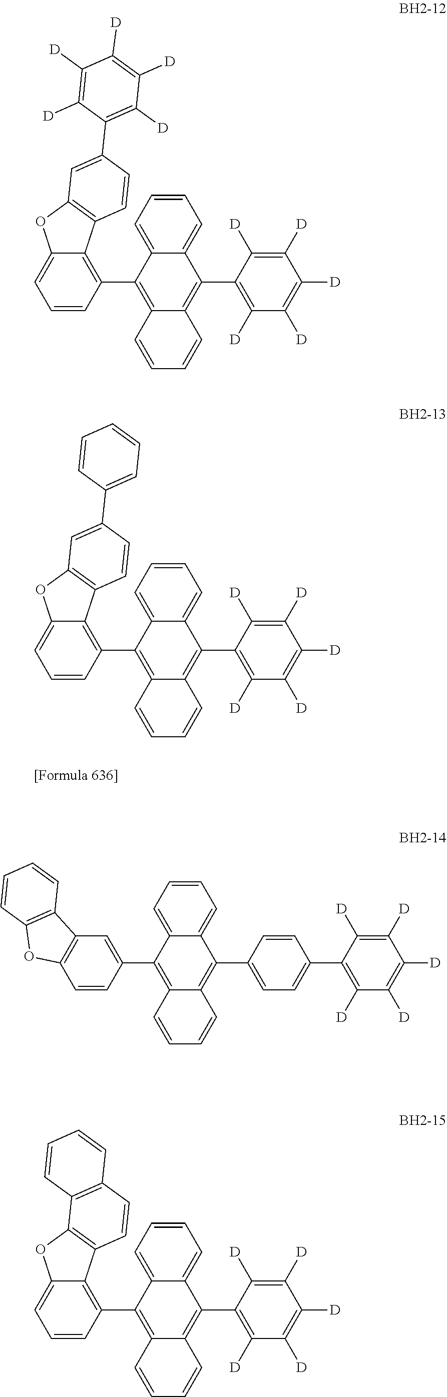 [SMILES: [2H]c1c([2H])c([2H])c(-c2ccc3c(c2)oc2cccc(-c4c5ccccc5c(-c5c([2H])c([2H])c([2H])c([2H])c5[2H])c5ccccc45)c23)c([2H])c1[2H] ; [2H]c1c([2H])c([2H])c(-c2c3ccccc3c(-c3cccc4oc5cc(-c6ccccc6)ccc5c34)c3ccccc23)c([2H])c1[2H] ; [2H]c1c([2H])c([2H])c(-c2ccc(-c3c4ccccc4c(-c4ccc5oc6ccccc6c5c4)c4ccccc34)cc2)c([2H])c1[2H] ; [2H]c1c([2H])c([2H])c(-c2c3ccccc3c(-c3cccc4oc5c6ccccc6ccc5c34)c3ccccc23)c([2H])c1[2H]]

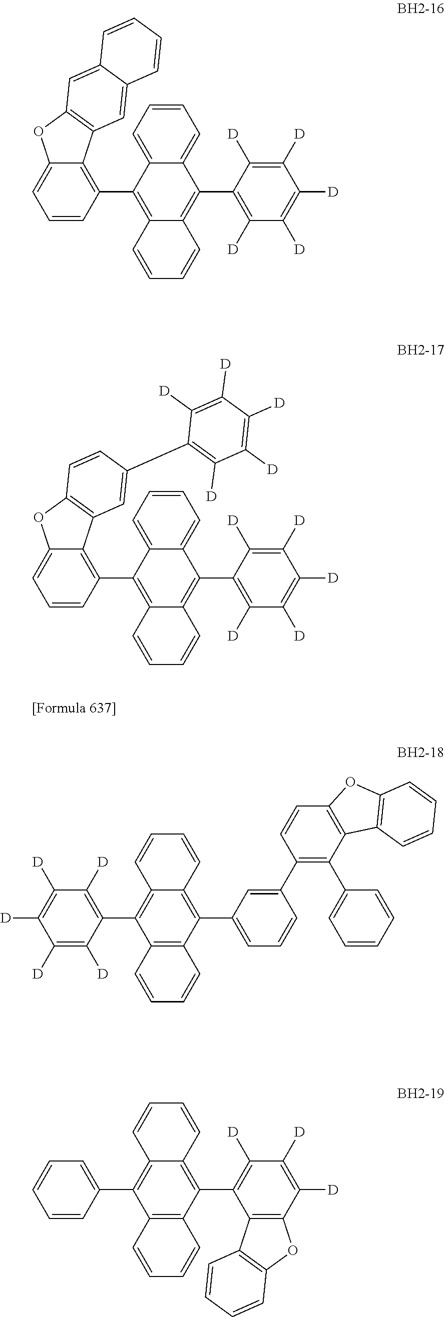 [SMILES: [2H]c1c([2H])c([2H])c(-c2c3ccccc3c(-c3cccc4oc5cc6ccccc6cc5c34)c3ccccc23)c([2H])c1[2H] ; [2H]c1c([2H])c([2H])c(-c2ccc3oc4cccc(-c5c6ccccc6c(-c6c([2H])c([2H])c([2H])c([2H])c6[2H])c6ccccc56)c4c3c2)c([2H])c1[2H] ; [2H]c1c([2H])c([2H])c(-c2c3ccccc3c(-c3cccc(-c4ccc5oc6ccccc6c5c4-c4ccccc4)c3)c3ccccc23)c([2H])c1[2H] ; [2H]c1c([2H])c(-c2c3ccccc3c(-c3ccccc3)c3ccccc23)c2c(oc3ccccc32)c1[2H]]

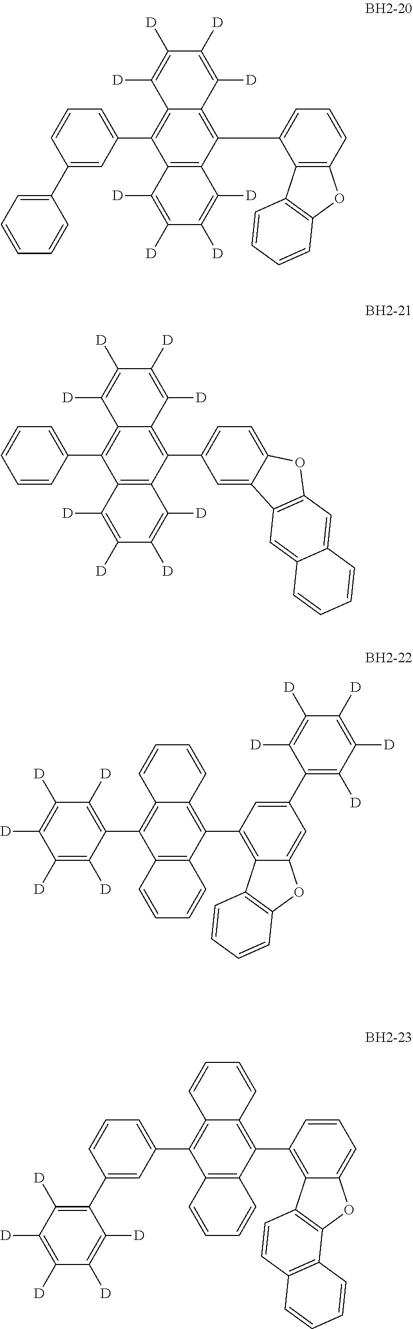 [SMILES: [2H]c1c([2H])c([2H])c2c(-c3cccc4oc5ccccc5c34)c3c([2H])c([2H])c([2H])c([2H])c3c(-c3cccc(-c4ccccc4)c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccccc3)c2c1[2H] ; [2H]c1c([2H])c([2H])c(-c2cc(-c3c4ccccc4c(-c4c([2H])c([2H])c([2H])c([2H])c4[2H])c4ccccc34)c3c(c2)oc2ccccc23)c([2H])c1[2H] ; [2H]c1c([2H])c([2H])c(-c2cccc(-c3c4ccccc4c(-c4cccc5oc6c7ccccc7ccc6c45)c4ccccc34)c2)c([2H])c1[2H]]

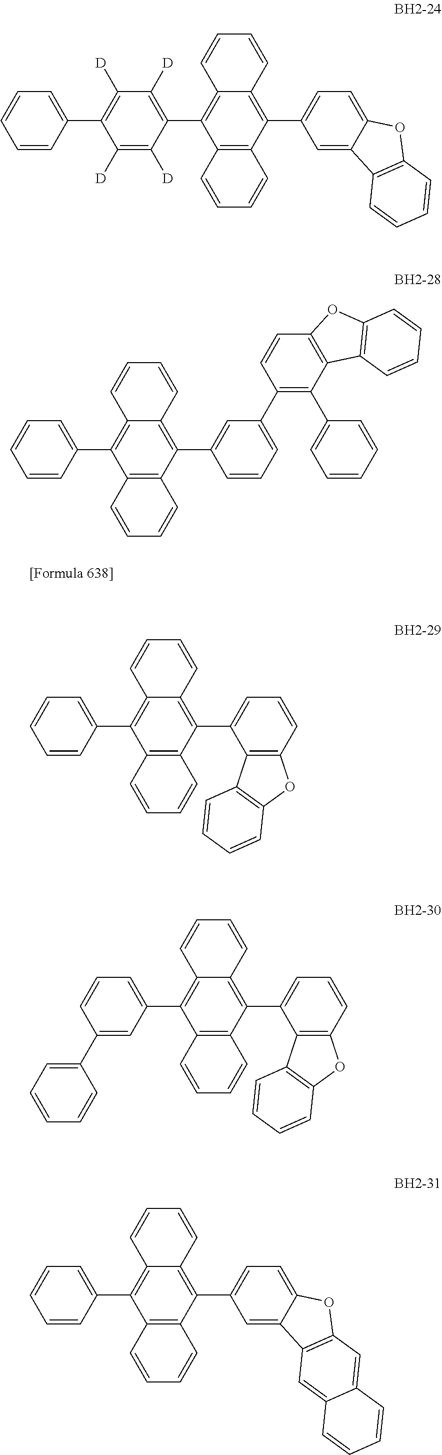 [SMILES: [2H]c1c([2H])c(-c2c3ccccc3c(-c3ccc4oc5ccccc5c4c3)c3ccccc23)c([2H])c([2H])c1-c1ccccc1 ; c1ccc(-c2c3ccccc3c(-c3cccc(-c4ccc5oc6ccccc6c5c4-c4ccccc4)c3)c3ccccc23)cc1 ; c1ccc(-c2c3ccccc3c(-c3cccc4oc5ccccc5c34)c3ccccc23)cc1 ; c1ccc(-c2cccc(-c3c4ccccc4c(-c4cccc5oc6ccccc6c45)c4ccccc34)c2)cc1 ; c1ccc(-c2c3ccccc3c(-c3ccc4oc5cc6ccccc6cc5c4c3)c3ccccc23)cc1]

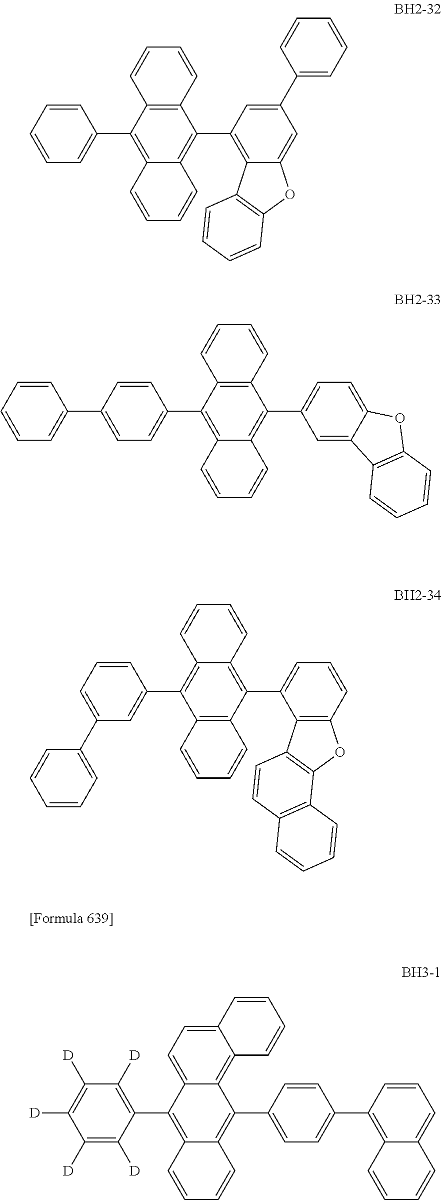 [SMILES: c1ccc(-c2cc(-c3c4ccccc4c(-c4ccccc4)c4ccccc34)c3c(c2)oc2ccccc23)cc1 ; c1ccc(-c2ccc(-c3c4ccccc4c(-c4ccc5oc6ccccc6c5c4)c4ccccc34)cc2)cc1 ; c1ccc(-c2cccc(-c3c4ccccc4c(-c4cccc5oc6c7ccccc7ccc6c45)c4ccccc34)c2)cc1 ; [2H]c1c([2H])c([2H])c(-c2c3ccccc3c(-c3ccc(-c4cccc5ccccc45)cc3)c3c2ccc2ccccc23)c([2H])c1[2H]]

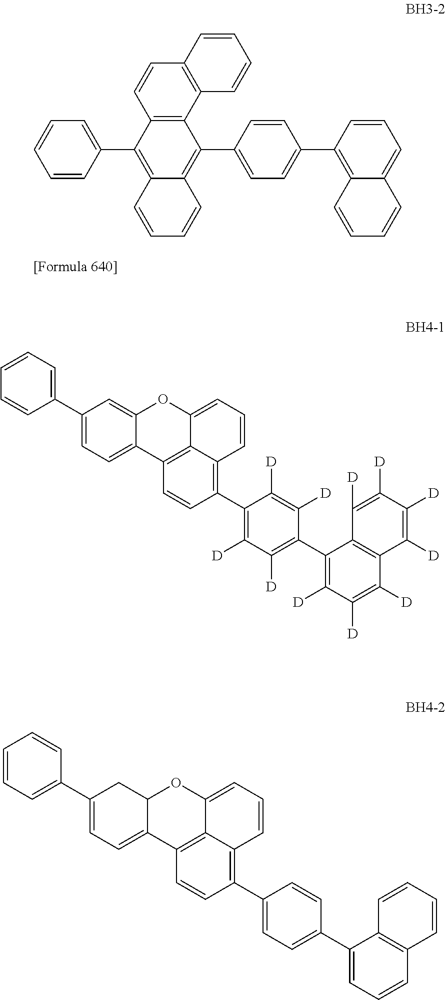

Structures of other compounds used for manufacturing organic EL devices in Examples, Reference Examples and Comparative Examples are shown below.

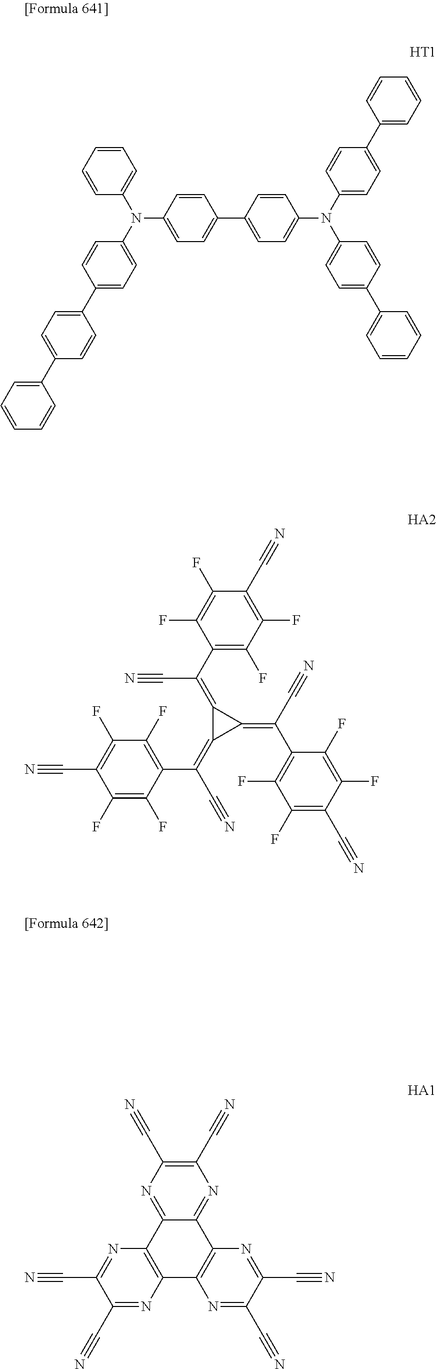 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; N#Cc1nc2c3nc(C#N)c(C#N)nc3c3nc(C#N)c(C#N)nc3c2nc1C#N]

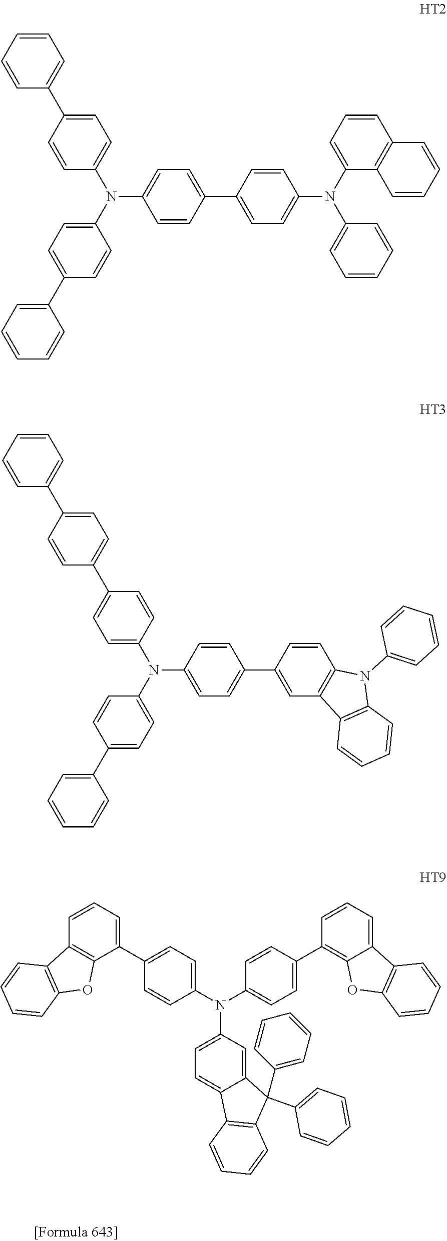 [SMILES: c1ccc(-c2ccc(N(c3ccc(-c4ccccc4)cc3)c3ccc(-c4ccc(N(c5ccccc5)c5cccc6ccccc56)cc4)cc3)cc2)cc1 ; c1ccc(-c2ccc(-c3ccc(N(c4ccc(-c5ccccc5)cc4)c4ccc(-c5ccc6c(c5)c5ccccc5n6-c5ccccc5)cc4)cc3)cc2)cc1 ; c1ccc(C2(c3ccccc3)c3ccccc3-c3ccc(N(c4ccc(-c5cccc6c5oc5ccccc56)cc4)c4ccc(-c5cccc6c5oc5ccccc56)cc4)cc32)cc1]

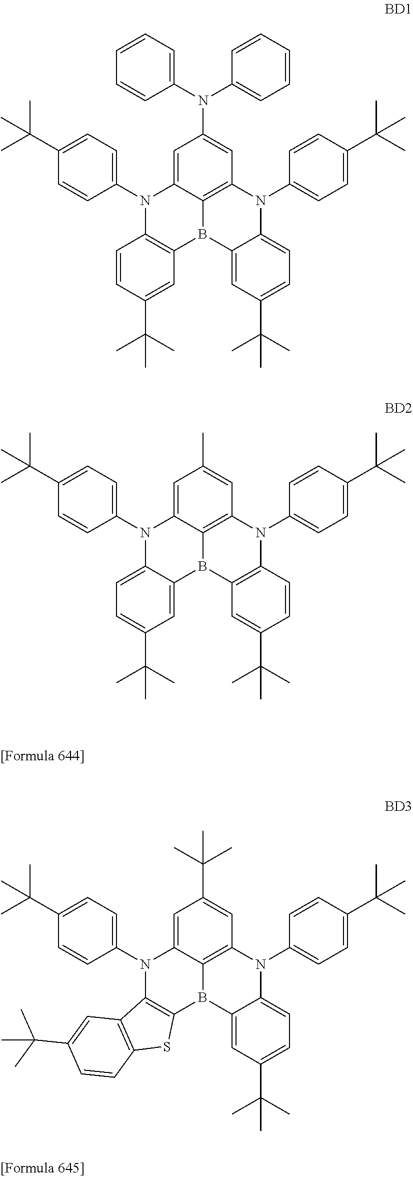

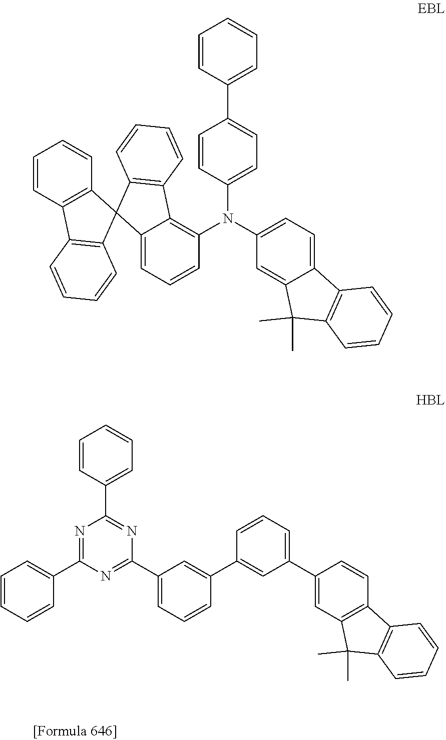 [SMILES: CC1(C)c2ccccc2-c2ccc(N(c3ccc(-c4ccccc4)cc3)c3cccc4c3-c3ccccc3C43c4ccccc4-c4ccccc43)cc21 ; CC1(C)c2ccccc2-c2ccc(-c3cccc(-c4cccc(-c5nc(-c6ccccc6)nc(-c6ccccc6)n5)c4)c3)cc21]

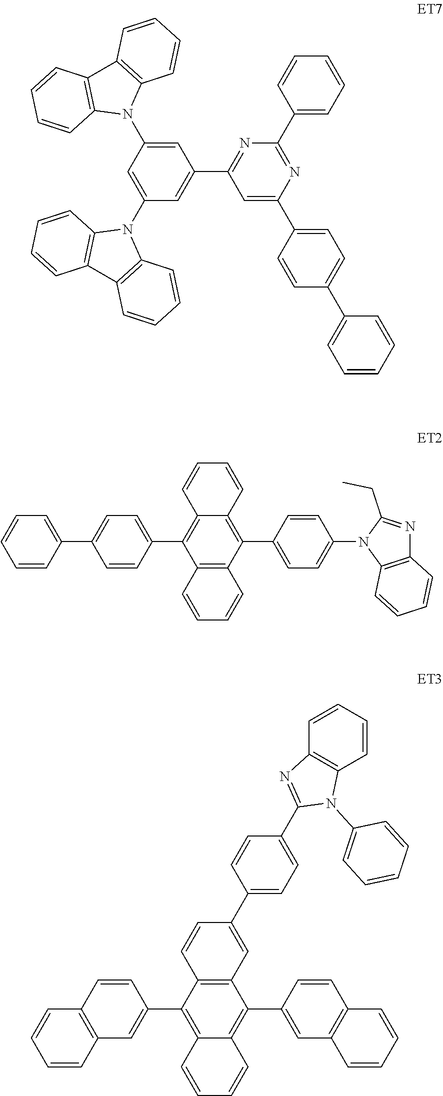 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; CCc1nc2ccccc2n1-c1ccc(-c2c3ccccc3c(-c3ccc(-c4ccccc4)cc3)c3ccccc23)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

### Preparation of Organic EL Device

Organic EL devices were manufactured and evaluated as follows.

### Example 1

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. A film thickness of the ITO transparent electrode was 130 nm.

The cleaned glass substrate having the transparent electrode line was attached to a substrate holder of a vacuum deposition apparatus. Initially, a compound HT1 and the compound HA2 were co-deposited on a surface provided with the transparent electrode line to cover the transparent electrode, thereby forming a 5-nm-thick hole injecting layer (HI). The ratios of the compound HT1 and the compound HA2 in the hole injecting layer were 97 mass % and 3 mass %, respectively.

After the formation of the hole injecting layer, the compound HT1 was vapor-deposited to form an 80-nm-thick first hole transporting layer (HT).

After the formation of the first hole transporting layer, a compound HT9 was vapor-deposited to form a 10-nm-thick second hole transporting layer (also referred to as an electron blocking layer (EBL)).

A compound BH1-84 (first host material (BH)) and a compound BD2 (dopant material (BD)) were co-deposited on the second hole transporting layer such that the ratio of the compound BD2 accounted for 2 mass %, thereby forming a 5-nm-thick first emitting layer.

A compound BH2-3 (second host material (BH)) and the compound BD2 (dopant material (BD)) were co-deposited on the first emitting layer such that the ratio of the compound BD2 accounted for 2 mass %, thereby forming a 20-nm-thick second emitting layer.

A compound ET7 was vapor-deposited on the second emitting layer to form a 10-nm-thick first electron transporting layer (also referred to as a hole blocking layer (HBL)).

A compound ET2 was vapor-deposited on the first electron transporting layer to form a 15-nm-thick second electron transporting layer (ET).

LiF was vapor-deposited on the second electron transporting layer to form a 1-nm-thick electron injecting layer.

Metal (Al) was vapor-deposited on the electron injecting layer to form an 80-nm-thick cathode.

A device arrangement of the organic EL device in Example 1 is roughly shown as follows.

### ITO(130)/HT1: HA2(5,97%:3%)/HT1(80)/HT9(10)/BH1-84:BD2(5,98%:2%)/BH2-3:BD2(20.98%:2%)/ET7(10)/ET2(15)/LiF(1)/Al(80)

Numerals in parentheses represent a film thickness (unit: nm).

The numerals (97%:3%) represented by percentage in the same parentheses indicate a ratio (mass %) between the compound HT1 and the compound HA2 in the hole injecting layer, and the numerals (98%:2%) represented by percentage in the same parentheses indicate a ratio (mass %) between the host material (the compound BH1-84 or the compound BH2-3) and the compound BD2 in the first emitting layer or the second emitting layer. Similar notations apply to the description below.

### Examples 2 to 4

The organic EL devices of Examples 2 and 3 were each manufactured in the same manner as that of Example 1 except that the compound BH1-84 (first host material) in the first emitting layer was replaced with the first compound listed in Table 2.

The organic EL device of Example 4 was manufactured in the same manner as that of Example 1 except that the compound BH2-3 (second host material) in the second emitting layer was replaced with the second compound listed in Table 2.

### Reference Example 1

The organic EL device of Reference Example 1 was manufactured in the same manner as that of Example 4 except that the compound BH1-84 (first host material) in the first emitting layer was replaced with the first compound listed in Table 2.

### Reference Examples 2 to 4

As shown in Table 2, the organic EL devices of Reference Examples 2 to 4 were each manufactured in the same manner as that of Example 1 except that a 25-nm-thick first emitting layer was formed as the emitting layer, the first electron transporting layer was formed on the first emitting layer without forming the second emitting layer, and the compound BH1-84 (first host material) in the first emitting layer was replaced with the first compound listed in Table 2.

### Comparative Example 1

As shown in Table 2, the organic EL device of Comparative Example 1 was manufactured in the same manner as that of Example 1 except that a 25-nm-thick second emitting layer was formed as the emitting layer on the second hole transporting layer without forming the first emitting layer.

### Comparative Example 2

As shown in Table 2, the organic EL device of Comparative Example 2 was manufactured in the same manner as that of Reference Example 1 except that a 25-nm-thick first emitting layer was formed as the emitting layer, and the first electron transporting layer was formed on the first emitting layer without forming the second emitting layer.

### Comparative Example 3

As shown in Table 2, the organic EL device of Comparative Example 3 was manufactured in the same manner as that of Reference Example 1 except that a 25-nm-thick second emitting layer was formed as the emitting layer on the second hole transporting layer without forming the first emitting layer.

### Evaluation of Organic EL Devices

The organic EL devices manufactured were evaluated as follows. Tables 2 to 17 show the evaluation results. In Tables, the first compound corresponds to the first host material and the second compound corresponds to the second host material.

### External Quantum Efficiency EQE

Voltage was applied on the organic EL devices so that a current density was 10 mA/cm², where spectral radiance spectrum was measured by a spectroradiometer (CS-2000 manufactured by Konica Minolta, Inc.). The external quantum efficiency EQE (unit: %) was calculated based on the obtained spectral-radiance spectra, assuming that the spectra was provided under a Lambertian radiation.

### Lifetime LT95

Voltage was applied on the resultant organic EL devices so that a current density was 50 mA/cm², where a time (LT95 (unit: hr)) elapsed before a luminance intensity was reduced to 95% of the initial luminance intensity was measured.

TABLE 2

| | First emitting layer | | | Second emitting layer | | | | |
|---|---|---|---|---|---|---|---|---|
| | | | Film | | | Film | | |
| | First | Third | thickness | Second | Fourth | thickness | EQE | LT95 |
| | compound | compound | [nm] | compound | compound | [nm] | [%] | [hr] |
| Ex. 1 | BH1-84 | BD2 | 5 | BH2-3 | BD2 | 20 | 9.6 | 320 |
| Ex. 2 | BH1-85 | BD2 | 5 | BH2-3 | BD2 | 20 | 9.6 | 250 |
| Ex. 3 | BH1-86 | BD2 | 5 | BH2-3 | BD2 | 20 | 9.6 | 160 |
| Ex. 4 | BH1-84 | BD2 | 5 | BH2 | BD2 | 20 | 9.7 | 255 |
| Ref. Ex. 1 | BH1-87 | BD2 | 5 | BH2 | BD2 | 20 | 9.8 | 150 |
| Ref. Ex. 2 | BH1-84 | BD2 | 25 | — | — | — | 7.2 | 80 |
| Ref. Ex. 3 | BH1-85 | BD2 | 25 | — | — | — | 7.2 | 75 |
| Ref. Ex. 4 | BH1-86 | BD2 | 25 | — | — | — | 7.2 | 75 |
| Comp. 1 | — | — | — | BH2-3 | BD2 | 25 | 8.5 | 145 |
| Comp. 2 | BH1-87 | BD2 | 25 | — | — | — | 7.3 | 75 |
| Comp. 3 | — | — | — | BH2 | BD2 | 25 | 8.8 | 78 |

### Examples 5 to 8

The organic EL devices of Examples 5 to 8 were each manufactured in the same manner as that of Example 1 except that at least one of the compound BH1-84 (first host material) in the first emitting layer or the compound BH2-3 in the second emitting layer was replaced with the compound listed in Table 3.

### Reference Examples 5 and 6

The organic EL devices of Reference Examples 5 and 6 were each manufactured in the same manner as that of Example 1 except that at least one of the compound BH1-84 (first host material) in the first emitting layer or the compound BH2-3 in the second emitting layer was replaced with the compound listed in Table 3.

### Reference Example 7

As shown in Table 3, the organic EL device of Reference Example 7 was manufactured in the same manner as that of Example 1 except that a 25-nm-thick first emitting layer was formed as the emitting layer, the first electron transporting layer was formed on the first emitting layer without forming the second emitting layer, and the compound BH1-84 (first host material) in the first emitting layer was replaced with the first compound listed in Table 3.

### Comparative Example 4

As shown in Table 3, the organic EL device of Comparative Example 4 was manufactured in the same manner as that of Comparative Example 2 except that a compound BH1-87 (first host material) in the first emitting layer of Comparative Example 2 was replaced with the first compound listed in Table 3.

TABLE 3

| | First emitting layer | | | Second emitting layer | | | | |
|---|---|---|---|---|---|---|---|---|
| | | | Film | | | Film | | |
| | First | Third | thickness | Second | Fourth | thickness | EQE | LT95 |
| | compound | compound | [nm] | compound | compound | [nm] | [%] | [hr] |
| Ex. 5 | BH3-1 | BD2 | 5 | BH2-3 | BD2 | 20 | 9.8 | 290 |
| Ex. 6 | BH3-1 | BD2 | 5 | BH2 | BD2 | 20 | 9.9 | 230 |
| Ex. 7 | BH3-1 | BD2 | 5 | BH1-84 | BD2 | 20 | 9.0 | 250 |
| Ex. 8 | BH3-1 | BD2 | 5 | BH1-87 | BD2 | 20 | 9.2 | 240 |
| Ref. Ex. 5 | BH3-2 | BD2 | 5 | BH2 | BD2 | 20 | 10.0 | 140 |
| Ref. Ex. 6 | BH3-2 | BD2 | 5 | BH1-87 | BD2 | 20 | 9.5 | 140 |
| Ref. Ex. 7 | BH3-1 | BD2 | 25 | — | — | — | 8.4 | 110 |
| Ref. Ex. 2 | BH1-84 | BD2 | 25 | — | — | — | 7.2 | 80 |
| Comp. 1 | — | — | — | BH2-3 | BD2 | 25 | 8.5 | 145 |
| Comp. 4 | BH3-2 | BD2 | 25 | — | — | — | 8.5 | 60 |
| Comp. 2 | BH1-87 | BD2 | 25 | — | — | — | 7.3 | 75 |
| Comp. 3 | — | — | — | BH2 | BD2 | 25 | 8.8 | 78 |

### Examples 9 to 12

The organic EL devices of Examples 9 to 12 were each manufactured in the same manner as that of Example 1 except that at least one of the compound BH1-84 (first host material) in the first emitting layer or the compound BH2-3 in the second emitting layer was replaced with the compound listed in Table 4.

### Reference Examples 8 and 9

The organic EL devices of Reference Examples 8 and 9 were each manufactured in the same manner as that of Example 1 except that at least one of the compound BH1-84 (first host material) in the first emitting layer or the compound BH2-3 in the second emitting layer was replaced with the compound listed in Table 4.

### Reference Example 10

As shown in Table 4, the organic EL device of Reference Example 10 was manufactured in the same manner as that of Example 1 except that a 25-nm-thick first emitting layer was formed as the emitting layer, the first electron transporting layer was formed on the first emitting layer without forming the second emitting layer, and the compound BH1-84 (first host material) in the first emitting layer was replaced with the first compound listed in Table 4.

### Comparative Example 5

As shown in Table 4, the organic EL device of Comparative Example 5 was manufactured in the same manner as that of Comparative Example 2 except that the compound BH1-87 (first host material) in the first emitting layer of Comparative Example 2 was replaced with the first compound listed in Table 4.

TABLE 4

| | First emitting layer | | | Second emitting layer | | | | |
|---|---|---|---|---|---|---|---|---|
| | | | Film | | | Film | | |
| | First | Third | thickness | Second | Fourth | thickness | EQE | LT95 |
| | compound | compound | [nm] | compound | compound | [nm] | [%] | [hr] |
| Ex. 9 | BH4-1 | BD2 | 5 | BH2-3 | BD2 | 20 | 9.2 | 350 |
| Ex. 10 | BH4-1 | BD2 | 5 | BH2 | BD2 | 20 | 9.5 | 290 |
| Ex. 11 | BH4-1 | BD2 | 5 | BH3-1 | BD2 | 20 | 9.5 | 270 |
| Ex. 12 | BH4-1 | BD2 | 5 | BH3-2 | BD2 | 20 | 9.7 | 230 |
| Ref. Ex. 8 | BH4-2 | BD2 | 5 | BH2 | BD2 | 20 | 9.9 | 200 |
| Ref. Ex. 9 | BH4-2 | BD2 | 5 | BH3-2 | BD2 | 20 | 10.0 | 180 |
| Ref. Ex. 10 | BH4-1 | BD2 | 25 | — | — | — | 8.0 | 157 |
| Ref. Ex. 7 | BH3-1 | BD2 | 25 | — | — | — | 8.4 | 110 |
| Comp. 1 | — | — | — | BH2-3 | BD2 | 25 | 8.5 | 145 |
| Comp. 5 | BH4-2 | BD2 | 25 | — | — | — | 8.2 | 80 |
| Comp. 4 | BH3-2 | BD2 | 25 | — | — | — | 8.5 | 60 |
| Comp. 3 | — | — | — | BH2 | BD2 | 25 | 8.8 | 78 |

### Example 13

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. A film thickness of the ITO transparent electrode was 130 nm.

The cleaned glass substrate having the transparent electrode line was attached to a substrate holder of a vacuum deposition apparatus. Initially, a compound HA1 was vapor-deposited on a surface provided with the transparent electrode line to cover the transparent electrode, thereby forming a 10-nm-thick hole injecting layer (HI).

After the formation of the hole injecting layer, a compound HT2 was vapor-deposited to form an 80-nm-thick first hole transporting layer (HT).

After the formation of the first hole transporting layer, a compound HT3 was vapor-deposited to form a 5-nm-thick second hole transporting layer (also referred to as an electron blocking layer (EBL)).

A compound BH1-81 (first host material (BH)) and a compound BD1 (dopant material (BD)) were co-deposited on the second hole transporting layer such that the ratio of the compound BD1 accounted for 2 mass %, thereby forming a 5-nm-thick first emitting layer.

A compound BH2-11 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET7 was vapor-deposited on the second emitting layer to form a 3-nm-thick first electron transporting layer (also referred to as a hole blocking layer (HBL)).

A compound ET3 was vapor-deposited on the first electron transporting layer to form a 20-nm-thick second electron transporting layer (ET).

LiF was vapor-deposited on the second electron transporting layer to form a 1-nm-thick electron injecting layer.

Metal (Al) was vapor-deposited on the electron injecting layer to form an 80-nm-thick cathode.

A device arrangement of the organic EL device of Example 13 is roughly shown as follows.

### ITO(130)/HA1(10)/HT2(80)/HT3(5)/BH1-81:BD1(5.98%:2%)/BH2-11:BD1(20.98%:2%)/ET7(3)/ET3(20)/LiF(1)/Al(80)

Numerals in parentheses represent a film thickness (unit: nm).

The numerals (98%:2%) represented by percentage in the same parentheses indicate a ratio (mass %) between the host material (compound BH1-81 or BH2-11) and the compound BD1 in the first emitting layer or the second emitting layer. Similar notations apply to the description below.

### Examples 14 to 16

The organic EL devices of Examples 14 to 16 were each manufactured in the same manner as that of Example 13 except that compounds listed in Table 5 were used as the first compound (first host material) of the first emitting layer and the second compound (second host material) of the second emitting layer.

### Comparative Examples 6 to 8

As shown in Table 5, the organic EL devices of Comparative Examples 6 to 8 were each manufactured in the same manner as that of Example 13 except that a 25-nm-thick first emitting layer was formed as the emitting layer, the first electron transporting layer was formed on the first emitting layer without forming the second emitting layer, and the first compound listed in Table 5 was used as the first compound (first host material) of the first emitting layer.

TABLE 5

| | First emitting layer | | | Second emitting layer | | | | |
|---|---|---|---|---|---|---|---|---|
| | | | Film | | | Film | | |
| | First | Third | thickness | Second | Fourth | thickness | EQE | LT95 |
| | compound | compound | [nm] | compound | compound | [nm] | [%] | [hr] |
| Ex. 13 | BH1-81 | BD1 | 5 | BH2-11 | BD1 | 20 | 10.8 | 220 |
| Ex. 14 | BH1-82 | BD1 | 5 | BH2-12 | BD1 | 20 | 10.8 | 220 |
| Ex. 15 | BH1-88 | BD1 | 5 | BH2-11 | BD1 | 20 | 10.9 | 180 |
| Ex. 16 | BH1-88 | BD1 | 5 | BH2-12 | BD1 | 20 | 10.9 | 180 |
| Comp. 6 | BH1-81 | BD1 | 25 | — | — | — | 6.9 | 130 |
| Comp. 7 | BH1-82 | BD1 | 25 | — | — | — | 6.9 | 120 |
| Comp. 8 | BH1-88 | BD1 | 25 | — | — | — | 7.0 | 110 |

### Examples 17 to 22

The organic EL devices of Examples 17 to 22 were each manufactured in the same manner as that of Example 13 except that compounds listed in Table 6 were used as the first compound (first host material) of the first emitting layer and the second compound (second host material) of the second emitting layer.

### Comparative Examples 9 to 11

As shown in Table 6, the organic EL devices of Comparative Examples 9 to 11 were each manufactured in the same manner as that of Example 13 except that a 25-nm-thick first emitting layer was formed as the emitting layer, the first electron transporting layer was formed on the first emitting layer without forming the second emitting layer, and the compound listed in Table 6 was used as the first compound (first host material) of the first emitting layer.

TABLE 6

| | First emitting layer | | | Second emitting layer | | | | |
|---|---|---|---|---|---|---|---|---|
| | | | Film | | | Film | | |
| | First | Third | thickness | Second | Fourth | thickness | EQE | LT95 |
| | compound | compound | [nm] | compound | compound | [nm] | [%] | [hr] |
| Ex. 17 | BH1-84 | BD1 | 5 | BH2-13 | BD1 | 20 | 11.2 | 200 |
| Ex. 18 | BH1-84 | BD1 | 5 | BH2-16 | BD1 | 20 | 11.0 | 210 |
| Ex. 19 | BH1-86 | BD1 | 5 | BH2-14 | BD1 | 20 | 11.3 | 180 |
| Ex. 20 | BH1-87 | BD1 | 5 | BH2-13 | BD1 | 20 | 11.3 | 160 |
| Ex. 21 | BH1-87 | BD1 | 5 | BH2-14 | BD1 | 20 | 11.4 | 150 |
| Ex. 22 | BH1-87 | BD1 | 5 | BH2-16 | BD1 | 20 | 11.1 | 170 |
| Comp. 9 | BH1-84 | BD1 | 25 | — | — | — | 7.2 | 140 |
| Comp. 10 | BH1-86 | BD1 | 25 | — | — | — | 7.2 | 130 |
| Comp. 11 | BH1-87 | BD1 | 25 | — | — | — | 7.3 | 120 |

### Examples 23 to 26

The organic EL devices of Examples 23 to 26 were each manufactured in the same manner as that of Example 13 except that compounds listed in Table 7 were used as the first compound (first host material) of the first emitting layer and the second compound (second host material) of the second emitting layer.

### Comparative Examples 12 and 13

As shown in Table 7, the organic EL devices of Comparative Examples 12 and 13 were each manufactured in the same manner as that of Example 13 except that a 25-nm-thick first emitting layer was formed as the emitting layer, the first electron transporting layer was formed on the first emitting layer without forming the second emitting layer, and the compound listed in Table 7 was used as the first compound (first host material) of the first emitting layer.

TABLE 7

| | First emitting layer | | | Second emitting layer | | | | |
|---|---|---|---|---|---|---|---|---|
| | | | Film | | | Film | | |
| | First | Third | thickness | Second | Fourth | thickness | EQE | LT95 |
| | compound | compound | [nm] | compound | compound | [nm] | [%] | [hr] |
| Ex. 23 | BH1-83 | BD1 | 5 | BH2-15 | BD1 | 20 | 10.3 | 260 |
| Ex. 24 | BH1-83 | BD1 | 5 | BH2-17 | BD1 | 20 | 10.5 | 250 |
| Ex. 25 | BH1-89 | BD1 | 5 | BH2-15 | BD1 | 20 | 10.4 | 210 |
| Ex. 26 | BH1-89 | BD1 | 5 | BH2-17 | BD1 | 20 | 10.6 | 200 |
| Comp. 12 | BH1-83 | BD1 | 25 | — | — | — | 6.5 | 120 |
| Comp. 13 | BH1-89 | BD1 | 25 | — | — | — | 6.6 | 100 |

### Examples 27 and 28

The organic EL devices of Examples 27 and 28 were each manufactured in the same manner as that of Example 13 except that the film thickness of the first emitting layer was changed to 3 nm, the film thickness of the second emitting layer was changed to 15 nm, and compounds listed in Table 8 were used as the first compound (first host material) of the first emitting layer and the second compound (second host material) of the second emitting layer.

### Comparative Examples 14 and 15

As shown in Table 8, the organic EL devices of Comparative Examples 14 and 15 were each manufactured in the same manner as that of Example 13 except that an 18-nm-thick first emitting layer was formed as the emitting layer, the first electron transporting layer was formed on the first emitting layer without forming the second emitting layer, and the compound listed in Table 8 was used as the first compound (first host material) of the first emitting layer.

TABLE 8

| | First emitting layer | | | Second emitting layer | | | | |
|---|---|---|---|---|---|---|---|---|
| | | | Film | | | Film | | |
| | First | Third | thickness | Second | Fourth | thickness | EQE | LT95 |
| | compound | compound | [nm] | compound | compound | [nm] | [%] | [hr] |
| Ex. 27 | BH3-1 | BD1 | 3 | BH2-13 | BD1 | 15 | 9.7 | 180 |
| Ex. 28 | BH3-2 | BD1 | 3 | BH2-13 | BD1 | 15 | 9.8 | 130 |
| Comp. 14 | BH3-1 | BD1 | 18 | — | — | — | 8.8 | 80 |
| Comp. 15 | BH3-2 | BD1 | 18 | — | — | — | 8.9 | 60 |

### Examples 29 and 30

The organic EL devices of Examples 29 and 30 were each manufactured in the same manner as that of Example 13 except that the film thickness of the first emitting layer was changed to 8 nm, the film thickness of the second emitting layer was changed to 12 nm, and compounds listed in Table 9 were used as the first compound (first host material) of the first emitting layer and the second compound (second host material) of the second emitting layer.

### Comparative Examples 16 and 17

As shown in Table 9, the organic EL devices of Comparative Examples 16 and 17 were each manufactured in the same manner as that of Example 13 except that a 20-nm-thick first emitting layer was formed as the emitting layer, the first electron transporting layer was formed on the first emitting layer without forming the second emitting layer, and the compound listed in Table 9 was used as the first compound (first host material) of the first emitting layer.

TABLE 9

| | First emitting layer | | | Second emitting layer | | | | |
|---|---|---|---|---|---|---|---|---|
| | | | Film | | | Film | | |
| | First | Third | thickness | Second | Fourth | thickness | EQE | LT95 |
| | compound | compound | [nm] | compound | compound | [nm] | [%] | [hr] |
| Ex. 29 | BH4-1 | BD1 | 8 | BH2-16 | BD1 | 12 | 9.4 | 250 |
| Ex. 30 | BH4-2 | BD1 | 8 | BH2-16 | BD1 | 12 | 9.5 | 180 |
| Comp. 16 | BH4-1 | BD1 | 20 | — | — | — | 7.2 | 100 |
| Comp. 17 | BH4-2 | BD1 | 20 | — | — | — | 7.3 | 80 |

### Example 31

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. A film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 10-nm-thick hole injecting layer (HI).

After the formation of the hole injecting layer, the compound HT1 was vapor-deposited to form an 80-nm-thick first hole transporting layer (HT).

After the formation of the first hole transporting layer, a compound EBL was vapor-deposited to form a 10-nm-thick second hole transporting layer (also referred to as an electron blocking layer (EBL)).

A compound BH1-90 (first host material (BH)) and the compound BD2 (dopant material (BD)) were co-deposited on the second hole transporting layer such that the ratio of the compound BD2 accounted for 2 mass %, thereby forming a 12.5-nm-thick first emitting layer.

A compound BH2-18 (second host material (BH)) and the compound BD2 (dopant material (BD)) were co-deposited on the first emitting layer such that the ratio of the compound BD2 accounted for 2 mass %, thereby forming a 12.5-nm-thick second emitting layer.

A compound HBL was vapor-deposited on the second emitting layer to form a 3-nm-thick first electron transporting layer (also referred to as a hole blocking layer (HBL)).

The compound ET2 was vapor-deposited on the first electron transporting layer to form a 20-nm-thick second electron transporting layer (ET).

LiF was vapor-deposited on the second electron transporting layer to form a 1-nm-thick electron injecting layer.

Metal (Al) was vapor-deposited on the electron injecting layer to form an 80-nm-thick cathode.

A device arrangement of the organic EL device of Example 31 is roughly shown as follows.

### ITO(130)/HA1(10)/HT1(80)/EBL(10)/BH1-90:BD2(12.5, 98%:2%)/BH2-18:BD2(12.5, 98%:2%)/HBL(3)/ET2(20)/LiF(1)/Al(80)

Numerals in parentheses represent a film thickness (unit: nm).

The numerals (98%:2%) represented by percentage in the same parentheses indicate a ratio (mass %) between the host material (compound BH1-90 or BH2-18) and the compound BD2 in the first emitting layer or the second emitting layer. Similar notations apply to the description below.

### Examples 32 to 35

The organic EL devices of Examples 32 to 35 were each manufactured in the same manner as that of Example 31 except that the compound BH1-90 in the first emitting layer and the compound BH2-18 in the second emitting layer were replaced with the first compound and the second compound listed in Tables 11 to 14.

### Examples 36 to 38

The organic EL devices of Examples 36 to 38 were each manufactured in the same manner as that of Example 31 except that the compounds BH1-90 and BD2 in the first emitting layer and the compounds BH2-18 and BD2 in the second emitting layer were replaced with the first compound, the second compound, the third compound and the fourth compound listed in Tables 15 to 17.

### Reference Examples 11 to 15

The organic EL devices of Reference Examples 11 to 15 were each manufactured in the same manner as that of Example 31 except that the compound BH1-90 in the first emitting layer and the compound BH2-18 in the second emitting layer were replaced with the first compound and the second compound listed in Tables 10 to 14.

### Reference Examples 16 and 17

The organic EL devices of Reference Examples 16 and 17 were each manufactured in the same manner as that of Example 31 except that the compounds BH1-90 and BD2 in the first emitting layer and the compounds BH2-18 and BD2 in the second emitting layer were replaced with the first compound, the second compound, the third compound and the fourth compound listed in Tables 15 and 16.

### Comparative Examples 18 to 27

As shown in Tables 10 to 14, the organic EL devices of Comparative Examples 18 to 27 were each manufactured in the same manner as that of Example 31 except that a 25-nm-thick second emitting layer was formed as the emitting layer on the second hole transporting layer without forming the first emitting layer, and the compound listed in Tables 10 to 14 was used as the second compound (second host material) of the second emitting layer.

### Comparative Examples 28 to 32

As shown in Tables 15 to 17, the organic EL devices of Comparative Examples 28 to 32 were manufactured in the same manner as that of Example 31 except that a 25-nm-thick second emitting layer was formed as the emitting layer on the second hole transporting layer without forming the first emitting layer, and the compound listed in Table 10 was used as the second compound (second host material) and the fourth compound.

In order to easily compare Examples, Reference Examples, and Comparative Examples, some of Reference Examples and Comparative Examples are repeatedly listed in Tables 10 to 17.

TABLE 10

| | First emitting layer | | | Second emitting layer | | | | |
|---|---|---|---|---|---|---|---|---|
| | | | Film | | | Film | | |
| | First | Third | thickness | Second | Fourth | thickness | EQE | LT95 |
| | compound | compound | [nm] | compound | compound | [nm] | [%] | [hr] |
| Ex. 31 | BH1-90 | BD2 | 12.5 | BH2-18 | BD2 | 12.5 | 10.4 | 164 |
| Ref. Ex. 11 | BH1-92 | BD2 | 12.5 | BH2-28 | BD2 | 12.5 | 10.5 | 110 |
| Comp. 18 | — | — | — | BH2-18 | BD2 | 25 | 9.5 | 109 |
| Comp. 19 | — | — | — | BH2-28 | BD2 | 25 | 9.7 | 93 |

TABLE 11

| | First emitting layer | | | Second emitting layer | | | | |
|---|---|---|---|---|---|---|---|---|
| | | | Film | | | Film | | |
| | First | Third | thickness | Second | Fourth | thickness | EQE | LT95 |
| | compound | compound | [nm] | compound | compound | [nm] | [%] | [hr] |
| Ex. 32 | BH1-91 | BD2 | 12.5 | BH2-19 | BD2 | 12.5 | 10.0 | 187 |
| Ref. Ex. 12 | BH1-93 | BD2 | 12.5 | BH2-29 | BD2 | 12.5 | 9.8 | 125 |
| Comp. 20 | — | — | — | BH2-19 | BD2 | 25 | 9.1 | 121 |
| Comp. 11 | — | — | — | BH2-29 | BD2 | 25 | 9.2 | 103 |

TABLE 12

| | First emitting layer | | | Second emitting layer | | | | |
|---|---|---|---|---|---|---|---|---|
| | | | Film | | | Film | | |
| | First | Third | thickness | Second | Fourth | thickness | EQE | LT95 |
| | compound | compound | [nm] | compound | compound | [nm] | [%] | [hr] |
| Ex. 33 | BH1-92 | BD2 | 12.5 | BH2-20 | BD2 | 12.5 | 10.0 | 160 |
| Ref. Ex. 13 | BH1-92 | BD2 | 12.5 | BH2-30 | BD2 | 12.5 | 10.3 | 124 |
| Comp. 22 | — | — | — | BH2-20 | BD2 | 25 | 9.1 | 120 |
| Comp. 23 | — | — | — | BH2-30 | BD2 | 25 | 9.4 | 101 |

TABLE 13

| | First emitting layer | | | Second emitting layer | | | | |
|---|---|---|---|---|---|---|---|---|
| | | | Film | | | Film | | |
| | First | Third | thickness | Second | Fourth | thickness | EQE | LT95 |
| | compound | compound | [nm] | compound | compound | [nm] | [%] | [hr] |
| Ex. 34 | BH1-93 | BD2 | 12.5 | BH2-21 | BD2 | 12.5 | 10.1 | 159 |
| Ref. Ex. 14 | BH1-93 | BD2 | 12.5 | BH2-31 | BD2 | 12.5 | 10.3 | 124 |
| Comp. 24 | — | — | — | BH2-21 | BD2 | 25 | 9.0 | 123 |
| Comp. 25 | — | — | — | BH2-31 | BD2 | 25 | 9.2 | 103 |

TABLE 14

| | First emitting layer | | | Second emitting layer | | | | |
|---|---|---|---|---|---|---|---|---|
| | | | Film | | | Film | | |
| | First | Third | thickness | Second | Fourth | thickness | EQE | LT95 |
| | compound | compound | [nm] | compound | compound | [nm] | [%] | [hr] |
| Ex. 35 | BH1-94 | BD2 | 12.5 | BH2-22 | BD2 | 12.5 | 9.4 | 158 |
| Ref. Ex. 15 | BH1-94 | BD2 | 12.5 | BH2-32 | BD2 | 12.5 | 9.7 | 124 |
| Comp. 26 | — | — | — | BH2-22 | BD2 | 25 | 8.9 | 122 |
| Comp. 27 | — | — | — | BH2-32 | BD2 | 25 | 9.2 | 103 |

TABLE 15

| | First emitting layer | | | Second emitting layer | | | | |
|---|---|---|---|---|---|---|---|---|
| | | | Film | | | Film | | |
| | First | Third | thickness | Second | Fourth | thickness | EQE | LT95 |
| | compound | compound | [nm] | compound | compound | [nm] | [%] | [hr] |
| Ex. 36 | BH1-90 | BD3 | 12.5 | BH2-14 | BD3 | 12.5 | 9.8 | 177 |
| Ref. Ex. 16 | BH1-92 | BD3 | 12.5 | BH2-33 | BD3 | 12.5 | 9.9 | 125 |
| Comp. 28 | — | — | — | BH2-14 | BD3 | 25 | 8.9 | 118 |
| Comp. 29 | — | — | — | BH2-33 | BD3 | 25 | 9.0 | 102 |

TABLE 16

| | First emitting layer | | | Second emitting layer | | | | |
|---|---|---|---|---|---|---|---|---|
| | | | Film | | | Film | | |
| | First | Third | thickness | Second | Fourth | thickness | EQE | LT95 |
| | compound | compound | [nm] | compound | compound | [nm] | [%] | [hr] |
| Ex. 37 | BH1-91 | BD3 | 12.5 | BH2-23 | BD3 | 12.5 | 10.9 | 193 |
| Ref. Ex. 17 | BH1-93 | BD3 | 12.5 | BH2-34 | BD3 | 12.5 | 10.9 | 127 |
| Comp. 30 | — | — | — | BH2-23 | BD3 | 25 | 9.9 | 124 |
| Comp. 31 | — | — | — | BH2-34 | BD3 | 25 | 10.1 | 108 |

TABLE 17

| | First emitting layer | | | Second emitting layer | | | | |
|---|---|---|---|---|---|---|---|---|
| | | | Film | | | Film | | |
| | First | Third | thickness | Second | Fourth | thickness | EQE | LT95 |
| | compound | compound | [nm] | compound | compound | [nm] | [%] | [hr] |
| Ex. 38 | BH1-92 | BD3 | 12.5 | BH2-24 | BD3 | 12.5 | 9.8 | 161 |
| Ref. Ex. 16 | BH1-92 | BD3 | 12.5 | BH2-33 | BD3 | 12.5 | 9.9 | 125 |
| Comp. 32 | — | — | — | BH2-24 | BD3 | 25 | 8.9 | 121 |
| Comp. 29 | — | — | — | BH2-33 | BD3 | 25 | 9.0 | 102 |

As shown in Tables 10 to 17, when the compound of the second emitting layer used as the second host material is a compound having at least one deuterium atom, the organic EL device has a long lifetime. An increase in lifetime of the organic EL devices in which the first and second emitting layers were laminated as shown in Examples 31 to 38 was greater than that of the organic EL device only including the second emitting layer as the emitting layer.

### Evaluation of Compounds

Preparation of Toluene Solution

The compound BD1 was dissolved in toluene at a concentration of 4.9×10⁻⁶ mol/L to prepare a toluene solution of the compound BD1.

The compound BD2 was dissolved in toluene at a concentration of 4.9×10⁻⁶ mol/L to prepare a toluene solution of the compound BD2.

The compound BD3 was dissolved in toluene at a concentration of 4.9×10⁻⁶ mol/L to prepare a toluene solution of the compound BD3.

### Measurement of Maximum Fluorescence Peak Wavelength (FL-peak)

Using a fluorescence spectrometer (spectrophotofluorometer F-7000 manufactured by Hitachi High-Tech Science Corporation), the toluene solution of the compound BD1, the toluene solution of the compound BD2, and the toluene solution of the compound BD3 were respectively excited at 390 nm, where a maximum fluorescence peak wavelength was measured.

The maximum fluorescence peak wavelength of the compound BD1 was 451 nm.

The maximum fluorescence peak wavelength of the compound BD2 was 455 nm.

The maximum fluorescence peak wavelength of the compound BD3 was 458 nm.

### Triplet Energy T₁

A measurement target compound was dissolved in EPA (diethylether:isopentane:ethanol=5:5:2 in volume ratio) at a concentration of 10 μmol/L, and the obtained solution was put in a quartz cell to provide a measurement sample. A phosphorescence spectrum (ordinate axis: phosphorescent luminous intensity, abscissa axis: wavelength) of the measurement sample is measured at a low temperature (77K). A tangent is drawn to the rise of the phosphorescence spectrum close to the short-wavelength region. An energy amount is calculated by a conversion equation (F1) below on a basis of a wavelength value λ_{edge} [nm] at an intersection of the tangent and the abscissa axis. The calculated energy amount is defined as triplet energy T₁.

T₁ [eV]=1239.85/λ_{edge} Conversion Equation (F1):

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 manufactured by Hitachi High-Technologies Corporation was used.

Table 18 shows measurement results of triplet energy T₁ of the respective compounds.

TABLE 18

| Compound | T₁ [eV] |
|---|---|
| BH1-81 | 2.08 |
| BH1-82 | 2.08 |
| BH1-83 | 2.09 |
| BH1-84 | 2.09 |
| BH1-85 | 2.09 |
| BH1-86 | 2.09 |
| BH1-87 | 2.09 |
| BH1-88 | 2.08 |
| BH1-89 | 2.09 |
| BH1-90 | 2.09 |
| BH1-91 | 2.09 |
| BH1-92 | 2.09 |
| BH1-93 | 2.09 |
| BH1-94 | 2.13 |
| BH3-1 | 2.10 |
| BH3-2 | 2.10 |
| BH4-1 | 2.25 |
| BH4-2 | 2.25 |
| BH2-3 | 1.87 |
| BH2 | 1.87 |
| BH2-11 | 1.81 |
| BH2-12 | 1.81 |
| BH2-13 | 1.81 |
| BH2-14 | 1.81 |
| BH2-15 | 1.81 |
| BH2-16 | 1.81 |
| BH2-17 | 1.81 |
| BH2-18 | 1.83 |
| BH2-19 | 1.81 |
| BH2-20 | 1.81 |
| BH2-21 | 1.81 |
| BH2-22 | 1.81 |
| BH2-23 | 1.81 |
| BH2-24 | 1.81 |
| BH2-28 | 1.83 |
| BH2-29 | 1.81 |
| BH2-30 | 1.81 |
| BH2-31 | 1.81 |
| BH2-32 | 1.81 |
| BH2-33 | 1.81 |
| BH2-34 | 1.81 |
| BD1 | 2.61 |
| BD2 | 2.64 |
| BD3 | 2.45 |

### Synthesis of Compound

### Synthesis Example 1: Synthesis of Compound BH2-11

The compound BH2-11 was synthesized in accordance with a synthesis scheme below.

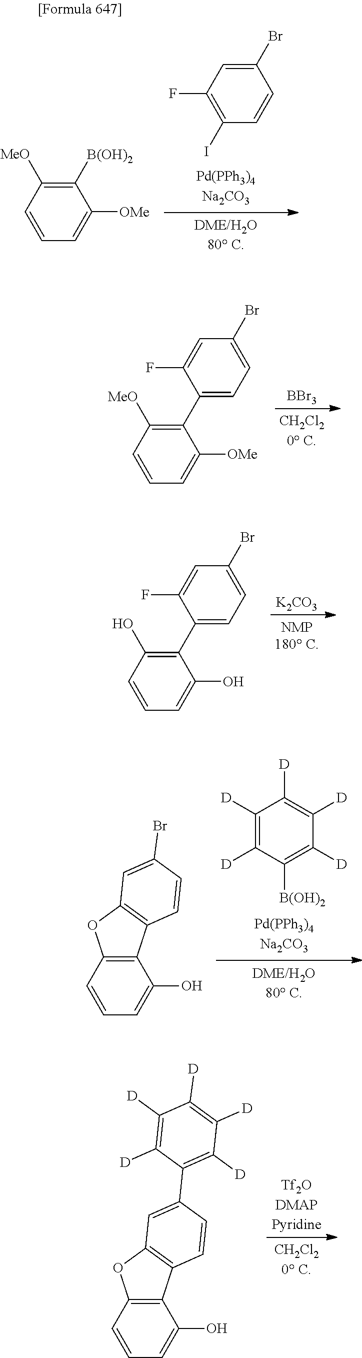

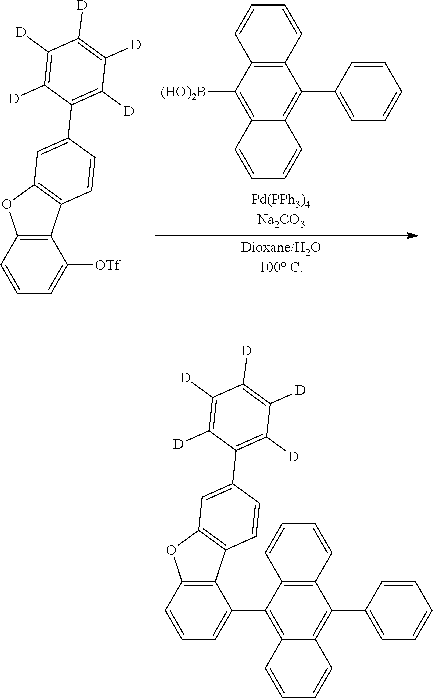 [SMILES: [2H]c1c([2H])c([2H])c(-c2ccc3c(c2)oc2cccc(OS(=O)(=O)C(F)(F)F)c23)c([2H])c1[2H] ; OB(O)c1c2ccccc2c(-c2ccccc2)c2ccccc12 ; [2H]c1c([2H])c([2H])c(-c2ccc3c(c2)oc2cccc(-c4c5ccccc5c(-c5ccccc5)c5ccccc45)c23)c([2H])c1[2H]]

### (1) Synthesis of 4-bromo-2-fluoro-2′,6′-dimethoxy-1,1′-biphenyl

Under argon atmosphere, 20.0 g of (2,6-dimethoxyphenyl)boronic acid, 39.7 g of 4-bromo-2-fluoro-1-iodobenzene, 2.54 g of tetrakis (triphenylphosphine)palladium(0), 385 mL of 1,2-dimethoxyethane, and 165 ml of 2M sodium carbonate aqueous solution were put into a flask and stirred at reflux for eight hours. The reaction solution was cooled to room temperature, and then extracted with toluene. After an aqueous phase was removed, an organic phase was washed with saturated saline. The organic phase was dried with anhydrous sodium sulfate and then concentrated. The residue was purified by silica gel column chromatography, and the obtained sample was dried in a vacuum at room temperature for three hours to obtain 25.2 g of 4-bromo-2-fluoro-2′,6′-dimethoxy-1,1′-biphenyl (a yield of 74%).

### (2) Synthesis of 4′-bromo-2′-fluoro-[1,1′-biphenyl]-2,6-diol

Under argon atmosphere, 25.2 g of 4-bromo-2-fluoro-2′,6′-dimethoxy-1,1′-biphenyl and 162 mL of (dehydrated) dichloromethane were put into a flask, and cooled to 0 degrees C. Boron tribromide dichloromethane solution (1.0 mol/I, 243 mL) was added thereto, and the reaction solution was stirred at room temperature for four hours. After the reaction, the solution was cooled to −78 degrees C. The solution was carefully deactivated with methanol and then deactivated with a sufficient amount of water. The reaction solution was extracted with dichloromethane. After an aqueous phase was removed, an organic phase was washed with saturated saline. The organic phase was dried with anhydrous sodium sulfate, and then origin impurities were removed through a silica gel short column. The solution was concentrated and the obtained sample was dried in a vacuum at room temperature for three hours to obtain 21.6 g of 4′-bromo-2′-fluoro-[1,1′-biphenyl]-2,6-diol (a yield of 94%).

### (3) Synthesis of 7-bromodibenzo[b,d]furan-1-ol

Under argon atmosphere, 21.6 g of 4′-bromo-2′-fluoro-[1,1′-biphenyl]-2,6-diol, 450 mL of (dehydrated)N-methyl-2-pyrrolidinone, and 21.1 g of K₂CO₃ were put into a flask, and stirred at 180 degrees C. for 2 hours. After the reaction, the solution was cooled to room temperature. The reaction solution was extracted with ethyl acetate. After an aqueous phase was removed, an organic phase was washed with saturated saline. The organic phase was dried with anhydrous sodium sulfate and then purified by silica gel column chromatography. The obtained sample was dried in a vacuum at room temperature for three hours to obtain 13.4 g of 7-bromodibenzo[b,d]furan-1-ol (a yield of 67%).

### (4) Synthesis of 7-(phenyl-d5)dibenzo[b,d]furan-1-ol

Under argon atmosphere, 13.4 g of 7-bromodibenzo[b,d]furan-1-ol, 7.06 g of (phenyl-d5)boronic acid, 1.17 g of tetrakis (triphenylphosphine)palladium(0), 177 mL of 1,2-dimethoxyethane, and 76 ml of 2M sodium carbonate aqueous solution were put into a flask and stirred at reflux for eight hours. The reaction solution was cooled to room temperature, and then extracted with toluene. After an aqueous phase was removed, an organic phase was washed with saturated saline. The organic phase was dried with anhydrous sodium sulfate and then concentrated. The residue was purified by silica gel column chromatography. The obtained sample was dried in a vacuum at room temperature for three hours to obtain 11.7 g of 7-(phenyl-d5)dibenzo[b,d]furan-1-ol (a yield of 87%).

### (5) Synthesis of 7-(phenyl-d5)dibenzo[b,d]furan-1-yl trifluoromethanesulfonate

Under argon atmosphere, 11.7 g of 7-(phenyl-d5)dibenzo[b,d]furan-1-ol, 540 mg of N,N-dimethyl-4-aminopyridine, 14.9 g of trifluoro methane sulfonic acid anhydride, and 27 mL of (dehydrated) dichloromethane were put into a flask, and cooled to 0 degrees C. 5.34 mL of (dehydrated) pyridine was added dropwise and then stirred at room temperature for two hours. After the reaction, the solution was deactivated with a sufficient amount of water. The reaction solution was extracted with dichloromethane. After an aqueous phase was removed, an organic phase was washed with saturated saline. The organic phase was dried with anhydrous sodium sulfate, and then origin impurities were removed through a silica gel short column. The solution was concentrated and the obtained sample was dried in a vacuum at room temperature for three hours to obtain 16.1 g of 7-(phenyl-d5)dibenzo[b,d]furan-1-yl trifluoromethanesulfonate as a white solid (a yield of 92%).

### (6) Synthesis of 7-(phenyl-d5)-1-(10-phenylanthracen-9-yl)dibenzo[b,d]furan

Under argon atmosphere, 16.1 g of 7-(phenyl-d5)dibenzo[b,d]furan-1-yl trifluoromethanesulfonate, 12.7 g of (10-phenylanthracen-9-yl)boronic acid, 936 mg of tetrakis(triphenylphosphine)palladium(0), 142 mL of 1,4-dioxane, and 61 ml of 2M sodium carbonate aqueous solution were put into a flask and stirred at reflux for eight hours. The reaction solution was cooled to room temperature, and then extracted with toluene. After an aqueous phase was removed, an organic phase was washed with saturated saline. The organic phase was dried with anhydrous sodium sulfate and then concentrated. The residue was purified by silica gel column chromatography. The obtained sample was dried in a vacuum at 60 degrees C. for three hours to obtain 11.4 g of 7-(phenyl-d5)-1-(10-phenylanthracen-9-yl)dibenzo[b,d]furan (a yield of 56%). As a result of mass spectroscopy analysis, the solid was the compound 2-11, and m/e was equal to 501 while a calculated molecular weight was 501.64.

### Synthesis Example 2: Synthesis of Compound BH2-12

A compound BH2-12 was synthesized in accordance with a synthesis scheme below.

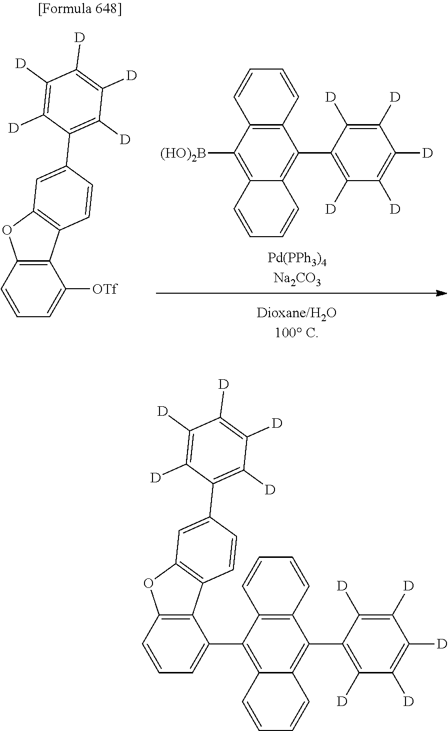 [SMILES: [2H]c1c([2H])c([2H])c(-c2ccc3c(c2)oc2cccc(OS(=O)(=O)C(F)(F)F)c23)c([2H])c1[2H] ; [2H]c1c([2H])c([2H])c(-c2c3ccccc3c(B(O)O)c3ccccc23)c([2H])c1[2H] ; [2H]c1c([2H])c([2H])c(-c2ccc3c(c2)oc2cccc(-c4c5ccccc5c(-c5c([2H])c([2H])c([2H])c([2H])c5[2H])c5ccccc45)c23)c([2H])c1[2H]]

In Synthesis Example 2, the compound BH2-12 was synthesized using (10-(phenyl-d5)anthracen-9-yl)boronic acid synthesized by a known method in place of (10-phenylanthracen-9-yl)boronic acid. Except for the above, the same reaction as in (6) of Synthesis Example 1 was performed to obtain a white solid. As a result of mass spectroscopy analysis, the white solid was the compound BH2-12, and m/e was equal to 506 while a calculated molecular weight was 506.67.

### Synthesis Example 3: Synthesis of Compound BH2-13

A compound BH2-13 was synthesized in accordance with a synthesis scheme below.

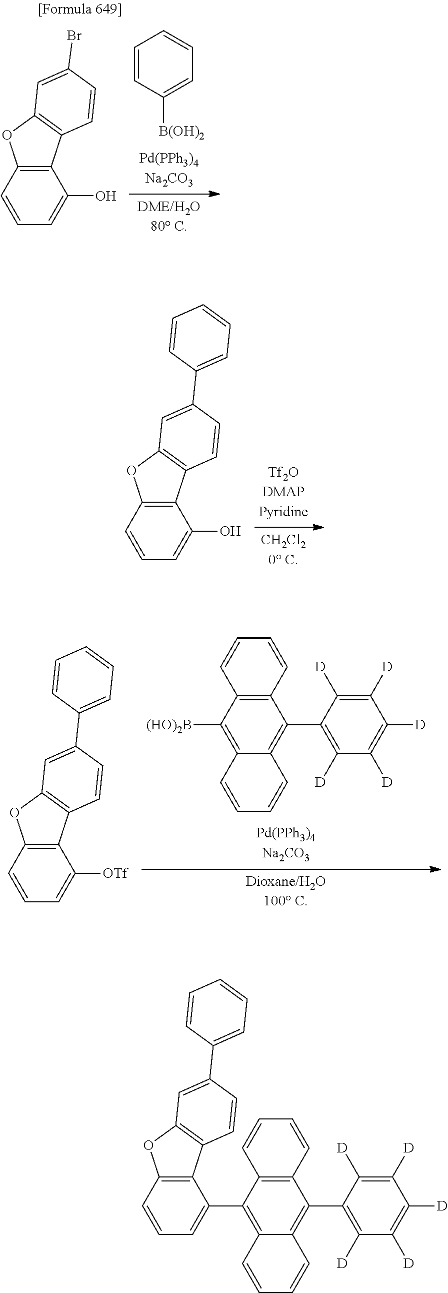

In Synthesis Example 3, the compound BH2-13 was synthesized using (phenyl)boronic acid in place of (phenyl-d5)boronic acid. Except for the above, the same reaction as in Synthesis Example 1 or 2 was performed to obtain a white solid. As a result of mass spectroscopy analysis, the white solid was the compound BH2-13, and m/e was equal to 501 while a calculated molecular weight was 501.64.

### Synthesis Example 4: Synthesis of Compound BH2-14

A compound BH2-14 was synthesized in accordance with a synthesis scheme below.

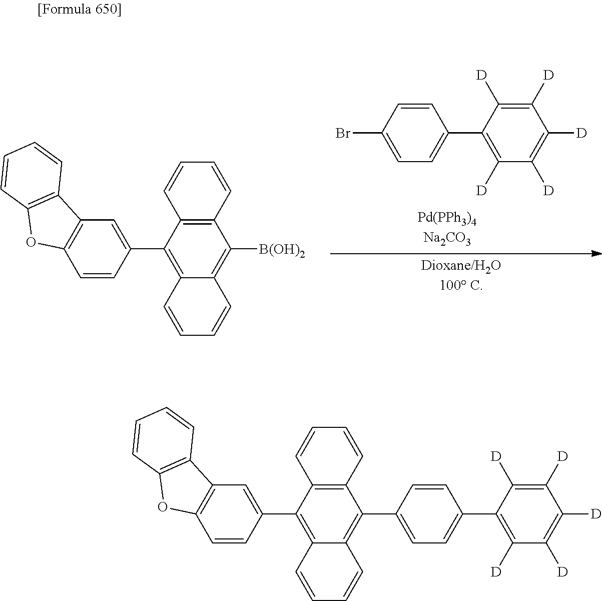 [SMILES: OB(O)c1c2ccccc2c(-c2ccc3oc4ccccc4c3c2)c2ccccc12 ; [2H]c1c([2H])c([2H])c(-c2ccc(Br)cc2)c([2H])c1[2H] ; [2H]c1c([2H])c([2H])c(-c2ccc(-c3c4ccccc4c(-c4ccc5oc6ccccc6c5c4)c4ccccc34)cc2)c([2H])c1[2H]]

### (1) Synthesis of 7-(phenyl-d5)-1-(10-phenylanthracen-9-yl)dibenzo[b,d]furan

Under argon atmosphere, 4.32 g of 4-bromo-1,1′-biphenyl-2′,3′,4′,5′,6′-d5, 7.39 g of (10-(dibenzo[b,d]furan-2-yl)anthracen-9-yl)boronic acid, 419 mg of tetrakis(triphenylphosphine)palladium(0), 64 mL of 1,4-dioxane, and 27 m1 of 2M sodium carbonate aqueous solution were put into a flask and stirred at reflux for eight hours. The reaction solution was cooled to room temperature, and then extracted with toluene. After an aqueous phase was removed, an organic phase was washed with saturated saline. The organic phase was dried with anhydrous sodium sulfate and then concentrated. The residue was purified by silica gel column chromatography to obtain 5.58 g of 2-(10-([1,1′-biphenyl]-4-yl-2′,3′,4′,5′,6′-d5)anthracen-9-yl)dibenzo[b,d]furan (a yield of 61%). As a result of mass spectroscopy analysis, the solid was the compound BH2-14, and m/e was equal to 501 while a calculated molecular weight was 501.64.

### Synthesis Example 5: Synthesis of Compound BH2-15

A compound BH2-15 was synthesized in accordance with a synthesis scheme below.

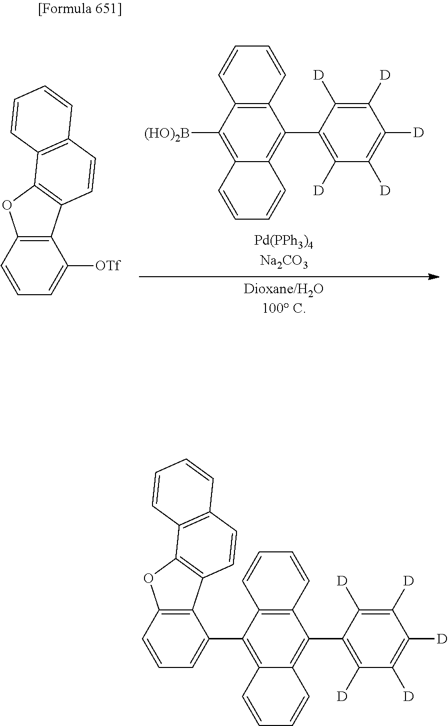 [SMILES: O=S(=O)(Oc1cccc2oc3c4ccccc4ccc3c12)C(F)(F)F ; [2H]c1c([2H])c([2H])c(-c2c3ccccc3c(B(O)O)c3ccccc23)c([2H])c1[2H] ; [2H]c1c([2H])c([2H])c(-c2c3ccccc3c(-c3cccc4oc5c6ccccc6ccc5c34)c3ccccc23)c([2H])c1[2H]]

In Synthesis Example 5, the compound BH2-15 was synthesized using naphtho[1,2-b]benzofuran-7-yl trifluoromethanesulfonate synthesized by a known method in place of 7-(phenyl-d5)dibenzo[b,d]furan-1-yl trifluoromethanesulfonate. Except for the above, the same reaction as in (6) of Synthesis Example 1 was performed to obtain a white solid. As a result of mass spectroscopy analysis, the white solid was the compound BH2-15, and m/e was equal to 475 while a calculated molecular weight was 475.60.

### Synthesis Example 6: Synthesis of Compound BH2-16

A compound BH2-16 was synthesized in accordance with a synthesis scheme below.

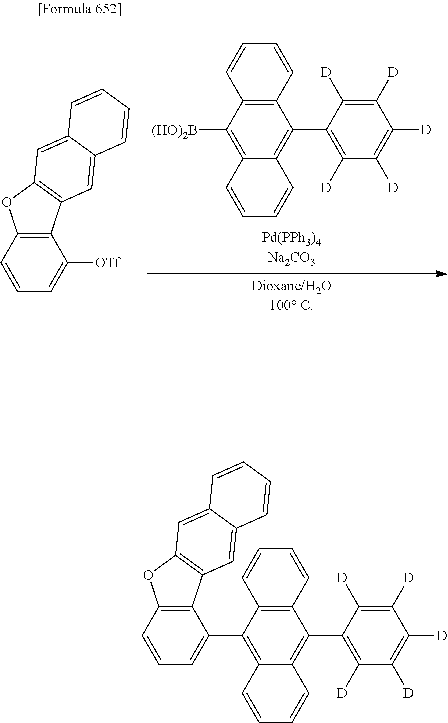 [SMILES: O=S(=O)(Oc1cccc2oc3cc4ccccc4cc3c12)C(F)(F)F ; [2H]c1c([2H])c([2H])c(-c2c3ccccc3c(B(O)O)c3ccccc23)c([2H])c1[2H] ; [2H]c1c([2H])c([2H])c(-c2c3ccccc3c(-c3cccc4oc5cc6ccccc6cc5c34)c3ccccc23)c([2H])c1[2H]]

In Synthesis Example 6, the compound BH2-16 was synthesized using naphtho[2,3-b]benzofuran-1-yl trifluoromethanesulfonate synthesized by a known method in place of naphtho[1,2-b]benzofuran-7-yl trifluoromethanesulfonate. Except for the above, the same reaction as in Synthesis Example 2 was performed to obtain a white solid. As a result of mass spectroscopy analysis, the white solid was the compound BH2-16, and m/e was equal to 475 while a calculated molecular weight was 475.60.

### Synthesis Example 7: Synthesis of Compound BH2-17

A compound BH2-17 was synthesized in accordance with a synthesis scheme below.

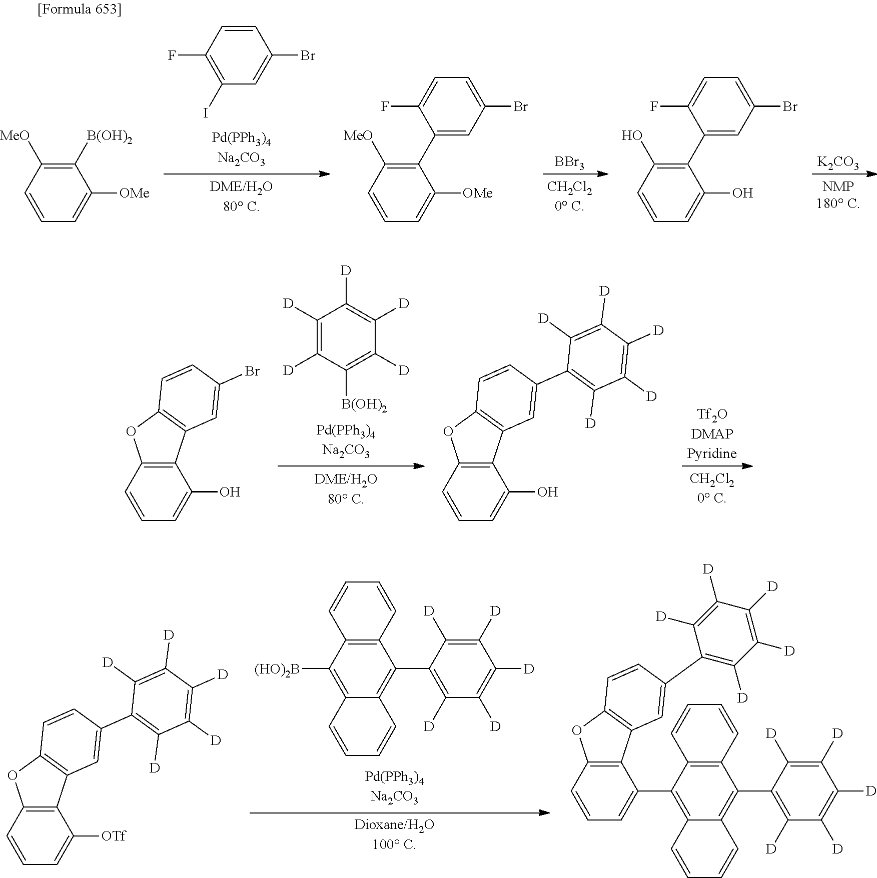

In Synthesis Example 7, the compound BH2-17 was synthesized using 4-bromo-1-fluoro-2-iodobenzene in place of 4-bromo-2-fluoro-1-iodobenzene. Except for the above, the same reaction as in Synthesis Example 1 or 2 was performed to obtain a white solid. As a result of mass spectroscopy analysis, the white solid was the compound BH2-17, and m/e was equal to 506 while a calculated molecular weight was 506.67.

### Synthesis Example 8: Synthesis of Compound BH1-81

The compound BH1-81 was synthesized in accordance with a synthesis scheme below.

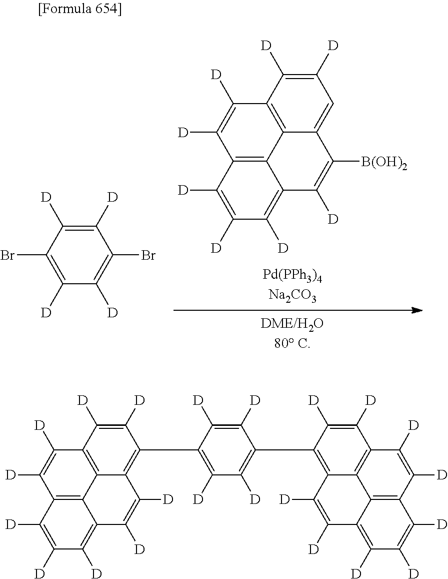 [SMILES: [2H]c1c([2H])c(Br)c([2H])c([2H])c1Br ; [2H]c1cc2c(B(O)O)c([2H])c3c([2H])c([2H])c([2H])c4c([2H])c([2H])c(c1[2H])c2c34 ; [2H]c1c([2H])c(-c2c([2H])c([2H])c3c([2H])c([2H])c4c([2H])c([2H])c([2H])c5c([2H])c([2H])c2c3c45)c([2H])c([2H])c1-c1c([2H])c([2H])c2c([2H])c([2H])c3c([2H])c([2H])c([2H])c4c([2H])c([2H])c1c2c34]

### (1) Synthesis of 1,1′-(1,4-phenylene-d4)bis(pyrene-2,3,4,5,6,7,8,9,10-d9)

Under argon atmosphere, 3.00 g of 1,4-dibromobenzene-2,3,5,6-d4, 6.22 g of (pyren-1-yl-d9)boronic acid, 578 mg of tetrakis(triphenylphosphine)palladium(0), 44 mL of 1,2-dimethoxyethane, and 19 m1 of 2M sodium carbonate aqueous solution were put into a flask and stirred at reflux for eight hours. The reaction solution was cooled to room temperature, and then extracted with toluene. After an aqueous phase was removed, an organic phase was washed with saturated saline. The organic phase was dried with anhydrous sodium sulfate and then concentrated. The residue was purified by silica gel column chromatography. The obtained sample was dried in a vacuum at room temperature for three hours to obtain 3.32 g of 1,1′-(1,4-phenylene-d4)bis(pyrene-2,3,4,5,6,7,8,9,10-d9) (a yield of 53%). As a result of mass spectroscopy analysis, the solid was the compound BH1-81, and m/e was equal to 500 while a calculated molecular weight was 500.73.

### Synthesis Example 9: Synthesis of Compound BH1-82

A compound BH1-82 was synthesized in accordance with a synthesis scheme below.

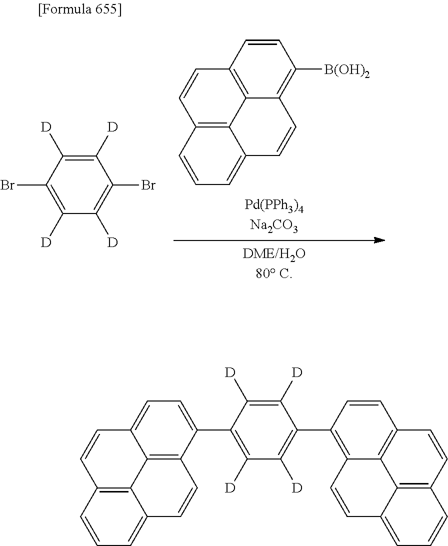

In Synthesis Example 9, the compound BH1-82 was synthesized using pyren-1-ylboronic acid synthesized by a known method in place of (pyren-1-yl-d9)boronic acid. Except for the above, the same reaction as in Synthesis Example 8 was performed to obtain a white solid. As a result of mass spectroscopy analysis, the white solid was the compound BH1-82, and m/e was equal to 482 while a calculated molecular weight was 482.62.

### Synthesis Example 10: Synthesis of Compound BH1-84

The compound BH1-84 was synthesized in accordance with a synthesis scheme below.

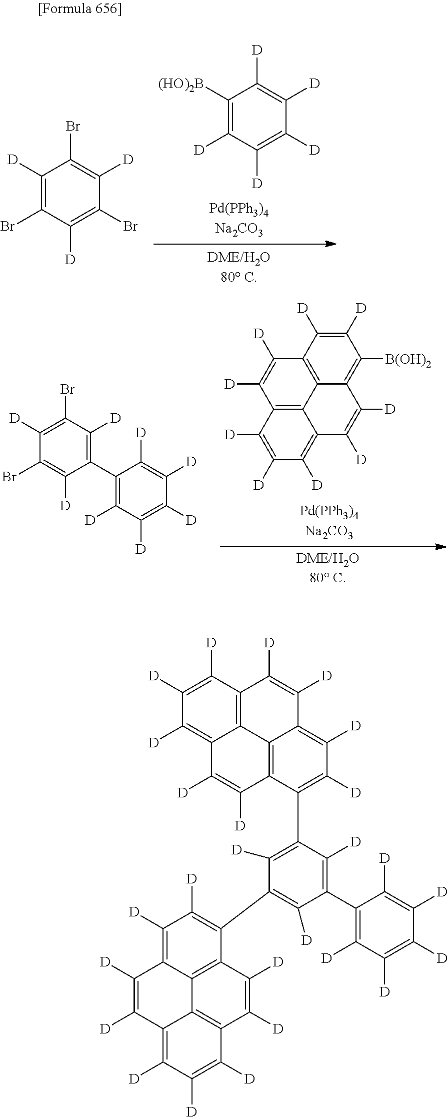 [SMILES: [2H]c1c(Br)c([2H])c(Br)c([2H])c1Br ; [2H]c1c([2H])c([2H])c(B(O)O)c([2H])c1[2H] ; [2H]c1c([2H])c([2H])c(-c2c([2H])c(Br)c([2H])c(Br)c2[2H])c([2H])c1[2H] ; [2H]c1c([2H])c2c([2H])c([2H])c3c([2H])c([2H])c(B(O)O)c4c([2H])c([2H])c(c1[2H])c2c34 ; [2H]c1c([2H])c([2H])c(-c2c([2H])c(-c3c([2H])c([2H])c4c([2H])c([2H])c5c([2H])c([2H])c([2H])c6c([2H])c([2H])c3c4c56)c([2H])c(-c3c([2H])c([2H])c4c([2H])c([2H])c5c([2H])c([2H])c([2H])c6c([2H])c([2H])c3c4c56)c2[2H])c([2H])c1[2H]]

### (1) Synthesis of 3,5-dibromo-1,1′-biphenyl-2,2′,3′,4,4′,5′,6,6′-d8

Under argon atmosphere, 3.17 g of 1,3,5-tribromobenzene-2,4,6-d3, 1.3 g of (phenyl-d5)boronic acid, 461 mg of tetrakis(triphenylphosphine)palladium(0), 35 mL of 1,2-dimethoxyethane, and 15 ml of 2M sodium carbonate aqueous solution were put into a flask and stirred at reflux for eight hours. The reaction solution was cooled to room temperature, and then extracted with toluene. After an aqueous phase was removed, an organic phase was washed with saturated saline. The organic phase was dried with anhydrous sodium sulfate and then concentrated. The residue was purified by silica gel column chromatography. The obtained sample was dried in a vacuum at room temperature for three hours to obtain 1.34 g of 1,1′-(1,4-phenylene-d4)bis(pyrene-2,3,4,5,6,7,8,9,10-d9) (a yield of 42%).

### (2) Synthesis of 1,1′-([1,1′-biphenyl]-3,5-diyl-d8)bis(pyrene-2,3,4,5,6,7,8,9,10-d9)

Under argon atmosphere, 13.4 g of 1,1′-(1,4-phenylene-d4)bis(pyrene-2,3,4,5,6,7,8,9,10-d9), 2.08 g of (pyren-1-yl-d9)boronic acid, 194 mg of tetrakis(triphenylphosphine)palladium(0), 15 mL of 1,2-dimethoxyethane, and 6.3 ml of 2M sodium carbonate aqueous solution were put into a flask and stirred at reflux for eight hours. The reaction solution was cooled to room temperature, and then extracted with toluene. After an aqueous phase was removed, an organic phase was washed with saturated saline. The organic phase was dried with anhydrous sodium sulfate and then concentrated. The residue was purified by silica gel column chromatography. The obtained sample was dried in a vacuum at room temperature for three hours to obtain 1.17 g of 1,1′-([1,1′-biphenyl]-3,5-diyl-d8)bis(pyrene-2,3,4,5,6,7,8,9,10-d9) (a yield of 48%). As a result of mass spectroscopy analysis, the solid was the compound BH1-84, and m/e was equal to 581 while a calculated molecular weight was 580.85.

### Synthesis Example 11: Synthesis of Compound BH1-86

A compound BH1-86 was synthesized in accordance with a synthesis scheme below.

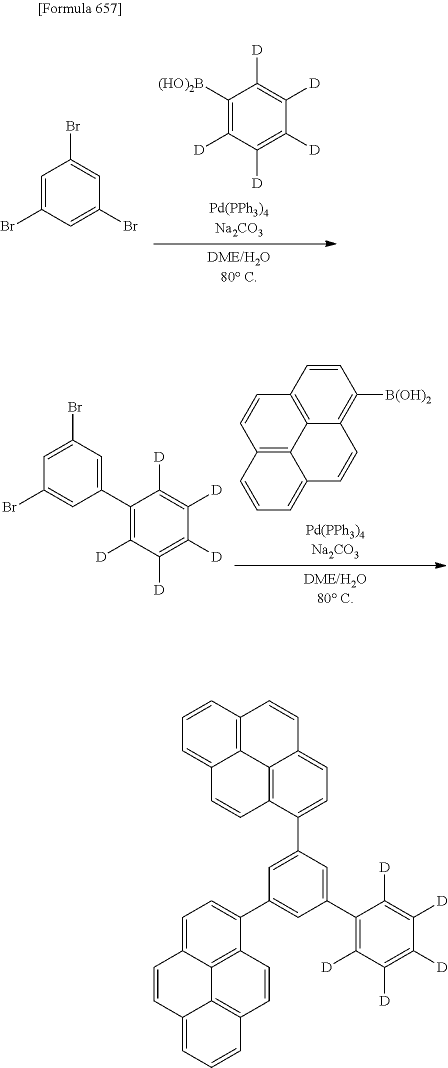

In Synthesis Example 11, the compound BH1-86 was synthesized using 1,3,5-tribromobenzene in place of 1,3,5-tribromobenzene-2,4,6-d3 and using pyren-1-ylboronic acid in place of (pyren-1-yl-d9)boronic acid. Except for the above, the same reaction as in Synthesis Example 10 was performed to obtain a white solid. As a result of mass spectroscopy analysis, the white solid was the compound BH1-86, and m/e was equal to 560 while a calculated molecular weight was 559.72.

### Synthesis Example 12: Synthesis of Compound BH1-83

A compound BH1-83 was synthesized in accordance with a synthesis scheme below.

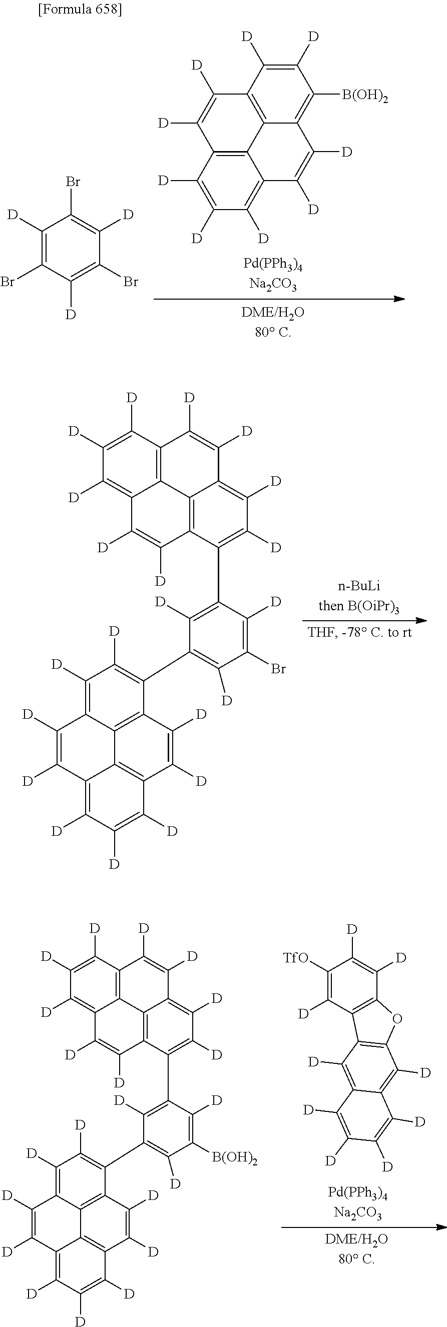

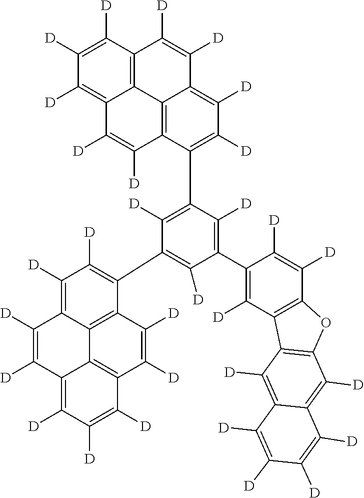 [SMILES: [2H]c1c(-c2c([2H])c([2H])c3oc4c([2H])c5c([2H])c([2H])c([2H])c([2H])c5c([2H])c4c3c2[2H])c([2H])c(-c2c([2H])c([2H])c3c([2H])c([2H])c4c([2H])c([2H])c([2H])c5c([2H])c([2H])c2c3c45)c([2H])c1-c1c([2H])c([2H])c2c([2H])c([2H])c3c([2H])c([2H])c([2H])c4c([2H])c([2H])c1c2c34]

### (1) Synthesis of 1,1′-(5-bromo-1,3-phenylene-2,4,6-d3)bis(pyrene-2,3,4,5,6,7,8,9,10-d9)

Under argon atmosphere, 4.20 g of 1,3,5-tribromobenzene-2,4,6-d3, 6.57 g of (pyren-1-yl-d9)boronic acid, 611 mg of tetrakis(triphenylphosphine)palladium(0), 46 mL of 1,2-dimethoxyethane, and 20 ml of 2M sodium carbonate aqueous solution were put into a flask and stirred at reflux for eight hours. The reaction solution was cooled to room temperature, and then extracted with toluene. After an aqueous phase was removed, an organic phase was washed with saturated saline. The organic phase was dried with anhydrous sodium sulfate and then concentrated. The residue was purified by silica gel column chromatography. The obtained sample was dried in a vacuum at room temperature for three hours to obtain 2.91 g of 1,1′-(5-bromo-1,3-phenylene-2,4,6-d3)bis(pyrene-2,3,4,5,6,7,8,9,10-d9) (a yield of 38%).

### (2) Synthesis of (3,5-bis(pyren-1-yl-d9)phenyl-2,4,6-d3)boronic acid

Under argon atmosphere, 2.91 g of 1,1′-(5-bromo-1,3-phenylene-2,4,6-d3)bis(pyrene-2,3,4,5,6,7,8,9,10-d9) and 25 mL of (dehydrated) tetrahydrofuran were put into a flask, and cooled to −78 degrees C. 3.9 mL of n-BuLi (1.55 M in hexane) was added thereto, and stirred for 30 minutes. Next, 2.0 mL of (ⁱPrO)₃B was added, stirred at −78 degrees C. for five minutes, and then stirred at room temperature for one hour. After the reaction, 25 mL of 1 M HCl aq. was added, and stirred at room temperature for one hour. The reaction solution was extracted with toluene. After an aqueous phase was removed, an organic phase was washed with saturated saline. The organic phase was dried with anhydrous sodium sulfate and concentrated, and then washed with hexane to obtain 2.10 g of (3,5-bis(pyren-1-yl-d9)phenyl-2,4,6-d3)boronic acid (a yield of 77%).

### (3) Synthesis of 2-(3,5-bis(pyren-1-yl-d9)phenyl-2,4,6-d3)naphtho[2,3-b]benzofuran-1,3,4,6,7,8,9,10,11-d9

Under argon atmosphere, (3,5-bis(pyren-1-yl-d9)phenyl-2,4,6-d3)boronic acid 2.10, 2.91 g of naphtho[2,3-b]benzofuran-2-yl-d9 trifluoromethanesulfonate, 247 mg of tetrakis(triphenylphosphine)palladium(0), 20 mL of 1,2-dimethoxyethane, and 8.0 ml of 2M sodium carbonate aqueous solution were put into a flask and stirred at reflux for eight hours. The reaction solution was cooled to room temperature, and then extracted with toluene. After an aqueous phase was removed, an organic phase was washed with saturated saline. The organic phase was dried with anhydrous sodium sulfate and then concentrated. The residue was purified by silica gel column chromatography. The obtained sample was dried in a vacuum at room temperature for three hours to obtain 1.43 g of 2-(3,5-bis(pyren-1-yl-d9)phenyl-2,4,6-d3)naphtho[2,3-b]benzofuran-1,3,4,6,7,8,9,10,11-d9 (a yield of 37%). As a result of mass spectroscopy analysis, the solid was the compound BH1-83, and m/e was equal to 725 while a calculated molecular weight was 725.01.

### EXPLANATION OF CODES

1 . . . organic EL device, 2 . . . substrate, 3 . . . anode, 4 . . . cathode, 51 . . . first emitting layer, 52 . . . second emitting layer, 6 . . . hole injecting layer, 7 . . . hole transporting layer, 8 . . . electron transporting layer, 9 . . . electron injecting layer

1. An organic electroluminescence device comprising:
an anode;
a cathode;
a first emitting layer provided between the anode and the cathode and comprising a first compound; and
a second emitting layer provided between the anode and the cathode and comprising a second compound, wherein
at least one of the first emitting layer or the second emitting layer comprises a compound having at least one deuterium atom, and
at least one of the first emitting layer or the second emitting layer comprises a compound having a fused ring that includes four or more rings.

2. The organic electroluminescence device according to claim 1, wherein
at least one of the first emitting layer or the second emitting layer comprises a compound having at least one deuterium atom and having a fused ring that includes four or more rings.

3. The organic electroluminescence device according to claim 1, wherein
the first compound comprises at least one deuterium atom.

4. The organic electroluminescence device according to claim 1, wherein
the first compound comprises a fused ring including four or more rings.

5. The organic electroluminescence device according to claim 1, wherein
the second compound comprises at least one deuterium atom.

6. The organic electroluminescence device according to claim 1, wherein
the second compound comprises a fused ring including four or more rings.

7. The organic electroluminescence device according to claim 1, wherein
one of the first compound and the second compound substantially comprises no deuterium atom.

8. The organic electroluminescence device according to claim 1, wherein
the first compound comprises at least one skeleton selected from the group consisting of a pyrene skeleton, a benzanthracene skeleton, a xanthene skeleton, a chrysene skeleton, a fluoranthene skeleton, a triphenylene skeleton, a benzoxanthene skeleton, and a benzophenanthrene skeleton.

9. The organic electroluminescence device according to claim 1, wherein
the second compound comprises at least one skeleton selected from the group consisting of a pyrene skeleton, a benzanthracene skeleton, a xanthene skeleton, a chrysene skeleton, a fluoranthene skeleton, a triphenylene skeleton, a benzoxanthene skeleton, and a benzophenanthrene skeleton.

10. The organic electroluminescence device according to any claim 1, wherein
the compound having the fused ring that includes four or more rings comprises no anthracene skeleton.

11. The organic electroluminescence device according to claim 1, wherein
the compound having the fused ring that includes four or more rings is a compound represented by a formula (1) below and having at least one group represented by a formula (11) below, 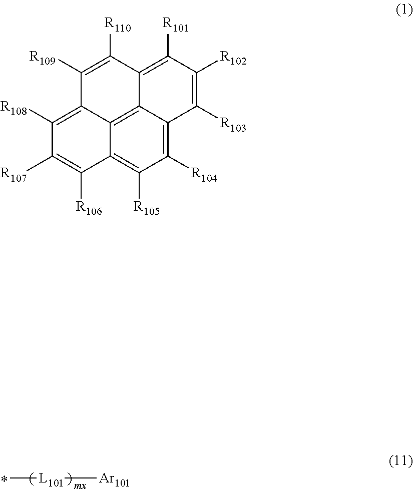
where, in the formula (1):
R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);
at least one of R₁₀₁ to R₁₁₀ is a group represented by the formula (11);
when a plurality of groups represented by the formula (11) are present, the plurality of groups represented by the formula (11) are mutually the same or different;
L₁₀₁ is a single bond, a substituted or unsubstituted arylene group having 6 to 50 ring carbon atoms, or a substituted or unsubstituted divalent heterocyclic group having 5 to 50 ring atoms;
Ar₁₀₁ is a substituted or unsubstituted aryl group having 6 to 50 ring carbon atoms, or a substituted or unsubstituted heterocyclic group having 5 to 50 ring atoms;
mx is 0, 1, 2, 3, 4, or 5;
when two or more L₁₀₁ are present, the two or more L₁₀₁ are mutually the same or different;
when two or more Ar₁₀₁ are present, the two or more Ar₁₀₁ are mutually the same or different;
* in the formula (11) represents a bonding position to a pyrene ring in the formula (1);
in the compound represented by the formula (1), R₉₀₁, R₉₀₂, R₉₀₃, R₉₀₄, R₉₀₅, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

12. The organic electroluminescence device according to claim 1, wherein
the compound having the fused ring that includes four or more rings is a compound represented by a formula (1X) below and having at least one group represented by a formula (11X) below, 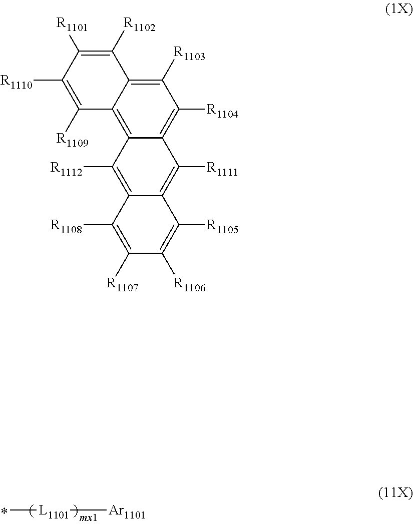
where, in the formula (1X):
R₁₁₀₁ to R₁₁₁₂ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11X);
at least one of R₁₁₀₁ to R₁₁₁₂ is a group represented by the formula (11X);
when a plurality of groups represented by the formula (11X) are present, the plurality of groups represented by the formula (11X) are mutually the same or different;
L₁₁₀₁ is a single bond, a substituted or unsubstituted arylene group having 6 to 50 ring carbon atoms, or a substituted or unsubstituted divalent heterocyclic group having 5 to 50 ring atoms;
Ar₁₁₀₁ is a substituted or unsubstituted aryl group having 6 to 50 ring carbon atoms, or a substituted or unsubstituted heterocyclic group having 5 to 50 ring atoms;
mx1 is 1, 2, 3, 4, or 5;
when two or more L₁₁₀₁ are present, the two or more L₁₁₀₁ are mutually the same or different;
when two or more Ar₁₁₀₁ are present, the two or more Ar₁₁₀₁ are mutually the same or different;
* in the formula (11X) represents a bonding position to a benz[a]anthracene ring in the formula (1X);
in the compound represented by the formula (1X), R₉₀₁, R₉₀₂, R₉₀₃, R₉₀₄, R₉₀₅, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

13. The organic electroluminescence device according to claim 1, wherein
the compound having the fused ring that includes four or more rings is a compound represented by a formula (14X) below and having at least one group represented by a formula (141) below, 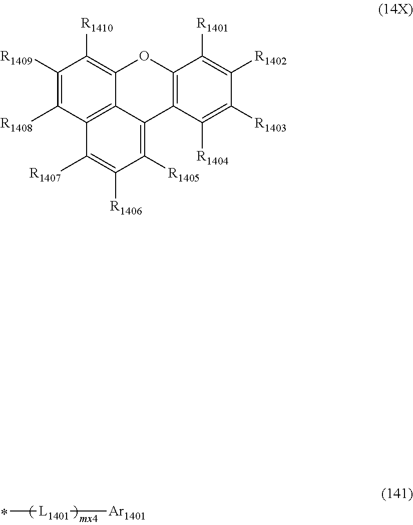
where, in the formula (14X):
R₁₄₀₁ to R₁₄₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (141);
at least one of R₁₄₀₁ to R₁₄₁₀ is a group represented by the formula (141);
when a plurality of groups represented by the formula (141) are present, the plurality of groups represented by the formula (141) are mutually the same or different;
L₁₄₀₁ is a single bond, a substituted or unsubstituted arylene group having 6 to 50 ring carbon atoms, or a substituted or unsubstituted divalent heterocyclic group having 5 to 50 ring atoms;
Ar₁₄₀₁ is a substituted or unsubstituted aryl group having 6 to 50 ring carbon atoms, or a substituted or unsubstituted heterocyclic group having 5 to 50 ring atoms;
mx4 is 0, 1, 2, 3, 4 or 5;
when two or more L₁₄₀₁ are present, the two or more L₁₄₀₁ are mutually the same or different;
when two or more Ar₁₄₀₁ are present, the two or more Ar₁₄₀₁ are mutually the same or different;
in the formula (141) represents a bonding position to a ring represented by the formula (14X);
in the compound represented by the formula (14X), R₉₀₁, R₉₀₂, R₉₀₃, R₉₀₄, R₉₀₅, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

14. The organic electroluminescence device according to claim 1, wherein
the second emitting layer comprises a compound represented by a formula (2) below, 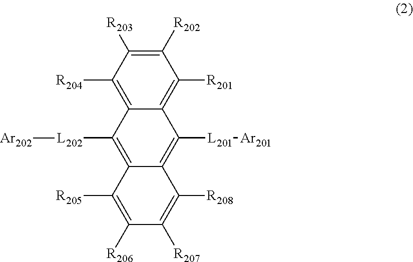
where, in the formula (2):
R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms;
Ar₂₀₁ and Ar₂₀₂ are each independently a substituted or unsubstituted aryl group having 6 to 50 ring carbon atoms, or a substituted or unsubstituted heterocyclic group having 5 to 50 ring atoms;
in the compound represented by the formula (2), R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

15. The organic electroluminescence device according to claim 14, wherein
at least one group of L₂₀₁, L₂₀₂, Ar₂₀₁, or Ar₂₀₂ comprises at least one deuterium atom.

16. The organic electroluminescence device according to claim 14, wherein
at least one of Ar₂₀₁ or Ar₂₀₂ is a group represented by a formula (21), a formula (22), a formula (23), or a formula (24) below, 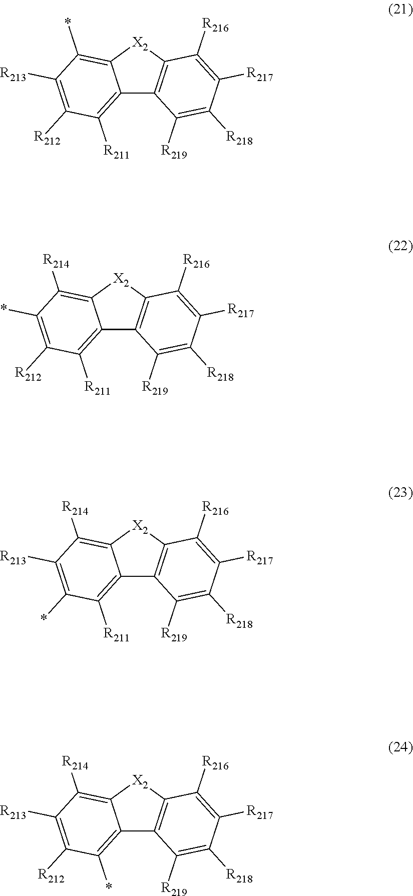
where, in the formulae (21) to (24):
X₂ is an oxygen atom, a sulfur atom, CR₂₃₁R₂₃₂, or NR₂₃₃;
at least one combination of adjacent two or more of R₂₁₁ to R₂₁₄ and R₂₁₆ to R₂₁₉ are mutually bonded to form a substituted or unsubstituted monocyclic ring, mutually bonded to form a substituted or unsubstituted fused ring, or not mutually bonded;
a combination of R₂₃₁ and R₂₃₂ are mutually bonded to form a substituted or unsubstituted monocyclic ring, mutually bonded to form a substituted or unsubstituted fused ring, or not mutually bonded;
R₂₁₁ to R₂₁₄ and R₂₁₆ to R₂₁₉ forming neither the substituted or unsubstituted monocyclic ring nor the substituted or unsubstituted fused ring; R₂₃₁ and R₂₃₂ forming neither the substituted or unsubstituted monocyclic ring nor the substituted or unsubstituted fused ring; and R₂₃₃ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
* in each of the formulae (21) to (24) represents a bonding position to L₂₀₁ or L₂₀₂.

17. The organic electroluminescence device according to claim 14, wherein
at least one of L₂₀₁ or L₂₀₂ is a group represented by a formula (L21), a formula (L22), a formula (L23), or a formula (L24) below, 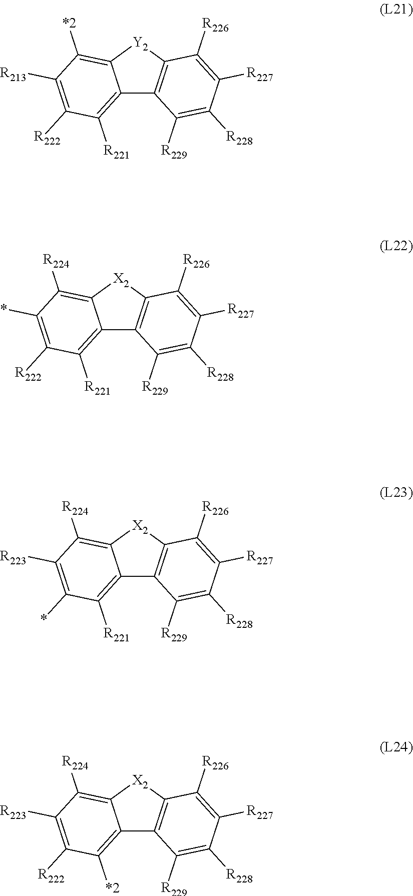
where, in the formulae (L21) to (L24):
Y₂ is an oxygen atom, a sulfur atom, CR₂₄₁R₂₄₂, or NR₂₄₃;
at least one of R₂₂₁ to R₂₂₄ or R₂₂₆ to R₂₂₉ is Ar₂₀₁ or Ar₂₀₂;
at least one combination of adjacent two or more of R₂₂₁ to R₂₂₄ and R₂₂₆ to R₂₂₉ being neither Ar₂₀₁ nor Ar₂₀₂ are mutually bonded to form a substituted or unsubstituted monocyclic ring, mutually bonded to form a substituted or unsubstituted fused ring, or not mutually bonded;
a combination of R₂₄₁ and R₂₄₂ are mutually bonded to form a substituted or unsubstituted monocyclic ring, mutually bonded to form a substituted or unsubstituted fused ring, or not mutually bonded;
R₂₂₁ to R₂₂₄ and R₂₂₆ to R₂₂₉ being neither Ar₂₀₁ nor Ar₂₀₂ and forming neither the substituted or unsubstituted monocyclic ring nor the substituted or unsubstituted fused ring; R₂₄₁ and R₂₄₂ forming neither the substituted or unsubstituted monocyclic ring nor the substituted or unsubstituted fused ring; and R₂₄₃ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
*2 in each of the formulae (L21) to (L24) represents a bonding position to an anthracene ring represented by the formula (2).

18. The organic electroluminescence device according to claim 11, wherein
the groups specified to be “substituted or unsubstituted” are each an “unsubstituted” group.

19. The organic electroluminescence device according to claim 1, wherein
the first emitting layer further comprises a third compound, and
the third compound is a compound that emits light having a maximum peak wavelength in a range from 430 nm to 480 nm.

20. The organic electroluminescence device according to claim 1, wherein
the first emitting layer comprises no metal complex.

21. The organic electroluminescence device according to claim 1, wherein
the second emitting layer further comprises a fourth compound, and
the fourth compound is a compound that emits light having a maximum peak wavelength in a range from 430 nm to 480 nm.

22. The organic electroluminescence device according to claim 1, wherein
the second emitting layer comprises no metal complex.

23. The organic electroluminescence device according to claim 1, wherein
the first compound is a host material.

24. The organic electroluminescence device according to claim 1, wherein
the second compound is a host material.

25. The organic electroluminescence device according to claim 1, wherein
a triplet energy T₁(M1) of the first compound is different from a triplet energy T₁(M2) of the second compound.

26. The organic electroluminescence device according to claim 1, wherein
the first emitting layer and the second emitting layer are in direct contact with each other.

27. The organic electroluminescence device according to claim 1, wherein
the second emitting layer is provided between the first emitting layer and the cathode.

28. The organic electroluminescence device according to claim 1, further comprising a hole transporting layer between the anode and one of the first emitting layer and the second emitting layer provided closer to the anode.

29. The organic electroluminescence device according to claim 1, further comprising an electron transporting layer between the cathode and one of the first emitting layer and the second emitting layer provided closer to the cathode.

30. An electronic device comprising the organic electroluminescence device according to claim 1.
